(19) United States
(12) Patent Application Publication
(10) Pub. No.: US 2024/0065102 A1
(43) Pub. Date: Feb. 22, 2024

(54) ORGANIC LIGHT EMITTING DEVICE

(71) Applicant: LG CHEM, LTD., Seoul (KR)

(72) Inventors: Minjun Kim, Daejeon (KR); Dong Hoon Lee, Daejeon (KR); Sang Duk Suh, Daejeon (KR); Young Seok Kim, Daejeon (KR)

(21) Appl. No.: 18/038,369

(22) PCT Filed: Jul. 22, 2022
(86) PCT No.: PCT/IB2022/056793

§ 371 Date: May 23, 2023

(30) Foreign Application Priority Data
Jul. 22, 2021 (KR) 10-2021-0096451
Jul. 22, 2022 (KR) 10-2022-0091091

(51) Int. Cl. H10K 85/60; C07D 405/04; C07D 405/14; C07D 409/14; C07D 405/10; C07F 7/08; H10K 85/40; C07D 209/86; C07D 405/12; C07D 209/88; C07D 409/12; C07D 209/80

(52) U.S. Cl. CPC H10K 85/6574; C07D 405/04; H10K 85/654; H10K 85/626; C07D 405/14; C07D 409/14; H10K 85/6576; H10K 85/622; C07D 405/10; H10K 85/615; C07F 7/0812; H10K 85/40; C07D 209/86; H10K 85/636; C07D 405/12; H10K 85/633; C07D 209/88; C07D 409/12; C07D 209/80; H10K 50/12

(57) ABSTRACT

An organic light emitting device comprising an anode, a cathode, and a light emitting layer between the anode and the cathode, the light emitting layer including a compound represented by Chemical Formula 1 and a compound represented by Chemical Formula 2, and having improved driving voltage, efficiency and lifetime is provided.

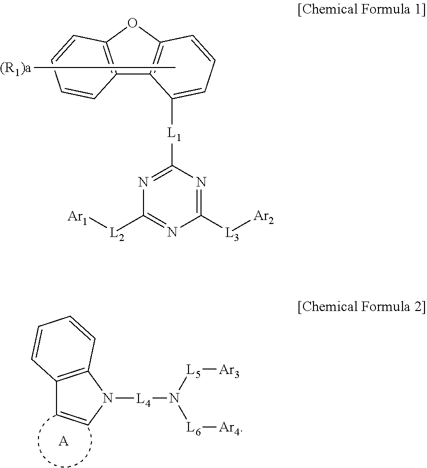

### CROSS-REFERENCE TO RELATED APPLICATION(S)

This application is a National Phase entry pursuant to 35 U.S.C. § 371 of International Application No. PCT/IB2022/056793 filed on Jul. 22, 2022, and claims priority to and the benefit of Korean Patent Application No. 10-2021-0096451 filed on Jul. 22, 2021 and Korean Patent Application No. 10-2022-0091091 filed on Jul. 22, 2022, the disclosures of which are incorporated herein by reference in their entirety.

### FIELD OF DISCLOSURE

The present disclosure relates to an organic light emitting device having improved driving voltage, efficiency and lifetime.

### BACKGROUND

In general, an organic light emitting phenomenon refers to a phenomenon where electric energy is converted into light energy by using an organic material. The organic light emitting device using the organic light emitting phenomenon has characteristics such as a wide viewing angle, an excellent contrast, a fast response time, an excellent luminance, driving voltage and response speed, and thus many studies have proceeded.

The organic light emitting device generally has a structure which comprises an anode, a cathode, and an organic material layer between the anode and the cathode. The organic material layer frequently has a multilayered structure that comprises different materials in order to enhance efficiency and stability of the organic light emitting device, and for example, the organic material layer may be formed of a hole injection layer, a hole transport layer, a light emitting layer, an electron transport layer, an electron injection layer and the like. In the structure of the organic light emitting device, if a voltage is applied between two electrodes, the holes are injected from an anode into the organic material layer and the electrons are injected from the cathode into the organic material layer, and when the injected holes and electrons meet each other, an exciton is formed, and light is emitted when the exciton falls to a ground state again.

There is a continuing need for the development of an organic material used in the organic light emitting device as described above.

### RELATED ART

Korean Unexamined Patent Publication No. 10-2000-0051826

### SUMMARY

It is an object of the present disclosure to provide an organic light emitting device having improved driving voltage, efficiency and lifetime.

According to the present disclosure, there is provided the following organic light emitting device:
an organic light emitting device comprising:
an anode;
a cathode; and
a light emitting layer between the anode and the cathode,
wherein the light emitting layer includes a compound represented by the following Chemical Formula 1 and a compound represented by the following Chemical Formula 2:

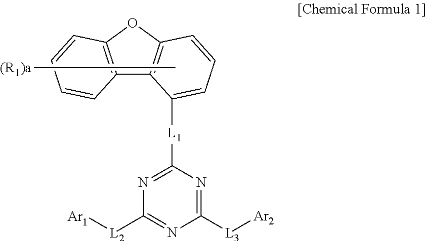

in Chemical Formula 1,
Ar₁ and Ar₂ are each independently a substituted or unsubstituted C₆₋₆₀ aryl; or a substituted or unsubstituted C₂₋₆₀ heteroaryl containing one or more selected from the group consisting of N, O and S,
L₁ is a single bond or one selected from the group consisting of

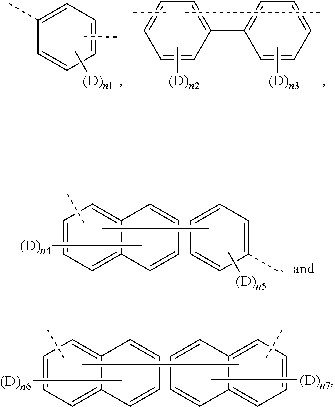

wherein D is deuterium, n1 to n3 and n5 are each independently an integer of 0 to 4, and n4, n6 and n7 are each independently an integer of 0 to 6,
L₂ and L₃ are each independently a single bond; or a substituted or unsubstituted C₆₋₆₀ arylene,
R₁ is each independently hydrogen; deuterium; a substituted or unsubstituted C₆₋₆₀ aryl; or a substituted or unsubstituted C₂₋₆₀ heteroaryl containing one or more selected from the group consisting of N, O and S, with the proviso that at least one R₁ is deuterium, phenyl substituted with deuterium, a substituted or unsubstituted C₁₀₋₆₀ aryl or a substituted or unsubstituted C₂₋₆₀ heteroaryl containing one or more selected from the group consisting of N, O and S, and
a is an integer of 1 to 7,
wherein the compound represented by Chemical Formula 1 may not contain any deuterium or may contain at least one deuterium,

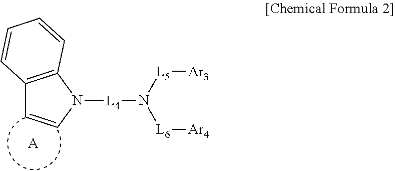

in Chemical Formula 2,
A is a benzene ring or a naphthalene ring,
Ar₃ and Ar₄ are each independently a substituted or unsubstituted C₆₋₆₀ aryl; or a substituted or unsubstituted C₂₋₆₀ heteroaryl containing one or more selected from the group consisting of N, O and S,
L₄ is a substituted or unsubstituted C₆₋₆₀ arylene, and
L₅ and L₆ are each independently a single bond, a substituted or unsubstituted C₆₋₆₀ arylene, or a substituted or unsubstituted C₂₋₆₀ heteroarylene containing one or more selected from the group consisting of N, O and S.

The above-mentioned organic light emitting device includes the compound represented by Chemical Formula 1 and the compound represented by Chemical Formula 2 in the light emitting layer, and thus can improve the efficiency, achieve low driving voltage and/or improve lifetime characteristics in the organic light emitting device.

### BRIEF DESCRIPTION OF THE DRAWINGS

FIG. 1 shows an example of an organic light emitting device comprising a substrate 1, an anode 2, a light emitting layer 3, and a cathode 4.

FIG. 2 shows an example of an organic light emitting device comprising a substrate 1, an anode 2, a hole injection layer 5, a hole transport layer 6, an electron blocking layer 7, a light emitting layer 3, a hole blocking layer 8, an electron injection and transport layer 9, and a cathode 4.

### DETAILED DESCRIPTION

Hereinafter, embodiments of the present disclosure will be described in more detail to facilitate understanding of the invention.

As used herein, the notation

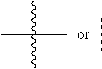

means a bond linked to another substituent group.

As used herein, the term “substituted or unsubstituted” means being unsubstituted or substituted with one or more substituents selected from the group consisting of deuterium; a halogen group; a nitrile group; a nitro group; a hydroxy group; a carbonyl group; an ester group; an imide group; an amino group; a phosphine oxide group; an alkoxy group; an aryloxy group; an alkylthioxy group; an arylthioxy group; an alkylsulfoxy group; an arylsulfoxy group; a silyl group; a boron group; an alkyl group; a cycloalkyl group; an alkenyl group; an aryl group; an aralkyl group; an aralkenyl group; an alkylaryl group; an alkylamine group; an aralkylamine group; a heteroarylamine group; an arylamine group; an arylphosphine group; and a heteroaryl group containing at least one of N, O and S atoms, or being unsubstituted or substituted with a substituent from the above substituent group which is further substituted by one or more selected from the above substituent group.

In the present disclosure, the carbon number of a carbonyl group is not particularly limited, but is preferably 1 to 40. Specifically, the carbonyl group may be a substituent having the following structural formulas, but is not limited thereto.

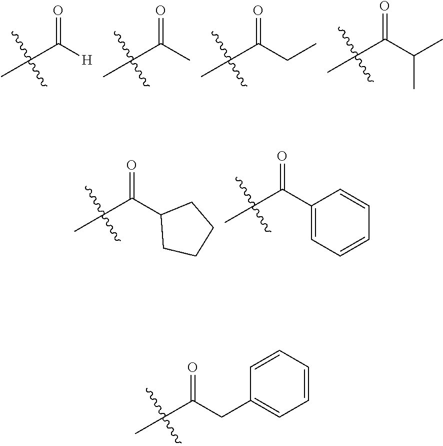

In the present disclosure, an ester group may have a structure in which oxygen of the ester group may be substituted by a straight-chain, branched-chain, or cyclic alkyl group having 1 to 25 carbon atoms, or an aryl group having 6 to 25 carbon atoms. Specifically, the ester group may be a substituent having the following structural formulas, but is not limited thereto.

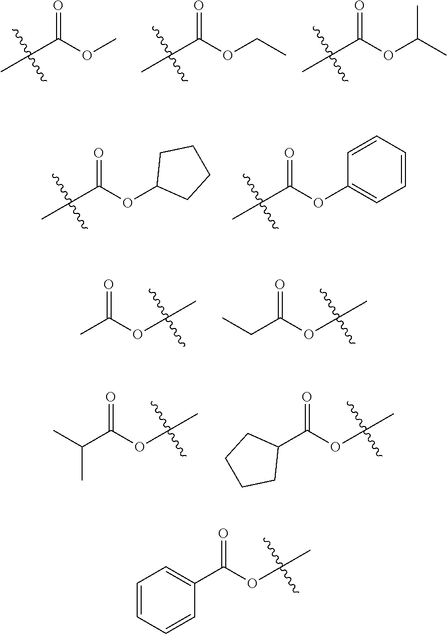

In the present disclosure, the carbon number of an imide group is not particularly limited, but is preferably 1 to 25. Specifically, the imide group may be a substituent having the following structural formulas, but is not limited thereto.

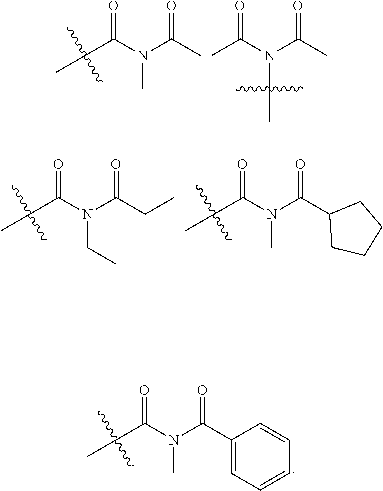

In the present disclosure, a silyl group specifically includes a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group and the like, but is not limited thereto.

In the present disclosure, a boron group specifically includes a trimethylboron group, a triethylboron group, a t-butyldimethylboron group, a triphenylboron group, and a phenylboron group, but is not limited thereto.

In the present disclosure, examples of a halogen group include fluorine, chlorine, bromine, or iodine.

In the present disclosure, the alkyl group may be straight-chain or branched-chain, and the carbon number thereof is not particularly limited, but is preferably 1 to 40. According to one embodiment, the carbon number of the alkyl group is 1 to 20. According to another embodiment, the carbon number of the alkyl group is 1 to 10. According to another embodiment, the carbon number of the alkyl group is 1 to 6. Specific examples of the alkyl group include methyl, ethyl, propyl, n-propyl, isopropyl, butyl, n-butyl, isobutyl, tert-butyl, sec-butyl, 1-methyl-butyl, 1-ethyl-butyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cyclohexylmethyl, octyl, n-octyl, tert-octyl, 1-methylheptyl, 2-ethylhexyl, 2-propylpentyl, n-nonyl, 2,2-dimethylheptyl, 1-ethyl-propyl, 1,1-dimethyl-propyl, isohexyl, 2-methylpentyl, 4-methylhexyl, 5-methylhexyl, and the like, but are not limited thereto.

In the present disclosure, the alkenyl group may be straight-chain or branched-chain, and the carbon number thereof is not particularly limited, but is preferably 2 to 40. According to one embodiment, the carbon number of the alkenyl group is 2 to 20. According to another embodiment, the carbon number of the alkenyl group is 2 to 10. According to still another embodiment, the carbon number of the alkenyl group is 2 to 6. Specific examples thereof include vinyl, 1-propenyl, isopropenyl, 1-butenyl, 2-butenyl, 3-butenyl, 1-pentenyl, 2-pentenyl, 3-pentenyl, 3-methyl-1-butenyl, 1,3-butadienyl, allyl, 1-phenylvinyl-1-yl, 2-phenylvinyl-1-yl, 2,2-diphenylvinyl-1-yl, 2-phenyl-2-(naphthyl-1-yl)vinyl-1-yl, 2,2-bis(diphenyl-1-yl)vinyl-1-yl, a stilbenyl group, a styrenyl group, and the like, but are not limited thereto.

In the present disclosure, a cycloalkyl group is not particularly limited, but the carbon number thereof is preferably 3 to 60. According to one embodiment, the carbon number of the cycloalkyl group is 3 to 30. According to another embodiment, the carbon number of the cycloalkyl group is 3 to 20. According to still another embodiment, the carbon number of the cycloalkyl group is 3 to 6. Specific examples thereof include cyclopropyl, cyclobutyl, cyclopentyl, 3-methylcyclopentyl, 2,3-dimethylcyclopentyl, cyclohexyl, 3-methylcyclohexyl, 4-methylcyclohexyl, 2,3-dimethylcyclohexyl, 3,4,5-trimethylcyclohexyl, 4-tert-butylcyclohexyl, cycloheptyl, cyclooctyl, and the like, but are not limited thereto.

In the present disclosure, an aryl group is not particularly limited, but the carbon number thereof is preferably 6 to 60, and it may be a monocyclic aryl group or a polycyclic aryl group. According to one embodiment, the carbon number of the aryl group is 6 to 30. According to one embodiment, the carbon number of the aryl group is 6 to 20. The aryl group may be a phenyl group, a biphenyl group, a terphenyl group or the like as the monocyclic aryl group, but is not limited thereto. The polycyclic aryl group includes a naphthyl group, an anthracenyl group, a phenanthrenyl group, a pyrenyl group, a perylenyl group, a chrysenyl group, or the like, but is not limited thereto.

In the present disclosure, the fluorenyl group may be substituted, and two substituents may be linked with each other to form a spiro structure. In the case where the fluorenyl group is substituted,

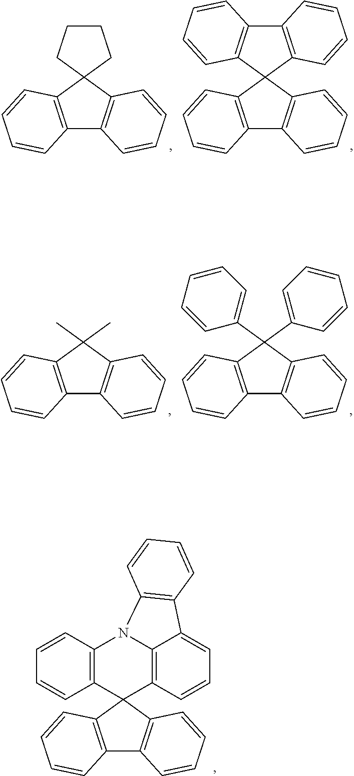

and the like can be formed. However, the structure is not limited thereto.

In the present disclosure, a heteroaryl group is a heteroaryl group containing at least one of O, N, Si and S as a heteroatom, and the carbon number thereof is not particularly limited, but is preferably 2 to 60. According to an exemplary embodiment of heteroaryl, the heteroaryl group has 6 to 30 carbon atoms. According to an exemplary embodiment, the heteroaryl group has 6 to 20 carbon atoms. Examples of heteroaryl groups include a thiophene group, a furan group, a pyrrole group, an imidazole group, a thiazole group, an oxazol group, an oxadiazol group, a triazol group, a pyridyl group, a bipyridyl group, a pyrimidyl group, a triazine group, an acridyl group, a pyridazine group, a pyrazinyl group, a quinolinyl group, a quinazoline group, a quinoxalinyl group, a phthalazinyl group, a pyridopyrimidinyl group, a pyridopyrazinyl group, a pyrazinopyrazinyl group, an isoquinoline group, an indole group, a carbazole group, a benzoxazole group, a benzoimidazole group, a benzothiazol group, a benzocarbazole group, a benzothiophene group, a dibenzothiophene group, a benzofuranyl group, a phenanthroline group, an isoxazolyl group, a thiadiazolyl group, a phenothiazinyl group, a dibenzofuranyl group, and the like, but are not limited thereto.

In the present disclosure, the aryl group in the aralkyl group, the aralkenyl group, the alkylaryl group and the arylamine group is the same as the above-mentioned examples of the aryl group. In the present disclosure, the alkyl group in the aralkyl group, the alkylaryl group and the alkylamine group is the same as the above-mentioned examples of the alkyl group. In the present disclosure, the heteroaryl in the heteroarylamine may be applied to the above-mentioned description of the heteroaryl group. In the present disclosure, the alkenyl group in the aralkenyl group is the same as the above-mentioned examples of the alkenyl group. In the present disclosure, the above-mentioned description of the aryl group may be applied except that the arylene is a divalent group. In the present disclosure, the above-mentioned description of the heteroaryl group may be applied except that the heteroarylene is a divalent group. In the present disclosure, the above-mentioned description of the aryl group or cycloalkyl group may be applied except that the hydrocarbon ring is not a monovalent group but formed by combining two substituent groups. In the present disclosure, the above-mentioned description of the heteroaryl group may be applied, except that the heteroaryl is not a monovalent group but formed by combining two substituent groups.

In the present disclosure, the term “deuterated or substituted with deuterium” means that at least one available hydrogen in each Chemical Formula is substituted with deuterium. Specifically, “substituted with deuterium” in the definition of each Chemical Formula or substituent means that at least one or more positions at which hydrogen can be bonded in the molecule are substituted with deuterium.

Additionally, in the present disclosure, the term “deuterium substitution rate” means the percentage of the number of substituted deuterium relative to the total number of hydrogens that may be present in each chemical formula.

Below, the present disclosure will be described in detail for each configuration.

Anode and Cathode

An anode and a cathode used in the present disclosure mean electrodes used in an organic light emitting device.

As the anode material, generally, a material having a large work function is preferably used so that holes can be smoothly injected into the organic material layer. Specific examples of the anode material include metals such as vanadium, chrome, copper, zinc, and gold, or an alloy thereof; metal oxides such as zinc oxides, indium oxides, indium tin oxides (ITO), and indium zinc oxides (IZO); a combination of metals and oxides, such as ZnO:Al or SNO₂:Sb; conductive polymers such as poly(3-methylthiophene), poly[3,4-(ethylene-1,2-dioxy)thiophene](PEDOT), polypyrrole, and polyaniline, and the like, but are not limited thereto.

As the cathode material, generally, a material having a small work function is preferably used so that electrons can be easily injected into the organic material layer. Specific examples of the cathode material include metals such as magnesium, calcium, sodium, potassium, titanium, indium, yttrium, lithium, gadolinium, aluminum, silver, tin, and lead, or an alloy thereof; a multilayered structure material such as LiF/Al or LiO₂/Al, and the like, but are not limited thereto.

Hole Injection Layer

The organic light emitting device according to the present disclosure may further include a hole injection layer on the anode, if necessary.

The hole injection layer is a layer for injecting holes from the electrode, and the hole injection material is preferably a compound which has a capability of transporting the holes, thus has a hole injecting effect in the anode and an excellent hole injecting effect to the light emitting layer or the light emitting material, prevents excitons produced in the light emitting layer from moving to a hole injection layer or the electron injection material, and further is excellent in the ability to form a thin film. Further, it is preferable that a HOMO (highest occupied molecular orbital) of the hole injection material is between the work function of the anode material and a HOMO of a peripheral organic material layer.

Specific examples of the hole injection material include metal porphyrine, oligothiophene, an arylamine-based organic material, a hexanitrilehexaazatriphenylene-based organic material, a quinacridone-based organic material, a perylene-based organic material, anthraquinone, polyaniline and polythiophene-based conductive compound, and the like, but are not limited thereto.

Hole Transport Layer

The organic light emitting device according to the present disclosure may include a hole transport layer on the anode (or on the hole injection layer if the hole injection layer exists), if necessary.

The hole transport layer is a layer that can receive the holes from the anode or the hole injection layer and transport the holes to the light emitting layer, and the hole transport material is suitably a material having large mobility to the holes, which may receive holes from the anode or the hole injection layer and transfer the holes to the light emitting layer.

Specific examples thereof include an arylamine-based organic material, a conductive polymer, a block copolymer in which a conjugate portion and a non-conjugate portion are present together, and the like, but are not limited thereto.

Electron Blocking Layer

The organic light emitting device according to the present disclosure may include an electron blocking layer between a hole transport layer and a light emitting layer, if necessary. The electron blocking layer refers to a layer which is formed on the hole transport layer, and preferably, is provided in contact with the light emitting layer, and thus serves to control hole mobility, to prevent excessive movement of electrons, and to increase the probability of hole-electron bonding, thereby improving the efficiency of the organic light emitting device. The electron blocking layer includes an electron blocking material, and as an example of such an electron blocking material, an arylamine-based organic material or the like can be used, but is not limited thereto.

Light Emitting Layer

The light emitting layer used in the present disclosure is a layer that can emit light in the visible light region by combining holes and electrons transported from the anode and the cathode. Generally, the light emitting layer includes a host material and a dopant material, and in the present disclosure, the light emitting layer includes the compound represented by Chemical Formula 1 and the compound represented by Chemical Formula 2 as a host.

The compound represented by Chemical Formula 1 may contain no deuterium or may contain at least one deuterium.

Preferably, Ar₁ and Ar₂ may be each independently a substituted or unsubstituted C₆₋₂₀ aryl; or a substituted or unsubstituted C₂₋₂₀ heteroaryl containing one or more selected from the group consisting of N, O and S.

More preferably, Ar₁ and Ar₂ may be each independently phenyl, biphenylyl, terphenylyl, naphthyl, phenanthrenyl, dibenzofuranyl, dibenzothiophenyl, or triphenylsilyl phenyl, each of which may be unsubstituted or substituted with deuterium.

Most preferably, Ar₁ and Ar₂ may be each independently one selected from the following:

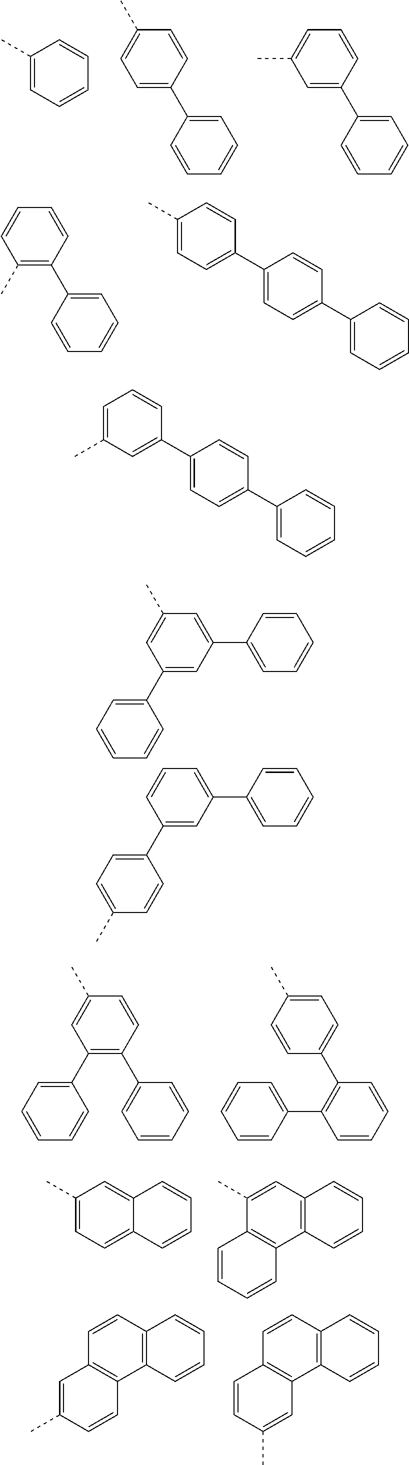

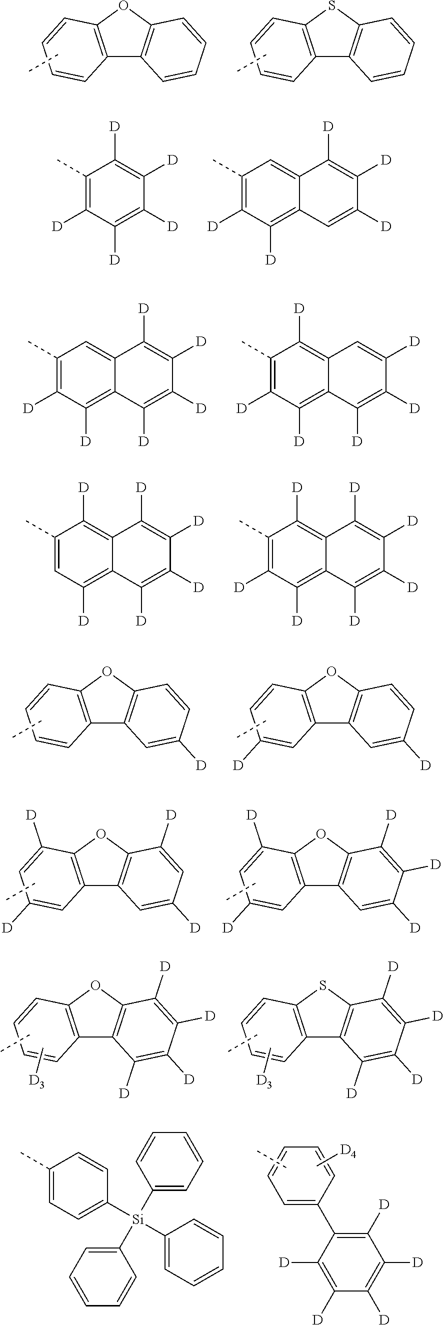

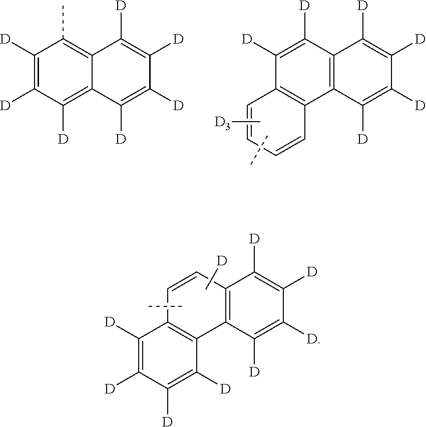

Preferably L₁ may be a single bond or one selected from the following:

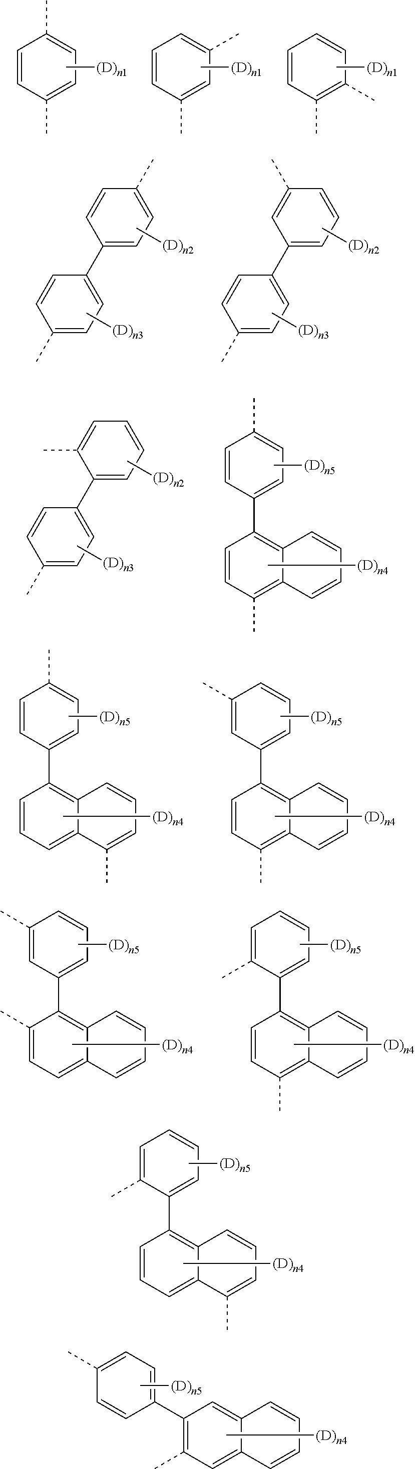

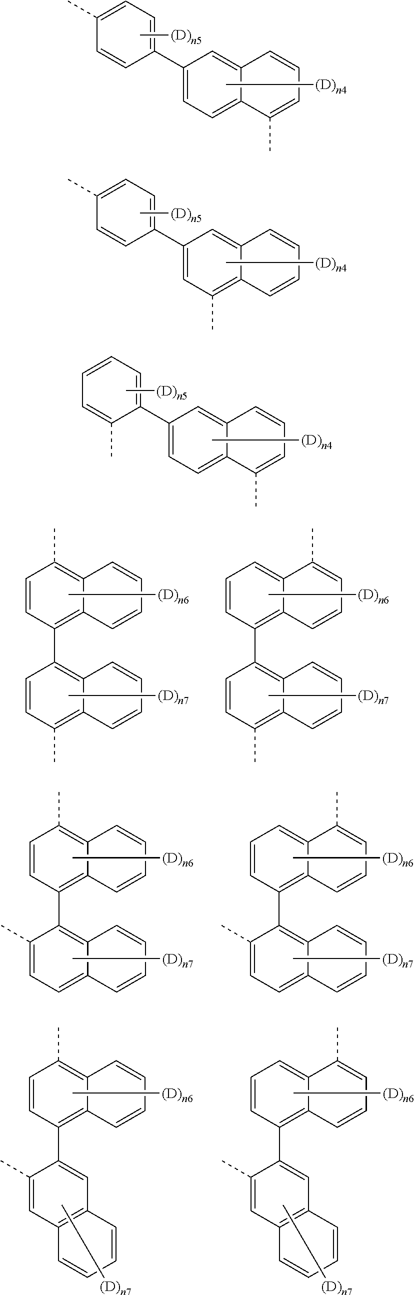

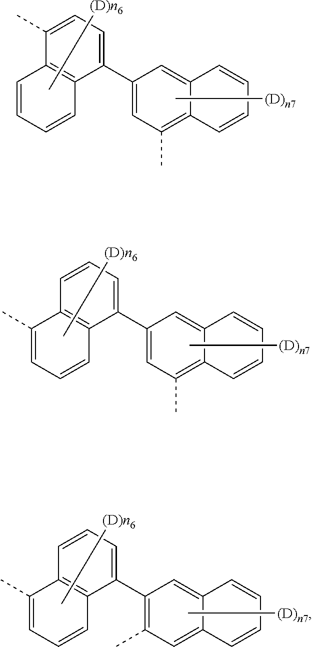

in the above listed groups, n1 to n7 are the same as defined in Chemical Formula 1.

Preferably, L₂ and L₃ may be each independently a single bond, or may be a substituted or unsubstituted C₆₋₂₀ arylene.

More preferably, L₂ and L₃ may be each independently a single bond, phenylene, biphenylylene, or naphthalenediyl, each of which, except a single bond, may be unsubstituted or substituted with deuterium.

In the present disclosure, a represents the number of R₁, and when a is two or more, two or more R₁ may be the same as or different from each other.

Preferably, R₁ is each independently hydrogen; deuterium; a substituted or unsubstituted C₆₋₂₀ aryl; or a substituted or unsubstituted C₂₋₂₀ heteroaryl containing one or more selected from the group consisting of N, O and S, with the proviso that at least one R₁ may be deuterium, phenyl substituted with deuterium, a substituted or unsubstituted C₁₀₋₂₀ aryl or a substituted or unsubstituted C₂₋₂₀ heteroaryl containing one or more selected from the group consisting of N, O and S.

More preferably, R₁ is each independently hydrogen, deuterium, phenyl, biphenylyl, terphenylyl, naphthyl, phenanthrenyl, triphenylenyl, fluoranthenyl, naphthyl phenyl, phenyl naphthyl, dihydroindenyl, dibenzofuranyl, dibenzothiophenyl, benzonaphthofuranyl, or benzonaphthothiophenyl, with the proviso that at least one R₁ may be deuterium, phenyl substituted with deuterium, biphenylyl, terphenylyl, naphthyl, phenanthrenyl, triphenylenyl, fluoranthenyl, naphthyl phenyl, phenyl naphthyl, dibenzofuranyl, dibenzothiophenyl, benzonaphthofuranyl, or benzonaphthothiophenyl, each of which, except for deuterium and phenyl substituted with deuterium, may be unsubstituted or substituted with deuterium.

Most preferably, R₁ may be each independently deuterium, phenyl substituted with deuterium, biphenylyl, terphenylyl, naphthyl, phenanthrenyl, triphenylenyl, fluoranthenyl, naphthyl phenyl, phenyl naphthyl, dihydroindenyl, dibenzofuranyl, dibenzothiophenyl, benzonaphthofuranyl, or benzonaphthothiophenyl, each of which, except for deuterium and phenyl substituted with deuterium, may be unsubstituted or substituted with deuterium.

Further, the compound represented by Chemical Formula 1 may contain no deuterium or may contain at least one deuterium.

As an example, when the compound contains deuterium, the deuterium substitution rate of the compound may be 1% to 100%. Specifically, the deuterium substitution rate of the compound may be 5% or more, 10% or more, 20% or more, 30% or more, 40% or more, 50% or more, 60% or more, 70% or more, 75% or more, 80% or more, or 90% or more, and less than 100%. The deuterium substitution rate of such a compound is calculated as the number of substituted deuterium relative to the total number of hydrogens that can be present in the Chemical Formula, wherein the number of substituted deuterium may be obtained through MALDI-TOF MS (Matrix-Assisted Laser Desorption/Ionization Time-of-Flight Mass Spectrometer) analysis.

Representative examples of the compound represented by Chemical Formula 1 are as follows:

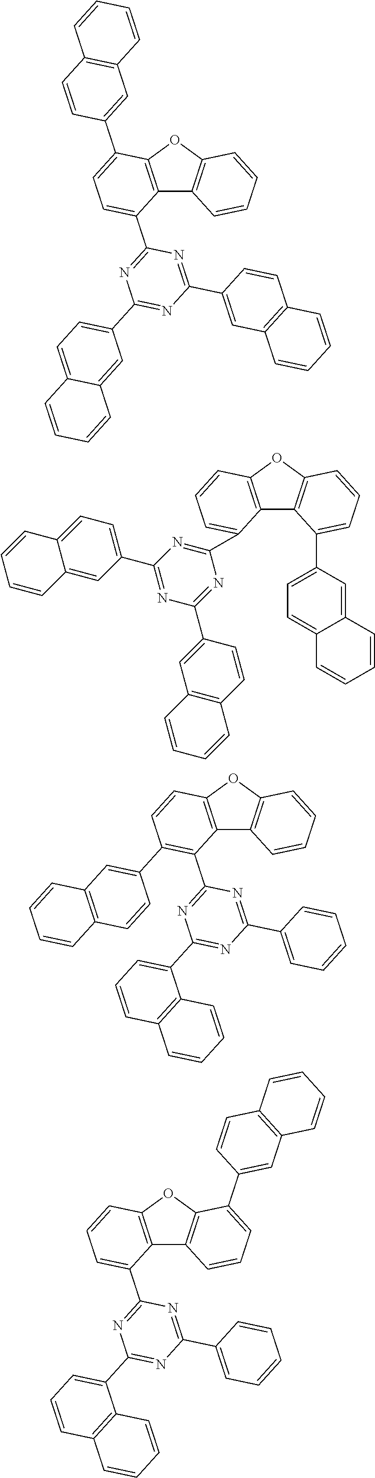

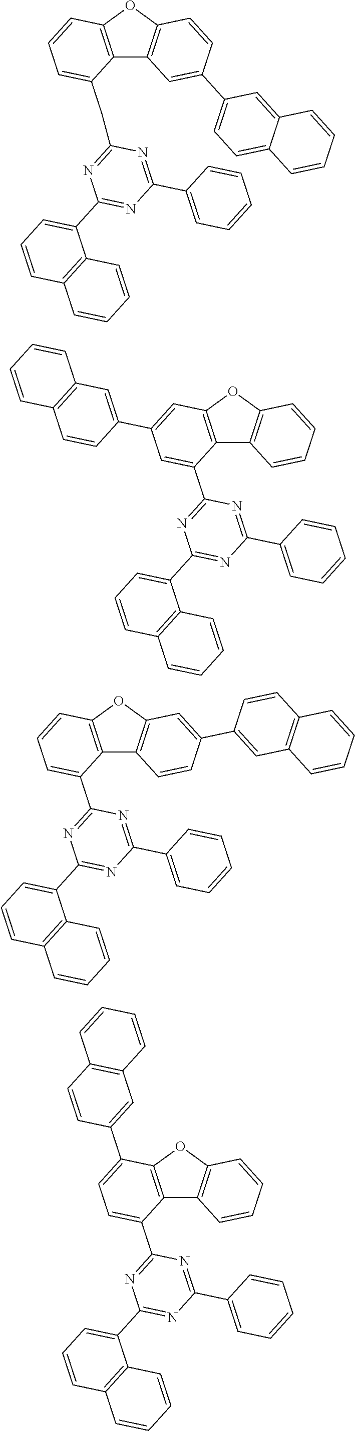

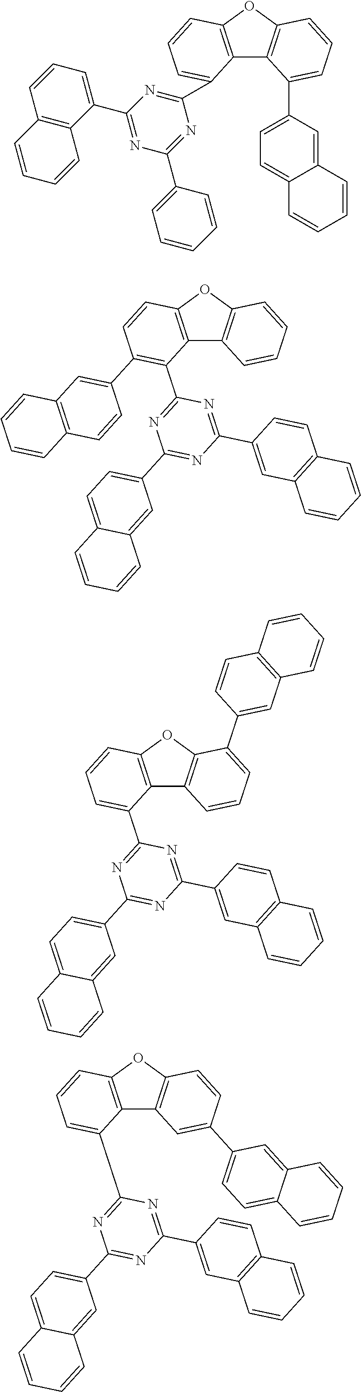

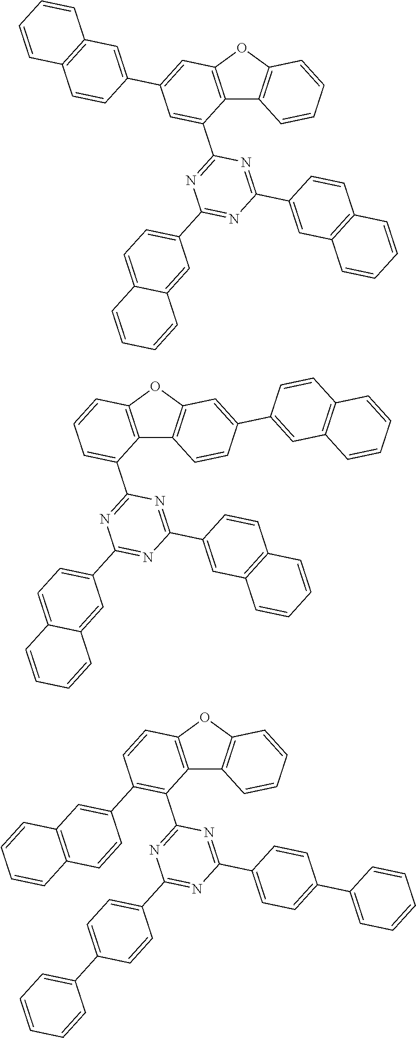

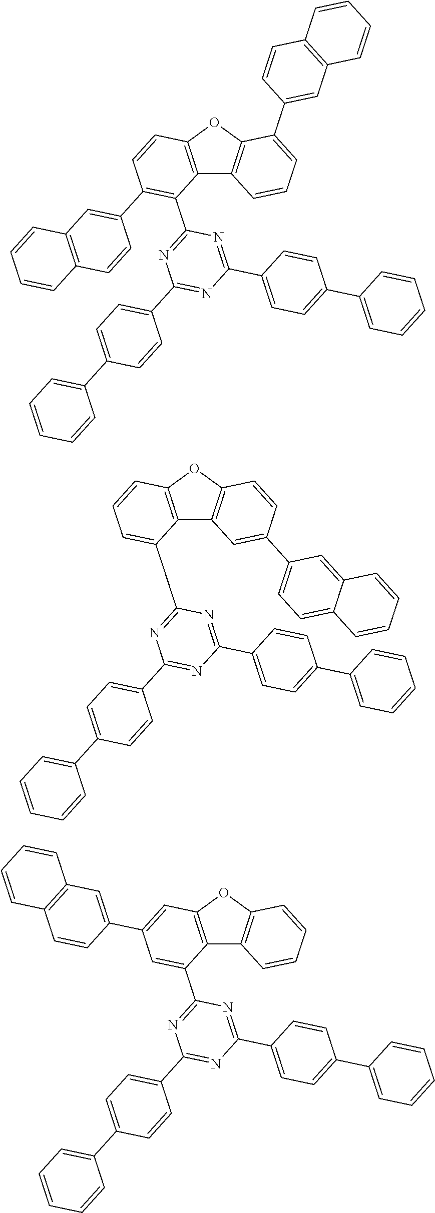

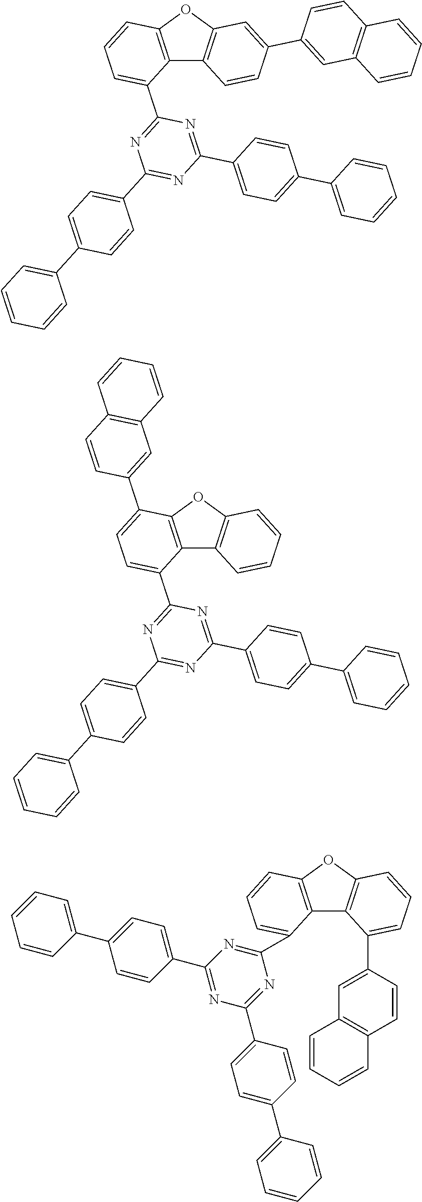 [SMILES: c1ccc(-c2ccc(-c3nc(-c4ccc(-c5ccccc5)cc4)nc(-c4cccc5oc6cc(-c7ccc8ccccc8c7)ccc6c45)n3)cc2)cc1 ; c1ccc(-c2ccc(-c3nc(-c4ccc(-c5ccccc5)cc4)nc(-c4ccc(-c5ccc6ccccc6c5)c5oc6ccccc6c45)n3)cc2)cc1 ; c1ccc(-c2ccc(-c3nc(-c4ccc(-c5ccccc5)cc4)nc(-c4cccc5oc6cccc(-c7ccc8ccccc8c7)c6c45)n3)cc2)cc1]

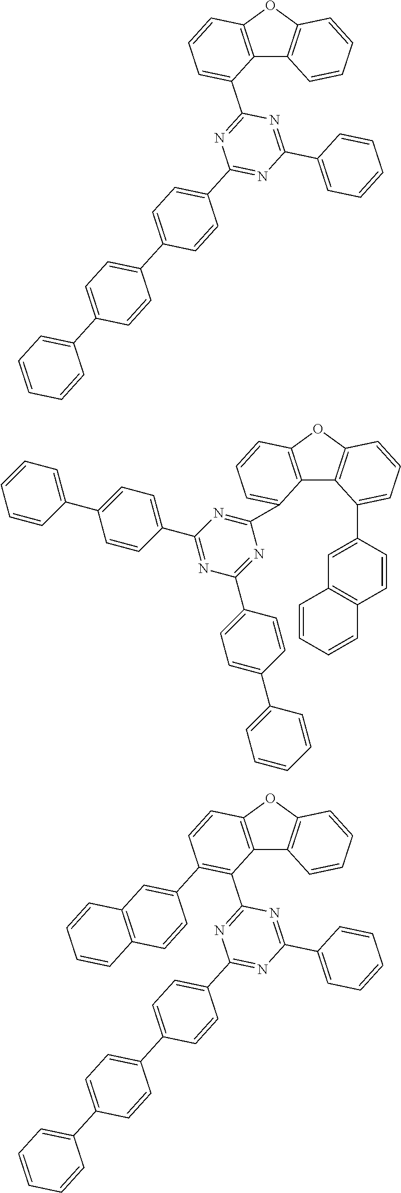

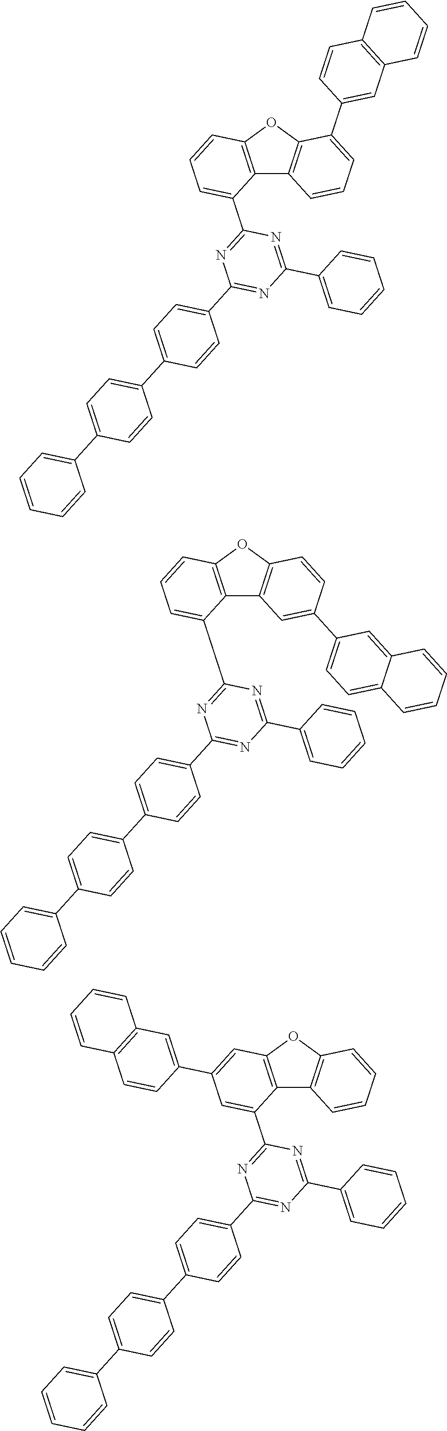 [SMILES: c1ccc(-c2ccc(-c3ccc(-c4nc(-c5ccccc5)nc(-c5cccc6oc7c(-c8ccc9ccccc9c8)cccc7c56)n4)cc3)cc2)cc1 ; c1ccc(-c2ccc(-c3ccc(-c4nc(Cc5cccc6oc7ccc(-c8ccc9ccccc9c8)cc7c56)nc(-c5ccccc5)n4)cc3)cc2)cc1 ; c1ccc(-c2ccc(-c3ccc(-c4nc(-c5ccccc5)nc(-c5cc(-c6ccc7ccccc7c6)cc6oc7ccccc7c56)n4)cc3)cc2)cc1]

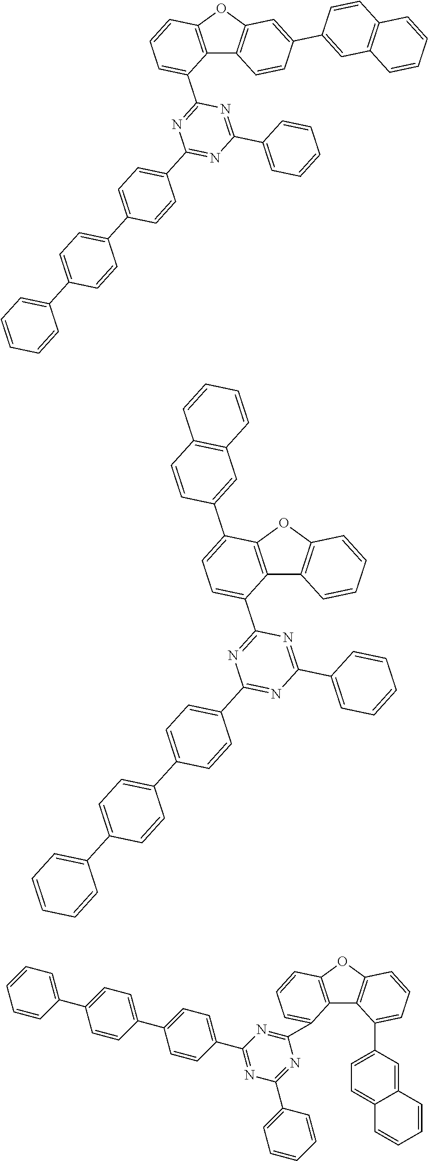 [SMILES: c1ccc(-c2ccc(-c3ccc(-c4nc(-c5ccccc5)nc(-c5cccc6oc7cc(-c8ccc9ccccc9c8)ccc7c56)n4)cc3)cc2)cc1 ; c1ccc(-c2ccc(-c3ccc(-c4nc(-c5ccccc5)nc(-c5ccc(-c6ccc7ccccc7c6)c6oc7ccccc7c56)n4)cc3)cc2)cc1 ; c1ccc(-c2ccc(-c3ccc(-c4nc(-c5ccccc5)nc(-c5cccc6oc7cccc(-c8ccc9ccccc9c8)c7c56)n4)cc3)cc2)cc1]

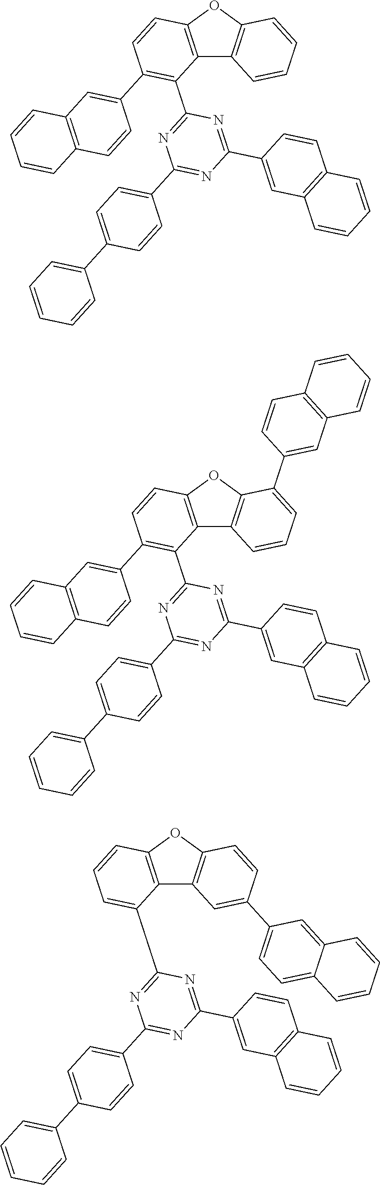 [SMILES: c1ccc(-c2ccc(-c3nc(-c4ccc5ccccc5c4)nc(-c4c(-c5ccc6ccccc6c5)ccc5oc6ccccc6c45)n3)cc2)cc1 ; c1ccc(-c2ccc(-c3nc(-c4ccc5ccccc5c4)nc(-c4c(-c5ccc6ccccc6c5)ccc5oc6c(-c7ccc8ccccc8c7)cccc6c45)n3)cc2)cc1 ; c1ccc(-c2ccc(-c3nc(Cc4cccc5oc6ccc(-c7ccc8ccccc8c7)cc6c45)nc(-c4ccc5ccccc5c4)n3)cc2)cc1]

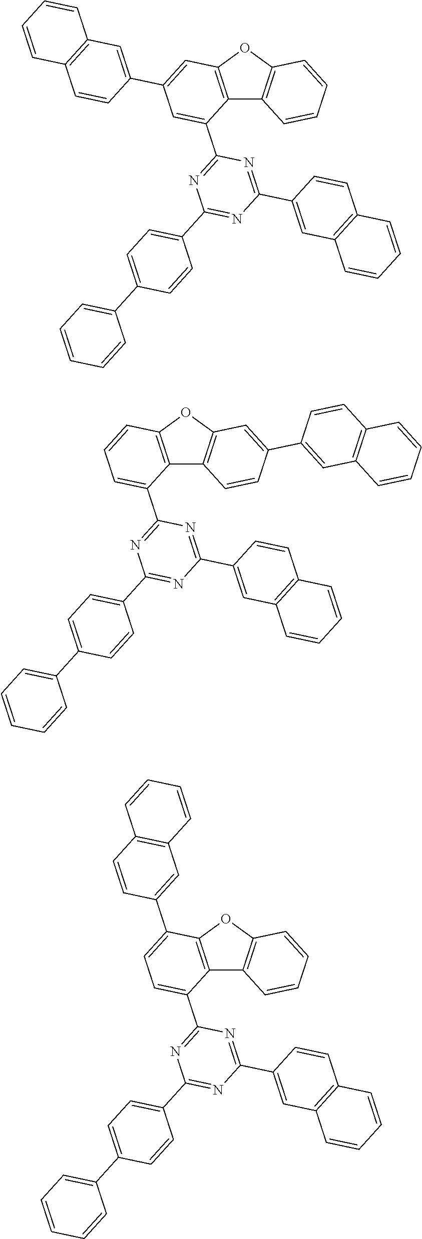 [SMILES: c1ccc(-c2ccc(-c3nc(-c4ccc5ccccc5c4)nc(-c4cc(-c5ccc6ccccc6c5)cc5oc6ccccc6c45)n3)cc2)cc1 ; c1ccc(-c2ccc(-c3nc(-c4ccc5ccccc5c4)nc(-c4cccc5oc6cc(-c7ccc8ccccc8c7)ccc6c45)n3)cc2)cc1 ; c1ccc(-c2ccc(-c3nc(-c4ccc5ccccc5c4)nc(-c4ccc(-c5ccc6ccccc6c5)c5oc6ccccc6c45)n3)cc2)cc1]

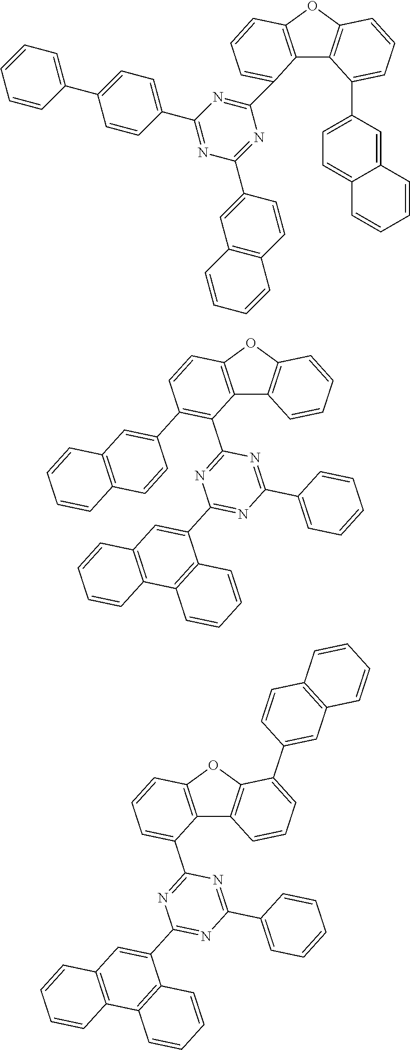

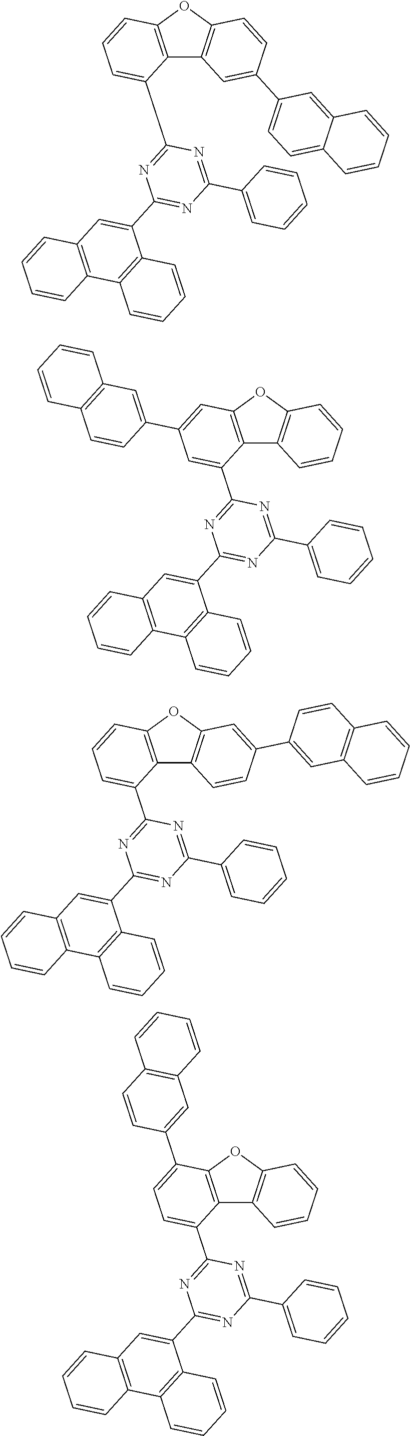

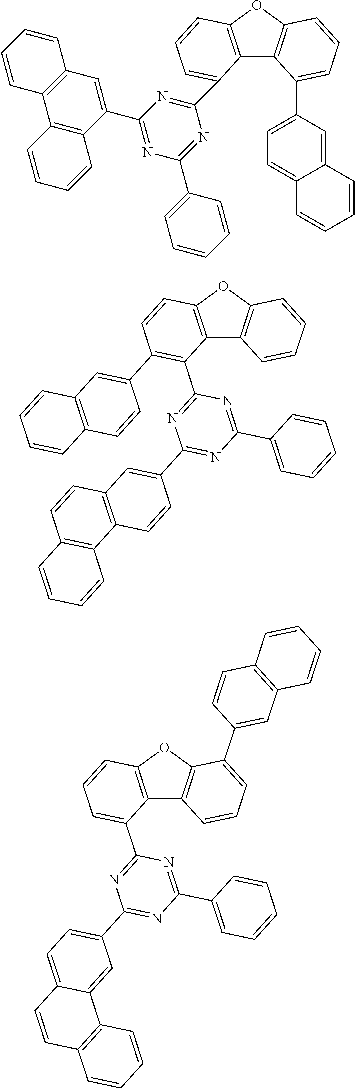

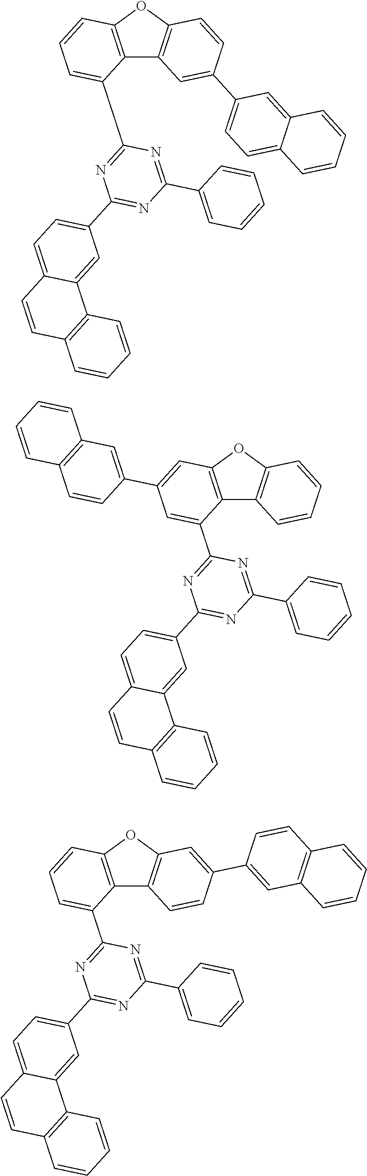

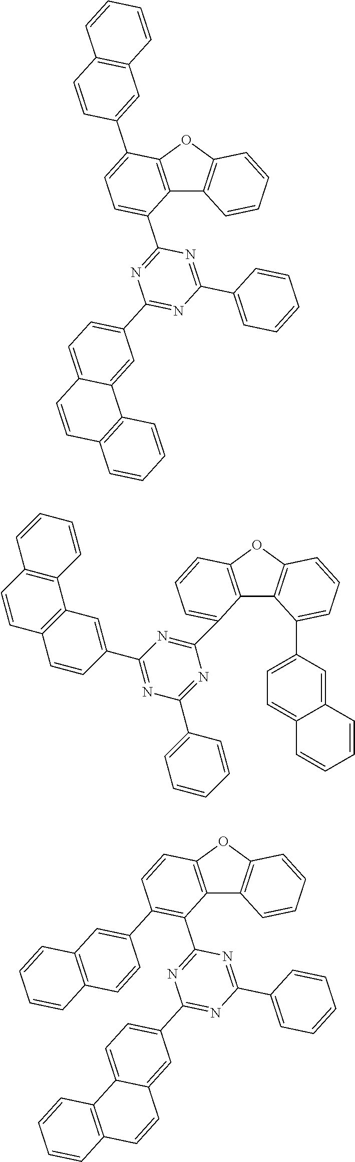 [SMILES: c1ccc(-c2nc(-c3ccc4ccc5ccccc5c4c3)nc(-c3ccc(-c4ccc5ccccc5c4)c4oc5ccccc5c34)n2)cc1 ; c1ccc(-c2nc(-c3ccc4ccc5ccccc5c4c3)nc(-c3cccc4oc5cccc(-c6ccc7ccccc7c6)c5c34)n2)cc1 ; c1ccc(-c2nc(-c3ccc4c(ccc5ccccc54)c3)nc(-c3c(-c4ccc5ccccc5c4)ccc4oc5ccccc5c34)n2)cc1]

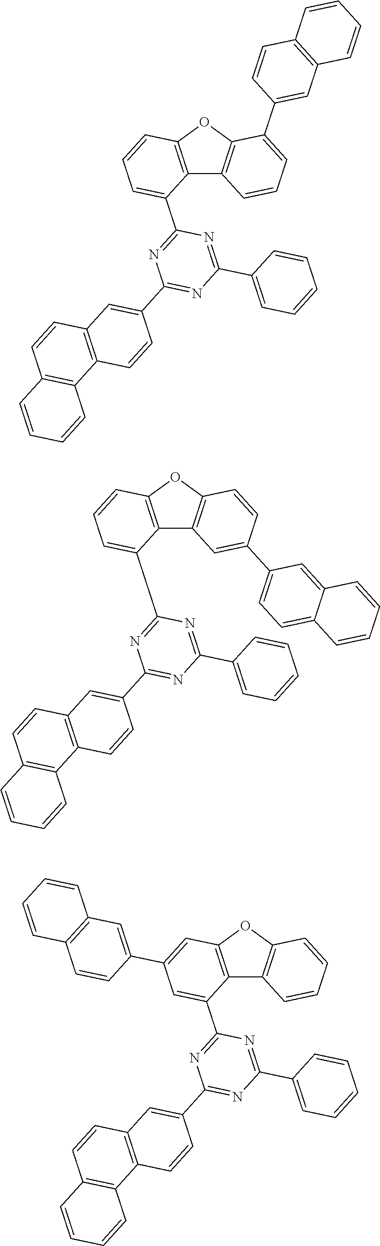 [SMILES: c1ccc(-c2nc(-c3ccc4c(ccc5ccccc54)c3)nc(-c3cccc4oc5c(-c6ccc7ccccc7c6)cccc5c34)n2)cc1 ; c1ccc(-c2nc(Cc3cccc4oc5ccc(-c6ccc7ccccc7c6)cc5c34)nc(-c3ccc4c(ccc5ccccc54)c3)n2)cc1 ; c1ccc(-c2nc(-c3ccc4c(ccc5ccccc54)c3)nc(-c3cc(-c4ccc5ccccc5c4)cc4oc5ccccc5c34)n2)cc1]

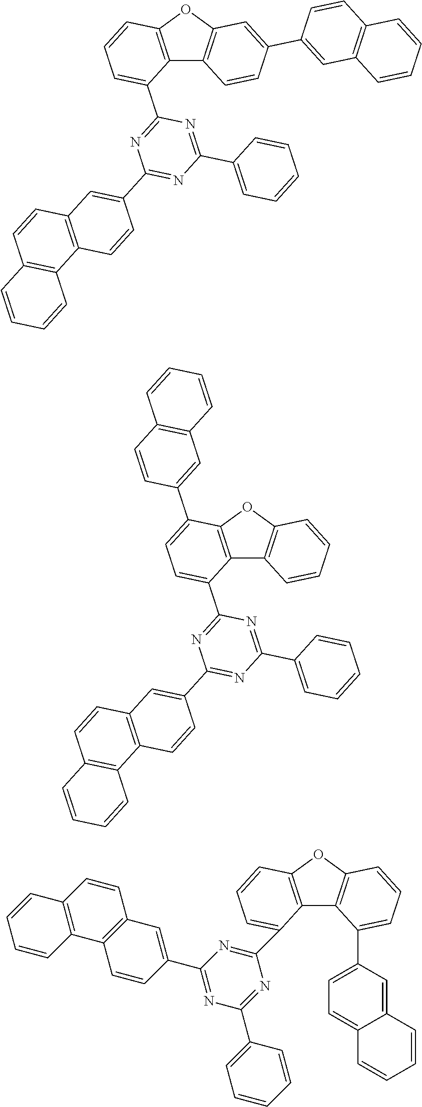 [SMILES: c1ccc(-c2nc(-c3ccc4c(ccc5ccccc54)c3)nc(-c3cccc4oc5cc(-c6ccc7ccccc7c6)ccc5c34)n2)cc1 ; c1ccc(-c2nc(-c3ccc4c(ccc5ccccc54)c3)nc(-c3ccc(-c4ccc5ccccc5c4)c4oc5ccccc5c34)n2)cc1 ; c1ccc(-c2nc(-c3ccc4c(ccc5ccccc54)c3)nc(-c3cccc4oc5cccc(-c6ccc7ccccc7c6)c5c34)n2)cc1]

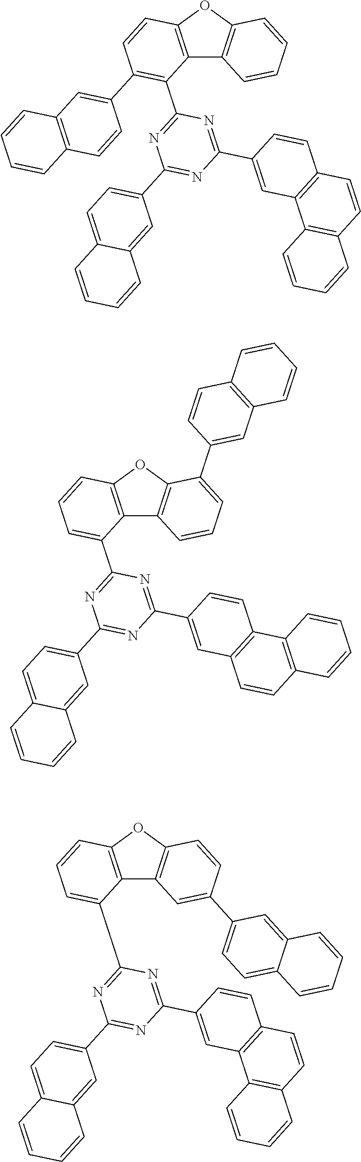 [SMILES: c1ccc2cc(-c3nc(-c4ccc5ccc6ccccc6c5c4)nc(-c4c(-c5ccc6ccccc6c5)ccc5oc6ccccc6c45)n3)ccc2c1 ; c1ccc2cc(-c3nc(-c4ccc5c(ccc6ccccc65)c4)nc(-c4cccc5oc6c(-c7ccc8ccccc8c7)cccc6c45)n3)ccc2c1 ; c1ccc2cc(-c3ccc4oc5cccc(Cc6nc(-c7ccc8ccccc8c7)nc(-c7ccc8ccc9ccccc9c8c7)n6)c5c4c3)ccc2c1]

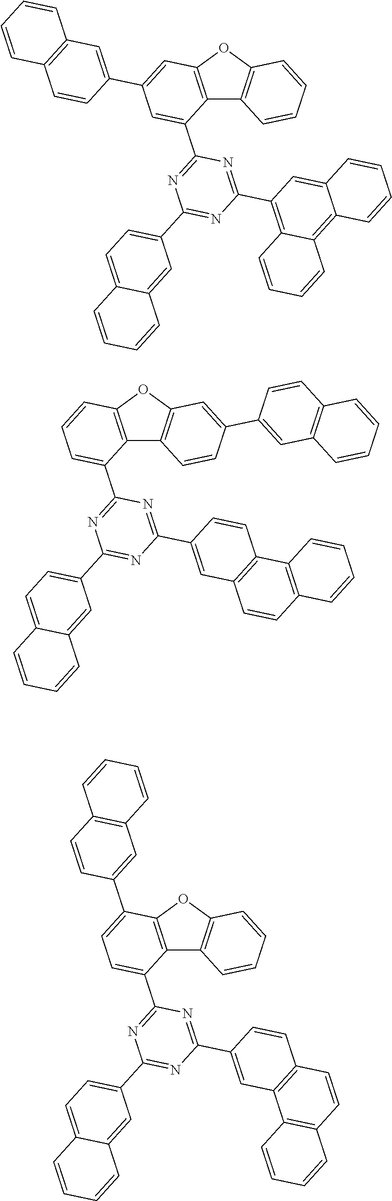

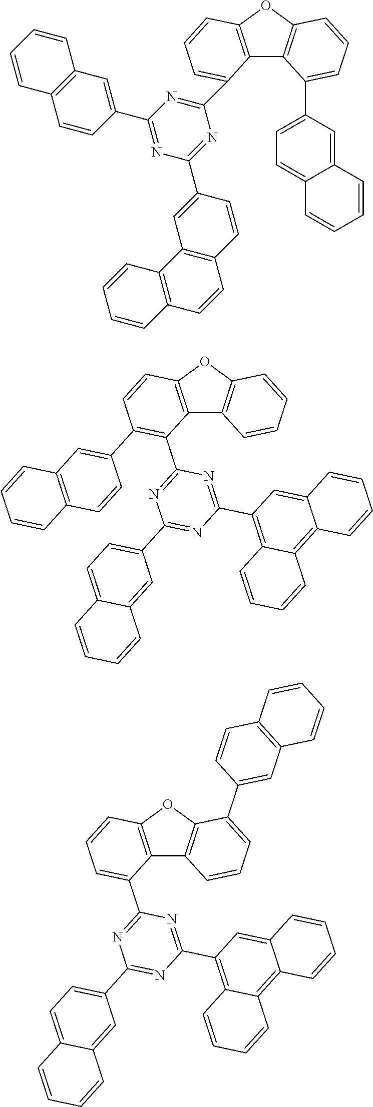

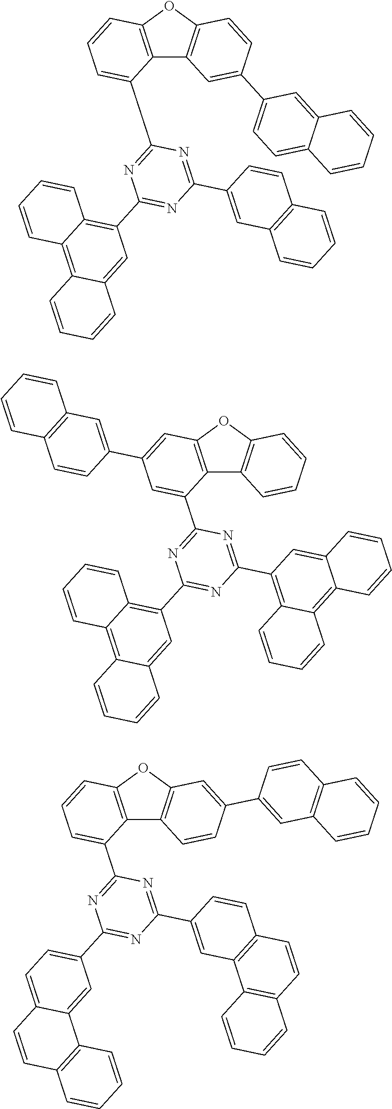

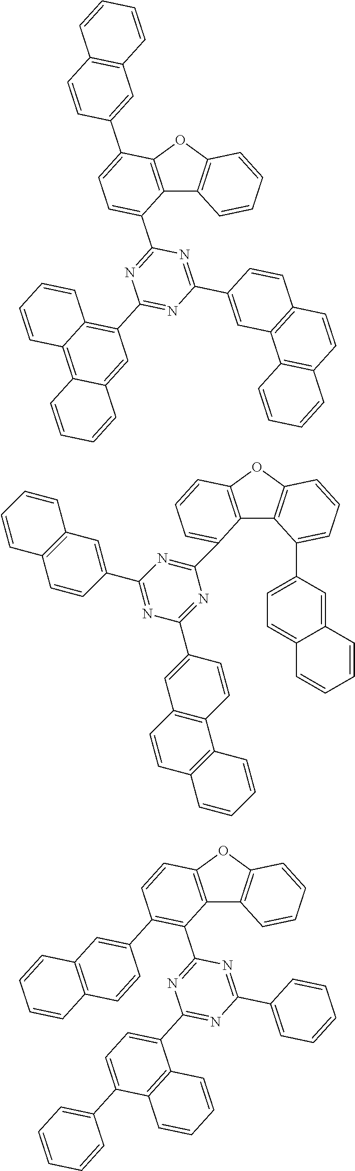

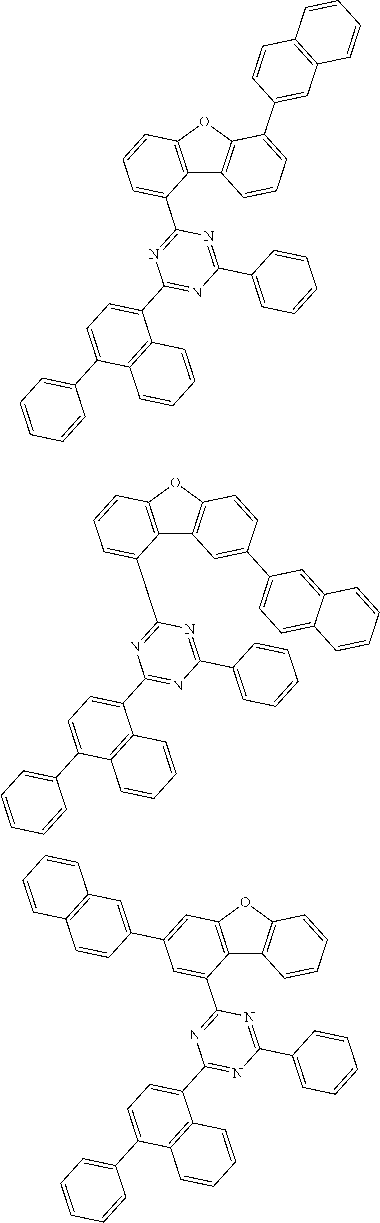

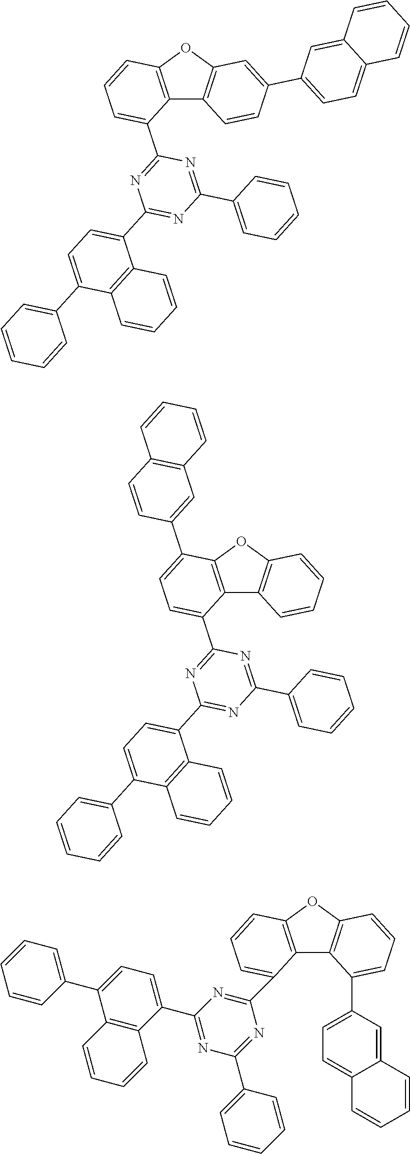 [SMILES: c1ccc(-c2nc(-c3ccc(-c4ccccc4)c4ccccc34)nc(-c3cccc4oc5cc(-c6ccc7ccccc7c6)ccc5c34)n2)cc1 ; c1ccc(-c2nc(-c3ccc(-c4ccccc4)c4ccccc34)nc(-c3ccc(-c4ccc5ccccc5c4)c4oc5ccccc5c34)n2)cc1 ; c1ccc(-c2nc(-c3ccc(-c4ccccc4)c4ccccc34)nc(-c3cccc4oc5cccc(-c6ccc7ccccc7c6)c5c34)n2)cc1]

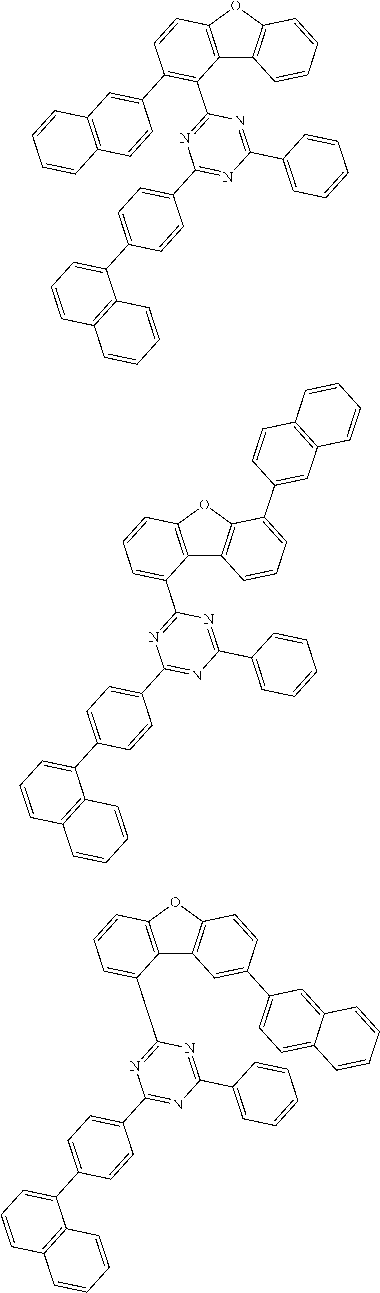 [SMILES: c1ccc(-c2nc(-c3ccc(-c4cccc5ccccc45)cc3)nc(-c3c(-c4ccc5ccccc5c4)ccc4oc5ccccc5c34)n2)cc1 ; c1ccc(-c2nc(-c3ccc(-c4cccc5ccccc45)cc3)nc(-c3cccc4oc5c(-c6ccc7ccccc7c6)cccc5c34)n2)cc1 ; c1ccc(-c2nc(Cc3cccc4oc5ccc(-c6ccc7ccccc7c6)cc5c34)nc(-c3ccc(-c4cccc5ccccc45)cc3)n2)cc1]

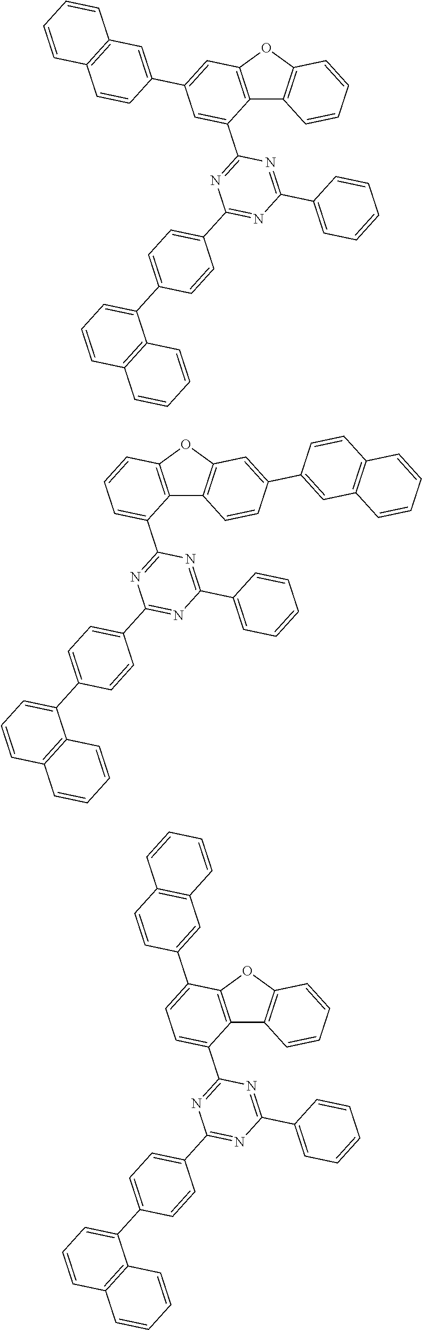

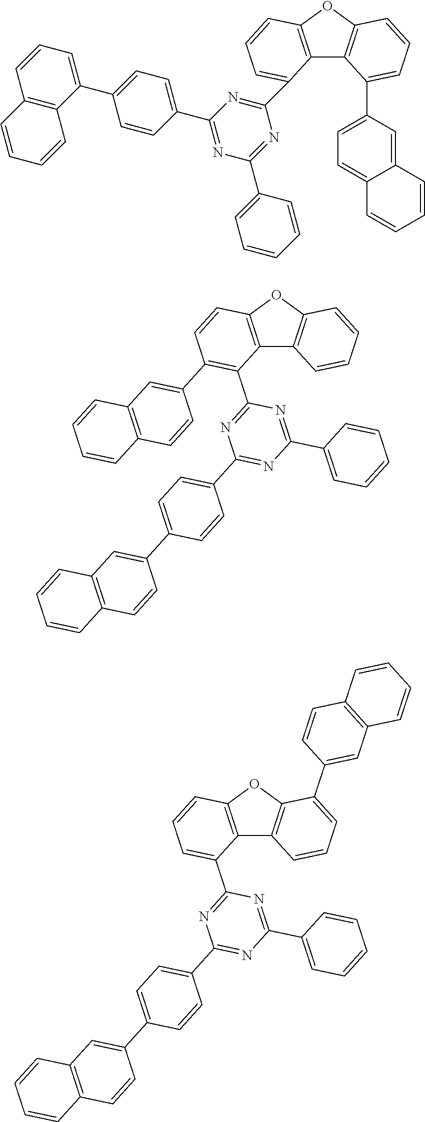

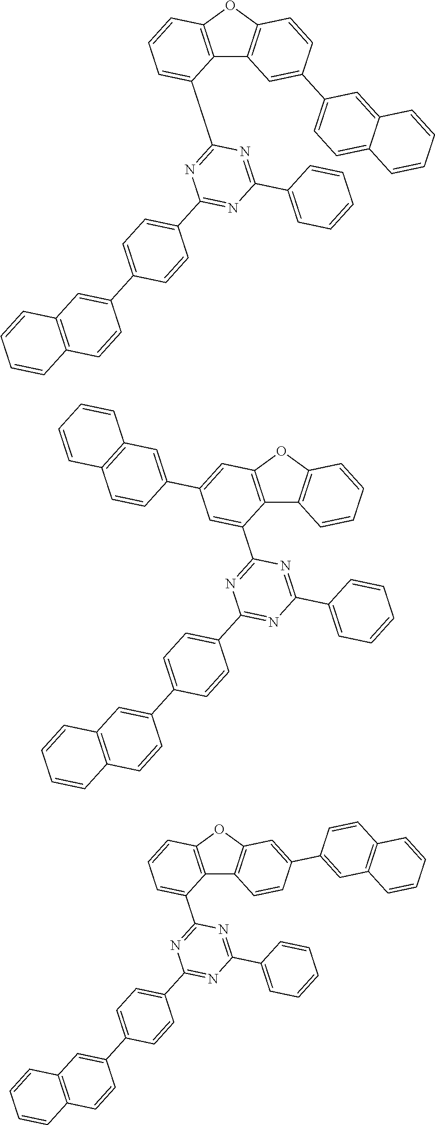 [SMILES: c1ccc(-c2nc(Cc3cccc4oc5ccc(-c6ccc7ccccc7c6)cc5c34)nc(-c3ccc(-c4ccc5ccccc5c4)cc3)n2)cc1 ; c1ccc(-c2nc(-c3ccc(-c4ccc5ccccc5c4)cc3)nc(-c3cc(-c4ccc5ccccc5c4)cc4oc5ccccc5c34)n2)cc1 ; c1ccc(-c2nc(-c3ccc(-c4ccc5ccccc5c4)cc3)nc(-c3cccc4oc5cc(-c6ccc7ccccc7c6)ccc5c34)n2)cc1]

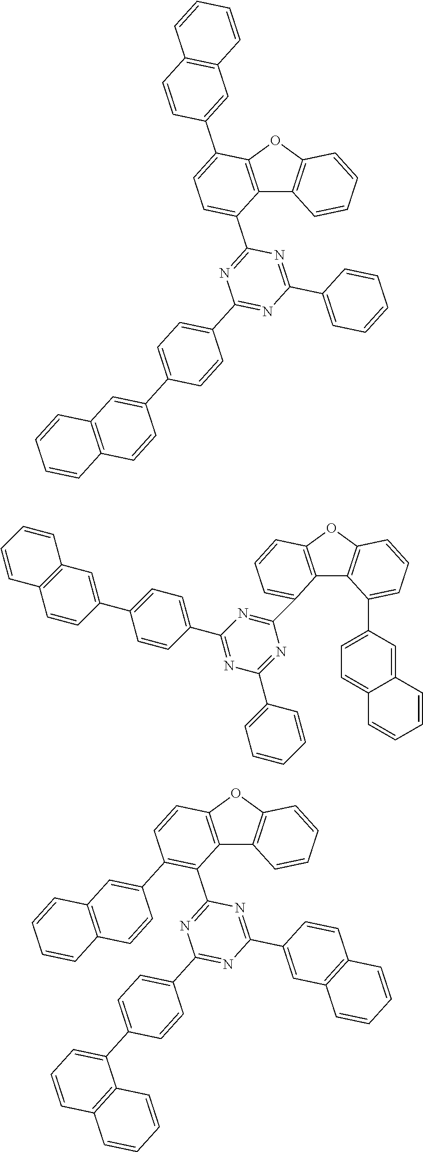

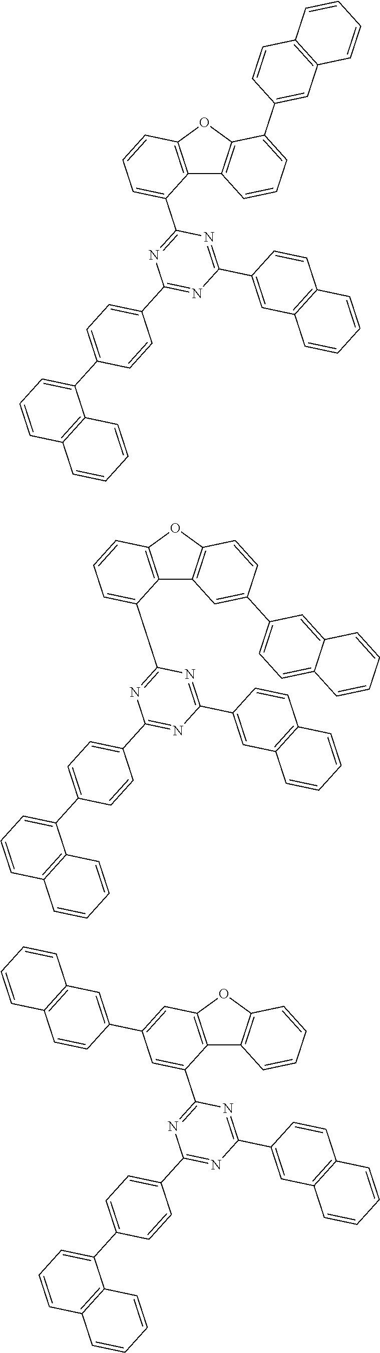

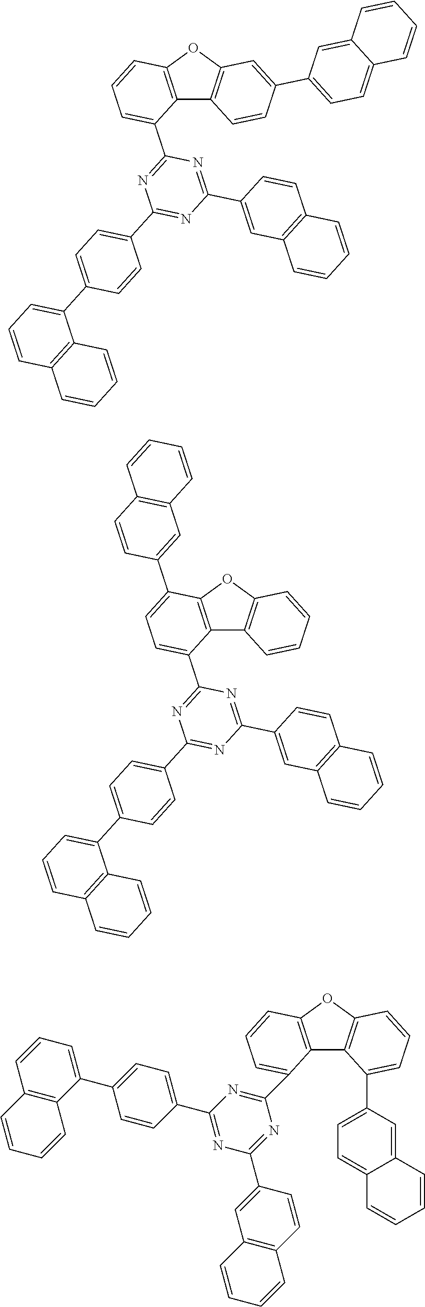 [SMILES: c1ccc2cc(-c3ccc4c(c3)oc3cccc(-c5nc(-c6ccc(-c7cccc8ccccc78)cc6)nc(-c6ccc7ccccc7c6)n5)c34)ccc2c1 ; c1ccc2cc(-c3nc(-c4ccc(-c5cccc6ccccc56)cc4)nc(-c4ccc(-c5ccc6ccccc6c5)c5oc6ccccc6c45)n3)ccc2c1 ; c1ccc2cc(-c3nc(-c4ccc(-c5cccc6ccccc56)cc4)nc(-c4cccc5oc6cccc(-c7ccc8ccccc8c7)c6c45)n3)ccc2c1]

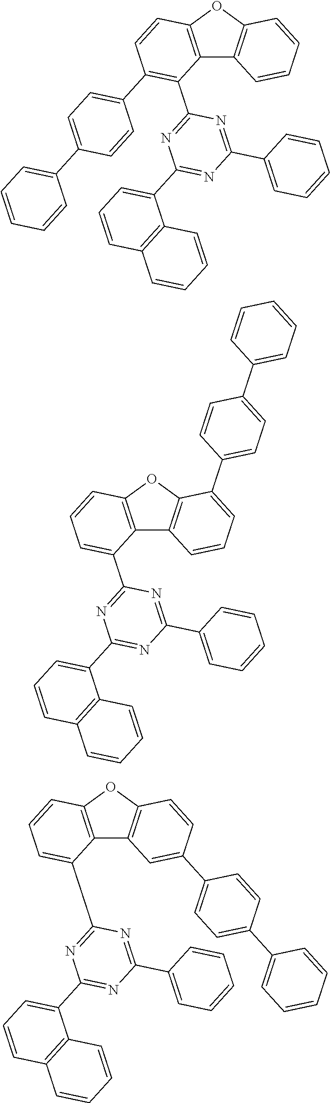

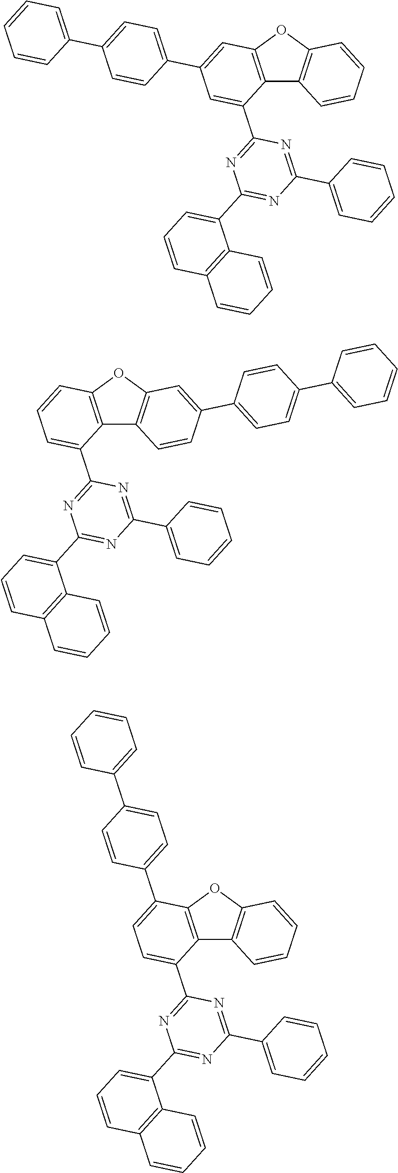 [SMILES: c1ccc(-c2ccc(-c3cc(-c4nc(-c5ccccc5)nc(-c5cccc6ccccc56)n4)c4c(c3)oc3ccccc34)cc2)cc1 ; c1ccc(-c2ccc(-c3ccc4c(c3)oc3cccc(-c5nc(-c6ccccc6)nc(-c6cccc7ccccc67)n5)c34)cc2)cc1 ; c1ccc(-c2ccc(-c3ccc(-c4nc(-c5ccccc5)nc(-c5cccc6ccccc56)n4)c4c3oc3ccccc34)cc2)cc1]

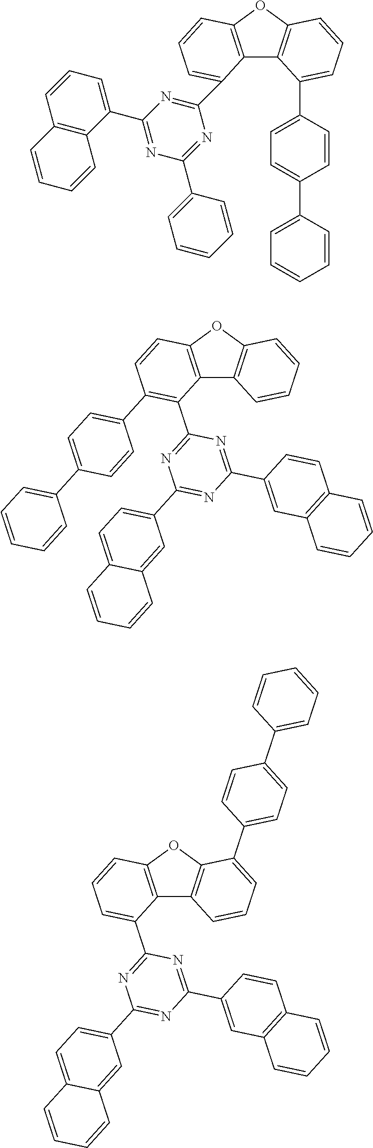 [SMILES: c1ccc(-c2ccc(-c3cccc4oc5cccc(-c6nc(-c7ccccc7)nc(-c7cccc8ccccc78)n6)c5c34)cc2)cc1 ; c1ccc(-c2ccc(-c3ccc4oc5ccccc5c4c3-c3nc(-c4ccc5ccccc5c4)nc(-c4ccc5ccccc5c4)n3)cc2)cc1 ; c1ccc(-c2ccc(-c3cccc4c3oc3cccc(-c5nc(-c6ccc7ccccc7c6)nc(-c6ccc7ccccc7c6)n5)c34)cc2)cc1]

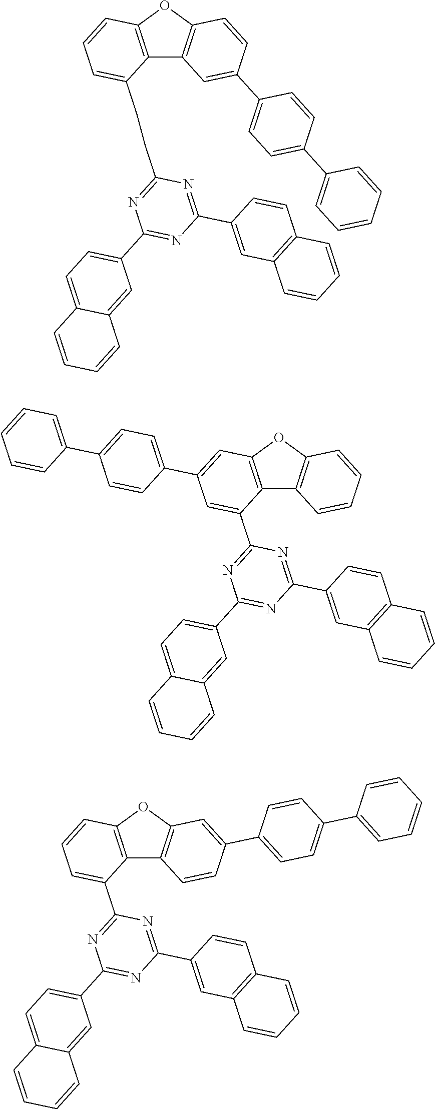 [SMILES: c1ccc(-c2ccc(-c3ccc4oc5cccc(CCc6nc(-c7ccc8ccccc8c7)nc(-c7ccc8ccccc8c7)n6)c5c4c3)cc2)cc1 ; c1ccc(-c2ccc(-c3cc(-c4nc(-c5ccc6ccccc6c5)nc(-c5ccc6ccccc6c5)n4)c4c(c3)oc3ccccc34)cc2)cc1 ; c1ccc(-c2ccc(-c3ccc4c(c3)oc3cccc(-c5nc(-c6ccc7ccccc7c6)nc(-c6ccc7ccccc7c6)n5)c34)cc2)cc1]

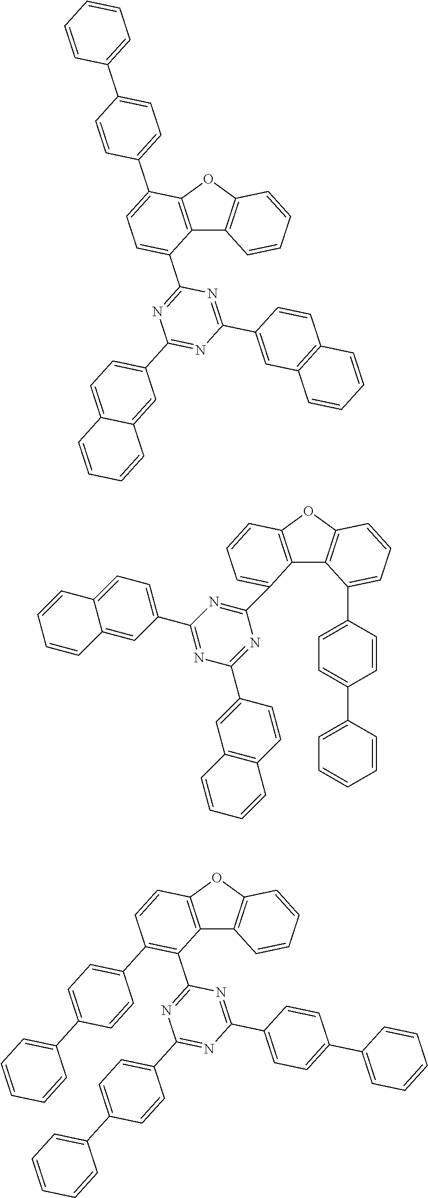 [SMILES: c1ccc(-c2ccc(-c3ccc(-c4nc(-c5ccc6ccccc6c5)nc(-c5ccc6ccccc6c5)n4)c4c3oc3ccccc34)cc2)cc1 ; c1ccc(-c2ccc(-c3cccc4oc5cccc(-c6nc(-c7ccc8ccccc8c7)nc(-c7ccc8ccccc8c7)n6)c5c34)cc2)cc1 ; c1ccc(-c2ccc(-c3nc(-c4ccc(-c5ccccc5)cc4)nc(-c4c(-c5ccc(-c6ccccc6)cc5)ccc5oc6ccccc6c45)n3)cc2)cc1]

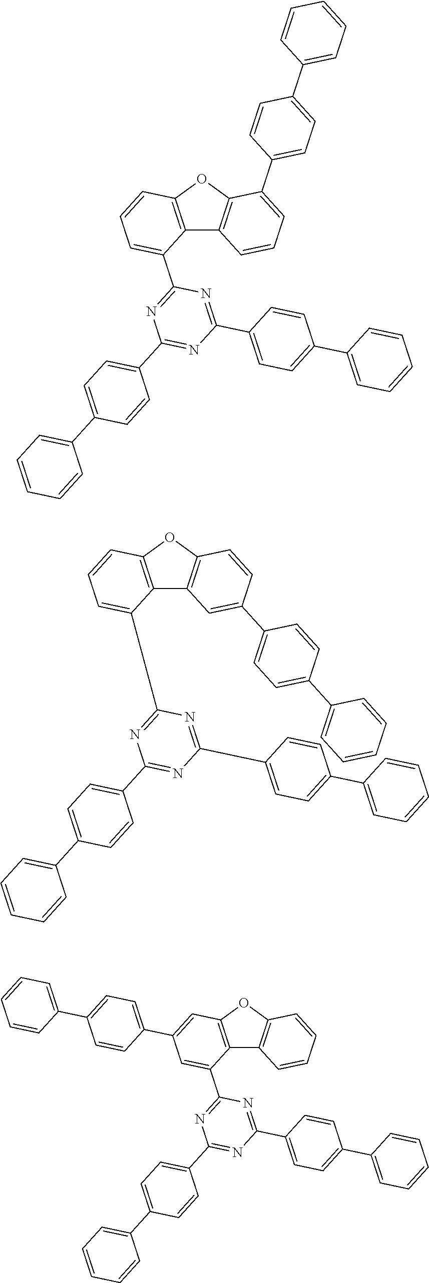 [SMILES: c1ccc(-c2ccc(-c3nc(-c4ccc(-c5ccccc5)cc4)nc(-c4cccc5oc6c(-c7ccc(-c8ccccc8)cc7)cccc6c45)n3)cc2)cc1 ; c1ccc(-c2ccc(Cc3nc(CCc4cccc5oc6ccc(-c7ccc(-c8ccccc8)cc7)cc6c45)nc(-c4ccc(-c5ccccc5)cc4)n3)cc2)cc1 ; c1ccc(-c2ccc(-c3cc(-c4nc(-c5ccc(-c6ccccc6)cc5)nc(-c5ccc(-c6ccccc6)cc5)n4)c4c(c3)oc3ccccc34)cc2)cc1]

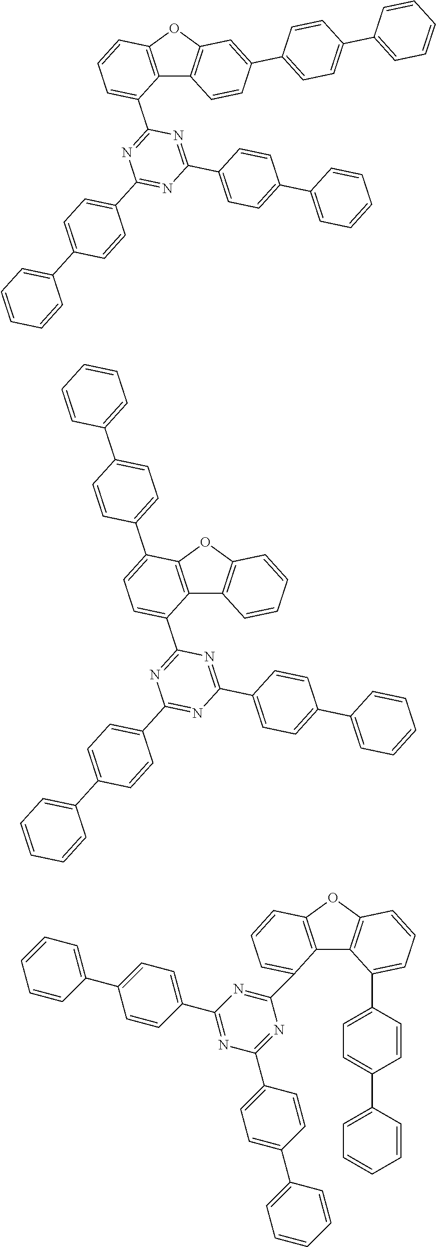 [SMILES: c1ccc(-c2ccc(-c3ccc4c(c3)oc3cccc(-c5nc(-c6ccc(-c7ccccc7)cc6)nc(-c6ccc(-c7ccccc7)cc6)n5)c34)cc2)cc1 ; c1ccc(-c2ccc(-c3nc(-c4ccc(-c5ccccc5)cc4)nc(-c4ccc(-c5ccc(-c6ccccc6)cc5)c5oc6ccccc6c45)n3)cc2)cc1 ; c1ccc(-c2ccc(-c3nc(-c4ccc(-c5ccccc5)cc4)nc(-c4cccc5oc6cccc(-c7ccc(-c8ccccc8)cc7)c6c45)n3)cc2)cc1]

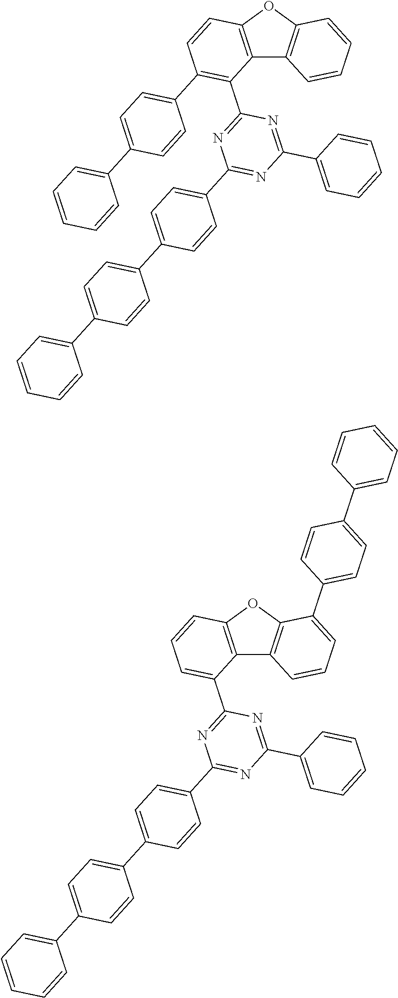 [SMILES: c1ccc(-c2ccc(-c3ccc(-c4nc(-c5ccccc5)nc(-c5c(-c6ccc(-c7ccccc7)cc6)ccc6oc7ccccc7c56)n4)cc3)cc2)cc1 ; c1ccc(-c2ccc(-c3ccc(-c4nc(-c5ccccc5)nc(-c5cccc6oc7c(-c8ccc(-c9ccccc9)cc8)cccc7c56)n4)cc3)cc2)cc1]

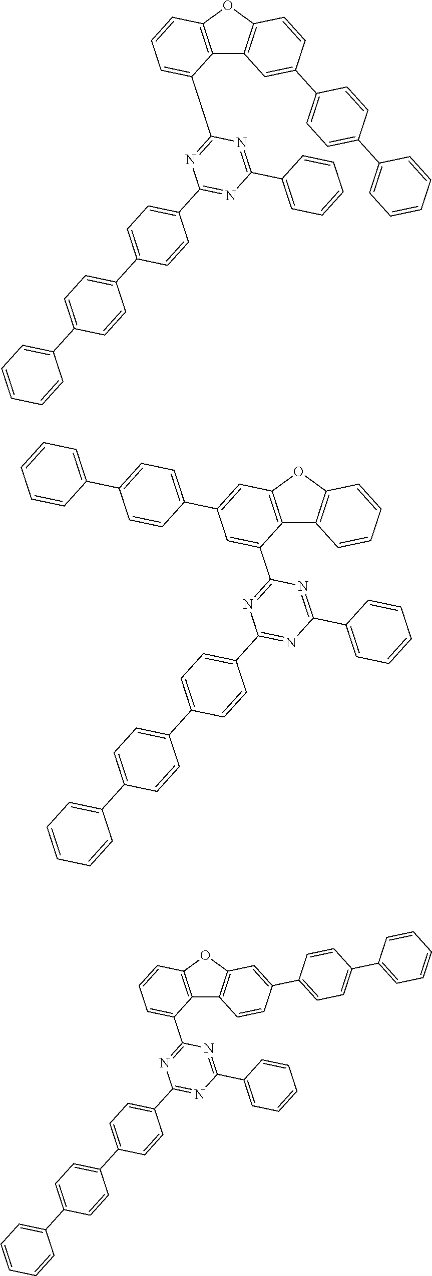

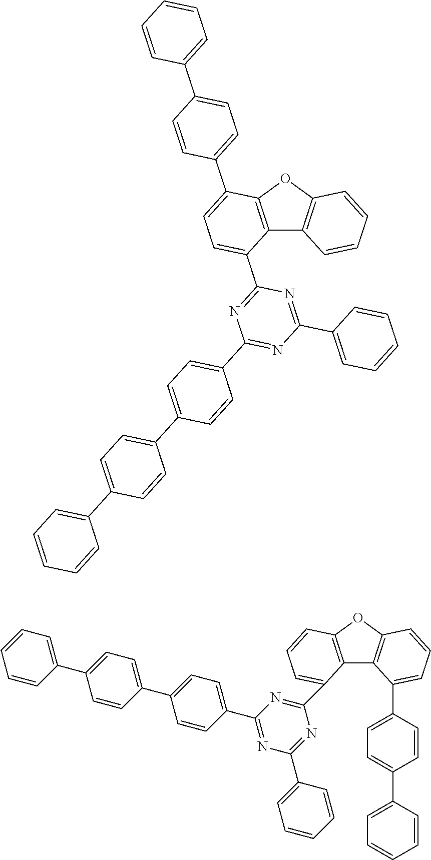 [SMILES: c1ccc(-c2ccc(-c3ccc(-c4nc(-c5ccccc5)nc(-c5ccc(-c6ccc(-c7ccccc7)cc6)c6oc7ccccc7c56)n4)cc3)cc2)cc1 ; c1ccc(-c2ccc(-c3ccc(-c4nc(-c5ccccc5)nc(-c5cccc6oc7cccc(-c8ccc(-c9ccccc9)cc8)c7c56)n4)cc3)cc2)cc1]

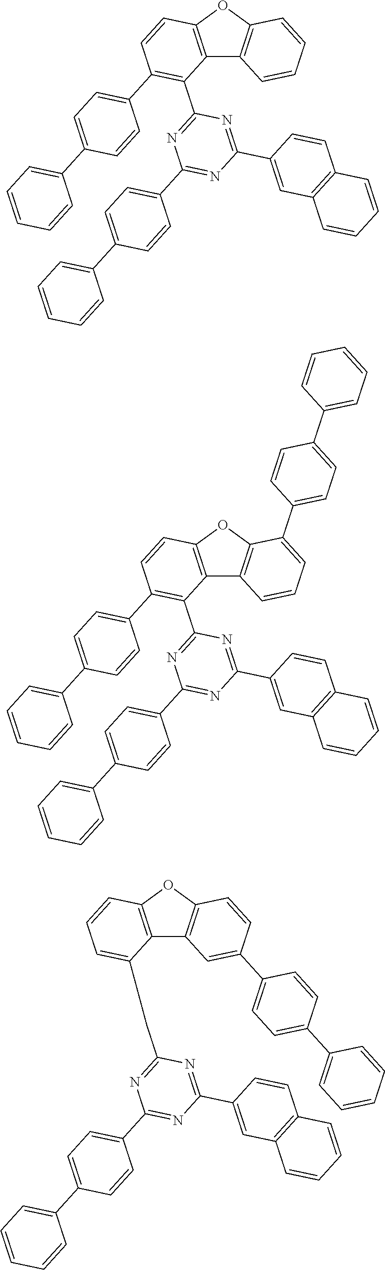 [SMILES: c1ccc(-c2ccc(-c3nc(-c4ccc5ccccc5c4)nc(-c4c(-c5ccc(-c6ccccc6)cc5)ccc5oc6ccccc6c45)n3)cc2)cc1 ; c1ccc(-c2ccc(-c3nc(-c4ccc5ccccc5c4)nc(-c4c(-c5ccc(-c6ccccc6)cc5)ccc5oc6c(-c7ccc(-c8ccccc8)cc7)cccc6c45)n3)cc2)cc1 ; c1ccc(-c2ccc(-c3ccc4oc5cccc(CCc6nc(-c7ccc(-c8ccccc8)cc7)nc(-c7ccc8ccccc8c7)n6)c5c4c3)cc2)cc1]

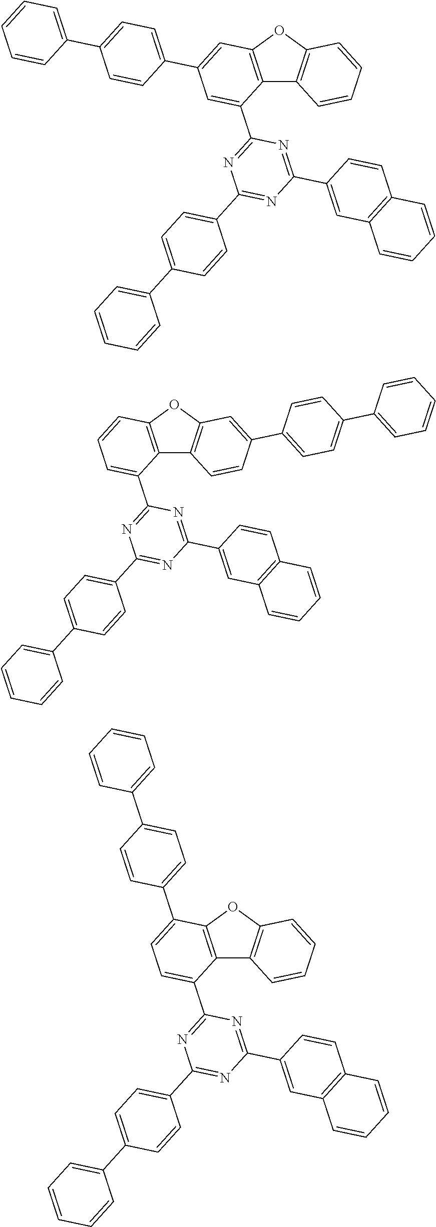 [SMILES: c1ccc(-c2ccc(-c3cc(-c4nc(-c5ccc(-c6ccccc6)cc5)nc(-c5ccc6ccccc6c5)n4)c4c(c3)oc3ccccc34)cc2)cc1 ; c1ccc(-c2ccc(-c3ccc4c(c3)oc3cccc(-c5nc(-c6ccc(-c7ccccc7)cc6)nc(-c6ccc7ccccc7c6)n5)c34)cc2)cc1 ; c1ccc(-c2ccc(-c3nc(-c4ccc5ccccc5c4)nc(-c4ccc(-c5ccc(-c6ccccc6)cc5)c5oc6ccccc6c45)n3)cc2)cc1]

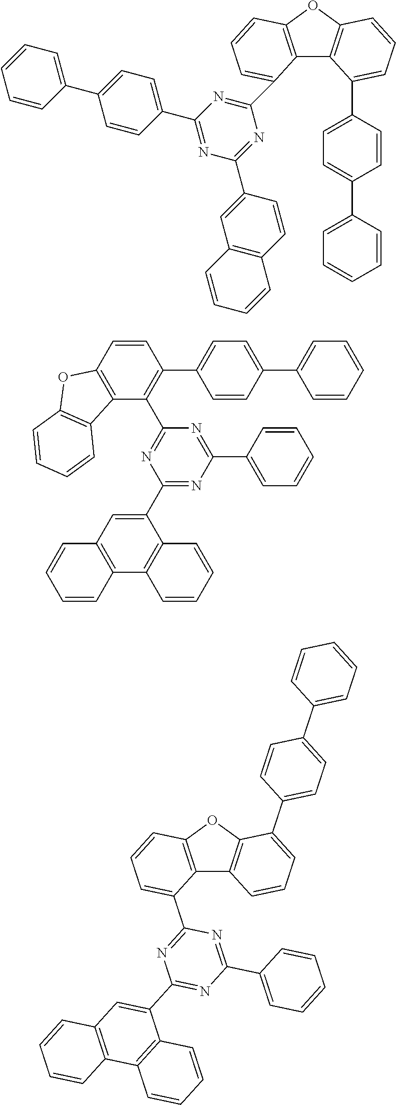

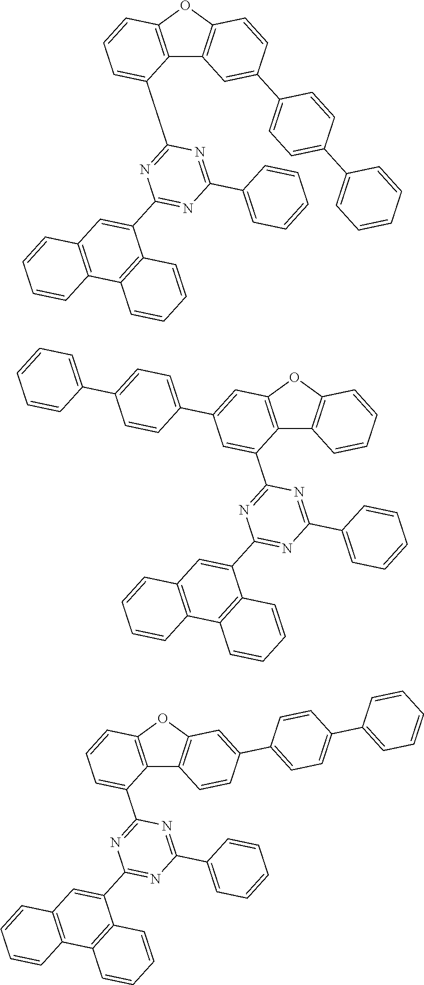 [SMILES: c1ccc(-c2ccc(-c3ccc4oc5cccc(Cc6nc(-c7ccccc7)nc(-c7cc8ccccc8c8ccccc78)n6)c5c4c3)cc2)cc1 ; c1ccc(-c2ccc(-c3cc(-c4nc(-c5ccccc5)nc(-c5cc6ccccc6c6ccccc56)n4)c4c(c3)oc3ccccc34)cc2)cc1 ; c1ccc(-c2ccc(-c3ccc4c(c3)oc3cccc(-c5nc(-c6ccccc6)nc(-c6cc7ccccc7c7ccccc67)n5)c34)cc2)cc1]

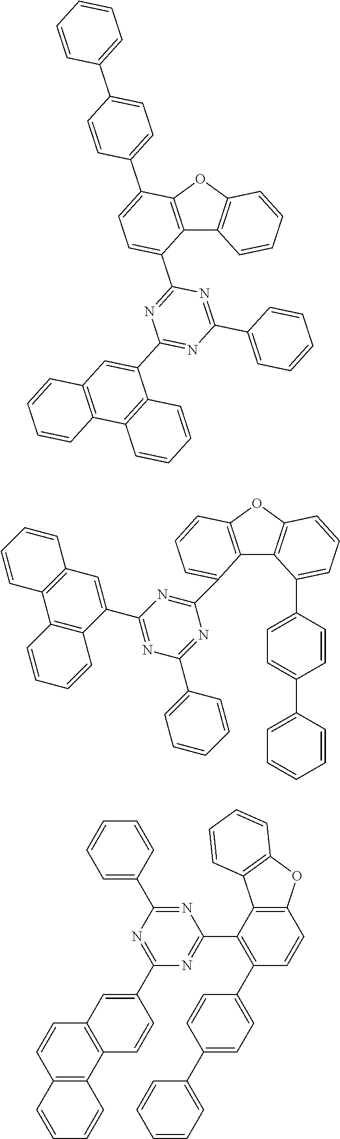 [SMILES: c1ccc(-c2ccc(-c3ccc(-c4nc(-c5ccccc5)nc(-c5cc6ccccc6c6ccccc56)n4)c4c3oc3ccccc34)cc2)cc1 ; c1ccc(-c2ccc(-c3cccc4oc5cccc(-c6nc(-c7ccccc7)nc(-c7cc8ccccc8c8ccccc78)n6)c5c34)cc2)cc1 ; c1ccc(-c2ccc(-c3ccc4oc5ccccc5c4c3-c3nc(-c4ccccc4)nc(-c4ccc5c(ccc6ccccc65)c4)n3)cc2)cc1]

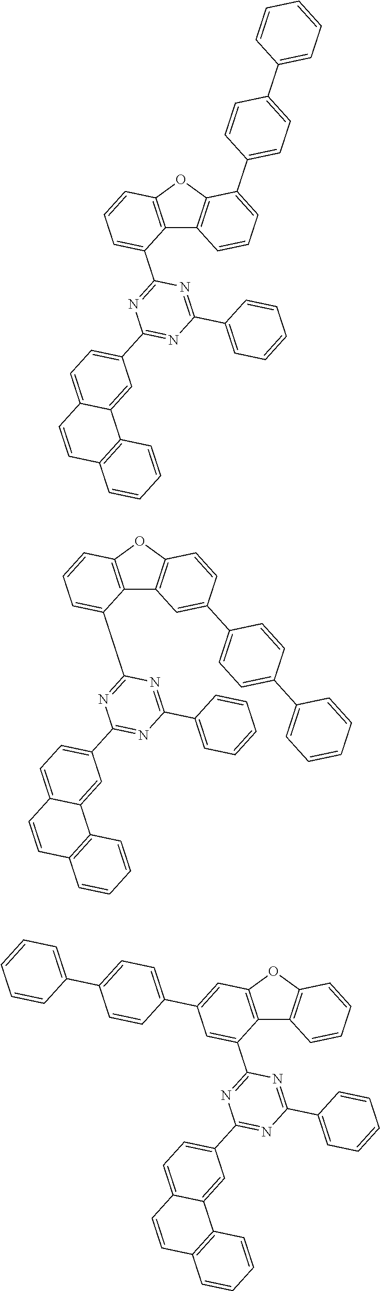

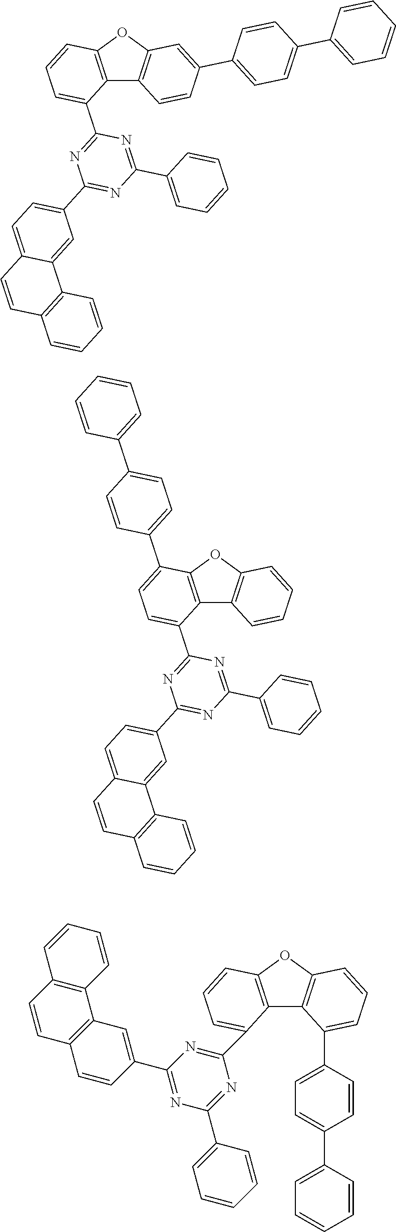

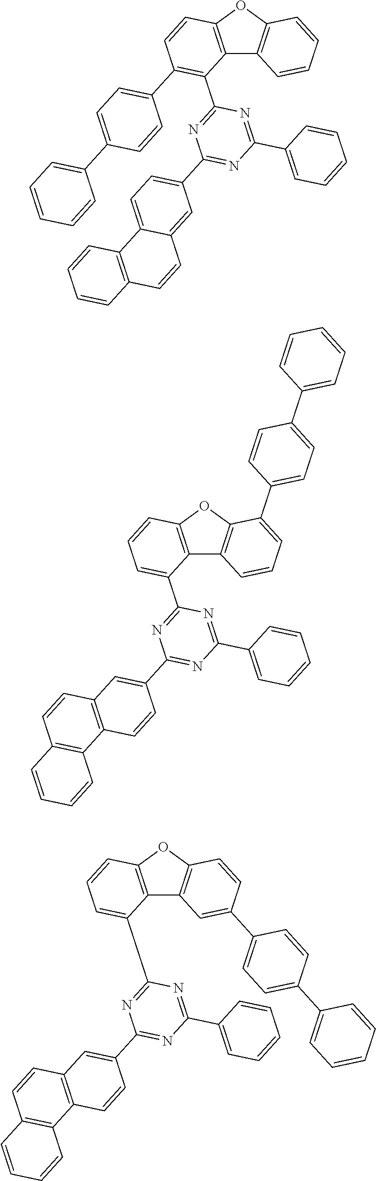 [SMILES: c1ccc(-c2ccc(-c3ccc4oc5ccccc5c4c3-c3nc(-c4ccccc4)nc(-c4ccc5c(ccc6ccccc65)c4)n3)cc2)cc1 ; c1ccc(-c2ccc(-c3cccc4c3oc3cccc(-c5nc(-c6ccccc6)nc(-c6ccc7c(ccc8ccccc87)c6)n5)c34)cc2)cc1 ; c1ccc(-c2ccc(-c3ccc4oc5cccc(Cc6nc(-c7ccccc7)nc(-c7ccc8c(ccc9ccccc98)c7)n6)c5c4c3)cc2)cc1]

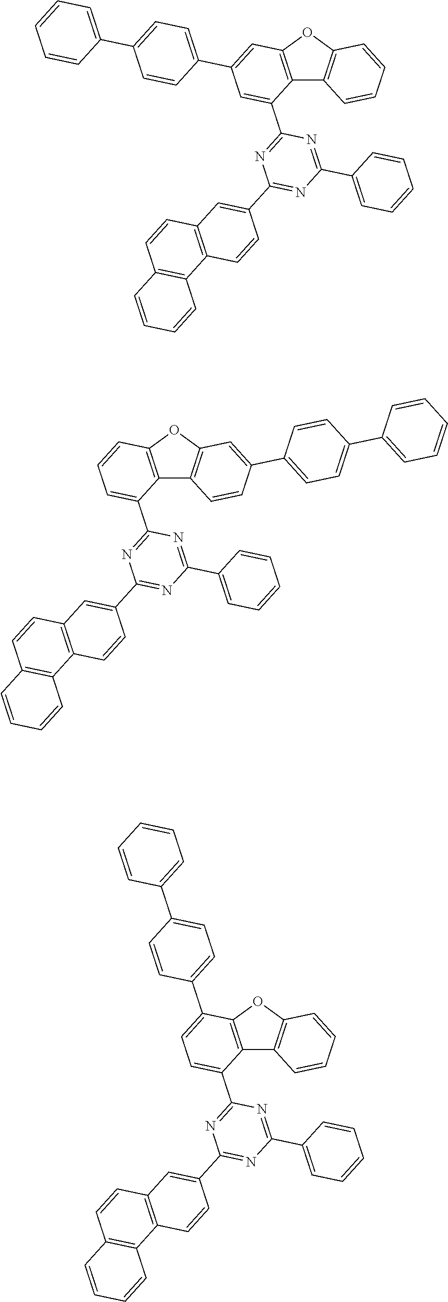 [SMILES: c1ccc(-c2ccc(-c3cc(-c4nc(-c5ccccc5)nc(-c5ccc6c(ccc7ccccc76)c5)n4)c4c(c3)oc3ccccc34)cc2)cc1 ; c1ccc(-c2ccc(-c3ccc4c(c3)oc3cccc(-c5nc(-c6ccccc6)nc(-c6ccc7c(ccc8ccccc87)c6)n5)c34)cc2)cc1 ; c1ccc(-c2ccc(-c3ccc(-c4nc(-c5ccccc5)nc(-c5ccc6c(ccc7ccccc76)c5)n4)c4c3oc3ccccc34)cc2)cc1]

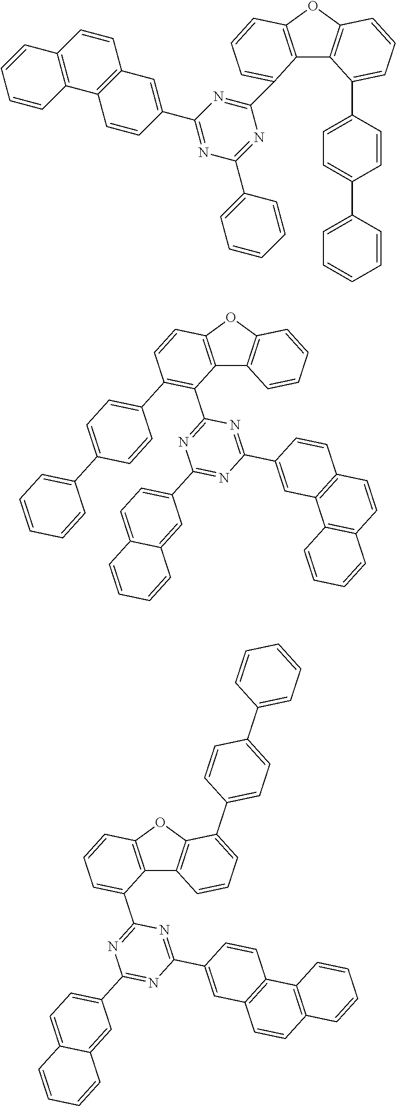 [SMILES: c1ccc(-c2ccc(-c3cccc4oc5cccc(-c6nc(-c7ccccc7)nc(-c7ccc8c(ccc9ccccc98)c7)n6)c5c34)cc2)cc1 ; c1ccc(-c2ccc(-c3ccc4oc5ccccc5c4c3-c3nc(-c4ccc5ccccc5c4)nc(-c4ccc5ccc6ccccc6c5c4)n3)cc2)cc1 ; c1ccc(-c2ccc(-c3cccc4c3oc3cccc(-c5nc(-c6ccc7ccccc7c6)nc(-c6ccc7c(ccc8ccccc87)c6)n5)c34)cc2)cc1]

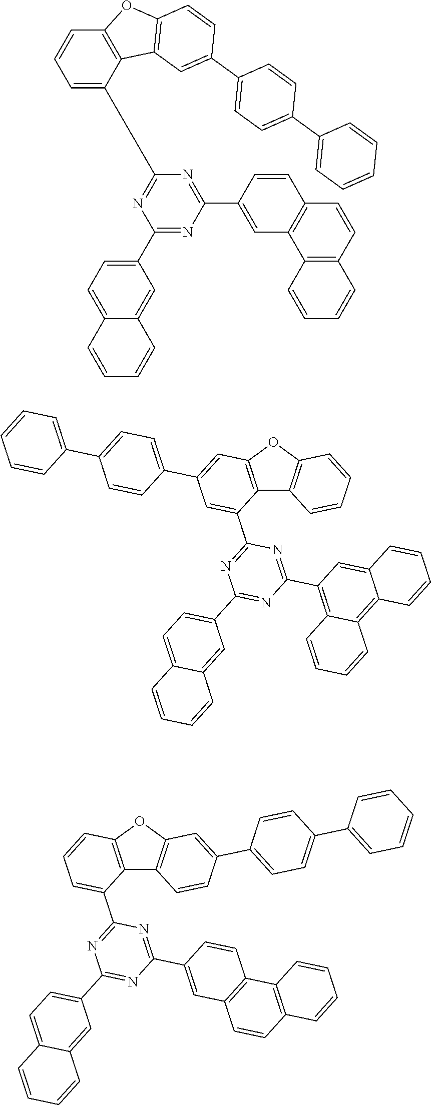 [SMILES: c1ccc(-c2ccc(-c3ccc4oc5cccc(CCc6nc(-c7ccc8ccccc8c7)nc(-c7ccc8ccc9ccccc9c8c7)n6)c5c4c3)cc2)cc1 ; c1ccc(-c2ccc(-c3cc(-c4nc(-c5ccc6ccccc6c5)nc(-c5cc6ccccc6c6ccccc56)n4)c4c(c3)oc3ccccc34)cc2)cc1 ; c1ccc(-c2ccc(-c3ccc4c(c3)oc3cccc(-c5nc(-c6ccc7ccccc7c6)nc(-c6ccc7c(ccc8ccccc87)c6)n5)c34)cc2)cc1]

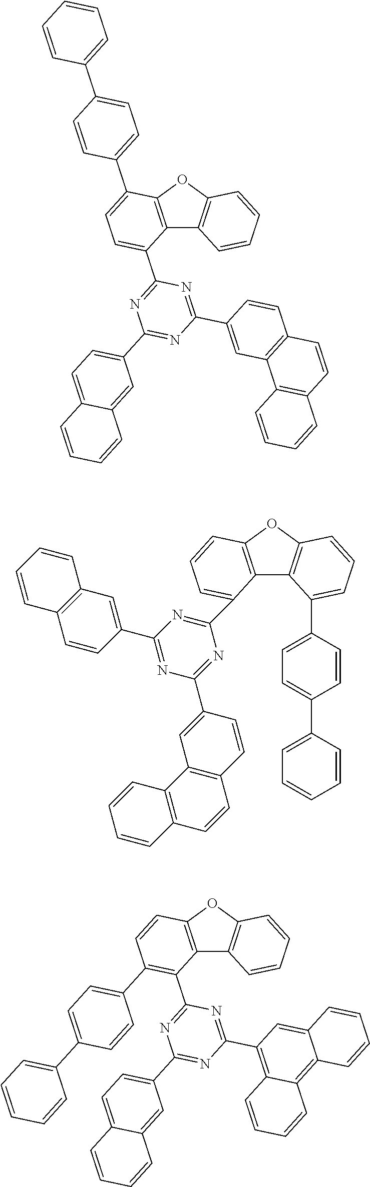 [SMILES: c1ccc(-c2ccc(-c3ccc(-c4nc(-c5ccc6ccccc6c5)nc(-c5ccc6ccc7ccccc7c6c5)n4)c4c3oc3ccccc34)cc2)cc1 ; c1ccc(-c2ccc(-c3cccc4oc5cccc(-c6nc(-c7ccc8ccccc8c7)nc(-c7ccc8ccc9ccccc9c8c7)n6)c5c34)cc2)cc1 ; c1ccc(-c2ccc(-c3ccc4oc5ccccc5c4c3-c3nc(-c4ccc5ccccc5c4)nc(-c4cc5ccccc5c5ccccc45)n3)cc2)cc1]

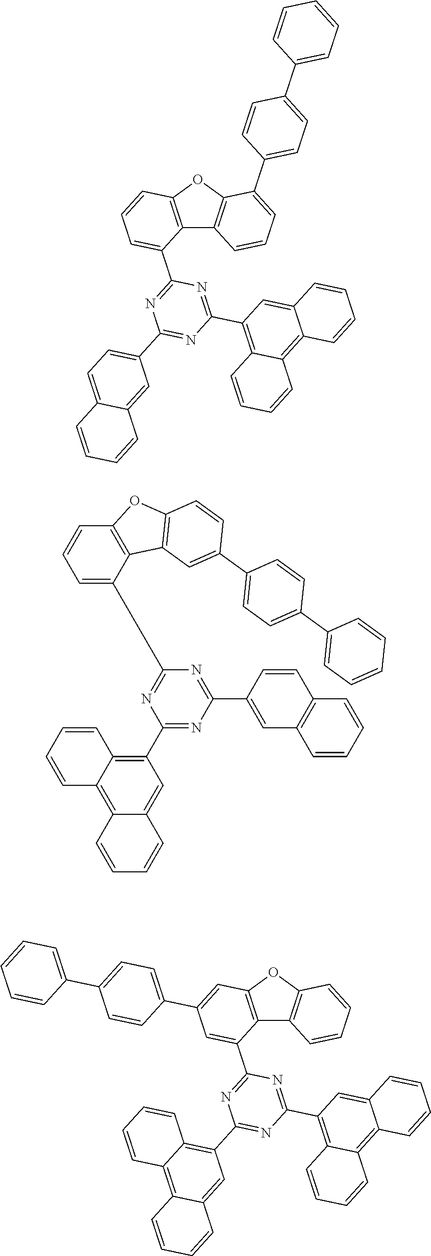

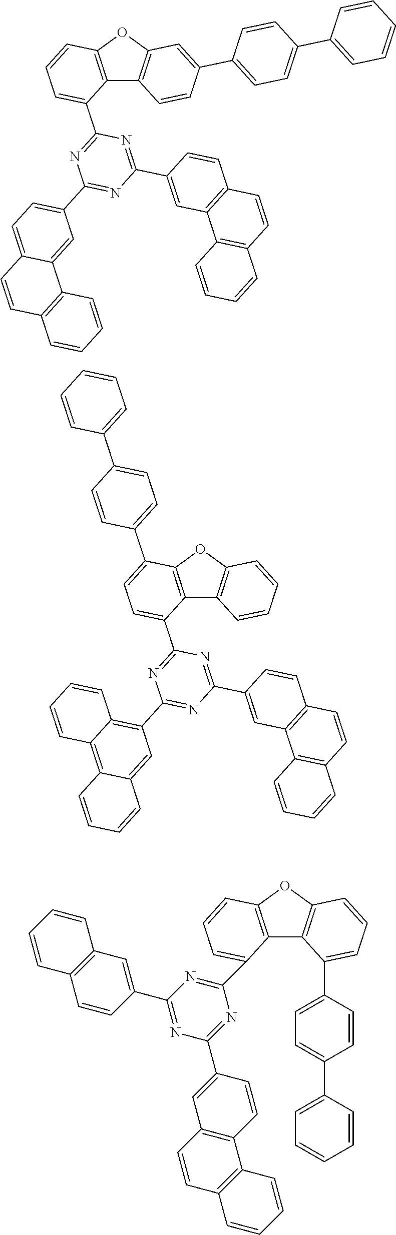

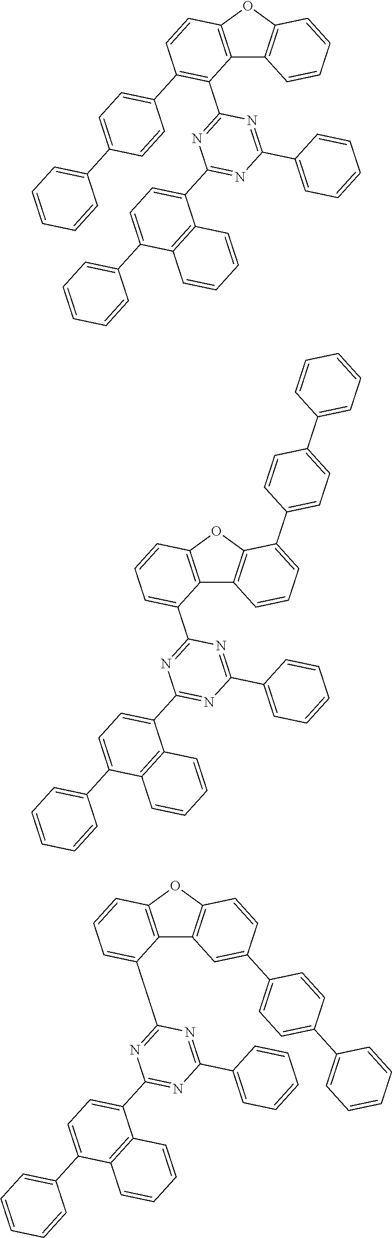 [SMILES: c1ccc(-c2ccc(-c3ccc4oc5ccccc5c4c3-c3nc(-c4ccccc4)nc(-c4ccc(-c5ccccc5)c5ccccc45)n3)cc2)cc1 ; c1ccc(-c2ccc(-c3cccc4c3oc3cccc(-c5nc(-c6ccccc6)nc(-c6ccc(-c7ccccc7)c7ccccc67)n5)c34)cc2)cc1 ; c1ccc(-c2ccc(-c3ccc4oc5cccc(Cc6nc(-c7ccccc7)nc(-c7ccc(-c8ccccc8)c8ccccc78)n6)c5c4c3)cc2)cc1]

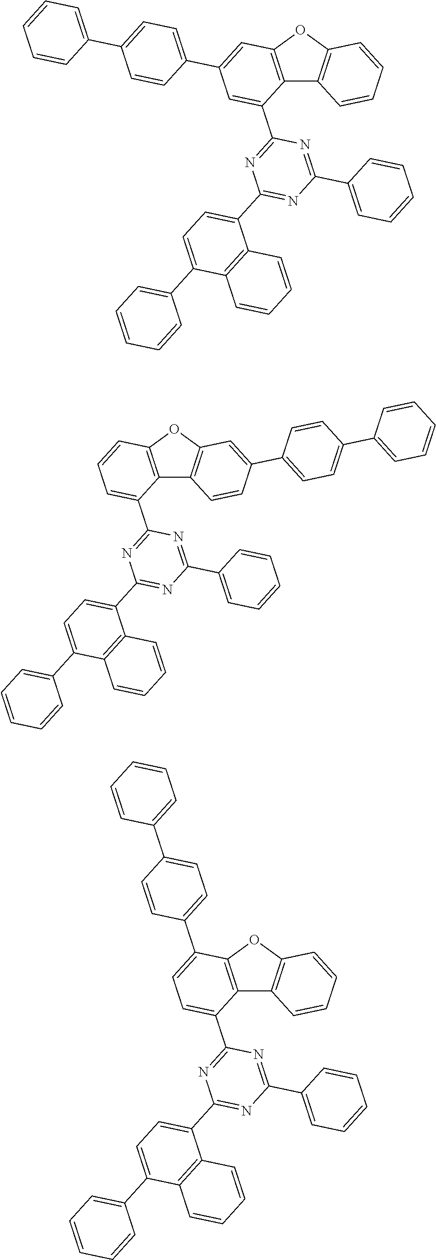 [SMILES: c1ccc(-c2ccc(-c3cc(-c4nc(-c5ccccc5)nc(-c5ccc(-c6ccccc6)c6ccccc56)n4)c4c(c3)oc3ccccc34)cc2)cc1 ; c1ccc(-c2ccc(-c3ccc4c(c3)oc3cccc(-c5nc(-c6ccccc6)nc(-c6ccc(-c7ccccc7)c7ccccc67)n5)c34)cc2)cc1 ; c1ccc(-c2ccc(-c3ccc(-c4nc(-c5ccccc5)nc(-c5ccc(-c6ccccc6)c6ccccc56)n4)c4c3oc3ccccc34)cc2)cc1]

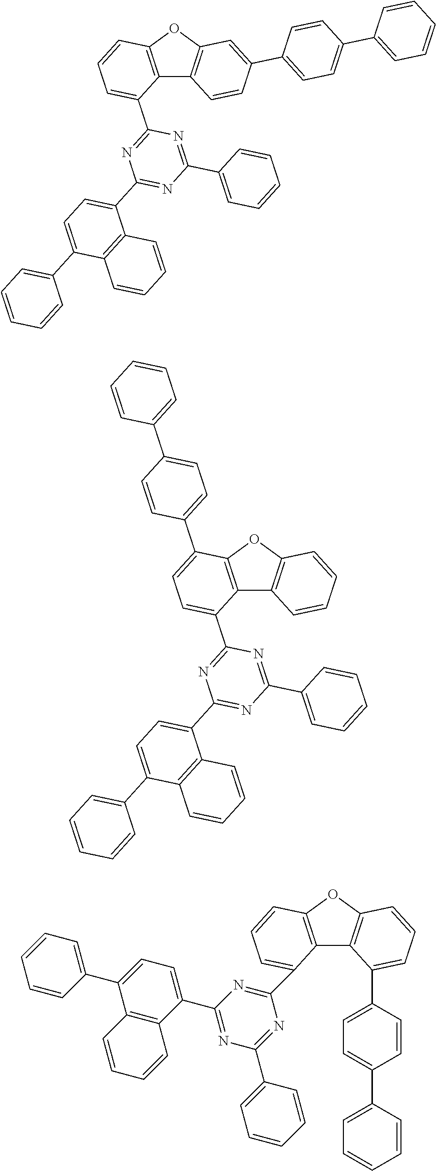 [SMILES: c1ccc(-c2ccc(-c3ccc4c(c3)oc3cccc(-c5nc(-c6ccccc6)nc(-c6ccc(-c7ccccc7)c7ccccc67)n5)c34)cc2)cc1 ; c1ccc(-c2ccc(-c3ccc(-c4nc(-c5ccccc5)nc(-c5ccc(-c6ccccc6)c6ccccc56)n4)c4c3oc3ccccc34)cc2)cc1 ; c1ccc(-c2ccc(-c3cccc4oc5cccc(-c6nc(-c7ccccc7)nc(-c7ccc(-c8ccccc8)c8ccccc78)n6)c5c34)cc2)cc1]

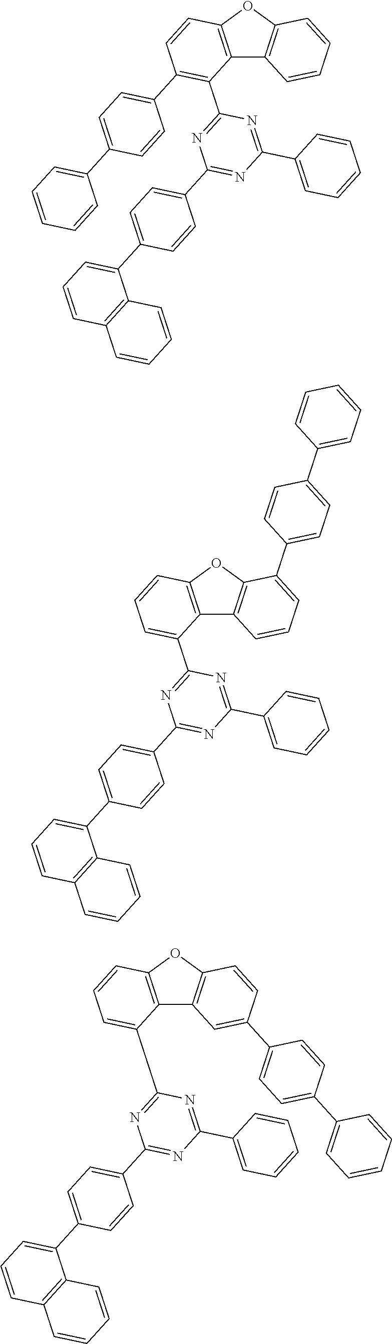

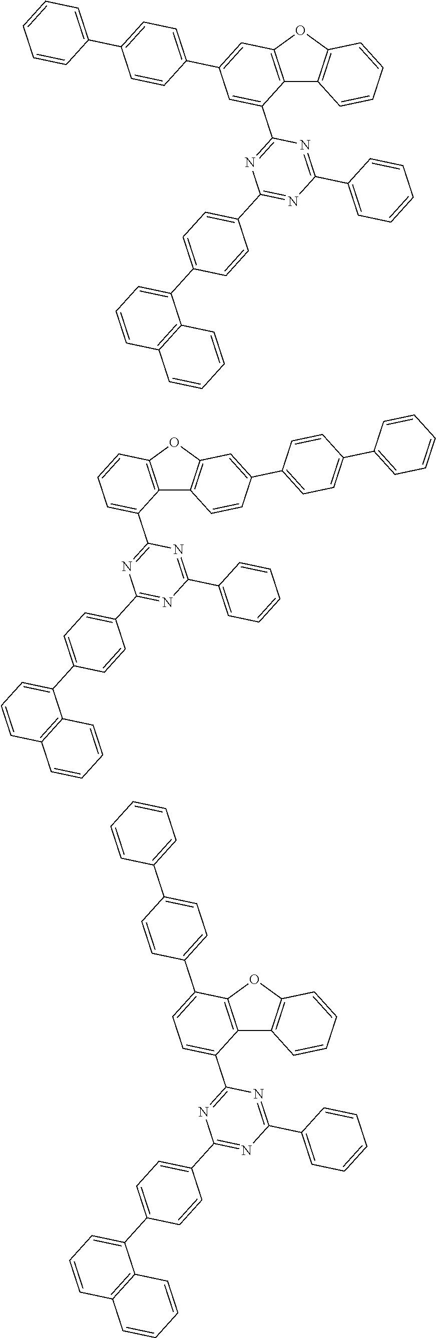 [SMILES: c1ccc(-c2ccc(-c3cc(-c4nc(-c5ccccc5)nc(-c5ccc(-c6cccc7ccccc67)cc5)n4)c4c(c3)oc3ccccc34)cc2)cc1 ; c1ccc(-c2ccc(-c3ccc4c(c3)oc3cccc(-c5nc(-c6ccccc6)nc(-c6ccc(-c7cccc8ccccc78)cc6)n5)c34)cc2)cc1 ; c1ccc(-c2ccc(-c3ccc(-c4nc(-c5ccccc5)nc(-c5ccc(-c6cccc7ccccc67)cc5)n4)c4c3oc3ccccc34)cc2)cc1]

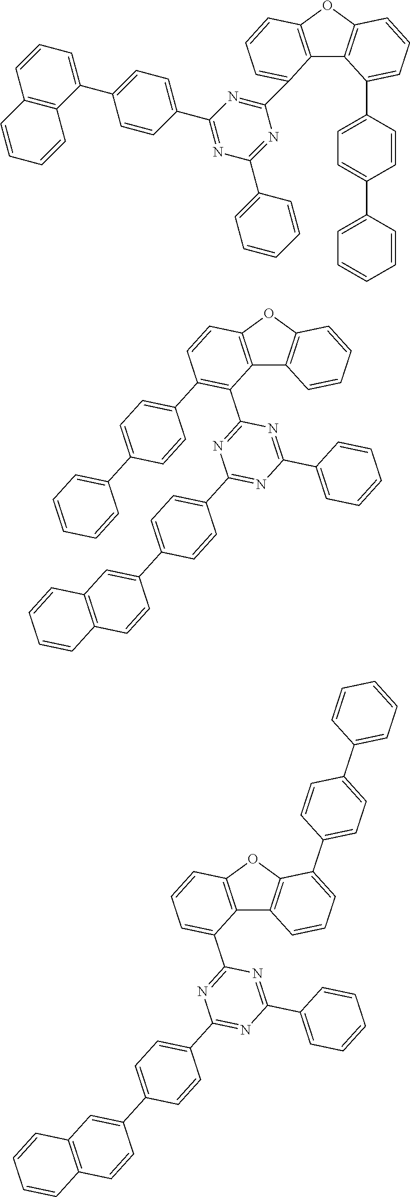 [SMILES: c1ccc(-c2ccc(-c3cccc4oc5cccc(-c6nc(-c7ccccc7)nc(-c7ccc(-c8cccc9ccccc89)cc7)n6)c5c34)cc2)cc1 ; c1ccc(-c2ccc(-c3ccc4oc5ccccc5c4c3-c3nc(-c4ccccc4)nc(-c4ccc(-c5ccc6ccccc6c5)cc4)n3)cc2)cc1 ; c1ccc(-c2ccc(-c3cccc4c3oc3cccc(-c5nc(-c6ccccc6)nc(-c6ccc(-c7ccc8ccccc8c7)cc6)n5)c34)cc2)cc1]

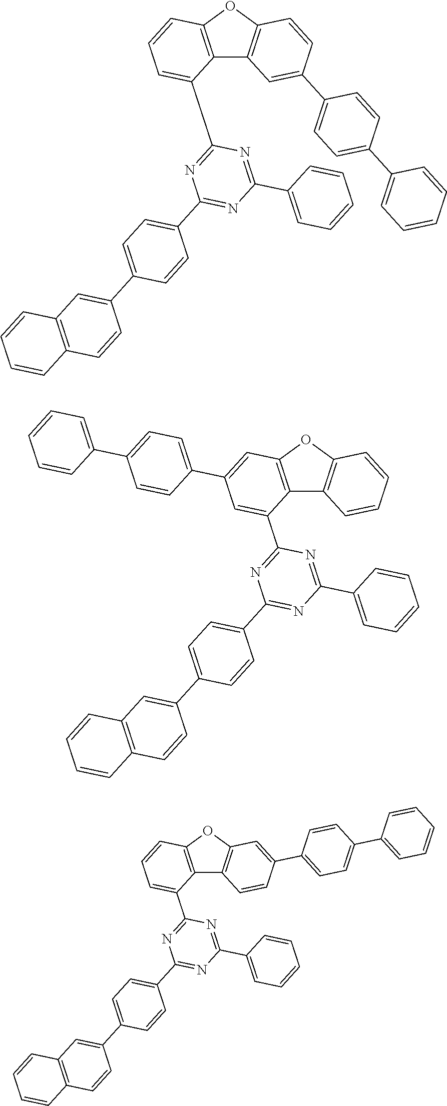

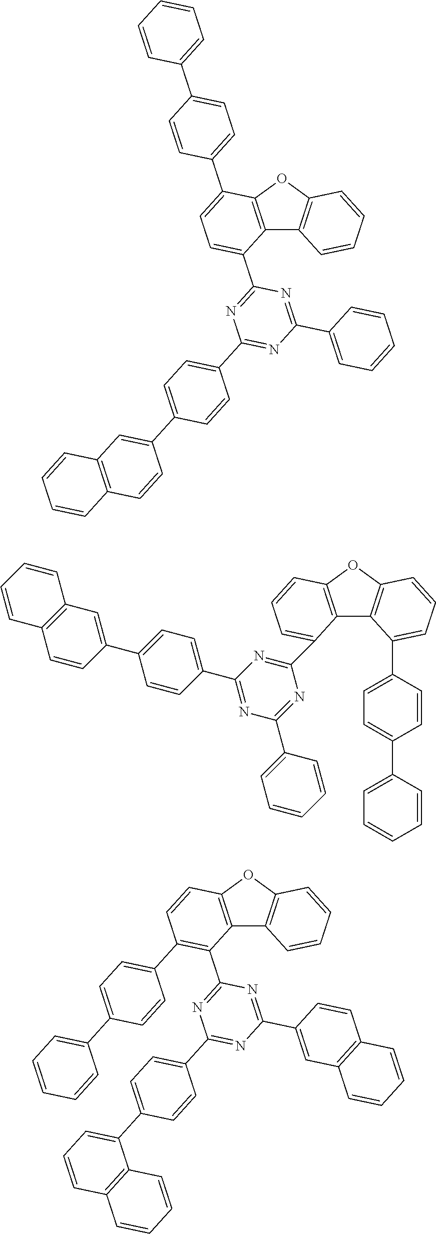 [SMILES: c1ccc(-c2ccc(-c3ccc(-c4nc(-c5ccccc5)nc(-c5ccc(-c6ccc7ccccc7c6)cc5)n4)c4c3oc3ccccc34)cc2)cc1 ; c1ccc(-c2ccc(-c3cccc4oc5cccc(-c6nc(-c7ccccc7)nc(-c7ccc(-c8ccc9ccccc9c8)cc7)n6)c5c34)cc2)cc1 ; c1ccc(-c2ccc(-c3ccc4oc5ccccc5c4c3-c3nc(-c4ccc(-c5cccc6ccccc56)cc4)nc(-c4ccc5ccccc5c4)n3)cc2)cc1]

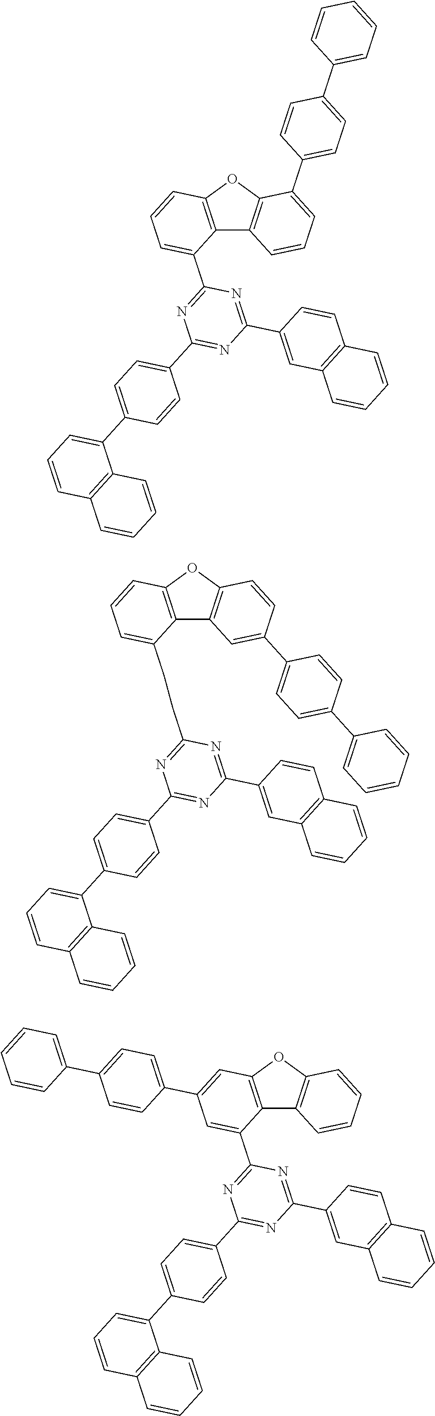

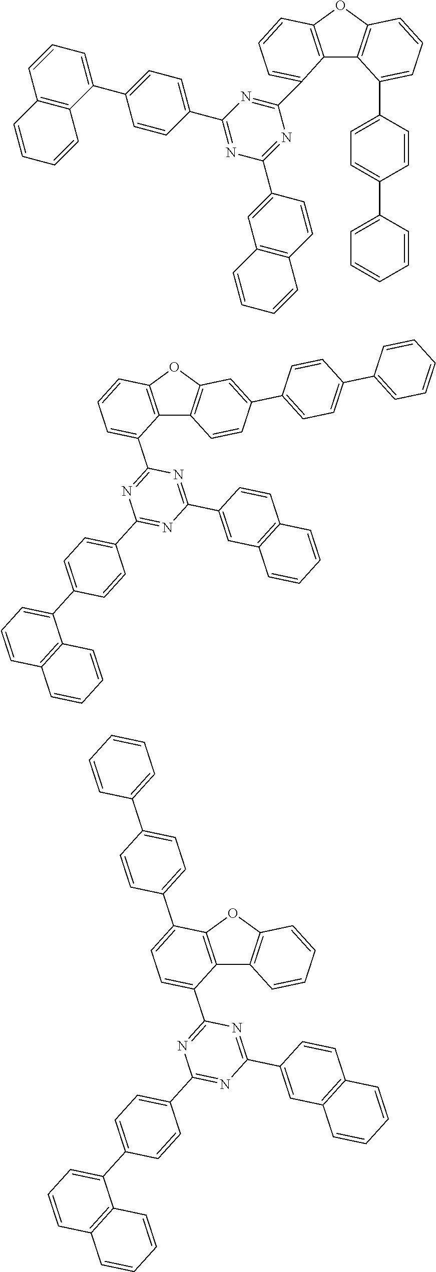

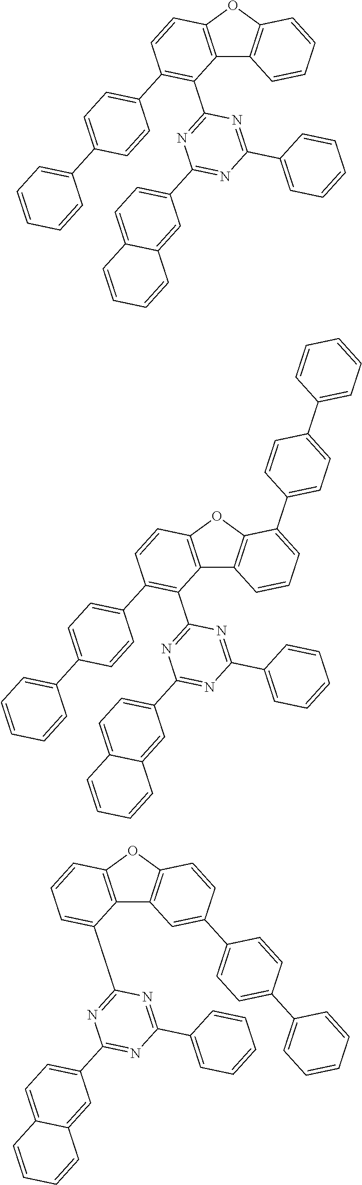

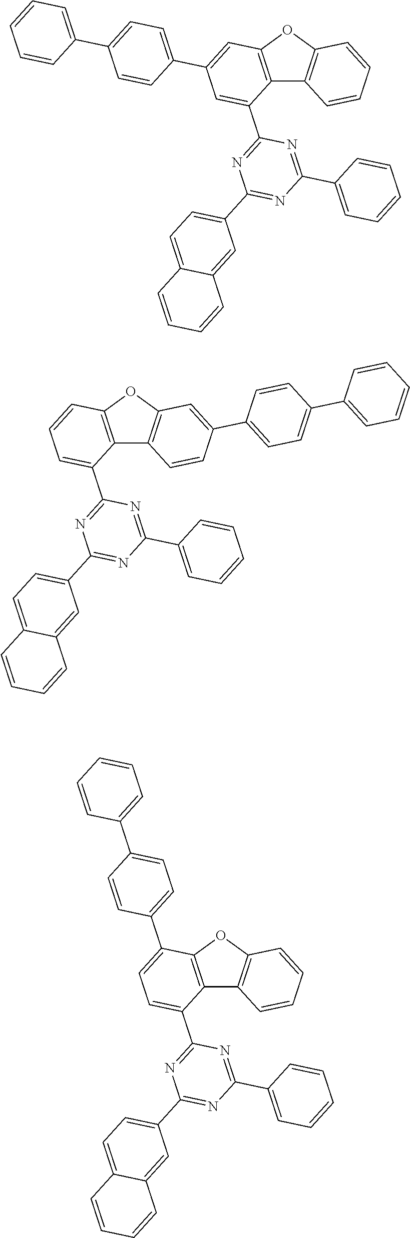 [SMILES: c1ccc(-c2ccc(-c3cc(-c4nc(-c5ccccc5)nc(-c5ccc6ccccc6c5)n4)c4c(c3)oc3ccccc34)cc2)cc1 ; c1ccc(-c2ccc(-c3ccc4c(c3)oc3cccc(-c5nc(-c6ccccc6)nc(-c6ccc7ccccc7c6)n5)c34)cc2)cc1 ; c1ccc(-c2ccc(-c3ccc(-c4nc(-c5ccccc5)nc(-c5ccc6ccccc6c5)n4)c4c3oc3ccccc34)cc2)cc1]

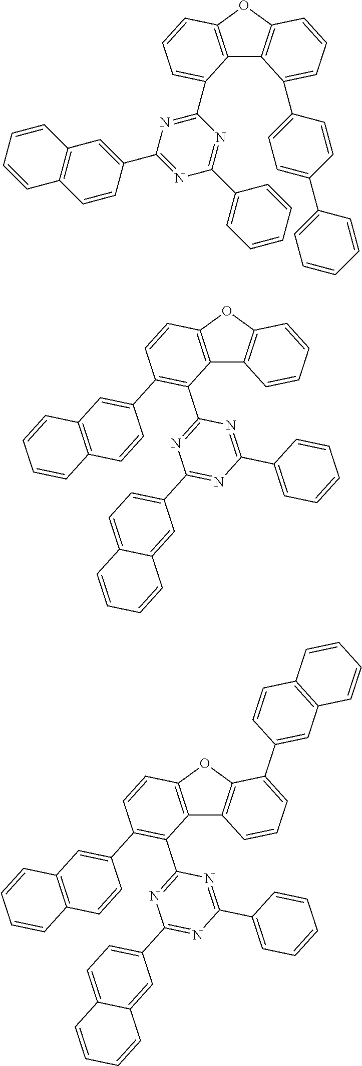 [SMILES: c1ccc(-c2ccc(-c3cccc4oc5cccc(-c6nc(-c7ccccc7)nc(-c7ccc8ccccc8c7)n6)c5c34)cc2)cc1 ; c1ccc(-c2nc(-c3ccc4ccccc4c3)nc(-c3c(-c4ccc5ccccc5c4)ccc4oc5ccccc5c34)n2)cc1 ; c1ccc(-c2nc(-c3ccc4ccccc4c3)nc(-c3c(-c4ccc5ccccc5c4)ccc4oc5c(-c6ccc7ccccc7c6)cccc5c34)n2)cc1]

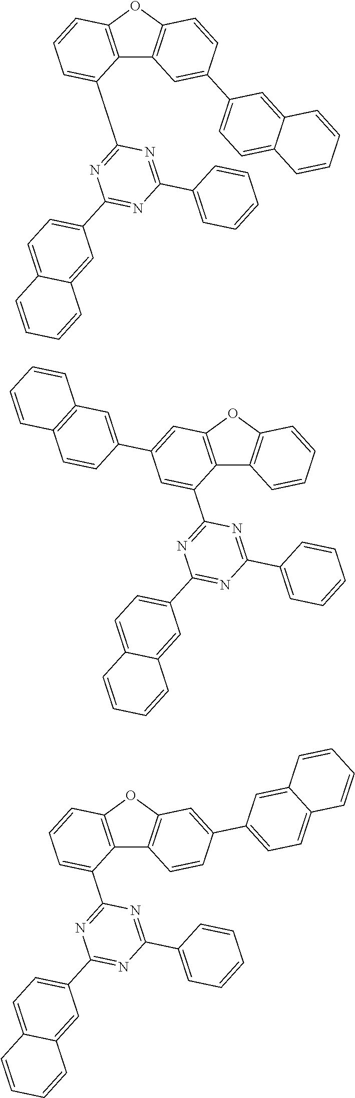

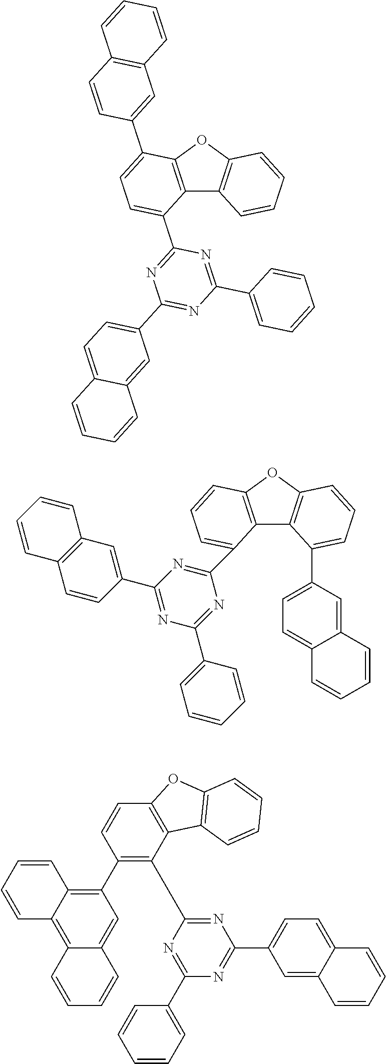 [SMILES: c1ccc(-c2nc(-c3ccc4ccccc4c3)nc(-c3ccc(-c4ccc5ccccc5c4)c4oc5ccccc5c34)n2)cc1 ; c1ccc(-c2nc(-c3ccc4ccccc4c3)nc(-c3cccc4oc5cccc(-c6ccc7ccccc7c6)c5c34)n2)cc1 ; c1ccc(-c2nc(Cc3c(-c4cc5ccccc5c5ccccc45)ccc4oc5ccccc5c34)nc(-c3ccc4ccccc4c3)n2)cc1]

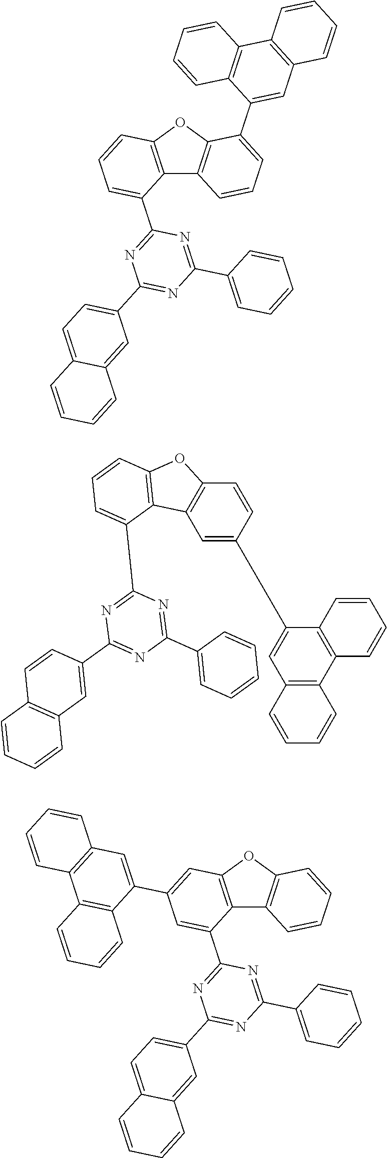

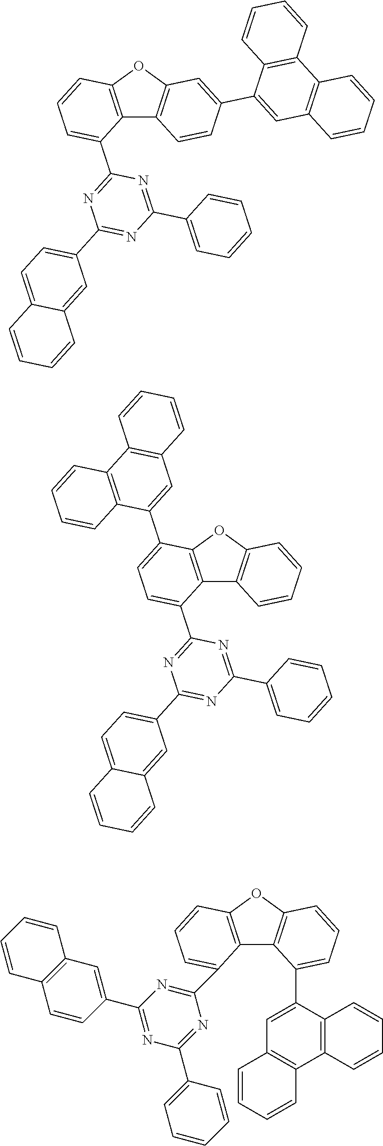 [SMILES: c1ccc(-c2nc(-c3ccc4ccccc4c3)nc(-c3cccc4oc5cc(-c6cc7ccccc7c7ccccc67)ccc5c34)n2)cc1 ; c1ccc(-c2nc(-c3ccc4ccccc4c3)nc(-c3ccc(-c4cc5ccccc5c5ccccc45)c4oc5ccccc5c34)n2)cc1 ; c1ccc(-c2nc(-c3ccc4ccccc4c3)nc(-c3cccc4oc5cccc(-c6cc7ccccc7c7ccccc67)c5c34)n2)cc1]

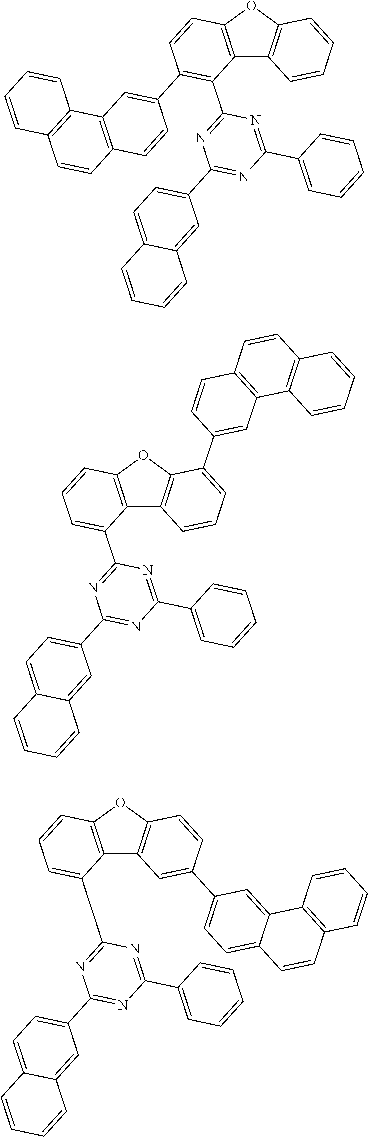 [SMILES: c1ccc(-c2nc(-c3ccc4ccccc4c3)nc(-c3c(-c4ccc5ccc6ccccc6c5c4)ccc4oc5ccccc5c34)n2)cc1 ; c1ccc(-c2nc(-c3ccc4ccccc4c3)nc(-c3cccc4oc5c(-c6ccc7ccc8ccccc8c7c6)cccc5c34)n2)cc1 ; c1ccc(-c2nc(Cc3cccc4oc5ccc(-c6ccc7ccc8ccccc8c7c6)cc5c34)nc(-c3ccc4ccccc4c3)n2)cc1]

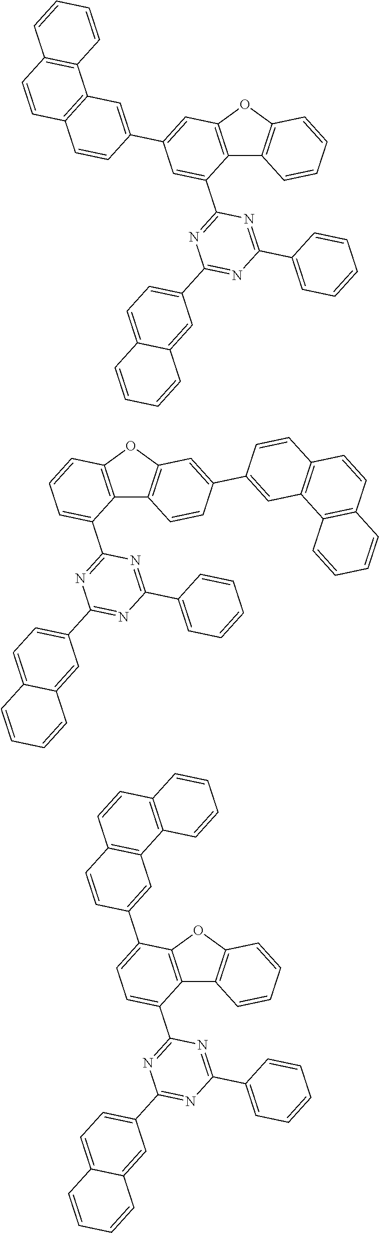 [SMILES: c1ccc(-c2nc(-c3ccc4ccccc4c3)nc(-c3cc(-c4ccc5ccc6ccccc6c5c4)cc4oc5ccccc5c34)n2)cc1 ; c1ccc(-c2nc(-c3ccc4ccccc4c3)nc(-c3cccc4oc5cc(-c6ccc7ccc8ccccc8c7c6)ccc5c34)n2)cc1 ; c1ccc(-c2nc(-c3ccc4ccccc4c3)nc(-c3ccc(-c4ccc5ccc6ccccc6c5c4)c4oc5ccccc5c34)n2)cc1]

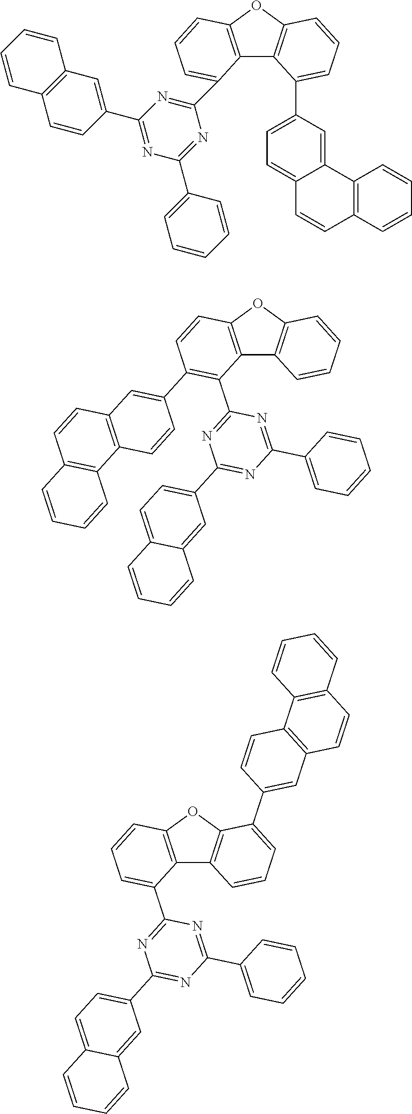 [SMILES: c1ccc(-c2nc(-c3ccc4ccccc4c3)nc(-c3cccc4oc5cccc(-c6ccc7ccc8ccccc8c7c6)c5c34)n2)cc1 ; c1ccc(-c2nc(-c3ccc4ccccc4c3)nc(-c3c(-c4ccc5c(ccc6ccccc65)c4)ccc4oc5ccccc5c34)n2)cc1 ; c1ccc(-c2nc(-c3ccc4ccccc4c3)nc(-c3cccc4oc5c(-c6ccc7c(ccc8ccccc87)c6)cccc5c34)n2)cc1]

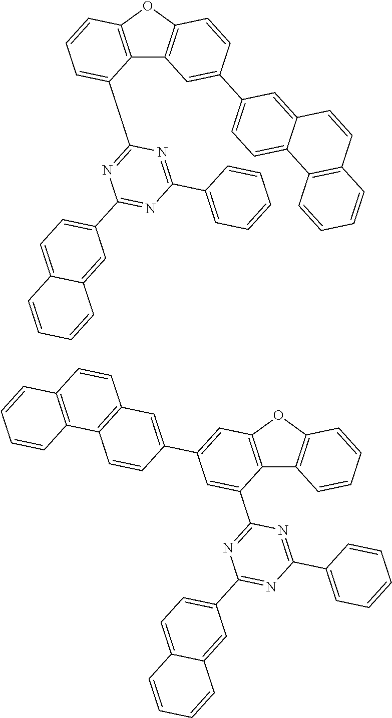

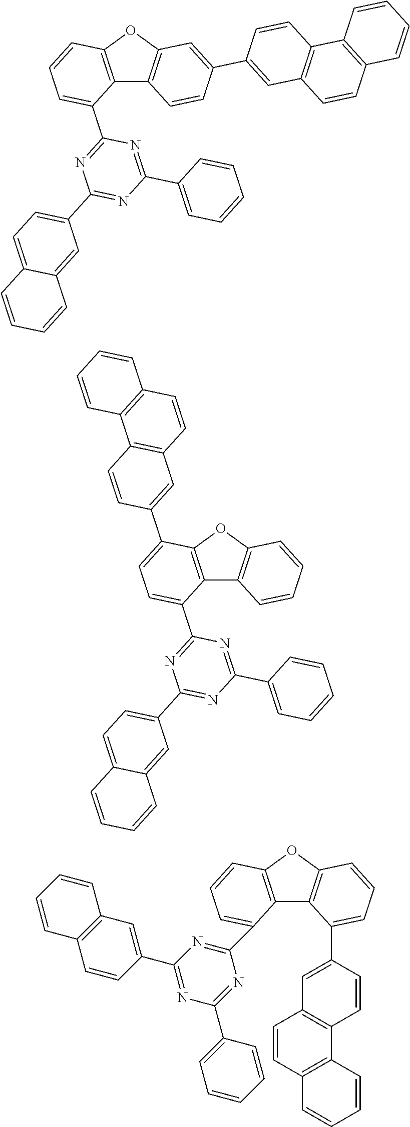 [SMILES: c1ccc(-c2nc(-c3ccc4ccccc4c3)nc(-c3cccc4oc5cc(-c6ccc7c(ccc8ccccc87)c6)ccc5c34)n2)cc1 ; c1ccc(-c2nc(-c3ccc4ccccc4c3)nc(-c3ccc(-c4ccc5c(ccc6ccccc65)c4)c4oc5ccccc5c34)n2)cc1 ; c1ccc(-c2nc(-c3ccc4ccccc4c3)nc(-c3cccc4oc5cccc(-c6ccc7c(ccc8ccccc87)c6)c5c34)n2)cc1]

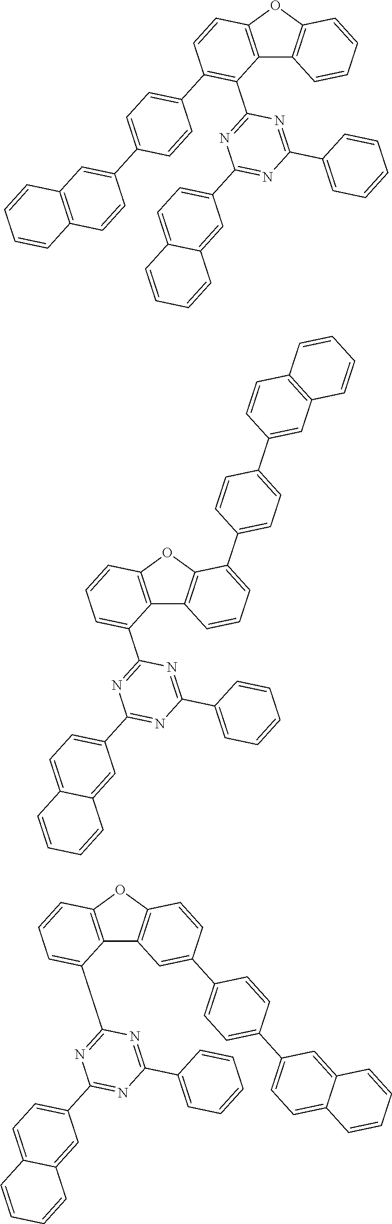 [SMILES: c1ccc(-c2nc(-c3ccc4ccccc4c3)nc(-c3c(-c4ccc(-c5ccc6ccccc6c5)cc4)ccc4oc5ccccc5c34)n2)cc1 ; c1ccc(-c2nc(-c3ccc4ccccc4c3)nc(-c3cccc4oc5c(-c6ccc(-c7ccc8ccccc8c7)cc6)cccc5c34)n2)cc1 ; c1ccc(-c2nc(Cc3cccc4oc5ccc(-c6ccc(-c7ccc8ccccc8c7)cc6)cc5c34)nc(-c3ccc4ccccc4c3)n2)cc1]

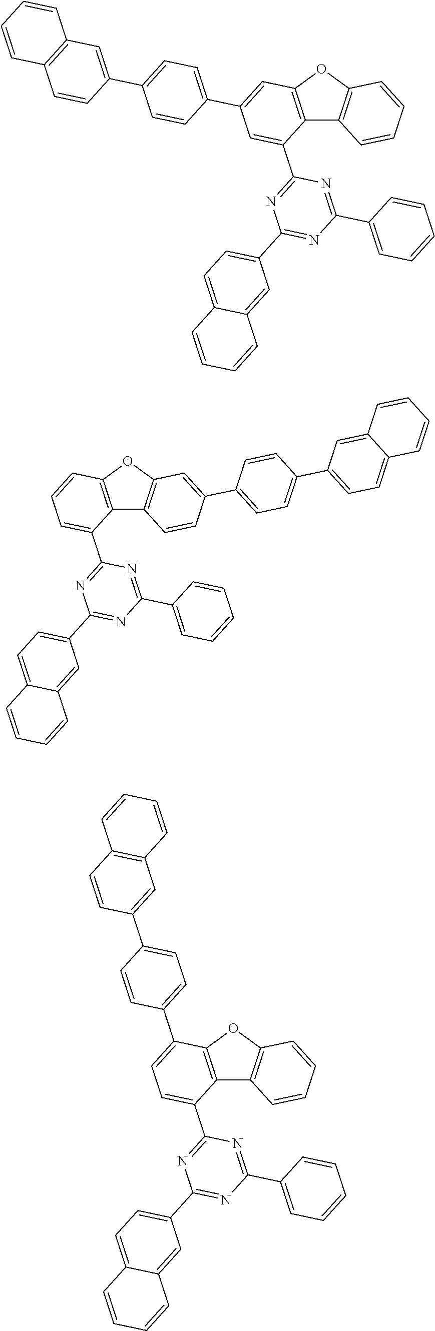 [SMILES: c1ccc(-c2nc(-c3ccc4ccccc4c3)nc(-c3cc(-c4ccc(-c5ccc6ccccc6c5)cc4)cc4oc5ccccc5c34)n2)cc1 ; c1ccc(-c2nc(-c3ccc4ccccc4c3)nc(-c3cccc4oc5cc(-c6ccc(-c7ccc8ccccc8c7)cc6)ccc5c34)n2)cc1 ; c1ccc(-c2nc(-c3ccc4ccccc4c3)nc(-c3ccc(-c4ccc(-c5ccc6ccccc6c5)cc4)c4oc5ccccc5c34)n2)cc1]

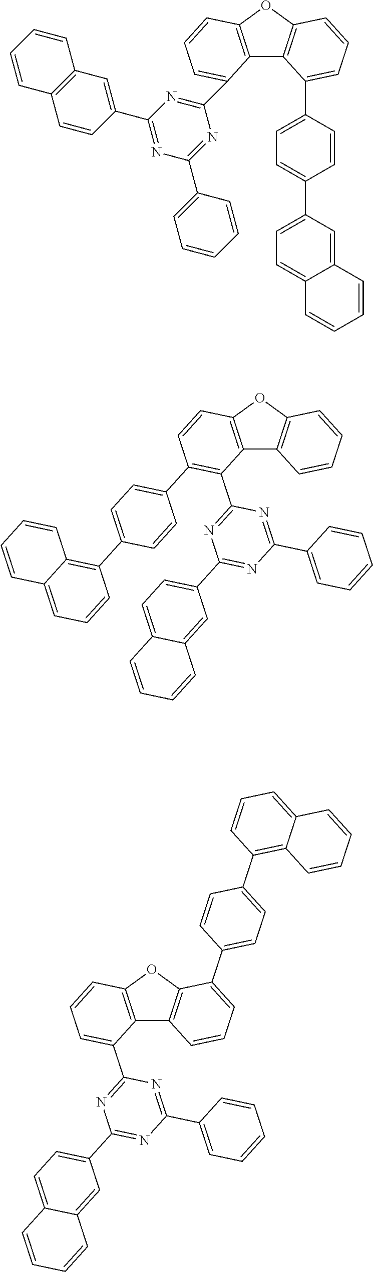 [SMILES: c1ccc(-c2nc(-c3ccc4ccccc4c3)nc(-c3cccc4oc5cccc(-c6ccc(-c7ccc8ccccc8c7)cc6)c5c34)n2)cc1 ; c1ccc(-c2nc(-c3ccc4ccccc4c3)nc(-c3c(-c4ccc(-c5cccc6ccccc56)cc4)ccc4oc5ccccc5c34)n2)cc1 ; c1ccc(-c2nc(-c3ccc4ccccc4c3)nc(-c3cccc4oc5c(-c6ccc(-c7cccc8ccccc78)cc6)cccc5c34)n2)cc1]

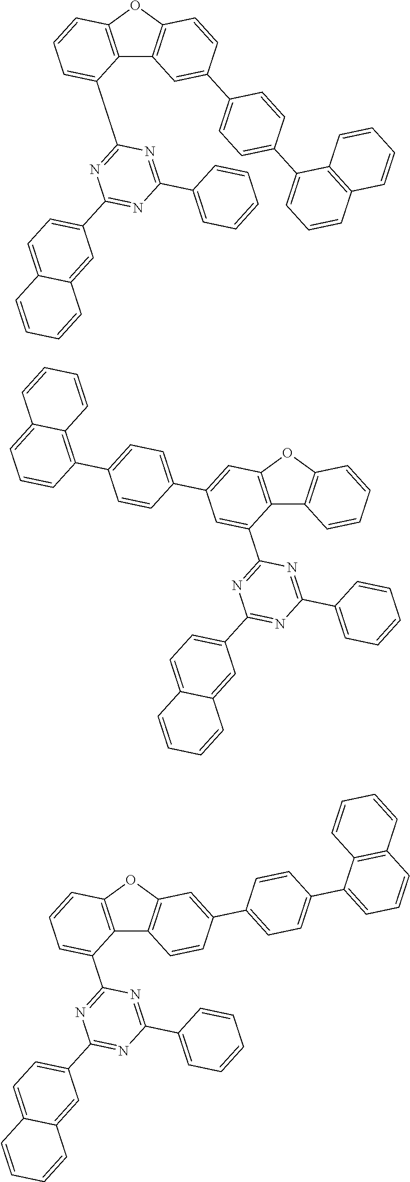

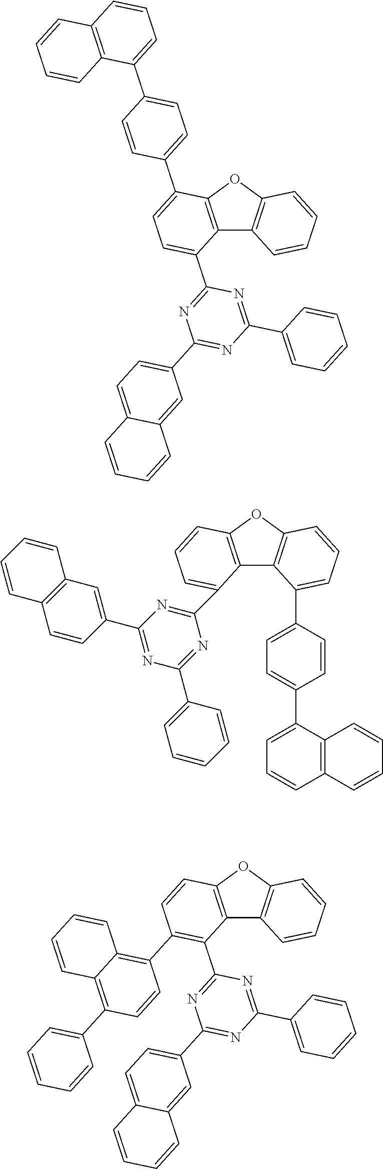 [SMILES: c1ccc(-c2nc(-c3ccc4ccccc4c3)nc(-c3ccc(-c4ccc(-c5cccc6ccccc56)cc4)c4oc5ccccc5c34)n2)cc1 ; c1ccc(-c2nc(-c3ccc4ccccc4c3)nc(-c3cccc4oc5cccc(-c6ccc(-c7cccc8ccccc78)cc6)c5c34)n2)cc1 ; c1ccc(-c2nc(-c3ccc4ccccc4c3)nc(-c3c(-c4ccc(-c5ccccc5)c5ccccc45)ccc4oc5ccccc5c34)n2)cc1]

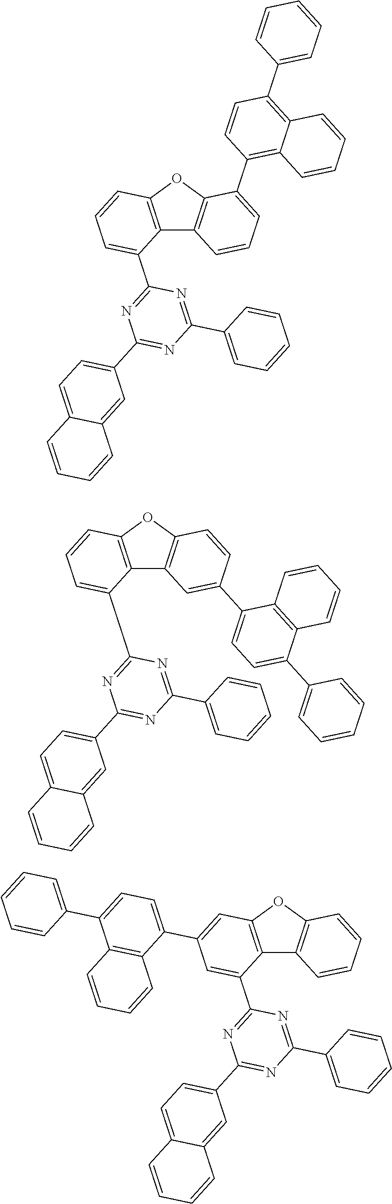

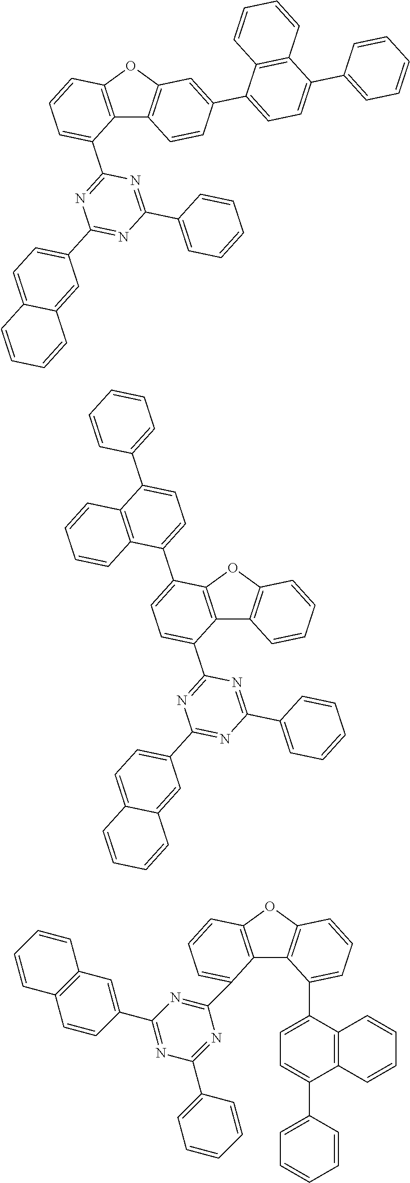 [SMILES: c1ccc(-c2nc(-c3ccc4ccccc4c3)nc(-c3cccc4oc5cc(-c6ccc(-c7ccccc7)c7ccccc67)ccc5c34)n2)cc1 ; c1ccc(-c2nc(-c3ccc4ccccc4c3)nc(-c3ccc(-c4ccc(-c5ccccc5)c5ccccc45)c4oc5ccccc5c34)n2)cc1 ; c1ccc(-c2nc(-c3ccc4ccccc4c3)nc(-c3cccc4oc5cccc(-c6ccc(-c7ccccc7)c7ccccc67)c5c34)n2)cc1]

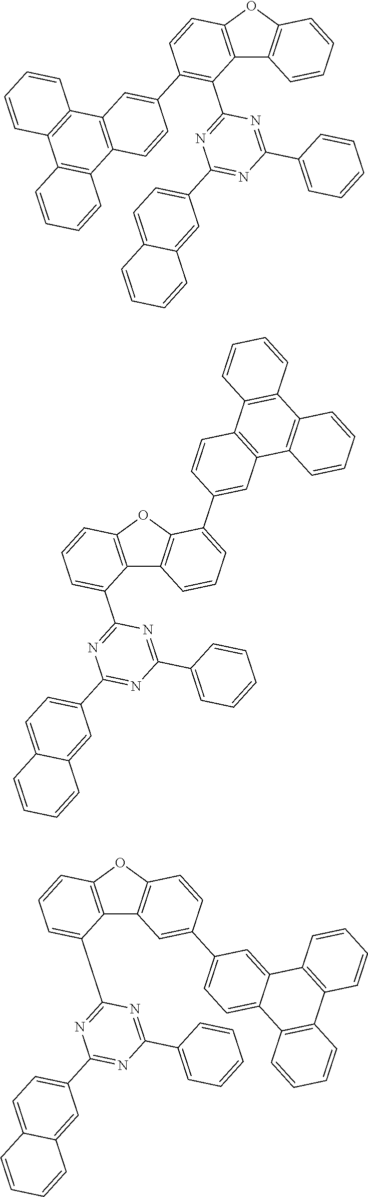 [SMILES: c1ccc(-c2nc(-c3ccc4ccccc4c3)nc(-c3c(-c4ccc5c6ccccc6c6ccccc6c5c4)ccc4oc5ccccc5c34)n2)cc1 ; c1ccc(-c2nc(-c3ccc4ccccc4c3)nc(-c3cccc4oc5c(-c6ccc7c8ccccc8c8ccccc8c7c6)cccc5c34)n2)cc1 ; c1ccc(-c2nc(Cc3cccc4oc5ccc(-c6ccc7c8ccccc8c8ccccc8c7c6)cc5c34)nc(-c3ccc4ccccc4c3)n2)cc1]

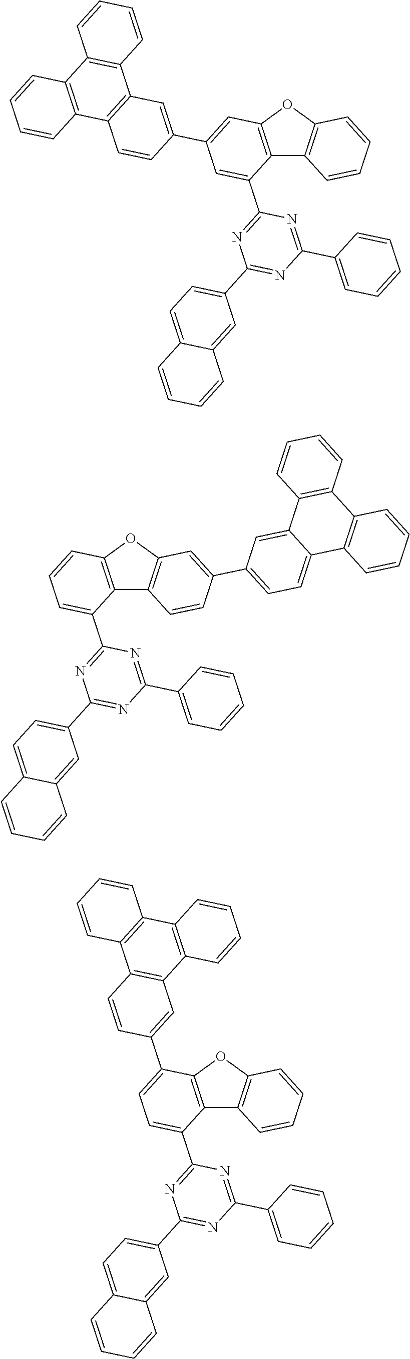 [SMILES: c1ccc(-c2nc(-c3ccc4ccccc4c3)nc(-c3cc(-c4ccc5c6ccccc6c6ccccc6c5c4)cc4oc5ccccc5c34)n2)cc1 ; c1ccc(-c2nc(-c3ccc4ccccc4c3)nc(-c3cccc4oc5cc(-c6ccc7c8ccccc8c8ccccc8c7c6)ccc5c34)n2)cc1 ; c1ccc(-c2nc(-c3ccc4ccccc4c3)nc(-c3ccc(-c4ccc5c6ccccc6c6ccccc6c5c4)c4oc5ccccc5c34)n2)cc1]

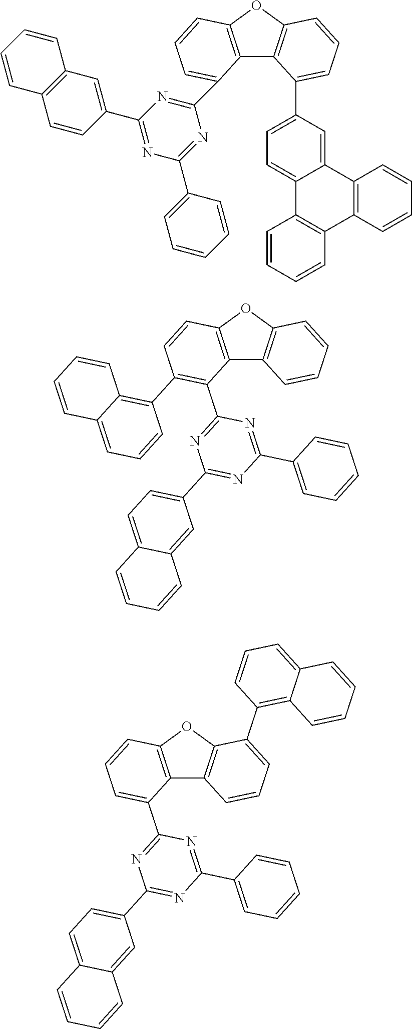

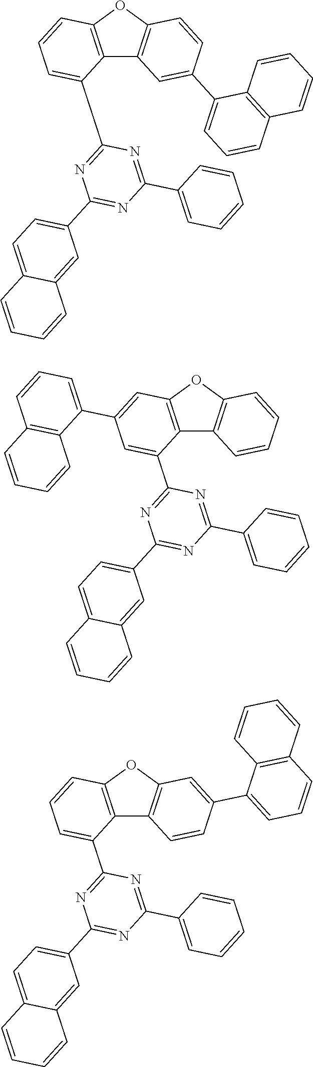

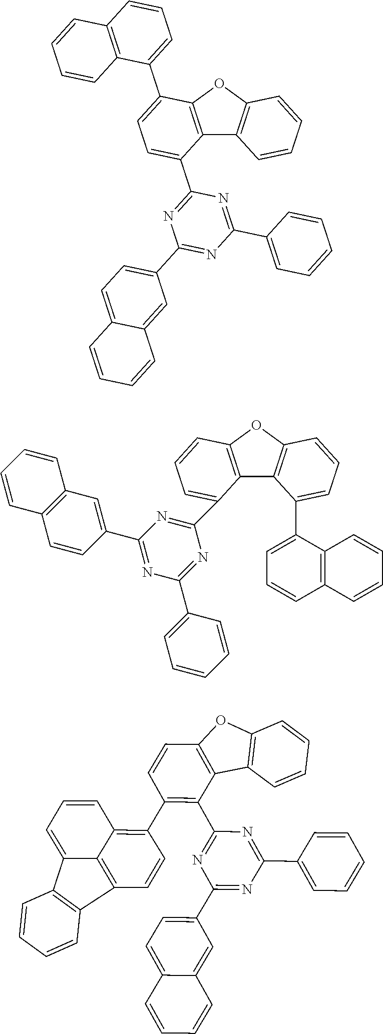

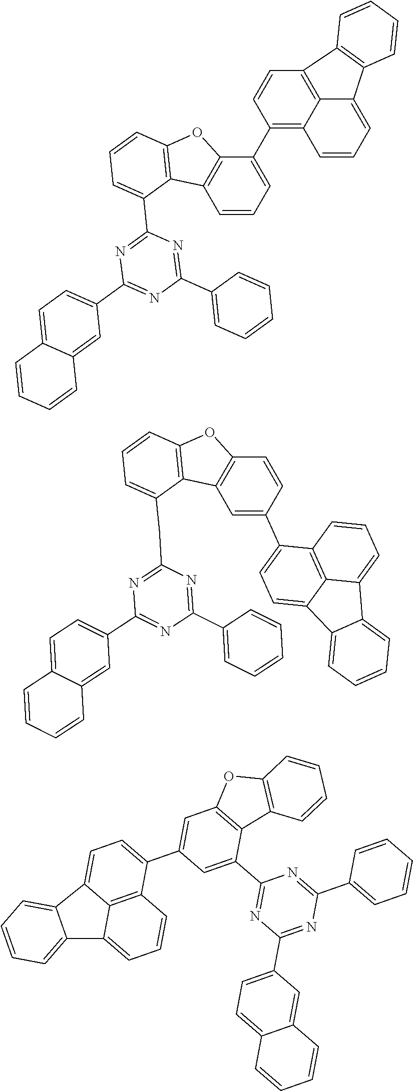 [SMILES: c1ccc(-c2nc(-c3ccc4ccccc4c3)nc(-c3cccc4oc5c(-c6ccc7c8c(cccc68)-c6ccccc6-7)cccc5c34)n2)cc1 ; c1ccc(-c2nc(Cc3cccc4oc5ccc(-c6ccc7c8c(cccc68)-c6ccccc6-7)cc5c34)nc(-c3ccc4ccccc4c3)n2)cc1 ; c1ccc(-c2nc(-c3ccc4ccccc4c3)nc(-c3cc(-c4ccc5c6c(cccc46)-c4ccccc4-5)cc4oc5ccccc5c34)n2)cc1]

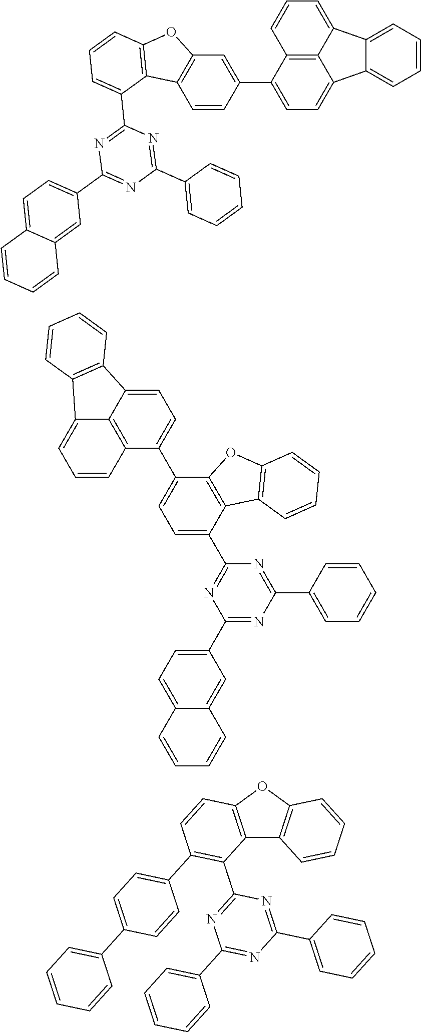

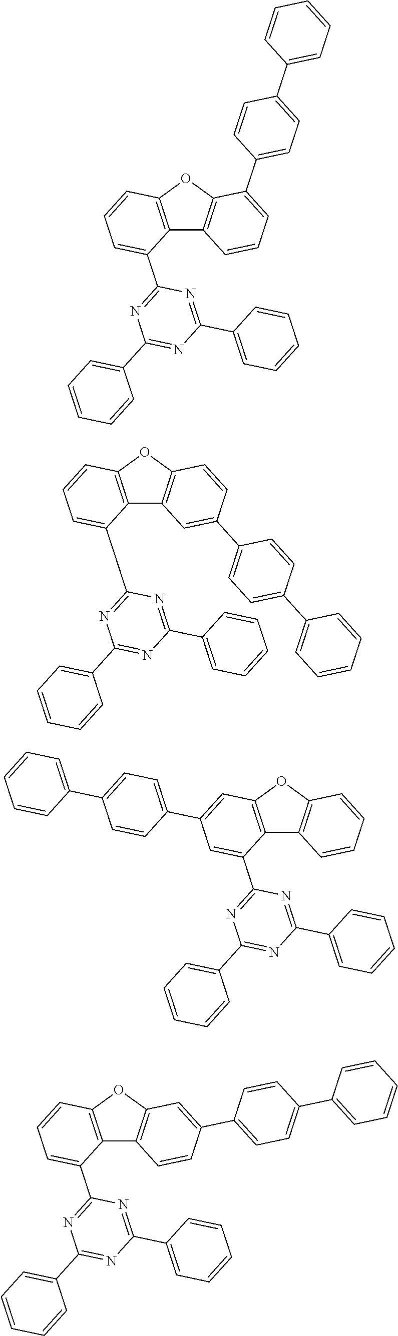

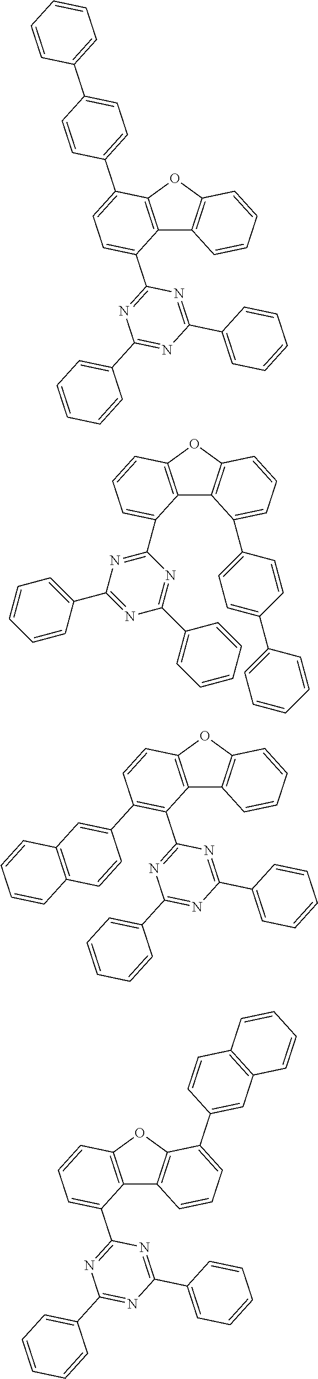

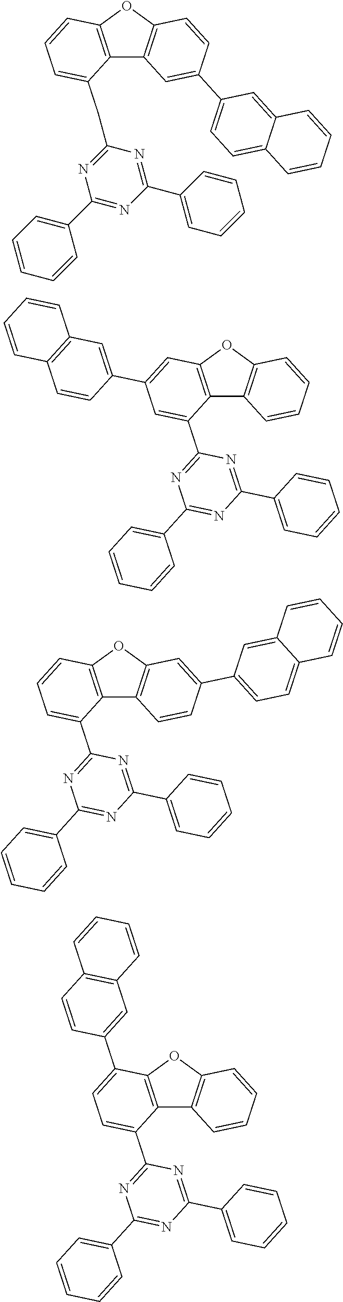

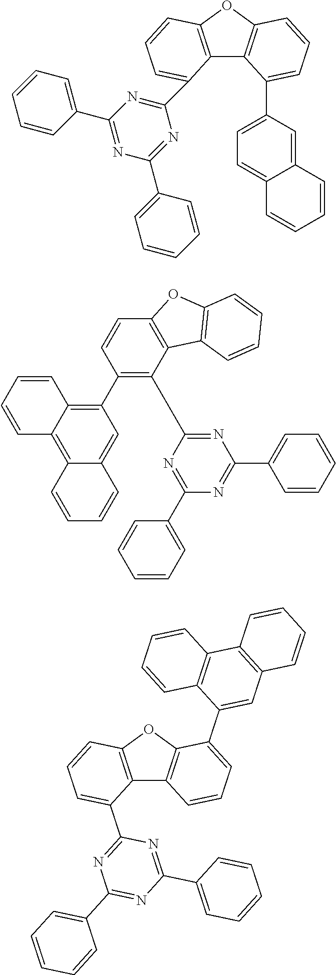

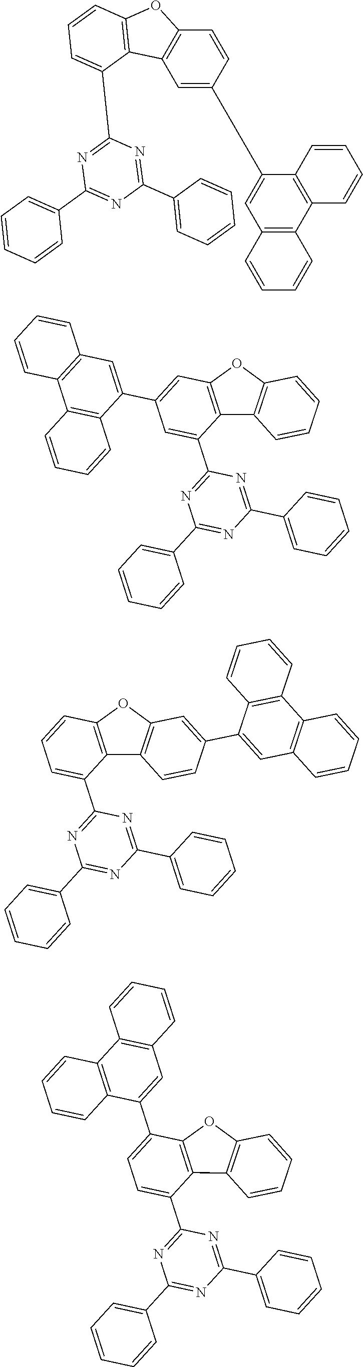 [SMILES: c1ccc(-c2nc(Cc3cccc4oc5ccc(CCc6cc7ccccc7c7ccccc67)cc5c34)nc(-c3ccccc3)n2)cc1 ; c1ccc(-c2nc(-c3ccccc3)nc(-c3cc(-c4cc5ccccc5c5ccccc45)cc4oc5ccccc5c34)n2)cc1 ; c1ccc(-c2nc(-c3ccccc3)nc(-c3cccc4oc5cc(-c6cc7ccccc7c7ccccc67)ccc5c34)n2)cc1 ; c1ccc(-c2nc(-c3ccccc3)nc(-c3ccc(-c4cc5ccccc5c5ccccc45)c4oc5ccccc5c34)n2)cc1]

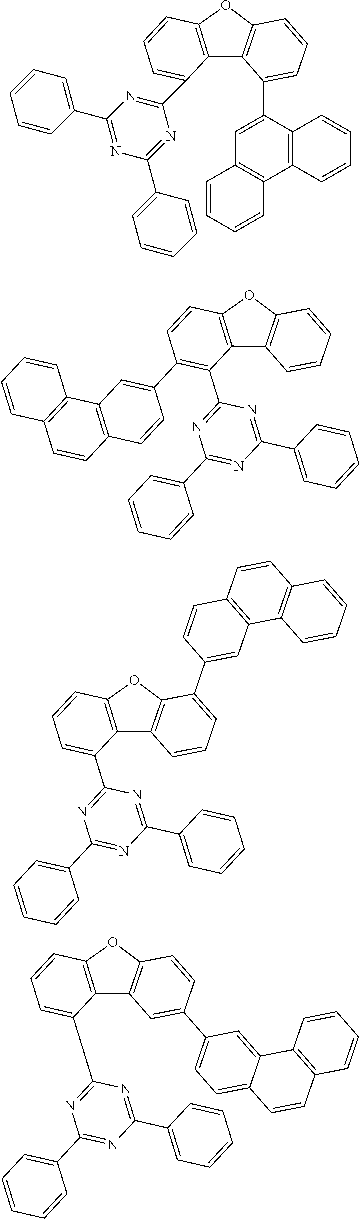

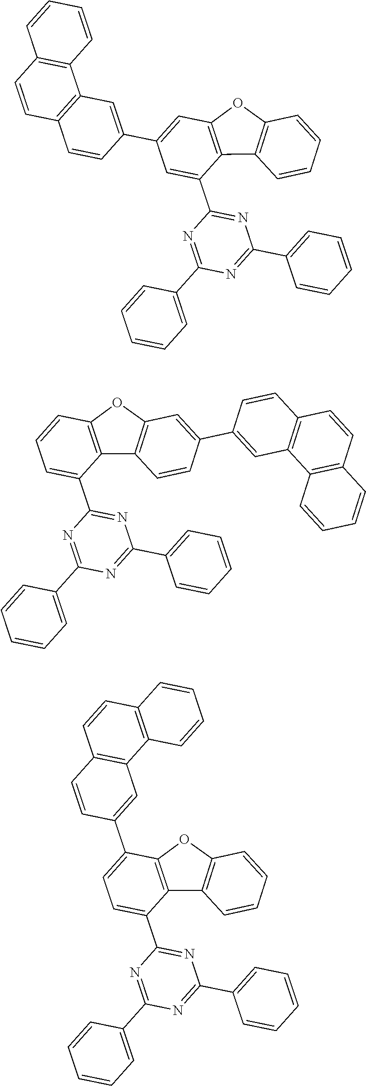 [SMILES: c1ccc(-c2nc(-c3ccccc3)nc(-c3cc(-c4ccc5ccc6ccccc6c5c4)cc4oc5ccccc5c34)n2)cc1 ; c1ccc(-c2nc(-c3ccccc3)nc(-c3cccc4oc5cc(-c6ccc7ccc8ccccc8c7c6)ccc5c34)n2)cc1 ; c1ccc(-c2nc(-c3ccccc3)nc(-c3ccc(-c4ccc5ccc6ccccc6c5c4)c4oc5ccccc5c34)n2)cc1]

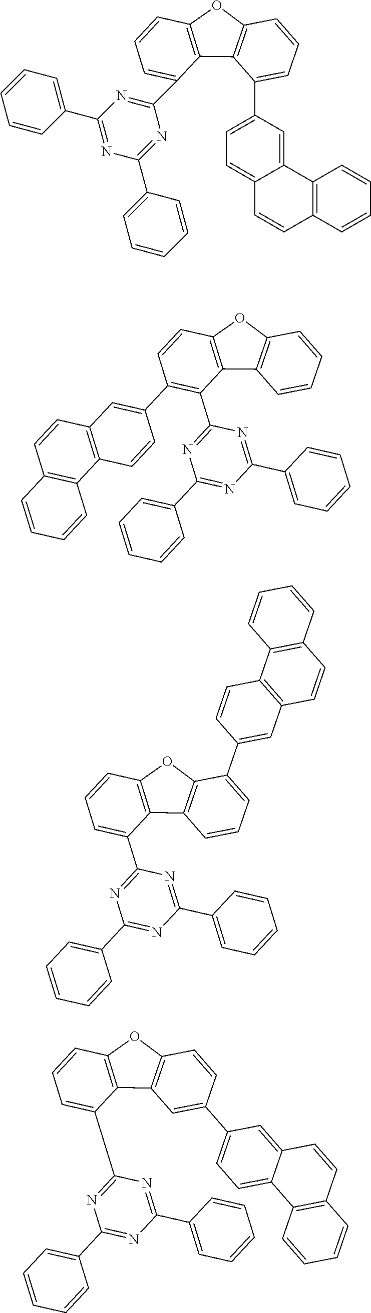

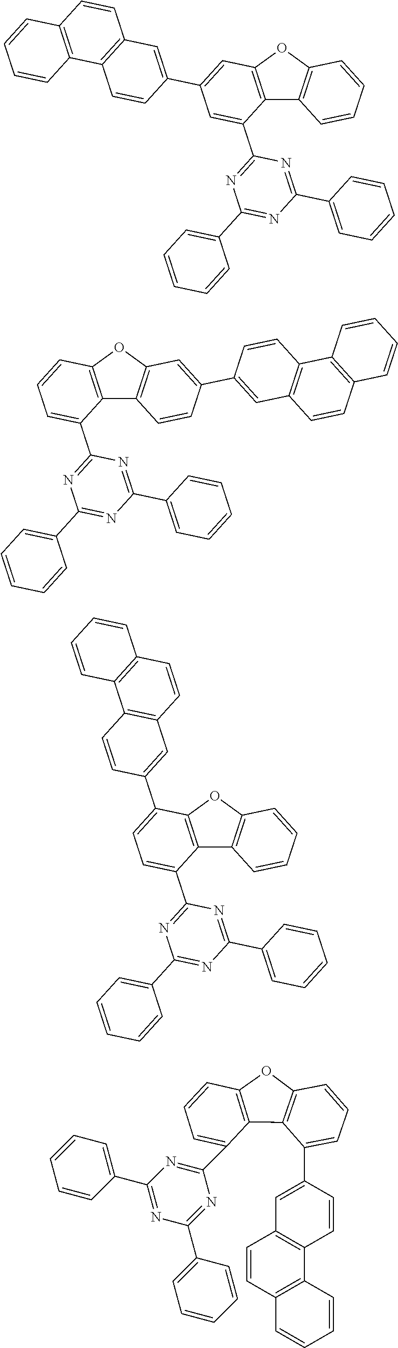 [SMILES: c1ccc(-c2nc(-c3ccccc3)nc(-c3cc(-c4ccc5c(ccc6ccccc65)c4)cc4oc5ccccc5c34)n2)cc1 ; c1ccc(-c2nc(-c3ccccc3)nc(-c3cccc4oc5cc(-c6ccc7c(ccc8ccccc87)c6)ccc5c34)n2)cc1 ; c1ccc(-c2nc(-c3ccccc3)nc(-c3ccc(-c4ccc5c(ccc6ccccc65)c4)c4oc5ccccc5c34)n2)cc1 ; c1ccc(-c2nc(-c3ccccc3)nc(-c3cccc4oc5cccc(-c6ccc7c(ccc8ccccc87)c6)c5c34)n2)cc1]

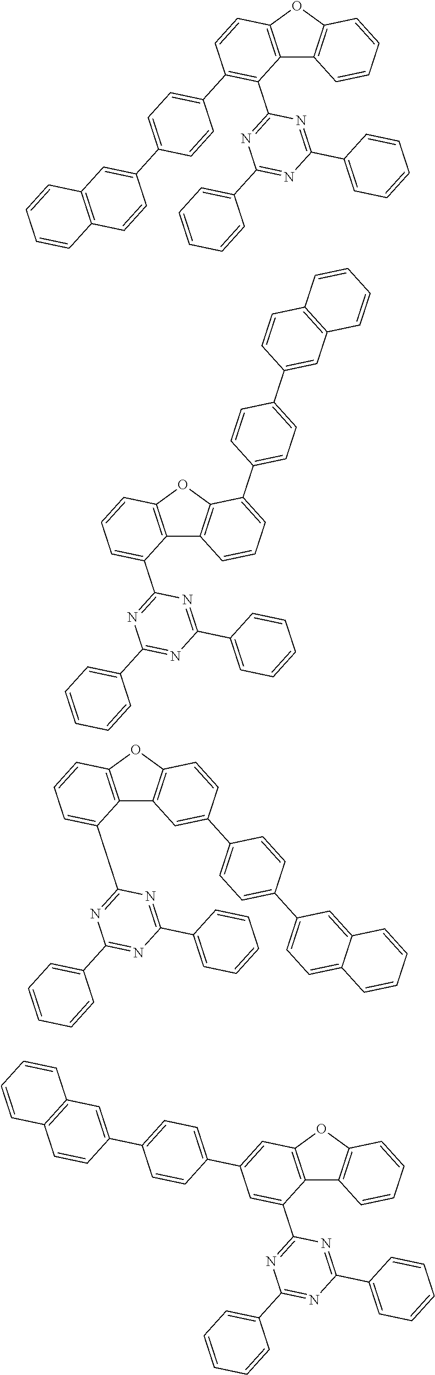

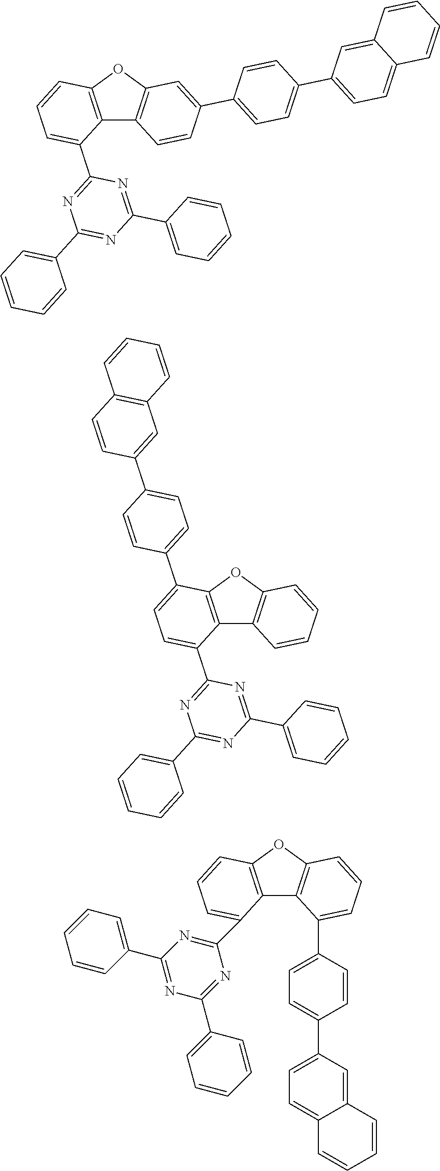 [SMILES: c1ccc(-c2nc(-c3ccccc3)nc(-c3cccc4oc5cc(-c6ccc(-c7ccc8ccccc8c7)cc6)ccc5c34)n2)cc1 ; c1ccc(-c2nc(-c3ccccc3)nc(-c3ccc(-c4ccc(-c5ccc6ccccc6c5)cc4)c4oc5ccccc5c34)n2)cc1 ; c1ccc(-c2nc(-c3ccccc3)nc(-c3cccc4oc5cccc(-c6ccc(-c7ccc8ccccc8c7)cc6)c5c34)n2)cc1]

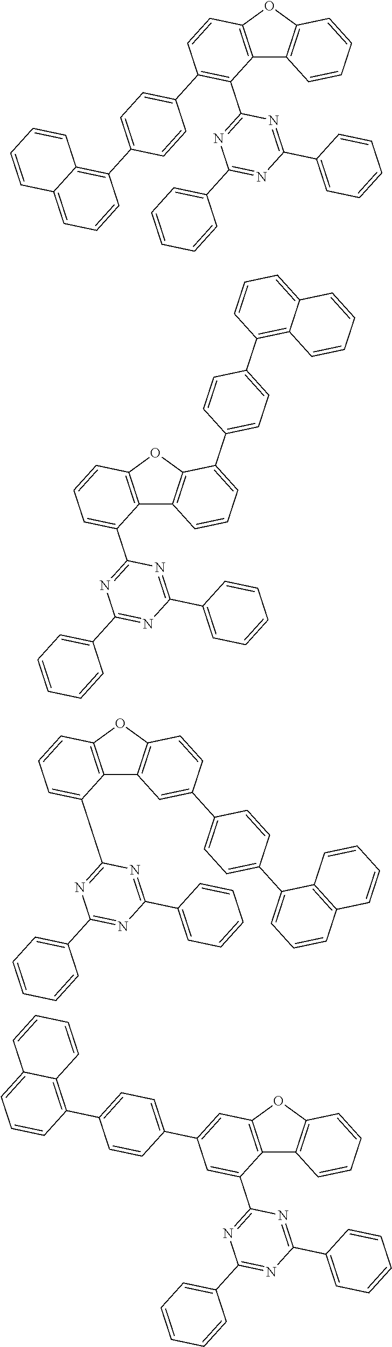

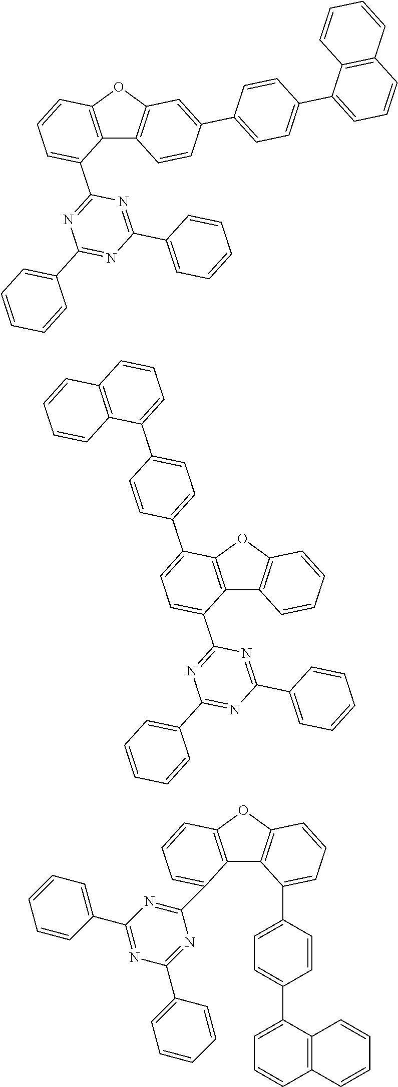

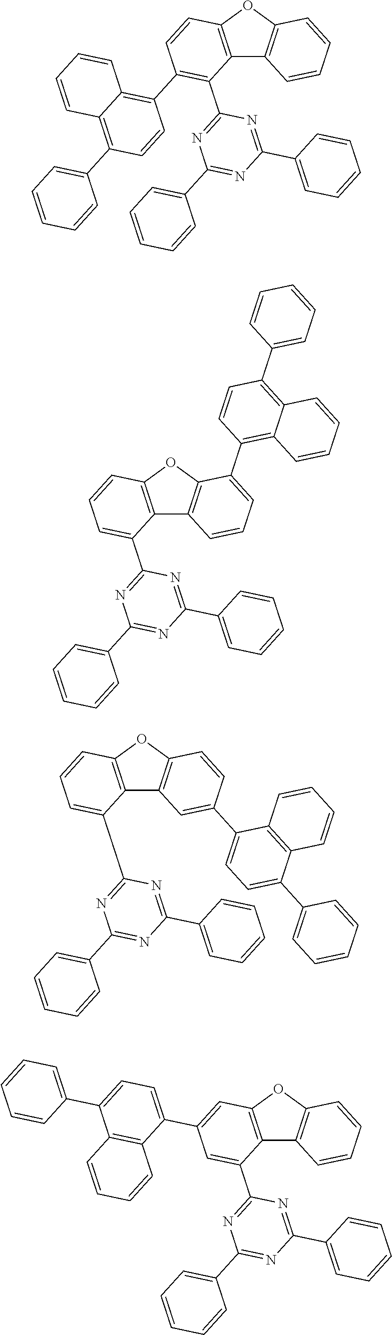

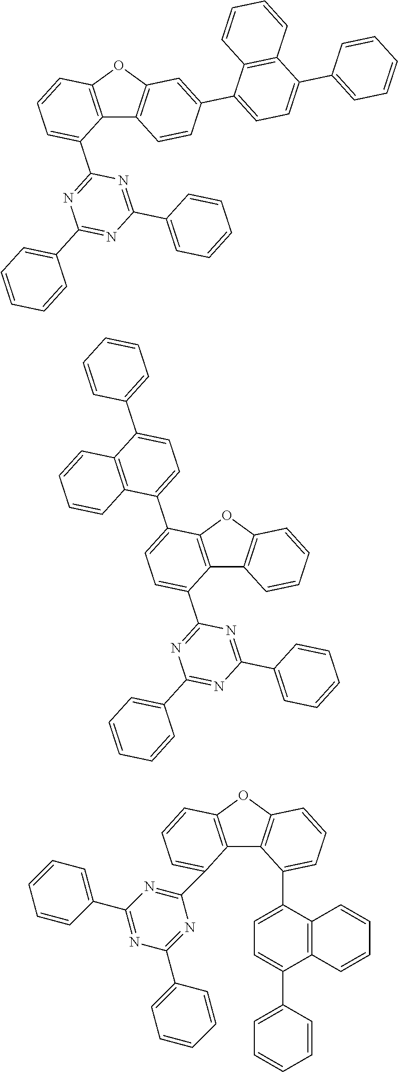 [SMILES: c1ccc(-c2nc(-c3ccccc3)nc(-c3cccc4oc5cc(-c6ccc(-c7ccccc7)c7ccccc67)ccc5c34)n2)cc1 ; c1ccc(-c2nc(-c3ccccc3)nc(-c3ccc(-c4ccc(-c5ccccc5)c5ccccc45)c4oc5ccccc5c34)n2)cc1 ; c1ccc(-c2nc(-c3ccccc3)nc(-c3cccc4oc5cccc(-c6ccc(-c7ccccc7)c7ccccc67)c5c34)n2)cc1]

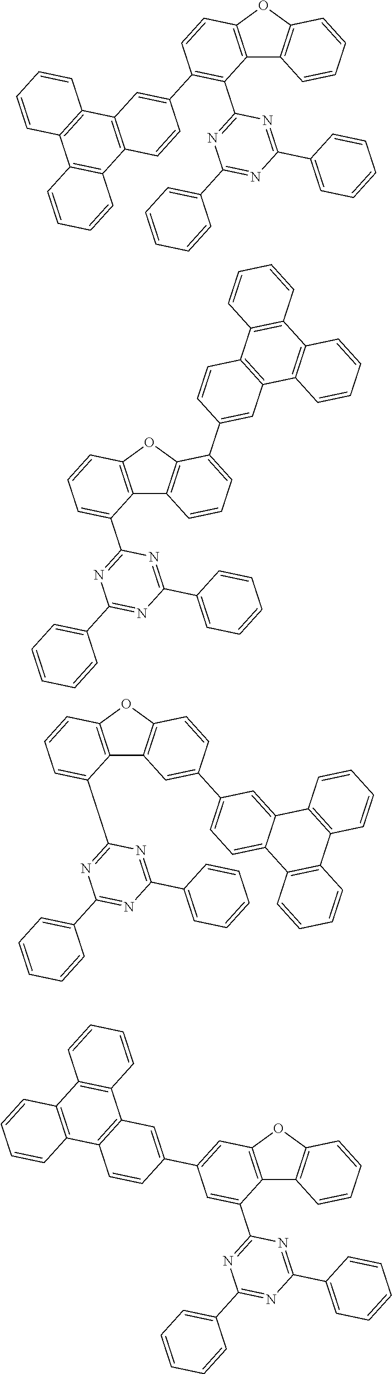

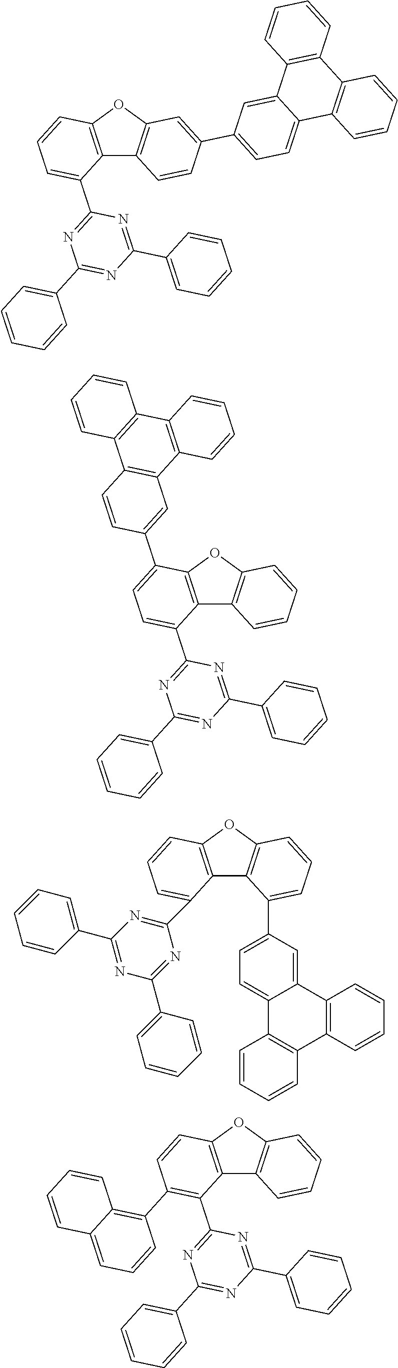

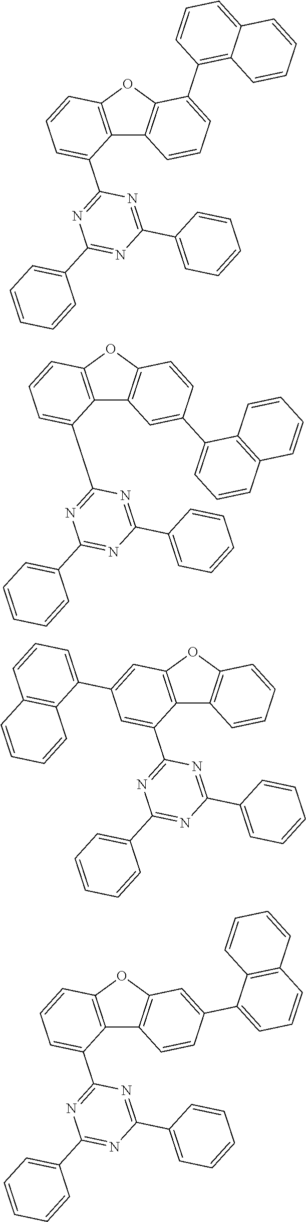

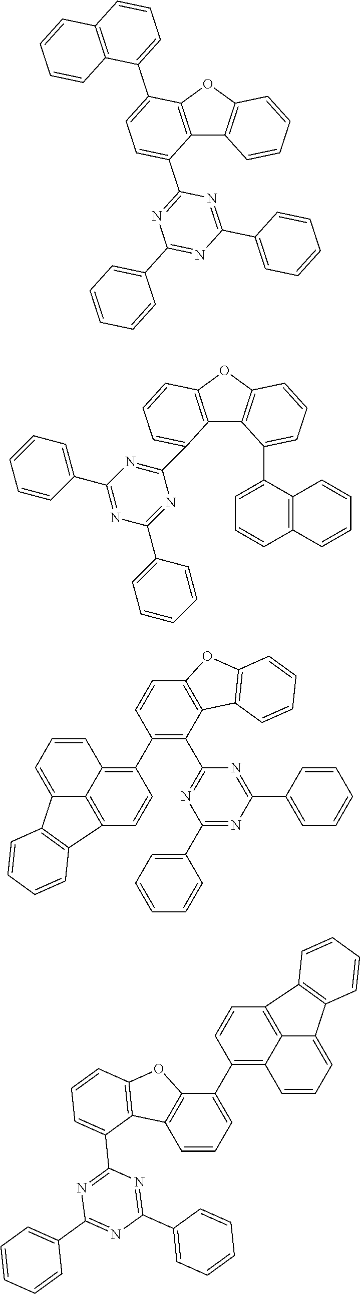 [SMILES: c1ccc(-c2nc(-c3ccccc3)nc(-c3ccc(-c4cccc5ccccc45)c4oc5ccccc5c34)n2)cc1 ; c1ccc(-c2nc(-c3ccccc3)nc(-c3cccc4oc5cccc(-c6cccc7ccccc67)c5c34)n2)cc1 ; c1ccc(-c2nc(-c3ccccc3)nc(-c3c(-c4ccc5c6c(cccc46)-c4ccccc4-5)ccc4oc5ccccc5c34)n2)cc1 ; c1ccc(-c2nc(-c3ccccc3)nc(-c3cccc4oc5c(-c6ccc7c8c(cccc68)-c6ccccc6-7)cccc5c34)n2)cc1]

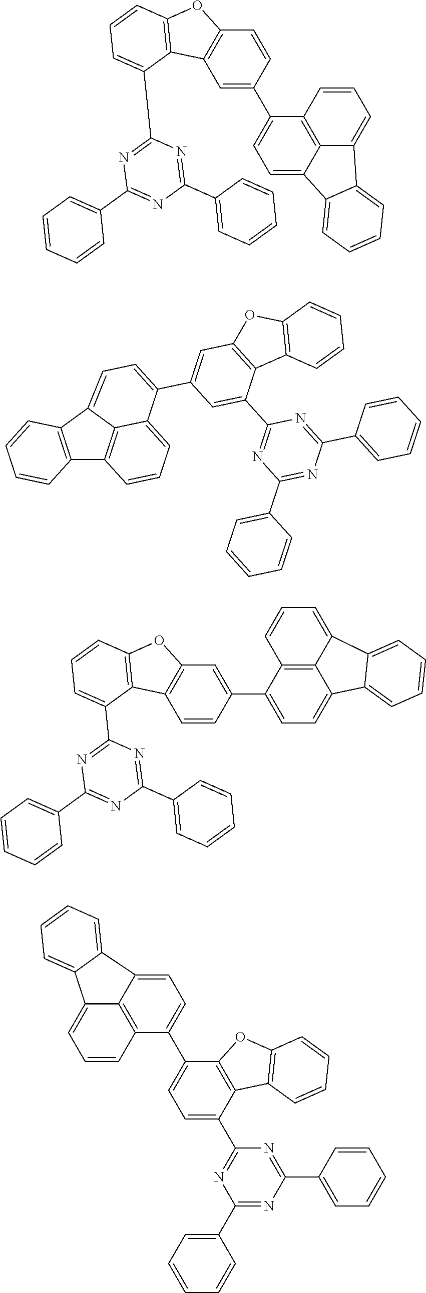

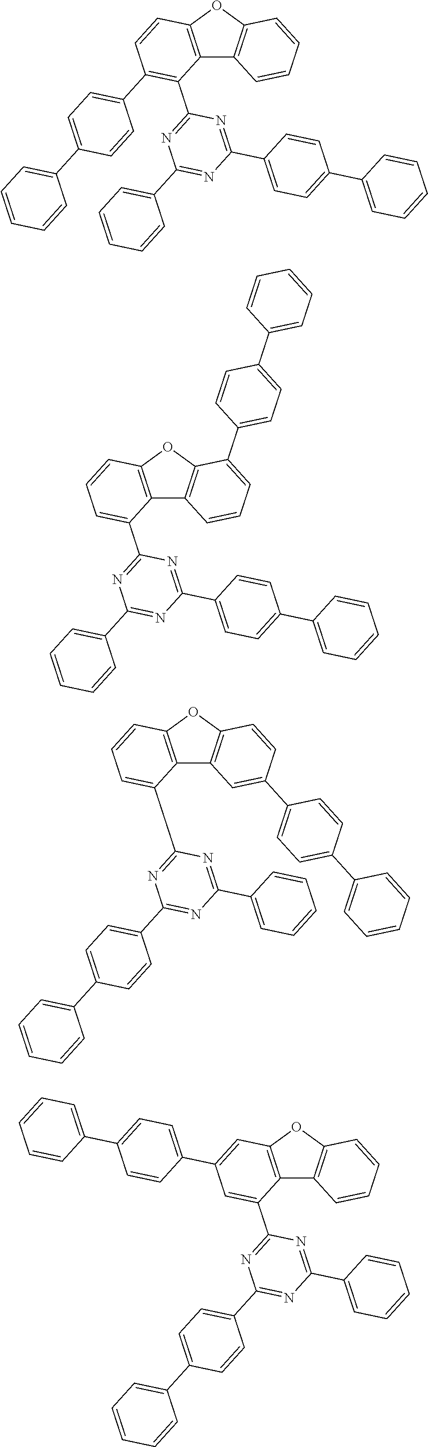

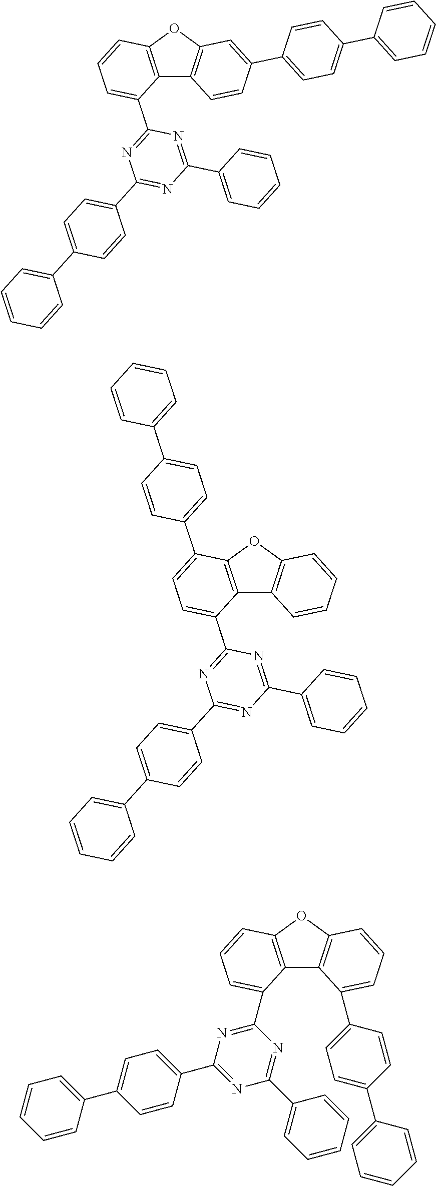 [SMILES: c1ccc(-c2ccc(-c3ccc4c(c3)oc3cccc(-c5nc(-c6ccccc6)nc(-c6ccc(-c7ccccc7)cc6)n5)c34)cc2)cc1 ; c1ccc(-c2ccc(-c3nc(-c4ccccc4)nc(-c4ccc(-c5ccc(-c6ccccc6)cc5)c5oc6ccccc6c45)n3)cc2)cc1 ; c1ccc(-c2ccc(-c3nc(-c4ccccc4)nc(-c4cccc5oc6cccc(-c7ccc(-c8ccccc8)cc7)c6c45)n3)cc2)cc1]

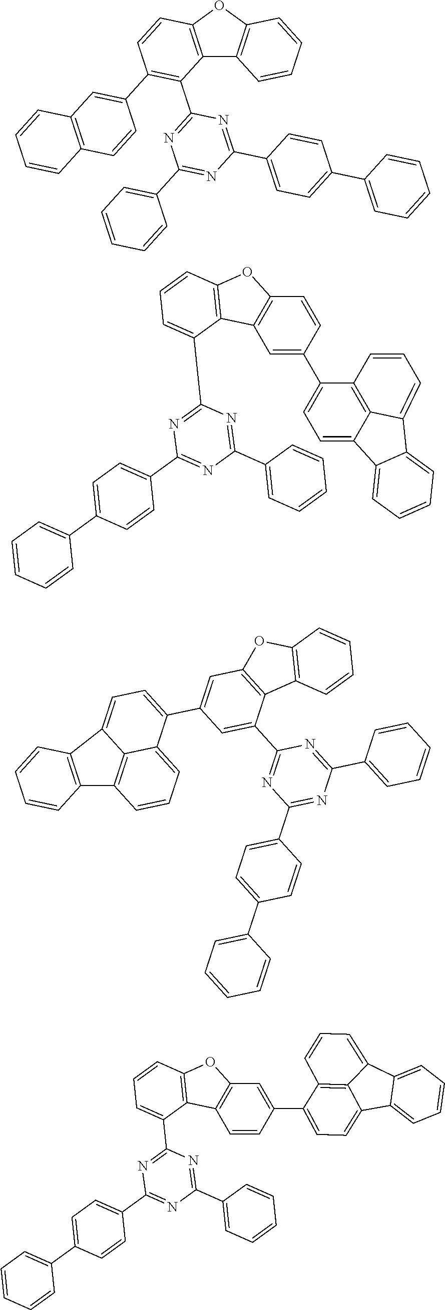

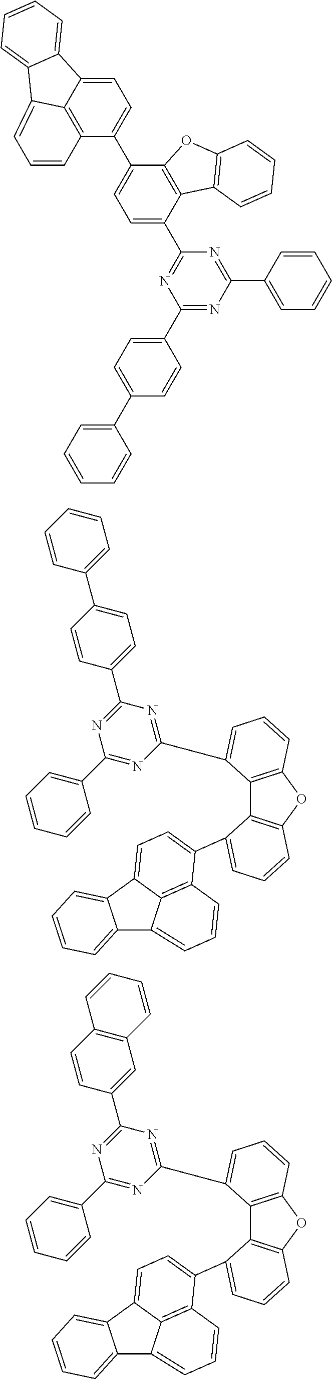

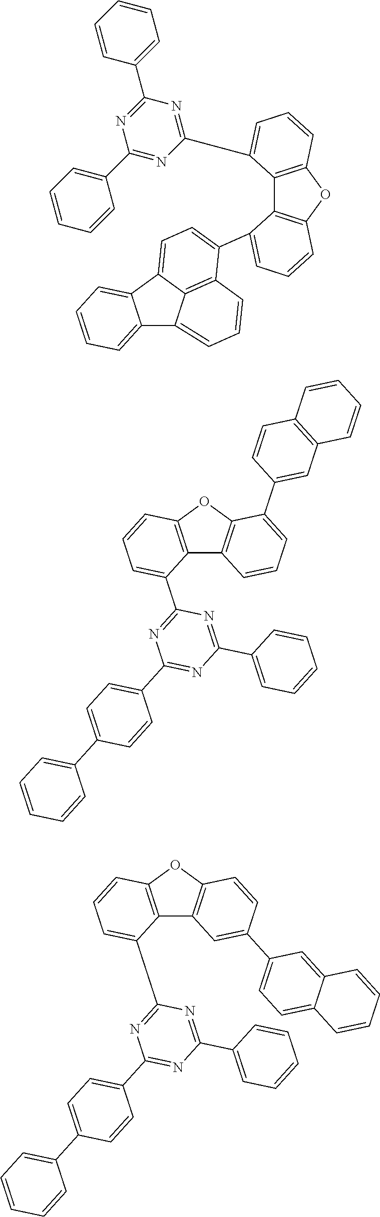 [SMILES: c1ccc(-c2nc(Cc3cccc4oc5cccc(-c6ccc7c8c(cccc68)-c6ccccc6-7)c5c34)nc(-c3ccccc3)n2)cc1 ; c1ccc(-c2ccc(-c3nc(-c4ccccc4)nc(-c4cccc5oc6c(-c7ccc8ccccc8c7)cccc6c45)n3)cc2)cc1 ; c1ccc(-c2ccc(-c3nc(Cc4cccc5oc6ccc(-c7ccc8ccccc8c7)cc6c45)nc(-c4ccccc4)n3)cc2)cc1]

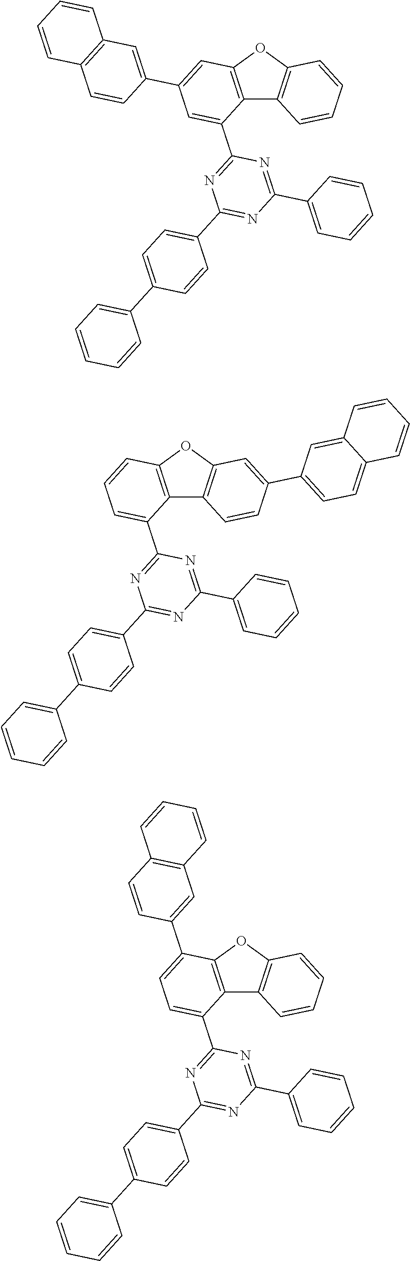 [SMILES: c1ccc(-c2ccc(-c3nc(-c4ccccc4)nc(-c4cc(-c5ccc6ccccc6c5)cc5oc6ccccc6c45)n3)cc2)cc1 ; c1ccc(-c2ccc(-c3nc(-c4ccccc4)nc(-c4cccc5oc6cc(-c7ccc8ccccc8c7)ccc6c45)n3)cc2)cc1 ; c1ccc(-c2ccc(-c3nc(-c4ccccc4)nc(-c4ccc(-c5ccc6ccccc6c5)c5oc6ccccc6c45)n3)cc2)cc1]

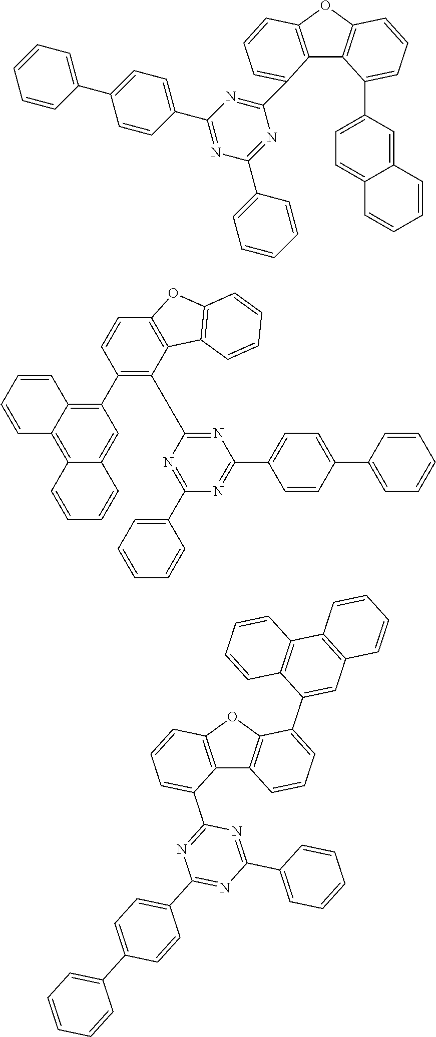

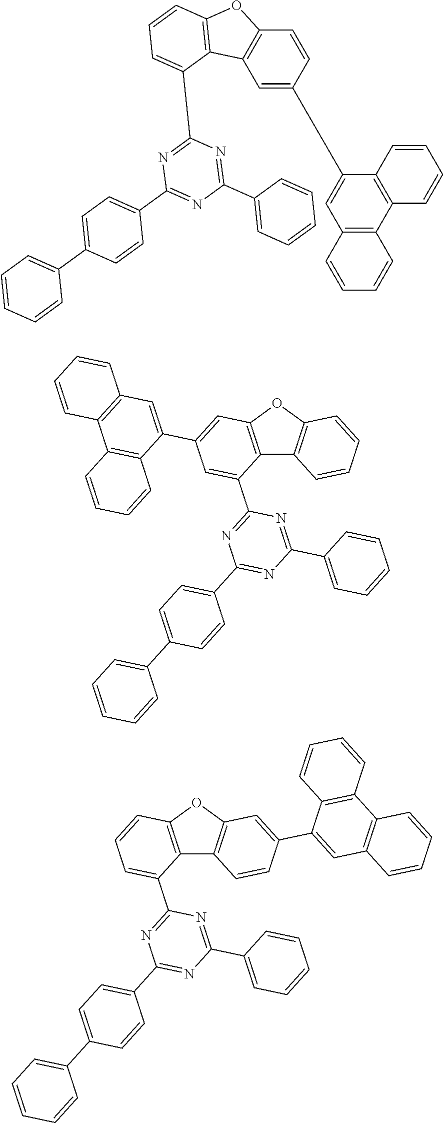 [SMILES: c1ccc(-c2ccc(-c3nc(Cc4cccc5oc6ccc(CCc7cc8ccccc8c8ccccc78)cc6c45)nc(-c4ccccc4)n3)cc2)cc1 ; c1ccc(-c2ccc(-c3nc(-c4ccccc4)nc(-c4cc(-c5cc6ccccc6c6ccccc56)cc5oc6ccccc6c45)n3)cc2)cc1 ; c1ccc(-c2ccc(-c3nc(-c4ccccc4)nc(-c4cccc5oc6cc(-c7cc8ccccc8c8ccccc78)ccc6c45)n3)cc2)cc1]

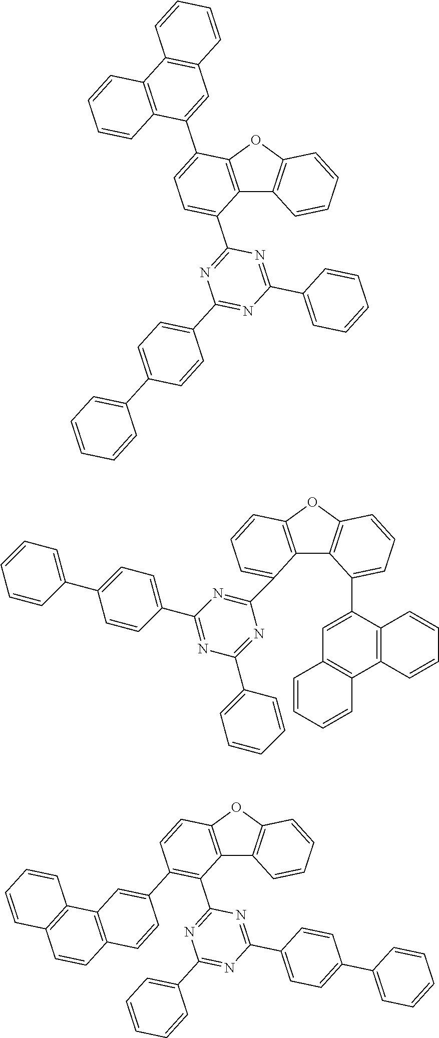 [SMILES: c1ccc(-c2ccc(-c3nc(-c4ccccc4)nc(-c4ccc(-c5cc6ccccc6c6ccccc56)c5oc6ccccc6c45)n3)cc2)cc1 ; c1ccc(-c2ccc(-c3nc(-c4ccccc4)nc(-c4cccc5oc6cccc(-c7cc8ccccc8c8ccccc78)c6c45)n3)cc2)cc1 ; c1ccc(-c2ccc(-c3nc(-c4ccccc4)nc(-c4c(-c5ccc6ccc7ccccc7c6c5)ccc5oc6ccccc6c45)n3)cc2)cc1]

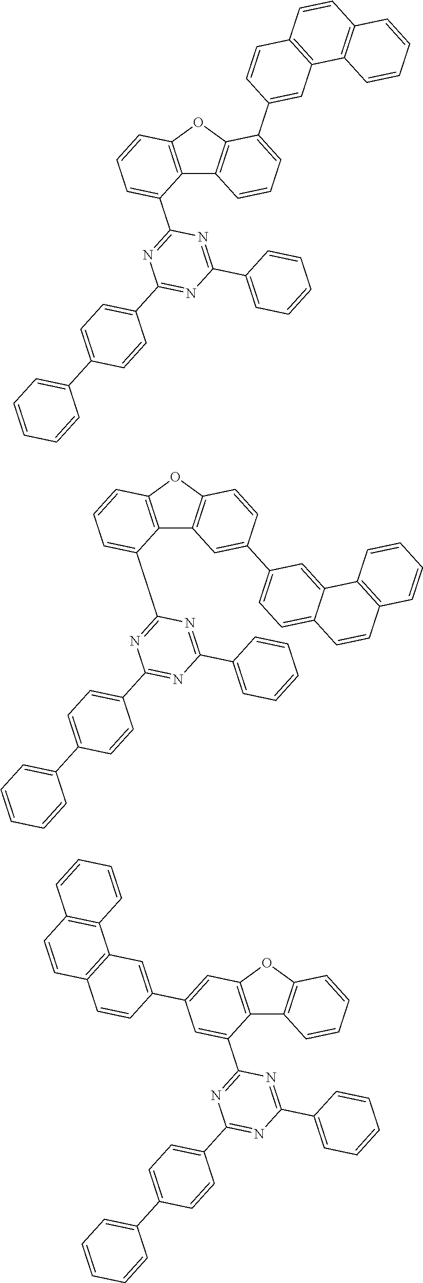 [SMILES: c1ccc(-c2ccc(-c3nc(-c4ccccc4)nc(-c4cccc5oc6c(-c7ccc8ccc9ccccc9c8c7)cccc6c45)n3)cc2)cc1 ; c1ccc(-c2ccc(-c3nc(Cc4cccc5oc6ccc(-c7ccc8ccc9ccccc9c8c7)cc6c45)nc(-c4ccccc4)n3)cc2)cc1 ; c1ccc(-c2ccc(-c3nc(-c4ccccc4)nc(-c4cc(-c5ccc6ccc7ccccc7c6c5)cc5oc6ccccc6c45)n3)cc2)cc1]

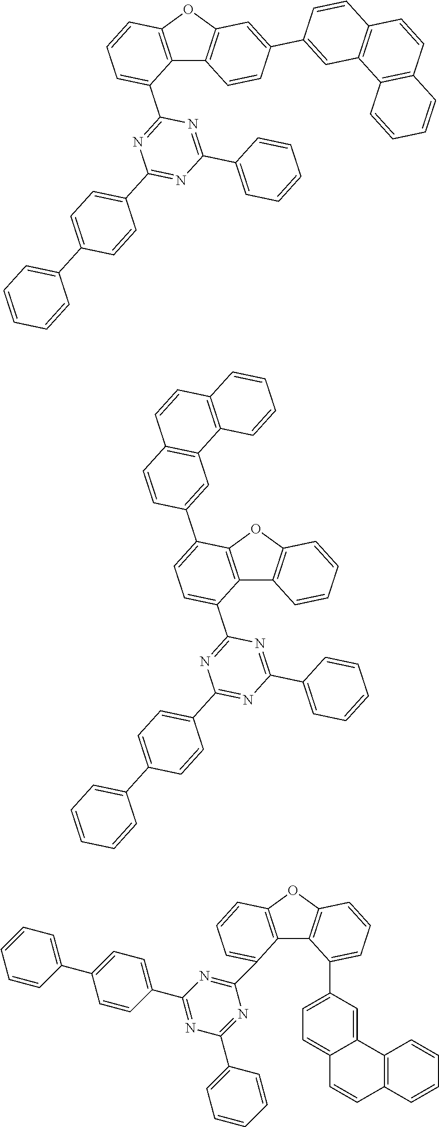 [SMILES: c1ccc(-c2ccc(-c3nc(-c4ccccc4)nc(-c4cccc5oc6cc(-c7ccc8ccc9ccccc9c8c7)ccc6c45)n3)cc2)cc1 ; c1ccc(-c2ccc(-c3nc(-c4ccccc4)nc(-c4ccc(-c5ccc6ccc7ccccc7c6c5)c5oc6ccccc6c45)n3)cc2)cc1 ; c1ccc(-c2ccc(-c3nc(-c4ccccc4)nc(-c4cccc5oc6cccc(-c7ccc8ccc9ccccc9c8c7)c6c45)n3)cc2)cc1]

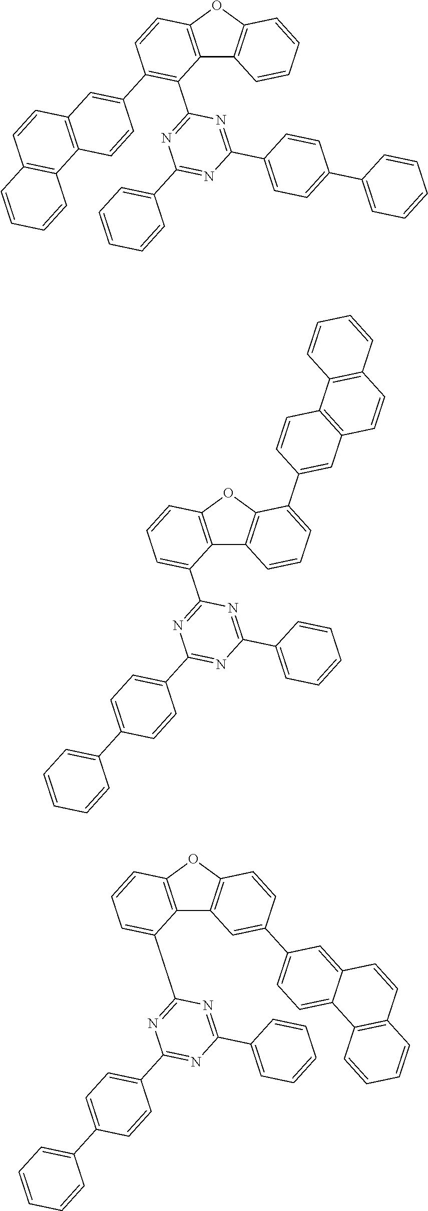 [SMILES: c1ccc(-c2ccc(-c3nc(-c4ccccc4)nc(-c4c(-c5ccc6c(ccc7ccccc76)c5)ccc5oc6ccccc6c45)n3)cc2)cc1 ; c1ccc(-c2ccc(-c3nc(-c4ccccc4)nc(-c4cccc5oc6c(-c7ccc8c(ccc9ccccc98)c7)cccc6c45)n3)cc2)cc1 ; c1ccc(-c2ccc(-c3nc(Cc4cccc5oc6ccc(-c7ccc8c(ccc9ccccc98)c7)cc6c45)nc(-c4ccccc4)n3)cc2)cc1]

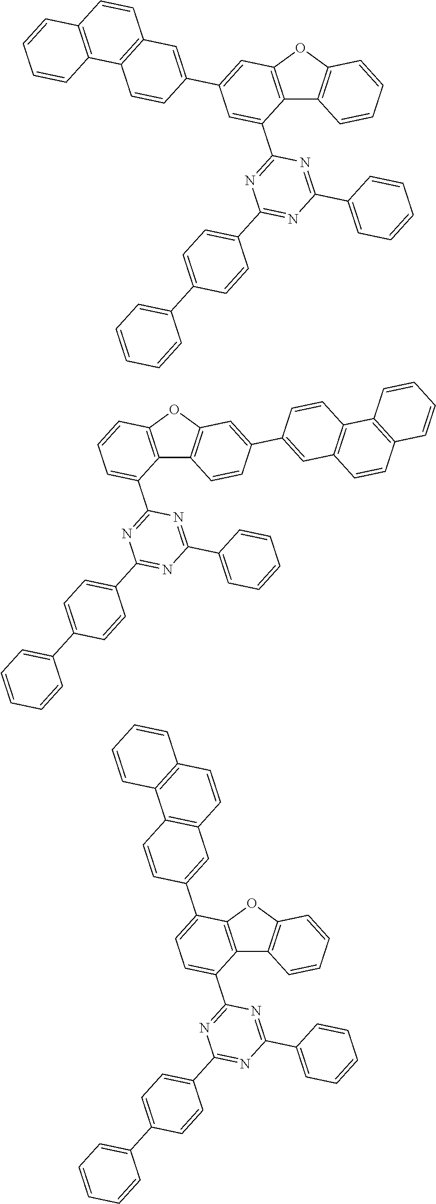 [SMILES: c1ccc(-c2ccc(-c3nc(-c4ccccc4)nc(-c4cc(-c5ccc6c(ccc7ccccc76)c5)cc5oc6ccccc6c45)n3)cc2)cc1 ; c1ccc(-c2ccc(-c3nc(-c4ccccc4)nc(-c4cccc5oc6cc(-c7ccc8c(ccc9ccccc98)c7)ccc6c45)n3)cc2)cc1 ; c1ccc(-c2ccc(-c3nc(-c4ccccc4)nc(-c4ccc(-c5ccc6c(ccc7ccccc76)c5)c5oc6ccccc6c45)n3)cc2)cc1]

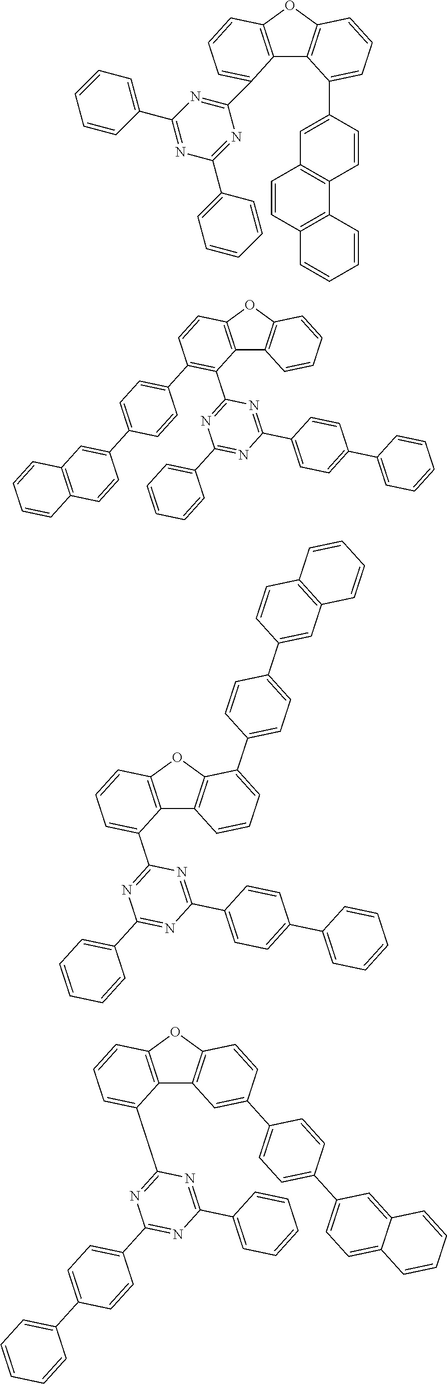

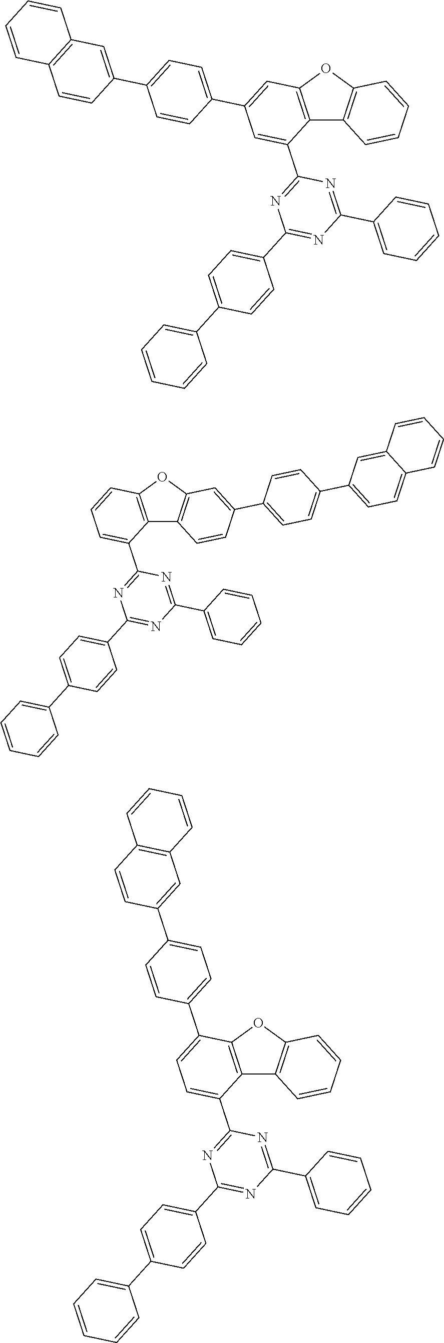 [SMILES: c1ccc(-c2ccc(-c3nc(-c4ccccc4)nc(-c4cc(-c5ccc(-c6ccc7ccccc7c6)cc5)cc5oc6ccccc6c45)n3)cc2)cc1 ; c1ccc(-c2ccc(-c3nc(-c4ccccc4)nc(-c4cccc5oc6cc(-c7ccc(-c8ccc9ccccc9c8)cc7)ccc6c45)n3)cc2)cc1 ; c1ccc(-c2ccc(-c3nc(-c4ccccc4)nc(-c4ccc(-c5ccc(-c6ccc7ccccc7c6)cc5)c5oc6ccccc6c45)n3)cc2)cc1]

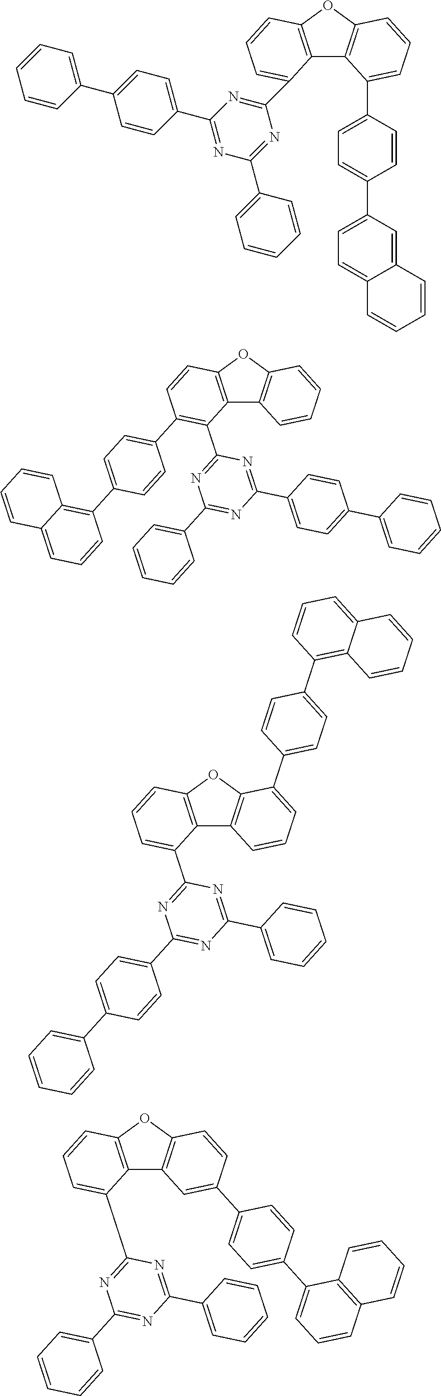

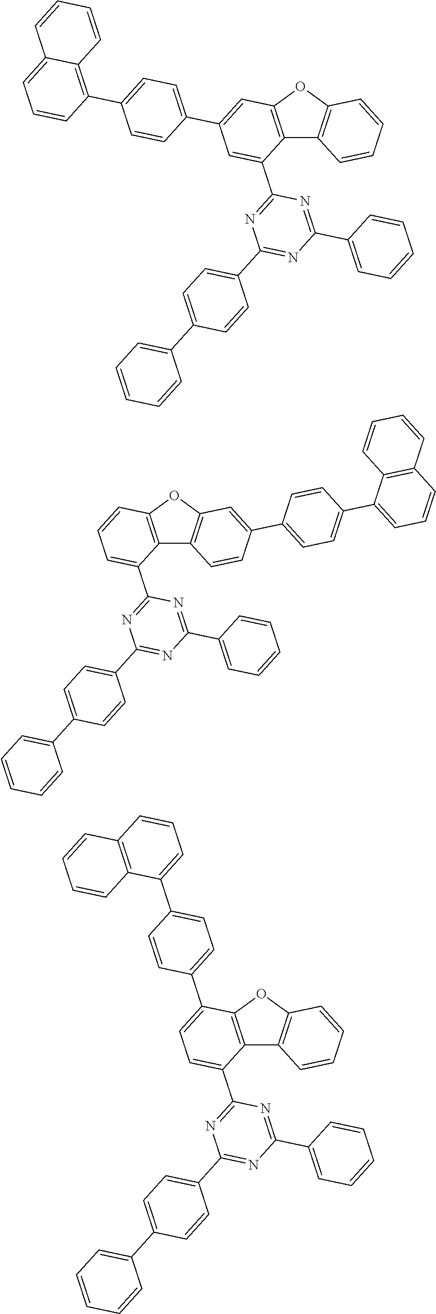 [SMILES: c1ccc(-c2ccc(-c3nc(-c4ccccc4)nc(-c4cc(-c5ccc(-c6cccc7ccccc67)cc5)cc5oc6ccccc6c45)n3)cc2)cc1 ; c1ccc(-c2ccc(-c3nc(-c4ccccc4)nc(-c4cccc5oc6cc(-c7ccc(-c8cccc9ccccc89)cc7)ccc6c45)n3)cc2)cc1 ; c1ccc(-c2ccc(-c3nc(-c4ccccc4)nc(-c4ccc(-c5ccc(-c6cccc7ccccc67)cc5)c5oc6ccccc6c45)n3)cc2)cc1]

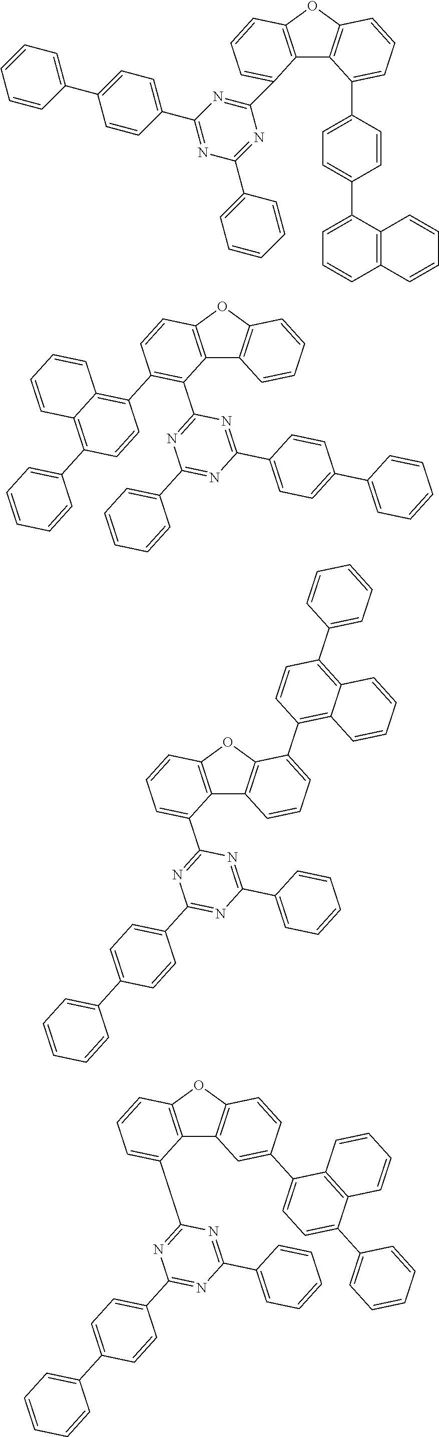 [SMILES: c1ccc(-c2ccc(-c3nc(-c4ccccc4)nc(-c4cccc5oc6cccc(-c7ccc(-c8cccc9ccccc89)cc7)c6c45)n3)cc2)cc1 ; c1ccc(-c2ccc(-c3nc(-c4ccccc4)nc(-c4c(-c5ccc(-c6ccccc6)c6ccccc56)ccc5oc6ccccc6c45)n3)cc2)cc1 ; c1ccc(-c2ccc(-c3nc(-c4ccccc4)nc(-c4cccc5oc6c(-c7ccc(-c8ccccc8)c8ccccc78)cccc6c45)n3)cc2)cc1 ; c1ccc(-c2ccc(-c3nc(Cc4cccc5oc6ccc(-c7ccc(-c8ccccc8)c8ccccc78)cc6c45)nc(-c4ccccc4)n3)cc2)cc1]

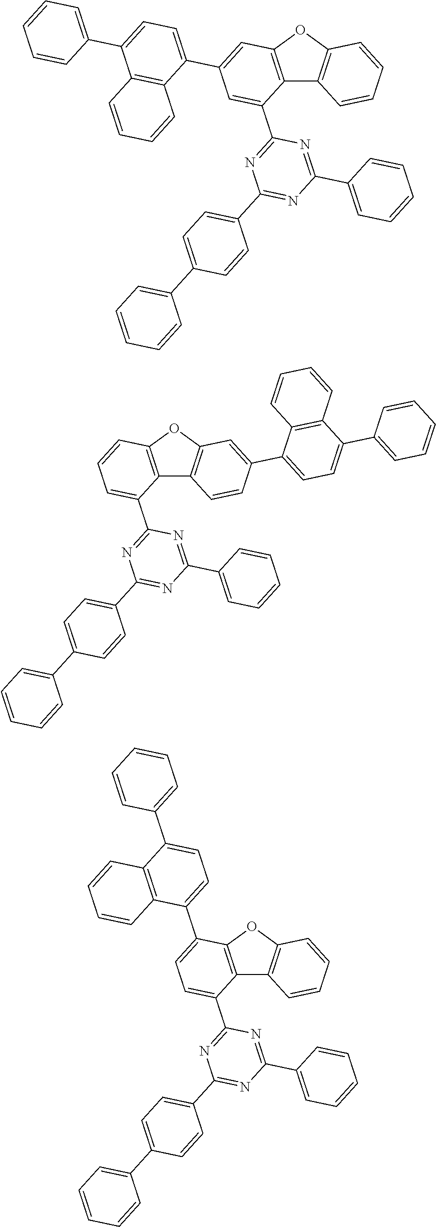 [SMILES: c1ccc(-c2ccc(-c3nc(-c4ccccc4)nc(-c4cc(-c5ccc(-c6ccccc6)c6ccccc56)cc5oc6ccccc6c45)n3)cc2)cc1 ; c1ccc(-c2ccc(-c3nc(-c4ccccc4)nc(-c4cccc5oc6cc(-c7ccc(-c8ccccc8)c8ccccc78)ccc6c45)n3)cc2)cc1 ; c1ccc(-c2ccc(-c3nc(-c4ccccc4)nc(-c4ccc(-c5ccc(-c6ccccc6)c6ccccc56)c5oc6ccccc6c45)n3)cc2)cc1]

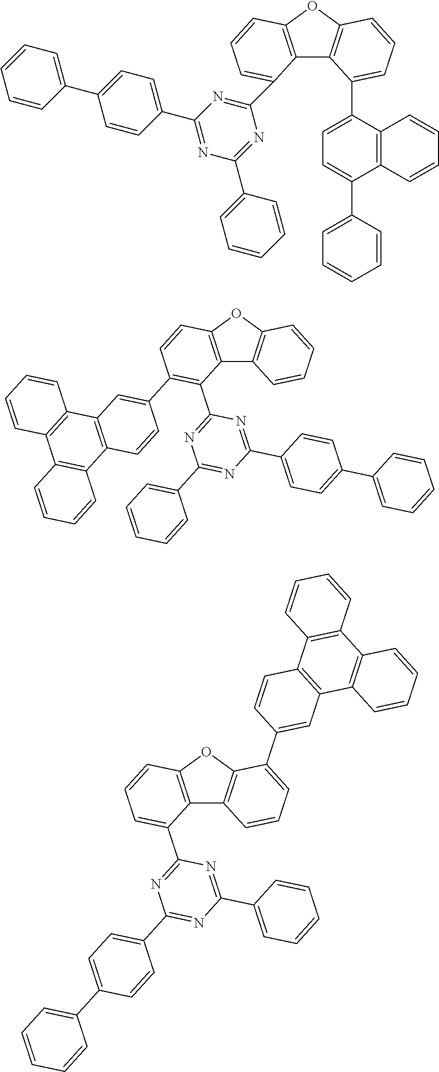 [SMILES: c1ccc(-c2ccc(-c3nc(-c4ccccc4)nc(-c4cccc5oc6cccc(-c7ccc(-c8ccccc8)c8ccccc78)c6c45)n3)cc2)cc1 ; c1ccc(-c2ccc(-c3nc(-c4ccccc4)nc(-c4c(-c5ccc6c7ccccc7c7ccccc7c6c5)ccc5oc6ccccc6c45)n3)cc2)cc1 ; c1ccc(-c2ccc(-c3nc(-c4ccccc4)nc(-c4cccc5oc6c(-c7ccc8c9ccccc9c9ccccc9c8c7)cccc6c45)n3)cc2)cc1]

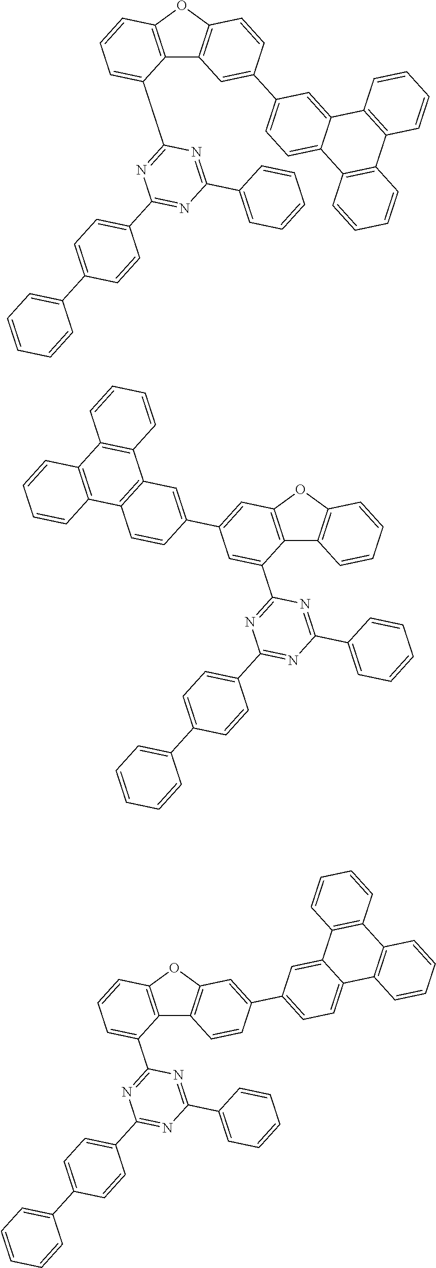 [SMILES: c1ccc(-c2ccc(-c3nc(Cc4cccc5oc6ccc(-c7ccc8c9ccccc9c9ccccc9c8c7)cc6c45)nc(-c4ccccc4)n3)cc2)cc1 ; c1ccc(-c2ccc(-c3nc(-c4ccccc4)nc(-c4cc(-c5ccc6c7ccccc7c7ccccc7c6c5)cc5oc6ccccc6c45)n3)cc2)cc1 ; c1ccc(-c2ccc(-c3nc(-c4ccccc4)nc(-c4cccc5oc6cc(-c7ccc8c9ccccc9c9ccccc9c8c7)ccc6c45)n3)cc2)cc1]

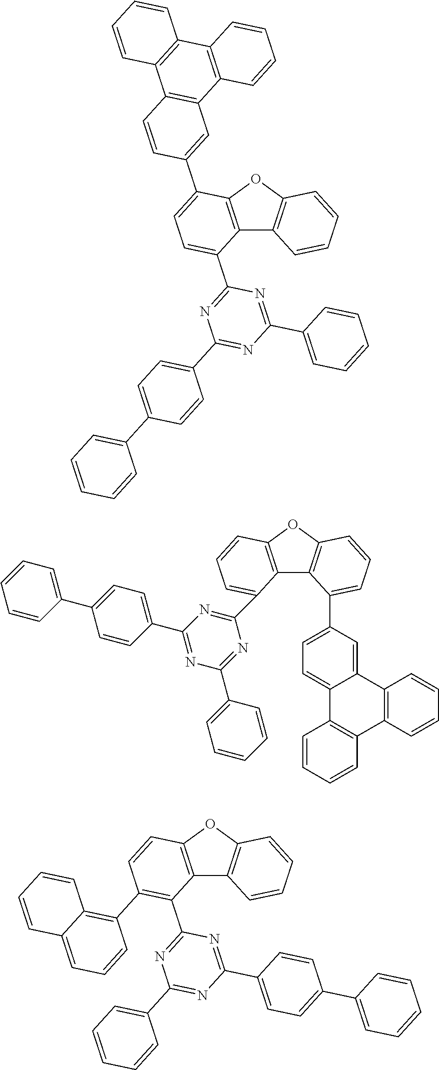 [SMILES: c1ccc(-c2ccc(-c3nc(-c4ccccc4)nc(-c4ccc(-c5ccc6c7ccccc7c7ccccc7c6c5)c5oc6ccccc6c45)n3)cc2)cc1 ; c1ccc(-c2ccc(-c3nc(-c4ccccc4)nc(-c4cccc5oc6cccc(-c7ccc8c9ccccc9c9ccccc9c8c7)c6c45)n3)cc2)cc1 ; c1ccc(-c2ccc(-c3nc(-c4ccccc4)nc(-c4c(-c5cccc6ccccc56)ccc5oc6ccccc6c45)n3)cc2)cc1]

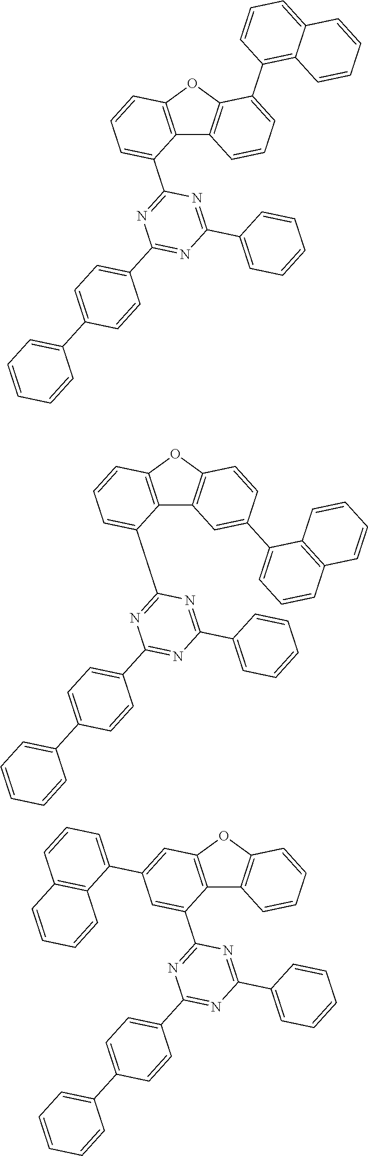

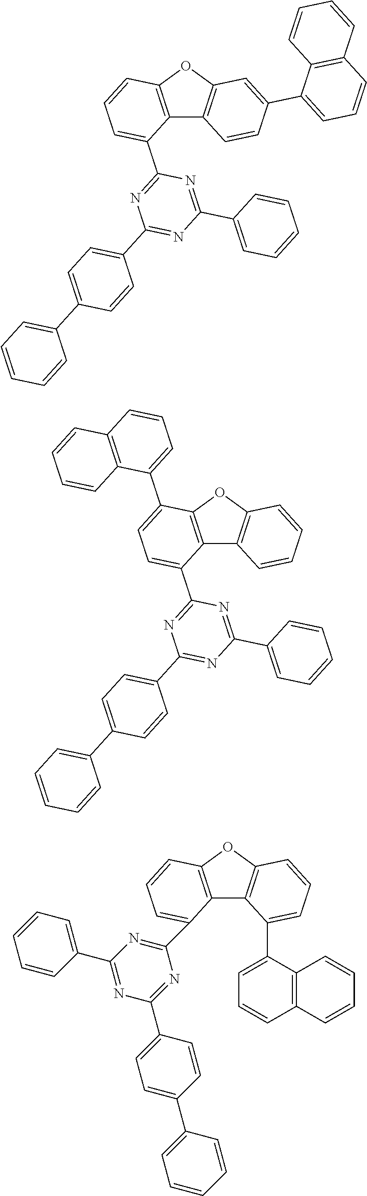 [SMILES: c1ccc(-c2ccc(-c3nc(-c4ccccc4)nc(-c4cccc5oc6cc(-c7cccc8ccccc78)ccc6c45)n3)cc2)cc1 ; c1ccc(-c2ccc(-c3nc(-c4ccccc4)nc(-c4ccc(-c5cccc6ccccc56)c5oc6ccccc6c45)n3)cc2)cc1 ; c1ccc(-c2ccc(-c3nc(-c4ccccc4)nc(-c4cccc5oc6cccc(-c7cccc8ccccc78)c6c45)n3)cc2)cc1]

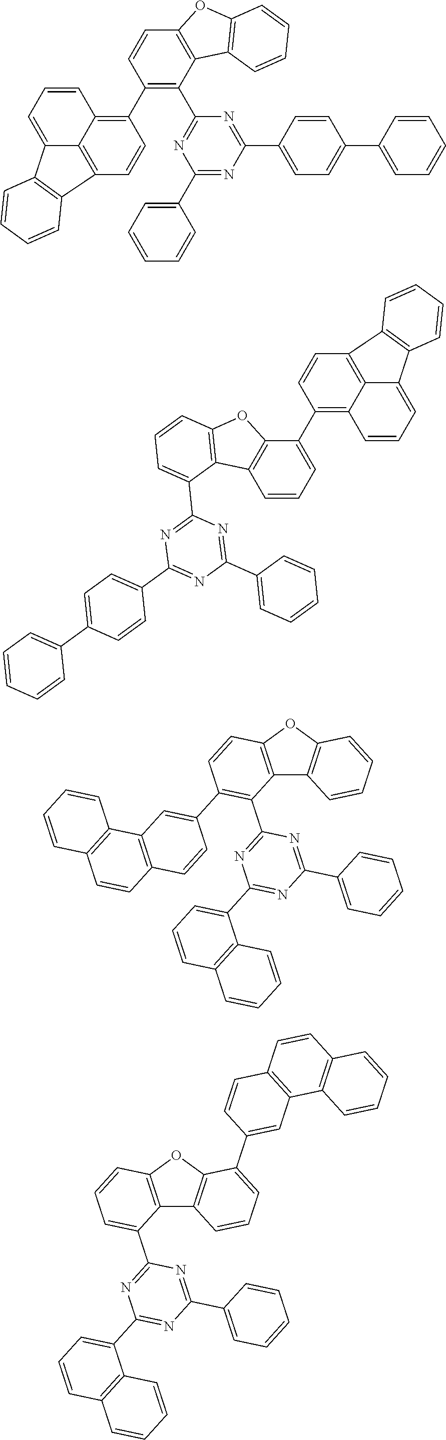

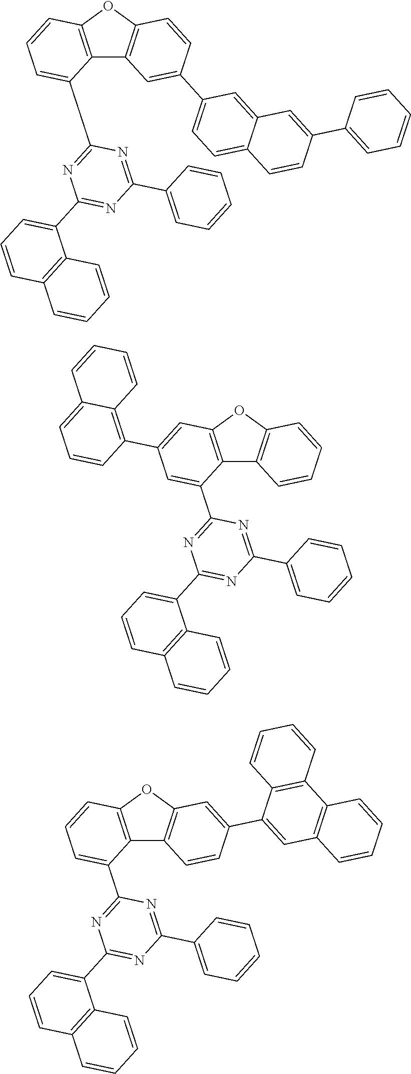

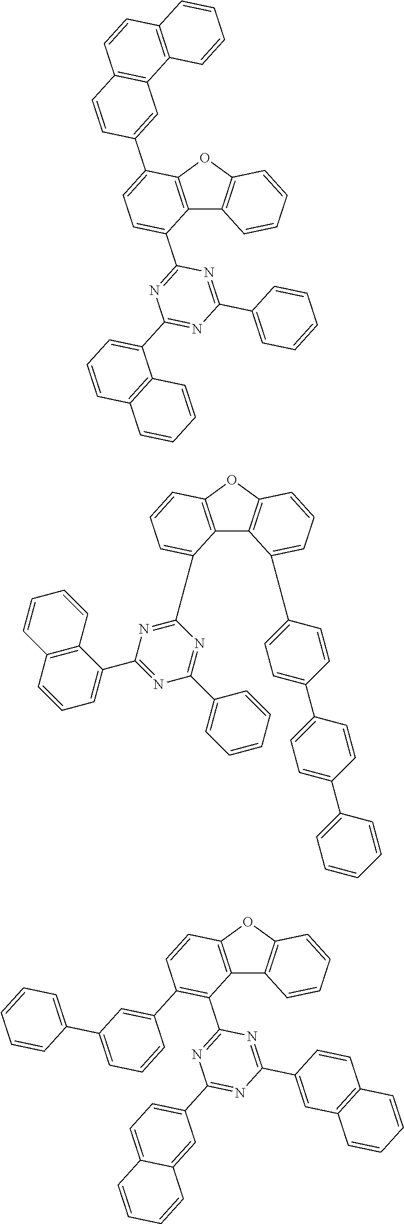 [SMILES: c1ccc(-c2nc(-c3cccc4ccccc34)nc(-c3ccc(-c4ccc5ccc6ccccc6c5c4)c4oc5ccccc5c34)n2)cc1 ; c1ccc(-c2ccc(-c3ccc(Cc4cccc5oc6cccc(Cc7nc(-c8ccccc8)nc(-c8cccc9ccccc89)n7)c6c45)cc3)cc2)cc1 ; c1ccc(-c2cccc(-c3ccc4oc5ccccc5c4c3-c3nc(-c4ccc5ccccc5c4)nc(-c4ccc5ccccc5c4)n3)c2)cc1]

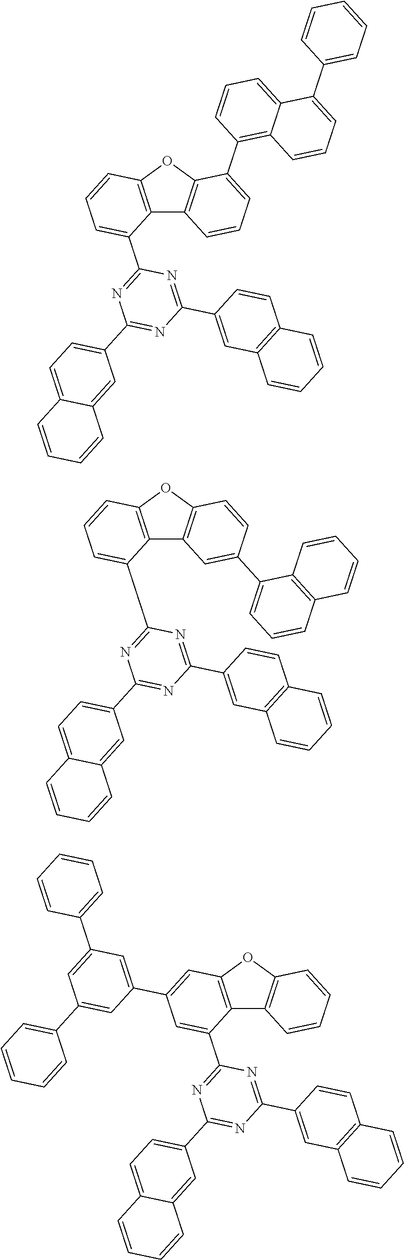

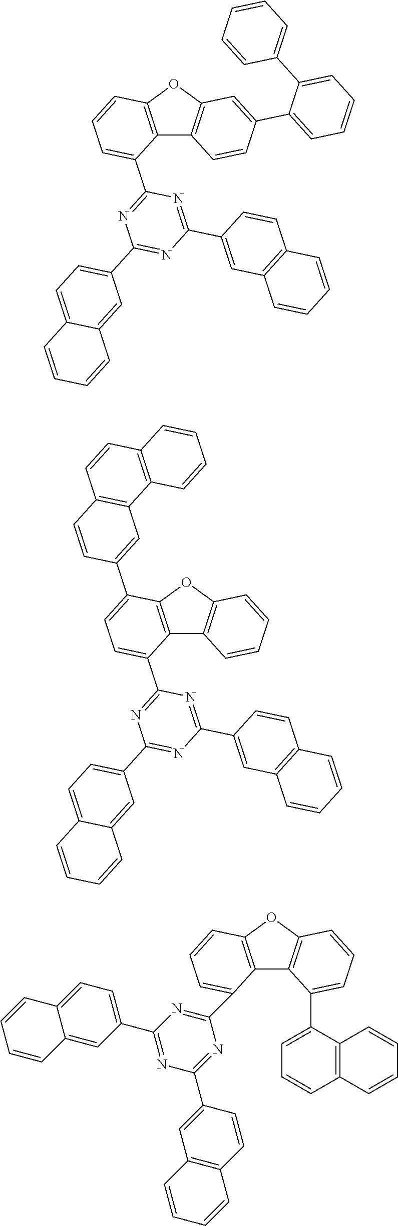 [SMILES: c1ccc(-c2ccccc2-c2ccc3c(c2)oc2cccc(-c4nc(-c5ccc6ccccc6c5)nc(-c5ccc6ccccc6c5)n4)c23)cc1 ; c1ccc2cc(-c3nc(-c4ccc5ccccc5c4)nc(-c4ccc(-c5ccc6ccc7ccccc7c6c5)c5oc6ccccc6c45)n3)ccc2c1 ; c1ccc2cc(-c3nc(-c4ccc5ccccc5c4)nc(-c4cccc5oc6cccc(-c7cccc8ccccc78)c6c45)n3)ccc2c1]

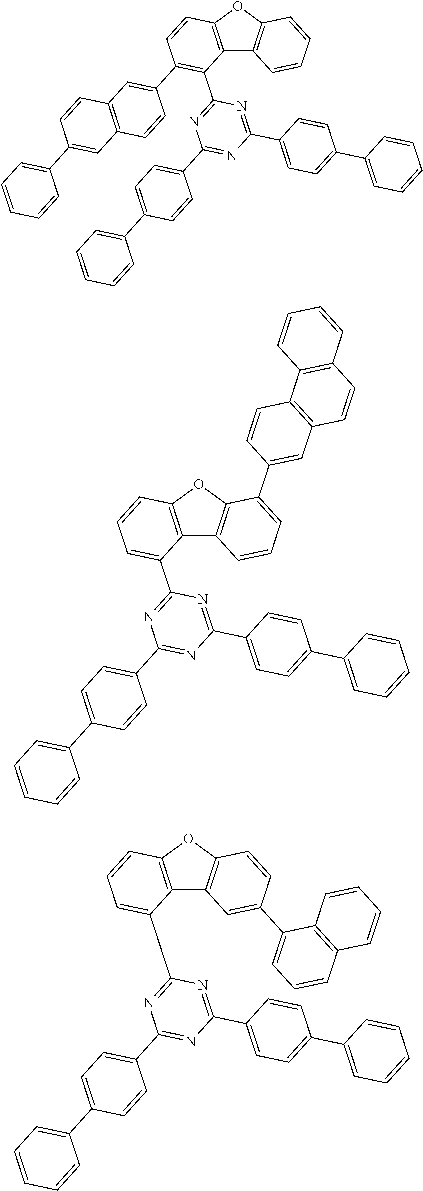 [SMILES: c1ccc(-c2ccc(-c3nc(-c4ccc(-c5ccccc5)cc4)nc(-c4c(-c5ccc6cc(-c7ccccc7)ccc6c5)ccc5oc6ccccc6c45)n3)cc2)cc1 ; c1ccc(-c2ccc(-c3nc(-c4ccc(-c5ccccc5)cc4)nc(-c4cccc5oc6c(-c7ccc8c(ccc9ccccc98)c7)cccc6c45)n3)cc2)cc1 ; c1ccc(-c2ccc(-c3nc(Cc4cccc5oc6ccc(-c7cccc8ccccc78)cc6c45)nc(-c4ccc(-c5ccccc5)cc4)n3)cc2)cc1]

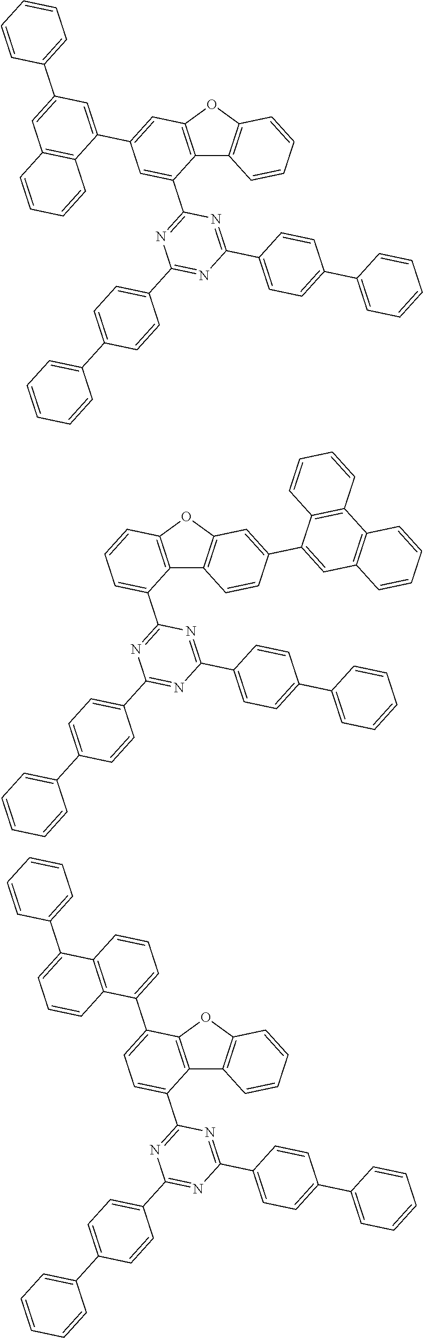

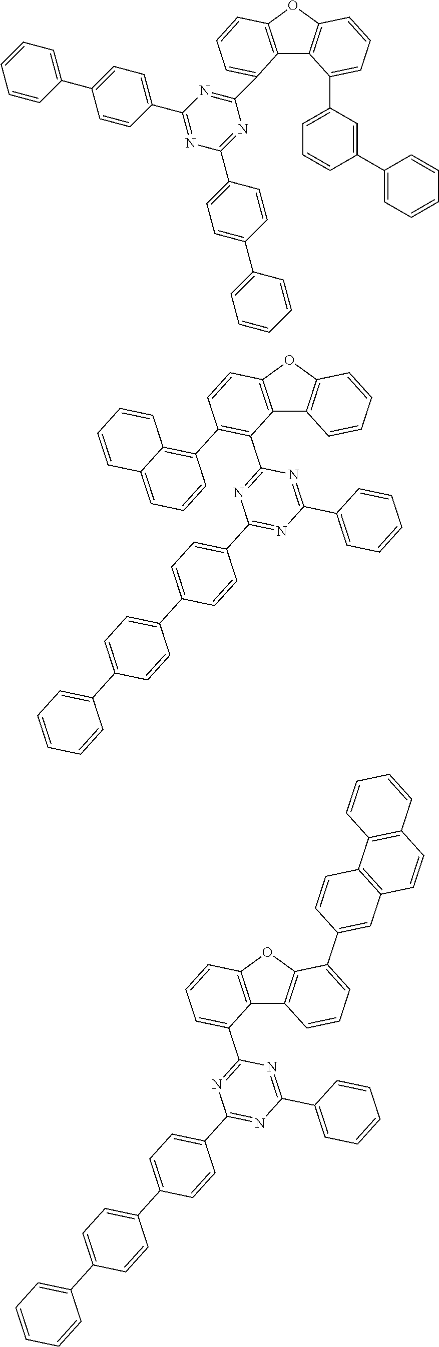

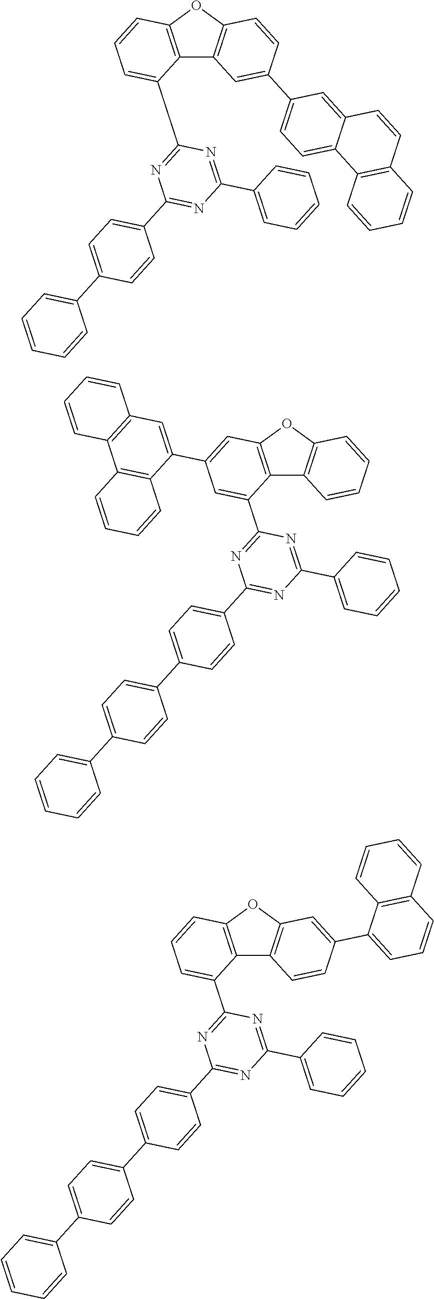

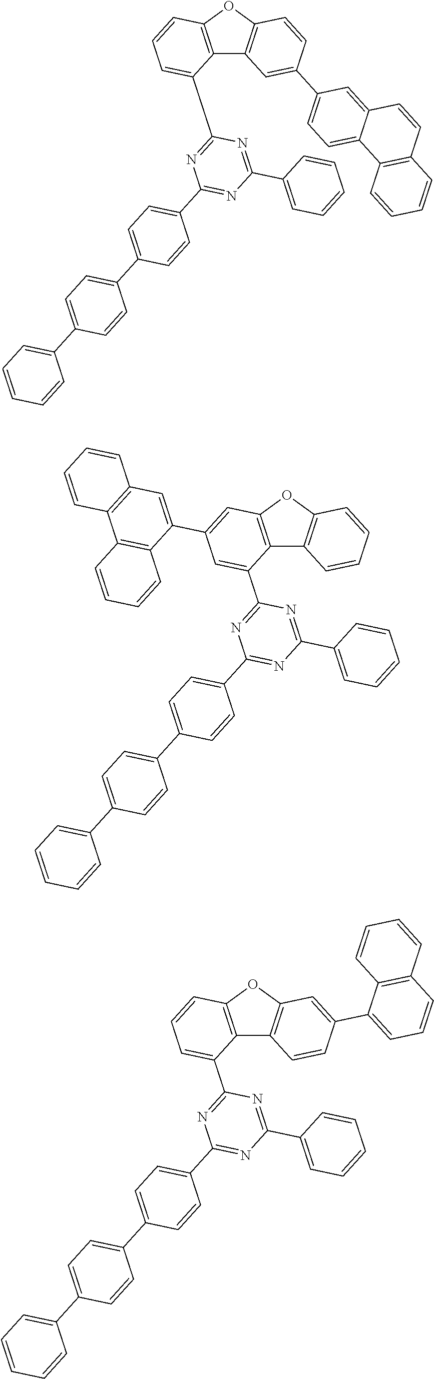 [SMILES: c1ccc(-c2ccc(-c3ccc(-c4nc(Cc5cccc6oc7ccc(-c8ccc9c(ccc%10ccccc%109)c8)cc7c56)nc(-c5ccccc5)n4)cc3)cc2)cc1 ; c1ccc(-c2ccc(-c3ccc(-c4nc(-c5ccccc5)nc(-c5cc(-c6cc7ccccc7c7ccccc67)cc6oc7ccccc7c56)n4)cc3)cc2)cc1 ; c1ccc(-c2ccc(-c3ccc(-c4nc(-c5ccccc5)nc(-c5cccc6oc7cc(-c8cccc9ccccc89)ccc7c56)n4)cc3)cc2)cc1]

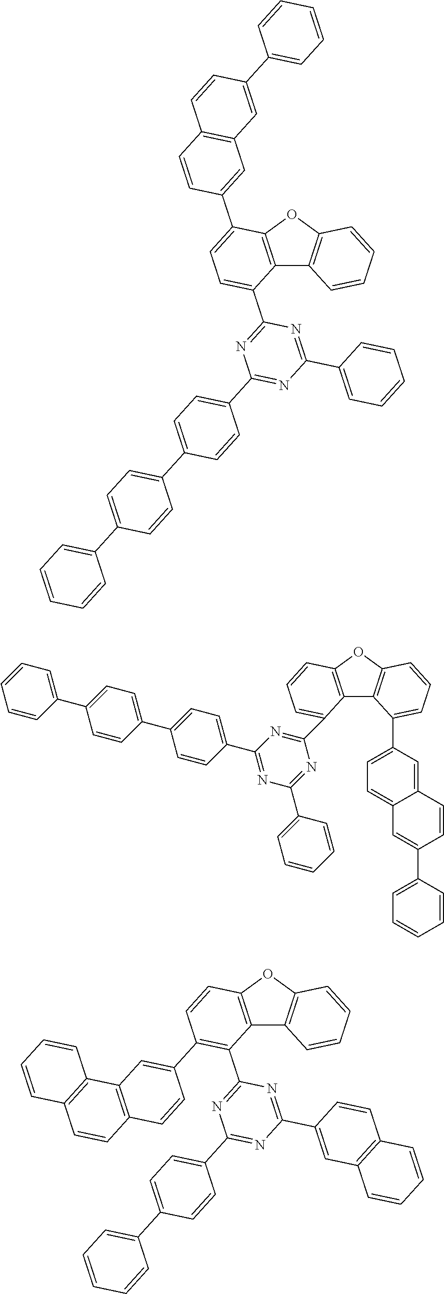 [SMILES: c1ccc(-c2ccc(-c3ccc(-c4nc(-c5ccccc5)nc(-c5ccc(-c6ccc7ccc(-c8ccccc8)cc7c6)c6oc7ccccc7c56)n4)cc3)cc2)cc1 ; c1ccc(-c2ccc(-c3ccc(-c4nc(-c5ccccc5)nc(-c5cccc6oc7cccc(-c8ccc9cc(-c%10ccccc%10)ccc9c8)c7c56)n4)cc3)cc2)cc1 ; c1ccc(-c2ccc(-c3nc(-c4ccc5ccccc5c4)nc(-c4c(-c5ccc6ccc7ccccc7c6c5)ccc5oc6ccccc6c45)n3)cc2)cc1]

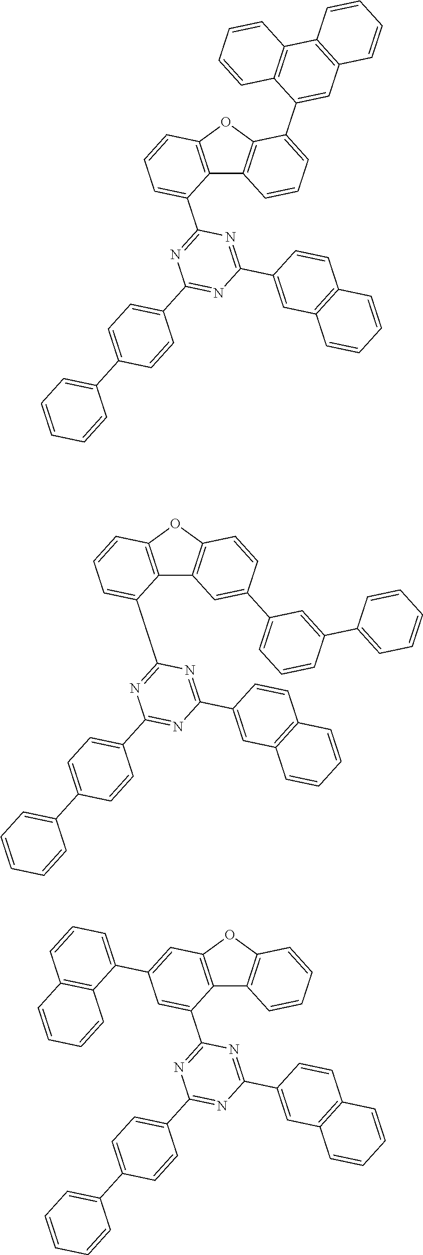 [SMILES: c1ccc(-c2ccc(-c3nc(-c4ccc5ccccc5c4)nc(-c4cccc5oc6c(-c7cc8ccccc8c8ccccc78)cccc6c45)n3)cc2)cc1 ; c1ccc(-c2ccc(-c3nc(Cc4cccc5oc6ccc(-c7cccc(-c8ccccc8)c7)cc6c45)nc(-c4ccc5ccccc5c4)n3)cc2)cc1 ; c1ccc(-c2ccc(-c3nc(-c4ccc5ccccc5c4)nc(-c4cc(-c5cccc6ccccc56)cc5oc6ccccc6c45)n3)cc2)cc1]

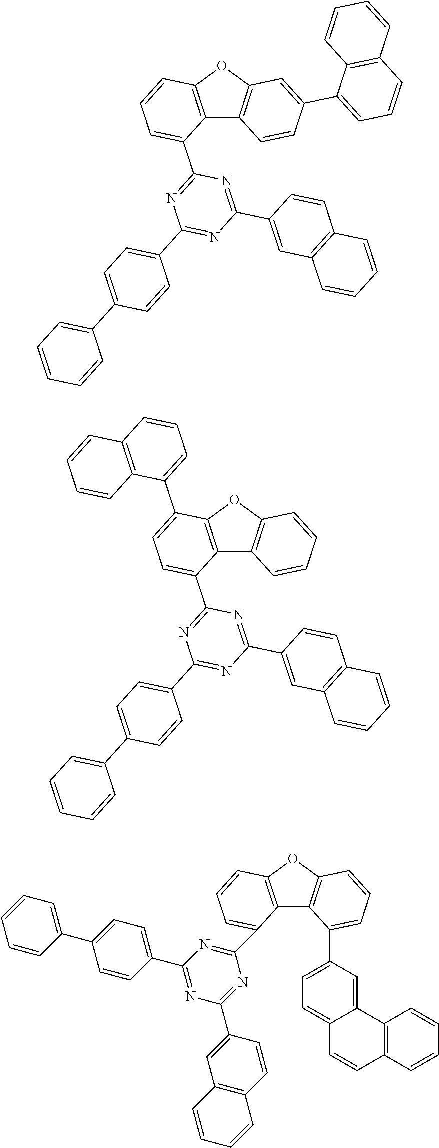 [SMILES: c1ccc(-c2ccc(-c3nc(-c4ccc5ccccc5c4)nc(-c4cccc5oc6cc(-c7cccc8ccccc78)ccc6c45)n3)cc2)cc1 ; c1ccc(-c2ccc(-c3nc(-c4ccc5ccccc5c4)nc(-c4ccc(-c5cccc6ccccc56)c5oc6ccccc6c45)n3)cc2)cc1 ; c1ccc(-c2ccc(-c3nc(-c4ccc5ccccc5c4)nc(-c4cccc5oc6cccc(-c7ccc8ccc9ccccc9c8c7)c6c45)n3)cc2)cc1]

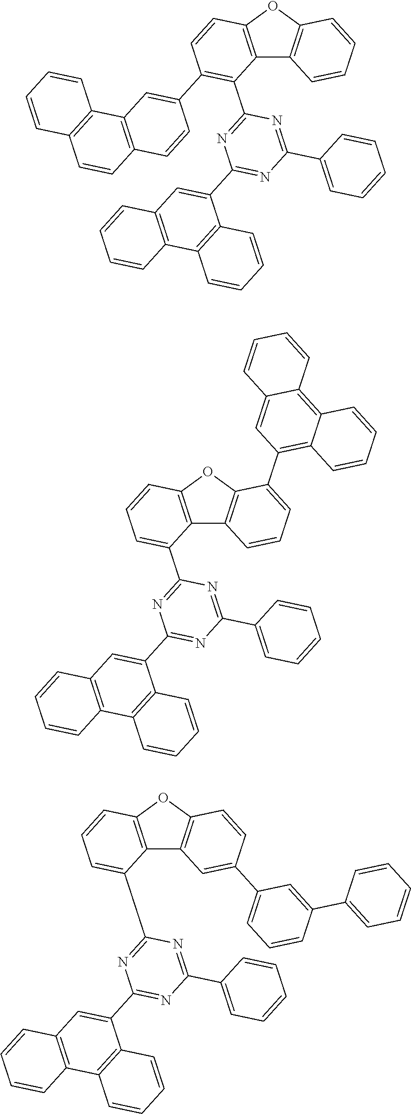

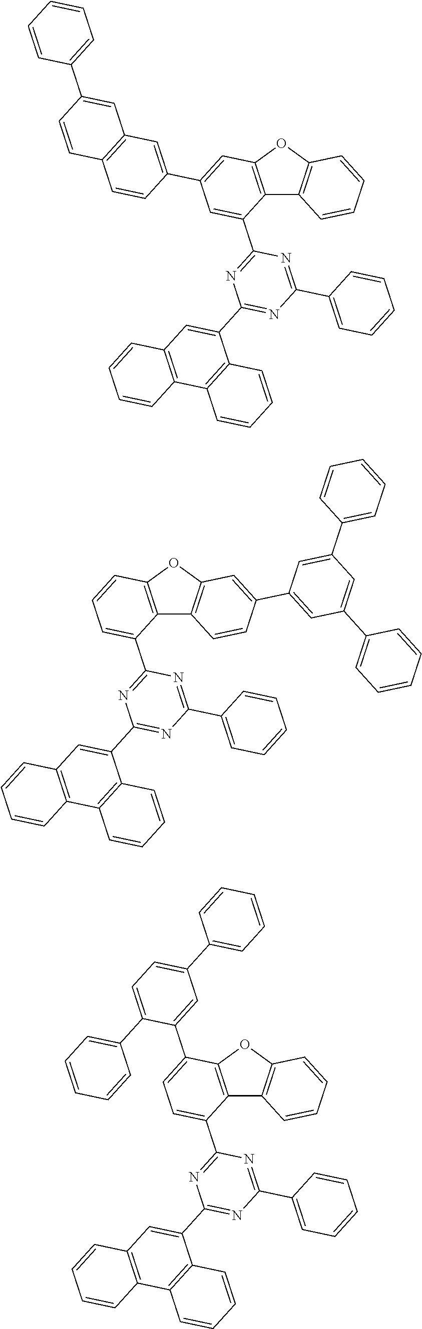 [SMILES: c1ccc(-c2ccc3ccc(-c4cc(-c5nc(-c6ccccc6)nc(-c6cc7ccccc7c7ccccc67)n5)c5c(c4)oc4ccccc45)cc3c2)cc1 ; c1ccc(-c2cc(-c3ccccc3)cc(-c3ccc4c(c3)oc3cccc(-c5nc(-c6ccccc6)nc(-c6cc7ccccc7c7ccccc67)n5)c34)c2)cc1 ; c1ccc(-c2ccc(-c3ccccc3)c(-c3ccc(-c4nc(-c5ccccc5)nc(-c5cc6ccccc6c6ccccc56)n4)c4c3oc3ccccc34)c2)cc1]

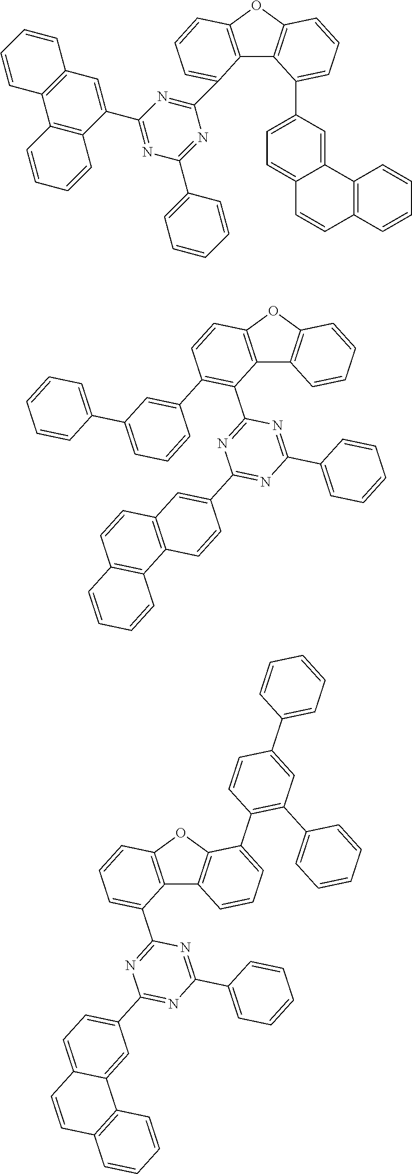 [SMILES: c1ccc(-c2nc(-c3cc4ccccc4c4ccccc34)nc(-c3cccc4oc5cccc(-c6ccc7ccc8ccccc8c7c6)c5c34)n2)cc1 ; c1ccc(-c2cccc(-c3ccc4oc5ccccc5c4c3-c3nc(-c4ccccc4)nc(-c4ccc5c(ccc6ccccc65)c4)n3)c2)cc1 ; c1ccc(-c2ccc(-c3cccc4c3oc3cccc(-c5nc(-c6ccccc6)nc(-c6ccc7ccc8ccccc8c7c6)n5)c34)c(-c3ccccc3)c2)cc1]

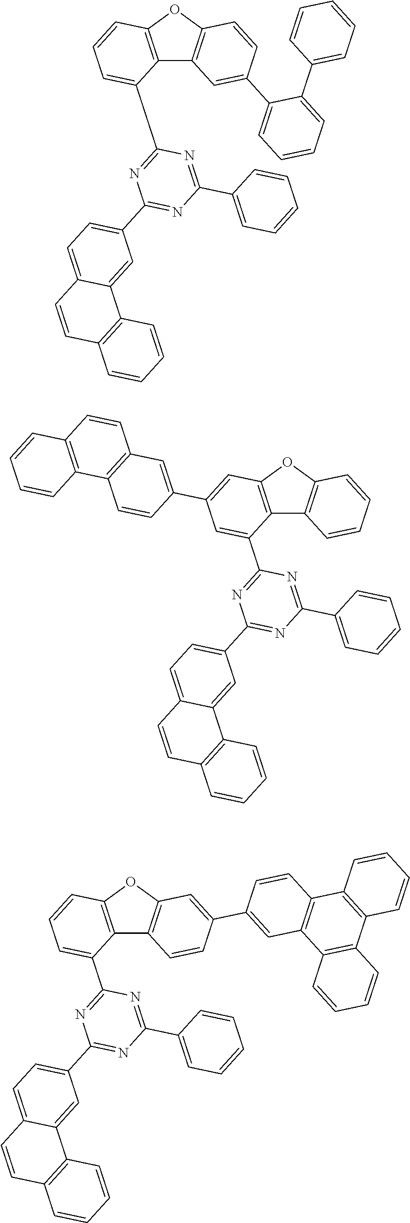

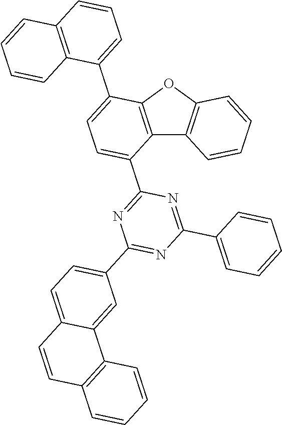 [SMILES: c1ccc(-c2nc(-c3ccc4ccc5ccccc5c4c3)nc(-c3ccc(-c4cccc5ccccc45)c4oc5ccccc5c34)n2)cc1]

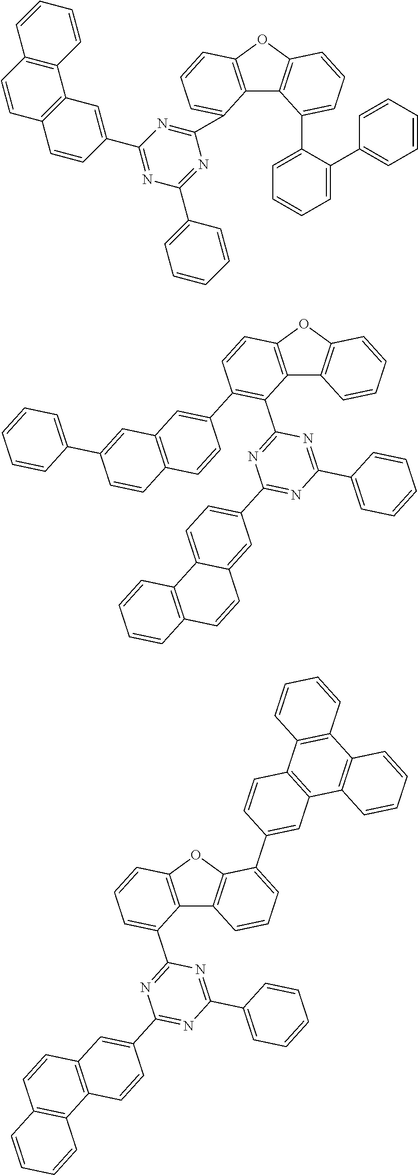 [SMILES: c1ccc(-c2nc(-c3ccc4ccc5ccccc5c4c3)nc(-c3cccc4oc5cccc(-c6ccccc6-c6ccccc6)c5c34)n2)cc1 ; c1ccc(-c2ccc3ccc(-c4ccc5oc6ccccc6c5c4-c4nc(-c5ccccc5)nc(-c5ccc6c(ccc7ccccc76)c5)n4)cc3c2)cc1 ; c1ccc(-c2nc(-c3ccc4c(ccc5ccccc54)c3)nc(-c3cccc4oc5c(-c6ccc7c8ccccc8c8ccccc8c7c6)cccc5c34)n2)cc1]

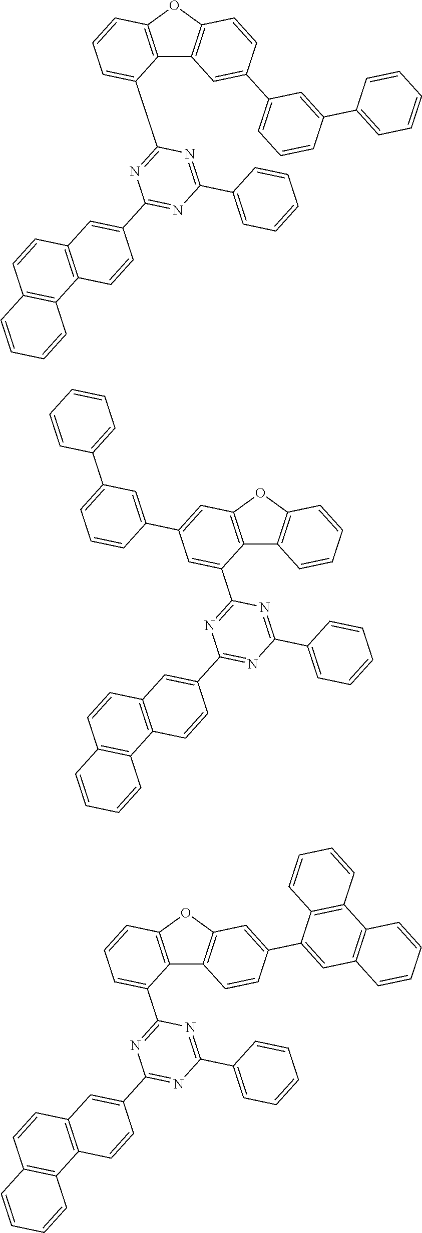

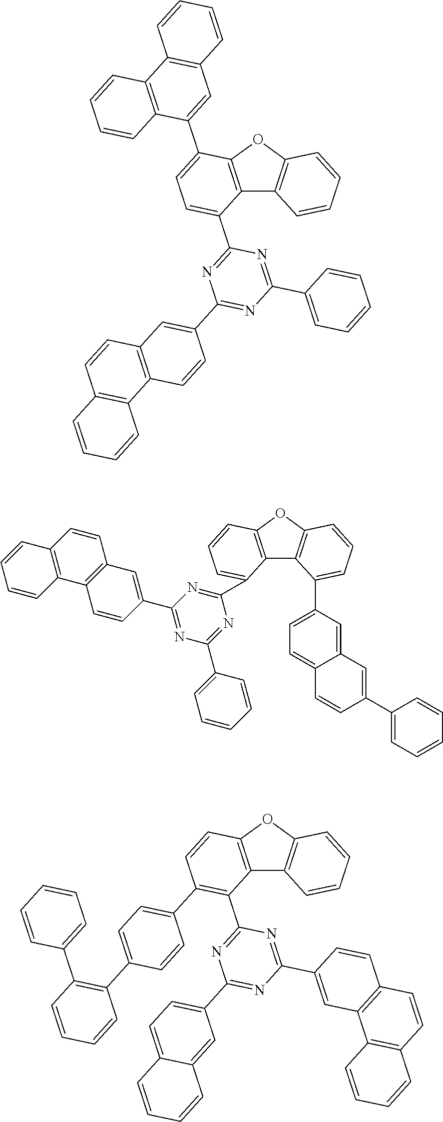 [SMILES: c1ccc(-c2nc(-c3ccc4c(ccc5ccccc54)c3)nc(-c3ccc(-c4cc5ccccc5c5ccccc45)c4oc5ccccc5c34)n2)cc1 ; c1ccc(-c2ccc3ccc(-c4cccc5oc6cccc(-c7nc(-c8ccccc8)nc(-c8ccc9c(ccc%10ccccc%109)c8)n7)c6c45)cc3c2)cc1 ; c1ccc(-c2ccccc2-c2ccc(-c3ccc4oc5ccccc5c4c3-c3nc(-c4ccc5ccccc5c4)nc(-c4ccc5ccc6ccccc6c5c4)n3)cc2)cc1]

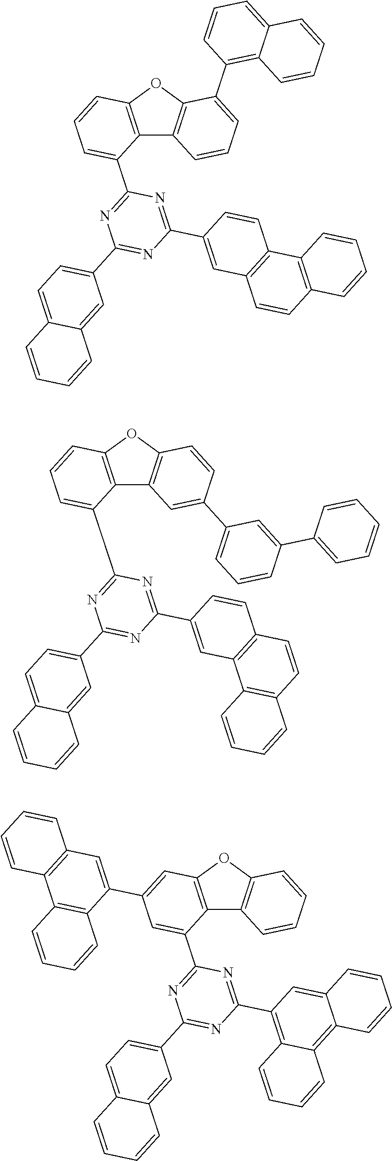 [SMILES: c1ccc2cc(-c3nc(-c4ccc5c(ccc6ccccc65)c4)nc(-c4cccc5oc6c(-c7cccc8ccccc78)cccc6c45)n3)ccc2c1 ; c1ccc(-c2cccc(-c3ccc4oc5cccc(-c6nc(-c7ccc8ccccc8c7)nc(-c7ccc8ccc9ccccc9c8c7)n6)c5c4c3)c2)cc1 ; c1ccc2cc(-c3nc(-c4cc5ccccc5c5ccccc45)nc(-c4cc(-c5cc6ccccc6c6ccccc56)cc5oc6ccccc6c45)n3)ccc2c1]

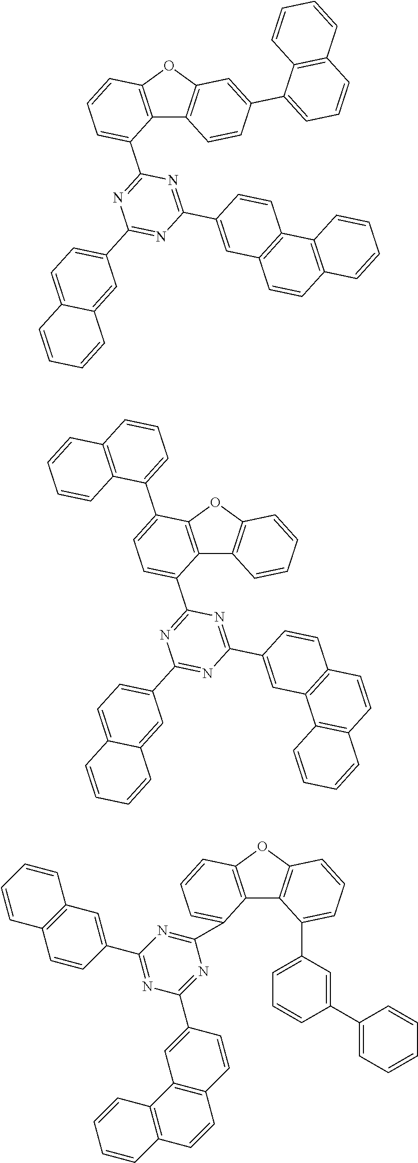 [SMILES: c1ccc2cc(-c3nc(-c4ccc5c(ccc6ccccc65)c4)nc(-c4cccc5oc6cc(-c7cccc8ccccc78)ccc6c45)n3)ccc2c1 ; c1ccc2cc(-c3nc(-c4ccc5ccc6ccccc6c5c4)nc(-c4ccc(-c5cccc6ccccc56)c5oc6ccccc6c45)n3)ccc2c1 ; c1ccc(-c2cccc(-c3cccc4oc5cccc(-c6nc(-c7ccc8ccccc8c7)nc(-c7ccc8ccc9ccccc9c8c7)n6)c5c34)c2)cc1]

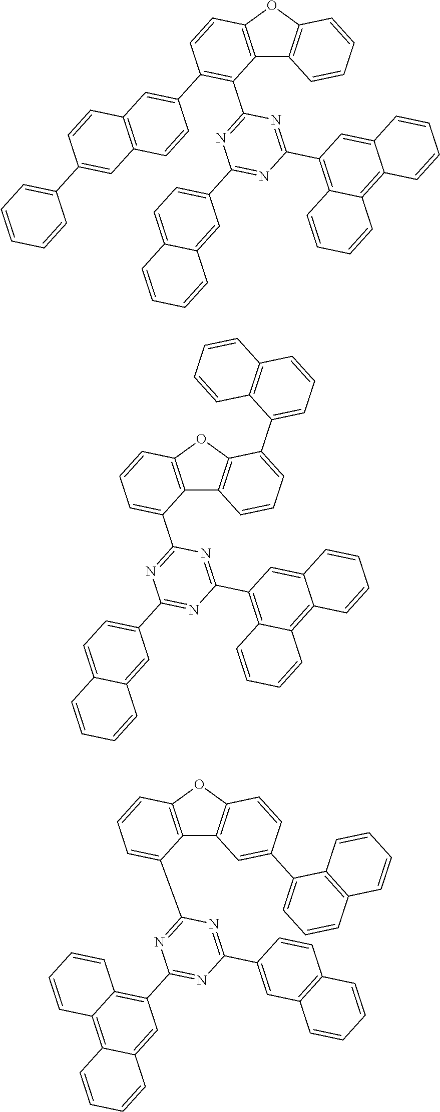

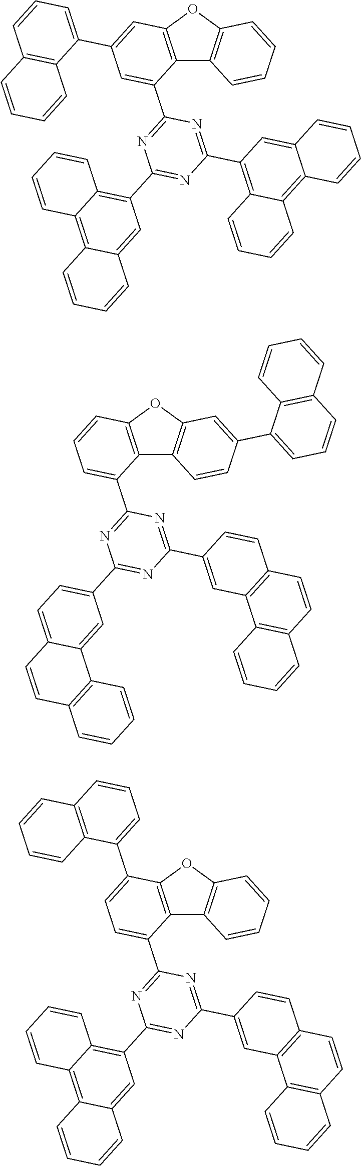

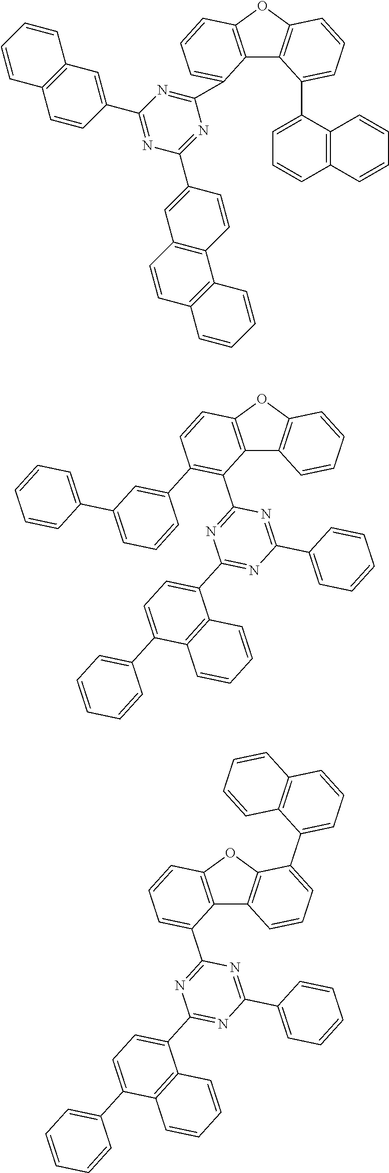 [SMILES: c1ccc2cc(-c3nc(-c4ccc5c(ccc6ccccc65)c4)nc(-c4cccc5oc6cccc(-c7cccc8ccccc78)c6c45)n3)ccc2c1 ; c1ccc(-c2cccc(-c3ccc4oc5ccccc5c4c3-c3nc(-c4ccccc4)nc(-c4ccc(-c5ccccc5)c5ccccc45)n3)c2)cc1 ; c1ccc(-c2nc(-c3ccc(-c4ccccc4)c4ccccc34)nc(-c3cccc4oc5c(-c6cccc7ccccc67)cccc5c34)n2)cc1]

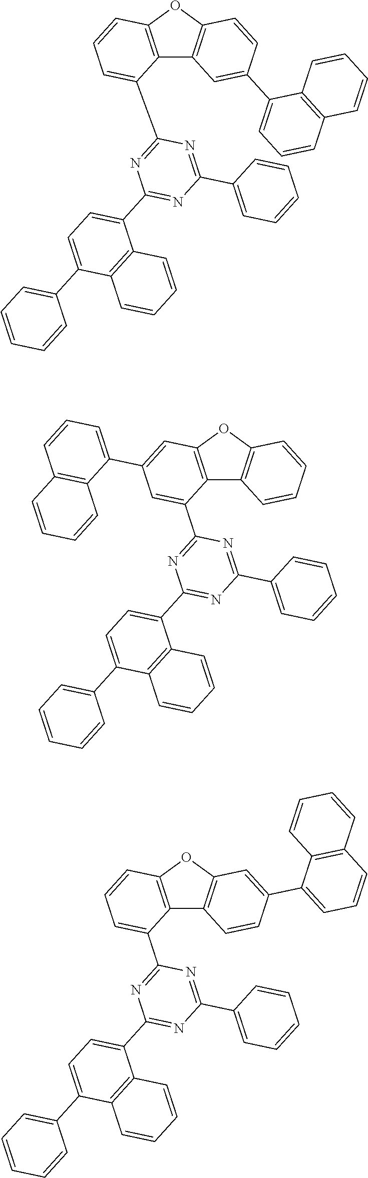 [SMILES: c1ccc(-c2nc(-c3ccc(-c4ccccc4)c4ccccc34)nc(-c3cccc4oc5ccc(-c6cccc7ccccc67)cc5c34)n2)cc1 ; c1ccc(-c2nc(-c3ccc(-c4ccccc4)c4ccccc34)nc(-c3cc(-c4cccc5ccccc45)cc4oc5ccccc5c34)n2)cc1 ; c1ccc(-c2nc(-c3ccc(-c4ccccc4)c4ccccc34)nc(-c3cccc4oc5cc(-c6cccc7ccccc67)ccc5c34)n2)cc1]

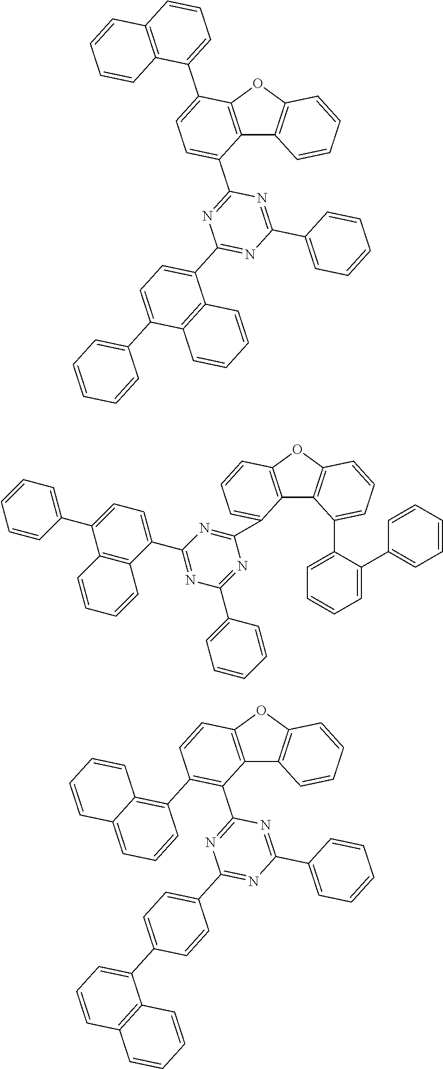

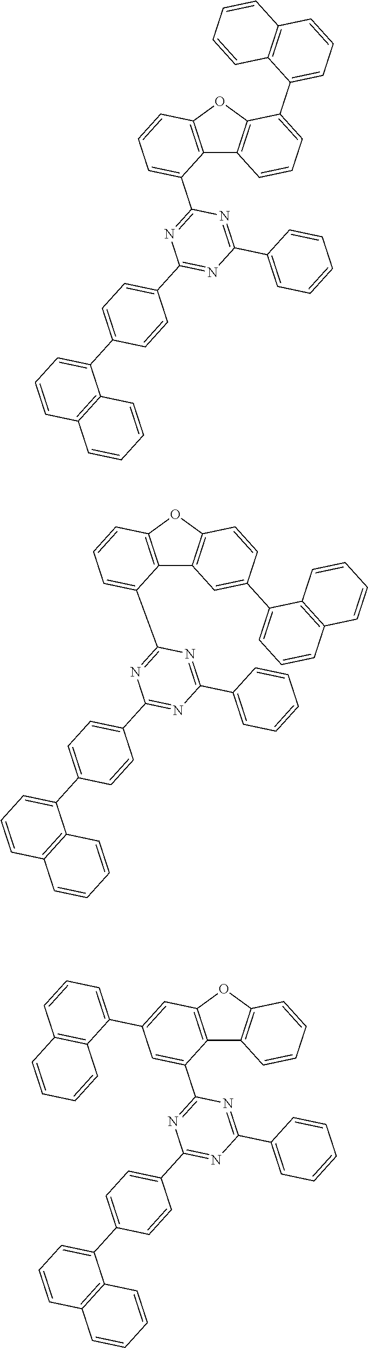 [SMILES: c1ccc(-c2nc(-c3ccc(-c4cccc5ccccc45)cc3)nc(-c3cccc4oc5c(-c6cccc7ccccc67)cccc5c34)n2)cc1 ; c1ccc(-c2nc(-c3ccc(-c4cccc5ccccc45)cc3)nc(-c3cccc4oc5ccc(-c6cccc7ccccc67)cc5c34)n2)cc1 ; c1ccc(-c2nc(-c3ccc(-c4cccc5ccccc45)cc3)nc(-c3cc(-c4cccc5ccccc45)cc4oc5ccccc5c34)n2)cc1]

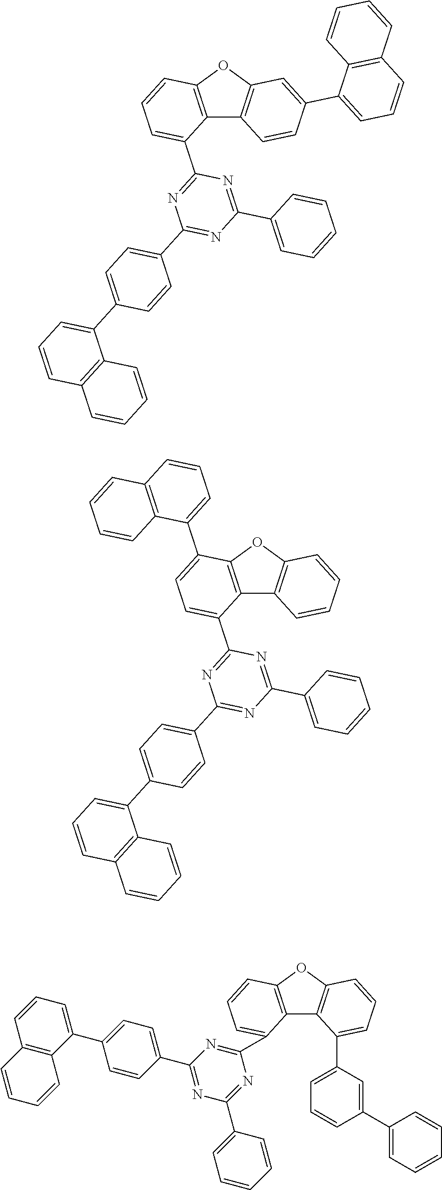 [SMILES: c1ccc(-c2nc(-c3ccc(-c4cccc5ccccc45)cc3)nc(-c3cccc4oc5cc(-c6cccc7ccccc67)ccc5c34)n2)cc1 ; c1ccc(-c2nc(-c3ccc(-c4cccc5ccccc45)cc3)nc(-c3ccc(-c4cccc5ccccc45)c4oc5ccccc5c34)n2)cc1 ; c1ccc(-c2cccc(-c3cccc4oc5cccc(-c6nc(-c7ccccc7)nc(-c7ccc(-c8cccc9ccccc89)cc7)n6)c5c34)c2)cc1]

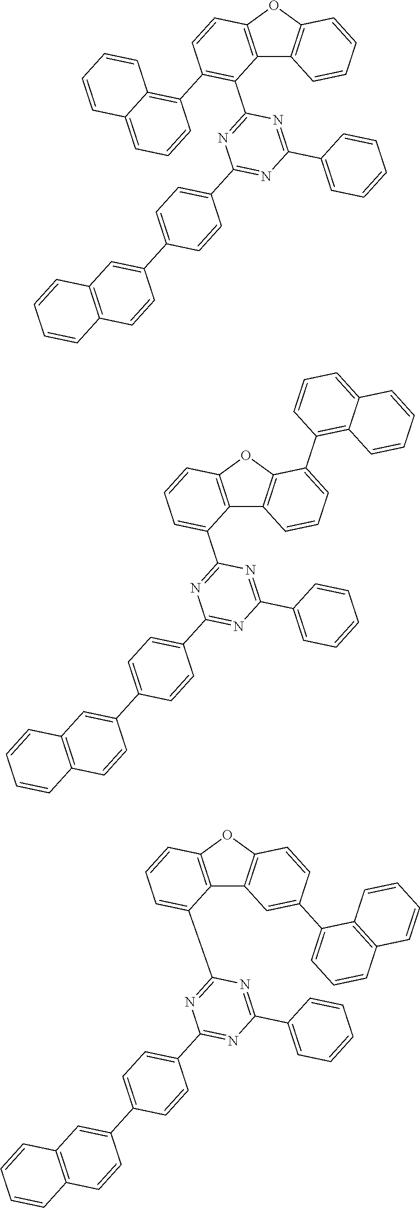 [SMILES: c1ccc(-c2nc(-c3ccc(-c4ccc5ccccc5c4)cc3)nc(-c3c(-c4cccc5ccccc45)ccc4oc5ccccc5c34)n2)cc1 ; c1ccc(-c2nc(-c3ccc(-c4ccc5ccccc5c4)cc3)nc(-c3cccc4oc5c(-c6cccc7ccccc67)cccc5c34)n2)cc1 ; c1ccc(-c2nc(-c3ccc(-c4ccc5ccccc5c4)cc3)nc(-c3cccc4oc5ccc(-c6cccc7ccccc67)cc5c34)n2)cc1]

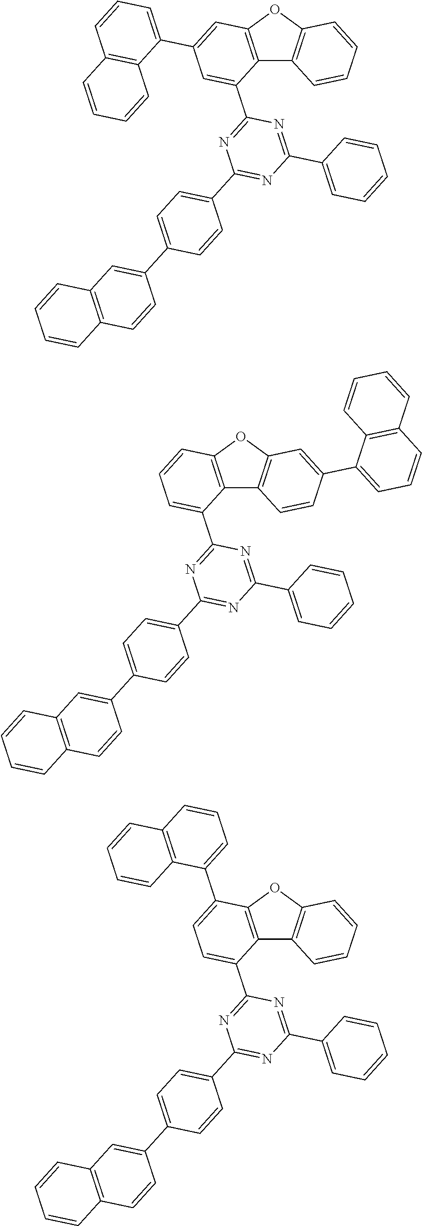 [SMILES: c1ccc(-c2nc(-c3ccc(-c4ccc5ccccc5c4)cc3)nc(-c3cc(-c4cccc5ccccc45)cc4oc5ccccc5c34)n2)cc1 ; c1ccc(-c2nc(-c3ccc(-c4ccc5ccccc5c4)cc3)nc(-c3cccc4oc5cc(-c6cccc7ccccc67)ccc5c34)n2)cc1 ; c1ccc(-c2nc(-c3ccc(-c4ccc5ccccc5c4)cc3)nc(-c3ccc(-c4cccc5ccccc45)c4oc5ccccc5c34)n2)cc1]

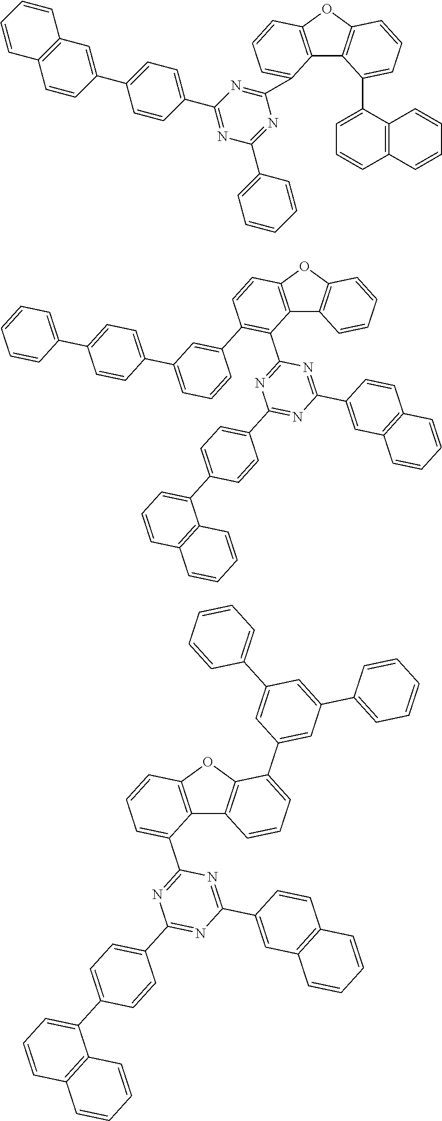

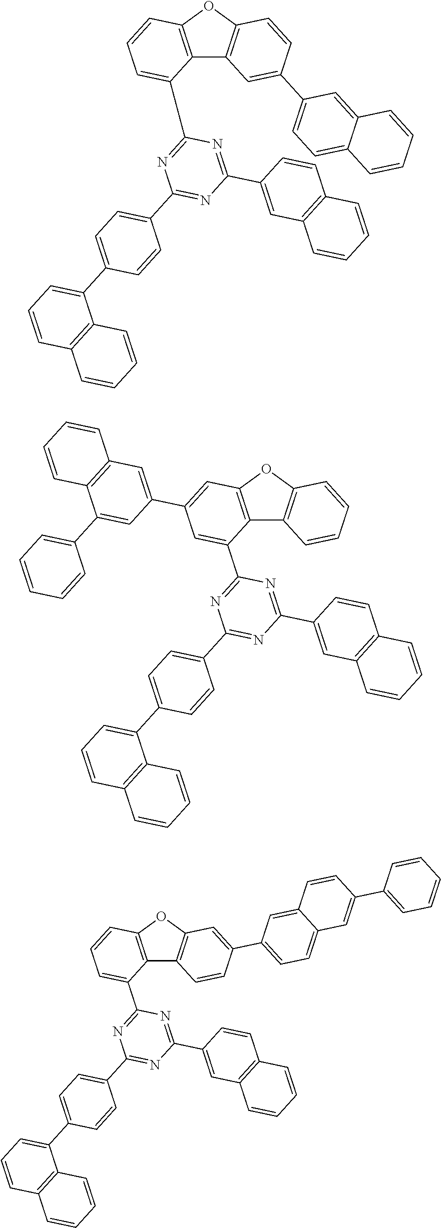

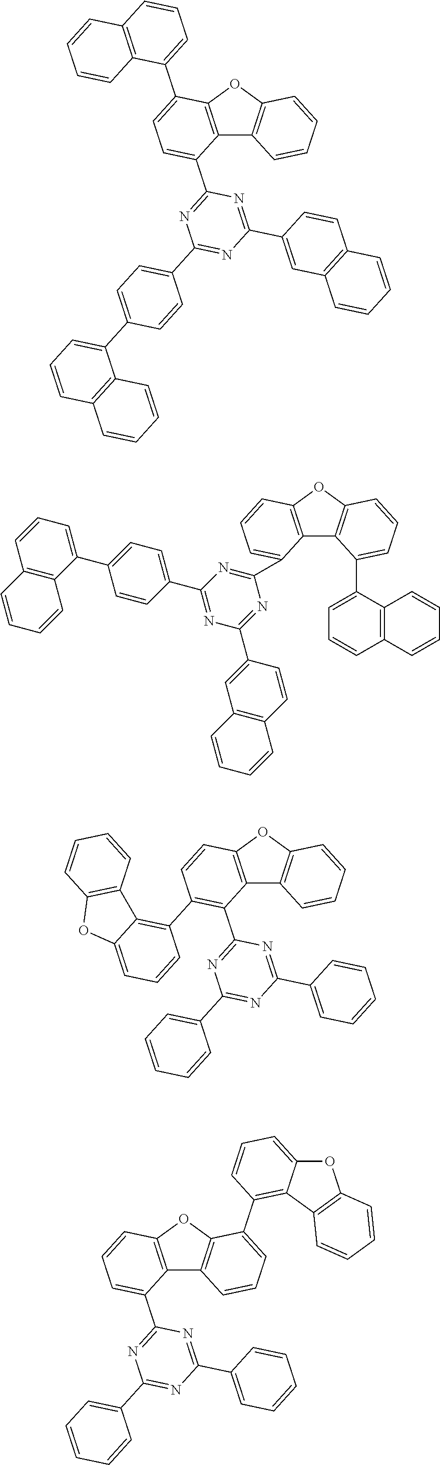 [SMILES: c1ccc2cc(-c3nc(-c4ccc(-c5cccc6ccccc56)cc4)nc(-c4ccc(-c5cccc6ccccc56)c5oc6ccccc6c45)n3)ccc2c1 ; c1ccc2cc(-c3nc(-c4ccc(-c5cccc6ccccc56)cc4)nc(-c4cccc5oc6cccc(-c7cccc8ccccc78)c6c45)n3)ccc2c1 ; c1ccc(-c2nc(-c3ccccc3)nc(-c3c(-c4cccc5oc6ccccc6c45)ccc4oc5ccccc5c34)n2)cc1 ; c1ccc(-c2nc(-c3ccccc3)nc(-c3cccc4oc5c(-c6cccc7oc8ccccc8c67)cccc5c34)n2)cc1]

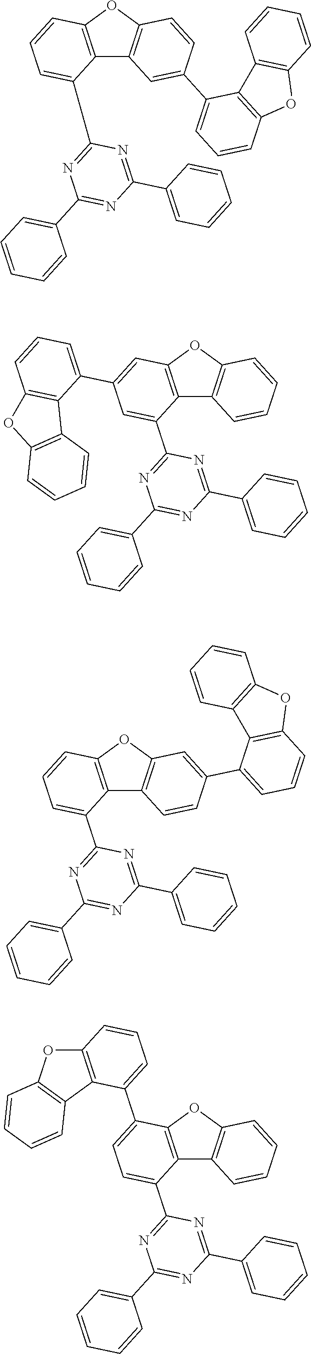 [SMILES: c1ccc(-c2nc(-c3ccccc3)nc(-c3cccc4oc5ccc(-c6cccc7oc8ccccc8c67)cc5c34)n2)cc1 ; c1ccc(-c2nc(-c3ccccc3)nc(-c3cc(-c4cccc5oc6ccccc6c45)cc4oc5ccccc5c34)n2)cc1 ; c1ccc(-c2nc(-c3ccccc3)nc(-c3cccc4oc5cc(-c6cccc7oc8ccccc8c67)ccc5c34)n2)cc1 ; c1ccc(-c2nc(-c3ccccc3)nc(-c3ccc(-c4cccc5oc6ccccc6c45)c4oc5ccccc5c34)n2)cc1]

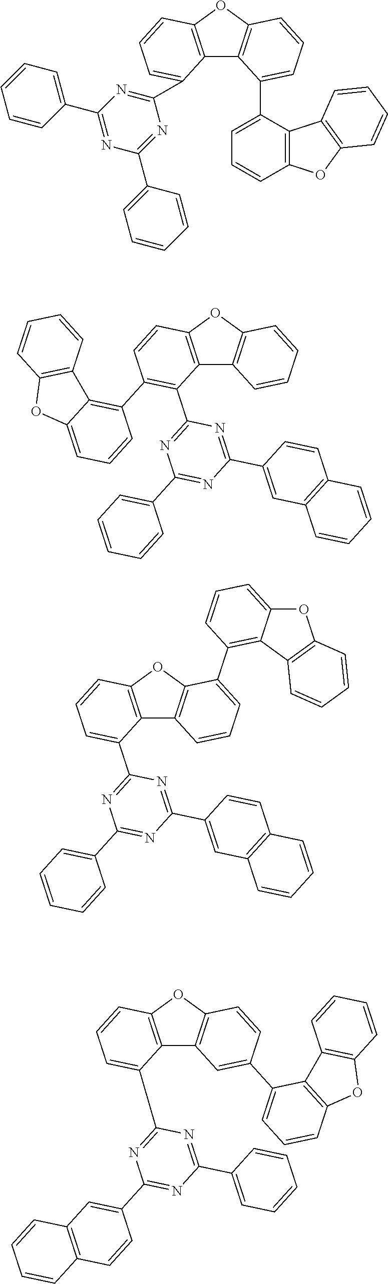 [SMILES: c1ccc(-c2nc(-c3ccccc3)nc(-c3cccc4oc5cccc(-c6cccc7oc8ccccc8c67)c5c34)n2)cc1 ; c1ccc(-c2nc(-c3ccc4ccccc4c3)nc(-c3c(-c4cccc5oc6ccccc6c45)ccc4oc5ccccc5c34)n2)cc1 ; c1ccc(-c2nc(-c3ccc4ccccc4c3)nc(-c3cccc4oc5c(-c6cccc7oc8ccccc8c67)cccc5c34)n2)cc1 ; c1ccc(-c2nc(-c3ccc4ccccc4c3)nc(-c3cccc4oc5ccc(-c6cccc7oc8ccccc8c67)cc5c34)n2)cc1]

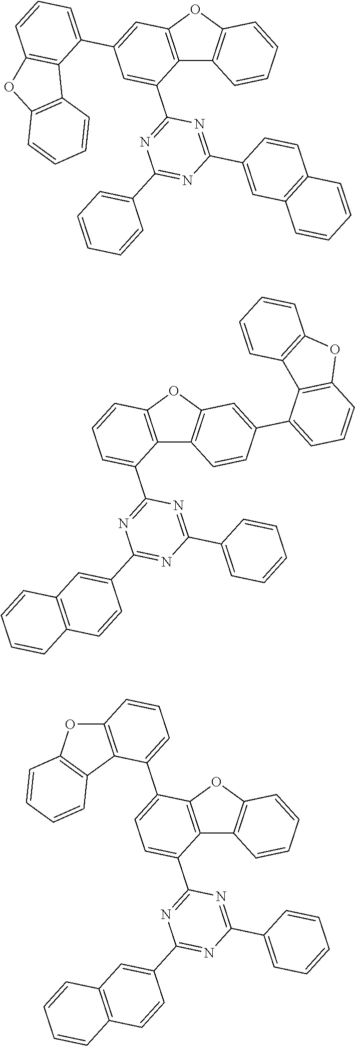 [SMILES: c1ccc(-c2nc(-c3ccc4ccccc4c3)nc(-c3cc(-c4cccc5oc6ccccc6c45)cc4oc5ccccc5c34)n2)cc1 ; c1ccc(-c2nc(-c3ccc4ccccc4c3)nc(-c3cccc4oc5cc(-c6cccc7oc8ccccc8c67)ccc5c34)n2)cc1 ; c1ccc(-c2nc(-c3ccc4ccccc4c3)nc(-c3ccc(-c4cccc5oc6ccccc6c45)c4oc5ccccc5c34)n2)cc1]

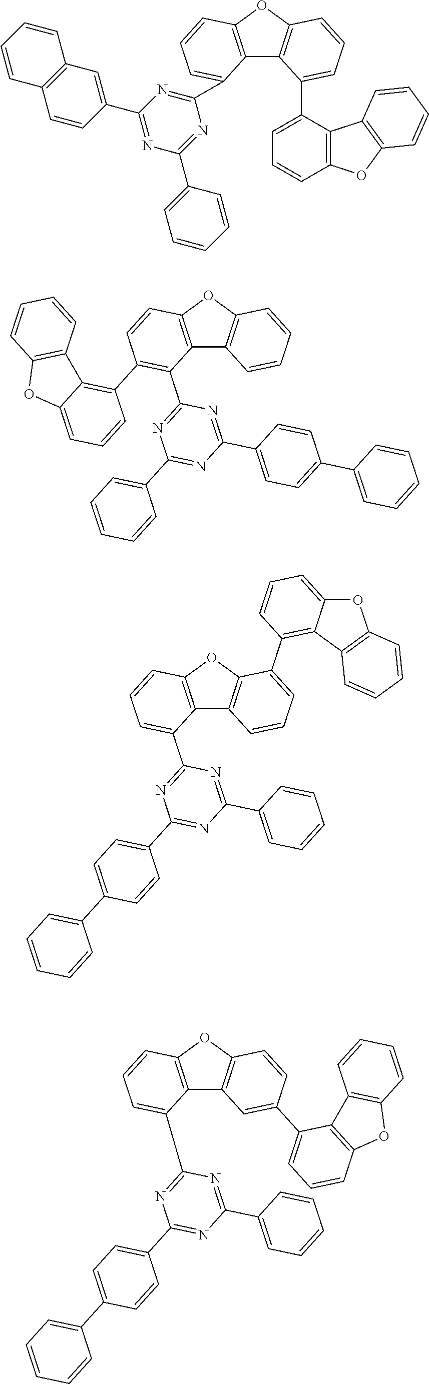

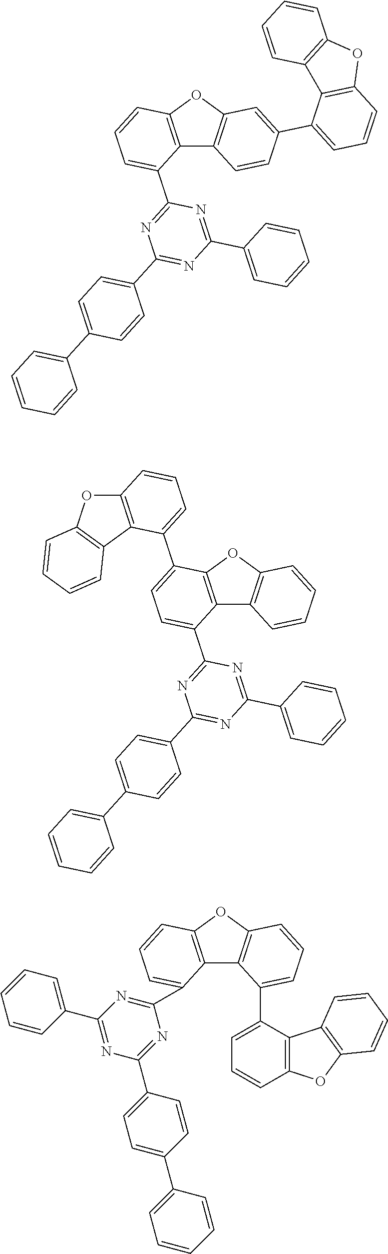 [SMILES: c1ccc(-c2ccc(-c3nc(-c4ccccc4)nc(-c4cccc5oc6cc(-c7cccc8oc9ccccc9c78)ccc6c45)n3)cc2)cc1 ; c1ccc(-c2ccc(-c3nc(-c4ccccc4)nc(-c4ccc(-c5cccc6oc7ccccc7c56)c5oc6ccccc6c45)n3)cc2)cc1 ; c1ccc(-c2ccc(-c3nc(-c4ccccc4)nc(-c4cccc5oc6cccc(-c7cccc8oc9ccccc9c78)c6c45)n3)cc2)cc1]

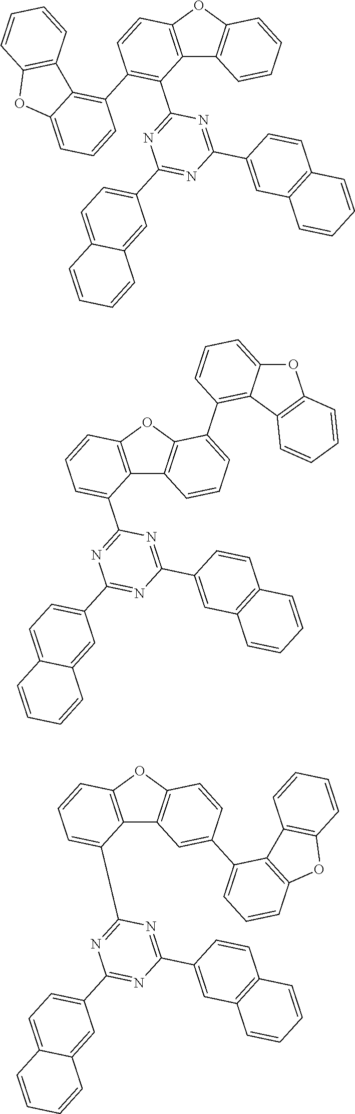 [SMILES: c1ccc2cc(-c3nc(-c4ccc5ccccc5c4)nc(-c4c(-c5cccc6oc7ccccc7c56)ccc5oc6ccccc6c45)n3)ccc2c1 ; c1ccc2cc(-c3nc(-c4ccc5ccccc5c4)nc(-c4cccc5oc6c(-c7cccc8oc9ccccc9c78)cccc6c45)n3)ccc2c1 ; c1ccc2cc(-c3nc(-c4ccc5ccccc5c4)nc(-c4cccc5oc6ccc(-c7cccc8oc9ccccc9c78)cc6c45)n3)ccc2c1]

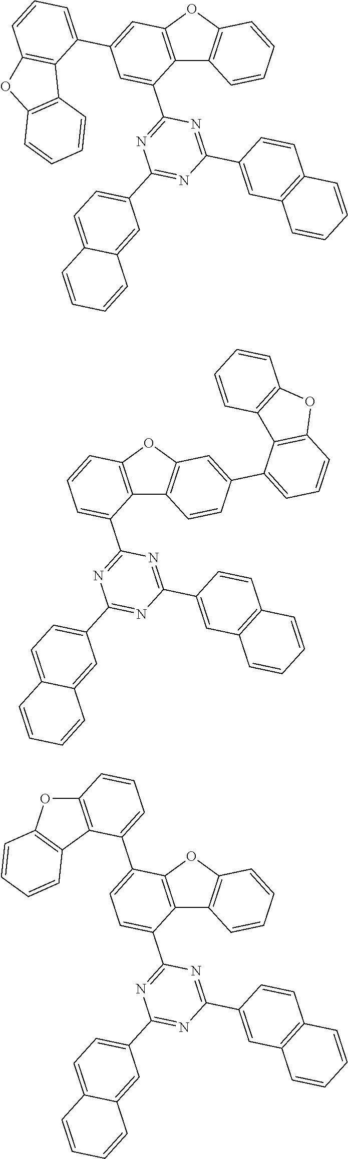 [SMILES: c1ccc2cc(-c3nc(-c4ccc5ccccc5c4)nc(-c4cc(-c5cccc6oc7ccccc7c56)cc5oc6ccccc6c45)n3)ccc2c1 ; c1ccc2cc(-c3nc(-c4ccc5ccccc5c4)nc(-c4cccc5oc6cc(-c7cccc8oc9ccccc9c78)ccc6c45)n3)ccc2c1 ; c1ccc2cc(-c3nc(-c4ccc5ccccc5c4)nc(-c4ccc(-c5cccc6oc7ccccc7c56)c5oc6ccccc6c45)n3)ccc2c1]

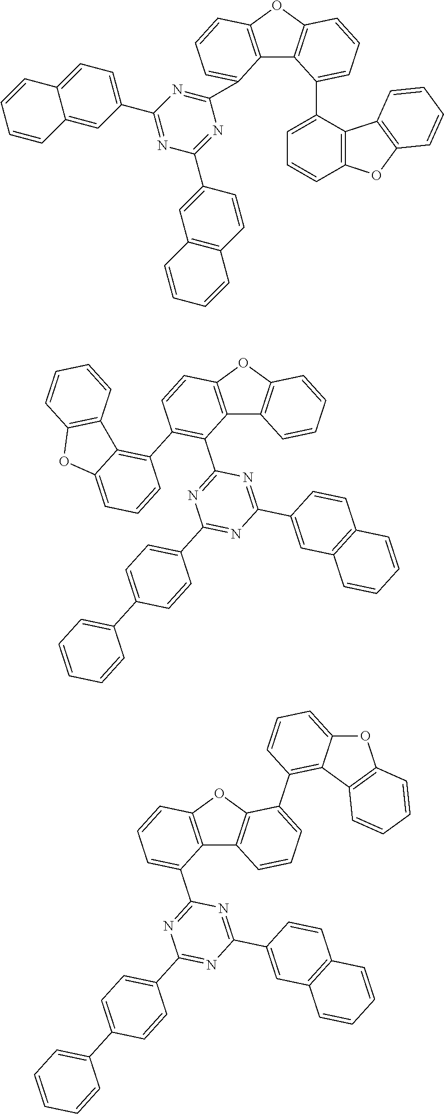 [SMILES: c1ccc2cc(-c3nc(-c4ccc5ccccc5c4)nc(-c4cccc5oc6cccc(-c7cccc8oc9ccccc9c78)c6c45)n3)ccc2c1 ; c1ccc(-c2ccc(-c3nc(-c4ccc5ccccc5c4)nc(-c4c(-c5cccc6oc7ccccc7c56)ccc5oc6ccccc6c45)n3)cc2)cc1 ; c1ccc(-c2ccc(-c3nc(-c4ccc5ccccc5c4)nc(-c4cccc5oc6c(-c7cccc8oc9ccccc9c78)cccc6c45)n3)cc2)cc1]

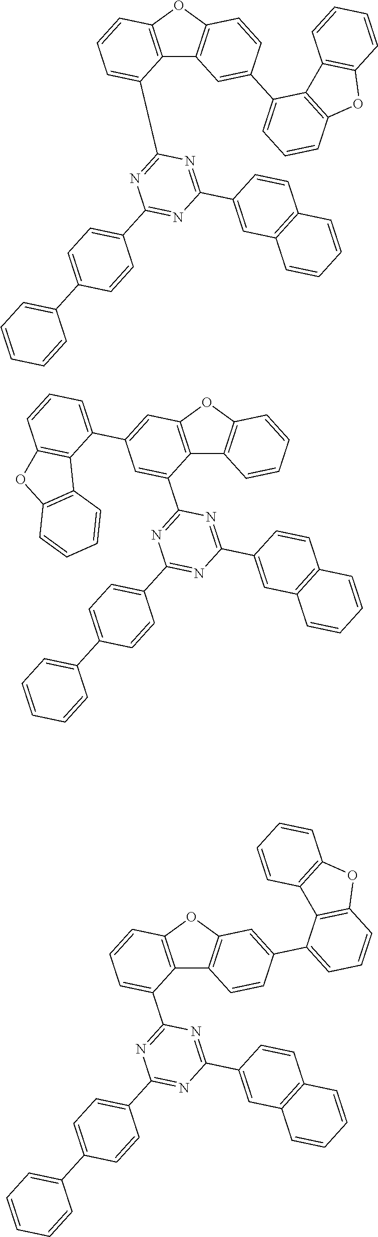

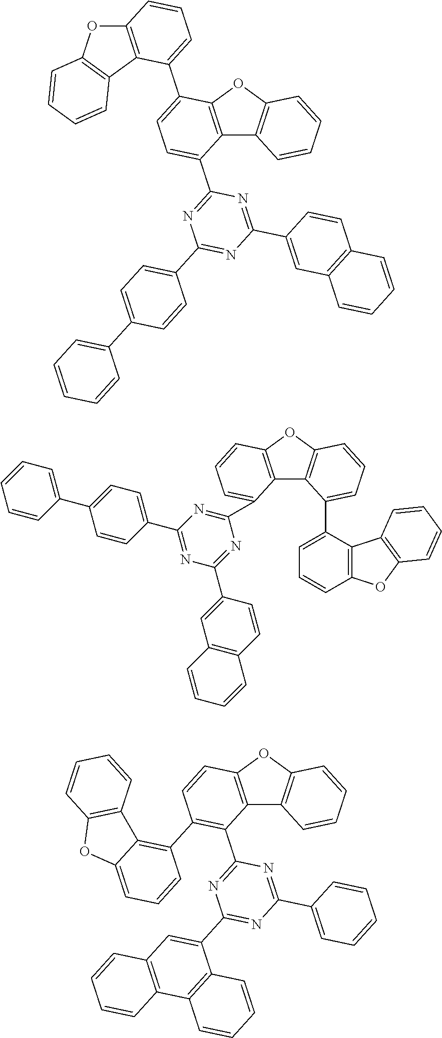 [SMILES: c1ccc(-c2ccc(-c3nc(-c4ccc5ccccc5c4)nc(-c4ccc(-c5cccc6oc7ccccc7c56)c5oc6ccccc6c45)n3)cc2)cc1 ; c1ccc(-c2ccc(-c3nc(-c4ccc5ccccc5c4)nc(-c4cccc5oc6cccc(-c7cccc8oc9ccccc9c78)c6c45)n3)cc2)cc1 ; c1ccc(-c2nc(-c3cc4ccccc4c4ccccc34)nc(-c3c(-c4cccc5oc6ccccc6c45)ccc4oc5ccccc5c34)n2)cc1]

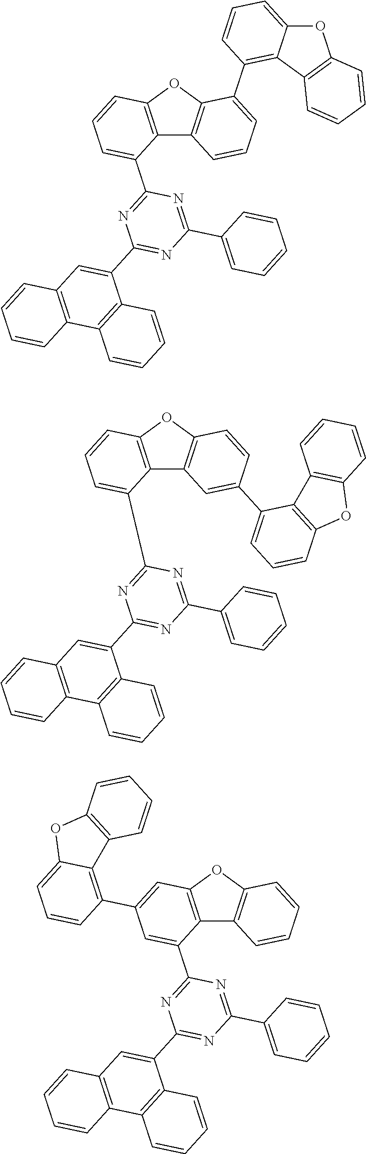

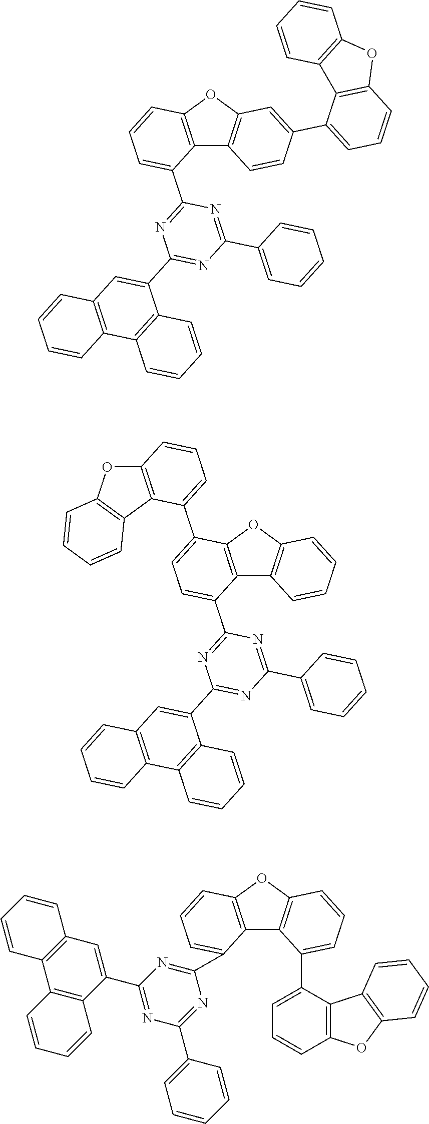 [SMILES: c1ccc(-c2nc(-c3cc4ccccc4c4ccccc34)nc(-c3cccc4oc5cc(-c6cccc7oc8ccccc8c67)ccc5c34)n2)cc1 ; c1ccc(-c2nc(-c3cc4ccccc4c4ccccc34)nc(-c3ccc(-c4cccc5oc6ccccc6c45)c4oc5ccccc5c34)n2)cc1 ; c1ccc(-c2nc(-c3cc4ccccc4c4ccccc34)nc(-c3cccc4oc5cccc(-c6cccc7oc8ccccc8c67)c5c34)n2)cc1]

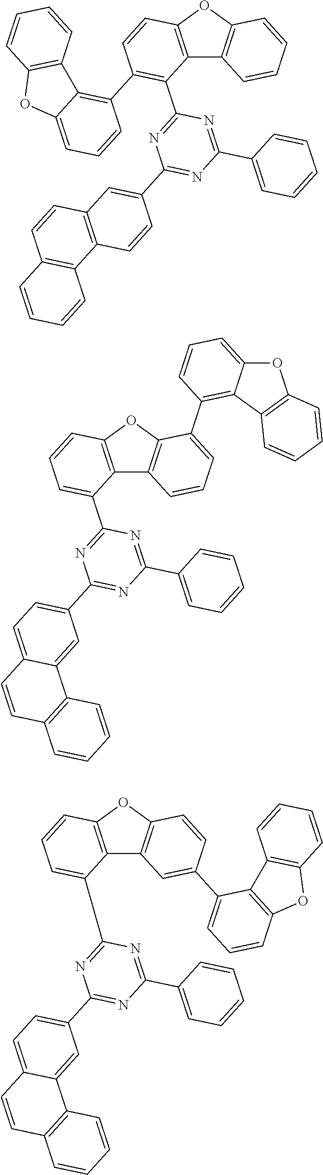 [SMILES: c1ccc(-c2nc(-c3ccc4c(ccc5ccccc54)c3)nc(-c3c(-c4cccc5oc6ccccc6c45)ccc4oc5ccccc5c34)n2)cc1 ; c1ccc(-c2nc(-c3ccc4ccc5ccccc5c4c3)nc(-c3cccc4oc5c(-c6cccc7oc8ccccc8c67)cccc5c34)n2)cc1 ; c1ccc(-c2nc(-c3ccc4ccc5ccccc5c4c3)nc(-c3cccc4oc5ccc(-c6cccc7oc8ccccc8c67)cc5c34)n2)cc1]

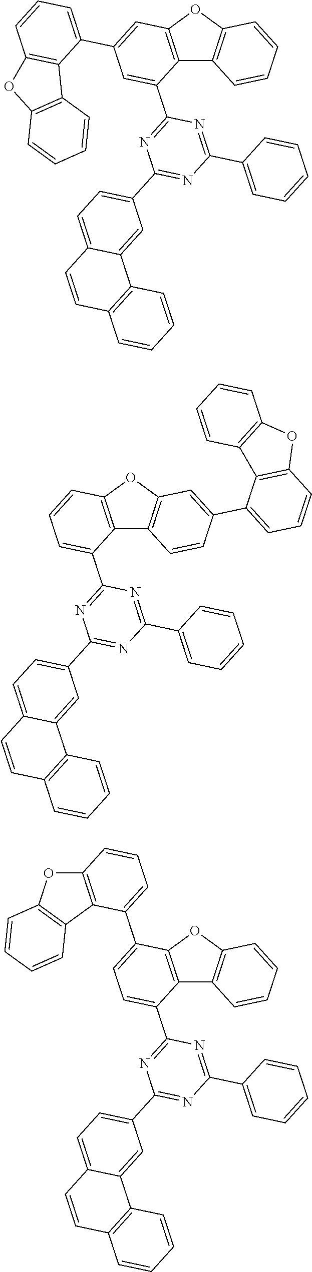

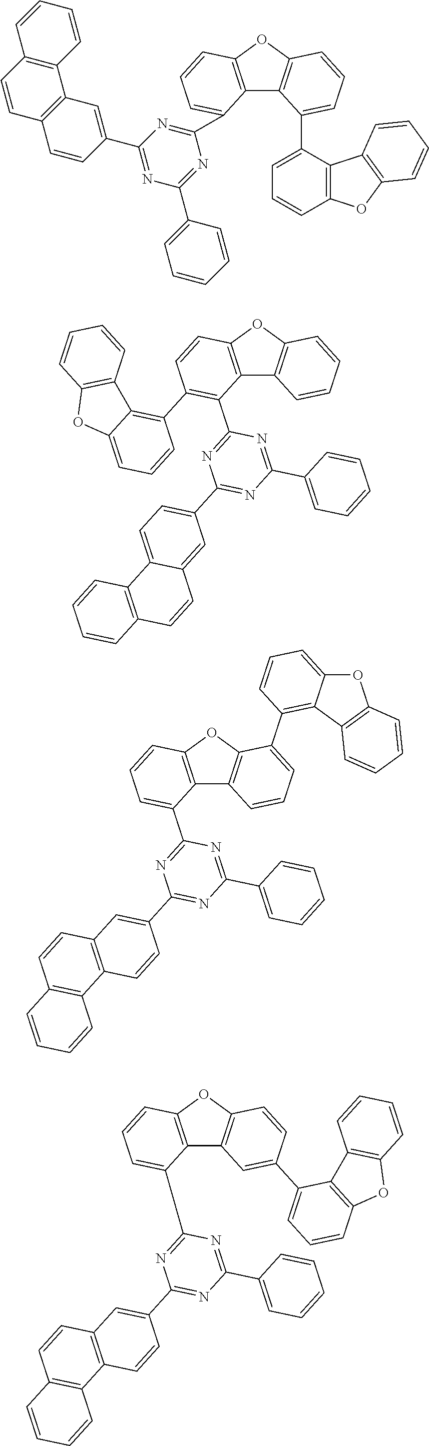 [SMILES: c1ccc(-c2nc(-c3ccc4ccc5ccccc5c4c3)nc(-c3cccc4oc5cccc(-c6cccc7oc8ccccc8c67)c5c34)n2)cc1 ; c1ccc(-c2nc(-c3ccc4c(ccc5ccccc54)c3)nc(-c3c(-c4cccc5oc6ccccc6c45)ccc4oc5ccccc5c34)n2)cc1 ; c1ccc(-c2nc(-c3ccc4c(ccc5ccccc54)c3)nc(-c3cccc4oc5c(-c6cccc7oc8ccccc8c67)cccc5c34)n2)cc1 ; c1ccc(-c2nc(-c3ccc4c(ccc5ccccc54)c3)nc(-c3cccc4oc5ccc(-c6cccc7oc8ccccc8c67)cc5c34)n2)cc1]

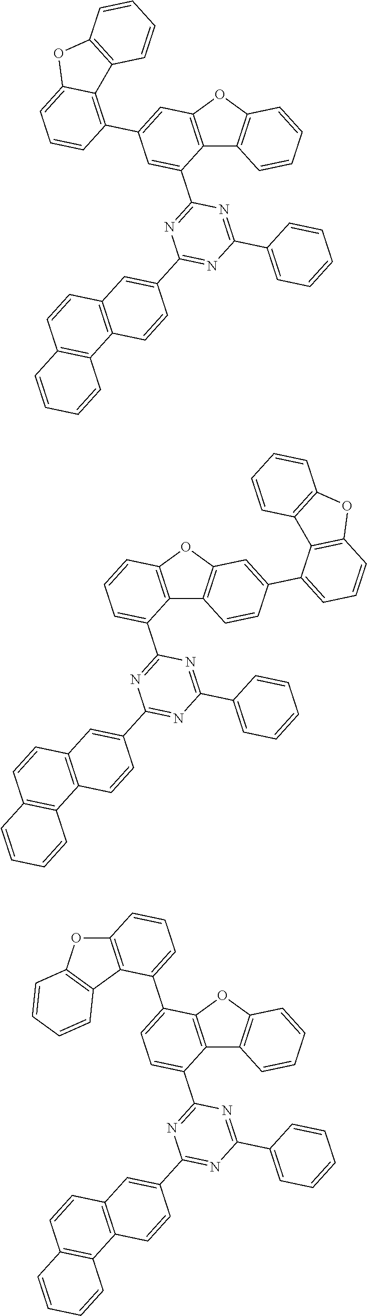 [SMILES: c1ccc(-c2nc(-c3ccc4c(ccc5ccccc54)c3)nc(-c3cc(-c4cccc5oc6ccccc6c45)cc4oc5ccccc5c34)n2)cc1 ; c1ccc(-c2nc(-c3ccc4c(ccc5ccccc54)c3)nc(-c3cccc4oc5cc(-c6cccc7oc8ccccc8c67)ccc5c34)n2)cc1 ; c1ccc(-c2nc(-c3ccc4c(ccc5ccccc54)c3)nc(-c3ccc(-c4cccc5oc6ccccc6c45)c4oc5ccccc5c34)n2)cc1]

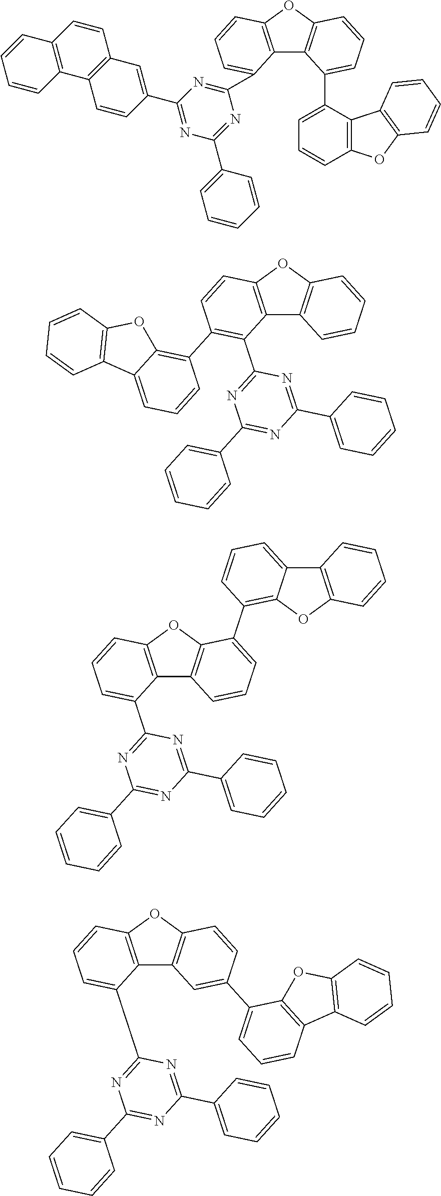 [SMILES: c1ccc(-c2nc(-c3ccc4c(ccc5ccccc54)c3)nc(-c3cccc4oc5cccc(-c6cccc7oc8ccccc8c67)c5c34)n2)cc1 ; c1ccc(-c2nc(-c3ccccc3)nc(-c3c(-c4cccc5c4oc4ccccc45)ccc4oc5ccccc5c34)n2)cc1 ; c1ccc(-c2nc(-c3ccccc3)nc(-c3cccc4oc5c(-c6cccc7c6oc6ccccc67)cccc5c34)n2)cc1 ; c1ccc(-c2nc(-c3ccccc3)nc(-c3cccc4oc5ccc(-c6cccc7c6oc6ccccc67)cc5c34)n2)cc1]

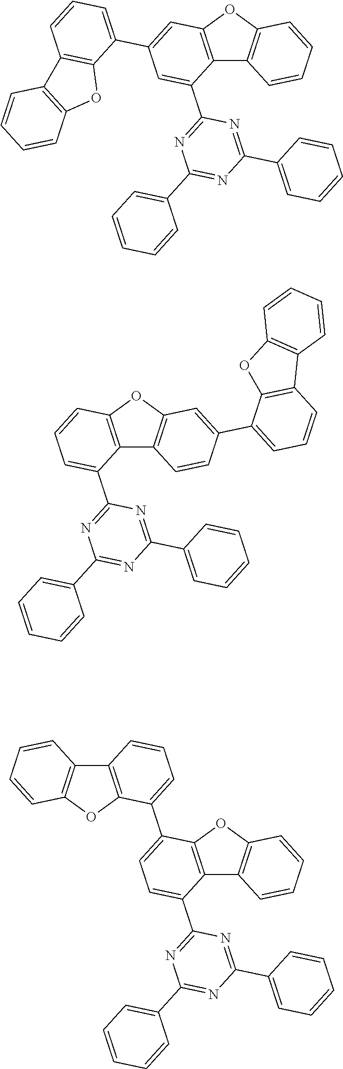 [SMILES: c1ccc(-c2nc(-c3ccccc3)nc(-c3cc(-c4cccc5c4oc4ccccc45)cc4oc5ccccc5c34)n2)cc1 ; c1ccc(-c2nc(-c3ccccc3)nc(-c3cccc4oc5cc(-c6cccc7c6oc6ccccc67)ccc5c34)n2)cc1 ; c1ccc(-c2nc(-c3ccccc3)nc(-c3ccc(-c4cccc5c4oc4ccccc45)c4oc5ccccc5c34)n2)cc1]

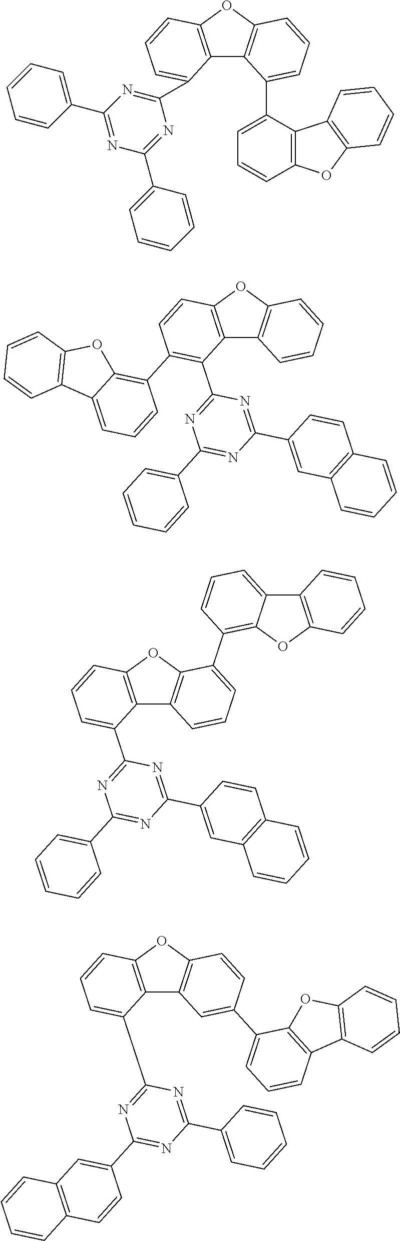 [SMILES: c1ccc(-c2nc(-c3ccccc3)nc(-c3cccc4oc5cccc(-c6cccc7oc8ccccc8c67)c5c34)n2)cc1 ; c1ccc(-c2nc(-c3ccc4ccccc4c3)nc(-c3c(-c4cccc5c4oc4ccccc45)ccc4oc5ccccc5c34)n2)cc1 ; c1ccc(-c2nc(-c3ccc4ccccc4c3)nc(-c3cccc4oc5c(-c6cccc7c6oc6ccccc67)cccc5c34)n2)cc1 ; c1ccc(-c2nc(-c3ccc4ccccc4c3)nc(-c3cccc4oc5ccc(-c6cccc7c6oc6ccccc67)cc5c34)n2)cc1]

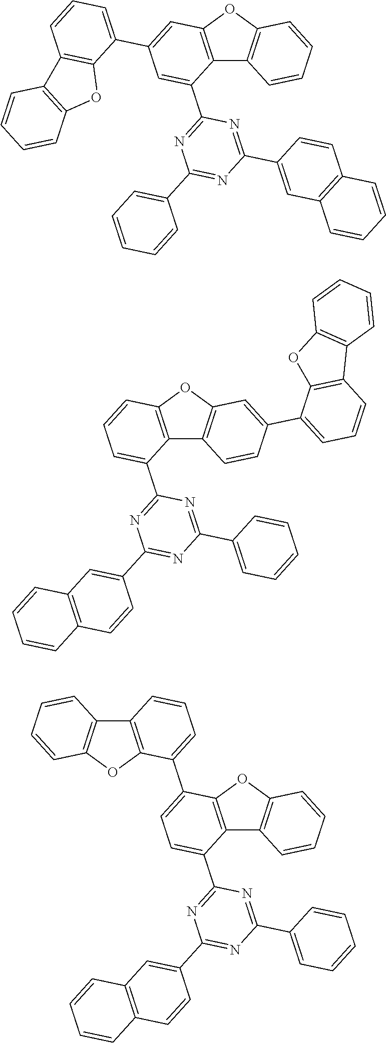

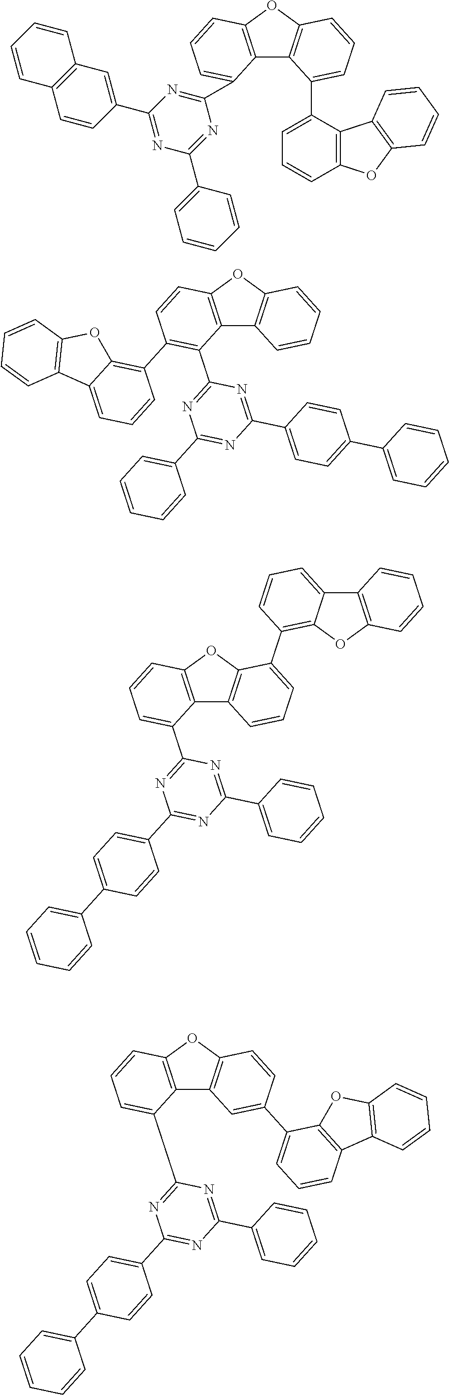

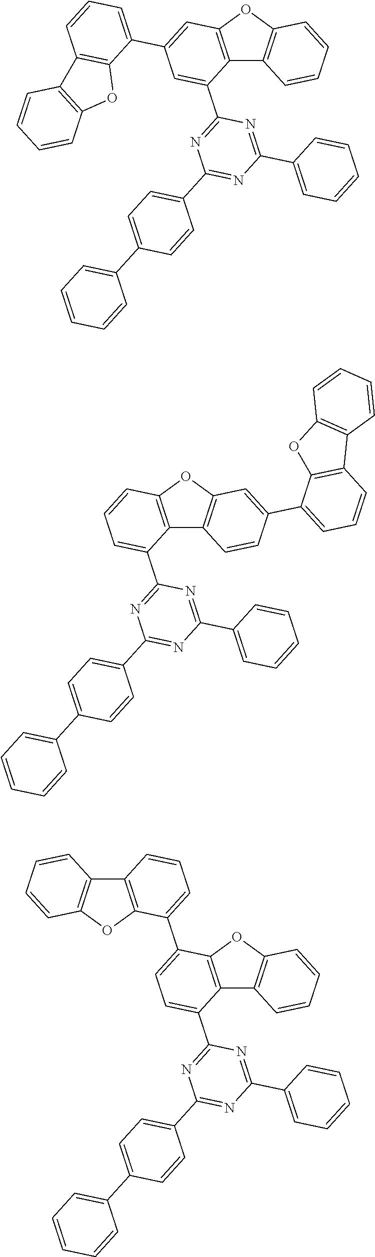 [SMILES: c1ccc(-c2ccc(-c3nc(-c4ccccc4)nc(-c4cc(-c5cccc6c5oc5ccccc56)cc5oc6ccccc6c45)n3)cc2)cc1 ; c1ccc(-c2ccc(-c3nc(-c4ccccc4)nc(-c4cccc5oc6cc(-c7cccc8c7oc7ccccc78)ccc6c45)n3)cc2)cc1 ; c1ccc(-c2ccc(-c3nc(-c4ccccc4)nc(-c4ccc(-c5cccc6c5oc5ccccc56)c5oc6ccccc6c45)n3)cc2)cc1]

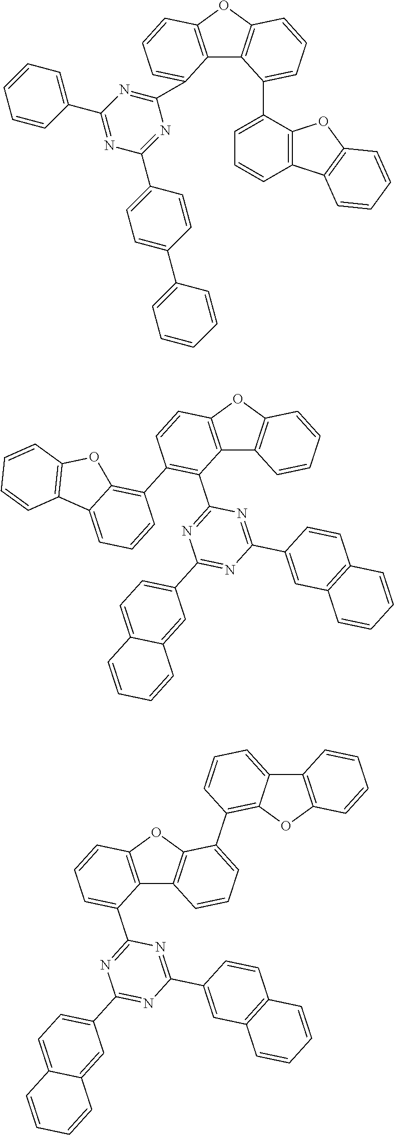 [SMILES: c1ccc(-c2ccc(-c3nc(-c4ccccc4)nc(-c4cccc5oc6cccc(-c7cccc8c7oc7ccccc78)c6c45)n3)cc2)cc1 ; c1ccc2cc(-c3nc(-c4ccc5ccccc5c4)nc(-c4c(-c5cccc6c5oc5ccccc56)ccc5oc6ccccc6c45)n3)ccc2c1 ; c1ccc2cc(-c3nc(-c4ccc5ccccc5c4)nc(-c4cccc5oc6c(-c7cccc8c7oc7ccccc78)cccc6c45)n3)ccc2c1]

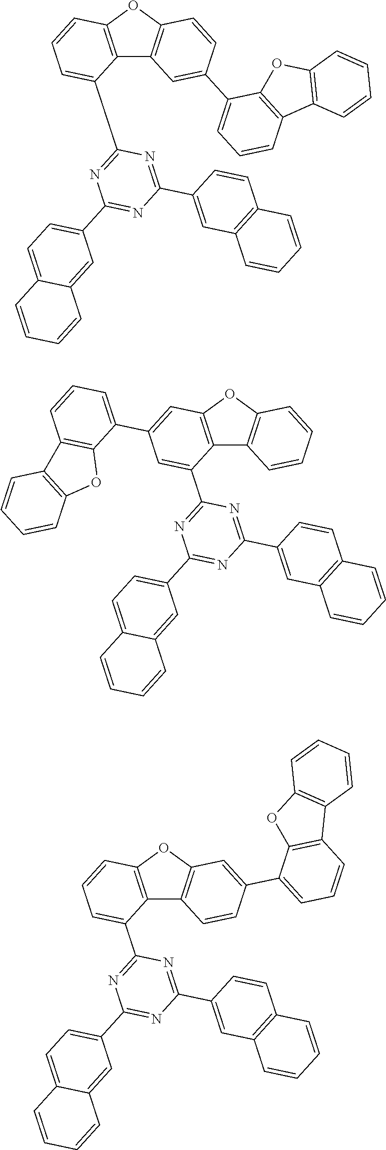

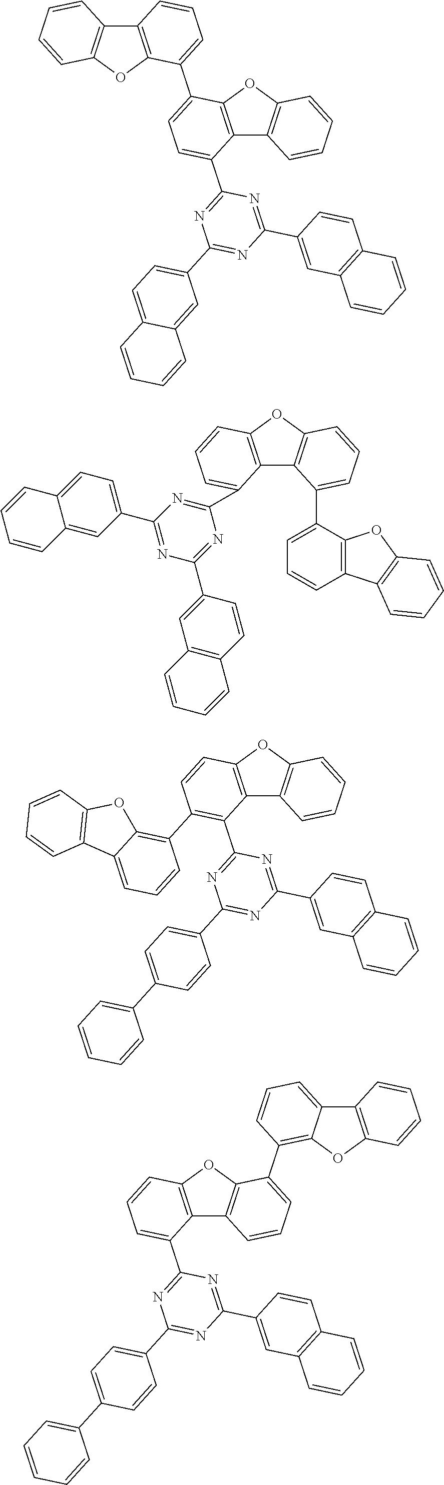

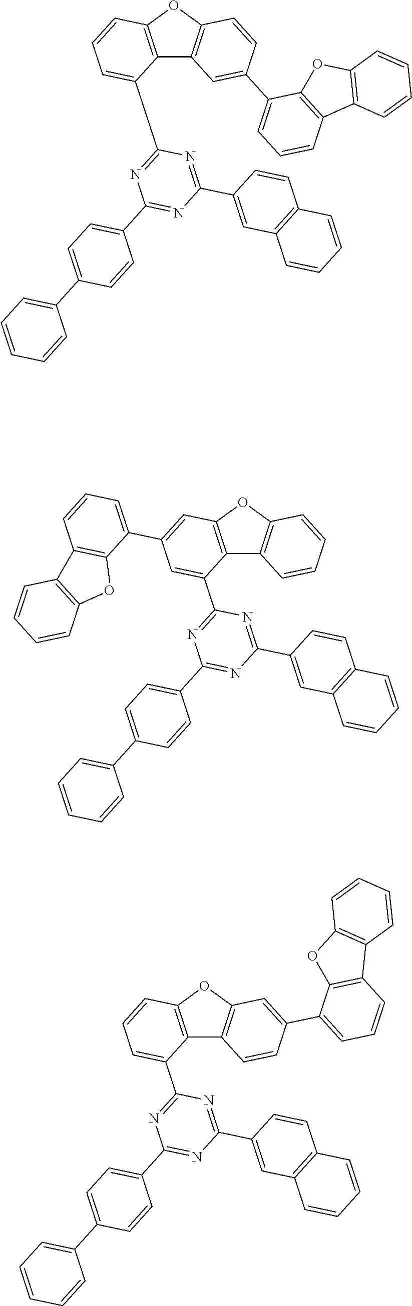 [SMILES: c1ccc(-c2ccc(-c3nc(-c4ccc5ccccc5c4)nc(-c4cccc5oc6ccc(-c7cccc8c7oc7ccccc78)cc6c45)n3)cc2)cc1 ; c1ccc(-c2ccc(-c3nc(-c4ccc5ccccc5c4)nc(-c4cc(-c5cccc6c5oc5ccccc56)cc5oc6ccccc6c45)n3)cc2)cc1 ; c1ccc(-c2ccc(-c3nc(-c4ccc5ccccc5c4)nc(-c4cccc5oc6cc(-c7cccc8c7oc7ccccc78)ccc6c45)n3)cc2)cc1]

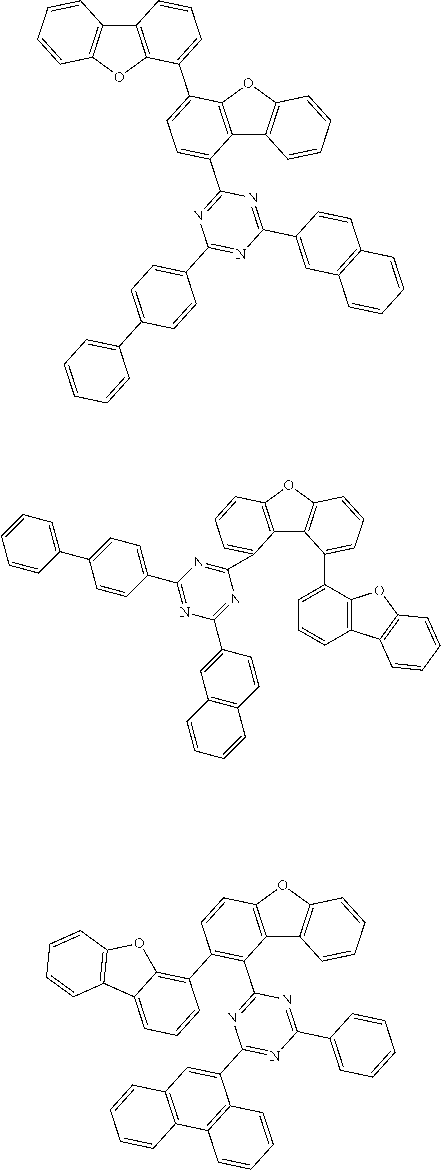 [SMILES: c1ccc(-c2ccc(-c3nc(-c4ccc5ccccc5c4)nc(-c4ccc(-c5cccc6c5oc5ccccc56)c5oc6ccccc6c45)n3)cc2)cc1 ; c1ccc(-c2ccc(-c3nc(-c4ccc5ccccc5c4)nc(-c4cccc5oc6cccc(-c7cccc8c7oc7ccccc78)c6c45)n3)cc2)cc1 ; c1ccc(-c2nc(-c3cc4ccccc4c4ccccc34)nc(-c3c(-c4cccc5c4oc4ccccc45)ccc4oc5ccccc5c34)n2)cc1]

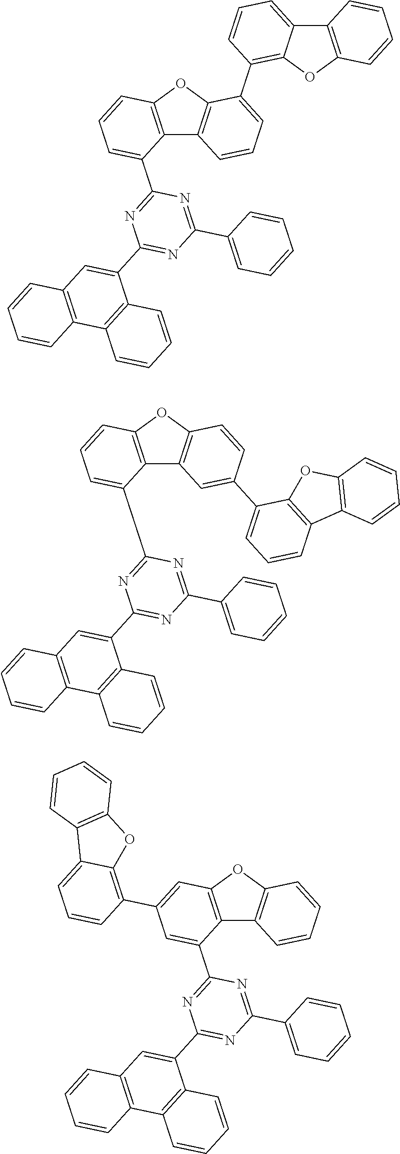

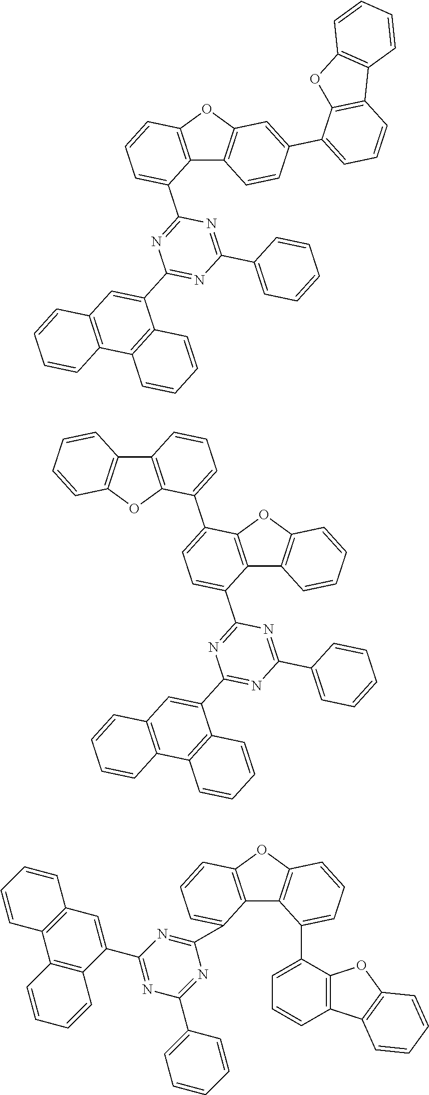

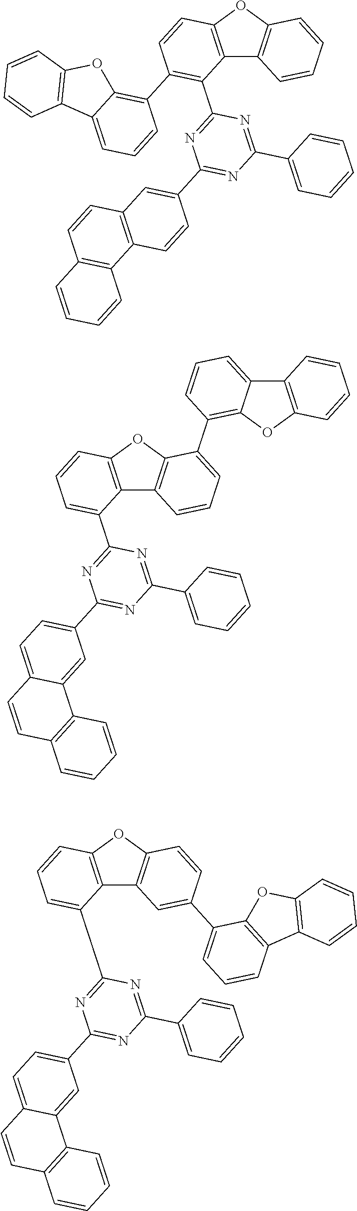 [SMILES: c1ccc(-c2nc(-c3ccc4c(ccc5ccccc54)c3)nc(-c3c(-c4cccc5c4oc4ccccc45)ccc4oc5ccccc5c34)n2)cc1 ; c1ccc(-c2nc(-c3ccc4ccc5ccccc5c4c3)nc(-c3cccc4oc5c(-c6cccc7c6oc6ccccc67)cccc5c34)n2)cc1 ; c1ccc(-c2nc(-c3ccc4ccc5ccccc5c4c3)nc(-c3cccc4oc5ccc(-c6cccc7c6oc6ccccc67)cc5c34)n2)cc1]

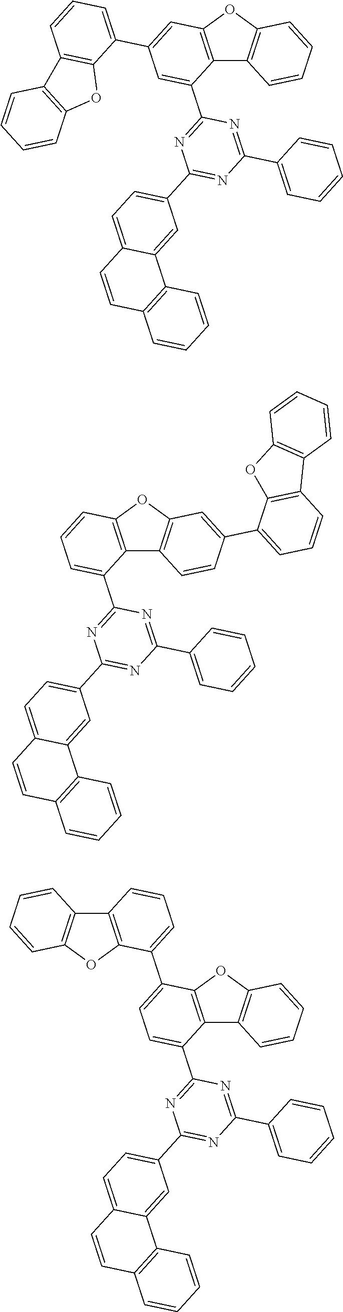 [SMILES: c1ccc(-c2nc(-c3ccc4ccc5ccccc5c4c3)nc(-c3cc(-c4cccc5c4oc4ccccc45)cc4oc5ccccc5c34)n2)cc1 ; c1ccc(-c2nc(-c3ccc4ccc5ccccc5c4c3)nc(-c3cccc4oc5cc(-c6cccc7c6oc6ccccc67)ccc5c34)n2)cc1 ; c1ccc(-c2nc(-c3ccc4ccc5ccccc5c4c3)nc(-c3ccc(-c4cccc5c4oc4ccccc45)c4oc5ccccc5c34)n2)cc1]

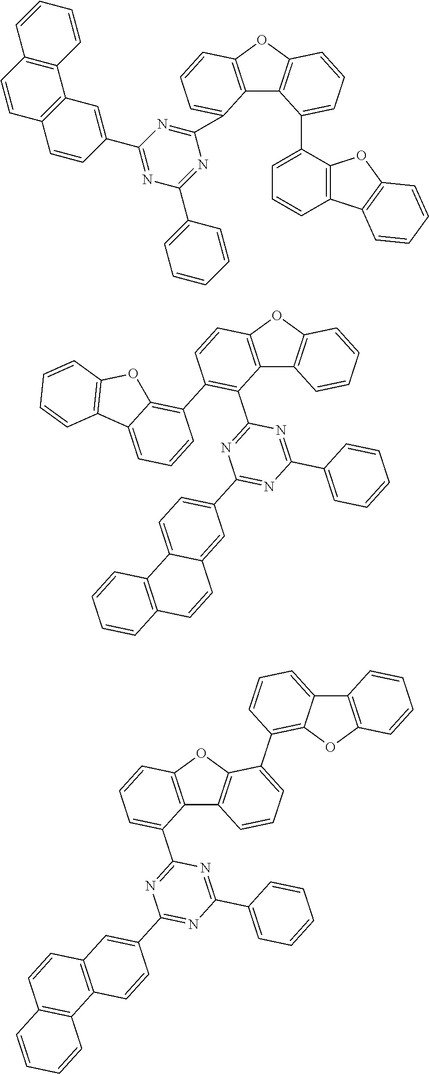

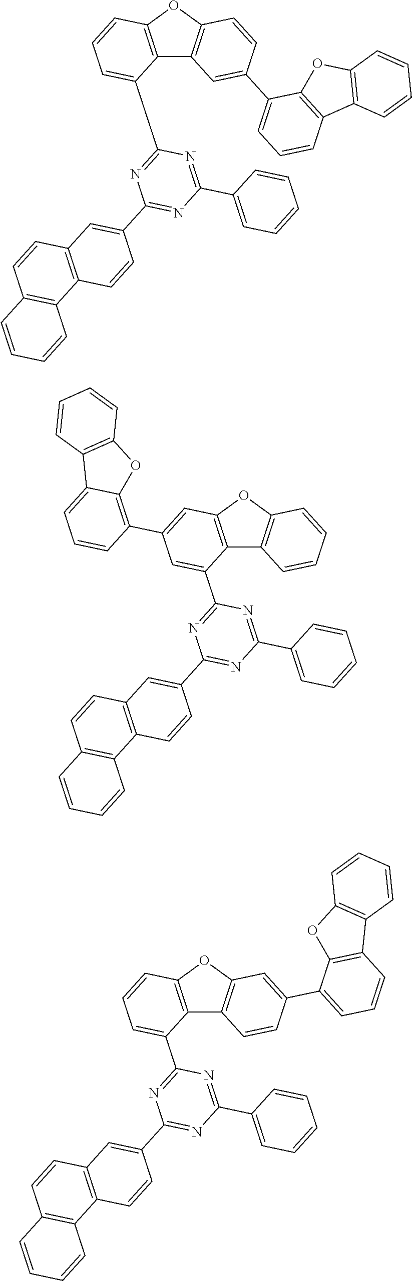

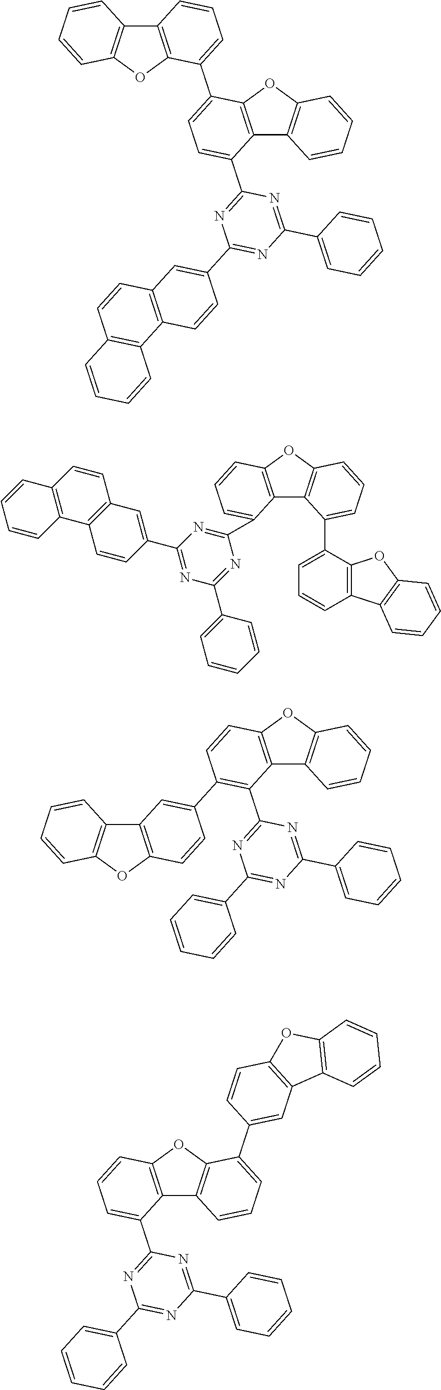 [SMILES: c1ccc(-c2nc(-c3ccc4c(ccc5ccccc54)c3)nc(-c3ccc(-c4cccc5c4oc4ccccc45)c4oc5ccccc5c34)n2)cc1 ; c1ccc(-c2nc(-c3ccc4c(ccc5ccccc54)c3)nc(-c3cccc4oc5cccc(-c6cccc7c6oc6ccccc67)c5c34)n2)cc1 ; c1ccc(-c2nc(-c3ccccc3)nc(-c3c(-c4ccc5oc6ccccc6c5c4)ccc4oc5ccccc5c34)n2)cc1 ; c1ccc(-c2nc(-c3ccccc3)nc(-c3cccc4oc5c(-c6ccc7oc8ccccc8c7c6)cccc5c34)n2)cc1]

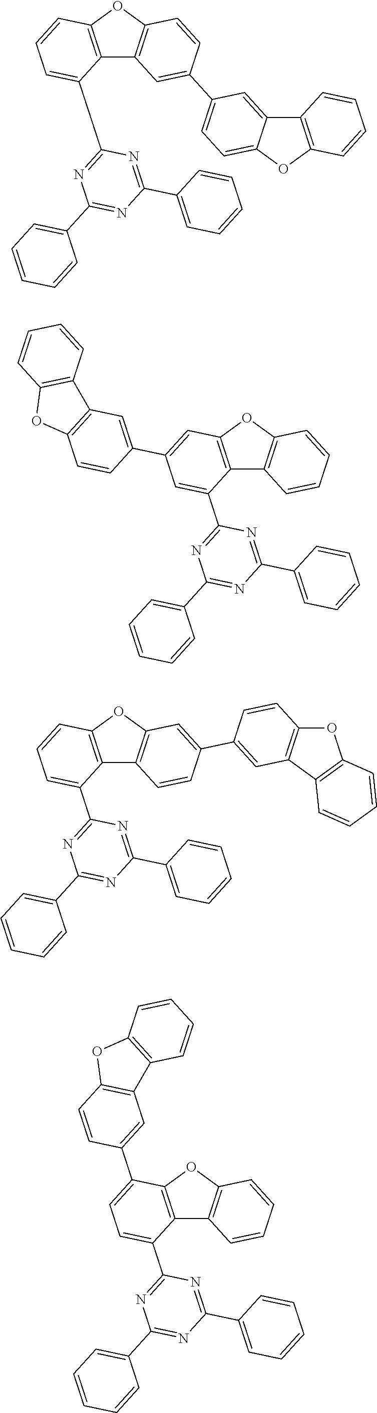

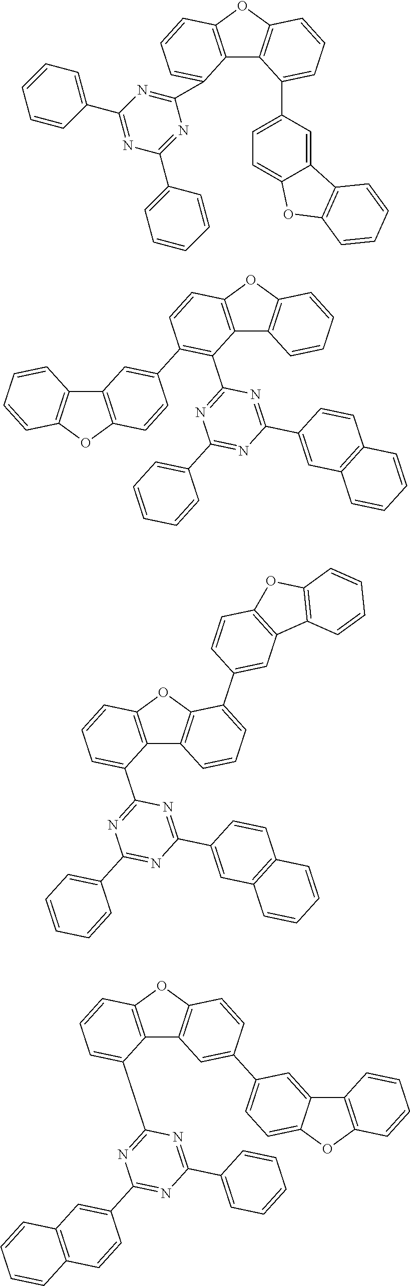

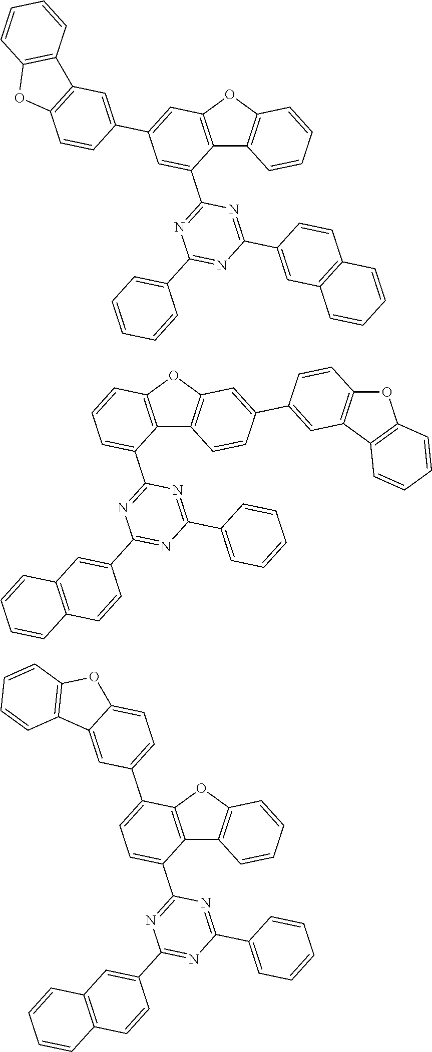

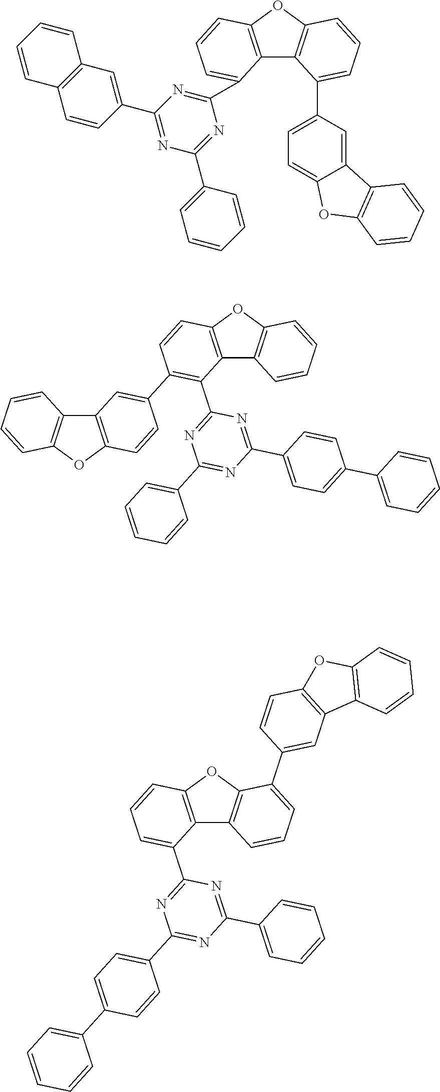 [SMILES: c1ccc(-c2nc(-c3ccc4ccccc4c3)nc(-c3cccc4oc5cccc(-c6ccc7oc8ccccc8c7c6)c5c34)n2)cc1 ; c1ccc(-c2ccc(-c3nc(-c4ccccc4)nc(-c4c(-c5ccc6oc7ccccc7c6c5)ccc5oc6ccccc6c45)n3)cc2)cc1 ; c1ccc(-c2ccc(-c3nc(-c4ccccc4)nc(-c4cccc5oc6c(-c7ccc8oc9ccccc9c8c7)cccc6c45)n3)cc2)cc1]

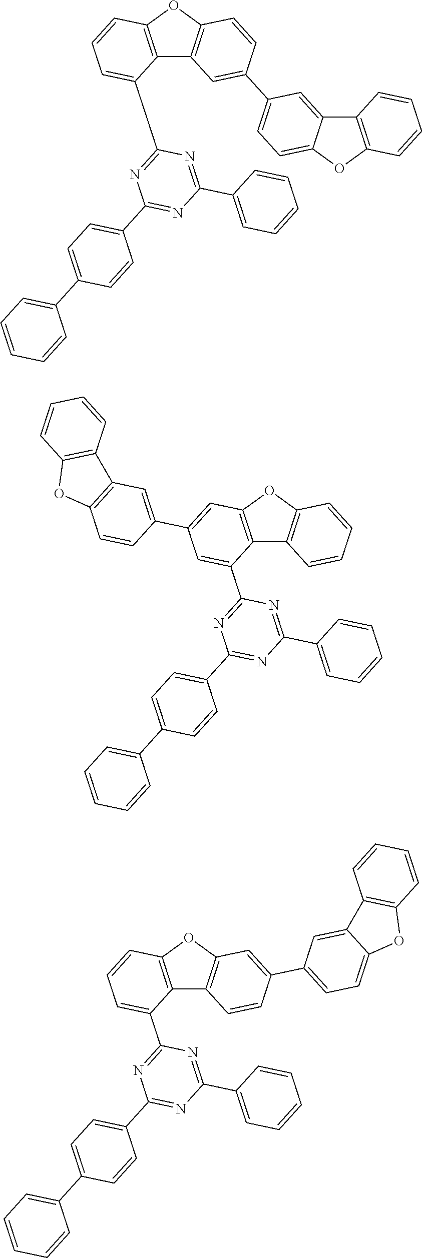

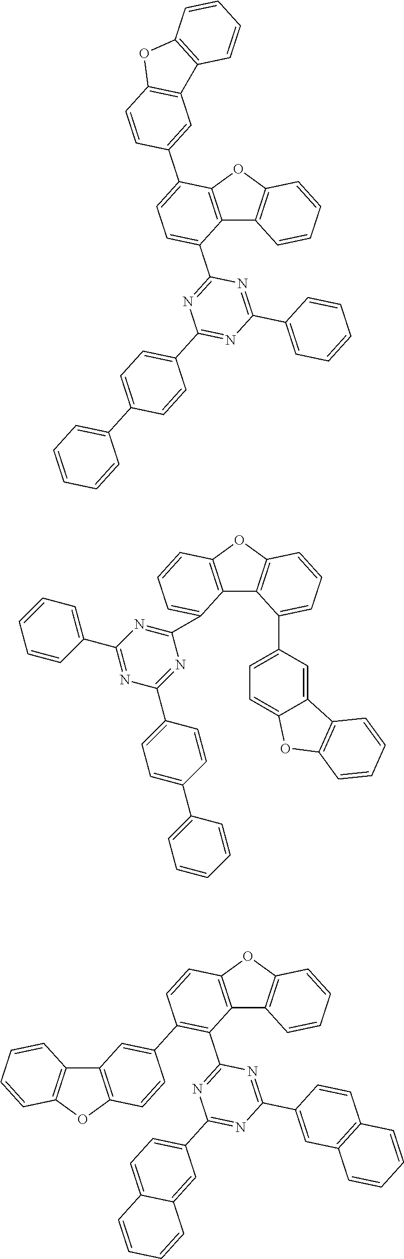 [SMILES: c1ccc(-c2ccc(-c3nc(-c4ccccc4)nc(-c4ccc(-c5ccc6oc7ccccc7c6c5)c5oc6ccccc6c45)n3)cc2)cc1 ; c1ccc(-c2ccc(-c3nc(-c4ccccc4)nc(-c4cccc5oc6cccc(-c7ccc8oc9ccccc9c8c7)c6c45)n3)cc2)cc1 ; c1ccc2cc(-c3nc(-c4ccc5ccccc5c4)nc(-c4c(-c5ccc6oc7ccccc7c6c5)ccc5oc6ccccc6c45)n3)ccc2c1]

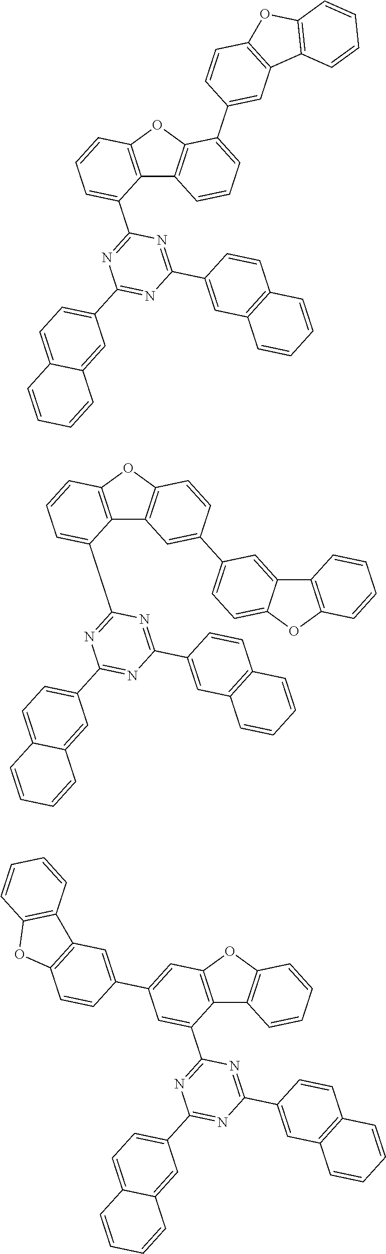 [SMILES: c1ccc2cc(-c3nc(-c4ccc5ccccc5c4)nc(-c4cccc5oc6c(-c7ccc8oc9ccccc9c8c7)cccc6c45)n3)ccc2c1 ; c1ccc2cc(-c3nc(-c4ccc5ccccc5c4)nc(-c4cccc5oc6ccc(-c7ccc8oc9ccccc9c8c7)cc6c45)n3)ccc2c1 ; c1ccc2cc(-c3nc(-c4ccc5ccccc5c4)nc(-c4cc(-c5ccc6oc7ccccc7c6c5)cc5oc6ccccc6c45)n3)ccc2c1]

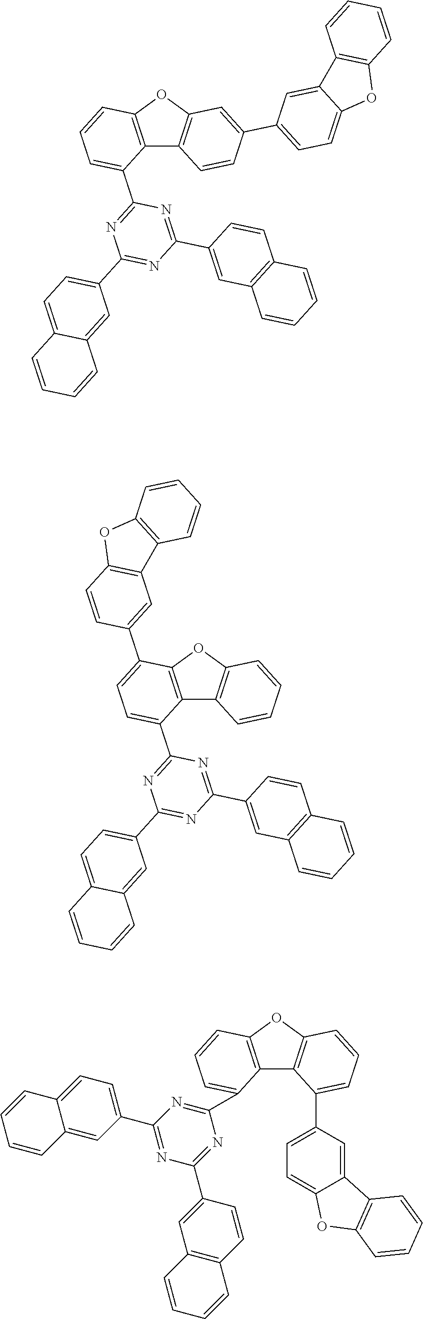 [SMILES: c1ccc2cc(-c3nc(-c4ccc5ccccc5c4)nc(-c4cccc5oc6cc(-c7ccc8oc9ccccc9c8c7)ccc6c45)n3)ccc2c1 ; c1ccc2cc(-c3nc(-c4ccc5ccccc5c4)nc(-c4ccc(-c5ccc6oc7ccccc7c6c5)c5oc6ccccc6c45)n3)ccc2c1 ; c1ccc2cc(-c3nc(-c4ccc5ccccc5c4)nc(-c4cccc5oc6cccc(-c7ccc8oc9ccccc9c8c7)c6c45)n3)ccc2c1]

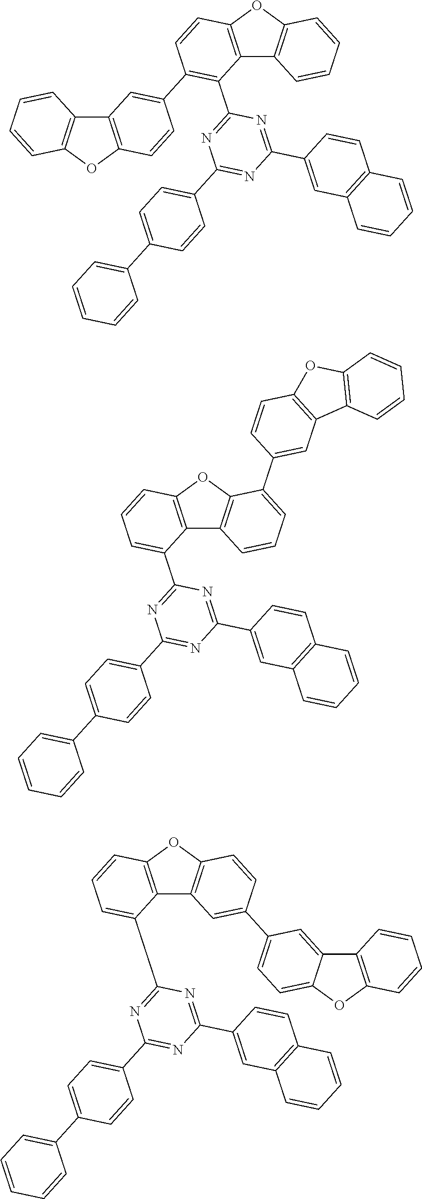 [SMILES: c1ccc(-c2ccc(-c3nc(-c4ccc5ccccc5c4)nc(-c4c(-c5ccc6oc7ccccc7c6c5)ccc5oc6ccccc6c45)n3)cc2)cc1 ; c1ccc(-c2ccc(-c3nc(-c4ccc5ccccc5c4)nc(-c4cccc5oc6c(-c7ccc8oc9ccccc9c8c7)cccc6c45)n3)cc2)cc1 ; c1ccc(-c2ccc(-c3nc(-c4ccc5ccccc5c4)nc(-c4cccc5oc6ccc(-c7ccc8oc9ccccc9c8c7)cc6c45)n3)cc2)cc1]

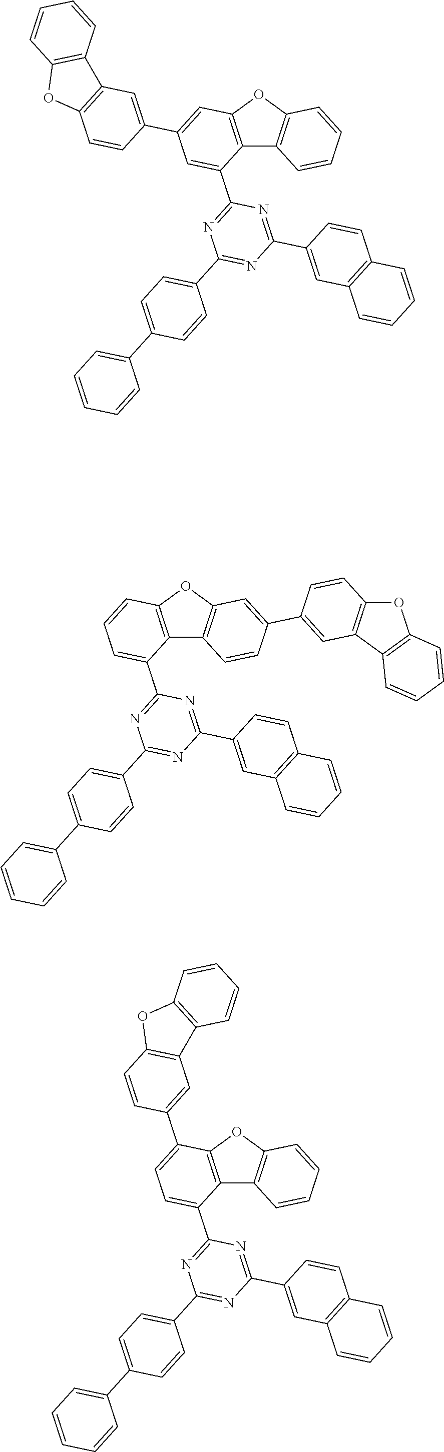 [SMILES: c1ccc(-c2ccc(-c3nc(-c4ccc5ccccc5c4)nc(-c4cc(-c5ccc6oc7ccccc7c6c5)cc5oc6ccccc6c45)n3)cc2)cc1 ; c1ccc(-c2ccc(-c3nc(-c4ccc5ccccc5c4)nc(-c4cccc5oc6cc(-c7ccc8oc9ccccc9c8c7)ccc6c45)n3)cc2)cc1 ; c1ccc(-c2ccc(-c3nc(-c4ccc5ccccc5c4)nc(-c4ccc(-c5ccc6oc7ccccc7c6c5)c5oc6ccccc6c45)n3)cc2)cc1]

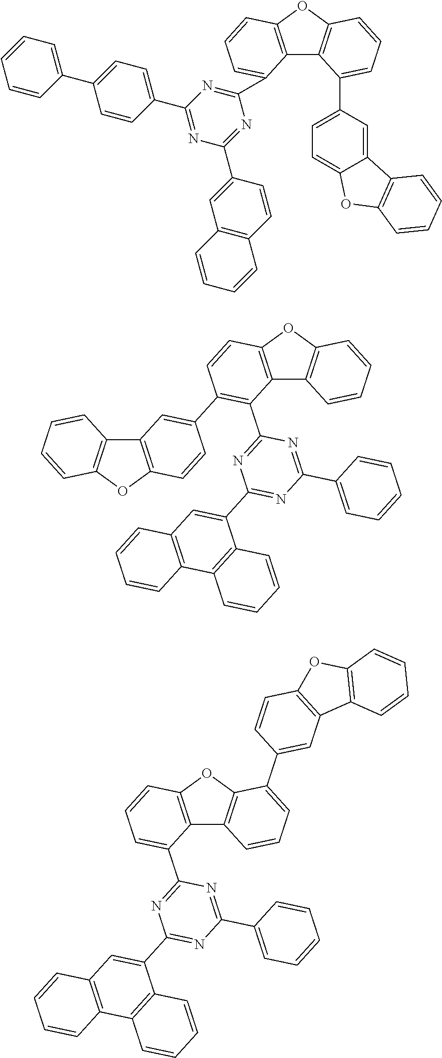 [SMILES: c1ccc(-c2ccc(-c3nc(-c4ccc5ccccc5c4)nc(-c4cccc5oc6cccc(-c7ccc8oc9ccccc9c8c7)c6c45)n3)cc2)cc1 ; c1ccc(-c2nc(-c3cc4ccccc4c4ccccc34)nc(-c3c(-c4ccc5oc6ccccc6c5c4)ccc4oc5ccccc5c34)n2)cc1 ; c1ccc(-c2nc(-c3cc4ccccc4c4ccccc34)nc(-c3cccc4oc5c(-c6ccc7oc8ccccc8c7c6)cccc5c34)n2)cc1]

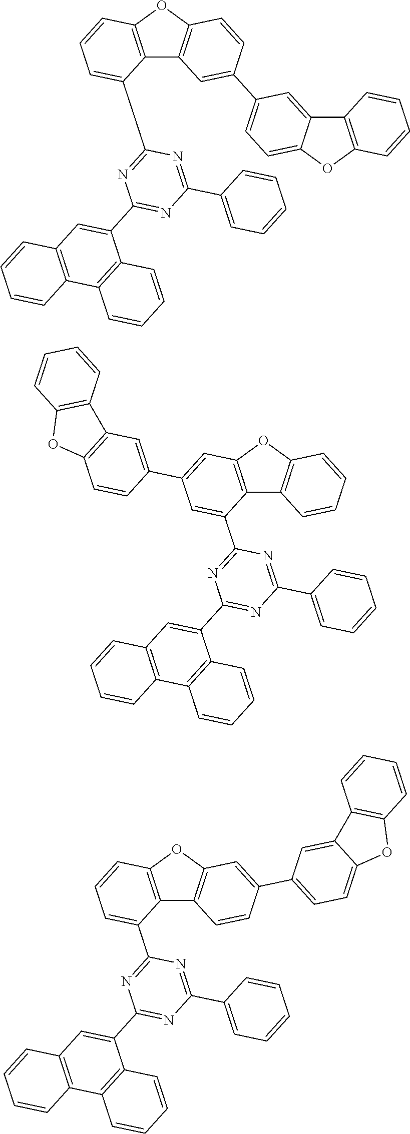

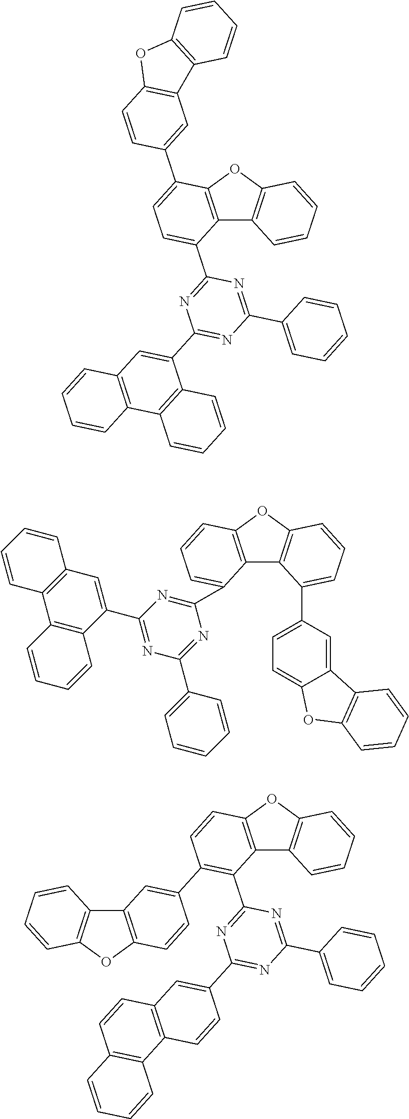 [SMILES: c1ccc(-c2nc(-c3cc4ccccc4c4ccccc34)nc(-c3ccc(-c4ccc5oc6ccccc6c5c4)c4oc5ccccc5c34)n2)cc1 ; c1ccc(-c2nc(-c3cc4ccccc4c4ccccc34)nc(-c3cccc4oc5cccc(-c6ccc7oc8ccccc8c7c6)c5c34)n2)cc1 ; c1ccc(-c2nc(-c3ccc4c(ccc5ccccc54)c3)nc(-c3c(-c4ccc5oc6ccccc6c5c4)ccc4oc5ccccc5c34)n2)cc1]

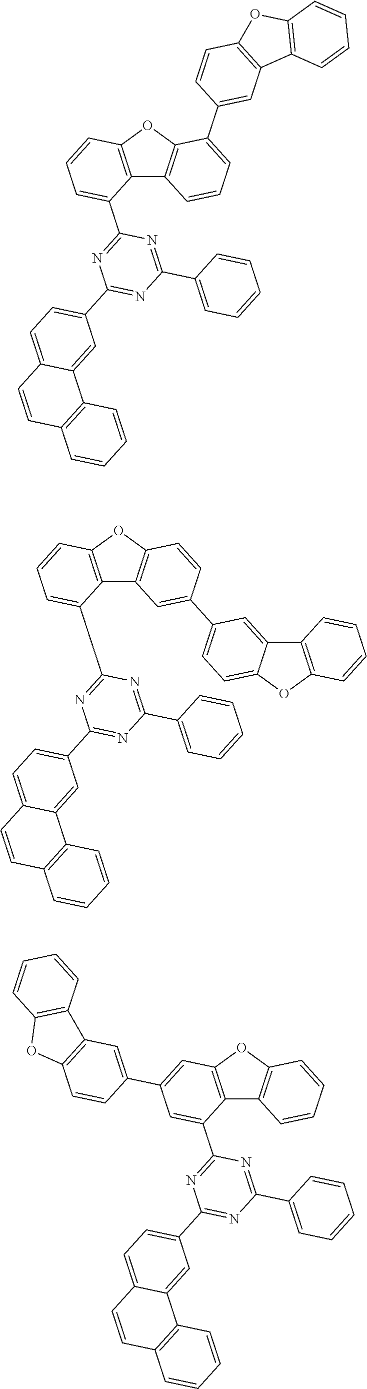 [SMILES: c1ccc(-c2nc(-c3ccc4ccc5ccccc5c4c3)nc(-c3cccc4oc5c(-c6ccc7oc8ccccc8c7c6)cccc5c34)n2)cc1 ; c1ccc(-c2nc(-c3ccc4ccc5ccccc5c4c3)nc(-c3cccc4oc5ccc(-c6ccc7oc8ccccc8c7c6)cc5c34)n2)cc1 ; c1ccc(-c2nc(-c3ccc4ccc5ccccc5c4c3)nc(-c3cc(-c4ccc5oc6ccccc6c5c4)cc4oc5ccccc5c34)n2)cc1]

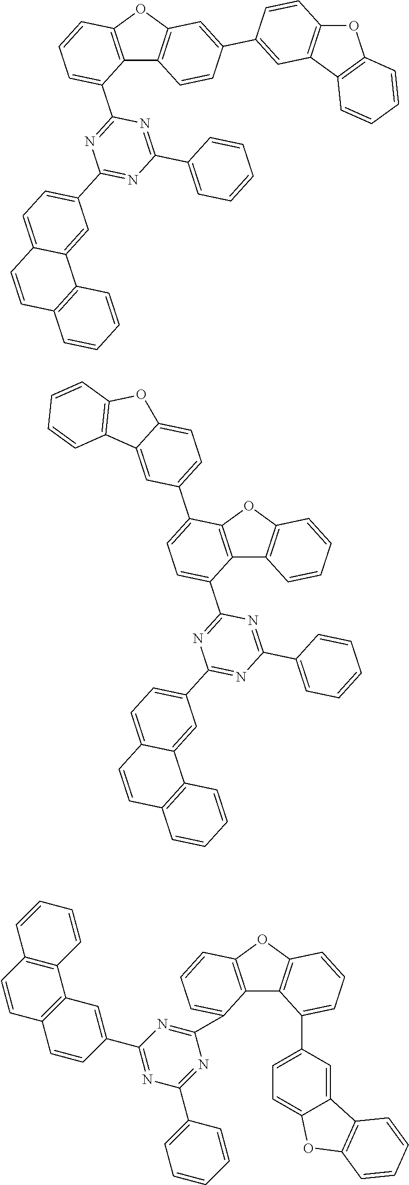

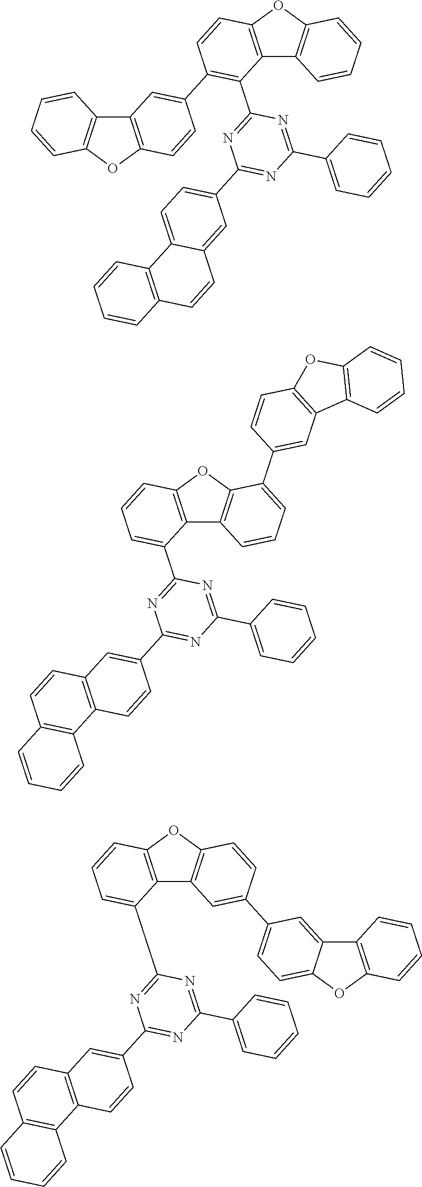 [SMILES: c1ccc(-c2nc(-c3ccc4c(ccc5ccccc54)c3)nc(-c3c(-c4ccc5oc6ccccc6c5c4)ccc4oc5ccccc5c34)n2)cc1 ; c1ccc(-c2nc(-c3ccc4c(ccc5ccccc54)c3)nc(-c3cccc4oc5c(-c6ccc7oc8ccccc8c7c6)cccc5c34)n2)cc1 ; c1ccc(-c2nc(-c3ccc4c(ccc5ccccc54)c3)nc(-c3cccc4oc5ccc(-c6ccc7oc8ccccc8c7c6)cc5c34)n2)cc1]

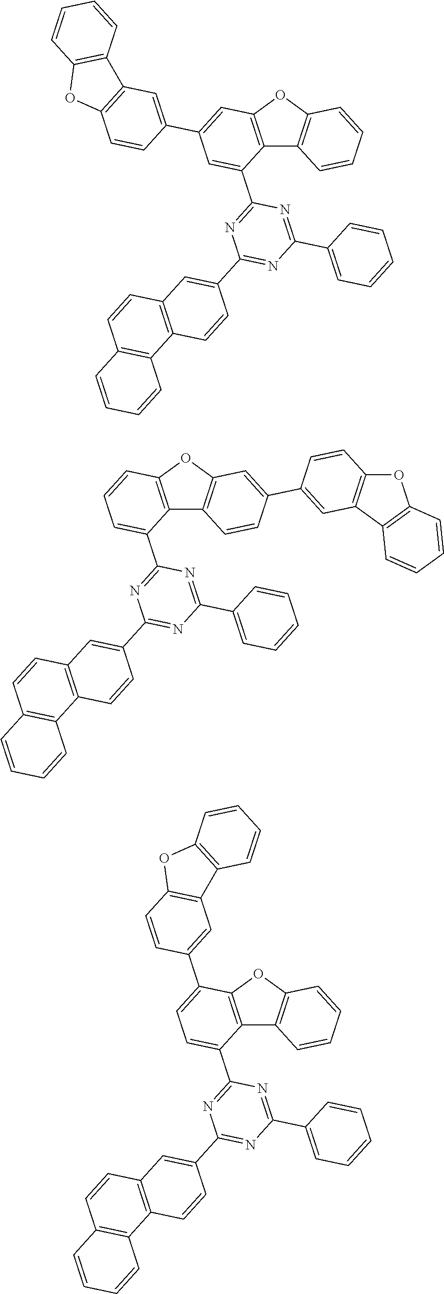 [SMILES: c1ccc(-c2nc(-c3ccc4c(ccc5ccccc54)c3)nc(-c3cc(-c4ccc5oc6ccccc6c5c4)cc4oc5ccccc5c34)n2)cc1 ; c1ccc(-c2nc(-c3ccc4c(ccc5ccccc54)c3)nc(-c3cccc4oc5cc(-c6ccc7oc8ccccc8c7c6)ccc5c34)n2)cc1 ; c1ccc(-c2nc(-c3ccc4c(ccc5ccccc54)c3)nc(-c3ccc(-c4ccc5oc6ccccc6c5c4)c4oc5ccccc5c34)n2)cc1]

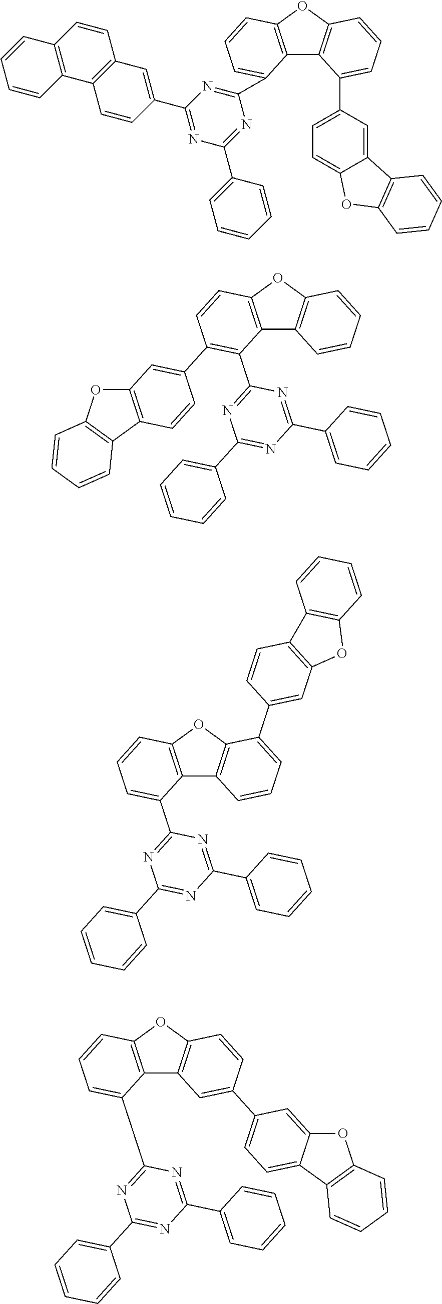 [SMILES: c1ccc(-c2nc(-c3ccc4c(ccc5ccccc54)c3)nc(-c3cccc4oc5cccc(-c6ccc7oc8ccccc8c7c6)c5c34)n2)cc1 ; c1ccc(-c2nc(-c3ccccc3)nc(-c3c(-c4ccc5c(c4)oc4ccccc45)ccc4oc5ccccc5c34)n2)cc1 ; c1ccc(-c2nc(-c3ccccc3)nc(-c3cccc4oc5c(-c6ccc7c(c6)oc6ccccc67)cccc5c34)n2)cc1 ; c1ccc(-c2nc(-c3ccccc3)nc(-c3cccc4oc5ccc(-c6ccc7c(c6)oc6ccccc67)cc5c34)n2)cc1]

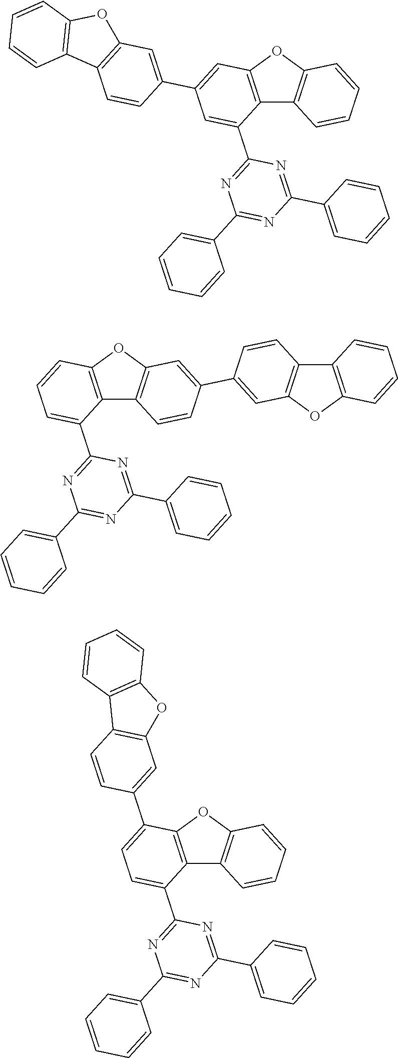 [SMILES: c1ccc(-c2nc(-c3ccccc3)nc(-c3cc(-c4ccc5c(c4)oc4ccccc45)cc4oc5ccccc5c34)n2)cc1 ; c1ccc(-c2nc(-c3ccccc3)nc(-c3cccc4oc5cc(-c6ccc7c(c6)oc6ccccc67)ccc5c34)n2)cc1 ; c1ccc(-c2nc(-c3ccccc3)nc(-c3ccc(-c4ccc5c(c4)oc4ccccc45)c4oc5ccccc5c34)n2)cc1]

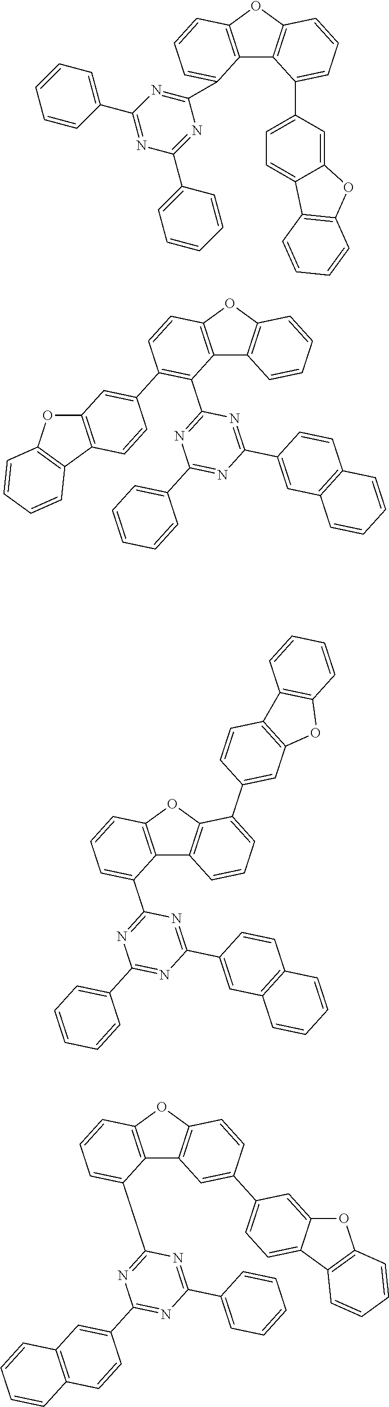

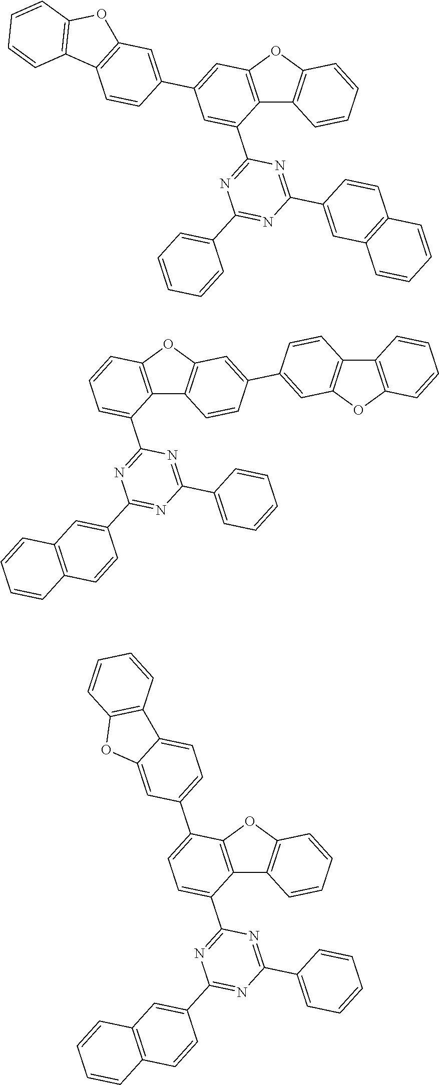 [SMILES: c1ccc(-c2nc(-c3ccc4ccccc4c3)nc(-c3cc(-c4ccc5c(c4)oc4ccccc45)cc4oc5ccccc5c34)n2)cc1 ; c1ccc(-c2nc(-c3ccc4ccccc4c3)nc(-c3cccc4oc5cc(-c6ccc7c(c6)oc6ccccc67)ccc5c34)n2)cc1 ; c1ccc(-c2nc(-c3ccc4ccccc4c3)nc(-c3ccc(-c4ccc5c(c4)oc4ccccc45)c4oc5ccccc5c34)n2)cc1]

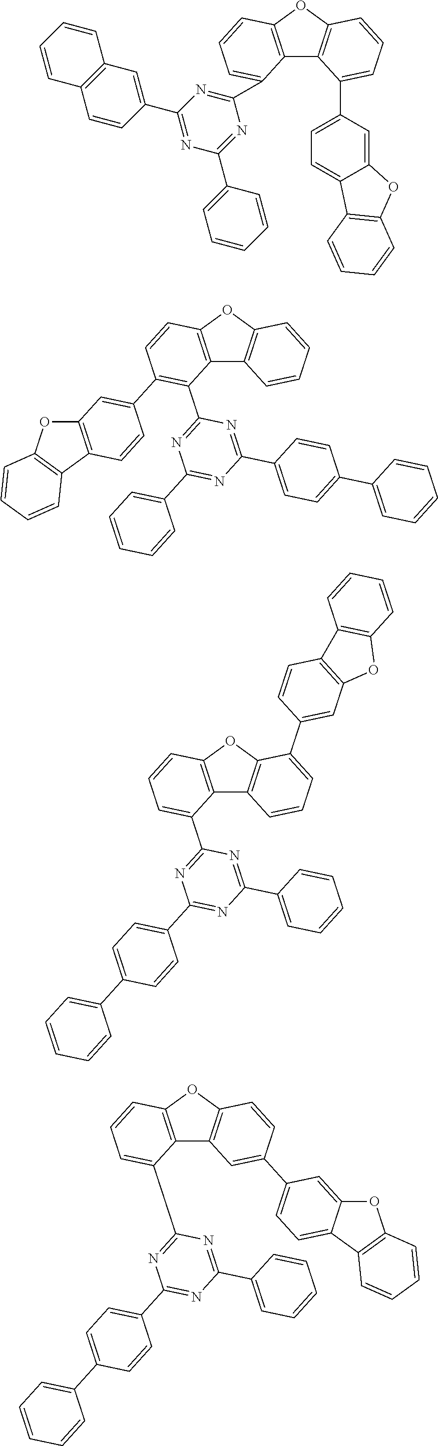 [SMILES: c1ccc(-c2nc(-c3ccc4ccccc4c3)nc(-c3cccc4oc5cccc(-c6ccc7c(c6)oc6ccccc67)c5c34)n2)cc1 ; c1ccc(-c2ccc(-c3nc(-c4ccccc4)nc(-c4c(-c5ccc6c(c5)oc5ccccc56)ccc5oc6ccccc6c45)n3)cc2)cc1 ; c1ccc(-c2ccc(-c3nc(-c4ccccc4)nc(-c4cccc5oc6c(-c7ccc8c(c7)oc7ccccc78)cccc6c45)n3)cc2)cc1 ; c1ccc(-c2ccc(-c3nc(-c4ccccc4)nc(-c4cccc5oc6ccc(-c7ccc8c(c7)oc7ccccc78)cc6c45)n3)cc2)cc1]

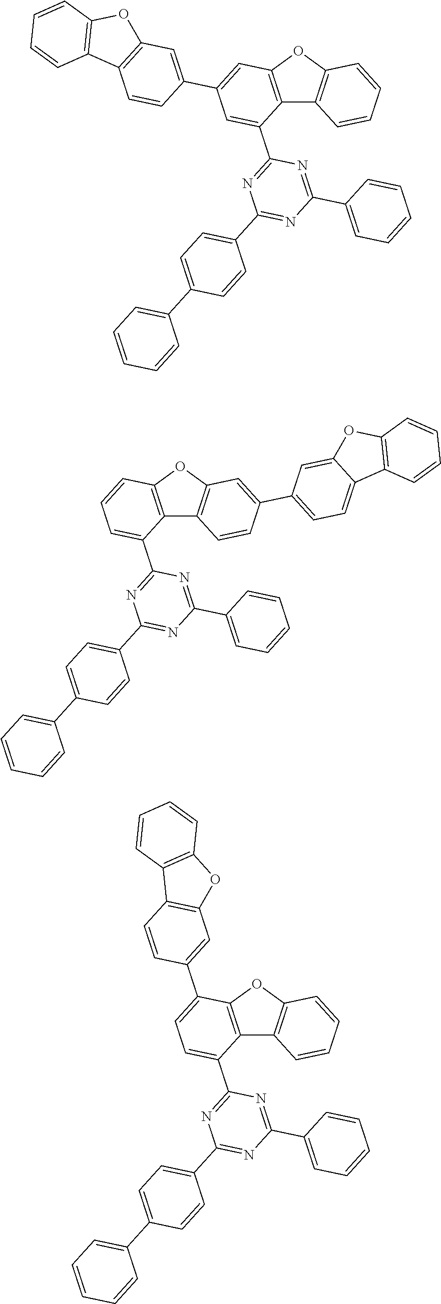 [SMILES: c1ccc(-c2ccc(-c3nc(-c4ccccc4)nc(-c4cc(-c5ccc6c(c5)oc5ccccc56)cc5oc6ccccc6c45)n3)cc2)cc1 ; c1ccc(-c2ccc(-c3nc(-c4ccccc4)nc(-c4cccc5oc6cc(-c7ccc8c(c7)oc7ccccc78)ccc6c45)n3)cc2)cc1 ; c1ccc(-c2ccc(-c3nc(-c4ccccc4)nc(-c4ccc(-c5ccc6c(c5)oc5ccccc56)c5oc6ccccc6c45)n3)cc2)cc1]

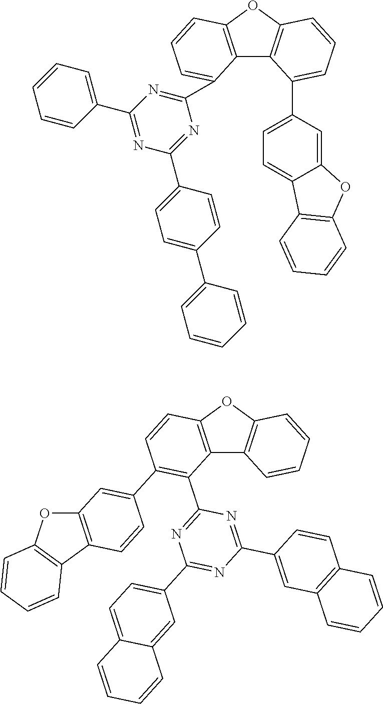 [SMILES: c1ccc(-c2ccc(-c3nc(-c4ccccc4)nc(-c4cccc5oc6cccc(-c7ccc8c(c7)oc7ccccc78)c6c45)n3)cc2)cc1 ; c1ccc2cc(-c3nc(-c4ccc5ccccc5c4)nc(-c4c(-c5ccc6c(c5)oc5ccccc56)ccc5oc6ccccc6c45)n3)ccc2c1]

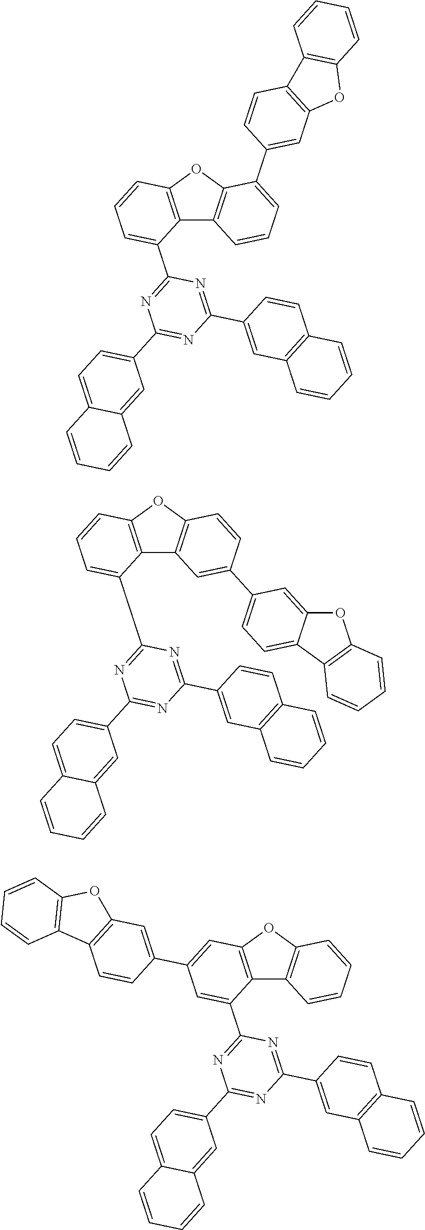

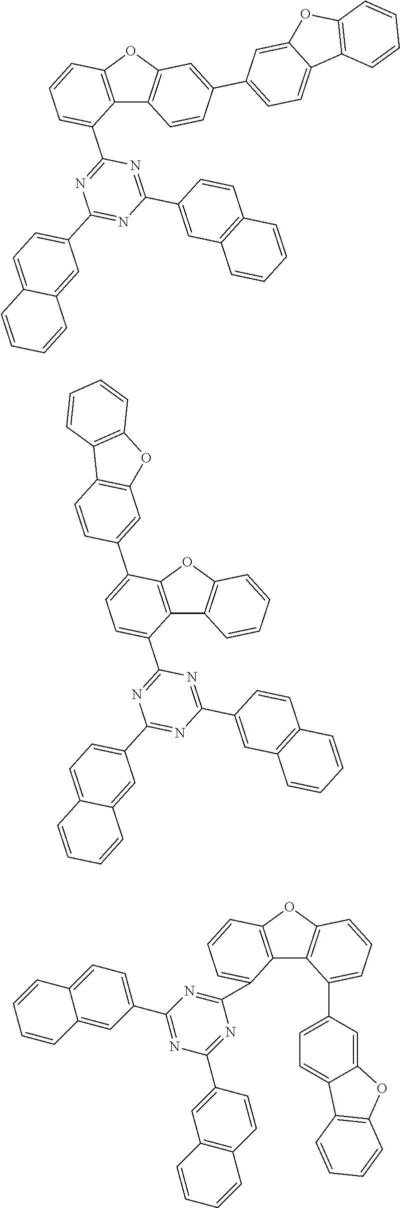 [SMILES: c1ccc2cc(-c3nc(-c4ccc5ccccc5c4)nc(-c4cccc5oc6cc(-c7ccc8c(c7)oc7ccccc78)ccc6c45)n3)ccc2c1 ; c1ccc2cc(-c3nc(-c4ccc5ccccc5c4)nc(-c4ccc(-c5ccc6c(c5)oc5ccccc56)c5oc6ccccc6c45)n3)ccc2c1 ; c1ccc2cc(-c3nc(-c4ccc5ccccc5c4)nc(-c4cccc5oc6cccc(-c7ccc8c(c7)oc7ccccc78)c6c45)n3)ccc2c1]

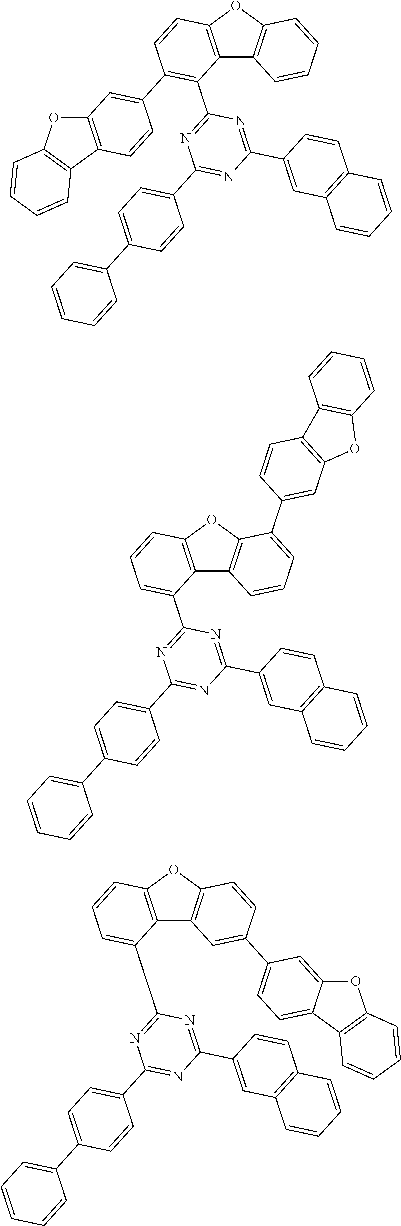 [SMILES: c1ccc(-c2ccc(-c3nc(-c4ccc5ccccc5c4)nc(-c4c(-c5ccc6c(c5)oc5ccccc56)ccc5oc6ccccc6c45)n3)cc2)cc1 ; c1ccc(-c2ccc(-c3nc(-c4ccc5ccccc5c4)nc(-c4cccc5oc6c(-c7ccc8c(c7)oc7ccccc78)cccc6c45)n3)cc2)cc1 ; c1ccc(-c2ccc(-c3nc(-c4ccc5ccccc5c4)nc(-c4cccc5oc6ccc(-c7ccc8c(c7)oc7ccccc78)cc6c45)n3)cc2)cc1]

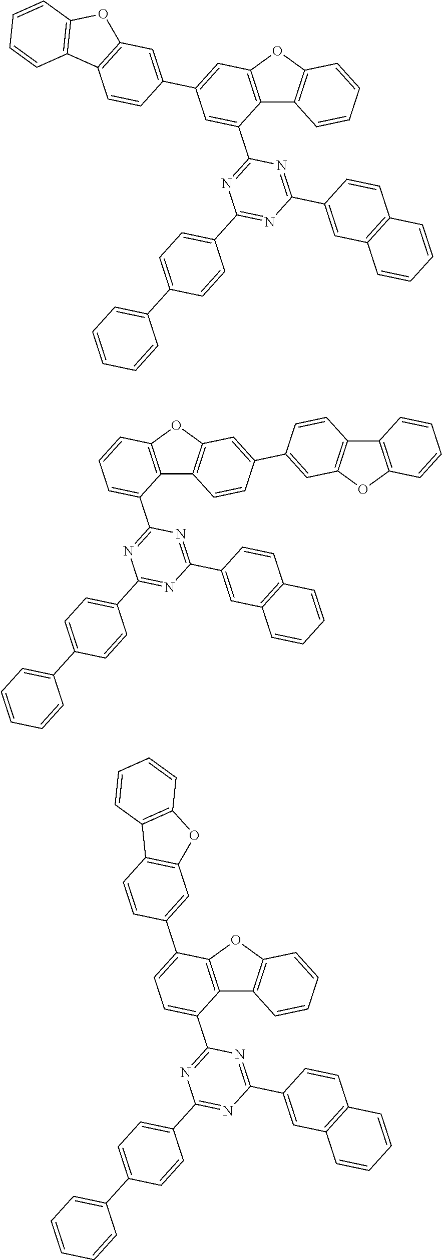 [SMILES: c1ccc(-c2ccc(-c3nc(-c4ccc5ccccc5c4)nc(-c4cc(-c5ccc6c(c5)oc5ccccc56)cc5oc6ccccc6c45)n3)cc2)cc1 ; c1ccc(-c2ccc(-c3nc(-c4ccc5ccccc5c4)nc(-c4cccc5oc6cc(-c7ccc8c(c7)oc7ccccc78)ccc6c45)n3)cc2)cc1 ; c1ccc(-c2ccc(-c3nc(-c4ccc5ccccc5c4)nc(-c4ccc(-c5ccc6c(c5)oc5ccccc56)c5oc6ccccc6c45)n3)cc2)cc1]

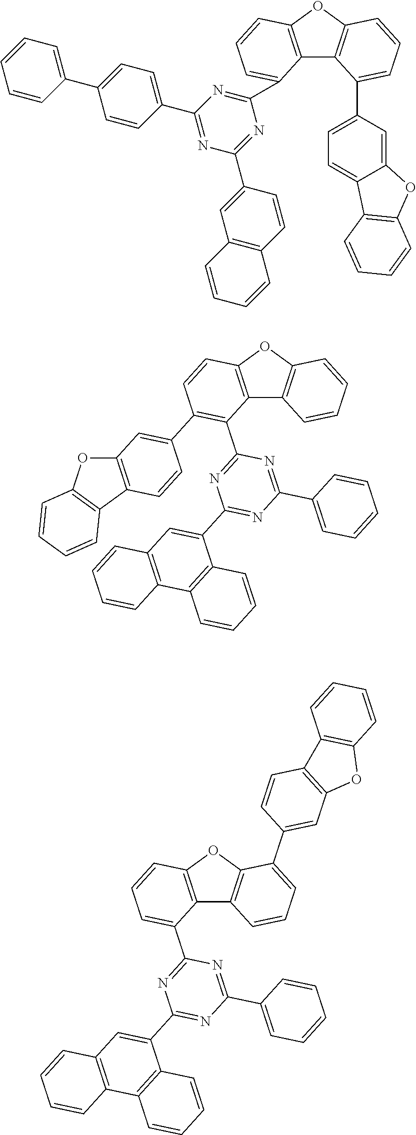

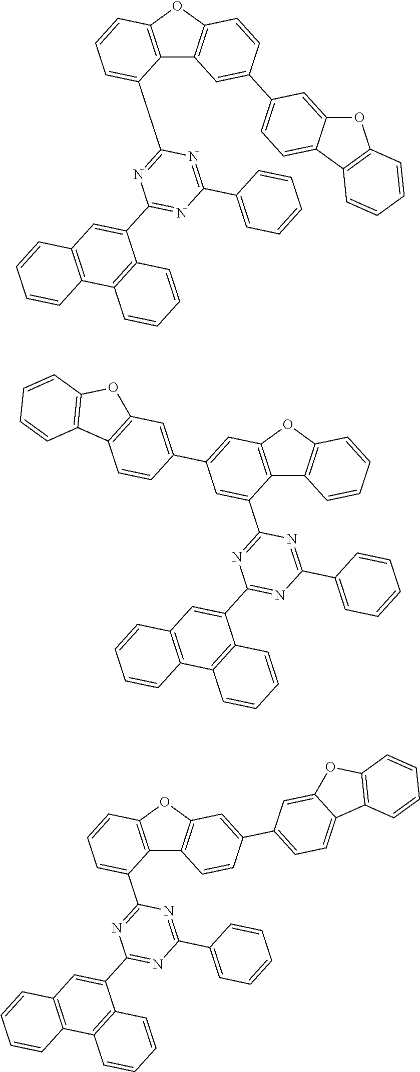 [SMILES: c1ccc(-c2nc(-c3cc4ccccc4c4ccccc34)nc(-c3cccc4oc5ccc(-c6ccc7c(c6)oc6ccccc67)cc5c34)n2)cc1 ; c1ccc(-c2nc(-c3cc4ccccc4c4ccccc34)nc(-c3cc(-c4ccc5c(c4)oc4ccccc45)cc4oc5ccccc5c34)n2)cc1 ; c1ccc(-c2nc(-c3cc4ccccc4c4ccccc34)nc(-c3cccc4oc5cc(-c6ccc7c(c6)oc6ccccc67)ccc5c34)n2)cc1]

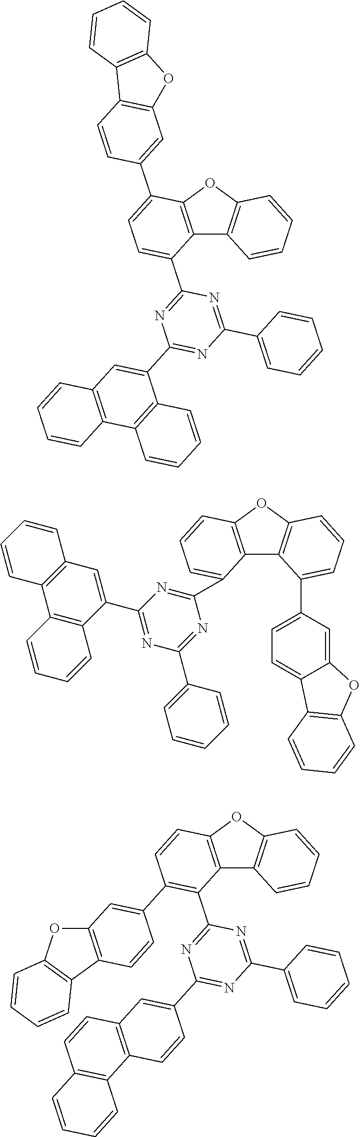 [SMILES: c1ccc(-c2nc(-c3cc4ccccc4c4ccccc34)nc(-c3ccc(-c4ccc5c(c4)oc4ccccc45)c4oc5ccccc5c34)n2)cc1 ; c1ccc(-c2nc(-c3cc4ccccc4c4ccccc34)nc(-c3cccc4oc5cccc(-c6ccc7c(c6)oc6ccccc67)c5c34)n2)cc1 ; c1ccc(-c2nc(-c3ccc4c(ccc5ccccc54)c3)nc(-c3c(-c4ccc5c(c4)oc4ccccc45)ccc4oc5ccccc5c34)n2)cc1]

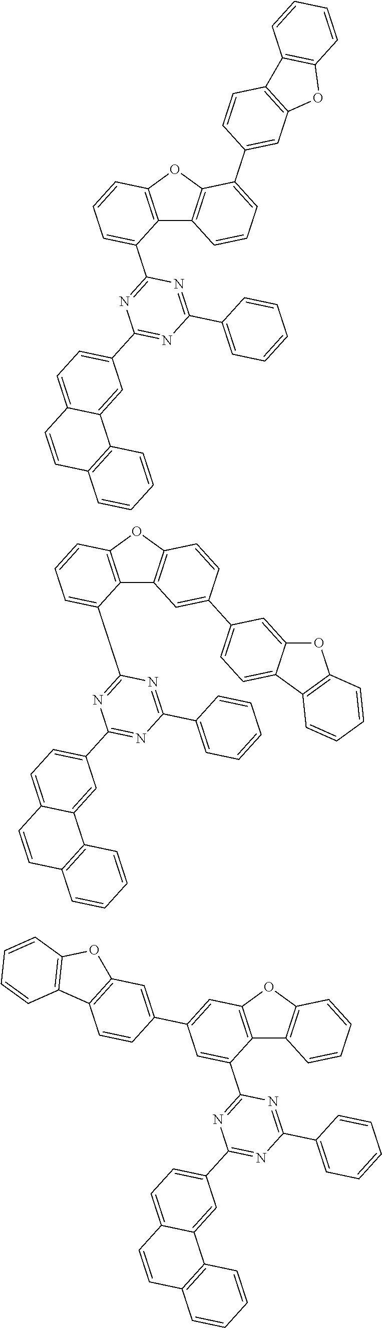

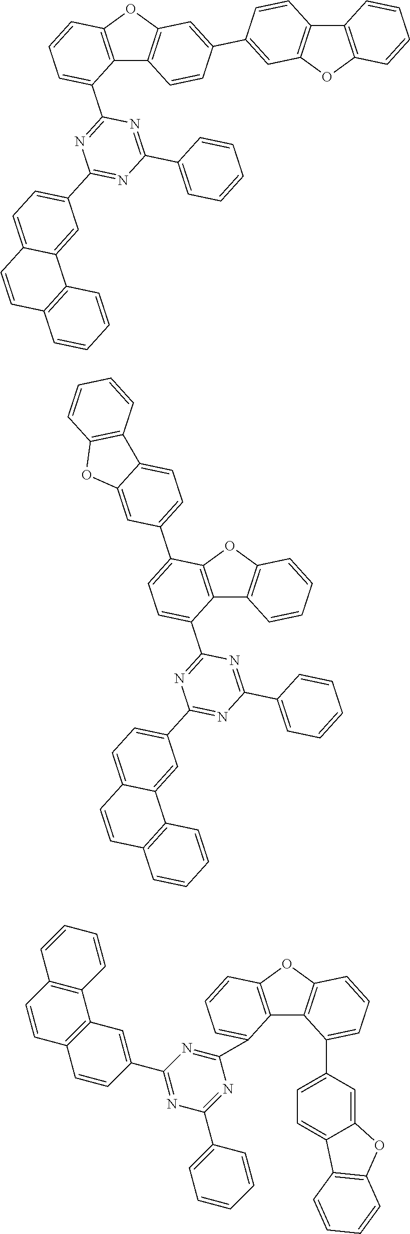

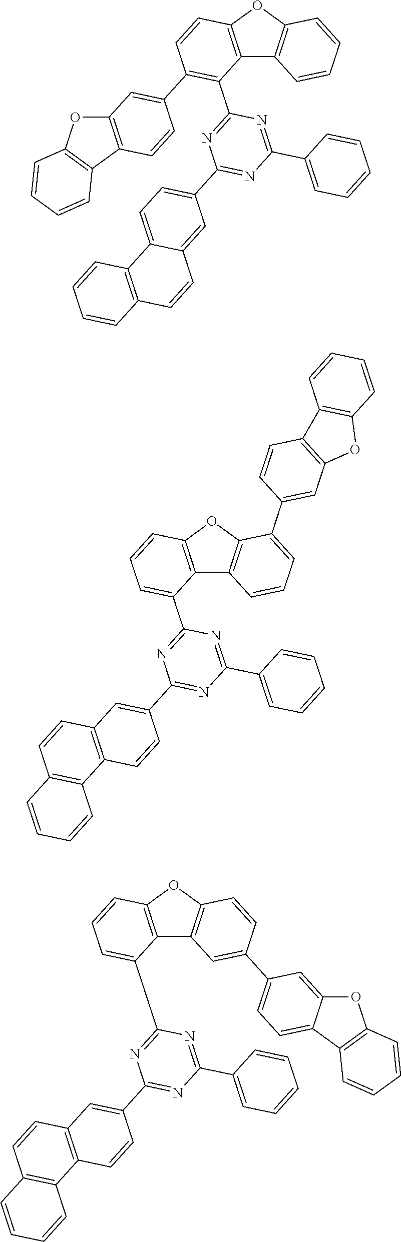 [SMILES: c1ccc(-c2nc(-c3ccc4c(ccc5ccccc54)c3)nc(-c3c(-c4ccc5c(c4)oc4ccccc45)ccc4oc5ccccc5c34)n2)cc1 ; c1ccc(-c2nc(-c3ccc4c(ccc5ccccc54)c3)nc(-c3cccc4oc5c(-c6ccc7c(c6)oc6ccccc67)cccc5c34)n2)cc1 ; c1ccc(-c2nc(-c3ccc4c(ccc5ccccc54)c3)nc(-c3cccc4oc5ccc(-c6ccc7c(c6)oc6ccccc67)cc5c34)n2)cc1]

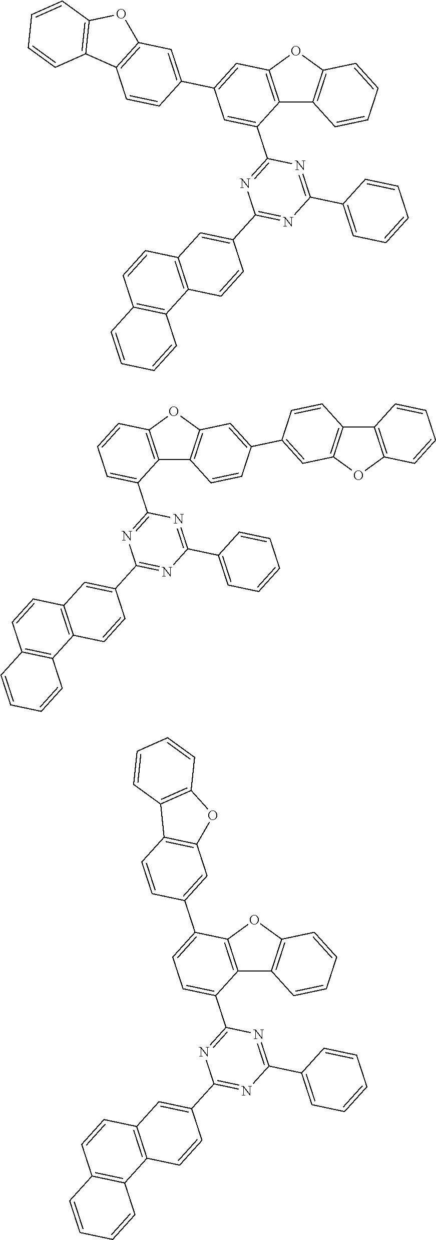

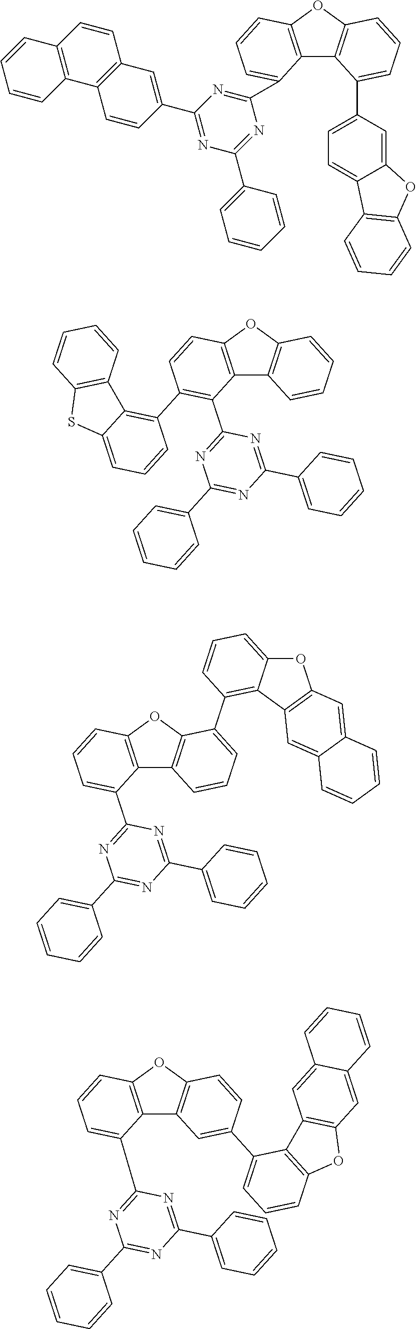 [SMILES: c1ccc(-c2nc(-c3ccc4c(ccc5ccccc54)c3)nc(-c3cccc4oc5cccc(-c6ccc7c(c6)oc6ccccc67)c5c34)n2)cc1 ; c1ccc(-c2nc(-c3ccccc3)nc(-c3c(-c4cccc5sc6ccccc6c45)ccc4oc5ccccc5c34)n2)cc1 ; c1ccc(-c2nc(-c3ccccc3)nc(-c3cccc4oc5c(-c6cccc7oc8cc9ccccc9cc8c67)cccc5c34)n2)cc1 ; c1ccc(-c2nc(-c3ccccc3)nc(-c3cccc4oc5ccc(-c6cccc7oc8cc9ccccc9cc8c67)cc5c34)n2)cc1]

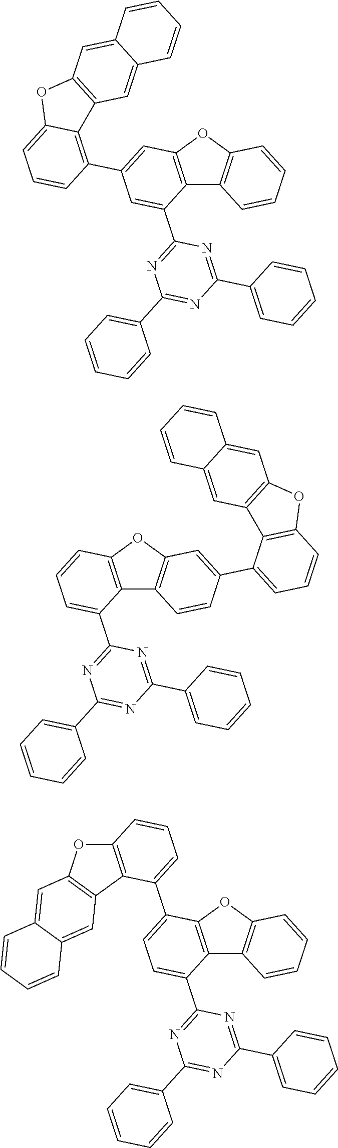 [SMILES: c1ccc(-c2nc(-c3ccccc3)nc(-c3cc(-c4cccc5oc6cc7ccccc7cc6c45)cc4oc5ccccc5c34)n2)cc1 ; c1ccc(-c2nc(-c3ccccc3)nc(-c3cccc4oc5cc(-c6cccc7oc8cc9ccccc9cc8c67)ccc5c34)n2)cc1 ; c1ccc(-c2nc(-c3ccccc3)nc(-c3ccc(-c4cccc5oc6cc7ccccc7cc6c45)c4oc5ccccc5c34)n2)cc1]

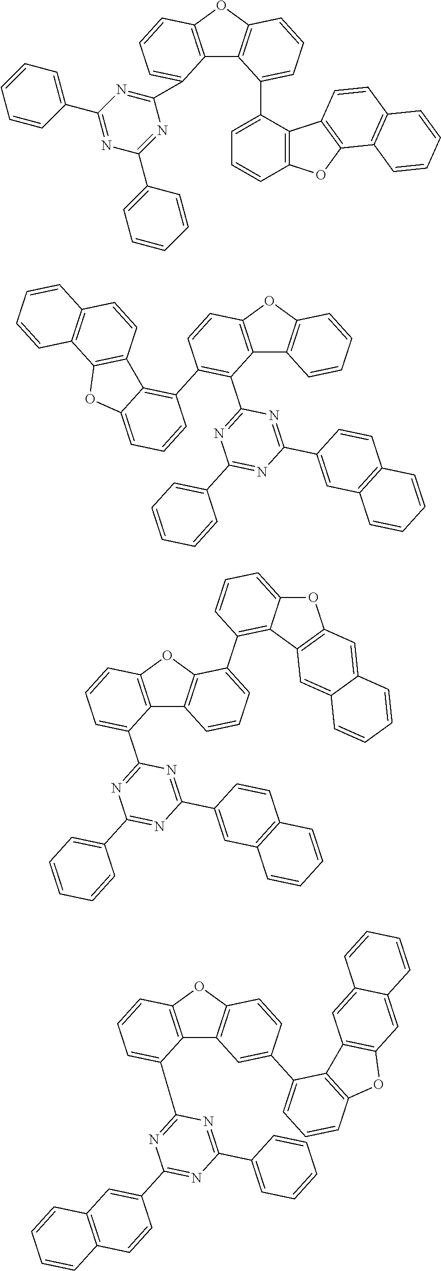

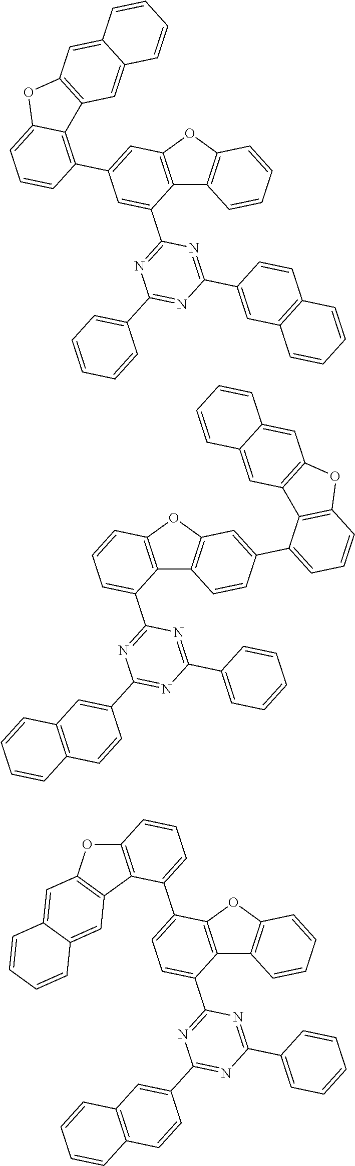 [SMILES: c1ccc(-c2nc(-c3ccc4ccccc4c3)nc(-c3cc(-c4cccc5oc6cc7ccccc7cc6c45)cc4oc5ccccc5c34)n2)cc1 ; c1ccc(-c2nc(-c3ccc4ccccc4c3)nc(-c3cccc4oc5cc(-c6cccc7oc8cc9ccccc9cc8c67)ccc5c34)n2)cc1 ; c1ccc(-c2nc(-c3ccc4ccccc4c3)nc(-c3ccc(-c4cccc5oc6cc7ccccc7cc6c45)c4oc5ccccc5c34)n2)cc1]

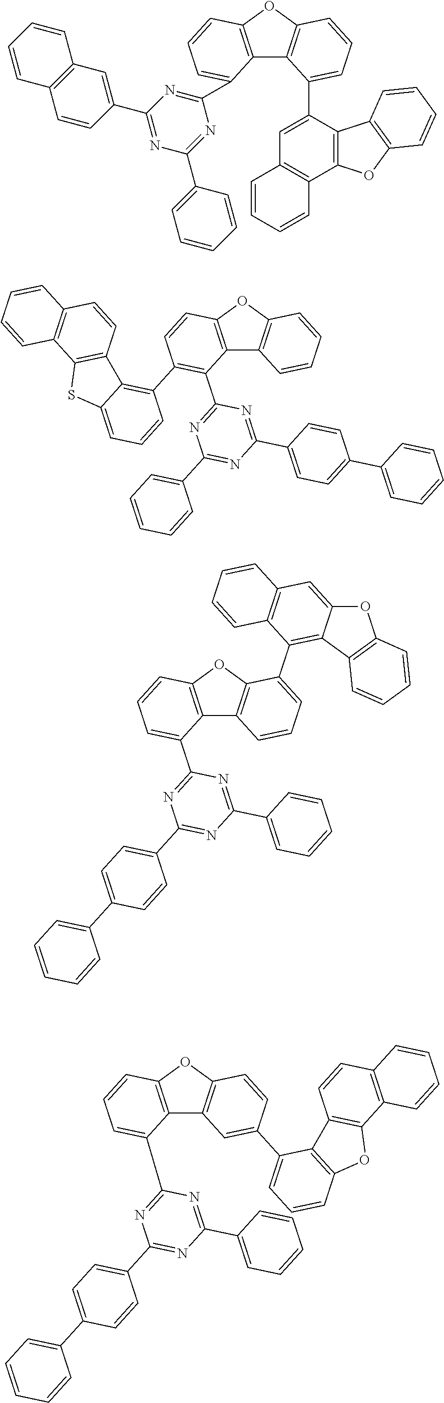 [SMILES: c1ccc(-c2nc(-c3ccc4ccccc4c3)nc(-c3cccc4oc5cccc(-c6cc7ccccc7c7oc8ccccc8c67)c5c34)n2)cc1 ; c1ccc(-c2ccc(-c3nc(-c4ccccc4)nc(-c4c(-c5cccc6sc7c8ccccc8ccc7c56)ccc5oc6ccccc6c45)n3)cc2)cc1 ; c1ccc(-c2ccc(-c3nc(-c4ccccc4)nc(-c4cccc5oc6c(-c7c8ccccc8cc8oc9ccccc9c78)cccc6c45)n3)cc2)cc1 ; c1ccc(-c2ccc(-c3nc(-c4ccccc4)nc(-c4cccc5oc6ccc(-c7cccc8oc9c%10ccccc%10ccc9c78)cc6c45)n3)cc2)cc1]

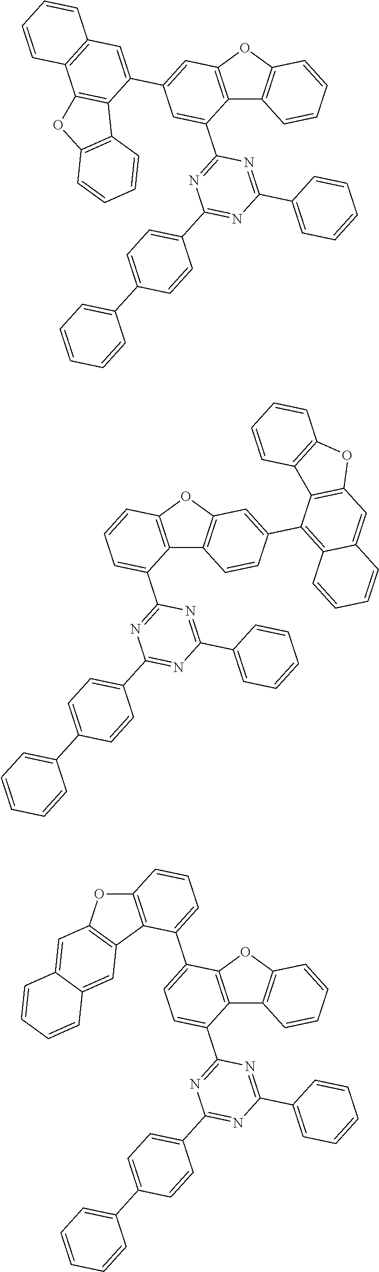 [SMILES: c1ccc(-c2ccc(-c3nc(-c4ccccc4)nc(-c4cc(-c5cc6ccccc6c6oc7ccccc7c56)cc5oc6ccccc6c45)n3)cc2)cc1 ; c1ccc(-c2ccc(-c3nc(-c4ccccc4)nc(-c4cccc5oc6cc(-c7c8ccccc8cc8oc9ccccc9c78)ccc6c45)n3)cc2)cc1 ; c1ccc(-c2ccc(-c3nc(-c4ccccc4)nc(-c4ccc(-c5cccc6oc7cc8ccccc8cc7c56)c5oc6ccccc6c45)n3)cc2)cc1]

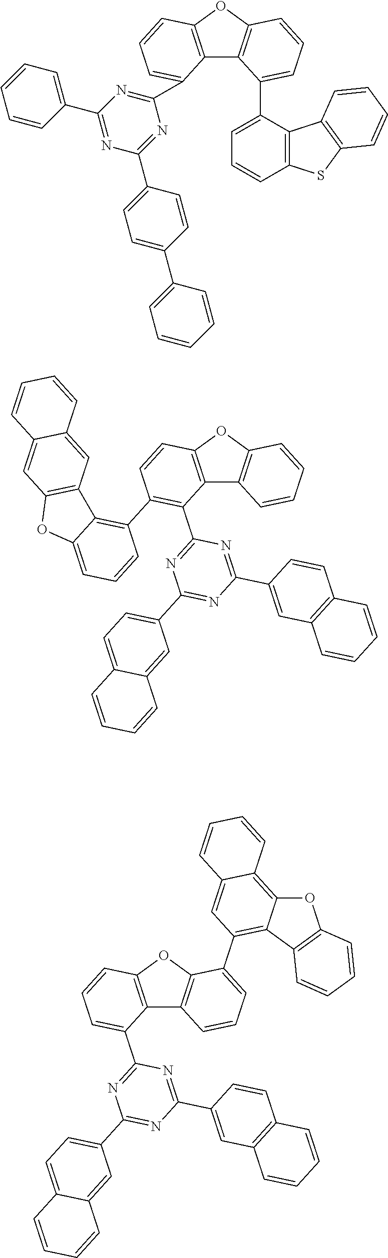 [SMILES: c1ccc(-c2ccc(-c3nc(-c4ccccc4)nc(-c4cccc5oc6cccc(-c7cccc8sc9ccccc9c78)c6c45)n3)cc2)cc1 ; c1ccc2cc(-c3nc(-c4ccc5ccccc5c4)nc(-c4c(-c5cccc6oc7cc8ccccc8cc7c56)ccc5oc6ccccc6c45)n3)ccc2c1 ; c1ccc2cc(-c3nc(-c4ccc5ccccc5c4)nc(-c4cccc5oc6c(-c7cc8ccccc8c8oc9ccccc9c78)cccc6c45)n3)ccc2c1]

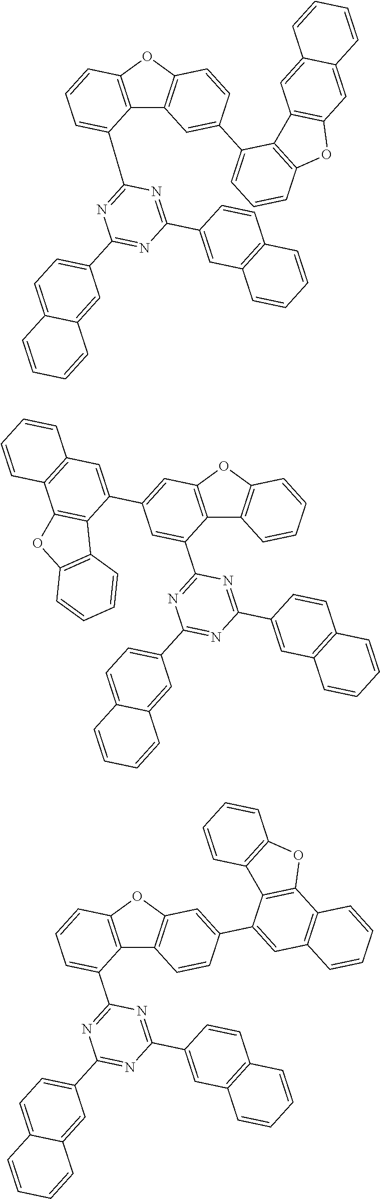 [SMILES: c1ccc2cc(-c3nc(-c4ccc5ccccc5c4)nc(-c4cccc5oc6ccc(-c7cccc8oc9cc%10ccccc%10cc9c78)cc6c45)n3)ccc2c1 ; c1ccc2cc(-c3nc(-c4ccc5ccccc5c4)nc(-c4cc(-c5cc6ccccc6c6oc7ccccc7c56)cc5oc6ccccc6c45)n3)ccc2c1 ; c1ccc2cc(-c3nc(-c4ccc5ccccc5c4)nc(-c4cccc5oc6cc(-c7cc8ccccc8c8oc9ccccc9c78)ccc6c45)n3)ccc2c1]

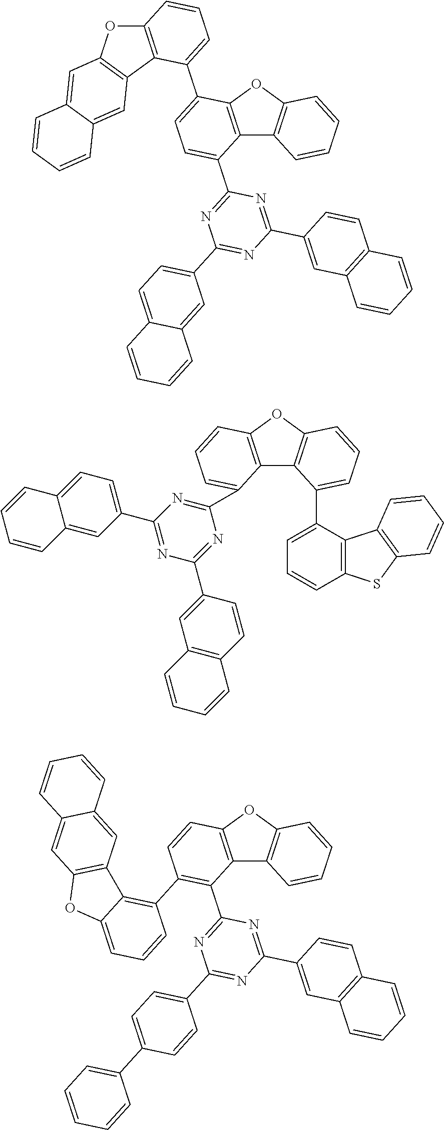 [SMILES: c1ccc2cc(-c3nc(-c4ccc5ccccc5c4)nc(-c4ccc(-c5cccc6oc7cc8ccccc8cc7c56)c5oc6ccccc6c45)n3)ccc2c1 ; c1ccc2cc(-c3nc(-c4ccc5ccccc5c4)nc(-c4cccc5oc6cccc(-c7cccc8sc9ccccc9c78)c6c45)n3)ccc2c1 ; c1ccc(-c2ccc(-c3nc(-c4ccc5ccccc5c4)nc(-c4c(-c5cccc6oc7cc8ccccc8cc7c56)ccc5oc6ccccc6c45)n3)cc2)cc1]

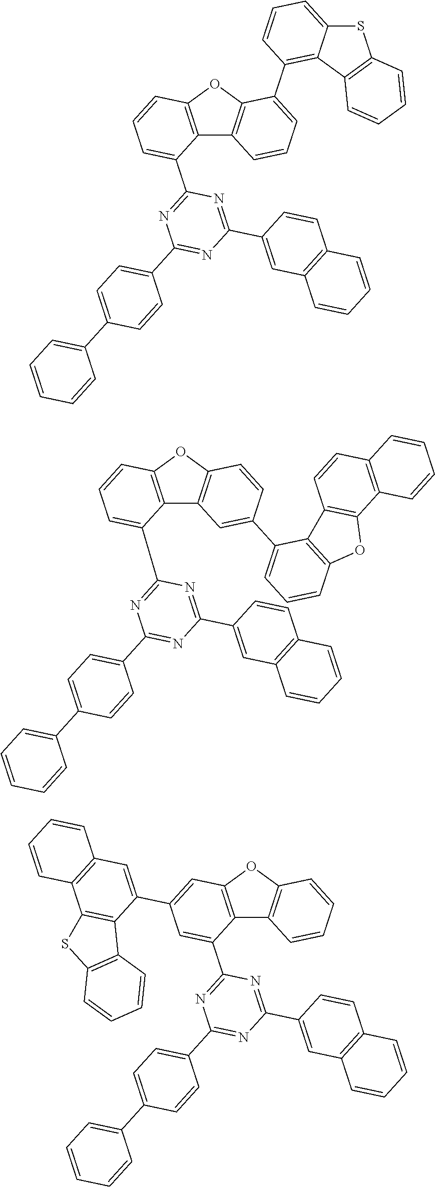

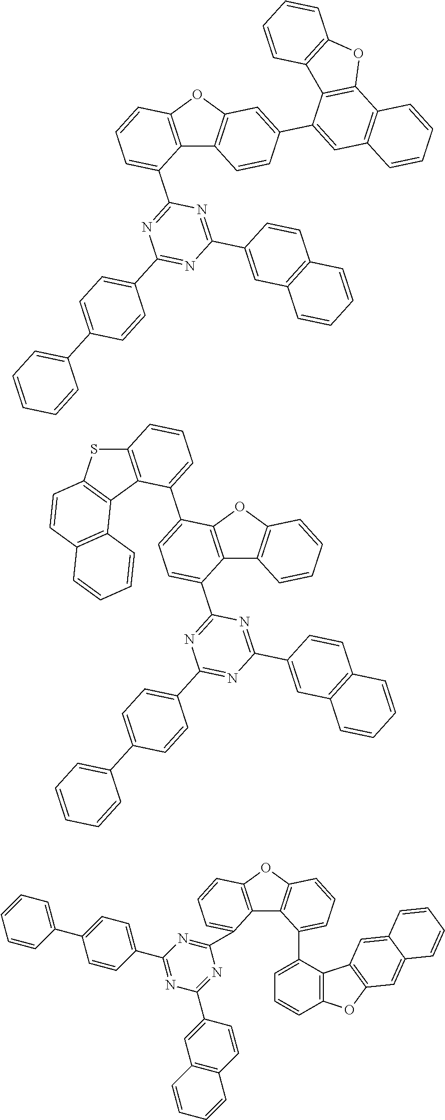 [SMILES: c1ccc(-c2ccc(-c3nc(-c4ccc5ccccc5c4)nc(-c4cccc5oc6cc(-c7cc8ccccc8c8oc9ccccc9c78)ccc6c45)n3)cc2)cc1 ; c1ccc(-c2ccc(-c3nc(-c4ccc5ccccc5c4)nc(-c4ccc(-c5cccc6sc7ccc8ccccc8c7c56)c5oc6ccccc6c45)n3)cc2)cc1 ; c1ccc(-c2ccc(-c3nc(-c4ccc5ccccc5c4)nc(-c4cccc5oc6cccc(-c7cccc8oc9cc%10ccccc%10cc9c78)c6c45)n3)cc2)cc1]

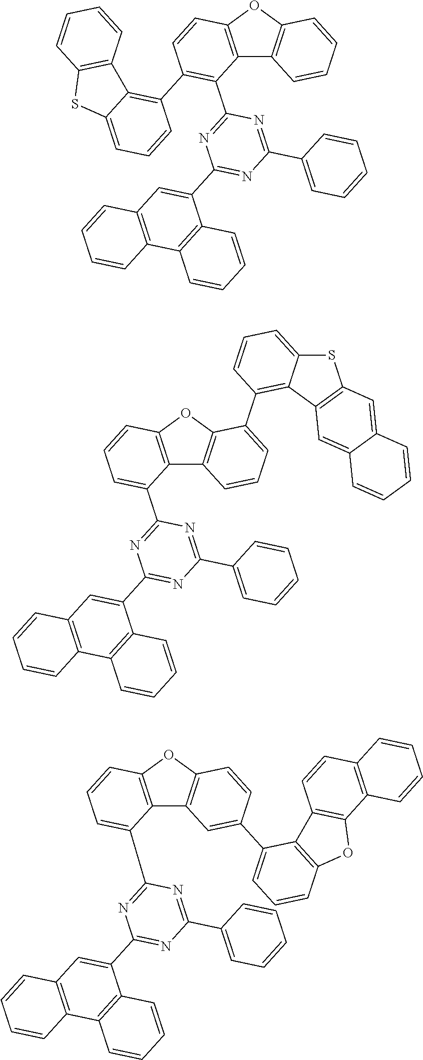 [SMILES: c1ccc(-c2nc(-c3cc4ccccc4c4ccccc34)nc(-c3c(-c4cccc5sc6ccccc6c45)ccc4oc5ccccc5c34)n2)cc1 ; c1ccc(-c2nc(-c3cc4ccccc4c4ccccc34)nc(-c3cccc4oc5c(-c6cccc7sc8cc9ccccc9cc8c67)cccc5c34)n2)cc1 ; c1ccc(-c2nc(-c3cc4ccccc4c4ccccc34)nc(-c3cccc4oc5ccc(-c6cccc7oc8c9ccccc9ccc8c67)cc5c34)n2)cc1]

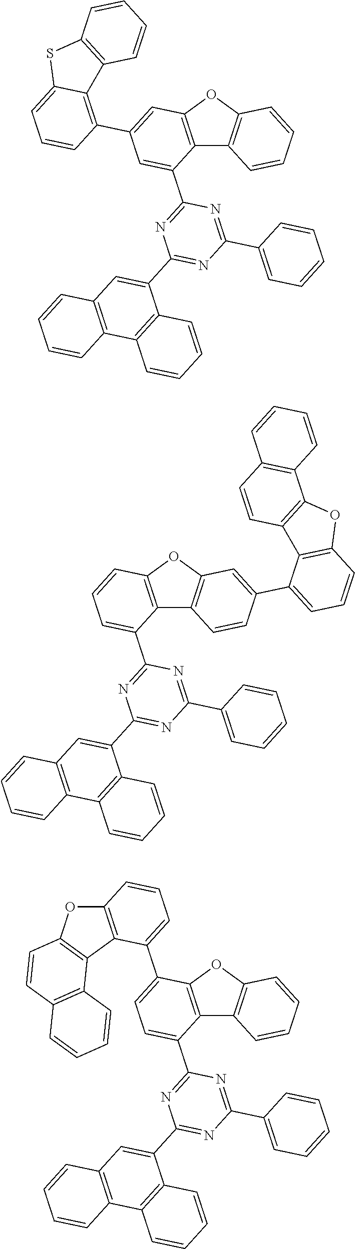

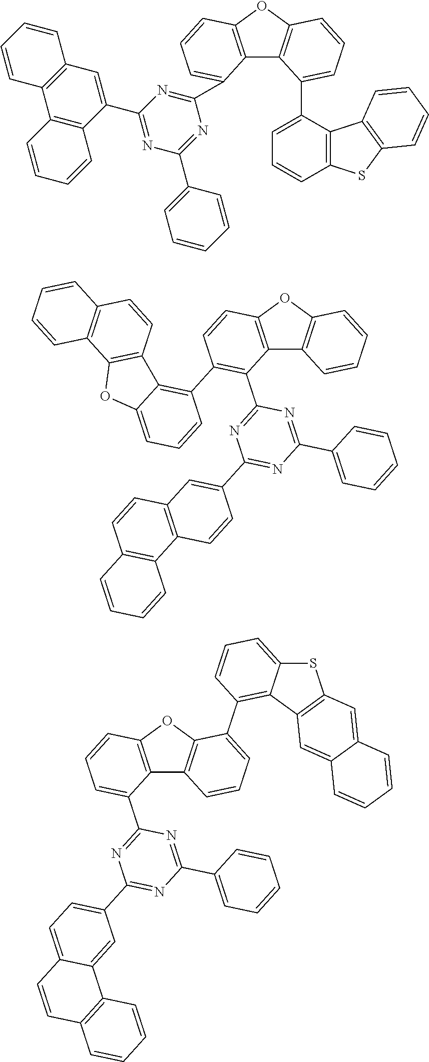 [SMILES: c1ccc(-c2nc(-c3cc4ccccc4c4ccccc34)nc(-c3cccc4oc5cccc(-c6cccc7sc8ccccc8c67)c5c34)n2)cc1 ; c1ccc(-c2nc(-c3ccc4c(ccc5ccccc54)c3)nc(-c3c(-c4cccc5oc6c7ccccc7ccc6c45)ccc4oc5ccccc5c34)n2)cc1 ; c1ccc(-c2nc(-c3ccc4ccc5ccccc5c4c3)nc(-c3cccc4oc5c(-c6cccc7sc8cc9ccccc9cc8c67)cccc5c34)n2)cc1]

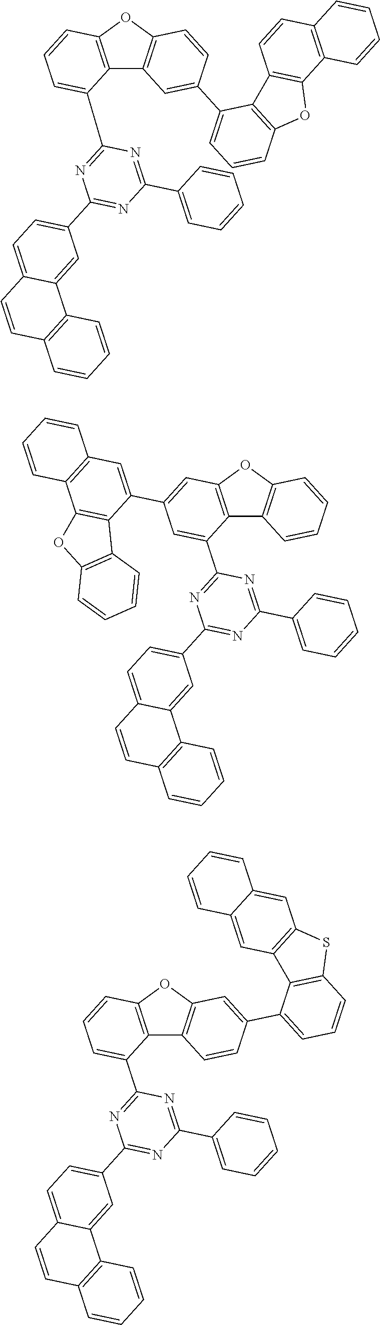 [SMILES: c1ccc(-c2nc(-c3ccc4ccc5ccccc5c4c3)nc(-c3cccc4oc5ccc(-c6cccc7oc8c9ccccc9ccc8c67)cc5c34)n2)cc1 ; c1ccc(-c2nc(-c3ccc4ccc5ccccc5c4c3)nc(-c3cc(-c4cc5ccccc5c5oc6ccccc6c45)cc4oc5ccccc5c34)n2)cc1 ; c1ccc(-c2nc(-c3ccc4ccc5ccccc5c4c3)nc(-c3cccc4oc5cc(-c6cccc7sc8cc9ccccc9cc8c67)ccc5c34)n2)cc1]

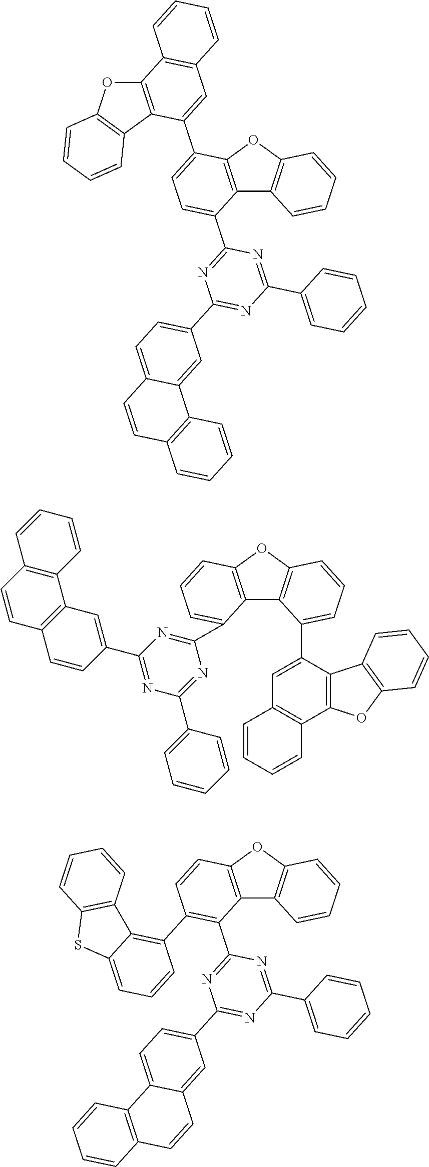 [SMILES: c1ccc(-c2nc(-c3ccc4ccc5ccccc5c4c3)nc(-c3ccc(-c4cc5ccccc5c5oc6ccccc6c45)c4oc5ccccc5c34)n2)cc1 ; c1ccc(-c2nc(-c3ccc4ccc5ccccc5c4c3)nc(-c3cccc4oc5cccc(-c6cc7ccccc7c7oc8ccccc8c67)c5c34)n2)cc1 ; c1ccc(-c2nc(-c3ccc4c(ccc5ccccc54)c3)nc(-c3c(-c4cccc5sc6ccccc6c45)ccc4oc5ccccc5c34)n2)cc1]

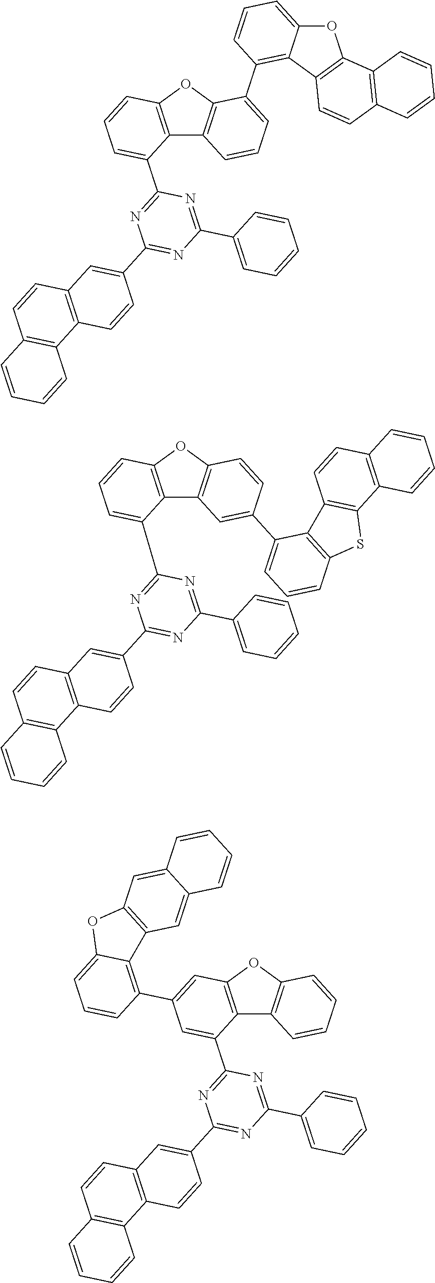 [SMILES: c1ccc(-c2nc(-c3ccc4c(ccc5ccccc54)c3)nc(-c3cccc4oc5c(-c6cccc7oc8c9ccccc9ccc8c67)cccc5c34)n2)cc1 ; c1ccc(-c2nc(-c3ccc4c(ccc5ccccc54)c3)nc(-c3cccc4oc5ccc(-c6cccc7sc8c9ccccc9ccc8c67)cc5c34)n2)cc1 ; c1ccc(-c2nc(-c3ccc4c(ccc5ccccc54)c3)nc(-c3cc(-c4cccc5oc6cc7ccccc7cc6c45)cc4oc5ccccc5c34)n2)cc1]

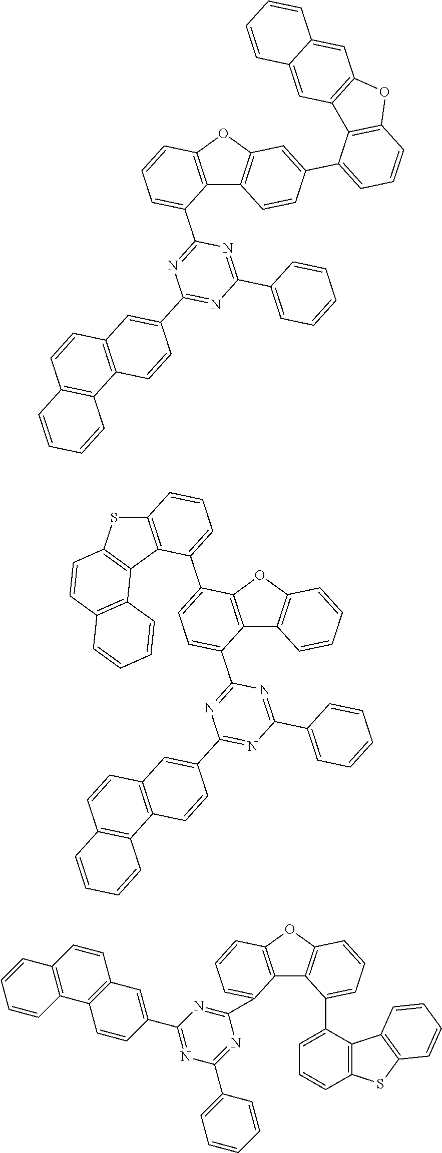 [SMILES: c1ccc(-c2nc(-c3ccc4c(ccc5ccccc54)c3)nc(-c3cccc4oc5cc(-c6cccc7oc8cc9ccccc9cc8c67)ccc5c34)n2)cc1 ; c1ccc(-c2nc(-c3ccc4c(ccc5ccccc54)c3)nc(-c3ccc(-c4cccc5sc6ccc7ccccc7c6c45)c4oc5ccccc5c34)n2)cc1 ; c1ccc(-c2nc(-c3ccc4c(ccc5ccccc54)c3)nc(-c3cccc4oc5cccc(-c6cccc7sc8ccccc8c67)c5c34)n2)cc1]

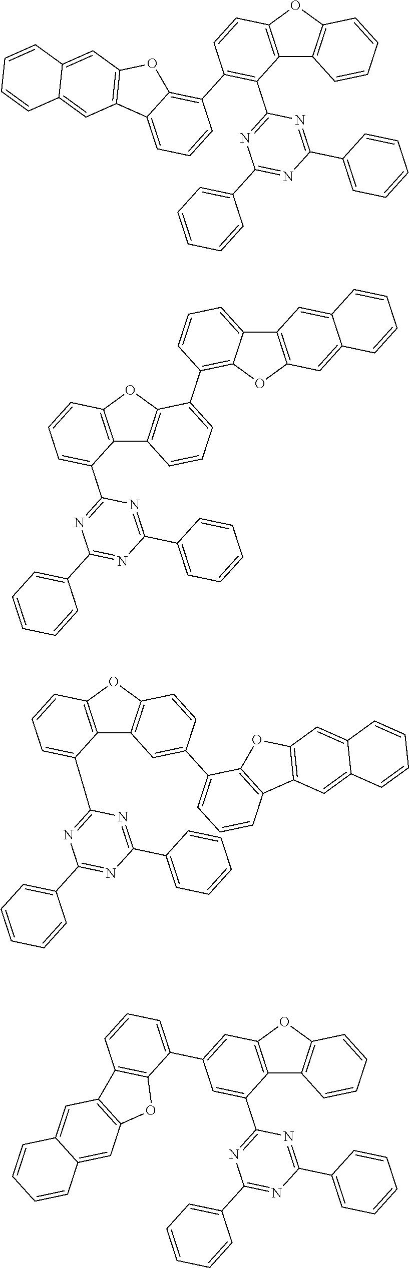 [SMILES: c1ccc(-c2nc(-c3ccccc3)nc(-c3c(-c4cccc5c4oc4cc6ccccc6cc45)ccc4oc5ccccc5c34)n2)cc1 ; c1ccc(-c2nc(-c3ccccc3)nc(-c3cccc4oc5c(-c6cccc7c6oc6cc8ccccc8cc67)cccc5c34)n2)cc1 ; c1ccc(-c2nc(-c3ccccc3)nc(-c3cccc4oc5ccc(-c6cccc7c6oc6cc8ccccc8cc67)cc5c34)n2)cc1 ; c1ccc(-c2nc(-c3ccccc3)nc(-c3cc(-c4cccc5c4oc4cc6ccccc6cc45)cc4oc5ccccc5c34)n2)cc1]

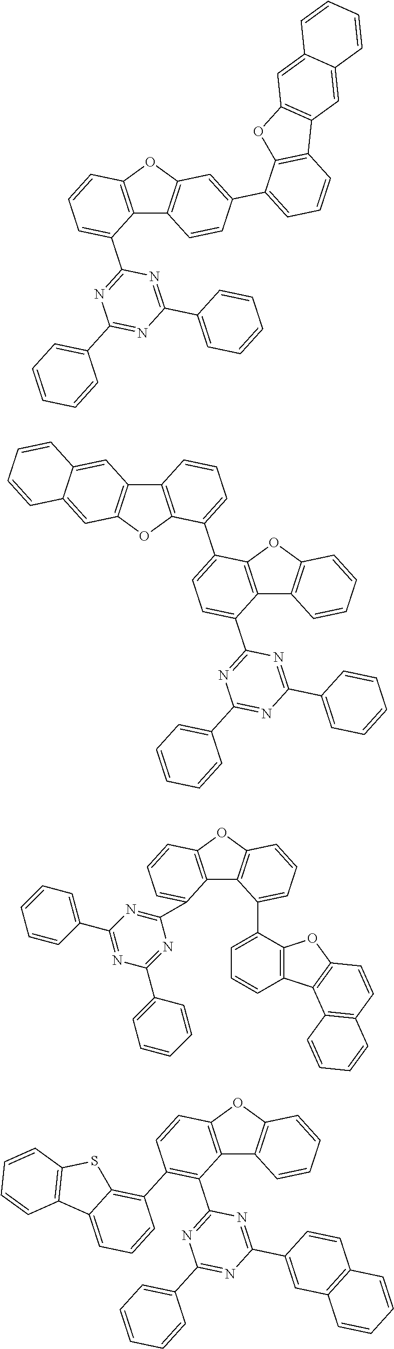

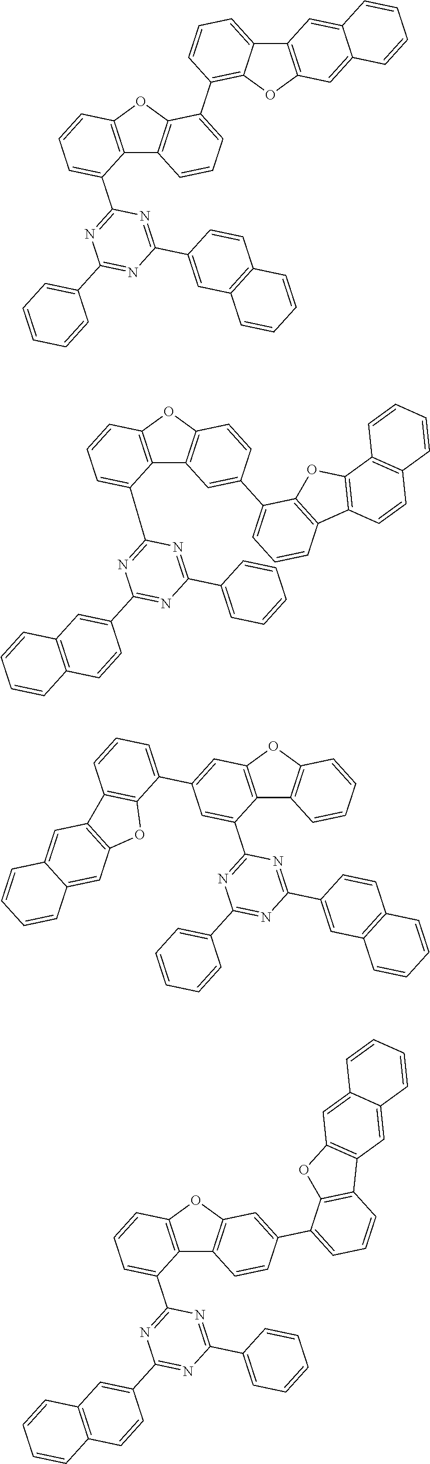 [SMILES: c1ccc(-c2nc(-c3ccc4ccccc4c3)nc(-c3cccc4oc5c(-c6cccc7c6oc6cc8ccccc8cc67)cccc5c34)n2)cc1 ; c1ccc(-c2nc(-c3ccc4ccccc4c3)nc(-c3cccc4oc5ccc(-c6cccc7c6oc6c8ccccc8ccc76)cc5c34)n2)cc1 ; c1ccc(-c2nc(-c3ccc4ccccc4c3)nc(-c3cc(-c4cccc5c4oc4cc6ccccc6cc45)cc4oc5ccccc5c34)n2)cc1 ; c1ccc(-c2nc(-c3ccc4ccccc4c3)nc(-c3cccc4oc5cc(-c6cccc7c6oc6cc8ccccc8cc67)ccc5c34)n2)cc1]

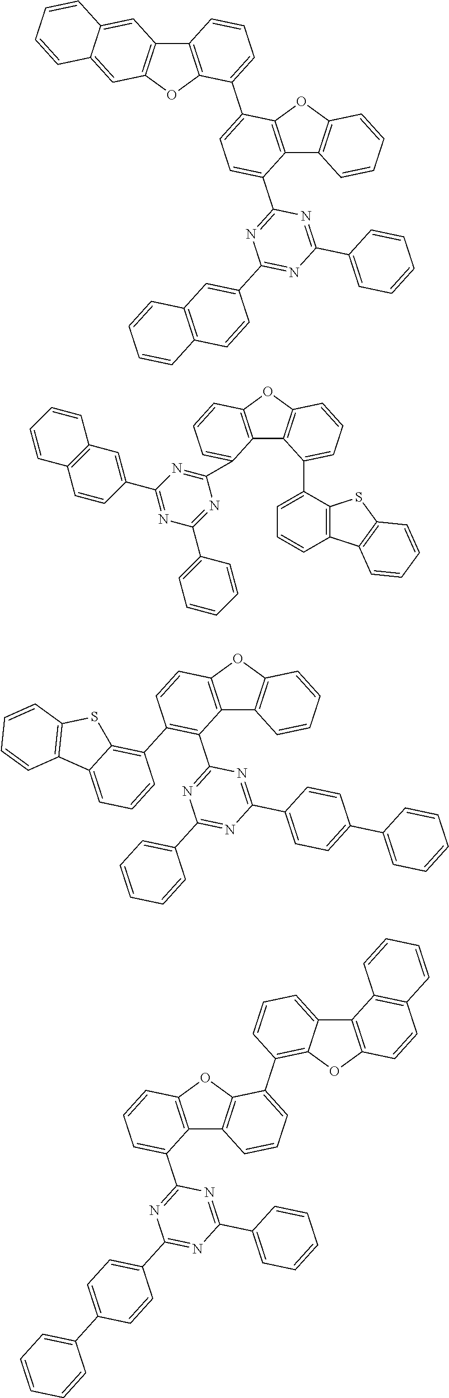 [SMILES: c1ccc(-c2nc(-c3ccc4ccccc4c3)nc(-c3ccc(-c4cccc5c4oc4cc6ccccc6cc45)c4oc5ccccc5c34)n2)cc1 ; c1ccc(-c2nc(-c3ccc4ccccc4c3)nc(-c3cccc4oc5cccc(-c6cccc7c6sc6ccccc67)c5c34)n2)cc1 ; c1ccc(-c2ccc(-c3nc(-c4ccccc4)nc(-c4c(-c5cccc6c5sc5ccccc56)ccc5oc6ccccc6c45)n3)cc2)cc1 ; c1ccc(-c2ccc(-c3nc(-c4ccccc4)nc(-c4cccc5oc6c(-c7cccc8c7oc7ccc9ccccc9c78)cccc6c45)n3)cc2)cc1]

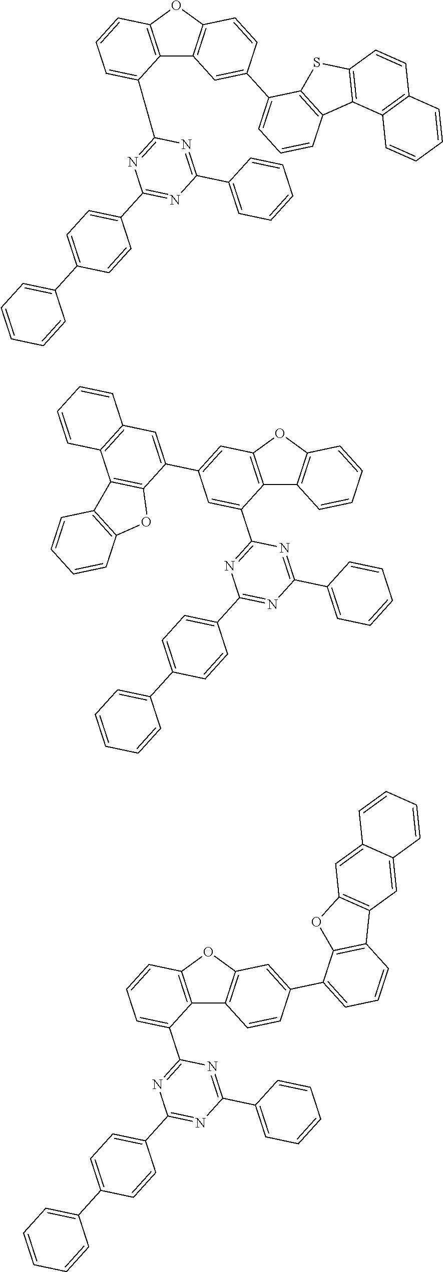 [SMILES: c1ccc(-c2ccc(-c3nc(-c4ccccc4)nc(-c4cccc5oc6ccc(-c7cccc8c7sc7ccc9ccccc9c78)cc6c45)n3)cc2)cc1 ; c1ccc(-c2ccc(-c3nc(-c4ccccc4)nc(-c4cc(-c5cc6ccccc6c6c5oc5ccccc56)cc5oc6ccccc6c45)n3)cc2)cc1 ; c1ccc(-c2ccc(-c3nc(-c4ccccc4)nc(-c4cccc5oc6cc(-c7cccc8c7oc7cc9ccccc9cc78)ccc6c45)n3)cc2)cc1]

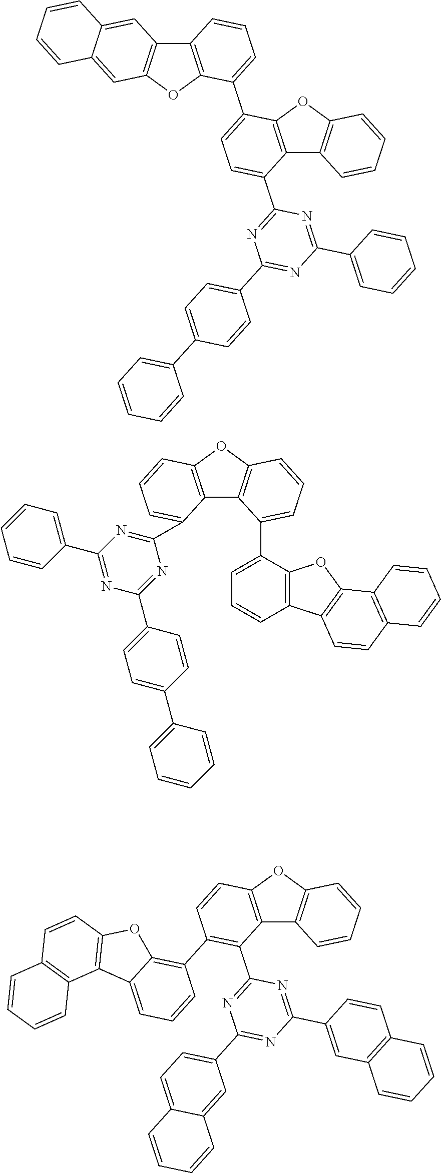

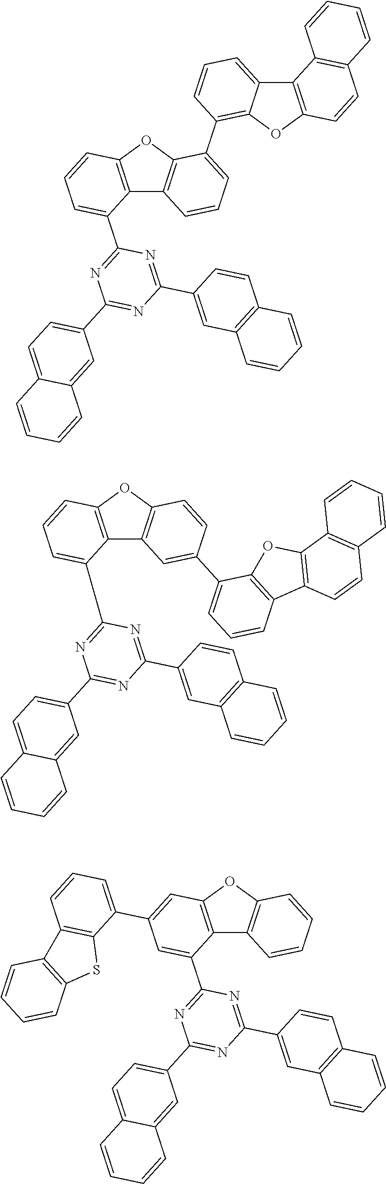 [SMILES: c1ccc2cc(-c3nc(-c4ccc5ccccc5c4)nc(-c4cccc5oc6c(-c7cccc8c7oc7ccc9ccccc9c78)cccc6c45)n3)ccc2c1 ; c1ccc2cc(-c3nc(-c4ccc5ccccc5c4)nc(-c4cccc5oc6ccc(-c7cccc8c7oc7c9ccccc9ccc87)cc6c45)n3)ccc2c1 ; c1ccc2cc(-c3nc(-c4ccc5ccccc5c4)nc(-c4cc(-c5cccc6c5sc5ccccc56)cc5oc6ccccc6c45)n3)ccc2c1]

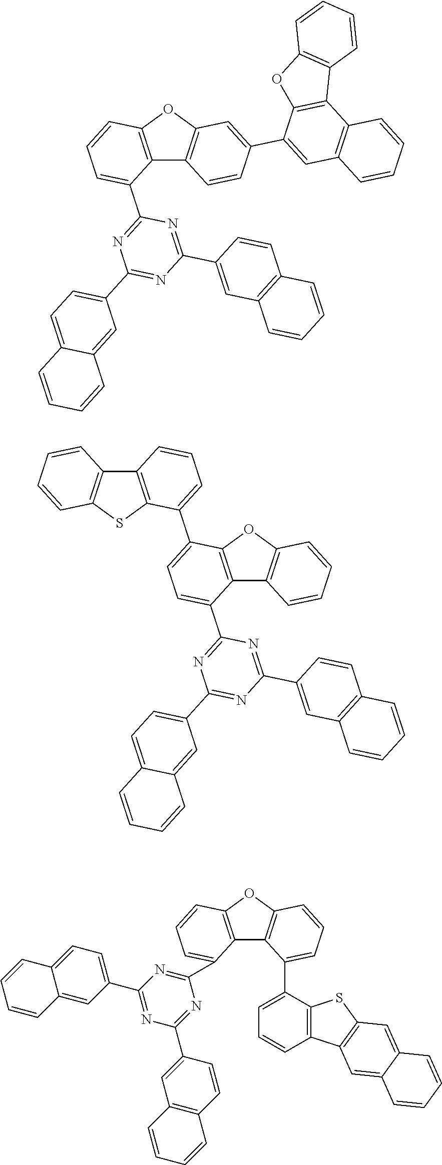

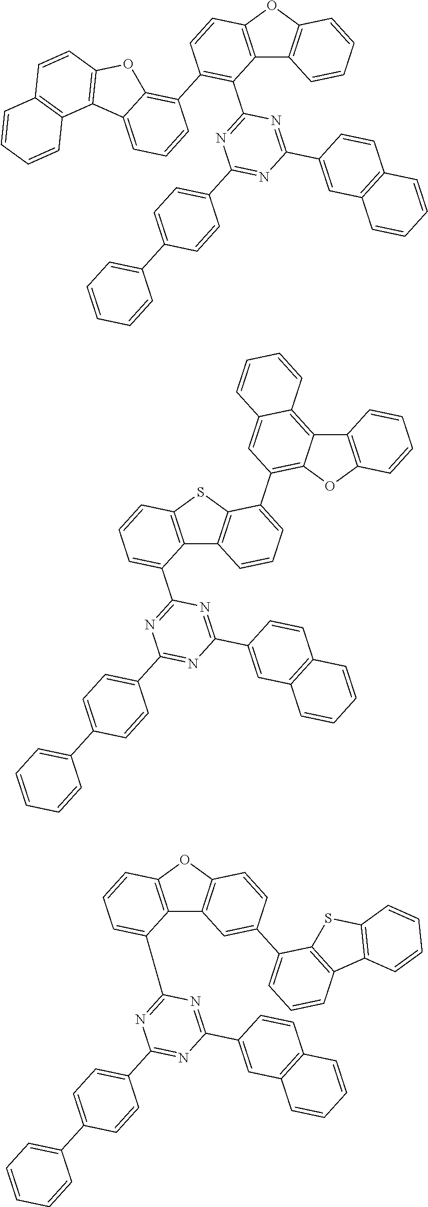 [SMILES: c1ccc(-c2ccc(-c3nc(-c4ccc5ccccc5c4)nc(-c4c(-c5cccc6c5oc5ccc7ccccc7c56)ccc5oc6ccccc6c45)n3)cc2)cc1 ; c1ccc(-c2ccc(-c3nc(-c4ccc5ccccc5c4)nc(-c4cccc5sc6c(-c7cc8ccccc8c8c7oc7ccccc78)cccc6c45)n3)cc2)cc1 ; c1ccc(-c2ccc(-c3nc(-c4ccc5ccccc5c4)nc(-c4cccc5oc6ccc(-c7cccc8c7sc7ccccc78)cc6c45)n3)cc2)cc1]

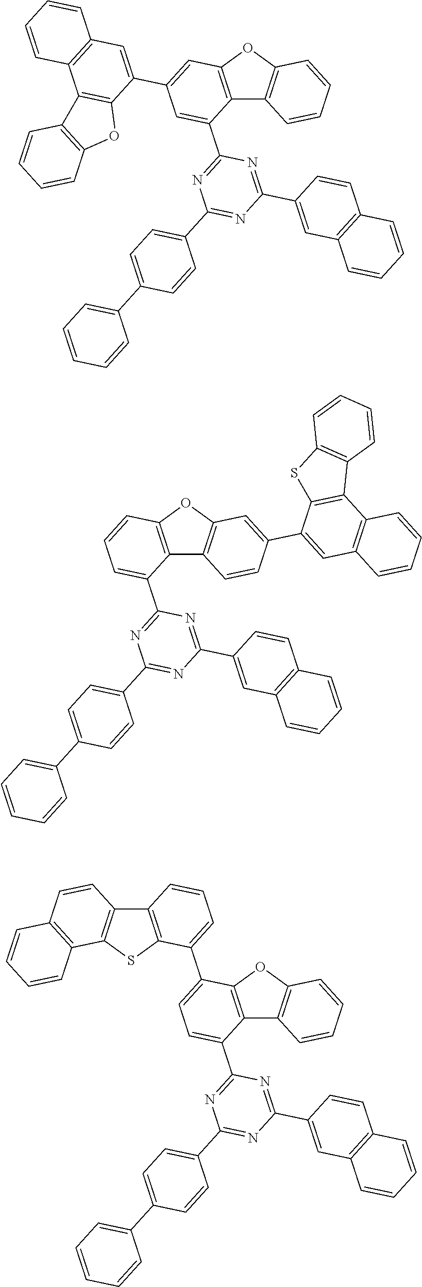 [SMILES: c1ccc(-c2ccc(-c3nc(-c4ccc5ccccc5c4)nc(-c4cc(-c5cc6ccccc6c6c5oc5ccccc56)cc5oc6ccccc6c45)n3)cc2)cc1 ; c1ccc(-c2ccc(-c3nc(-c4ccc5ccccc5c4)nc(-c4cccc5oc6cc(-c7cc8ccccc8c8c7sc7ccccc78)ccc6c45)n3)cc2)cc1 ; c1ccc(-c2ccc(-c3nc(-c4ccc5ccccc5c4)nc(-c4ccc(-c5cccc6c5sc5c7ccccc7ccc65)c5oc6ccccc6c45)n3)cc2)cc1]

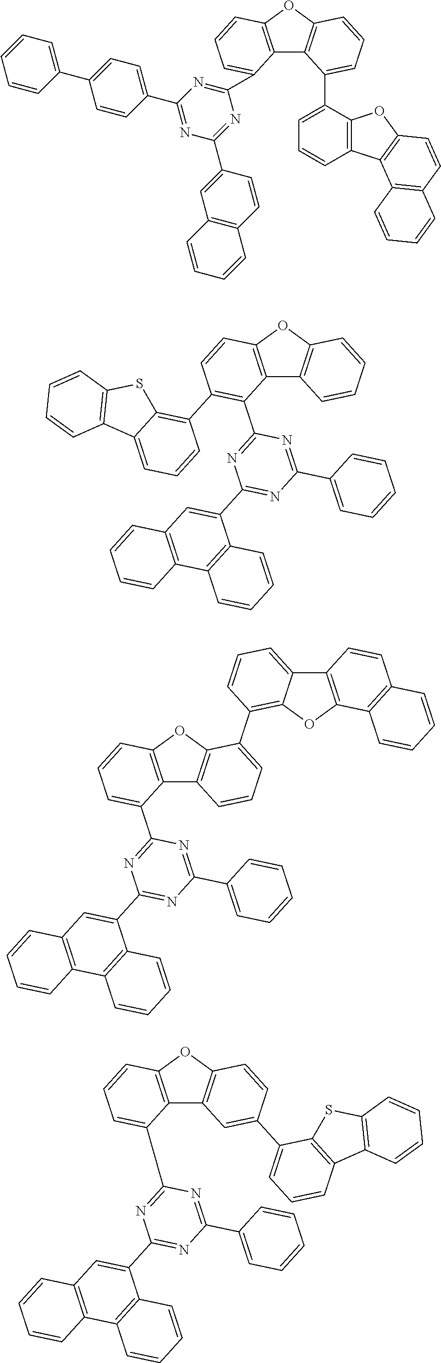

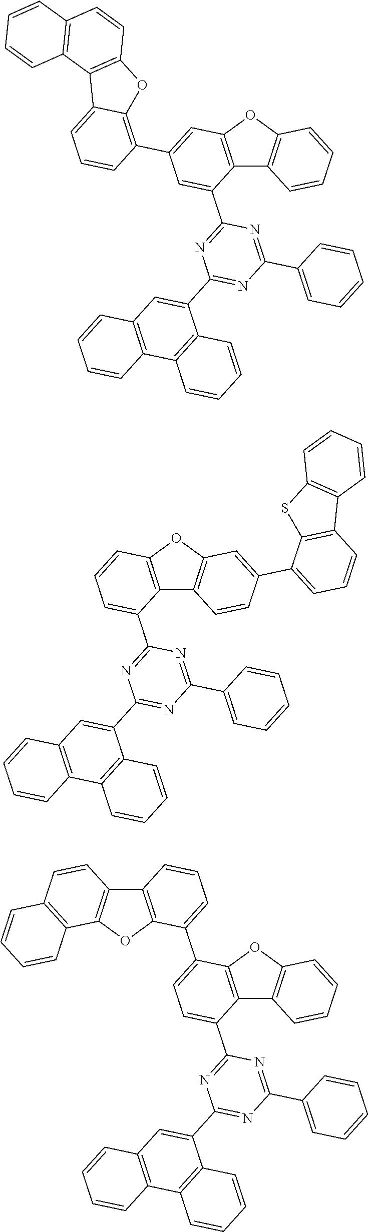 [SMILES: c1ccc(-c2nc(-c3cc4ccccc4c4ccccc34)nc(-c3cc(-c4cccc5c4oc4ccc6ccccc6c45)cc4oc5ccccc5c34)n2)cc1 ; c1ccc(-c2nc(-c3cc4ccccc4c4ccccc34)nc(-c3cccc4oc5cc(-c6cccc7c6sc6ccccc67)ccc5c34)n2)cc1 ; c1ccc(-c2nc(-c3cc4ccccc4c4ccccc34)nc(-c3ccc(-c4cccc5c4oc4c6ccccc6ccc54)c4oc5ccccc5c34)n2)cc1]

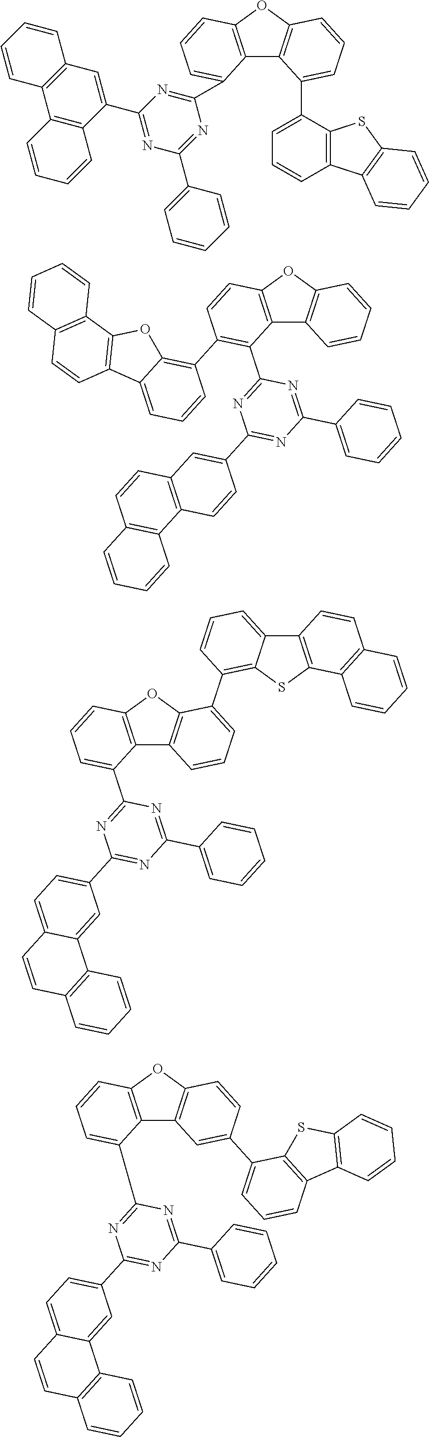

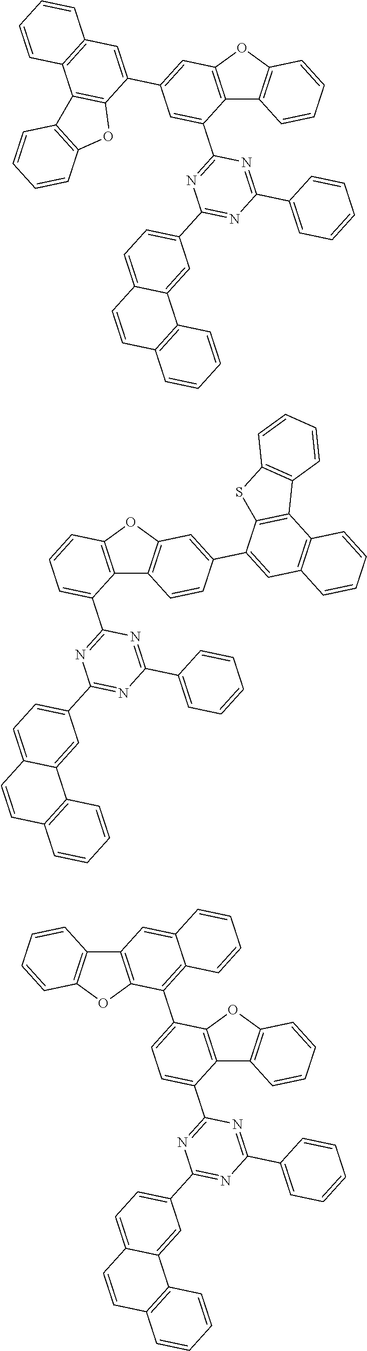 [SMILES: c1ccc(-c2nc(-c3ccc4ccc5ccccc5c4c3)nc(-c3cc(-c4cc5ccccc5c5c4oc4ccccc45)cc4oc5ccccc5c34)n2)cc1 ; c1ccc(-c2nc(-c3ccc4ccc5ccccc5c4c3)nc(-c3cccc4oc5cc(-c6cc7ccccc7c7c6sc6ccccc67)ccc5c34)n2)cc1 ; c1ccc(-c2nc(-c3ccc4ccc5ccccc5c4c3)nc(-c3ccc(-c4c5ccccc5cc5c4oc4ccccc45)c4oc5ccccc5c34)n2)cc1]

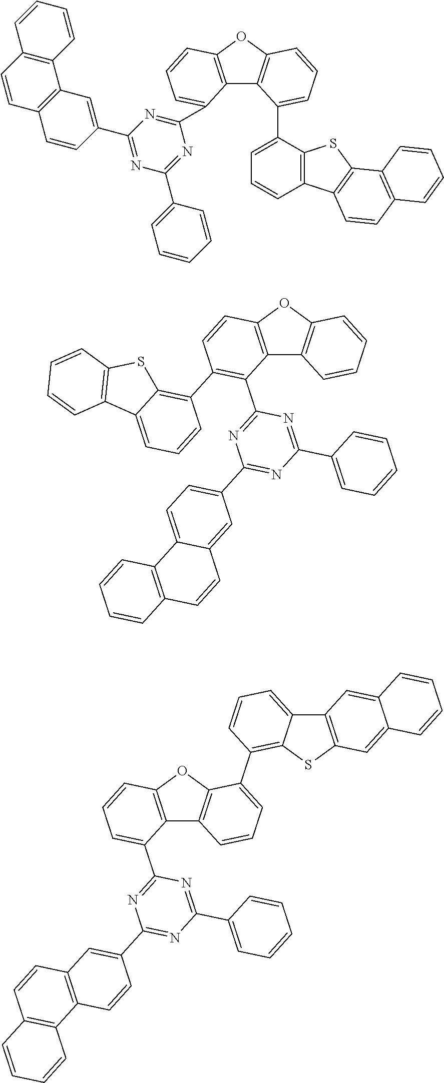 [SMILES: c1ccc(-c2nc(-c3ccc4ccc5ccccc5c4c3)nc(-c3cccc4oc5cccc(-c6cccc7c6sc6c8ccccc8ccc76)c5c34)n2)cc1 ; c1ccc(-c2nc(-c3ccc4c(ccc5ccccc54)c3)nc(-c3c(-c4cccc5c4sc4ccccc45)ccc4oc5ccccc5c34)n2)cc1 ; c1ccc(-c2nc(-c3ccc4c(ccc5ccccc54)c3)nc(-c3cccc4oc5c(-c6cccc7c6sc6cc8ccccc8cc67)cccc5c34)n2)cc1]

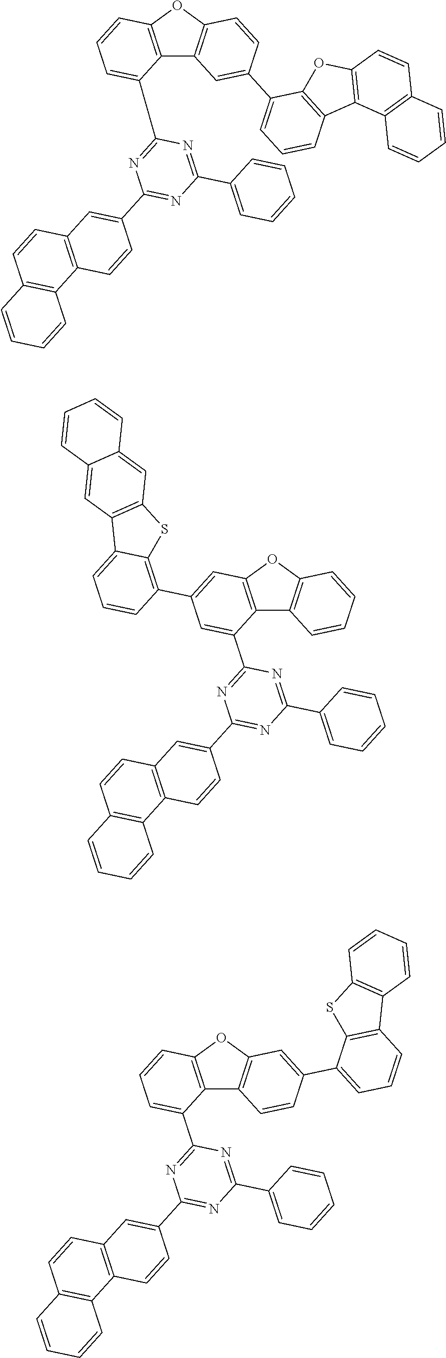 [SMILES: c1ccc(-c2nc(-c3ccc4c(ccc5ccccc54)c3)nc(-c3cccc4oc5ccc(-c6cccc7c6oc6ccc8ccccc8c67)cc5c34)n2)cc1 ; c1ccc(-c2nc(-c3ccc4c(ccc5ccccc54)c3)nc(-c3cc(-c4cccc5c4sc4cc6ccccc6cc45)cc4oc5ccccc5c34)n2)cc1 ; c1ccc(-c2nc(-c3ccc4c(ccc5ccccc54)c3)nc(-c3cccc4oc5cc(-c6cccc7c6sc6ccccc67)ccc5c34)n2)cc1]

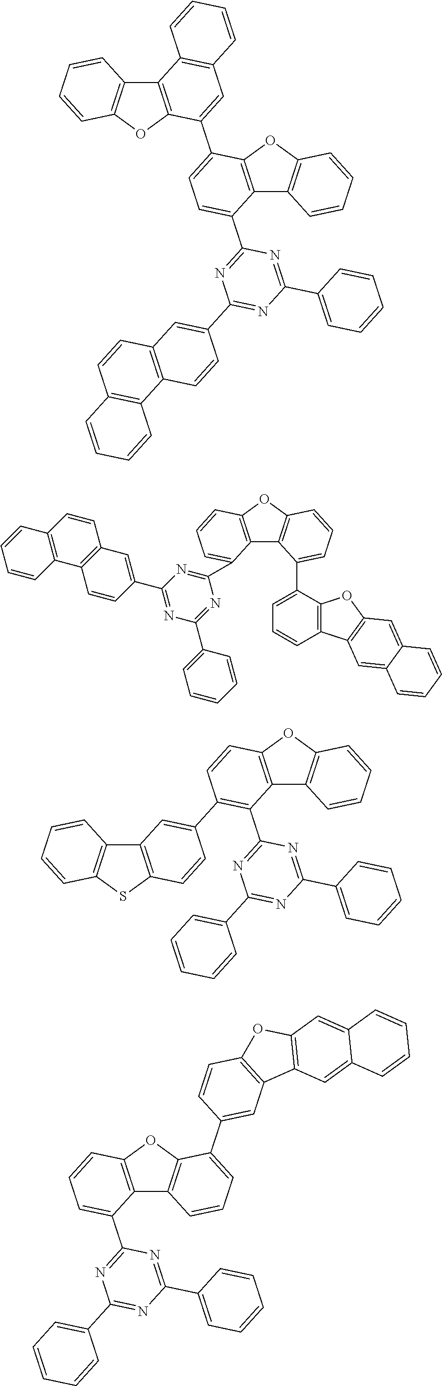

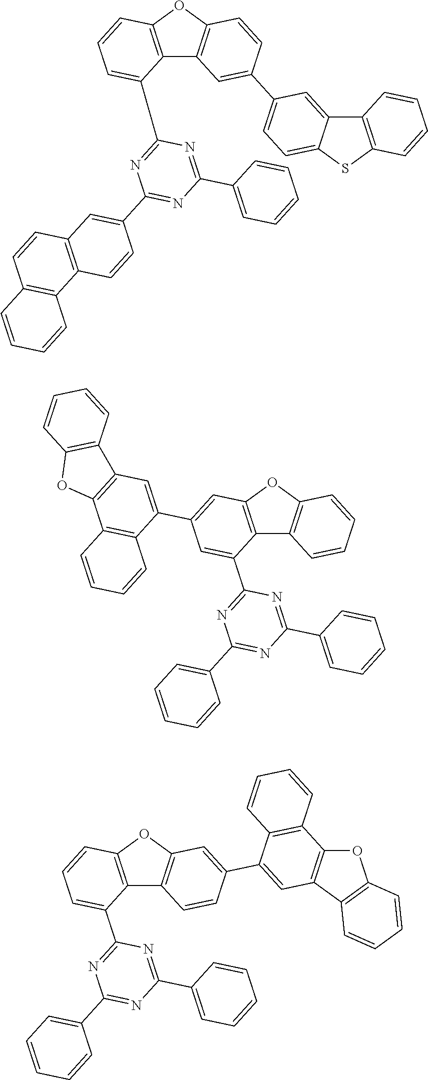

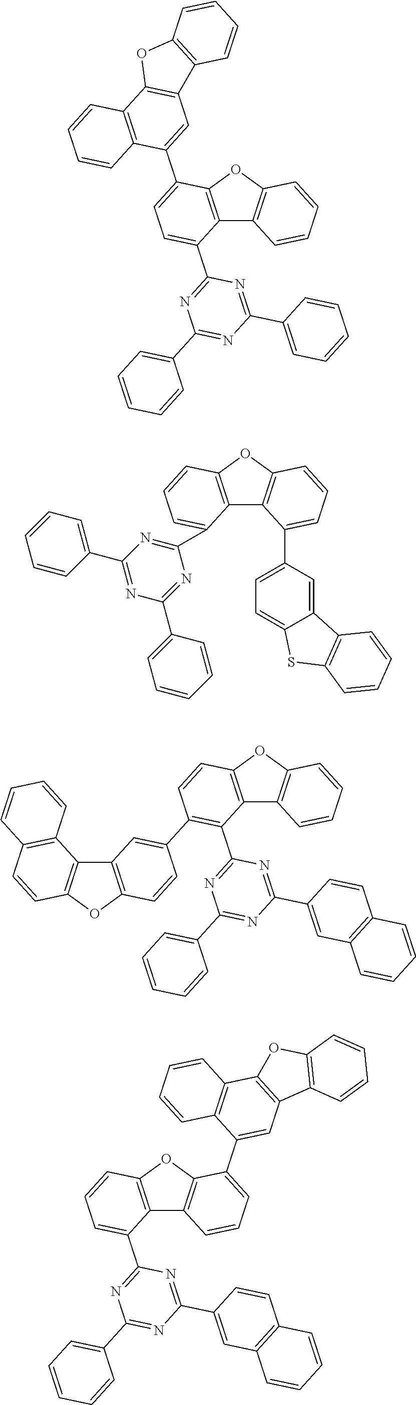 [SMILES: c1ccc(-c2nc(-c3ccccc3)nc(-c3ccc(-c4cc5c6ccccc6oc5c5ccccc45)c4oc5ccccc5c34)n2)cc1 ; c1ccc(-c2nc(-c3ccccc3)nc(-c3cccc4oc5cccc(-c6ccc7sc8ccccc8c7c6)c5c34)n2)cc1 ; c1ccc(-c2nc(-c3ccc4ccccc4c3)nc(-c3c(-c4ccc5oc6ccc7ccccc7c6c5c4)ccc4oc5ccccc5c34)n2)cc1 ; c1ccc(-c2nc(-c3ccc4ccccc4c3)nc(-c3cccc4oc5c(-c6cc7c8ccccc8oc7c7ccccc67)cccc5c34)n2)cc1]

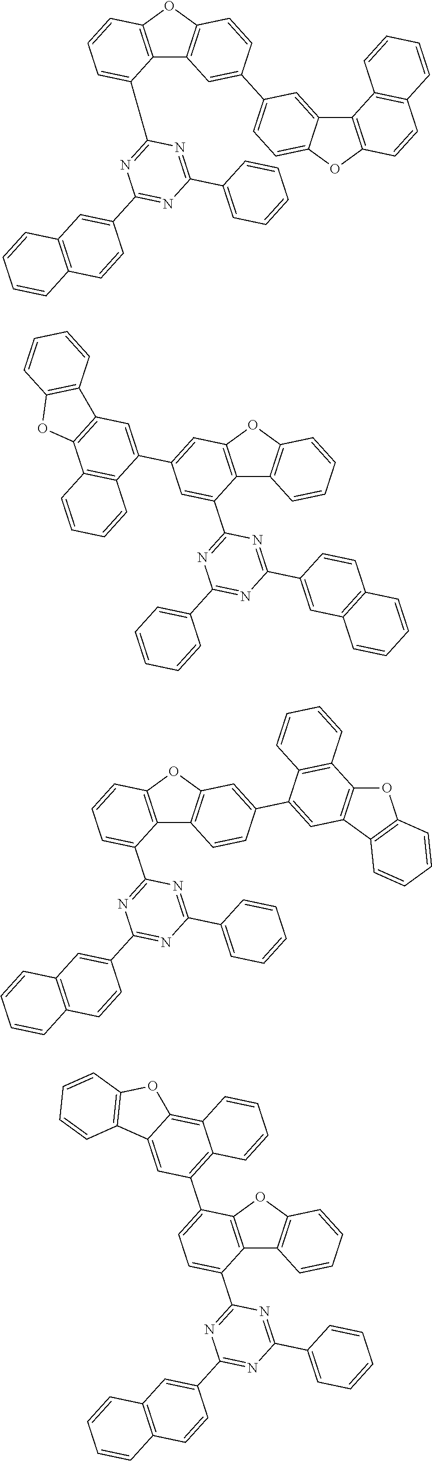

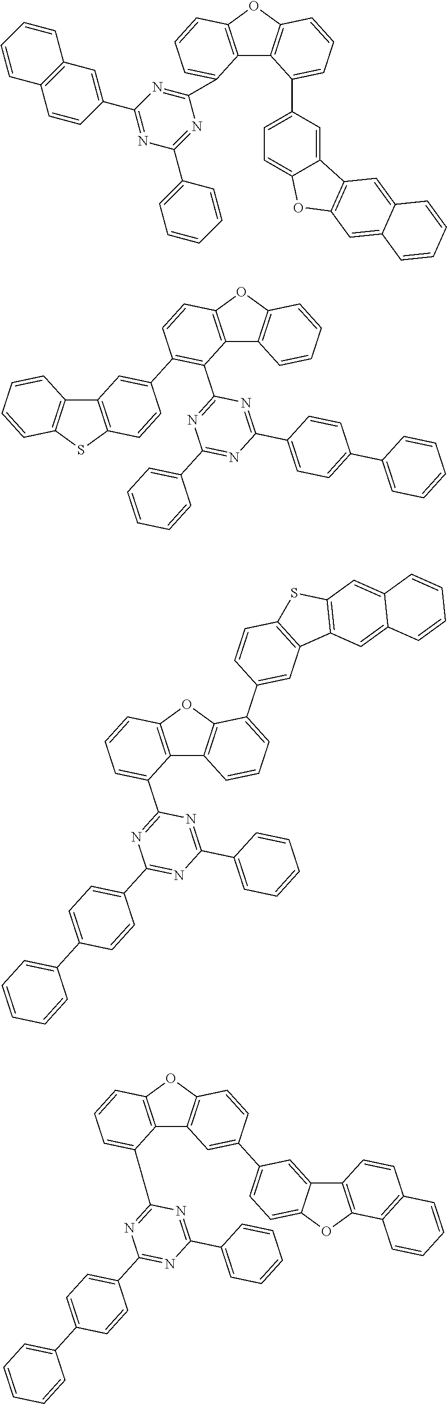 [SMILES: c1ccc(-c2nc(-c3ccc4ccccc4c3)nc(-c3cccc4oc5cccc(-c6ccc7oc8cc9ccccc9cc8c7c6)c5c34)n2)cc1 ; c1ccc(-c2ccc(-c3nc(-c4ccccc4)nc(-c4c(-c5ccc6sc7ccccc7c6c5)ccc5oc6ccccc6c45)n3)cc2)cc1 ; c1ccc(-c2ccc(-c3nc(-c4ccccc4)nc(-c4cccc5oc6c(-c7ccc8sc9cc%10ccccc%10cc9c8c7)cccc6c45)n3)cc2)cc1 ; c1ccc(-c2ccc(-c3nc(-c4ccccc4)nc(-c4cccc5oc6ccc(-c7ccc8oc9c%10ccccc%10ccc9c8c7)cc6c45)n3)cc2)cc1]

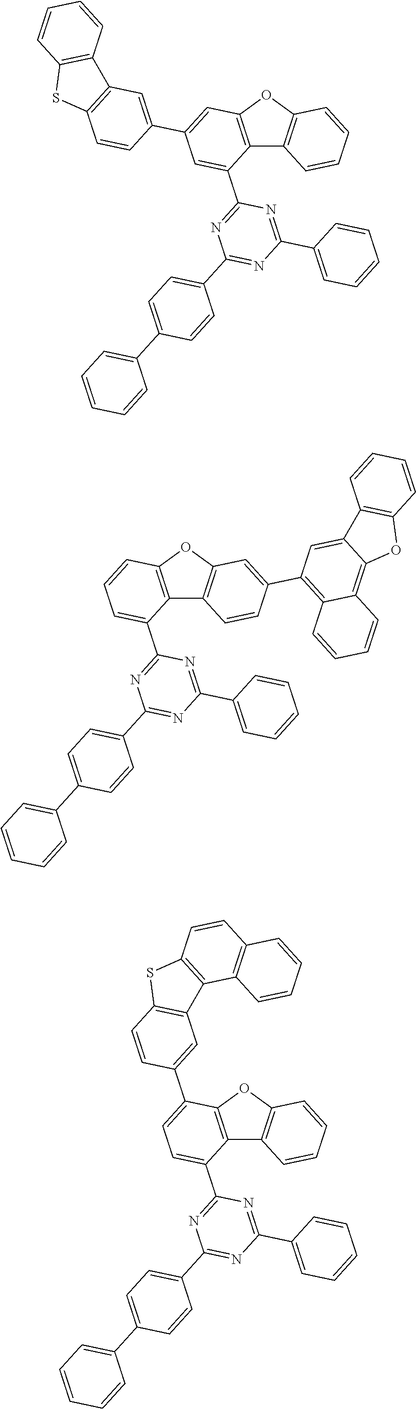 [SMILES: c1ccc(-c2ccc(-c3nc(-c4ccccc4)nc(-c4cc(-c5ccc6sc7ccccc7c6c5)cc5oc6ccccc6c45)n3)cc2)cc1 ; c1ccc(-c2ccc(-c3nc(-c4ccccc4)nc(-c4cccc5oc6cc(-c7cc8c9ccccc9oc8c8ccccc78)ccc6c45)n3)cc2)cc1 ; c1ccc(-c2ccc(-c3nc(-c4ccccc4)nc(-c4ccc(-c5ccc6sc7ccc8ccccc8c7c6c5)c5oc6ccccc6c45)n3)cc2)cc1]

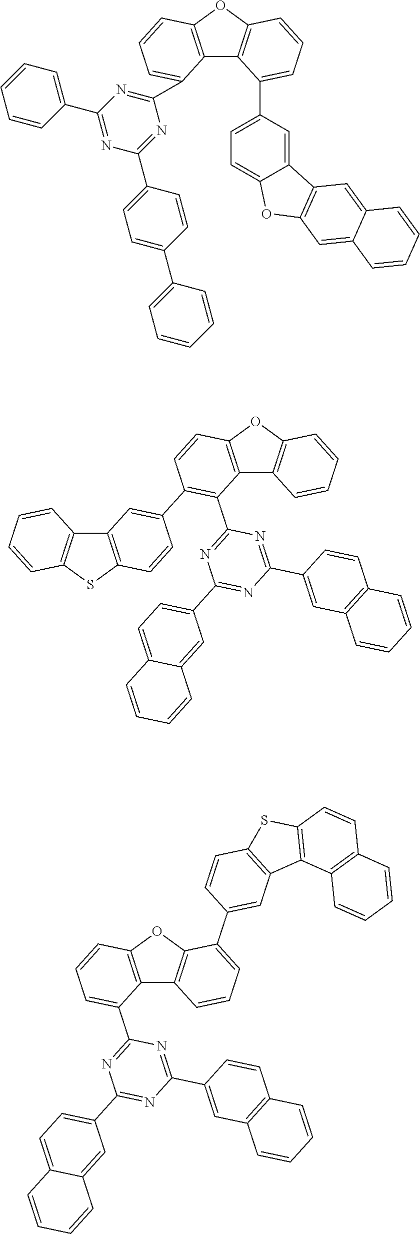 [SMILES: c1ccc(-c2ccc(-c3nc(-c4ccccc4)nc(-c4cccc5oc6cccc(-c7ccc8oc9cc%10ccccc%10cc9c8c7)c6c45)n3)cc2)cc1 ; c1ccc2cc(-c3nc(-c4ccc5ccccc5c4)nc(-c4c(-c5ccc6sc7ccccc7c6c5)ccc5oc6ccccc6c45)n3)ccc2c1 ; c1ccc2cc(-c3nc(-c4ccc5ccccc5c4)nc(-c4cccc5oc6c(-c7ccc8sc9ccc%10ccccc%10c9c8c7)cccc6c45)n3)ccc2c1]

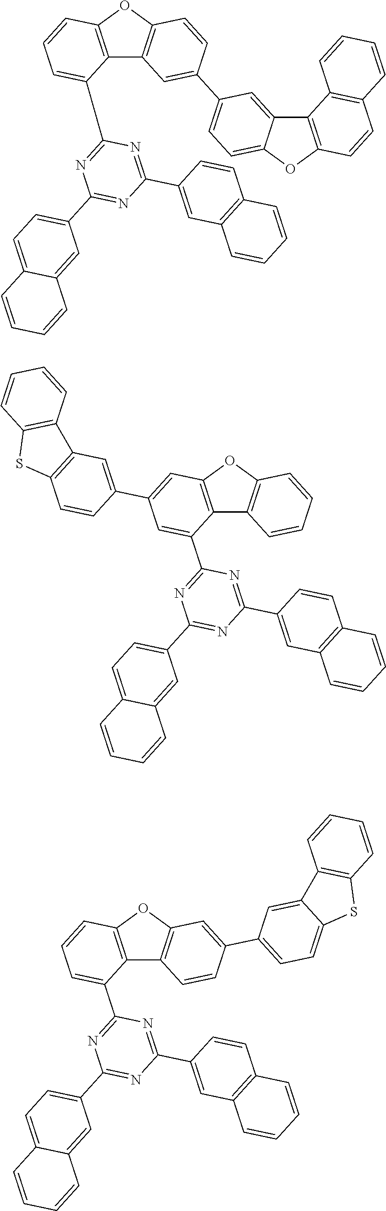

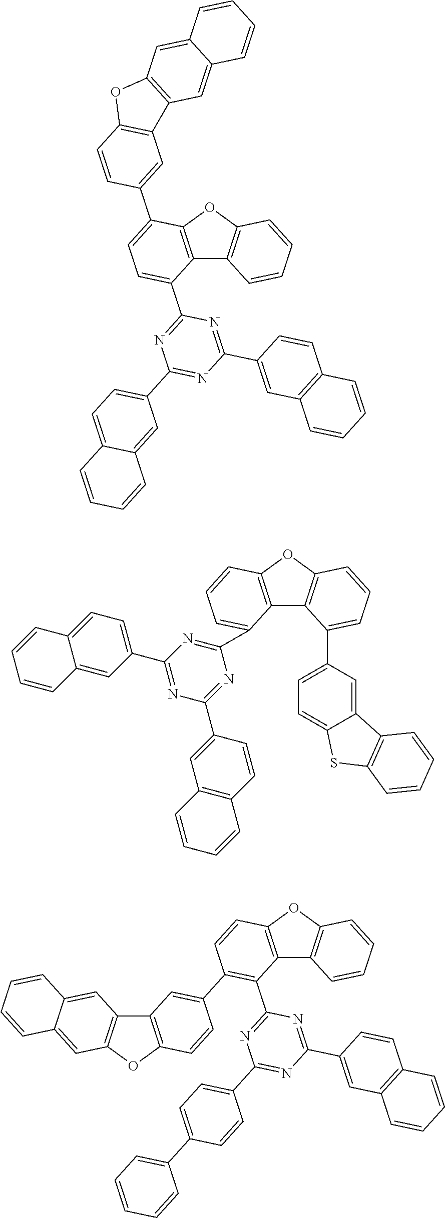 [SMILES: c1ccc2cc(-c3nc(-c4ccc5ccccc5c4)nc(-c4ccc(-c5ccc6oc7cc8ccccc8cc7c6c5)c5oc6ccccc6c45)n3)ccc2c1 ; c1ccc2cc(-c3nc(-c4ccc5ccccc5c4)nc(-c4cccc5oc6cccc(-c7ccc8sc9ccccc9c8c7)c6c45)n3)ccc2c1 ; c1ccc(-c2ccc(-c3nc(-c4ccc5ccccc5c4)nc(-c4c(-c5ccc6oc7cc8ccccc8cc7c6c5)ccc5oc6ccccc6c45)n3)cc2)cc1]

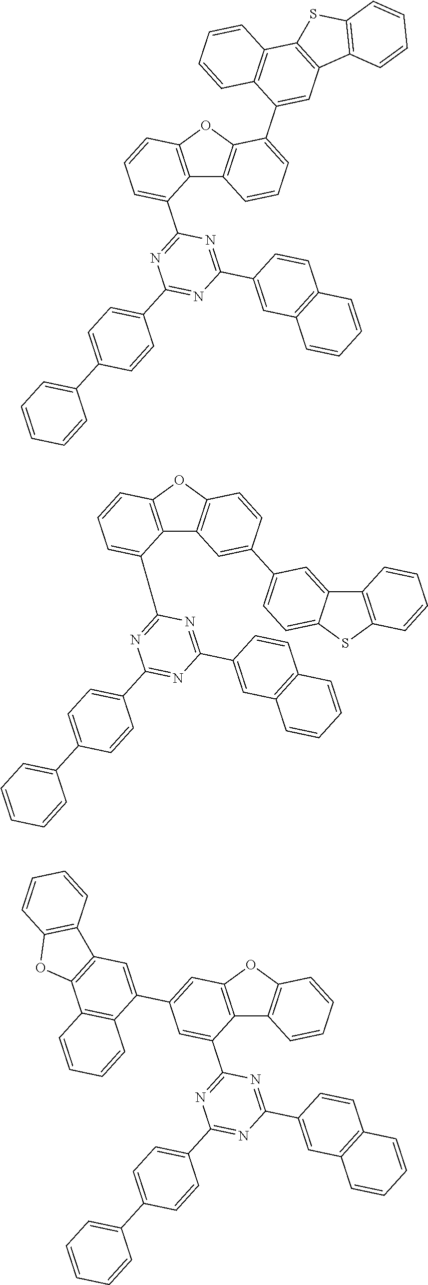 [SMILES: c1ccc(-c2ccc(-c3nc(-c4ccc5ccccc5c4)nc(-c4cccc5oc6c(-c7cc8c9ccccc9sc8c8ccccc78)cccc6c45)n3)cc2)cc1 ; c1ccc(-c2ccc(-c3nc(-c4ccc5ccccc5c4)nc(-c4cccc5oc6ccc(-c7ccc8sc9ccccc9c8c7)cc6c45)n3)cc2)cc1 ; c1ccc(-c2ccc(-c3nc(-c4ccc5ccccc5c4)nc(-c4cc(-c5cc6c7ccccc7oc6c6ccccc56)cc5oc6ccccc6c45)n3)cc2)cc1]

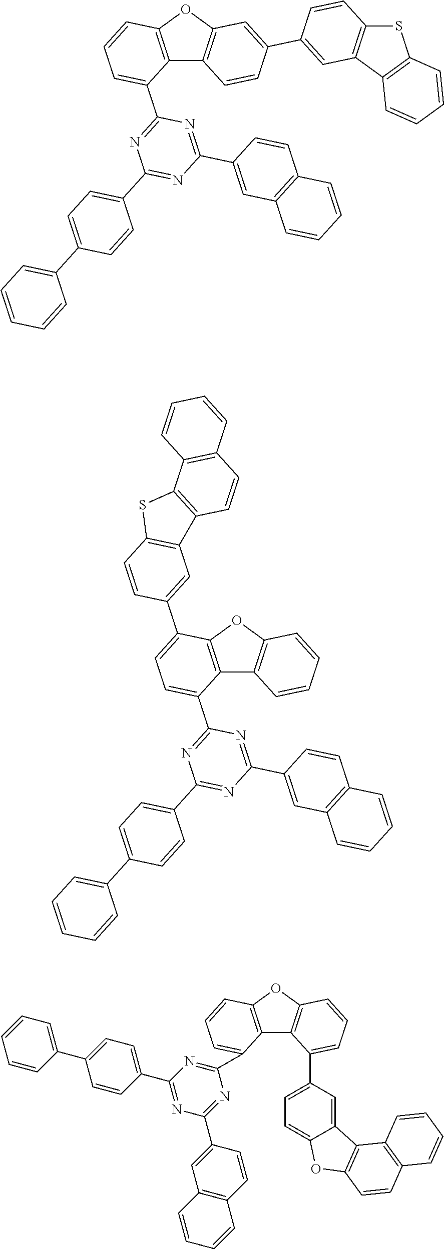 [SMILES: c1ccc(-c2ccc(-c3nc(-c4ccc5ccccc5c4)nc(-c4cccc5oc6cc(-c7ccc8sc9ccccc9c8c7)ccc6c45)n3)cc2)cc1 ; c1ccc(-c2ccc(-c3nc(-c4ccc5ccccc5c4)nc(-c4ccc(-c5ccc6sc7c8ccccc8ccc7c6c5)c5oc6ccccc6c45)n3)cc2)cc1 ; c1ccc(-c2ccc(-c3nc(-c4ccc5ccccc5c4)nc(-c4cccc5oc6cccc(-c7ccc8oc9ccc%10ccccc%10c9c8c7)c6c45)n3)cc2)cc1]

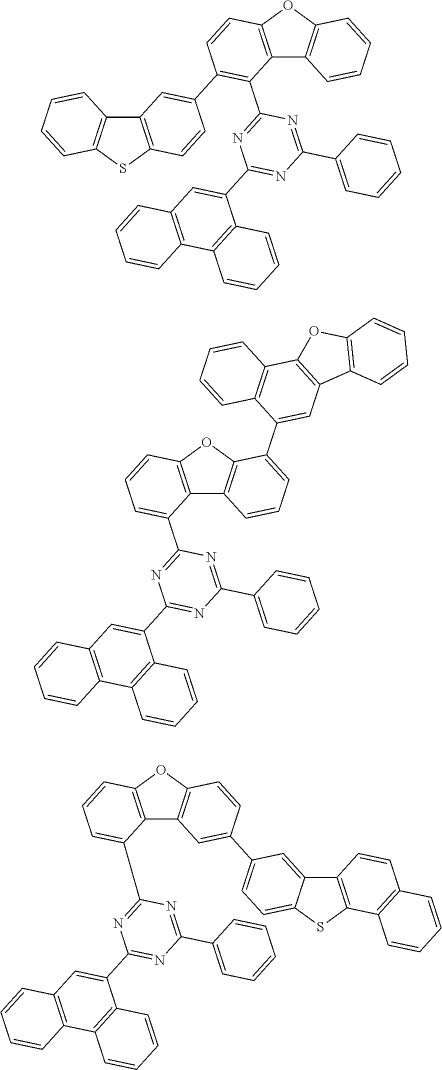

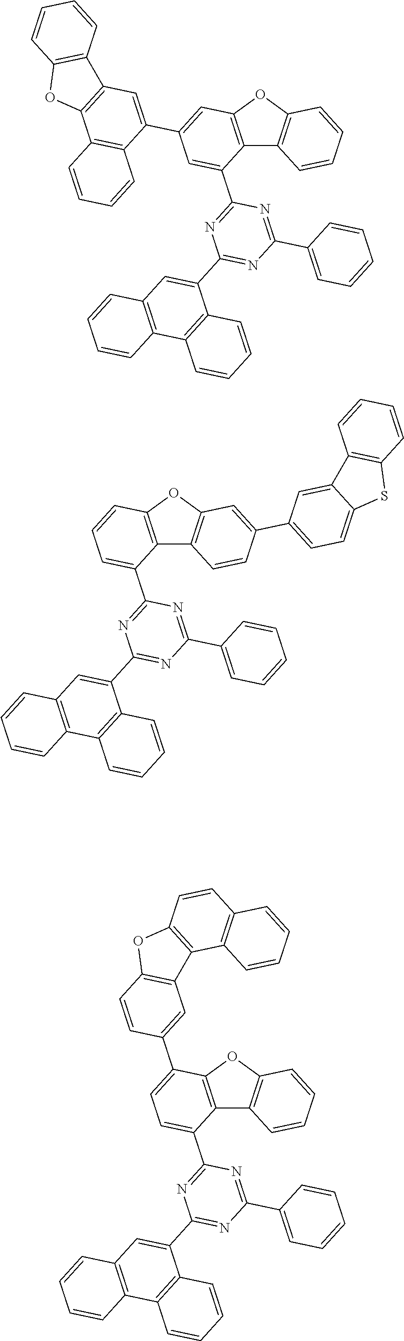 [SMILES: c1ccc(-c2nc(-c3cc4ccccc4c4ccccc34)nc(-c3cc(-c4cc5c6ccccc6oc5c5ccccc45)cc4oc5ccccc5c34)n2)cc1 ; c1ccc(-c2nc(-c3cc4ccccc4c4ccccc34)nc(-c3cccc4oc5cc(-c6ccc7sc8ccccc8c7c6)ccc5c34)n2)cc1 ; c1ccc(-c2nc(-c3cc4ccccc4c4ccccc34)nc(-c3ccc(-c4ccc5oc6ccc7ccccc7c6c5c4)c4oc5ccccc5c34)n2)cc1]

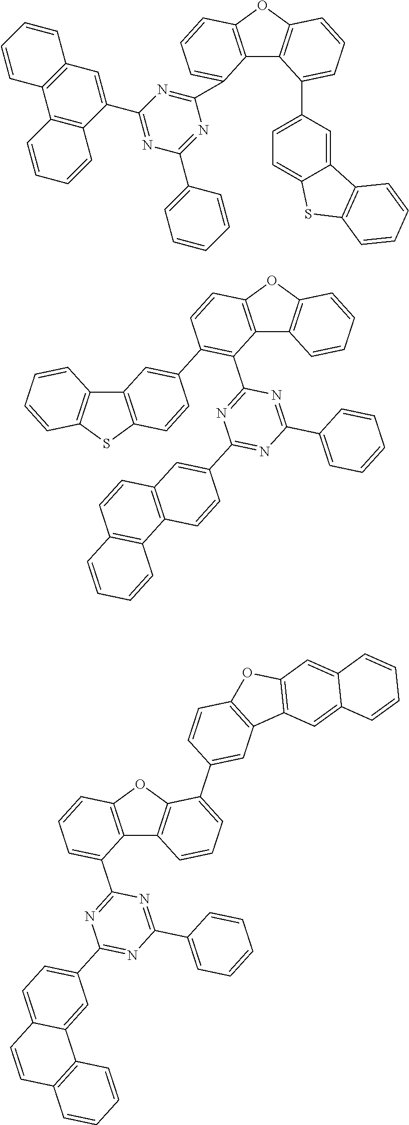

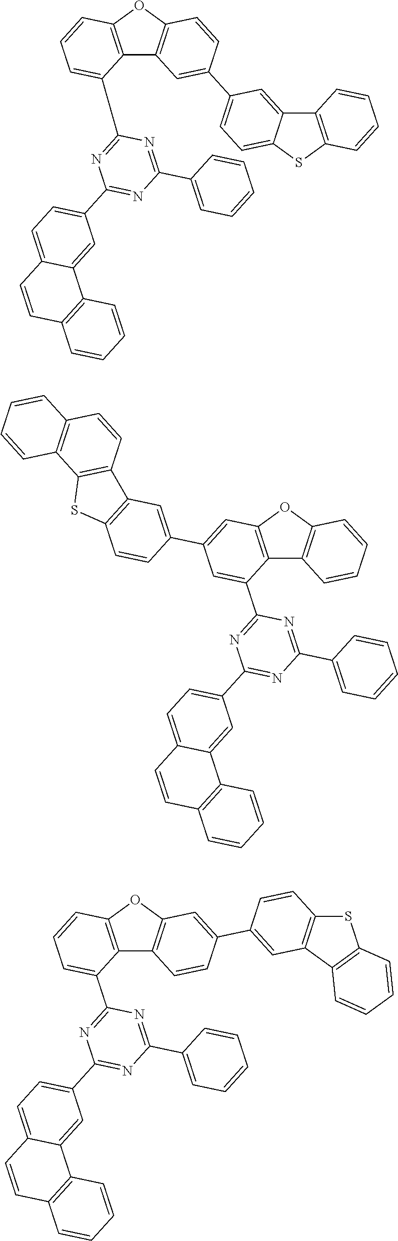 [SMILES: c1ccc(-c2nc(-c3ccc4ccc5ccccc5c4c3)nc(-c3cccc4oc5ccc(-c6ccc7sc8ccccc8c7c6)cc5c34)n2)cc1 ; c1ccc(-c2nc(-c3ccc4ccc5ccccc5c4c3)nc(-c3cc(-c4ccc5sc6c7ccccc7ccc6c5c4)cc4oc5ccccc5c34)n2)cc1 ; c1ccc(-c2nc(-c3ccc4ccc5ccccc5c4c3)nc(-c3cccc4oc5cc(-c6ccc7sc8ccccc8c7c6)ccc5c34)n2)cc1]

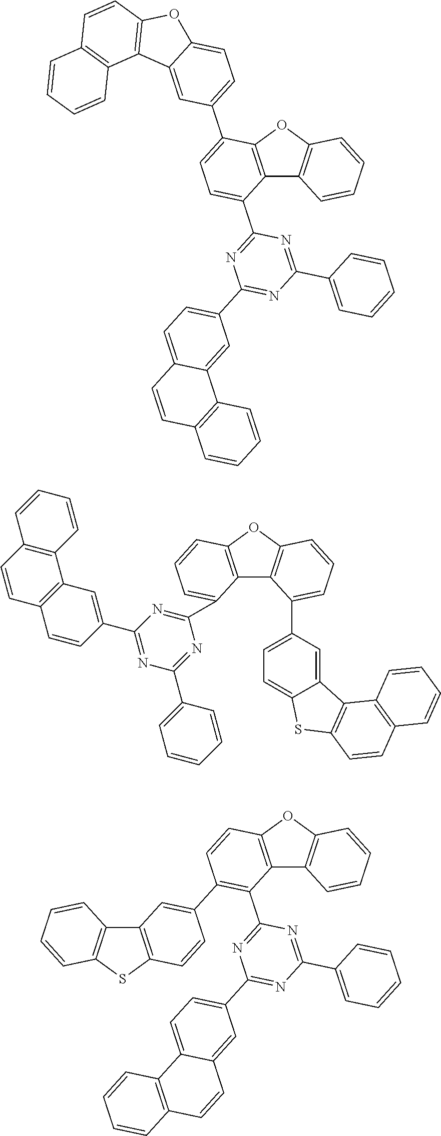 [SMILES: c1ccc(-c2nc(-c3ccc4ccc5ccccc5c4c3)nc(-c3ccc(-c4ccc5oc6ccc7ccccc7c6c5c4)c4oc5ccccc5c34)n2)cc1 ; c1ccc(-c2nc(-c3ccc4ccc5ccccc5c4c3)nc(-c3cccc4oc5cccc(-c6ccc7sc8ccc9ccccc9c8c7c6)c5c34)n2)cc1 ; c1ccc(-c2nc(-c3ccc4c(ccc5ccccc54)c3)nc(-c3c(-c4ccc5sc6ccccc6c5c4)ccc4oc5ccccc5c34)n2)cc1]

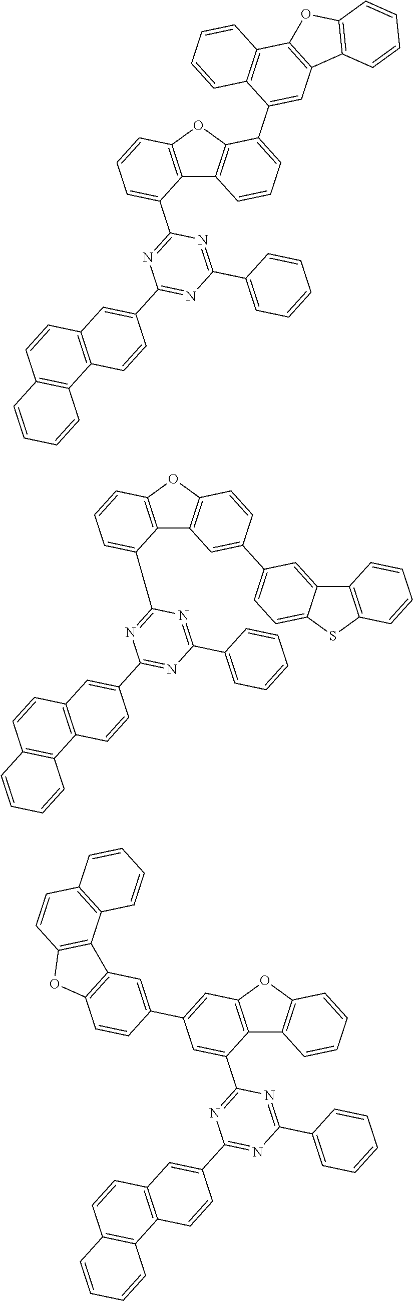 [SMILES: c1ccc(-c2nc(-c3ccc4c(ccc5ccccc54)c3)nc(-c3cccc4oc5c(-c6cc7c8ccccc8oc7c7ccccc67)cccc5c34)n2)cc1 ; c1ccc(-c2nc(-c3ccc4c(ccc5ccccc54)c3)nc(-c3cccc4oc5ccc(-c6ccc7sc8ccccc8c7c6)cc5c34)n2)cc1 ; c1ccc(-c2nc(-c3ccc4c(ccc5ccccc54)c3)nc(-c3cc(-c4ccc5oc6ccc7ccccc7c6c5c4)cc4oc5ccccc5c34)n2)cc1]

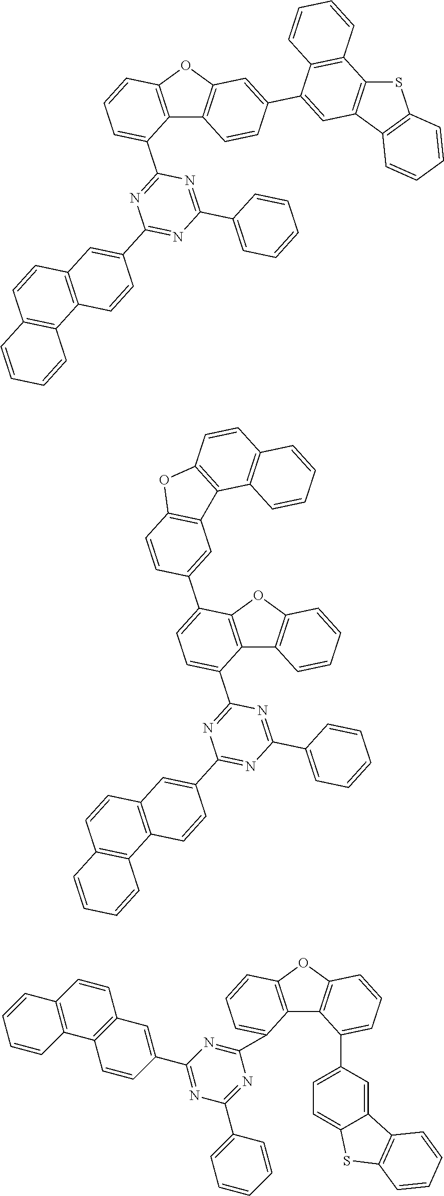 [SMILES: c1ccc(-c2nc(-c3ccc4c(ccc5ccccc54)c3)nc(-c3cccc4oc5cc(-c6cc7c8ccccc8sc7c7ccccc67)ccc5c34)n2)cc1 ; c1ccc(-c2nc(-c3ccc4c(ccc5ccccc54)c3)nc(-c3ccc(-c4ccc5oc6ccc7ccccc7c6c5c4)c4oc5ccccc5c34)n2)cc1 ; c1ccc(-c2nc(-c3ccc4c(ccc5ccccc54)c3)nc(-c3cccc4oc5cccc(-c6ccc7sc8ccccc8c7c6)c5c34)n2)cc1]

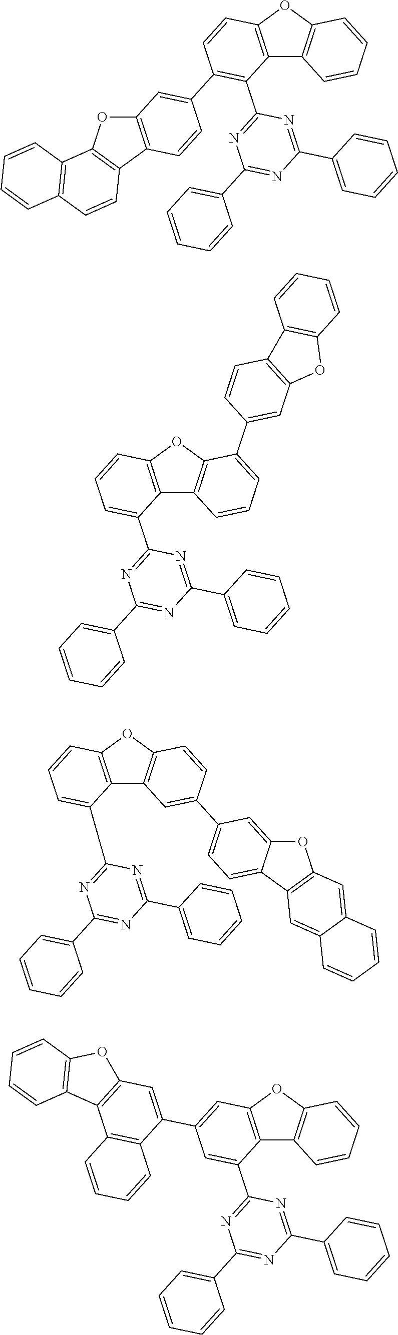

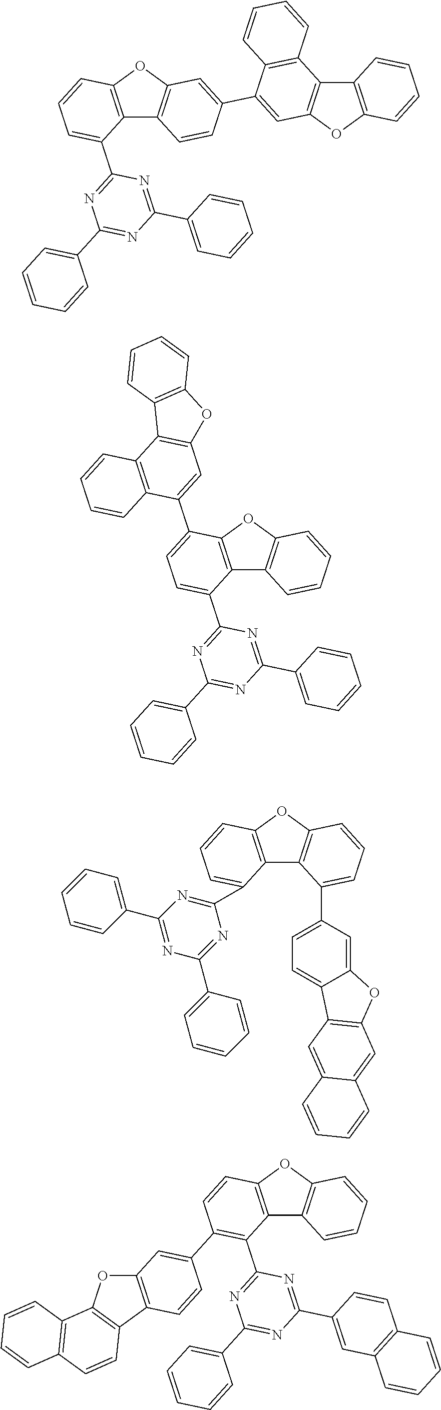

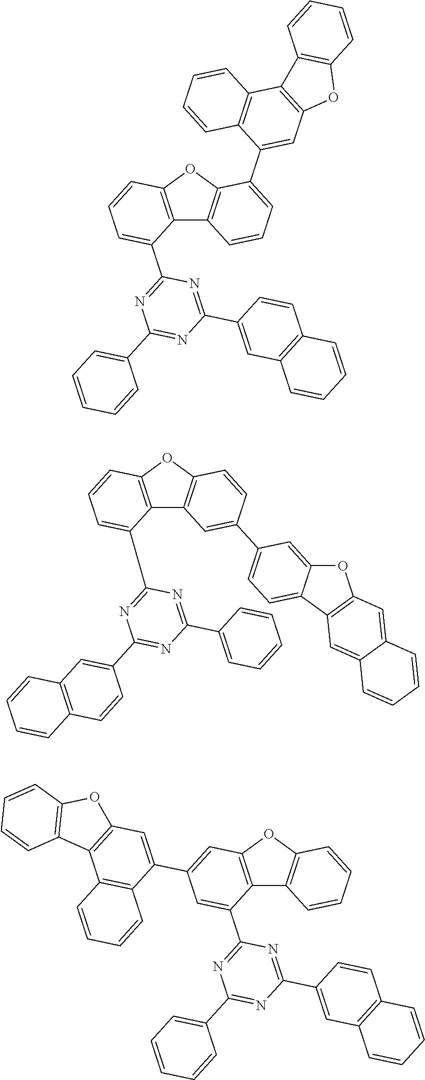 [SMILES: c1ccc(-c2nc(-c3ccc4ccccc4c3)nc(-c3cccc4oc5c(-c6cc7oc8ccccc8c7c7ccccc67)cccc5c34)n2)cc1 ; c1ccc(-c2nc(-c3ccc4ccccc4c3)nc(-c3cccc4oc5ccc(-c6ccc7c(c6)oc6cc8ccccc8cc67)cc5c34)n2)cc1 ; c1ccc(-c2nc(-c3ccc4ccccc4c3)nc(-c3cc(-c4cc5oc6ccccc6c5c5ccccc45)cc4oc5ccccc5c34)n2)cc1]

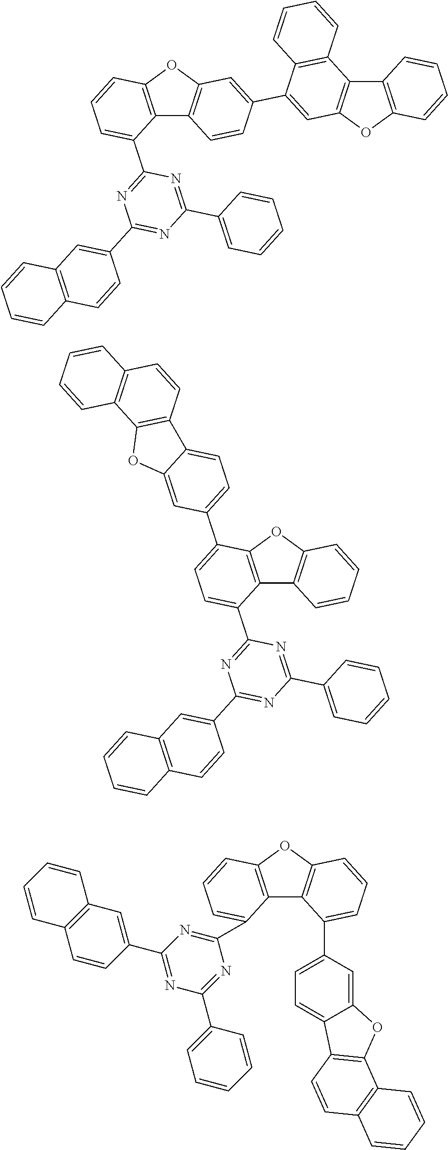

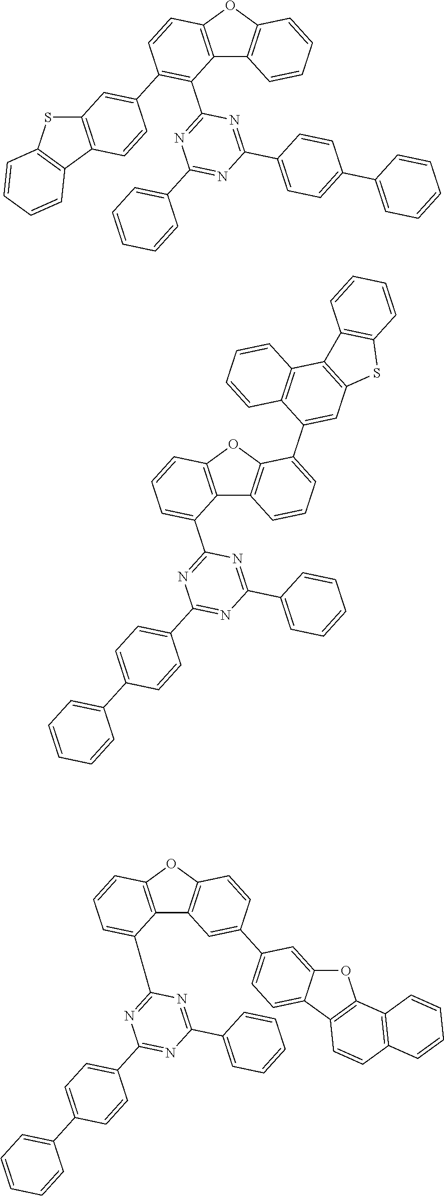 [SMILES: c1ccc(-c2ccc(-c3nc(-c4ccccc4)nc(-c4c(-c5ccc6c(c5)sc5ccccc56)ccc5oc6ccccc6c45)n3)cc2)cc1 ; c1ccc(-c2ccc(-c3nc(-c4ccccc4)nc(-c4cccc5oc6c(-c7cc8sc9ccccc9c8c8ccccc78)cccc6c45)n3)cc2)cc1 ; c1ccc(-c2ccc(-c3nc(-c4ccccc4)nc(-c4cccc5oc6ccc(-c7ccc8c(c7)oc7c9ccccc9ccc87)cc6c45)n3)cc2)cc1]

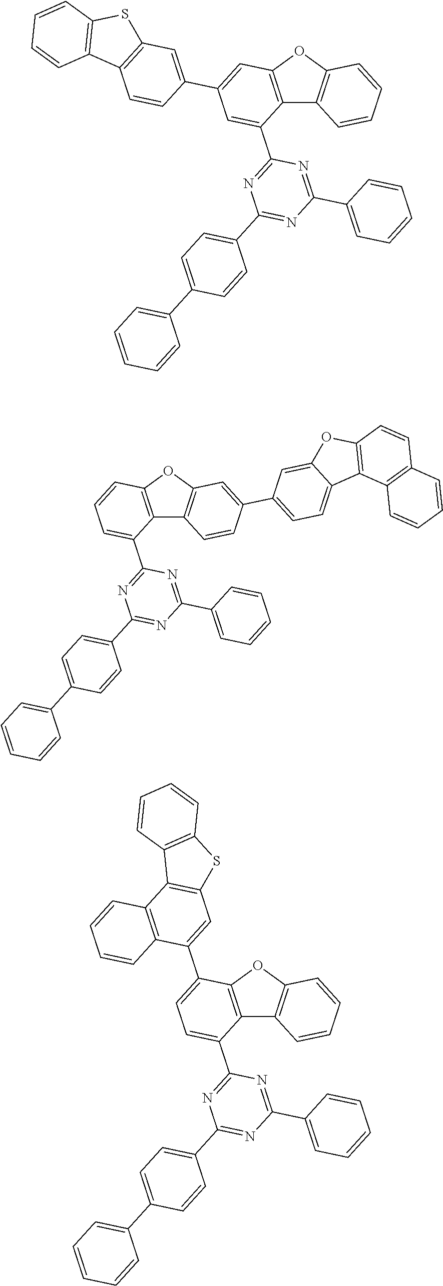 [SMILES: c1ccc(-c2ccc(-c3nc(-c4ccccc4)nc(-c4cc(-c5ccc6c(c5)sc5ccccc56)cc5oc6ccccc6c45)n3)cc2)cc1 ; c1ccc(-c2ccc(-c3nc(-c4ccccc4)nc(-c4cccc5oc6cc(-c7ccc8c(c7)oc7ccc9ccccc9c78)ccc6c45)n3)cc2)cc1 ; c1ccc(-c2ccc(-c3nc(-c4ccccc4)nc(-c4ccc(-c5cc6sc7ccccc7c6c6ccccc56)c5oc6ccccc6c45)n3)cc2)cc1]

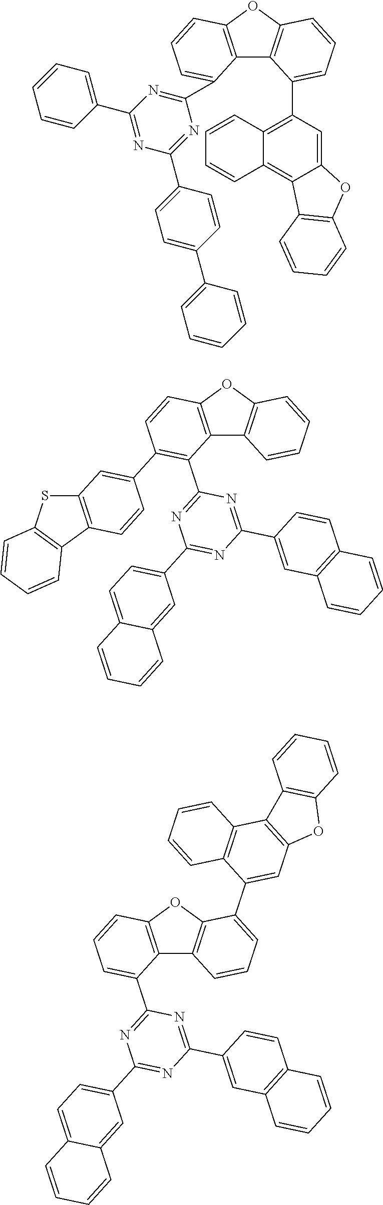

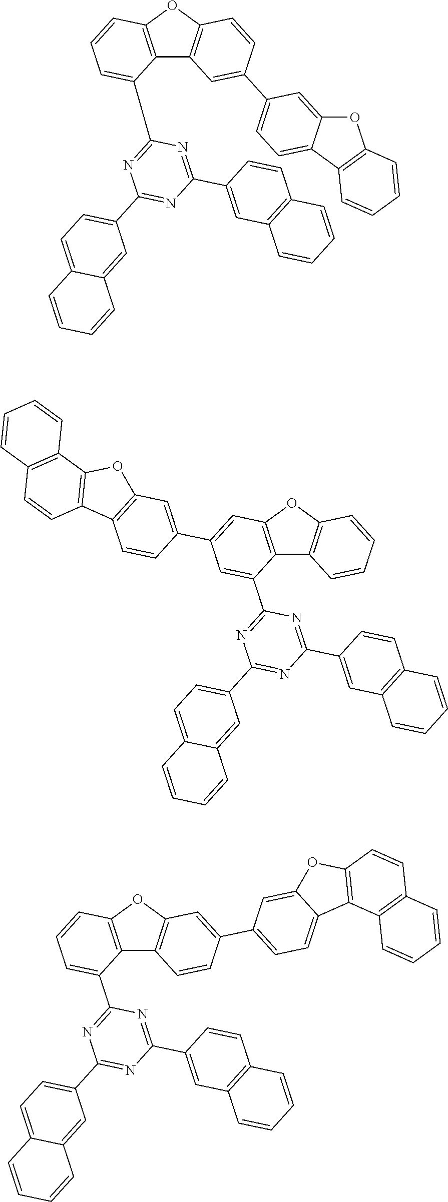 [SMILES: c1ccc2cc(-c3nc(-c4ccc5ccccc5c4)nc(-c4cccc5oc6ccc(-c7ccc8c(c7)oc7ccccc78)cc6c45)n3)ccc2c1 ; c1ccc2cc(-c3nc(-c4ccc5ccccc5c4)nc(-c4cc(-c5ccc6c(c5)oc5c7ccccc7ccc65)cc5oc6ccccc6c45)n3)ccc2c1 ; c1ccc2cc(-c3nc(-c4ccc5ccccc5c4)nc(-c4cccc5oc6cc(-c7ccc8c(c7)oc7ccc9ccccc9c78)ccc6c45)n3)ccc2c1]

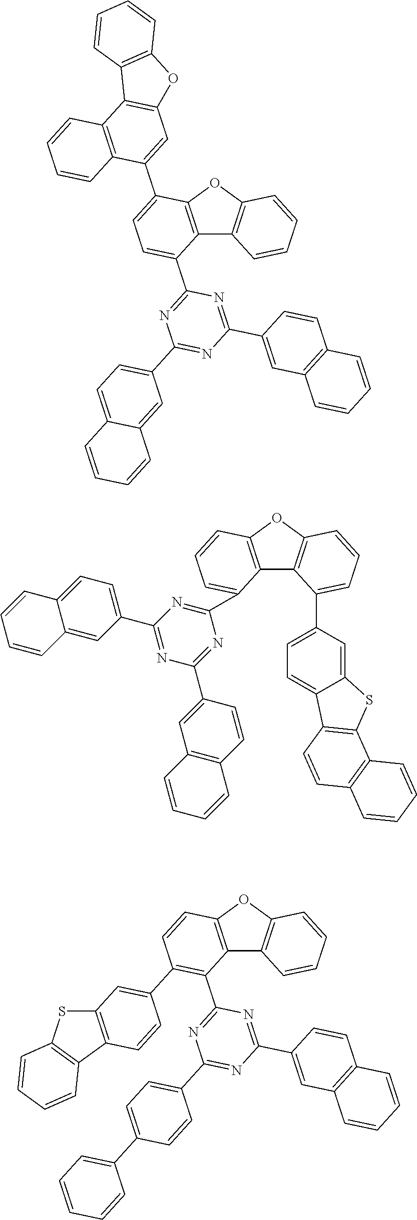 [SMILES: c1ccc2cc(-c3nc(-c4ccc5ccccc5c4)nc(-c4ccc(-c5cc6oc7ccccc7c6c6ccccc56)c5oc6ccccc6c45)n3)ccc2c1 ; c1ccc2cc(-c3nc(-c4ccc5ccccc5c4)nc(-c4cccc5oc6cccc(-c7ccc8c(c7)sc7c9ccccc9ccc87)c6c45)n3)ccc2c1 ; c1ccc(-c2ccc(-c3nc(-c4ccc5ccccc5c4)nc(-c4c(-c5ccc6c(c5)sc5ccccc56)ccc5oc6ccccc6c45)n3)cc2)cc1]

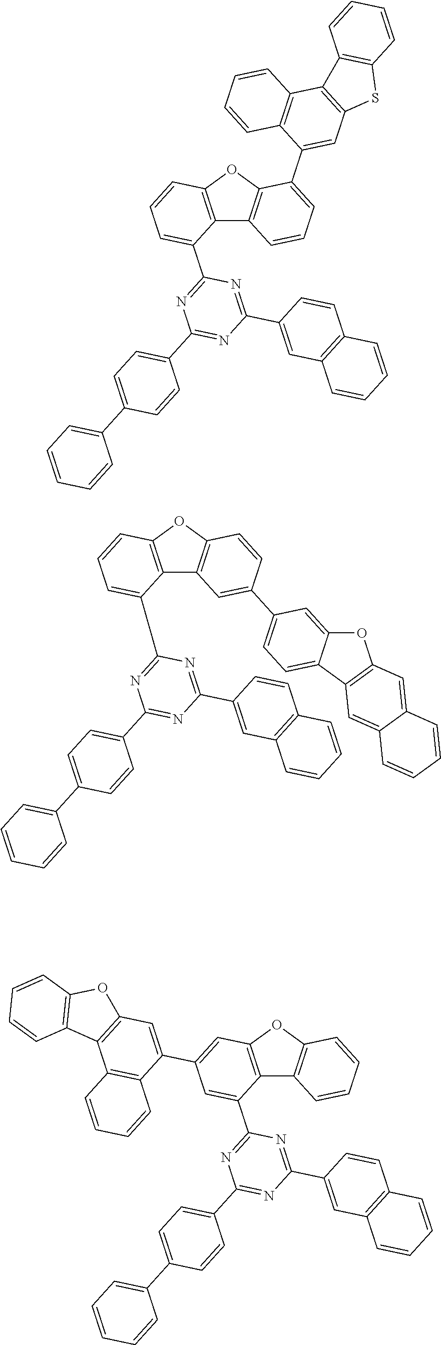 [SMILES: c1ccc(-c2ccc(-c3nc(-c4ccc5ccccc5c4)nc(-c4cccc5oc6c(-c7cc8sc9ccccc9c8c8ccccc78)cccc6c45)n3)cc2)cc1 ; c1ccc(-c2ccc(-c3nc(-c4ccc5ccccc5c4)nc(-c4cccc5oc6ccc(-c7ccc8c(c7)oc7cc9ccccc9cc78)cc6c45)n3)cc2)cc1 ; c1ccc(-c2ccc(-c3nc(-c4ccc5ccccc5c4)nc(-c4cc(-c5cc6oc7ccccc7c6c6ccccc56)cc5oc6ccccc6c45)n3)cc2)cc1]

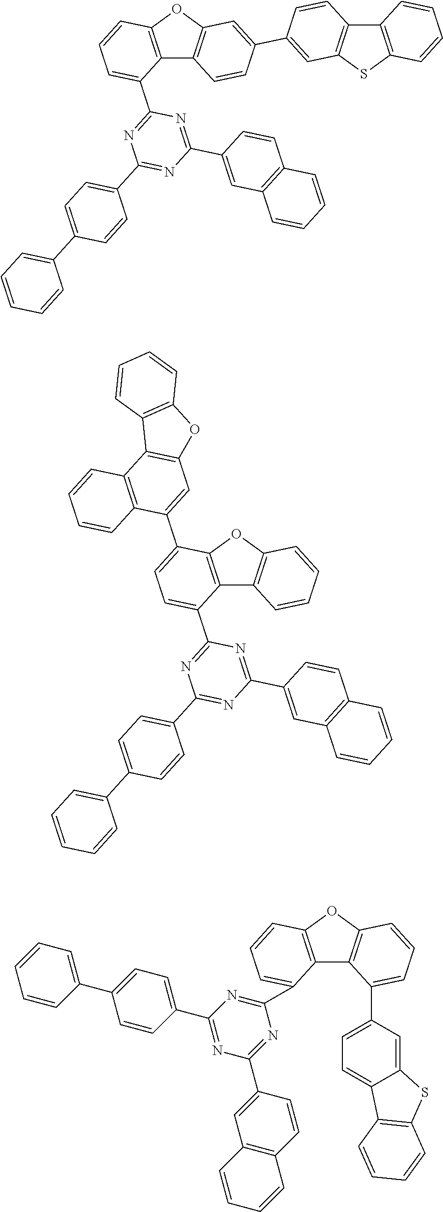 [SMILES: c1ccc(-c2ccc(-c3nc(-c4ccc5ccccc5c4)nc(-c4cccc5oc6cc(-c7ccc8c(c7)sc7ccccc78)ccc6c45)n3)cc2)cc1 ; c1ccc(-c2ccc(-c3nc(-c4ccc5ccccc5c4)nc(-c4ccc(-c5cc6oc7ccccc7c6c6ccccc56)c5oc6ccccc6c45)n3)cc2)cc1 ; c1ccc(-c2ccc(-c3nc(-c4ccc5ccccc5c4)nc(-c4cccc5oc6cccc(-c7ccc8c(c7)sc7ccccc78)c6c45)n3)cc2)cc1]

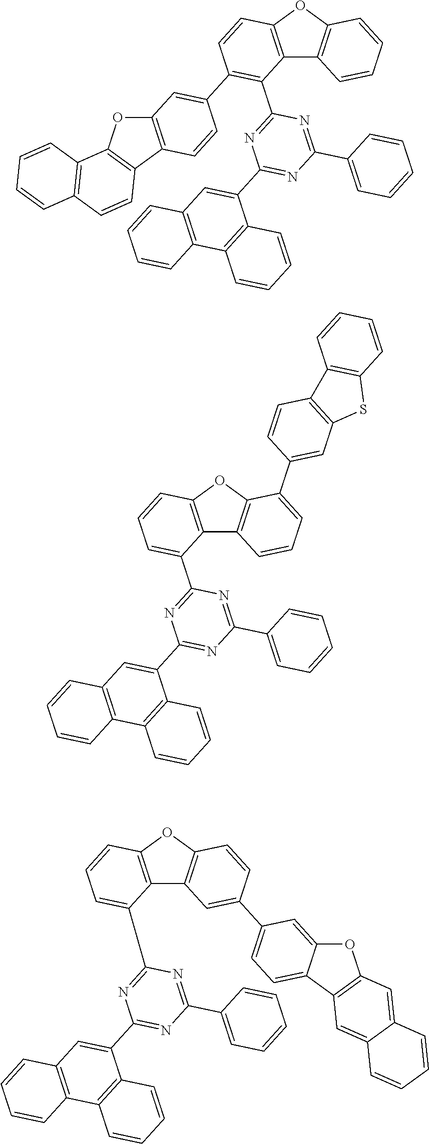 [SMILES: c1ccc(-c2nc(-c3cc4ccccc4c4ccccc34)nc(-c3c(-c4ccc5c(c4)oc4c6ccccc6ccc54)ccc4oc5ccccc5c34)n2)cc1 ; c1ccc(-c2nc(-c3cc4ccccc4c4ccccc34)nc(-c3cccc4oc5c(-c6ccc7c(c6)sc6ccccc67)cccc5c34)n2)cc1 ; c1ccc(-c2nc(-c3cc4ccccc4c4ccccc34)nc(-c3cccc4oc5ccc(-c6ccc7c(c6)oc6cc8ccccc8cc67)cc5c34)n2)cc1]

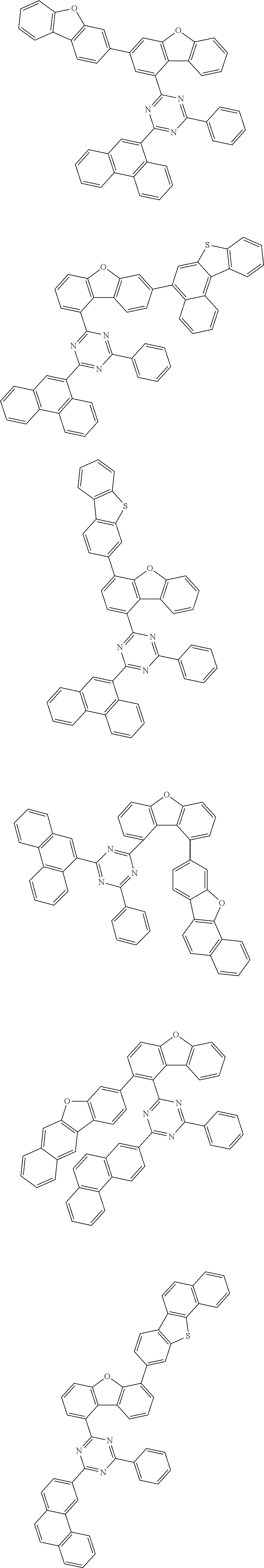

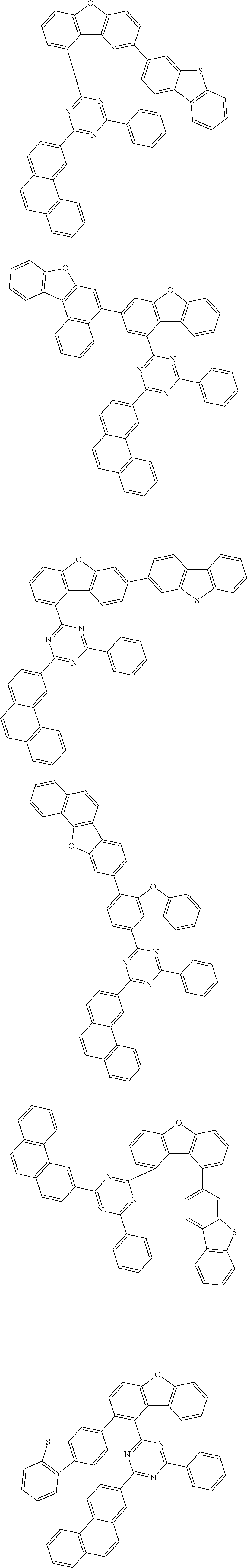

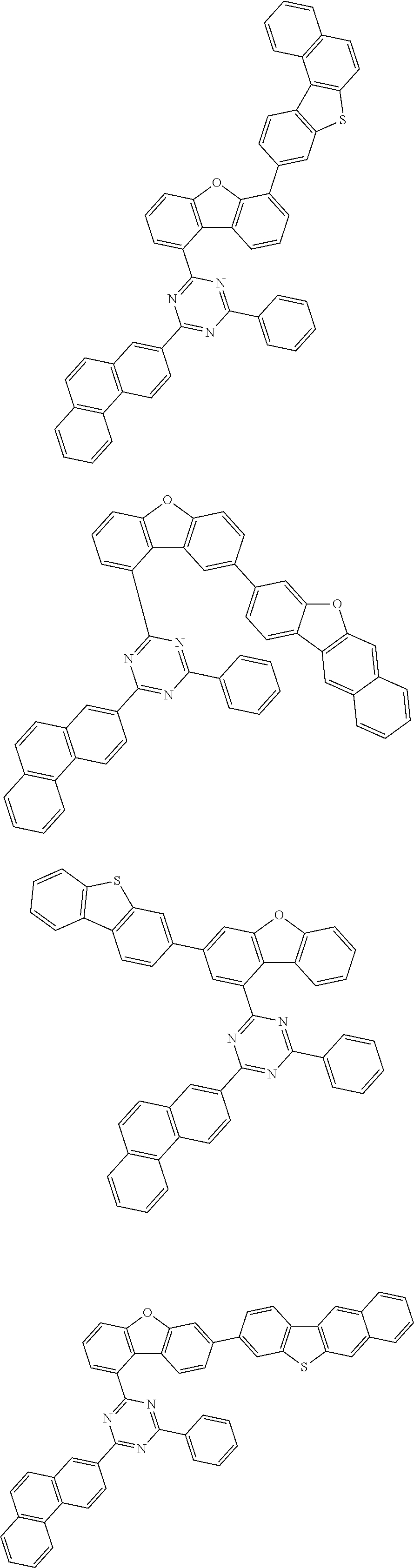

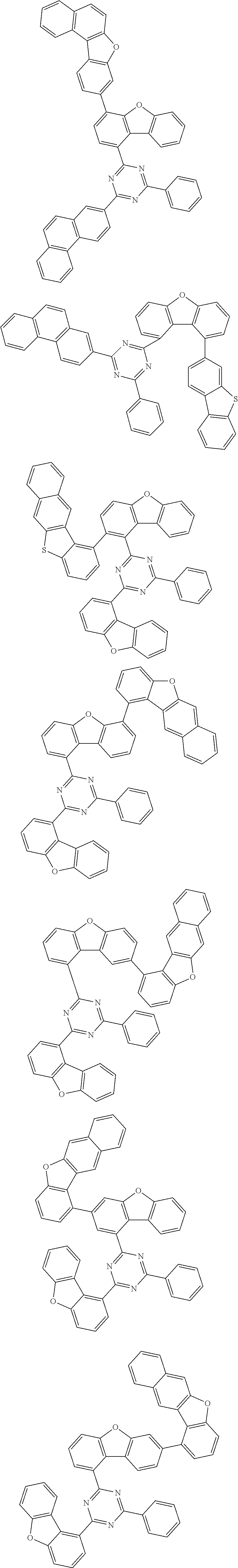

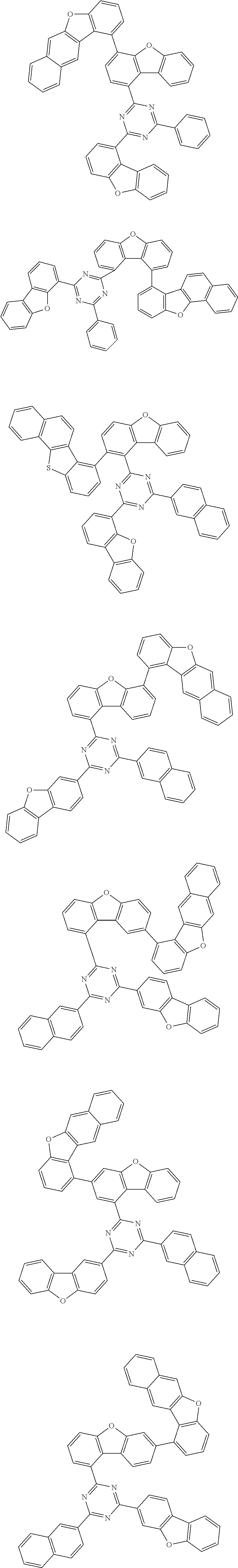

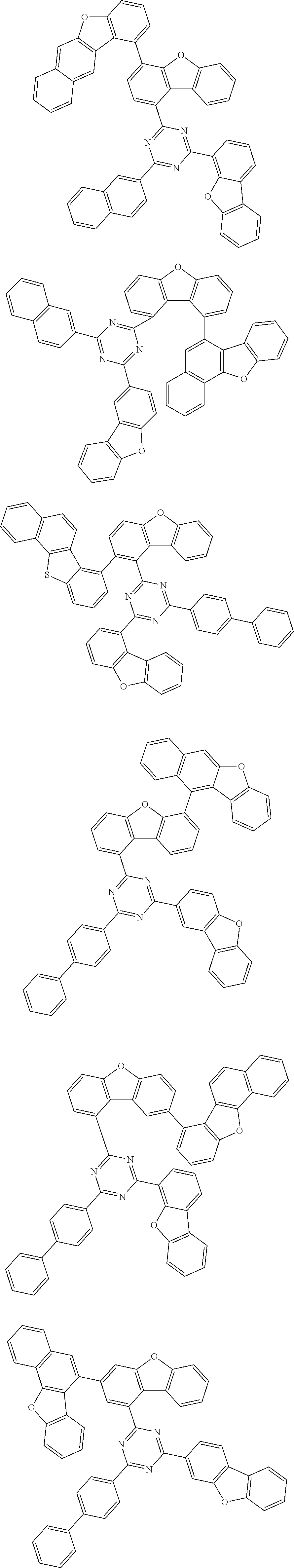

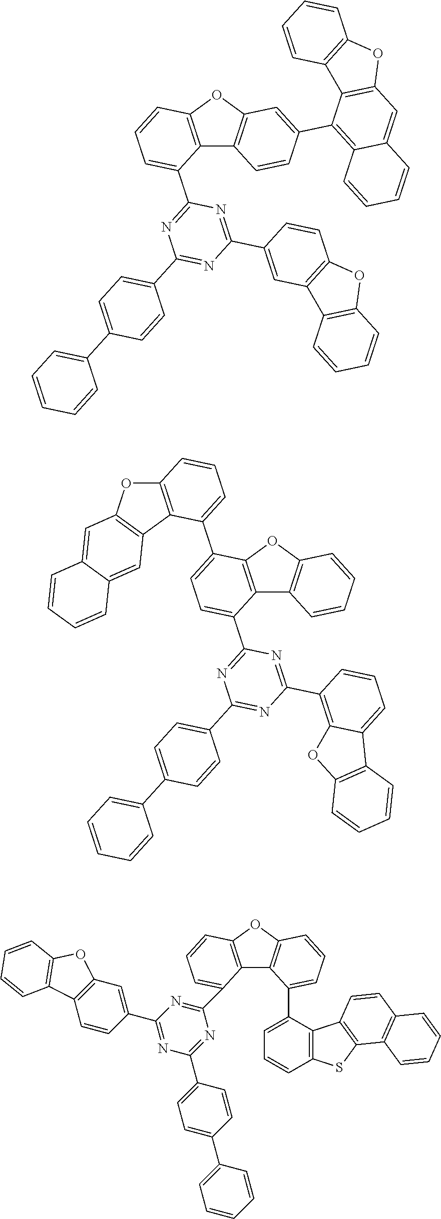 [SMILES: c1ccc(-c2ccc(-c3nc(-c4ccc5oc6ccccc6c5c4)nc(-c4cccc5oc6cc(-c7c8ccccc8cc8oc9ccccc9c78)ccc6c45)n3)cc2)cc1 ; c1ccc(-c2ccc(-c3nc(-c4cccc5c4oc4ccccc45)nc(-c4ccc(-c5cccc6oc7cc8ccccc8cc7c56)c5oc6ccccc6c45)n3)cc2)cc1 ; c1ccc(-c2ccc(-c3nc(-c4ccc5c(c4)oc4ccccc45)nc(-c4cccc5oc6cccc(-c7cccc8sc9c%10ccccc%10ccc9c78)c6c45)n3)cc2)cc1]

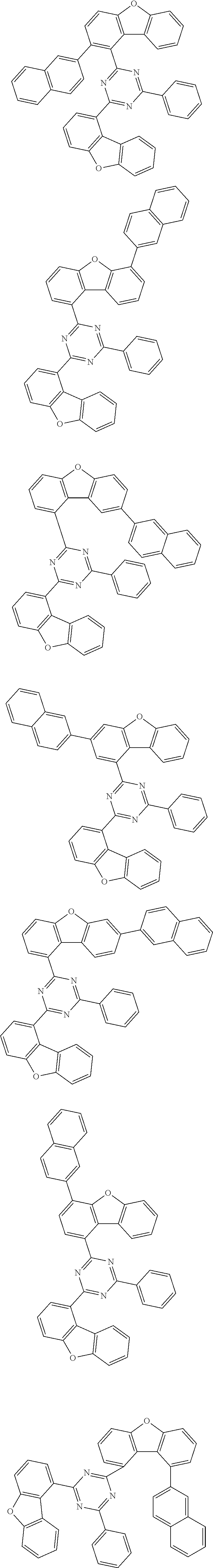

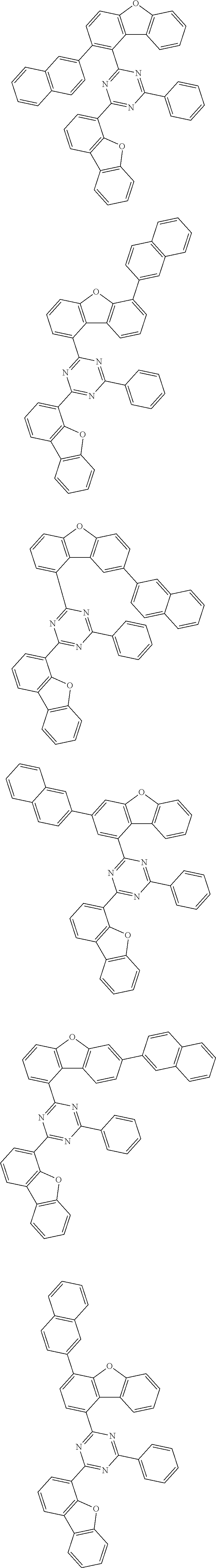 [SMILES: c1ccc(-c2nc(-c3cccc4c3oc3ccccc34)nc(-c3c(-c4ccc5ccccc5c4)ccc4oc5ccccc5c34)n2)cc1 ; c1ccc(-c2nc(-c3cccc4c3oc3ccccc34)nc(-c3cccc4oc5c(-c6ccc7ccccc7c6)cccc5c34)n2)cc1 ; c1ccc(-c2nc(-c3cccc4c3oc3ccccc34)nc(-c3cccc4oc5ccc(-c6ccc7ccccc7c6)cc5c34)n2)cc1 ; c1ccc(-c2nc(-c3cccc4c3oc3ccccc34)nc(-c3cc(-c4ccc5ccccc5c4)cc4oc5ccccc5c34)n2)cc1 ; c1ccc(-c2nc(-c3cccc4c3oc3ccccc34)nc(-c3cccc4oc5cc(-c6ccc7ccccc7c6)ccc5c34)n2)cc1 ; c1ccc(-c2nc(-c3cccc4c3oc3ccccc34)nc(-c3ccc(-c4ccc5ccccc5c4)c4oc5ccccc5c34)n2)cc1]

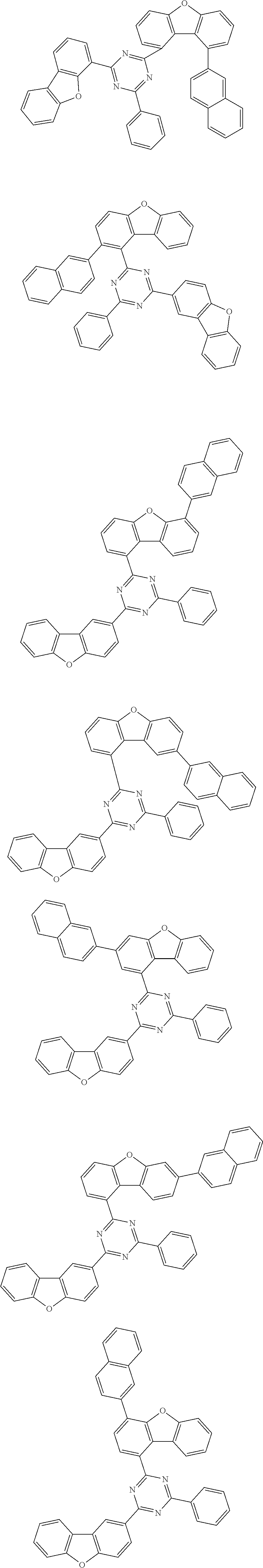

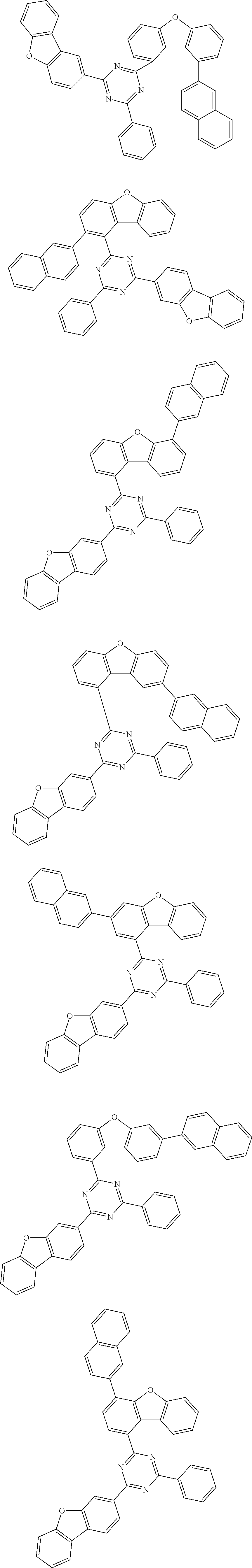

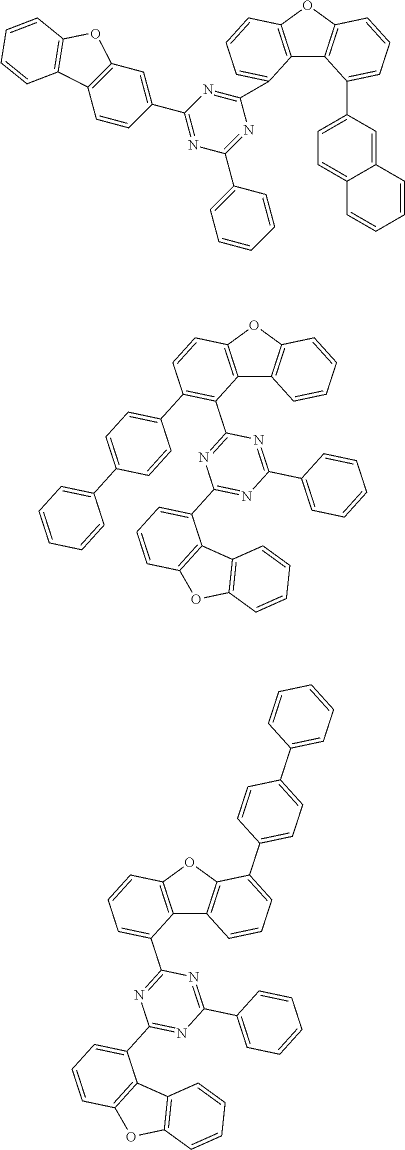 [SMILES: c1ccc(-c2nc(-c3ccc4c(c3)oc3ccccc34)nc(-c3cccc4oc5cccc(-c6ccc7ccccc7c6)c5c34)n2)cc1 ; c1ccc(-c2ccc(-c3ccc4oc5ccccc5c4c3-c3nc(-c4ccccc4)nc(-c4cccc5oc6ccccc6c45)n3)cc2)cc1 ; c1ccc(-c2ccc(-c3cccc4c3oc3cccc(-c5nc(-c6ccccc6)nc(-c6cccc7oc8ccccc8c67)n5)c34)cc2)cc1]

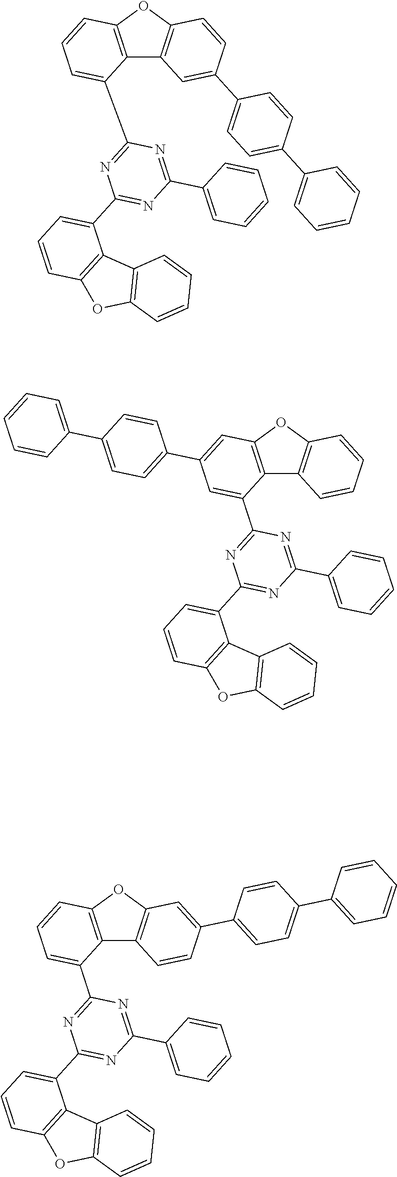 [SMILES: c1ccc(-c2ccc(-c3ccc4oc5cccc(-c6nc(-c7ccccc7)nc(-c7cccc8oc9ccccc9c78)n6)c5c4c3)cc2)cc1 ; c1ccc(-c2ccc(-c3cc(-c4nc(-c5ccccc5)nc(-c5cccc6oc7ccccc7c56)n4)c4c(c3)oc3ccccc34)cc2)cc1 ; c1ccc(-c2ccc(-c3ccc4c(c3)oc3cccc(-c5nc(-c6ccccc6)nc(-c6cccc7oc8ccccc8c67)n5)c34)cc2)cc1]

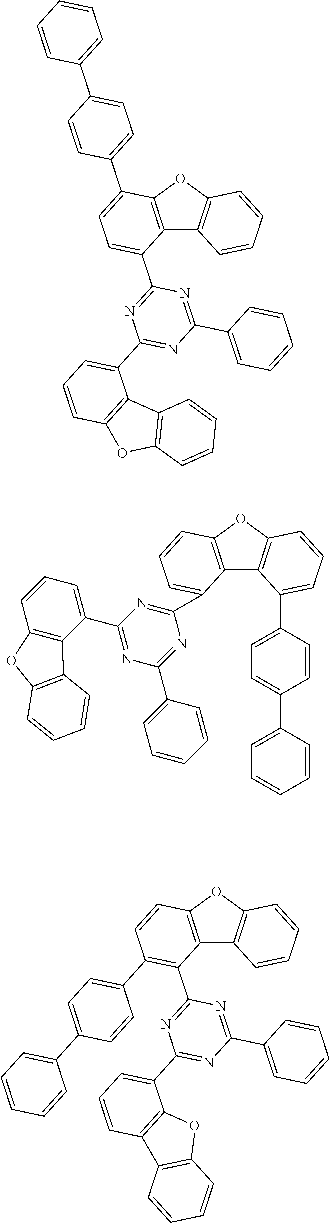 [SMILES: c1ccc(-c2ccc(-c3ccc(-c4nc(-c5ccccc5)nc(-c5cccc6oc7ccccc7c56)n4)c4c3oc3ccccc34)cc2)cc1 ; c1ccc(-c2ccc(-c3cccc4oc5cccc(-c6nc(-c7ccccc7)nc(-c7cccc8oc9ccccc9c78)n6)c5c34)cc2)cc1 ; c1ccc(-c2ccc(-c3ccc4oc5ccccc5c4c3-c3nc(-c4ccccc4)nc(-c4cccc5c4oc4ccccc45)n3)cc2)cc1]

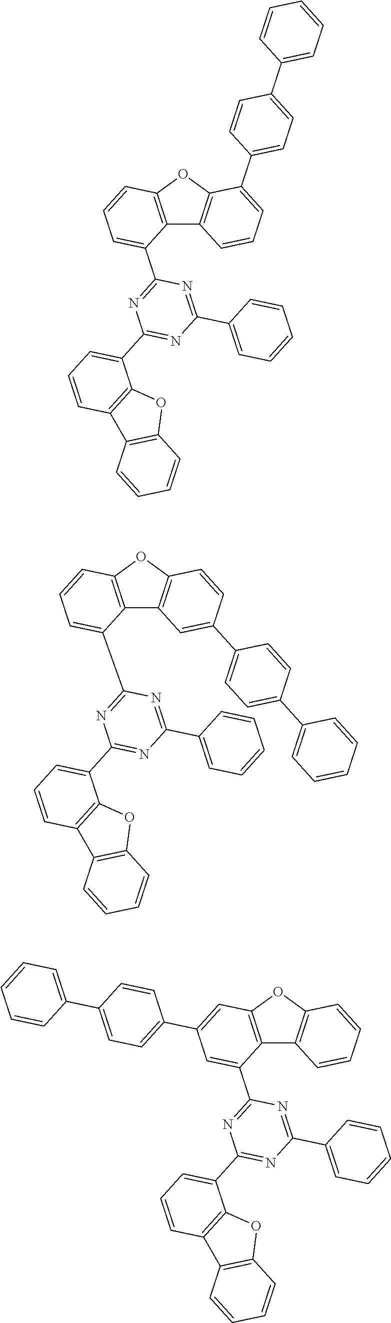 [SMILES: c1ccc(-c2ccc(-c3cccc4c3oc3cccc(-c5nc(-c6ccccc6)nc(-c6cccc7c6oc6ccccc67)n5)c34)cc2)cc1 ; c1ccc(-c2ccc(-c3ccc4oc5cccc(-c6nc(-c7ccccc7)nc(-c7cccc8c7oc7ccccc78)n6)c5c4c3)cc2)cc1 ; c1ccc(-c2ccc(-c3cc(-c4nc(-c5ccccc5)nc(-c5cccc6c5oc5ccccc56)n4)c4c(c3)oc3ccccc34)cc2)cc1]

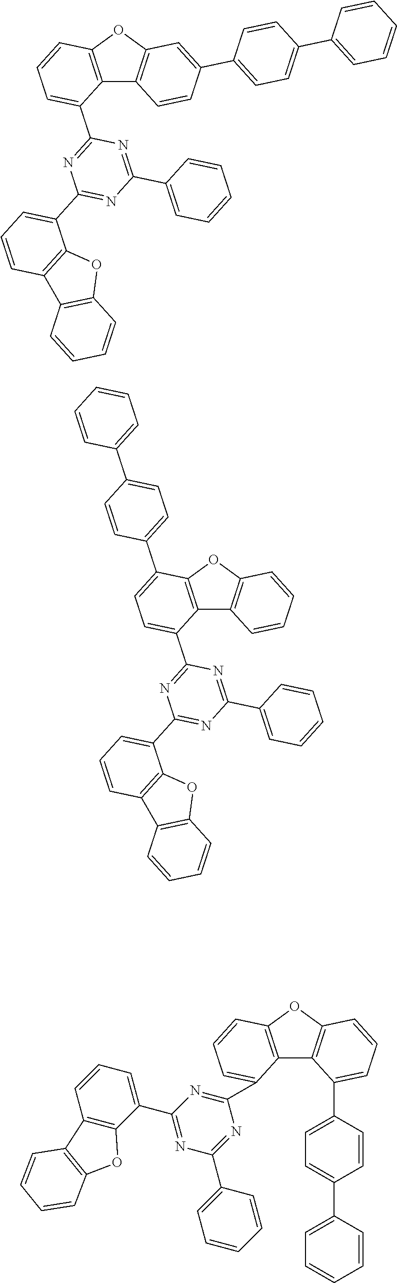

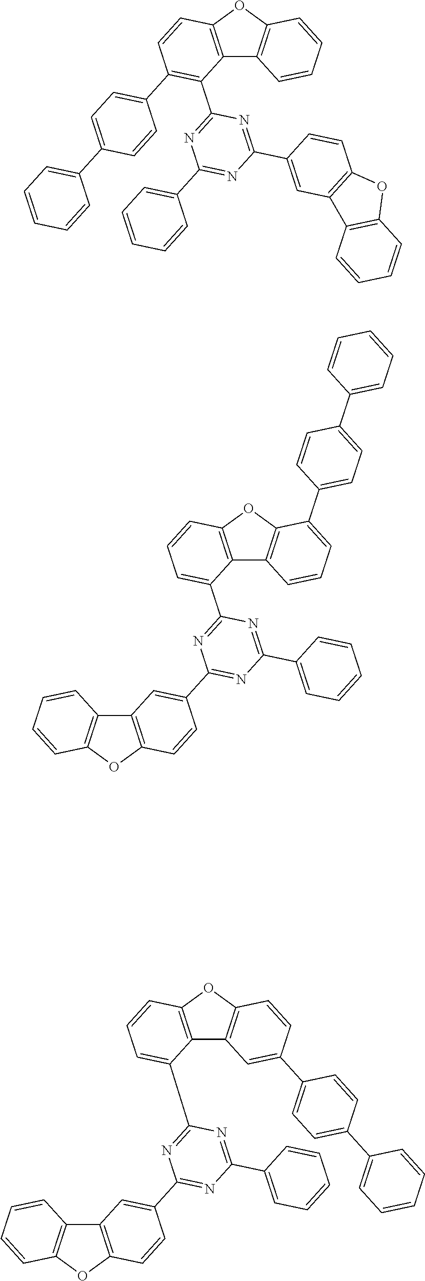 [SMILES: c1ccc(-c2ccc(-c3ccc4oc5ccccc5c4c3-c3nc(-c4ccccc4)nc(-c4ccc5oc6ccccc6c5c4)n3)cc2)cc1 ; c1ccc(-c2ccc(-c3cccc4c3oc3cccc(-c5nc(-c6ccccc6)nc(-c6ccc7oc8ccccc8c7c6)n5)c34)cc2)cc1 ; c1ccc(-c2ccc(-c3ccc4oc5cccc(-c6nc(-c7ccccc7)nc(-c7ccc8oc9ccccc9c8c7)n6)c5c4c3)cc2)cc1]

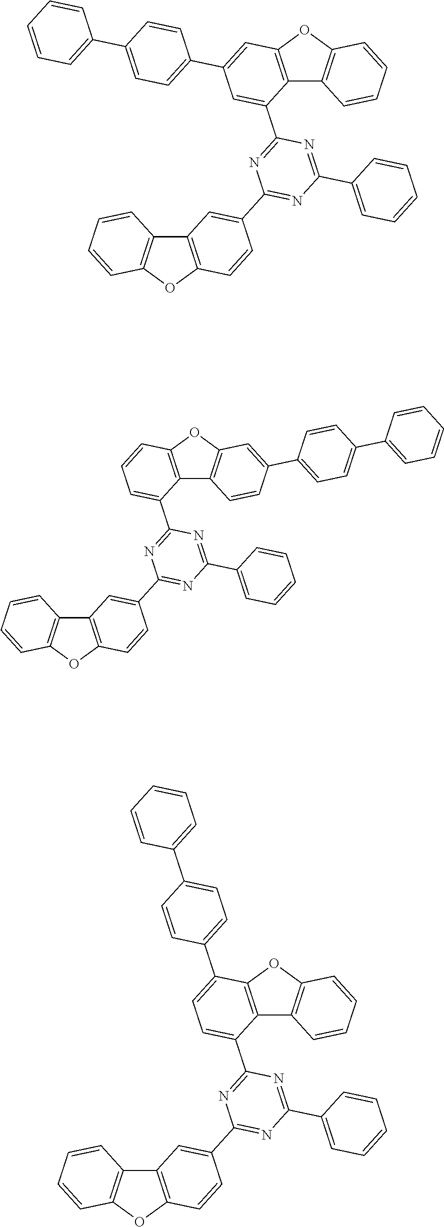 [SMILES: c1ccc(-c2ccc(-c3cc(-c4nc(-c5ccccc5)nc(-c5ccc6oc7ccccc7c6c5)n4)c4c(c3)oc3ccccc34)cc2)cc1 ; c1ccc(-c2ccc(-c3ccc4c(c3)oc3cccc(-c5nc(-c6ccccc6)nc(-c6ccc7oc8ccccc8c7c6)n5)c34)cc2)cc1 ; c1ccc(-c2ccc(-c3ccc(-c4nc(-c5ccccc5)nc(-c5ccc6oc7ccccc7c6c5)n4)c4c3oc3ccccc34)cc2)cc1]

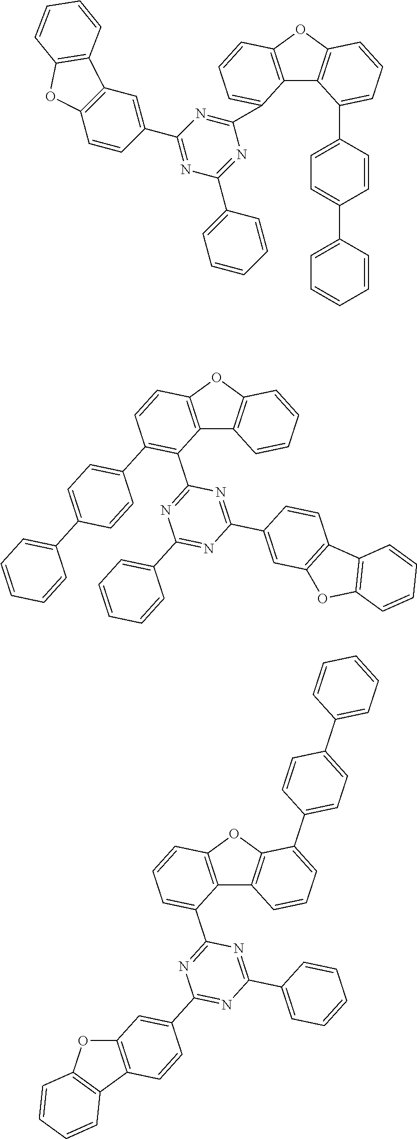 [SMILES: c1ccc(-c2ccc(-c3cccc4oc5cccc(-c6nc(-c7ccccc7)nc(-c7ccc8oc9ccccc9c8c7)n6)c5c34)cc2)cc1 ; c1ccc(-c2ccc(-c3ccc4oc5ccccc5c4c3-c3nc(-c4ccccc4)nc(-c4ccc5c(c4)oc4ccccc45)n3)cc2)cc1 ; c1ccc(-c2ccc(-c3cccc4c3oc3cccc(-c5nc(-c6ccccc6)nc(-c6ccc7c(c6)oc6ccccc67)n5)c34)cc2)cc1]

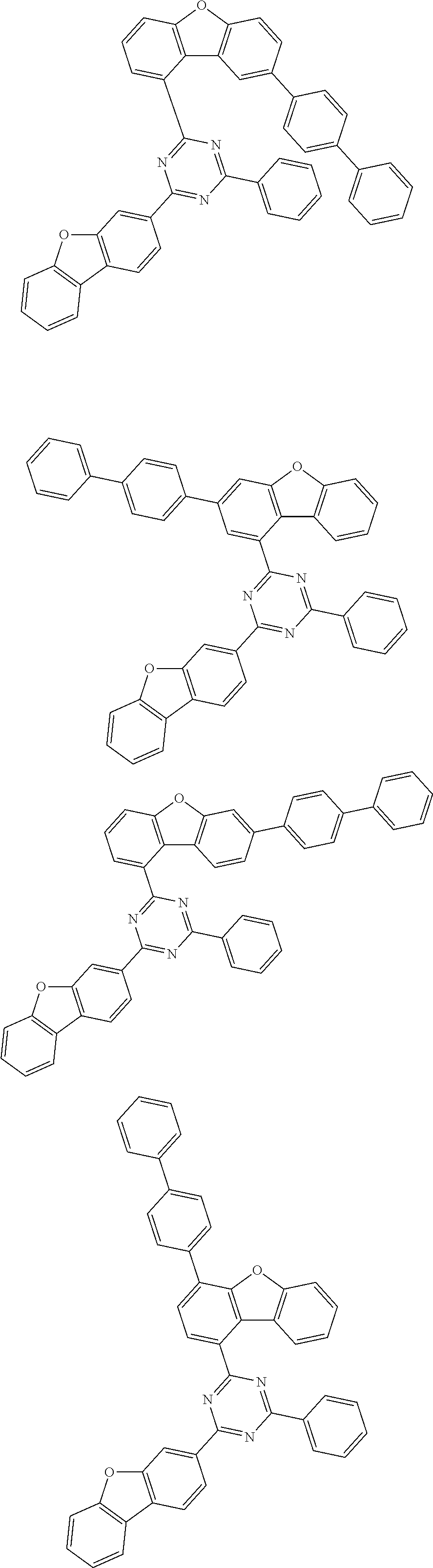 [SMILES: c1ccc(-c2ccc(-c3ccc4oc5cccc(-c6nc(-c7ccccc7)nc(-c7ccc8c(c7)oc7ccccc78)n6)c5c4c3)cc2)cc1 ; c1ccc(-c2ccc(-c3cc(-c4nc(-c5ccccc5)nc(-c5ccc6c(c5)oc5ccccc56)n4)c4c(c3)oc3ccccc34)cc2)cc1 ; c1ccc(-c2ccc(-c3ccc4c(c3)oc3cccc(-c5nc(-c6ccccc6)nc(-c6ccc7c(c6)oc6ccccc67)n5)c34)cc2)cc1 ; c1ccc(-c2ccc(-c3ccc(-c4nc(-c5ccccc5)nc(-c5ccc6c(c5)oc5ccccc56)n4)c4c3oc3ccccc34)cc2)cc1]

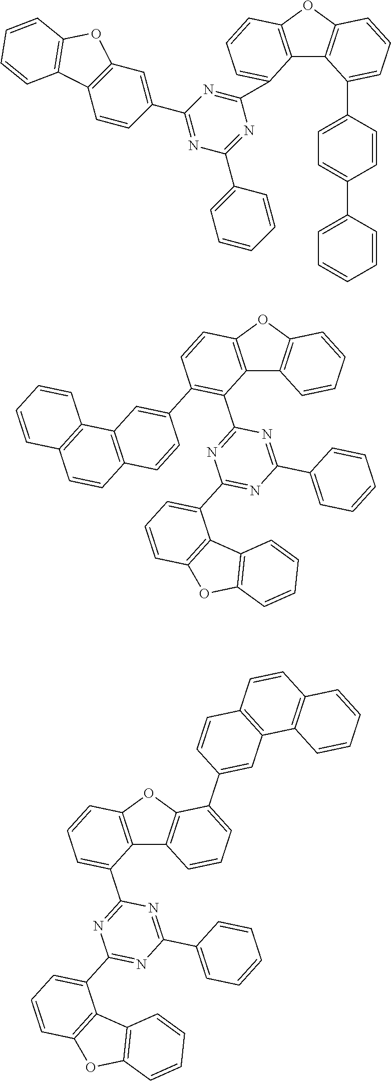 [SMILES: c1ccc(-c2ccc(-c3cccc4oc5cccc(-c6nc(-c7ccccc7)nc(-c7ccc8c(c7)oc7ccccc78)n6)c5c34)cc2)cc1 ; c1ccc(-c2nc(-c3cccc4oc5ccccc5c34)nc(-c3c(-c4ccc5ccc6ccccc6c5c4)ccc4oc5ccccc5c34)n2)cc1 ; c1ccc(-c2nc(-c3cccc4oc5ccccc5c34)nc(-c3cccc4oc5c(-c6ccc7ccc8ccccc8c7c6)cccc5c34)n2)cc1]

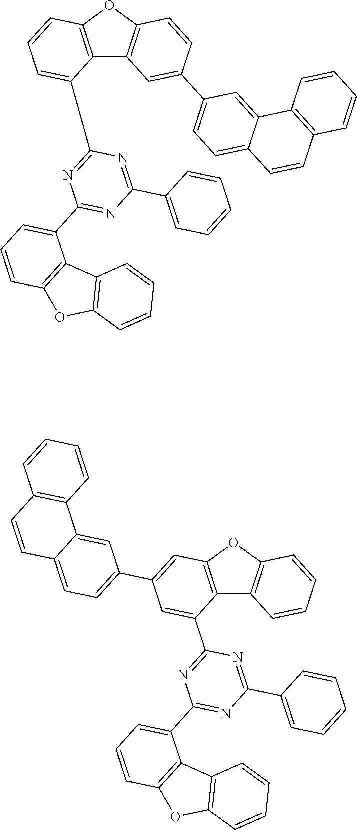 [SMILES: c1ccc(-c2nc(-c3cccc4oc5ccccc5c34)nc(-c3cccc4oc5ccc(-c6ccc7ccc8ccccc8c7c6)cc5c34)n2)cc1 ; c1ccc(-c2nc(-c3cccc4oc5ccccc5c34)nc(-c3cc(-c4ccc5ccc6ccccc6c5c4)cc4oc5ccccc5c34)n2)cc1]

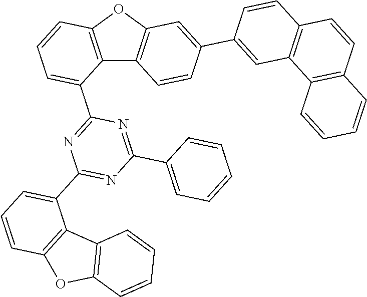 [SMILES: c1ccc(-c2nc(-c3cccc4oc5ccccc5c34)nc(-c3cccc4oc5cc(-c6ccc7ccc8ccccc8c7c6)ccc5c34)n2)cc1]

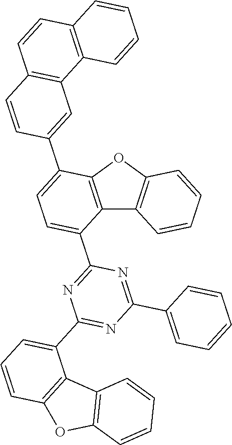 [SMILES: c1ccc(-c2nc(-c3cccc4oc5ccccc5c34)nc(-c3ccc(-c4ccc5ccc6ccccc6c5c4)c4oc5ccccc5c34)n2)cc1]

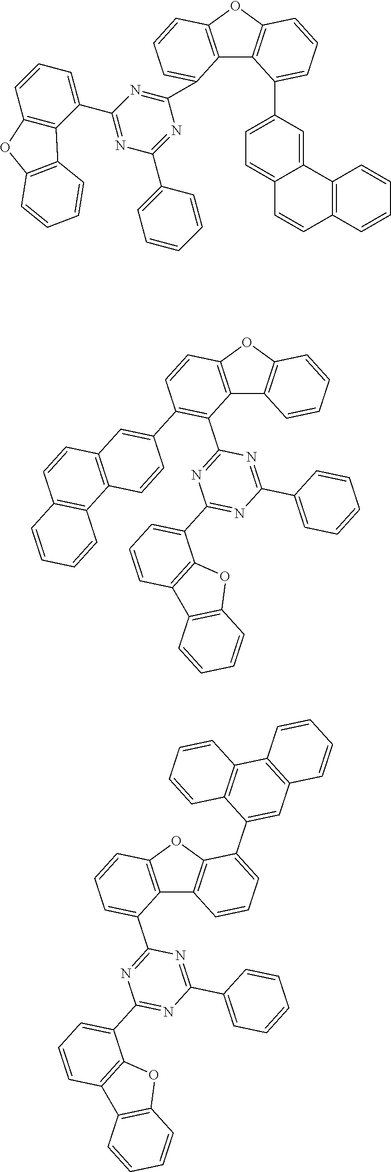 [SMILES: c1ccc(-c2nc(-c3cccc4oc5ccccc5c34)nc(-c3cccc4oc5cccc(-c6ccc7ccc8ccccc8c7c6)c5c34)n2)cc1 ; c1ccc(-c2nc(-c3cccc4c3oc3ccccc34)nc(-c3c(-c4ccc5c(ccc6ccccc65)c4)ccc4oc5ccccc5c34)n2)cc1 ; c1ccc(-c2nc(-c3cccc4c3oc3ccccc34)nc(-c3cccc4oc5c(-c6cc7ccccc7c7ccccc67)cccc5c34)n2)cc1]

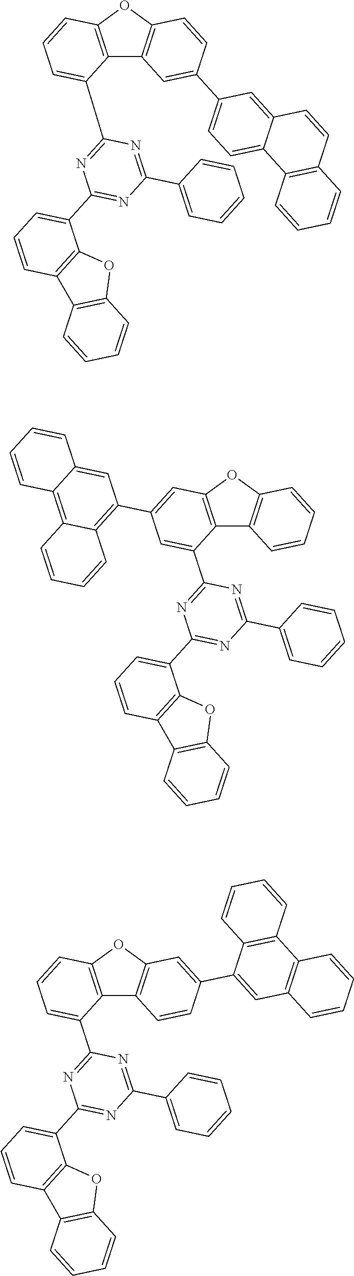 [SMILES: c1ccc(-c2nc(-c3cccc4c3oc3ccccc34)nc(-c3cccc4oc5ccc(-c6ccc7c(ccc8ccccc87)c6)cc5c34)n2)cc1 ; c1ccc(-c2nc(-c3cccc4c3oc3ccccc34)nc(-c3cc(-c4cc5ccccc5c5ccccc45)cc4oc5ccccc5c34)n2)cc1 ; c1ccc(-c2nc(-c3cccc4c3oc3ccccc34)nc(-c3cccc4oc5cc(-c6cc7ccccc7c7ccccc67)ccc5c34)n2)cc1]

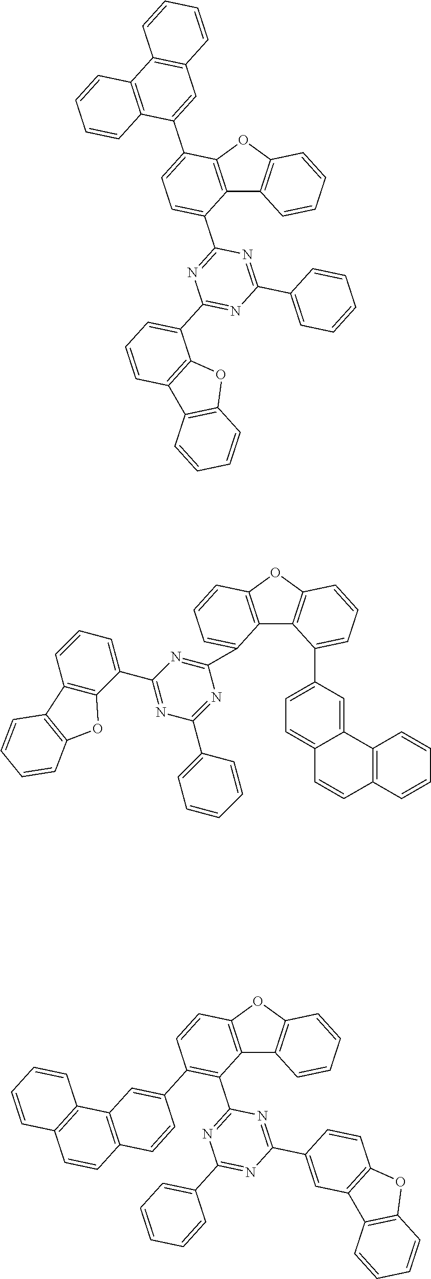 [SMILES: c1ccc(-c2nc(-c3cccc4c3oc3ccccc34)nc(-c3ccc(-c4cc5ccccc5c5ccccc45)c4oc5ccccc5c34)n2)cc1 ; c1ccc(-c2nc(-c3cccc4c3oc3ccccc34)nc(-c3cccc4oc5cccc(-c6ccc7ccc8ccccc8c7c6)c5c34)n2)cc1 ; c1ccc(-c2nc(-c3ccc4oc5ccccc5c4c3)nc(-c3c(-c4ccc5ccc6ccccc6c5c4)ccc4oc5ccccc5c34)n2)cc1]

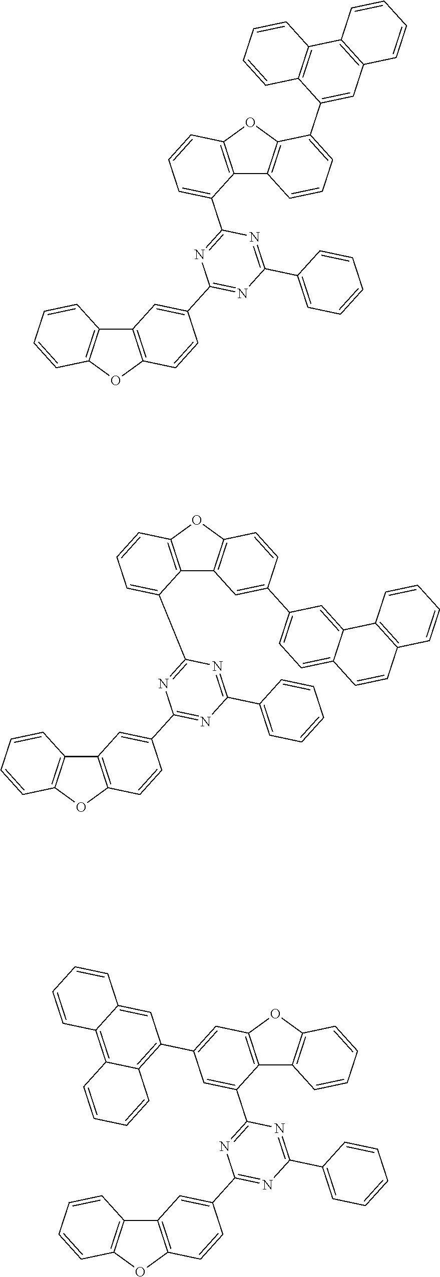 [SMILES: c1ccc(-c2nc(-c3ccc4oc5ccccc5c4c3)nc(-c3cccc4oc5c(-c6cc7ccccc7c7ccccc67)cccc5c34)n2)cc1 ; c1ccc(-c2nc(-c3ccc4oc5ccccc5c4c3)nc(-c3cccc4oc5ccc(-c6ccc7ccc8ccccc8c7c6)cc5c34)n2)cc1 ; c1ccc(-c2nc(-c3ccc4oc5ccccc5c4c3)nc(-c3cc(-c4cc5ccccc5c5ccccc45)cc4oc5ccccc5c34)n2)cc1]

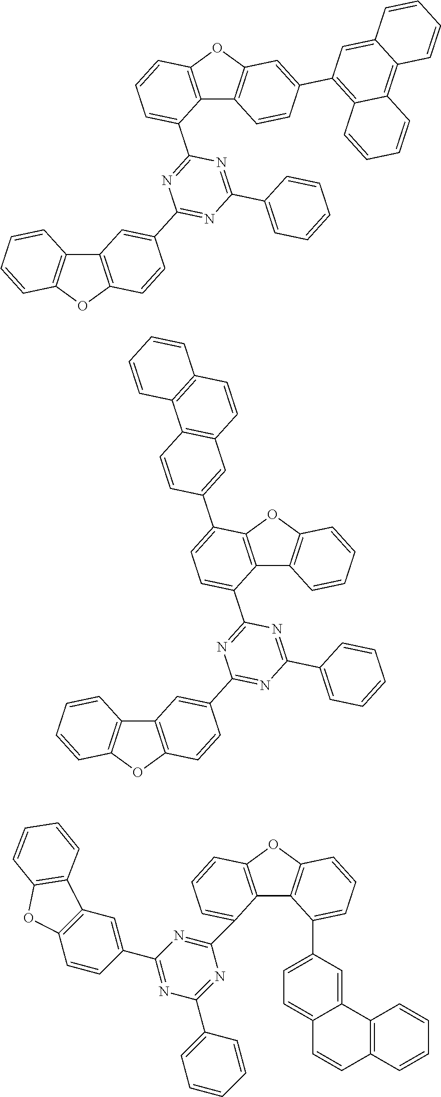

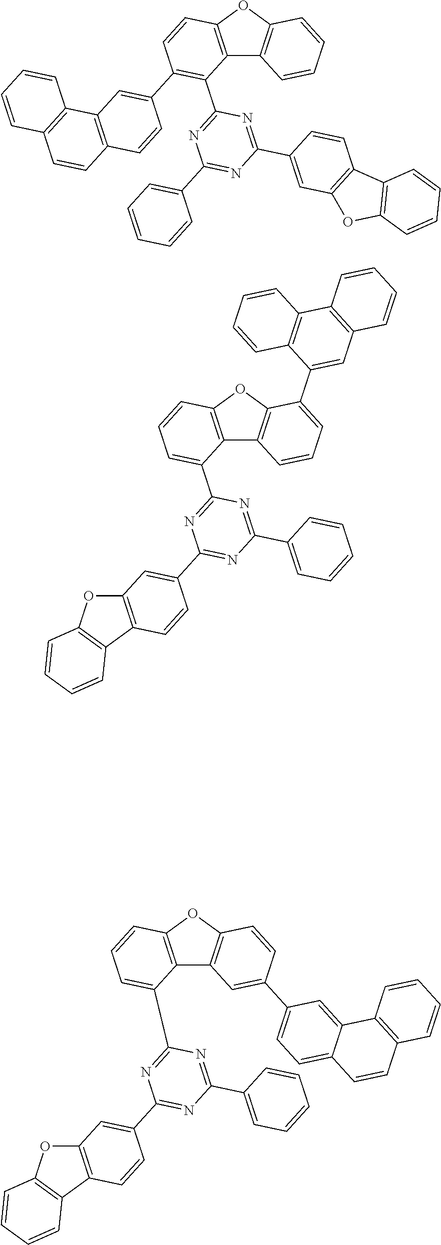 [SMILES: c1ccc(-c2nc(-c3ccc4c(c3)oc3ccccc34)nc(-c3c(-c4ccc5ccc6ccccc6c5c4)ccc4oc5ccccc5c34)n2)cc1 ; c1ccc(-c2nc(-c3ccc4c(c3)oc3ccccc34)nc(-c3cccc4oc5c(-c6cc7ccccc7c7ccccc67)cccc5c34)n2)cc1 ; c1ccc(-c2nc(-c3ccc4c(c3)oc3ccccc34)nc(-c3cccc4oc5ccc(-c6ccc7ccc8ccccc8c7c6)cc5c34)n2)cc1]

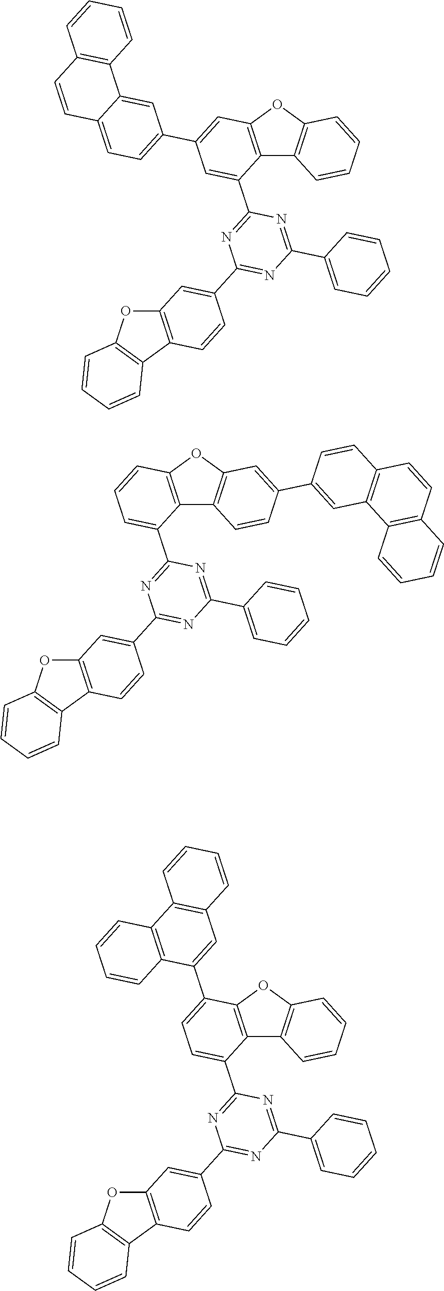 [SMILES: c1ccc(-c2nc(-c3ccc4c(c3)oc3ccccc34)nc(-c3cc(-c4ccc5ccc6ccccc6c5c4)cc4oc5ccccc5c34)n2)cc1 ; c1ccc(-c2nc(-c3ccc4c(c3)oc3ccccc34)nc(-c3cccc4oc5cc(-c6ccc7ccc8ccccc8c7c6)ccc5c34)n2)cc1 ; c1ccc(-c2nc(-c3ccc4c(c3)oc3ccccc34)nc(-c3ccc(-c4cc5ccccc5c5ccccc45)c4oc5ccccc5c34)n2)cc1]

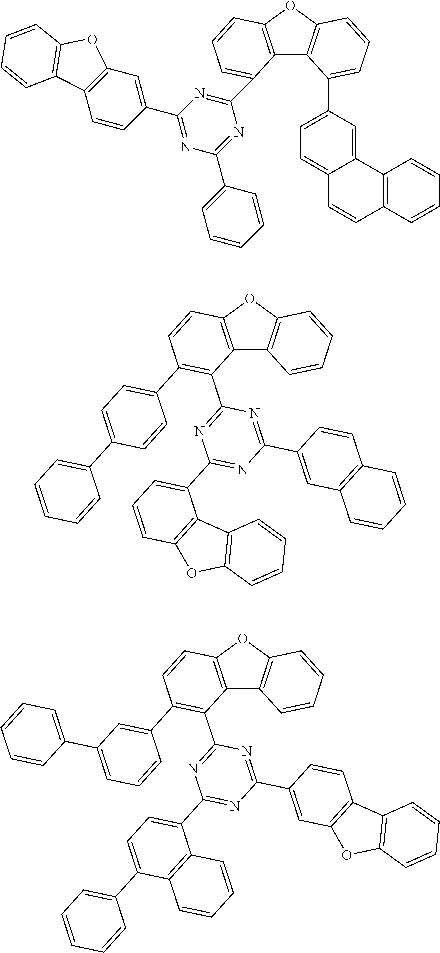 [SMILES: c1ccc(-c2nc(-c3ccc4c(c3)oc3ccccc34)nc(-c3cccc4oc5cccc(-c6ccc7ccc8ccccc8c7c6)c5c34)n2)cc1 ; c1ccc(-c2ccc(-c3ccc4oc5ccccc5c4c3-c3nc(-c4ccc5ccccc5c4)nc(-c4cccc5oc6ccccc6c45)n3)cc2)cc1 ; c1ccc(-c2cccc(-c3ccc4oc5ccccc5c4c3-c3nc(-c4ccc5c(c4)oc4ccccc45)nc(-c4ccc(-c5ccccc5)c5ccccc45)n3)c2)cc1]

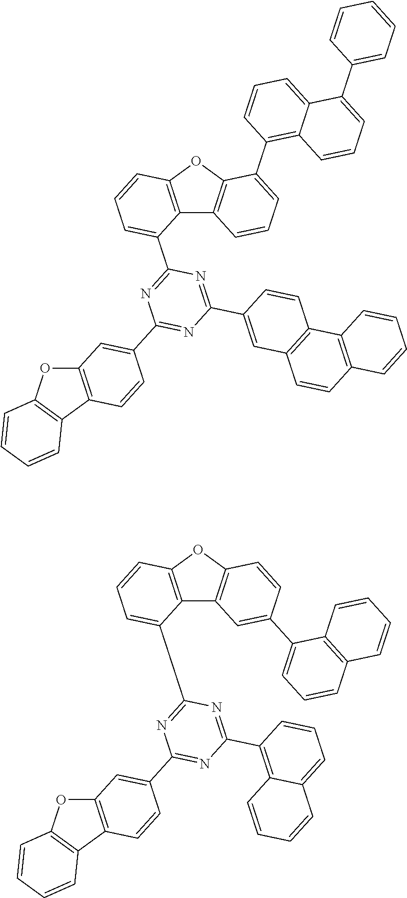 [SMILES: c1ccc(-c2cccc3c(-c4cccc5c4oc4cccc(-c6nc(-c7ccc8c(ccc9ccccc98)c7)nc(-c7ccc8c(c7)oc7ccccc78)n6)c45)cccc23)cc1 ; c1ccc2c(-c3ccc4oc5cccc(-c6nc(-c7ccc8c(c7)oc7ccccc78)nc(-c7cccc8ccccc78)n6)c5c4c3)cccc2c1]

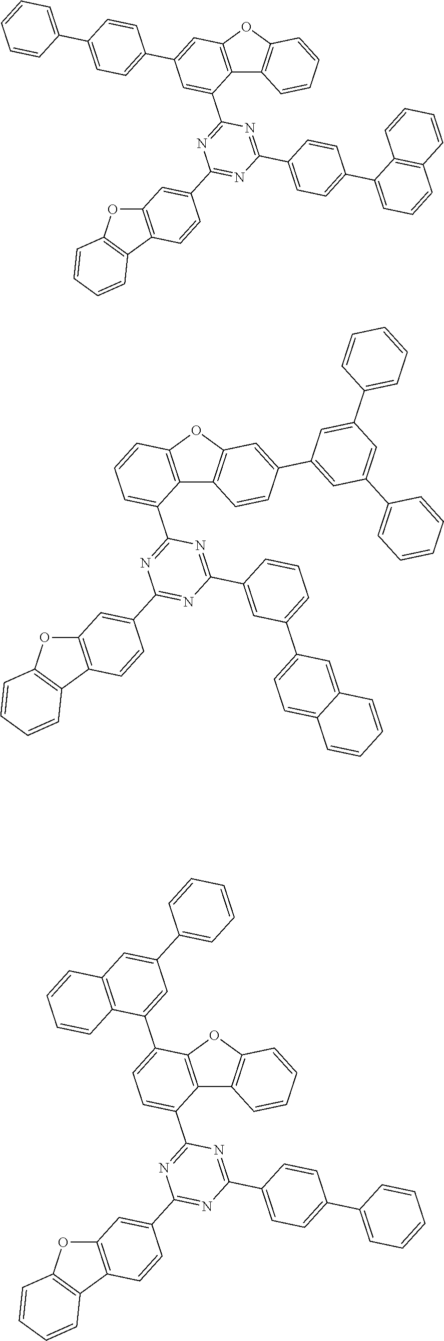 [SMILES: c1ccc(-c2ccc(-c3cc(-c4nc(-c5ccc(-c6cccc7ccccc67)cc5)nc(-c5ccc6c(c5)oc5ccccc56)n4)c4c(c3)oc3ccccc34)cc2)cc1 ; c1ccc(-c2cc(-c3ccccc3)cc(-c3ccc4c(c3)oc3cccc(-c5nc(-c6cccc(-c7ccc8ccccc8c7)c6)nc(-c6ccc7c(c6)oc6ccccc67)n5)c34)c2)cc1 ; c1ccc(-c2ccc(-c3nc(-c4ccc5c(c4)oc4ccccc45)nc(-c4ccc(-c5cc(-c6ccccc6)cc6ccccc56)c5oc6ccccc6c45)n3)cc2)cc1]

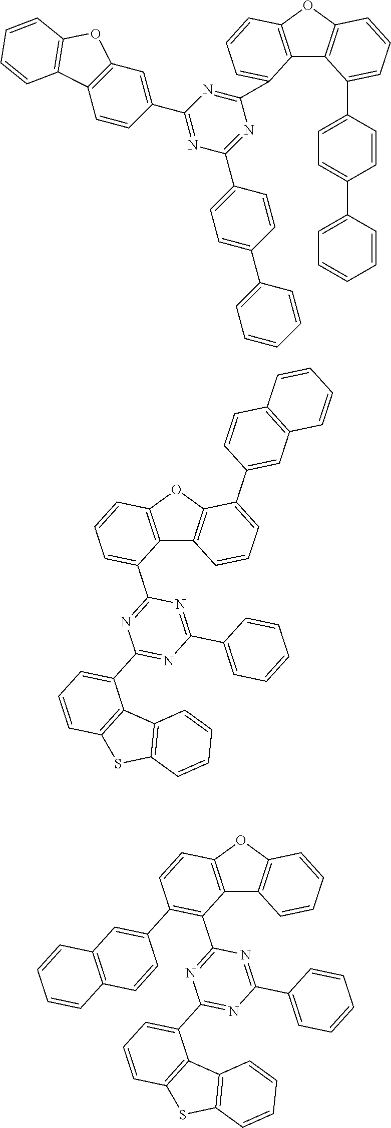

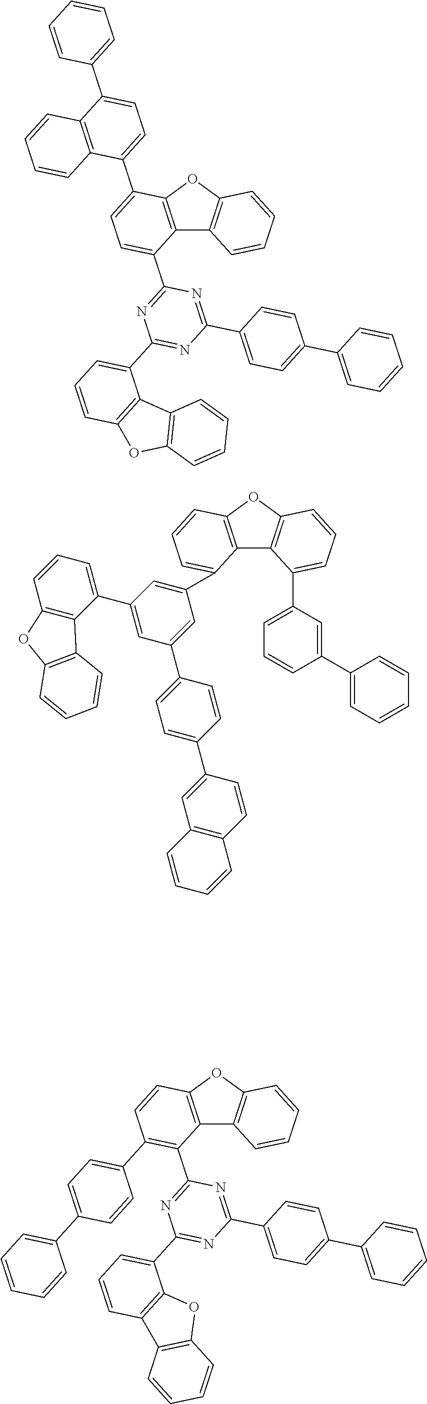

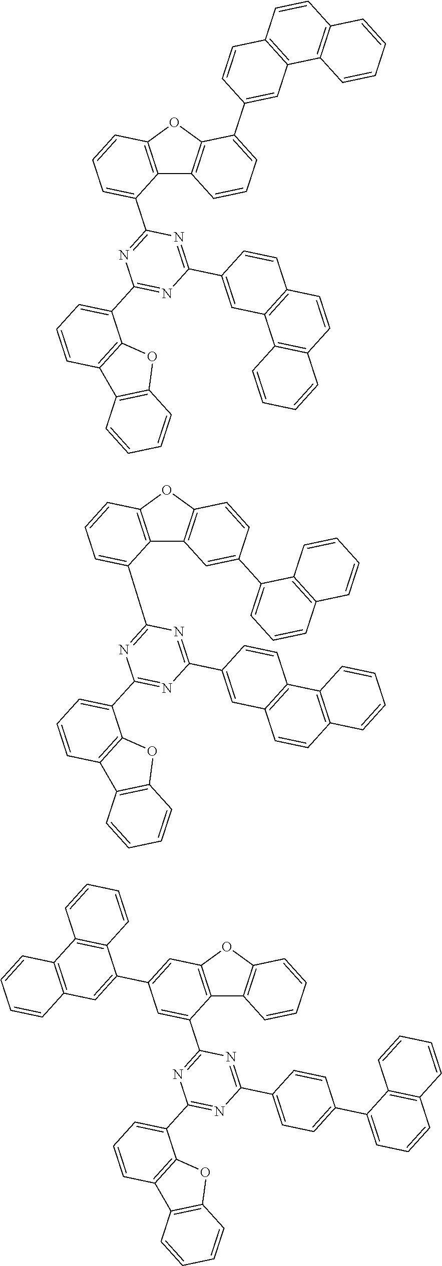

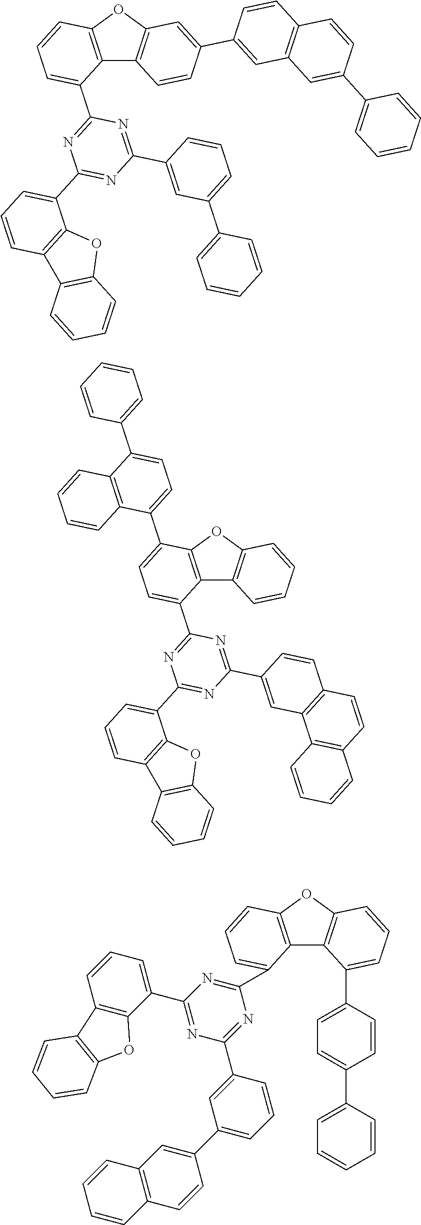

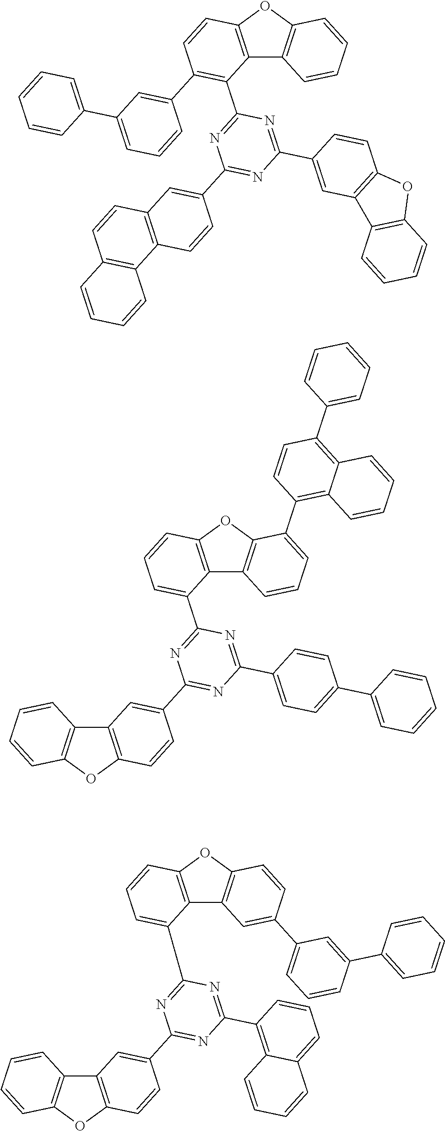 [SMILES: c1ccc(-c2cccc(-c3ccc4oc5ccccc5c4c3-c3nc(-c4ccc5c(ccc6ccccc65)c4)nc(-c4ccc5oc6ccccc6c5c4)n3)c2)cc1 ; c1ccc(-c2ccc(-c3nc(-c4ccc5oc6ccccc6c5c4)nc(-c4cccc5oc6c(-c7ccc(-c8ccccc8)c8ccccc78)cccc6c45)n3)cc2)cc1 ; c1ccc(-c2cccc(-c3ccc4oc5cccc(-c6nc(-c7ccc8oc9ccccc9c8c7)nc(-c7cccc8ccccc78)n6)c5c4c3)c2)cc1]

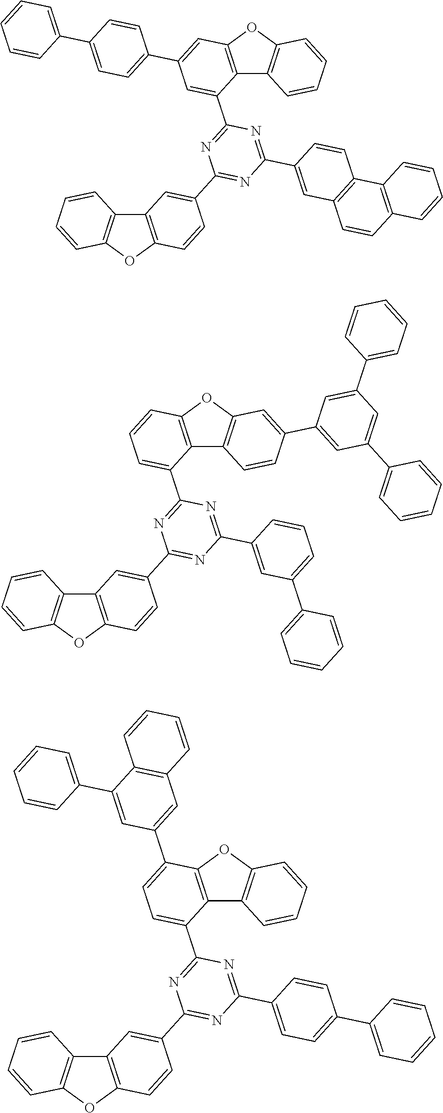 [SMILES: c1ccc(-c2ccc(-c3cc(-c4nc(-c5ccc6c(ccc7ccccc76)c5)nc(-c5ccc6oc7ccccc7c6c5)n4)c4c(c3)oc3ccccc34)cc2)cc1 ; c1ccc(-c2cc(-c3ccccc3)cc(-c3ccc4c(c3)oc3cccc(-c5nc(-c6cccc(-c7ccccc7)c6)nc(-c6ccc7oc8ccccc8c7c6)n5)c34)c2)cc1 ; c1ccc(-c2ccc(-c3nc(-c4ccc5oc6ccccc6c5c4)nc(-c4ccc(-c5cc(-c6ccccc6)c6ccccc6c5)c5oc6ccccc6c45)n3)cc2)cc1]

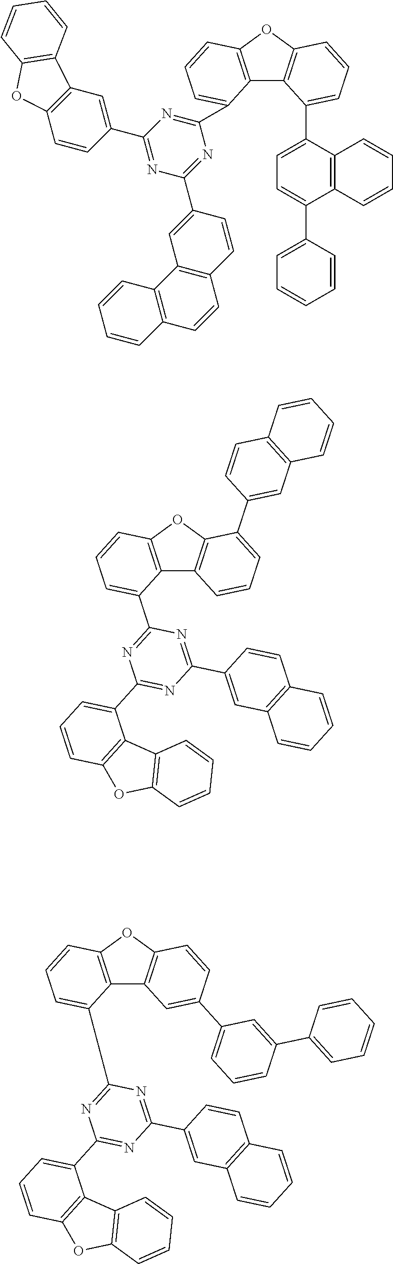 [SMILES: c1ccc(-c2ccc(-c3cccc4oc5cccc(-c6nc(-c7ccc8ccc9ccccc9c8c7)nc(-c7ccc8oc9ccccc9c8c7)n6)c5c34)c3ccccc23)cc1 ; c1ccc2cc(-c3nc(-c4cccc5oc6ccccc6c45)nc(-c4cccc5oc6c(-c7ccc8ccccc8c7)cccc6c45)n3)ccc2c1 ; c1ccc(-c2cccc(-c3ccc4oc5cccc(-c6nc(-c7ccc8ccccc8c7)nc(-c7cccc8oc9ccccc9c78)n6)c5c4c3)c2)cc1]

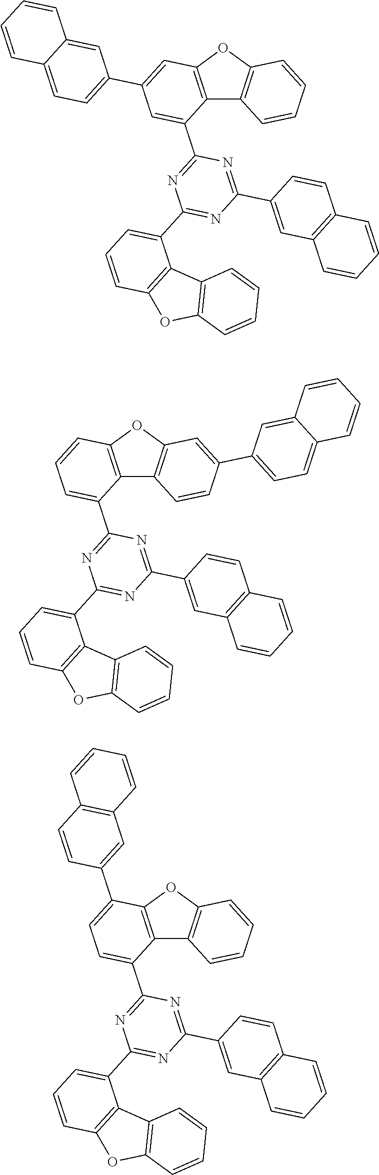

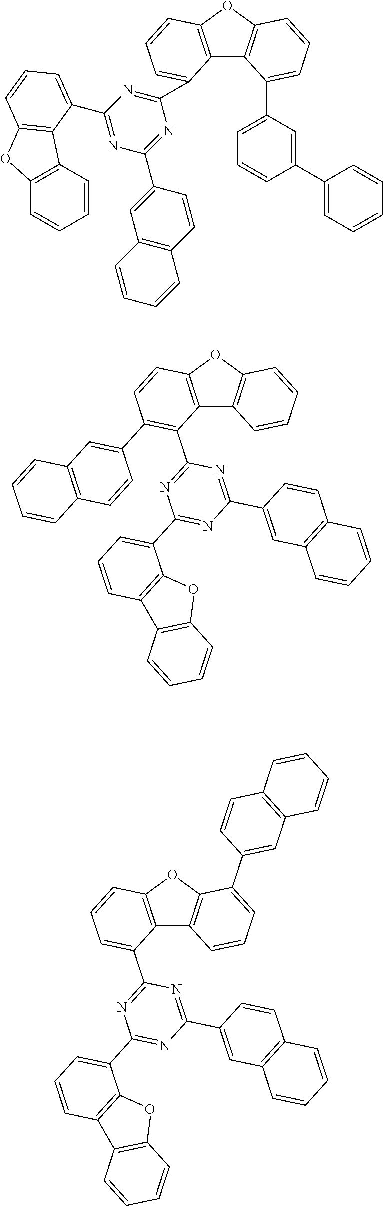 [SMILES: c1ccc(-c2cccc(-c3cccc4oc5cccc(-c6nc(-c7ccc8ccccc8c7)nc(-c7cccc8oc9ccccc9c78)n6)c5c34)c2)cc1 ; c1ccc2cc(-c3nc(-c4cccc5c4oc4ccccc45)nc(-c4c(-c5ccc6ccccc6c5)ccc5oc6ccccc6c45)n3)ccc2c1 ; c1ccc2cc(-c3nc(-c4cccc5c4oc4ccccc45)nc(-c4cccc5oc6c(-c7ccc8ccccc8c7)cccc6c45)n3)ccc2c1]

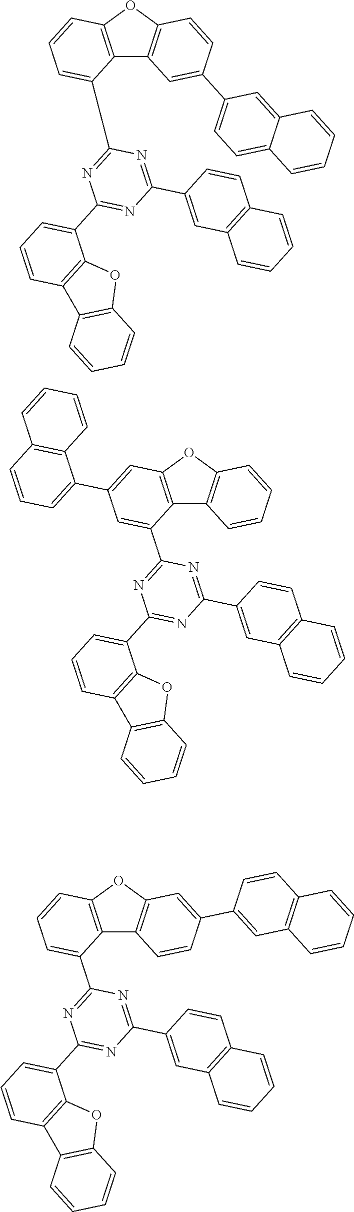

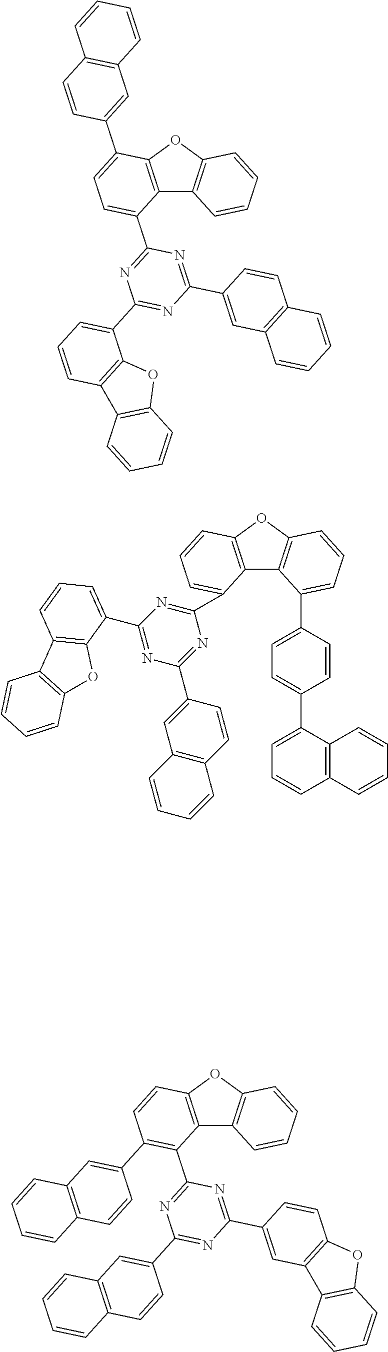 [SMILES: c1ccc2cc(-c3nc(-c4cccc5c4oc4ccccc45)nc(-c4ccc(-c5ccc6ccccc6c5)c5oc6ccccc6c45)n3)ccc2c1 ; c1ccc2cc(-c3nc(-c4cccc5c4oc4ccccc45)nc(-c4cccc5oc6cccc(-c7ccc(-c8cccc9ccccc89)cc7)c6c45)n3)ccc2c1 ; c1ccc2cc(-c3nc(-c4ccc5oc6ccccc6c5c4)nc(-c4c(-c5ccc6ccccc6c5)ccc5oc6ccccc6c45)n3)ccc2c1]

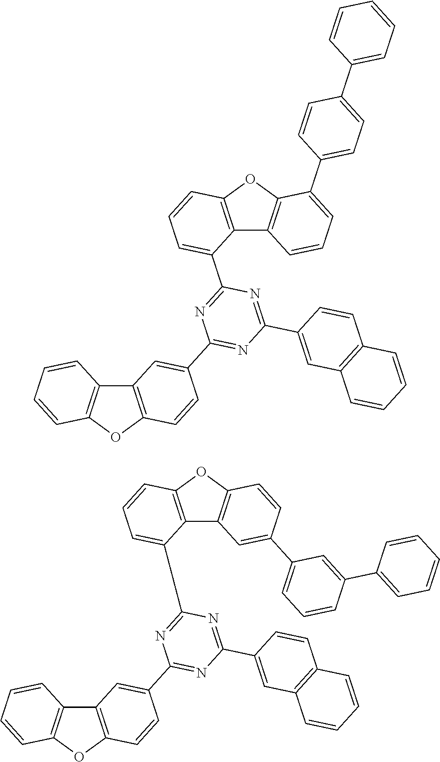 [SMILES: c1ccc(-c2ccc(-c3cccc4c3oc3cccc(-c5nc(-c6ccc7ccccc7c6)nc(-c6ccc7oc8ccccc8c7c6)n5)c34)cc2)cc1 ; c1ccc(-c2cccc(-c3ccc4oc5cccc(-c6nc(-c7ccc8ccccc8c7)nc(-c7ccc8oc9ccccc9c8c7)n6)c5c4c3)c2)cc1]

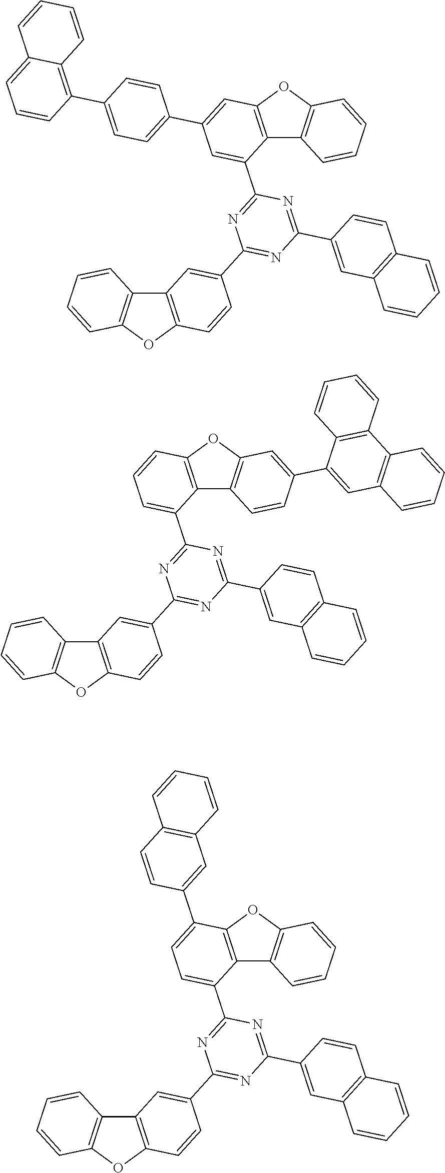 [SMILES: c1ccc2cc(-c3nc(-c4ccc5oc6ccccc6c5c4)nc(-c4cc(-c5ccc(-c6cccc7ccccc67)cc5)cc5oc6ccccc6c45)n3)ccc2c1 ; c1ccc2cc(-c3nc(-c4ccc5oc6ccccc6c5c4)nc(-c4cccc5oc6cc(-c7cc8ccccc8c8ccccc78)ccc6c45)n3)ccc2c1 ; c1ccc2cc(-c3nc(-c4ccc5oc6ccccc6c5c4)nc(-c4ccc(-c5ccc6ccccc6c5)c5oc6ccccc6c45)n3)ccc2c1]

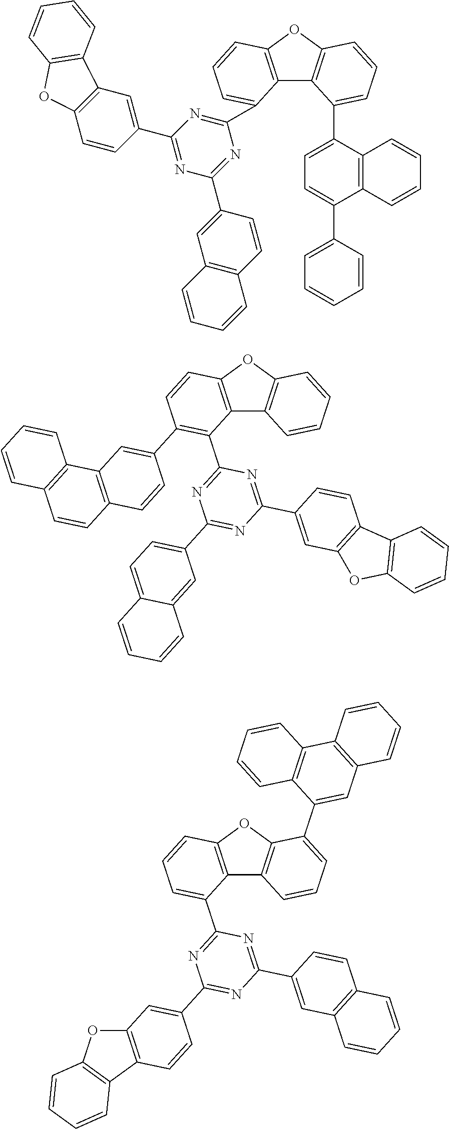

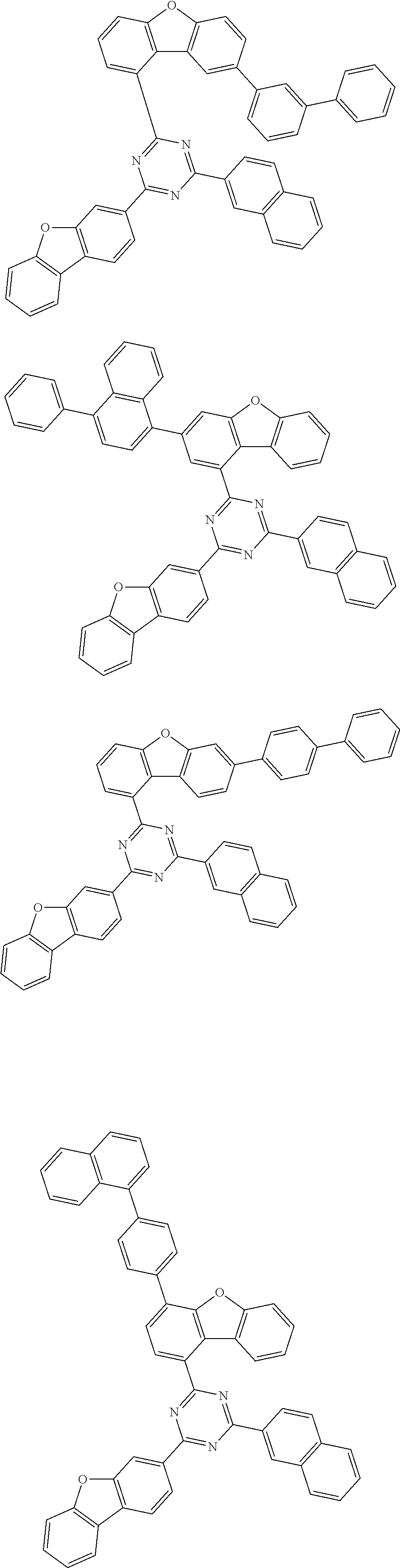 [SMILES: c1ccc(-c2cccc(-c3ccc4oc5cccc(-c6nc(-c7ccc8ccccc8c7)nc(-c7ccc8c(c7)oc7ccccc78)n6)c5c4c3)c2)cc1 ; c1ccc(-c2ccc(-c3cc(-c4nc(-c5ccc6ccccc6c5)nc(-c5ccc6c(c5)oc5ccccc56)n4)c4c(c3)oc3ccccc34)c3ccccc23)cc1 ; c1ccc(-c2ccc(-c3ccc4c(c3)oc3cccc(-c5nc(-c6ccc7ccccc7c6)nc(-c6ccc7c(c6)oc6ccccc67)n5)c34)cc2)cc1 ; c1ccc2cc(-c3nc(-c4ccc5c(c4)oc4ccccc45)nc(-c4ccc(-c5ccc(-c6cccc7ccccc67)cc5)c5oc6ccccc6c45)n3)ccc2c1]

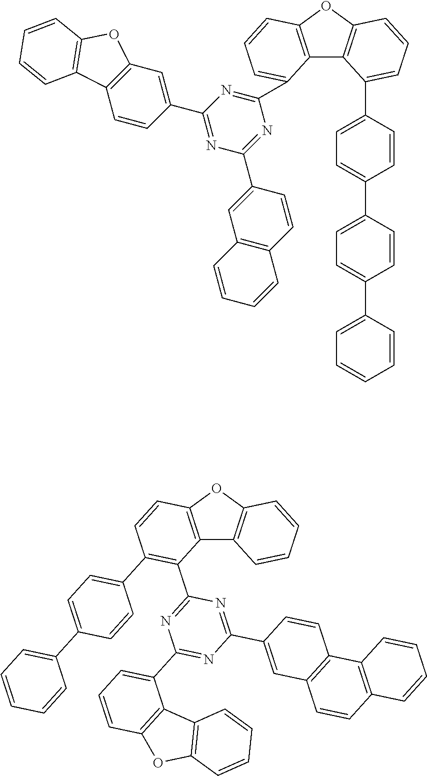 [SMILES: c1ccc(-c2ccc(-c3ccc(-c4cccc5oc6cccc(-c7nc(-c8ccc9ccccc9c8)nc(-c8ccc9c(c8)oc8ccccc89)n7)c6c45)cc3)cc2)cc1 ; c1ccc(-c2ccc(-c3ccc4oc5ccccc5c4c3-c3nc(-c4ccc5c(ccc6ccccc65)c4)nc(-c4cccc5oc6ccccc6c45)n3)cc2)cc1]

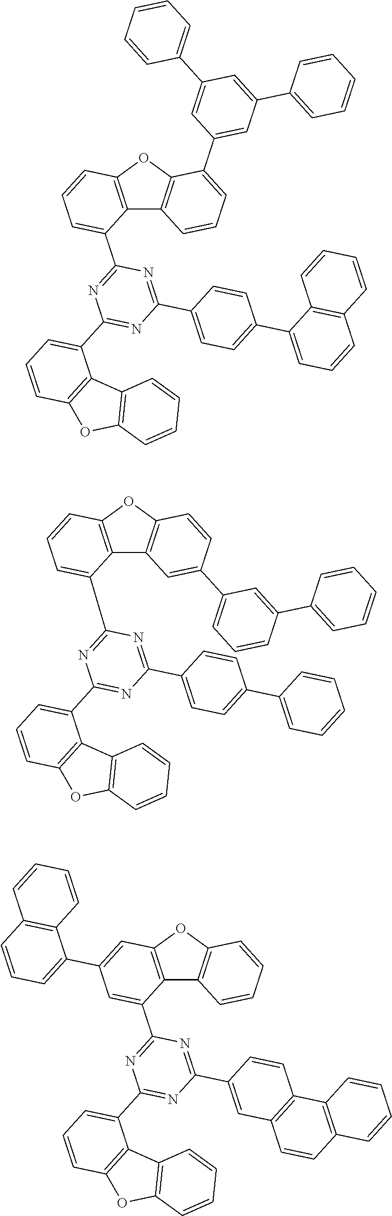 [SMILES: c1ccc(-c2cc(-c3ccccc3)cc(-c3cccc4c3oc3cccc(-c5nc(-c6ccc(-c7cccc8ccccc78)cc6)nc(-c6cccc7oc8ccccc8c67)n5)c34)c2)cc1 ; c1ccc(-c2ccc(-c3nc(-c4cccc5oc6ccccc6c45)nc(-c4cccc5oc6ccc(-c7cccc(-c8ccccc8)c7)cc6c45)n3)cc2)cc1 ; c1ccc2c(-c3cc(-c4nc(-c5ccc6c(ccc7ccccc76)c5)nc(-c5cccc6oc7ccccc7c56)n4)c4c(c3)oc3ccccc34)cccc2c1]

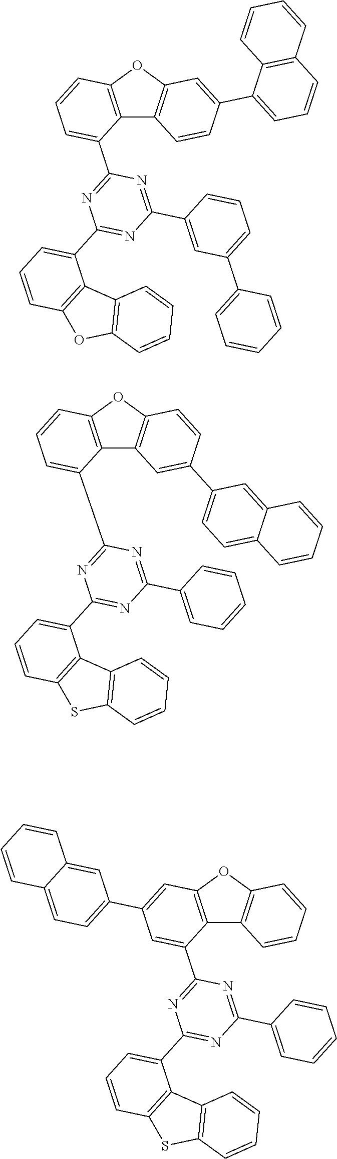 [SMILES: c1ccc(-c2cccc(-c3nc(-c4cccc5oc6ccccc6c45)nc(-c4cccc5oc6cc(-c7cccc8ccccc78)ccc6c45)n3)c2)cc1 ; c1ccc(-c2nc(-c3cccc4oc5ccc(-c6ccc7ccccc7c6)cc5c34)nc(-c3cccc4sc5ccccc5c34)n2)cc1 ; c1ccc(-c2nc(-c3cc(-c4ccc5ccccc5c4)cc4oc5ccccc5c34)nc(-c3cccc4sc5ccccc5c34)n2)cc1]

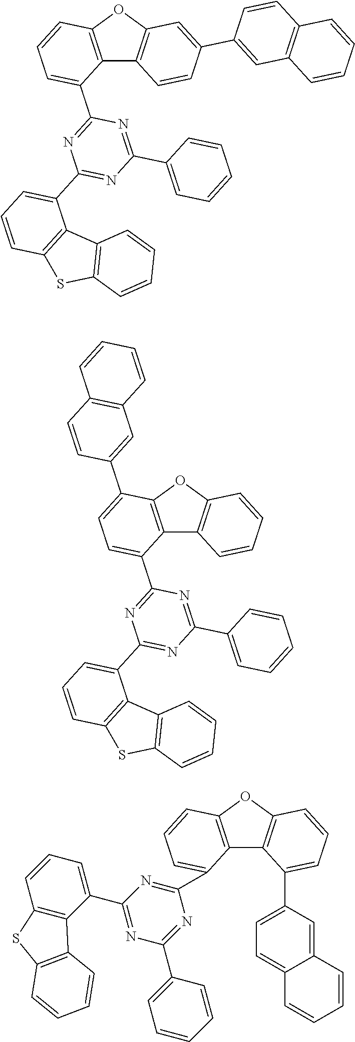

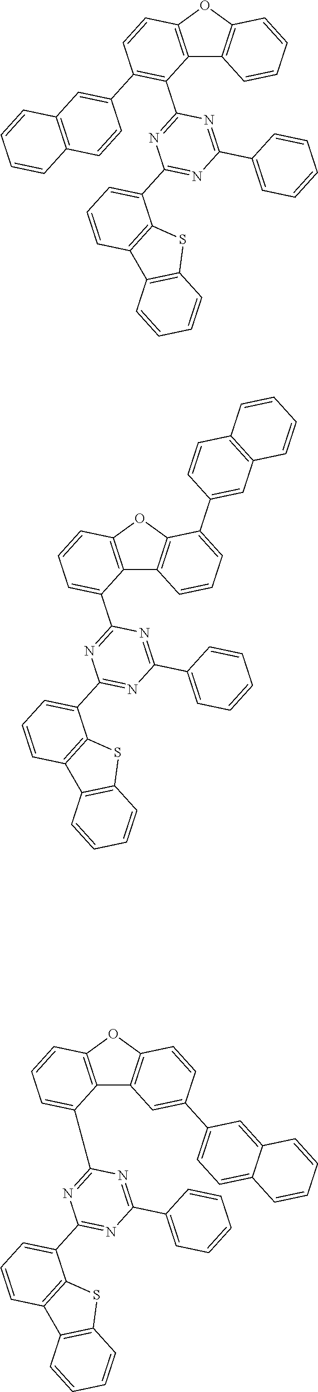 [SMILES: c1ccc(-c2nc(-c3cccc4c3sc3ccccc34)nc(-c3c(-c4ccc5ccccc5c4)ccc4oc5ccccc5c34)n2)cc1 ; c1ccc(-c2nc(-c3cccc4c3sc3ccccc34)nc(-c3cccc4oc5c(-c6ccc7ccccc7c6)cccc5c34)n2)cc1 ; c1ccc(-c2nc(-c3cccc4c3sc3ccccc34)nc(-c3cccc4oc5ccc(-c6ccc7ccccc7c6)cc5c34)n2)cc1]

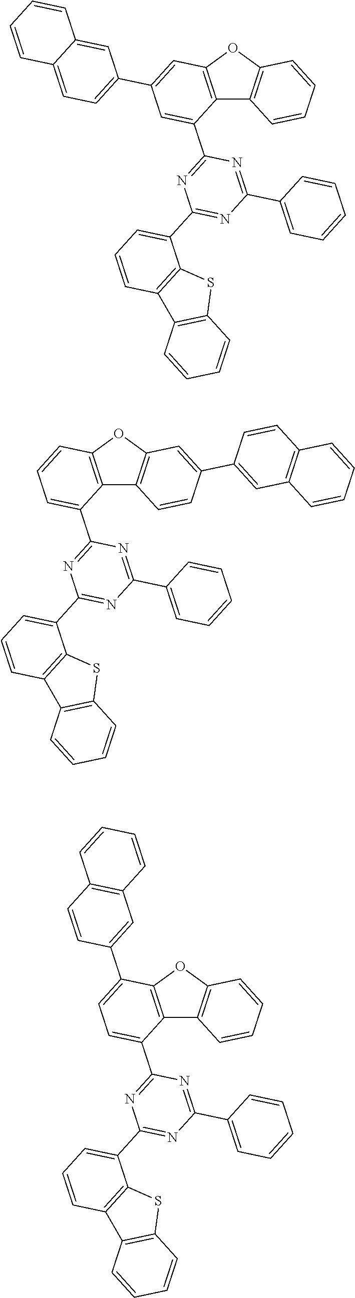 [SMILES: c1ccc(-c2nc(-c3cccc4c3sc3ccccc34)nc(-c3cc(-c4ccc5ccccc5c4)cc4oc5ccccc5c34)n2)cc1 ; c1ccc(-c2nc(-c3cccc4c3sc3ccccc34)nc(-c3cccc4oc5cc(-c6ccc7ccccc7c6)ccc5c34)n2)cc1 ; c1ccc(-c2nc(-c3cccc4c3sc3ccccc34)nc(-c3ccc(-c4ccc5ccccc5c4)c4oc5ccccc5c34)n2)cc1]

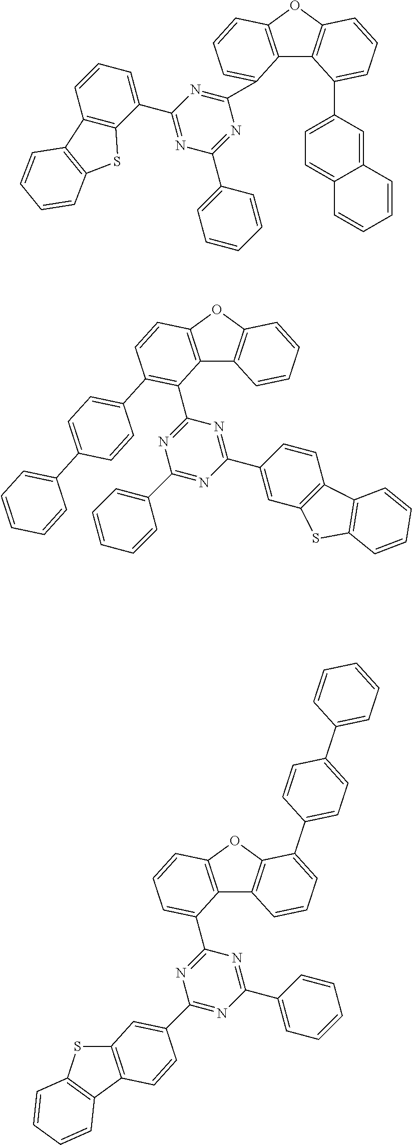 [SMILES: c1ccc(-c2nc(-c3cccc4c3sc3ccccc34)nc(-c3cccc4oc5cccc(-c6ccc7ccccc7c6)c5c34)n2)cc1 ; c1ccc(-c2ccc(-c3ccc4oc5ccccc5c4c3-c3nc(-c4ccccc4)nc(-c4ccc5c(c4)sc4ccccc45)n3)cc2)cc1 ; c1ccc(-c2ccc(-c3cccc4c3oc3cccc(-c5nc(-c6ccccc6)nc(-c6ccc7c(c6)sc6ccccc67)n5)c34)cc2)cc1]

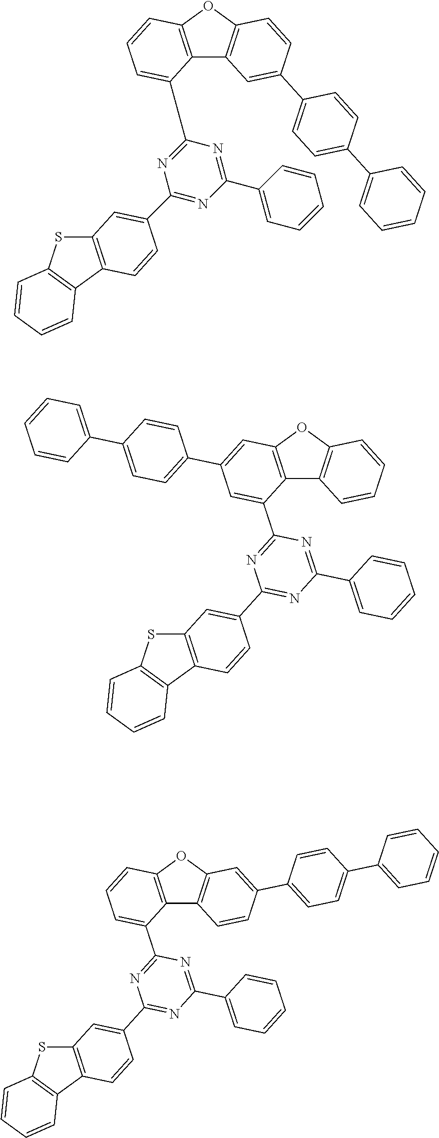 [SMILES: c1ccc(-c2ccc(-c3ccc4oc5cccc(-c6nc(-c7ccccc7)nc(-c7ccc8c(c7)sc7ccccc78)n6)c5c4c3)cc2)cc1 ; c1ccc(-c2ccc(-c3cc(-c4nc(-c5ccccc5)nc(-c5ccc6c(c5)sc5ccccc56)n4)c4c(c3)oc3ccccc34)cc2)cc1 ; c1ccc(-c2ccc(-c3ccc4c(c3)oc3cccc(-c5nc(-c6ccccc6)nc(-c6ccc7c(c6)sc6ccccc67)n5)c34)cc2)cc1]

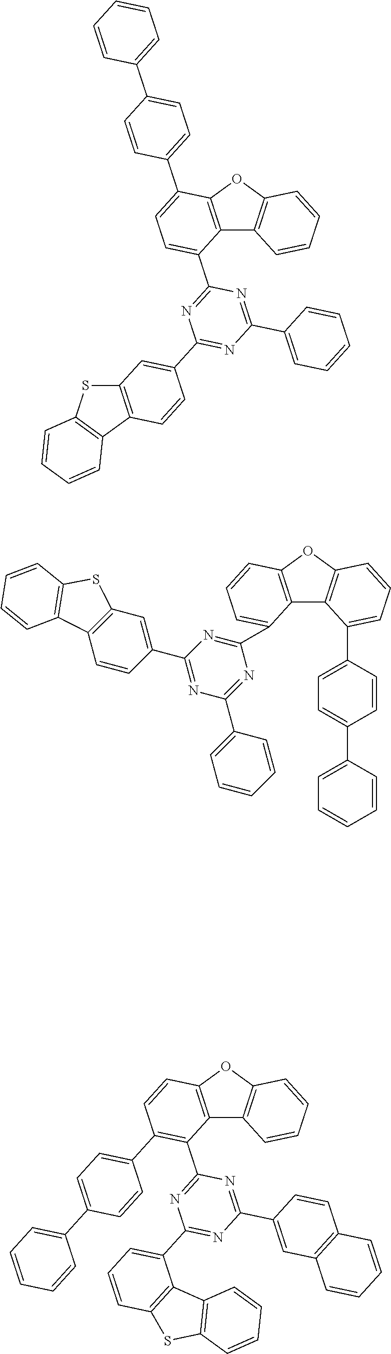 [SMILES: c1ccc(-c2ccc(-c3ccc(-c4nc(-c5ccccc5)nc(-c5ccc6c(c5)sc5ccccc56)n4)c4c3oc3ccccc34)cc2)cc1 ; c1ccc(-c2ccc(-c3cccc4oc5cccc(-c6nc(-c7ccccc7)nc(-c7ccc8c(c7)sc7ccccc78)n6)c5c34)cc2)cc1 ; c1ccc(-c2ccc(-c3ccc4oc5ccccc5c4c3-c3nc(-c4ccc5ccccc5c4)nc(-c4cccc5sc6ccccc6c45)n3)cc2)cc1]

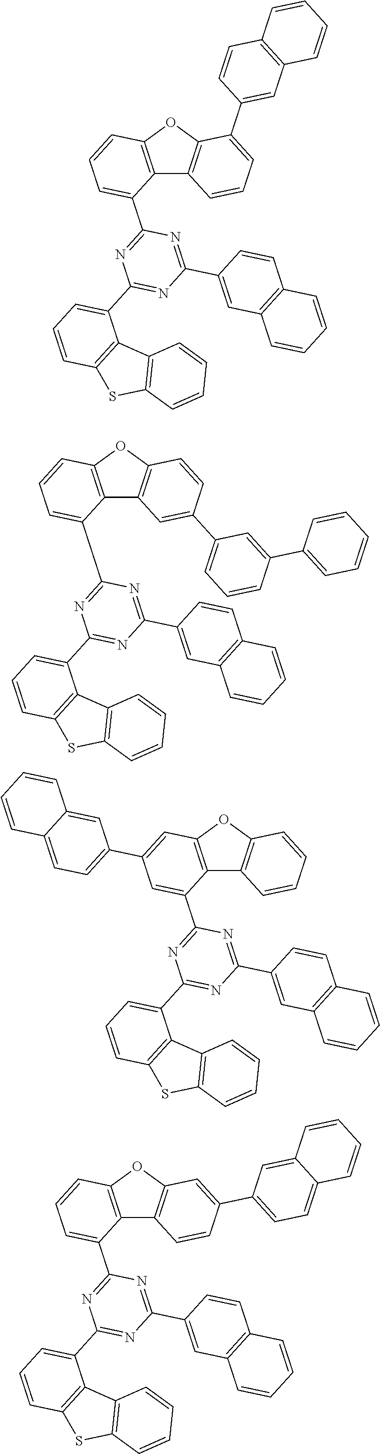

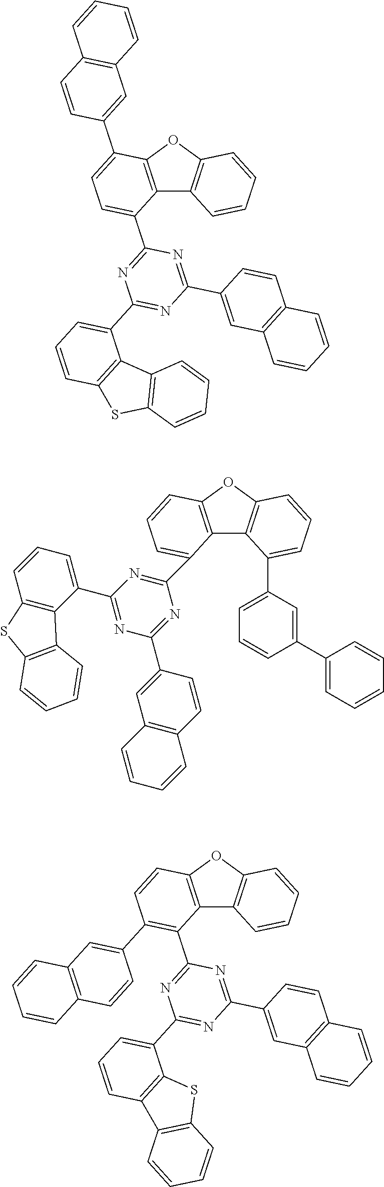 [SMILES: c1ccc2cc(-c3nc(-c4cccc5sc6ccccc6c45)nc(-c4ccc(-c5ccc6ccccc6c5)c5oc6ccccc6c45)n3)ccc2c1 ; c1ccc(-c2cccc(-c3cccc4oc5cccc(-c6nc(-c7ccc8ccccc8c7)nc(-c7cccc8sc9ccccc9c78)n6)c5c34)c2)cc1 ; c1ccc2cc(-c3nc(-c4cccc5c4sc4ccccc45)nc(-c4c(-c5ccc6ccccc6c5)ccc5oc6ccccc6c45)n3)ccc2c1]

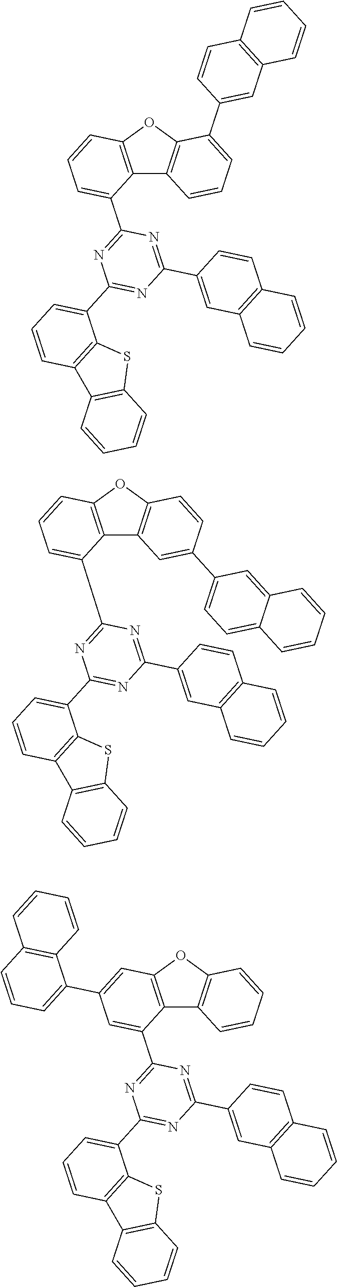

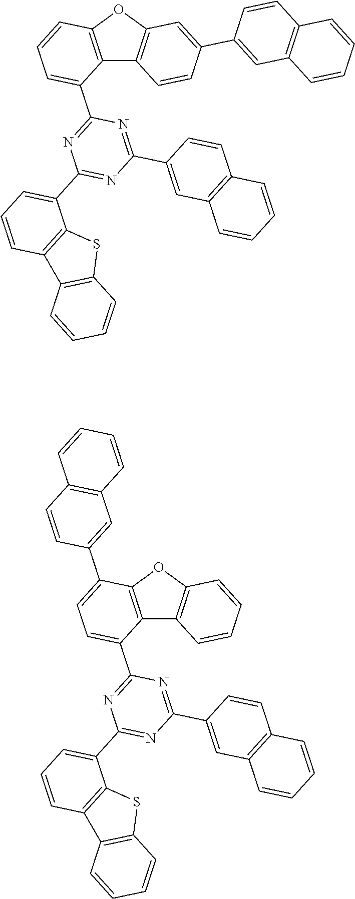 [SMILES: c1ccc2cc(-c3ccc4c(c3)oc3cccc(-c5nc(-c6ccc7ccccc7c6)nc(-c6cccc7c6sc6ccccc67)n5)c34)ccc2c1 ; c1ccc2cc(-c3nc(-c4cccc5c4sc4ccccc45)nc(-c4ccc(-c5ccc6ccccc6c5)c5oc6ccccc6c45)n3)ccc2c1]

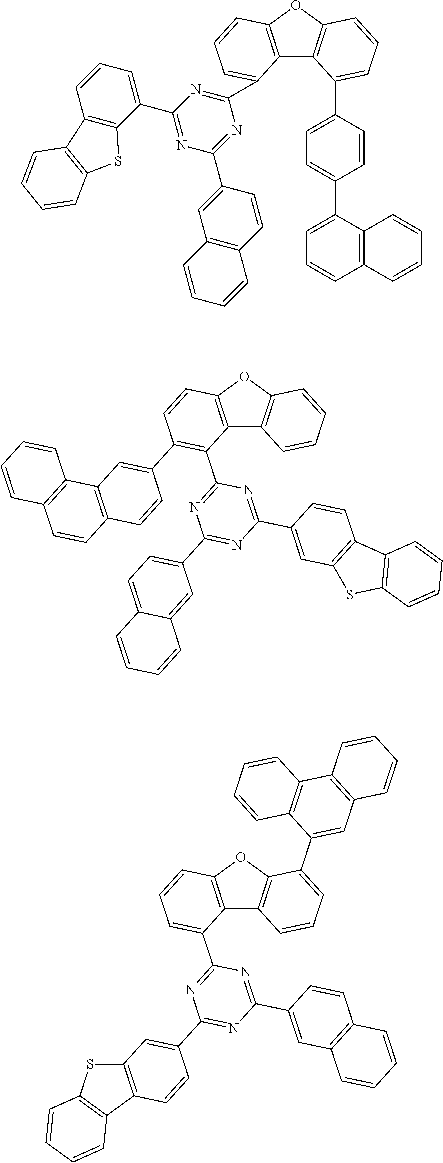 [SMILES: c1ccc2cc(-c3nc(-c4cccc5c4sc4ccccc45)nc(-c4cccc5oc6cccc(-c7ccc(-c8cccc9ccccc89)cc7)c6c45)n3)ccc2c1 ; c1ccc2cc(-c3nc(-c4ccc5c(c4)sc4ccccc45)nc(-c4c(-c5ccc6ccc7ccccc7c6c5)ccc5oc6ccccc6c45)n3)ccc2c1 ; c1ccc2cc(-c3nc(-c4ccc5c(c4)sc4ccccc45)nc(-c4cccc5oc6c(-c7cc8ccccc8c8ccccc78)cccc6c45)n3)ccc2c1]

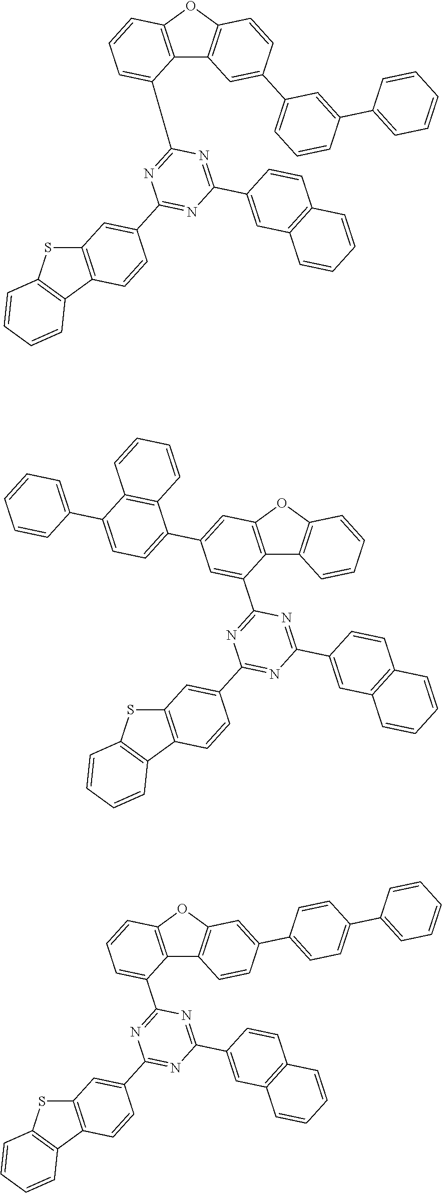 [SMILES: c1ccc(-c2cccc(-c3ccc4oc5cccc(-c6nc(-c7ccc8ccccc8c7)nc(-c7ccc8c(c7)sc7ccccc78)n6)c5c4c3)c2)cc1 ; c1ccc(-c2ccc(-c3cc(-c4nc(-c5ccc6ccccc6c5)nc(-c5ccc6c(c5)sc5ccccc56)n4)c4c(c3)oc3ccccc34)c3ccccc23)cc1 ; c1ccc(-c2ccc(-c3ccc4c(c3)oc3cccc(-c5nc(-c6ccc7ccccc7c6)nc(-c6ccc7c(c6)sc6ccccc67)n5)c34)cc2)cc1]

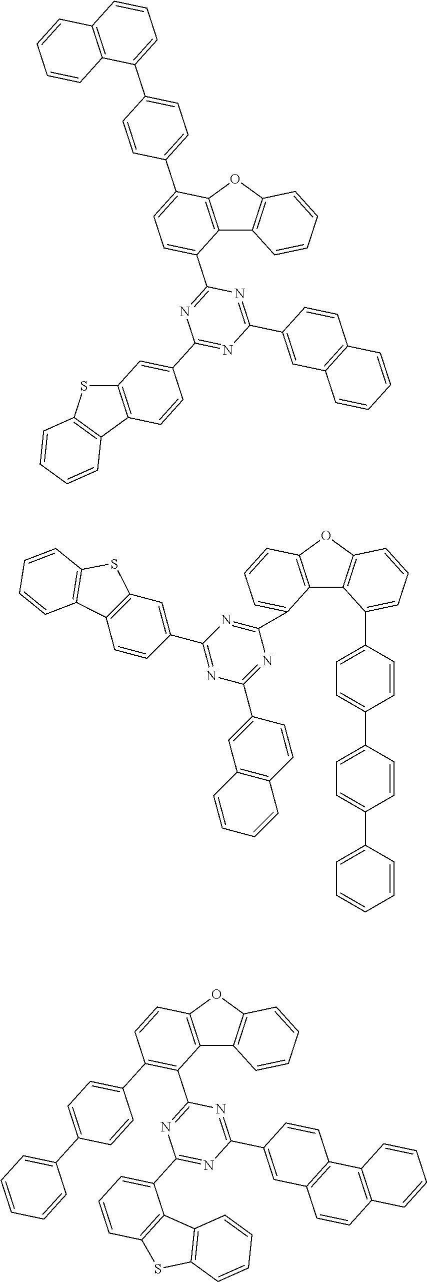 [SMILES: c1ccc2cc(-c3nc(-c4ccc5c(c4)sc4ccccc45)nc(-c4ccc(-c5ccc(-c6cccc7ccccc67)cc5)c5oc6ccccc6c45)n3)ccc2c1 ; c1ccc(-c2ccc(-c3ccc(-c4cccc5oc6cccc(-c7nc(-c8ccc9ccccc9c8)nc(-c8ccc9c(c8)sc8ccccc89)n7)c6c45)cc3)cc2)cc1 ; c1ccc(-c2ccc(-c3ccc4oc5ccccc5c4c3-c3nc(-c4ccc5c(ccc6ccccc65)c4)nc(-c4cccc5sc6ccccc6c45)n3)cc2)cc1]

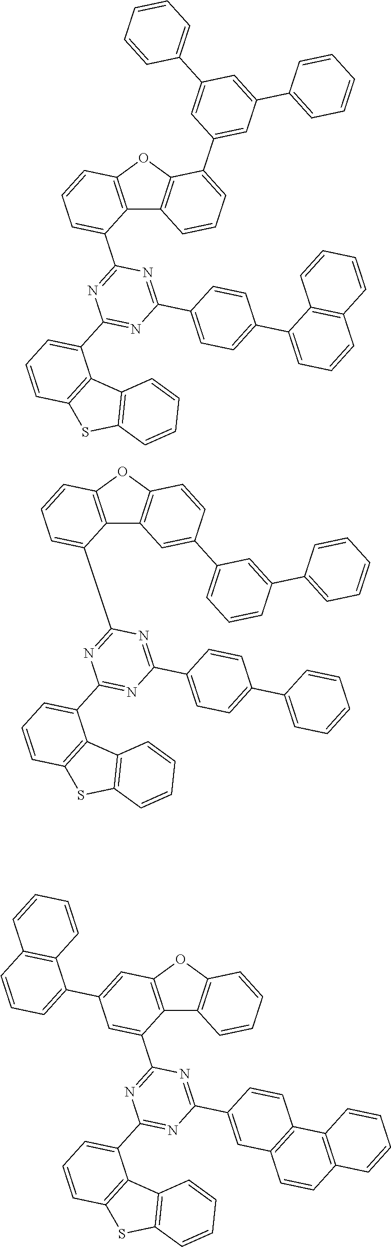

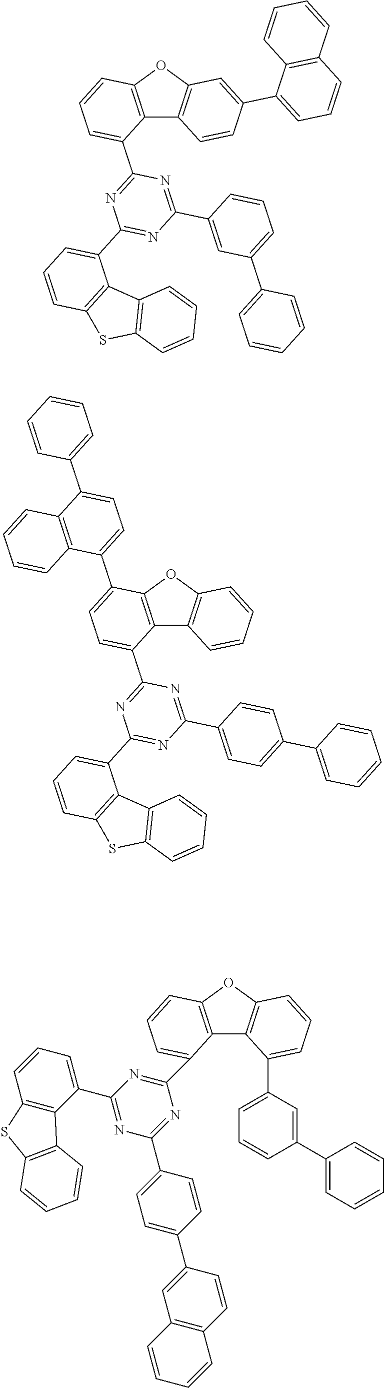 [SMILES: c1ccc(-c2cccc(-c3nc(-c4cccc5oc6cc(-c7cccc8ccccc78)ccc6c45)nc(-c4cccc5sc6ccccc6c45)n3)c2)cc1 ; c1ccc(-c2ccc(-c3nc(-c4cccc5sc6ccccc6c45)nc(-c4ccc(-c5ccc(-c6ccccc6)c6ccccc56)c5oc6ccccc6c45)n3)cc2)cc1 ; c1ccc(-c2cccc(-c3cccc4oc5cccc(-c6nc(-c7ccc(-c8ccc9ccccc9c8)cc7)nc(-c7cccc8sc9ccccc9c78)n6)c5c34)c2)cc1]

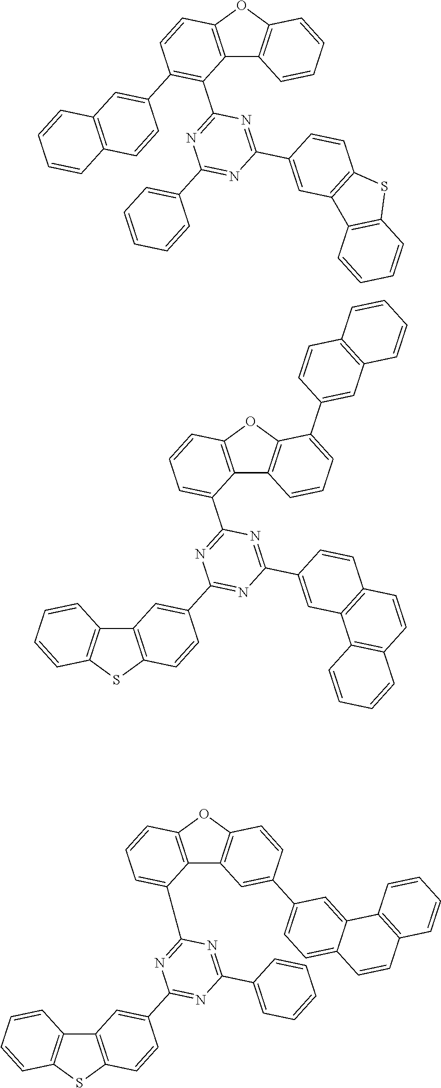

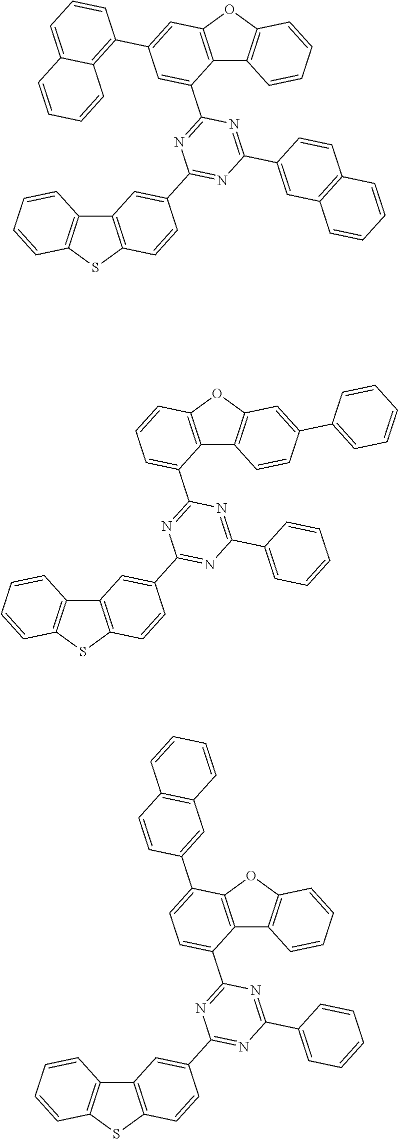 [SMILES: c1ccc2cc(-c3nc(-c4ccc5sc6ccccc6c5c4)nc(-c4cc(-c5cccc6ccccc56)cc5oc6ccccc6c45)n3)ccc2c1 ; c1ccc(-c2ccc3c(c2)oc2cccc(-c4nc(-c5ccccc5)nc(-c5ccc6sc7ccccc7c6c5)n4)c23)cc1 ; c1ccc(-c2nc(-c3ccc4sc5ccccc5c4c3)nc(-c3ccc(-c4ccc5ccccc5c4)c4oc5ccccc5c34)n2)cc1]

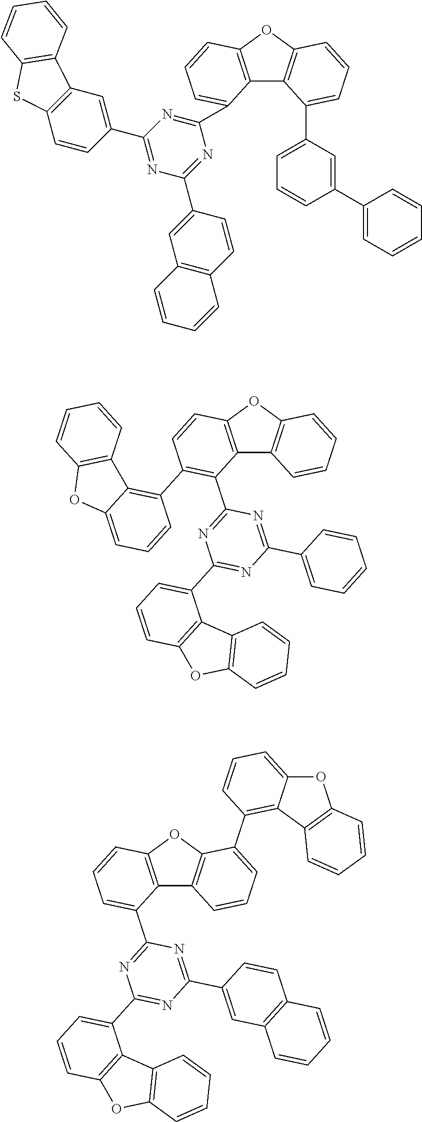 [SMILES: c1ccc(-c2cccc(-c3cccc4oc5cccc(-c6nc(-c7ccc8ccccc8c7)nc(-c7ccc8sc9ccccc9c8c7)n6)c5c34)c2)cc1 ; c1ccc(-c2nc(-c3cccc4oc5ccccc5c34)nc(-c3c(-c4cccc5oc6ccccc6c45)ccc4oc5ccccc5c34)n2)cc1 ; c1ccc2cc(-c3nc(-c4cccc5oc6ccccc6c45)nc(-c4cccc5oc6c(-c7cccc8oc9ccccc9c78)cccc6c45)n3)ccc2c1]

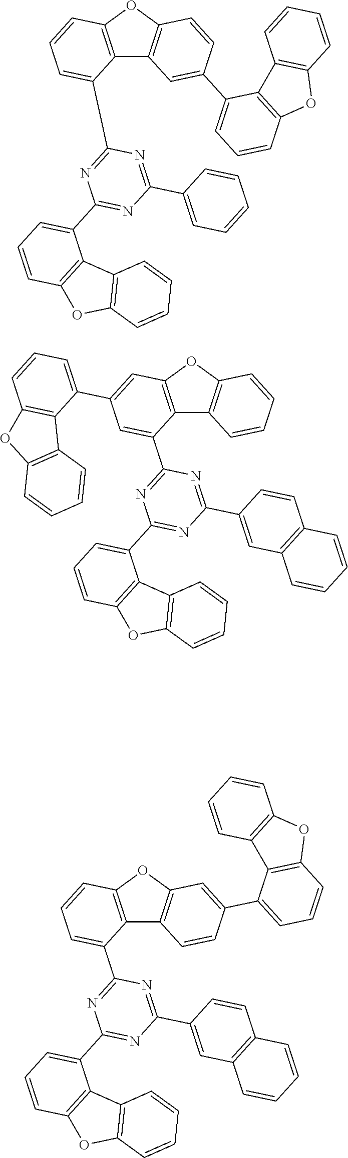

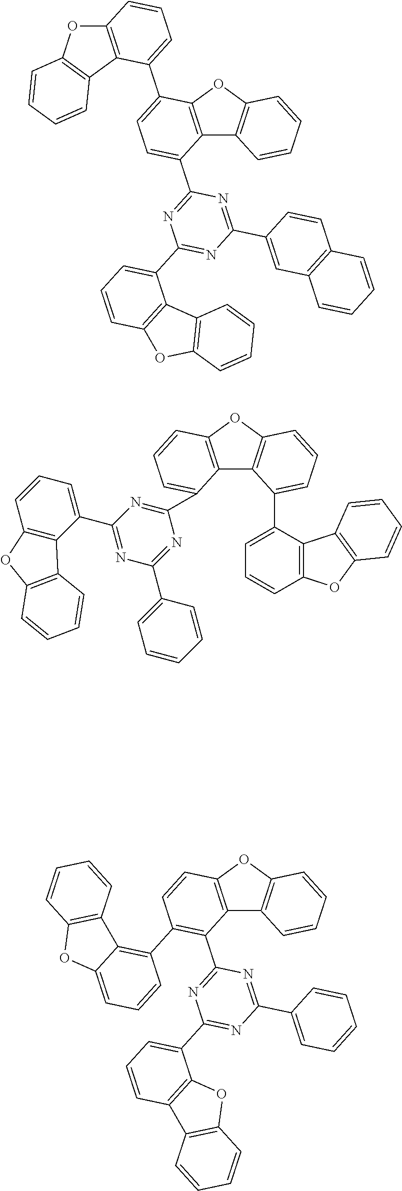 [SMILES: c1ccc2cc(-c3nc(-c4cccc5oc6ccccc6c45)nc(-c4ccc(-c5cccc6oc7ccccc7c56)c5oc6ccccc6c45)n3)ccc2c1 ; c1ccc(-c2nc(-c3cccc4oc5ccccc5c34)nc(-c3cccc4oc5cccc(-c6cccc7oc8ccccc8c67)c5c34)n2)cc1 ; c1ccc(-c2nc(-c3cccc4c3oc3ccccc34)nc(-c3c(-c4cccc5oc6ccccc6c45)ccc4oc5ccccc5c34)n2)cc1]

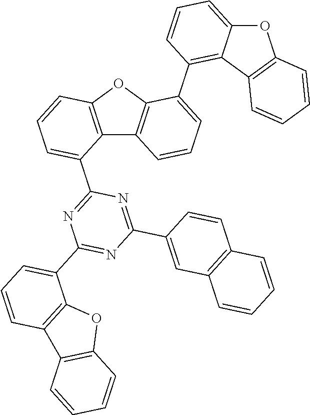 [SMILES: c1ccc2cc(-c3nc(-c4cccc5c4oc4ccccc45)nc(-c4cccc5oc6c(-c7cccc8oc9ccccc9c78)cccc6c45)n3)ccc2c1]

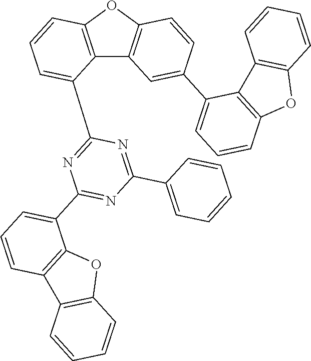 [SMILES: c1ccc(-c2nc(-c3cccc4c3oc3ccccc34)nc(-c3cccc4oc5ccc(-c6cccc7oc8ccccc8c67)cc5c34)n2)cc1]

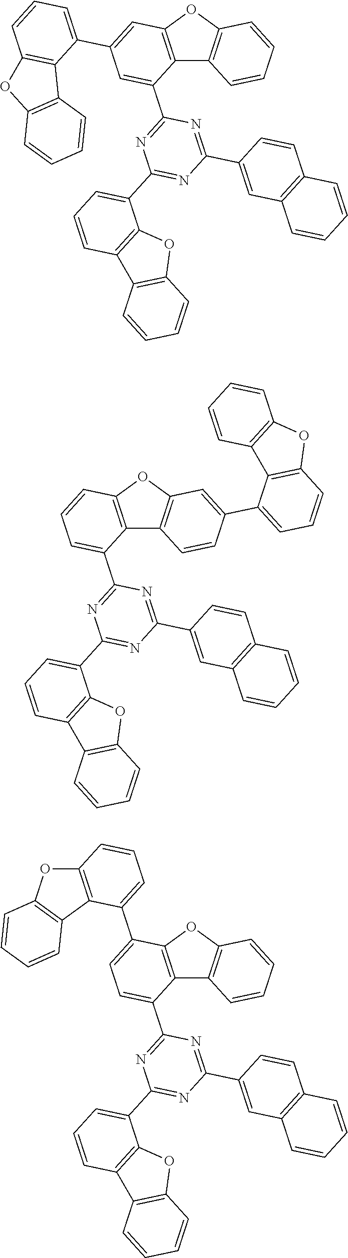

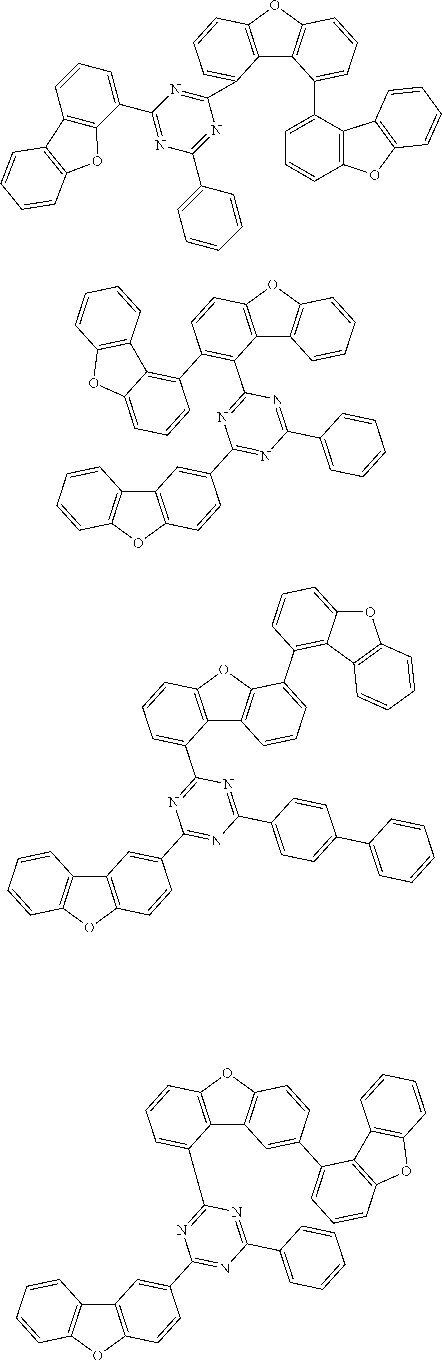 [SMILES: c1ccc(-c2nc(-c3cccc4c3oc3ccccc34)nc(-c3cccc4oc5cccc(-c6cccc7oc8ccccc8c67)c5c34)n2)cc1 ; c1ccc(-c2nc(-c3ccc4oc5ccccc5c4c3)nc(-c3c(-c4cccc5oc6ccccc6c45)ccc4oc5ccccc5c34)n2)cc1 ; c1ccc(-c2ccc(-c3nc(-c4ccc5oc6ccccc6c5c4)nc(-c4cccc5oc6c(-c7cccc8oc9ccccc9c78)cccc6c45)n3)cc2)cc1 ; c1ccc(-c2nc(-c3ccc4oc5ccccc5c4c3)nc(-c3cccc4oc5ccc(-c6cccc7oc8ccccc8c67)cc5c34)n2)cc1]

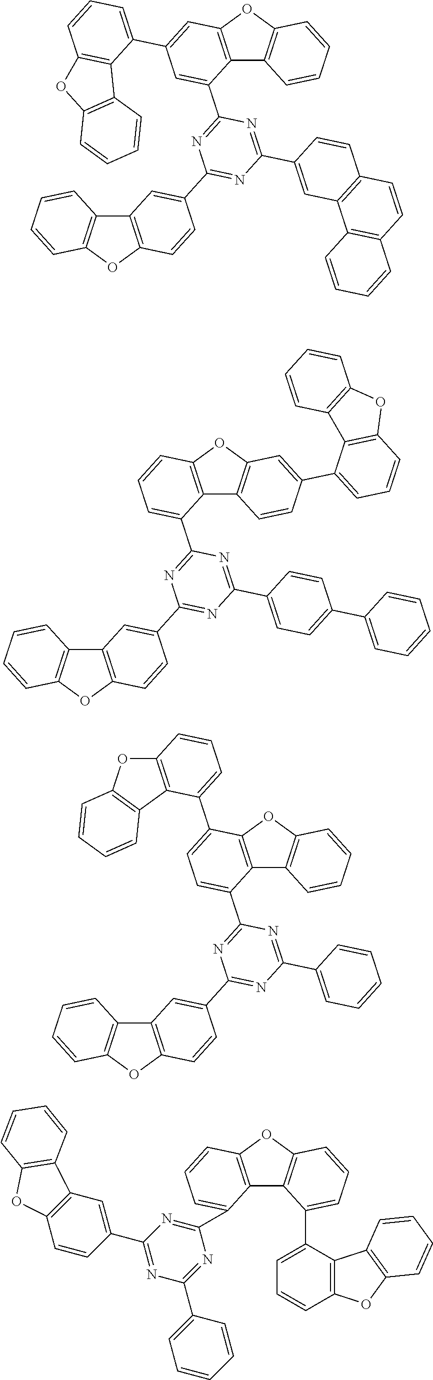 [SMILES: c1ccc2c(c1)ccc1ccc(-c3nc(-c4ccc5oc6ccccc6c5c4)nc(-c4cc(-c5cccc6oc7ccccc7c56)cc5oc6ccccc6c45)n3)cc12 ; c1ccc(-c2ccc(-c3nc(-c4ccc5oc6ccccc6c5c4)nc(-c4cccc5oc6cc(-c7cccc8oc9ccccc9c78)ccc6c45)n3)cc2)cc1 ; c1ccc(-c2nc(-c3ccc4oc5ccccc5c4c3)nc(-c3ccc(-c4cccc5oc6ccccc6c45)c4oc5ccccc5c34)n2)cc1 ; c1ccc(-c2nc(-c3ccc4oc5ccccc5c4c3)nc(-c3cccc4oc5cccc(-c6cccc7oc8ccccc8c67)c5c34)n2)cc1]

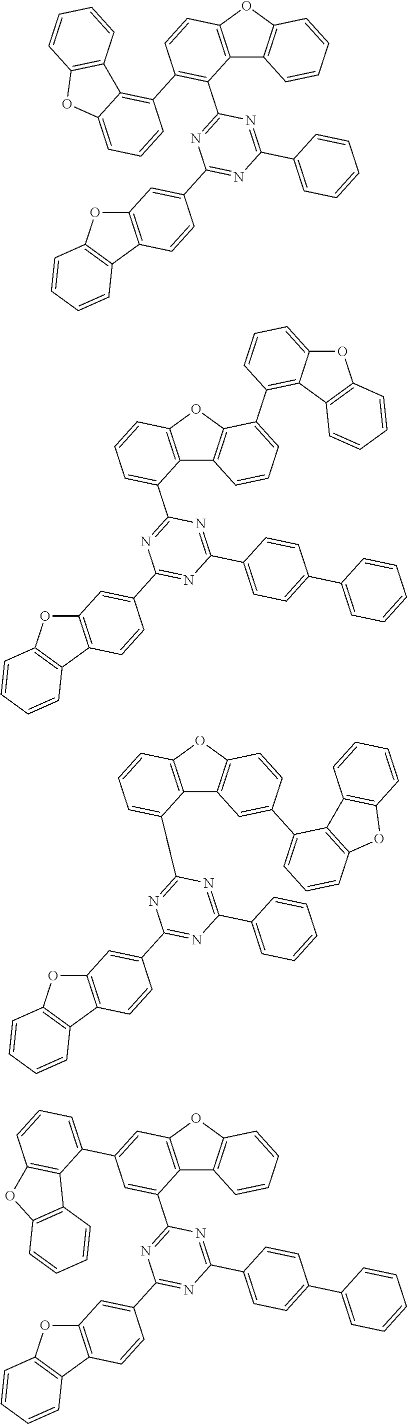

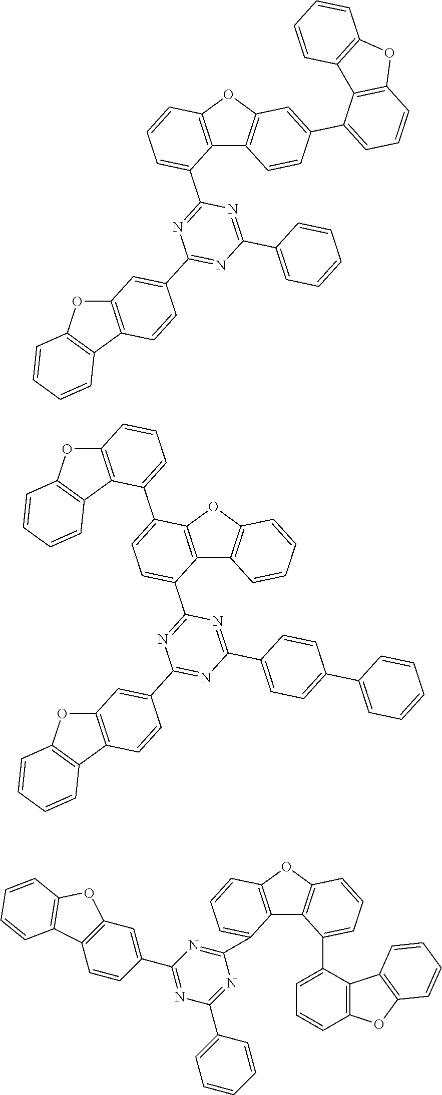 [SMILES: c1ccc(-c2nc(-c3ccc4c(c3)oc3ccccc34)nc(-c3cccc4oc5cc(-c6cccc7oc8ccccc8c67)ccc5c34)n2)cc1 ; c1ccc(-c2ccc(-c3nc(-c4ccc5c(c4)oc4ccccc45)nc(-c4ccc(-c5cccc6oc7ccccc7c56)c5oc6ccccc6c45)n3)cc2)cc1 ; c1ccc(-c2nc(-c3ccc4c(c3)oc3ccccc34)nc(-c3cccc4oc5cccc(-c6cccc7oc8ccccc8c67)c5c34)n2)cc1]

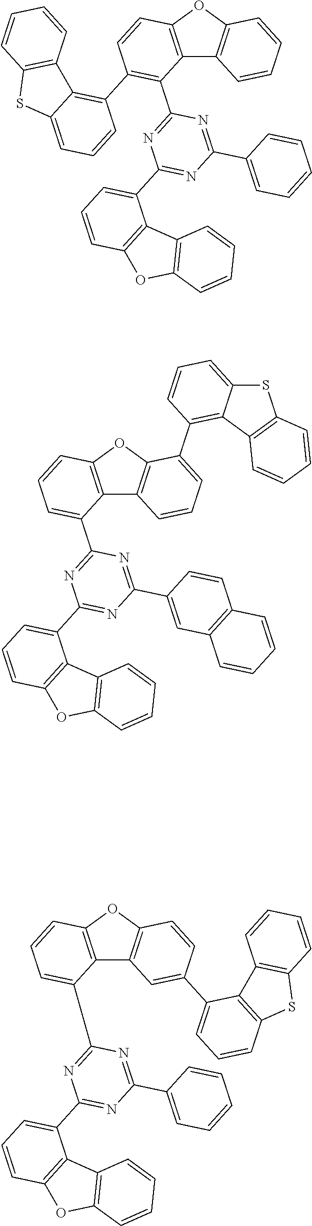 [SMILES: c1ccc(-c2nc(-c3cccc4oc5ccccc5c34)nc(-c3c(-c4cccc5sc6ccccc6c45)ccc4oc5ccccc5c34)n2)cc1 ; c1ccc2cc(-c3nc(-c4cccc5oc6ccccc6c45)nc(-c4cccc5oc6c(-c7cccc8sc9ccccc9c78)cccc6c45)n3)ccc2c1 ; c1ccc(-c2nc(-c3cccc4oc5ccccc5c34)nc(-c3cccc4oc5ccc(-c6cccc7sc8ccccc8c67)cc5c34)n2)cc1]

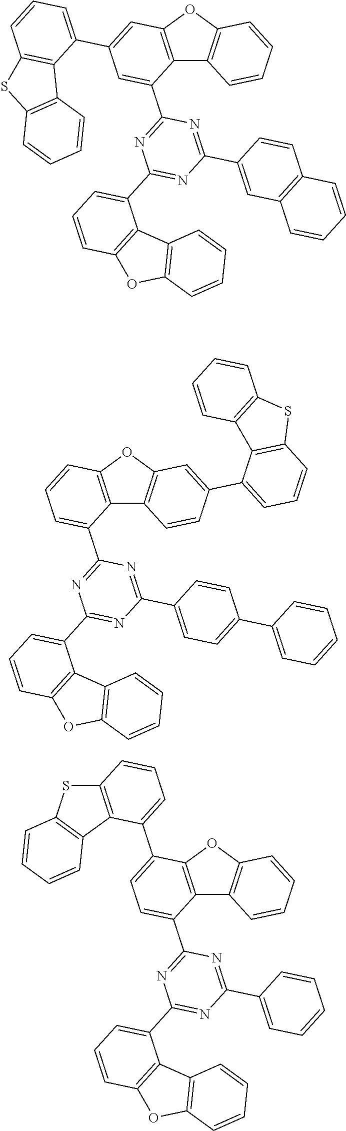

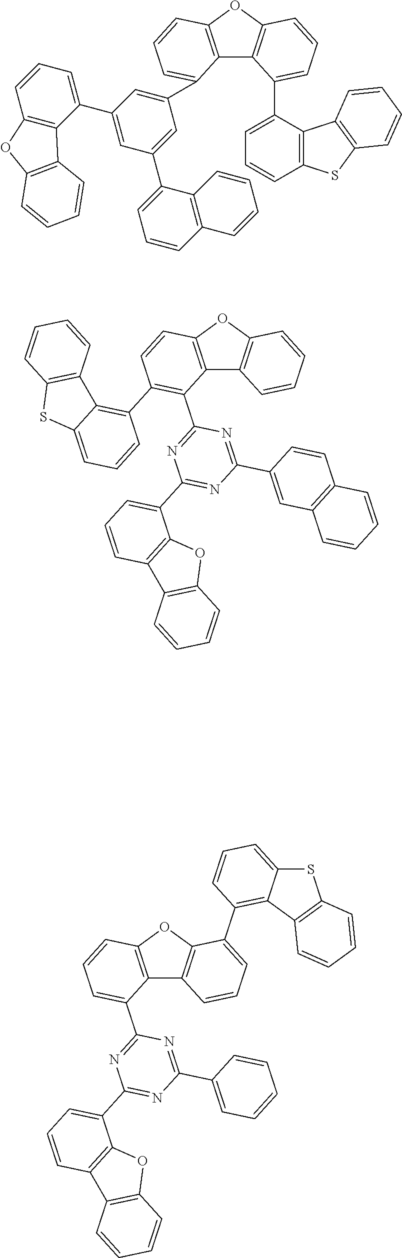 [SMILES: c1ccc2c(-c3cc(-c4cccc5oc6ccccc6c45)cc(-c4cccc5oc6cccc(-c7cccc8sc9ccccc9c78)c6c45)c3)cccc2c1 ; c1ccc2cc(-c3nc(-c4cccc5c4oc4ccccc45)nc(-c4c(-c5cccc6sc7ccccc7c56)ccc5oc6ccccc6c45)n3)ccc2c1 ; c1ccc(-c2nc(-c3cccc4c3oc3ccccc34)nc(-c3cccc4oc5c(-c6cccc7sc8ccccc8c67)cccc5c34)n2)cc1]

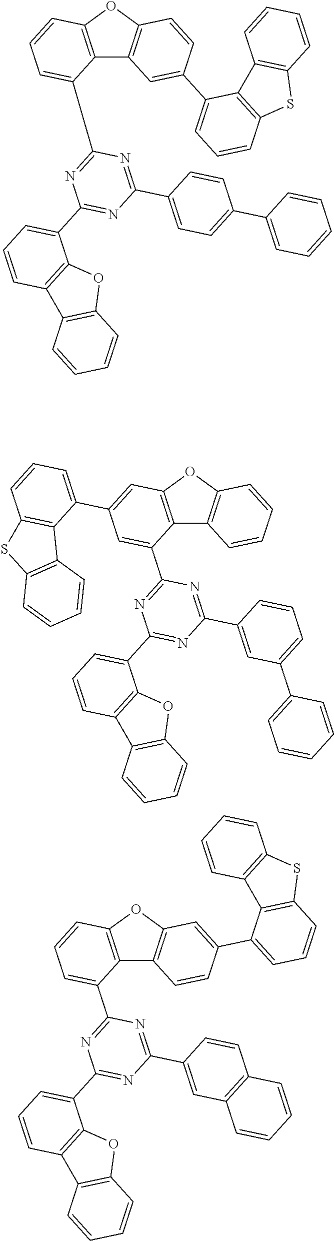 [SMILES: c1ccc(-c2ccc(-c3nc(-c4cccc5c4oc4ccccc45)nc(-c4cccc5oc6ccc(-c7cccc8sc9ccccc9c78)cc6c45)n3)cc2)cc1 ; c1ccc(-c2cccc(-c3nc(-c4cccc5c4oc4ccccc45)nc(-c4cc(-c5cccc6sc7ccccc7c56)cc5oc6ccccc6c45)n3)c2)cc1 ; c1ccc2cc(-c3nc(-c4cccc5c4oc4ccccc45)nc(-c4cccc5oc6cc(-c7cccc8sc9ccccc9c78)ccc6c45)n3)ccc2c1]

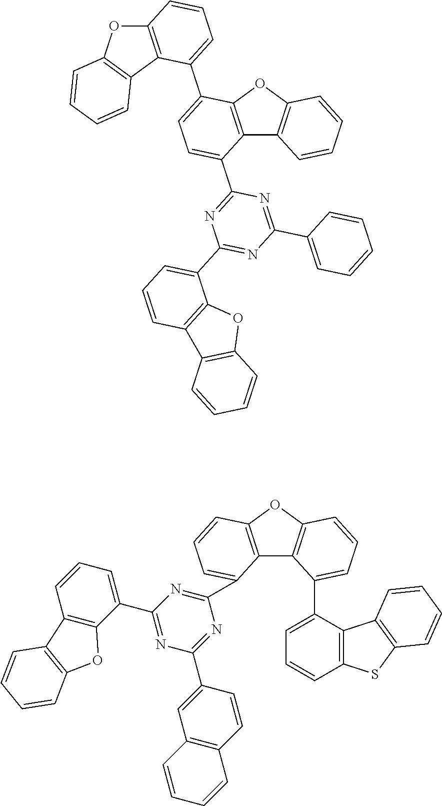 [SMILES: c1ccc(-c2nc(-c3cccc4c3oc3ccccc34)nc(-c3ccc(-c4cccc5oc6ccccc6c45)c4oc5ccccc5c34)n2)cc1 ; c1ccc2cc(-c3nc(-c4cccc5c4oc4ccccc45)nc(-c4cccc5oc6cccc(-c7cccc8sc9ccccc9c78)c6c45)n3)ccc2c1]

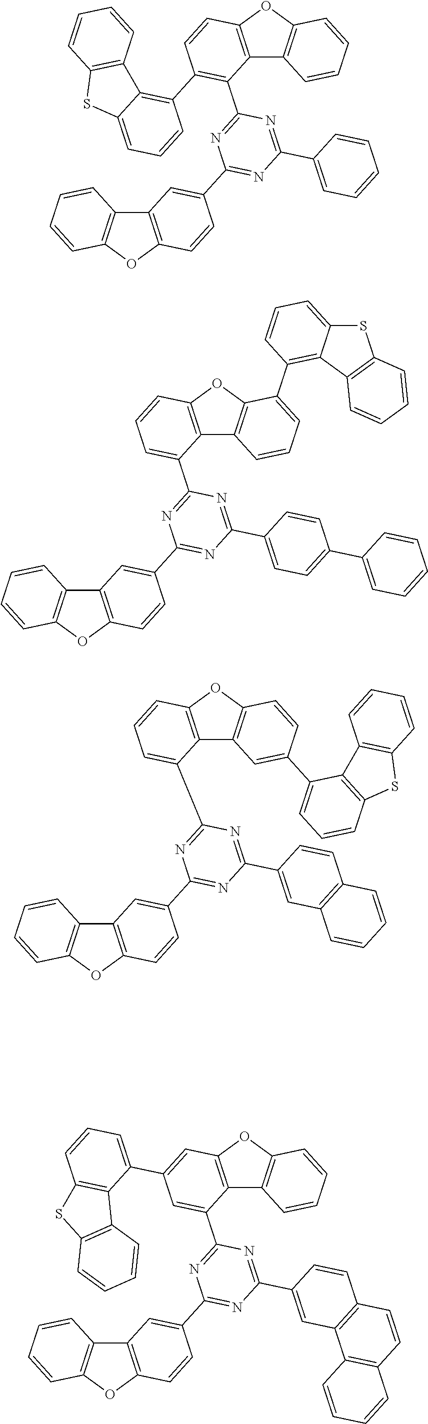 [SMILES: c1ccc(-c2nc(-c3ccc4oc5ccccc5c4c3)nc(-c3c(-c4cccc5sc6ccccc6c45)ccc4oc5ccccc5c34)n2)cc1 ; c1ccc(-c2ccc(-c3nc(-c4ccc5oc6ccccc6c5c4)nc(-c4cccc5oc6c(-c7cccc8sc9ccccc9c78)cccc6c45)n3)cc2)cc1 ; c1ccc2cc(-c3nc(-c4ccc5oc6ccccc6c5c4)nc(-c4cccc5oc6ccc(-c7cccc8sc9ccccc9c78)cc6c45)n3)ccc2c1 ; c1ccc2c(c1)ccc1ccc(-c3nc(-c4ccc5oc6ccccc6c5c4)nc(-c4cc(-c5cccc6sc7ccccc7c56)cc5oc6ccccc6c45)n3)cc12]

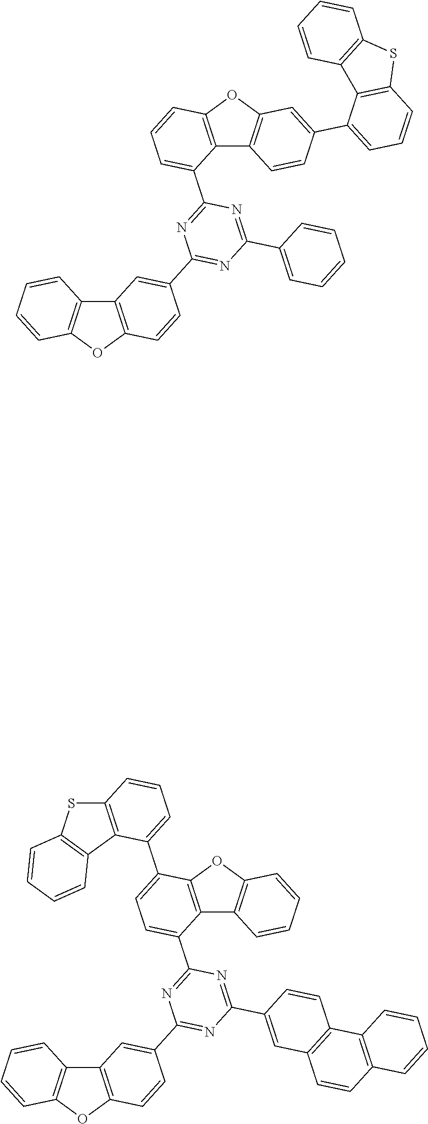 [SMILES: c1ccc(-c2nc(-c3ccc4oc5ccccc5c4c3)nc(-c3cccc4oc5cc(-c6cccc7sc8ccccc8c67)ccc5c34)n2)cc1 ; c1ccc2c(c1)ccc1cc(-c3nc(-c4ccc5oc6ccccc6c5c4)nc(-c4ccc(-c5cccc6sc7ccccc7c56)c5oc6ccccc6c45)n3)ccc12]

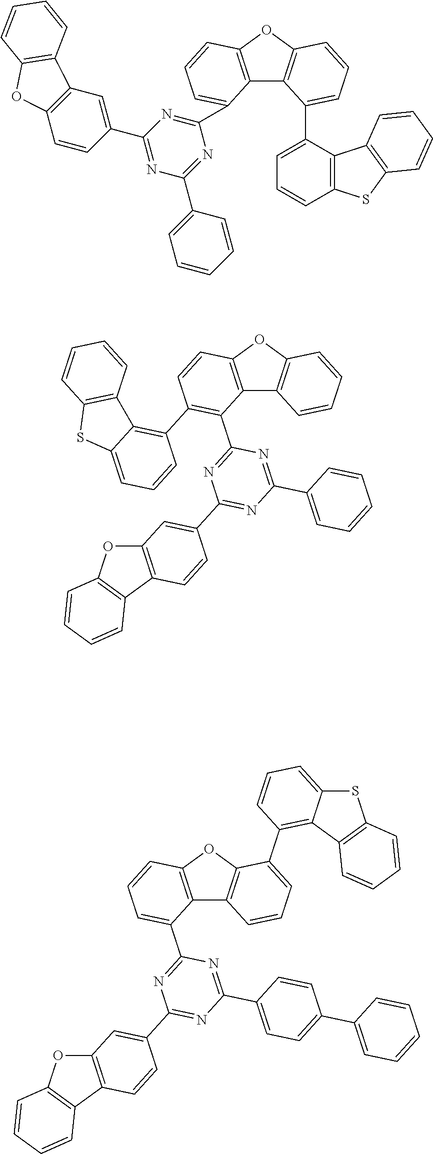 [SMILES: c1ccc(-c2nc(-c3ccc4oc5ccccc5c4c3)nc(-c3cccc4oc5cccc(-c6cccc7sc8ccccc8c67)c5c34)n2)cc1 ; c1ccc(-c2nc(-c3ccc4c(c3)oc3ccccc34)nc(-c3c(-c4cccc5sc6ccccc6c45)ccc4oc5ccccc5c34)n2)cc1 ; c1ccc(-c2ccc(-c3nc(-c4ccc5c(c4)oc4ccccc45)nc(-c4cccc5oc6c(-c7cccc8sc9ccccc9c78)cccc6c45)n3)cc2)cc1]

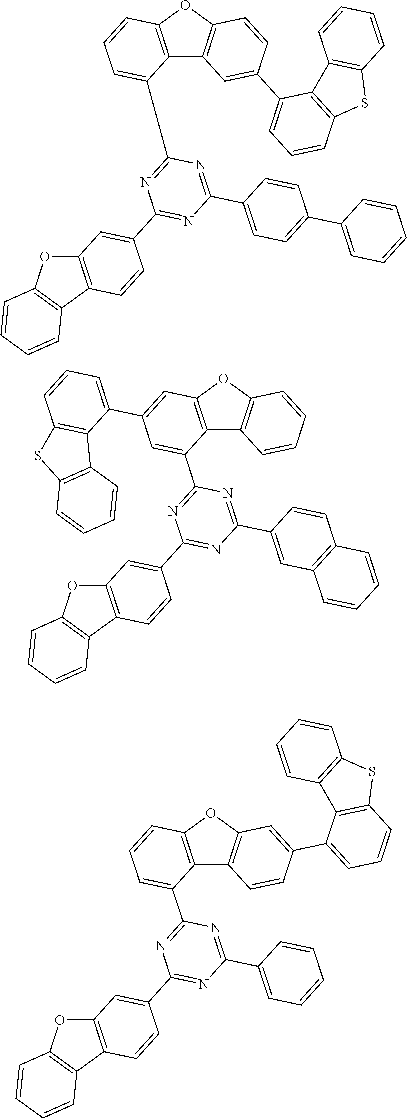

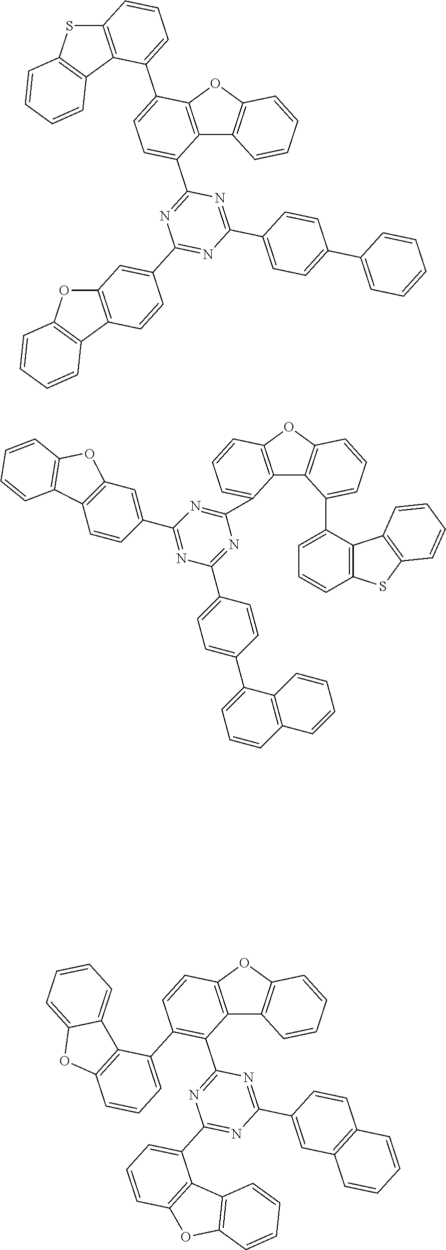 [SMILES: c1ccc(-c2ccc(-c3nc(-c4ccc5c(c4)oc4ccccc45)nc(-c4ccc(-c5cccc6sc7ccccc7c56)c5oc6ccccc6c45)n3)cc2)cc1 ; c1ccc2c(-c3ccc(-c4nc(-c5ccc6c(c5)oc5ccccc56)nc(-c5cccc6oc7cccc(-c8cccc9sc%10ccccc%10c89)c7c56)n4)cc3)cccc2c1 ; c1ccc2cc(-c3nc(-c4cccc5oc6ccccc6c45)nc(-c4c(-c5cccc6oc7ccccc7c56)ccc5oc6ccccc6c45)n3)ccc2c1]

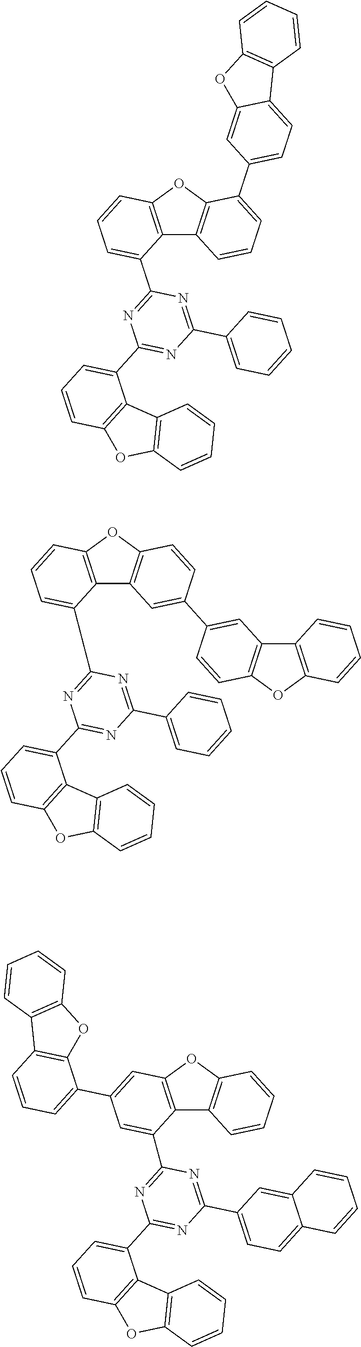 [SMILES: c1ccc(-c2nc(-c3cccc4oc5ccccc5c34)nc(-c3cccc4oc5c(-c6ccc7c(c6)oc6ccccc67)cccc5c34)n2)cc1 ; c1ccc(-c2nc(-c3cccc4oc5ccccc5c34)nc(-c3cccc4oc5ccc(-c6ccc7oc8ccccc8c7c6)cc5c34)n2)cc1 ; c1ccc2cc(-c3nc(-c4cccc5oc6ccccc6c45)nc(-c4cc(-c5cccc6c5oc5ccccc56)cc5oc6ccccc6c45)n3)ccc2c1]

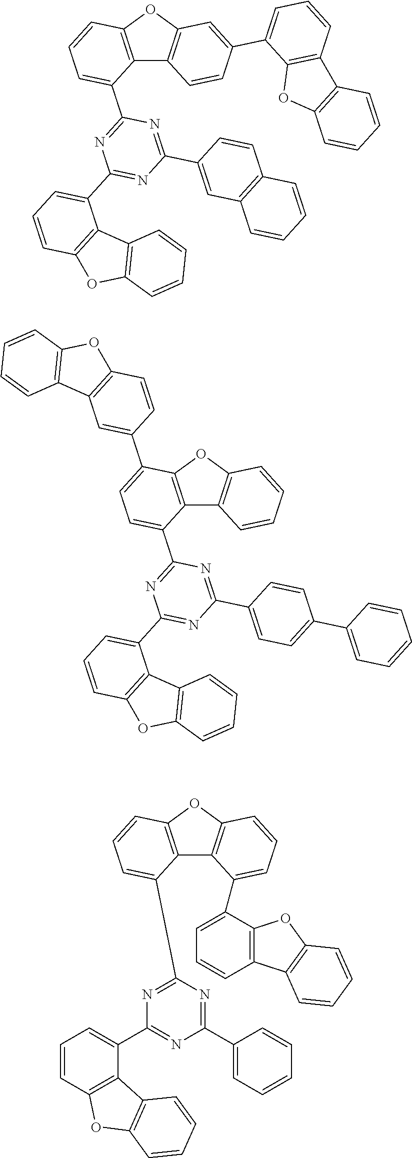 [SMILES: c1ccc2cc(-c3nc(-c4cccc5oc6ccccc6c45)nc(-c4cccc5oc6cc(-c7cccc8c7oc7ccccc78)ccc6c45)n3)ccc2c1 ; c1ccc(-c2ccc(-c3nc(-c4cccc5oc6ccccc6c45)nc(-c4ccc(-c5ccc6oc7ccccc7c6c5)c5oc6ccccc6c45)n3)cc2)cc1 ; c1ccc(-c2nc(-c3cccc4oc5ccccc5c34)nc(-c3cccc4oc5cccc(-c6cccc7c6oc6ccccc67)c5c34)n2)cc1]

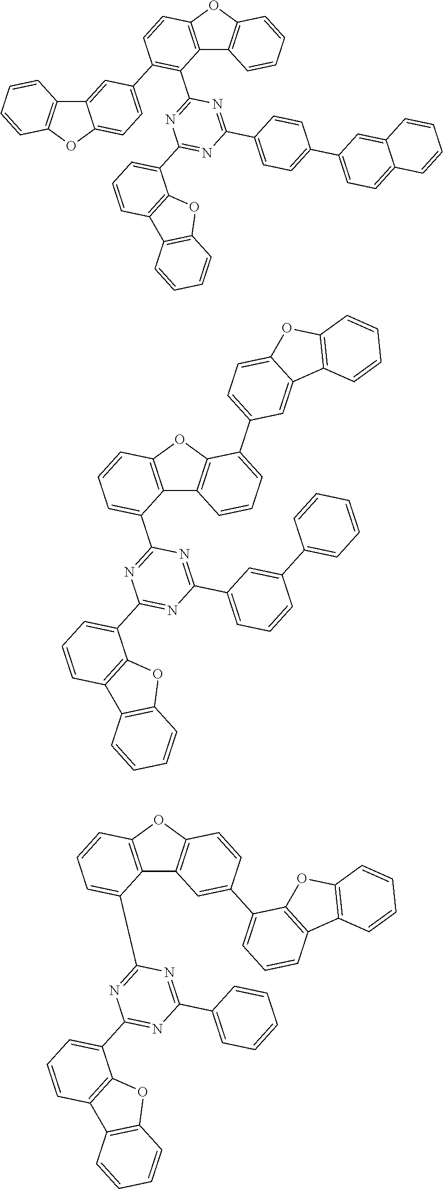 [SMILES: c1ccc2cc(-c3ccc(-c4nc(-c5cccc6c5oc5ccccc56)nc(-c5c(-c6ccc7oc8ccccc8c7c6)ccc6oc7ccccc7c56)n4)cc3)ccc2c1 ; c1ccc(-c2cccc(-c3nc(-c4cccc5c4oc4ccccc45)nc(-c4cccc5oc6c(-c7ccc8oc9ccccc9c8c7)cccc6c45)n3)c2)cc1 ; c1ccc(-c2nc(-c3cccc4c3oc3ccccc34)nc(-c3cccc4oc5ccc(-c6cccc7c6oc6ccccc67)cc5c34)n2)cc1]

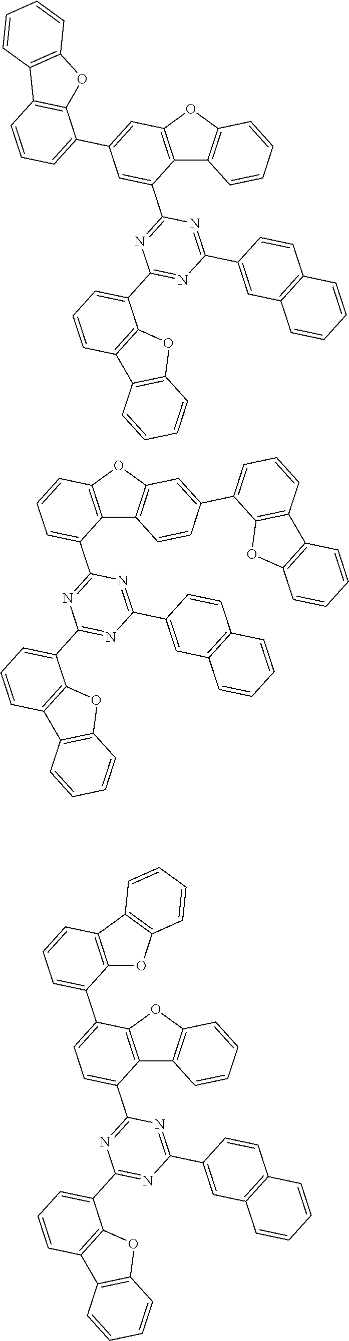

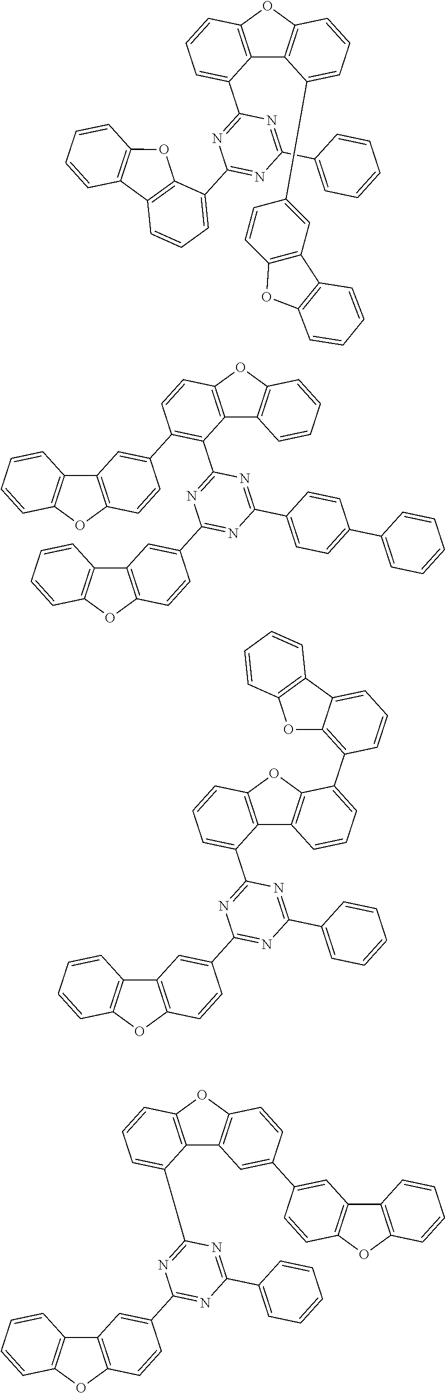

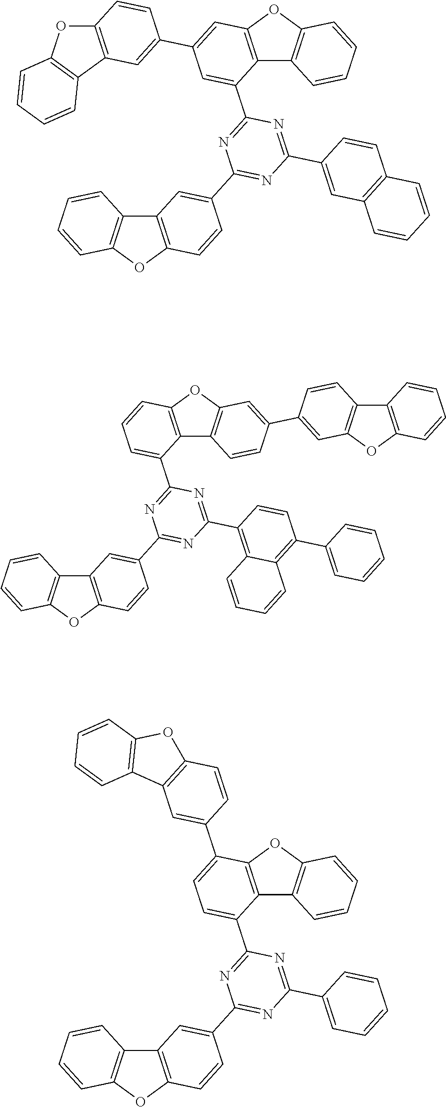 [SMILES: c1ccc2cc(-c3nc(-c4ccc5oc6ccccc6c5c4)nc(-c4cc(-c5ccc6oc7ccccc7c6c5)cc5oc6ccccc6c45)n3)ccc2c1 ; c1ccc(-c2ccc(-c3nc(-c4ccc5oc6ccccc6c5c4)nc(-c4cccc5oc6cc(-c7ccc8c(c7)oc7ccccc78)ccc6c45)n3)c3ccccc23)cc1 ; c1ccc(-c2nc(-c3ccc4oc5ccccc5c4c3)nc(-c3ccc(-c4ccc5oc6ccccc6c5c4)c4oc5ccccc5c34)n2)cc1]

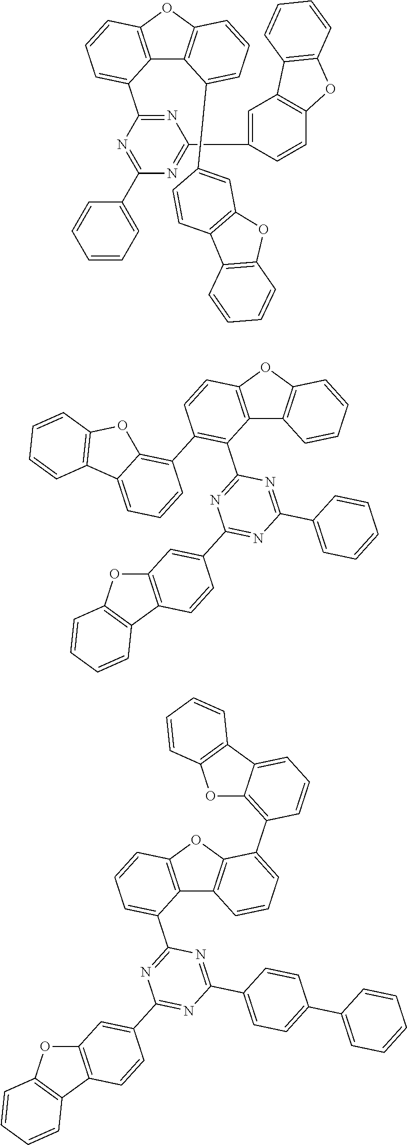 [SMILES: c1ccc(-c2nc(-c3ccc4oc5ccccc5c4c3)nc(-c3cccc4oc5cccc(-c6ccc7c(c6)oc6ccccc67)c5c34)n2)cc1 ; c1ccc(-c2nc(-c3ccc4c(c3)oc3ccccc34)nc(-c3c(-c4cccc5c4oc4ccccc45)ccc4oc5ccccc5c34)n2)cc1 ; c1ccc(-c2ccc(-c3nc(-c4ccc5c(c4)oc4ccccc45)nc(-c4cccc5oc6c(-c7cccc8c7oc7ccccc78)cccc6c45)n3)cc2)cc1]

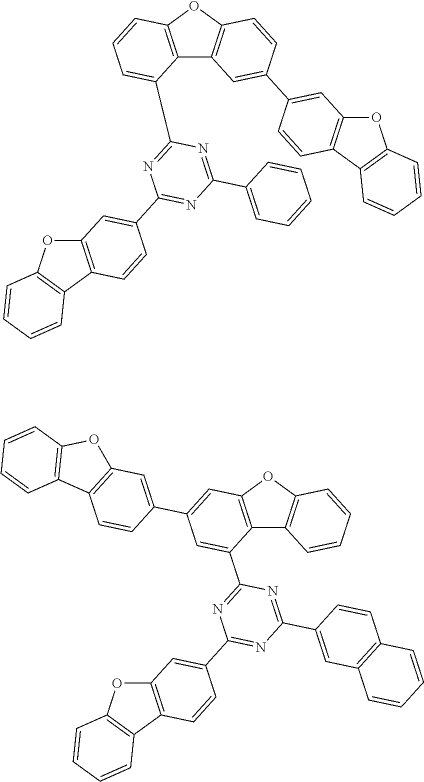 [SMILES: c1ccc(-c2nc(-c3ccc4c(c3)oc3ccccc34)nc(-c3cccc4oc5ccc(-c6ccc7c(c6)oc6ccccc67)cc5c34)n2)cc1 ; c1ccc2cc(-c3nc(-c4ccc5c(c4)oc4ccccc45)nc(-c4cc(-c5ccc6c(c5)oc5ccccc56)cc5oc6ccccc6c45)n3)ccc2c1]

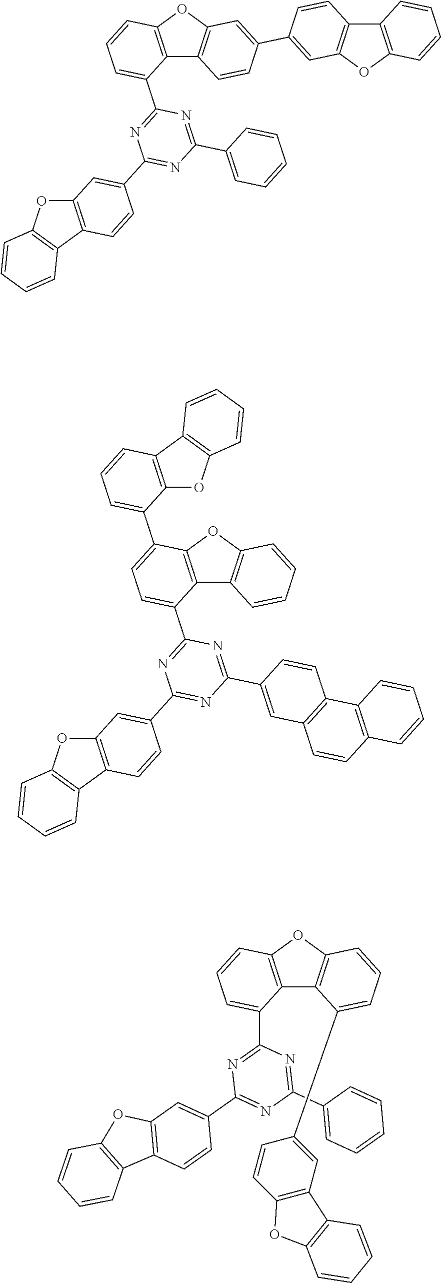 [SMILES: c1ccc(-c2nc(-c3ccc4c(c3)oc3ccccc34)nc(-c3cccc4oc5cc(-c6ccc7c(c6)oc6ccccc67)ccc5c34)n2)cc1 ; c1ccc2c(c1)ccc1cc(-c3nc(-c4ccc5c(c4)oc4ccccc45)nc(-c4ccc(-c5cccc6c5oc5ccccc56)c5oc6ccccc6c45)n3)ccc12 ; c1ccc(-c2nc(-c3ccc4c(c3)oc3ccccc34)nc(-c3cccc4oc5cccc(-c6ccc7oc8ccccc8c7c6)c5c34)n2)cc1]

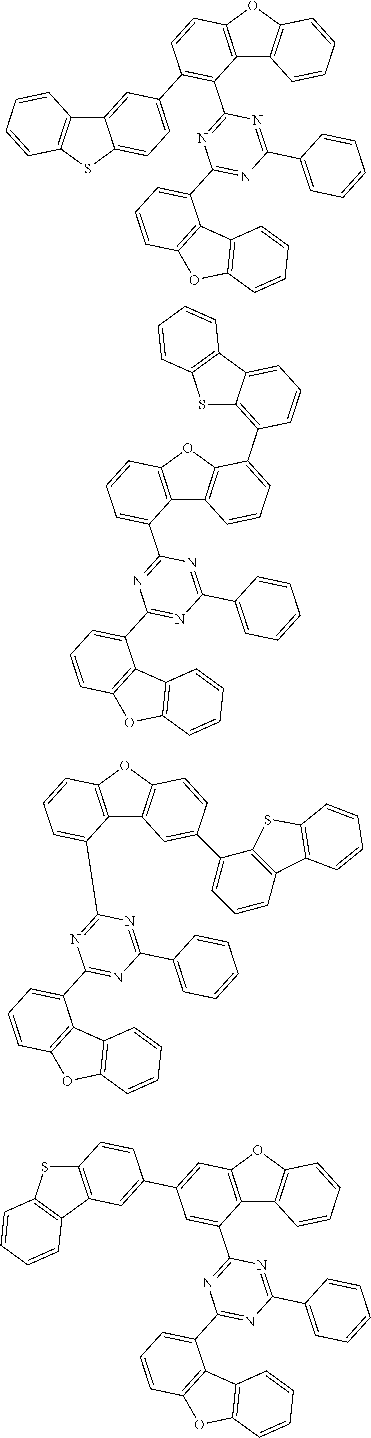

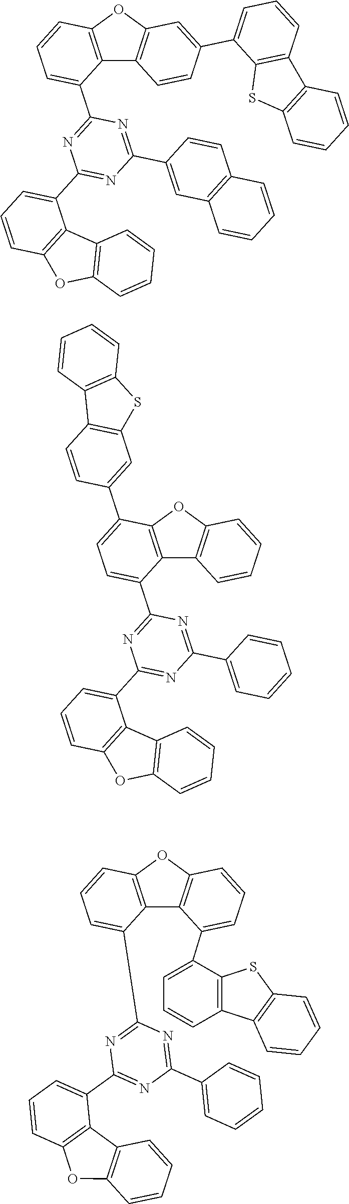

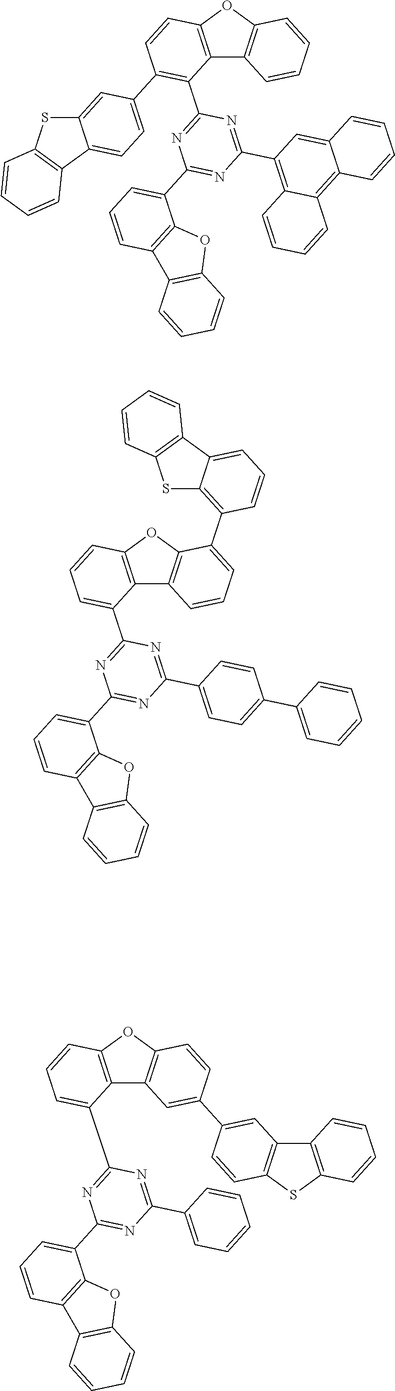 [SMILES: c1ccc2c(c1)cc(-c1nc(-c3cccc4c3oc3ccccc34)nc(-c3c(-c4ccc5c(c4)sc4ccccc45)ccc4oc5ccccc5c34)n1)c1ccccc12 ; c1ccc(-c2ccc(-c3nc(-c4cccc5c4oc4ccccc45)nc(-c4cccc5oc6c(-c7cccc8c7sc7ccccc78)cccc6c45)n3)cc2)cc1 ; c1ccc(-c2nc(-c3cccc4c3oc3ccccc34)nc(-c3cccc4oc5ccc(-c6ccc7sc8ccccc8c7c6)cc5c34)n2)cc1]

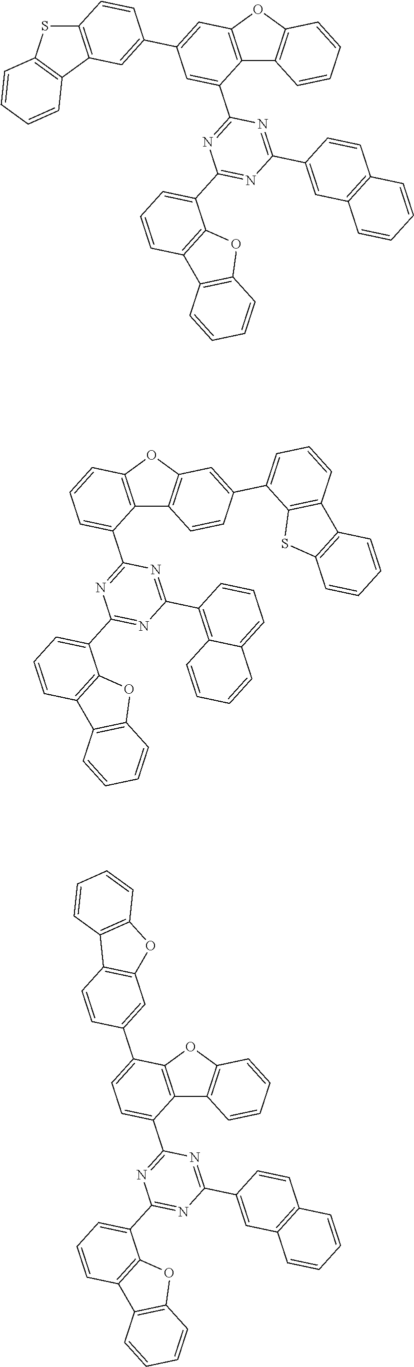 [SMILES: c1ccc2cc(-c3nc(-c4cccc5c4oc4ccccc45)nc(-c4cc(-c5ccc6sc7ccccc7c6c5)cc5oc6ccccc6c45)n3)ccc2c1 ; c1ccc2c(-c3nc(-c4cccc5c4oc4ccccc45)nc(-c4cccc5oc6cc(-c7cccc8c7sc7ccccc78)ccc6c45)n3)cccc2c1 ; c1ccc2cc(-c3nc(-c4cccc5c4oc4ccccc45)nc(-c4ccc(-c5ccc6c(c5)oc5ccccc56)c5oc6ccccc6c45)n3)ccc2c1]

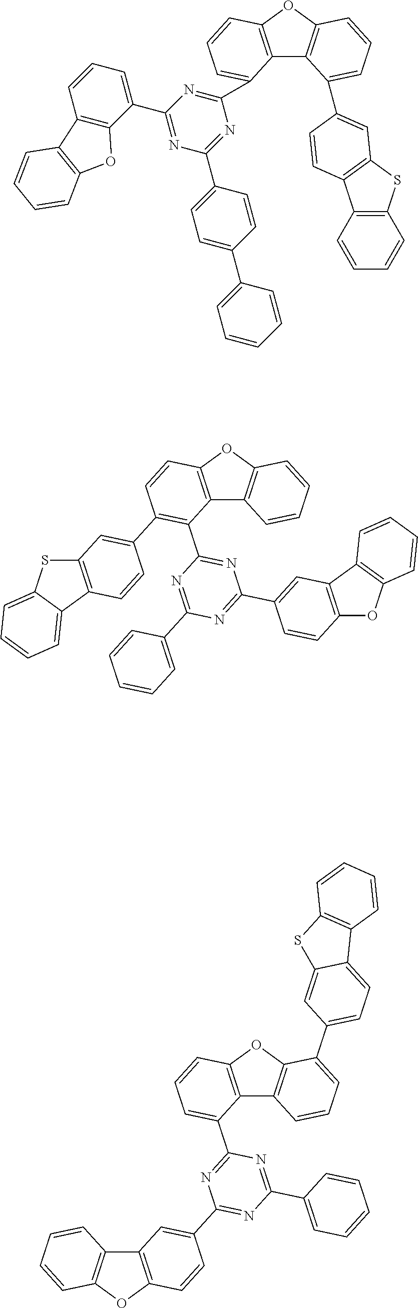 [SMILES: c1ccc(-c2ccc(-c3nc(-c4cccc5c4oc4ccccc45)nc(-c4cccc5oc6cccc(-c7ccc8c(c7)sc7ccccc78)c6c45)n3)cc2)cc1 ; c1ccc(-c2nc(-c3ccc4oc5ccccc5c4c3)nc(-c3c(-c4ccc5c(c4)sc4ccccc45)ccc4oc5ccccc5c34)n2)cc1 ; c1ccc(-c2nc(-c3ccc4oc5ccccc5c4c3)nc(-c3cccc4oc5c(-c6ccc7c(c6)sc6ccccc67)cccc5c34)n2)cc1]

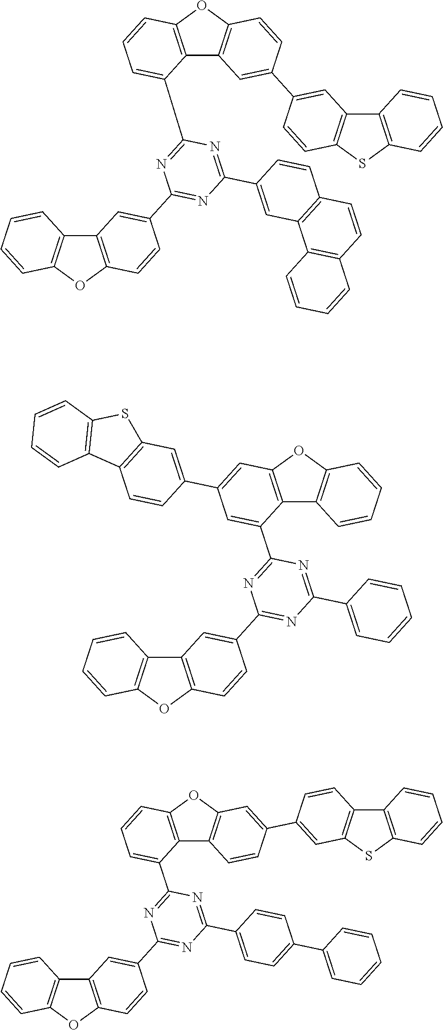 [SMILES: c1ccc2c(c1)ccc1ccc(-c3nc(-c4ccc5oc6ccccc6c5c4)nc(-c4cccc5oc6ccc(-c7ccc8sc9ccccc9c8c7)cc6c45)n3)cc12 ; c1ccc(-c2nc(-c3ccc4oc5ccccc5c4c3)nc(-c3cc(-c4ccc5c(c4)sc4ccccc45)cc4oc5ccccc5c34)n2)cc1 ; c1ccc(-c2ccc(-c3nc(-c4ccc5oc6ccccc6c5c4)nc(-c4cccc5oc6cc(-c7ccc8c(c7)sc7ccccc78)ccc6c45)n3)cc2)cc1]

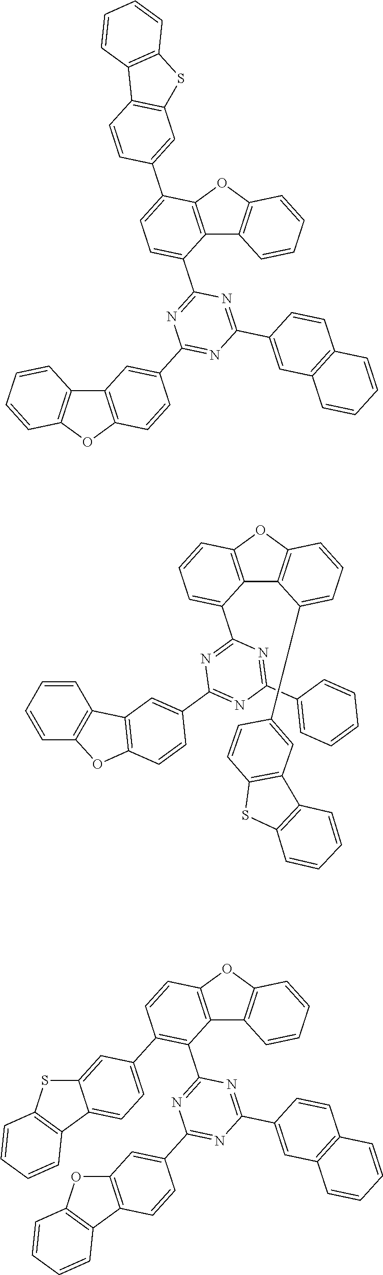 [SMILES: c1ccc2cc(-c3nc(-c4ccc5oc6ccccc6c5c4)nc(-c4ccc(-c5ccc6c(c5)sc5ccccc56)c5oc6ccccc6c45)n3)ccc2c1 ; c1ccc(-c2nc(-c3ccc4oc5ccccc5c4c3)nc(-c3cccc4oc5cccc(-c6ccc7sc8ccccc8c7c6)c5c34)n2)cc1 ; c1ccc2cc(-c3nc(-c4ccc5c(c4)oc4ccccc45)nc(-c4c(-c5ccc6c(c5)sc5ccccc56)ccc5oc6ccccc6c45)n3)ccc2c1]

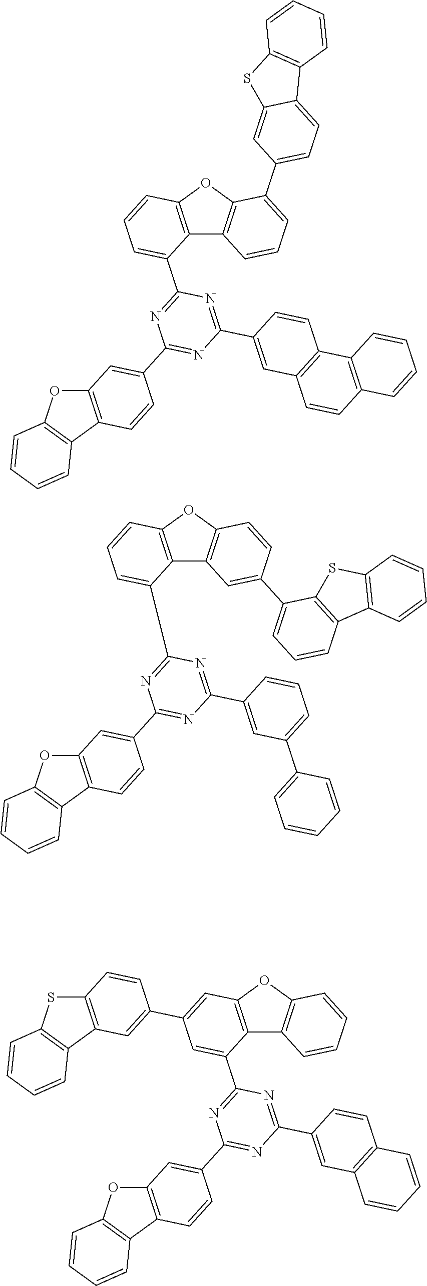 [SMILES: c1ccc2c(c1)ccc1cc(-c3nc(-c4ccc5c(c4)oc4ccccc45)nc(-c4cccc5oc6c(-c7ccc8c(c7)sc7ccccc78)cccc6c45)n3)ccc12 ; c1ccc(-c2cccc(-c3nc(-c4ccc5c(c4)oc4ccccc45)nc(-c4cccc5oc6ccc(-c7cccc8c7sc7ccccc78)cc6c45)n3)c2)cc1 ; c1ccc2cc(-c3nc(-c4ccc5c(c4)oc4ccccc45)nc(-c4cc(-c5ccc6sc7ccccc7c6c5)cc5oc6ccccc6c45)n3)ccc2c1]

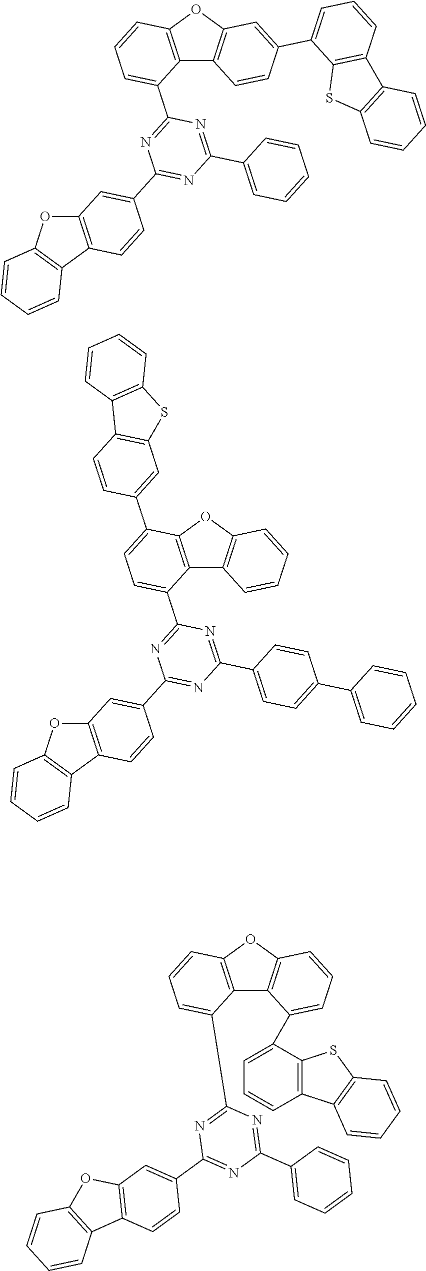 [SMILES: c1ccc(-c2nc(-c3ccc4c(c3)oc3ccccc34)nc(-c3cccc4oc5cc(-c6cccc7c6sc6ccccc67)ccc5c34)n2)cc1 ; c1ccc(-c2ccc(-c3nc(-c4ccc5c(c4)oc4ccccc45)nc(-c4ccc(-c5ccc6c(c5)sc5ccccc56)c5oc6ccccc6c45)n3)cc2)cc1 ; c1ccc(-c2nc(-c3ccc4c(c3)oc3ccccc34)nc(-c3cccc4oc5cccc(-c6cccc7c6sc6ccccc67)c5c34)n2)cc1]

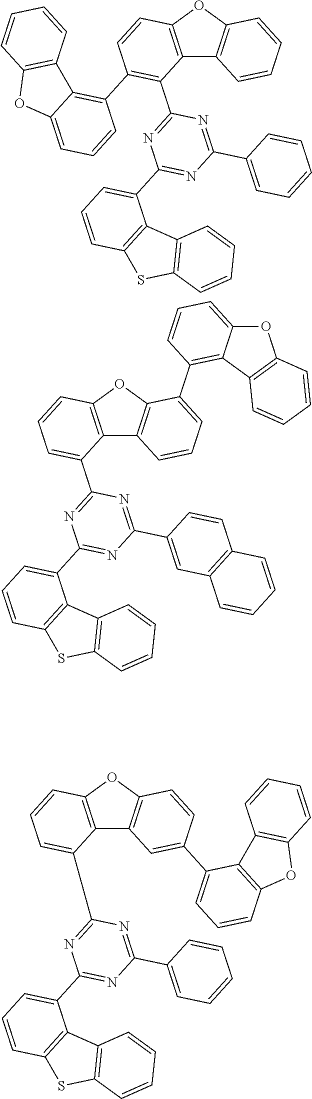

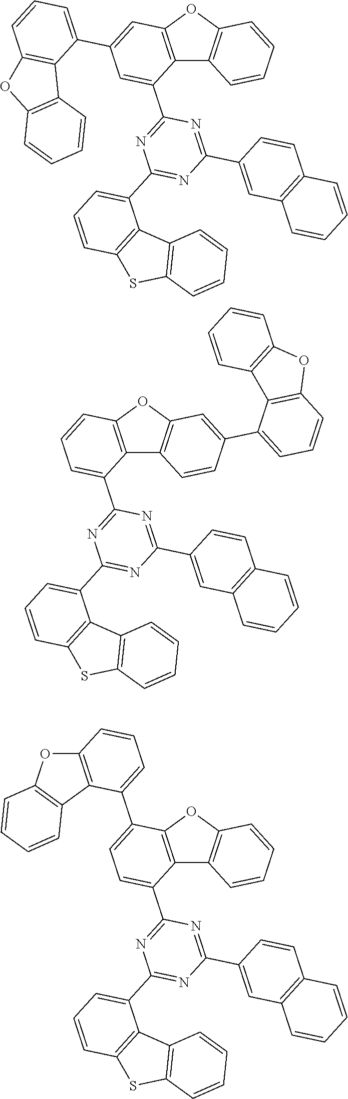

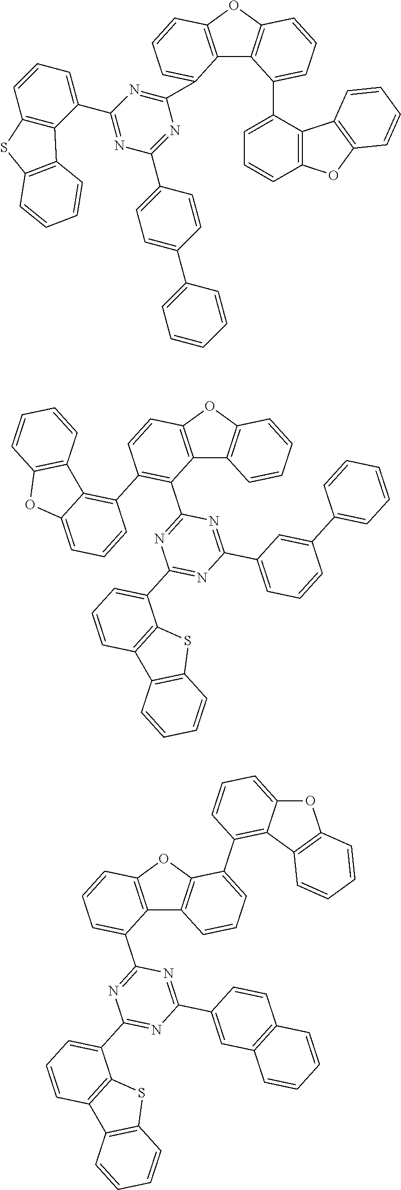 [SMILES: c1ccc(-c2ccc(-c3nc(-c4cccc5sc6ccccc6c45)nc(-c4cccc5oc6cccc(-c7cccc8oc9ccccc9c78)c6c45)n3)cc2)cc1 ; c1ccc(-c2cccc(-c3nc(-c4cccc5c4sc4ccccc45)nc(-c4c(-c5cccc6oc7ccccc7c56)ccc5oc6ccccc6c45)n3)c2)cc1 ; c1ccc2cc(-c3nc(-c4cccc5c4sc4ccccc45)nc(-c4cccc5oc6c(-c7cccc8oc9ccccc9c78)cccc6c45)n3)ccc2c1]

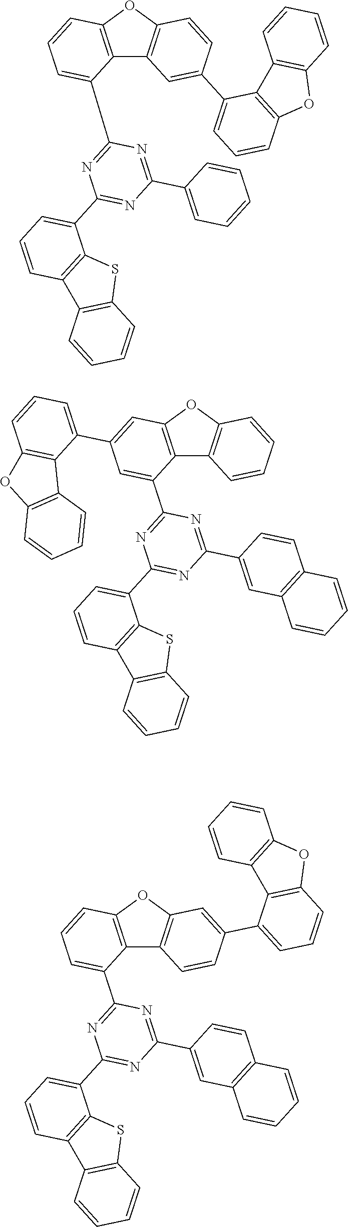

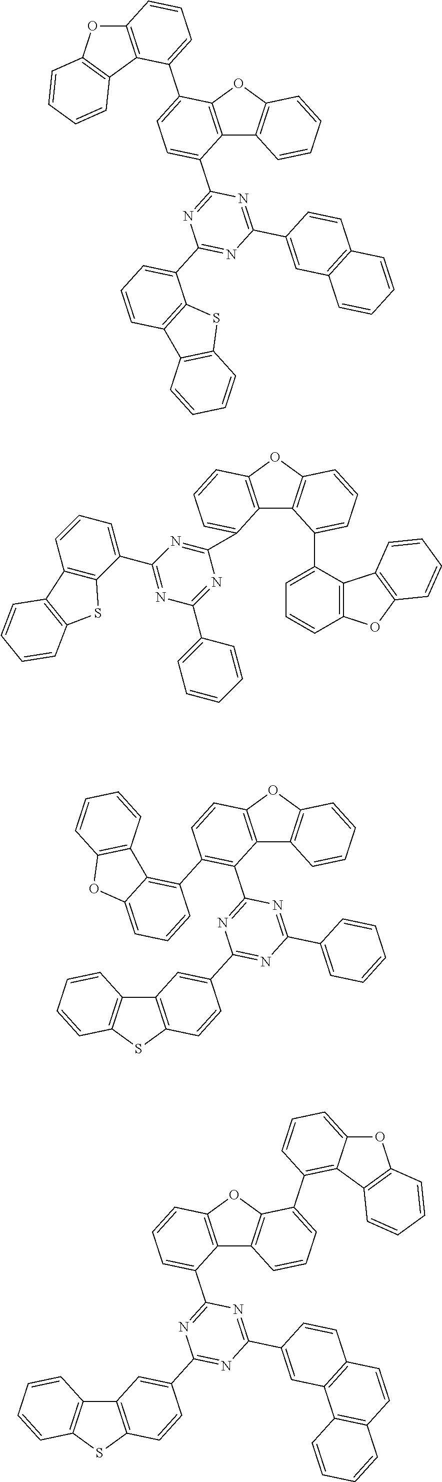 [SMILES: c1ccc2cc(-c3nc(-c4cccc5c4sc4ccccc45)nc(-c4ccc(-c5cccc6oc7ccccc7c56)c5oc6ccccc6c45)n3)ccc2c1 ; c1ccc(-c2nc(-c3cccc4c3sc3ccccc34)nc(-c3cccc4oc5cccc(-c6cccc7oc8ccccc8c67)c5c34)n2)cc1 ; c1ccc(-c2nc(-c3ccc4sc5ccccc5c4c3)nc(-c3c(-c4cccc5oc6ccccc6c45)ccc4oc5ccccc5c34)n2)cc1 ; c1ccc2c(c1)ccc1ccc(-c3nc(-c4ccc5sc6ccccc6c5c4)nc(-c4cccc5oc6c(-c7cccc8oc9ccccc9c78)cccc6c45)n3)cc12]

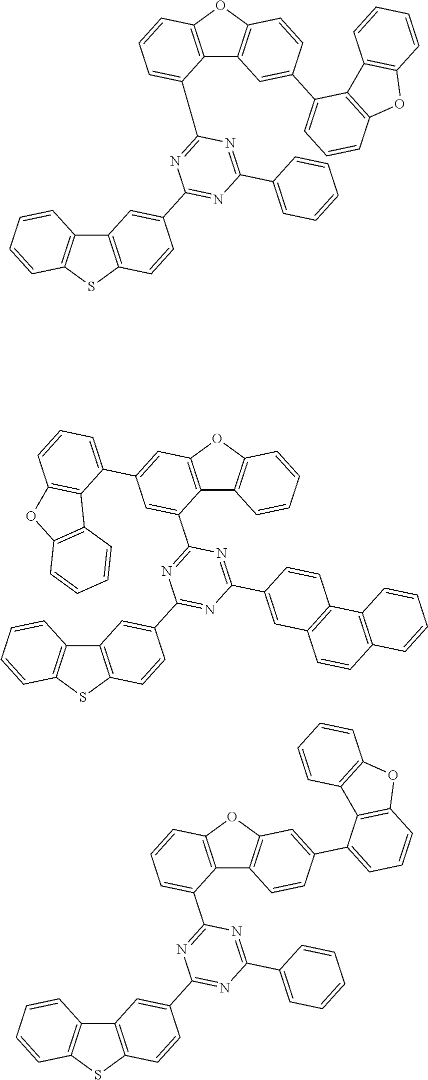 [SMILES: c1ccc(-c2nc(-c3ccc4sc5ccccc5c4c3)nc(-c3cccc4oc5ccc(-c6cccc7oc8ccccc8c67)cc5c34)n2)cc1 ; c1ccc2c(c1)ccc1cc(-c3nc(-c4ccc5sc6ccccc6c5c4)nc(-c4cc(-c5cccc6oc7ccccc7c56)cc5oc6ccccc6c45)n3)ccc12 ; c1ccc(-c2nc(-c3ccc4sc5ccccc5c4c3)nc(-c3cccc4oc5cc(-c6cccc7oc8ccccc8c67)ccc5c34)n2)cc1]

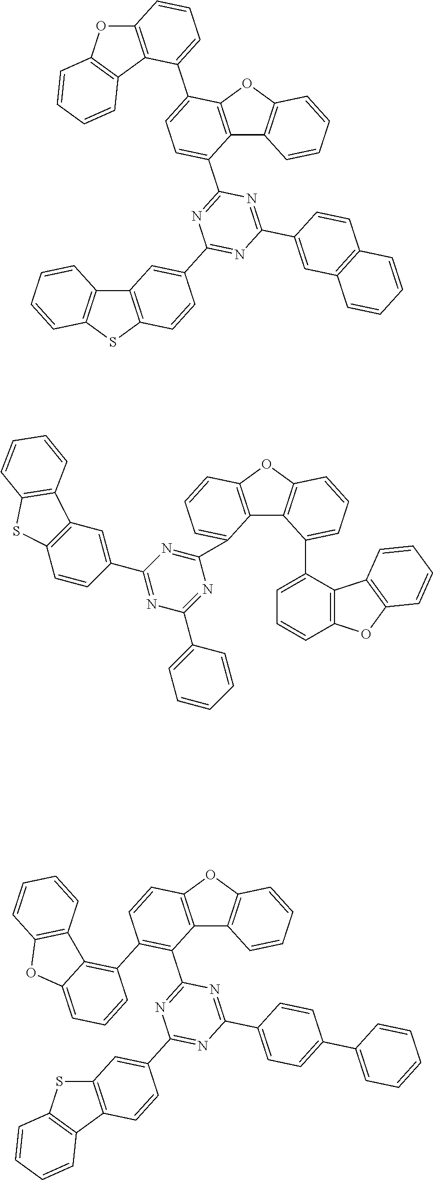 [SMILES: c1ccc2cc(-c3nc(-c4ccc5sc6ccccc6c5c4)nc(-c4ccc(-c5cccc6oc7ccccc7c56)c5oc6ccccc6c45)n3)ccc2c1 ; c1ccc(-c2nc(-c3ccc4sc5ccccc5c4c3)nc(-c3cccc4oc5cccc(-c6cccc7oc8ccccc8c67)c5c34)n2)cc1 ; c1ccc(-c2ccc(-c3nc(-c4ccc5c(c4)sc4ccccc45)nc(-c4c(-c5cccc6oc7ccccc7c56)ccc5oc6ccccc6c45)n3)cc2)cc1]

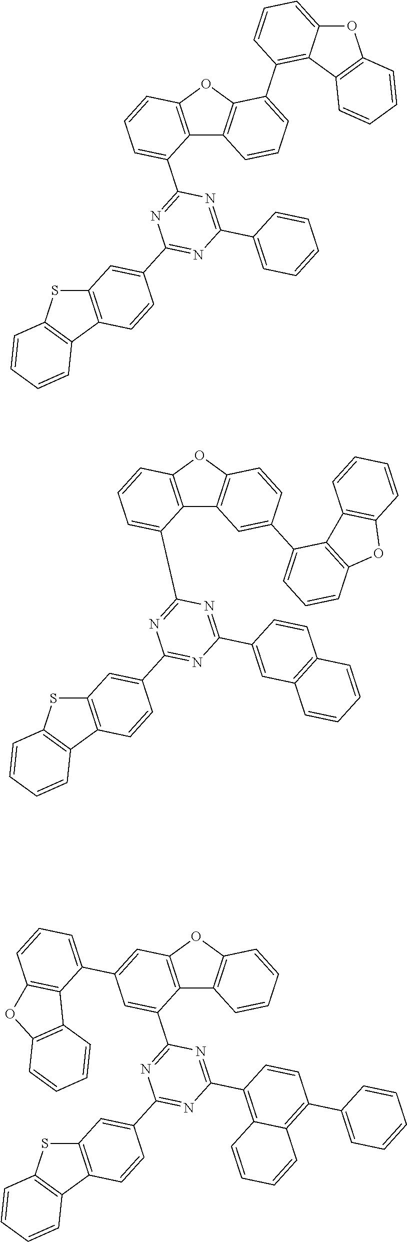 [SMILES: c1ccc(-c2nc(-c3ccc4c(c3)sc3ccccc34)nc(-c3cccc4oc5c(-c6cccc7oc8ccccc8c67)cccc5c34)n2)cc1 ; c1ccc2cc(-c3nc(-c4ccc5c(c4)sc4ccccc45)nc(-c4cccc5oc6ccc(-c7cccc8oc9ccccc9c78)cc6c45)n3)ccc2c1 ; c1ccc(-c2ccc(-c3nc(-c4ccc5c(c4)sc4ccccc45)nc(-c4cc(-c5cccc6oc7ccccc7c56)cc5oc6ccccc6c45)n3)c3ccccc23)cc1]

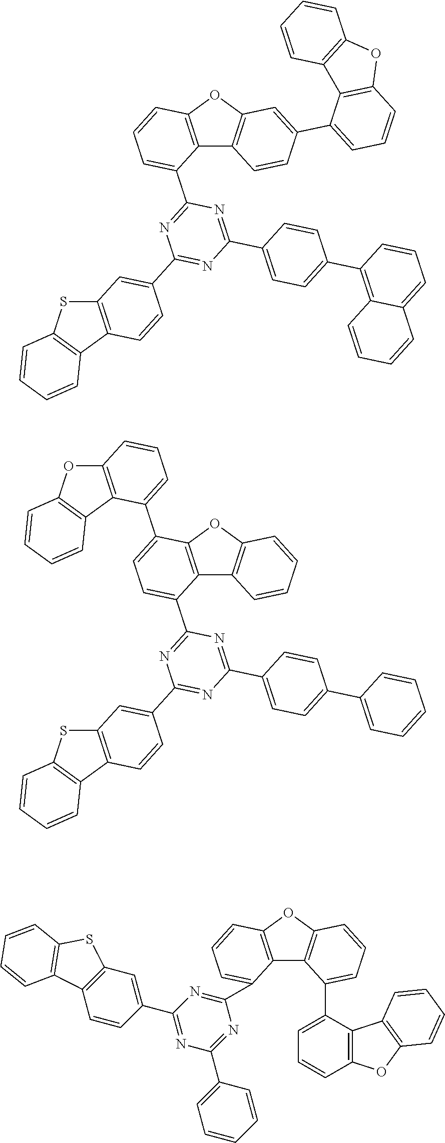 [SMILES: c1ccc2c(-c3ccc(-c4nc(-c5ccc6c(c5)sc5ccccc56)nc(-c5cccc6oc7cc(-c8cccc9oc%10ccccc%10c89)ccc7c56)n4)cc3)cccc2c1 ; c1ccc(-c2ccc(-c3nc(-c4ccc5c(c4)sc4ccccc45)nc(-c4ccc(-c5cccc6oc7ccccc7c56)c5oc6ccccc6c45)n3)cc2)cc1 ; c1ccc(-c2nc(-c3ccc4c(c3)sc3ccccc34)nc(-c3cccc4oc5cccc(-c6cccc7oc8ccccc8c67)c5c34)n2)cc1]

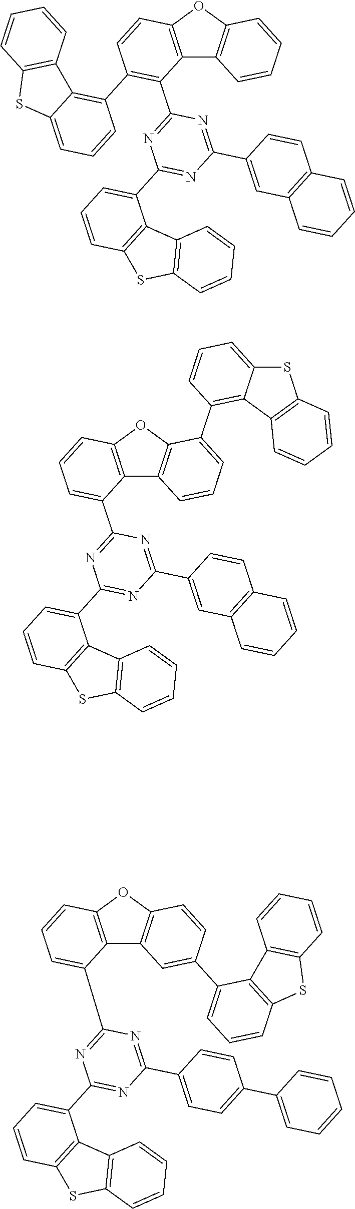 [SMILES: c1ccc2cc(-c3nc(-c4c(-c5cccc6sc7ccccc7c56)ccc5oc6ccccc6c45)nc(-c4cccc5sc6ccccc6c45)n3)ccc2c1 ; c1ccc2cc(-c3nc(-c4cccc5oc6c(-c7cccc8sc9ccccc9c78)cccc6c45)nc(-c4cccc5sc6ccccc6c45)n3)ccc2c1 ; c1ccc(-c2ccc(-c3nc(-c4cccc5oc6ccc(-c7cccc8sc9ccccc9c78)cc6c45)nc(-c4cccc5sc6ccccc6c45)n3)cc2)cc1]

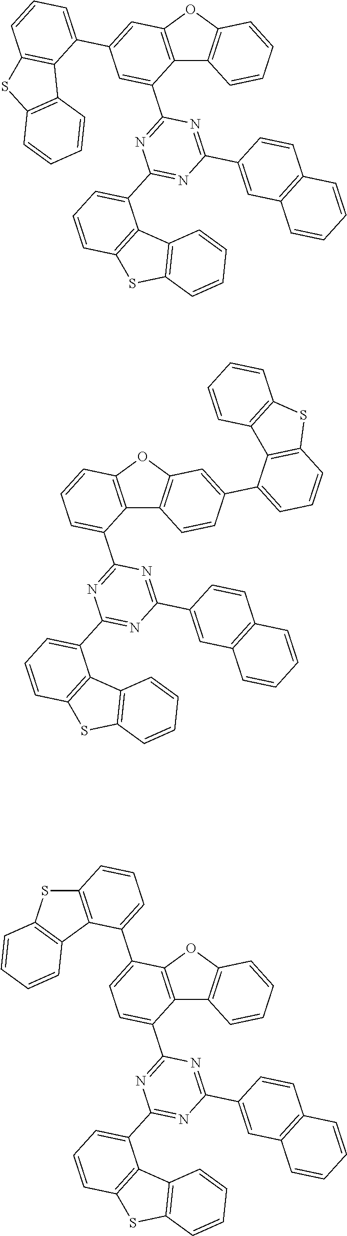 [SMILES: c1ccc2cc(-c3nc(-c4cc(-c5cccc6sc7ccccc7c56)cc5oc6ccccc6c45)nc(-c4cccc5sc6ccccc6c45)n3)ccc2c1 ; c1ccc2cc(-c3nc(-c4cccc5oc6cc(-c7cccc8sc9ccccc9c78)ccc6c45)nc(-c4cccc5sc6ccccc6c45)n3)ccc2c1 ; c1ccc2cc(-c3nc(-c4cccc5sc6ccccc6c45)nc(-c4ccc(-c5cccc6sc7ccccc7c56)c5oc6ccccc6c45)n3)ccc2c1]

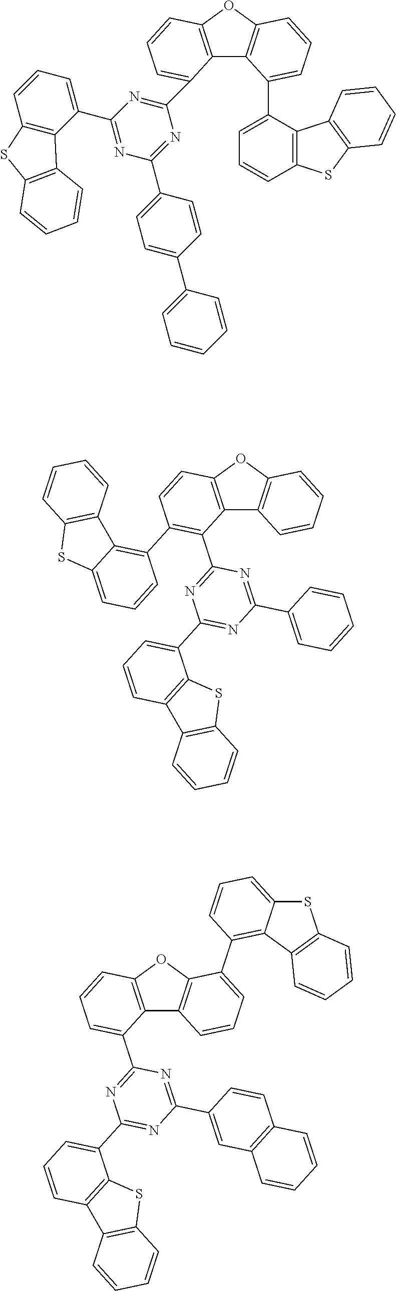 [SMILES: c1ccc(-c2ccc(-c3nc(-c4cccc5sc6ccccc6c45)nc(-c4cccc5oc6cccc(-c7cccc8sc9ccccc9c78)c6c45)n3)cc2)cc1 ; c1ccc(-c2nc(-c3cccc4c3sc3ccccc34)nc(-c3c(-c4cccc5sc6ccccc6c45)ccc4oc5ccccc5c34)n2)cc1 ; c1ccc2cc(-c3nc(-c4cccc5c4sc4ccccc45)nc(-c4cccc5oc6c(-c7cccc8sc9ccccc9c78)cccc6c45)n3)ccc2c1]

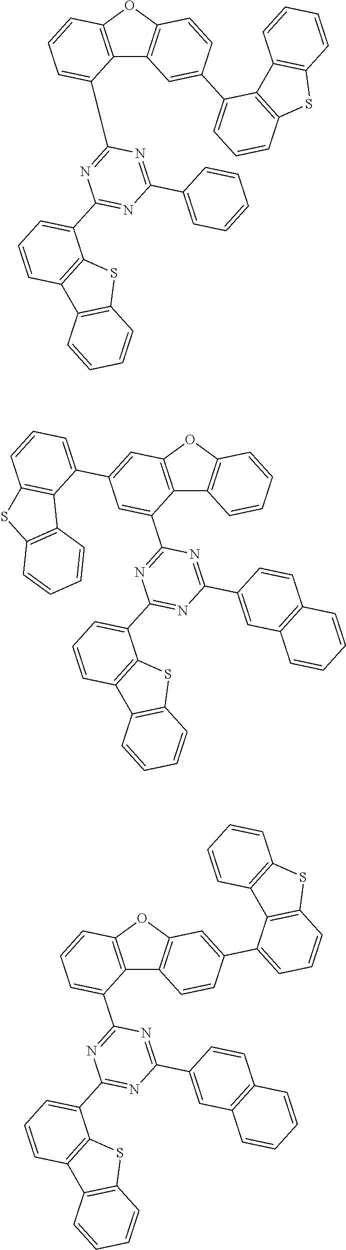 [SMILES: c1ccc(-c2nc(-c3cccc4c3sc3ccccc34)nc(-c3cccc4oc5ccc(-c6cccc7sc8ccccc8c67)cc5c34)n2)cc1 ; c1ccc2cc(-c3nc(-c4cccc5c4sc4ccccc45)nc(-c4cc(-c5cccc6sc7ccccc7c56)cc5oc6ccccc6c45)n3)ccc2c1 ; c1ccc2cc(-c3nc(-c4cccc5c4sc4ccccc45)nc(-c4cccc5oc6cc(-c7cccc8sc9ccccc9c78)ccc6c45)n3)ccc2c1]

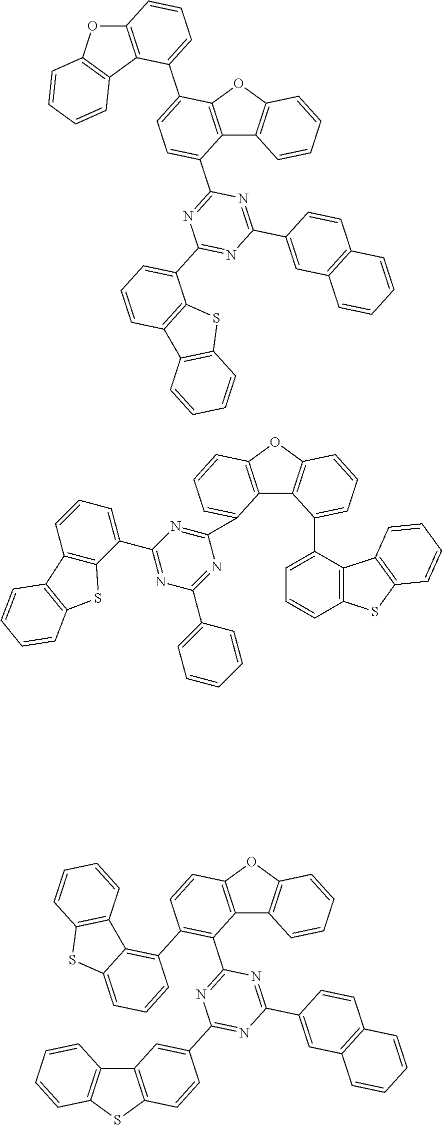

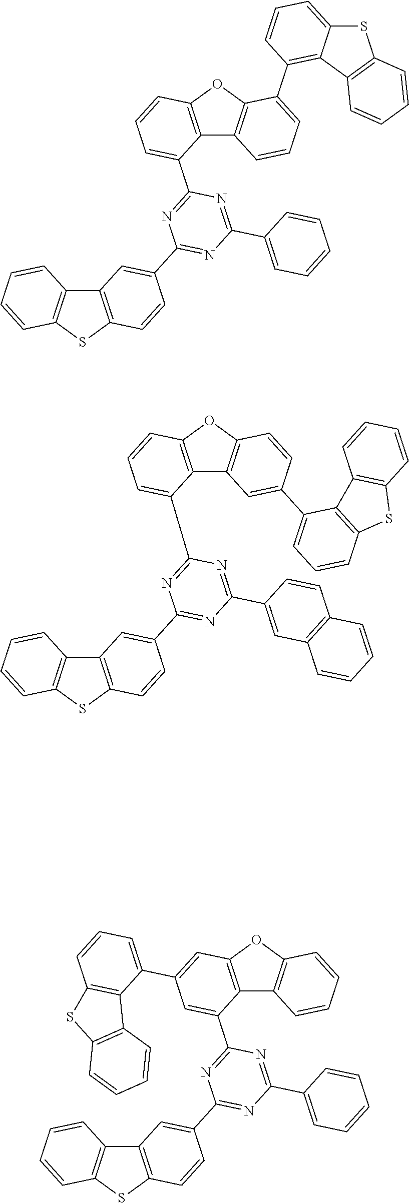 [SMILES: c1ccc(-c2nc(-c3ccc4sc5ccccc5c4c3)nc(-c3cccc4oc5c(-c6cccc7sc8ccccc8c67)cccc5c34)n2)cc1 ; c1ccc2cc(-c3nc(-c4ccc5sc6ccccc6c5c4)nc(-c4cccc5oc6ccc(-c7cccc8sc9ccccc9c78)cc6c45)n3)ccc2c1 ; c1ccc(-c2nc(-c3ccc4sc5ccccc5c4c3)nc(-c3cc(-c4cccc5sc6ccccc6c45)cc4oc5ccccc5c34)n2)cc1]

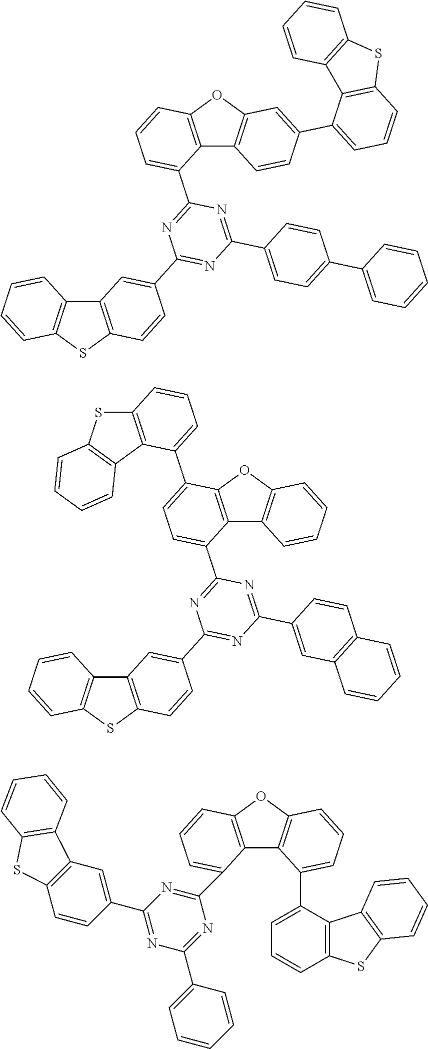 [SMILES: c1ccc(-c2ccc(-c3nc(-c4ccc5sc6ccccc6c5c4)nc(-c4cccc5oc6cc(-c7cccc8sc9ccccc9c78)ccc6c45)n3)cc2)cc1 ; c1ccc2cc(-c3nc(-c4ccc5sc6ccccc6c5c4)nc(-c4ccc(-c5cccc6sc7ccccc7c56)c5oc6ccccc6c45)n3)ccc2c1 ; c1ccc(-c2nc(-c3ccc4sc5ccccc5c4c3)nc(-c3cccc4oc5cccc(-c6cccc7sc8ccccc8c67)c5c34)n2)cc1]

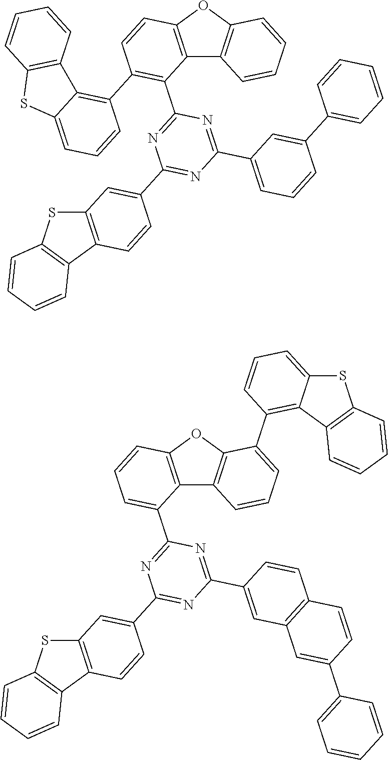 [SMILES: c1ccc(-c2cccc(-c3nc(-c4ccc5c(c4)sc4ccccc45)nc(-c4c(-c5cccc6sc7ccccc7c56)ccc5oc6ccccc6c45)n3)c2)cc1 ; c1ccc(-c2ccc3ccc(-c4nc(-c5ccc6c(c5)sc5ccccc56)nc(-c5cccc6oc7c(-c8cccc9sc%10ccccc%10c89)cccc7c56)n4)cc3c2)cc1]

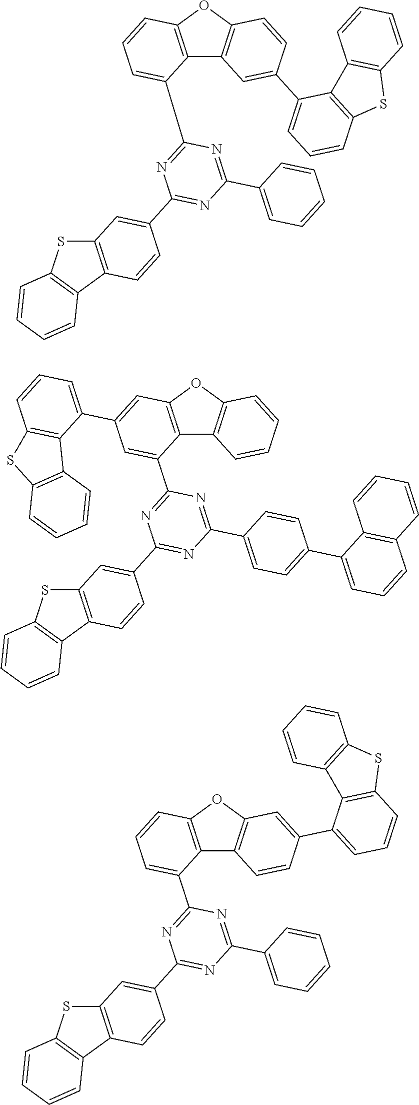

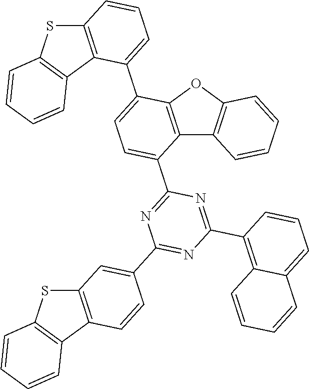 [SMILES: c1ccc2c(-c3nc(-c4ccc5c(c4)sc4ccccc45)nc(-c4ccc(-c5cccc6sc7ccccc7c56)c5oc6ccccc6c45)n3)cccc2c1]

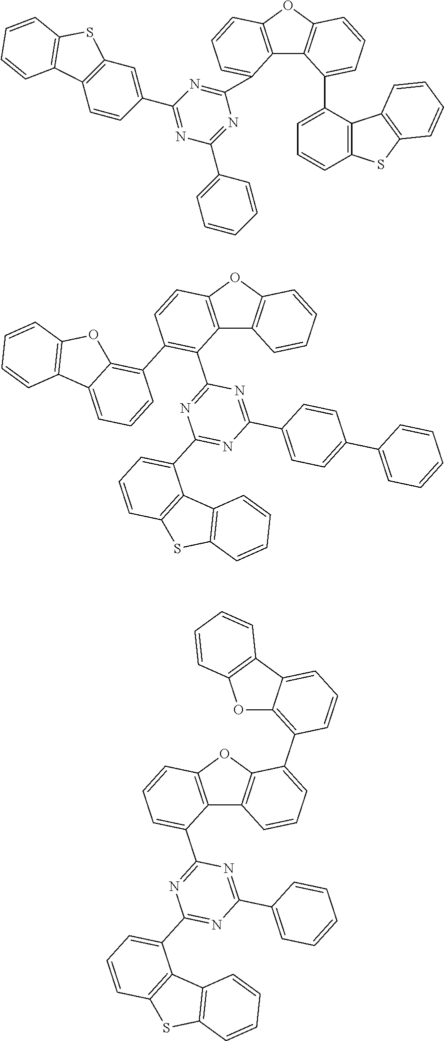

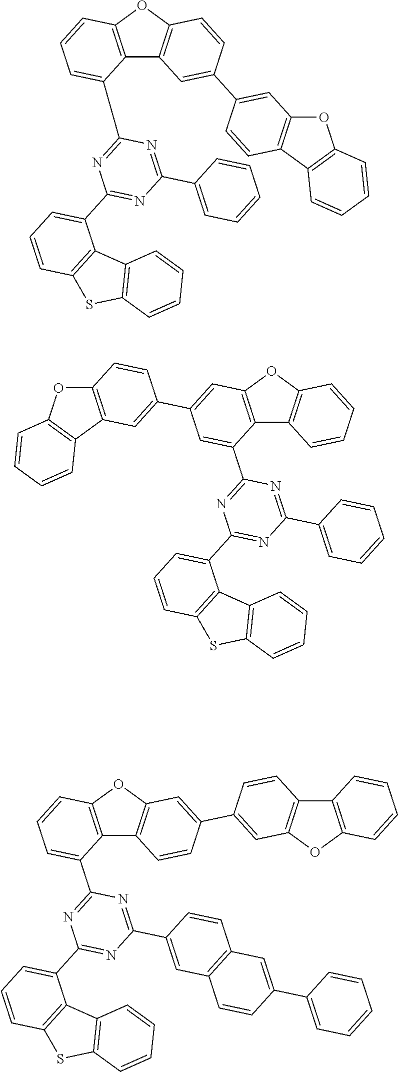 [SMILES: c1ccc(-c2nc(-c3cccc4oc5ccc(-c6ccc7c(c6)oc6ccccc67)cc5c34)nc(-c3cccc4sc5ccccc5c34)n2)cc1 ; c1ccc(-c2nc(-c3cc(-c4ccc5oc6ccccc6c5c4)cc4oc5ccccc5c34)nc(-c3cccc4sc5ccccc5c34)n2)cc1 ; c1ccc(-c2ccc3cc(-c4nc(-c5cccc6oc7cc(-c8ccc9c(c8)oc8ccccc89)ccc7c56)nc(-c5cccc6sc7ccccc7c56)n4)ccc3c2)cc1]

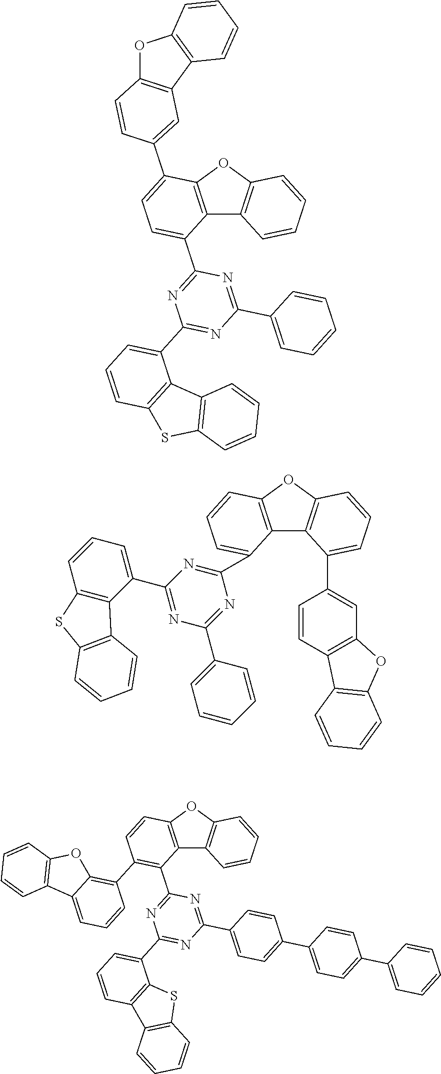

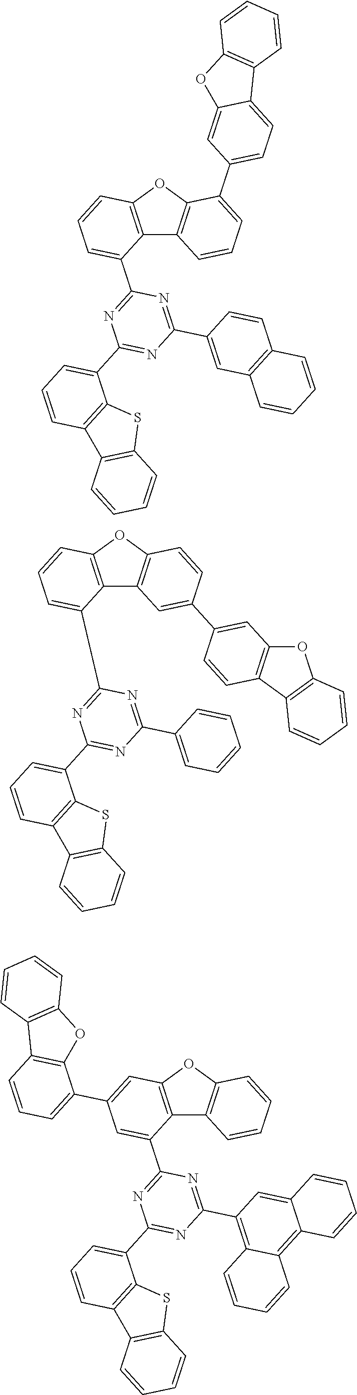

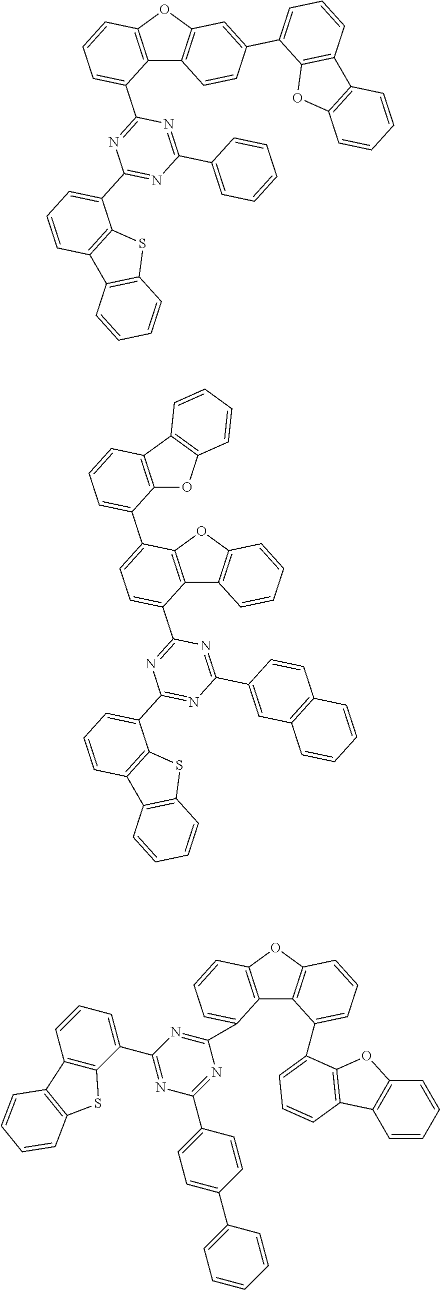 [SMILES: c1ccc(-c2nc(-c3cccc4c3sc3ccccc34)nc(-c3cccc4oc5cc(-c6cccc7c6oc6ccccc67)ccc5c34)n2)cc1 ; c1ccc2cc(-c3nc(-c4cccc5c4sc4ccccc45)nc(-c4ccc(-c5cccc6c5oc5ccccc56)c5oc6ccccc6c45)n3)ccc2c1 ; c1ccc(-c2ccc(-c3nc(-c4cccc5c4sc4ccccc45)nc(-c4cccc5oc6cccc(-c7cccc8c7oc7ccccc78)c6c45)n3)cc2)cc1]

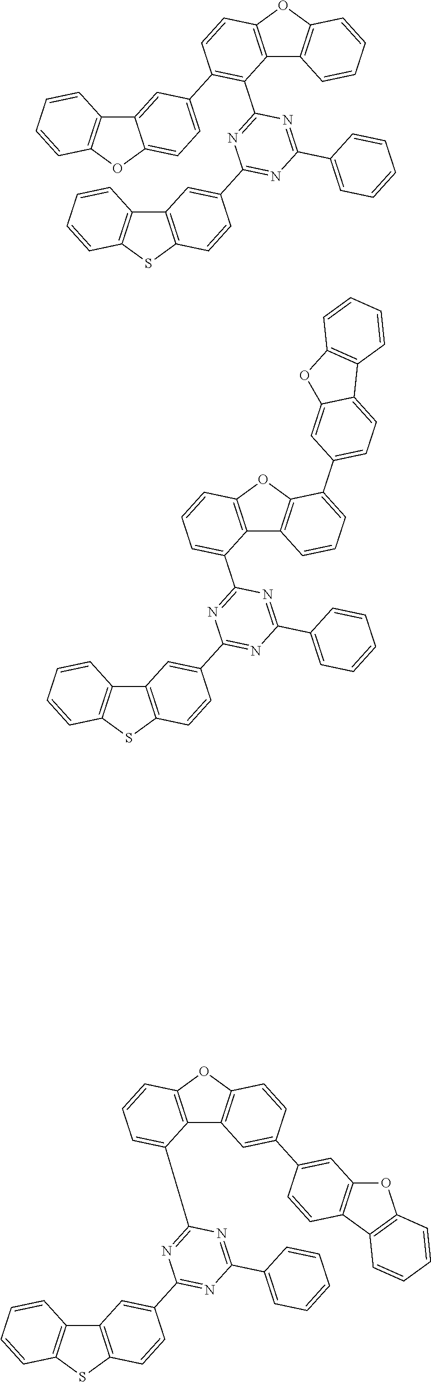 [SMILES: c1ccc(-c2nc(-c3ccc4sc5ccccc5c4c3)nc(-c3c(-c4ccc5oc6ccccc6c5c4)ccc4oc5ccccc5c34)n2)cc1 ; c1ccc(-c2nc(-c3ccc4sc5ccccc5c4c3)nc(-c3cccc4oc5c(-c6ccc7c(c6)oc6ccccc67)cccc5c34)n2)cc1 ; c1ccc(-c2nc(-c3ccc4sc5ccccc5c4c3)nc(-c3cccc4oc5ccc(-c6ccc7c(c6)oc6ccccc67)cc5c34)n2)cc1]

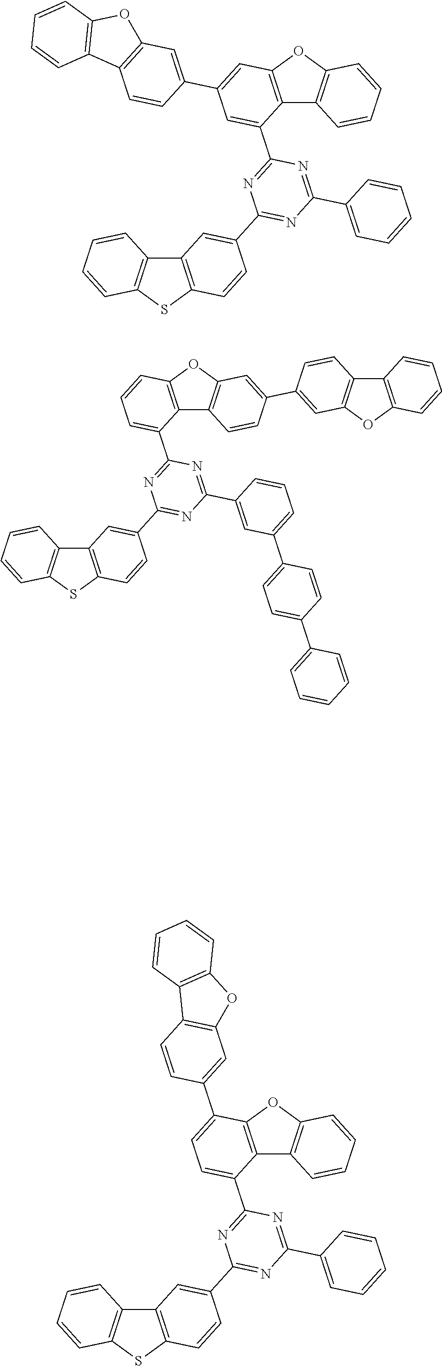 [SMILES: c1ccc(-c2nc(-c3ccc4sc5ccccc5c4c3)nc(-c3cc(-c4ccc5c(c4)oc4ccccc45)cc4oc5ccccc5c34)n2)cc1 ; c1ccc(-c2ccc(-c3cccc(-c4nc(-c5ccc6sc7ccccc7c6c5)nc(-c5cccc6oc7cc(-c8ccc9c(c8)oc8ccccc89)ccc7c56)n4)c3)cc2)cc1 ; c1ccc(-c2nc(-c3ccc4sc5ccccc5c4c3)nc(-c3ccc(-c4ccc5c(c4)oc4ccccc45)c4oc5ccccc5c34)n2)cc1]

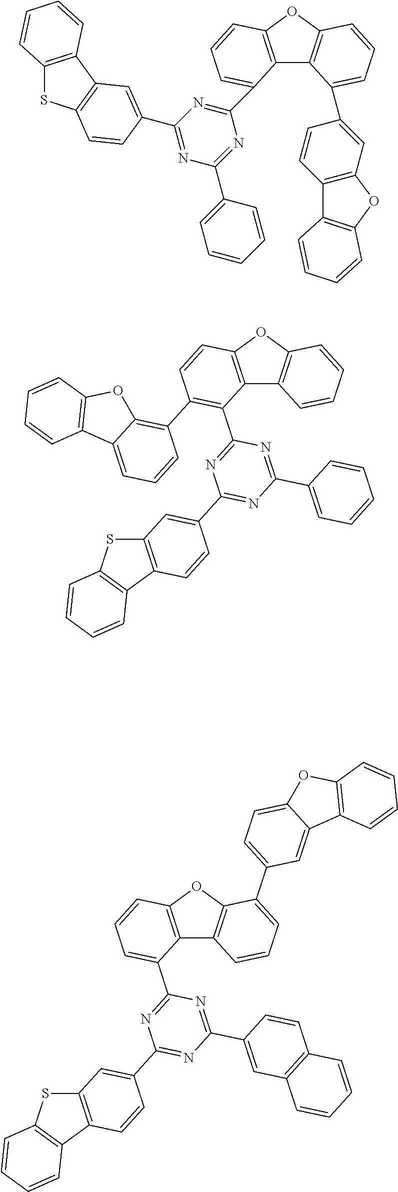 [SMILES: c1ccc(-c2nc(-c3ccc4sc5ccccc5c4c3)nc(-c3cccc4oc5cccc(-c6ccc7c(c6)oc6ccccc67)c5c34)n2)cc1 ; c1ccc(-c2nc(-c3ccc4c(c3)sc3ccccc34)nc(-c3c(-c4cccc5c4oc4ccccc45)ccc4oc5ccccc5c34)n2)cc1 ; c1ccc2cc(-c3nc(-c4ccc5c(c4)sc4ccccc45)nc(-c4cccc5oc6c(-c7ccc8oc9ccccc9c8c7)cccc6c45)n3)ccc2c1]

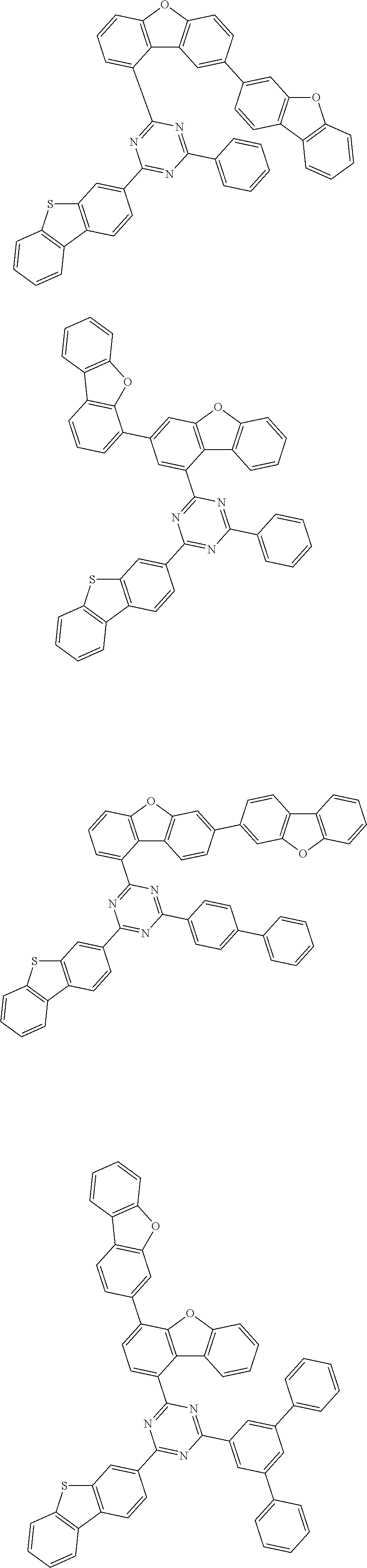 [SMILES: c1ccc(-c2nc(-c3ccc4c(c3)sc3ccccc34)nc(-c3cccc4oc5ccc(-c6ccc7c(c6)oc6ccccc67)cc5c34)n2)cc1 ; c1ccc(-c2nc(-c3ccc4c(c3)sc3ccccc34)nc(-c3cc(-c4cccc5c4oc4ccccc45)cc4oc5ccccc5c34)n2)cc1 ; c1ccc(-c2ccc(-c3nc(-c4ccc5c(c4)sc4ccccc45)nc(-c4cccc5oc6cc(-c7ccc8c(c7)oc7ccccc78)ccc6c45)n3)cc2)cc1 ; c1ccc(-c2cc(-c3ccccc3)cc(-c3nc(-c4ccc5c(c4)sc4ccccc45)nc(-c4ccc(-c5ccc6c(c5)oc5ccccc56)c5oc6ccccc6c45)n3)c2)cc1]

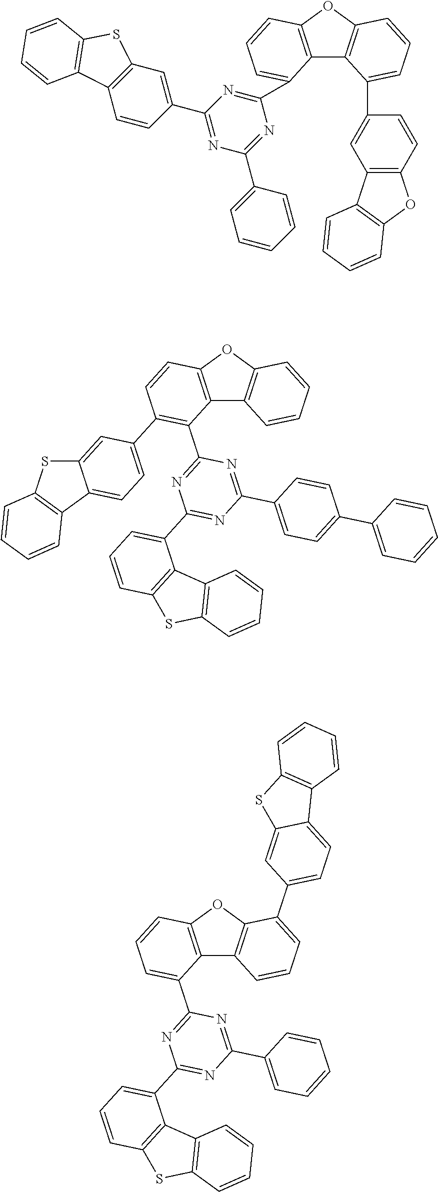 [SMILES: c1ccc(-c2nc(-c3ccc4c(c3)sc3ccccc34)nc(-c3cccc4oc5cccc(-c6ccc7oc8ccccc8c7c6)c5c34)n2)cc1 ; c1ccc(-c2ccc(-c3nc(-c4c(-c5ccc6c(c5)sc5ccccc56)ccc5oc6ccccc6c45)nc(-c4cccc5sc6ccccc6c45)n3)cc2)cc1 ; c1ccc(-c2nc(-c3cccc4oc5c(-c6ccc7c(c6)sc6ccccc67)cccc5c34)nc(-c3cccc4sc5ccccc5c34)n2)cc1]

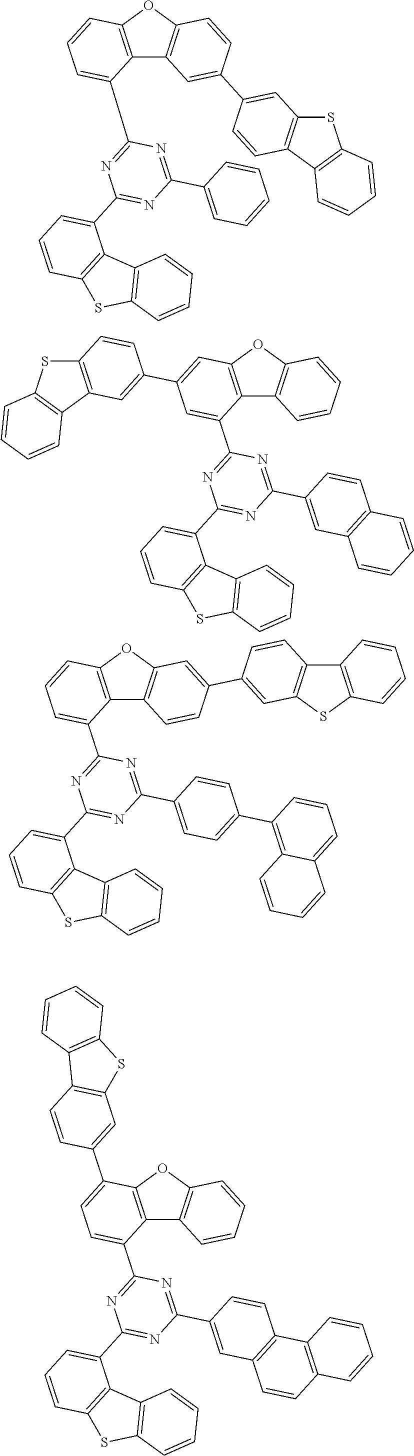

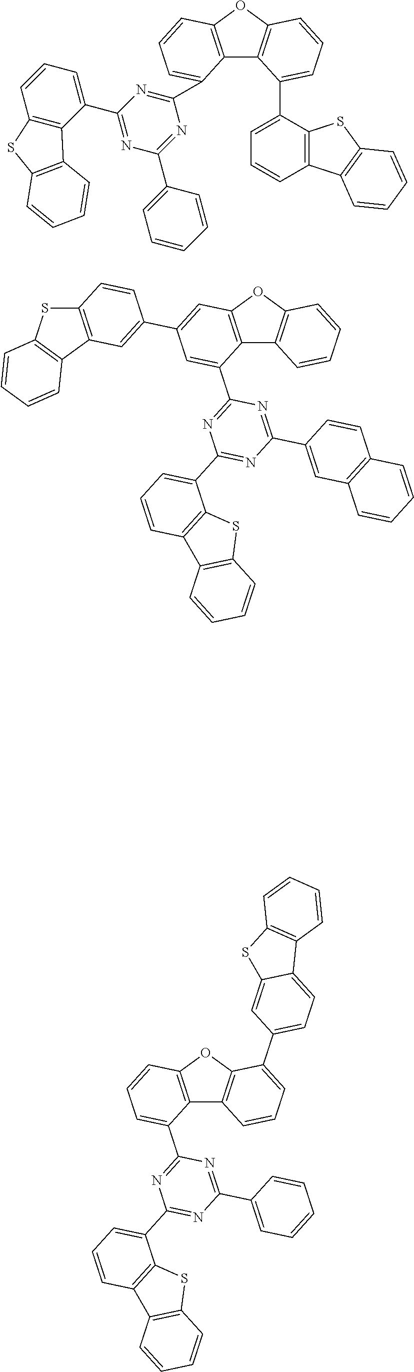 [SMILES: c1ccc(-c2nc(-c3cccc4sc5ccccc5c34)nc(-c3cccc4oc5cccc(-c6cccc7c6sc6ccccc67)c5c34)n2)cc1 ; c1ccc2cc(-c3nc(-c4cccc5c4sc4ccccc45)nc(-c4cc(-c5ccc6sc7ccccc7c6c5)cc5oc6ccccc6c45)n3)ccc2c1 ; c1ccc(-c2nc(-c3cccc4c3sc3ccccc34)nc(-c3cccc4oc5c(-c6ccc7c(c6)sc6ccccc67)cccc5c34)n2)cc1]

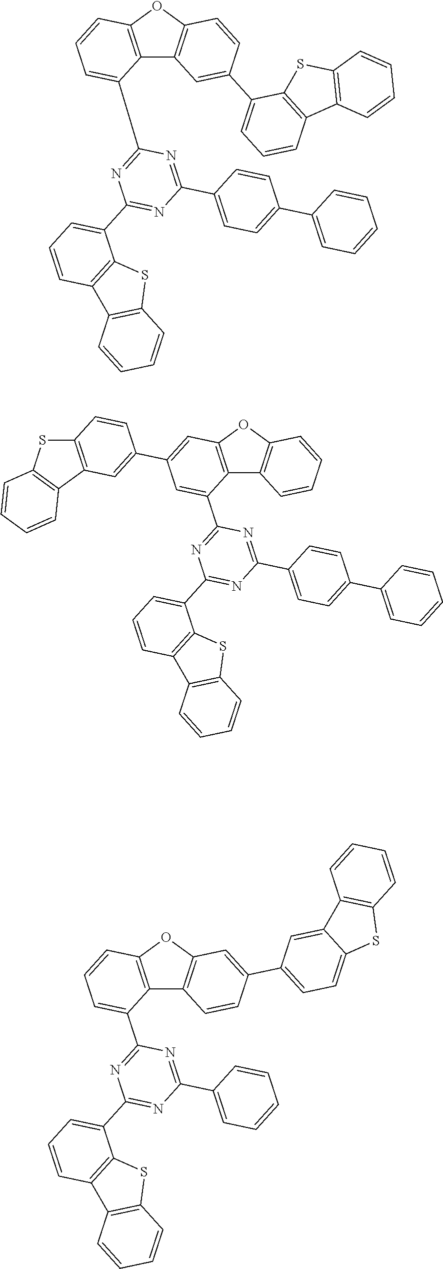 [SMILES: c1ccc(-c2ccc(-c3nc(-c4cccc5c4sc4ccccc45)nc(-c4cccc5oc6ccc(-c7cccc8c7sc7ccccc78)cc6c45)n3)cc2)cc1 ; c1ccc(-c2ccc(-c3nc(-c4cccc5c4sc4ccccc45)nc(-c4cc(-c5ccc6sc7ccccc7c6c5)cc5oc6ccccc6c45)n3)cc2)cc1 ; c1ccc(-c2nc(-c3cccc4c3sc3ccccc34)nc(-c3cccc4oc5cc(-c6ccc7sc8ccccc8c7c6)ccc5c34)n2)cc1]

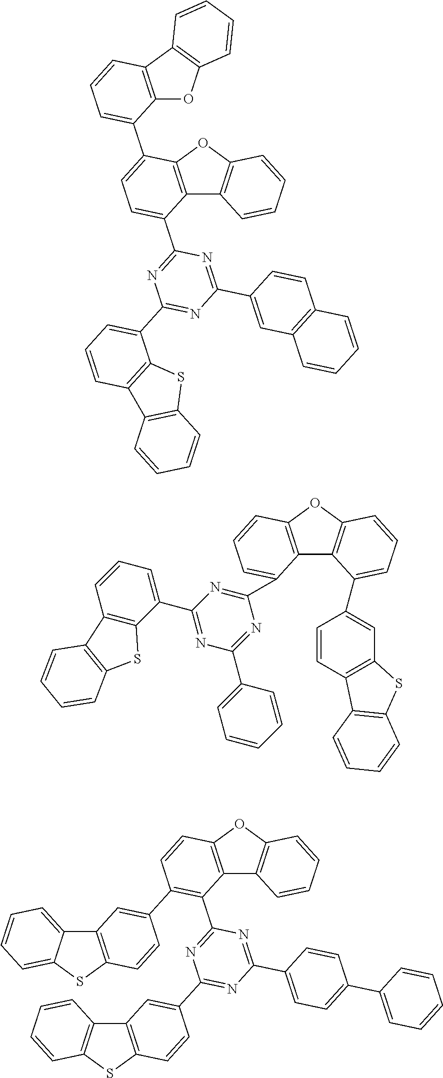 [SMILES: c1ccc2cc(-c3nc(-c4cccc5c4sc4ccccc45)nc(-c4ccc(-c5cccc6c5oc5ccccc56)c5oc6ccccc6c45)n3)ccc2c1 ; c1ccc(-c2nc(-c3cccc4c3sc3ccccc34)nc(-c3cccc4oc5cccc(-c6ccc7c(c6)sc6ccccc67)c5c34)n2)cc1 ; c1ccc(-c2ccc(-c3nc(-c4ccc5sc6ccccc6c5c4)nc(-c4c(-c5ccc6sc7ccccc7c6c5)ccc5oc6ccccc6c45)n3)cc2)cc1]

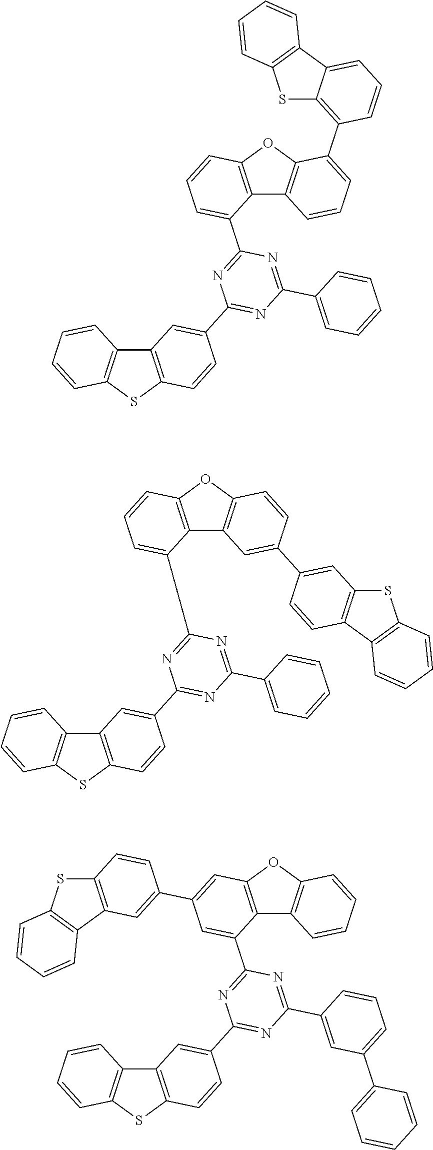 [SMILES: c1ccc(-c2nc(-c3ccc4sc5ccccc5c4c3)nc(-c3cccc4oc5c(-c6cccc7c6sc6ccccc67)cccc5c34)n2)cc1 ; c1ccc(-c2nc(-c3ccc4sc5ccccc5c4c3)nc(-c3cccc4oc5ccc(-c6ccc7c(c6)sc6ccccc67)cc5c34)n2)cc1 ; c1ccc(-c2cccc(-c3nc(-c4ccc5sc6ccccc6c5c4)nc(-c4cc(-c5ccc6sc7ccccc7c6c5)cc5oc6ccccc6c45)n3)c2)cc1]

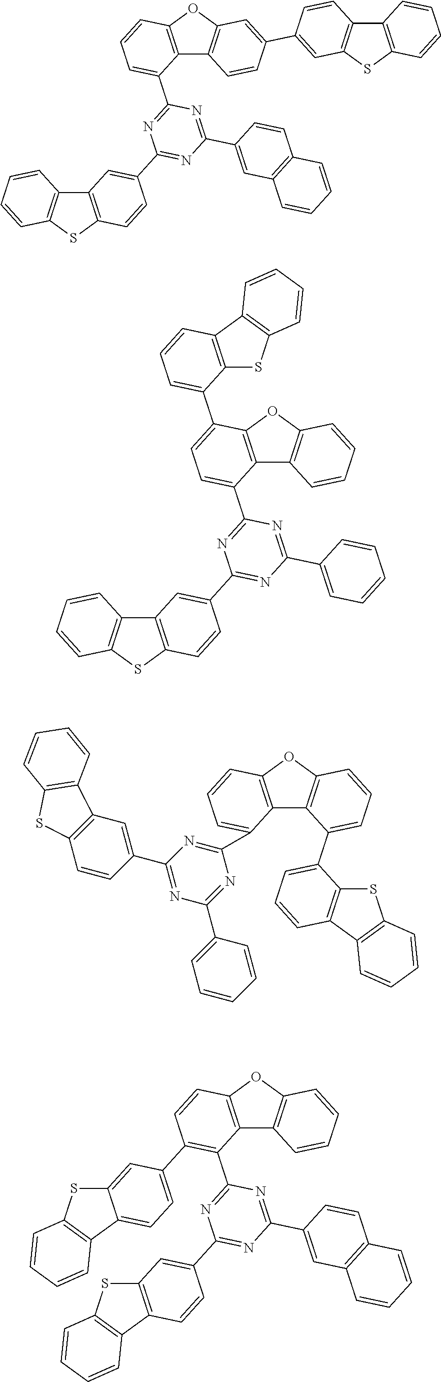 [SMILES: c1ccc2cc(-c3nc(-c4ccc5sc6ccccc6c5c4)nc(-c4cccc5oc6cc(-c7ccc8c(c7)sc7ccccc78)ccc6c45)n3)ccc2c1 ; c1ccc(-c2nc(-c3ccc4sc5ccccc5c4c3)nc(-c3ccc(-c4cccc5c4sc4ccccc45)c4oc5ccccc5c34)n2)cc1 ; c1ccc(-c2nc(-c3ccc4sc5ccccc5c4c3)nc(-c3cccc4oc5cccc(-c6cccc7c6sc6ccccc67)c5c34)n2)cc1 ; c1ccc2cc(-c3nc(-c4ccc5c(c4)sc4ccccc45)nc(-c4c(-c5ccc6c(c5)sc5ccccc56)ccc5oc6ccccc6c45)n3)ccc2c1]

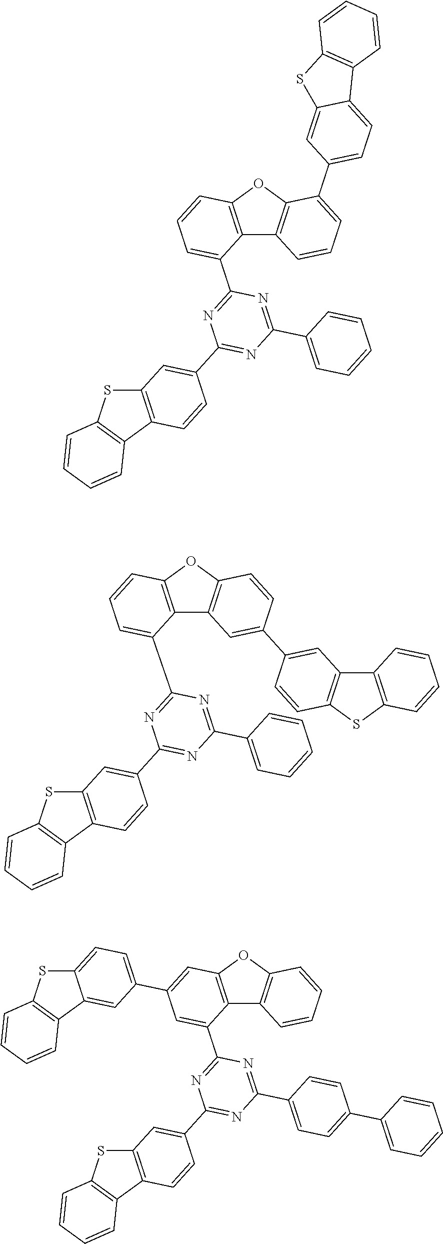 [SMILES: c1ccc(-c2nc(-c3ccc4c(c3)sc3ccccc34)nc(-c3cccc4oc5c(-c6ccc7c(c6)sc6ccccc67)cccc5c34)n2)cc1 ; c1ccc(-c2nc(-c3ccc4c(c3)sc3ccccc34)nc(-c3cccc4oc5ccc(-c6ccc7sc8ccccc8c7c6)cc5c34)n2)cc1 ; c1ccc(-c2ccc(-c3nc(-c4ccc5c(c4)sc4ccccc45)nc(-c4cc(-c5ccc6sc7ccccc7c6c5)cc5oc6ccccc6c45)n3)cc2)cc1]

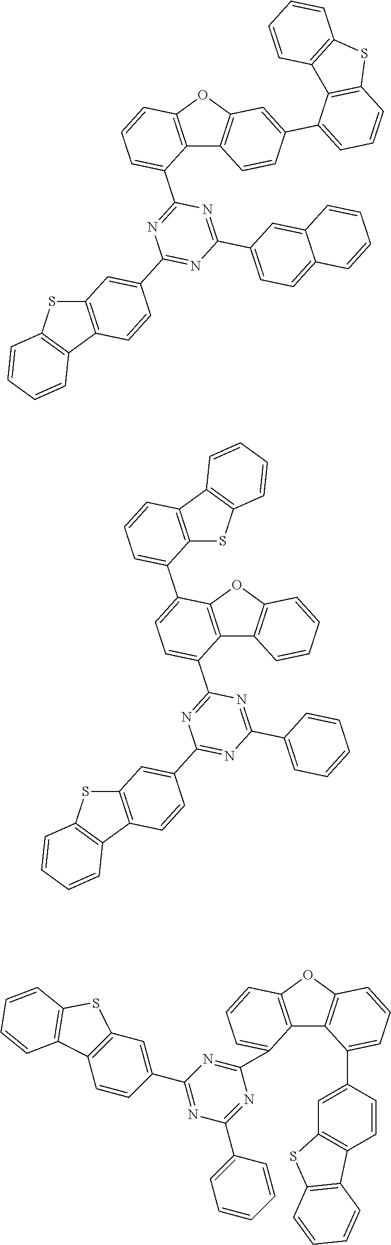 [SMILES: c1ccc2cc(-c3nc(-c4ccc5c(c4)sc4ccccc45)nc(-c4cccc5oc6cc(-c7cccc8sc9ccccc9c78)ccc6c45)n3)ccc2c1 ; c1ccc(-c2nc(-c3ccc4c(c3)sc3ccccc34)nc(-c3ccc(-c4cccc5c4sc4ccccc45)c4oc5ccccc5c34)n2)cc1 ; c1ccc(-c2nc(-c3ccc4c(c3)sc3ccccc34)nc(-c3cccc4oc5cccc(-c6ccc7c(c6)sc6ccccc67)c5c34)n2)cc1]

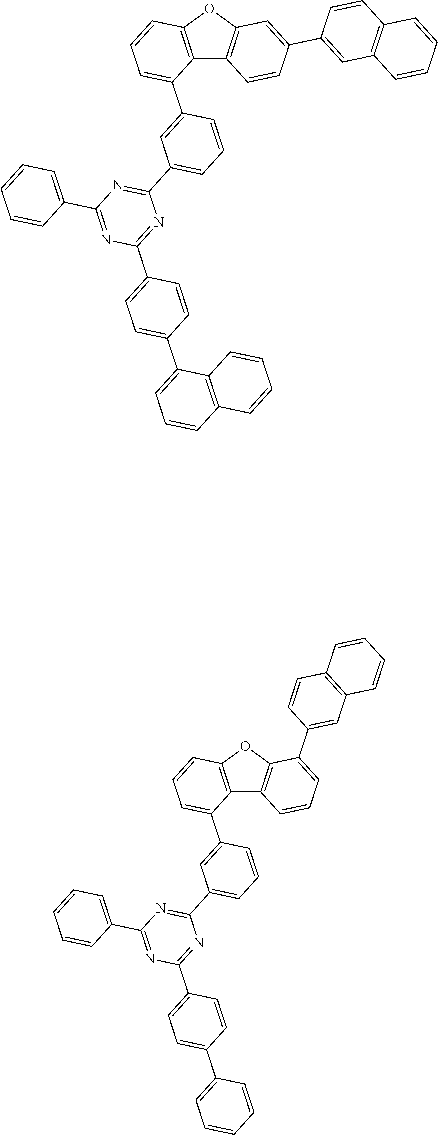 [SMILES: c1ccc(-c2nc(-c3ccc(-c4cccc5ccccc45)cc3)nc(-c3cccc(-c4cccc5oc6cc(-c7ccc8ccccc8c7)ccc6c45)c3)n2)cc1 ; c1ccc(-c2ccc(-c3nc(-c4ccccc4)nc(-c4cccc(-c5cccc6oc7c(-c8ccc9ccccc9c8)cccc7c56)c4)n3)cc2)cc1]

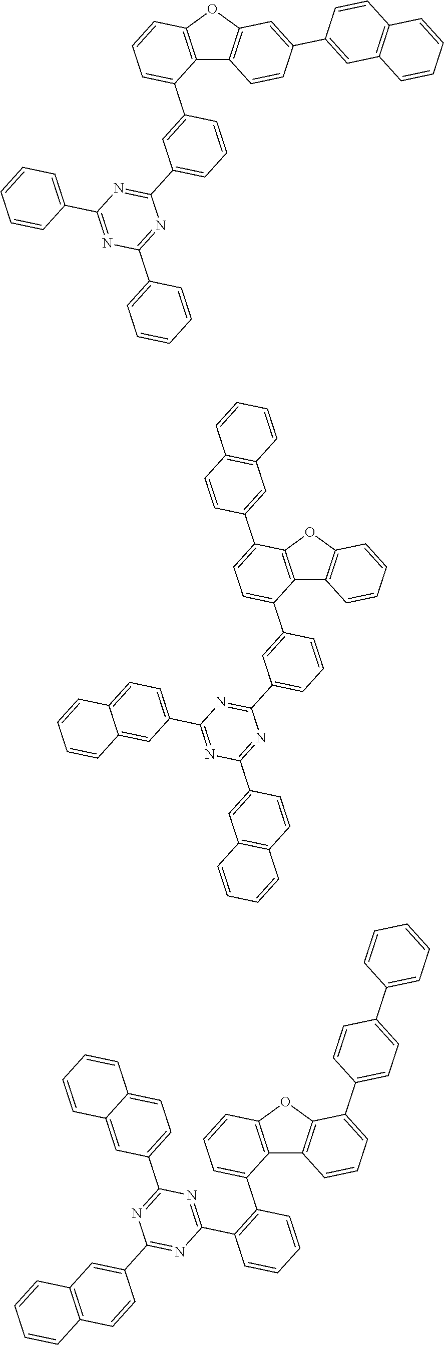 [SMILES: c1ccc(-c2nc(-c3ccccc3)nc(-c3cccc(-c4cccc5oc6cc(-c7ccc8ccccc8c7)ccc6c45)c3)n2)cc1 ; c1cc(-c2nc(-c3ccc4ccccc4c3)nc(-c3ccc4ccccc4c3)n2)cc(-c2ccc(-c3ccc4ccccc4c3)c3oc4ccccc4c23)c1 ; c1ccc(-c2ccc(-c3cccc4c3oc3cccc(-c5ccccc5-c5nc(-c6ccc7ccccc7c6)nc(-c6ccc7ccccc7c6)n5)c34)cc2)cc1]

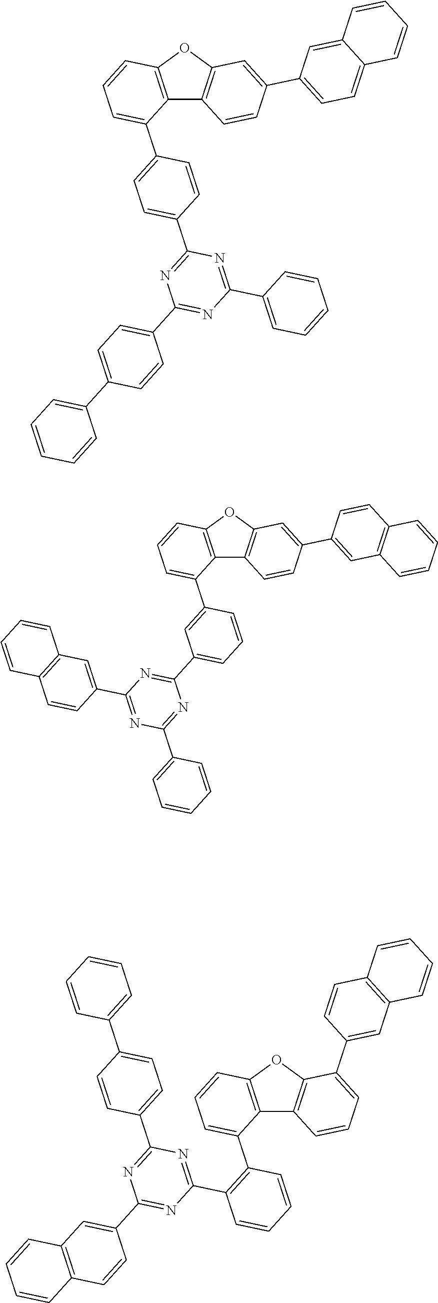 [SMILES: c1ccc(-c2ccc(-c3nc(-c4ccccc4)nc(-c4ccc(-c5cccc6oc7cc(-c8ccc9ccccc9c8)ccc7c56)cc4)n3)cc2)cc1 ; c1ccc(-c2nc(-c3cccc(-c4cccc5oc6cc(-c7ccc8ccccc8c7)ccc6c45)c3)nc(-c3ccc4ccccc4c3)n2)cc1 ; c1ccc(-c2ccc(-c3nc(-c4ccc5ccccc5c4)nc(-c4ccccc4-c4cccc5oc6c(-c7ccc8ccccc8c7)cccc6c45)n3)cc2)cc1]

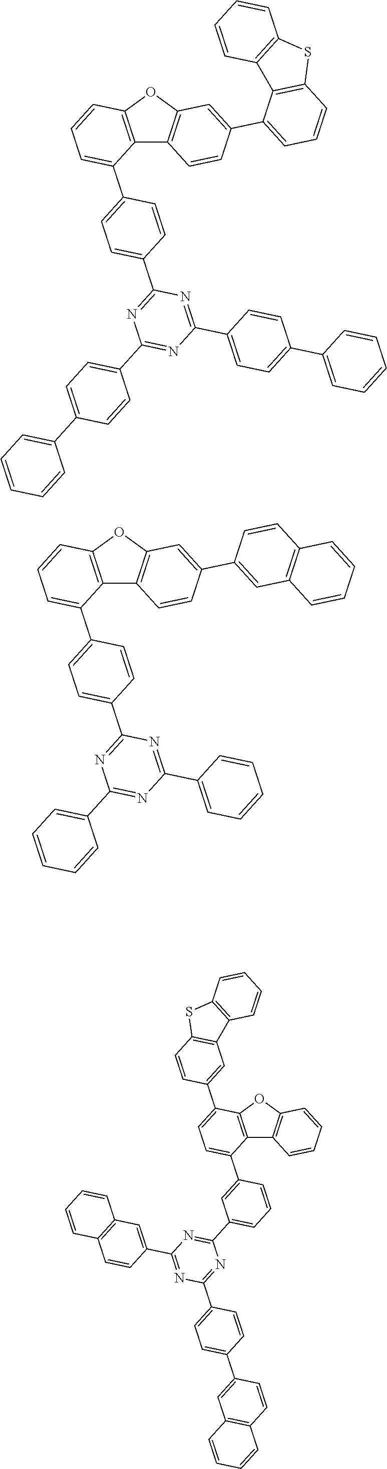 [SMILES: c1ccc(-c2ccc(-c3nc(-c4ccc(-c5ccccc5)cc4)nc(-c4ccc(-c5cccc6oc7cc(-c8cccc9sc%10ccccc%10c89)ccc7c56)cc4)n3)cc2)cc1 ; c1ccc(-c2nc(-c3ccccc3)nc(-c3ccc(-c4cccc5oc6cc(-c7ccc8ccccc8c7)ccc6c45)cc3)n2)cc1 ; c1cc(-c2nc(-c3ccc(-c4ccc5ccccc5c4)cc3)nc(-c3ccc4ccccc4c3)n2)cc(-c2ccc(-c3ccc4sc5ccccc5c4c3)c3oc4ccccc4c23)c1]

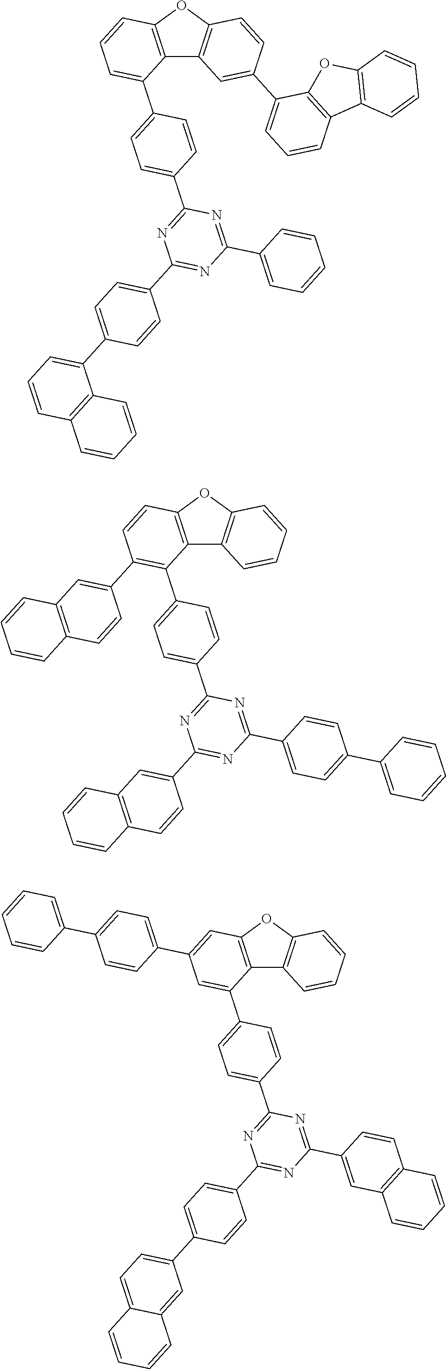 [SMILES: c1ccc(-c2nc(-c3ccc(-c4cccc5ccccc45)cc3)nc(-c3ccc(-c4cccc5oc6ccc(-c7cccc8c7oc7ccccc78)cc6c45)cc3)n2)cc1 ; c1ccc(-c2ccc(-c3nc(-c4ccc(-c5c(-c6ccc7ccccc7c6)ccc6oc7ccccc7c56)cc4)nc(-c4ccc5ccccc5c4)n3)cc2)cc1 ; c1ccc(-c2ccc(-c3cc(-c4ccc(-c5nc(-c6ccc(-c7ccc8ccccc8c7)cc6)nc(-c6ccc7ccccc7c6)n5)cc4)c4c(c3)oc3ccccc34)cc2)cc1]

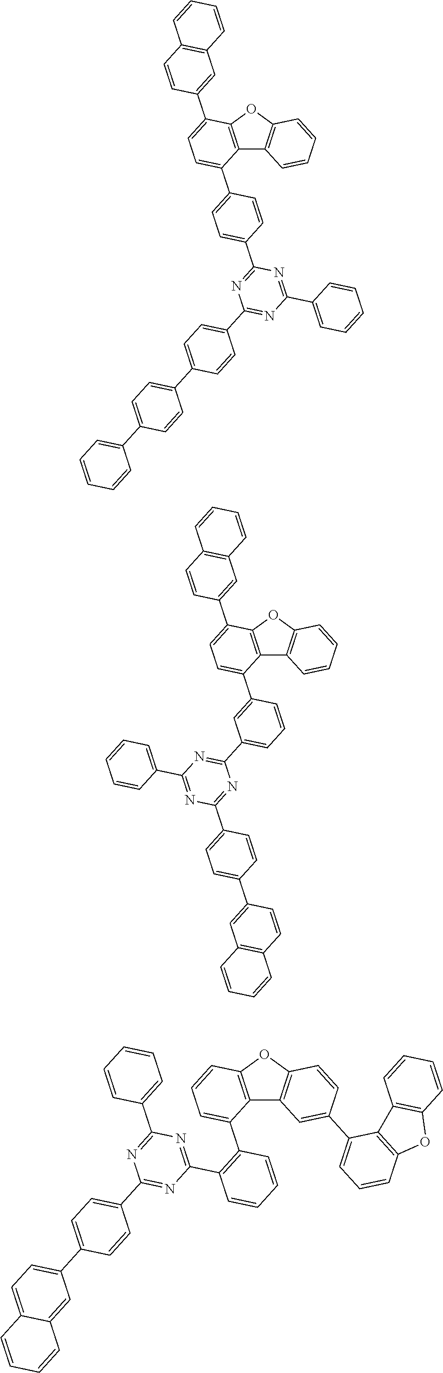 [SMILES: c1ccc(-c2ccc(-c3ccc(-c4nc(-c5ccccc5)nc(-c5ccc(-c6ccc(-c7ccc8ccccc8c7)c7oc8ccccc8c67)cc5)n4)cc3)cc2)cc1 ; c1ccc(-c2nc(-c3ccc(-c4ccc5ccccc5c4)cc3)nc(-c3cccc(-c4ccc(-c5ccc6ccccc6c5)c5oc6ccccc6c45)c3)n2)cc1 ; c1ccc(-c2nc(-c3ccc(-c4ccc5ccccc5c4)cc3)nc(-c3ccccc3-c3cccc4oc5ccc(-c6cccc7oc8ccccc8c67)cc5c34)n2)cc1]

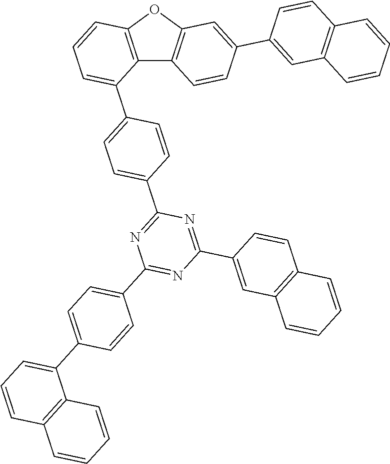 [SMILES: c1ccc2cc(-c3ccc4c(c3)oc3cccc(-c5ccc(-c6nc(-c7ccc(-c8cccc9ccccc89)cc7)nc(-c7ccc8ccccc8c7)n6)cc5)c34)ccc2c1]

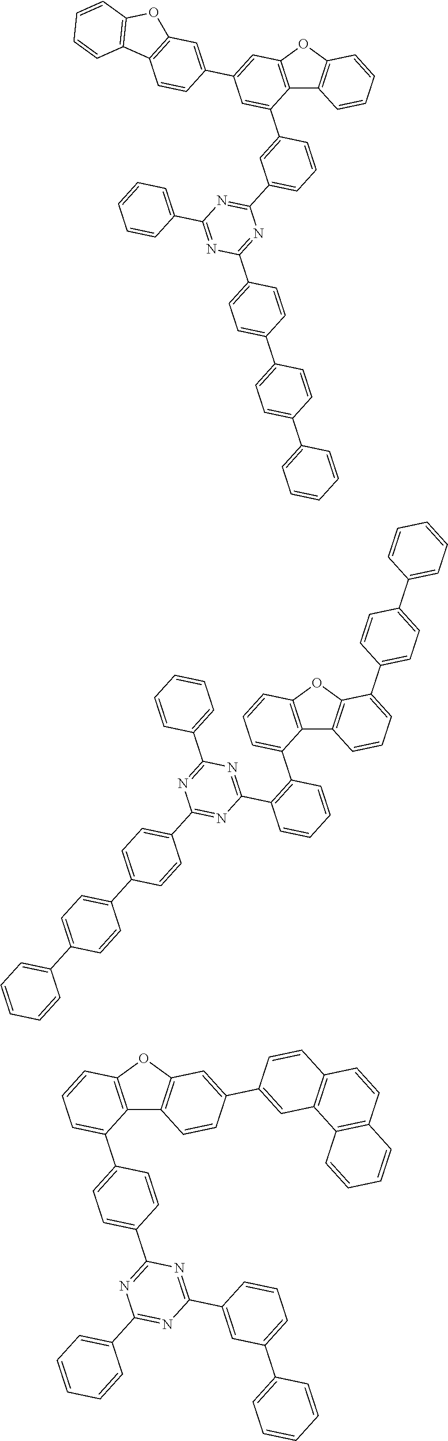 [SMILES: c1ccc(-c2ccc(-c3ccc(-c4nc(-c5ccccc5)nc(-c5cccc(-c6cc(-c7ccc8c(c7)oc7ccccc78)cc7oc8ccccc8c67)c5)n4)cc3)cc2)cc1 ; c1ccc(-c2ccc(-c3ccc(-c4nc(-c5ccccc5)nc(-c5ccccc5-c5cccc6oc7c(-c8ccc(-c9ccccc9)cc8)cccc7c56)n4)cc3)cc2)cc1 ; c1ccc(-c2cccc(-c3nc(-c4ccccc4)nc(-c4ccc(-c5cccc6oc7cc(-c8ccc9ccc%10ccccc%10c9c8)ccc7c56)cc4)n3)c2)cc1]

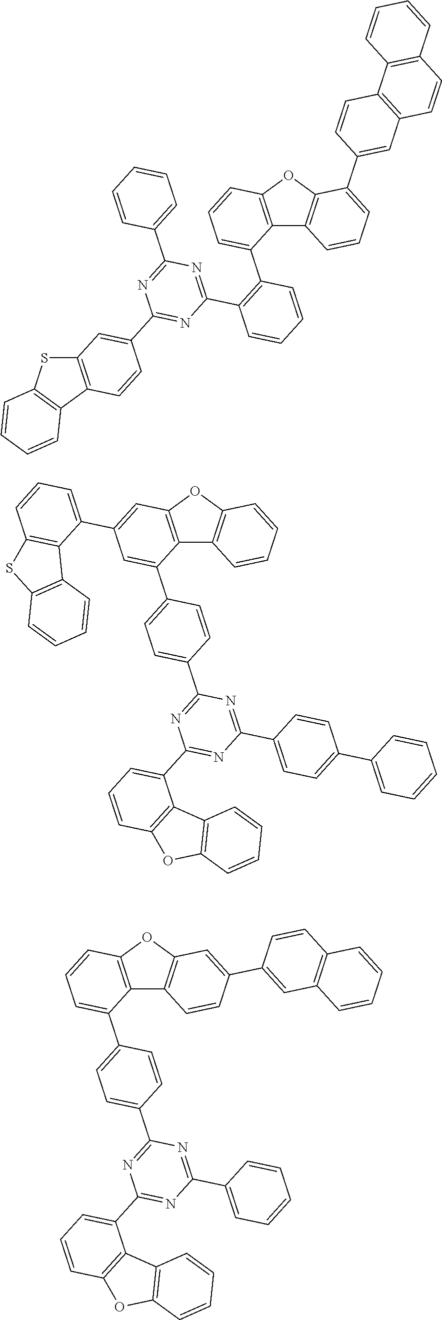

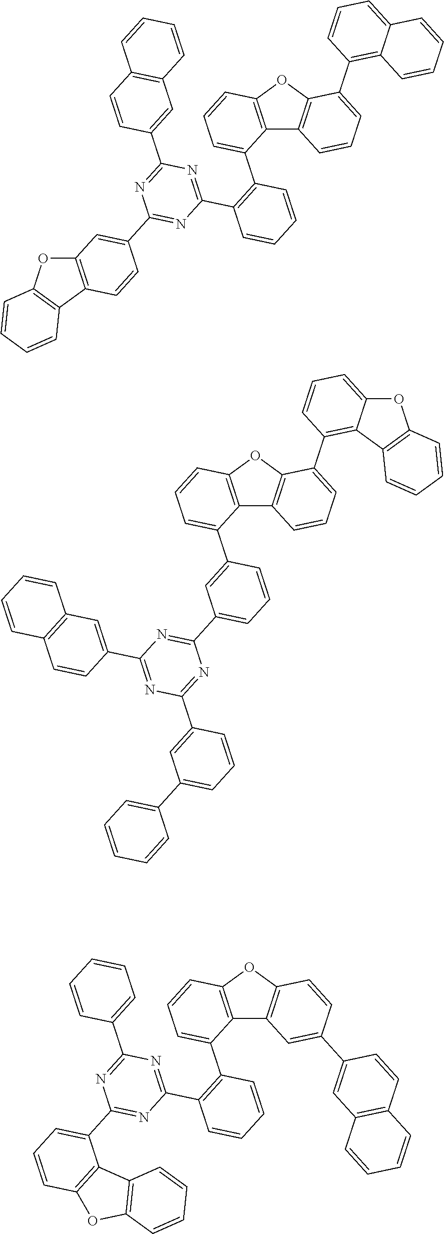 [SMILES: c1ccc(-c2cccc3oc4c(-c5cccc6ccccc56)cccc4c23)c(-c2nc(-c3ccc4ccccc4c3)nc(-c3ccc4c(c3)oc3ccccc34)n2)c1 ; c1ccc(-c2cccc(-c3nc(-c4cccc(-c5cccc6oc7c(-c8cccc9oc%10ccccc%10c89)cccc7c56)c4)nc(-c4ccc5ccccc5c4)n3)c2)cc1 ; c1ccc(-c2nc(-c3ccccc3-c3cccc4oc5ccc(-c6ccc7ccccc7c6)cc5c34)nc(-c3cccc4oc5ccccc5c34)n2)cc1]

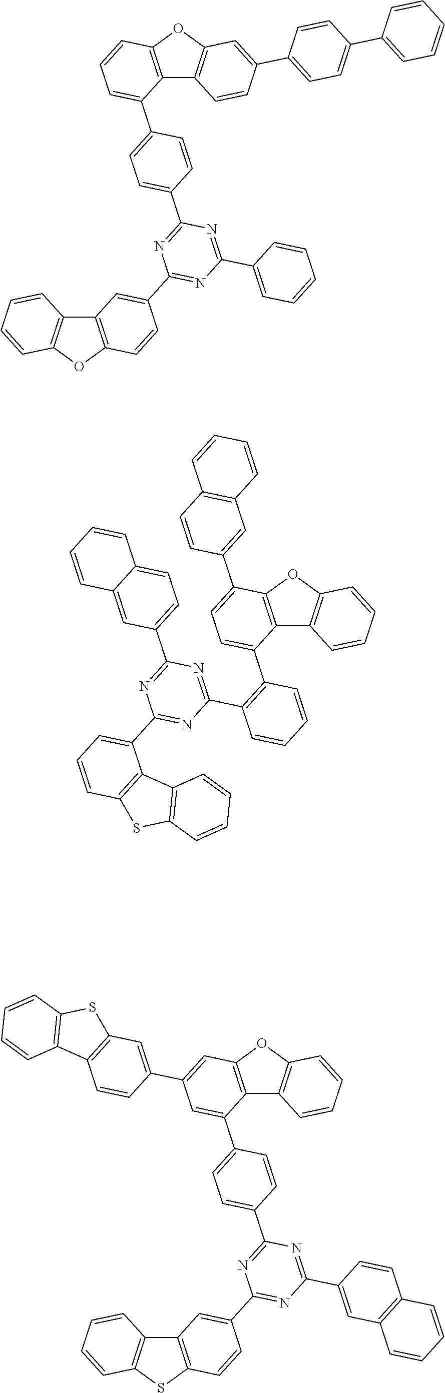 [SMILES: c1ccc(-c2ccc(-c3ccc4c(c3)oc3cccc(-c5ccc(-c6nc(-c7ccccc7)nc(-c7ccc8oc9ccccc9c8c7)n6)cc5)c34)cc2)cc1 ; c1ccc(-c2ccc(-c3ccc4ccccc4c3)c3oc4ccccc4c23)c(-c2nc(-c3ccc4ccccc4c3)nc(-c3cccc4sc5ccccc5c34)n2)c1 ; c1ccc2cc(-c3nc(-c4ccc(-c5cc(-c6ccc7c(c6)sc6ccccc67)cc6oc7ccccc7c56)cc4)nc(-c4ccc5sc6ccccc6c5c4)n3)ccc2c1]

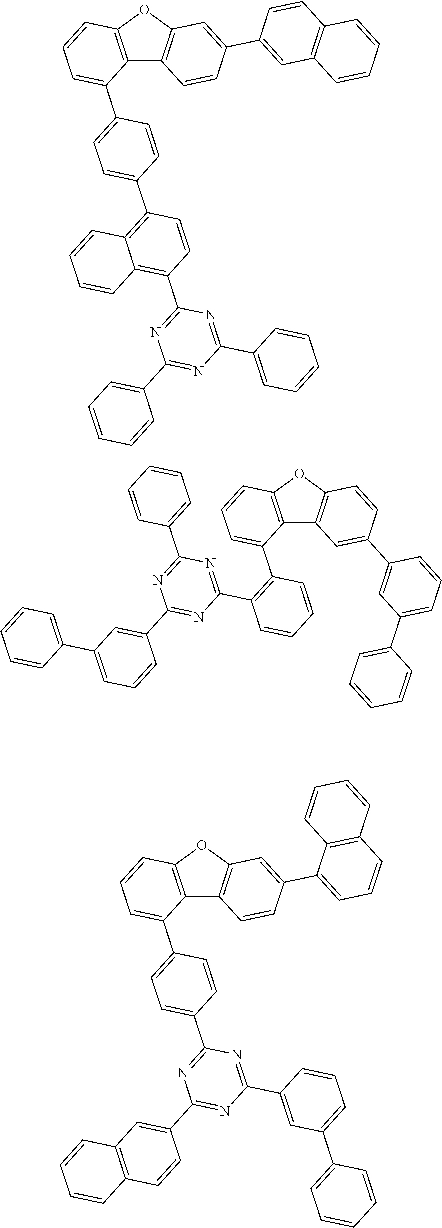

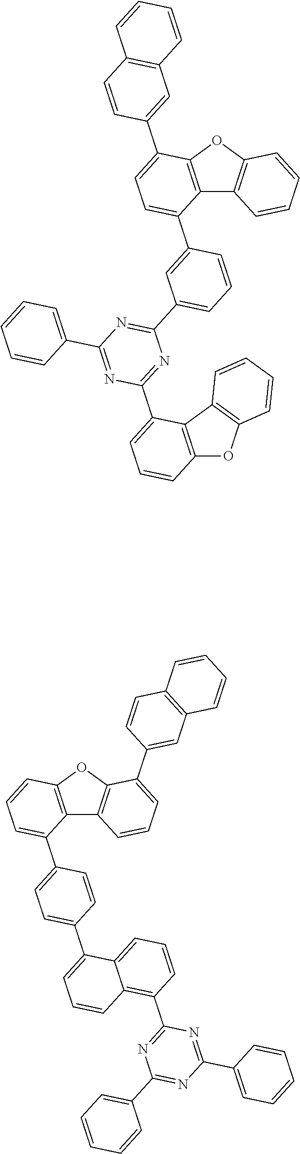 [SMILES: c1ccc(-c2nc(-c3cccc(-c4ccc(-c5ccc6ccccc6c5)c5oc6ccccc6c45)c3)nc(-c3cccc4oc5ccccc5c34)n2)cc1 ; c1ccc(-c2nc(-c3ccccc3)nc(-c3cccc4c(-c5ccc(-c6cccc7oc8c(-c9ccc%10ccccc%10c9)cccc8c67)cc5)cccc34)n2)cc1]

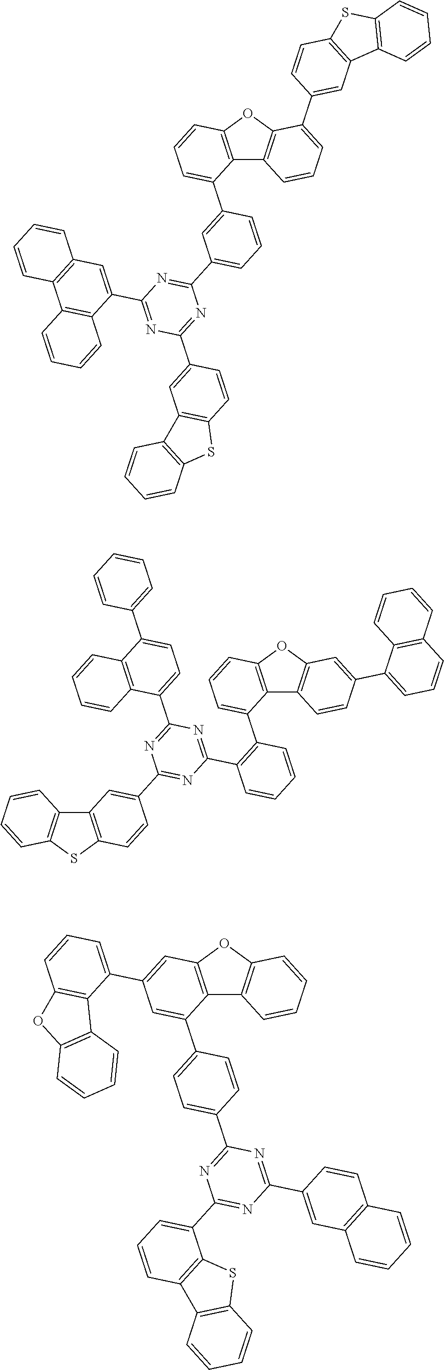 [SMILES: c1cc(-c2nc(-c3ccc4sc5ccccc5c4c3)nc(-c3cc4ccccc4c4ccccc34)n2)cc(-c2cccc3oc4c(-c5ccc6sc7ccccc7c6c5)cccc4c23)c1 ; c1ccc(-c2ccc(-c3nc(-c4ccc5sc6ccccc6c5c4)nc(-c4ccccc4-c4cccc5oc6cc(-c7cccc8ccccc78)ccc6c45)n3)c3ccccc23)cc1 ; c1ccc2cc(-c3nc(-c4ccc(-c5cc(-c6cccc7oc8ccccc8c67)cc6oc7ccccc7c56)cc4)nc(-c4cccc5c4sc4ccccc45)n3)ccc2c1]

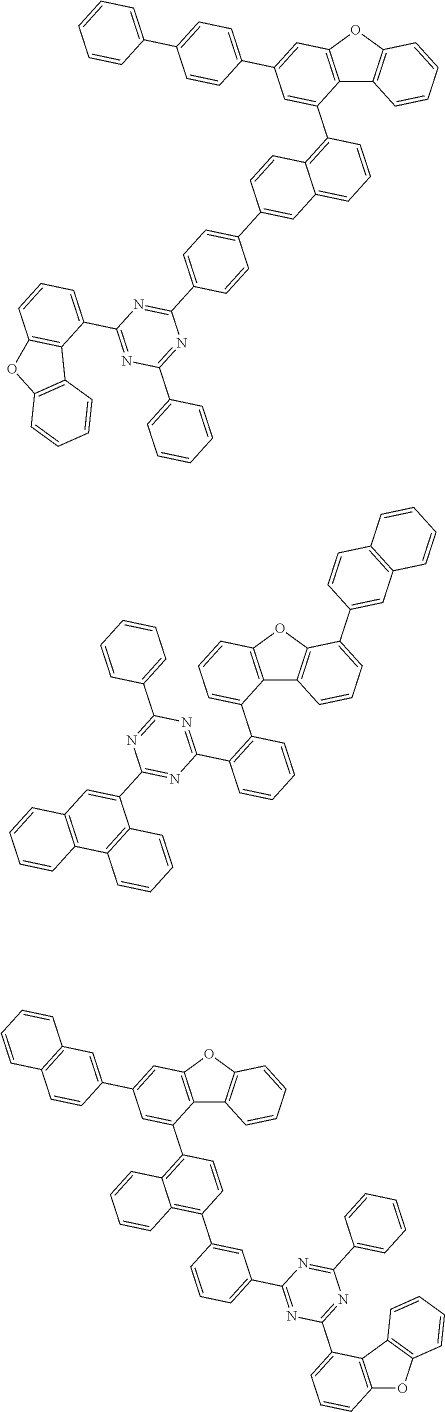 [SMILES: c1ccc(-c2ccc(-c3cc(-c4cccc5cc(-c6ccc(-c7nc(-c8ccccc8)nc(-c8cccc9oc%10ccccc%10c89)n7)cc6)ccc45)c4c(c3)oc3ccccc34)cc2)cc1 ; c1ccc(-c2nc(-c3ccccc3-c3cccc4oc5c(-c6ccc7ccccc7c6)cccc5c34)nc(-c3cc4ccccc4c4ccccc34)n2)cc1 ; c1ccc(-c2nc(-c3cccc(-c4ccc(-c5cc(-c6ccc7ccccc7c6)cc6oc7ccccc7c56)c5ccccc45)c3)nc(-c3cccc4oc5ccccc5c34)n2)cc1]

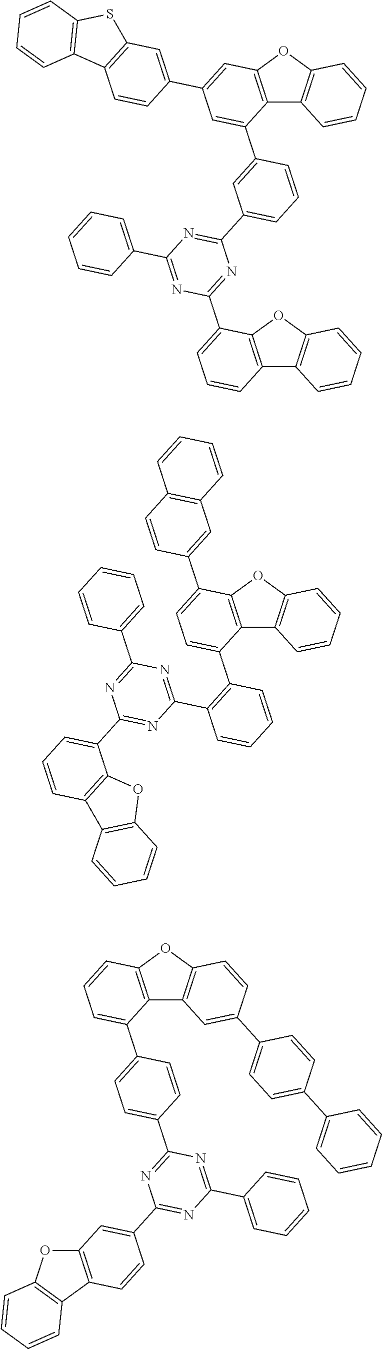 [SMILES: c1ccc(-c2nc(-c3cccc(-c4cc(-c5ccc6c(c5)sc5ccccc56)cc5oc6ccccc6c45)c3)nc(-c3cccc4c3oc3ccccc34)n2)cc1 ; c1ccc(-c2nc(-c3ccccc3-c3ccc(-c4ccc5ccccc5c4)c4oc5ccccc5c34)nc(-c3cccc4c3oc3ccccc34)n2)cc1 ; c1ccc(-c2ccc(-c3ccc4oc5cccc(-c6ccc(-c7nc(-c8ccccc8)nc(-c8ccc9c(c8)oc8ccccc89)n7)cc6)c5c4c3)cc2)cc1]

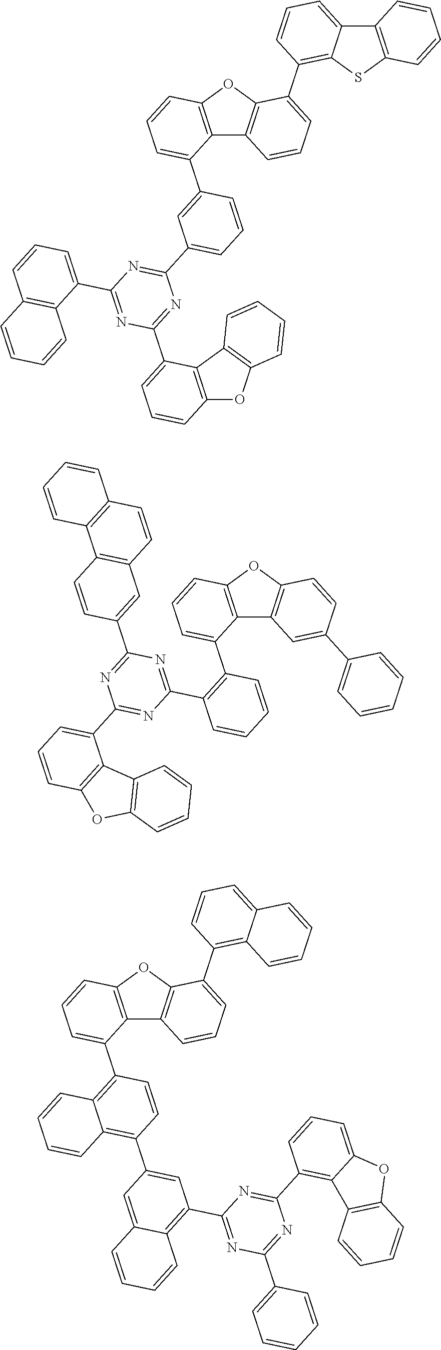 [SMILES: c1cc(-c2nc(-c3cccc4ccccc34)nc(-c3cccc4oc5ccccc5c34)n2)cc(-c2cccc3oc4c(-c5cccc6c5sc5ccccc56)cccc4c23)c1 ; c1ccc(-c2ccc3oc4cccc(-c5ccccc5-c5nc(-c6ccc7c(ccc8ccccc87)c6)nc(-c6cccc7oc8ccccc8c67)n5)c4c3c2)cc1 ; c1ccc(-c2nc(-c3cc(-c4ccc(-c5cccc6oc7c(-c8cccc9ccccc89)cccc7c56)c5ccccc45)cc4ccccc34)nc(-c3cccc4oc5ccccc5c34)n2)cc1]

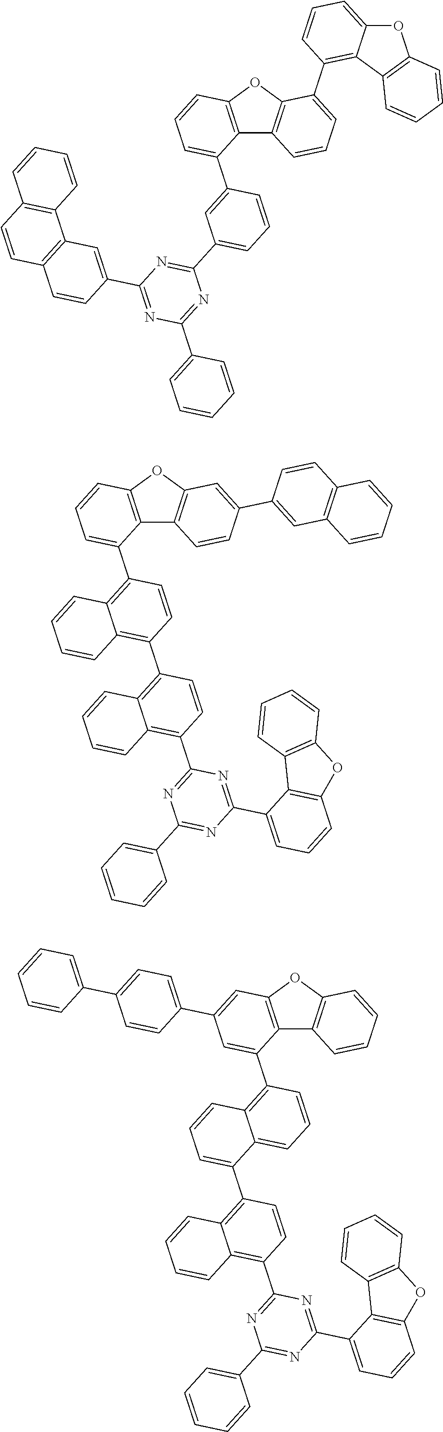 [SMILES: c1ccc(-c2nc(-c3cccc(-c4cccc5oc6c(-c7cccc8oc9ccccc9c78)cccc6c45)c3)nc(-c3ccc4ccc5ccccc5c4c3)n2)cc1 ; c1ccc(-c2nc(-c3ccc(-c4ccc(-c5cccc6oc7cc(-c8ccc9ccccc9c8)ccc7c56)c5ccccc45)c4ccccc34)nc(-c3cccc4oc5ccccc5c34)n2)cc1 ; c1ccc(-c2ccc(-c3cc(-c4cccc5c(-c6ccc(-c7nc(-c8ccccc8)nc(-c8cccc9oc%10ccccc%10c89)n7)c7ccccc67)cccc45)c4c(c3)oc3ccccc34)cc2)cc1]

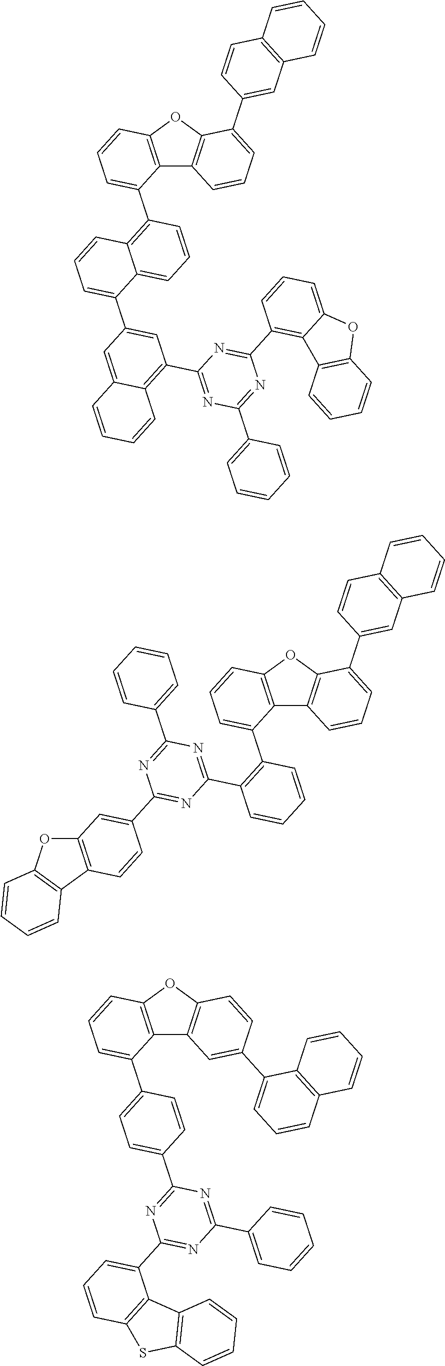 [SMILES: c1ccc(-c2nc(-c3cc(-c4cccc5c(-c6cccc7oc8c(-c9ccc%10ccccc%10c9)cccc8c67)cccc45)cc4ccccc34)nc(-c3cccc4oc5ccccc5c34)n2)cc1 ; c1ccc(-c2nc(-c3ccc4c(c3)oc3ccccc34)nc(-c3ccccc3-c3cccc4oc5c(-c6ccc7ccccc7c6)cccc5c34)n2)cc1 ; c1ccc(-c2nc(-c3ccc(-c4cccc5oc6ccc(-c7cccc8ccccc78)cc6c45)cc3)nc(-c3cccc4sc5ccccc5c34)n2)cc1]

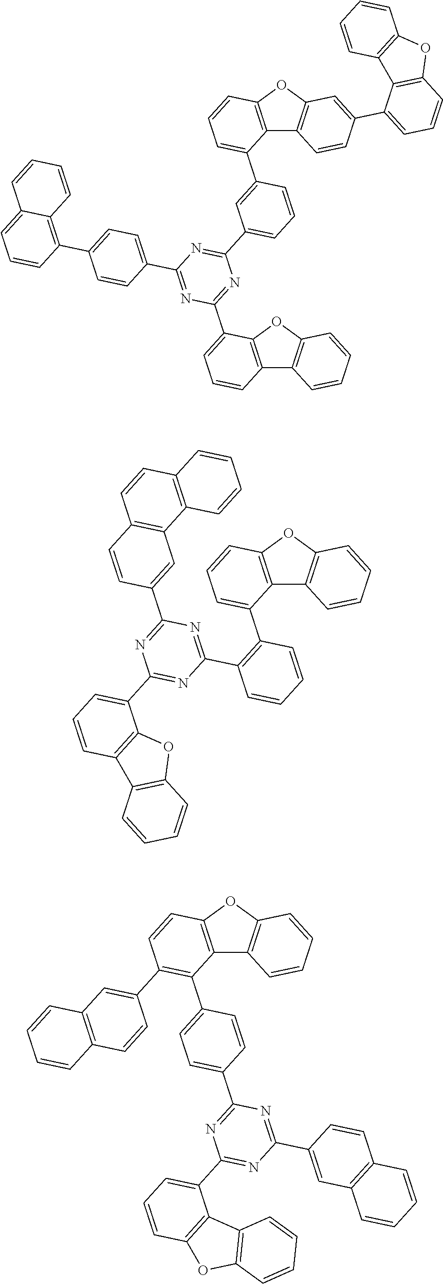 [SMILES: c1cc(-c2nc(-c3ccc(-c4cccc5ccccc45)cc3)nc(-c3cccc4c3oc3ccccc34)n2)cc(-c2cccc3oc4cc(-c5cccc6oc7ccccc7c56)ccc4c23)c1 ; c1ccc(-c2cccc3oc4ccccc4c23)c(-c2nc(-c3ccc4ccc5ccccc5c4c3)nc(-c3cccc4c3oc3ccccc34)n2)c1 ; c1ccc2cc(-c3nc(-c4ccc(-c5c(-c6ccc7ccccc7c6)ccc6oc7ccccc7c56)cc4)nc(-c4cccc5oc6ccccc6c45)n3)ccc2c1]

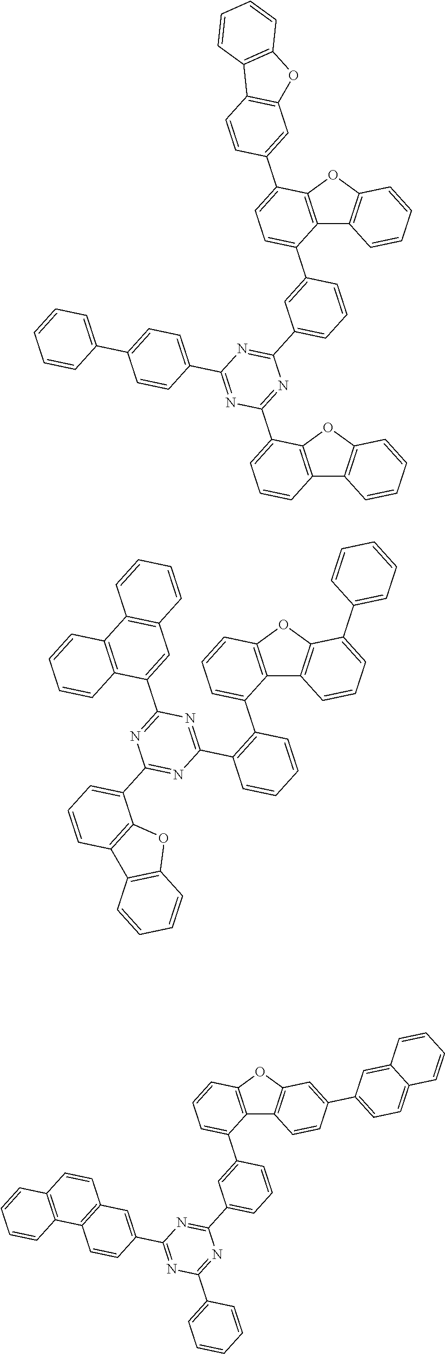

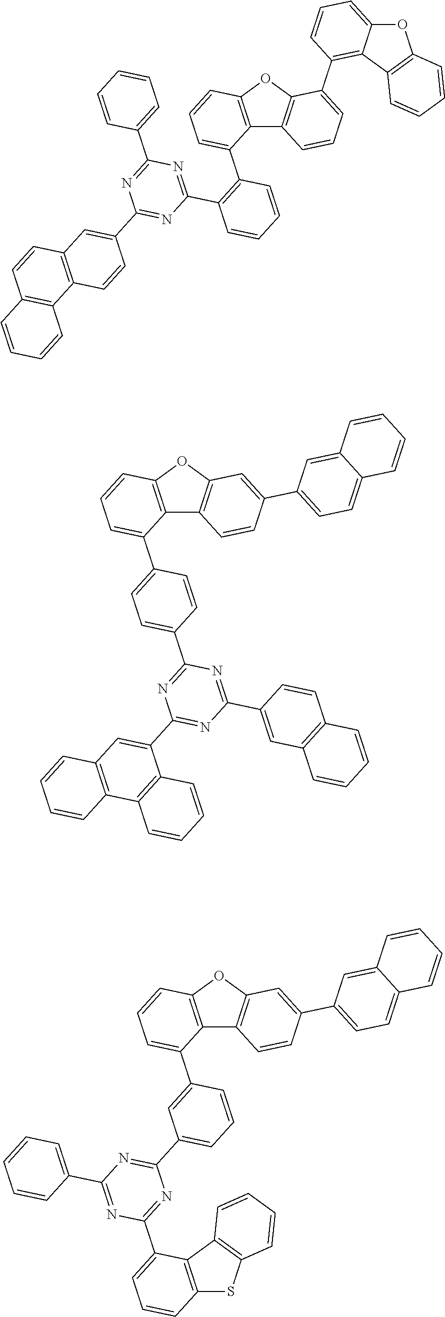 [SMILES: c1ccc(-c2nc(-c3ccc4c(ccc5ccccc54)c3)nc(-c3ccccc3-c3cccc4oc5c(-c6cccc7oc8ccccc8c67)cccc5c34)n2)cc1 ; c1ccc2cc(-c3ccc4c(c3)oc3cccc(-c5ccc(-c6nc(-c7ccc8ccccc8c7)nc(-c7cc8ccccc8c8ccccc78)n6)cc5)c34)ccc2c1 ; c1ccc(-c2nc(-c3cccc(-c4cccc5oc6cc(-c7ccc8ccccc8c7)ccc6c45)c3)nc(-c3cccc4sc5ccccc5c34)n2)cc1]

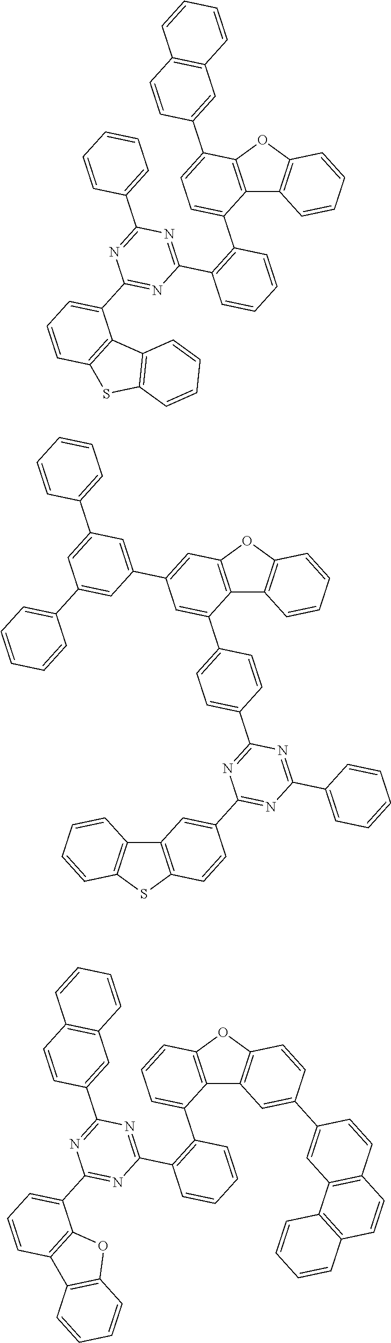 [SMILES: c1ccc(-c2nc(-c3ccccc3-c3ccc(-c4ccc5ccccc5c4)c4oc5ccccc5c34)nc(-c3cccc4sc5ccccc5c34)n2)cc1 ; c1ccc(-c2cc(-c3ccccc3)cc(-c3cc(-c4ccc(-c5nc(-c6ccccc6)nc(-c6ccc7sc8ccccc8c7c6)n5)cc4)c4c(c3)oc3ccccc34)c2)cc1 ; c1ccc(-c2cccc3oc4ccc(-c5ccc6ccc7ccccc7c6c5)cc4c23)c(-c2nc(-c3ccc4ccccc4c3)nc(-c3cccc4c3oc3ccccc34)n2)c1]

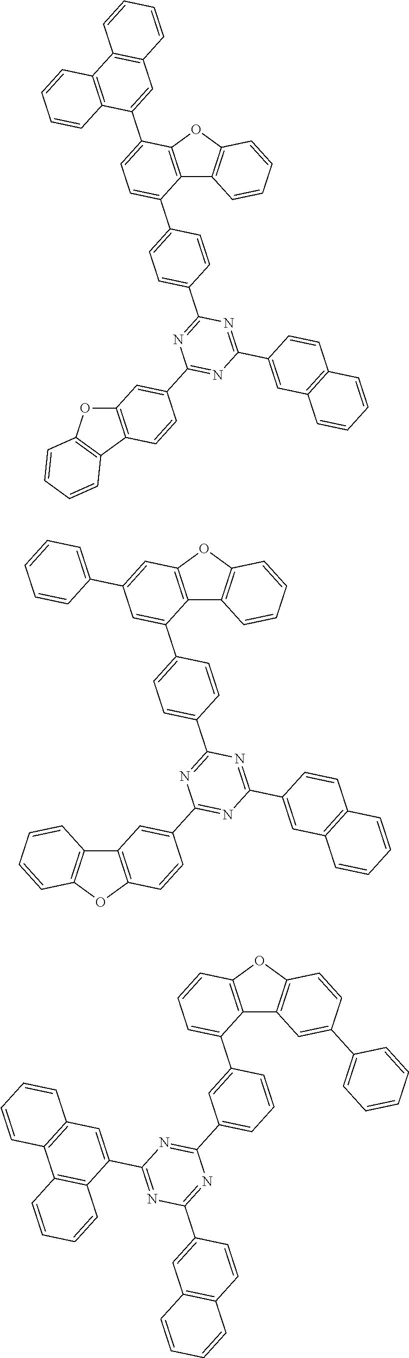 [SMILES: c1ccc2cc(-c3nc(-c4ccc(-c5ccc(-c6cc7ccccc7c7ccccc67)c6oc7ccccc7c56)cc4)nc(-c4ccc5c(c4)oc4ccccc45)n3)ccc2c1 ; c1ccc(-c2cc(-c3ccc(-c4nc(-c5ccc6ccccc6c5)nc(-c5ccc6oc7ccccc7c6c5)n4)cc3)c3c(c2)oc2ccccc23)cc1 ; c1ccc(-c2ccc3oc4cccc(-c5cccc(-c6nc(-c7ccc8ccccc8c7)nc(-c7cc8ccccc8c8ccccc78)n6)c5)c4c3c2)cc1]

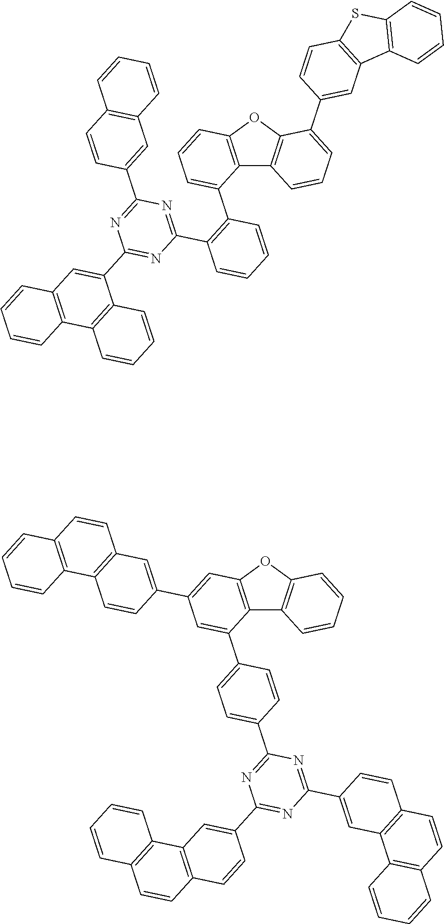 [SMILES: c1ccc(-c2cccc3oc4c(-c5ccc6sc7ccccc7c6c5)cccc4c23)c(-c2nc(-c3ccc4ccccc4c3)nc(-c3cc4ccccc4c4ccccc34)n2)c1 ; c1ccc2c(c1)ccc1cc(-c3cc(-c4ccc(-c5nc(-c6ccc7ccc8ccccc8c7c6)nc(-c6ccc7ccc8ccccc8c7c6)n5)cc4)c4c(c3)oc3ccccc34)ccc12]

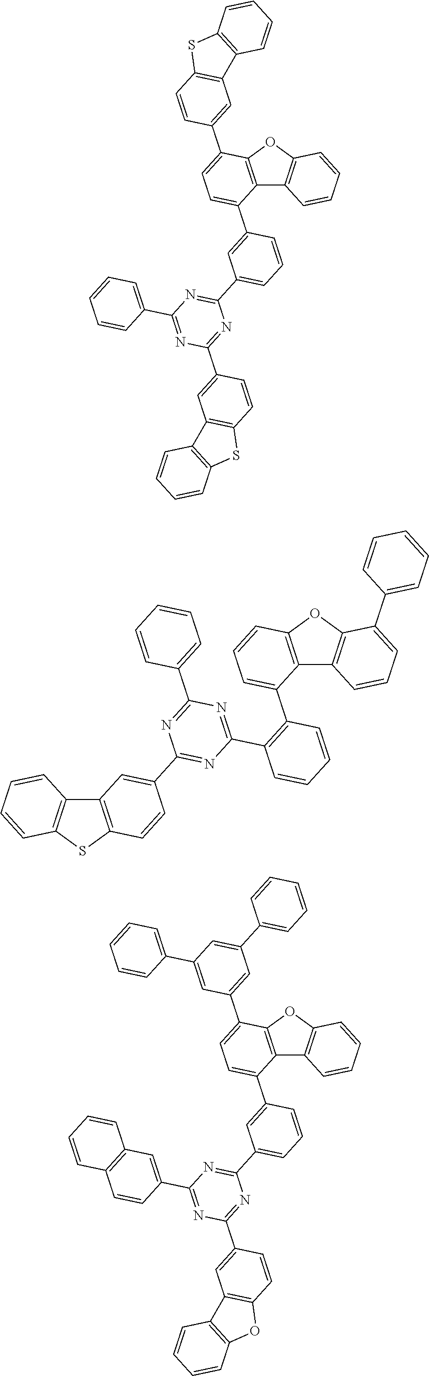 [SMILES: c1ccc(-c2nc(-c3cccc(-c4ccc(-c5ccc6sc7ccccc7c6c5)c5oc6ccccc6c45)c3)nc(-c3ccc4sc5ccccc5c4c3)n2)cc1 ; c1ccc(-c2nc(-c3ccc4sc5ccccc5c4c3)nc(-c3ccccc3-c3cccc4oc5c(-c6ccccc6)cccc5c34)n2)cc1 ; c1ccc(-c2cc(-c3ccccc3)cc(-c3ccc(-c4cccc(-c5nc(-c6ccc7ccccc7c6)nc(-c6ccc7oc8ccccc8c7c6)n5)c4)c4c3oc3ccccc34)c2)cc1]

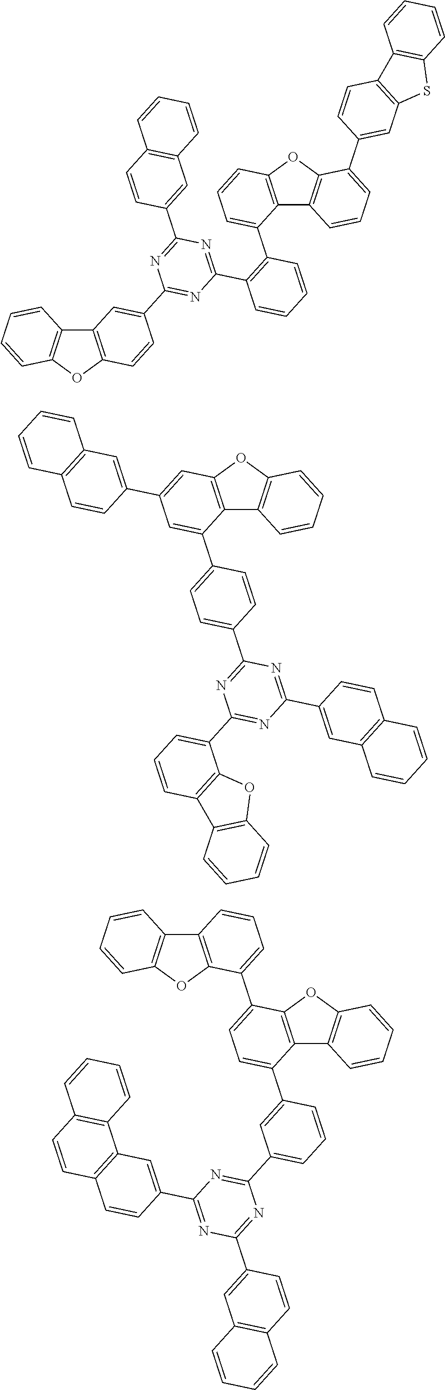

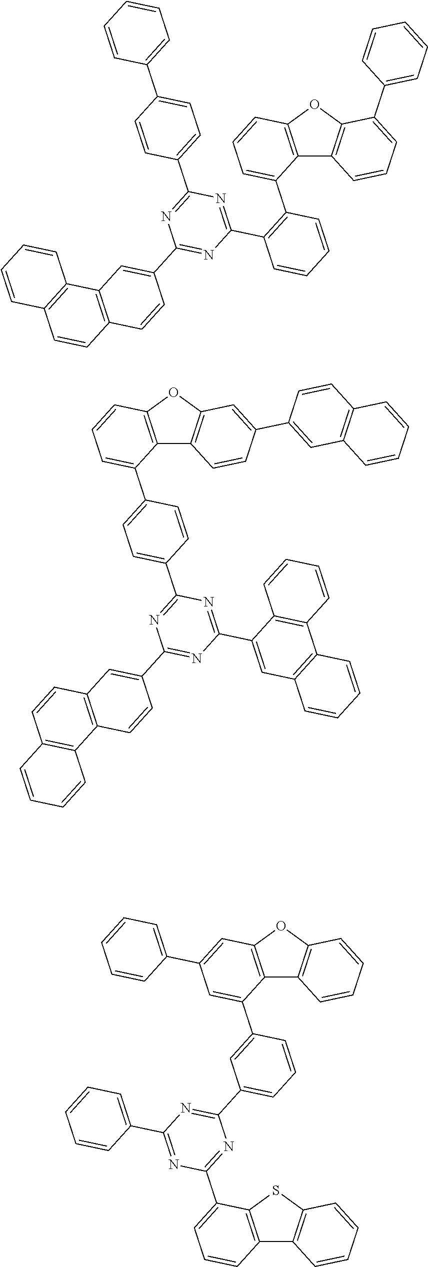 [SMILES: c1ccc(-c2ccc(-c3nc(-c4ccc5ccc6ccccc6c5c4)nc(-c4ccccc4-c4cccc5oc6c(-c7ccccc7)cccc6c45)n3)cc2)cc1 ; c1ccc2cc(-c3ccc4c(c3)oc3cccc(-c5ccc(-c6nc(-c7ccc8c(ccc9ccccc98)c7)nc(-c7cc8ccccc8c8ccccc78)n6)cc5)c34)ccc2c1 ; c1ccc(-c2cc(-c3cccc(-c4nc(-c5ccccc5)nc(-c5cccc6c5sc5ccccc56)n4)c3)c3c(c2)oc2ccccc23)cc1]

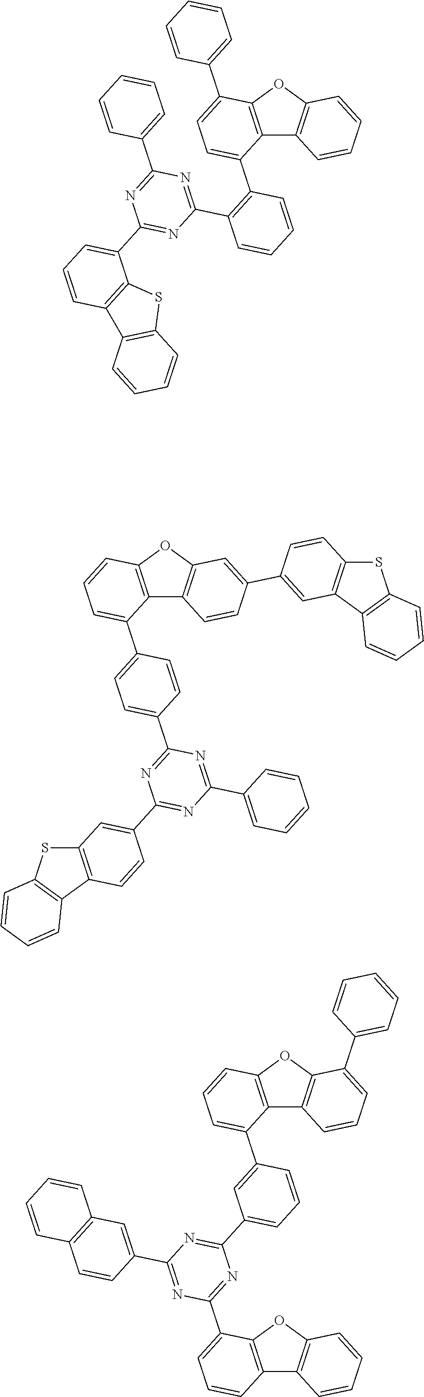 [SMILES: c1ccc(-c2nc(-c3ccccc3-c3ccc(-c4ccccc4)c4oc5ccccc5c34)nc(-c3cccc4c3sc3ccccc34)n2)cc1 ; c1ccc(-c2nc(-c3ccc(-c4cccc5oc6cc(-c7ccc8sc9ccccc9c8c7)ccc6c45)cc3)nc(-c3ccc4c(c3)sc3ccccc34)n2)cc1 ; c1ccc(-c2cccc3c2oc2cccc(-c4cccc(-c5nc(-c6ccc7ccccc7c6)nc(-c6cccc7c6oc6ccccc67)n5)c4)c23)cc1]

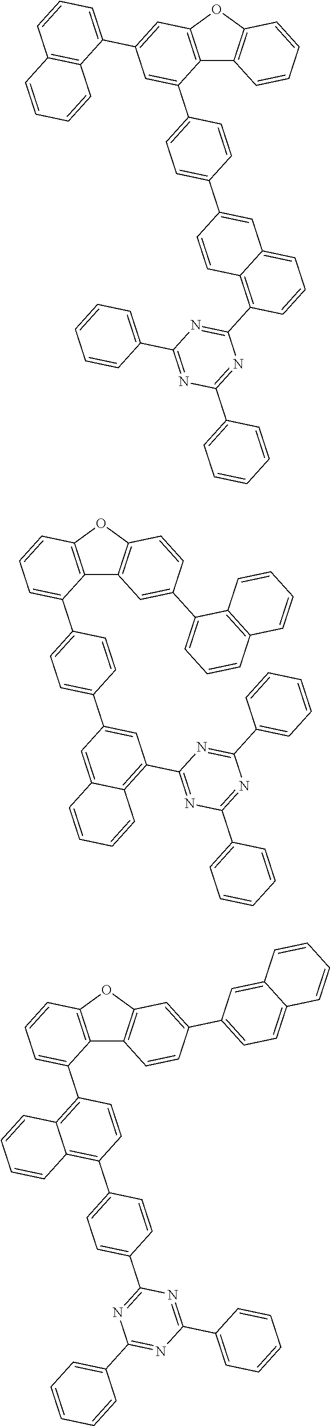 [SMILES: c1ccc(-c2nc(-c3ccccc3)nc(-c3cccc4cc(-c5ccc(-c6cc(-c7cccc8ccccc78)cc7oc8ccccc8c67)cc5)ccc34)n2)cc1 ; c1ccc(-c2nc(-c3ccccc3)nc(-c3cc(-c4ccc(-c5cccc6oc7ccc(-c8cccc9ccccc89)cc7c56)cc4)cc4ccccc34)n2)cc1 ; c1ccc(-c2nc(-c3ccccc3)nc(-c3ccc(-c4ccc(-c5cccc6oc7cc(-c8ccc9ccccc9c8)ccc7c56)c5ccccc45)cc3)n2)cc1]

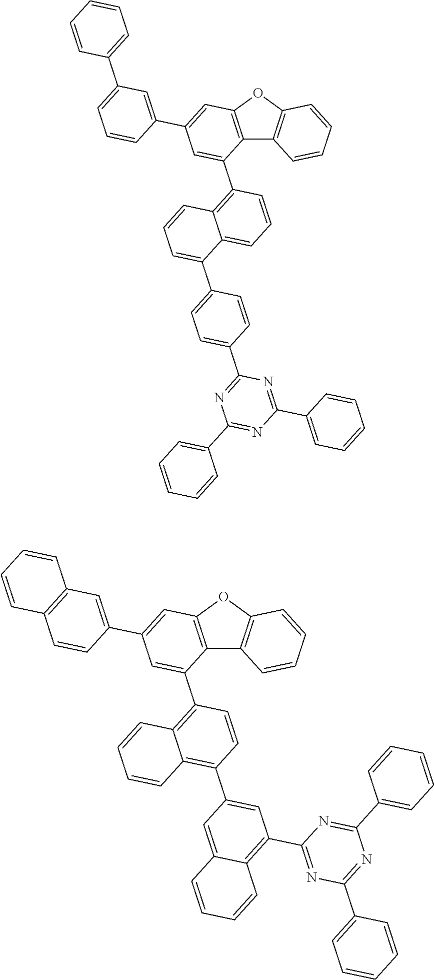 [SMILES: c1ccc(-c2cccc(-c3cc(-c4cccc5c(-c6ccc(-c7nc(-c8ccccc8)nc(-c8ccccc8)n7)cc6)cccc45)c4c(c3)oc3ccccc34)c2)cc1 ; c1ccc(-c2nc(-c3ccccc3)nc(-c3cc(-c4ccc(-c5cc(-c6ccc7ccccc7c6)cc6oc7ccccc7c56)c5ccccc45)cc4ccccc34)n2)cc1]

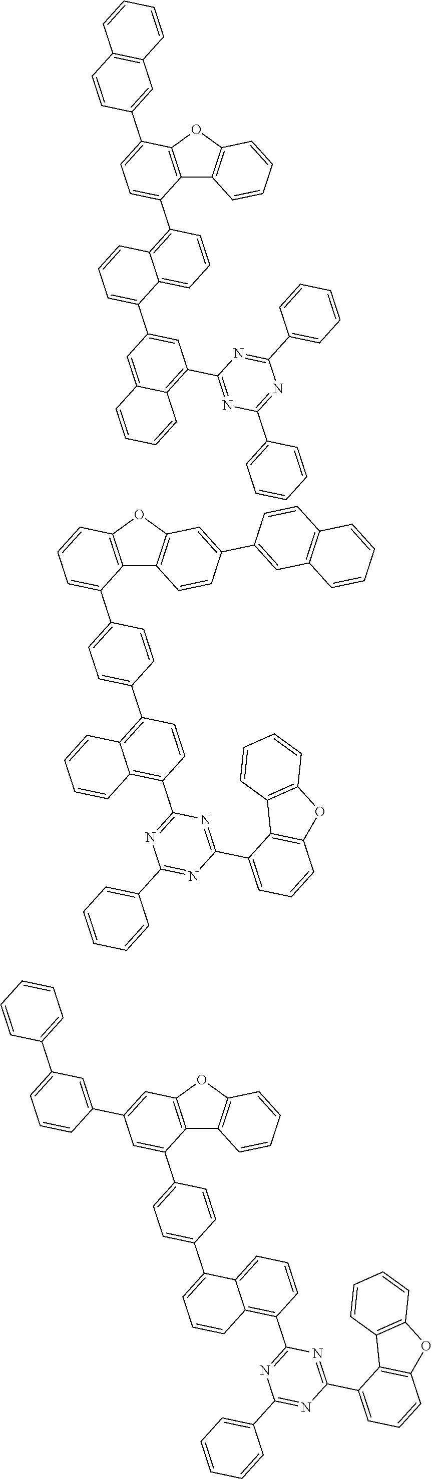

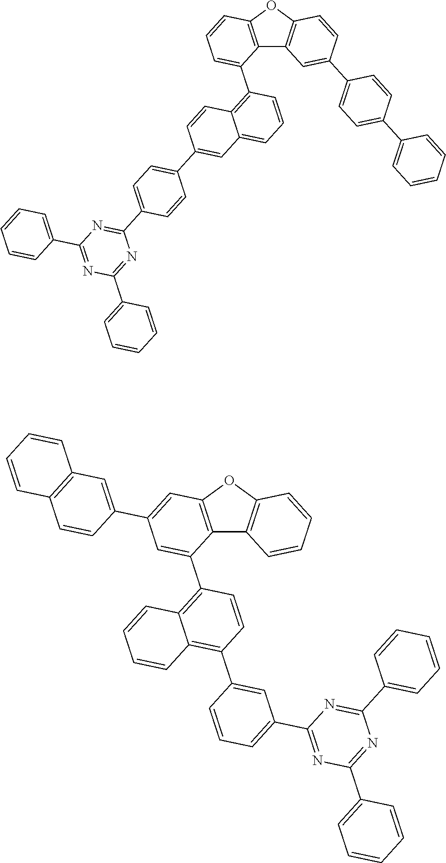 [SMILES: c1ccc(-c2ccc(-c3ccc4oc5cccc(-c6cccc7cc(-c8ccc(-c9nc(-c%10ccccc%10)nc(-c%10ccccc%10)n9)cc8)ccc67)c5c4c3)cc2)cc1 ; c1ccc(-c2nc(-c3ccccc3)nc(-c3cccc(-c4ccc(-c5cc(-c6ccc7ccccc7c6)cc6oc7ccccc7c56)c5ccccc45)c3)n2)cc1]

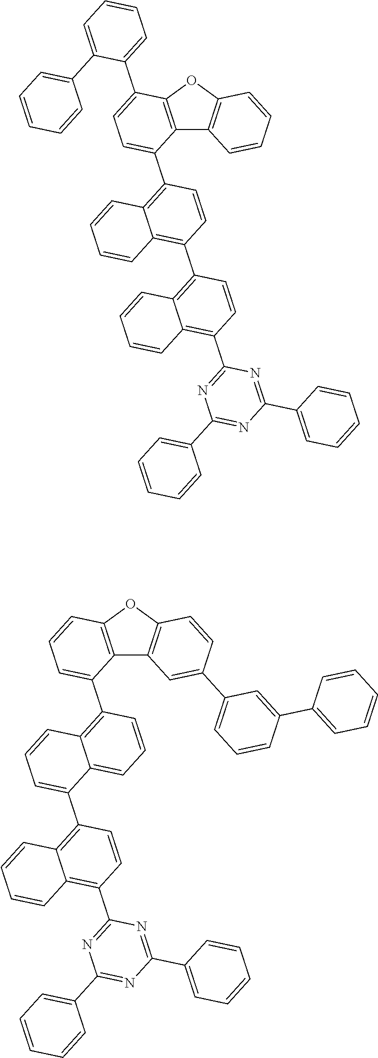 [SMILES: c1ccc(-c2nc(-c3ccccc3)nc(-c3ccc(-c4ccc(-c5ccc(-c6ccccc6-c6ccccc6)c6oc7ccccc7c56)c5ccccc45)c4ccccc34)n2)cc1 ; c1ccc(-c2cccc(-c3ccc4oc5cccc(-c6cccc7c(-c8ccc(-c9nc(-c%10ccccc%10)nc(-c%10ccccc%10)n9)c9ccccc89)cccc67)c5c4c3)c2)cc1]

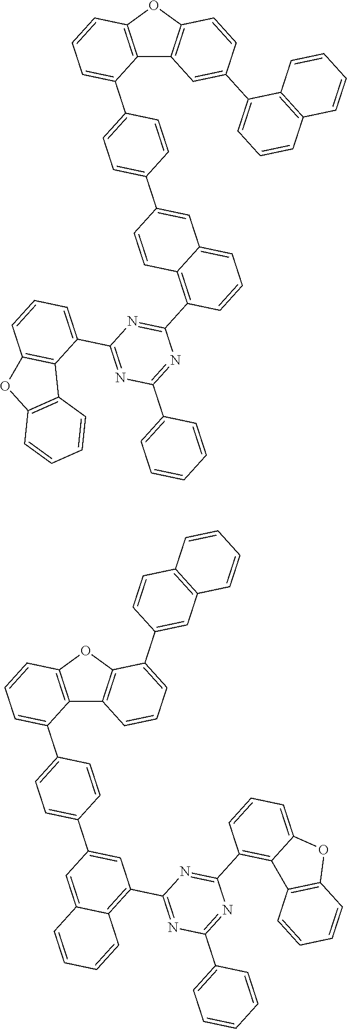 [SMILES: c1ccc(-c2nc(-c3cccc4cc(-c5ccc(-c6cccc7oc8ccc(-c9cccc%10ccccc9%10)cc8c67)cc5)ccc34)nc(-c3cccc4oc5ccccc5c34)n2)cc1 ; c1ccc(-c2nc(-c3cc(-c4ccc(-c5cccc6oc7c(-c8ccc9ccccc9c8)cccc7c56)cc4)cc4ccccc34)nc(-c3cccc4oc5ccccc5c34)n2)cc1]

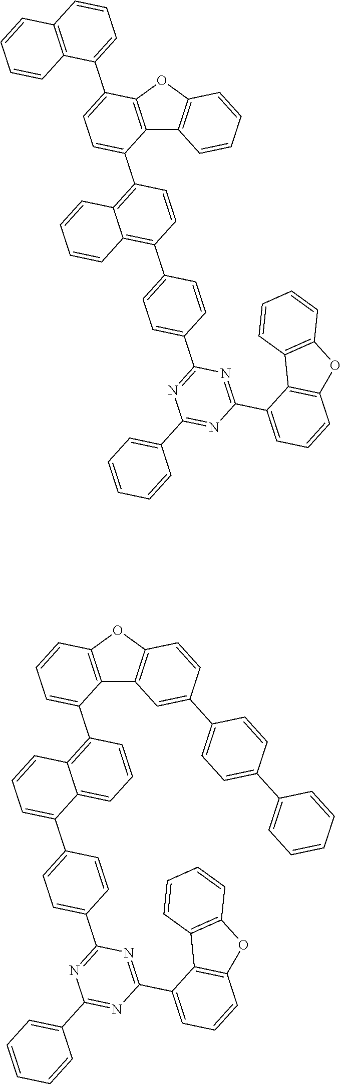 [SMILES: c1ccc(-c2nc(-c3ccc(-c4ccc(-c5ccc(-c6cccc7ccccc67)c6oc7ccccc7c56)c5ccccc45)cc3)nc(-c3cccc4oc5ccccc5c34)n2)cc1 ; c1ccc(-c2ccc(-c3ccc4oc5cccc(-c6cccc7c(-c8ccc(-c9nc(-c%10ccccc%10)nc(-c%10cccc%11oc%12ccccc%12c%10%11)n9)cc8)cccc67)c5c4c3)cc2)cc1]

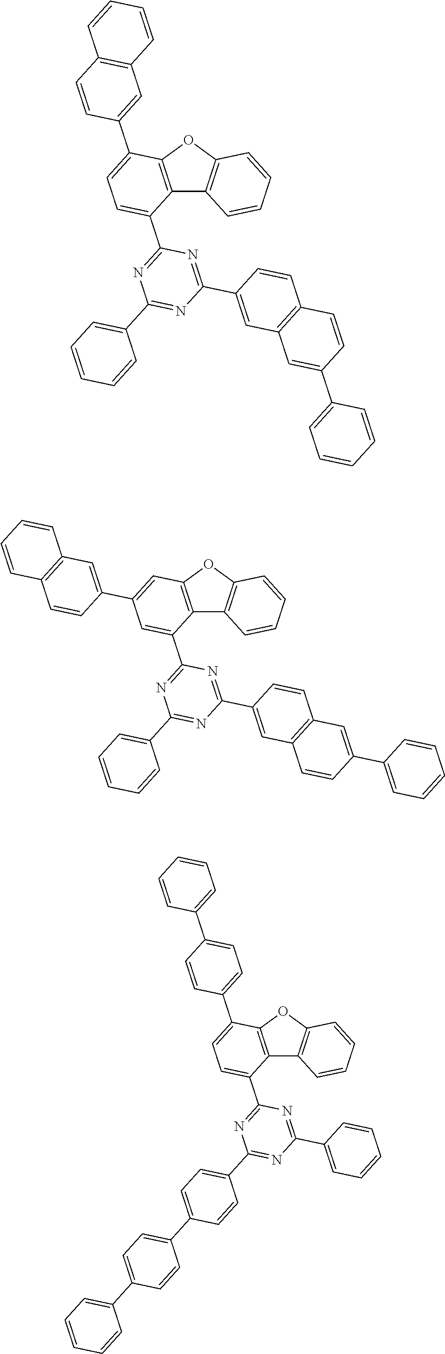 [SMILES: c1ccc(-c2ccc3ccc(-c4nc(-c5ccccc5)nc(-c5ccc(-c6ccc7ccccc7c6)c6oc7ccccc7c56)n4)cc3c2)cc1 ; c1ccc(-c2ccc3cc(-c4nc(-c5ccccc5)nc(-c5cc(-c6ccc7ccccc7c6)cc6oc7ccccc7c56)n4)ccc3c2)cc1 ; c1ccc(-c2ccc(-c3ccc(-c4nc(-c5ccccc5)nc(-c5ccc(-c6ccc(-c7ccccc7)cc6)c6oc7ccccc7c56)n4)cc3)cc2)cc1]

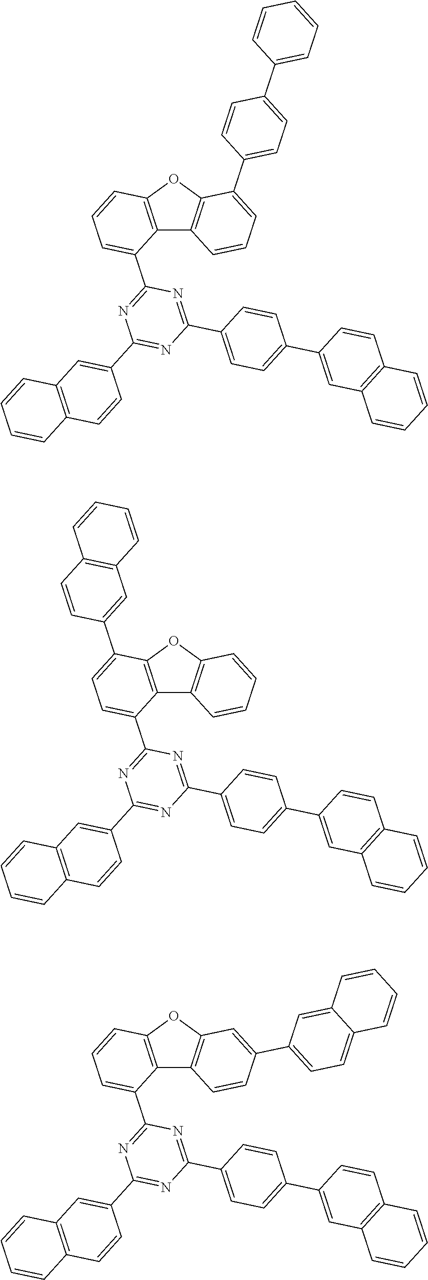 [SMILES: c1ccc(-c2ccc(-c3cccc4c3oc3cccc(-c5nc(-c6ccc(-c7ccc8ccccc8c7)cc6)nc(-c6ccc7ccccc7c6)n5)c34)cc2)cc1 ; c1ccc2cc(-c3ccc(-c4nc(-c5ccc6ccccc6c5)nc(-c5ccc(-c6ccc7ccccc7c6)c6oc7ccccc7c56)n4)cc3)ccc2c1 ; c1ccc2cc(-c3ccc(-c4nc(-c5ccc6ccccc6c5)nc(-c5cccc6oc7cc(-c8ccc9ccccc9c8)ccc7c56)n4)cc3)ccc2c1]

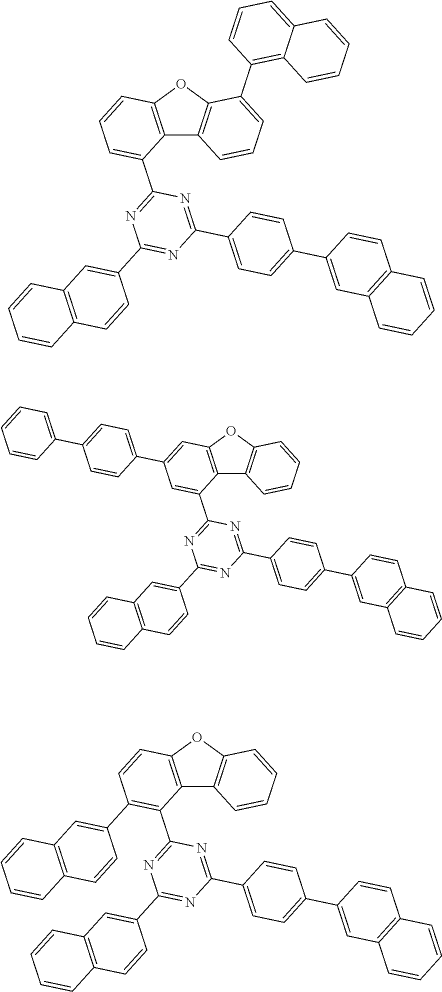 [SMILES: c1ccc2cc(-c3ccc(-c4nc(-c5ccc6ccccc6c5)nc(-c5cccc6oc7c(-c8cccc9ccccc89)cccc7c56)n4)cc3)ccc2c1 ; c1ccc(-c2ccc(-c3cc(-c4nc(-c5ccc(-c6ccc7ccccc7c6)cc5)nc(-c5ccc6ccccc6c5)n4)c4c(c3)oc3ccccc34)cc2)cc1 ; c1ccc2cc(-c3ccc(-c4nc(-c5ccc6ccccc6c5)nc(-c5c(-c6ccc7ccccc7c6)ccc6oc7ccccc7c56)n4)cc3)ccc2c1]

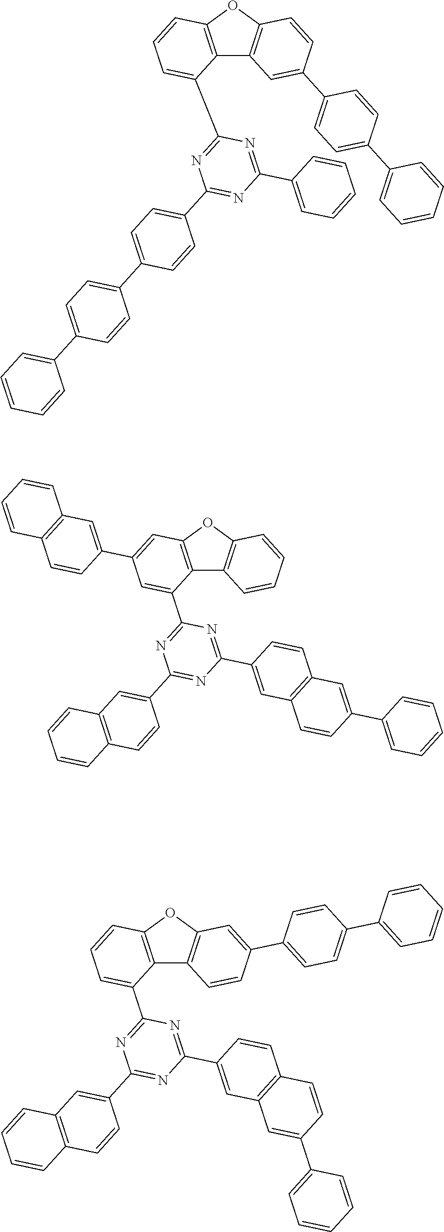 [SMILES: c1ccc(-c2ccc(-c3ccc(-c4nc(-c5ccccc5)nc(-c5cccc6oc7ccc(-c8ccc(-c9ccccc9)cc8)cc7c56)n4)cc3)cc2)cc1 ; c1ccc(-c2ccc3cc(-c4nc(-c5ccc6ccccc6c5)nc(-c5cc(-c6ccc7ccccc7c6)cc6oc7ccccc7c56)n4)ccc3c2)cc1 ; c1ccc(-c2ccc(-c3ccc4c(c3)oc3cccc(-c5nc(-c6ccc7ccccc7c6)nc(-c6ccc7ccc(-c8ccccc8)cc7c6)n5)c34)cc2)cc1]

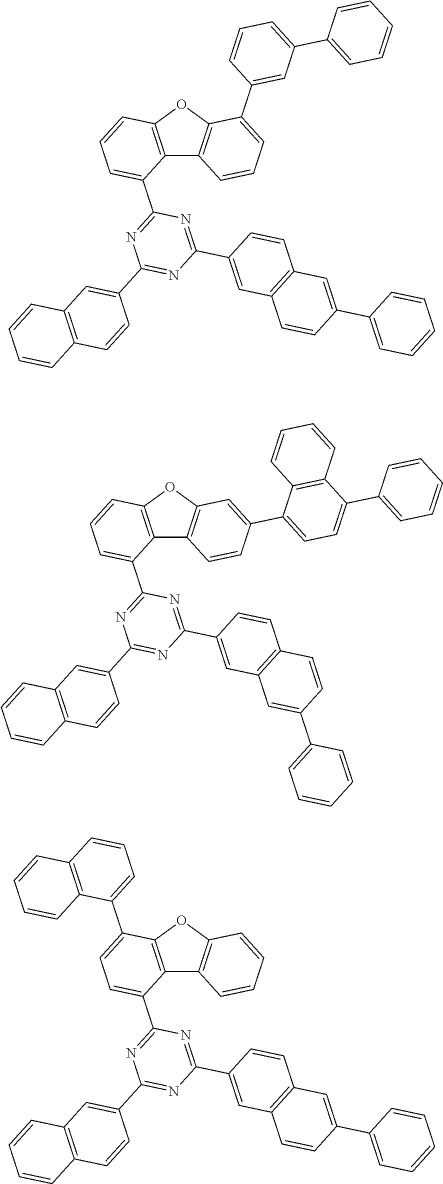 [SMILES: c1ccc(-c2cccc(-c3cccc4c3oc3cccc(-c5nc(-c6ccc7ccccc7c6)nc(-c6ccc7cc(-c8ccccc8)ccc7c6)n5)c34)c2)cc1 ; c1ccc(-c2ccc3ccc(-c4nc(-c5ccc6ccccc6c5)nc(-c5cccc6oc7cc(-c8ccc(-c9ccccc9)c9ccccc89)ccc7c56)n4)cc3c2)cc1 ; c1ccc(-c2ccc3cc(-c4nc(-c5ccc6ccccc6c5)nc(-c5ccc(-c6cccc7ccccc67)c6oc7ccccc7c56)n4)ccc3c2)cc1]

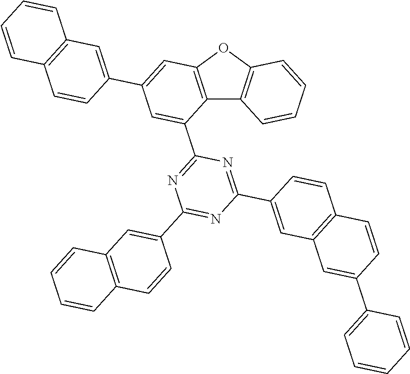 [SMILES: c1ccc(-c2ccc3ccc(-c4nc(-c5ccc6ccccc6c5)nc(-c5cc(-c6ccc7ccccc7c6)cc6oc7ccccc7c56)n4)cc3c2)cc1]

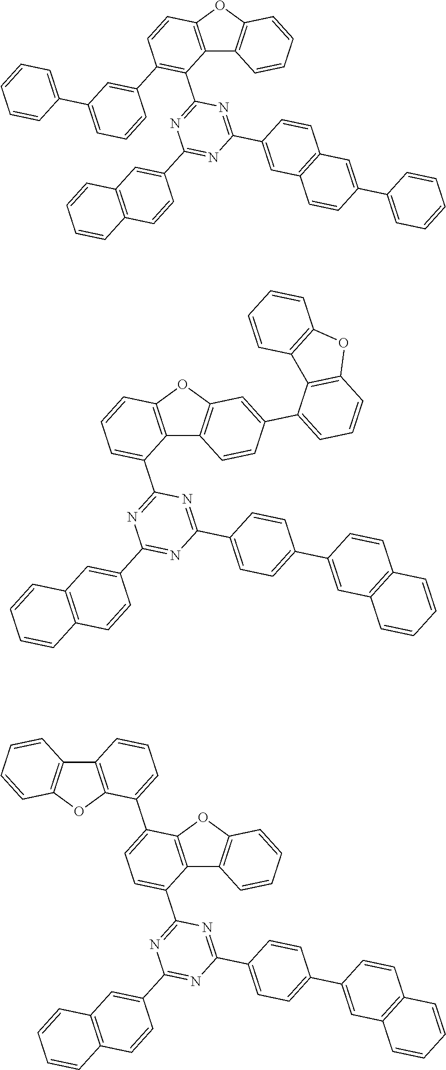 [SMILES: c1ccc(-c2cccc(-c3ccc4oc5ccccc5c4c3-c3nc(-c4ccc5ccccc5c4)nc(-c4ccc5cc(-c6ccccc6)ccc5c4)n3)c2)cc1 ; c1ccc2cc(-c3ccc(-c4nc(-c5ccc6ccccc6c5)nc(-c5cccc6oc7cc(-c8cccc9oc%10ccccc%10c89)ccc7c56)n4)cc3)ccc2c1 ; c1ccc2cc(-c3ccc(-c4nc(-c5ccc6ccccc6c5)nc(-c5ccc(-c6cccc7c6oc6ccccc67)c6oc7ccccc7c56)n4)cc3)ccc2c1]

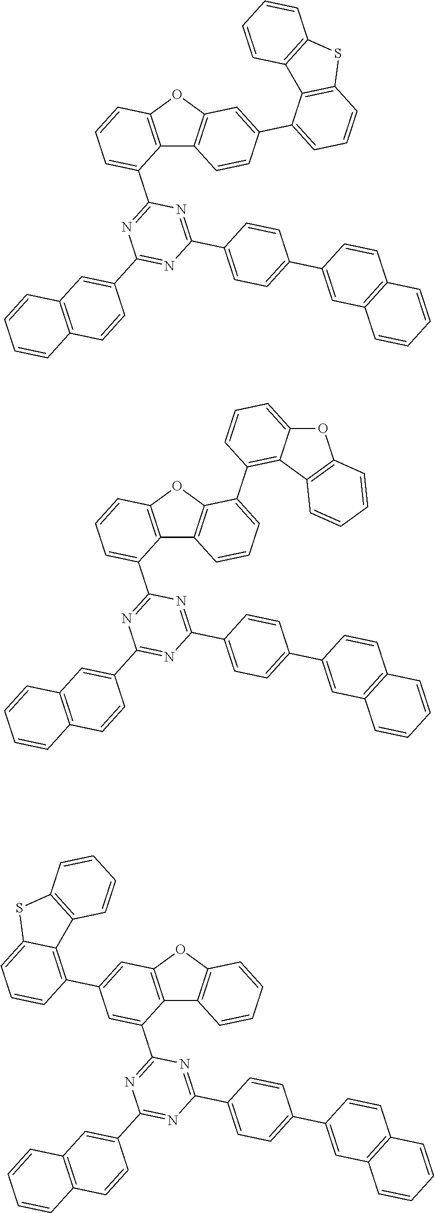 [SMILES: c1ccc2cc(-c3ccc(-c4nc(-c5ccc6ccccc6c5)nc(-c5cccc6oc7cc(-c8cccc9sc%10ccccc%10c89)ccc7c56)n4)cc3)ccc2c1 ; c1ccc2cc(-c3ccc(-c4nc(-c5ccc6ccccc6c5)nc(-c5cccc6oc7c(-c8cccc9oc%10ccccc%10c89)cccc7c56)n4)cc3)ccc2c1 ; c1ccc2cc(-c3ccc(-c4nc(-c5ccc6ccccc6c5)nc(-c5cc(-c6cccc7sc8ccccc8c67)cc6oc7ccccc7c56)n4)cc3)ccc2c1]

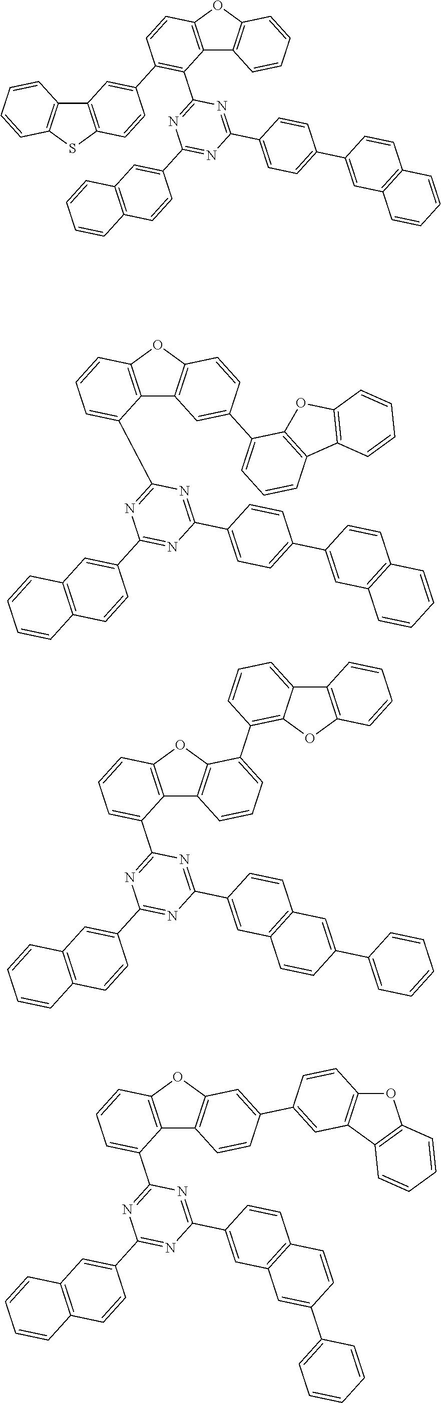 [SMILES: c1ccc2cc(-c3ccc(-c4nc(-c5ccc6ccccc6c5)nc(-c5c(-c6ccc7sc8ccccc8c7c6)ccc6oc7ccccc7c56)n4)cc3)ccc2c1 ; c1ccc2cc(-c3ccc(-c4nc(-c5ccc6ccccc6c5)nc(-c5cccc6oc7ccc(-c8cccc9c8oc8ccccc89)cc7c56)n4)cc3)ccc2c1 ; c1ccc(-c2ccc3cc(-c4nc(-c5ccc6ccccc6c5)nc(-c5cccc6oc7c(-c8cccc9c8oc8ccccc89)cccc7c56)n4)ccc3c2)cc1 ; c1ccc(-c2ccc3ccc(-c4nc(-c5ccc6ccccc6c5)nc(-c5cccc6oc7cc(-c8ccc9oc%10ccccc%10c9c8)ccc7c56)n4)cc3c2)cc1]

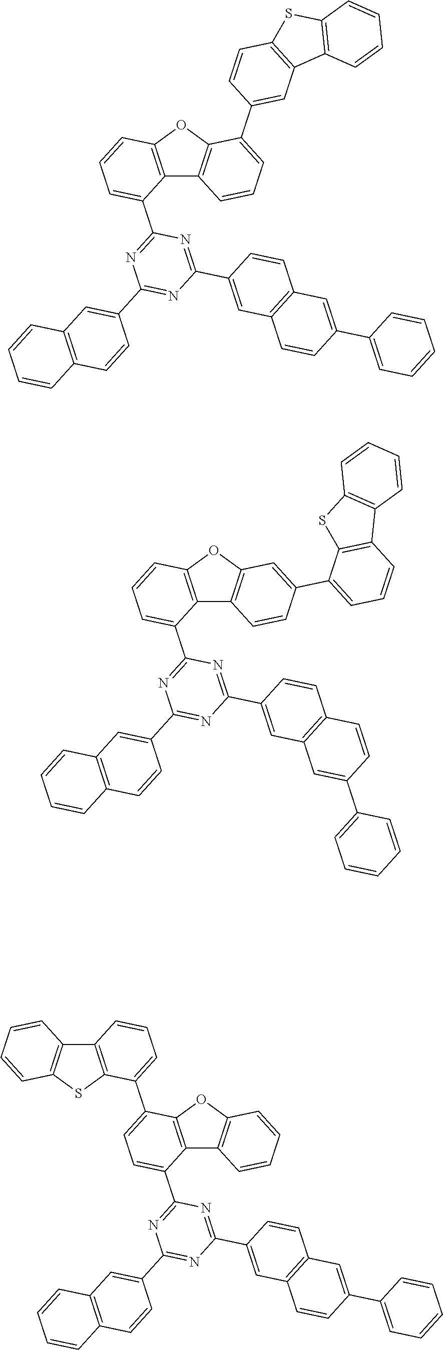 [SMILES: c1ccc(-c2ccc3cc(-c4nc(-c5ccc6ccccc6c5)nc(-c5cccc6oc7c(-c8ccc9sc%10ccccc%10c9c8)cccc7c56)n4)ccc3c2)cc1 ; c1ccc(-c2ccc3ccc(-c4nc(-c5ccc6ccccc6c5)nc(-c5cccc6oc7cc(-c8cccc9c8sc8ccccc89)ccc7c56)n4)cc3c2)cc1 ; c1ccc(-c2ccc3cc(-c4nc(-c5ccc6ccccc6c5)nc(-c5ccc(-c6cccc7c6sc6ccccc67)c6oc7ccccc7c56)n4)ccc3c2)cc1]

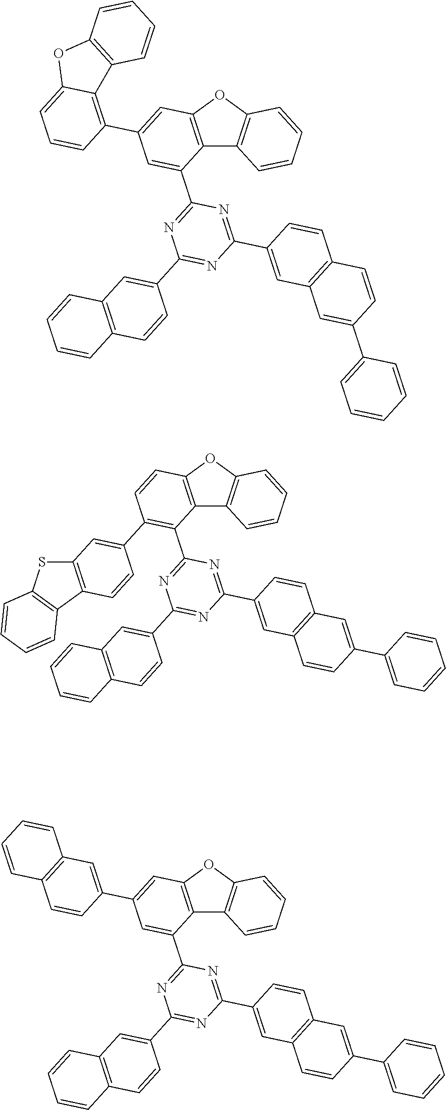 [SMILES: c1ccc(-c2ccc3ccc(-c4nc(-c5ccc6ccccc6c5)nc(-c5cc(-c6cccc7oc8ccccc8c67)cc6oc7ccccc7c56)n4)cc3c2)cc1 ; c1ccc(-c2ccc3cc(-c4nc(-c5ccc6ccccc6c5)nc(-c5c(-c6ccc7c(c6)sc6ccccc67)ccc6oc7ccccc7c56)n4)ccc3c2)cc1 ; c1ccc(-c2ccc3cc(-c4nc(-c5ccc6ccccc6c5)nc(-c5cc(-c6ccc7ccccc7c6)cc6oc7ccccc7c56)n4)ccc3c2)cc1]

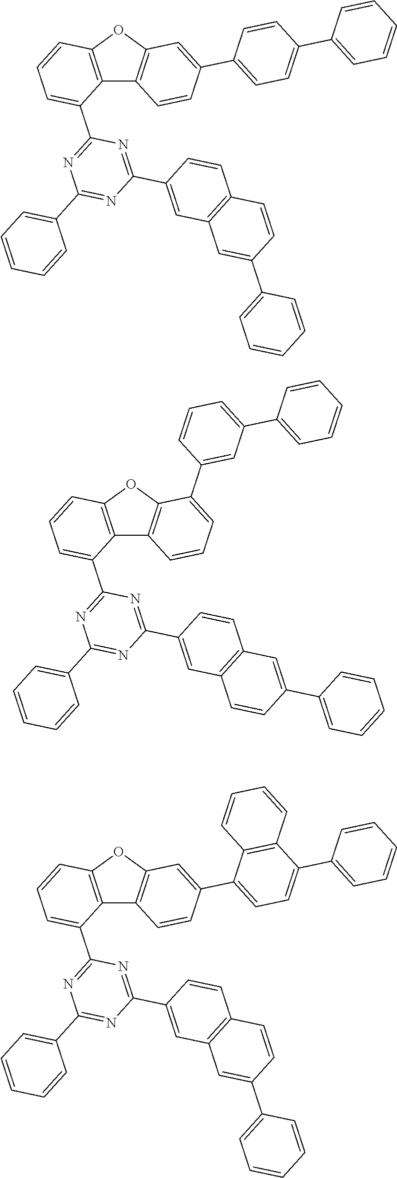

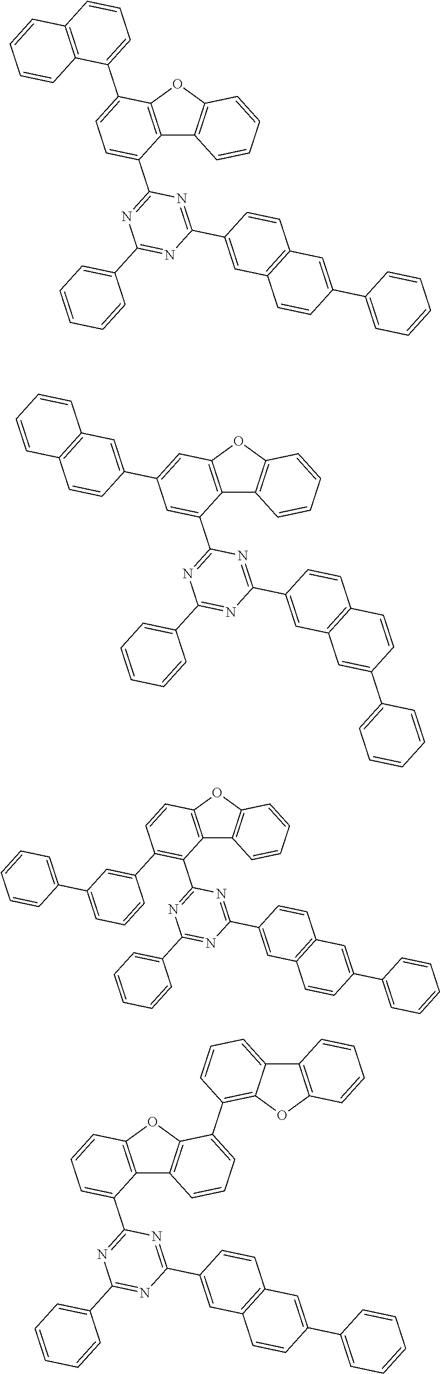

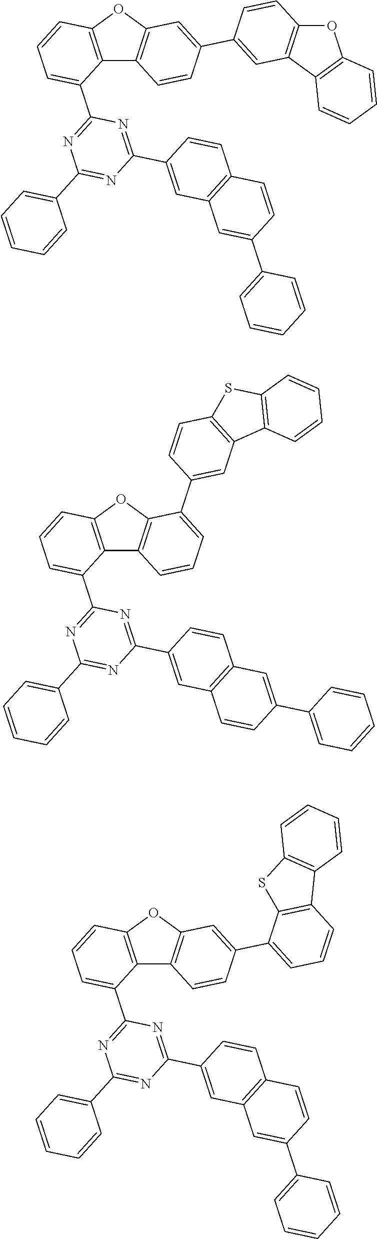

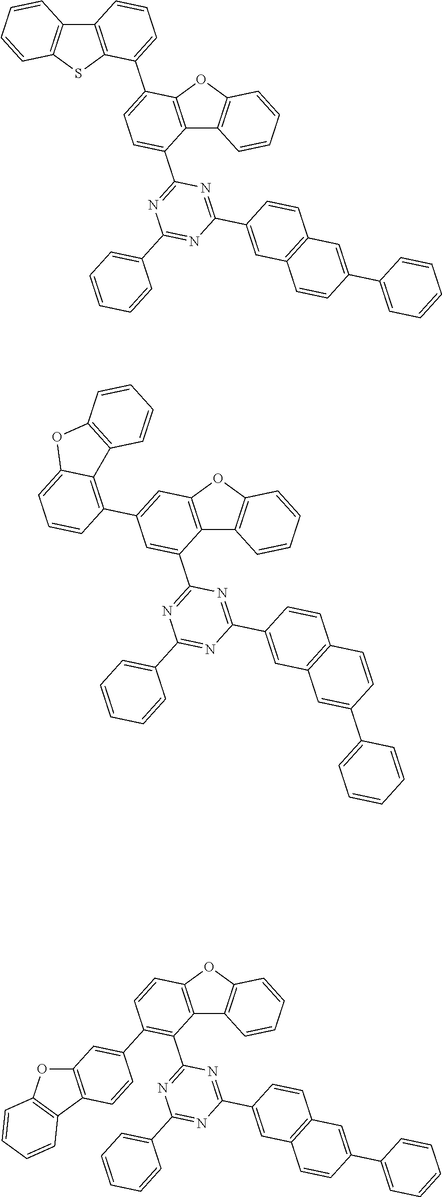 [SMILES: c1ccc(-c2ccc3cc(-c4nc(-c5ccccc5)nc(-c5ccc(-c6cccc7c6sc6ccccc67)c6oc7ccccc7c56)n4)ccc3c2)cc1 ; c1ccc(-c2ccc3ccc(-c4nc(-c5ccccc5)nc(-c5cc(-c6cccc7oc8ccccc8c67)cc6oc7ccccc7c56)n4)cc3c2)cc1 ; c1ccc(-c2ccc3cc(-c4nc(-c5ccccc5)nc(-c5c(-c6ccc7c(c6)oc6ccccc67)ccc6oc7ccccc7c56)n4)ccc3c2)cc1]

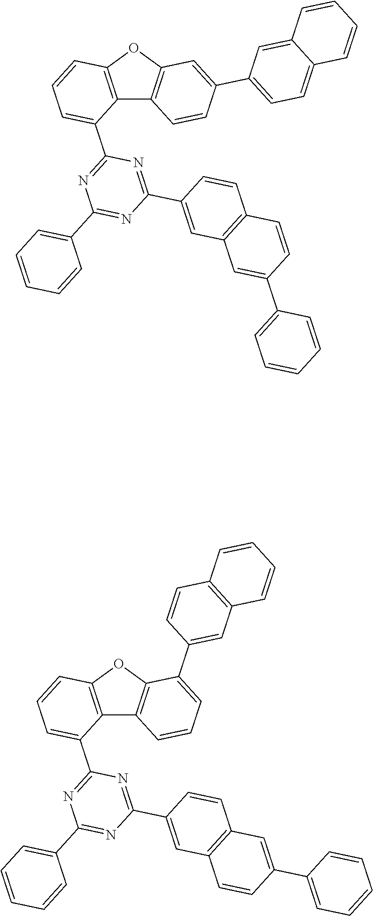 [SMILES: c1ccc(-c2ccc3ccc(-c4nc(-c5ccccc5)nc(-c5cccc6oc7cc(-c8ccc9ccccc9c8)ccc7c56)n4)cc3c2)cc1 ; c1ccc(-c2ccc3cc(-c4nc(-c5ccccc5)nc(-c5cccc6oc7c(-c8ccc9ccccc9c8)cccc7c56)n4)ccc3c2)cc1]

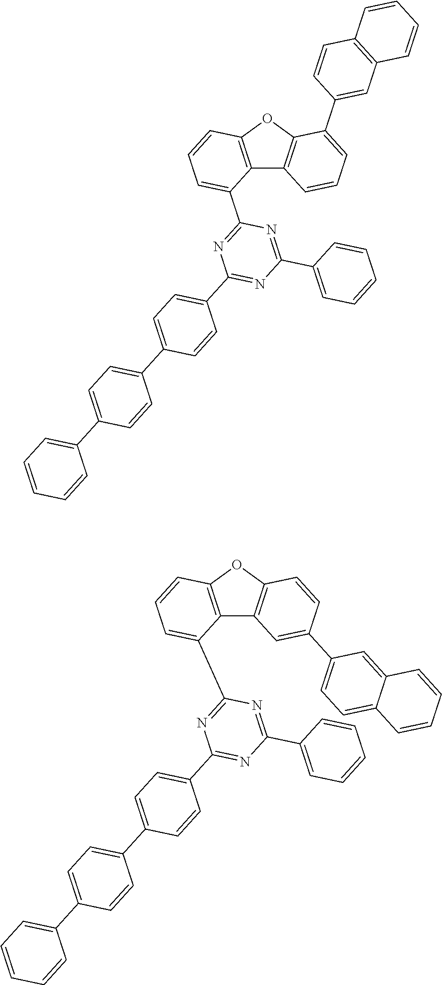 [SMILES: c1ccc(-c2ccc(-c3ccc(-c4nc(-c5ccccc5)nc(-c5cccc6oc7c(-c8ccc9ccccc9c8)cccc7c56)n4)cc3)cc2)cc1 ; c1ccc(-c2ccc(-c3ccc(-c4nc(-c5ccccc5)nc(-c5cccc6oc7ccc(-c8ccc9ccccc9c8)cc7c56)n4)cc3)cc2)cc1]

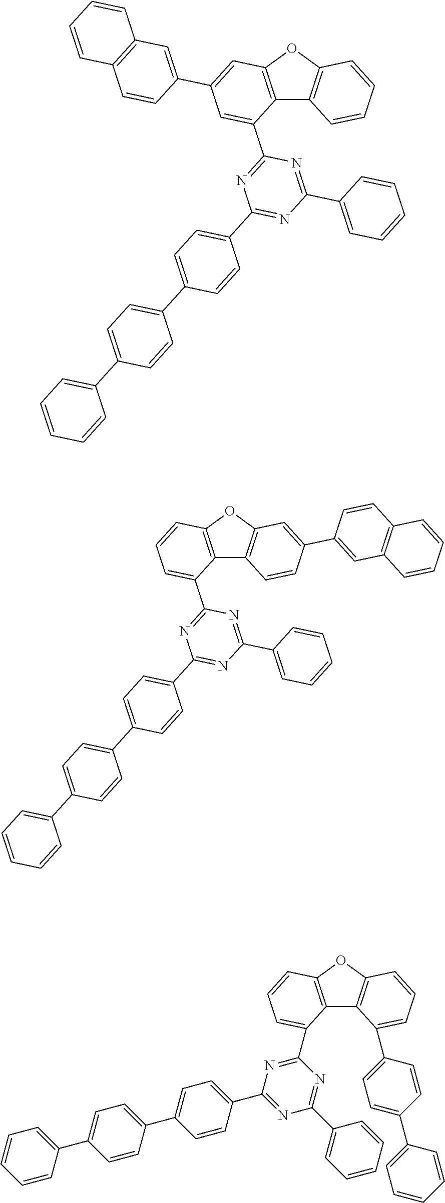 [SMILES: c1ccc(-c2ccc(-c3ccc(-c4nc(-c5ccccc5)nc(-c5cc(-c6ccc7ccccc7c6)cc6oc7ccccc7c56)n4)cc3)cc2)cc1 ; c1ccc(-c2ccc(-c3ccc(-c4nc(-c5ccccc5)nc(-c5cccc6oc7cc(-c8ccc9ccccc9c8)ccc7c56)n4)cc3)cc2)cc1 ; c1ccc(-c2ccc(-c3ccc(-c4nc(-c5ccccc5)nc(-c5cccc6oc7cccc(-c8ccc(-c9ccccc9)cc8)c7c56)n4)cc3)cc2)cc1]

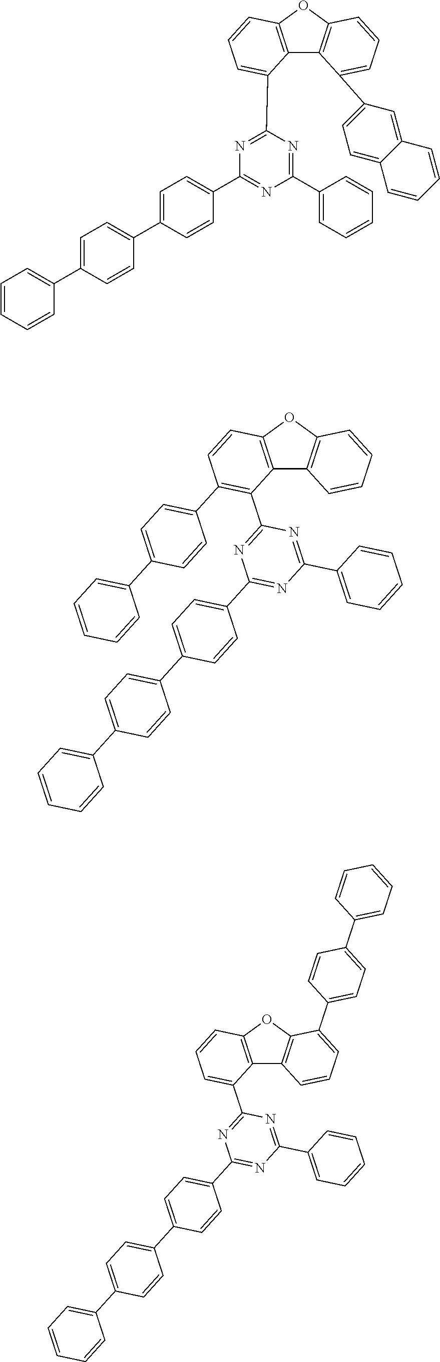 [SMILES: c1ccc(-c2ccc(-c3ccc(-c4nc(-c5ccccc5)nc(-c5cccc6oc7cccc(-c8ccc9ccccc9c8)c7c56)n4)cc3)cc2)cc1 ; c1ccc(-c2ccc(-c3ccc(-c4nc(-c5ccccc5)nc(-c5c(-c6ccc(-c7ccccc7)cc6)ccc6oc7ccccc7c56)n4)cc3)cc2)cc1 ; c1ccc(-c2ccc(-c3ccc(-c4nc(-c5ccccc5)nc(-c5cccc6oc7c(-c8ccc(-c9ccccc9)cc8)cccc7c56)n4)cc3)cc2)cc1]

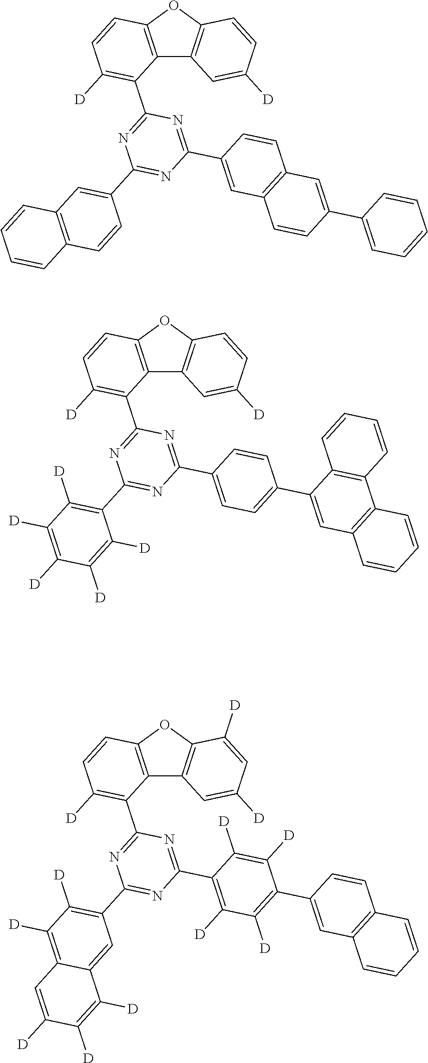 [SMILES: [2H]c1ccc2oc3ccc([2H])c(-c4nc(-c5ccc6ccccc6c5)nc(-c5ccc6cc(-c7ccccc7)ccc6c5)n4)c3c2c1 ; [2H]c1ccc2oc3ccc([2H])c(-c4nc(-c5ccc(-c6cc7ccccc7c7ccccc67)cc5)nc(-c5c([2H])c([2H])c([2H])c([2H])c5[2H])n4)c3c2c1 ; [2H]c1cc([2H])c2oc3ccc([2H])c(-c4nc(-c5cc6c([2H])c([2H])c([2H])cc6c([2H])c5[2H])nc(-c5c([2H])c([2H])c(-c6ccc7ccccc7c6)c([2H])c5[2H])n4)c3c2c1]

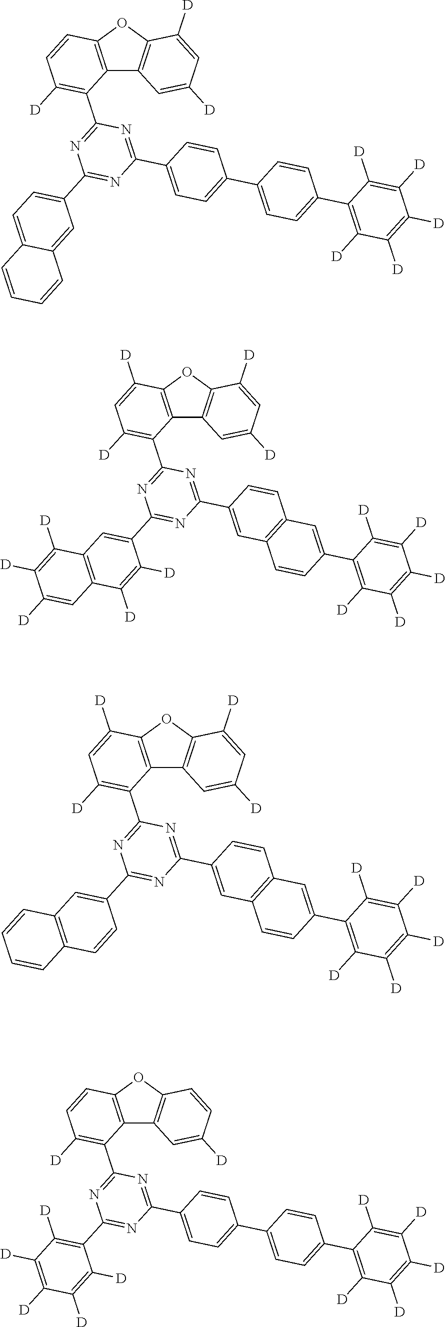

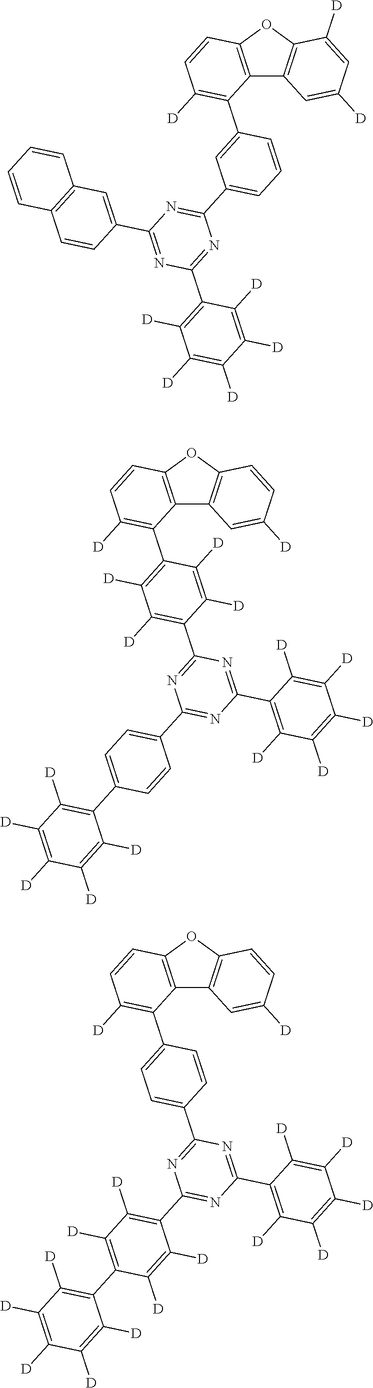 [SMILES: [2H]c1cc([2H])c2oc3ccc([2H])c(-c4cccc(-c5nc(-c6ccc7ccccc7c6)nc(-c6c([2H])c([2H])c([2H])c([2H])c6[2H])n5)c4)c3c2c1 ; [2H]c1ccc2oc3ccc([2H])c(-c4c([2H])c([2H])c(-c5nc(-c6ccc(-c7c([2H])c([2H])c([2H])c([2H])c7[2H])cc6)nc(-c6c([2H])c([2H])c([2H])c([2H])c6[2H])n5)c([2H])c4[2H])c3c2c1 ; [2H]c1ccc2oc3ccc([2H])c(-c4ccc(-c5nc(-c6c([2H])c([2H])c([2H])c([2H])c6[2H])nc(-c6c([2H])c([2H])c(-c7c([2H])c([2H])c([2H])c([2H])c7[2H])c([2H])c6[2H])n5)cc4)c3c2c1]

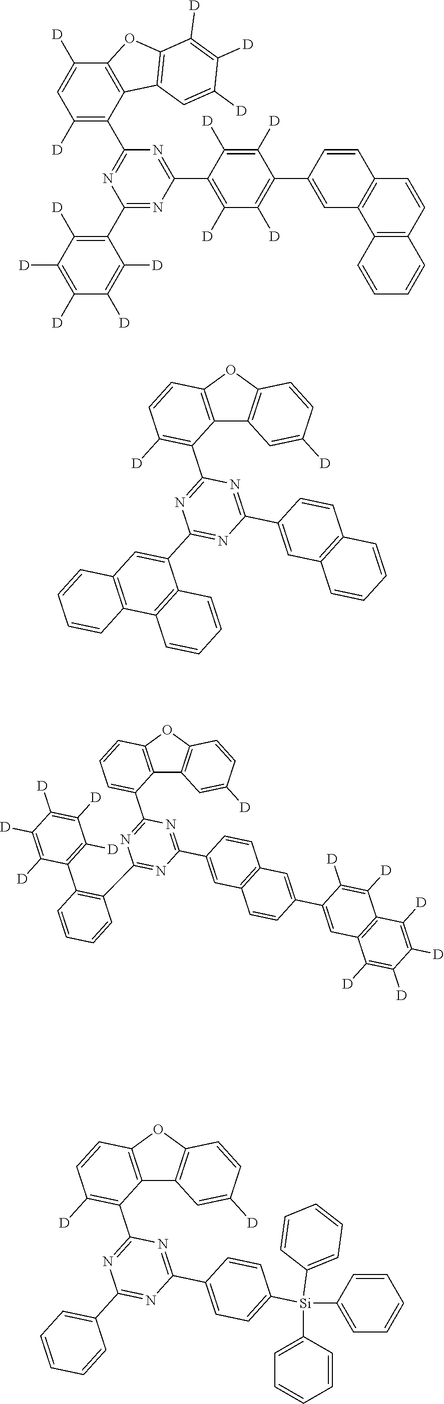 [SMILES: [2H]c1cc2c(oc3c([2H])cc([2H])c(-c4nc(-c5c([2H])c([2H])c([2H])c([2H])c5[2H])nc(-c5c([2H])c([2H])c(-c6ccc7ccc8ccccc8c7c6)c([2H])c5[2H])n4)c32)c([2H])c1[2H] ; [2H]c1ccc2oc3ccc([2H])c(-c4nc(-c5ccc6ccccc6c5)nc(-c5cc6ccccc6c6ccccc56)n4)c3c2c1 ; [2H]c1ccc2oc3cccc(-c4nc(-c5ccc6cc(-c7cc8c([2H])c([2H])c([2H])c([2H])c8c([2H])c7[2H])ccc6c5)nc(-c5ccccc5-c5c([2H])c([2H])c([2H])c([2H])c5[2H])n4)c3c2c1 ; [2H]c1ccc2oc3ccc([2H])c(-c4nc(-c5ccccc5)nc(-c5ccc([Si](c6ccccc6)(c6ccccc6)c6ccccc6)cc5)n4)c3c2c1]

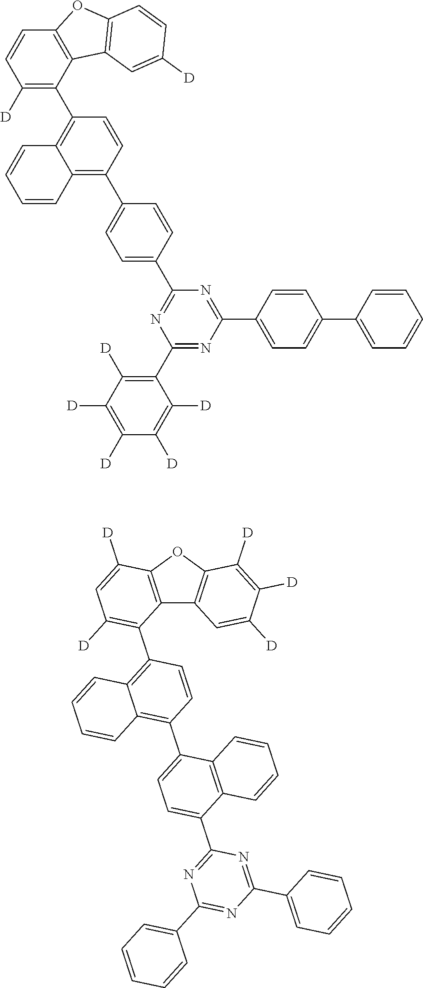 [SMILES: [2H]c1ccc2oc3ccc([2H])c(-c4ccc(-c5ccc(-c6nc(-c7ccc(-c8ccccc8)cc7)nc(-c7c([2H])c([2H])c([2H])c([2H])c7[2H])n6)cc5)c5ccccc45)c3c2c1 ; [2H]c1cc2c(oc3c([2H])cc([2H])c(-c4ccc(-c5ccc(-c6nc(-c7ccccc7)nc(-c7ccccc7)n6)c6ccccc56)c5ccccc45)c32)c([2H])c1[2H]]

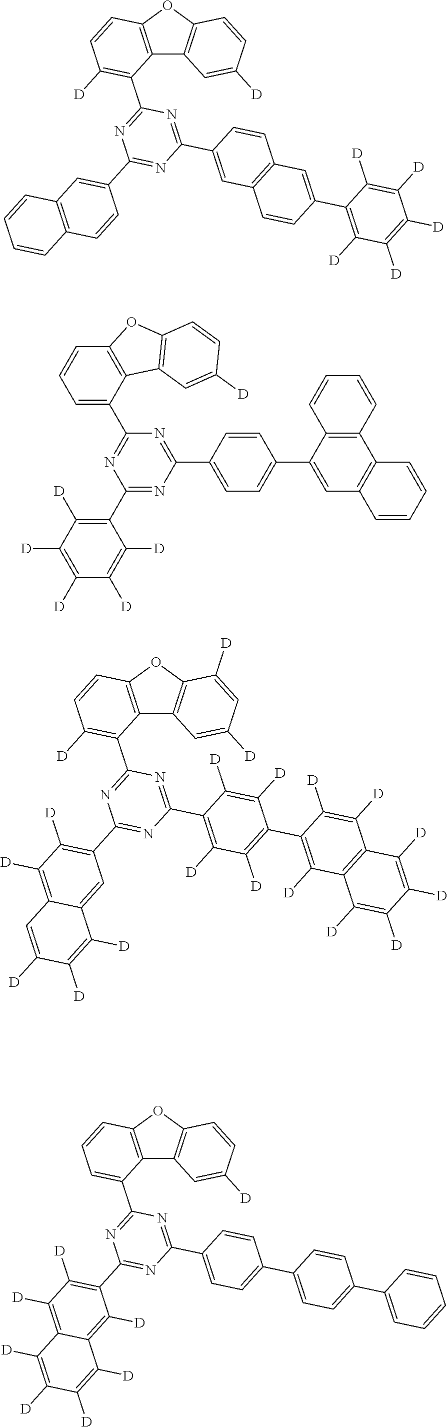

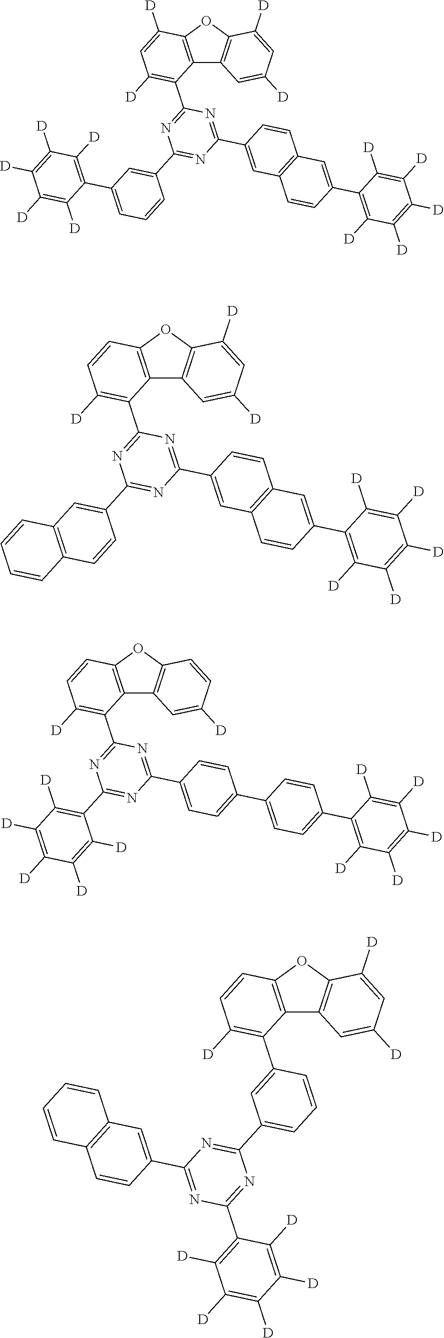

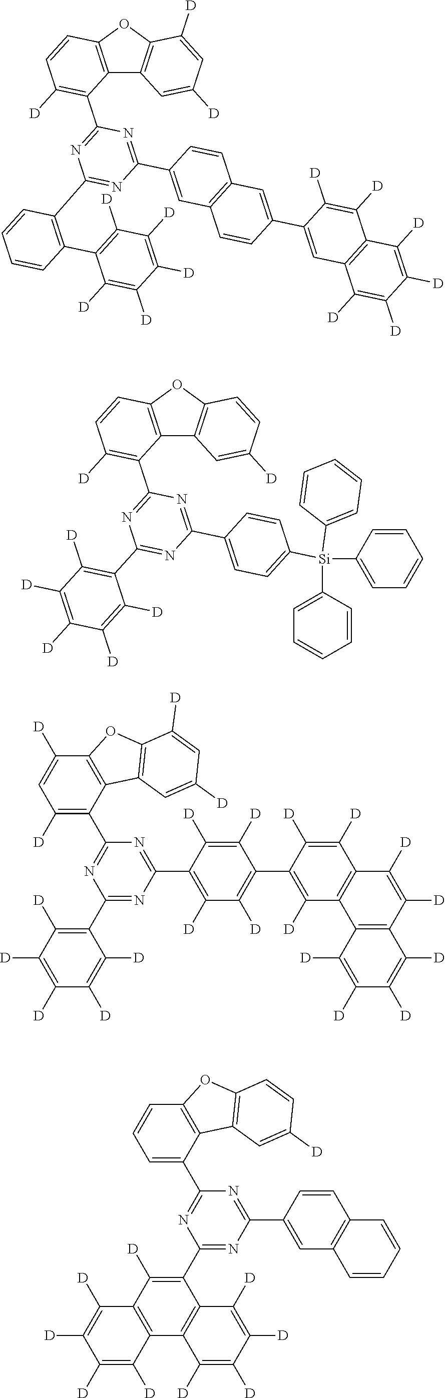

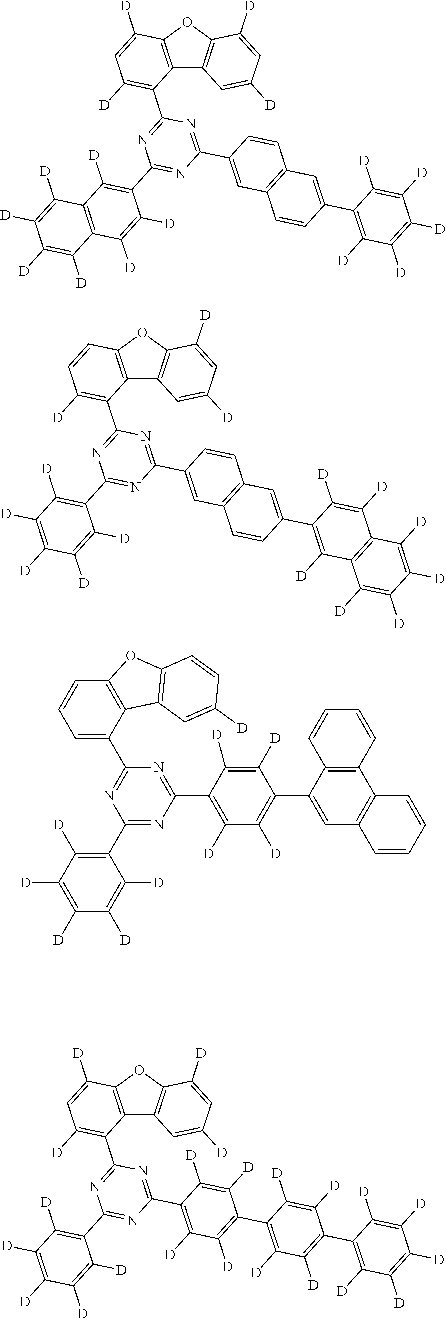

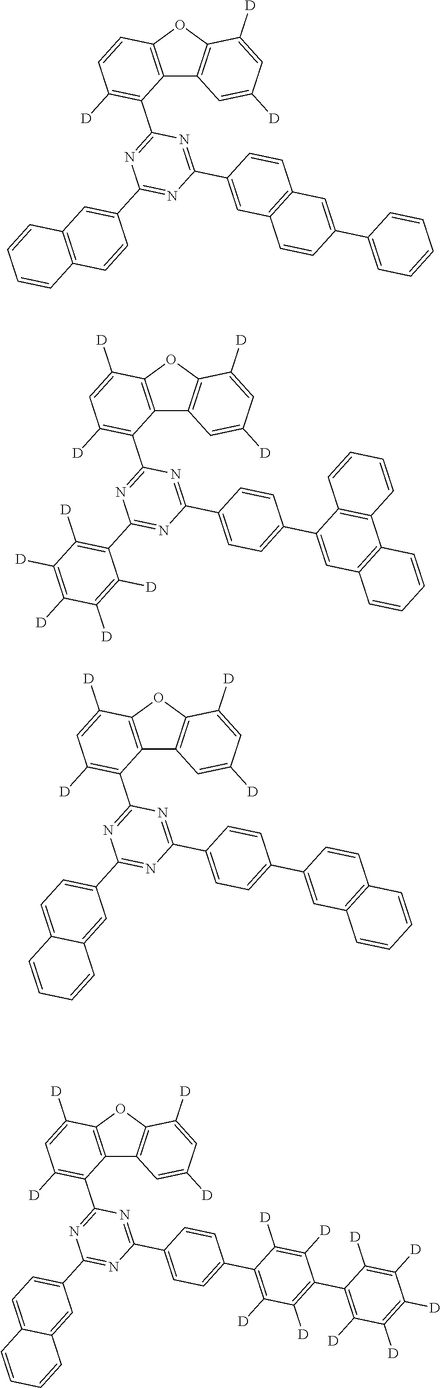

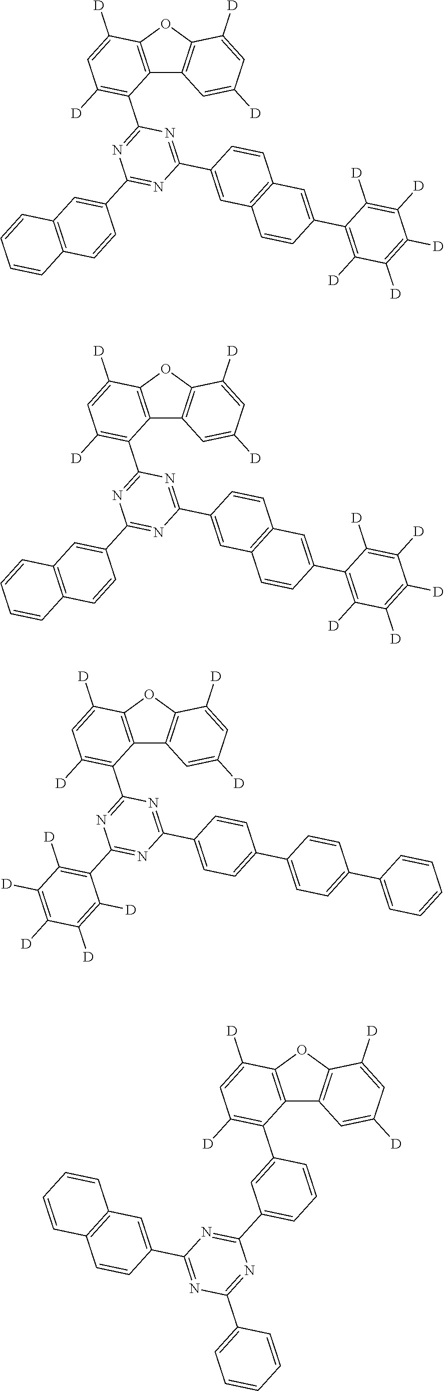 [SMILES: [2H]c1cc([2H])c2oc3c([2H])cc([2H])c(-c4nc(-c5ccc6ccccc6c5)nc(-c5ccc6cc(-c7c([2H])c([2H])c([2H])c([2H])c7[2H])ccc6c5)n4)c3c2c1 ; [2H]c1cc([2H])c2oc3c([2H])cc([2H])c(-c4nc(-c5ccc6ccccc6c5)nc(-c5ccc6cc(-c7c([2H])c([2H])c([2H])c([2H])c7[2H])ccc6c5)n4)c3c2c1 ; [2H]c1cc([2H])c2oc3c([2H])cc([2H])c(-c4nc(-c5ccc(-c6ccc(-c7ccccc7)cc6)cc5)nc(-c5c([2H])c([2H])c([2H])c([2H])c5[2H])n4)c3c2c1 ; [2H]c1cc([2H])c2oc3c([2H])cc([2H])c(-c4cccc(-c5nc(-c6ccccc6)nc(-c6ccc7ccccc7c6)n5)c4)c3c2c1]

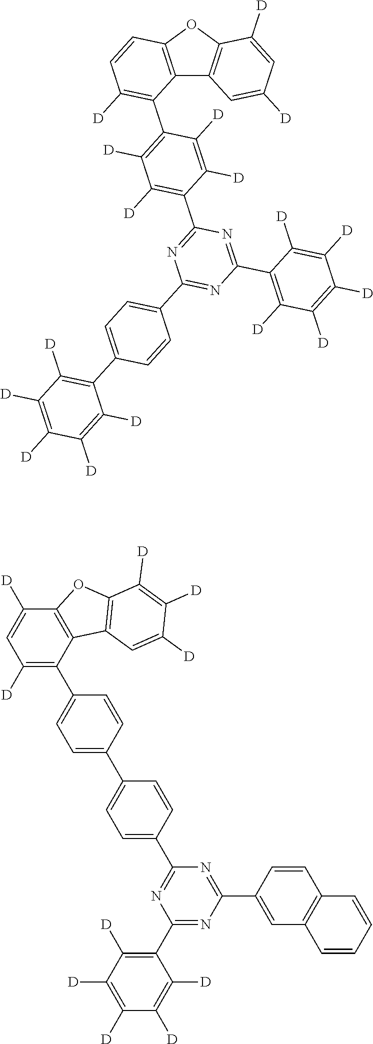 [SMILES: [2H]c1cc([2H])c2oc3ccc([2H])c(-c4c([2H])c([2H])c(-c5nc(-c6ccc(-c7c([2H])c([2H])c([2H])c([2H])c7[2H])cc6)nc(-c6c([2H])c([2H])c([2H])c([2H])c6[2H])n5)c([2H])c4[2H])c3c2c1 ; [2H]c1cc2c(oc3c([2H])cc([2H])c(-c4ccc(-c5ccc(-c6nc(-c7ccc8ccccc8c7)nc(-c7c([2H])c([2H])c([2H])c([2H])c7[2H])n6)cc5)cc4)c32)c([2H])c1[2H]]

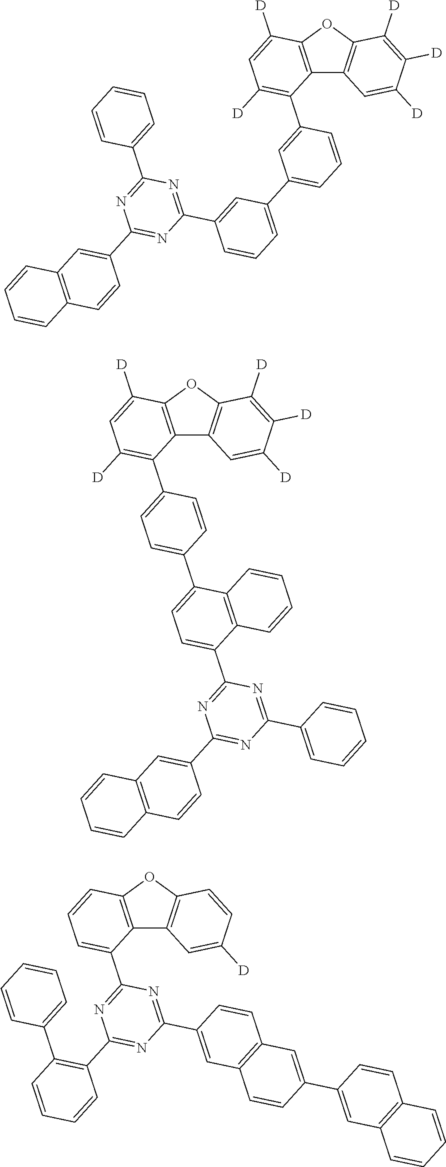 [SMILES: [2H]c1cc2c(oc3c([2H])cc([2H])c(-c4cccc(-c5cccc(-c6nc(-c7ccccc7)nc(-c7ccc8ccccc8c7)n6)c5)c4)c32)c([2H])c1[2H] ; [2H]c1cc2c(oc3c([2H])cc([2H])c(-c4ccc(-c5ccc(-c6nc(-c7ccccc7)nc(-c7ccc8ccccc8c7)n6)c6ccccc56)cc4)c32)c([2H])c1[2H] ; [2H]c1ccc2oc3cccc(-c4nc(-c5ccc6cc(-c7ccc8ccccc8c7)ccc6c5)nc(-c5ccccc5-c5ccccc5)n4)c3c2c1]

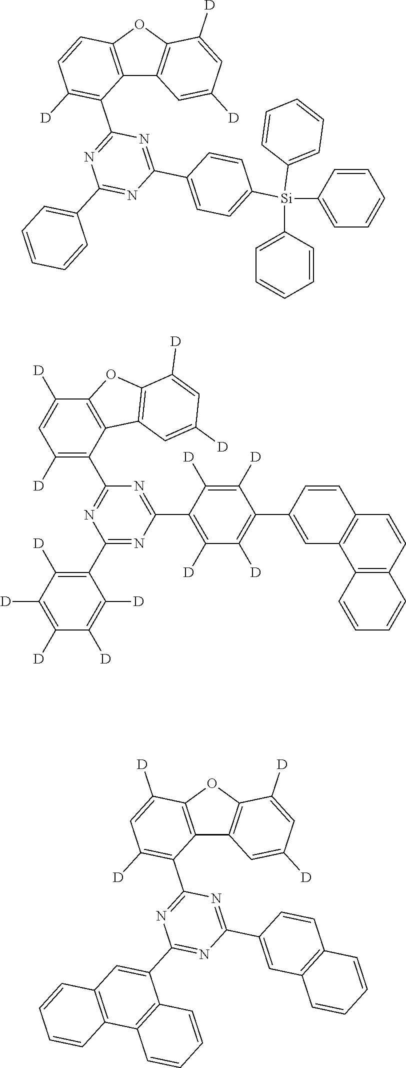 [SMILES: [2H]c1cc([2H])c2oc3ccc([2H])c(-c4nc(-c5ccccc5)nc(-c5ccc([Si](c6ccccc6)(c6ccccc6)c6ccccc6)cc5)n4)c3c2c1 ; [2H]c1cc([2H])c2oc3c([2H])cc([2H])c(-c4nc(-c5c([2H])c([2H])c([2H])c([2H])c5[2H])nc(-c5c([2H])c([2H])c(-c6ccc7ccc8ccccc8c7c6)c([2H])c5[2H])n4)c3c2c1 ; [2H]c1cc([2H])c2oc3c([2H])cc([2H])c(-c4nc(-c5ccc6ccccc6c5)nc(-c5cc6ccccc6c6ccccc56)n4)c3c2c1]

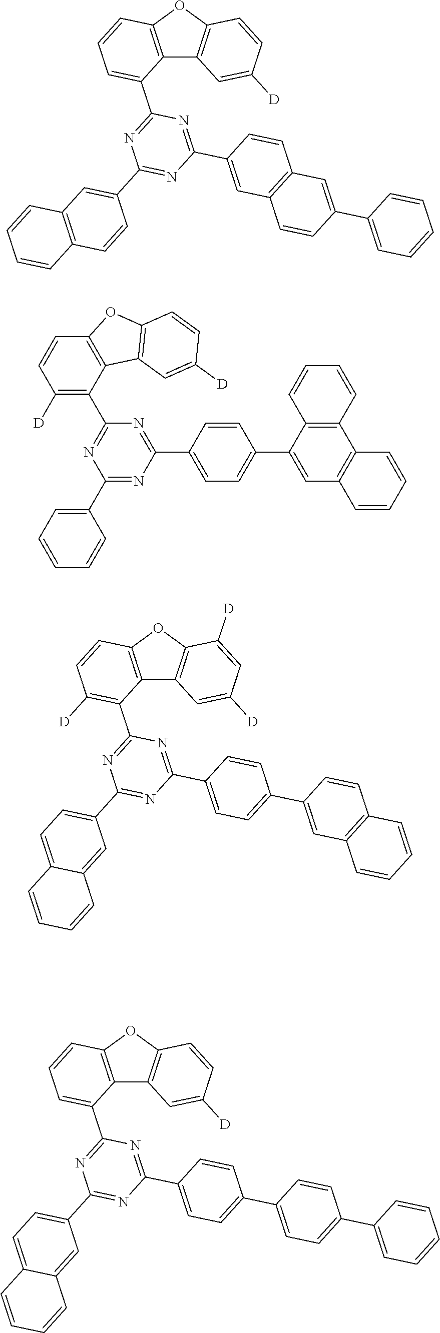 [SMILES: [2H]c1ccc2oc3cccc(-c4nc(-c5ccc6ccccc6c5)nc(-c5ccc6cc(-c7ccccc7)ccc6c5)n4)c3c2c1 ; [2H]c1ccc2oc3ccc([2H])c(-c4nc(-c5ccccc5)nc(-c5ccc(-c6cc7ccccc7c7ccccc67)cc5)n4)c3c2c1 ; [2H]c1cc([2H])c2oc3ccc([2H])c(-c4nc(-c5ccc(-c6ccc7ccccc7c6)cc5)nc(-c5ccc6ccccc6c5)n4)c3c2c1 ; [2H]c1ccc2oc3cccc(-c4nc(-c5ccc(-c6ccc(-c7ccccc7)cc6)cc5)nc(-c5ccc6ccccc6c5)n4)c3c2c1]

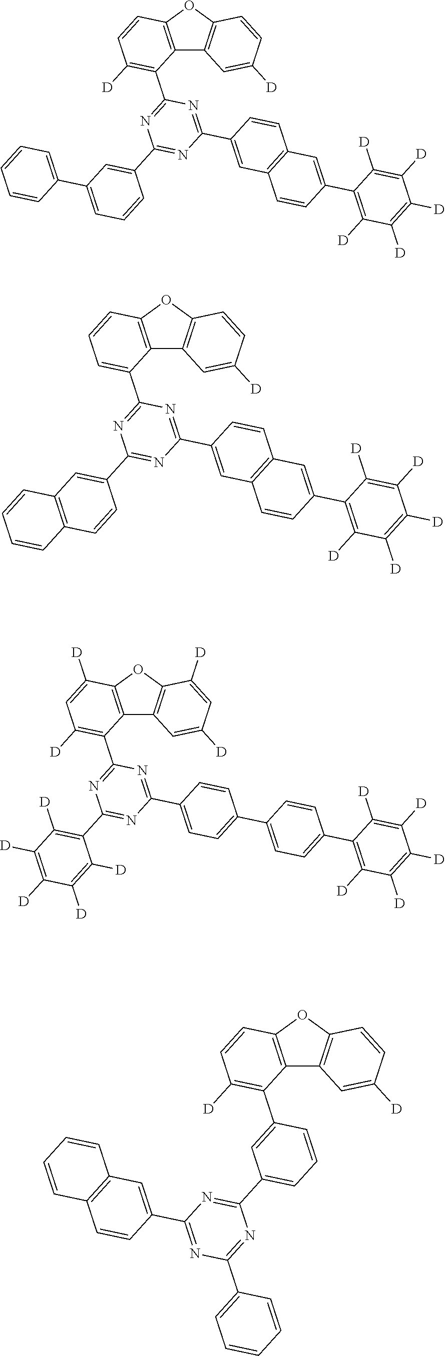 [SMILES: [2H]c1ccc2oc3ccc([2H])c(-c4nc(-c5cccc(-c6ccccc6)c5)nc(-c5ccc6cc(-c7c([2H])c([2H])c([2H])c([2H])c7[2H])ccc6c5)n4)c3c2c1 ; [2H]c1ccc2oc3cccc(-c4nc(-c5ccc6ccccc6c5)nc(-c5ccc6cc(-c7c([2H])c([2H])c([2H])c([2H])c7[2H])ccc6c5)n4)c3c2c1 ; [2H]c1cc([2H])c2oc3c([2H])cc([2H])c(-c4nc(-c5ccc(-c6ccc(-c7c([2H])c([2H])c([2H])c([2H])c7[2H])cc6)cc5)nc(-c5c([2H])c([2H])c([2H])c([2H])c5[2H])n4)c3c2c1 ; [2H]c1ccc2oc3ccc([2H])c(-c4cccc(-c5nc(-c6ccccc6)nc(-c6ccc7ccccc7c6)n5)c4)c3c2c1]

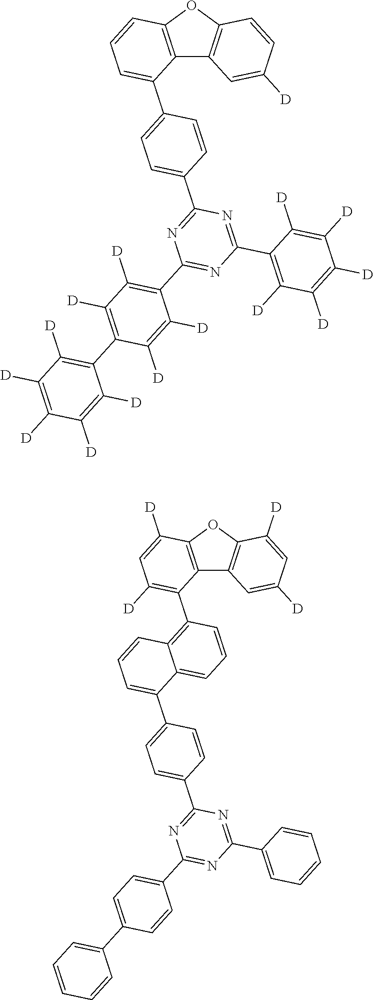 [SMILES: [2H]c1ccc2oc3cccc(-c4ccc(-c5nc(-c6c([2H])c([2H])c([2H])c([2H])c6[2H])nc(-c6c([2H])c([2H])c(-c7c([2H])c([2H])c([2H])c([2H])c7[2H])c([2H])c6[2H])n5)cc4)c3c2c1 ; [2H]c1cc([2H])c2oc3c([2H])cc([2H])c(-c4cccc5c(-c6ccc(-c7nc(-c8ccccc8)nc(-c8ccc(-c9ccccc9)cc8)n7)cc6)cccc45)c3c2c1]

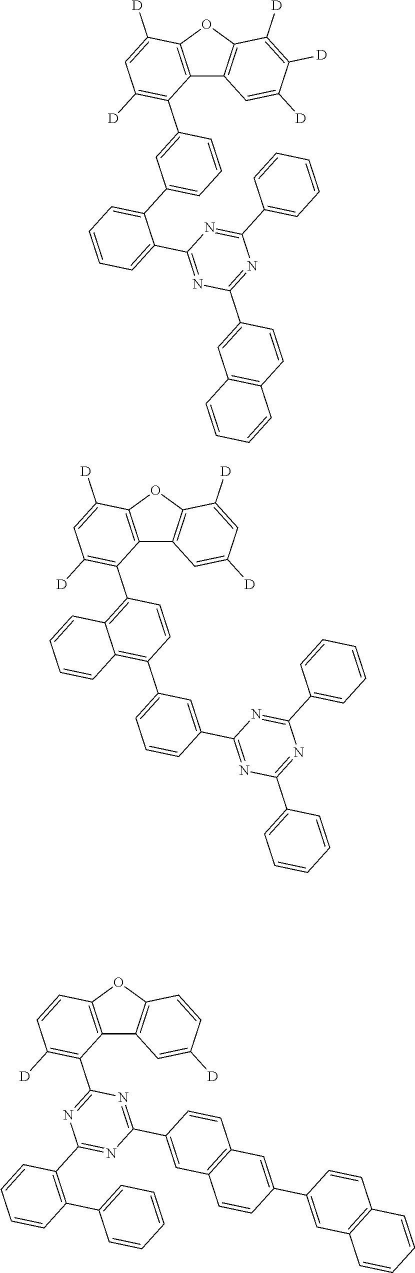

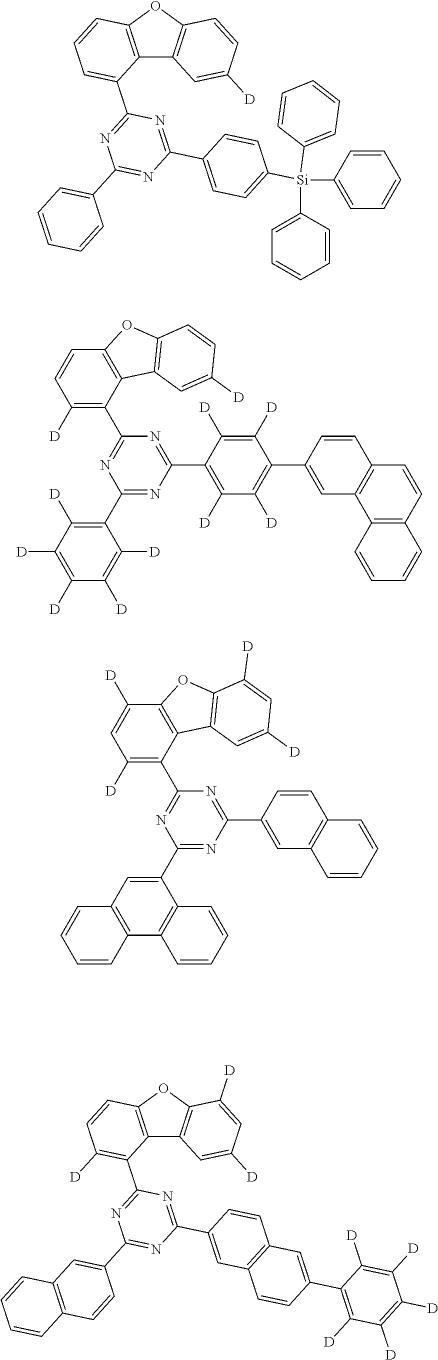 [SMILES: [2H]c1ccc2oc3cccc(-c4nc(-c5ccccc5)nc(-c5ccc([Si](c6ccccc6)(c6ccccc6)c6ccccc6)cc5)n4)c3c2c1 ; [2H]c1ccc2oc3ccc([2H])c(-c4nc(-c5c([2H])c([2H])c([2H])c([2H])c5[2H])nc(-c5c([2H])c([2H])c(-c6ccc7ccc8ccccc8c7c6)c([2H])c5[2H])n4)c3c2c1 ; [2H]c1cc([2H])c2oc3c([2H])cc([2H])c(-c4nc(-c5ccc6ccccc6c5)nc(-c5cc6ccccc6c6ccccc56)n4)c3c2c1 ; [2H]c1cc([2H])c2oc3ccc([2H])c(-c4nc(-c5ccc6ccccc6c5)nc(-c5ccc6cc(-c7c([2H])c([2H])c([2H])c([2H])c7[2H])ccc6c5)n4)c3c2c1]

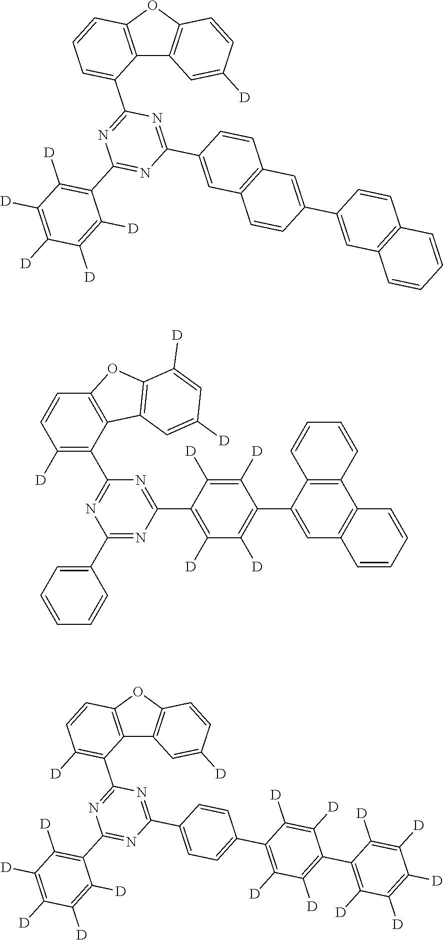 [SMILES: [2H]c1ccc2oc3cccc(-c4nc(-c5ccc6cc(-c7ccc8ccccc8c7)ccc6c5)nc(-c5c([2H])c([2H])c([2H])c([2H])c5[2H])n4)c3c2c1 ; [2H]c1cc([2H])c2oc3ccc([2H])c(-c4nc(-c5ccccc5)nc(-c5c([2H])c([2H])c(-c6cc7ccccc7c7ccccc67)c([2H])c5[2H])n4)c3c2c1 ; [2H]c1ccc2oc3ccc([2H])c(-c4nc(-c5ccc(-c6c([2H])c([2H])c(-c7c([2H])c([2H])c([2H])c([2H])c7[2H])c([2H])c6[2H])cc5)nc(-c5c([2H])c([2H])c([2H])c([2H])c5[2H])n4)c3c2c1]

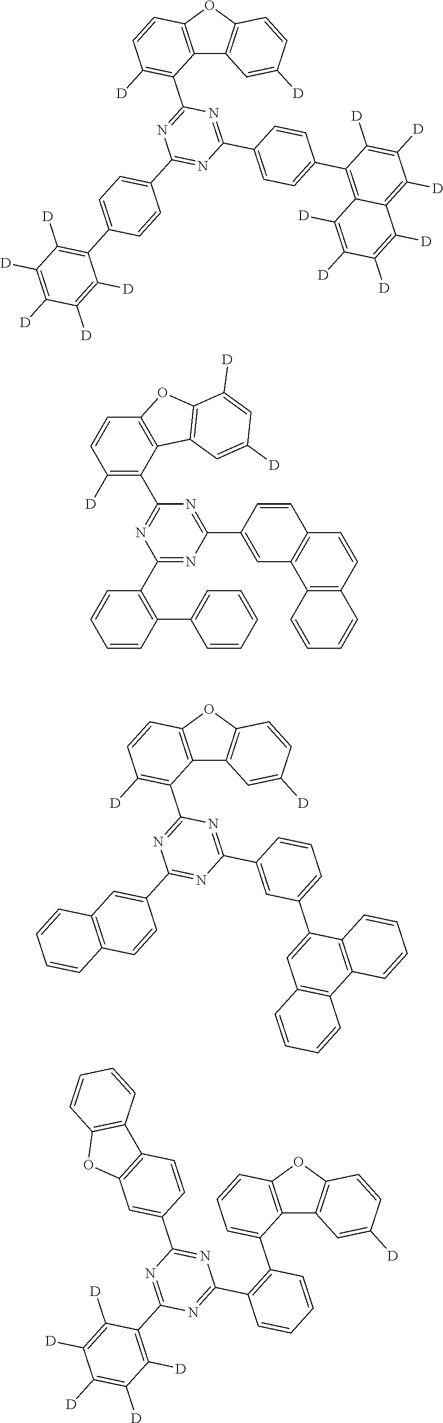 [SMILES: [2H]c1ccc2oc3ccc([2H])c(-c4nc(-c5ccc(-c6c([2H])c([2H])c([2H])c([2H])c6[2H])cc5)nc(-c5ccc(-c6c([2H])c([2H])c([2H])c7c([2H])c([2H])c([2H])c([2H])c67)cc5)n4)c3c2c1 ; [2H]c1cc([2H])c2oc3ccc([2H])c(-c4nc(-c5ccc6ccc7ccccc7c6c5)nc(-c5ccccc5-c5ccccc5)n4)c3c2c1 ; [2H]c1ccc2oc3ccc([2H])c(-c4nc(-c5cccc(-c6cc7ccccc7c7ccccc67)c5)nc(-c5ccc6ccccc6c5)n4)c3c2c1 ; [2H]c1ccc2oc3cccc(-c4ccccc4-c4nc(-c5ccc6c(c5)oc5ccccc56)nc(-c5c([2H])c([2H])c([2H])c([2H])c5[2H])n4)c3c2c1]

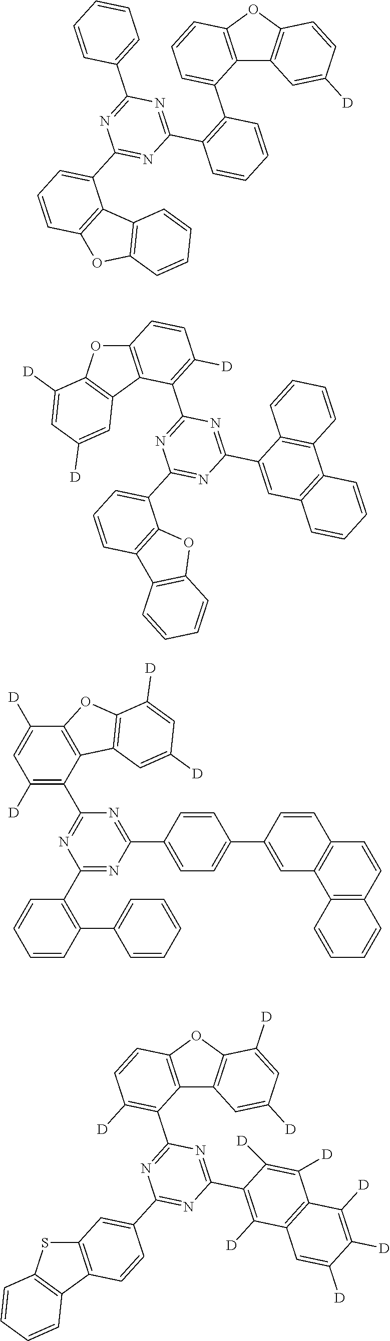

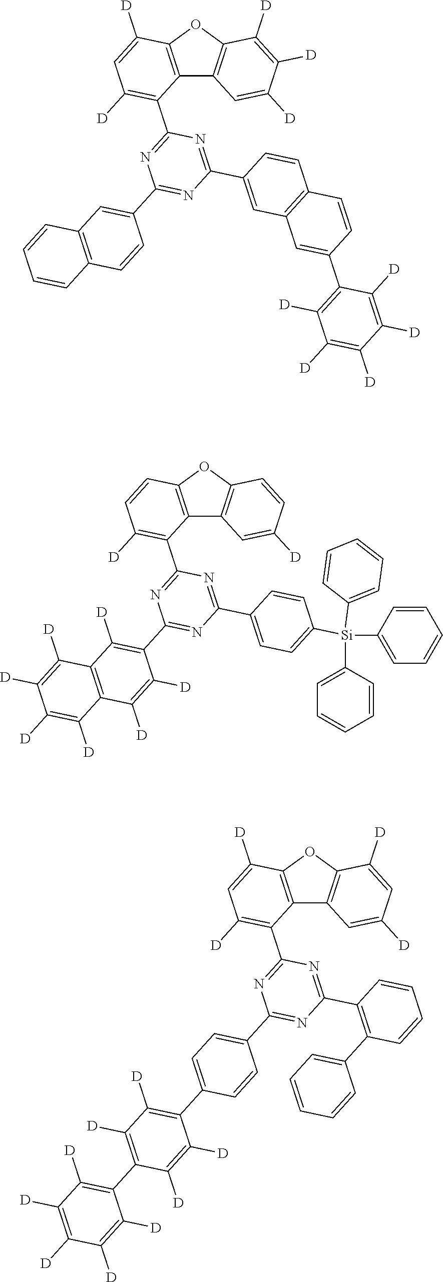 [SMILES: [2H]c1cc2c(oc3c([2H])cc([2H])c(-c4nc(-c5ccc6ccccc6c5)nc(-c5ccc6ccc(-c7c([2H])c([2H])c([2H])c([2H])c7[2H])cc6c5)n4)c32)c([2H])c1[2H] ; [2H]c1ccc2oc3ccc([2H])c(-c4nc(-c5ccc([Si](c6ccccc6)(c6ccccc6)c6ccccc6)cc5)nc(-c5c([2H])c([2H])c6c([2H])c([2H])c([2H])c([2H])c6c5[2H])n4)c3c2c1 ; [2H]c1cc([2H])c2oc3c([2H])cc([2H])c(-c4nc(-c5ccc(-c6c([2H])c([2H])c(-c7c([2H])c([2H])c([2H])c([2H])c7[2H])c([2H])c6[2H])cc5)nc(-c5ccccc5-c5ccccc5)n4)c3c2c1]

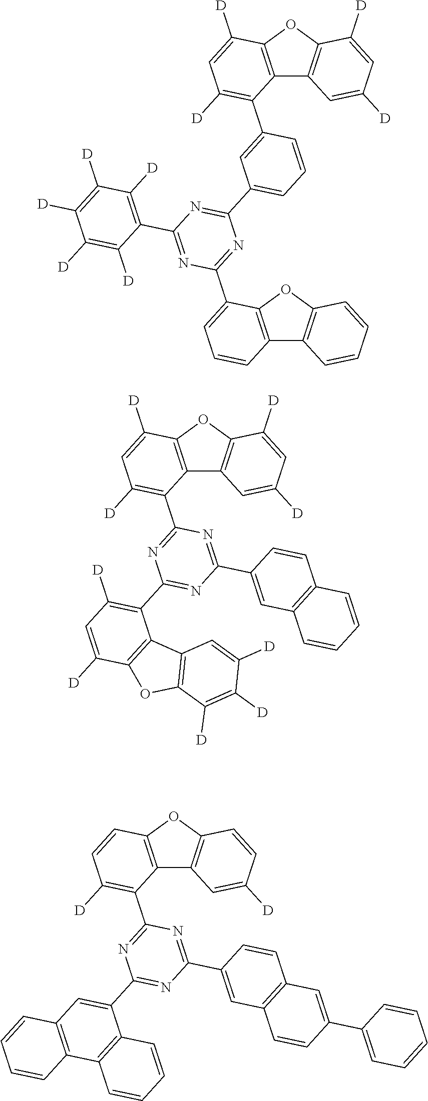

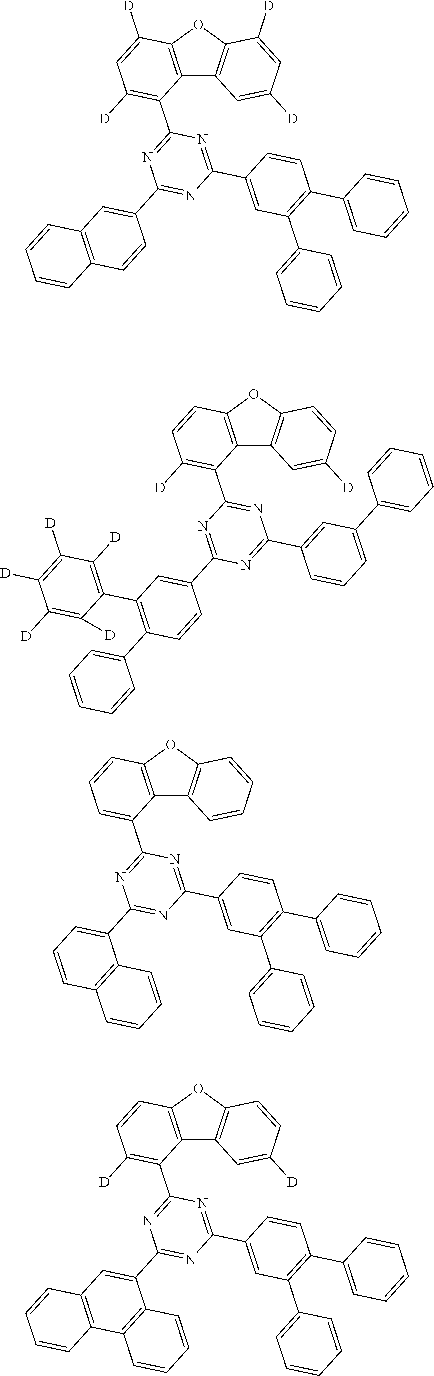

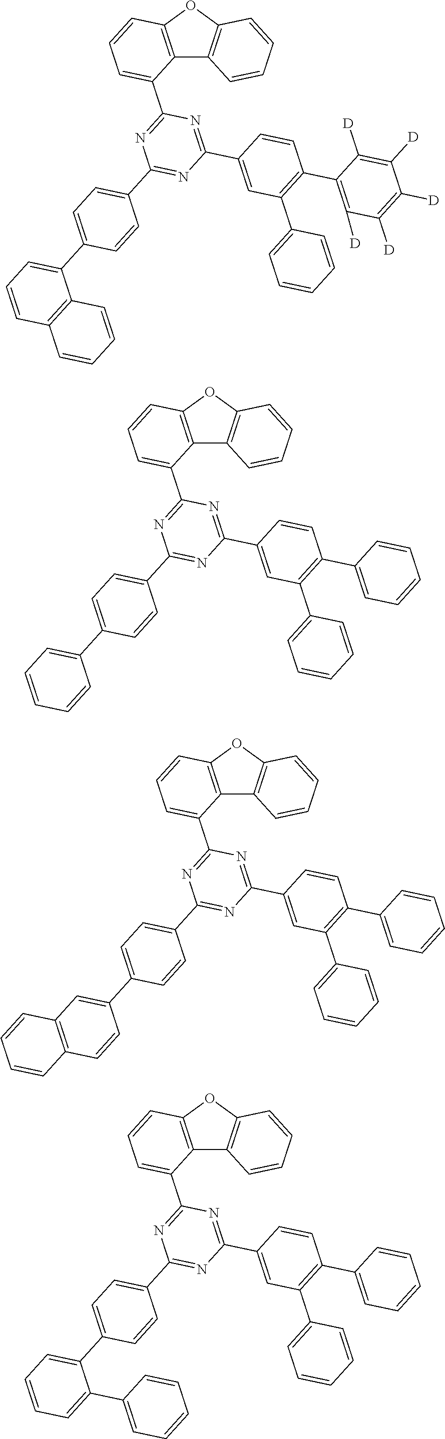 [SMILES: [2H]c1c([2H])c([2H])c(-c2ccc(-c3nc(-c4ccc(-c5cccc6ccccc56)cc4)nc(-c4cccc5oc6ccccc6c45)n3)cc2-c2ccccc2)c([2H])c1[2H] ; c1ccc(-c2ccc(-c3nc(-c4ccc(-c5ccccc5)c(-c5ccccc5)c4)nc(-c4cccc5oc6ccccc6c45)n3)cc2)cc1 ; c1ccc(-c2ccc(-c3nc(-c4ccc(-c5ccc6ccccc6c5)cc4)nc(-c4cccc5oc6ccccc6c45)n3)cc2-c2ccccc2)cc1 ; c1ccc(-c2ccccc2-c2ccc(-c3nc(-c4ccc(-c5ccccc5)c(-c5ccccc5)c4)nc(-c4cccc5oc6ccccc6c45)n3)cc2)cc1]

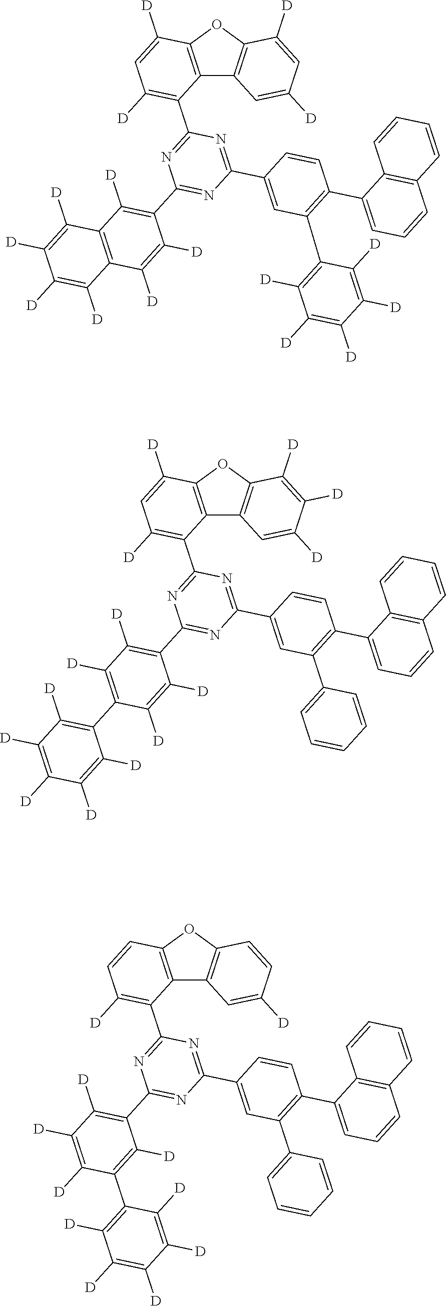

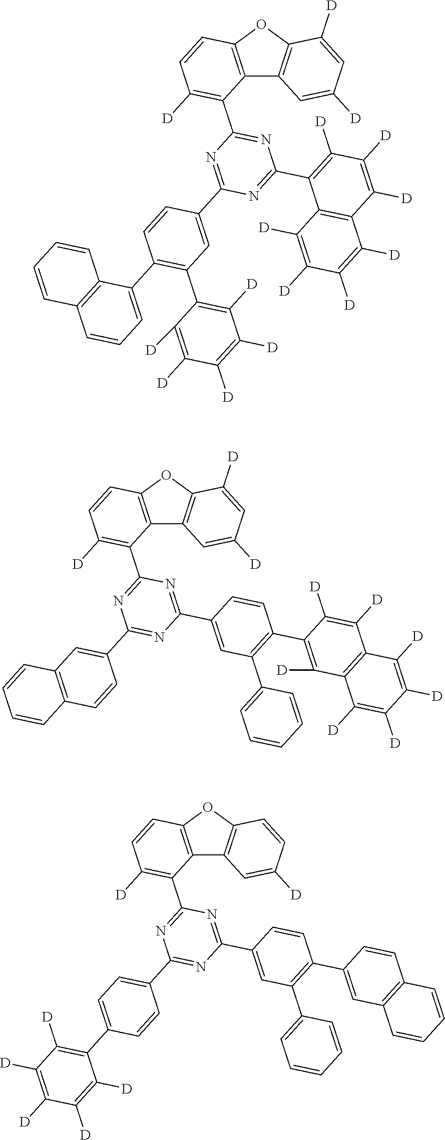 [SMILES: [2H]c1cc([2H])c2oc3ccc([2H])c(-c4nc(-c5ccc(-c6cccc7ccccc67)c(-c6c([2H])c([2H])c([2H])c([2H])c6[2H])c5)nc(-c5c([2H])c([2H])c([2H])c6c([2H])c([2H])c([2H])c([2H])c56)n4)c3c2c1 ; [2H]c1cc([2H])c2oc3ccc([2H])c(-c4nc(-c5ccc(-c6c([2H])c([2H])c7c([2H])c([2H])c([2H])c([2H])c7c6[2H])c(-c6ccccc6)c5)nc(-c5ccc6ccccc6c5)n4)c3c2c1 ; [2H]c1ccc2oc3ccc([2H])c(-c4nc(-c5ccc(-c6c([2H])c([2H])c([2H])c([2H])c6[2H])cc5)nc(-c5ccc(-c6ccc7ccccc7c6)c(-c6ccccc6)c5)n4)c3c2c1]

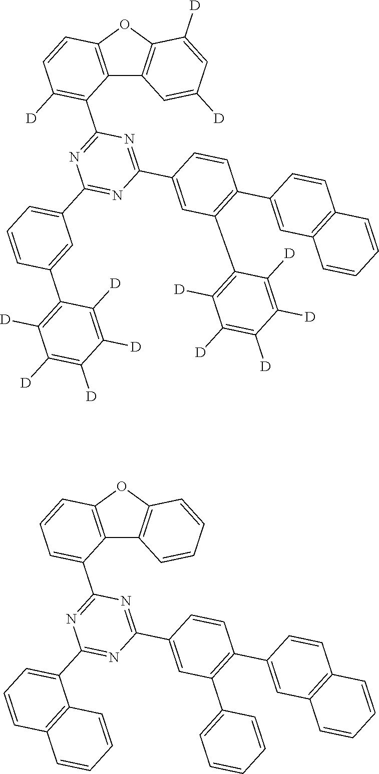 [SMILES: [2H]c1cc([2H])c2oc3ccc([2H])c(-c4nc(-c5cccc(-c6c([2H])c([2H])c([2H])c([2H])c6[2H])c5)nc(-c5ccc(-c6ccc7ccccc7c6)c(-c6c([2H])c([2H])c([2H])c([2H])c6[2H])c5)n4)c3c2c1 ; c1ccc(-c2cc(-c3nc(-c4cccc5ccccc45)nc(-c4cccc5oc6ccccc6c45)n3)ccc2-c2ccc3ccccc3c2)cc1]

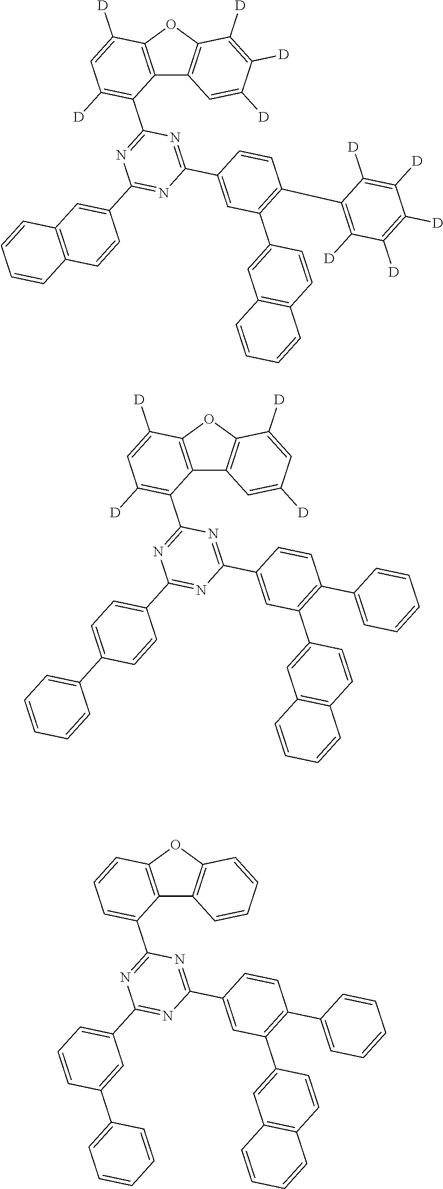

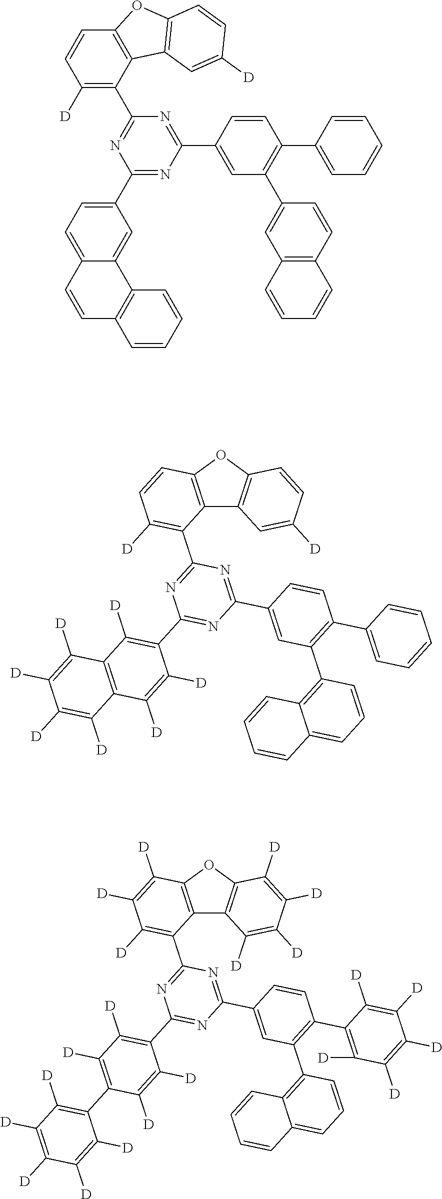 [SMILES: [2H]c1ccc2oc3ccc([2H])c(-c4nc(-c5ccc(-c6ccccc6)c(-c6ccc7ccccc7c6)c5)nc(-c5ccc6ccc7ccccc7c6c5)n4)c3c2c1 ; [2H]c1ccc2oc3ccc([2H])c(-c4nc(-c5ccc(-c6ccccc6)c(-c6cccc7ccccc67)c5)nc(-c5c([2H])c([2H])c6c([2H])c([2H])c([2H])c([2H])c6c5[2H])n4)c3c2c1 ; [2H]c1c([2H])c([2H])c(-c2ccc(-c3nc(-c4c([2H])c([2H])c(-c5c([2H])c([2H])c([2H])c([2H])c5[2H])c([2H])c4[2H])nc(-c4c([2H])c([2H])c([2H])c5oc6c([2H])c([2H])c([2H])c([2H])c6c45)n3)cc2-c2cccc3ccccc23)c([2H])c1[2H]]

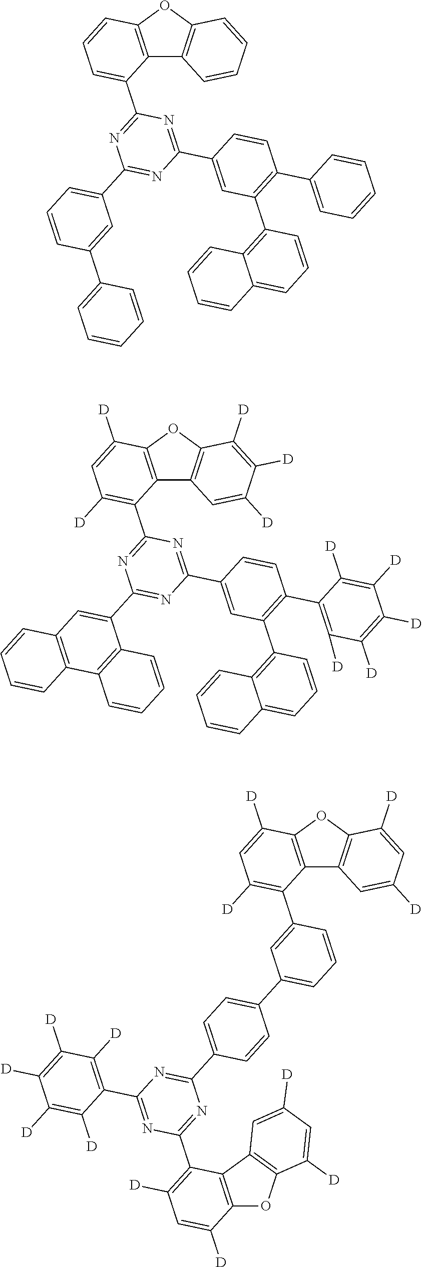 [SMILES: c1ccc(-c2cccc(-c3nc(-c4ccc(-c5ccccc5)c(-c5cccc6ccccc56)c4)nc(-c4cccc5oc6ccccc6c45)n3)c2)cc1 ; [2H]c1cc2c(oc3c([2H])cc([2H])c(-c4nc(-c5ccc(-c6c([2H])c([2H])c([2H])c([2H])c6[2H])c(-c6cccc7ccccc67)c5)nc(-c5cc6ccccc6c6ccccc56)n4)c32)c([2H])c1[2H] ; [2H]c1cc([2H])c2oc3c([2H])cc([2H])c(-c4cccc(-c5ccc(-c6nc(-c7c([2H])c([2H])c([2H])c([2H])c7[2H])nc(-c7c([2H])cc([2H])c8oc9c([2H])cc([2H])cc9c78)n6)cc5)c4)c3c2c1]

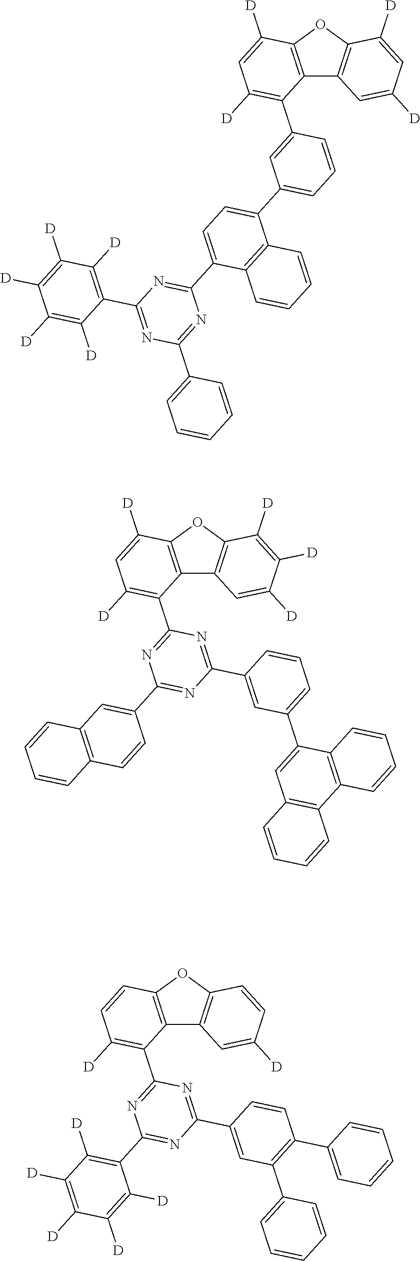 [SMILES: [2H]c1cc([2H])c2oc3c([2H])cc([2H])c(-c4cccc(-c5ccc(-c6nc(-c7ccccc7)nc(-c7c([2H])c([2H])c([2H])c([2H])c7[2H])n6)c6ccccc56)c4)c3c2c1 ; [2H]c1cc2c(oc3c([2H])cc([2H])c(-c4nc(-c5cccc(-c6cc7ccccc7c7ccccc67)c5)nc(-c5ccc6ccccc6c5)n4)c32)c([2H])c1[2H] ; [2H]c1ccc2oc3ccc([2H])c(-c4nc(-c5ccc(-c6ccccc6)c(-c6ccccc6)c5)nc(-c5c([2H])c([2H])c([2H])c([2H])c5[2H])n4)c3c2c1]

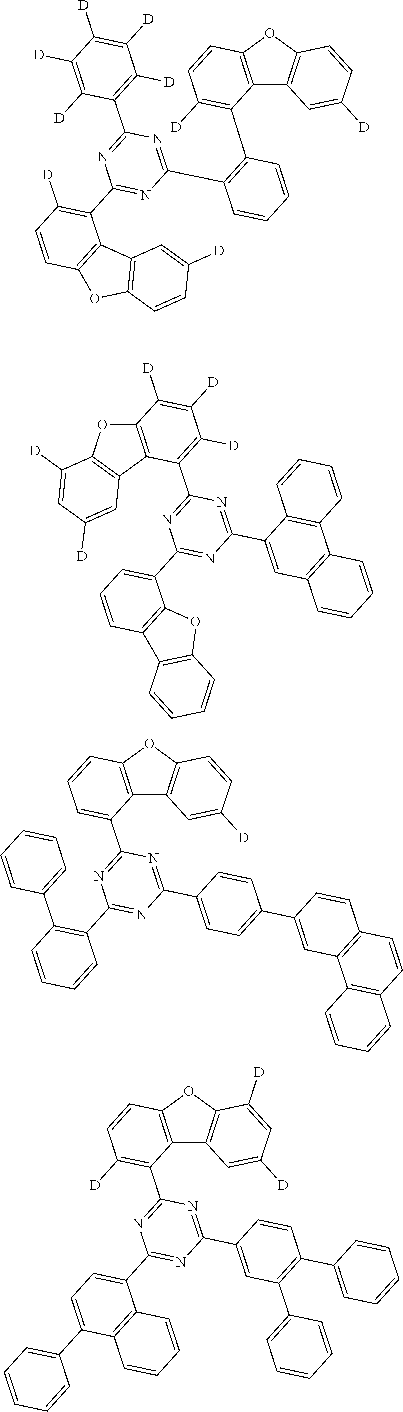

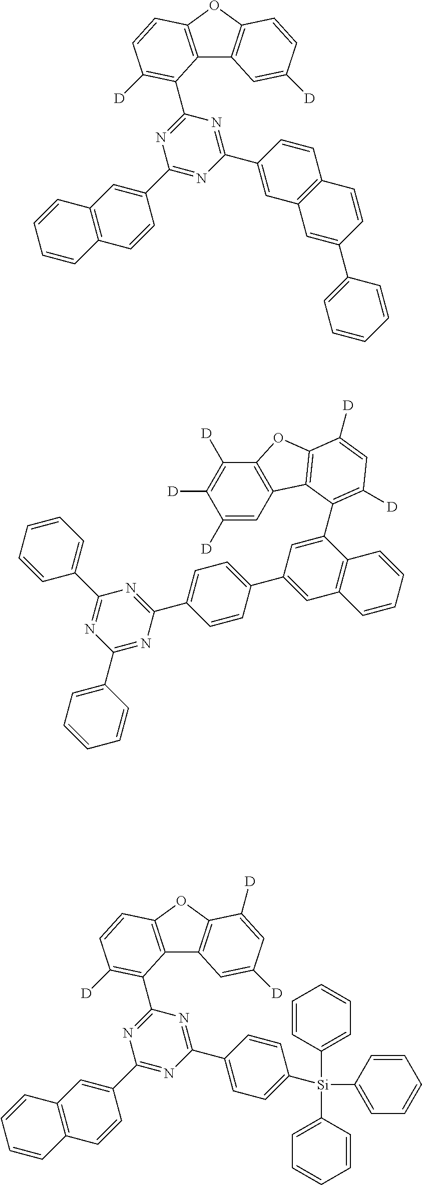 [SMILES: [2H]c1ccc2oc3ccc([2H])c(-c4nc(-c5ccc6ccccc6c5)nc(-c5ccc6ccc(-c7ccccc7)cc6c5)n4)c3c2c1 ; [2H]c1cc2c(oc3c([2H])cc([2H])c(-c4cc(-c5ccc(-c6nc(-c7ccccc7)nc(-c7ccccc7)n6)cc5)cc5ccccc45)c32)c([2H])c1[2H] ; [2H]c1cc([2H])c2oc3ccc([2H])c(-c4nc(-c5ccc([Si](c6ccccc6)(c6ccccc6)c6ccccc6)cc5)nc(-c5ccc6ccccc6c5)n4)c3c2c1]

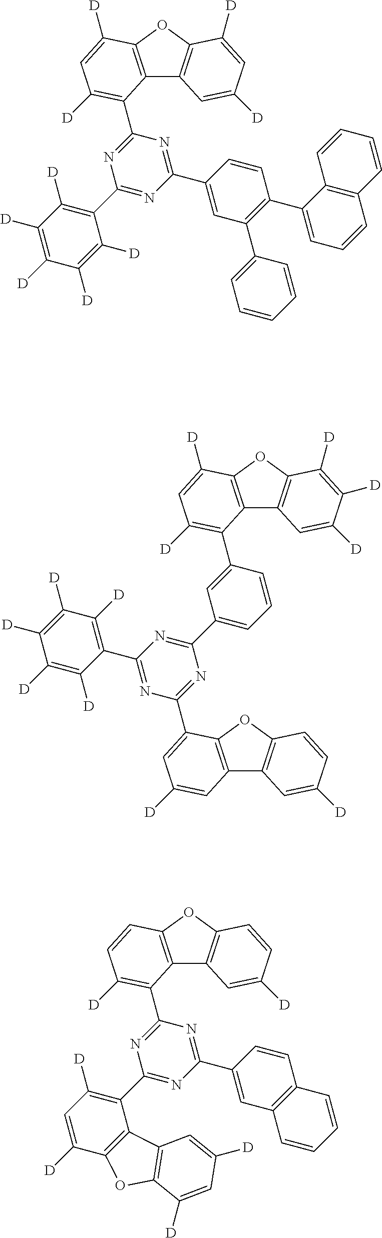 [SMILES: [2H]c1cc([2H])c2oc3c([2H])cc([2H])c(-c4nc(-c5ccc(-c6cccc7ccccc67)c(-c6ccccc6)c5)nc(-c5c([2H])c([2H])c([2H])c([2H])c5[2H])n4)c3c2c1 ; [2H]c1ccc2oc3c(-c4nc(-c5cccc(-c6c([2H])cc([2H])c7oc8c([2H])c([2H])c([2H])cc8c67)c5)nc(-c5c([2H])c([2H])c([2H])c([2H])c5[2H])n4)cc([2H])cc3c2c1 ; [2H]c1ccc2oc3ccc([2H])c(-c4nc(-c5ccc6ccccc6c5)nc(-c5c([2H])cc([2H])c6oc7c([2H])cc([2H])cc7c56)n4)c3c2c1]

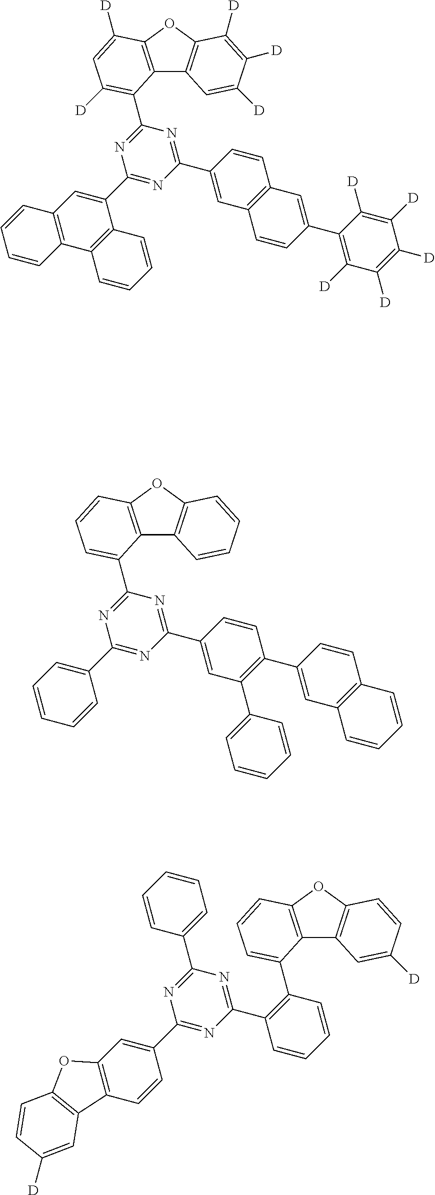 [SMILES: [2H]c1cc2c(oc3c([2H])cc([2H])c(-c4nc(-c5ccc6cc(-c7c([2H])c([2H])c([2H])c([2H])c7[2H])ccc6c5)nc(-c5cc6ccccc6c6ccccc56)n4)c32)c([2H])c1[2H] ; c1ccc(-c2nc(-c3ccc(-c4ccc5ccccc5c4)c(-c4ccccc4)c3)nc(-c3cccc4oc5ccccc5c34)n2)cc1 ; [2H]c1ccc2oc3cc(-c4nc(-c5ccccc5)nc(-c5ccccc5-c5cccc6oc7ccc([2H])cc7c56)n4)ccc3c2c1]

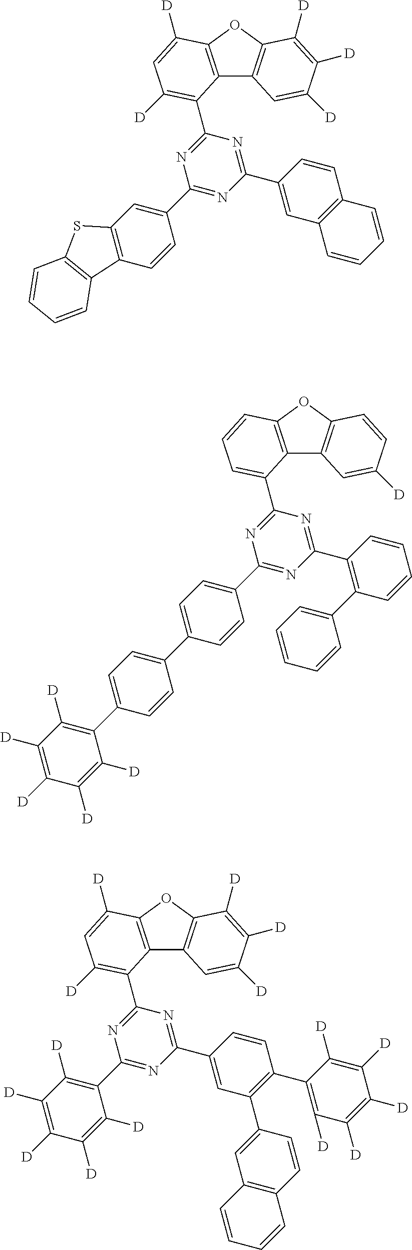 [SMILES: [2H]c1cc2c(oc3c([2H])cc([2H])c(-c4nc(-c5ccc6ccccc6c5)nc(-c5ccc6c(c5)sc5ccccc56)n4)c32)c([2H])c1[2H] ; [2H]c1ccc2oc3cccc(-c4nc(-c5ccc(-c6ccc(-c7c([2H])c([2H])c([2H])c([2H])c7[2H])cc6)cc5)nc(-c5ccccc5-c5ccccc5)n4)c3c2c1 ; [2H]c1cc2c(oc3c([2H])cc([2H])c(-c4nc(-c5ccc(-c6c([2H])c([2H])c([2H])c([2H])c6[2H])c(-c6ccc7ccccc7c6)c5)nc(-c5c([2H])c([2H])c([2H])c([2H])c5[2H])n4)c32)c([2H])c1[2H]]

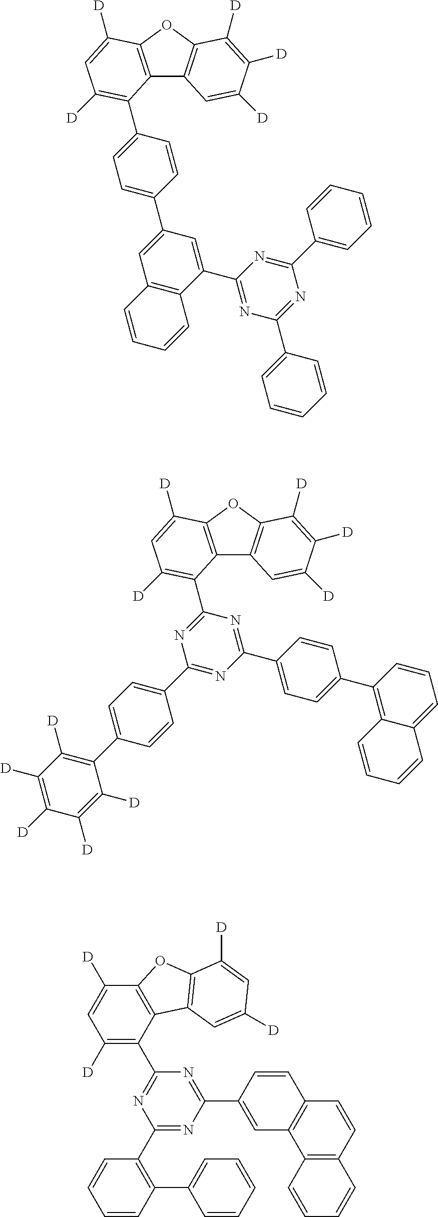 [SMILES: [2H]c1cc2c(oc3c([2H])cc([2H])c(-c4ccc(-c5cc(-c6nc(-c7ccccc7)nc(-c7ccccc7)n6)c6ccccc6c5)cc4)c32)c([2H])c1[2H] ; [2H]c1cc2c(oc3c([2H])cc([2H])c(-c4nc(-c5ccc(-c6c([2H])c([2H])c([2H])c([2H])c6[2H])cc5)nc(-c5ccc(-c6cccc7ccccc67)cc5)n4)c32)c([2H])c1[2H] ; [2H]c1cc([2H])c2oc3c([2H])cc([2H])c(-c4nc(-c5ccc6ccc7ccccc7c6c5)nc(-c5ccccc5-c5ccccc5)n4)c3c2c1]

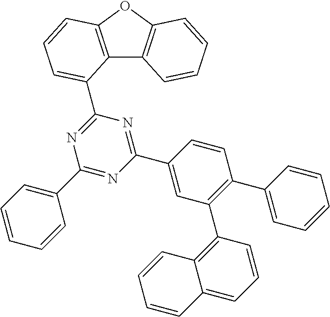 [SMILES: c1ccc(-c2nc(-c3ccc(-c4ccccc4)c(-c4cccc5ccccc45)c3)nc(-c3cccc4oc5ccccc5c34)n2)cc1]

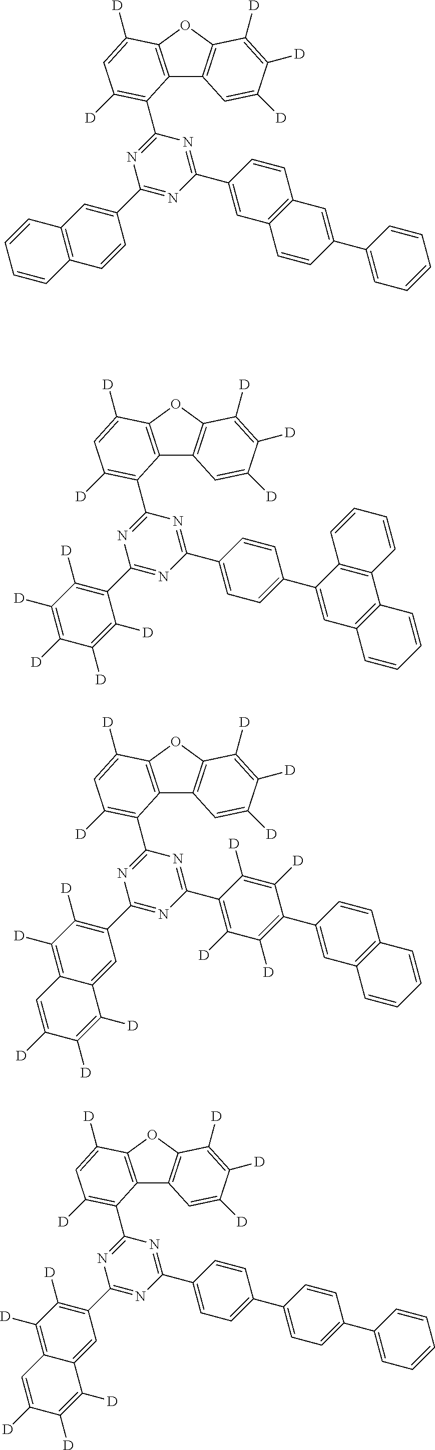

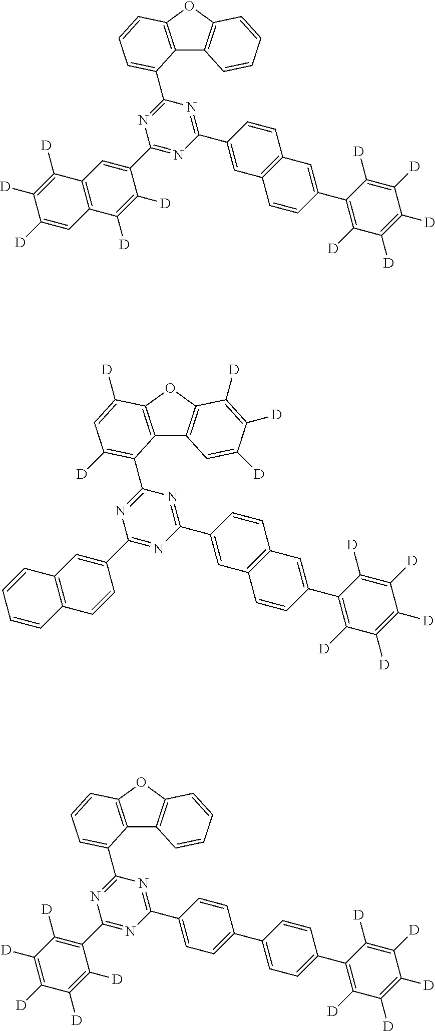 [SMILES: [2H]c1cc2c([2H])c([2H])c(-c3nc(-c4ccc5cc(-c6c([2H])c([2H])c([2H])c([2H])c6[2H])ccc5c4)nc(-c4cccc5oc6ccccc6c45)n3)cc2c([2H])c1[2H] ; [2H]c1cc2c(oc3c([2H])cc([2H])c(-c4nc(-c5ccc6ccccc6c5)nc(-c5ccc6cc(-c7c([2H])c([2H])c([2H])c([2H])c7[2H])ccc6c5)n4)c32)c([2H])c1[2H] ; [2H]c1c([2H])c([2H])c(-c2ccc(-c3ccc(-c4nc(-c5c([2H])c([2H])c([2H])c([2H])c5[2H])nc(-c5cccc6oc7ccccc7c56)n4)cc3)cc2)c([2H])c1[2H]]

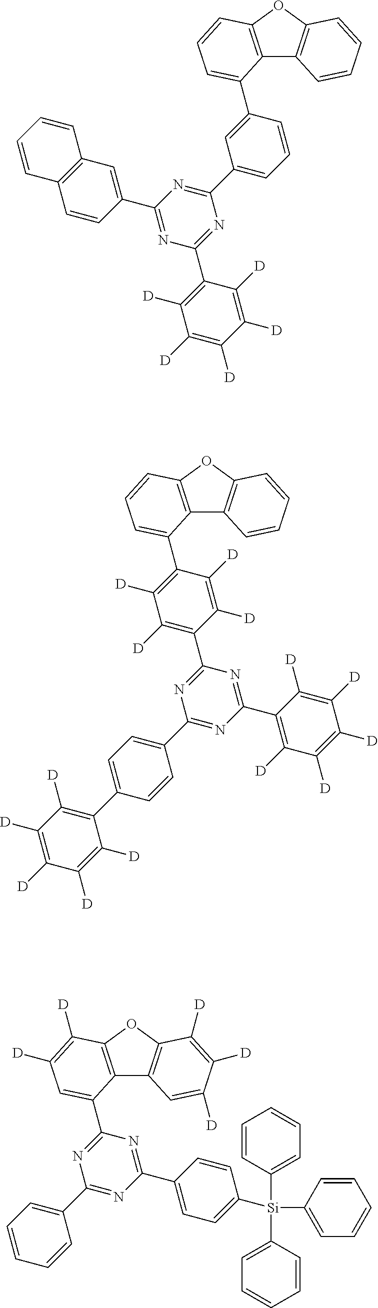 [SMILES: [2H]c1c([2H])c([2H])c(-c2nc(-c3cccc(-c4cccc5oc6ccccc6c45)c3)nc(-c3ccc4ccccc4c3)n2)c([2H])c1[2H] ; [2H]c1c([2H])c([2H])c(-c2ccc(-c3nc(-c4c([2H])c([2H])c([2H])c([2H])c4[2H])nc(-c4c([2H])c([2H])c(-c5cccc6oc7ccccc7c56)c([2H])c4[2H])n3)cc2)c([2H])c1[2H] ; [2H]c1cc2c(oc3c([2H])c([2H])cc(-c4nc(-c5ccccc5)nc(-c5ccc([Si](c6ccccc6)(c6ccccc6)c6ccccc6)cc5)n4)c32)c([2H])c1[2H]]

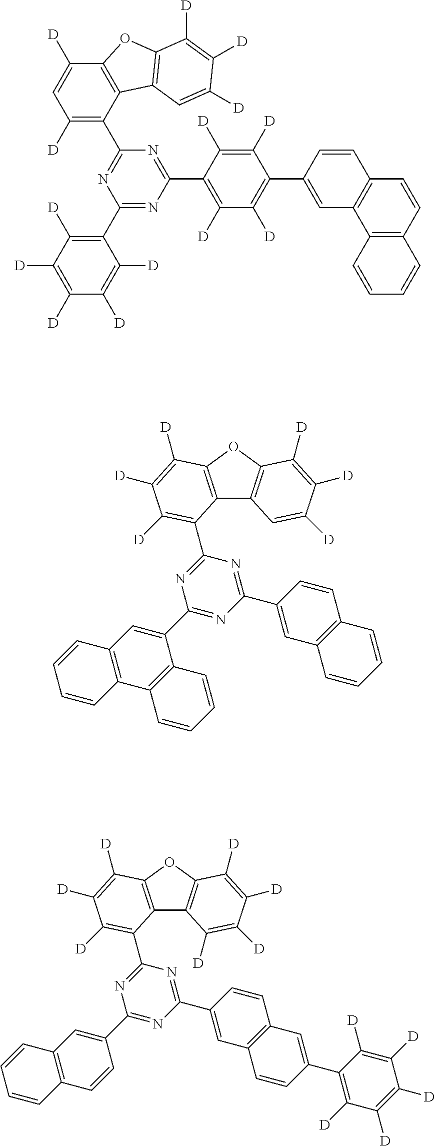 [SMILES: [2H]c1cc2c(oc3c([2H])cc([2H])c(-c4nc(-c5c([2H])c([2H])c([2H])c([2H])c5[2H])nc(-c5c([2H])c([2H])c(-c6ccc7ccc8ccccc8c7c6)c([2H])c5[2H])n4)c32)c([2H])c1[2H] ; [2H]c1cc2c(oc3c([2H])c([2H])c([2H])c(-c4nc(-c5ccc6ccccc6c5)nc(-c5cc6ccccc6c6ccccc56)n4)c32)c([2H])c1[2H] ; [2H]c1c([2H])c([2H])c(-c2ccc3cc(-c4nc(-c5ccc6ccccc6c5)nc(-c5c([2H])c([2H])c([2H])c6oc7c([2H])c([2H])c([2H])c([2H])c7c56)n4)ccc3c2)c([2H])c1[2H]]

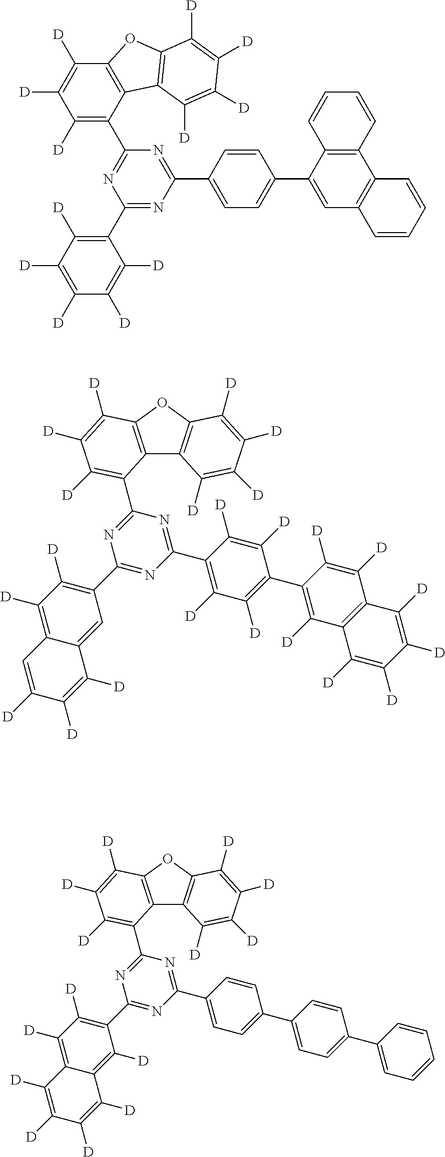

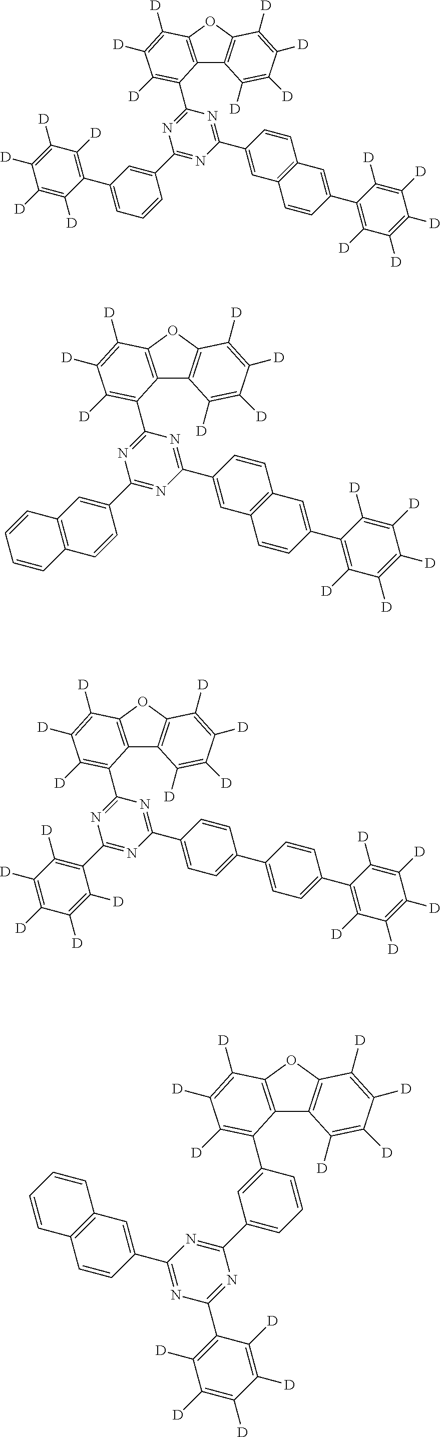

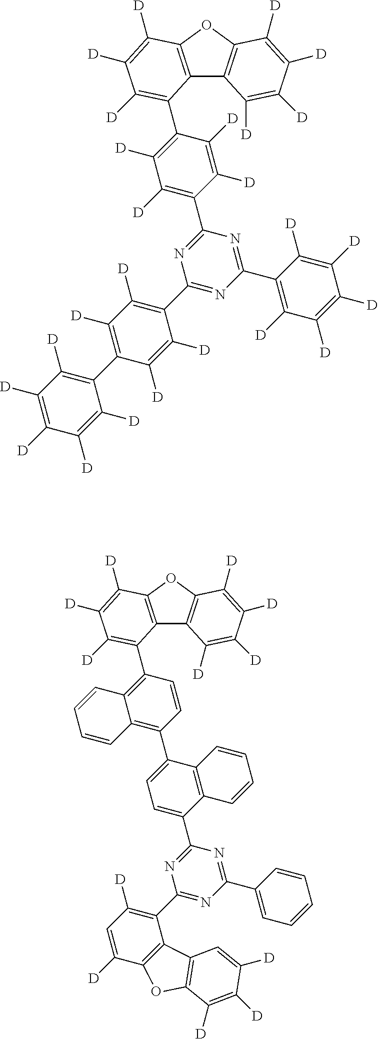 [SMILES: [2H]c1c([2H])c([2H])c(-c2nc(-c3c([2H])c([2H])c(-c4c([2H])c([2H])c([2H])c([2H])c4[2H])c([2H])c3[2H])nc(-c3c([2H])c([2H])c(-c4c([2H])c([2H])c([2H])c5oc6c([2H])c([2H])c([2H])c([2H])c6c45)c([2H])c3[2H])n2)c([2H])c1[2H] ; [2H]c1cc2c(oc3c([2H])cc([2H])c(-c4nc(-c5ccccc5)nc(-c5ccc(-c6ccc(-c7c([2H])c([2H])c([2H])c8oc9c([2H])c([2H])c([2H])c([2H])c9c78)c7ccccc67)c6ccccc56)n4)c32)c([2H])c1[2H]]

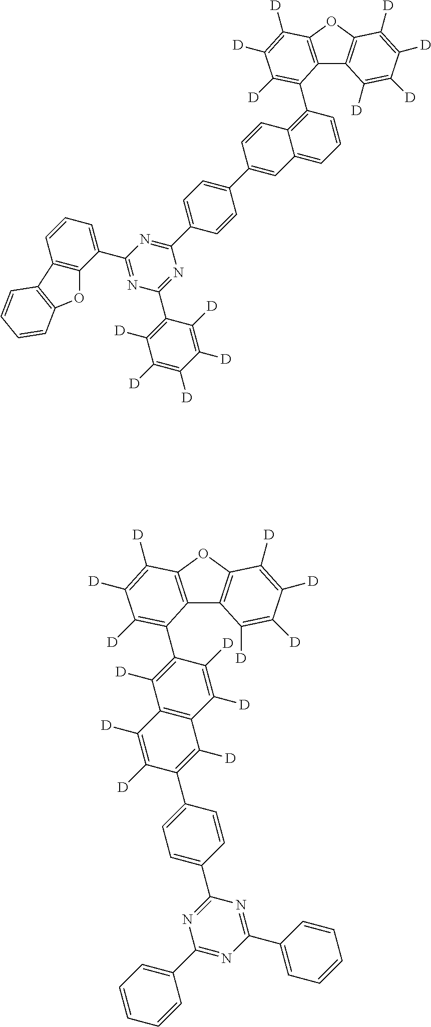 [SMILES: [2H]c1c([2H])c([2H])c(-c2nc(-c3ccc(-c4ccc5c(-c6c([2H])c([2H])c([2H])c7oc8c([2H])c([2H])c([2H])c([2H])c8c67)cccc5c4)cc3)nc(-c3cccc4c3oc3ccccc34)n2)c([2H])c1[2H] ; [2H]c1c([2H])c([2H])c2c(oc3c([2H])c([2H])c([2H])c(-c4c([2H])c([2H])c5c([2H])c(-c6ccc(-c7nc(-c8ccccc8)nc(-c8ccccc8)n7)cc6)c([2H])c([2H])c5c4[2H])c32)c1[2H]]

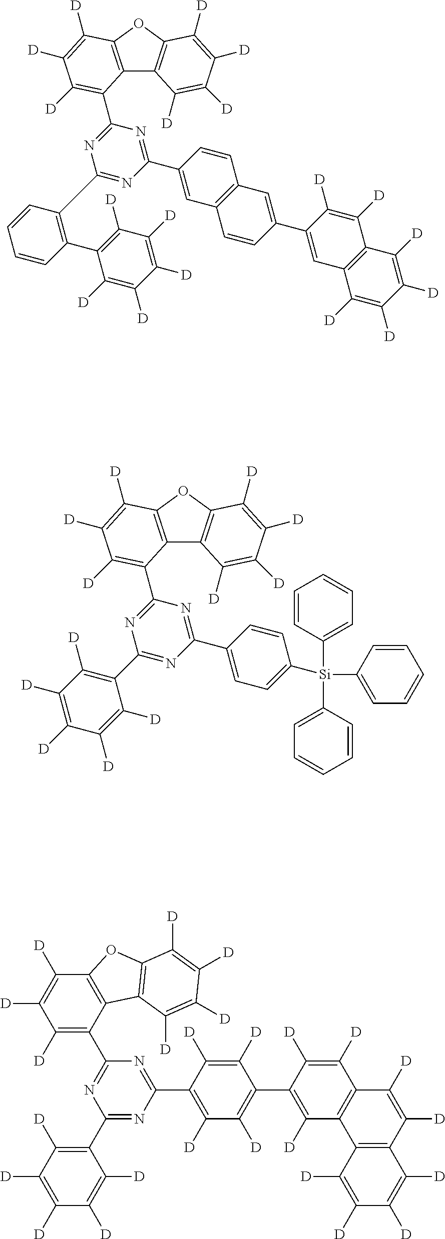

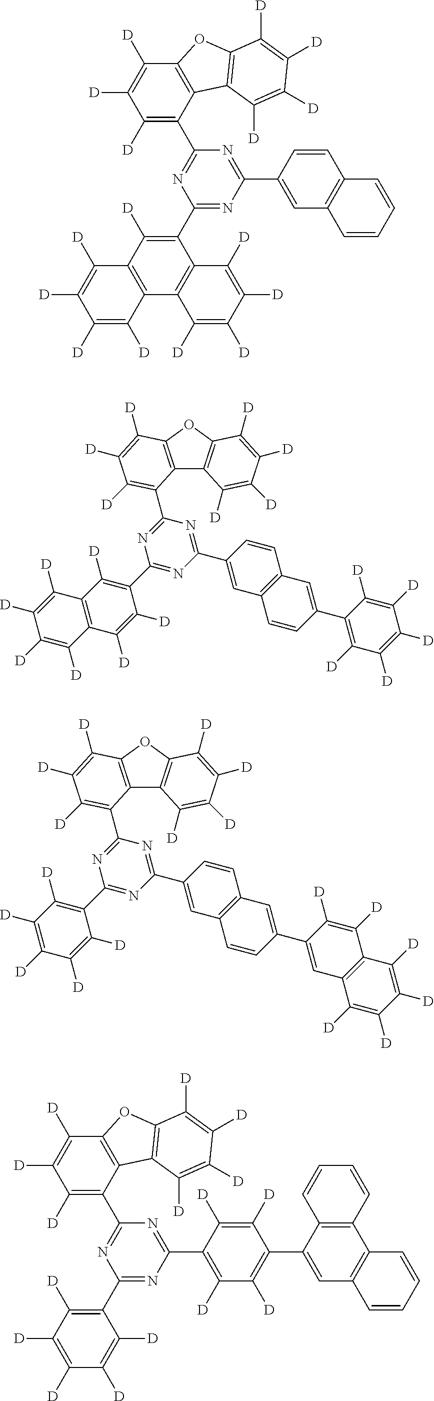

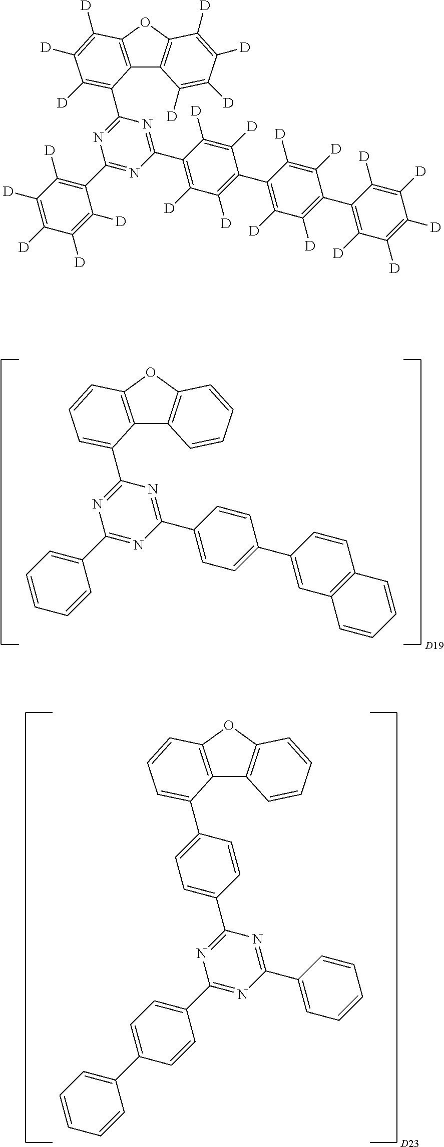

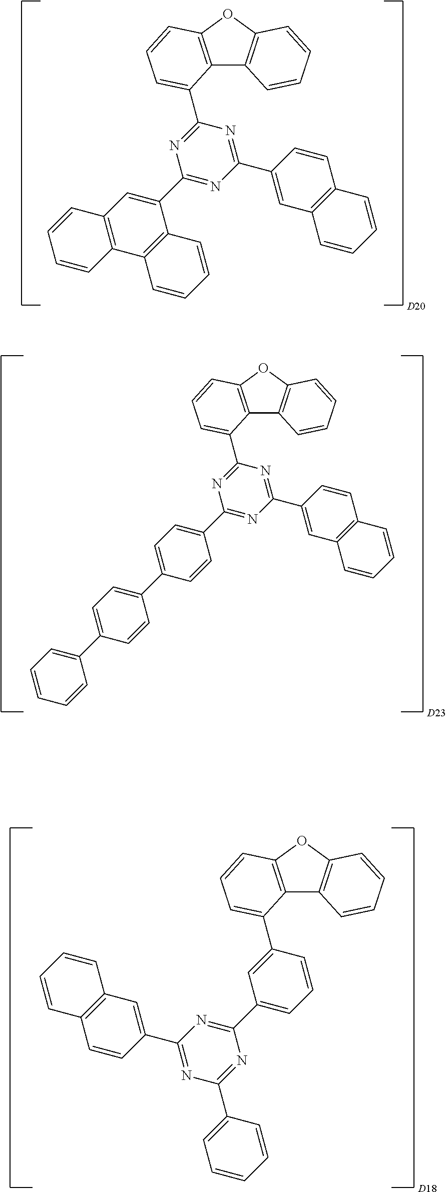

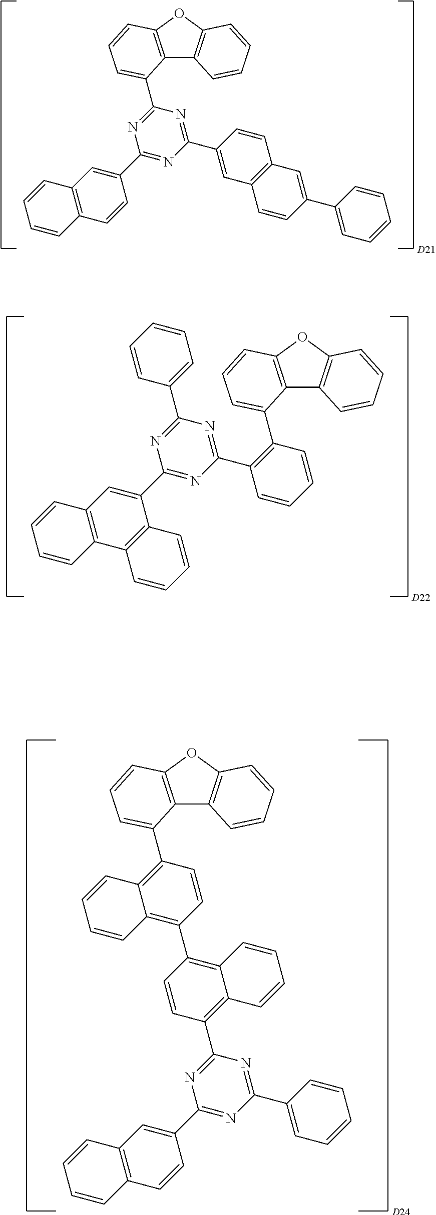

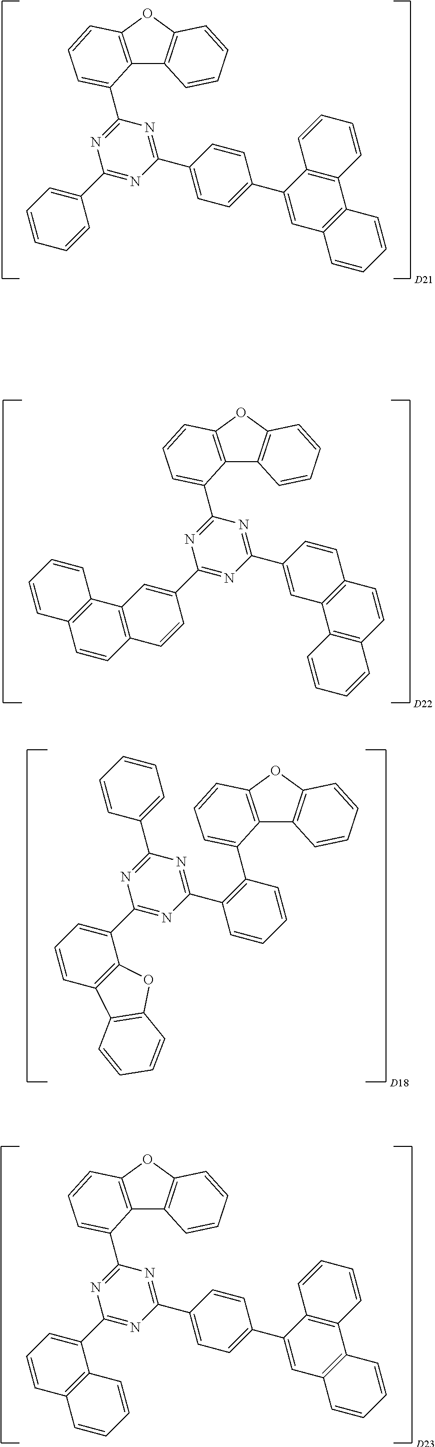

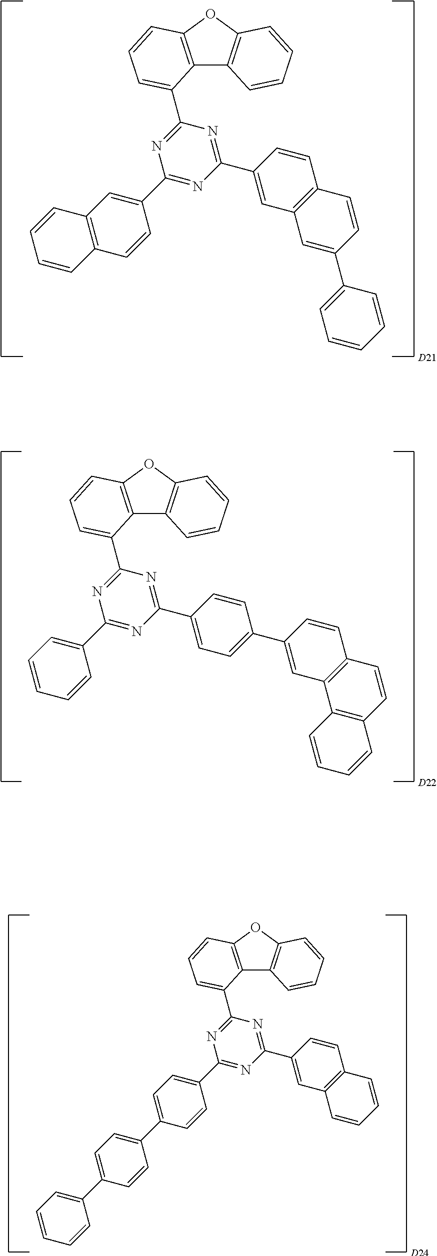

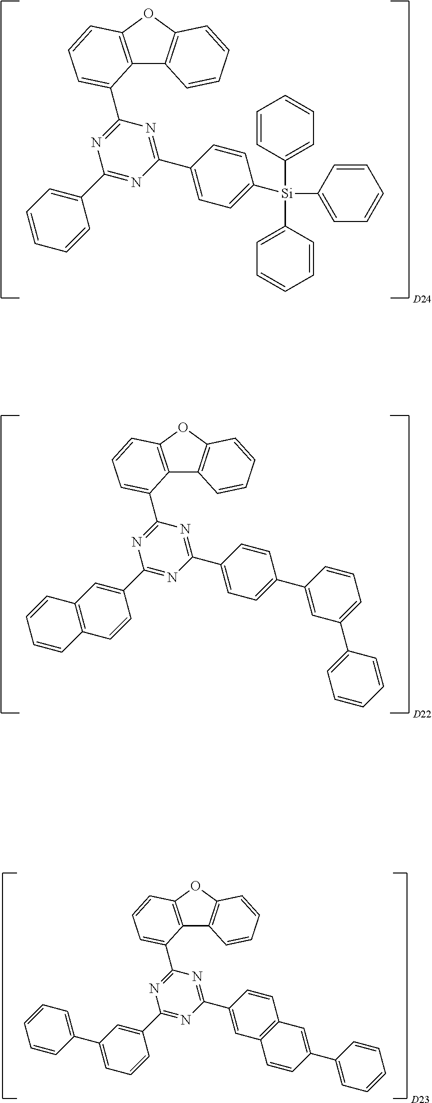

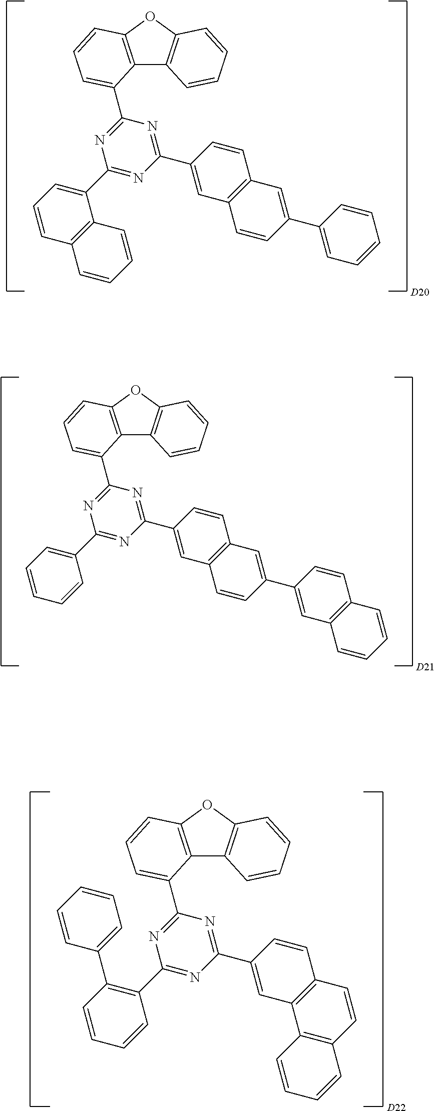

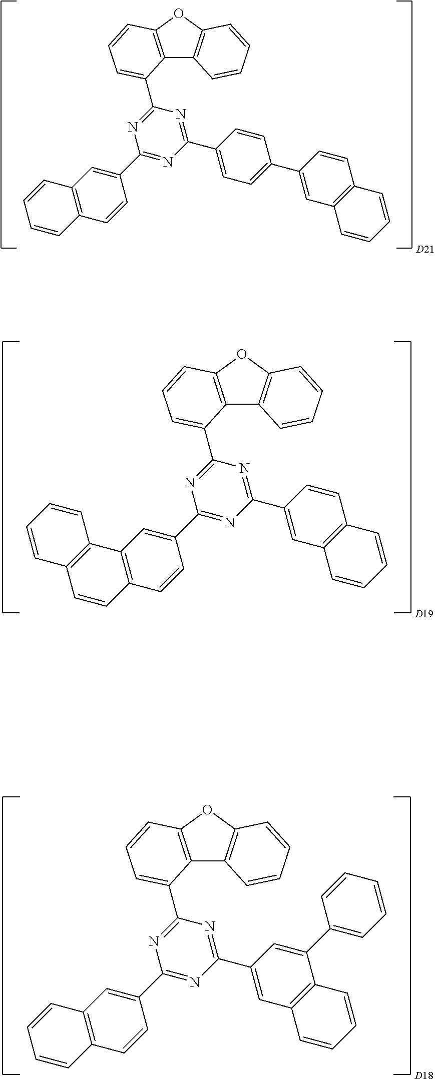

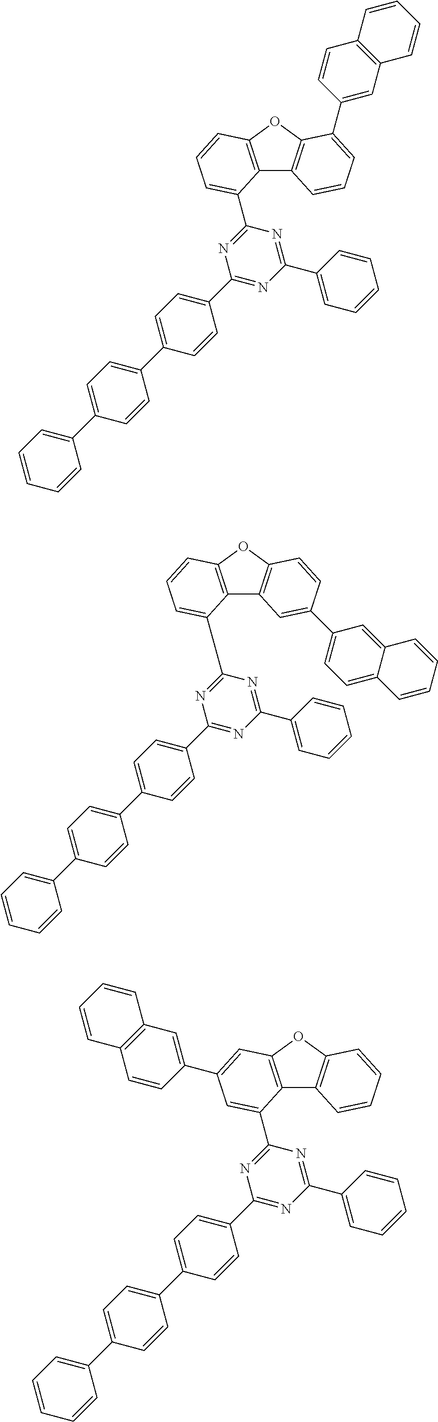 [SMILES: c1ccc(-c2ccc(-c3ccc(-c4nc(-c5ccccc5)nc(-c5cccc6oc7c(-c8ccc9ccccc9c8)cccc7c56)n4)cc3)cc2)cc1 ; c1ccc(-c2ccc(-c3ccc(-c4nc(-c5ccccc5)nc(-c5cccc6oc7ccc(-c8ccc9ccccc9c8)cc7c56)n4)cc3)cc2)cc1 ; c1ccc(-c2ccc(-c3ccc(-c4nc(-c5ccccc5)nc(-c5cc(-c6ccc7ccccc7c6)cc6oc7ccccc7c56)n4)cc3)cc2)cc1]

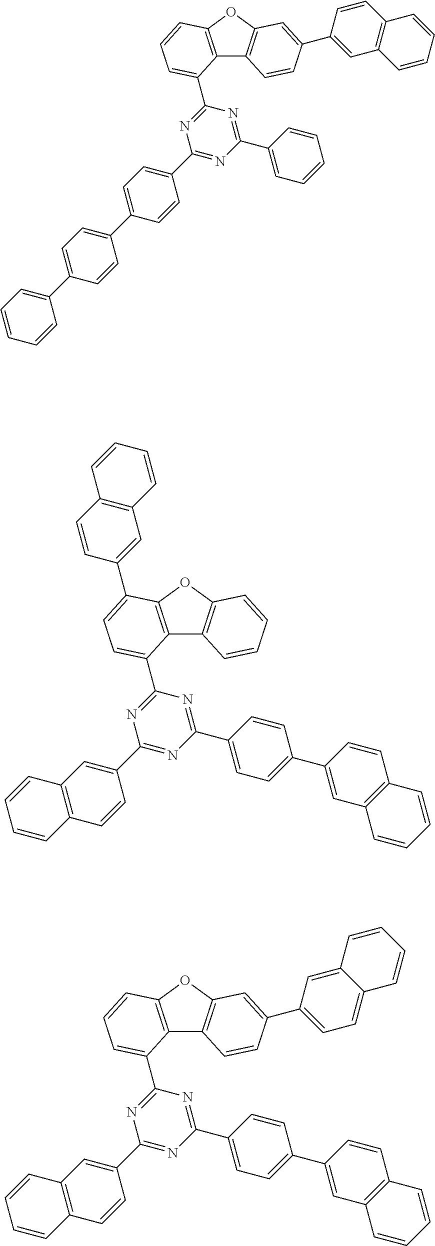 [SMILES: c1ccc(-c2ccc(-c3ccc(-c4nc(-c5ccccc5)nc(-c5cccc6oc7cc(-c8ccc9ccccc9c8)ccc7c56)n4)cc3)cc2)cc1 ; c1ccc2cc(-c3ccc(-c4nc(-c5ccc6ccccc6c5)nc(-c5ccc(-c6ccc7ccccc7c6)c6oc7ccccc7c56)n4)cc3)ccc2c1 ; c1ccc2cc(-c3ccc(-c4nc(-c5ccc6ccccc6c5)nc(-c5cccc6oc7cc(-c8ccc9ccccc9c8)ccc7c56)n4)cc3)ccc2c1]

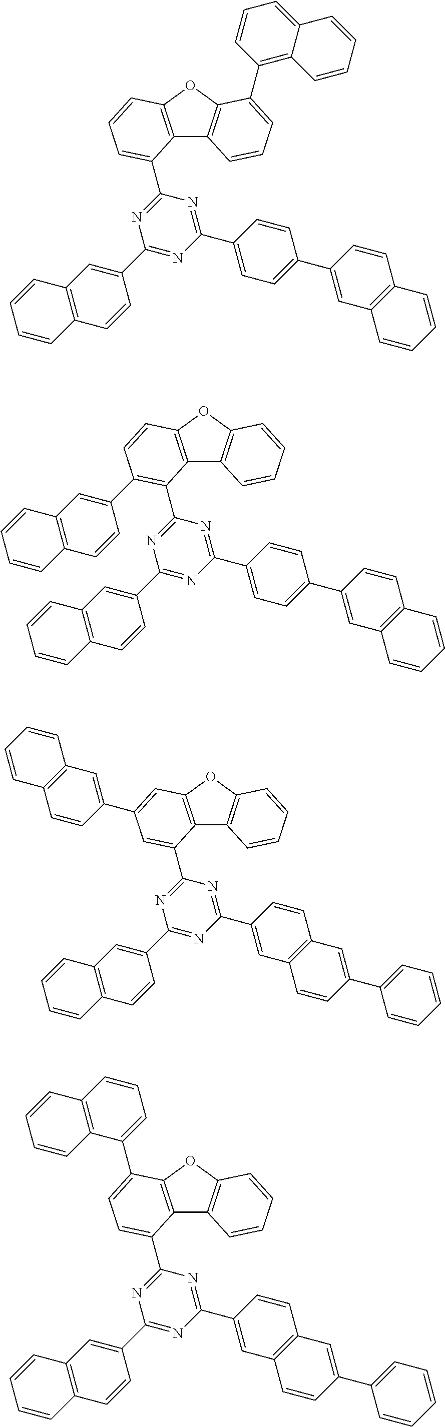 [SMILES: c1ccc2cc(-c3ccc(-c4nc(-c5ccc6ccccc6c5)nc(-c5cccc6oc7c(-c8cccc9ccccc89)cccc7c56)n4)cc3)ccc2c1 ; c1ccc2cc(-c3ccc(-c4nc(-c5ccc6ccccc6c5)nc(-c5c(-c6ccc7ccccc7c6)ccc6oc7ccccc7c56)n4)cc3)ccc2c1 ; c1ccc(-c2ccc3cc(-c4nc(-c5ccc6ccccc6c5)nc(-c5cc(-c6ccc7ccccc7c6)cc6oc7ccccc7c56)n4)ccc3c2)cc1 ; c1ccc(-c2ccc3cc(-c4nc(-c5ccc6ccccc6c5)nc(-c5ccc(-c6cccc7ccccc67)c6oc7ccccc7c56)n4)ccc3c2)cc1]

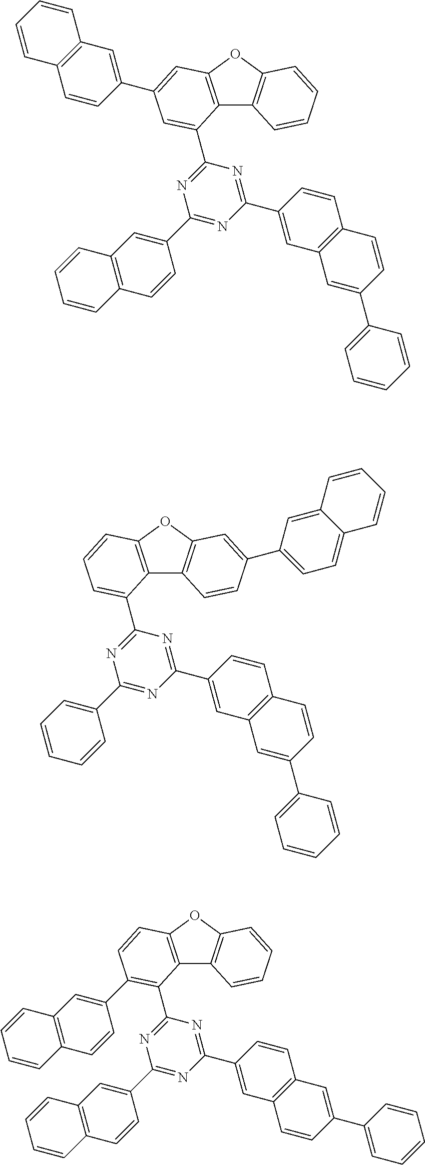 [SMILES: c1ccc(-c2ccc3ccc(-c4nc(-c5ccc6ccccc6c5)nc(-c5cc(-c6ccc7ccccc7c6)cc6oc7ccccc7c56)n4)cc3c2)cc1 ; c1ccc(-c2ccc3ccc(-c4nc(-c5ccccc5)nc(-c5cccc6oc7cc(-c8ccc9ccccc9c8)ccc7c56)n4)cc3c2)cc1 ; c1ccc(-c2ccc3cc(-c4nc(-c5ccc6ccccc6c5)nc(-c5c(-c6ccc7ccccc7c6)ccc6oc7ccccc7c56)n4)ccc3c2)cc1]

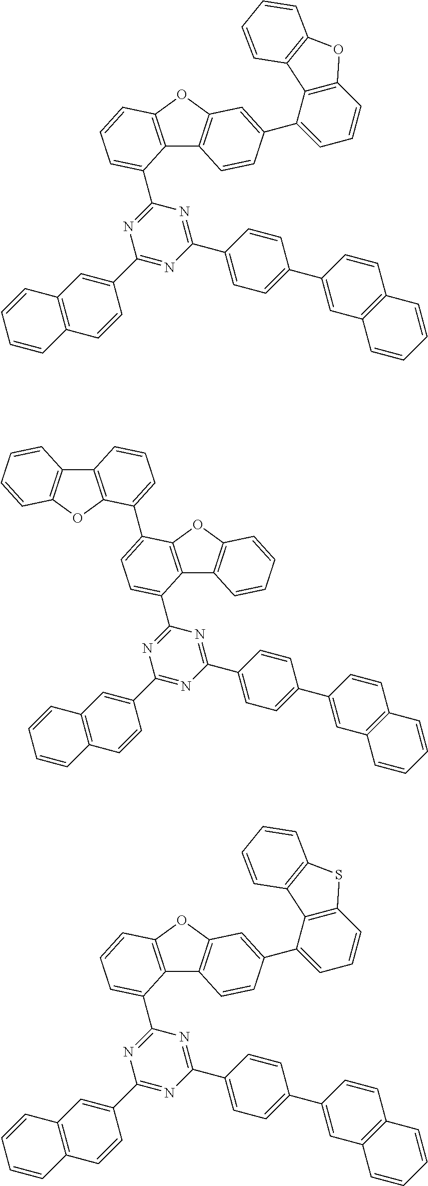 [SMILES: c1ccc2cc(-c3ccc(-c4nc(-c5ccc6ccccc6c5)nc(-c5cccc6oc7cc(-c8cccc9oc%10ccccc%10c89)ccc7c56)n4)cc3)ccc2c1 ; c1ccc2cc(-c3ccc(-c4nc(-c5ccc6ccccc6c5)nc(-c5ccc(-c6cccc7c6oc6ccccc67)c6oc7ccccc7c56)n4)cc3)ccc2c1 ; c1ccc2cc(-c3ccc(-c4nc(-c5ccc6ccccc6c5)nc(-c5cccc6oc7cc(-c8cccc9sc%10ccccc%10c89)ccc7c56)n4)cc3)ccc2c1]

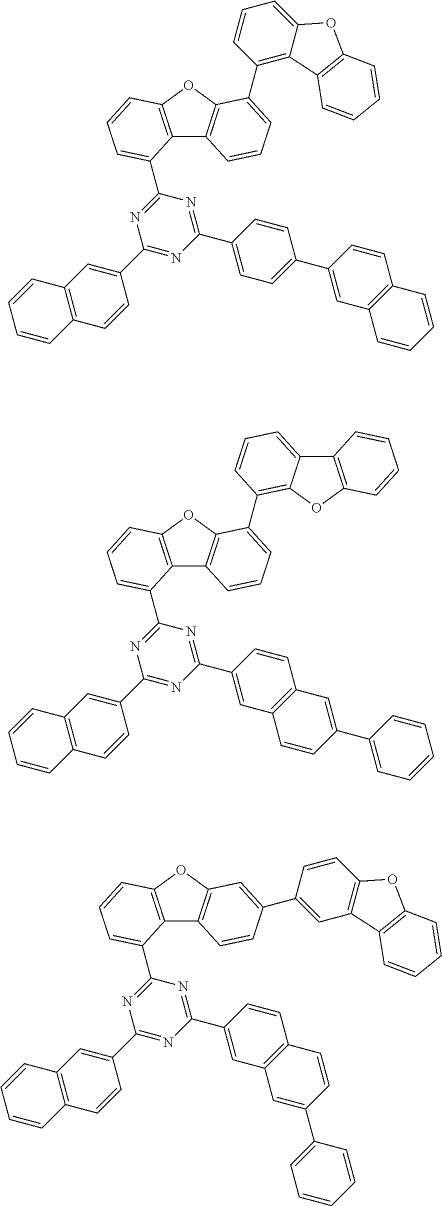 [SMILES: c1ccc2cc(-c3ccc(-c4nc(-c5ccc6ccccc6c5)nc(-c5cccc6oc7c(-c8cccc9oc%10ccccc%10c89)cccc7c56)n4)cc3)ccc2c1 ; c1ccc(-c2ccc3cc(-c4nc(-c5ccc6ccccc6c5)nc(-c5cccc6oc7c(-c8cccc9c8oc8ccccc89)cccc7c56)n4)ccc3c2)cc1 ; c1ccc(-c2ccc3ccc(-c4nc(-c5ccc6ccccc6c5)nc(-c5cccc6oc7cc(-c8ccc9oc%10ccccc%10c9c8)ccc7c56)n4)cc3c2)cc1]

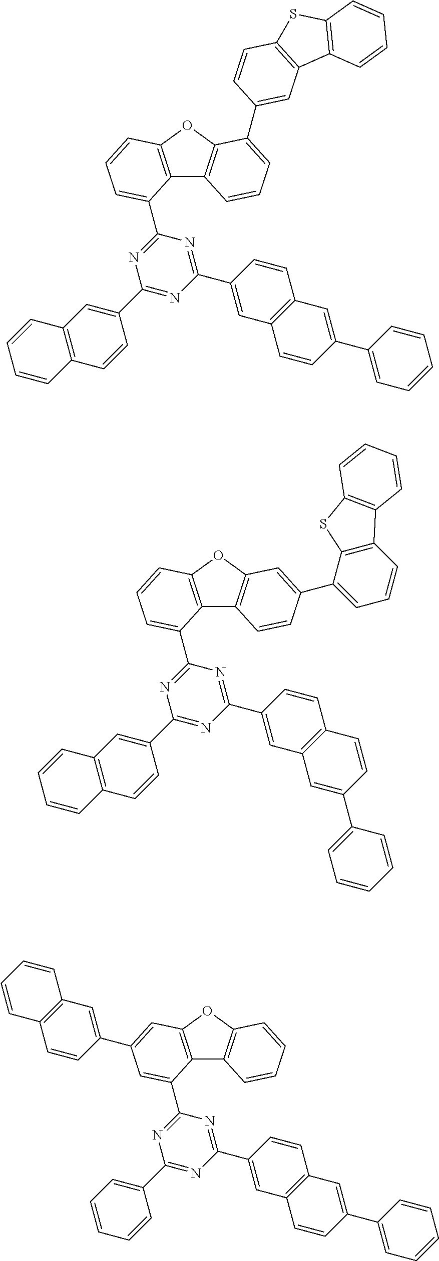 [SMILES: c1ccc(-c2ccc3cc(-c4nc(-c5ccc6ccccc6c5)nc(-c5cccc6oc7c(-c8ccc9sc%10ccccc%10c9c8)cccc7c56)n4)ccc3c2)cc1 ; c1ccc(-c2ccc3ccc(-c4nc(-c5ccc6ccccc6c5)nc(-c5cccc6oc7cc(-c8cccc9c8sc8ccccc89)ccc7c56)n4)cc3c2)cc1 ; c1ccc(-c2ccc3cc(-c4nc(-c5ccccc5)nc(-c5cc(-c6ccc7ccccc7c6)cc6oc7ccccc7c56)n4)ccc3c2)cc1]

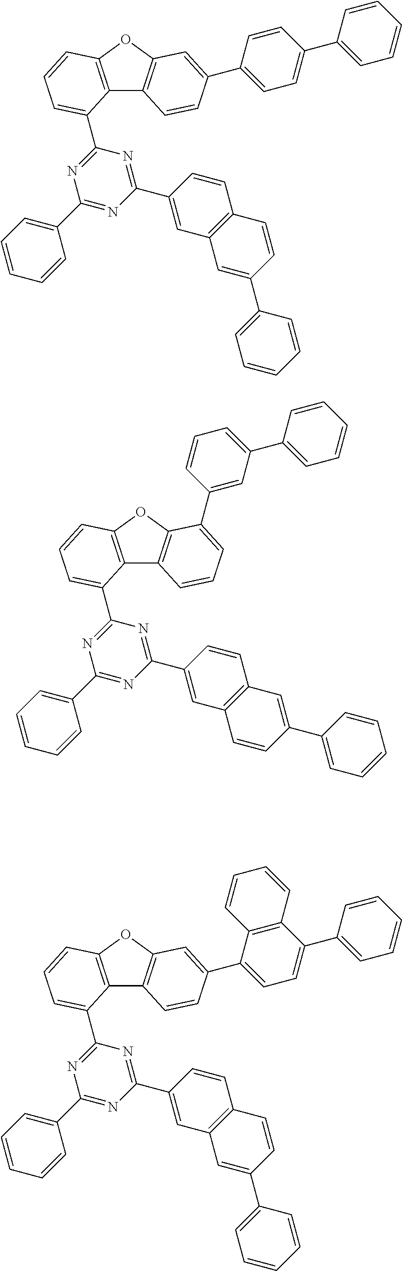

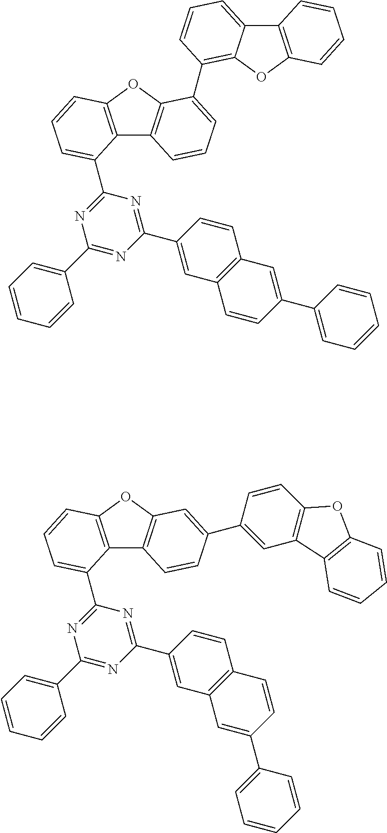 [SMILES: c1ccc(-c2ccc3cc(-c4nc(-c5ccccc5)nc(-c5cccc6oc7c(-c8cccc9c8oc8ccccc89)cccc7c56)n4)ccc3c2)cc1 ; c1ccc(-c2ccc3ccc(-c4nc(-c5ccccc5)nc(-c5cccc6oc7cc(-c8ccc9oc%10ccccc%10c9c8)ccc7c56)n4)cc3c2)cc1]

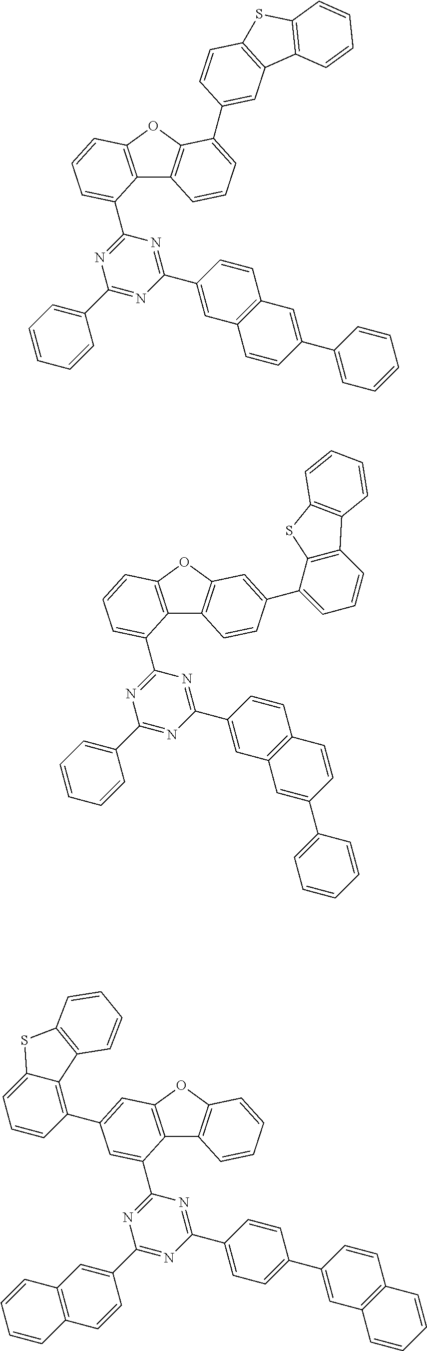 [SMILES: c1ccc(-c2ccc3cc(-c4nc(-c5ccccc5)nc(-c5cccc6oc7c(-c8ccc9sc%10ccccc%10c9c8)cccc7c56)n4)ccc3c2)cc1 ; c1ccc(-c2ccc3ccc(-c4nc(-c5ccccc5)nc(-c5cccc6oc7cc(-c8cccc9c8sc8ccccc89)ccc7c56)n4)cc3c2)cc1 ; c1ccc2cc(-c3ccc(-c4nc(-c5ccc6ccccc6c5)nc(-c5cc(-c6cccc7sc8ccccc8c67)cc6oc7ccccc7c56)n4)cc3)ccc2c1]

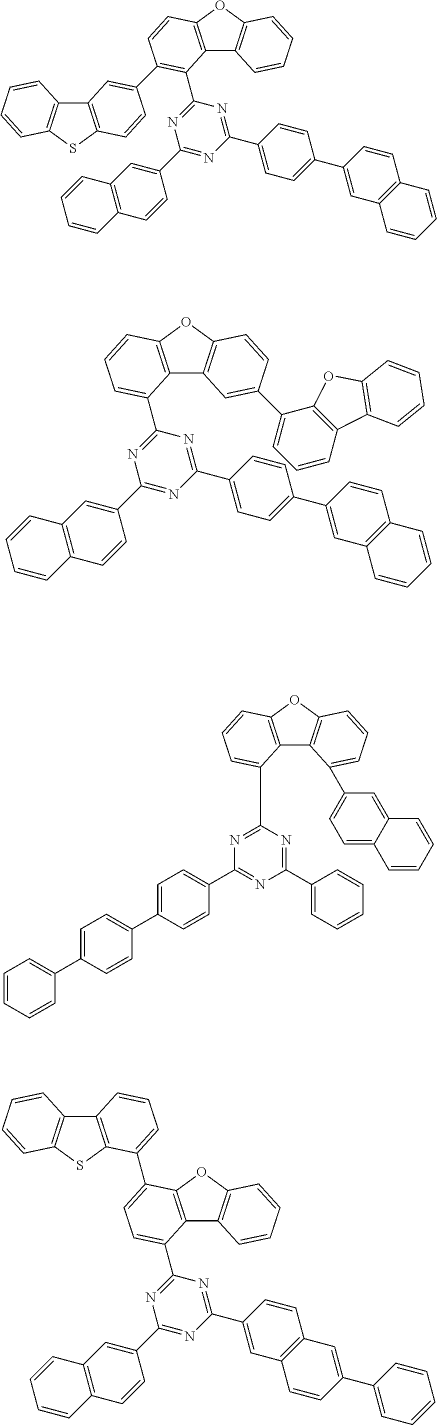 [SMILES: c1ccc2cc(-c3ccc(-c4nc(-c5ccc6ccccc6c5)nc(-c5c(-c6ccc7sc8ccccc8c7c6)ccc6oc7ccccc7c56)n4)cc3)ccc2c1 ; c1ccc2cc(-c3ccc(-c4nc(-c5ccc6ccccc6c5)nc(-c5cccc6oc7ccc(-c8cccc9c8oc8ccccc89)cc7c56)n4)cc3)ccc2c1 ; c1ccc(-c2ccc(-c3ccc(-c4nc(-c5ccccc5)nc(-c5cccc6oc7cccc(-c8ccc9ccccc9c8)c7c56)n4)cc3)cc2)cc1 ; c1ccc(-c2ccc3cc(-c4nc(-c5ccc6ccccc6c5)nc(-c5ccc(-c6cccc7c6sc6ccccc67)c6oc7ccccc7c56)n4)ccc3c2)cc1]

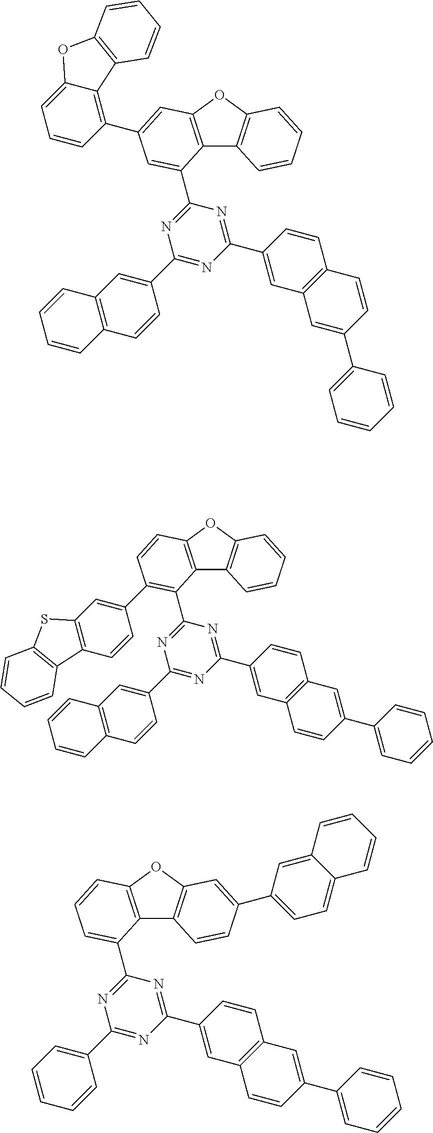 [SMILES: c1ccc(-c2ccc3ccc(-c4nc(-c5ccc6ccccc6c5)nc(-c5cc(-c6cccc7oc8ccccc8c67)cc6oc7ccccc7c56)n4)cc3c2)cc1 ; c1ccc(-c2ccc3cc(-c4nc(-c5ccc6ccccc6c5)nc(-c5c(-c6ccc7c(c6)sc6ccccc67)ccc6oc7ccccc7c56)n4)ccc3c2)cc1 ; c1ccc(-c2ccc3cc(-c4nc(-c5ccccc5)nc(-c5cccc6oc7cc(-c8ccc9ccccc9c8)ccc7c56)n4)ccc3c2)cc1]

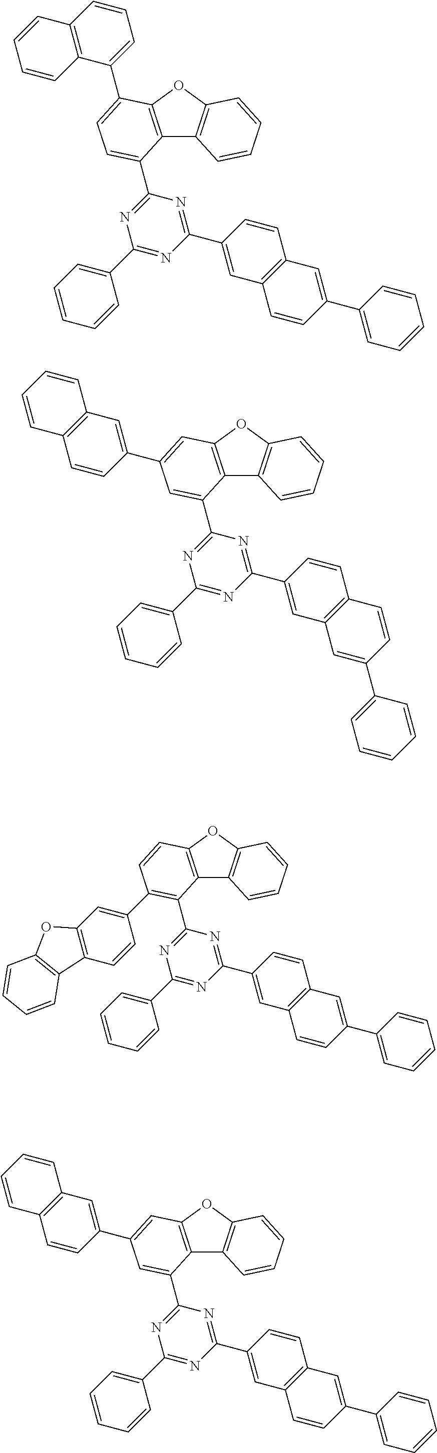 [SMILES: c1ccc(-c2ccc3cc(-c4nc(-c5ccccc5)nc(-c5ccc(-c6cccc7ccccc67)c6oc7ccccc7c56)n4)ccc3c2)cc1 ; c1ccc(-c2ccc3ccc(-c4nc(-c5ccccc5)nc(-c5cc(-c6ccc7ccccc7c6)cc6oc7ccccc7c56)n4)cc3c2)cc1 ; c1ccc(-c2ccc3cc(-c4nc(-c5ccccc5)nc(-c5c(-c6ccc7c(c6)oc6ccccc67)ccc6oc7ccccc7c56)n4)ccc3c2)cc1 ; c1ccc(-c2ccc3cc(-c4nc(-c5ccccc5)nc(-c5cc(-c6ccc7ccccc7c6)cc6oc7ccccc7c56)n4)ccc3c2)cc1]

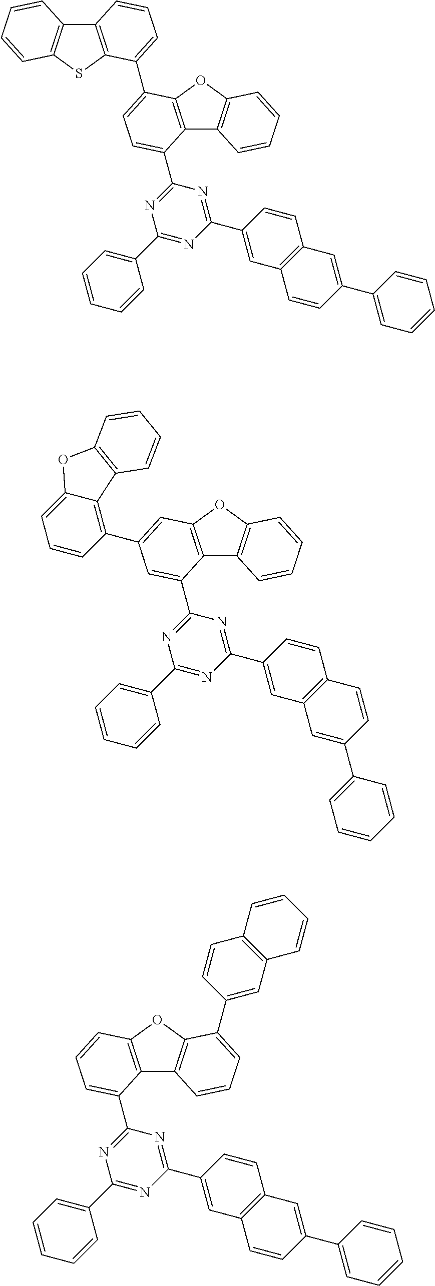 [SMILES: c1ccc(-c2ccc3cc(-c4nc(-c5ccccc5)nc(-c5ccc(-c6cccc7c6sc6ccccc67)c6oc7ccccc7c56)n4)ccc3c2)cc1 ; c1ccc(-c2ccc3ccc(-c4nc(-c5ccccc5)nc(-c5cc(-c6cccc7oc8ccccc8c67)cc6oc7ccccc7c56)n4)cc3c2)cc1 ; c1ccc(-c2ccc3cc(-c4nc(-c5ccccc5)nc(-c5cccc6oc7c(-c8ccc9ccccc9c8)cccc7c56)n4)ccc3c2)cc1]

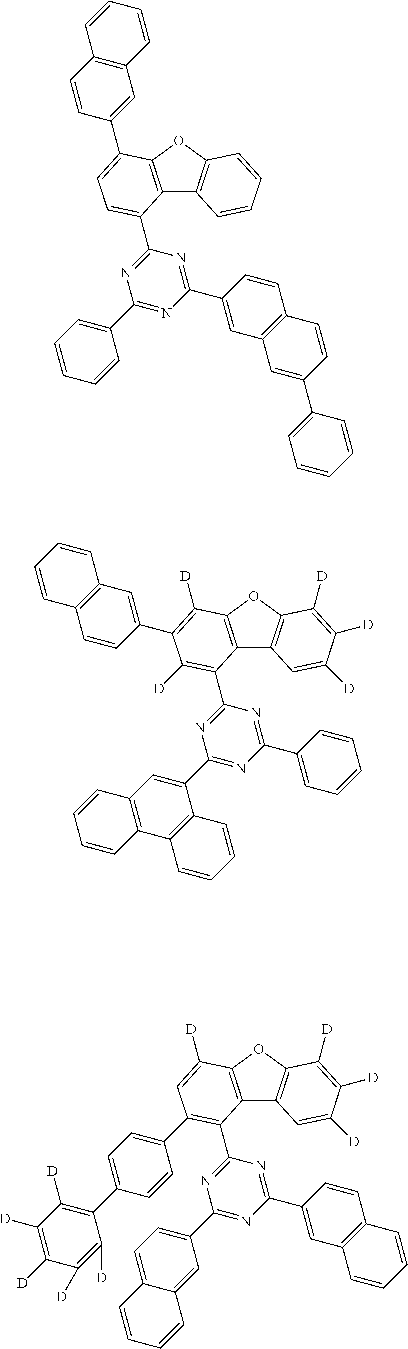 [SMILES: c1ccc(-c2ccc3ccc(-c4nc(-c5ccccc5)nc(-c5ccc(-c6ccc7ccccc7c6)c6oc7ccccc7c56)n4)cc3c2)cc1 ; [2H]c1cc2c(oc3c([2H])c(-c4ccc5ccccc5c4)c([2H])c(-c4nc(-c5ccccc5)nc(-c5cc6ccccc6c6ccccc56)n4)c32)c([2H])c1[2H] ; [2H]c1cc2c(oc3c([2H])cc(-c4ccc(-c5c([2H])c([2H])c([2H])c([2H])c5[2H])cc4)c(-c4nc(-c5ccc6ccccc6c5)nc(-c5ccc6ccccc6c5)n4)c32)c([2H])c1[2H]]

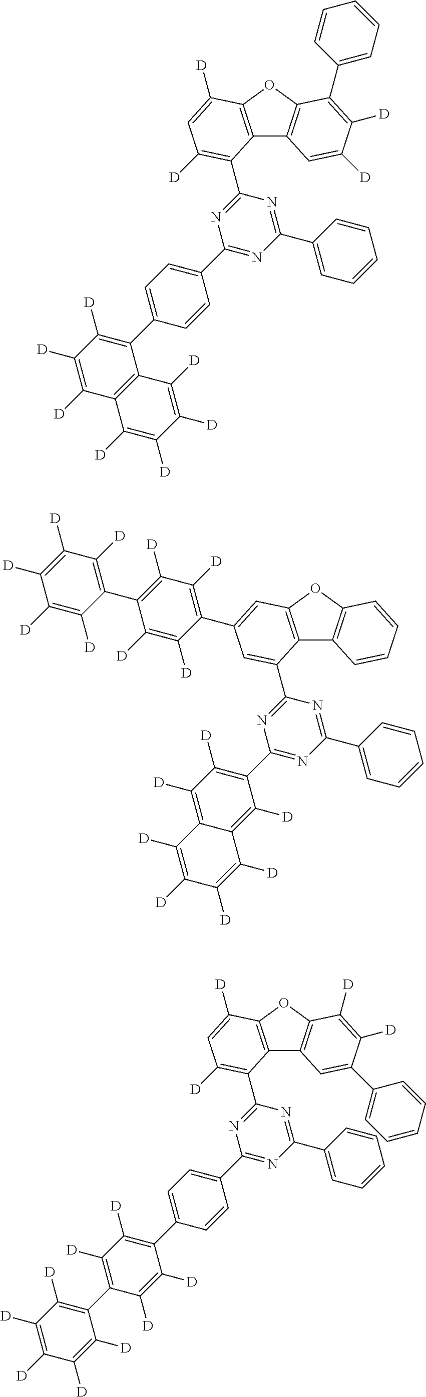 [SMILES: [2H]c1cc2c(oc3c([2H])cc([2H])c(-c4nc(-c5ccccc5)nc(-c5ccc(-c6c([2H])c([2H])c([2H])c7c([2H])c([2H])c([2H])c([2H])c67)cc5)n4)c32)c(-c2ccccc2)c1[2H] ; [2H]c1c([2H])c([2H])c(-c2c([2H])c([2H])c(-c3cc(-c4nc(-c5ccccc5)nc(-c5c([2H])c([2H])c6c([2H])c([2H])c([2H])c([2H])c6c5[2H])n4)c4c(c3)oc3ccccc34)c([2H])c2[2H])c([2H])c1[2H] ; [2H]c1cc([2H])c2oc3c([2H])c([2H])c(-c4ccccc4)cc3c2c1-c1nc(-c2ccccc2)nc(-c2ccc(-c3c([2H])c([2H])c(-c4c([2H])c([2H])c([2H])c([2H])c4[2H])c([2H])c3[2H])cc2)n1]

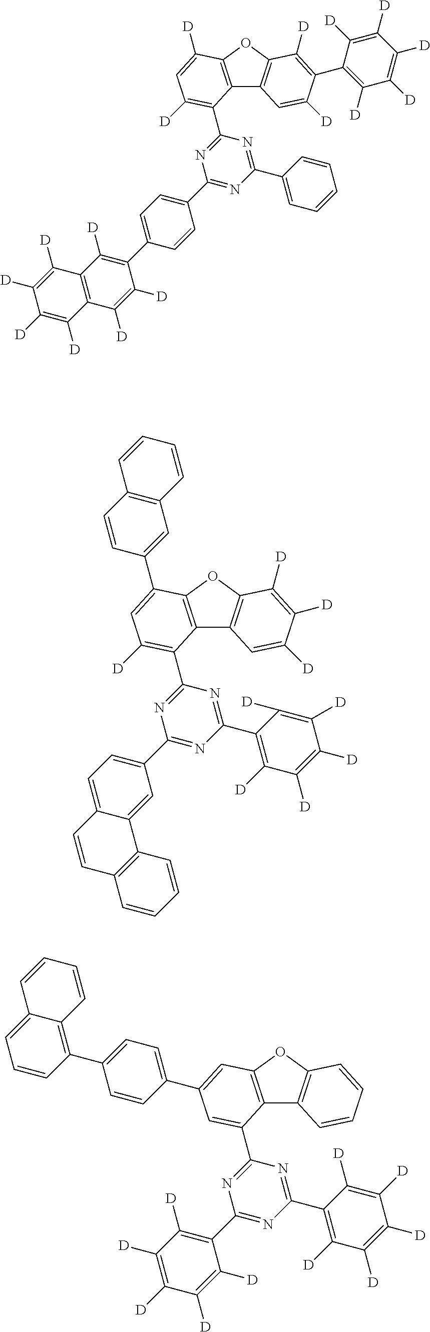 [SMILES: [2H]c1cc2c(oc3c([2H])cc([2H])c(-c4nc(-c5ccccc5)nc(-c5ccc(-c6c([2H])c([2H])c7c([2H])c([2H])c([2H])c([2H])c7c6[2H])cc5)n4)c32)c([2H])c1-c1c([2H])c([2H])c([2H])c([2H])c1[2H] ; [2H]c1cc2c(oc3c(-c4ccc5ccccc5c4)cc([2H])c(-c4nc(-c5ccc6ccc7ccccc7c6c5)nc(-c5c([2H])c([2H])c([2H])c([2H])c5[2H])n4)c32)c([2H])c1[2H] ; [2H]c1c([2H])c([2H])c(-c2nc(-c3c([2H])c([2H])c([2H])c([2H])c3[2H])nc(-c3cc(-c4ccc(-c5cccc6ccccc56)cc4)cc4oc5ccccc5c34)n2)c([2H])c1[2H]]

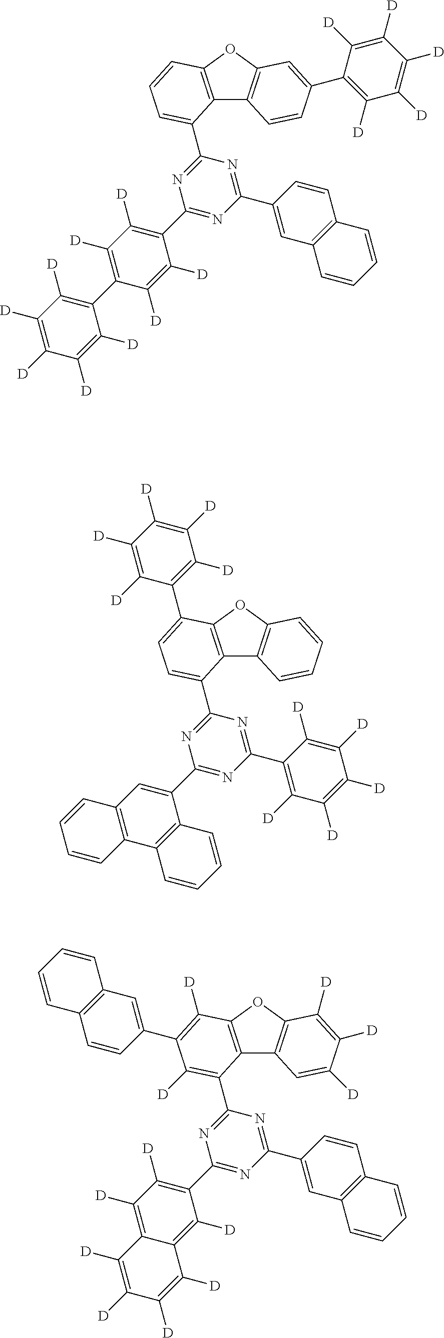 [SMILES: [2H]c1c([2H])c([2H])c(-c2ccc3c(c2)oc2cccc(-c4nc(-c5ccc6ccccc6c5)nc(-c5c([2H])c([2H])c(-c6c([2H])c([2H])c([2H])c([2H])c6[2H])c([2H])c5[2H])n4)c23)c([2H])c1[2H] ; [2H]c1c([2H])c([2H])c(-c2nc(-c3cc4ccccc4c4ccccc34)nc(-c3ccc(-c4c([2H])c([2H])c([2H])c([2H])c4[2H])c4oc5ccccc5c34)n2)c([2H])c1[2H] ; [2H]c1cc2c(oc3c([2H])c(-c4ccc5ccccc5c4)c([2H])c(-c4nc(-c5ccc6ccccc6c5)nc(-c5c([2H])c([2H])c6c([2H])c([2H])c([2H])c([2H])c6c5[2H])n4)c32)c([2H])c1[2H]]

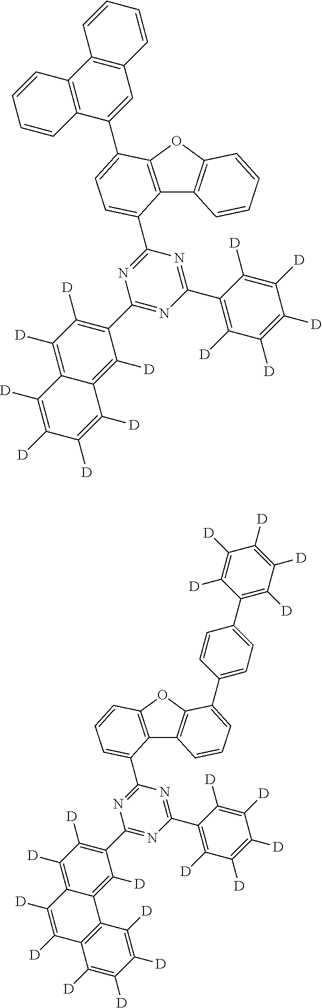 [SMILES: [2H]c1c([2H])c([2H])c(-c2nc(-c3c([2H])c([2H])c4c([2H])c([2H])c([2H])c([2H])c4c3[2H])nc(-c3ccc(-c4cc5ccccc5c5ccccc45)c4oc5ccccc5c34)n2)c([2H])c1[2H] ; [2H]c1c([2H])c([2H])c(-c2ccc(-c3cccc4c3oc3cccc(-c5nc(-c6c([2H])c([2H])c([2H])c([2H])c6[2H])nc(-c6c([2H])c([2H])c7c([2H])c([2H])c8c([2H])c([2H])c([2H])c([2H])c8c7c6[2H])n5)c34)cc2)c([2H])c1[2H]]

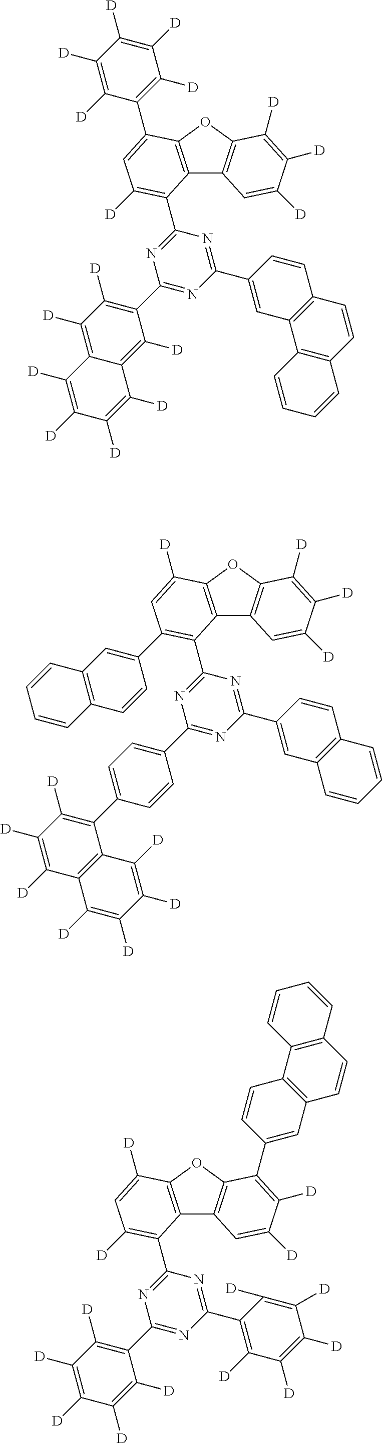 [SMILES: [2H]c1cc2c(oc3c(-c4c([2H])c([2H])c([2H])c([2H])c4[2H])cc([2H])c(-c4nc(-c5ccc6ccc7ccccc7c6c5)nc(-c5c([2H])c([2H])c6c([2H])c([2H])c([2H])c([2H])c6c5[2H])n4)c32)c([2H])c1[2H] ; [2H]c1cc2c(oc3c([2H])cc(-c4ccc5ccccc5c4)c(-c4nc(-c5ccc(-c6c([2H])c([2H])c([2H])c7c([2H])c([2H])c([2H])c([2H])c67)cc5)nc(-c5ccc6ccccc6c5)n4)c32)c([2H])c1[2H] ; [2H]c1cc2c(oc3c([2H])cc([2H])c(-c4nc(-c5c([2H])c([2H])c([2H])c([2H])c5[2H])nc(-c5c([2H])c([2H])c([2H])c([2H])c5[2H])n4)c32)c(-c2ccc3c(ccc4ccccc43)c2)c1[2H]]

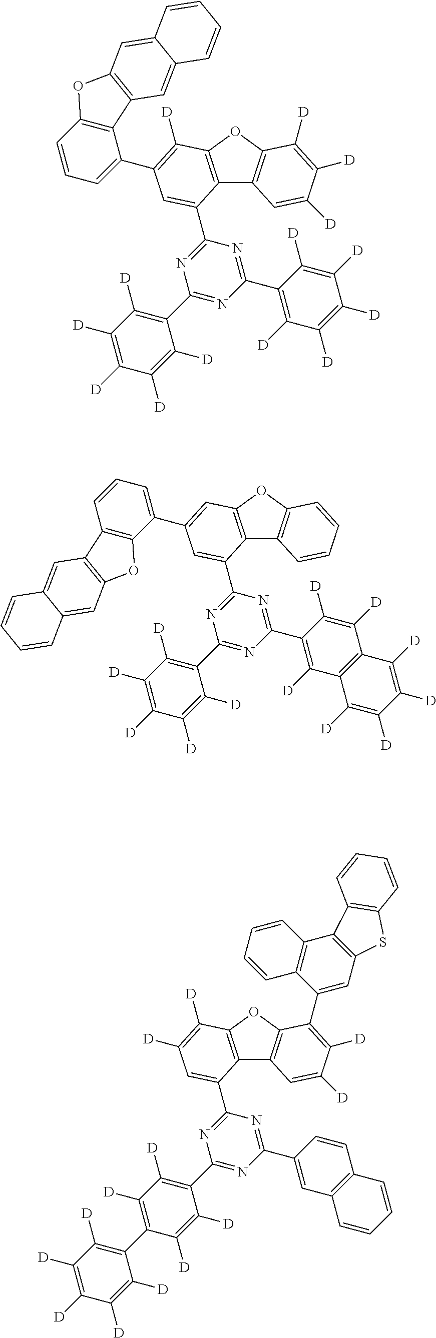 [SMILES: [2H]c1cc2c(oc3c([2H])c(-c4cccc5oc6cc7ccccc7cc6c45)cc(-c4nc(-c5c([2H])c([2H])c([2H])c([2H])c5[2H])nc(-c5c([2H])c([2H])c([2H])c([2H])c5[2H])n4)c32)c([2H])c1[2H] ; [2H]c1c([2H])c([2H])c(-c2nc(-c3c([2H])c([2H])c4c([2H])c([2H])c([2H])c([2H])c4c3[2H])nc(-c3cc(-c4cccc5c4oc4cc6ccccc6cc45)cc4oc5ccccc5c34)n2)c([2H])c1[2H] ; [2H]c1cc2c(oc3c([2H])c([2H])cc(-c4nc(-c5ccc6ccccc6c5)nc(-c5c([2H])c([2H])c(-c6c([2H])c([2H])c([2H])c([2H])c6[2H])c([2H])c5[2H])n4)c32)c(-c2cc3sc4ccccc4c3c3ccccc23)c1[2H]]

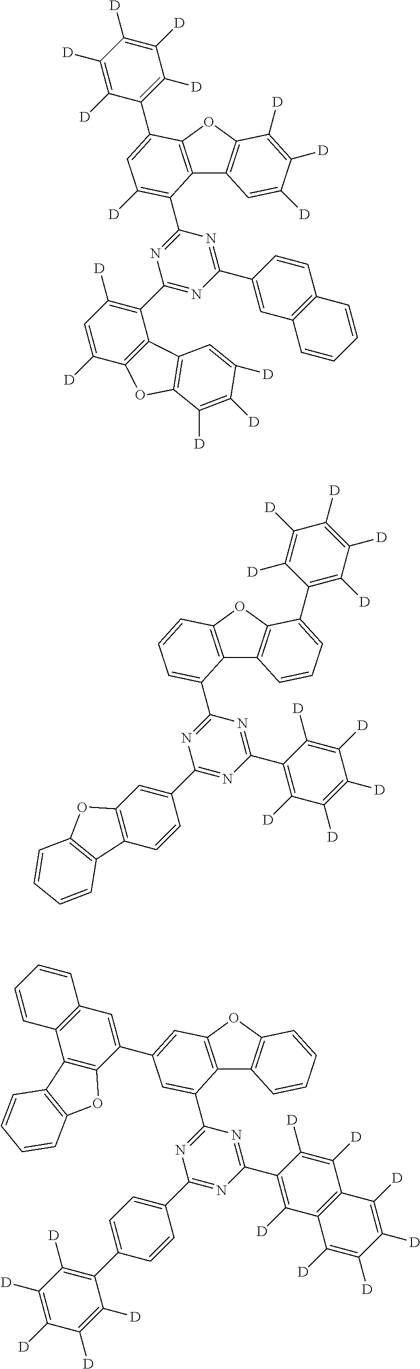 [SMILES: [2H]c1cc2c(oc3c([2H])cc([2H])c(-c4nc(-c5ccc6ccccc6c5)nc(-c5c([2H])cc(-c6c([2H])c([2H])c([2H])c([2H])c6[2H])c6oc7c([2H])c([2H])c([2H])cc7c56)n4)c32)c([2H])c1[2H] ; [2H]c1c([2H])c([2H])c(-c2nc(-c3ccc4c(c3)oc3ccccc34)nc(-c3cccc4oc5c(-c6c([2H])c([2H])c([2H])c([2H])c6[2H])cccc5c34)n2)c([2H])c1[2H] ; [2H]c1c([2H])c([2H])c(-c2ccc(-c3nc(-c4c([2H])c([2H])c5c([2H])c([2H])c([2H])c([2H])c5c4[2H])nc(-c4cc(-c5cc6ccccc6c6c5oc5ccccc56)cc5oc6ccccc6c45)n3)cc2)c([2H])c1[2H]]

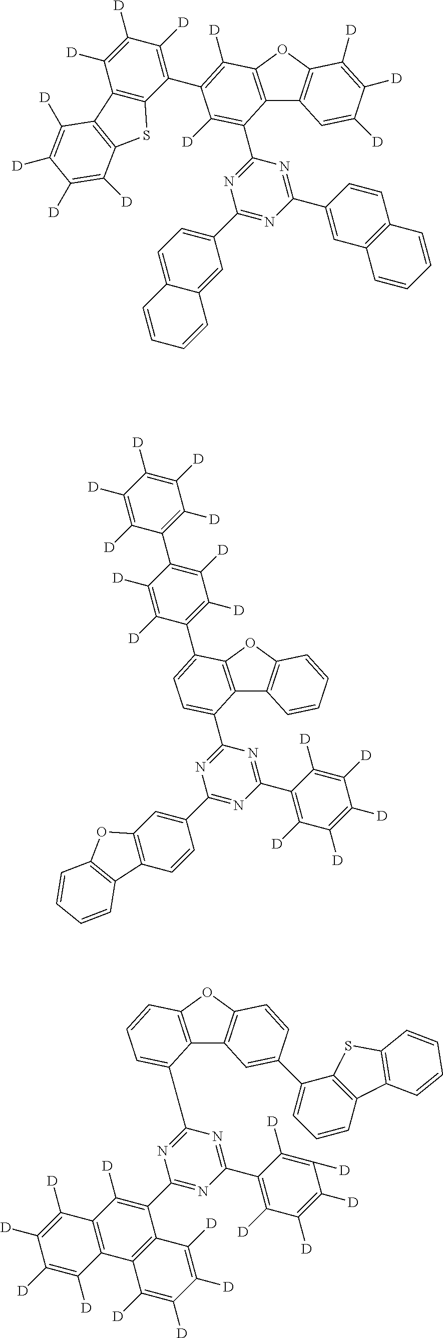 [SMILES: [2H]c1cc2c(oc3c([2H])c(-c4c([2H])c([2H])c([2H])c5c4sc4c([2H])c([2H])c([2H])c([2H])c45)c([2H])c(-c4nc(-c5ccc6ccccc6c5)nc(-c5ccc6ccccc6c5)n4)c32)c([2H])c1[2H] ; [2H]c1c([2H])c([2H])c(-c2nc(-c3ccc4c(c3)oc3ccccc34)nc(-c3ccc(-c4c([2H])c([2H])c(-c5c([2H])c([2H])c([2H])c([2H])c5[2H])c([2H])c4[2H])c4oc5ccccc5c34)n2)c([2H])c1[2H] ; [2H]c1c([2H])c([2H])c(-c2nc(-c3c([2H])c4c([2H])c([2H])c([2H])c([2H])c4c4c([2H])c([2H])c([2H])c([2H])c34)nc(-c3cccc4oc5ccc(-c6cccc7c6sc6ccccc67)cc5c34)n2)c([2H])c1[2H]]

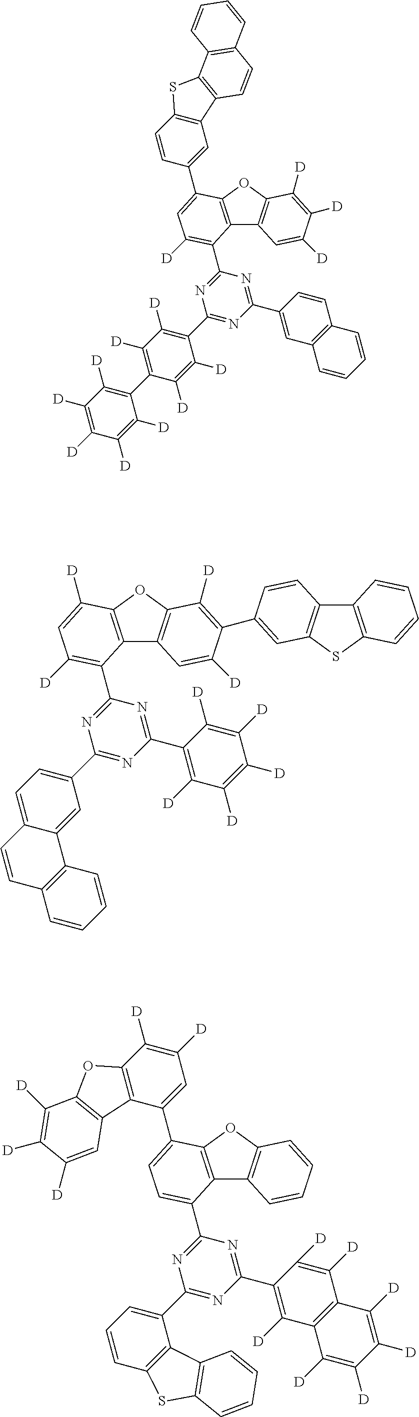 [SMILES: [2H]c1cc2c(oc3c(-c4ccc5sc6c7ccccc7ccc6c5c4)cc([2H])c(-c4nc(-c5ccc6ccccc6c5)nc(-c5c([2H])c([2H])c(-c6c([2H])c([2H])c([2H])c([2H])c6[2H])c([2H])c5[2H])n4)c32)c([2H])c1[2H] ; [2H]c1cc2c(oc3c([2H])cc([2H])c(-c4nc(-c5ccc6ccc7ccccc7c6c5)nc(-c5c([2H])c([2H])c([2H])c([2H])c5[2H])n4)c32)c([2H])c1-c1ccc2c(c1)sc1ccccc12 ; [2H]c1cc2c(oc3c([2H])c([2H])cc(-c4ccc(-c5nc(-c6c([2H])c([2H])c7c([2H])c([2H])c([2H])c([2H])c7c6[2H])nc(-c6cccc7sc8ccccc8c67)n5)c5c4oc4ccccc45)c32)c([2H])c1[2H]]

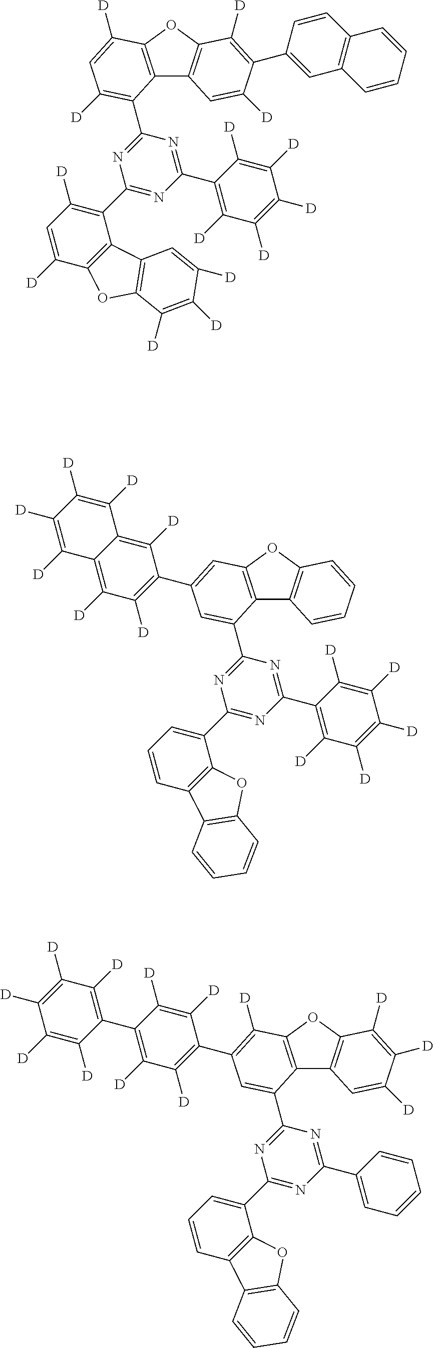 [SMILES: [2H]c1cc2c(oc3c([2H])cc([2H])c(-c4nc(-c5c([2H])c([2H])c([2H])c([2H])c5[2H])nc(-c5c([2H])cc([2H])c6oc7c([2H])c(-c8ccc9ccccc9c8)c([2H])cc7c56)n4)c32)c([2H])c1[2H] ; [2H]c1c([2H])c([2H])c(-c2nc(-c3cccc4c3oc3ccccc34)nc(-c3cc(-c4c([2H])c([2H])c5c([2H])c([2H])c([2H])c([2H])c5c4[2H])cc4oc5ccccc5c34)n2)c([2H])c1[2H] ; [2H]c1cc2c(oc3c([2H])c(-c4c([2H])c([2H])c(-c5c([2H])c([2H])c([2H])c([2H])c5[2H])c([2H])c4[2H])cc(-c4nc(-c5ccccc5)nc(-c5cccc6c5oc5ccccc56)n4)c32)c([2H])c1[2H]]

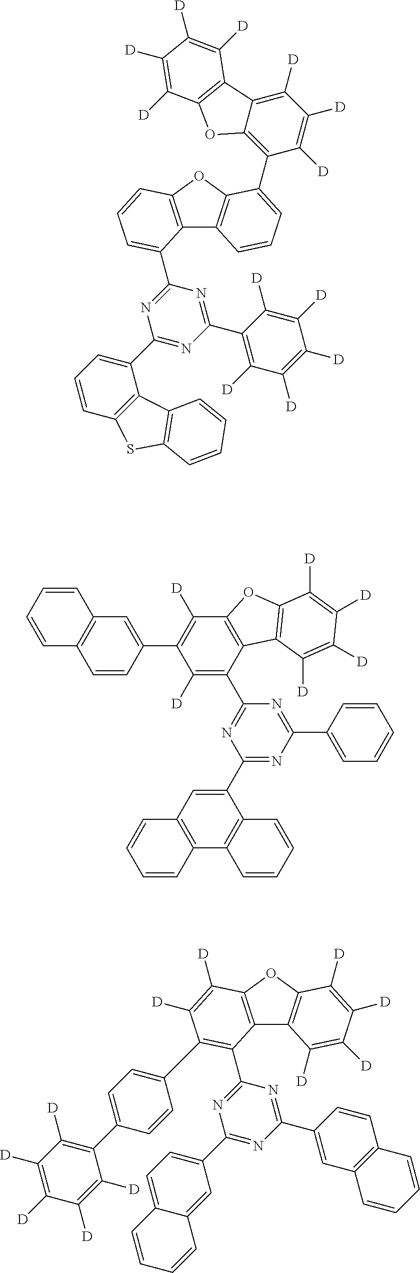 [SMILES: [2H]c1c([2H])c([2H])c(-c2nc(-c3cccc4oc5c(-c6c([2H])c([2H])c([2H])c7c6oc6c([2H])c([2H])c([2H])c([2H])c67)cccc5c34)nc(-c3cccc4sc5ccccc5c34)n2)c([2H])c1[2H] ; [2H]c1c([2H])c([2H])c2c(oc3c([2H])c(-c4ccc5ccccc5c4)c([2H])c(-c4nc(-c5ccccc5)nc(-c5cc6ccccc6c6ccccc56)n4)c32)c1[2H] ; [2H]c1c([2H])c([2H])c(-c2ccc(-c3c([2H])c([2H])c4oc5c([2H])c([2H])c([2H])c([2H])c5c4c3-c3nc(-c4ccc5ccccc5c4)nc(-c4ccc5ccccc5c4)n3)cc2)c([2H])c1[2H]]

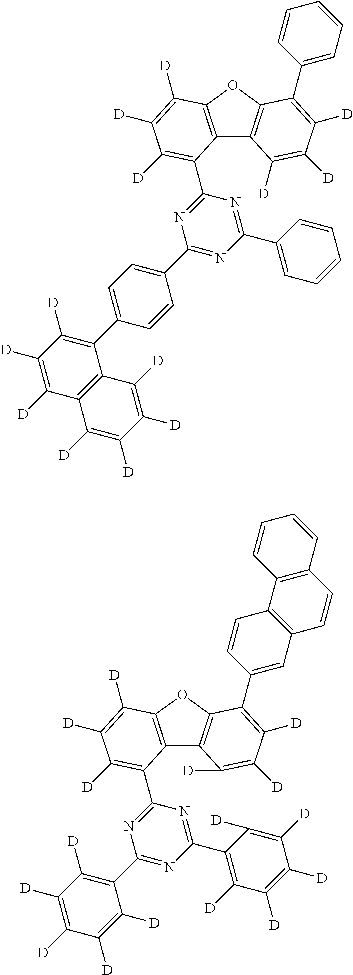 [SMILES: [2H]c1c([2H])c(-c2nc(-c3ccccc3)nc(-c3ccc(-c4c([2H])c([2H])c([2H])c5c([2H])c([2H])c([2H])c([2H])c45)cc3)n2)c2c(oc3c(-c4ccccc4)c([2H])c([2H])c([2H])c32)c1[2H] ; [2H]c1c([2H])c([2H])c(-c2nc(-c3c([2H])c([2H])c([2H])c([2H])c3[2H])nc(-c3c([2H])c([2H])c([2H])c4oc5c(-c6ccc7c(ccc8ccccc87)c6)c([2H])c([2H])c([2H])c5c34)n2)c([2H])c1[2H]]

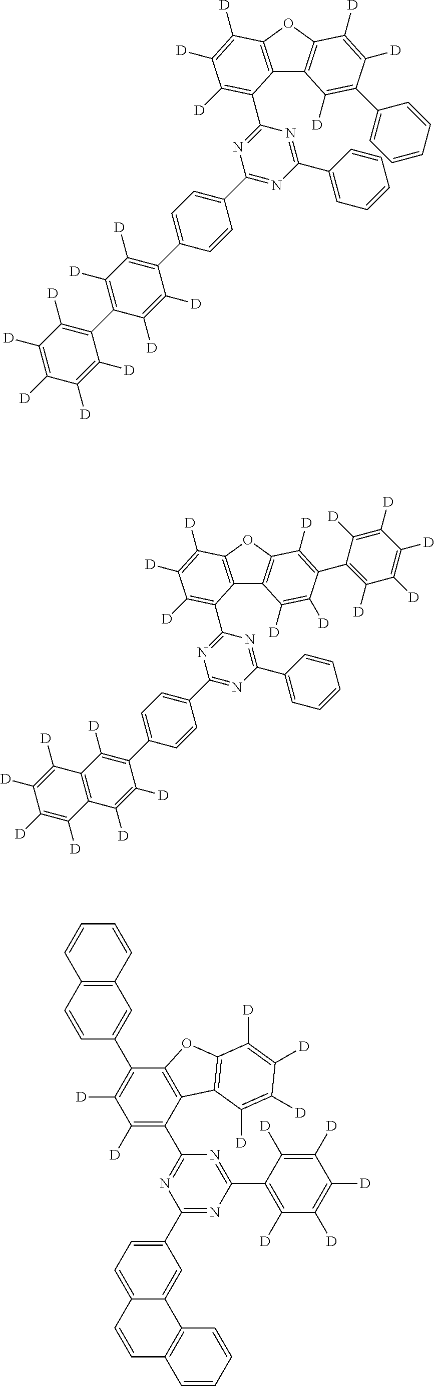 [SMILES: [2H]c1c([2H])c([2H])c(-c2c([2H])c([2H])c(-c3ccc(-c4nc(-c5ccccc5)nc(-c5c([2H])c([2H])c([2H])c6oc7c([2H])c([2H])c(-c8ccccc8)c([2H])c7c56)n4)cc3)c([2H])c2[2H])c([2H])c1[2H] ; [2H]c1c([2H])c([2H])c(-c2c([2H])c([2H])c3c(oc4c([2H])c([2H])c([2H])c(-c5nc(-c6ccccc6)nc(-c6ccc(-c7c([2H])c([2H])c8c([2H])c([2H])c([2H])c([2H])c8c7[2H])cc6)n5)c43)c2[2H])c([2H])c1[2H] ; [2H]c1c([2H])c([2H])c(-c2nc(-c3ccc4ccc5ccccc5c4c3)nc(-c3c([2H])c([2H])c(-c4ccc5ccccc5c4)c4oc5c([2H])c([2H])c([2H])c([2H])c5c34)n2)c([2H])c1[2H]]

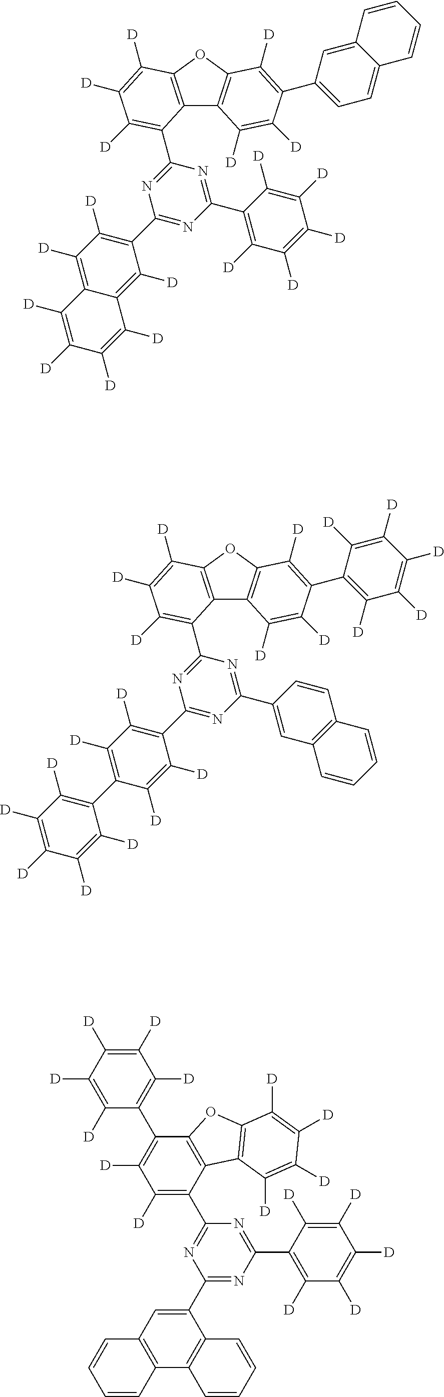

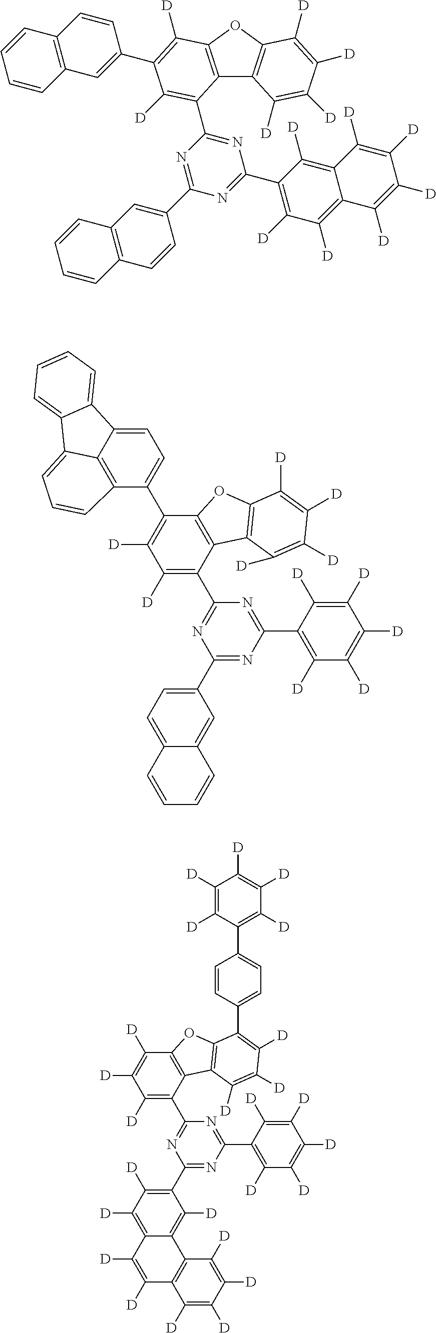

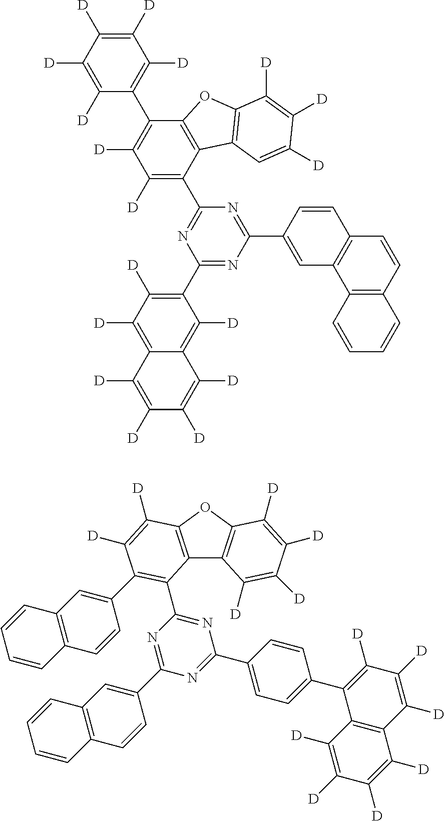 [SMILES: [2H]c1cc2c(oc3c(-c4c([2H])c([2H])c([2H])c([2H])c4[2H])c([2H])c([2H])c(-c4nc(-c5ccc6ccc7ccccc7c6c5)nc(-c5c([2H])c([2H])c6c([2H])c([2H])c([2H])c([2H])c6c5[2H])n4)c32)c([2H])c1[2H] ; [2H]c1c([2H])c([2H])c2c(oc3c([2H])c([2H])c(-c4ccc5ccccc5c4)c(-c4nc(-c5ccc(-c6c([2H])c([2H])c([2H])c7c([2H])c([2H])c([2H])c([2H])c67)cc5)nc(-c5ccc6ccccc6c5)n4)c32)c1[2H]]

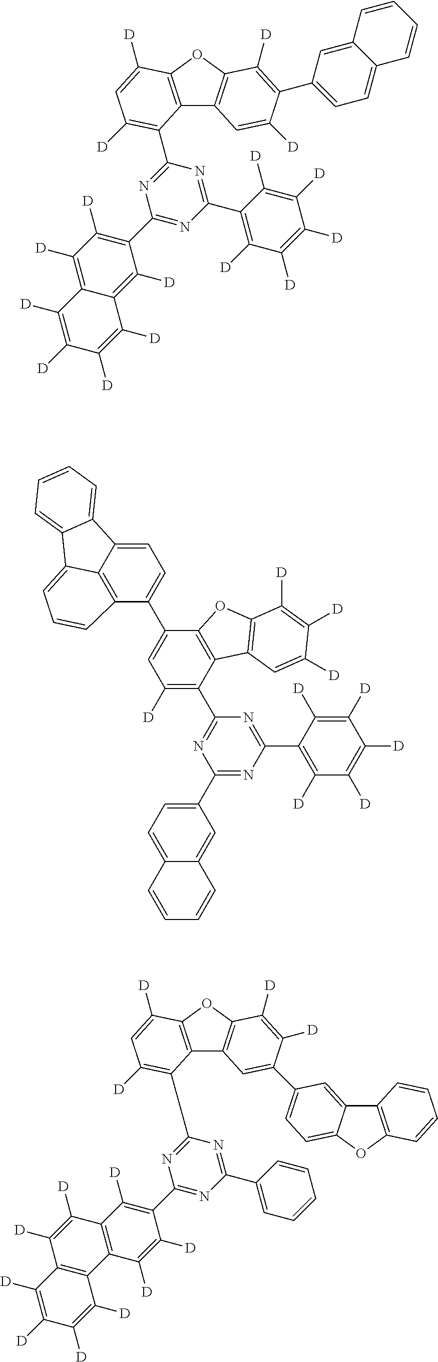 [SMILES: [2H]c1cc2c(oc3c([2H])cc([2H])c(-c4nc(-c5c([2H])c([2H])c([2H])c([2H])c5[2H])nc(-c5c([2H])c([2H])c6c([2H])c([2H])c([2H])c([2H])c6c5[2H])n4)c32)c([2H])c1-c1ccc2ccccc2c1 ; [2H]c1cc2c(oc3c(-c4ccc5c6c(cccc46)-c4ccccc4-5)cc([2H])c(-c4nc(-c5ccc6ccccc6c5)nc(-c5c([2H])c([2H])c([2H])c([2H])c5[2H])n4)c32)c([2H])c1[2H] ; [2H]c1cc([2H])c2oc3c([2H])c([2H])c(-c4ccc5oc6ccccc6c5c4)cc3c2c1-c1nc(-c2ccccc2)nc(-c2c([2H])c([2H])c3c(c2[2H])c([2H])c([2H])c2c([2H])c([2H])c([2H])c([2H])c23)n1]

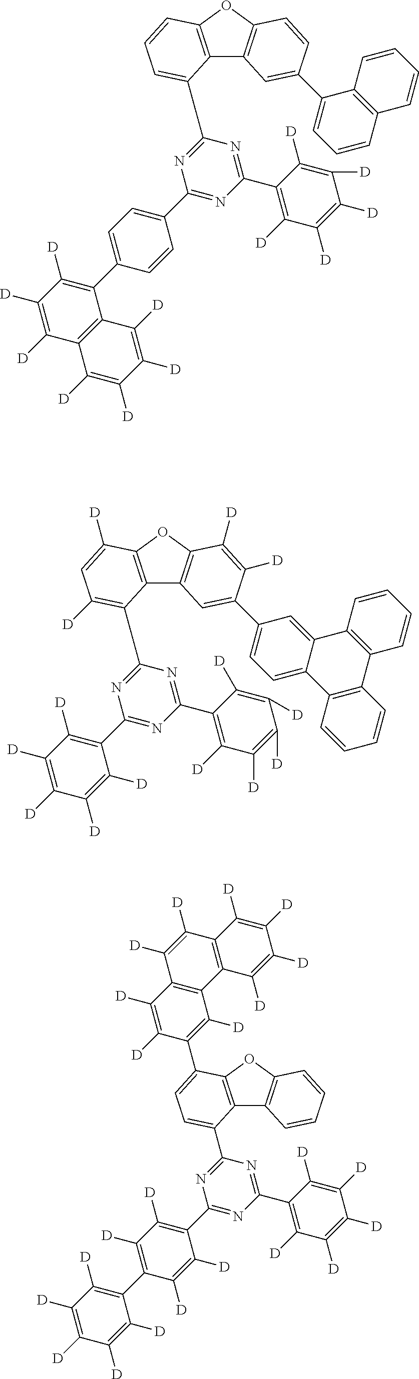

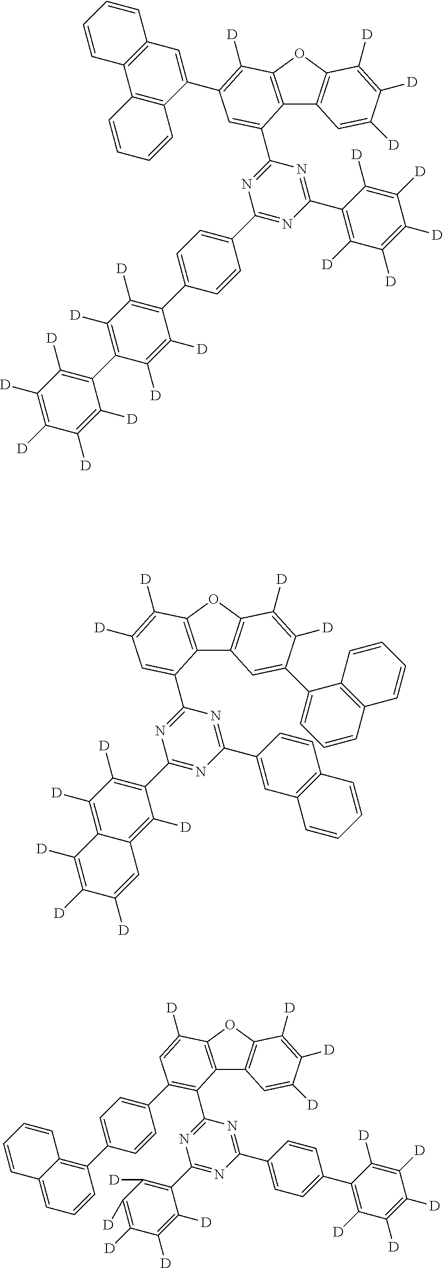

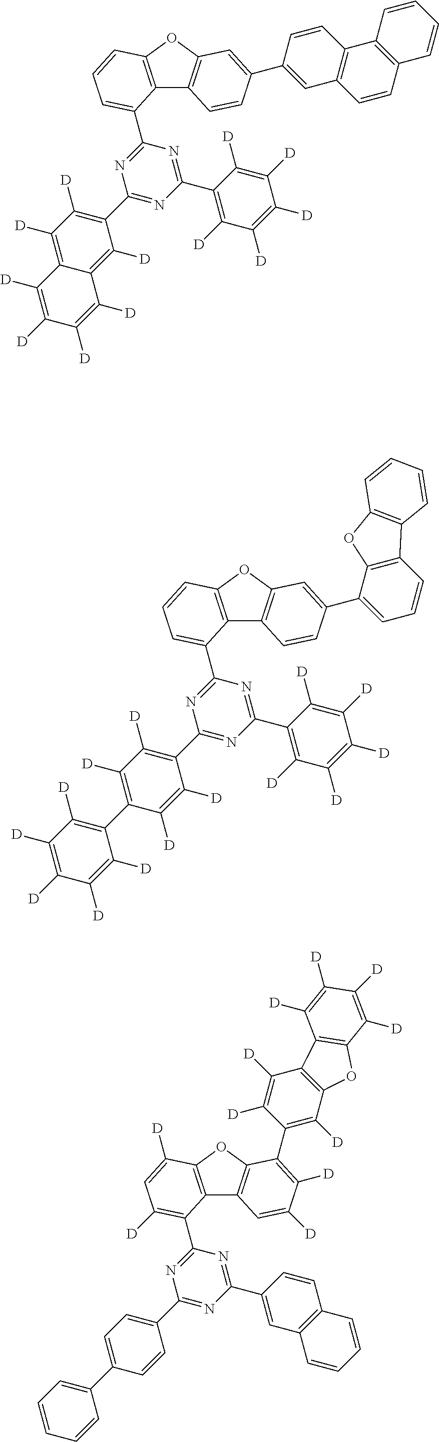 [SMILES: [2H]c1c([2H])c([2H])c(-c2nc(-c3c([2H])c([2H])c4c([2H])c([2H])c([2H])c([2H])c4c3[2H])nc(-c3cccc4oc5cc(-c6ccc7c(ccc8ccccc87)c6)ccc5c34)n2)c([2H])c1[2H] ; [2H]c1c([2H])c([2H])c(-c2nc(-c3c([2H])c([2H])c(-c4c([2H])c([2H])c([2H])c([2H])c4[2H])c([2H])c3[2H])nc(-c3cccc4oc5cc(-c6cccc7c6oc6ccccc67)ccc5c34)n2)c([2H])c1[2H] ; [2H]c1cc2c(oc3c([2H])cc([2H])c(-c4nc(-c5ccc(-c6ccccc6)cc5)nc(-c5ccc6ccccc6c5)n4)c32)c(-c2c([2H])c([2H])c3c(oc4c([2H])c([2H])c([2H])c([2H])c43)c2[2H])c1[2H]]

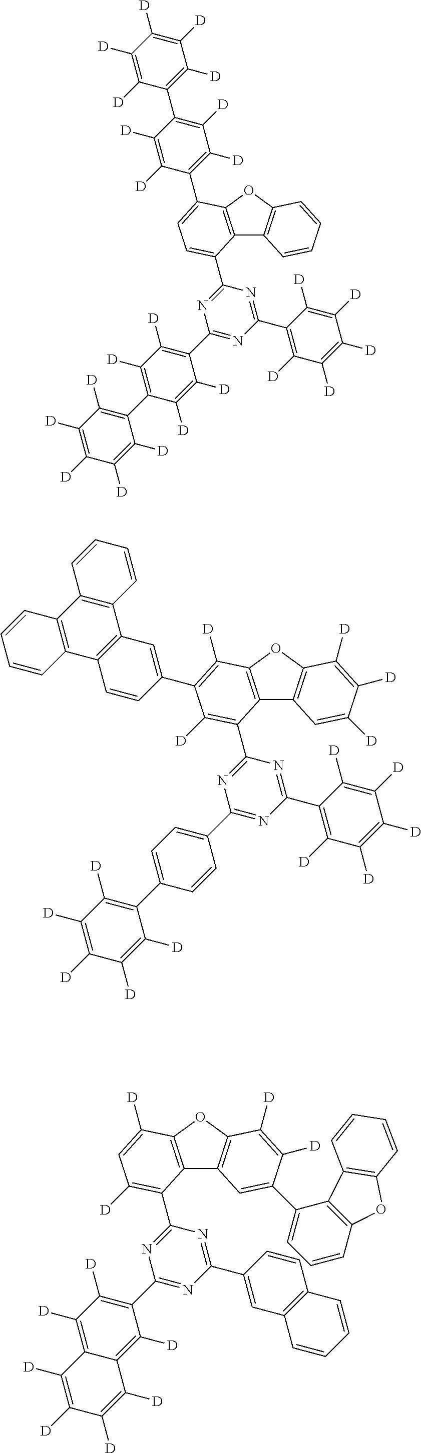

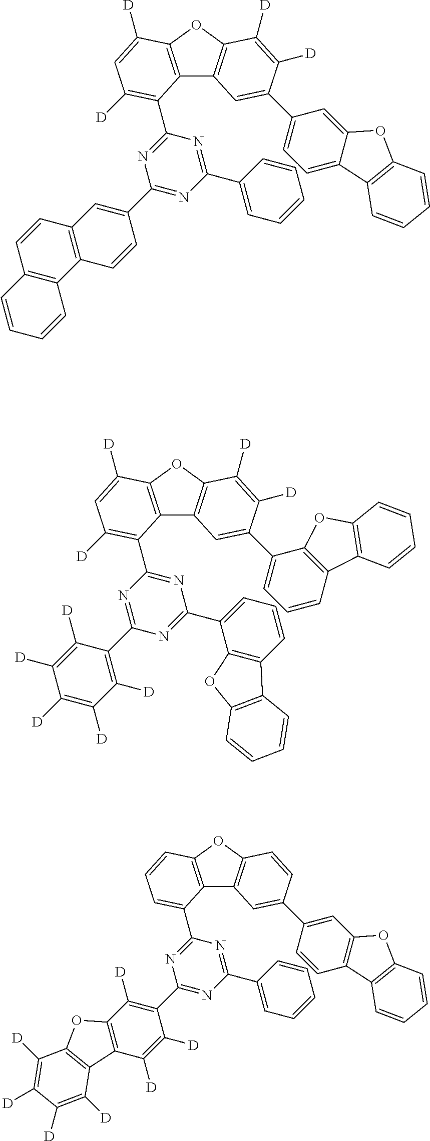 [SMILES: [2H]c1cc([2H])c2oc3c([2H])c([2H])c(-c4ccc5c(c4)oc4ccccc45)cc3c2c1-c1nc(-c2ccccc2)nc(-c2ccc3c(ccc4ccccc43)c2)n1 ; [2H]c1cc([2H])c2oc3c([2H])c([2H])c(-c4cccc5c4oc4ccccc45)cc3c2c1-c1nc(-c2c([2H])c([2H])c([2H])c([2H])c2[2H])nc(-c2cccc3c2oc2ccccc23)n1 ; [2H]c1c([2H])c([2H])c2c(oc3c([2H])c(-c4nc(-c5ccccc5)nc(-c5cccc6oc7ccc(-c8ccc9c(c8)oc8ccccc89)cc7c56)n4)c([2H])c([2H])c32)c1[2H]]

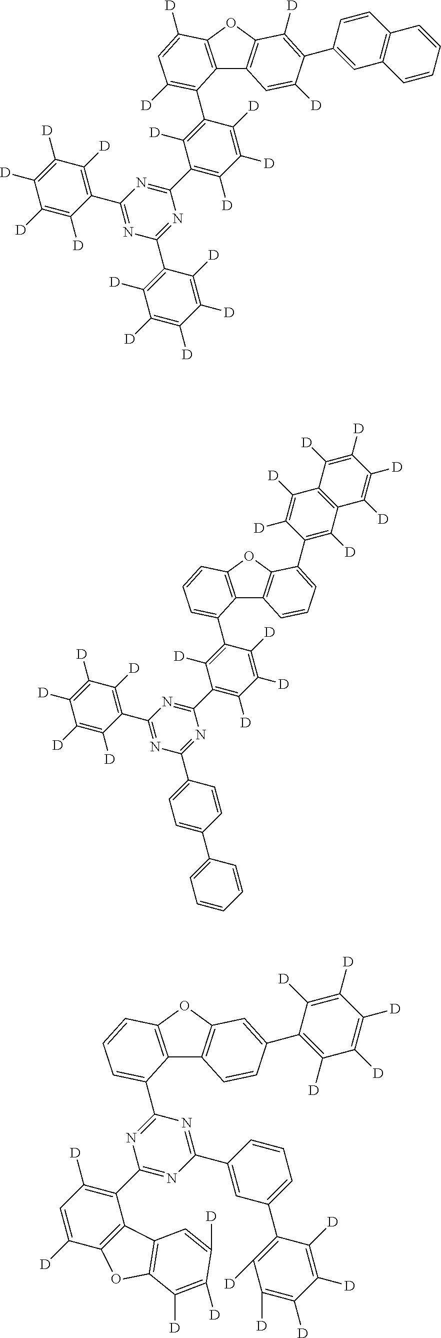

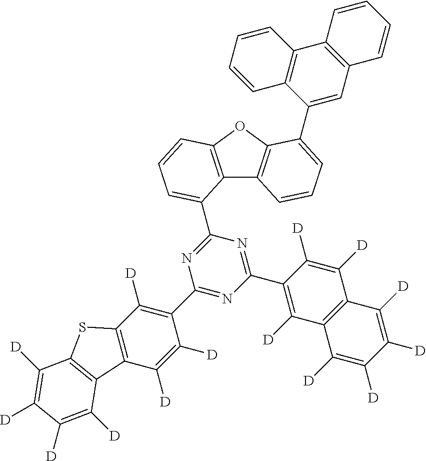 [SMILES: [2H]c1c([2H])c([2H])c2c(sc3c([2H])c(-c4nc(-c5c([2H])c([2H])c6c([2H])c([2H])c([2H])c([2H])c6c5[2H])nc(-c5cccc6oc7c(-c8cc9ccccc9c9ccccc89)cccc7c56)n4)c([2H])c([2H])c32)c1[2H]]

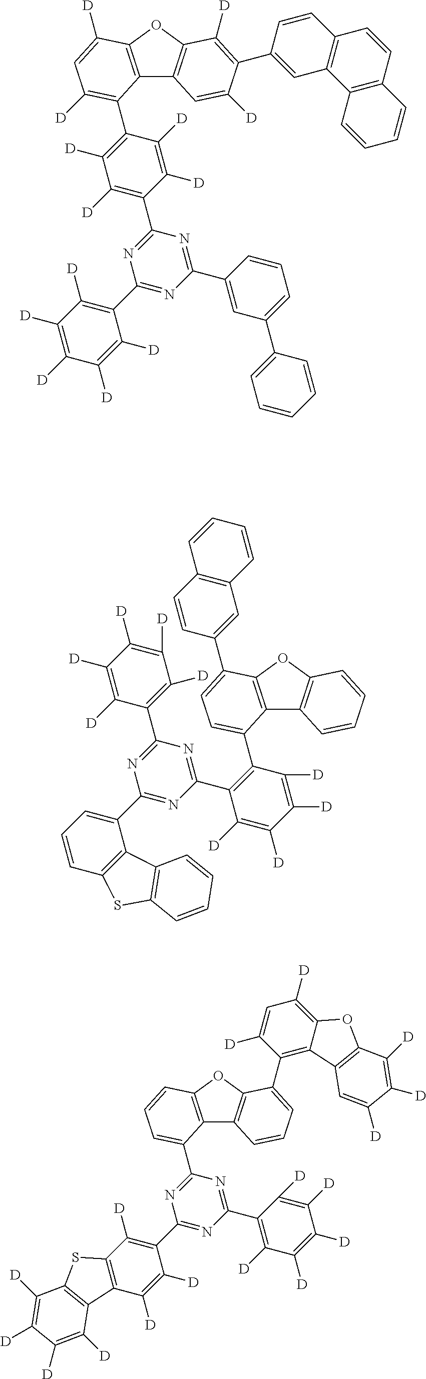 [SMILES: [2H]c1cc2c(oc3c([2H])cc([2H])c(-c4c([2H])c([2H])c(-c5nc(-c6cccc(-c7ccccc7)c6)nc(-c6c([2H])c([2H])c([2H])c([2H])c6[2H])n5)c([2H])c4[2H])c32)c([2H])c1-c1ccc2ccc3ccccc3c2c1 ; [2H]c1c([2H])c([2H])c(-c2nc(-c3c([2H])c([2H])c([2H])c([2H])c3-c3ccc(-c4ccc5ccccc5c4)c4oc5ccccc5c34)nc(-c3cccc4sc5ccccc5c34)n2)c([2H])c1[2H] ; [2H]c1cc2c(oc3c([2H])cc([2H])c(-c4cccc5c4oc4cccc(-c6nc(-c7c([2H])c([2H])c([2H])c([2H])c7[2H])nc(-c7c([2H])c([2H])c8c(sc9c([2H])c([2H])c([2H])c([2H])c98)c7[2H])n6)c45)c32)c([2H])c1[2H]]

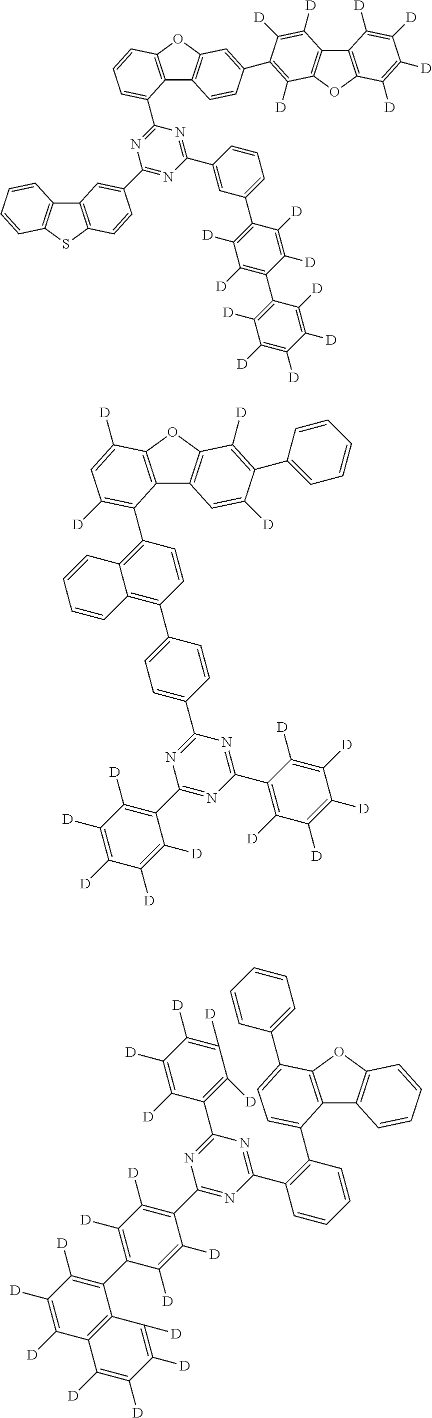

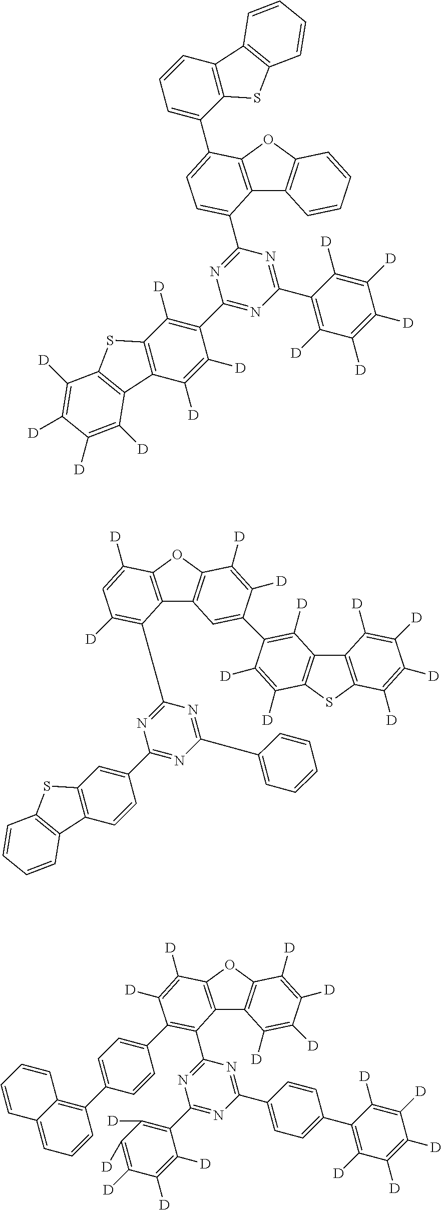 [SMILES: [2H]c1c([2H])c([2H])c(-c2nc(-c3c([2H])c([2H])c4c(sc5c([2H])c([2H])c([2H])c([2H])c54)c3[2H])nc(-c3ccc(-c4cccc5c4sc4ccccc45)c4oc5ccccc5c34)n2)c([2H])c1[2H] ; [2H]c1cc([2H])c2oc3c([2H])c([2H])c(-c4c([2H])c([2H])c5sc6c([2H])c([2H])c([2H])c([2H])c6c5c4[2H])cc3c2c1-c1nc(-c2ccccc2)nc(-c2ccc3c(c2)sc2ccccc23)n1 ; [2H]c1c([2H])c([2H])c(-c2ccc(-c3nc(-c4c([2H])c([2H])c([2H])c([2H])c4[2H])nc(-c4c(-c5ccc(-c6cccc7ccccc67)cc5)c([2H])c([2H])c5oc6c([2H])c([2H])c([2H])c([2H])c6c45)n3)cc2)c([2H])c1[2H]]

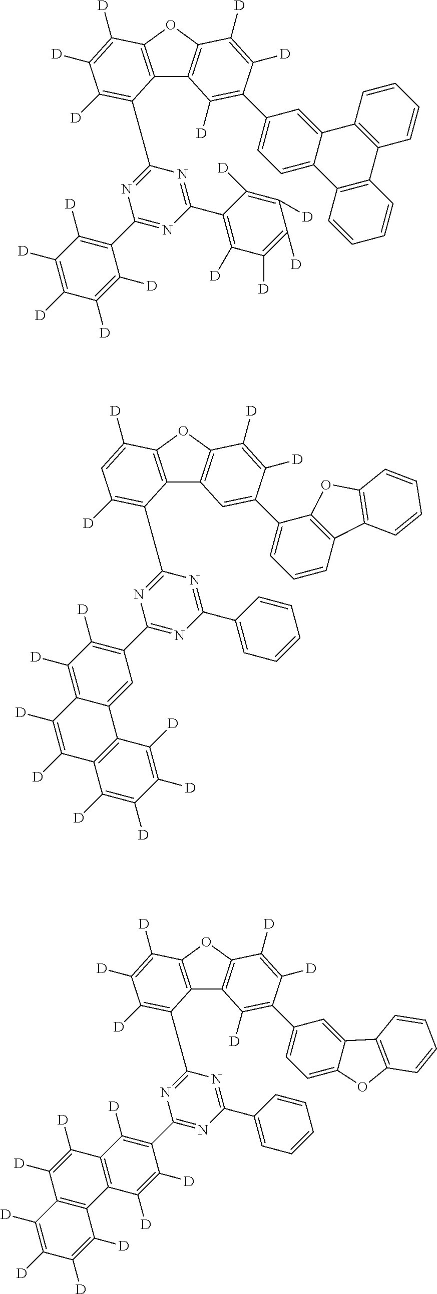

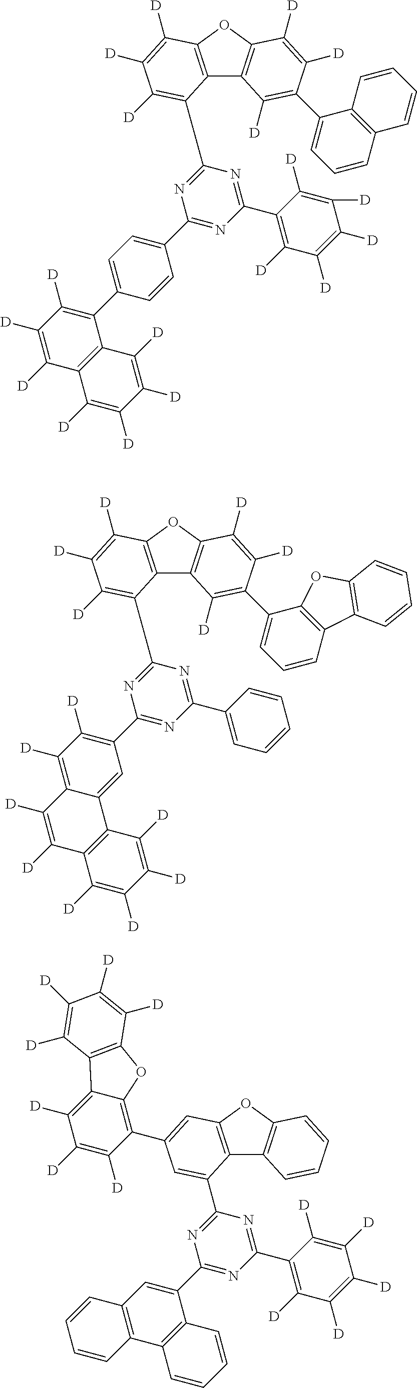

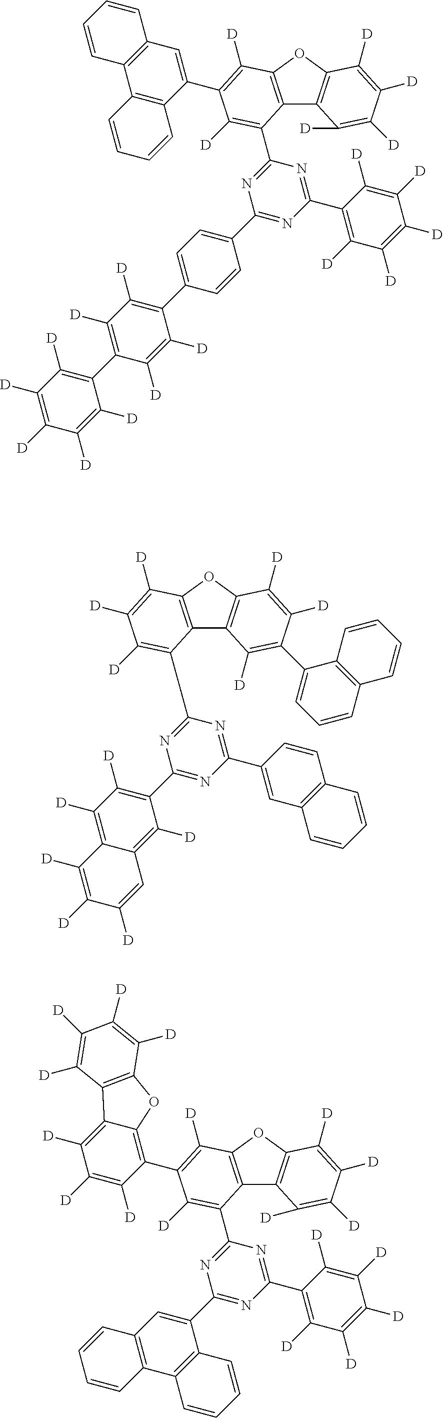

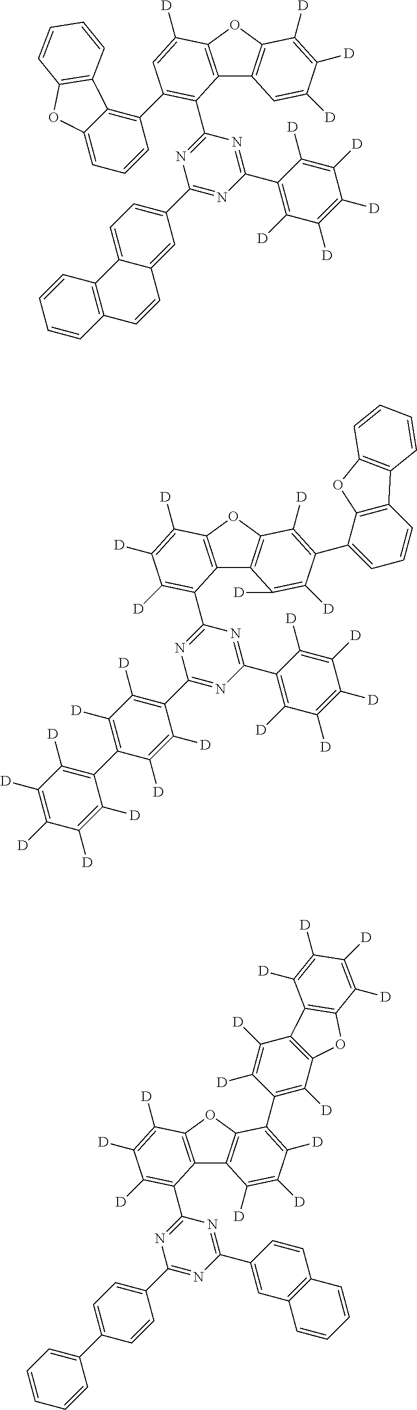

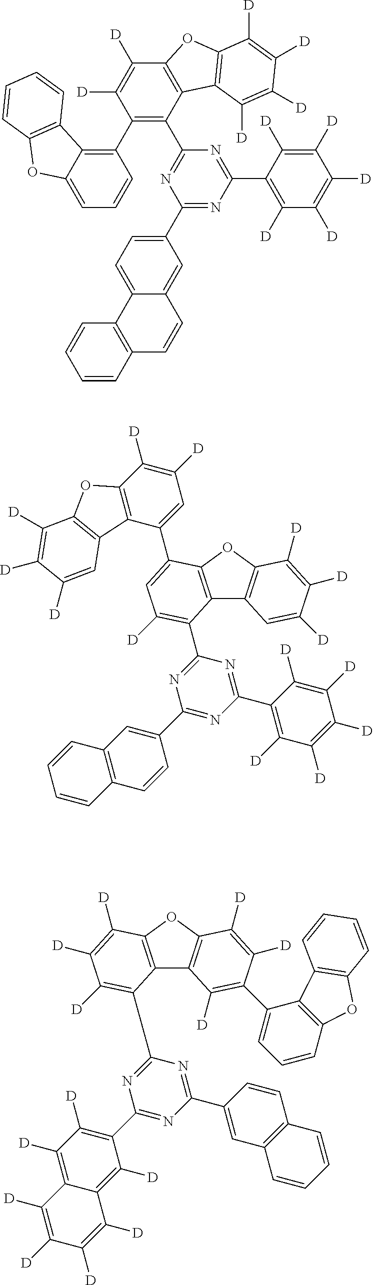 [SMILES: [2H]c1c([2H])c([2H])c(-c2nc(-c3ccc4c(ccc5ccccc54)c3)nc(-c3c(-c4cccc5oc6ccccc6c45)c([2H])c([2H])c4oc5c([2H])c([2H])c([2H])c([2H])c5c34)n2)c([2H])c1[2H] ; [2H]c1cc2c(oc3c([2H])c([2H])cc(-c4cc([2H])c(-c5nc(-c6ccc7ccccc7c6)nc(-c6c([2H])c([2H])c([2H])c([2H])c6[2H])n5)c5c4oc4c([2H])c([2H])c([2H])cc45)c32)c([2H])c1[2H] ; [2H]c1c(-c2cccc3oc4ccccc4c23)c([2H])c2c(oc3c([2H])c([2H])c([2H])c(-c4nc(-c5ccc6ccccc6c5)nc(-c5c([2H])c([2H])c6c([2H])c([2H])c([2H])c([2H])c6c5[2H])n4)c32)c1[2H]]

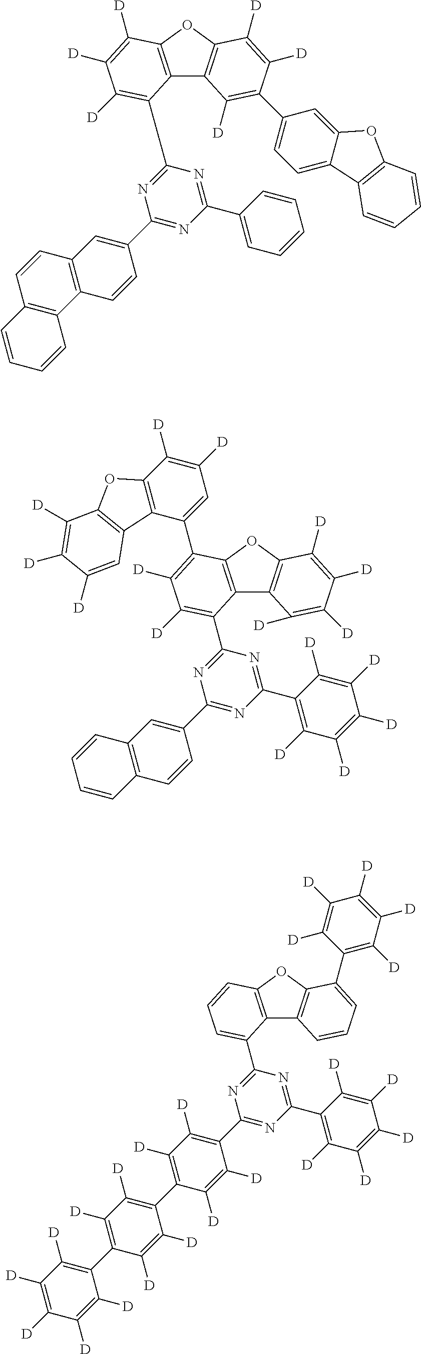

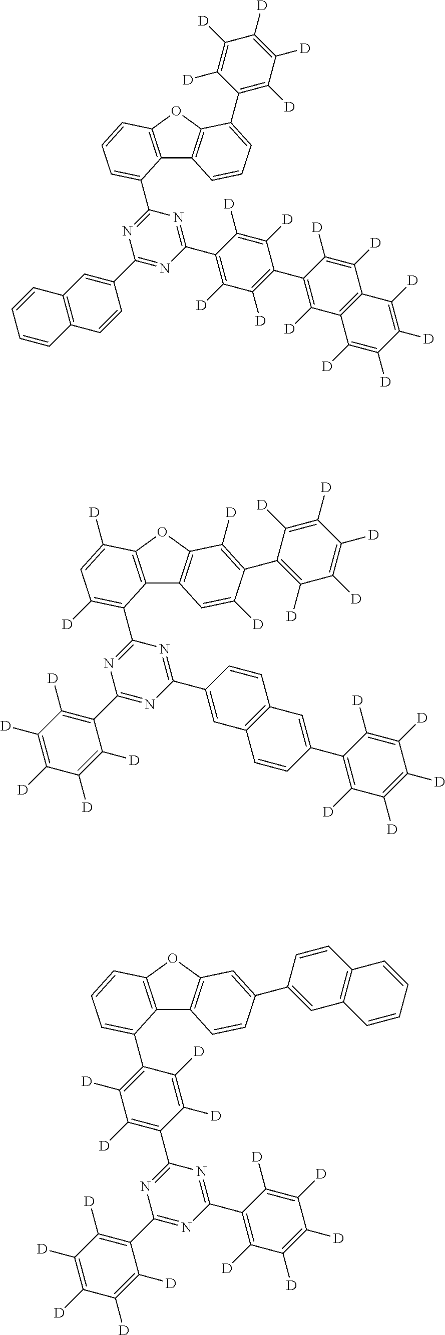

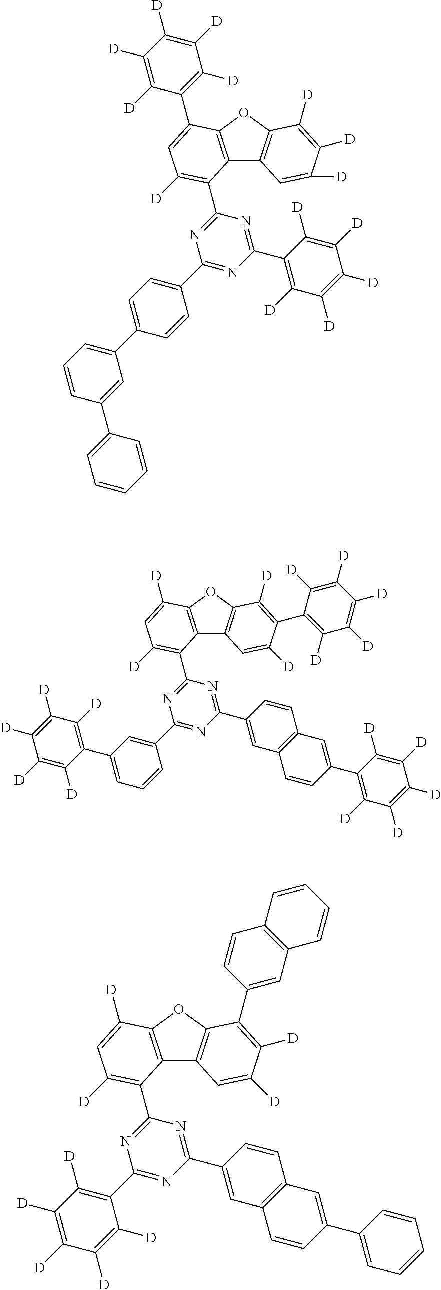

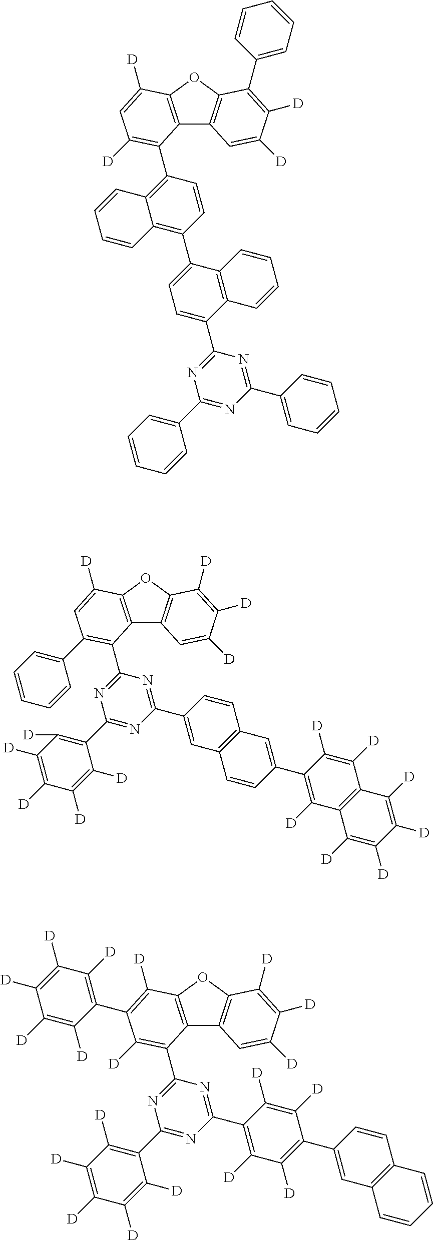 [SMILES: [2H]c1cc2c(oc3c([2H])cc([2H])c(-c4ccc(-c5ccc(-c6nc(-c7ccccc7)nc(-c7ccccc7)n6)c6ccccc56)c5ccccc45)c32)c(-c2ccccc2)c1[2H] ; [2H]c1cc2c(oc3c([2H])cc(-c4ccccc4)c(-c4nc(-c5ccc6cc(-c7c([2H])c([2H])c8c([2H])c([2H])c([2H])c([2H])c8c7[2H])ccc6c5)nc(-c5c([2H])c([2H])c([2H])c([2H])c5[2H])n4)c32)c([2H])c1[2H] ; [2H]c1cc2c(oc3c([2H])c(-c4c([2H])c([2H])c([2H])c([2H])c4[2H])c([2H])c(-c4nc(-c5c([2H])c([2H])c([2H])c([2H])c5[2H])nc(-c5c([2H])c([2H])c(-c6ccc7ccccc7c6)c([2H])c5[2H])n4)c32)c([2H])c1[2H]]

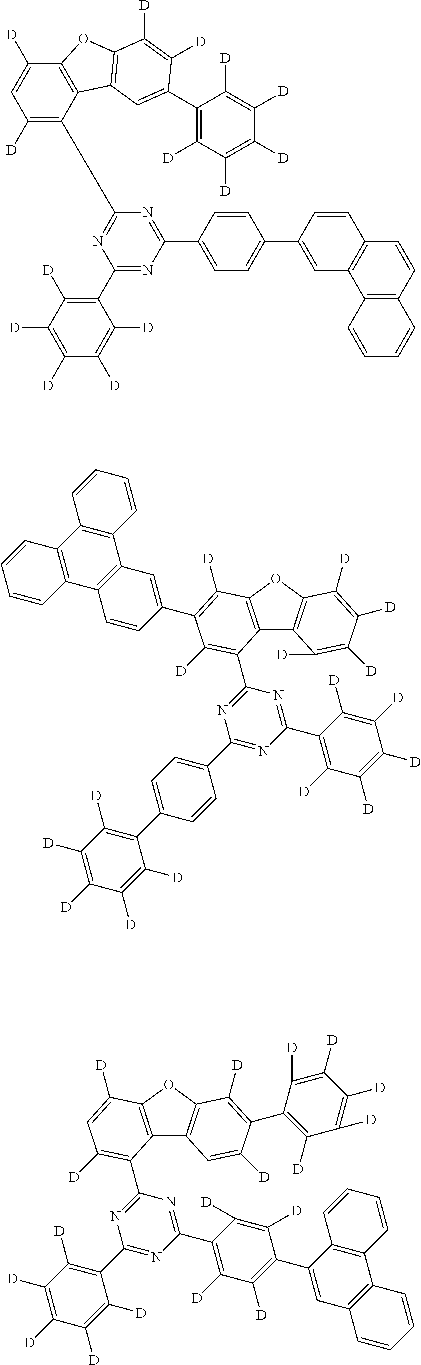

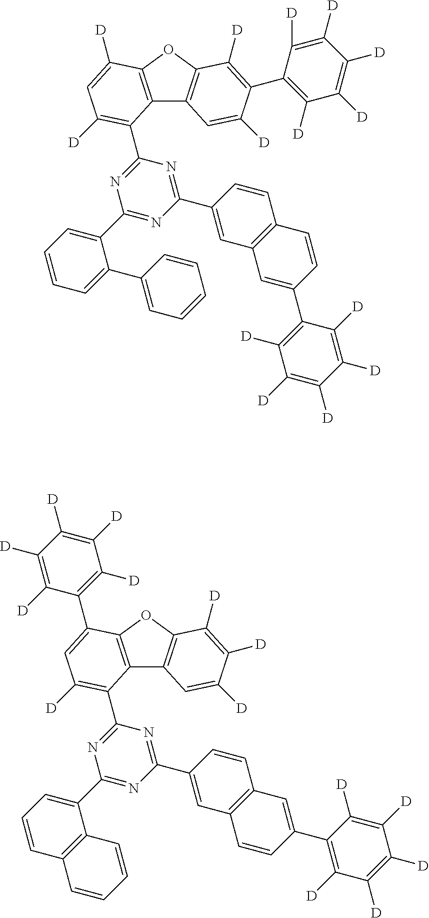 [SMILES: [2H]c1cc2c(oc3c([2H])cc([2H])c(-c4nc(-c5ccc6ccc(-c7c([2H])c([2H])c([2H])c([2H])c7[2H])cc6c5)nc(-c5ccccc5-c5ccccc5)n4)c32)c([2H])c1-c1c([2H])c([2H])c([2H])c([2H])c1[2H] ; [2H]c1cc2c(oc3c(-c4c([2H])c([2H])c([2H])c([2H])c4[2H])cc([2H])c(-c4nc(-c5ccc6cc(-c7c([2H])c([2H])c([2H])c([2H])c7[2H])ccc6c5)nc(-c5cccc6ccccc56)n4)c32)c([2H])c1[2H]]

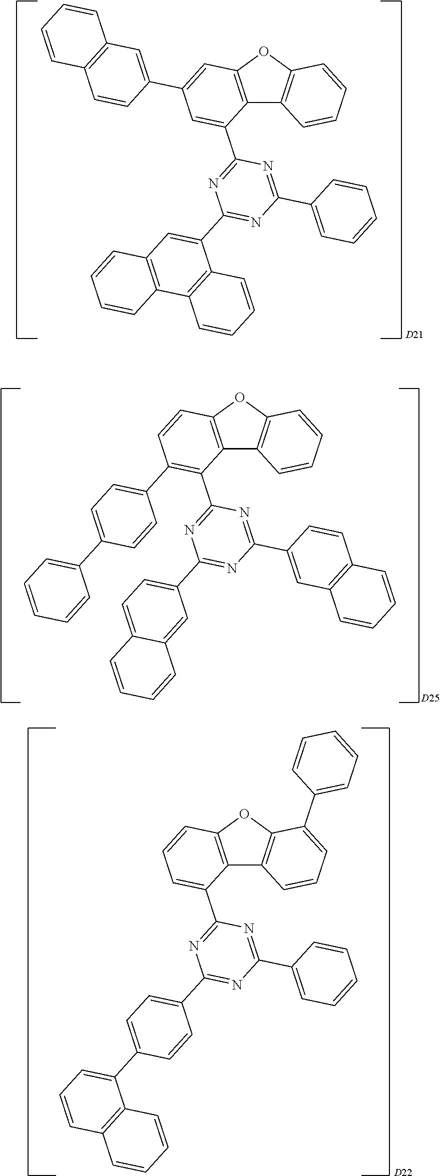

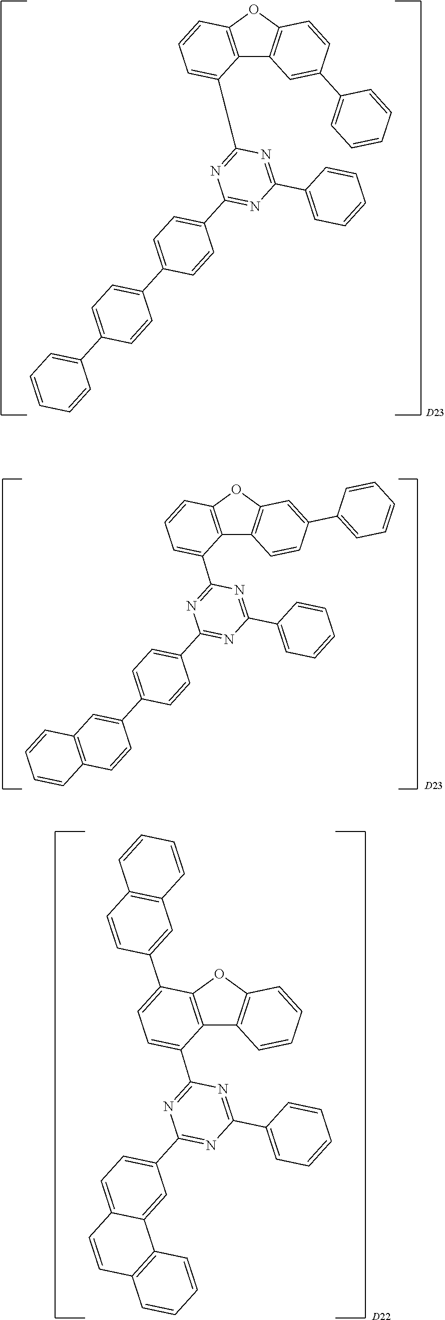

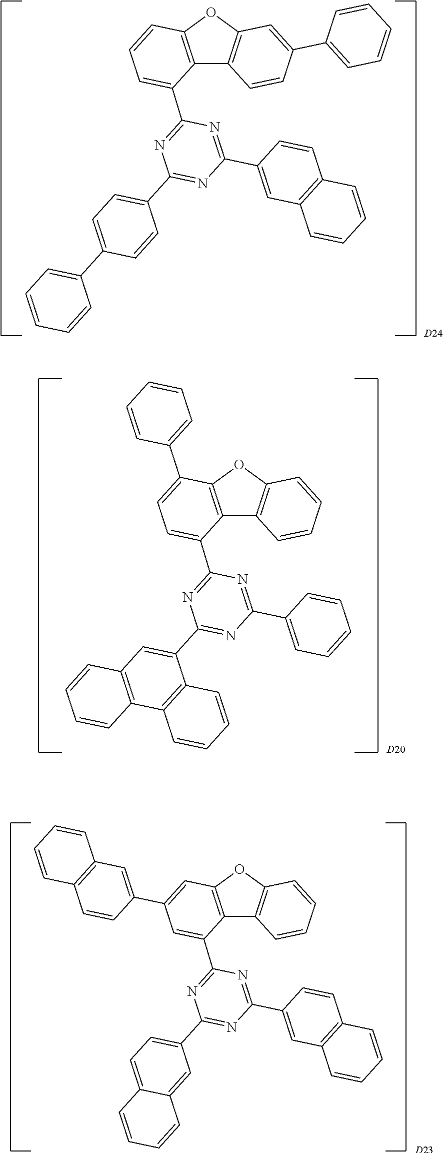

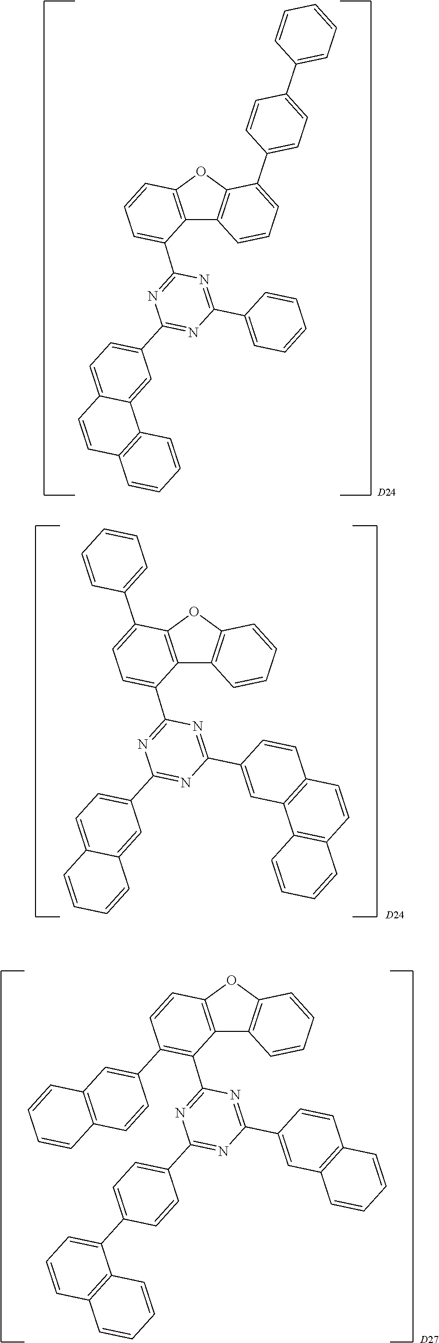

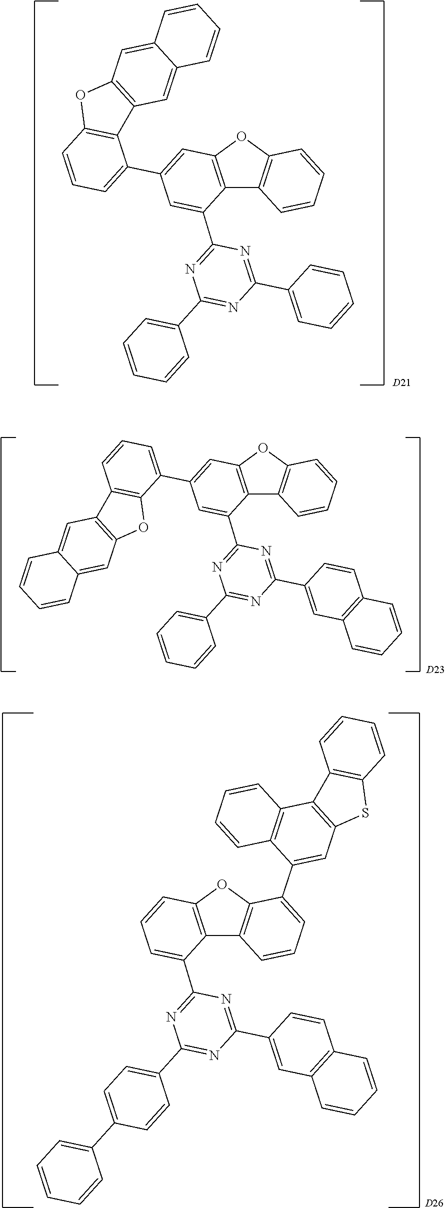

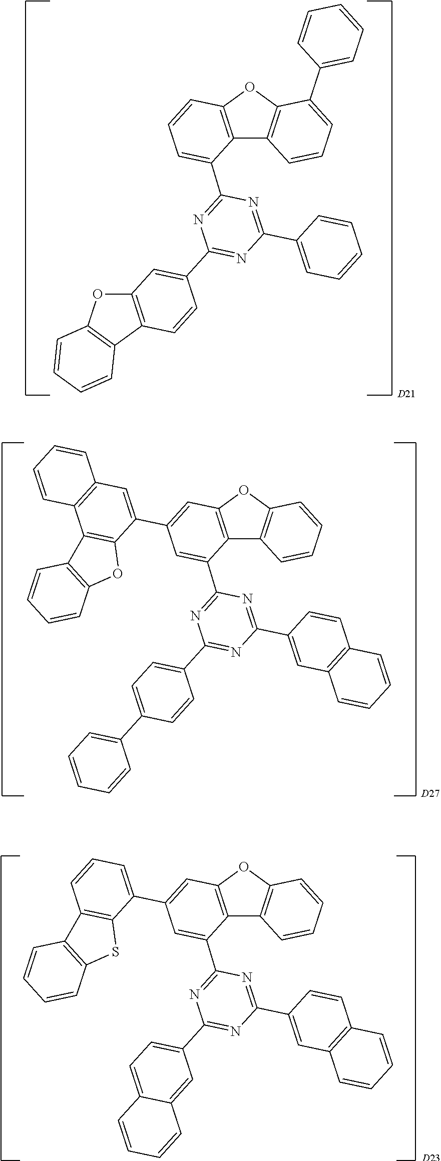

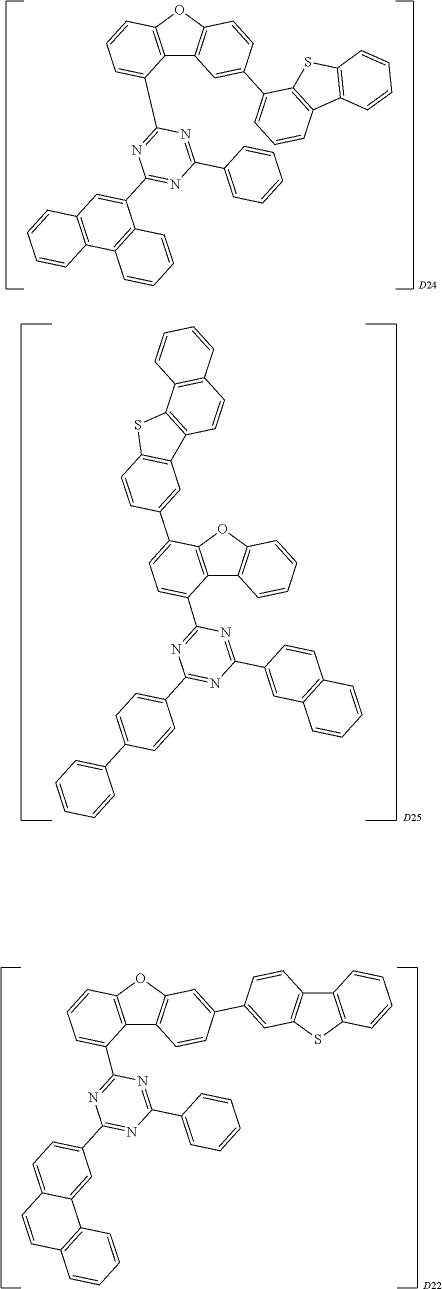

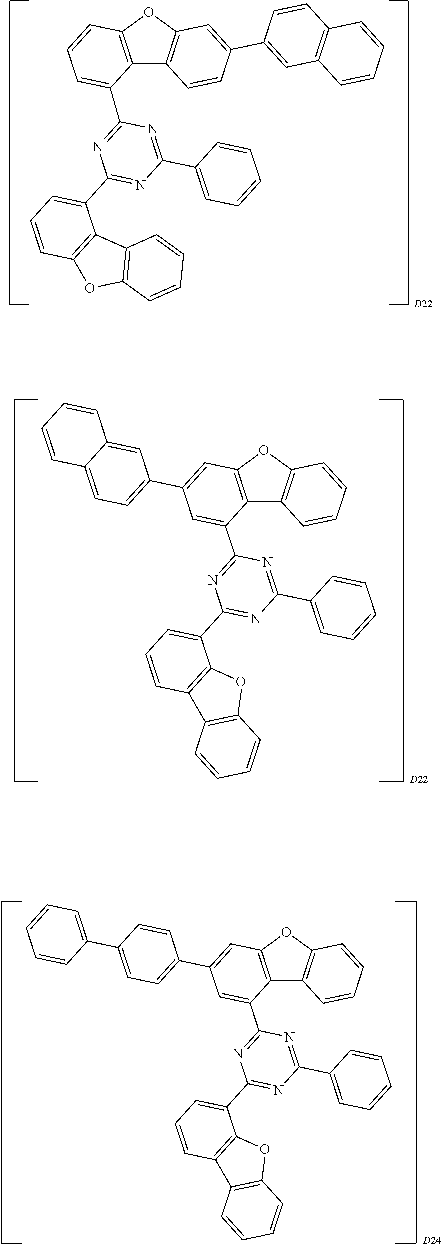

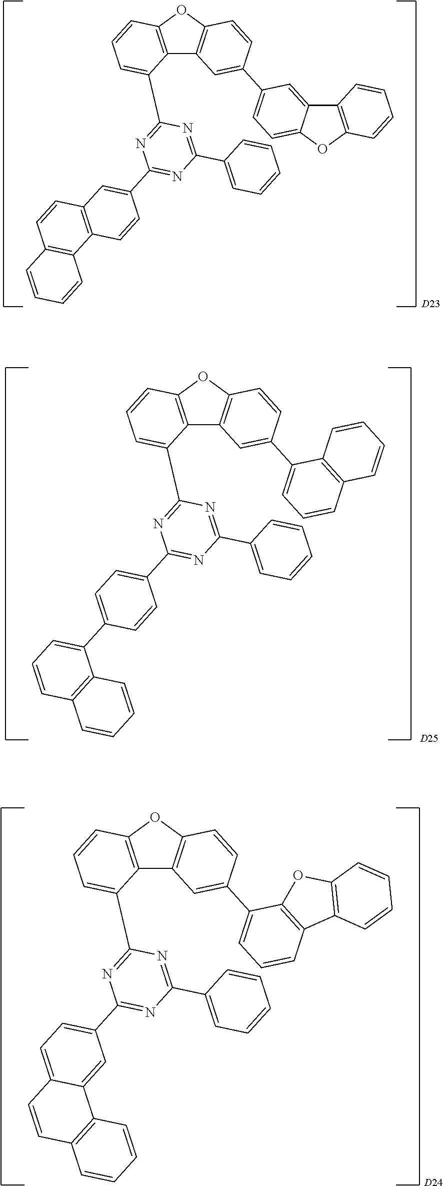

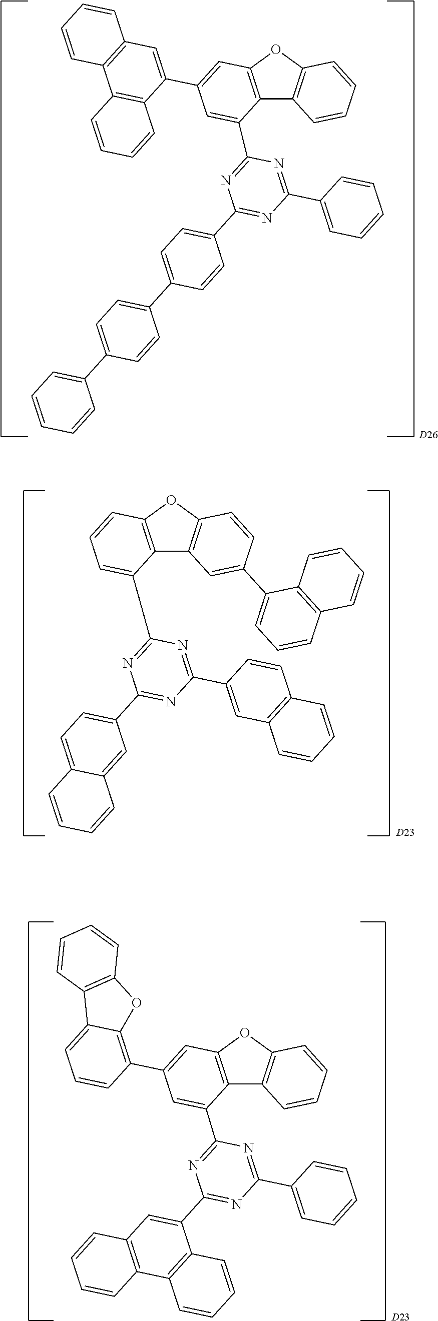

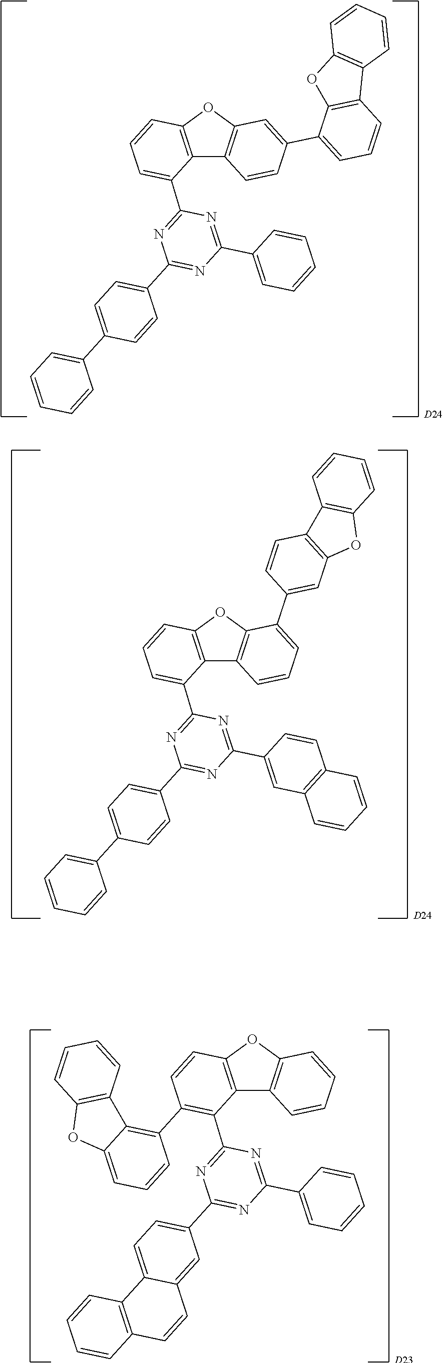

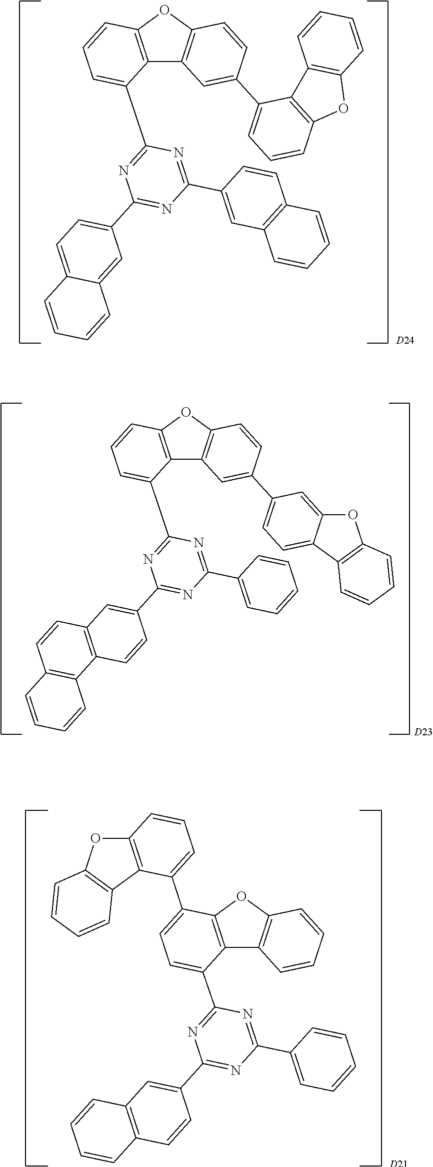

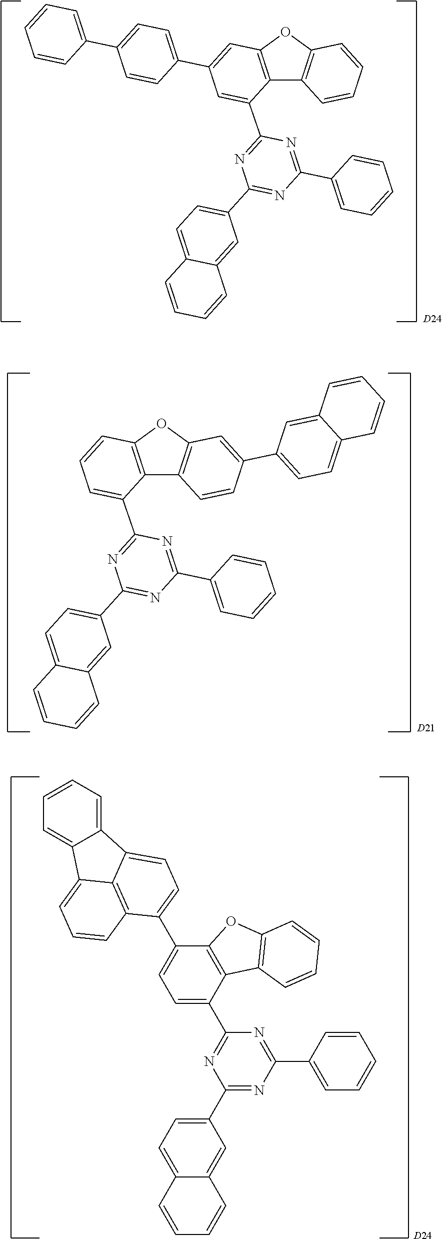

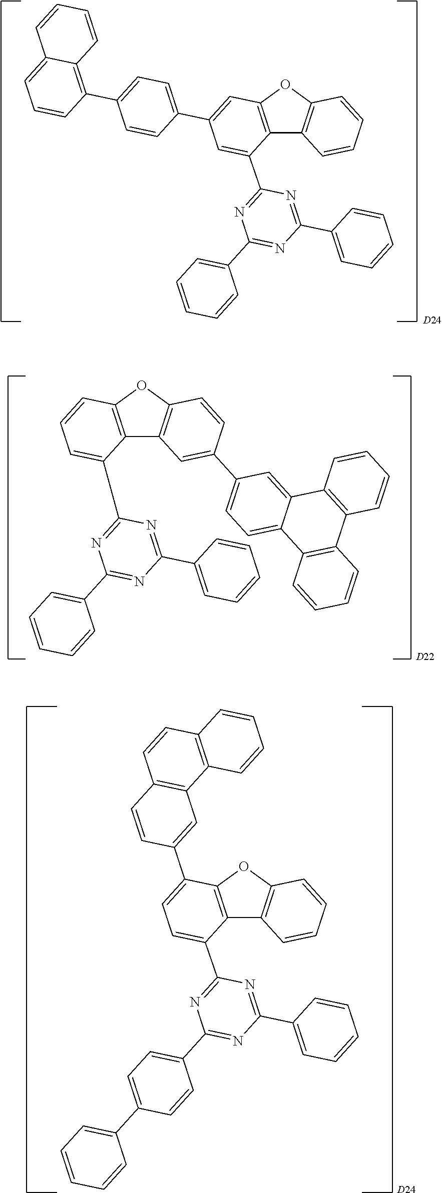

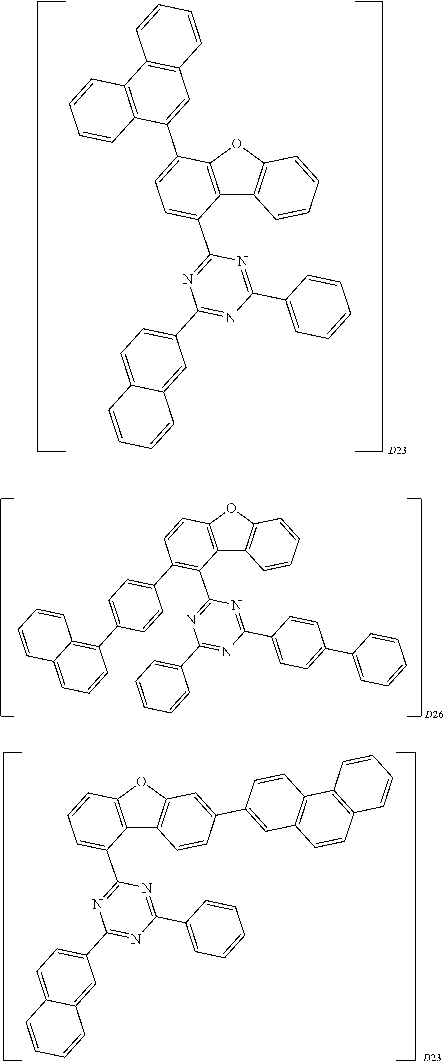

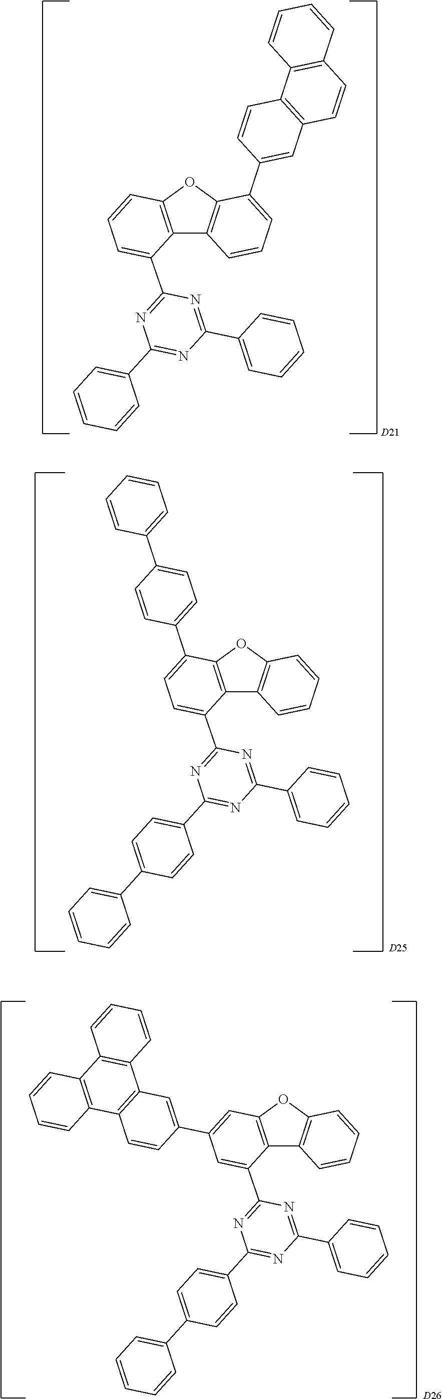

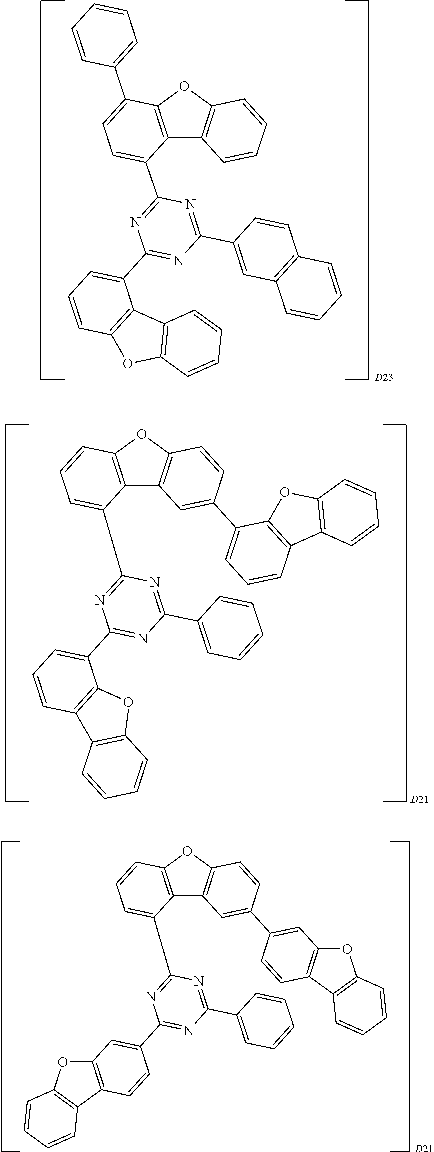

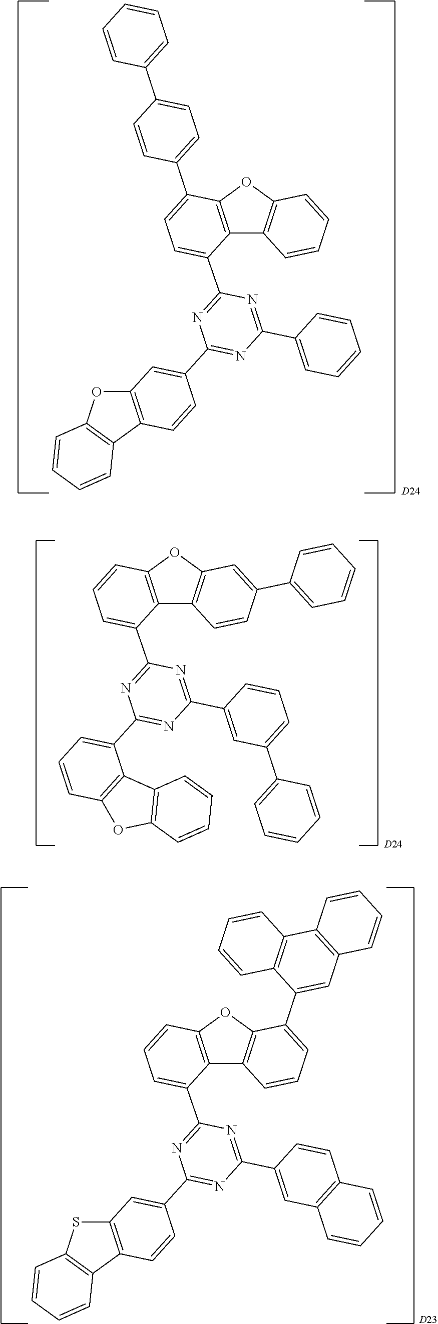

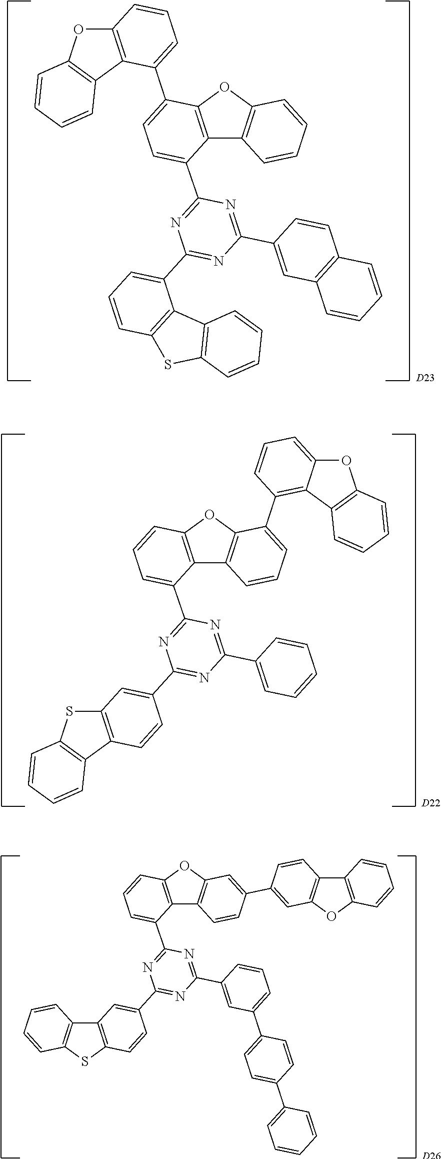

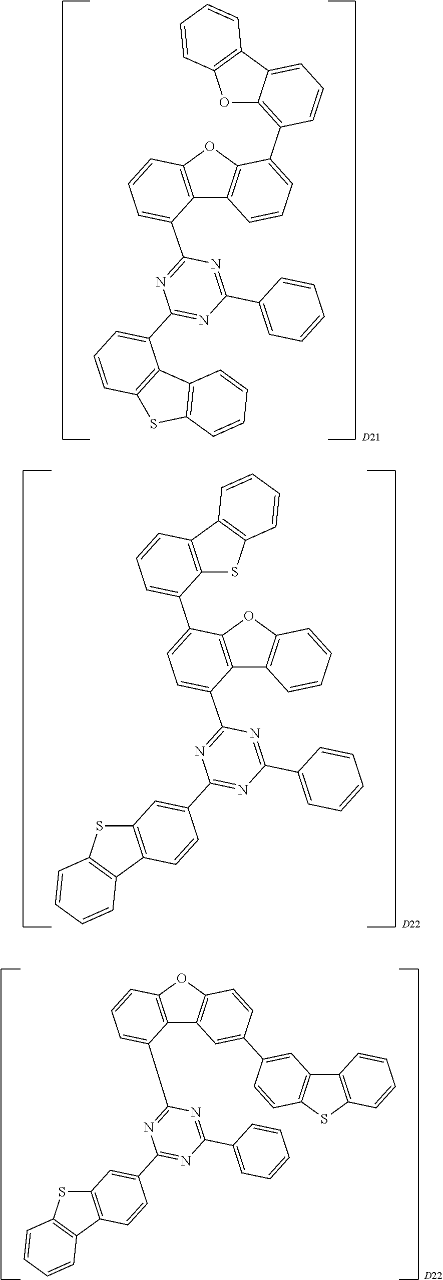

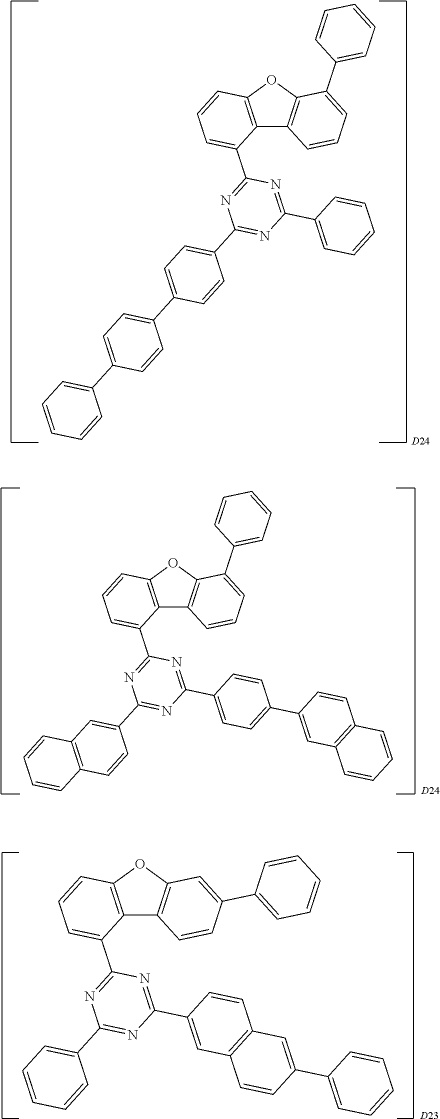

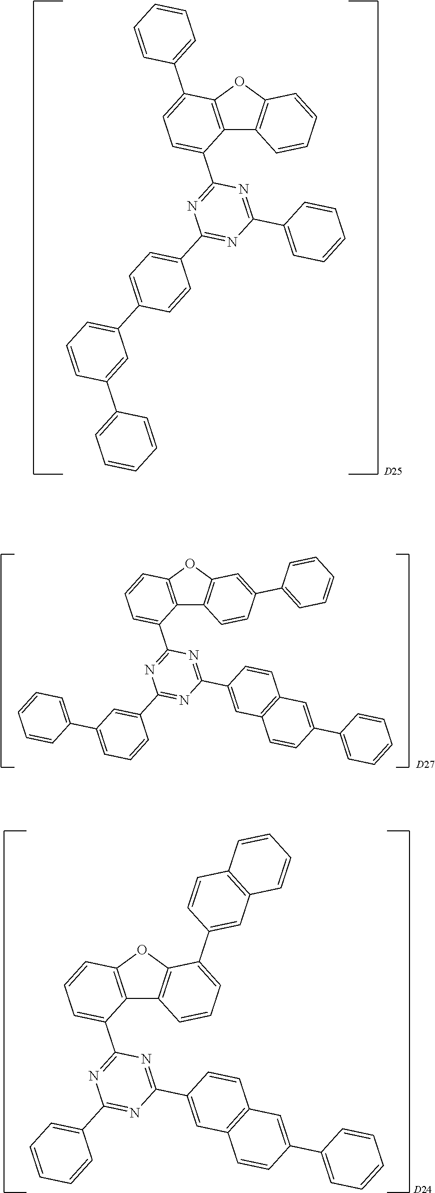

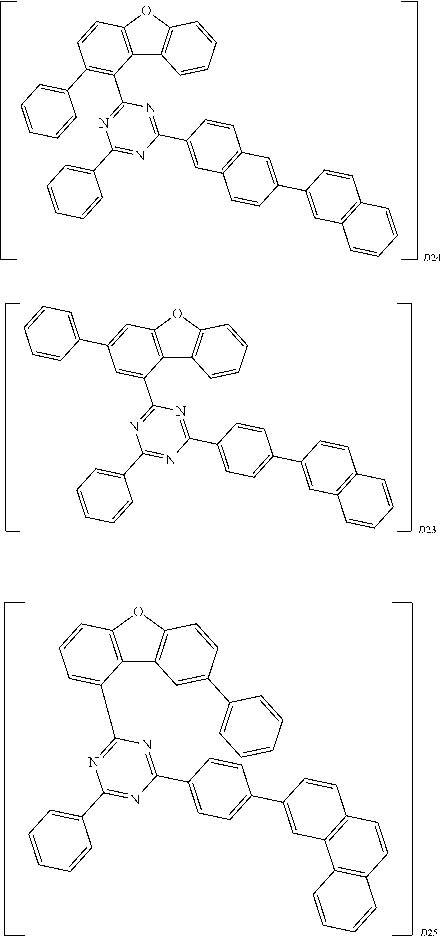

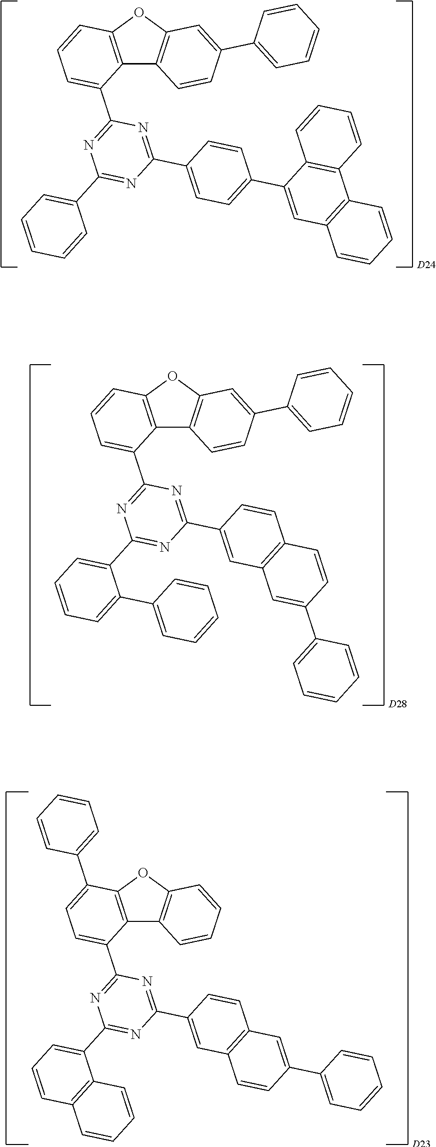

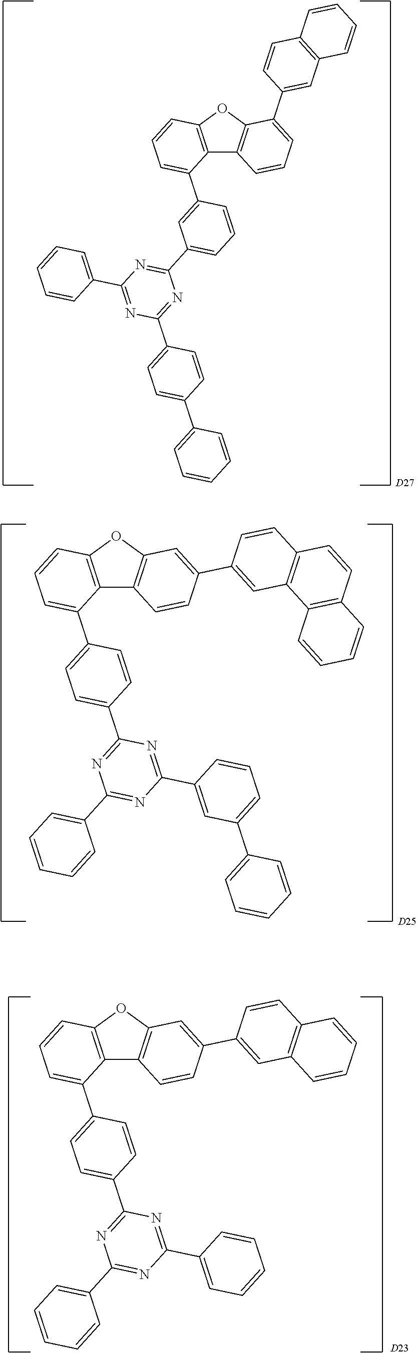

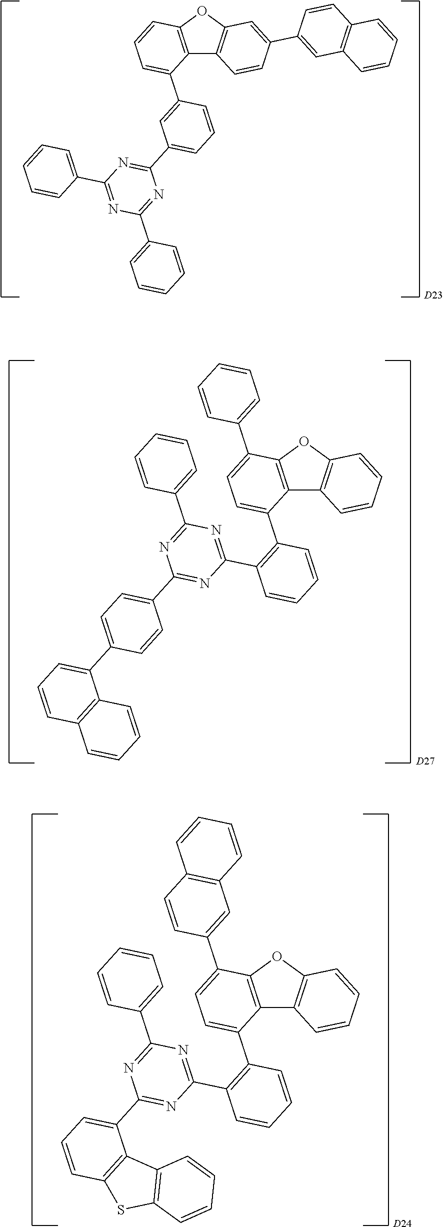

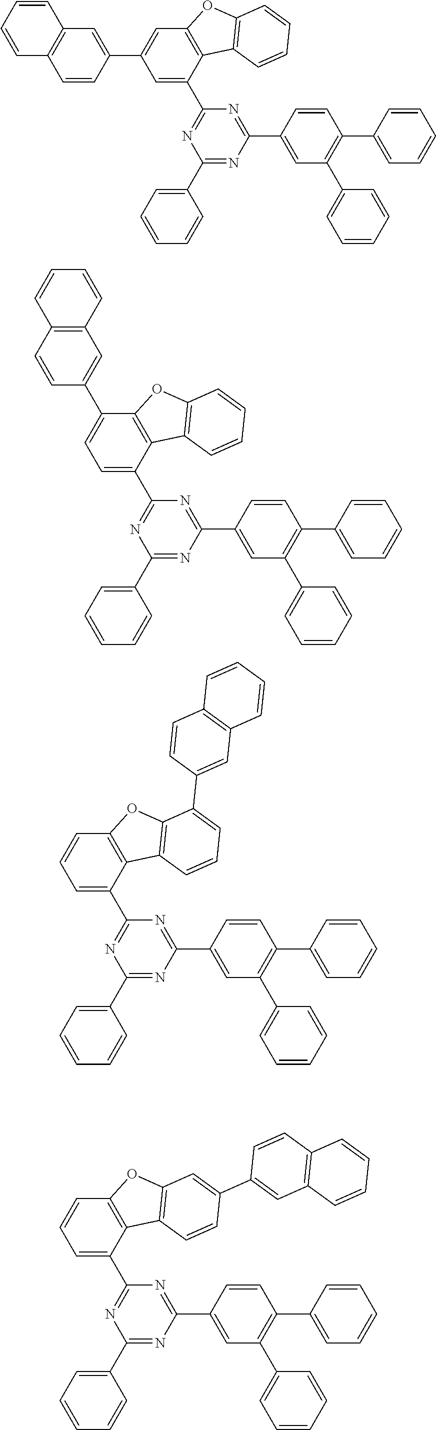 [SMILES: c1ccc(-c2nc(-c3ccc(-c4ccccc4)c(-c4ccccc4)c3)nc(-c3cc(-c4ccc5ccccc5c4)cc4oc5ccccc5c34)n2)cc1 ; c1ccc(-c2nc(-c3ccc(-c4ccccc4)c(-c4ccccc4)c3)nc(-c3ccc(-c4ccc5ccccc5c4)c4oc5ccccc5c34)n2)cc1 ; c1ccc(-c2nc(-c3ccc(-c4ccccc4)c(-c4ccccc4)c3)nc(-c3cccc4oc5c(-c6ccc7ccccc7c6)cccc5c34)n2)cc1 ; c1ccc(-c2nc(-c3ccc(-c4ccccc4)c(-c4ccccc4)c3)nc(-c3cccc4oc5cc(-c6ccc7ccccc7c6)ccc5c34)n2)cc1]

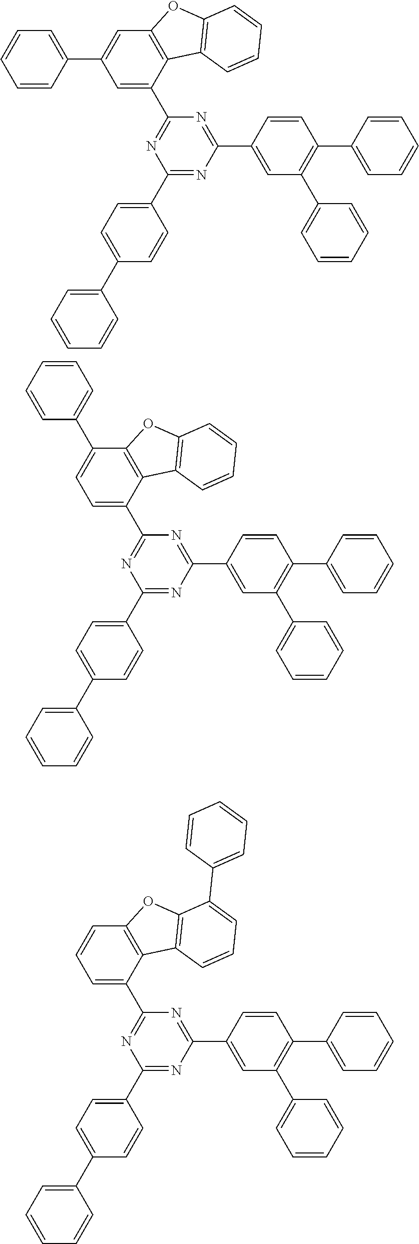

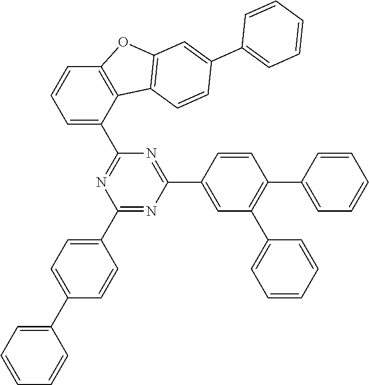 [SMILES: c1ccc(-c2ccc(-c3nc(-c4ccc(-c5ccccc5)c(-c5ccccc5)c4)nc(-c4cccc5oc6cc(-c7ccccc7)ccc6c45)n3)cc2)cc1]

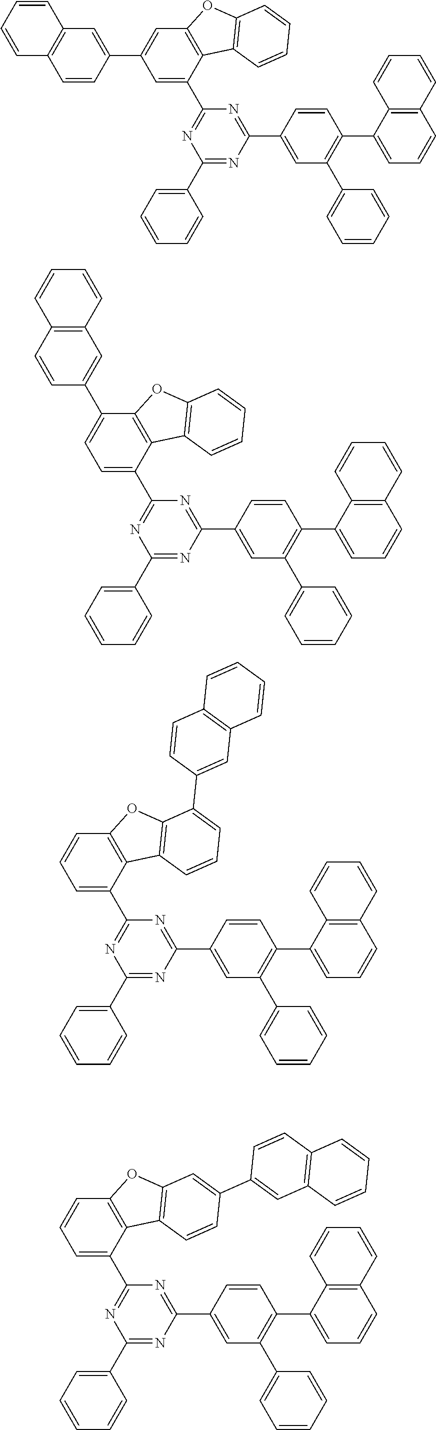 [SMILES: c1ccc(-c2nc(-c3ccc(-c4cccc5ccccc45)c(-c4ccccc4)c3)nc(-c3cc(-c4ccc5ccccc5c4)cc4oc5ccccc5c34)n2)cc1 ; c1ccc(-c2nc(-c3ccc(-c4cccc5ccccc45)c(-c4ccccc4)c3)nc(-c3ccc(-c4ccc5ccccc5c4)c4oc5ccccc5c34)n2)cc1 ; c1ccc(-c2nc(-c3ccc(-c4cccc5ccccc45)c(-c4ccccc4)c3)nc(-c3cccc4oc5c(-c6ccc7ccccc7c6)cccc5c34)n2)cc1 ; c1ccc(-c2nc(-c3ccc(-c4cccc5ccccc45)c(-c4ccccc4)c3)nc(-c3cccc4oc5cc(-c6ccc7ccccc7c6)ccc5c34)n2)cc1]

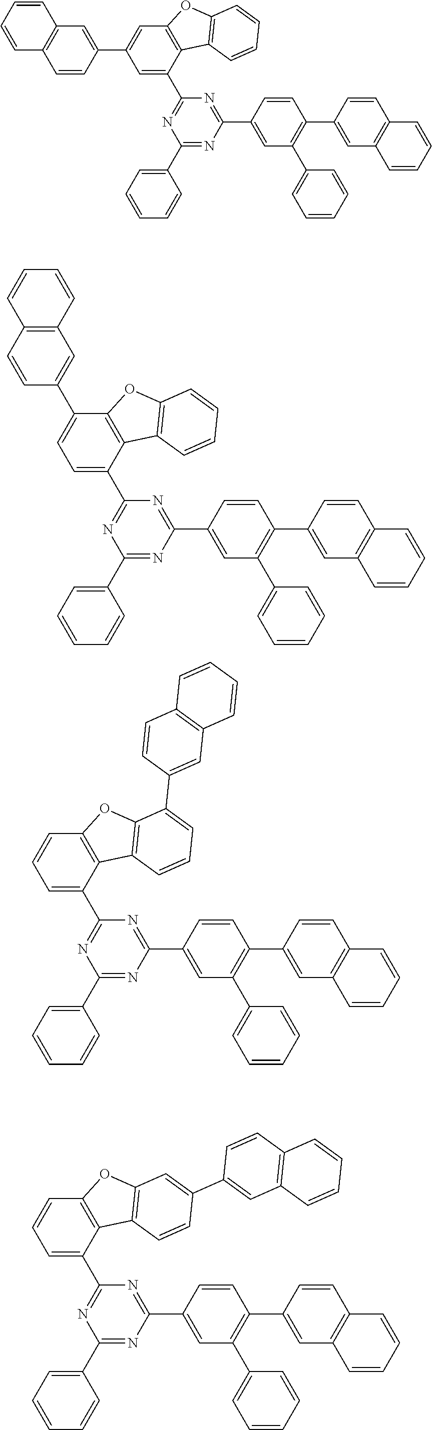 [SMILES: c1ccc(-c2nc(-c3ccc(-c4ccc5ccccc5c4)c(-c4ccccc4)c3)nc(-c3cc(-c4ccc5ccccc5c4)cc4oc5ccccc5c34)n2)cc1 ; c1ccc(-c2nc(-c3ccc(-c4ccc5ccccc5c4)c(-c4ccccc4)c3)nc(-c3ccc(-c4ccc5ccccc5c4)c4oc5ccccc5c34)n2)cc1 ; c1ccc(-c2nc(-c3ccc(-c4ccc5ccccc5c4)c(-c4ccccc4)c3)nc(-c3cccc4oc5c(-c6ccc7ccccc7c6)cccc5c34)n2)cc1 ; c1ccc(-c2nc(-c3ccc(-c4ccc5ccccc5c4)c(-c4ccccc4)c3)nc(-c3cccc4oc5cc(-c6ccc7ccccc7c6)ccc5c34)n2)cc1]

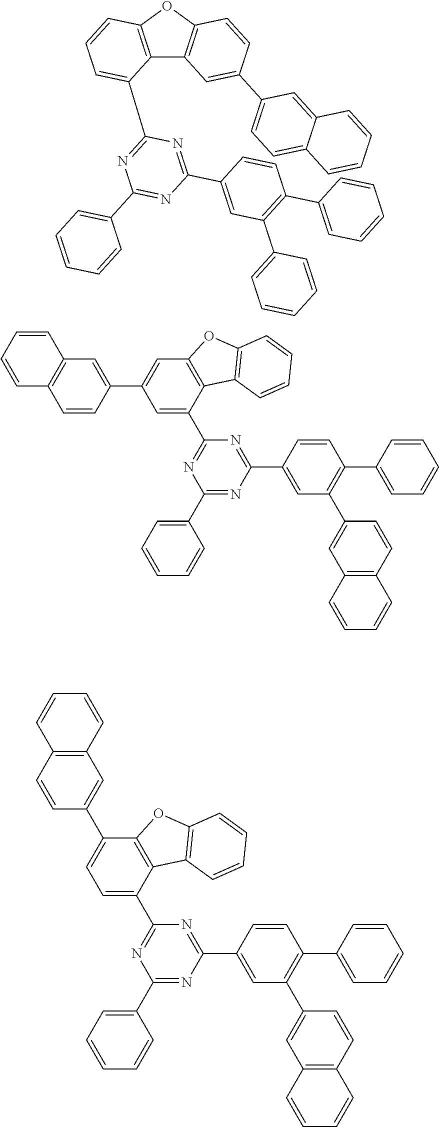

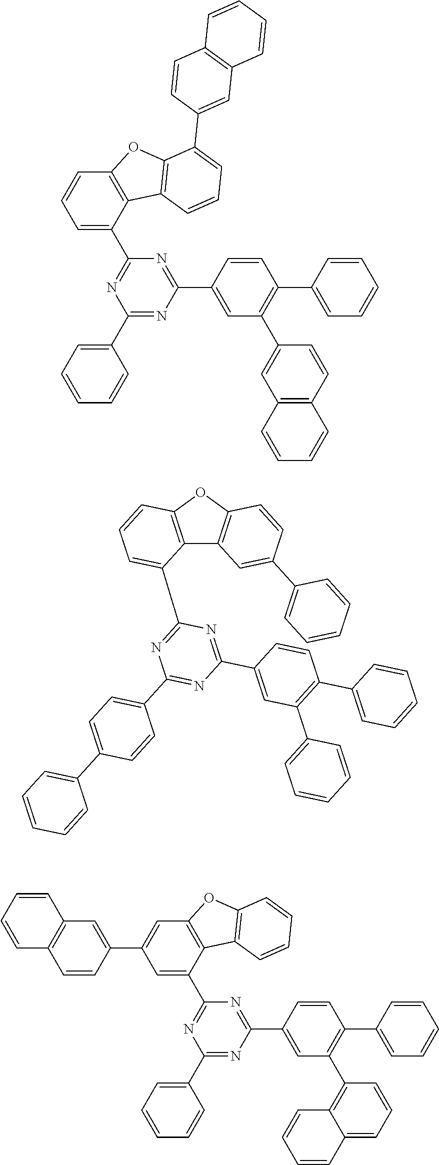 [SMILES: c1ccc(-c2nc(-c3ccc(-c4ccccc4)c(-c4ccc5ccccc5c4)c3)nc(-c3cccc4oc5c(-c6ccc7ccccc7c6)cccc5c34)n2)cc1 ; c1ccc(-c2ccc(-c3nc(-c4ccc(-c5ccccc5)c(-c5ccccc5)c4)nc(-c4cccc5oc6ccc(-c7ccccc7)cc6c45)n3)cc2)cc1 ; c1ccc(-c2nc(-c3ccc(-c4ccccc4)c(-c4cccc5ccccc45)c3)nc(-c3cc(-c4ccc5ccccc5c4)cc4oc5ccccc5c34)n2)cc1]

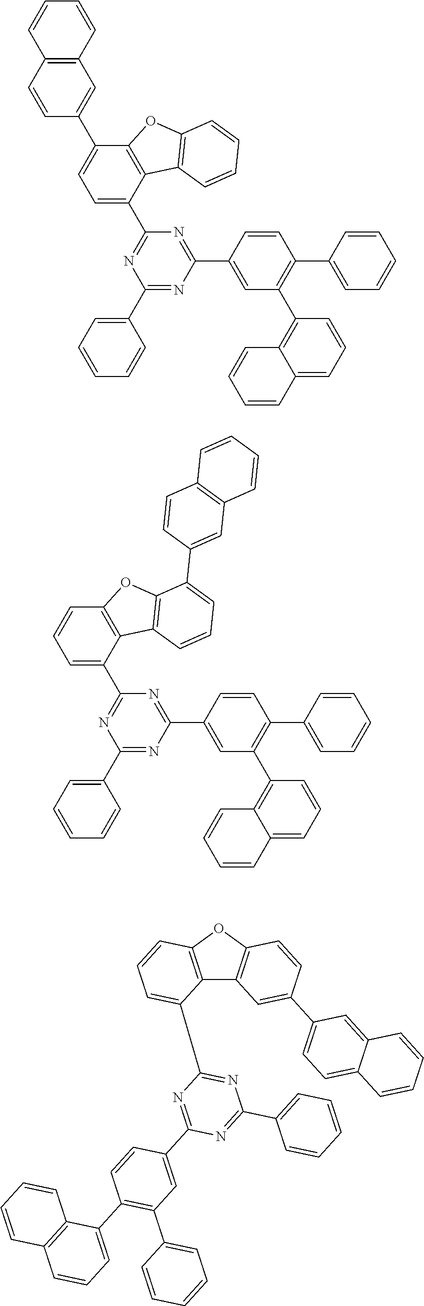

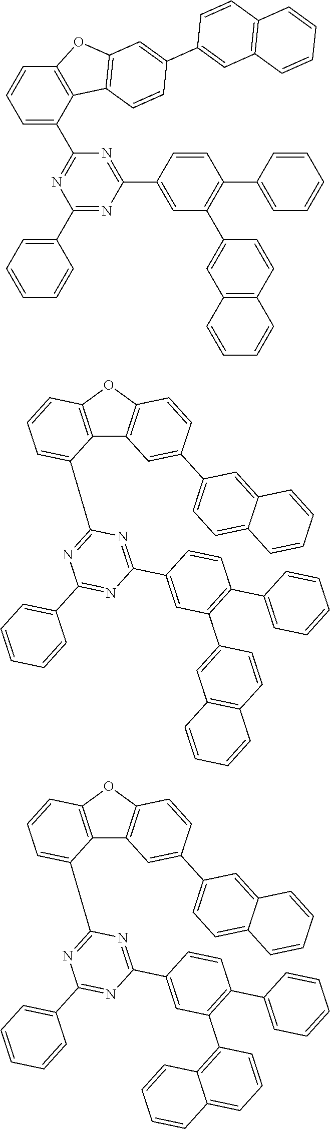 [SMILES: c1ccc(-c2nc(-c3ccc(-c4ccccc4)c(-c4ccc5ccccc5c4)c3)nc(-c3cccc4oc5cc(-c6ccc7ccccc7c6)ccc5c34)n2)cc1 ; c1ccc(-c2nc(-c3ccc(-c4ccccc4)c(-c4ccc5ccccc5c4)c3)nc(-c3cccc4oc5ccc(-c6ccc7ccccc7c6)cc5c34)n2)cc1 ; c1ccc(-c2nc(-c3ccc(-c4ccccc4)c(-c4cccc5ccccc45)c3)nc(-c3cccc4oc5ccc(-c6ccc7ccccc7c6)cc5c34)n2)cc1]

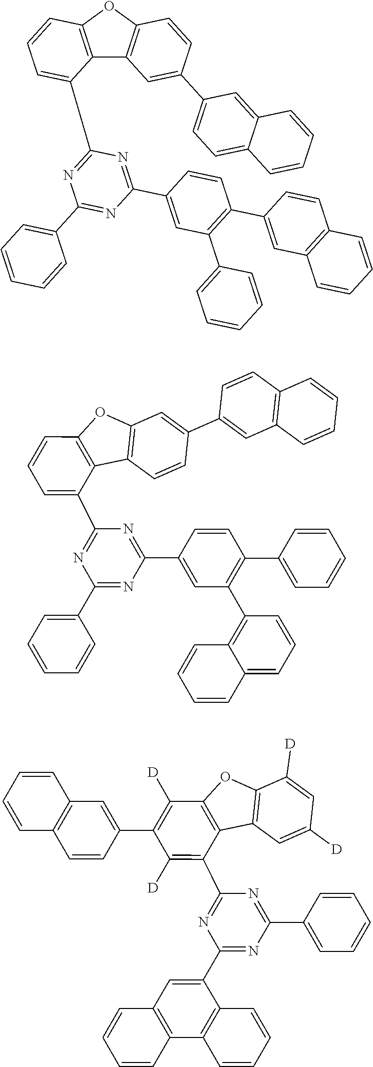

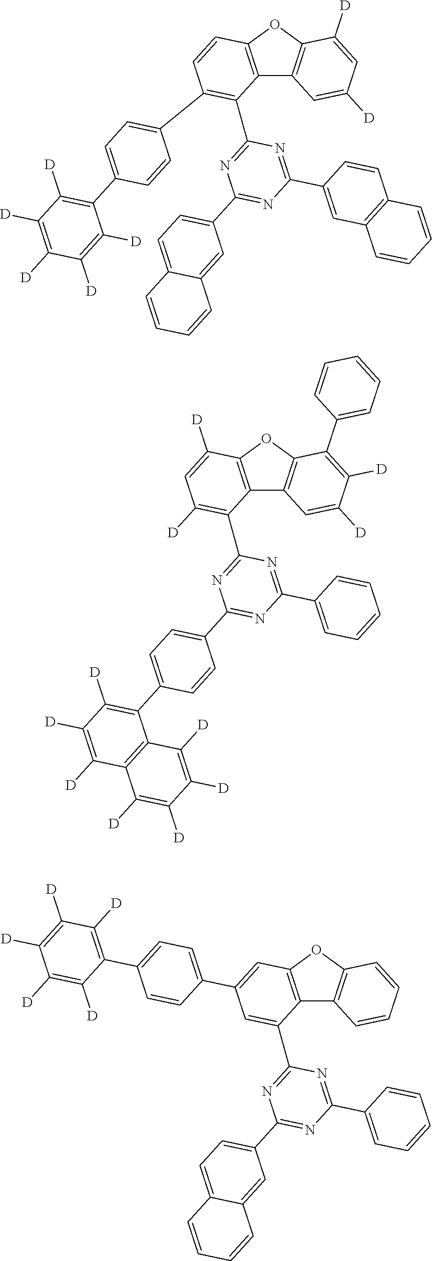

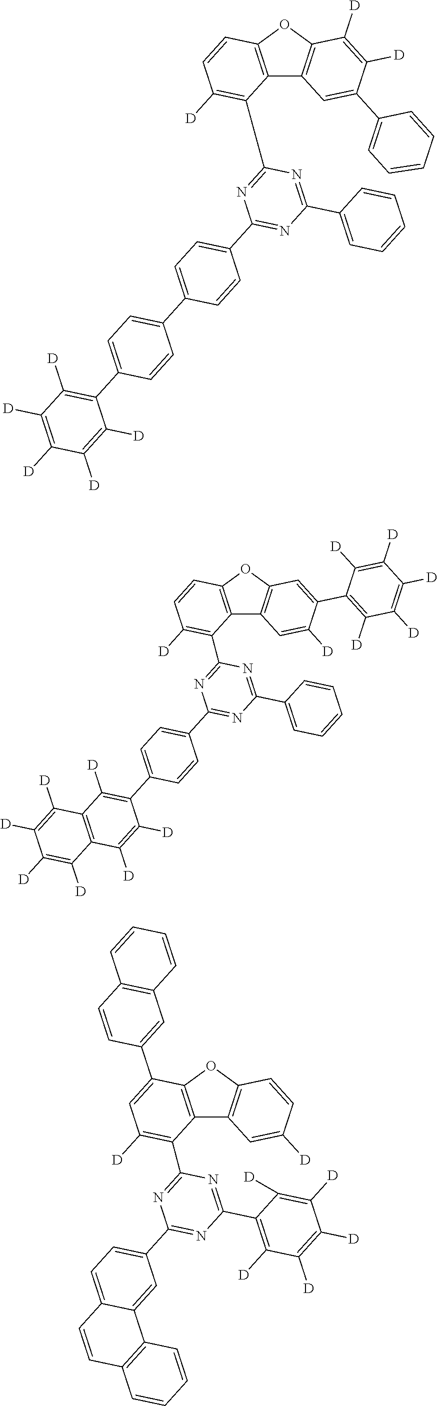 [SMILES: [2H]c1ccc2oc3c([2H])c([2H])c(-c4ccccc4)cc3c2c1-c1nc(-c2ccccc2)nc(-c2ccc(-c3ccc(-c4c([2H])c([2H])c([2H])c([2H])c4[2H])cc3)cc2)n1 ; [2H]c1cc2c(cc1-c1c([2H])c([2H])c([2H])c([2H])c1[2H])oc1ccc([2H])c(-c3nc(-c4ccccc4)nc(-c4ccc(-c5c([2H])c([2H])c6c([2H])c([2H])c([2H])c([2H])c6c5[2H])cc4)n3)c12 ; [2H]c1ccc2oc3c(-c4ccc5ccccc5c4)cc([2H])c(-c4nc(-c5ccc6ccc7ccccc7c6c5)nc(-c5c([2H])c([2H])c([2H])c([2H])c5[2H])n4)c3c2c1]

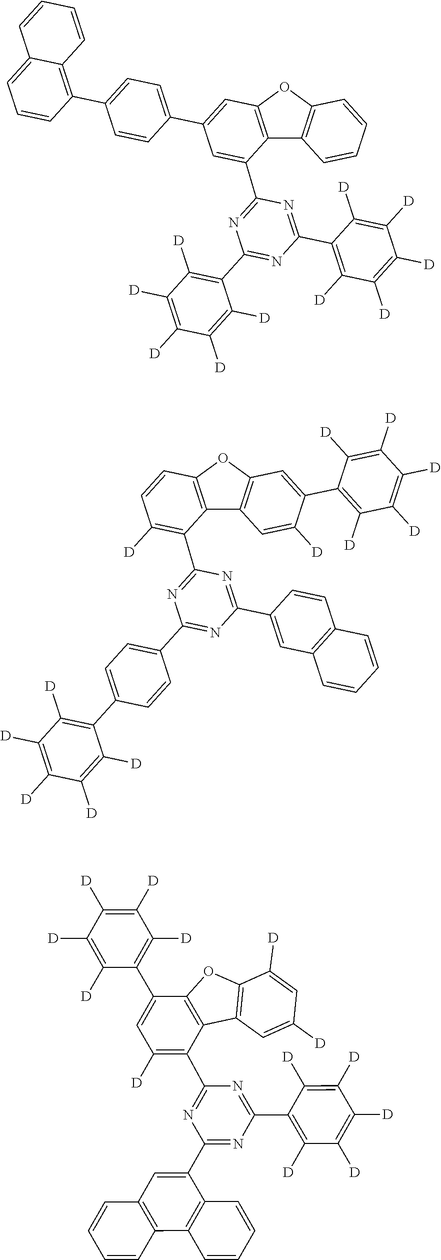 [SMILES: [2H]c1c([2H])c([2H])c(-c2nc(-c3c([2H])c([2H])c([2H])c([2H])c3[2H])nc(-c3cc(-c4ccc(-c5cccc6ccccc56)cc4)cc4oc5ccccc5c34)n2)c([2H])c1[2H] ; [2H]c1cc2c(cc1-c1c([2H])c([2H])c([2H])c([2H])c1[2H])oc1ccc([2H])c(-c3nc(-c4ccc(-c5c([2H])c([2H])c([2H])c([2H])c5[2H])cc4)nc(-c4ccc5ccccc5c4)n3)c12 ; [2H]c1cc([2H])c2oc3c(-c4c([2H])c([2H])c([2H])c([2H])c4[2H])cc([2H])c(-c4nc(-c5c([2H])c([2H])c([2H])c([2H])c5[2H])nc(-c5cc6ccccc6c6ccccc56)n4)c3c2c1]

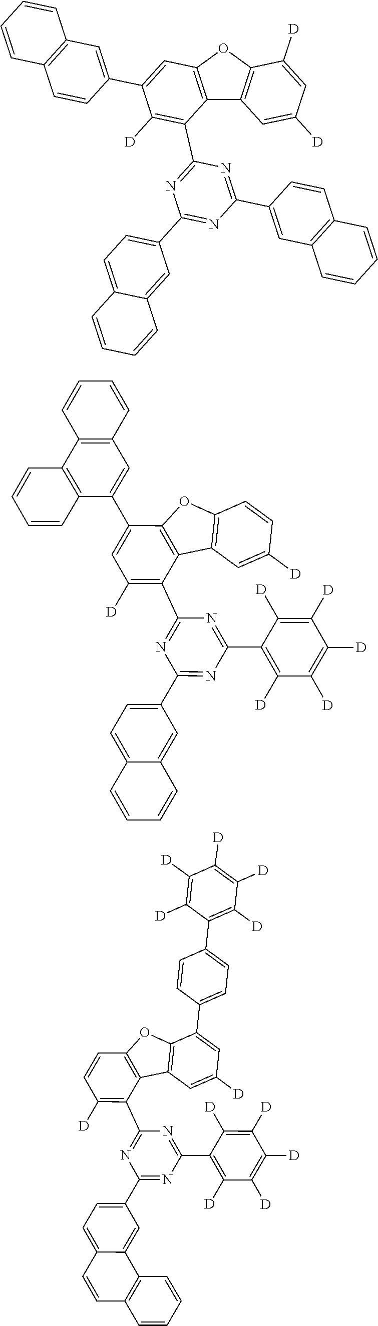

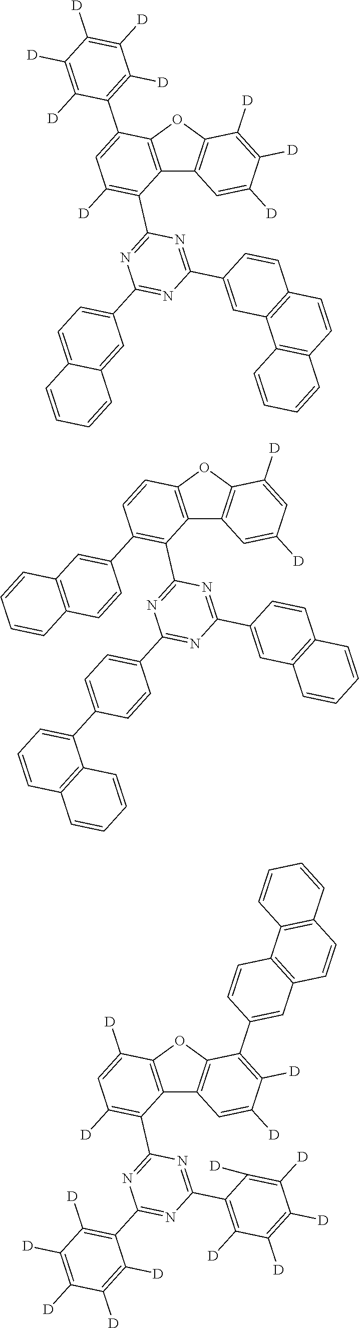

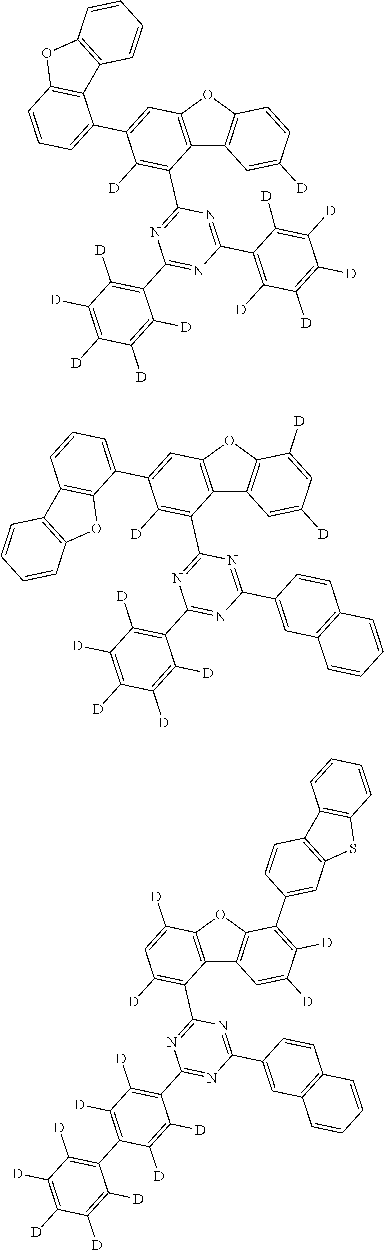 [SMILES: [2H]c1ccc2oc3cc(-c4cccc5oc6ccccc6c45)c([2H])c(-c4nc(-c5c([2H])c([2H])c([2H])c([2H])c5[2H])nc(-c5c([2H])c([2H])c([2H])c([2H])c5[2H])n4)c3c2c1 ; [2H]c1cc([2H])c2oc3cc(-c4cccc5c4oc4ccccc45)c([2H])c(-c4nc(-c5ccc6ccccc6c5)nc(-c5c([2H])c([2H])c([2H])c([2H])c5[2H])n4)c3c2c1 ; [2H]c1cc2c(oc3c([2H])cc([2H])c(-c4nc(-c5ccc6ccccc6c5)nc(-c5c([2H])c([2H])c(-c6c([2H])c([2H])c([2H])c([2H])c6[2H])c([2H])c5[2H])n4)c32)c(-c2ccc3c(c2)sc2ccccc23)c1[2H]]

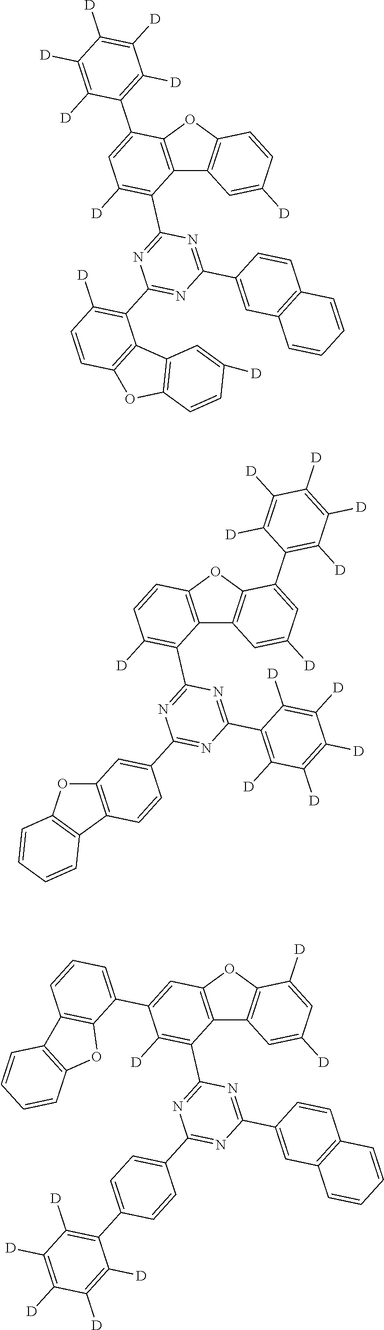 [SMILES: [2H]c1ccc2oc3ccc([2H])c(-c4nc(-c5ccc6ccccc6c5)nc(-c5c([2H])cc(-c6c([2H])c([2H])c([2H])c([2H])c6[2H])c6oc7ccc([2H])cc7c56)n4)c3c2c1 ; [2H]c1cc(-c2c([2H])c([2H])c([2H])c([2H])c2[2H])c2oc3ccc([2H])c(-c4nc(-c5ccc6c(c5)oc5ccccc56)nc(-c5c([2H])c([2H])c([2H])c([2H])c5[2H])n4)c3c2c1 ; [2H]c1cc([2H])c2oc3cc(-c4cccc5c4oc4ccccc45)c([2H])c(-c4nc(-c5ccc(-c6c([2H])c([2H])c([2H])c([2H])c6[2H])cc5)nc(-c5ccc6ccccc6c5)n4)c3c2c1]

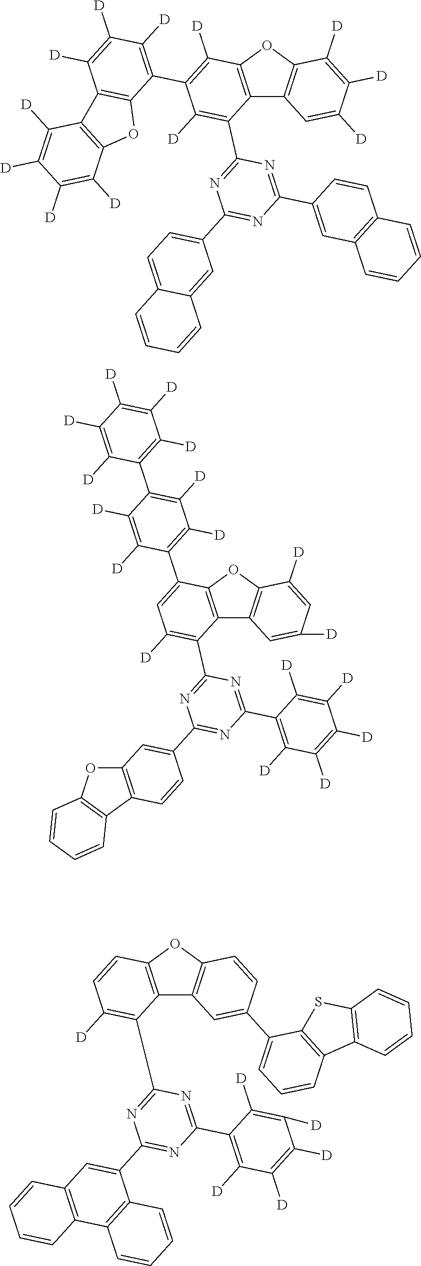

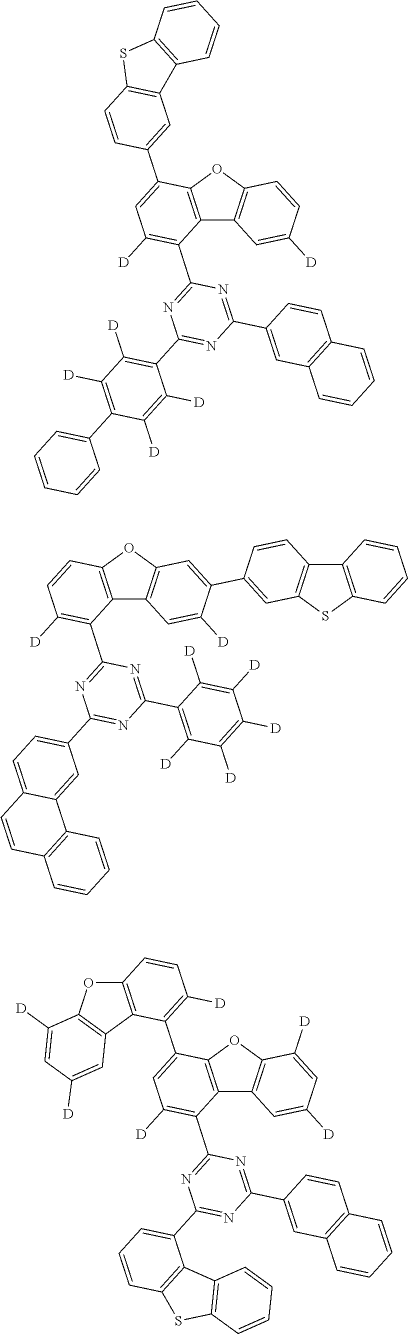 [SMILES: [2H]c1ccc2oc3c(-c4ccc5sc6ccccc6c5c4)cc([2H])c(-c4nc(-c5ccc6ccccc6c5)nc(-c5c([2H])c([2H])c(-c6ccccc6)c([2H])c5[2H])n4)c3c2c1 ; [2H]c1cc2c(cc1-c1ccc3c(c1)sc1ccccc13)oc1ccc([2H])c(-c3nc(-c4ccc5ccc6ccccc6c5c4)nc(-c4c([2H])c([2H])c([2H])c([2H])c4[2H])n3)c12 ; [2H]c1cc([2H])c2oc3ccc([2H])c(-c4cc([2H])c(-c5nc(-c6ccc7ccccc7c6)nc(-c6cccc7sc8ccccc8c67)n5)c5c4oc4c([2H])cc([2H])cc45)c3c2c1]

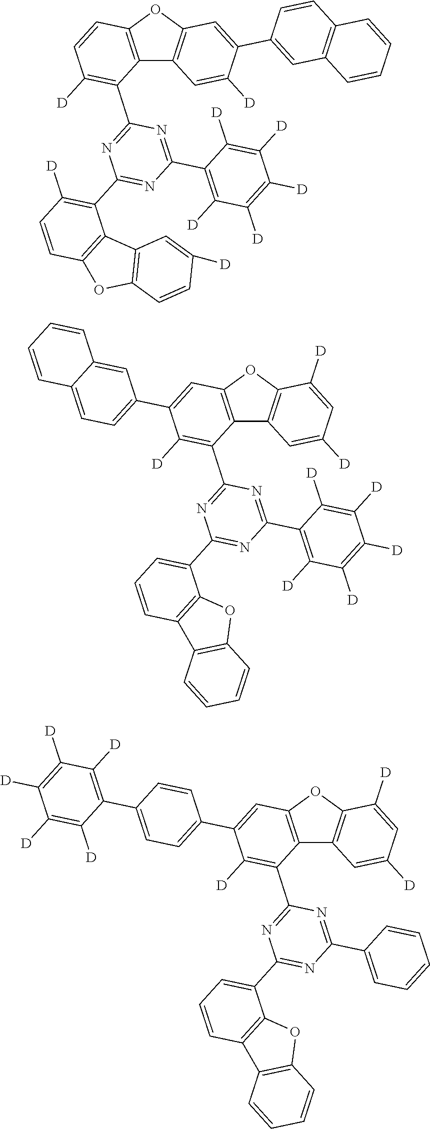

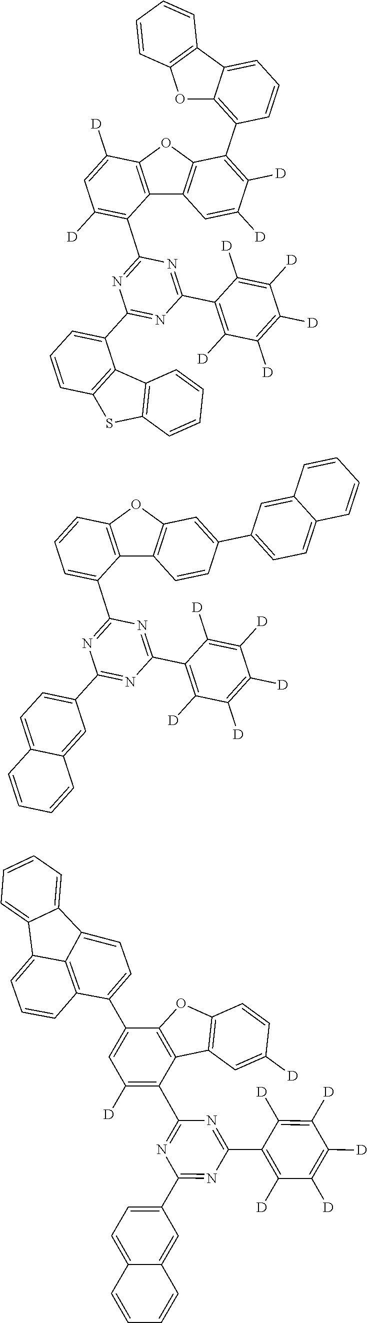

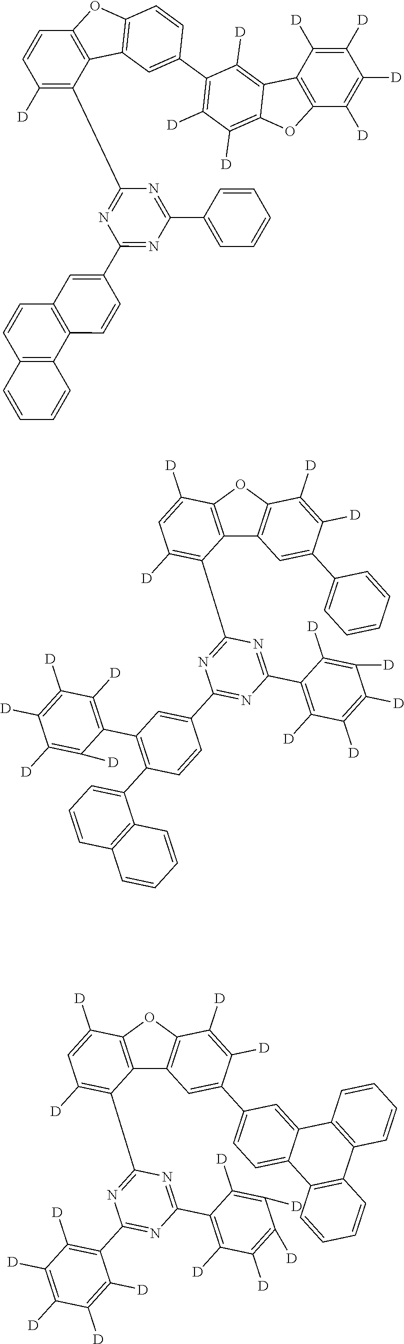 [SMILES: [2H]c1ccc2oc3ccc(-c4c([2H])c([2H])c5oc6c([2H])c([2H])c([2H])c([2H])c6c5c4[2H])cc3c2c1-c1nc(-c2ccccc2)nc(-c2ccc3c(ccc4ccccc43)c2)n1 ; [2H]c1cc([2H])c2oc3c([2H])c([2H])c(-c4ccccc4)cc3c2c1-c1nc(-c2ccc(-c3cccc4ccccc34)c(-c3c([2H])c([2H])c([2H])c([2H])c3[2H])c2)nc(-c2c([2H])c([2H])c([2H])c([2H])c2[2H])n1 ; [2H]c1cc([2H])c2oc3c([2H])c([2H])c(-c4ccc5c6ccccc6c6ccccc6c5c4)cc3c2c1-c1nc(-c2c([2H])c([2H])c([2H])c([2H])c2[2H])nc(-c2c([2H])c([2H])c([2H])c([2H])c2[2H])n1]

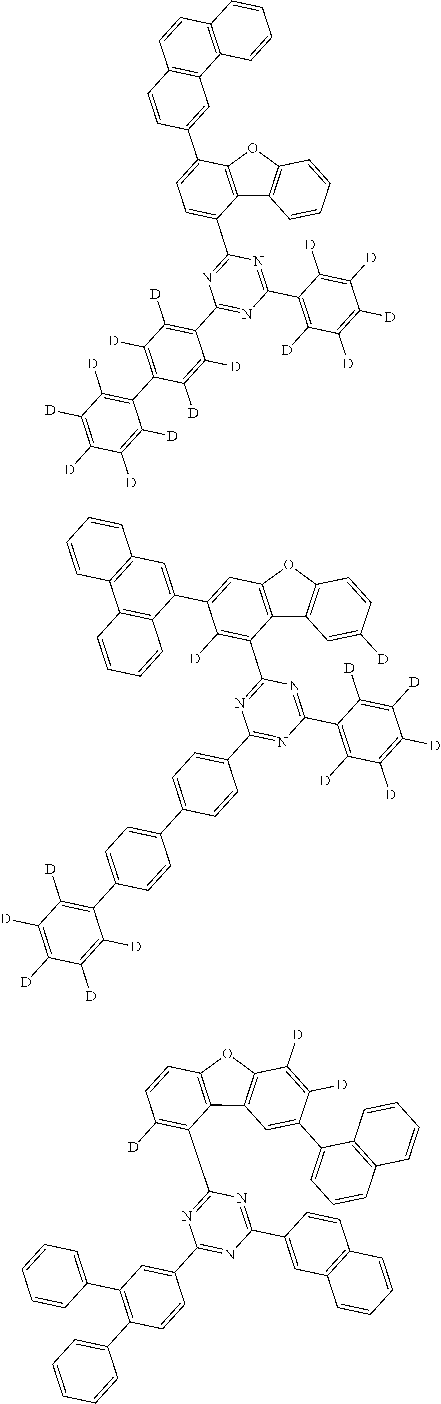

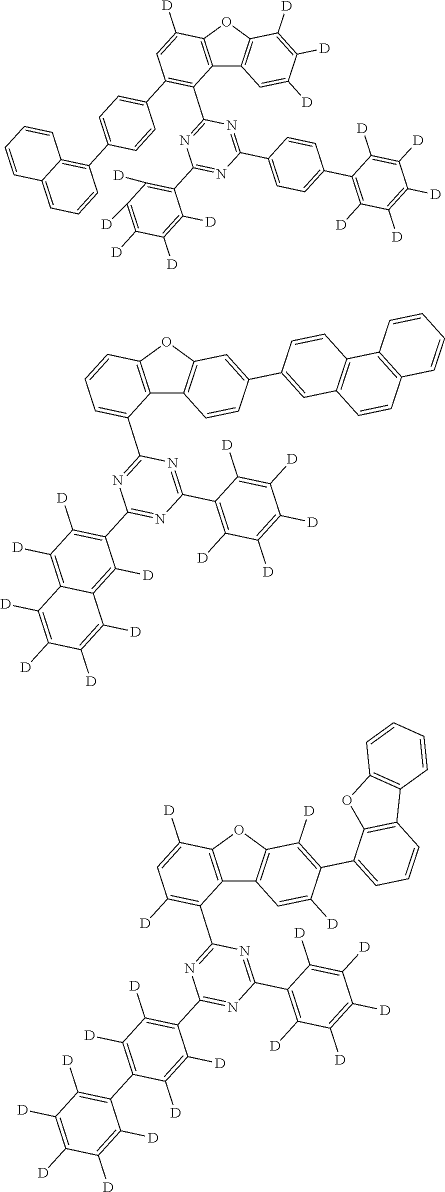

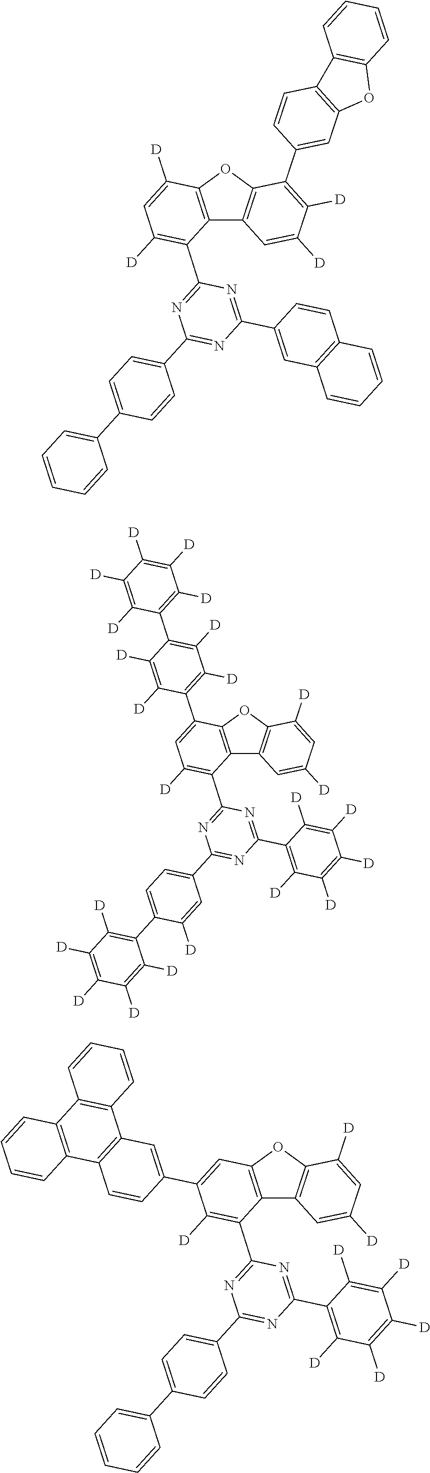

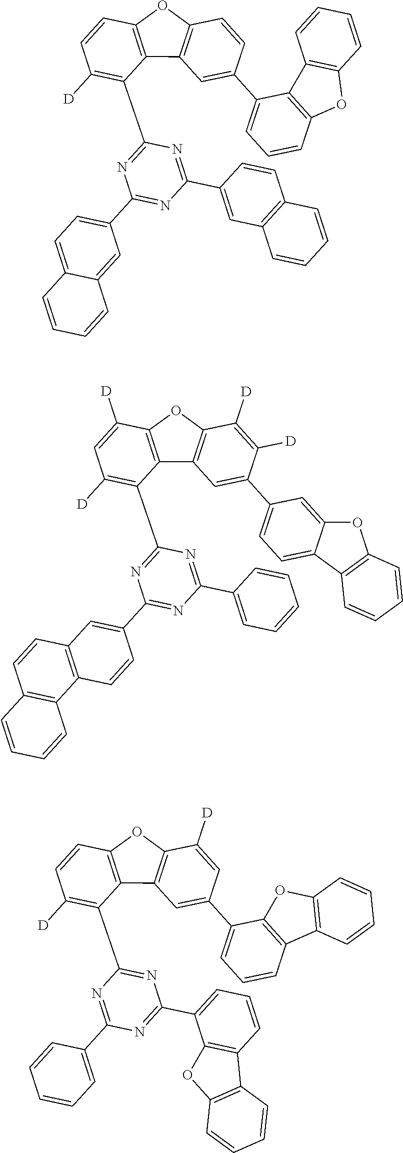 [SMILES: [2H]c1ccc2oc3ccc(-c4cccc5oc6ccccc6c45)cc3c2c1-c1nc(-c2ccc3ccccc3c2)nc(-c2ccc3ccccc3c2)n1 ; [2H]c1cc([2H])c2oc3c([2H])c([2H])c(-c4ccc5c(c4)oc4ccccc45)cc3c2c1-c1nc(-c2ccccc2)nc(-c2ccc3c(ccc4ccccc43)c2)n1 ; [2H]c1ccc2oc3c([2H])cc(-c4cccc5c4oc4ccccc45)cc3c2c1-c1nc(-c2ccccc2)nc(-c2cccc3c2oc2ccccc23)n1]

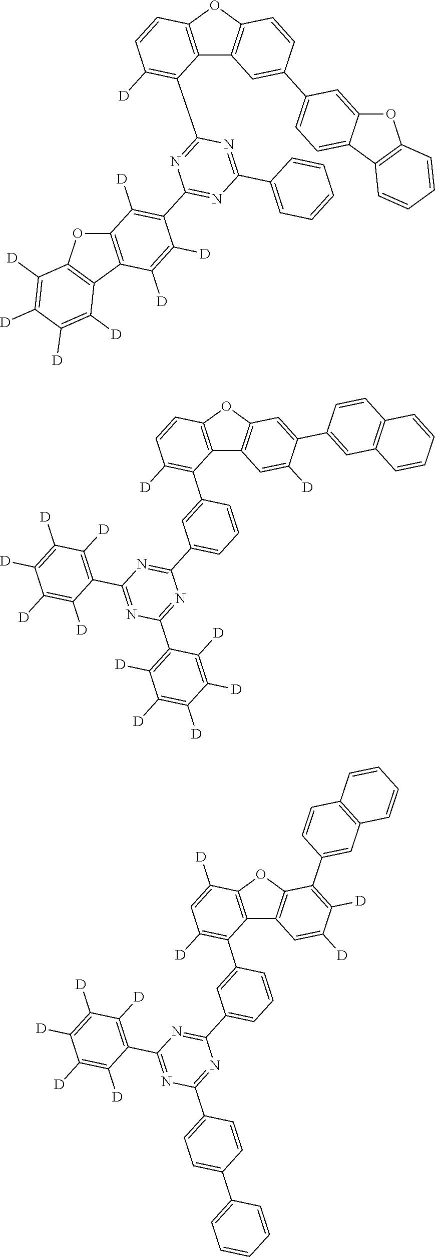 [SMILES: [2H]c1ccc2oc3ccc(-c4ccc5c(c4)oc4ccccc45)cc3c2c1-c1nc(-c2ccccc2)nc(-c2c([2H])c([2H])c3c(oc4c([2H])c([2H])c([2H])c([2H])c43)c2[2H])n1 ; [2H]c1cc2c(cc1-c1ccc3ccccc3c1)oc1ccc([2H])c(-c3cccc(-c4nc(-c5c([2H])c([2H])c([2H])c([2H])c5[2H])nc(-c5c([2H])c([2H])c([2H])c([2H])c5[2H])n4)c3)c12 ; [2H]c1cc2c(oc3c([2H])cc([2H])c(-c4cccc(-c5nc(-c6ccc(-c7ccccc7)cc6)nc(-c6c([2H])c([2H])c([2H])c([2H])c6[2H])n5)c4)c32)c(-c2ccc3ccccc3c2)c1[2H]]

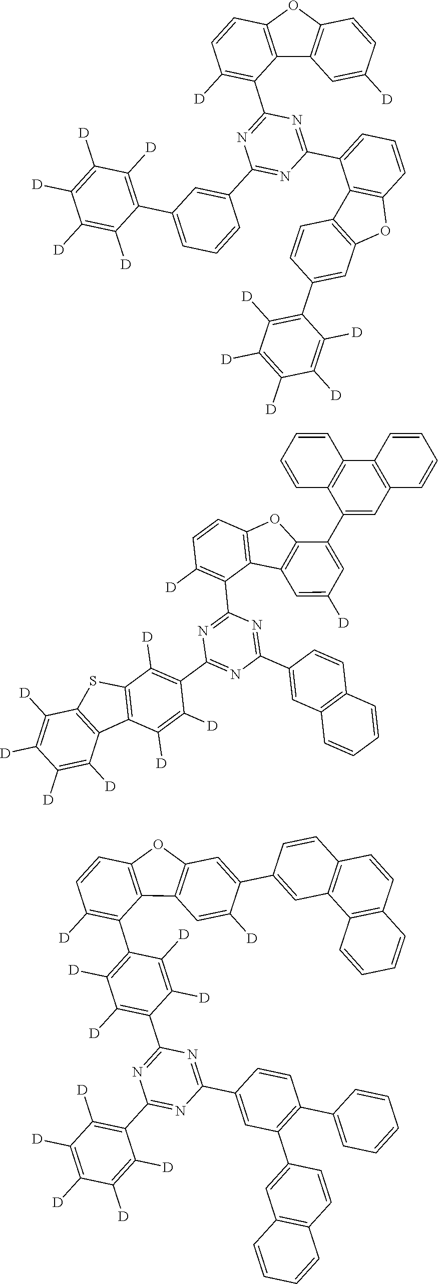

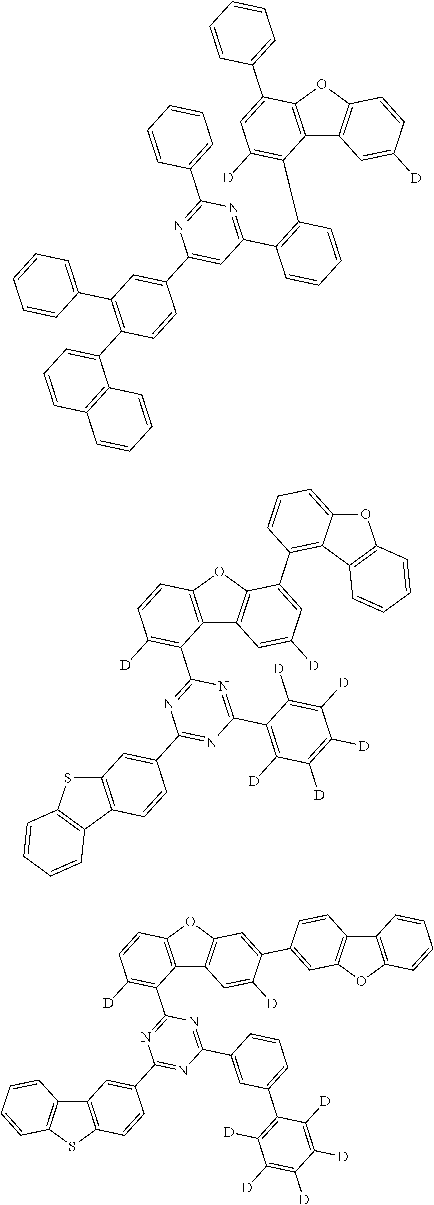 [SMILES: [2H]c1ccc2oc3c(-c4ccccc4)cc([2H])c(-c4ccccc4-c4cc(-c5ccc(-c6cccc7ccccc67)c(-c6ccccc6)c5)nc(-c5ccccc5)n4)c3c2c1 ; [2H]c1cc(-c2cccc3oc4ccccc4c23)c2oc3ccc([2H])c(-c4nc(-c5ccc6c(c5)sc5ccccc56)nc(-c5c([2H])c([2H])c([2H])c([2H])c5[2H])n4)c3c2c1 ; [2H]c1cc2c(cc1-c1ccc3c(c1)oc1ccccc13)oc1ccc([2H])c(-c3nc(-c4cccc(-c5c([2H])c([2H])c([2H])c([2H])c5[2H])c4)nc(-c4ccc5sc6ccccc6c5c4)n3)c12]

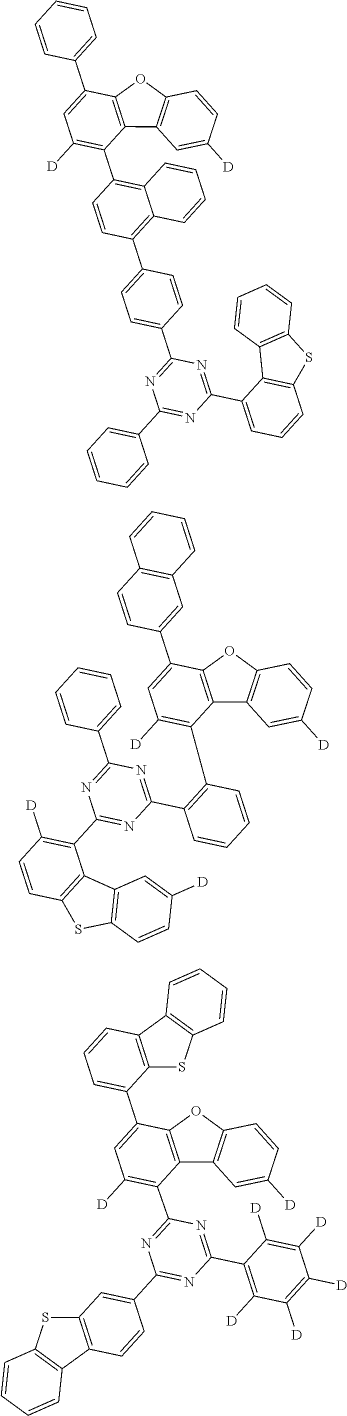

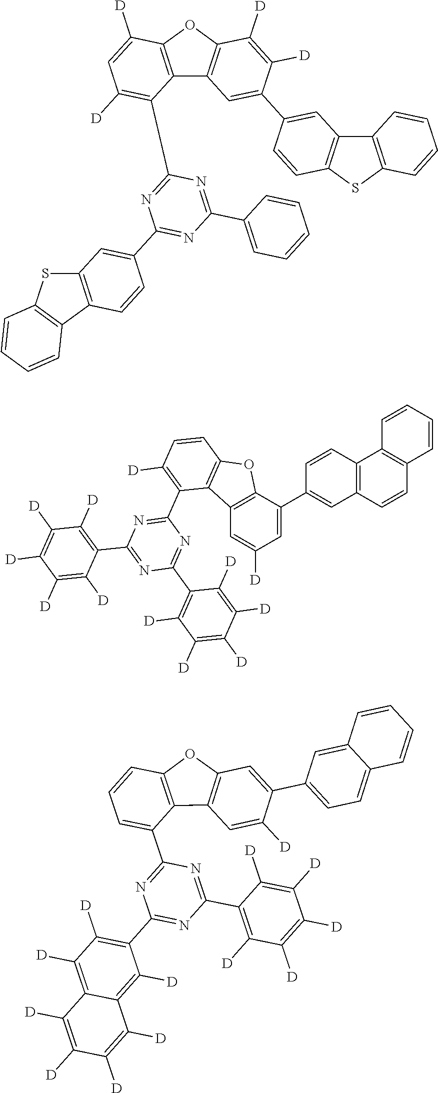 [SMILES: [2H]c1cc([2H])c2oc3c([2H])c([2H])c(-c4ccc5sc6ccccc6c5c4)cc3c2c1-c1nc(-c2ccccc2)nc(-c2ccc3c(c2)sc2ccccc23)n1 ; [2H]c1cc(-c2ccc3c(ccc4ccccc43)c2)c2oc3ccc([2H])c(-c4nc(-c5c([2H])c([2H])c([2H])c([2H])c5[2H])nc(-c5c([2H])c([2H])c([2H])c([2H])c5[2H])n4)c3c2c1 ; [2H]c1cc2c(cc1-c1ccc3ccccc3c1)oc1cccc(-c3nc(-c4c([2H])c([2H])c([2H])c([2H])c4[2H])nc(-c4c([2H])c([2H])c5c([2H])c([2H])c([2H])c([2H])c5c4[2H])n3)c12]

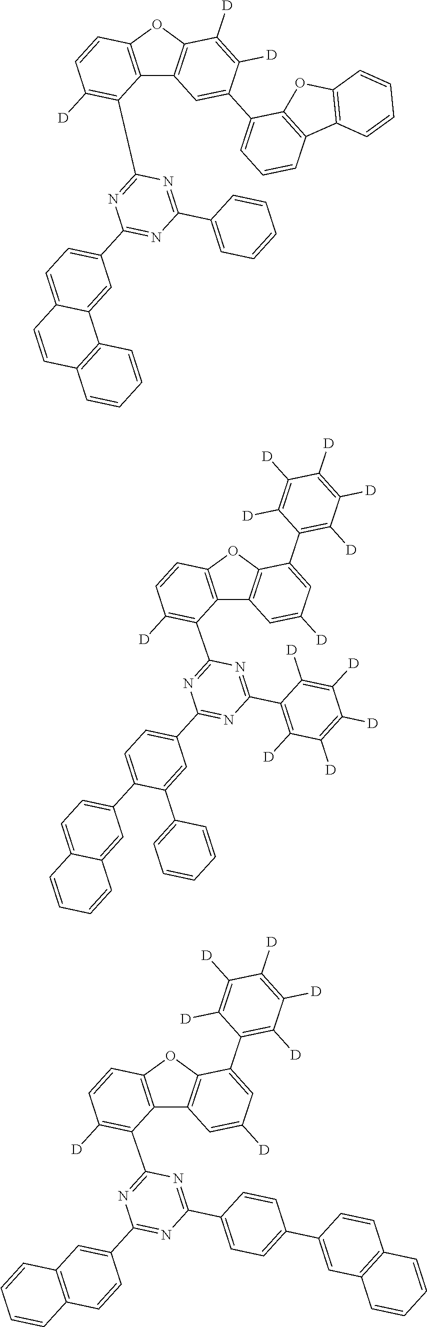 [SMILES: [2H]c1ccc2oc3c([2H])c([2H])c(-c4cccc5c4oc4ccccc45)cc3c2c1-c1nc(-c2ccccc2)nc(-c2ccc3ccc4ccccc4c3c2)n1 ; [2H]c1cc(-c2c([2H])c([2H])c([2H])c([2H])c2[2H])c2oc3ccc([2H])c(-c4nc(-c5ccc(-c6ccc7ccccc7c6)c(-c6ccccc6)c5)nc(-c5c([2H])c([2H])c([2H])c([2H])c5[2H])n4)c3c2c1 ; [2H]c1cc(-c2c([2H])c([2H])c([2H])c([2H])c2[2H])c2oc3ccc([2H])c(-c4nc(-c5ccc(-c6ccc7ccccc7c6)cc5)nc(-c5ccc6ccccc6c5)n4)c3c2c1]

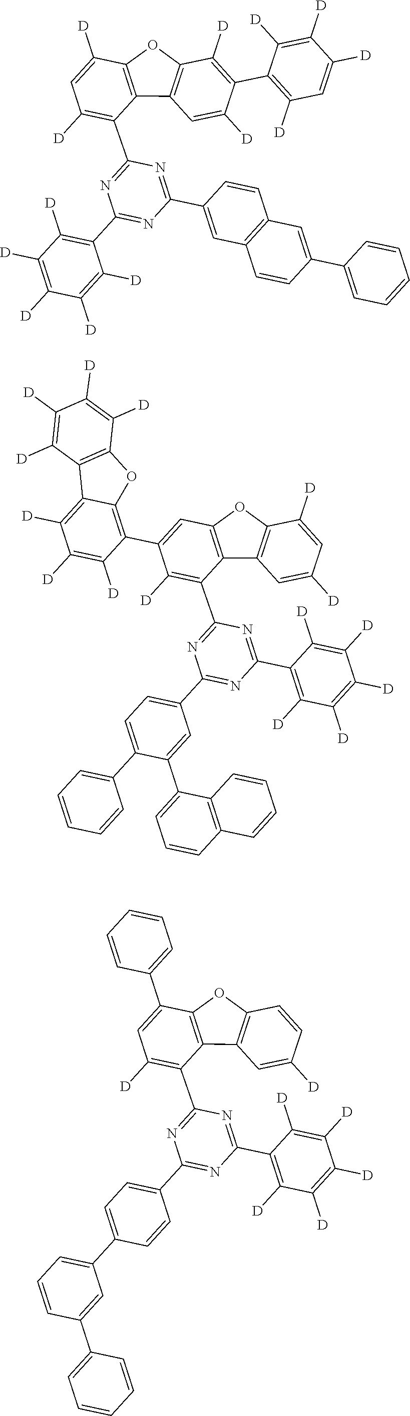

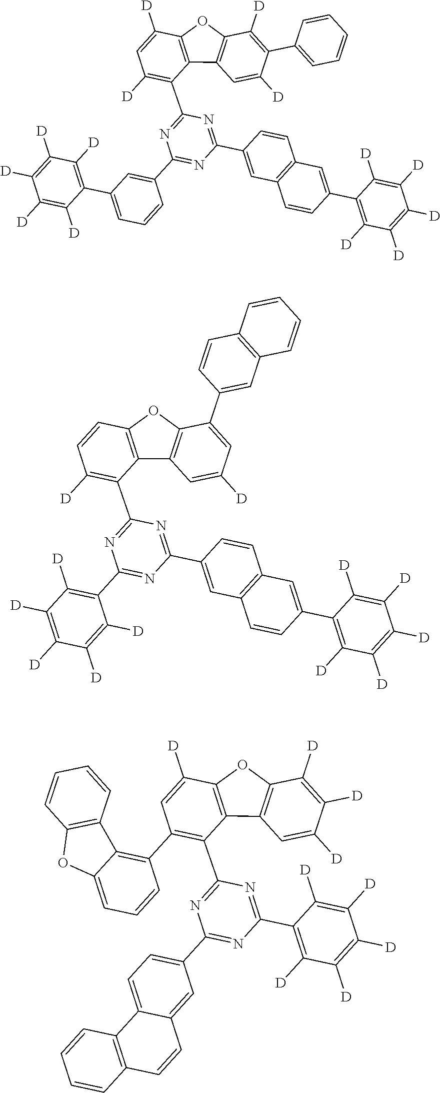 [SMILES: [2H]c1cc2c(oc3c([2H])cc([2H])c(-c4nc(-c5cccc(-c6c([2H])c([2H])c([2H])c([2H])c6[2H])c5)nc(-c5ccc6cc(-c7c([2H])c([2H])c([2H])c([2H])c7[2H])ccc6c5)n4)c32)c([2H])c1-c1ccccc1 ; [2H]c1cc(-c2ccc3ccccc3c2)c2oc3ccc([2H])c(-c4nc(-c5ccc6cc(-c7c([2H])c([2H])c([2H])c([2H])c7[2H])ccc6c5)nc(-c5c([2H])c([2H])c([2H])c([2H])c5[2H])n4)c3c2c1 ; [2H]c1cc2c(oc3c([2H])cc(-c4cccc5oc6ccccc6c45)c(-c4nc(-c5ccc6c(ccc7ccccc76)c5)nc(-c5c([2H])c([2H])c([2H])c([2H])c5[2H])n4)c32)c([2H])c1[2H]]

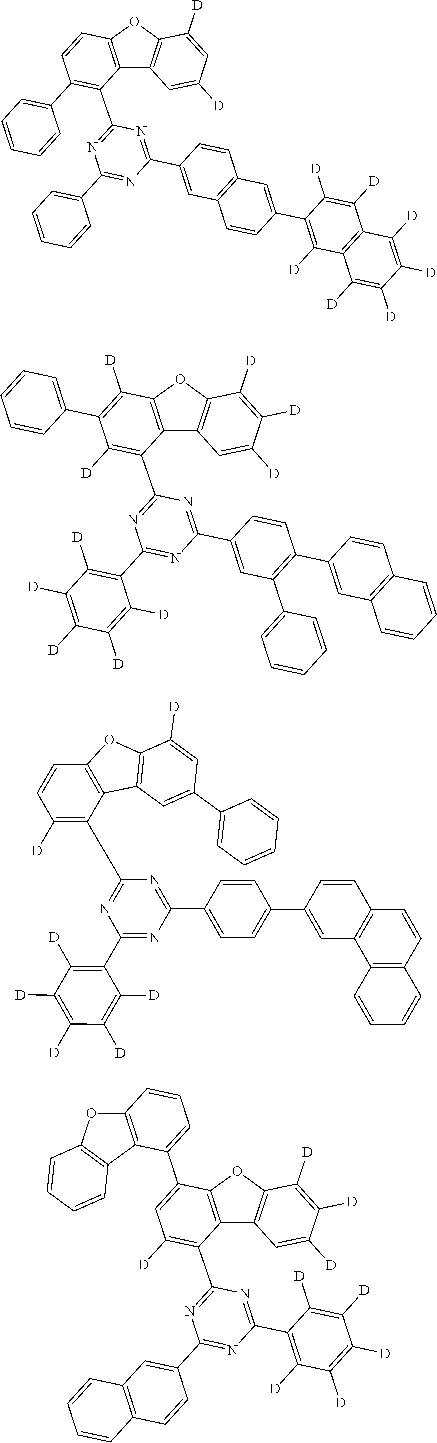

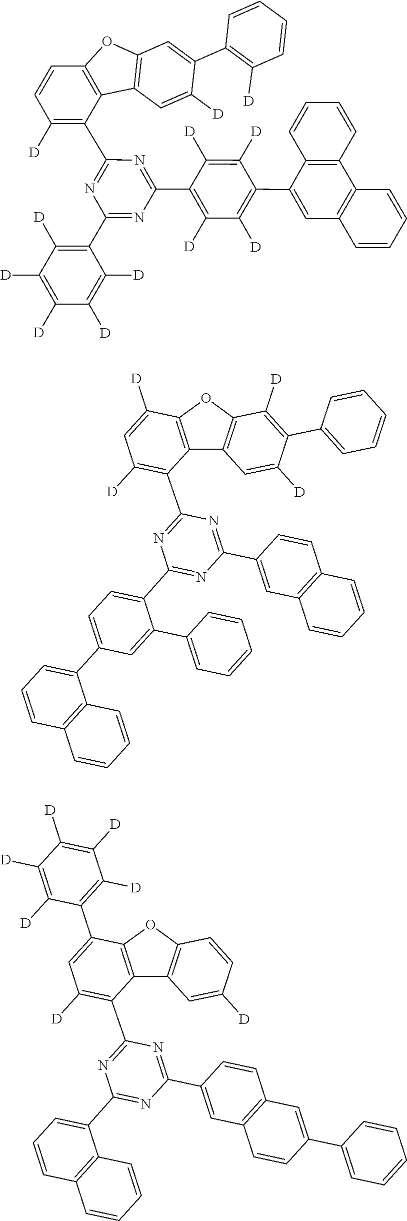 [SMILES: [2H]c1ccccc1-c1cc2oc3ccc([2H])c(-c4nc(-c5c([2H])c([2H])c([2H])c([2H])c5[2H])nc(-c5c([2H])c([2H])c(-c6cc7ccccc7c7ccccc67)c([2H])c5[2H])n4)c3c2cc1[2H] ; [2H]c1cc2c(oc3c([2H])cc([2H])c(-c4nc(-c5ccc6ccccc6c5)nc(-c5ccc(-c6cccc7ccccc67)cc5-c5ccccc5)n4)c32)c([2H])c1-c1ccccc1 ; [2H]c1ccc2oc3c(-c4c([2H])c([2H])c([2H])c([2H])c4[2H])cc([2H])c(-c4nc(-c5ccc6cc(-c7ccccc7)ccc6c5)nc(-c5cccc6ccccc56)n4)c3c2c1]

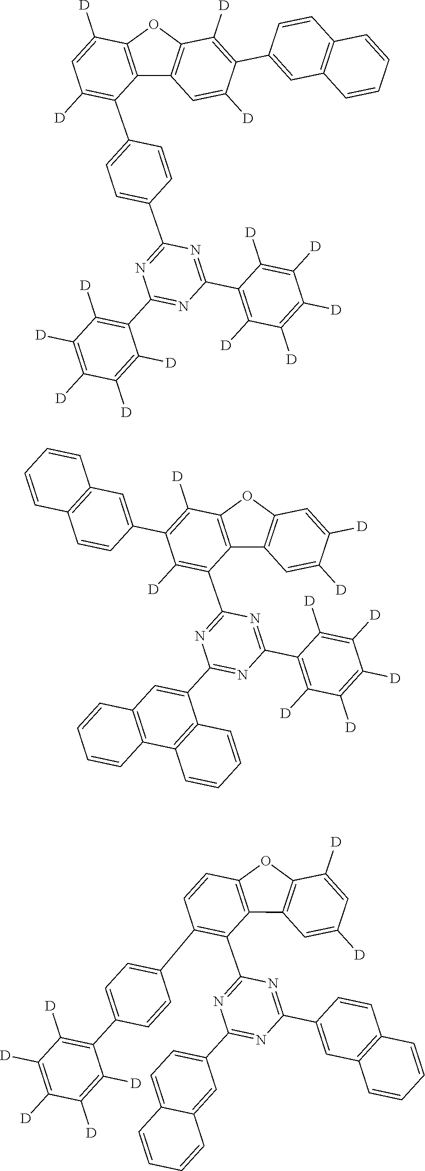 [SMILES: [2H]c1cc2c(oc3c([2H])cc([2H])c(-c4ccc(-c5nc(-c6c([2H])c([2H])c([2H])c([2H])c6[2H])nc(-c6c([2H])c([2H])c([2H])c([2H])c6[2H])n5)cc4)c32)c([2H])c1-c1ccc2ccccc2c1 ; [2H]c1cc2oc3c([2H])c(-c4ccc5ccccc5c4)c([2H])c(-c4nc(-c5c([2H])c([2H])c([2H])c([2H])c5[2H])nc(-c5cc6ccccc6c6ccccc56)n4)c3c2cc1[2H] ; [2H]c1cc([2H])c2oc3ccc(-c4ccc(-c5c([2H])c([2H])c([2H])c([2H])c5[2H])cc4)c(-c4nc(-c5ccc6ccccc6c5)nc(-c5ccc6ccccc6c5)n4)c3c2c1]

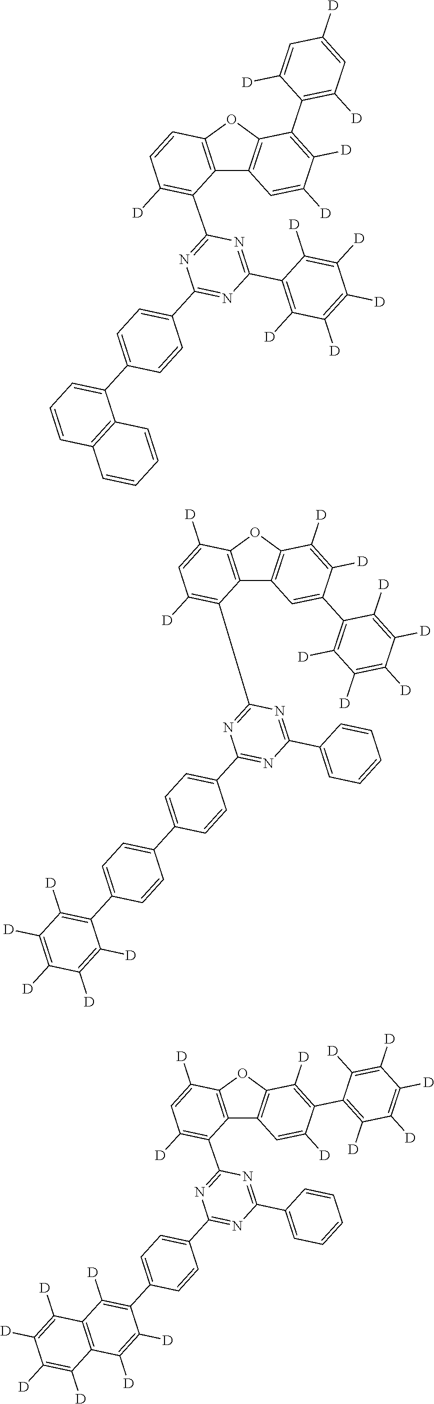 [SMILES: [2H]c1cc([2H])c(-c2c([2H])c([2H])cc3c2oc2ccc([2H])c(-c4nc(-c5ccc(-c6cccc7ccccc67)cc5)nc(-c5c([2H])c([2H])c([2H])c([2H])c5[2H])n4)c23)c([2H])c1 ; [2H]c1cc([2H])c2oc3c([2H])c([2H])c(-c4c([2H])c([2H])c([2H])c([2H])c4[2H])cc3c2c1-c1nc(-c2ccccc2)nc(-c2ccc(-c3ccc(-c4c([2H])c([2H])c([2H])c([2H])c4[2H])cc3)cc2)n1 ; [2H]c1cc2c(oc3c([2H])cc([2H])c(-c4nc(-c5ccccc5)nc(-c5ccc(-c6c([2H])c([2H])c7c([2H])c([2H])c([2H])c([2H])c7c6[2H])cc5)n4)c32)c([2H])c1-c1c([2H])c([2H])c([2H])c([2H])c1[2H]]

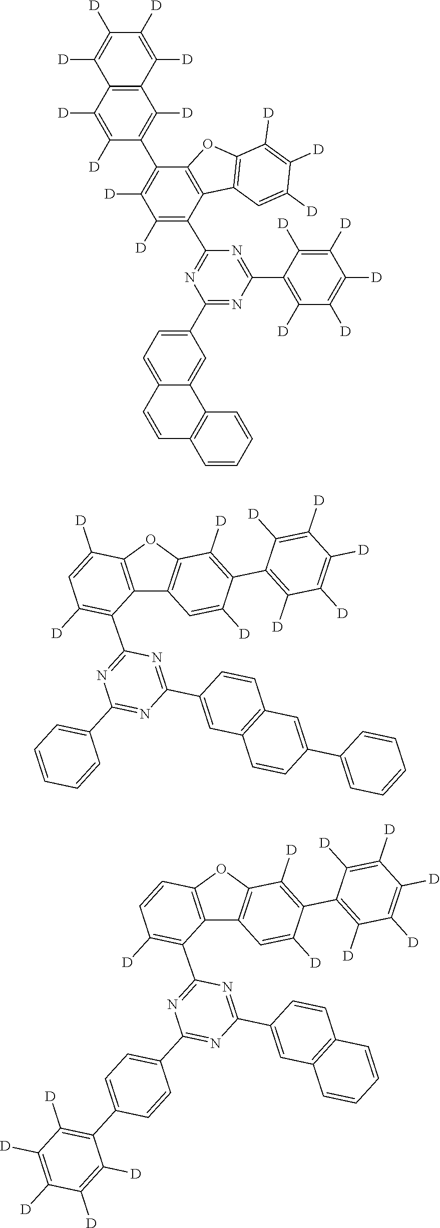

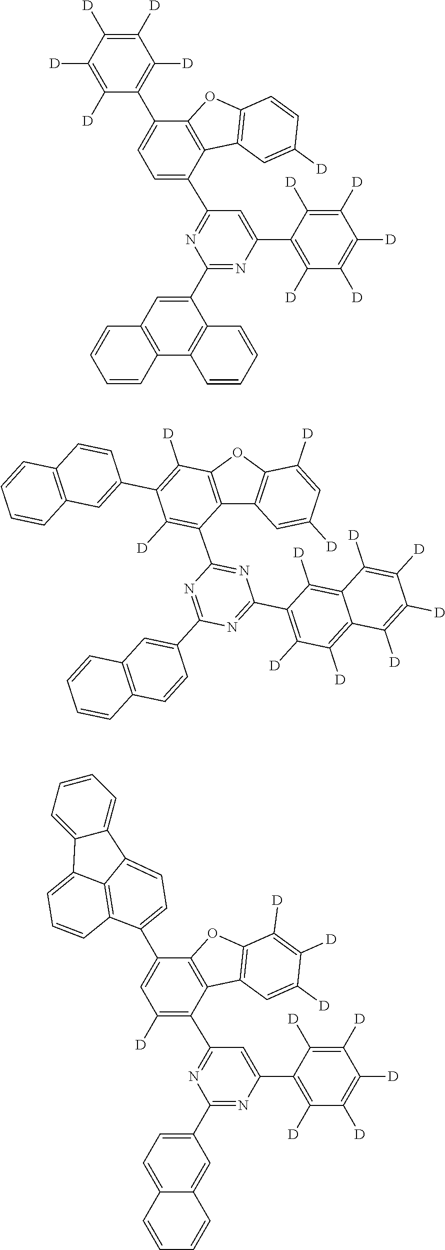 [SMILES: [2H]c1ccc2oc3c(-c4c([2H])c([2H])c([2H])c([2H])c4[2H])ccc(-c4cc(-c5c([2H])c([2H])c([2H])c([2H])c5[2H])nc(-c5cc6ccccc6c6ccccc56)n4)c3c2c1 ; [2H]c1cc([2H])c2oc3c([2H])c(-c4ccc5ccccc5c4)c([2H])c(-c4nc(-c5ccc6ccccc6c5)nc(-c5c([2H])c([2H])c6c([2H])c([2H])c([2H])c([2H])c6c5[2H])n4)c3c2c1 ; [2H]c1cc2c(oc3c(-c4ccc5c6c(cccc46)-c4ccccc4-5)cc([2H])c(-c4cc(-c5c([2H])c([2H])c([2H])c([2H])c5[2H])nc(-c5ccc6ccccc6c5)n4)c32)c([2H])c1[2H]]

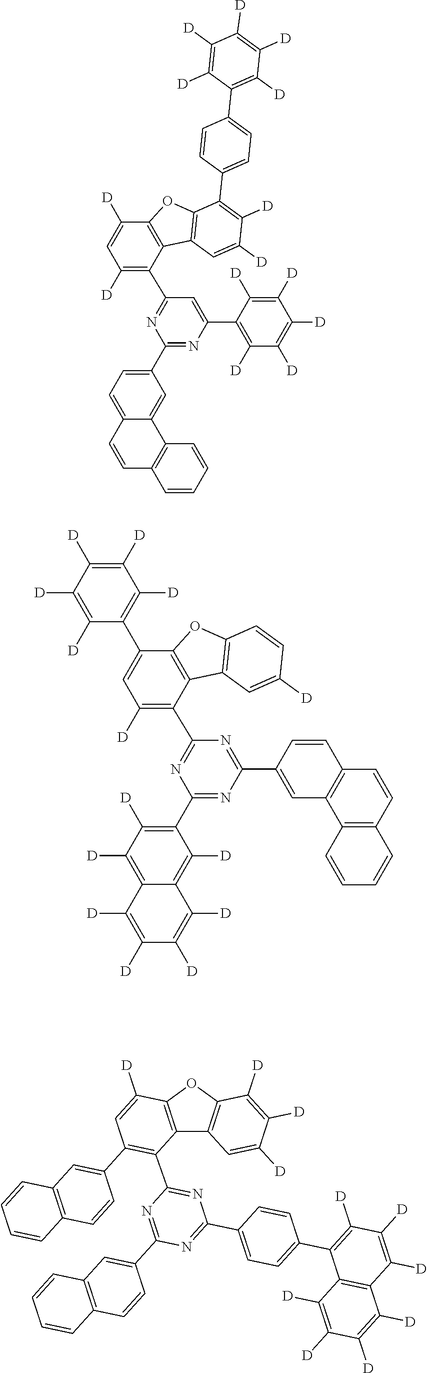 [SMILES: [2H]c1cc2c(oc3c([2H])cc([2H])c(-c4cc(-c5c([2H])c([2H])c([2H])c([2H])c5[2H])nc(-c5ccc6ccc7ccccc7c6c5)n4)c32)c(-c2ccc(-c3c([2H])c([2H])c([2H])c([2H])c3[2H])cc2)c1[2H] ; [2H]c1ccc2oc3c(-c4c([2H])c([2H])c([2H])c([2H])c4[2H])cc([2H])c(-c4nc(-c5ccc6ccc7ccccc7c6c5)nc(-c5c([2H])c([2H])c6c([2H])c([2H])c([2H])c([2H])c6c5[2H])n4)c3c2c1 ; [2H]c1cc2c(oc3c([2H])cc(-c4ccc5ccccc5c4)c(-c4nc(-c5ccc(-c6c([2H])c([2H])c([2H])c7c([2H])c([2H])c([2H])c([2H])c67)cc5)nc(-c5ccc6ccccc6c5)n4)c32)c([2H])c1[2H]]

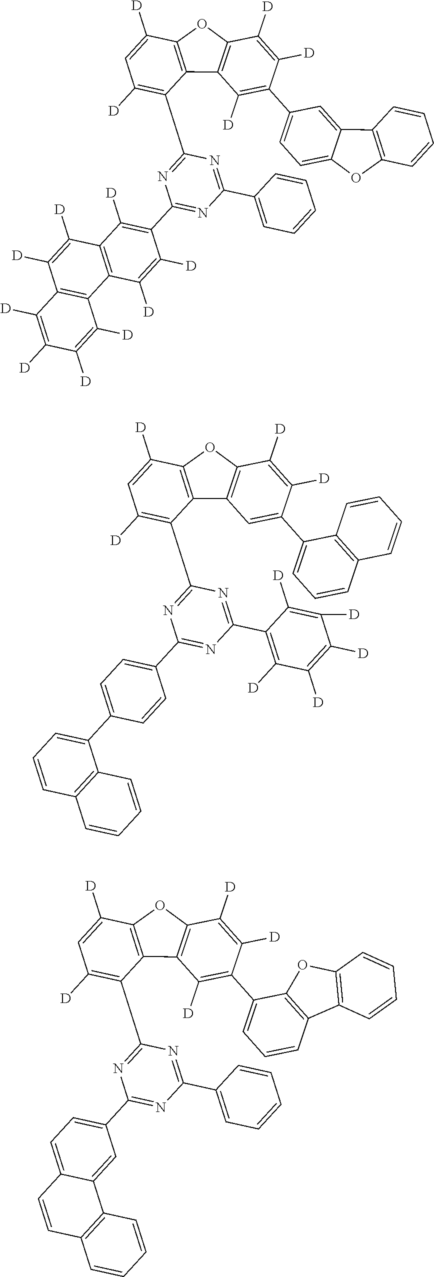 [SMILES: [2H]c1cc([2H])c2oc3c([2H])c([2H])c(-c4ccc5oc6ccccc6c5c4)c([2H])c3c2c1-c1nc(-c2ccccc2)nc(-c2c([2H])c([2H])c3c(c2[2H])c([2H])c([2H])c2c([2H])c([2H])c([2H])c([2H])c23)n1 ; [2H]c1cc([2H])c2oc3c([2H])c([2H])c(-c4cccc5ccccc45)cc3c2c1-c1nc(-c2ccc(-c3cccc4ccccc34)cc2)nc(-c2c([2H])c([2H])c([2H])c([2H])c2[2H])n1 ; [2H]c1cc([2H])c2oc3c([2H])c([2H])c(-c4cccc5c4oc4ccccc45)c([2H])c3c2c1-c1nc(-c2ccccc2)nc(-c2ccc3ccc4ccccc4c3c2)n1]

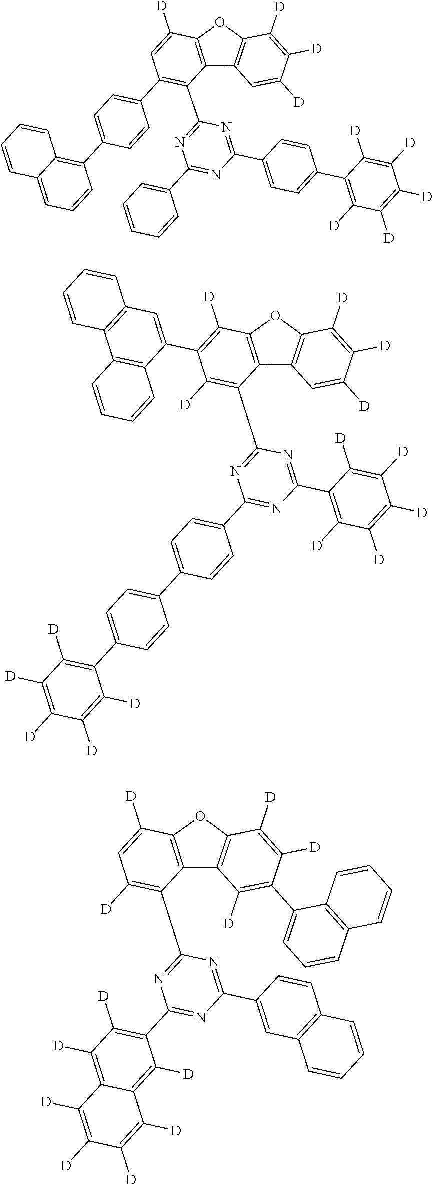 [SMILES: [2H]c1cc2c(oc3c([2H])cc(-c4ccc(-c5cccc6ccccc56)cc4)c(-c4nc(-c5ccccc5)nc(-c5ccc(-c6c([2H])c([2H])c([2H])c([2H])c6[2H])cc5)n4)c32)c([2H])c1[2H] ; [2H]c1cc2c(oc3c([2H])c(-c4cc5ccccc5c5ccccc45)c([2H])c(-c4nc(-c5ccc(-c6ccc(-c7c([2H])c([2H])c([2H])c([2H])c7[2H])cc6)cc5)nc(-c5c([2H])c([2H])c([2H])c([2H])c5[2H])n4)c32)c([2H])c1[2H] ; [2H]c1cc([2H])c2oc3c([2H])c([2H])c(-c4cccc5ccccc45)c([2H])c3c2c1-c1nc(-c2ccc3ccccc3c2)nc(-c2c([2H])c([2H])c3c([2H])c([2H])c([2H])c([2H])c3c2[2H])n1]

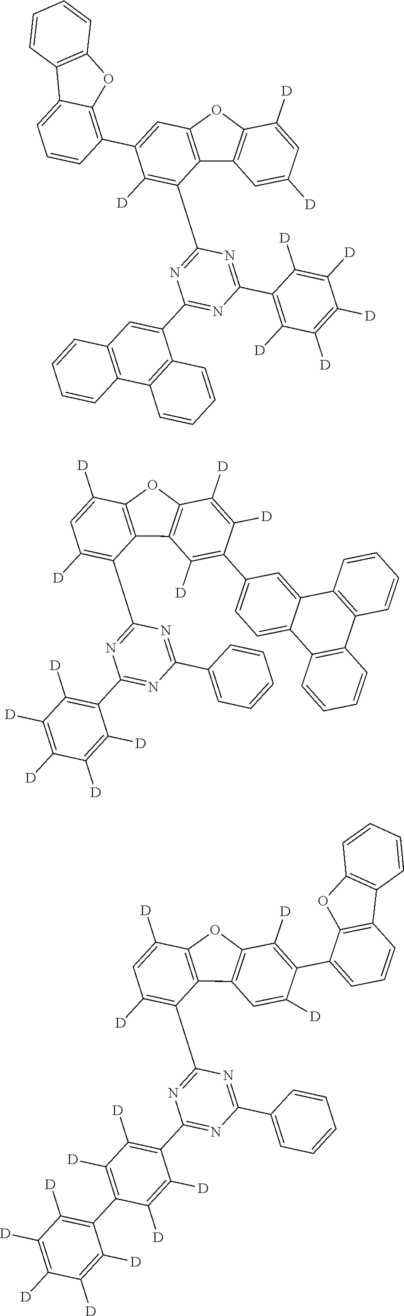 [SMILES: [2H]c1cc([2H])c2oc3cc(-c4cccc5c4oc4ccccc45)c([2H])c(-c4nc(-c5c([2H])c([2H])c([2H])c([2H])c5[2H])nc(-c5cc6ccccc6c6ccccc56)n4)c3c2c1 ; [2H]c1cc([2H])c2oc3c([2H])c([2H])c(-c4ccc5c6ccccc6c6ccccc6c5c4)c([2H])c3c2c1-c1nc(-c2ccccc2)nc(-c2c([2H])c([2H])c([2H])c([2H])c2[2H])n1 ; [2H]c1cc2c(oc3c([2H])cc([2H])c(-c4nc(-c5ccccc5)nc(-c5c([2H])c([2H])c(-c6c([2H])c([2H])c([2H])c([2H])c6[2H])c([2H])c5[2H])n4)c32)c([2H])c1-c1cccc2c1oc1ccccc12]

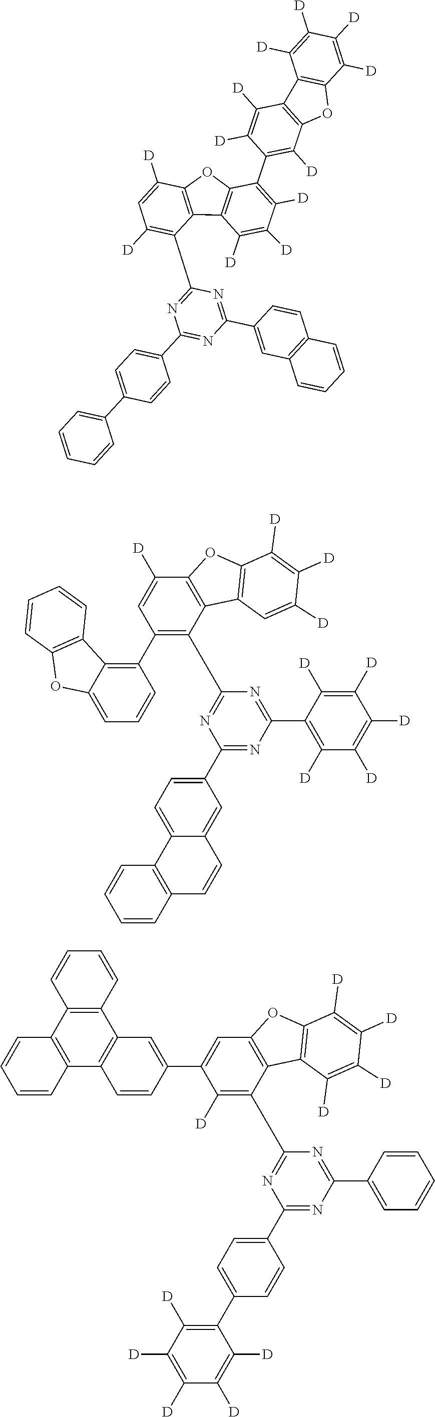

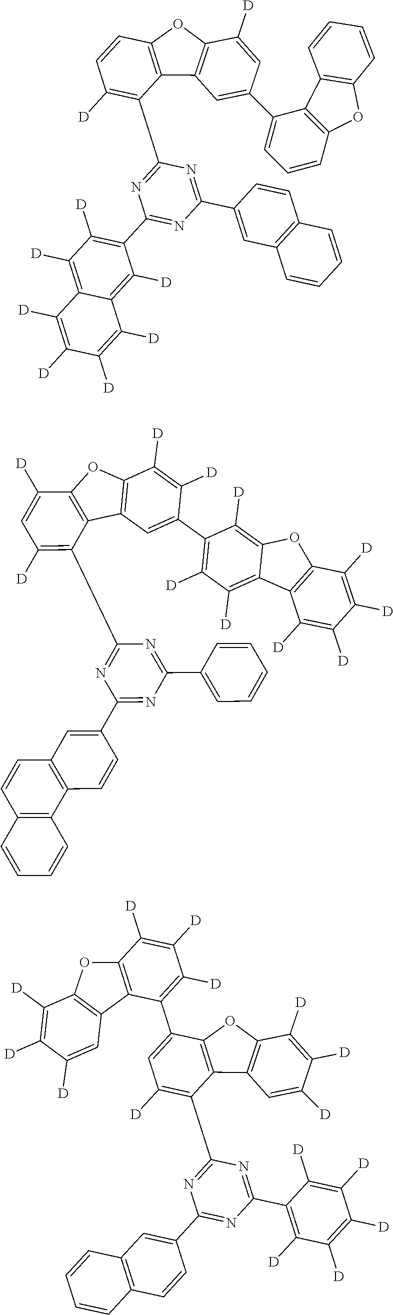 [SMILES: [2H]c1ccc2oc3c([2H])cc(-c4cccc5oc6ccccc6c45)cc3c2c1-c1nc(-c2ccc3ccccc3c2)nc(-c2c([2H])c([2H])c3c([2H])c([2H])c([2H])c([2H])c3c2[2H])n1 ; [2H]c1cc([2H])c2oc3c([2H])c([2H])c(-c4c([2H])c([2H])c5c(oc6c([2H])c([2H])c([2H])c([2H])c65)c4[2H])cc3c2c1-c1nc(-c2ccccc2)nc(-c2ccc3c(ccc4ccccc43)c2)n1 ; [2H]c1cc2c(oc3c(-c4c([2H])c([2H])c([2H])c5oc6c([2H])c([2H])c([2H])cc6c45)cc([2H])c(-c4nc(-c5ccc6ccccc6c5)nc(-c5c([2H])c([2H])c([2H])c([2H])c5[2H])n4)c32)c([2H])c1[2H]]

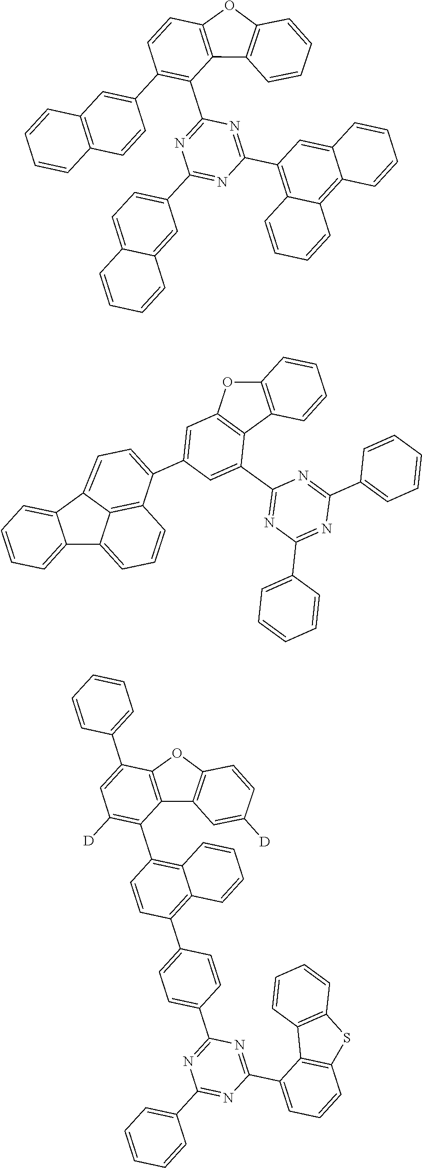 [SMILES: c1ccc2cc(-c3nc(-c4cc5ccccc5c5ccccc45)nc(-c4c(-c5ccc6ccccc6c5)ccc5oc6ccccc6c45)n3)ccc2c1 ; c1ccc(-c2nc(-c3ccccc3)nc(-c3cc(-c4ccc5c6c(cccc46)-c4ccccc4-5)cc4oc5ccccc5c34)n2)cc1 ; [2H]c1ccc2oc3c(-c4ccccc4)cc([2H])c(-c4ccc(-c5ccc(-c6nc(-c7ccccc7)nc(-c7cccc8sc9ccccc9c78)n6)cc5)c5ccccc45)c3c2c1]

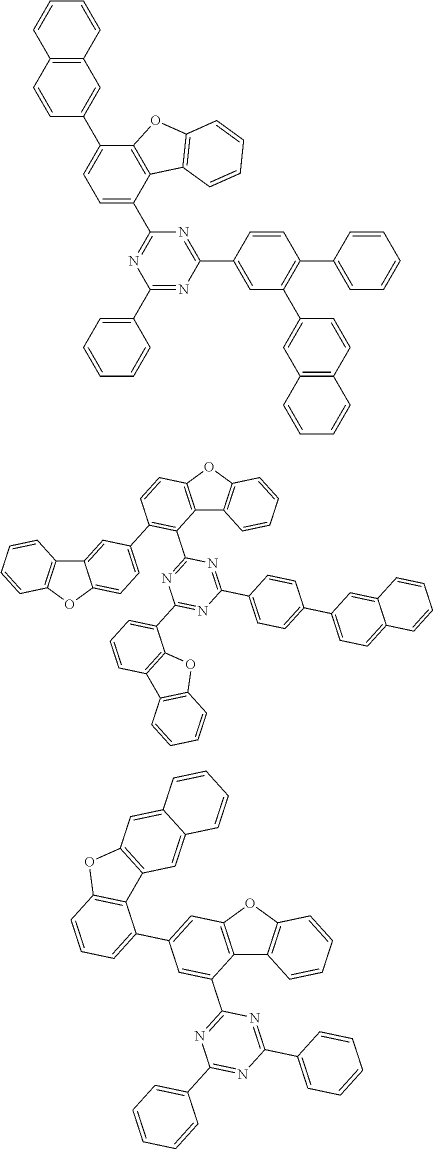

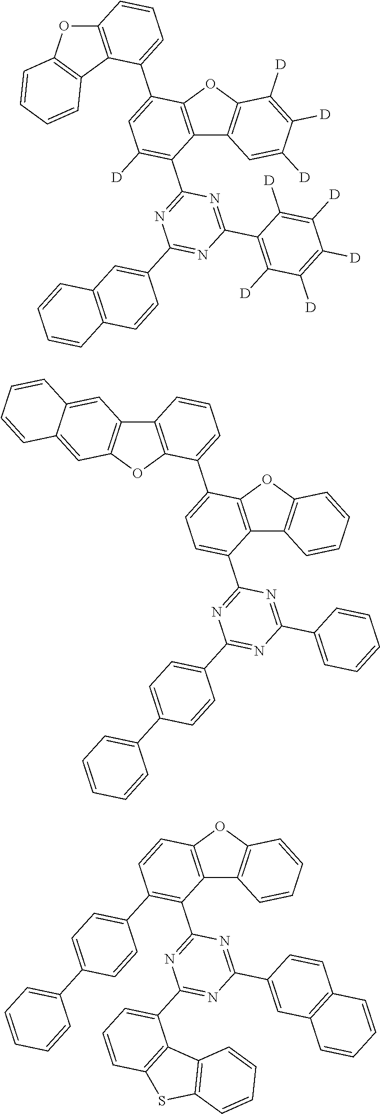

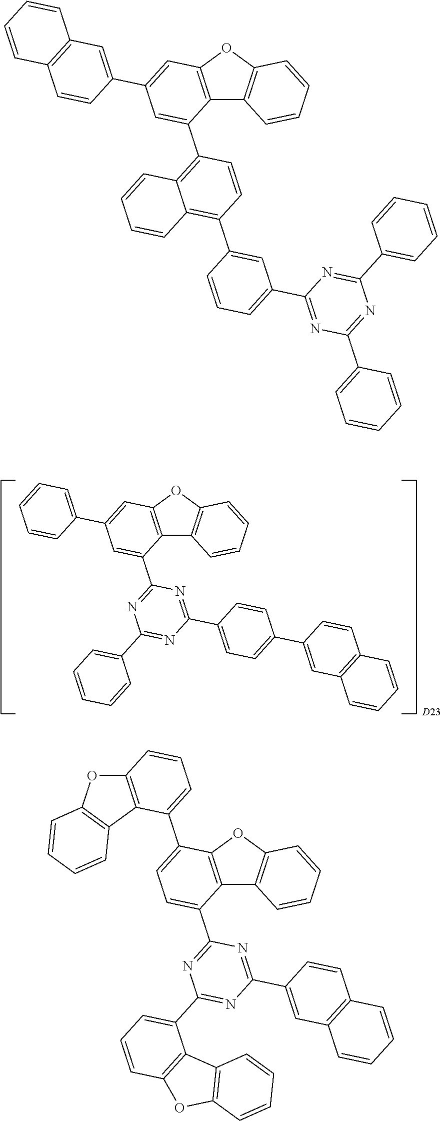

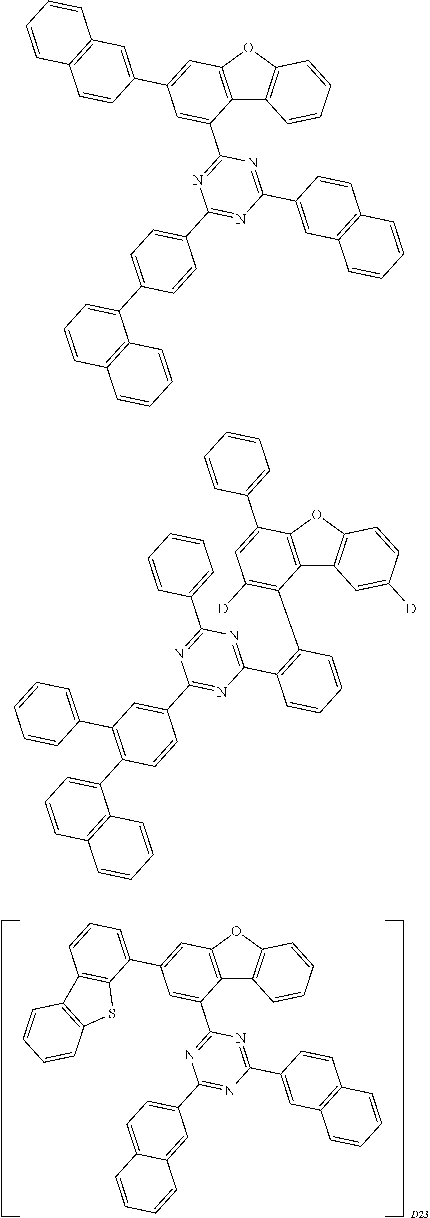

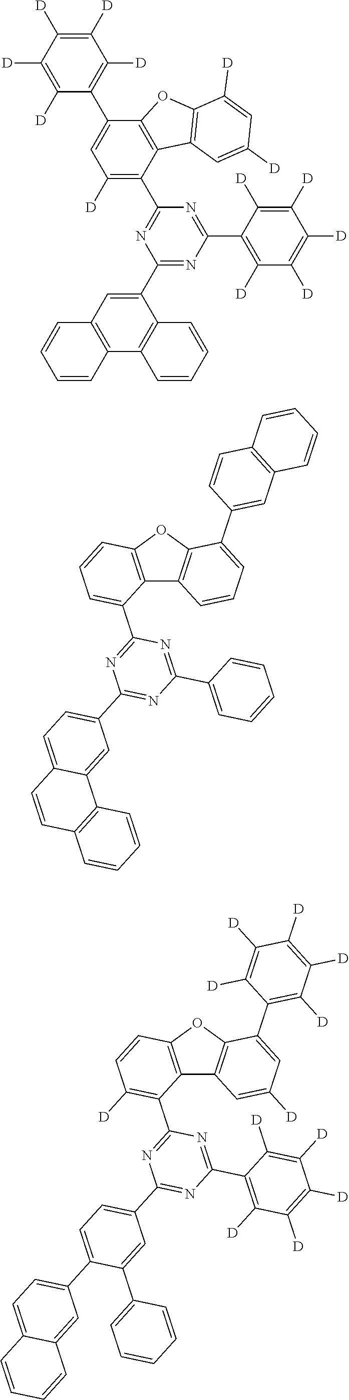 [SMILES: [2H]c1cc([2H])c2oc3c(-c4c([2H])c([2H])c([2H])c([2H])c4[2H])cc([2H])c(-c4nc(-c5c([2H])c([2H])c([2H])c([2H])c5[2H])nc(-c5cc6ccccc6c6ccccc56)n4)c3c2c1 ; c1ccc(-c2nc(-c3ccc4ccc5ccccc5c4c3)nc(-c3cccc4oc5c(-c6ccc7ccccc7c6)cccc5c34)n2)cc1 ; [2H]c1cc(-c2c([2H])c([2H])c([2H])c([2H])c2[2H])c2oc3ccc([2H])c(-c4nc(-c5ccc(-c6ccc7ccccc7c6)c(-c6ccccc6)c5)nc(-c5c([2H])c([2H])c([2H])c([2H])c5[2H])n4)c3c2c1]

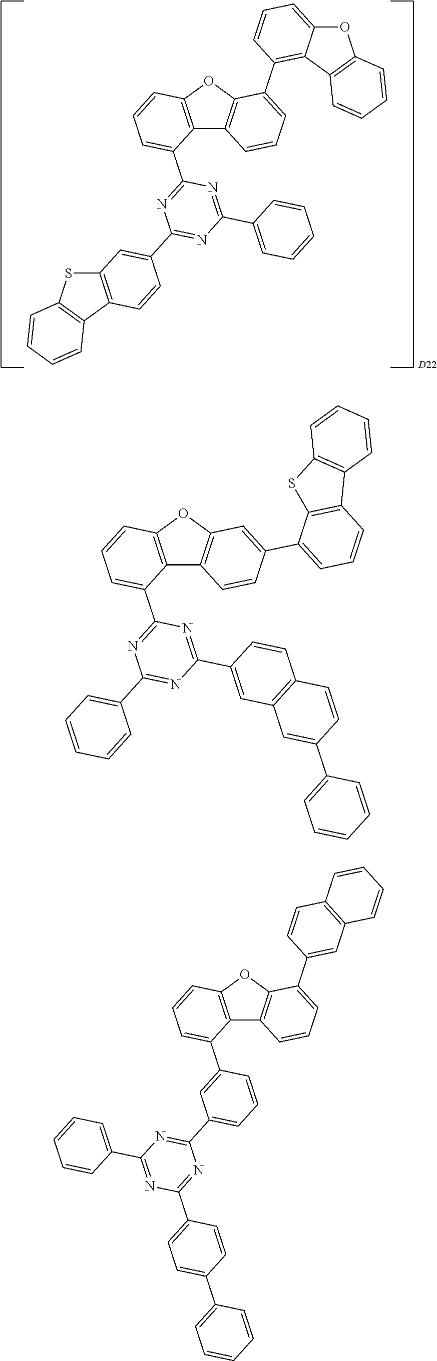

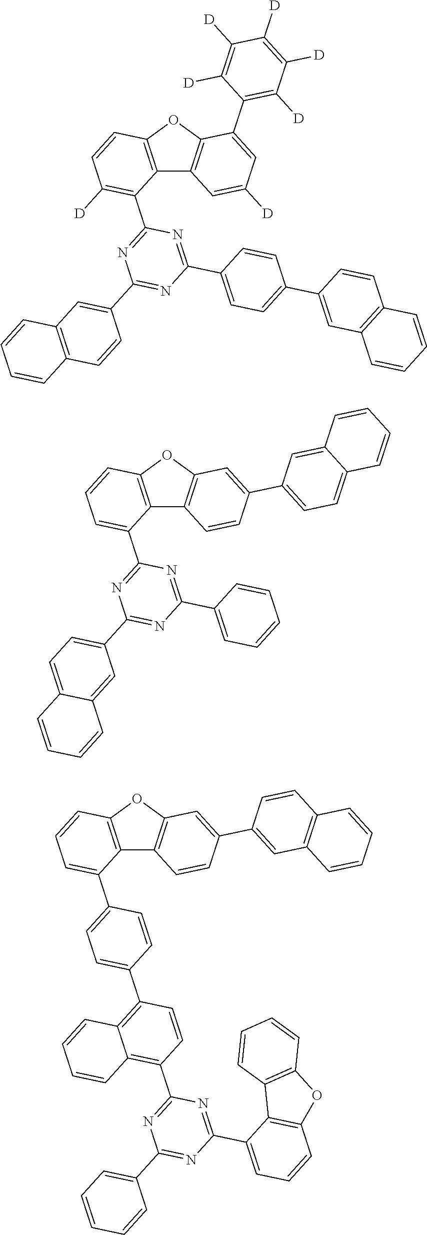

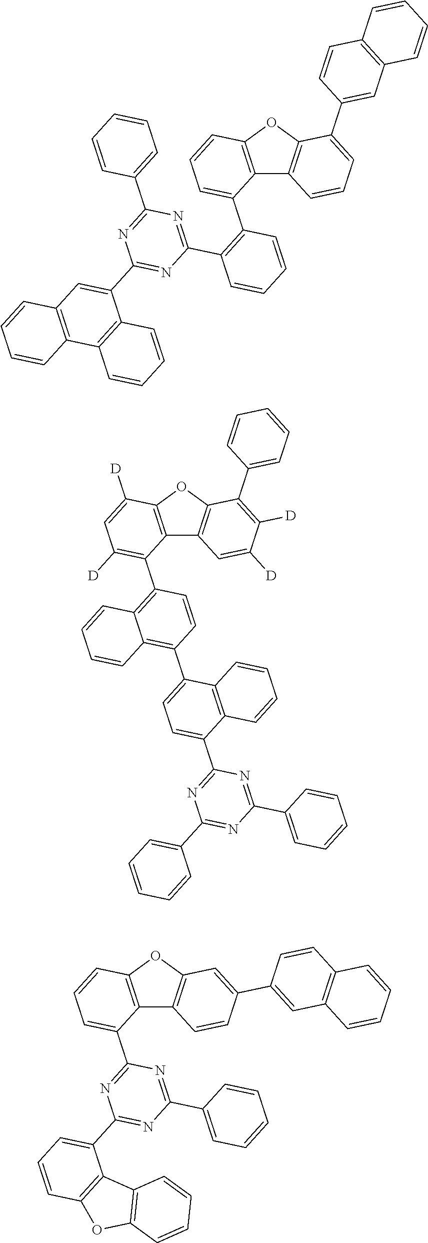 [SMILES: c1ccc(-c2nc(-c3ccccc3-c3cccc4oc5c(-c6ccc7ccccc7c6)cccc5c34)nc(-c3cc4ccccc4c4ccccc34)n2)cc1 ; [2H]c1cc2c(oc3c([2H])cc([2H])c(-c4ccc(-c5ccc(-c6nc(-c7ccccc7)nc(-c7ccccc7)n6)c6ccccc56)c5ccccc45)c32)c(-c2ccccc2)c1[2H] ; c1ccc(-c2nc(-c3cccc4oc5ccccc5c34)nc(-c3cccc4oc5cc(-c6ccc7ccccc7c6)ccc5c34)n2)cc1]

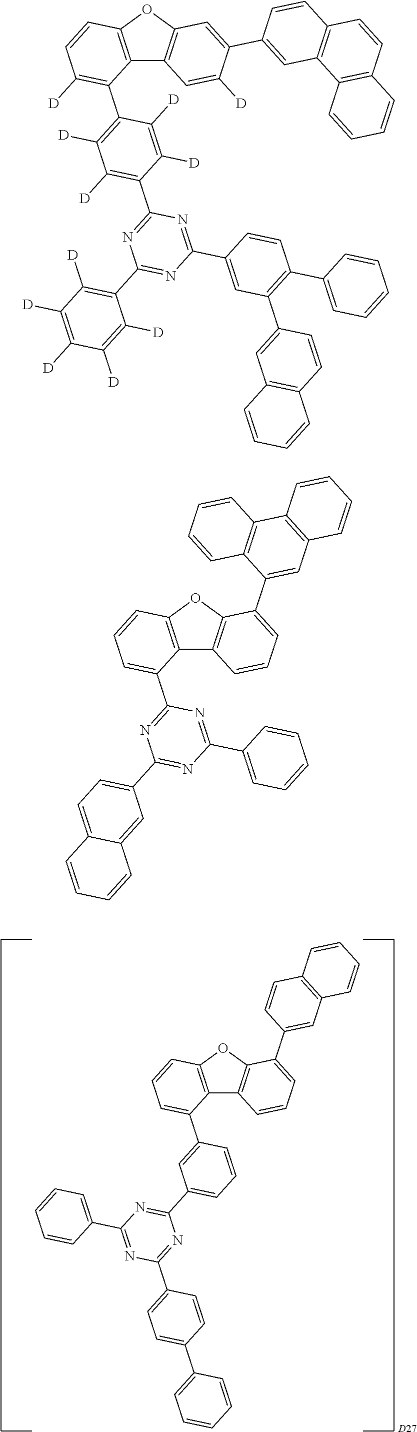

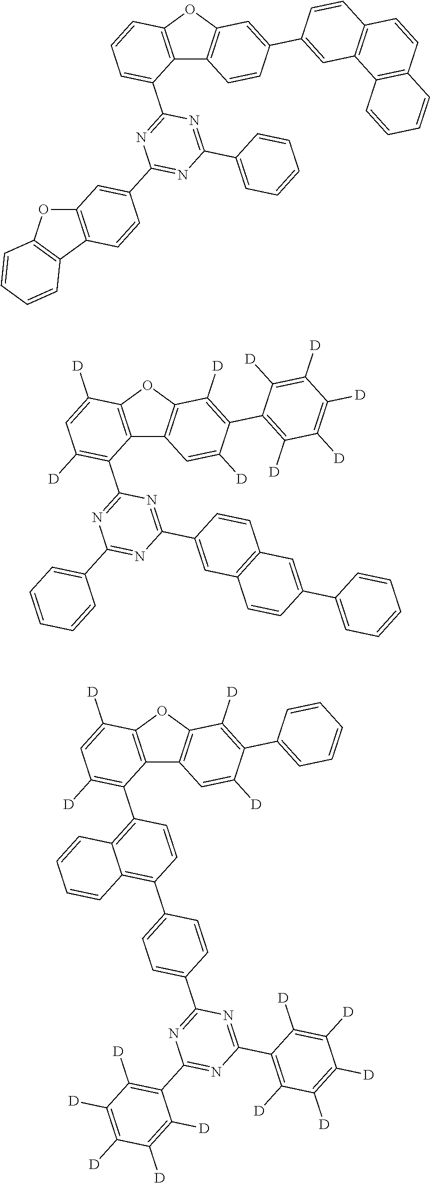 [SMILES: c1ccc(-c2nc(-c3ccc4c(c3)oc3ccccc34)nc(-c3cccc4oc5cc(-c6ccc7ccc8ccccc8c7c6)ccc5c34)n2)cc1 ; [2H]c1cc2c(oc3c([2H])cc([2H])c(-c4nc(-c5ccccc5)nc(-c5ccc6cc(-c7ccccc7)ccc6c5)n4)c32)c([2H])c1-c1c([2H])c([2H])c([2H])c([2H])c1[2H] ; [2H]c1cc2c(oc3c([2H])cc([2H])c(-c4ccc(-c5ccc(-c6nc(-c7c([2H])c([2H])c([2H])c([2H])c7[2H])nc(-c7c([2H])c([2H])c([2H])c([2H])c7[2H])n6)cc5)c5ccccc45)c32)c([2H])c1-c1ccccc1]

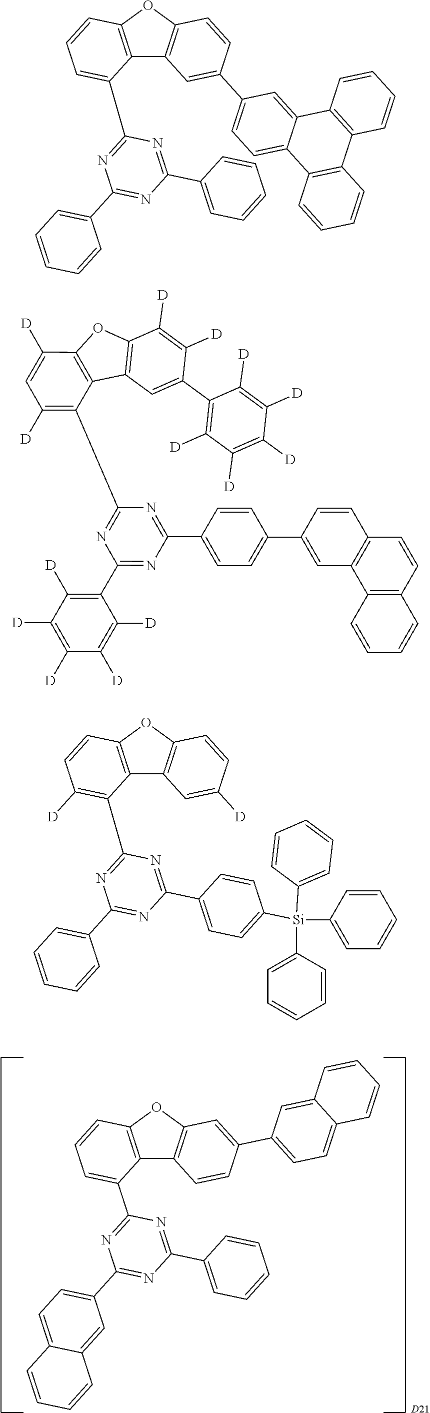

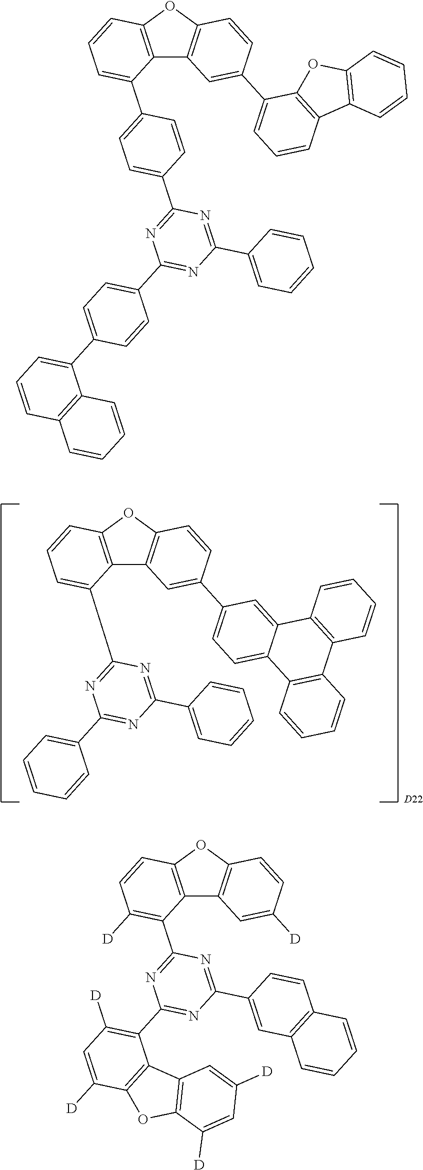

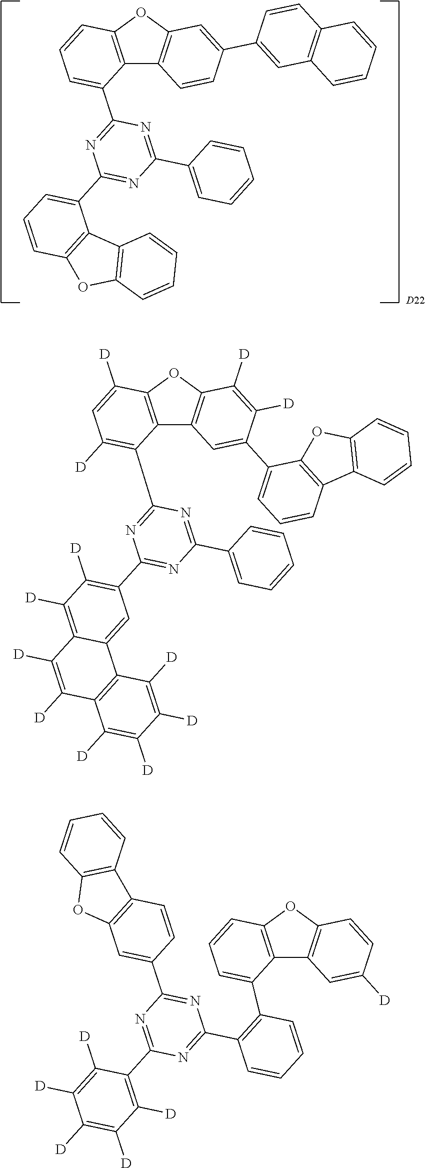

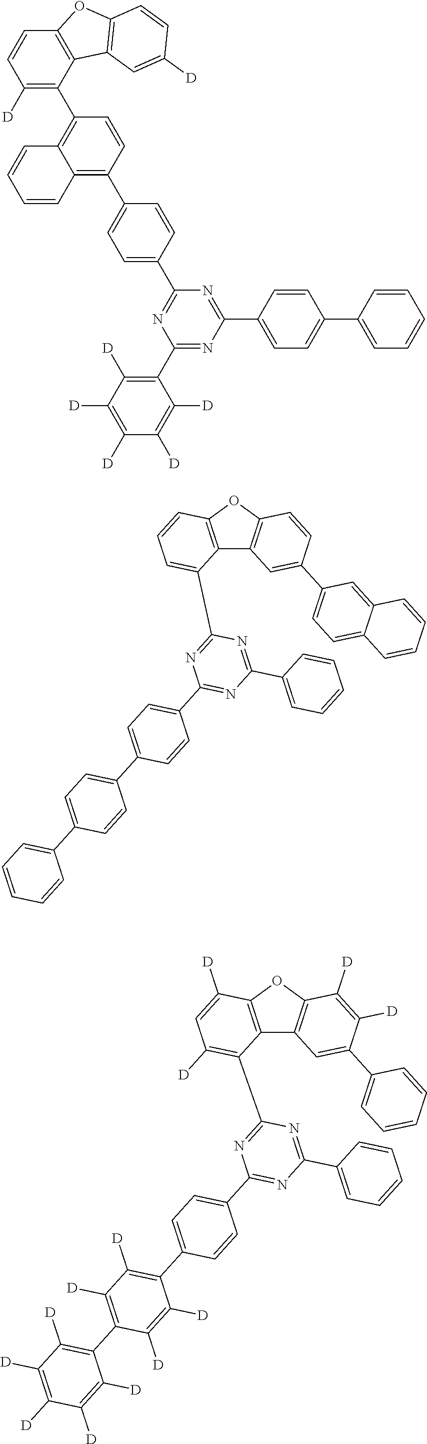 [SMILES: [2H]c1ccc2oc3ccc([2H])c(-c4ccc(-c5ccc(-c6nc(-c7ccc(-c8ccccc8)cc7)nc(-c7c([2H])c([2H])c([2H])c([2H])c7[2H])n6)cc5)c5ccccc45)c3c2c1 ; c1ccc(-c2ccc(-c3ccc(-c4nc(-c5ccccc5)nc(-c5cccc6oc7ccc(-c8ccc9ccccc9c8)cc7c56)n4)cc3)cc2)cc1 ; [2H]c1cc([2H])c2oc3c([2H])c([2H])c(-c4ccccc4)cc3c2c1-c1nc(-c2ccccc2)nc(-c2ccc(-c3c([2H])c([2H])c(-c4c([2H])c([2H])c([2H])c([2H])c4[2H])c([2H])c3[2H])cc2)n1]

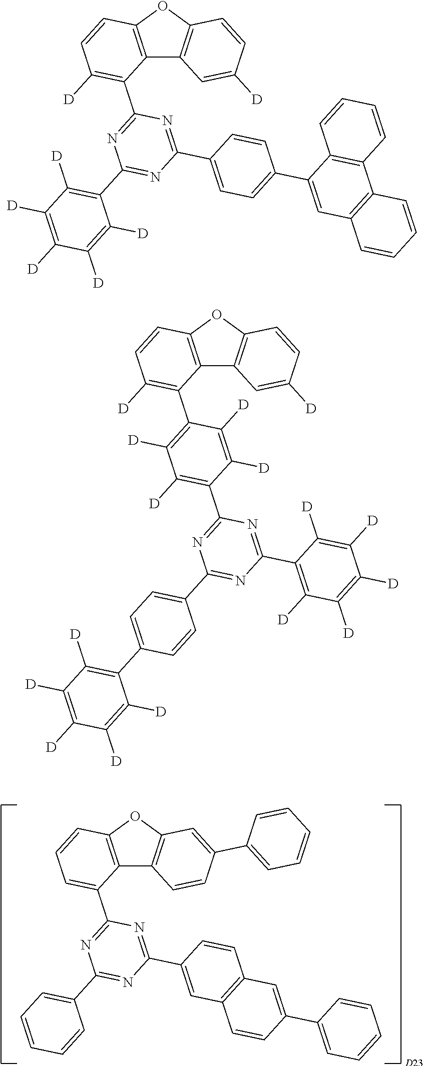

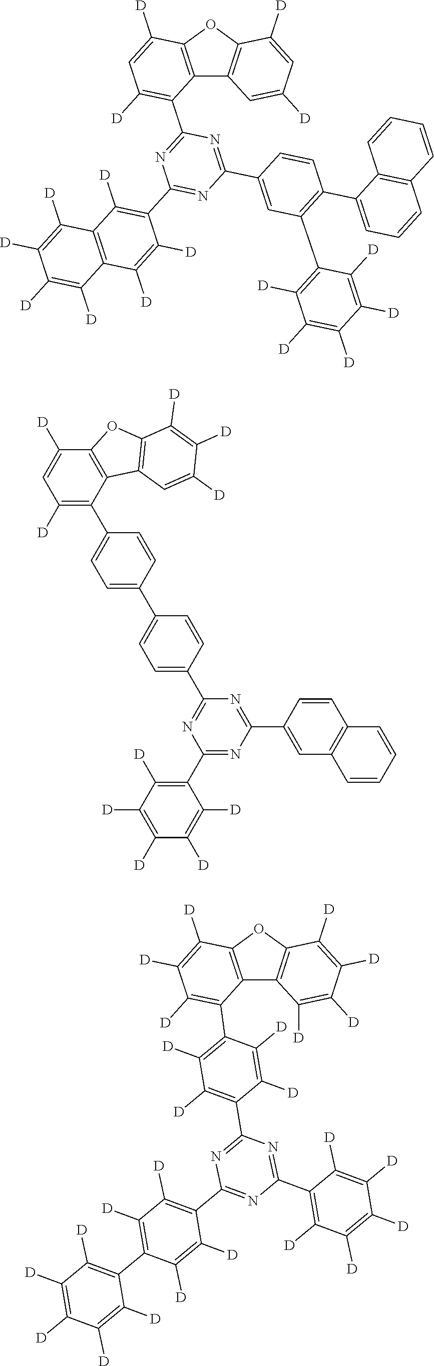

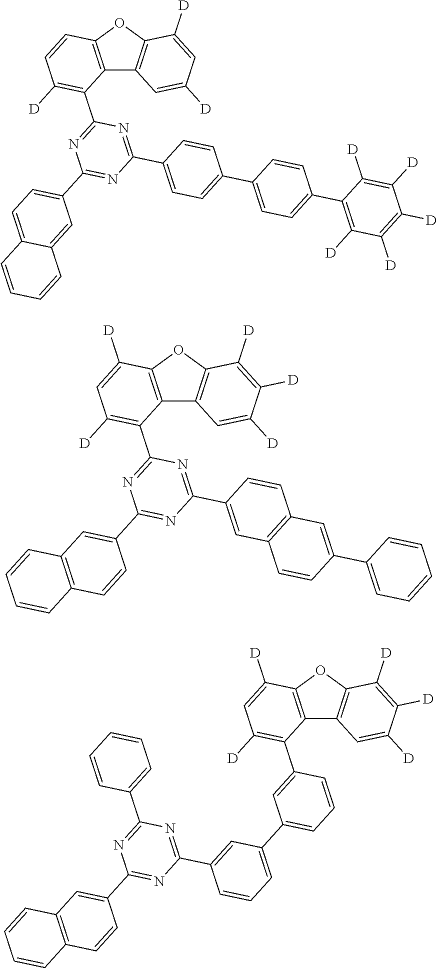 [SMILES: [2H]c1cc([2H])c2oc3ccc([2H])c(-c4nc(-c5ccc(-c6ccc(-c7c([2H])c([2H])c([2H])c([2H])c7[2H])cc6)cc5)nc(-c5ccc6ccccc6c5)n4)c3c2c1 ; [2H]c1cc2c(oc3c([2H])cc([2H])c(-c4nc(-c5ccc6ccccc6c5)nc(-c5ccc6cc(-c7ccccc7)ccc6c5)n4)c32)c([2H])c1[2H] ; [2H]c1cc2c(oc3c([2H])cc([2H])c(-c4cccc(-c5cccc(-c6nc(-c7ccccc7)nc(-c7ccc8ccccc8c7)n6)c5)c4)c32)c([2H])c1[2H]]

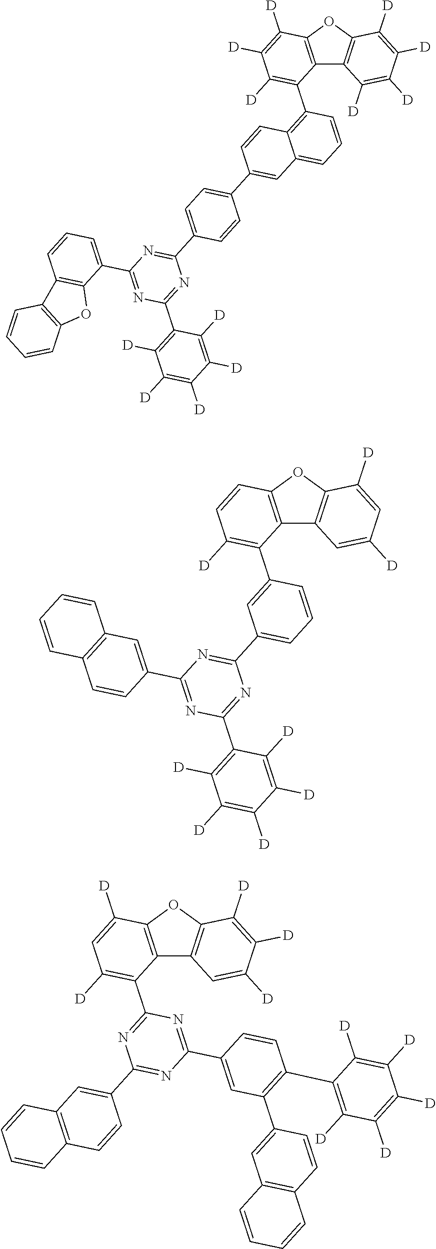

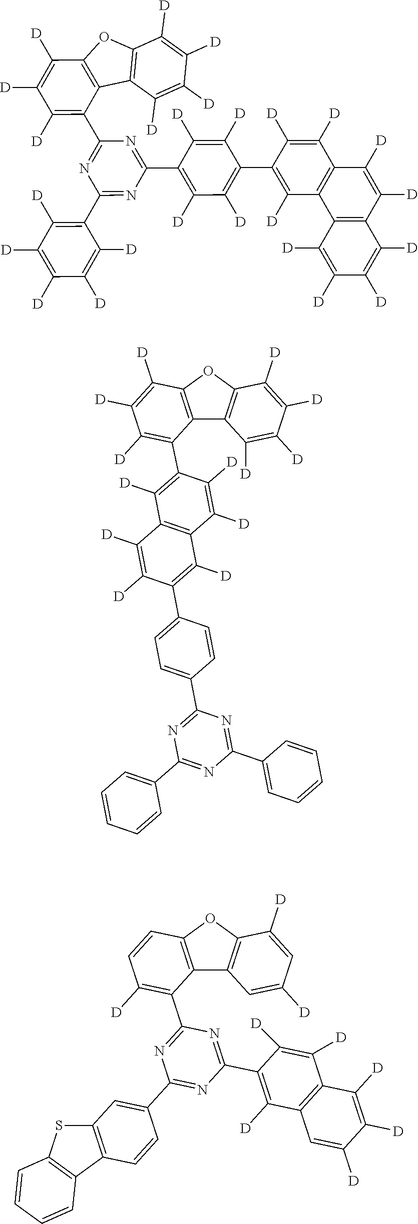 [SMILES: [2H]c1c([2H])c([2H])c(-c2nc(-c3c([2H])c([2H])c(-c4c([2H])c([2H])c5c([2H])c([2H])c6c([2H])c([2H])c([2H])c([2H])c6c5c4[2H])c([2H])c3[2H])nc(-c3c([2H])c([2H])c([2H])c4oc5c([2H])c([2H])c([2H])c([2H])c5c34)n2)c([2H])c1[2H] ; [2H]c1c([2H])c([2H])c2c(oc3c([2H])c([2H])c([2H])c(-c4c([2H])c([2H])c5c([2H])c(-c6ccc(-c7nc(-c8ccccc8)nc(-c8ccccc8)n7)cc6)c([2H])c([2H])c5c4[2H])c32)c1[2H] ; [2H]c1cc([2H])c2oc3ccc([2H])c(-c4nc(-c5ccc6c(c5)sc5ccccc56)nc(-c5c([2H])c([2H])c6c([2H])c([2H])c([2H])cc6c5[2H])n4)c3c2c1]

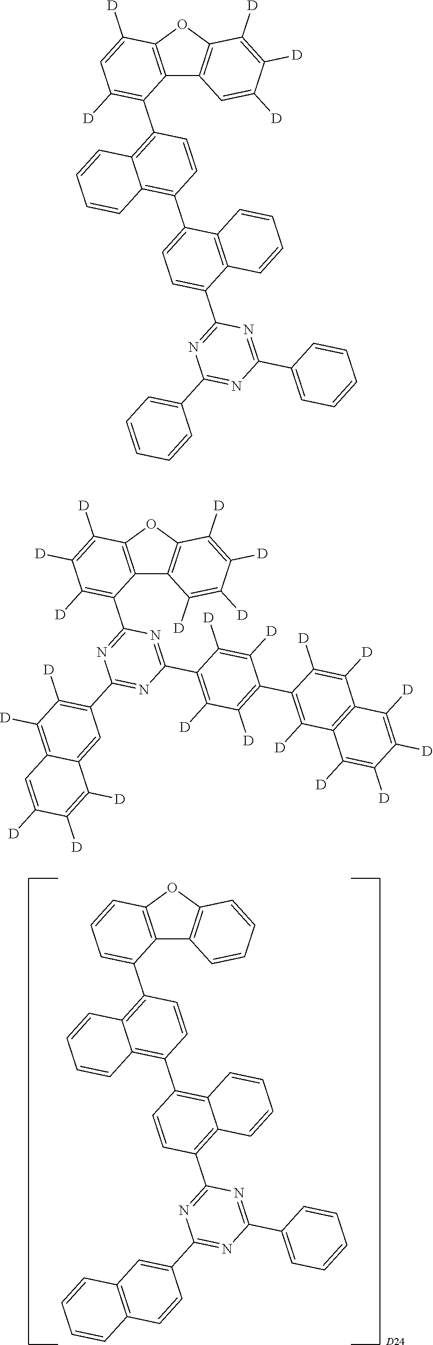

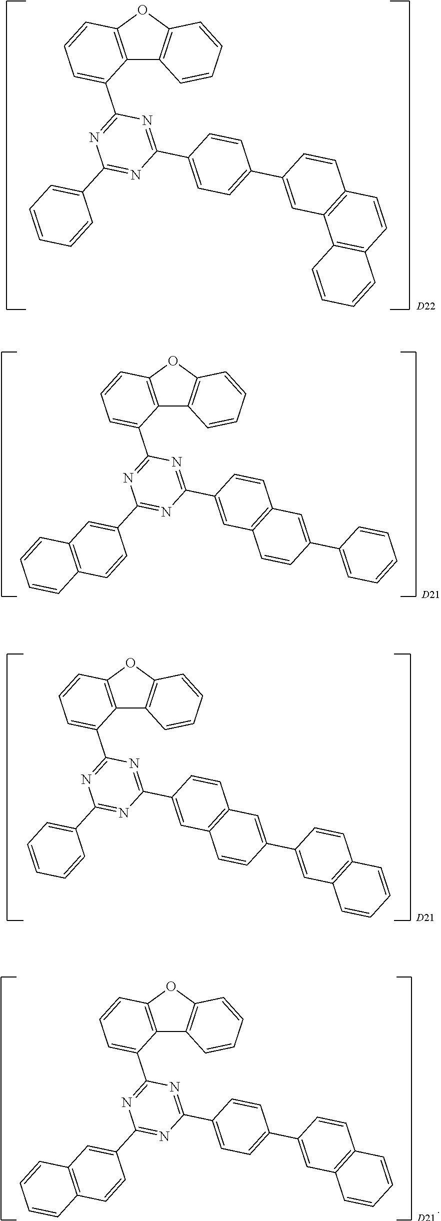

Among the above listed compounds, the compound represented by ‘[structural formula] Dₙ’ is a compound of which the corresponding ‘structural formula’ is substituted with n deuteriums.

The compound represented by Chemical Formula 1, in which a is 1, and R₁ is a substituted or unsubstituted C₆₋₆₀ aryl; or a substituted or unsubstituted C₂₋₆₀ heteroaryl containing one or more selected from the group consisting of N, O and S, can be prepared by a preparation method as shown in the following Reaction Scheme 1 as an example, and other remaining compounds can be prepared in a similar manner.

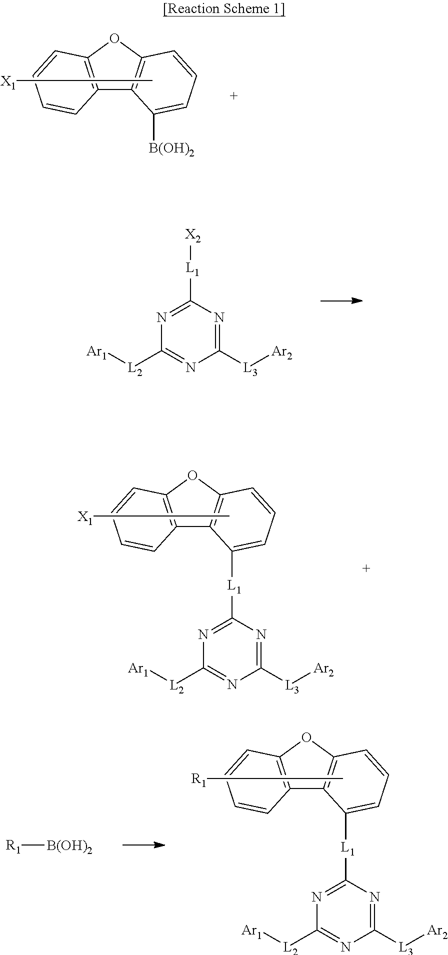

In Reaction Scheme 1, R₁, Ar₁, Ar₂ and L₁ to L₃ are the same as defined in Chemical Formula 1, and X₁ and X₂ are each independently halogen, and preferably X₁ and X₂ are each independently chloro or bromo.

Reaction Scheme 1 is a Suzuki coupling reaction, which is preferably carried out in the presence of a palladium catalyst and a base, and a reactive group for the Suzuki coupling reaction can be modified as known in the art. The preparation method can be further embodied in Preparation Examples described hereinafter.

Preferably, the compound represented by Chemical Formula 2 may be represented by one of the following Chemical Formulas 2-1 to 2-4:

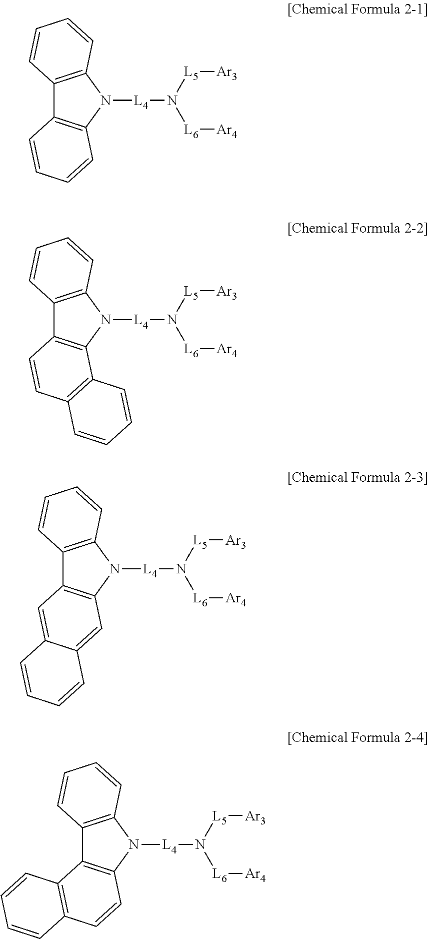

in Chemical Formulas 2-1 to 2-4,
Ar₃, Ar₄ and L₄ to L₆ are the same as defined in Chemical Formula 2.

Preferably, Ar₃ and Ar₄ are each independently a substituted or unsubstituted C₆₋₂₀ aryl, or a substituted or unsubstituted C₂₋₂₀ heteroaryl containing one or more selected from the group consisting of N, O and S.

More preferably, Ar₃ and Ar₄ may be each independently phenyl, biphenylyl, terphenylyl, quaterphenylyl, triphenylmethyl phenyl, triphenylsilyl phenyl, naphthyl, phenanthrenyl, triphenylenyl, fluoranthenyl, chrysenyl, benzo[c]phenanthrenyl, carbazolyl, phenyl carbazolyl, dimethylfluorenyl, dibenzofuranyl, or dibenzothiophenyl.

Most preferably, Ar₃ and Ar₄ may be each independently one selected from the following:

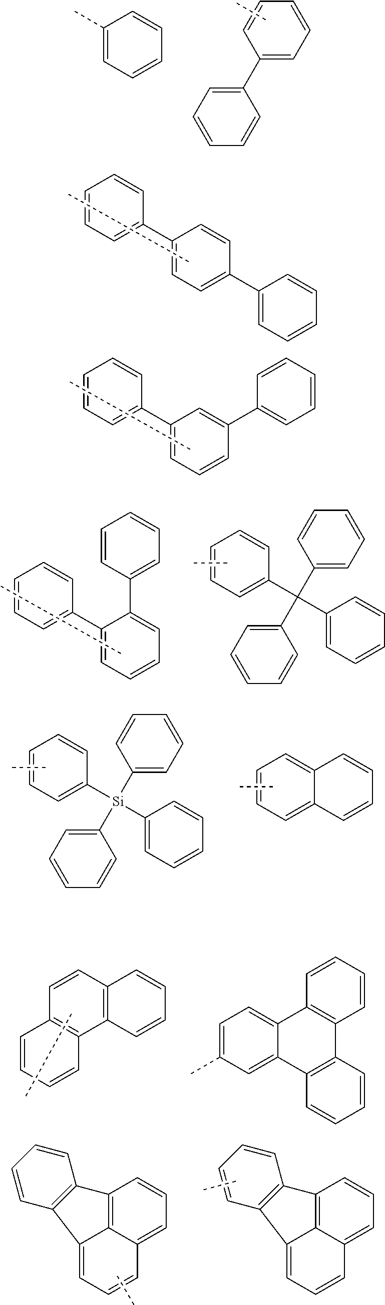

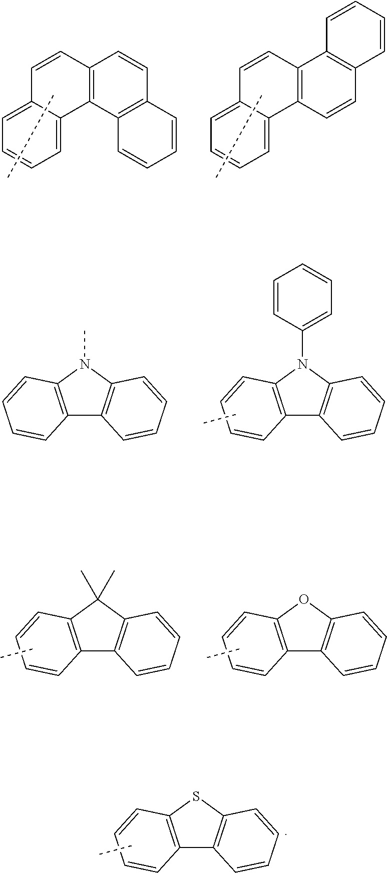

Preferably, L₄ may be a substituted or unsubstituted C₆₋₆₀ arylene.

More preferably, L₄ is phenylene, biphenyldiyl, terphenyldiyl, quarterphenyldiyl, naphthalenediyl, phenylnaphthalenediyl, or phenylnaphthalenediyl substituted with one phenyl.

Most preferably, L₄ may be one selected from the following:

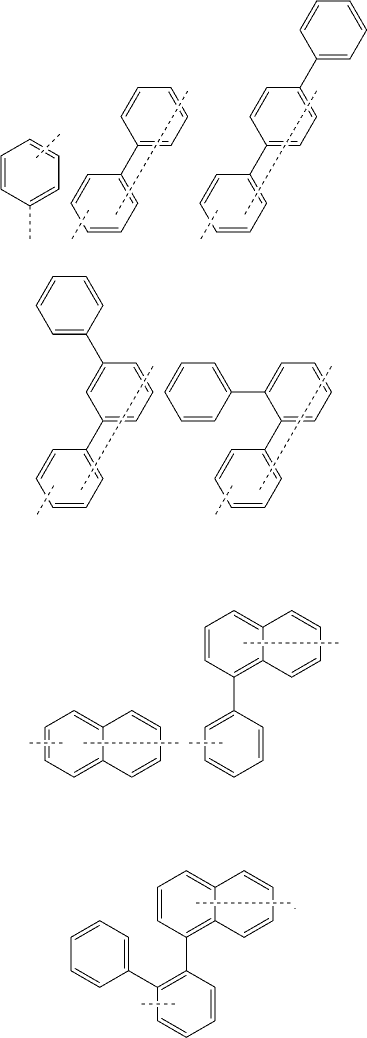

Preferably, L₅ and L₆ may be each independently a single bond, a substituted or unsubstituted C₆₋₂₀ arylene, or a substituted or unsubstituted C₂₋₂₀ heteroarylene containing one or more selected from the group consisting of N, O and S.

More preferably, L₅ and L₆ may be each independently a single bond, phenylene, biphenyldiyl, naphthalenediyl, phenylnaphthalenediyl, or carbazolediyl.

Most preferably, L₅ and L₆ may be each independently a single bond, or one selected from the following:

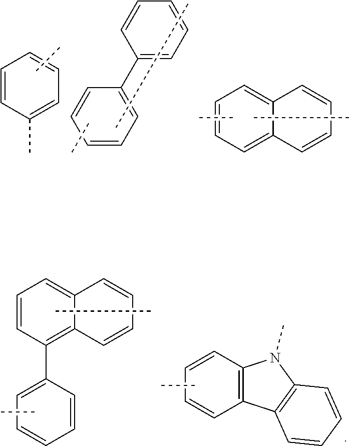

Representative examples of the compound represented by Chemical Formula 2 are as follows:

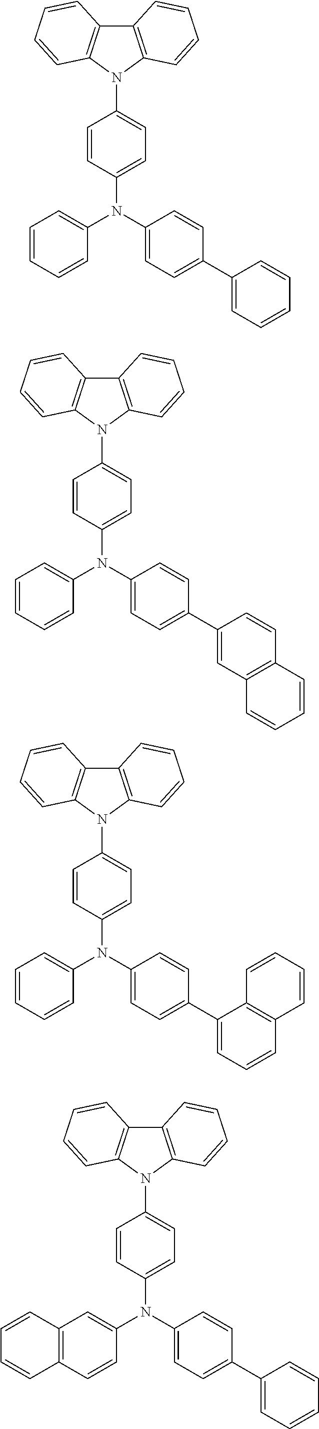 [SMILES: c1ccc(-c2ccc(N(c3ccccc3)c3ccc(-n4c5ccccc5c5ccccc54)cc3)cc2)cc1 ; c1ccc(N(c2ccc(-c3ccc4ccccc4c3)cc2)c2ccc(-n3c4ccccc4c4ccccc43)cc2)cc1 ; c1ccc(N(c2ccc(-c3cccc4ccccc34)cc2)c2ccc(-n3c4ccccc4c4ccccc43)cc2)cc1 ; c1ccc(-c2ccc(N(c3ccc(-n4c5ccccc5c5ccccc54)cc3)c3ccc4ccccc4c3)cc2)cc1]

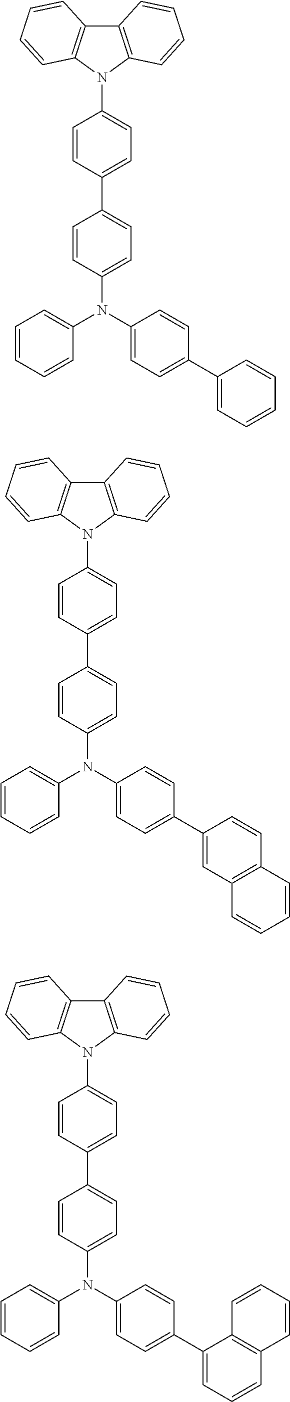 [SMILES: c1ccc(-c2ccc(N(c3ccccc3)c3ccc(-c4ccc(-n5c6ccccc6c6ccccc65)cc4)cc3)cc2)cc1 ; c1ccc(N(c2ccc(-c3ccc(-n4c5ccccc5c5ccccc54)cc3)cc2)c2ccc(-c3ccc4ccccc4c3)cc2)cc1 ; c1ccc(N(c2ccc(-c3ccc(-n4c5ccccc5c5ccccc54)cc3)cc2)c2ccc(-c3cccc4ccccc34)cc2)cc1]

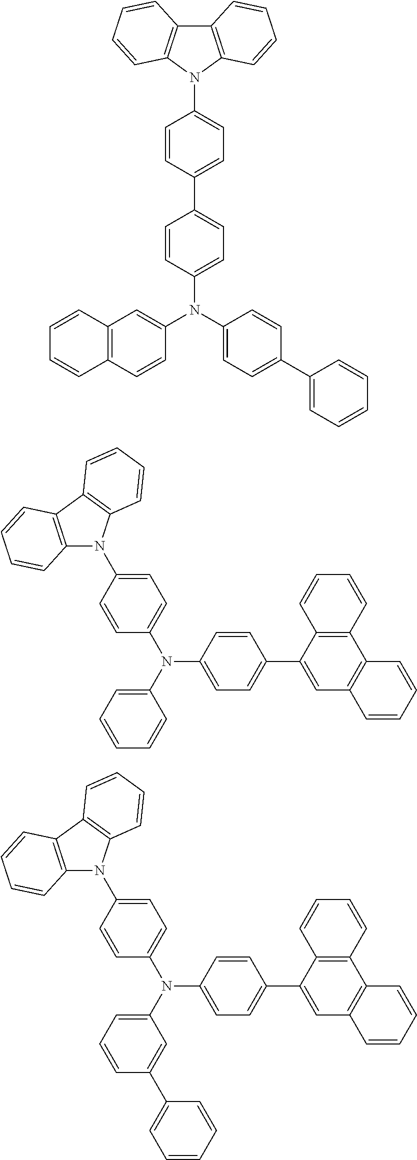

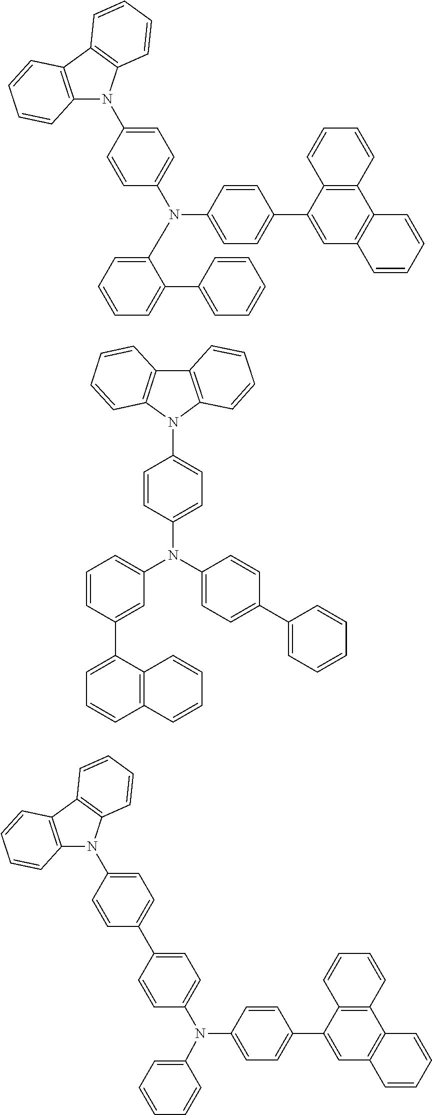

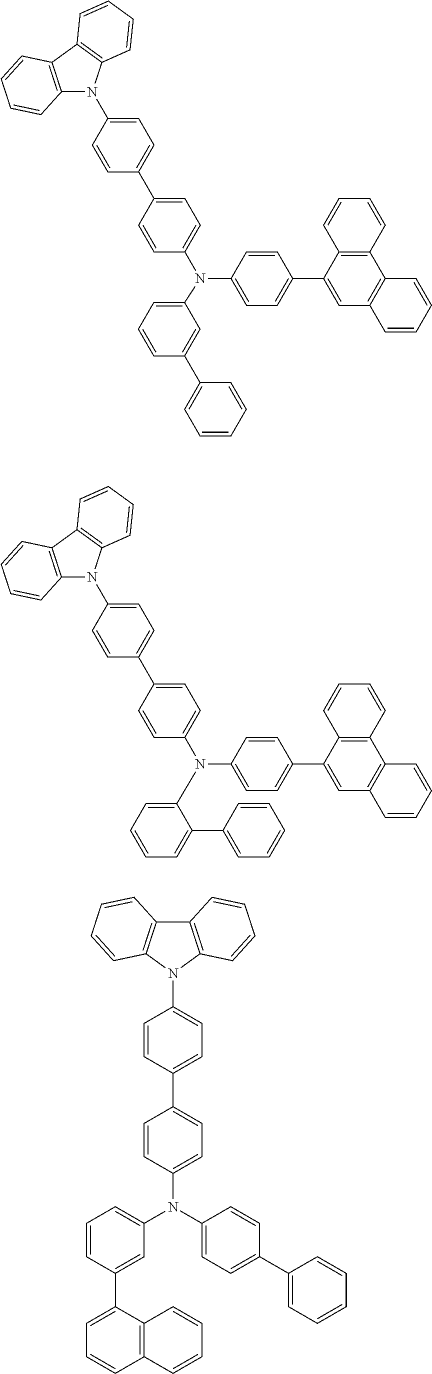

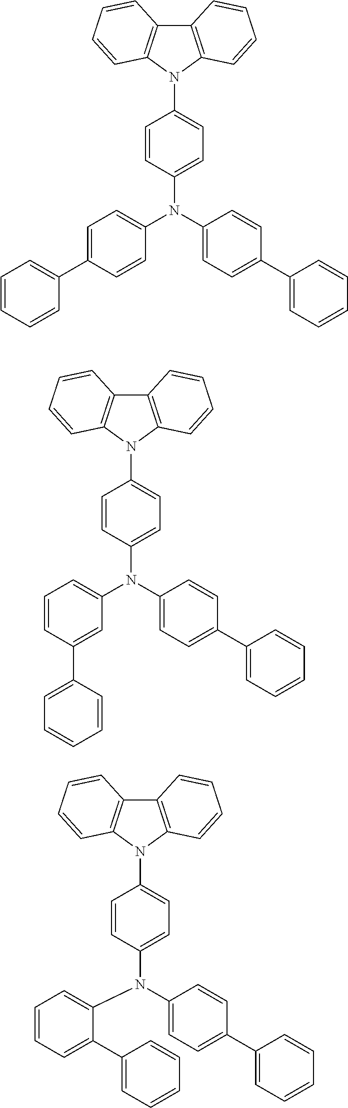

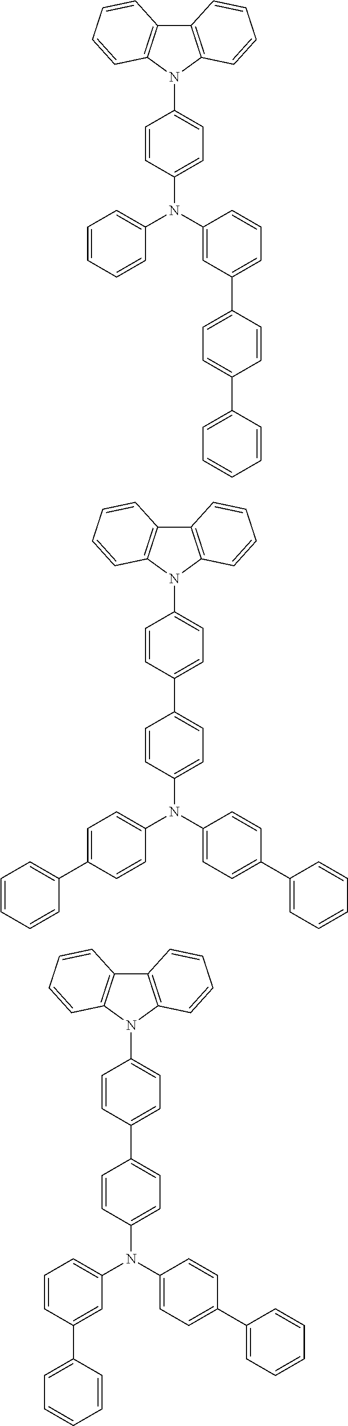

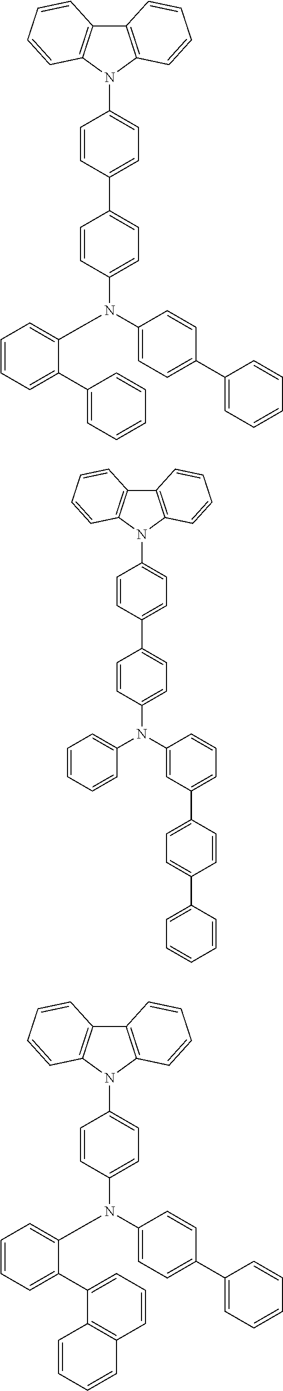

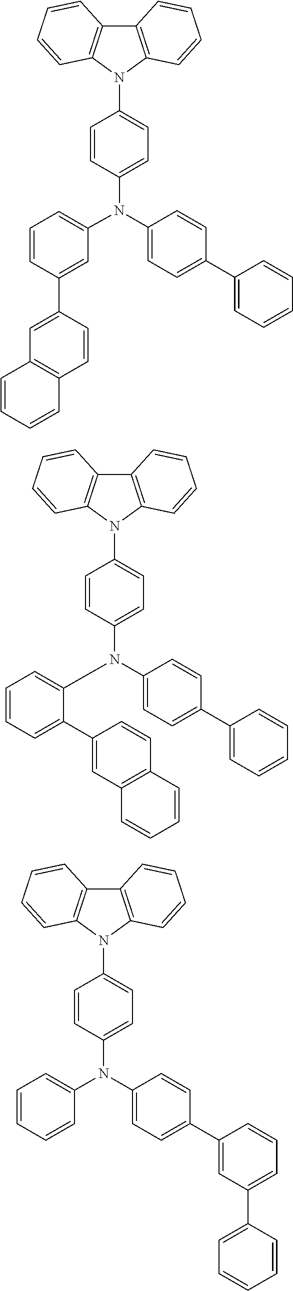

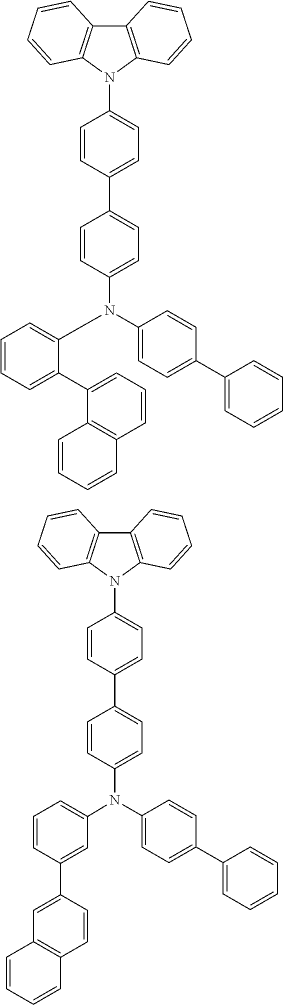

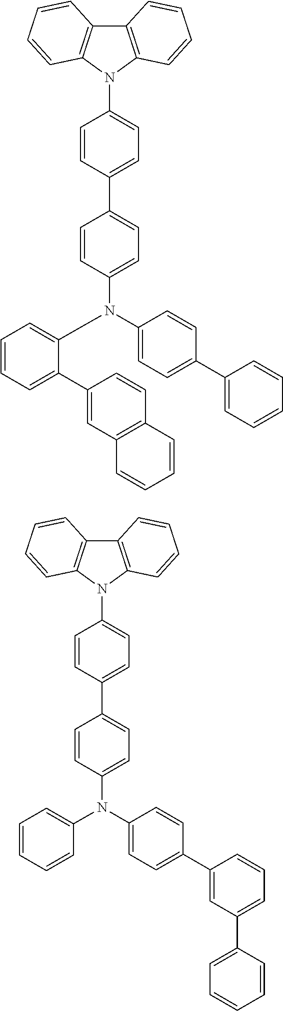

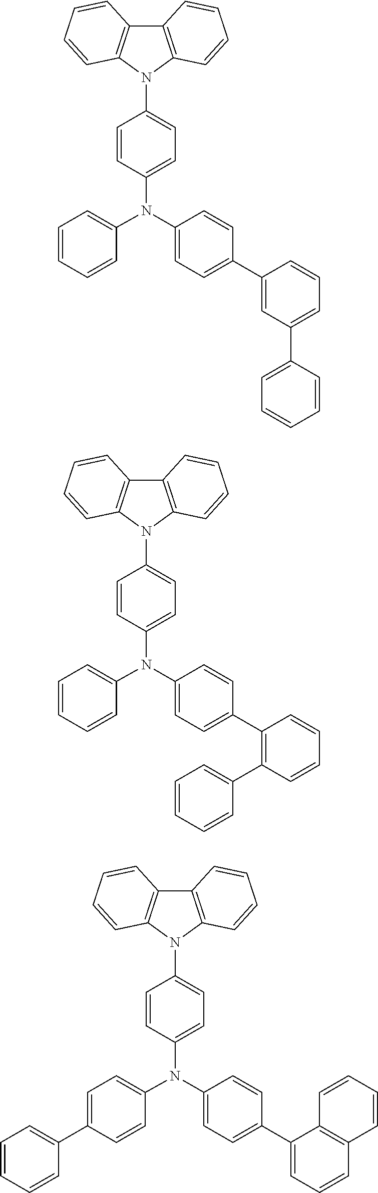

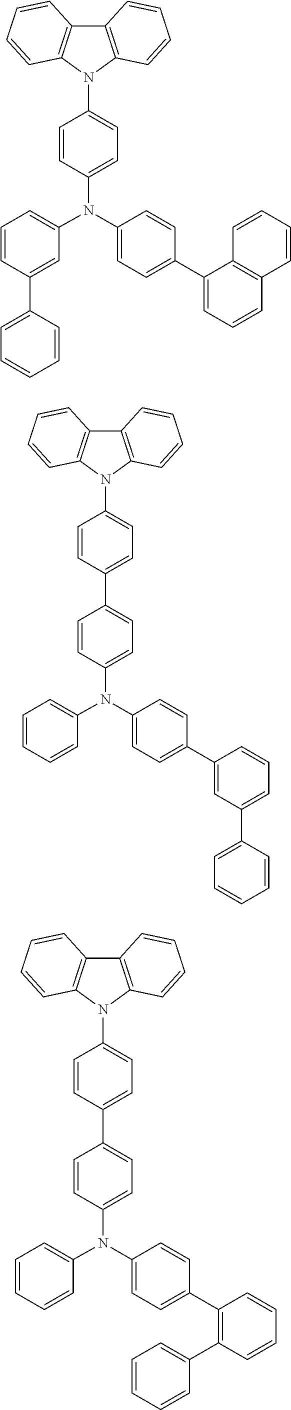

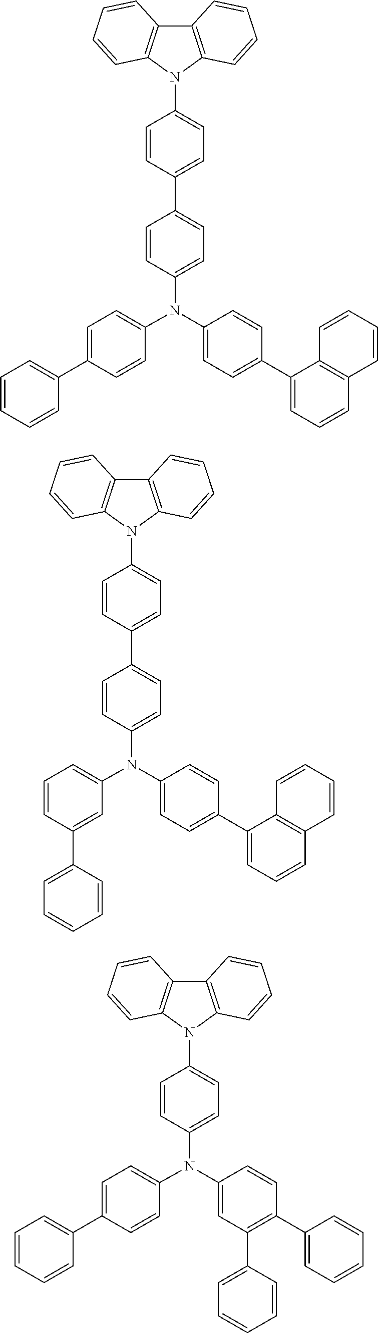

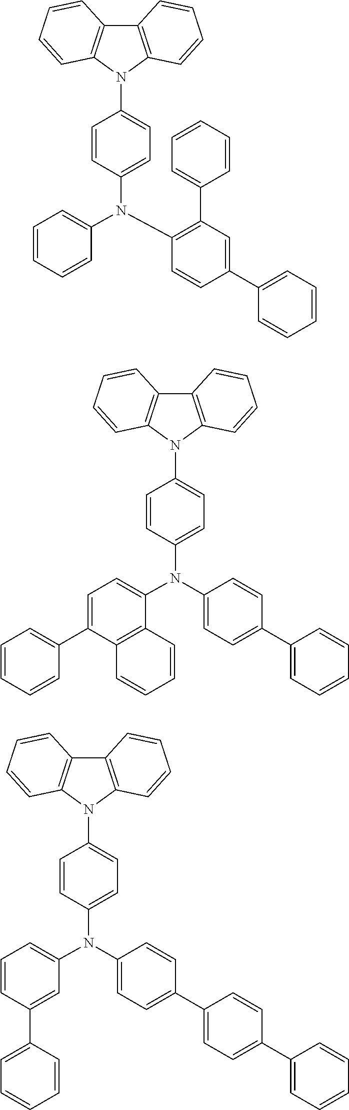

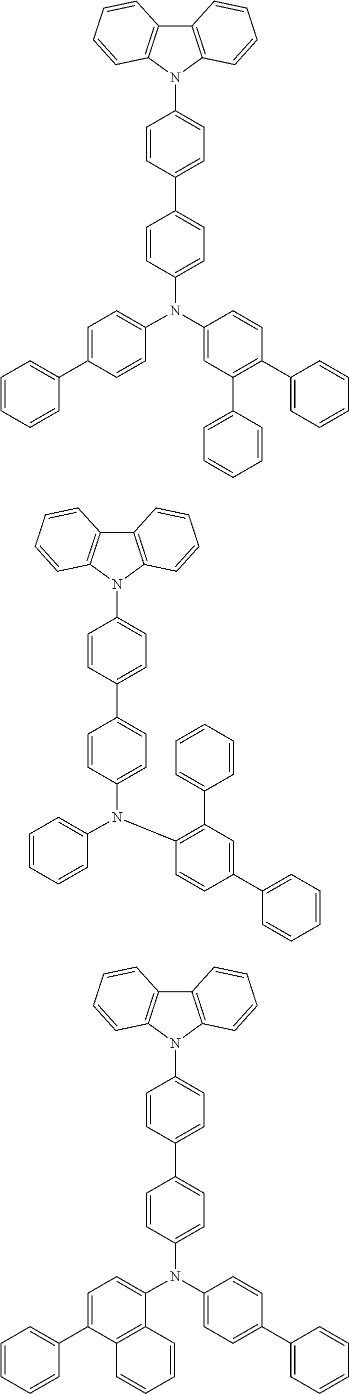 [SMILES: c1ccc(-c2ccc(N(c3ccc(-c4ccc(-n5c6ccccc6c6ccccc65)cc4)cc3)c3ccc(-c4ccccc4)c(-c4ccccc4)c3)cc2)cc1 ; c1ccc(-c2ccc(N(c3ccccc3)c3ccc(-c4ccc(-n5c6ccccc6c6ccccc65)cc4)cc3)c(-c3ccccc3)c2)cc1 ; c1ccc(-c2ccc(N(c3ccc(-c4ccc(-n5c6ccccc6c6ccccc65)cc4)cc3)c3ccc(-c4ccccc4)c4ccccc34)cc2)cc1]

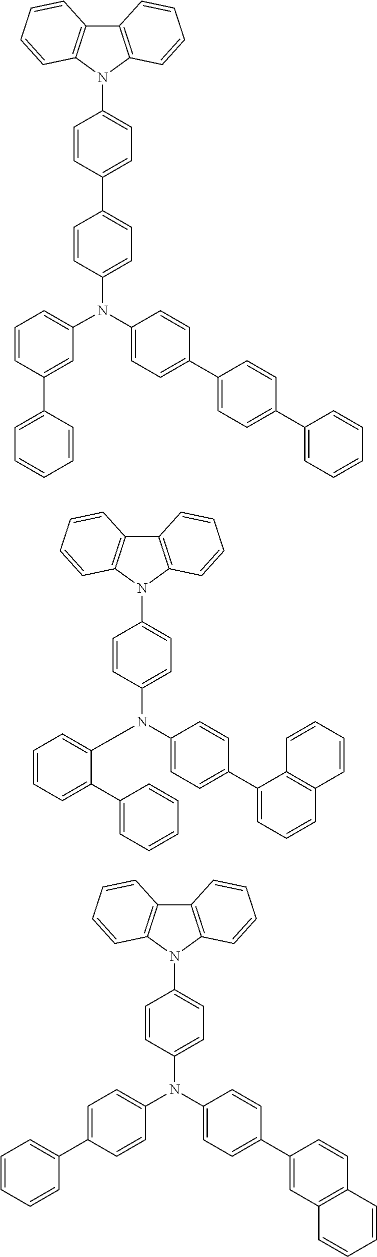

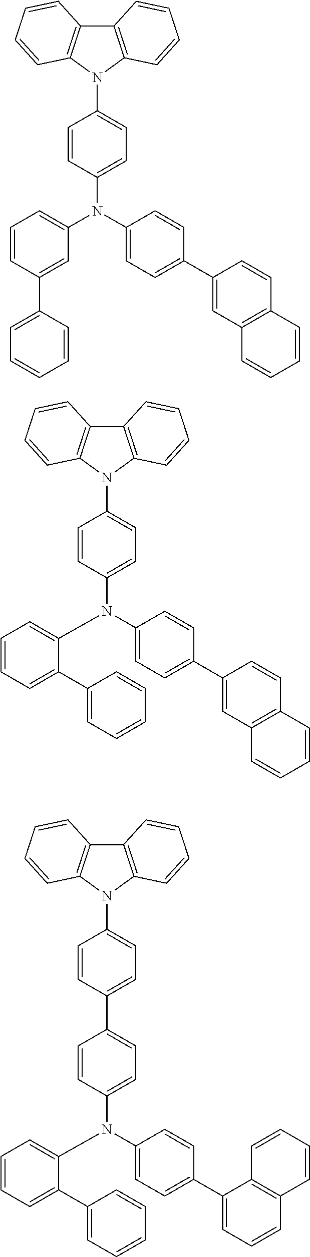

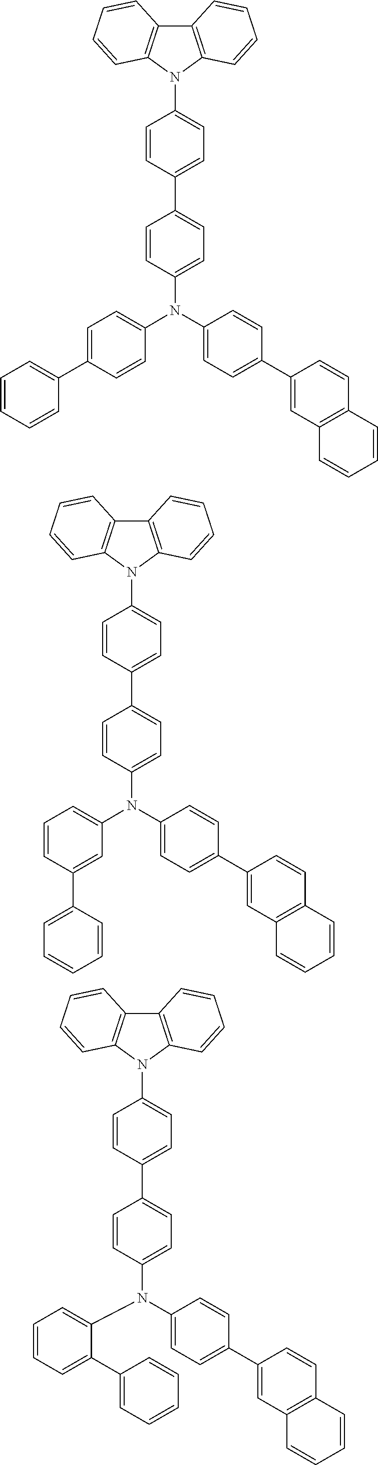

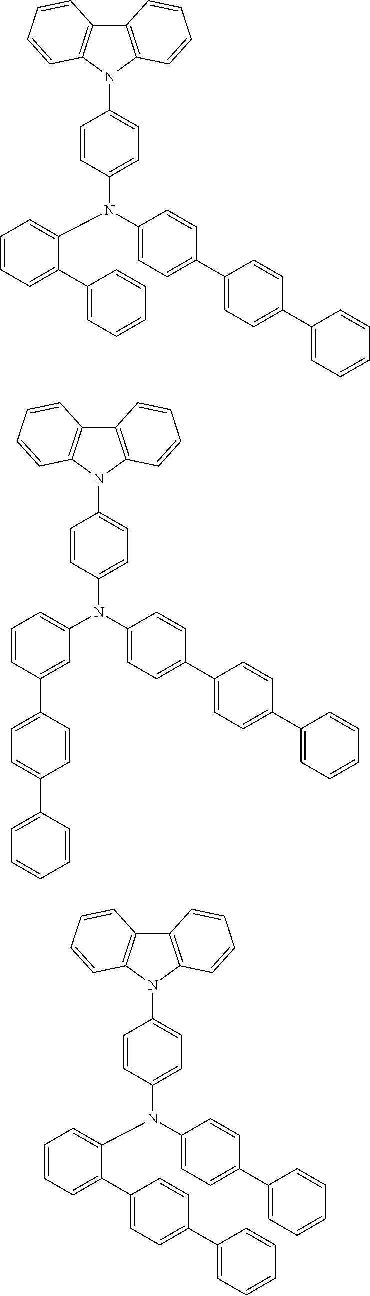 [SMILES: c1ccc(-c2ccc(-c3ccc(N(c4ccc(-n5c6ccccc6c6ccccc65)cc4)c4ccccc4-c4ccccc4)cc3)cc2)cc1 ; c1ccc(-c2ccc(-c3ccc(N(c4ccc(-n5c6ccccc6c6ccccc65)cc4)c4cccc(-c5ccc(-c6ccccc6)cc5)c4)cc3)cc2)cc1 ; c1ccc(-c2ccc(-c3ccccc3N(c3ccc(-c4ccccc4)cc3)c3ccc(-n4c5ccccc5c5ccccc54)cc3)cc2)cc1]

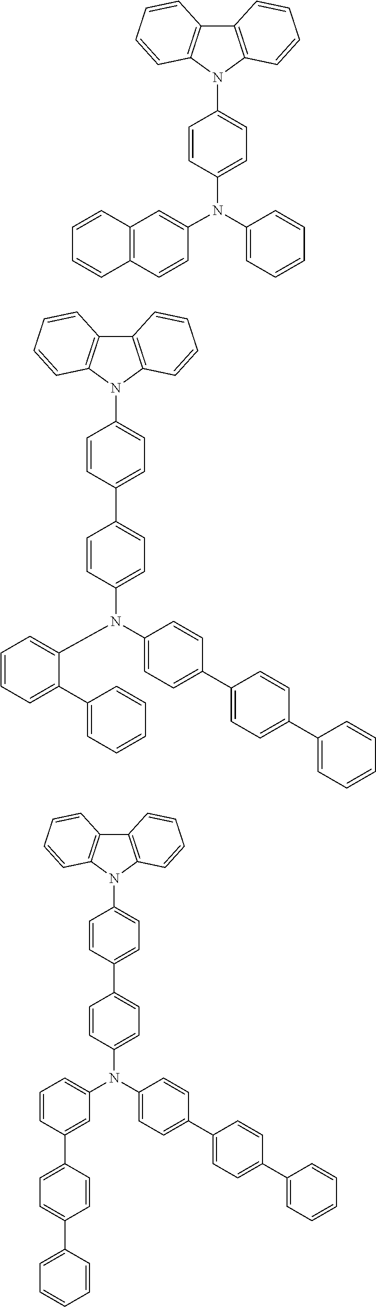

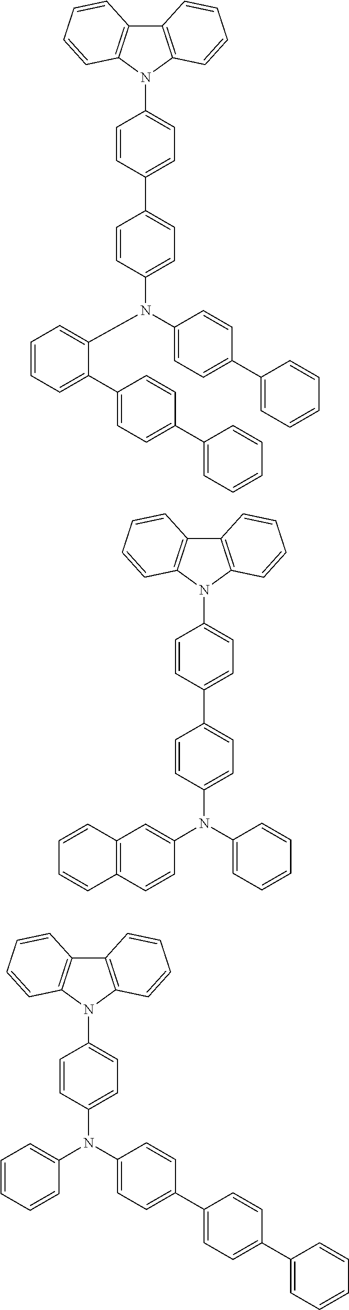

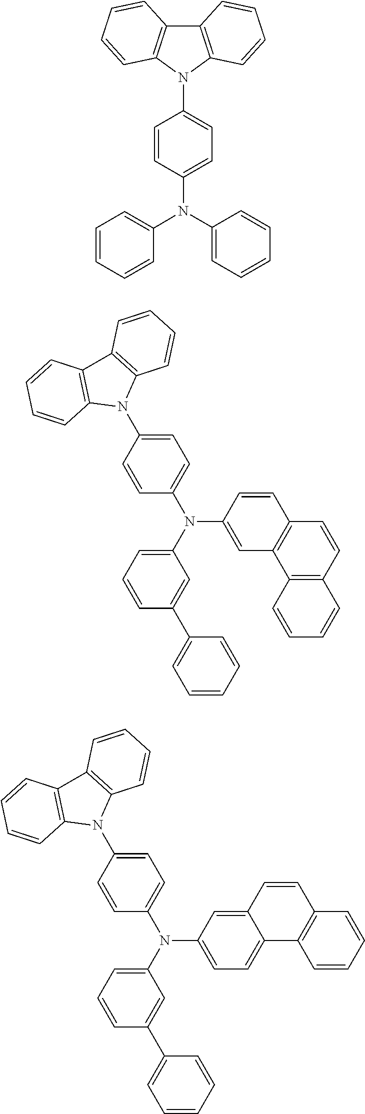

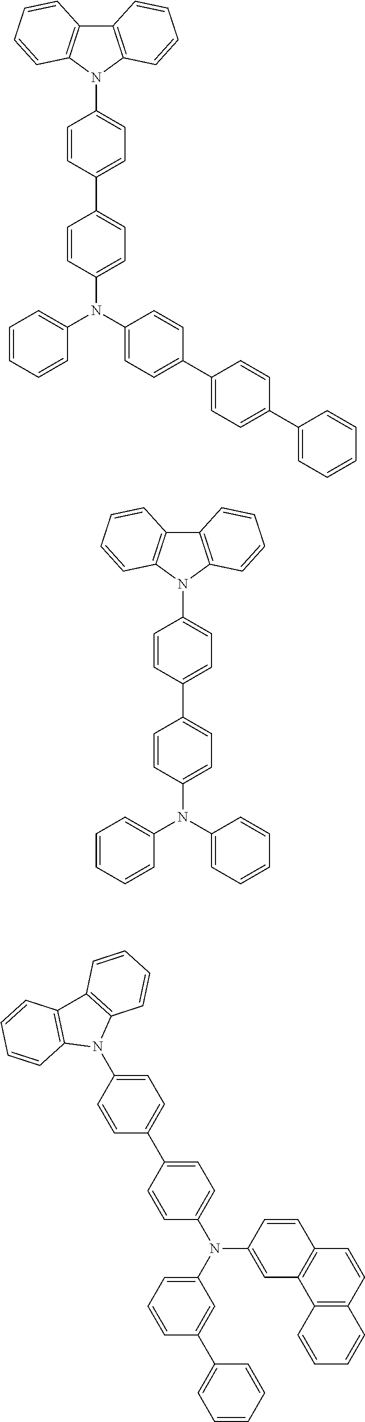 [SMILES: c1ccc(-c2ccc(-c3ccc(N(c4ccccc4)c4ccc(-c5ccc(-n6c7ccccc7c7ccccc76)cc5)cc4)cc3)cc2)cc1 ; c1ccc(N(c2ccccc2)c2ccc(-c3ccc(-n4c5ccccc5c5ccccc54)cc3)cc2)cc1 ; c1ccc(-c2cccc(N(c3ccc(-c4ccc(-n5c6ccccc6c6ccccc65)cc4)cc3)c3ccc4ccc5ccccc5c4c3)c2)cc1]

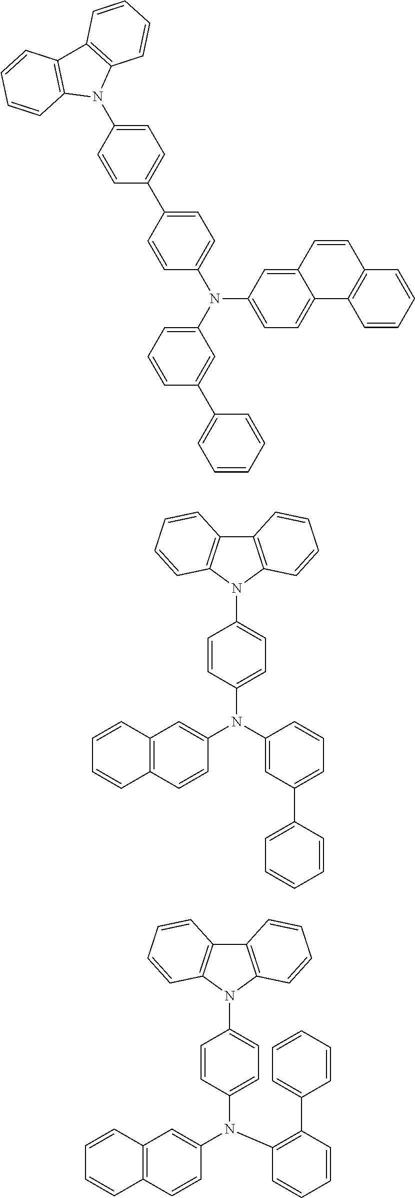

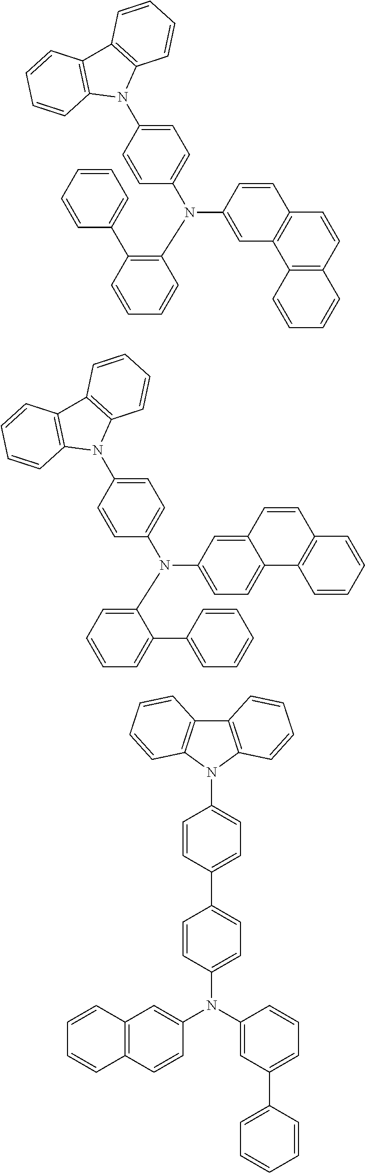

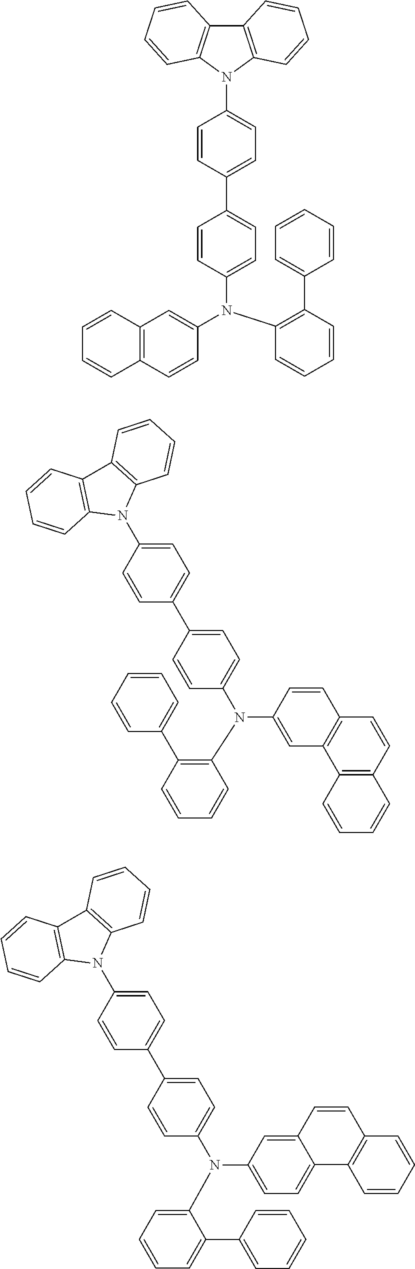

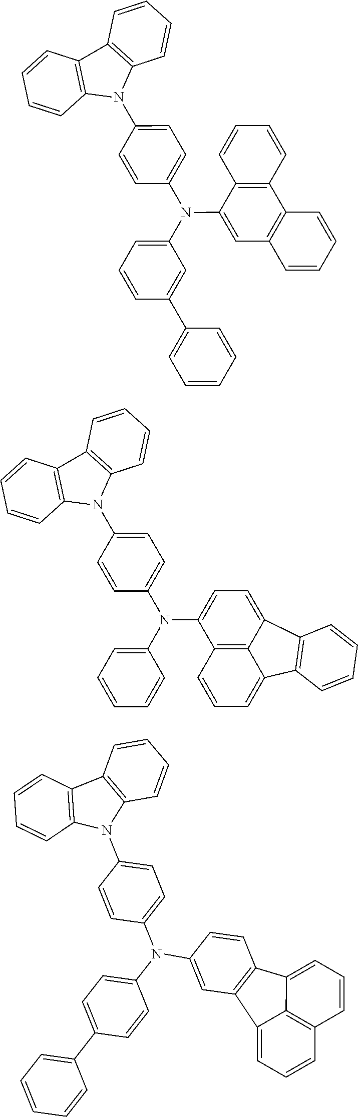

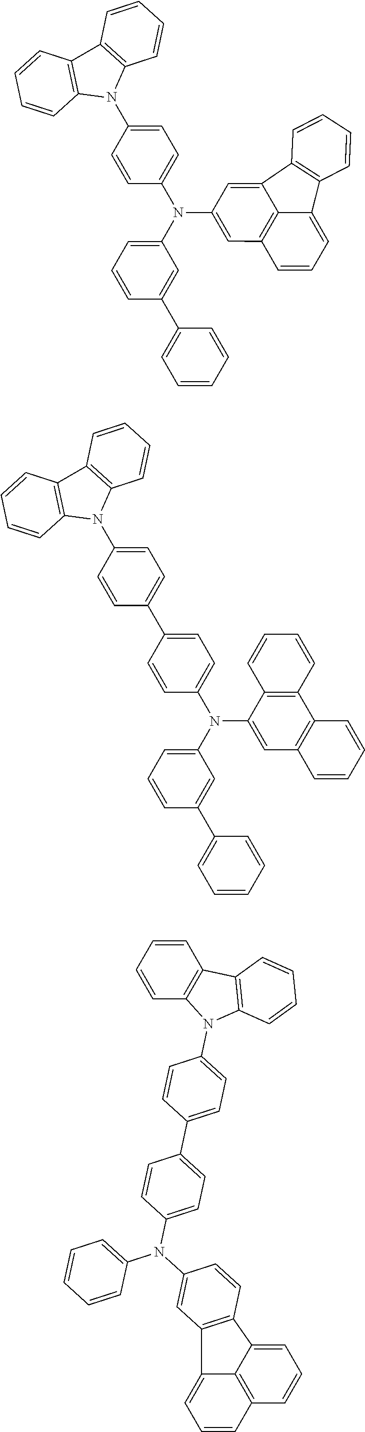 [SMILES: c1ccc(-c2cccc(N(c3ccc(-n4c5ccccc5c5ccccc54)cc3)c3cc4c5c(cccc5c3)-c3ccccc3-4)c2)cc1 ; c1ccc(-c2cccc(N(c3ccc(-c4ccc(-n5c6ccccc6c6ccccc65)cc4)cc3)c3cc4ccccc4c4ccccc34)c2)cc1 ; c1ccc(N(c2ccc(-c3ccc(-n4c5ccccc5c5ccccc54)cc3)cc2)c2ccc3c(c2)-c2cccc4cccc-3c24)cc1]

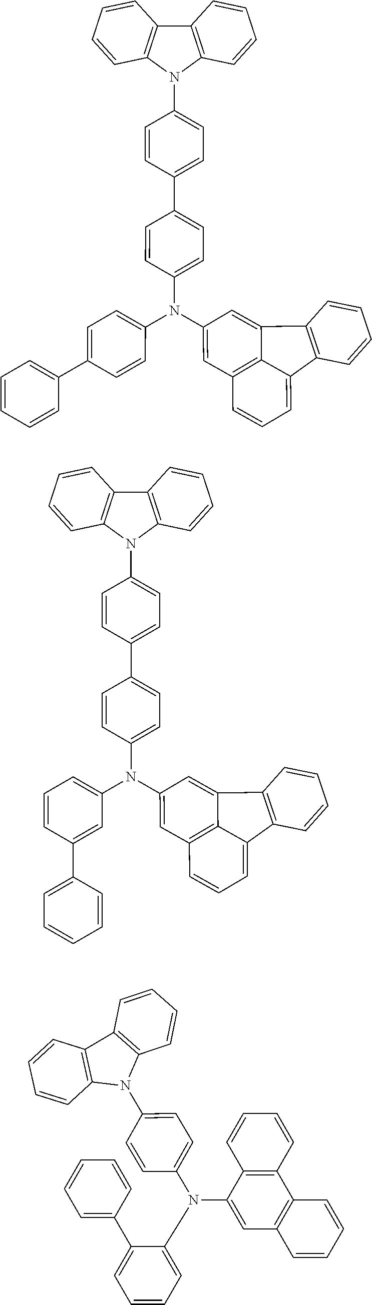 [SMILES: c1ccc(-c2ccc(N(c3ccc(-c4ccc(-n5c6ccccc6c6ccccc65)cc4)cc3)c3cc4c5c(cccc5c3)-c3ccccc3-4)cc2)cc1 ; c1ccc(-c2cccc(N(c3ccc(-c4ccc(-n5c6ccccc6c6ccccc65)cc4)cc3)c3cc4c5c(cccc5c3)-c3ccccc3-4)c2)cc1 ; c1ccc(-c2ccccc2N(c2ccc(-n3c4ccccc4c4ccccc43)cc2)c2cc3ccccc3c3ccccc23)cc1]

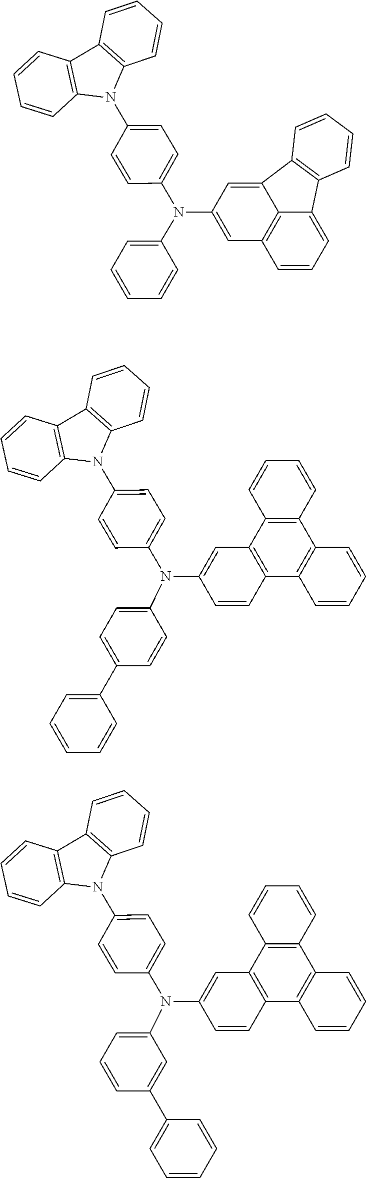

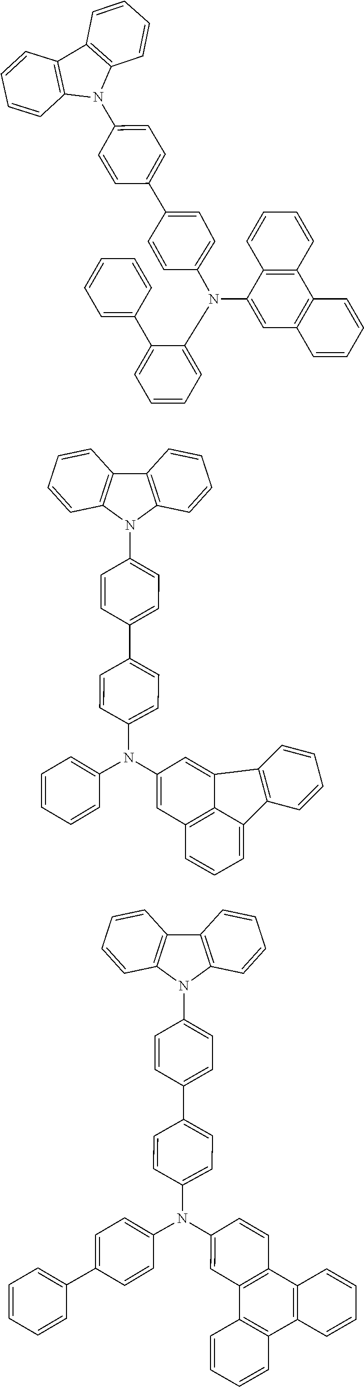

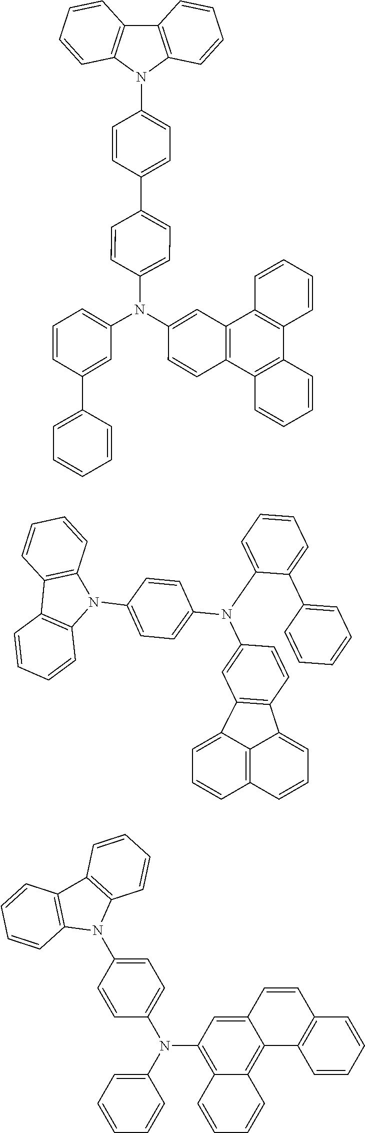

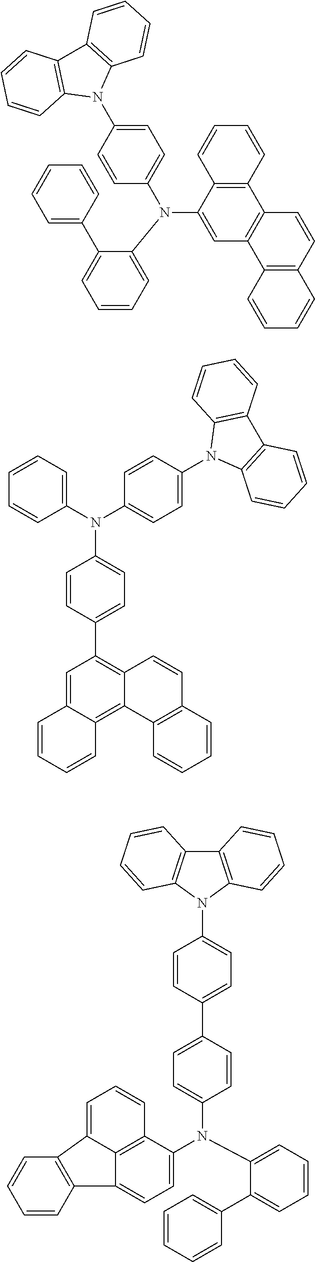

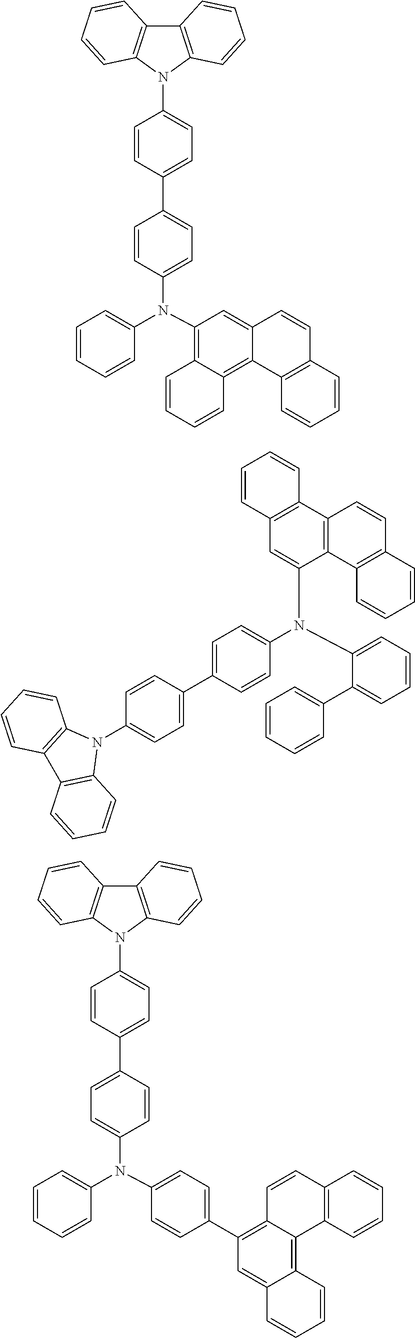

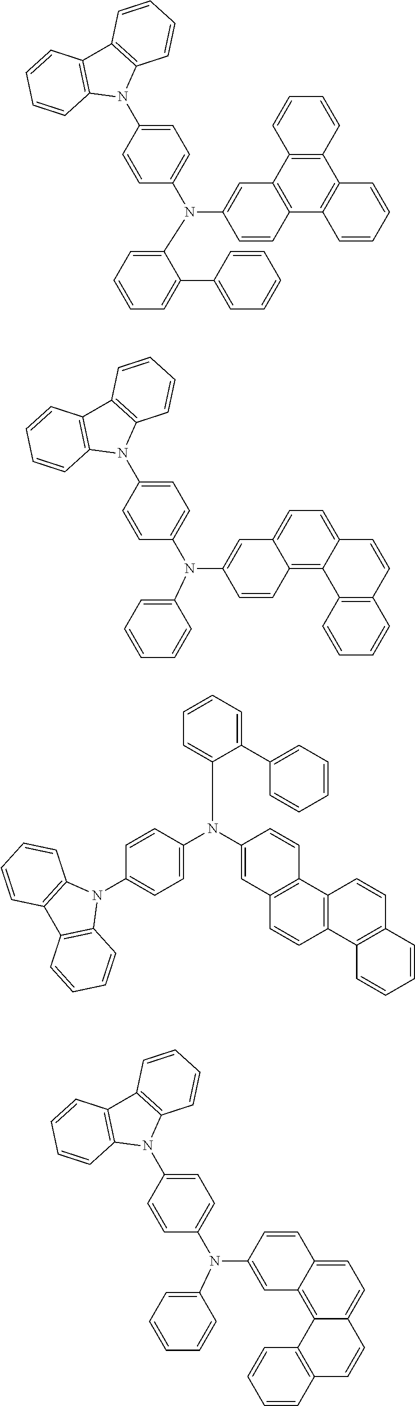 [SMILES: c1ccc(-c2ccccc2N(c2ccc(-n3c4ccccc4c4ccccc43)cc2)c2ccc3c4ccccc4c4ccccc4c3c2)cc1 ; c1ccc(N(c2ccc(-n3c4ccccc4c4ccccc43)cc2)c2ccc3c(ccc4ccc5ccccc5c43)c2)cc1 ; c1ccc(-c2ccccc2N(c2ccc(-n3c4ccccc4c4ccccc43)cc2)c2ccc3c(ccc4c5ccccc5ccc34)c2)cc1 ; c1ccc(N(c2ccc(-n3c4ccccc4c4ccccc43)cc2)c2ccc3ccc4ccc5ccccc5c4c3c2)cc1]

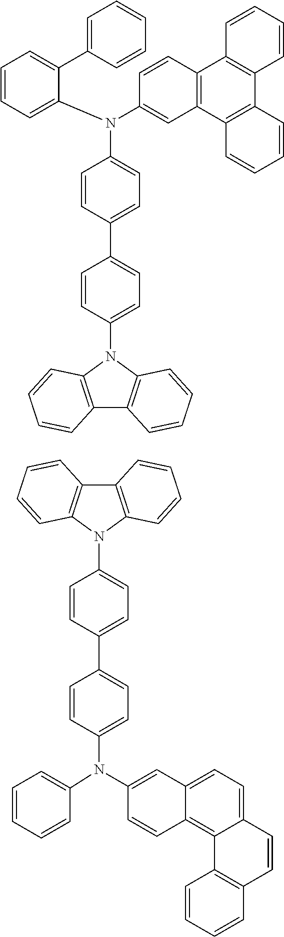

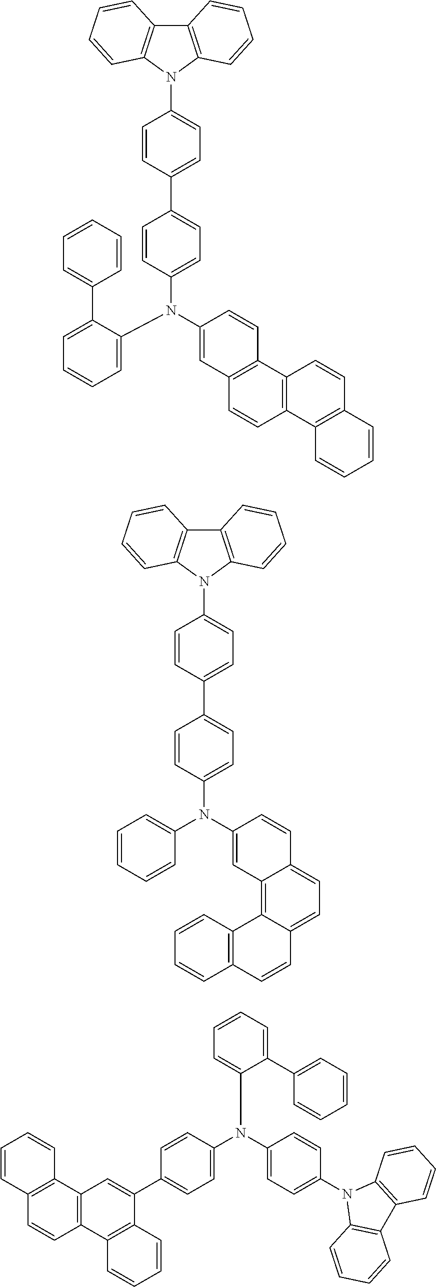

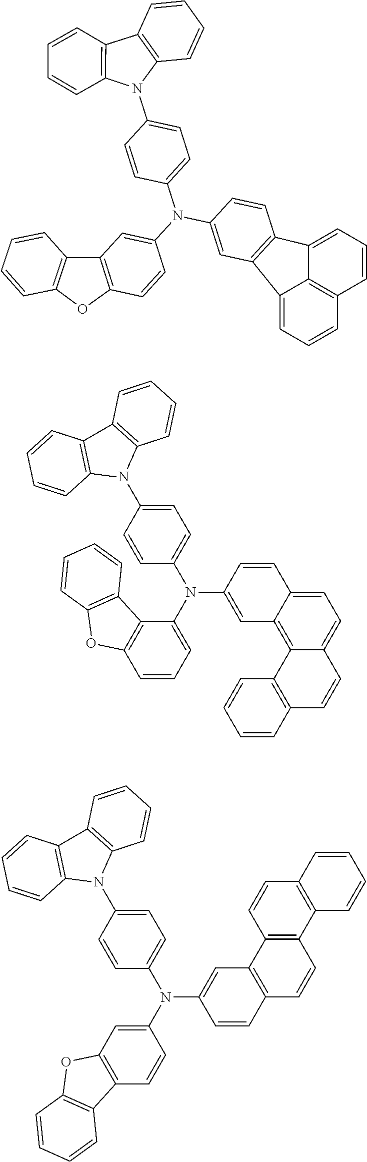 [SMILES: c1cc2c3c(cccc3c1)-c1cc(N(c3ccc(-n4c5ccccc5c5ccccc54)cc3)c3ccc4oc5ccccc5c4c3)ccc1-2 ; c1ccc2c(c1)ccc1ccc3ccc(N(c4ccc(-n5c6ccccc6c6ccccc65)cc4)c4cccc5oc6ccccc6c45)cc3c12 ; c1ccc2c(c1)ccc1c3cc(N(c4ccc(-n5c6ccccc6c6ccccc65)cc4)c4ccc5c(c4)oc4ccccc45)ccc3ccc21]

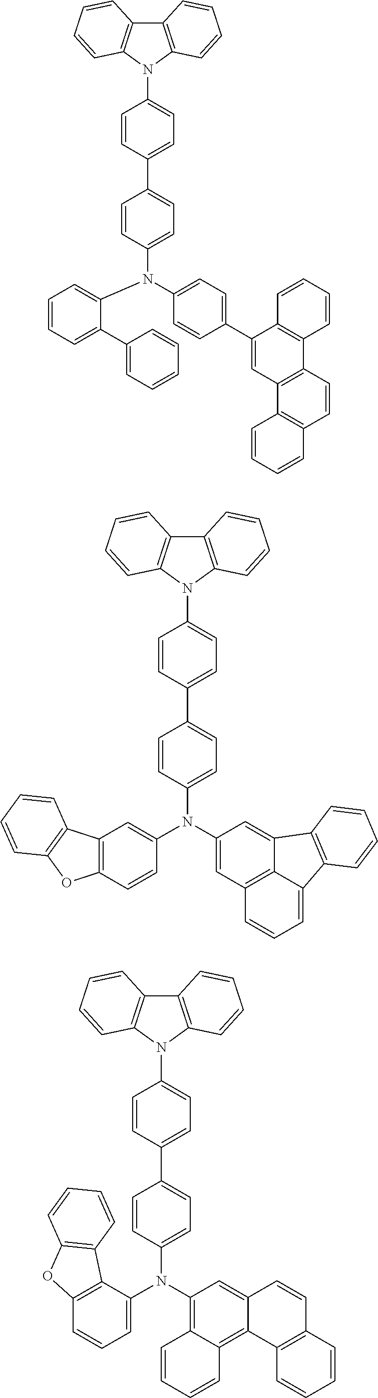 [SMILES: c1ccc(-c2ccccc2N(c2ccc(-c3ccc(-n4c5ccccc5c5ccccc54)cc3)cc2)c2ccc(-c3cc4c5ccccc5ccc4c4ccccc34)cc2)cc1 ; c1ccc2c(c1)-c1cccc3cc(N(c4ccc(-c5ccc(-n6c7ccccc7c7ccccc76)cc5)cc4)c4ccc5oc6ccccc6c5c4)cc-2c13 ; c1ccc2c(c1)ccc1cc(N(c3ccc(-c4ccc(-n5c6ccccc6c6ccccc65)cc4)cc3)c3cccc4oc5ccccc5c34)c3ccccc3c12]

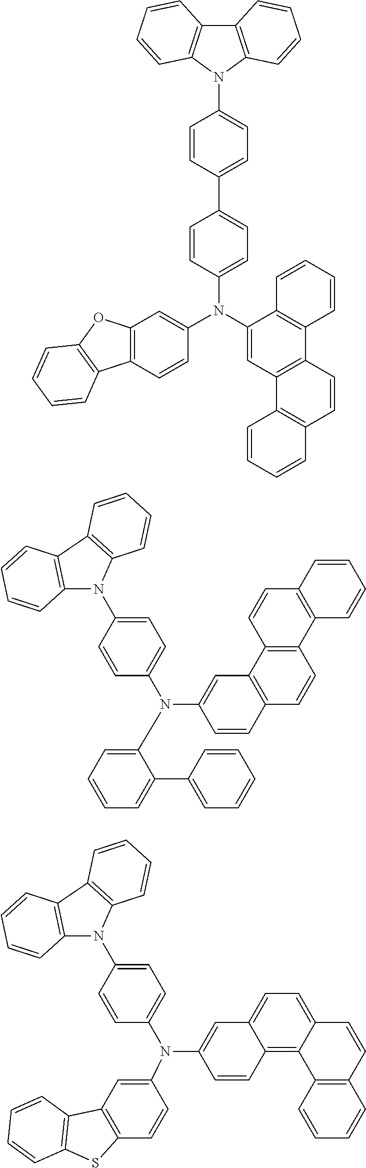

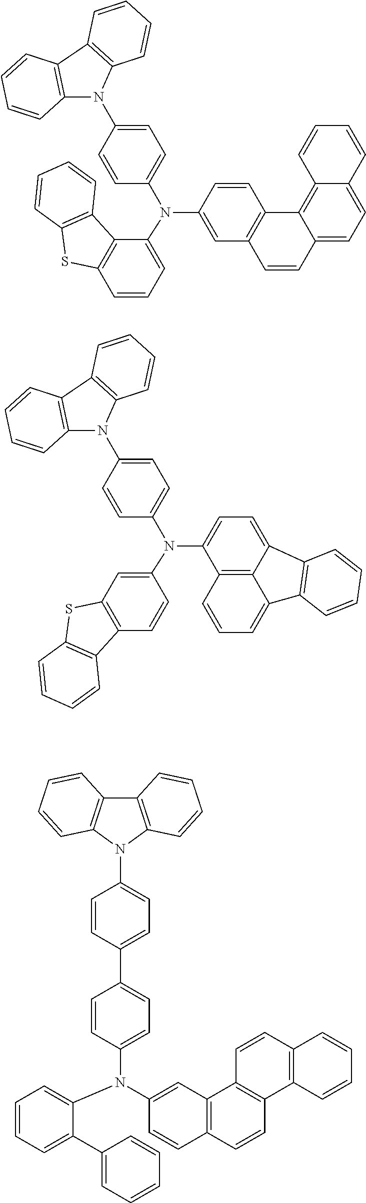

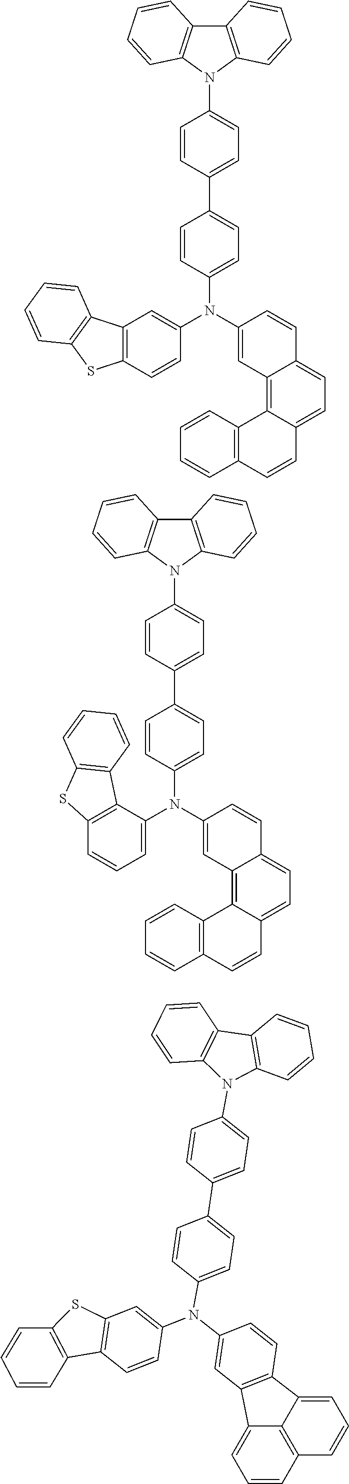

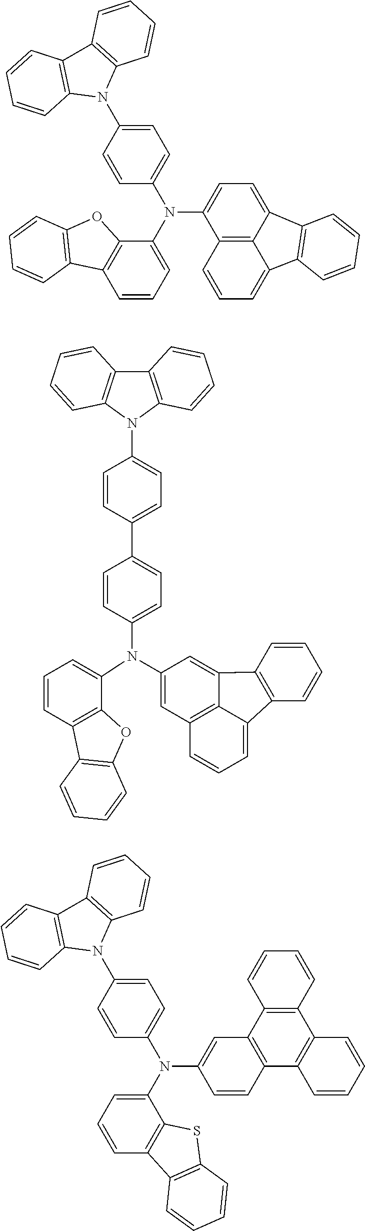

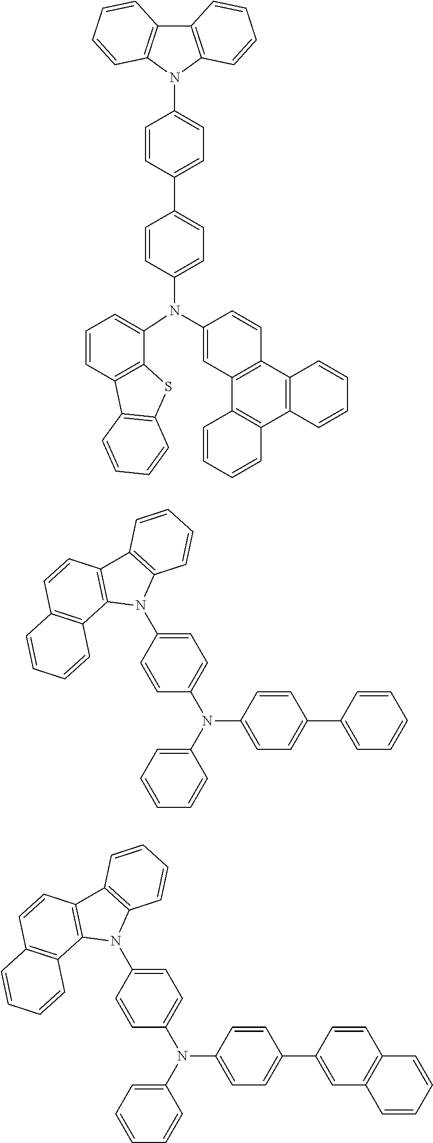

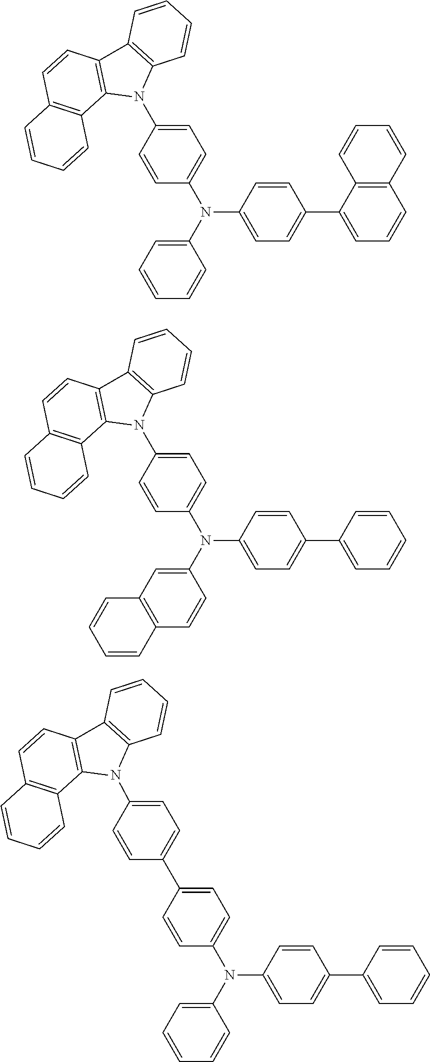

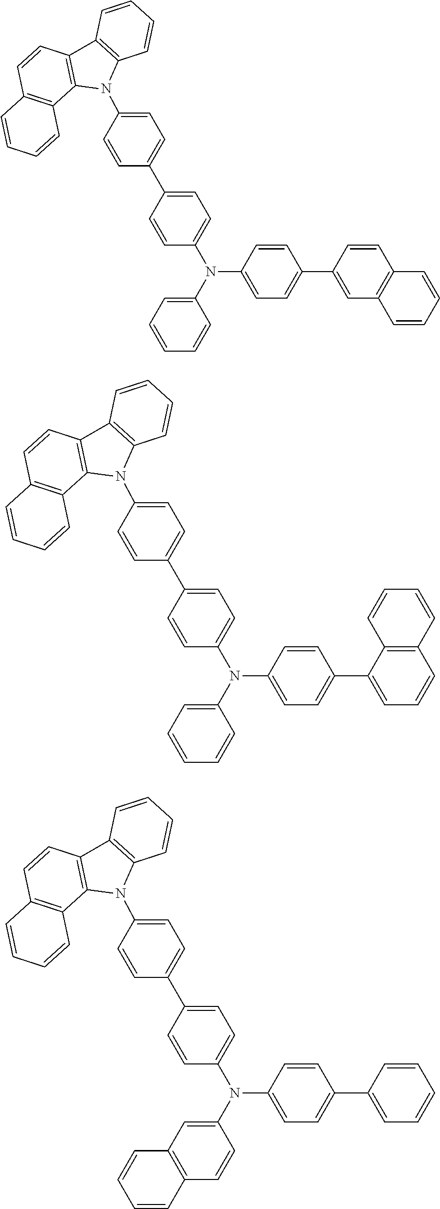

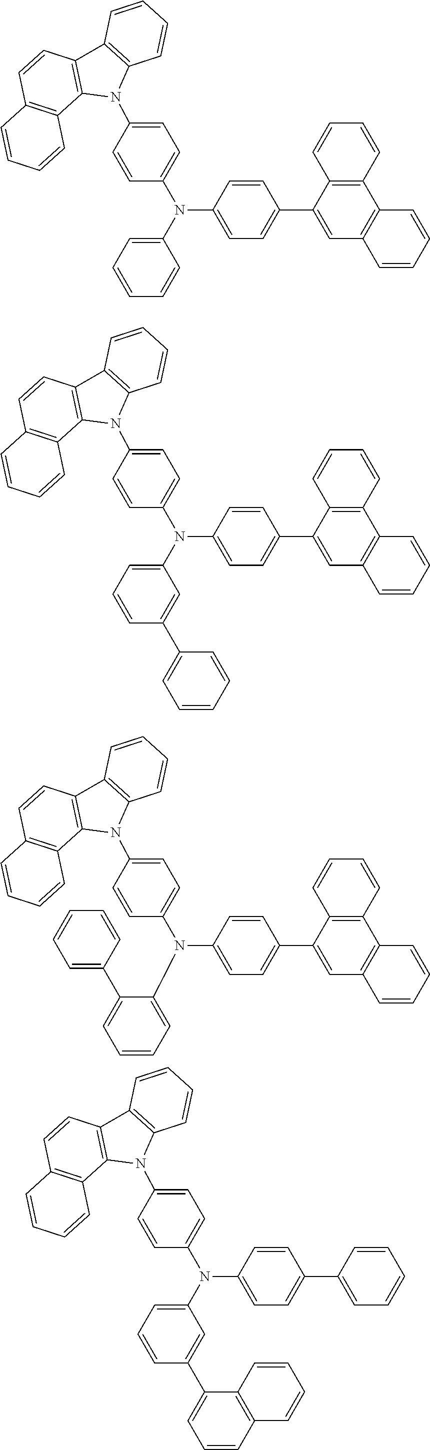

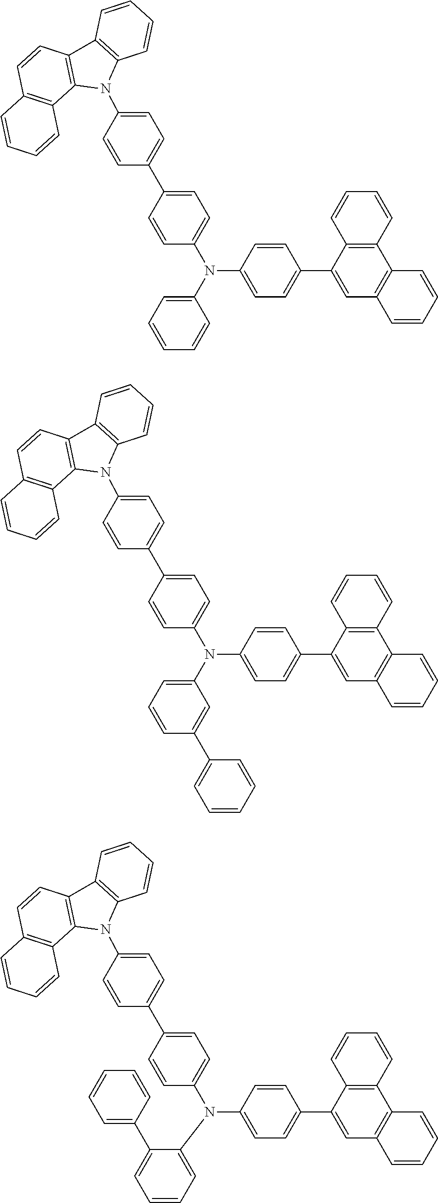 [SMILES: c1ccc(N(c2ccc(-c3ccc(-n4c5ccccc5c5ccc6ccccc6c54)cc3)cc2)c2ccc(-c3cc4ccccc4c4ccccc34)cc2)cc1 ; c1ccc(-c2cccc(N(c3ccc(-c4ccc(-n5c6ccccc6c6ccc7ccccc7c65)cc4)cc3)c3ccc(-c4cc5ccccc5c5ccccc45)cc3)c2)cc1 ; c1ccc(-c2ccccc2N(c2ccc(-c3ccc(-n4c5ccccc5c5ccc6ccccc6c54)cc3)cc2)c2ccc(-c3cc4ccccc4c4ccccc34)cc2)cc1]

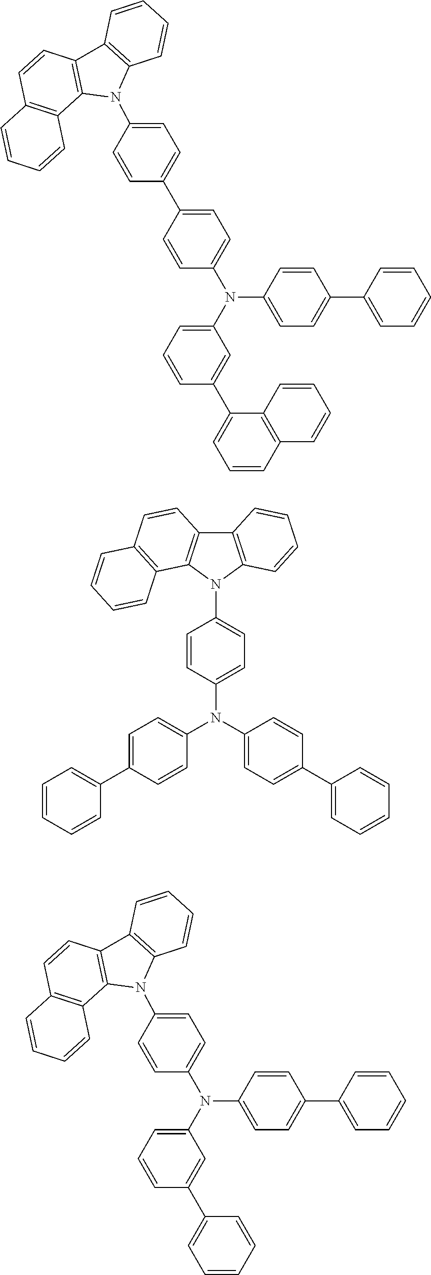

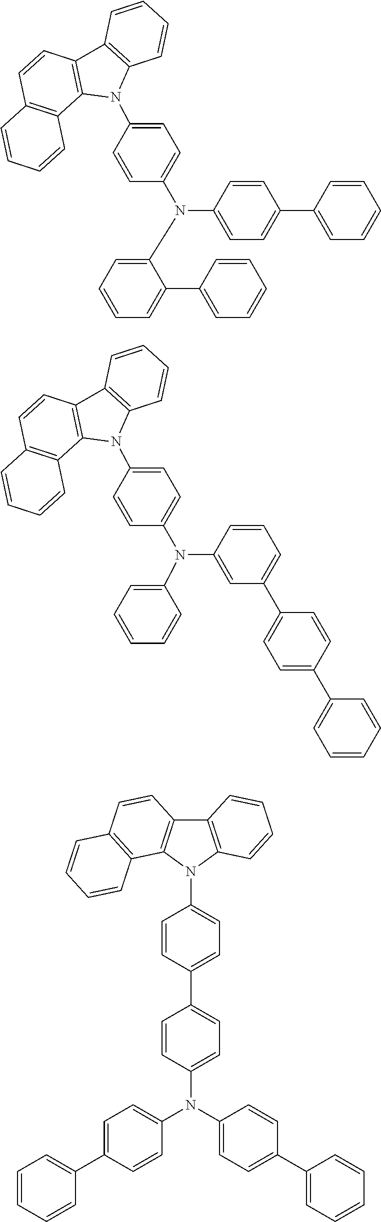

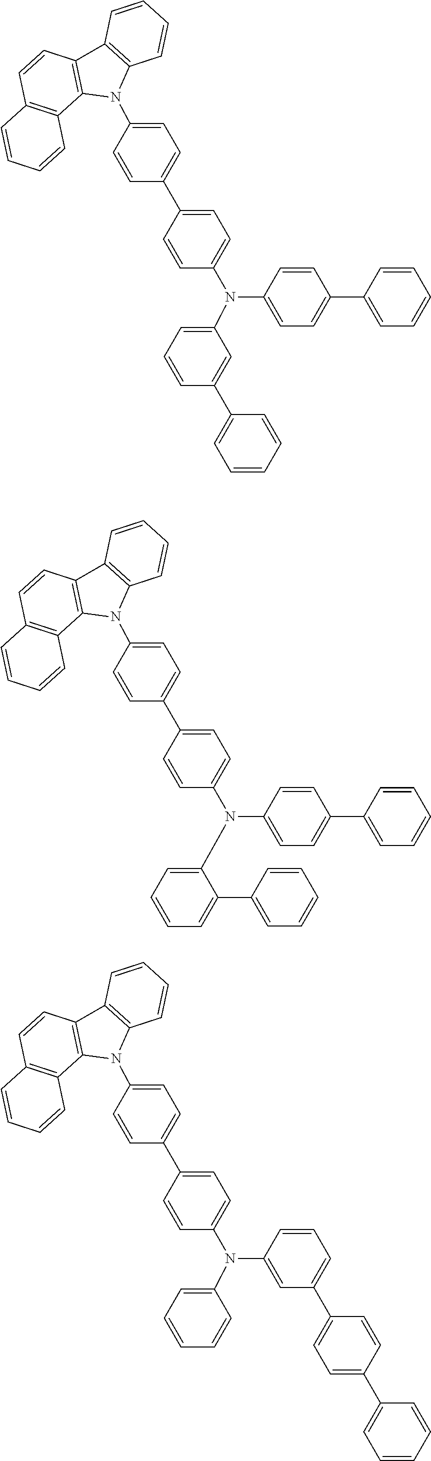

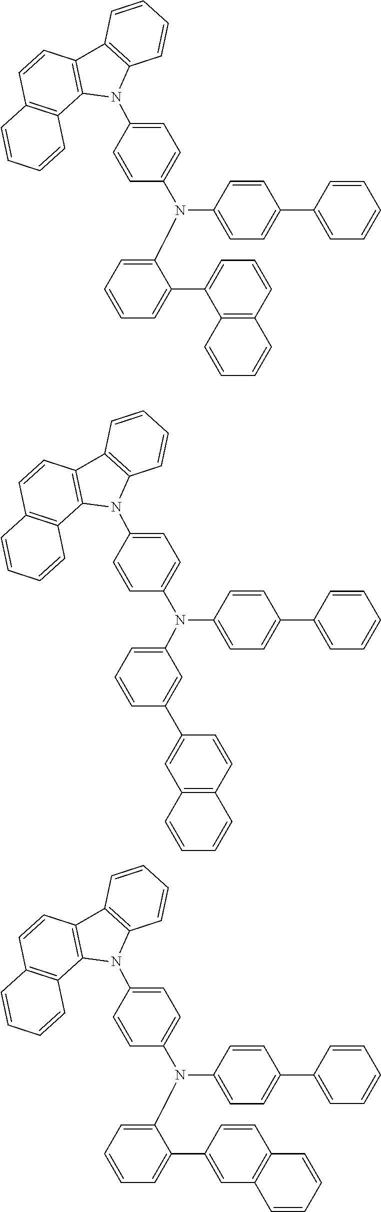

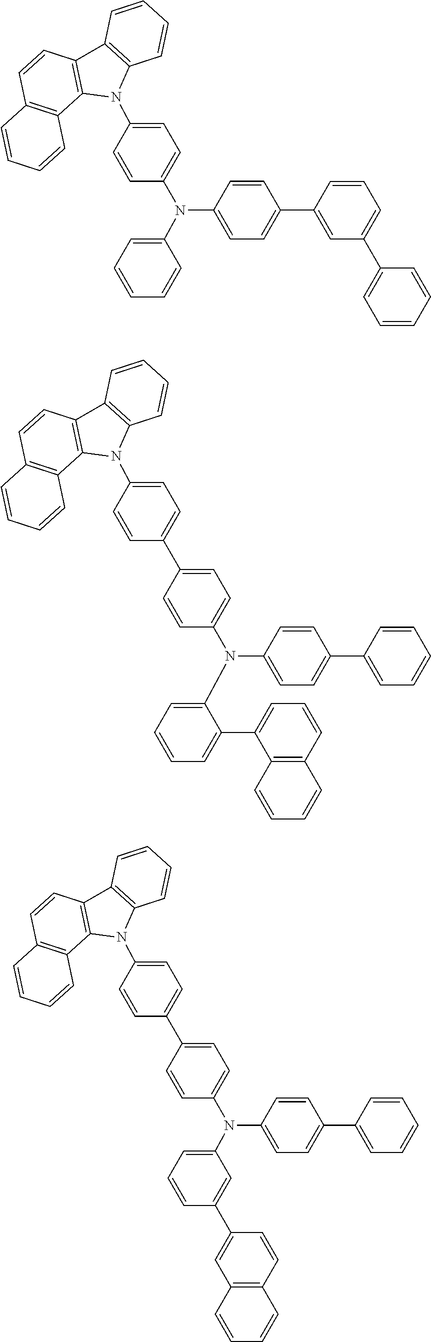 [SMILES: c1ccc(-c2cccc(-c3ccc(N(c4ccccc4)c4ccc(-n5c6ccccc6c6ccc7ccccc7c65)cc4)cc3)c2)cc1 ; c1ccc(-c2ccc(N(c3ccc(-c4ccc(-n5c6ccccc6c6ccc7ccccc7c65)cc4)cc3)c3ccccc3-c3cccc4ccccc34)cc2)cc1 ; c1ccc(-c2ccc(N(c3ccc(-c4ccc(-n5c6ccccc6c6ccc7ccccc7c65)cc4)cc3)c3cccc(-c4ccc5ccccc5c4)c3)cc2)cc1]

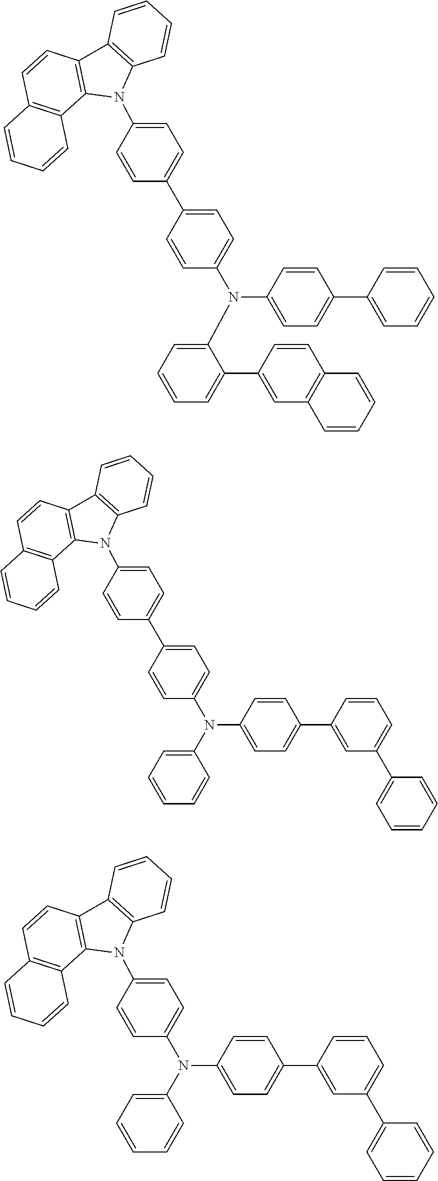 [SMILES: c1ccc(-c2ccc(N(c3ccc(-c4ccc(-n5c6ccccc6c6ccc7ccccc7c65)cc4)cc3)c3ccccc3-c3ccc4ccccc4c3)cc2)cc1 ; c1ccc(-c2cccc(-c3ccc(N(c4ccccc4)c4ccc(-c5ccc(-n6c7ccccc7c7ccc8ccccc8c76)cc5)cc4)cc3)c2)cc1 ; c1ccc(-c2cccc(-c3ccc(N(c4ccccc4)c4ccc(-n5c6ccccc6c6ccc7ccccc7c65)cc4)cc3)c2)cc1]

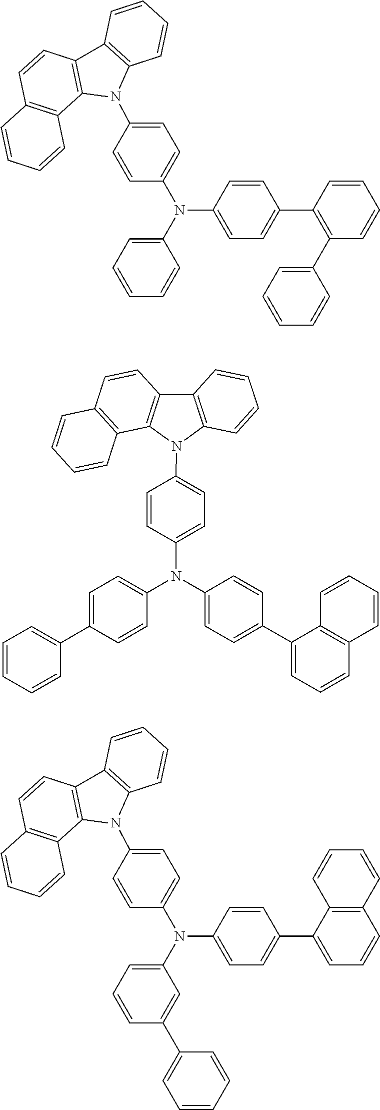 [SMILES: c1ccc(-c2ccccc2-c2ccc(N(c3ccccc3)c3ccc(-n4c5ccccc5c5ccc6ccccc6c54)cc3)cc2)cc1 ; c1ccc(-c2ccc(N(c3ccc(-c4cccc5ccccc45)cc3)c3ccc(-n4c5ccccc5c5ccc6ccccc6c54)cc3)cc2)cc1 ; c1ccc(-c2cccc(N(c3ccc(-c4cccc5ccccc45)cc3)c3ccc(-n4c5ccccc5c5ccc6ccccc6c54)cc3)c2)cc1]

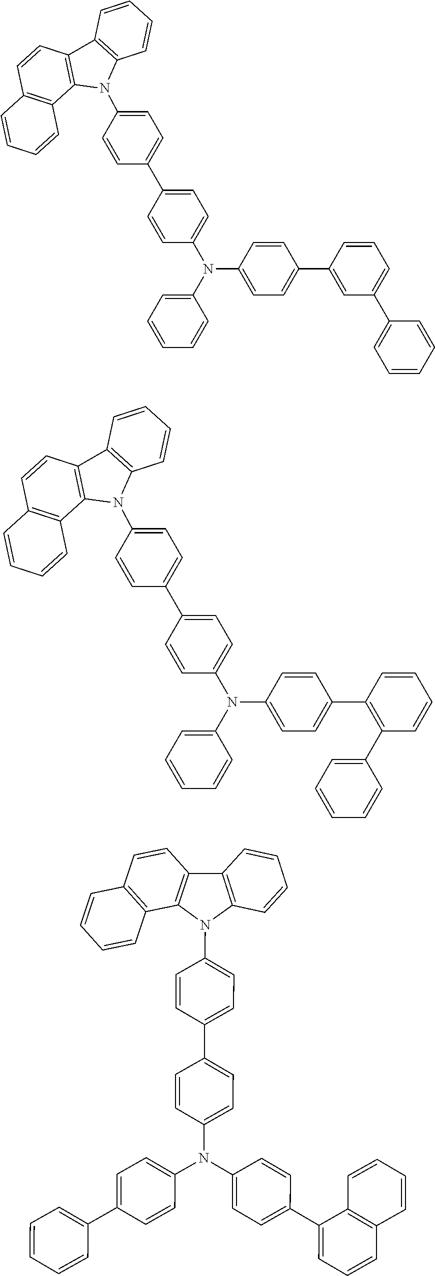 [SMILES: c1ccc(-c2cccc(-c3ccc(N(c4ccccc4)c4ccc(-c5ccc(-n6c7ccccc7c7ccc8ccccc8c76)cc5)cc4)cc3)c2)cc1 ; c1ccc(-c2ccccc2-c2ccc(N(c3ccccc3)c3ccc(-c4ccc(-n5c6ccccc6c6ccc7ccccc7c65)cc4)cc3)cc2)cc1 ; c1ccc(-c2ccc(N(c3ccc(-c4ccc(-n5c6ccccc6c6ccc7ccccc7c65)cc4)cc3)c3ccc(-c4cccc5ccccc45)cc3)cc2)cc1]

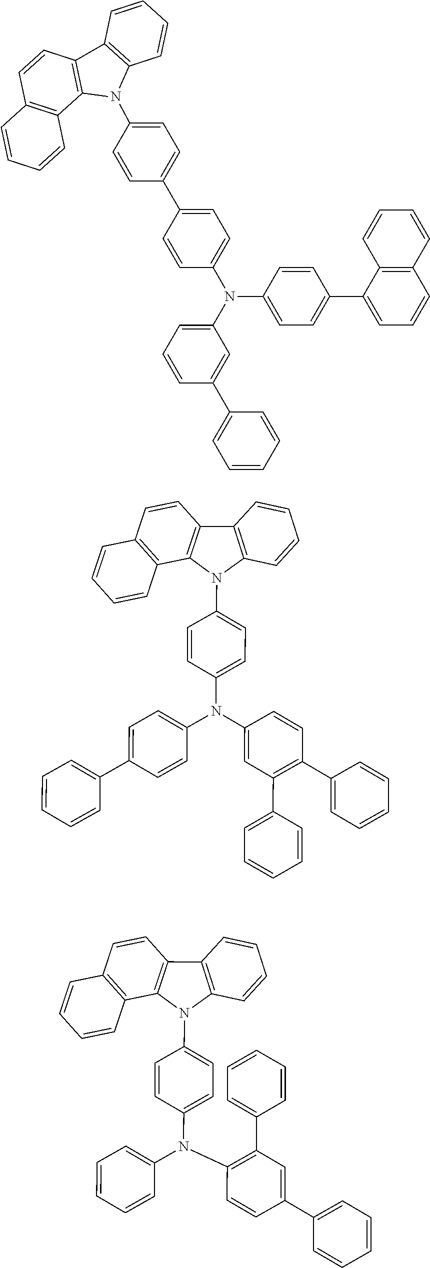

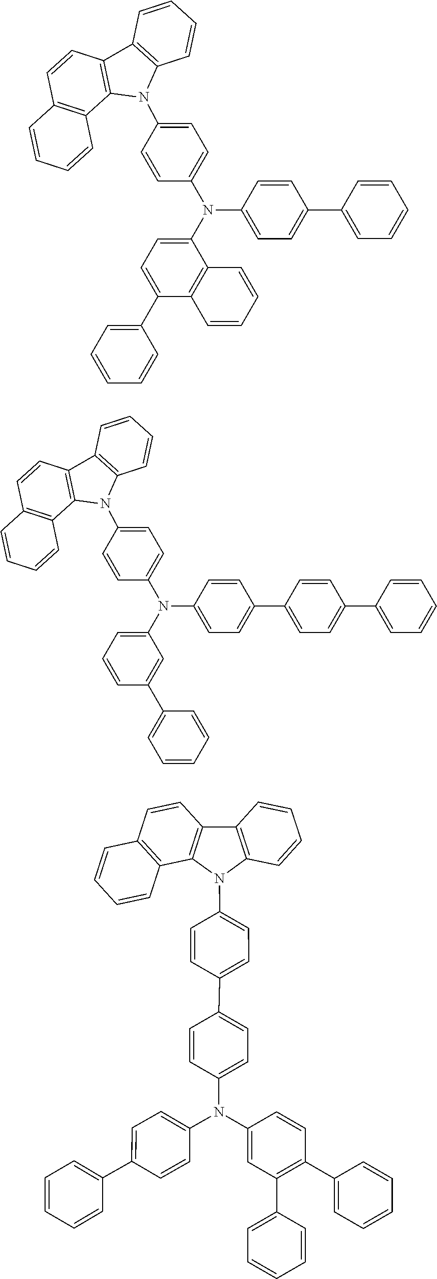

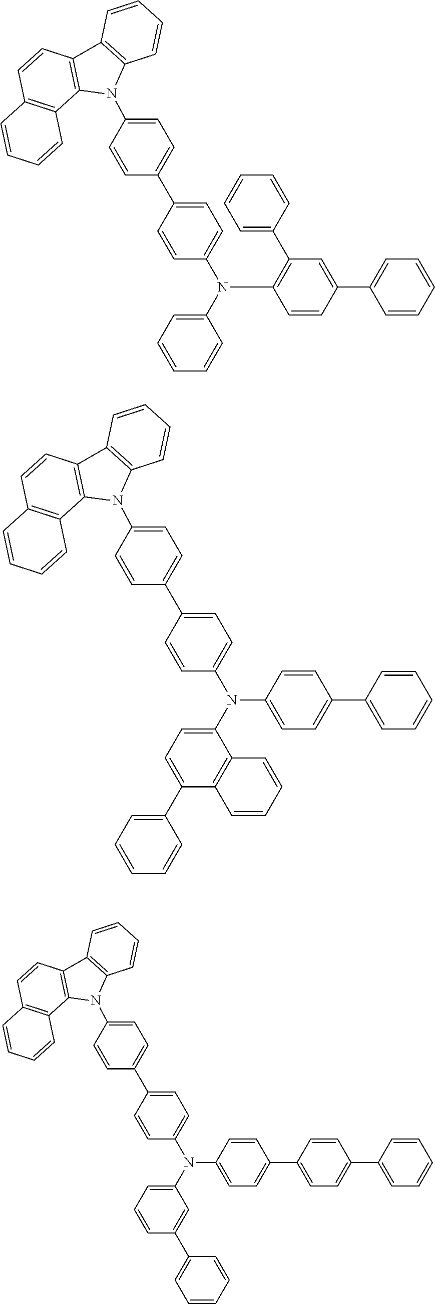

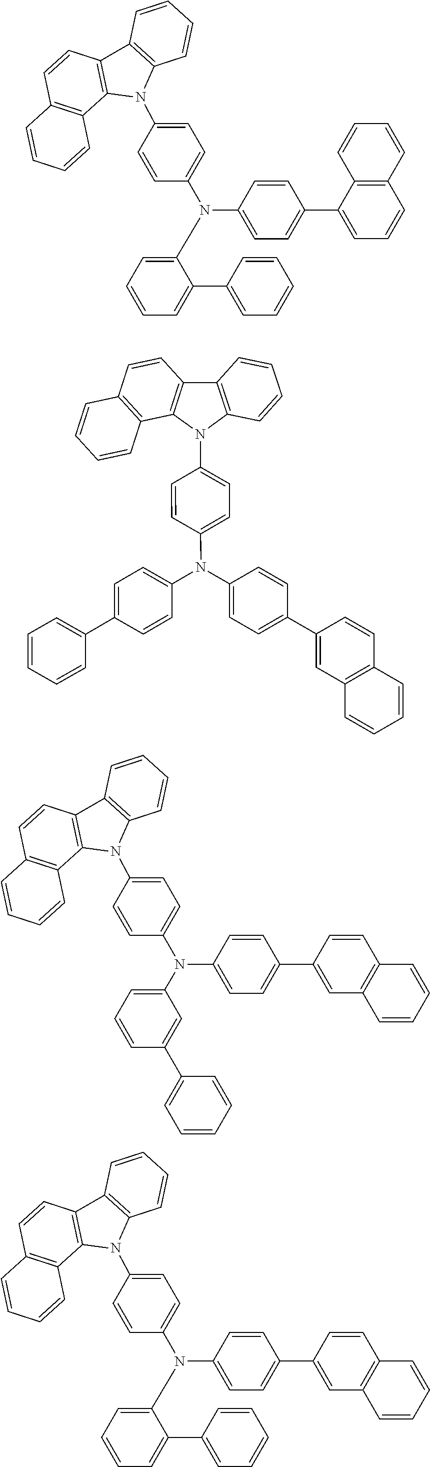

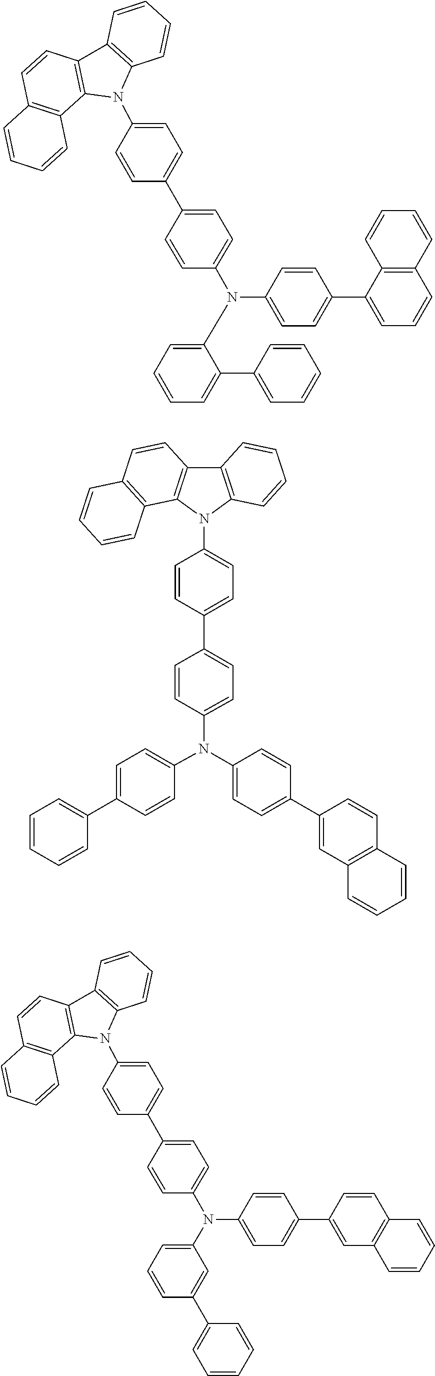

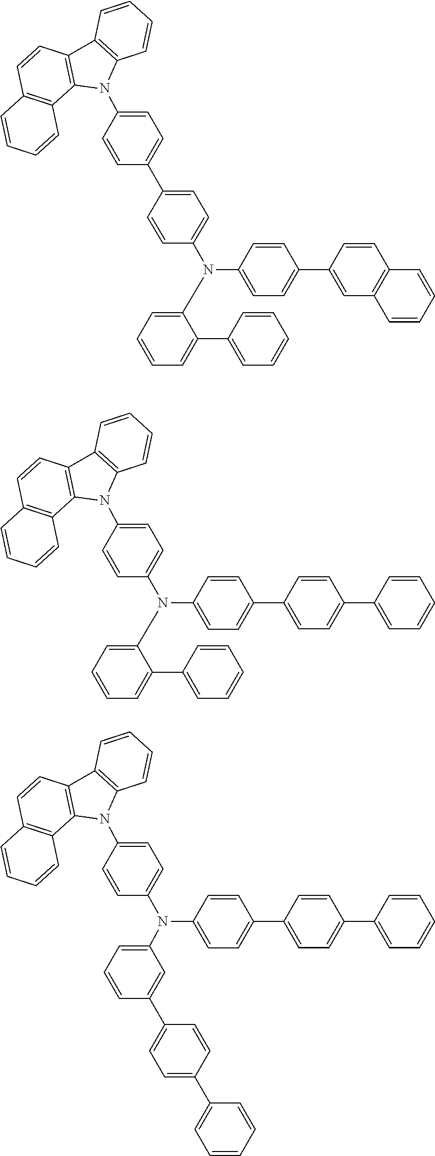

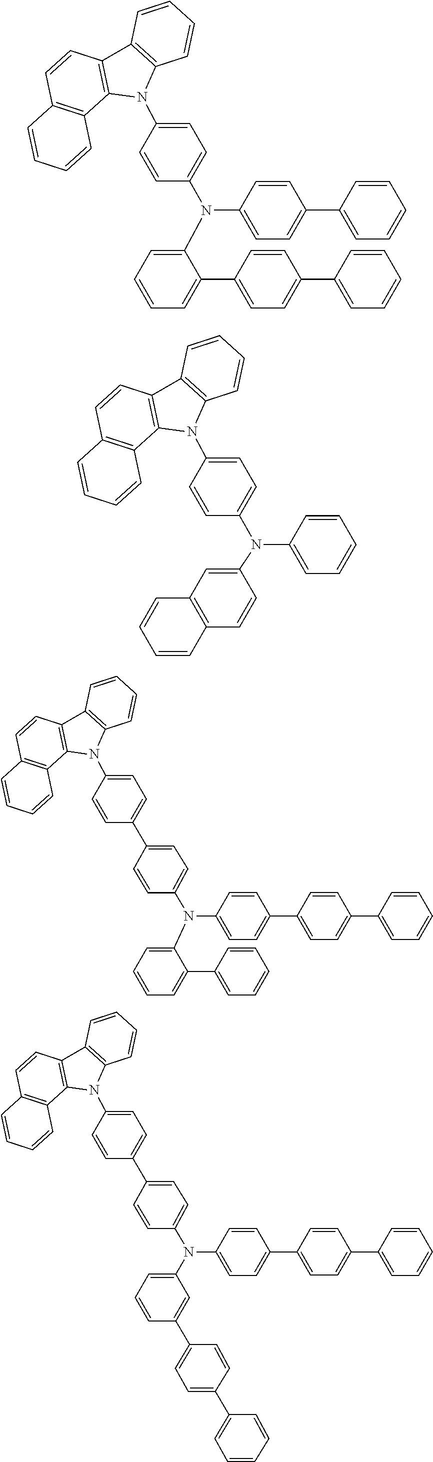

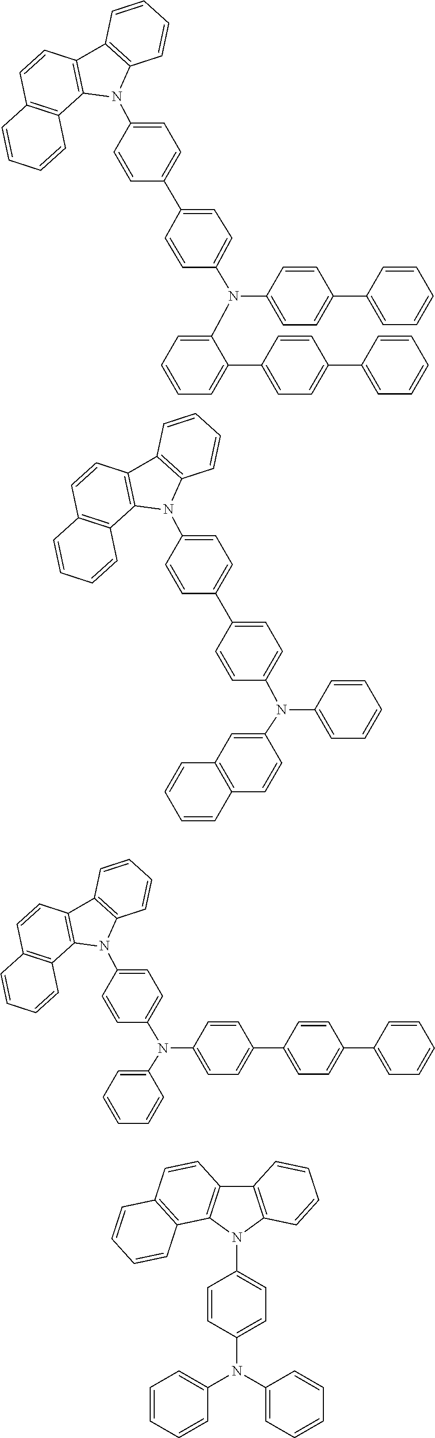

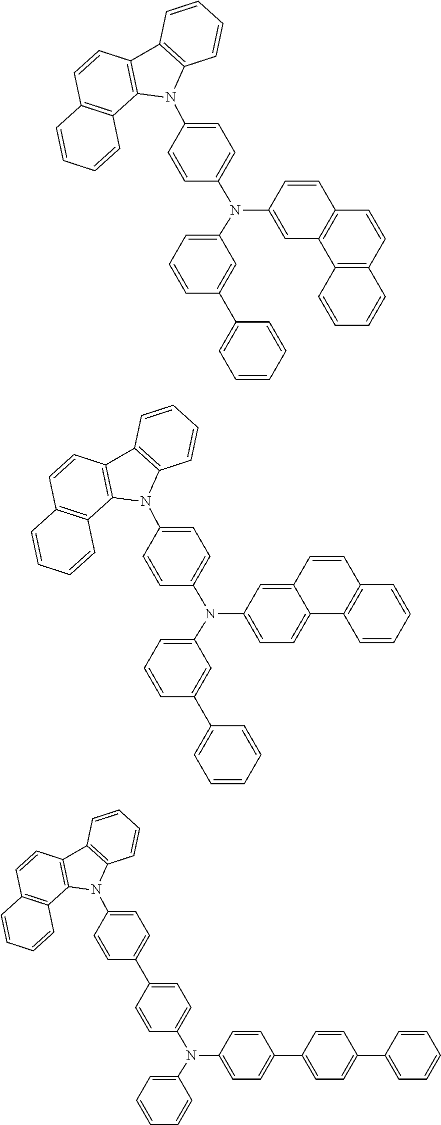 [SMILES: c1ccc(-c2cccc(N(c3ccc(-n4c5ccccc5c5ccc6ccccc6c54)cc3)c3ccc4ccc5ccccc5c4c3)c2)cc1 ; c1ccc(-c2cccc(N(c3ccc(-n4c5ccccc5c5ccc6ccccc6c54)cc3)c3ccc4c(ccc5ccccc54)c3)c2)cc1 ; c1ccc(-c2ccc(-c3ccc(N(c4ccccc4)c4ccc(-c5ccc(-n6c7ccccc7c7ccc8ccccc8c76)cc5)cc4)cc3)cc2)cc1]

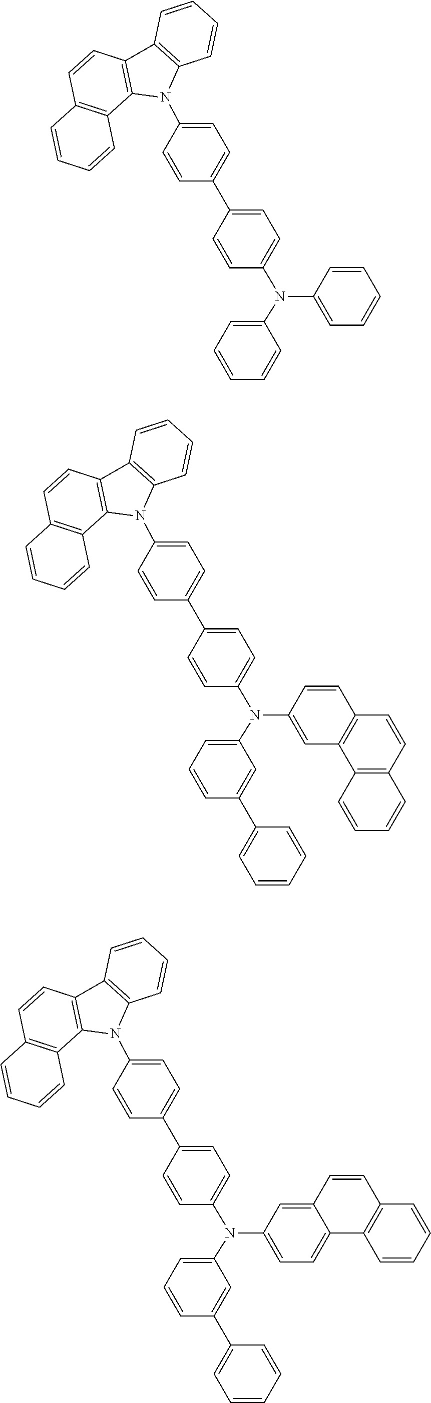 [SMILES: c1ccc(N(c2ccccc2)c2ccc(-c3ccc(-n4c5ccccc5c5ccc6ccccc6c54)cc3)cc2)cc1 ; c1ccc(-c2cccc(N(c3ccc(-c4ccc(-n5c6ccccc6c6ccc7ccccc7c65)cc4)cc3)c3ccc4ccc5ccccc5c4c3)c2)cc1 ; c1ccc(-c2cccc(N(c3ccc(-c4ccc(-n5c6ccccc6c6ccc7ccccc7c65)cc4)cc3)c3ccc4c(ccc5ccccc54)c3)c2)cc1]

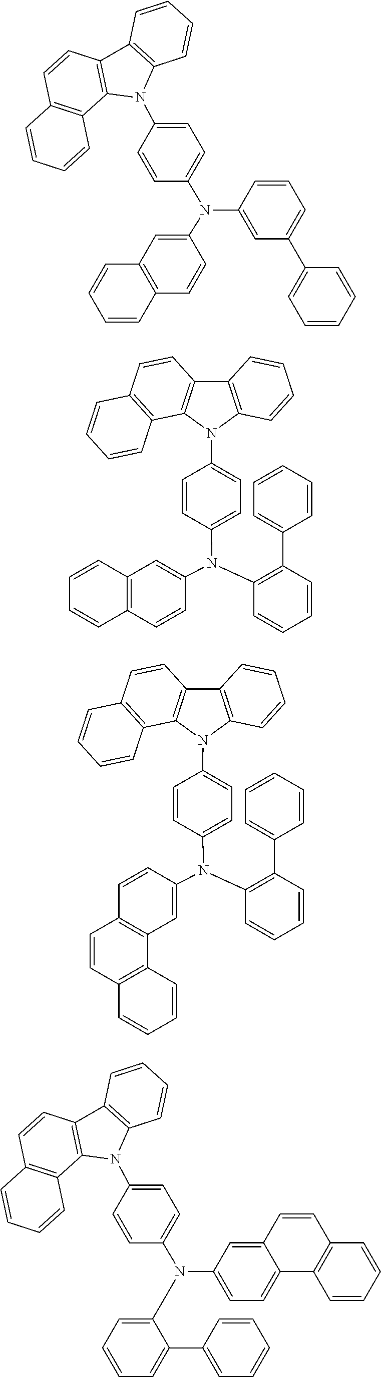

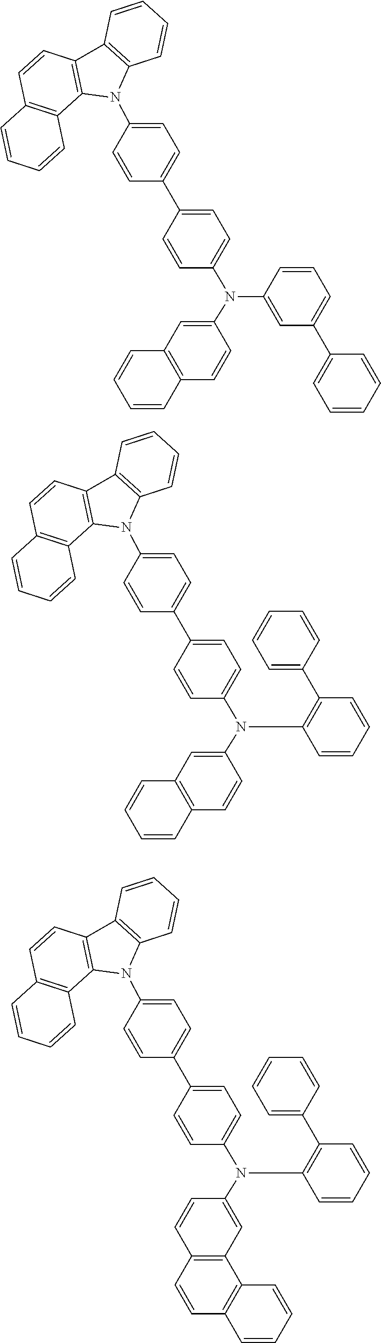

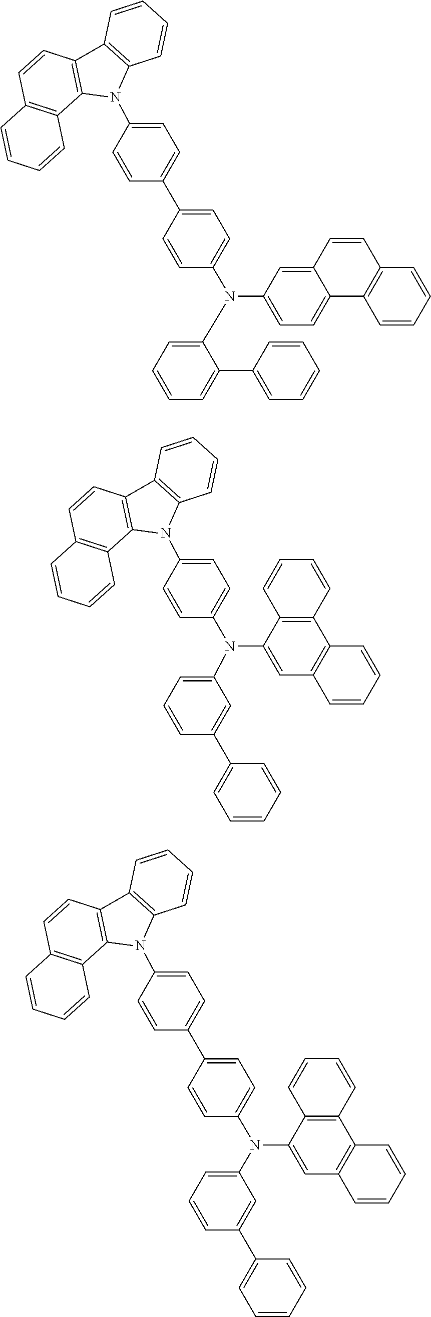

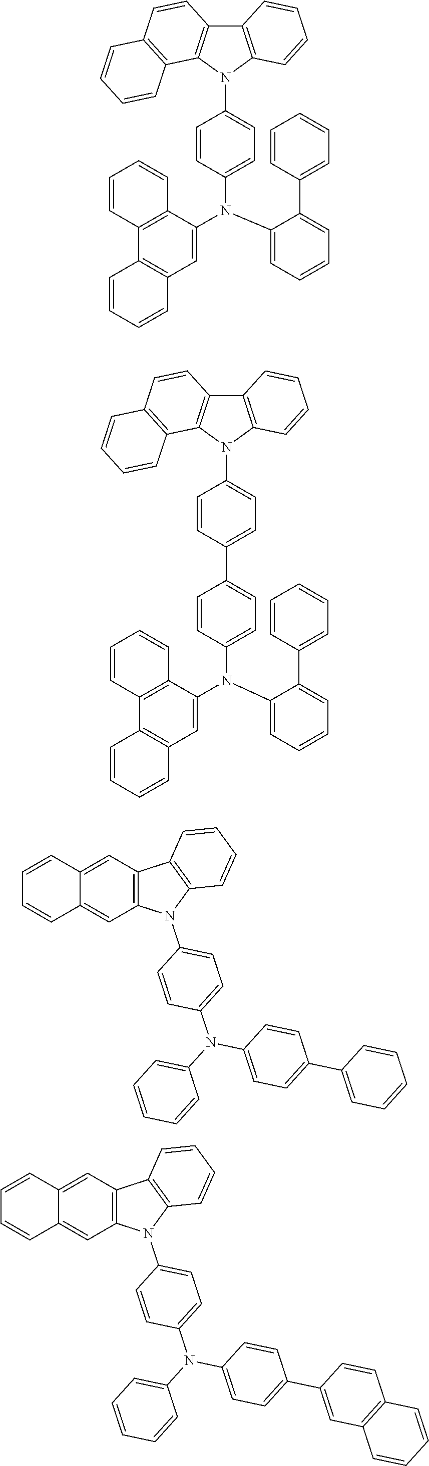

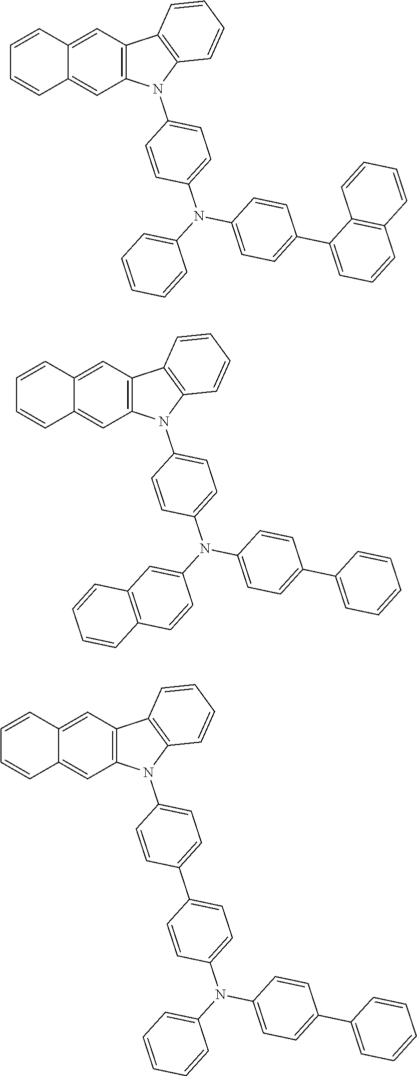

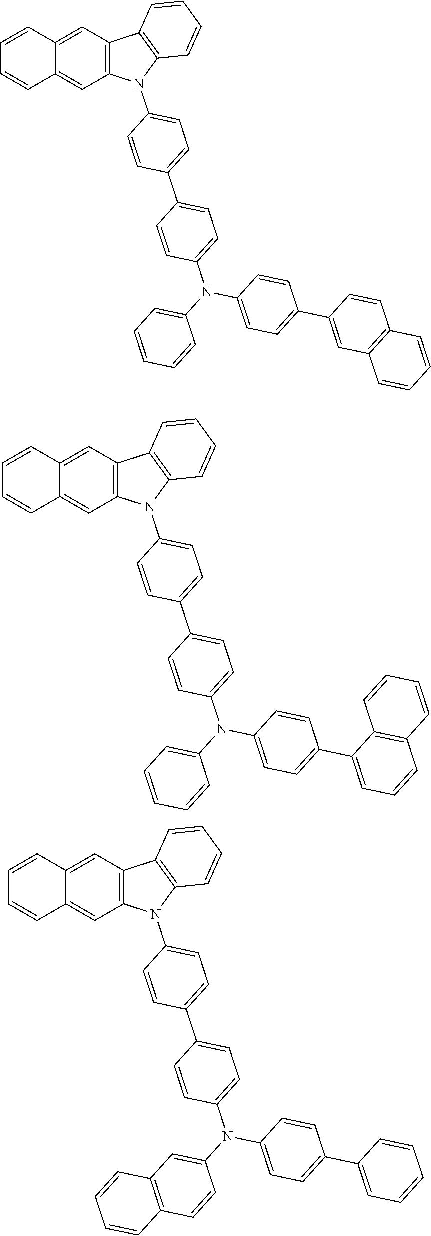

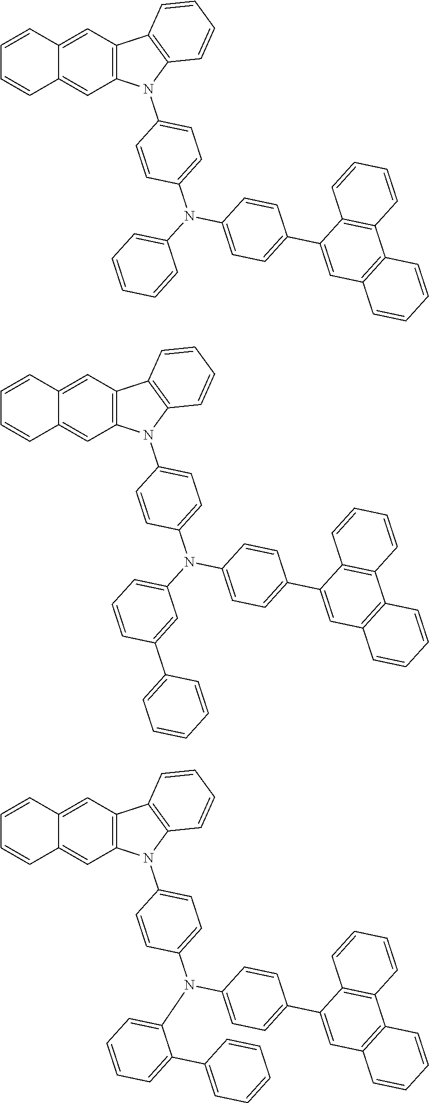

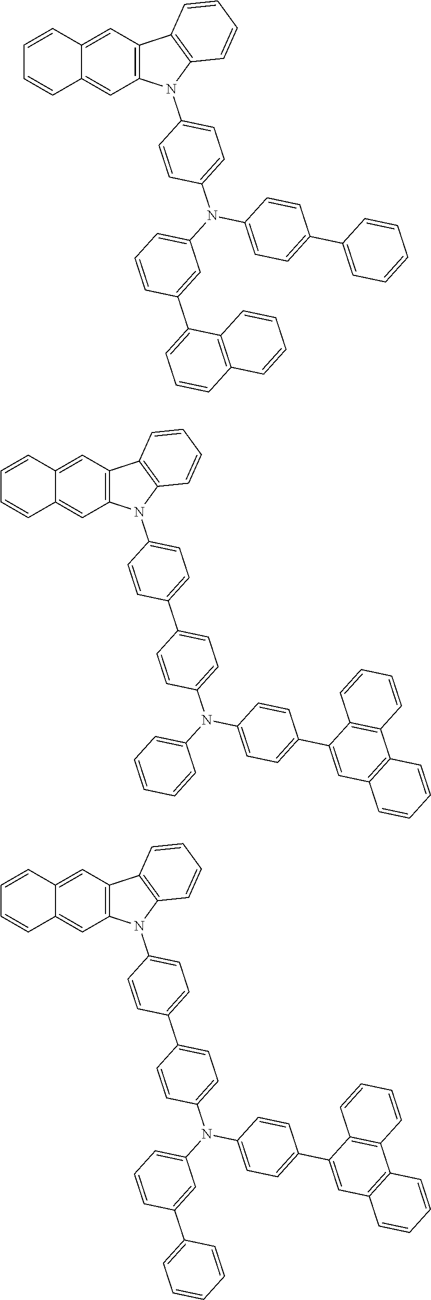 [SMILES: c1ccc(-c2ccc(N(c3ccc(-n4c5ccccc5c5cc6ccccc6cc54)cc3)c3cccc(-c4cccc5ccccc45)c3)cc2)cc1 ; c1ccc(N(c2ccc(-c3ccc(-n4c5ccccc5c5cc6ccccc6cc54)cc3)cc2)c2ccc(-c3cc4ccccc4c4ccccc34)cc2)cc1 ; c1ccc(-c2cccc(N(c3ccc(-c4ccc(-n5c6ccccc6c6cc7ccccc7cc65)cc4)cc3)c3ccc(-c4cc5ccccc5c5ccccc45)cc3)c2)cc1]

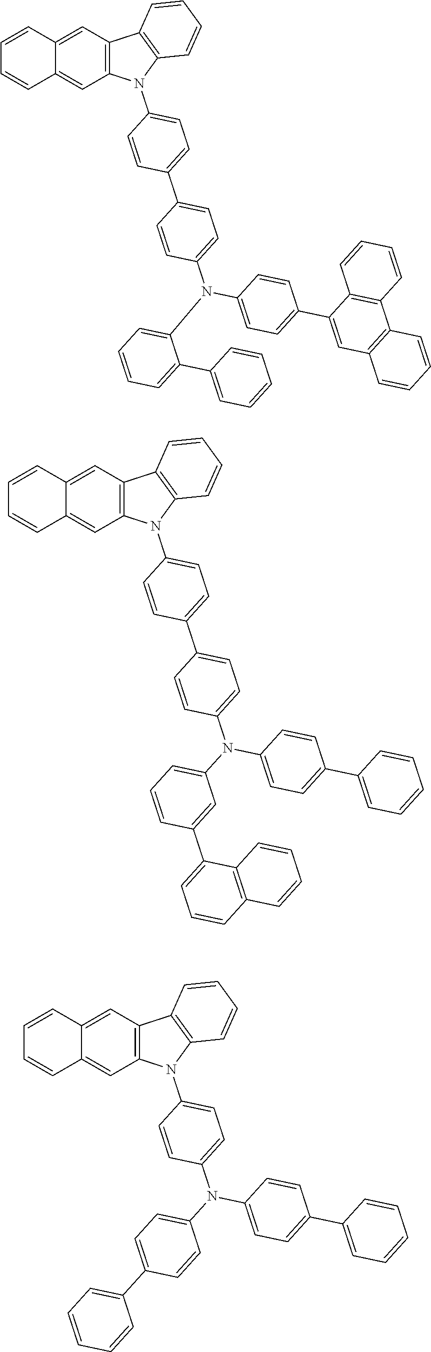

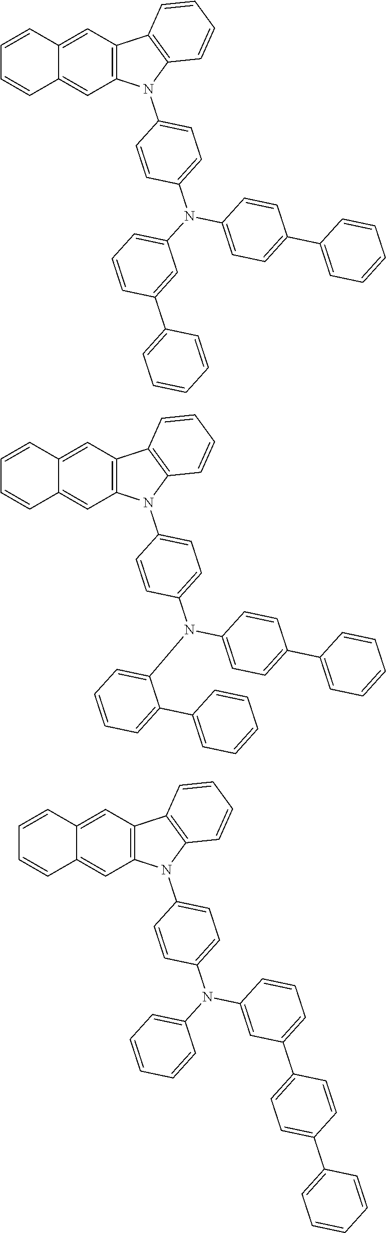

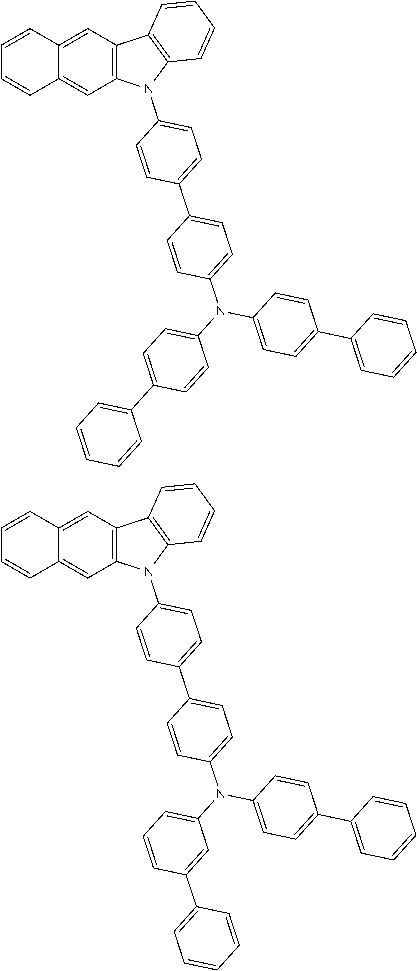 [SMILES: c1ccc(-c2ccc(N(c3ccc(-c4ccccc4)cc3)c3ccc(-c4ccc(-n5c6ccccc6c6cc7ccccc7cc65)cc4)cc3)cc2)cc1 ; c1ccc(-c2ccc(N(c3ccc(-c4ccc(-n5c6ccccc6c6cc7ccccc7cc65)cc4)cc3)c3cccc(-c4ccccc4)c3)cc2)cc1]

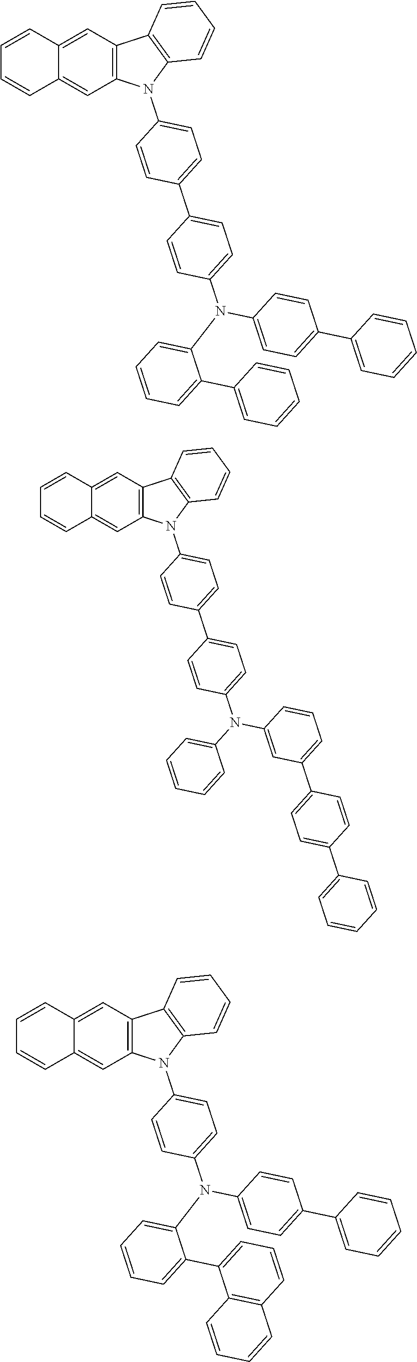

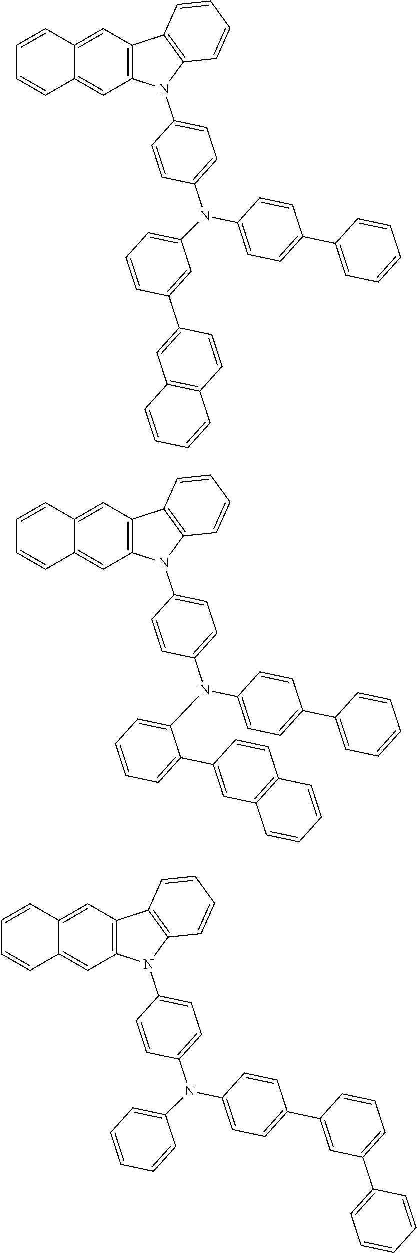

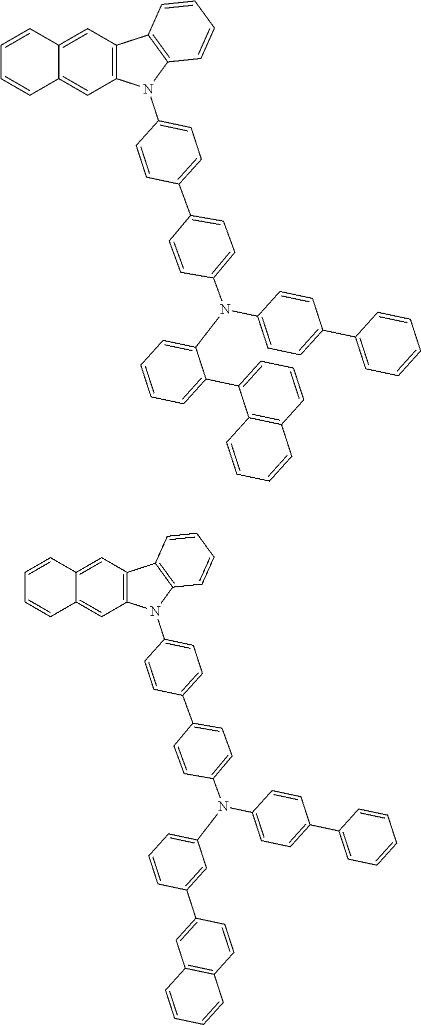 [SMILES: c1ccc(-c2ccc(N(c3ccc(-c4ccc(-n5c6ccccc6c6cc7ccccc7cc65)cc4)cc3)c3ccccc3-c3cccc4ccccc34)cc2)cc1 ; c1ccc(-c2ccc(N(c3ccc(-c4ccc(-n5c6ccccc6c6cc7ccccc7cc65)cc4)cc3)c3cccc(-c4ccc5ccccc5c4)c3)cc2)cc1]

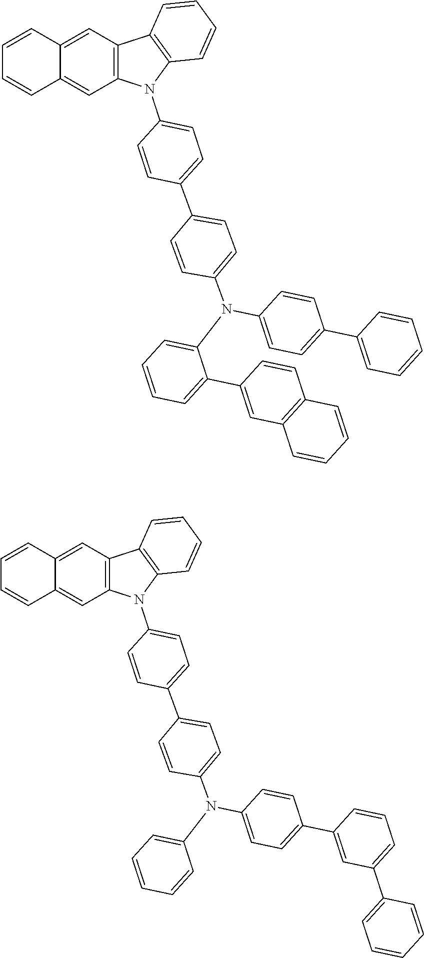 [SMILES: c1ccc(-c2ccc(N(c3ccc(-c4ccc(-n5c6ccccc6c6cc7ccccc7cc65)cc4)cc3)c3ccccc3-c3ccc4ccccc4c3)cc2)cc1 ; c1ccc(-c2cccc(-c3ccc(N(c4ccccc4)c4ccc(-c5ccc(-n6c7ccccc7c7cc8ccccc8cc76)cc5)cc4)cc3)c2)cc1]

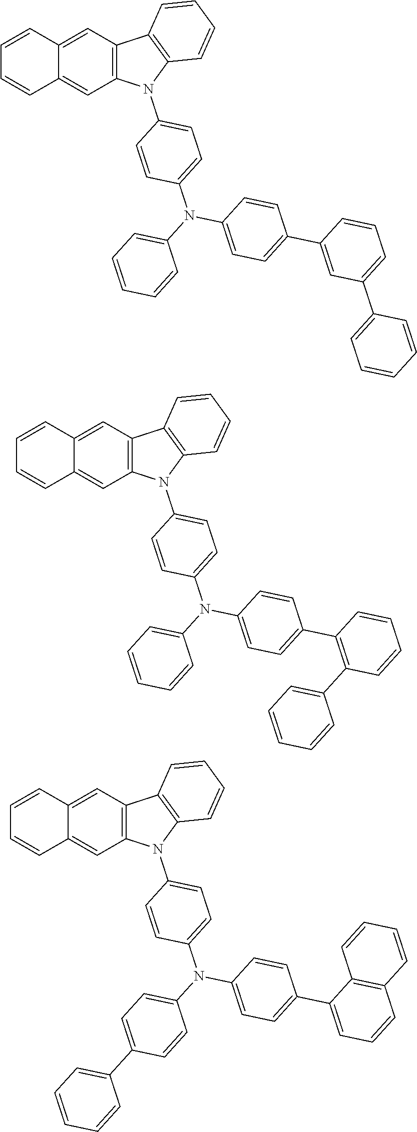 [SMILES: c1ccc(-c2cccc(-c3ccc(N(c4ccccc4)c4ccc(-n5c6ccccc6c6cc7ccccc7cc65)cc4)cc3)c2)cc1 ; c1ccc(-c2ccccc2-c2ccc(N(c3ccccc3)c3ccc(-n4c5ccccc5c5cc6ccccc6cc54)cc3)cc2)cc1 ; c1ccc(-c2ccc(N(c3ccc(-c4cccc5ccccc45)cc3)c3ccc(-n4c5ccccc5c5cc6ccccc6cc54)cc3)cc2)cc1]

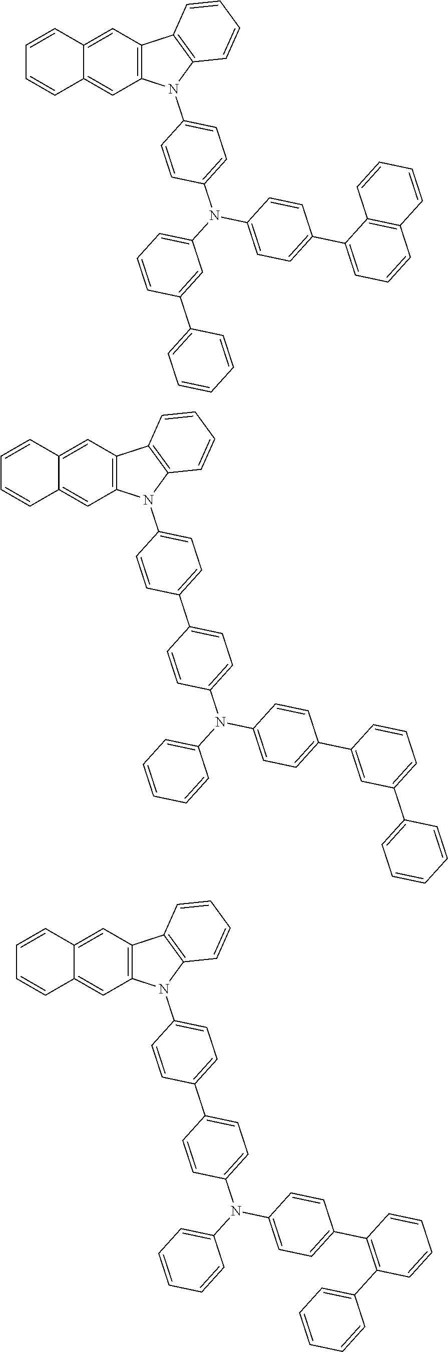

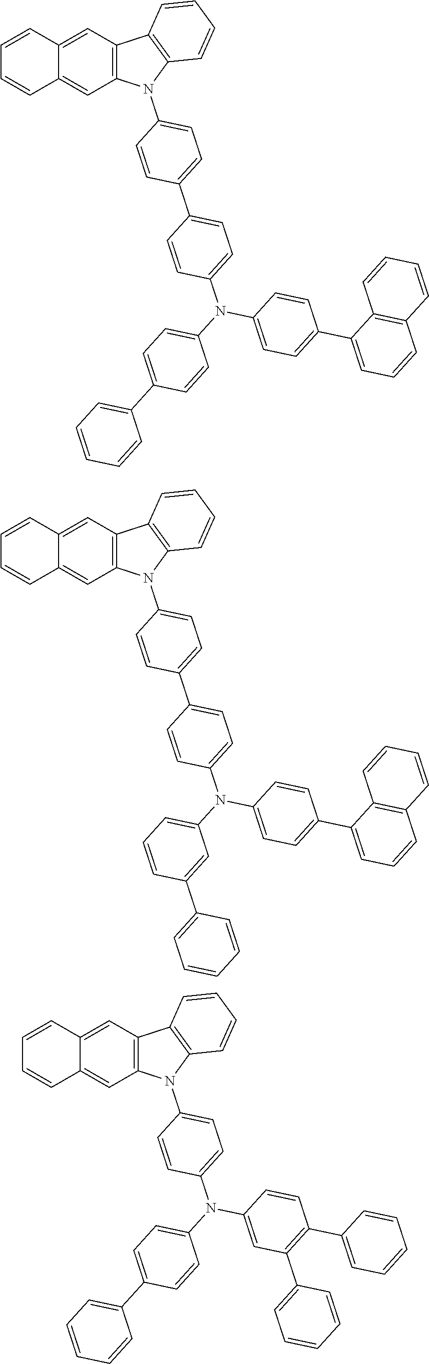

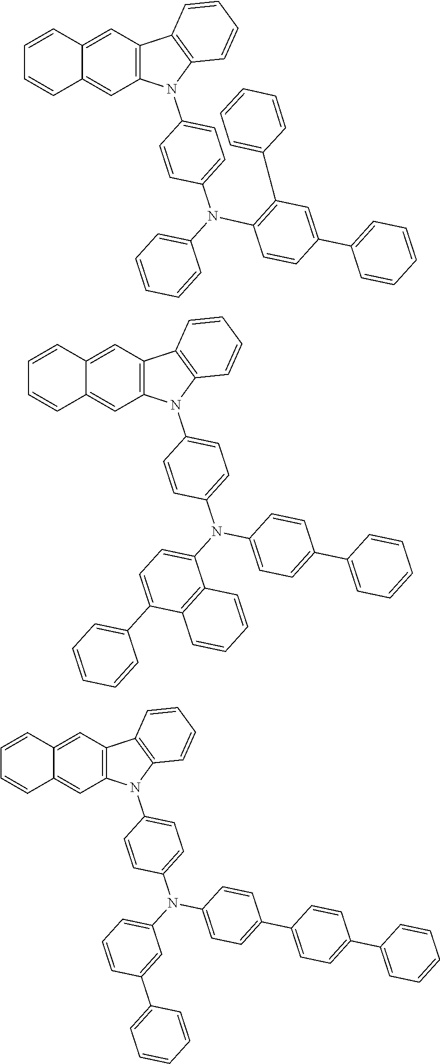

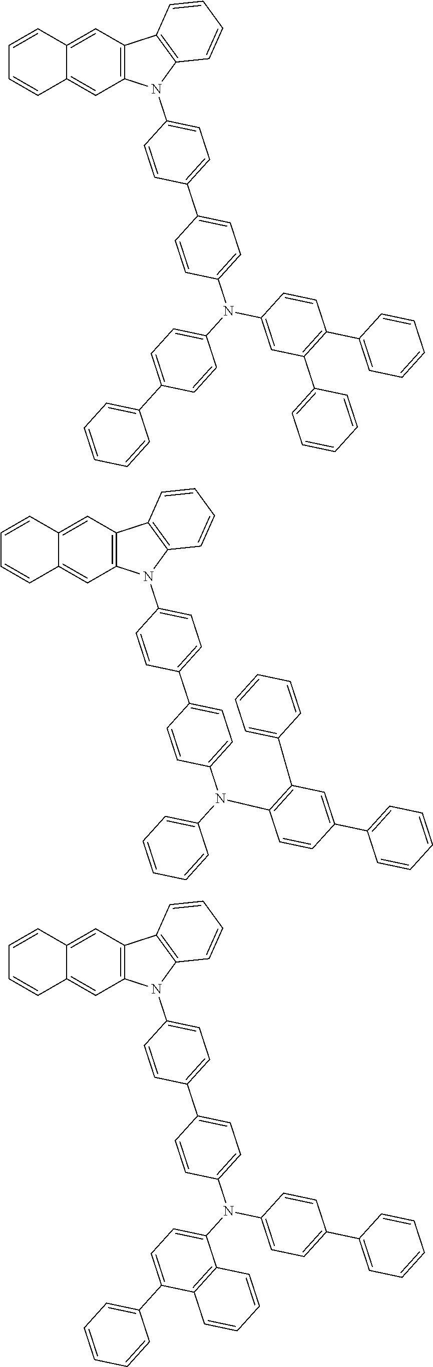

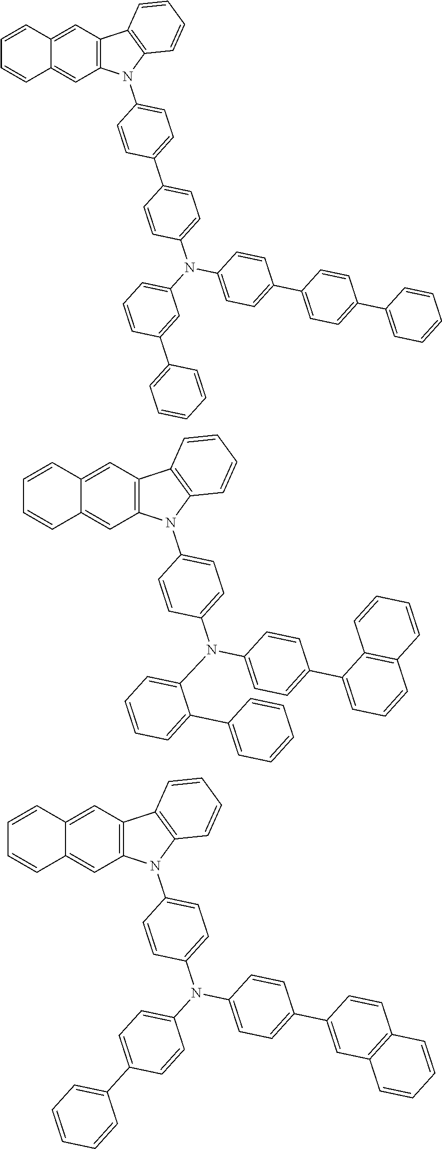

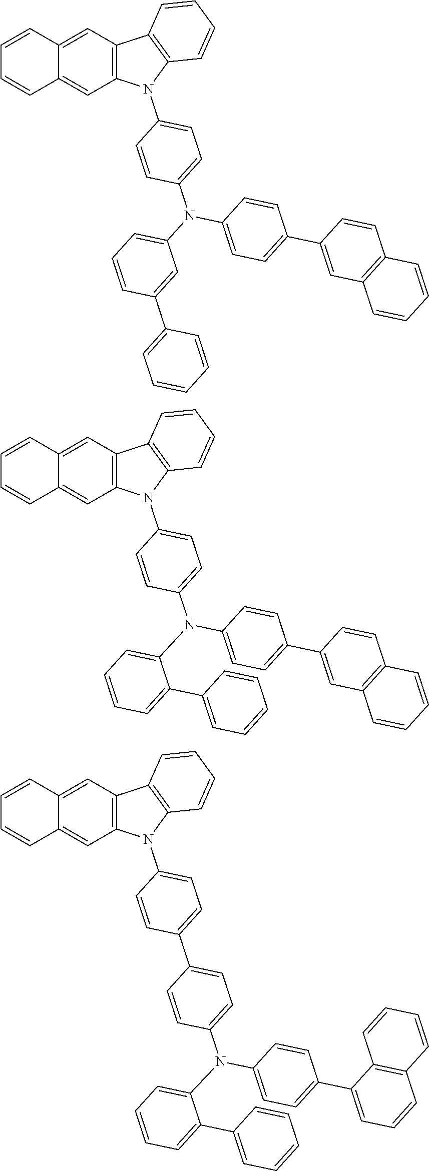

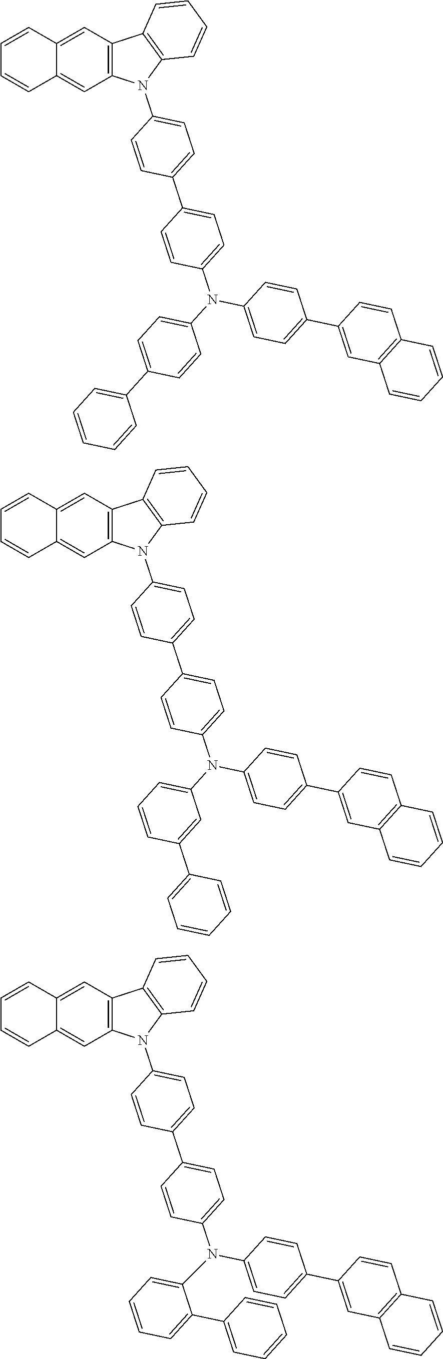

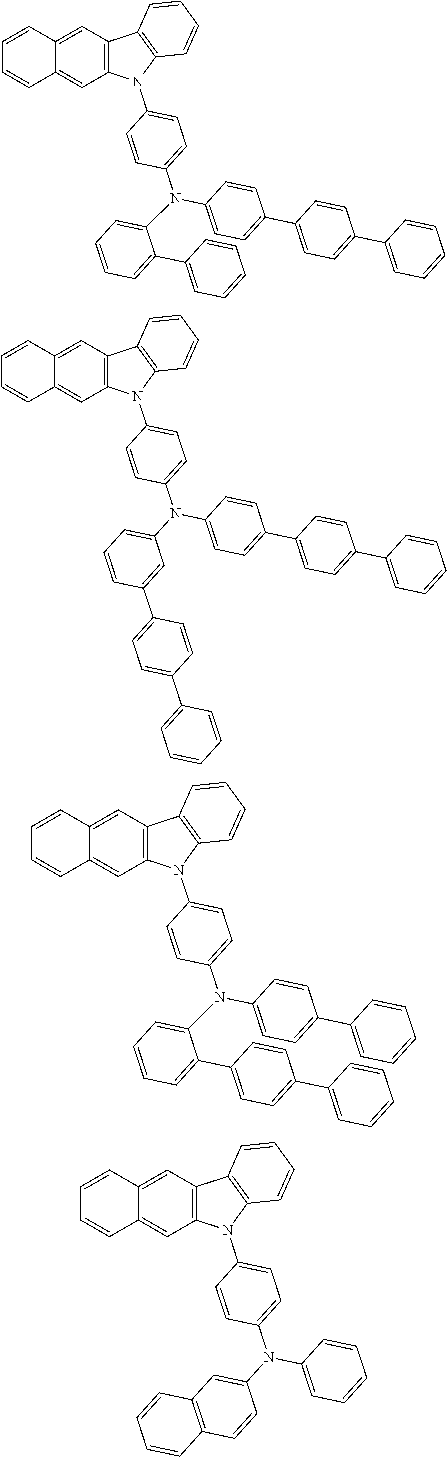

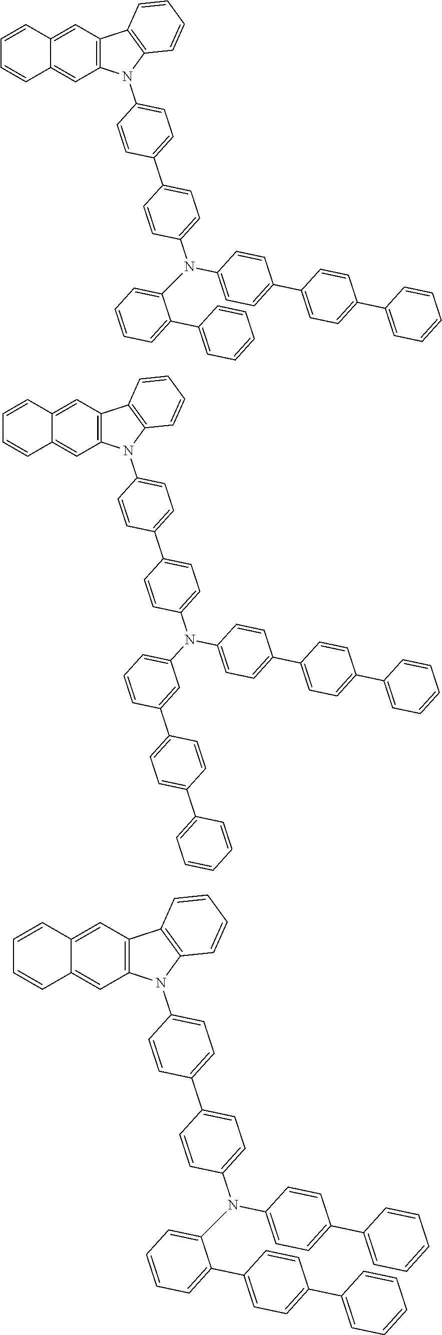

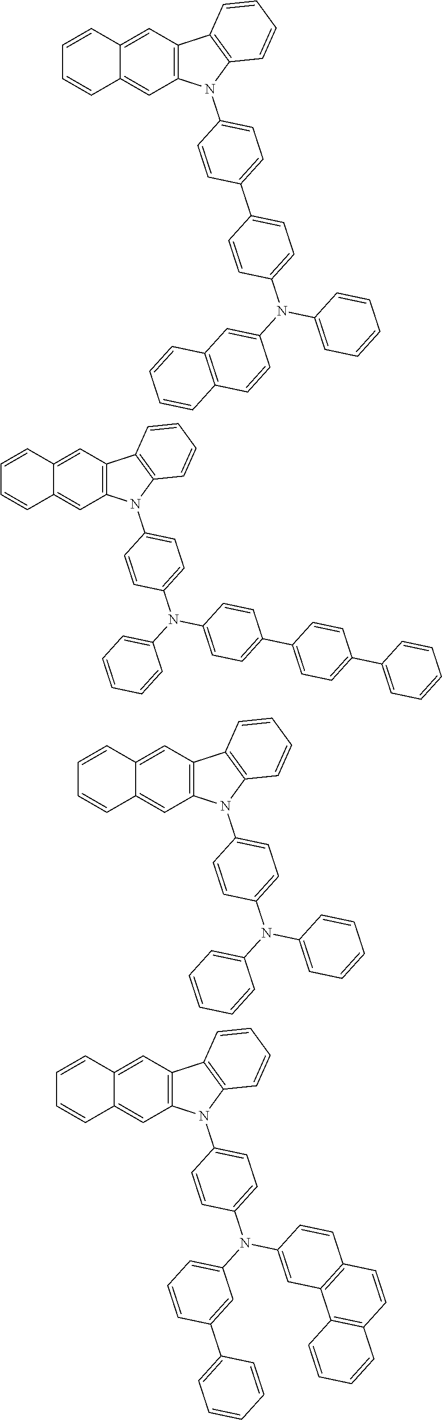

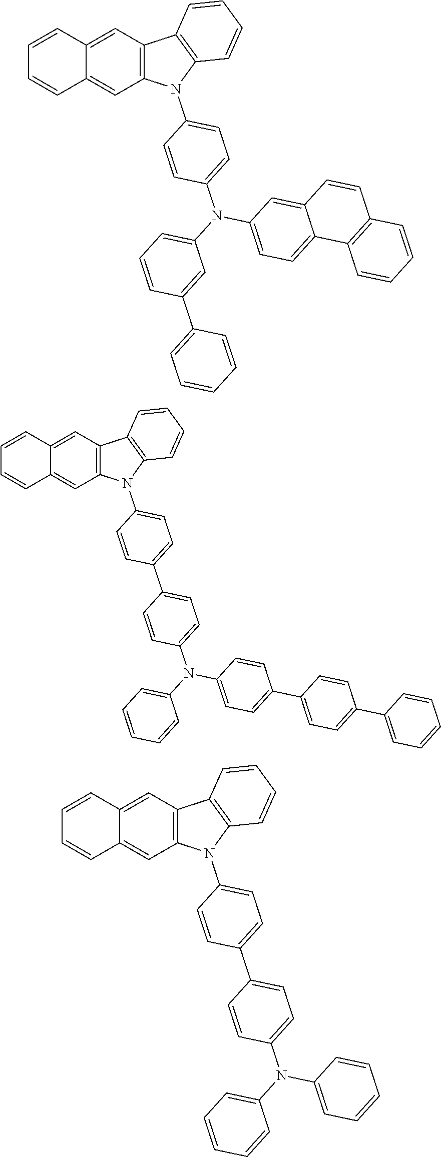

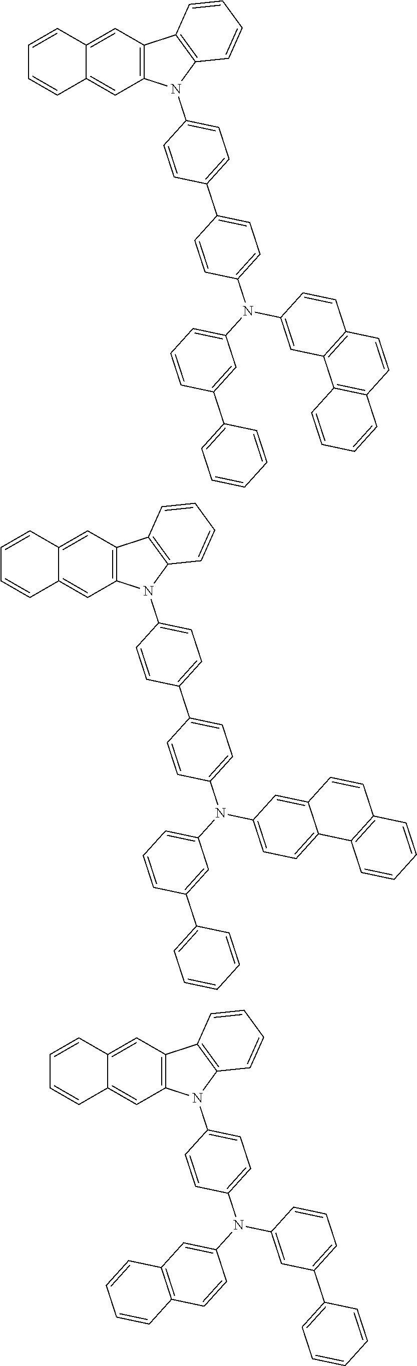

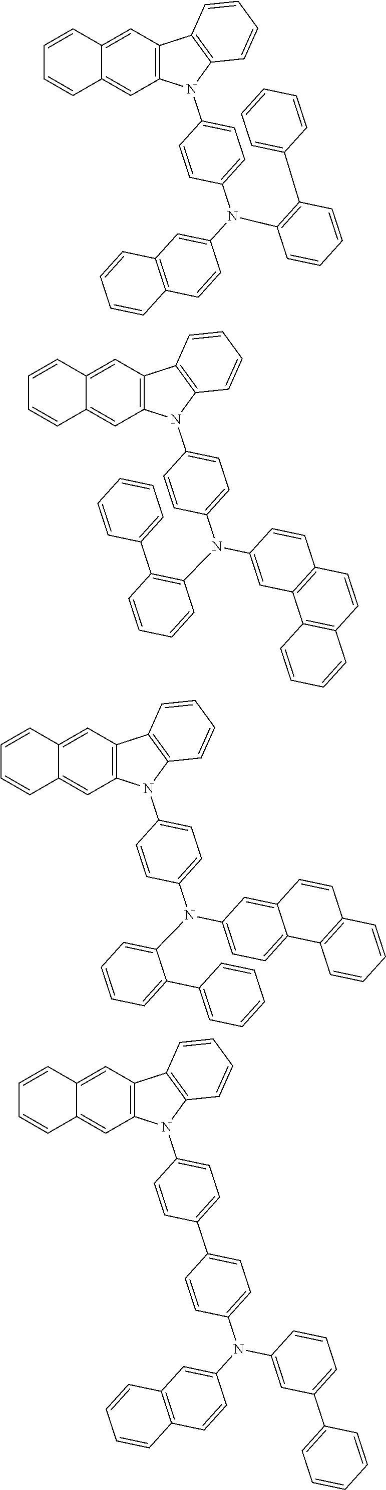

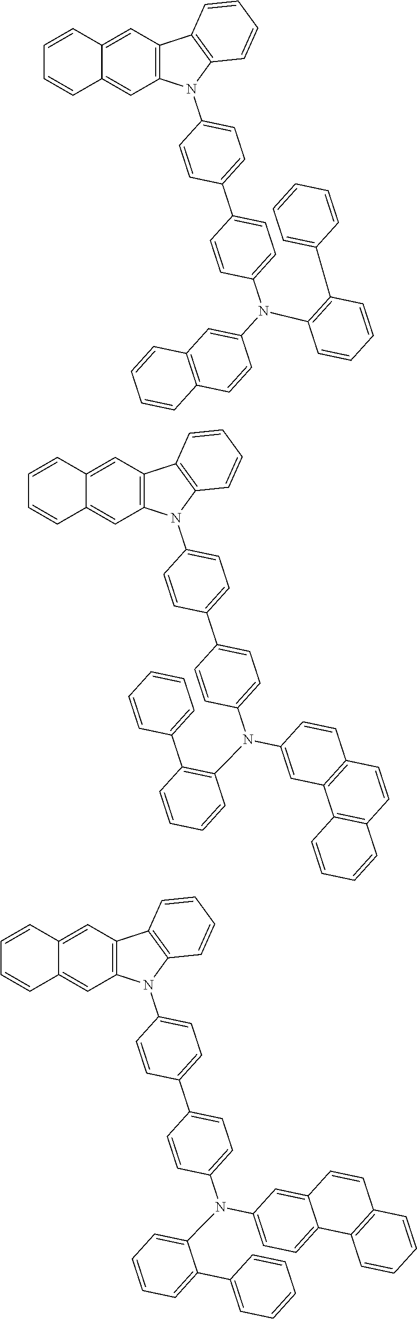

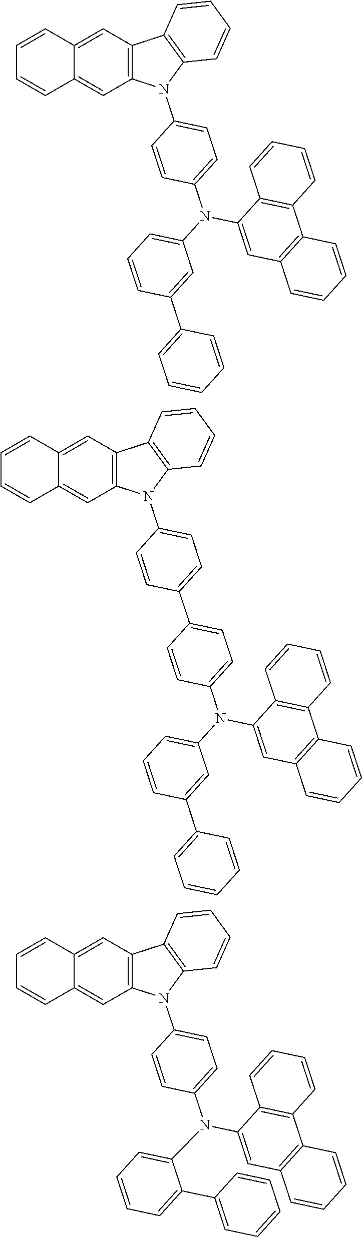

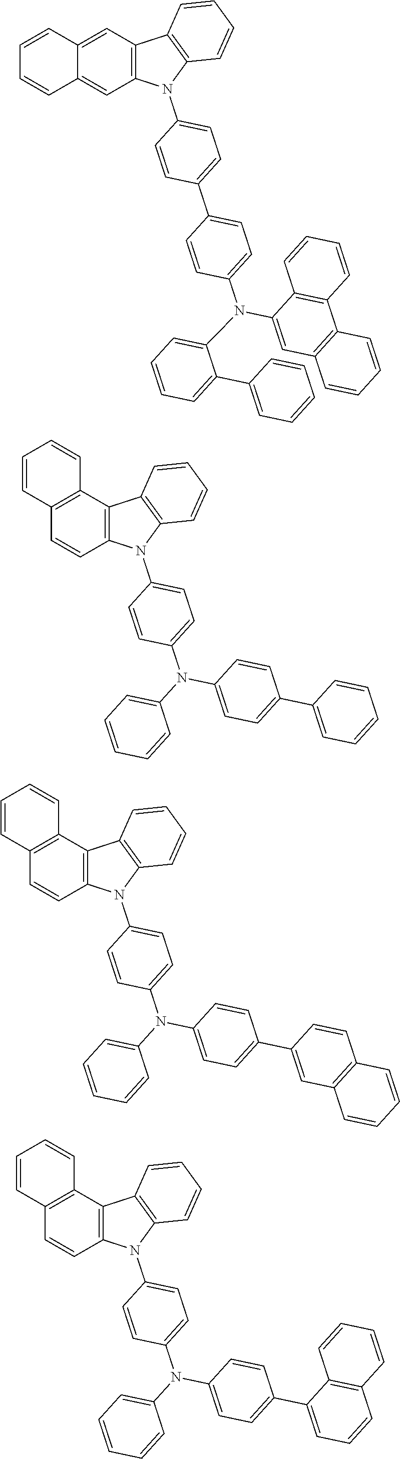

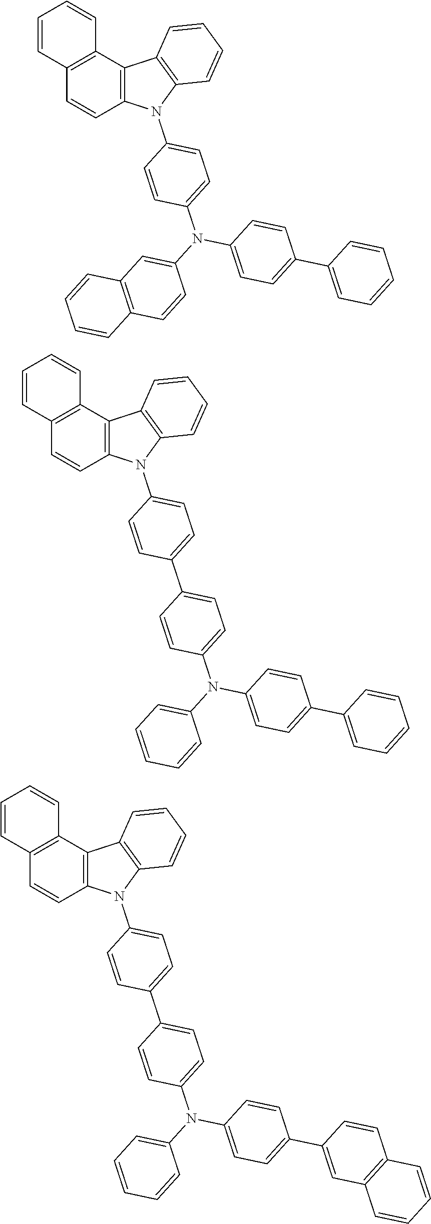

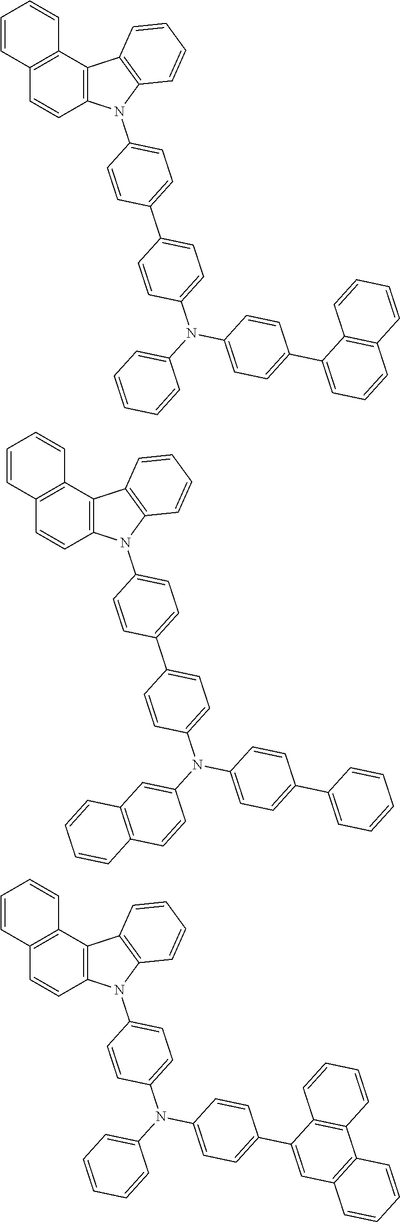

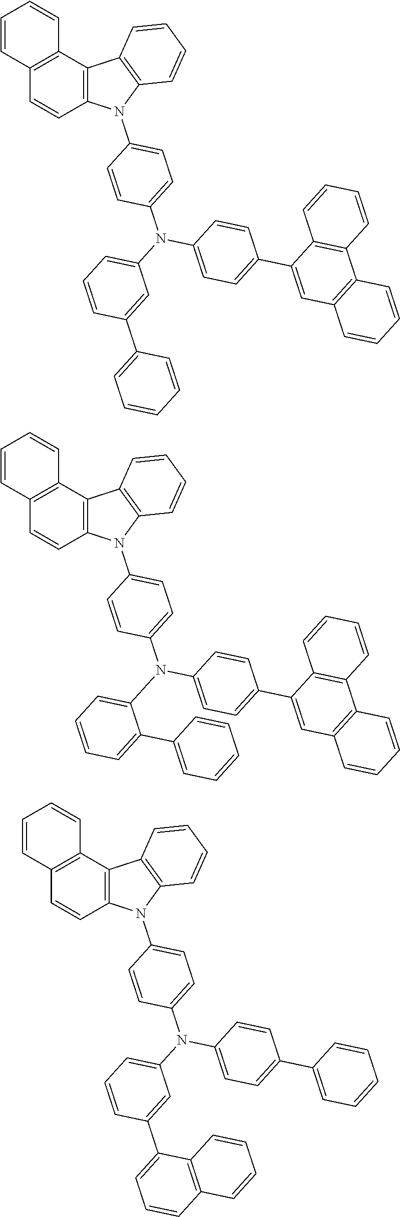

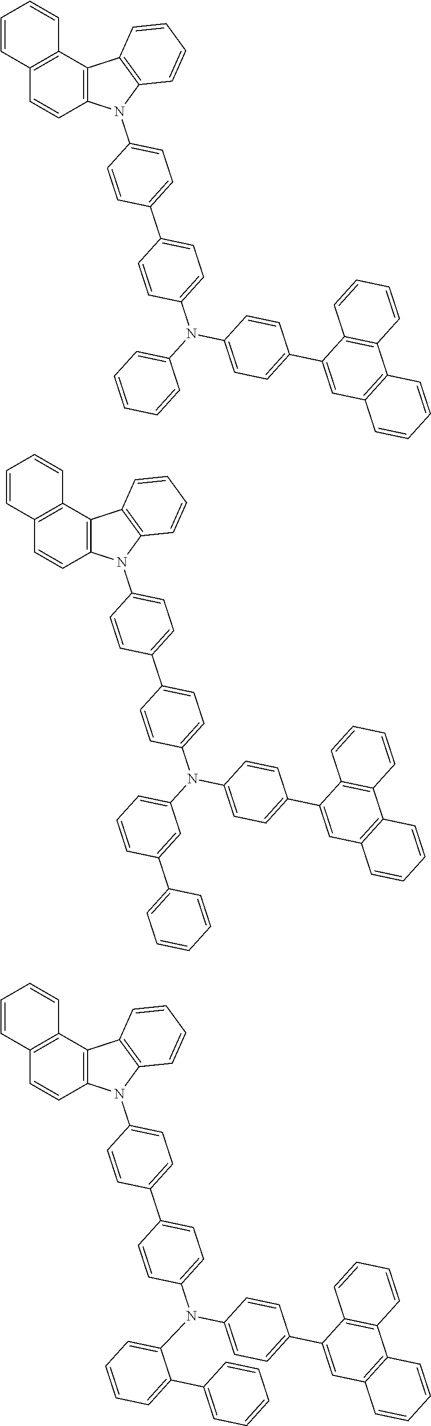 [SMILES: c1ccc(N(c2ccc(-c3ccc(-n4c5ccccc5c5c6ccccc6ccc54)cc3)cc2)c2ccc(-c3cc4ccccc4c4ccccc34)cc2)cc1 ; c1ccc(-c2cccc(N(c3ccc(-c4ccc(-n5c6ccccc6c6c7ccccc7ccc65)cc4)cc3)c3ccc(-c4cc5ccccc5c5ccccc45)cc3)c2)cc1 ; c1ccc(-c2ccccc2N(c2ccc(-c3ccc(-n4c5ccccc5c5c6ccccc6ccc54)cc3)cc2)c2ccc(-c3cc4ccccc4c4ccccc34)cc2)cc1]

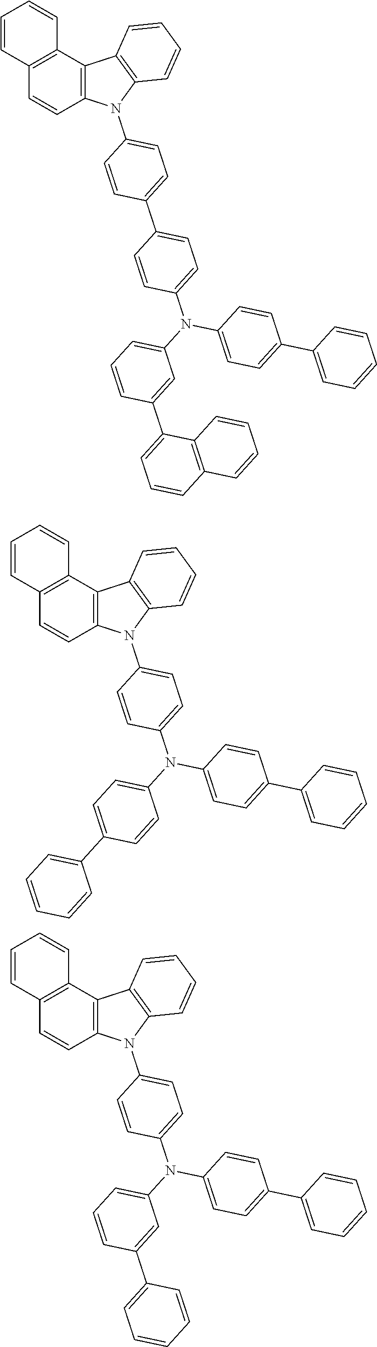

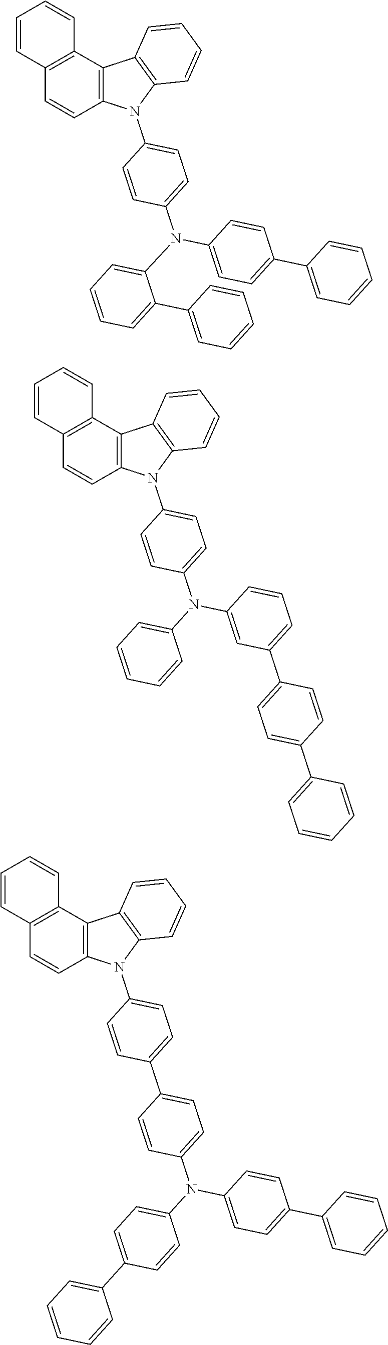 [SMILES: c1ccc(-c2ccc(N(c3ccc(-n4c5ccccc5c5c6ccccc6ccc54)cc3)c3ccccc3-c3ccccc3)cc2)cc1 ; c1ccc(-c2ccc(-c3cccc(N(c4ccccc4)c4ccc(-n5c6ccccc6c6c7ccccc7ccc65)cc4)c3)cc2)cc1 ; c1ccc(-c2ccc(N(c3ccc(-c4ccccc4)cc3)c3ccc(-c4ccc(-n5c6ccccc6c6c7ccccc7ccc65)cc4)cc3)cc2)cc1]

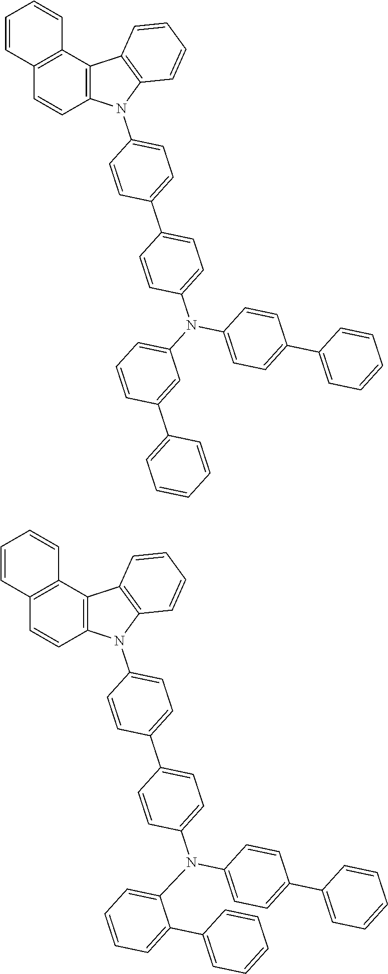 [SMILES: c1ccc(-c2ccc(N(c3ccc(-c4ccc(-n5c6ccccc6c6c7ccccc7ccc65)cc4)cc3)c3cccc(-c4ccccc4)c3)cc2)cc1 ; c1ccc(-c2ccc(N(c3ccc(-c4ccc(-n5c6ccccc6c6c7ccccc7ccc65)cc4)cc3)c3ccccc3-c3ccccc3)cc2)cc1]

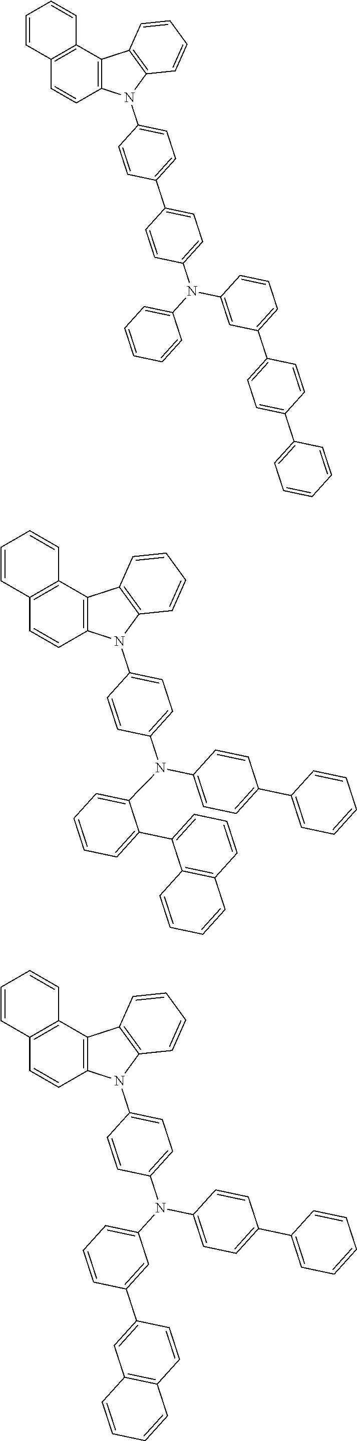 [SMILES: c1ccc(-c2ccc(-c3cccc(N(c4ccccc4)c4ccc(-c5ccc(-n6c7ccccc7c7c8ccccc8ccc76)cc5)cc4)c3)cc2)cc1 ; c1ccc(-c2ccc(N(c3ccc(-n4c5ccccc5c5c6ccccc6ccc54)cc3)c3ccccc3-c3cccc4ccccc34)cc2)cc1 ; c1ccc(-c2ccc(N(c3ccc(-n4c5ccccc5c5c6ccccc6ccc54)cc3)c3cccc(-c4ccc5ccccc5c4)c3)cc2)cc1]

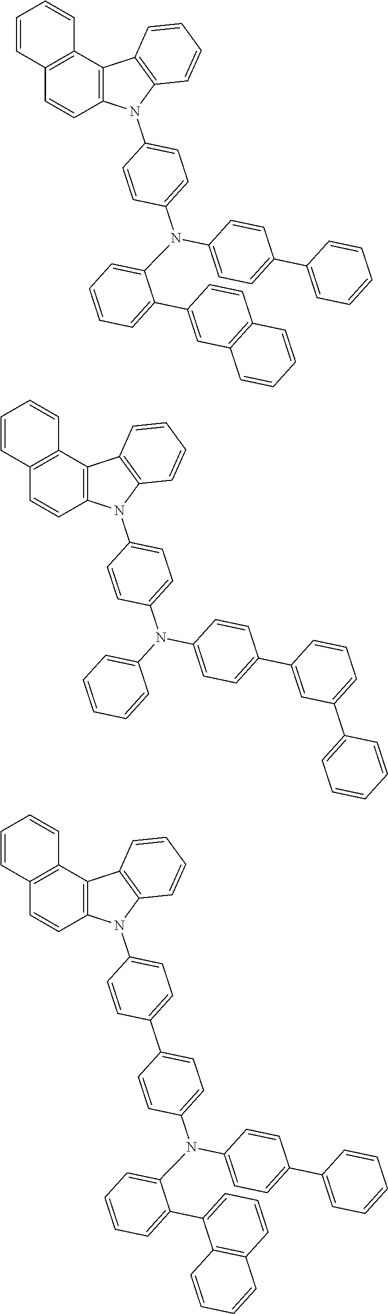 [SMILES: c1ccc(-c2ccc(N(c3ccc(-n4c5ccccc5c5c6ccccc6ccc54)cc3)c3ccccc3-c3ccc4ccccc4c3)cc2)cc1 ; c1ccc(-c2cccc(-c3ccc(N(c4ccccc4)c4ccc(-n5c6ccccc6c6c7ccccc7ccc65)cc4)cc3)c2)cc1 ; c1ccc(-c2ccc(N(c3ccc(-c4ccc(-n5c6ccccc6c6c7ccccc7ccc65)cc4)cc3)c3ccccc3-c3cccc4ccccc34)cc2)cc1]

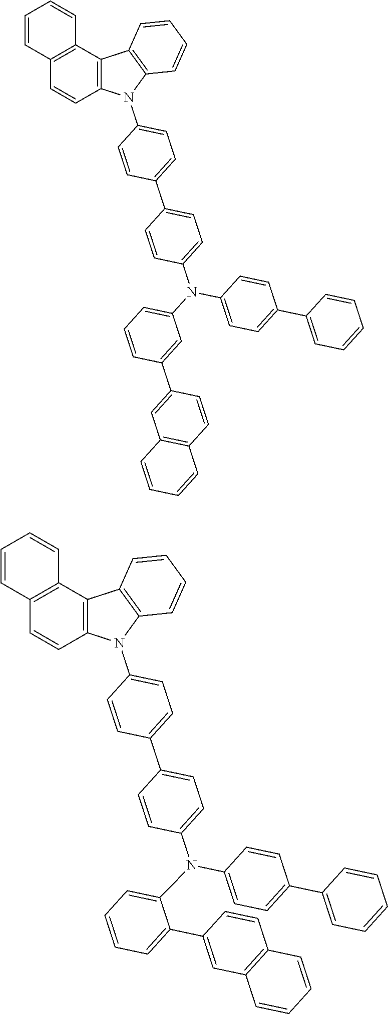 [SMILES: c1ccc(-c2ccc(N(c3ccc(-c4ccc(-n5c6ccccc6c6c7ccccc7ccc65)cc4)cc3)c3cccc(-c4ccc5ccccc5c4)c3)cc2)cc1 ; c1ccc(-c2ccc(N(c3ccc(-c4ccc(-n5c6ccccc6c6c7ccccc7ccc65)cc4)cc3)c3ccccc3-c3ccc4ccccc4c3)cc2)cc1]

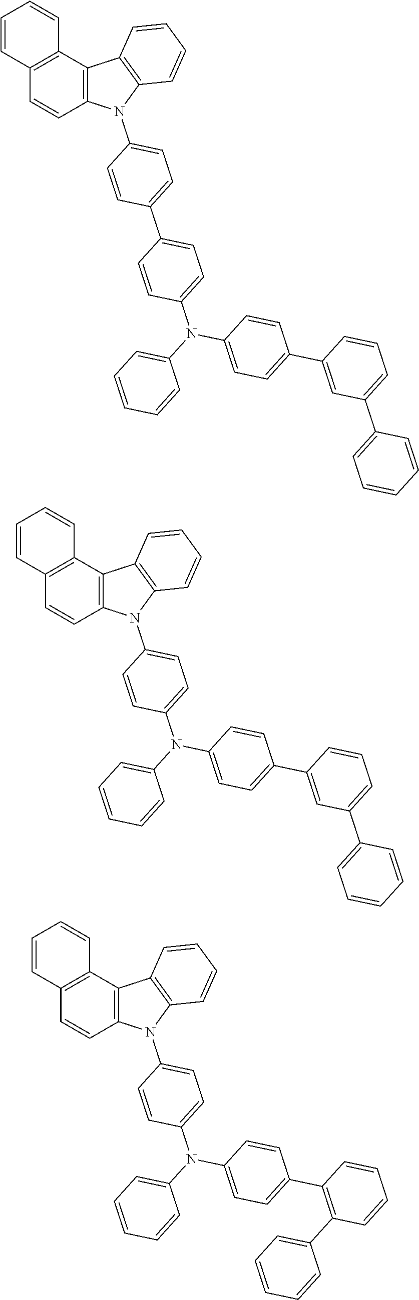 [SMILES: c1ccc(-c2cccc(-c3ccc(N(c4ccccc4)c4ccc(-c5ccc(-n6c7ccccc7c7c8ccccc8ccc76)cc5)cc4)cc3)c2)cc1 ; c1ccc(-c2cccc(-c3ccc(N(c4ccccc4)c4ccc(-n5c6ccccc6c6c7ccccc7ccc65)cc4)cc3)c2)cc1 ; c1ccc(-c2ccccc2-c2ccc(N(c3ccccc3)c3ccc(-n4c5ccccc5c5c6ccccc6ccc54)cc3)cc2)cc1]

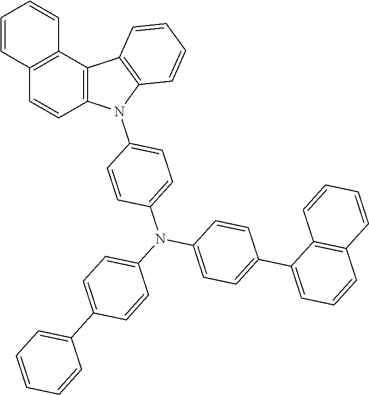 [SMILES: c1ccc(-c2ccc(N(c3ccc(-c4cccc5ccccc45)cc3)c3ccc(-n4c5ccccc5c5c6ccccc6ccc54)cc3)cc2)cc1]

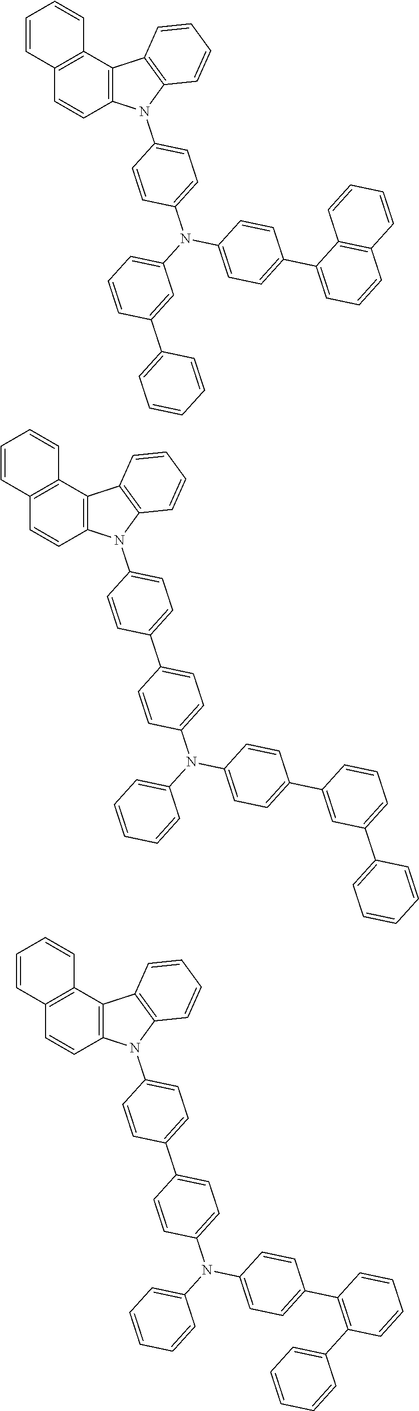

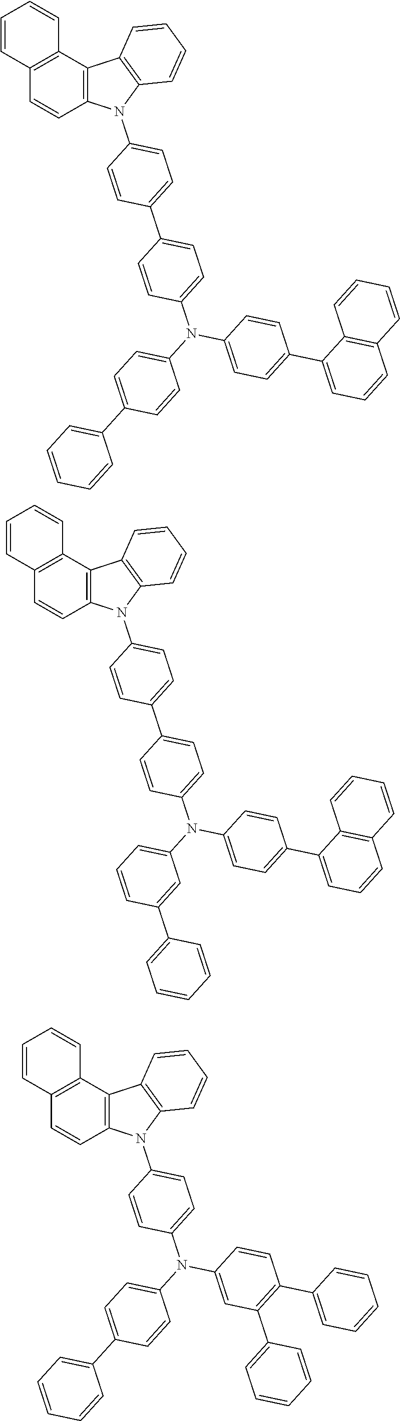

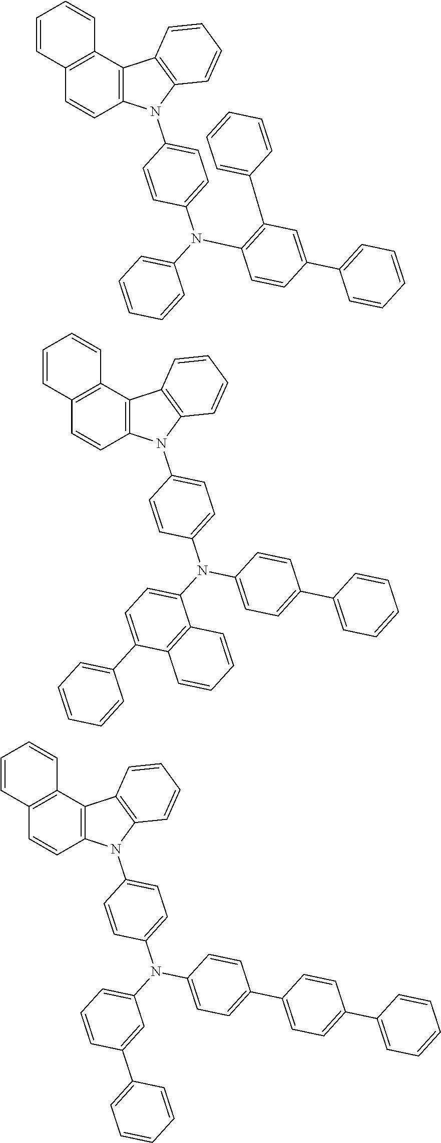

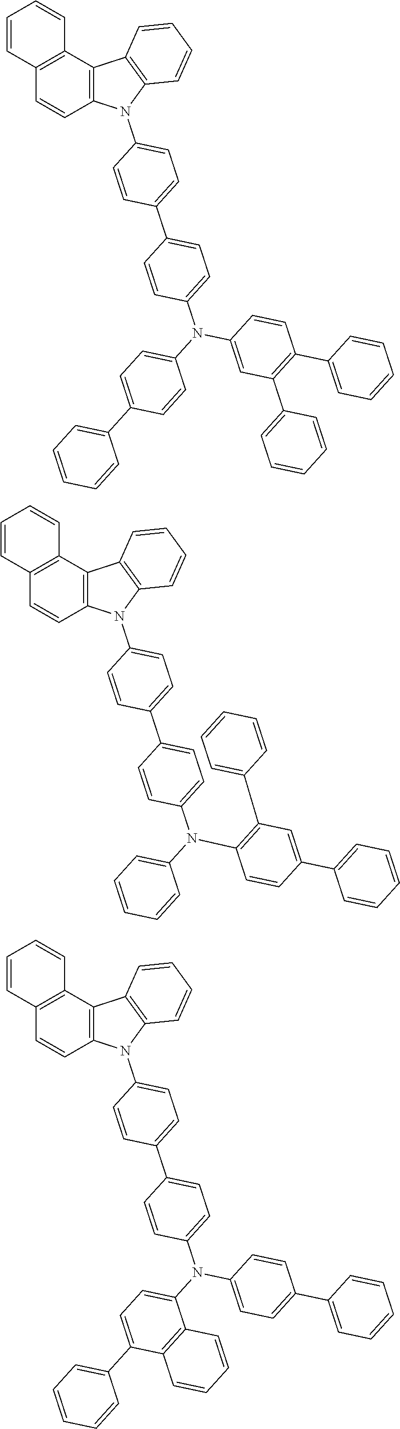

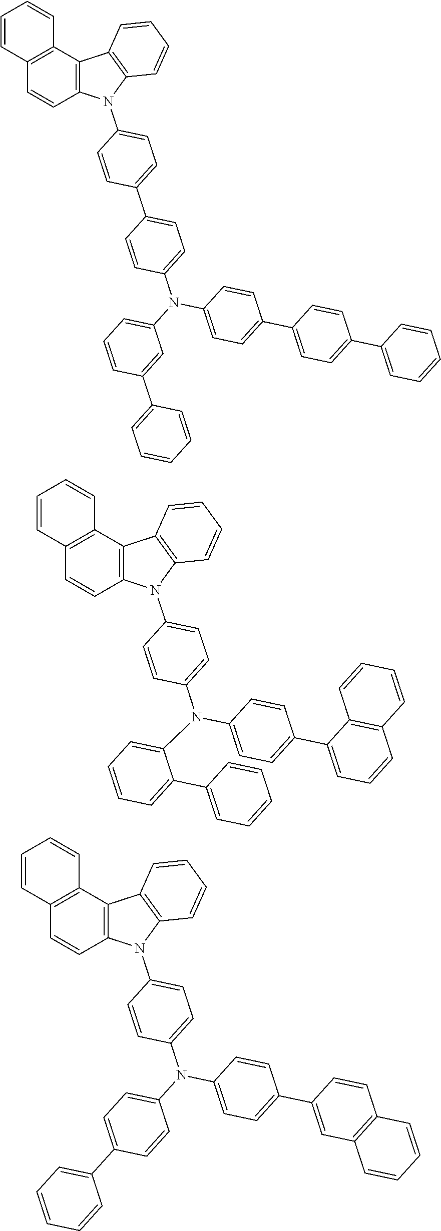

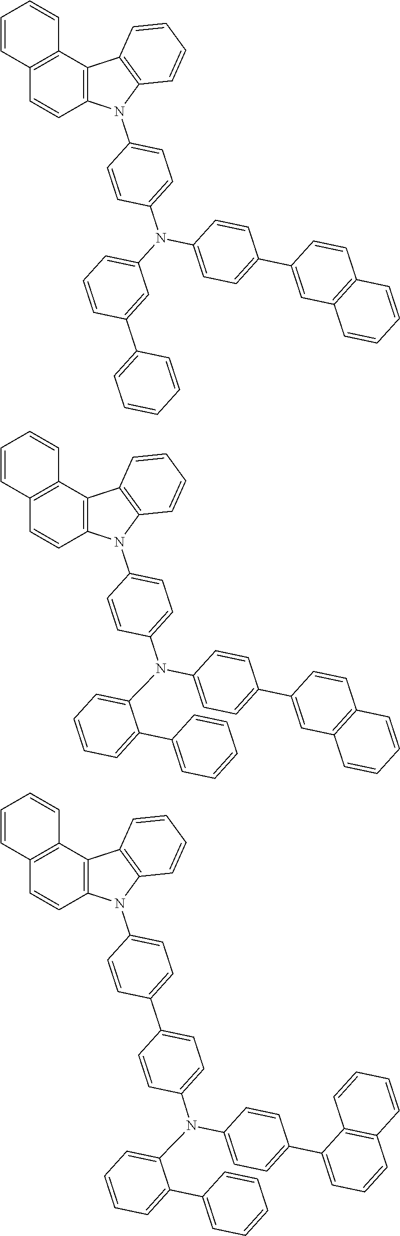

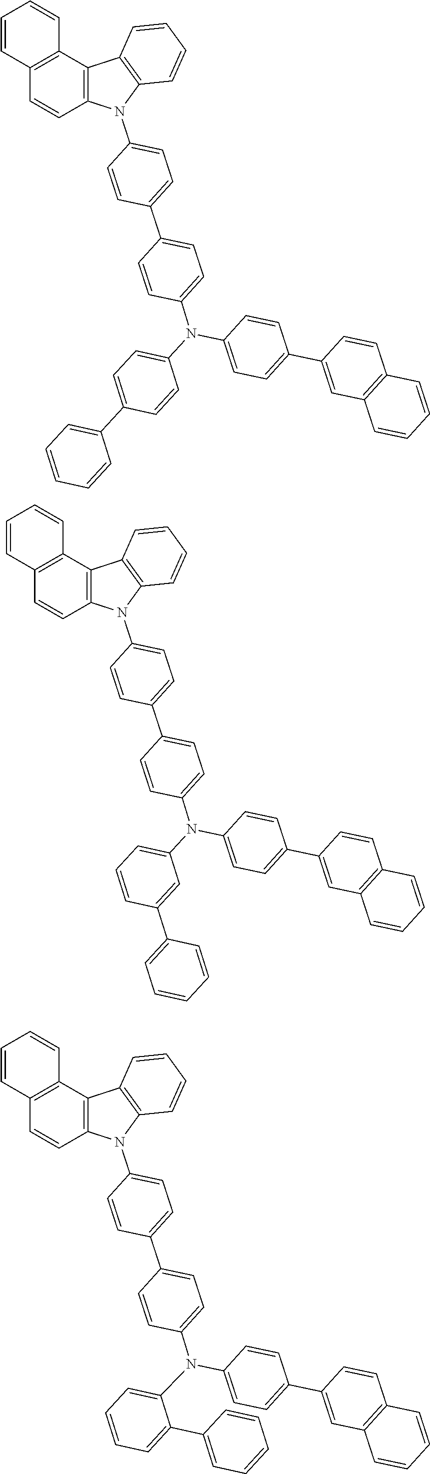

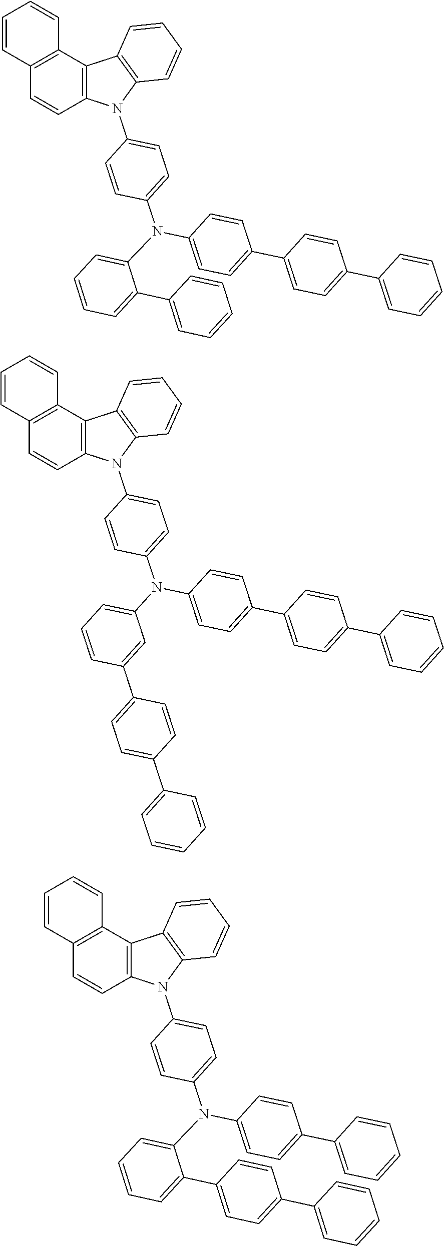 [SMILES: c1ccc(-c2ccc(-c3ccc(N(c4ccc(-n5c6ccccc6c6c7ccccc7ccc65)cc4)c4ccccc4-c4ccccc4)cc3)cc2)cc1 ; c1ccc(-c2ccc(-c3ccc(N(c4ccc(-n5c6ccccc6c6c7ccccc7ccc65)cc4)c4cccc(-c5ccc(-c6ccccc6)cc5)c4)cc3)cc2)cc1 ; c1ccc(-c2ccc(-c3ccccc3N(c3ccc(-c4ccccc4)cc3)c3ccc(-n4c5ccccc5c5c6ccccc6ccc54)cc3)cc2)cc1]

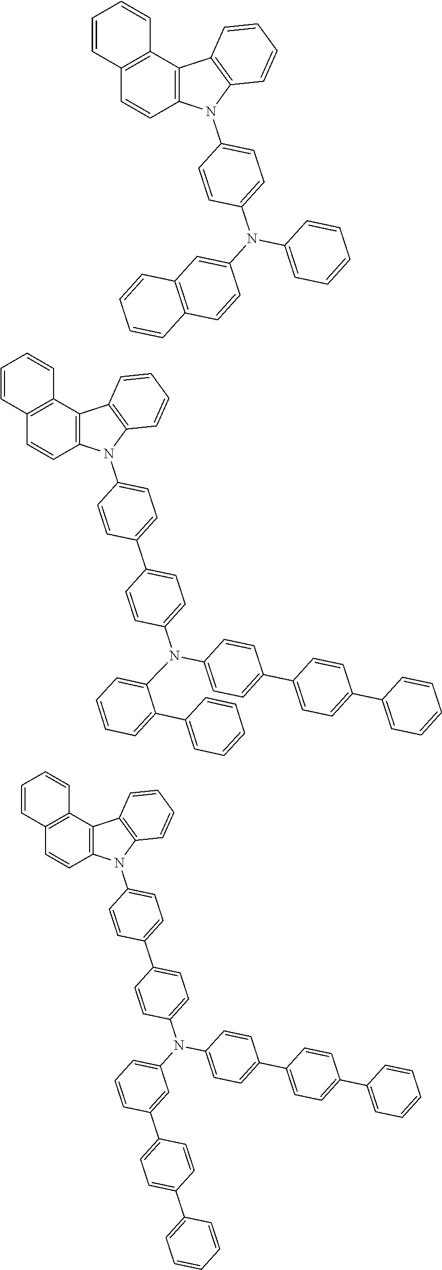

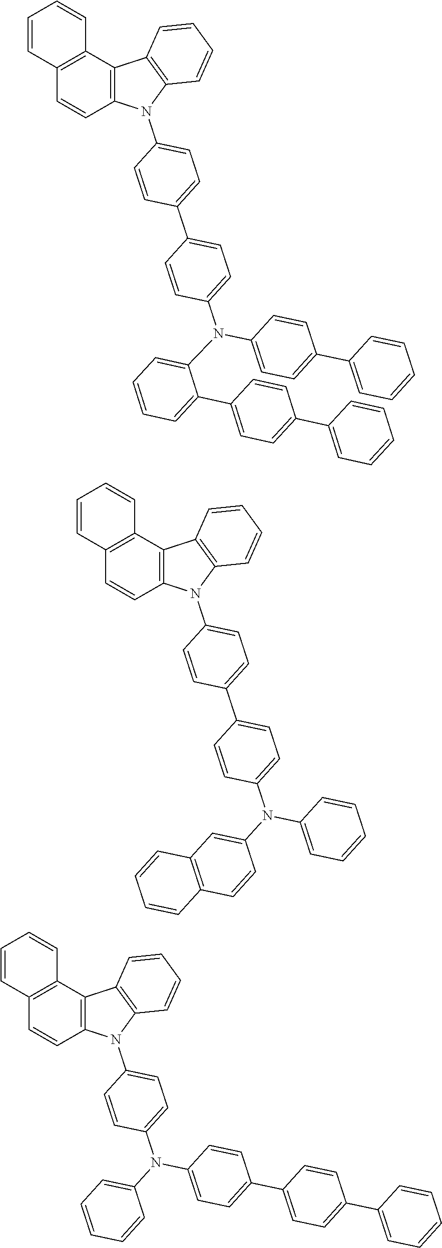

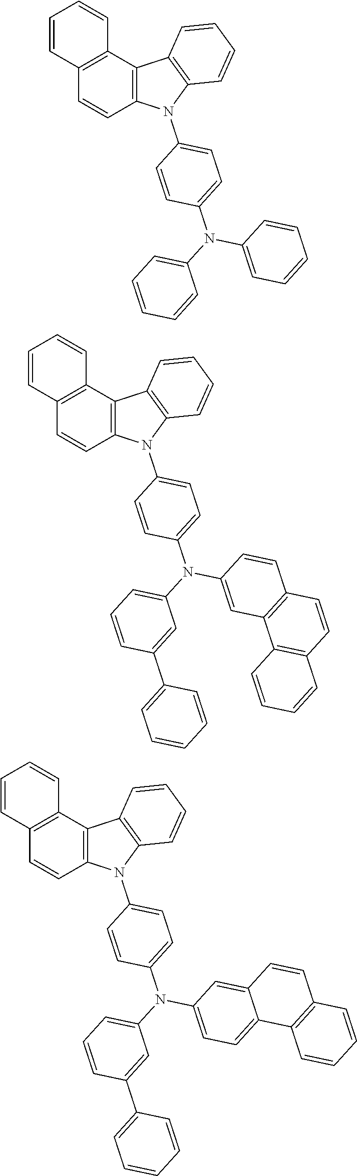

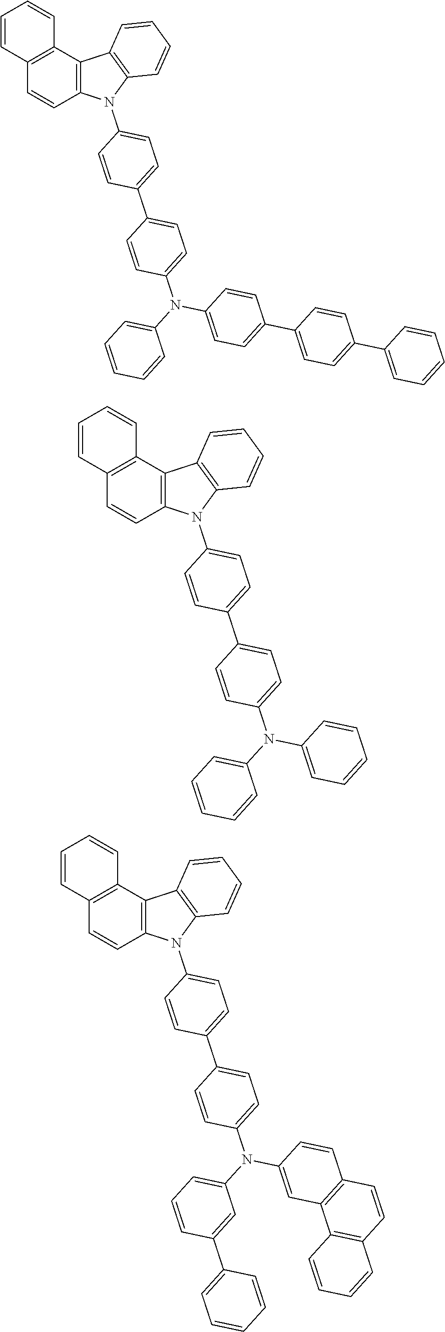

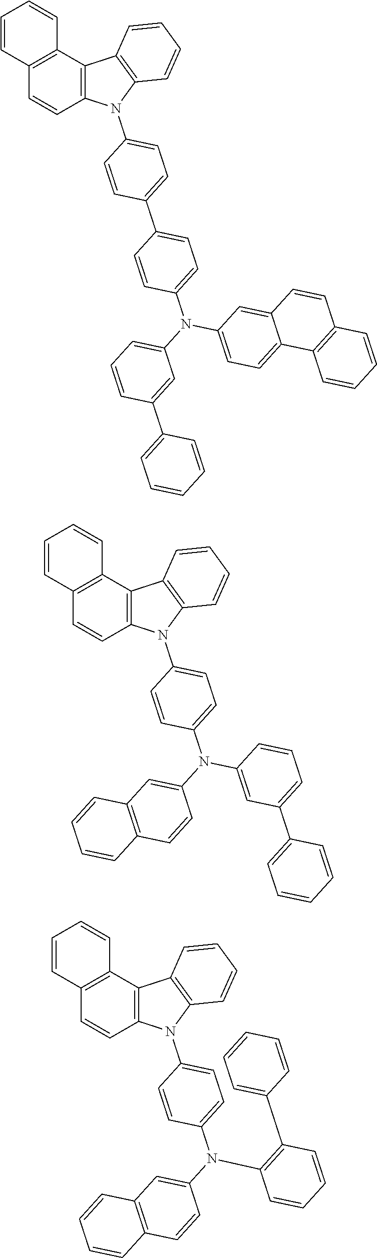 [SMILES: c1ccc(-c2cccc(N(c3ccc(-c4ccc(-n5c6ccccc6c6c7ccccc7ccc65)cc4)cc3)c3ccc4c(ccc5ccccc54)c3)c2)cc1 ; c1ccc(-c2cccc(N(c3ccc(-n4c5ccccc5c5c6ccccc6ccc54)cc3)c3ccc4ccccc4c3)c2)cc1 ; c1ccc(-c2ccccc2N(c2ccc(-n3c4ccccc4c4c5ccccc5ccc43)cc2)c2ccc3ccccc3c2)cc1]

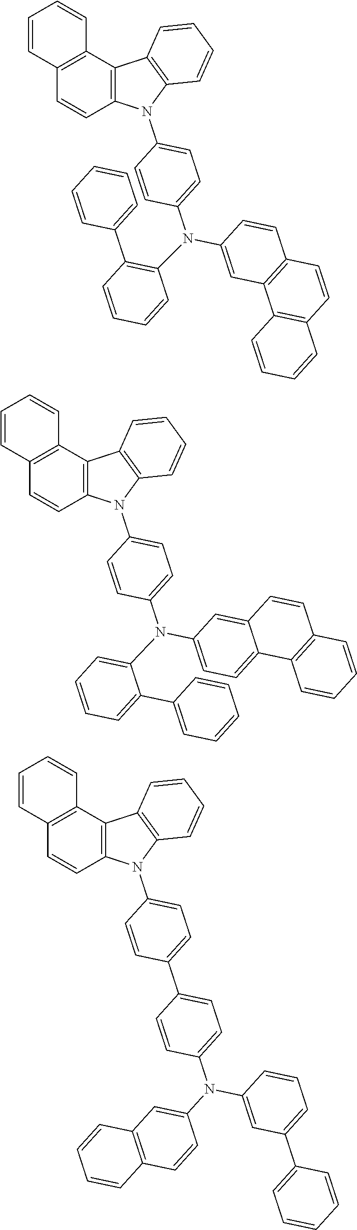

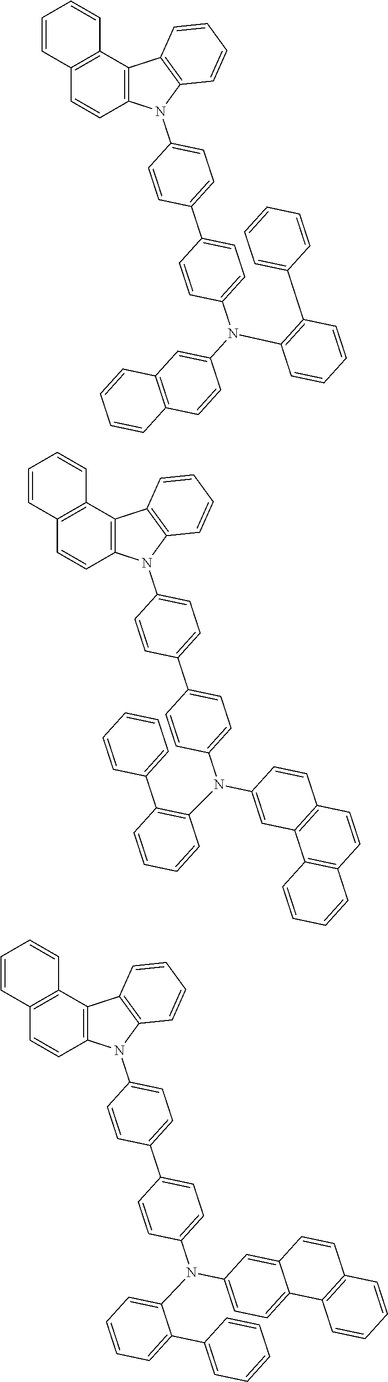 [SMILES: c1ccc(-c2ccccc2N(c2ccc(-c3ccc(-n4c5ccccc5c5c6ccccc6ccc54)cc3)cc2)c2ccc3ccccc3c2)cc1 ; c1ccc(-c2ccccc2N(c2ccc(-c3ccc(-n4c5ccccc5c5c6ccccc6ccc54)cc3)cc2)c2ccc3ccc4ccccc4c3c2)cc1 ; c1ccc(-c2ccccc2N(c2ccc(-c3ccc(-n4c5ccccc5c5c6ccccc6ccc54)cc3)cc2)c2ccc3c(ccc4ccccc43)c2)cc1]

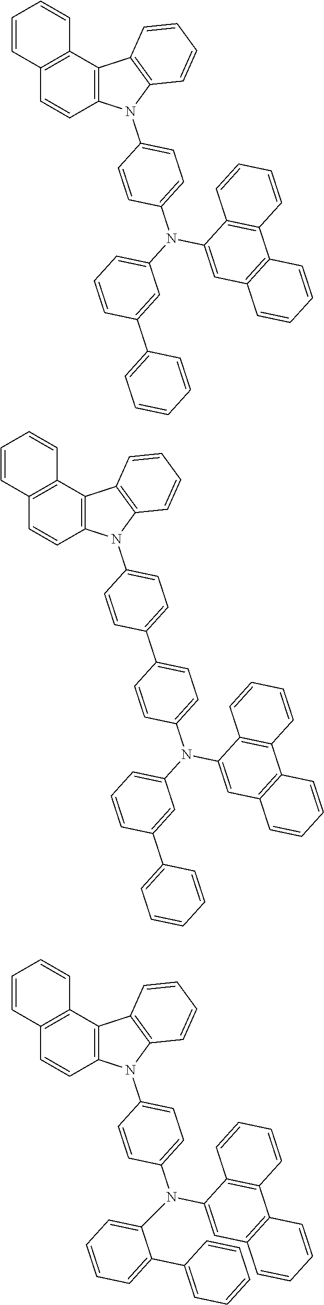

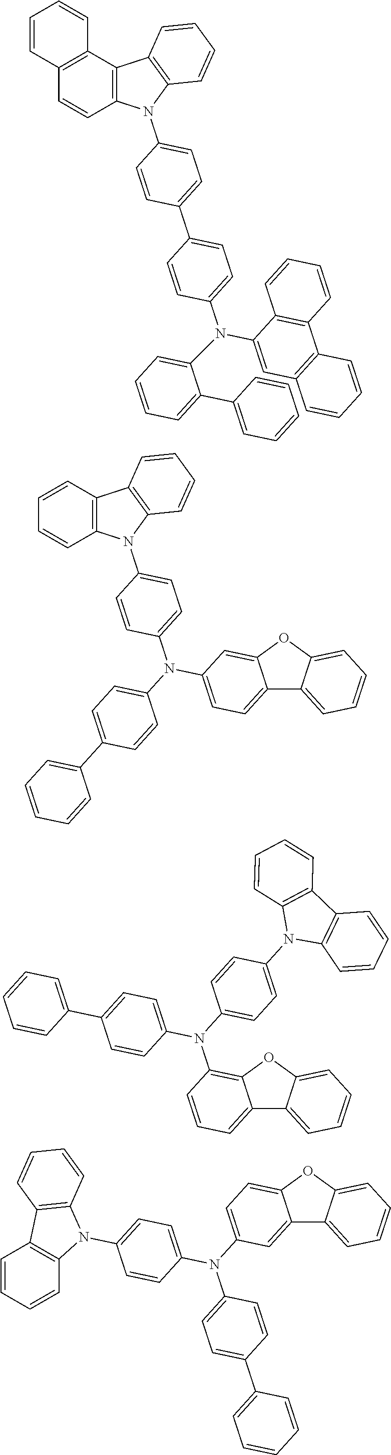

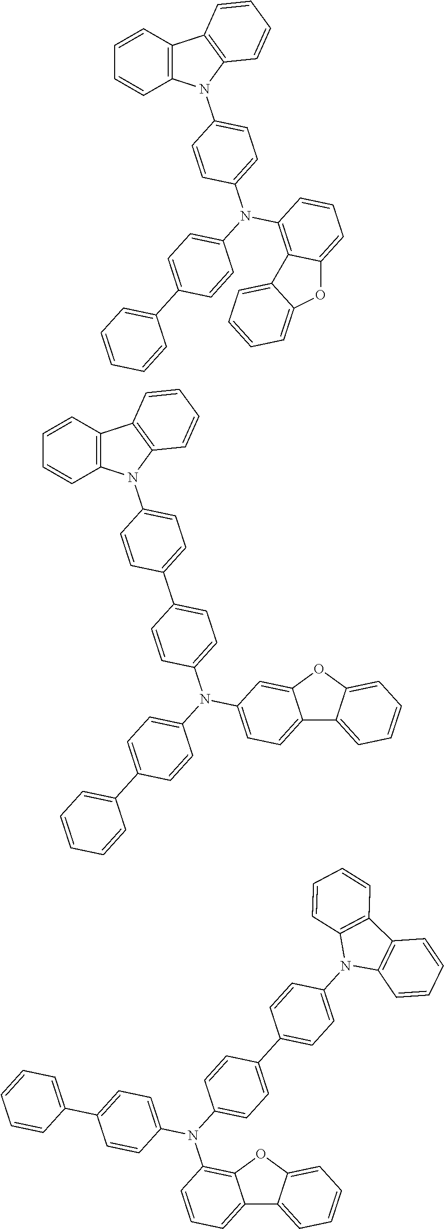

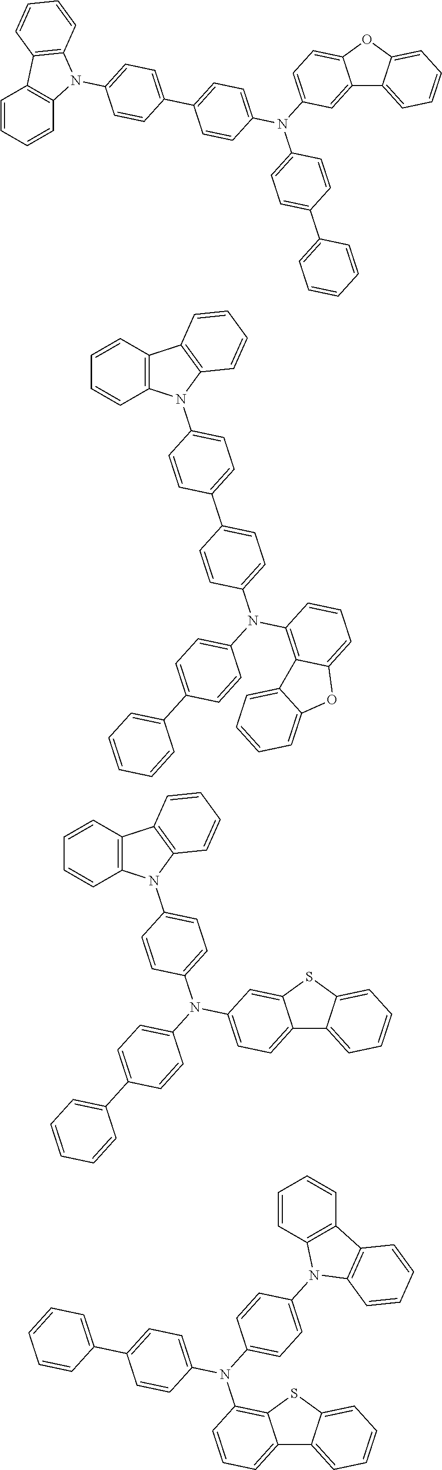

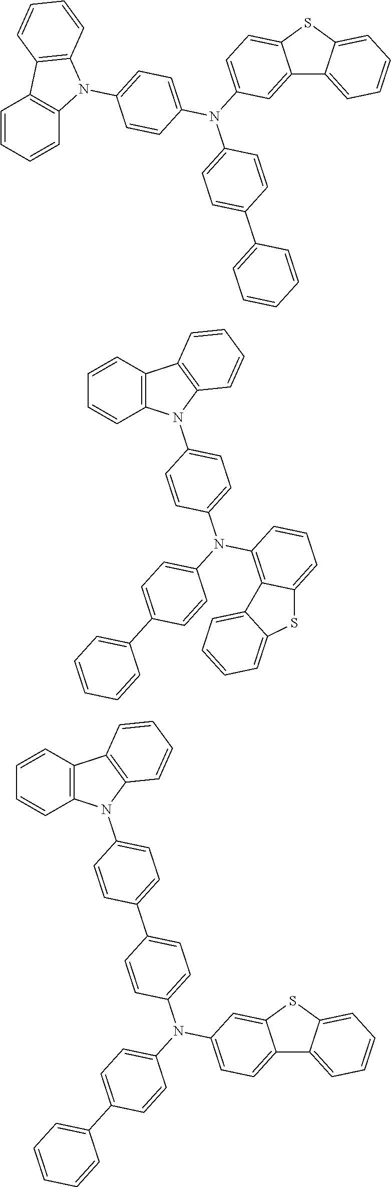

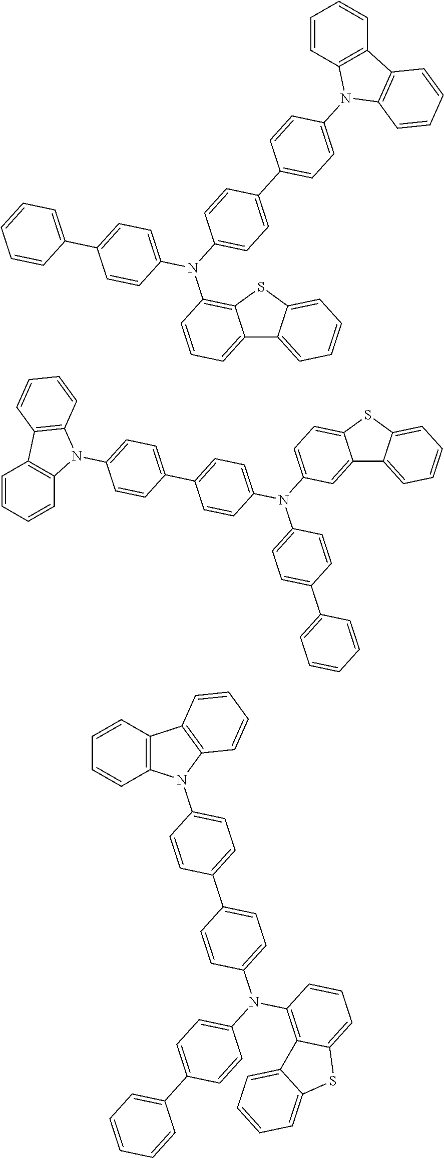 [SMILES: c1ccc(-c2ccc(N(c3ccc(-c4ccc(-n5c6ccccc6c6ccccc65)cc4)cc3)c3cccc4c3sc3ccccc34)cc2)cc1 ; c1ccc(-c2ccc(N(c3ccc(-c4ccc(-n5c6ccccc6c6ccccc65)cc4)cc3)c3ccc4sc5ccccc5c4c3)cc2)cc1 ; c1ccc(-c2ccc(N(c3ccc(-c4ccc(-n5c6ccccc6c6ccccc65)cc4)cc3)c3cccc4sc5ccccc5c34)cc2)cc1]

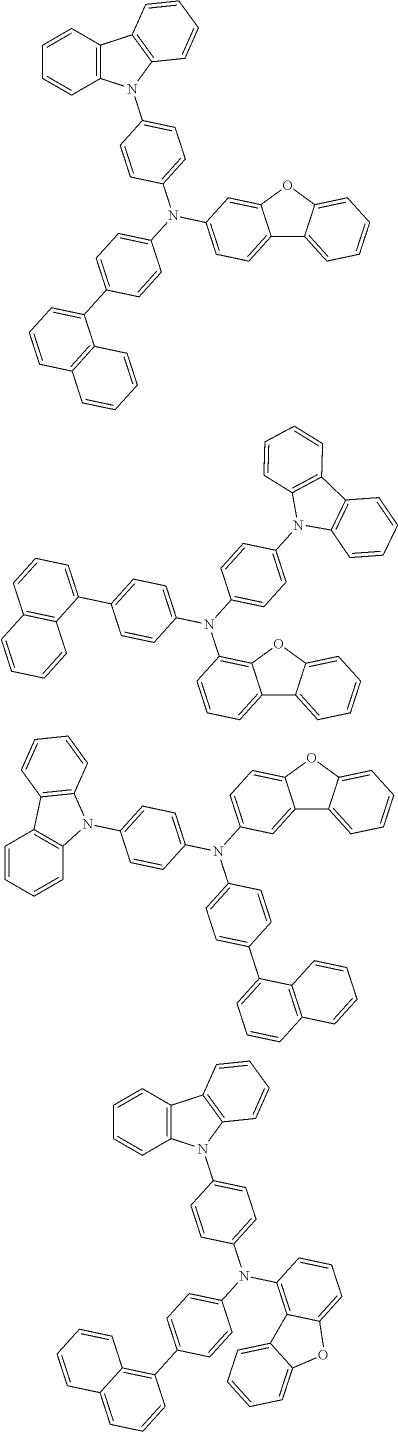

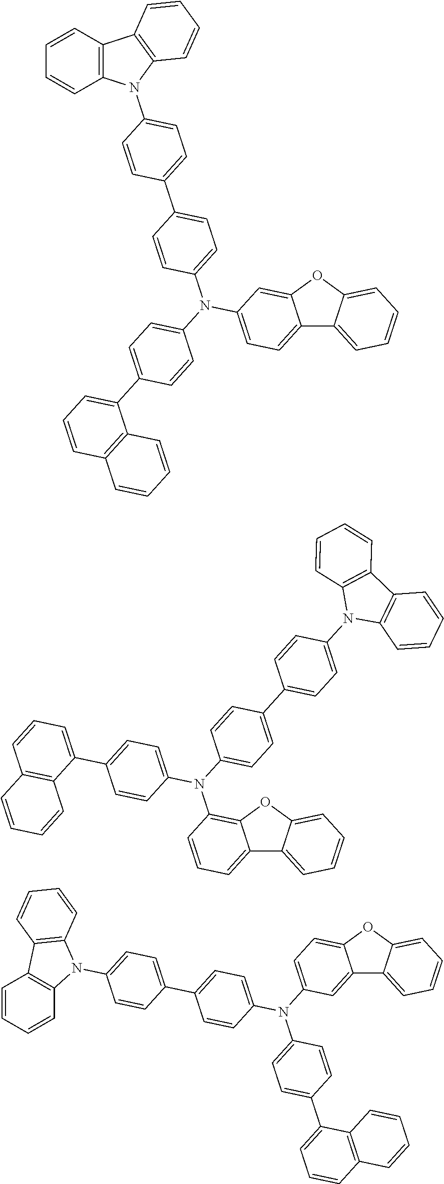 [SMILES: c1ccc2c(-c3ccc(N(c4ccc(-c5ccc(-n6c7ccccc7c7ccccc76)cc5)cc4)c4ccc5c(c4)oc4ccccc45)cc3)cccc2c1 ; c1ccc2c(-c3ccc(N(c4ccc(-c5ccc(-n6c7ccccc7c7ccccc76)cc5)cc4)c4cccc5c4oc4ccccc45)cc3)cccc2c1 ; c1ccc2c(-c3ccc(N(c4ccc(-c5ccc(-n6c7ccccc7c7ccccc76)cc5)cc4)c4ccc5oc6ccccc6c5c4)cc3)cccc2c1]

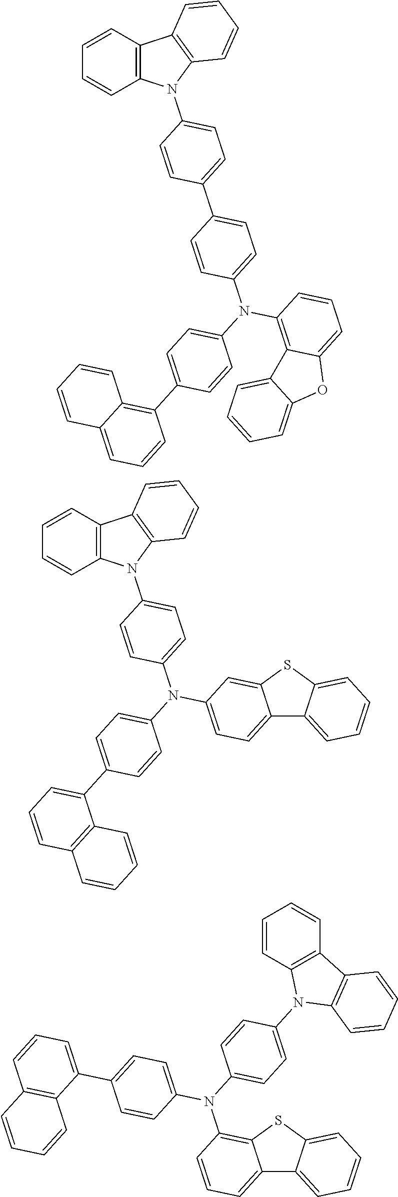

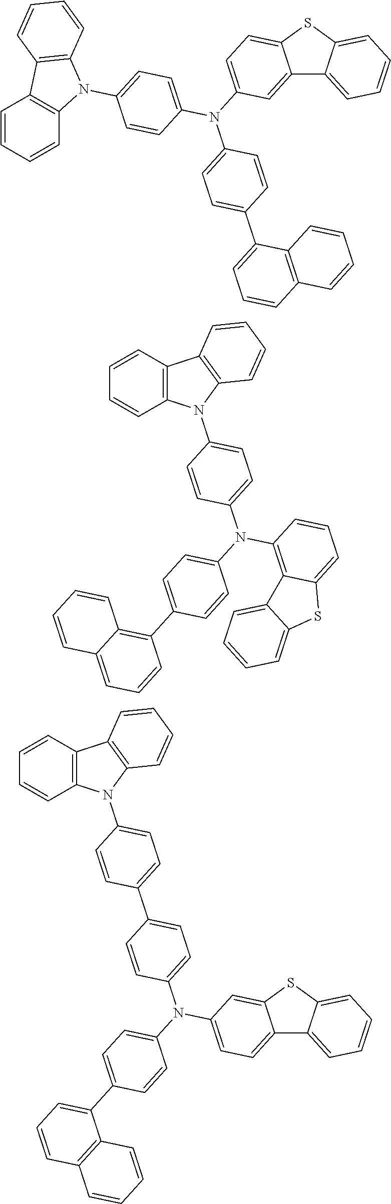

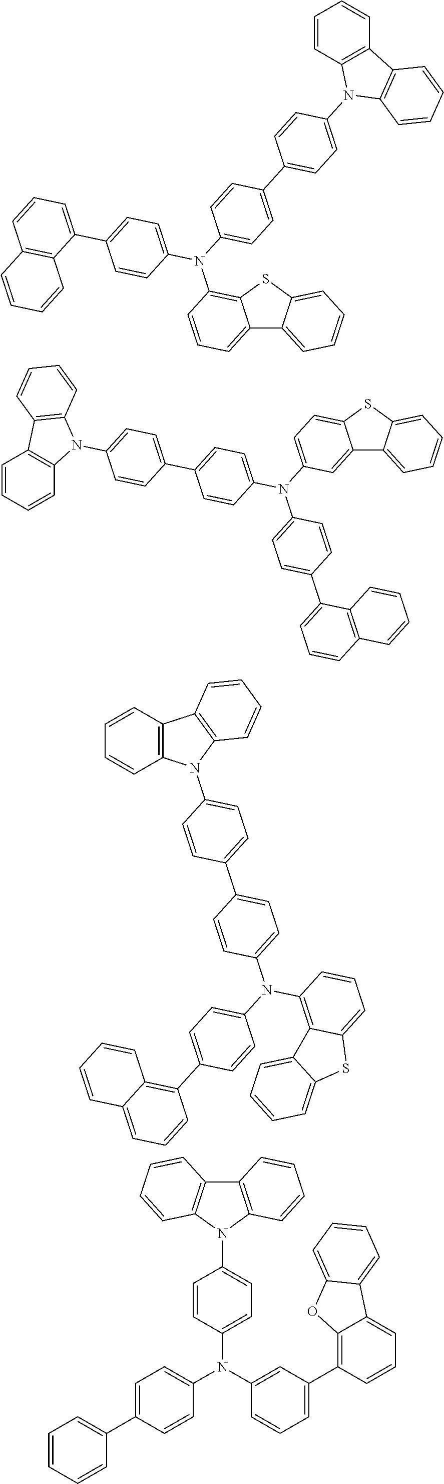

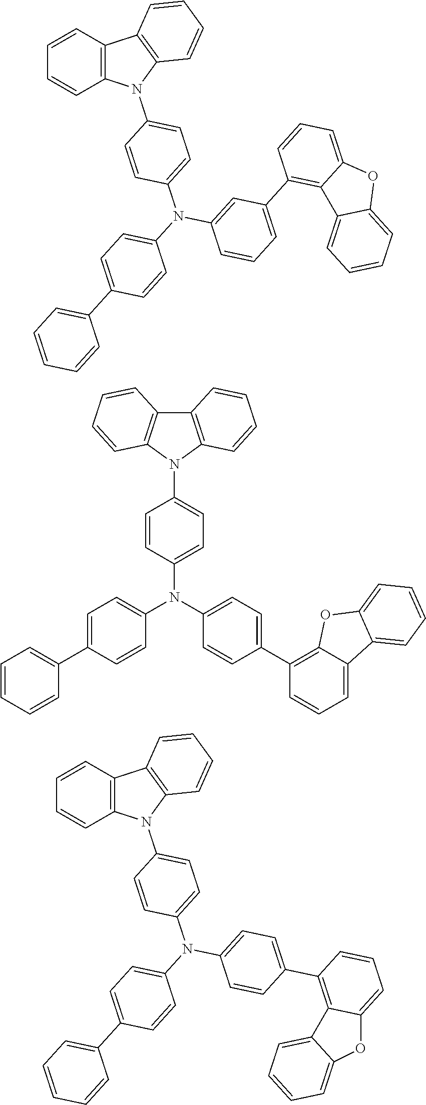

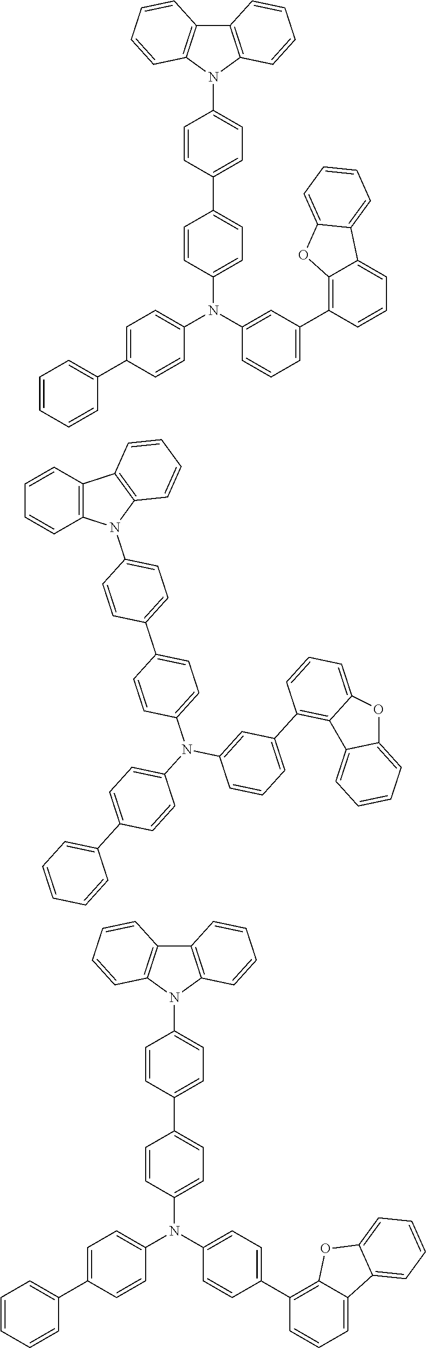

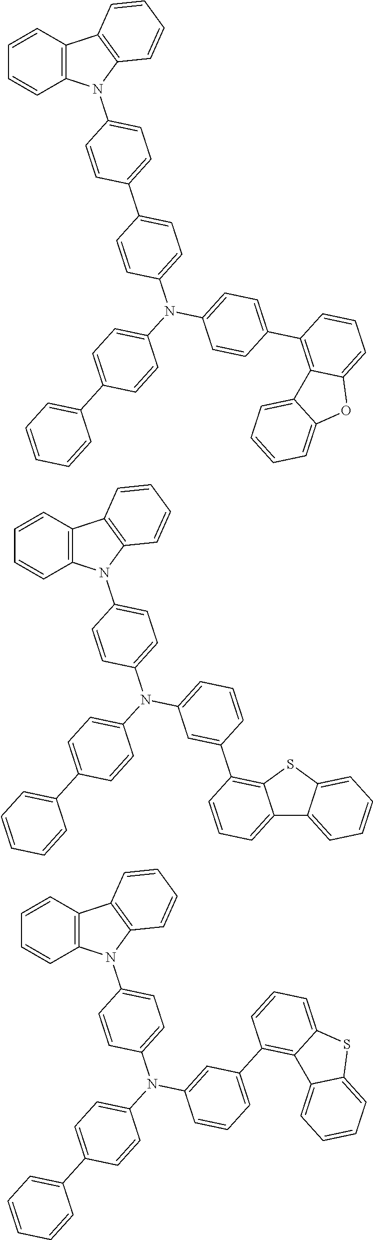

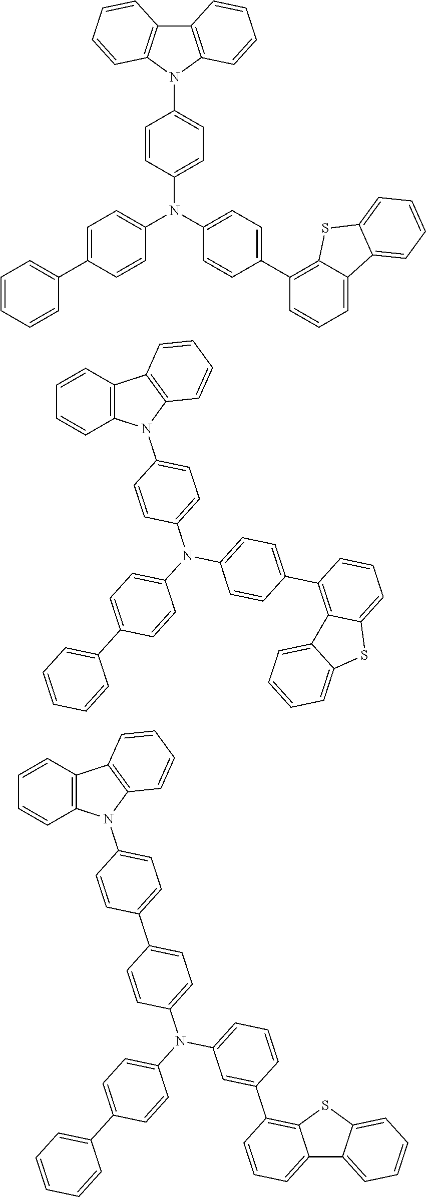

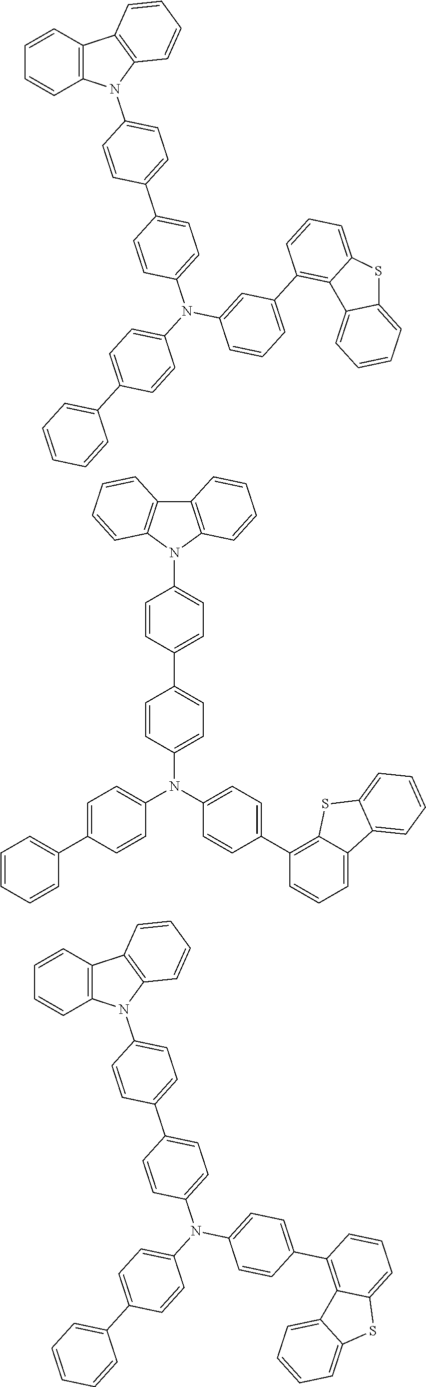

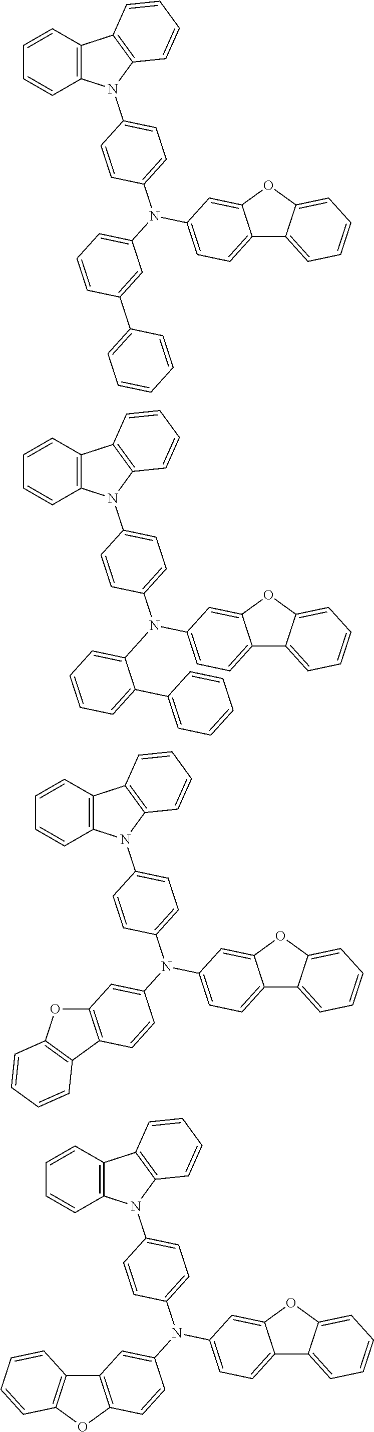

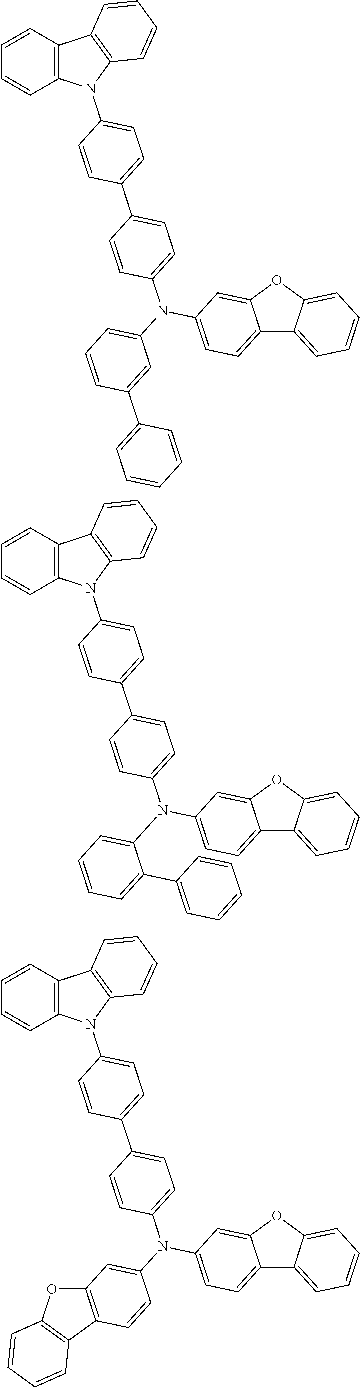

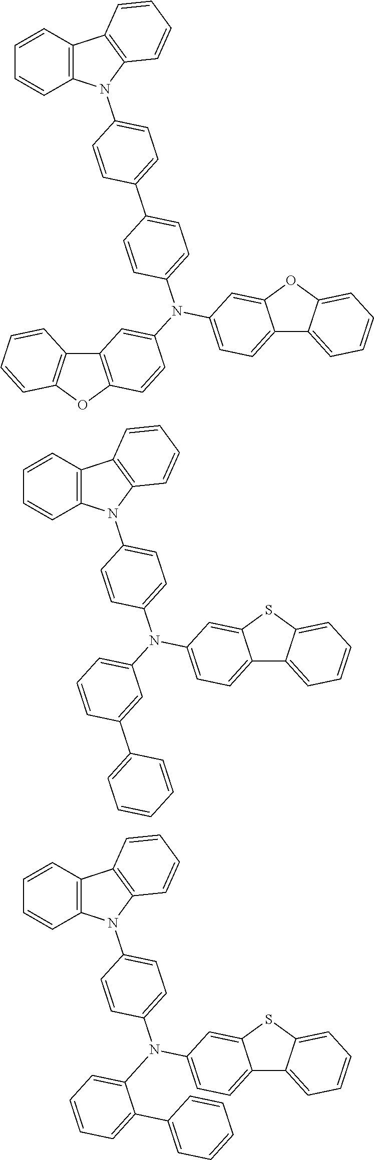

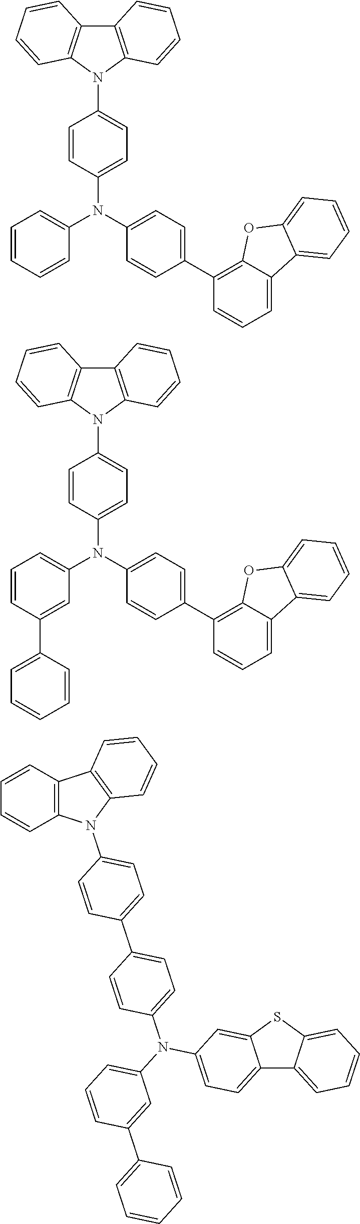

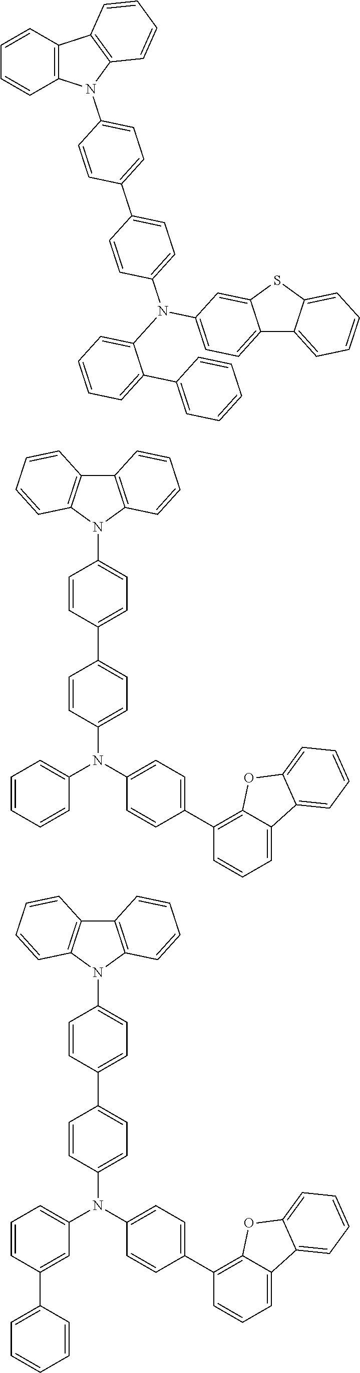 [SMILES: c1ccc(-c2ccccc2N(c2ccc(-c3ccc(-n4c5ccccc5c5ccccc54)cc3)cc2)c2ccc3c(c2)sc2ccccc23)cc1 ; c1ccc(N(c2ccc(-c3ccc(-n4c5ccccc5c5ccccc54)cc3)cc2)c2ccc(-c3cccc4c3oc3ccccc34)cc2)cc1 ; c1ccc(-c2cccc(N(c3ccc(-c4ccc(-n5c6ccccc6c6ccccc65)cc4)cc3)c3ccc(-c4cccc5c4oc4ccccc45)cc3)c2)cc1]

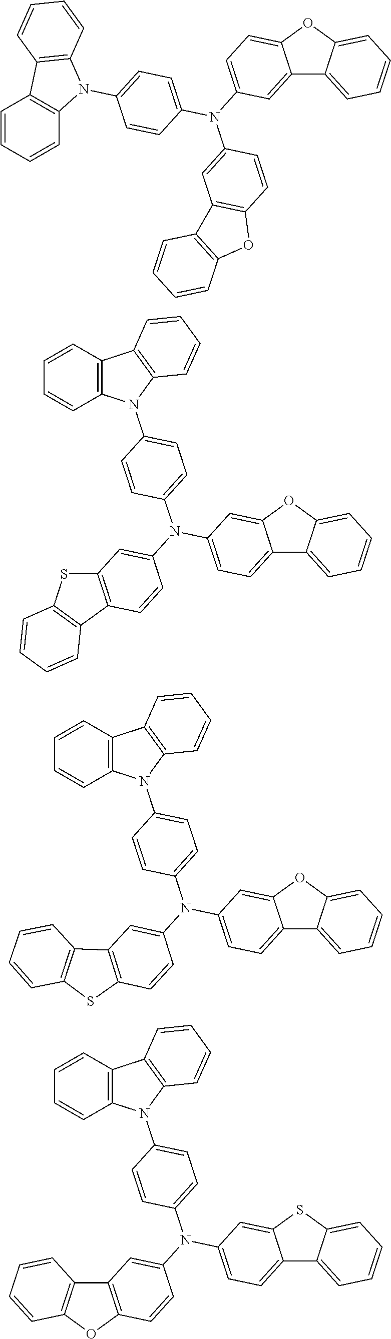

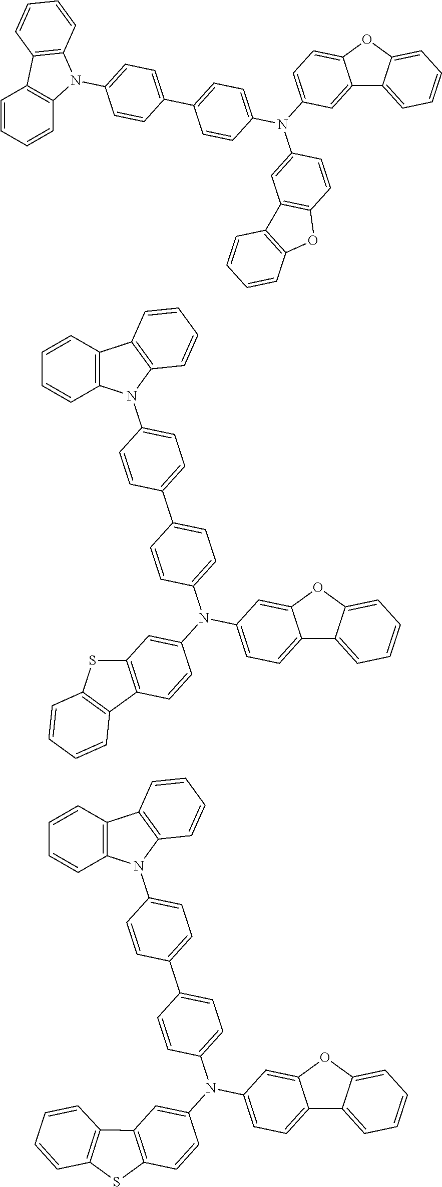 [SMILES: c1ccc2c(c1)oc1ccc(N(c3ccc(-c4ccc(-n5c6ccccc6c6ccccc65)cc4)cc3)c3ccc4oc5ccccc5c4c3)cc12 ; c1ccc2c(c1)oc1cc(N(c3ccc(-c4ccc(-n5c6ccccc6c6ccccc65)cc4)cc3)c3ccc4c(c3)sc3ccccc34)ccc12 ; c1ccc2c(c1)oc1cc(N(c3ccc(-c4ccc(-n5c6ccccc6c6ccccc65)cc4)cc3)c3ccc4sc5ccccc5c4c3)ccc12]

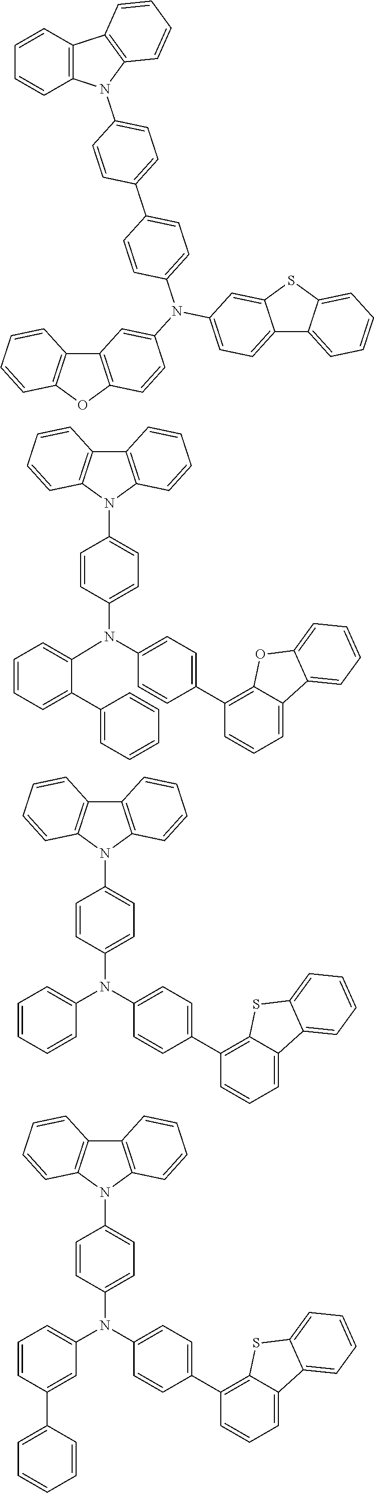

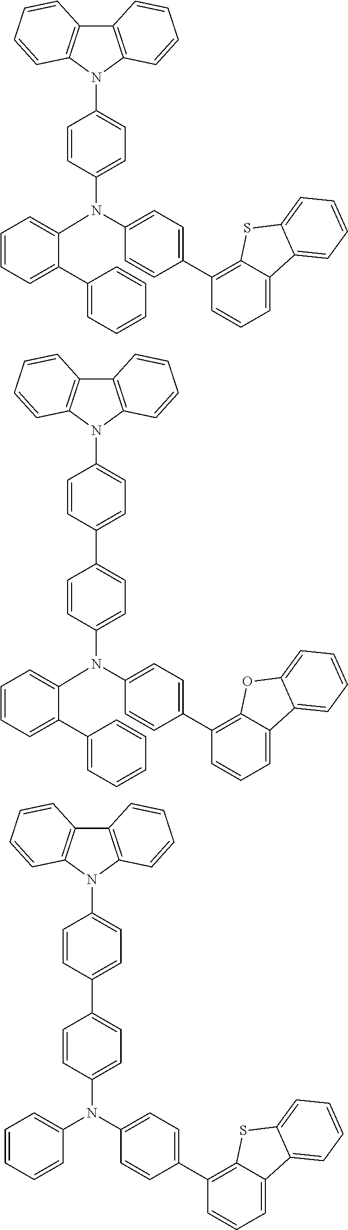

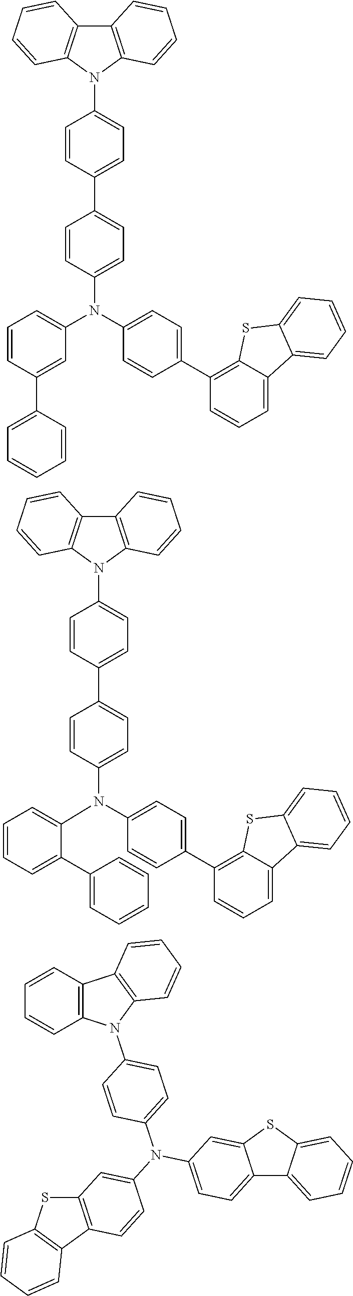

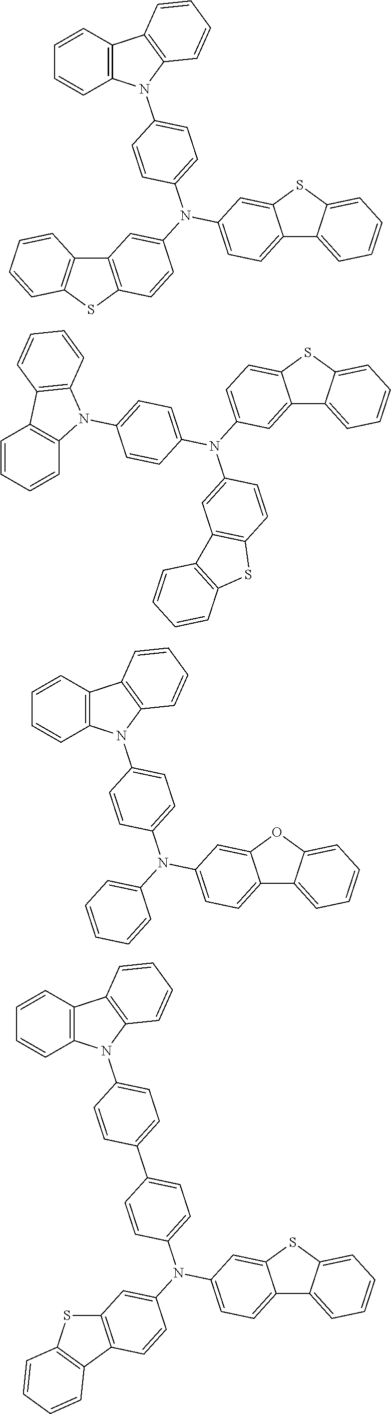

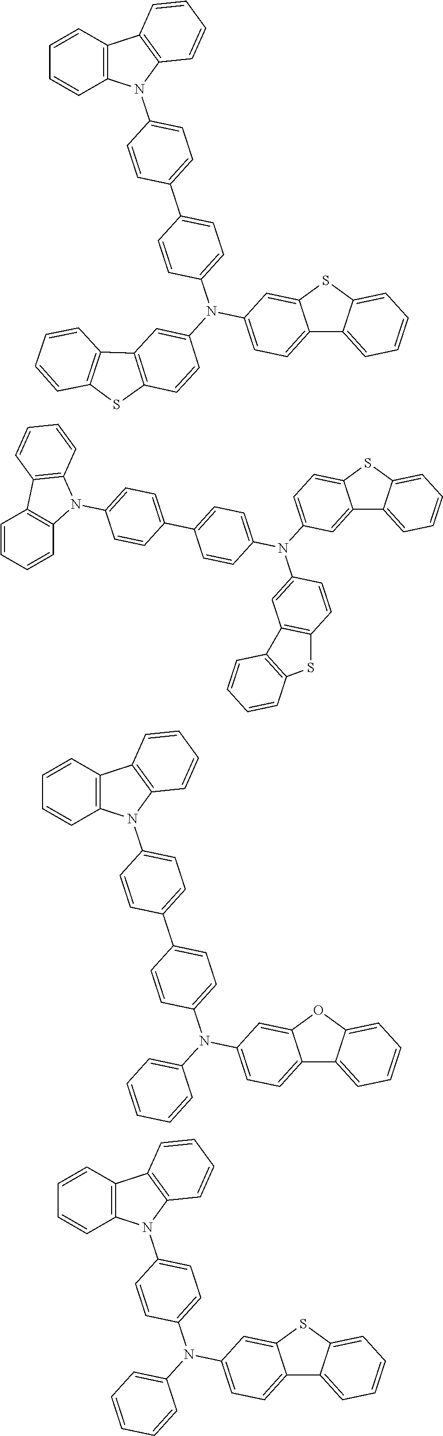

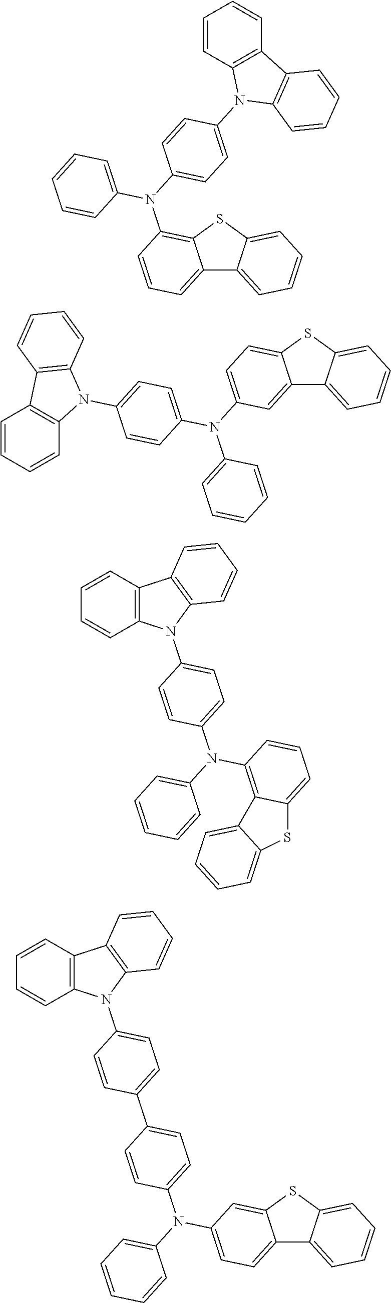

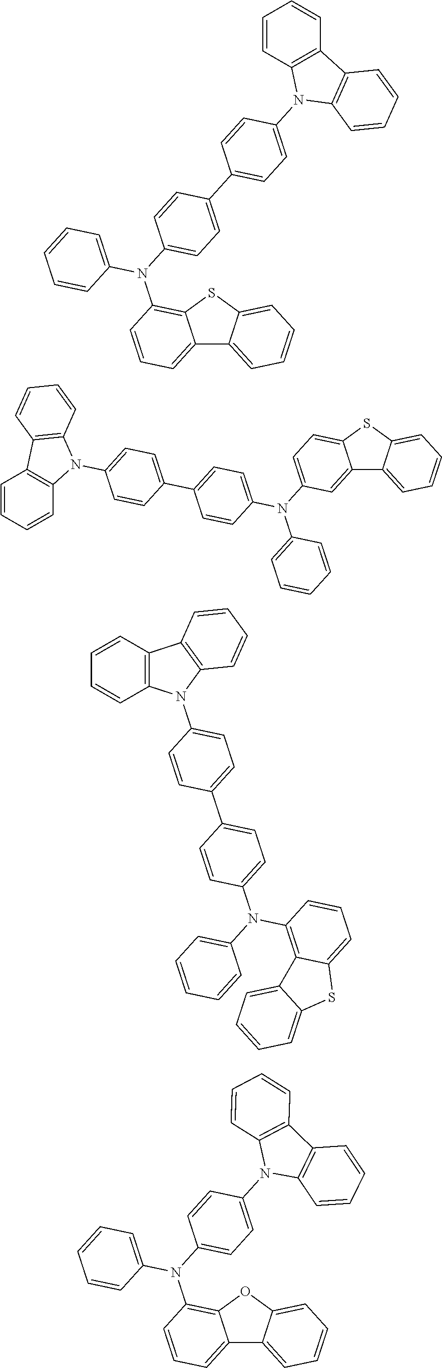

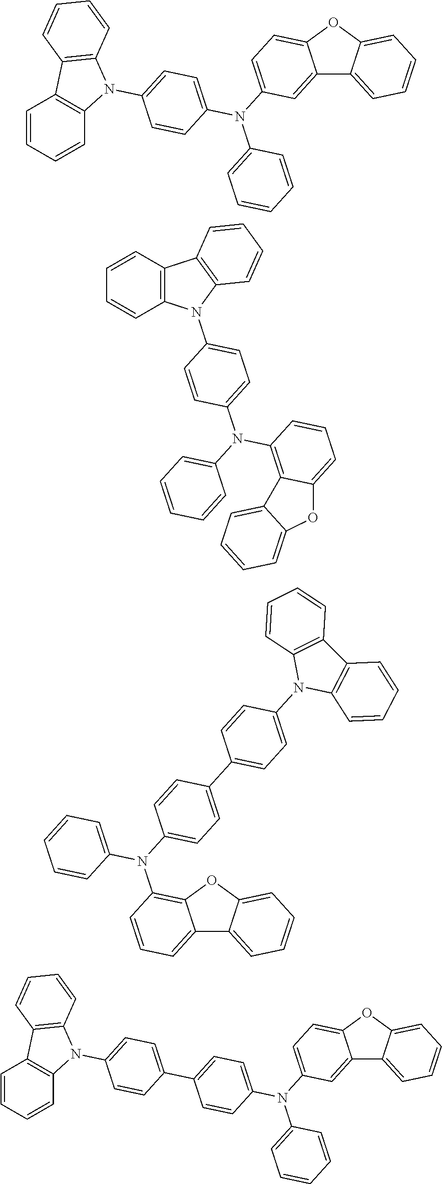

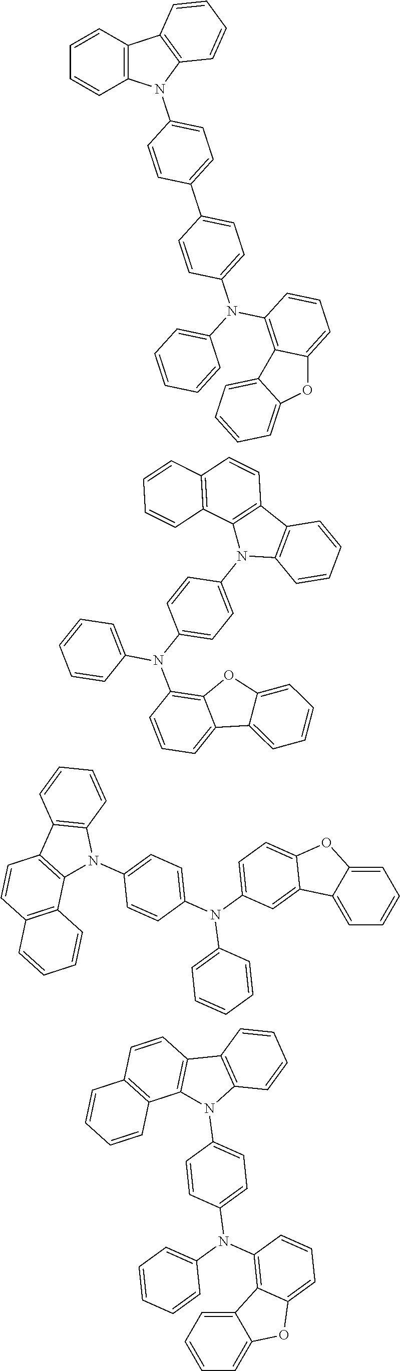

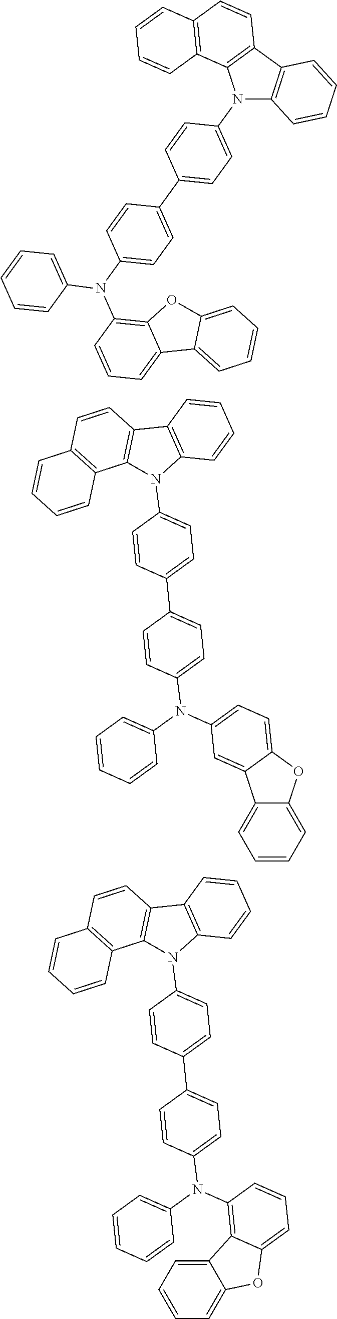

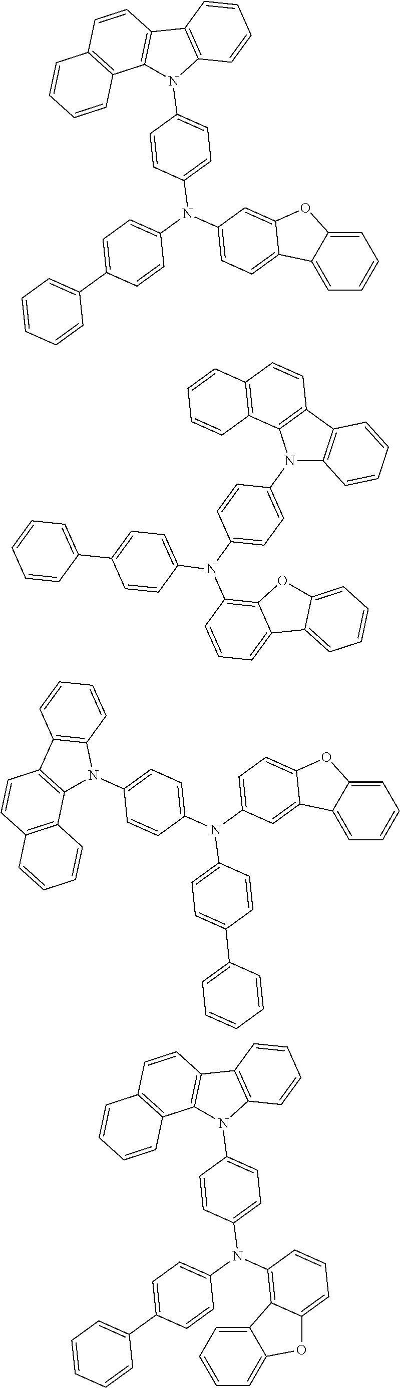

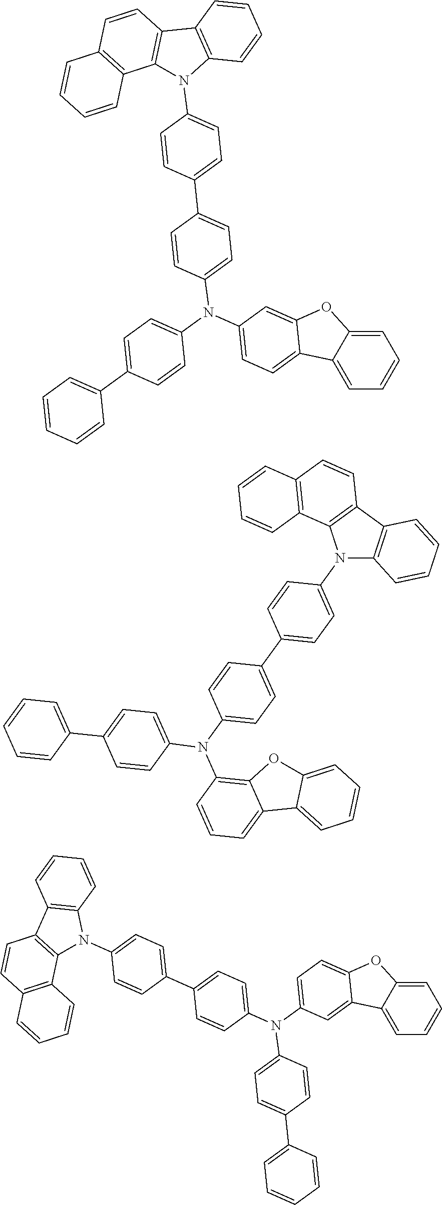 [SMILES: c1ccc(-c2ccc(N(c3ccc(-c4ccc(-n5c6ccccc6c6ccc7ccccc7c65)cc4)cc3)c3ccc4c(c3)oc3ccccc34)cc2)cc1 ; c1ccc(-c2ccc(N(c3ccc(-c4ccc(-n5c6ccccc6c6ccc7ccccc7c65)cc4)cc3)c3cccc4c3oc3ccccc34)cc2)cc1 ; c1ccc(-c2ccc(N(c3ccc(-c4ccc(-n5c6ccccc6c6ccc7ccccc7c65)cc4)cc3)c3ccc4oc5ccccc5c4c3)cc2)cc1]

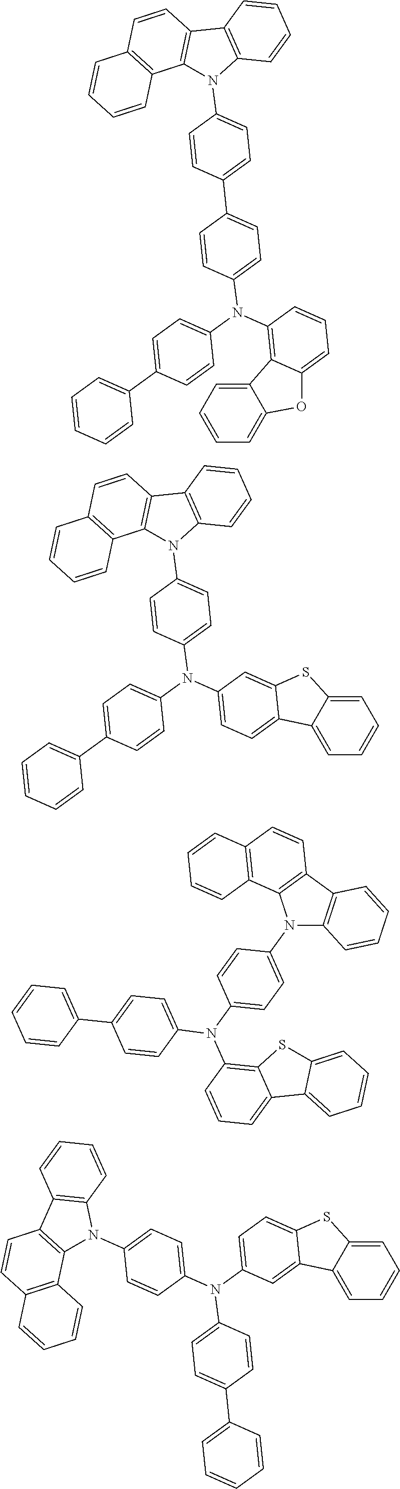

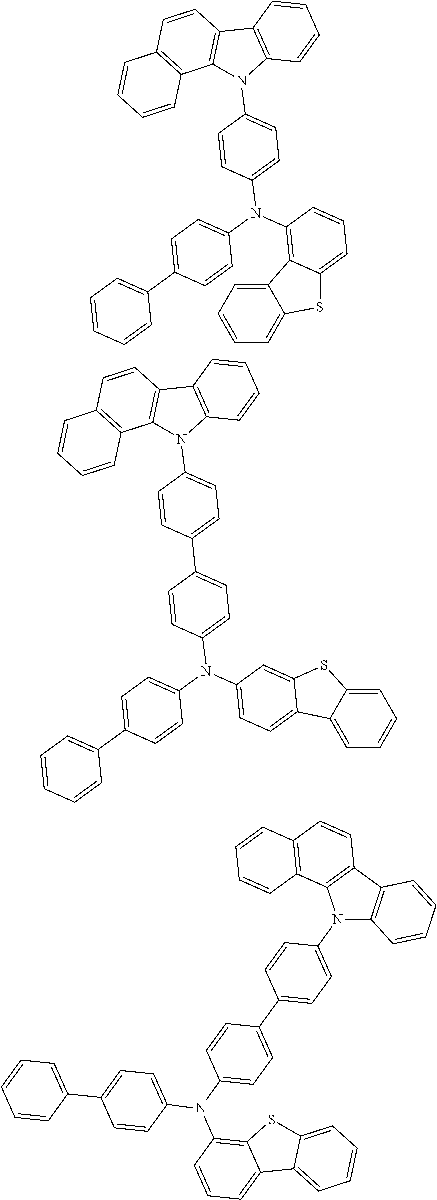

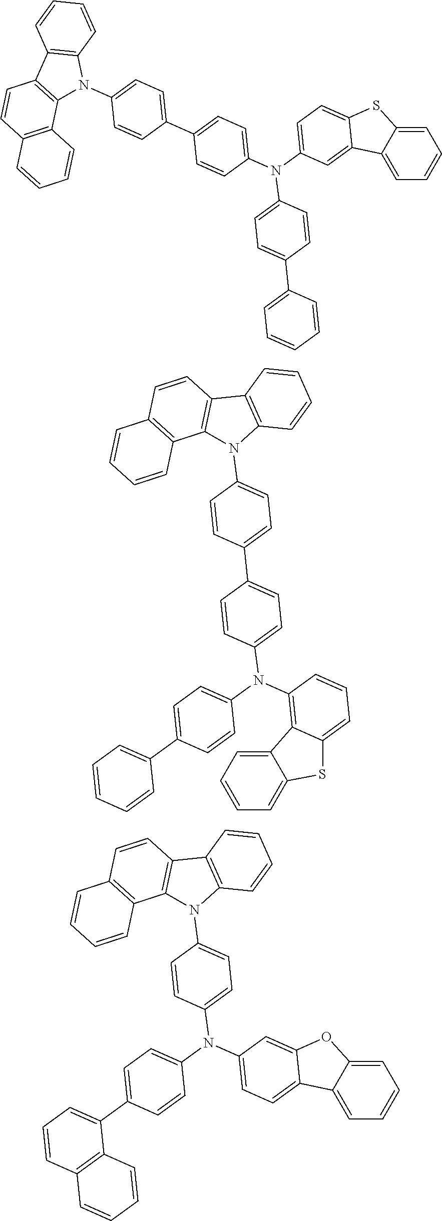

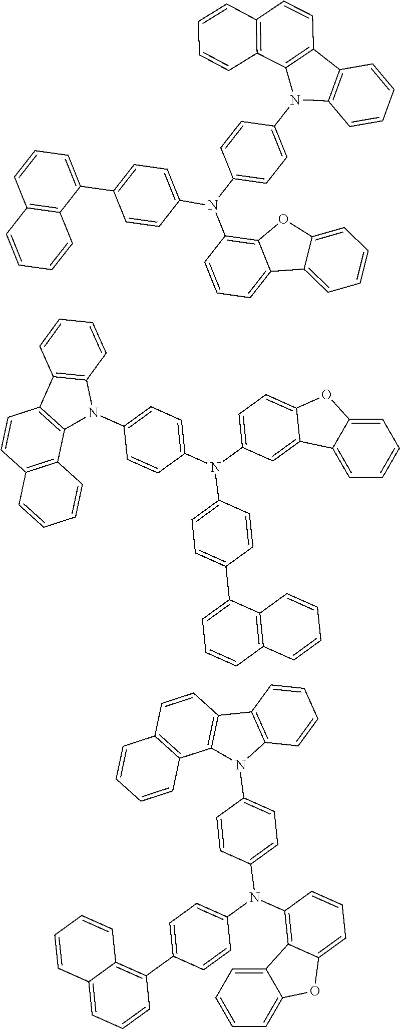

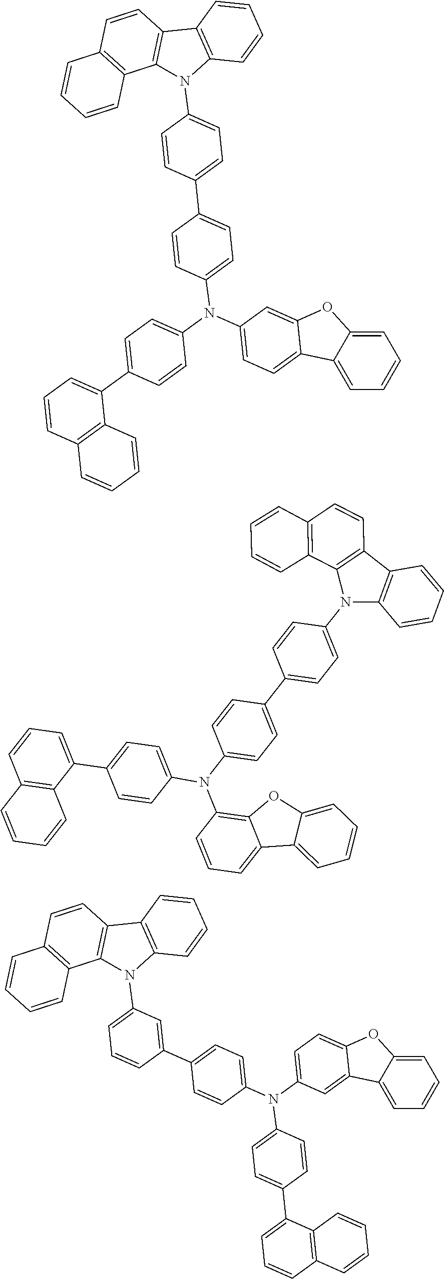

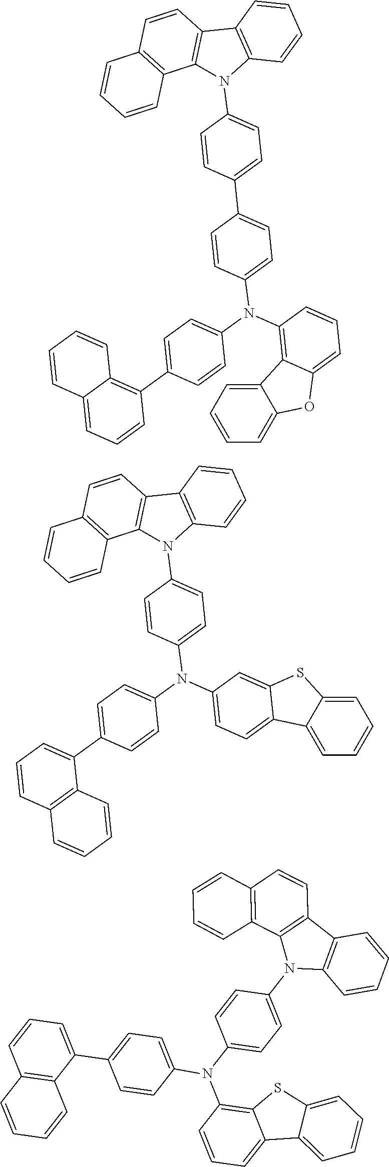

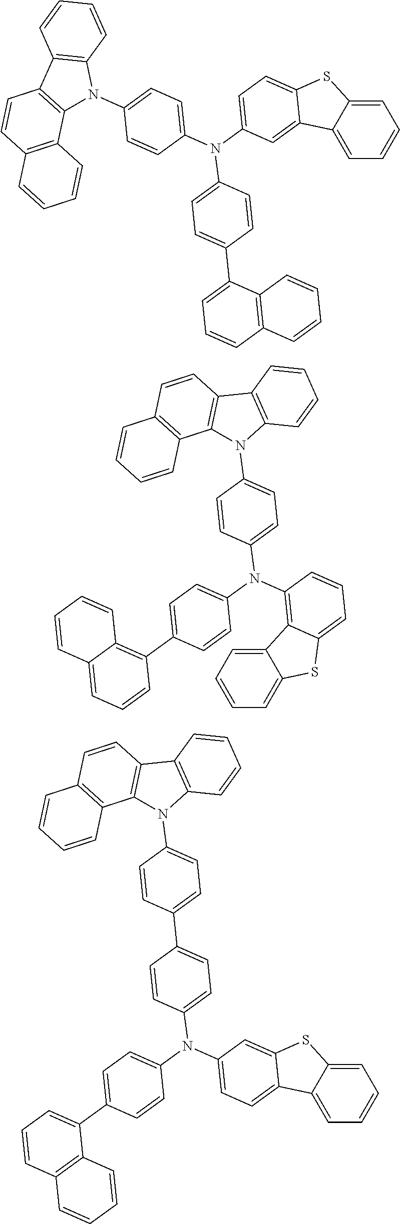

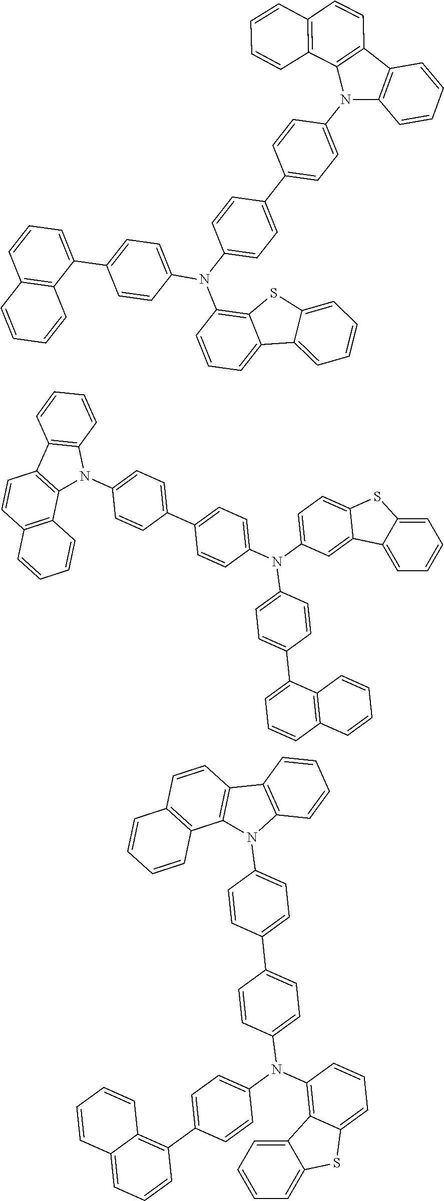

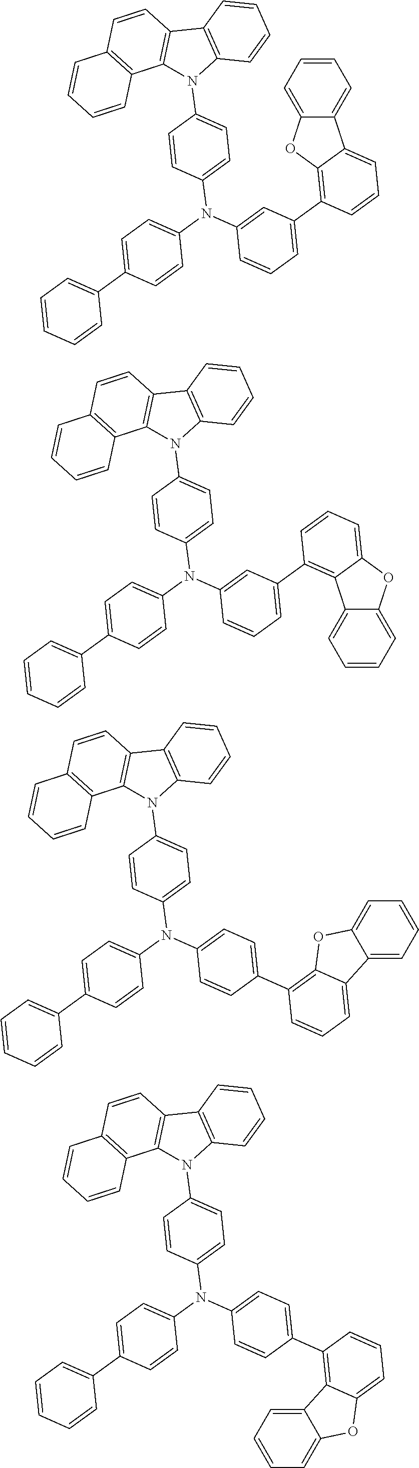

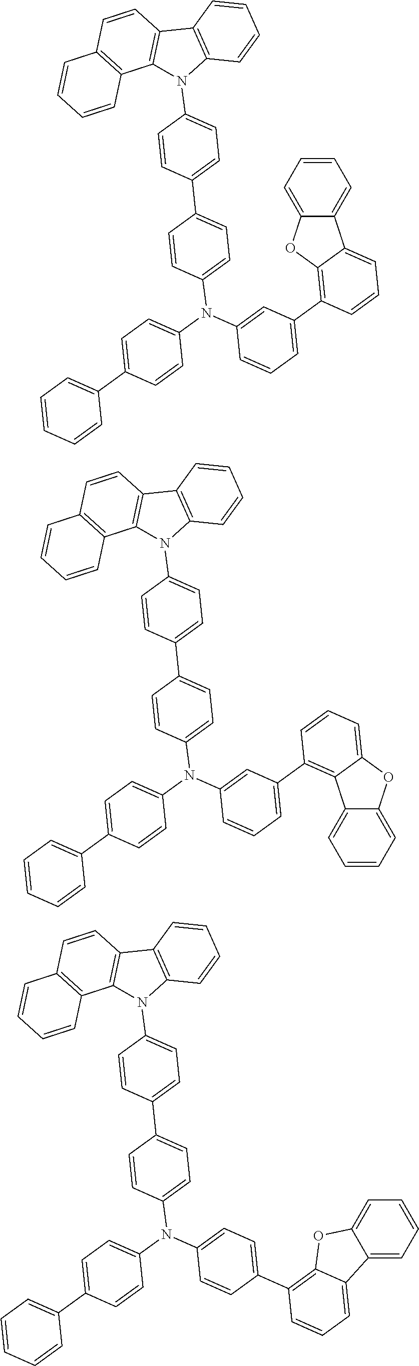

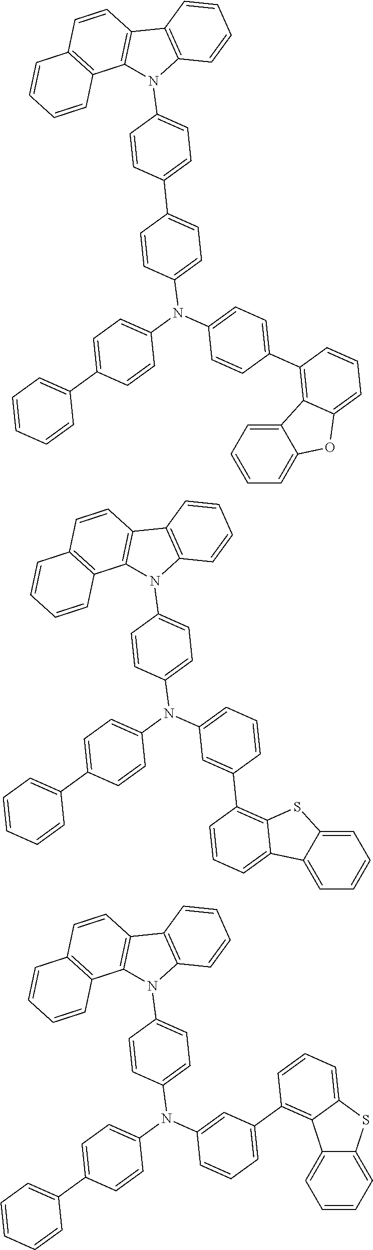

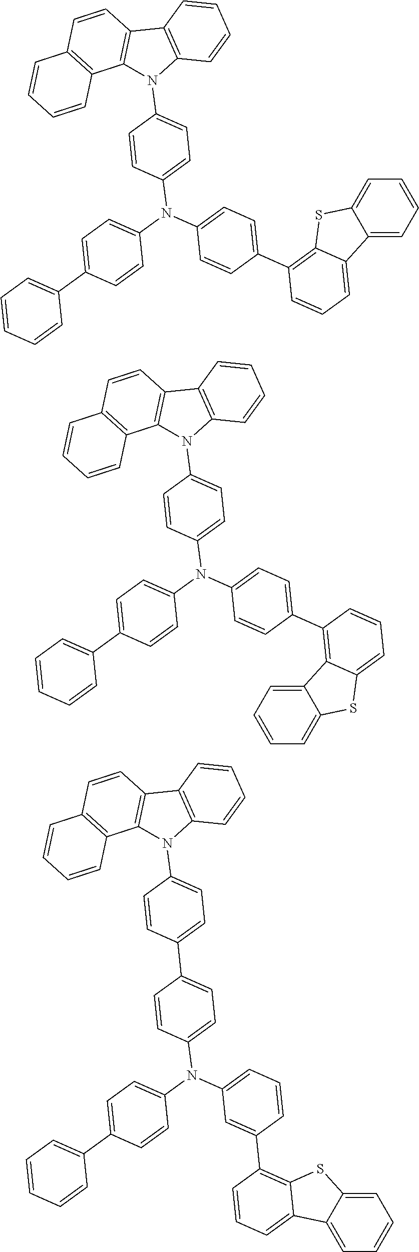

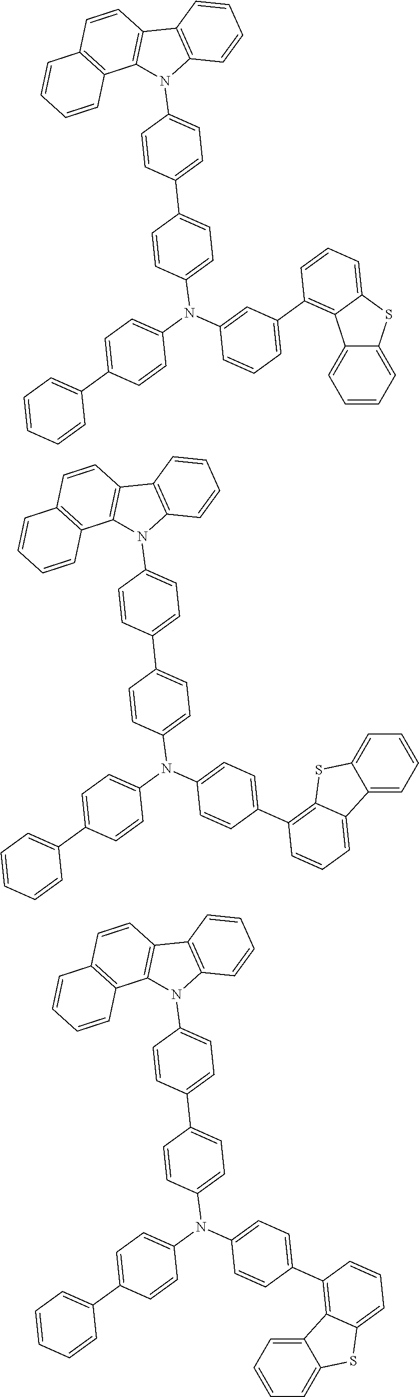

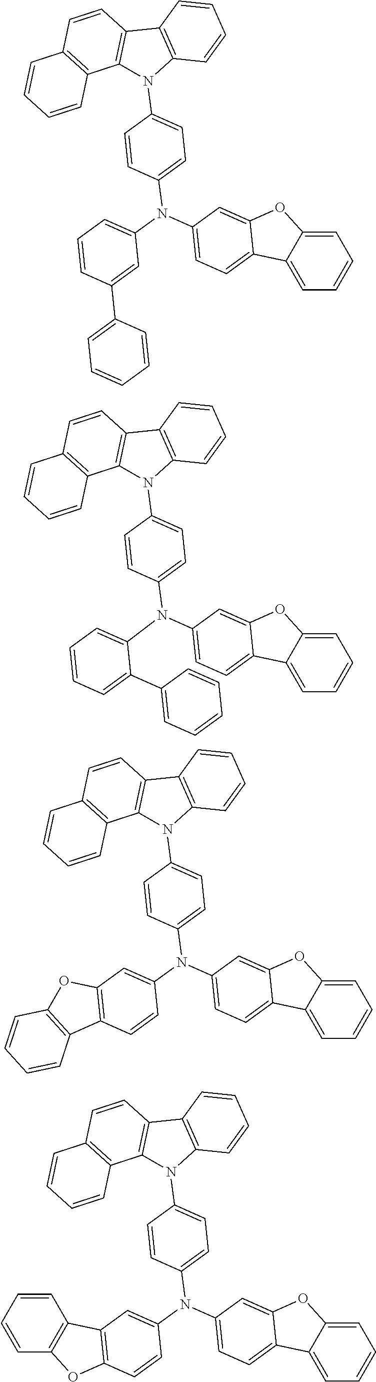

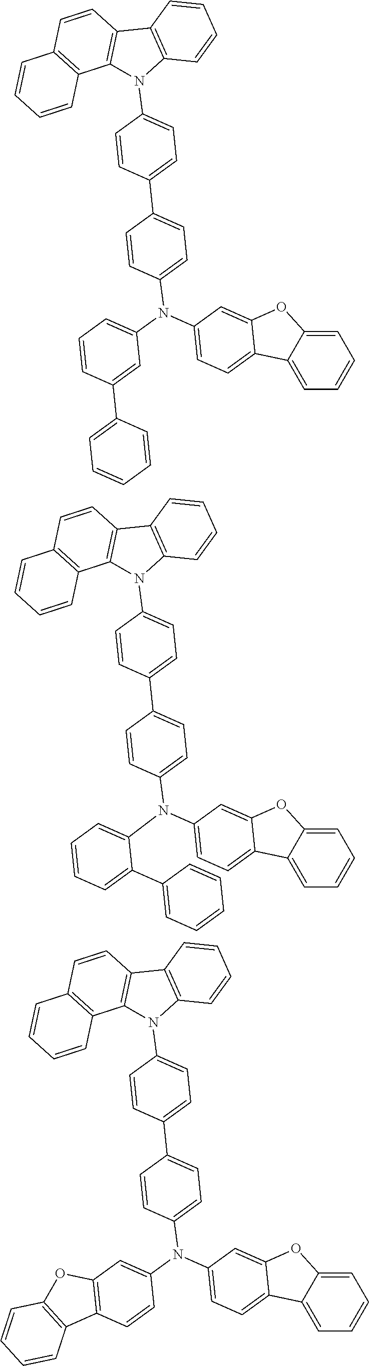

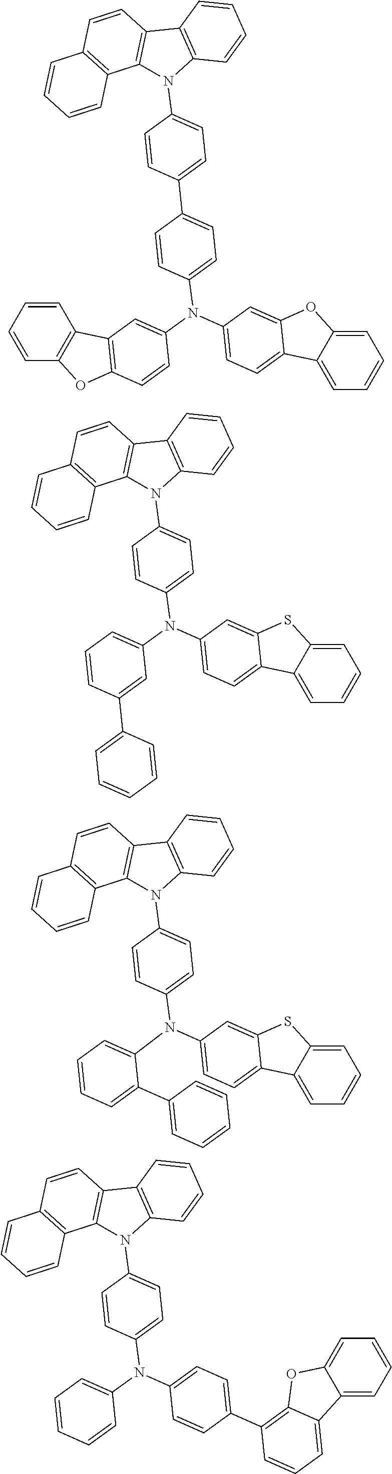

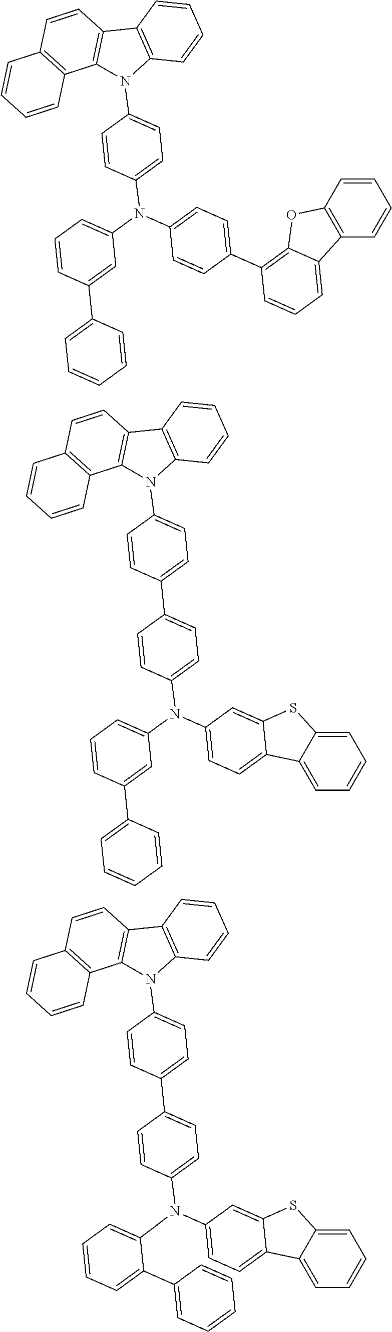

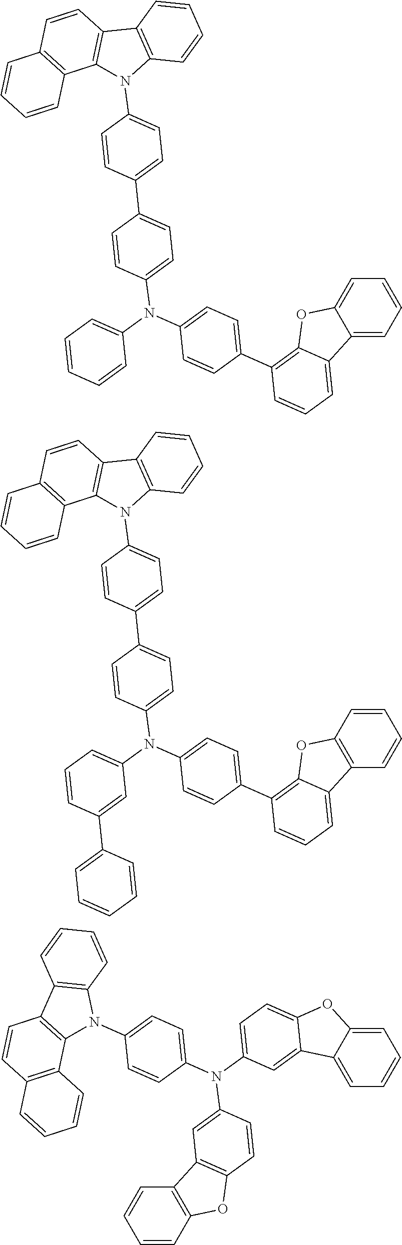

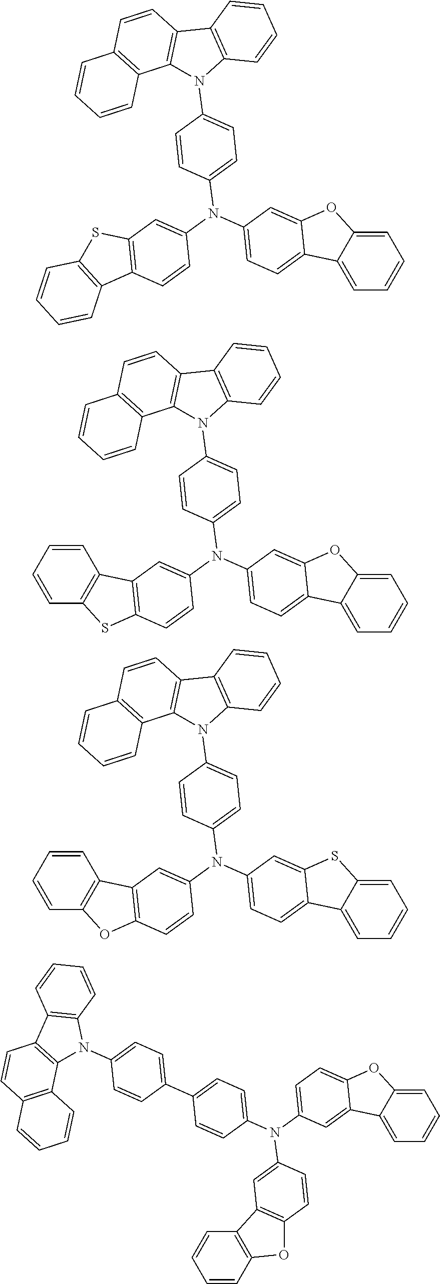

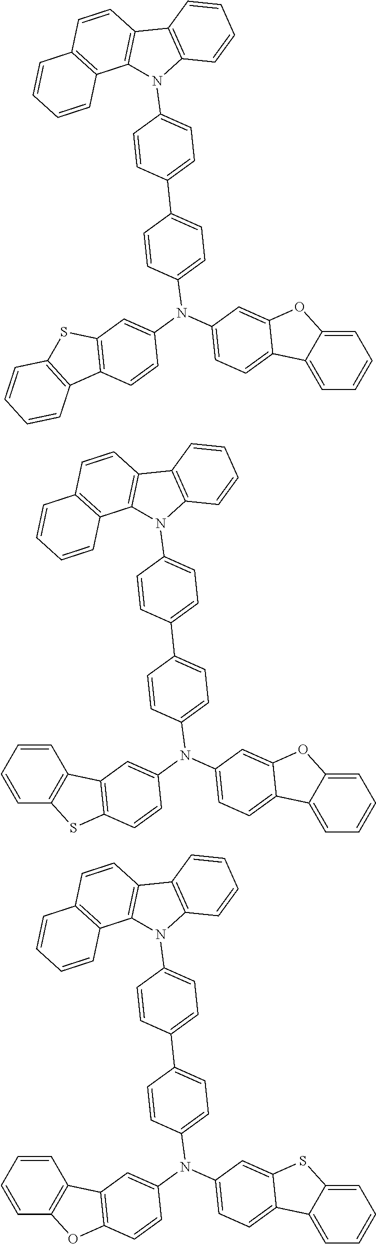

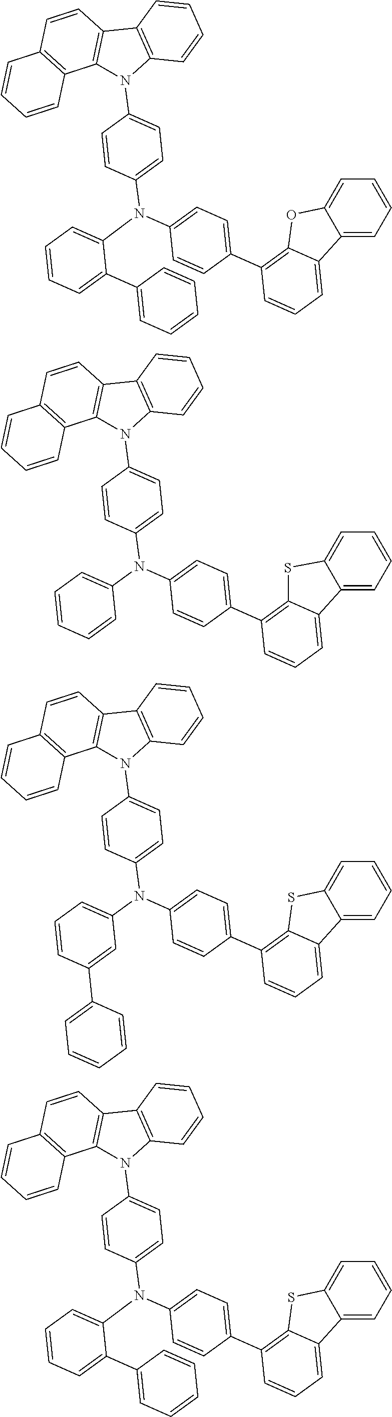

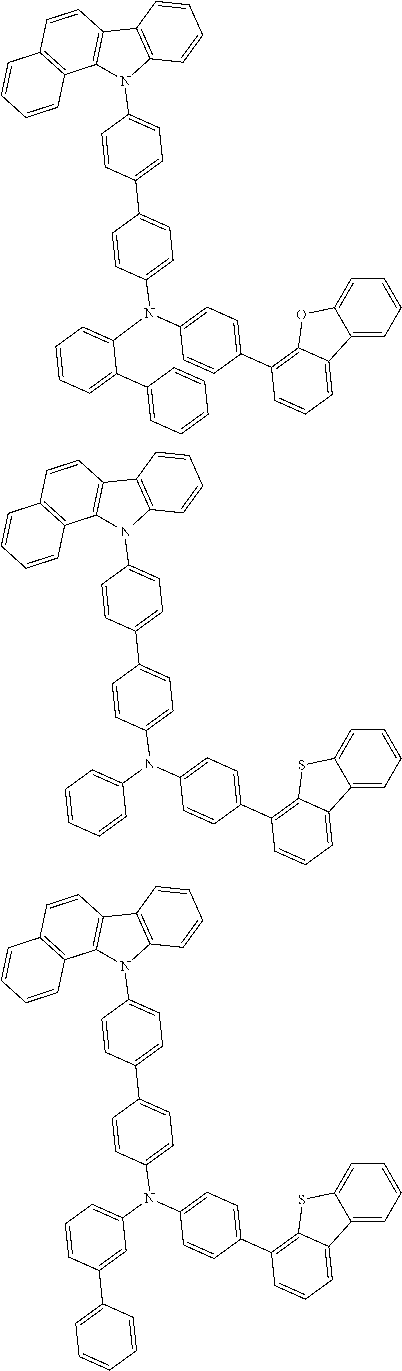

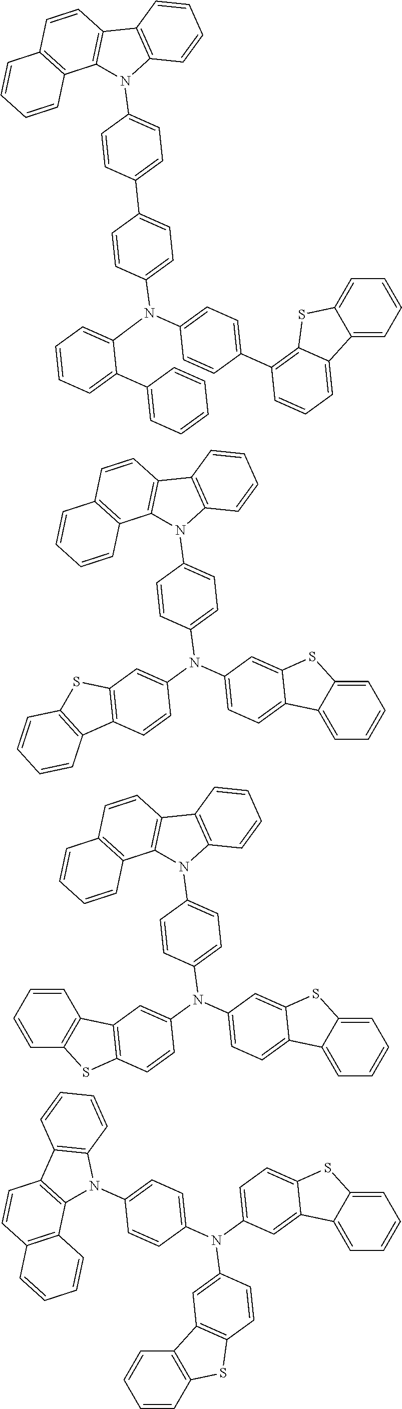

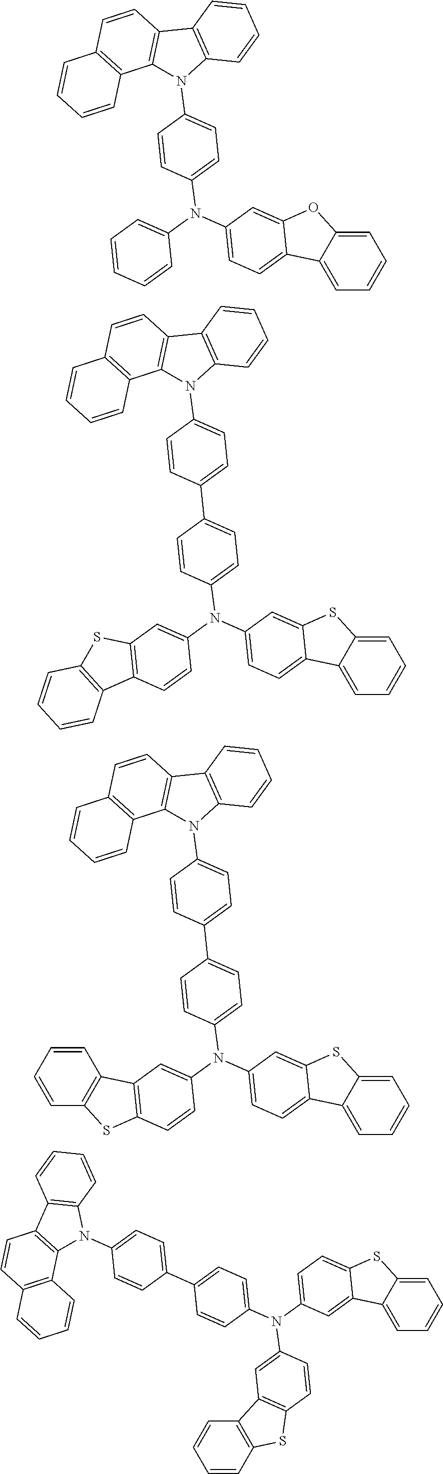

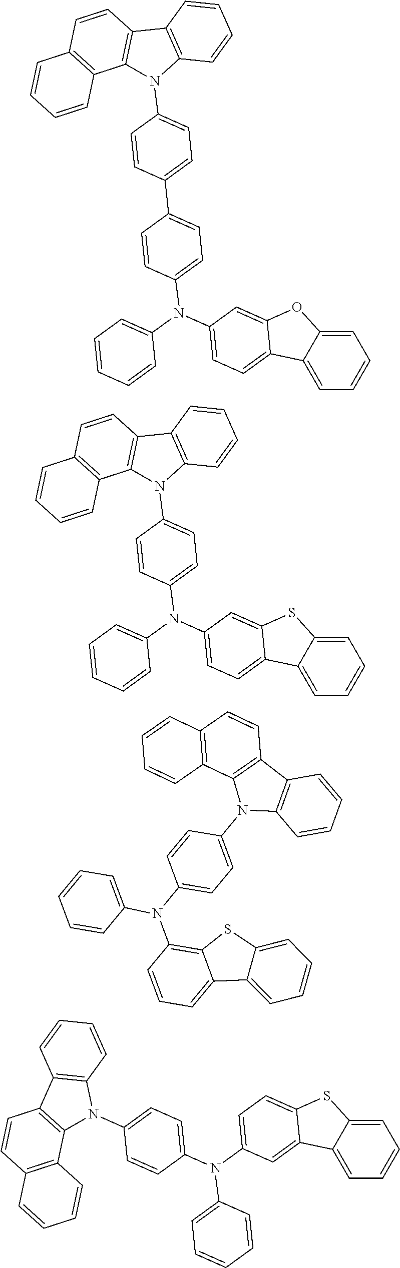

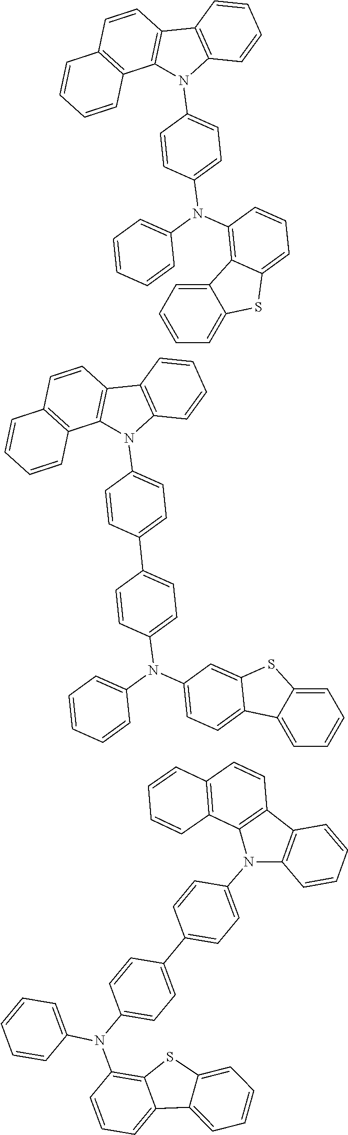

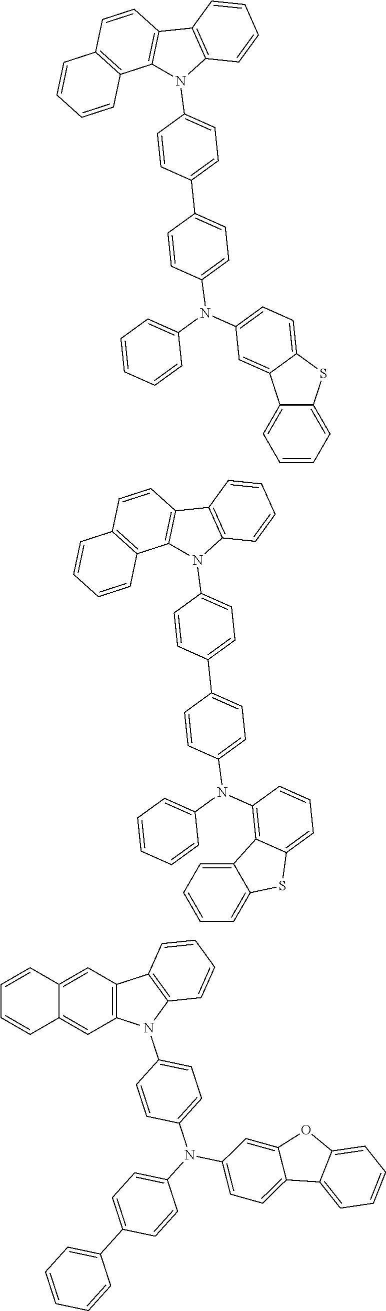

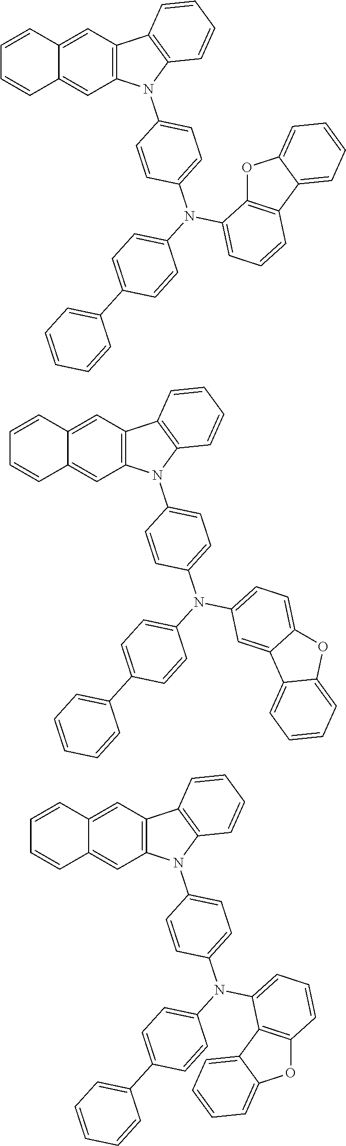

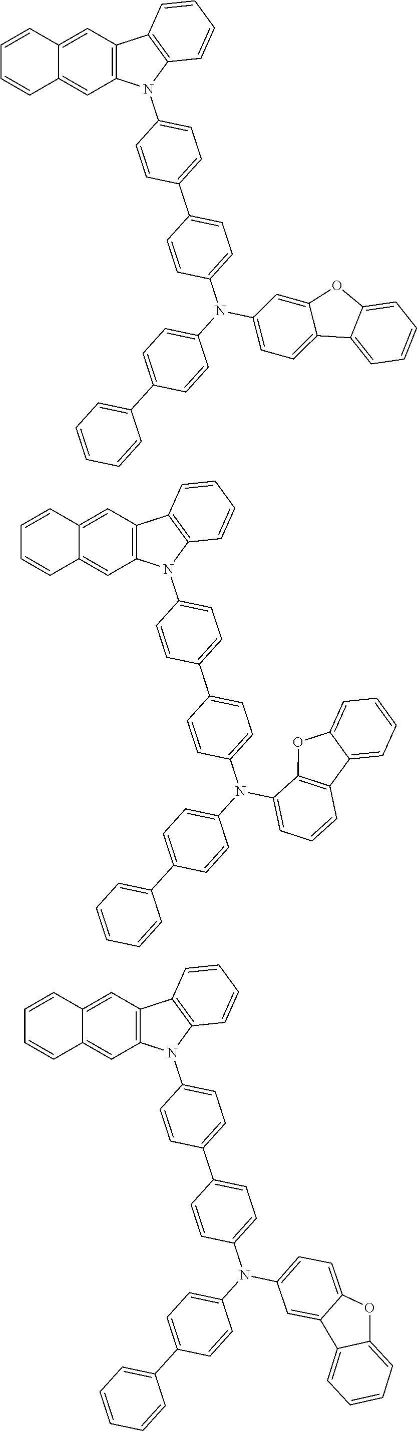

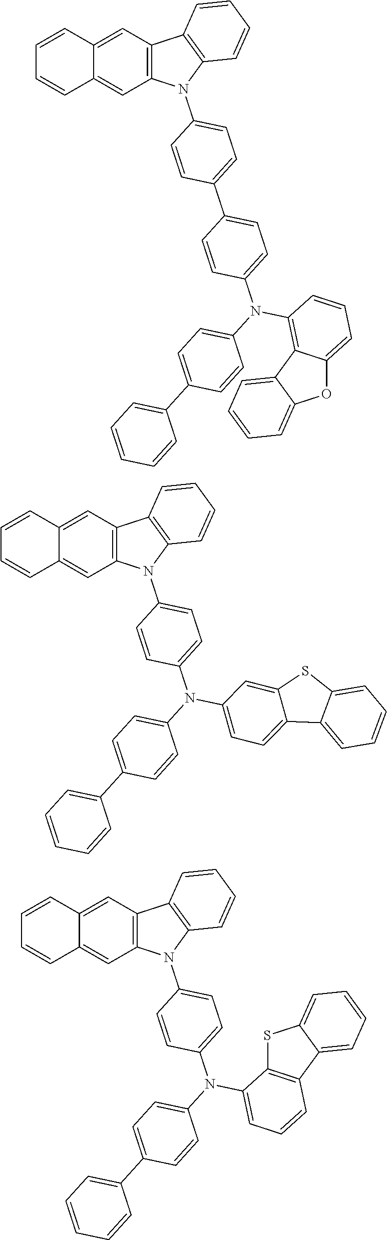

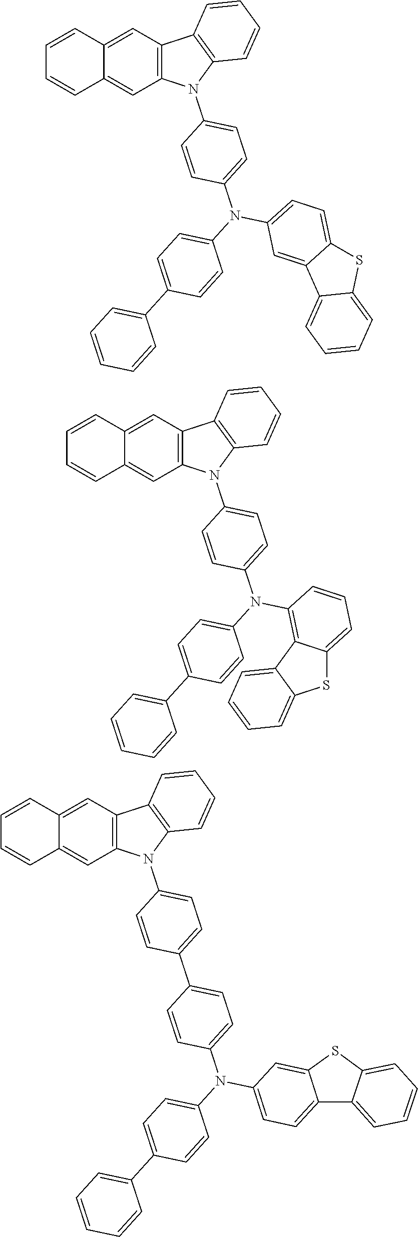

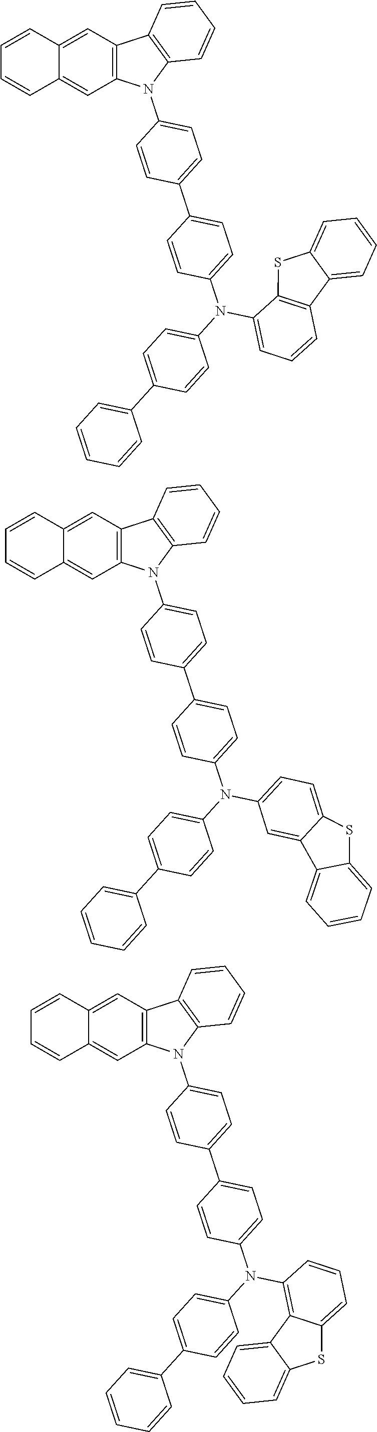

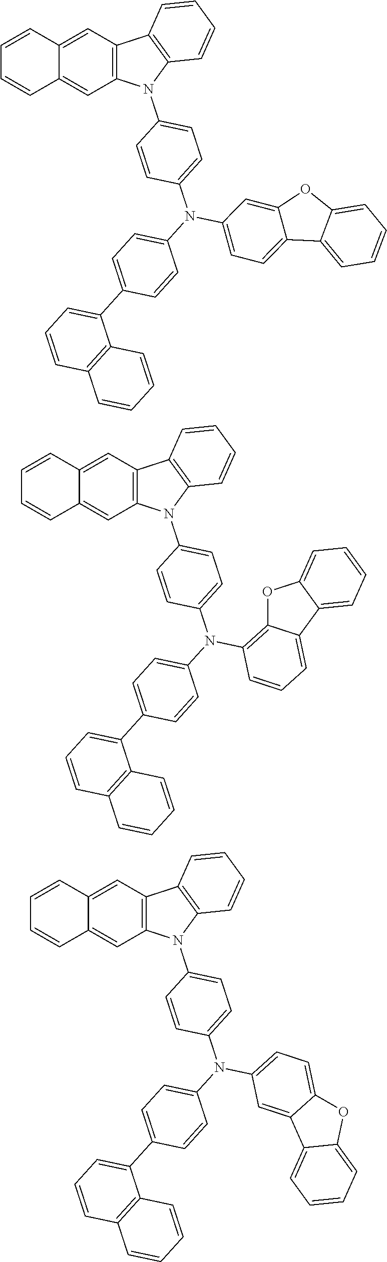

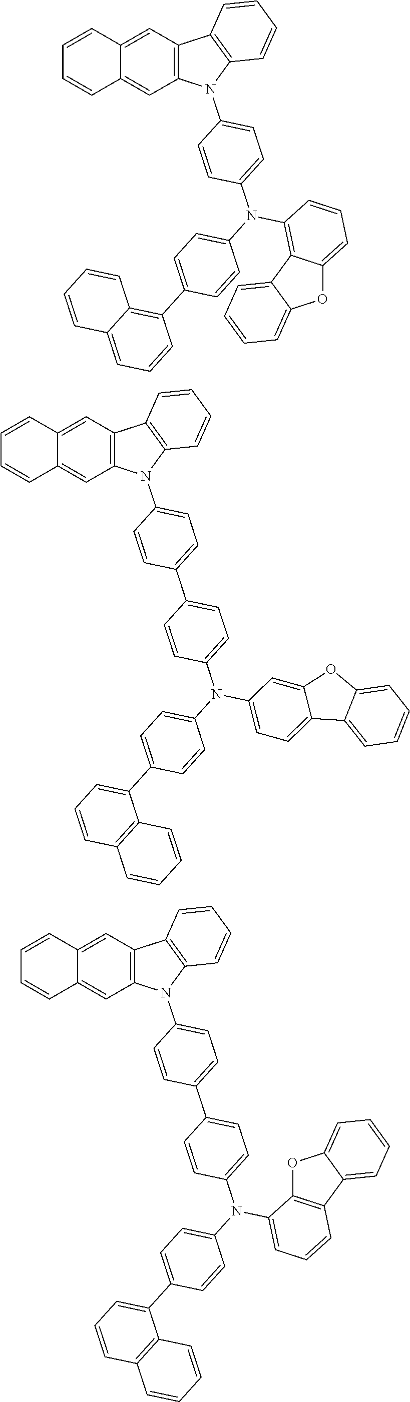

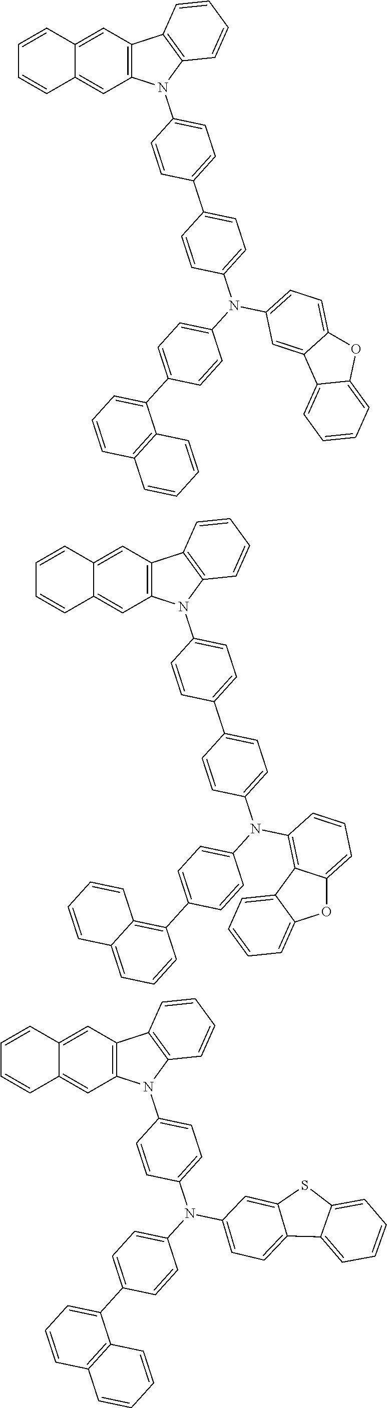

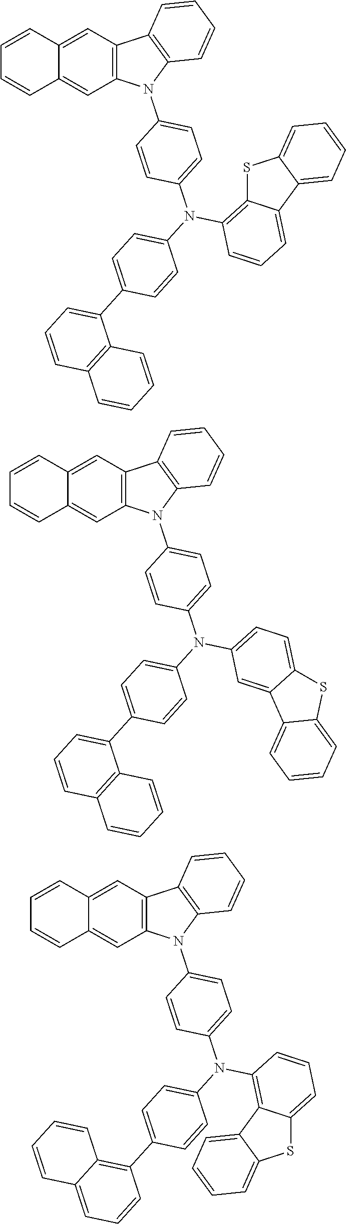

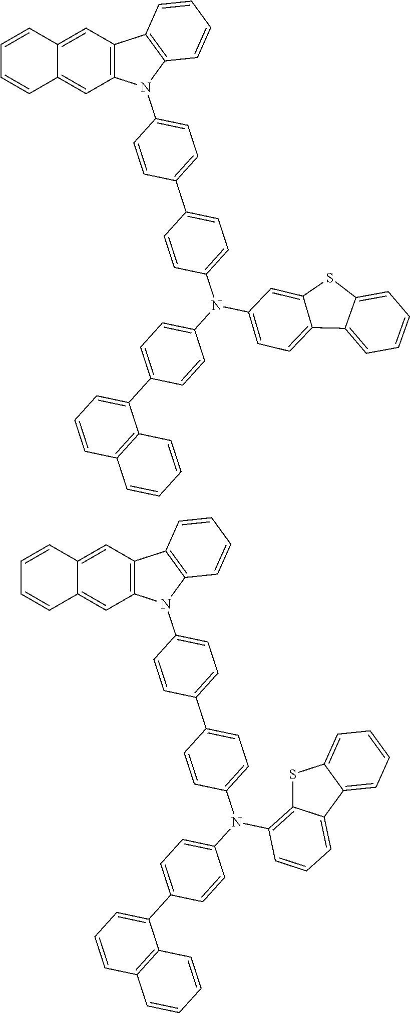

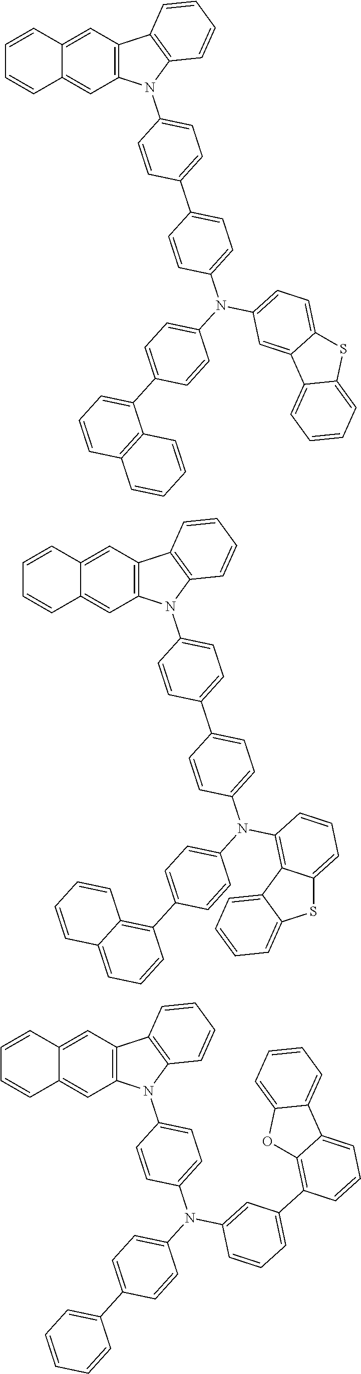

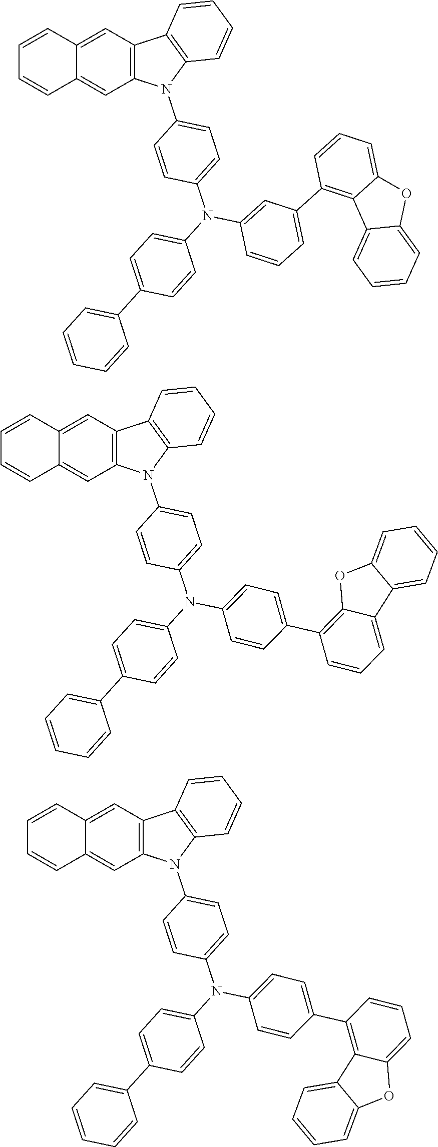 [SMILES: c1ccc(-c2ccc(N(c3ccc(-n4c5ccccc5c5cc6ccccc6cc54)cc3)c3cccc(-c4cccc5oc6ccccc6c45)c3)cc2)cc1 ; c1ccc(-c2ccc(N(c3ccc(-c4cccc5c4oc4ccccc45)cc3)c3ccc(-n4c5ccccc5c5cc6ccccc6cc54)cc3)cc2)cc1 ; c1ccc(-c2ccc(N(c3ccc(-c4cccc5oc6ccccc6c45)cc3)c3ccc(-n4c5ccccc5c5cc6ccccc6cc54)cc3)cc2)cc1]

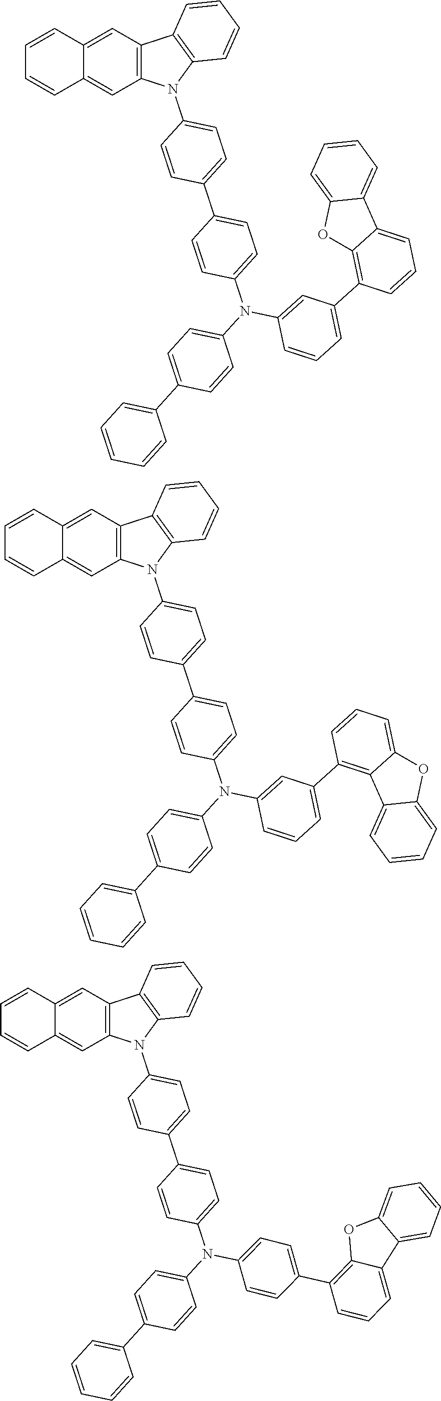

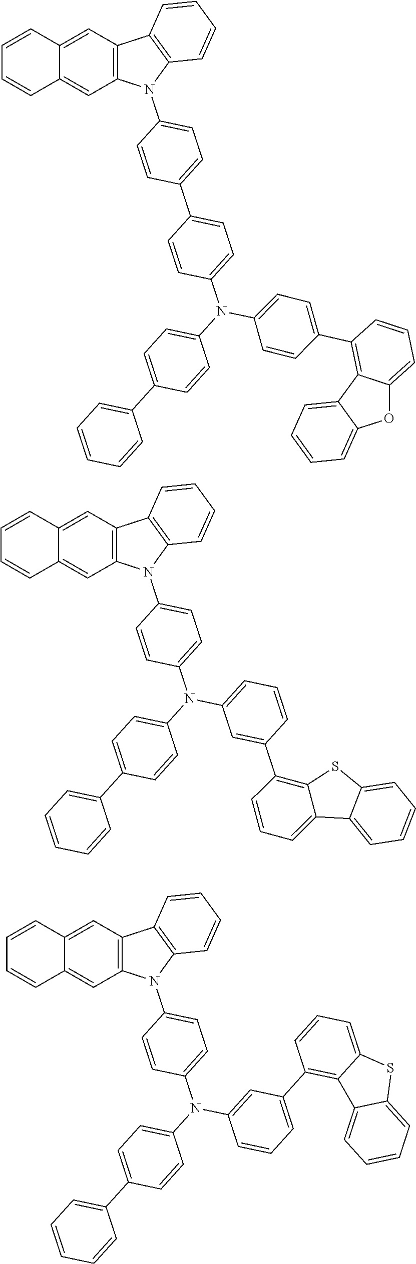

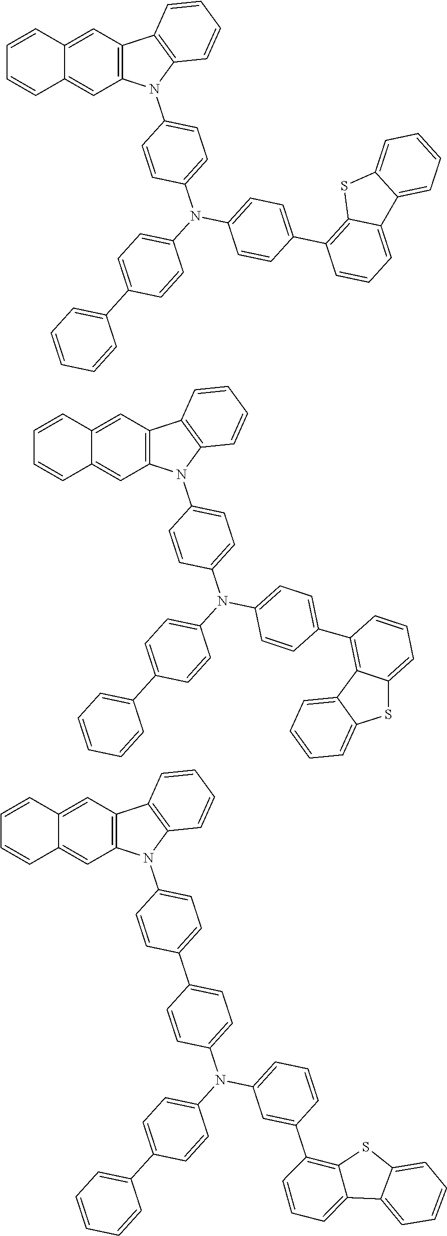

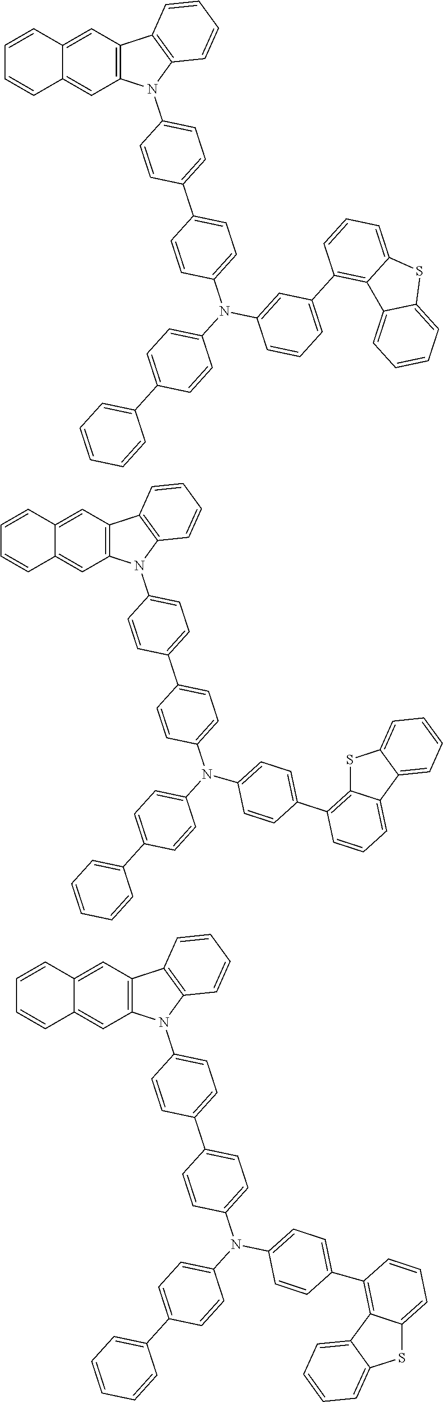

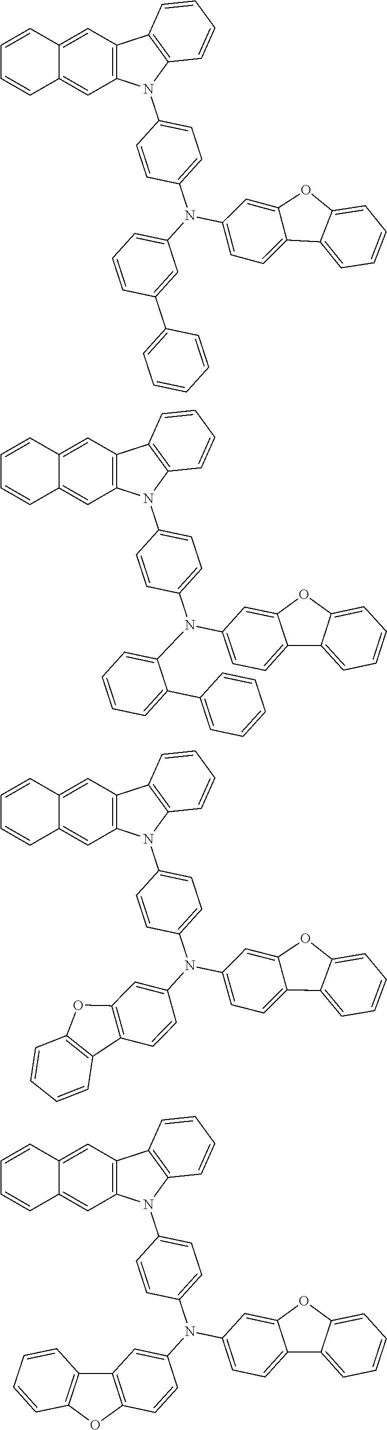

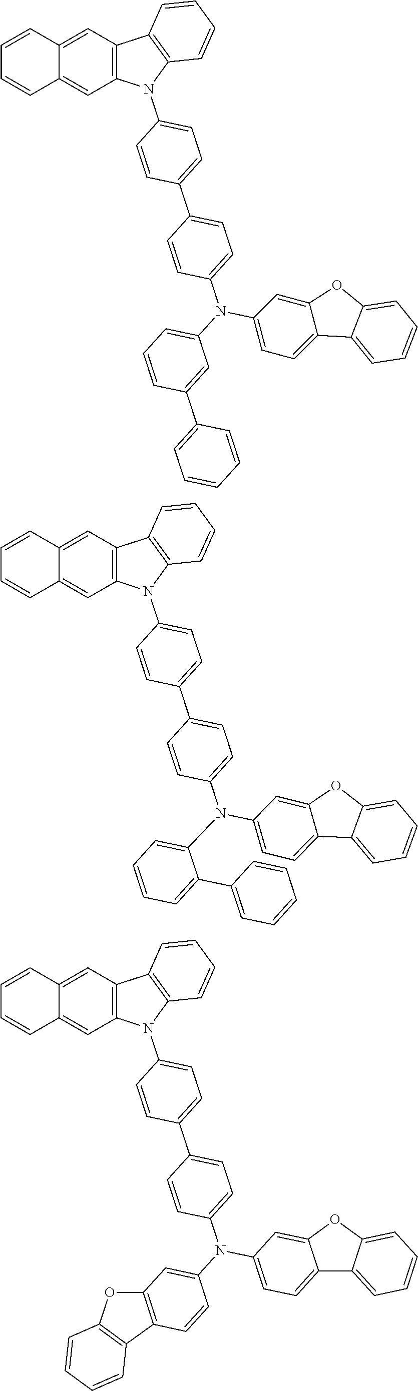

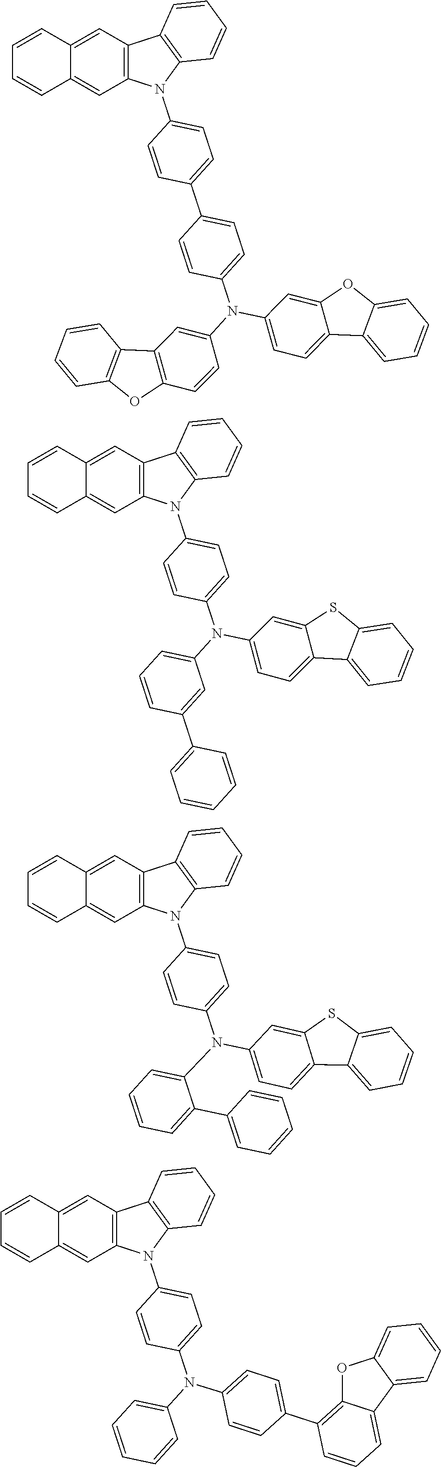

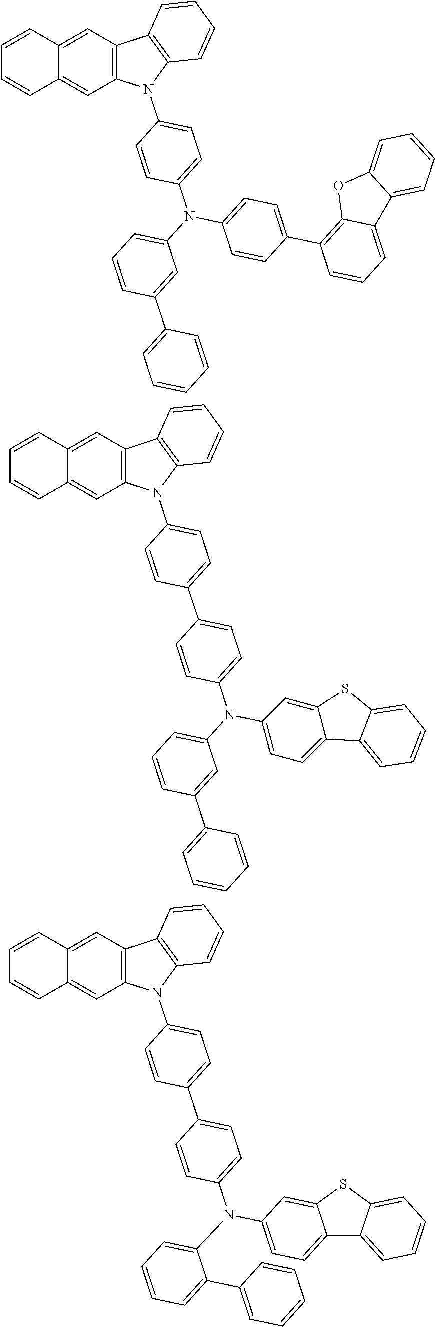

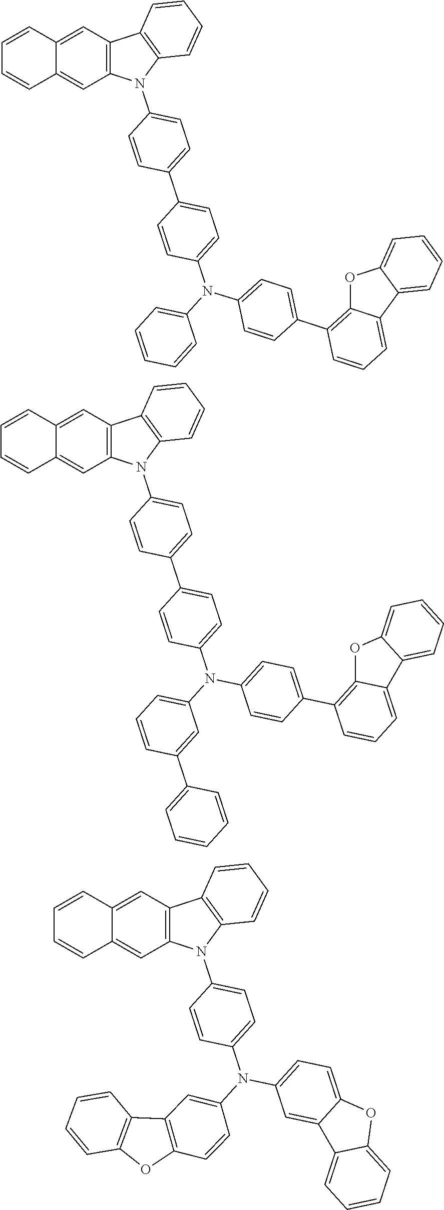

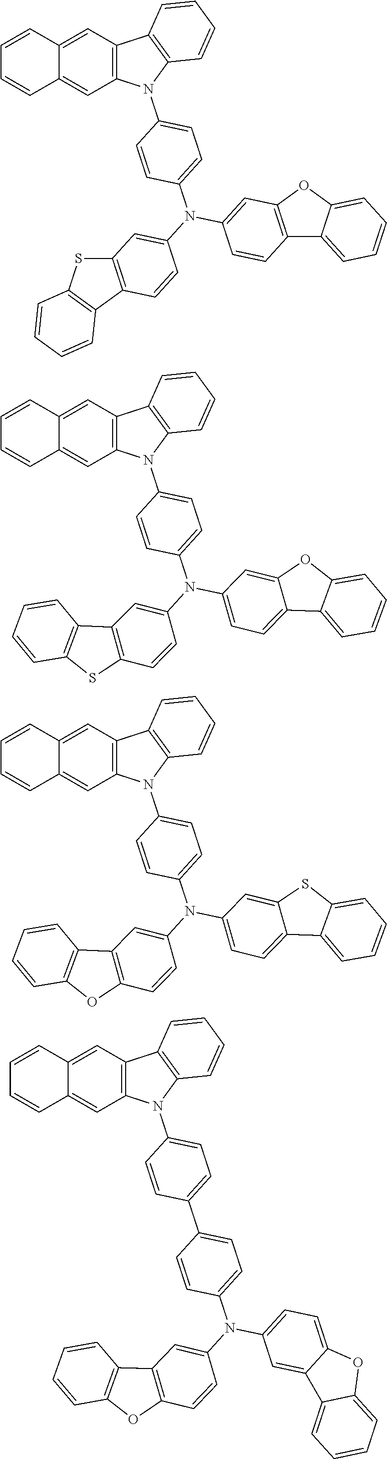

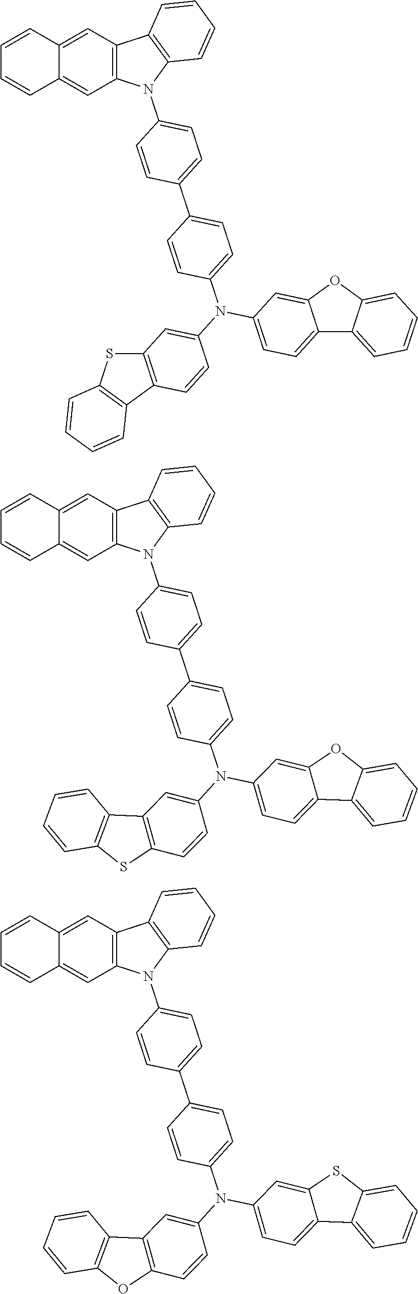

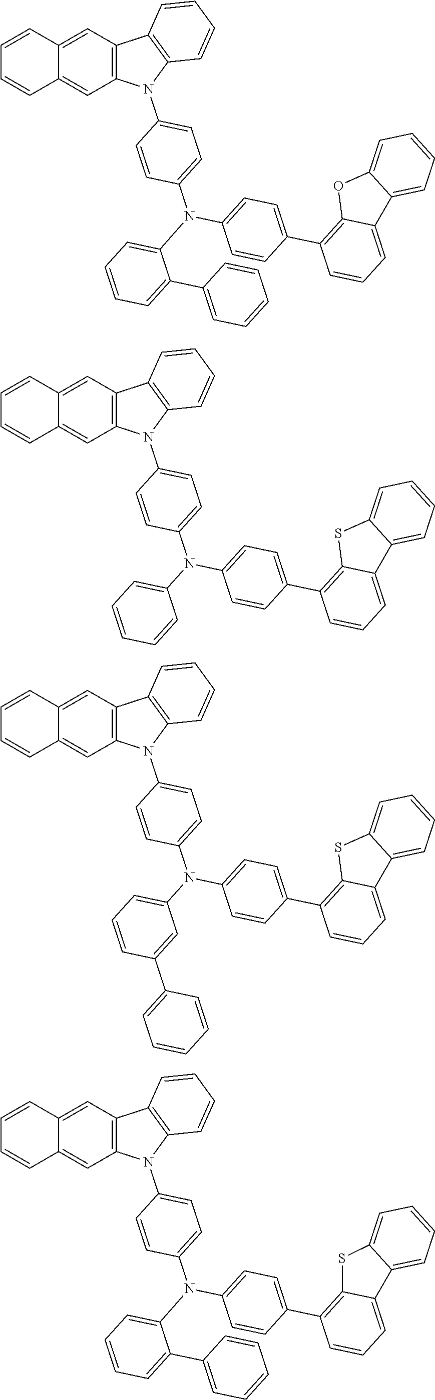

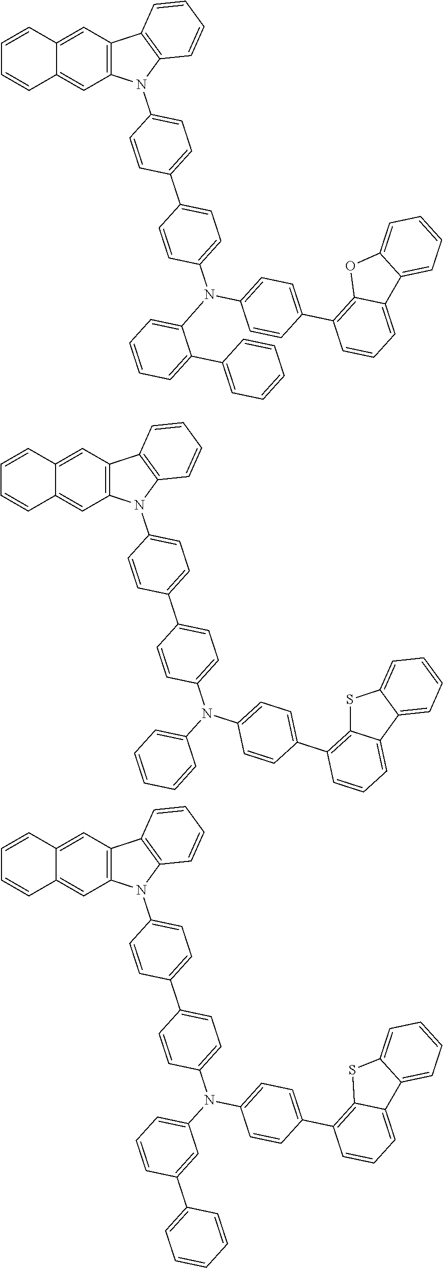

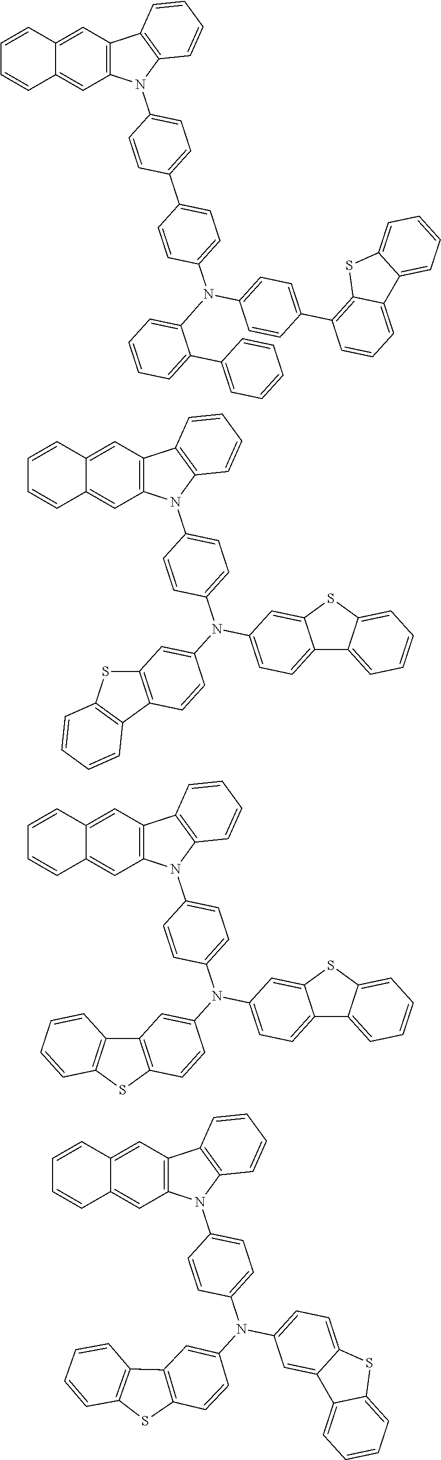

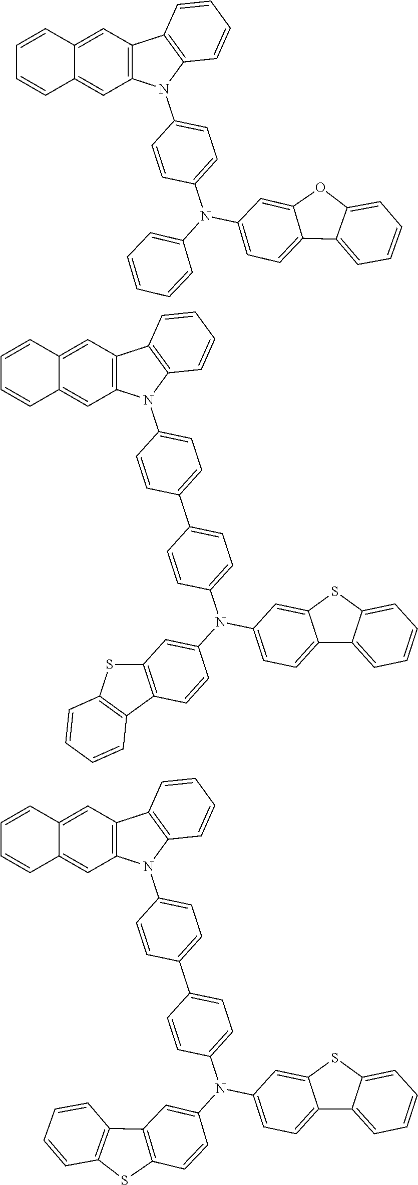

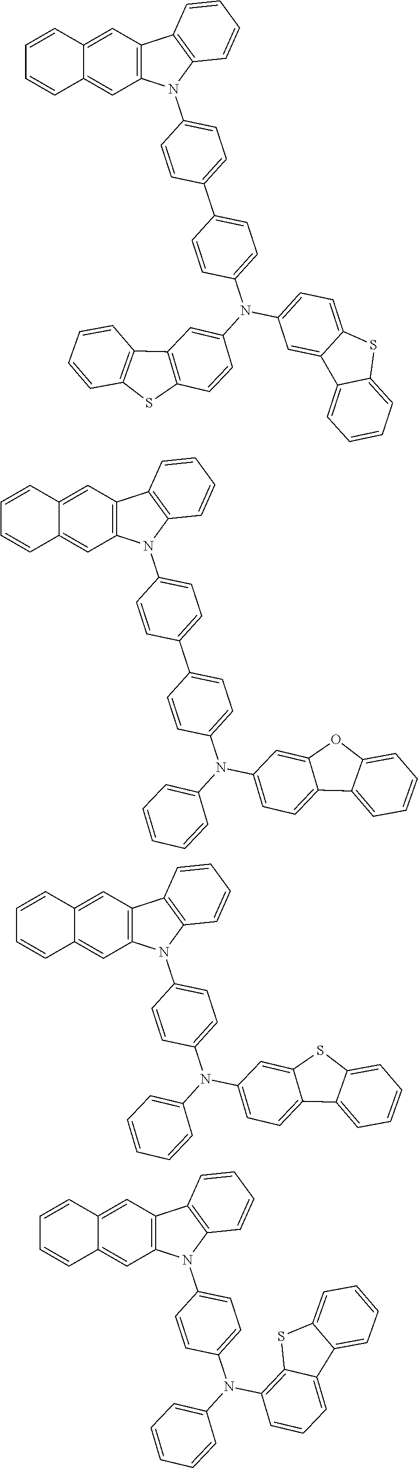

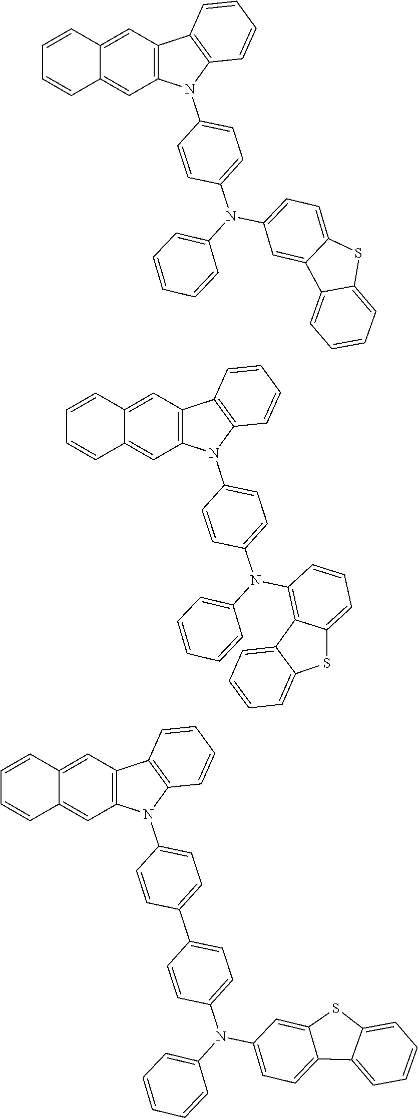 [SMILES: c1ccc(N(c2ccc(-n3c4ccccc4c4cc5ccccc5cc43)cc2)c2ccc3sc4ccccc4c3c2)cc1 ; c1ccc(N(c2ccc(-n3c4ccccc4c4cc5ccccc5cc43)cc2)c2cccc3sc4ccccc4c23)cc1 ; c1ccc(N(c2ccc(-c3ccc(-n4c5ccccc5c5cc6ccccc6cc54)cc3)cc2)c2ccc3c(c2)sc2ccccc23)cc1]

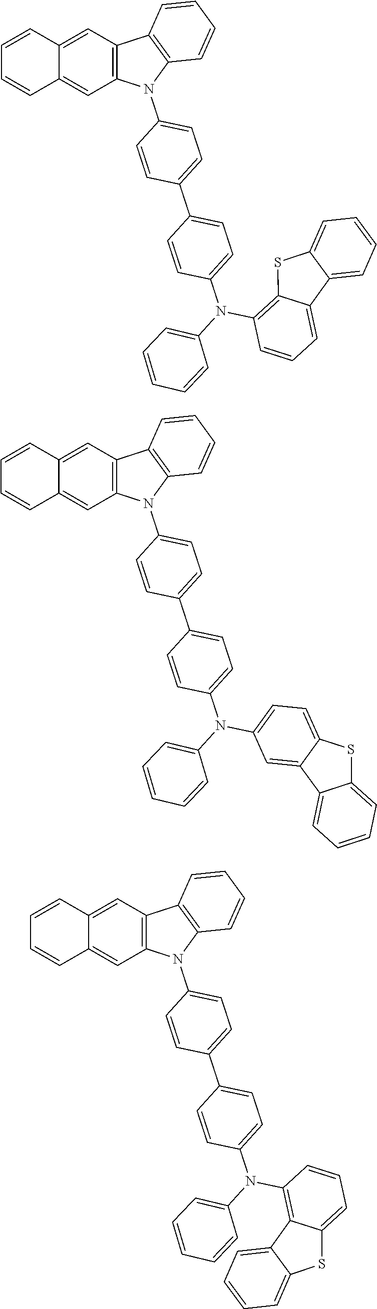

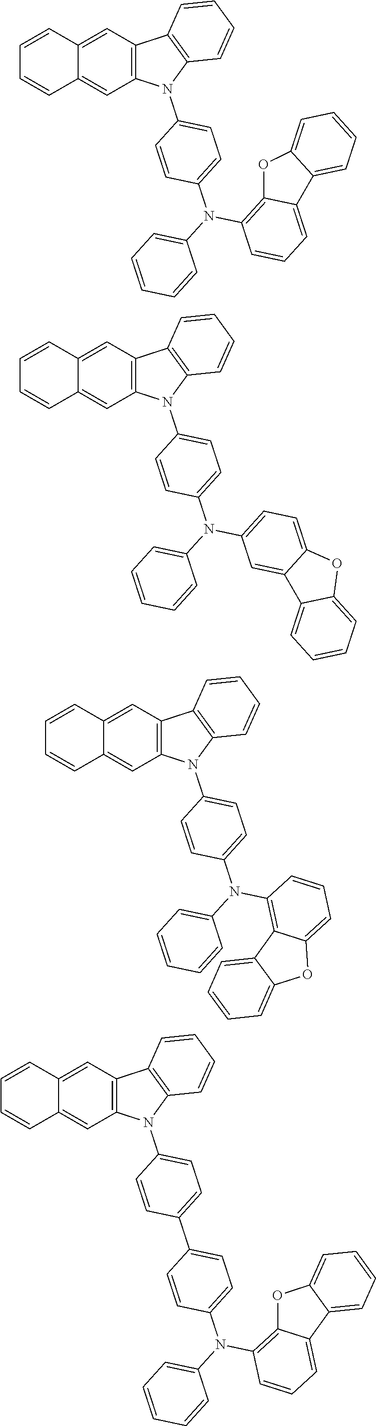

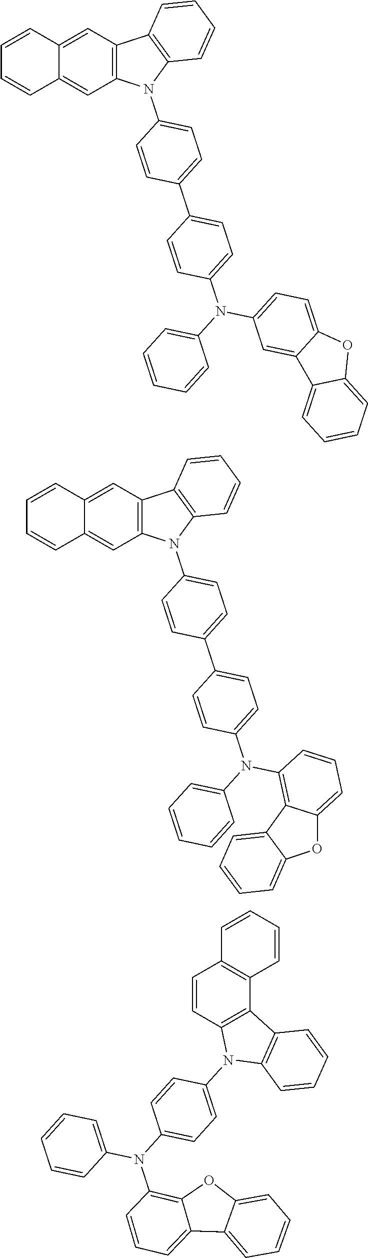

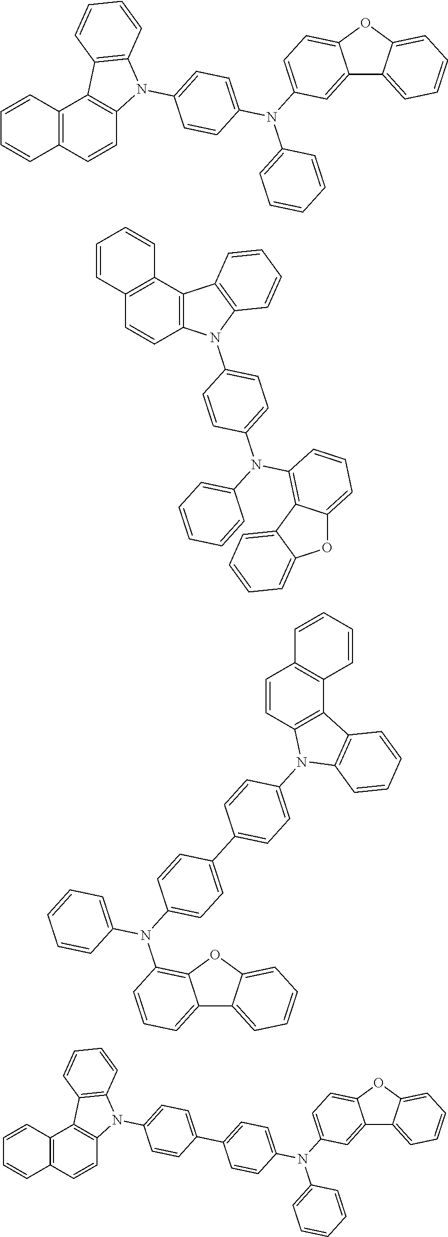

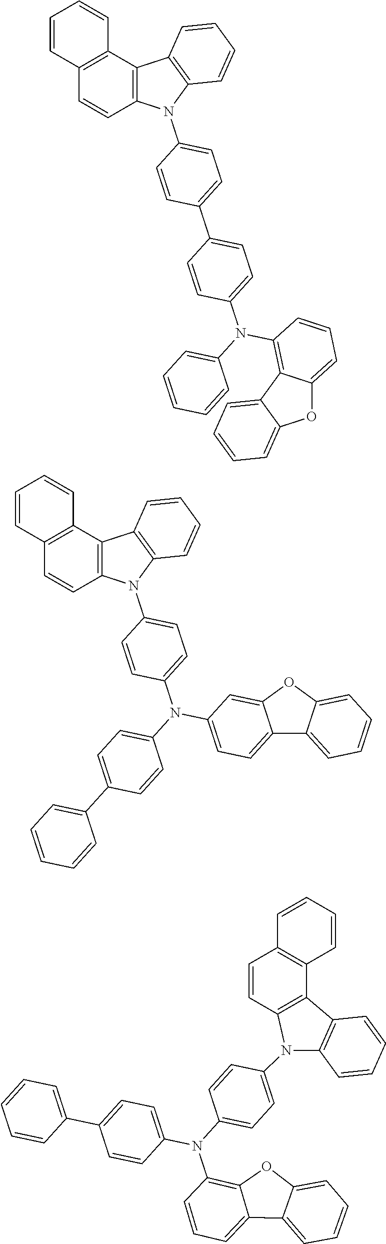 [SMILES: c1ccc(N(c2ccc(-c3ccc(-n4c5ccccc5c5c6ccccc6ccc54)cc3)cc2)c2cccc3oc4ccccc4c23)cc1 ; c1ccc(-c2ccc(N(c3ccc(-n4c5ccccc5c5c6ccccc6ccc54)cc3)c3ccc4c(c3)oc3ccccc34)cc2)cc1 ; c1ccc(-c2ccc(N(c3ccc(-n4c5ccccc5c5c6ccccc6ccc54)cc3)c3cccc4c3oc3ccccc34)cc2)cc1]

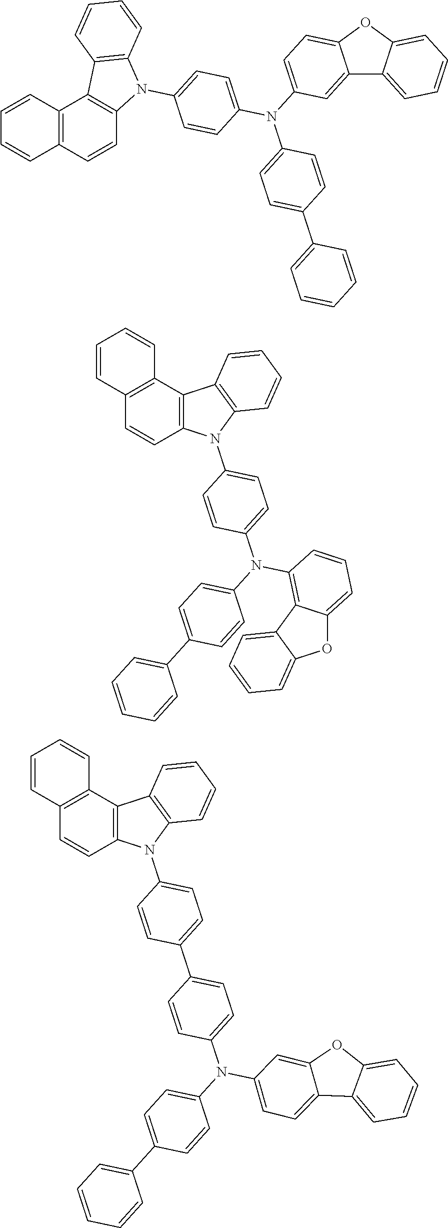

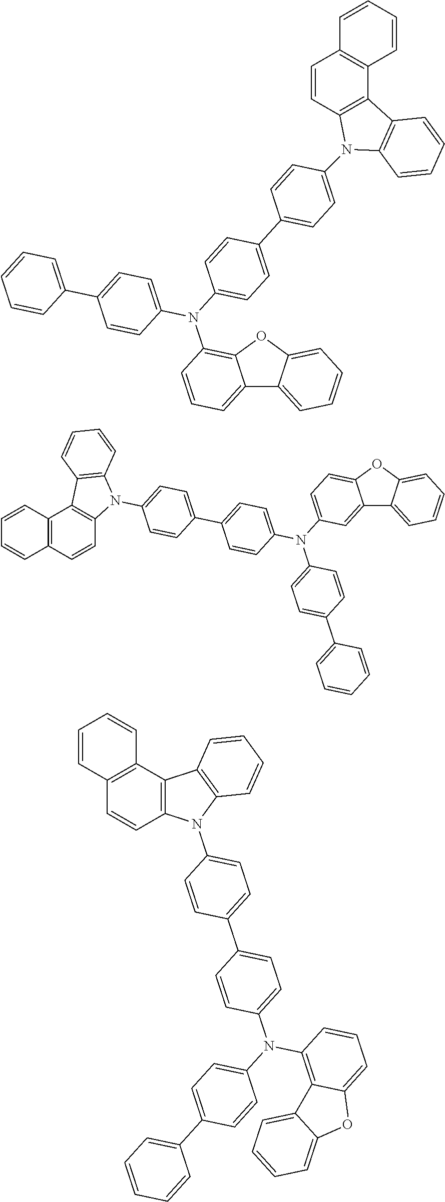 [SMILES: c1ccc(-c2ccc(N(c3ccc(-c4ccc(-n5c6ccccc6c6c7ccccc7ccc65)cc4)cc3)c3cccc4c3oc3ccccc34)cc2)cc1 ; c1ccc(-c2ccc(N(c3ccc(-c4ccc(-n5c6ccccc6c6c7ccccc7ccc65)cc4)cc3)c3ccc4oc5ccccc5c4c3)cc2)cc1 ; c1ccc(-c2ccc(N(c3ccc(-c4ccc(-n5c6ccccc6c6c7ccccc7ccc65)cc4)cc3)c3cccc4oc5ccccc5c34)cc2)cc1]

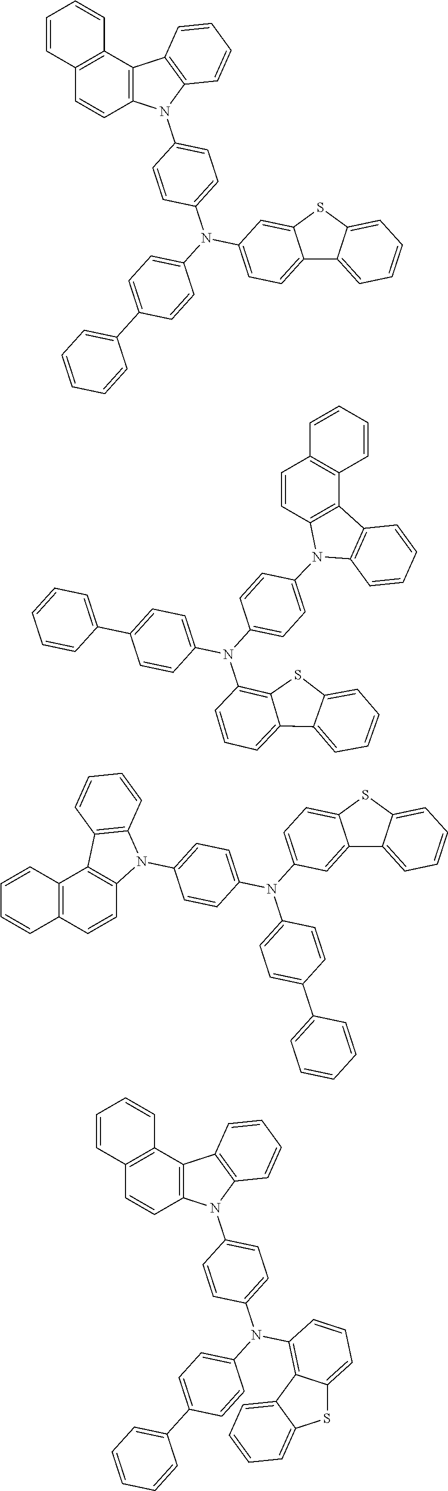 [SMILES: c1ccc(-c2ccc(N(c3ccc(-n4c5ccccc5c5c6ccccc6ccc54)cc3)c3ccc4c(c3)sc3ccccc34)cc2)cc1 ; c1ccc(-c2ccc(N(c3ccc(-n4c5ccccc5c5c6ccccc6ccc54)cc3)c3cccc4c3sc3ccccc34)cc2)cc1 ; c1ccc(-c2ccc(N(c3ccc(-n4c5ccccc5c5c6ccccc6ccc54)cc3)c3ccc4sc5ccccc5c4c3)cc2)cc1 ; c1ccc(-c2ccc(N(c3ccc(-n4c5ccccc5c5c6ccccc6ccc54)cc3)c3cccc4sc5ccccc5c34)cc2)cc1]

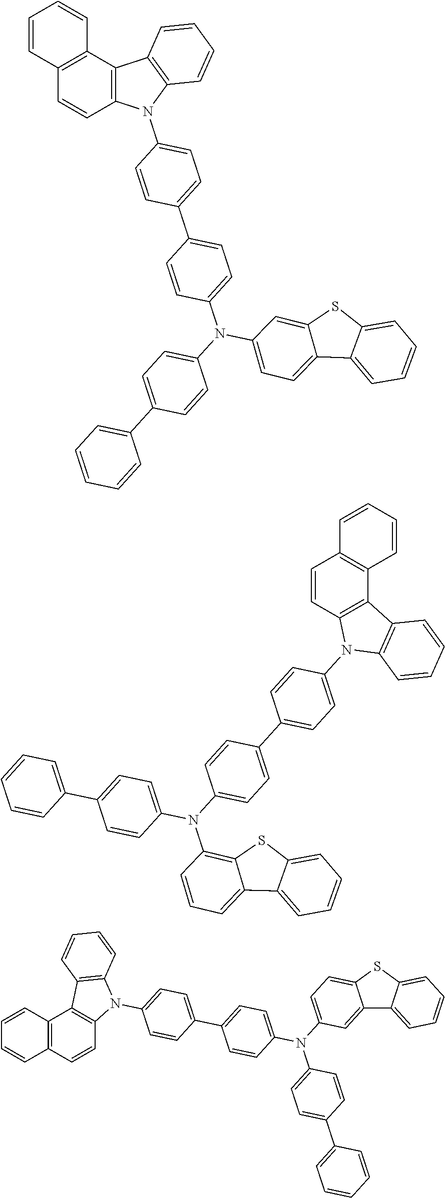 [SMILES: c1ccc(-c2ccc(N(c3ccc(-c4ccc(-n5c6ccccc6c6c7ccccc7ccc65)cc4)cc3)c3ccc4c(c3)sc3ccccc34)cc2)cc1 ; c1ccc(-c2ccc(N(c3ccc(-c4ccc(-n5c6ccccc6c6c7ccccc7ccc65)cc4)cc3)c3cccc4c3sc3ccccc34)cc2)cc1 ; c1ccc(-c2ccc(N(c3ccc(-c4ccc(-n5c6ccccc6c6c7ccccc7ccc65)cc4)cc3)c3ccc4sc5ccccc5c4c3)cc2)cc1]

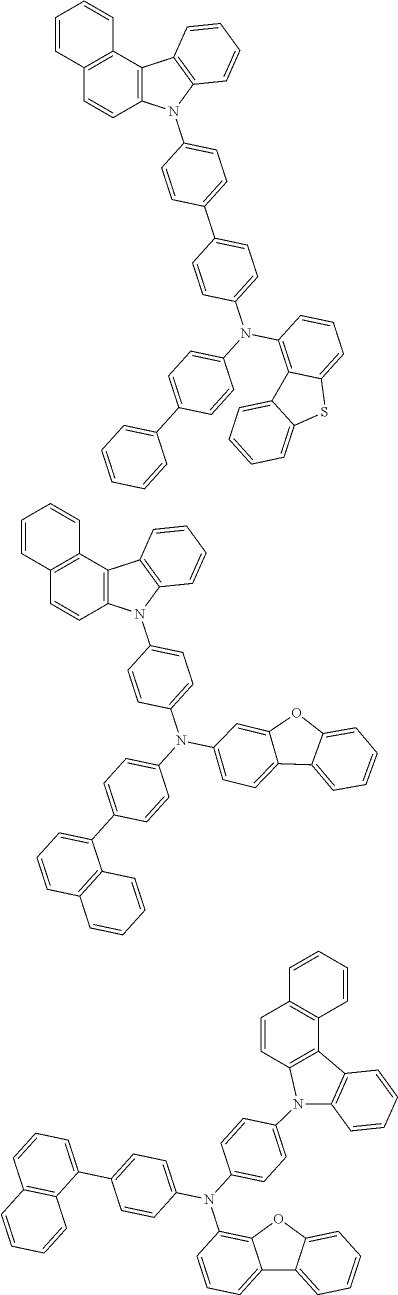

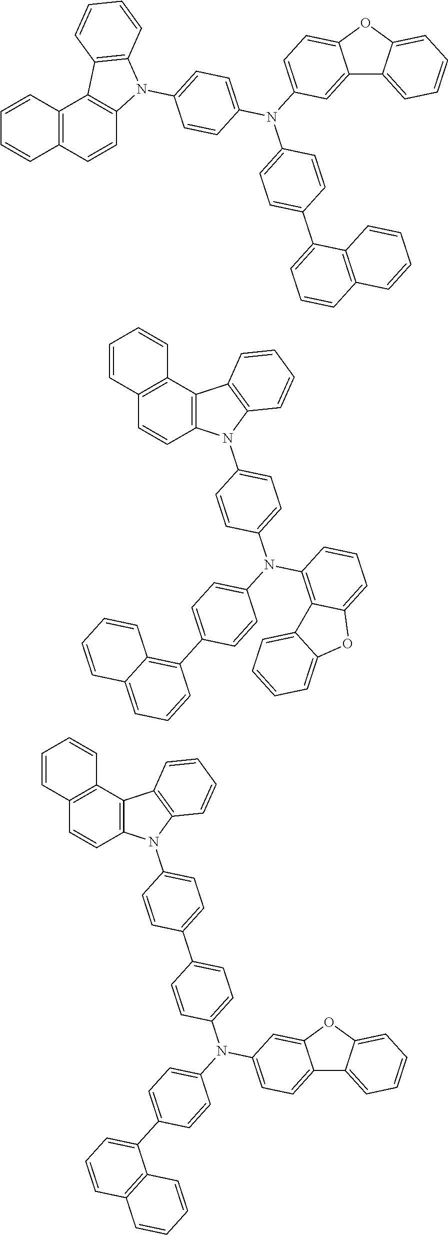

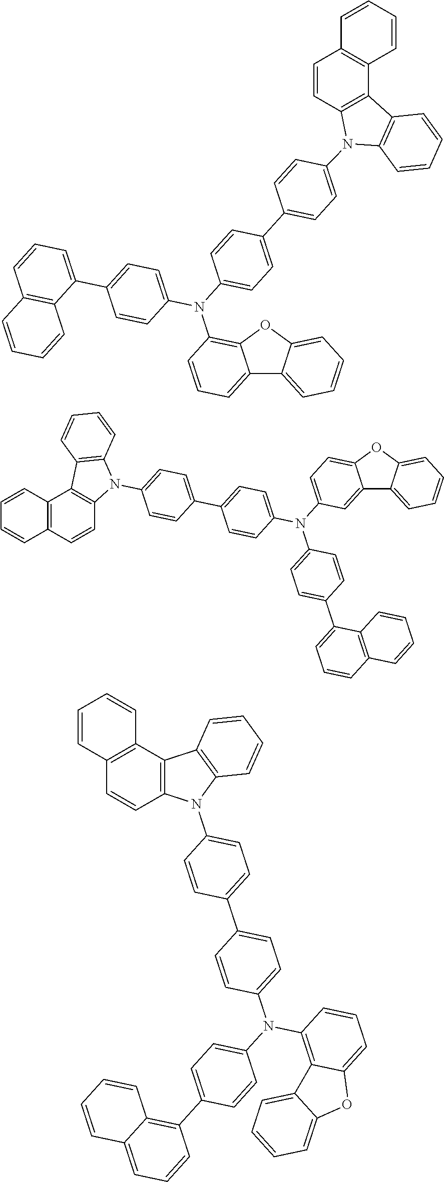 [SMILES: c1ccc2c(-c3ccc(N(c4ccc(-c5ccc(-n6c7ccccc7c7c8ccccc8ccc76)cc5)cc4)c4cccc5c4oc4ccccc45)cc3)cccc2c1 ; c1ccc2c(-c3ccc(N(c4ccc(-c5ccc(-n6c7ccccc7c7c8ccccc8ccc76)cc5)cc4)c4ccc5oc6ccccc6c5c4)cc3)cccc2c1 ; c1ccc2c(-c3ccc(N(c4ccc(-c5ccc(-n6c7ccccc7c7c8ccccc8ccc76)cc5)cc4)c4cccc5oc6ccccc6c45)cc3)cccc2c1]

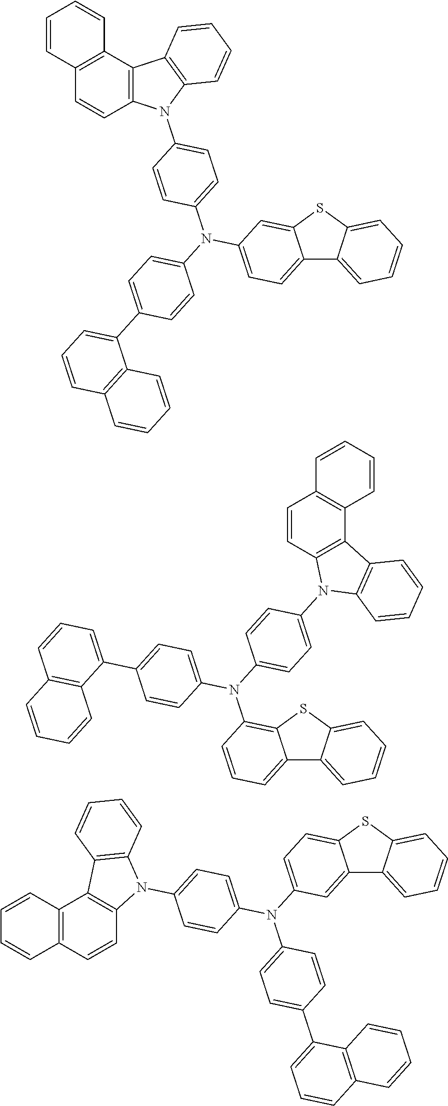

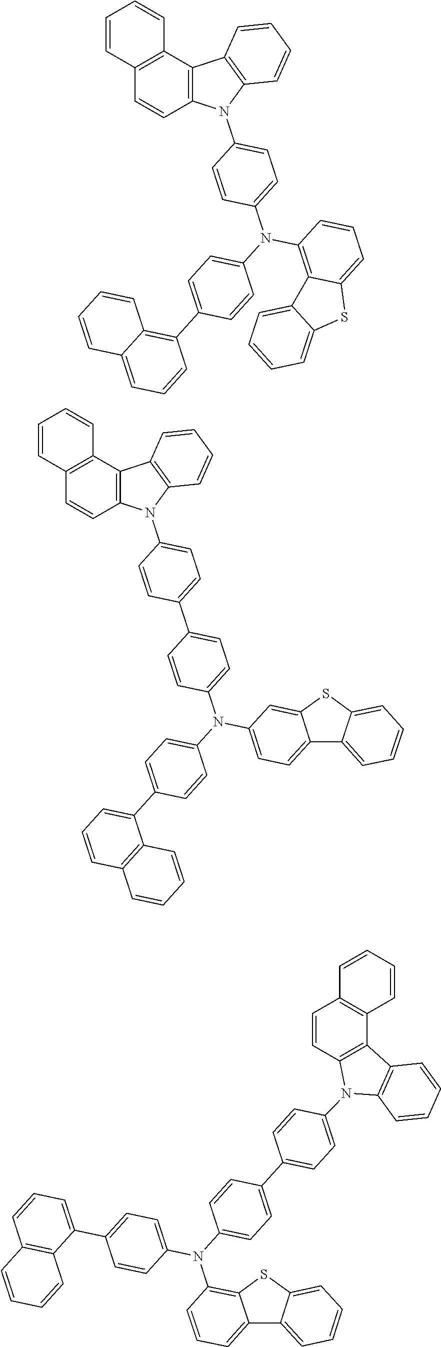

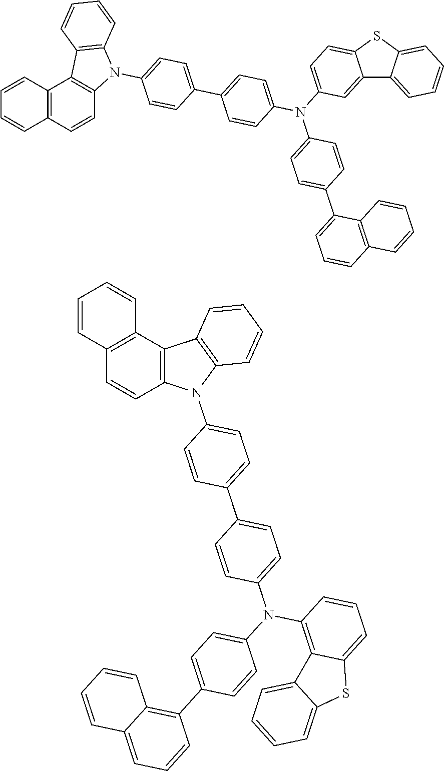 [SMILES: c1ccc2c(-c3ccc(N(c4ccc(-c5ccc(-n6c7ccccc7c7c8ccccc8ccc76)cc5)cc4)c4ccc5sc6ccccc6c5c4)cc3)cccc2c1 ; c1ccc2c(-c3ccc(N(c4ccc(-c5ccc(-n6c7ccccc7c7c8ccccc8ccc76)cc5)cc4)c4cccc5sc6ccccc6c45)cc3)cccc2c1]

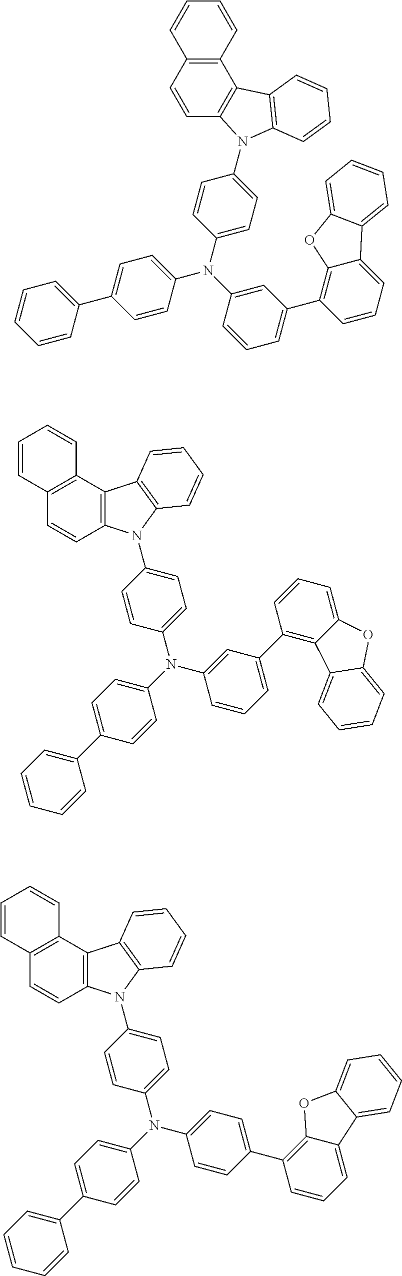 [SMILES: c1ccc(-c2ccc(N(c3ccc(-n4c5ccccc5c5c6ccccc6ccc54)cc3)c3cccc(-c4cccc5c4oc4ccccc45)c3)cc2)cc1 ; c1ccc(-c2ccc(N(c3ccc(-n4c5ccccc5c5c6ccccc6ccc54)cc3)c3cccc(-c4cccc5oc6ccccc6c45)c3)cc2)cc1 ; c1ccc(-c2ccc(N(c3ccc(-c4cccc5c4oc4ccccc45)cc3)c3ccc(-n4c5ccccc5c5c6ccccc6ccc54)cc3)cc2)cc1]

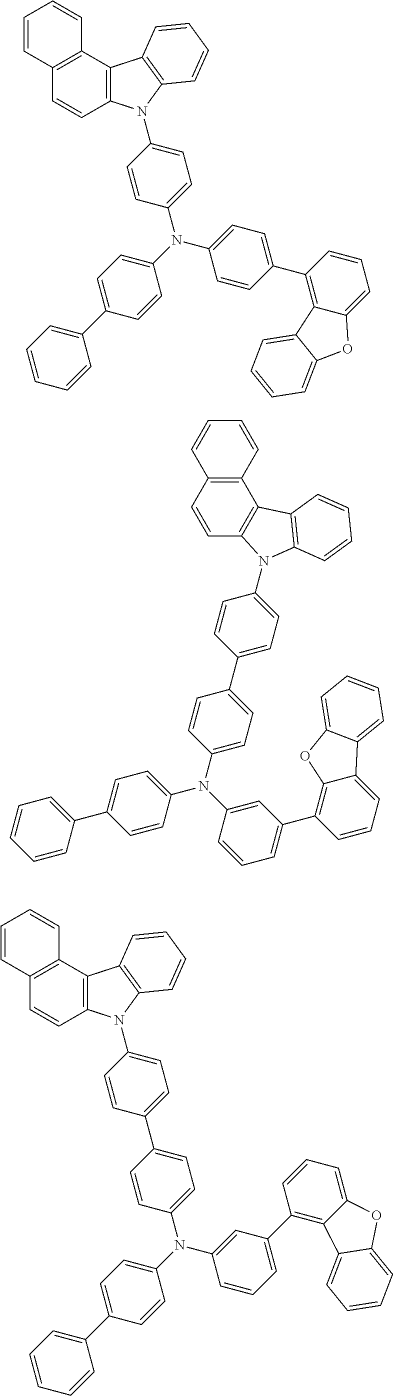

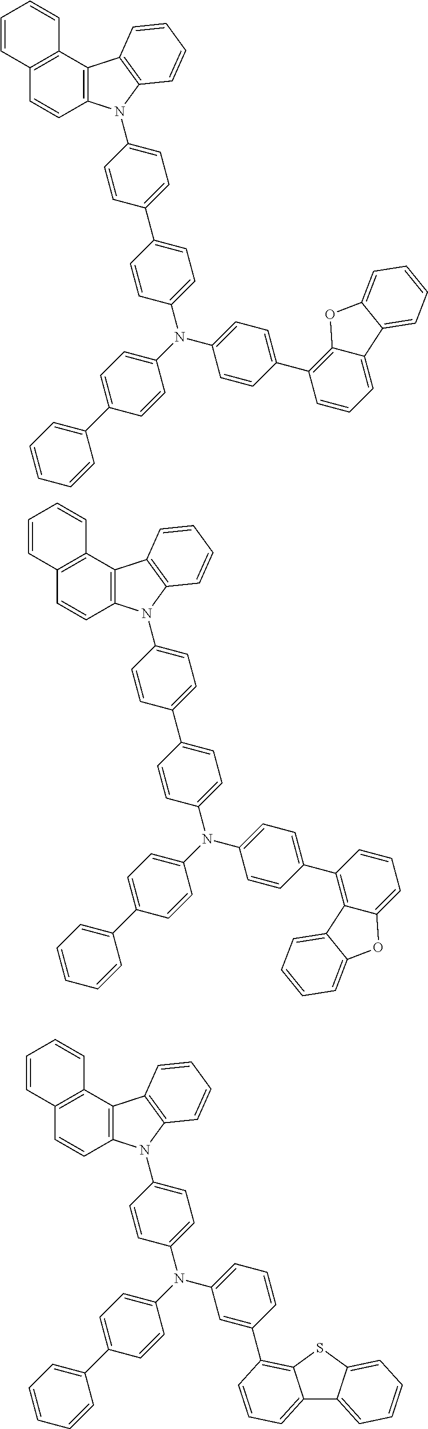

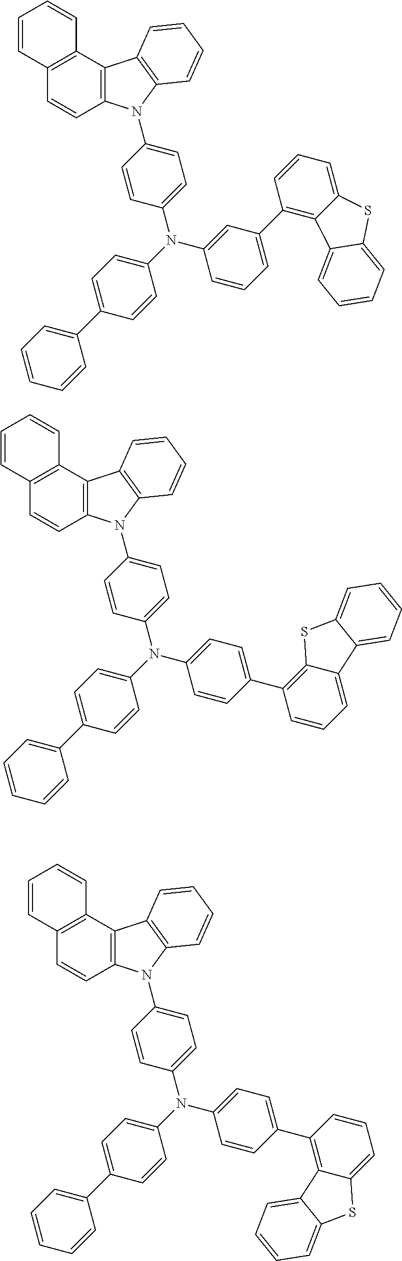

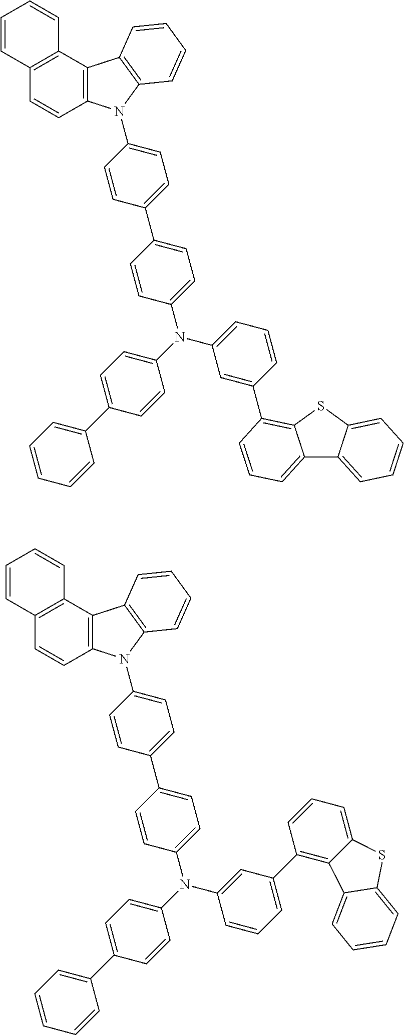 [SMILES: c1ccc(-c2ccc(N(c3ccc(-c4ccc(-n5c6ccccc6c6c7ccccc7ccc65)cc4)cc3)c3cccc(-c4cccc5c4sc4ccccc45)c3)cc2)cc1 ; c1ccc(-c2ccc(N(c3ccc(-c4ccc(-n5c6ccccc6c6c7ccccc7ccc65)cc4)cc3)c3cccc(-c4cccc5sc6ccccc6c45)c3)cc2)cc1]

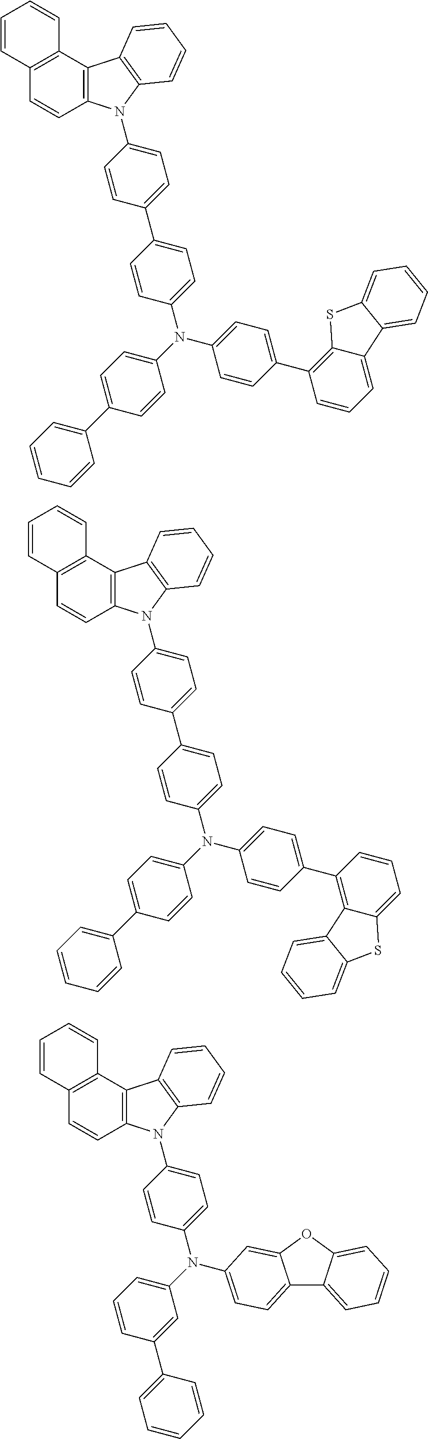

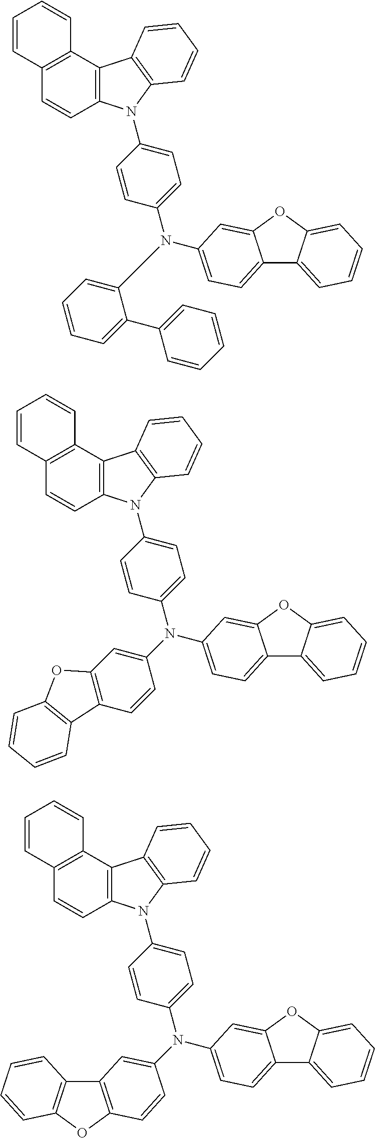

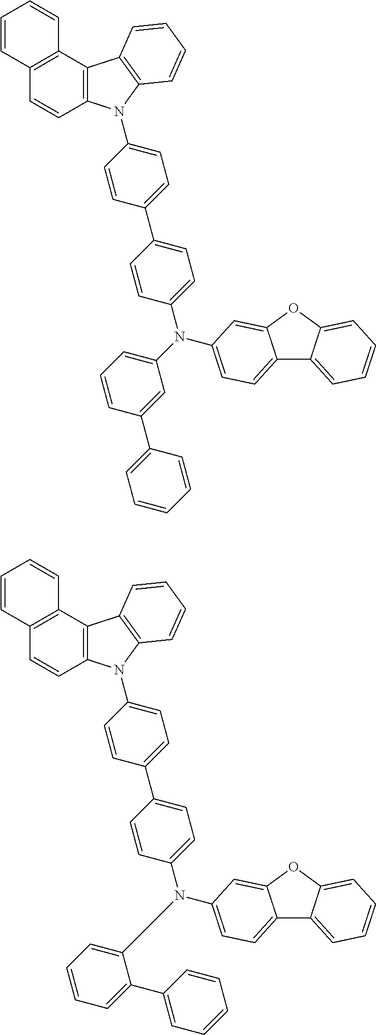 [SMILES: c1ccc(-c2cccc(N(c3ccc(-c4ccc(-n5c6ccccc6c6c7ccccc7ccc65)cc4)cc3)c3ccc4c(c3)oc3ccccc34)c2)cc1 ; c1ccc(-c2ccccc2N(c2ccc(-c3ccc(-n4c5ccccc5c5c6ccccc6ccc54)cc3)cc2)c2ccc3c(c2)oc2ccccc23)cc1]

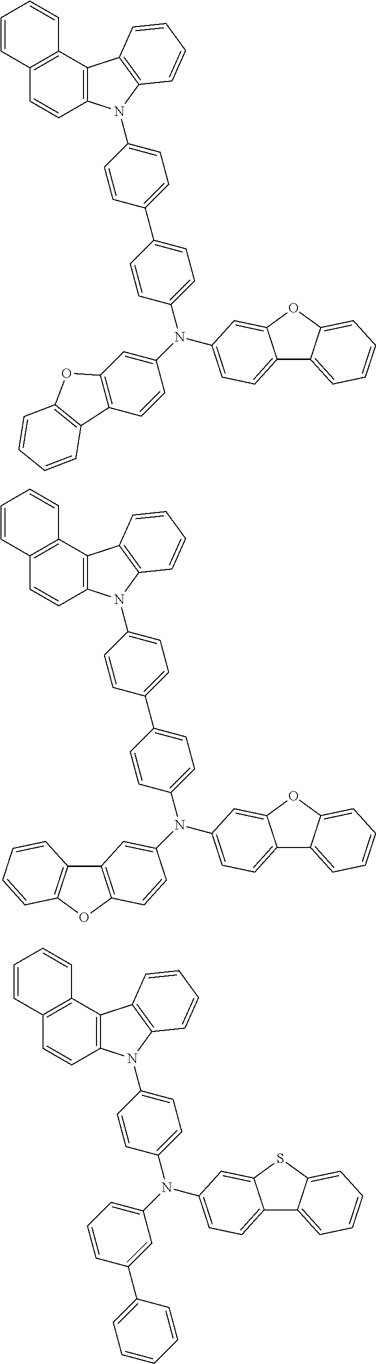

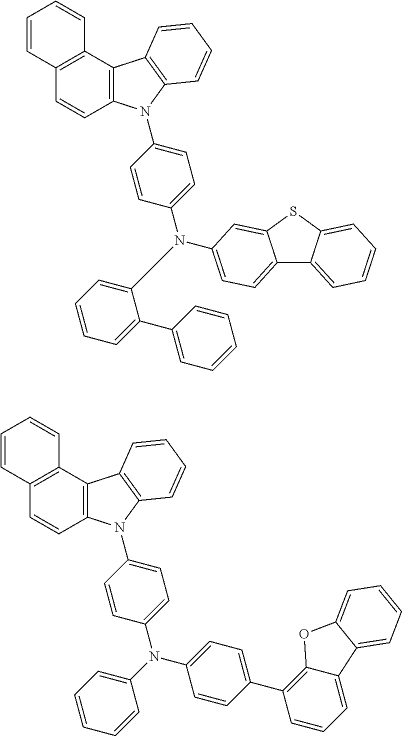 [SMILES: c1ccc(-c2ccccc2N(c2ccc(-n3c4ccccc4c4c5ccccc5ccc43)cc2)c2ccc3c(c2)sc2ccccc23)cc1 ; c1ccc(N(c2ccc(-c3cccc4c3oc3ccccc34)cc2)c2ccc(-n3c4ccccc4c4c5ccccc5ccc43)cc2)cc1]

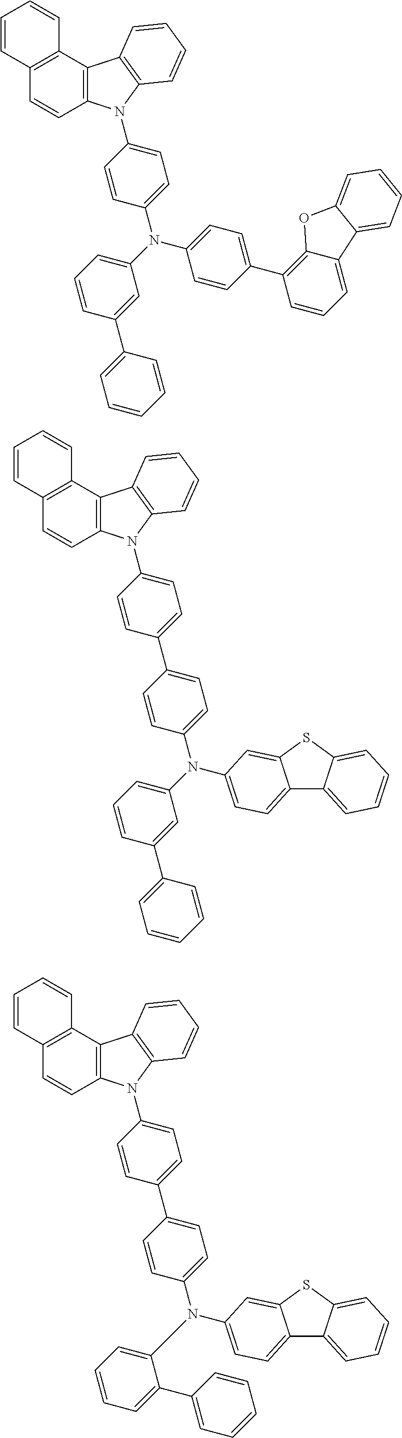

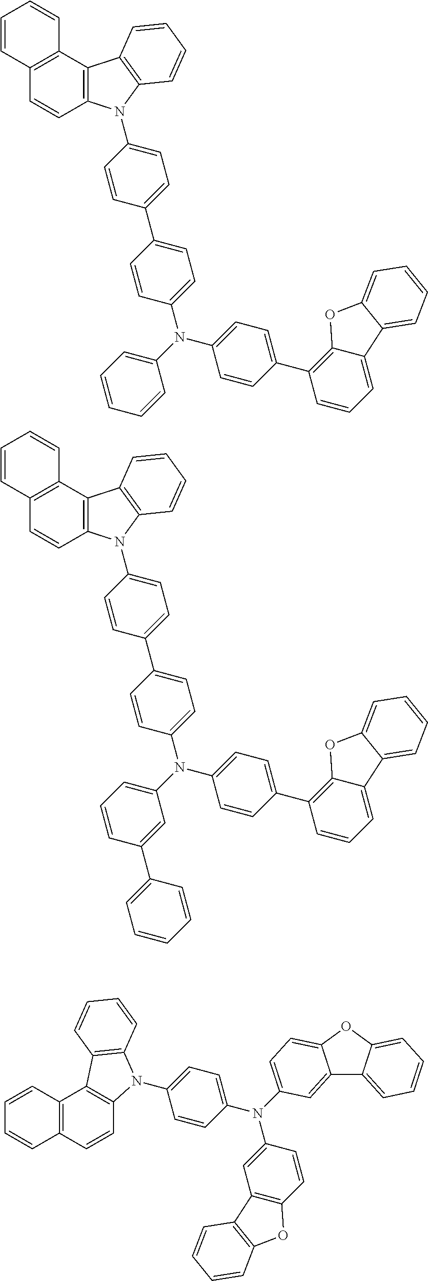

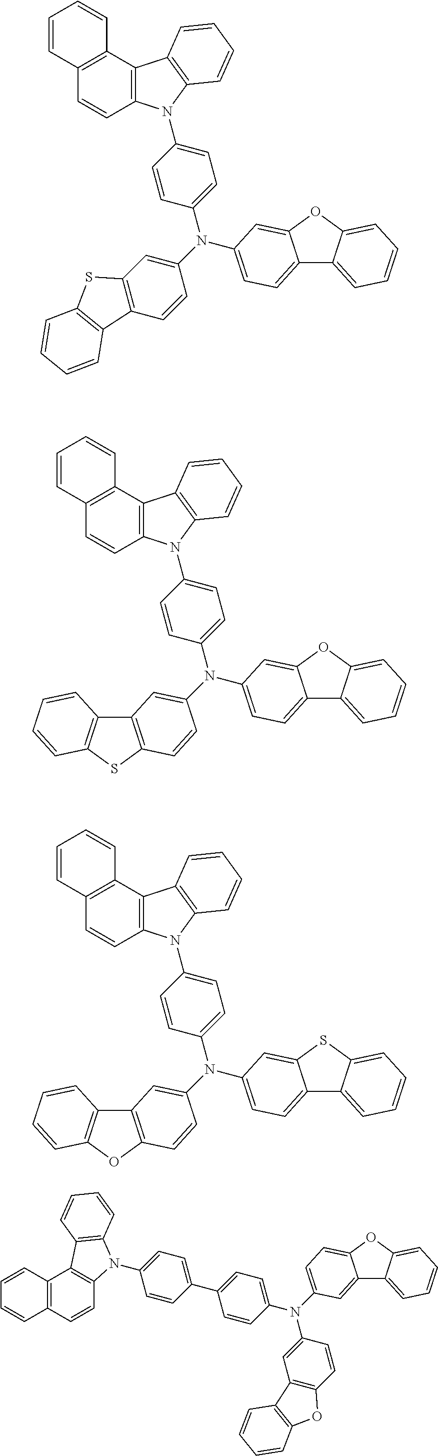

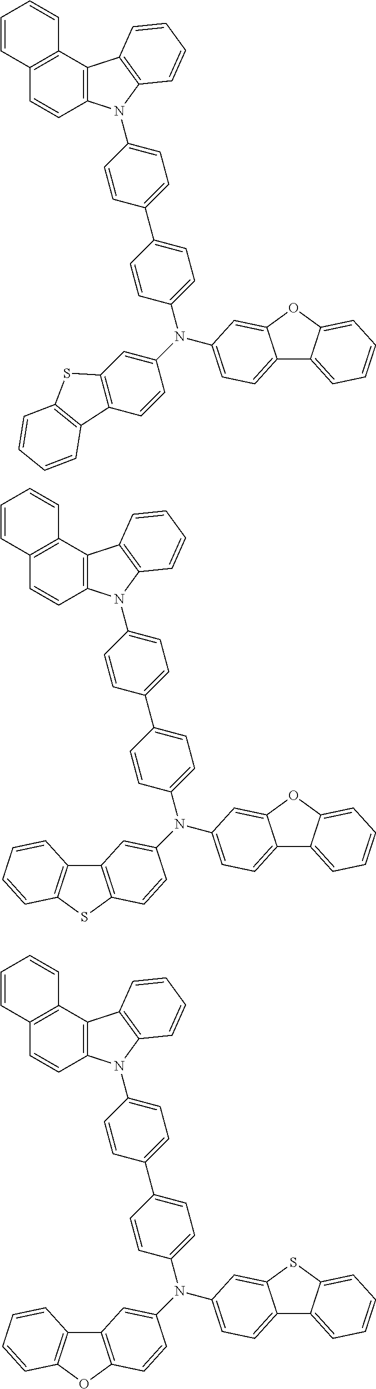

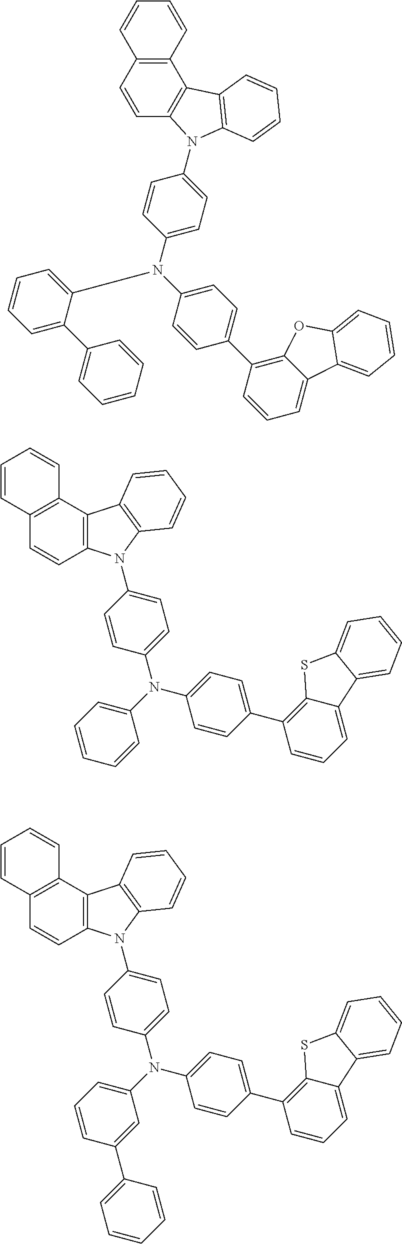 [SMILES: c1ccc(-c2ccccc2N(c2ccc(-c3cccc4c3oc3ccccc34)cc2)c2ccc(-n3c4ccccc4c4c5ccccc5ccc43)cc2)cc1 ; c1ccc(N(c2ccc(-c3cccc4c3sc3ccccc34)cc2)c2ccc(-n3c4ccccc4c4c5ccccc5ccc43)cc2)cc1 ; c1ccc(-c2cccc(N(c3ccc(-c4cccc5c4sc4ccccc45)cc3)c3ccc(-n4c5ccccc5c5c6ccccc6ccc54)cc3)c2)cc1]

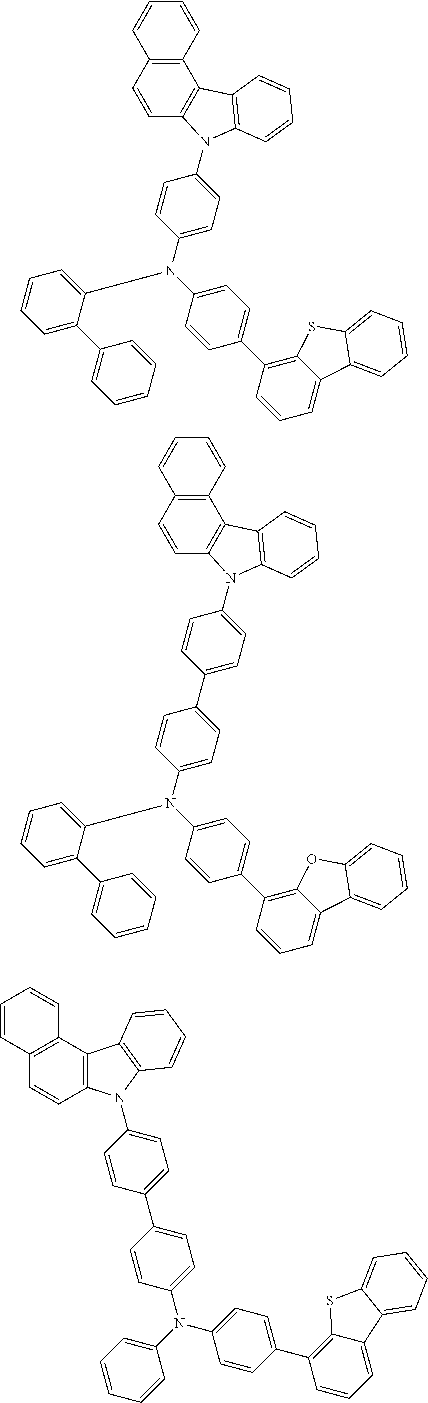 [SMILES: c1ccc(-c2ccccc2N(c2ccc(-c3cccc4c3sc3ccccc34)cc2)c2ccc(-n3c4ccccc4c4c5ccccc5ccc43)cc2)cc1 ; c1ccc(-c2ccccc2N(c2ccc(-c3ccc(-n4c5ccccc5c5c6ccccc6ccc54)cc3)cc2)c2ccc(-c3cccc4c3oc3ccccc34)cc2)cc1 ; c1ccc(N(c2ccc(-c3ccc(-n4c5ccccc5c5c6ccccc6ccc54)cc3)cc2)c2ccc(-c3cccc4c3sc3ccccc34)cc2)cc1]

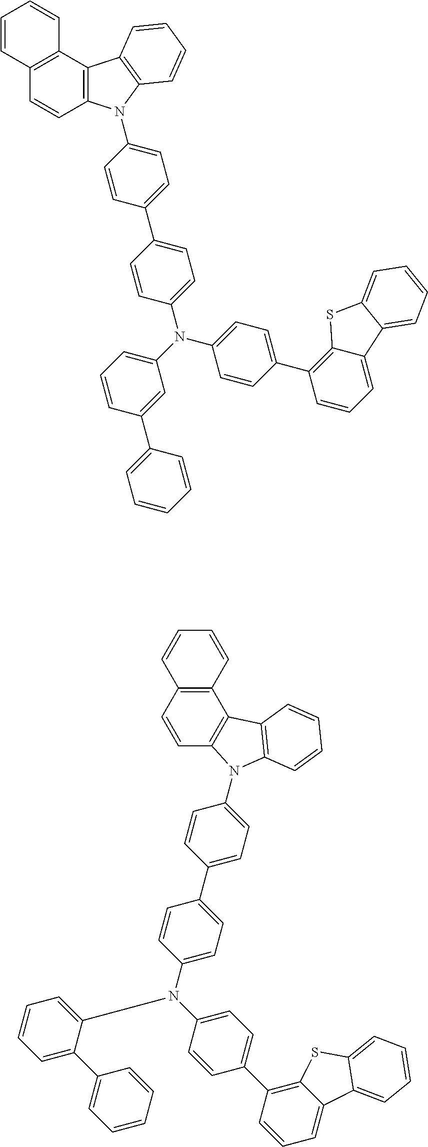 [SMILES: c1ccc(-c2cccc(N(c3ccc(-c4ccc(-n5c6ccccc6c6c7ccccc7ccc65)cc4)cc3)c3ccc(-c4cccc5c4sc4ccccc45)cc3)c2)cc1 ; c1ccc(-c2ccccc2N(c2ccc(-c3ccc(-n4c5ccccc5c5c6ccccc6ccc54)cc3)cc2)c2ccc(-c3cccc4c3sc3ccccc34)cc2)cc1]

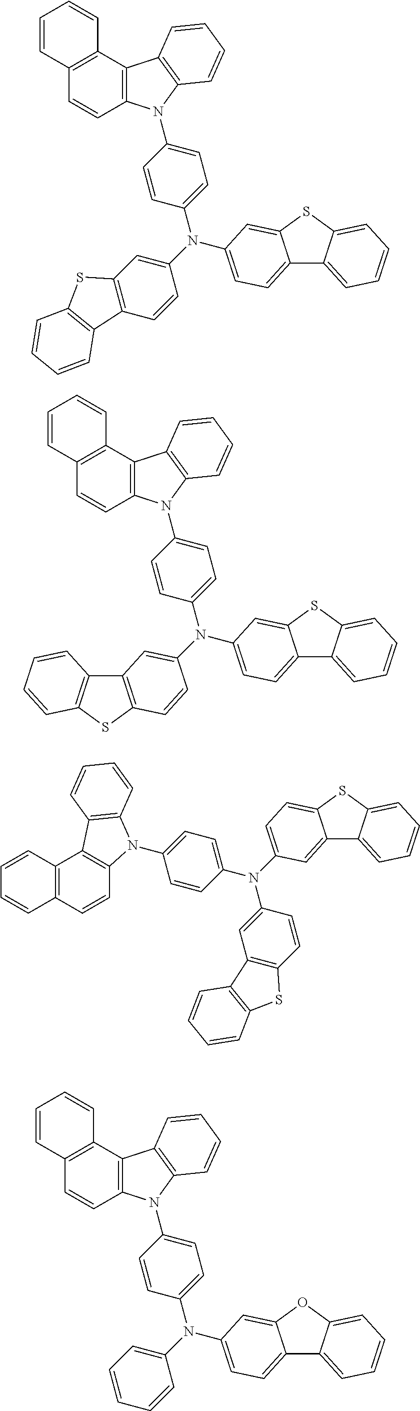

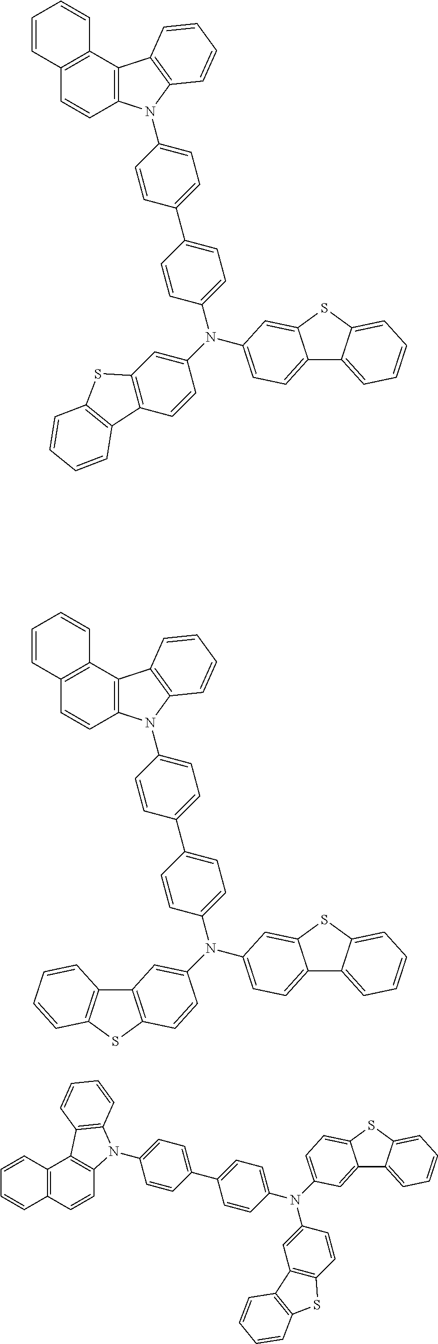

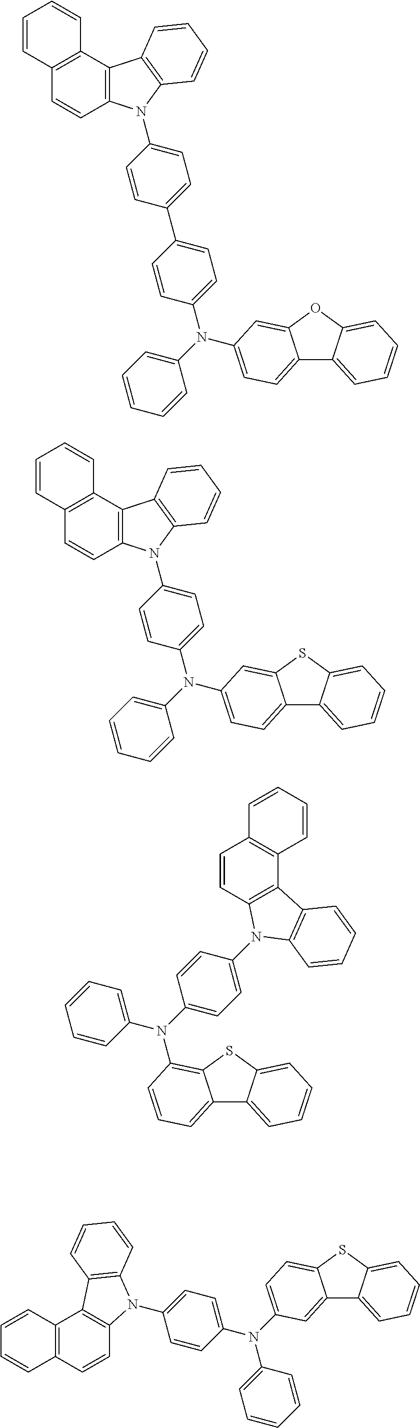 [SMILES: c1ccc(N(c2ccc(-c3ccc(-n4c5ccccc5c5c6ccccc6ccc54)cc3)cc2)c2ccc3c(c2)oc2ccccc23)cc1 ; c1ccc(N(c2ccc(-n3c4ccccc4c4c5ccccc5ccc43)cc2)c2ccc3c(c2)sc2ccccc23)cc1 ; c1ccc(N(c2ccc(-n3c4ccccc4c4c5ccccc5ccc43)cc2)c2cccc3c2sc2ccccc23)cc1 ; c1ccc(N(c2ccc(-n3c4ccccc4c4c5ccccc5ccc43)cc2)c2ccc3sc4ccccc4c3c2)cc1]

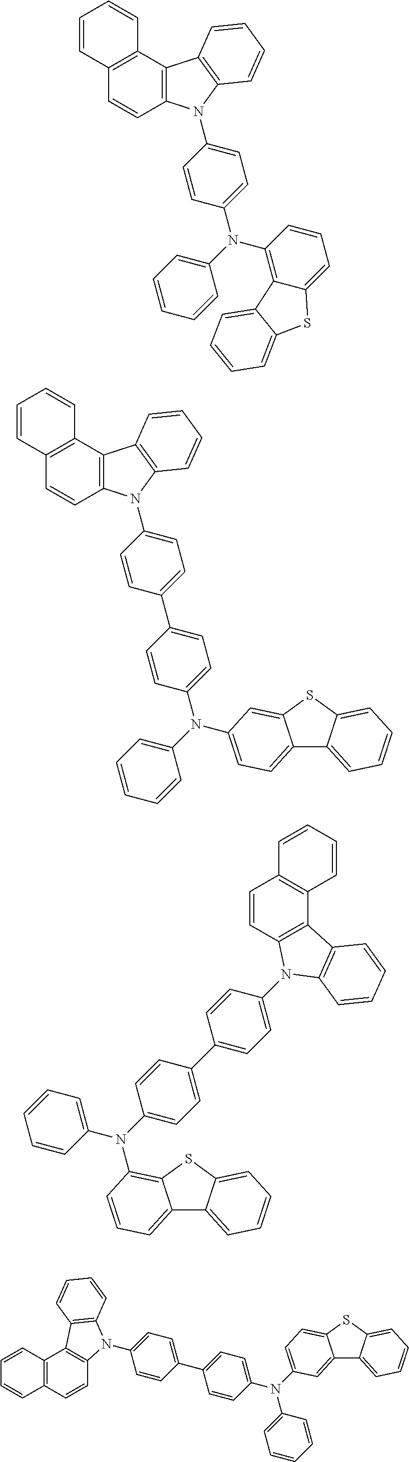 [SMILES: c1ccc(N(c2ccc(-n3c4ccccc4c4c5ccccc5ccc43)cc2)c2cccc3sc4ccccc4c23)cc1 ; c1ccc(N(c2ccc(-c3ccc(-n4c5ccccc5c5c6ccccc6ccc54)cc3)cc2)c2ccc3c(c2)sc2ccccc23)cc1 ; c1ccc(N(c2ccc(-c3ccc(-n4c5ccccc5c5c6ccccc6ccc54)cc3)cc2)c2cccc3c2sc2ccccc23)cc1 ; c1ccc(N(c2ccc(-c3ccc(-n4c5ccccc5c5c6ccccc6ccc54)cc3)cc2)c2ccc3sc4ccccc4c3c2)cc1]

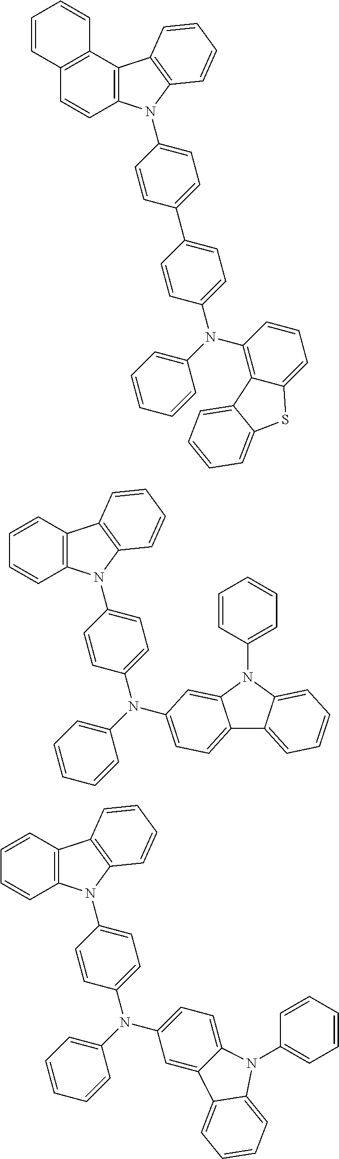

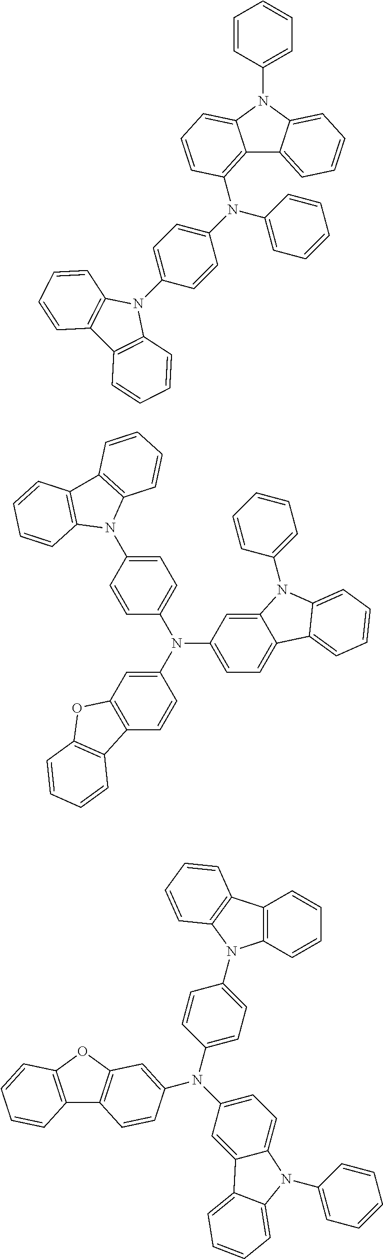

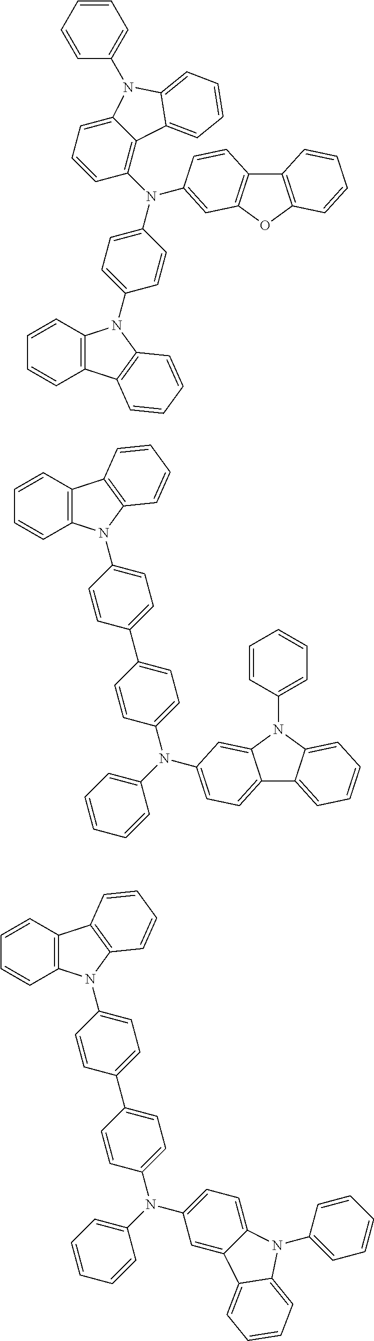

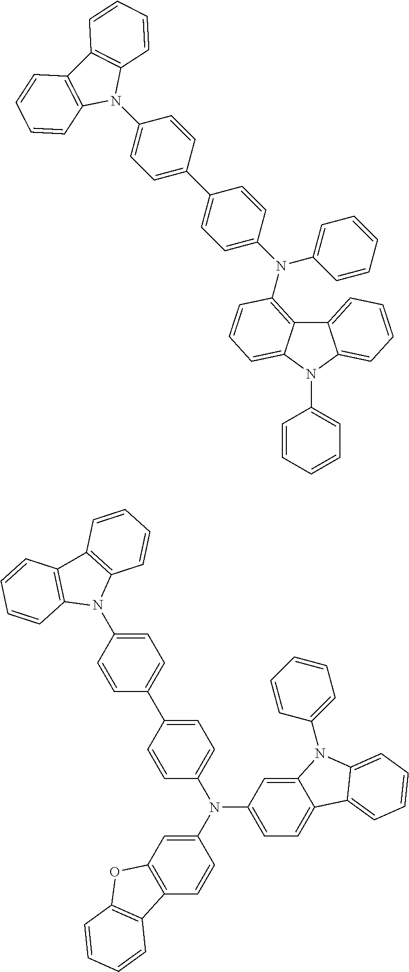 [SMILES: c1ccc(N(c2ccc(-c3ccc(-n4c5ccccc5c5ccccc54)cc3)cc2)c2cccc3c2c2ccccc2n3-c2ccccc2)cc1 ; c1ccc(-n2c3ccccc3c3ccc(N(c4ccc(-c5ccc(-n6c7ccccc7c7ccccc76)cc5)cc4)c4ccc5c(c4)oc4ccccc45)cc32)cc1]

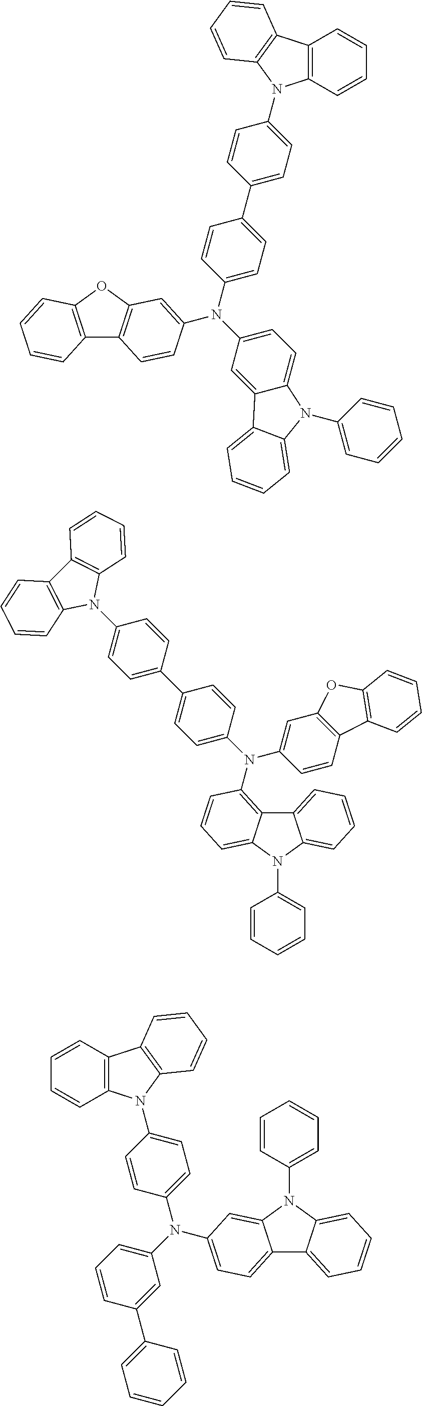 [SMILES: c1ccc(-n2c3ccccc3c3cc(N(c4ccc(-c5ccc(-n6c7ccccc7c7ccccc76)cc5)cc4)c4ccc5c(c4)oc4ccccc45)ccc32)cc1 ; c1ccc(-n2c3ccccc3c3c(N(c4ccc(-c5ccc(-n6c7ccccc7c7ccccc76)cc5)cc4)c4ccc5c(c4)oc4ccccc45)cccc32)cc1 ; c1ccc(-c2cccc(N(c3ccc(-n4c5ccccc5c5ccccc54)cc3)c3ccc4c5ccccc5n(-c5ccccc5)c4c3)c2)cc1]

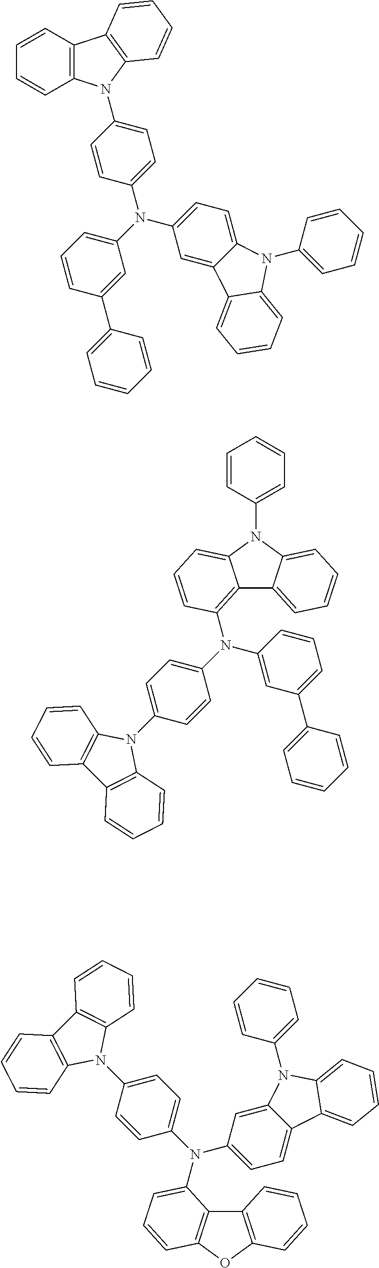 [SMILES: c1ccc(-c2cccc(N(c3ccc(-n4c5ccccc5c5ccccc54)cc3)c3ccc4c(c3)c3ccccc3n4-c3ccccc3)c2)cc1 ; c1ccc(-c2cccc(N(c3ccc(-n4c5ccccc5c5ccccc54)cc3)c3cccc4c3c3ccccc3n4-c3ccccc3)c2)cc1 ; c1ccc(-n2c3ccccc3c3ccc(N(c4ccc(-n5c6ccccc6c6ccccc65)cc4)c4cccc5oc6ccccc6c45)cc32)cc1]

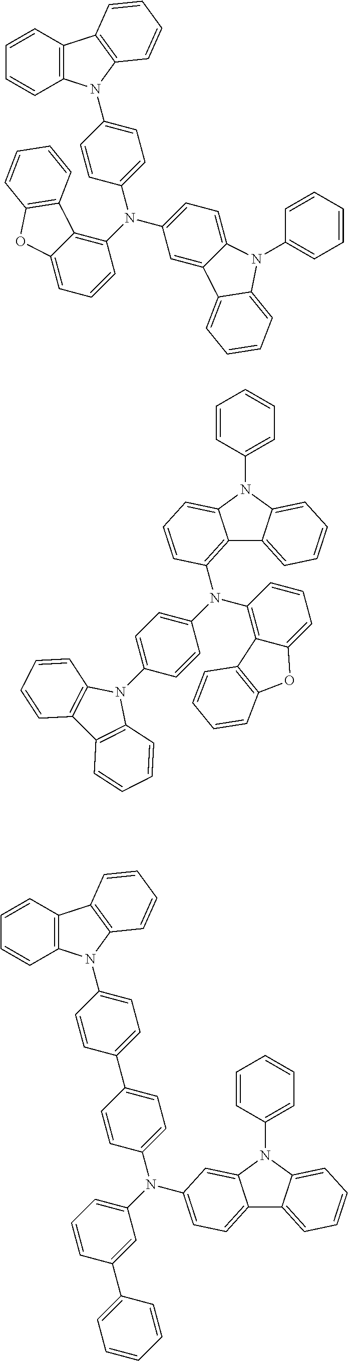

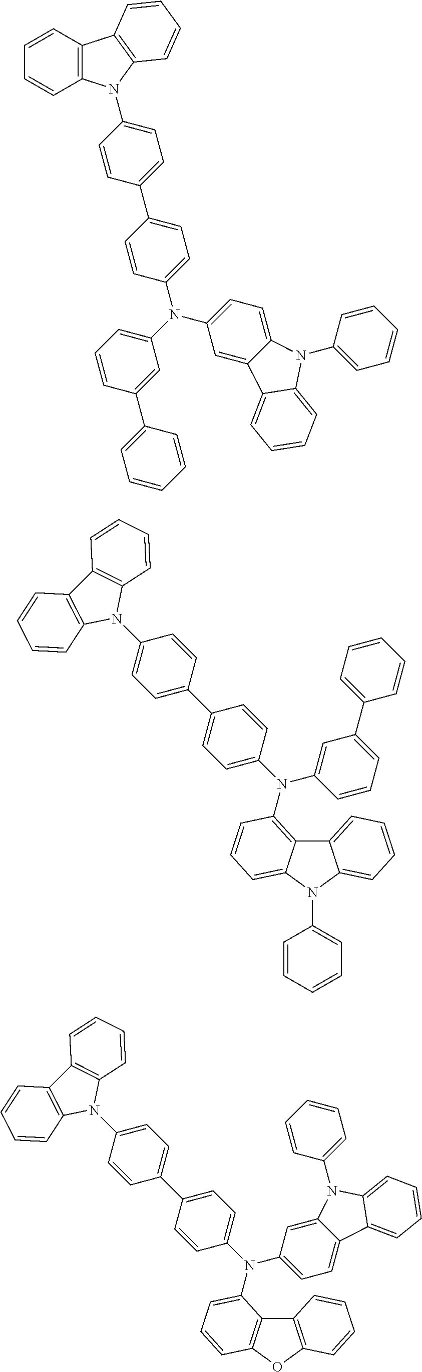

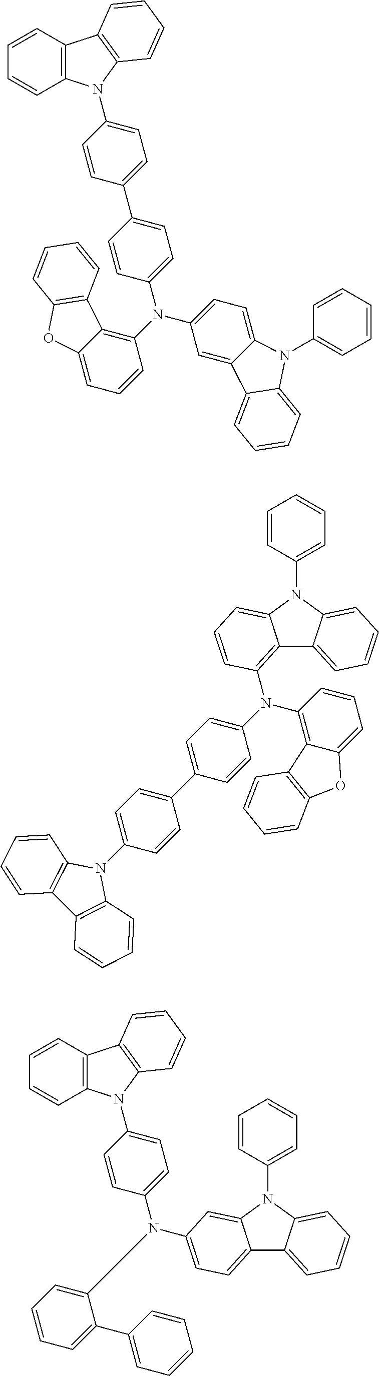 [SMILES: c1ccc(-n2c3ccccc3c3cc(N(c4ccc(-c5ccc(-n6c7ccccc7c7ccccc76)cc5)cc4)c4cccc5oc6ccccc6c45)ccc32)cc1 ; c1ccc(-n2c3ccccc3c3c(N(c4ccc(-c5ccc(-n6c7ccccc7c7ccccc76)cc5)cc4)c4cccc5oc6ccccc6c45)cccc32)cc1 ; c1ccc(-c2ccccc2N(c2ccc(-n3c4ccccc4c4ccccc43)cc2)c2ccc3c4ccccc4n(-c4ccccc4)c3c2)cc1]

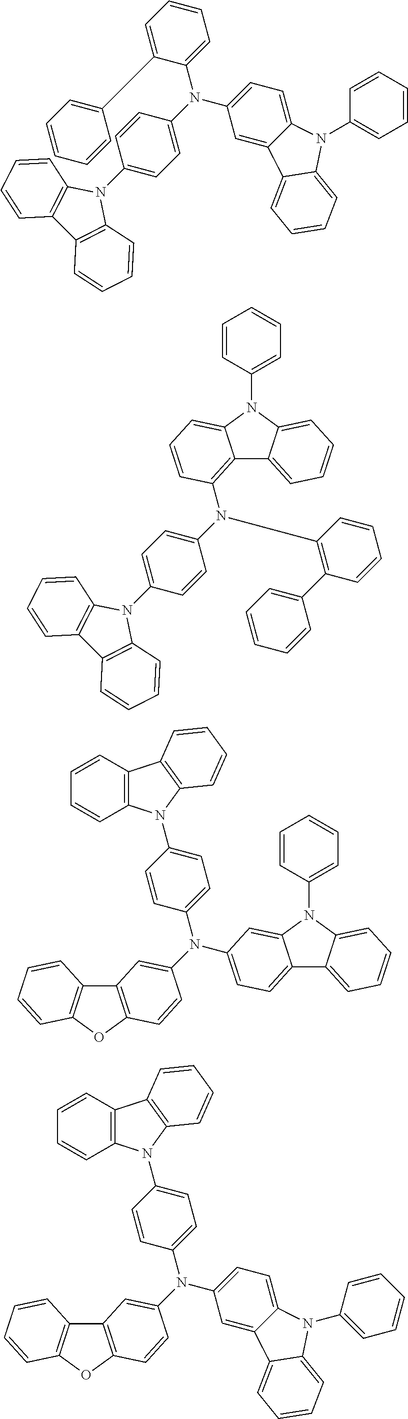

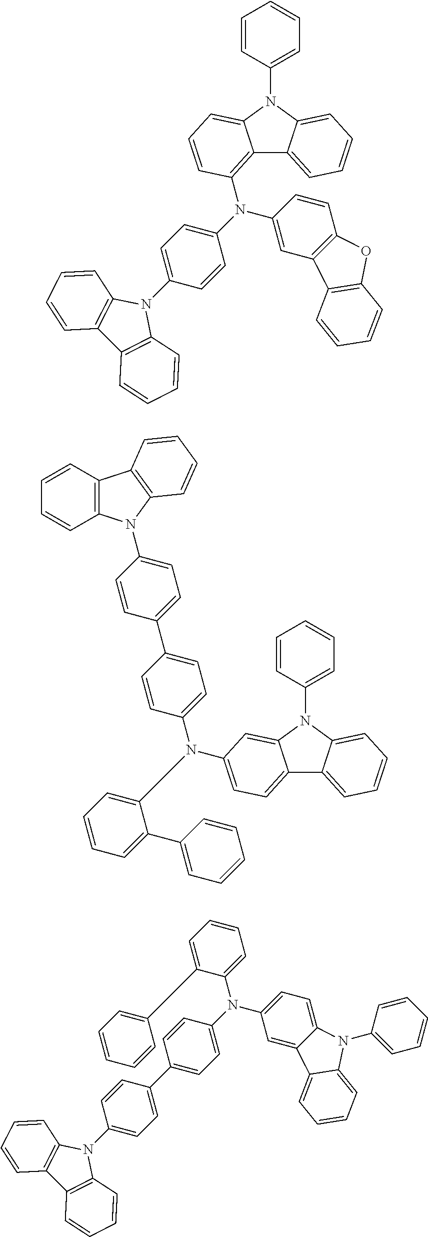

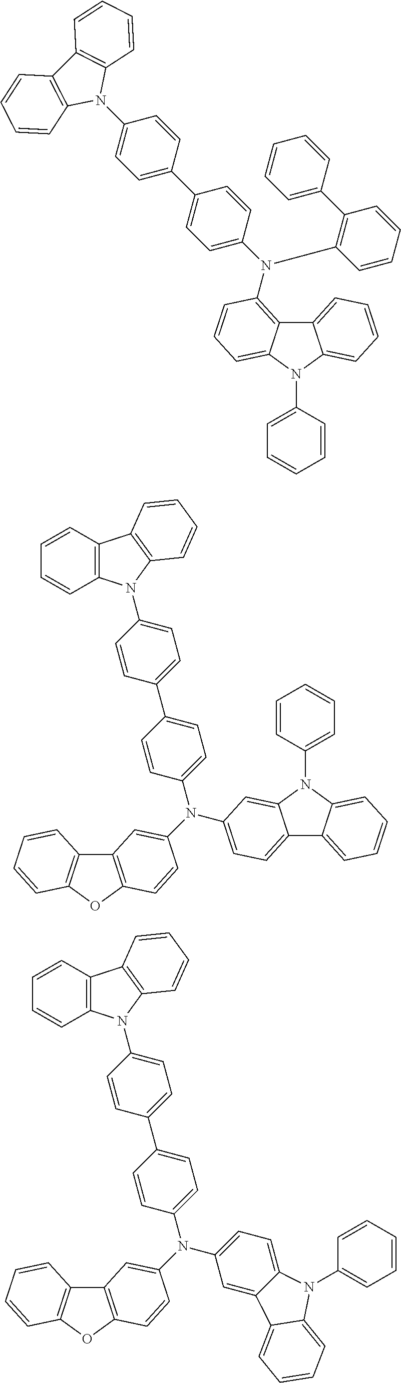

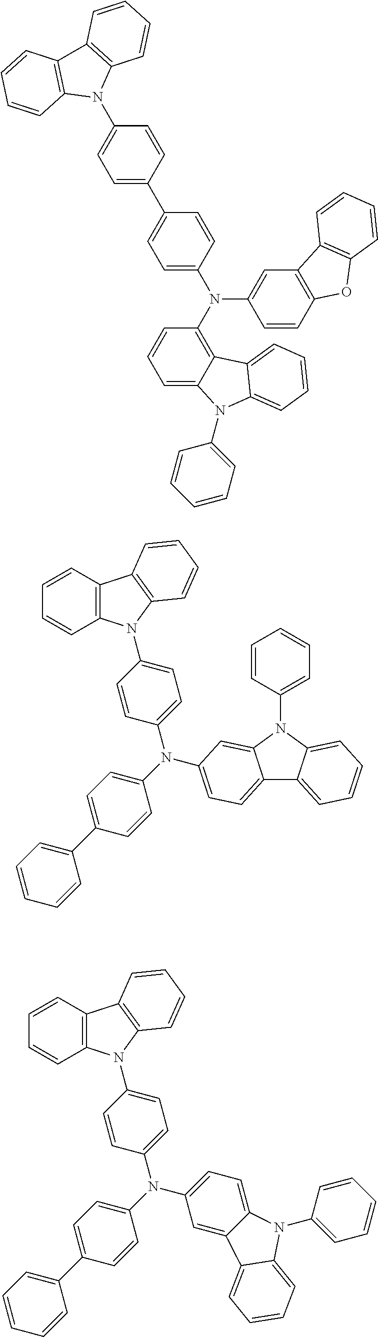

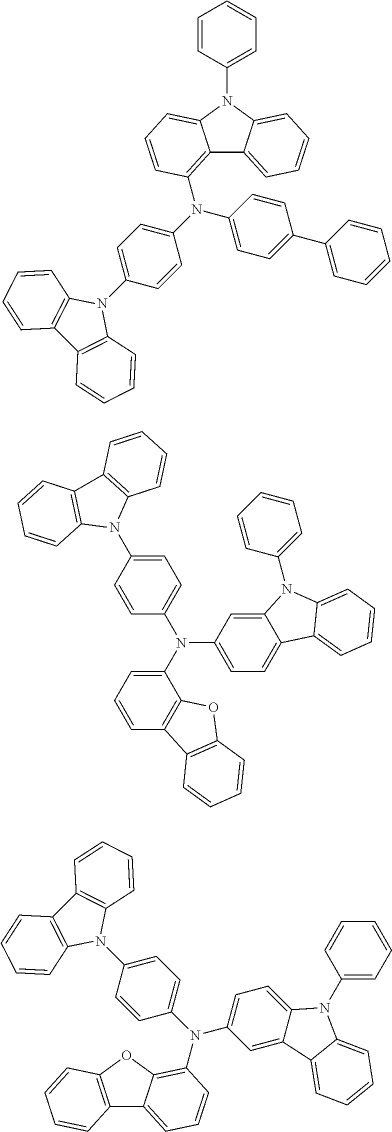

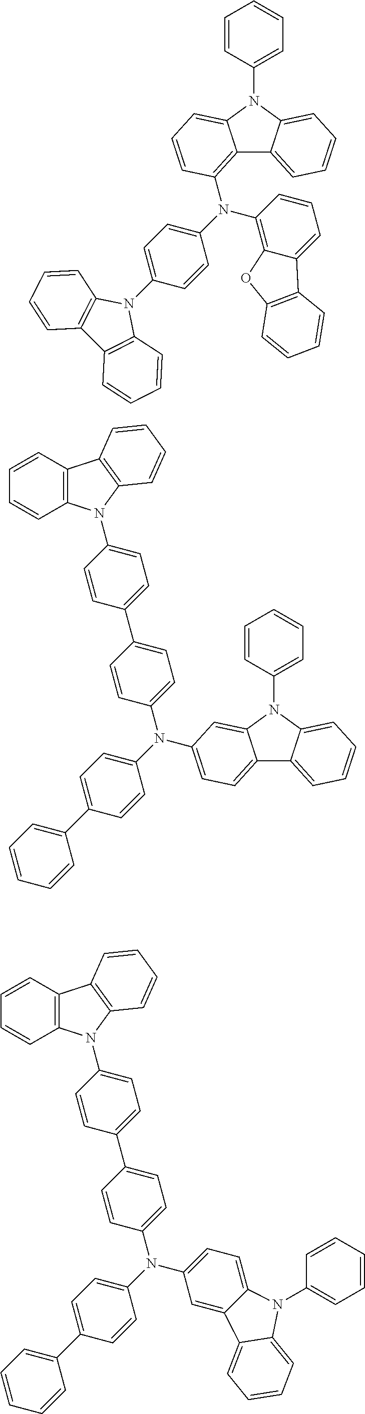

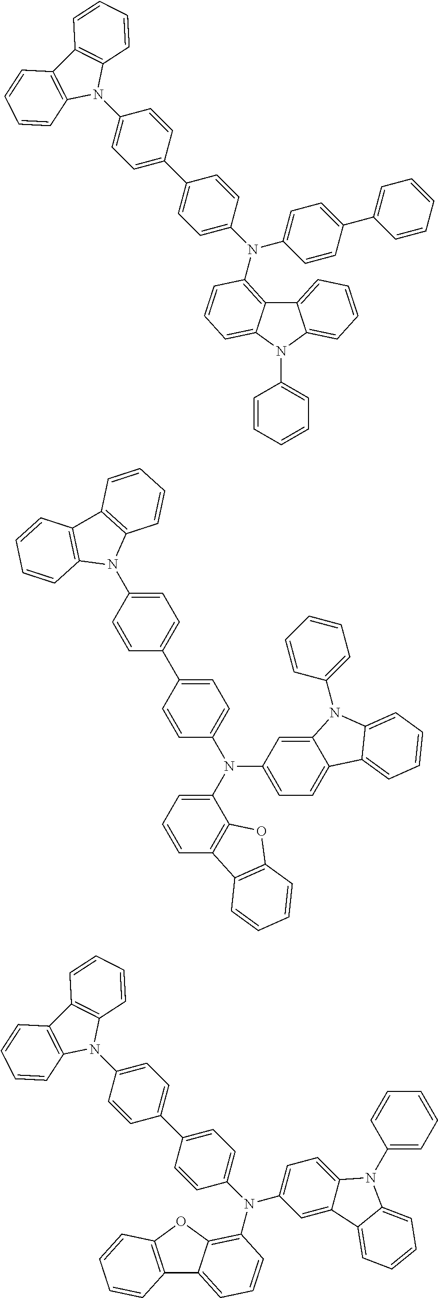 [SMILES: c1ccc(-c2ccc(N(c3ccc(-c4ccc(-n5c6ccccc6c6ccccc65)cc4)cc3)c3cccc4c3c3ccccc3n4-c3ccccc3)cc2)cc1 ; c1ccc(-n2c3ccccc3c3ccc(N(c4ccc(-c5ccc(-n6c7ccccc7c7ccccc76)cc5)cc4)c4cccc5c4oc4ccccc45)cc32)cc1 ; c1ccc(-n2c3ccccc3c3cc(N(c4ccc(-c5ccc(-n6c7ccccc7c7ccccc76)cc5)cc4)c4cccc5c4oc4ccccc45)ccc32)cc1]

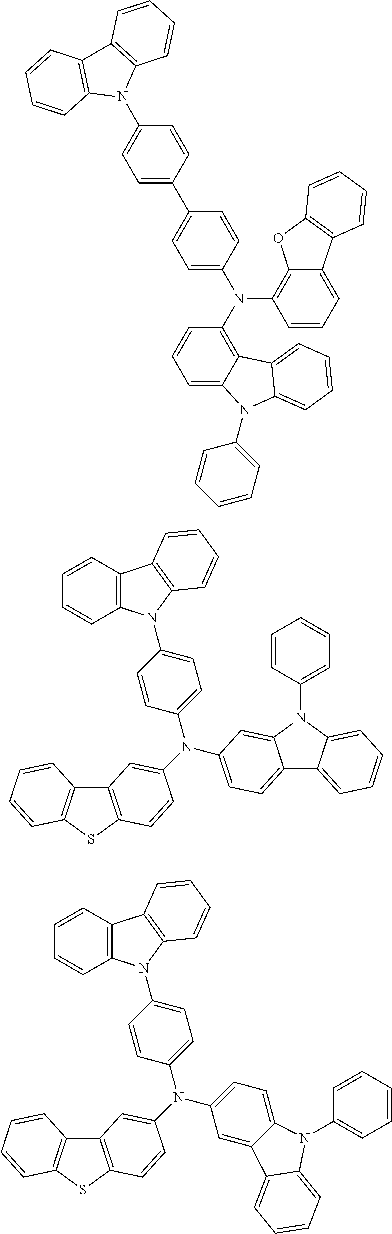

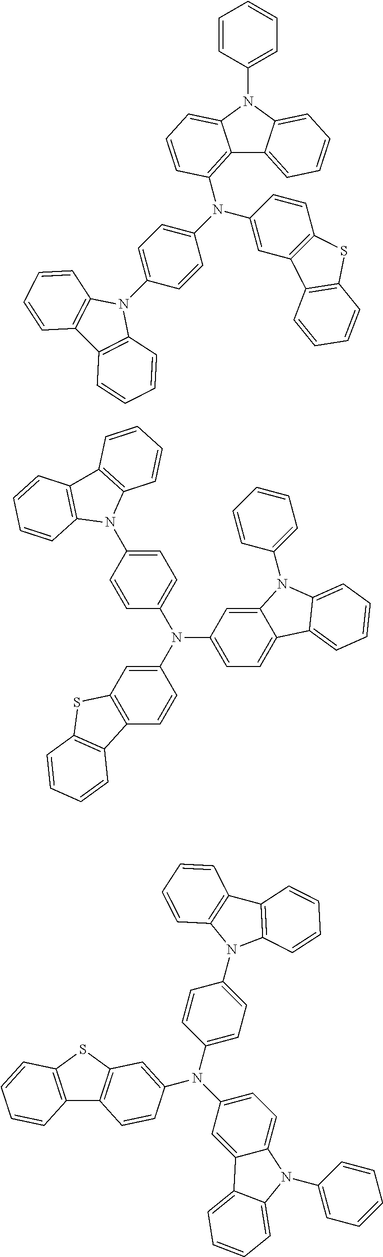

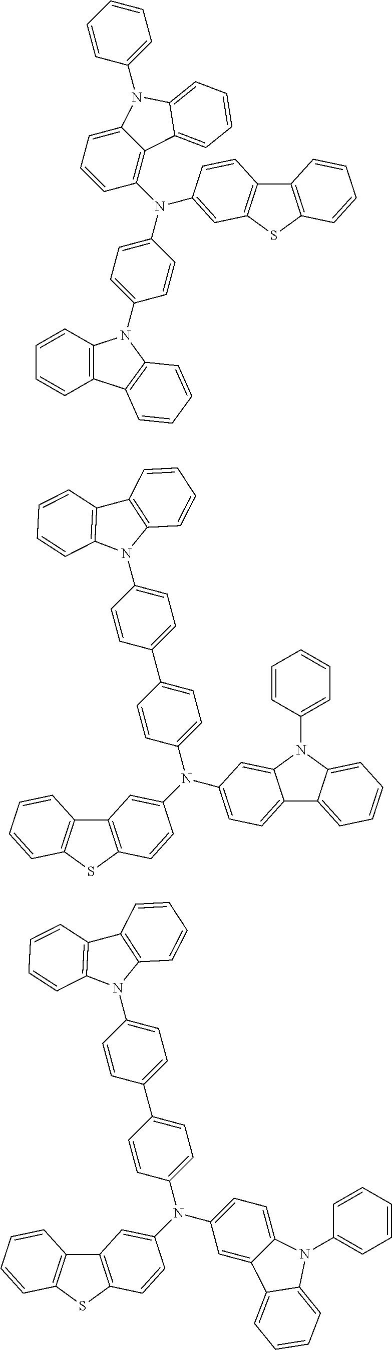

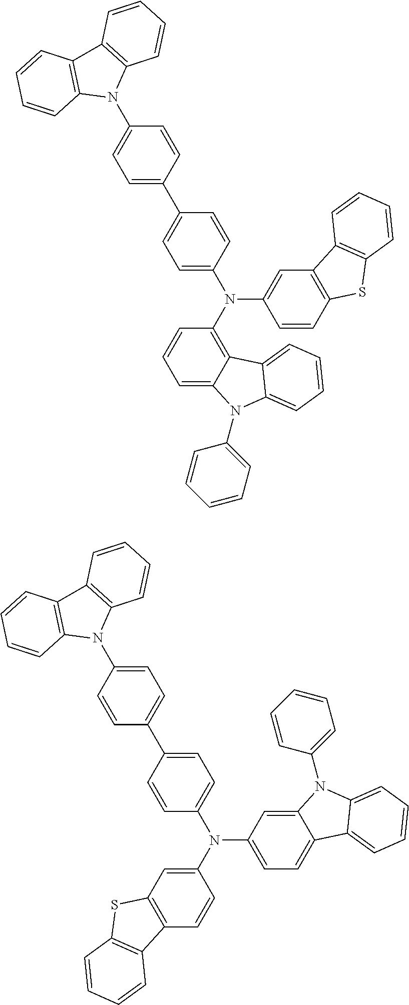 [SMILES: c1ccc(-n2c3ccccc3c3c(N(c4ccc(-c5ccc(-n6c7ccccc7c7ccccc76)cc5)cc4)c4ccc5sc6ccccc6c5c4)cccc32)cc1 ; c1ccc(-n2c3ccccc3c3ccc(N(c4ccc(-c5ccc(-n6c7ccccc7c7ccccc76)cc5)cc4)c4ccc5c(c4)sc4ccccc45)cc32)cc1]

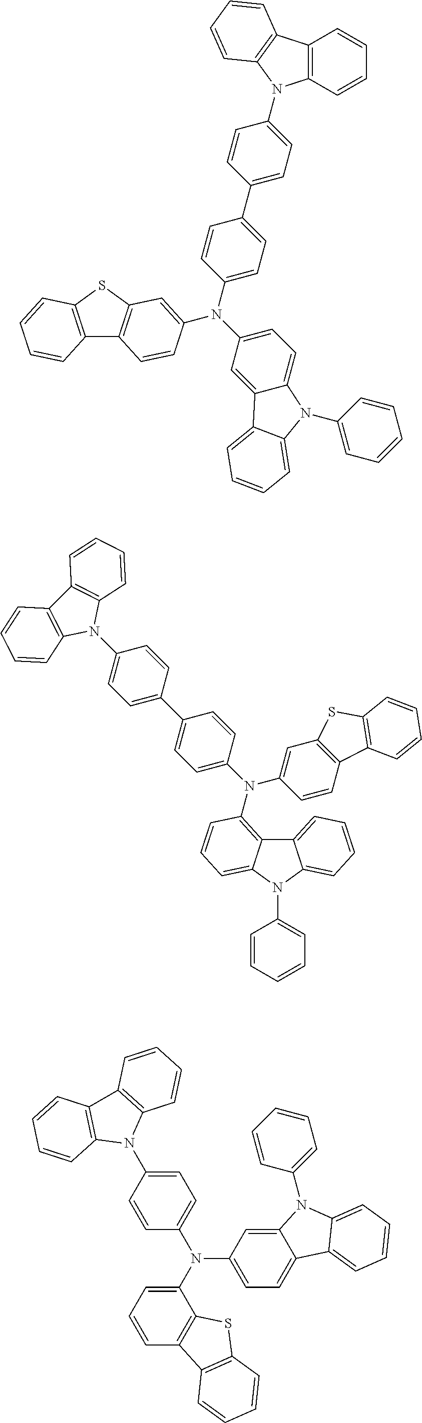 [SMILES: c1ccc(-n2c3ccccc3c3cc(N(c4ccc(-c5ccc(-n6c7ccccc7c7ccccc76)cc5)cc4)c4ccc5c(c4)sc4ccccc45)ccc32)cc1 ; c1ccc(-n2c3ccccc3c3c(N(c4ccc(-c5ccc(-n6c7ccccc7c7ccccc76)cc5)cc4)c4ccc5c(c4)sc4ccccc45)cccc32)cc1 ; c1ccc(-n2c3ccccc3c3ccc(N(c4ccc(-n5c6ccccc6c6ccccc65)cc4)c4cccc5c4sc4ccccc45)cc32)cc1]

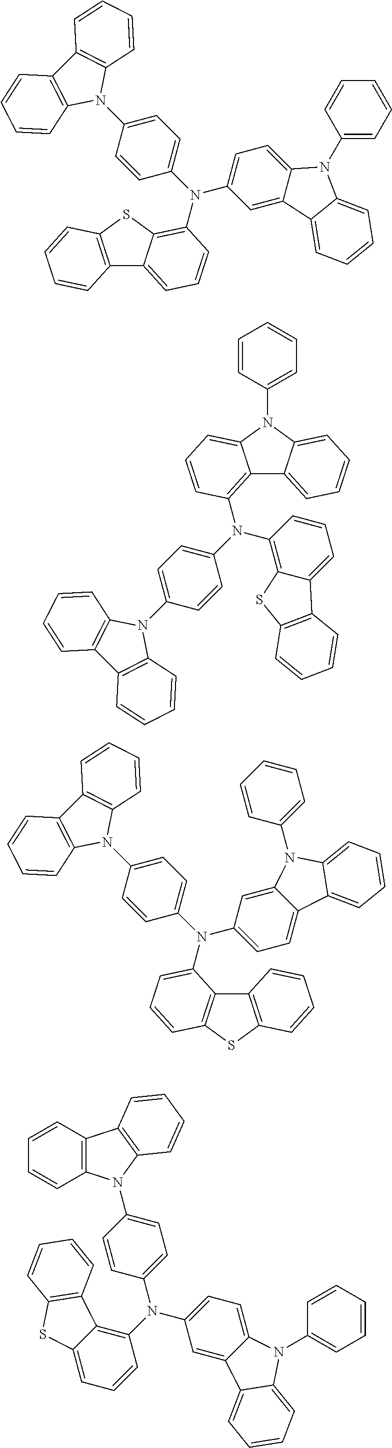

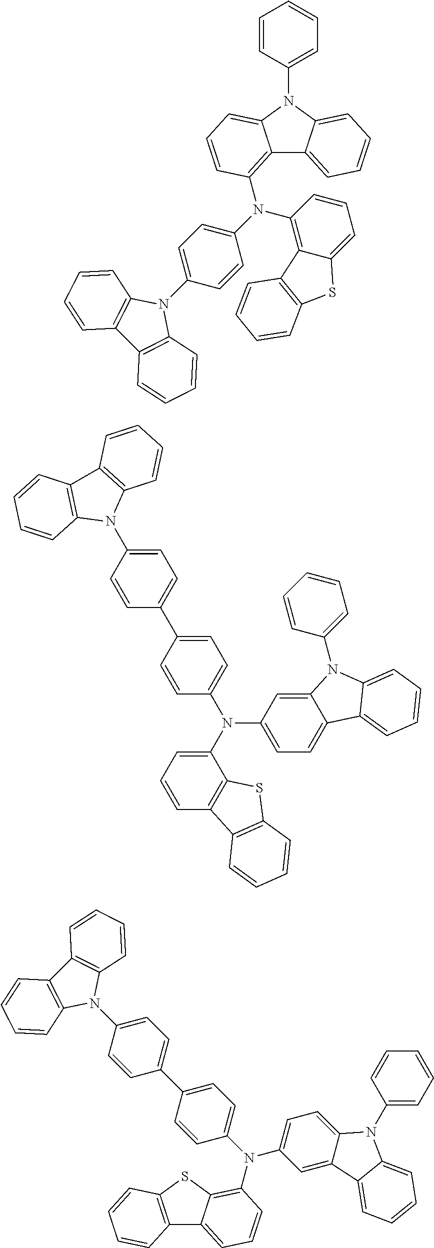

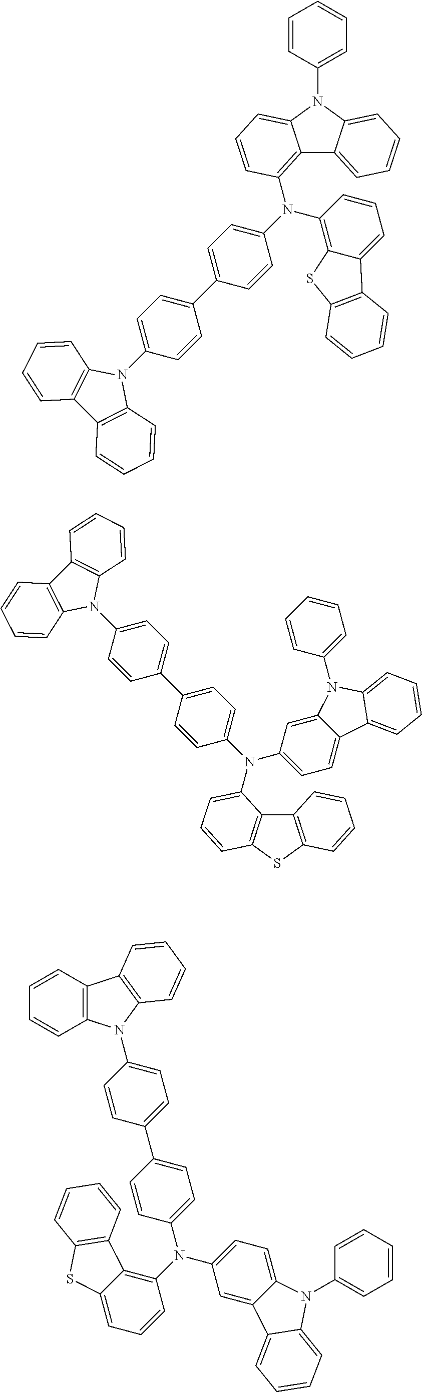 [SMILES: c1ccc(-n2c3ccccc3c3c(N(c4ccc(-c5ccc(-n6c7ccccc7c7ccccc76)cc5)cc4)c4cccc5c4sc4ccccc45)cccc32)cc1 ; c1ccc(-n2c3ccccc3c3ccc(N(c4ccc(-c5ccc(-n6c7ccccc7c7ccccc76)cc5)cc4)c4cccc5sc6ccccc6c45)cc32)cc1 ; c1ccc(-n2c3ccccc3c3cc(N(c4ccc(-c5ccc(-n6c7ccccc7c7ccccc76)cc5)cc4)c4cccc5sc6ccccc6c45)ccc32)cc1]

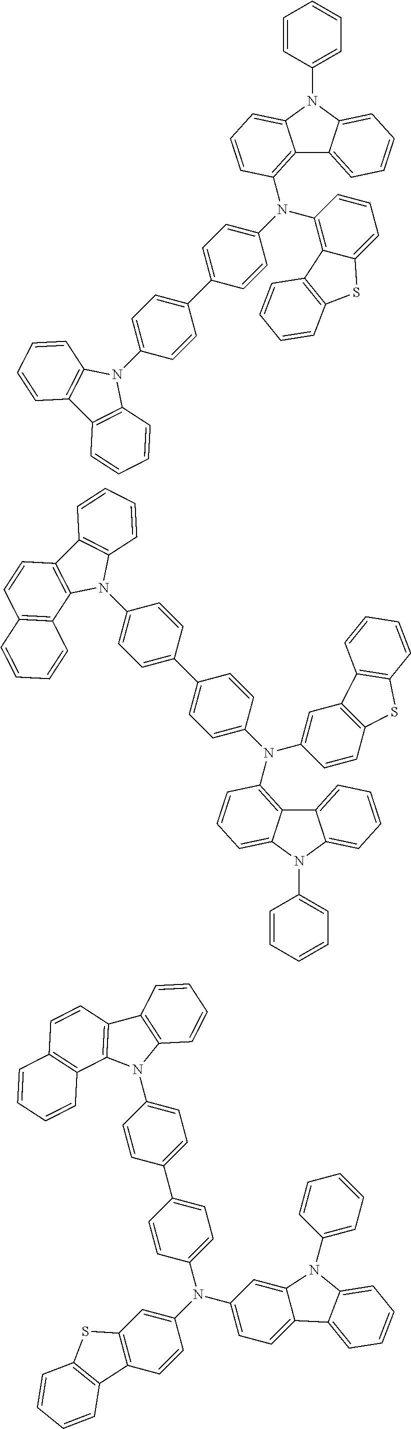

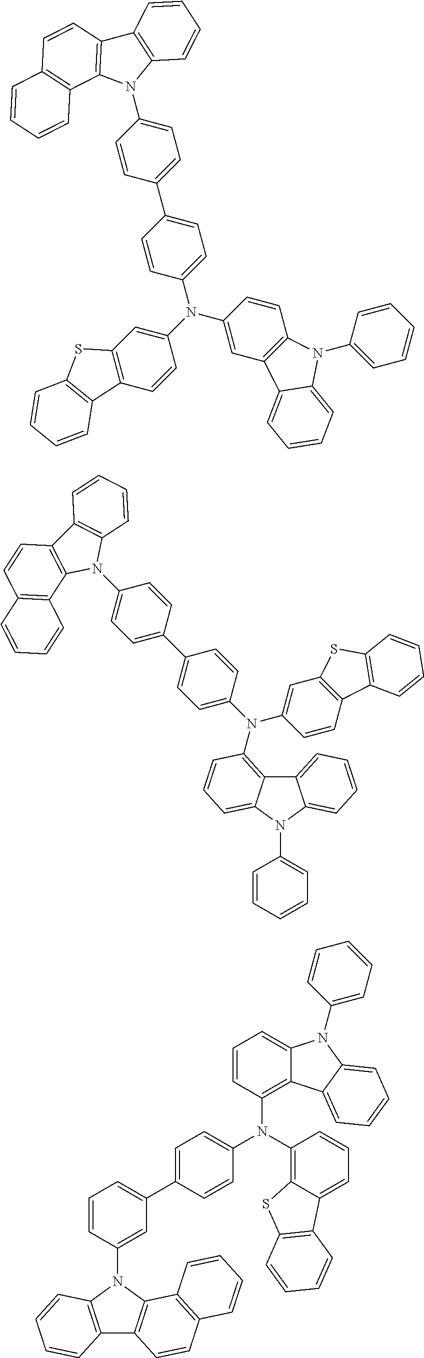

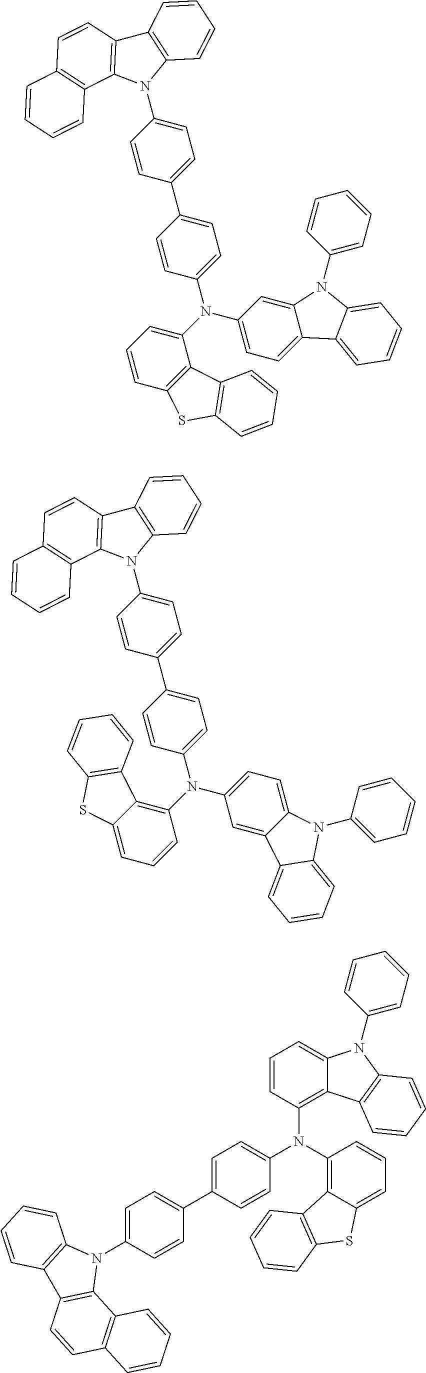 [SMILES: c1ccc(-n2c3ccccc3c3ccc(N(c4ccc(-c5ccc(-n6c7ccccc7c7ccc8ccccc8c76)cc5)cc4)c4cccc5sc6ccccc6c45)cc32)cc1 ; c1ccc(-n2c3ccccc3c3cc(N(c4ccc(-c5ccc(-n6c7ccccc7c7ccc8ccccc8c76)cc5)cc4)c4cccc5sc6ccccc6c45)ccc32)cc1 ; c1ccc(-n2c3ccccc3c3c(N(c4ccc(-c5ccc(-n6c7ccccc7c7ccc8ccccc8c76)cc5)cc4)c4cccc5sc6ccccc6c45)cccc32)cc1]

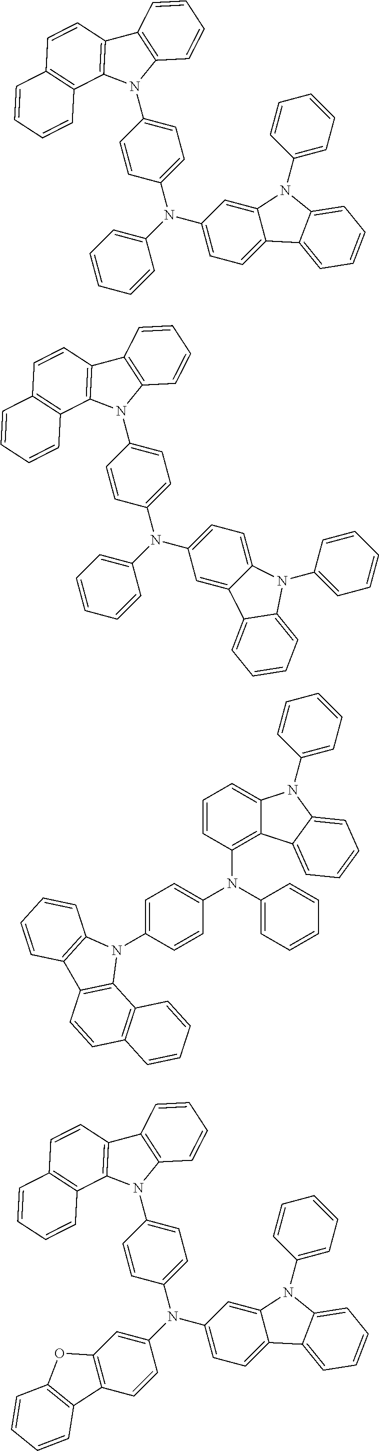

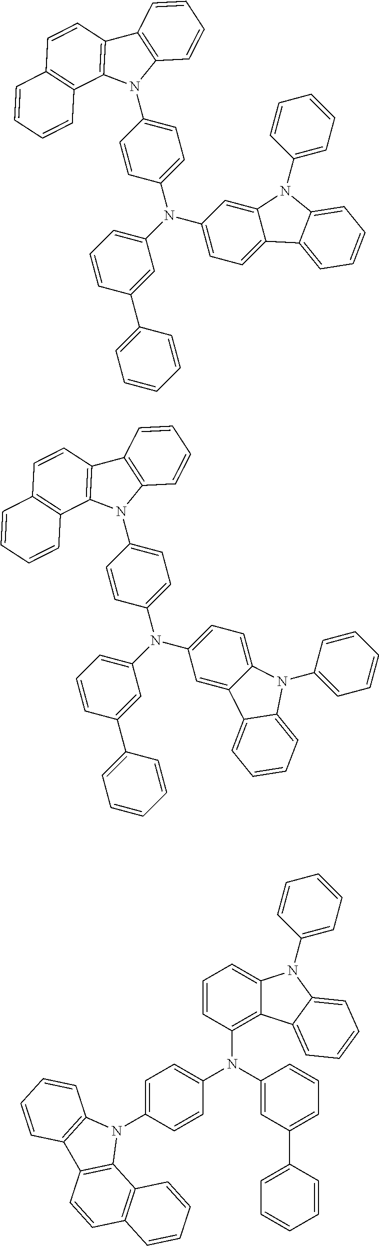

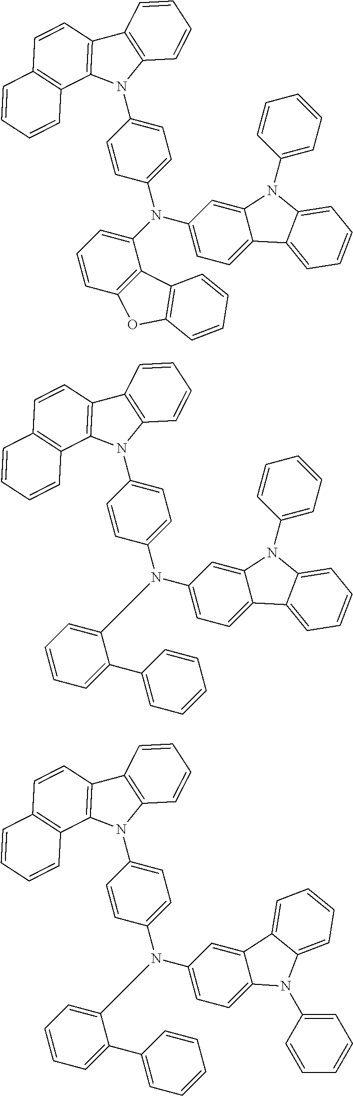

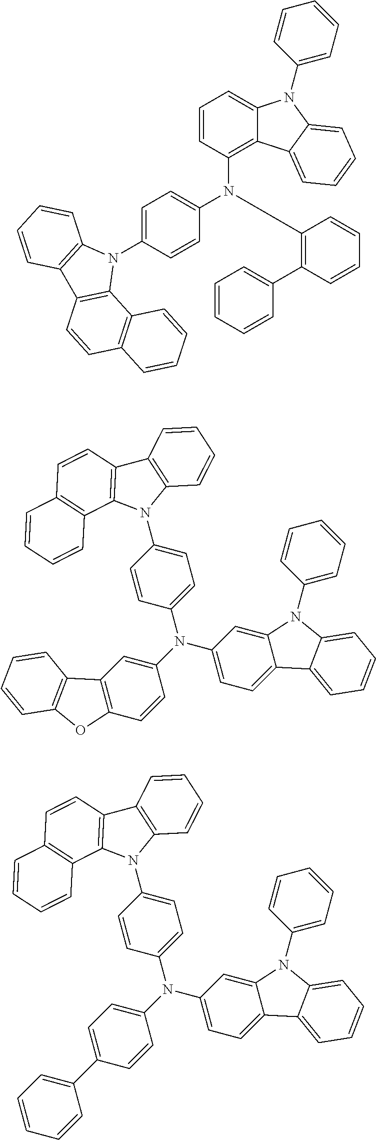 [SMILES: c1ccc(-c2ccccc2N(c2ccc(-n3c4ccccc4c4ccc5ccccc5c43)cc2)c2cccc3c2c2ccccc2n3-c2ccccc2)cc1 ; c1ccc(-n2c3ccccc3c3ccc(N(c4ccc(-n5c6ccccc6c6ccc7ccccc7c65)cc4)c4ccc5oc6ccccc6c5c4)cc32)cc1 ; c1ccc(-c2ccc(N(c3ccc(-n4c5ccccc5c5ccc6ccccc6c54)cc3)c3ccc4c5ccccc5n(-c5ccccc5)c4c3)cc2)cc1]

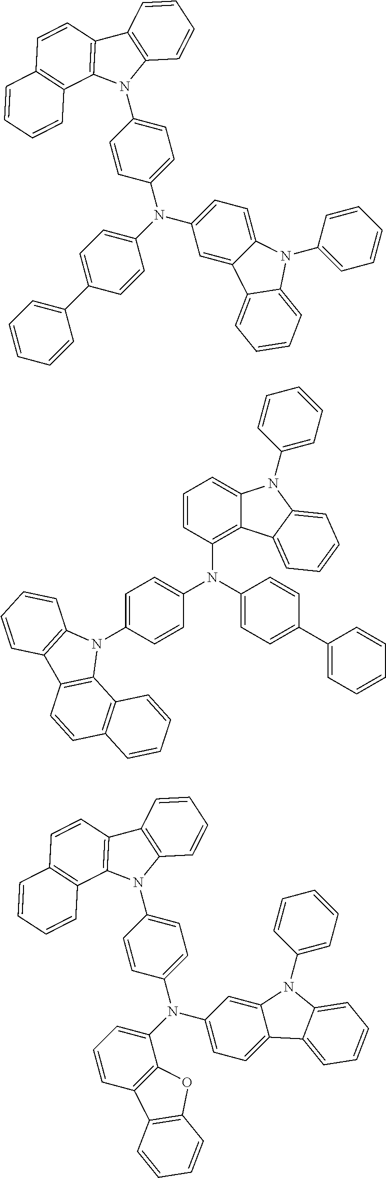

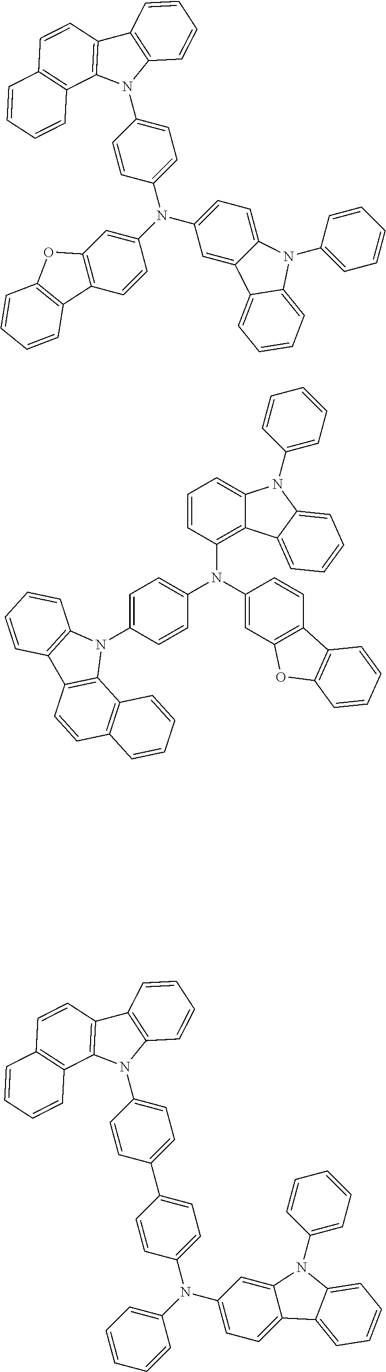

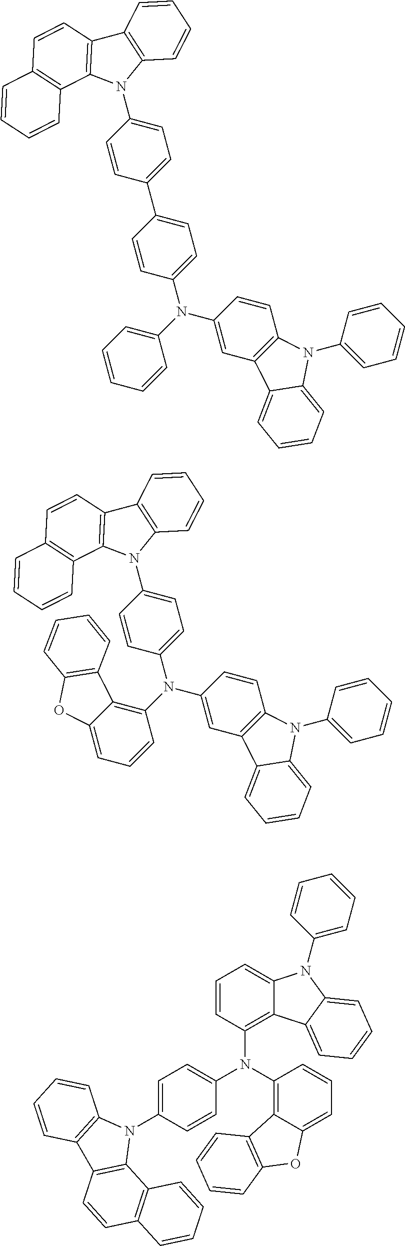 [SMILES: c1ccc(N(c2ccc(-c3ccc(-n4c5ccccc5c5ccc6ccccc6c54)cc3)cc2)c2ccc3c(c2)c2ccccc2n3-c2ccccc2)cc1 ; c1ccc(-n2c3ccccc3c3cc(N(c4ccc(-n5c6ccccc6c6ccc7ccccc7c65)cc4)c4cccc5oc6ccccc6c45)ccc32)cc1 ; c1ccc(-n2c3ccccc3c3c(N(c4ccc(-n5c6ccccc6c6ccc7ccccc7c65)cc4)c4cccc5oc6ccccc6c45)cccc32)cc1]

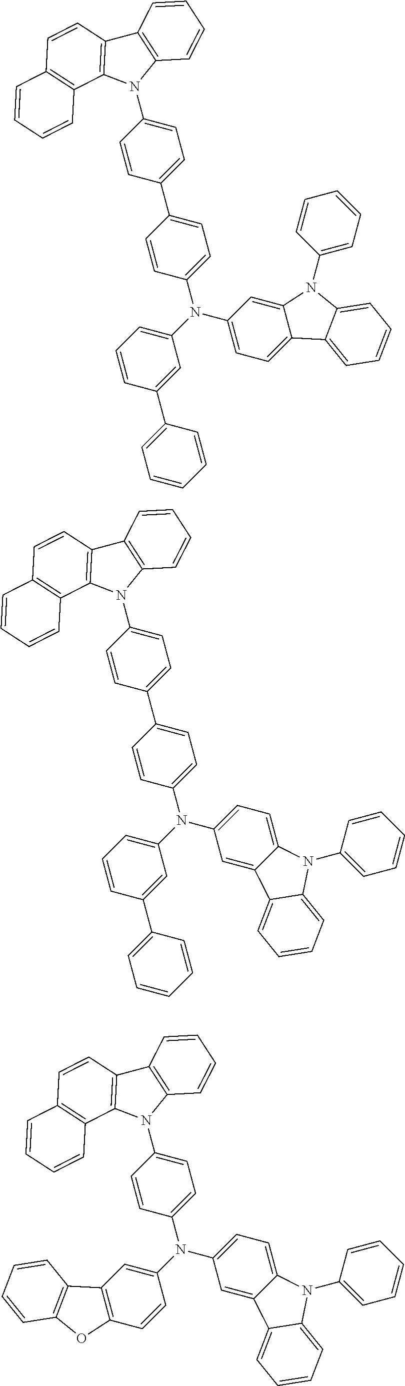

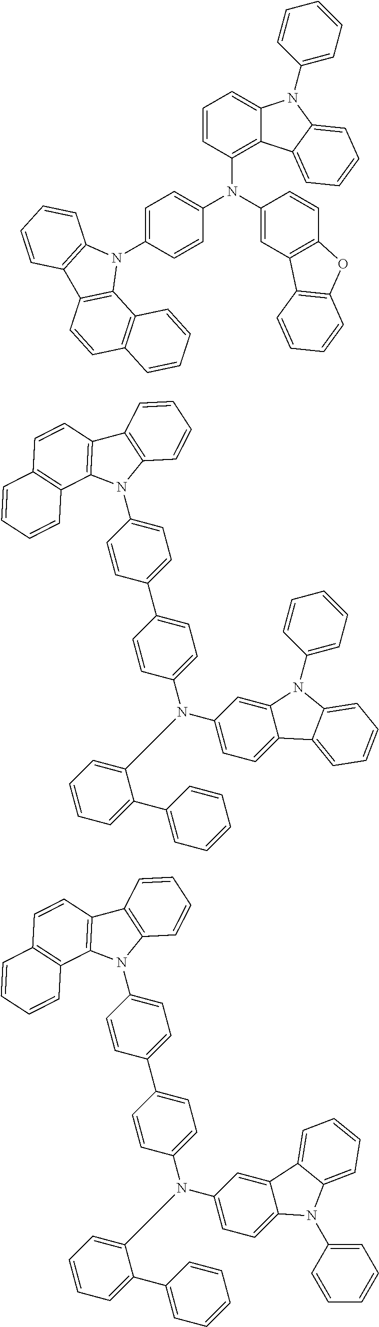

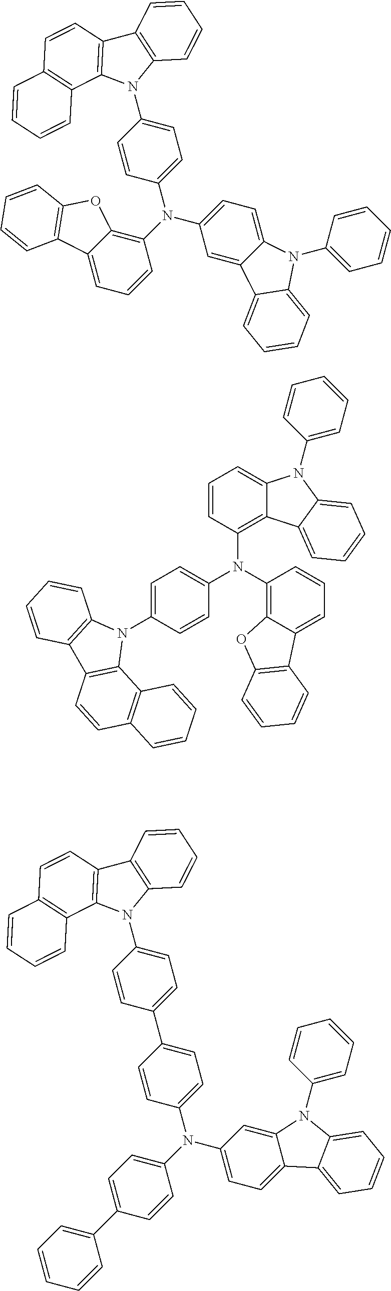

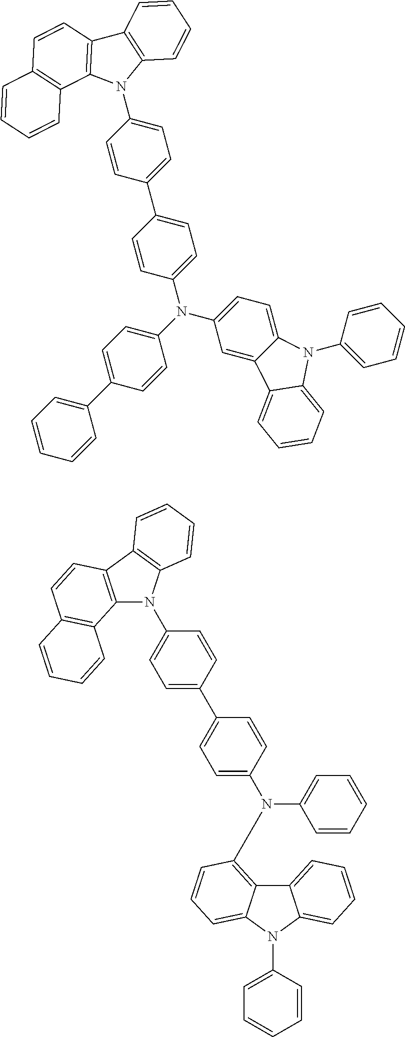 [SMILES: c1ccc(-c2ccc(N(c3ccc(-c4ccc(-n5c6ccccc6c6ccc7ccccc7c65)cc4)cc3)c3ccc4c(c3)c3ccccc3n4-c3ccccc3)cc2)cc1 ; c1ccc(N(c2ccc(-c3ccc(-n4c5ccccc5c5ccc6ccccc6c54)cc3)cc2)c2cccc3c2c2ccccc2n3-c2ccccc2)cc1]

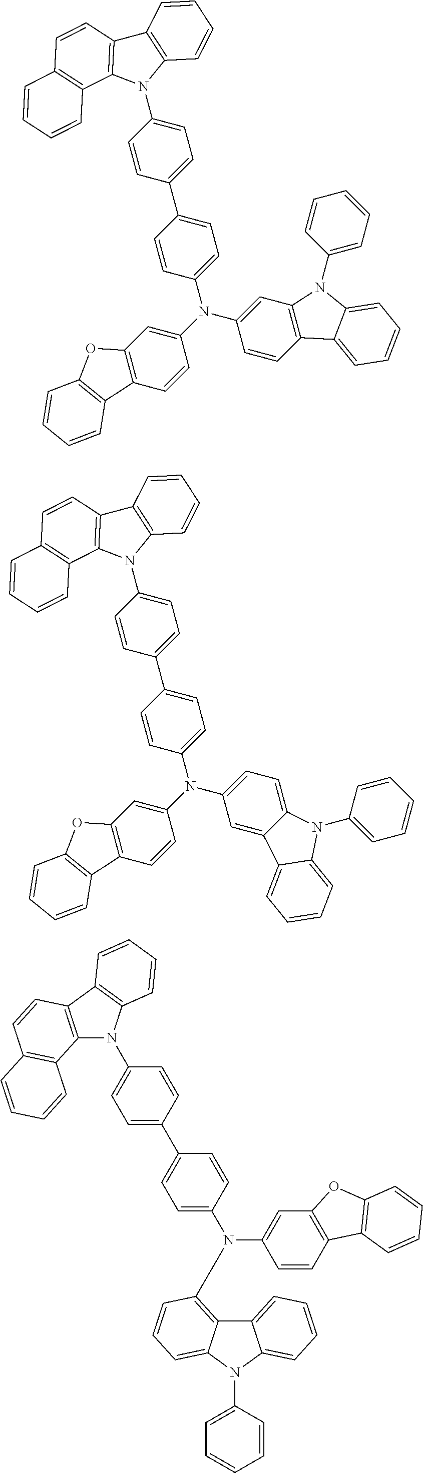 [SMILES: c1ccc(-n2c3ccccc3c3ccc(N(c4ccc(-c5ccc(-n6c7ccccc7c7ccc8ccccc8c76)cc5)cc4)c4ccc5c(c4)oc4ccccc45)cc32)cc1 ; c1ccc(-n2c3ccccc3c3cc(N(c4ccc(-c5ccc(-n6c7ccccc7c7ccc8ccccc8c76)cc5)cc4)c4ccc5c(c4)oc4ccccc45)ccc32)cc1 ; c1ccc(-n2c3ccccc3c3c(N(c4ccc(-c5ccc(-n6c7ccccc7c7ccc8ccccc8c76)cc5)cc4)c4ccc5c(c4)oc4ccccc45)cccc32)cc1]

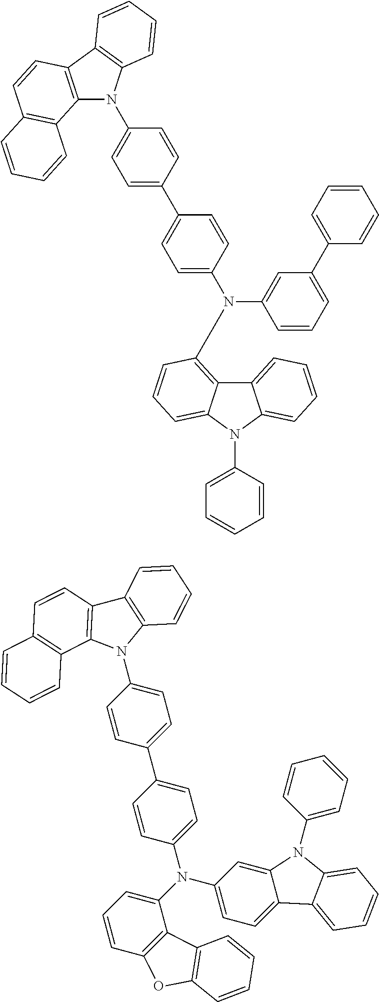 [SMILES: c1ccc(-c2cccc(N(c3ccc(-c4ccc(-n5c6ccccc6c6ccc7ccccc7c65)cc4)cc3)c3cccc4c3c3ccccc3n4-c3ccccc3)c2)cc1 ; c1ccc(-n2c3ccccc3c3ccc(N(c4ccc(-c5ccc(-n6c7ccccc7c7ccc8ccccc8c76)cc5)cc4)c4cccc5oc6ccccc6c45)cc32)cc1]

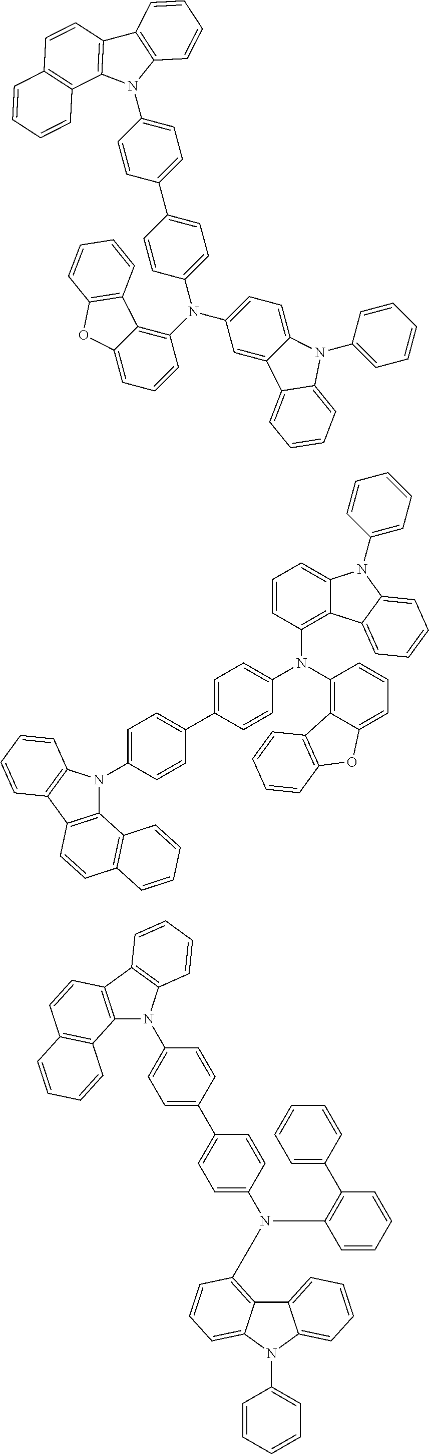

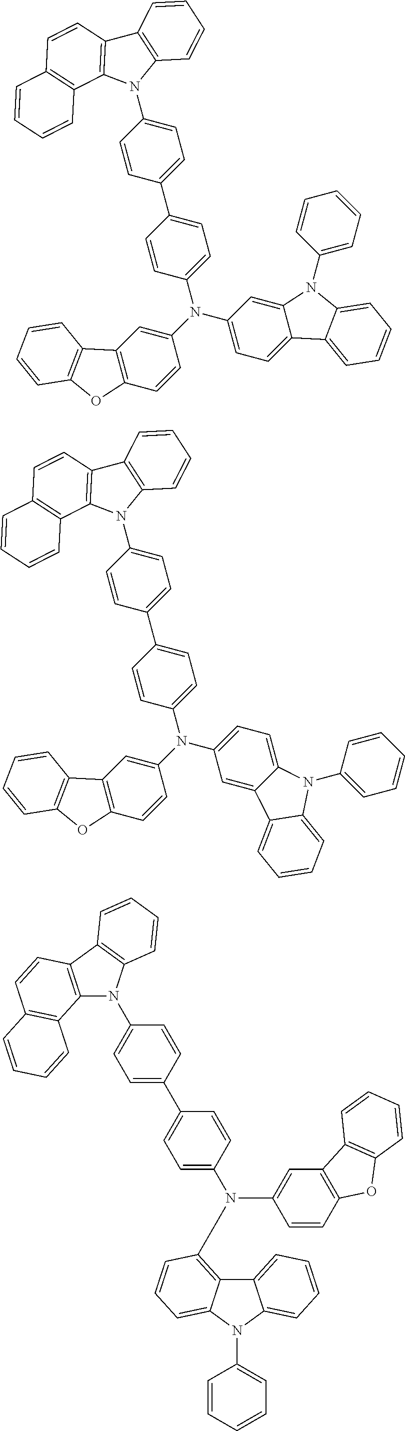

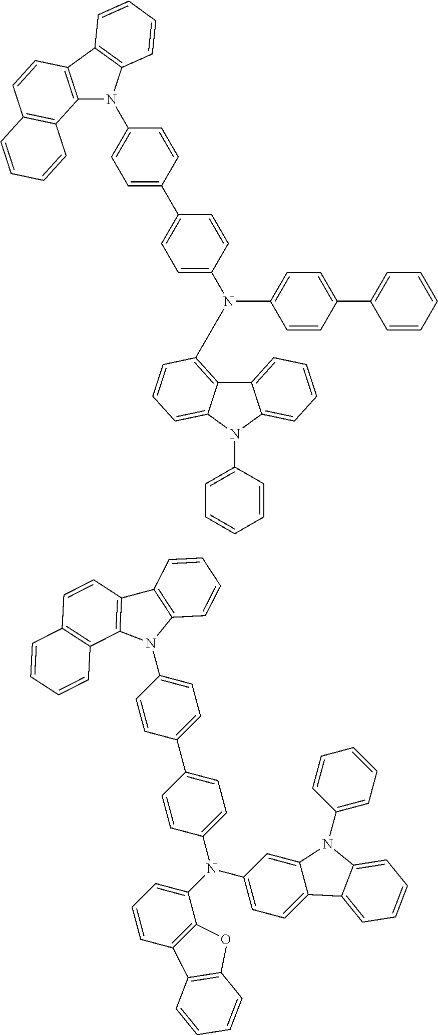

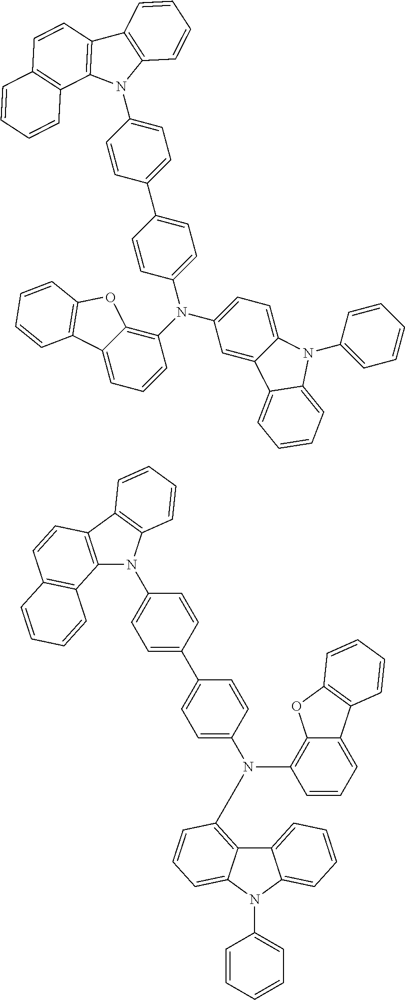 [SMILES: c1ccc(-n2c3ccccc3c3cc(N(c4ccc(-c5ccc(-n6c7ccccc7c7ccc8ccccc8c76)cc5)cc4)c4cccc5c4oc4ccccc45)ccc32)cc1 ; c1ccc(-n2c3ccccc3c3c(N(c4ccc(-c5ccc(-n6c7ccccc7c7ccc8ccccc8c76)cc5)cc4)c4cccc5c4oc4ccccc45)cccc32)cc1]

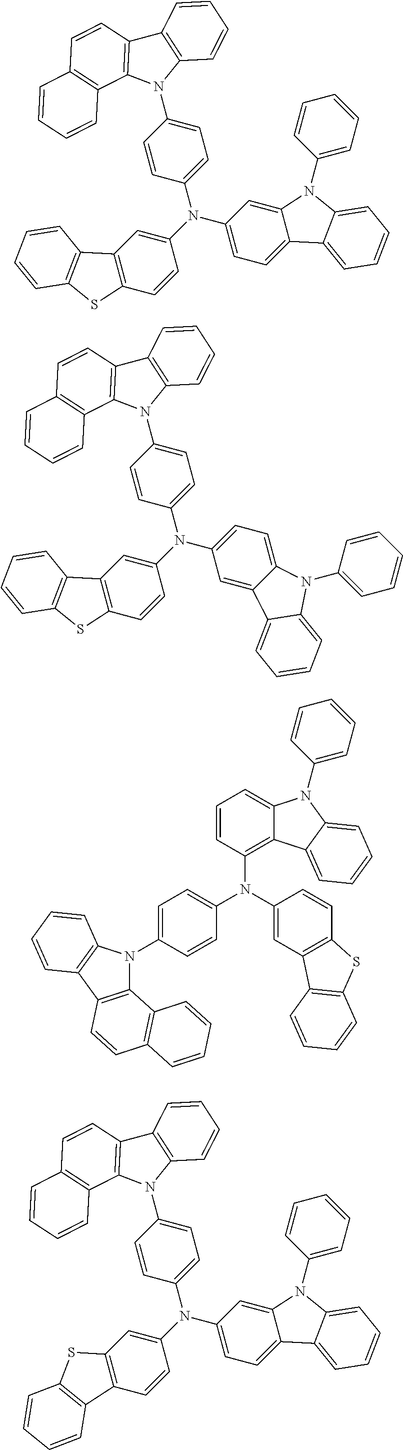

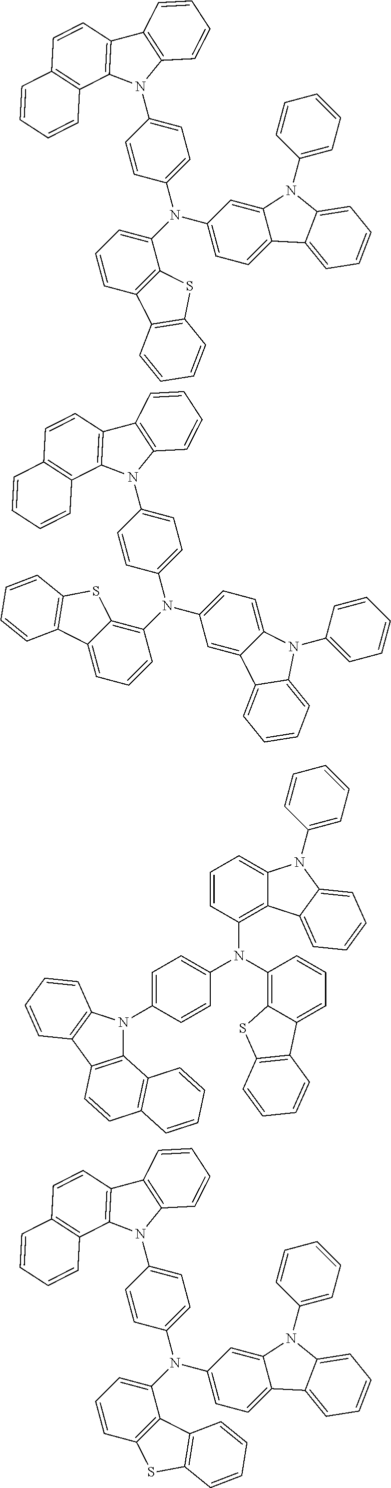

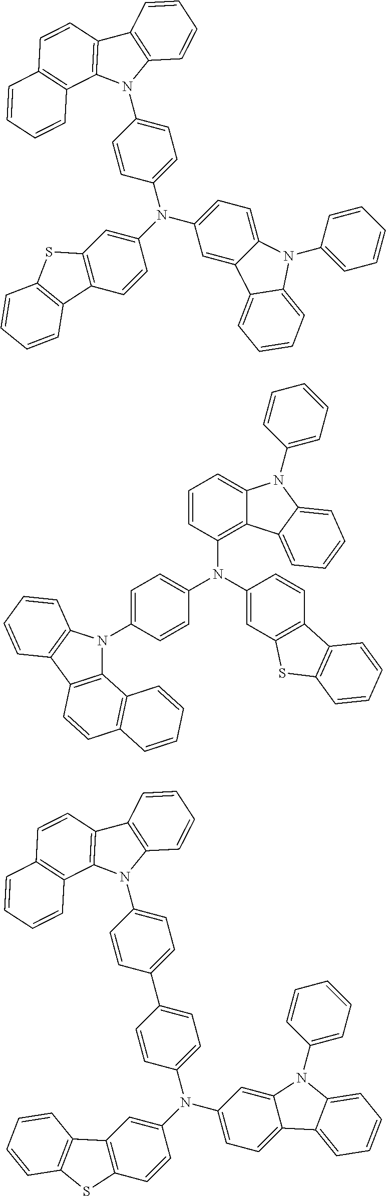

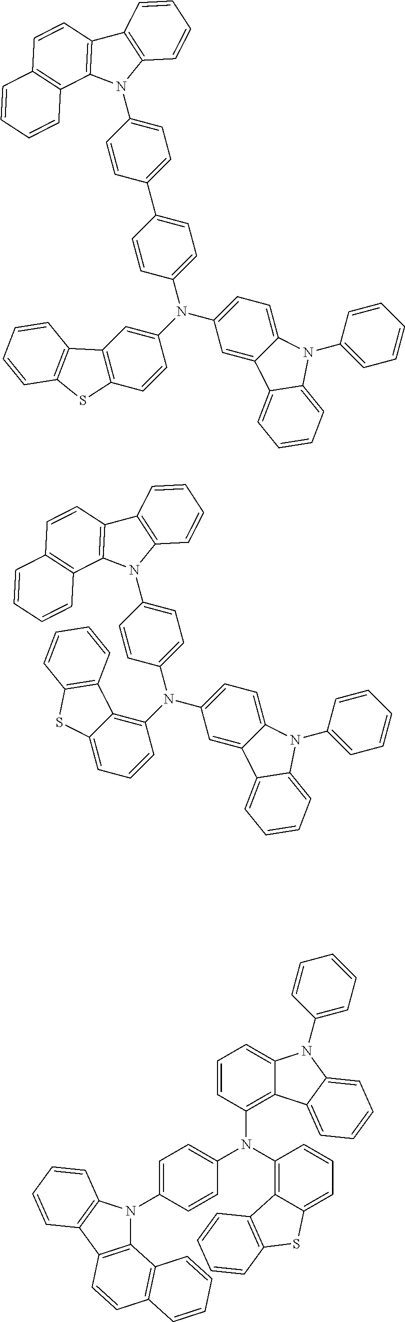 [SMILES: c1ccc(-n2c3ccccc3c3cc(N(c4ccc(-c5ccc(-n6c7ccccc7c7ccc8ccccc8c76)cc5)cc4)c4ccc5sc6ccccc6c5c4)ccc32)cc1 ; c1ccc(-n2c3ccccc3c3cc(N(c4ccc(-n5c6ccccc6c6ccc7ccccc7c65)cc4)c4cccc5sc6ccccc6c45)ccc32)cc1 ; c1ccc(-n2c3ccccc3c3c(N(c4ccc(-n5c6ccccc6c6ccc7ccccc7c65)cc4)c4cccc5sc6ccccc6c45)cccc32)cc1]

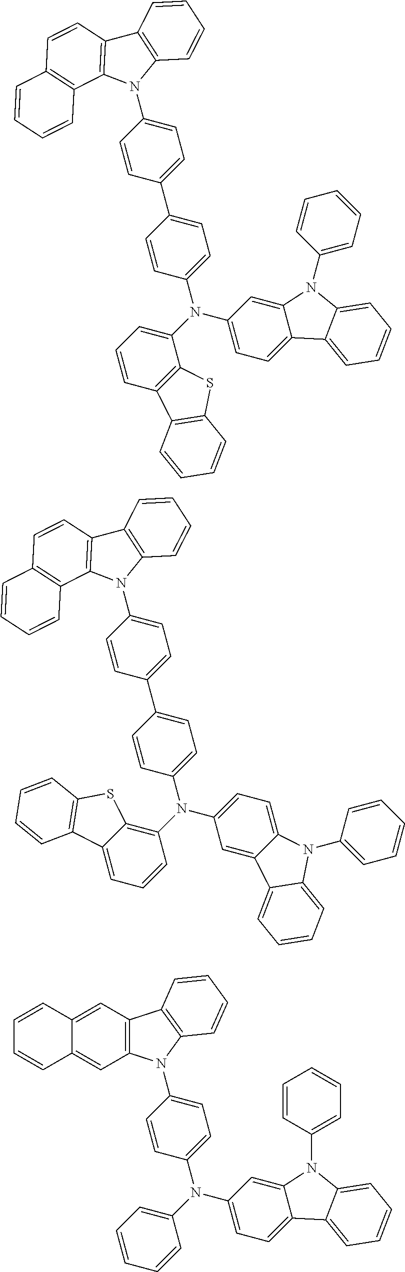

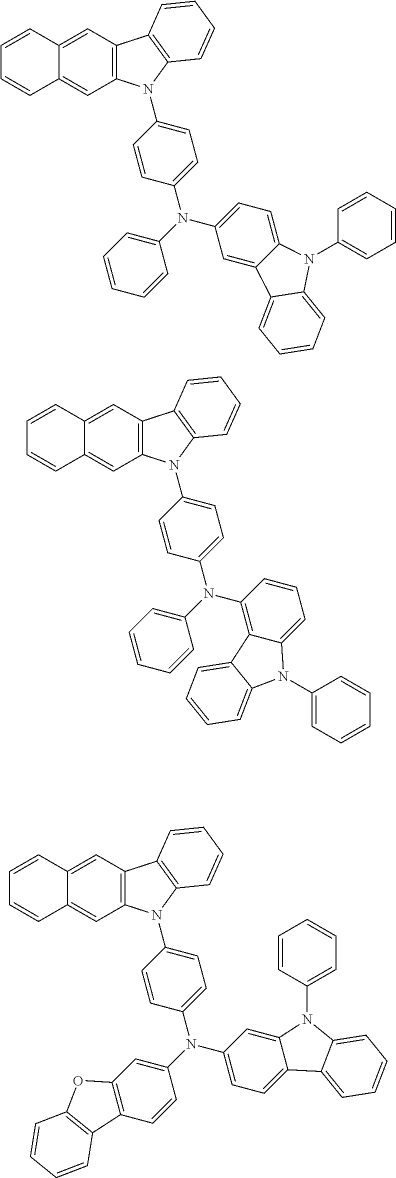 [SMILES: c1ccc(N(c2ccc(-n3c4ccccc4c4cc5ccccc5cc43)cc2)c2ccc3c(c2)c2ccccc2n3-c2ccccc2)cc1 ; c1ccc(N(c2ccc(-n3c4ccccc4c4cc5ccccc5cc43)cc2)c2cccc3c2c2ccccc2n3-c2ccccc2)cc1 ; c1ccc(-n2c3ccccc3c3ccc(N(c4ccc(-n5c6ccccc6c6cc7ccccc7cc65)cc4)c4ccc5c(c4)oc4ccccc45)cc32)cc1]

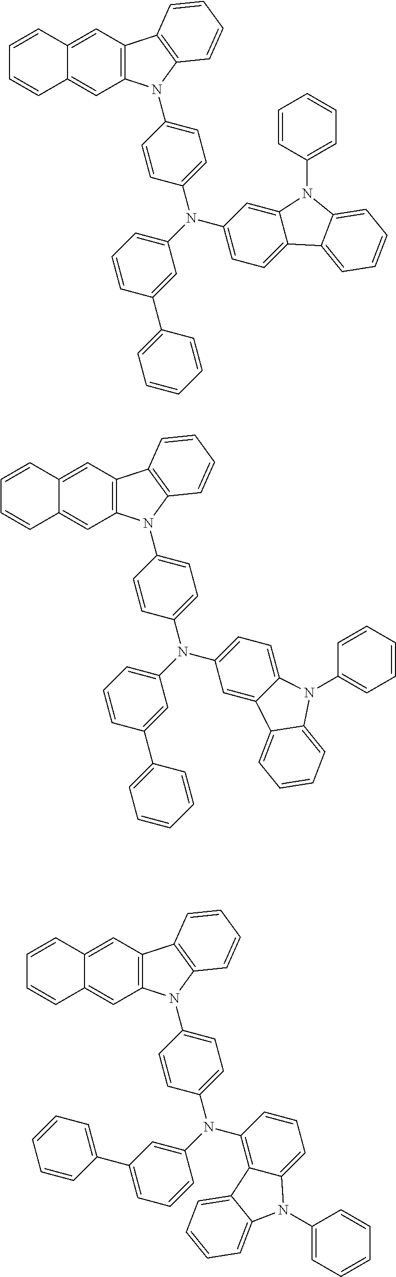

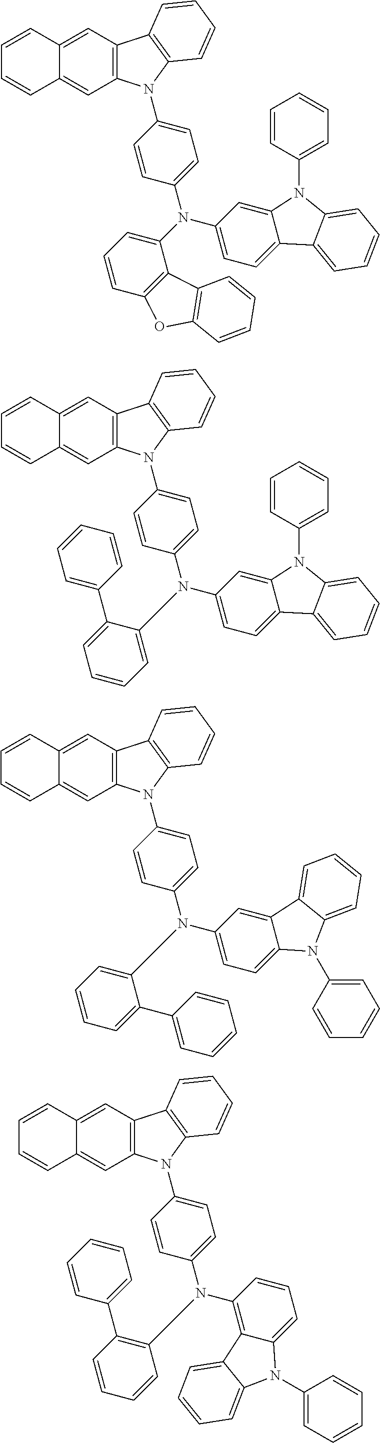

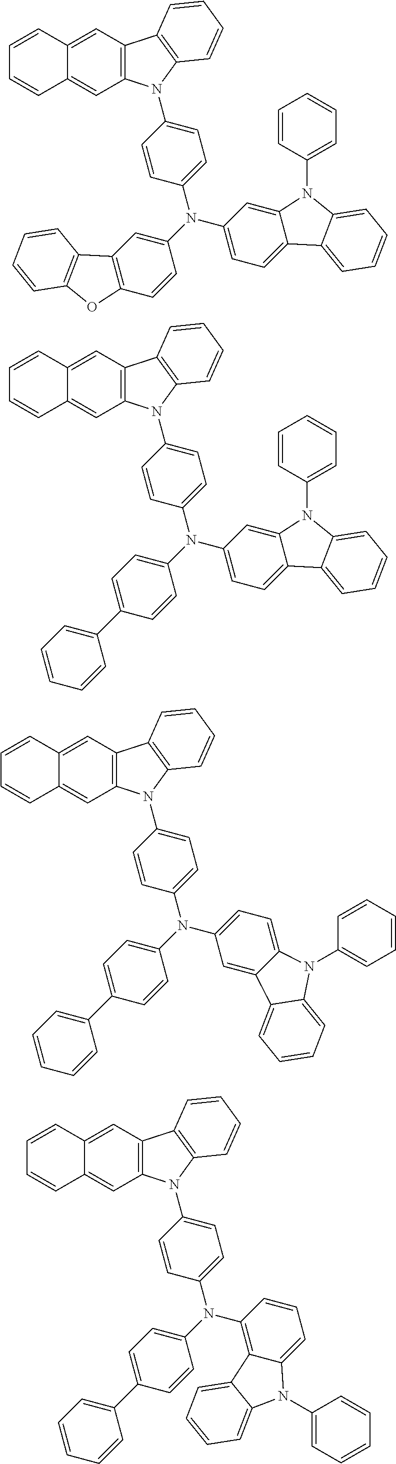

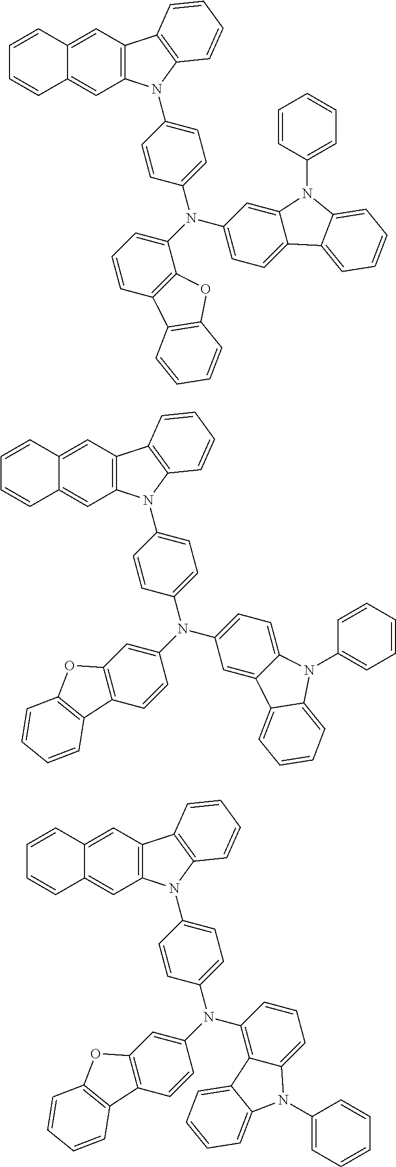

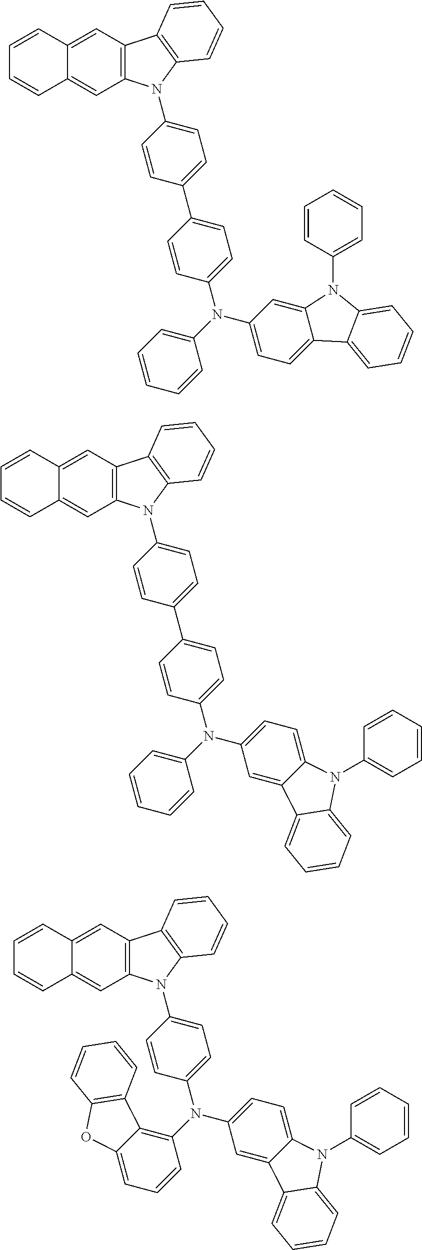

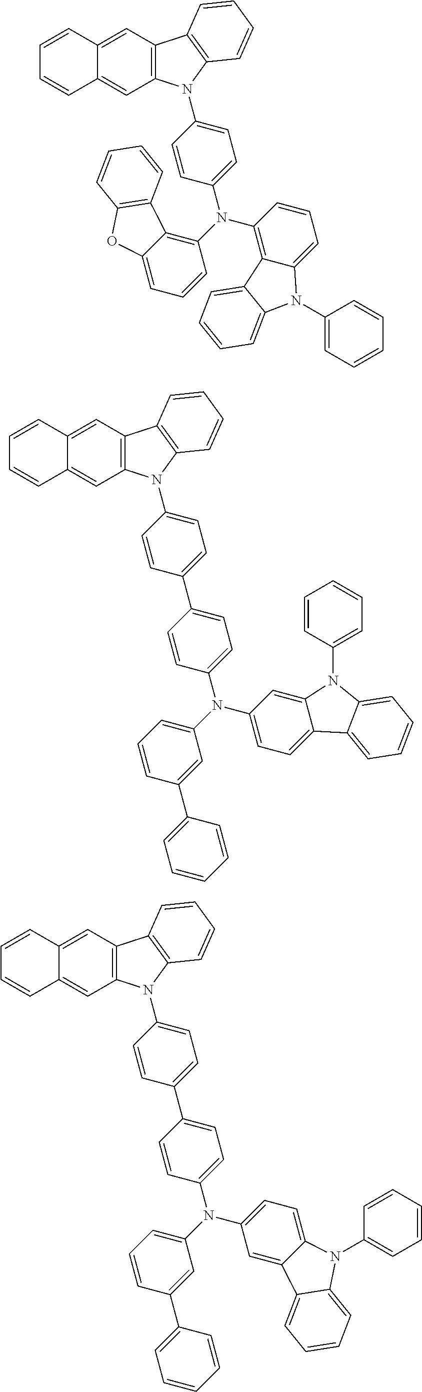

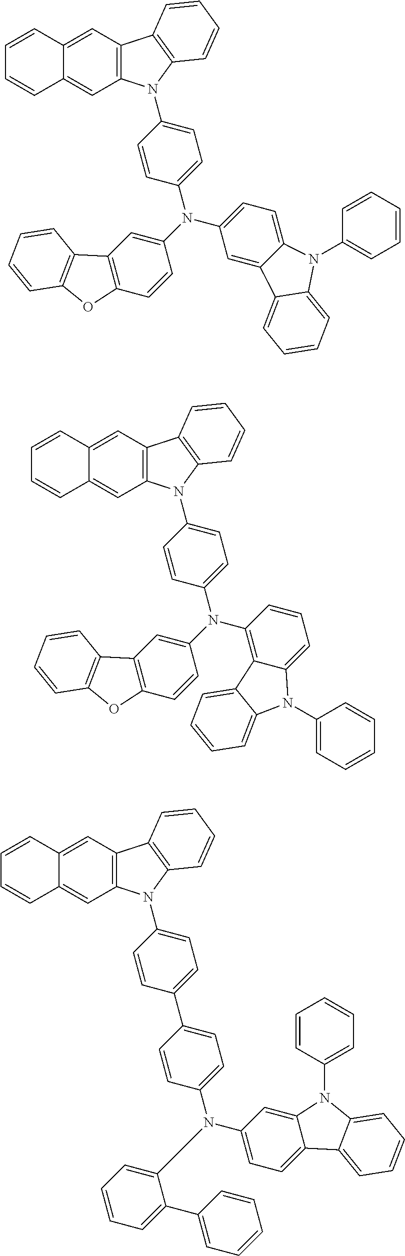 [SMILES: c1ccc(-n2c3ccccc3c3cc(N(c4ccc(-n5c6ccccc6c6cc7ccccc7cc65)cc4)c4ccc5oc6ccccc6c5c4)ccc32)cc1 ; c1ccc(-n2c3ccccc3c3c(N(c4ccc(-n5c6ccccc6c6cc7ccccc7cc65)cc4)c4ccc5oc6ccccc6c5c4)cccc32)cc1 ; c1ccc(-c2ccccc2N(c2ccc(-c3ccc(-n4c5ccccc5c5cc6ccccc6cc54)cc3)cc2)c2ccc3c4ccccc4n(-c4ccccc4)c3c2)cc1]

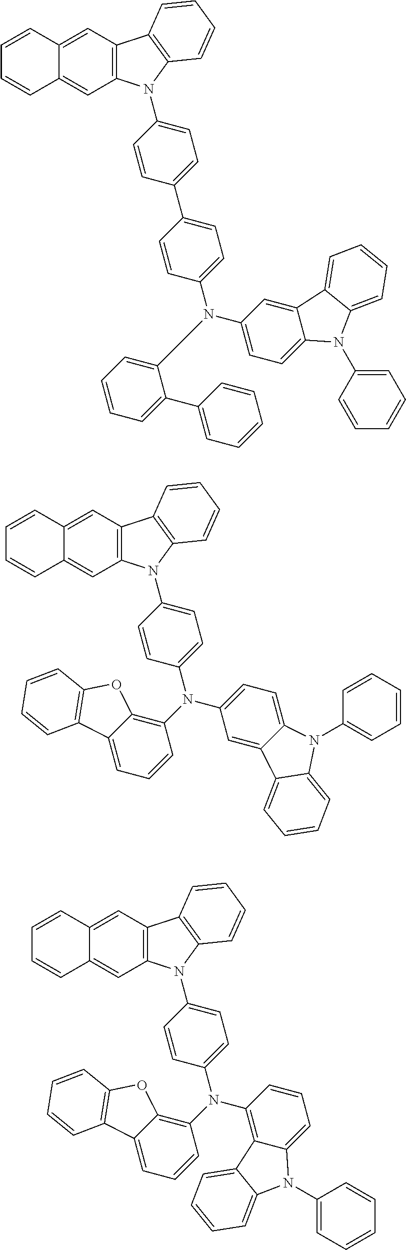

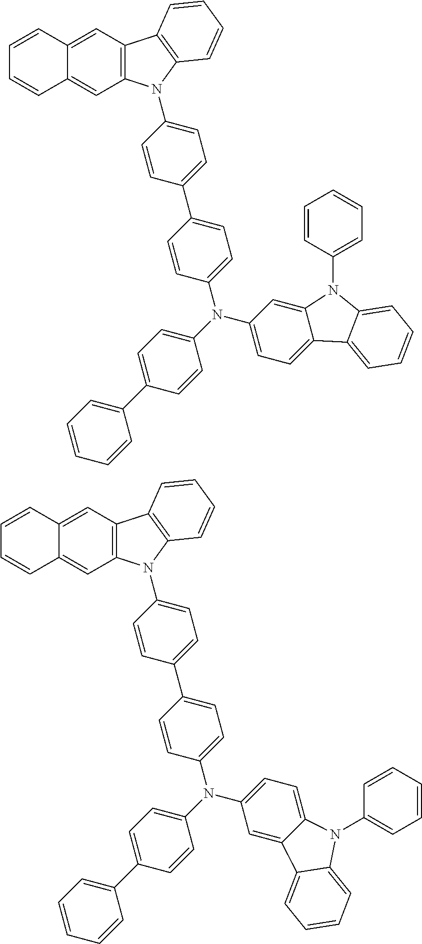 [SMILES: c1ccc(-c2ccc(N(c3ccc(-c4ccc(-n5c6ccccc6c6cc7ccccc7cc65)cc4)cc3)c3ccc4c5ccccc5n(-c5ccccc5)c4c3)cc2)cc1 ; c1ccc(-c2ccc(N(c3ccc(-c4ccc(-n5c6ccccc6c6cc7ccccc7cc65)cc4)cc3)c3ccc4c(c3)c3ccccc3n4-c3ccccc3)cc2)cc1]

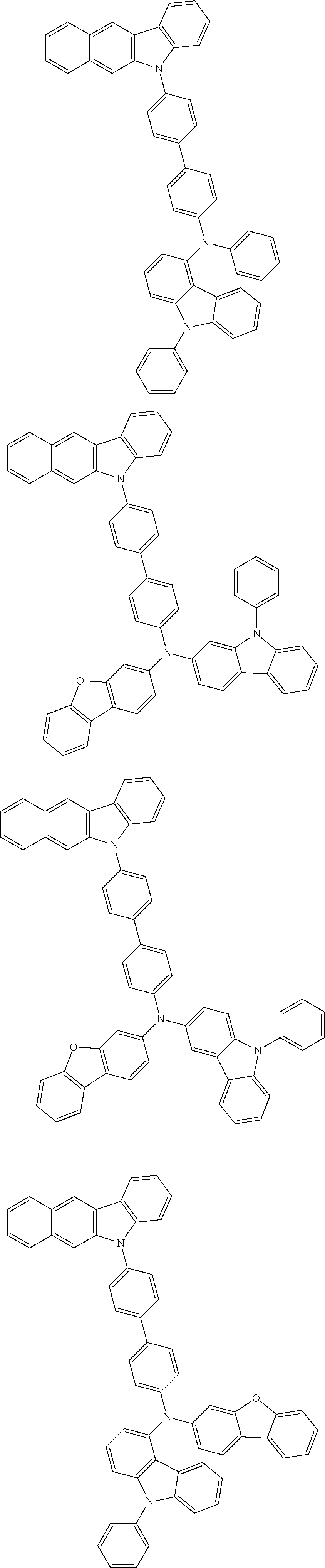

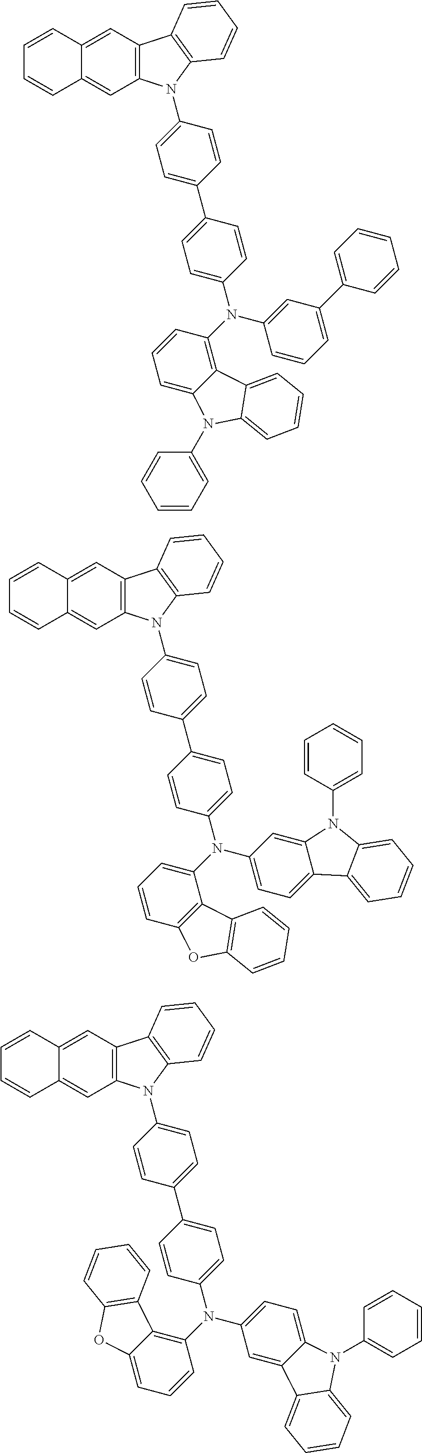

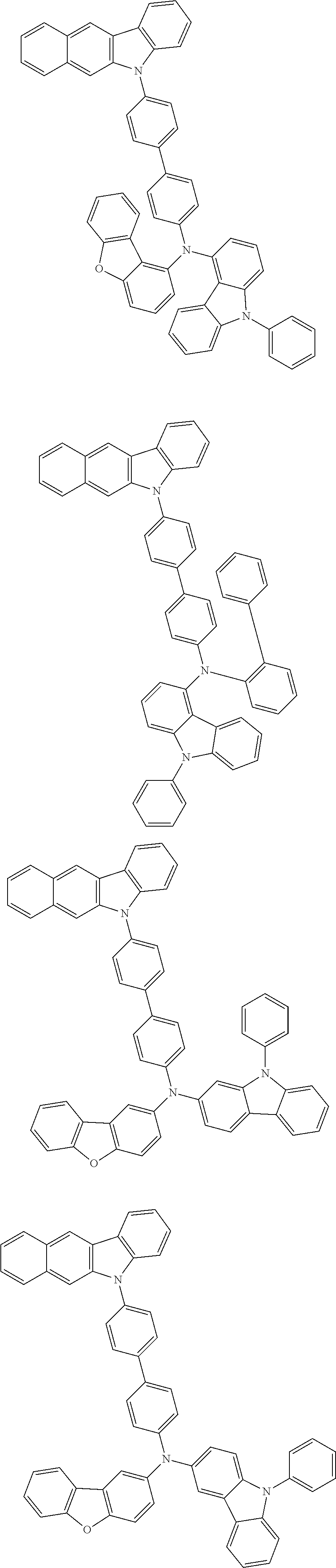

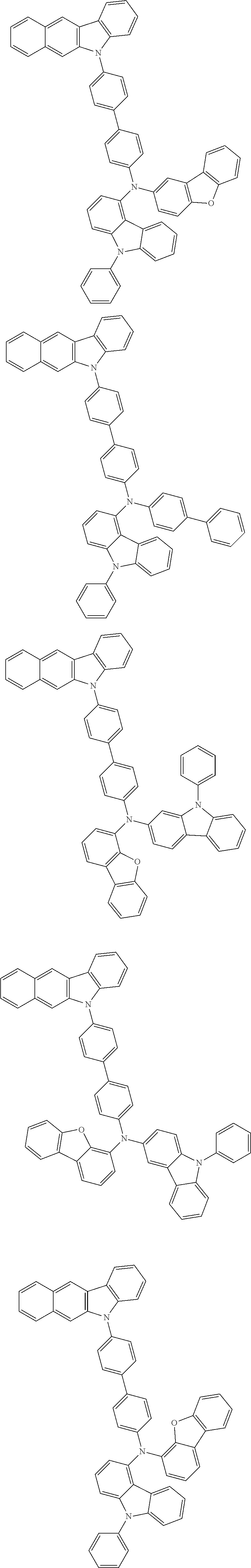

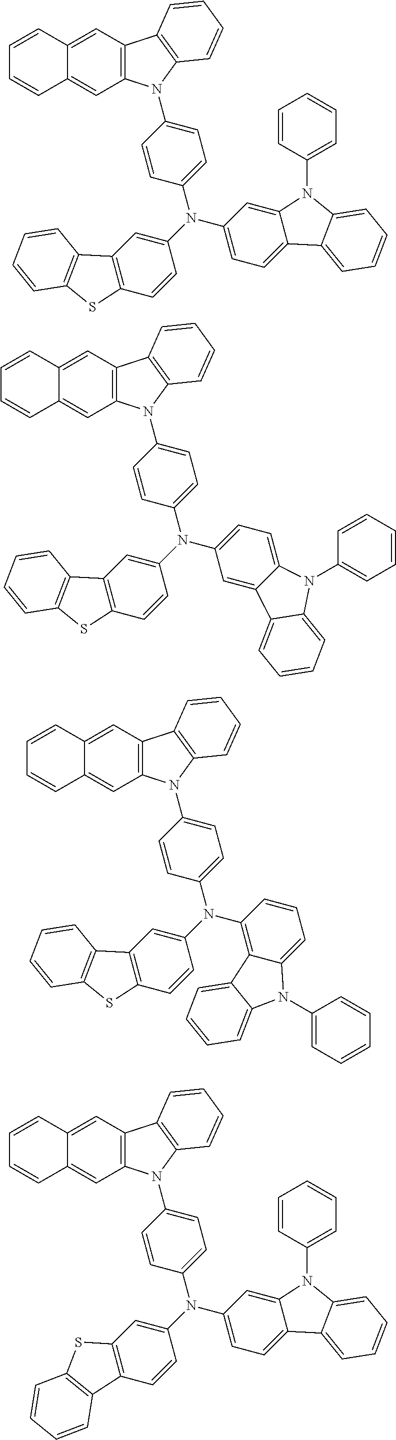

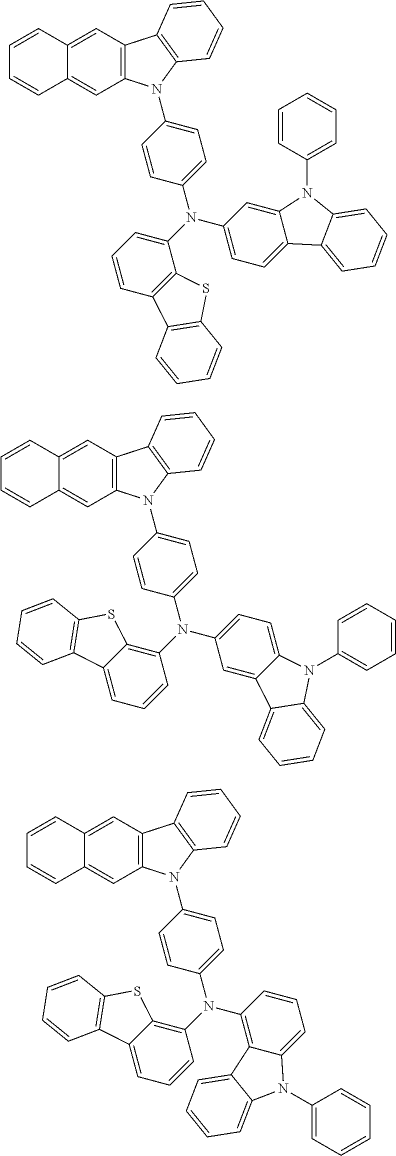

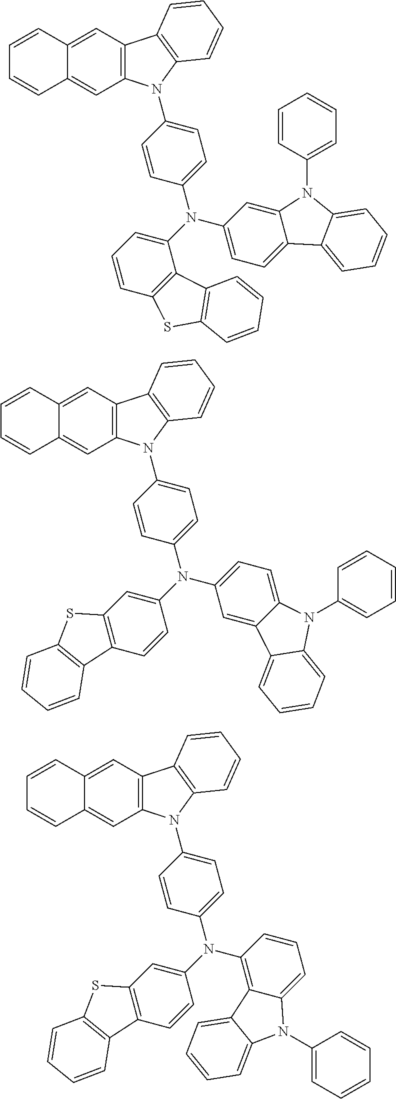

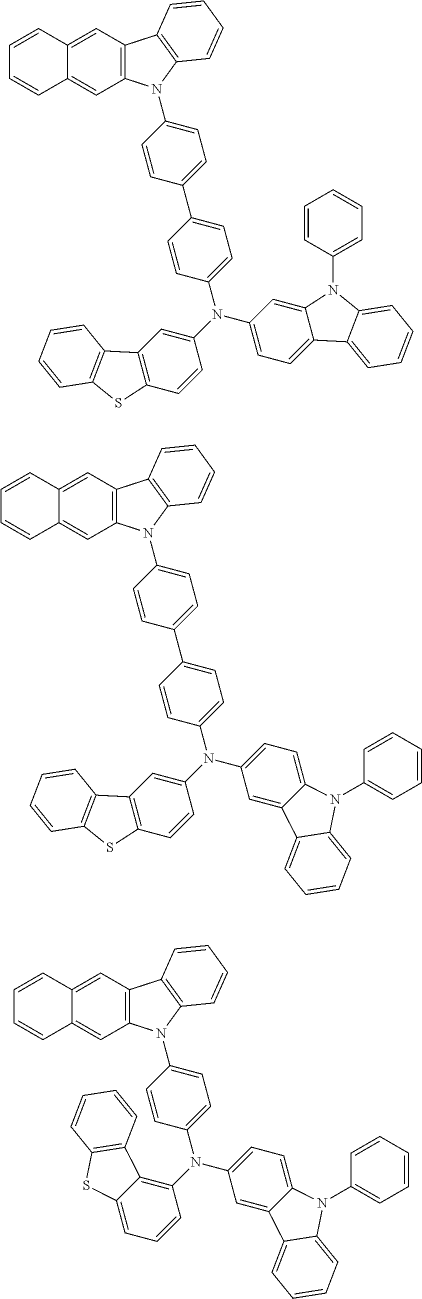 [SMILES: c1ccc(-n2c3ccccc3c3ccc(N(c4ccc(-c5ccc(-n6c7ccccc7c7cc8ccccc8cc76)cc5)cc4)c4ccc5sc6ccccc6c5c4)cc32)cc1 ; c1ccc(-n2c3ccccc3c3cc(N(c4ccc(-c5ccc(-n6c7ccccc7c7cc8ccccc8cc76)cc5)cc4)c4ccc5sc6ccccc6c5c4)ccc32)cc1 ; c1ccc(-n2c3ccccc3c3cc(N(c4ccc(-n5c6ccccc6c6cc7ccccc7cc65)cc4)c4cccc5sc6ccccc6c45)ccc32)cc1]

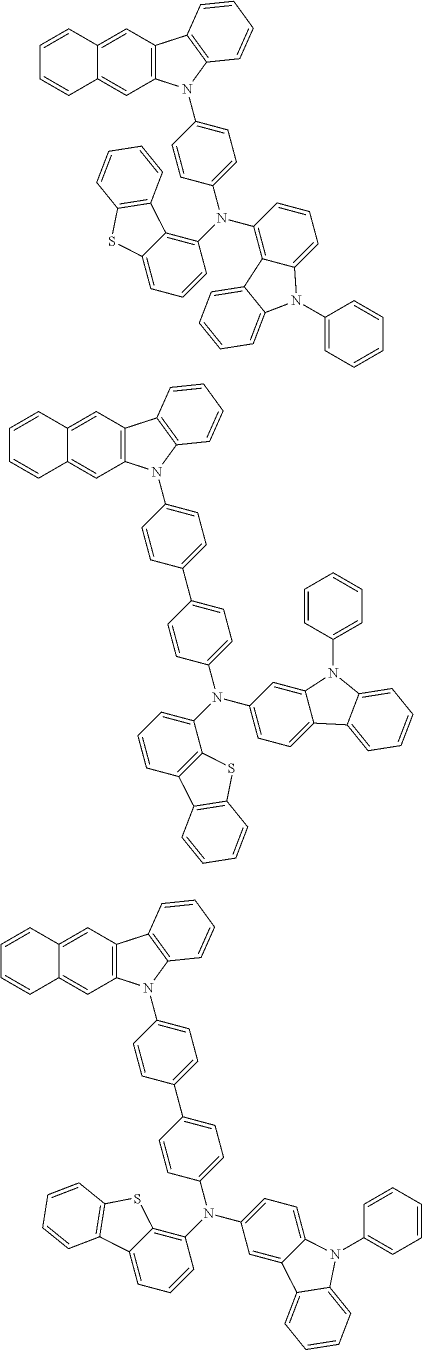

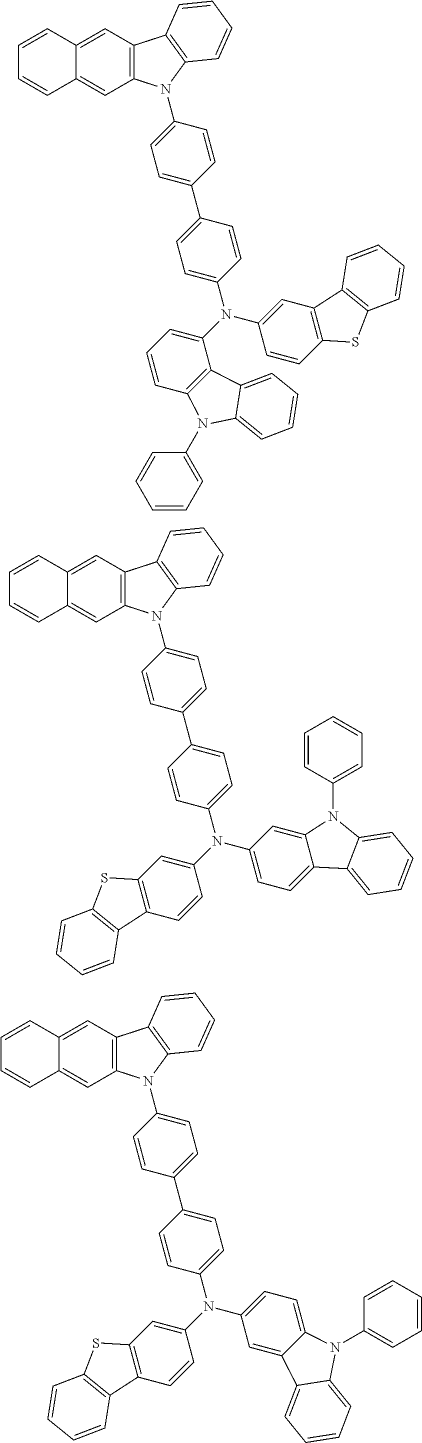

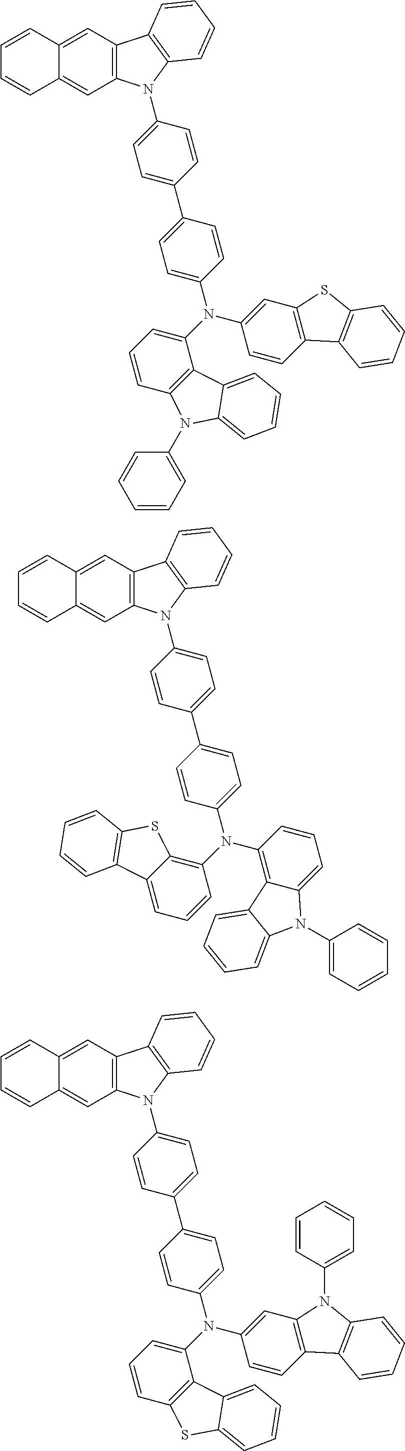

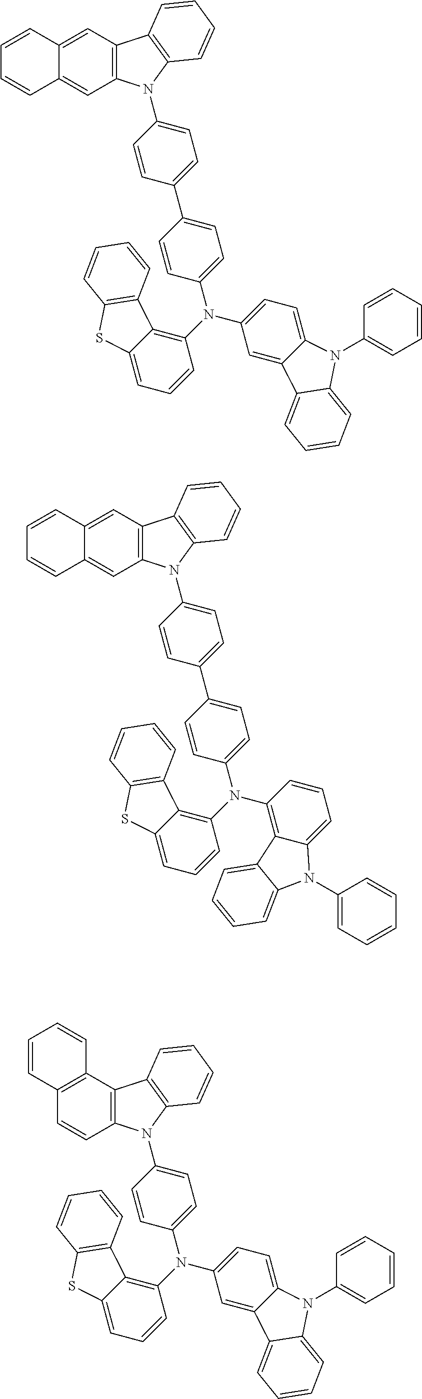 [SMILES: c1ccc(-n2c3ccccc3c3cc(N(c4ccc(-c5ccc(-n6c7ccccc7c7cc8ccccc8cc76)cc5)cc4)c4cccc5sc6ccccc6c45)ccc32)cc1 ; c1ccc(-n2c3ccccc3c3c(N(c4ccc(-c5ccc(-n6c7ccccc7c7cc8ccccc8cc76)cc5)cc4)c4cccc5sc6ccccc6c45)cccc32)cc1 ; c1ccc(-n2c3ccccc3c3cc(N(c4ccc(-n5c6ccccc6c6c7ccccc7ccc65)cc4)c4cccc5sc6ccccc6c45)ccc32)cc1]

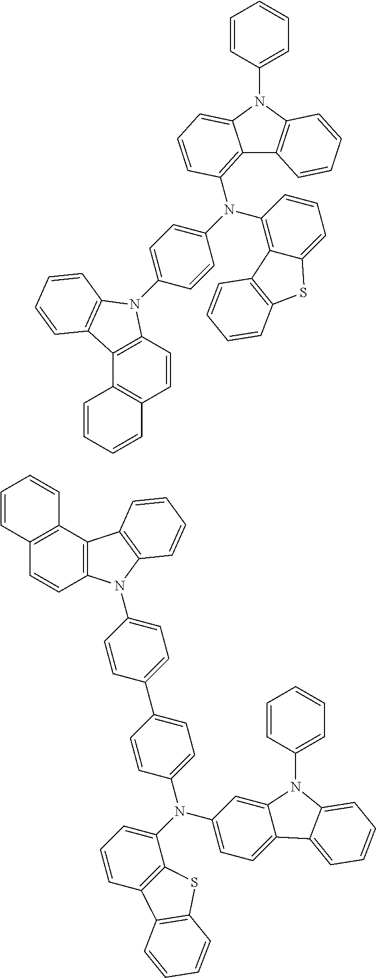 [SMILES: c1ccc(-n2c3ccccc3c3c(N(c4ccc(-n5c6ccccc6c6c7ccccc7ccc65)cc4)c4cccc5sc6ccccc6c45)cccc32)cc1 ; c1ccc(-n2c3ccccc3c3ccc(N(c4ccc(-c5ccc(-n6c7ccccc7c7c8ccccc8ccc76)cc5)cc4)c4cccc5c4sc4ccccc45)cc32)cc1]

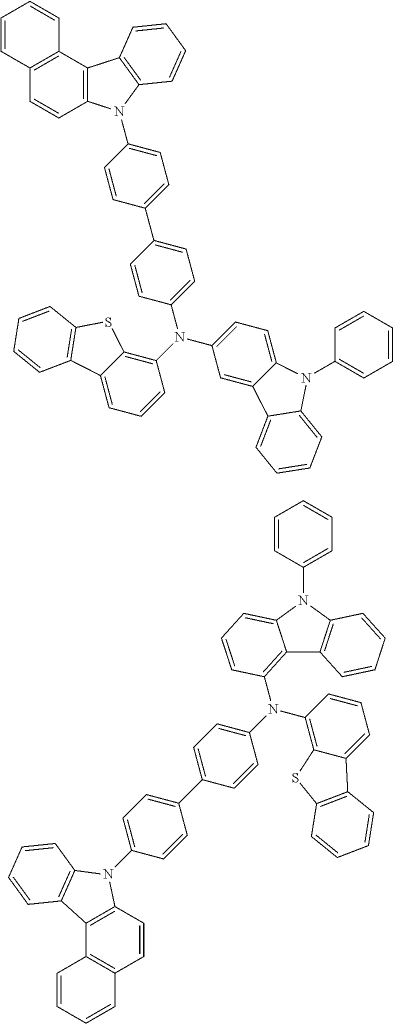

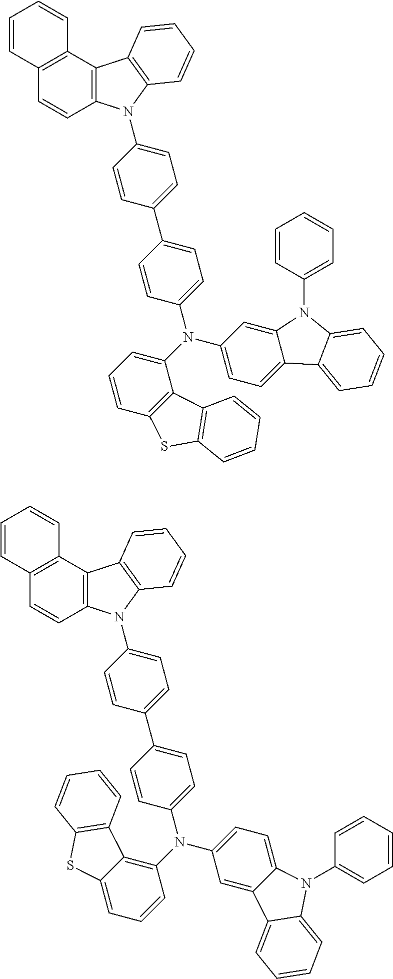 [SMILES: c1ccc(-n2c3ccccc3c3ccc(N(c4ccc(-c5ccc(-n6c7ccccc7c7c8ccccc8ccc76)cc5)cc4)c4cccc5sc6ccccc6c45)cc32)cc1 ; c1ccc(-n2c3ccccc3c3cc(N(c4ccc(-c5ccc(-n6c7ccccc7c7c8ccccc8ccc76)cc5)cc4)c4cccc5sc6ccccc6c45)ccc32)cc1]

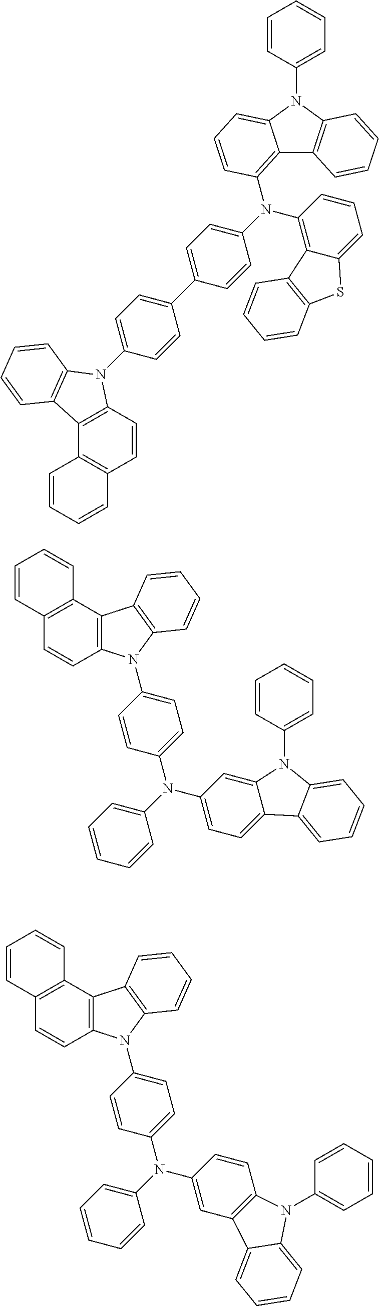

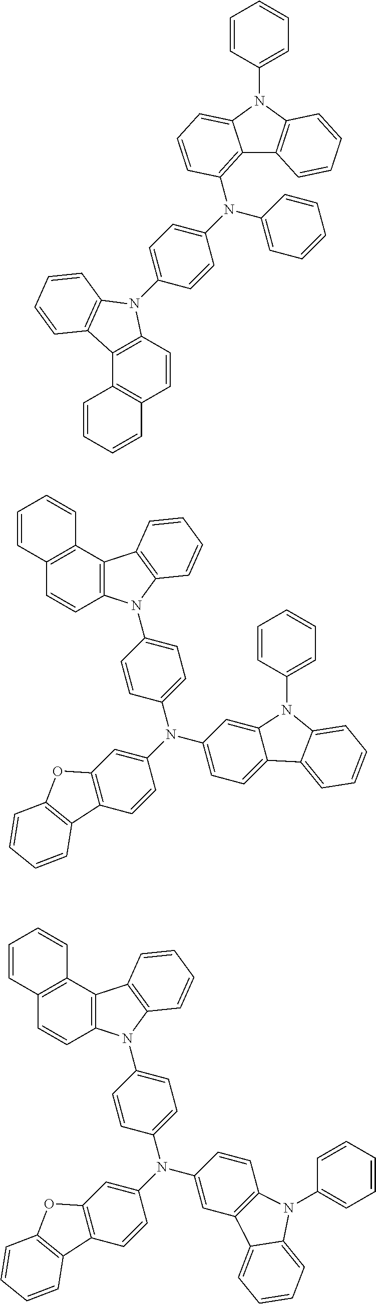 [SMILES: c1ccc(N(c2ccc(-n3c4ccccc4c4c5ccccc5ccc43)cc2)c2cccc3c2c2ccccc2n3-c2ccccc2)cc1 ; c1ccc(-n2c3ccccc3c3ccc(N(c4ccc(-n5c6ccccc6c6c7ccccc7ccc65)cc4)c4ccc5c(c4)oc4ccccc45)cc32)cc1 ; c1ccc(-n2c3ccccc3c3cc(N(c4ccc(-n5c6ccccc6c6c7ccccc7ccc65)cc4)c4ccc5c(c4)oc4ccccc45)ccc32)cc1]

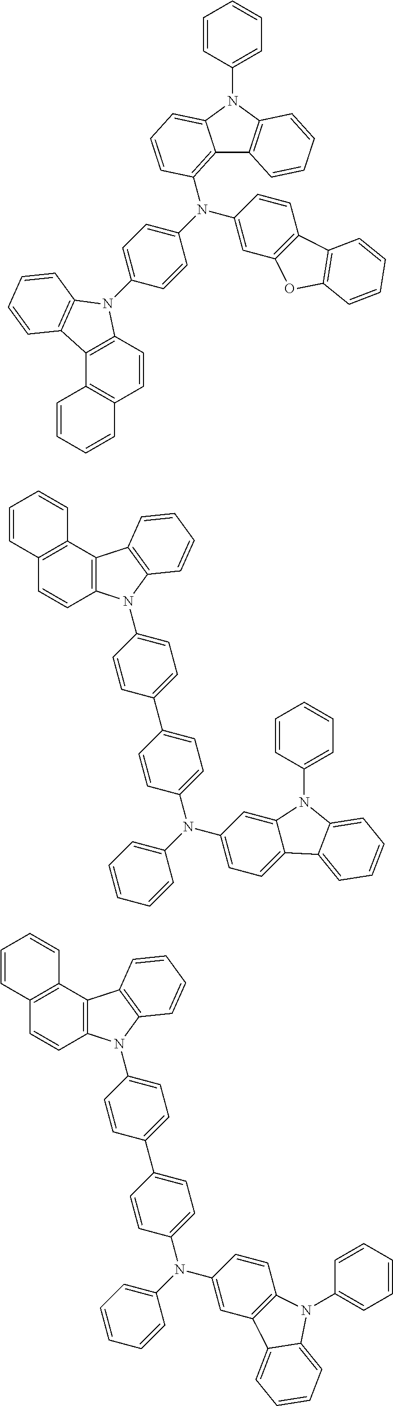 [SMILES: c1ccc(-n2c3ccccc3c3c(N(c4ccc(-n5c6ccccc6c6c7ccccc7ccc65)cc4)c4ccc5c(c4)oc4ccccc45)cccc32)cc1 ; c1ccc(N(c2ccc(-c3ccc(-n4c5ccccc5c5c6ccccc6ccc54)cc3)cc2)c2ccc3c4ccccc4n(-c4ccccc4)c3c2)cc1 ; c1ccc(N(c2ccc(-c3ccc(-n4c5ccccc5c5c6ccccc6ccc54)cc3)cc2)c2ccc3c(c2)c2ccccc2n3-c2ccccc2)cc1]

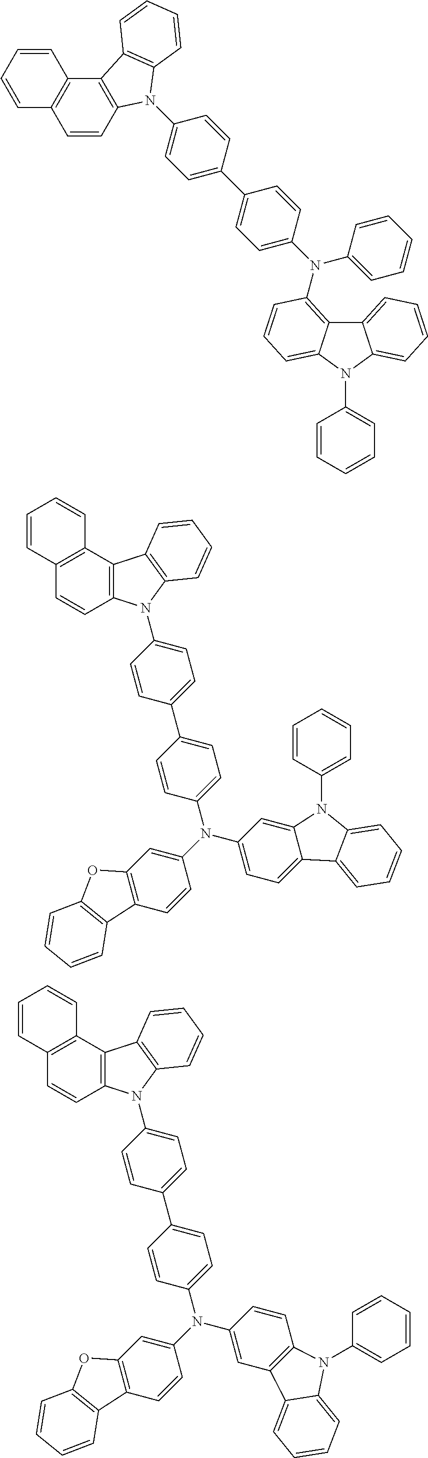

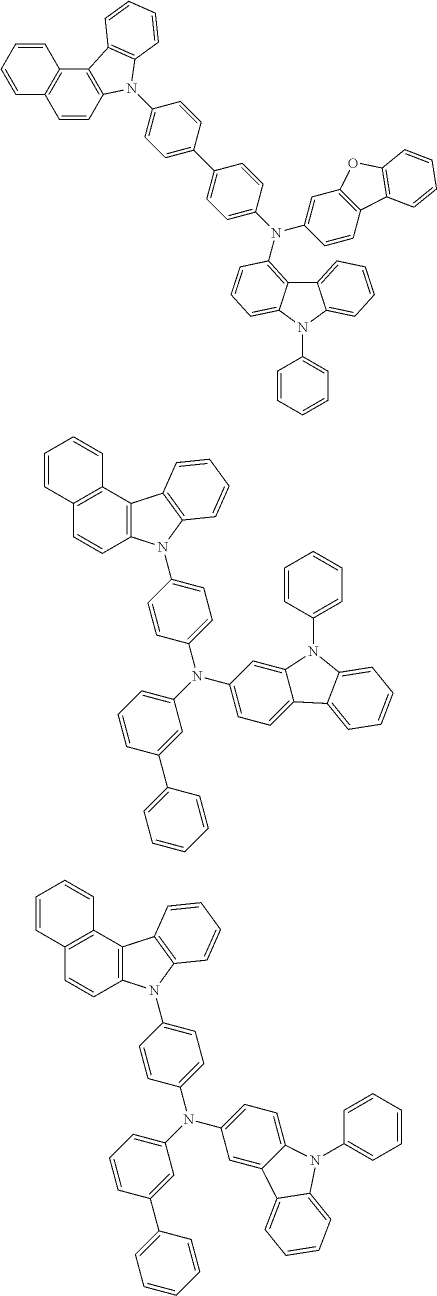 [SMILES: c1ccc(-n2c3ccccc3c3c(N(c4ccc(-c5ccc(-n6c7ccccc7c7c8ccccc8ccc76)cc5)cc4)c4ccc5c(c4)oc4ccccc45)cccc32)cc1 ; c1ccc(-c2cccc(N(c3ccc(-n4c5ccccc5c5c6ccccc6ccc54)cc3)c3ccc4c5ccccc5n(-c5ccccc5)c4c3)c2)cc1 ; c1ccc(-c2cccc(N(c3ccc(-n4c5ccccc5c5c6ccccc6ccc54)cc3)c3ccc4c(c3)c3ccccc3n4-c3ccccc3)c2)cc1]

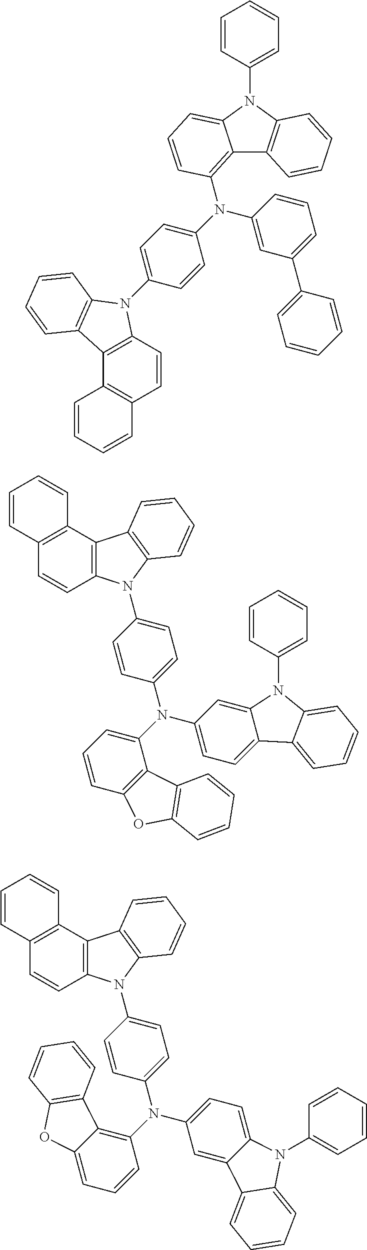 [SMILES: c1ccc(-c2cccc(N(c3ccc(-n4c5ccccc5c5c6ccccc6ccc54)cc3)c3cccc4c3c3ccccc3n4-c3ccccc3)c2)cc1 ; c1ccc(-n2c3ccccc3c3ccc(N(c4ccc(-n5c6ccccc6c6c7ccccc7ccc65)cc4)c4cccc5oc6ccccc6c45)cc32)cc1 ; c1ccc(-n2c3ccccc3c3cc(N(c4ccc(-n5c6ccccc6c6c7ccccc7ccc65)cc4)c4cccc5oc6ccccc6c45)ccc32)cc1]

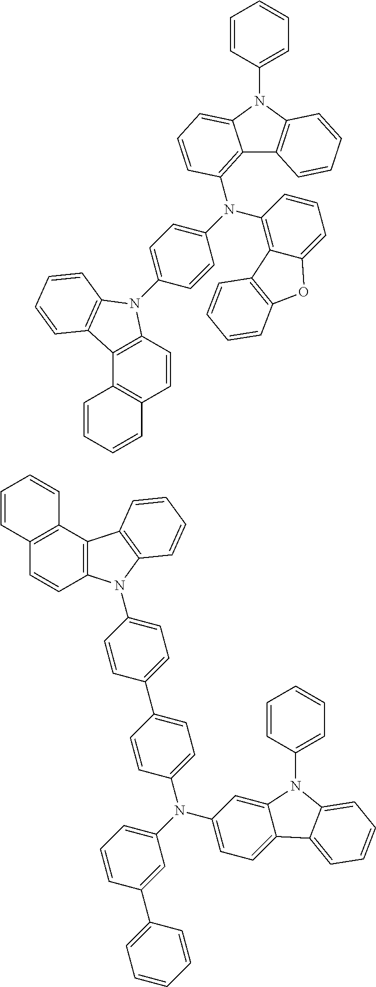 [SMILES: c1ccc(-n2c3ccccc3c3c(N(c4ccc(-n5c6ccccc6c6c7ccccc7ccc65)cc4)c4cccc5oc6ccccc6c45)cccc32)cc1 ; c1ccc(-c2cccc(N(c3ccc(-c4ccc(-n5c6ccccc6c6c7ccccc7ccc65)cc4)cc3)c3ccc4c5ccccc5n(-c5ccccc5)c4c3)c2)cc1]

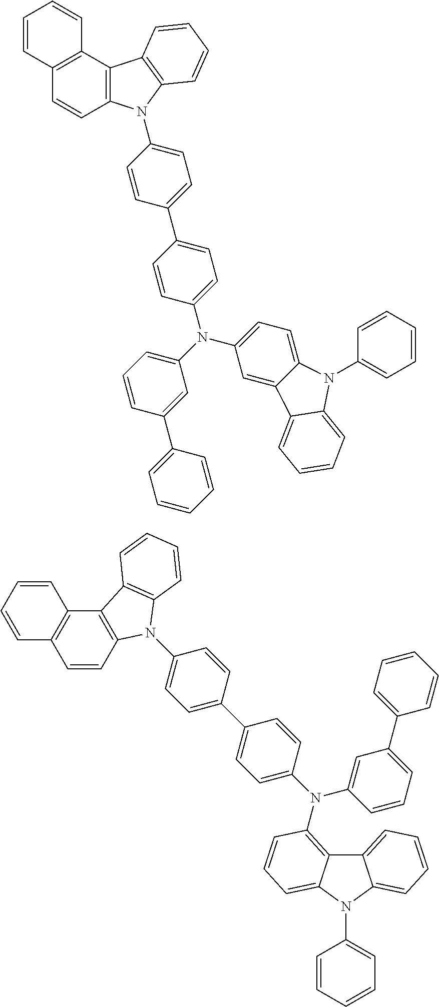

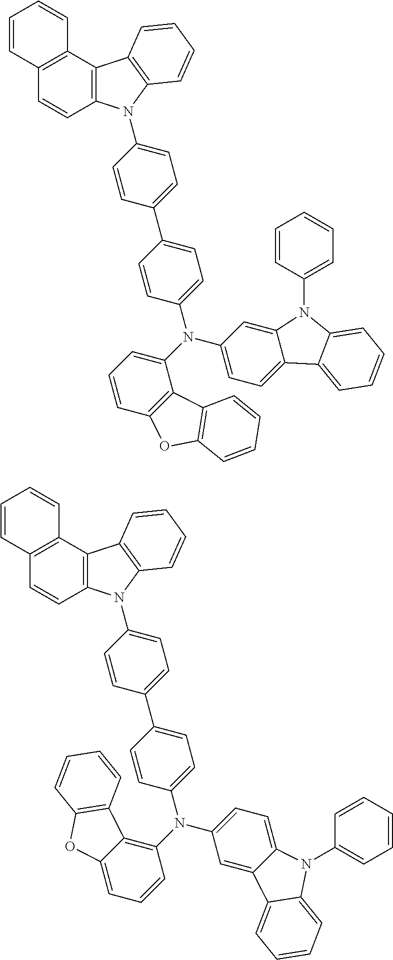 [SMILES: c1ccc(-n2c3ccccc3c3ccc(N(c4ccc(-c5ccc(-n6c7ccccc7c7c8ccccc8ccc76)cc5)cc4)c4cccc5oc6ccccc6c45)cc32)cc1 ; c1ccc(-n2c3ccccc3c3cc(N(c4ccc(-c5ccc(-n6c7ccccc7c7c8ccccc8ccc76)cc5)cc4)c4cccc5oc6ccccc6c45)ccc32)cc1]

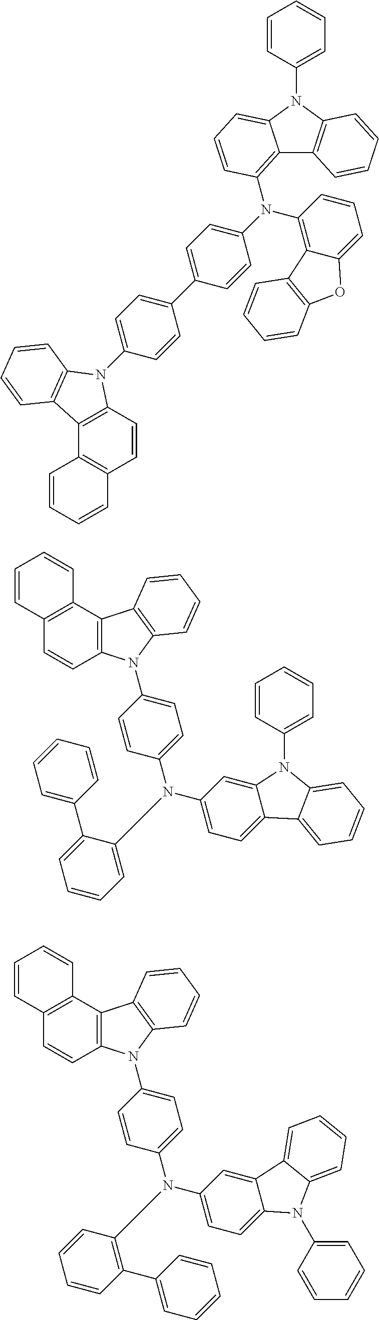

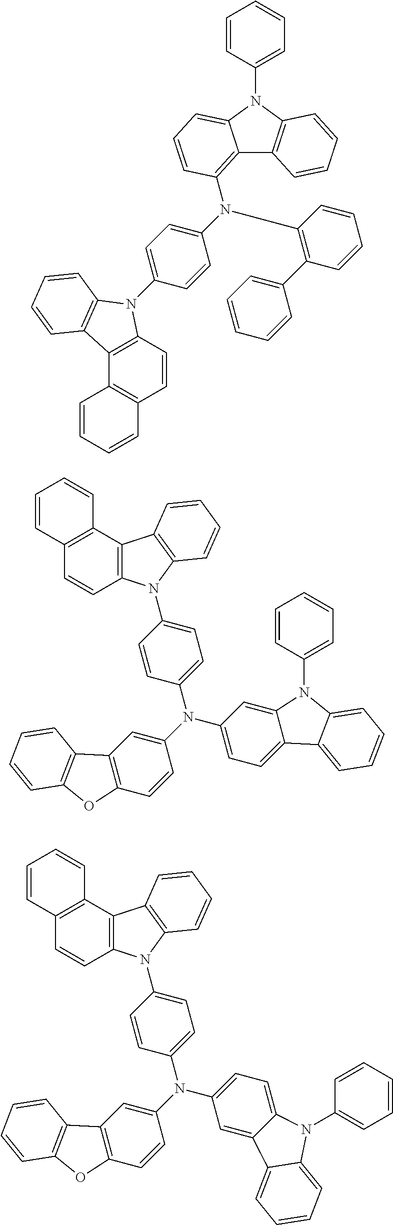

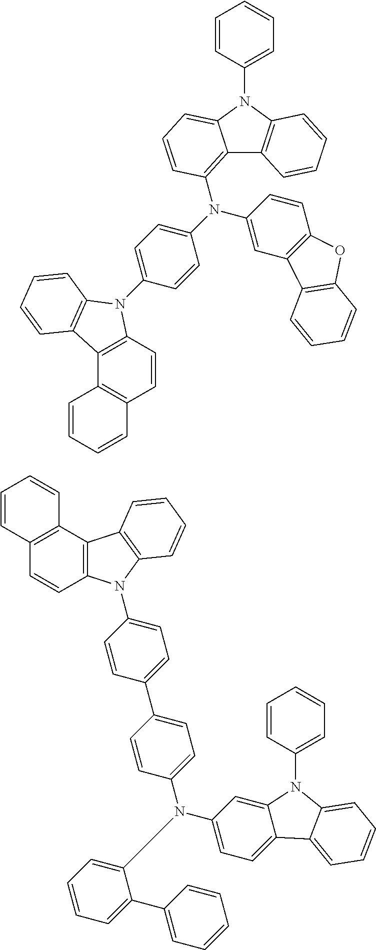 [SMILES: c1ccc(-n2c3ccccc3c3c(N(c4ccc(-n5c6ccccc6c6c7ccccc7ccc65)cc4)c4ccc5oc6ccccc6c5c4)cccc32)cc1 ; c1ccc(-c2ccccc2N(c2ccc(-c3ccc(-n4c5ccccc5c5c6ccccc6ccc54)cc3)cc2)c2ccc3c4ccccc4n(-c4ccccc4)c3c2)cc1]

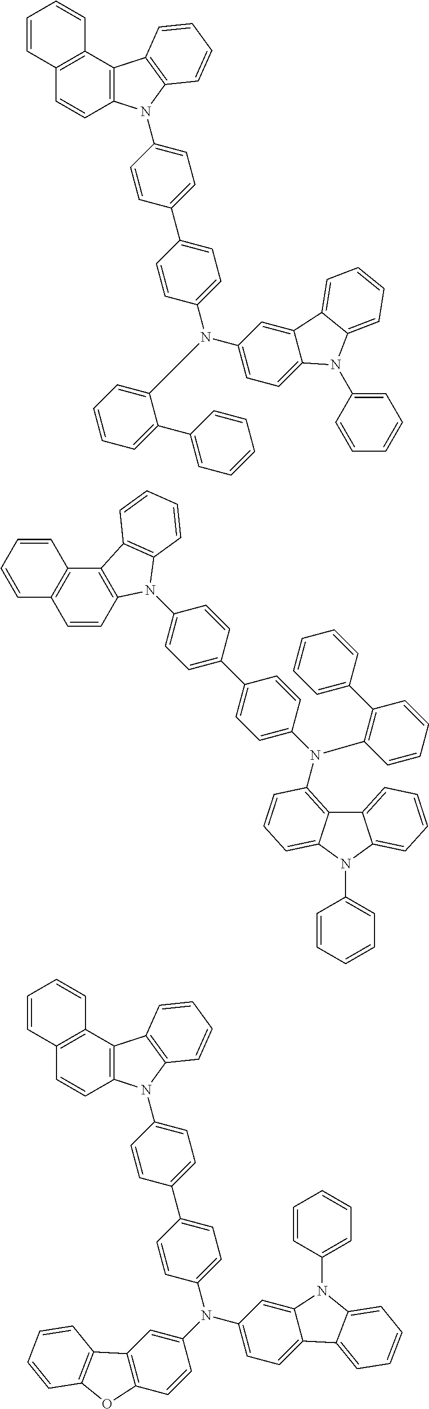

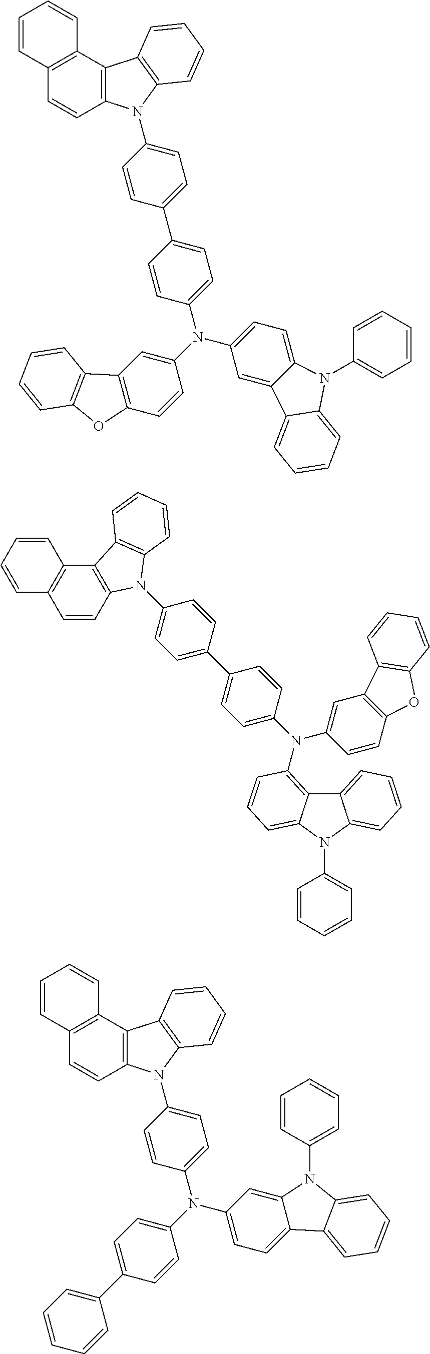 [SMILES: c1ccc(-n2c3ccccc3c3cc(N(c4ccc(-c5ccc(-n6c7ccccc7c7c8ccccc8ccc76)cc5)cc4)c4ccc5oc6ccccc6c5c4)ccc32)cc1 ; c1ccc(-n2c3ccccc3c3c(N(c4ccc(-c5ccc(-n6c7ccccc7c7c8ccccc8ccc76)cc5)cc4)c4ccc5oc6ccccc6c5c4)cccc32)cc1 ; c1ccc(-c2ccc(N(c3ccc(-n4c5ccccc5c5c6ccccc6ccc54)cc3)c3ccc4c5ccccc5n(-c5ccccc5)c4c3)cc2)cc1]

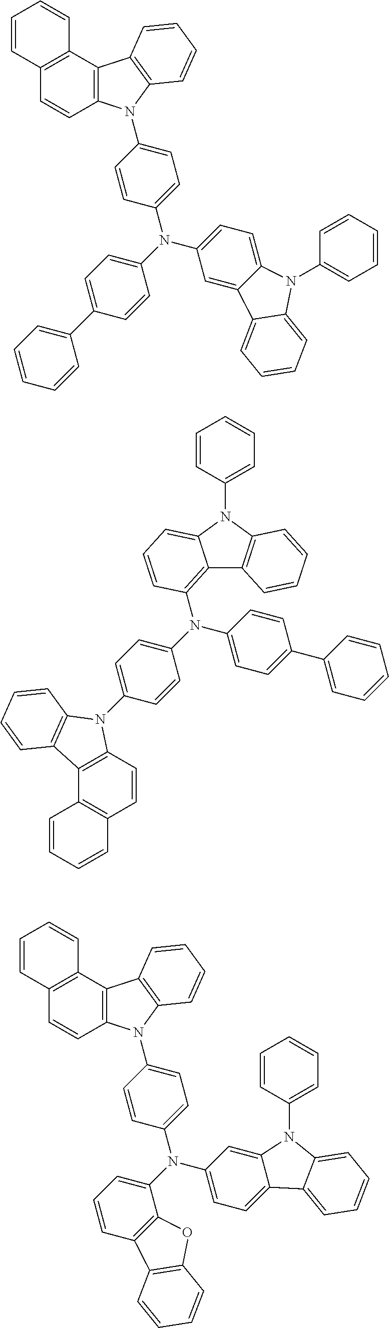 [SMILES: c1ccc(-c2ccc(N(c3ccc(-n4c5ccccc5c5c6ccccc6ccc54)cc3)c3ccc4c(c3)c3ccccc3n4-c3ccccc3)cc2)cc1 ; c1ccc(-c2ccc(N(c3ccc(-n4c5ccccc5c5c6ccccc6ccc54)cc3)c3cccc4c3c3ccccc3n4-c3ccccc3)cc2)cc1 ; c1ccc(-n2c3ccccc3c3ccc(N(c4ccc(-n5c6ccccc6c6c7ccccc7ccc65)cc4)c4cccc5c4oc4ccccc45)cc32)cc1]

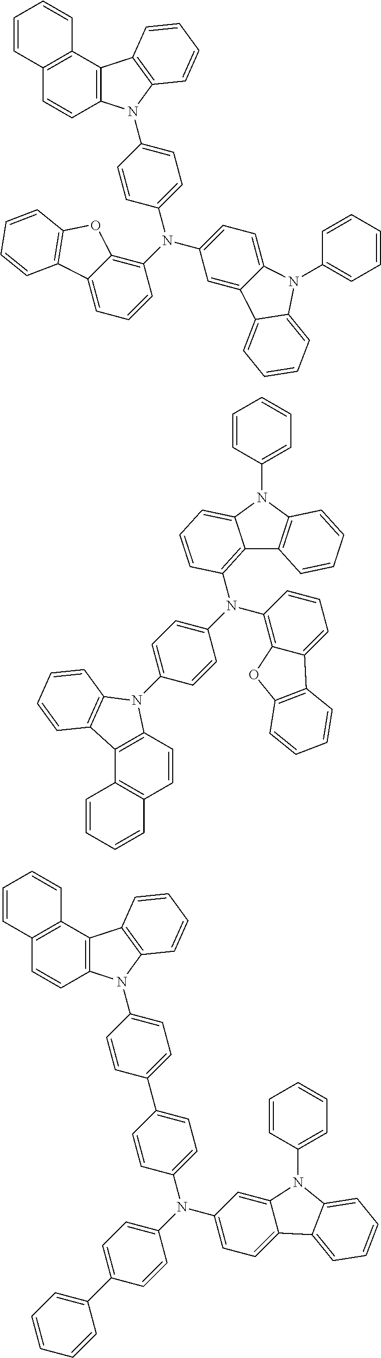

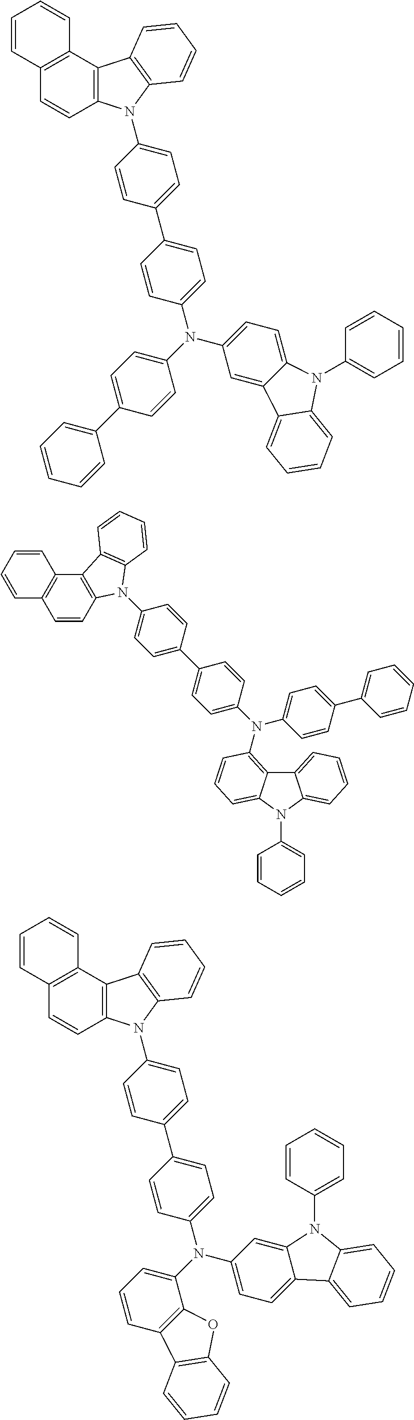

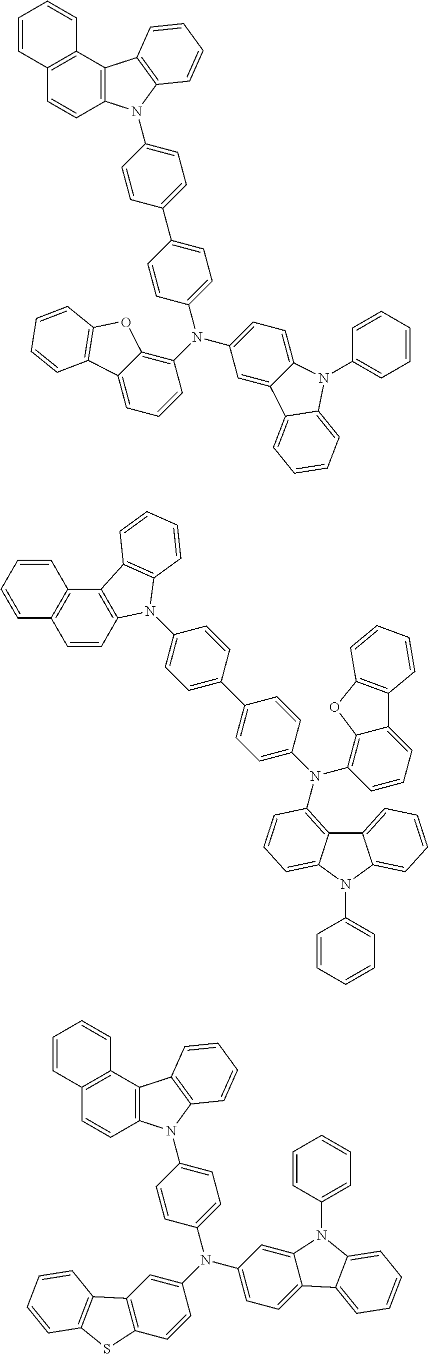 [SMILES: c1ccc(-n2c3ccccc3c3cc(N(c4ccc(-c5ccc(-n6c7ccccc7c7c8ccccc8ccc76)cc5)cc4)c4cccc5c4oc4ccccc45)ccc32)cc1 ; c1ccc(-n2c3ccccc3c3c(N(c4ccc(-c5ccc(-n6c7ccccc7c7c8ccccc8ccc76)cc5)cc4)c4cccc5c4oc4ccccc45)cccc32)cc1 ; c1ccc(-n2c3ccccc3c3ccc(N(c4ccc(-n5c6ccccc6c6c7ccccc7ccc65)cc4)c4ccc5sc6ccccc6c5c4)cc32)cc1]

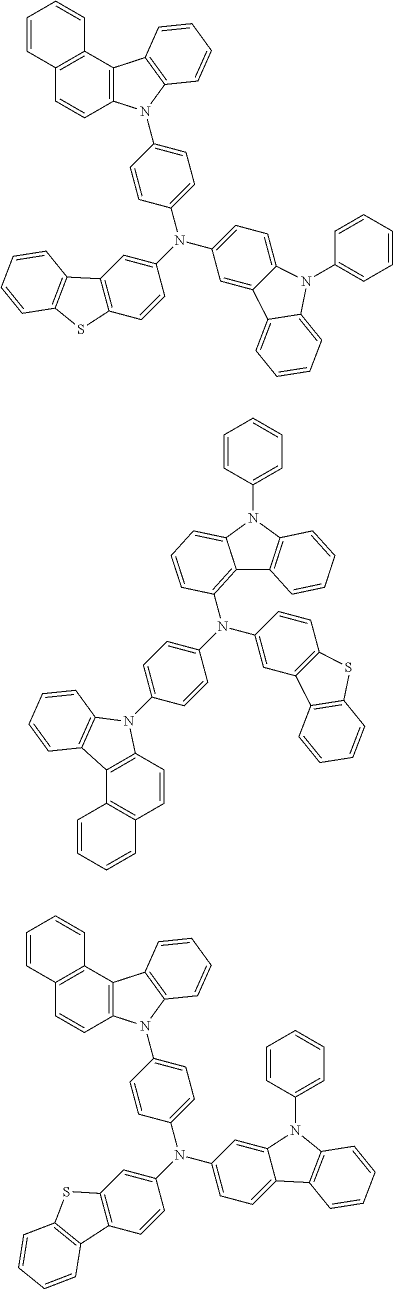 [SMILES: c1ccc(-n2c3ccccc3c3cc(N(c4ccc(-n5c6ccccc6c6c7ccccc7ccc65)cc4)c4ccc5sc6ccccc6c5c4)ccc32)cc1 ; c1ccc(-n2c3ccccc3c3c(N(c4ccc(-n5c6ccccc6c6c7ccccc7ccc65)cc4)c4ccc5sc6ccccc6c5c4)cccc32)cc1 ; c1ccc(-n2c3ccccc3c3ccc(N(c4ccc(-n5c6ccccc6c6c7ccccc7ccc65)cc4)c4ccc5c(c4)sc4ccccc45)cc32)cc1]

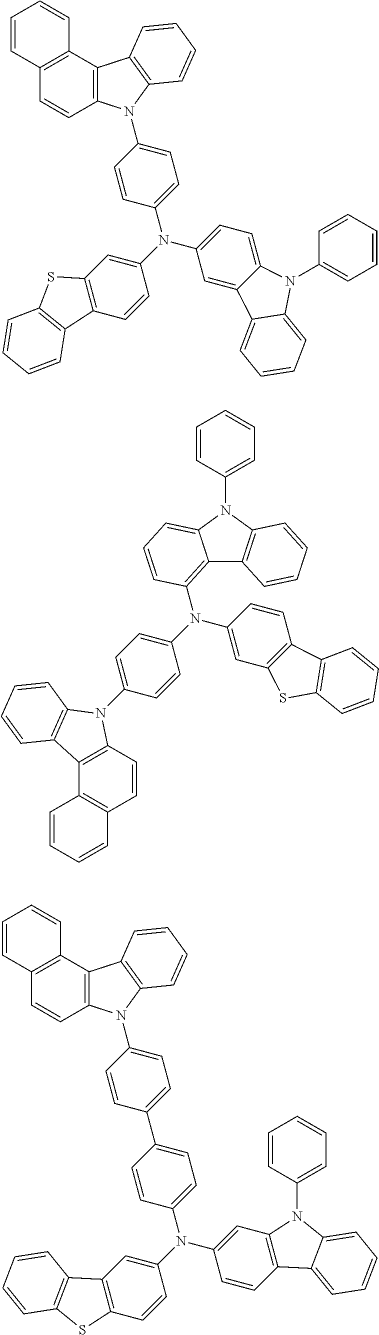 [SMILES: c1ccc(-n2c3ccccc3c3cc(N(c4ccc(-n5c6ccccc6c6c7ccccc7ccc65)cc4)c4ccc5c(c4)sc4ccccc45)ccc32)cc1 ; c1ccc(-n2c3ccccc3c3c(N(c4ccc(-n5c6ccccc6c6c7ccccc7ccc65)cc4)c4ccc5c(c4)sc4ccccc45)cccc32)cc1 ; c1ccc(-n2c3ccccc3c3ccc(N(c4ccc(-c5ccc(-n6c7ccccc7c7c8ccccc8ccc76)cc5)cc4)c4ccc5sc6ccccc6c5c4)cc32)cc1]

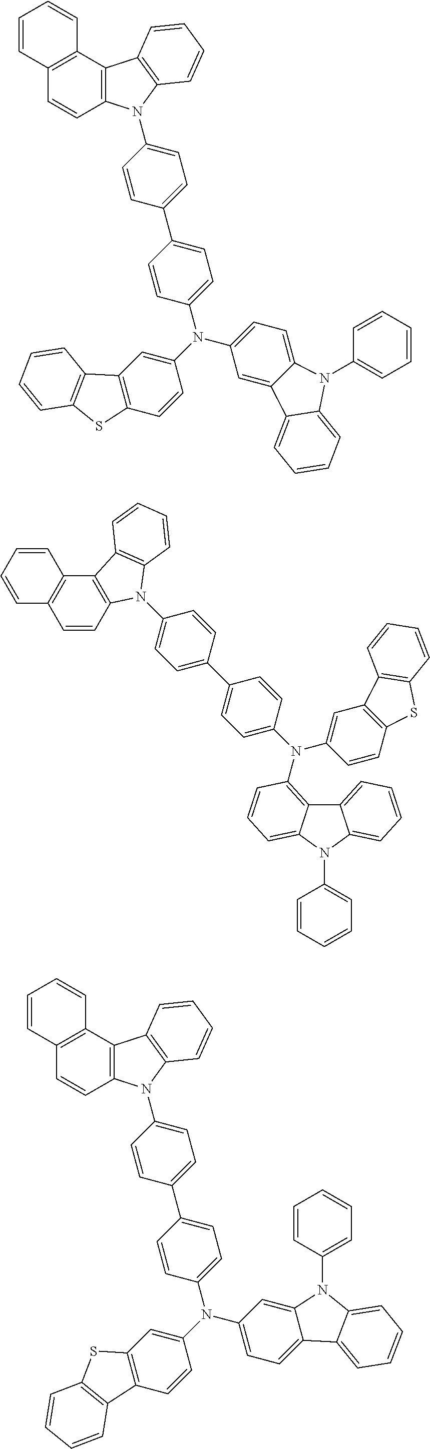 [SMILES: c1ccc(-n2c3ccccc3c3cc(N(c4ccc(-c5ccc(-n6c7ccccc7c7c8ccccc8ccc76)cc5)cc4)c4ccc5sc6ccccc6c5c4)ccc32)cc1 ; c1ccc(-n2c3ccccc3c3c(N(c4ccc(-c5ccc(-n6c7ccccc7c7c8ccccc8ccc76)cc5)cc4)c4ccc5sc6ccccc6c5c4)cccc32)cc1 ; c1ccc(-n2c3ccccc3c3ccc(N(c4ccc(-c5ccc(-n6c7ccccc7c7c8ccccc8ccc76)cc5)cc4)c4ccc5c(c4)sc4ccccc45)cc32)cc1]

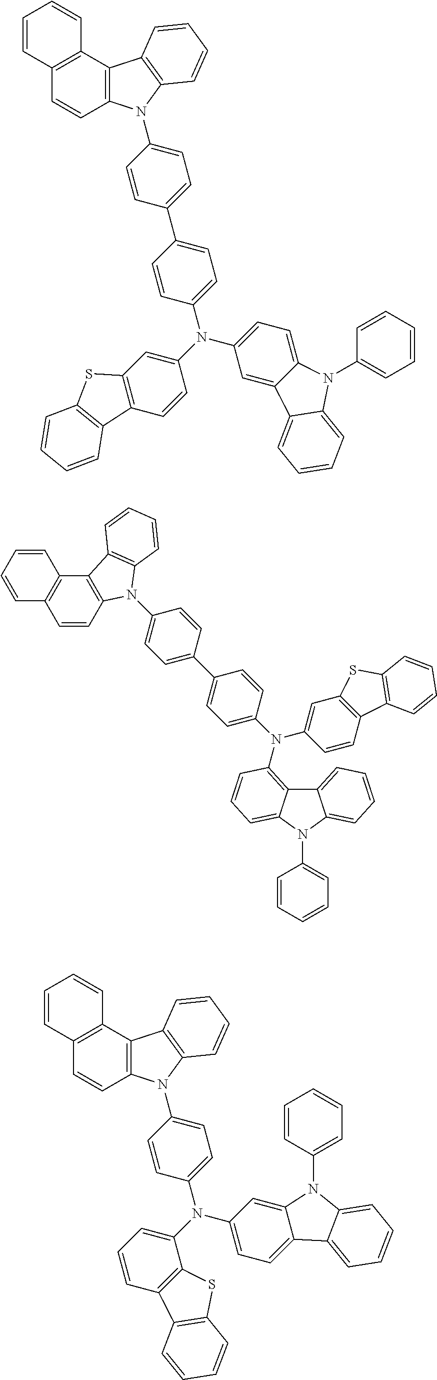 [SMILES: c1ccc(-n2c3ccccc3c3cc(N(c4ccc(-c5ccc(-n6c7ccccc7c7c8ccccc8ccc76)cc5)cc4)c4ccc5c(c4)sc4ccccc45)ccc32)cc1 ; c1ccc(-n2c3ccccc3c3c(N(c4ccc(-c5ccc(-n6c7ccccc7c7c8ccccc8ccc76)cc5)cc4)c4ccc5c(c4)sc4ccccc45)cccc32)cc1 ; c1ccc(-n2c3ccccc3c3ccc(N(c4ccc(-n5c6ccccc6c6c7ccccc7ccc65)cc4)c4cccc5c4sc4ccccc45)cc32)cc1]

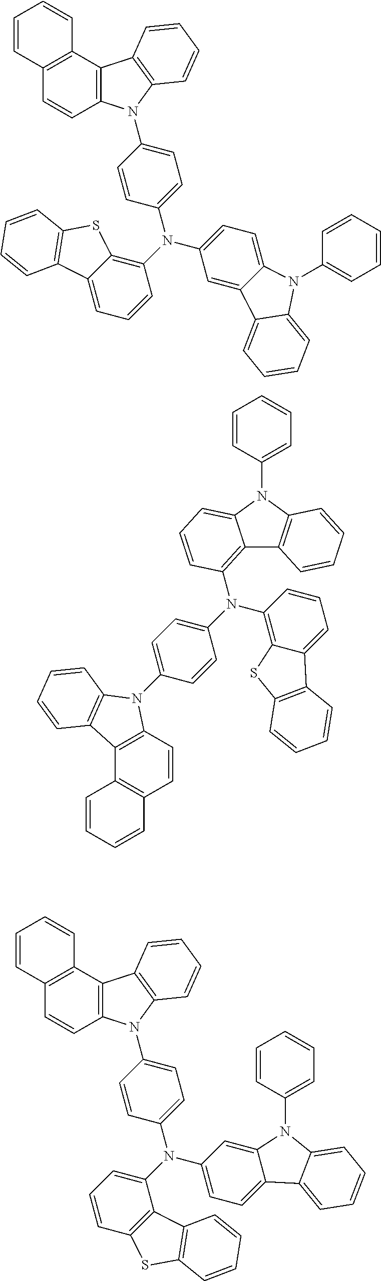

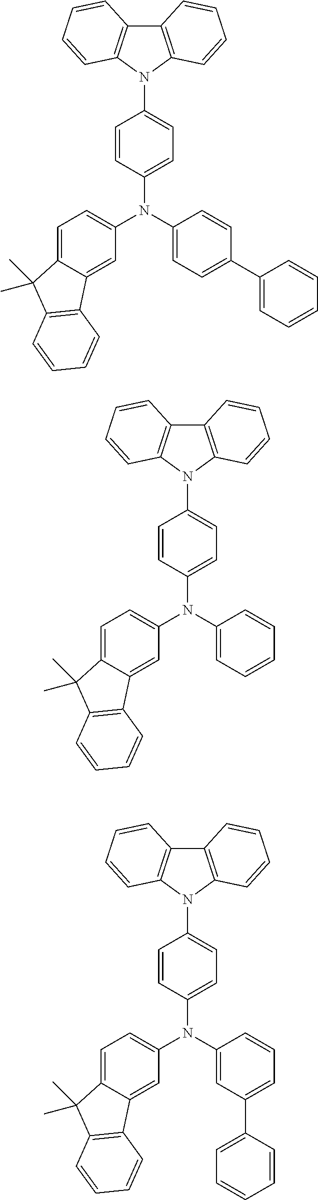 [SMILES: CC1(C)c2ccccc2-c2cc(N(c3ccc(-c4ccccc4)cc3)c3ccc(-n4c5ccccc5c5ccccc54)cc3)ccc21 ; CC1(C)c2ccccc2-c2cc(N(c3ccccc3)c3ccc(-n4c5ccccc5c5ccccc54)cc3)ccc21 ; CC1(C)c2ccccc2-c2cc(N(c3ccc(-n4c5ccccc5c5ccccc54)cc3)c3cccc(-c4ccccc4)c3)ccc21]

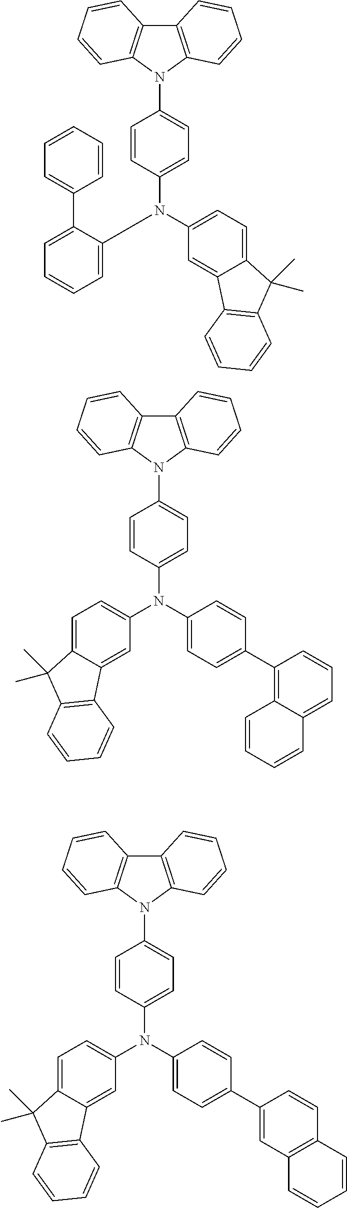

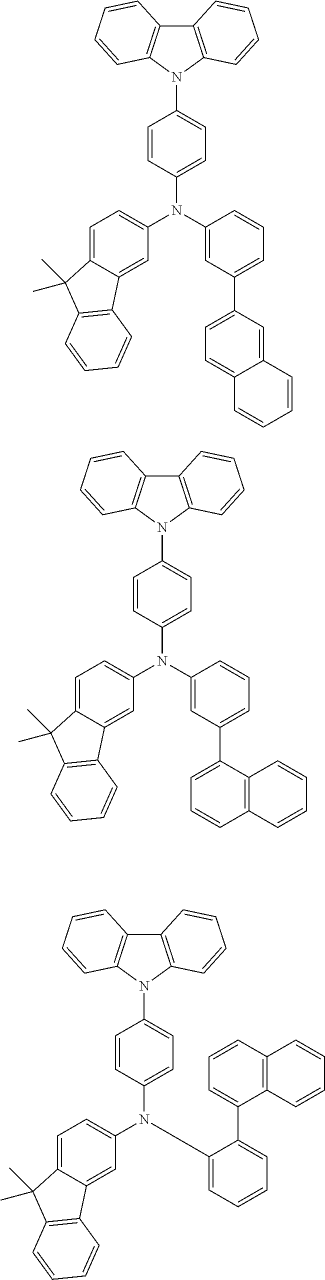

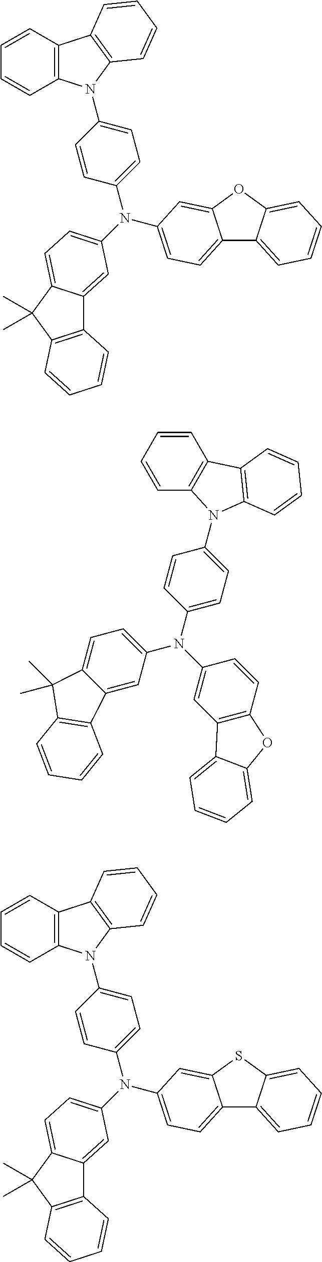 [SMILES: CC1(C)c2ccccc2-c2cc(N(c3ccc(-n4c5ccccc5c5ccccc54)cc3)c3ccc4c(c3)oc3ccccc34)ccc21 ; CC1(C)c2ccccc2-c2cc(N(c3ccc(-n4c5ccccc5c5ccccc54)cc3)c3ccc4oc5ccccc5c4c3)ccc21 ; CC1(C)c2ccccc2-c2cc(N(c3ccc(-n4c5ccccc5c5ccccc54)cc3)c3ccc4c(c3)sc3ccccc34)ccc21]

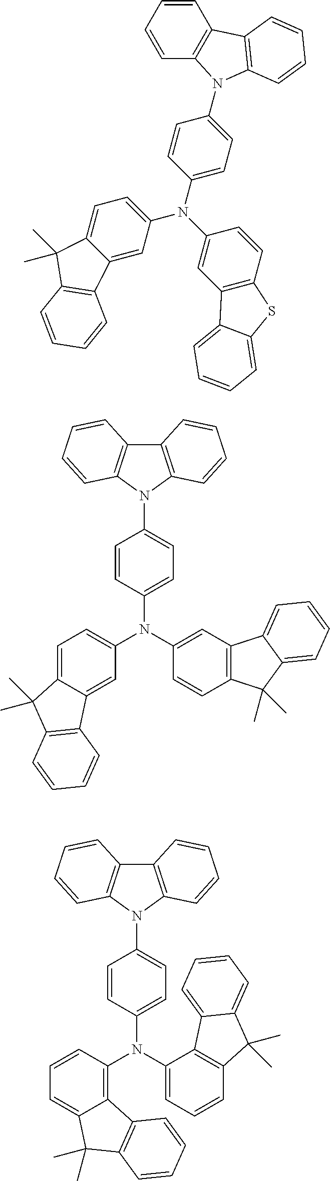

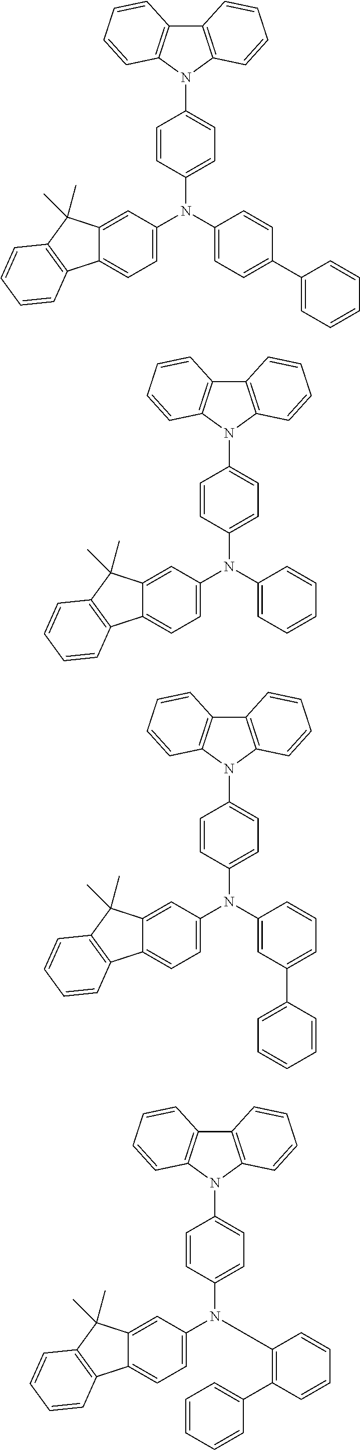

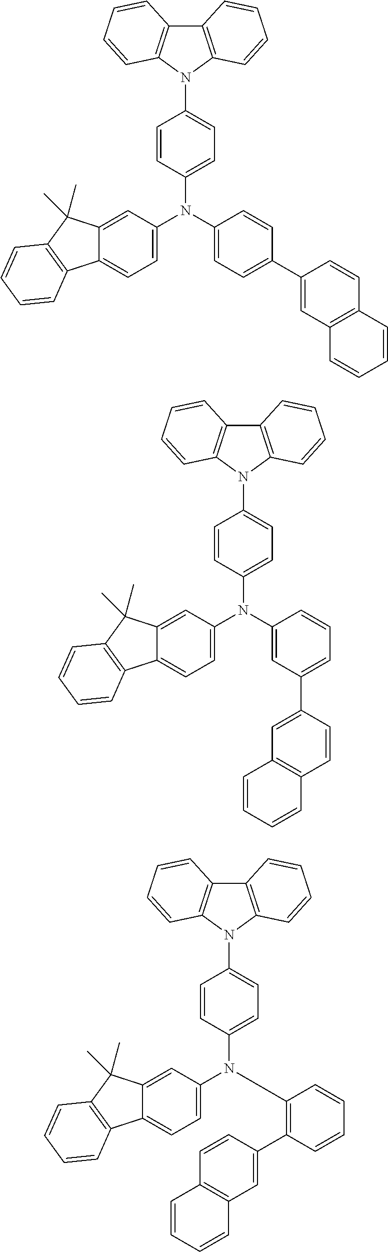

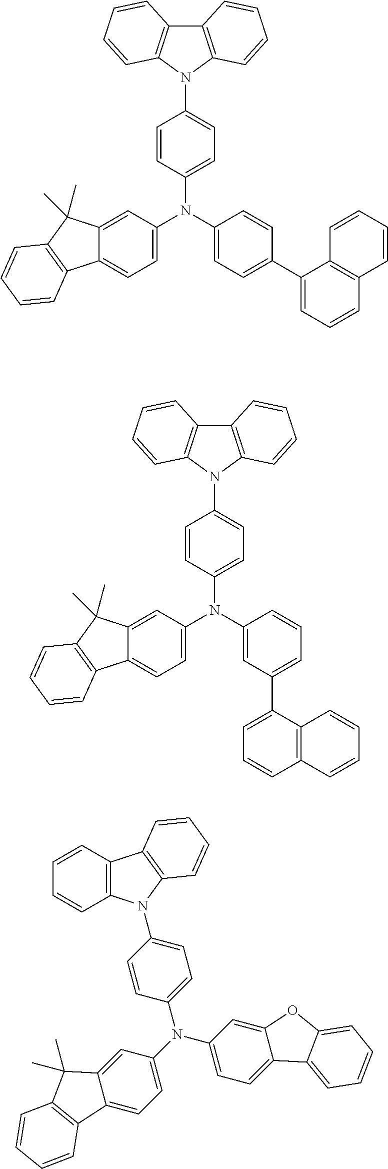 [SMILES: CC1(C)c2ccccc2-c2ccc(N(c3ccc(-c4cccc5ccccc45)cc3)c3ccc(-n4c5ccccc5c5ccccc54)cc3)cc21 ; CC1(C)c2ccccc2-c2ccc(N(c3ccc(-n4c5ccccc5c5ccccc54)cc3)c3cccc(-c4cccc5ccccc45)c3)cc21 ; CC1(C)c2ccccc2-c2ccc(N(c3ccc(-n4c5ccccc5c5ccccc54)cc3)c3ccc4c(c3)oc3ccccc34)cc21]

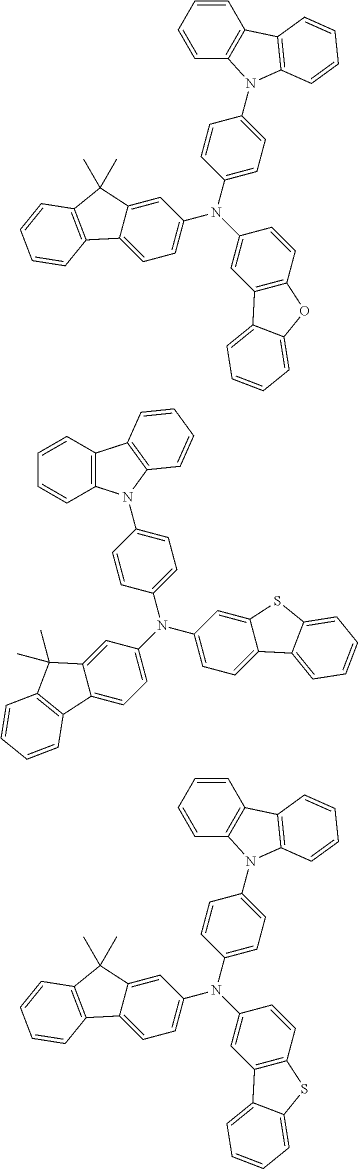 [SMILES: CC1(C)c2ccccc2-c2ccc(N(c3ccc(-n4c5ccccc5c5ccccc54)cc3)c3ccc4oc5ccccc5c4c3)cc21 ; CC1(C)c2ccccc2-c2ccc(N(c3ccc(-n4c5ccccc5c5ccccc54)cc3)c3ccc4c(c3)sc3ccccc34)cc21 ; CC1(C)c2ccccc2-c2ccc(N(c3ccc(-n4c5ccccc5c5ccccc54)cc3)c3ccc4sc5ccccc5c4c3)cc21]

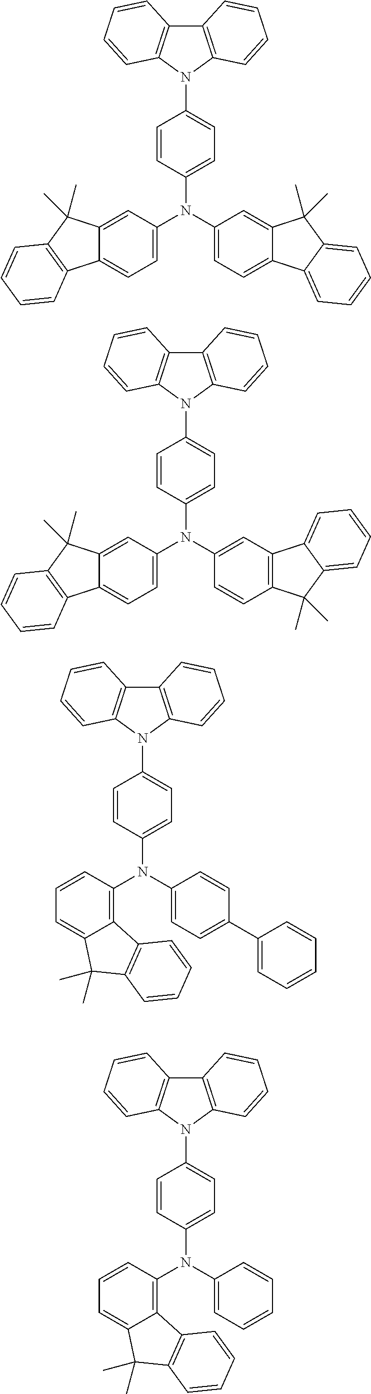

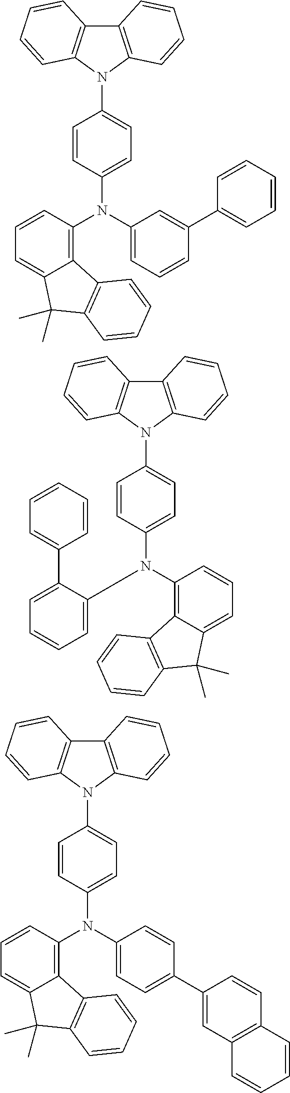

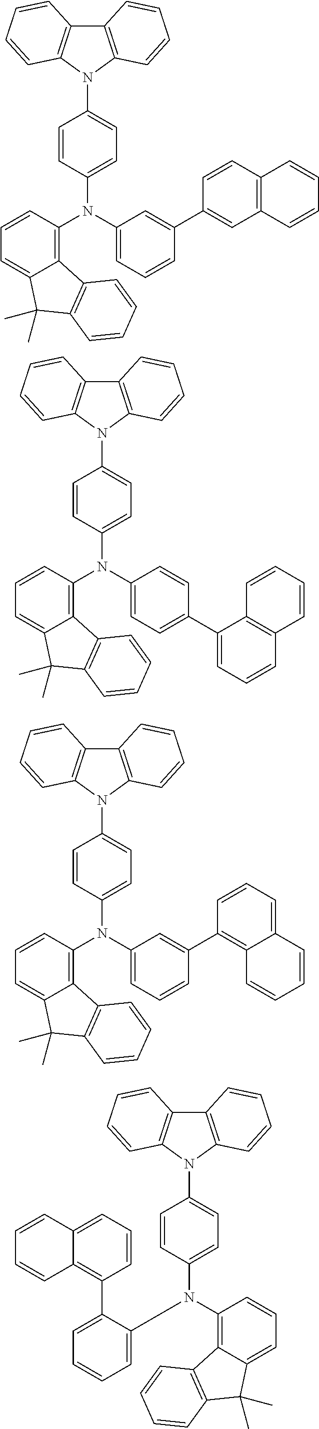

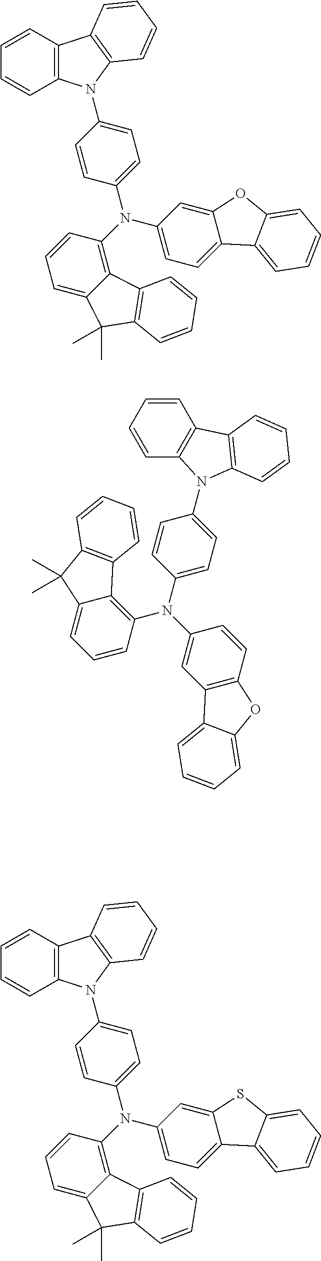 [SMILES: CC1(C)c2ccccc2-c2c(N(c3ccc(-n4c5ccccc5c5ccccc54)cc3)c3ccc4c(c3)oc3ccccc34)cccc21 ; CC1(C)c2ccccc2-c2c(N(c3ccc(-n4c5ccccc5c5ccccc54)cc3)c3ccc4oc5ccccc5c4c3)cccc21 ; CC1(C)c2ccccc2-c2c(N(c3ccc(-n4c5ccccc5c5ccccc54)cc3)c3ccc4c(c3)sc3ccccc34)cccc21]

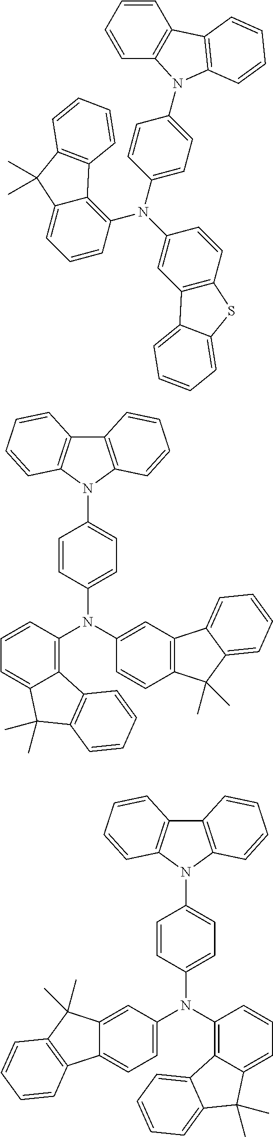

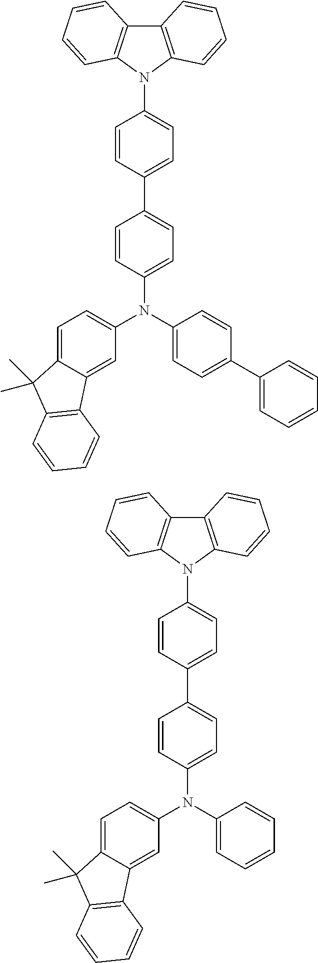

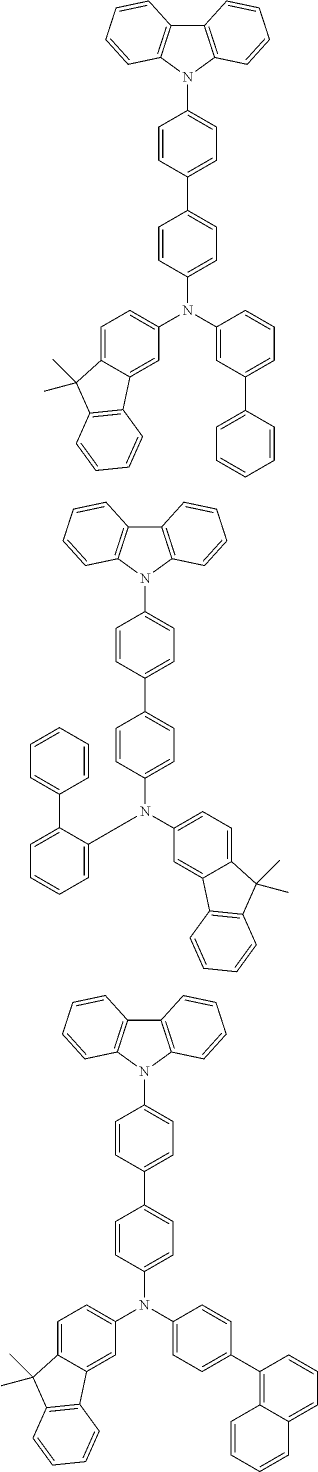

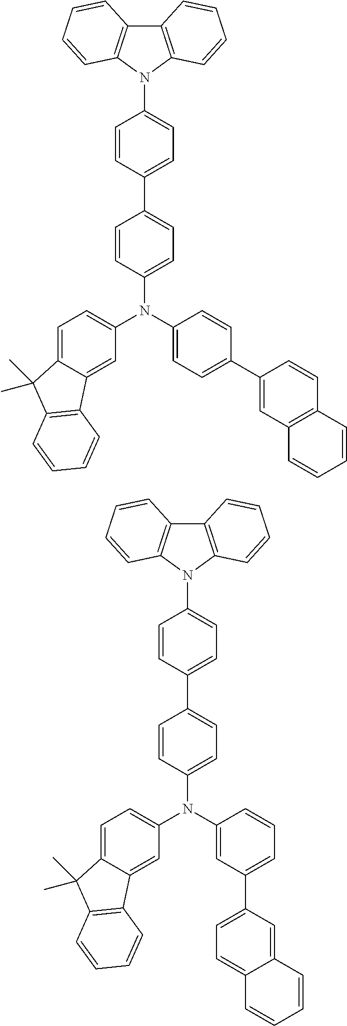 [SMILES: CC1(C)c2ccccc2-c2cc(N(c3ccc(-c4ccc(-n5c6ccccc6c6ccccc65)cc4)cc3)c3ccc(-c4ccc5ccccc5c4)cc3)ccc21 ; CC1(C)c2ccccc2-c2cc(N(c3ccc(-c4ccc(-n5c6ccccc6c6ccccc65)cc4)cc3)c3cccc(-c4ccc5ccccc5c4)c3)ccc21]

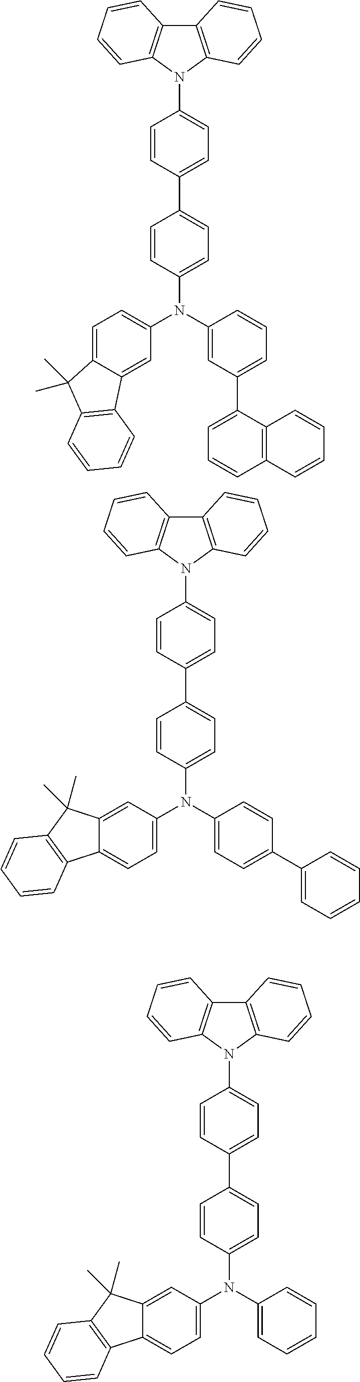

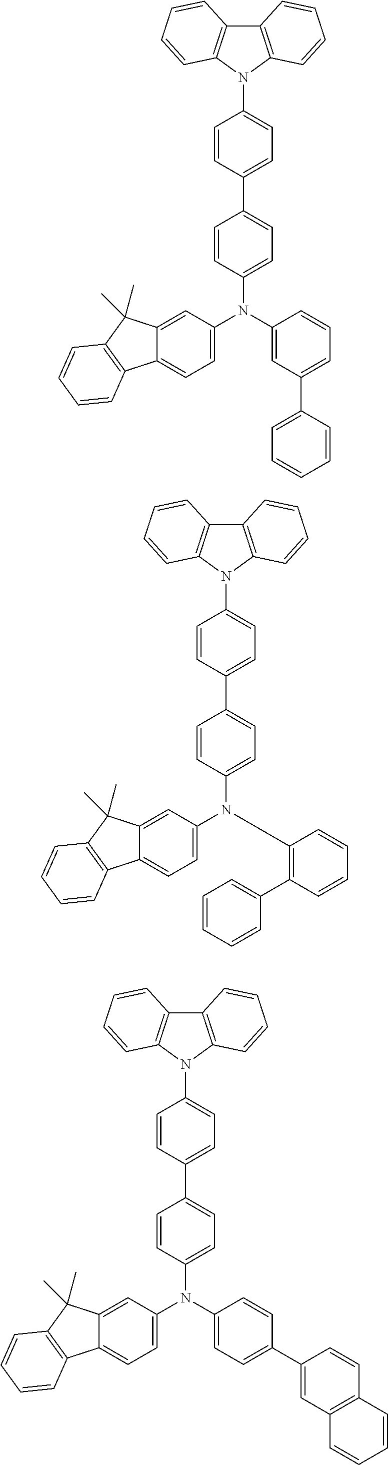

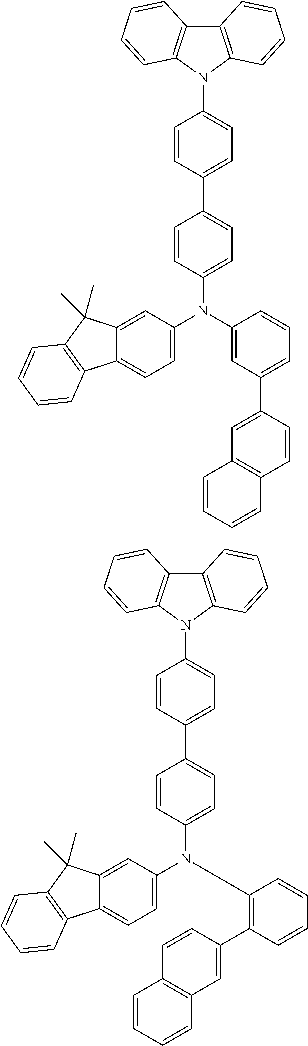

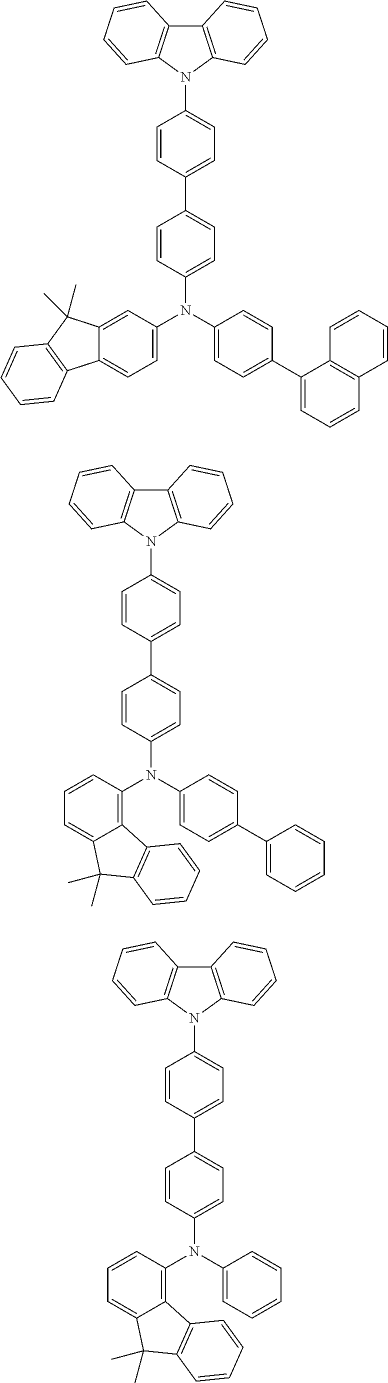

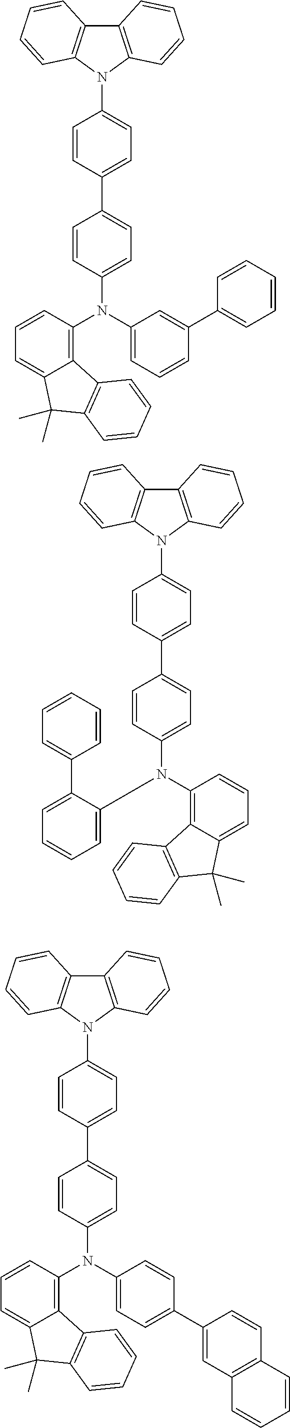

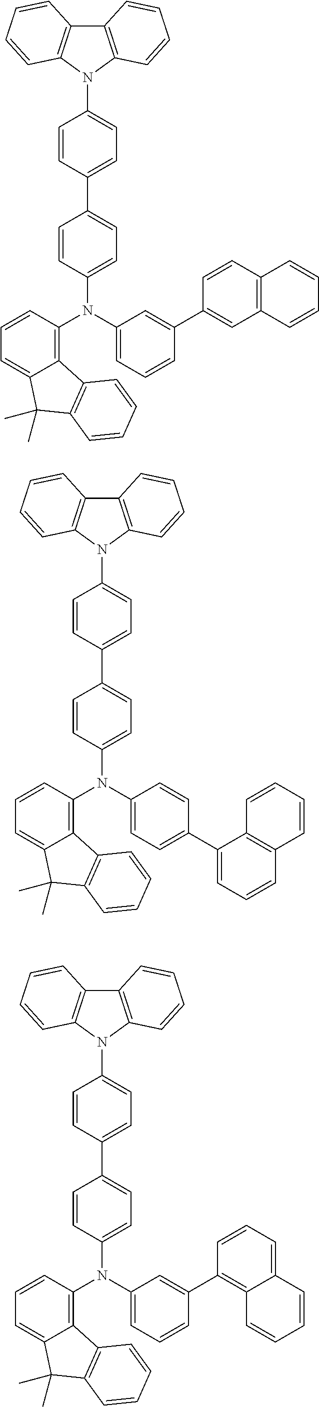

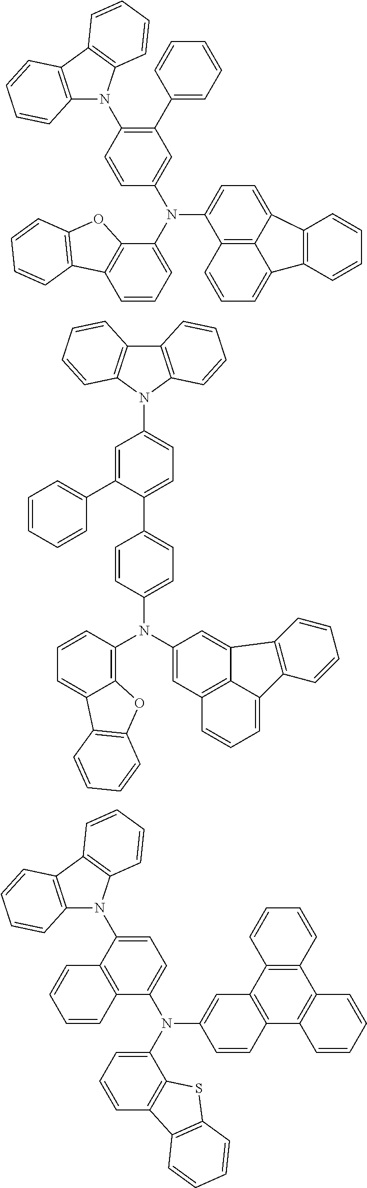

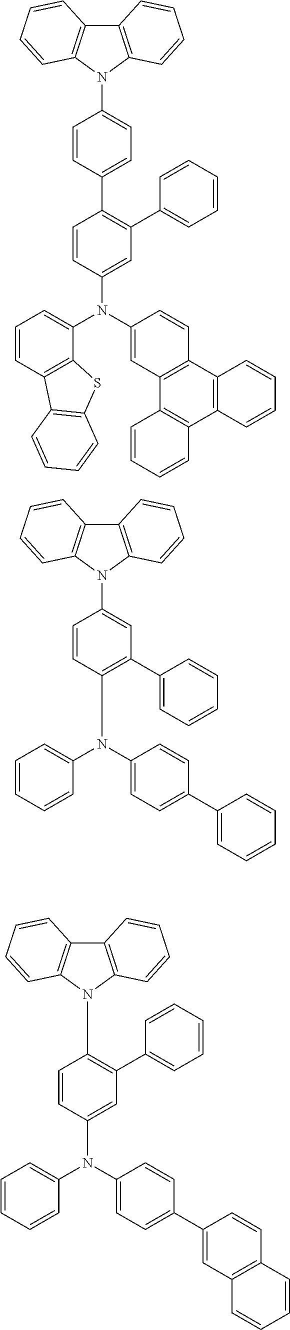

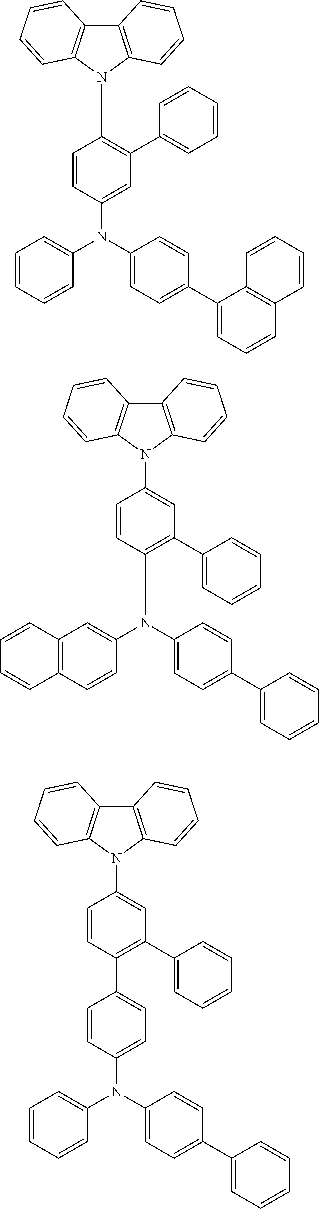

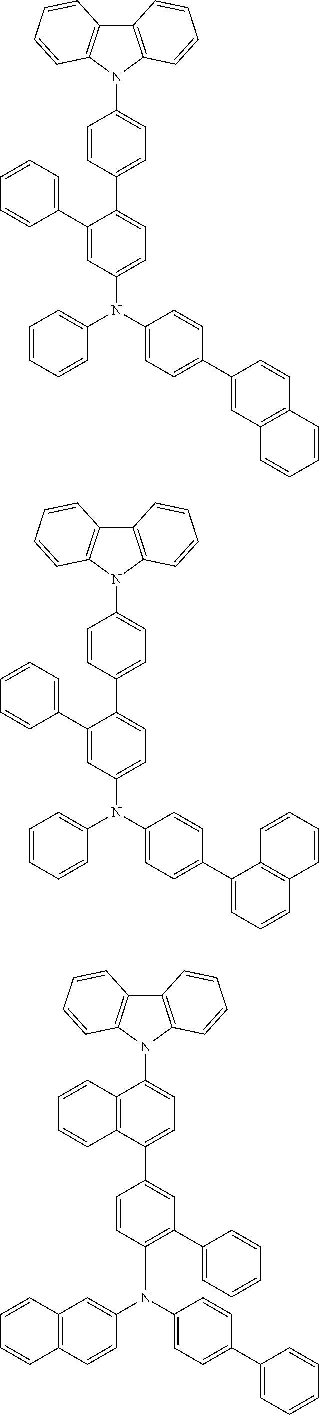 [SMILES: c1ccc(-c2cc(N(c3ccccc3)c3ccc(-c4ccc5ccccc5c4)cc3)ccc2-c2ccc(-n3c4ccccc4c4ccccc43)cc2)cc1 ; c1ccc(-c2cc(N(c3ccccc3)c3ccc(-c4cccc5ccccc45)cc3)ccc2-c2ccc(-n3c4ccccc4c4ccccc43)cc2)cc1 ; c1ccc(-c2ccc(N(c3ccc4ccccc4c3)c3ccc(-c4ccc(-n5c6ccccc6c6ccccc65)c5ccccc45)cc3-c3ccccc3)cc2)cc1]

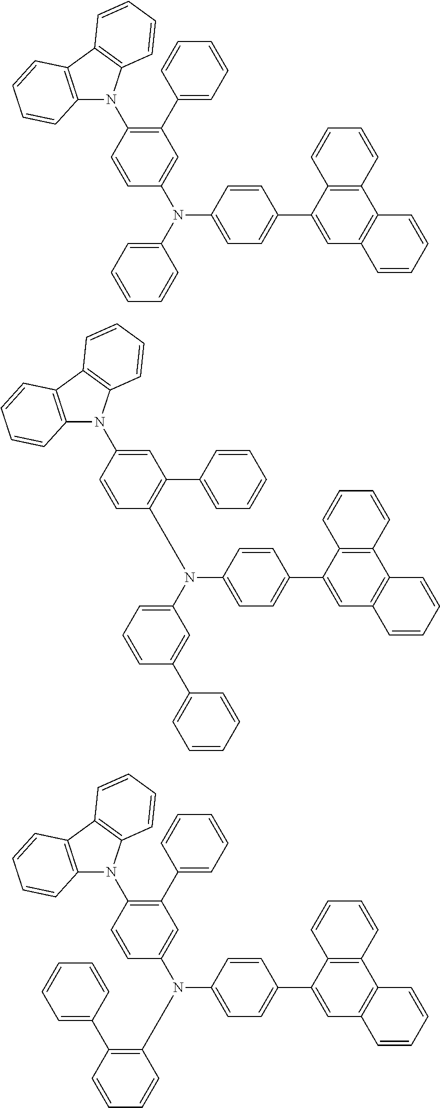

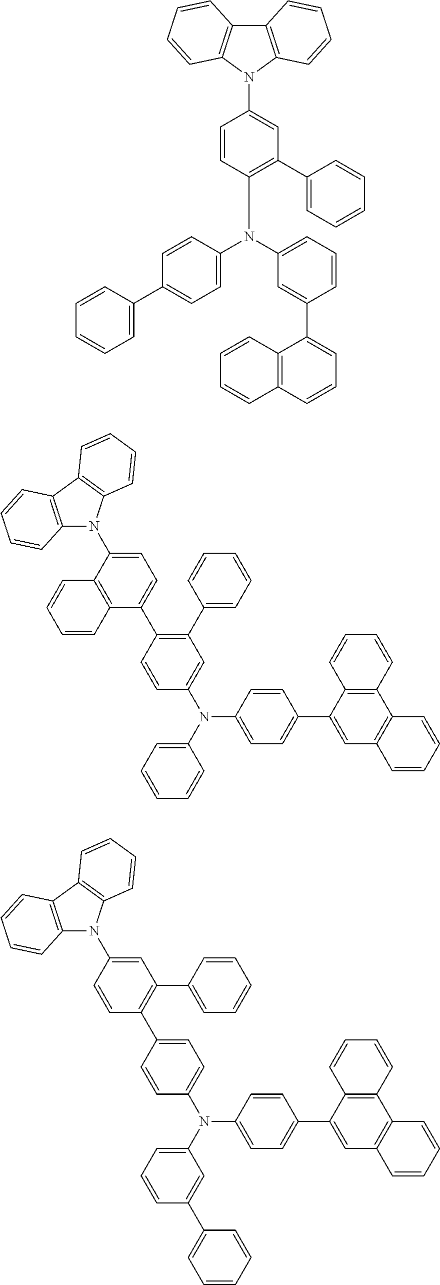 [SMILES: c1ccc(-c2ccc(N(c3cccc(-c4cccc5ccccc45)c3)c3ccc(-n4c5ccccc5c5ccccc54)cc3-c3ccccc3)cc2)cc1 ; c1ccc(-c2cc(N(c3ccccc3)c3ccc(-c4cc5ccccc5c5ccccc45)cc3)ccc2-c2ccc(-n3c4ccccc4c4ccccc43)c3ccccc23)cc1 ; c1ccc(-c2cccc(N(c3ccc(-c4ccc(-n5c6ccccc6c6ccccc65)cc4-c4ccccc4)cc3)c3ccc(-c4cc5ccccc5c5ccccc45)cc3)c2)cc1]

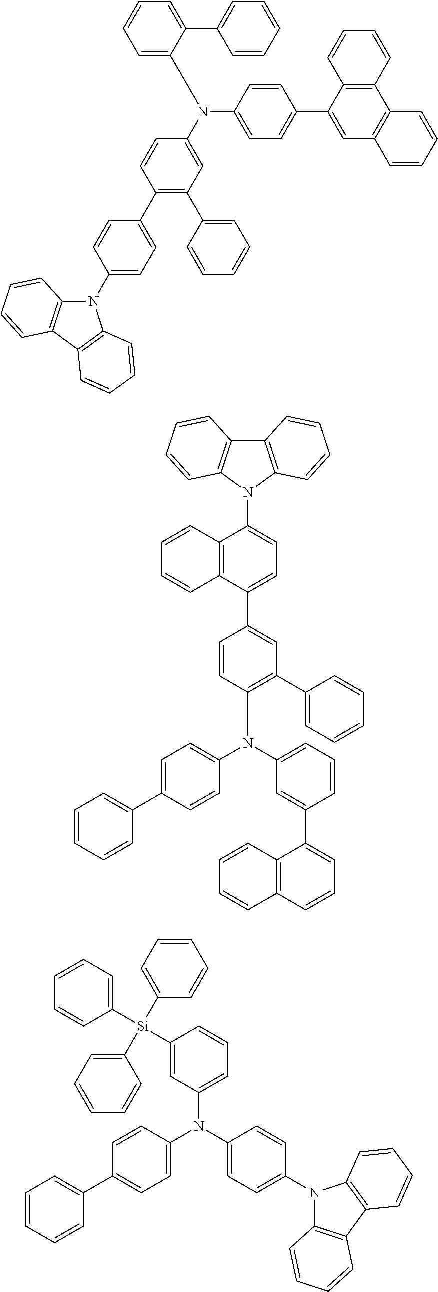 [SMILES: c1ccc(-c2cc(N(c3ccc(-c4cc5ccccc5c5ccccc45)cc3)c3ccccc3-c3ccccc3)ccc2-c2ccc(-n3c4ccccc4c4ccccc43)cc2)cc1 ; c1ccc(-c2ccc(N(c3cccc(-c4cccc5ccccc45)c3)c3ccc(-c4ccc(-n5c6ccccc6c6ccccc65)c5ccccc45)cc3-c3ccccc3)cc2)cc1 ; c1ccc(-c2ccc(N(c3ccc(-n4c5ccccc5c5ccccc54)cc3)c3cccc([Si](c4ccccc4)(c4ccccc4)c4ccccc4)c3)cc2)cc1]

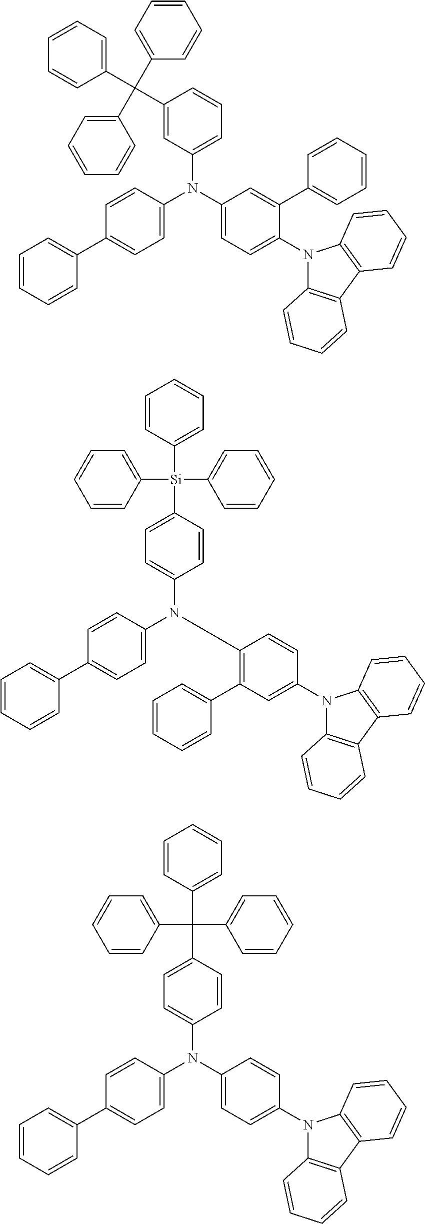 [SMILES: c1ccc(-c2ccc(N(c3cccc(C(c4ccccc4)(c4ccccc4)c4ccccc4)c3)c3ccc(-n4c5ccccc5c5ccccc54)c(-c4ccccc4)c3)cc2)cc1 ; c1ccc(-c2ccc(N(c3ccc([Si](c4ccccc4)(c4ccccc4)c4ccccc4)cc3)c3ccc(-n4c5ccccc5c5ccccc54)cc3-c3ccccc3)cc2)cc1 ; c1ccc(-c2ccc(N(c3ccc(-n4c5ccccc5c5ccccc54)cc3)c3ccc(C(c4ccccc4)(c4ccccc4)c4ccccc4)cc3)cc2)cc1]

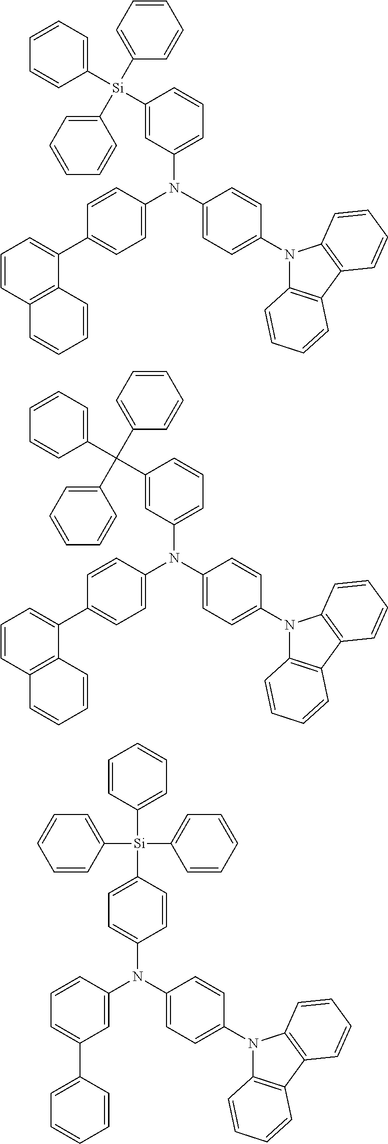

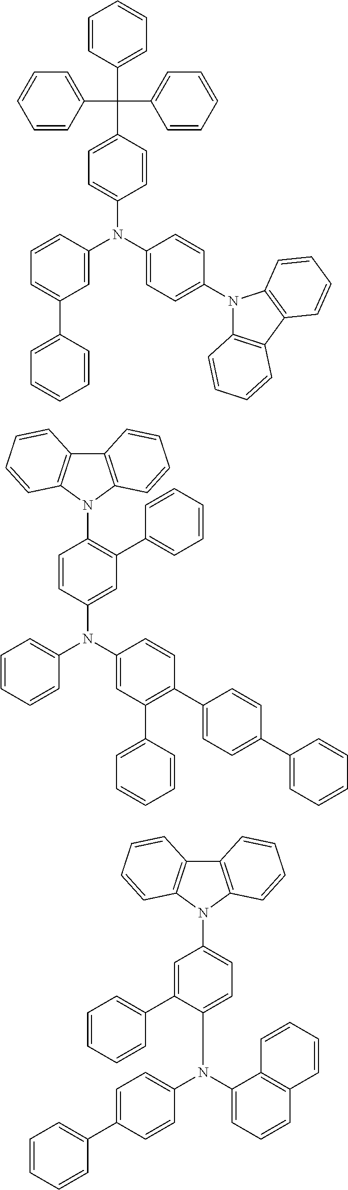

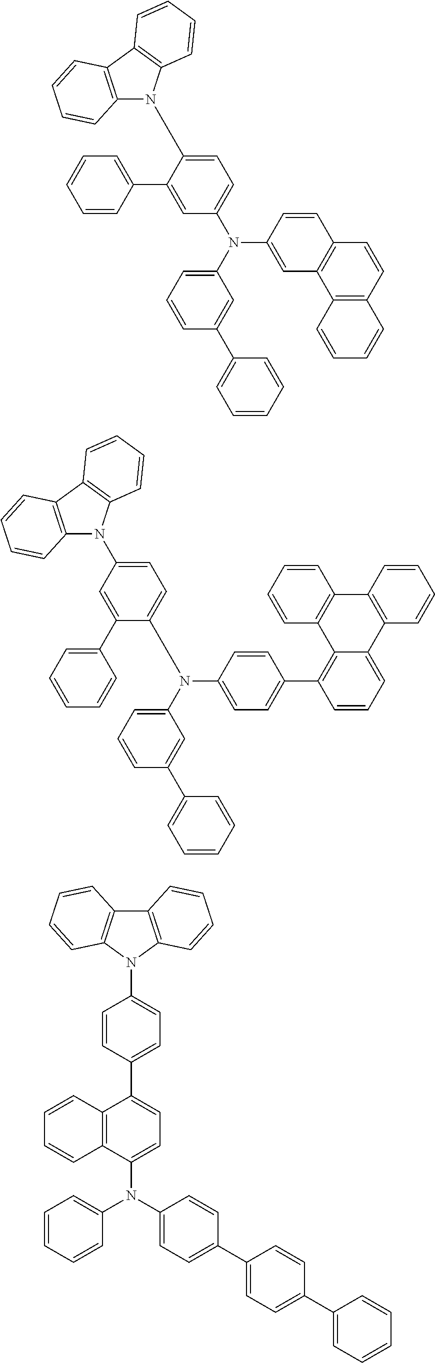

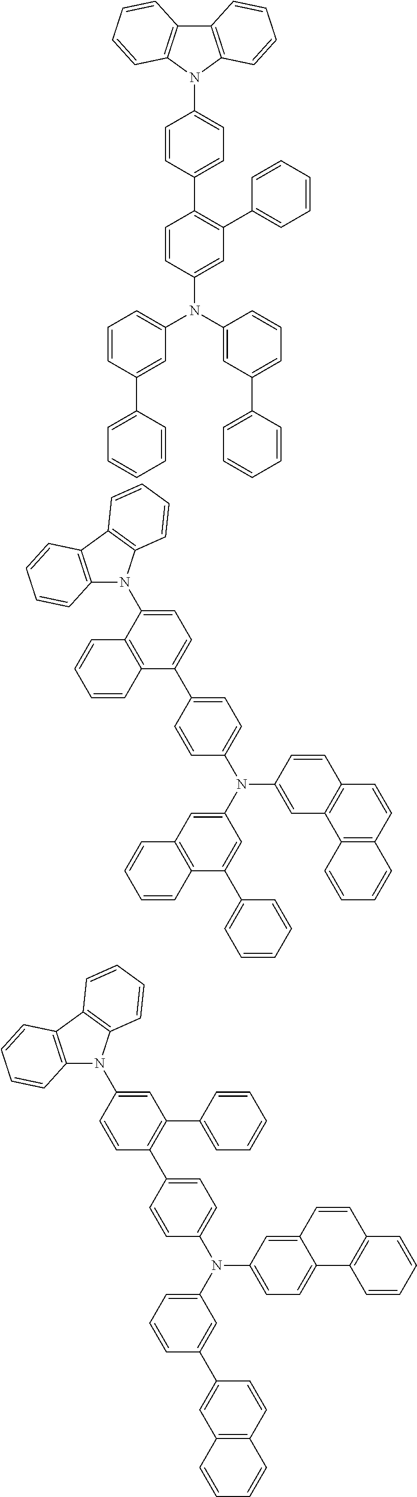

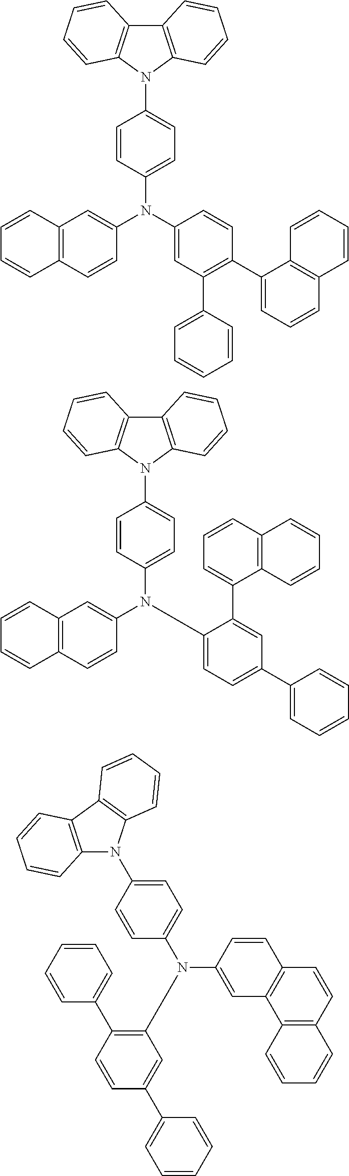

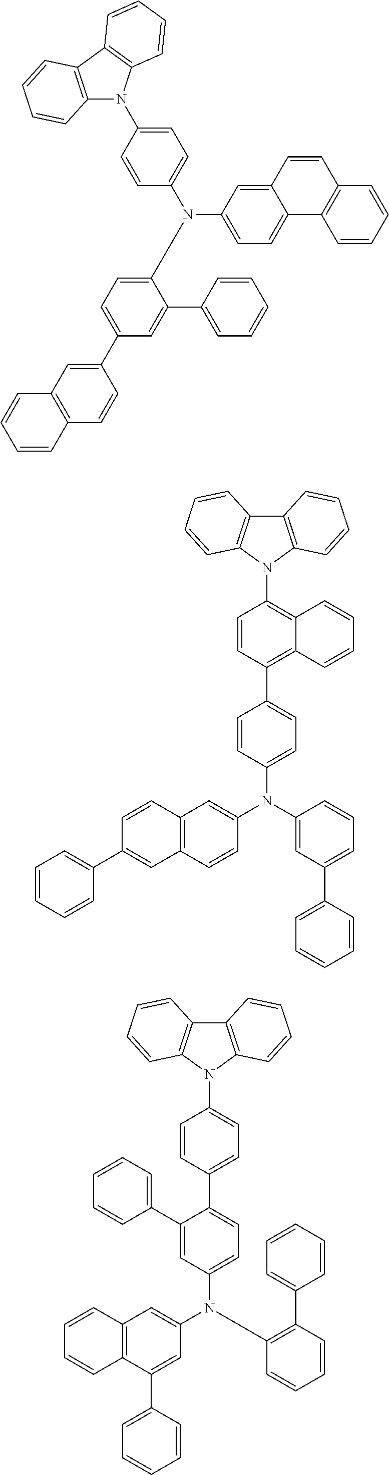 [SMILES: c1ccc(-c2cc(-c3ccc4ccccc4c3)ccc2N(c2ccc(-n3c4ccccc4c4ccccc43)cc2)c2ccc3c(ccc4ccccc43)c2)cc1 ; c1ccc(-c2cccc(N(c3ccc(-c4ccc(-n5c6ccccc6c6ccccc65)c5ccccc45)cc3)c3ccc4cc(-c5ccccc5)ccc4c3)c2)cc1 ; c1ccc(-c2cc(N(c3cc(-c4ccccc4)c4ccccc4c3)c3ccccc3-c3ccccc3)ccc2-c2ccc(-n3c4ccccc4c4ccccc43)cc2)cc1]

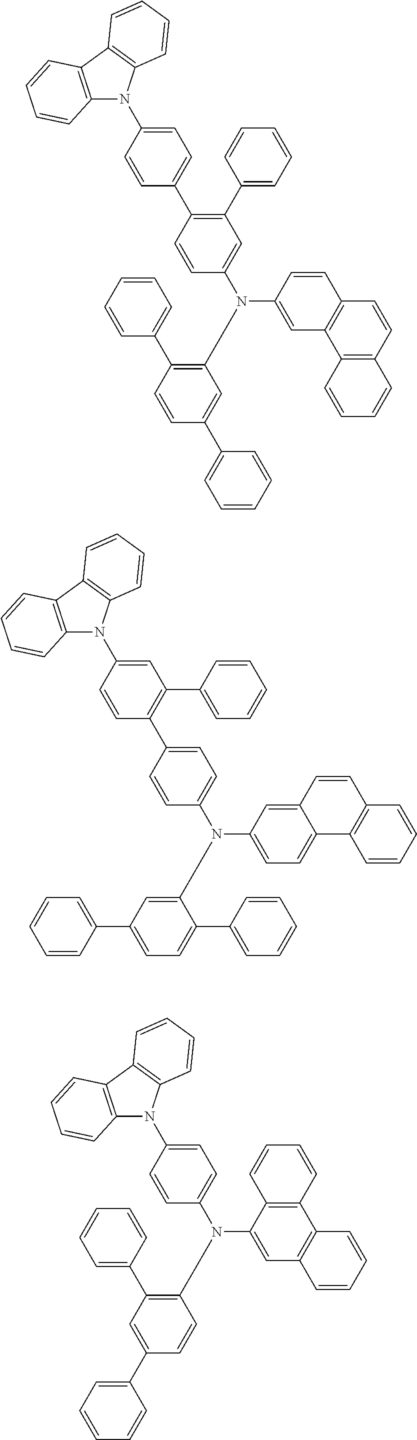 [SMILES: c1ccc(-c2ccc(-c3ccccc3)c(N(c3ccc(-c4ccc(-n5c6ccccc6c6ccccc65)cc4)c(-c4ccccc4)c3)c3ccc4ccc5ccccc5c4c3)c2)cc1 ; c1ccc(-c2ccc(-c3ccccc3)c(N(c3ccc(-c4ccc(-n5c6ccccc6c6ccccc65)cc4-c4ccccc4)cc3)c3ccc4c(ccc5ccccc54)c3)c2)cc1 ; c1ccc(-c2ccc(N(c3ccc(-n4c5ccccc5c5ccccc54)cc3)c3cc4ccccc4c4ccccc34)c(-c3ccccc3)c2)cc1]

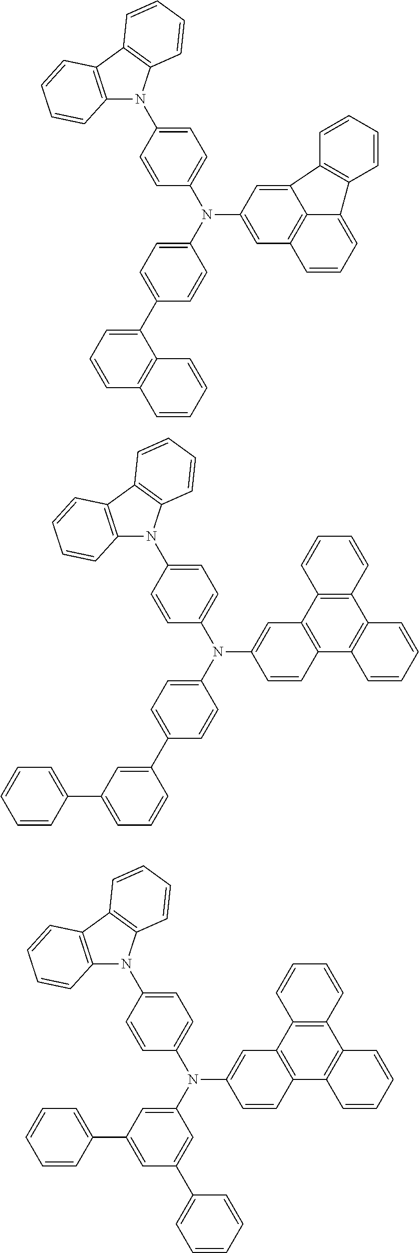

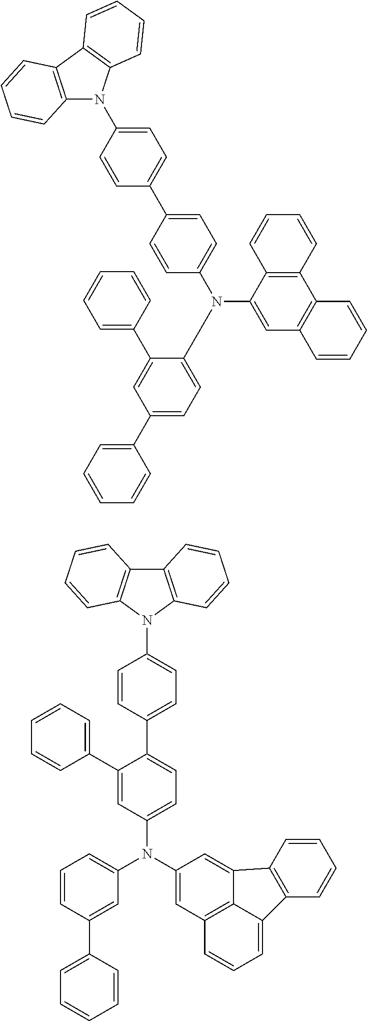

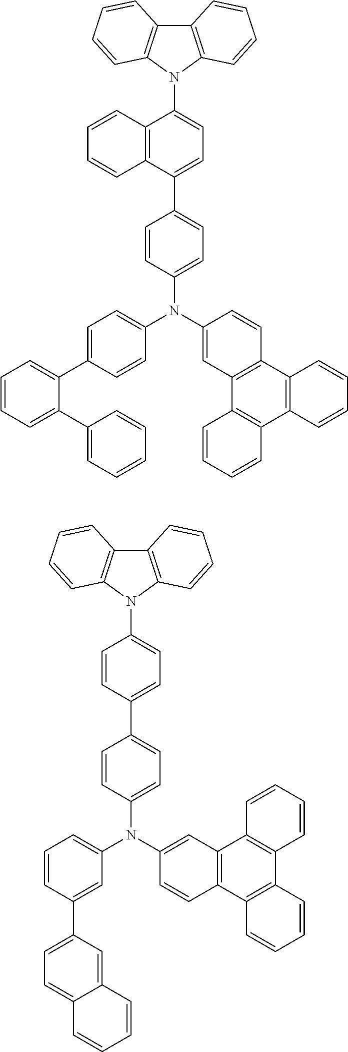 [SMILES: c1ccc(-c2ccccc2-c2ccc(N(c3ccc(-c4ccc(-n5c6ccccc6c6ccccc65)c5ccccc45)cc3)c3ccc4c5ccccc5c5ccccc5c4c3)cc2)cc1 ; c1cc(-c2ccc3ccccc3c2)cc(N(c2ccc(-c3ccc(-n4c5ccccc5c5ccccc54)cc3)cc2)c2ccc3c4ccccc4c4ccccc4c3c2)c1]

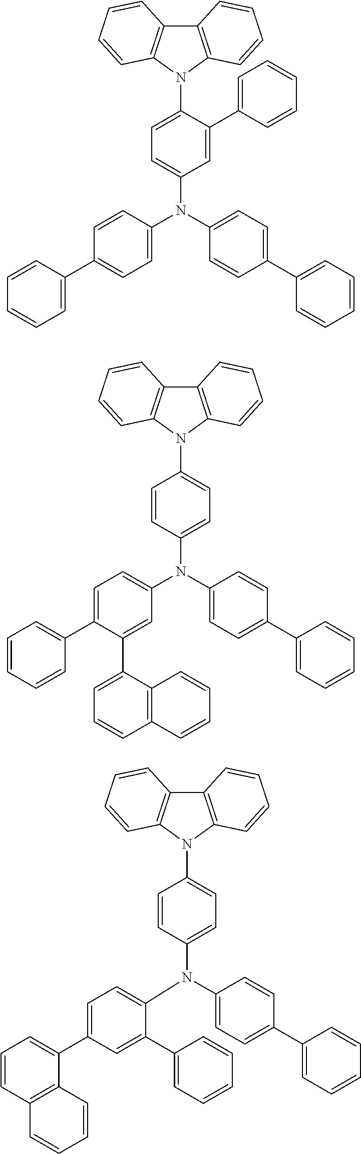

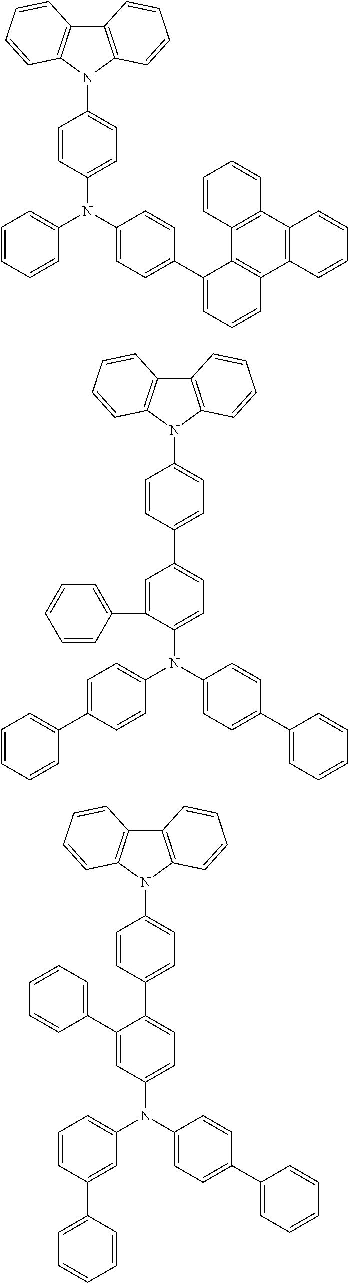

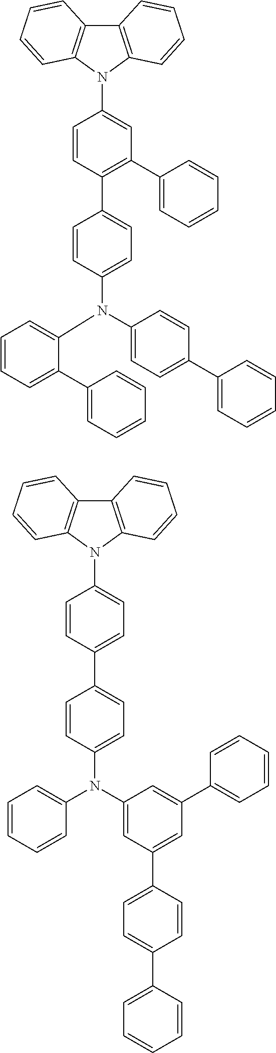

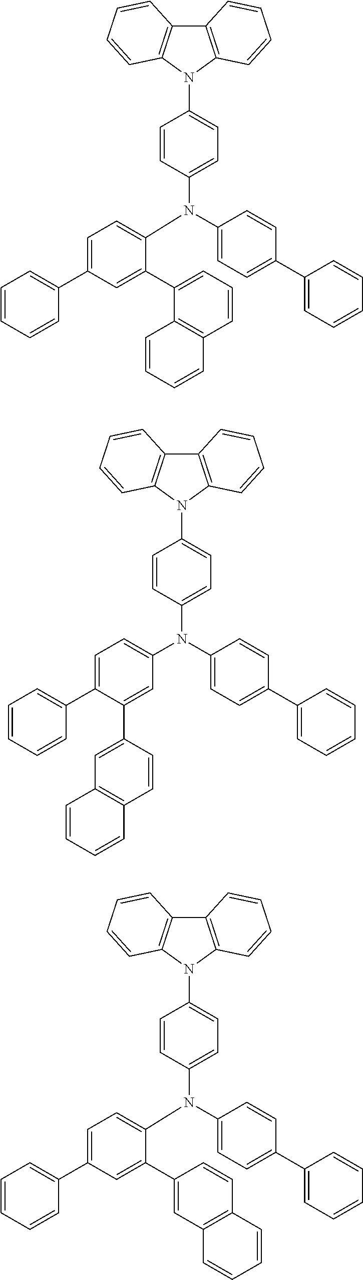 [SMILES: c1ccc(-c2ccc(N(c3ccc(-n4c5ccccc5c5ccccc54)cc3)c3ccc(-c4ccccc4)cc3-c3cccc4ccccc34)cc2)cc1 ; c1ccc(-c2ccc(N(c3ccc(-n4c5ccccc5c5ccccc54)cc3)c3ccc(-c4ccccc4)c(-c4ccc5ccccc5c4)c3)cc2)cc1 ; c1ccc(-c2ccc(N(c3ccc(-n4c5ccccc5c5ccccc54)cc3)c3ccc(-c4ccccc4)cc3-c3ccc4ccccc4c3)cc2)cc1]

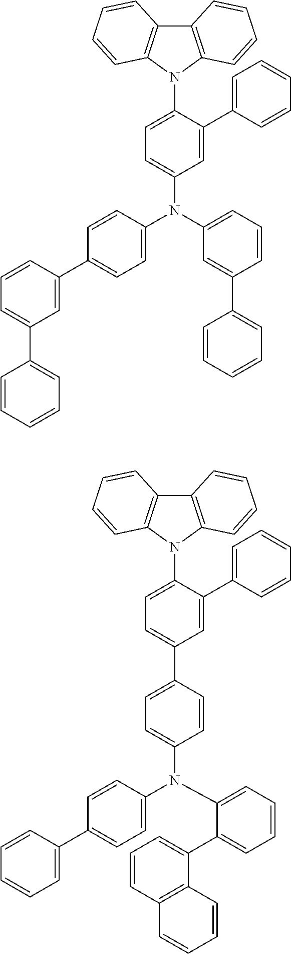 [SMILES: c1ccc(-c2cccc(-c3ccc(N(c4cccc(-c5ccccc5)c4)c4ccc(-n5c6ccccc6c6ccccc65)c(-c5ccccc5)c4)cc3)c2)cc1 ; c1ccc(-c2ccc(N(c3ccc(-c4ccc(-n5c6ccccc6c6ccccc65)c(-c5ccccc5)c4)cc3)c3ccccc3-c3cccc4ccccc34)cc2)cc1]

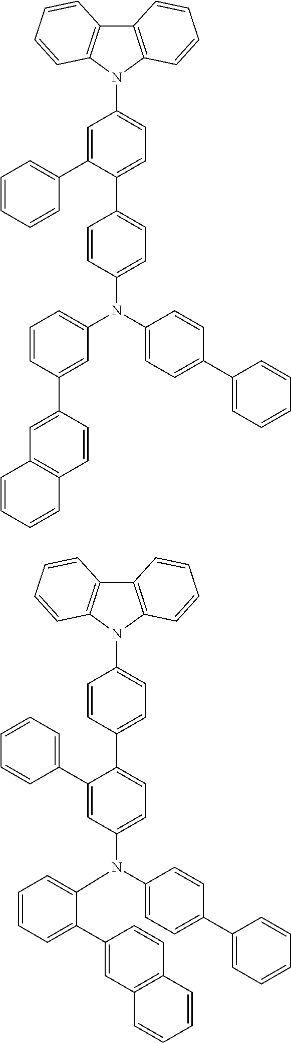

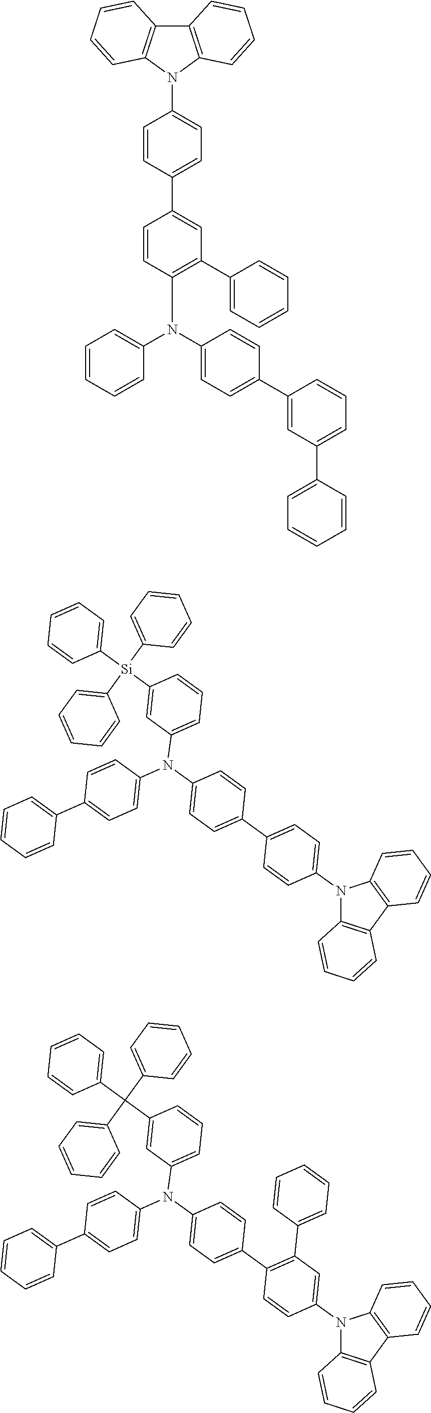 [SMILES: c1ccc(-c2cccc(-c3ccc(N(c4ccccc4)c4ccc(-c5ccc(-n6c7ccccc7c7ccccc76)cc5)cc4-c4ccccc4)cc3)c2)cc1 ; c1ccc(-c2ccc(N(c3ccc(-c4ccc(-n5c6ccccc6c6ccccc65)cc4)cc3)c3cccc([Si](c4ccccc4)(c4ccccc4)c4ccccc4)c3)cc2)cc1 ; c1ccc(-c2ccc(N(c3ccc(-c4ccc(-n5c6ccccc6c6ccccc65)cc4-c4ccccc4)cc3)c3cccc(C(c4ccccc4)(c4ccccc4)c4ccccc4)c3)cc2)cc1]

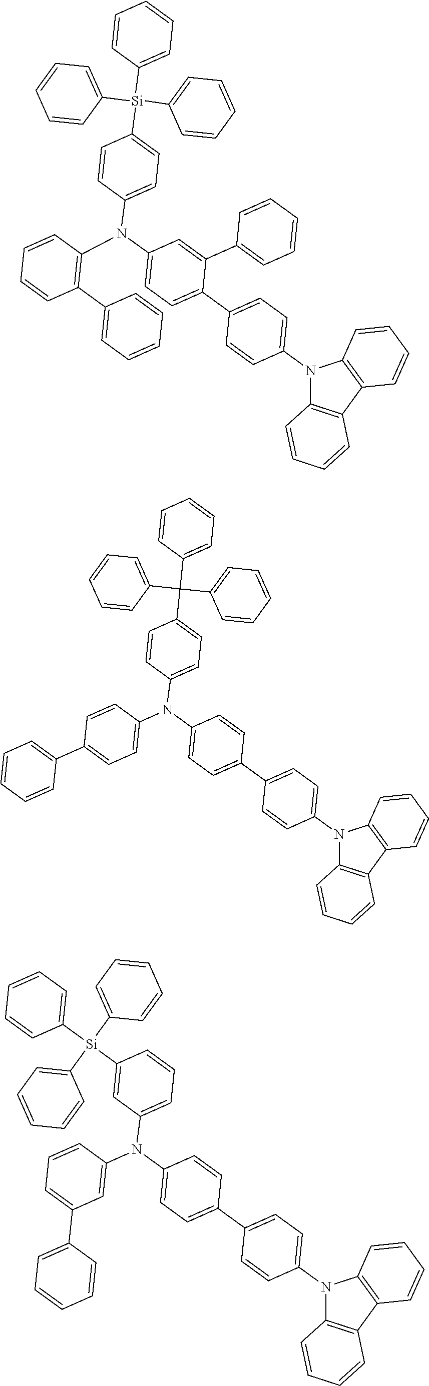 [SMILES: c1ccc(-c2cc(N(c3ccc([Si](c4ccccc4)(c4ccccc4)c4ccccc4)cc3)c3ccccc3-c3ccccc3)ccc2-c2ccc(-n3c4ccccc4c4ccccc43)cc2)cc1 ; c1ccc(-c2ccc(N(c3ccc(-c4ccc(-n5c6ccccc6c6ccccc65)cc4)cc3)c3ccc(C(c4ccccc4)(c4ccccc4)c4ccccc4)cc3)cc2)cc1 ; c1ccc(-c2cccc(N(c3ccc(-c4ccc(-n5c6ccccc6c6ccccc65)cc4)cc3)c3cccc([Si](c4ccccc4)(c4ccccc4)c4ccccc4)c3)c2)cc1]

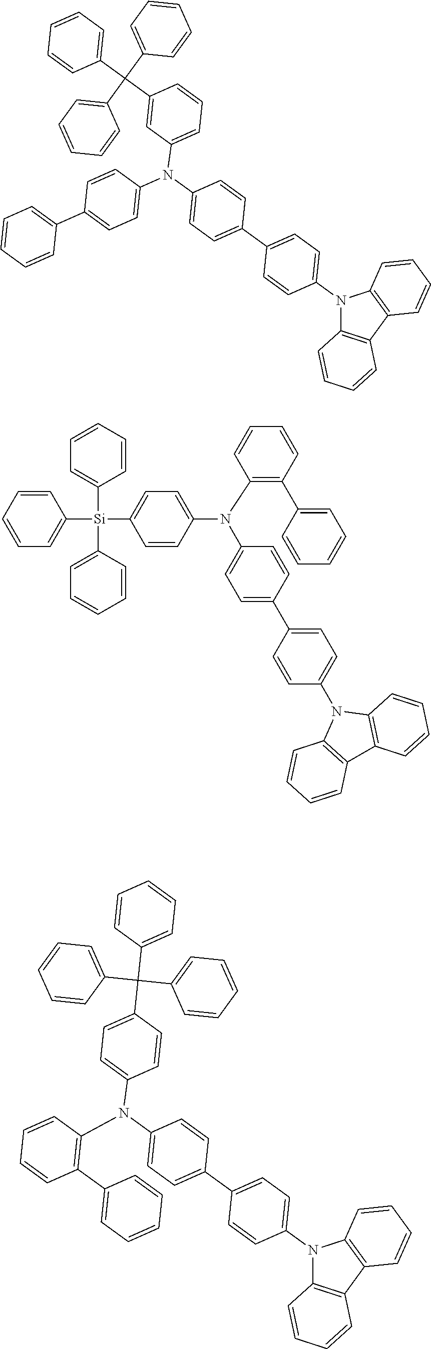 [SMILES: c1ccc(-c2ccc(N(c3ccc(-c4ccc(-n5c6ccccc6c6ccccc65)cc4)cc3)c3cccc(C(c4ccccc4)(c4ccccc4)c4ccccc4)c3)cc2)cc1 ; c1ccc(-c2ccccc2N(c2ccc(-c3ccc(-n4c5ccccc5c5ccccc54)cc3)cc2)c2ccc([Si](c3ccccc3)(c3ccccc3)c3ccccc3)cc2)cc1 ; c1ccc(-c2ccccc2N(c2ccc(-c3ccc(-n4c5ccccc5c5ccccc54)cc3)cc2)c2ccc(C(c3ccccc3)(c3ccccc3)c3ccccc3)cc2)cc1]

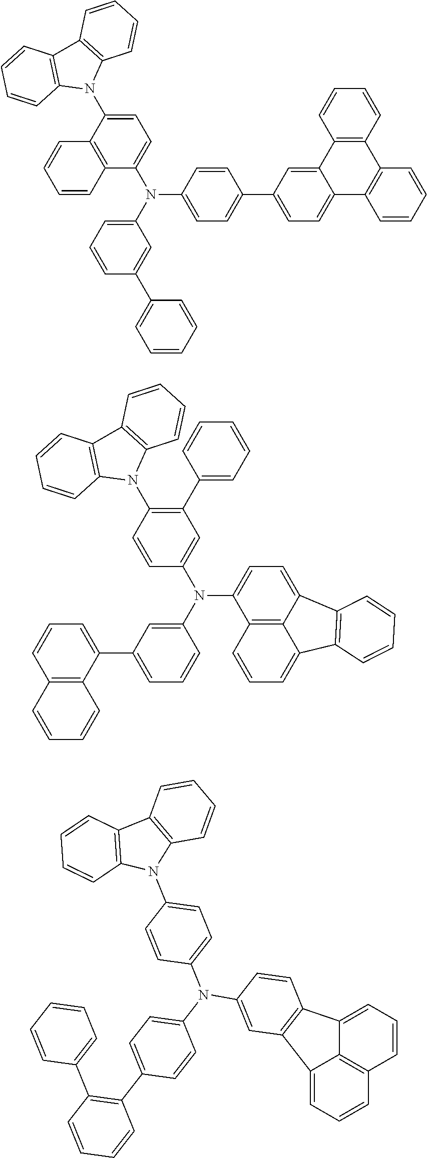

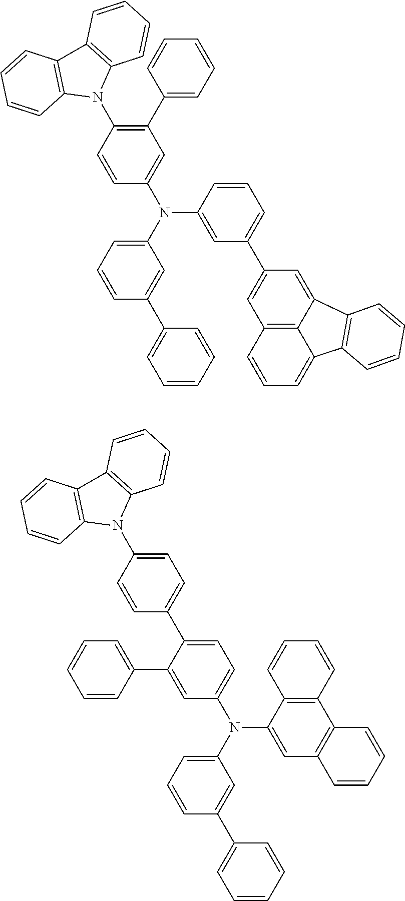 [SMILES: c1ccc(-c2cccc(N(c3cccc(-c4cc5c6c(cccc6c4)-c4ccccc4-5)c3)c3ccc(-n4c5ccccc5c5ccccc54)c(-c4ccccc4)c3)c2)cc1 ; c1ccc(-c2cccc(N(c3ccc(-c4ccc(-n5c6ccccc6c6ccccc65)cc4)c(-c4ccccc4)c3)c3cc4ccccc4c4ccccc34)c2)cc1]

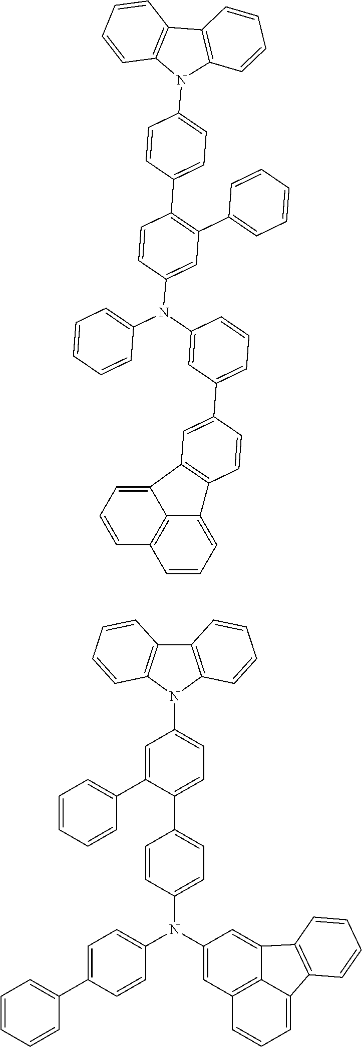 [SMILES: c1ccc(-c2cc(N(c3ccccc3)c3cccc(-c4ccc5c(c4)-c4cccc6cccc-5c46)c3)ccc2-c2ccc(-n3c4ccccc4c4ccccc43)cc2)cc1 ; c1ccc(-c2ccc(N(c3ccc(-c4ccc(-n5c6ccccc6c6ccccc65)cc4-c4ccccc4)cc3)c3cc4c5c(cccc5c3)-c3ccccc3-4)cc2)cc1]

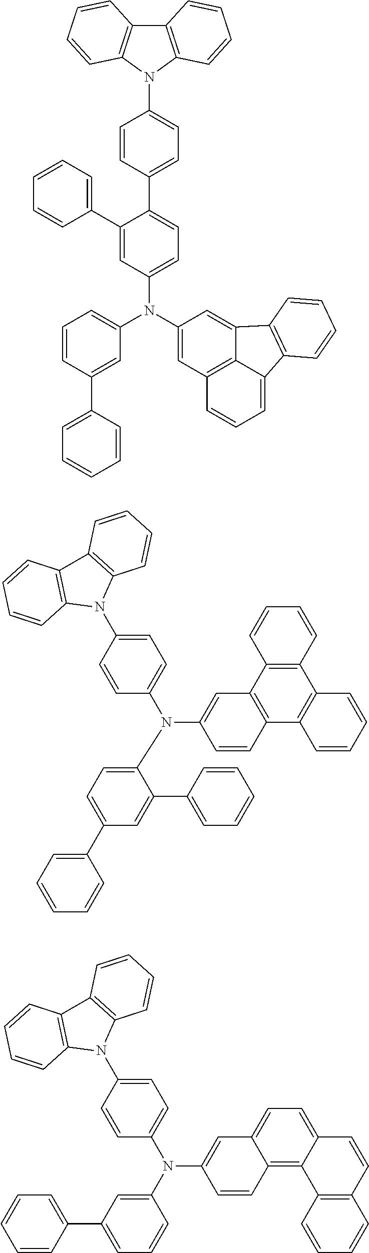

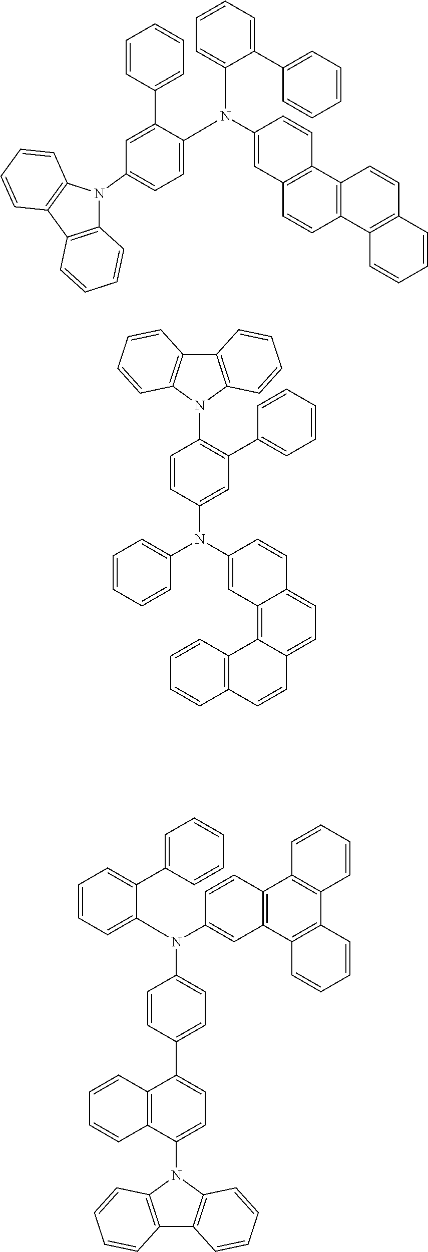 [SMILES: c1ccc(-c2ccccc2N(c2ccc3c(ccc4c5ccccc5ccc34)c2)c2ccc(-n3c4ccccc4c4ccccc43)cc2-c2ccccc2)cc1 ; c1ccc(-c2cc(N(c3ccccc3)c3ccc4ccc5ccc6ccccc6c5c4c3)ccc2-n2c3ccccc3c3ccccc32)cc1 ; c1ccc(-c2ccccc2N(c2ccc(-c3ccc(-n4c5ccccc5c5ccccc54)c4ccccc34)cc2)c2ccc3c4ccccc4c4ccccc4c3c2)cc1]

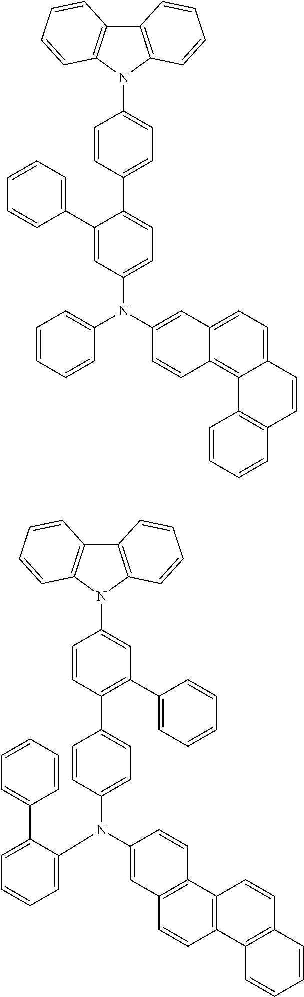 [SMILES: c1ccc(-c2cc(N(c3ccccc3)c3ccc4c(ccc5ccc6ccccc6c54)c3)ccc2-c2ccc(-n3c4ccccc4c4ccccc43)cc2)cc1 ; c1ccc(-c2cc(-n3c4ccccc4c4ccccc43)ccc2-c2ccc(N(c3ccc4c(ccc5c6ccccc6ccc45)c3)c3ccccc3-c3ccccc3)cc2)cc1]

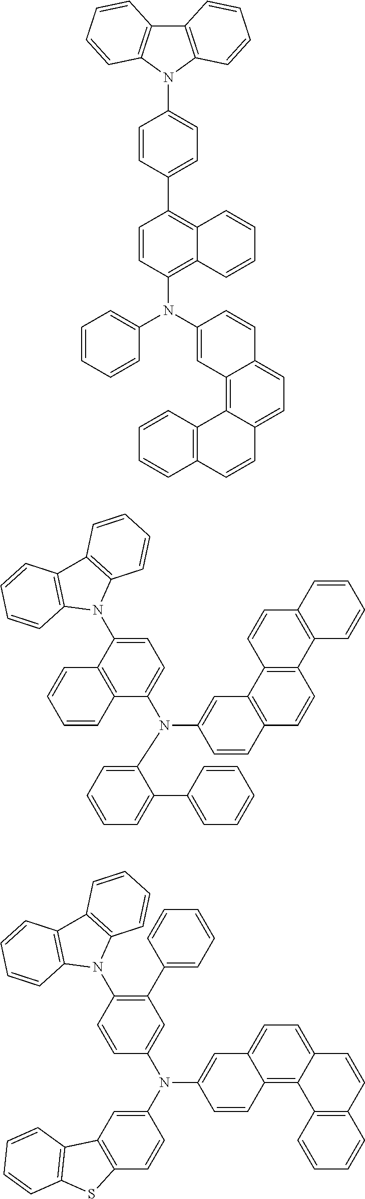 [SMILES: c1ccc(N(c2ccc3ccc4ccc5ccccc5c4c3c2)c2ccc(-c3ccc(-n4c5ccccc5c5ccccc54)cc3)c3ccccc23)cc1 ; c1ccc(-c2ccccc2N(c2ccc3ccc4c5ccccc5ccc4c3c2)c2ccc(-n3c4ccccc4c4ccccc43)c3ccccc23)cc1 ; c1ccc(-c2cc(N(c3ccc4c(ccc5ccc6ccccc6c54)c3)c3ccc4sc5ccccc5c4c3)ccc2-n2c3ccccc3c3ccccc32)cc1]

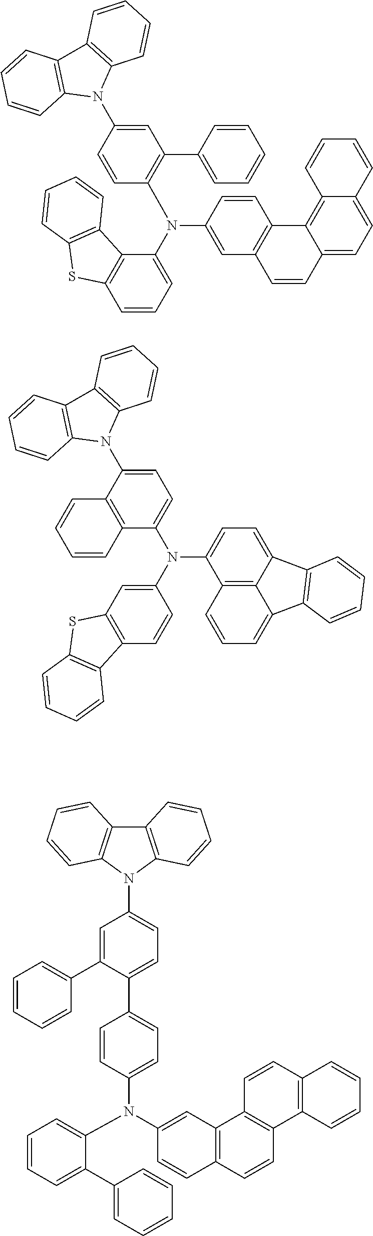

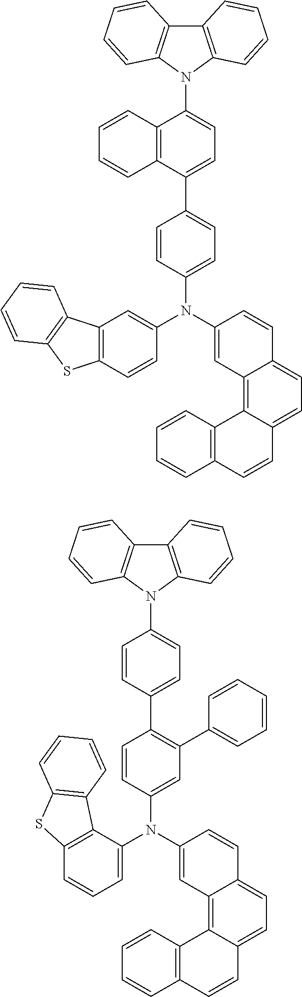

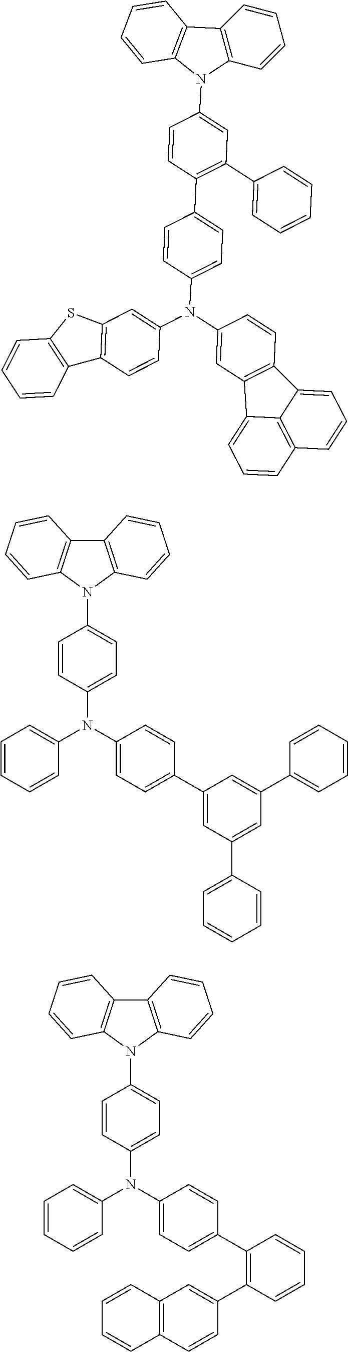 [SMILES: c1ccc(-c2cc(-n3c4ccccc4c4ccccc43)ccc2-c2ccc(N(c3ccc4c(c3)-c3cccc5cccc-4c35)c3ccc4c(c3)sc3ccccc34)cc2)cc1 ; c1ccc(-c2cc(-c3ccccc3)cc(-c3ccc(N(c4ccccc4)c4ccc(-n5c6ccccc6c6ccccc65)cc4)cc3)c2)cc1 ; c1ccc(N(c2ccc(-c3ccccc3-c3ccc4ccccc4c3)cc2)c2ccc(-n3c4ccccc4c4ccccc43)cc2)cc1]

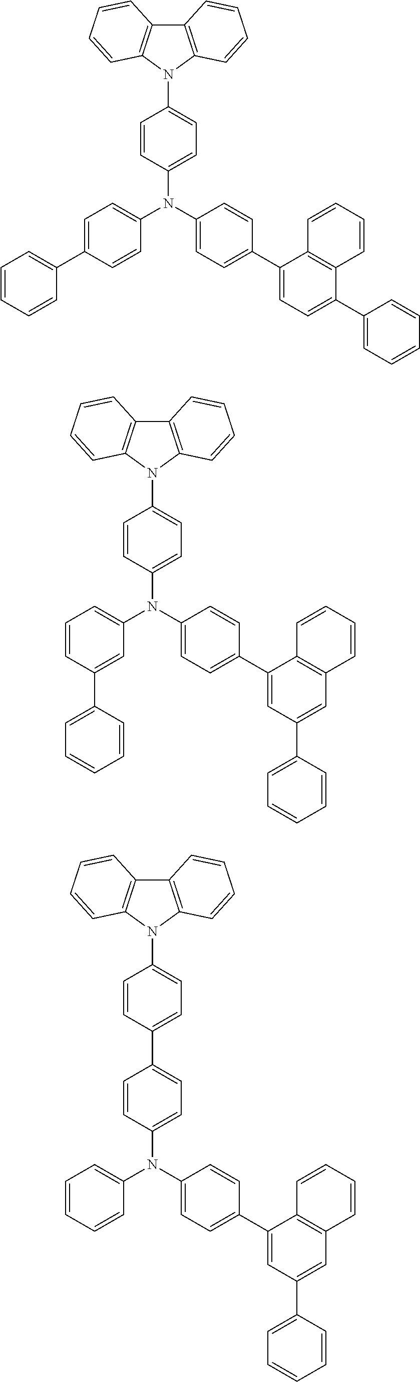 [SMILES: c1ccc(-c2ccc(N(c3ccc(-c4ccc(-c5ccccc5)c5ccccc45)cc3)c3ccc(-n4c5ccccc5c5ccccc54)cc3)cc2)cc1 ; c1ccc(-c2cccc(N(c3ccc(-c4cc(-c5ccccc5)cc5ccccc45)cc3)c3ccc(-n4c5ccccc5c5ccccc54)cc3)c2)cc1 ; c1ccc(-c2cc(-c3ccc(N(c4ccccc4)c4ccc(-c5ccc(-n6c7ccccc7c7ccccc76)cc5)cc4)cc3)c3ccccc3c2)cc1]

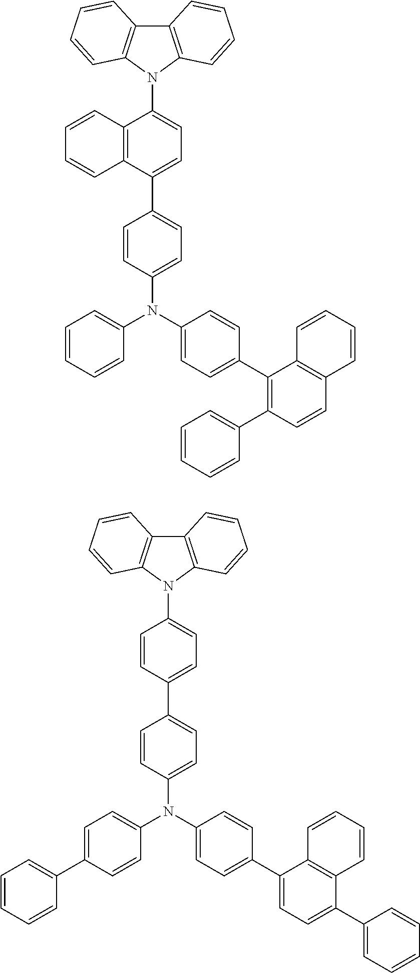

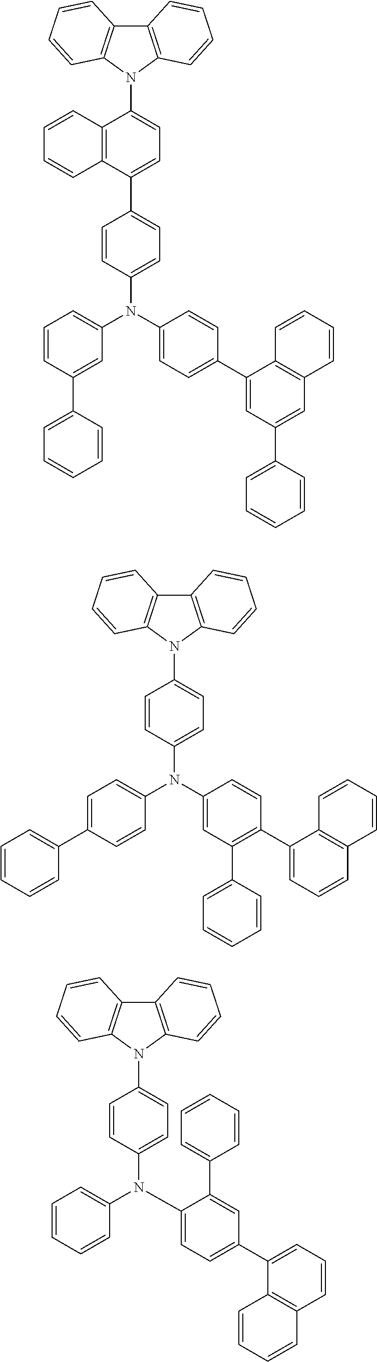

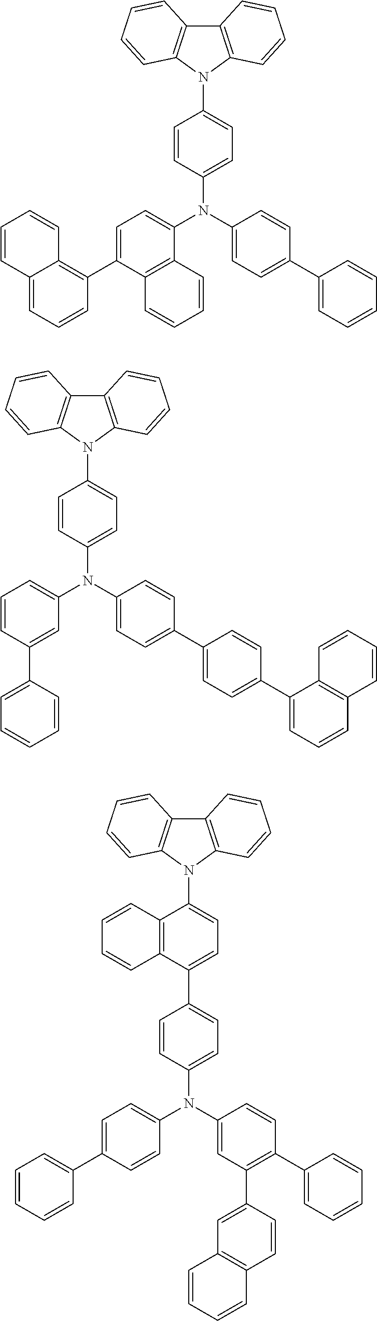

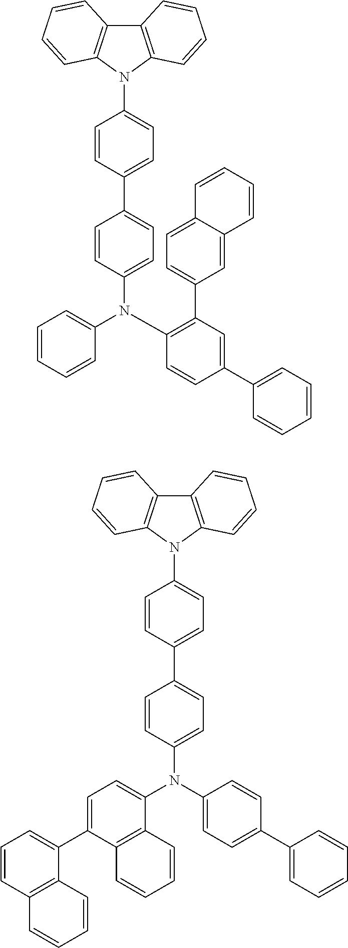 [SMILES: c1ccc(-c2ccc(N(c3ccccc3)c3ccc(-c4ccc(-n5c6ccccc6c6ccccc65)cc4)cc3)c(-c3ccc4ccccc4c3)c2)cc1 ; c1ccc(-c2ccc(N(c3ccc(-c4ccc(-n5c6ccccc6c6ccccc65)cc4)cc3)c3ccc(-c4cccc5ccccc45)c4ccccc34)cc2)cc1]

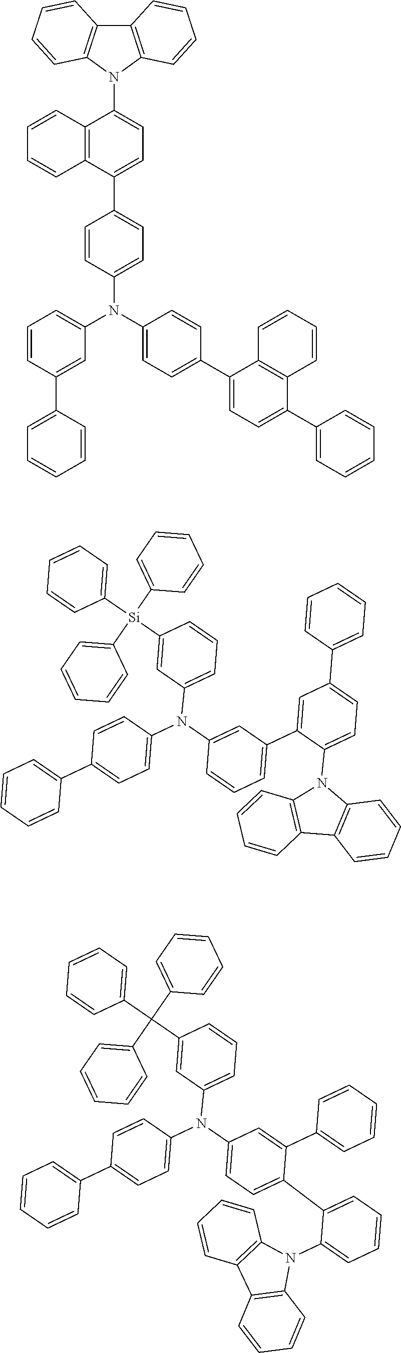 [SMILES: c1ccc(-c2cccc(N(c3ccc(-c4ccc(-c5ccccc5)c5ccccc45)cc3)c3ccc(-c4ccc(-n5c6ccccc6c6ccccc65)c5ccccc45)cc3)c2)cc1 ; c1ccc(-c2ccc(N(c3cccc(-c4cc(-c5ccccc5)ccc4-n4c5ccccc5c5ccccc54)c3)c3cccc([Si](c4ccccc4)(c4ccccc4)c4ccccc4)c3)cc2)cc1 ; c1ccc(-c2ccc(N(c3cccc(C(c4ccccc4)(c4ccccc4)c4ccccc4)c3)c3ccc(-c4ccccc4-n4c5ccccc5c5ccccc54)c(-c4ccccc4)c3)cc2)cc1]

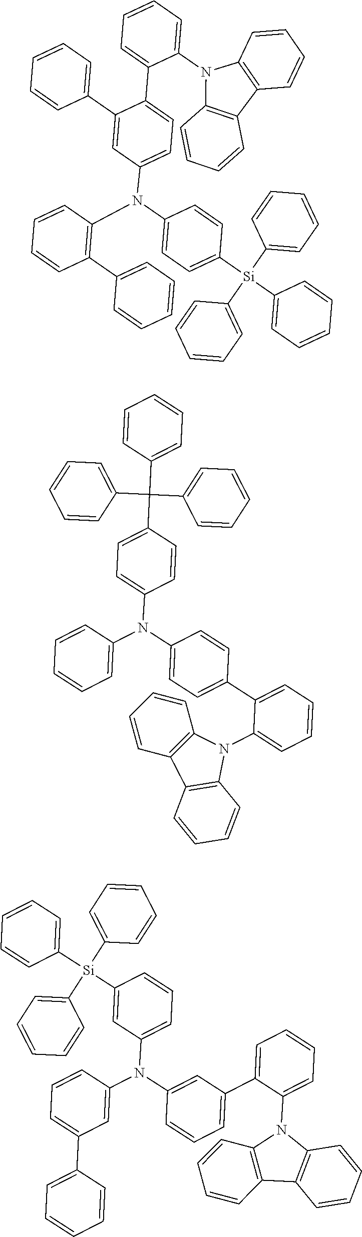 [SMILES: c1ccc(-c2cc(N(c3ccc([Si](c4ccccc4)(c4ccccc4)c4ccccc4)cc3)c3ccccc3-c3ccccc3)ccc2-c2ccccc2-n2c3ccccc3c3ccccc32)cc1 ; c1ccc(N(c2ccc(-c3ccccc3-n3c4ccccc4c4ccccc43)cc2)c2ccc(C(c3ccccc3)(c3ccccc3)c3ccccc3)cc2)cc1 ; c1ccc(-c2cccc(N(c3cccc(-c4ccccc4-n4c5ccccc5c5ccccc54)c3)c3cccc([Si](c4ccccc4)(c4ccccc4)c4ccccc4)c3)c2)cc1]

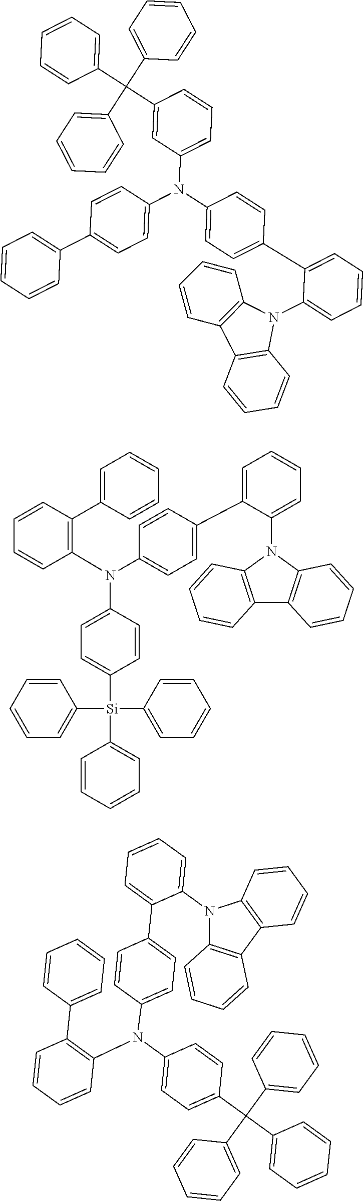

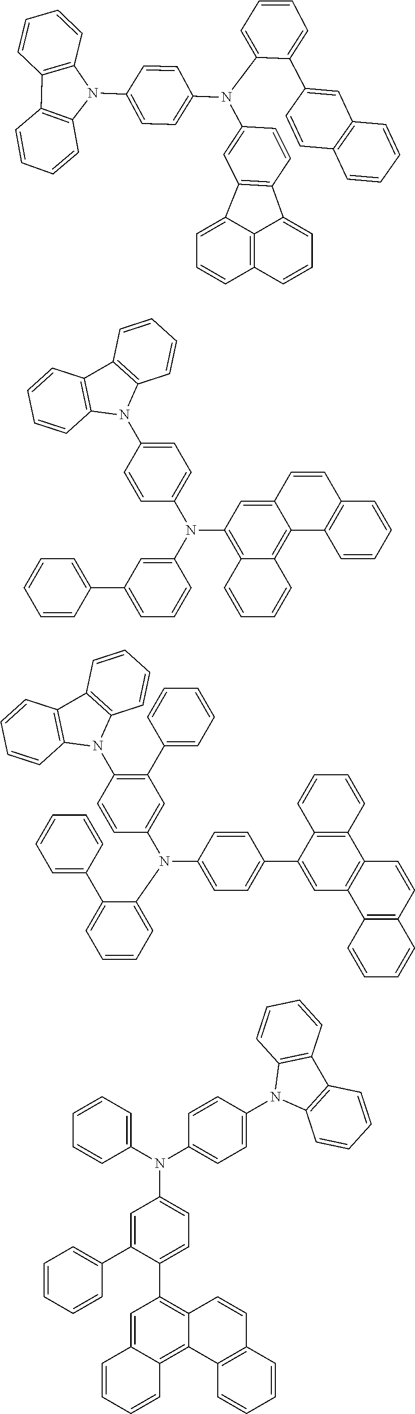

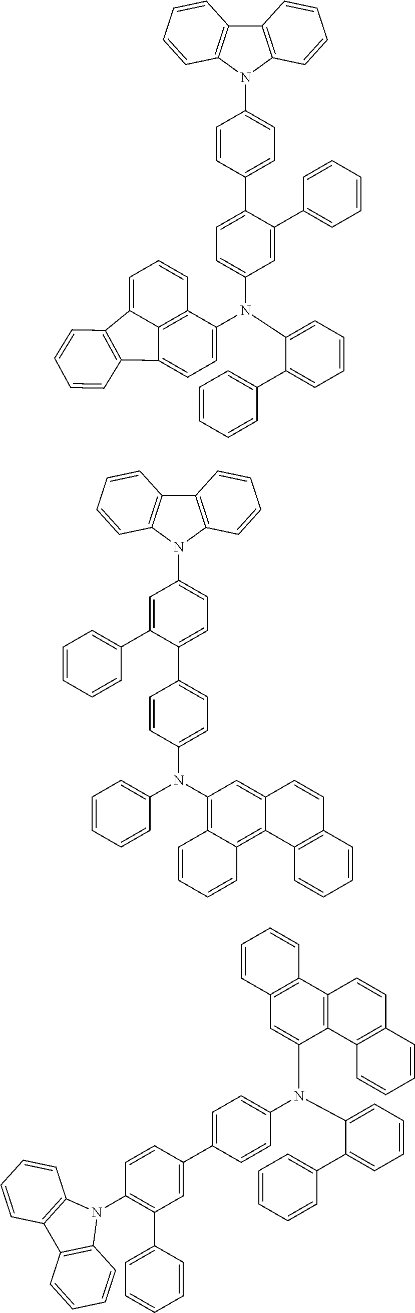

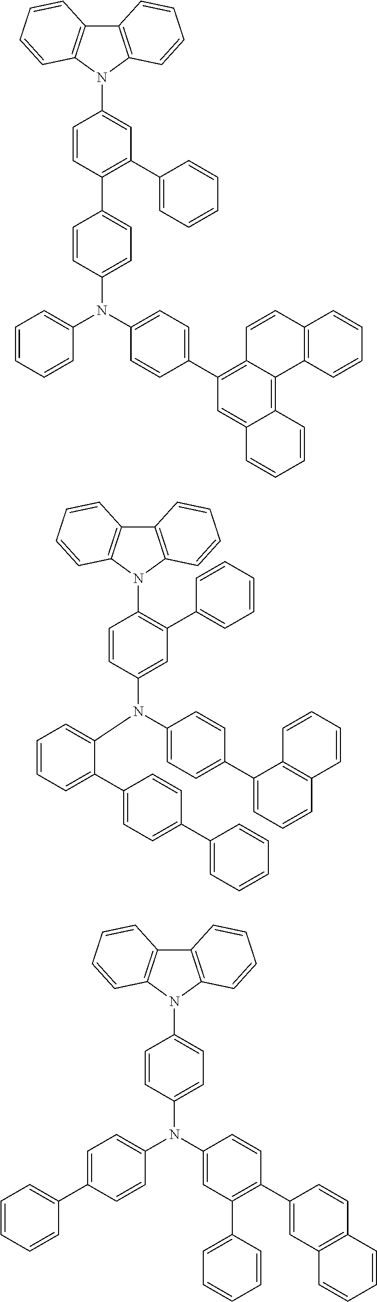

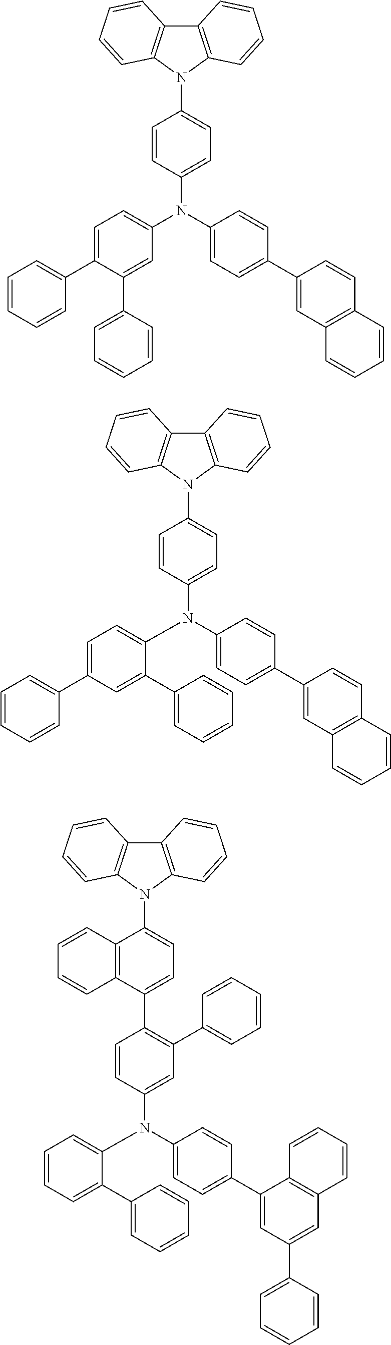

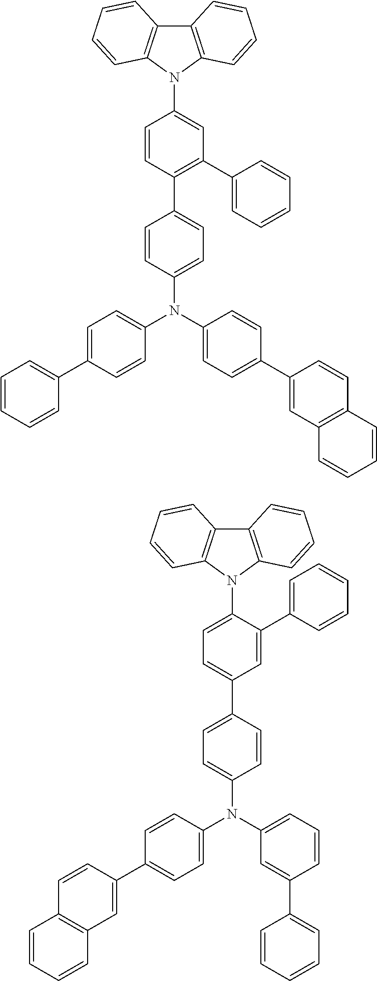 [SMILES: c1ccc(-c2ccc(N(c3ccc(-c4ccc5ccccc5c4)cc3)c3ccc(-c4ccc(-n5c6ccccc6c6ccccc65)cc4-c4ccccc4)cc3)cc2)cc1 ; c1ccc(-c2cccc(N(c3ccc(-c4ccc(-n5c6ccccc6c6ccccc65)c(-c5ccccc5)c4)cc3)c3ccc(-c4ccc5ccccc5c4)cc3)c2)cc1]

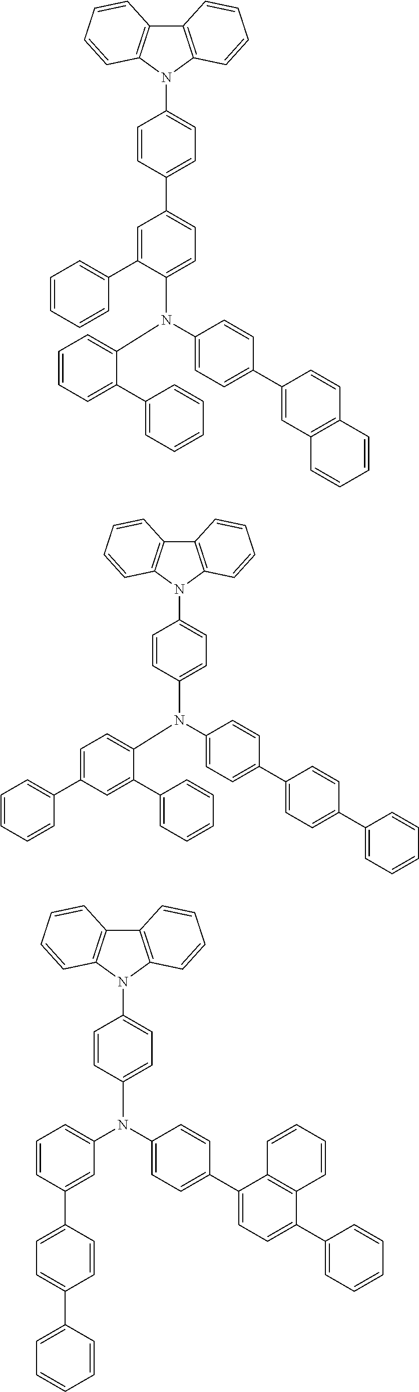 [SMILES: c1ccc(-c2ccccc2N(c2ccc(-c3ccc4ccccc4c3)cc2)c2ccc(-c3ccc(-n4c5ccccc5c5ccccc54)cc3)cc2-c2ccccc2)cc1 ; c1ccc(-c2ccc(-c3ccc(N(c4ccc(-n5c6ccccc6c6ccccc65)cc4)c4ccc(-c5ccccc5)cc4-c4ccccc4)cc3)cc2)cc1 ; c1ccc(-c2ccc(-c3cccc(N(c4ccc(-c5ccc(-c6ccccc6)c6ccccc56)cc4)c4ccc(-n5c6ccccc6c6ccccc65)cc4)c3)cc2)cc1]

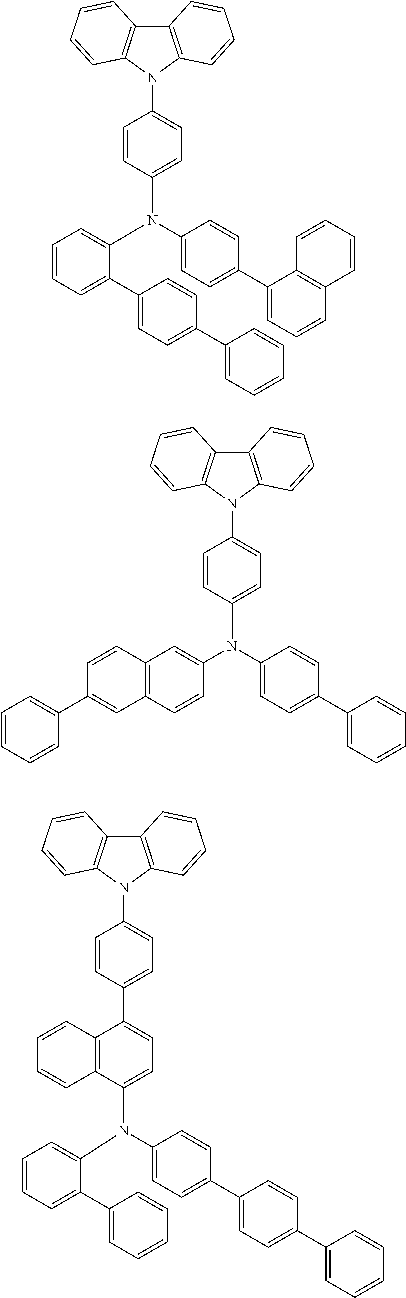

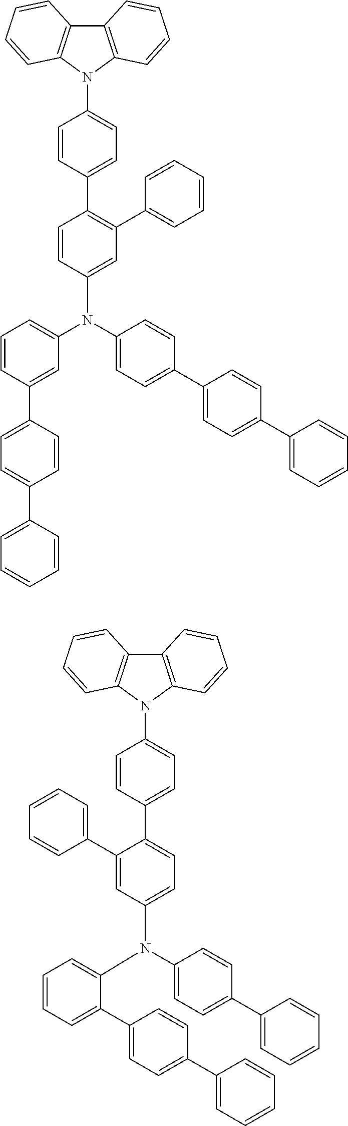 [SMILES: c1ccc(-c2ccc(-c3ccc(N(c4cccc(-c5ccc(-c6ccccc6)cc5)c4)c4ccc(-c5ccc(-n6c7ccccc7c7ccccc76)cc5)c(-c5ccccc5)c4)cc3)cc2)cc1 ; c1ccc(-c2ccc(-c3ccccc3N(c3ccc(-c4ccccc4)cc3)c3ccc(-c4ccc(-n5c6ccccc6c6ccccc65)cc4)c(-c4ccccc4)c3)cc2)cc1]

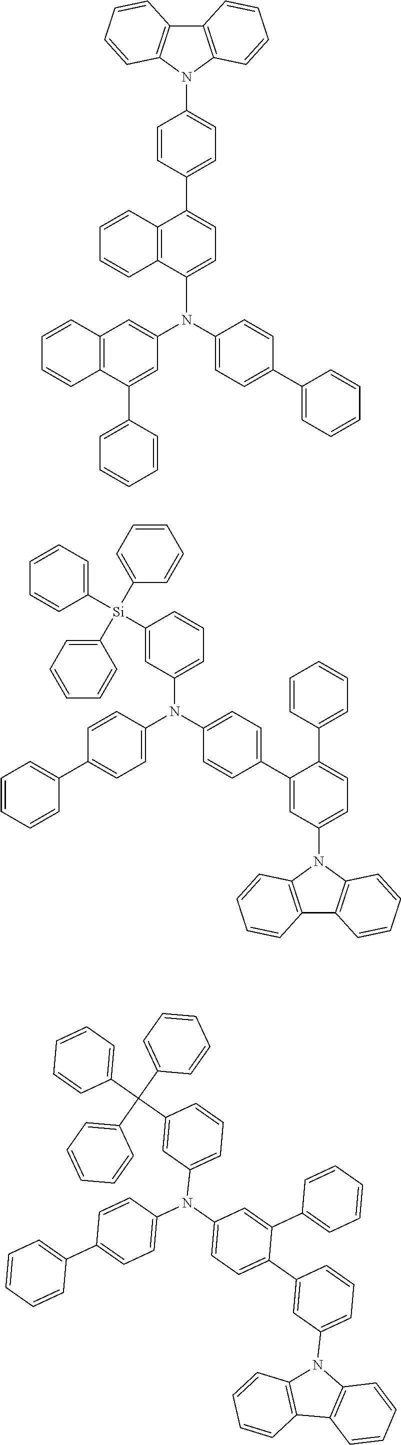

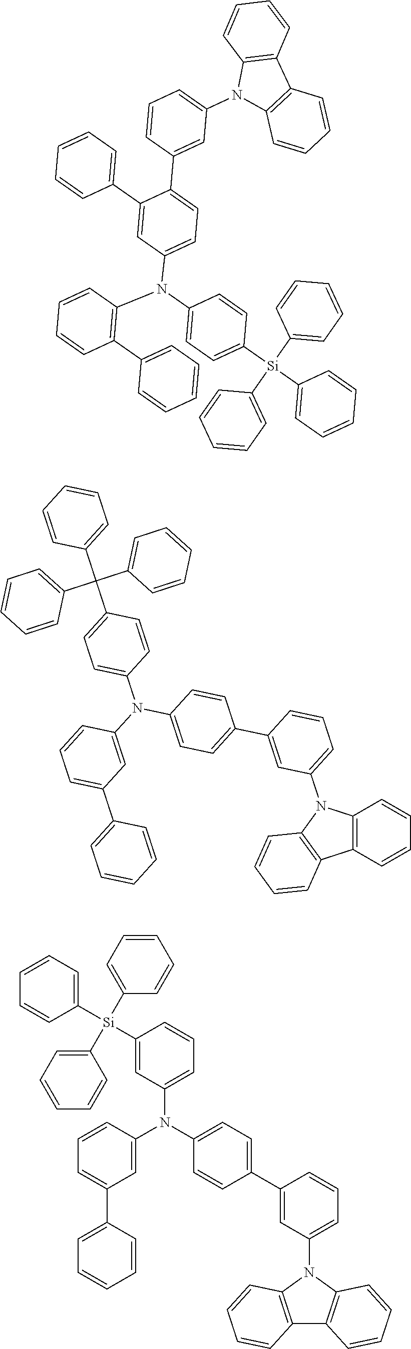 [SMILES: c1ccc(-c2cc(N(c3ccc([Si](c4ccccc4)(c4ccccc4)c4ccccc4)cc3)c3ccccc3-c3ccccc3)ccc2-c2cccc(-n3c4ccccc4c4ccccc43)c2)cc1 ; c1ccc(-c2cccc(N(c3ccc(-c4cccc(-n5c6ccccc6c6ccccc65)c4)cc3)c3ccc(C(c4ccccc4)(c4ccccc4)c4ccccc4)cc3)c2)cc1 ; c1ccc(-c2cccc(N(c3ccc(-c4cccc(-n5c6ccccc6c6ccccc65)c4)cc3)c3cccc([Si](c4ccccc4)(c4ccccc4)c4ccccc4)c3)c2)cc1]

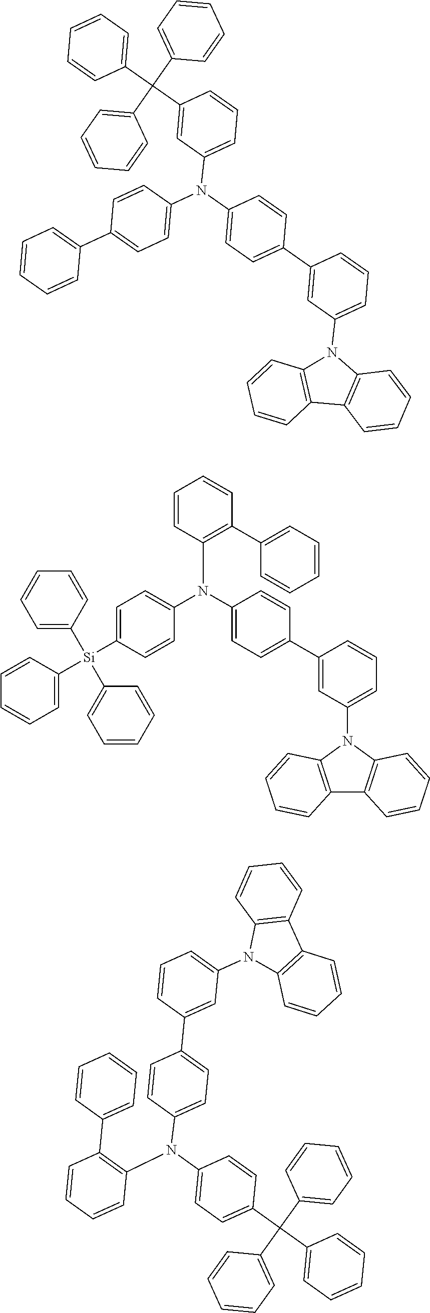 [SMILES: c1ccc(-c2ccc(N(c3ccc(-c4cccc(-n5c6ccccc6c6ccccc65)c4)cc3)c3cccc(C(c4ccccc4)(c4ccccc4)c4ccccc4)c3)cc2)cc1 ; c1ccc(-c2ccccc2N(c2ccc(-c3cccc(-n4c5ccccc5c5ccccc54)c3)cc2)c2ccc([Si](c3ccccc3)(c3ccccc3)c3ccccc3)cc2)cc1 ; c1ccc(-c2ccccc2N(c2ccc(-c3cccc(-n4c5ccccc5c5ccccc54)c3)cc2)c2ccc(C(c3ccccc3)(c3ccccc3)c3ccccc3)cc2)cc1]

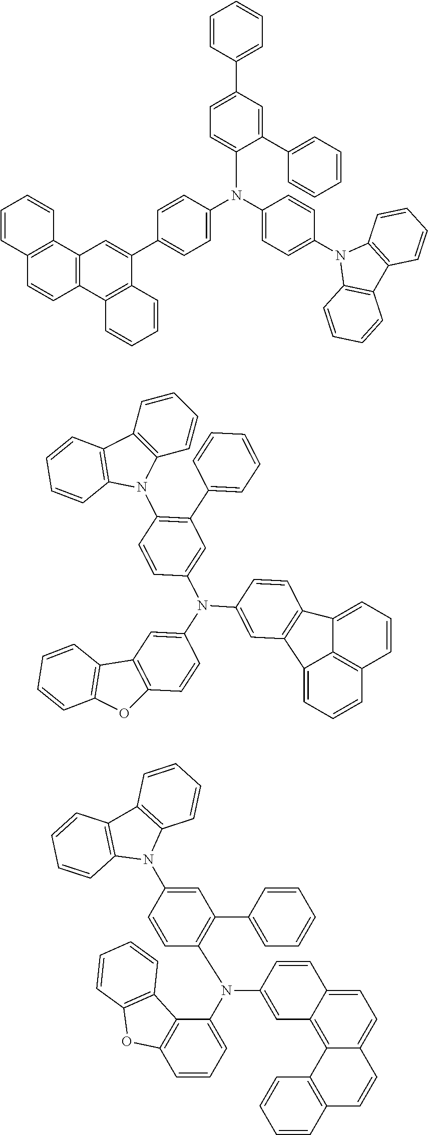 [SMILES: c1ccc(-c2ccc(N(c3ccc(-c4cc5c6ccccc6ccc5c5ccccc45)cc3)c3ccc(-n4c5ccccc5c5ccccc54)cc3)c(-c3ccccc3)c2)cc1 ; c1ccc(-c2cc(N(c3ccc4c(c3)-c3cccc5cccc-4c35)c3ccc4oc5ccccc5c4c3)ccc2-n2c3ccccc3c3ccccc32)cc1 ; c1ccc(-c2cc(-n3c4ccccc4c4ccccc43)ccc2N(c2ccc3ccc4ccc5ccccc5c4c3c2)c2cccc3oc4ccccc4c23)cc1]

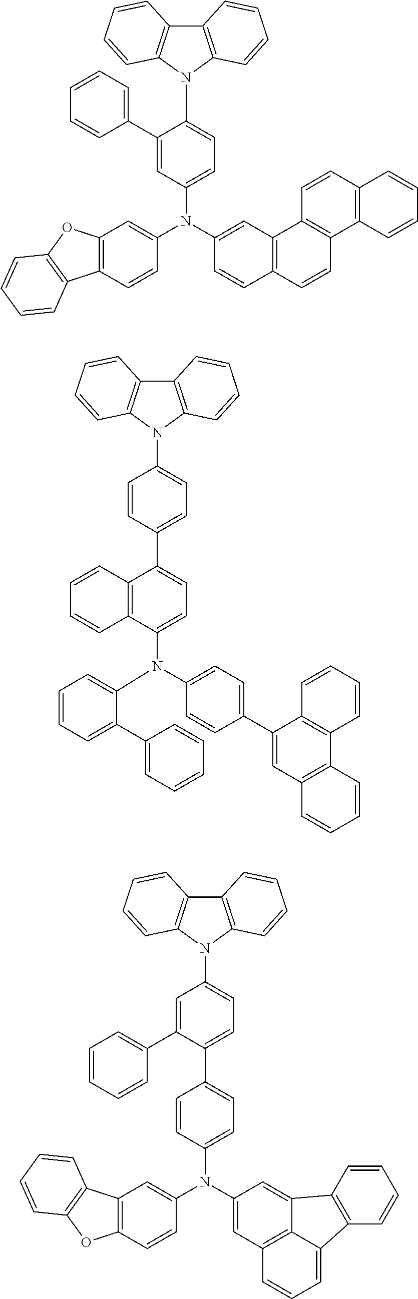 [SMILES: c1ccc(-c2cc(N(c3ccc4c(c3)oc3ccccc34)c3ccc4ccc5c6ccccc6ccc5c4c3)ccc2-n2c3ccccc3c3ccccc32)cc1 ; c1ccc(-c2ccccc2N(c2ccc(-c3cc4ccccc4c4ccccc34)cc2)c2ccc(-c3ccc(-n4c5ccccc5c5ccccc54)cc3)c3ccccc23)cc1 ; c1ccc(-c2cc(-n3c4ccccc4c4ccccc43)ccc2-c2ccc(N(c3cc4c5c(cccc5c3)-c3ccccc3-4)c3ccc4oc5ccccc5c4c3)cc2)cc1]

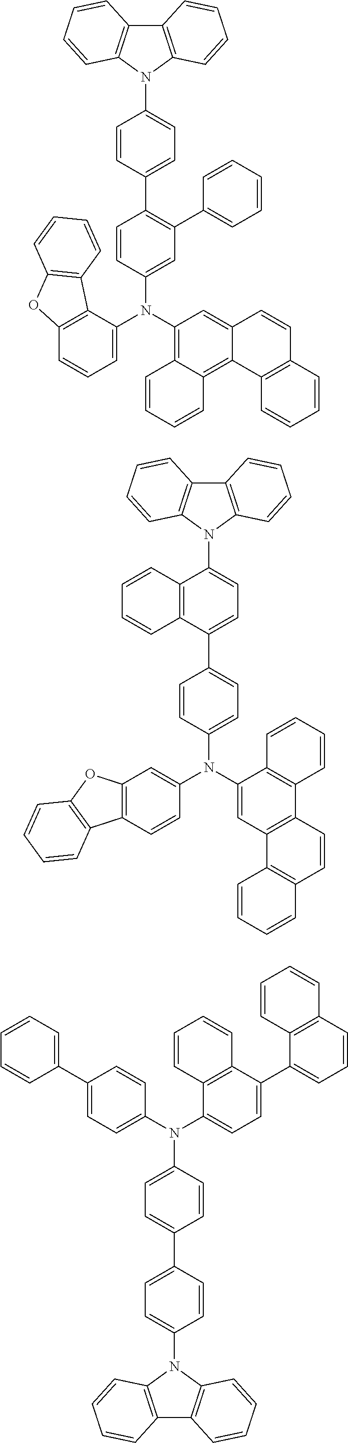

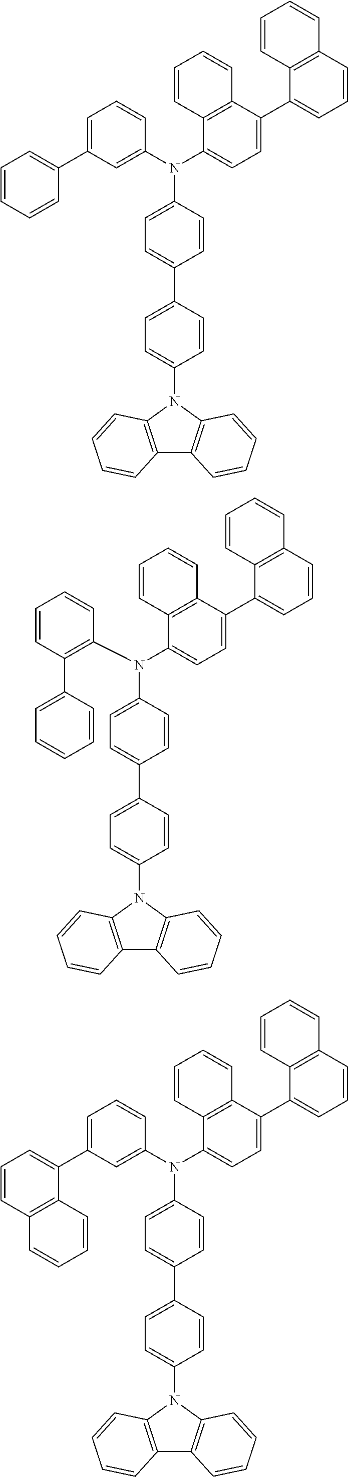

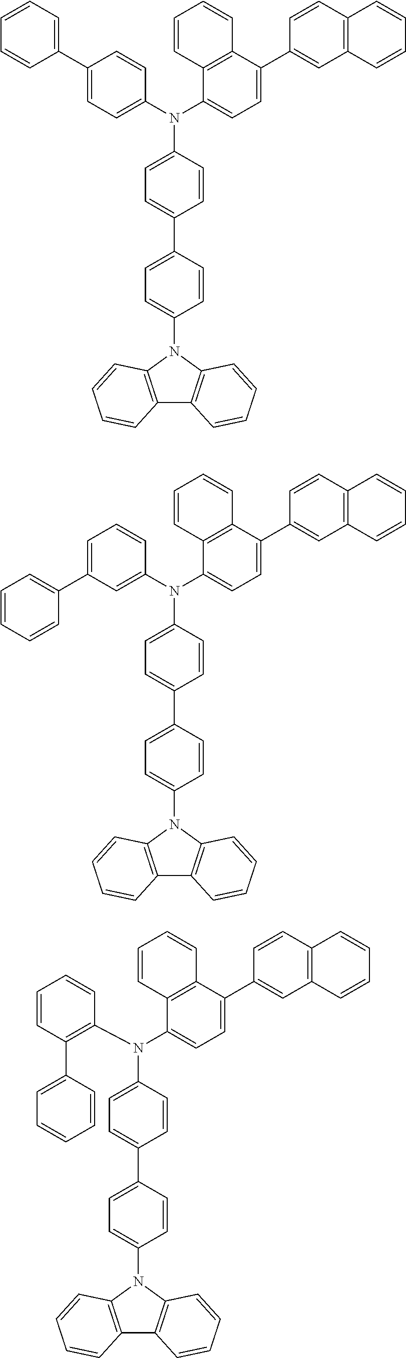

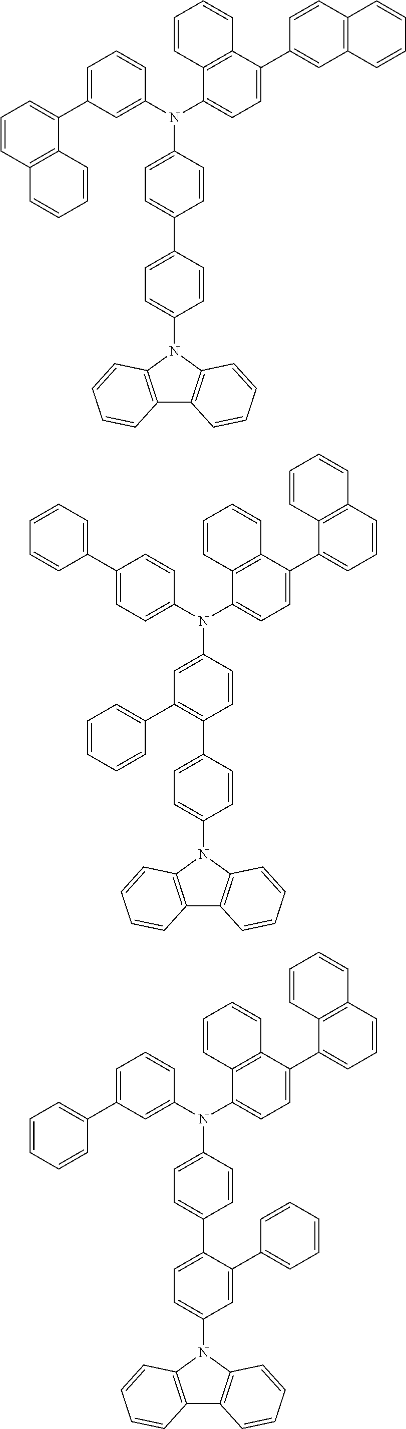 [SMILES: c1cc(-c2cccc3ccccc23)cc(N(c2ccc(-c3ccc(-n4c5ccccc5c5ccccc54)cc3)cc2)c2ccc(-c3ccc4ccccc4c3)c3ccccc23)c1 ; c1ccc(-c2ccc(N(c3ccc(-c4ccc(-n5c6ccccc6c6ccccc65)cc4)c(-c4ccccc4)c3)c3ccc(-c4cccc5ccccc45)c4ccccc34)cc2)cc1 ; c1ccc(-c2cccc(N(c3ccc(-c4ccc(-n5c6ccccc6c6ccccc65)cc4-c4ccccc4)cc3)c3ccc(-c4cccc5ccccc45)c4ccccc34)c2)cc1]

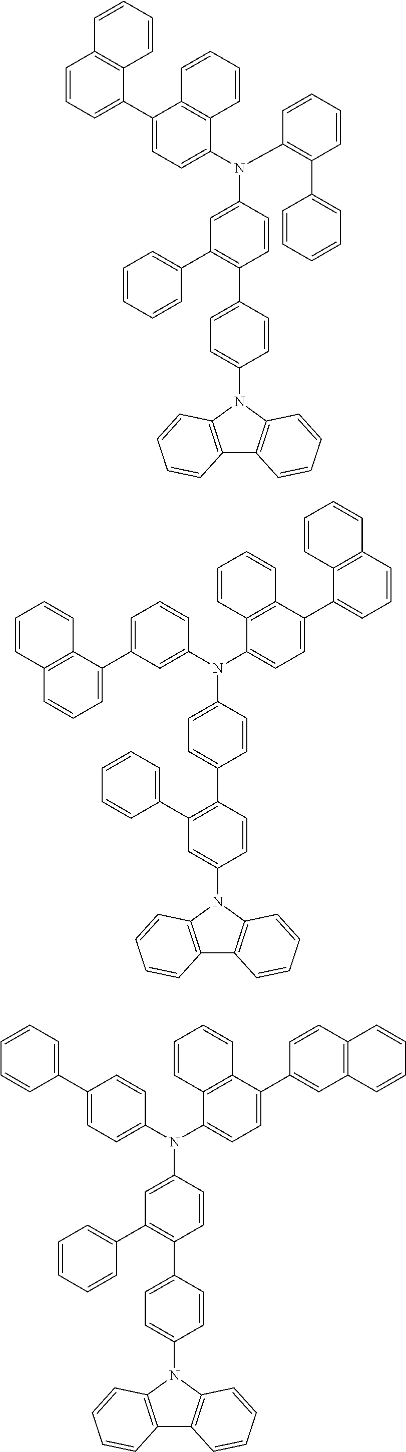

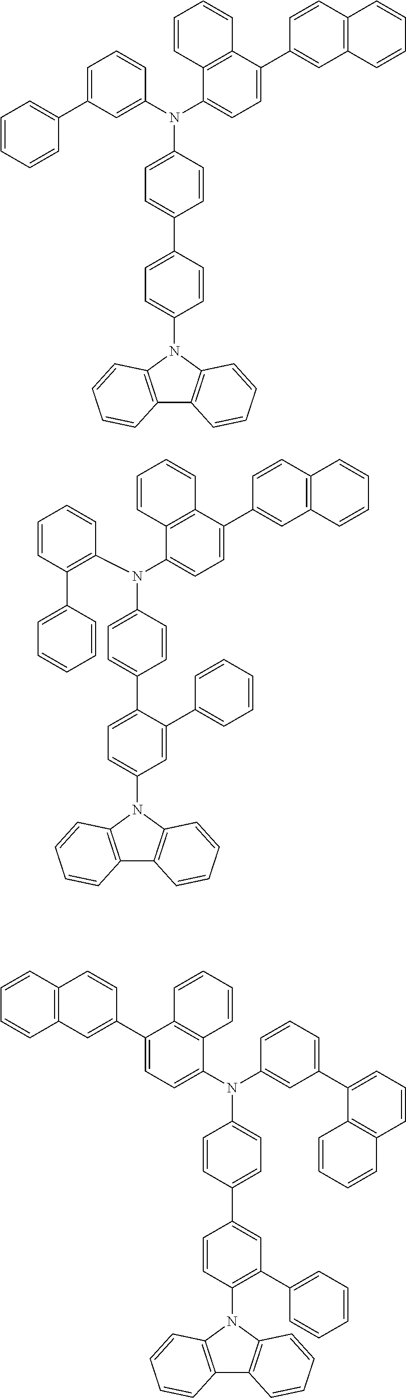

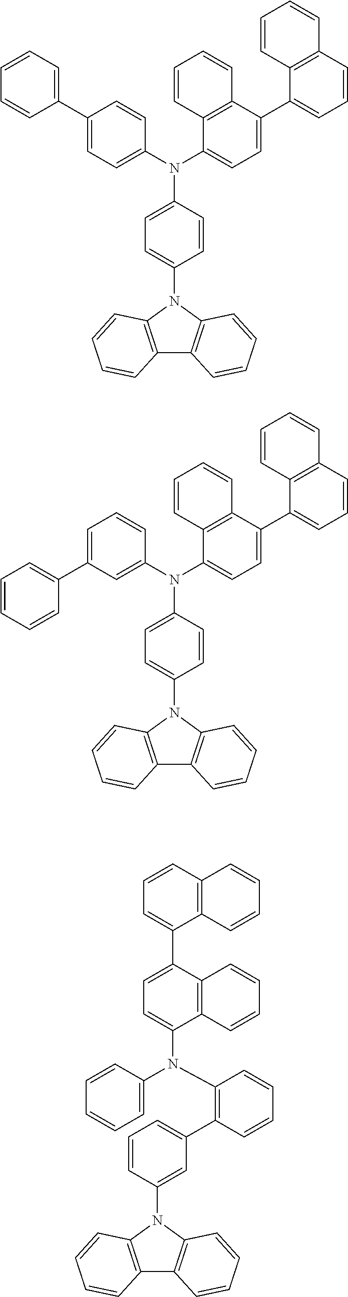 [SMILES: c1ccc(-c2ccc(N(c3ccc(-n4c5ccccc5c5ccccc54)cc3)c3ccc(-c4cccc5ccccc45)c4ccccc34)cc2)cc1 ; c1ccc(-c2cccc(N(c3ccc(-n4c5ccccc5c5ccccc54)cc3)c3ccc(-c4cccc5ccccc45)c4ccccc34)c2)cc1 ; c1ccc(N(c2ccccc2-c2cccc(-n3c4ccccc4c4ccccc43)c2)c2ccc(-c3cccc4ccccc34)c3ccccc23)cc1]

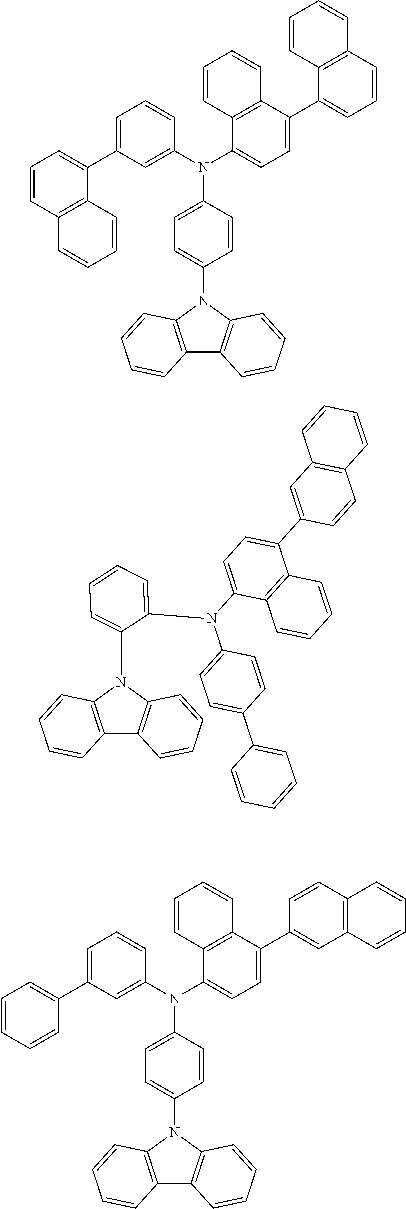 [SMILES: c1cc(-c2cccc3ccccc23)cc(N(c2ccc(-n3c4ccccc4c4ccccc43)cc2)c2ccc(-c3cccc4ccccc34)c3ccccc23)c1 ; c1ccc(-c2ccc(N(c3ccccc3-n3c4ccccc4c4ccccc43)c3ccc(-c4ccc5ccccc5c4)c4ccccc34)cc2)cc1 ; c1ccc(-c2cccc(N(c3ccc(-n4c5ccccc5c5ccccc54)cc3)c3ccc(-c4ccc5ccccc5c4)c4ccccc34)c2)cc1]

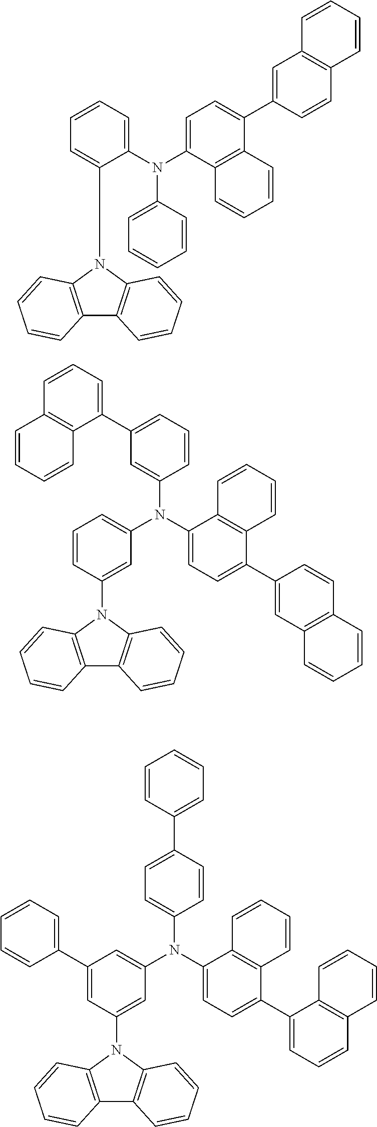

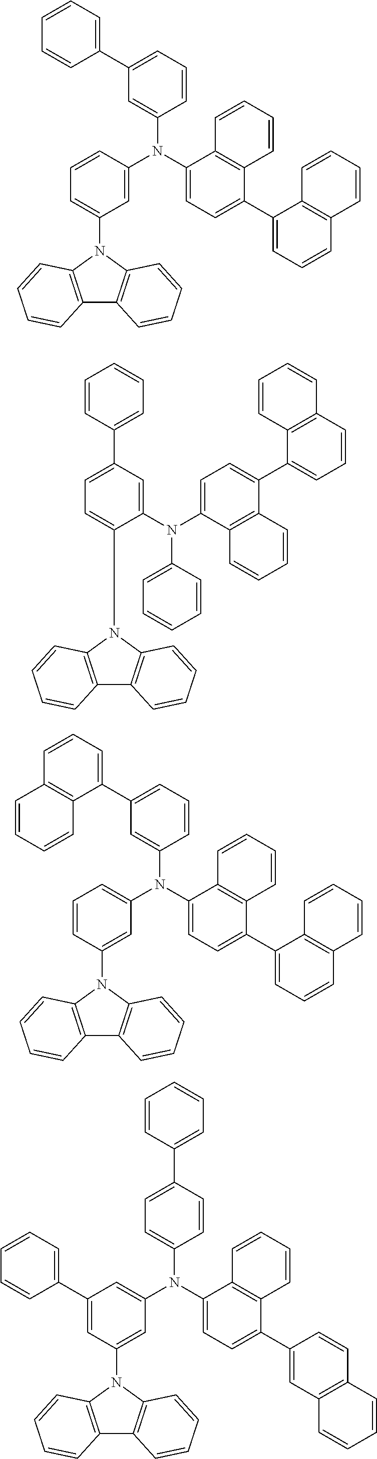

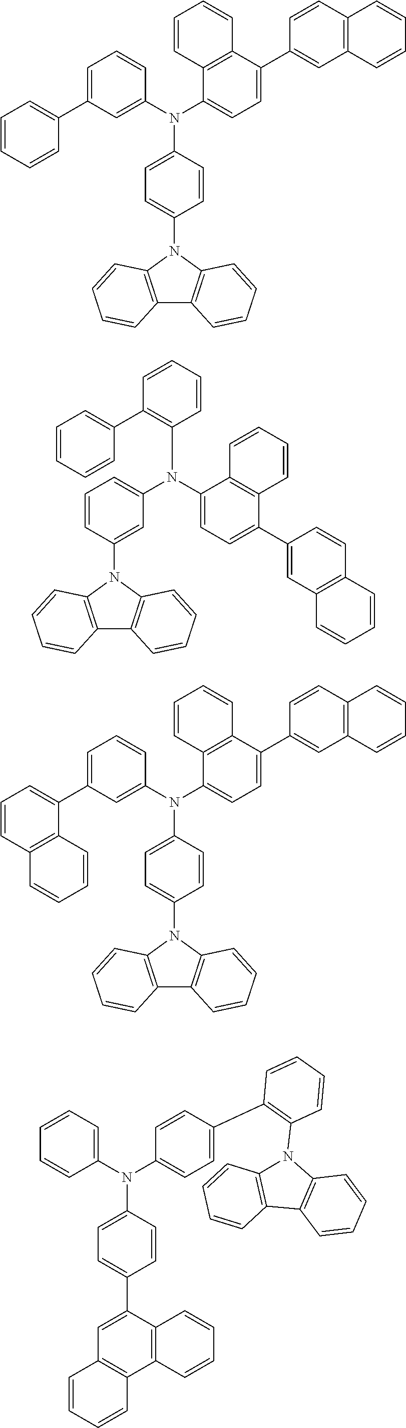

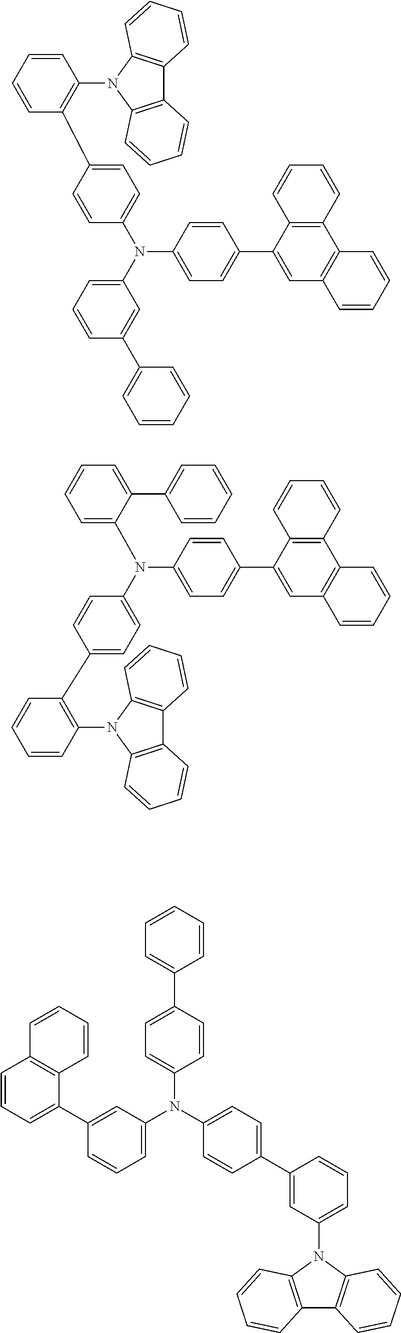

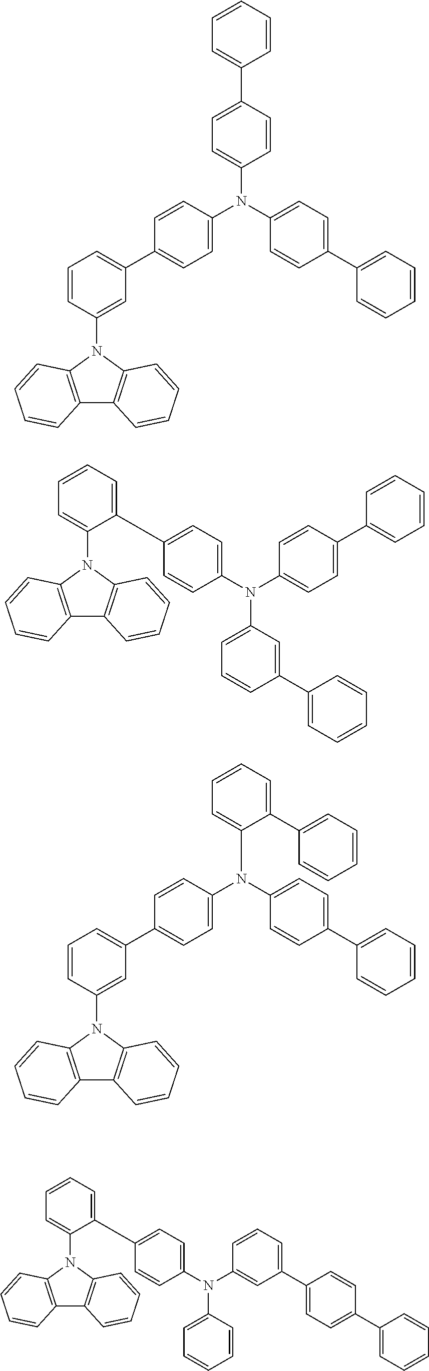 [SMILES: c1ccc(-c2ccc(N(c3ccc(-c4ccccc4)cc3)c3ccc(-c4cccc(-n5c6ccccc6c6ccccc65)c4)cc3)cc2)cc1 ; c1ccc(-c2ccc(N(c3ccc(-c4ccccc4-n4c5ccccc5c5ccccc54)cc3)c3cccc(-c4ccccc4)c3)cc2)cc1 ; c1ccc(-c2ccc(N(c3ccc(-c4cccc(-n5c6ccccc6c6ccccc65)c4)cc3)c3ccccc3-c3ccccc3)cc2)cc1 ; c1ccc(-c2ccc(-c3cccc(N(c4ccccc4)c4ccc(-c5ccccc5-n5c6ccccc6c6ccccc65)cc4)c3)cc2)cc1]

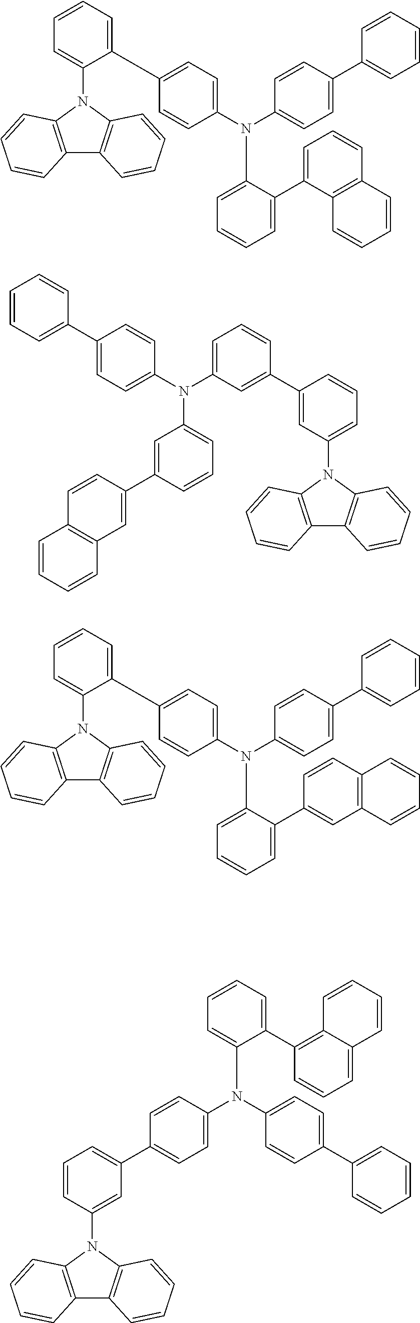

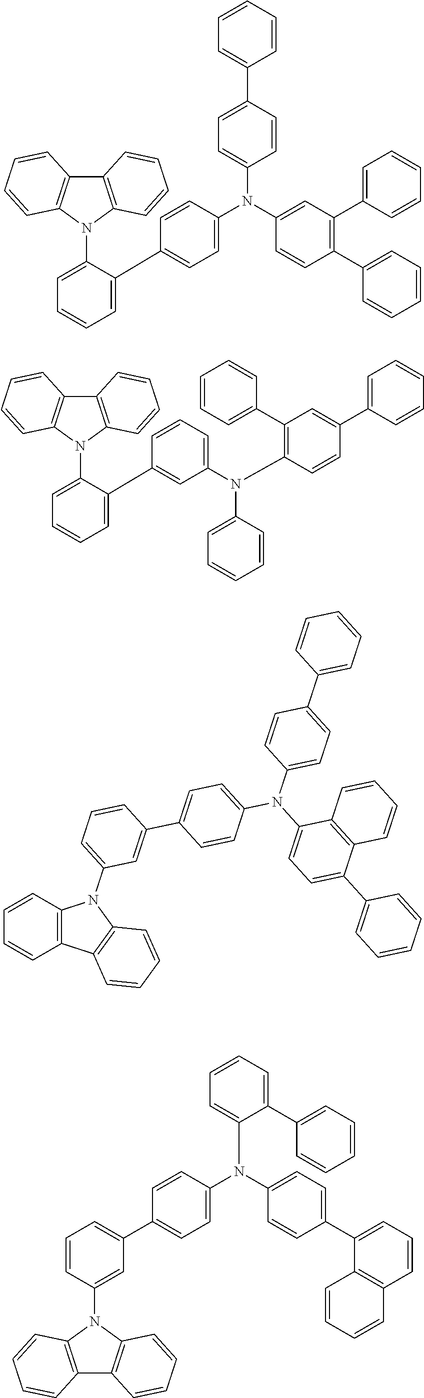

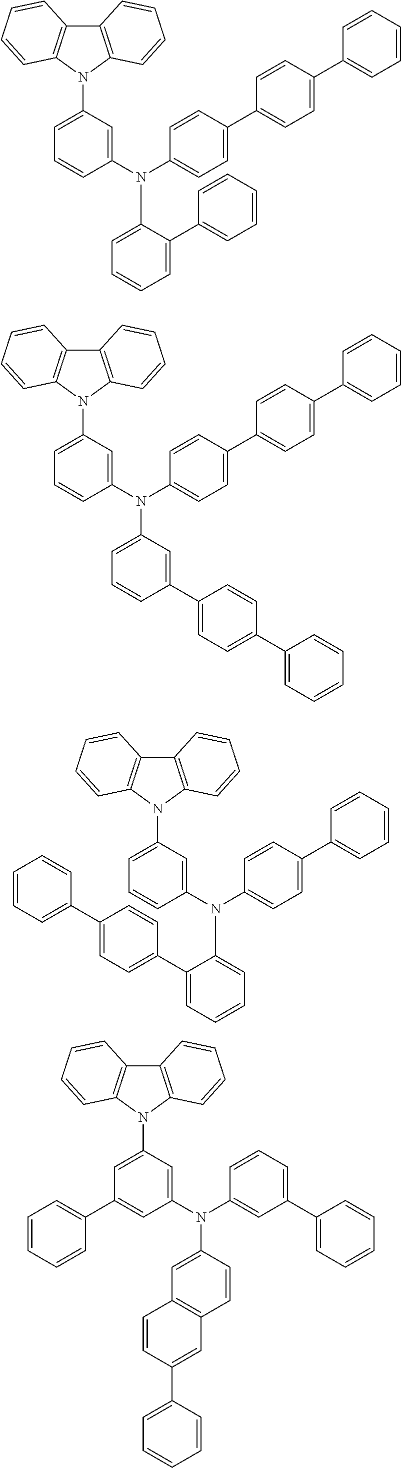

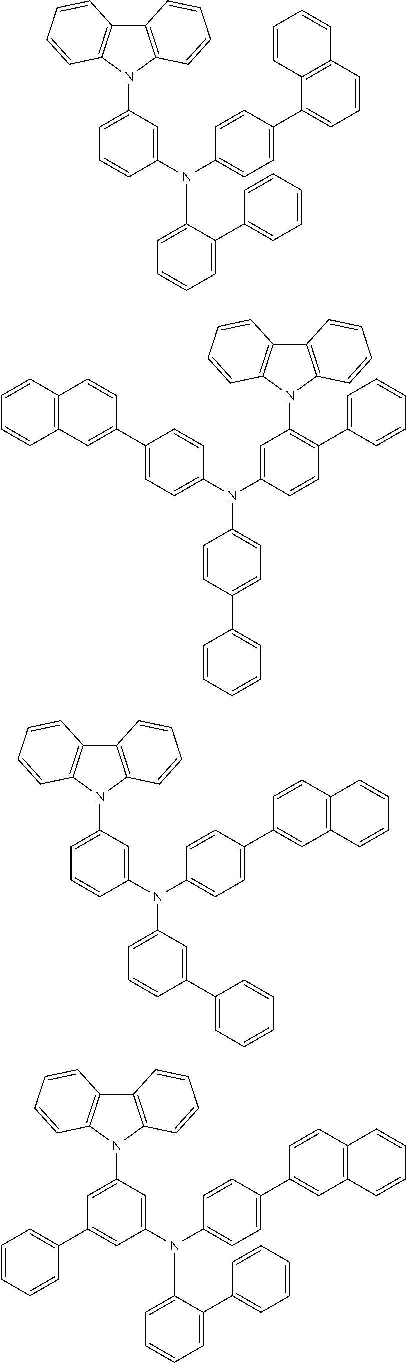 [SMILES: c1ccc(-c2ccccc2N(c2ccc(-c3cccc4ccccc34)cc2)c2cccc(-n3c4ccccc4c4ccccc43)c2)cc1 ; c1ccc(-c2ccc(N(c3ccc(-c4ccc5ccccc5c4)cc3)c3ccc(-c4ccccc4)c(-n4c5ccccc5c5ccccc54)c3)cc2)cc1 ; c1ccc(-c2cccc(N(c3ccc(-c4ccc5ccccc5c4)cc3)c3cccc(-n4c5ccccc5c5ccccc54)c3)c2)cc1 ; c1ccc(-c2cc(N(c3ccc(-c4ccc5ccccc5c4)cc3)c3ccccc3-c3ccccc3)cc(-n3c4ccccc4c4ccccc43)c2)cc1]

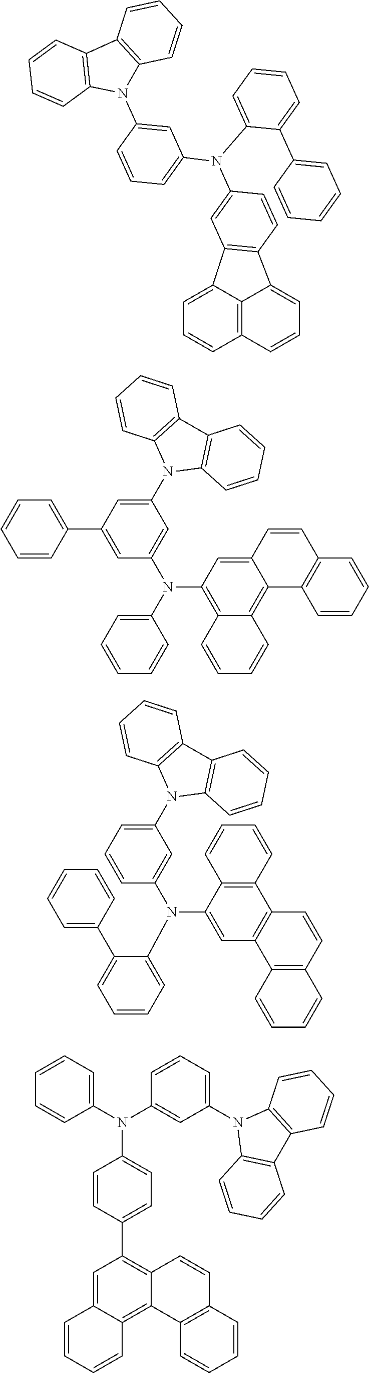

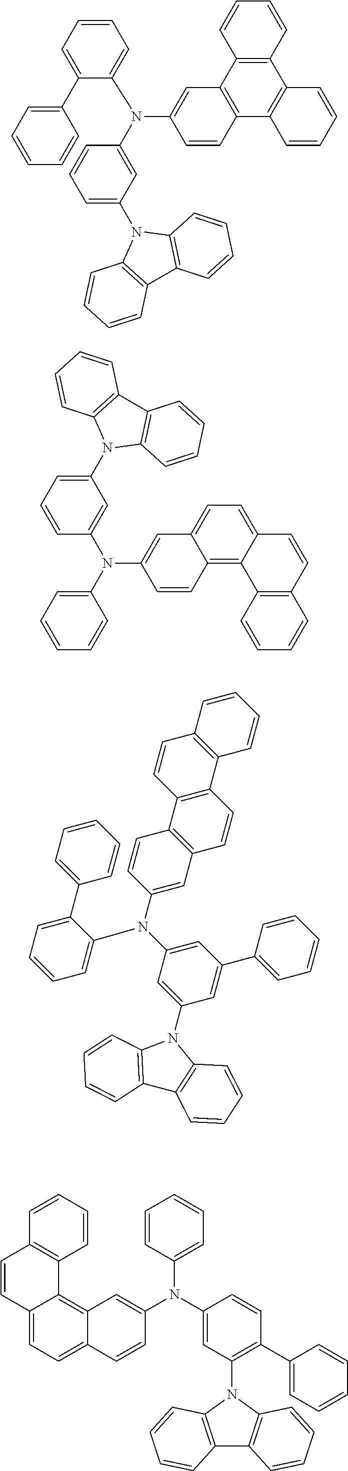

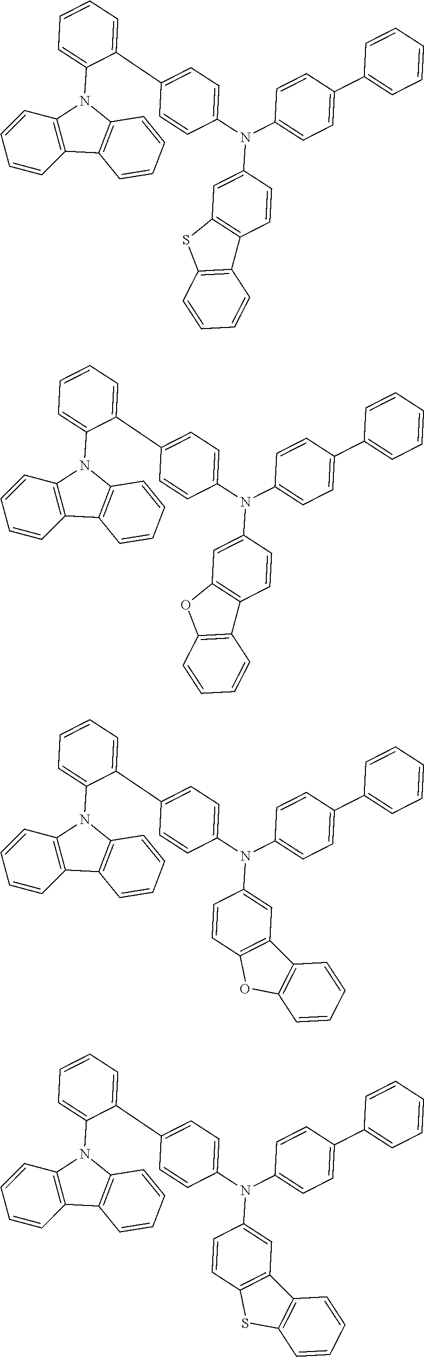 [SMILES: c1ccc(-c2ccc(N(c3ccc(-c4ccccc4-n4c5ccccc5c5ccccc54)cc3)c3ccc4c(c3)sc3ccccc34)cc2)cc1 ; c1ccc(-c2ccc(N(c3ccc(-c4ccccc4-n4c5ccccc5c5ccccc54)cc3)c3ccc4c(c3)oc3ccccc34)cc2)cc1 ; c1ccc(-c2ccc(N(c3ccc(-c4ccccc4-n4c5ccccc5c5ccccc54)cc3)c3ccc4oc5ccccc5c4c3)cc2)cc1 ; c1ccc(-c2ccc(N(c3ccc(-c4ccccc4-n4c5ccccc5c5ccccc54)cc3)c3ccc4sc5ccccc5c4c3)cc2)cc1]

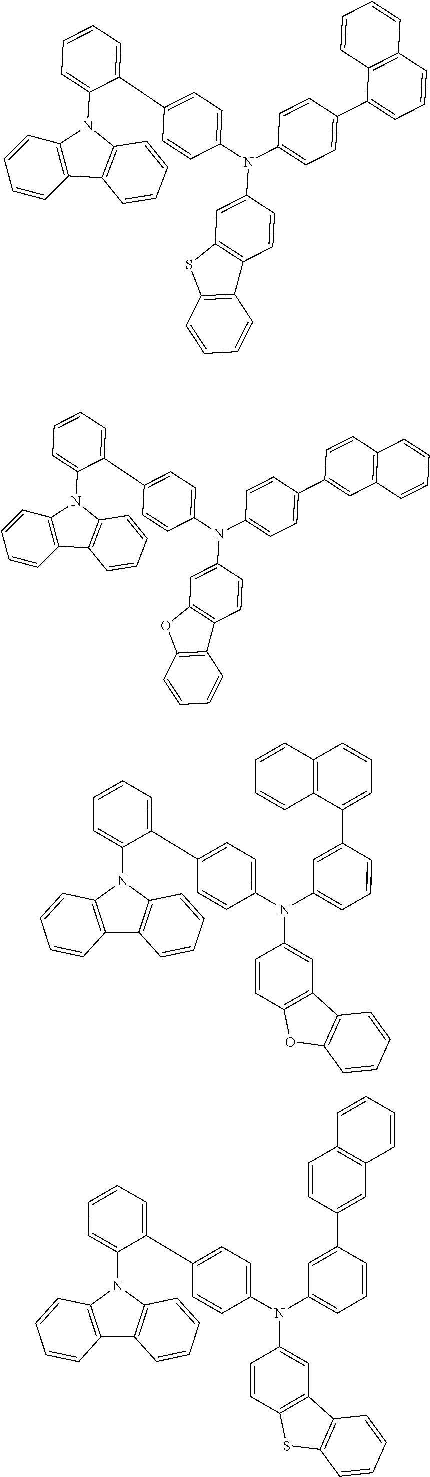

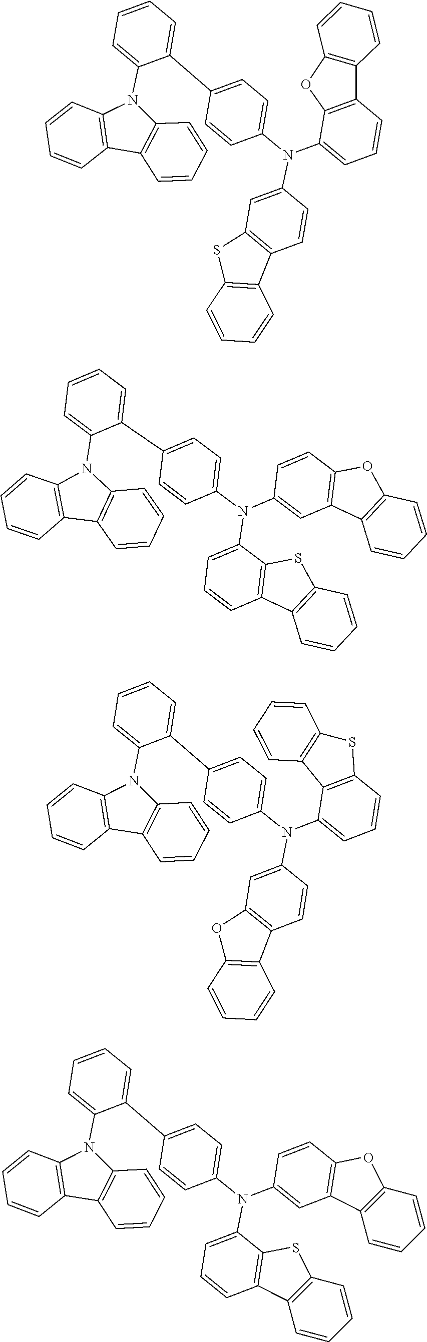 [SMILES: c1ccc(-n2c3ccccc3c3ccccc32)c(-c2ccc(N(c3ccc4c(c3)sc3ccccc34)c3cccc4c3oc3ccccc34)cc2)c1 ; c1ccc(-n2c3ccccc3c3ccccc32)c(-c2ccc(N(c3ccc4oc5ccccc5c4c3)c3cccc4c3sc3ccccc34)cc2)c1 ; c1ccc(-n2c3ccccc3c3ccccc32)c(-c2ccc(N(c3ccc4c(c3)oc3ccccc34)c3cccc4sc5ccccc5c34)cc2)c1 ; c1ccc(-n2c3ccccc3c3ccccc32)c(-c2ccc(N(c3ccc4oc5ccccc5c4c3)c3cccc4c3sc3ccccc34)cc2)c1]

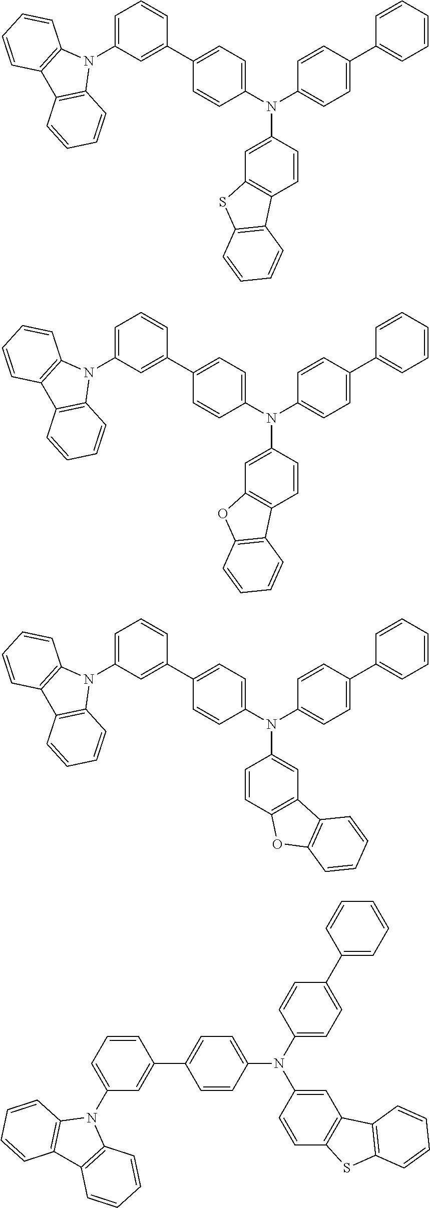

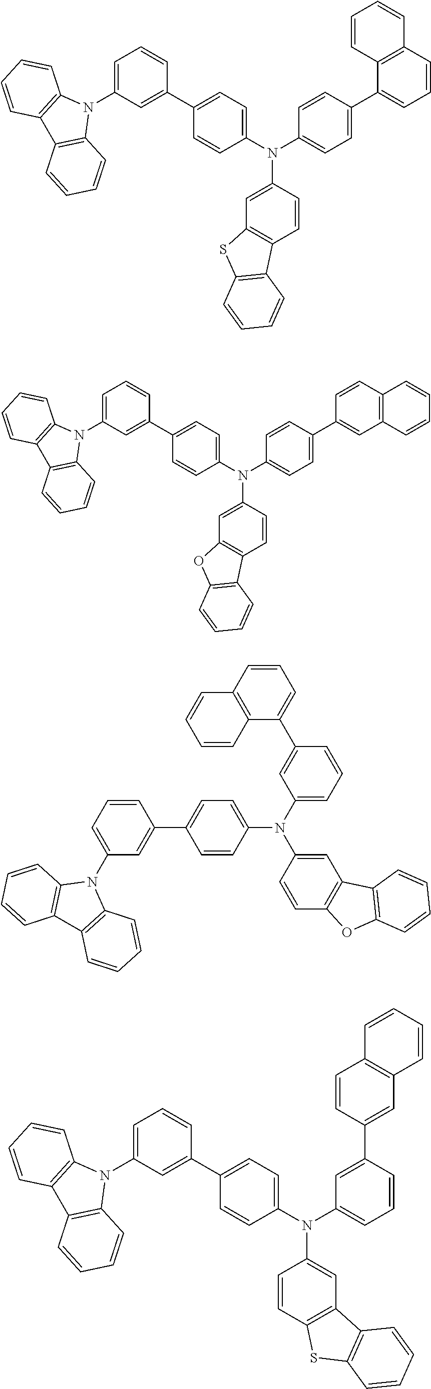

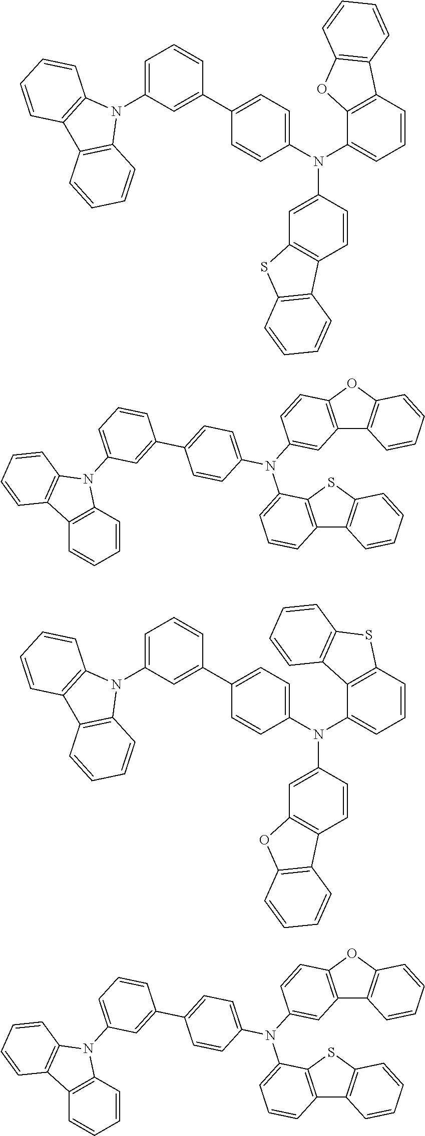

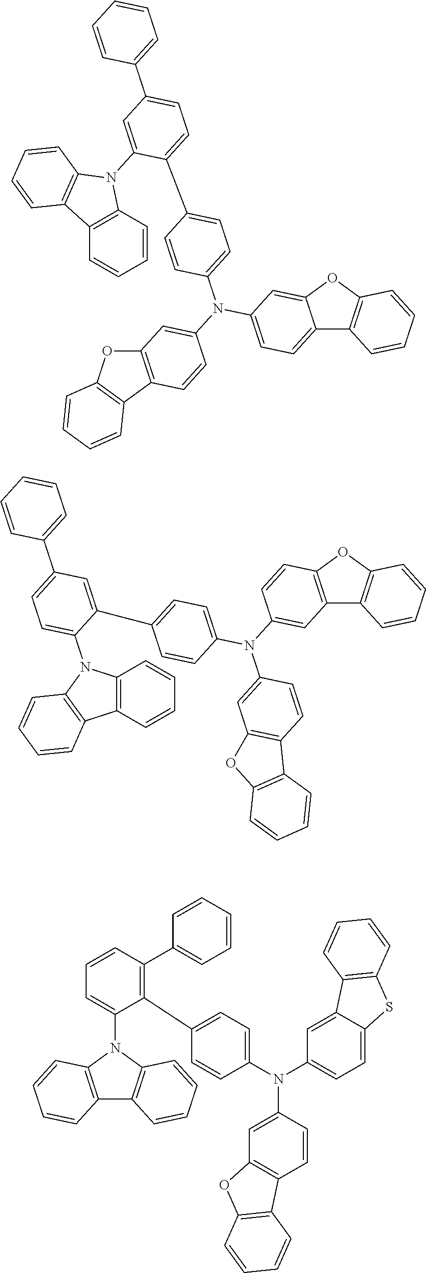 [SMILES: c1ccc(-c2ccc(-c3ccc(N(c4ccc5c(c4)oc4ccccc45)c4ccc5c(c4)oc4ccccc45)cc3)c(-n3c4ccccc4c4ccccc43)c2)cc1 ; c1ccc(-c2ccc(-n3c4ccccc4c4ccccc43)c(-c3ccc(N(c4ccc5c(c4)oc4ccccc45)c4ccc5oc6ccccc6c5c4)cc3)c2)cc1 ; c1ccc(-c2cccc(-n3c4ccccc4c4ccccc43)c2-c2ccc(N(c3ccc4c(c3)oc3ccccc34)c3ccc4sc5ccccc5c4c3)cc2)cc1]

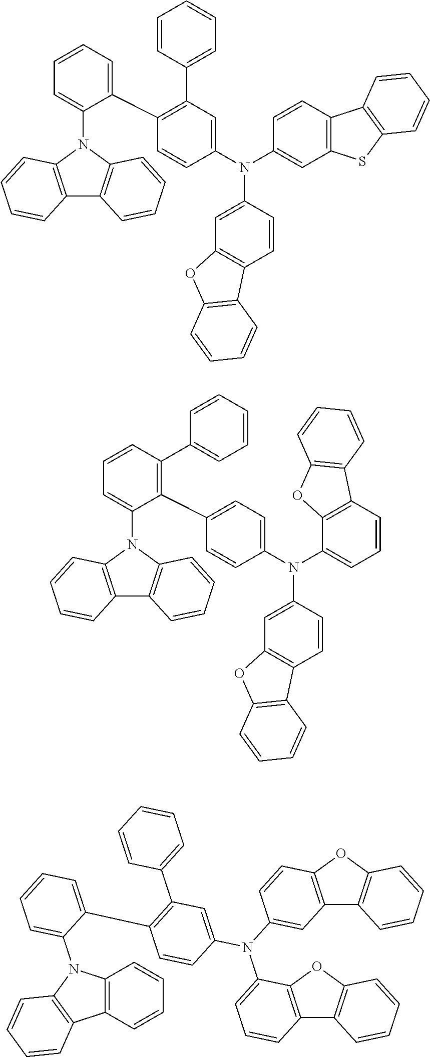

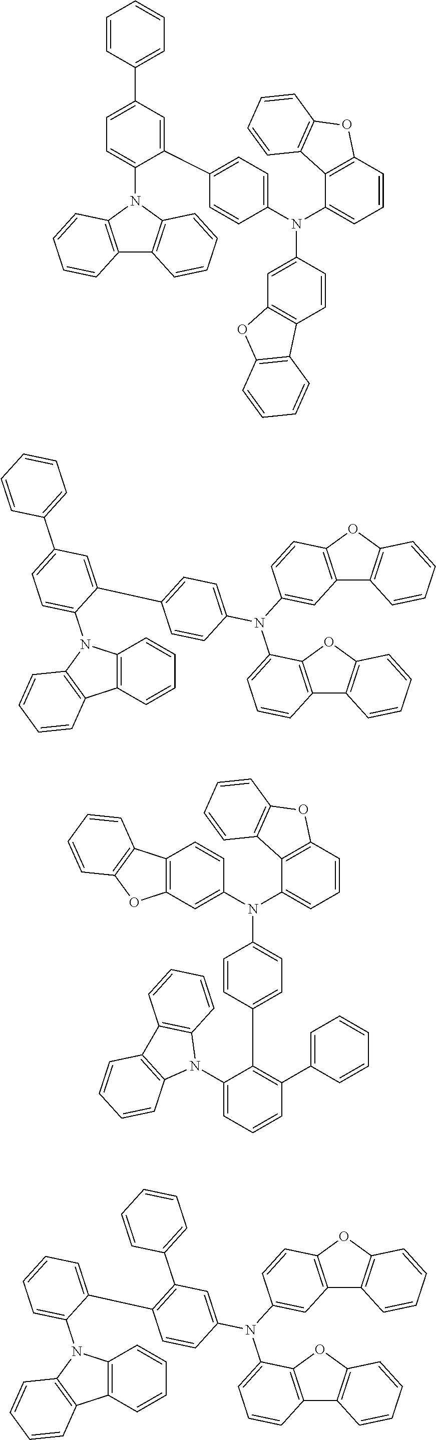 [SMILES: c1ccc(-c2ccc(-n3c4ccccc4c4ccccc43)c(-c3ccc(N(c4ccc5c(c4)oc4ccccc45)c4cccc5oc6ccccc6c45)cc3)c2)cc1 ; c1ccc(-c2ccc(-n3c4ccccc4c4ccccc43)c(-c3ccc(N(c4ccc5oc6ccccc6c5c4)c4cccc5c4oc4ccccc45)cc3)c2)cc1 ; c1ccc(-c2cccc(-n3c4ccccc4c4ccccc43)c2-c2ccc(N(c3ccc4c(c3)oc3ccccc34)c3cccc4oc5ccccc5c34)cc2)cc1 ; c1ccc(-c2cc(N(c3ccc4oc5ccccc5c4c3)c3cccc4c3oc3ccccc34)ccc2-c2ccccc2-n2c3ccccc3c3ccccc32)cc1]

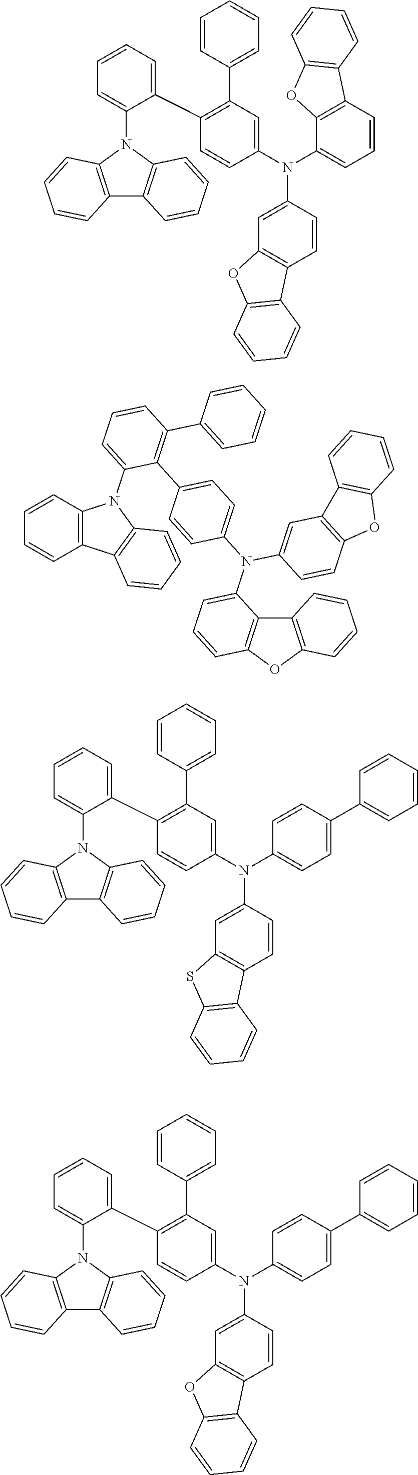

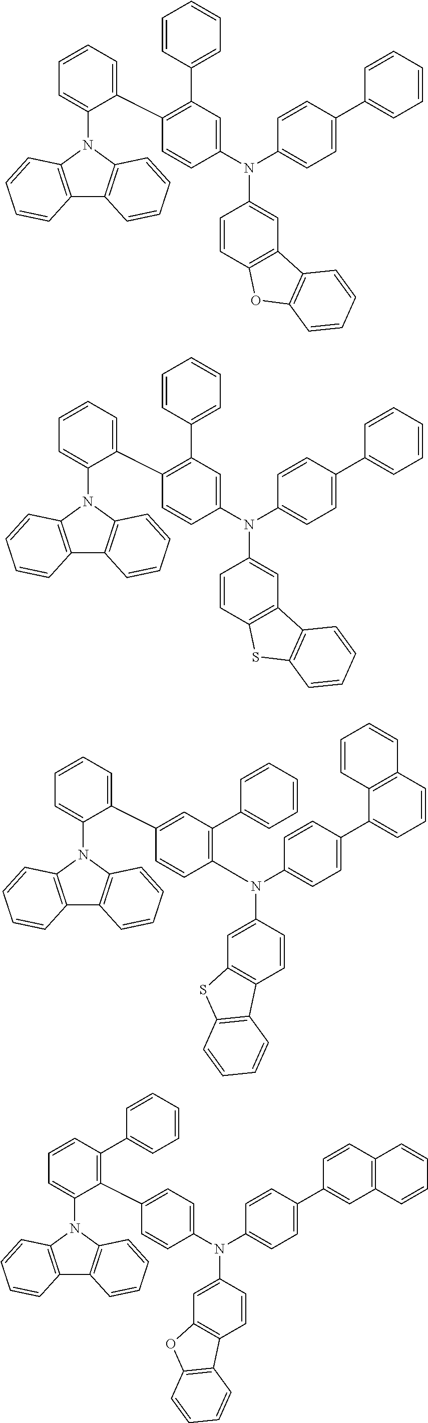 [SMILES: c1ccc(-c2ccc(N(c3ccc(-c4ccccc4-n4c5ccccc5c5ccccc54)c(-c4ccccc4)c3)c3ccc4oc5ccccc5c4c3)cc2)cc1 ; c1ccc(-c2ccc(N(c3ccc(-c4ccccc4-n4c5ccccc5c5ccccc54)c(-c4ccccc4)c3)c3ccc4sc5ccccc5c4c3)cc2)cc1 ; c1ccc(-c2cc(-c3ccccc3-n3c4ccccc4c4ccccc43)ccc2N(c2ccc(-c3cccc4ccccc34)cc2)c2ccc3c(c2)sc2ccccc23)cc1 ; c1ccc(-c2cccc(-n3c4ccccc4c4ccccc43)c2-c2ccc(N(c3ccc(-c4ccc5ccccc5c4)cc3)c3ccc4c(c3)oc3ccccc34)cc2)cc1]

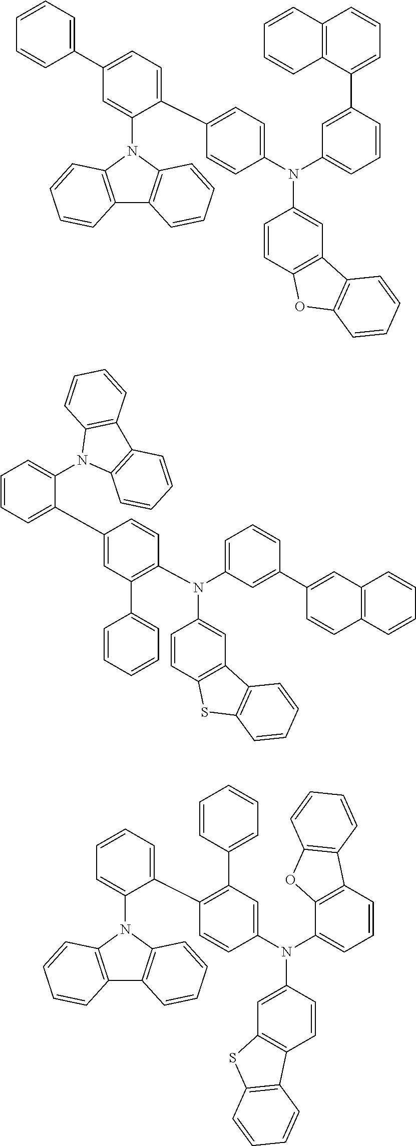 [SMILES: c1ccc(-c2ccc(-c3ccc(N(c4cccc(-c5cccc6ccccc56)c4)c4ccc5oc6ccccc6c5c4)cc3)c(-n3c4ccccc4c4ccccc43)c2)cc1 ; c1ccc(-c2cc(-c3ccccc3-n3c4ccccc4c4ccccc43)ccc2N(c2cccc(-c3ccc4ccccc4c3)c2)c2ccc3sc4ccccc4c3c2)cc1 ; c1ccc(-c2cc(N(c3ccc4c(c3)sc3ccccc34)c3cccc4c3oc3ccccc34)ccc2-c2ccccc2-n2c3ccccc3c3ccccc32)cc1]

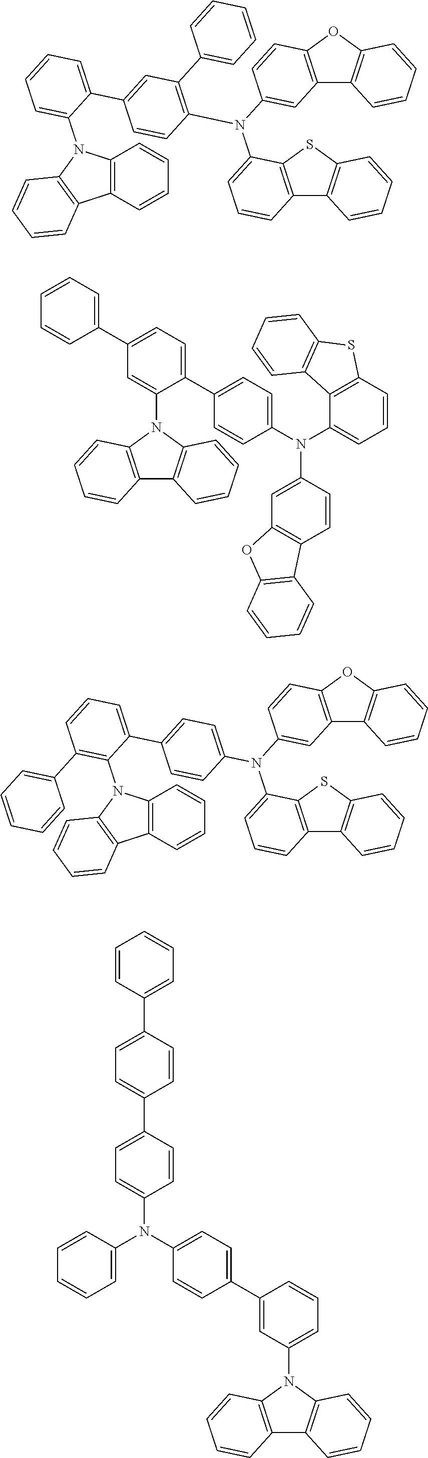

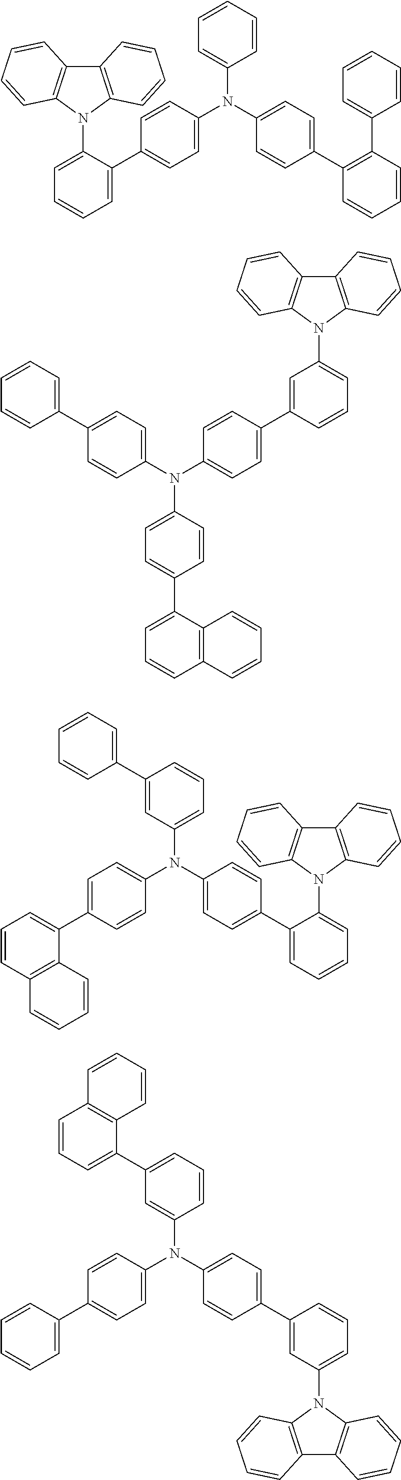

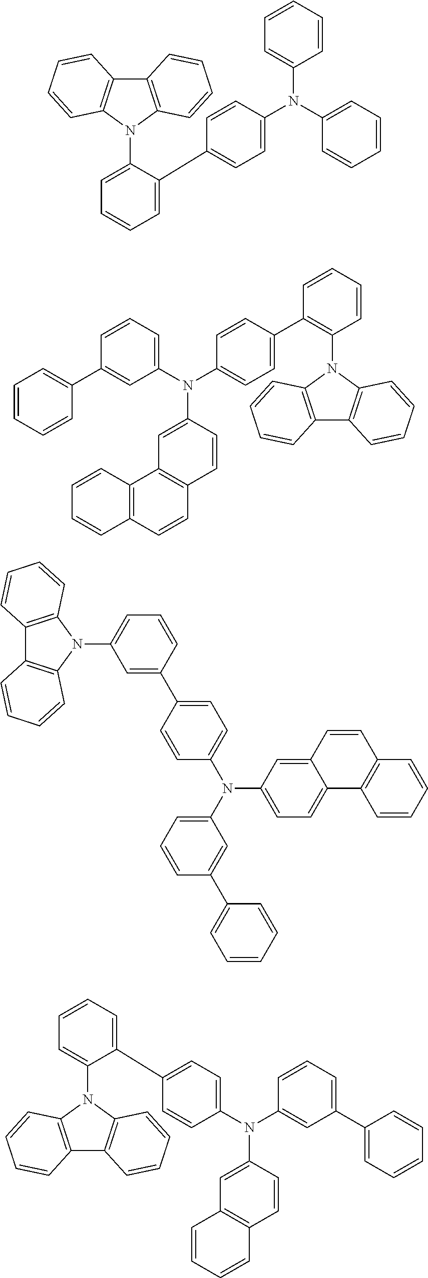 [SMILES: c1ccc(N(c2ccccc2)c2ccc(-c3ccccc3-n3c4ccccc4c4ccccc43)cc2)cc1 ; c1ccc(-c2cccc(N(c3ccc(-c4ccccc4-n4c5ccccc5c5ccccc54)cc3)c3ccc4ccc5ccccc5c4c3)c2)cc1 ; c1ccc(-c2cccc(N(c3ccc(-c4cccc(-n5c6ccccc6c6ccccc65)c4)cc3)c3ccc4c(ccc5ccccc54)c3)c2)cc1 ; c1ccc(-c2cccc(N(c3ccc(-c4ccccc4-n4c5ccccc5c5ccccc54)cc3)c3ccc4ccccc4c3)c2)cc1]

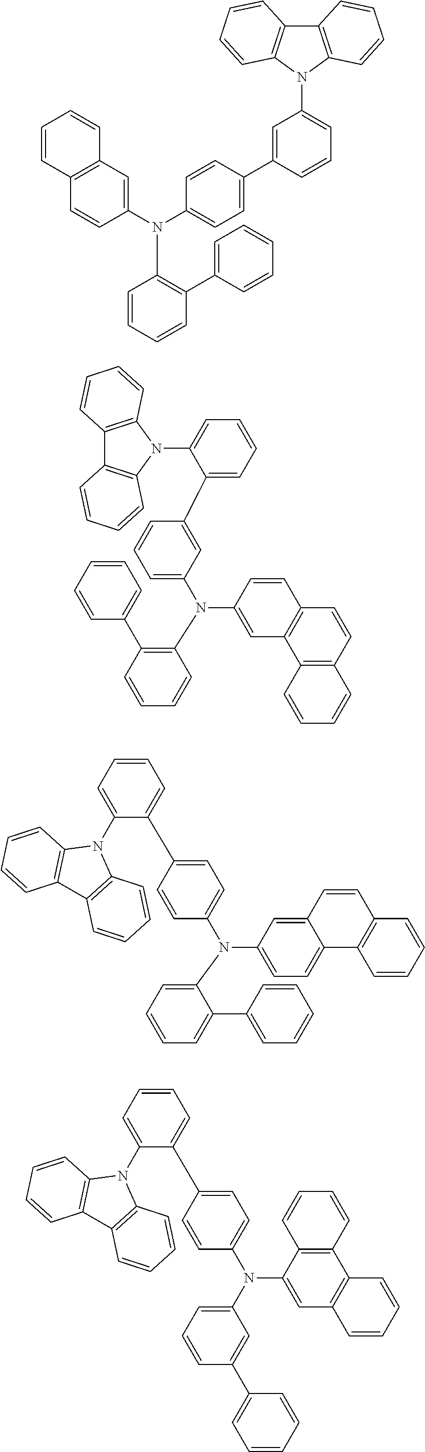

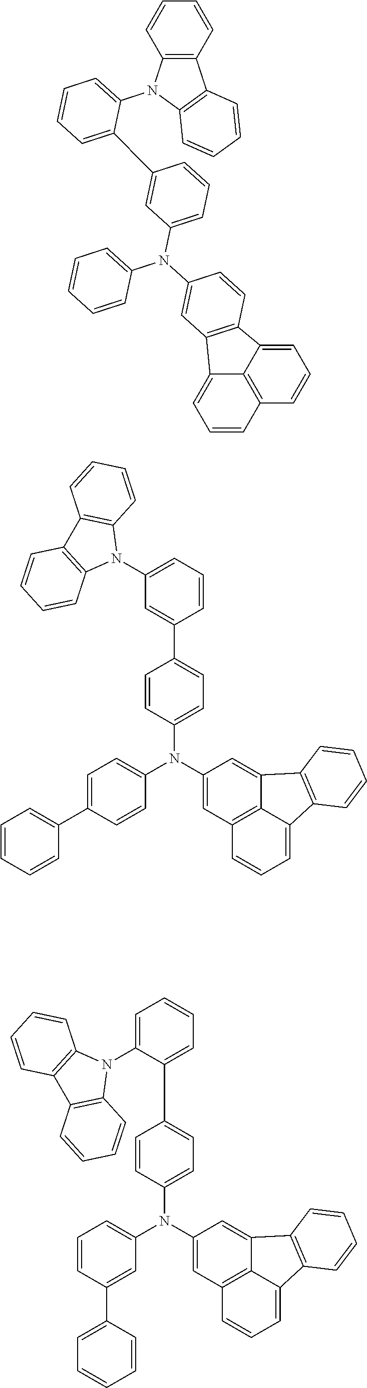 [SMILES: c1ccc(N(c2cccc(-c3ccccc3-n3c4ccccc4c4ccccc43)c2)c2ccc3c(c2)-c2cccc4cccc-3c24)cc1 ; c1ccc(-c2ccc(N(c3ccc(-c4cccc(-n5c6ccccc6c6ccccc65)c4)cc3)c3cc4c5c(cccc5c3)-c3ccccc3-4)cc2)cc1 ; c1ccc(-c2cccc(N(c3ccc(-c4ccccc4-n4c5ccccc5c5ccccc54)cc3)c3cc4c5c(cccc5c3)-c3ccccc3-4)c2)cc1]

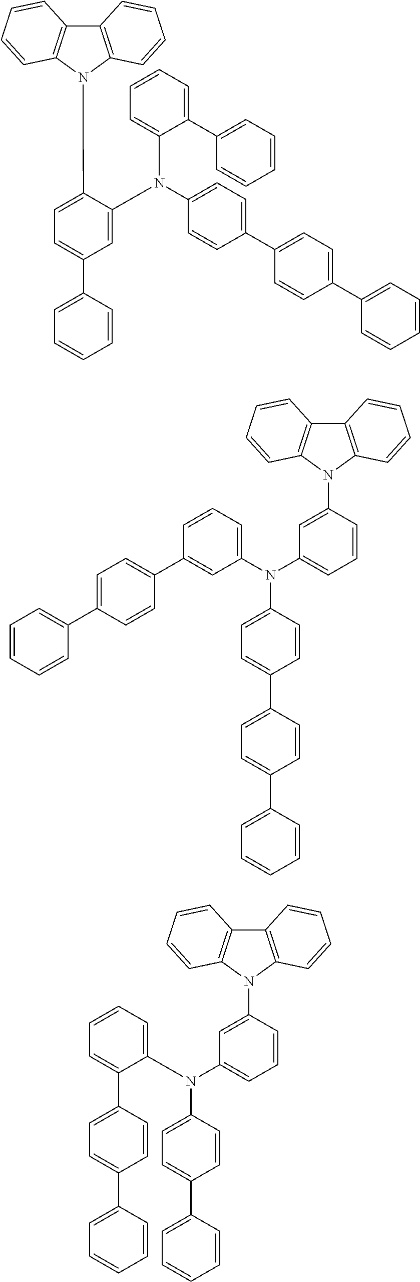

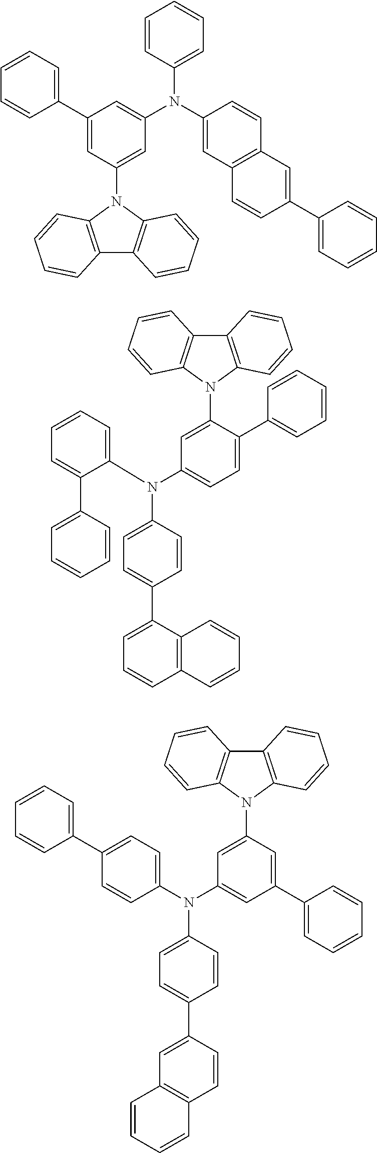

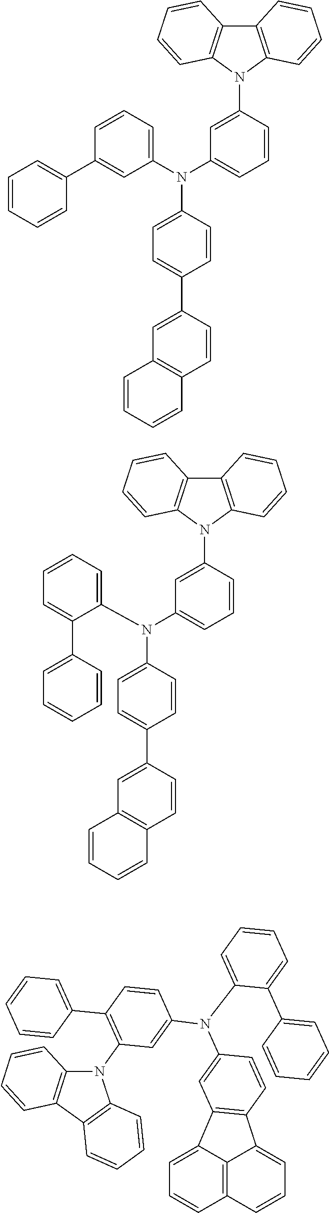

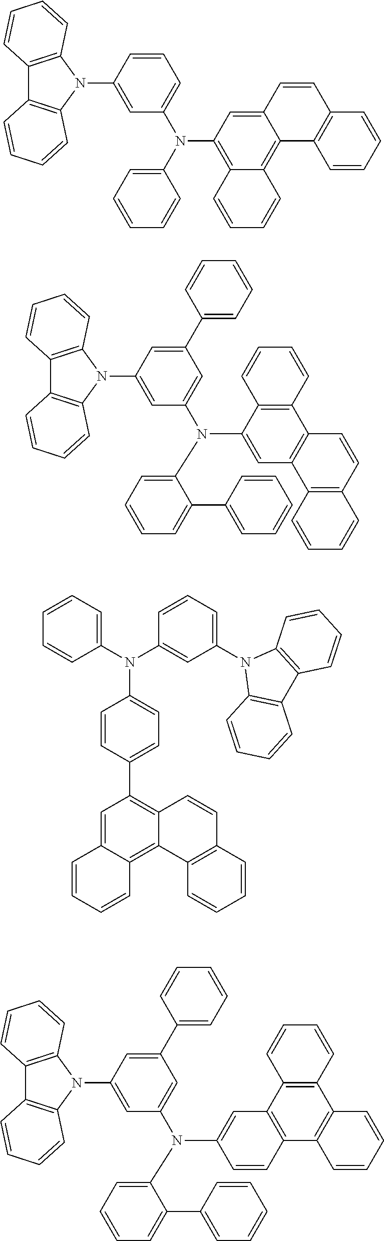

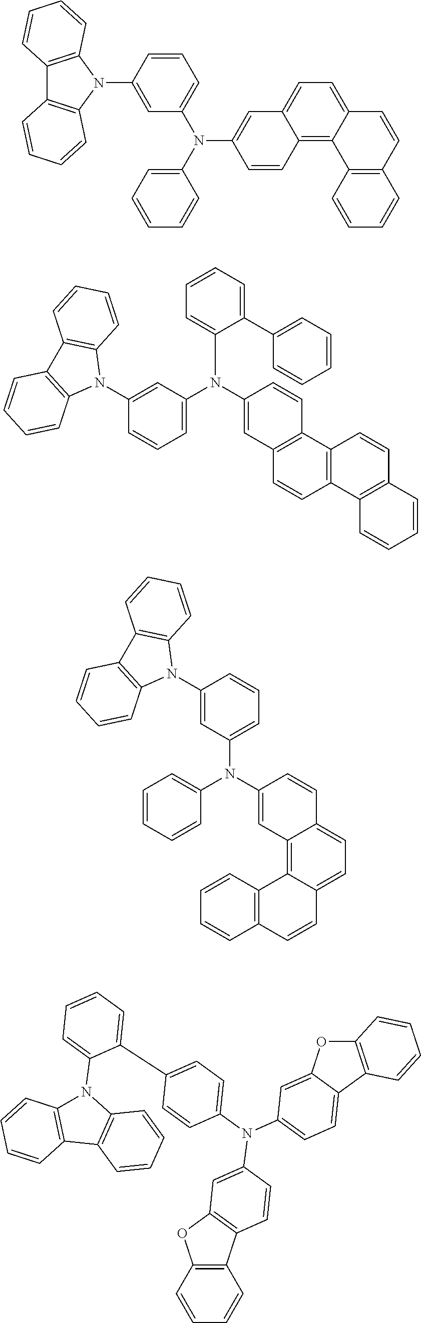 [SMILES: c1ccc(N(c2cccc(-n3c4ccccc4c4ccccc43)c2)c2ccc3c(ccc4ccc5ccccc5c43)c2)cc1 ; c1ccc(-c2ccccc2N(c2cccc(-n3c4ccccc4c4ccccc43)c2)c2ccc3c(ccc4c5ccccc5ccc34)c2)cc1 ; c1ccc(N(c2cccc(-n3c4ccccc4c4ccccc43)c2)c2ccc3ccc4ccc5ccccc5c4c3c2)cc1 ; c1ccc(-n2c3ccccc3c3ccccc32)c(-c2ccc(N(c3ccc4c(c3)oc3ccccc34)c3ccc4c(c3)oc3ccccc34)cc2)c1]

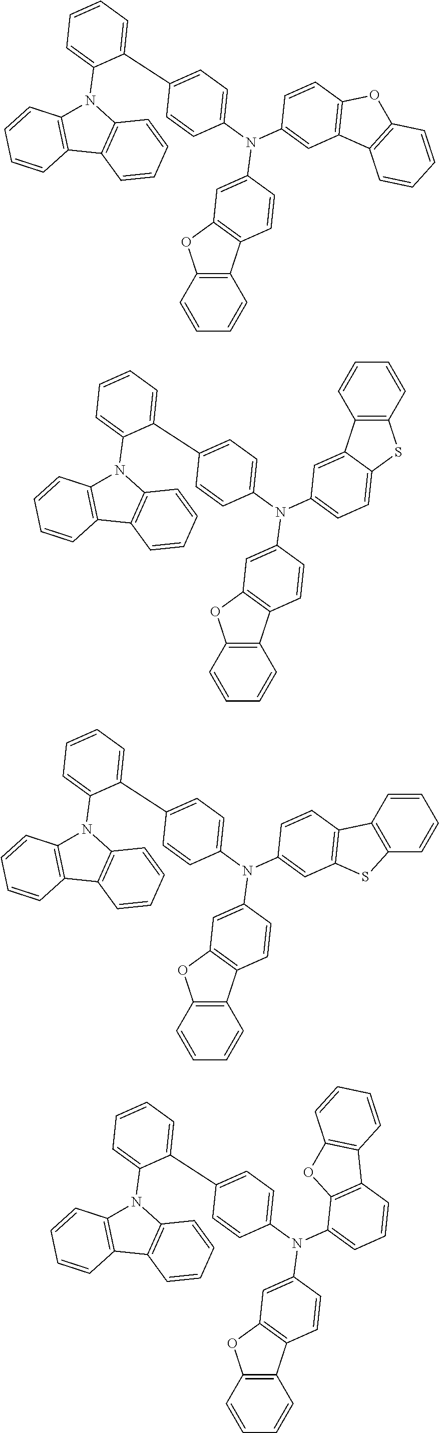 [SMILES: c1ccc(-n2c3ccccc3c3ccccc32)c(-c2ccc(N(c3ccc4c(c3)oc3ccccc34)c3ccc4oc5ccccc5c4c3)cc2)c1 ; c1ccc(-n2c3ccccc3c3ccccc32)c(-c2ccc(N(c3ccc4c(c3)oc3ccccc34)c3ccc4sc5ccccc5c4c3)cc2)c1 ; c1ccc(-n2c3ccccc3c3ccccc32)c(-c2ccc(N(c3ccc4c(c3)oc3ccccc34)c3ccc4c(c3)sc3ccccc34)cc2)c1 ; c1ccc(-n2c3ccccc3c3ccccc32)c(-c2ccc(N(c3ccc4c(c3)oc3ccccc34)c3cccc4c3oc3ccccc34)cc2)c1]

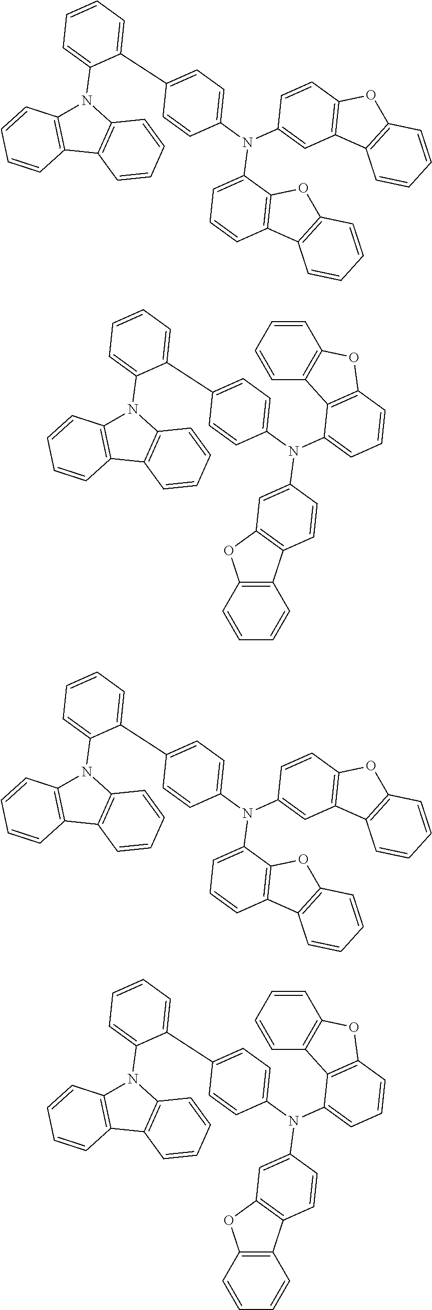 [SMILES: c1ccc(-n2c3ccccc3c3ccccc32)c(-c2ccc(N(c3ccc4oc5ccccc5c4c3)c3cccc4c3oc3ccccc34)cc2)c1 ; c1ccc(-n2c3ccccc3c3ccccc32)c(-c2ccc(N(c3ccc4c(c3)oc3ccccc34)c3cccc4oc5ccccc5c34)cc2)c1 ; c1ccc(-n2c3ccccc3c3ccccc32)c(-c2ccc(N(c3ccc4oc5ccccc5c4c3)c3cccc4c3oc3ccccc34)cc2)c1 ; c1ccc(-n2c3ccccc3c3ccccc32)c(-c2ccc(N(c3ccc4c(c3)oc3ccccc34)c3cccc4oc5ccccc5c34)cc2)c1]

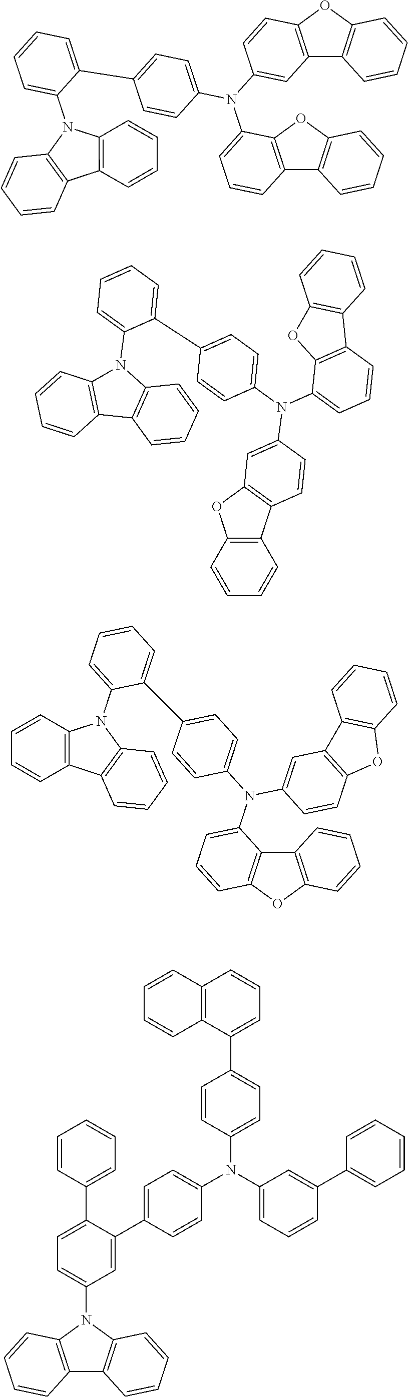 [SMILES: c1ccc(-n2c3ccccc3c3ccccc32)c(-c2ccc(N(c3ccc4oc5ccccc5c4c3)c3cccc4c3oc3ccccc34)cc2)c1 ; c1ccc(-n2c3ccccc3c3ccccc32)c(-c2ccc(N(c3ccc4c(c3)oc3ccccc34)c3cccc4c3oc3ccccc34)cc2)c1 ; c1ccc(-n2c3ccccc3c3ccccc32)c(-c2ccc(N(c3ccc4oc5ccccc5c4c3)c3cccc4oc5ccccc5c34)cc2)c1 ; c1ccc(-c2cccc(N(c3ccc(-c4cc(-n5c6ccccc6c6ccccc65)ccc4-c4ccccc4)cc3)c3ccc(-c4cccc5ccccc45)cc3)c2)cc1]

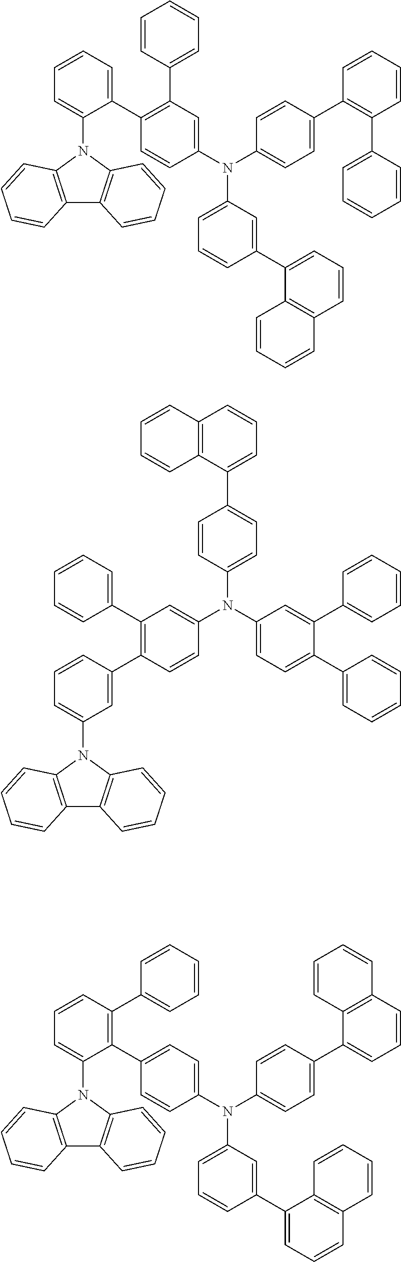 [SMILES: c1ccc(-c2ccccc2-c2ccc(N(c3cccc(-c4cccc5ccccc45)c3)c3ccc(-c4ccccc4-n4c5ccccc5c5ccccc54)c(-c4ccccc4)c3)cc2)cc1 ; c1ccc(-c2ccc(N(c3ccc(-c4cccc5ccccc45)cc3)c3ccc(-c4cccc(-n5c6ccccc6c6ccccc65)c4)c(-c4ccccc4)c3)cc2-c2ccccc2)cc1 ; c1ccc(-c2cccc(-n3c4ccccc4c4ccccc43)c2-c2ccc(N(c3ccc(-c4cccc5ccccc45)cc3)c3cccc(-c4cccc5ccccc45)c3)cc2)cc1]

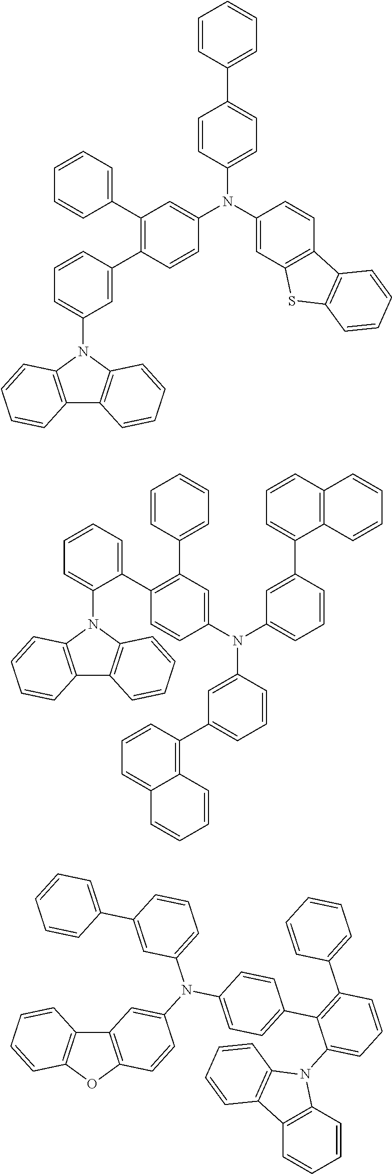 [SMILES: c1ccc(-c2ccc(N(c3ccc(-c4cccc(-n5c6ccccc6c6ccccc65)c4)c(-c4ccccc4)c3)c3ccc4c(c3)sc3ccccc34)cc2)cc1 ; c1ccc(-c2cc(N(c3cccc(-c4cccc5ccccc45)c3)c3cccc(-c4cccc5ccccc45)c3)ccc2-c2ccccc2-n2c3ccccc3c3ccccc32)cc1 ; c1ccc(-c2cccc(N(c3ccc(-c4c(-c5ccccc5)cccc4-n4c5ccccc5c5ccccc54)cc3)c3ccc4oc5ccccc5c4c3)c2)cc1]

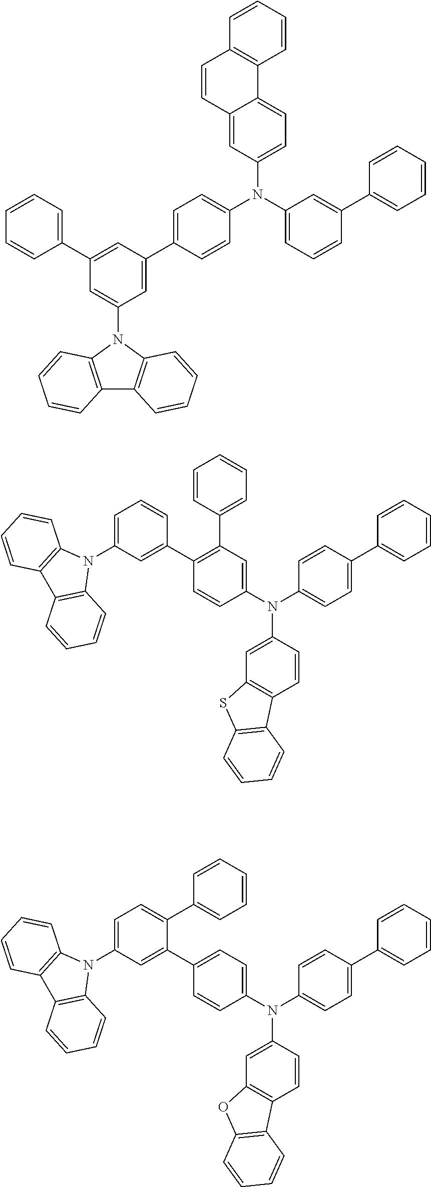 [SMILES: c1ccc(-c2cccc(N(c3ccc(-c4cc(-c5ccccc5)cc(-n5c6ccccc6c6ccccc65)c4)cc3)c3ccc4c(ccc5ccccc54)c3)c2)cc1 ; c1ccc(-c2ccc(N(c3ccc(-c4cccc(-n5c6ccccc6c6ccccc65)c4)c(-c4ccccc4)c3)c3ccc4c(c3)sc3ccccc34)cc2)cc1 ; c1ccc(-c2ccc(N(c3ccc(-c4cc(-n5c6ccccc6c6ccccc65)ccc4-c4ccccc4)cc3)c3ccc4c(c3)oc3ccccc34)cc2)cc1]

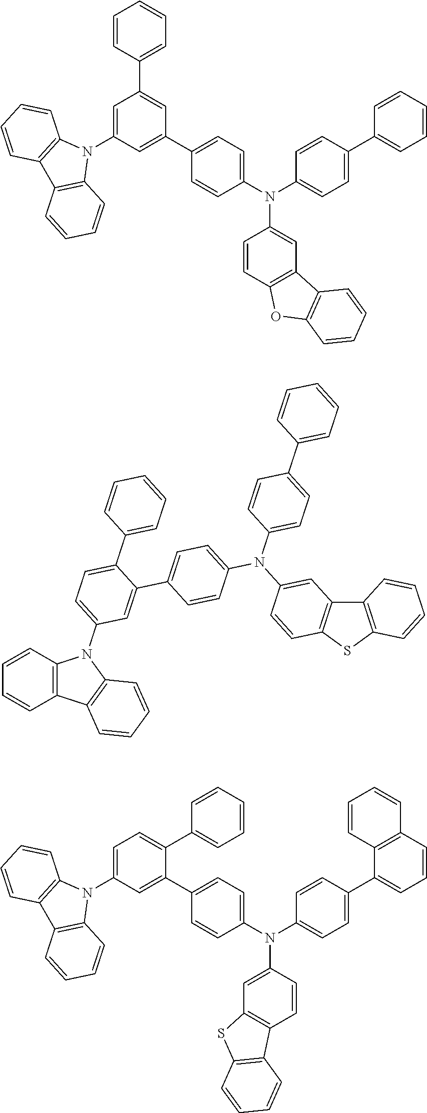

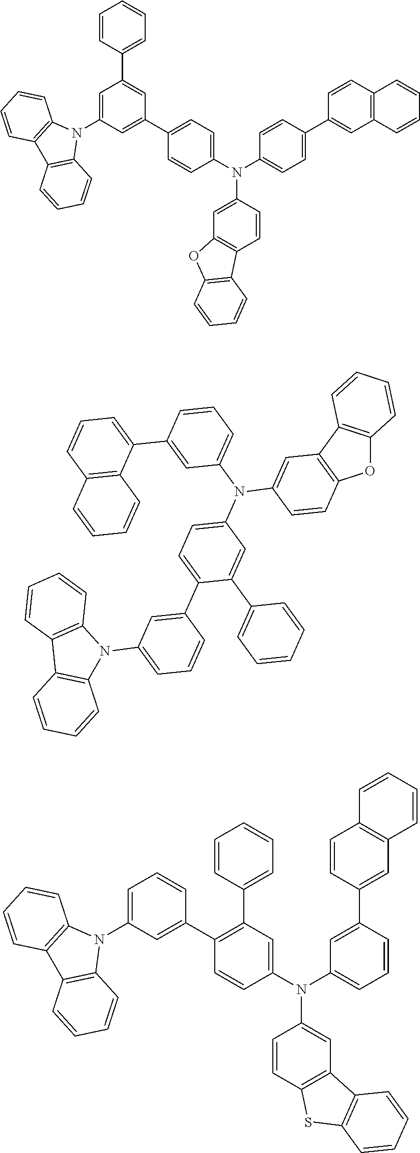 [SMILES: c1ccc(-c2cc(-c3ccc(N(c4ccc(-c5ccc6ccccc6c5)cc4)c4ccc5c(c4)oc4ccccc45)cc3)cc(-n3c4ccccc4c4ccccc43)c2)cc1 ; c1ccc(-c2cc(N(c3cccc(-c4cccc5ccccc45)c3)c3ccc4oc5ccccc5c4c3)ccc2-c2cccc(-n3c4ccccc4c4ccccc43)c2)cc1 ; c1ccc(-c2cc(N(c3cccc(-c4ccc5ccccc5c4)c3)c3ccc4sc5ccccc5c4c3)ccc2-c2cccc(-n3c4ccccc4c4ccccc43)c2)cc1]

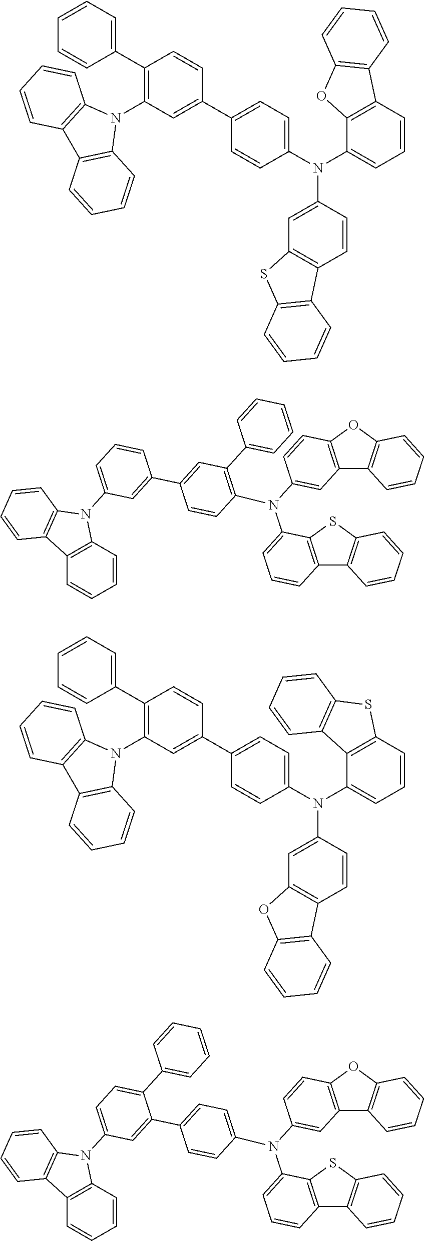

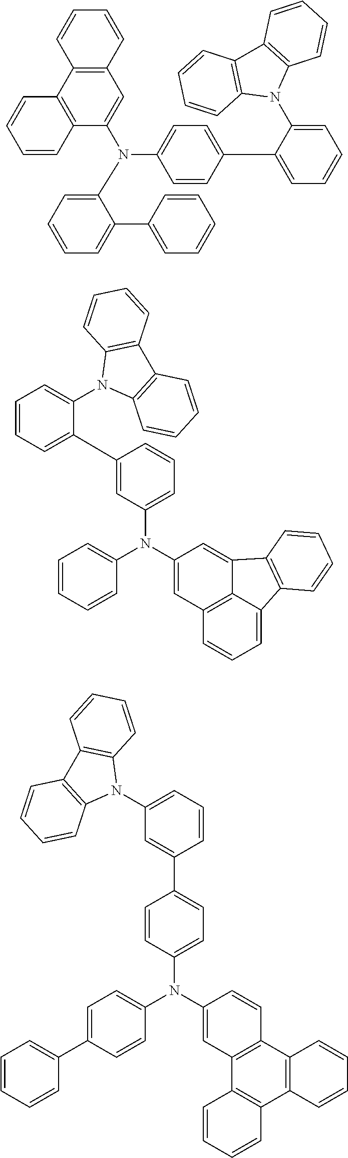

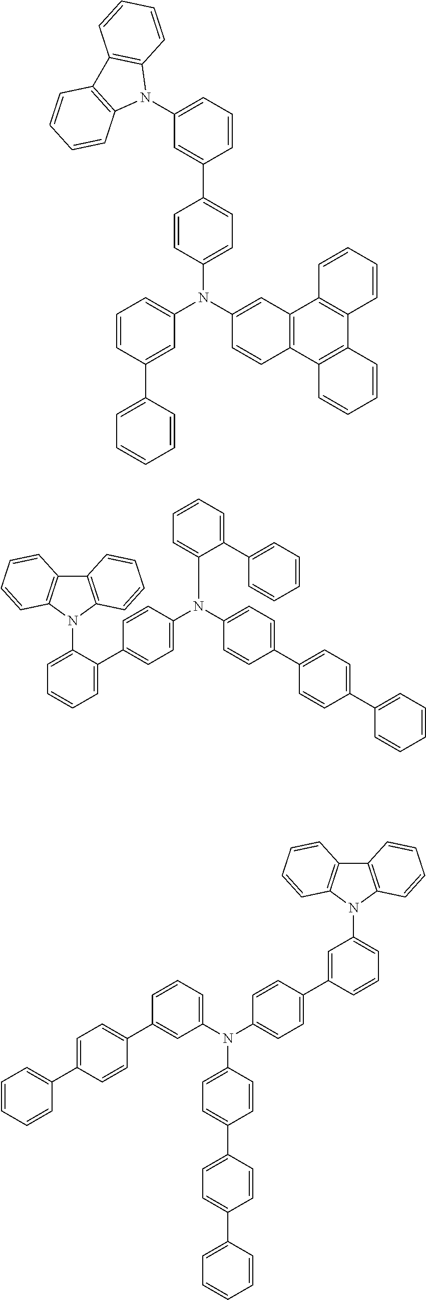 [SMILES: c1ccc(-c2cccc(N(c3ccc(-c4cccc(-n5c6ccccc6c6ccccc65)c4)cc3)c3ccc4c5ccccc5c5ccccc5c4c3)c2)cc1 ; c1ccc(-c2ccc(-c3ccc(N(c4ccc(-c5ccccc5-n5c6ccccc6c6ccccc65)cc4)c4ccccc4-c4ccccc4)cc3)cc2)cc1 ; c1ccc(-c2ccc(-c3ccc(N(c4ccc(-c5cccc(-n6c7ccccc7c7ccccc76)c5)cc4)c4cccc(-c5ccc(-c6ccccc6)cc5)c4)cc3)cc2)cc1]

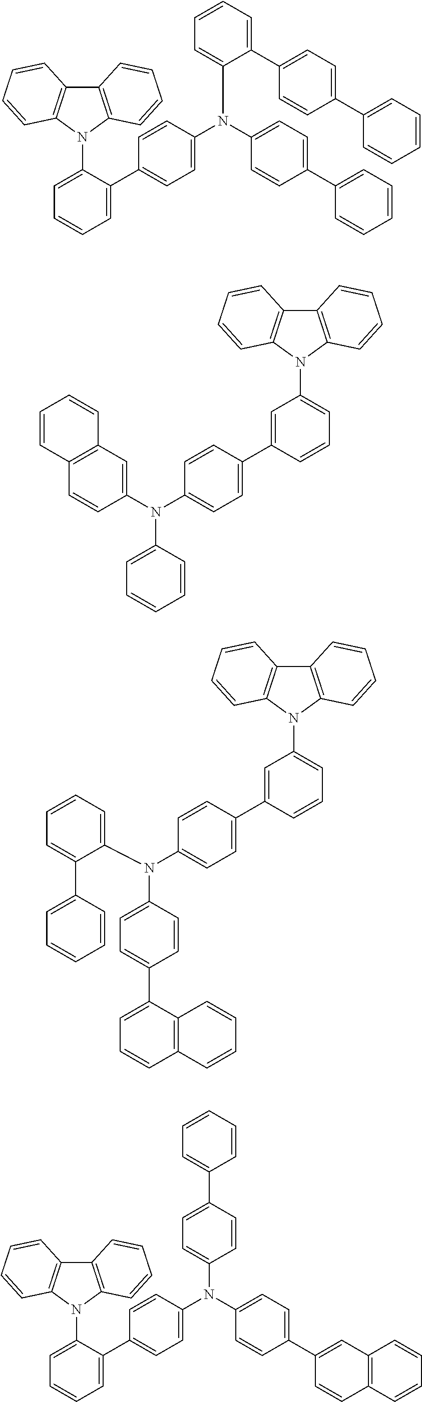

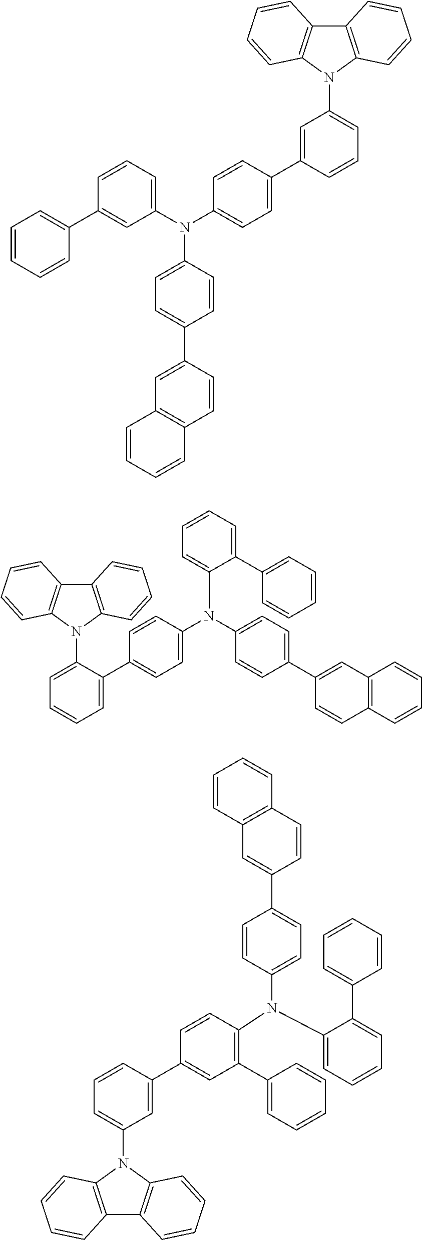 [SMILES: c1ccc(-c2cccc(N(c3ccc(-c4cccc(-n5c6ccccc6c6ccccc65)c4)cc3)c3ccc(-c4ccc5ccccc5c4)cc3)c2)cc1 ; c1ccc(-c2ccccc2N(c2ccc(-c3ccc4ccccc4c3)cc2)c2ccc(-c3ccccc3-n3c4ccccc4c4ccccc43)cc2)cc1 ; c1ccc(-c2ccccc2N(c2ccc(-c3ccc4ccccc4c3)cc2)c2ccc(-c3cccc(-n4c5ccccc5c5ccccc54)c3)cc2-c2ccccc2)cc1]

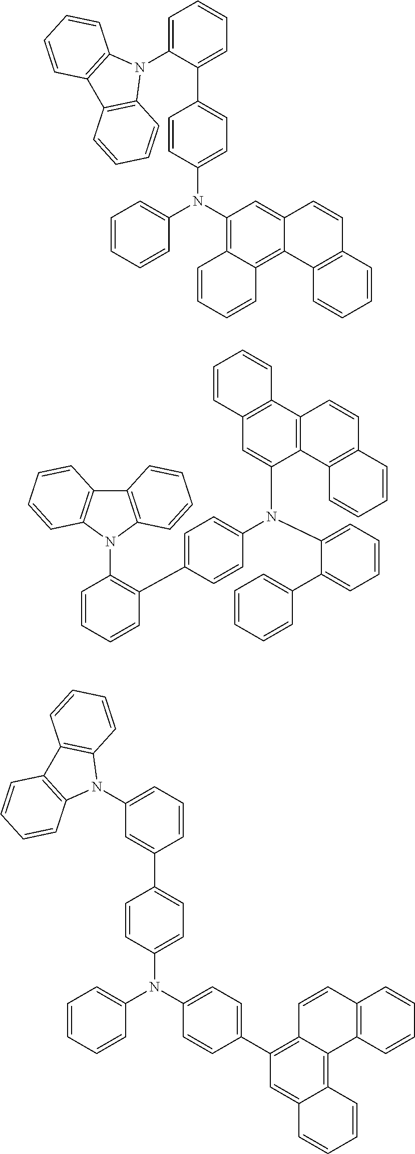

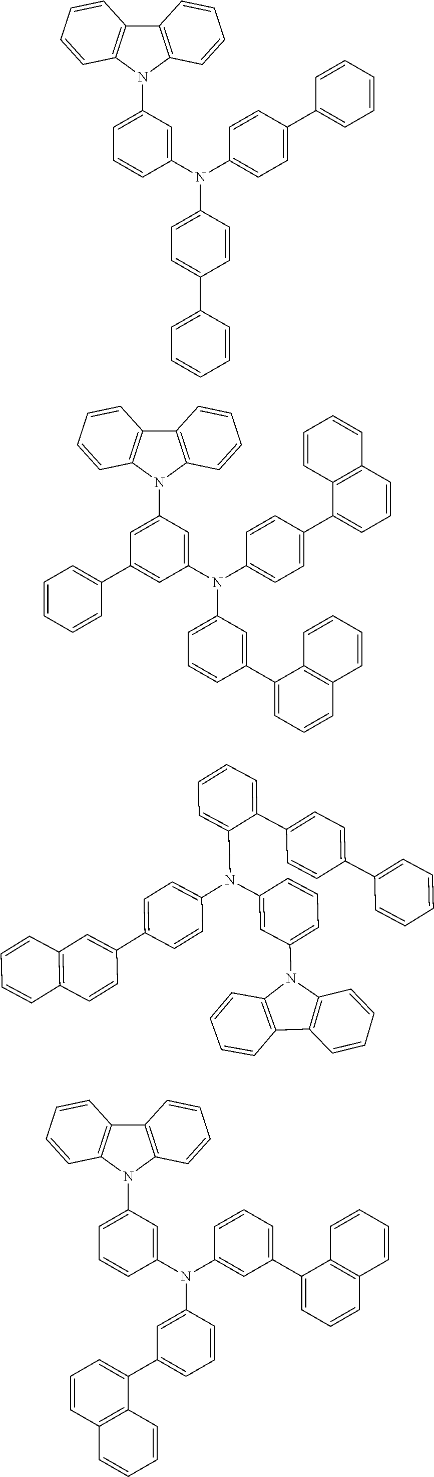

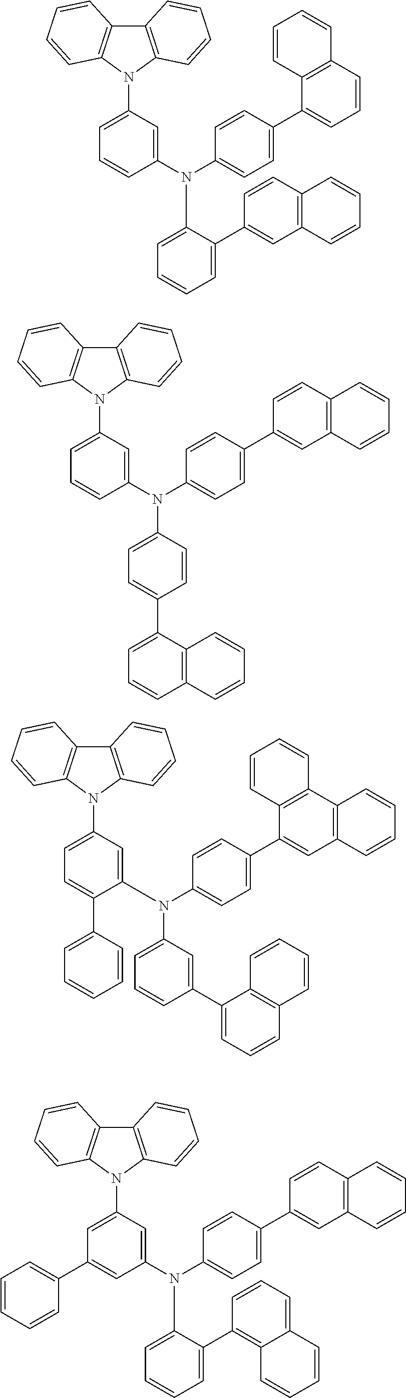

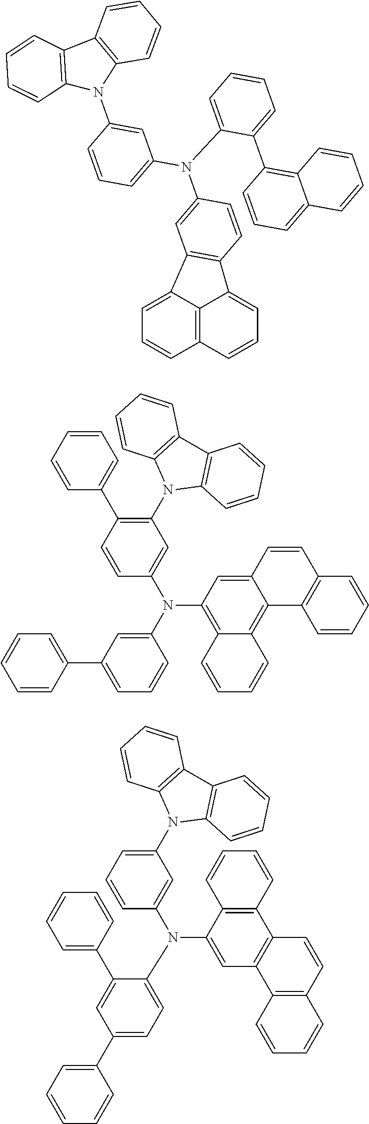

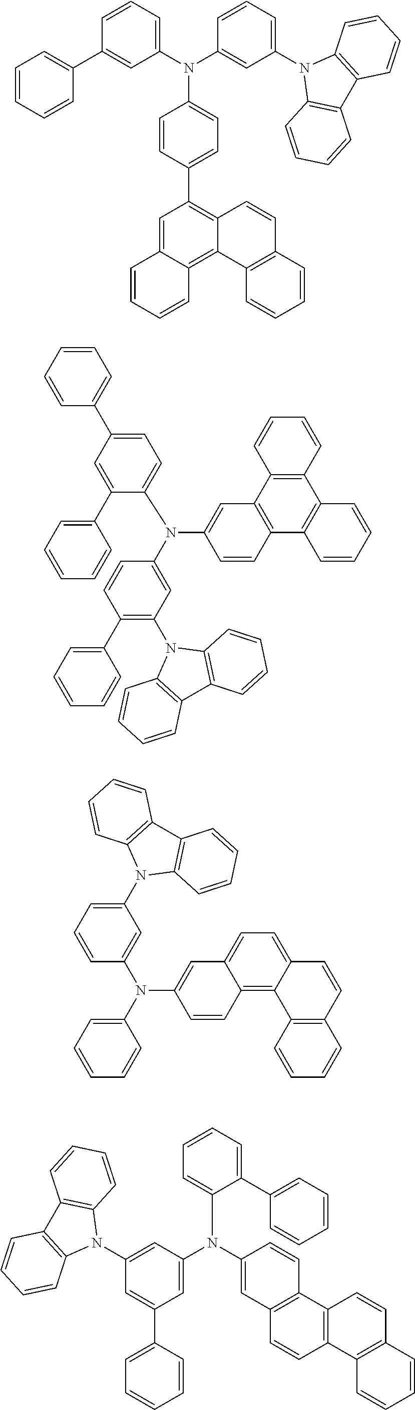

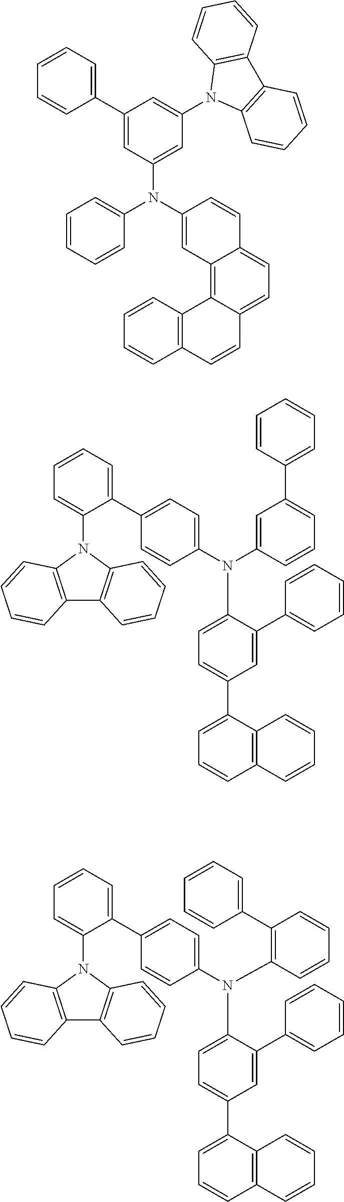 [SMILES: c1ccc(-c2cc(N(c3ccccc3)c3ccc4ccc5ccc6ccccc6c5c4c3)cc(-n3c4ccccc4c4ccccc43)c2)cc1 ; c1ccc(-c2cccc(N(c3ccc(-c4ccccc4-n4c5ccccc5c5ccccc54)cc3)c3ccc(-c4cccc5ccccc45)cc3-c3ccccc3)c2)cc1 ; c1ccc(-c2ccccc2N(c2ccc(-c3ccccc3-n3c4ccccc4c4ccccc43)cc2)c2ccc(-c3cccc4ccccc34)cc2-c2ccccc2)cc1]

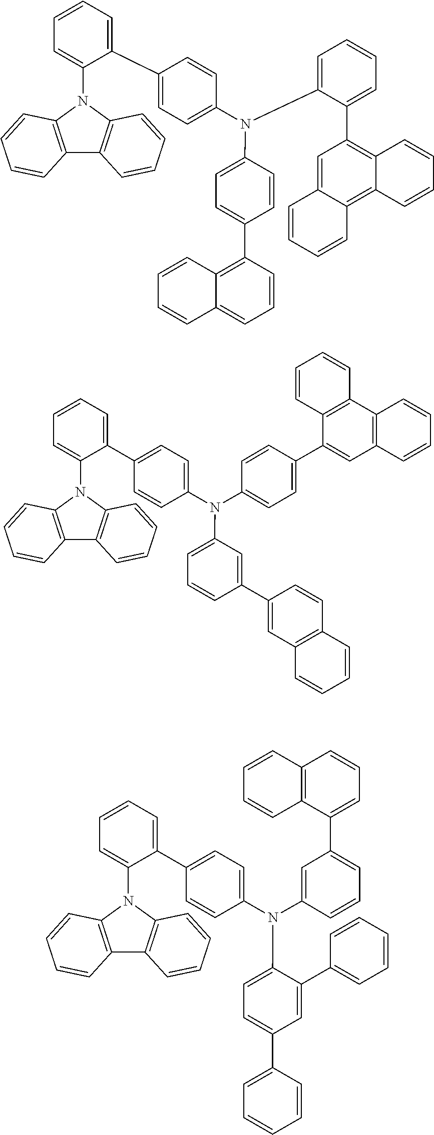 [SMILES: c1ccc(N(c2ccc(-c3ccccc3-n3c4ccccc4c4ccccc43)cc2)c2ccc(-c3cccc4ccccc34)cc2)c(-c2cc3ccccc3c3ccccc23)c1 ; c1cc(-c2ccc3ccccc3c2)cc(N(c2ccc(-c3ccccc3-n3c4ccccc4c4ccccc43)cc2)c2ccc(-c3cc4ccccc4c4ccccc34)cc2)c1 ; c1ccc(-c2ccc(N(c3ccc(-c4ccccc4-n4c5ccccc5c5ccccc54)cc3)c3cccc(-c4cccc5ccccc45)c3)c(-c3ccccc3)c2)cc1]

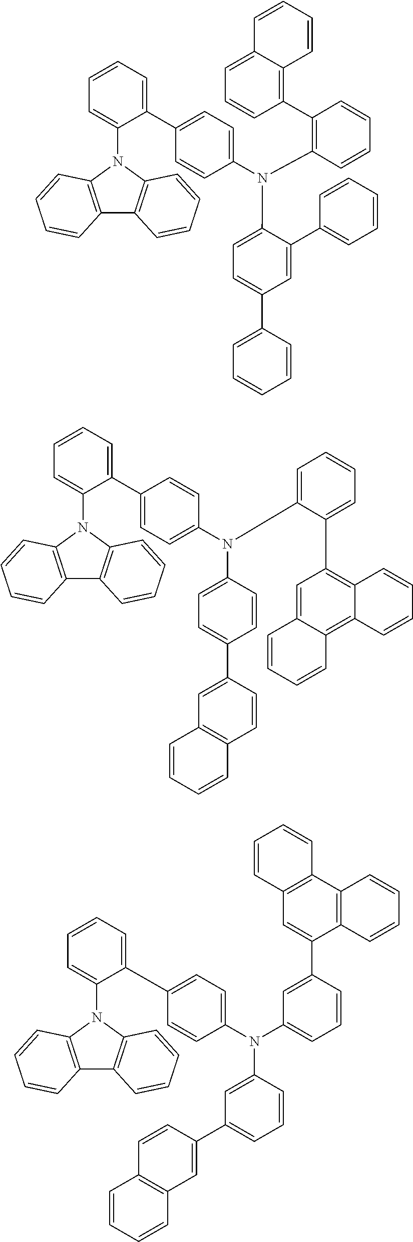 [SMILES: c1ccc(-c2ccc(N(c3ccc(-c4ccccc4-n4c5ccccc5c5ccccc54)cc3)c3ccccc3-c3cccc4ccccc34)c(-c3ccccc3)c2)cc1 ; c1ccc(N(c2ccc(-c3ccc4ccccc4c3)cc2)c2ccc(-c3ccccc3-n3c4ccccc4c4ccccc43)cc2)c(-c2cc3ccccc3c3ccccc23)c1 ; c1cc(-c2ccc3ccccc3c2)cc(N(c2ccc(-c3ccccc3-n3c4ccccc4c4ccccc43)cc2)c2cccc(-c3cc4ccccc4c4ccccc34)c2)c1]

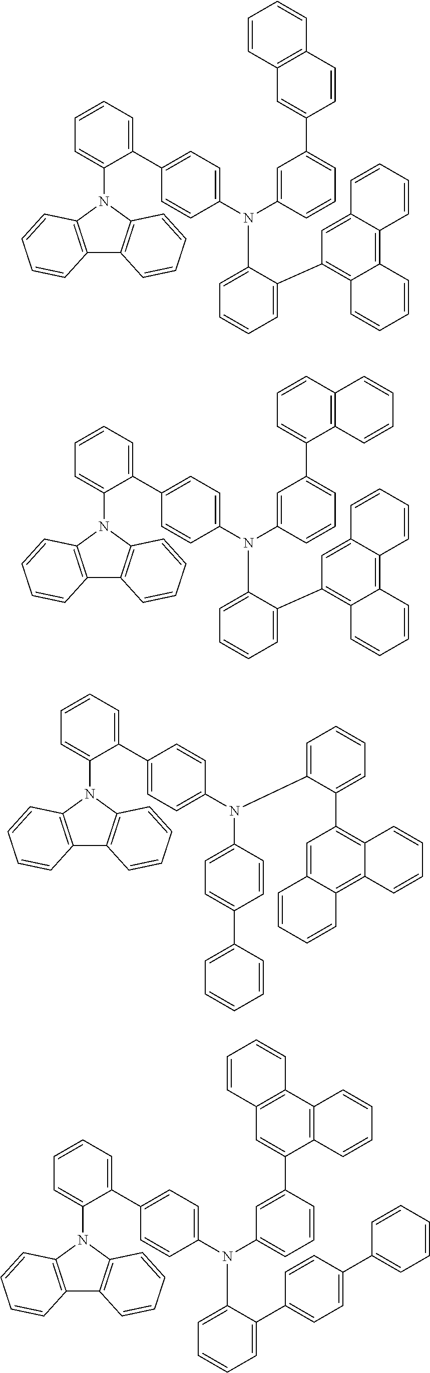

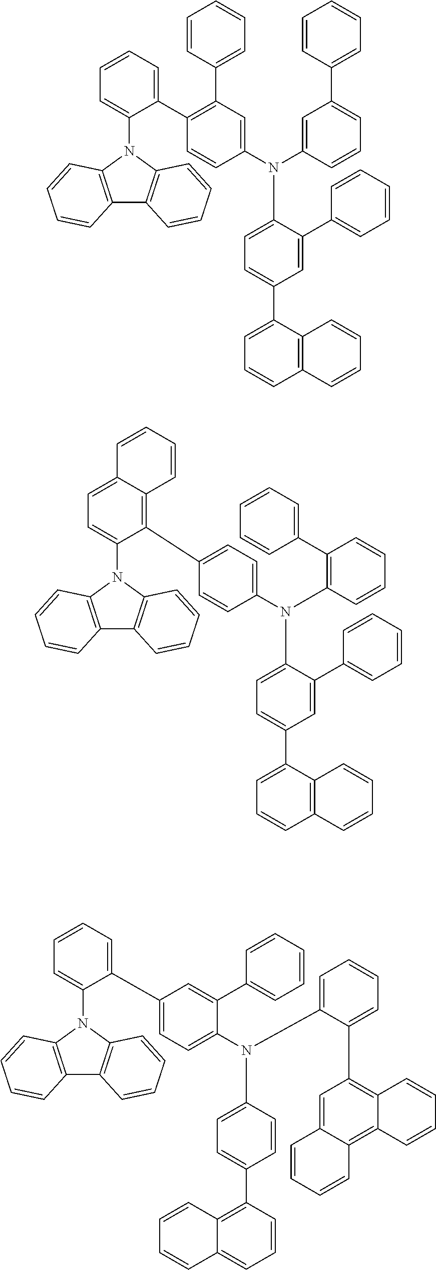 [SMILES: c1ccc(-c2cccc(N(c3ccc(-c4ccccc4-n4c5ccccc5c5ccccc54)c(-c4ccccc4)c3)c3ccc(-c4cccc5ccccc45)cc3-c3ccccc3)c2)cc1 ; c1ccc(-c2ccccc2N(c2ccc(-c3c(-n4c5ccccc5c5ccccc54)ccc4ccccc34)cc2)c2ccc(-c3cccc4ccccc34)cc2-c2ccccc2)cc1 ; c1ccc(-c2cc(-c3ccccc3-n3c4ccccc4c4ccccc43)ccc2N(c2ccc(-c3cccc4ccccc34)cc2)c2ccccc2-c2cc3ccccc3c3ccccc23)cc1]

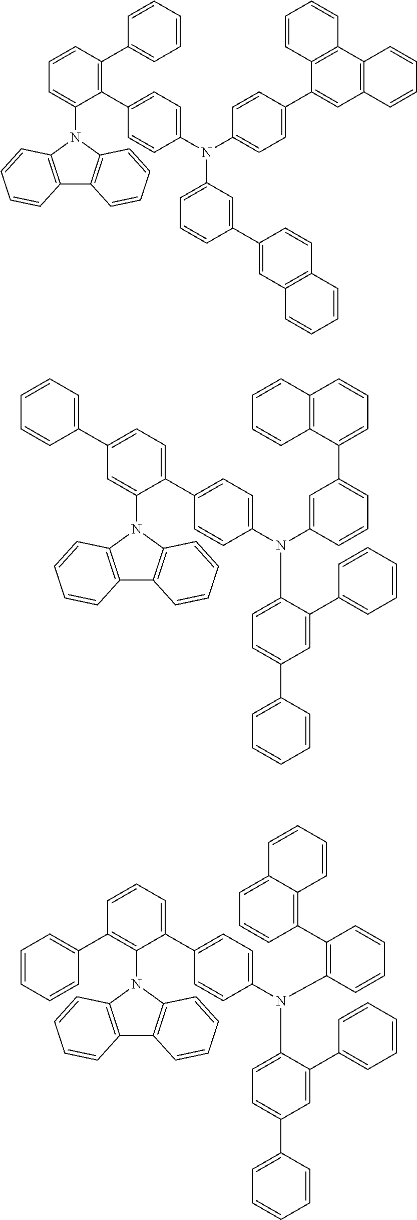 [SMILES: c1ccc(-c2cccc(-n3c4ccccc4c4ccccc43)c2-c2ccc(N(c3ccc(-c4cc5ccccc5c5ccccc45)cc3)c3cccc(-c4ccc5ccccc5c4)c3)cc2)cc1 ; c1ccc(-c2ccc(N(c3ccc(-c4ccc(-c5ccccc5)cc4-n4c5ccccc5c5ccccc54)cc3)c3cccc(-c4cccc5ccccc45)c3)c(-c3ccccc3)c2)cc1 ; c1ccc(-c2ccc(N(c3ccc(-c4cccc(-c5ccccc5)c4-n4c5ccccc5c5ccccc54)cc3)c3ccccc3-c3cccc4ccccc34)c(-c3ccccc3)c2)cc1]

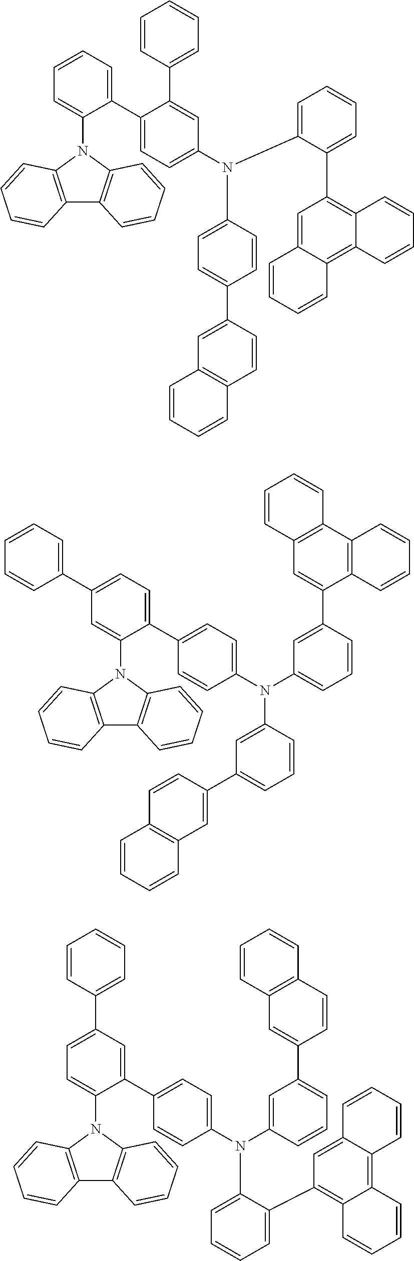 [SMILES: c1ccc(-c2cc(N(c3ccc(-c4ccc5ccccc5c4)cc3)c3ccccc3-c3cc4ccccc4c4ccccc34)ccc2-c2ccccc2-n2c3ccccc3c3ccccc32)cc1 ; c1ccc(-c2ccc(-c3ccc(N(c4cccc(-c5ccc6ccccc6c5)c4)c4cccc(-c5cc6ccccc6c6ccccc56)c4)cc3)c(-n3c4ccccc4c4ccccc43)c2)cc1 ; c1ccc(-c2ccc(-n3c4ccccc4c4ccccc43)c(-c3ccc(N(c4cccc(-c5ccc6ccccc6c5)c4)c4ccccc4-c4cc5ccccc5c5ccccc45)cc3)c2)cc1]

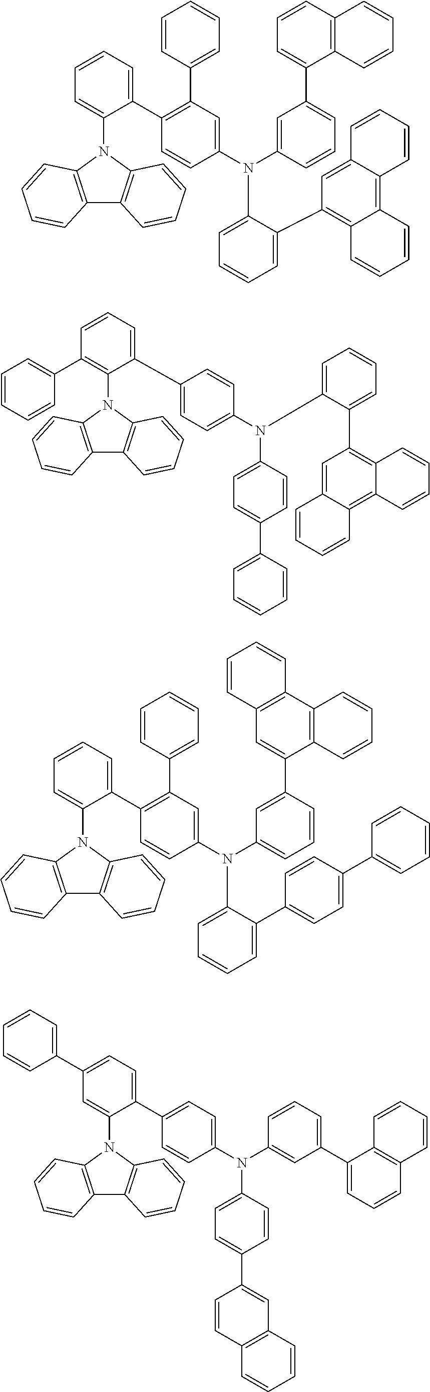

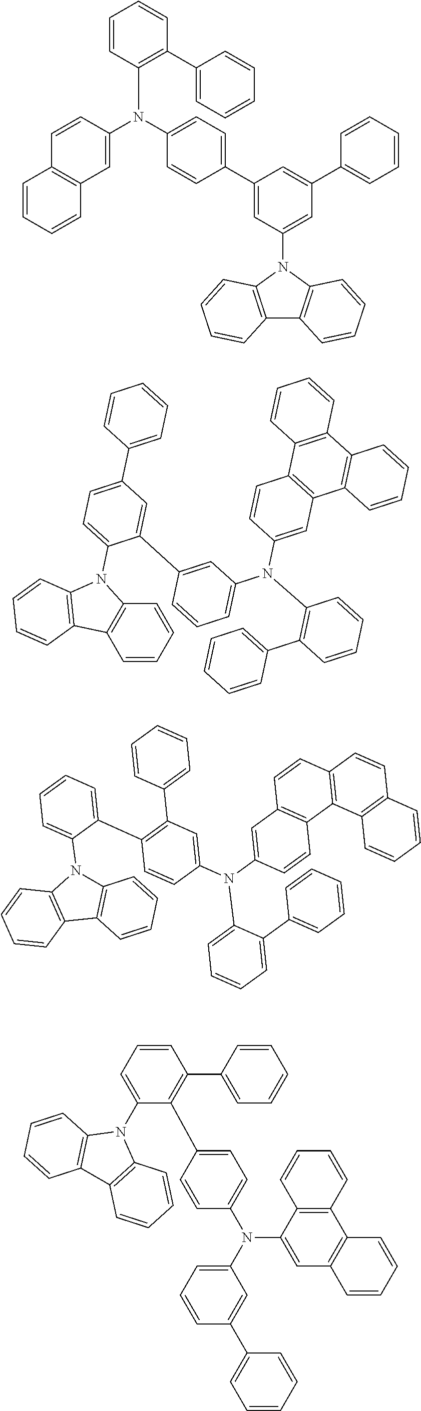

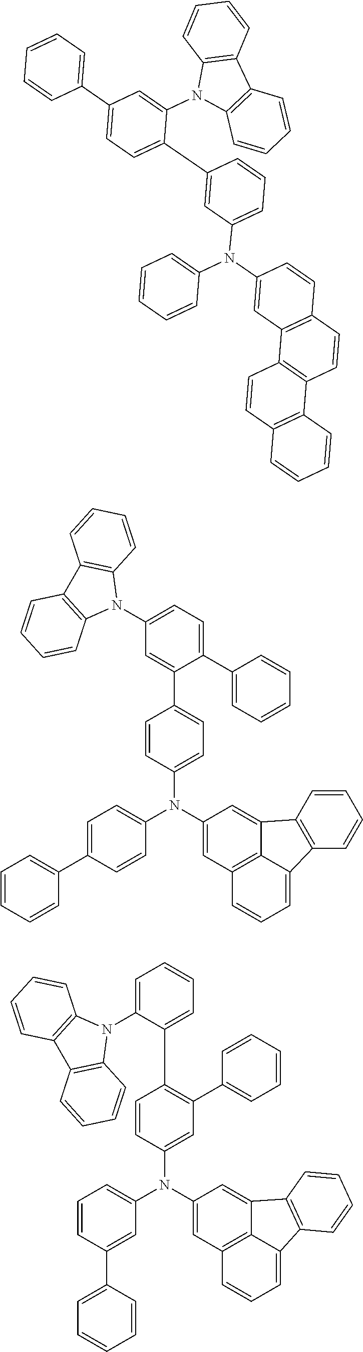 [SMILES: c1ccc(-c2ccc(-c3cccc(N(c4ccccc4)c4ccc5ccc6c7ccccc7ccc6c5c4)c3)c(-n3c4ccccc4c4ccccc43)c2)cc1 ; c1ccc(-c2ccc(N(c3ccc(-c4cc(-n5c6ccccc6c6ccccc65)ccc4-c4ccccc4)cc3)c3cc4c5c(cccc5c3)-c3ccccc3-4)cc2)cc1 ; c1ccc(-c2cccc(N(c3ccc(-c4ccccc4-n4c5ccccc5c5ccccc54)c(-c4ccccc4)c3)c3cc4c5c(cccc5c3)-c3ccccc3-4)c2)cc1]

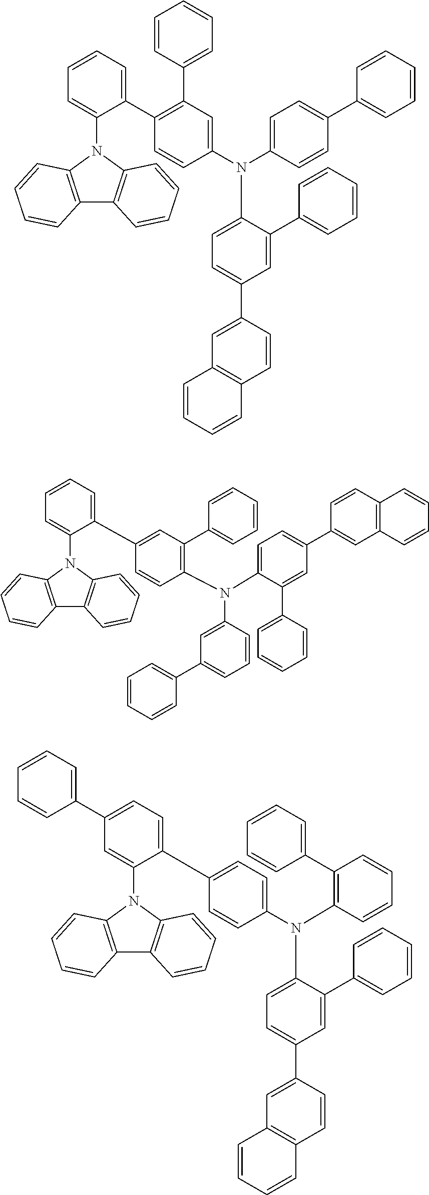 [SMILES: c1ccc(-c2ccc(N(c3ccc(-c4ccccc4-n4c5ccccc5c5ccccc54)c(-c4ccccc4)c3)c3ccc(-c4ccc5ccccc5c4)cc3-c3ccccc3)cc2)cc1 ; c1ccc(-c2cccc(N(c3ccc(-c4ccc5ccccc5c4)cc3-c3ccccc3)c3ccc(-c4ccccc4-n4c5ccccc5c5ccccc54)cc3-c3ccccc3)c2)cc1 ; c1ccc(-c2ccc(-c3ccc(N(c4ccccc4-c4ccccc4)c4ccc(-c5ccc6ccccc6c5)cc4-c4ccccc4)cc3)c(-n3c4ccccc4c4ccccc43)c2)cc1]

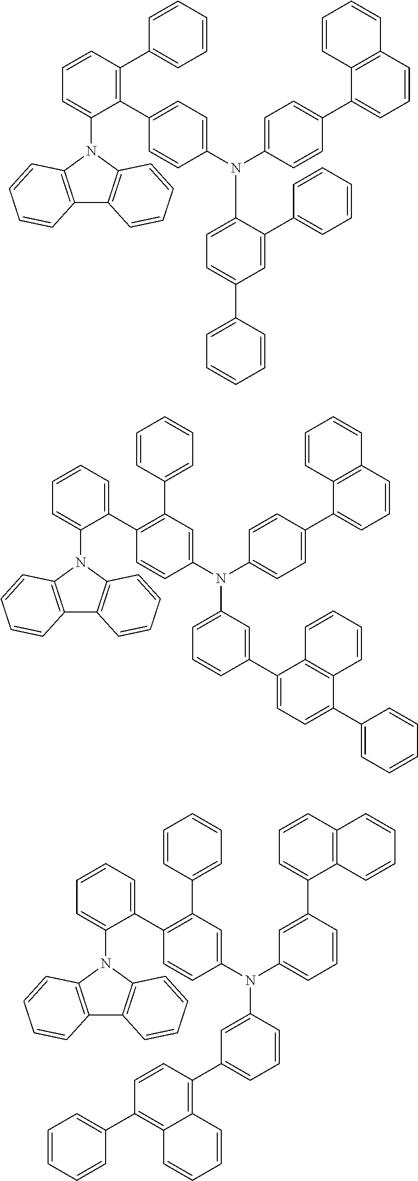 [SMILES: c1ccc(-c2ccc(N(c3ccc(-c4c(-c5ccccc5)cccc4-n4c5ccccc5c5ccccc54)cc3)c3ccc(-c4cccc5ccccc45)cc3)c(-c3ccccc3)c2)cc1 ; c1ccc(-c2cc(N(c3ccc(-c4cccc5ccccc45)cc3)c3cccc(-c4ccc(-c5ccccc5)c5ccccc45)c3)ccc2-c2ccccc2-n2c3ccccc3c3ccccc32)cc1 ; c1ccc(-c2cc(N(c3cccc(-c4cccc5ccccc45)c3)c3cccc(-c4ccc(-c5ccccc5)c5ccccc45)c3)ccc2-c2ccccc2-n2c3ccccc3c3ccccc32)cc1]

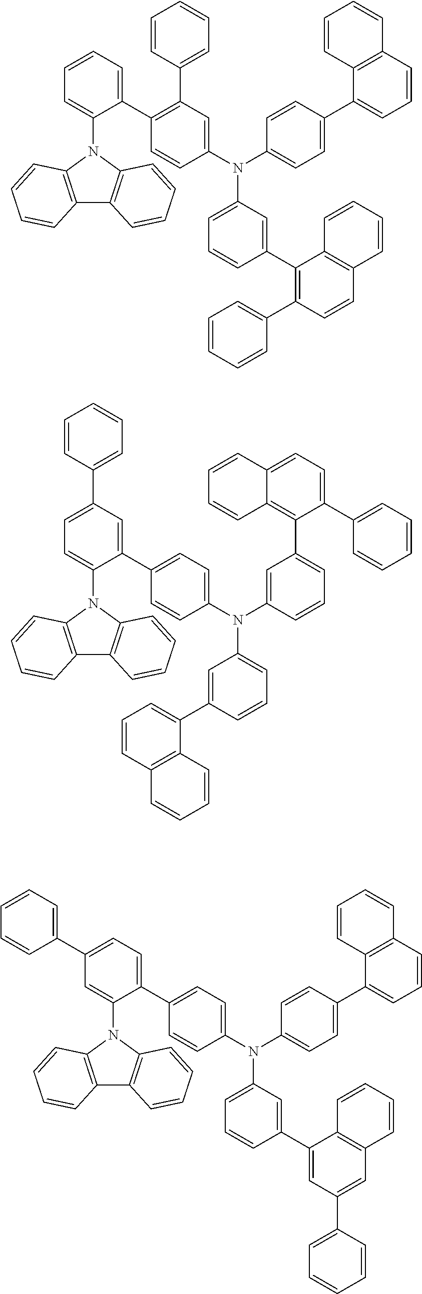 [SMILES: c1ccc(-c2cc(N(c3ccc(-c4cccc5ccccc45)cc3)c3cccc(-c4c(-c5ccccc5)ccc5ccccc45)c3)ccc2-c2ccccc2-n2c3ccccc3c3ccccc32)cc1 ; c1ccc(-c2ccc(-n3c4ccccc4c4ccccc43)c(-c3ccc(N(c4cccc(-c5cccc6ccccc56)c4)c4cccc(-c5c(-c6ccccc6)ccc6ccccc56)c4)cc3)c2)cc1 ; c1ccc(-c2ccc(-c3ccc(N(c4ccc(-c5cccc6ccccc56)cc4)c4cccc(-c5cc(-c6ccccc6)cc6ccccc56)c4)cc3)c(-n3c4ccccc4c4ccccc43)c2)cc1]

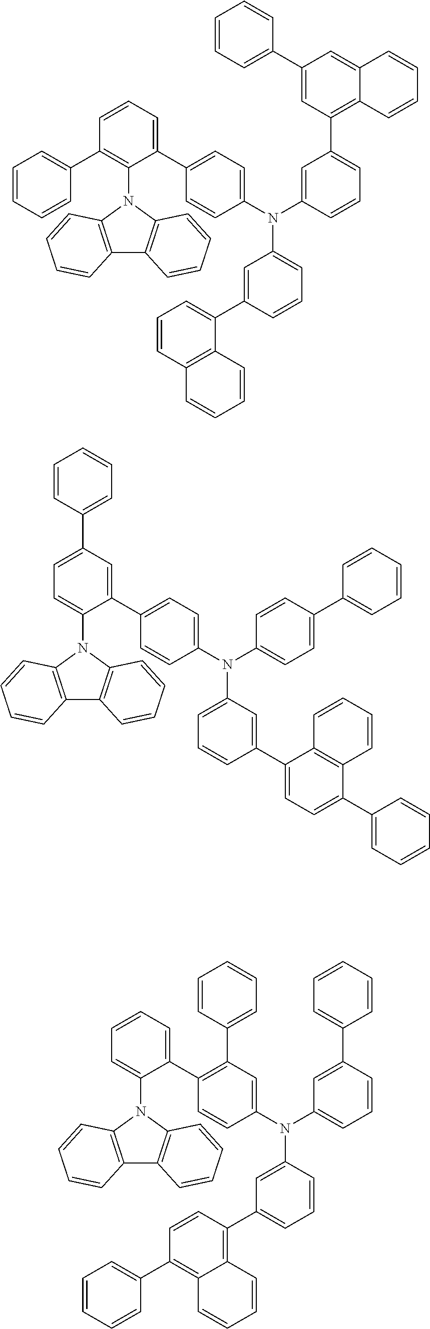 [SMILES: c1ccc(-c2cc(-c3cccc(N(c4ccc(-c5cccc(-c6ccccc6)c5-n5c6ccccc6c6ccccc65)cc4)c4cccc(-c5cccc6ccccc56)c4)c3)c3ccccc3c2)cc1 ; c1ccc(-c2ccc(N(c3ccc(-c4cc(-c5ccccc5)ccc4-n4c5ccccc5c5ccccc54)cc3)c3cccc(-c4ccc(-c5ccccc5)c5ccccc45)c3)cc2)cc1 ; c1ccc(-c2cccc(N(c3cccc(-c4ccc(-c5ccccc5)c5ccccc45)c3)c3ccc(-c4ccccc4-n4c5ccccc5c5ccccc54)c(-c4ccccc4)c3)c2)cc1]

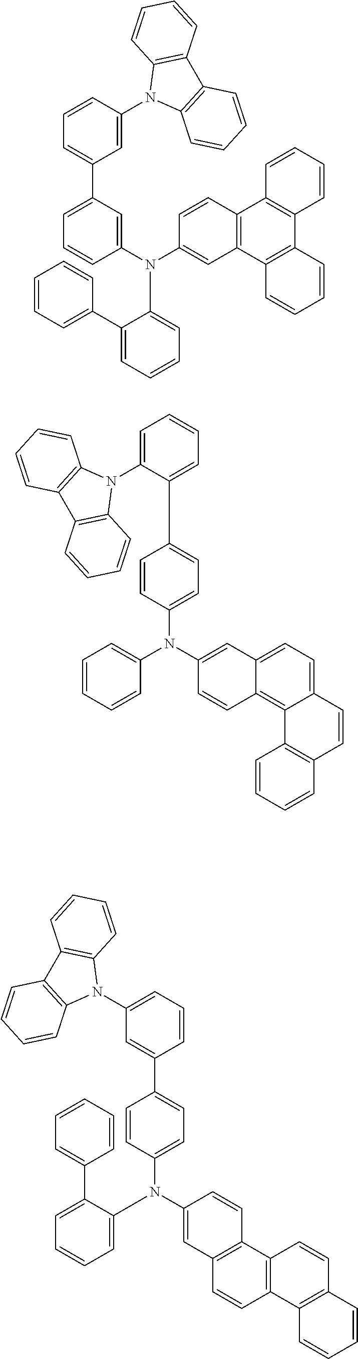

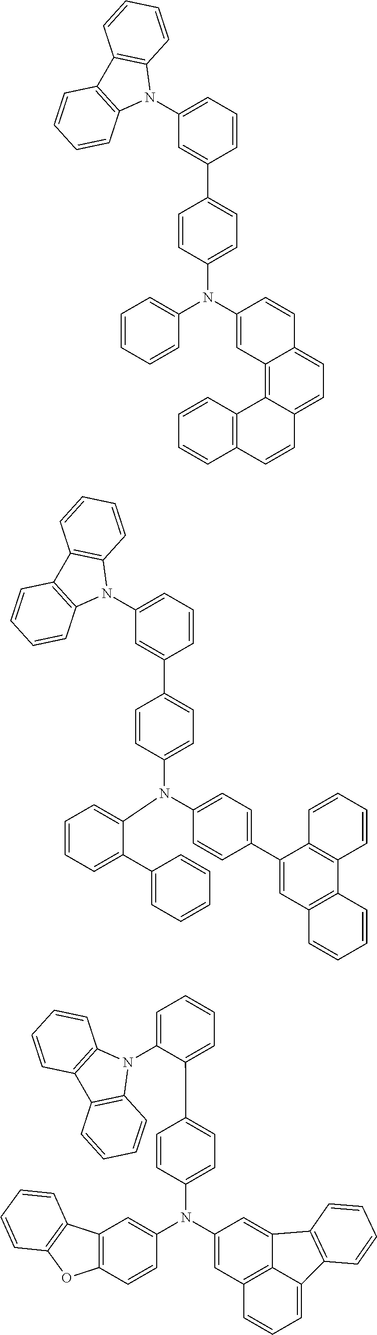 [SMILES: c1ccc(N(c2ccc(-c3cccc(-n4c5ccccc5c5ccccc54)c3)cc2)c2ccc3ccc4ccc5ccccc5c4c3c2)cc1 ; c1ccc(-c2ccccc2N(c2ccc(-c3cccc(-n4c5ccccc5c5ccccc54)c3)cc2)c2ccc(-c3cc4ccccc4c4ccccc34)cc2)cc1 ; c1ccc2c(c1)-c1cccc3cc(N(c4ccc(-c5ccccc5-n5c6ccccc6c6ccccc65)cc4)c4ccc5oc6ccccc6c5c4)cc-2c13]

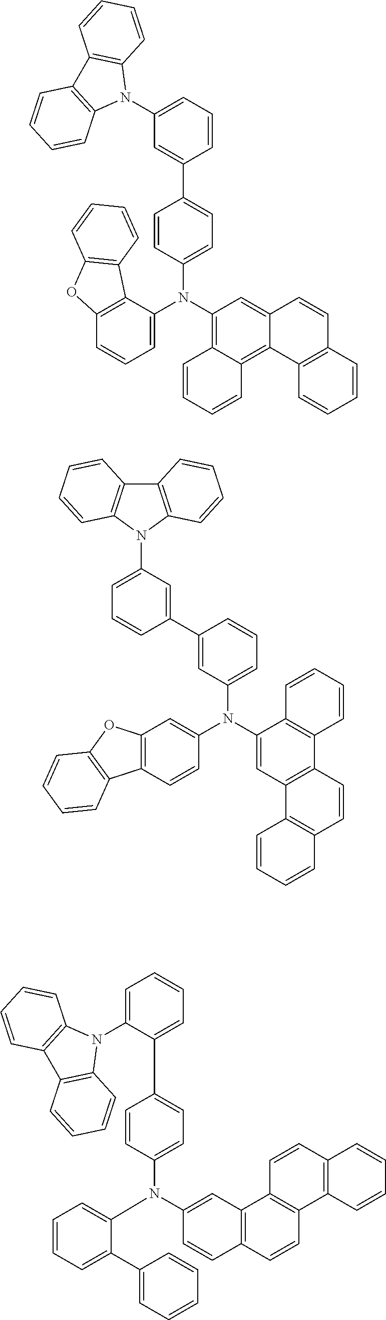 [SMILES: c1cc(-c2ccc(N(c3cc4ccc5ccccc5c4c4ccccc34)c3cccc4oc5ccccc5c34)cc2)cc(-n2c3ccccc3c3ccccc32)c1 ; c1cc(-c2cccc(-n3c4ccccc4c4ccccc43)c2)cc(N(c2ccc3c(c2)oc2ccccc23)c2cc3c4ccccc4ccc3c3ccccc23)c1 ; c1ccc(-c2ccccc2N(c2ccc(-c3ccccc3-n3c4ccccc4c4ccccc43)cc2)c2ccc3ccc4c5ccccc5ccc4c3c2)cc1]

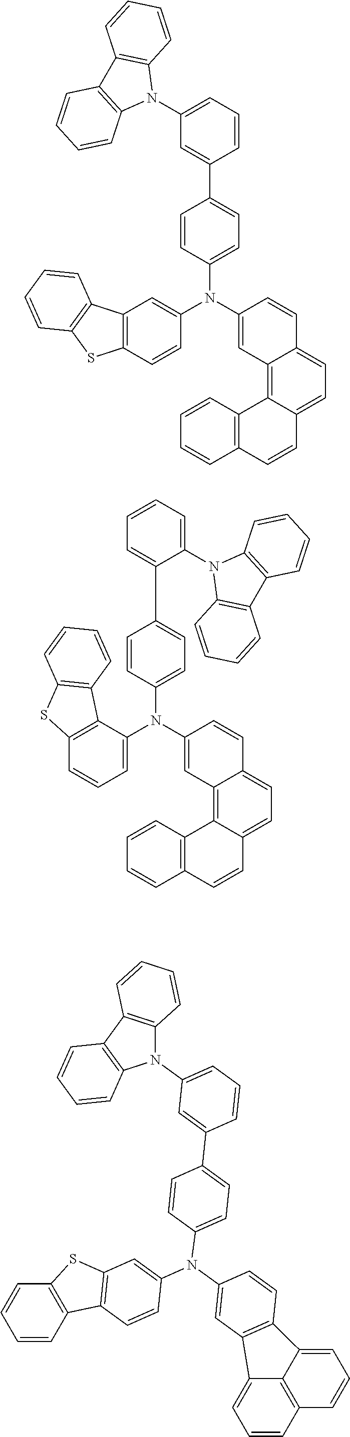 [SMILES: c1cc(-c2ccc(N(c3ccc4sc5ccccc5c4c3)c3ccc4ccc5ccc6ccccc6c5c4c3)cc2)cc(-n2c3ccccc3c3ccccc32)c1 ; c1ccc(-n2c3ccccc3c3ccccc32)c(-c2ccc(N(c3ccc4ccc5ccc6ccccc6c5c4c3)c3cccc4sc5ccccc5c34)cc2)c1 ; c1cc(-c2ccc(N(c3ccc4c(c3)-c3cccc5cccc-4c35)c3ccc4c(c3)sc3ccccc34)cc2)cc(-n2c3ccccc3c3ccccc32)c1]

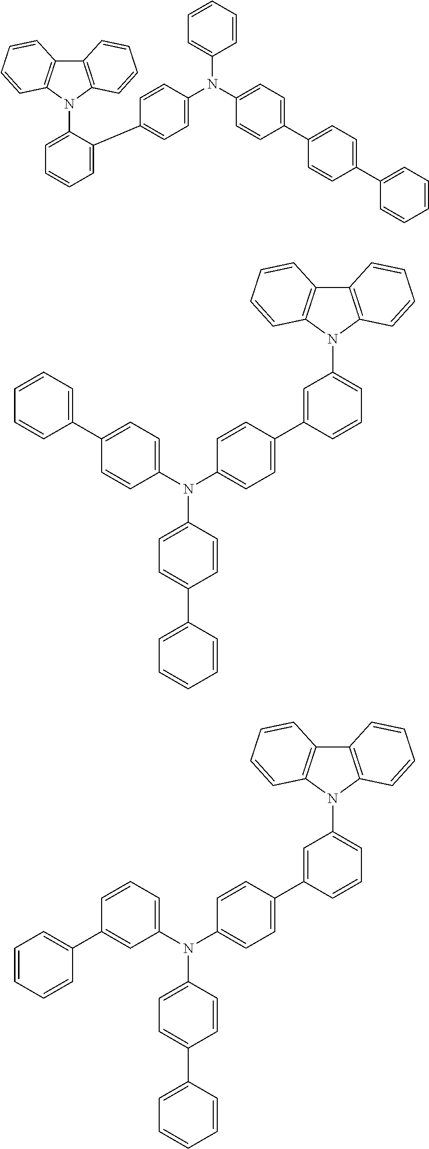 [SMILES: c1ccc(-c2ccc(-c3ccc(N(c4ccccc4)c4ccc(-c5ccccc5-n5c6ccccc6c6ccccc65)cc4)cc3)cc2)cc1 ; c1ccc(-c2ccc(N(c3ccc(-c4ccccc4)cc3)c3ccc(-c4cccc(-n5c6ccccc6c6ccccc65)c4)cc3)cc2)cc1 ; c1ccc(-c2ccc(N(c3ccc(-c4cccc(-n5c6ccccc6c6ccccc65)c4)cc3)c3cccc(-c4ccccc4)c3)cc2)cc1]

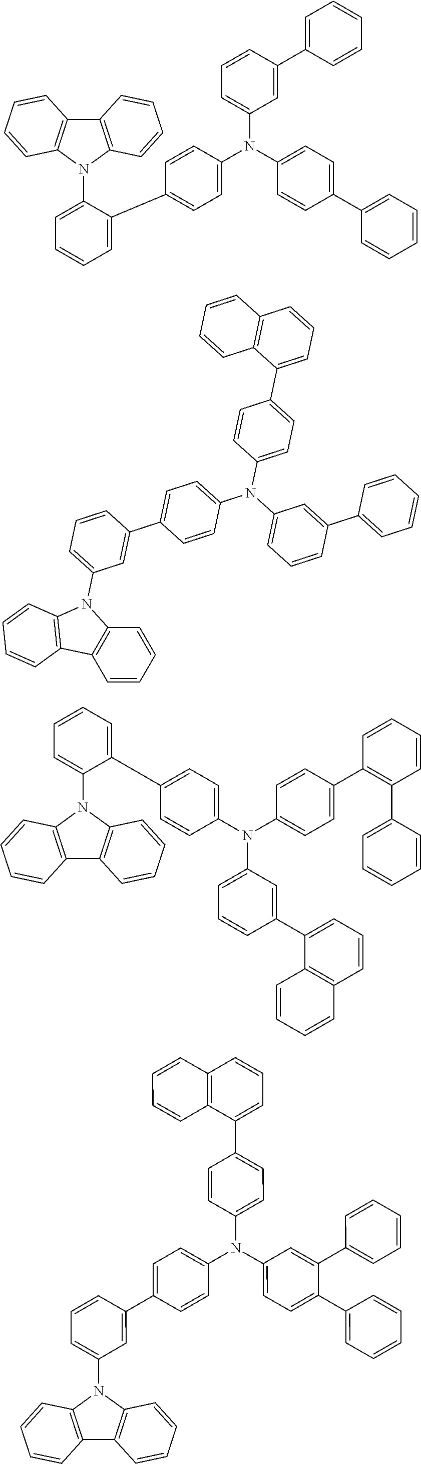

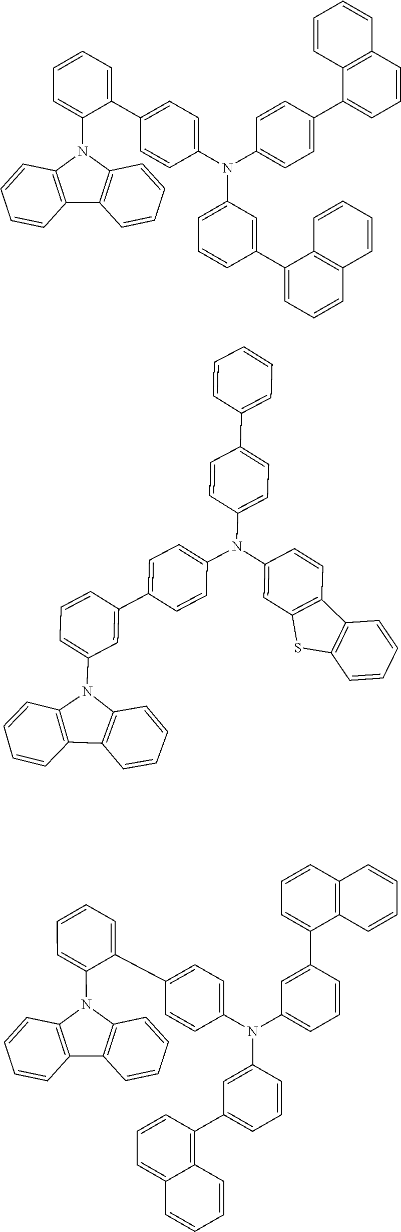 [SMILES: c1cc(-c2cccc3ccccc23)cc(N(c2ccc(-c3ccccc3-n3c4ccccc4c4ccccc43)cc2)c2ccc(-c3cccc4ccccc34)cc2)c1 ; c1ccc(-c2ccc(N(c3ccc(-c4cccc(-n5c6ccccc6c6ccccc65)c4)cc3)c3ccc4c(c3)sc3ccccc34)cc2)cc1 ; c1cc(-c2cccc3ccccc23)cc(N(c2ccc(-c3ccccc3-n3c4ccccc4c4ccccc43)cc2)c2cccc(-c3cccc4ccccc34)c2)c1]

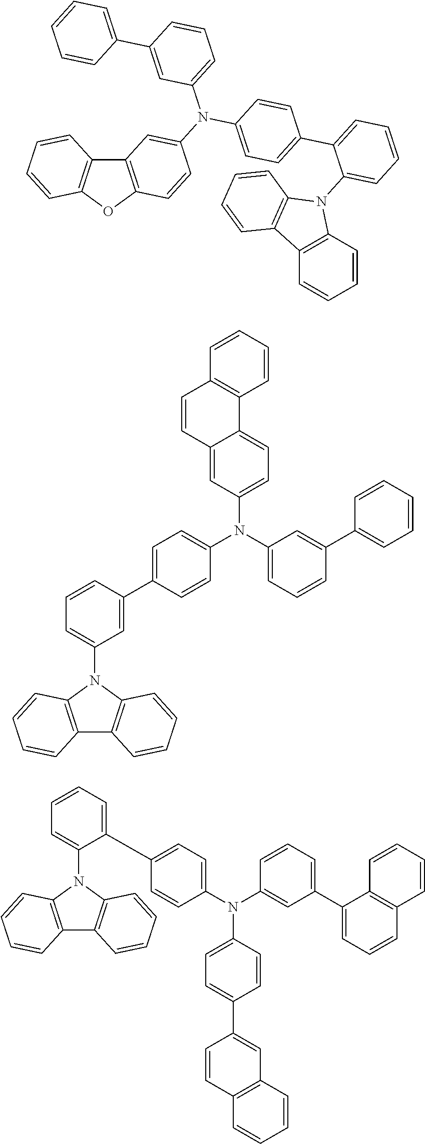

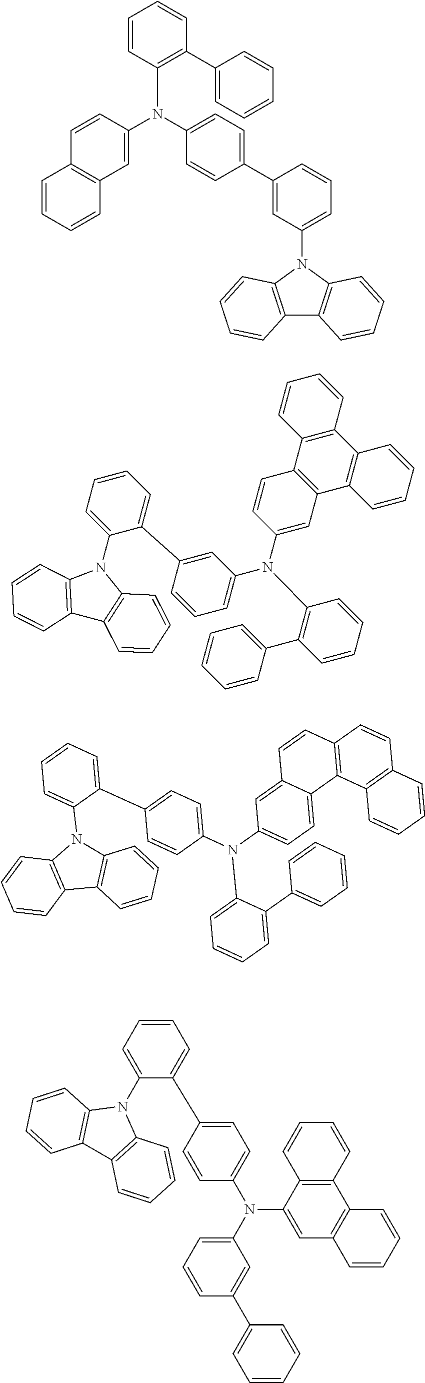

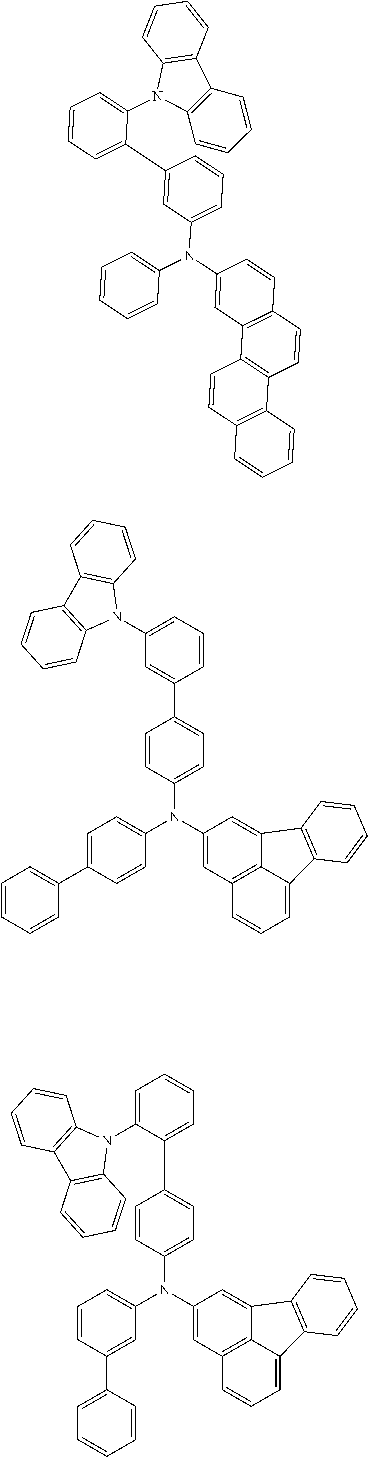 [SMILES: c1ccc(N(c2cccc(-c3ccccc3-n3c4ccccc4c4ccccc43)c2)c2ccc3ccc4c5ccccc5ccc4c3c2)cc1 ; c1ccc(-c2ccc(N(c3ccc(-c4cccc(-n5c6ccccc6c6ccccc65)c4)cc3)c3cc4c5c(cccc5c3)-c3ccccc3-4)cc2)cc1 ; c1ccc(-c2cccc(N(c3ccc(-c4ccccc4-n4c5ccccc5c5ccccc54)cc3)c3cc4c5c(cccc5c3)-c3ccccc3-4)c2)cc1]

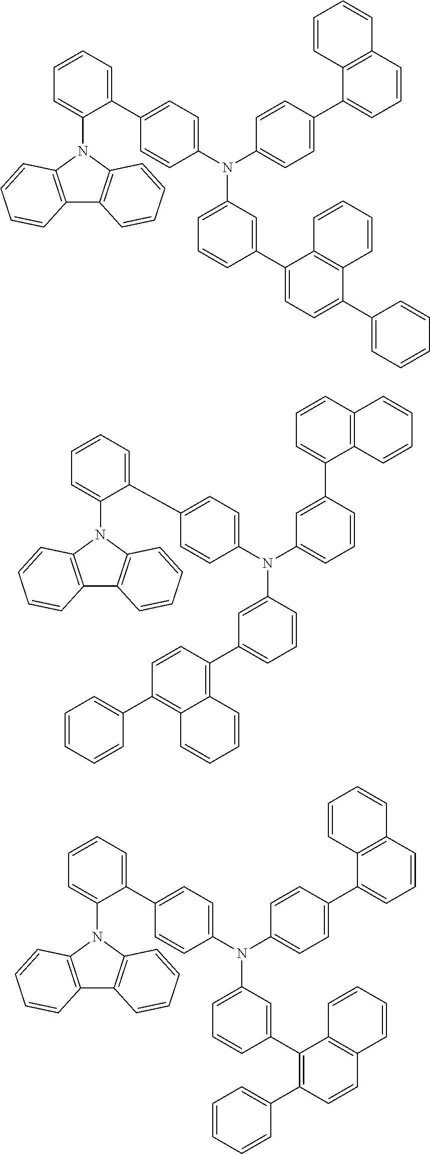

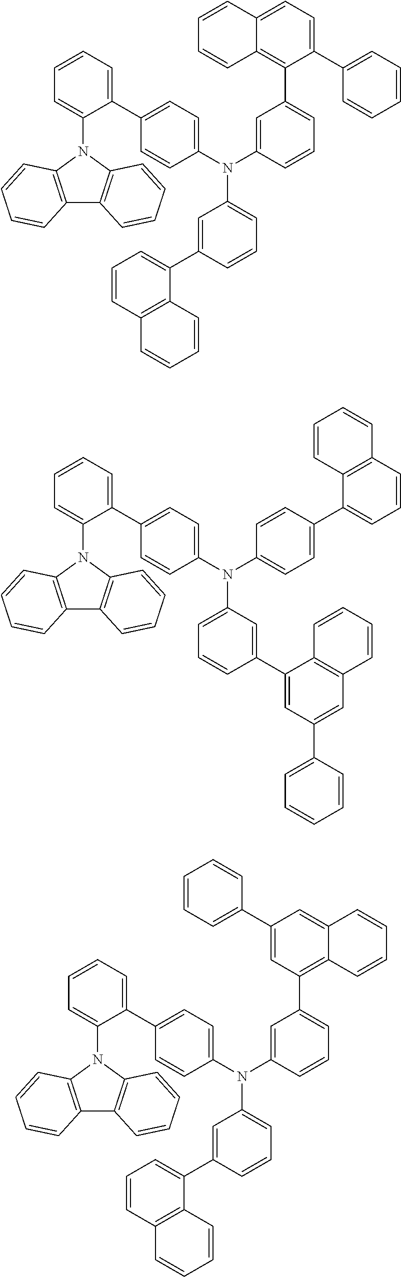 [SMILES: c1ccc(-c2ccc3ccccc3c2-c2cccc(N(c3ccc(-c4ccccc4-n4c5ccccc5c5ccccc54)cc3)c3cccc(-c4cccc5ccccc45)c3)c2)cc1 ; c1ccc(-c2cc(-c3cccc(N(c4ccc(-c5ccccc5-n5c6ccccc6c6ccccc65)cc4)c4ccc(-c5cccc6ccccc56)cc4)c3)c3ccccc3c2)cc1 ; c1ccc(-c2cc(-c3cccc(N(c4ccc(-c5ccccc5-n5c6ccccc6c6ccccc65)cc4)c4cccc(-c5cccc6ccccc56)c4)c3)c3ccccc3c2)cc1]

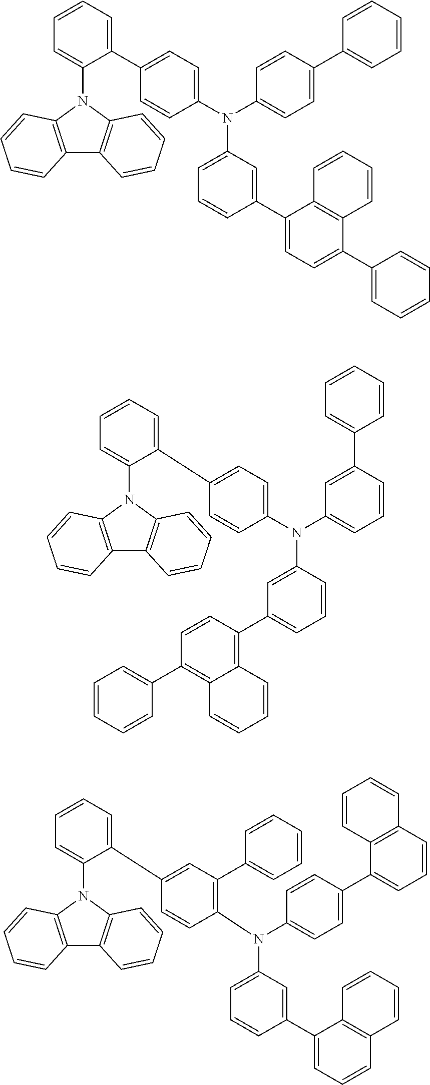 [SMILES: c1ccc(-c2ccc(N(c3ccc(-c4ccccc4-n4c5ccccc5c5ccccc54)cc3)c3cccc(-c4ccc(-c5ccccc5)c5ccccc45)c3)cc2)cc1 ; c1ccc(-c2cccc(N(c3ccc(-c4ccccc4-n4c5ccccc5c5ccccc54)cc3)c3cccc(-c4ccc(-c5ccccc5)c5ccccc45)c3)c2)cc1 ; c1ccc(-c2cc(-c3ccccc3-n3c4ccccc4c4ccccc43)ccc2N(c2ccc(-c3cccc4ccccc34)cc2)c2cccc(-c3cccc4ccccc34)c2)cc1]

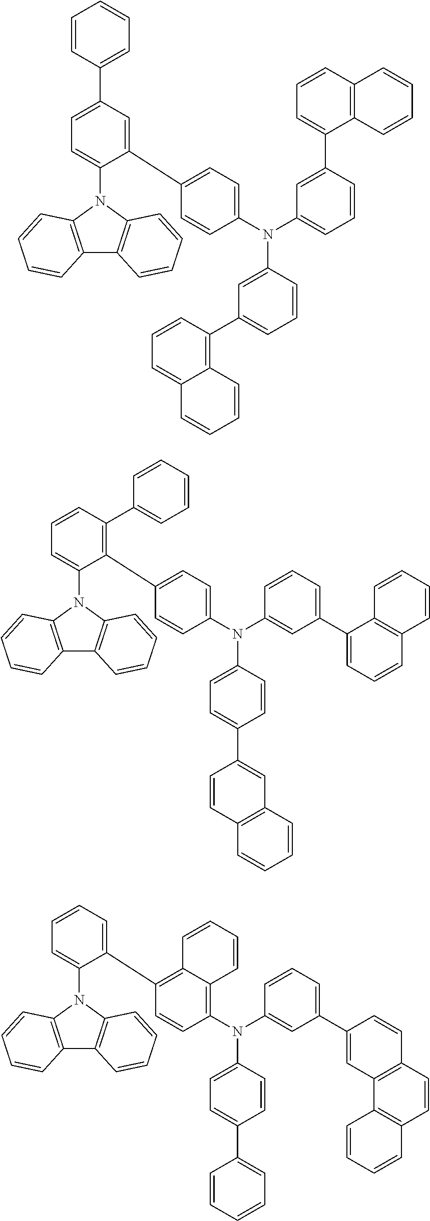 [SMILES: c1ccc(-c2ccc(-n3c4ccccc4c4ccccc43)c(-c3ccc(N(c4cccc(-c5cccc6ccccc56)c4)c4cccc(-c5cccc6ccccc56)c4)cc3)c2)cc1 ; c1ccc(-c2cccc(-n3c4ccccc4c4ccccc43)c2-c2ccc(N(c3ccc(-c4ccc5ccccc5c4)cc3)c3cccc(-c4cccc5ccccc45)c3)cc2)cc1 ; c1ccc(-c2ccc(N(c3cccc(-c4ccc5ccc6ccccc6c5c4)c3)c3ccc(-c4ccccc4-n4c5ccccc5c5ccccc54)c4ccccc34)cc2)cc1]

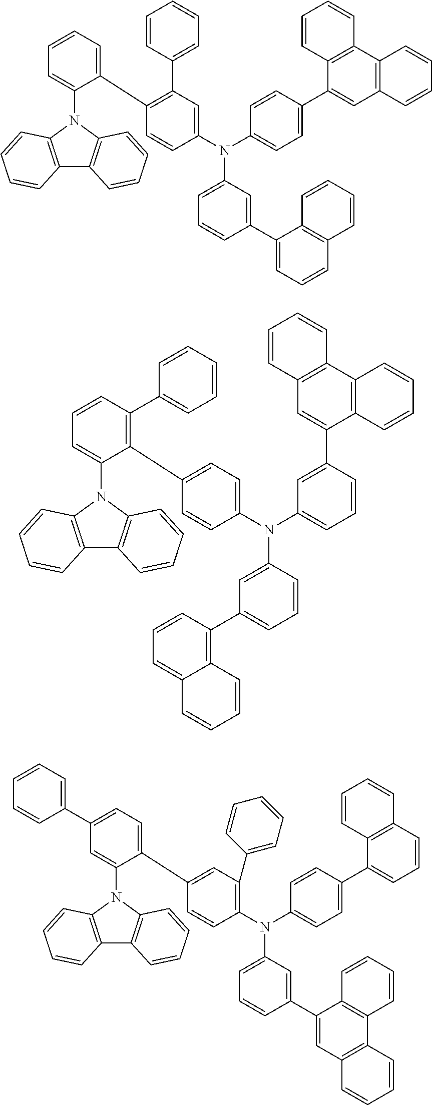

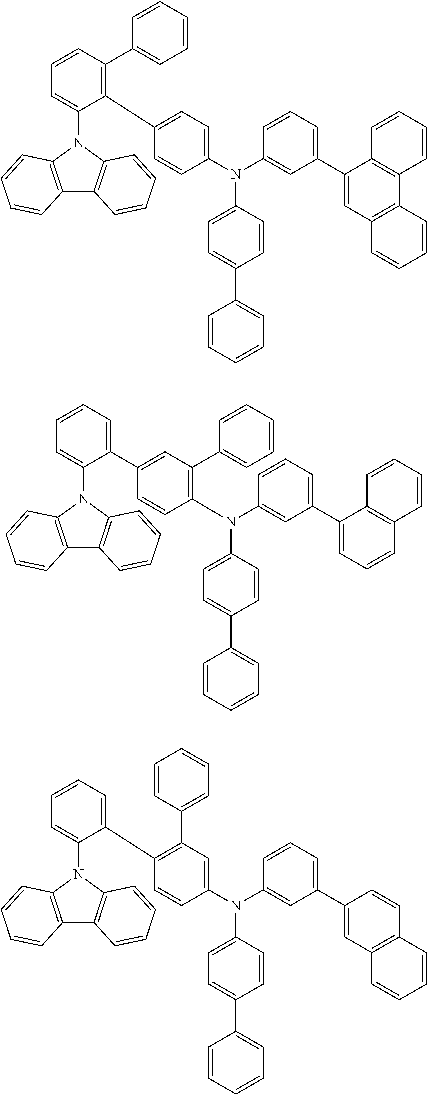

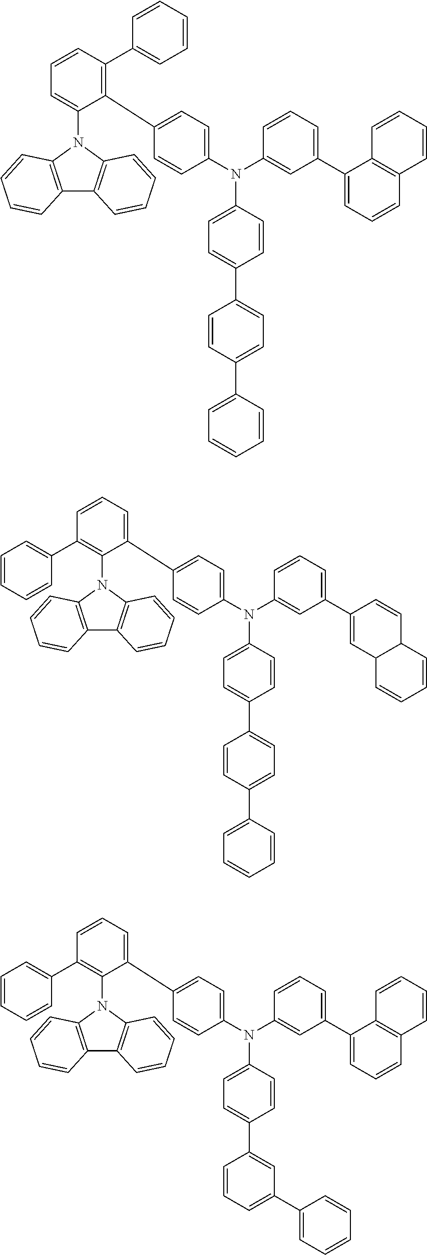 [SMILES: c1ccc(-c2ccc(-c3ccc(N(c4ccc(-c5c(-c6ccccc6)cccc5-n5c6ccccc6c6ccccc65)cc4)c4cccc(-c5cccc6ccccc56)c4)cc3)cc2)cc1 ; C1=CC2C=CC(c3cccc(N(c4ccc(-c5ccc(-c6ccccc6)cc5)cc4)c4ccc(-c5cccc(-c6ccccc6)c5-n5c6ccccc6c6ccccc65)cc4)c3)=CC2C=C1 ; c1ccc(-c2cccc(-c3ccc(N(c4ccc(-c5cccc(-c6ccccc6)c5-n5c6ccccc6c6ccccc65)cc4)c4cccc(-c5cccc6ccccc56)c4)cc3)c2)cc1]

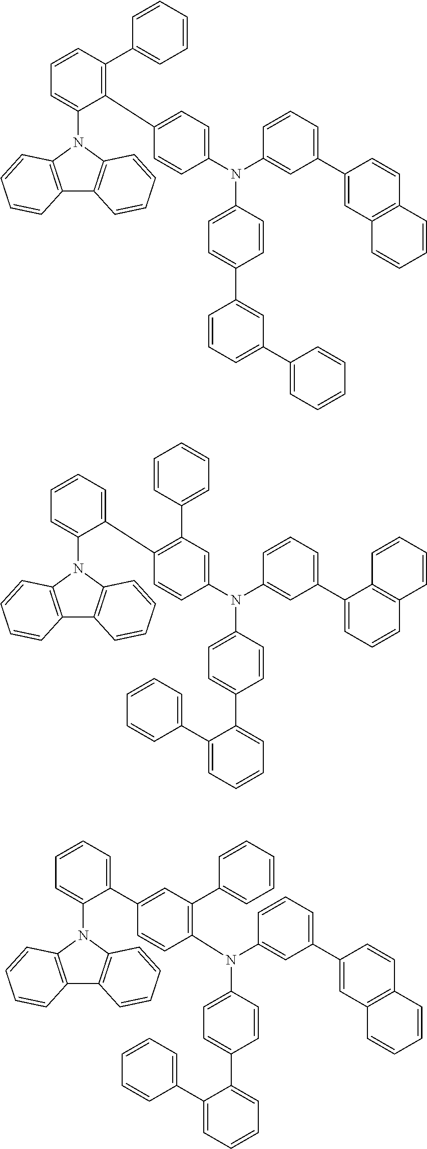 [SMILES: c1ccc(-c2cccc(-c3ccc(N(c4ccc(-c5c(-c6ccccc6)cccc5-n5c6ccccc6c6ccccc65)cc4)c4cccc(-c5ccc6ccccc6c5)c4)cc3)c2)cc1 ; c1ccc(-c2ccccc2-c2ccc(N(c3cccc(-c4cccc5ccccc45)c3)c3ccc(-c4ccccc4-n4c5ccccc5c5ccccc54)c(-c4ccccc4)c3)cc2)cc1 ; c1ccc(-c2ccccc2-c2ccc(N(c3cccc(-c4ccc5ccccc5c4)c3)c3ccc(-c4ccccc4-n4c5ccccc5c5ccccc54)cc3-c3ccccc3)cc2)cc1]

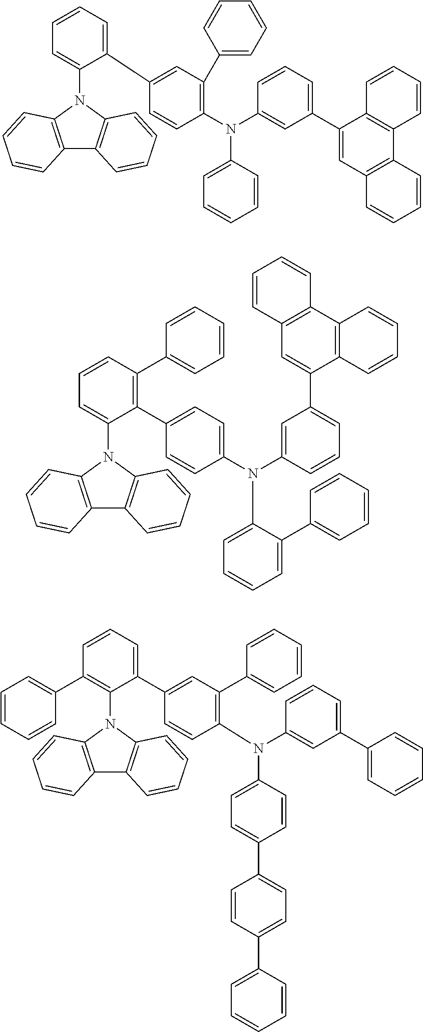 [SMILES: c1ccc(-c2cc(-c3ccccc3-n3c4ccccc4c4ccccc43)ccc2N(c2ccccc2)c2cccc(-c3cc4ccccc4c4ccccc34)c2)cc1 ; c1ccc(-c2ccccc2N(c2ccc(-c3c(-c4ccccc4)cccc3-n3c4ccccc4c4ccccc43)cc2)c2cccc(-c3cc4ccccc4c4ccccc34)c2)cc1 ; c1ccc(-c2ccc(-c3ccc(N(c4cccc(-c5ccccc5)c4)c4ccc(-c5cccc(-c6ccccc6)c5-n5c6ccccc6c6ccccc65)cc4-c4ccccc4)cc3)cc2)cc1]

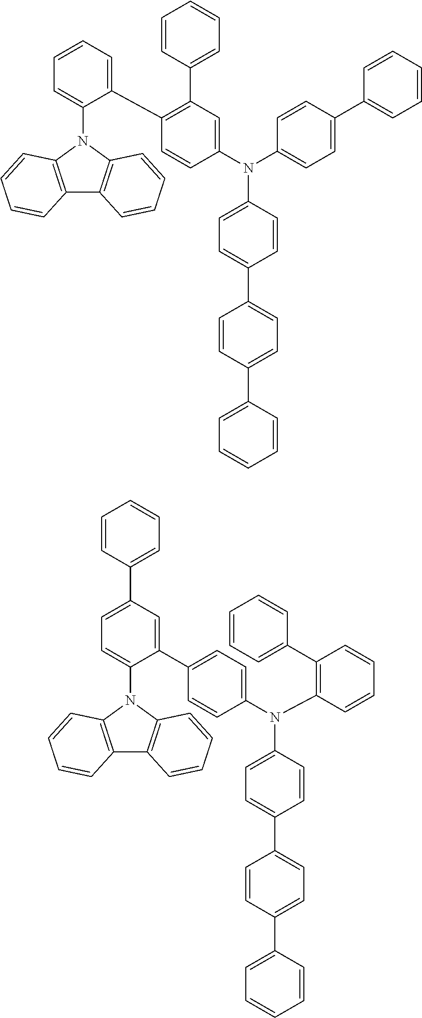 [SMILES: c1ccc(-c2ccc(-c3ccc(N(c4ccc(-c5ccccc5)cc4)c4ccc(-c5ccccc5-n5c6ccccc6c6ccccc65)c(-c5ccccc5)c4)cc3)cc2)cc1 ; c1ccc(-c2ccc(-c3ccc(N(c4ccc(-c5cc(-c6ccccc6)ccc5-n5c6ccccc6c6ccccc65)cc4)c4ccccc4-c4ccccc4)cc3)cc2)cc1]

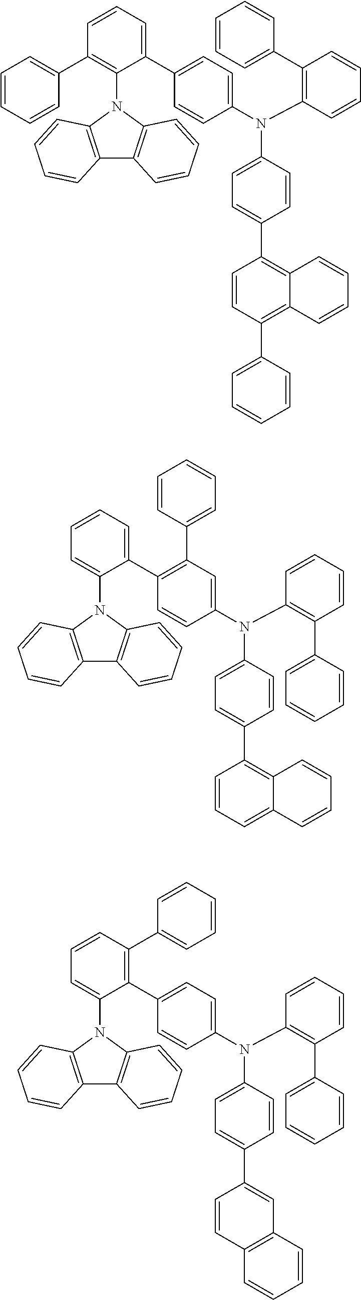 [SMILES: c1ccc(-c2ccccc2N(c2ccc(-c3cccc(-c4ccccc4)c3-n3c4ccccc4c4ccccc43)cc2)c2ccc(-c3ccc(-c4ccccc4)c4ccccc34)cc2)cc1 ; c1ccc(-c2cc(N(c3ccc(-c4cccc5ccccc45)cc3)c3ccccc3-c3ccccc3)ccc2-c2ccccc2-n2c3ccccc3c3ccccc32)cc1 ; c1ccc(-c2ccccc2N(c2ccc(-c3ccc4ccccc4c3)cc2)c2ccc(-c3c(-c4ccccc4)cccc3-n3c4ccccc4c4ccccc43)cc2)cc1]

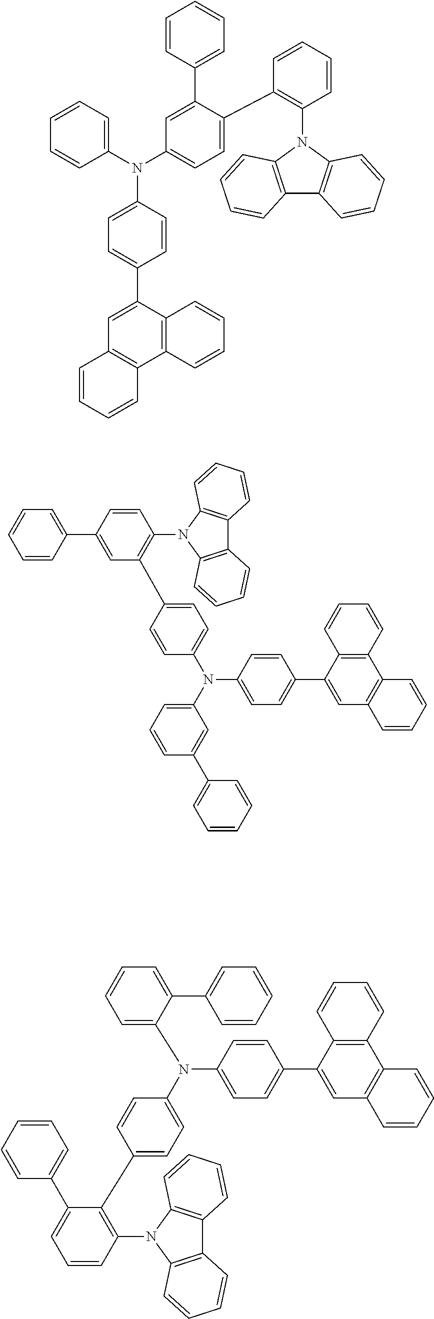 [SMILES: c1ccc(-c2cc(N(c3ccccc3)c3ccc(-c4cc5ccccc5c5ccccc45)cc3)ccc2-c2ccccc2-n2c3ccccc3c3ccccc32)cc1 ; c1ccc(-c2cccc(N(c3ccc(-c4cc(-c5ccccc5)ccc4-n4c5ccccc5c5ccccc54)cc3)c3ccc(-c4cc5ccccc5c5ccccc45)cc3)c2)cc1 ; c1ccc(-c2ccccc2N(c2ccc(-c3c(-c4ccccc4)cccc3-n3c4ccccc4c4ccccc43)cc2)c2ccc(-c3cc4ccccc4c4ccccc34)cc2)cc1]

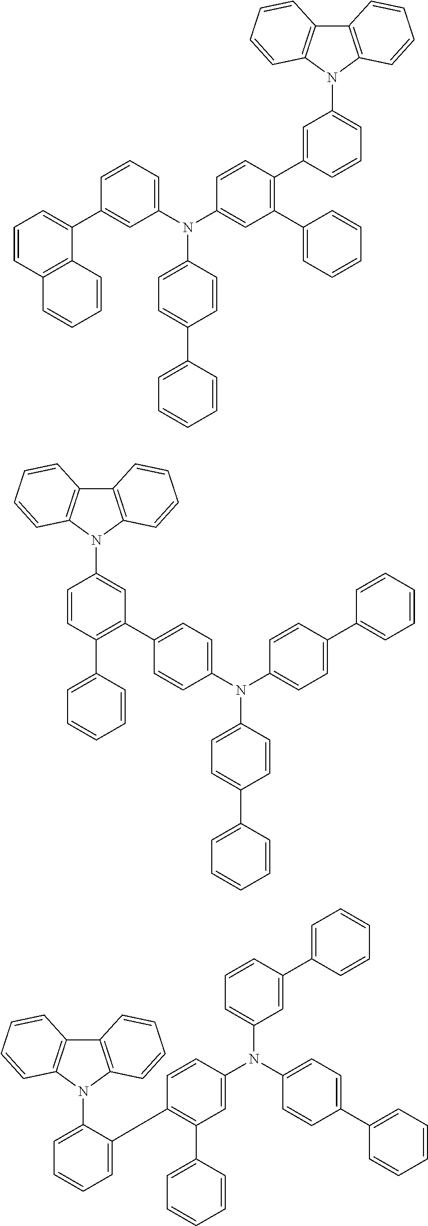 [SMILES: c1ccc(-c2ccc(N(c3cccc(-c4cccc5ccccc45)c3)c3ccc(-c4cccc(-n5c6ccccc6c6ccccc65)c4)c(-c4ccccc4)c3)cc2)cc1 ; c1ccc(-c2ccc(N(c3ccc(-c4ccccc4)cc3)c3ccc(-c4cc(-n5c6ccccc6c6ccccc65)ccc4-c4ccccc4)cc3)cc2)cc1 ; c1ccc(-c2ccc(N(c3cccc(-c4ccccc4)c3)c3ccc(-c4ccccc4-n4c5ccccc5c5ccccc54)c(-c4ccccc4)c3)cc2)cc1]

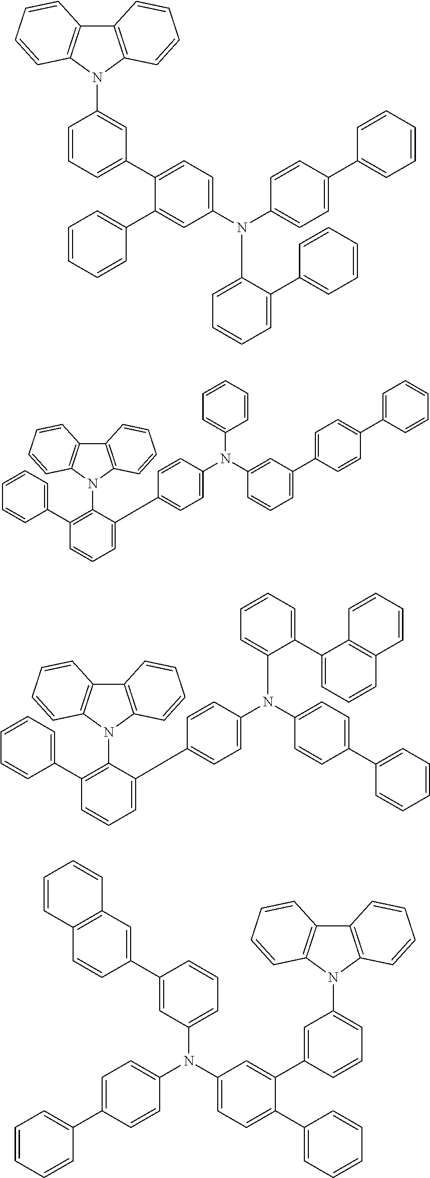

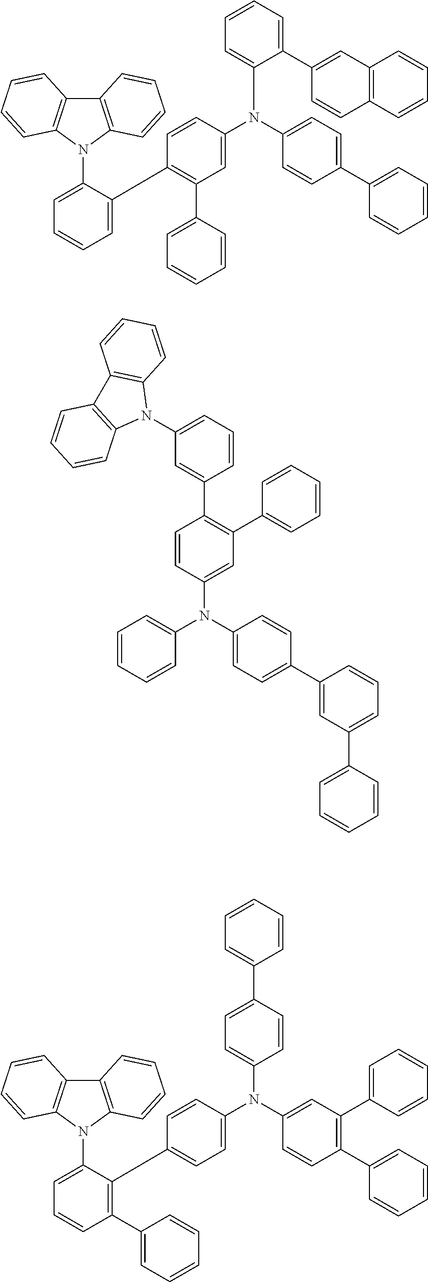 [SMILES: c1ccc(-c2ccc(N(c3ccc(-c4ccccc4-n4c5ccccc5c5ccccc54)c(-c4ccccc4)c3)c3ccccc3-c3ccc4ccccc4c3)cc2)cc1 ; c1ccc(-c2cccc(-c3ccc(N(c4ccccc4)c4ccc(-c5cccc(-n6c7ccccc7c7ccccc76)c5)c(-c5ccccc5)c4)cc3)c2)cc1 ; c1ccc(-c2ccc(N(c3ccc(-c4c(-c5ccccc5)cccc4-n4c5ccccc5c5ccccc54)cc3)c3ccc(-c4ccccc4)c(-c4ccccc4)c3)cc2)cc1]

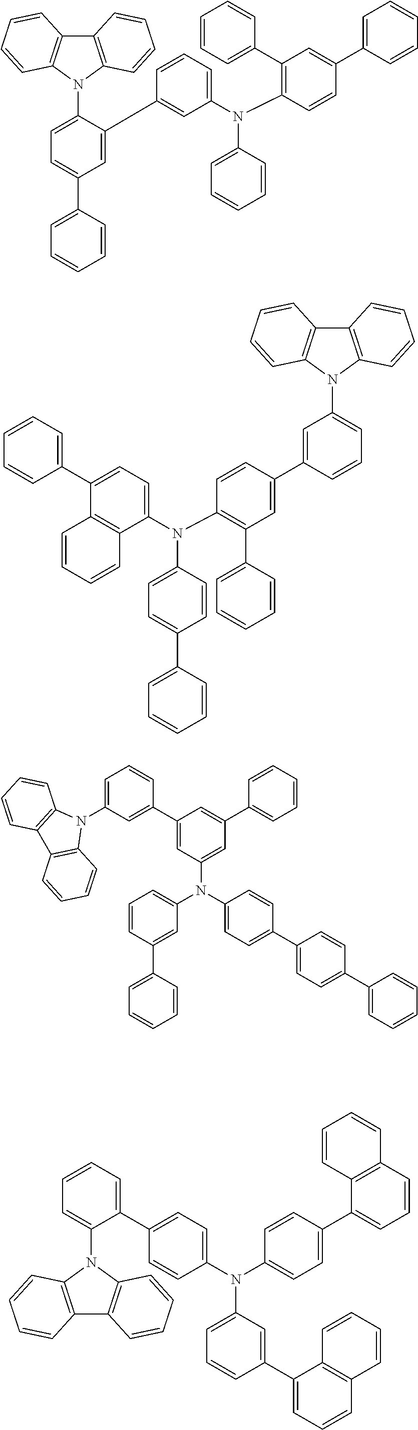 [SMILES: c1ccc(-c2ccc(N(c3ccccc3)c3cccc(-c4cc(-c5ccccc5)ccc4-n4c5ccccc5c5ccccc54)c3)c(-c3ccccc3)c2)cc1 ; c1ccc(-c2ccc(N(c3ccc(-c4cccc(-n5c6ccccc6c6ccccc65)c4)cc3-c3ccccc3)c3ccc(-c4ccccc4)c4ccccc34)cc2)cc1 ; c1ccc(-c2ccc(-c3ccc(N(c4cccc(-c5ccccc5)c4)c4cc(-c5ccccc5)cc(-c5cccc(-n6c7ccccc7c7ccccc76)c5)c4)cc3)cc2)cc1 ; c1cc(-c2cccc3ccccc23)cc(N(c2ccc(-c3ccccc3-n3c4ccccc4c4ccccc43)cc2)c2ccc(-c3cccc4ccccc34)cc2)c1]

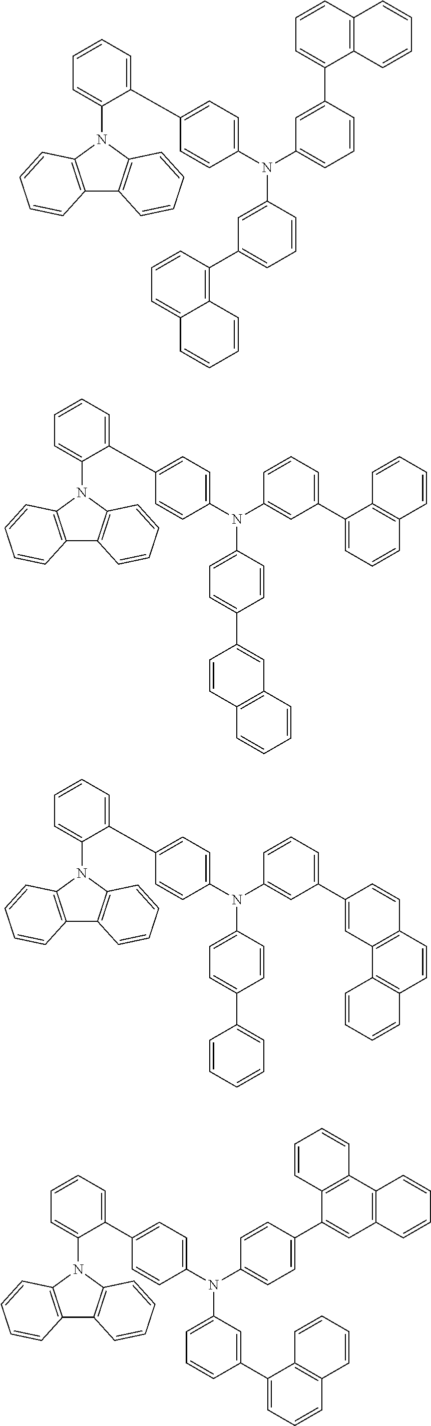 [SMILES: c1cc(-c2cccc3ccccc23)cc(N(c2ccc(-c3ccccc3-n3c4ccccc4c4ccccc43)cc2)c2cccc(-c3cccc4ccccc34)c2)c1 ; c1cc(-c2cccc3ccccc23)cc(N(c2ccc(-c3ccc4ccccc4c3)cc2)c2ccc(-c3ccccc3-n3c4ccccc4c4ccccc43)cc2)c1 ; c1ccc(-c2ccc(N(c3ccc(-c4ccccc4-n4c5ccccc5c5ccccc54)cc3)c3cccc(-c4ccc5ccc6ccccc6c5c4)c3)cc2)cc1 ; c1cc(-c2cccc3ccccc23)cc(N(c2ccc(-c3ccccc3-n3c4ccccc4c4ccccc43)cc2)c2ccc(-c3cc4ccccc4c4ccccc34)cc2)c1]

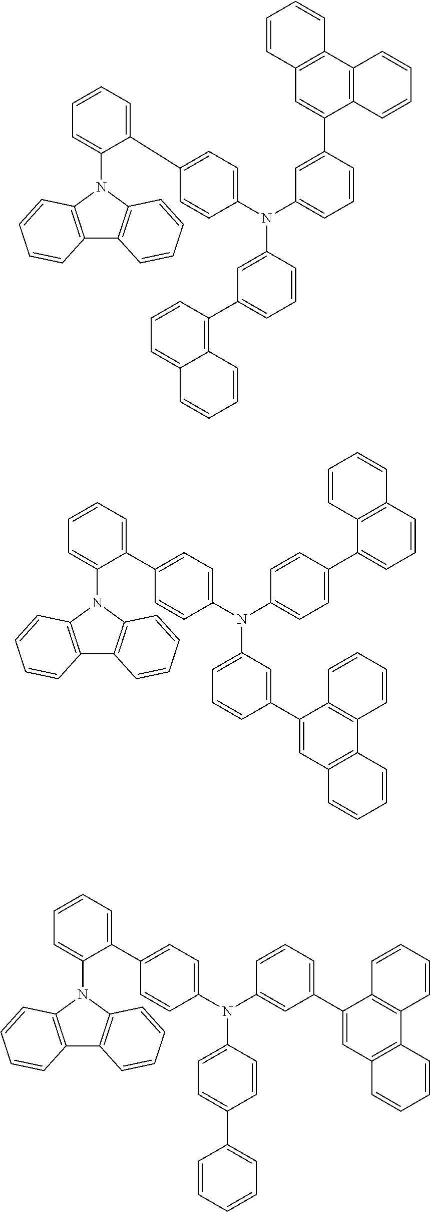 [SMILES: c1cc(-c2cccc3ccccc23)cc(N(c2ccc(-c3ccccc3-n3c4ccccc4c4ccccc43)cc2)c2cccc(-c3cc4ccccc4c4ccccc34)c2)c1 ; c1cc(-c2cc3ccccc3c3ccccc23)cc(N(c2ccc(-c3ccccc3-n3c4ccccc4c4ccccc43)cc2)c2ccc(-c3cccc4ccccc34)cc2)c1 ; c1ccc(-c2ccc(N(c3ccc(-c4ccccc4-n4c5ccccc5c5ccccc54)cc3)c3cccc(-c4cc5ccccc5c5ccccc45)c3)cc2)cc1]

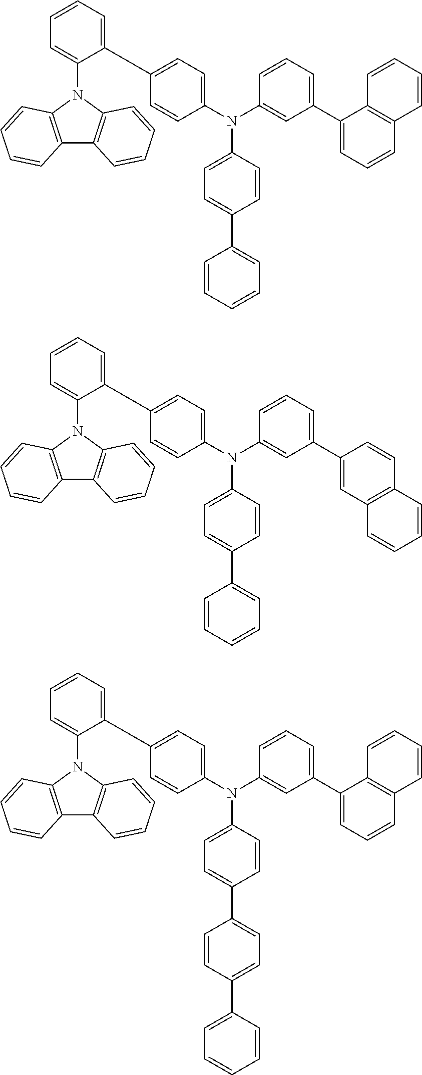 [SMILES: c1ccc(-c2ccc(N(c3ccc(-c4ccccc4-n4c5ccccc5c5ccccc54)cc3)c3cccc(-c4cccc5ccccc45)c3)cc2)cc1 ; c1ccc(-c2ccc(N(c3ccc(-c4ccccc4-n4c5ccccc5c5ccccc54)cc3)c3cccc(-c4ccc5ccccc5c4)c3)cc2)cc1 ; c1ccc(-c2ccc(-c3ccc(N(c4ccc(-c5ccccc5-n5c6ccccc6c6ccccc65)cc4)c4cccc(-c5cccc6ccccc56)c4)cc3)cc2)cc1]

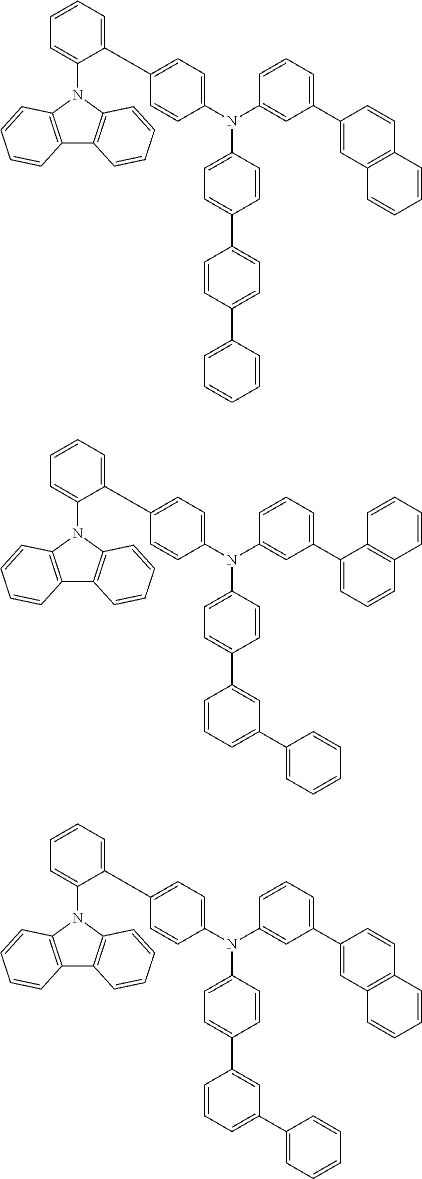 [SMILES: c1ccc(-c2ccc(-c3ccc(N(c4ccc(-c5ccccc5-n5c6ccccc6c6ccccc65)cc4)c4cccc(-c5ccc6ccccc6c5)c4)cc3)cc2)cc1 ; c1ccc(-c2cccc(-c3ccc(N(c4ccc(-c5ccccc5-n5c6ccccc6c6ccccc65)cc4)c4cccc(-c5cccc6ccccc56)c4)cc3)c2)cc1 ; c1ccc(-c2cccc(-c3ccc(N(c4ccc(-c5ccccc5-n5c6ccccc6c6ccccc65)cc4)c4cccc(-c5ccc6ccccc6c5)c4)cc3)c2)cc1]

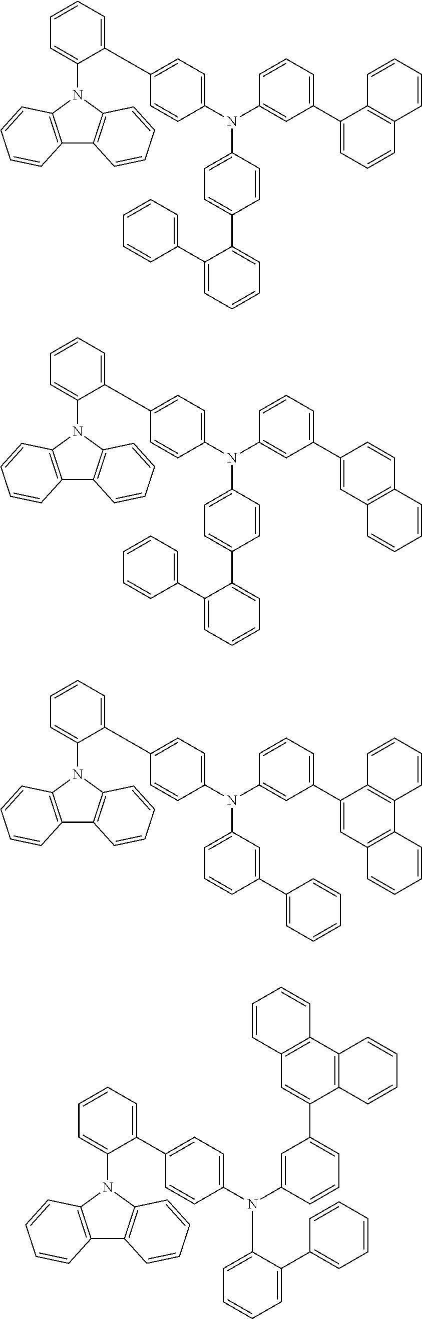 [SMILES: c1ccc(-c2ccccc2-c2ccc(N(c3ccc(-c4ccccc4-n4c5ccccc5c5ccccc54)cc3)c3cccc(-c4cccc5ccccc45)c3)cc2)cc1 ; c1ccc(-c2ccccc2-c2ccc(N(c3ccc(-c4ccccc4-n4c5ccccc5c5ccccc54)cc3)c3cccc(-c4ccc5ccccc5c4)c3)cc2)cc1 ; c1ccc(-c2cccc(N(c3ccc(-c4ccccc4-n4c5ccccc5c5ccccc54)cc3)c3cccc(-c4cc5ccccc5c5ccccc45)c3)c2)cc1 ; c1ccc(-c2ccccc2N(c2ccc(-c3ccccc3-n3c4ccccc4c4ccccc43)cc2)c2cccc(-c3cc4ccccc4c4ccccc34)c2)cc1]

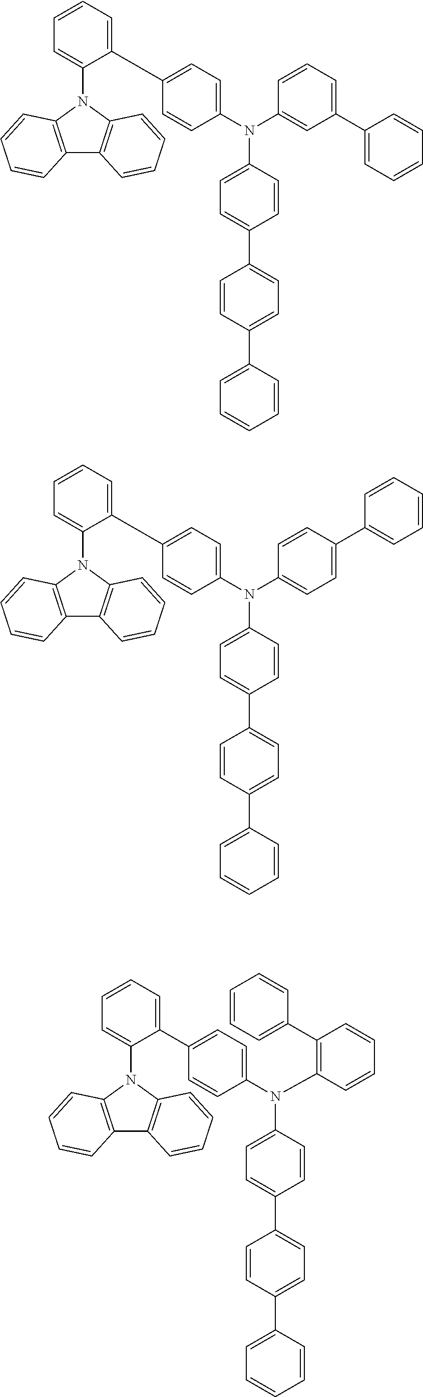 [SMILES: c1ccc(-c2ccc(-c3ccc(N(c4ccc(-c5ccccc5-n5c6ccccc6c6ccccc65)cc4)c4cccc(-c5ccccc5)c4)cc3)cc2)cc1 ; c1ccc(-c2ccc(-c3ccc(N(c4ccc(-c5ccccc5)cc4)c4ccc(-c5ccccc5-n5c6ccccc6c6ccccc65)cc4)cc3)cc2)cc1 ; c1ccc(-c2ccc(-c3ccc(N(c4ccc(-c5ccccc5-n5c6ccccc6c6ccccc65)cc4)c4ccccc4-c4ccccc4)cc3)cc2)cc1]

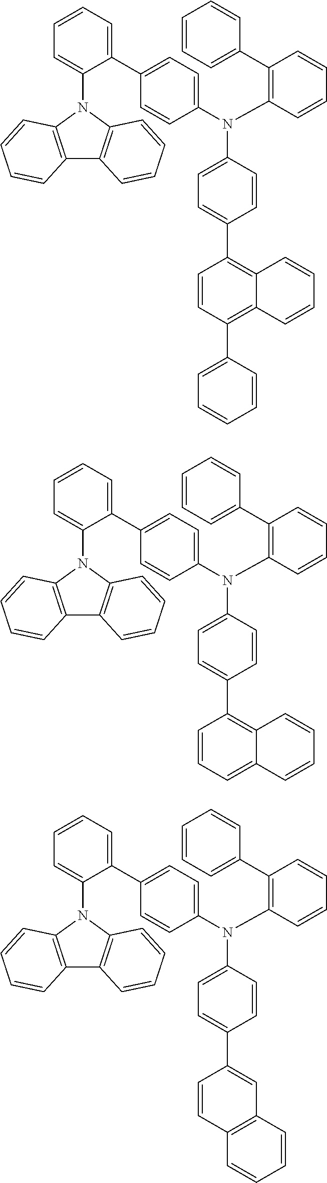

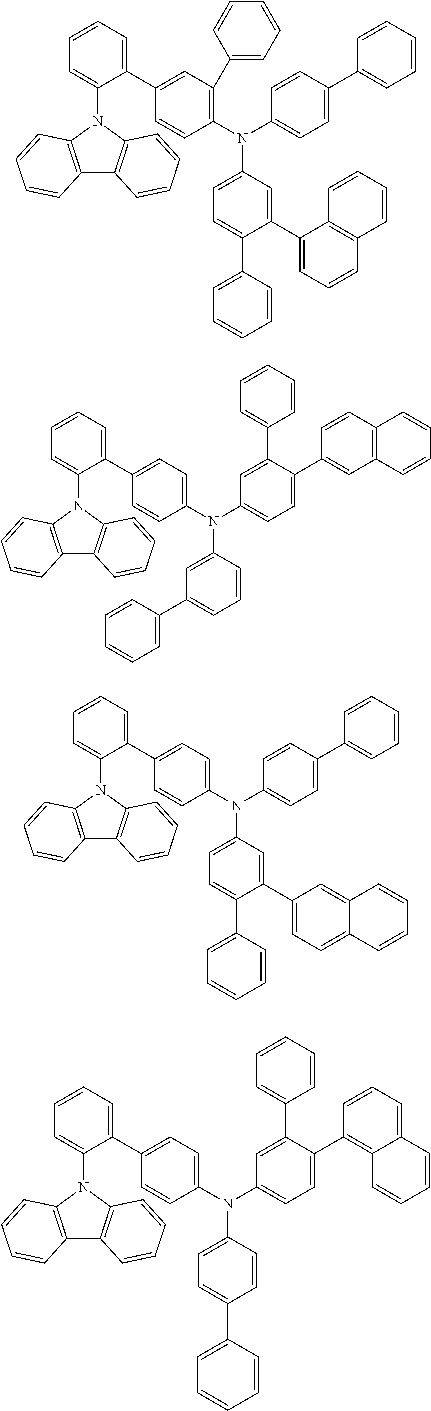

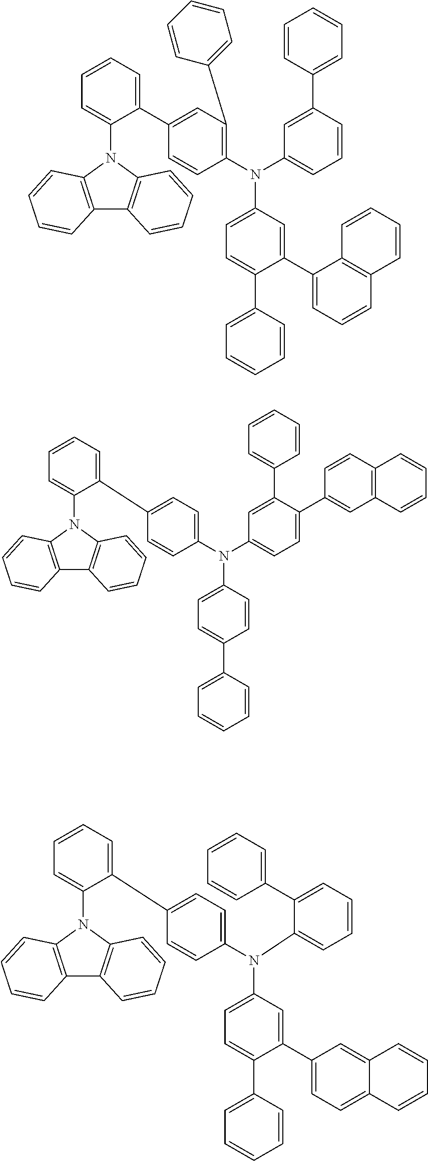

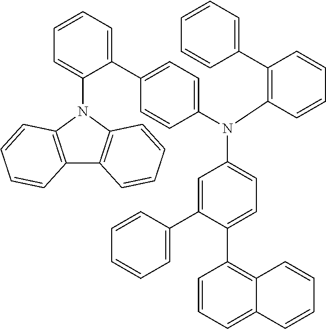 [SMILES: c1ccc(-c2cc(N(c3ccc(-c4ccccc4-n4c5ccccc5c5ccccc54)cc3)c3ccccc3-c3ccccc3)ccc2-c2cccc3ccccc23)cc1]

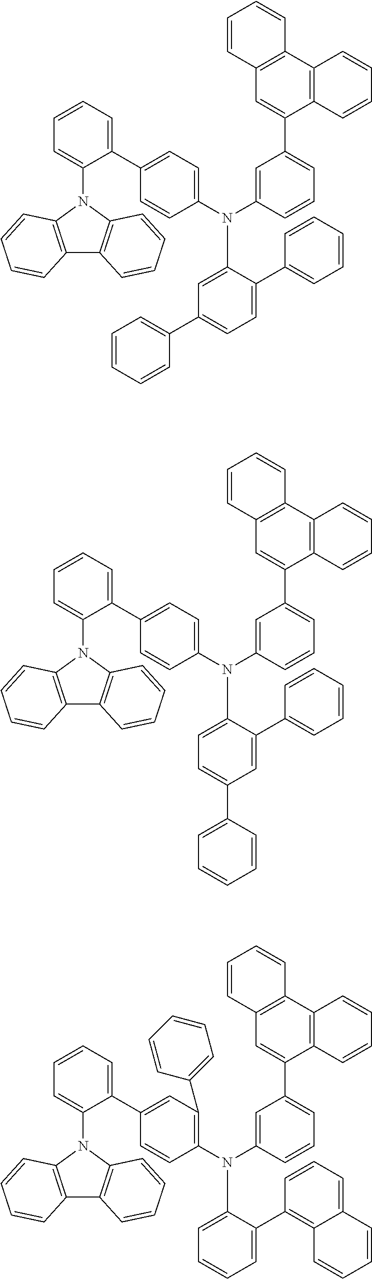 [SMILES: c1ccc(-c2ccc(-c3ccccc3)c(N(c3ccc(-c4ccccc4-n4c5ccccc5c5ccccc54)cc3)c3cccc(-c4cc5ccccc5c5ccccc45)c3)c2)cc1 ; c1ccc(-c2ccc(N(c3ccc(-c4ccccc4-n4c5ccccc5c5ccccc54)cc3)c3cccc(-c4cc5ccccc5c5ccccc45)c3)c(-c3ccccc3)c2)cc1 ; c1ccc(-c2cc(-c3ccccc3-n3c4ccccc4c4ccccc43)ccc2N(c2cccc(-c3cc4ccccc4c4ccccc34)c2)c2ccccc2-c2cccc3ccccc23)cc1]

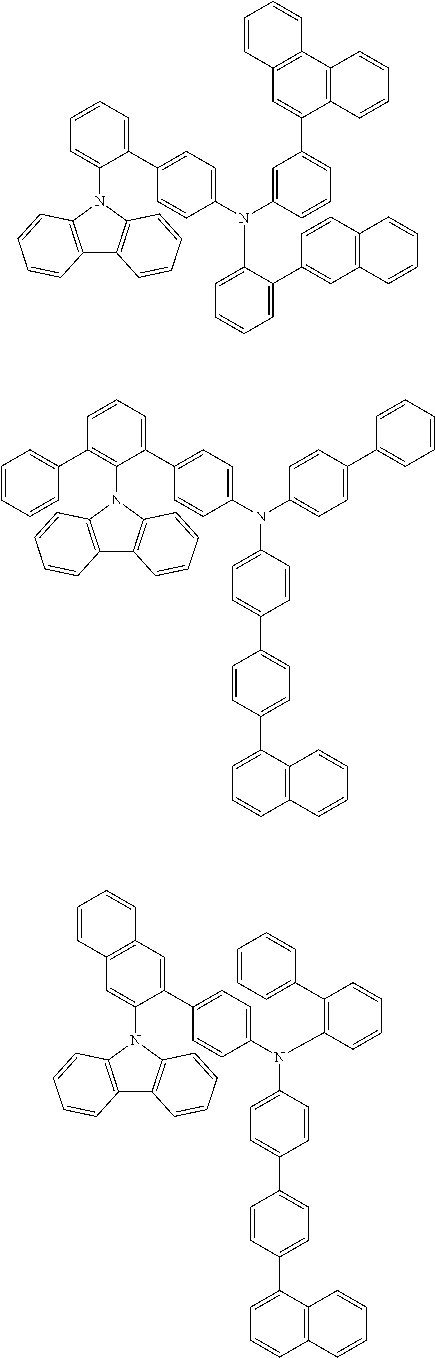 [SMILES: c1cc(-c2cc3ccccc3c3ccccc23)cc(N(c2ccc(-c3ccccc3-n3c4ccccc4c4ccccc43)cc2)c2ccccc2-c2ccc3ccccc3c2)c1 ; c1ccc(-c2ccc(N(c3ccc(-c4ccc(-c5cccc6ccccc56)cc4)cc3)c3ccc(-c4cccc(-c5ccccc5)c4-n4c5ccccc5c5ccccc54)cc3)cc2)cc1 ; c1ccc(-c2ccccc2N(c2ccc(-c3ccc(-c4cccc5ccccc45)cc3)cc2)c2ccc(-c3cc4ccccc4cc3-n3c4ccccc4c4ccccc43)cc2)cc1]

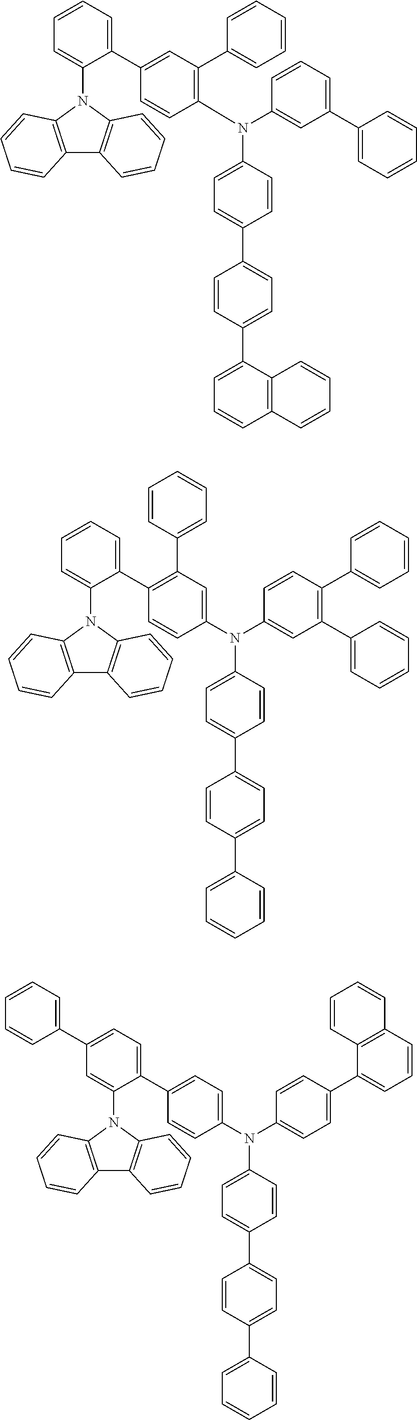 [SMILES: c1ccc(-c2cccc(N(c3ccc(-c4ccc(-c5cccc6ccccc56)cc4)cc3)c3ccc(-c4ccccc4-n4c5ccccc5c5ccccc54)cc3-c3ccccc3)c2)cc1 ; c1ccc(-c2ccc(-c3ccc(N(c4ccc(-c5ccccc5)c(-c5ccccc5)c4)c4ccc(-c5ccccc5-n5c6ccccc6c6ccccc65)c(-c5ccccc5)c4)cc3)cc2)cc1 ; c1ccc(-c2ccc(-c3ccc(N(c4ccc(-c5ccc(-c6ccccc6)cc5-n5c6ccccc6c6ccccc65)cc4)c4ccc(-c5cccc6ccccc56)cc4)cc3)cc2)cc1]

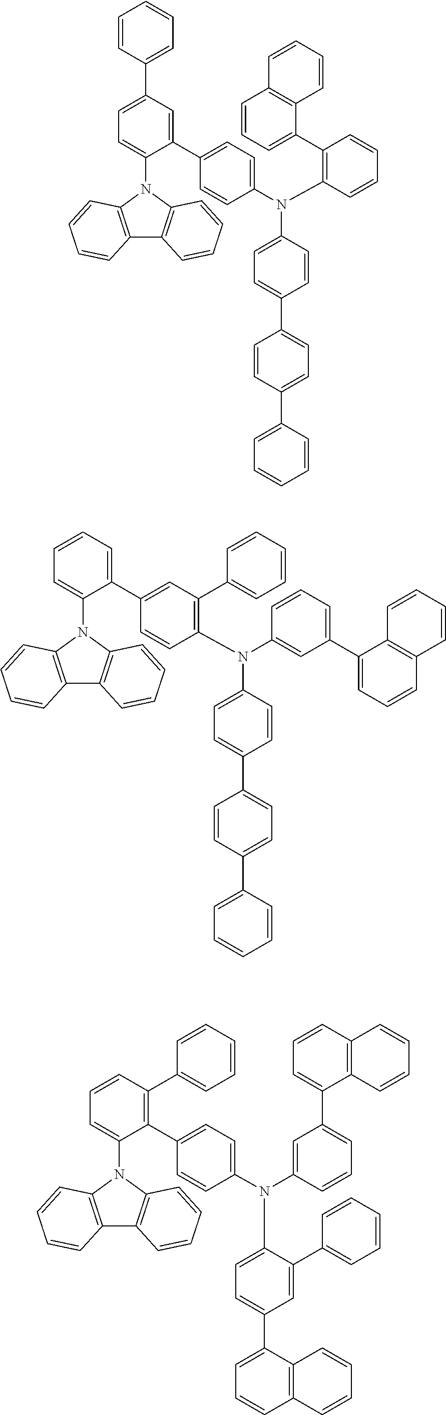 [SMILES: c1ccc(-c2ccc(-c3ccc(N(c4ccc(-c5cc(-c6ccccc6)ccc5-n5c6ccccc6c6ccccc65)cc4)c4ccccc4-c4cccc5ccccc45)cc3)cc2)cc1 ; c1ccc(-c2ccc(-c3ccc(N(c4cccc(-c5cccc6ccccc56)c4)c4ccc(-c5ccccc5-n5c6ccccc6c6ccccc65)cc4-c4ccccc4)cc3)cc2)cc1 ; c1ccc(-c2cc(-c3cccc4ccccc34)ccc2N(c2ccc(-c3c(-c4ccccc4)cccc3-n3c4ccccc4c4ccccc43)cc2)c2cccc(-c3cccc4ccccc34)c2)cc1]

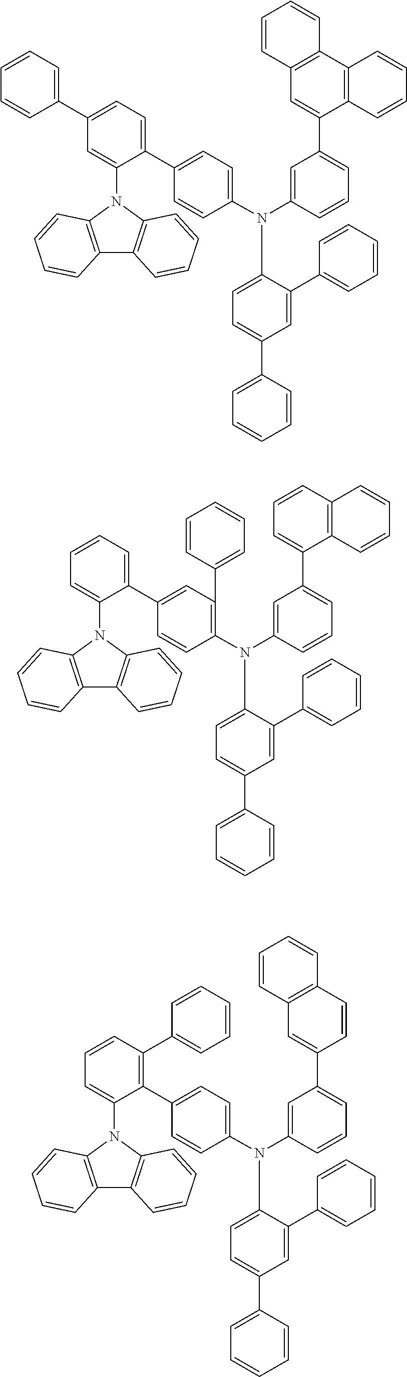 [SMILES: c1ccc(-c2ccc(N(c3ccc(-c4ccc(-c5ccccc5)cc4-n4c5ccccc5c5ccccc54)cc3)c3cccc(-c4cc5ccccc5c5ccccc45)c3)c(-c3ccccc3)c2)cc1 ; c1ccc(-c2ccc(N(c3cccc(-c4cccc5ccccc45)c3)c3ccc(-c4ccccc4-n4c5ccccc5c5ccccc54)cc3-c3ccccc3)c(-c3ccccc3)c2)cc1 ; c1ccc(-c2ccc(N(c3ccc(-c4c(-c5ccccc5)cccc4-n4c5ccccc5c5ccccc54)cc3)c3cccc(-c4ccc5ccccc5c4)c3)c(-c3ccccc3)c2)cc1]

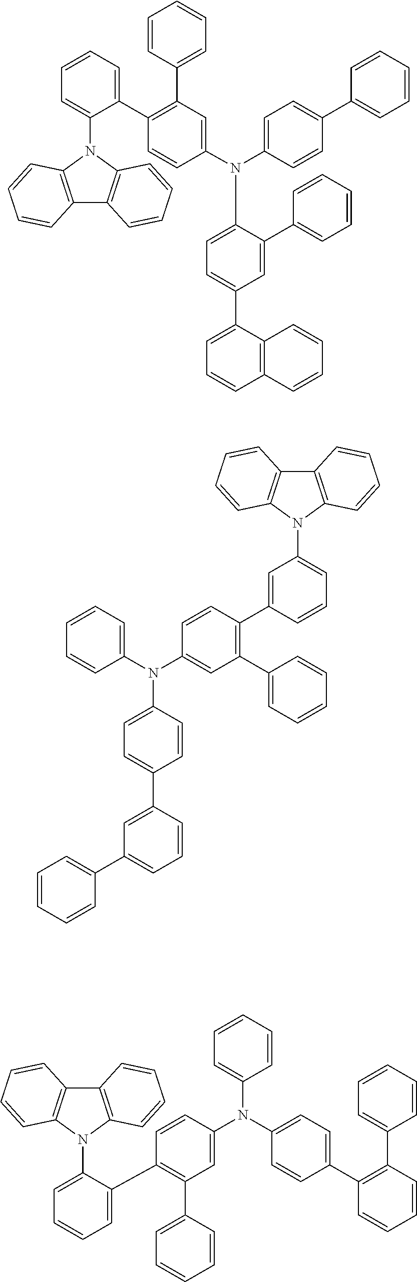 [SMILES: c1ccc(-c2ccc(N(c3ccc(-c4ccccc4-n4c5ccccc5c5ccccc54)c(-c4ccccc4)c3)c3ccc(-c4cccc5ccccc45)cc3-c3ccccc3)cc2)cc1 ; c1ccc(-c2cccc(-c3ccc(N(c4ccccc4)c4ccc(-c5cccc(-n6c7ccccc7c7ccccc76)c5)c(-c5ccccc5)c4)cc3)c2)cc1 ; c1ccc(-c2ccccc2-c2ccc(N(c3ccccc3)c3ccc(-c4ccccc4-n4c5ccccc5c5ccccc54)c(-c4ccccc4)c3)cc2)cc1]

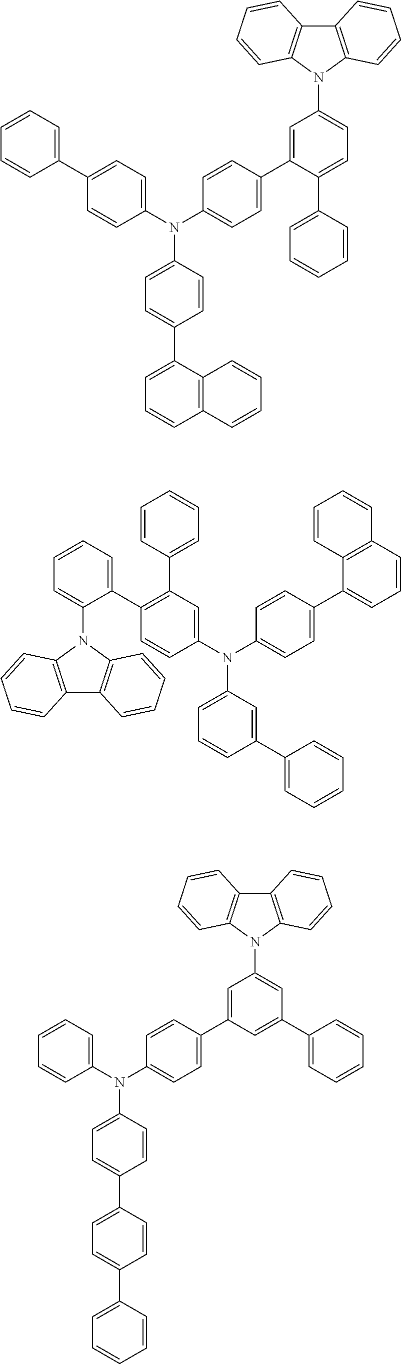 [SMILES: c1ccc(-c2ccc(N(c3ccc(-c4cc(-n5c6ccccc6c6ccccc65)ccc4-c4ccccc4)cc3)c3ccc(-c4cccc5ccccc45)cc3)cc2)cc1 ; c1ccc(-c2cccc(N(c3ccc(-c4cccc5ccccc45)cc3)c3ccc(-c4ccccc4-n4c5ccccc5c5ccccc54)c(-c4ccccc4)c3)c2)cc1 ; c1ccc(-c2ccc(-c3ccc(N(c4ccccc4)c4ccc(-c5cc(-c6ccccc6)cc(-n6c7ccccc7c7ccccc76)c5)cc4)cc3)cc2)cc1]

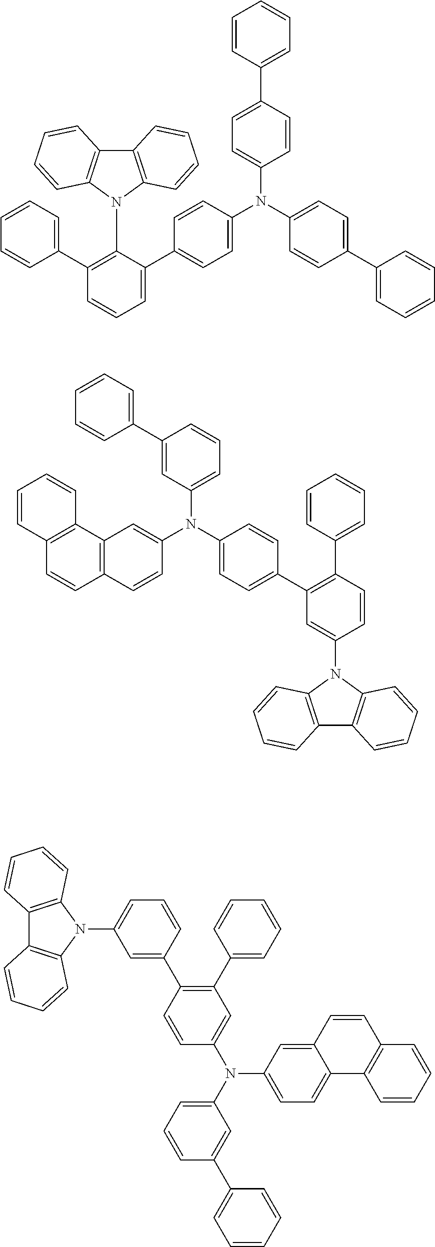 [SMILES: c1ccc(-c2ccc(N(c3ccc(-c4ccccc4)cc3)c3ccc(-c4cccc(-c5ccccc5)c4-n4c5ccccc5c5ccccc54)cc3)cc2)cc1 ; c1ccc(-c2cccc(N(c3ccc(-c4cc(-n5c6ccccc6c6ccccc65)ccc4-c4ccccc4)cc3)c3ccc4ccc5ccccc5c4c3)c2)cc1 ; c1ccc(-c2cccc(N(c3ccc(-c4cccc(-n5c6ccccc6c6ccccc65)c4)c(-c4ccccc4)c3)c3ccc4c(ccc5ccccc54)c3)c2)cc1]

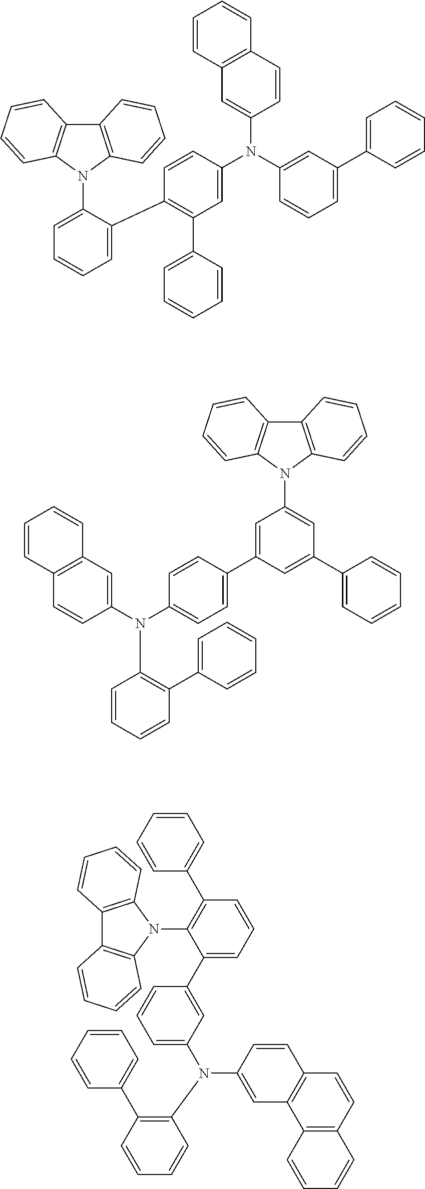 [SMILES: c1ccc(-c2cccc(N(c3ccc(-c4ccccc4-n4c5ccccc5c5ccccc54)c(-c4ccccc4)c3)c3ccc4ccccc4c3)c2)cc1 ; c1ccc(-c2cc(-c3ccc(N(c4ccc5ccccc5c4)c4ccccc4-c4ccccc4)cc3)cc(-n3c4ccccc4c4ccccc43)c2)cc1 ; c1ccc(-c2ccccc2N(c2cccc(-c3cccc(-c4ccccc4)c3-n3c4ccccc4c4ccccc43)c2)c2ccc3ccc4ccccc4c3c2)cc1]

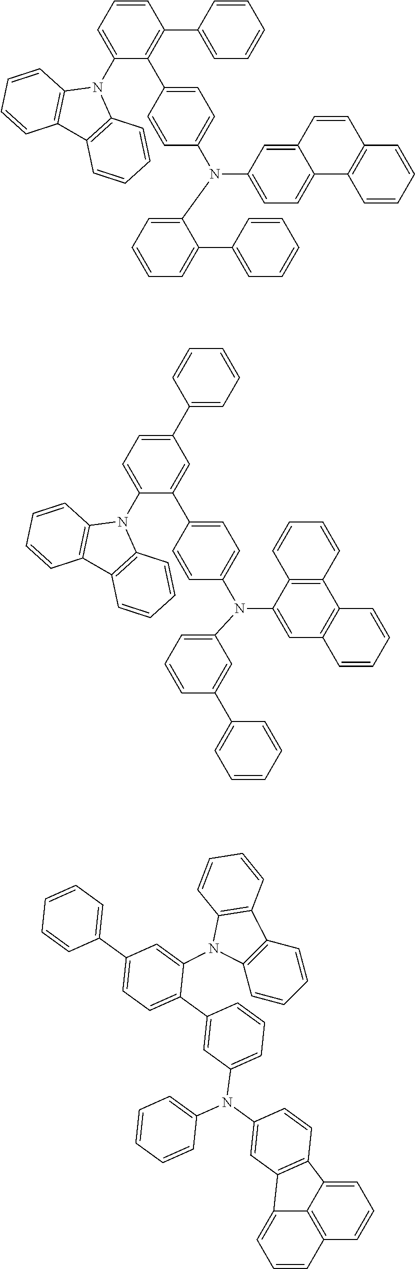 [SMILES: c1ccc(-c2ccccc2N(c2ccc(-c3c(-c4ccccc4)cccc3-n3c4ccccc4c4ccccc43)cc2)c2ccc3c(ccc4ccccc43)c2)cc1 ; c1ccc(-c2cccc(N(c3ccc(-c4cc(-c5ccccc5)ccc4-n4c5ccccc5c5ccccc54)cc3)c3cc4ccccc4c4ccccc34)c2)cc1 ; c1ccc(-c2ccc(-c3cccc(N(c4ccccc4)c4ccc5c(c4)-c4cccc6cccc-5c46)c3)c(-n3c4ccccc4c4ccccc43)c2)cc1]

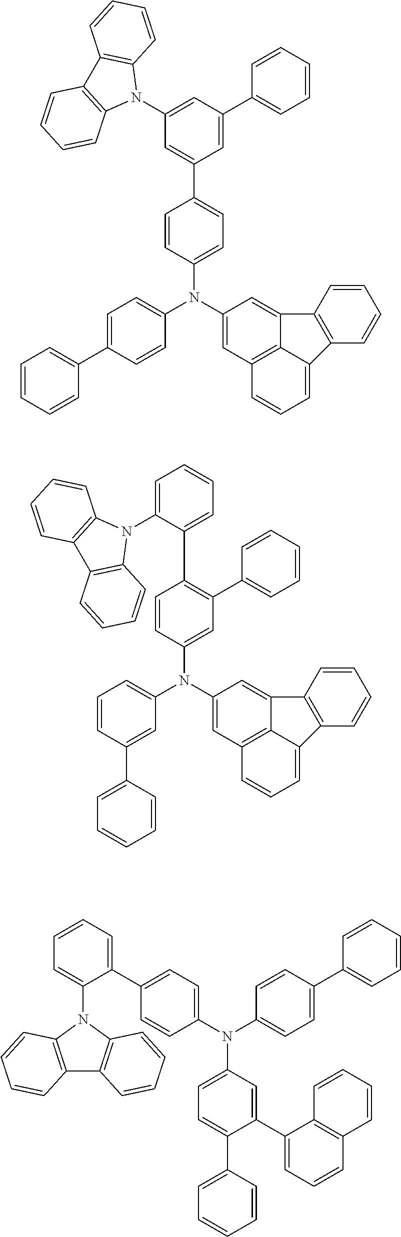 [SMILES: c1ccc(-c2ccc(N(c3ccc(-c4cc(-c5ccccc5)cc(-n5c6ccccc6c6ccccc65)c4)cc3)c3cc4c5c(cccc5c3)-c3ccccc3-4)cc2)cc1 ; c1ccc(-c2cccc(N(c3ccc(-c4ccccc4-n4c5ccccc5c5ccccc54)c(-c4ccccc4)c3)c3cc4c5c(cccc5c3)-c3ccccc3-4)c2)cc1 ; c1ccc(-c2ccc(N(c3ccc(-c4ccccc4-n4c5ccccc5c5ccccc54)cc3)c3ccc(-c4ccccc4)c(-c4cccc5ccccc45)c3)cc2)cc1]

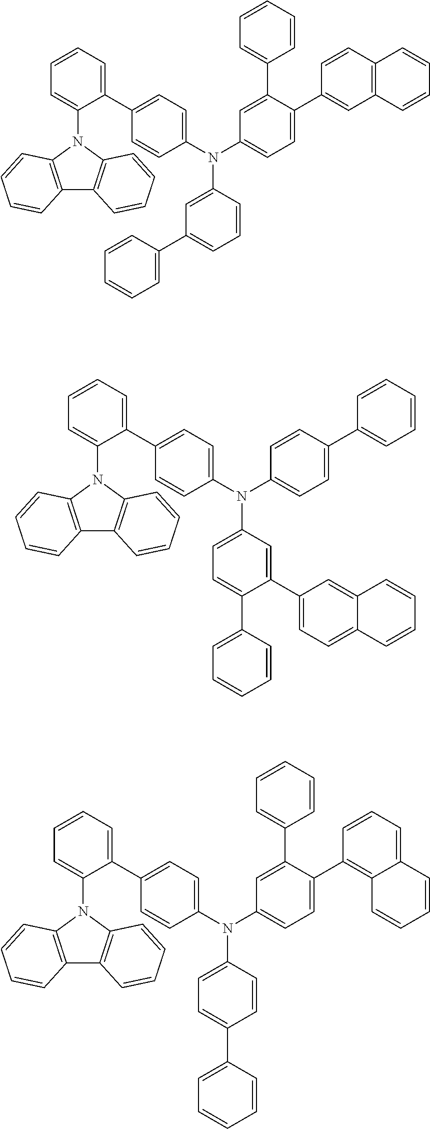 [SMILES: c1ccc(-c2cccc(N(c3ccc(-c4ccccc4-n4c5ccccc5c5ccccc54)cc3)c3ccc(-c4ccc5ccccc5c4)c(-c4ccccc4)c3)c2)cc1 ; c1ccc(-c2ccc(N(c3ccc(-c4ccccc4-n4c5ccccc5c5ccccc54)cc3)c3ccc(-c4ccccc4)c(-c4ccc5ccccc5c4)c3)cc2)cc1 ; c1ccc(-c2ccc(N(c3ccc(-c4ccccc4-n4c5ccccc5c5ccccc54)cc3)c3ccc(-c4cccc5ccccc45)c(-c4ccccc4)c3)cc2)cc1]

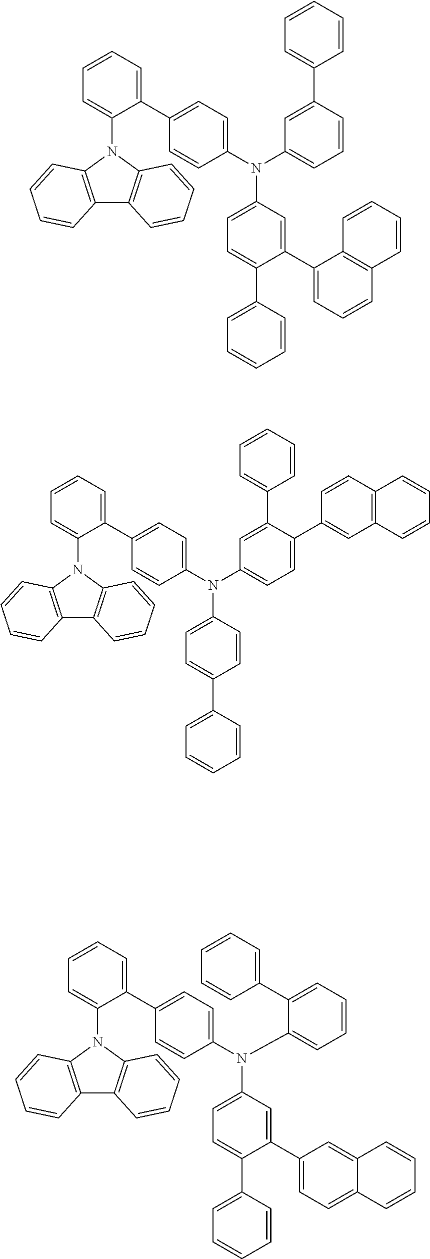 [SMILES: c1ccc(-c2cccc(N(c3ccc(-c4ccccc4-n4c5ccccc5c5ccccc54)cc3)c3ccc(-c4ccccc4)c(-c4cccc5ccccc45)c3)c2)cc1 ; c1ccc(-c2ccc(N(c3ccc(-c4ccccc4-n4c5ccccc5c5ccccc54)cc3)c3ccc(-c4ccc5ccccc5c4)c(-c4ccccc4)c3)cc2)cc1 ; c1ccc(-c2ccc(N(c3ccc(-c4ccccc4-n4c5ccccc5c5ccccc54)cc3)c3ccccc3-c3ccccc3)cc2-c2ccc3ccccc3c2)cc1]

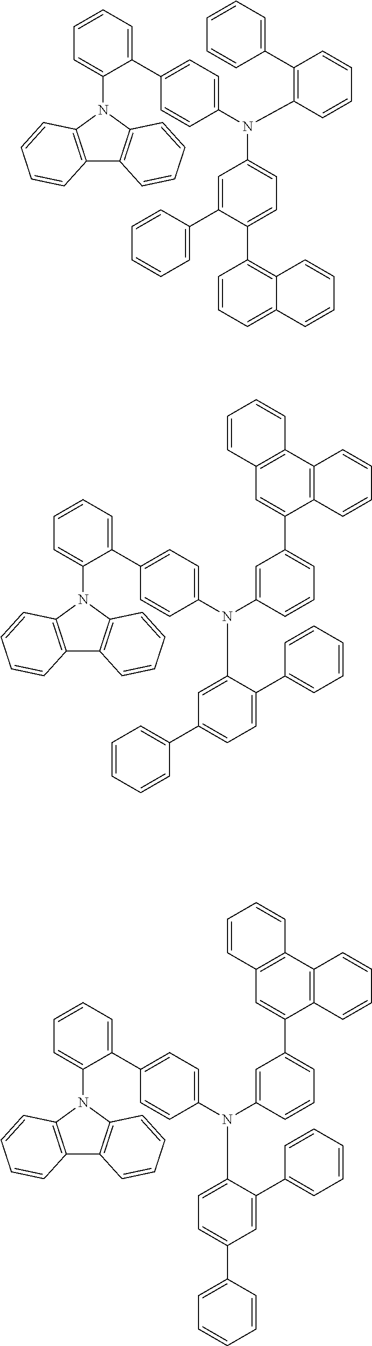 [SMILES: c1ccc(-c2cc(N(c3ccc(-c4ccccc4-n4c5ccccc5c5ccccc54)cc3)c3ccccc3-c3ccccc3)ccc2-c2cccc3ccccc23)cc1 ; c1ccc(-c2ccc(-c3ccccc3)c(N(c3ccc(-c4ccccc4-n4c5ccccc5c5ccccc54)cc3)c3cccc(-c4cc5ccccc5c5ccccc45)c3)c2)cc1 ; c1ccc(-c2ccc(N(c3ccc(-c4ccccc4-n4c5ccccc5c5ccccc54)cc3)c3cccc(-c4cc5ccccc5c5ccccc45)c3)c(-c3ccccc3)c2)cc1]

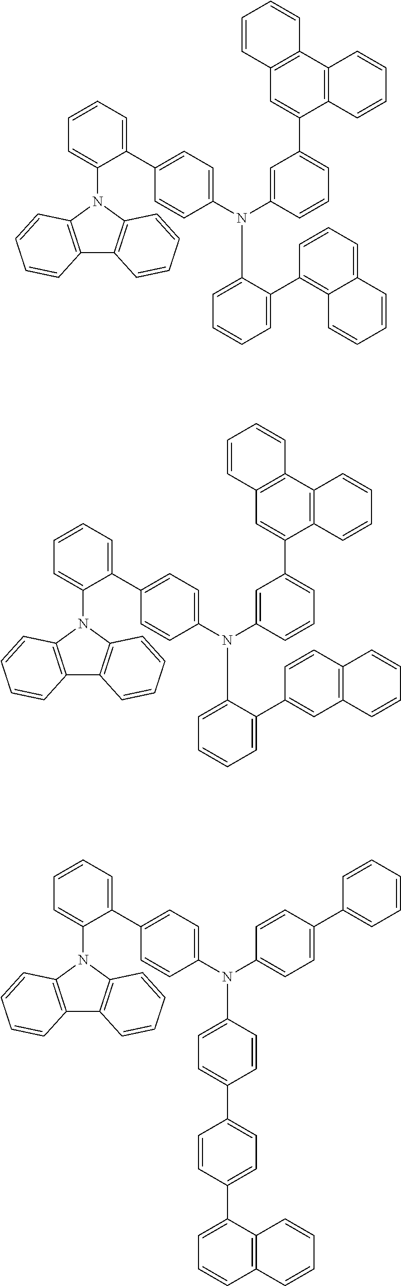 [SMILES: c1cc(-c2cc3ccccc3c3ccccc23)cc(N(c2ccc(-c3ccccc3-n3c4ccccc4c4ccccc43)cc2)c2ccccc2-c2cccc3ccccc23)c1 ; c1cc(-c2cc3ccccc3c3ccccc23)cc(N(c2ccc(-c3ccccc3-n3c4ccccc4c4ccccc43)cc2)c2ccccc2-c2ccc3ccccc3c2)c1 ; c1ccc(-c2ccc(N(c3ccc(-c4ccc(-c5cccc6ccccc56)cc4)cc3)c3ccc(-c4ccccc4-n4c5ccccc5c5ccccc54)cc3)cc2)cc1]

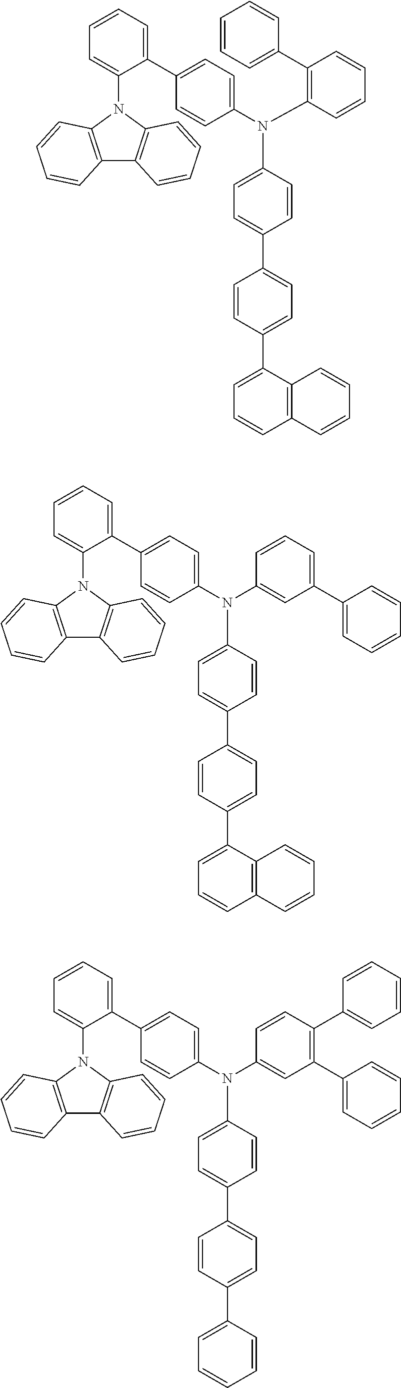 [SMILES: c1ccc(-c2ccccc2N(c2ccc(-c3ccc(-c4cccc5ccccc45)cc3)cc2)c2ccc(-c3ccccc3-n3c4ccccc4c4ccccc43)cc2)cc1 ; c1ccc(-c2cccc(N(c3ccc(-c4ccc(-c5cccc6ccccc56)cc4)cc3)c3ccc(-c4ccccc4-n4c5ccccc5c5ccccc54)cc3)c2)cc1 ; c1ccc(-c2ccc(-c3ccc(N(c4ccc(-c5ccccc5-n5c6ccccc6c6ccccc65)cc4)c4ccc(-c5ccccc5)c(-c5ccccc5)c4)cc3)cc2)cc1]

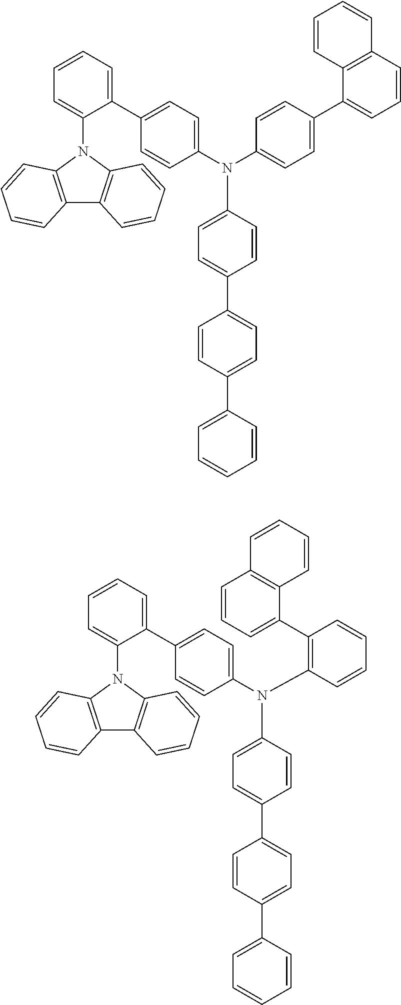 [SMILES: c1ccc(-c2ccc(-c3ccc(N(c4ccc(-c5ccccc5-n5c6ccccc6c6ccccc65)cc4)c4ccc(-c5cccc6ccccc56)cc4)cc3)cc2)cc1 ; c1ccc(-c2ccc(-c3ccc(N(c4ccc(-c5ccccc5-n5c6ccccc6c6ccccc65)cc4)c4ccccc4-c4cccc5ccccc45)cc3)cc2)cc1]

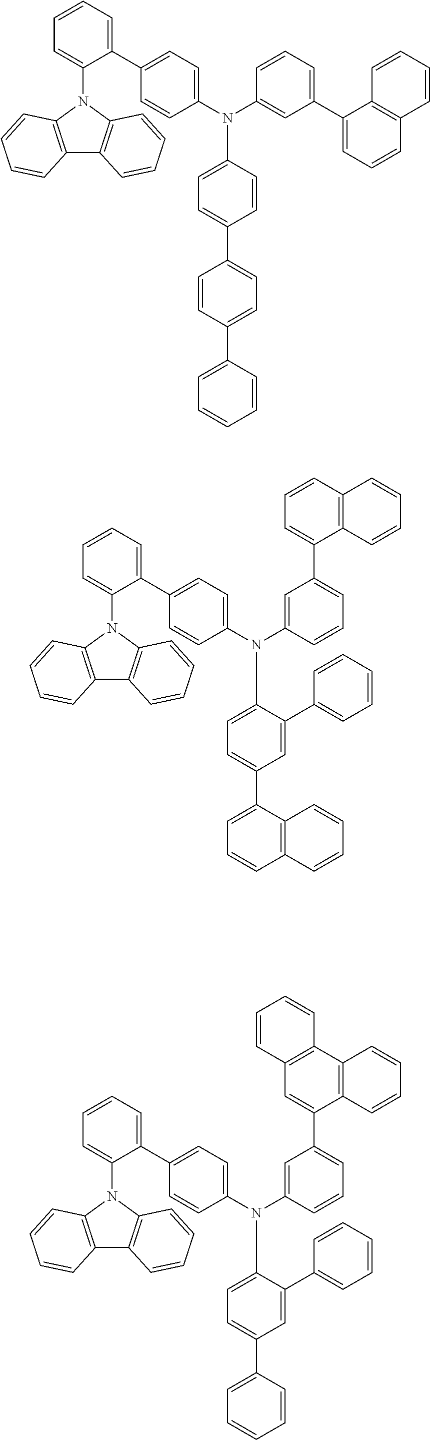 [SMILES: c1ccc(-c2ccc(-c3ccc(N(c4ccc(-c5ccccc5-n5c6ccccc6c6ccccc65)cc4)c4cccc(-c5cccc6ccccc56)c4)cc3)cc2)cc1 ; c1ccc(-c2cc(-c3cccc4ccccc34)ccc2N(c2ccc(-c3ccccc3-n3c4ccccc4c4ccccc43)cc2)c2cccc(-c3cccc4ccccc34)c2)cc1 ; c1ccc(-c2ccc(N(c3ccc(-c4ccccc4-n4c5ccccc5c5ccccc54)cc3)c3cccc(-c4cc5ccccc5c5ccccc45)c3)c(-c3ccccc3)c2)cc1]

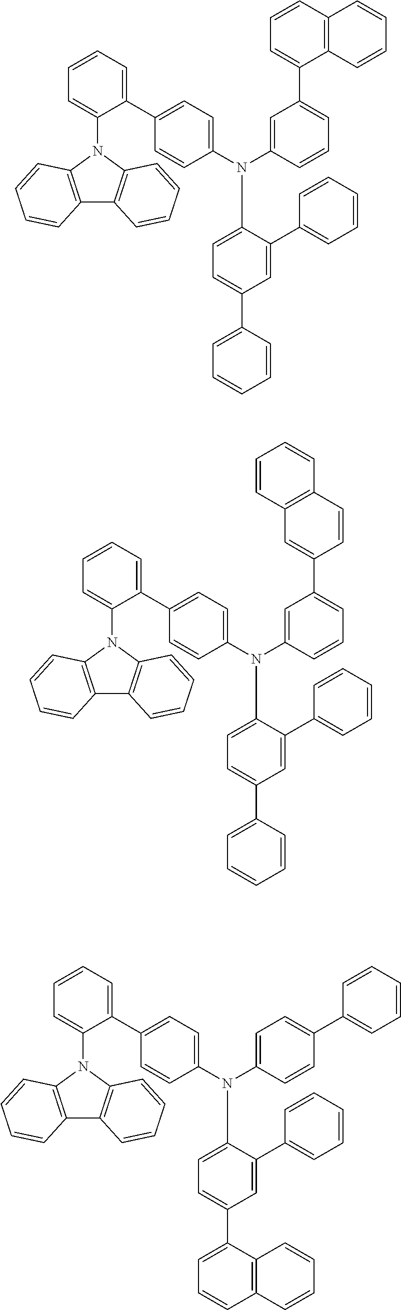 [SMILES: c1ccc(-c2ccc(N(c3ccc(-c4ccccc4-n4c5ccccc5c5ccccc54)cc3)c3cccc(-c4cccc5ccccc45)c3)c(-c3ccccc3)c2)cc1 ; c1ccc(-c2ccc(N(c3ccc(-c4ccccc4-n4c5ccccc5c5ccccc54)cc3)c3cccc(-c4ccc5ccccc5c4)c3)c(-c3ccccc3)c2)cc1 ; c1ccc(-c2ccc(N(c3ccc(-c4ccccc4-n4c5ccccc5c5ccccc54)cc3)c3ccc(-c4cccc5ccccc45)cc3-c3ccccc3)cc2)cc1]

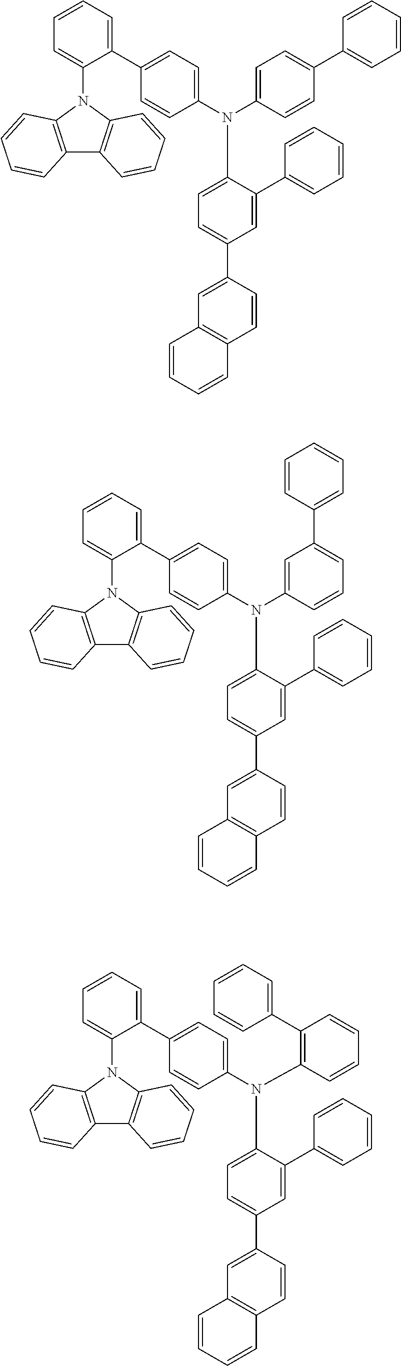 [SMILES: c1ccc(-c2ccc(N(c3ccc(-c4ccccc4-n4c5ccccc5c5ccccc54)cc3)c3ccc(-c4ccc5ccccc5c4)cc3-c3ccccc3)cc2)cc1 ; c1ccc(-c2cccc(N(c3ccc(-c4ccccc4-n4c5ccccc5c5ccccc54)cc3)c3ccc(-c4ccc5ccccc5c4)cc3-c3ccccc3)c2)cc1 ; c1ccc(-c2ccccc2N(c2ccc(-c3ccccc3-n3c4ccccc4c4ccccc43)cc2)c2ccc(-c3ccc4ccccc4c3)cc2-c2ccccc2)cc1]

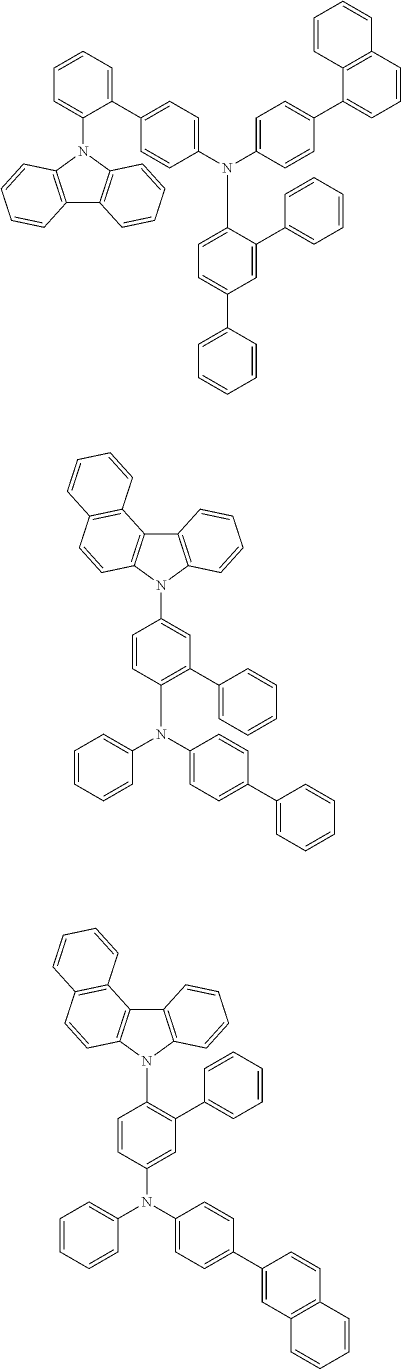 [SMILES: c1ccc(-c2ccc(N(c3ccc(-c4ccccc4-n4c5ccccc5c5ccccc54)cc3)c3ccc(-c4cccc5ccccc45)cc3)c(-c3ccccc3)c2)cc1 ; c1ccc(-c2ccc(N(c3ccccc3)c3ccc(-n4c5ccccc5c5c6ccccc6ccc54)cc3-c3ccccc3)cc2)cc1 ; c1ccc(-c2cc(N(c3ccccc3)c3ccc(-c4ccc5ccccc5c4)cc3)ccc2-n2c3ccccc3c3c4ccccc4ccc32)cc1]

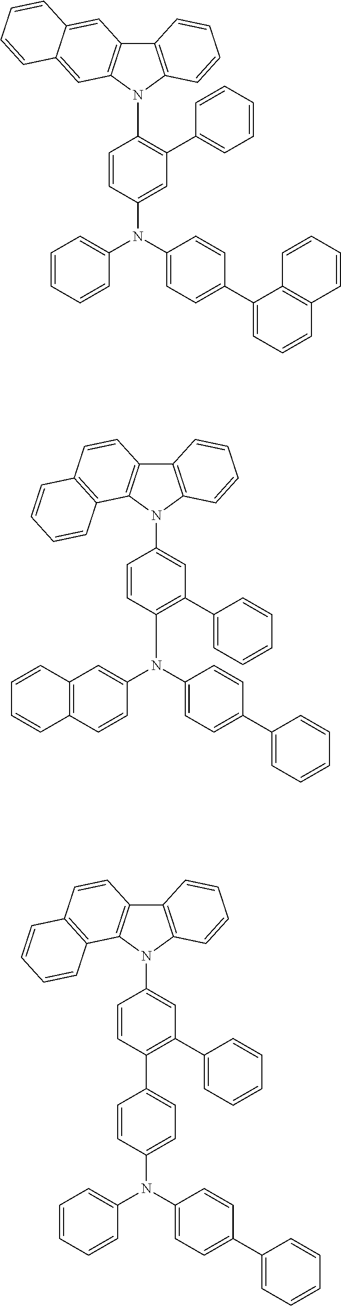 [SMILES: c1ccc(-c2cc(N(c3ccccc3)c3ccc(-c4cccc5ccccc45)cc3)ccc2-n2c3ccccc3c3cc4ccccc4cc32)cc1 ; c1ccc(-c2ccc(N(c3ccc4ccccc4c3)c3ccc(-n4c5ccccc5c5ccc6ccccc6c54)cc3-c3ccccc3)cc2)cc1 ; c1ccc(-c2ccc(N(c3ccccc3)c3ccc(-c4ccc(-n5c6ccccc6c6ccc7ccccc7c65)cc4-c4ccccc4)cc3)cc2)cc1]

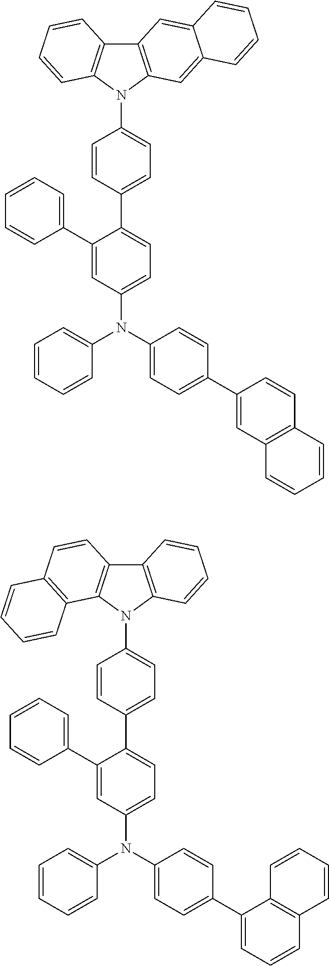 [SMILES: c1ccc(-c2cc(N(c3ccccc3)c3ccc(-c4ccc5ccccc5c4)cc3)ccc2-c2ccc(-n3c4ccccc4c4cc5ccccc5cc43)cc2)cc1 ; c1ccc(-c2cc(N(c3ccccc3)c3ccc(-c4cccc5ccccc45)cc3)ccc2-c2ccc(-n3c4ccccc4c4ccc5ccccc5c43)cc2)cc1]

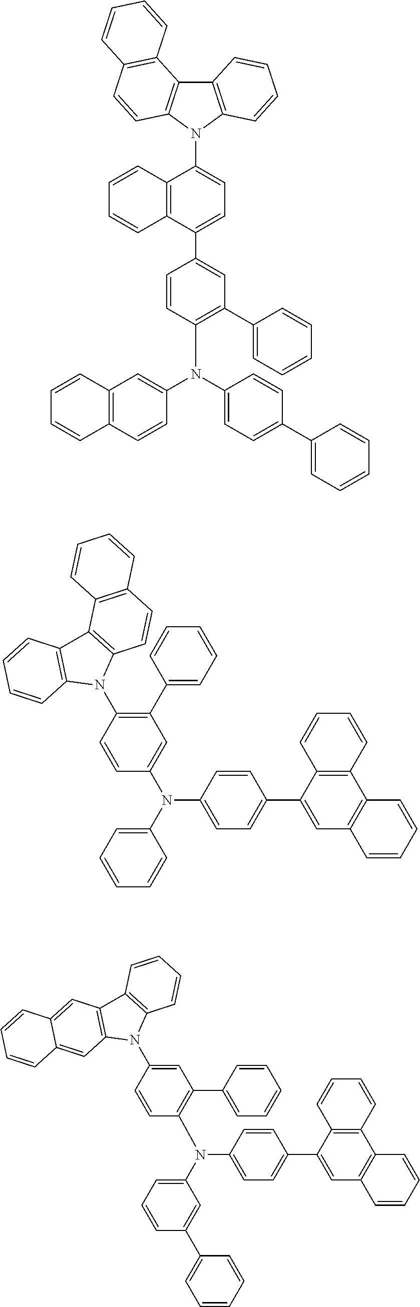 [SMILES: c1ccc(-c2ccc(N(c3ccc4ccccc4c3)c3ccc(-c4ccc(-n5c6ccccc6c6c7ccccc7ccc65)c5ccccc45)cc3-c3ccccc3)cc2)cc1 ; c1ccc(-c2cc(N(c3ccccc3)c3ccc(-c4cc5ccccc5c5ccccc45)cc3)ccc2-n2c3ccccc3c3c4ccccc4ccc32)cc1 ; c1ccc(-c2cccc(N(c3ccc(-c4cc5ccccc5c5ccccc45)cc3)c3ccc(-n4c5ccccc5c5cc6ccccc6cc54)cc3-c3ccccc3)c2)cc1]

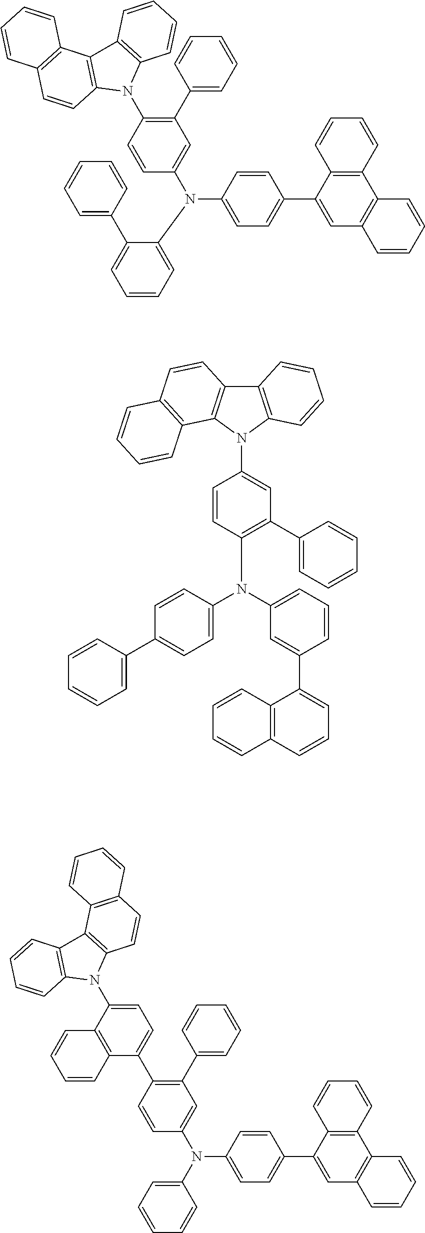 [SMILES: c1ccc(-c2ccccc2N(c2ccc(-c3cc4ccccc4c4ccccc34)cc2)c2ccc(-n3c4ccccc4c4c5ccccc5ccc43)c(-c3ccccc3)c2)cc1 ; c1ccc(-c2ccc(N(c3cccc(-c4cccc5ccccc45)c3)c3ccc(-n4c5ccccc5c5ccc6ccccc6c54)cc3-c3ccccc3)cc2)cc1 ; c1ccc(-c2cc(N(c3ccccc3)c3ccc(-c4cc5ccccc5c5ccccc45)cc3)ccc2-c2ccc(-n3c4ccccc4c4c5ccccc5ccc43)c3ccccc23)cc1]

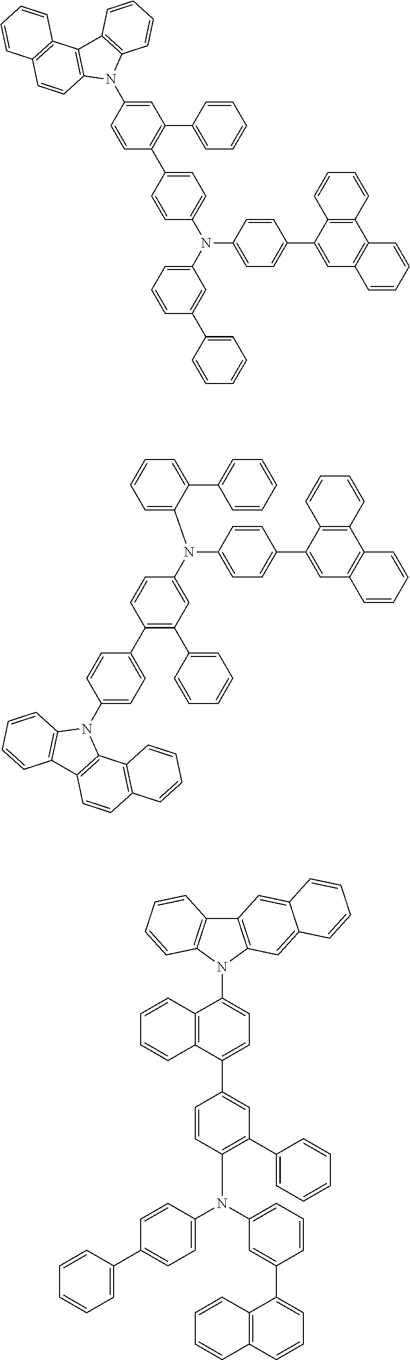 [SMILES: c1ccc(-c2cccc(N(c3ccc(-c4ccc(-n5c6ccccc6c6c7ccccc7ccc65)cc4-c4ccccc4)cc3)c3ccc(-c4cc5ccccc5c5ccccc45)cc3)c2)cc1 ; c1ccc(-c2cc(N(c3ccc(-c4cc5ccccc5c5ccccc45)cc3)c3ccccc3-c3ccccc3)ccc2-c2ccc(-n3c4ccccc4c4ccc5ccccc5c43)cc2)cc1 ; c1ccc(-c2ccc(N(c3cccc(-c4cccc5ccccc45)c3)c3ccc(-c4ccc(-n5c6ccccc6c6cc7ccccc7cc65)c5ccccc45)cc3-c3ccccc3)cc2)cc1]

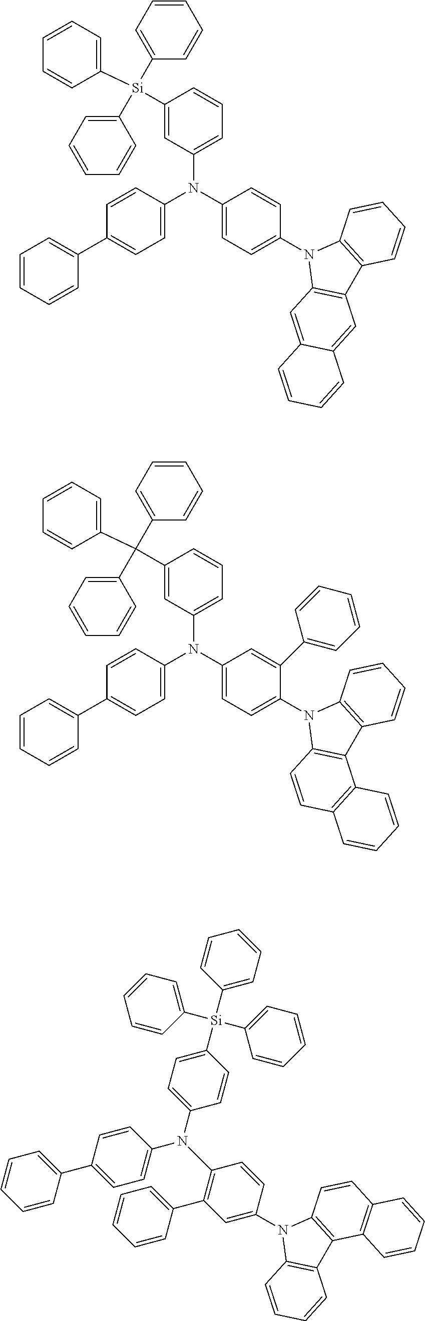 [SMILES: c1ccc(-c2ccc(N(c3ccc(-n4c5ccccc5c5cc6ccccc6cc54)cc3)c3cccc([Si](c4ccccc4)(c4ccccc4)c4ccccc4)c3)cc2)cc1 ; c1ccc(-c2ccc(N(c3cccc(C(c4ccccc4)(c4ccccc4)c4ccccc4)c3)c3ccc(-n4c5ccccc5c5c6ccccc6ccc54)c(-c4ccccc4)c3)cc2)cc1 ; c1ccc(-c2ccc(N(c3ccc([Si](c4ccccc4)(c4ccccc4)c4ccccc4)cc3)c3ccc(-n4c5ccccc5c5c6ccccc6ccc54)cc3-c3ccccc3)cc2)cc1]

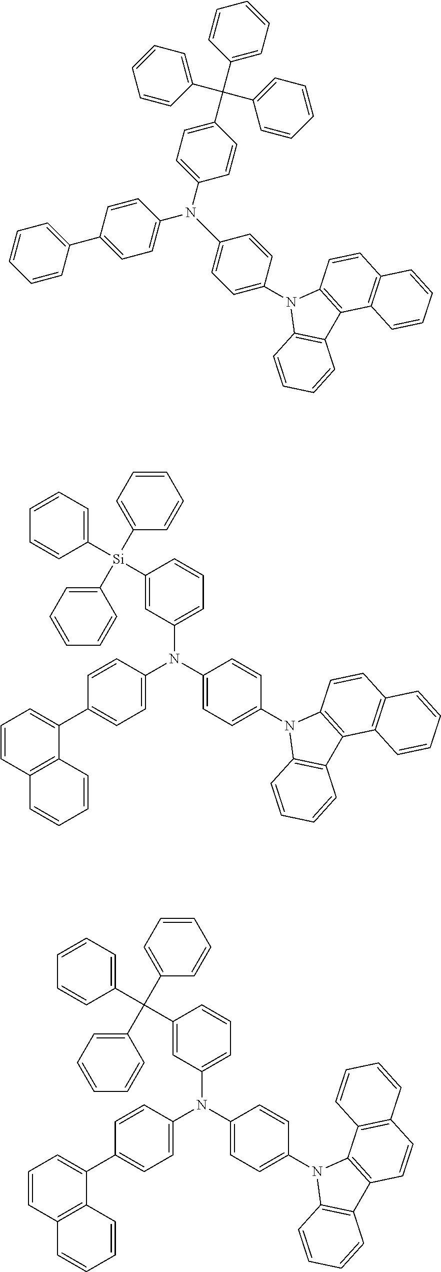 [SMILES: c1ccc(-c2ccc(N(c3ccc(-n4c5ccccc5c5c6ccccc6ccc54)cc3)c3ccc(C(c4ccccc4)(c4ccccc4)c4ccccc4)cc3)cc2)cc1 ; c1ccc([Si](c2ccccc2)(c2ccccc2)c2cccc(N(c3ccc(-c4cccc5ccccc45)cc3)c3ccc(-n4c5ccccc5c5c6ccccc6ccc54)cc3)c2)cc1 ; c1ccc(C(c2ccccc2)(c2ccccc2)c2cccc(N(c3ccc(-c4cccc5ccccc45)cc3)c3ccc(-n4c5ccccc5c5ccc6ccccc6c54)cc3)c2)cc1]

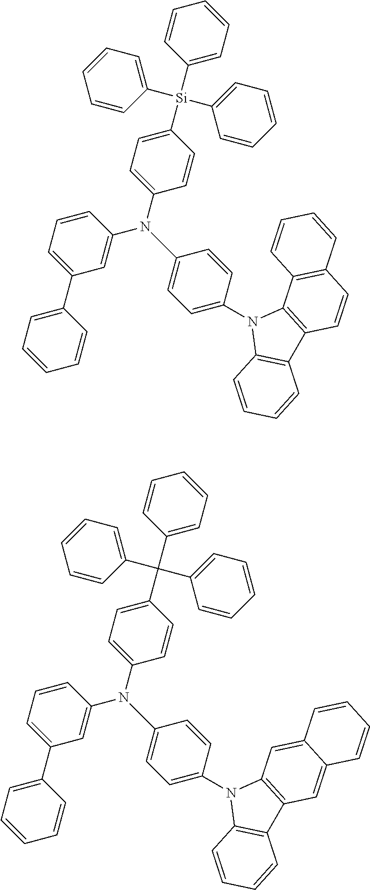 [SMILES: c1ccc(-c2cccc(N(c3ccc(-n4c5ccccc5c5ccc6ccccc6c54)cc3)c3ccc([Si](c4ccccc4)(c4ccccc4)c4ccccc4)cc3)c2)cc1 ; c1ccc(-c2cccc(N(c3ccc(-n4c5ccccc5c5cc6ccccc6cc54)cc3)c3ccc(C(c4ccccc4)(c4ccccc4)c4ccccc4)cc3)c2)cc1]

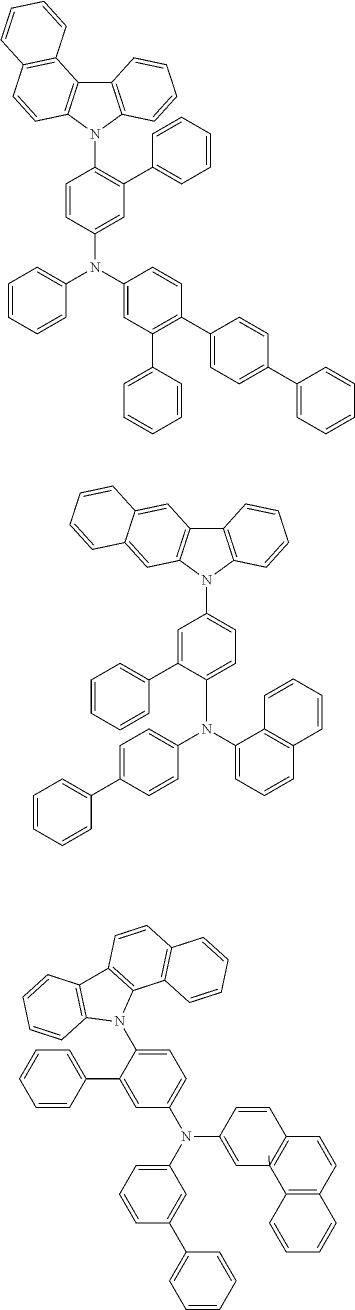

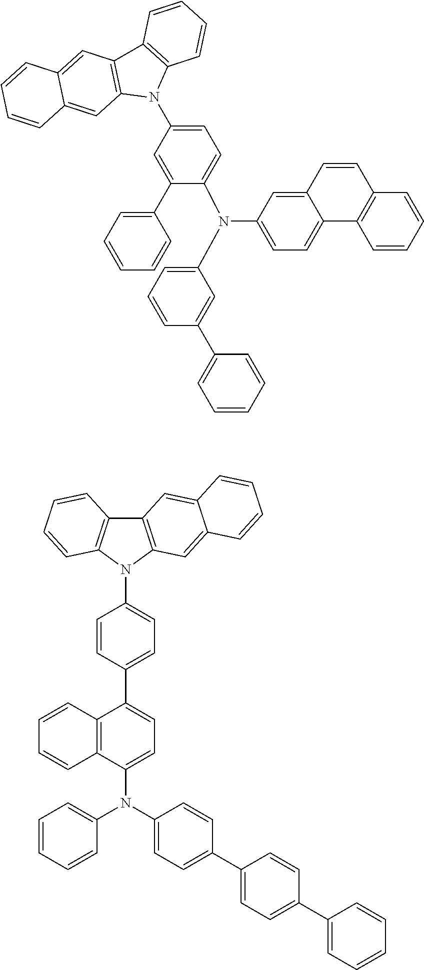 [SMILES: c1ccc(-c2cccc(N(c3ccc4c(ccc5ccccc54)c3)c3ccc(-n4c5ccccc5c5cc6ccccc6cc54)cc3-c3ccccc3)c2)cc1 ; c1ccc(-c2ccc(-c3ccc(N(c4ccccc4)c4ccc(-c5ccc(-n6c7ccccc7c7cc8ccccc8cc76)cc5)c5ccccc45)cc3)cc2)cc1]

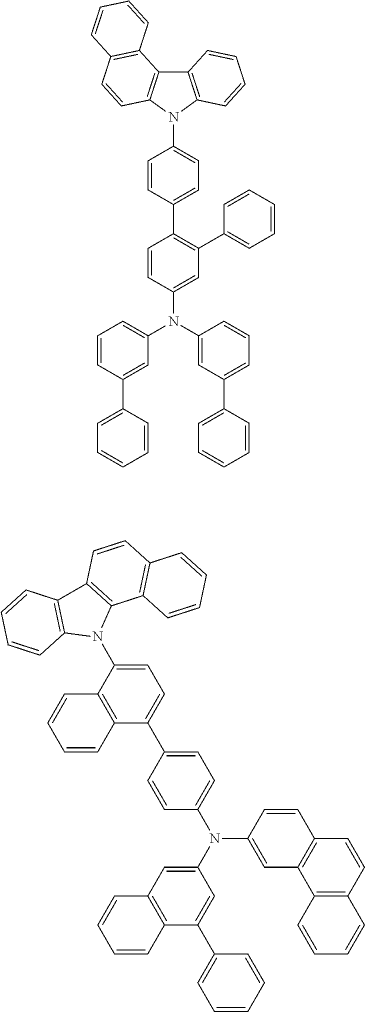 [SMILES: c1ccc(-c2cccc(N(c3cccc(-c4ccccc4)c3)c3ccc(-c4ccc(-n5c6ccccc6c6c7ccccc7ccc65)cc4)c(-c4ccccc4)c3)c2)cc1 ; c1ccc(-c2cc(N(c3ccc(-c4ccc(-n5c6ccccc6c6ccc7ccccc7c65)c5ccccc45)cc3)c3ccc4ccc5ccccc5c4c3)cc3ccccc23)cc1]

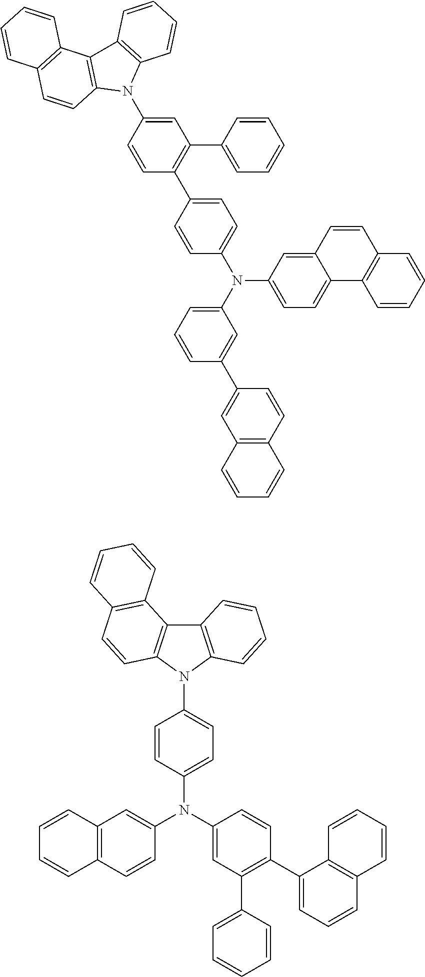 [SMILES: c1ccc(-c2cc(-n3c4ccccc4c4c5ccccc5ccc43)ccc2-c2ccc(N(c3cccc(-c4ccc5ccccc5c4)c3)c3ccc4c(ccc5ccccc54)c3)cc2)cc1 ; c1ccc(-c2cc(N(c3ccc(-n4c5ccccc5c5c6ccccc6ccc54)cc3)c3ccc4ccccc4c3)ccc2-c2cccc3ccccc23)cc1]

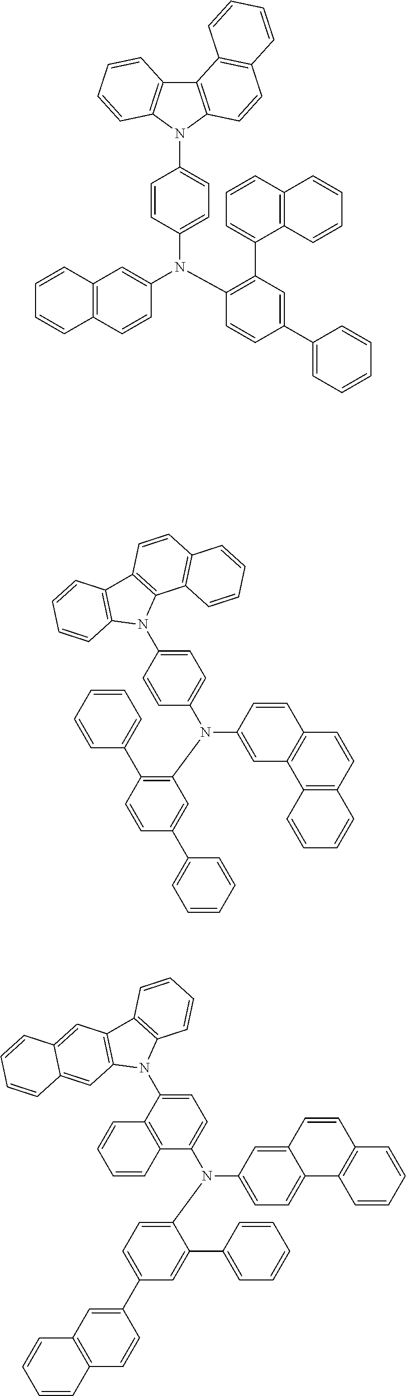 [SMILES: c1ccc(-c2ccc(N(c3ccc(-n4c5ccccc5c5c6ccccc6ccc54)cc3)c3ccc4ccccc4c3)c(-c3cccc4ccccc34)c2)cc1 ; c1ccc(-c2ccc(-c3ccccc3)c(N(c3ccc(-n4c5ccccc5c5ccc6ccccc6c54)cc3)c3ccc4ccc5ccccc5c4c3)c2)cc1 ; c1ccc(-c2cc(-c3ccc4ccccc4c3)ccc2N(c2ccc3c(ccc4ccccc43)c2)c2ccc(-n3c4ccccc4c4cc5ccccc5cc43)c3ccccc23)cc1]

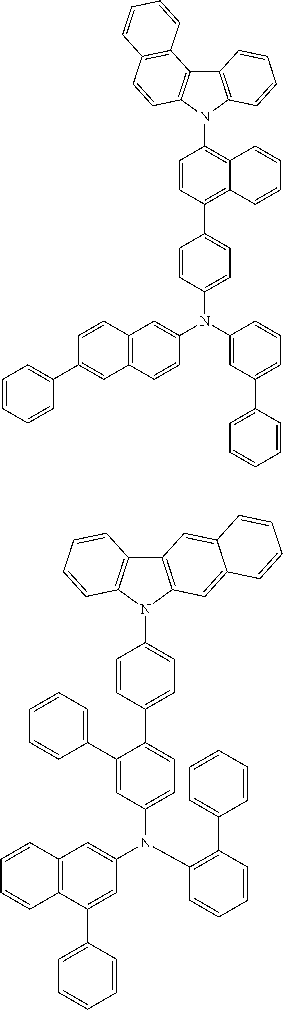 [SMILES: c1ccc(-c2cccc(N(c3ccc(-c4ccc(-n5c6ccccc6c6c7ccccc7ccc65)c5ccccc45)cc3)c3ccc4cc(-c5ccccc5)ccc4c3)c2)cc1 ; c1ccc(-c2cc(N(c3cc(-c4ccccc4)c4ccccc4c3)c3ccccc3-c3ccccc3)ccc2-c2ccc(-n3c4ccccc4c4cc5ccccc5cc43)cc2)cc1]

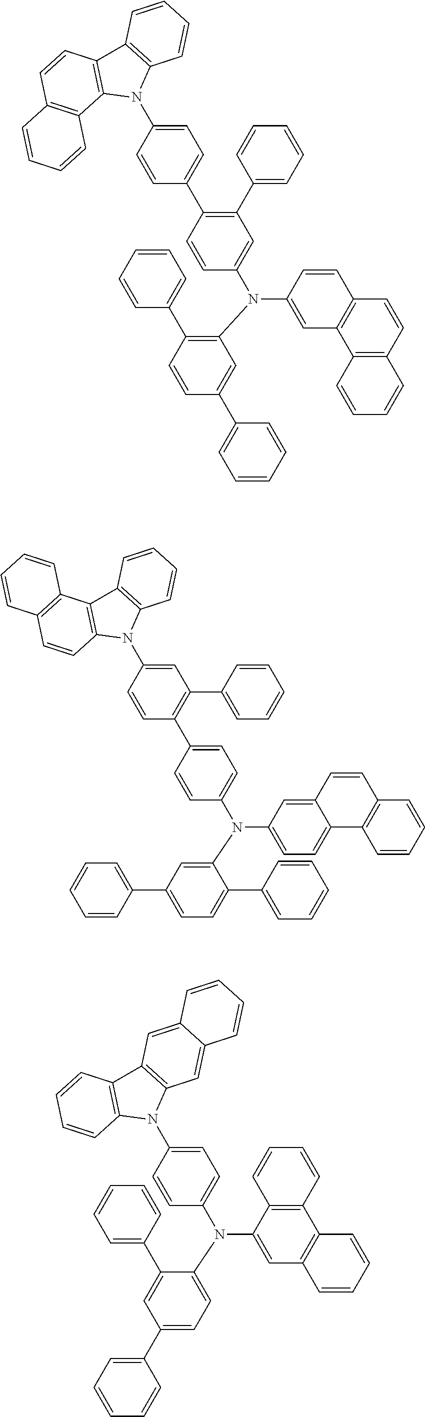 [SMILES: c1ccc(-c2ccc(-c3ccccc3)c(N(c3ccc(-c4ccc(-n5c6ccccc6c6ccc7ccccc7c65)cc4)c(-c4ccccc4)c3)c3ccc4ccc5ccccc5c4c3)c2)cc1 ; c1ccc(-c2ccc(-c3ccccc3)c(N(c3ccc(-c4ccc(-n5c6ccccc6c6c7ccccc7ccc65)cc4-c4ccccc4)cc3)c3ccc4c(ccc5ccccc54)c3)c2)cc1 ; c1ccc(-c2ccc(N(c3ccc(-n4c5ccccc5c5cc6ccccc6cc54)cc3)c3cc4ccccc4c4ccccc34)c(-c3ccccc3)c2)cc1]

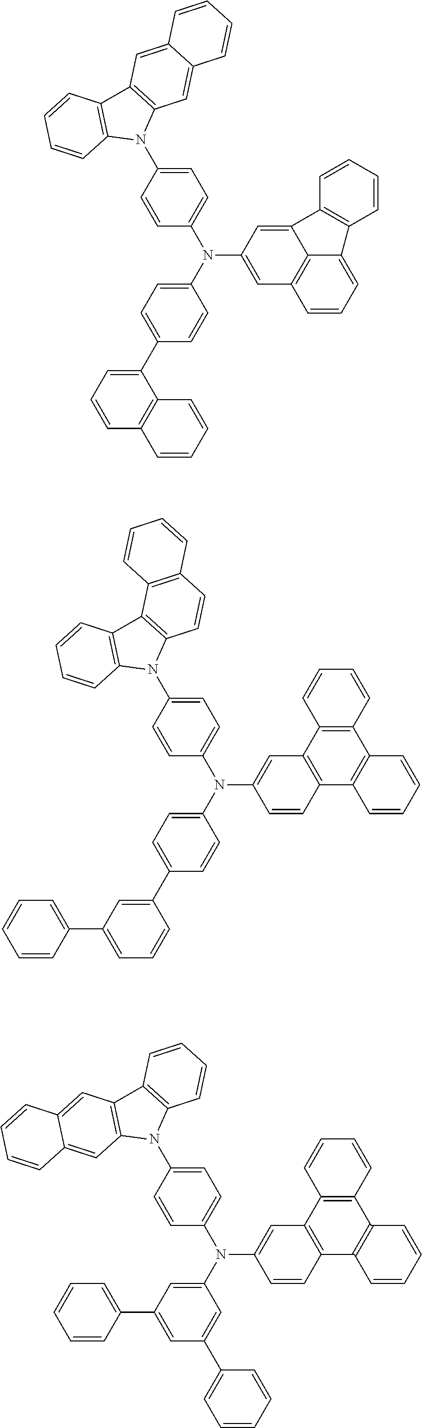 [SMILES: c1ccc2c(c1)-c1cccc3cc(N(c4ccc(-c5cccc6ccccc56)cc4)c4ccc(-n5c6ccccc6c6cc7ccccc7cc65)cc4)cc-2c13 ; c1ccc(-c2cccc(-c3ccc(N(c4ccc(-n5c6ccccc6c6c7ccccc7ccc65)cc4)c4ccc5c6ccccc6c6ccccc6c5c4)cc3)c2)cc1 ; c1ccc(-c2cc(-c3ccccc3)cc(N(c3ccc(-n4c5ccccc5c5cc6ccccc6cc54)cc3)c3ccc4c5ccccc5c5ccccc5c4c3)c2)cc1]

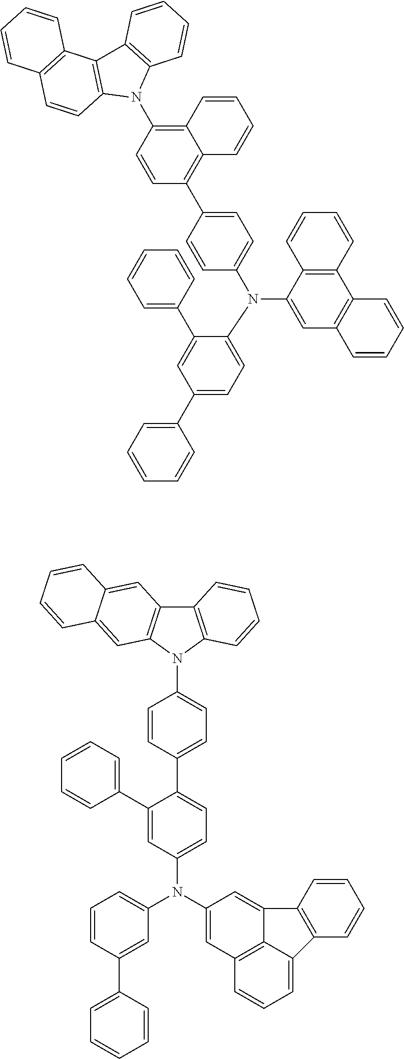 [SMILES: c1ccc(-c2ccc(N(c3ccc(-c4ccc(-n5c6ccccc6c6c7ccccc7ccc65)c5ccccc45)cc3)c3cc4ccccc4c4ccccc34)c(-c3ccccc3)c2)cc1 ; c1ccc(-c2cccc(N(c3ccc(-c4ccc(-n5c6ccccc6c6cc7ccccc7cc65)cc4)c(-c4ccccc4)c3)c3cc4c5c(cccc5c3)-c3ccccc3-4)c2)cc1]

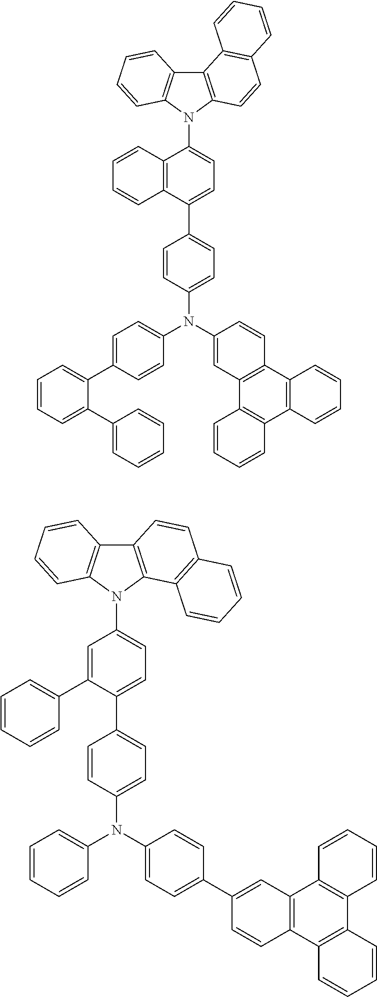 [SMILES: c1ccc(-c2ccccc2-c2ccc(N(c3ccc(-c4ccc(-n5c6ccccc6c6c7ccccc7ccc65)c5ccccc45)cc3)c3ccc4c5ccccc5c5ccccc5c4c3)cc2)cc1 ; c1ccc(-c2cc(-n3c4ccccc4c4ccc5ccccc5c43)ccc2-c2ccc(N(c3ccccc3)c3ccc(-c4ccc5c6ccccc6c6ccccc6c5c4)cc3)cc2)cc1]

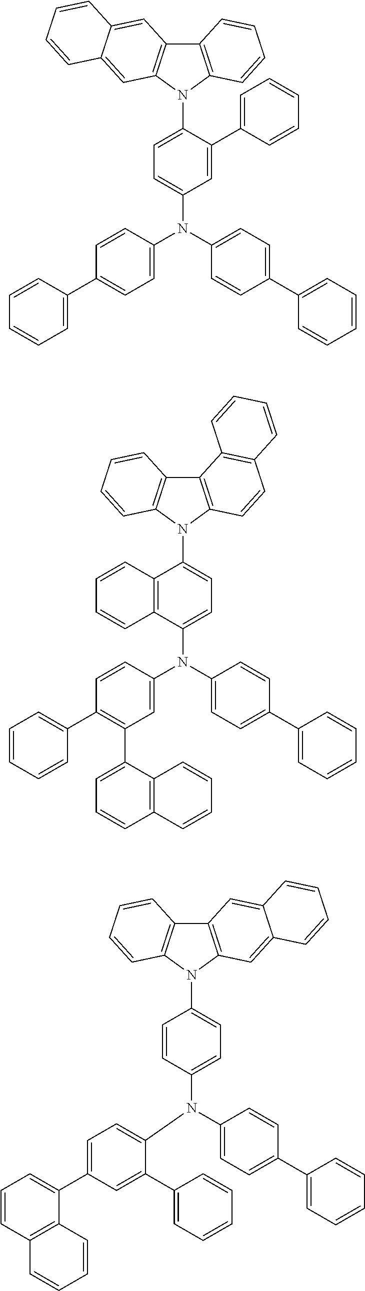 [SMILES: c1ccc(-c2ccc(N(c3ccc(-c4ccccc4)cc3)c3ccc(-n4c5ccccc5c5cc6ccccc6cc54)c(-c4ccccc4)c3)cc2)cc1 ; c1ccc(-c2ccc(N(c3ccc(-c4ccccc4)c(-c4cccc5ccccc45)c3)c3ccc(-n4c5ccccc5c5c6ccccc6ccc54)c4ccccc34)cc2)cc1 ; c1ccc(-c2ccc(N(c3ccc(-n4c5ccccc5c5cc6ccccc6cc54)cc3)c3ccc(-c4cccc5ccccc45)cc3-c3ccccc3)cc2)cc1]

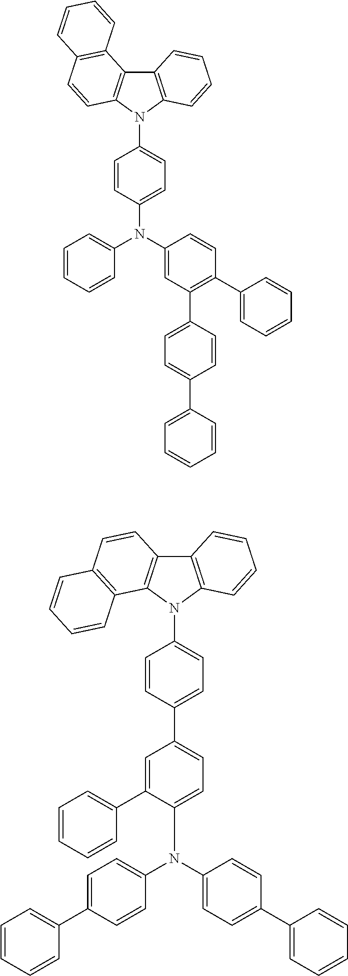 [SMILES: c1ccc(-c2ccc(-c3cc(N(c4ccccc4)c4ccc(-n5c6ccccc6c6c7ccccc7ccc65)cc4)ccc3-c3ccccc3)cc2)cc1 ; c1ccc(-c2ccc(N(c3ccc(-c4ccccc4)cc3)c3ccc(-c4ccc(-n5c6ccccc6c6ccc7ccccc7c65)cc4)cc3-c3ccccc3)cc2)cc1]

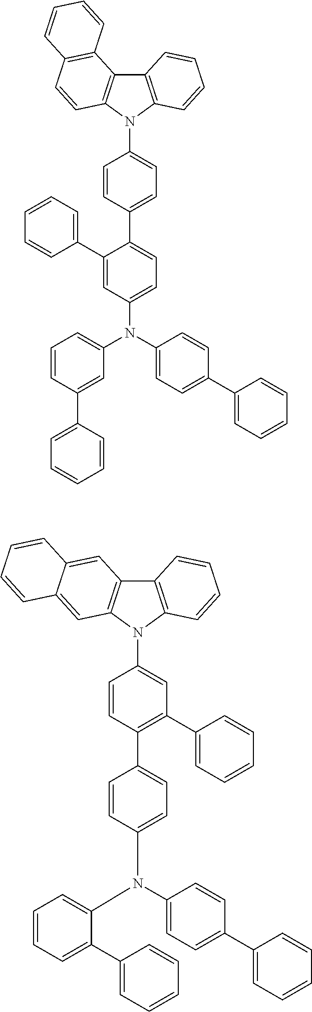 [SMILES: c1ccc(-c2ccc(N(c3cccc(-c4ccccc4)c3)c3ccc(-c4ccc(-n5c6ccccc6c6c7ccccc7ccc65)cc4)c(-c4ccccc4)c3)cc2)cc1 ; c1ccc(-c2ccc(N(c3ccc(-c4ccc(-n5c6ccccc6c6cc7ccccc7cc65)cc4-c4ccccc4)cc3)c3ccccc3-c3ccccc3)cc2)cc1]

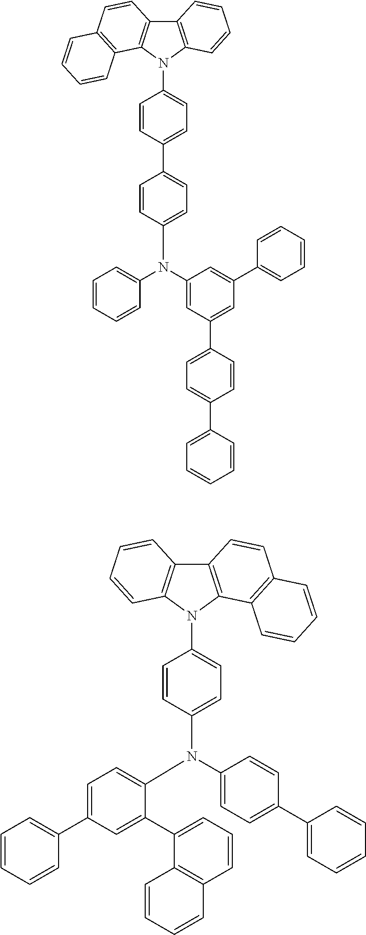 [SMILES: c1ccc(-c2ccc(-c3cc(-c4ccccc4)cc(N(c4ccccc4)c4ccc(-c5ccc(-n6c7ccccc7c7ccc8ccccc8c76)cc5)cc4)c3)cc2)cc1 ; c1ccc(-c2ccc(N(c3ccc(-n4c5ccccc5c5ccc6ccccc6c54)cc3)c3ccc(-c4ccccc4)cc3-c3cccc4ccccc34)cc2)cc1]

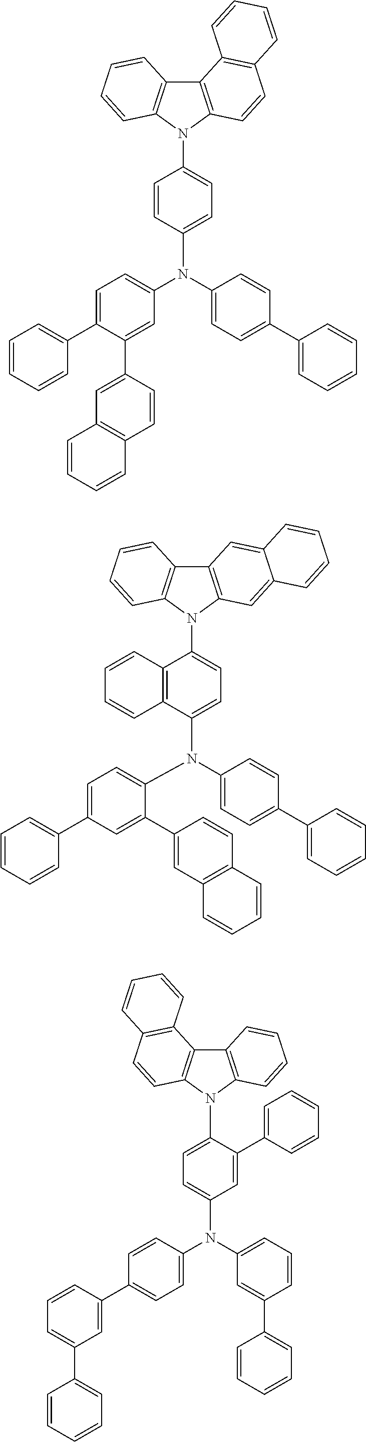 [SMILES: c1ccc(-c2ccc(N(c3ccc(-n4c5ccccc5c5c6ccccc6ccc54)cc3)c3ccc(-c4ccccc4)c(-c4ccc5ccccc5c4)c3)cc2)cc1 ; c1ccc(-c2ccc(N(c3ccc(-c4ccccc4)cc3-c3ccc4ccccc4c3)c3ccc(-n4c5ccccc5c5cc6ccccc6cc54)c4ccccc34)cc2)cc1 ; c1ccc(-c2cccc(-c3ccc(N(c4cccc(-c5ccccc5)c4)c4ccc(-n5c6ccccc6c6c7ccccc7ccc65)c(-c5ccccc5)c4)cc3)c2)cc1]

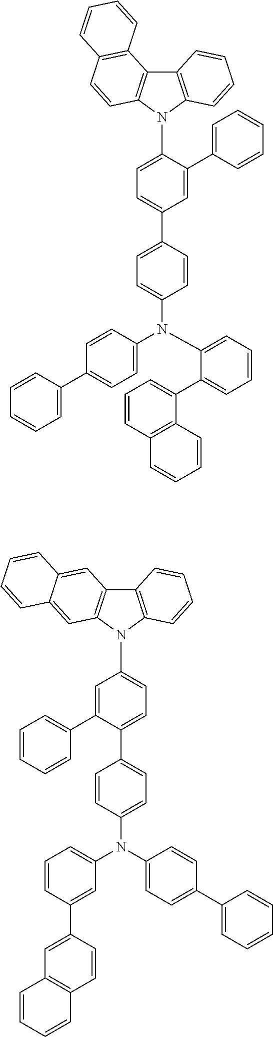 [SMILES: c1ccc(-c2ccc(N(c3ccc(-c4ccc(-n5c6ccccc6c6c7ccccc7ccc65)c(-c5ccccc5)c4)cc3)c3ccccc3-c3cccc4ccccc34)cc2)cc1 ; c1ccc(-c2ccc(N(c3ccc(-c4ccc(-n5c6ccccc6c6cc7ccccc7cc65)cc4-c4ccccc4)cc3)c3cccc(-c4ccc5ccccc5c4)c3)cc2)cc1]

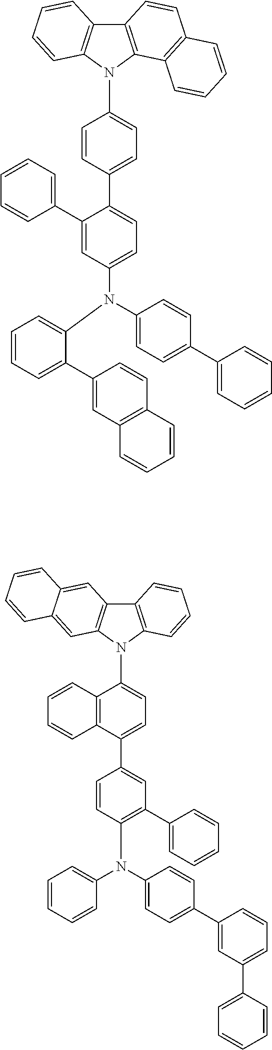 [SMILES: c1ccc(-c2ccc(N(c3ccc(-c4ccc(-n5c6ccccc6c6ccc7ccccc7c65)cc4)c(-c4ccccc4)c3)c3ccccc3-c3ccc4ccccc4c3)cc2)cc1 ; c1ccc(-c2cccc(-c3ccc(N(c4ccccc4)c4ccc(-c5ccc(-n6c7ccccc7c7cc8ccccc8cc76)c6ccccc56)cc4-c4ccccc4)cc3)c2)cc1]

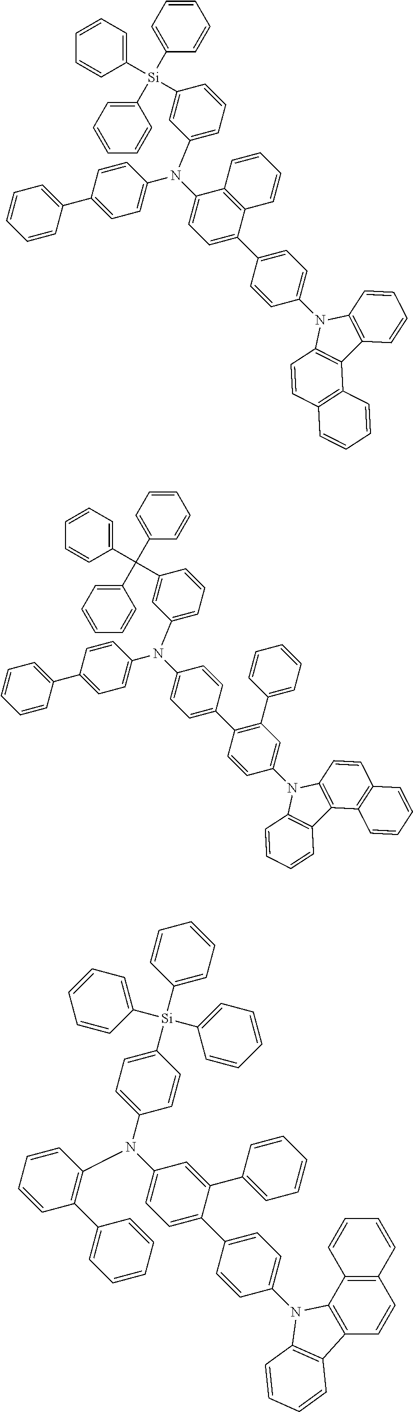 [SMILES: c1ccc(-c2ccc(N(c3cccc([Si](c4ccccc4)(c4ccccc4)c4ccccc4)c3)c3ccc(-c4ccc(-n5c6ccccc6c6c7ccccc7ccc65)cc4)c4ccccc34)cc2)cc1 ; c1ccc(-c2ccc(N(c3ccc(-c4ccc(-n5c6ccccc6c6c7ccccc7ccc65)cc4-c4ccccc4)cc3)c3cccc(C(c4ccccc4)(c4ccccc4)c4ccccc4)c3)cc2)cc1 ; c1ccc(-c2cc(N(c3ccc([Si](c4ccccc4)(c4ccccc4)c4ccccc4)cc3)c3ccccc3-c3ccccc3)ccc2-c2ccc(-n3c4ccccc4c4ccc5ccccc5c43)cc2)cc1]

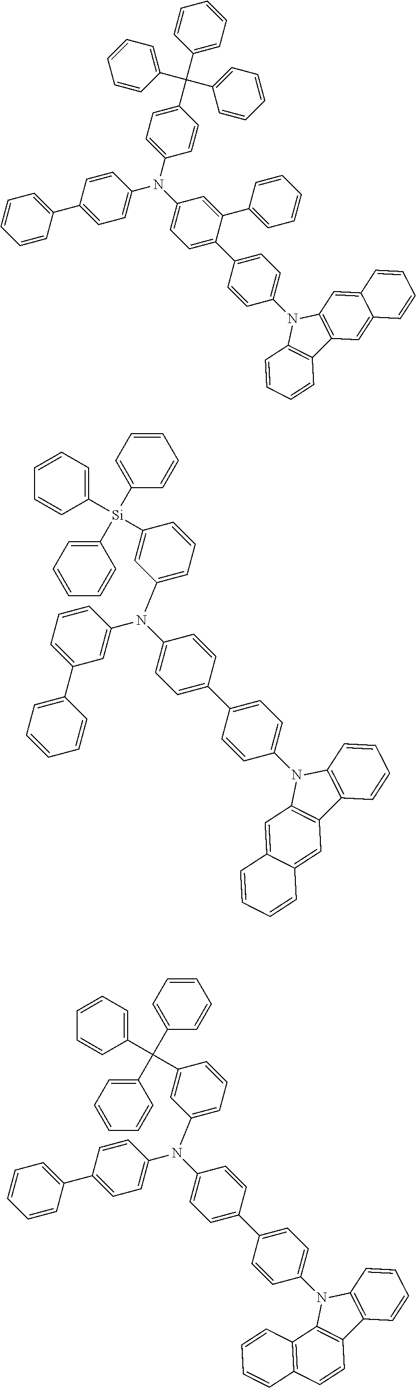 [SMILES: c1ccc(-c2ccc(N(c3ccc(C(c4ccccc4)(c4ccccc4)c4ccccc4)cc3)c3ccc(-c4ccc(-n5c6ccccc6c6cc7ccccc7cc65)cc4)c(-c4ccccc4)c3)cc2)cc1 ; c1ccc(-c2cccc(N(c3ccc(-c4ccc(-n5c6ccccc6c6cc7ccccc7cc65)cc4)cc3)c3cccc([Si](c4ccccc4)(c4ccccc4)c4ccccc4)c3)c2)cc1 ; c1ccc(-c2ccc(N(c3ccc(-c4ccc(-n5c6ccccc6c6ccc7ccccc7c65)cc4)cc3)c3cccc(C(c4ccccc4)(c4ccccc4)c4ccccc4)c3)cc2)cc1]

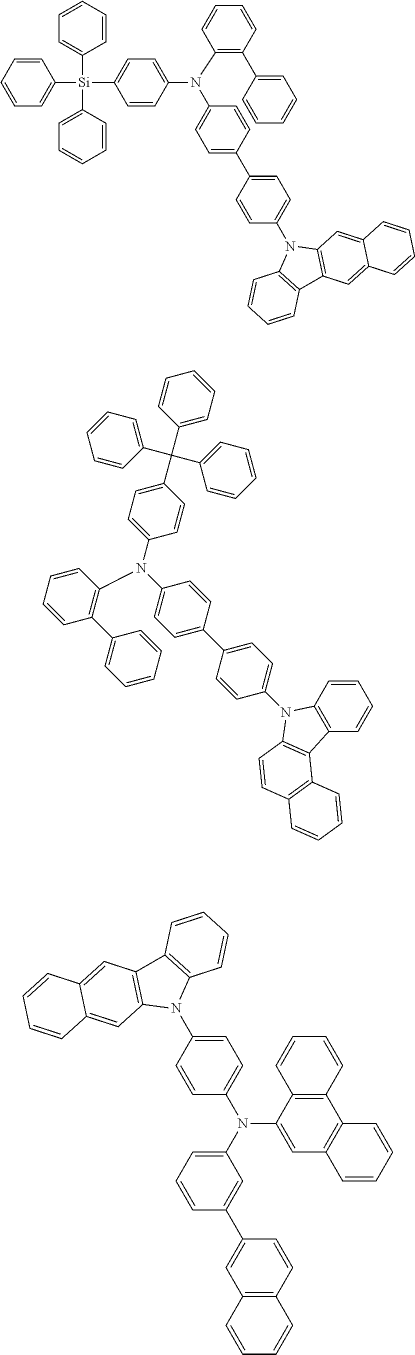 [SMILES: c1ccc(-c2ccccc2N(c2ccc(-c3ccc(-n4c5ccccc5c5cc6ccccc6cc54)cc3)cc2)c2ccc([Si](c3ccccc3)(c3ccccc3)c3ccccc3)cc2)cc1 ; c1ccc(-c2ccccc2N(c2ccc(-c3ccc(-n4c5ccccc5c5c6ccccc6ccc54)cc3)cc2)c2ccc(C(c3ccccc3)(c3ccccc3)c3ccccc3)cc2)cc1 ; c1cc(-c2ccc3ccccc3c2)cc(N(c2ccc(-n3c4ccccc4c4cc5ccccc5cc43)cc2)c2cc3ccccc3c3ccccc23)c1]

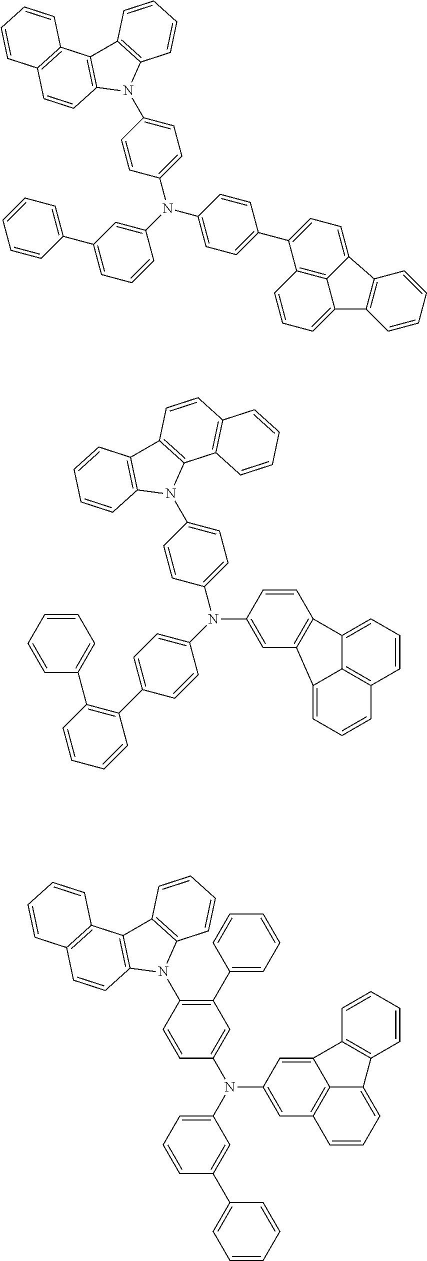 [SMILES: c1ccc(-c2cccc(N(c3ccc(-c4ccc5c6c(cccc46)-c4ccccc4-5)cc3)c3ccc(-n4c5ccccc5c5c6ccccc6ccc54)cc3)c2)cc1 ; c1ccc(-c2ccccc2-c2ccc(N(c3ccc(-n4c5ccccc5c5ccc6ccccc6c54)cc3)c3ccc4c(c3)-c3cccc5cccc-4c35)cc2)cc1 ; c1ccc(-c2cccc(N(c3ccc(-n4c5ccccc5c5c6ccccc6ccc54)c(-c4ccccc4)c3)c3cc4c5c(cccc5c3)-c3ccccc3-4)c2)cc1]

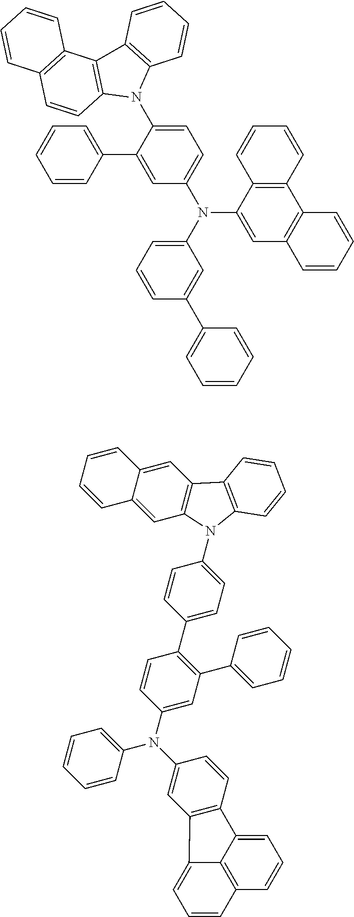 [SMILES: c1ccc(-c2cccc(N(c3ccc(-n4c5ccccc5c5c6ccccc6ccc54)c(-c4ccccc4)c3)c3cc4ccccc4c4ccccc34)c2)cc1 ; c1ccc(-c2cc(N(c3ccccc3)c3ccc4c(c3)-c3cccc5cccc-4c35)ccc2-c2ccc(-n3c4ccccc4c4cc5ccccc5cc43)cc2)cc1]

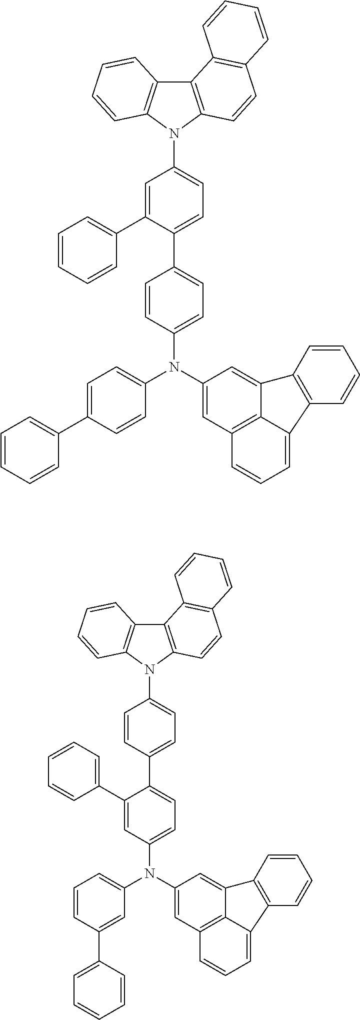 [SMILES: c1ccc(-c2ccc(N(c3ccc(-c4ccc(-n5c6ccccc6c6c7ccccc7ccc65)cc4-c4ccccc4)cc3)c3cc4c5c(cccc5c3)-c3ccccc3-4)cc2)cc1 ; c1ccc(-c2cccc(N(c3ccc(-c4ccc(-n5c6ccccc6c6c7ccccc7ccc65)cc4)c(-c4ccccc4)c3)c3cc4c5c(cccc5c3)-c3ccccc3-4)c2)cc1]

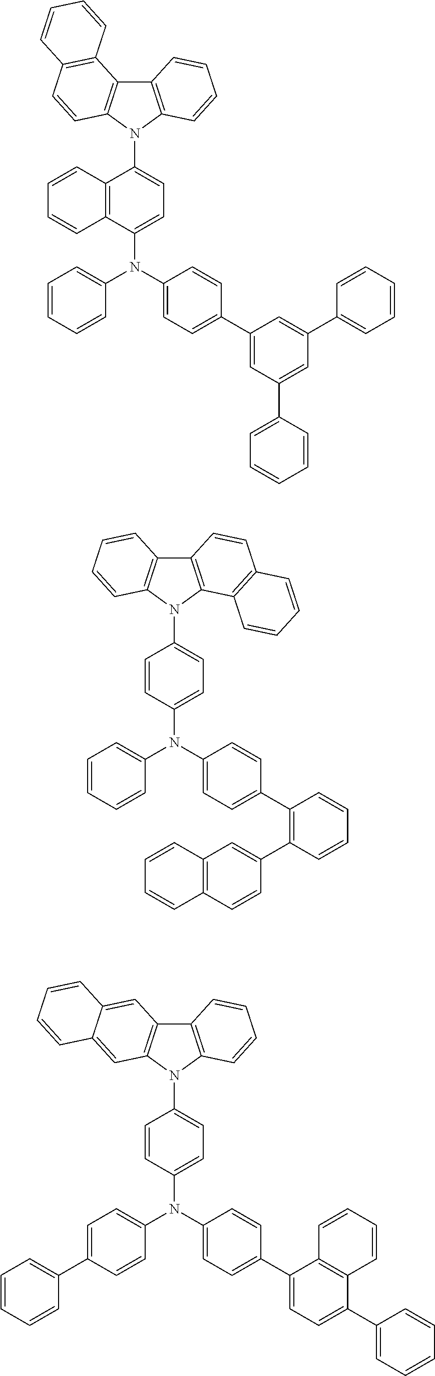 [SMILES: c1ccc(-c2cc(-c3ccccc3)cc(-c3ccc(N(c4ccccc4)c4ccc(-n5c6ccccc6c6c7ccccc7ccc65)c5ccccc45)cc3)c2)cc1 ; c1ccc(N(c2ccc(-c3ccccc3-c3ccc4ccccc4c3)cc2)c2ccc(-n3c4ccccc4c4ccc5ccccc5c43)cc2)cc1 ; c1ccc(-c2ccc(N(c3ccc(-c4ccc(-c5ccccc5)c5ccccc45)cc3)c3ccc(-n4c5ccccc5c5cc6ccccc6cc54)cc3)cc2)cc1]

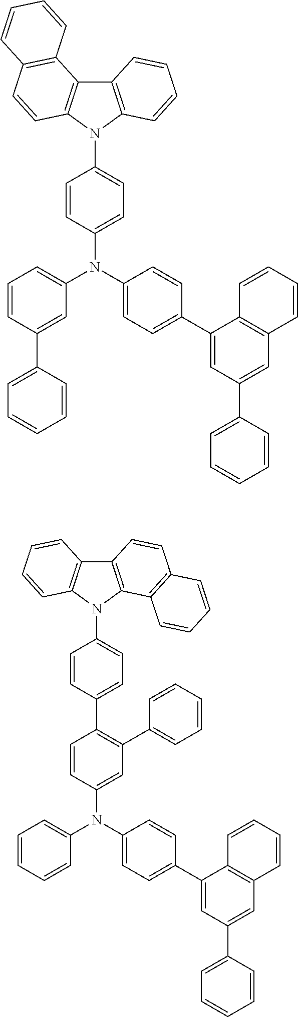 [SMILES: c1ccc(-c2cccc(N(c3ccc(-c4cc(-c5ccccc5)cc5ccccc45)cc3)c3ccc(-n4c5ccccc5c5c6ccccc6ccc54)cc3)c2)cc1 ; c1ccc(-c2cc(-c3ccc(N(c4ccccc4)c4ccc(-c5ccc(-n6c7ccccc7c7ccc8ccccc8c76)cc5)c(-c5ccccc5)c4)cc3)c3ccccc3c2)cc1]

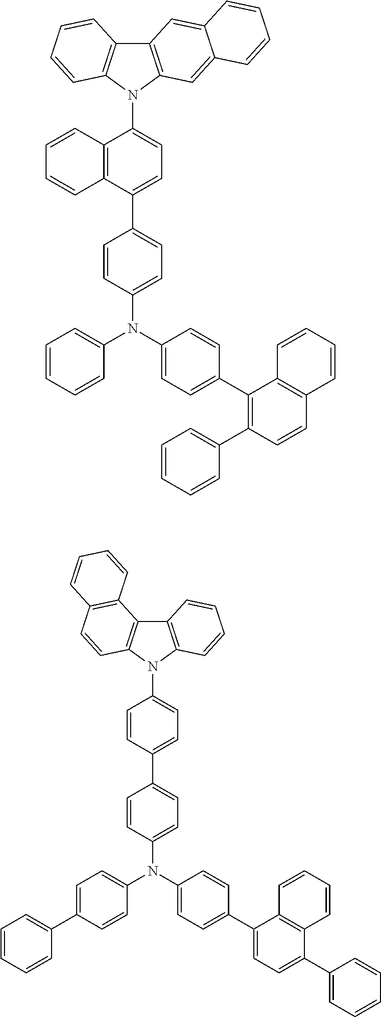 [SMILES: c1ccc(-c2ccc3ccccc3c2-c2ccc(N(c3ccccc3)c3ccc(-c4ccc(-n5c6ccccc6c6cc7ccccc7cc65)c5ccccc45)cc3)cc2)cc1 ; c1ccc(-c2ccc(N(c3ccc(-c4ccc(-n5c6ccccc6c6c7ccccc7ccc65)cc4)cc3)c3ccc(-c4ccc(-c5ccccc5)c5ccccc45)cc3)cc2)cc1]

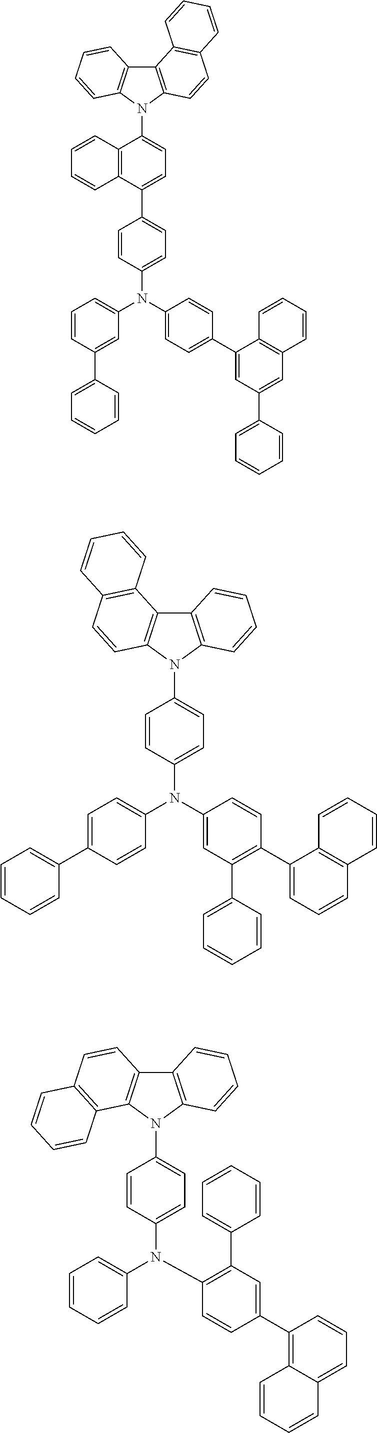 [SMILES: c1ccc(-c2cccc(N(c3ccc(-c4cc(-c5ccccc5)cc5ccccc45)cc3)c3ccc(-c4ccc(-n5c6ccccc6c6c7ccccc7ccc65)c5ccccc45)cc3)c2)cc1 ; c1ccc(-c2ccc(N(c3ccc(-n4c5ccccc5c5c6ccccc6ccc54)cc3)c3ccc(-c4cccc5ccccc45)c(-c4ccccc4)c3)cc2)cc1 ; c1ccc(-c2cc(-c3cccc4ccccc34)ccc2N(c2ccccc2)c2ccc(-n3c4ccccc4c4ccc5ccccc5c43)cc2)cc1]

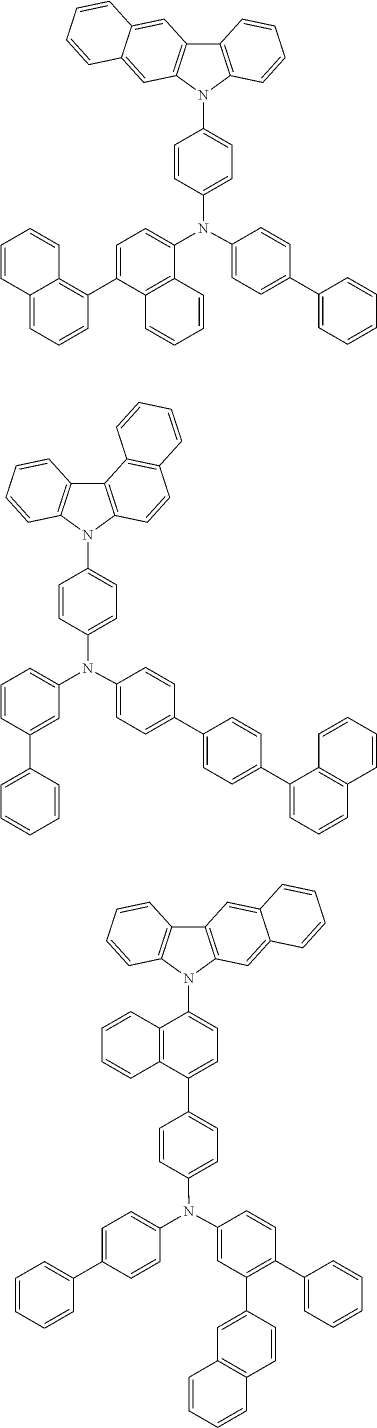 [SMILES: c1ccc(-c2ccc(N(c3ccc(-n4c5ccccc5c5cc6ccccc6cc54)cc3)c3ccc(-c4cccc5ccccc45)c4ccccc34)cc2)cc1 ; c1ccc(-c2cccc(N(c3ccc(-c4ccc(-c5cccc6ccccc56)cc4)cc3)c3ccc(-n4c5ccccc5c5c6ccccc6ccc54)cc3)c2)cc1 ; c1ccc(-c2ccc(N(c3ccc(-c4ccc(-n5c6ccccc6c6cc7ccccc7cc65)c5ccccc45)cc3)c3ccc(-c4ccccc4)c(-c4ccc5ccccc5c4)c3)cc2)cc1]

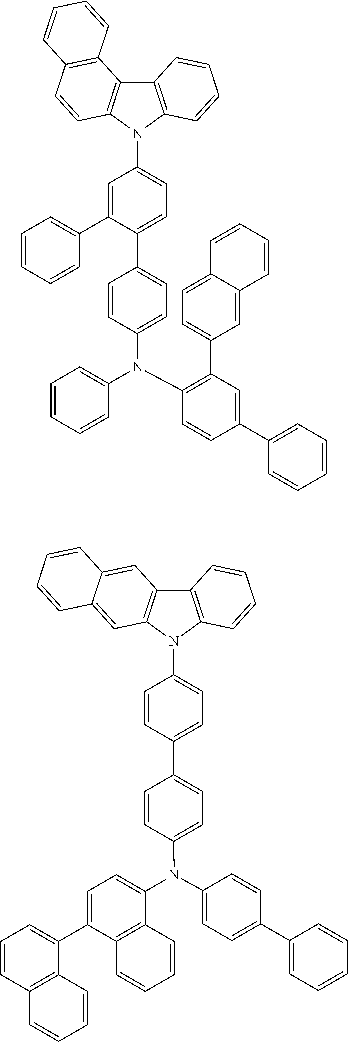 [SMILES: c1ccc(-c2ccc(N(c3ccccc3)c3ccc(-c4ccc(-n5c6ccccc6c6c7ccccc7ccc65)cc4-c4ccccc4)cc3)c(-c3ccc4ccccc4c3)c2)cc1 ; c1ccc(-c2ccc(N(c3ccc(-c4ccc(-n5c6ccccc6c6cc7ccccc7cc65)cc4)cc3)c3ccc(-c4cccc5ccccc45)c4ccccc34)cc2)cc1]

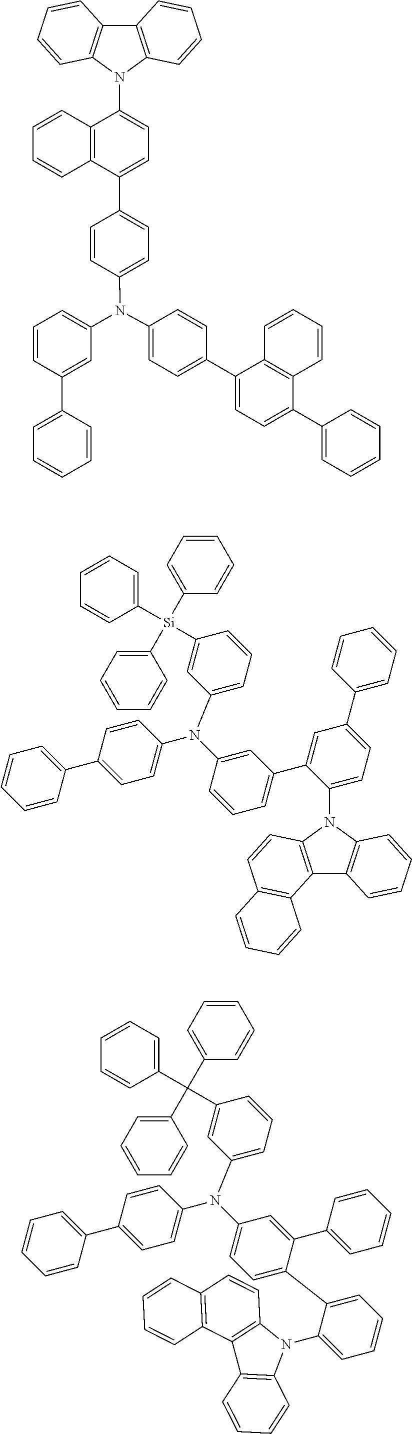 [SMILES: c1ccc(-c2cccc(N(c3ccc(-c4ccc(-c5ccccc5)c5ccccc45)cc3)c3ccc(-c4ccc(-n5c6ccccc6c6ccccc65)c5ccccc45)cc3)c2)cc1 ; c1ccc(-c2ccc(N(c3cccc(-c4cc(-c5ccccc5)ccc4-n4c5ccccc5c5c6ccccc6ccc54)c3)c3cccc([Si](c4ccccc4)(c4ccccc4)c4ccccc4)c3)cc2)cc1 ; c1ccc(-c2ccc(N(c3cccc(C(c4ccccc4)(c4ccccc4)c4ccccc4)c3)c3ccc(-c4ccccc4-n4c5ccccc5c5c6ccccc6ccc54)c(-c4ccccc4)c3)cc2)cc1]

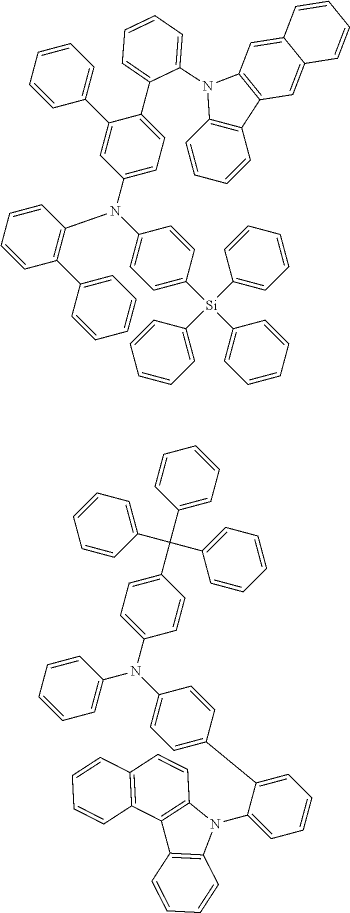 [SMILES: c1ccc(-c2cc(N(c3ccc([Si](c4ccccc4)(c4ccccc4)c4ccccc4)cc3)c3ccccc3-c3ccccc3)ccc2-c2ccccc2-n2c3ccccc3c3cc4ccccc4cc32)cc1 ; c1ccc(N(c2ccc(-c3ccccc3-n3c4ccccc4c4c5ccccc5ccc43)cc2)c2ccc(C(c3ccccc3)(c3ccccc3)c3ccccc3)cc2)cc1]

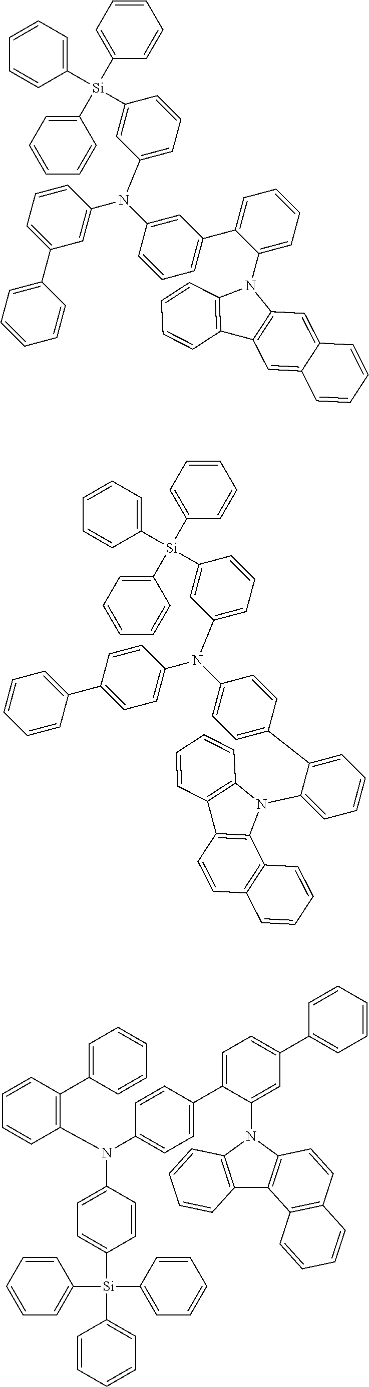 [SMILES: c1ccc(-c2cccc(N(c3cccc(-c4ccccc4-n4c5ccccc5c5cc6ccccc6cc54)c3)c3cccc([Si](c4ccccc4)(c4ccccc4)c4ccccc4)c3)c2)cc1 ; c1ccc(-c2ccc(N(c3ccc(-c4ccccc4-n4c5ccccc5c5ccc6ccccc6c54)cc3)c3cccc([Si](c4ccccc4)(c4ccccc4)c4ccccc4)c3)cc2)cc1 ; c1ccc(-c2ccc(-c3ccc(N(c4ccc([Si](c5ccccc5)(c5ccccc5)c5ccccc5)cc4)c4ccccc4-c4ccccc4)cc3)c(-n3c4ccccc4c4c5ccccc5ccc43)c2)cc1]

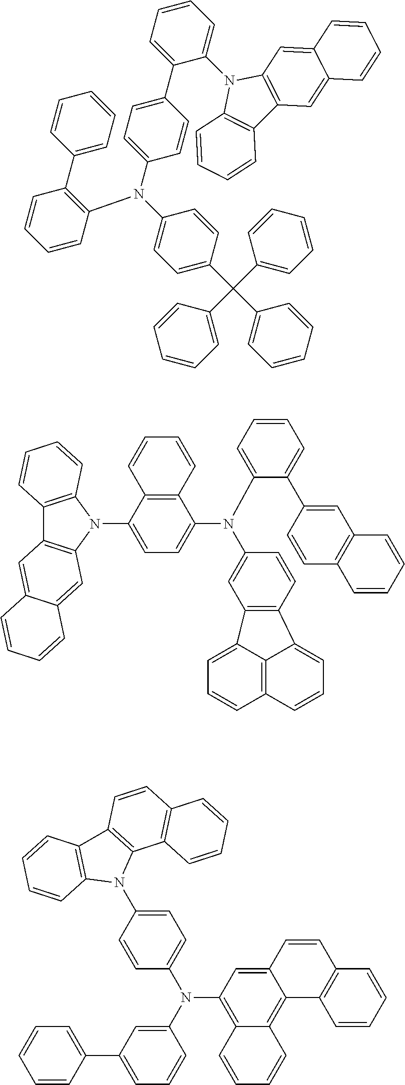 [SMILES: c1ccc(-c2ccccc2N(c2ccc(-c3ccccc3-n3c4ccccc4c4cc5ccccc5cc43)cc2)c2ccc(C(c3ccccc3)(c3ccccc3)c3ccccc3)cc2)cc1 ; c1ccc(N(c2ccc3c(c2)-c2cccc4cccc-3c24)c2ccc(-n3c4ccccc4c4cc5ccccc5cc43)c3ccccc23)c(-c2ccc3ccccc3c2)c1 ; c1ccc(-c2cccc(N(c3ccc(-n4c5ccccc5c5ccc6ccccc6c54)cc3)c3cc4ccc5ccccc5c4c4ccccc34)c2)cc1]

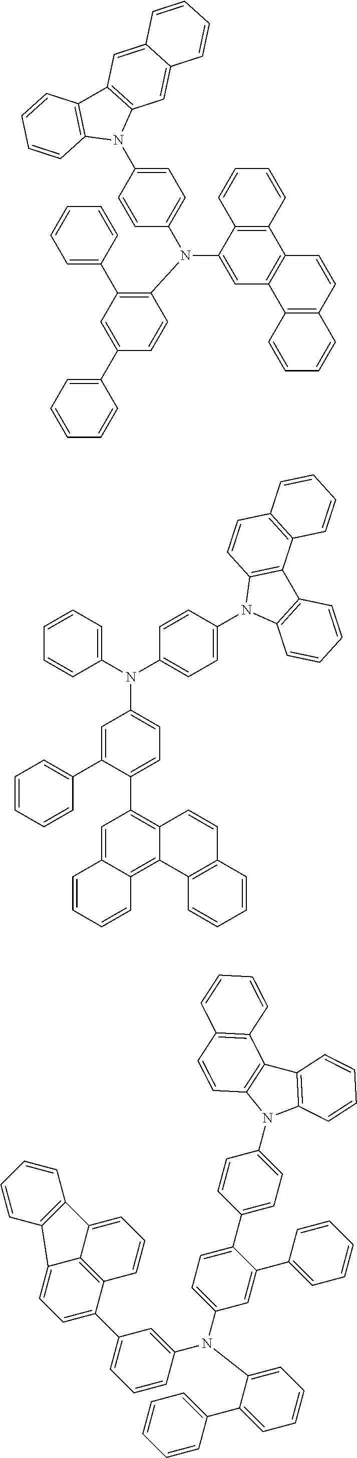 [SMILES: c1ccc(-c2ccc(N(c3ccc(-n4c5ccccc5c5cc6ccccc6cc54)cc3)c3cc4c5ccccc5ccc4c4ccccc34)c(-c3ccccc3)c2)cc1 ; c1ccc(-c2cc(N(c3ccccc3)c3ccc(-n4c5ccccc5c5c6ccccc6ccc54)cc3)ccc2-c2cc3ccccc3c3c2ccc2ccccc23)cc1 ; c1ccc(-c2cc(N(c3cccc(-c4ccc5c6c(cccc46)-c4ccccc4-5)c3)c3ccccc3-c3ccccc3)ccc2-c2ccc(-n3c4ccccc4c4c5ccccc5ccc43)cc2)cc1]

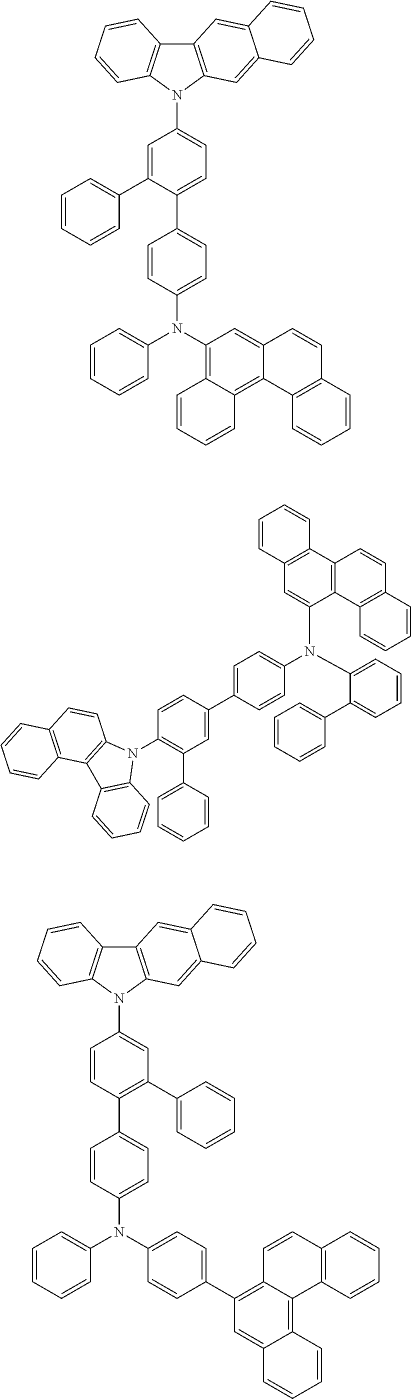 [SMILES: c1ccc(-c2cc(-n3c4ccccc4c4cc5ccccc5cc43)ccc2-c2ccc(N(c3ccccc3)c3cc4ccc5ccccc5c4c4ccccc34)cc2)cc1 ; c1ccc(-c2ccccc2N(c2ccc(-c3ccc(-n4c5ccccc5c5c6ccccc6ccc54)c(-c4ccccc4)c3)cc2)c2cc3ccccc3c3ccc4ccccc4c23)cc1 ; c1ccc(-c2cc(-n3c4ccccc4c4cc5ccccc5cc43)ccc2-c2ccc(N(c3ccccc3)c3ccc(-c4cc5ccccc5c5c4ccc4ccccc45)cc3)cc2)cc1]

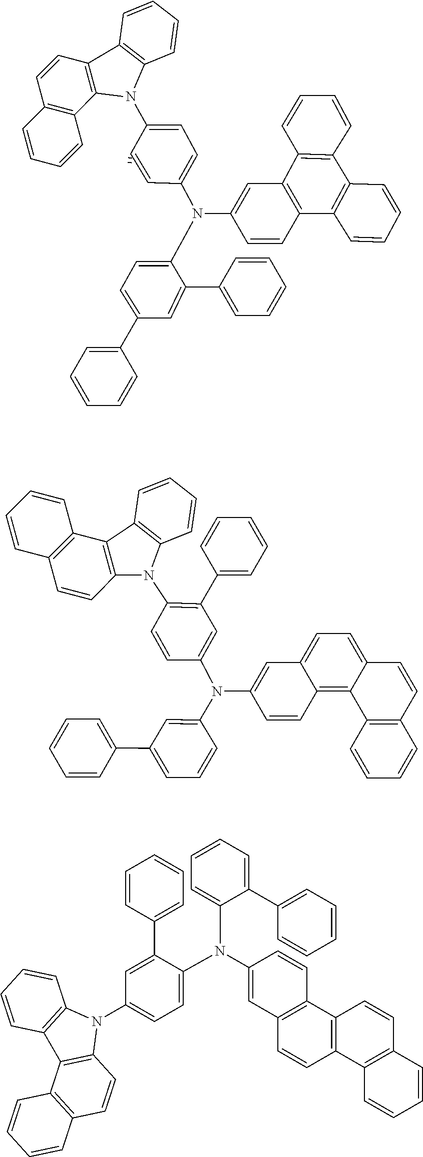

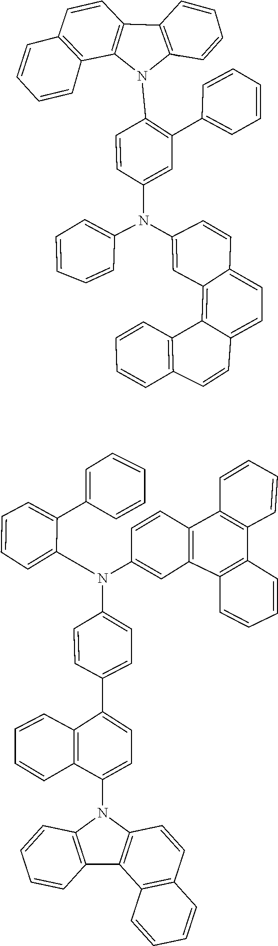

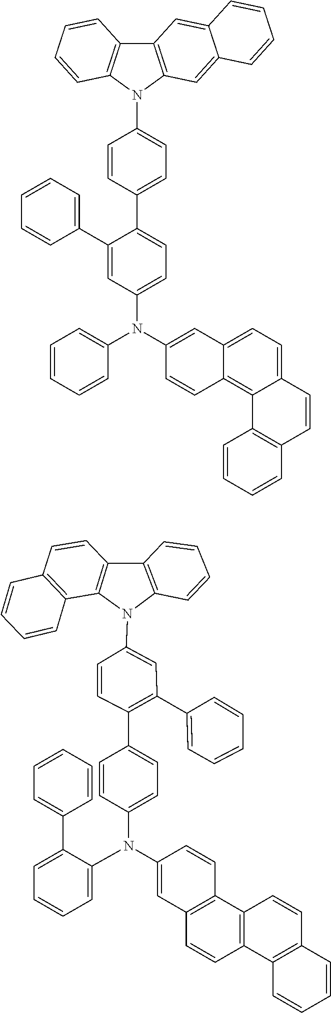 [SMILES: c1ccc(-c2cc(N(c3ccccc3)c3ccc4c(ccc5ccc6ccccc6c54)c3)ccc2-c2ccc(-n3c4ccccc4c4cc5ccccc5cc43)cc2)cc1 ; c1ccc(-c2cc(-n3c4ccccc4c4ccc5ccccc5c43)ccc2-c2ccc(N(c3ccc4c(ccc5c6ccccc6ccc45)c3)c3ccccc3-c3ccccc3)cc2)cc1]

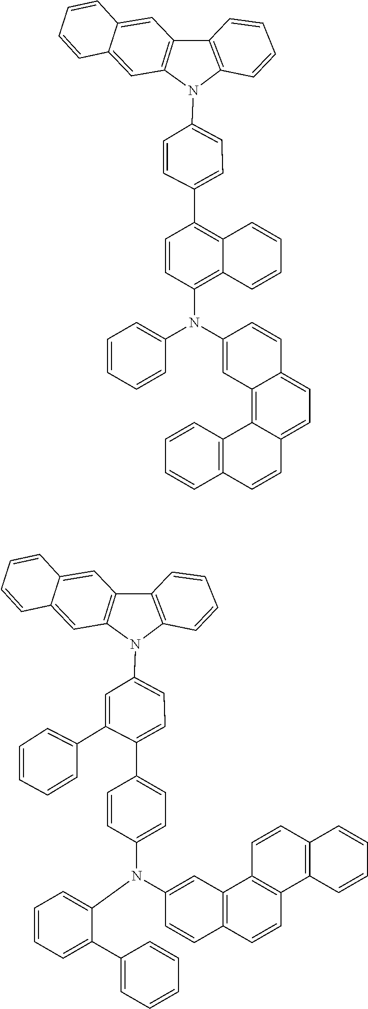 [SMILES: c1ccc(N(c2ccc3ccc4ccc5ccccc5c4c3c2)c2ccc(-c3ccc(-n4c5ccccc5c5cc6ccccc6cc54)cc3)c3ccccc23)cc1 ; c1ccc(-c2cc(-n3c4ccccc4c4cc5ccccc5cc43)ccc2-c2ccc(N(c3ccc4ccc5c6ccccc6ccc5c4c3)c3ccccc3-c3ccccc3)cc2)cc1]

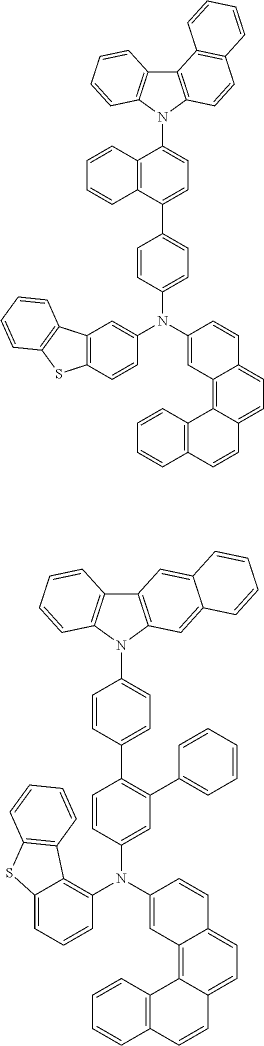 [SMILES: c1ccc2c(c1)ccc1ccc3ccc(N(c4ccc(-c5ccc(-n6c7ccccc7c7c8ccccc8ccc76)c6ccccc56)cc4)c4ccc5sc6ccccc6c5c4)cc3c12 ; c1ccc(-c2cc(N(c3ccc4ccc5ccc6ccccc6c5c4c3)c3cccc4sc5ccccc5c34)ccc2-c2ccc(-n3c4ccccc4c4cc5ccccc5cc43)cc2)cc1]

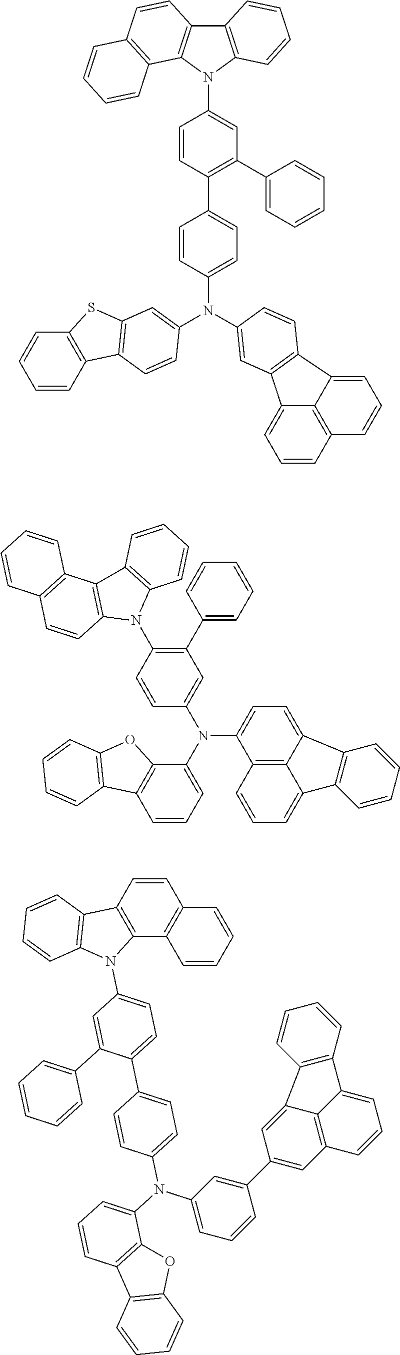 [SMILES: c1ccc(-c2cc(-n3c4ccccc4c4ccc5ccccc5c43)ccc2-c2ccc(N(c3ccc4c(c3)-c3cccc5cccc-4c35)c3ccc4c(c3)sc3ccccc34)cc2)cc1 ; c1ccc(-c2cc(N(c3ccc4c5c(cccc35)-c3ccccc3-4)c3cccc4c3oc3ccccc34)ccc2-n2c3ccccc3c3c4ccccc4ccc32)cc1 ; c1ccc(-c2cc(-n3c4ccccc4c4ccc5ccccc5c43)ccc2-c2ccc(N(c3cccc(-c4cc5c6c(cccc6c4)-c4ccccc4-5)c3)c3cccc4c3oc3ccccc34)cc2)cc1]

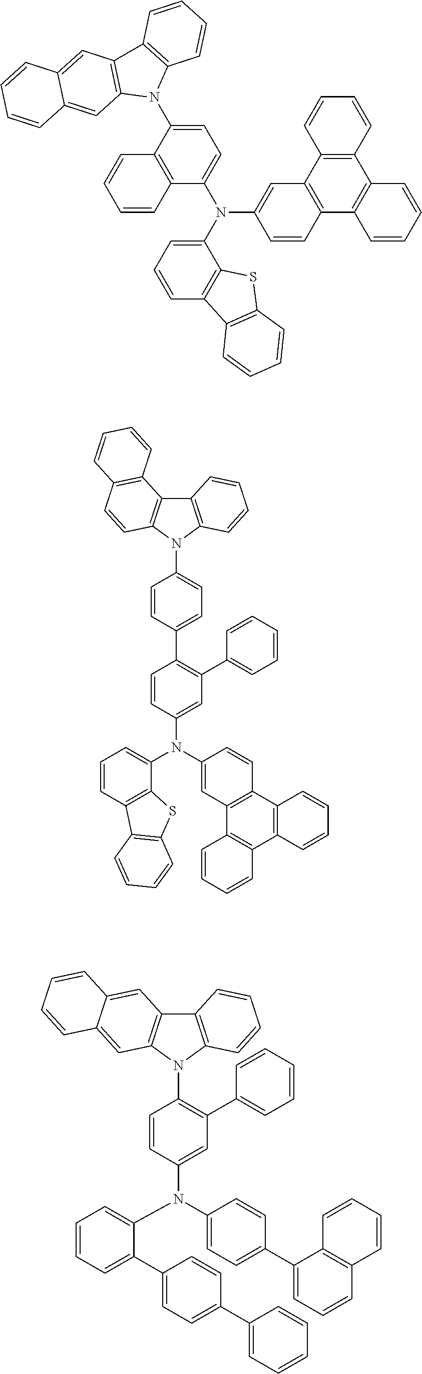 [SMILES: c1ccc2cc3c(cc2c1)c1ccccc1n3-c1ccc(N(c2ccc3c4ccccc4c4ccccc4c3c2)c2cccc3c2sc2ccccc23)c2ccccc12 ; c1ccc(-c2cc(N(c3ccc4c5ccccc5c5ccccc5c4c3)c3cccc4c3sc3ccccc34)ccc2-c2ccc(-n3c4ccccc4c4c5ccccc5ccc43)cc2)cc1 ; c1ccc(-c2ccc(-c3ccccc3N(c3ccc(-c4cccc5ccccc45)cc3)c3ccc(-n4c5ccccc5c5cc6ccccc6cc54)c(-c4ccccc4)c3)cc2)cc1]

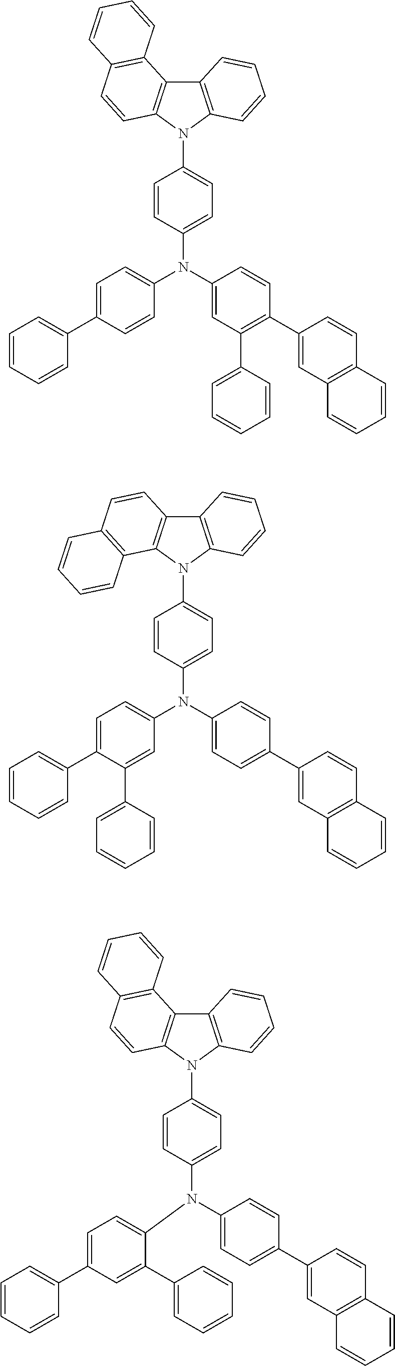 [SMILES: c1ccc(-c2ccc(N(c3ccc(-n4c5ccccc5c5c6ccccc6ccc54)cc3)c3ccc(-c4ccc5ccccc5c4)c(-c4ccccc4)c3)cc2)cc1 ; c1ccc(-c2ccc(N(c3ccc(-c4ccc5ccccc5c4)cc3)c3ccc(-n4c5ccccc5c5ccc6ccccc6c54)cc3)cc2-c2ccccc2)cc1 ; c1ccc(-c2ccc(N(c3ccc(-c4ccc5ccccc5c4)cc3)c3ccc(-n4c5ccccc5c5c6ccccc6ccc54)cc3)c(-c3ccccc3)c2)cc1]

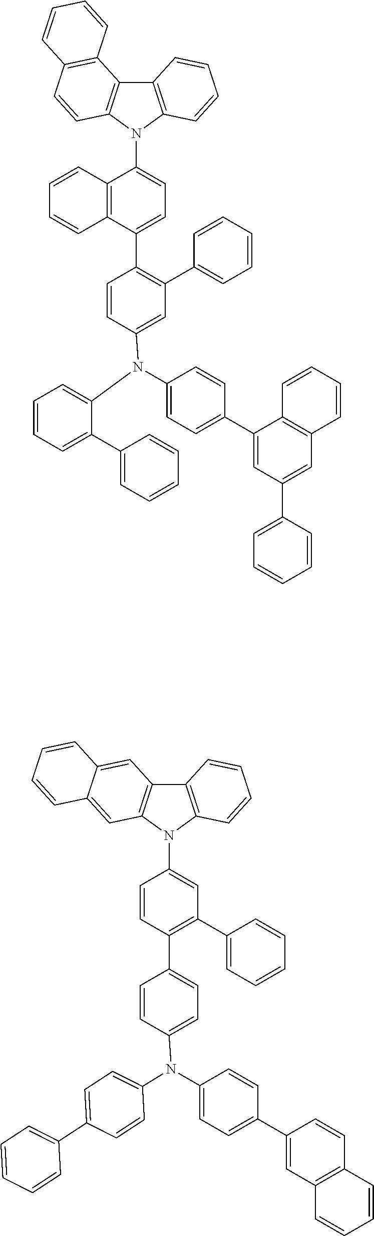 [SMILES: c1ccc(-c2cc(-c3ccc(N(c4ccc(-c5ccc(-n6c7ccccc7c7c8ccccc8ccc76)c6ccccc56)c(-c5ccccc5)c4)c4ccccc4-c4ccccc4)cc3)c3ccccc3c2)cc1 ; c1ccc(-c2ccc(N(c3ccc(-c4ccc5ccccc5c4)cc3)c3ccc(-c4ccc(-n5c6ccccc6c6cc7ccccc7cc65)cc4-c4ccccc4)cc3)cc2)cc1]

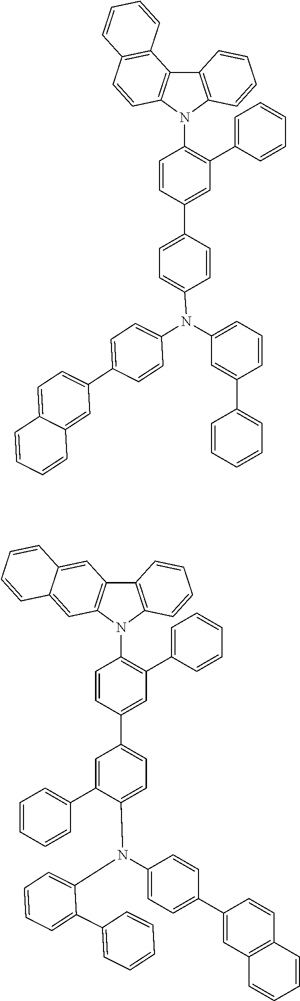 [SMILES: c1ccc(-c2cccc(N(c3ccc(-c4ccc(-n5c6ccccc6c6c7ccccc7ccc65)c(-c5ccccc5)c4)cc3)c3ccc(-c4ccc5ccccc5c4)cc3)c2)cc1 ; c1ccc(-c2ccccc2N(c2ccc(-c3ccc4ccccc4c3)cc2)c2ccc(-c3ccc(-n4c5ccccc5c5cc6ccccc6cc54)c(-c4ccccc4)c3)cc2-c2ccccc2)cc1]

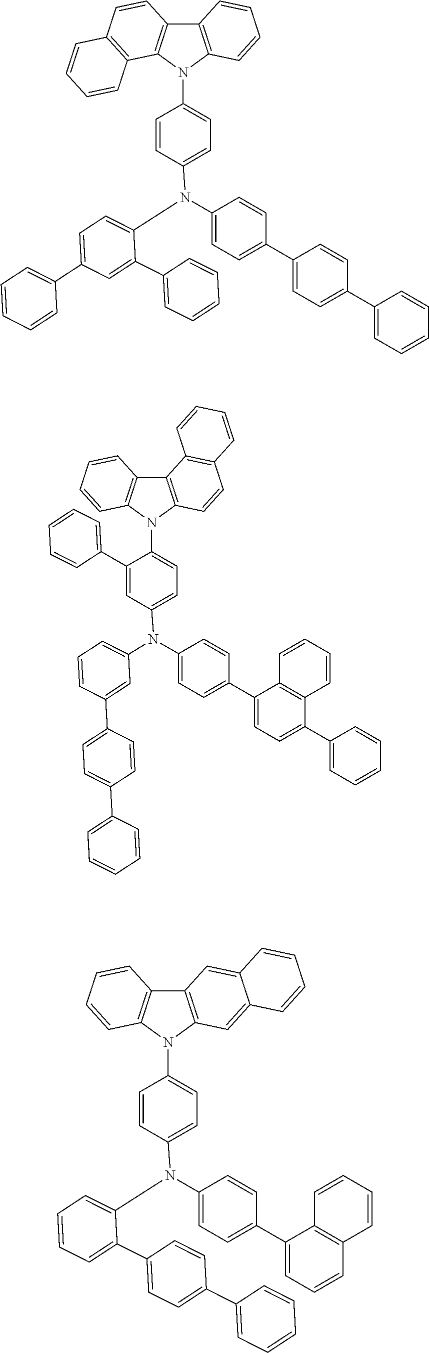 [SMILES: c1ccc(-c2ccc(-c3ccc(N(c4ccc(-n5c6ccccc6c6ccc7ccccc7c65)cc4)c4ccc(-c5ccccc5)cc4-c4ccccc4)cc3)cc2)cc1 ; c1ccc(-c2ccc(-c3cccc(N(c4ccc(-c5ccc(-c6ccccc6)c6ccccc56)cc4)c4ccc(-n5c6ccccc6c6c7ccccc7ccc65)c(-c5ccccc5)c4)c3)cc2)cc1 ; c1ccc(-c2ccc(-c3ccccc3N(c3ccc(-c4cccc5ccccc45)cc3)c3ccc(-n4c5ccccc5c5cc6ccccc6cc54)cc3)cc2)cc1]

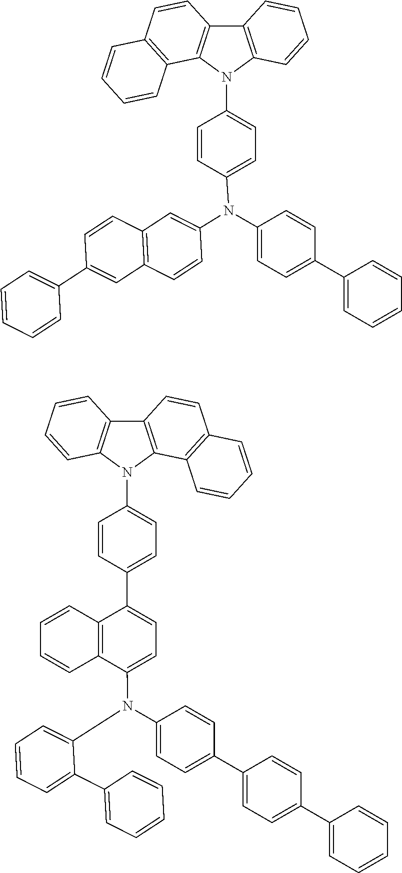 [SMILES: c1ccc(-c2ccc(N(c3ccc(-n4c5ccccc5c5ccc6ccccc6c54)cc3)c3ccc4cc(-c5ccccc5)ccc4c3)cc2)cc1 ; c1ccc(-c2ccc(-c3ccc(N(c4ccccc4-c4ccccc4)c4ccc(-c5ccc(-n6c7ccccc7c7ccc8ccccc8c76)cc5)c5ccccc45)cc3)cc2)cc1]

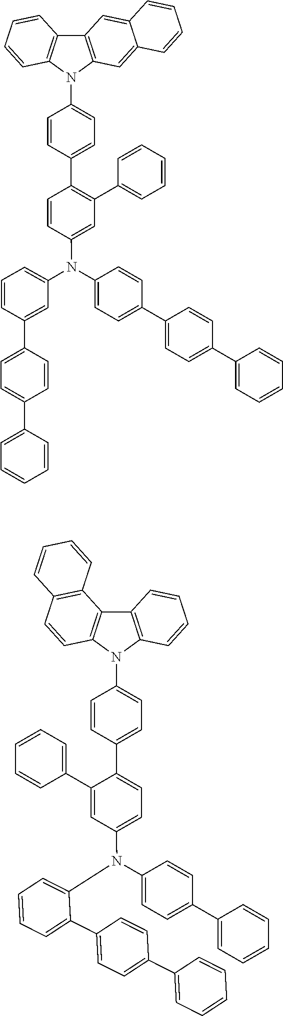 [SMILES: c1ccc(-c2ccc(-c3ccc(N(c4cccc(-c5ccc(-c6ccccc6)cc5)c4)c4ccc(-c5ccc(-n6c7ccccc7c7cc8ccccc8cc76)cc5)c(-c5ccccc5)c4)cc3)cc2)cc1 ; c1ccc(-c2ccc(-c3ccccc3N(c3ccc(-c4ccccc4)cc3)c3ccc(-c4ccc(-n5c6ccccc6c6c7ccccc7ccc65)cc4)c(-c4ccccc4)c3)cc2)cc1]

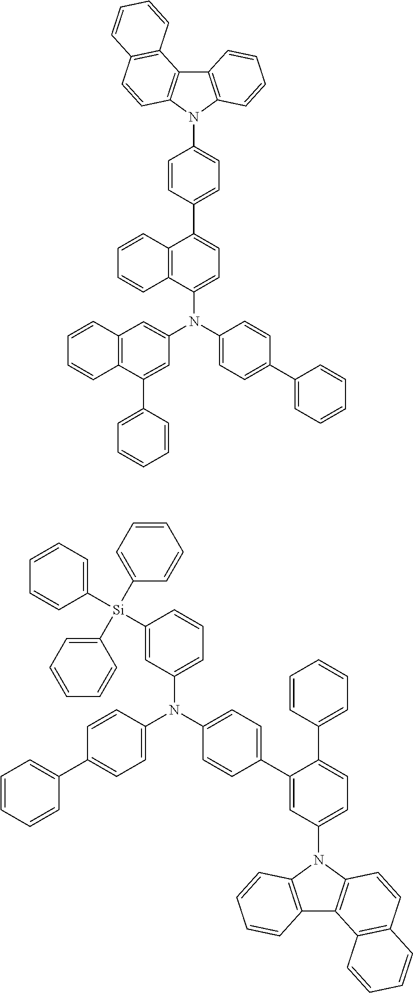 [SMILES: c1ccc(-c2ccc(N(c3cc(-c4ccccc4)c4ccccc4c3)c3ccc(-c4ccc(-n5c6ccccc6c6c7ccccc7ccc65)cc4)c4ccccc34)cc2)cc1 ; c1ccc(-c2ccc(N(c3ccc(-c4cc(-n5c6ccccc6c6c7ccccc7ccc65)ccc4-c4ccccc4)cc3)c3cccc([Si](c4ccccc4)(c4ccccc4)c4ccccc4)c3)cc2)cc1]

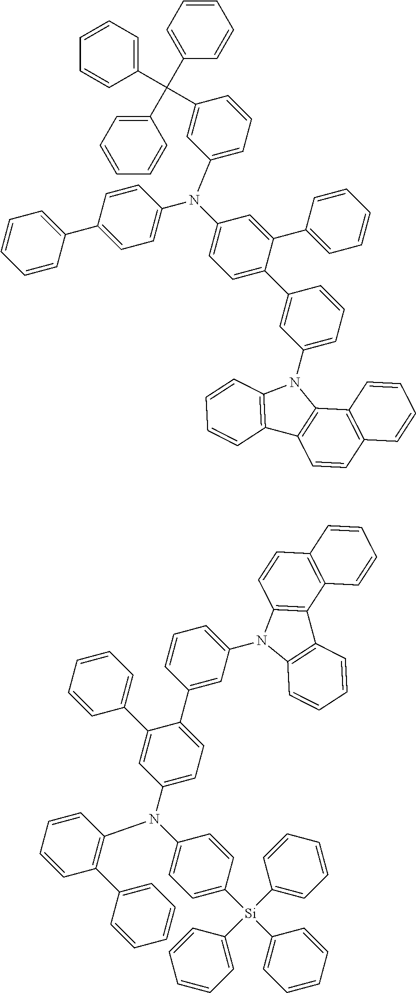 [SMILES: c1ccc(-c2ccc(N(c3cccc(C(c4ccccc4)(c4ccccc4)c4ccccc4)c3)c3ccc(-c4cccc(-n5c6ccccc6c6ccc7ccccc7c65)c4)c(-c4ccccc4)c3)cc2)cc1 ; c1ccc(-c2cc(N(c3ccc([Si](c4ccccc4)(c4ccccc4)c4ccccc4)cc3)c3ccccc3-c3ccccc3)ccc2-c2cccc(-n3c4ccccc4c4c5ccccc5ccc43)c2)cc1]

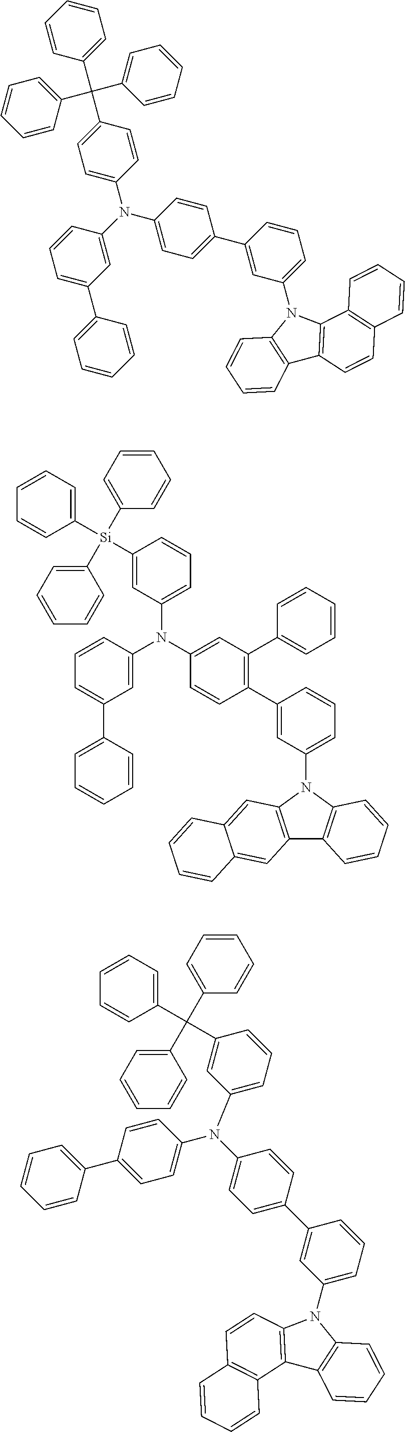 [SMILES: c1ccc(-c2cccc(N(c3ccc(-c4cccc(-n5c6ccccc6c6ccc7ccccc7c65)c4)cc3)c3ccc(C(c4ccccc4)(c4ccccc4)c4ccccc4)cc3)c2)cc1 ; c1ccc(-c2cccc(N(c3cccc([Si](c4ccccc4)(c4ccccc4)c4ccccc4)c3)c3ccc(-c4cccc(-n5c6ccccc6c6cc7ccccc7cc65)c4)c(-c4ccccc4)c3)c2)cc1 ; c1ccc(-c2ccc(N(c3ccc(-c4cccc(-n5c6ccccc6c6c7ccccc7ccc65)c4)cc3)c3cccc(C(c4ccccc4)(c4ccccc4)c4ccccc4)c3)cc2)cc1]

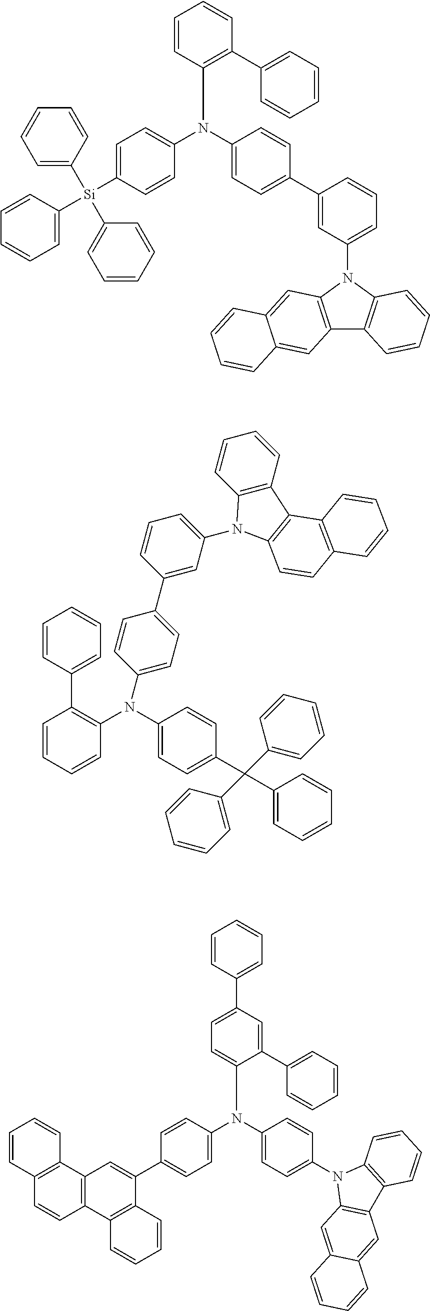 [SMILES: c1ccc(-c2ccccc2N(c2ccc(-c3cccc(-n4c5ccccc5c5cc6ccccc6cc54)c3)cc2)c2ccc([Si](c3ccccc3)(c3ccccc3)c3ccccc3)cc2)cc1 ; c1ccc(-c2ccccc2N(c2ccc(-c3cccc(-n4c5ccccc5c5c6ccccc6ccc54)c3)cc2)c2ccc(C(c3ccccc3)(c3ccccc3)c3ccccc3)cc2)cc1 ; c1ccc(-c2ccc(N(c3ccc(-c4cc5c6ccccc6ccc5c5ccccc45)cc3)c3ccc(-n4c5ccccc5c5cc6ccccc6cc54)cc3)c(-c3ccccc3)c2)cc1]

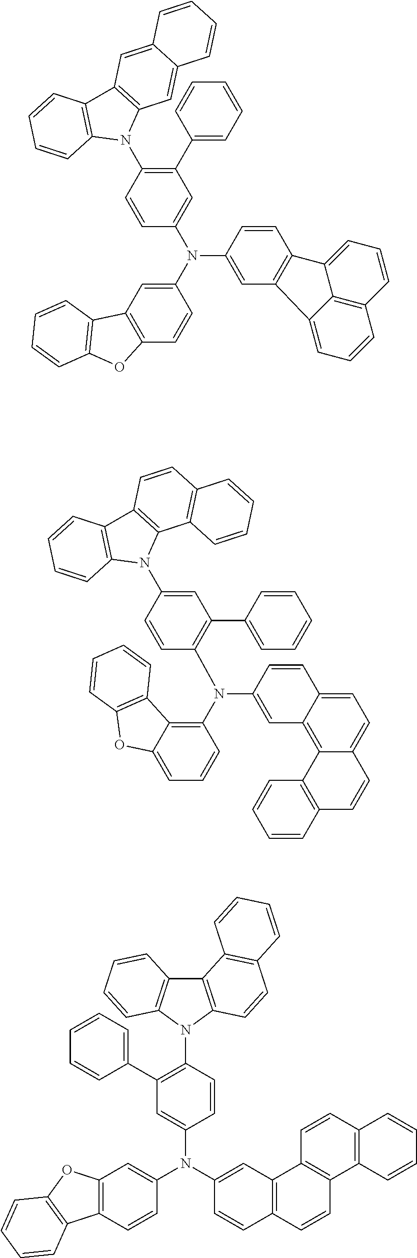 [SMILES: c1ccc(-c2cc(N(c3ccc4c(c3)-c3cccc5cccc-4c35)c3ccc4oc5ccccc5c4c3)ccc2-n2c3ccccc3c3cc4ccccc4cc32)cc1 ; c1ccc(-c2cc(-n3c4ccccc4c4ccc5ccccc5c43)ccc2N(c2ccc3ccc4ccc5ccccc5c4c3c2)c2cccc3oc4ccccc4c23)cc1 ; c1ccc(-c2cc(N(c3ccc4c(c3)oc3ccccc34)c3ccc4ccc5c6ccccc6ccc5c4c3)ccc2-n2c3ccccc3c3c4ccccc4ccc32)cc1]

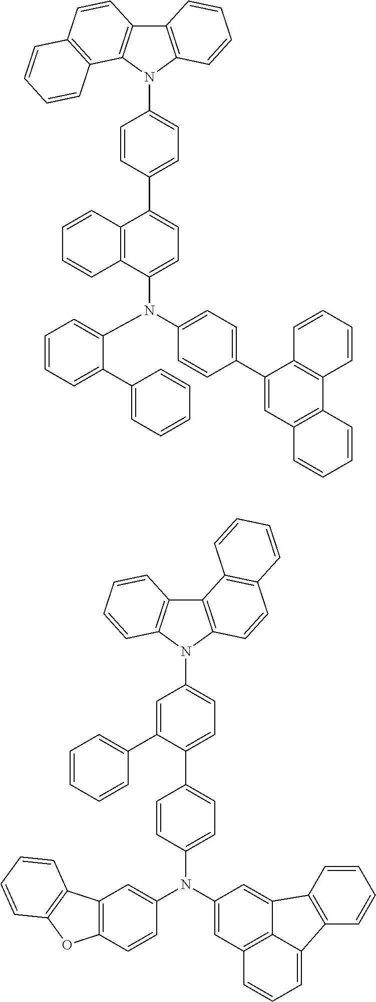 [SMILES: c1ccc(-c2ccccc2N(c2ccc(-c3cc4ccccc4c4ccccc34)cc2)c2ccc(-c3ccc(-n4c5ccccc5c5ccc6ccccc6c54)cc3)c3ccccc23)cc1 ; c1ccc(-c2cc(-n3c4ccccc4c4c5ccccc5ccc43)ccc2-c2ccc(N(c3cc4c5c(cccc5c3)-c3ccccc3-4)c3ccc4oc5ccccc5c4c3)cc2)cc1]

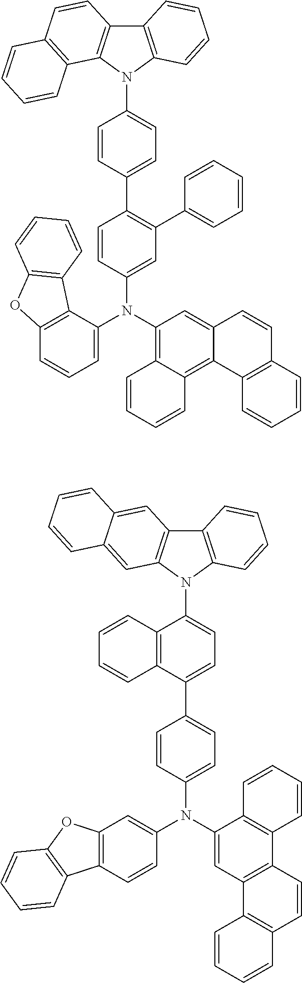 [SMILES: c1ccc(-c2cc(N(c3cc4ccc5ccccc5c4c4ccccc34)c3cccc4oc5ccccc5c34)ccc2-c2ccc(-n3c4ccccc4c4ccc5ccccc5c43)cc2)cc1 ; c1ccc2cc3c(cc2c1)c1ccccc1n3-c1ccc(-c2ccc(N(c3ccc4c(c3)oc3ccccc34)c3cc4c5ccccc5ccc4c4ccccc34)cc2)c2ccccc12]

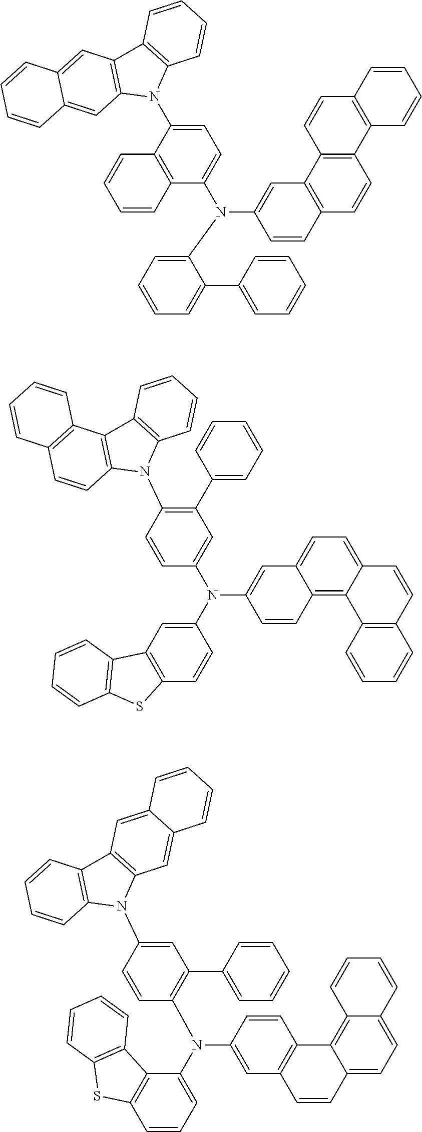 [SMILES: c1ccc(-c2ccccc2N(c2ccc3ccc4c5ccccc5ccc4c3c2)c2ccc(-n3c4ccccc4c4cc5ccccc5cc43)c3ccccc23)cc1 ; c1ccc(-c2cc(N(c3ccc4c(ccc5ccc6ccccc6c54)c3)c3ccc4sc5ccccc5c4c3)ccc2-n2c3ccccc3c3c4ccccc4ccc32)cc1 ; c1ccc(-c2cc(-n3c4ccccc4c4cc5ccccc5cc43)ccc2N(c2ccc3c(ccc4ccc5ccccc5c43)c2)c2cccc3sc4ccccc4c23)cc1]

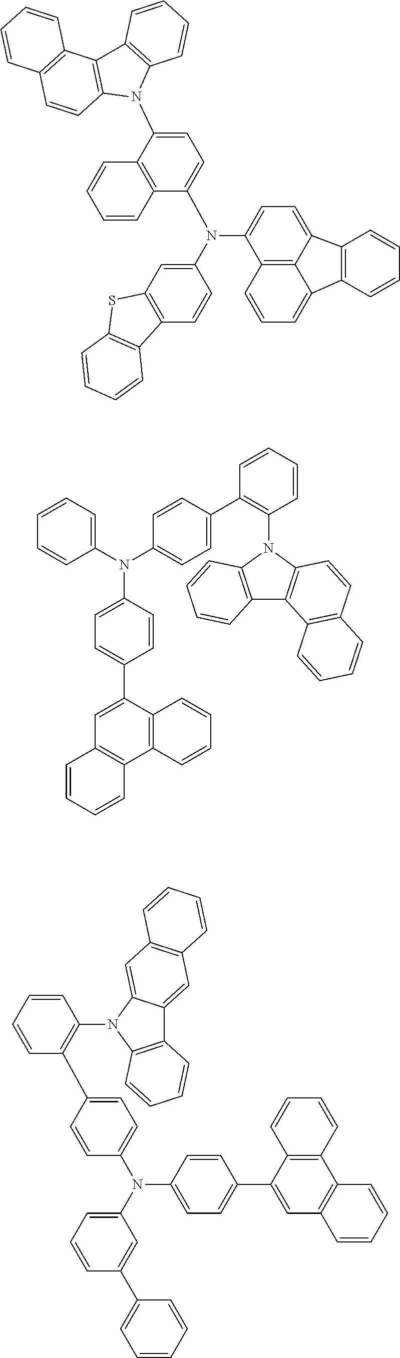 [SMILES: c1ccc2c(c1)-c1cccc3c(N(c4ccc5c(c4)sc4ccccc45)c4ccc(-n5c6ccccc6c6c7ccccc7ccc65)c5ccccc45)ccc-2c13 ; c1ccc(N(c2ccc(-c3ccccc3-n3c4ccccc4c4c5ccccc5ccc43)cc2)c2ccc(-c3cc4ccccc4c4ccccc34)cc2)cc1 ; c1ccc(-c2cccc(N(c3ccc(-c4ccccc4-n4c5ccccc5c5cc6ccccc6cc54)cc3)c3ccc(-c4cc5ccccc5c5ccccc45)cc3)c2)cc1]

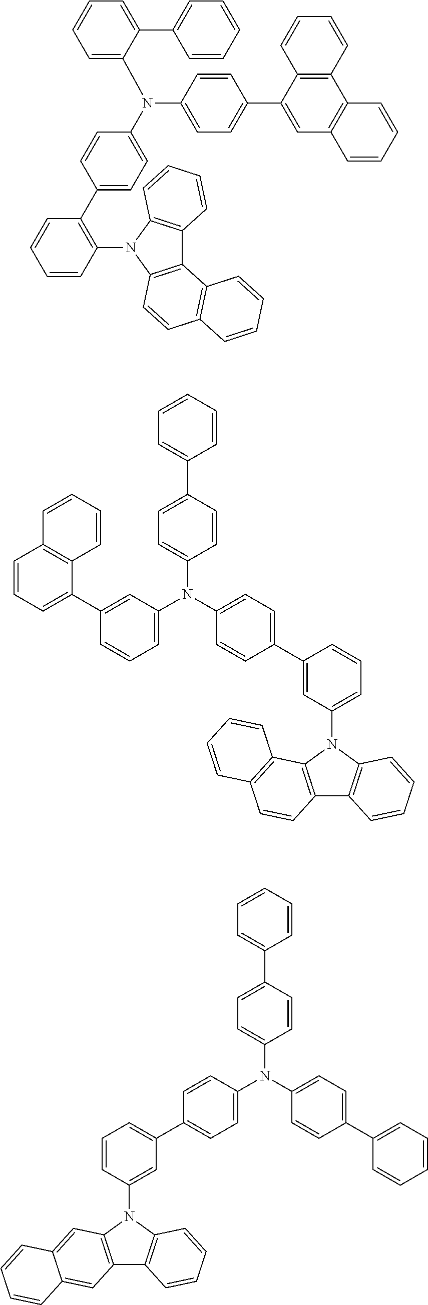 [SMILES: c1ccc(-c2ccccc2N(c2ccc(-c3ccccc3-n3c4ccccc4c4c5ccccc5ccc43)cc2)c2ccc(-c3cc4ccccc4c4ccccc34)cc2)cc1 ; c1ccc(-c2ccc(N(c3ccc(-c4cccc(-n5c6ccccc6c6ccc7ccccc7c65)c4)cc3)c3cccc(-c4cccc5ccccc45)c3)cc2)cc1 ; c1ccc(-c2ccc(N(c3ccc(-c4ccccc4)cc3)c3ccc(-c4cccc(-n5c6ccccc6c6cc7ccccc7cc65)c4)cc3)cc2)cc1]

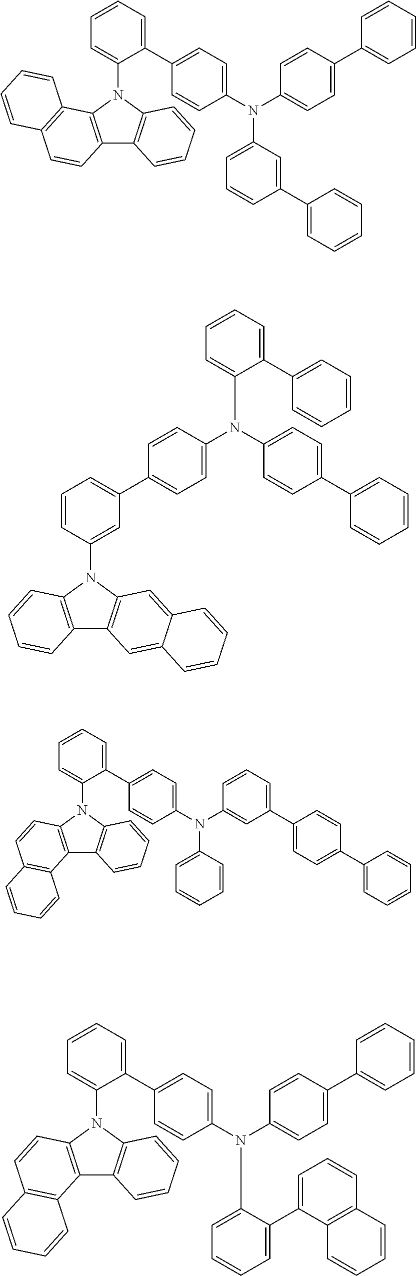 [SMILES: c1ccc(-c2ccc(N(c3ccc(-c4ccccc4-n4c5ccccc5c5ccc6ccccc6c54)cc3)c3cccc(-c4ccccc4)c3)cc2)cc1 ; c1ccc(-c2ccc(N(c3ccc(-c4cccc(-n5c6ccccc6c6cc7ccccc7cc65)c4)cc3)c3ccccc3-c3ccccc3)cc2)cc1 ; c1ccc(-c2ccc(-c3cccc(N(c4ccccc4)c4ccc(-c5ccccc5-n5c6ccccc6c6c7ccccc7ccc65)cc4)c3)cc2)cc1 ; c1ccc(-c2ccc(N(c3ccc(-c4ccccc4-n4c5ccccc5c5c6ccccc6ccc54)cc3)c3ccccc3-c3cccc4ccccc34)cc2)cc1]

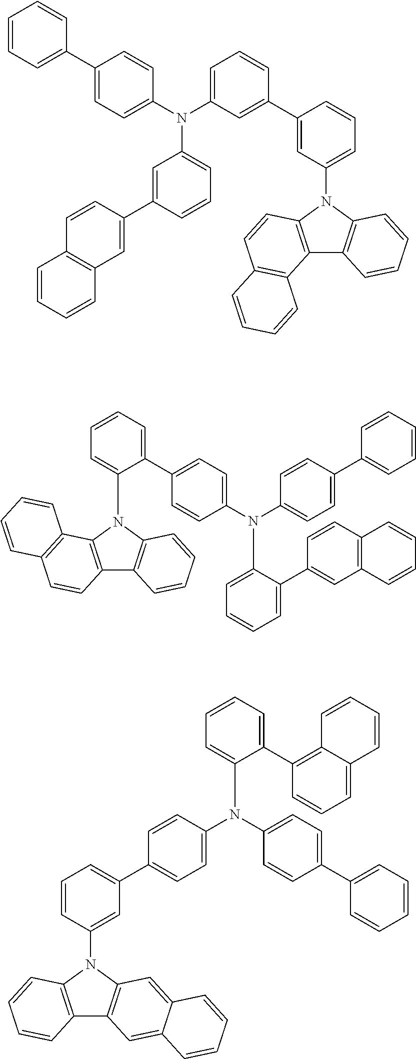 [SMILES: c1ccc(-c2ccc(N(c3cccc(-c4cccc(-n5c6ccccc6c6c7ccccc7ccc65)c4)c3)c3cccc(-c4ccc5ccccc5c4)c3)cc2)cc1 ; c1ccc(-c2ccc(N(c3ccc(-c4ccccc4-n4c5ccccc5c5ccc6ccccc6c54)cc3)c3ccccc3-c3ccc4ccccc4c3)cc2)cc1 ; c1ccc(-c2ccc(N(c3ccc(-c4cccc(-n5c6ccccc6c6cc7ccccc7cc65)c4)cc3)c3ccccc3-c3cccc4ccccc34)cc2)cc1]

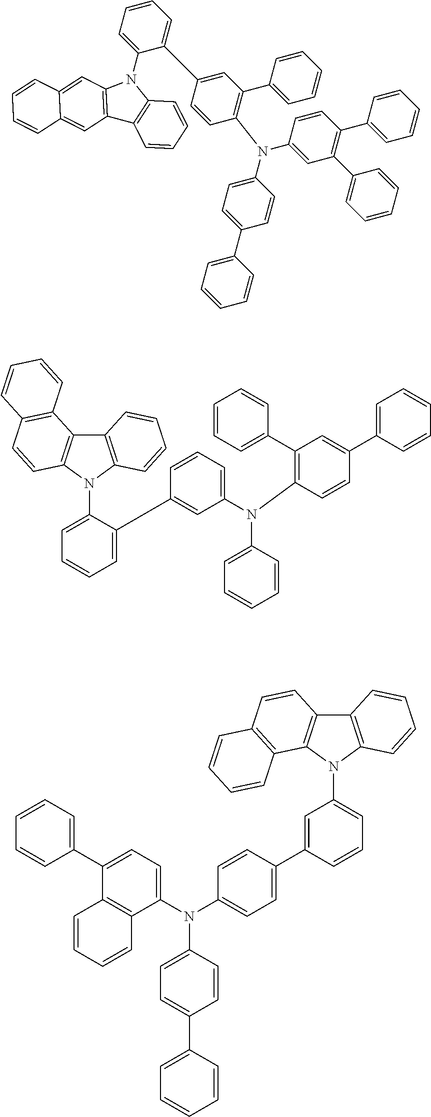 [SMILES: c1ccc(-c2ccc(N(c3ccc(-c4ccccc4)c(-c4ccccc4)c3)c3ccc(-c4ccccc4-n4c5ccccc5c5cc6ccccc6cc54)cc3-c3ccccc3)cc2)cc1 ; c1ccc(-c2ccc(N(c3ccccc3)c3cccc(-c4ccccc4-n4c5ccccc5c5c6ccccc6ccc54)c3)c(-c3ccccc3)c2)cc1 ; c1ccc(-c2ccc(N(c3ccc(-c4cccc(-n5c6ccccc6c6ccc7ccccc7c65)c4)cc3)c3ccc(-c4ccccc4)c4ccccc34)cc2)cc1]

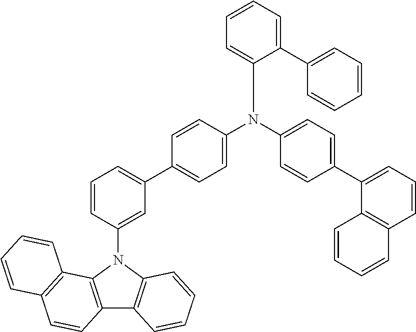 [SMILES: c1ccc(-c2ccccc2N(c2ccc(-c3cccc(-n4c5ccccc5c5ccc6ccccc6c54)c3)cc2)c2ccc(-c3cccc4ccccc34)cc2)cc1]

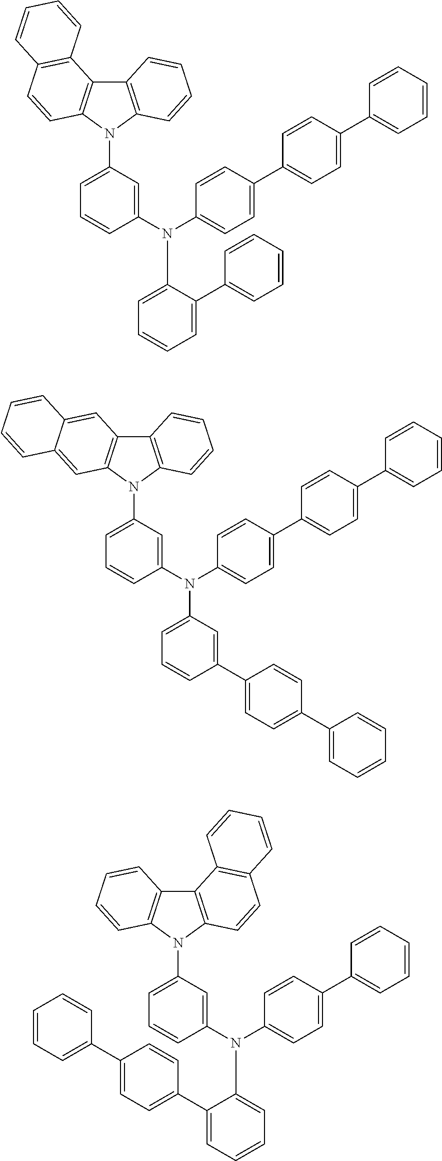 [SMILES: c1ccc(-c2ccc(-c3ccc(N(c4cccc(-n5c6ccccc6c6c7ccccc7ccc65)c4)c4ccccc4-c4ccccc4)cc3)cc2)cc1 ; c1ccc(-c2ccc(-c3ccc(N(c4cccc(-c5ccc(-c6ccccc6)cc5)c4)c4cccc(-n5c6ccccc6c6cc7ccccc7cc65)c4)cc3)cc2)cc1 ; c1ccc(-c2ccc(-c3ccccc3N(c3ccc(-c4ccccc4)cc3)c3cccc(-n4c5ccccc5c5c6ccccc6ccc54)c3)cc2)cc1]

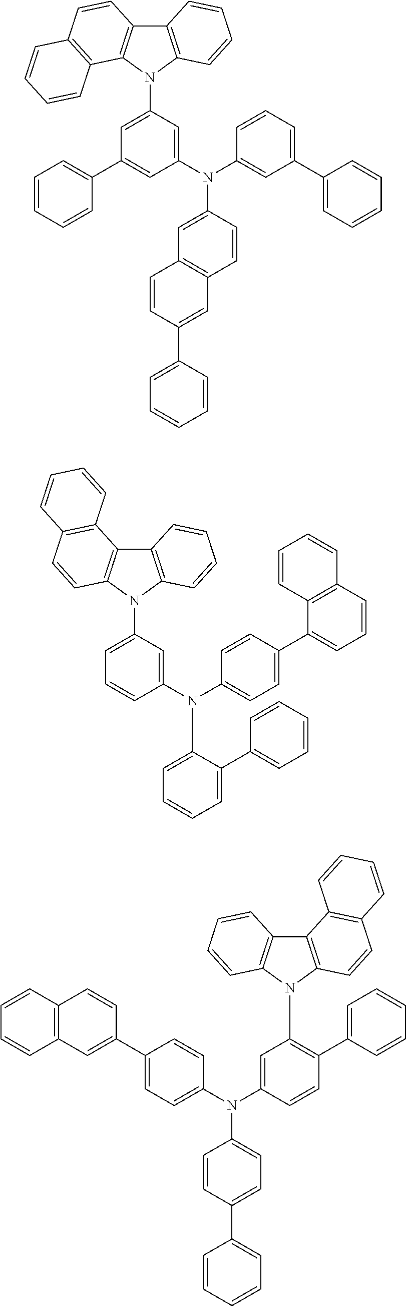 [SMILES: c1ccc(-c2cccc(N(c3cc(-c4ccccc4)cc(-n4c5ccccc5c5ccc6ccccc6c54)c3)c3ccc4cc(-c5ccccc5)ccc4c3)c2)cc1 ; c1ccc(-c2ccccc2N(c2ccc(-c3cccc4ccccc34)cc2)c2cccc(-n3c4ccccc4c4c5ccccc5ccc43)c2)cc1 ; c1ccc(-c2ccc(N(c3ccc(-c4ccc5ccccc5c4)cc3)c3ccc(-c4ccccc4)c(-n4c5ccccc5c5c6ccccc6ccc54)c3)cc2)cc1]

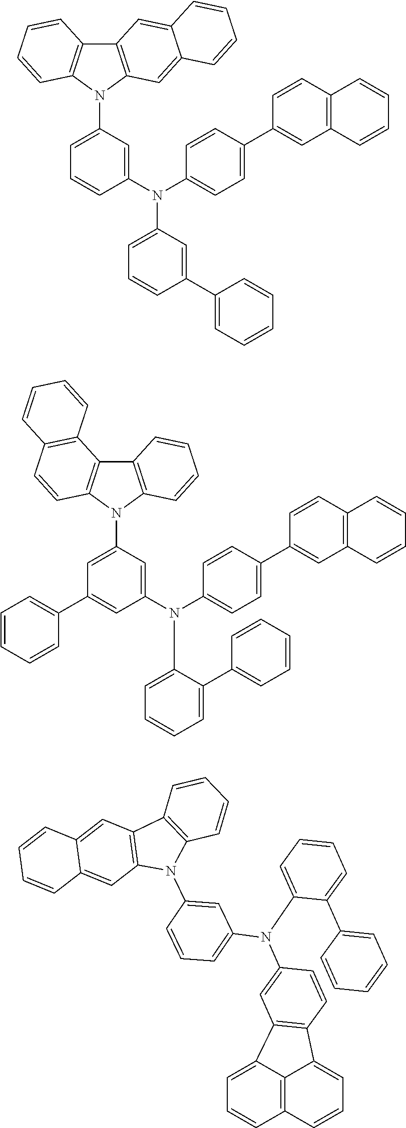 [SMILES: c1ccc(-c2cccc(N(c3ccc(-c4ccc5ccccc5c4)cc3)c3cccc(-n4c5ccccc5c5cc6ccccc6cc54)c3)c2)cc1 ; c1ccc(-c2cc(N(c3ccc(-c4ccc5ccccc5c4)cc3)c3ccccc3-c3ccccc3)cc(-n3c4ccccc4c4c5ccccc5ccc43)c2)cc1 ; c1ccc(-c2ccccc2N(c2cccc(-n3c4ccccc4c4cc5ccccc5cc43)c2)c2ccc3c(c2)-c2cccc4cccc-3c24)cc1]

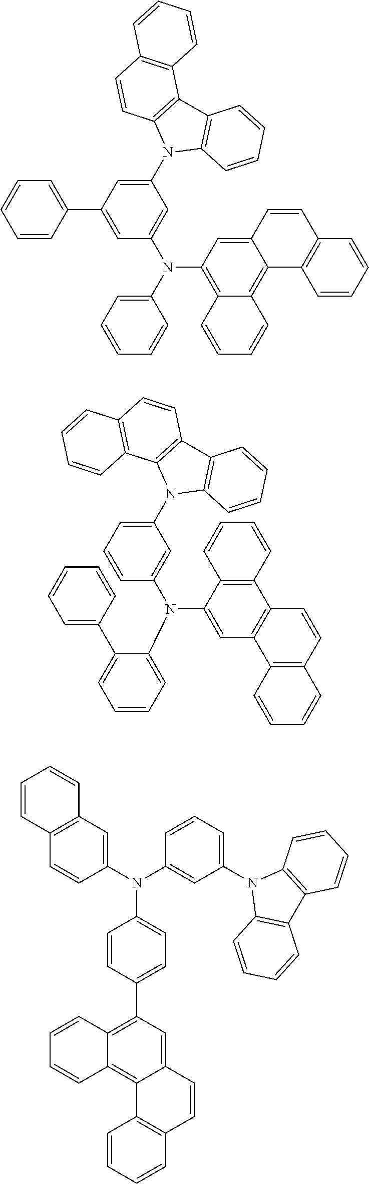

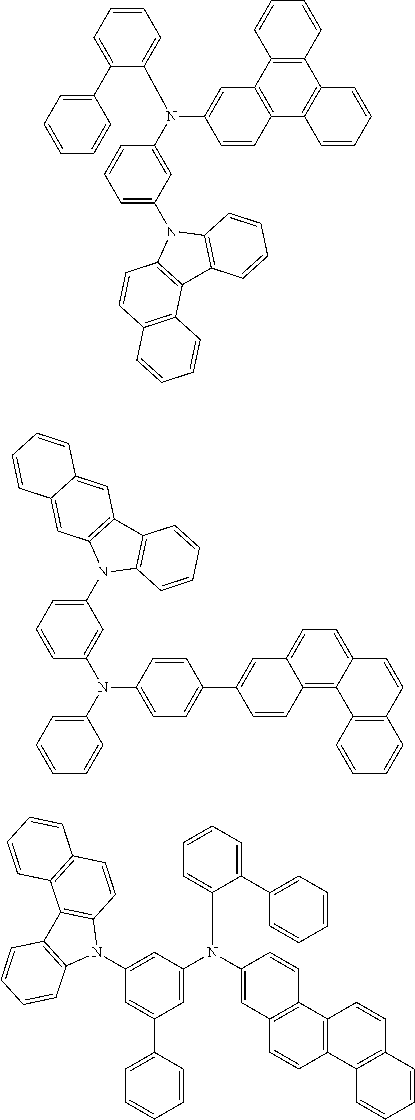 [SMILES: c1ccc(-c2ccccc2N(c2cccc(-n3c4ccccc4c4c5ccccc5ccc43)c2)c2ccc3c4ccccc4c4ccccc4c3c2)cc1 ; c1ccc(N(c2ccc(-c3ccc4c(ccc5ccc6ccccc6c54)c3)cc2)c2cccc(-n3c4ccccc4c4cc5ccccc5cc43)c2)cc1 ; c1ccc(-c2cc(N(c3ccc4c(ccc5c6ccccc6ccc45)c3)c3ccccc3-c3ccccc3)cc(-n3c4ccccc4c4c5ccccc5ccc43)c2)cc1]

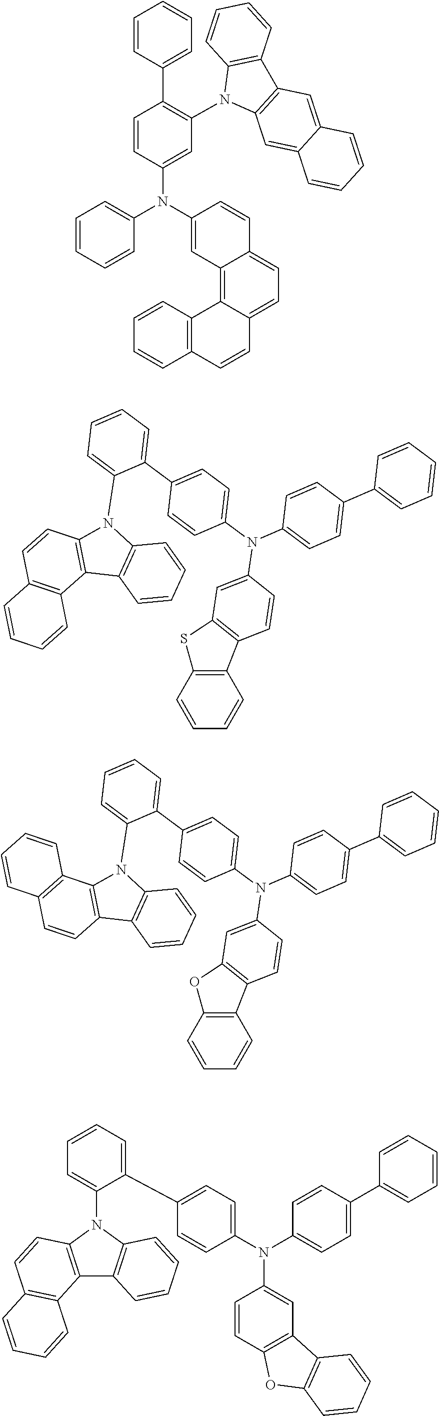 [SMILES: c1ccc(-c2ccc(N(c3ccccc3)c3ccc4ccc5ccc6ccccc6c5c4c3)cc2-n2c3ccccc3c3cc4ccccc4cc32)cc1 ; c1ccc(-c2ccc(N(c3ccc(-c4ccccc4-n4c5ccccc5c5c6ccccc6ccc54)cc3)c3ccc4c(c3)sc3ccccc34)cc2)cc1 ; c1ccc(-c2ccc(N(c3ccc(-c4ccccc4-n4c5ccccc5c5ccc6ccccc6c54)cc3)c3ccc4c(c3)oc3ccccc34)cc2)cc1 ; c1ccc(-c2ccc(N(c3ccc(-c4ccccc4-n4c5ccccc5c5c6ccccc6ccc54)cc3)c3ccc4oc5ccccc5c4c3)cc2)cc1]

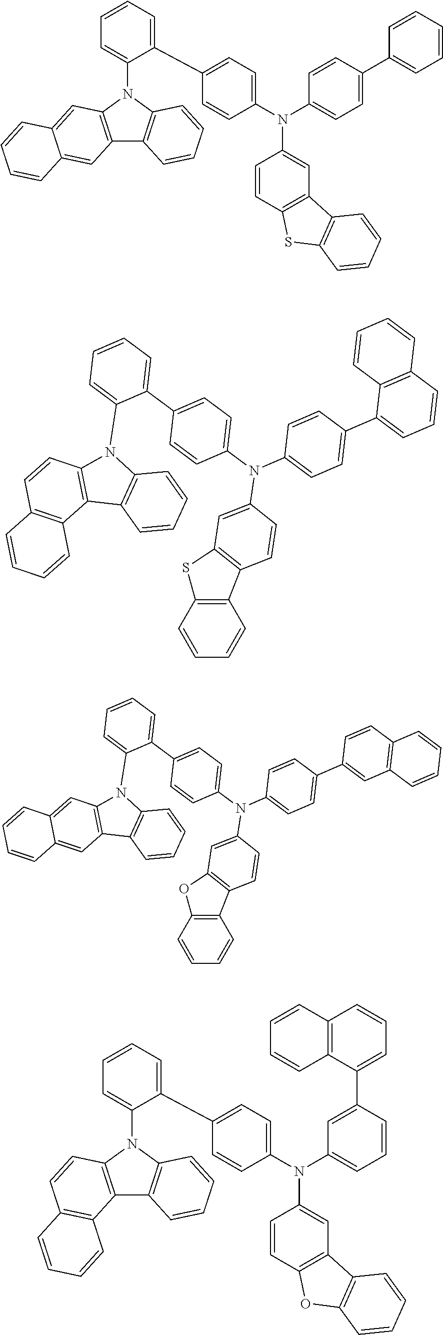 [SMILES: c1ccc(-c2ccc(N(c3ccc(-c4ccccc4-n4c5ccccc5c5cc6ccccc6cc54)cc3)c3ccc4sc5ccccc5c4c3)cc2)cc1 ; c1ccc(-n2c3ccccc3c3c4ccccc4ccc32)c(-c2ccc(N(c3ccc(-c4cccc5ccccc45)cc3)c3ccc4c(c3)sc3ccccc34)cc2)c1 ; c1ccc(-n2c3ccccc3c3cc4ccccc4cc32)c(-c2ccc(N(c3ccc(-c4ccc5ccccc5c4)cc3)c3ccc4c(c3)oc3ccccc34)cc2)c1 ; c1cc(-c2cccc3ccccc23)cc(N(c2ccc(-c3ccccc3-n3c4ccccc4c4c5ccccc5ccc43)cc2)c2ccc3oc4ccccc4c3c2)c1]

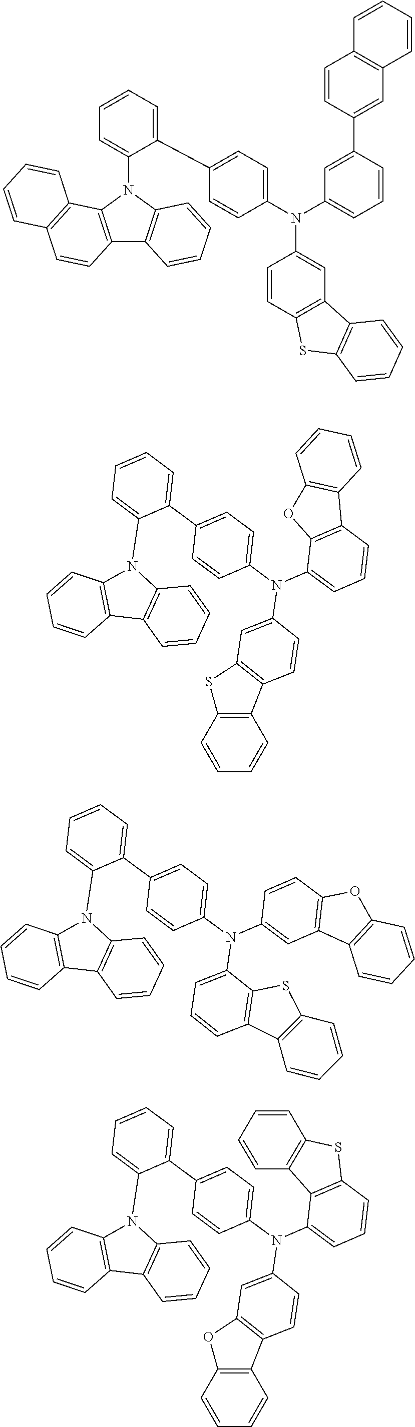

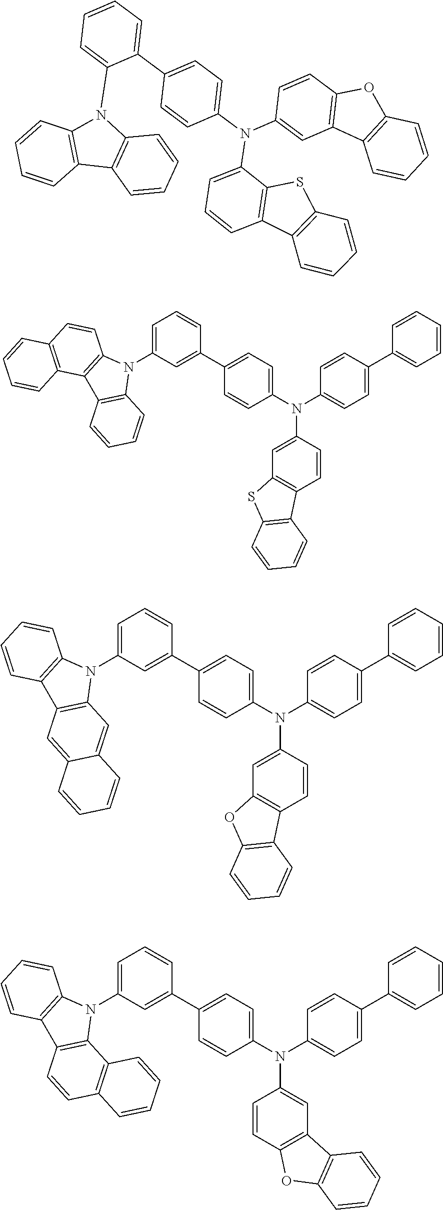 [SMILES: c1ccc(-n2c3ccccc3c3ccccc32)c(-c2ccc(N(c3ccc4oc5ccccc5c4c3)c3cccc4c3sc3ccccc34)cc2)c1 ; c1ccc(-c2ccc(N(c3ccc(-c4cccc(-n5c6ccccc6c6c7ccccc7ccc65)c4)cc3)c3ccc4c(c3)sc3ccccc34)cc2)cc1 ; c1ccc(-c2ccc(N(c3ccc(-c4cccc(-n5c6ccccc6c6cc7ccccc7cc65)c4)cc3)c3ccc4c(c3)oc3ccccc34)cc2)cc1 ; c1ccc(-c2ccc(N(c3ccc(-c4cccc(-n5c6ccccc6c6ccc7ccccc7c65)c4)cc3)c3ccc4oc5ccccc5c4c3)cc2)cc1]

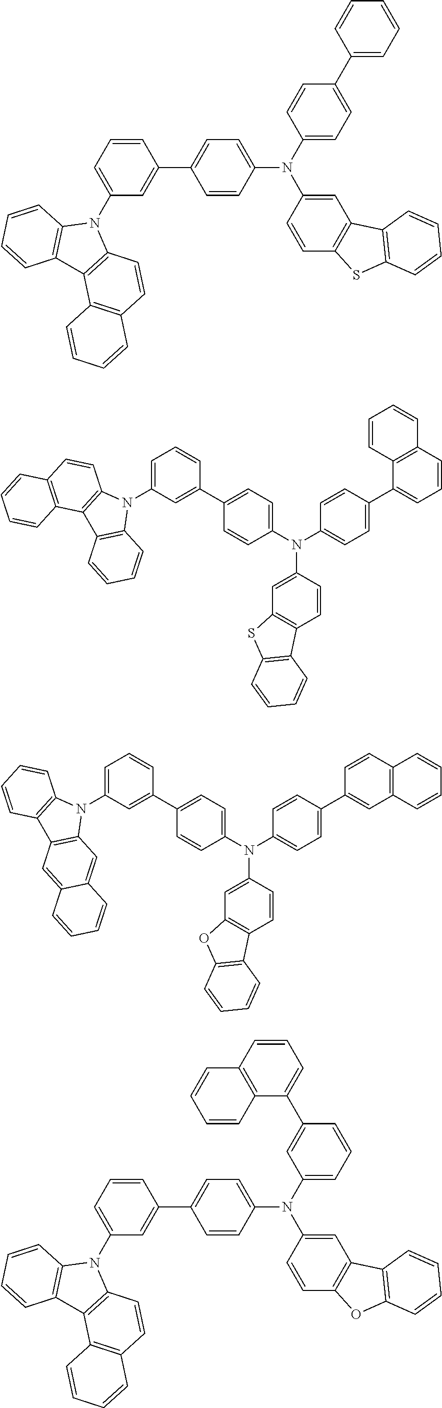

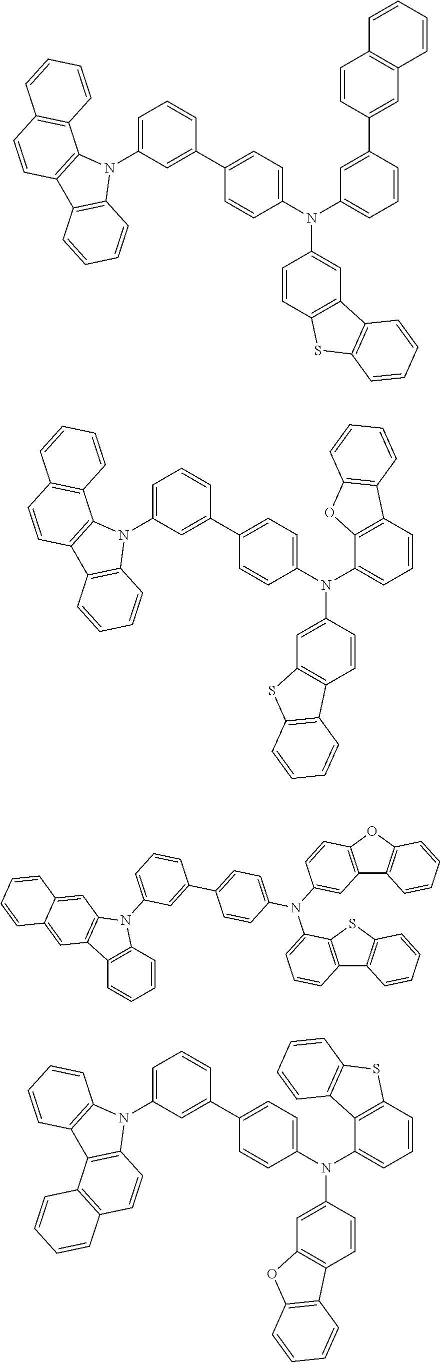 [SMILES: c1cc(-c2ccc3ccccc3c2)cc(N(c2ccc(-c3cccc(-n4c5ccccc5c5ccc6ccccc6c54)c3)cc2)c2ccc3sc4ccccc4c3c2)c1 ; c1cc(-c2ccc(N(c3ccc4c(c3)sc3ccccc34)c3cccc4c3oc3ccccc34)cc2)cc(-n2c3ccccc3c3ccc4ccccc4c32)c1 ; c1cc(-c2ccc(N(c3ccc4oc5ccccc5c4c3)c3cccc4c3sc3ccccc34)cc2)cc(-n2c3ccccc3c3cc4ccccc4cc32)c1 ; c1cc(-c2ccc(N(c3ccc4c(c3)oc3ccccc34)c3cccc4sc5ccccc5c34)cc2)cc(-n2c3ccccc3c3c4ccccc4ccc32)c1]

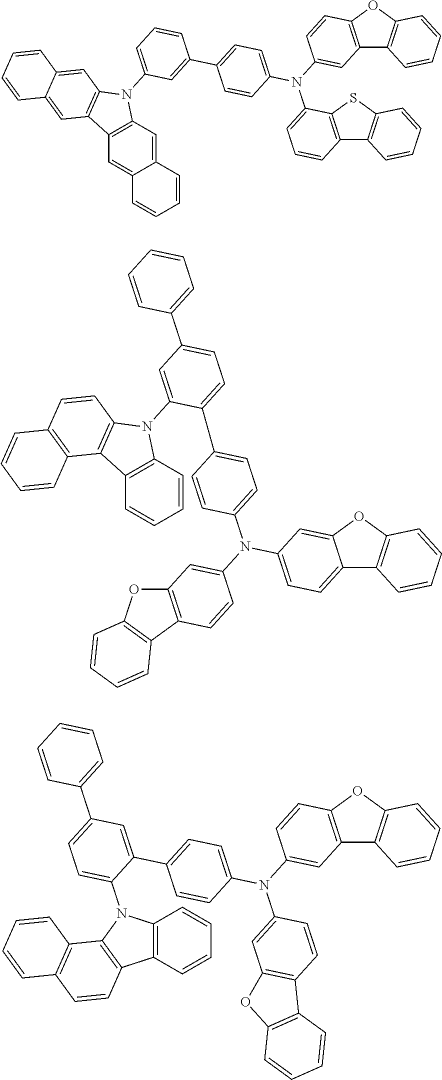

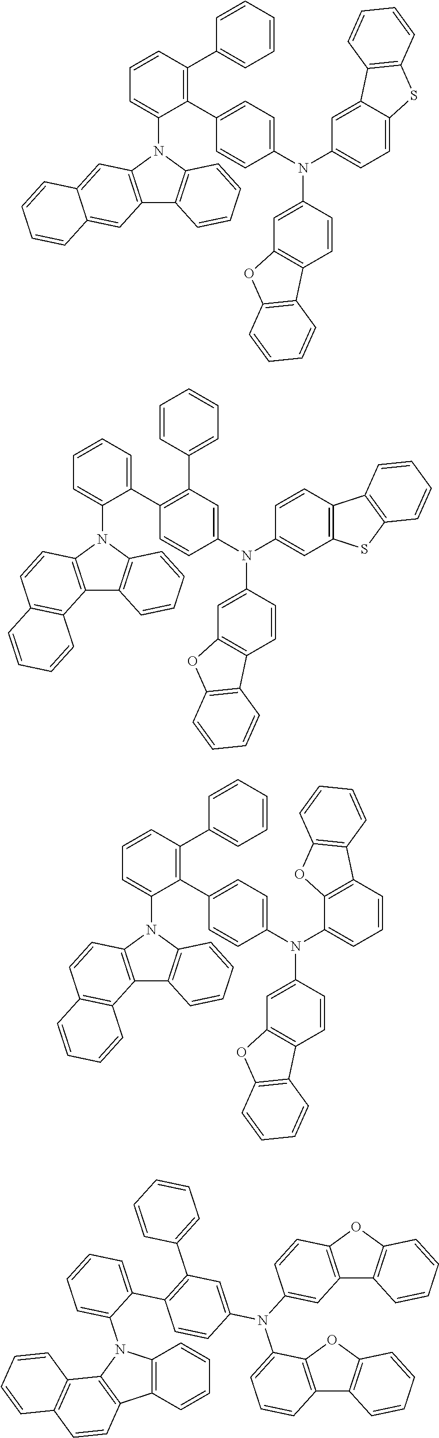

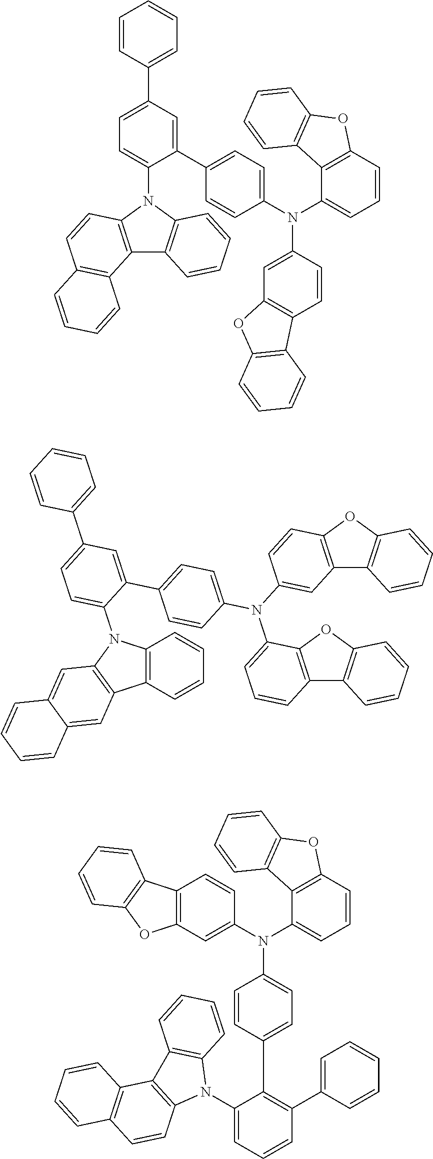 [SMILES: c1ccc(-c2ccc(-n3c4ccccc4c4c5ccccc5ccc43)c(-c3ccc(N(c4ccc5c(c4)oc4ccccc45)c4cccc5oc6ccccc6c45)cc3)c2)cc1 ; c1ccc(-c2ccc(-n3c4ccccc4c4cc5ccccc5cc43)c(-c3ccc(N(c4ccc5oc6ccccc6c5c4)c4cccc5c4oc4ccccc45)cc3)c2)cc1 ; c1ccc(-c2cccc(-n3c4ccccc4c4c5ccccc5ccc43)c2-c2ccc(N(c3ccc4c(c3)oc3ccccc34)c3cccc4oc5ccccc5c34)cc2)cc1]

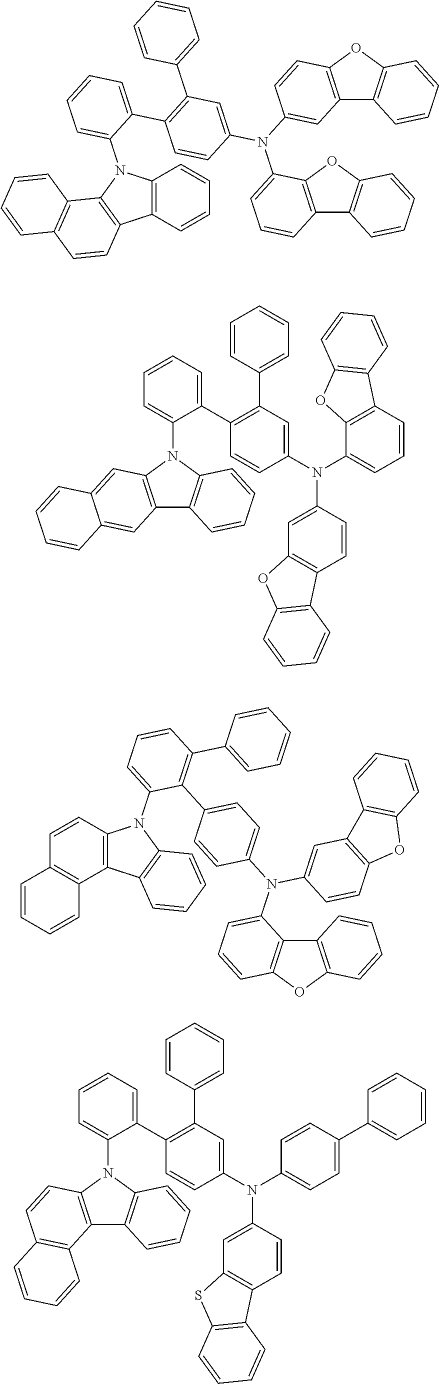

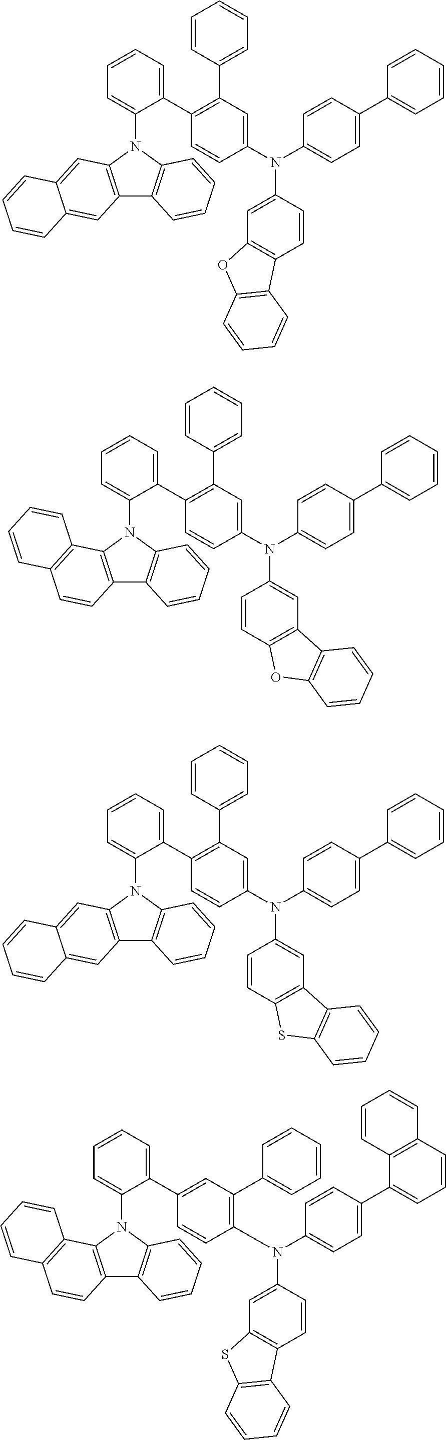

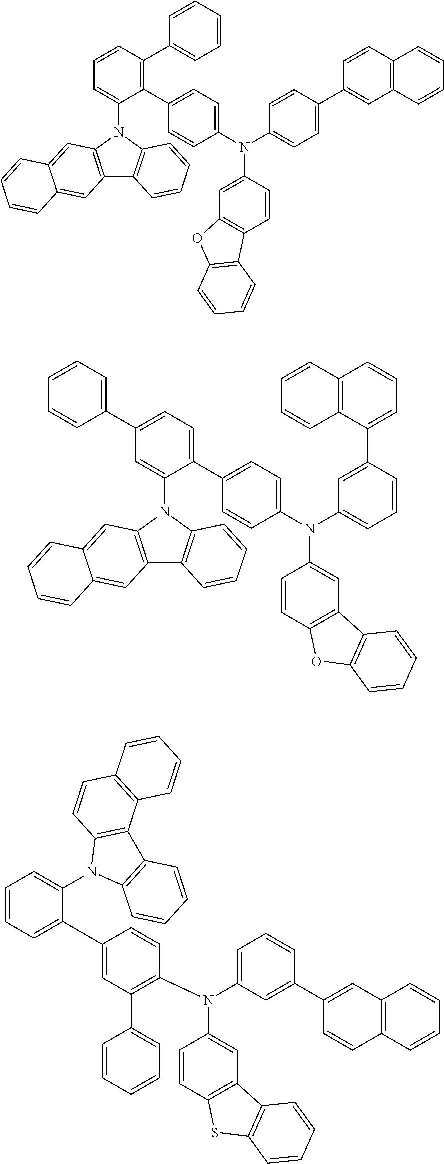 [SMILES: c1ccc(-c2cccc(-n3c4ccccc4c4cc5ccccc5cc43)c2-c2ccc(N(c3ccc(-c4ccc5ccccc5c4)cc3)c3ccc4c(c3)oc3ccccc34)cc2)cc1 ; c1ccc(-c2ccc(-c3ccc(N(c4cccc(-c5cccc6ccccc56)c4)c4ccc5oc6ccccc6c5c4)cc3)c(-n3c4ccccc4c4cc5ccccc5cc43)c2)cc1 ; c1ccc(-c2cc(-c3ccccc3-n3c4ccccc4c4c5ccccc5ccc43)ccc2N(c2cccc(-c3ccc4ccccc4c3)c2)c2ccc3sc4ccccc4c3c2)cc1]

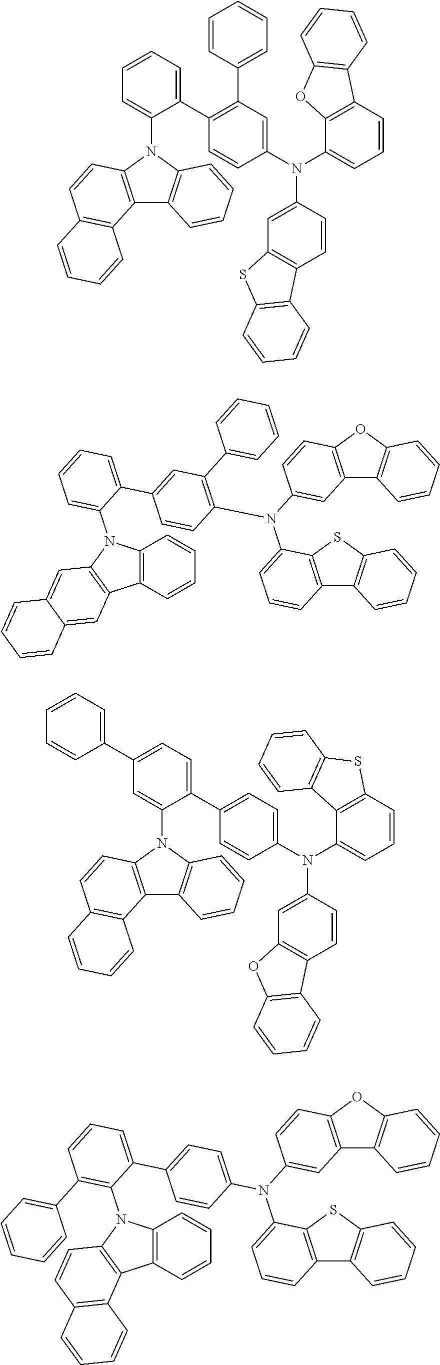

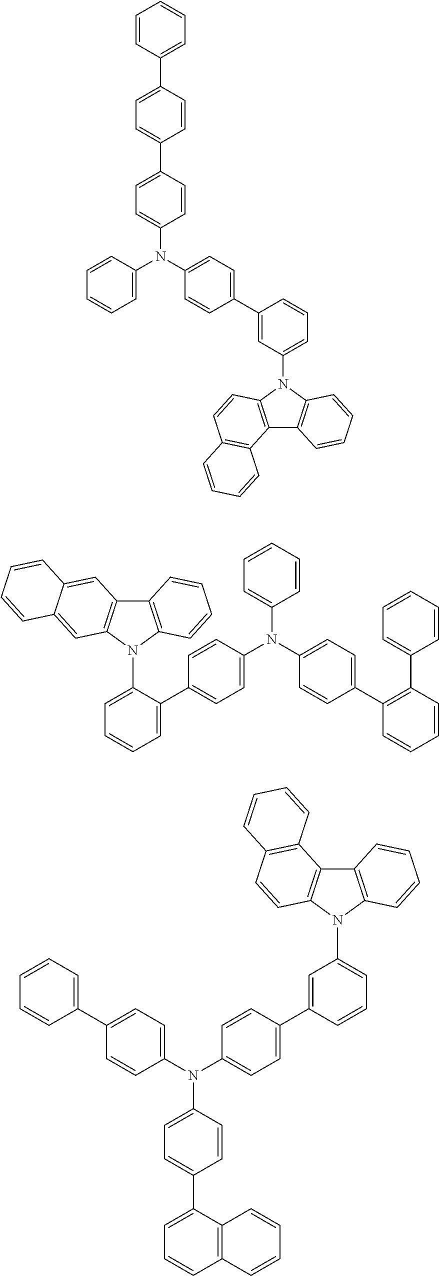 [SMILES: c1ccc(-c2ccc(-c3ccc(N(c4ccccc4)c4ccc(-c5cccc(-n6c7ccccc7c7c8ccccc8ccc76)c5)cc4)cc3)cc2)cc1 ; c1ccc(-c2ccccc2-c2ccc(N(c3ccccc3)c3ccc(-c4ccccc4-n4c5ccccc5c5cc6ccccc6cc54)cc3)cc2)cc1 ; c1ccc(-c2ccc(N(c3ccc(-c4cccc(-n5c6ccccc6c6c7ccccc7ccc65)c4)cc3)c3ccc(-c4cccc5ccccc45)cc3)cc2)cc1]

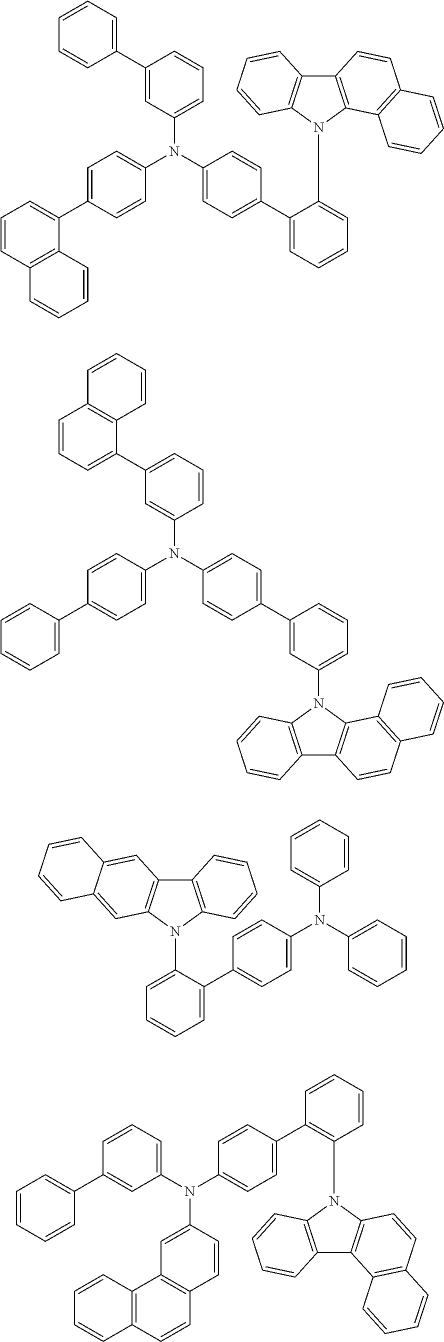 [SMILES: c1ccc(-c2cccc(N(c3ccc(-c4ccccc4-n4c5ccccc5c5ccc6ccccc6c54)cc3)c3ccc(-c4cccc5ccccc45)cc3)c2)cc1 ; c1ccc(-c2ccc(N(c3ccc(-c4cccc(-n5c6ccccc6c6ccc7ccccc7c65)c4)cc3)c3cccc(-c4cccc5ccccc45)c3)cc2)cc1 ; c1ccc(N(c2ccccc2)c2ccc(-c3ccccc3-n3c4ccccc4c4cc5ccccc5cc43)cc2)cc1 ; c1ccc(-c2cccc(N(c3ccc(-c4ccccc4-n4c5ccccc5c5c6ccccc6ccc54)cc3)c3ccc4ccc5ccccc5c4c3)c2)cc1]

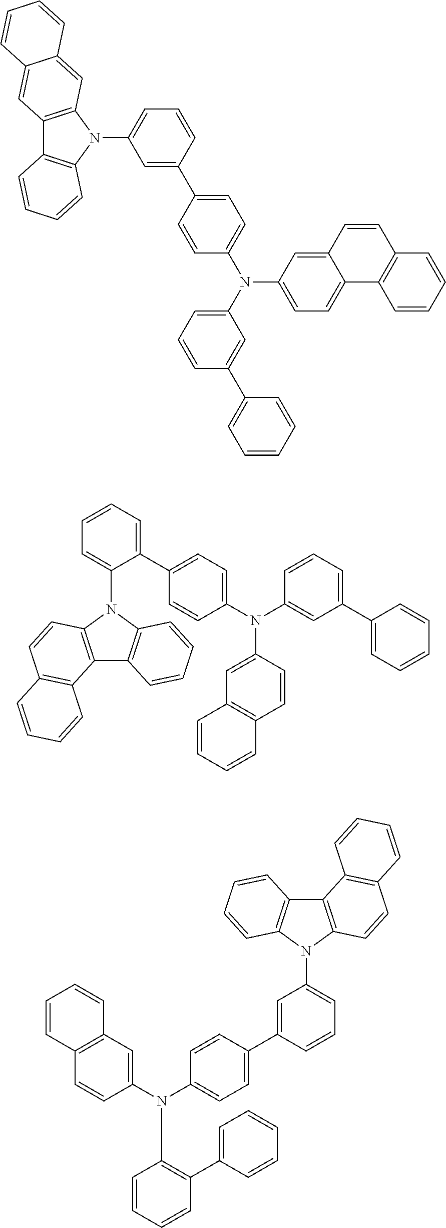 [SMILES: c1ccc(-c2cccc(N(c3ccc(-c4cccc(-n5c6ccccc6c6cc7ccccc7cc65)c4)cc3)c3ccc4c(ccc5ccccc54)c3)c2)cc1 ; c1ccc(-c2cccc(N(c3ccc(-c4ccccc4-n4c5ccccc5c5c6ccccc6ccc54)cc3)c3ccc4ccccc4c3)c2)cc1 ; c1ccc(-c2ccccc2N(c2ccc(-c3cccc(-n4c5ccccc5c5c6ccccc6ccc54)c3)cc2)c2ccc3ccccc3c2)cc1]

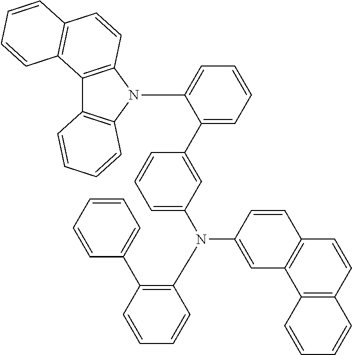 [SMILES: c1ccc(-c2ccccc2N(c2cccc(-c3ccccc3-n3c4ccccc4c4c5ccccc5ccc43)c2)c2ccc3ccc4ccccc4c3c2)cc1]

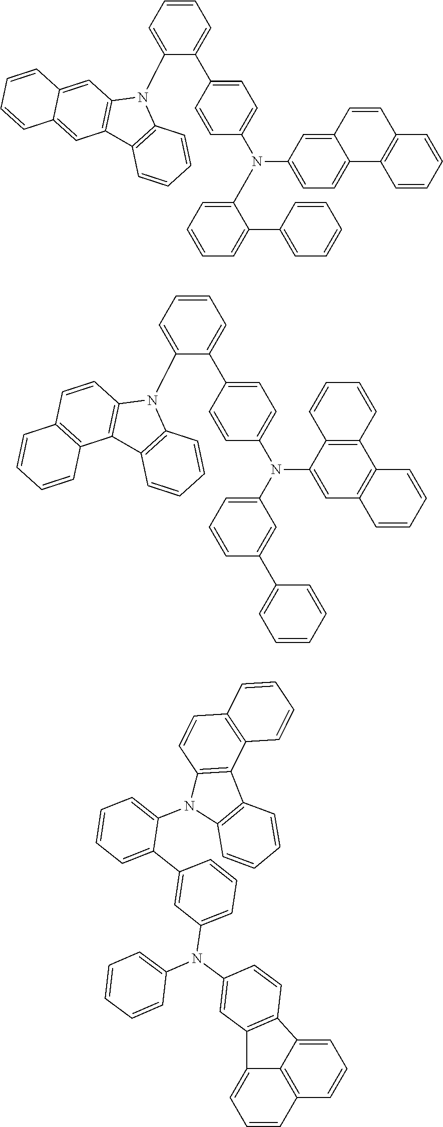

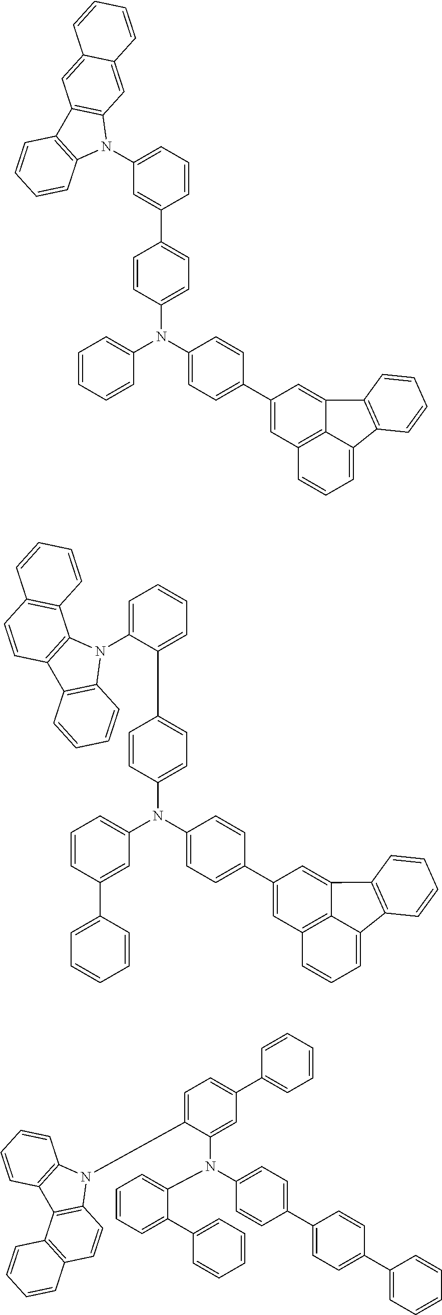 [SMILES: c1ccc(N(c2ccc(-c3cccc(-n4c5ccccc5c5cc6ccccc6cc54)c3)cc2)c2ccc(-c3cc4c5c(cccc5c3)-c3ccccc3-4)cc2)cc1 ; c1ccc(-c2cccc(N(c3ccc(-c4cc5c6c(cccc6c4)-c4ccccc4-5)cc3)c3ccc(-c4ccccc4-n4c5ccccc5c5ccc6ccccc6c54)cc3)c2)cc1 ; c1ccc(-c2ccc(-c3ccc(N(c4ccccc4-c4ccccc4)c4cc(-c5ccccc5)ccc4-n4c5ccccc5c5c6ccccc6ccc54)cc3)cc2)cc1]

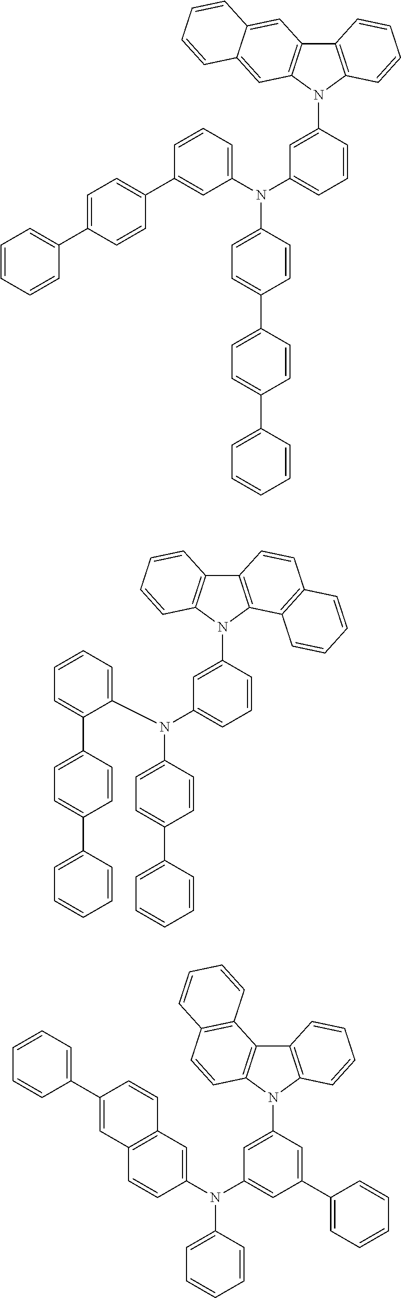 [SMILES: c1ccc(-c2ccc(-c3ccc(N(c4cccc(-c5ccc(-c6ccccc6)cc5)c4)c4cccc(-n5c6ccccc6c6cc7ccccc7cc65)c4)cc3)cc2)cc1 ; c1ccc(-c2ccc(-c3ccccc3N(c3ccc(-c4ccccc4)cc3)c3cccc(-n4c5ccccc5c5ccc6ccccc6c54)c3)cc2)cc1 ; c1ccc(-c2cc(N(c3ccccc3)c3ccc4cc(-c5ccccc5)ccc4c3)cc(-n3c4ccccc4c4c5ccccc5ccc43)c2)cc1]

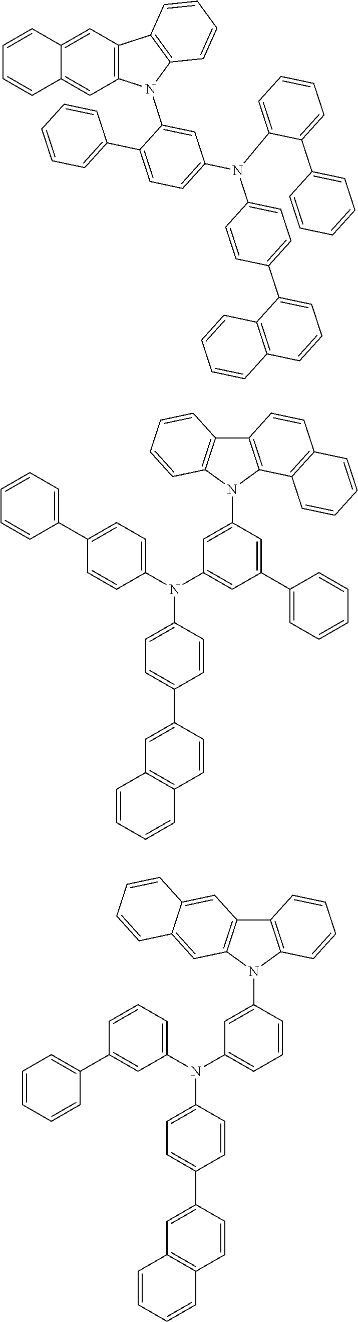

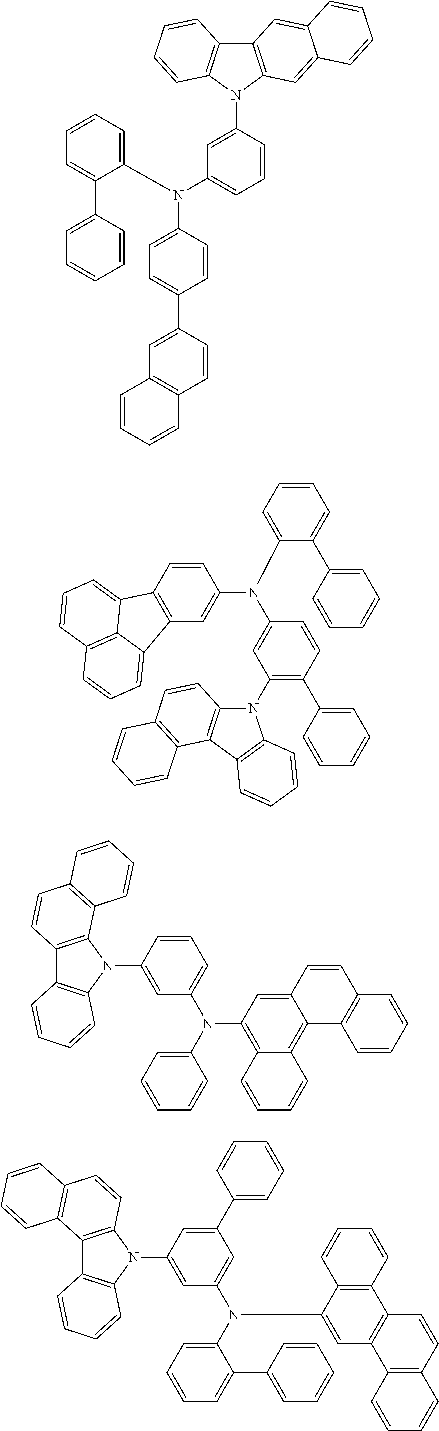

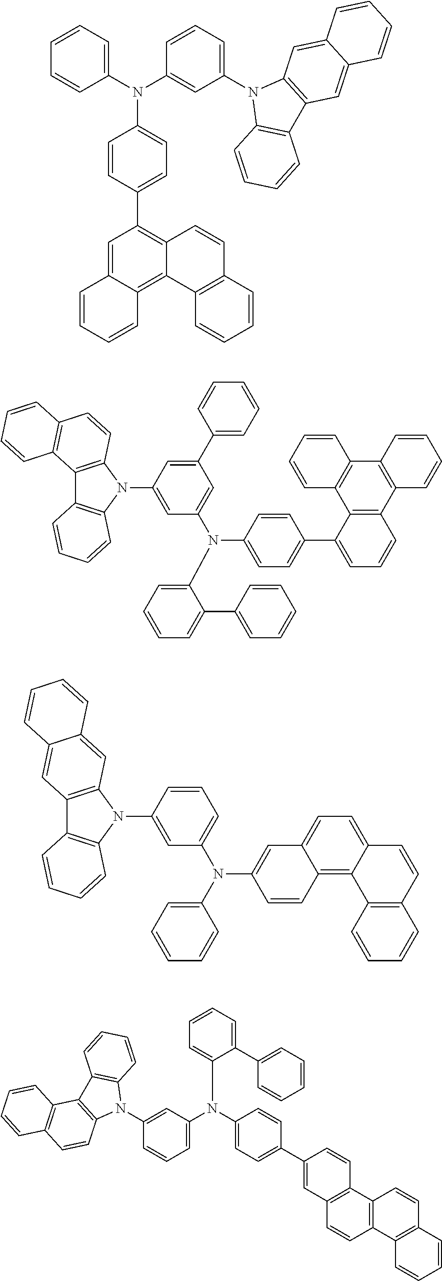

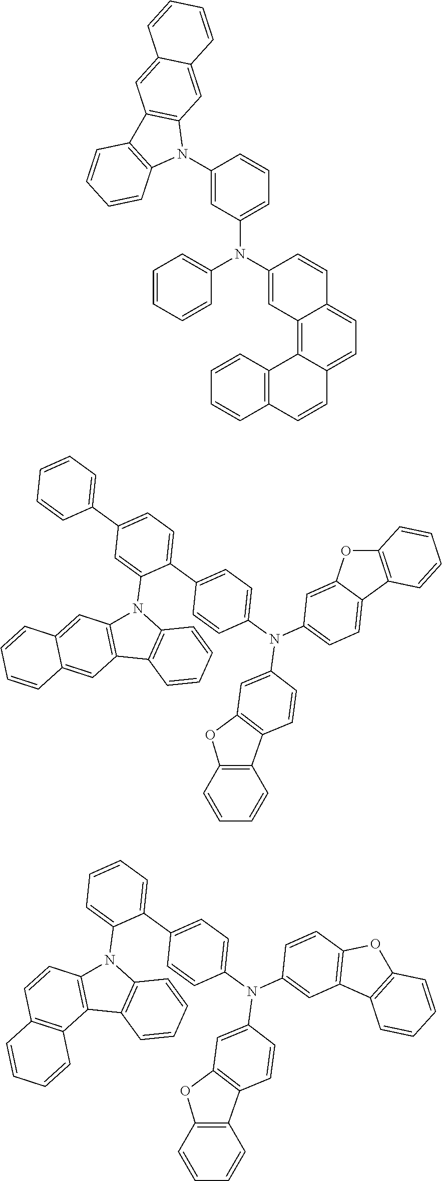 [SMILES: c1ccc(N(c2cccc(-n3c4ccccc4c4cc5ccccc5cc43)c2)c2ccc3ccc4ccc5ccccc5c4c3c2)cc1 ; c1ccc(-c2ccc(-c3ccc(N(c4ccc5c(c4)oc4ccccc45)c4ccc5c(c4)oc4ccccc45)cc3)c(-n3c4ccccc4c4cc5ccccc5cc43)c2)cc1 ; c1ccc(-n2c3ccccc3c3c4ccccc4ccc32)c(-c2ccc(N(c3ccc4c(c3)oc3ccccc34)c3ccc4oc5ccccc5c4c3)cc2)c1]

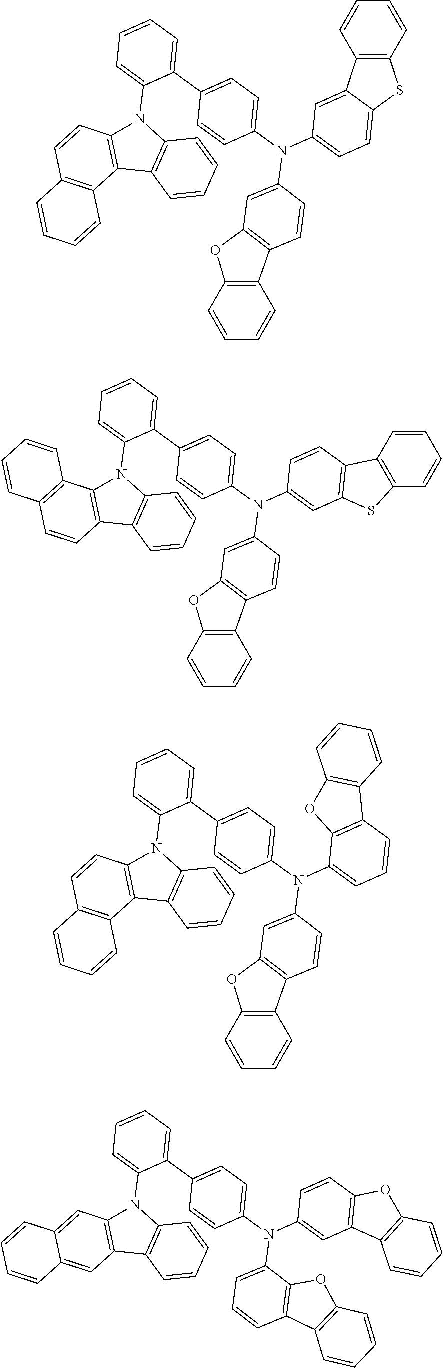 [SMILES: c1ccc(-n2c3ccccc3c3c4ccccc4ccc32)c(-c2ccc(N(c3ccc4c(c3)oc3ccccc34)c3ccc4sc5ccccc5c4c3)cc2)c1 ; c1ccc(-n2c3ccccc3c3ccc4ccccc4c32)c(-c2ccc(N(c3ccc4c(c3)oc3ccccc34)c3ccc4c(c3)sc3ccccc34)cc2)c1 ; c1ccc(-n2c3ccccc3c3c4ccccc4ccc32)c(-c2ccc(N(c3ccc4c(c3)oc3ccccc34)c3cccc4c3oc3ccccc34)cc2)c1 ; c1ccc(-n2c3ccccc3c3cc4ccccc4cc32)c(-c2ccc(N(c3ccc4oc5ccccc5c4c3)c3cccc4c3oc3ccccc34)cc2)c1]

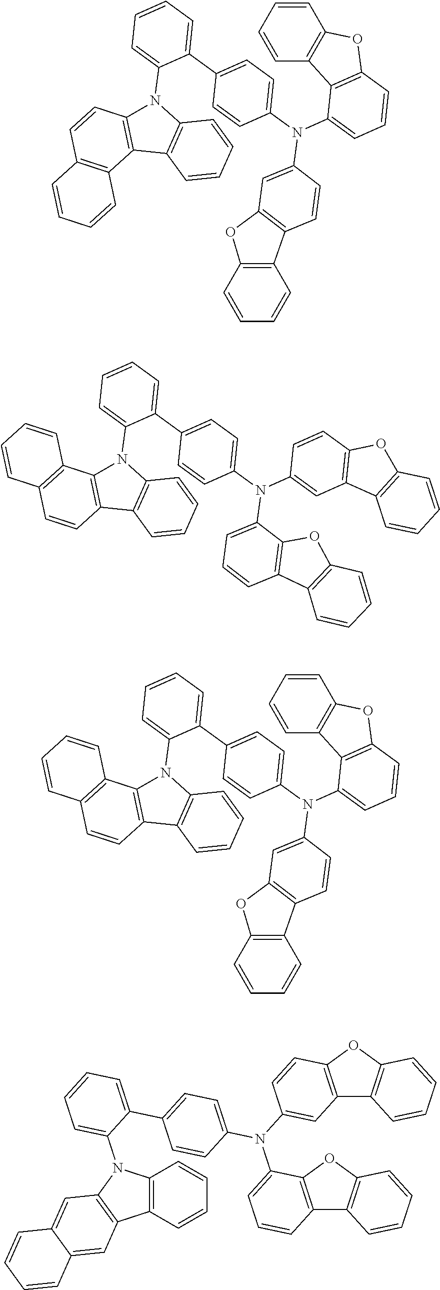 [SMILES: c1ccc(-n2c3ccccc3c3c4ccccc4ccc32)c(-c2ccc(N(c3ccc4c(c3)oc3ccccc34)c3cccc4oc5ccccc5c34)cc2)c1 ; c1ccc(-n2c3ccccc3c3ccc4ccccc4c32)c(-c2ccc(N(c3ccc4oc5ccccc5c4c3)c3cccc4c3oc3ccccc34)cc2)c1 ; c1ccc(-n2c3ccccc3c3ccc4ccccc4c32)c(-c2ccc(N(c3ccc4c(c3)oc3ccccc34)c3cccc4oc5ccccc5c34)cc2)c1 ; c1ccc(-n2c3ccccc3c3cc4ccccc4cc32)c(-c2ccc(N(c3ccc4oc5ccccc5c4c3)c3cccc4c3oc3ccccc34)cc2)c1]

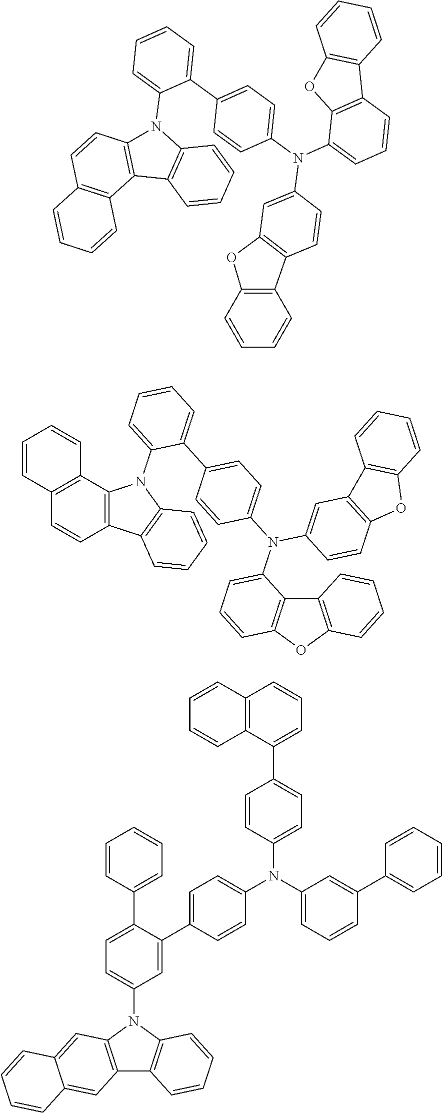

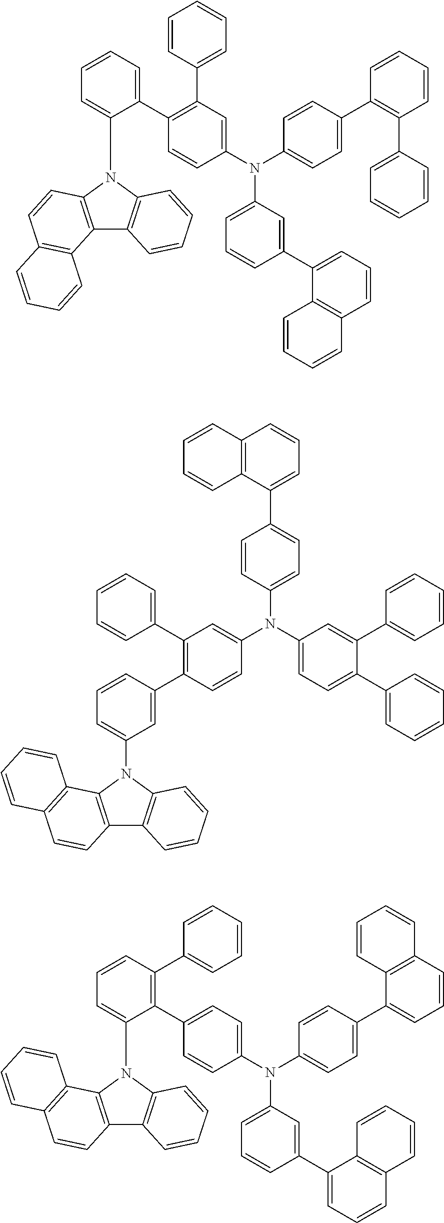 [SMILES: c1ccc(-c2ccccc2-c2ccc(N(c3cccc(-c4cccc5ccccc45)c3)c3ccc(-c4ccccc4-n4c5ccccc5c5c6ccccc6ccc54)c(-c4ccccc4)c3)cc2)cc1 ; c1ccc(-c2ccc(N(c3ccc(-c4cccc5ccccc45)cc3)c3ccc(-c4cccc(-n5c6ccccc6c6ccc7ccccc7c65)c4)c(-c4ccccc4)c3)cc2-c2ccccc2)cc1 ; c1ccc(-c2cccc(-n3c4ccccc4c4ccc5ccccc5c43)c2-c2ccc(N(c3ccc(-c4cccc5ccccc45)cc3)c3cccc(-c4cccc5ccccc45)c3)cc2)cc1]

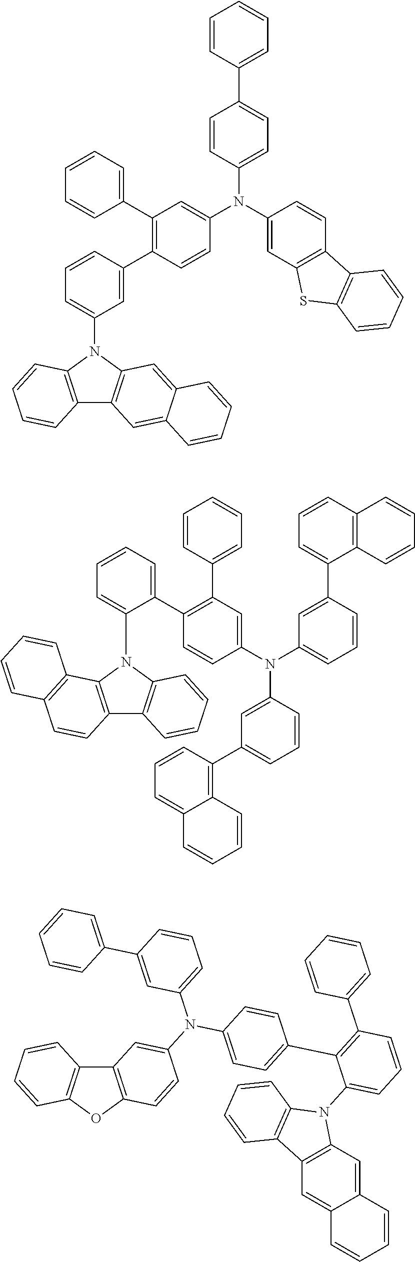 [SMILES: c1ccc(-c2ccc(N(c3ccc(-c4cccc(-n5c6ccccc6c6cc7ccccc7cc65)c4)c(-c4ccccc4)c3)c3ccc4c(c3)sc3ccccc34)cc2)cc1 ; c1ccc(-c2cc(N(c3cccc(-c4cccc5ccccc45)c3)c3cccc(-c4cccc5ccccc45)c3)ccc2-c2ccccc2-n2c3ccccc3c3ccc4ccccc4c32)cc1 ; c1ccc(-c2cccc(N(c3ccc(-c4c(-c5ccccc5)cccc4-n4c5ccccc5c5cc6ccccc6cc54)cc3)c3ccc4oc5ccccc5c4c3)c2)cc1]

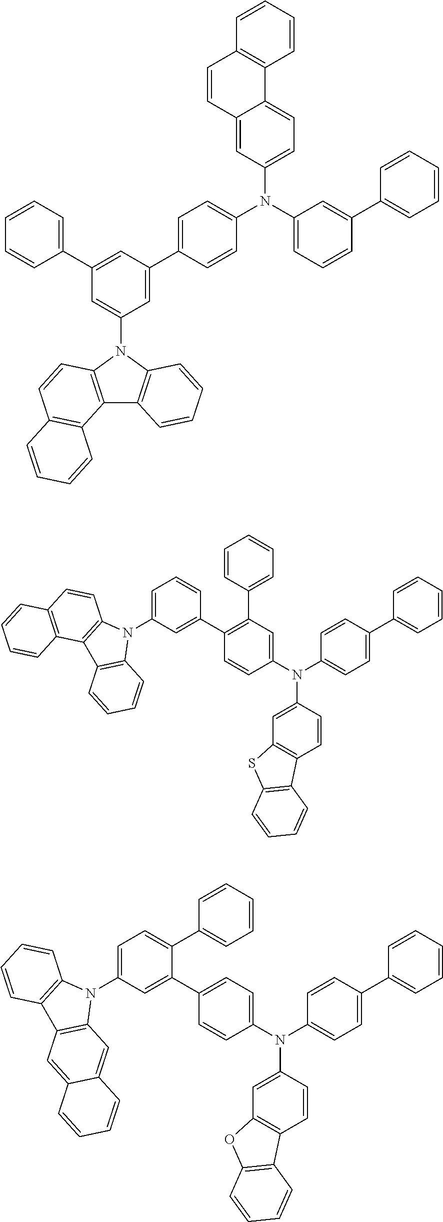 [SMILES: c1ccc(-c2cccc(N(c3ccc(-c4cc(-c5ccccc5)cc(-n5c6ccccc6c6c7ccccc7ccc65)c4)cc3)c3ccc4c(ccc5ccccc54)c3)c2)cc1 ; c1ccc(-c2ccc(N(c3ccc(-c4cccc(-n5c6ccccc6c6c7ccccc7ccc65)c4)c(-c4ccccc4)c3)c3ccc4c(c3)sc3ccccc34)cc2)cc1 ; c1ccc(-c2ccc(N(c3ccc(-c4cc(-n5c6ccccc6c6cc7ccccc7cc65)ccc4-c4ccccc4)cc3)c3ccc4c(c3)oc3ccccc34)cc2)cc1]

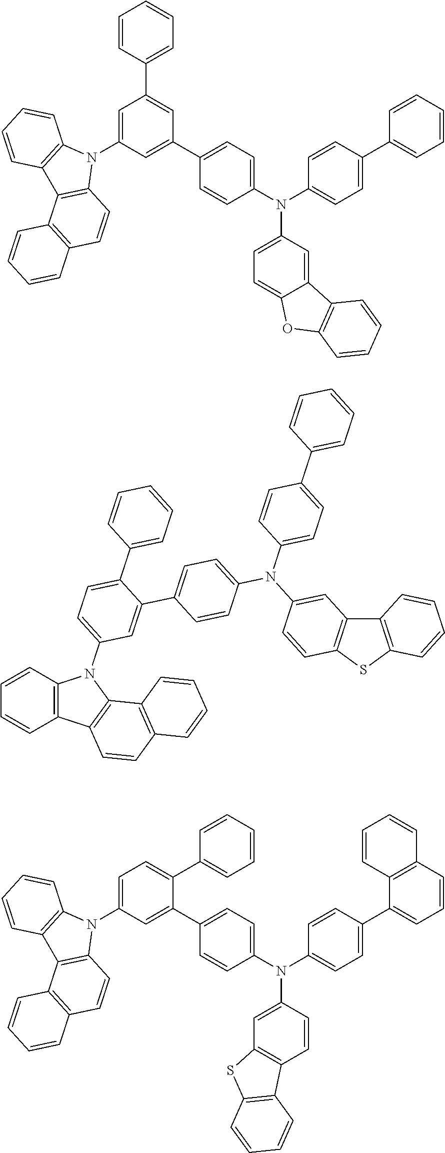

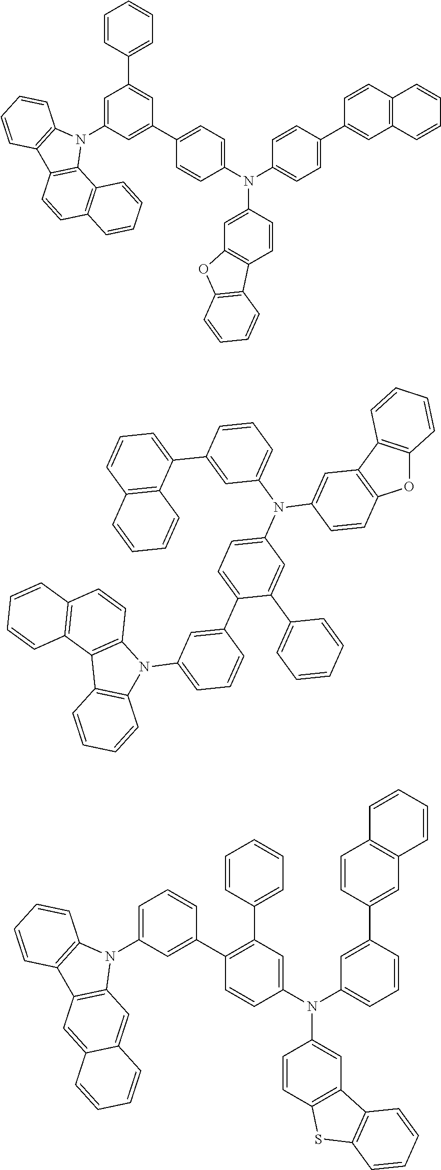 [SMILES: c1ccc(-c2cc(-c3ccc(N(c4ccc(-c5ccc6ccccc6c5)cc4)c4ccc5c(c4)oc4ccccc45)cc3)cc(-n3c4ccccc4c4ccc5ccccc5c43)c2)cc1 ; c1ccc(-c2cc(N(c3cccc(-c4cccc5ccccc45)c3)c3ccc4oc5ccccc5c4c3)ccc2-c2cccc(-n3c4ccccc4c4c5ccccc5ccc43)c2)cc1 ; c1ccc(-c2cc(N(c3cccc(-c4ccc5ccccc5c4)c3)c3ccc4sc5ccccc5c4c3)ccc2-c2cccc(-n3c4ccccc4c4cc5ccccc5cc43)c2)cc1]

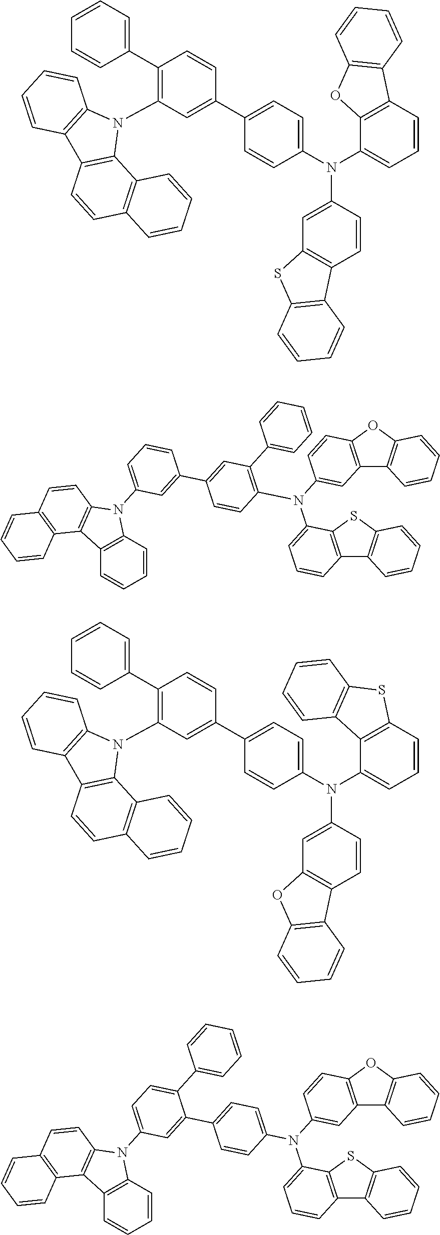

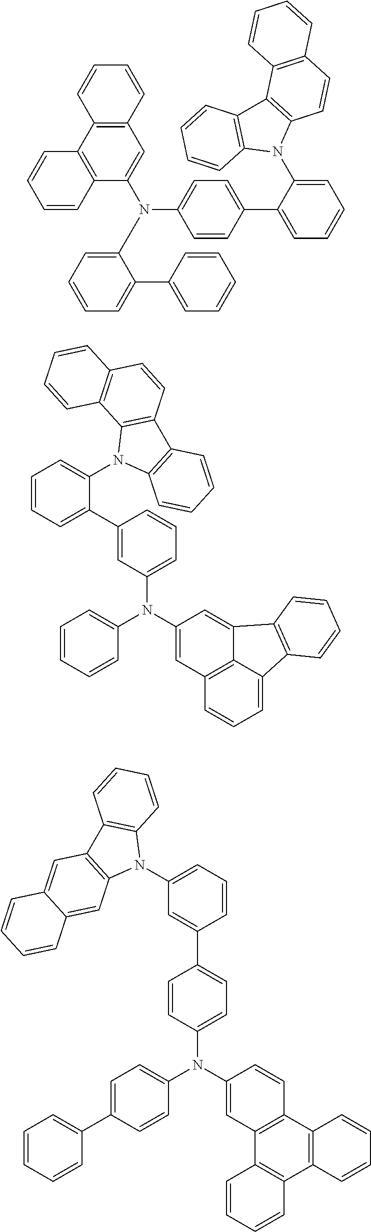

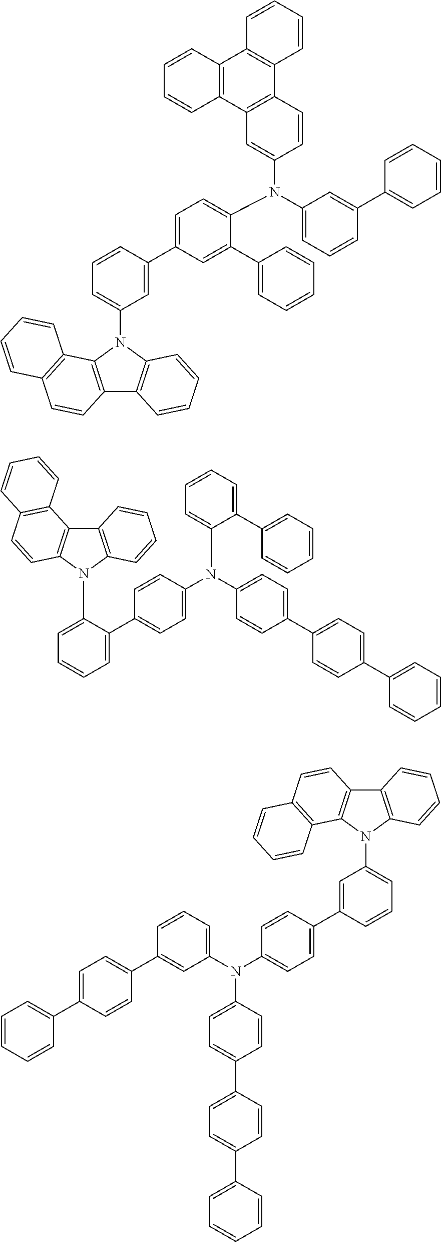 [SMILES: c1ccc(-c2cccc(N(c3ccc4c5ccccc5c5ccccc5c4c3)c3ccc(-c4cccc(-n5c6ccccc6c6ccc7ccccc7c65)c4)cc3-c3ccccc3)c2)cc1 ; c1ccc(-c2ccc(-c3ccc(N(c4ccc(-c5ccccc5-n5c6ccccc6c6c7ccccc7ccc65)cc4)c4ccccc4-c4ccccc4)cc3)cc2)cc1 ; c1ccc(-c2ccc(-c3ccc(N(c4ccc(-c5cccc(-n6c7ccccc7c7ccc8ccccc8c76)c5)cc4)c4cccc(-c5ccc(-c6ccccc6)cc5)c4)cc3)cc2)cc1]

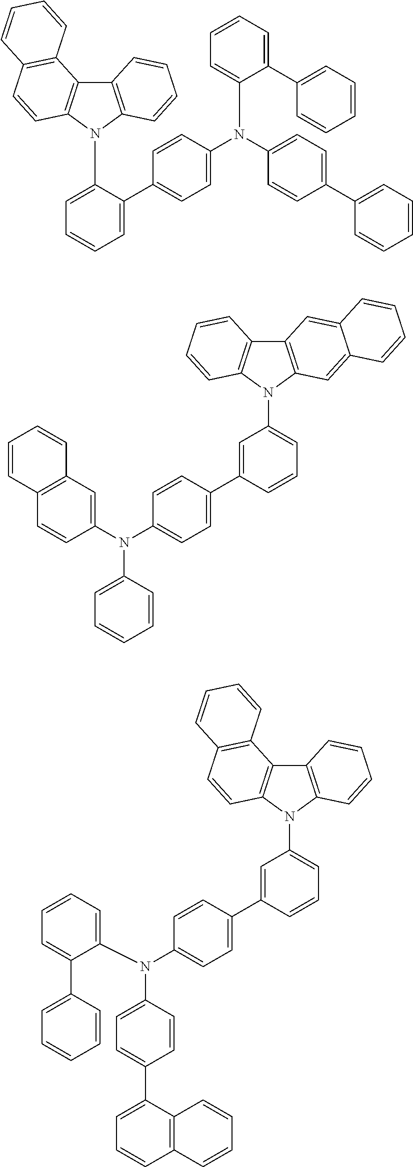 [SMILES: c1ccc(-c2ccc(N(c3ccc(-c4ccccc4-n4c5ccccc5c5c6ccccc6ccc54)cc3)c3ccccc3-c3ccccc3)cc2)cc1 ; c1ccc(N(c2ccc(-c3cccc(-n4c5ccccc5c5cc6ccccc6cc54)c3)cc2)c2ccc3ccccc3c2)cc1 ; c1ccc(-c2ccccc2N(c2ccc(-c3cccc(-n4c5ccccc5c5c6ccccc6ccc54)c3)cc2)c2ccc(-c3cccc4ccccc34)cc2)cc1]

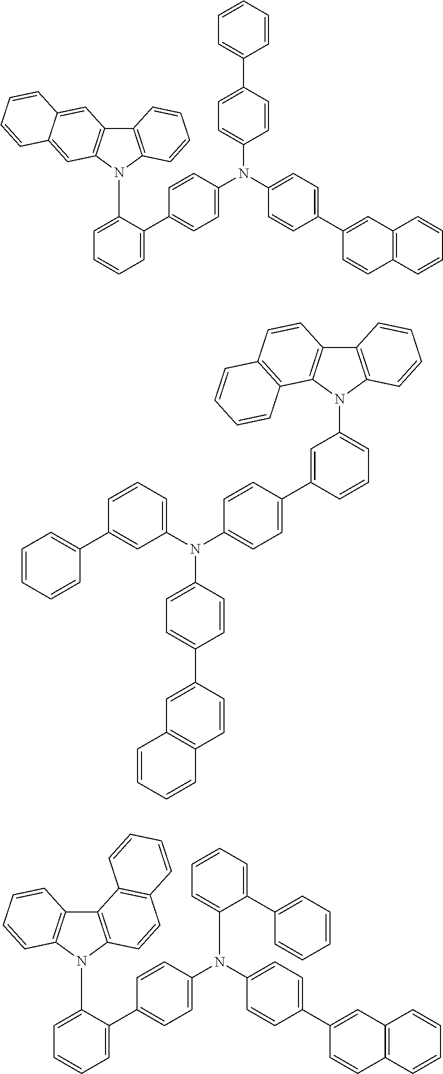

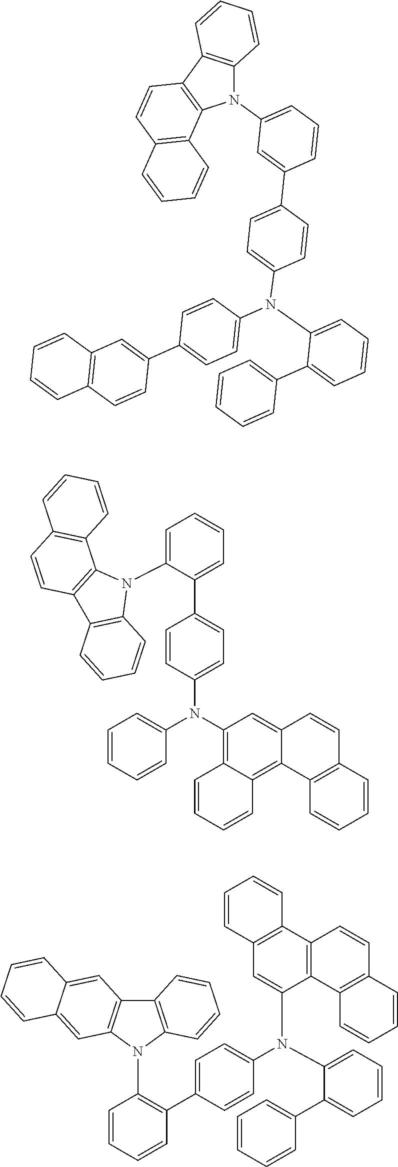 [SMILES: c1ccc(-c2ccccc2N(c2ccc(-c3cccc(-n4c5ccccc5c5ccc6ccccc6c54)c3)cc2)c2ccc(-c3ccc4ccccc4c3)cc2)cc1 ; c1ccc(N(c2ccc(-c3ccccc3-n3c4ccccc4c4ccc5ccccc5c43)cc2)c2cc3ccc4ccccc4c3c3ccccc23)cc1 ; c1ccc(-c2ccccc2N(c2ccc(-c3ccccc3-n3c4ccccc4c4cc5ccccc5cc43)cc2)c2cc3ccccc3c3ccc4ccccc4c23)cc1]

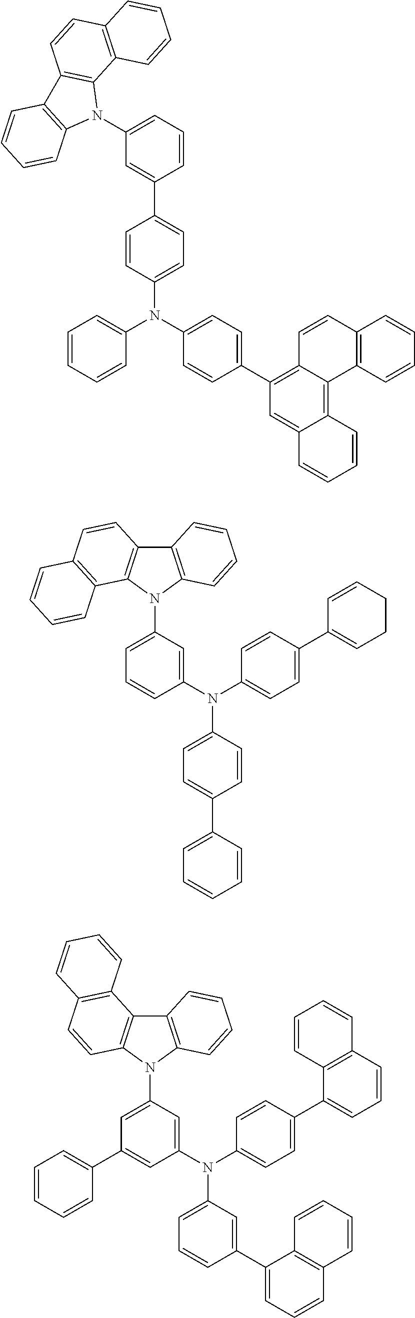 [SMILES: c1ccc(N(c2ccc(-c3cccc(-n4c5ccccc5c5ccc6ccccc6c54)c3)cc2)c2ccc(-c3cc4ccccc4c4c3ccc3ccccc34)cc2)cc1 ; C1=CC(c2ccc(N(c3ccc(-c4ccccc4)cc3)c3cccc(-n4c5ccccc5c5ccc6ccccc6c54)c3)cc2)=CCC1 ; c1ccc(-c2cc(N(c3ccc(-c4cccc5ccccc45)cc3)c3cccc(-c4cccc5ccccc45)c3)cc(-n3c4ccccc4c4c5ccccc5ccc43)c2)cc1]

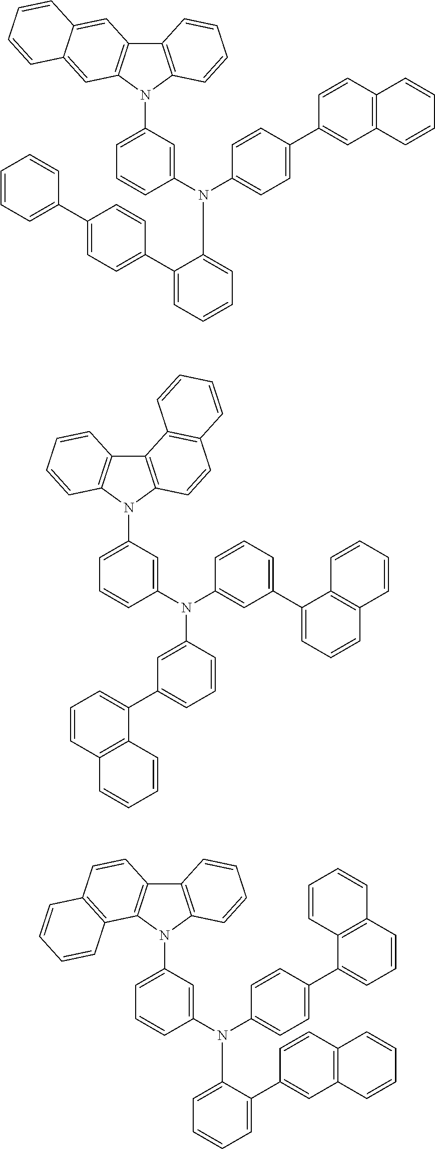 [SMILES: c1ccc(-c2ccc(-c3ccccc3N(c3ccc(-c4ccc5ccccc5c4)cc3)c3cccc(-n4c5ccccc5c5cc6ccccc6cc54)c3)cc2)cc1 ; c1cc(-c2cccc3ccccc23)cc(N(c2cccc(-c3cccc4ccccc34)c2)c2cccc(-n3c4ccccc4c4c5ccccc5ccc43)c2)c1 ; c1cc(N(c2ccc(-c3cccc4ccccc34)cc2)c2ccccc2-c2ccc3ccccc3c2)cc(-n2c3ccccc3c3ccc4ccccc4c32)c1]

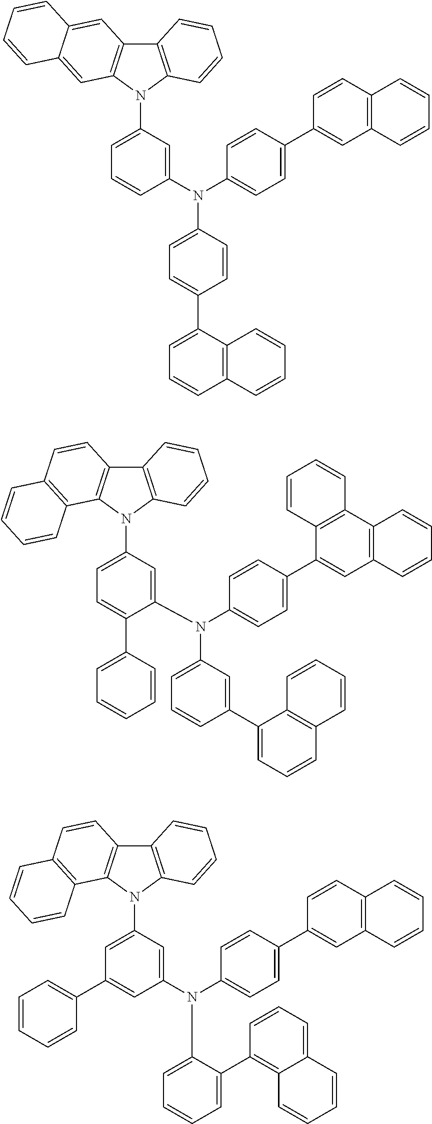 [SMILES: c1cc(N(c2ccc(-c3ccc4ccccc4c3)cc2)c2ccc(-c3cccc4ccccc34)cc2)cc(-n2c3ccccc3c3cc4ccccc4cc32)c1 ; c1ccc(-c2ccc(-n3c4ccccc4c4ccc5ccccc5c43)cc2N(c2ccc(-c3cc4ccccc4c4ccccc34)cc2)c2cccc(-c3cccc4ccccc34)c2)cc1 ; c1ccc(-c2cc(N(c3ccc(-c4ccc5ccccc5c4)cc3)c3ccccc3-c3cccc4ccccc34)cc(-n3c4ccccc4c4ccc5ccccc5c43)c2)cc1]

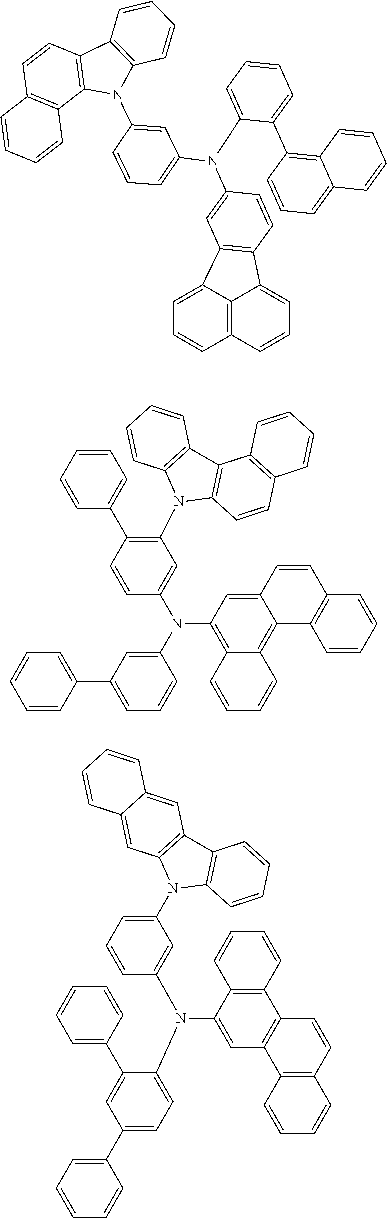

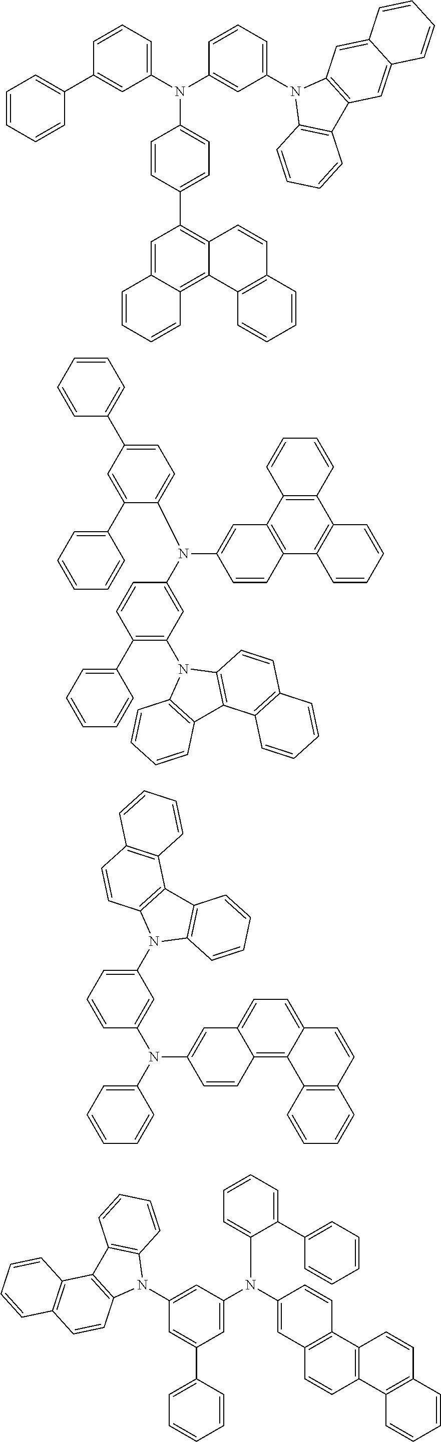

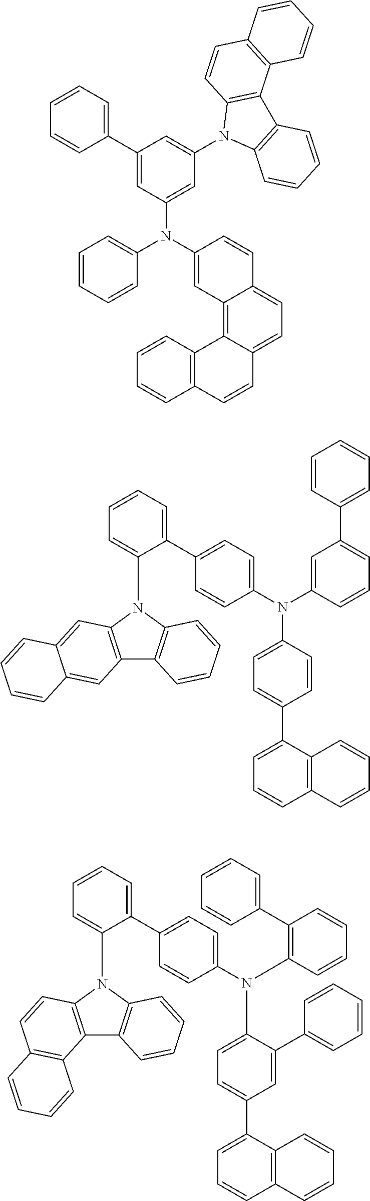 [SMILES: c1ccc(-c2cc(N(c3ccccc3)c3ccc4ccc5ccc6ccccc6c5c4c3)cc(-n3c4ccccc4c4c5ccccc5ccc43)c2)cc1 ; c1ccc(-c2cccc(N(c3ccc(-c4ccccc4-n4c5ccccc5c5cc6ccccc6cc54)cc3)c3ccc(-c4cccc5ccccc45)cc3)c2)cc1 ; c1ccc(-c2ccccc2N(c2ccc(-c3ccccc3-n3c4ccccc4c4c5ccccc5ccc43)cc2)c2ccc(-c3cccc4ccccc34)cc2-c2ccccc2)cc1]

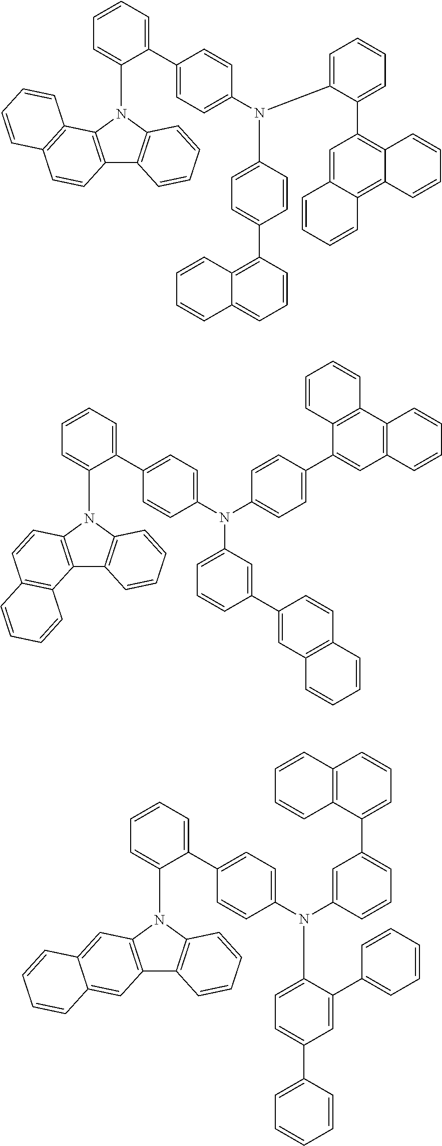 [SMILES: c1ccc(N(c2ccc(-c3ccccc3-n3c4ccccc4c4ccc5ccccc5c43)cc2)c2ccc(-c3cccc4ccccc34)cc2)c(-c2cc3ccccc3c3ccccc23)c1 ; c1cc(-c2ccc3ccccc3c2)cc(N(c2ccc(-c3ccccc3-n3c4ccccc4c4c5ccccc5ccc43)cc2)c2ccc(-c3cc4ccccc4c4ccccc34)cc2)c1 ; c1ccc(-c2ccc(N(c3ccc(-c4ccccc4-n4c5ccccc5c5cc6ccccc6cc54)cc3)c3cccc(-c4cccc5ccccc45)c3)c(-c3ccccc3)c2)cc1]

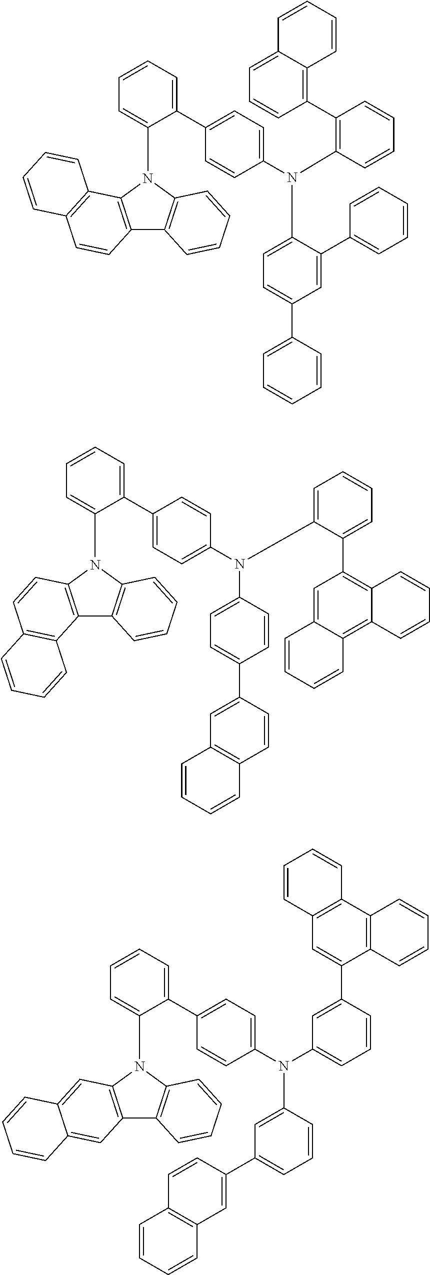 [SMILES: c1ccc(-c2ccc(N(c3ccc(-c4ccccc4-n4c5ccccc5c5ccc6ccccc6c54)cc3)c3ccccc3-c3cccc4ccccc34)c(-c3ccccc3)c2)cc1 ; c1ccc(N(c2ccc(-c3ccc4ccccc4c3)cc2)c2ccc(-c3ccccc3-n3c4ccccc4c4c5ccccc5ccc43)cc2)c(-c2cc3ccccc3c3ccccc23)c1 ; c1cc(-c2ccc3ccccc3c2)cc(N(c2ccc(-c3ccccc3-n3c4ccccc4c4cc5ccccc5cc43)cc2)c2cccc(-c3cc4ccccc4c4ccccc34)c2)c1]

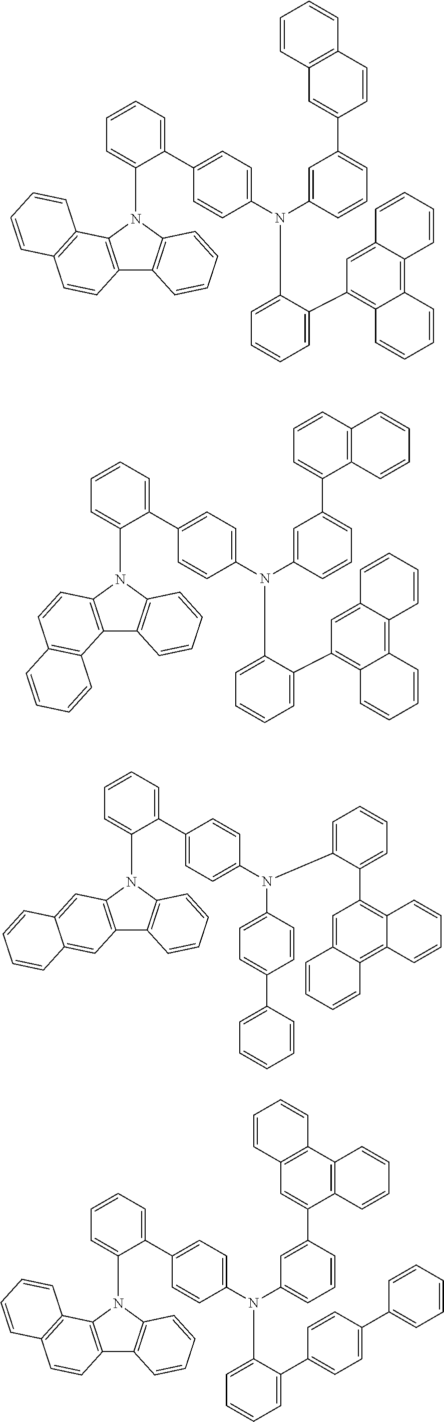

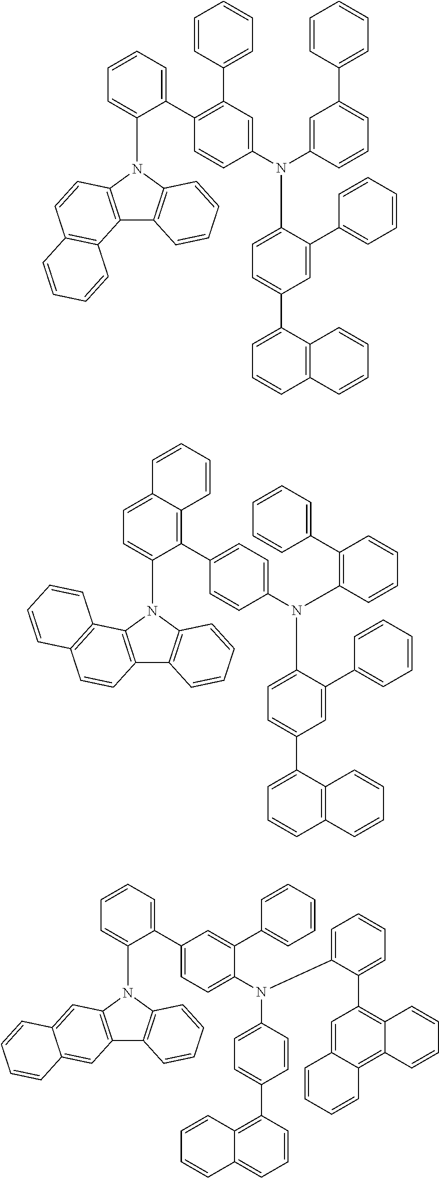 [SMILES: c1ccc(-c2cccc(N(c3ccc(-c4ccccc4-n4c5ccccc5c5c6ccccc6ccc54)c(-c4ccccc4)c3)c3ccc(-c4cccc5ccccc45)cc3-c3ccccc3)c2)cc1 ; c1ccc(-c2ccccc2N(c2ccc(-c3c(-n4c5ccccc5c5ccc6ccccc6c54)ccc4ccccc34)cc2)c2ccc(-c3cccc4ccccc34)cc2-c2ccccc2)cc1 ; c1ccc(-c2cc(-c3ccccc3-n3c4ccccc4c4cc5ccccc5cc43)ccc2N(c2ccc(-c3cccc4ccccc34)cc2)c2ccccc2-c2cc3ccccc3c3ccccc23)cc1]

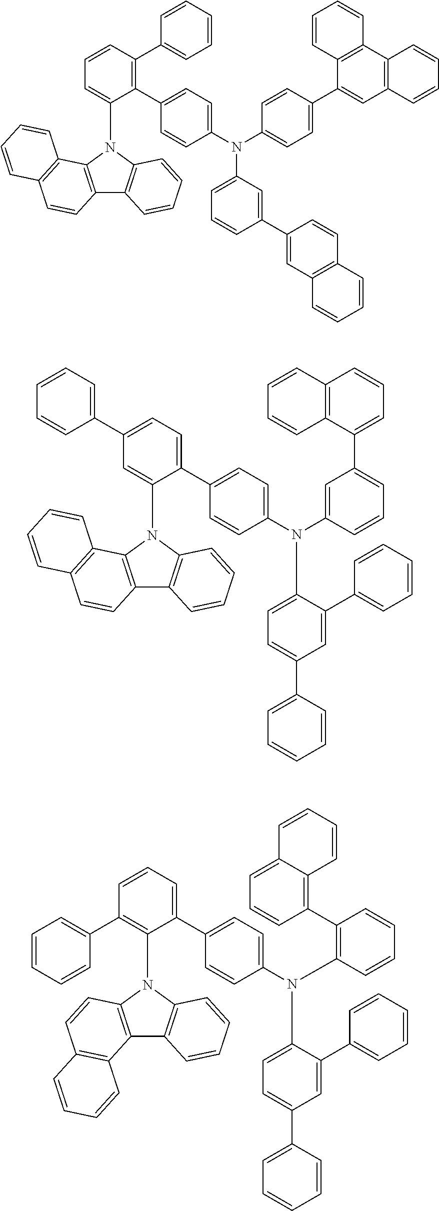 [SMILES: c1ccc(-c2cccc(-n3c4ccccc4c4ccc5ccccc5c43)c2-c2ccc(N(c3ccc(-c4cc5ccccc5c5ccccc45)cc3)c3cccc(-c4ccc5ccccc5c4)c3)cc2)cc1 ; c1ccc(-c2ccc(N(c3ccc(-c4ccc(-c5ccccc5)cc4-n4c5ccccc5c5ccc6ccccc6c54)cc3)c3cccc(-c4cccc5ccccc45)c3)c(-c3ccccc3)c2)cc1 ; c1ccc(-c2ccc(N(c3ccc(-c4cccc(-c5ccccc5)c4-n4c5ccccc5c5c6ccccc6ccc54)cc3)c3ccccc3-c3cccc4ccccc34)c(-c3ccccc3)c2)cc1]

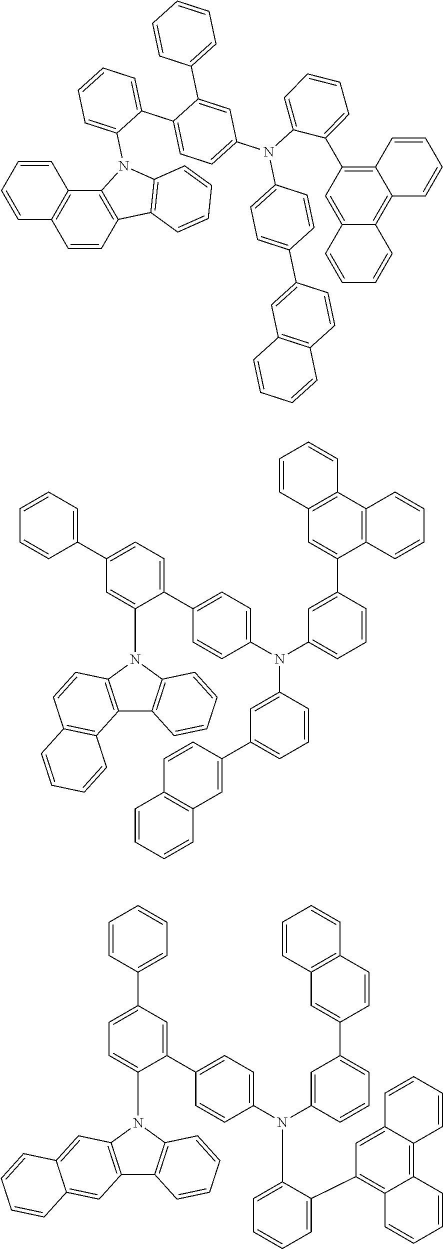

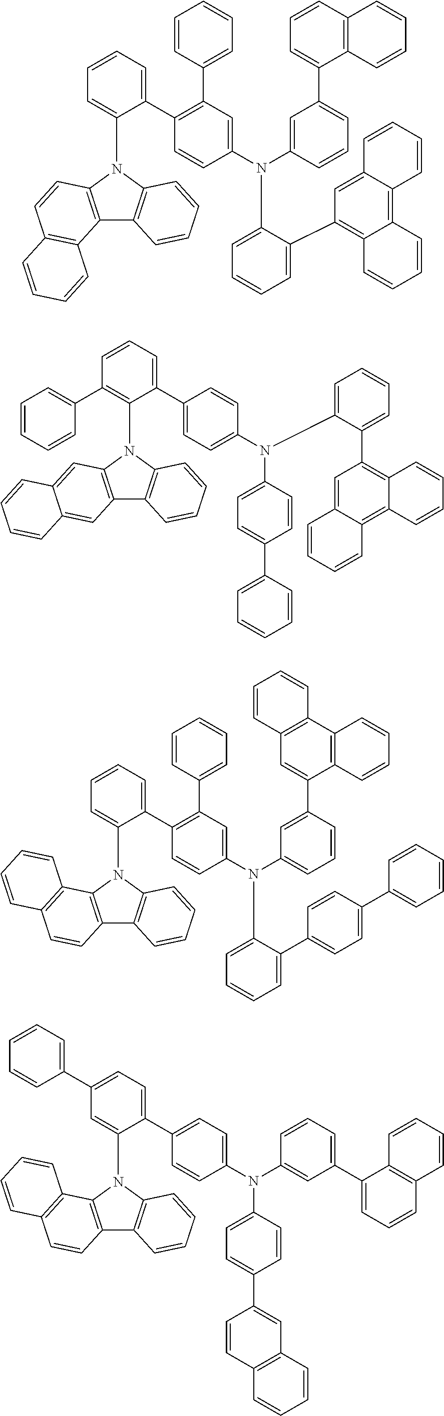

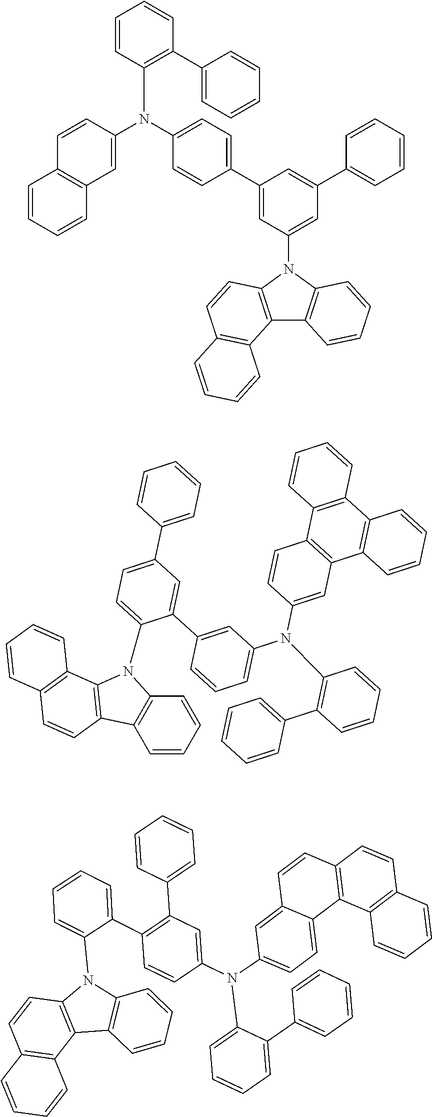 [SMILES: c1ccc(-c2cc(-c3ccc(N(c4ccc5ccccc5c4)c4ccccc4-c4ccccc4)cc3)cc(-n3c4ccccc4c4c5ccccc5ccc43)c2)cc1 ; c1ccc(-c2ccc(-n3c4ccccc4c4ccc5ccccc5c43)c(-c3cccc(N(c4ccc5c6ccccc6c6ccccc6c5c4)c4ccccc4-c4ccccc4)c3)c2)cc1 ; c1ccc(-c2cc(N(c3ccc4c(ccc5ccc6ccccc6c54)c3)c3ccccc3-c3ccccc3)ccc2-c2ccccc2-n2c3ccccc3c3c4ccccc4ccc32)cc1]

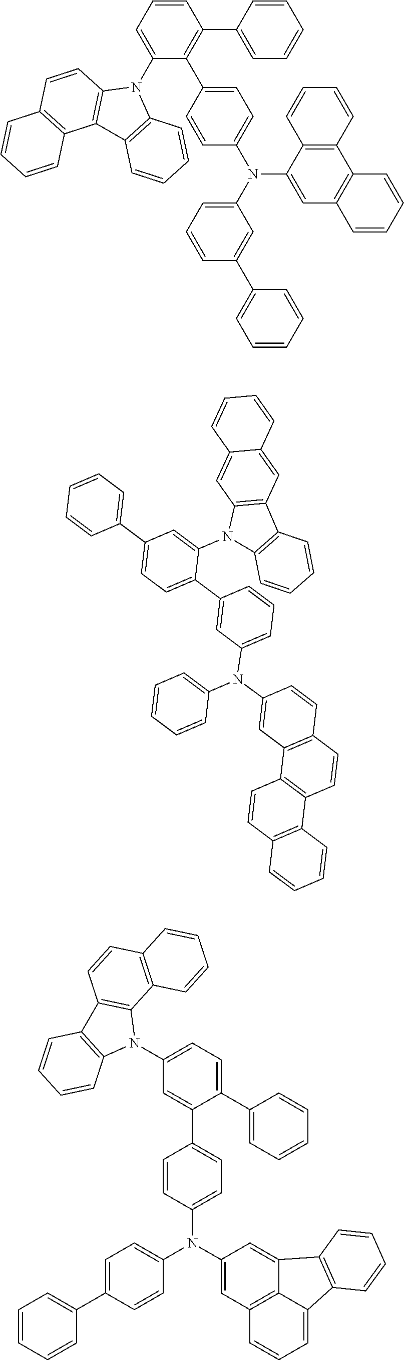 [SMILES: c1ccc(-c2cccc(N(c3ccc(-c4c(-c5ccccc5)cccc4-n4c5ccccc5c5c6ccccc6ccc54)cc3)c3cc4ccccc4c4ccccc34)c2)cc1 ; c1ccc(-c2ccc(-c3cccc(N(c4ccccc4)c4ccc5ccc6c7ccccc7ccc6c5c4)c3)c(-n3c4ccccc4c4cc5ccccc5cc43)c2)cc1 ; c1ccc(-c2ccc(N(c3ccc(-c4cc(-n5c6ccccc6c6ccc7ccccc7c65)ccc4-c4ccccc4)cc3)c3cc4c5c(cccc5c3)-c3ccccc3-4)cc2)cc1]

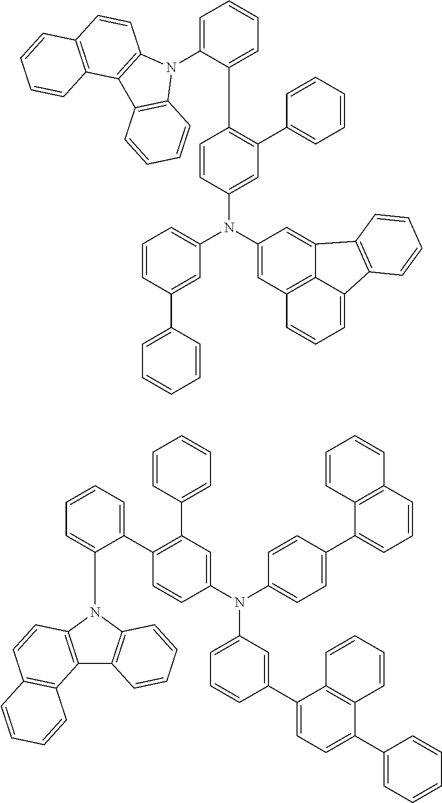

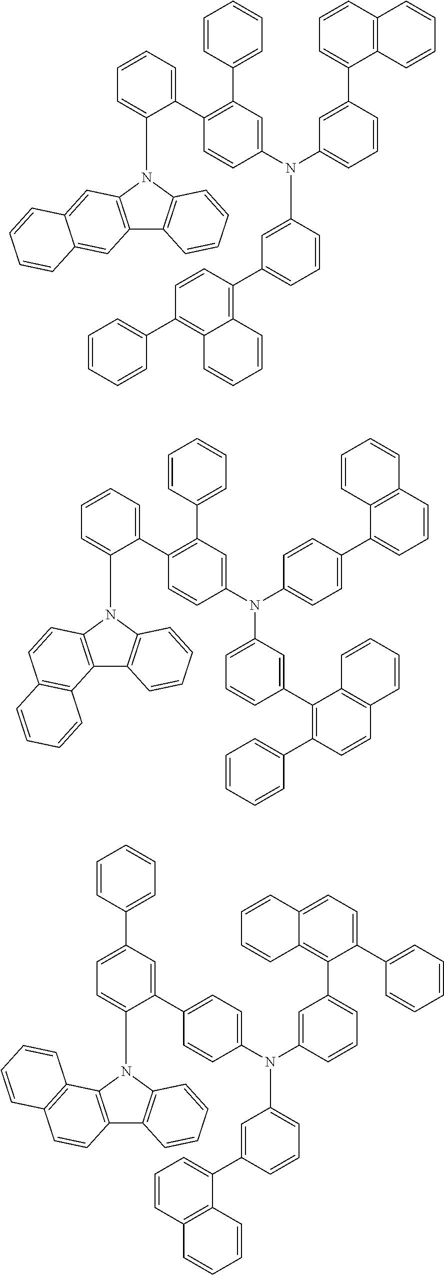 [SMILES: c1ccc(-c2cc(N(c3cccc(-c4cccc5ccccc45)c3)c3cccc(-c4ccc(-c5ccccc5)c5ccccc45)c3)ccc2-c2ccccc2-n2c3ccccc3c3cc4ccccc4cc32)cc1 ; c1ccc(-c2cc(N(c3ccc(-c4cccc5ccccc45)cc3)c3cccc(-c4c(-c5ccccc5)ccc5ccccc45)c3)ccc2-c2ccccc2-n2c3ccccc3c3c4ccccc4ccc32)cc1 ; c1ccc(-c2ccc(-n3c4ccccc4c4ccc5ccccc5c43)c(-c3ccc(N(c4cccc(-c5cccc6ccccc56)c4)c4cccc(-c5c(-c6ccccc6)ccc6ccccc56)c4)cc3)c2)cc1]

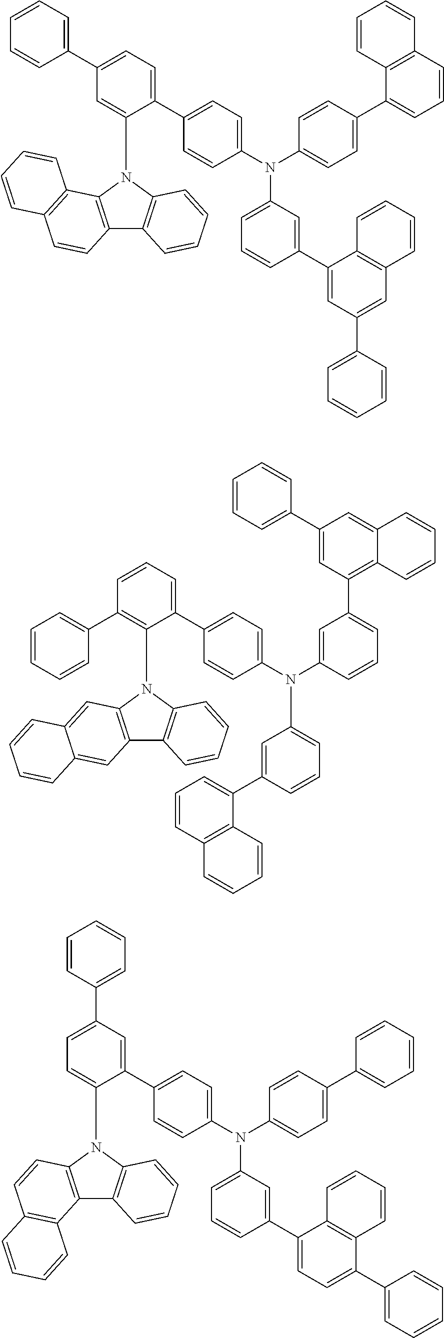 [SMILES: c1ccc(-c2ccc(-c3ccc(N(c4ccc(-c5cccc6ccccc56)cc4)c4cccc(-c5cc(-c6ccccc6)cc6ccccc56)c4)cc3)c(-n3c4ccccc4c4ccc5ccccc5c43)c2)cc1 ; c1ccc(-c2cc(-c3cccc(N(c4ccc(-c5cccc(-c6ccccc6)c5-n5c6ccccc6c6cc7ccccc7cc65)cc4)c4cccc(-c5cccc6ccccc56)c4)c3)c3ccccc3c2)cc1 ; c1ccc(-c2ccc(N(c3ccc(-c4cc(-c5ccccc5)ccc4-n4c5ccccc5c5c6ccccc6ccc54)cc3)c3cccc(-c4ccc(-c5ccccc5)c5ccccc45)c3)cc2)cc1]

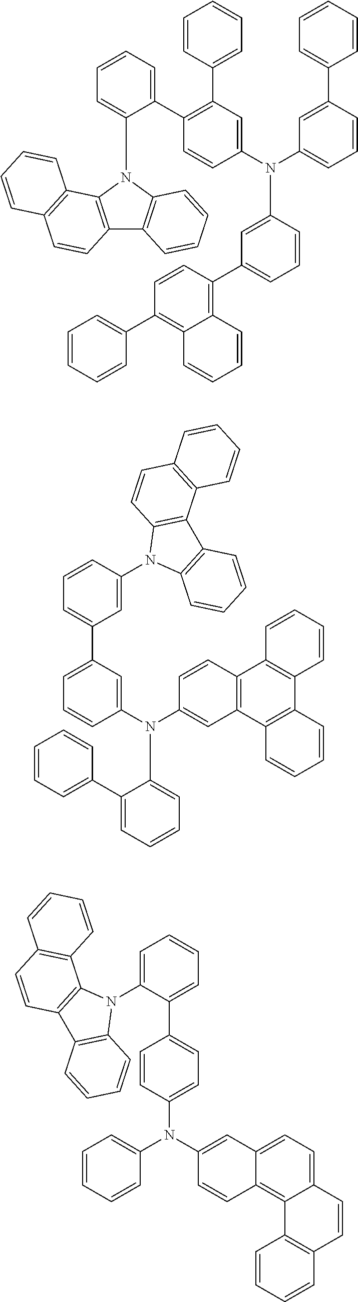

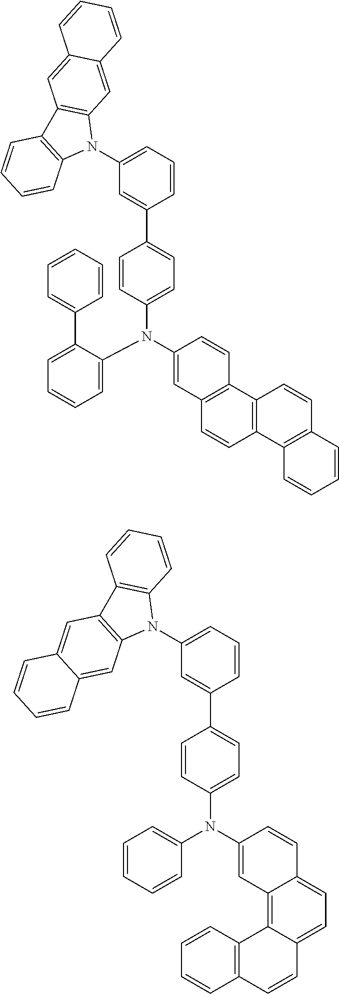 [SMILES: c1ccc(-c2ccccc2N(c2ccc(-c3cccc(-n4c5ccccc5c5cc6ccccc6cc54)c3)cc2)c2ccc3c(ccc4c5ccccc5ccc34)c2)cc1 ; c1ccc(N(c2ccc(-c3cccc(-n4c5ccccc5c5cc6ccccc6cc54)c3)cc2)c2ccc3ccc4ccc5ccccc5c4c3c2)cc1]

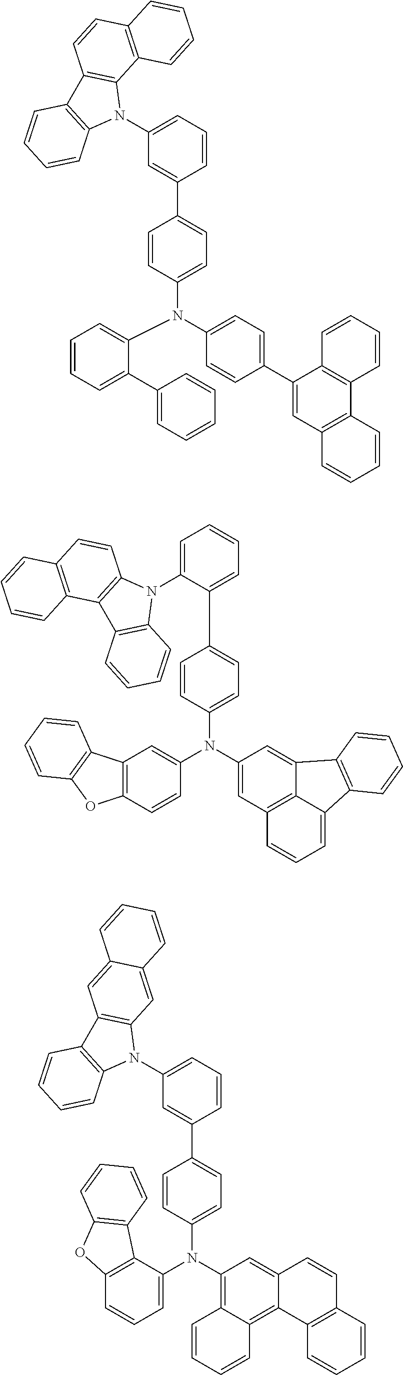 [SMILES: c1ccc(-c2ccccc2N(c2ccc(-c3cccc(-n4c5ccccc5c5ccc6ccccc6c54)c3)cc2)c2ccc(-c3cc4ccccc4c4ccccc34)cc2)cc1 ; c1ccc2c(c1)-c1cccc3cc(N(c4ccc(-c5ccccc5-n5c6ccccc6c6c7ccccc7ccc65)cc4)c4ccc5oc6ccccc6c5c4)cc-2c13 ; c1cc(-c2ccc(N(c3cc4ccc5ccccc5c4c4ccccc34)c3cccc4oc5ccccc5c34)cc2)cc(-n2c3ccccc3c3cc4ccccc4cc32)c1]

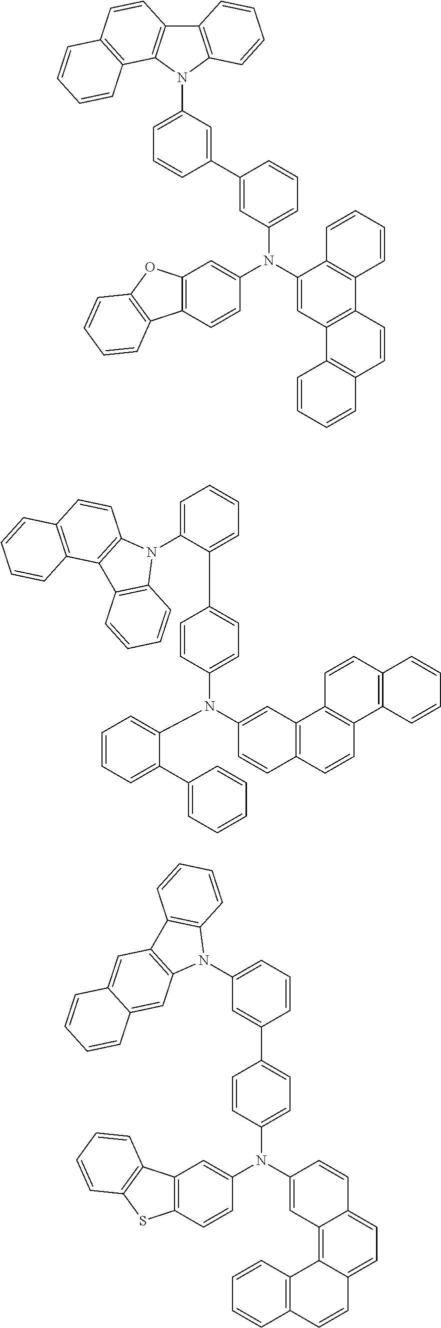

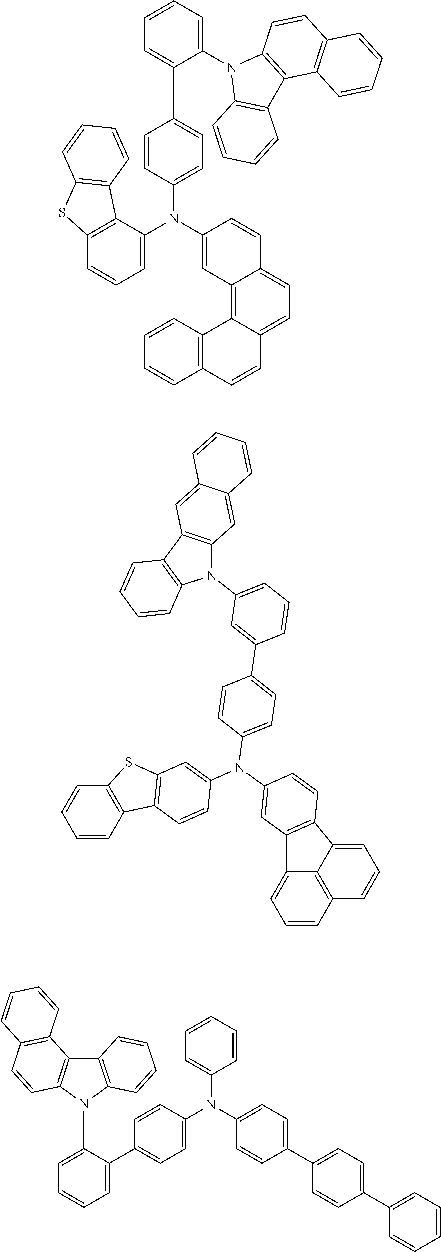 [SMILES: c1ccc(-n2c3ccccc3c3c4ccccc4ccc32)c(-c2ccc(N(c3ccc4ccc5ccc6ccccc6c5c4c3)c3cccc4sc5ccccc5c34)cc2)c1 ; c1cc(-c2ccc(N(c3ccc4c(c3)-c3cccc5cccc-4c35)c3ccc4c(c3)sc3ccccc34)cc2)cc(-n2c3ccccc3c3cc4ccccc4cc32)c1 ; c1ccc(-c2ccc(-c3ccc(N(c4ccccc4)c4ccc(-c5ccccc5-n5c6ccccc6c6c7ccccc7ccc65)cc4)cc3)cc2)cc1]

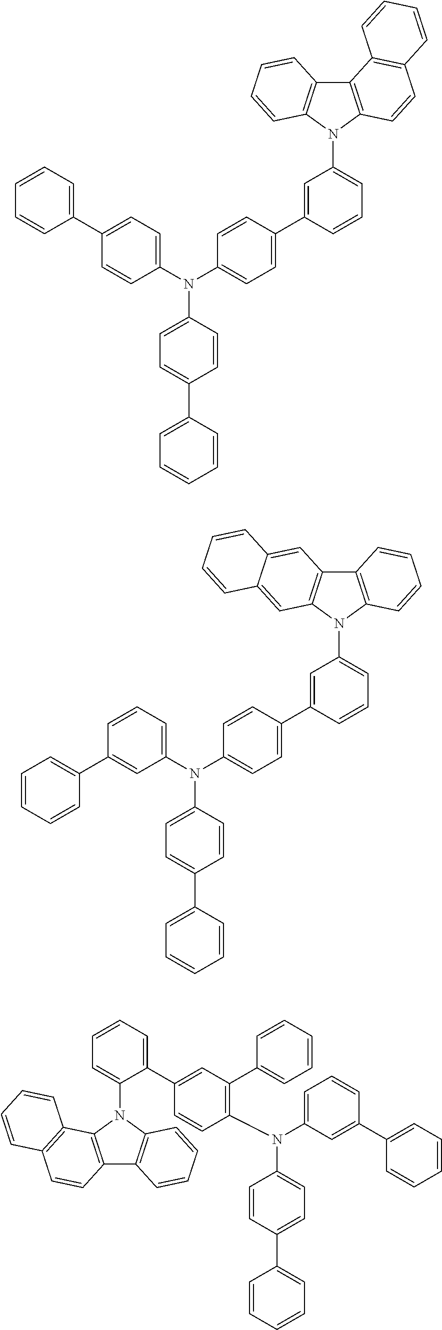 [SMILES: c1ccc(-c2ccc(N(c3ccc(-c4ccccc4)cc3)c3ccc(-c4cccc(-n5c6ccccc6c6c7ccccc7ccc65)c4)cc3)cc2)cc1 ; c1ccc(-c2ccc(N(c3ccc(-c4cccc(-n5c6ccccc6c6cc7ccccc7cc65)c4)cc3)c3cccc(-c4ccccc4)c3)cc2)cc1 ; c1ccc(-c2ccc(N(c3cccc(-c4ccccc4)c3)c3ccc(-c4ccccc4-n4c5ccccc5c5ccc6ccccc6c54)cc3-c3ccccc3)cc2)cc1]

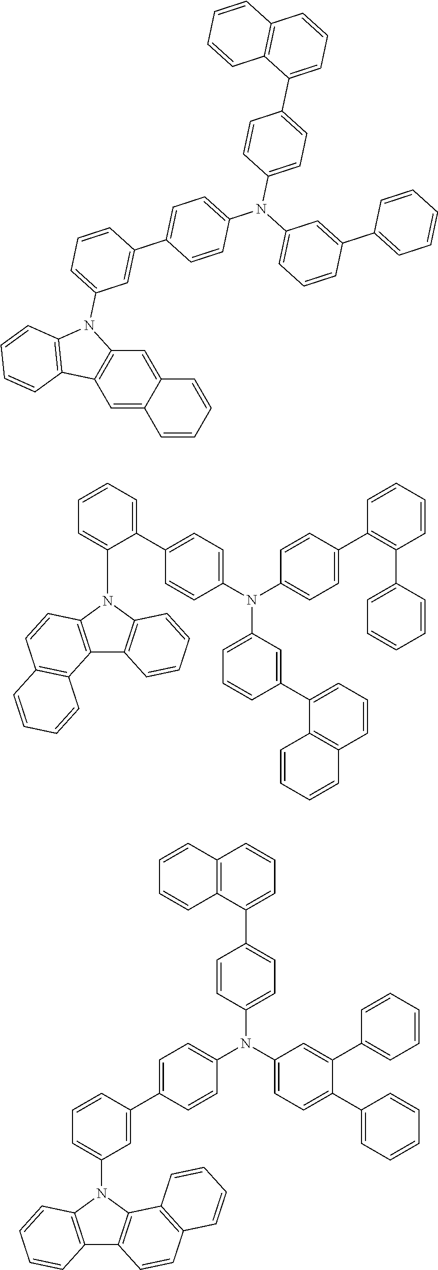 [SMILES: c1ccc(-c2cccc(N(c3ccc(-c4cccc(-n5c6ccccc6c6cc7ccccc7cc65)c4)cc3)c3ccc(-c4cccc5ccccc45)cc3)c2)cc1 ; c1ccc(-c2ccccc2-c2ccc(N(c3ccc(-c4ccccc4-n4c5ccccc5c5c6ccccc6ccc54)cc3)c3cccc(-c4cccc5ccccc45)c3)cc2)cc1 ; c1ccc(-c2ccc(N(c3ccc(-c4cccc(-n5c6ccccc6c6ccc7ccccc7c65)c4)cc3)c3ccc(-c4cccc5ccccc45)cc3)cc2-c2ccccc2)cc1]

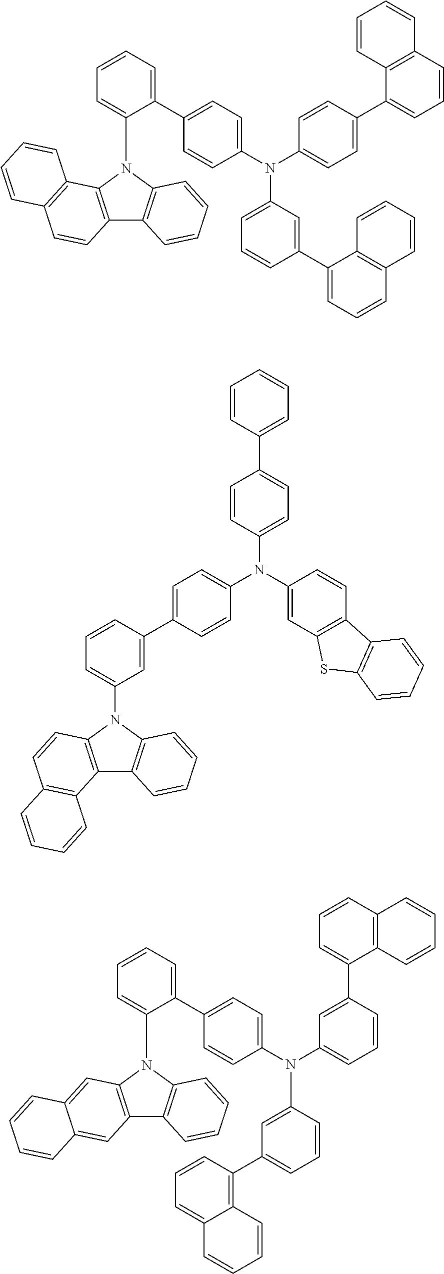 [SMILES: c1cc(-c2cccc3ccccc23)cc(N(c2ccc(-c3ccccc3-n3c4ccccc4c4ccc5ccccc5c43)cc2)c2ccc(-c3cccc4ccccc34)cc2)c1 ; c1ccc(-c2ccc(N(c3ccc(-c4cccc(-n5c6ccccc6c6c7ccccc7ccc65)c4)cc3)c3ccc4c(c3)sc3ccccc34)cc2)cc1 ; c1cc(-c2cccc3ccccc23)cc(N(c2ccc(-c3ccccc3-n3c4ccccc4c4cc5ccccc5cc43)cc2)c2cccc(-c3cccc4ccccc34)c2)c1]

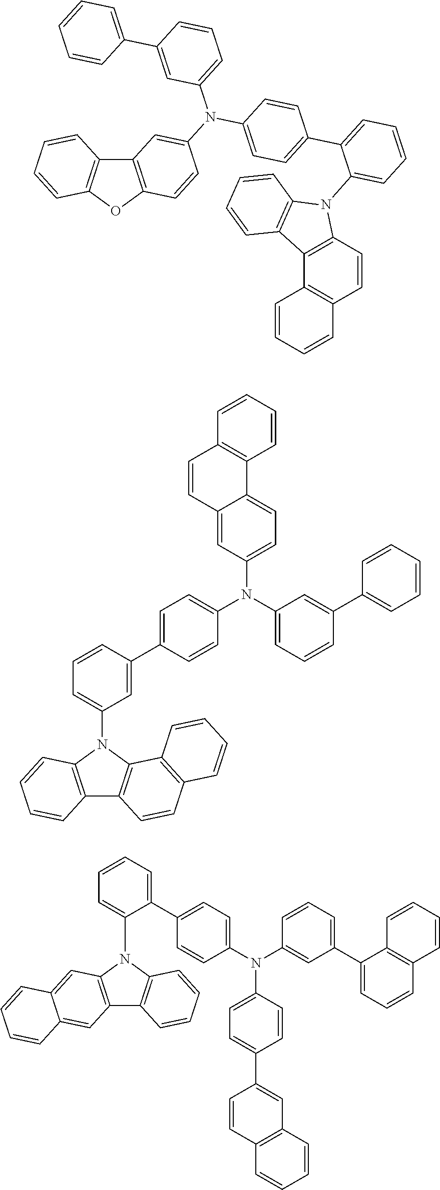

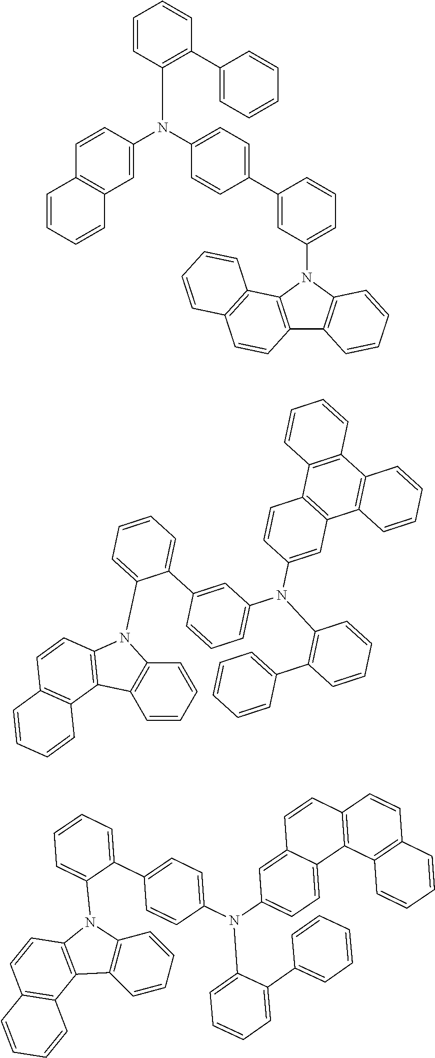 [SMILES: c1ccc(-c2ccccc2N(c2ccc(-c3cccc(-n4c5ccccc5c5ccc6ccccc6c54)c3)cc2)c2ccc3ccccc3c2)cc1 ; c1ccc(-c2ccccc2N(c2cccc(-c3ccccc3-n3c4ccccc4c4c5ccccc5ccc43)c2)c2ccc3c4ccccc4c4ccccc4c3c2)cc1 ; c1ccc(-c2ccccc2N(c2ccc(-c3ccccc3-n3c4ccccc4c4c5ccccc5ccc43)cc2)c2ccc3c(ccc4ccc5ccccc5c43)c2)cc1]

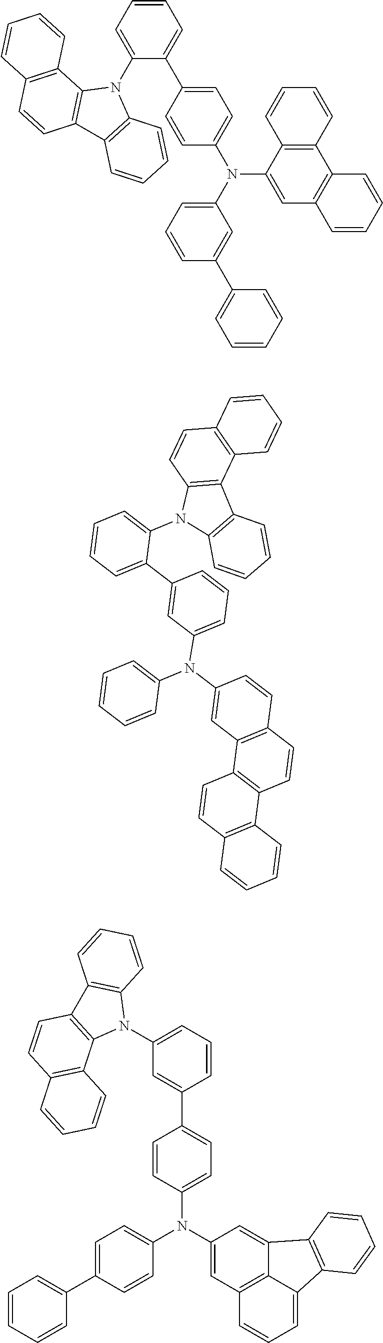 [SMILES: c1ccc(-c2cccc(N(c3ccc(-c4ccccc4-n4c5ccccc5c5ccc6ccccc6c54)cc3)c3cc4ccccc4c4ccccc34)c2)cc1 ; c1ccc(N(c2cccc(-c3ccccc3-n3c4ccccc4c4c5ccccc5ccc43)c2)c2ccc3ccc4c5ccccc5ccc4c3c2)cc1 ; c1ccc(-c2ccc(N(c3ccc(-c4cccc(-n5c6ccccc6c6ccc7ccccc7c65)c4)cc3)c3cc4c5c(cccc5c3)-c3ccccc3-4)cc2)cc1]

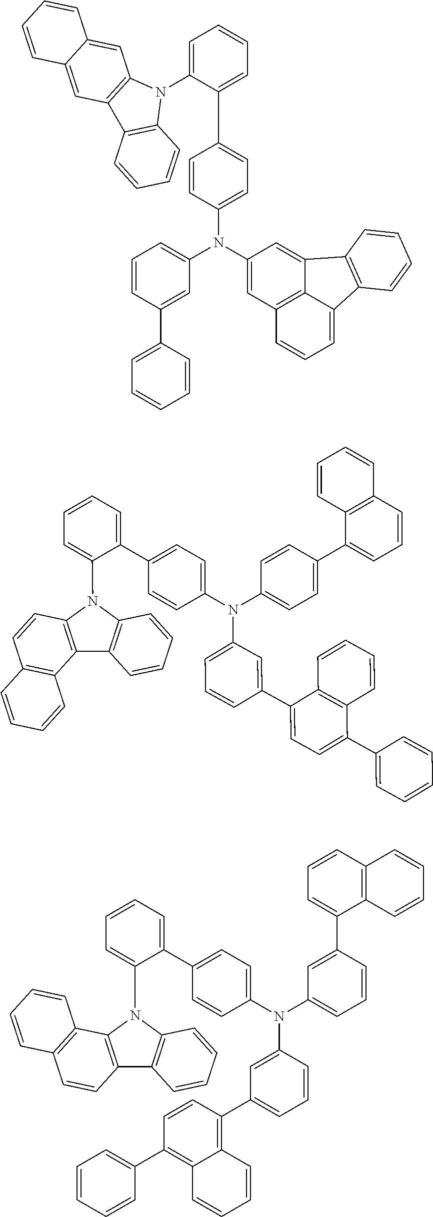 [SMILES: c1ccc(-c2cccc(N(c3ccc(-c4ccccc4-n4c5ccccc5c5cc6ccccc6cc54)cc3)c3cc4c5c(cccc5c3)-c3ccccc3-4)c2)cc1 ; c1ccc(-c2ccc(-c3cccc(N(c4ccc(-c5ccccc5-n5c6ccccc6c6c7ccccc7ccc65)cc4)c4ccc(-c5cccc6ccccc56)cc4)c3)c3ccccc23)cc1 ; c1ccc(-c2ccc(-c3cccc(N(c4ccc(-c5ccccc5-n5c6ccccc6c6ccc7ccccc7c65)cc4)c4cccc(-c5cccc6ccccc56)c4)c3)c3ccccc23)cc1]

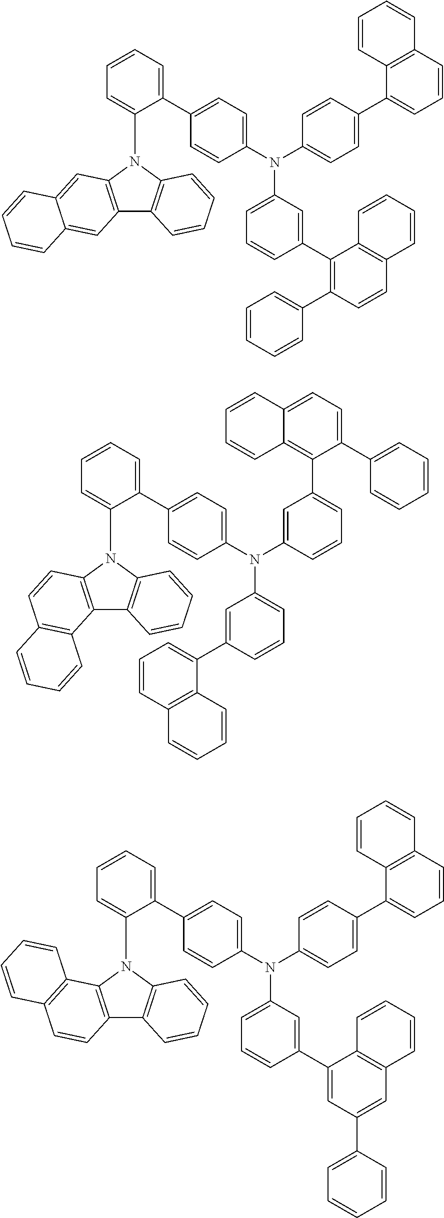

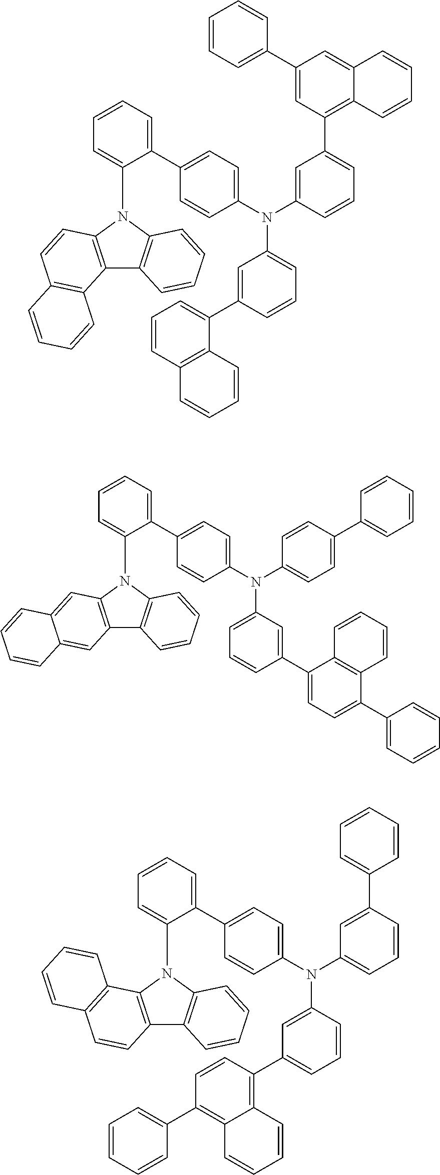 [SMILES: c1ccc(-c2cc(-c3cccc(N(c4ccc(-c5ccccc5-n5c6ccccc6c6c7ccccc7ccc65)cc4)c4cccc(-c5cccc6ccccc56)c4)c3)c3ccccc3c2)cc1 ; c1ccc(-c2ccc(N(c3ccc(-c4ccccc4-n4c5ccccc5c5cc6ccccc6cc54)cc3)c3cccc(-c4ccc(-c5ccccc5)c5ccccc45)c3)cc2)cc1 ; c1ccc(-c2cccc(N(c3ccc(-c4ccccc4-n4c5ccccc5c5ccc6ccccc6c54)cc3)c3cccc(-c4ccc(-c5ccccc5)c5ccccc45)c3)c2)cc1]

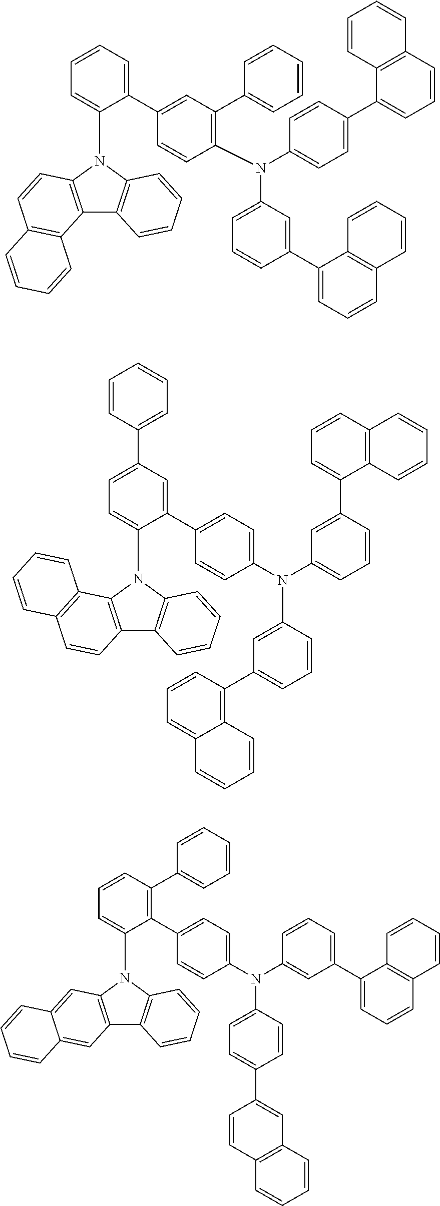

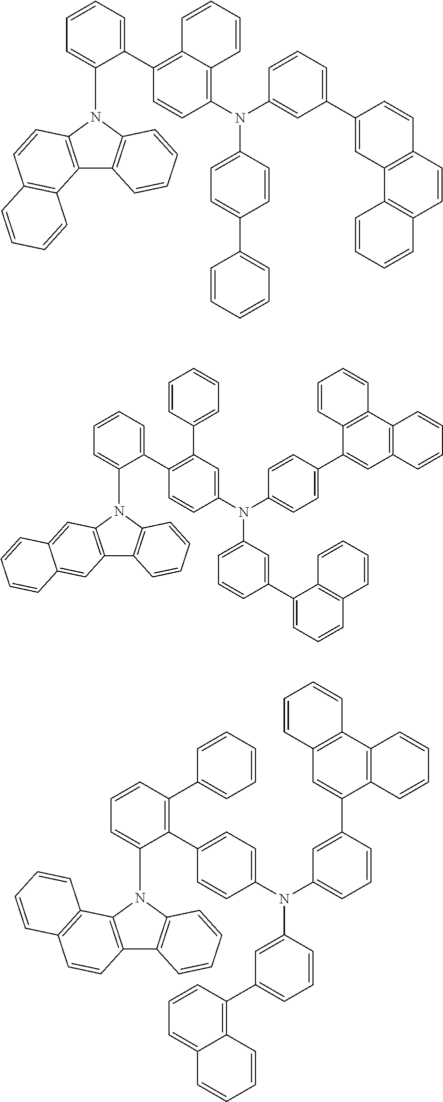

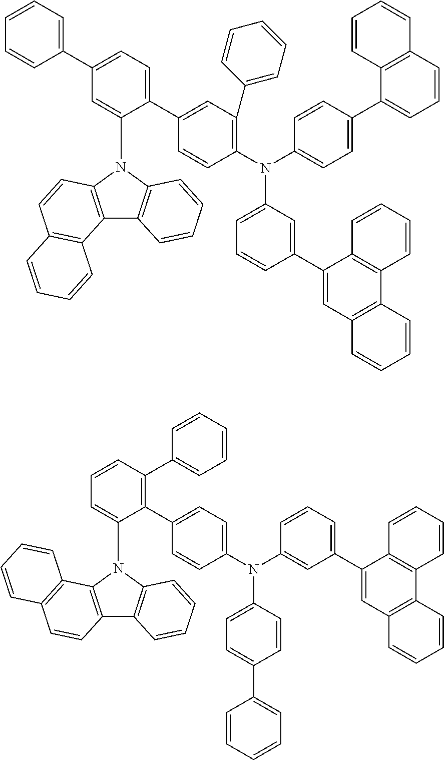 [SMILES: c1ccc(-c2ccc(-c3ccc(N(c4ccc(-c5cccc6ccccc56)cc4)c4cccc(-c5cc6ccccc6c6ccccc56)c4)c(-c4ccccc4)c3)c(-n3c4ccccc4c4c5ccccc5ccc43)c2)cc1 ; c1ccc(-c2ccc(N(c3ccc(-c4c(-c5ccccc5)cccc4-n4c5ccccc5c5ccc6ccccc6c54)cc3)c3cccc(-c4cc5ccccc5c5ccccc45)c3)cc2)cc1]

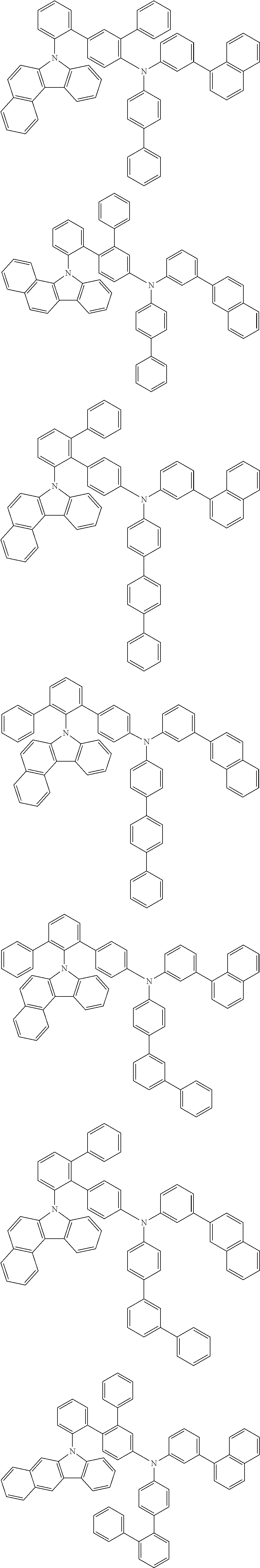

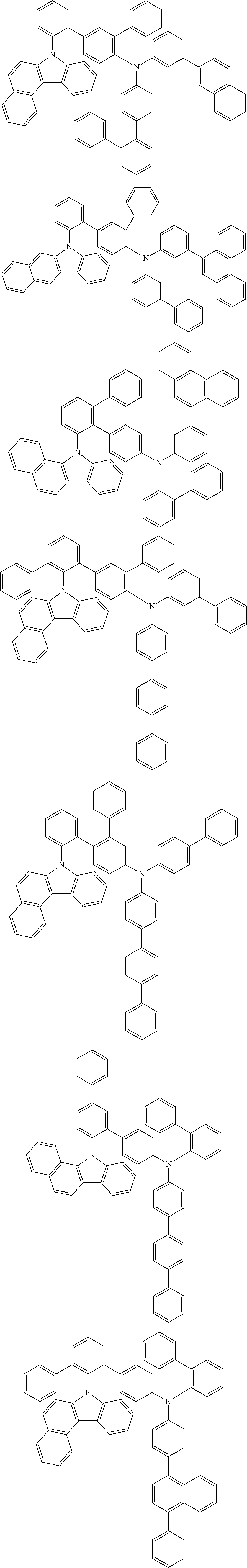

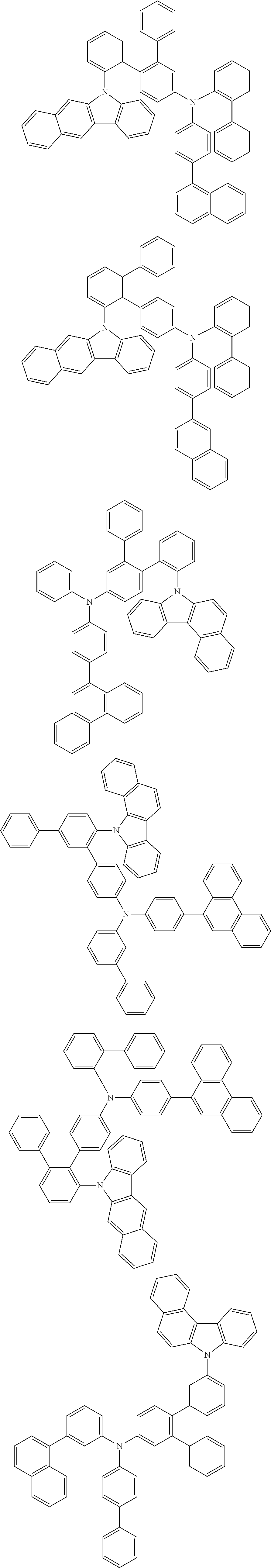

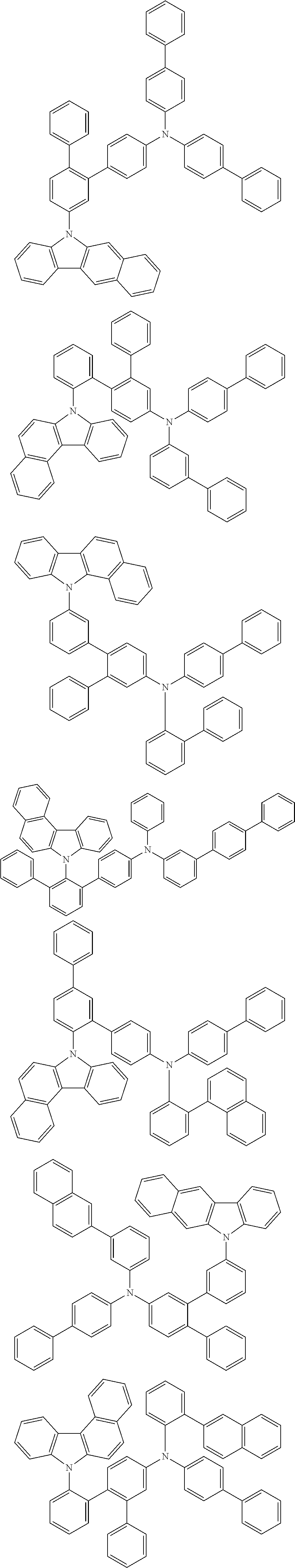

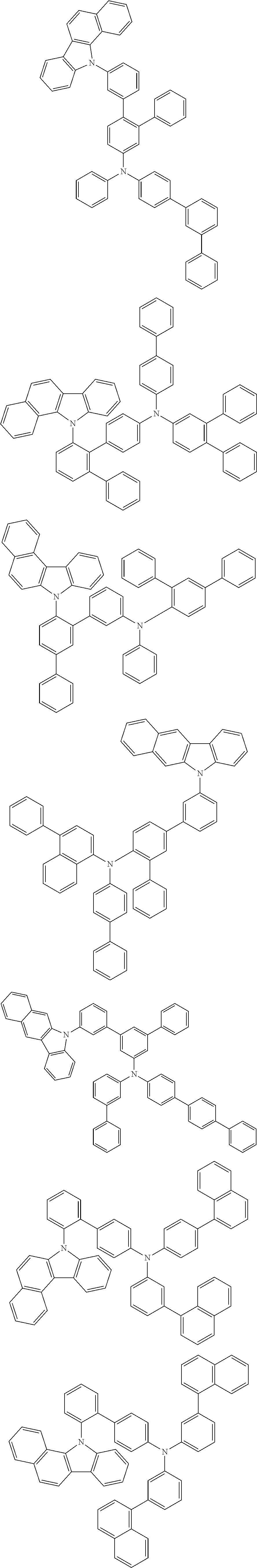

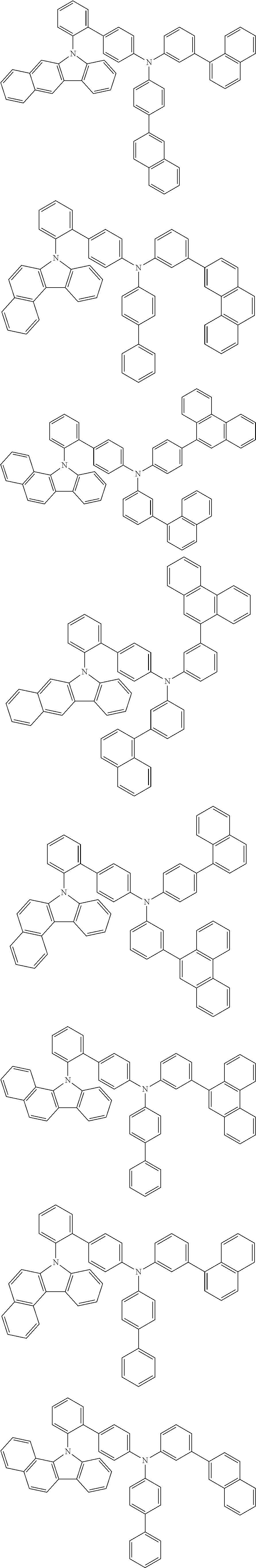

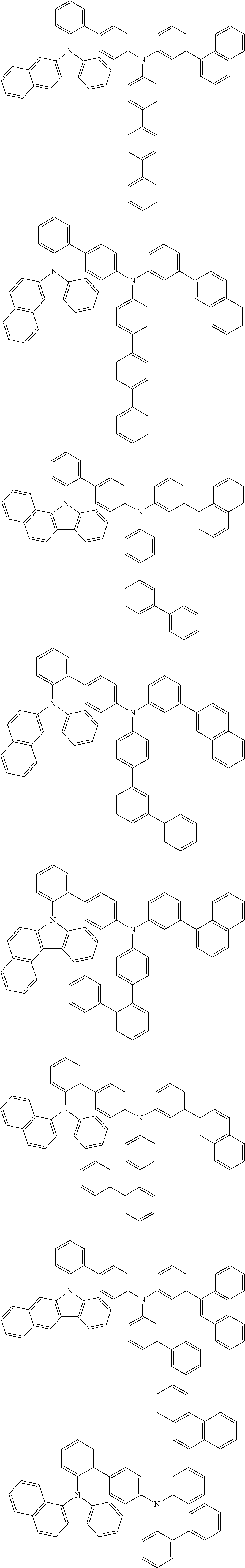

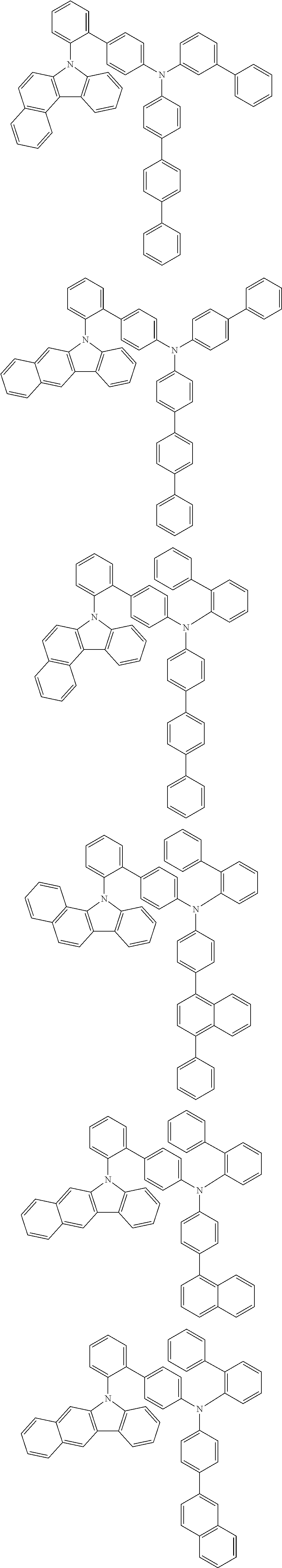

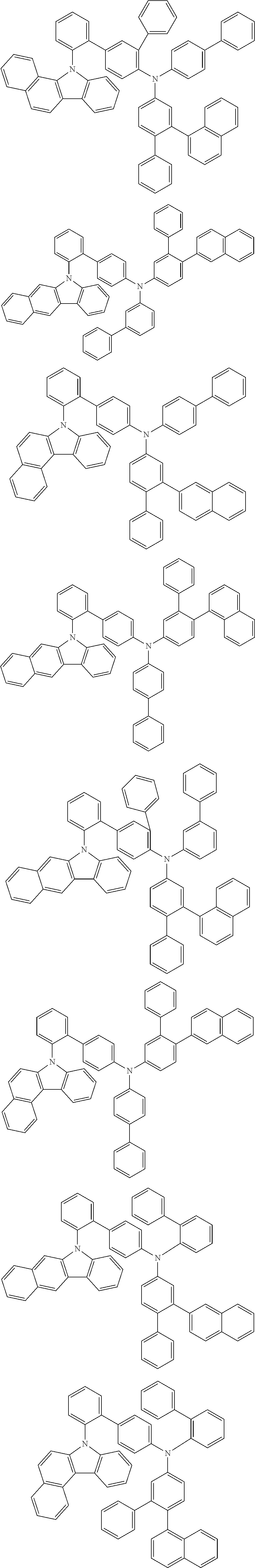

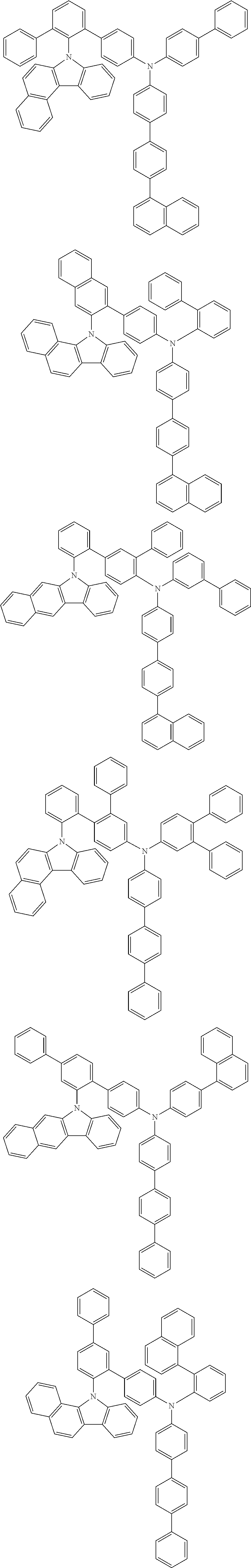

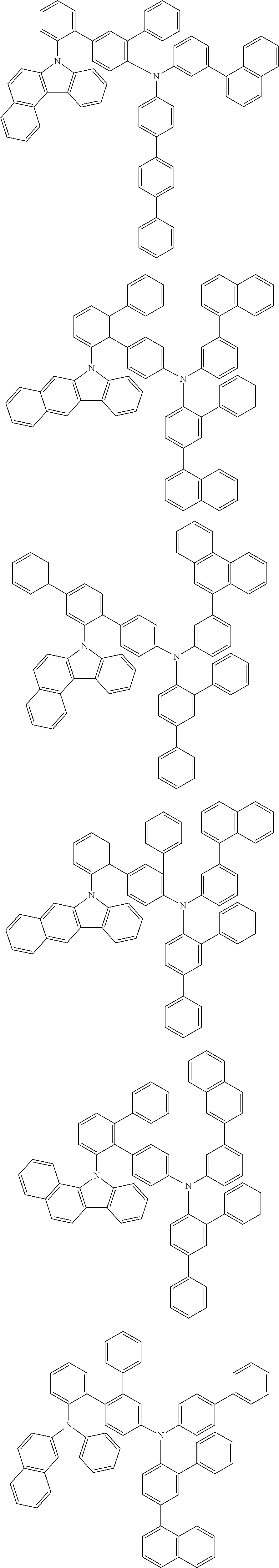

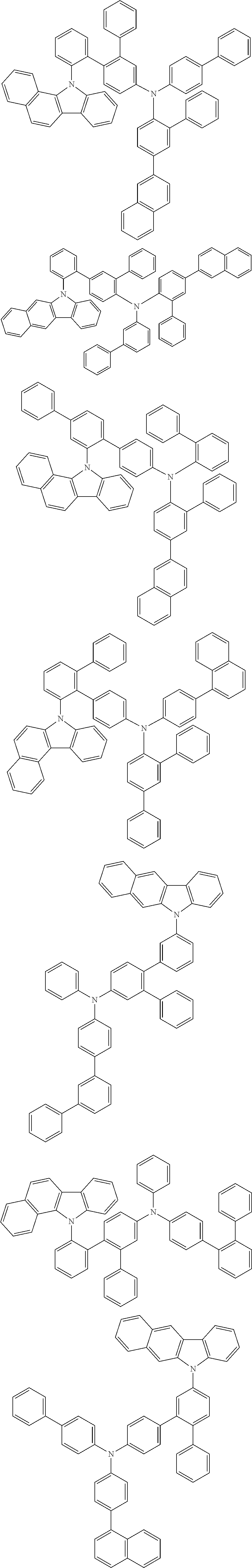

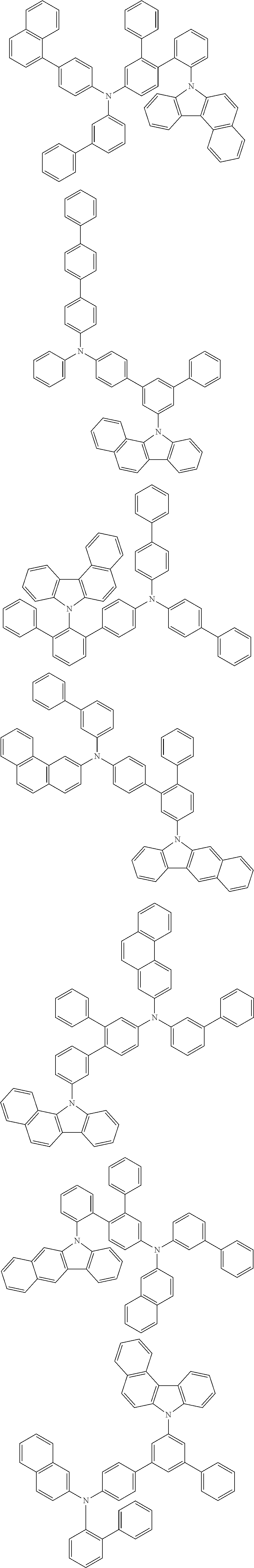

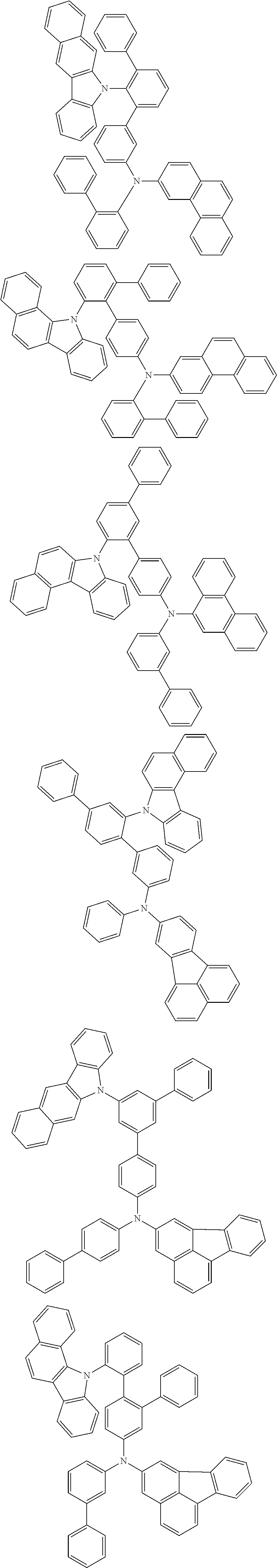

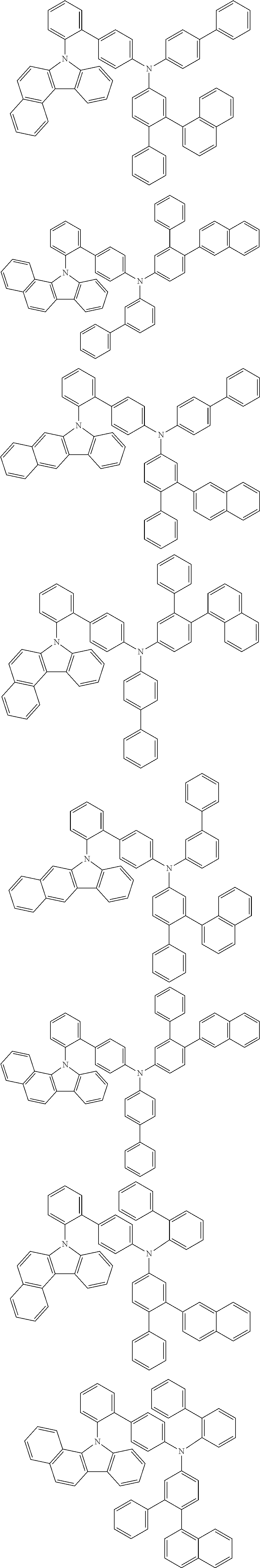

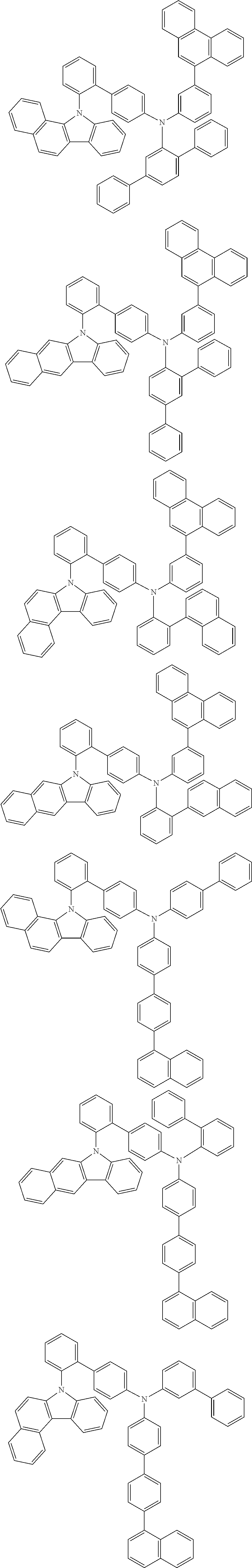

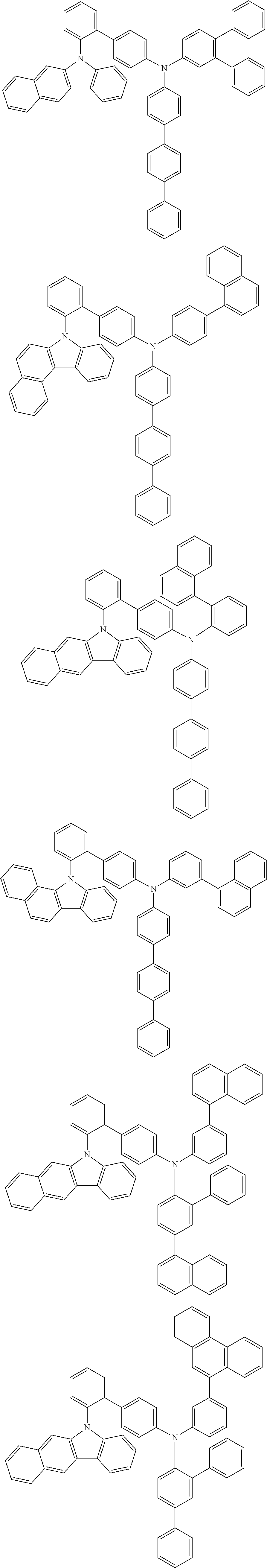

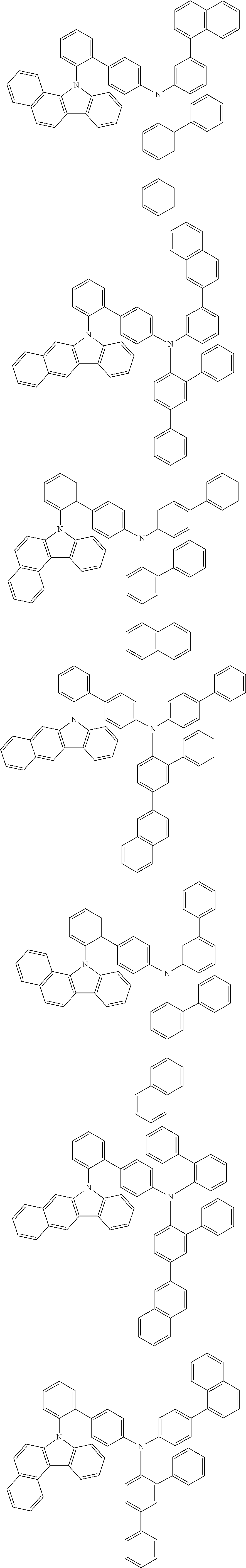

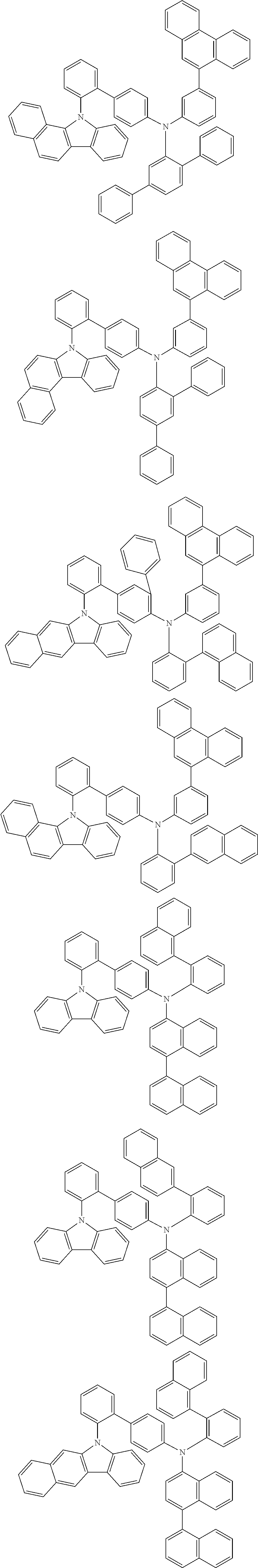

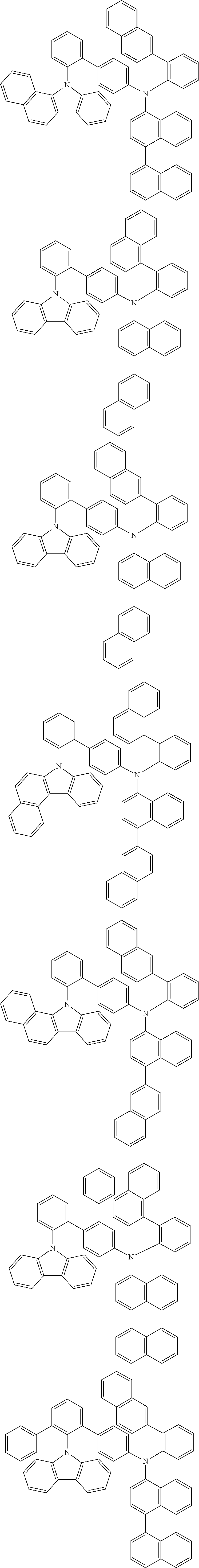

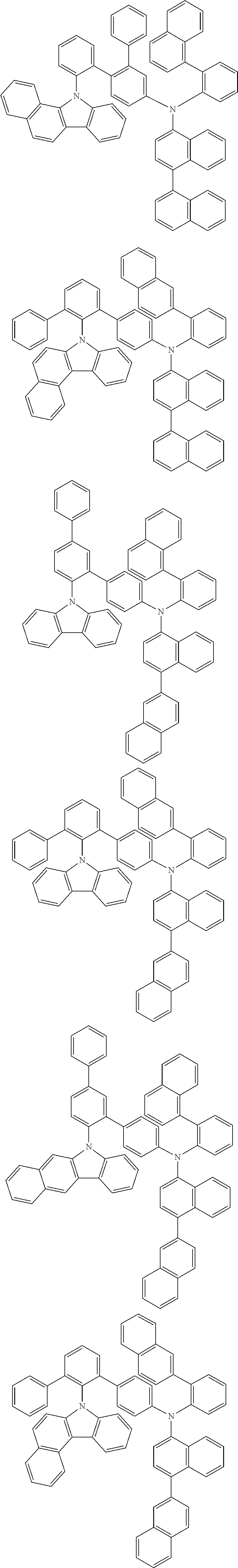

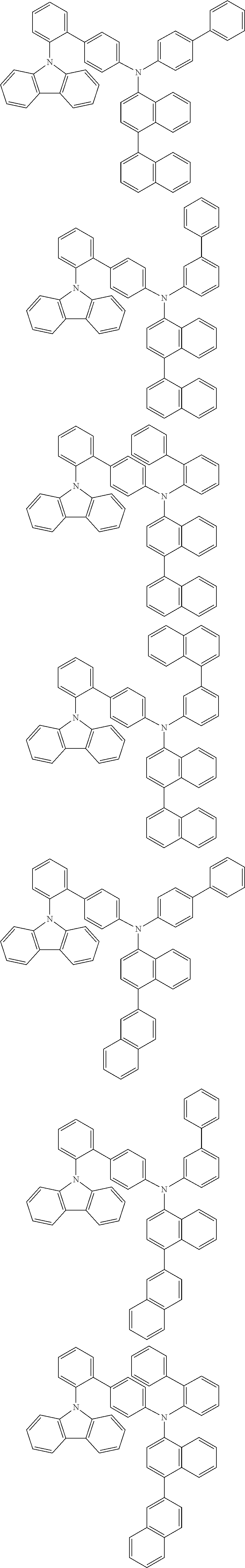

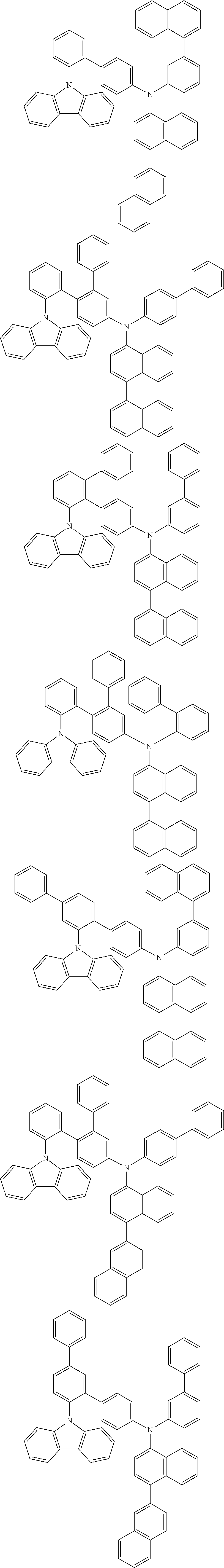

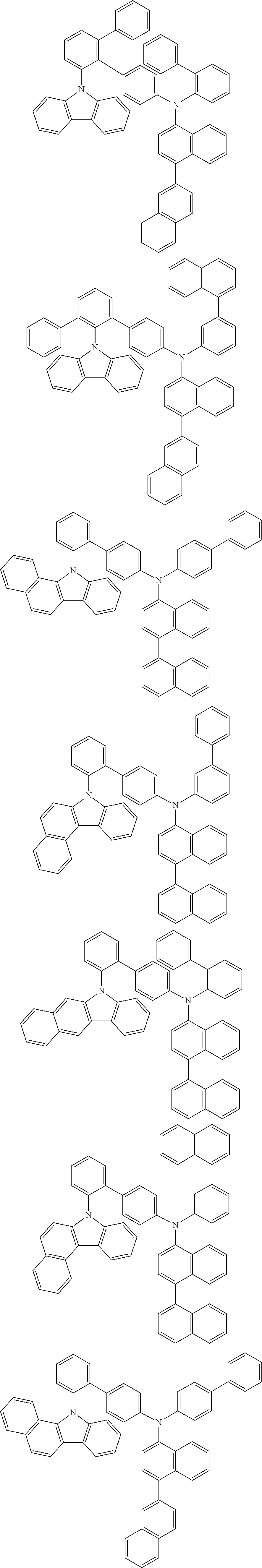

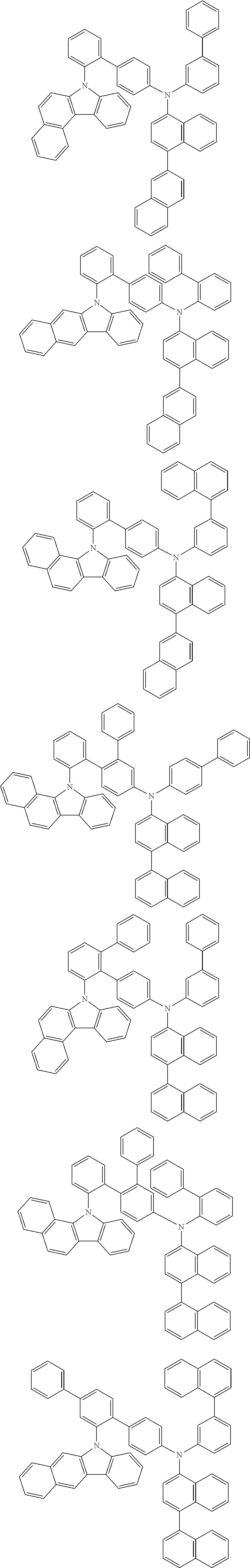

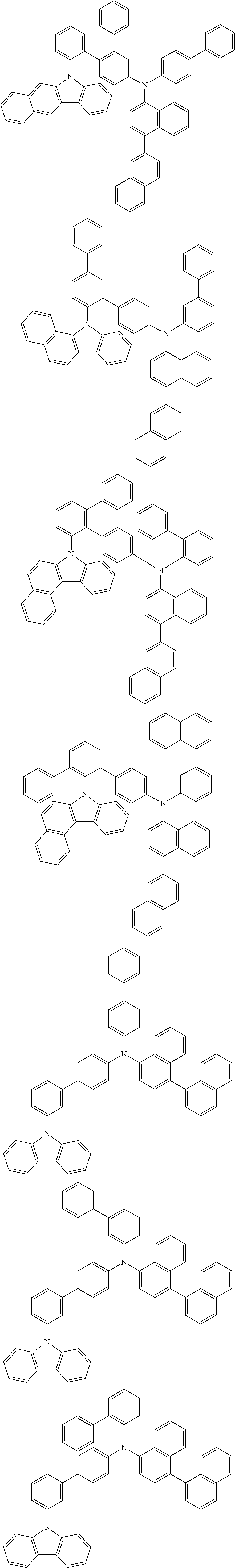

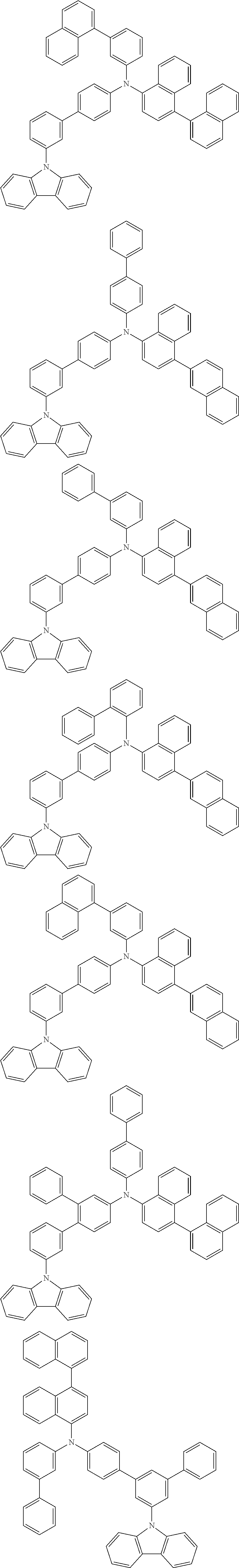

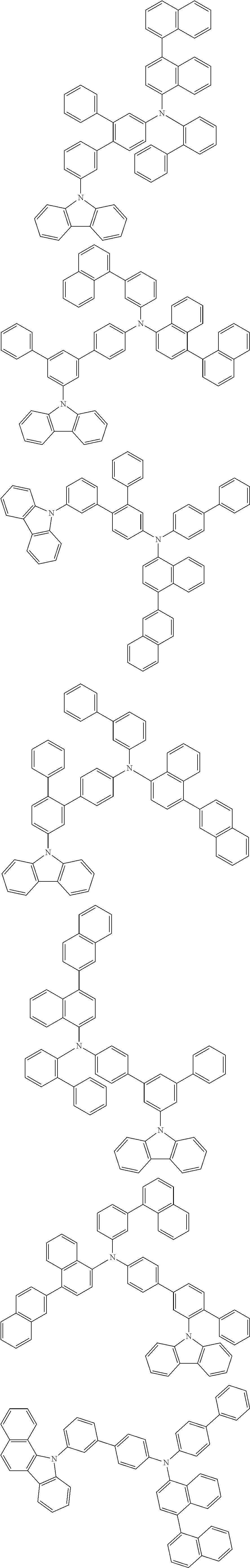

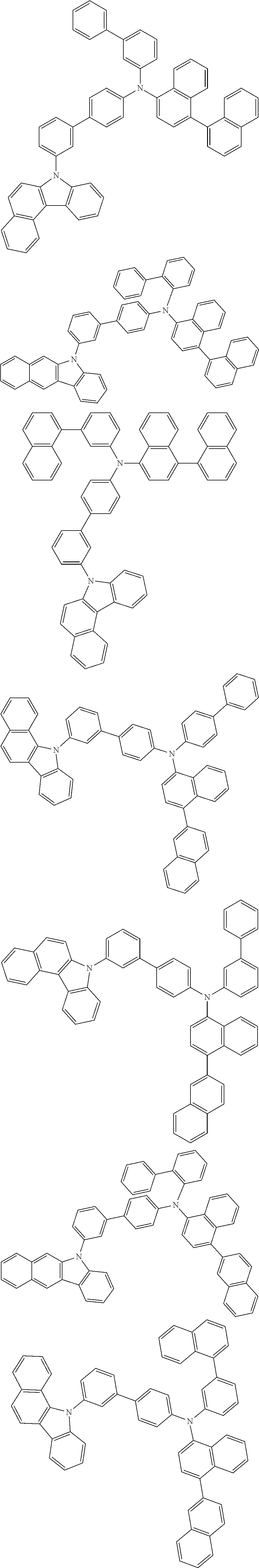

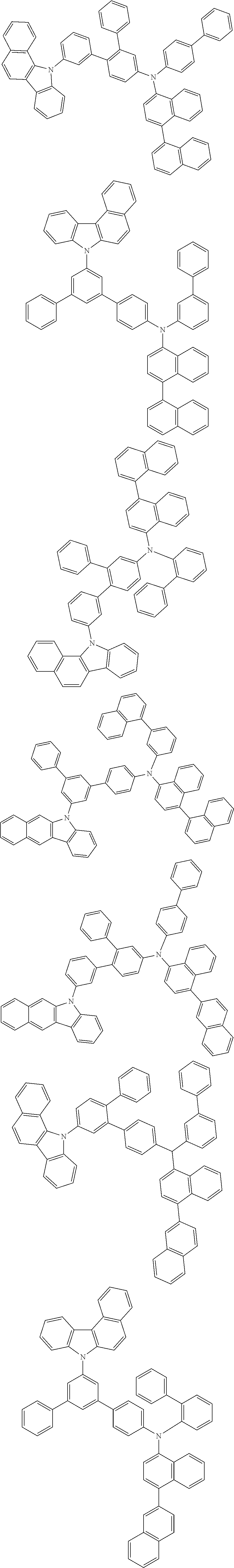

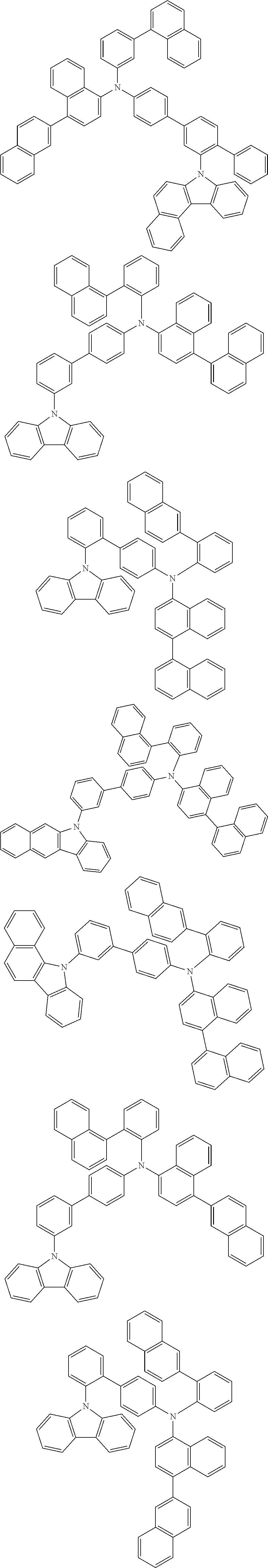

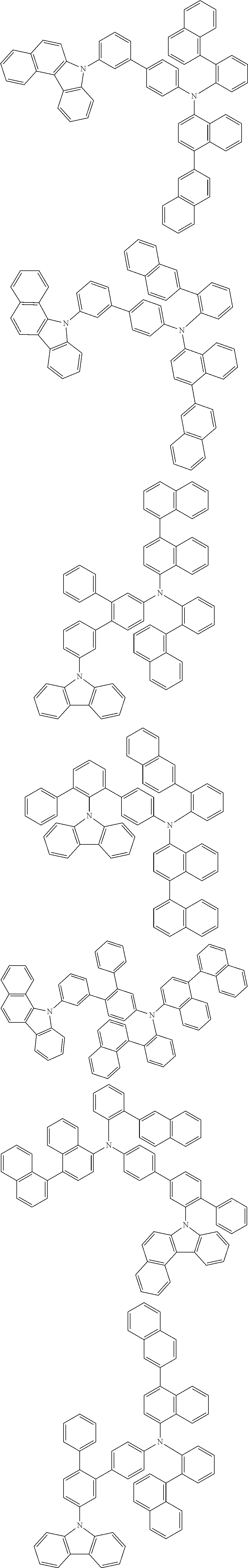

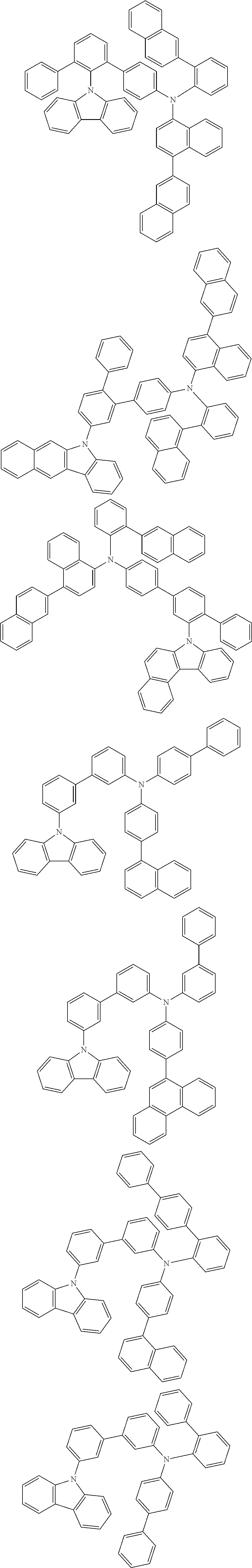

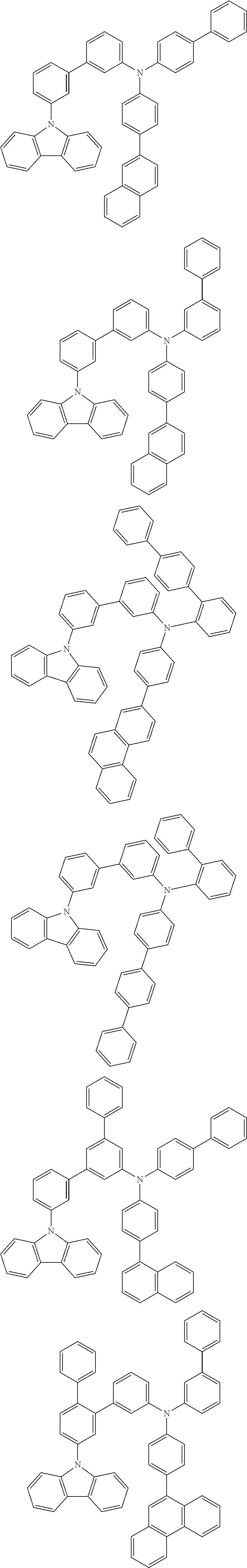

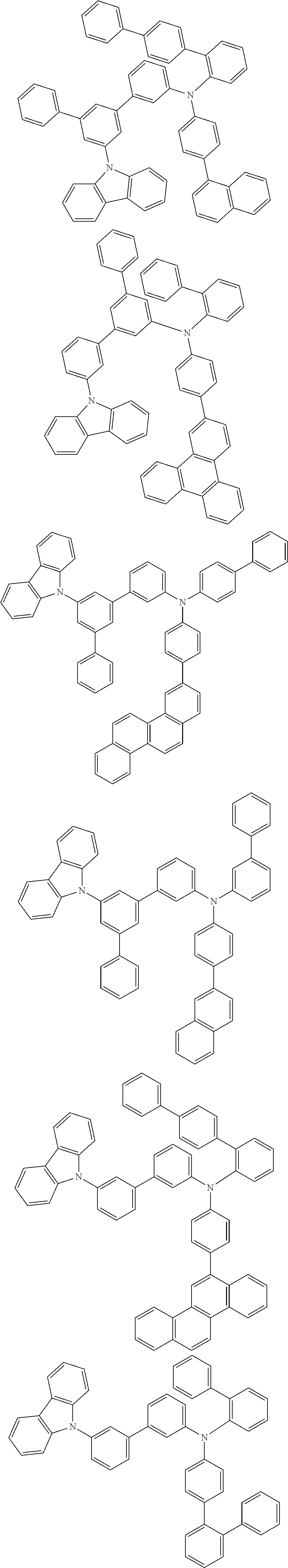

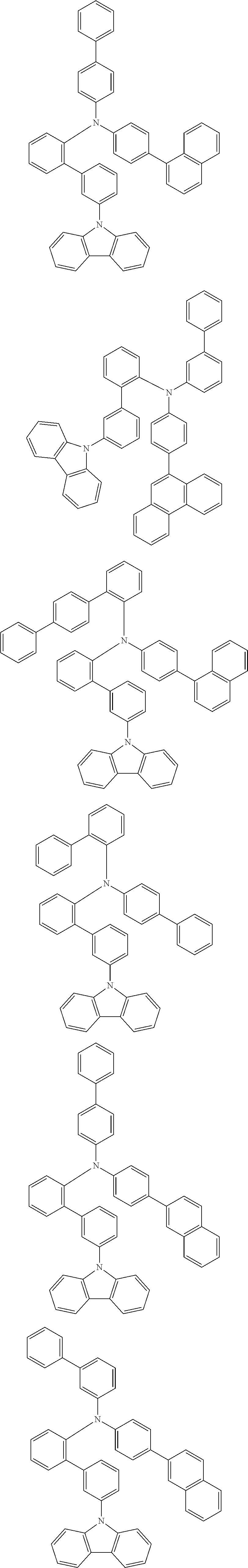

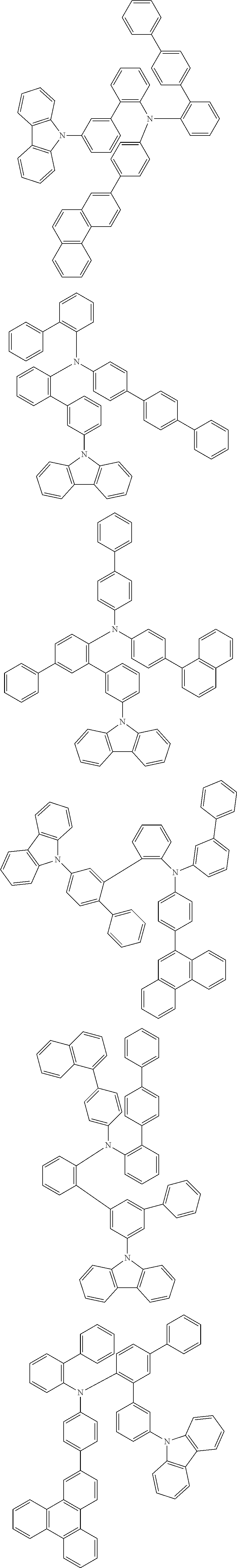

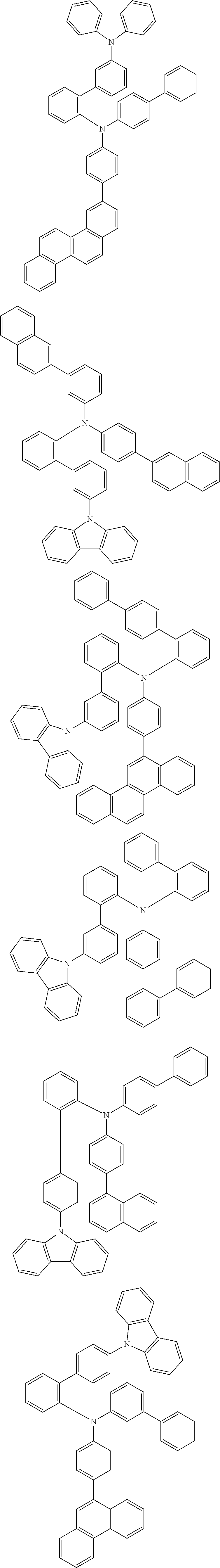

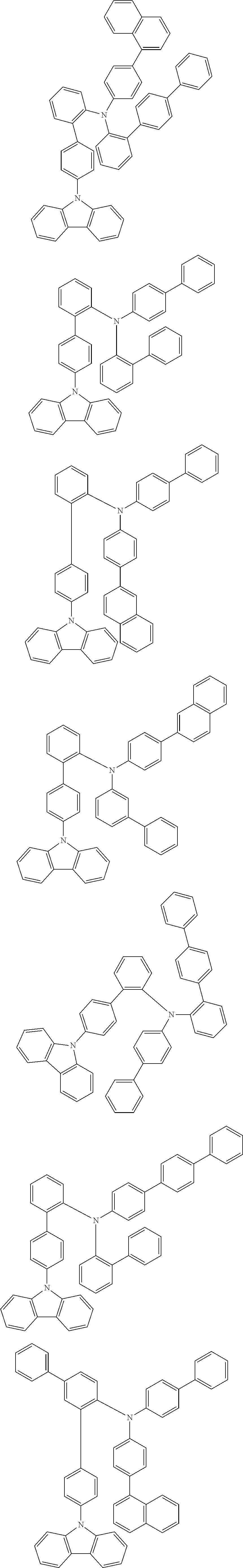

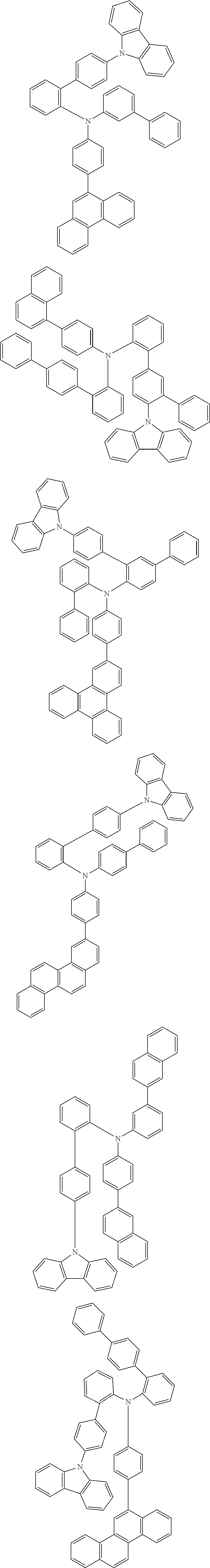

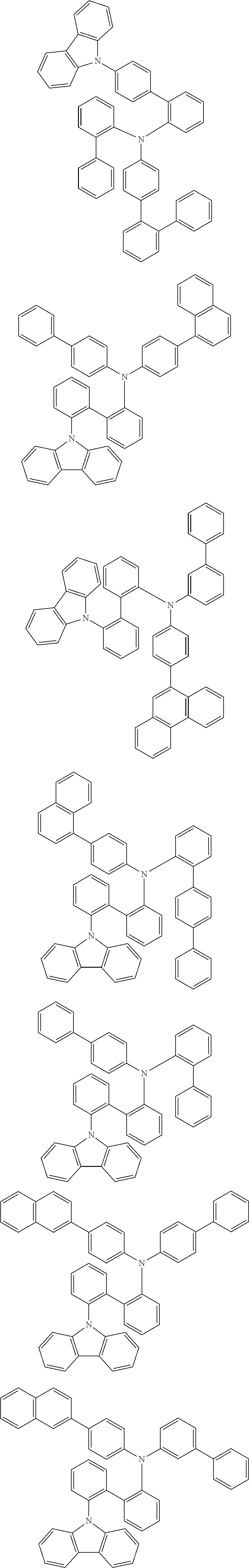

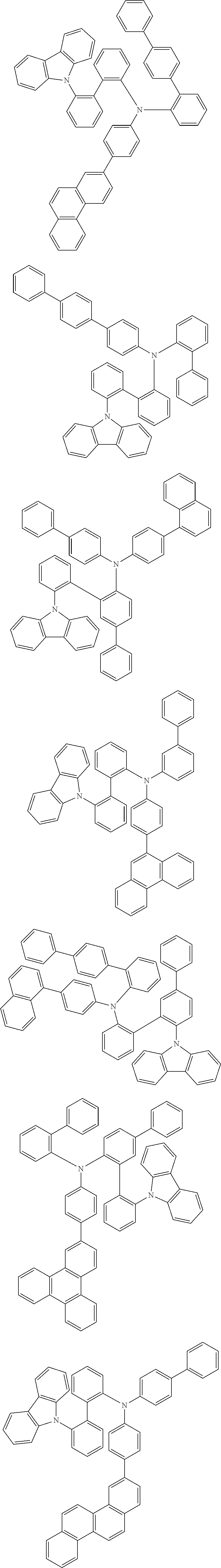

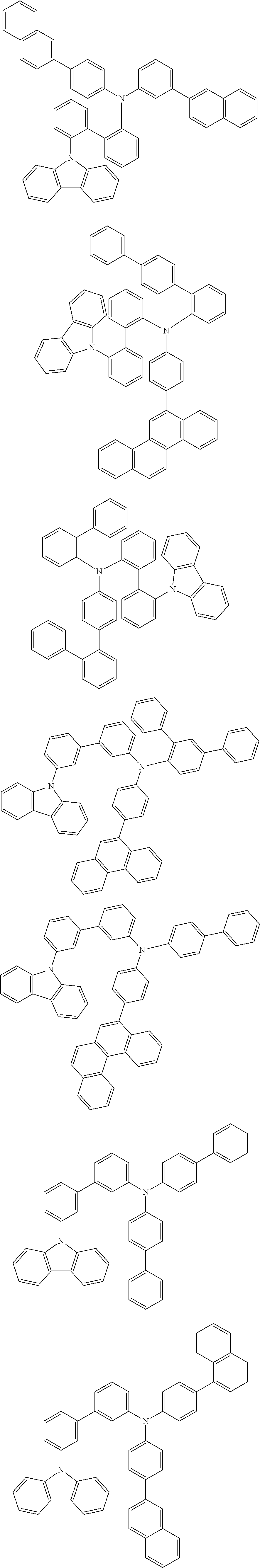

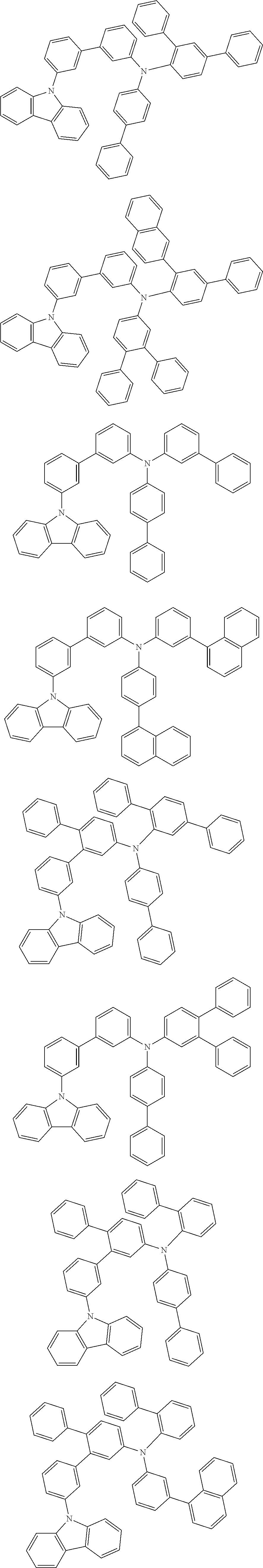

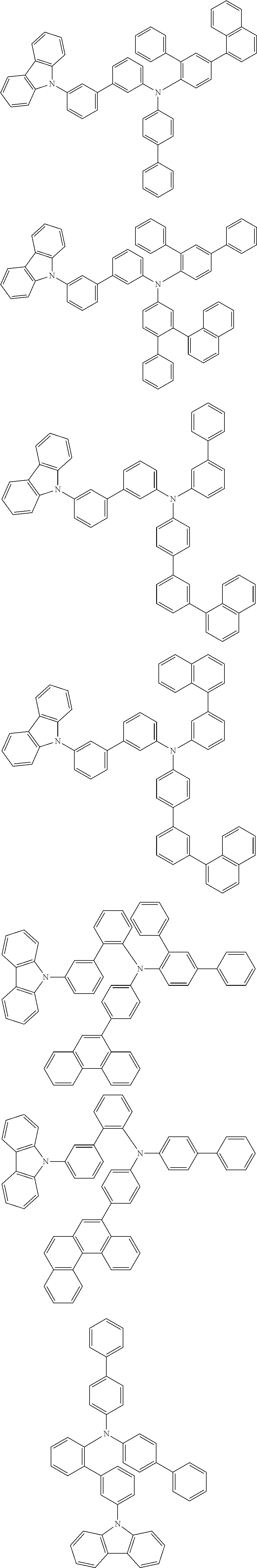

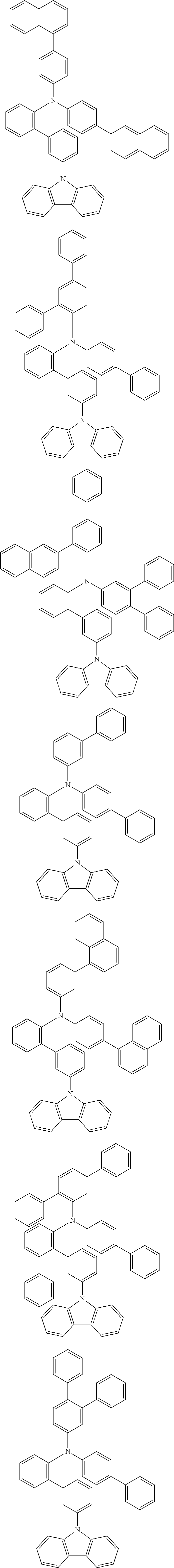

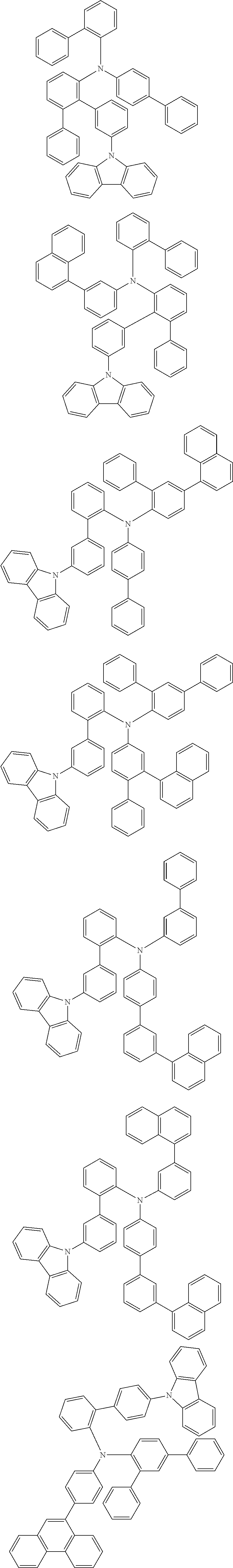

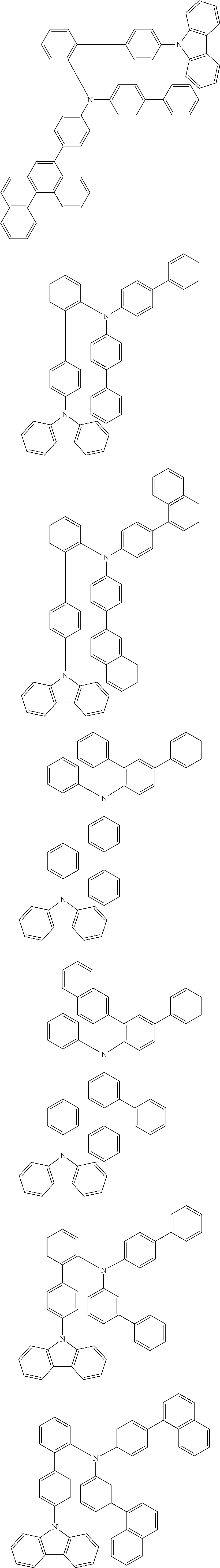

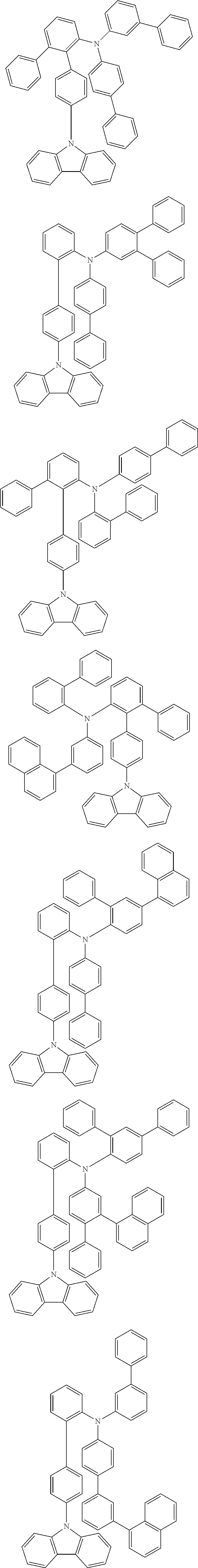

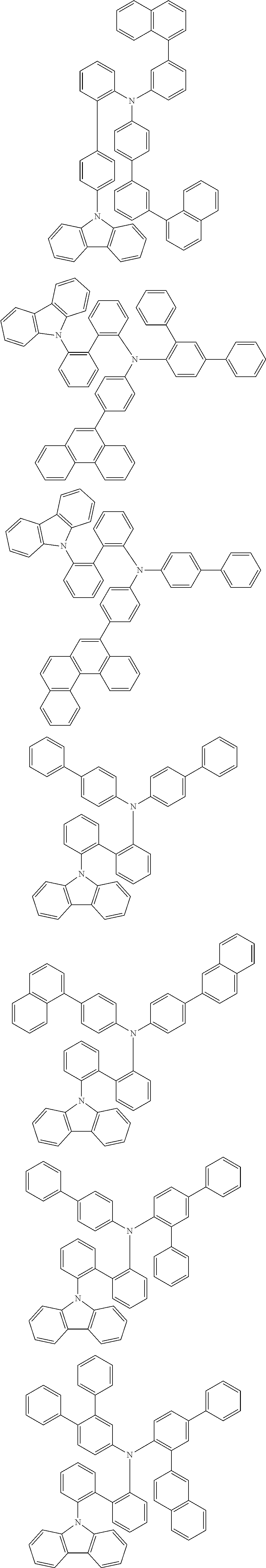

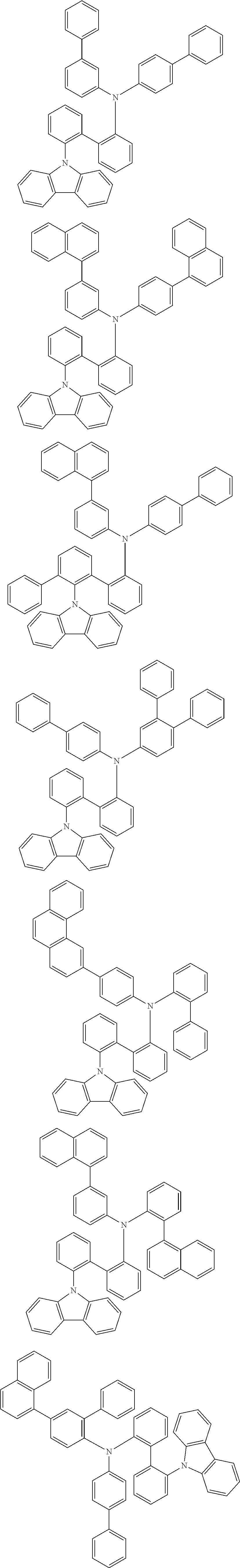

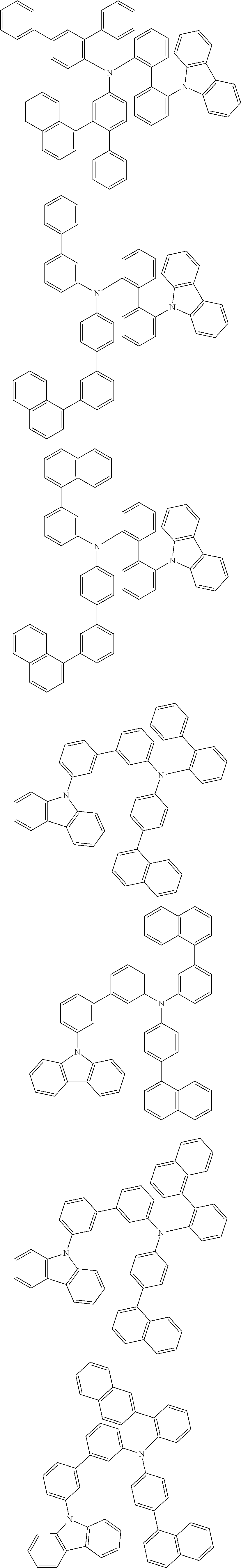

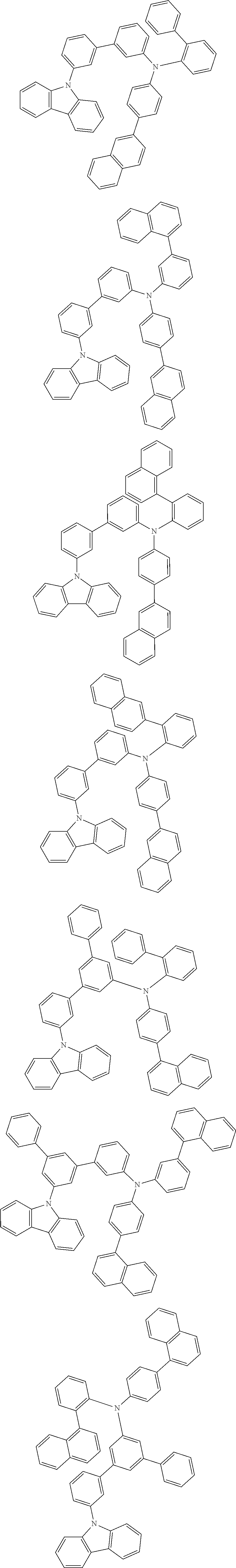

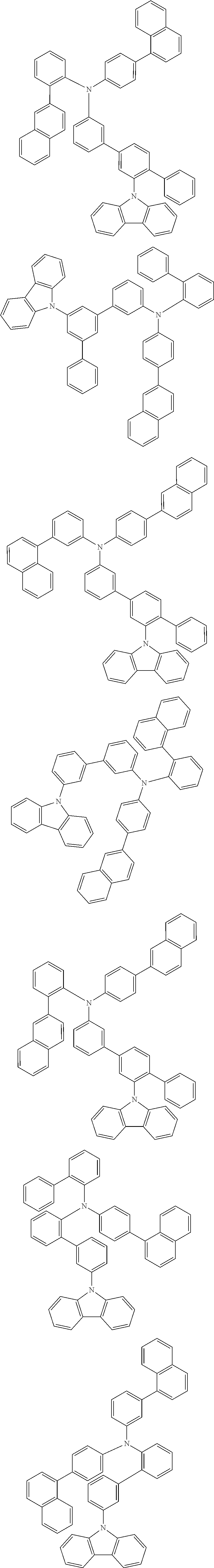

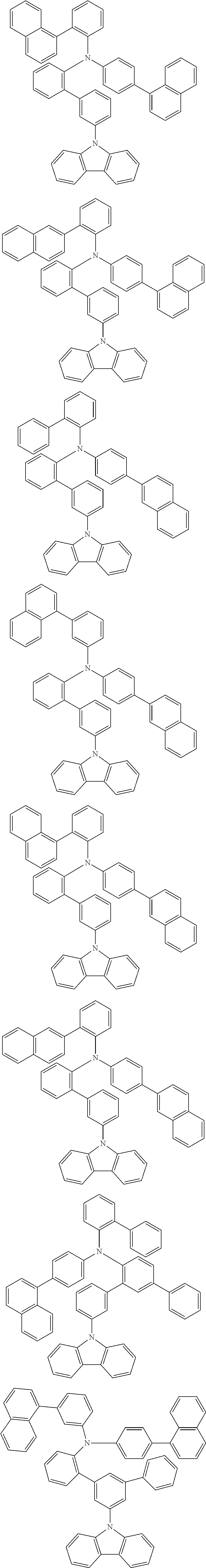

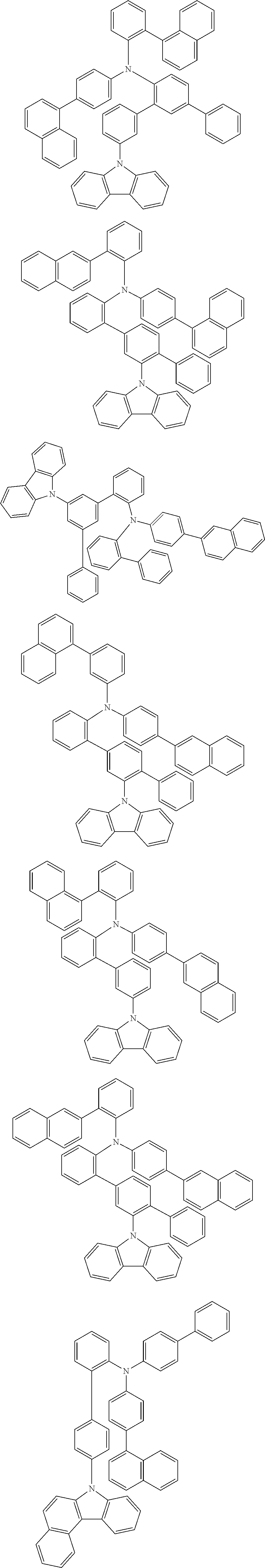

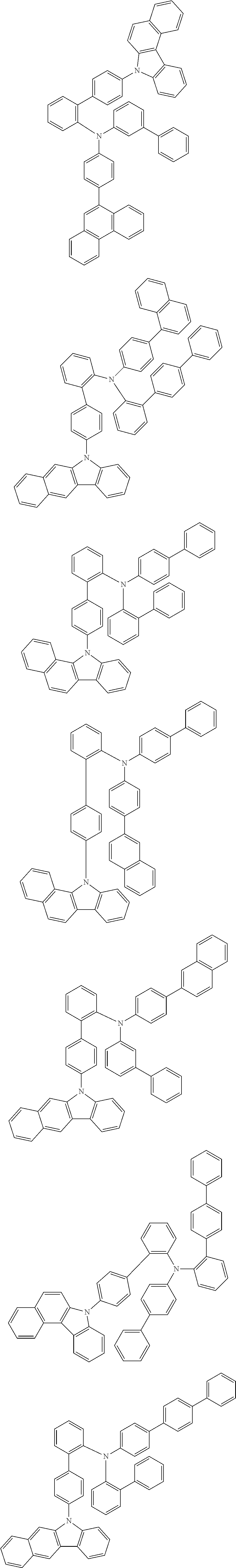

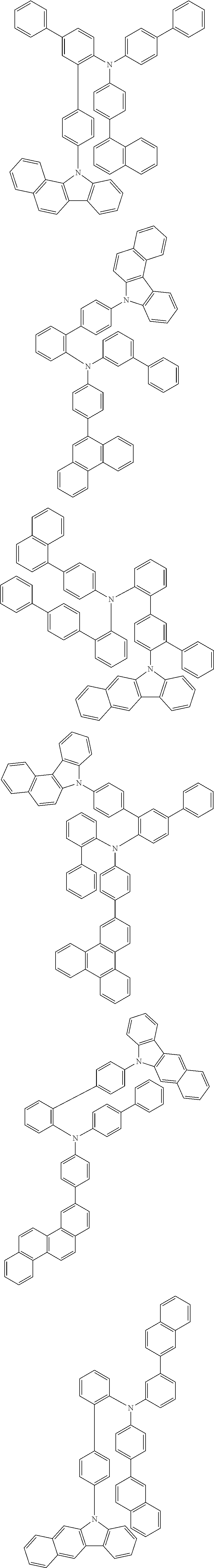

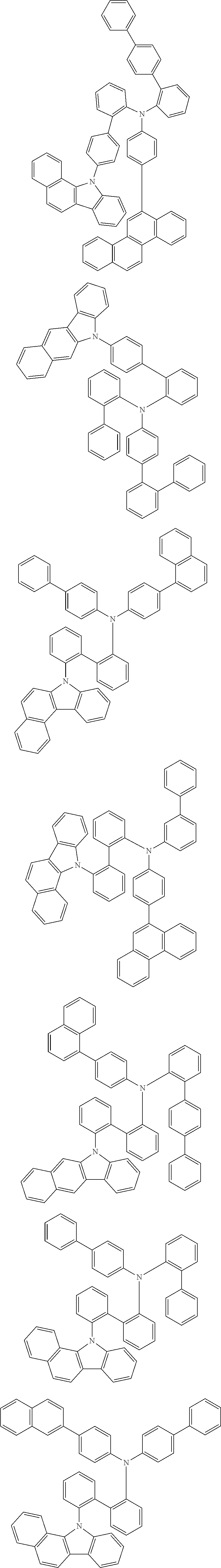

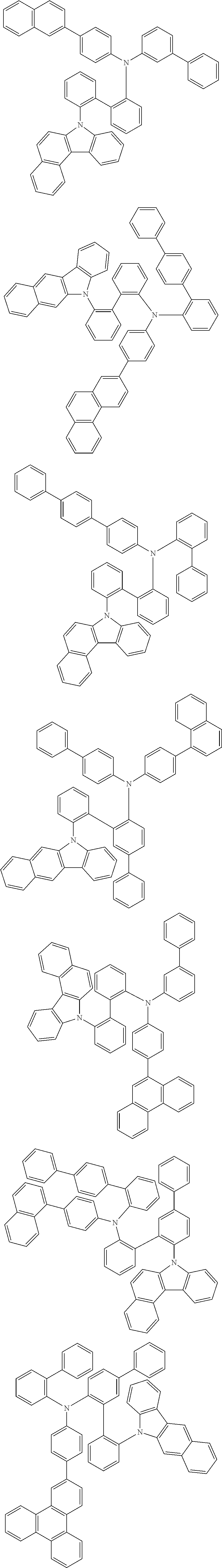

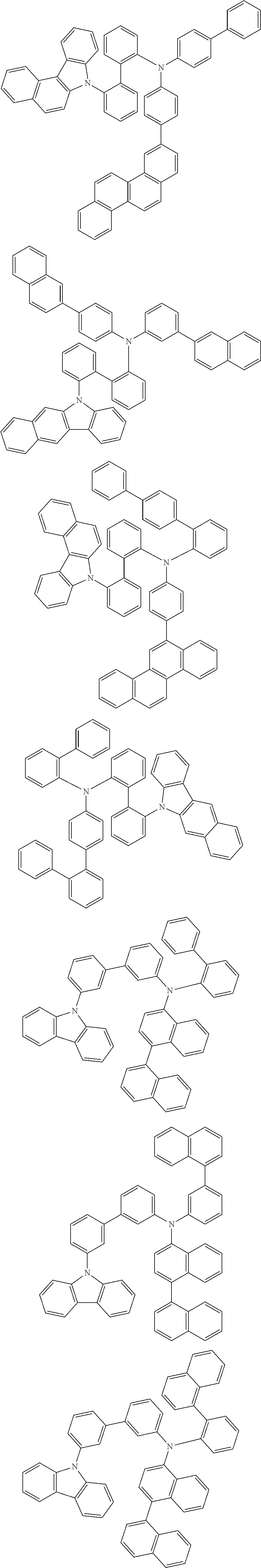

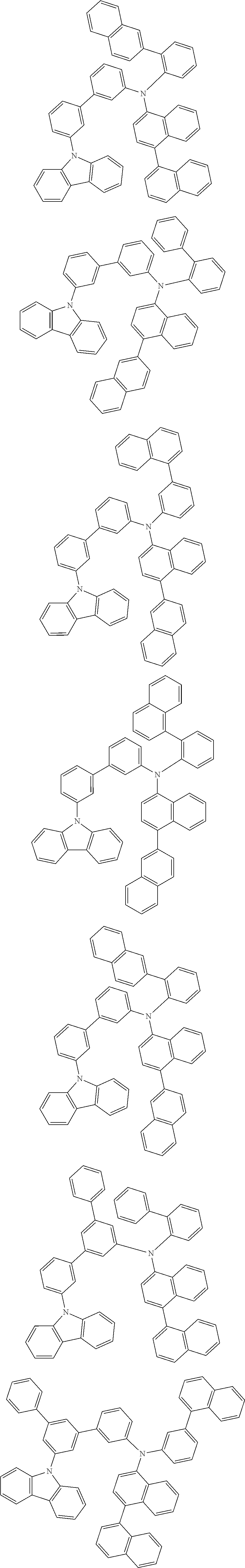

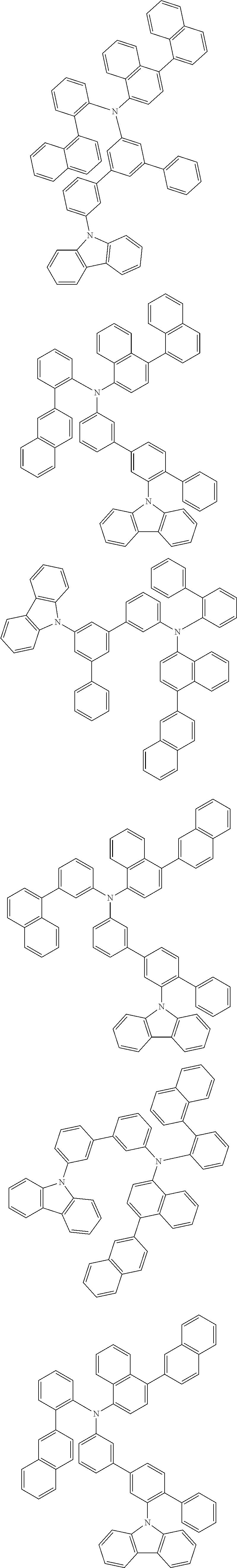

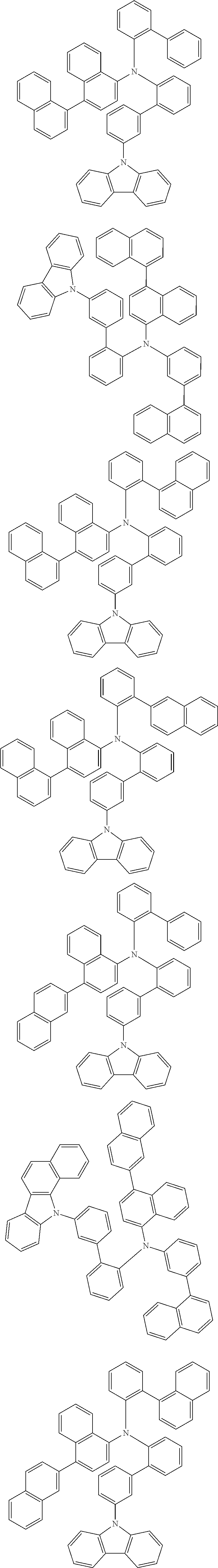

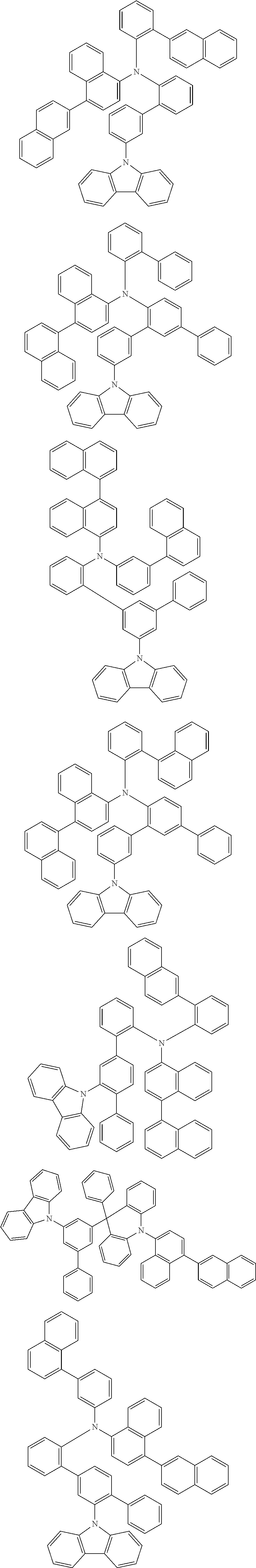

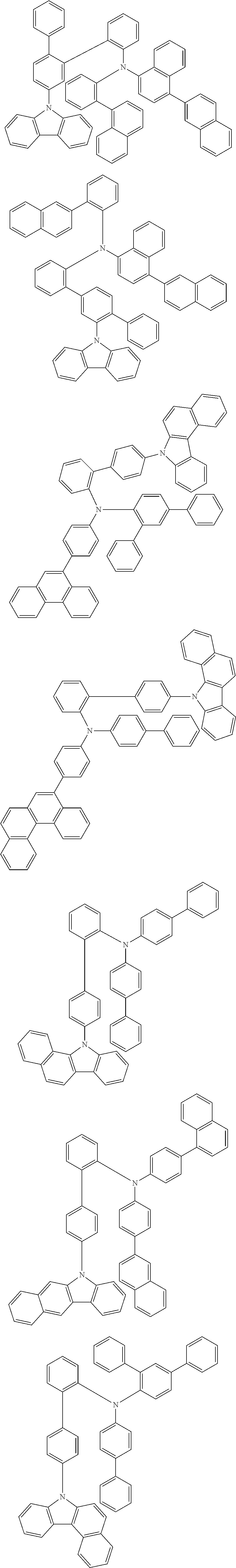

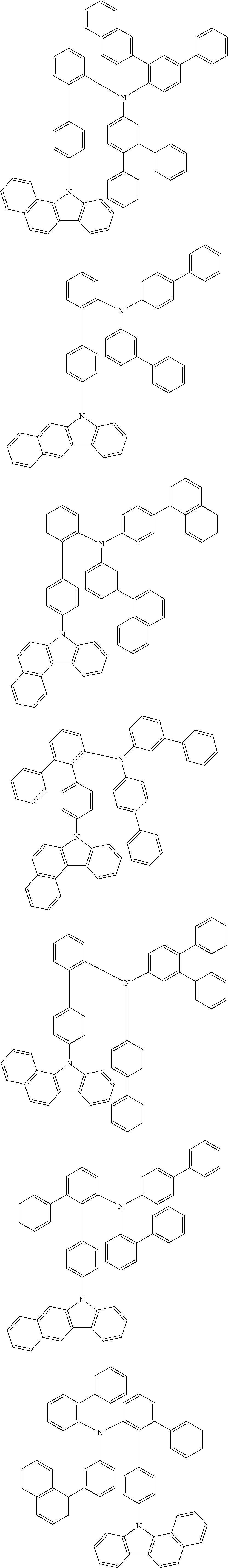

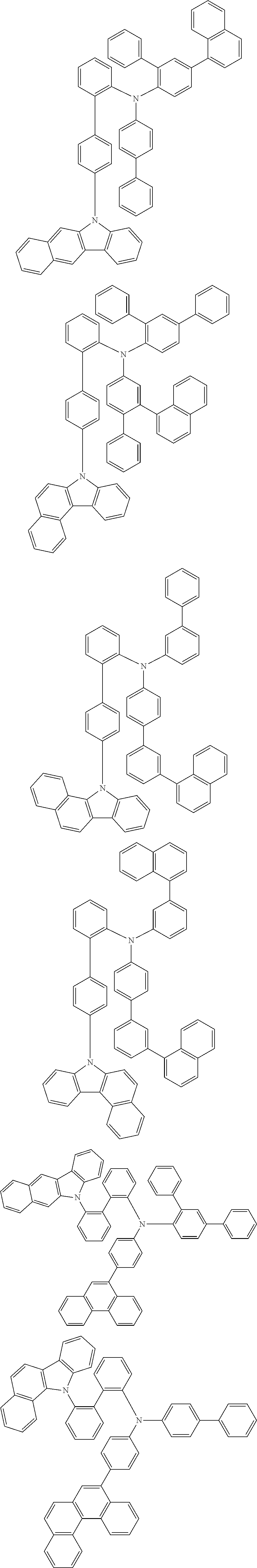

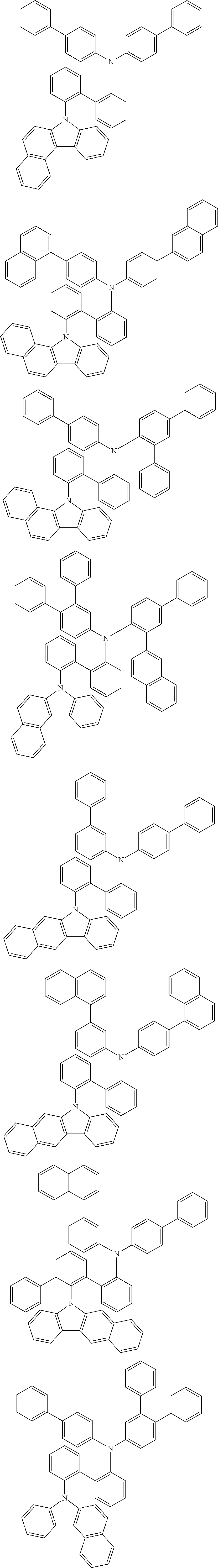

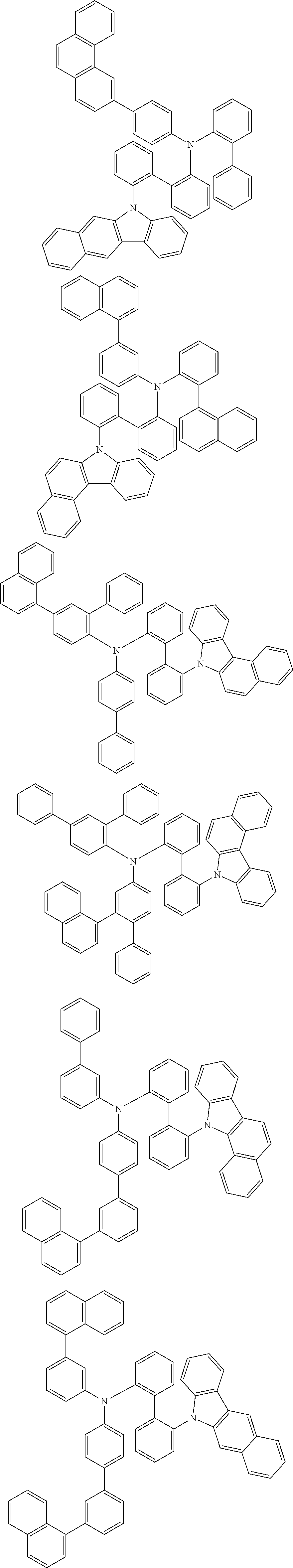

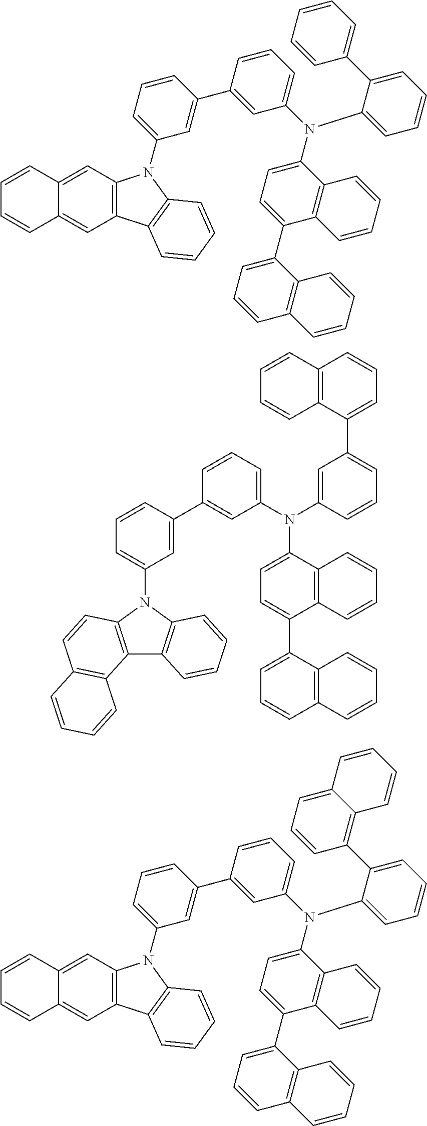

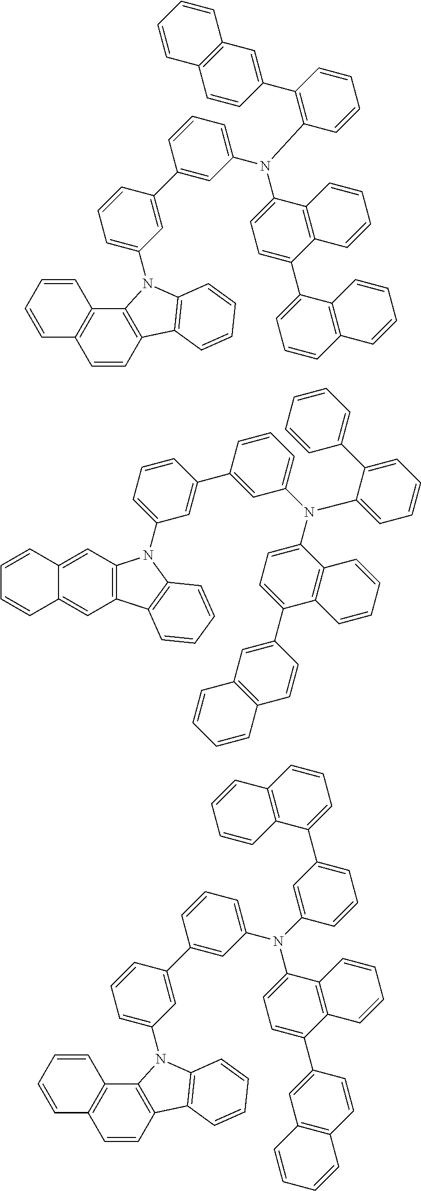

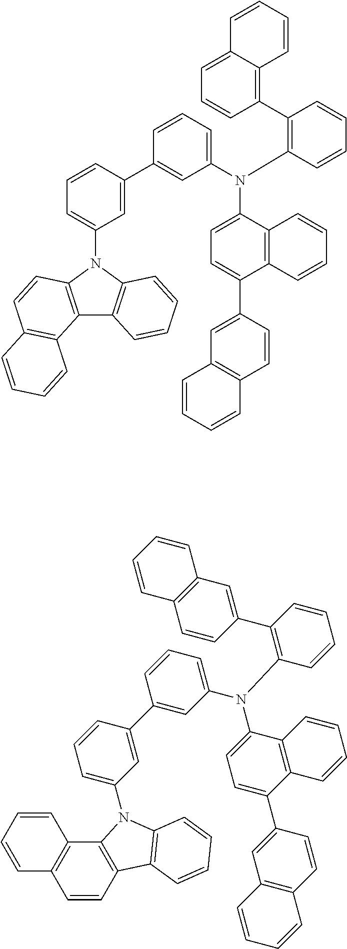 [SMILES: c1cc(-c2cccc(-n3c4ccccc4c4c5ccccc5ccc43)c2)cc(N(c2ccccc2-c2cccc3ccccc23)c2ccc(-c3ccc4ccccc4c3)c3ccccc23)c1 ; c1cc(-c2cccc(-n3c4ccccc4c4ccc5ccccc5c43)c2)cc(N(c2ccccc2-c2ccc3ccccc3c2)c2ccc(-c3ccc4ccccc4c3)c3ccccc23)c1]

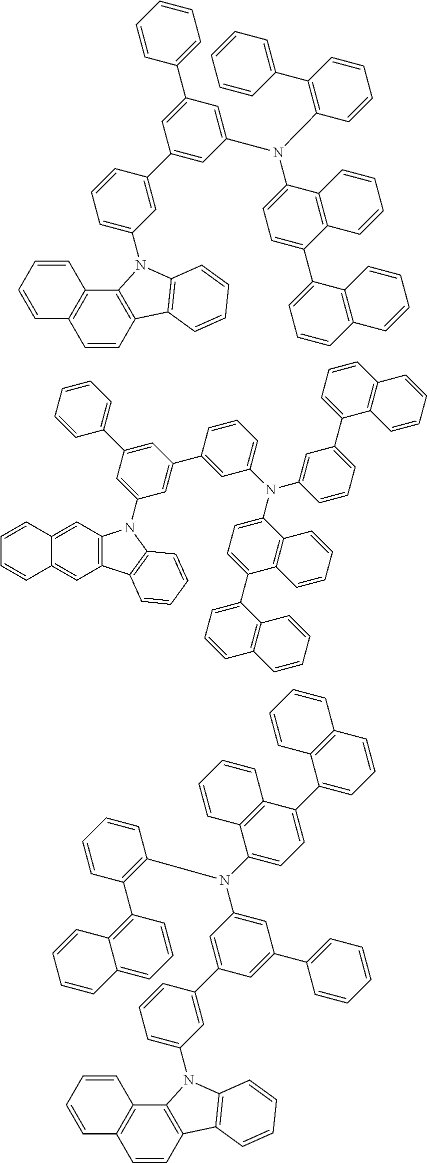

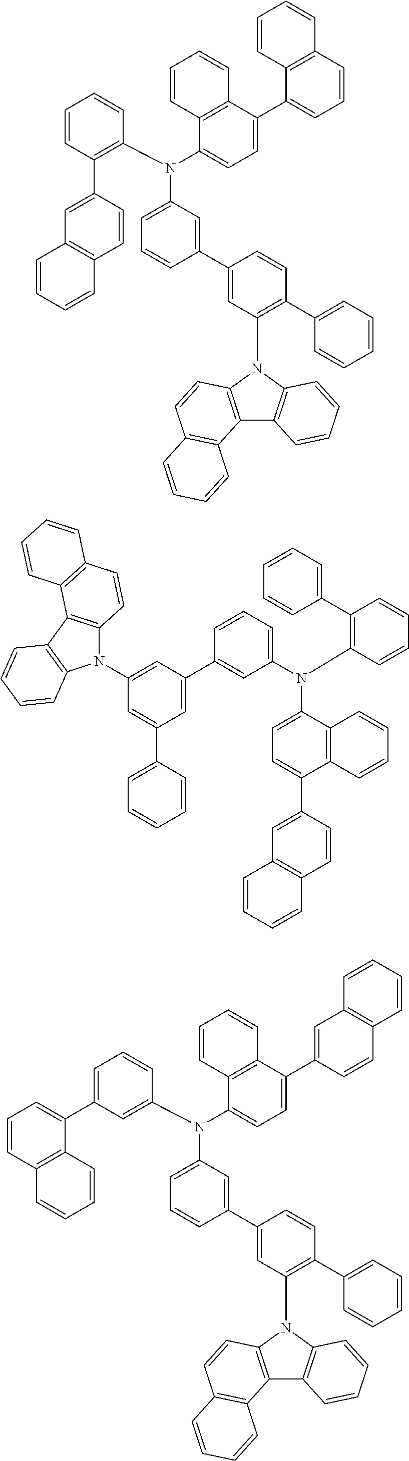 [SMILES: c1ccc(-c2ccc(-c3cccc(N(c4ccccc4-c4ccc5ccccc5c4)c4ccc(-c5cccc6ccccc56)c5ccccc45)c3)cc2-n2c3ccccc3c3c4ccccc4ccc32)cc1 ; c1ccc(-c2cc(-c3cccc(N(c4ccccc4-c4ccccc4)c4ccc(-c5ccc6ccccc6c5)c5ccccc45)c3)cc(-n3c4ccccc4c4c5ccccc5ccc43)c2)cc1 ; c1ccc(-c2ccc(-c3cccc(N(c4cccc(-c5cccc6ccccc56)c4)c4ccc(-c5ccc6ccccc6c5)c5ccccc45)c3)cc2-n2c3ccccc3c3c4ccccc4ccc32)cc1]

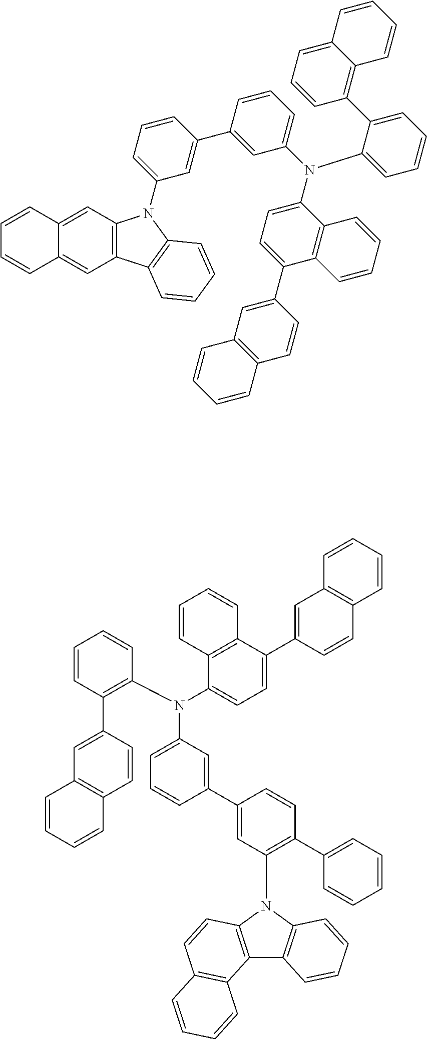 [SMILES: c1cc(-c2cccc(-n3c4ccccc4c4cc5ccccc5cc43)c2)cc(N(c2ccccc2-c2cccc3ccccc23)c2ccc(-c3ccc4ccccc4c3)c3ccccc23)c1 ; c1ccc(-c2ccc(-c3cccc(N(c4ccccc4-c4ccc5ccccc5c4)c4ccc(-c5ccc6ccccc6c5)c5ccccc45)c3)cc2-n2c3ccccc3c3c4ccccc4ccc32)cc1]

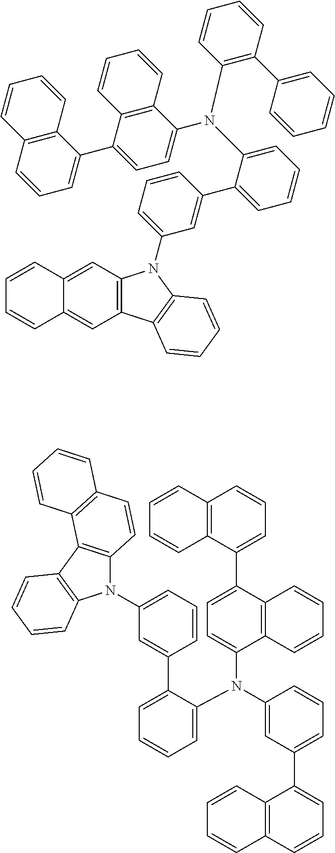 [SMILES: c1ccc(-c2ccccc2N(c2ccccc2-c2cccc(-n3c4ccccc4c4cc5ccccc5cc43)c2)c2ccc(-c3cccc4ccccc34)c3ccccc23)cc1 ; c1cc(-c2cccc3ccccc23)cc(N(c2ccccc2-c2cccc(-n3c4ccccc4c4c5ccccc5ccc43)c2)c2ccc(-c3cccc4ccccc34)c3ccccc23)c1]

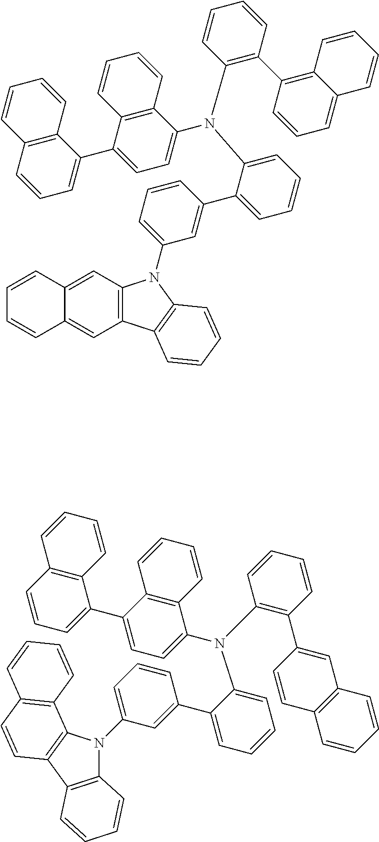 [SMILES: c1cc(-c2ccccc2N(c2ccccc2-c2cccc3ccccc23)c2ccc(-c3cccc4ccccc34)c3ccccc23)cc(-n2c3ccccc3c3cc4ccccc4cc32)c1 ; c1cc(-c2ccccc2N(c2ccccc2-c2ccc3ccccc3c2)c2ccc(-c3cccc4ccccc34)c3ccccc23)cc(-n2c3ccccc3c3ccc4ccccc4c32)c1]

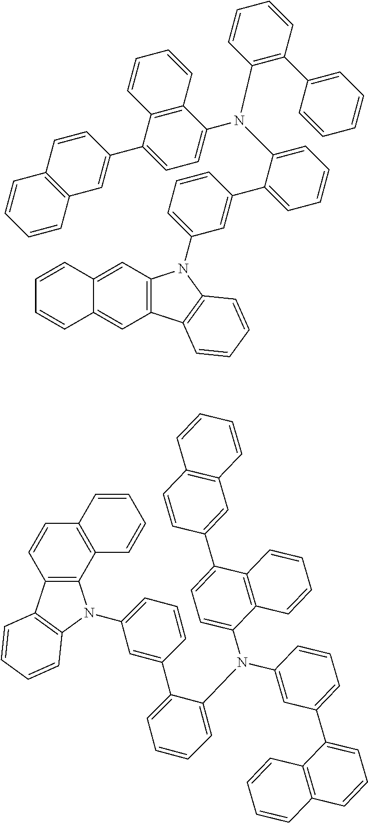 [SMILES: c1ccc(-c2ccccc2N(c2ccccc2-c2cccc(-n3c4ccccc4c4cc5ccccc5cc43)c2)c2ccc(-c3ccc4ccccc4c3)c3ccccc23)cc1 ; c1cc(-c2cccc3ccccc23)cc(N(c2ccccc2-c2cccc(-n3c4ccccc4c4ccc5ccccc5c43)c2)c2ccc(-c3ccc4ccccc4c3)c3ccccc23)c1]

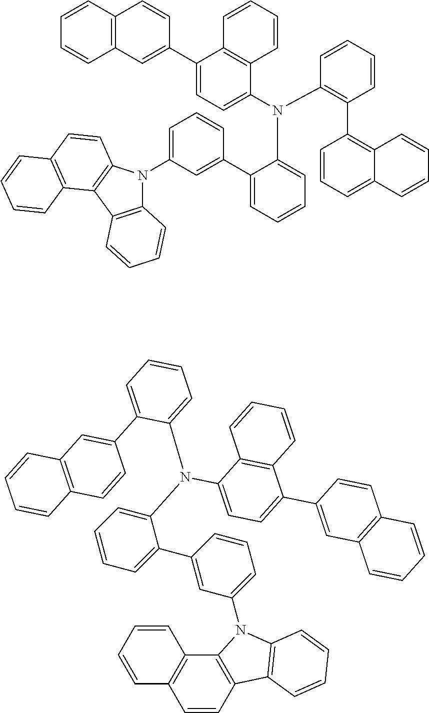 [SMILES: c1cc(-c2ccccc2N(c2ccccc2-c2cccc3ccccc23)c2ccc(-c3ccc4ccccc4c3)c3ccccc23)cc(-n2c3ccccc3c3c4ccccc4ccc32)c1 ; c1cc(-c2ccccc2N(c2ccccc2-c2ccc3ccccc3c2)c2ccc(-c3ccc4ccccc4c3)c3ccccc23)cc(-n2c3ccccc3c3ccc4ccccc4c32)c1]

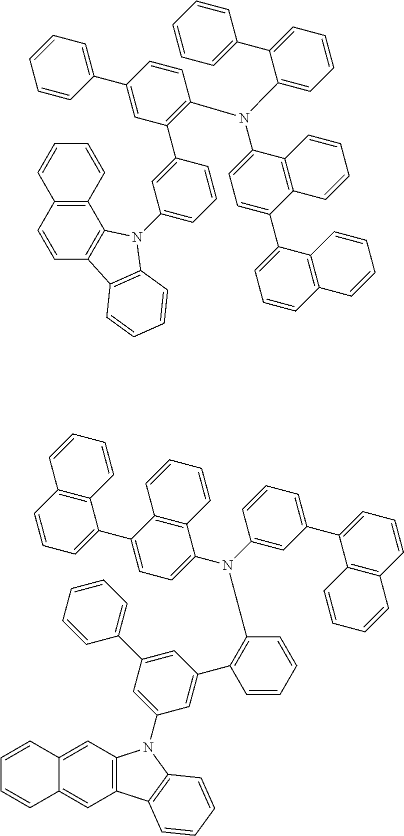 [SMILES: c1ccc(-c2ccc(N(c3ccccc3-c3ccccc3)c3ccc(-c4cccc5ccccc45)c4ccccc34)c(-c3cccc(-n4c5ccccc5c5ccc6ccccc6c54)c3)c2)cc1 ; c1ccc(-c2cc(-c3ccccc3N(c3cccc(-c4cccc5ccccc45)c3)c3ccc(-c4cccc5ccccc45)c4ccccc34)cc(-n3c4ccccc4c4cc5ccccc5cc43)c2)cc1]

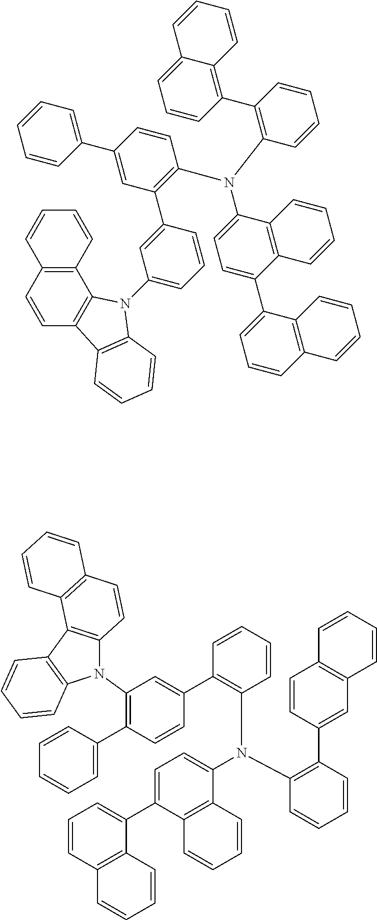 [SMILES: c1ccc(-c2ccc(N(c3ccccc3-c3cccc4ccccc34)c3ccc(-c4cccc5ccccc45)c4ccccc34)c(-c3cccc(-n4c5ccccc5c5ccc6ccccc6c54)c3)c2)cc1 ; c1ccc(-c2ccc(-c3ccccc3N(c3ccccc3-c3ccc4ccccc4c3)c3ccc(-c4cccc5ccccc45)c4ccccc34)cc2-n2c3ccccc3c3c4ccccc4ccc32)cc1]

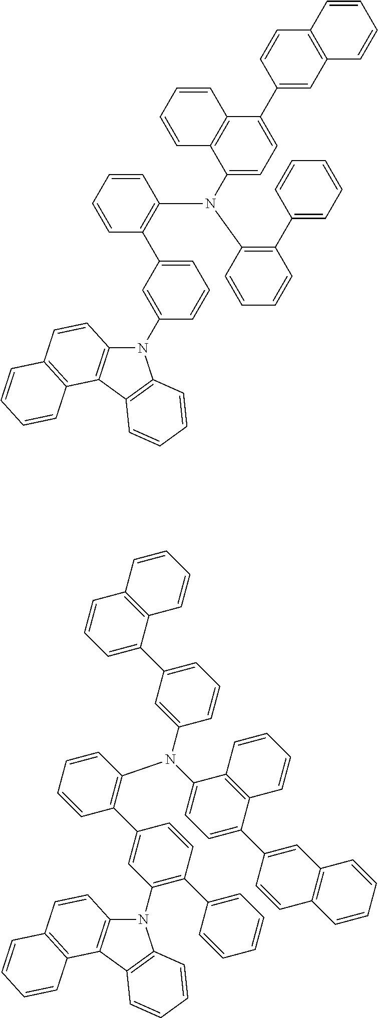 [SMILES: c1ccc(-c2ccccc2N(c2ccccc2-c2cccc(-n3c4ccccc4c4c5ccccc5ccc43)c2)c2ccc(-c3ccc4ccccc4c3)c3ccccc23)cc1 ; c1ccc(-c2ccc(-c3ccccc3N(c3cccc(-c4cccc5ccccc45)c3)c3ccc(-c4ccc5ccccc5c4)c4ccccc34)cc2-n2c3ccccc3c3c4ccccc4ccc32)cc1]

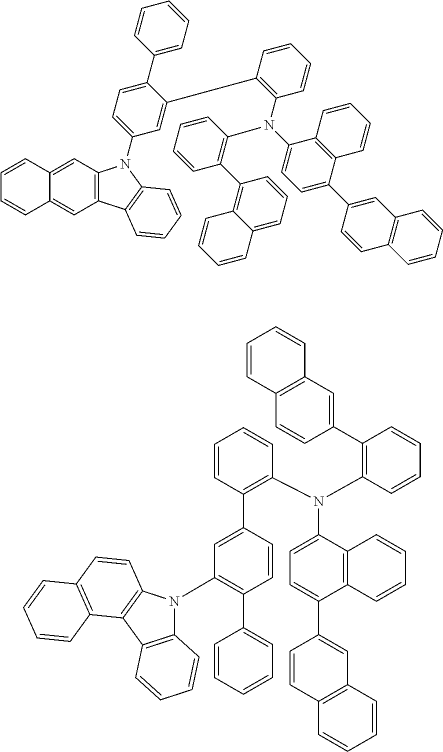 [SMILES: c1ccc(-c2ccc(-n3c4ccccc4c4cc5ccccc5cc43)cc2-c2ccccc2N(c2ccccc2-c2cccc3ccccc23)c2ccc(-c3ccc4ccccc4c3)c3ccccc23)cc1 ; c1ccc(-c2ccc(-c3ccccc3N(c3ccccc3-c3ccc4ccccc4c3)c3ccc(-c4ccc5ccccc5c4)c4ccccc34)cc2-n2c3ccccc3c3c4ccccc4ccc32)cc1]

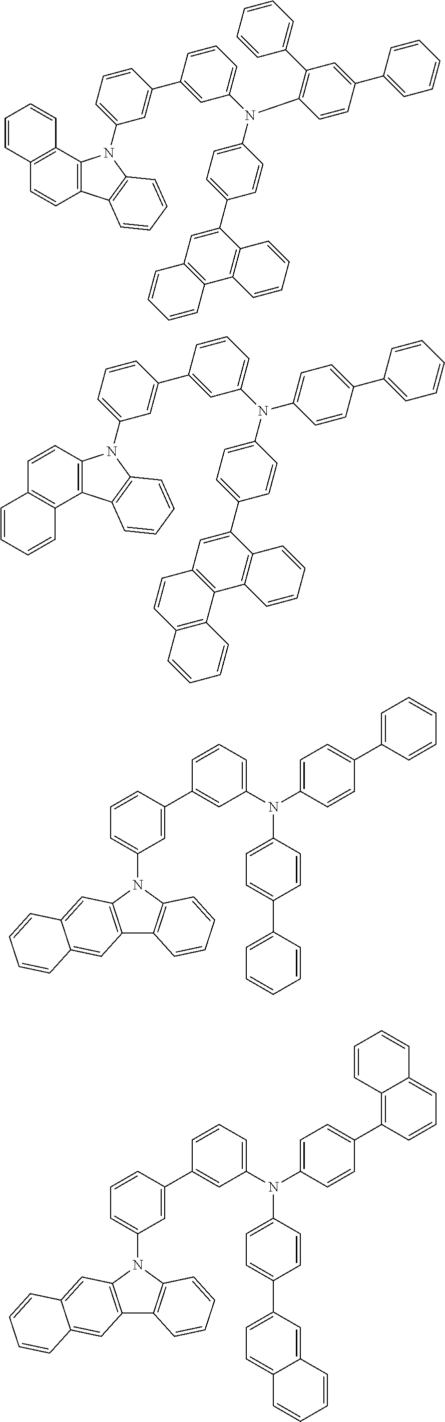

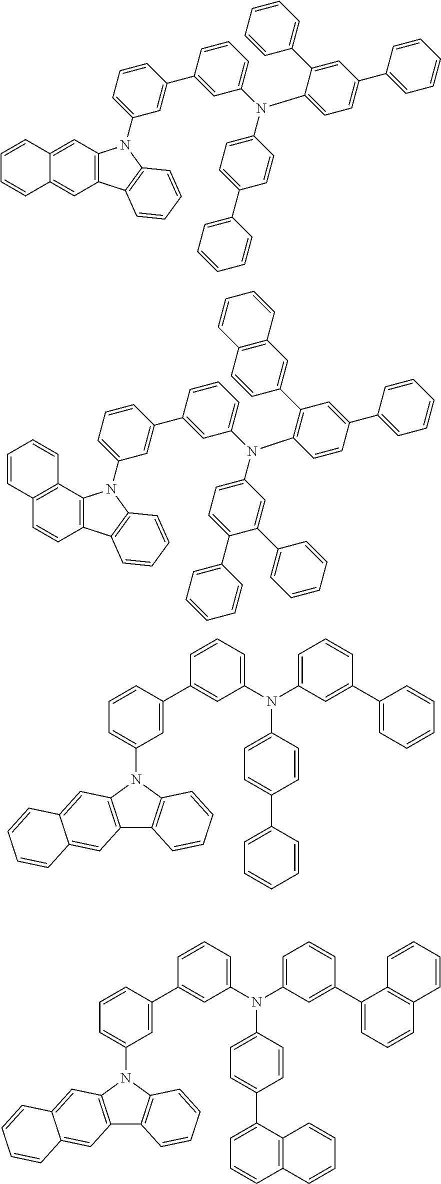

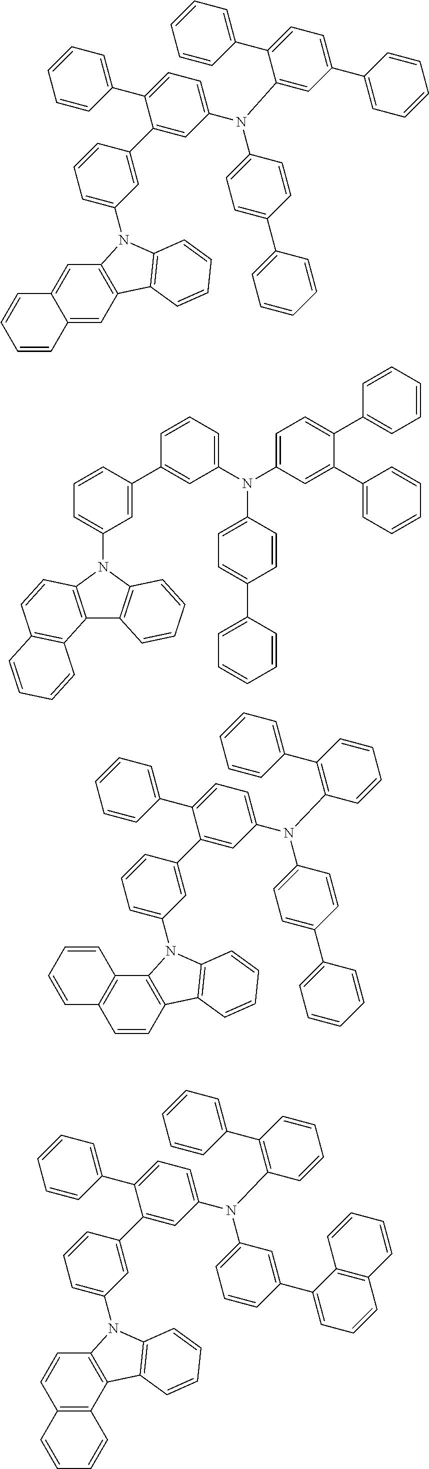

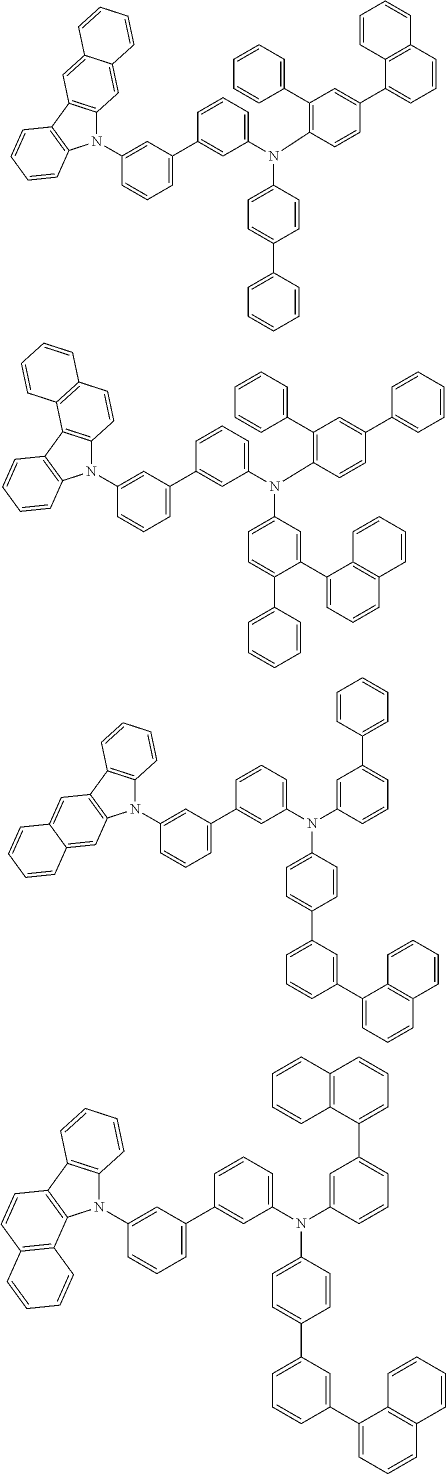

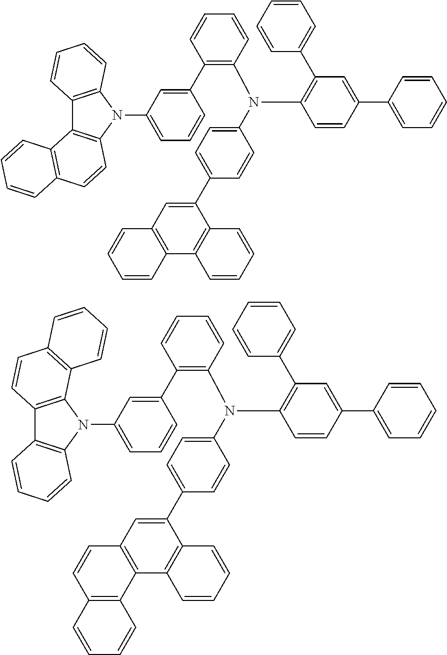

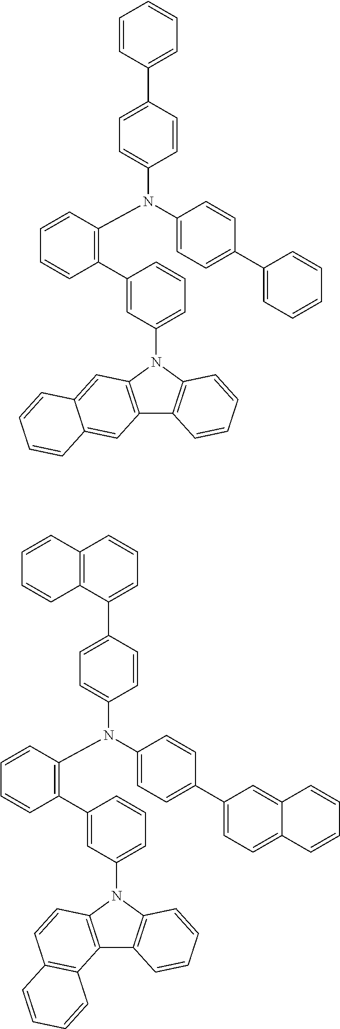 [SMILES: c1ccc(-c2ccc(N(c3ccc(-c4ccccc4)cc3)c3ccccc3-c3cccc(-n4c5ccccc5c5cc6ccccc6cc54)c3)cc2)cc1 ; c1cc(-c2ccccc2N(c2ccc(-c3ccc4ccccc4c3)cc2)c2ccc(-c3cccc4ccccc34)cc2)cc(-n2c3ccccc3c3c4ccccc4ccc32)c1]

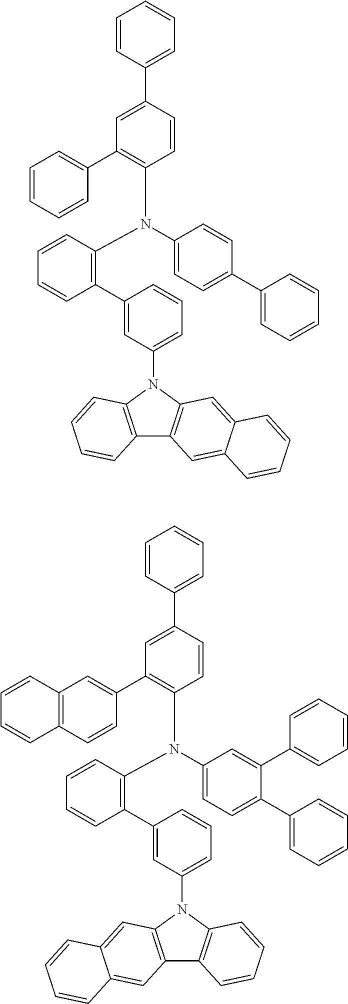 [SMILES: c1ccc(-c2ccc(N(c3ccccc3-c3cccc(-n4c5ccccc5c5cc6ccccc6cc54)c3)c3ccc(-c4ccccc4)cc3-c3ccccc3)cc2)cc1 ; c1ccc(-c2ccc(N(c3ccc(-c4ccccc4)c(-c4ccccc4)c3)c3ccccc3-c3cccc(-n4c5ccccc5c5cc6ccccc6cc54)c3)c(-c3ccc4ccccc4c3)c2)cc1]

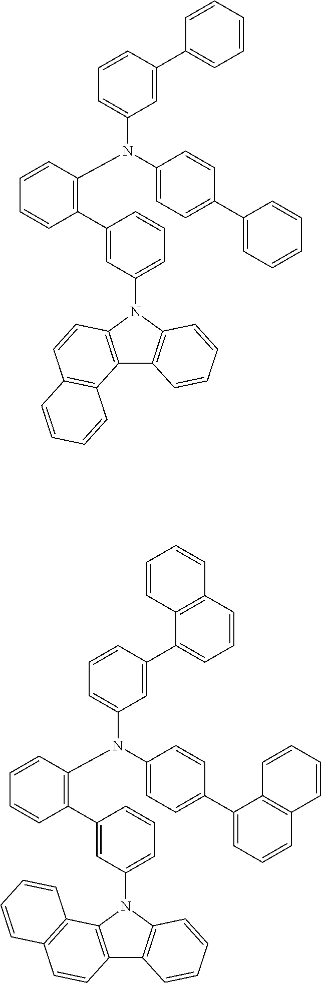 [SMILES: c1ccc(-c2ccc(N(c3cccc(-c4ccccc4)c3)c3ccccc3-c3cccc(-n4c5ccccc5c5c6ccccc6ccc54)c3)cc2)cc1 ; c1cc(-c2cccc3ccccc23)cc(N(c2ccc(-c3cccc4ccccc34)cc2)c2ccccc2-c2cccc(-n3c4ccccc4c4ccc5ccccc5c43)c2)c1]

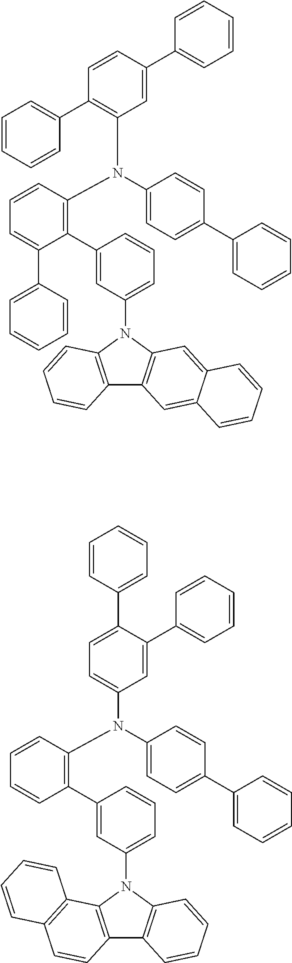 [SMILES: c1ccc(-c2ccc(N(c3cc(-c4ccccc4)ccc3-c3ccccc3)c3cccc(-c4ccccc4)c3-c3cccc(-n4c5ccccc5c5cc6ccccc6cc54)c3)cc2)cc1 ; c1ccc(-c2ccc(N(c3ccc(-c4ccccc4)c(-c4ccccc4)c3)c3ccccc3-c3cccc(-n4c5ccccc5c5ccc6ccccc6c54)c3)cc2)cc1]

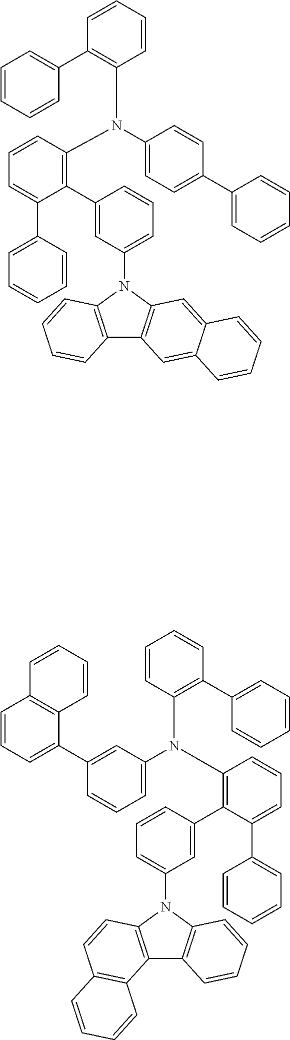 [SMILES: c1ccc(-c2ccc(N(c3ccccc3-c3ccccc3)c3cccc(-c4ccccc4)c3-c3cccc(-n4c5ccccc5c5cc6ccccc6cc54)c3)cc2)cc1 ; c1ccc(-c2ccccc2N(c2cccc(-c3cccc4ccccc34)c2)c2cccc(-c3ccccc3)c2-c2cccc(-n3c4ccccc4c4c5ccccc5ccc43)c2)cc1]

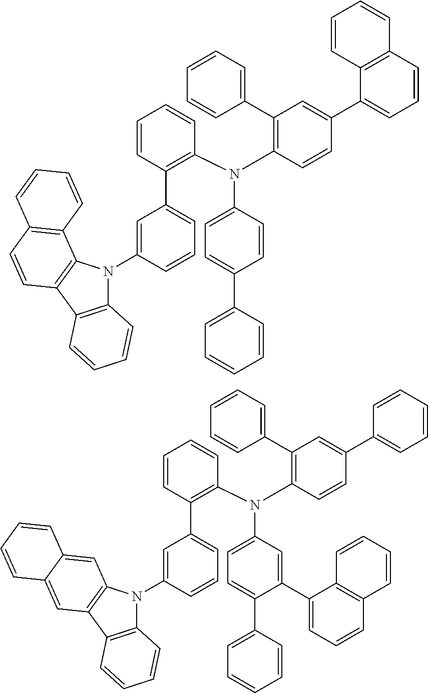

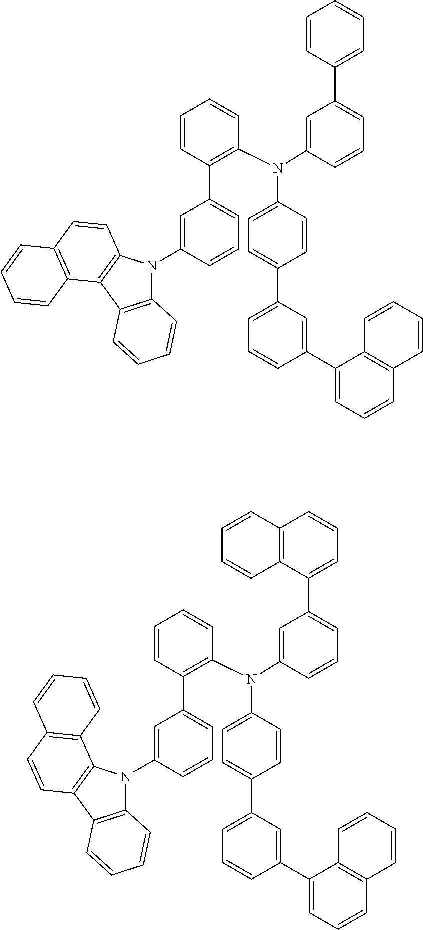 [SMILES: c1ccc(-c2cccc(N(c3ccc(-c4cccc(-c5cccc6ccccc56)c4)cc3)c3ccccc3-c3cccc(-n4c5ccccc5c5c6ccccc6ccc54)c3)c2)cc1 ; c1cc(-c2ccc(N(c3cccc(-c4cccc5ccccc45)c3)c3ccccc3-c3cccc(-n4c5ccccc5c5ccc6ccccc6c54)c3)cc2)cc(-c2cccc3ccccc23)c1]

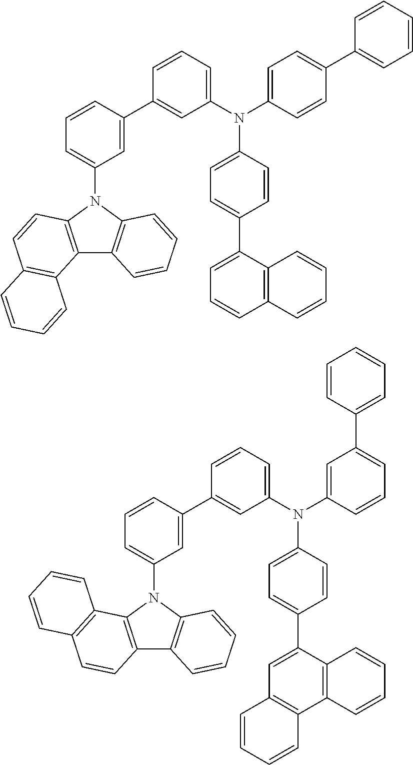 [SMILES: c1ccc(-c2ccc(N(c3ccc(-c4cccc5ccccc45)cc3)c3cccc(-c4cccc(-n5c6ccccc6c6c7ccccc7ccc65)c4)c3)cc2)cc1 ; c1ccc(-c2cccc(N(c3ccc(-c4cc5ccccc5c5ccccc45)cc3)c3cccc(-c4cccc(-n5c6ccccc6c6ccc7ccccc7c65)c4)c3)c2)cc1]

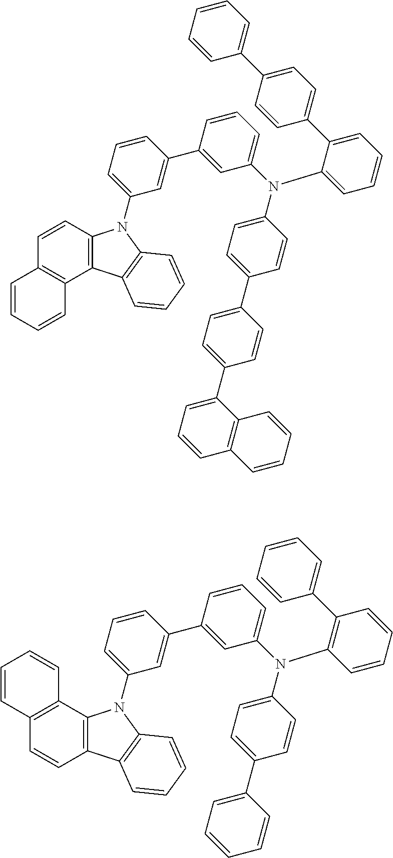 [SMILES: c1ccc(-c2ccc(-c3ccccc3N(c3ccc(-c4ccc(-c5cccc6ccccc56)cc4)cc3)c3cccc(-c4cccc(-n5c6ccccc6c6c7ccccc7ccc65)c4)c3)cc2)cc1 ; c1ccc(-c2ccc(N(c3cccc(-c4cccc(-n5c6ccccc6c6ccc7ccccc7c65)c4)c3)c3ccccc3-c3ccccc3)cc2)cc1]

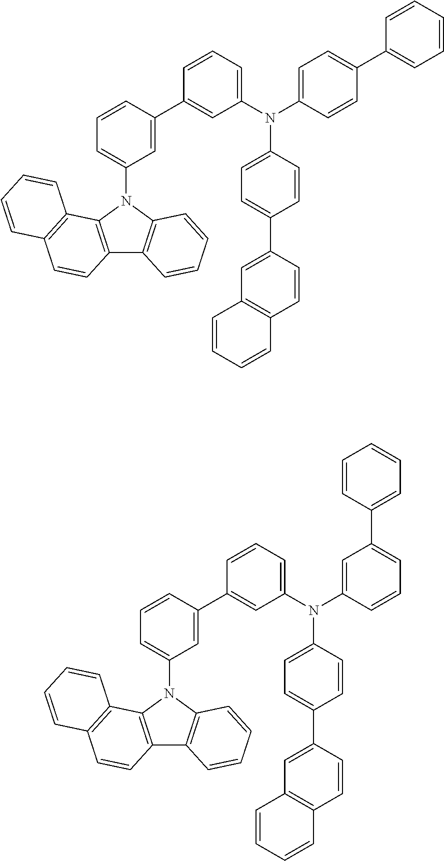 [SMILES: c1ccc(-c2ccc(N(c3ccc(-c4ccc5ccccc5c4)cc3)c3cccc(-c4cccc(-n5c6ccccc6c6ccc7ccccc7c65)c4)c3)cc2)cc1 ; c1ccc(-c2cccc(N(c3ccc(-c4ccc5ccccc5c4)cc3)c3cccc(-c4cccc(-n5c6ccccc6c6ccc7ccccc7c65)c4)c3)c2)cc1]

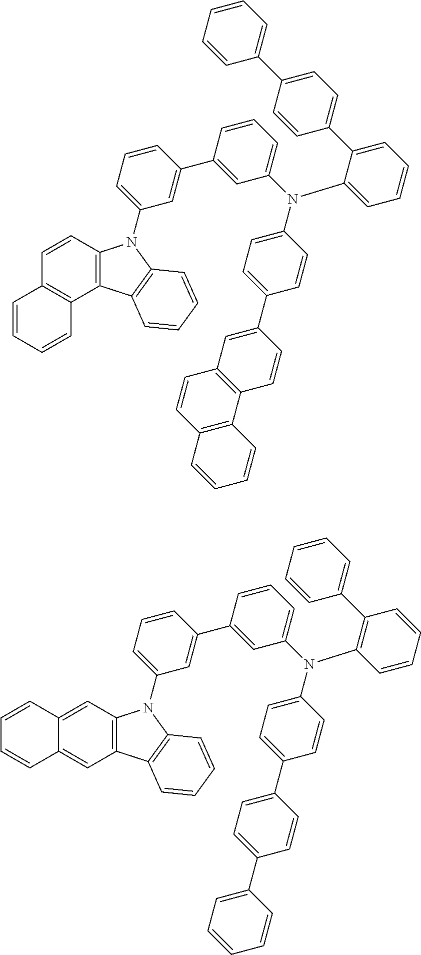 [SMILES: c1ccc(-c2ccc(-c3ccccc3N(c3ccc(-c4ccc5c(ccc6ccccc65)c4)cc3)c3cccc(-c4cccc(-n5c6ccccc6c6c7ccccc7ccc65)c4)c3)cc2)cc1 ; c1ccc(-c2ccc(-c3ccc(N(c4cccc(-c5cccc(-n6c7ccccc7c7cc8ccccc8cc76)c5)c4)c4ccccc4-c4ccccc4)cc3)cc2)cc1]

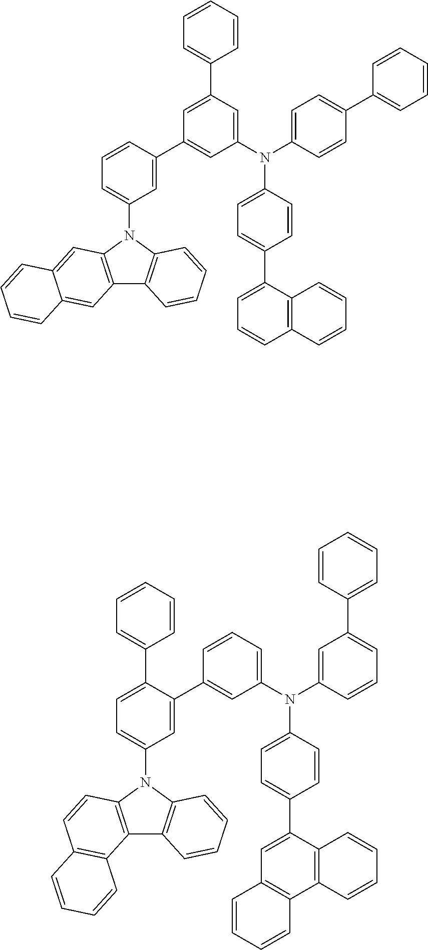 [SMILES: c1ccc(-c2ccc(N(c3ccc(-c4cccc5ccccc45)cc3)c3cc(-c4ccccc4)cc(-c4cccc(-n5c6ccccc6c6cc7ccccc7cc65)c4)c3)cc2)cc1 ; c1ccc(-c2cccc(N(c3ccc(-c4cc5ccccc5c5ccccc45)cc3)c3cccc(-c4cc(-n5c6ccccc6c6c7ccccc7ccc65)ccc4-c4ccccc4)c3)c2)cc1]

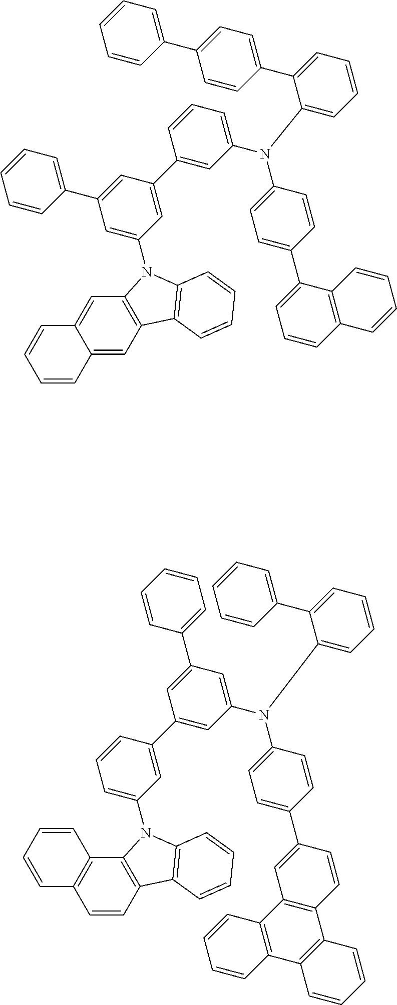 [SMILES: c1ccc(-c2ccc(-c3ccccc3N(c3ccc(-c4cccc5ccccc45)cc3)c3cccc(-c4cc(-c5ccccc5)cc(-n5c6ccccc6c6cc7ccccc7cc65)c4)c3)cc2)cc1 ; c1ccc(-c2cc(-c3cccc(-n4c5ccccc5c5ccc6ccccc6c54)c3)cc(N(c3ccc(-c4ccc5c6ccccc6c6ccccc6c5c4)cc3)c3ccccc3-c3ccccc3)c2)cc1]

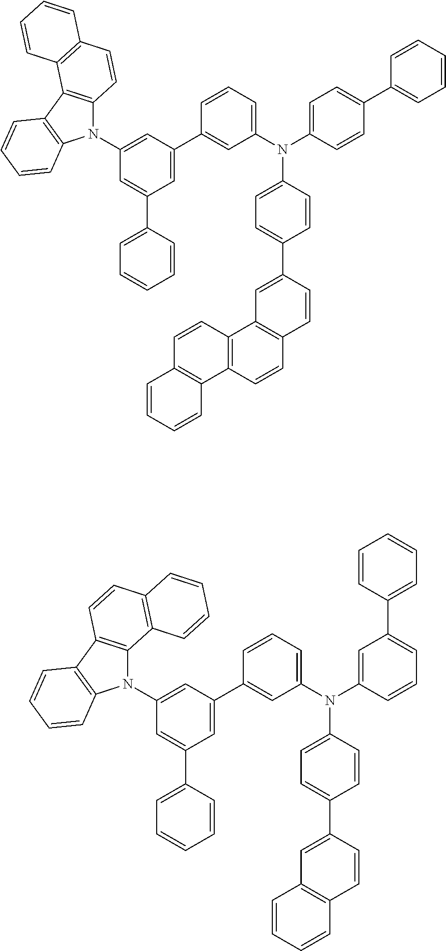 [SMILES: c1ccc(-c2ccc(N(c3ccc(-c4ccc5ccc6c7ccccc7ccc6c5c4)cc3)c3cccc(-c4cc(-c5ccccc5)cc(-n5c6ccccc6c6c7ccccc7ccc65)c4)c3)cc2)cc1 ; c1ccc(-c2cccc(N(c3ccc(-c4ccc5ccccc5c4)cc3)c3cccc(-c4cc(-c5ccccc5)cc(-n5c6ccccc6c6ccc7ccccc7c65)c4)c3)c2)cc1]

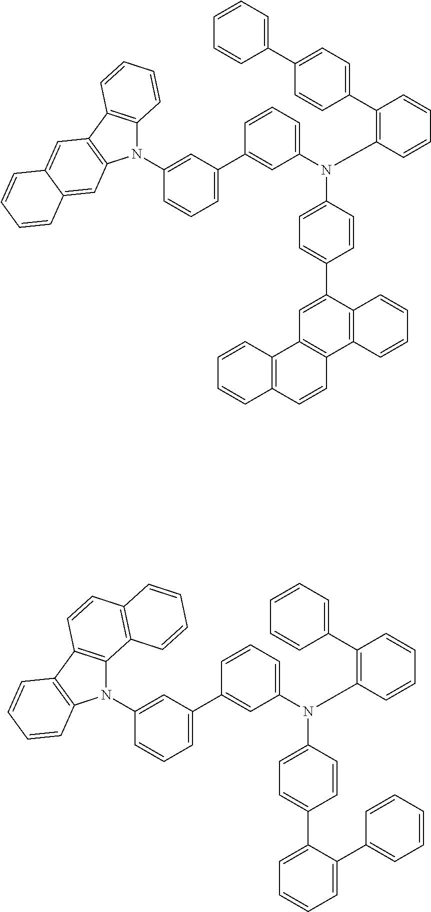 [SMILES: c1ccc(-c2ccc(-c3ccccc3N(c3ccc(-c4cc5c6ccccc6ccc5c5ccccc45)cc3)c3cccc(-c4cccc(-n5c6ccccc6c6cc7ccccc7cc65)c4)c3)cc2)cc1 ; c1ccc(-c2ccccc2-c2ccc(N(c3cccc(-c4cccc(-n5c6ccccc6c6ccc7ccccc7c65)c4)c3)c3ccccc3-c3ccccc3)cc2)cc1]

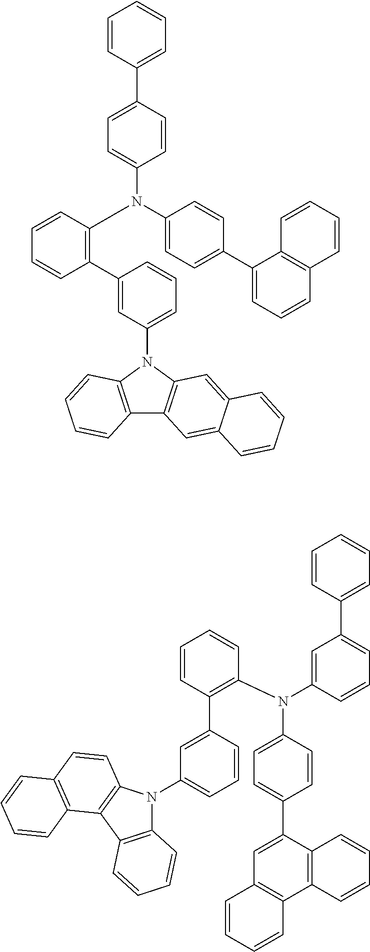 [SMILES: c1ccc(-c2ccc(N(c3ccc(-c4cccc5ccccc45)cc3)c3ccccc3-c3cccc(-n4c5ccccc5c5cc6ccccc6cc54)c3)cc2)cc1 ; c1ccc(-c2cccc(N(c3ccc(-c4cc5ccccc5c5ccccc45)cc3)c3ccccc3-c3cccc(-n4c5ccccc5c5c6ccccc6ccc54)c3)c2)cc1]

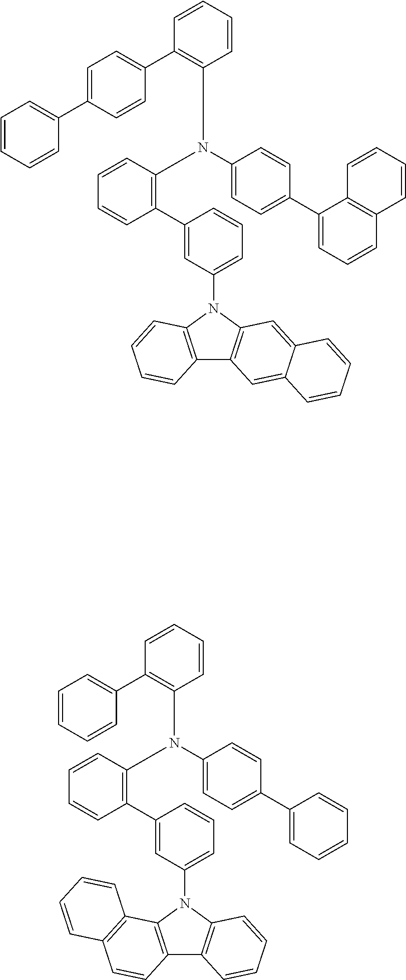 [SMILES: c1ccc(-c2ccc(-c3ccccc3N(c3ccc(-c4cccc5ccccc45)cc3)c3ccccc3-c3cccc(-n4c5ccccc5c5cc6ccccc6cc54)c3)cc2)cc1 ; c1ccc(-c2ccc(N(c3ccccc3-c3ccccc3)c3ccccc3-c3cccc(-n4c5ccccc5c5ccc6ccccc6c54)c3)cc2)cc1]

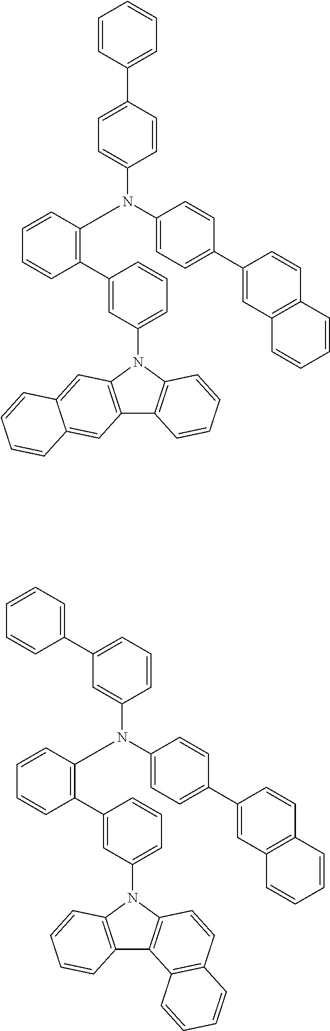 [SMILES: c1ccc(-c2ccc(N(c3ccc(-c4ccc5ccccc5c4)cc3)c3ccccc3-c3cccc(-n4c5ccccc5c5cc6ccccc6cc54)c3)cc2)cc1 ; c1ccc(-c2cccc(N(c3ccc(-c4ccc5ccccc5c4)cc3)c3ccccc3-c3cccc(-n4c5ccccc5c5c6ccccc6ccc54)c3)c2)cc1]

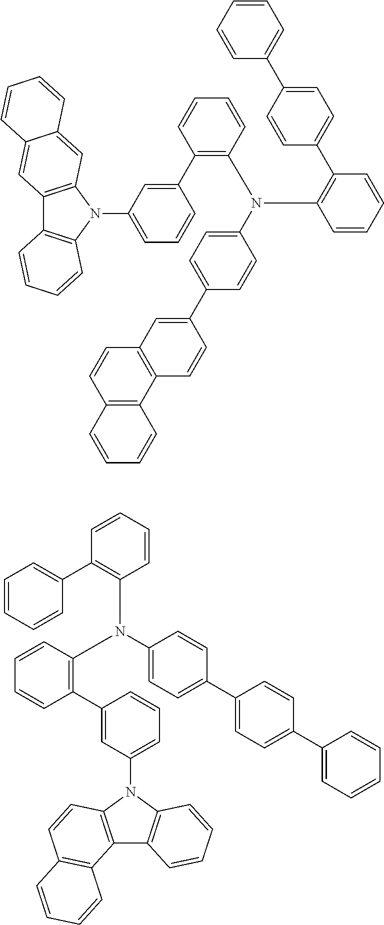 [SMILES: c1ccc(-c2ccc(-c3ccccc3N(c3ccc(-c4ccc5c(ccc6ccccc65)c4)cc3)c3ccccc3-c3cccc(-n4c5ccccc5c5cc6ccccc6cc54)c3)cc2)cc1 ; c1ccc(-c2ccc(-c3ccc(N(c4ccccc4-c4ccccc4)c4ccccc4-c4cccc(-n5c6ccccc6c6c7ccccc7ccc65)c4)cc3)cc2)cc1]

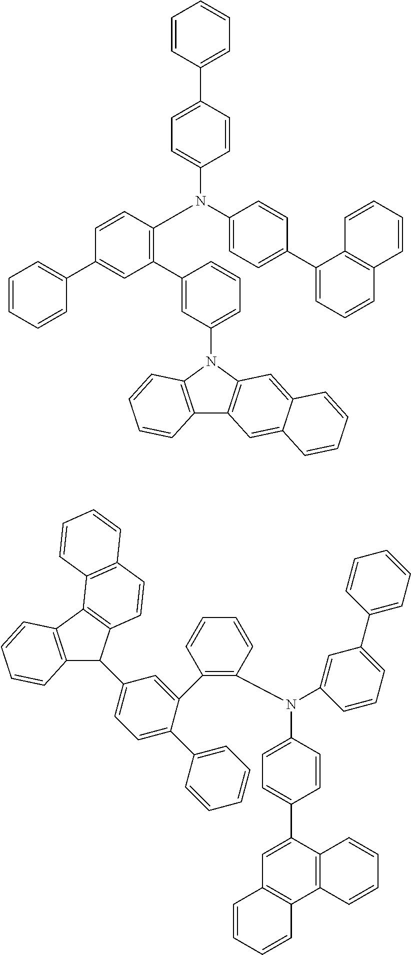 [SMILES: c1ccc(-c2ccc(N(c3ccc(-c4cccc5ccccc45)cc3)c3ccc(-c4ccccc4)cc3-c3cccc(-n4c5ccccc5c5cc6ccccc6cc54)c3)cc2)cc1 ; c1ccc(-c2cccc(N(c3ccc(-c4cc5ccccc5c5ccccc45)cc3)c3ccccc3-c3cc(C4c5ccccc5-c5c4ccc4ccccc54)ccc3-c3ccccc3)c2)cc1]

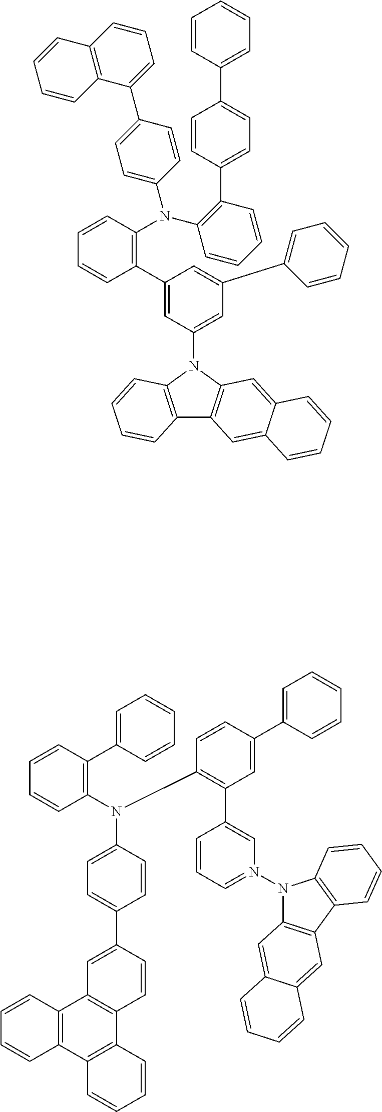

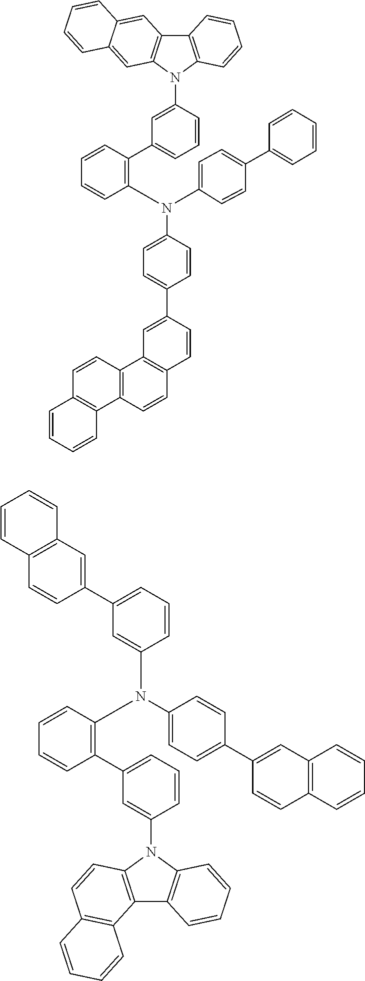 [SMILES: c1ccc(-c2ccc(N(c3ccc(-c4ccc5ccc6c7ccccc7ccc6c5c4)cc3)c3ccccc3-c3cccc(-n4c5ccccc5c5cc6ccccc6cc54)c3)cc2)cc1 ; c1cc(-c2ccc3ccccc3c2)cc(N(c2ccc(-c3ccc4ccccc4c3)cc2)c2ccccc2-c2cccc(-n3c4ccccc4c4c5ccccc5ccc43)c2)c1]

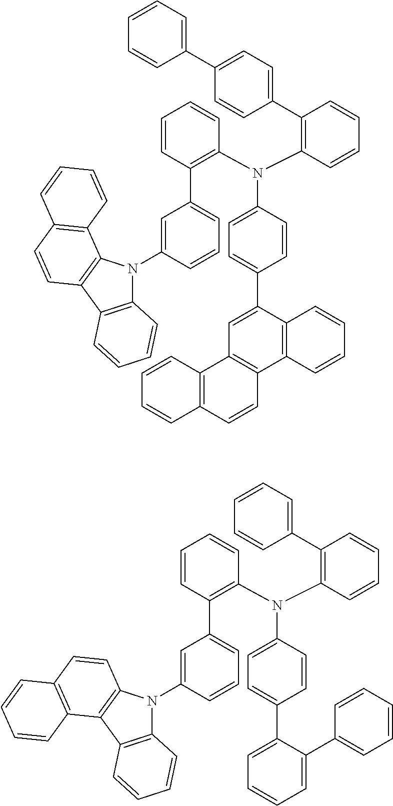 [SMILES: c1ccc(-c2ccc(-c3ccccc3N(c3ccc(-c4cc5c6ccccc6ccc5c5ccccc45)cc3)c3ccccc3-c3cccc(-n4c5ccccc5c5ccc6ccccc6c54)c3)cc2)cc1 ; c1ccc(-c2ccccc2-c2ccc(N(c3ccccc3-c3ccccc3)c3ccccc3-c3cccc(-n4c5ccccc5c5c6ccccc6ccc54)c3)cc2)cc1]

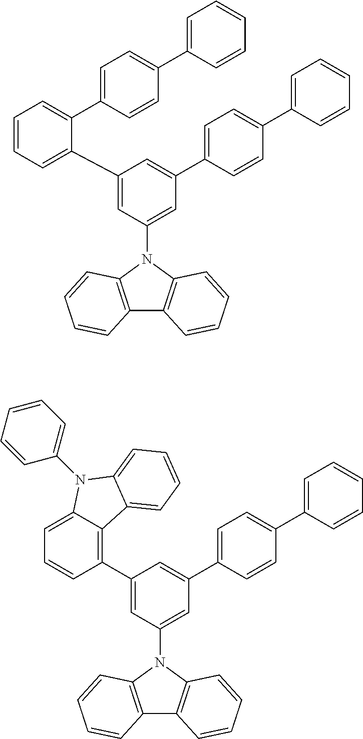 [SMILES: c1ccc(-c2ccc(-c3cc(-c4ccccc4-c4ccc(-c5ccccc5)cc4)cc(-n4c5ccccc5c5ccccc54)c3)cc2)cc1 ; c1ccc(-c2ccc(-c3cc(-c4cccc5c4c4ccccc4n5-c4ccccc4)cc(-n4c5ccccc5c5ccccc54)c3)cc2)cc1]

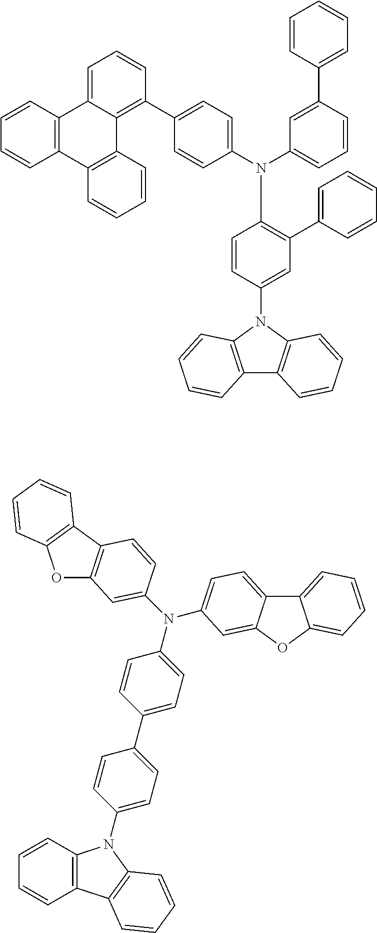 [SMILES: c1ccc(-c2cccc(N(c3ccc(-c4cccc5c6ccccc6c6ccccc6c45)cc3)c3ccc(-n4c5ccccc5c5ccccc54)cc3-c3ccccc3)c2)cc1 ; c1ccc2c(c1)oc1cc(N(c3ccc(-c4ccc(-n5c6ccccc6c6ccccc65)cc4)cc3)c3ccc4c(c3)oc3ccccc34)ccc12]

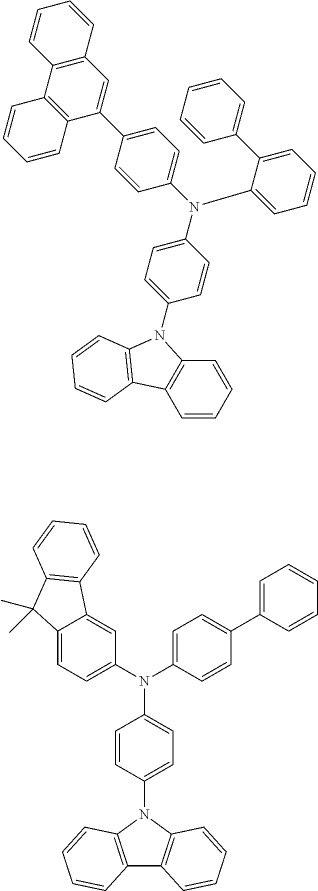 [SMILES: c1ccc(-c2ccccc2N(c2ccc(-c3cc4ccccc4c4ccccc34)cc2)c2ccc(-n3c4ccccc4c4ccccc43)cc2)cc1 ; CC1(C)c2ccccc2-c2cc(N(c3ccc(-c4ccccc4)cc3)c3ccc(-n4c5ccccc5c5ccccc54)cc3)ccc21]

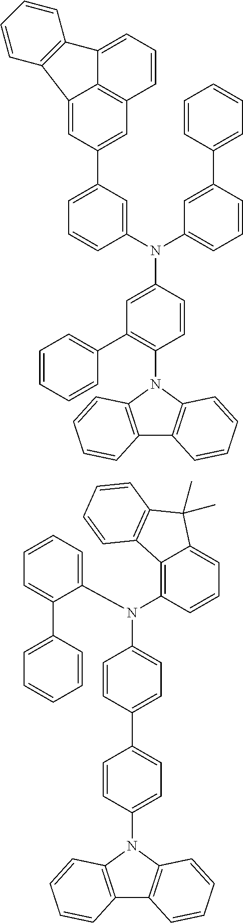

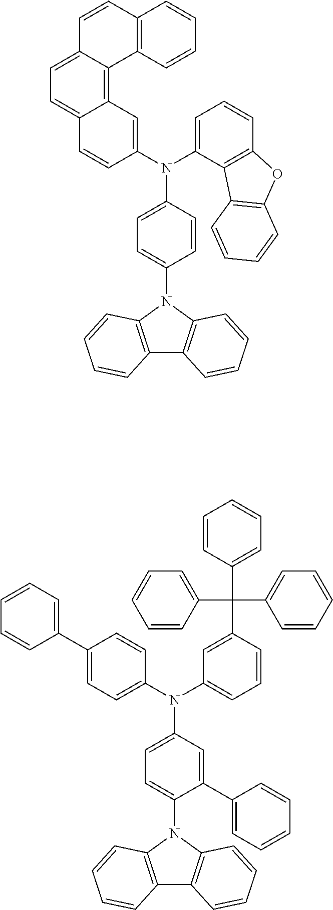 [SMILES: c1ccc2c(c1)ccc1ccc3ccc(N(c4ccc(-n5c6ccccc6c6ccccc65)cc4)c4cccc5oc6ccccc6c45)cc3c12 ; c1ccc(-c2ccc(N(c3cccc(C(c4ccccc4)(c4ccccc4)c4ccccc4)c3)c3ccc(-n4c5ccccc5c5ccccc54)c(-c4ccccc4)c3)cc2)cc1]

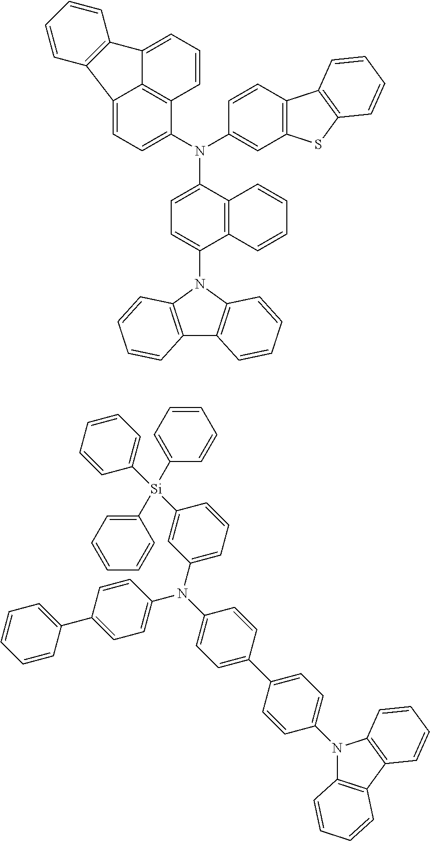 [SMILES: c1ccc2c(c1)-c1cccc3c(N(c4ccc5c(c4)sc4ccccc45)c4ccc(-n5c6ccccc6c6ccccc65)c5ccccc45)ccc-2c13 ; c1ccc(-c2ccc(N(c3ccc(-c4ccc(-n5c6ccccc6c6ccccc65)cc4)cc3)c3cccc([Si](c4ccccc4)(c4ccccc4)c4ccccc4)c3)cc2)cc1]

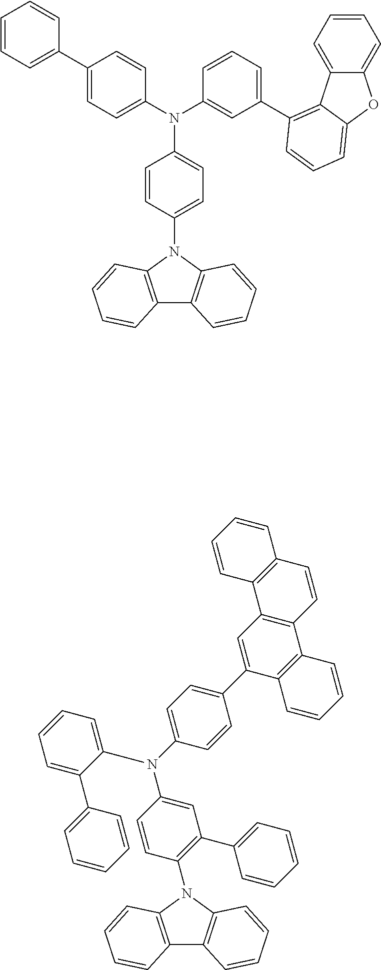 [SMILES: c1ccc(-c2ccc(N(c3ccc(-n4c5ccccc5c5ccccc54)cc3)c3cccc(-c4cccc5oc6ccccc6c45)c3)cc2)cc1 ; c1ccc(-c2ccccc2N(c2ccc(-c3cc4c5ccccc5ccc4c4ccccc34)cc2)c2ccc(-n3c4ccccc4c4ccccc43)c(-c3ccccc3)c2)cc1]

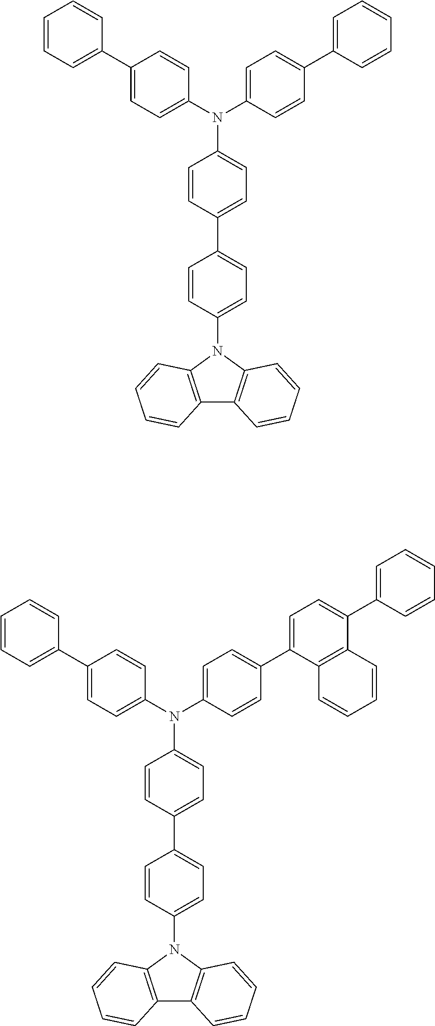 [SMILES: c1ccc(-c2ccc(N(c3ccc(-c4ccccc4)cc3)c3ccc(-c4ccc(-n5c6ccccc6c6ccccc65)cc4)cc3)cc2)cc1 ; c1ccc(-c2ccc(N(c3ccc(-c4ccc(-n5c6ccccc6c6ccccc65)cc4)cc3)c3ccc(-c4ccc(-c5ccccc5)c5ccccc45)cc3)cc2)cc1]

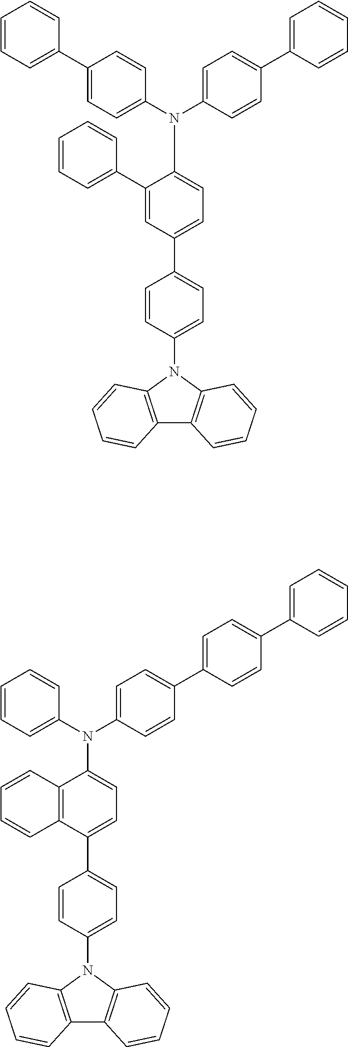 [SMILES: c1ccc(-c2ccc(N(c3ccc(-c4ccccc4)cc3)c3ccc(-c4ccc(-n5c6ccccc6c6ccccc65)cc4)cc3-c3ccccc3)cc2)cc1 ; c1ccc(-c2ccc(-c3ccc(N(c4ccccc4)c4ccc(-c5ccc(-n6c7ccccc7c7ccccc76)cc5)c5ccccc45)cc3)cc2)cc1]

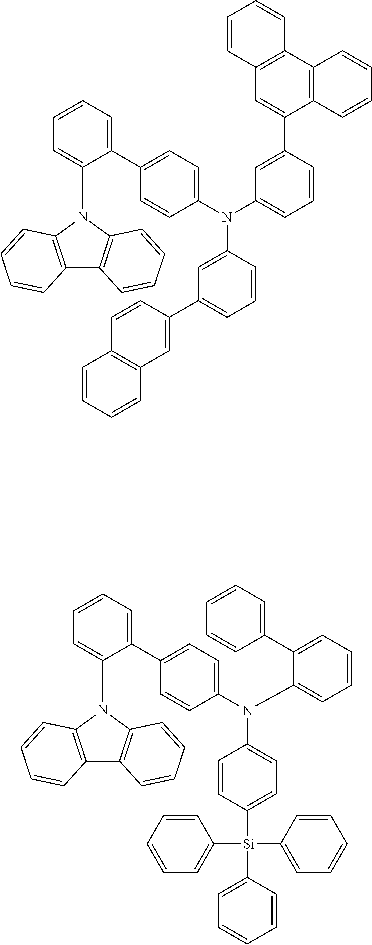 [SMILES: c1cc(-c2ccc3ccccc3c2)cc(N(c2ccc(-c3ccccc3-n3c4ccccc4c4ccccc43)cc2)c2cccc(-c3cc4ccccc4c4ccccc34)c2)c1 ; c1ccc(-c2ccccc2N(c2ccc(-c3ccccc3-n3c4ccccc4c4ccccc43)cc2)c2ccc([Si](c3ccccc3)(c3ccccc3)c3ccccc3)cc2)cc1]

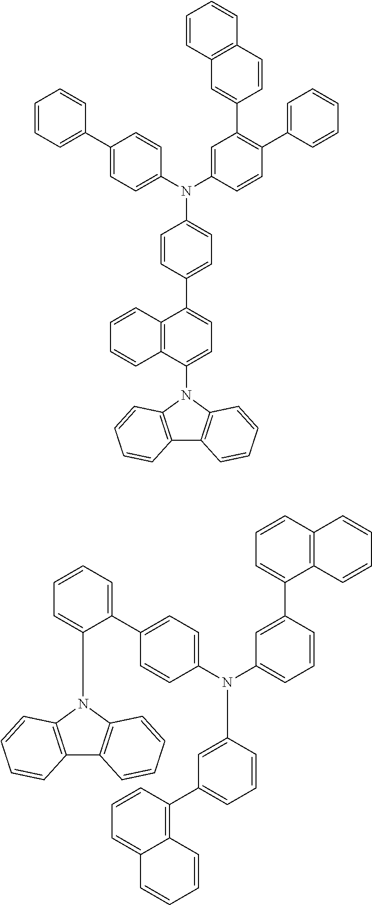 [SMILES: c1ccc(-c2ccc(N(c3ccc(-c4ccc(-n5c6ccccc6c6ccccc65)c5ccccc45)cc3)c3ccc(-c4ccccc4)c(-c4ccc5ccccc5c4)c3)cc2)cc1 ; c1cc(-c2cccc3ccccc23)cc(N(c2ccc(-c3ccccc3-n3c4ccccc4c4ccccc43)cc2)c2cccc(-c3cccc4ccccc34)c2)c1]

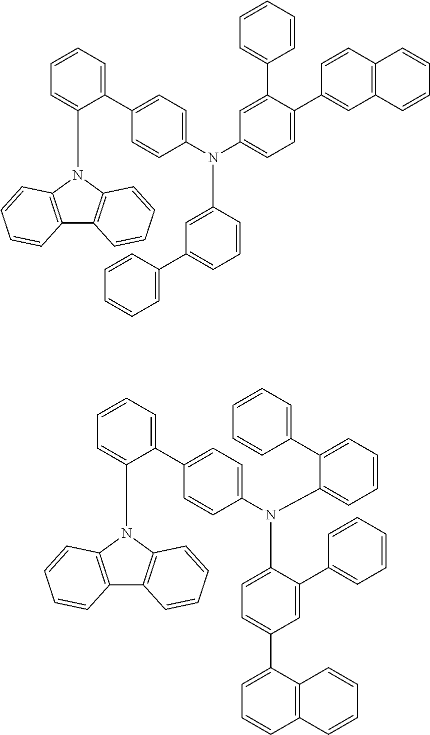 [SMILES: c1ccc(-c2cccc(N(c3ccc(-c4ccccc4-n4c5ccccc5c5ccccc54)cc3)c3ccc(-c4ccc5ccccc5c4)c(-c4ccccc4)c3)c2)cc1 ; c1ccc(-c2ccccc2N(c2ccc(-c3ccccc3-n3c4ccccc4c4ccccc43)cc2)c2ccc(-c3cccc4ccccc34)cc2-c2ccccc2)cc1]

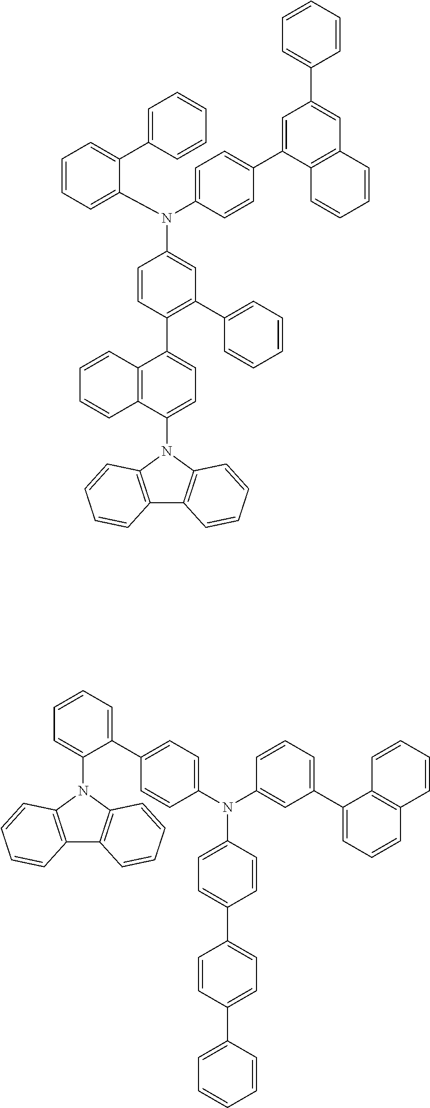 [SMILES: c1ccc(-c2cc(-c3ccc(N(c4ccc(-c5ccc(-n6c7ccccc7c7ccccc76)c6ccccc56)c(-c5ccccc5)c4)c4ccccc4-c4ccccc4)cc3)c3ccccc3c2)cc1 ; c1ccc(-c2ccc(-c3ccc(N(c4ccc(-c5ccccc5-n5c6ccccc6c6ccccc65)cc4)c4cccc(-c5cccc6ccccc56)c4)cc3)cc2)cc1]

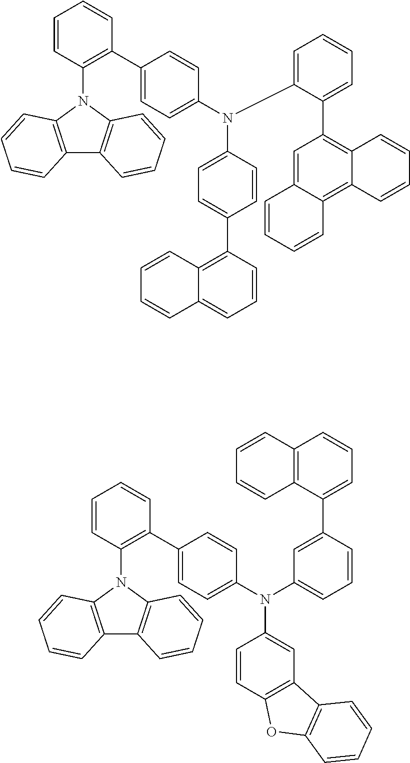 [SMILES: c1ccc(N(c2ccc(-c3ccccc3-n3c4ccccc4c4ccccc43)cc2)c2ccc(-c3cccc4ccccc34)cc2)c(-c2cc3ccccc3c3ccccc23)c1 ; c1cc(-c2cccc3ccccc23)cc(N(c2ccc(-c3ccccc3-n3c4ccccc4c4ccccc43)cc2)c2ccc3oc4ccccc4c3c2)c1]

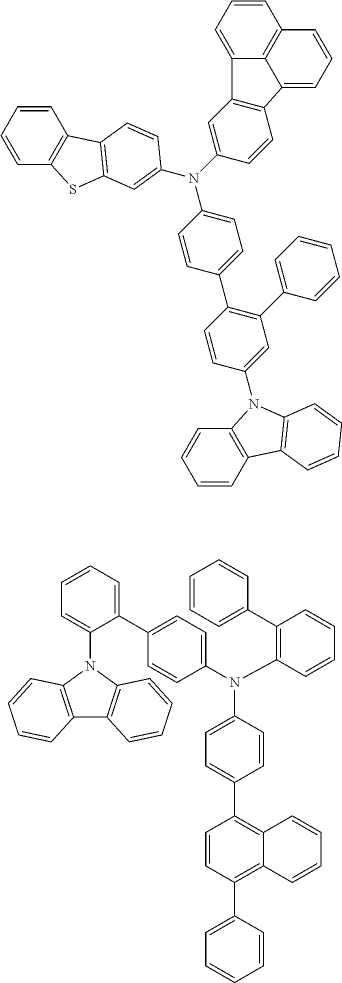 [SMILES: c1ccc(-c2cc(-n3c4ccccc4c4ccccc43)ccc2-c2ccc(N(c3ccc4c(c3)-c3cccc5cccc-4c35)c3ccc4c(c3)sc3ccccc34)cc2)cc1 ; c1ccc(-c2ccccc2N(c2ccc(-c3ccccc3-n3c4ccccc4c4ccccc43)cc2)c2ccc(-c3ccc(-c4ccccc4)c4ccccc34)cc2)cc1]

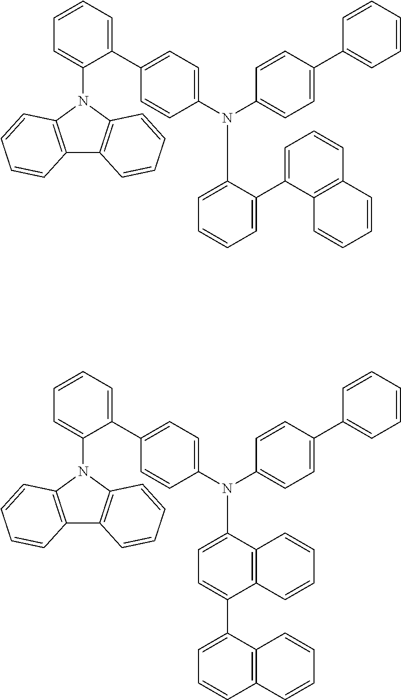 [SMILES: c1ccc(-c2ccc(N(c3ccc(-c4ccccc4-n4c5ccccc5c5ccccc54)cc3)c3ccccc3-c3cccc4ccccc34)cc2)cc1 ; c1ccc(-c2ccc(N(c3ccc(-c4ccccc4-n4c5ccccc5c5ccccc54)cc3)c3ccc(-c4cccc5ccccc45)c4ccccc34)cc2)cc1]

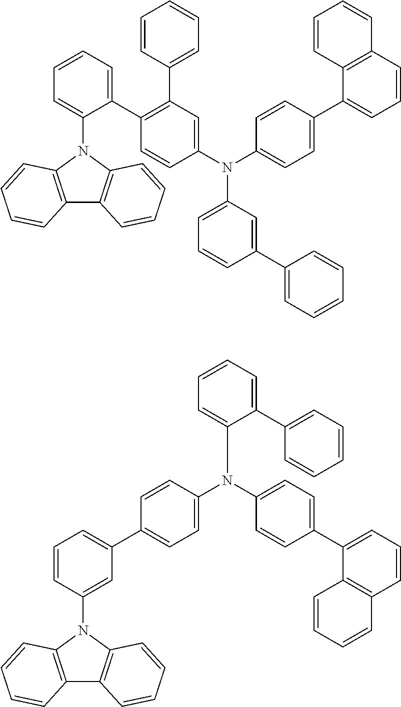 [SMILES: c1ccc(-c2cccc(N(c3ccc(-c4cccc5ccccc45)cc3)c3ccc(-c4ccccc4-n4c5ccccc5c5ccccc54)c(-c4ccccc4)c3)c2)cc1 ; c1ccc(-c2ccccc2N(c2ccc(-c3cccc(-n4c5ccccc5c5ccccc54)c3)cc2)c2ccc(-c3cccc4ccccc34)cc2)cc1]

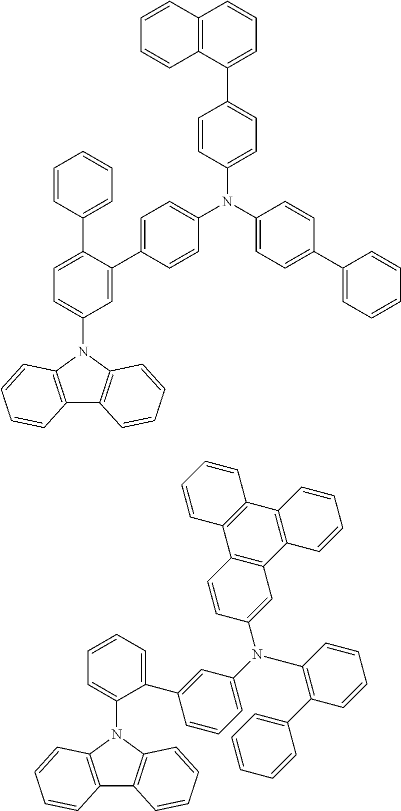 [SMILES: c1ccc(-c2ccc(N(c3ccc(-c4cc(-n5c6ccccc6c6ccccc65)ccc4-c4ccccc4)cc3)c3ccc(-c4cccc5ccccc45)cc3)cc2)cc1 ; c1ccc(-c2ccccc2N(c2cccc(-c3ccccc3-n3c4ccccc4c4ccccc43)c2)c2ccc3c4ccccc4c4ccccc4c3c2)cc1]

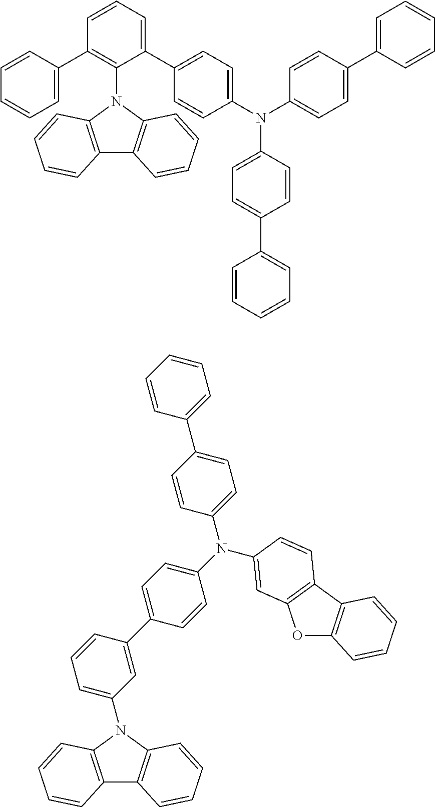 [SMILES: c1ccc(-c2ccc(N(c3ccc(-c4ccccc4)cc3)c3ccc(-c4cccc(-c5ccccc5)c4-n4c5ccccc5c5ccccc54)cc3)cc2)cc1 ; c1ccc(-c2ccc(N(c3ccc(-c4cccc(-n5c6ccccc6c6ccccc65)c4)cc3)c3ccc4c(c3)oc3ccccc34)cc2)cc1]

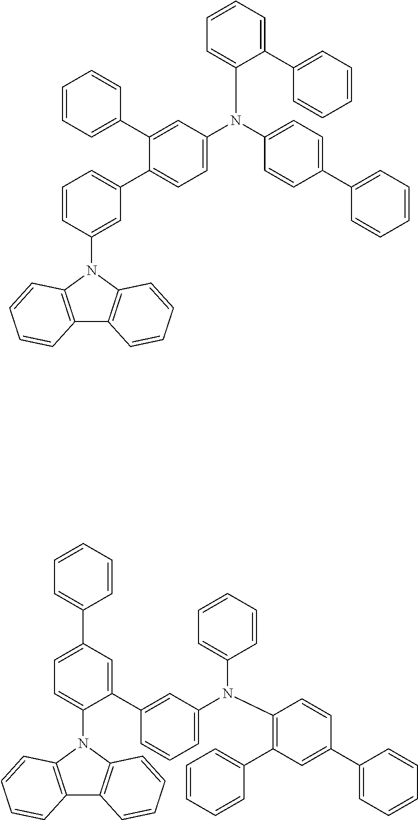 [SMILES: c1ccc(-c2ccc(N(c3ccc(-c4cccc(-n5c6ccccc6c6ccccc65)c4)c(-c4ccccc4)c3)c3ccccc3-c3ccccc3)cc2)cc1 ; c1ccc(-c2ccc(N(c3ccccc3)c3cccc(-c4cc(-c5ccccc5)ccc4-n4c5ccccc5c5ccccc54)c3)c(-c3ccccc3)c2)cc1]

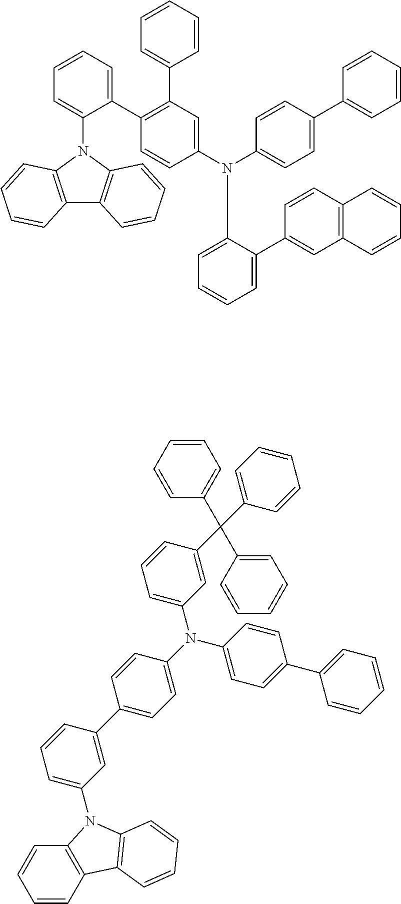 [SMILES: c1ccc(-c2ccc(N(c3ccc(-c4ccccc4-n4c5ccccc5c5ccccc54)c(-c4ccccc4)c3)c3ccccc3-c3ccc4ccccc4c3)cc2)cc1 ; c1ccc(-c2ccc(N(c3ccc(-c4cccc(-n5c6ccccc6c6ccccc65)c4)cc3)c3cccc(C(c4ccccc4)(c4ccccc4)c4ccccc4)c3)cc2)cc1]

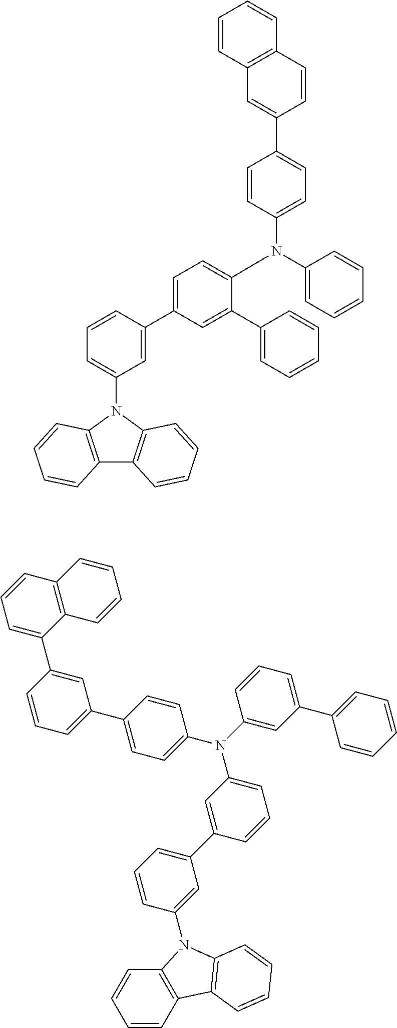 [SMILES: c1ccc(-c2cc(-c3cccc(-n4c5ccccc5c5ccccc54)c3)ccc2N(c2ccccc2)c2ccc(-c3ccc4ccccc4c3)cc2)cc1 ; c1ccc(-c2cccc(N(c3ccc(-c4cccc(-c5cccc6ccccc56)c4)cc3)c3cccc(-c4cccc(-n5c6ccccc6c6ccccc65)c4)c3)c2)cc1]

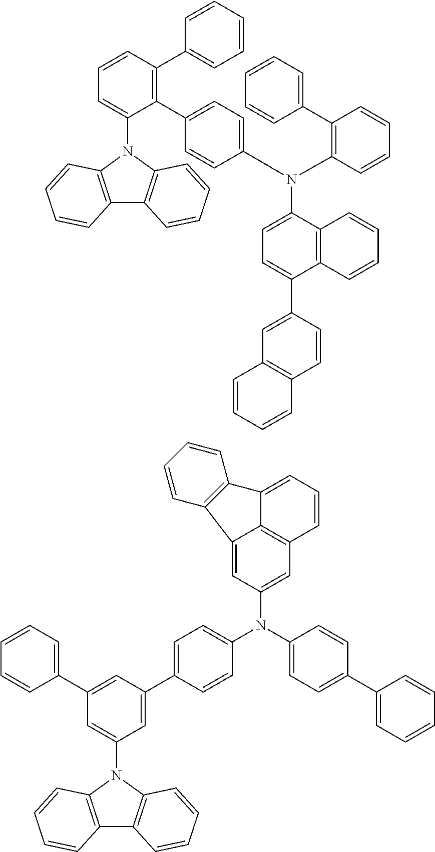

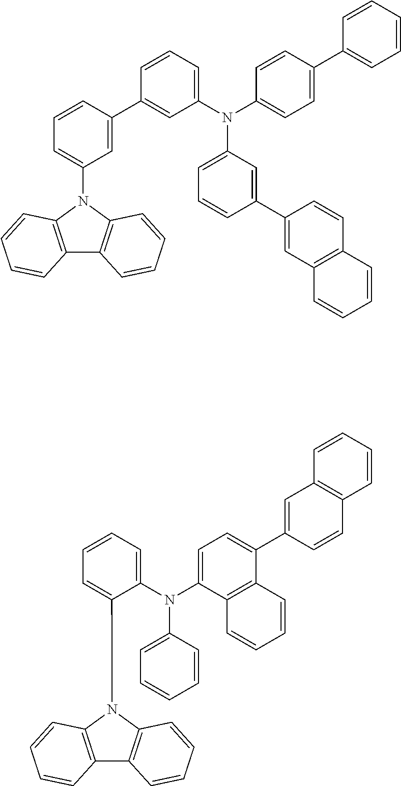 [SMILES: c1ccc(-c2ccc(N(c3cccc(-c4cccc(-n5c6ccccc6c6ccccc65)c4)c3)c3cccc(-c4ccc5ccccc5c4)c3)cc2)cc1 ; c1ccc(N(c2ccccc2-n2c3ccccc3c3ccccc32)c2ccc(-c3ccc4ccccc4c3)c3ccccc23)cc1]

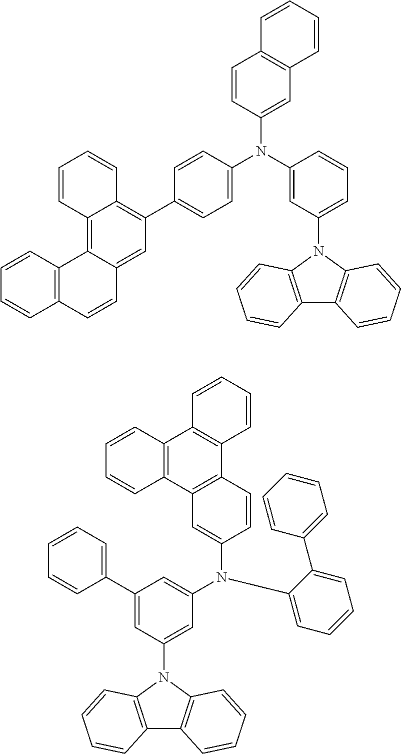 [SMILES: c1cc(N(c2ccc(-c3cc4ccc5ccccc5c4c4ccccc34)cc2)c2ccc3ccccc3c2)cc(-n2c3ccccc3c3ccccc32)c1 ; c1ccc(-c2cc(N(c3ccc4c5ccccc5c5ccccc5c4c3)c3ccccc3-c3ccccc3)cc(-n3c4ccccc4c4ccccc43)c2)cc1]

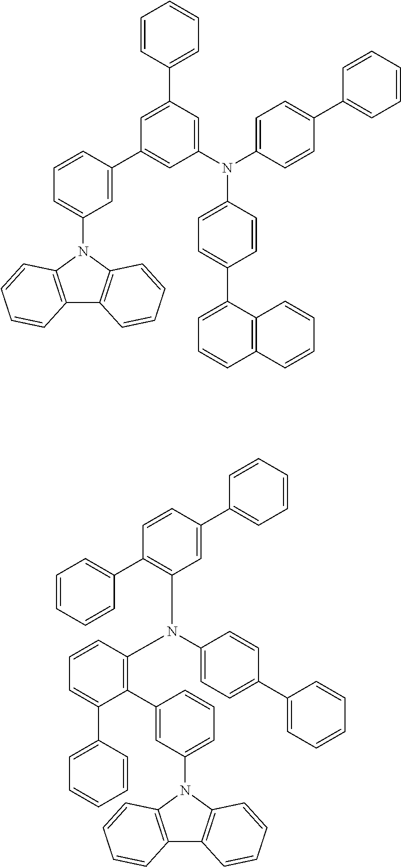 [SMILES: c1ccc(-c2ccc(N(c3ccc(-c4cccc5ccccc45)cc3)c3cc(-c4ccccc4)cc(-c4cccc(-n5c6ccccc6c6ccccc65)c4)c3)cc2)cc1 ; c1ccc(-c2ccc(N(c3cc(-c4ccccc4)ccc3-c3ccccc3)c3cccc(-c4ccccc4)c3-c3cccc(-n4c5ccccc5c5ccccc54)c3)cc2)cc1]

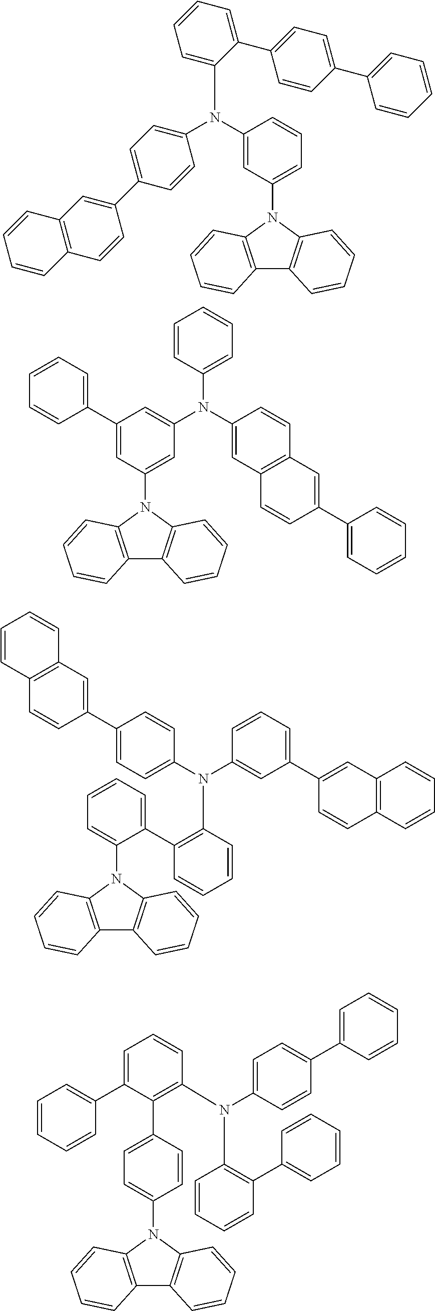

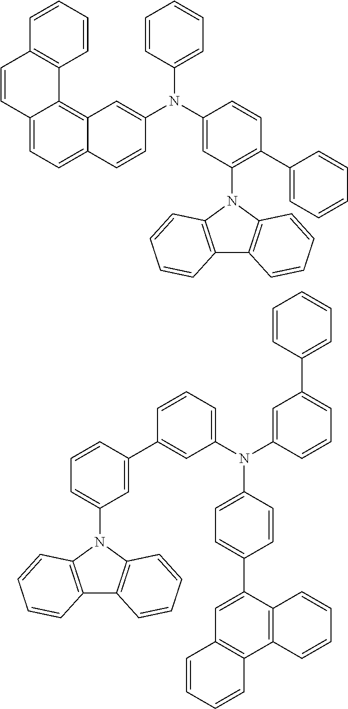

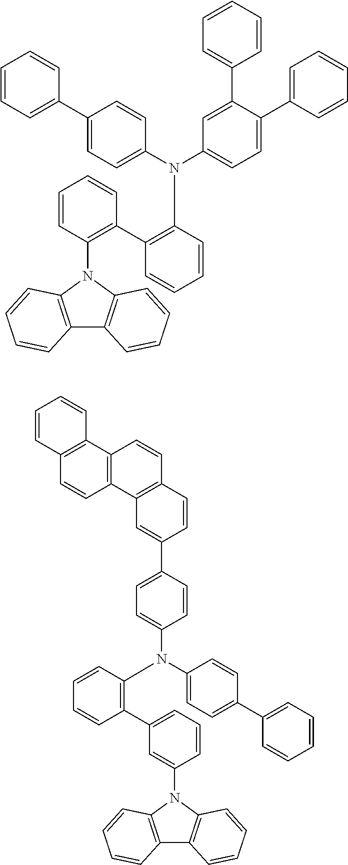 [SMILES: c1ccc(-c2ccc(N(c3ccc(-c4ccccc4)c(-c4ccccc4)c3)c3ccccc3-c3ccccc3-n3c4ccccc4c4ccccc43)cc2)cc1 ; c1ccc(-c2ccc(N(c3ccc(-c4ccc5ccc6c7ccccc7ccc6c5c4)cc3)c3ccccc3-c3cccc(-n4c5ccccc5c5ccccc54)c3)cc2)cc1]

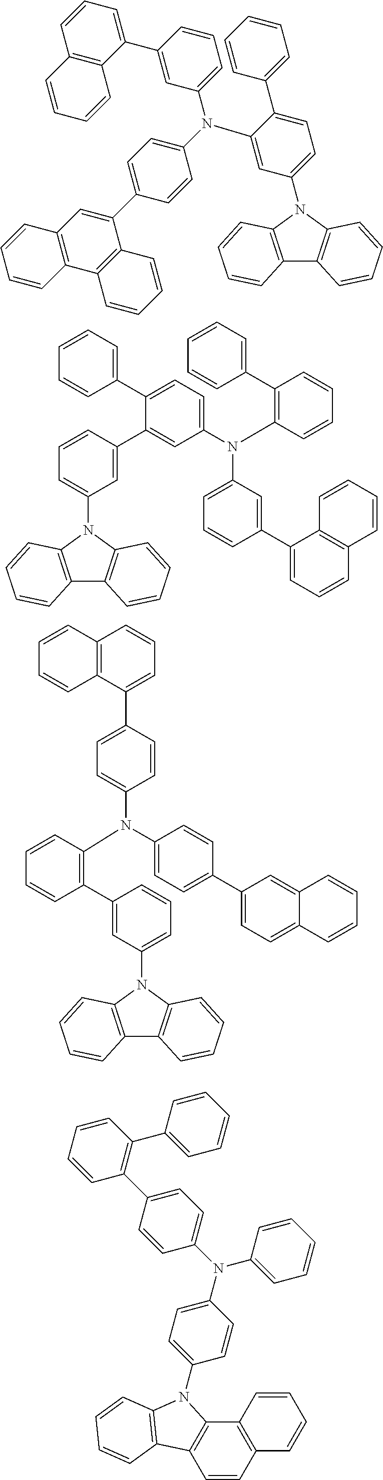

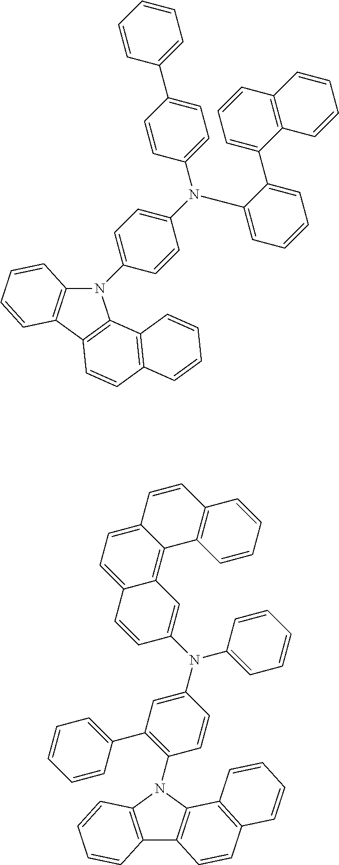 [SMILES: c1ccc(-c2ccc(N(c3ccc(-n4c5ccccc5c5ccc6ccccc6c54)cc3)c3ccccc3-c3cccc4ccccc34)cc2)cc1 ; c1ccc(-c2cc(N(c3ccccc3)c3ccc4ccc5ccc6ccccc6c5c4c3)ccc2-n2c3ccccc3c3ccc4ccccc4c32)cc1]

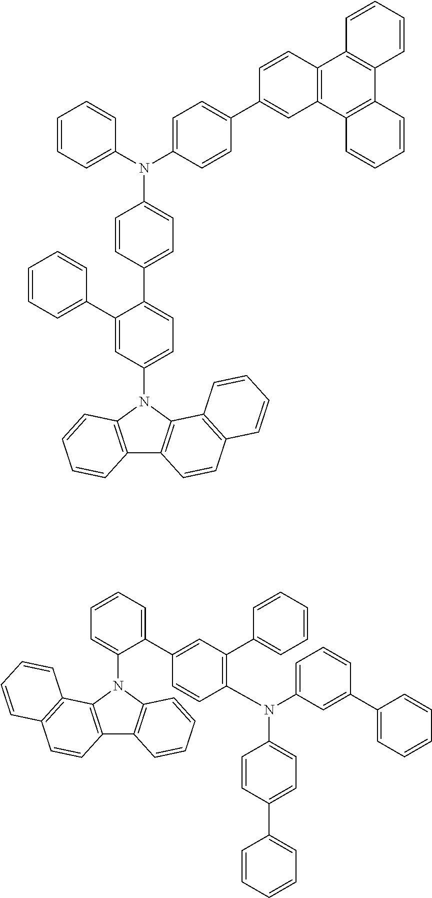 [SMILES: c1ccc(-c2cc(-n3c4ccccc4c4ccc5ccccc5c43)ccc2-c2ccc(N(c3ccccc3)c3ccc(-c4ccc5c6ccccc6c6ccccc6c5c4)cc3)cc2)cc1 ; c1ccc(-c2ccc(N(c3cccc(-c4ccccc4)c3)c3ccc(-c4ccccc4-n4c5ccccc5c5ccc6ccccc6c54)cc3-c3ccccc3)cc2)cc1]

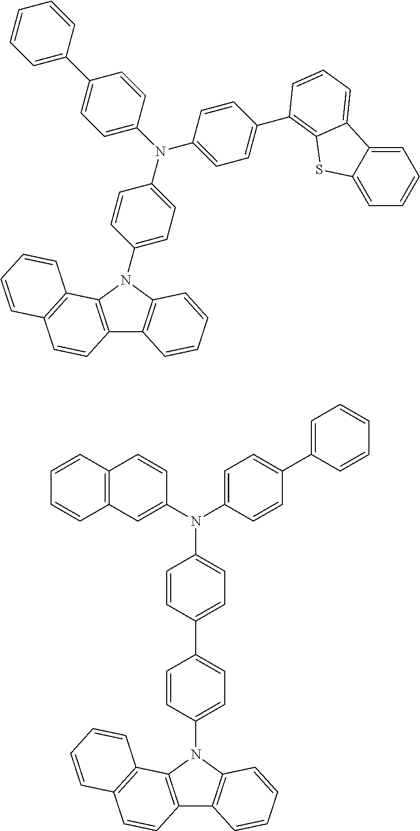 [SMILES: c1ccc(-c2ccc(N(c3ccc(-c4cccc5c4sc4ccccc45)cc3)c3ccc(-n4c5ccccc5c5ccc6ccccc6c54)cc3)cc2)cc1 ; c1ccc(-c2ccc(N(c3ccc(-c4ccc(-n5c6ccccc6c6ccc7ccccc7c65)cc4)cc3)c3ccc4ccccc4c3)cc2)cc1]

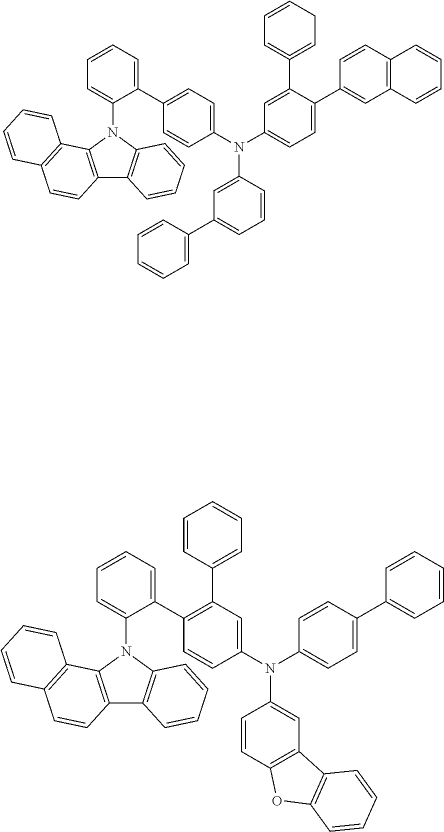

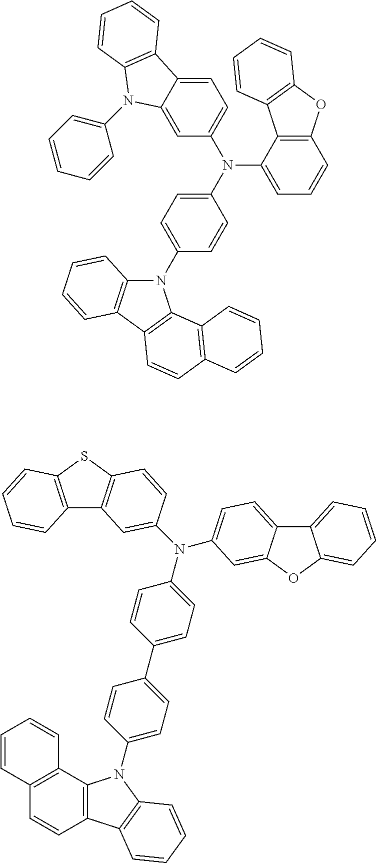 [SMILES: c1ccc(-n2c3ccccc3c3ccc(N(c4ccc(-n5c6ccccc6c6ccc7ccccc7c65)cc4)c4cccc5oc6ccccc6c45)cc32)cc1 ; c1ccc2c(c1)ccc1c3ccccc3n(-c3ccc(-c4ccc(N(c5ccc6c(c5)oc5ccccc56)c5ccc6sc7ccccc7c6c5)cc4)cc3)c21]

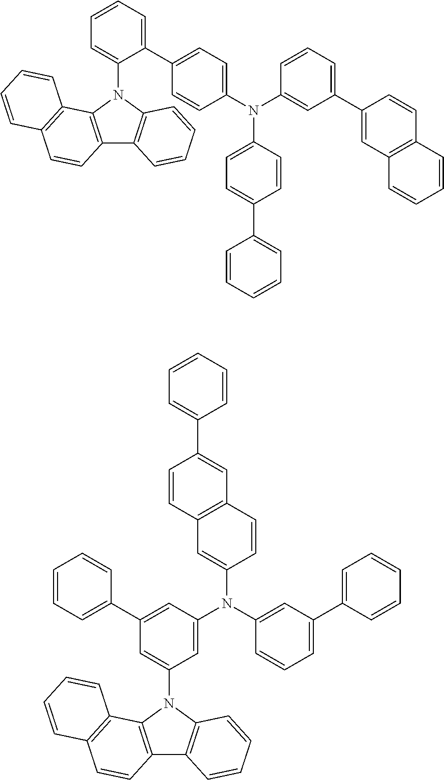 [SMILES: c1ccc(-c2ccc(N(c3ccc(-c4ccccc4-n4c5ccccc5c5ccc6ccccc6c54)cc3)c3cccc(-c4ccc5ccccc5c4)c3)cc2)cc1 ; c1ccc(-c2cccc(N(c3cc(-c4ccccc4)cc(-n4c5ccccc5c5ccc6ccccc6c54)c3)c3ccc4cc(-c5ccccc5)ccc4c3)c2)cc1]

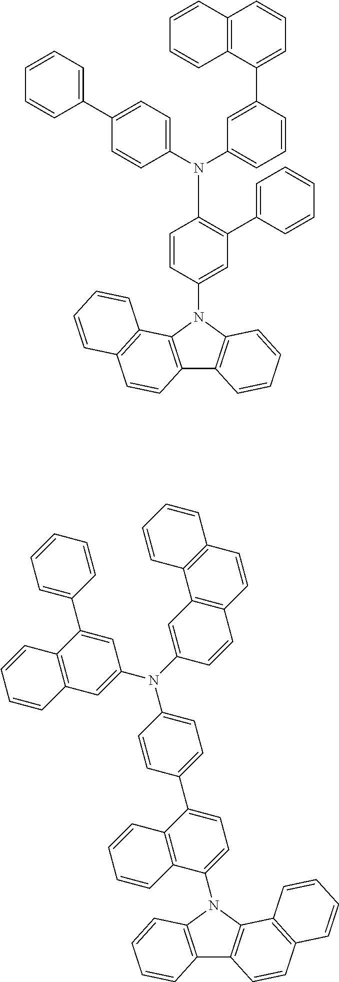 [SMILES: c1ccc(-c2ccc(N(c3cccc(-c4cccc5ccccc45)c3)c3ccc(-n4c5ccccc5c5ccc6ccccc6c54)cc3-c3ccccc3)cc2)cc1 ; c1ccc(-c2cc(N(c3ccc(-c4ccc(-n5c6ccccc6c6ccc7ccccc7c65)c5ccccc45)cc3)c3ccc4ccc5ccccc5c4c3)cc3ccccc23)cc1]

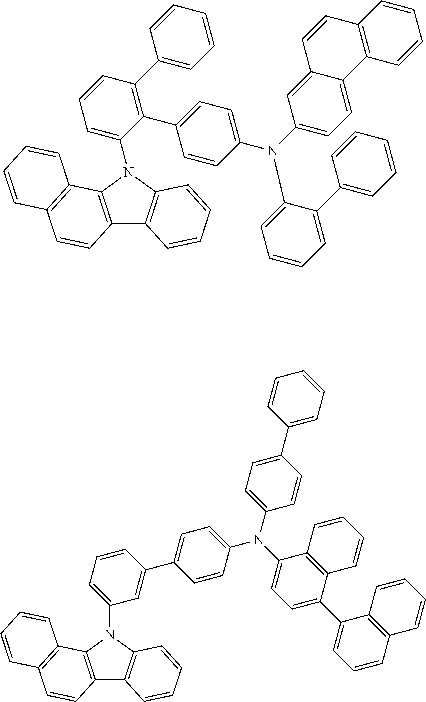 [SMILES: c1ccc(-c2ccccc2N(c2ccc(-c3c(-c4ccccc4)cccc3-n3c4ccccc4c4ccc5ccccc5c43)cc2)c2ccc3c(ccc4ccccc43)c2)cc1 ; c1ccc(-c2ccc(N(c3ccc(-c4cccc(-n5c6ccccc6c6ccc7ccccc7c65)c4)cc3)c3ccc(-c4cccc5ccccc45)c4ccccc34)cc2)cc1]

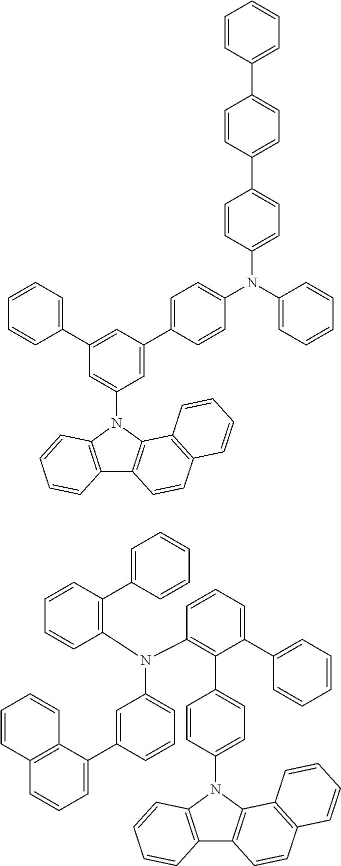

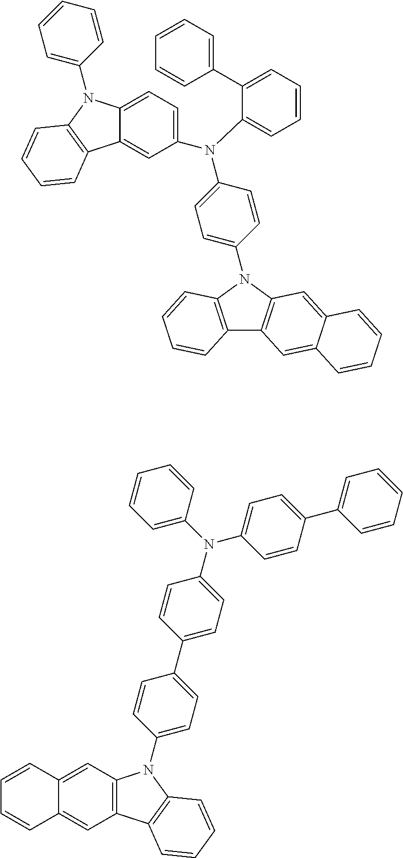 [SMILES: c1ccc(-c2ccccc2N(c2ccc(-n3c4ccccc4c4cc5ccccc5cc43)cc2)c2ccc3c(c2)c2ccccc2n3-c2ccccc2)cc1 ; c1ccc(-c2ccc(N(c3ccccc3)c3ccc(-c4ccc(-n5c6ccccc6c6cc7ccccc7cc65)cc4)cc3)cc2)cc1]

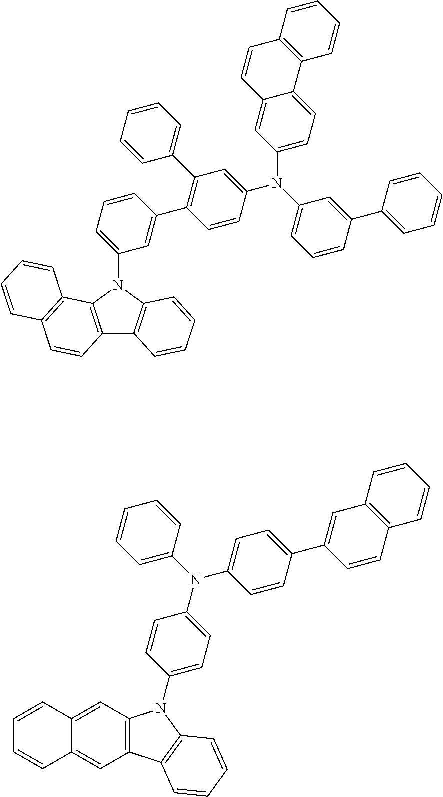 [SMILES: c1ccc(-c2cccc(N(c3ccc(-c4cccc(-n5c6ccccc6c6ccc7ccccc7c65)c4)c(-c4ccccc4)c3)c3ccc4c(ccc5ccccc54)c3)c2)cc1 ; c1ccc(N(c2ccc(-c3ccc4ccccc4c3)cc2)c2ccc(-n3c4ccccc4c4cc5ccccc5cc43)cc2)cc1]

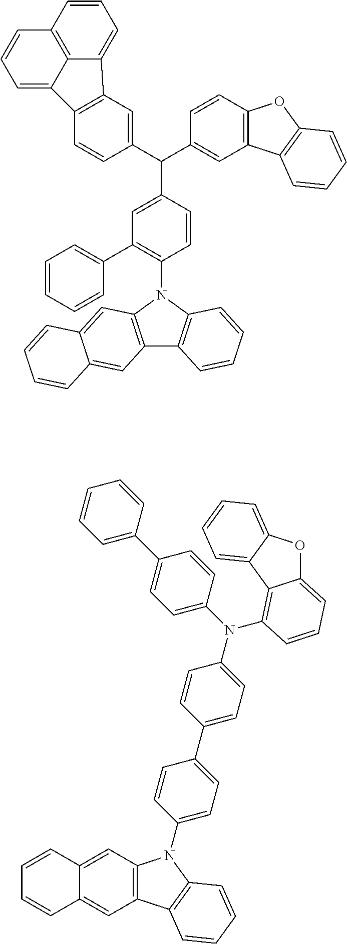 [SMILES: c1ccc(-c2cc(C(c3ccc4c(c3)-c3cccc5cccc-4c35)c3ccc4oc5ccccc5c4c3)ccc2-n2c3ccccc3c3cc4ccccc4cc32)cc1 ; c1ccc(-c2ccc(N(c3ccc(-c4ccc(-n5c6ccccc6c6cc7ccccc7cc65)cc4)cc3)c3cccc4oc5ccccc5c34)cc2)cc1]

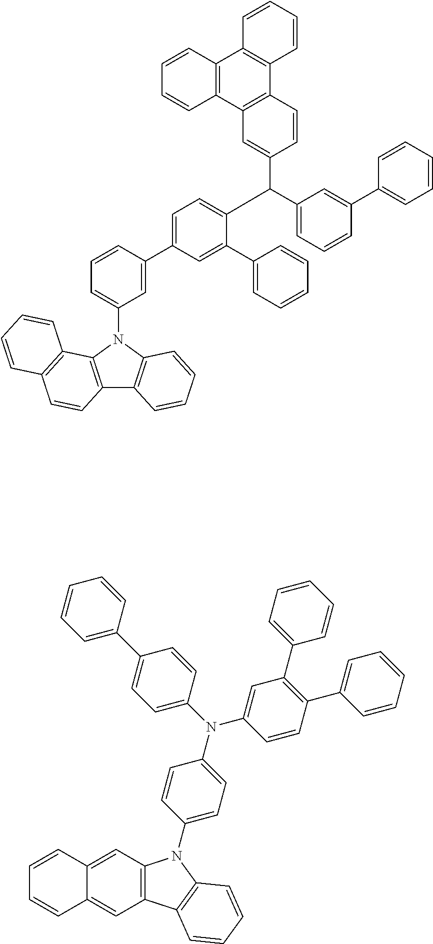 [SMILES: c1ccc(-c2cccc(C(c3ccc4c5ccccc5c5ccccc5c4c3)c3ccc(-c4cccc(-n5c6ccccc6c6ccc7ccccc7c65)c4)cc3-c3ccccc3)c2)cc1 ; c1ccc(-c2ccc(N(c3ccc(-n4c5ccccc5c5cc6ccccc6cc54)cc3)c3ccc(-c4ccccc4)c(-c4ccccc4)c3)cc2)cc1]

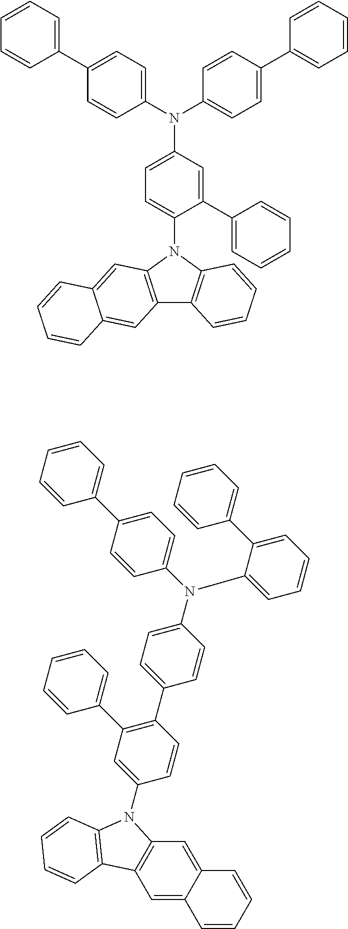 [SMILES: c1ccc(-c2ccc(N(c3ccc(-c4ccccc4)cc3)c3ccc(-n4c5ccccc5c5cc6ccccc6cc54)c(-c4ccccc4)c3)cc2)cc1 ; c1ccc(-c2ccc(N(c3ccc(-c4ccc(-n5c6ccccc6c6cc7ccccc7cc65)cc4-c4ccccc4)cc3)c3ccccc3-c3ccccc3)cc2)cc1]

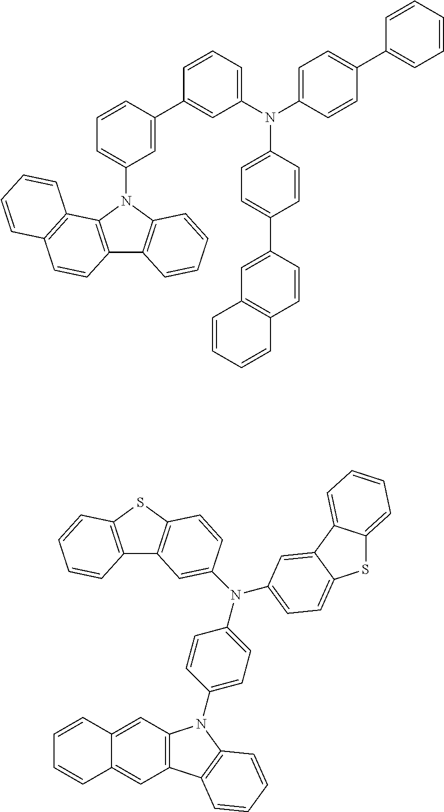 [SMILES: c1ccc(-c2ccc(N(c3ccc(-c4ccc5ccccc5c4)cc3)c3cccc(-c4cccc(-n5c6ccccc6c6ccc7ccccc7c65)c4)c3)cc2)cc1 ; c1ccc2cc3c(cc2c1)c1ccccc1n3-c1ccc(N(c2ccc3sc4ccccc4c3c2)c2ccc3sc4ccccc4c3c2)cc1]

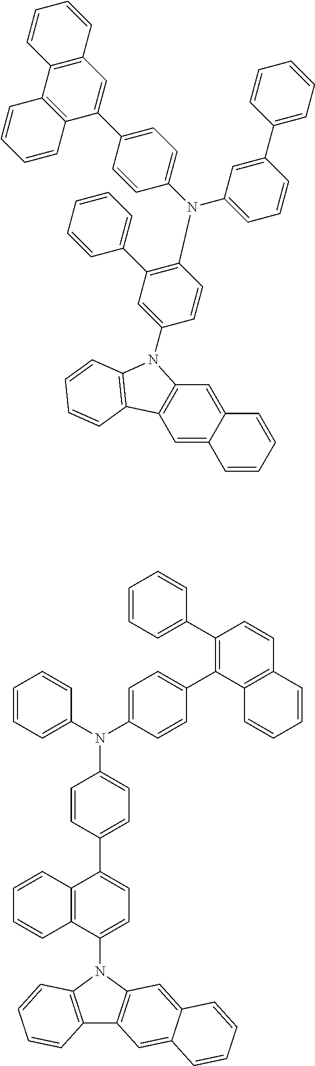 [SMILES: c1ccc(-c2cccc(N(c3ccc(-c4cc5ccccc5c5ccccc45)cc3)c3ccc(-n4c5ccccc5c5cc6ccccc6cc54)cc3-c3ccccc3)c2)cc1 ; c1ccc(-c2ccc3ccccc3c2-c2ccc(N(c3ccccc3)c3ccc(-c4ccc(-n5c6ccccc6c6cc7ccccc7cc65)c5ccccc45)cc3)cc2)cc1]

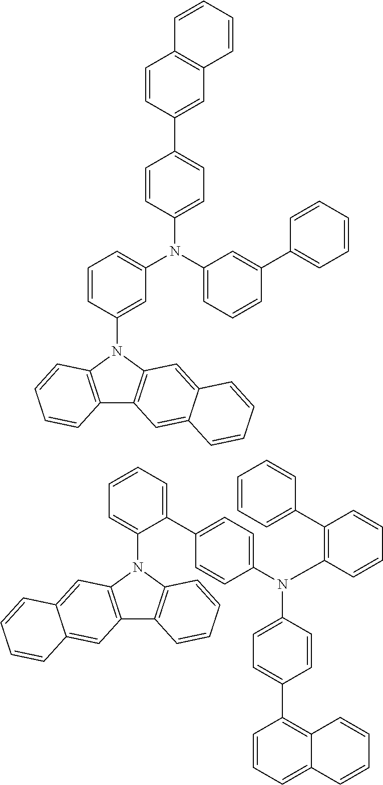

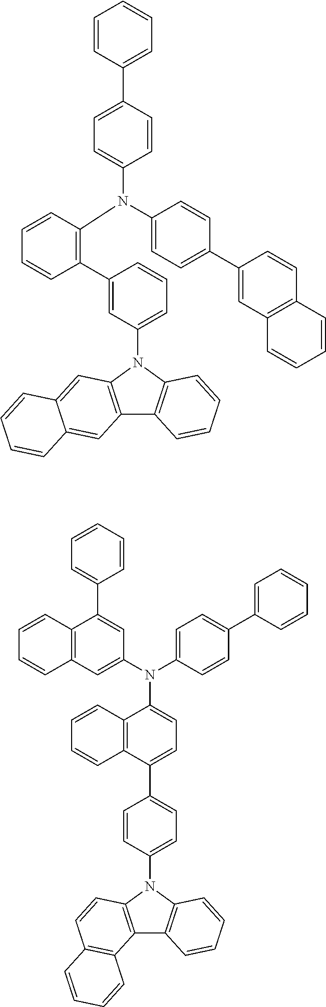 [SMILES: c1ccc(-c2ccc(N(c3ccc(-c4ccc5ccccc5c4)cc3)c3ccccc3-c3cccc(-n4c5ccccc5c5cc6ccccc6cc54)c3)cc2)cc1 ; c1ccc(-c2ccc(N(c3cc(-c4ccccc4)c4ccccc4c3)c3ccc(-c4ccc(-n5c6ccccc6c6c7ccccc7ccc65)cc4)c4ccccc34)cc2)cc1]

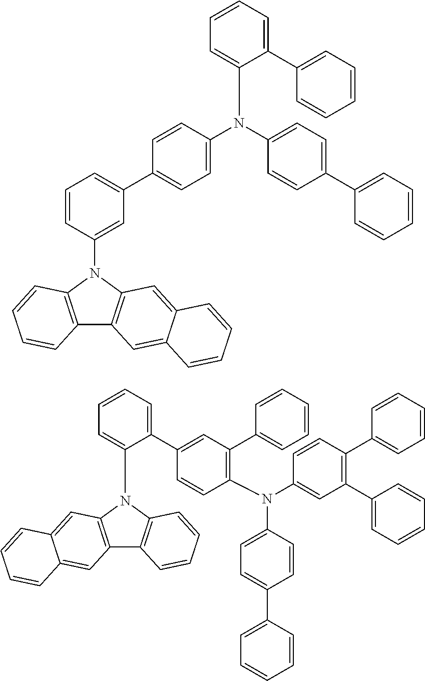

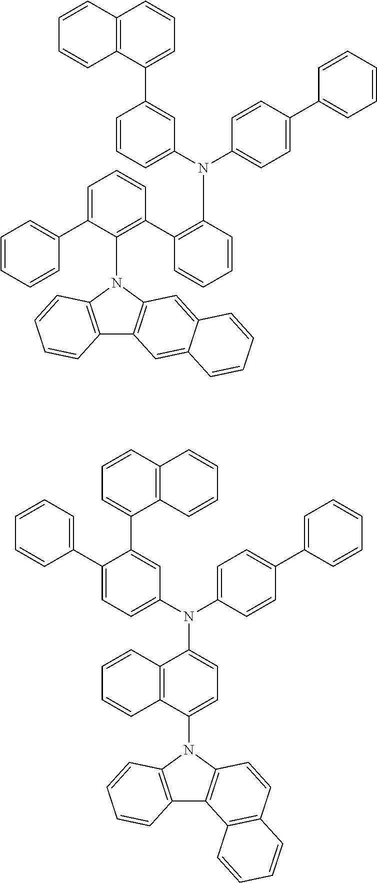 [SMILES: c1ccc(-c2ccc(N(c3cccc(-c4cccc5ccccc45)c3)c3ccccc3-c3cccc(-c4ccccc4)c3-n3c4ccccc4c4cc5ccccc5cc43)cc2)cc1 ; c1ccc(-c2ccc(N(c3ccc(-c4ccccc4)c(-c4cccc5ccccc45)c3)c3ccc(-n4c5ccccc5c5c6ccccc6ccc54)c4ccccc34)cc2)cc1]

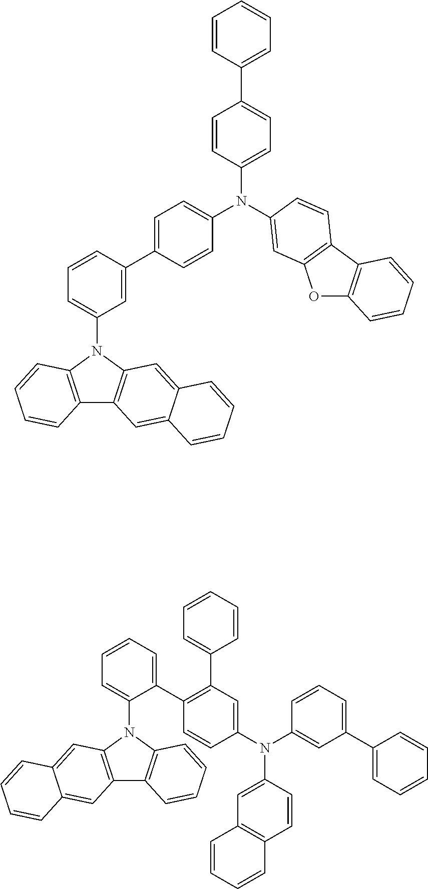 [SMILES: c1ccc(-c2ccc(N(c3ccc(-c4cccc(-n5c6ccccc6c6cc7ccccc7cc65)c4)cc3)c3ccc4c(c3)oc3ccccc34)cc2)cc1 ; c1ccc(-c2cccc(N(c3ccc(-c4ccccc4-n4c5ccccc5c5cc6ccccc6cc54)c(-c4ccccc4)c3)c3ccc4ccccc4c3)c2)cc1]

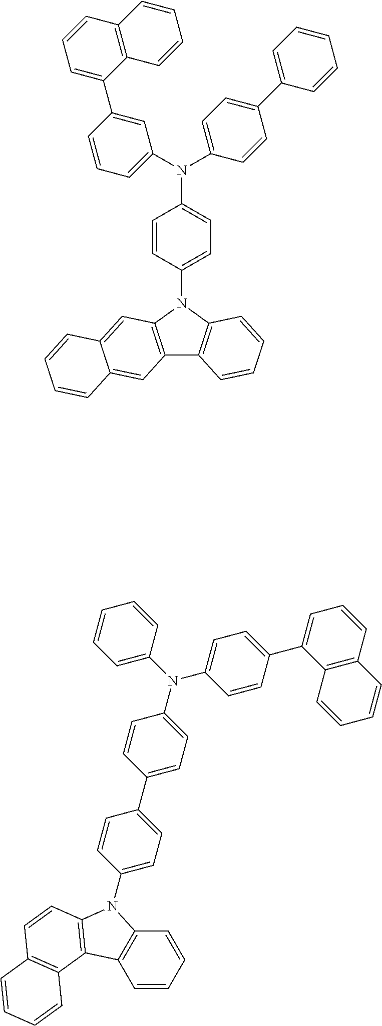 [SMILES: c1ccc(-c2ccc(N(c3ccc(-n4c5ccccc5c5cc6ccccc6cc54)cc3)c3cccc(-c4cccc5ccccc45)c3)cc2)cc1 ; c1ccc(N(c2ccc(-c3ccc(-n4c5ccccc5c5c6ccccc6ccc54)cc3)cc2)c2ccc(-c3cccc4ccccc34)cc2)cc1]

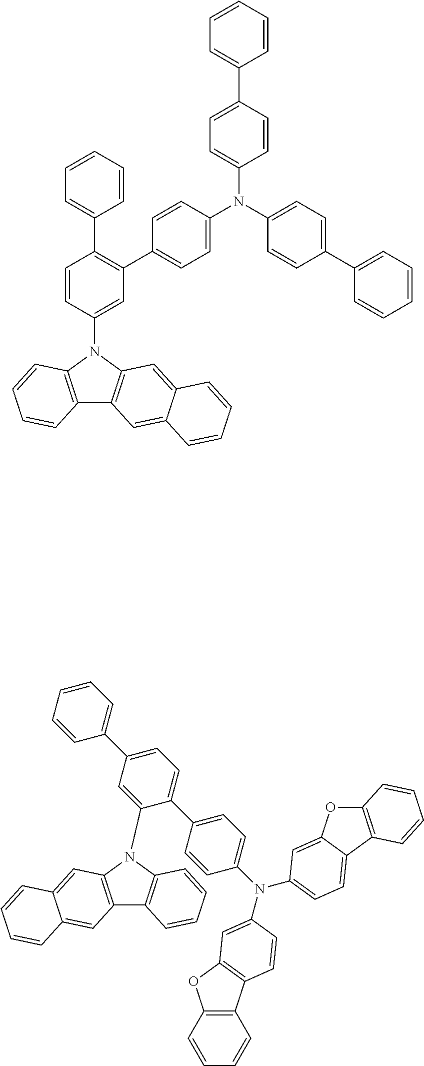 [SMILES: c1ccc(-c2ccc(N(c3ccc(-c4ccccc4)cc3)c3ccc(-c4cc(-n5c6ccccc6c6cc7ccccc7cc65)ccc4-c4ccccc4)cc3)cc2)cc1 ; c1ccc(-c2ccc(-c3ccc(N(c4ccc5c(c4)oc4ccccc45)c4ccc5c(c4)oc4ccccc45)cc3)c(-n3c4ccccc4c4cc5ccccc5cc43)c2)cc1]

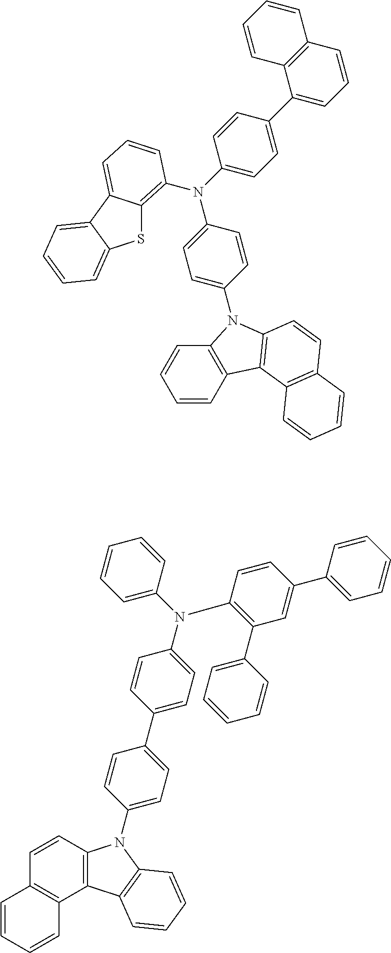 [SMILES: c1ccc2c(-c3ccc(N(c4ccc(-n5c6ccccc6c6c7ccccc7ccc65)cc4)c4cccc5c4sc4ccccc45)cc3)cccc2c1 ; c1ccc(-c2ccc(N(c3ccccc3)c3ccc(-c4ccc(-n5c6ccccc6c6c7ccccc7ccc65)cc4)cc3)c(-c3ccccc3)c2)cc1]

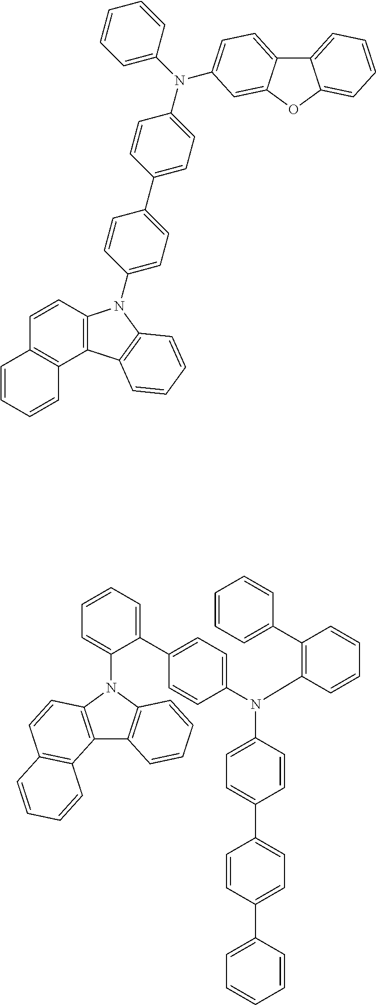 [SMILES: c1ccc(N(c2ccc(-c3ccc(-n4c5ccccc5c5c6ccccc6ccc54)cc3)cc2)c2ccc3c(c2)oc2ccccc23)cc1 ; c1ccc(-c2ccc(-c3ccc(N(c4ccc(-c5ccccc5-n5c6ccccc6c6c7ccccc7ccc65)cc4)c4ccccc4-c4ccccc4)cc3)cc2)cc1]

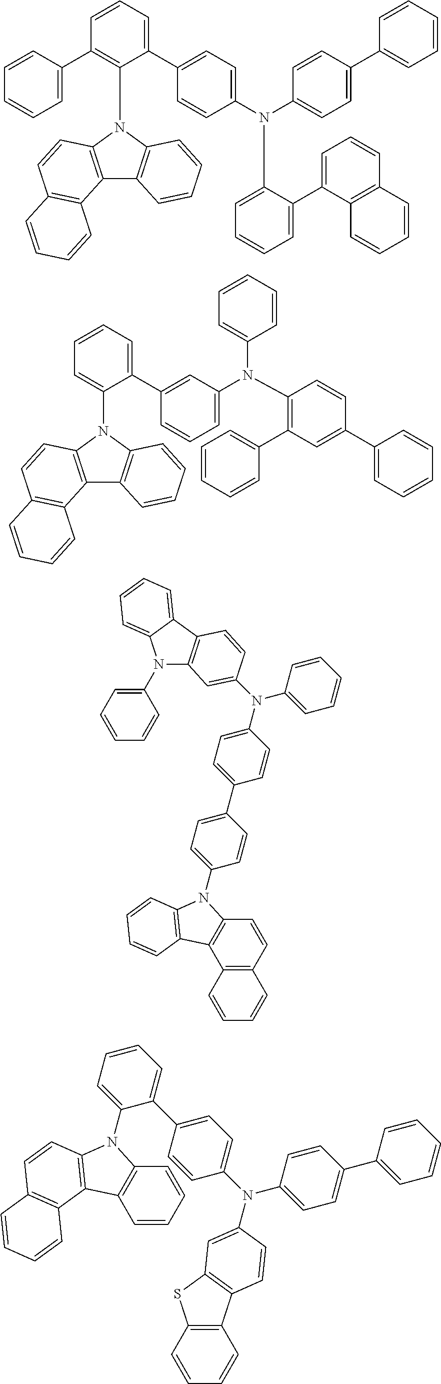

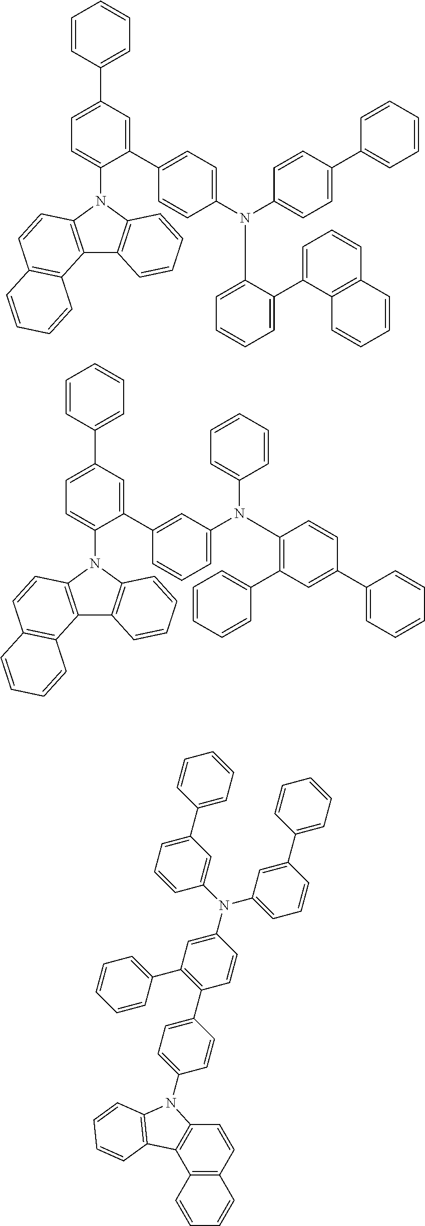 [SMILES: c1ccc(-c2ccc(N(c3ccc(-c4cc(-c5ccccc5)ccc4-n4c5ccccc5c5c6ccccc6ccc54)cc3)c3ccccc3-c3cccc4ccccc34)cc2)cc1 ; c1ccc(-c2ccc(N(c3ccccc3)c3cccc(-c4cc(-c5ccccc5)ccc4-n4c5ccccc5c5c6ccccc6ccc54)c3)c(-c3ccccc3)c2)cc1 ; c1ccc(-c2cccc(N(c3cccc(-c4ccccc4)c3)c3ccc(-c4ccc(-n5c6ccccc6c6c7ccccc7ccc65)cc4)c(-c4ccccc4)c3)c2)cc1]

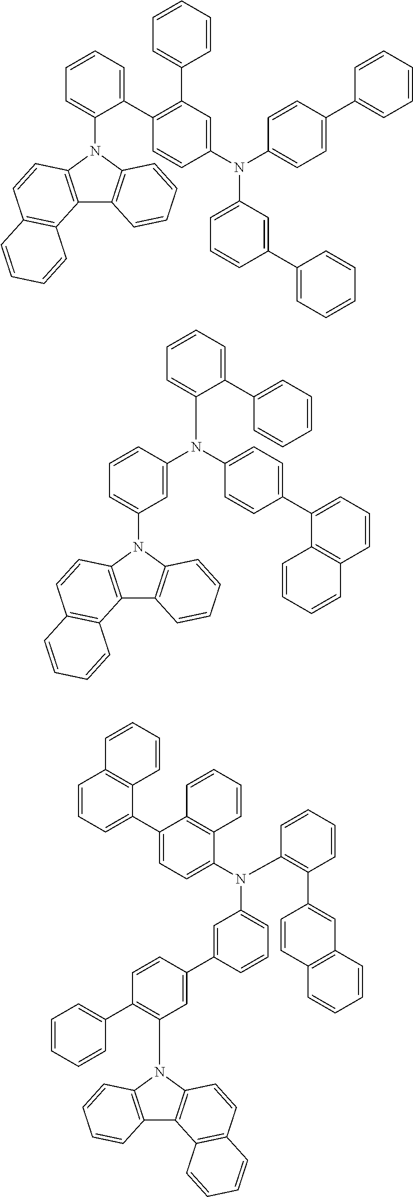 [SMILES: c1ccc(-c2ccc(N(c3cccc(-c4ccccc4)c3)c3ccc(-c4ccccc4-n4c5ccccc5c5c6ccccc6ccc54)c(-c4ccccc4)c3)cc2)cc1 ; c1ccc(-c2ccccc2N(c2ccc(-c3cccc4ccccc34)cc2)c2cccc(-n3c4ccccc4c4c5ccccc5ccc43)c2)cc1 ; c1ccc(-c2ccc(-c3cccc(N(c4ccccc4-c4ccc5ccccc5c4)c4ccc(-c5cccc6ccccc56)c5ccccc45)c3)cc2-n2c3ccccc3c3c4ccccc4ccc32)cc1]

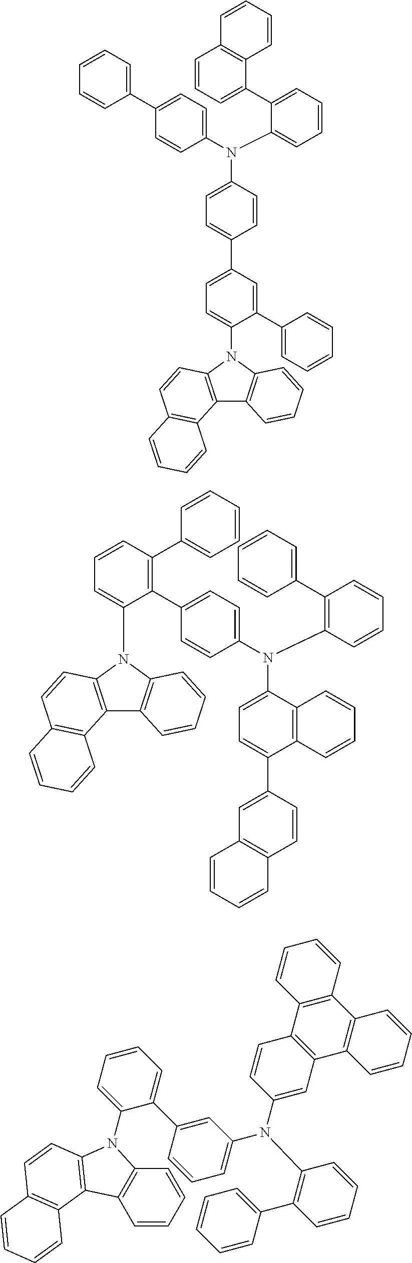

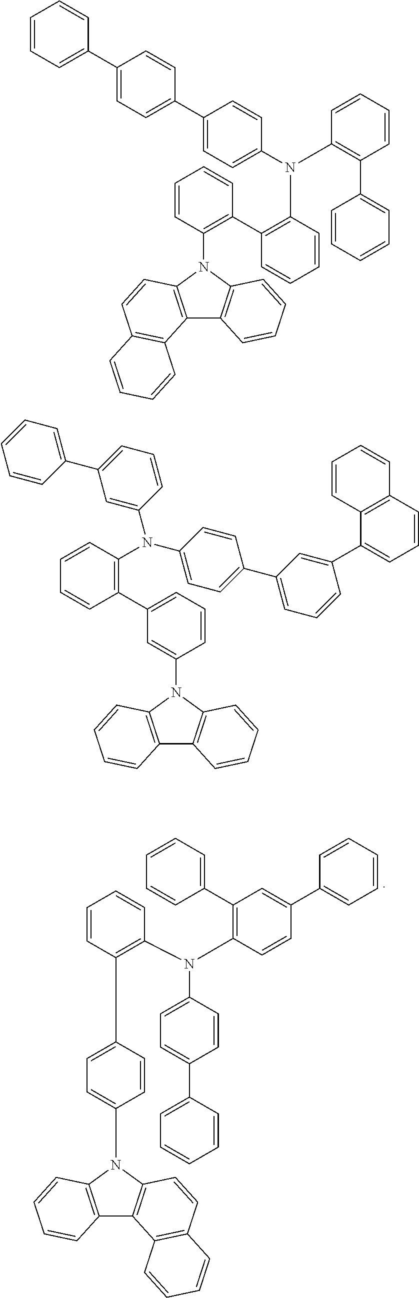 [SMILES: c1ccc(-c2ccc(-c3ccc(N(c4ccccc4-c4ccccc4)c4ccccc4-c4ccccc4-n4c5ccccc5c5c6ccccc6ccc54)cc3)cc2)cc1 ; c1ccc(-c2cccc(N(c3ccc(-c4cccc(-c5cccc6ccccc56)c4)cc3)c3ccccc3-c3cccc(-n4c5ccccc5c5ccccc54)c3)c2)cc1 ; c1ccc(-c2ccc(N(c3ccccc3-c3ccc(-n4c5ccccc5c5c6ccccc6ccc54)cc3)c3ccc(-c4ccccc4)cc3-c3ccccc3)cc2)cc1]

Among the above listed compounds, the compound represented by ‘[structural formula] Dₙ’ is a compound of which the corresponding ‘structural formula’ is substituted with n deuteriums.

The compound represented by Chemical Formula 2 can be prepared by a preparation method as shown in the following Reaction Scheme 2 as an example, and other remaining compounds can be prepared in a similar manner.

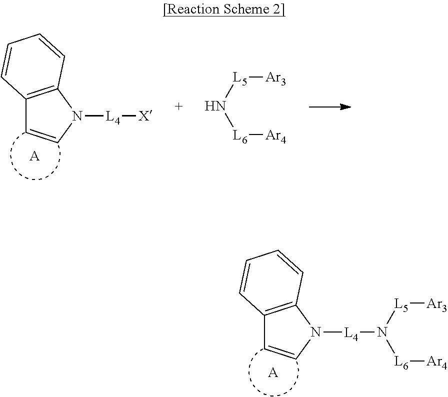

In Reaction Scheme 2, Ar₃, Ar₄ and L₄ to L₆ are the same as defined in Chemical Formula 2, and X′ is halogen, and preferably X′ is chloro or bromo.

Reaction Scheme 2 is an amine substitution reaction, which is preferably carried out in the presence of a palladium catalyst and a base, and a reactive group for the amine substitution reaction can be modified as known in the art. The preparation method can be further embodied in Preparation Examples described hereinafter.

Preferably, the weight ratio of the compound represented by Chemical Formula 1 and the compound represented by Chemical Formula 2 in the light emitting layer is 10:90 to 90:10, more preferably 20:80 to 80:20, 30:70 to 70:30, or 40:60 to 60:40.

Meanwhile, the light emitting layer may further include a dopant in addition to the host. The dopant material is not particularly limited as long as it is a material used for the organic light emitting device. As an example, an aromatic amine derivative, a styrylamine compound, a boron complex, a fluoranthene compound, a metal complex, and the like can be mentioned. Specific examples of the aromatic amine derivatives include substituted or unsubstituted fused aromatic ring derivatives having an arylamino group, examples thereof include pyrene, anthracene, chrysene, and periflanthene having the arylamino group, and the like. The styrylamine compound is a compound where at least one arylvinyl group is substituted in substituted or unsubstituted arylamine, in which one or two or more substituent groups selected from the group consisting of an aryl group, a silyl group, an alkyl group, a cycloalkyl group, and an arylamino group are substituted or unsubstituted. Specific examples thereof include styrylamine, styryldiamine, styryltriamine, styryltetramine, and the like, but are not limited thereto. Further, examples of the metal complex include an iridium complex, a platinum complex, and the like, but are not limited thereto.

Preferably, the dopant material may be one selected from the following, but is not limited thereto:

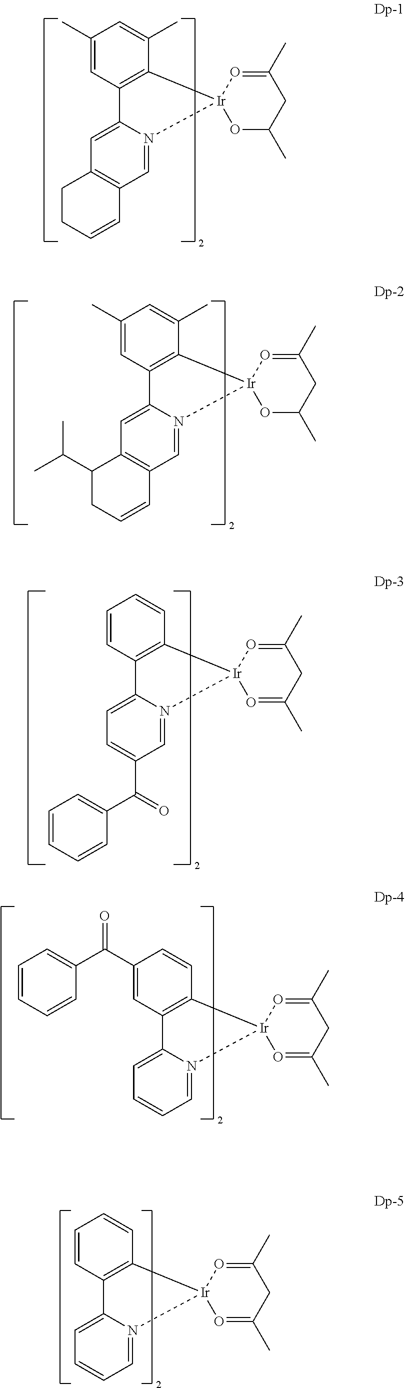

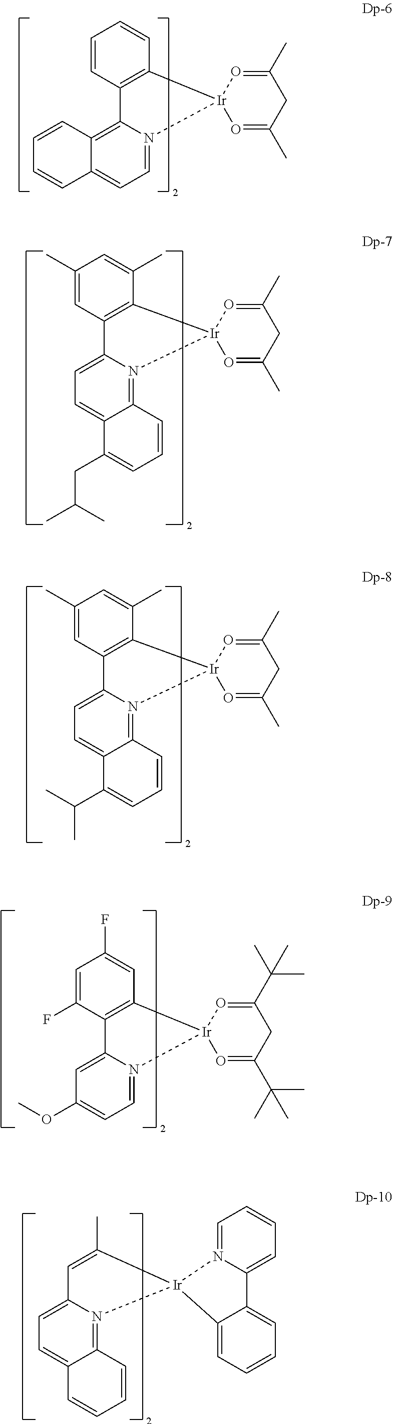

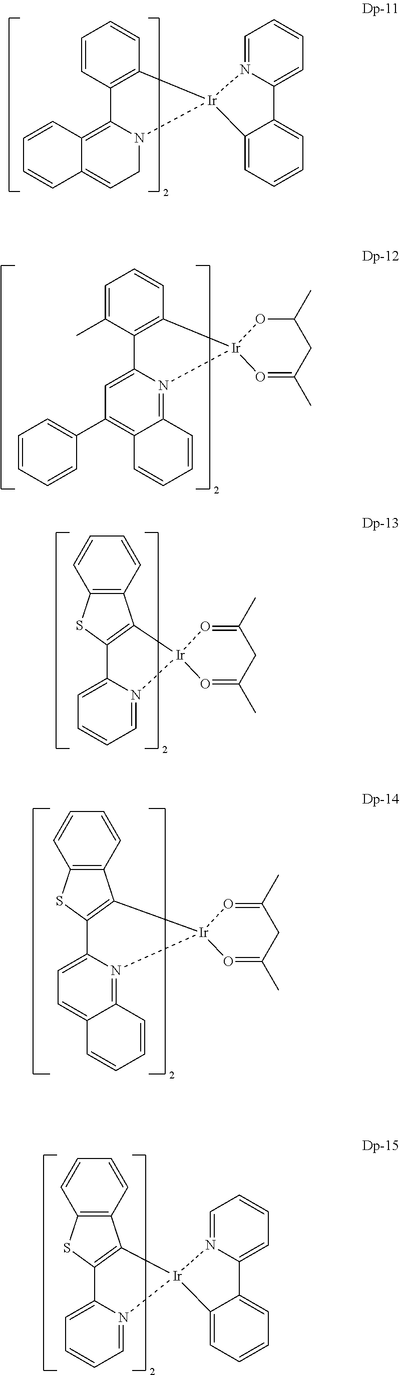

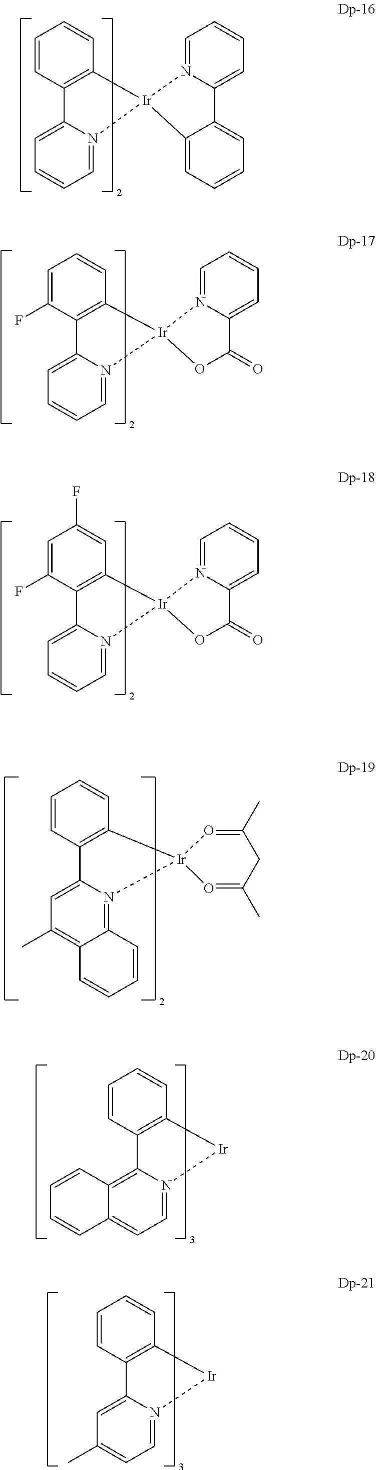

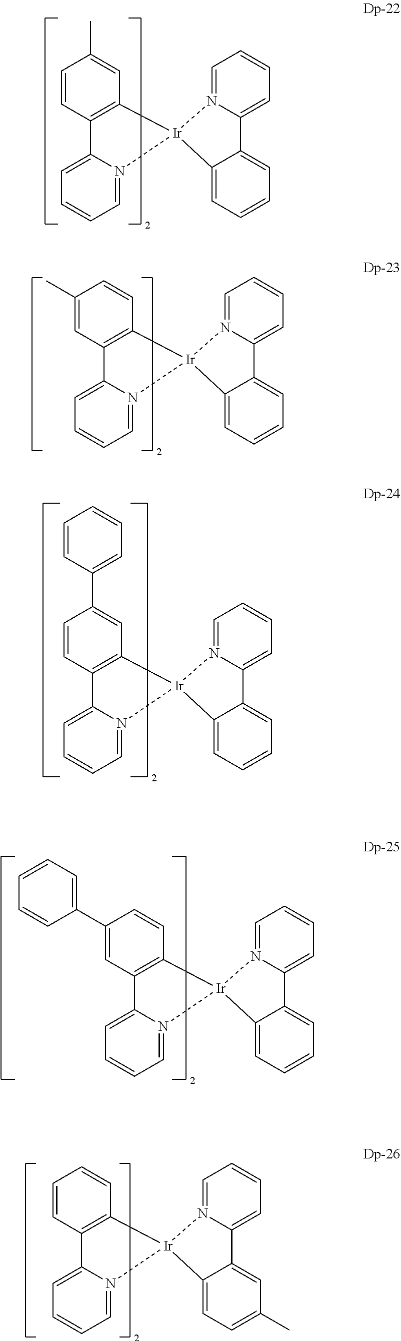

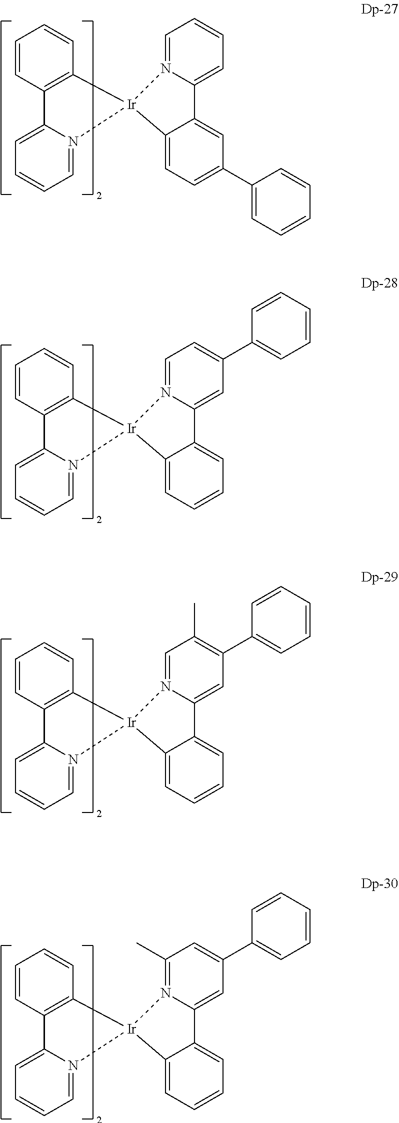

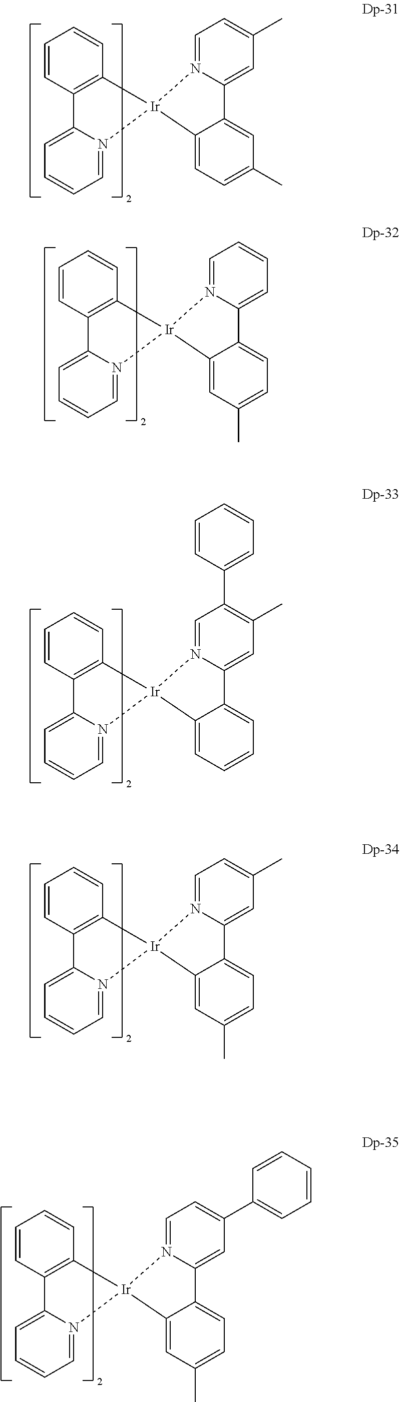

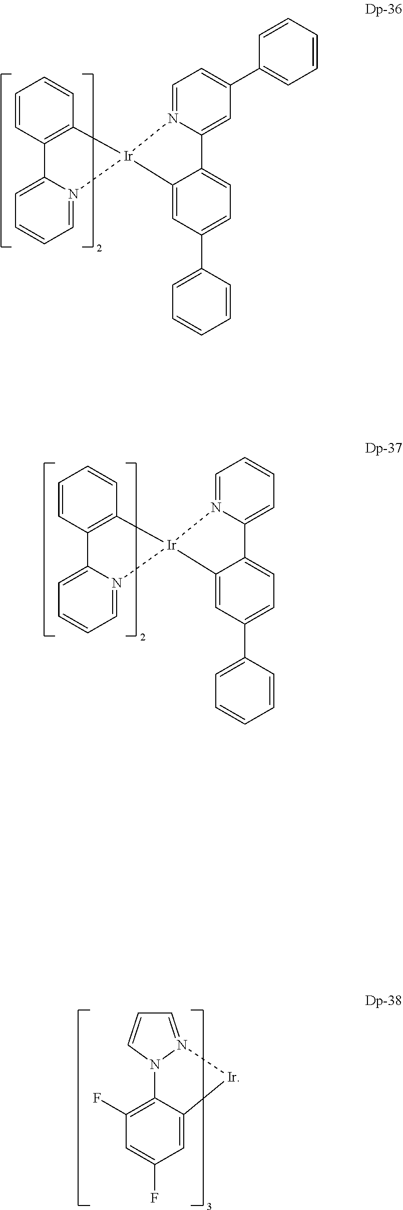

Hole Blocking Layer

The organic light emitting device according to the present disclosure may include a hole blocking layer between the light emitting layer and the electron transport layer described later, if necessary. The hole blocking layer refers to a layer which is formed on the light emitting layer, and preferably, is provided in contact with the light emitting layer, and thus severs to control electron mobility, to prevent excessive movement of holes, and to increase the probability of hole-electron bonding, thereby improving the efficiency of the organic light emitting device. The hole blocking layer includes a hole blocking material, and as an example of such hole blocking material, a compound into which an electron-withdrawing group is introduced, such as azine derivatives including triazine; triazole derivatives; oxadiazole derivatives; phenanthroline derivatives; phosphine oxide derivatives can be used, but is not limited thereto.

Electron Transport Layer

The organic light emitting device according to the present disclosure may include an electron transport layer on the light emitting layer, if necessary.

The electron transport layer is a layer that receives the electrons from the cathode or the electron injection layer formed on the cathode and transports the electrons to the light emitting layer, and that suppress the transfer of holes from the light emitting layer, and an electron transport material is suitably a material which may receive electrons well from a cathode and transfer the electrons to a light emitting layer, and has a large mobility for electrons.

Specific examples of the electron transport material include: an Al complex of 8-hydroxyquinoline; a complex including Alq₃; an organic radical compound; a hydroxyflavone-metal complex, and the like, but are not limited thereto. The electron transport layer may be used with any desired cathode material, as used according to a conventional technique. In particular, appropriate examples of the cathode material are a typical material which has a low work function, followed by an aluminum layer or a silver layer. Specific examples thereof include cesium, barium, calcium, ytterbium, and samarium, in each case followed by an aluminum layer or a silver layer.

Electron Injection Layer

The organic light emitting device according to the present disclosure may further include an electron injection layer on the light emitting layer (or on the electron transport layer, if the electron transport layer exists).

The electron injection layer is a layer which injects electrons from an electrode, and is preferably a compound which has a capability of transporting electrons, has an effect of injecting electrons from a cathode and an excellent effect of injecting electrons into a light emitting layer or a light emitting material, prevents excitons produced from the light emitting layer from moving to a hole injection layer, and is also excellent in the ability to form a thin film.

Specific examples of the electron injection layer include fluorenone, anthraquinodimethane, diphenoquinone, thiopyran dioxide, oxazole, oxadiazole, triazole, imidazole, perylenetetracarboxylic acid, fluorenylidene methane, anthrone, and the like, and derivatives thereof, a metal complex compound, a nitrogen-containing 5-membered ring derivative, and the like, but are not limited thereto.

Examples of the metal complex compound include 8-hydroxyquinolinato lithium, bis(8-hydroxyquinolinato)zinc, bis(8-hydroxyquinolinato)copper, bis(8-hydroxyquinolinato)manganese, tris(8-hydroxyquinolinato)aluminum, tris(2-methyl-8-hydroxyquinolinato)aluminum, tris(8-hydroxyquinolinato)gallium, bis(10-hydroxybenzo[h]quinolinato)beryllium, bis(10-hydroxybenzo[h]quinolinato)zinc, bis(2-methyl-8-quinolinato)chlorogallium, bis(2-methyl-8-quinolinato)(o-cresolato)gallium, bis(2-methyl-8-quinolinato)(1-naphtholato)aluminum, bis(2-methyl-8-quinolinato)(2-naphtholato)gallium, and the like, but are not limited thereto.

Meanwhile, in the present disclosure, the “electron injection and transport layer” is a layer that performs both the roles of the electron injection layer and the electron transport layer, and the materials that perform the roles of each layer may be used alone or in combination, without being limited thereto.

Organic Light Emitting Device

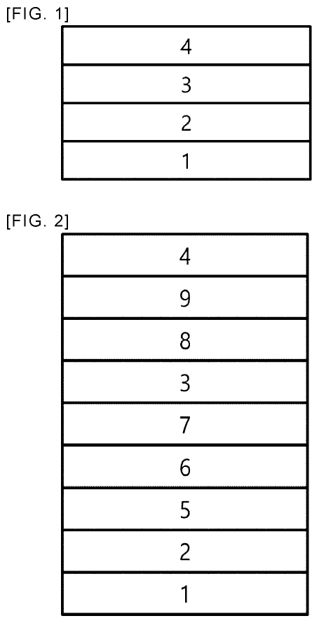

The structure of the organic light emitting device according to the present disclosure is illustrated in FIGS. 1 and 2. FIG. 1 shows an example of an organic light emitting device comprising a substrate 1, an anode 2, a light emitting layer 3, and a cathode 4. FIG. 2 shows an example of an organic light emitting device comprising a substrate 1, an anode 2, a hole injection layer 5, a hole transport layer 6, an electron blocking layer 7, a light emitting layer 3, a hole blocking layer 8, an electron injection and transport layer 9, and a cathode 4.

The organic light emitting device according to the present disclosure can be manufactured by sequentially stacking the above-described structures. In this case, the organic light emitting device may be manufactured by depositing a metal, metal oxides having conductivity, or an alloy thereof on the substrate by using a PVD (physical vapor deposition) method such as a sputtering method or an e-beam evaporation method to form the anode, forming the respective layers described above thereon, and then depositing a material that can be used as the cathode thereon. In addition to such a method, the organic light emitting device may be manufactured by sequentially depositing from the cathode material to the anode material on a substrate in the reverse order of the above-mentioned configuration (WO 2003/012890). Further, the light emitting layer may be formed by subjecting hosts and dopants to a vacuum deposition method and a solution coating method. Herein, the solution coating method means a spin coating, a dip coating, a doctor blading, an inkjet printing, a screen printing, a spray method, a roll coating, or the like, but is not limited thereto.

Meanwhile, the organic light emitting device according to the present disclosure may be a bottom emission device, a top emission device, or a double-sided light emitting device, and particularly, may be a bottom emission device that requires relatively high luminous efficiency.

Below, preferable embodiments are presented to assist in the understanding of the present disclosure. the following examples are only provided for a better understanding of the present disclosure, and is not intended to limit the content of the present disclosure.

### Synthesis Example 1-1

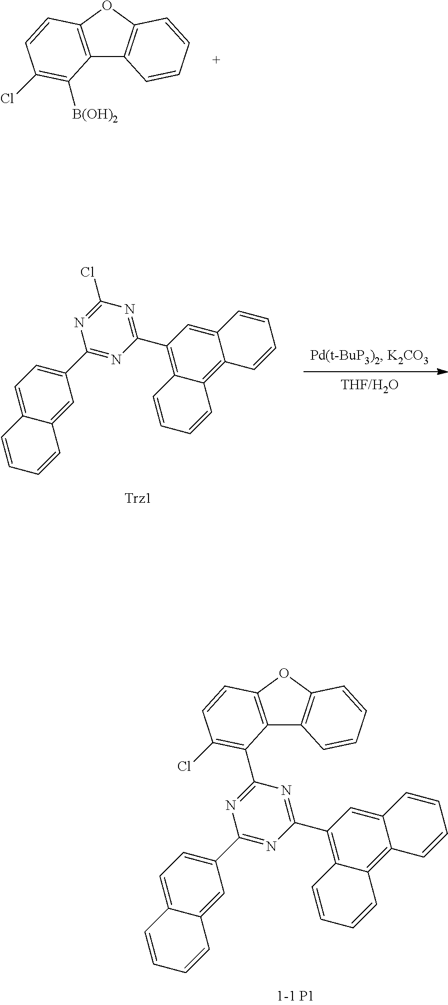 [SMILES: OB(O)c1c(Cl)ccc2oc3ccccc3c12 ; Clc1nc(-c2ccc3ccccc3c2)nc(-c2cc3ccccc3c3ccccc23)n1 ; "THF/H2O" ; Clc1ccc2oc3ccccc3c2c1-c1nc(-c2ccc3ccccc3c2)nc(-c2cc3ccccc3c3ccccc23)n1]

(2-Chlorodibenzo[b,d]furan-1-yl)boronic acid (15 g, 60.9 mmol) and Compound Trz1 (26.7 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred and then bis(tri-tert-butylphosphine)palladium(0) (0.3 g, 0.6 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 24.5 g of Compound 1-1_P1. (Yield: 69%, MS: [M+H]+=584).

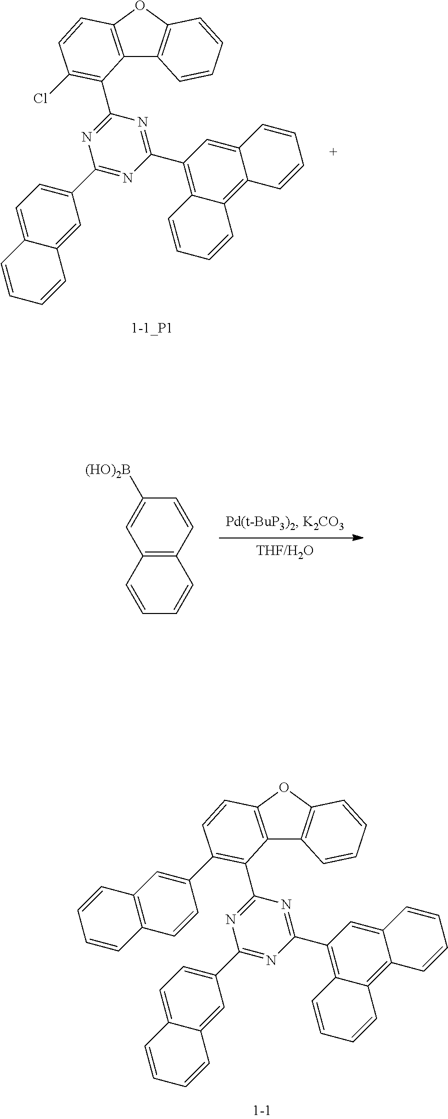 [SMILES: Clc1ccc2oc3ccccc3c2c1-c1nc(-c2ccc3ccccc3c2)nc(-c2cc3ccccc3c3ccccc23)n1 ; OB(O)c1ccc2ccccc2c1 ; "THF/H2O" ; c1ccc2cc(-c3nc(-c4cc5ccccc5c5ccccc45)nc(-c4c(-c5ccc6ccccc6c5)ccc5oc6ccccc6c45)n3)ccc2c1]

Compound 1-1_P1 (15 g, 25.7 mmol) and naphthalen-2-ylboronic acid (4.6 g, 27 mmol) were added to 300 ml of THF, and the mixture was stirred and refluxed. Then, potassium carbonate (10.6 g, 77 mmol) was dissolved in 32 ml of water and added thereto, and the mixture was sufficiently stirred and then bis(tri-tert-butylphosphine)palladium(0) (0.1 g, 0.3 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 12.1 g of Compound 1-1. (Yield: 70%, MS: [M+H]⁺=676).

### Synthesis Example 1-2

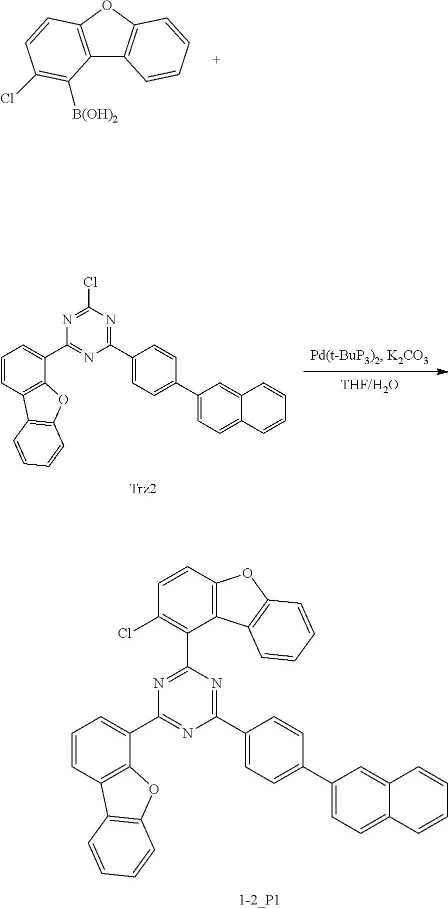 [SMILES: OB(O)c1c(Cl)ccc2oc3ccccc3c12 ; Clc1nc(-c2ccc(-c3ccc4ccccc4c3)cc2)nc(-c2cccc3c2oc2ccccc23)n1 ; "THF/H2O" ; Clc1ccc2oc3ccccc3c2c1-c1nc(-c2ccc(-c3ccc4ccccc4c3)cc2)nc(-c2cccc3c2oc2ccccc23)n1]

(2-Chlorodibenzo[b,d]furan-1-yl)boronic acid (15 g, 60.9 mmol) and Compound Trz2 (30.9 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred and then bis(tri-tert-butylphosphine)palladium(0) (0.3 g, 0.6 mmol) was added. After the reaction for 2 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 26.5 g of Compound 1-2_P1. (Yield: 67%, MS: [M+H]⁺=650).

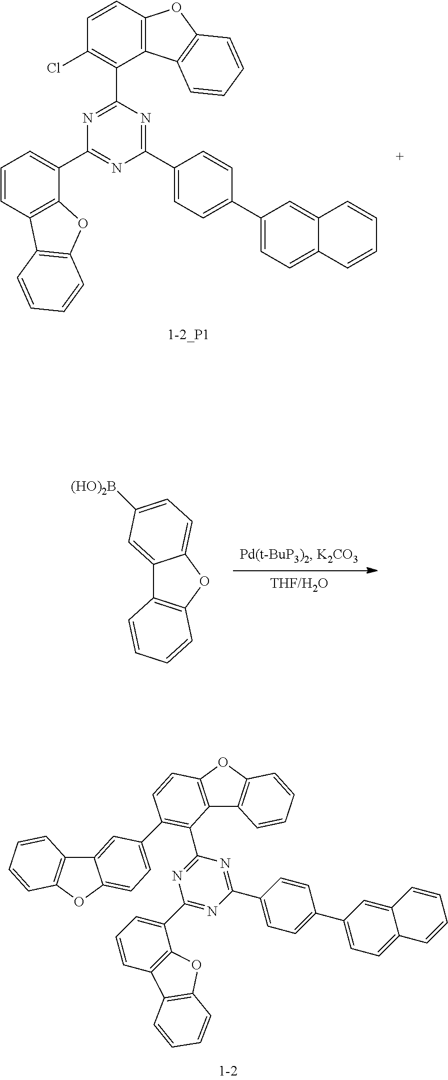 [SMILES: Clc1ccc2oc3ccccc3c2c1-c1nc(-c2ccc(-c3ccc4ccccc4c3)cc2)nc(-c2cccc3c2oc2ccccc23)n1 ; OB(O)c1ccc2oc3ccccc3c2c1 ; "THF/H2O" ; c1ccc2cc(-c3ccc(-c4nc(-c5cccc6c5oc5ccccc56)nc(-c5c(-c6ccc7oc8ccccc8c7c6)ccc6oc7ccccc7c56)n4)cc3)ccc2c1]

Compound 1-2_P1 (15 g, 23.1 mmol) and dibenzo[b,d]furan-2-ylboronic acid (5.1 g, 24.2 mmol) were added to 300 ml of THF, and the mixture was stirred and refluxed. Then, potassium carbonate (9.6 g, 69.2 mmol) was dissolved in 29 ml of water and added thereto, and the mixture was sufficiently stirred and then bis(tri-tert-butylphosphine)palladium(0) (0.1 g, 0.2 mmol) was added. After the reaction for 2 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 13.2 g of Compound 1-2. (Yield: 73%, MS: [M+H]⁺=782).

### Synthesis Example 1-3

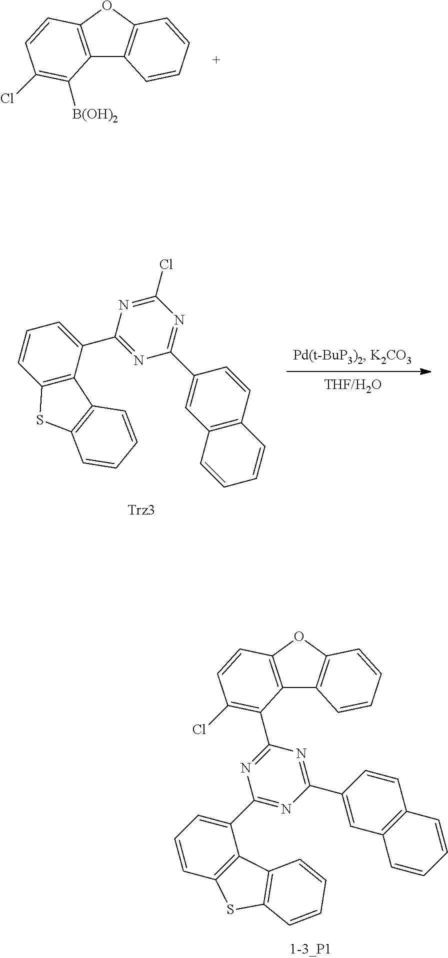 [SMILES: OB(O)c1c(Cl)ccc2oc3ccccc3c12 ; Clc1nc(-c2ccc3ccccc3c2)nc(-c2cccc3sc4ccccc4c23)n1 ; "THF/H2O" ; Clc1ccc2oc3ccccc3c2c1-c1nc(-c2ccc3ccccc3c2)nc(-c2cccc3sc4ccccc4c23)n1]

(2-Chlorodibenzo[b,d]furan-1-yl)boronic acid (15 g, 60.9 mmol) and Compound Trz3 (27.1 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred and then bis(tri-tert-butylphosphine)palladium(0) (0.3 g, 0.6 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 23.3 g of Compound 1-3_P1. (Yield: 66%, MS: [M+H]⁺=580).

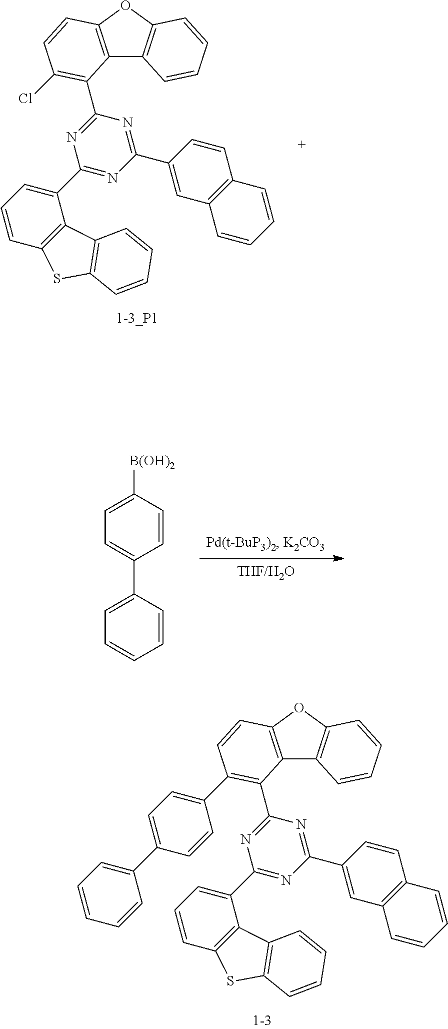 [SMILES: Clc1ccc2oc3ccccc3c2c1-c1nc(-c2ccc3ccccc3c2)nc(-c2cccc3sc4ccccc4c23)n1 ; OB(O)c1ccc(-c2ccccc2)cc1 ; "THF/H2O" ; c1ccc(-c2ccc(-c3ccc4oc5ccccc5c4c3-c3nc(-c4ccc5ccccc5c4)nc(-c4cccc5sc6ccccc6c45)n3)cc2)cc1]

Compound 1-3_P1 (15 g, 25.4 mmol) and [1,1′-biphenyl]-4-ylboronic acid (5.3 g, 26.7 mmol) were added to 300 ml of THF, and the mixture was stirred and refluxed. Then, potassium carbonate (10.5 g, 76.3 mmol) was dissolved in 32 ml of water and added thereto, and the mixture was sufficiently stirred and then bis(tri-tert-butylphosphine)palladium(0) (0.1 g, 0.3 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 13.5 g of Compound 1-3. (Yield: 75%, MS: [M+H]⁺=708).

### Synthesis Example 1-4

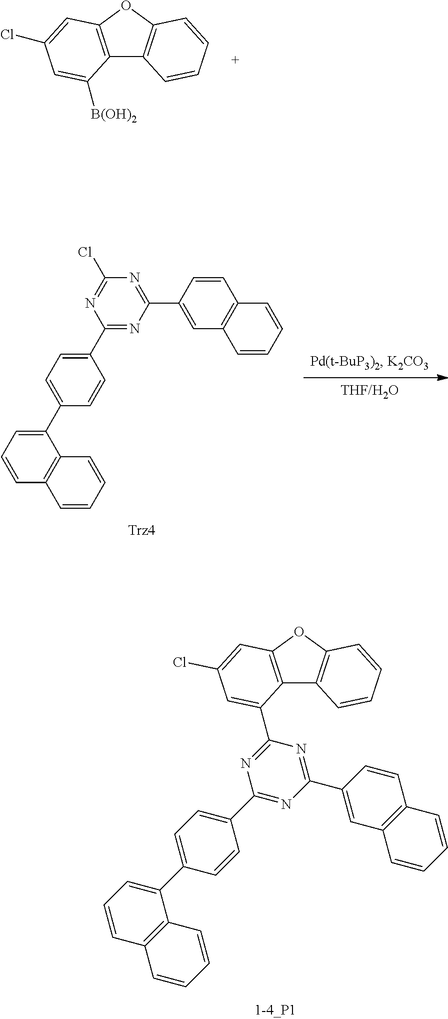 [SMILES: OB(O)c1cc(Cl)cc2oc3ccccc3c12 ; Clc1nc(-c2ccc(-c3cccc4ccccc34)cc2)nc(-c2ccc3ccccc3c2)n1 ; "THF/H2O" ; Clc1cc(-c2nc(-c3ccc(-c4cccc5ccccc45)cc3)nc(-c3ccc4ccccc4c3)n2)c2c(c1)oc1ccccc12]

(3-chlorodibenzo[b,d]furan-1-yl)boronic acid (15 g, 60.9 mmol) and Compound Trz4 (28.4 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred and then bis(tri-tert-butylphosphine)palladium(0) (0.3 g, 0.6 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 27.4 g of Compound 1-4_P1. (Yield: 74%, MS: [M+H]⁺=610).

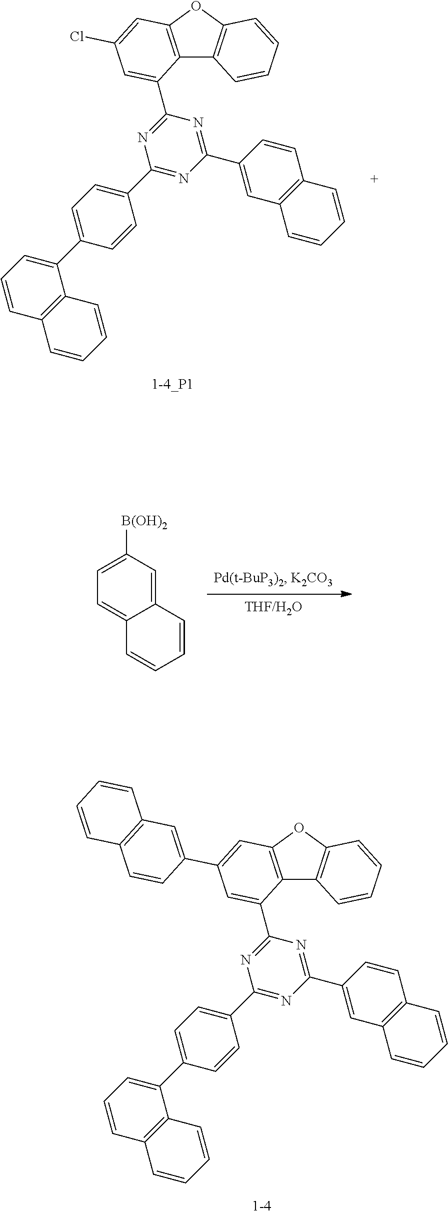 [SMILES: Clc1cc(-c2nc(-c3ccc(-c4cccc5ccccc45)cc3)nc(-c3ccc4ccccc4c3)n2)c2c(c1)oc1ccccc12 ; OB(O)c1ccc2ccccc2c1 ; "THF/H2O" ; c1ccc2cc(-c3cc(-c4nc(-c5ccc(-c6cccc7ccccc67)cc5)nc(-c5ccc6ccccc6c5)n4)c4c(c3)oc3ccccc34)ccc2c1]

Compound 1-4_P1 (15 g, 24.6 mmol) and naphthalen-2-ylboronic acid (4.4 g, 25.8 mmol) were added to 300 ml of THF, and the mixture was stirred and refluxed. Then, potassium carbonate (10.2 g, 73.8 mmol) was dissolved in 31 ml of water and added thereto, and the mixture was sufficiently stirred and then bis(tri-tert-butylphosphine)palladium(0) (0.1 g, 0.2 mmol) was added After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 11.2 g of Compound 1-4. (Yield: 65%, MS: [M+H]⁺=702).

### Synthesis Example 1-5

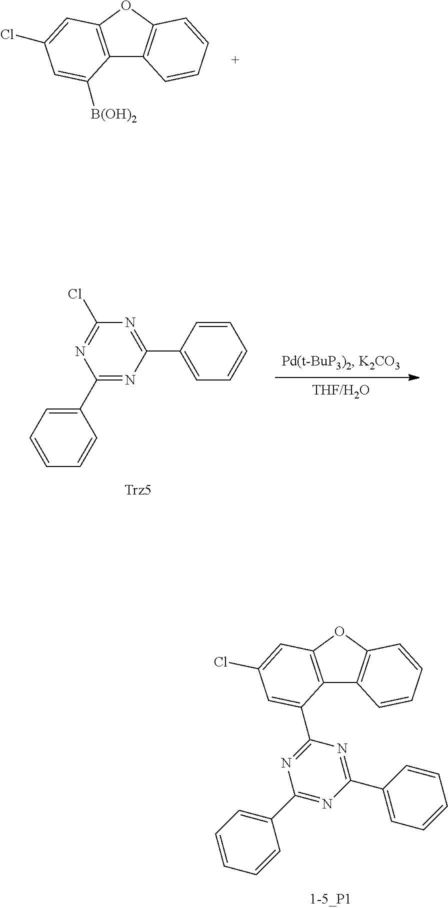 [SMILES: OB(O)c1cc(Cl)cc2oc3ccccc3c12 ; Clc1nc(-c2ccccc2)nc(-c2ccccc2)n1 ; "THF/H2O" ; Clc1cc(-c2nc(-c3ccccc3)nc(-c3ccccc3)n2)c2c(c1)oc1ccccc12]

(3-Chlorodibenzo[b,d] furan-1-yl)boronic acid (15 g, 60.9 mmol) and Compound Trz5 (17.1 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred and then bis(tri-tert-butylphosphine)palladium(0) (0.3 g, 0.6 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 18.7 g of Compound 1-5_P1. (Yield: 71%, MS: [M+H]⁺=434).

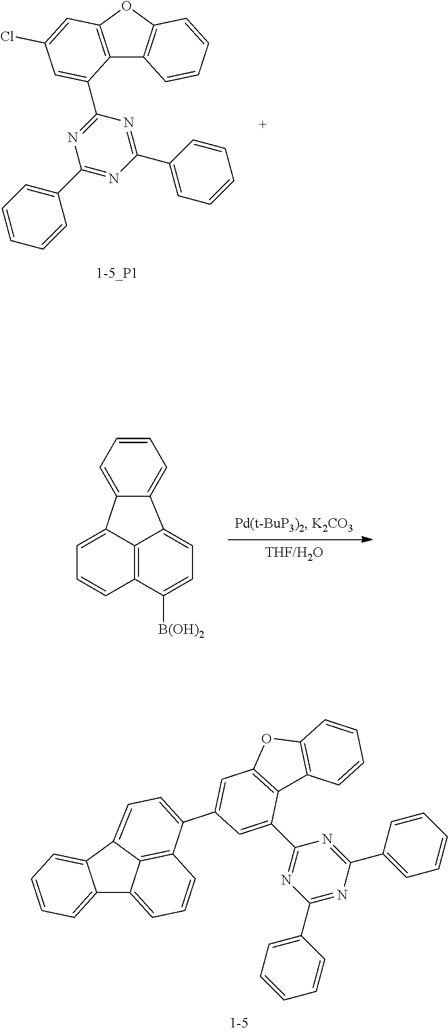 [SMILES: Clc1cc(-c2nc(-c3ccccc3)nc(-c3ccccc3)n2)c2c(c1)oc1ccccc12 ; OB(O)c1ccc2c3c(cccc13)-c1ccccc1-2 ; "THF/H2O" ; c1ccc(-c2nc(-c3ccccc3)nc(-c3cc(-c4ccc5c6c(cccc46)-c4ccccc4-5)cc4oc5ccccc5c34)n2)cc1]

Compound 1-5_P1 (15 g, 34.6 mmol) and fluoranthen-3-ylboronic acid (8.9 g, 36.3 mmol) were added to 300 ml of THF, and the mixture was stirred and refluxed. Then, potassium carbonate (14.3 g, 103.7 mmol) was dissolved in 43 ml of water and added thereto, and the mixture was sufficiently stirred and then bis(tri-tert-butylphosphine)palladium(0) (0.2 g, 0.3 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 14.9 g of Compound 1-5. (Yield: 72%, MS: [M+H]⁺=600).

### Synthesis Example 1-6

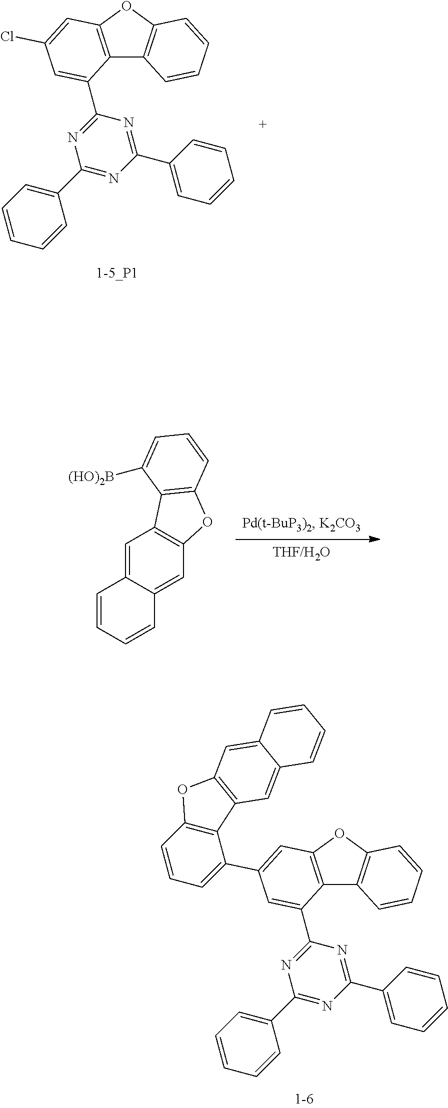 [SMILES: Clc1cc(-c2nc(-c3ccccc3)nc(-c3ccccc3)n2)c2c(c1)oc1ccccc12 ; OB(O)c1cccc2oc3cc4ccccc4cc3c12 ; "THF/H2O" ; c1ccc(-c2nc(-c3ccccc3)nc(-c3cc(-c4cccc5oc6cc7ccccc7cc6c45)cc4oc5ccccc5c34)n2)cc1]

Compound 1-5_P1 (15 g, 34.6 mmol) and naphtho[2,3-b]benzofuran-1-ylboronic acid (9.5 g, 36.3 mmol) were added to 300 ml of THF, and the mixture was stirred and refluxed. Then, potassium carbonate (14.3 g, 103.7 mmol) was dissolved in 43 ml of water and added thereto, and the mixture was sufficiently stirred and then bis(tri-tert-butylphosphine)palladium(0) (0.2 g, 0.3 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 13.8 g of Compound 1-6. (Yield: 65%, MS: [M+H]⁺=616).

### Synthesis Example 1-7

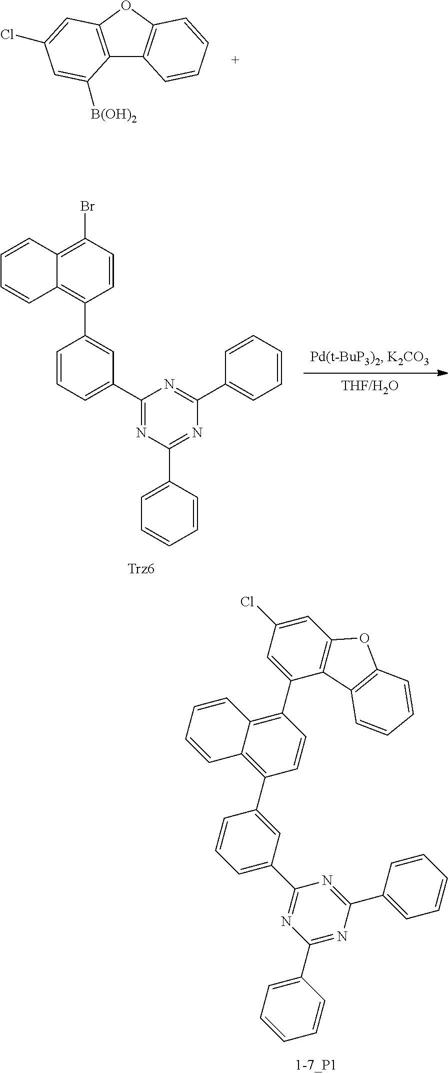 [SMILES: OB(O)c1cc(Cl)cc2oc3ccccc3c12 ; Brc1ccc(-c2cccc(-c3nc(-c4ccccc4)nc(-c4ccccc4)n3)c2)c2ccccc12 ; "THF/H2O" ; Clc1cc(-c2ccc(-c3cccc(-c4nc(-c5ccccc5)nc(-c5ccccc5)n4)c3)c3ccccc23)c2c(c1)oc1ccccc12]

(3-Chlorodibenzo[b,d]furan-1-yl)boronic acid (15 g, 60.9 mmol) and Compound Trz6 (32.9 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred and then bis(tri-tert-butylphosphine)palladium(0) (0.3 g, 0.6 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 27.4 g of Compound 1-7_P1. (Yield: 71%, MS: [M+H]⁺=636).

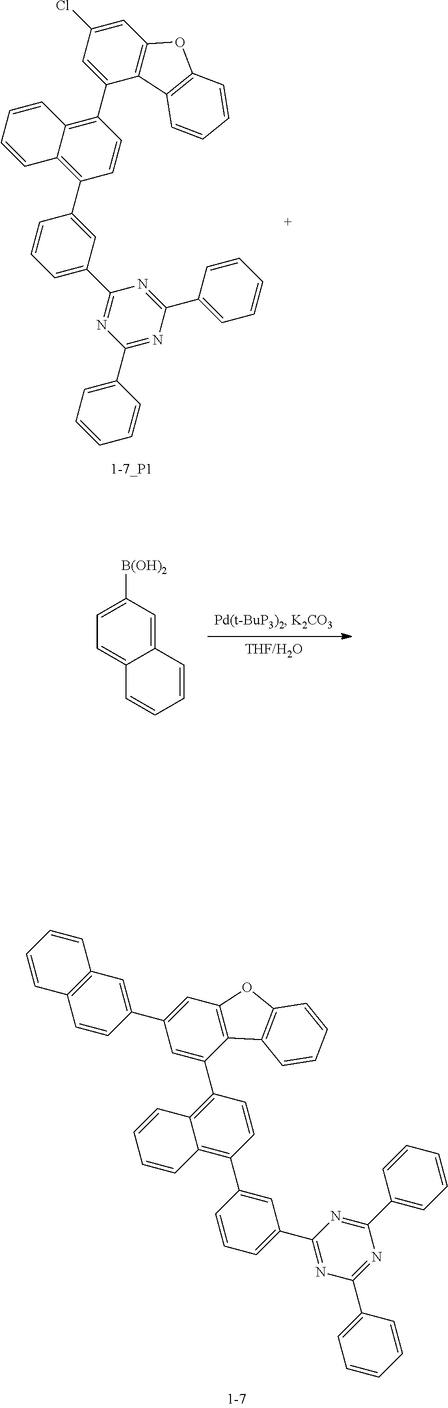 [SMILES: Clc1cc(-c2ccc(-c3cccc(-c4nc(-c5ccccc5)nc(-c5ccccc5)n4)c3)c3ccccc23)c2c(c1)oc1ccccc12 ; OB(O)c1ccc2ccccc2c1 ; "THF/H2O" ; c1ccc(-c2nc(-c3ccccc3)nc(-c3cccc(-c4ccc(-c5cc(-c6ccc7ccccc7c6)cc6oc7ccccc7c56)c5ccccc45)c3)n2)cc1]

Compound 1-7_P1 (15 g, 23.6 mmol) and naphthalen-2-ylboronic acid (4.3 g, 24.8 mmol) were added to 300 ml of THF, and the mixture was stirred and refluxed. Then, potassium carbonate (9.8 g, 70.7 mmol) was dissolved in 29 ml of water and added thereto, and the mixture was sufficiently stirred and then bis(tri-tert-butylphosphine)palladium(0) (0.1 g, 0.2 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 11.5 g of Compound 1-7. (Yield: 67%, MS: [M+H]⁺=728).

### Synthesis Example 1-8

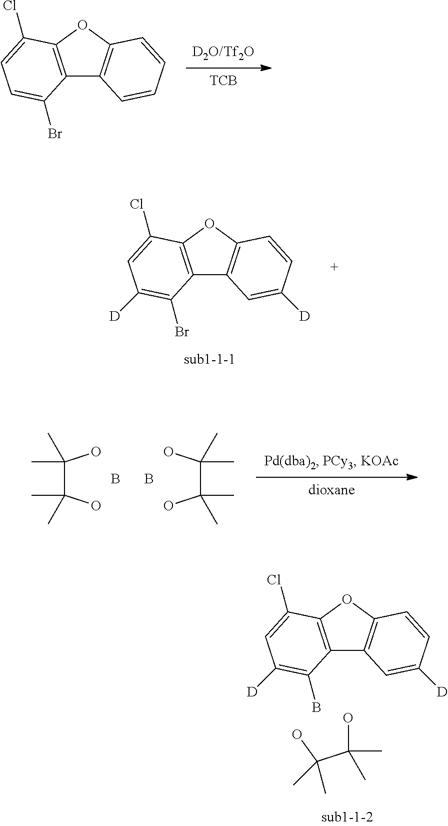

Trifluoromethanesulfonic anhydride (30.1 g, 106.6 mmol) and deuterium oxide (10.7 g, 532.8 mmol) were added at 0° C. and stirred for 5 hours to prepare a solution. 1-Bromo-4-chlorodibenzo[b,d]furan (15 g, 53.3 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of 1-bromo-4-chlorodibenzo[b,d]furan and 1,2,4-trichlorobenzene, and the mixture was stirred while heating up to 140° C. and then keeping that temperature. After the reaction for 3 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 6.5 g of compound sub1-1-1. (Yield: 43%, MS: [M+H]⁺=283)

Compound Sub1-1-1 (15 g, 52.9 mmol) and bis(pinacolato)diboron (14.8 g, 58.2 mmol) were added to 300 ml of 1,4-dioxane, and the mixture was stirred under reflux. Then, potassium acetate (7.8 g, 79.4 mmol) was added thereto, the mixture was sufficiently stirred, and then bis(dibenzylideneacetone)palladium(0) (0.9 g, 1.6 mmol) and tricyclohexylphosphine (0.9 g, 3.2 mmol) were added. After the reaction for 6 hours, the reaction mixture was cooled to room temperature, the organic layer was separated using chloroform and water, and then the organic layer was distilled. This was dissolved again in chloroform, washed twice with water, and the organic layer was then separated. Anhydrous magnesium sulfate was added thereto, stirred, then filtered, and the filtrate was distilled under reduced pressure. The concentrated compound was purified by silica gel column chromatography to prepare 10.8 g of Compound Sub1-1-2. (Yield: 62%, MS: [M+H]+=331)

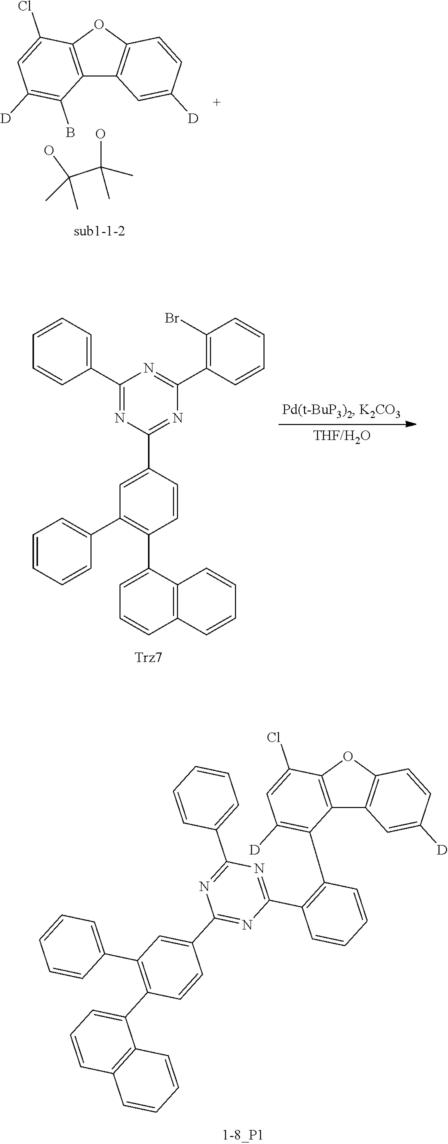

Compound Sub1-1-2 (15 g, 45.4 mmol) and Compound Trz7 (28.1 g, 47.6 mmol) were added to 300 ml of THF, and the mixture was stirred and refluxed. Then, potassium carbonate (18.8 g, 136.1 mmol) was dissolved in 56 ml of water and added thereto, and the mixture was sufficiently stirred and then bis(tri-tert-butylphosphine)palladium(0) (0.2 g, 0.5 mmol) was added. After the reaction for 2 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 23.3 g of Compound 1-8_P1. (Yield: 72%, MS: [M+H]⁺=714).

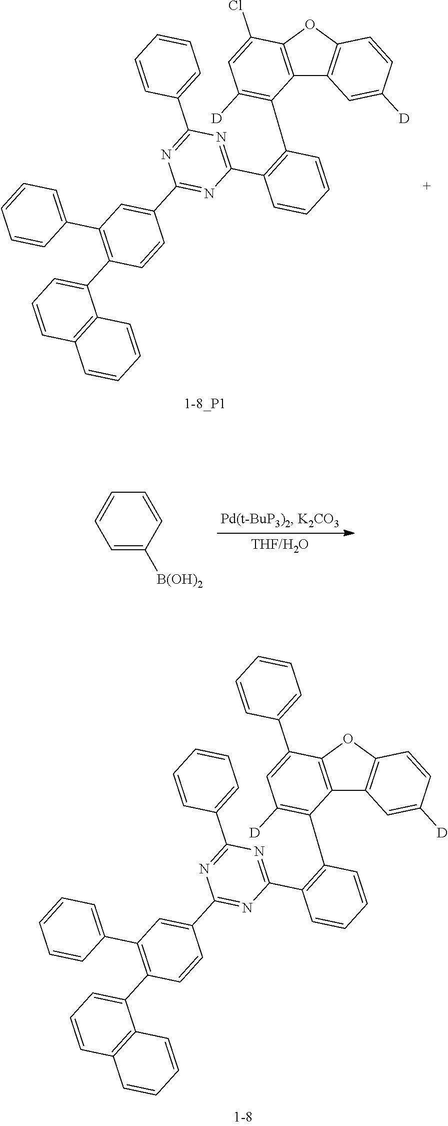 [SMILES: [2H]c1ccc2oc3c(Cl)cc([2H])c(-c4ccccc4-c4nc(-c5ccccc5)nc(-c5ccc(-c6cccc7ccccc67)c(-c6ccccc6)c5)n4)c3c2c1 ; OB(O)c1ccccc1 ; [2H]c1ccc2oc3c(-c4ccccc4)cc([2H])c(-c4ccccc4-c4nc(-c5ccccc5)nc(-c5ccc(-c6cccc7ccccc67)c(-c6ccccc6)c5)n4)c3c2c1]

Compound 1-8_P1 (15 g, 21 mmol) and phenylboronic acid (2.7 g, 22.1 mmol) were added to 300 ml of THF, and the mixture was stirred and refluxed. Then, potassium carbonate (8.7 g, 63 mmol) was dissolved in 26 ml of water and added thereto, and the mixture was sufficiently stirred and then bis(tri-tert-butylphosphine)palladium(0) (0.1 g, 0.2 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 10 g of Compound 1-8. (Yield: 63%, MS: [M+H]⁺=756).

### Synthesis Example 1-9

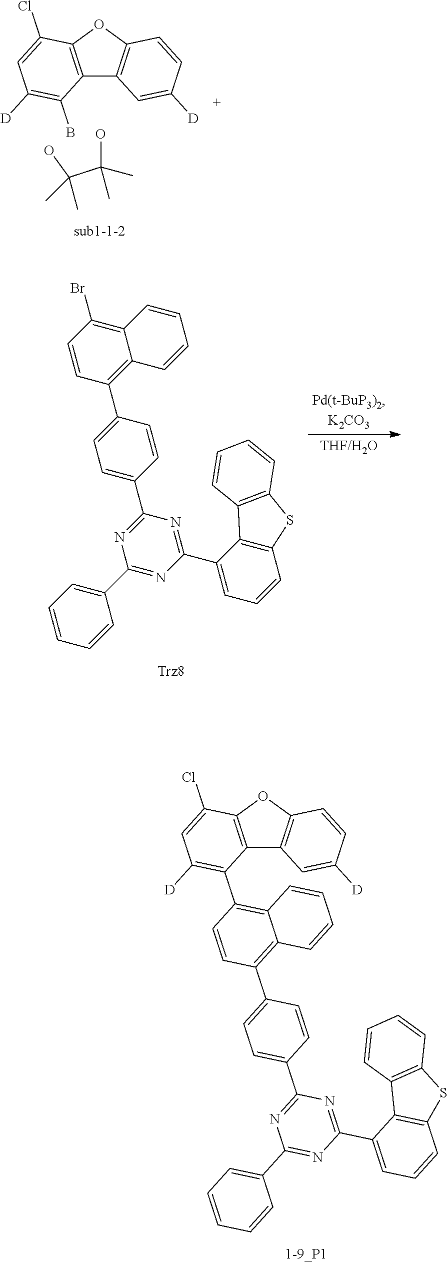

Compound Sub1-1-2 (15 g, 45.4 mmol) and Compound Trz8 (29.6 g, 47.6 mmol) were added to 300 ml of THF, and the mixture was stirred and refluxed. Then, potassium carbonate (18.8 g, 136.1 mmol) was dissolved in 56 ml of water and added thereto, and the mixture was sufficiently stirred and then bis(tri-tert-butylphosphine)palladium(0) (0.2 g, 0.5 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 25.3 g of Compound 1-9_P1. (Yield: 75%, MS: [M+H]⁺=744).

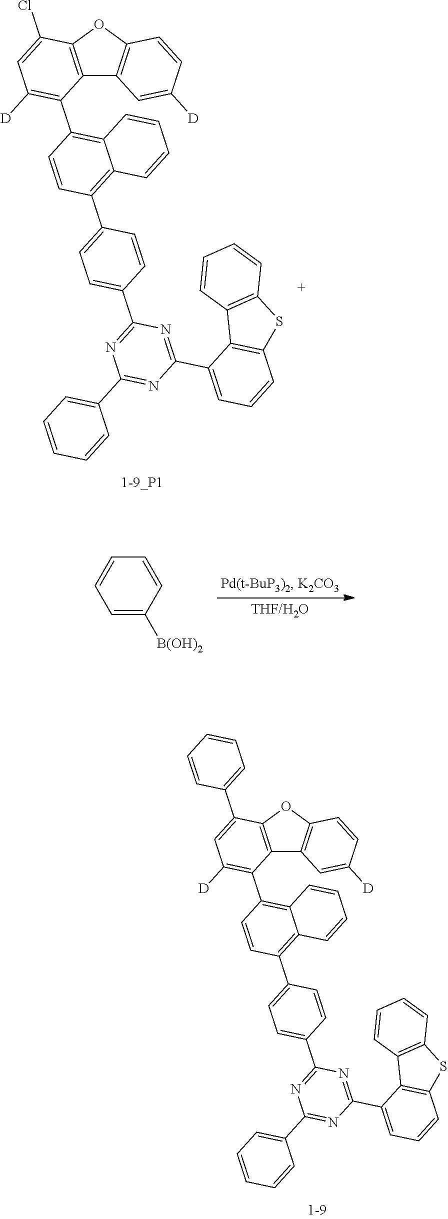 [SMILES: [2H]c1ccc2oc3c(Cl)cc([2H])c(-c4ccc(-c5ccc(-c6nc(-c7ccccc7)nc(-c7cccc8sc9ccccc9c78)n6)cc5)c5ccccc45)c3c2c1 ; OB(O)c1ccccc1 ; [2H]c1ccc2oc3c(-c4ccccc4)cc([2H])c(-c4ccc(-c5ccc(-c6nc(-c7ccccc7)nc(-c7cccc8sc9ccccc9c78)n6)cc5)c5ccccc45)c3c2c1]

Compound 1-9_P1 (15 g, 20.2 mmol) and phenylboronic acid (2.6 g, 21.2 mmol) were added to 300 ml of THF, and the mixture was stirred and refluxed. Then, potassium carbonate (8.4 g, 60.5 mmol) was dissolved in 25 ml of water and added thereto, and the mixture was sufficiently stirred and then bis(tri-tert-butylphosphine)palladium(0) (0.1 g, 0.2 mmol) was added After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 11.1 g of Compound 1-9. (Yield: 70%, MS: [M+H]⁺=786).

### Synthesis Example 1-10

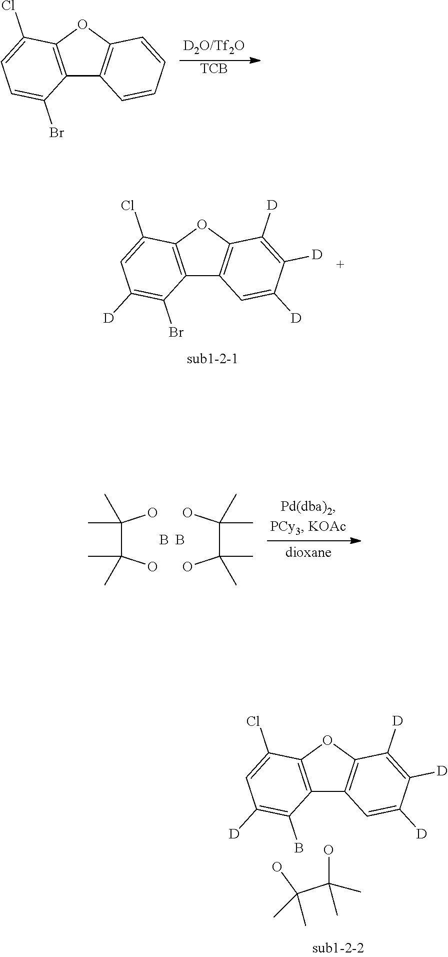

Trifluoromethanesulfonic anhydride (60.1 g, 213.1 mmol) and deuterium oxide (21.4 g, 1065.6 mmol) were added at 0° C. and stirred for hours to prepare a solution. 1-Bromo-4-chlorodibenzo[b,d]furan (15 g, 53.3 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of 1-bromo-4-chlorodibenzo[b,d]furan and 1,2,4-trichlorobenzene, and the mixture was stirred while heating up to 140° C. and then keeping that temperature. After the reaction for 10 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 5.3 g of compound Sub1-2-1. (Yield: 35%, MS: [M+H]⁺=285)

Compound Sub1-2-1 (15 g, 52.5 mmol) and bis(pinacolato)diboron (14.7 g, 57.8 mmol) were added to 300 ml of 1,4-dioxane, and the mixture was stirred under reflux. Then, potassium acetate (7.7 g, 78.8 mmol) was added thereto, the mixture was sufficiently stirred, and then bis(dibenzylideneacetone) palladium(0) (0.9 g, 1.6 mmol) and tricyclohexylphosphine (0.9 g, 3.2 mmol) were added. After the reaction for 6 hours, the reaction mixture was cooled to room temperature, the organic layer was separated using chloroform and water, and then the organic layer was distilled. This was dissolved again in chloroform, washed twice with water, and the organic layer was then separated. Anhydrous magnesium sulfate was added thereto, stirred, then filtered, and the filtrate was distilled under reduced pressure. The concentrated compound was purified by silica gel column chromatography to prepare 11 g of Compound Sub1-2-2. (Yield: 63%, MS: [M+H]⁺=333)

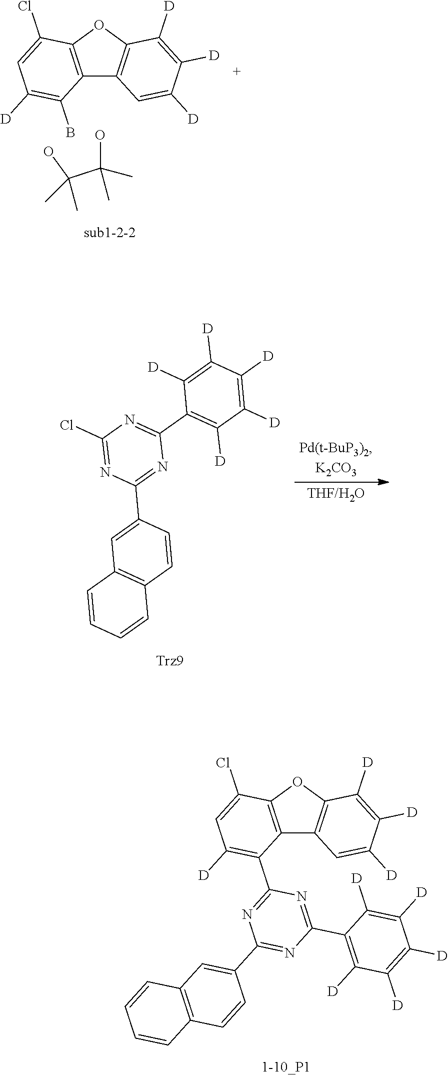

Compound Sub1-2-2 (15 g, 45.1 mmol) and Compound Trz9 (15.8 g, 47.4 mmol) were added to 300 ml of THF, and the mixture was stirred and refluxed. Then, potassium carbonate (18.7 g, 135.3 mmol) was dissolved in 56 ml of water and added thereto, and the mixture was sufficiently stirred and then bis(tri-tert-butylphosphine)palladium(0) (0.2 g, 0.5 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 14.6 g of Compound 1-10_P1. (Yield: 66%, MS: [M+H]⁺=493).

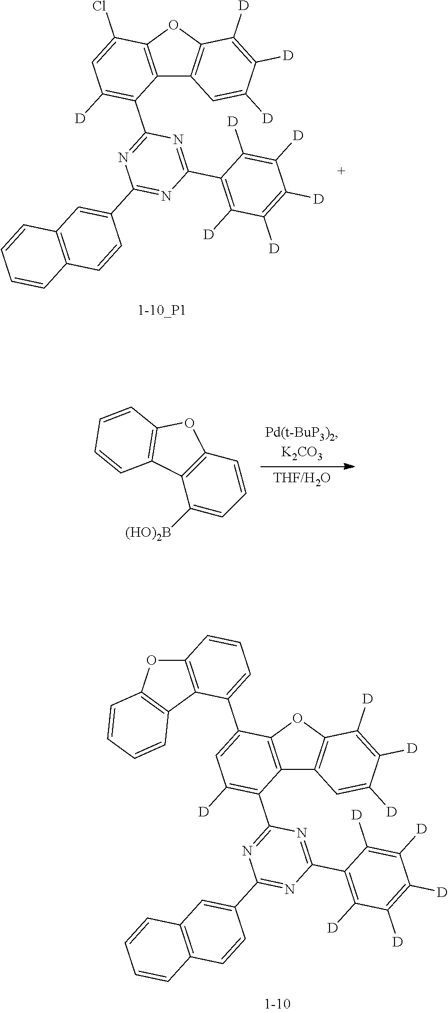 [SMILES: [2H]c1cc2c(oc3c(Cl)cc([2H])c(-c4nc(-c5ccc6ccccc6c5)nc(-c5c([2H])c([2H])c([2H])c([2H])c5[2H])n4)c32)c([2H])c1[2H] ; OB(O)c1cccc2oc3ccccc3c12 ; [2H]c1cc2c(oc3c(-c4cccc5oc6ccccc6c45)cc([2H])c(-c4nc(-c5ccc6ccccc6c5)nc(-c5c([2H])c([2H])c([2H])c([2H])c5[2H])n4)c32)c([2H])c1[2H]]

Compound 1-10_P1 (15 g, 30.4 mmol) and dibenzo[b,d]furan-1-ylboronic acid (6.8 g, 31.9 mmol) were added to 300 ml of THF, and the mixture was stirred and refluxed. Then, potassium carbonate (12.6 g, 91.3 mmol) was dissolved in 38 ml of water and added thereto, and the mixture was sufficiently stirred and then bis(tri-tert-butylphosphine)palladium(0) (0.2 g, 0.3 mmol) was added. After the reaction for 2 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 13.3 g of Compound 1-10. (Yield: 70%, MS: [M+H]⁺=625).

### Synthesis Example 1-11

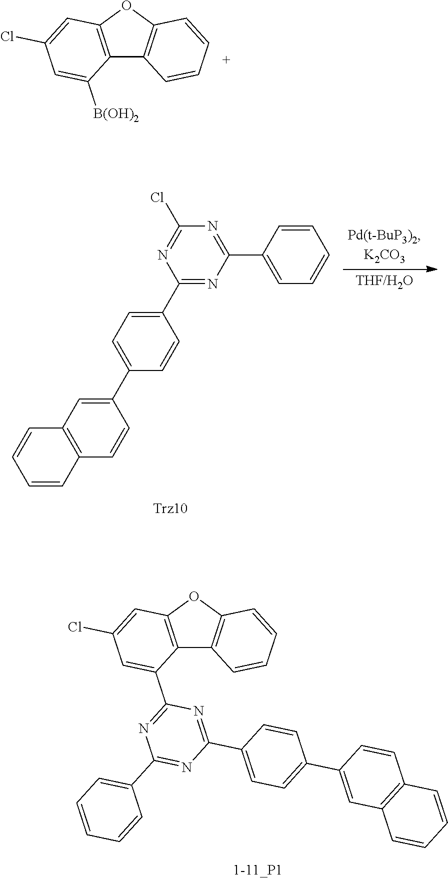 [SMILES: OB(O)c1cc(Cl)cc2oc3ccccc3c12 ; Clc1nc(-c2ccccc2)nc(-c2ccc(-c3ccc4ccccc4c3)cc2)n1 ; Clc1cc(-c2nc(-c3ccccc3)nc(-c3ccc(-c4ccc5ccccc5c4)cc3)n2)c2c(c1)oc1ccccc12]

(3-Chlorodibenzo[b,d]furan-1-yl)boronic acid (15 g, 60.9 mmol) and Compound Trz10 (25.2 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred and then bis(tri-tert-butylphosphine)palladium(0) (0.3 g, 0.6 mmol) was added. After the reaction for 2 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 21.1 g of Compound 1-11_P1. (Yield: 62%, MS: [M+H]⁺=560).

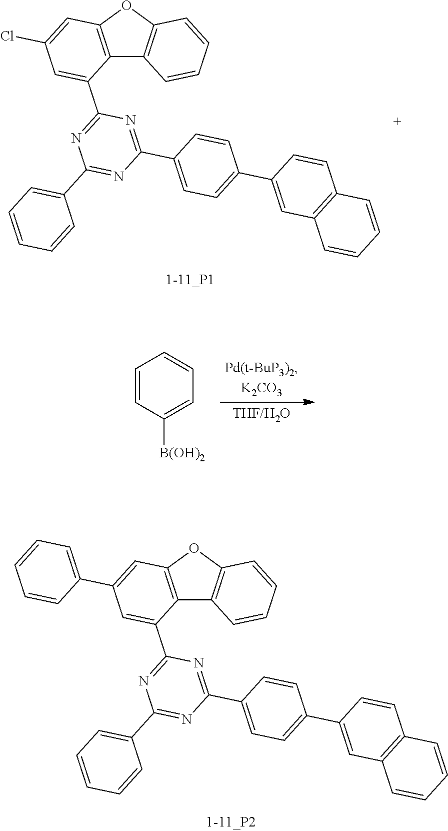 [SMILES: Clc1cc(-c2nc(-c3ccccc3)nc(-c3ccc(-c4ccc5ccccc5c4)cc3)n2)c2c(c1)oc1ccccc12 ; OB(O)c1ccccc1 ; c1ccc(-c2cc(-c3nc(-c4ccccc4)nc(-c4ccc(-c5ccc6ccccc6c5)cc4)n3)c3c(c2)oc2ccccc23)cc1]

Compound 1-11_P1 (15 g, 26.8 mmol) and phenylboronic acid (3.4 g, 28.1 mmol) were added to 300 ml of THF, and the mixture was stirred and refluxed. Then, potassium carbonate (11.1 g, 80.3 mmol) was dissolved in 33 ml of water and added thereto, and the mixture was sufficiently stirred and then bis(tri-tert-butylphosphine)palladium(0) (0.1 g, 0.3 mmol) was added. After the reaction for 2 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 11.8 g of Compound 1-11_P2. (Yield: 73%, MS: [M+H]⁺=602).

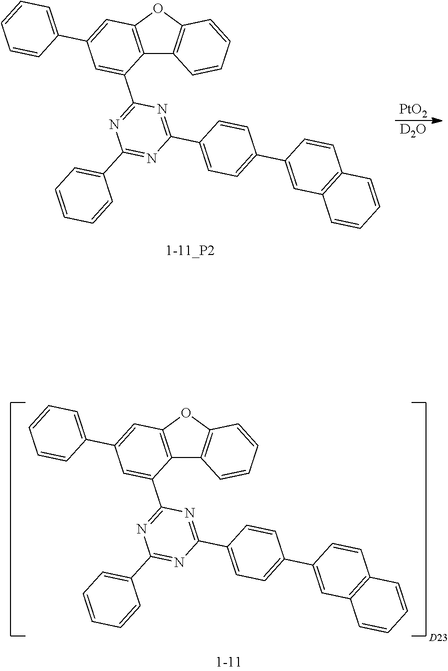

Compound 1-11_P2 (10 g, 16.6 mmol), PtO₂ (1.1 g, 5 mmol) and D₂O (83 ml) were added to a shaker tube, and then the tube was sealed and heated at 250° C. and 600 psi for 12 hours. When the reaction was completed, chloroform was added, and the reaction solution was transferred to a separatory funnel, and extracted. The extract was dried over MgSO₄ and concentrated, and then the sample was purified by silica gel column chromatography to prepare 3.1 g of Compound 1-11. (Yield: 30%, MS[M+H]+=626)

### Synthesis Example 1-12

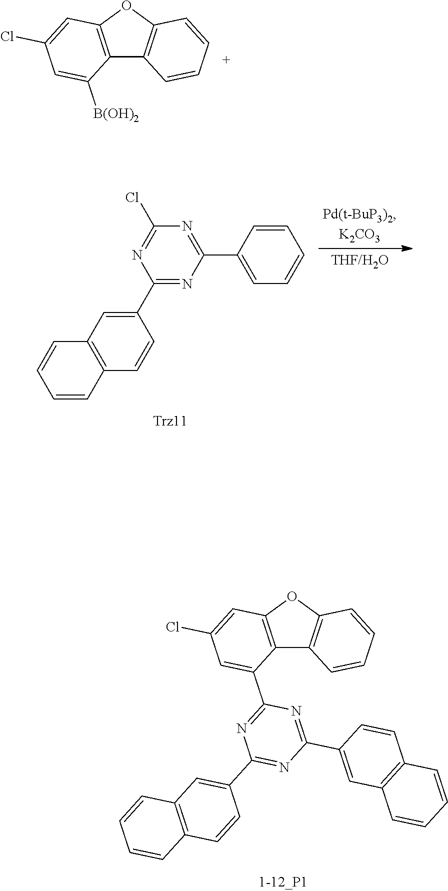 [SMILES: OB(O)c1cc(Cl)cc2oc3ccccc3c12 ; Clc1nc(-c2ccccc2)nc(-c2ccc3ccccc3c2)n1 ; Clc1cc(-c2nc(-c3ccc4ccccc4c3)nc(-c3ccc4ccccc4c3)n2)c2c(c1)oc1ccccc12]

(3-Chlorodibenzo[b,d]furan-1-yl)boronic acid (15 g, 60.9 mmol) and Compound Trz11 (23.5 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred and then bis(tri-tert-butylphosphine)palladium(0) (0.3 g, 0.6 mmol) was added. After the reaction for 2 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 23.4 g of Compound 1-12_P1. (Yield: 72%, MS: [M+H]⁺=534).

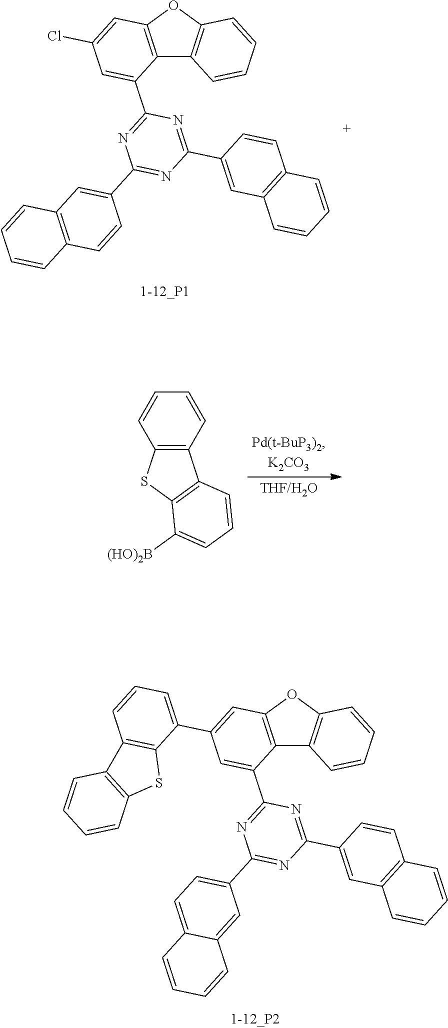 [SMILES: Clc1cc(-c2nc(-c3ccc4ccccc4c3)nc(-c3ccc4ccccc4c3)n2)c2c(c1)oc1ccccc12 ; OB(O)c1cccc2c1sc1ccccc12 ; c1ccc2cc(-c3nc(-c4ccc5ccccc5c4)nc(-c4cc(-c5cccc6c5sc5ccccc56)cc5oc6ccccc6c45)n3)ccc2c1]

Compound 1-12_P1 (15 g, 28.1 mmol) and dibenzo[b,d]thiophen-4-ylboronic acid (6.7 g, 29.5 mmol) were added to 300 ml of THF, and the mixture was stirred and refluxed. Then, potassium carbonate (11.6 g, 84.3 mmol) was dissolved in 35 ml of water and added thereto, and the mixture was sufficiently stirred and then bis(tri-tert-butylphosphine)palladium(0) (0.1 g, 0.3 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 12.4 g of Compound 1-12_P2. (Yield: 65%, MS: [M+H]⁺=682).

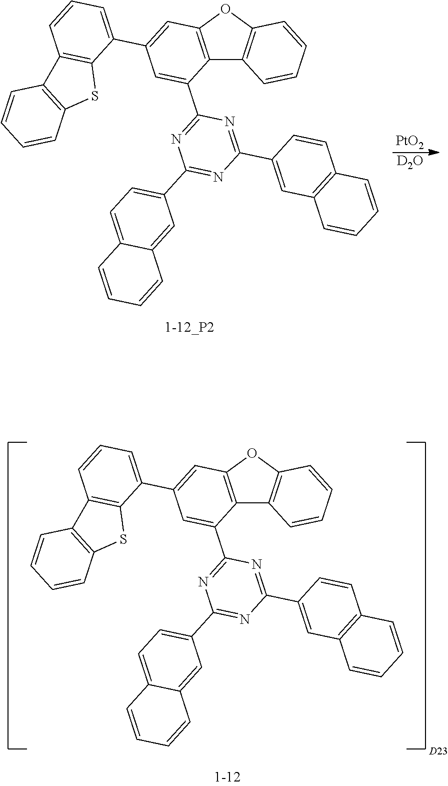

Compound 1-12_P2 (10 g, 14.7 mmol), PtO₂ (1 g, 4.4 mmol) and D₂O (73 ml) were added to a shaker tube, and then the tube was sealed and heated at 250° C. and 600 psi for 12 hours. When the reaction was completed, chloroform was added, and the reaction solution was transferred to a separatory funnel, and extracted. The extract was dried over MgSO₄ and concentrated, and then the sample was purified by silica gel column chromatography to prepare 5.1 g of Compound 1-12. (Yield: 49%, MS: [M+H]⁺=706)

### Synthesis Example 1-13

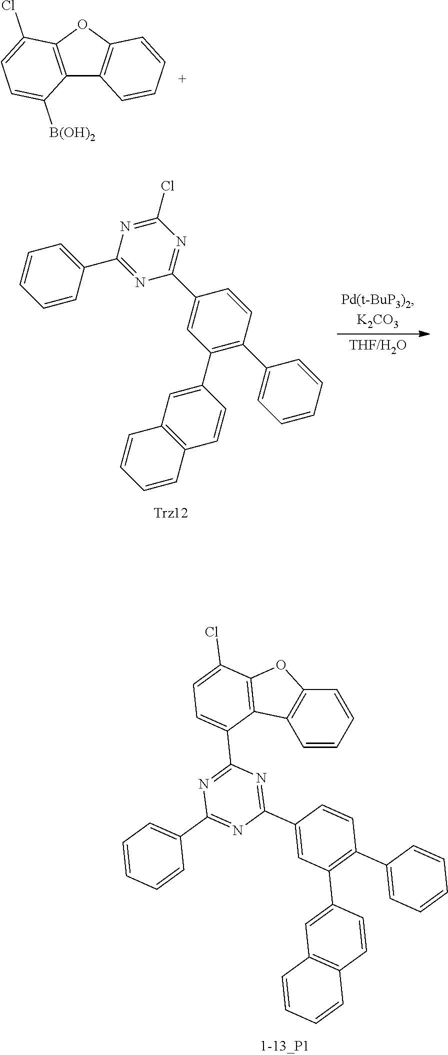 [SMILES: OB(O)c1ccc(Cl)c2oc3ccccc3c12 ; Clc1nc(-c2ccccc2)nc(-c2ccc(-c3ccccc3)c(-c3ccc4ccccc4c3)c2)n1 ; Clc1ccc(-c2nc(-c3ccccc3)nc(-c3ccc(-c4ccccc4)c(-c4ccc5ccccc5c4)c3)n2)c2c1oc1ccccc12]

(4-Chlorodibenzo[b,d]furan-1-yl)boronic acid (15 g, 60.9 mmol) and Compound Trz12 (30 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred and then bis(tri-tert-butylphosphine)palladium(0) (0.3 g, 0.6 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 24.4 g of Compound 1-13_P1. (Yield: 63%, MS: [M+H]⁺=636).

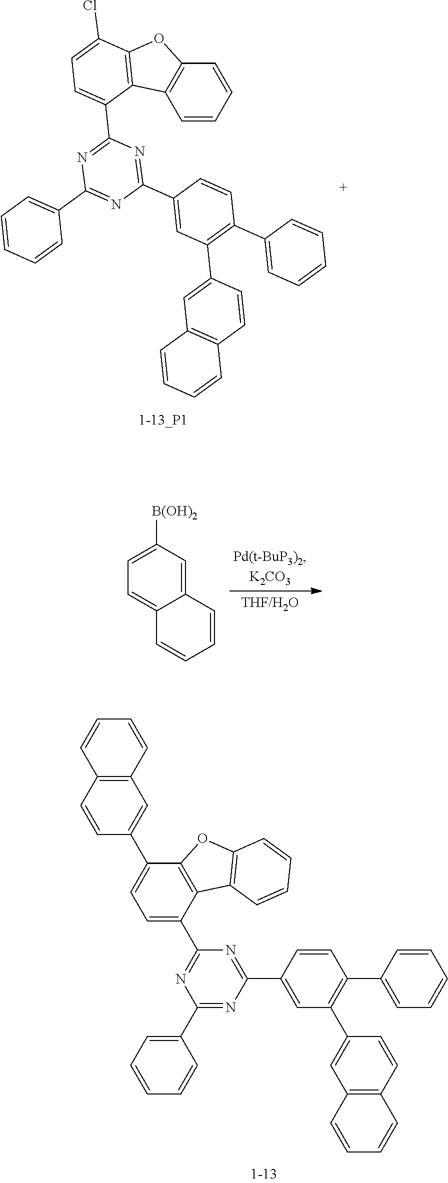 [SMILES: Clc1ccc(-c2nc(-c3ccccc3)nc(-c3ccc(-c4ccccc4)c(-c4ccc5ccccc5c4)c3)n2)c2c1oc1ccccc12 ; OB(O)c1ccc2ccccc2c1 ; c1ccc(-c2nc(-c3ccc(-c4ccccc4)c(-c4ccc5ccccc5c4)c3)nc(-c3ccc(-c4ccc5ccccc5c4)c4oc5ccccc5c34)n2)cc1]

Compound 1-13_P1 (15 g, 23.6 mmol) and naphthalen-2-ylboronic acid (4.3 g, 24.8 mmol) were added to 300 ml of THF, and the mixture was stirred and refluxed. Then, potassium carbonate (9.8 g, 70.7 mmol) was dissolved in 29 ml of water and added thereto, and the mixture was sufficiently stirred and then bis(tri-tert-butylphosphine)palladium(0) (0.1 g, 0.2 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 10.5 g of Compound 1-13. (Yield: 61%, MS: [M+H]⁺=728).

### Synthesis Example 1-14

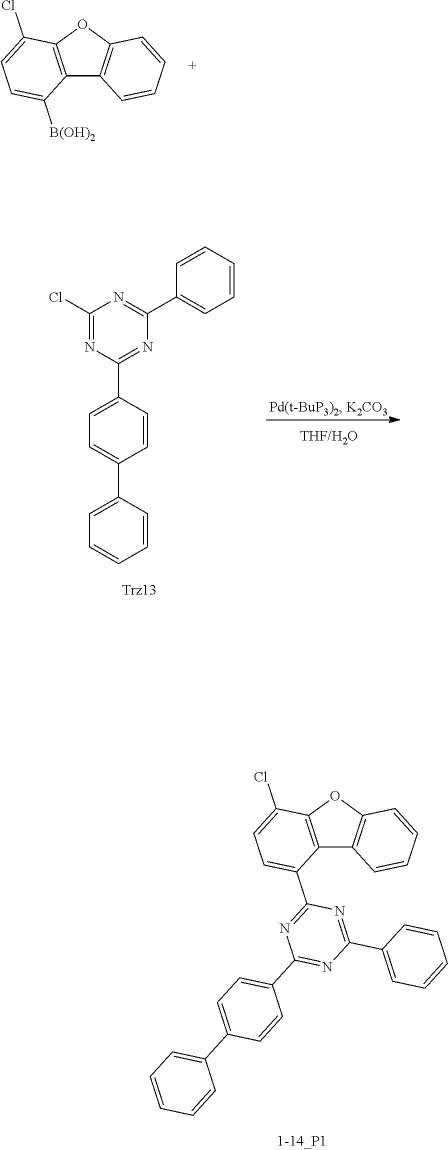 [SMILES: OB(O)c1ccc(Cl)c2oc3ccccc3c12 ; Clc1nc(-c2ccccc2)nc(-c2ccc(-c3ccccc3)cc2)n1 ; Clc1ccc(-c2nc(-c3ccccc3)nc(-c3ccc(-c4ccccc4)cc3)n2)c2c1oc1ccccc12]

(4-Chlorodibenzo[b,d]furan-1-yl)boronic acid (15 g, 60.9 mmol) and Compound Trz13 (22 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred and then bis(tri-tert-butylphosphine)palladium(0) (0.3 g, 0.6 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 22.3 g of Compound 1-14_P1. (Yield: 72%, MS: [M+H]⁺=510).

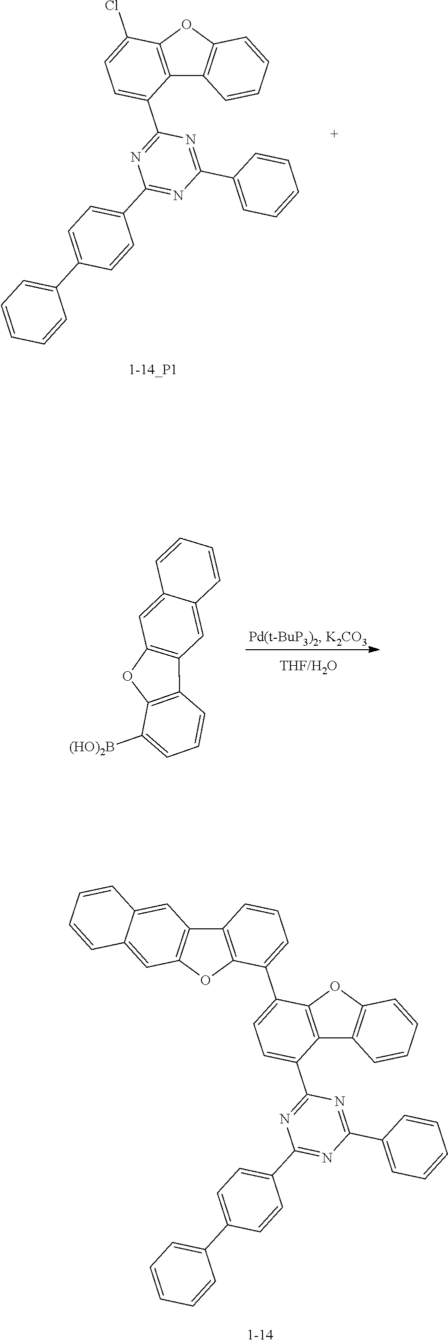 [SMILES: Clc1ccc(-c2nc(-c3ccccc3)nc(-c3ccc(-c4ccccc4)cc3)n2)c2c1oc1ccccc12 ; OB(O)c1cccc2c1oc1cc3ccccc3cc12 ; c1ccc(-c2ccc(-c3nc(-c4ccccc4)nc(-c4ccc(-c5cccc6c5oc5cc7ccccc7cc56)c5oc6ccccc6c45)n3)cc2)cc1]

Compound 1-14_P1 (15 g, 29.4 mmol) and naphtho[2,3-b]benzofuran-4-ylboronic acid (8.1 g, 30.9 mmol) were added to 300 ml of THF, and the mixture was stirred and refluxed. Then, potassium carbonate (12.2 g, 88.2 mmol) was dissolved in 37 ml of water and added thereto, and the mixture was sufficiently stirred and then bis(tri-tert-butylphosphine)palladium(0) (0.2 g, 0.3 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 14.2 g of Compound 1-14. (Yield: 70%, MS: [M+H]⁺=692).

### Synthesis Example 1-15

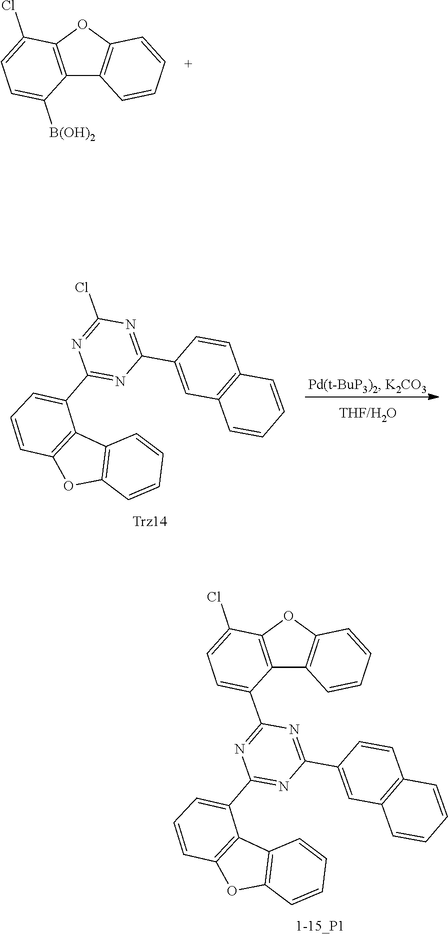 [SMILES: OB(O)c1ccc(Cl)c2oc3ccccc3c12 ; Clc1nc(-c2ccc3ccccc3c2)nc(-c2cccc3oc4ccccc4c23)n1 ; Clc1ccc(-c2nc(-c3ccc4ccccc4c3)nc(-c3cccc4oc5ccccc5c34)n2)c2c1oc1ccccc12]

(4-Chlorodibenzo[b,d]furan-1-yl)boronic acid (15 g, 60.9 mmol) and Compound Trz14 (26.1 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred and then bis(tri-tert-butylphosphine)palladium(0) (0.3 g, 0.6 mmol) was added. After the reaction for 2 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 24.8 g of Compound 1-15_P1. (Yield: 71%, MS: [M+H]⁺=574).

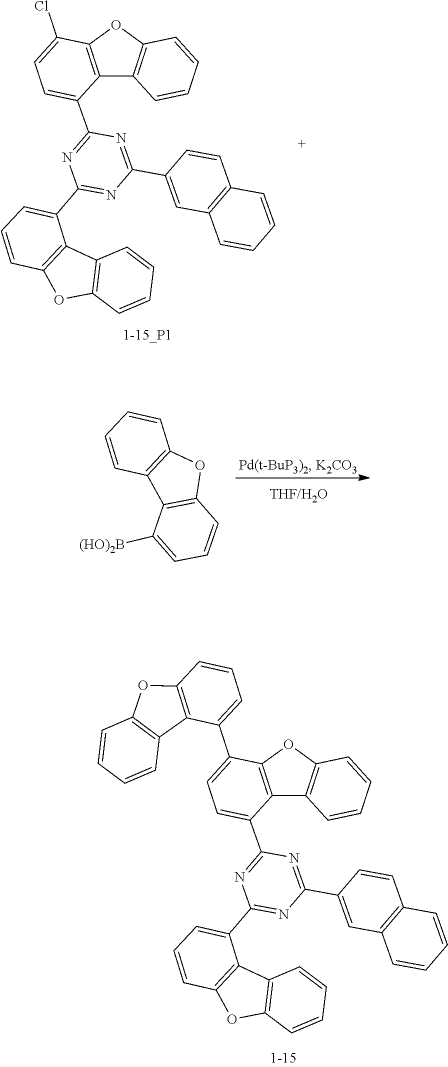 [SMILES: Clc1ccc(-c2nc(-c3ccc4ccccc4c3)nc(-c3cccc4oc5ccccc5c34)n2)c2c1oc1ccccc12 ; OB(O)c1cccc2oc3ccccc3c12 ; c1ccc2cc(-c3nc(-c4cccc5oc6ccccc6c45)nc(-c4ccc(-c5cccc6oc7ccccc7c56)c5oc6ccccc6c45)n3)ccc2c1]

Compound 1-15_P1 (15 g, 26.1 mmol) and dibenzo[b,d]furan-1-ylboronic acid (5.8 g, 27.4 mmol) were added to 300 ml of THF, and the mixture was stirred and refluxed. Then, potassium carbonate (10.8 g, 78.4 mmol) was dissolved in 33 ml of water and added thereto, and the mixture was sufficiently stirred and then bis(tri-tert-butylphosphine)palladium(0) (0.1 g, 0.3 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 11.1 g of Compound 1-15. (Yield: 60%, MS: [M+H]⁺=706).

### Synthesis Example 1-16

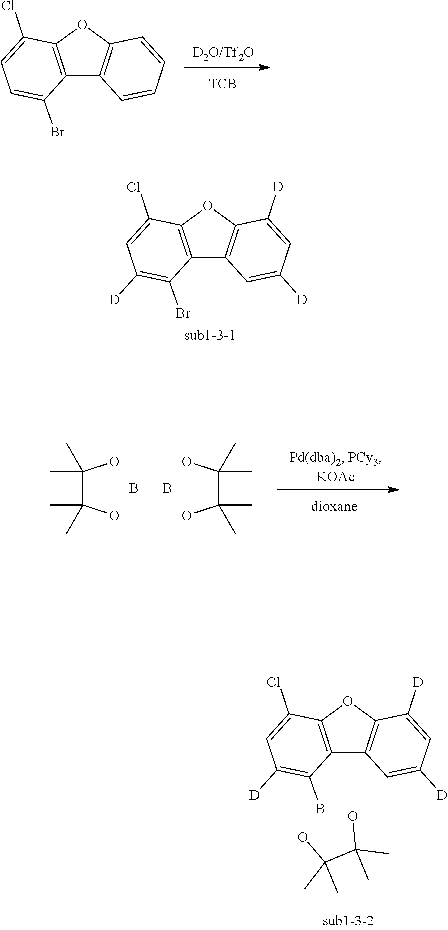

Trifluoromethanesulfonic anhydride (45.1 g, 159.8 mmol) and deuterium oxide (16 g, 799.2 mmol) were added at 0° C. and stirred for 5 hours to prepare a solution. 1-Bromo-4-chlorodibenzo[b,d]furan (15 g, 53.3 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of 1-bromo-4-chlorodibenzo[b,d]furan and 1,2,4-trichlorobenzene, and the mixture was stirred while heating up to 140° C. and then keeping that temperature. After the reaction for 7 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 5.6 g of compound Sub1-3-1. (Yield: 37%, MS: [M+H]⁺=284)

Compound Sub1-3-1 (15 g, 52.7 mmol) and bis(pinacolato)diboron (14.7 g, 58 mmol) were added to 300 ml of 1,4-dioxane, and the mixture was stirred under reflux. Then, potassium acetate (7.8 g, 79.1 mmol) was added thereto, the mixture was sufficiently stirred, and then bis(dibenzylideneacetone) palladium(0) (0.9 g, 1.6 mmol) and tricyclohexylphosphine (0.9 g, 3.2 mmol) were added. After the reaction for 6 hours, the reaction mixture was cooled to room temperature, the organic layer was separated using chloroform and water, and then the organic layer was distilled. This was dissolved again in chloroform, washed twice with water, and the organic layer was then separated. Anhydrous magnesium sulfate was added thereto, stirred, then filtered, and the filtrate was distilled under reduced pressure. The concentrated compound was purified by silica gel column chromatography to prepare 10.1 g of Compound Sub1-3-2. (Yield: 58%, MS: [M+H]⁺=332)

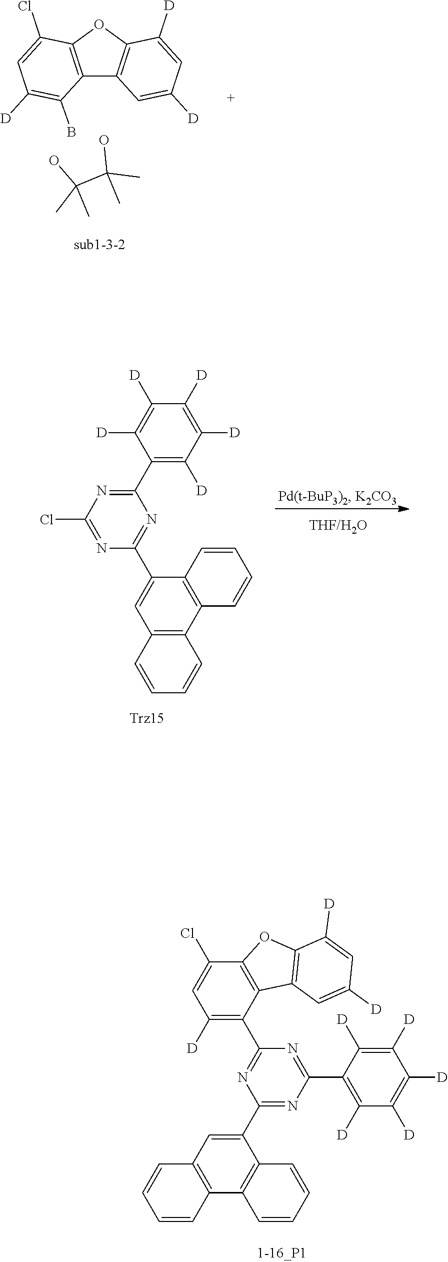

Compound Sub1-3-2 (15 g, 45.2 mmol) and Compound Trz15 (17.7 g, 47.5 mmol) were added to 300 ml of THF, and the mixture was stirred and refluxed. Then, potassium carbonate (18.8 g, 135.7 mmol) was dissolved in 56 ml of water and added thereto, and the mixture was sufficiently stirred and then bis(tri-tert-butylphosphine)palladium(0) (0.2 g, 0.5 mmol) was added. After the reaction for 2 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 15.4 g of Compound 1-16_P1. (Yield: 63%, MS: [M+H]⁺=542).

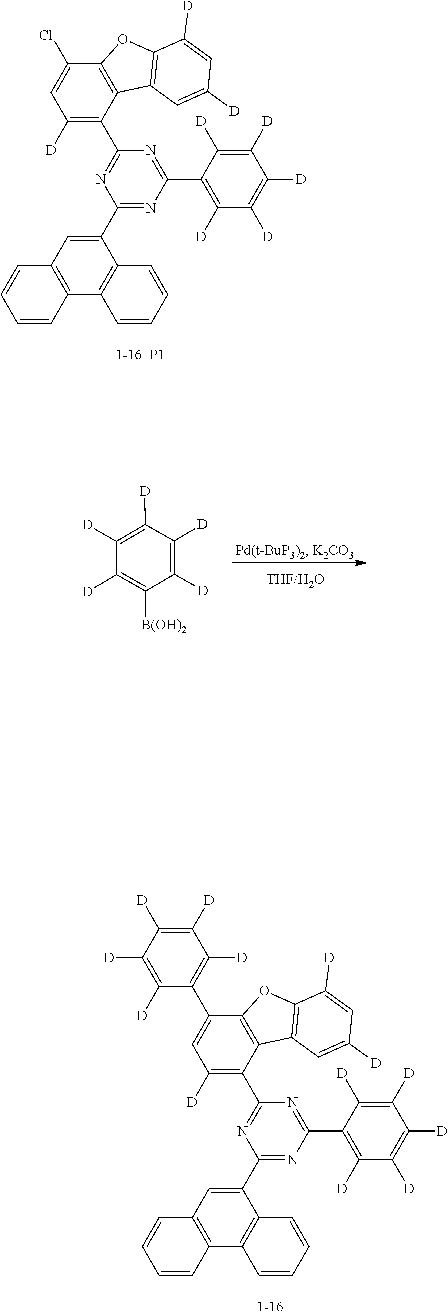 [SMILES: [2H]c1cc([2H])c2oc3c(Cl)cc([2H])c(-c4nc(-c5c([2H])c([2H])c([2H])c([2H])c5[2H])nc(-c5cc6ccccc6c6ccccc56)n4)c3c2c1 ; [2H]c1c([2H])c([2H])c(B(O)O)c([2H])c1[2H] ; [2H]c1cc([2H])c2oc3c(-c4c([2H])c([2H])c([2H])c([2H])c4[2H])cc([2H])c(-c4nc(-c5c([2H])c([2H])c([2H])c([2H])c5[2H])nc(-c5cc6ccccc6c6ccccc56)n4)c3c2c1]

Compound 1-16_P1 (15 g, 27.7 mmol) and (phenyl-d5)boronic acid (3.7 g, 29.1 mmol) were added to 300 ml of THF, and the mixture was stirred and refluxed. Then, potassium carbonate (11.5 g, 83 mmol) was dissolved in 34 ml of water and added thereto, and the mixture was sufficiently stirred and then bis(tri-tert-butylphosphine)palladium(0) (0.1 g, 0.3 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 11.9 g of Compound 1-16. (Yield: 73%, MS: [M+H]⁺=589).

### Synthesis Example 1-17

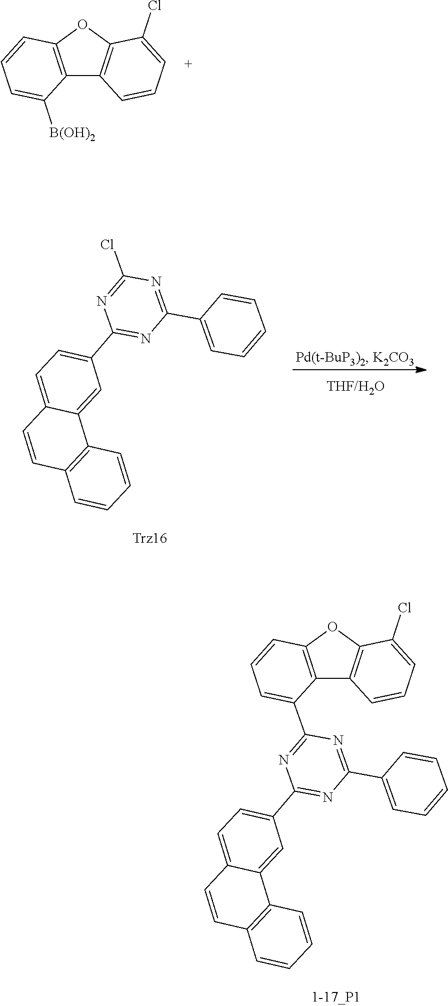 [SMILES: OB(O)c1cccc2oc3c(Cl)cccc3c12 ; Clc1nc(-c2ccccc2)nc(-c2ccc3ccc4ccccc4c3c2)n1 ; Clc1cccc2c1oc1cccc(-c3nc(-c4ccccc4)nc(-c4ccc5ccc6ccccc6c5c4)n3)c12]

(6-Chlorodibenzo[b,d]furan-1-yl)boronic acid (15 g, 60.9 mmol) and Compound Trz16 (23.5 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred and then bis(tri-tert-butylphosphine)palladium(0) (0.3 g, 0.6 mmol) was added. After the reaction for 2 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 23.4 g of Compound 1-17_P1. (Yield: 72%, MS: [M+H]⁺=534).

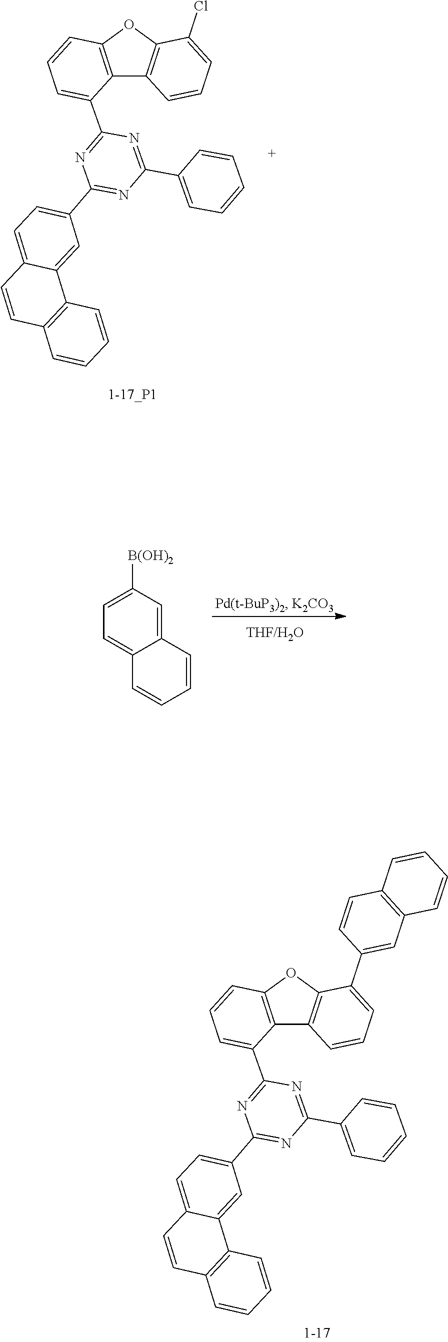 [SMILES: Clc1cccc2c1oc1cccc(-c3nc(-c4ccccc4)nc(-c4ccc5ccc6ccccc6c5c4)n3)c12 ; OB(O)c1ccc2ccccc2c1 ; c1ccc(-c2nc(-c3ccc4ccc5ccccc5c4c3)nc(-c3cccc4oc5c(-c6ccc7ccccc7c6)cccc5c34)n2)cc1]

Compound 1-17_P1 (15 g, 28.1 mmol) and naphthalen-2-ylboronic acid (5.1 g, 29.5 mmol) were added to 300 ml of THF, and the mixture was stirred and refluxed. Then, potassium carbonate (11.6 g, 84.3 mmol) was dissolved in 35 ml of water and added thereto, and the mixture was sufficiently stirred and then bis(tri-tert-butylphosphine)palladium(0) (0.1 g, 0.3 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 12.5 g of Compound 1-17. (Yield: 71%, MS: [M+H]⁺=626).

### Synthesis Example 1-18

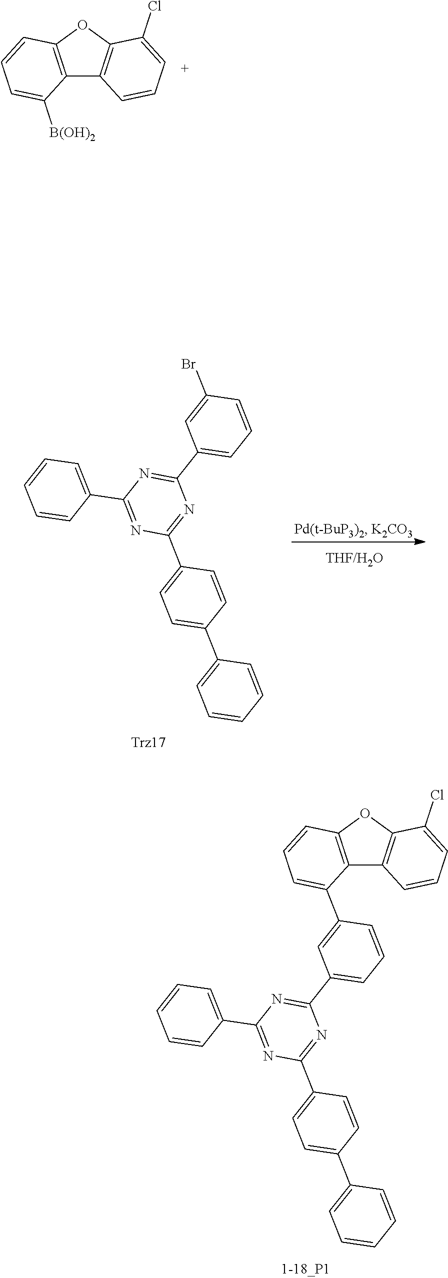 [SMILES: OB(O)c1cccc2oc3c(Cl)cccc3c12 ; Brc1cccc(-c2nc(-c3ccccc3)nc(-c3ccc(-c4ccccc4)cc3)n2)c1 ; Clc1cccc2c1oc1cccc(-c3cccc(-c4nc(-c5ccccc5)nc(-c5ccc(-c6ccccc6)cc5)n4)c3)c12]

(6-Chlorodibenzo[b,d]furan-1-yl)boronic acid (15 g, 60.9 mmol) and Compound Trz17 (29.7 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred and then bis(tri-tert-butylphosphine)palladium(0) (0.3 g, 0.6 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 24.6 g of Compound 1-18_P1. (Yield: 69%, MS: [M+H]⁺=586).

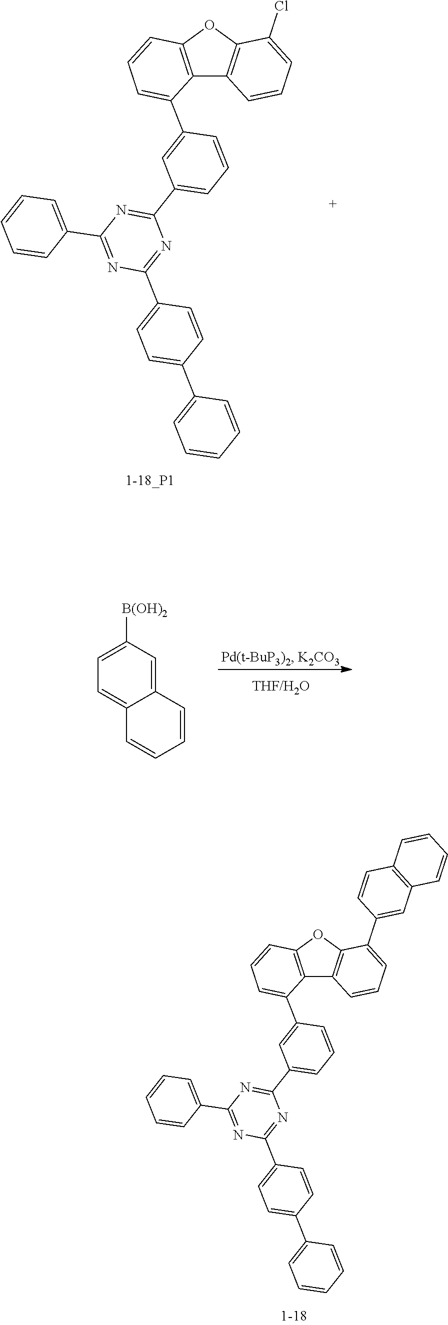 [SMILES: Clc1cccc2c1oc1cccc(-c3cccc(-c4nc(-c5ccccc5)nc(-c5ccc(-c6ccccc6)cc5)n4)c3)c12 ; OB(O)c1ccc2ccccc2c1 ; c1ccc(-c2ccc(-c3nc(-c4ccccc4)nc(-c4cccc(-c5cccc6oc7c(-c8ccc9ccccc9c8)cccc7c56)c4)n3)cc2)cc1]

Compound 1-18_P1 (15 g, 25.6 mmol) and naphthalen-2-ylboronic acid (4.6 g, 26.9 mmol) were added to 300 ml of THF, and the mixture was stirred and refluxed. Then, potassium carbonate (10.6 g, 76.8 mmol) was dissolved in 32 ml of water and added thereto, and the mixture was sufficiently stirred and then bis(tri-tert-butylphosphine)palladium(0) (0.1 g, 0.3 mmol) was added. After the reaction for 2 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 10.9 g of Compound 1-18. (Yield: 63%, MS: [M+H]⁺=678).

### Synthesis Example 1-19

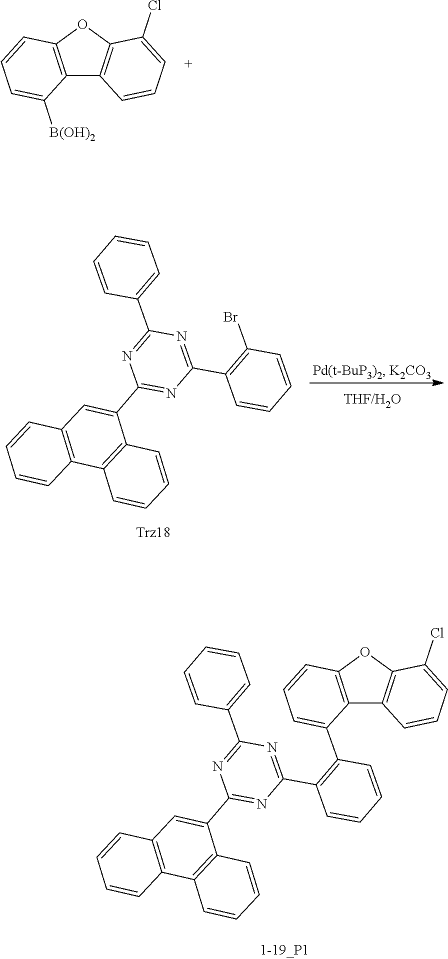 [SMILES: OB(O)c1cccc2oc3c(Cl)cccc3c12 ; Brc1ccccc1-c1nc(-c2ccccc2)nc(-c2cc3ccccc3c3ccccc23)n1 ; Clc1cccc2c1oc1cccc(-c3ccccc3-c3nc(-c4ccccc4)nc(-c4cc5ccccc5c5ccccc45)n3)c12]

(6-Chlorodibenzo[b,d]furan-1-yl)boronic acid (15 g, 60.9 mmol) and Compound Trz18 (31.2 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred and then bis(tri-tert-butylphosphine)palladium(0) (0.3 g, 0.6 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 24.5 g of Compound 1-19_P1. (Yield: 66%, MS: [M+H]⁺=610).

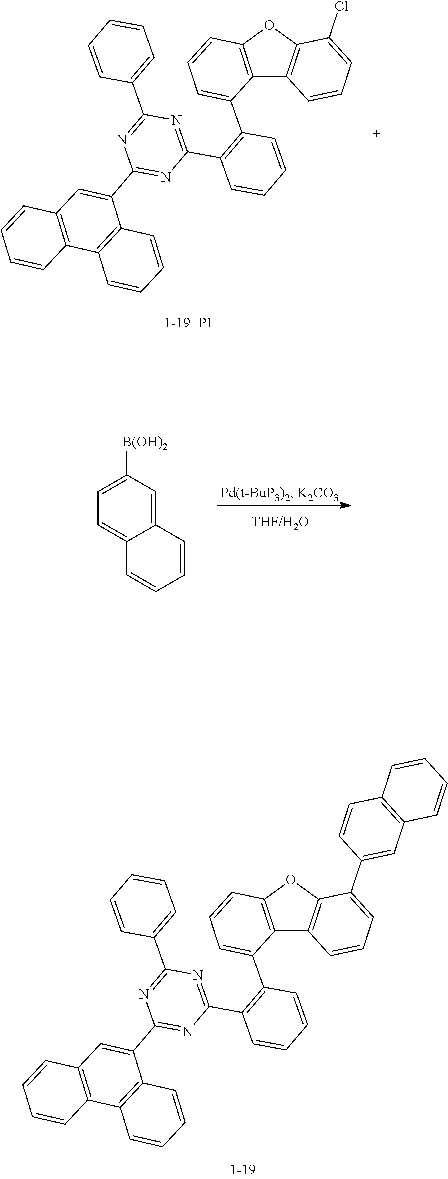 [SMILES: Clc1cccc2c1oc1cccc(-c3ccccc3-c3nc(-c4ccccc4)nc(-c4cc5ccccc5c5ccccc45)n3)c12 ; OB(O)c1ccc2ccccc2c1 ; c1ccc(-c2nc(-c3ccccc3-c3cccc4oc5c(-c6ccc7ccccc7c6)cccc5c34)nc(-c3cc4ccccc4c4ccccc34)n2)cc1]

Compound 1-19_P1 (15 g, 24.6 mmol) and naphthalen-2-ylboronic acid (4.4 g, 25.8 mmol) were added to 300 ml of THF, and the mixture was stirred and refluxed. Then, potassium carbonate (10.2 g, 73.8 mmol) was dissolved in 31 ml of water and added thereto, and the mixture was sufficiently stirred and then bis(tri-tert-butylphosphine)palladium(0) (0.1 g, 0.2 mmol) was added. After the reaction for 2 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 11.4 g of Compound 1-19. (Yield: 66%, MS: [M+H]⁺=702).

### Synthesis Example 1-20

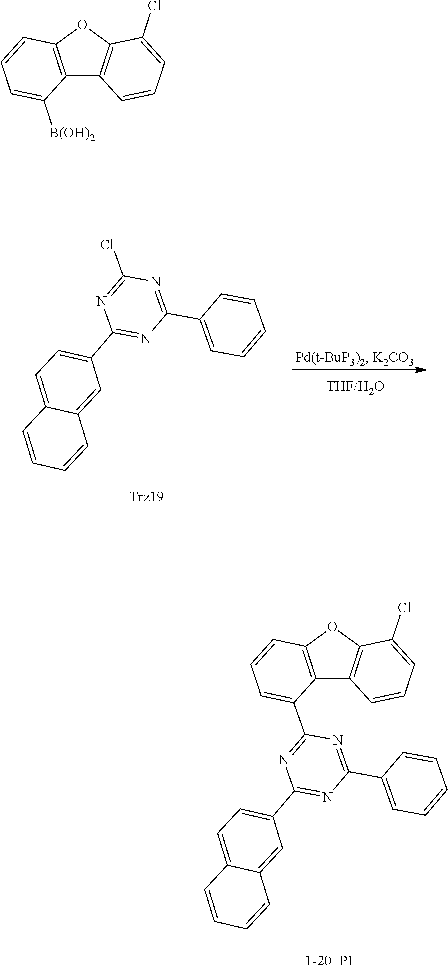 [SMILES: OB(O)c1cccc2oc3c(Cl)cccc3c12 ; Clc1nc(-c2ccccc2)nc(-c2ccc3ccccc3c2)n1 ; Clc1cccc2c1oc1cccc(-c3nc(-c4ccccc4)nc(-c4ccc5ccccc5c4)n3)c12]

(6-Chlorodibenzo[b,d]furan-1-yl)boronic acid (15 g, 60.9 mmol) and Compound Trz19 (20.3 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred and then bis(tri-tert-butylphosphine)palladium(0) (0.3 g, 0.6 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 19.1 g of Compound 1-20_P1. (Yield: 65%, MS: [M+H]⁺=484).

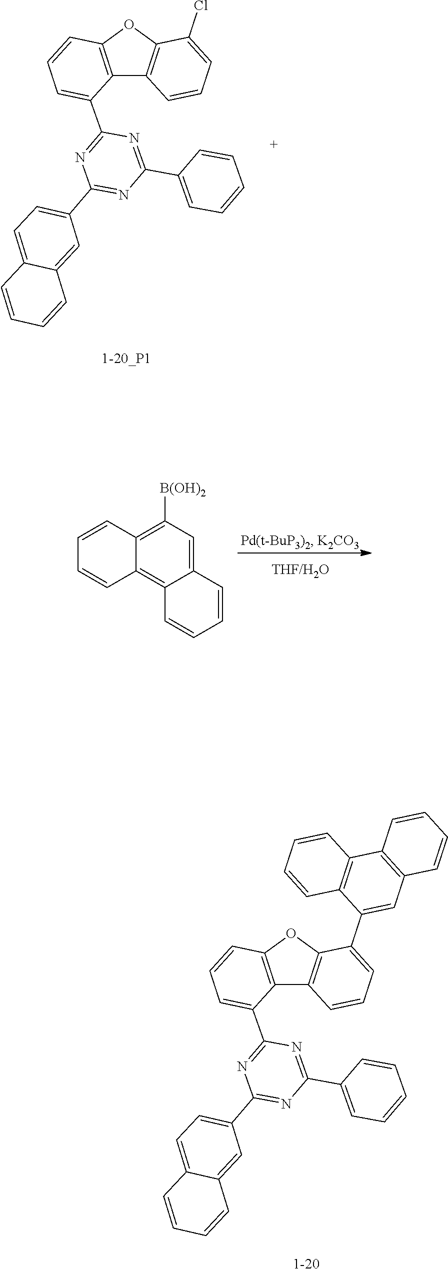 [SMILES: Clc1cccc2c1oc1cccc(-c3nc(-c4ccccc4)nc(-c4ccc5ccccc5c4)n3)c12 ; OB(O)c1cc2ccccc2c2ccccc12 ; c1ccc(-c2nc(-c3ccc4ccccc4c3)nc(-c3cccc4oc5c(-c6cc7ccccc7c7ccccc67)cccc5c34)n2)cc1]

Compound 1-20_P1 (15 g, 31 mmol) and phenanthren-9-ylboronic acid (7.2 g, 32.5 mmol) were added to 300 ml of THF, and the mixture was stirred and refluxed. Then, potassium carbonate (12.9 g, 93 mmol) was dissolved in 39 ml of water and added thereto, and the mixture was sufficiently stirred and then bis(tri-tert-butylphosphine)palladium(0) (0.2 g, 0.3 mmol) was added. After the reaction for 2 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 12 g of Compound 1-20. (Yield: 62%, MS: [M+H]⁺=626).

### Synthesis Example 1-21

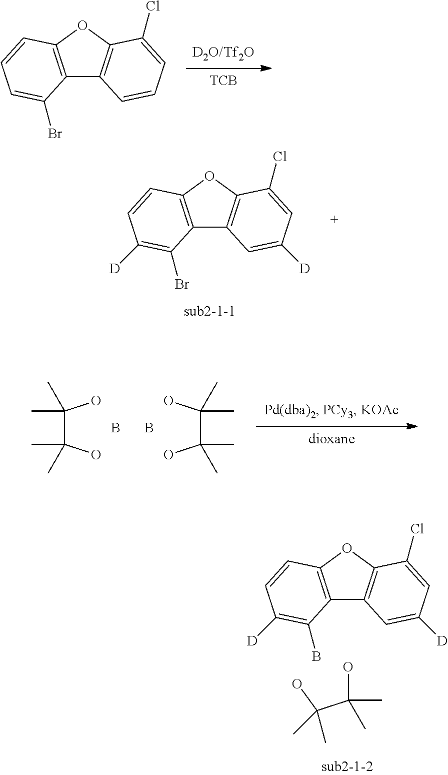

Trifluoromethanesulfonic anhydride (30.1 g, 106.6 mmol) and deuterium oxide (10.7 g, 532.8 mmol) were added at 0° C. and stirred for 5 hours to prepare a solution. 1-Bromo-6-chlorodibenzo[b,d]furan (15 g, 53.3 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of 1-bromo-6-chlorodibenzo[b,d]furan and 1,2,4-trichlorobenzene, and the mixture was stirred while heating up to 140° C. and then keeping that temperature. After the reaction for 3 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 6.8 g of compound Sub2-1-1. (Yield: 45%, MS: [M+H]⁺=283)

Compound Sub2-1-1 (15 g, 52.9 mmol) and bis(pinacolato)diboron (14.8 g, 58.2 mmol) were added to 300 ml of 1,4-dioxane, and the mixture was stirred under reflux. Then, potassium acetate (7.8 g, 79.4 mmol) was added thereto, the mixture was sufficiently stirred, and then bis(dibenzylideneacetone) palladium(0) (0.9 g, 1.6 mmol) and tricyclohexylphosphine (0.9 g, 3.2 mmol) were added. After the reaction for 6 hours, the reaction mixture was cooled to room temperature, the organic layer was separated using chloroform and water, and then the organic layer was distilled. This was dissolved again in chloroform, washed twice with water, and the organic layer was then separated. Anhydrous magnesium sulfate was added thereto, stirred, then filtered, and the filtrate was distilled under reduced pressure. The concentrated compound was purified by silica gel column chromatography to prepare 13.1 g of Compound Sub2-1-2. (Yield: 75%, MS: [M+H]⁺=331)

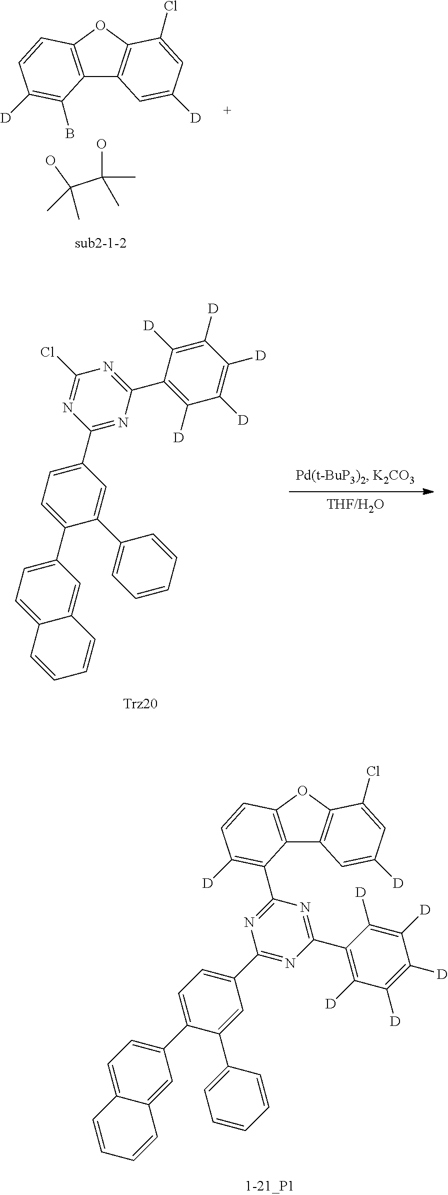

Compound Sub2-1-2 (15 g, 45.4 mmol) and Compound Trz20 (22.6 g, 47.6 mmol) were added to 300 ml of THF, and the mixture was stirred and refluxed. Then, potassium carbonate (18.8 g, 136.1 mmol) was dissolved in 56 ml of water and added thereto, and the mixture was sufficiently stirred and then bis(tri-tert-butylphosphine)palladium(0) (0.2 g, 0.5 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 17.8 g of Compound 1-21_P1. (Yield: 61%, MS: [M+H]⁺=643).

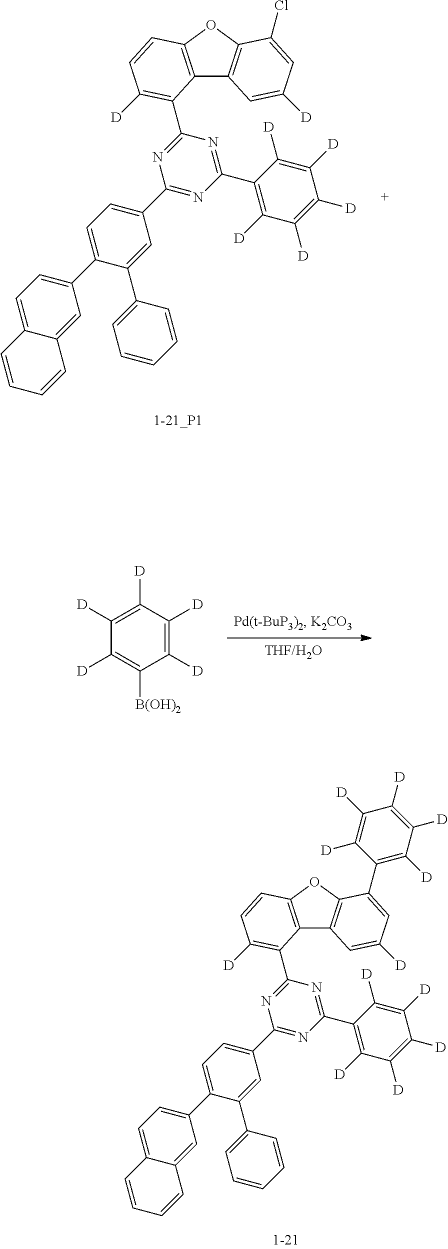 [SMILES: [2H]c1cc(Cl)c2oc3ccc([2H])c(-c4nc(-c5ccc(-c6ccc7ccccc7c6)c(-c6ccccc6)c5)nc(-c5c([2H])c([2H])c([2H])c([2H])c5[2H])n4)c3c2c1 ; [2H]c1c([2H])c([2H])c(B(O)O)c([2H])c1[2H] ; "THF/H2O" ; [2H]c1cc(-c2c([2H])c([2H])c([2H])c([2H])c2[2H])c2oc3ccc([2H])c(-c4nc(-c5ccc(-c6ccc7ccccc7c6)c(-c6ccccc6)c5)nc(-c5c([2H])c([2H])c([2H])c([2H])c5[2H])n4)c3c2c1]

Compound 1-21_P1 (15 g, 23.3 mmol) and (phenyl-d5)boronic acid (3.1 g, 24.5 mmol) were added to 300 ml of THF, and the mixture was stirred and refluxed. Then, potassium carbonate (9.7 g, 70 mmol) was dissolved in 29 ml of water and added thereto, and the mixture was sufficiently stirred and then bis(tri-tert-butylphosphine)palladium(0) (0.1 g, 0.2 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 11.9 g of Compound 1-21. (Yield: 74%, MS: [M+H]⁺=690).

### Synthesis Example 1-22

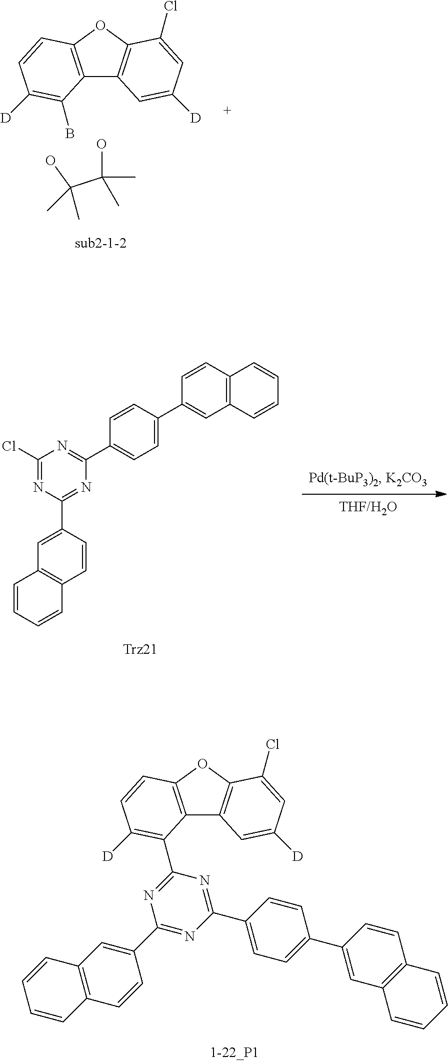

Compound Sub2-1-2 (15 g, 45.4 mmol) and Compound Trz21 (21.1 g, 47.6 mmol) were added to 300 ml of THF, and the mixture was stirred and refluxed. Then, potassium carbonate (18.8 g, 136.1 mmol) was dissolved in 56 ml of water and added thereto, and the mixture was sufficiently stirred and then bis(tri-tert-butylphosphine)palladium(0) (0.2 g, 0.5 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 18.6 g of Compound 1-22_P1. (Yield: 67%, MS: [M+H]⁺=612).

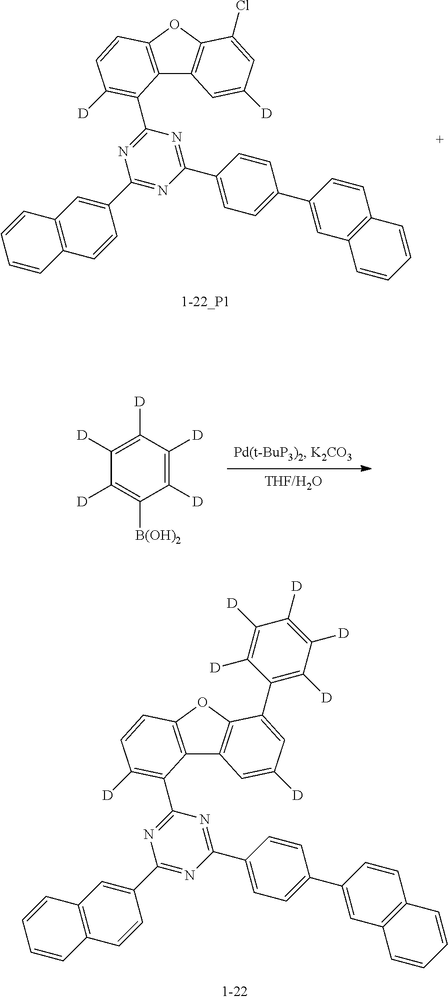 [SMILES: [2H]c1cc(Cl)c2oc3ccc([2H])c(-c4nc(-c5ccc(-c6ccc7ccccc7c6)cc5)nc(-c5ccc6ccccc6c5)n4)c3c2c1 ; [2H]c1c([2H])c([2H])c(B(O)O)c([2H])c1[2H] ; "THF/H2O" ; [2H]c1cc(-c2c([2H])c([2H])c([2H])c([2H])c2[2H])c2oc3ccc([2H])c(-c4nc(-c5ccc(-c6ccc7ccccc7c6)cc5)nc(-c5ccc6ccccc6c5)n4)c3c2c1]

Compound 1-22_P1 (15 g, 24.5 mmol) and (phenyl-d5)boronic acid (3.3 g, 25.7 mmol) were added to 300 ml of THF, and the mixture was stirred and refluxed. Then, potassium carbonate (10.2 g, 73.5 mmol) was dissolved in 30 ml of water and added thereto, and the mixture was sufficiently stirred and then bis(tri-tert-butylphosphine)palladium(0) (0.1 g, 0.2 mmol) was added. After the reaction for 2 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 10 g of Compound 1-22. (Yield: 62%, MS: [M+H]⁺=659).

### Synthesis Example 1-23

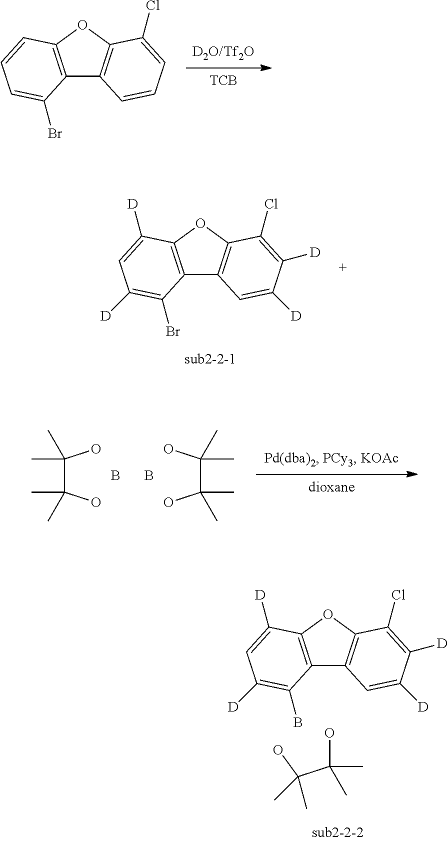

Trifluoromethanesulfonic anhydride (60.1 g, 213.1 mmol) and deuterium oxide (21.4 g, 1065.6 mmol) were added at 0° C. and stirred for hours to prepare a solution. 1-Bromo-6-chlorodibenzo[b,d]furan (15 g, 53.3 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of 1-bromo-6-chlorodibenzo[b,d]furan and 1,2,4-trichlorobenzene, and the mixture was stirred while heating up to 140° C. and then keeping that temperature. After the reaction for 10 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 6.5 g of compound Sub2-2-1. (Yield: 43%, MS: [M+H]⁺=285)

Compound Sub2-2-1 (15 g, 52.5 mmol) and bis(pinacolato)diboron (14.7 g, 57.8 mmol) were added to 300 ml of 1,4-dioxane, and the mixture was stirred under reflux. Then, potassium acetate (7.7 g, 78.8 mmol) was added thereto, the mixture was sufficiently stirred, and then bis(dibenzylideneacetone) palladium(0) (0.9 g, 1.6 mmol) and tricyclohexylphosphine (0.9 g, 3.2 mmol) were added. After the reaction for 5 hours, the reaction mixture was cooled to room temperature, the organic layer was separated using chloroform and water, and then the organic layer was distilled. This was dissolved again in chloroform, washed twice with water, and the organic layer was then separated. Anhydrous magnesium sulfate was added thereto, stirred, then filtered, and the filtrate was distilled under reduced pressure. The concentrated compound was purified by silica gel column chromatography to prepare 13.1 g of Compound Sub2-2-2. (Yield: 75%, MS: [M+H]⁺=333)

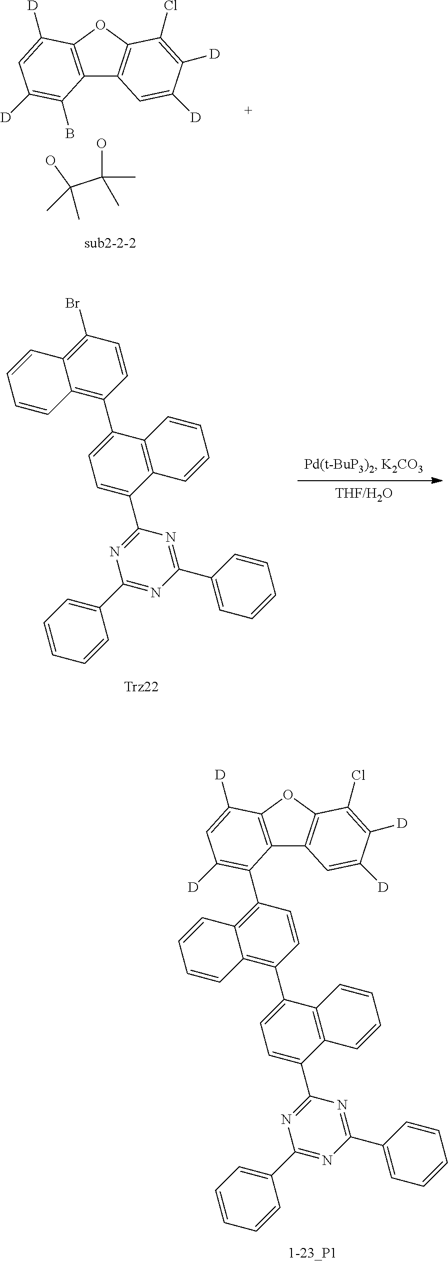

Compound Sub2-2-2 (15 g, 60.9 mmol) and Compound Trz22 (36.1 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred and then bis(tri-tert-butylphosphine)palladium(0) (0.3 g, 0.6 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 28.9 g of Compound 1-23_P1. (Yield: 69%, MS: [M+H]⁺=690).

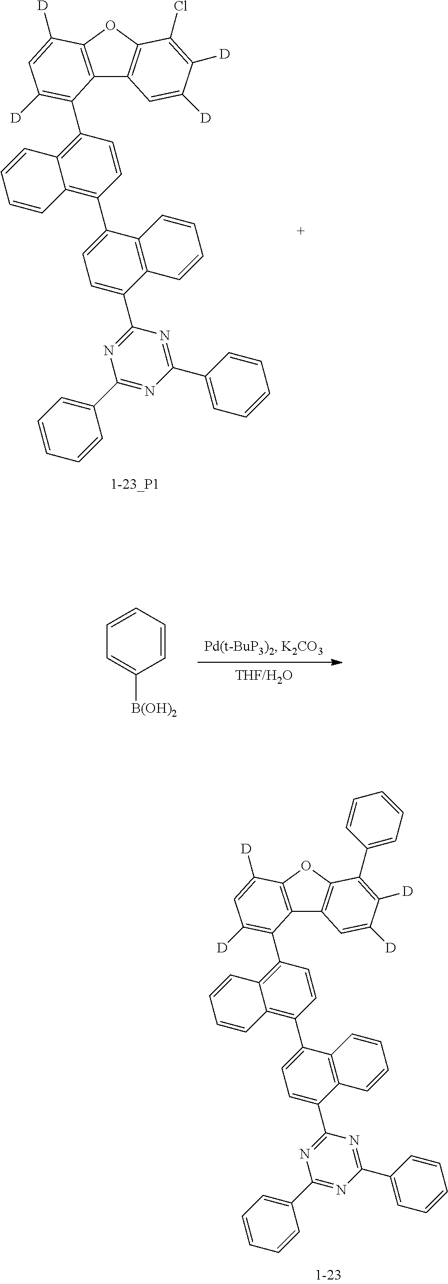 [SMILES: [2H]c1cc2c(oc3c([2H])cc([2H])c(-c4ccc(-c5ccc(-c6nc(-c7ccccc7)nc(-c7ccccc7)n6)c6ccccc56)c5ccccc45)c32)c(Cl)c1[2H] ; OB(O)c1ccccc1 ; "THF/H2O" ; [2H]c1cc2c(oc3c([2H])cc([2H])c(-c4ccc(-c5ccc(-c6nc(-c7ccccc7)nc(-c7ccccc7)n6)c6ccccc56)c5ccccc45)c32)c(-c2ccccc2)c1[2H]]

Compound 1-23_P1 (15 g, 21.7 mmol) and phenylboronic acid (2.8 g, 22.8 mmol) were added to 300 ml of THF, and the mixture was stirred and refluxed. Then, potassium carbonate (9 g, 65.2 mmol) was dissolved in 27 ml of water and added thereto, and the mixture was sufficiently stirred and then bis(tri-tert-butylphosphine)palladium(0) (0.1 g, 0.2 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 9.5 g of Compound 1-23. (Yield: 60%, MS: [M+H]⁺=732).

### Synthesis Example 1-24

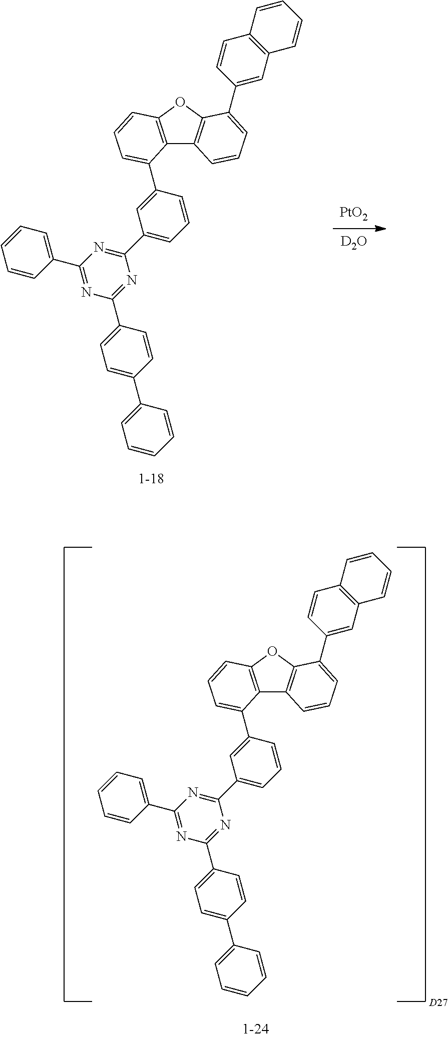

Compound 1-18 (10 g, 14.8 mmol), PtO₂ (1 g, 4.4 mmol) and D₂O (74 ml) were added to a shaker tube, and then the tube was sealed and heated at 250° C. and 600 psi for 12 hours. When the reaction was completed, chloroform was added, and the reaction solution was transferred to a separatory funnel, and extracted. The extract was dried over MgSO₄ and concentrated, and then the sample was purified by silica gel column chromatography to prepare 5.1 g of Compound 1-24. (Yield: 49%, MS: [M+H]⁺=706)

### Synthesis Example 1-25

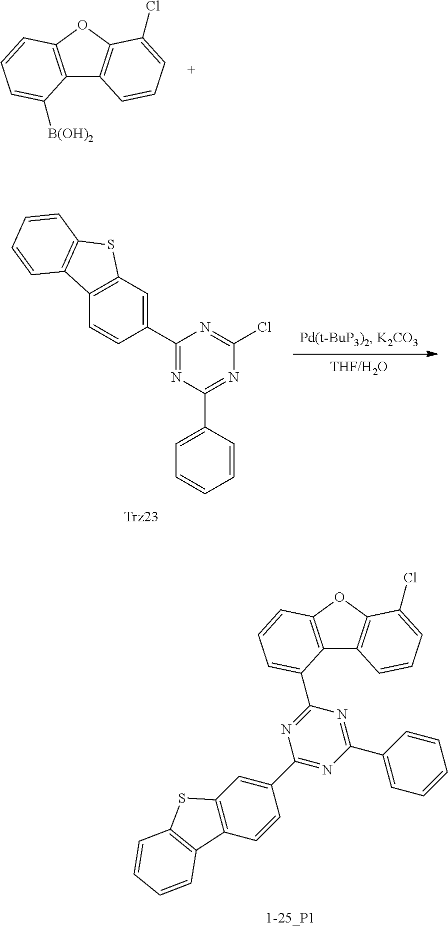 [SMILES: OB(O)c1cccc2oc3c(Cl)cccc3c12 ; Clc1nc(-c2ccccc2)nc(-c2ccc3c(c2)sc2ccccc23)n1 ; "THF/H2O" ; Clc1cccc2c1oc1cccc(-c3nc(-c4ccccc4)nc(-c4ccc5c(c4)sc4ccccc45)n3)c12]

(6-Chlorodibenzo[b,d]furan-1-yl)boronic acid (15 g, 60.9 mmol) and Compound Trz23 (25.2 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred and then bis(tri-tert-butylphosphine)palladium(0) (0.3 g, 0.6 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 20.7 g of Compound 1-25_P1. (Yield: 63%, MS: [M+H]⁺=540).

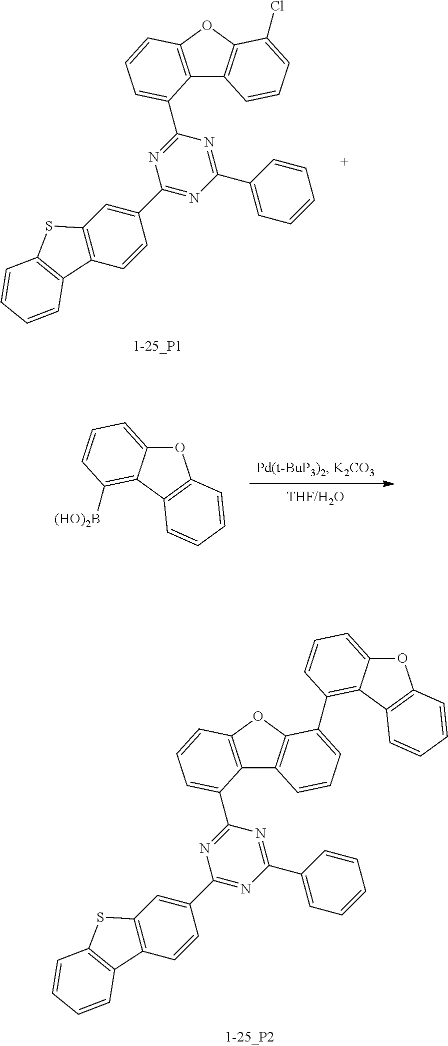 [SMILES: Clc1cccc2c1oc1cccc(-c3nc(-c4ccccc4)nc(-c4ccc5c(c4)sc4ccccc45)n3)c12 ; OB(O)c1cccc2oc3ccccc3c12 ; "THF/H2O" ; c1ccc(-c2nc(-c3ccc4c(c3)sc3ccccc34)nc(-c3cccc4oc5c(-c6cccc7oc8ccccc8c67)cccc5c34)n2)cc1]

Compound 1-25_P1 (15 g, 27.8 mmol) and dibenzo[b,d]furan-1-ylboronic acid (6.2 g, 29.2 mmol) were added to 300 ml of THF, and the mixture was stirred and refluxed. Then, potassium carbonate (11.5 g, 83.3 mmol) was dissolved in 35 ml of water and added thereto, and the mixture was sufficiently stirred and then bis(tri-tert-butylphosphine)palladium(0) (0.1 g, 0.3 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 11.4 g of Compound 1-25_P2. (Yield: 61%, MS: [M+H]⁺=672).

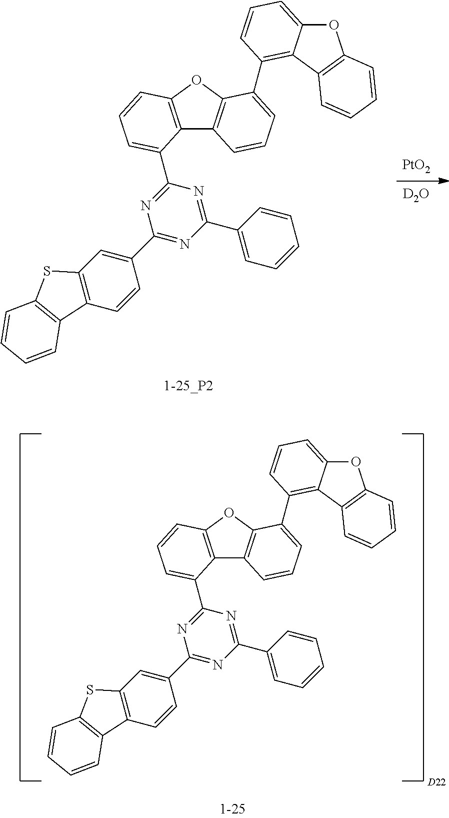

Compound 1-25_P2 (10 g, 14.9 mmol), PtO₂ (1 g, 4.5 mmol) and D₂O (74 ml) were added to a shaker tube, and then the tube was sealed and heated at 250° C. and 600 psi for 12 hours. When the reaction was completed, chloroform was added, and the reaction solution was transferred to a separatory funnel, and extracted. The extract was dried over MgSO₄ and concentrated, and then the sample was purified by silica gel column chromatography to prepare 3.7 g of Compound 1-25. (Yield: 36%, MS: [M+H]⁺=695)

### Synthesis Example 1-26

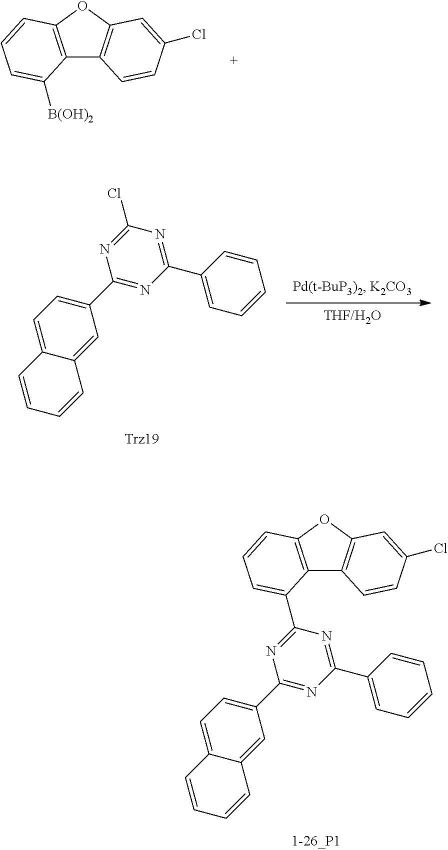 [SMILES: OB(O)c1cccc2oc3cc(Cl)ccc3c12 ; Clc1nc(-c2ccccc2)nc(-c2ccc3ccccc3c2)n1 ; "THF/H2O" ; Clc1ccc2c(c1)oc1cccc(-c3nc(-c4ccccc4)nc(-c4ccc5ccccc5c4)n3)c12]

(7-Chlorodibenzo[b,d]furan-1-yl)boronic acid (15 g, 60.9 mmol) and Compound Trz19 (20.3 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred and then bis(tri-tert-butylphosphine)palladium(0) (0.3 g, 0.6 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 17.6 g of Compound 1-26_P1. (Yield: 60%, MS: [M+H]⁺=484).

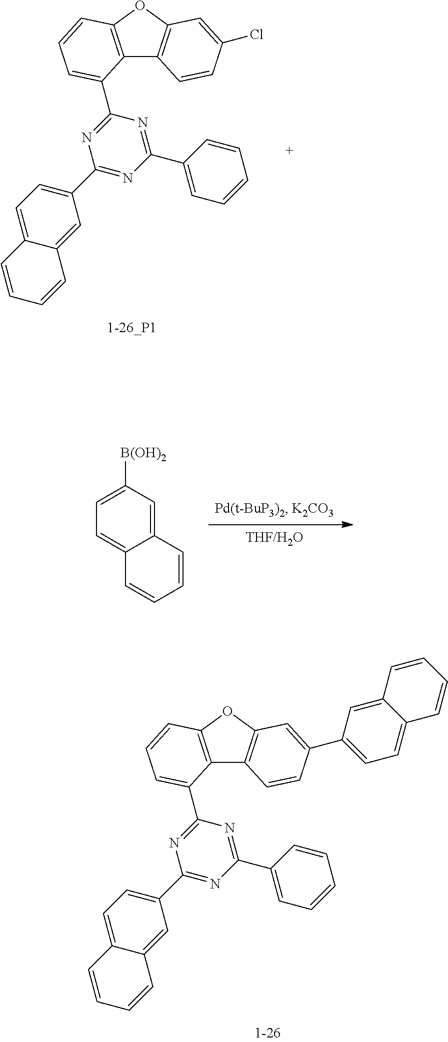 [SMILES: Clc1ccc2c(c1)oc1cccc(-c3nc(-c4ccccc4)nc(-c4ccc5ccccc5c4)n3)c12 ; OB(O)c1ccc2ccccc2c1 ; "THF/H2O" ; c1ccc(-c2nc(-c3ccc4ccccc4c3)nc(-c3cccc4oc5cc(-c6ccc7ccccc7c6)ccc5c34)n2)cc1]

Compound 1-26_P1 (15 g, 31 mmol) and naphthalen-2-ylboronic acid (5.6 g, 32.5 mmol) were added to 300 ml of THF, and the mixture was stirred and refluxed. Then, potassium carbonate (12.9 g, 93 mmol) was dissolved in 39 ml of water and added thereto, and the mixture was sufficiently stirred and then bis(tri-tert-butylphosphine)palladium(0) (0.2 g, 0.3 mmol) was added. After the reaction for 2 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 13 g of Compound 1-26. (Yield: 73%, MS: [M+H]⁺=576).

### Synthesis Example 1-27

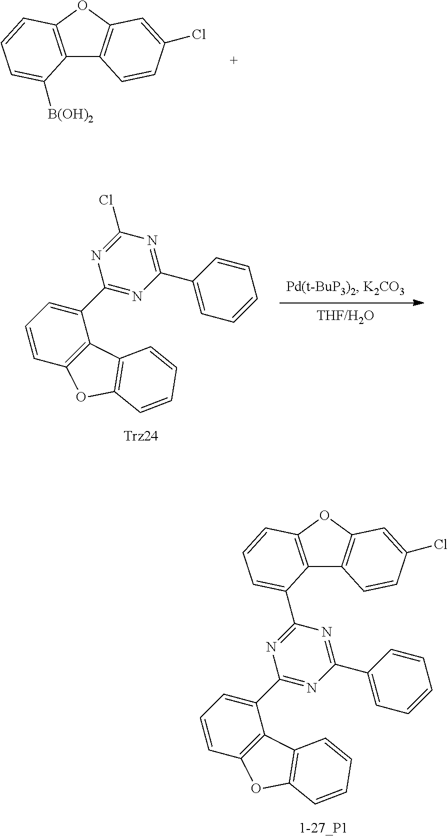 [SMILES: OB(O)c1cccc2oc3cc(Cl)ccc3c12 ; Clc1nc(-c2ccccc2)nc(-c2cccc3oc4ccccc4c23)n1 ; "THF/H2O" ; Clc1ccc2c(c1)oc1cccc(-c3nc(-c4ccccc4)nc(-c4cccc5oc6ccccc6c45)n3)c12]

(7-Chlorodibenzo[b,d]furan-1-yl)boronic acid (15 g, 60.9 mmol) and Compound Trz24 (22.9 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred and then bis(tri-tert-butylphosphine)palladium(0) (0.3 g, 0.6 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 20.4 g of Compound 1-27_P1. (Yield: 64%, MS: [M+H]⁺=524).

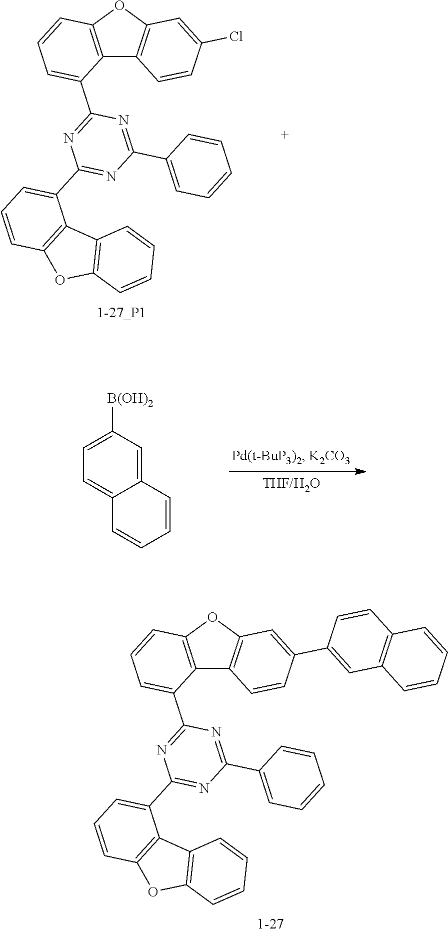 [SMILES: Clc1ccc2c(c1)oc1cccc(-c3nc(-c4ccccc4)nc(-c4cccc5oc6ccccc6c45)n3)c12 ; OB(O)c1ccc2ccccc2c1 ; c1ccc(-c2nc(-c3cccc4oc5ccccc5c34)nc(-c3cccc4oc5cc(-c6ccc7ccccc7c6)ccc5c34)n2)cc1]

Compound 1-27_P1 (15 g, 28.6 mmol) and naphthalen-2-ylboronic acid (5.2 g, 30.1 mmol) were added to 300 ml of THF, and the mixture was stirred and refluxed. Then, potassium carbonate (11.9 g, 85.9 mmol) was dissolved in 36 ml of water and added thereto, and the mixture was sufficiently stirred and then bis(tri-tert-butylphosphine)palladium(0) (0.1 g, 0.3 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 10.9 g of Compound 1-27. (Yield: 62%, MS: [M+H]⁺=616).

### Synthesis Example 1-28

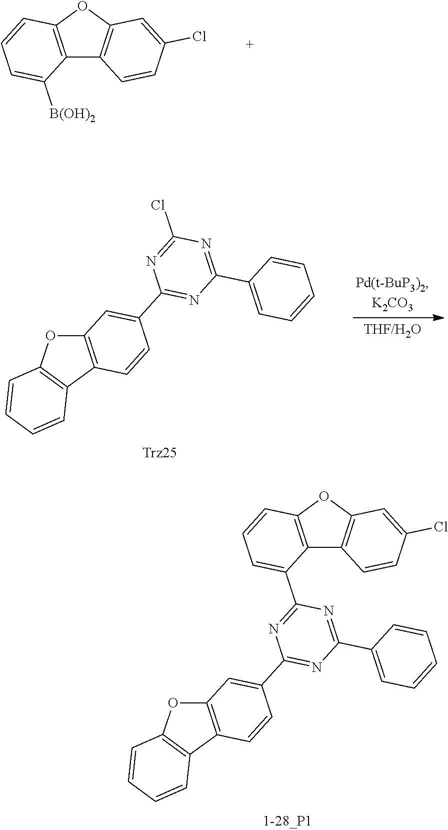 [SMILES: OB(O)c1cccc2oc3cc(Cl)ccc3c12 ; Clc1nc(-c2ccccc2)nc(-c2ccc3c(c2)oc2ccccc23)n1 ; Clc1ccc2c(c1)oc1cccc(-c3nc(-c4ccccc4)nc(-c4ccc5c(c4)oc4ccccc45)n3)c12]

(7-Chlorodibenzo[b,d]furan-1-yl)boronic acid (15 g, 60.9 mmol) and Compound Trz25 (22.9 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred and then bis(tri-tert-butylphosphine)palladium(0) (0.3 g, 0.6 mmol) was added. After the reaction for 2 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 22.6 g of Compound 1-28_P1. (Yield: 71%, MS: [M+H]⁺=524).

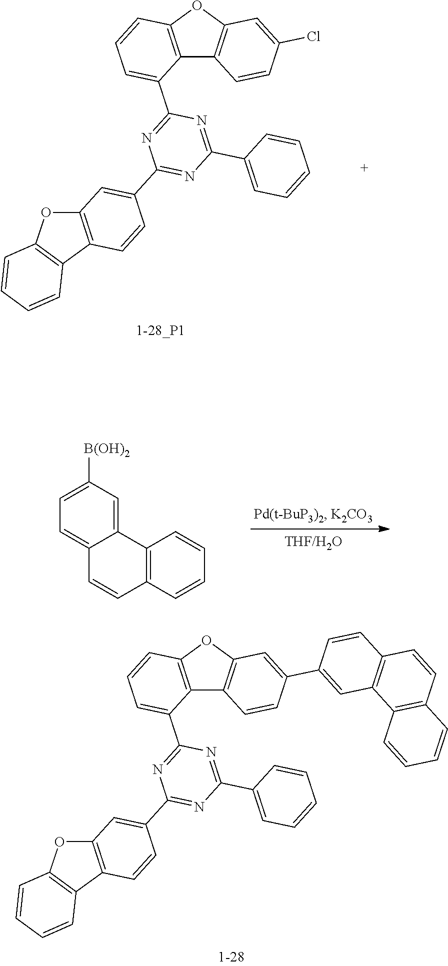 [SMILES: Clc1ccc2c(c1)oc1cccc(-c3nc(-c4ccccc4)nc(-c4ccc5c(c4)oc4ccccc45)n3)c12 ; OB(O)c1ccc2ccc3ccccc3c2c1 ; c1ccc(-c2nc(-c3ccc4c(c3)oc3ccccc34)nc(-c3cccc4oc5cc(-c6ccc7ccc8ccccc8c7c6)ccc5c34)n2)cc1]

Compound 1-28_P1 (15 g, 28.6 mmol) and phenanthren-3-ylboronic acid (6.7 g, 30.1 mmol) were added to 300 ml of THF, and the mixture was stirred and refluxed. Then, potassium carbonate (11.9 g, 85.9 mmol) was dissolved in 36 ml of water and added thereto, and the mixture was sufficiently stirred and then bis(tri-tert-butylphosphine)palladium(0) (0.1 g, 0.3 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 12.9 g of Compound 1-28. (Yield: 68%, MS: [M+H]⁺=666).

### Synthesis Example 1-29

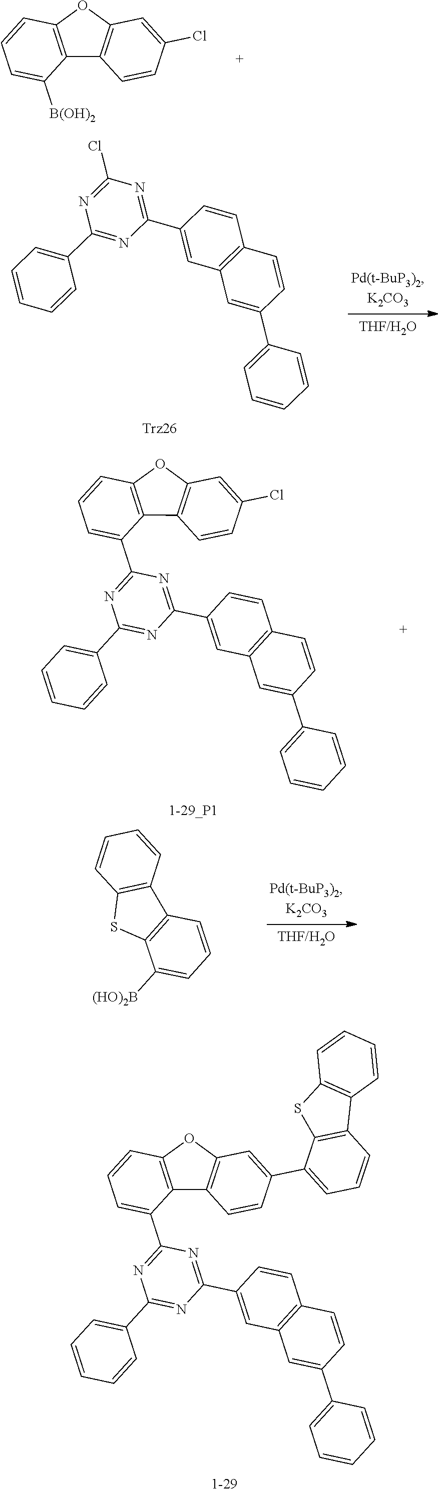 [SMILES: OB(O)c1cccc2oc3cc(Cl)ccc3c12 ; Clc1nc(-c2ccccc2)nc(-c2ccc3ccc(-c4ccccc4)cc3c2)n1 ; Clc1ccc2c(c1)oc1cccc(-c3nc(-c4ccccc4)nc(-c4ccc5ccc(-c6ccccc6)cc5c4)n3)c12 ; OB(O)c1cccc2c1sc1ccccc12 ; c1ccc(-c2ccc3ccc(-c4nc(-c5ccccc5)nc(-c5cccc6oc7cc(-c8cccc9c8sc8ccccc89)ccc7c56)n4)cc3c2)cc1]

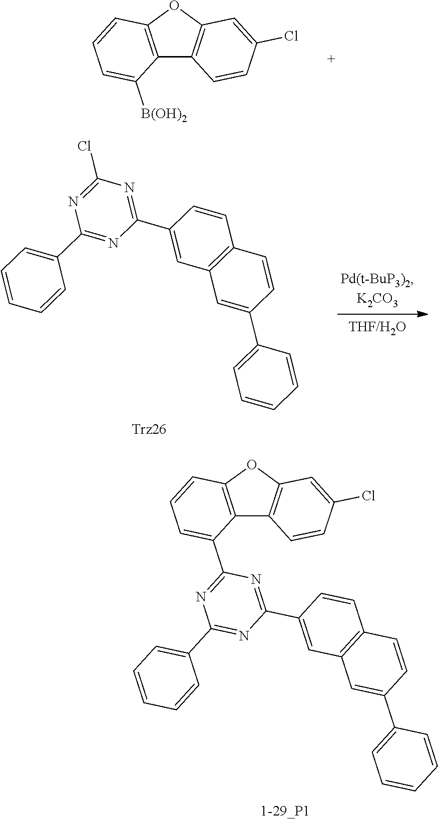 [SMILES: OB(O)c1cccc2oc3cc(Cl)ccc3c12 ; Clc1nc(-c2ccccc2)nc(-c2ccc3ccc(-c4ccccc4)cc3c2)n1 ; Clc1ccc2c(c1)oc1cccc(-c3nc(-c4ccccc4)nc(-c4ccc5ccc(-c6ccccc6)cc5c4)n3)c12]

(7-Chlorodibenzo[b,d]furan-1-yl)boronic acid (15 g, 60.9 mmol) and Compound Trz26 (25.2 g, 63.9 mmol) were added to 300 ml of THE, and the mixture was stirred and refluxed. Then, potassium carbonate (25.2 g, 182.6 mmol) was dissolved in 76 ml of water and added thereto, and the mixture was sufficiently stirred and then bis(tri-tert-butylphosphine)palladium(0) (0.3 g, 0.6 mmol) was added. After the reaction for 2 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 21.1 g of Compound 1-29_P1. (Yield: 62%, MS: [M+H]⁺=560).

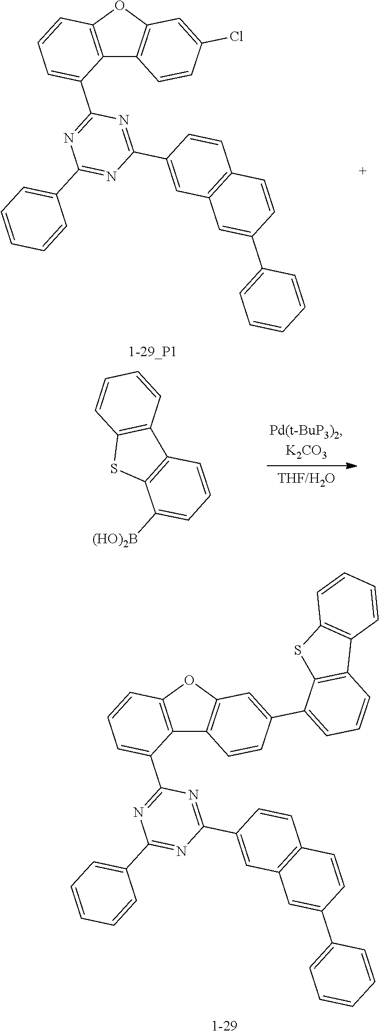 [SMILES: Clc1ccc2c(c1)oc1cccc(-c3nc(-c4ccccc4)nc(-c4ccc5ccc(-c6ccccc6)cc5c4)n3)c12 ; OB(O)c1cccc2c1sc1ccccc12 ; c1ccc(-c2ccc3ccc(-c4nc(-c5ccccc5)nc(-c5cccc6oc7cc(-c8cccc9c8sc8ccccc89)ccc7c56)n4)cc3c2)cc1]

Compound 1-29_P1 (15 g, 26.8 mmol) and dibenzo[b,d]thiophen-4-ylboronic acid (6.4 g, 28.1 mmol) were added to 300 ml of THF, and the mixture was stirred and refluxed. Then, potassium carbonate (11.1 g, 80.3 mmol) was dissolved in 33 ml of water and added thereto, and the mixture was sufficiently stirred and then bis(tri-tert-butylphosphine)palladium(0) (0.1 g, 0.3 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 12.3 g of Compound 1-29. (Yield: 65%, MS: [M+H]⁺=708).

### Synthesis Example 1-30

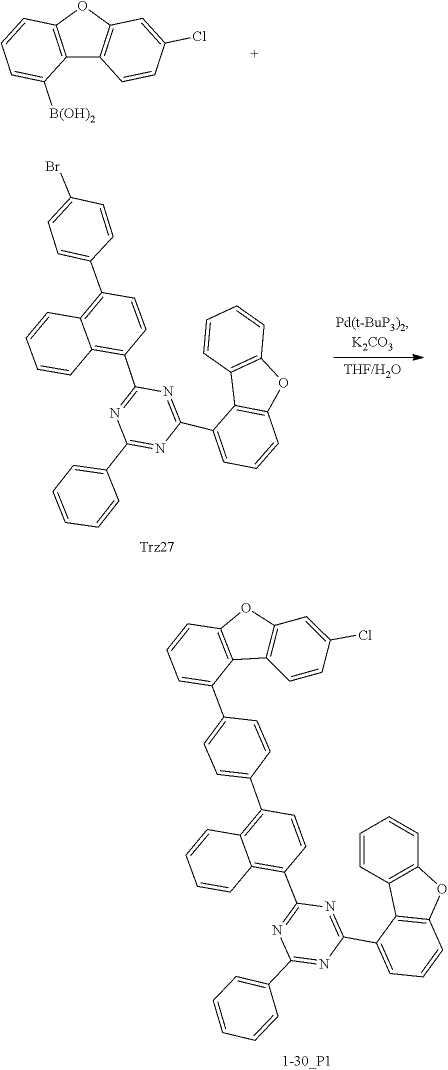 [SMILES: OB(O)c1cccc2oc3cc(Cl)ccc3c12 ; Brc1ccc(-c2ccc(-c3nc(-c4ccccc4)nc(-c4cccc5oc6ccccc6c45)n3)c3ccccc23)cc1 ; Clc1ccc2c(c1)oc1cccc(-c3ccc(-c4ccc(-c5nc(-c6ccccc6)nc(-c6cccc7oc8ccccc8c67)n5)c5ccccc45)cc3)c12]

(7-Chlorodibenzo[b,d]furan-1-yl)boronic acid (15 g, 60.9 mmol) and Compound Trz27 (38.6 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred and then bis(tri-tert-butylphosphine)palladium(0) (0.3 g, 0.6 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 29.1 g of Compound 1-30_P1. (Yield: 66%, MS: [M+H]⁺=726).

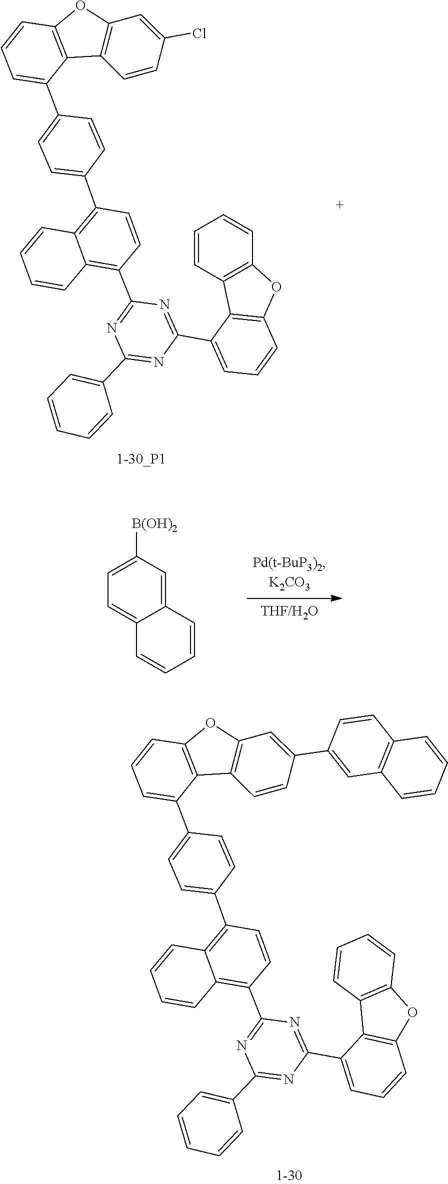 [SMILES: Clc1ccc2c(c1)oc1cccc(-c3ccc(-c4ccc(-c5nc(-c6ccccc6)nc(-c6cccc7oc8ccccc8c67)n5)c5ccccc45)cc3)c12 ; OB(O)c1ccc2ccccc2c1 ; c1ccc(-c2nc(-c3ccc(-c4ccc(-c5cccc6oc7cc(-c8ccc9ccccc9c8)ccc7c56)cc4)c4ccccc34)nc(-c3cccc4oc5ccccc5c34)n2)cc1]

Compound 1-30_P1 (15 g, 20.7 mmol) and naphthalen-2-ylboronic acid (3.7 g, 21.7 mmol) were added to 300 ml of THF, and the mixture was stirred and refluxed. Then, potassium carbonate (8.6 g, 62 mmol) was dissolved in 26 ml of water and added thereto, and the mixture was sufficiently stirred and then bis(tri-tert-butylphosphine)palladium(0) (0.1 g, 0.2 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 10.6 g of Compound 1-30. (Yield: 63%, MS: [M+H]⁺=818).

### Synthesis Example 1-31

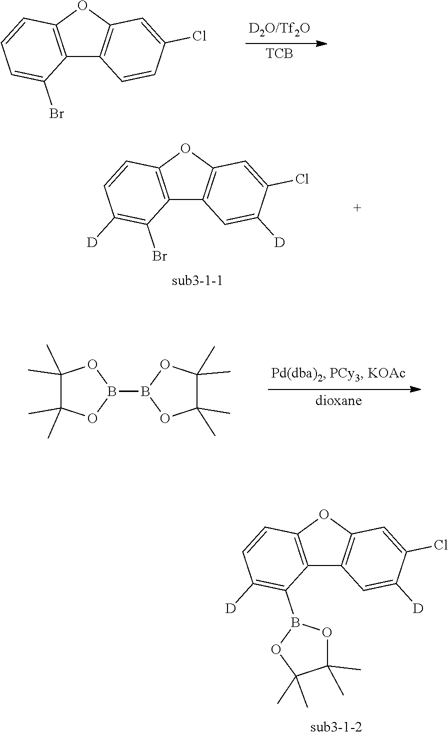

Trifluoromethanesulfonic anhydride (30.1 g, 106.6 mmol) and deuterium oxide (10.7 g, 532.8 mmol) were added at 0° C. and stirred for 5 hours to prepare a solution. 1-Bromo-7-chlorodibenzo[b,d]furan (15 g, 53.3 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of 1-bromo-7-chlorodibenzo[b,d]furan and 1,2,4-trichlorobenzene, and the mixture was stirred while heating up to 140° C. and then keeping that temperature. After the reaction for 3 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 6 g of compound Sub3-1-1. (Yield: 40%, MS: [M+H]⁺=283)

Compound Sub3-1-1 (15 g, 52.9 mmol) and bis(pinacolato)diboron (14.8 g, 58.2 mmol) were added to 300 ml of 1,4-dioxane, and the mixture was stirred under reflux. Then, potassium acetate (7.8 g, 79.4 mmol) was added thereto, the mixture was sufficiently stirred, and then bis(dibenzylideneacetone) palladium(0) (0.9 g, 1.6 mmol) and tricyclohexylphosphine (0.9 g, 3.2 mmol) were added. After the reaction for 4 hours, the reaction mixture was cooled to room temperature, the organic layer was separated using chloroform and water, and then the organic layer was distilled. This was dissolved again in chloroform, washed twice with water, and the organic layer was then separated. Anhydrous magnesium sulfate was added thereto, stirred, then filtered, and the filtrate was distilled under reduced pressure. The concentrated compound was purified by silica gel column chromatography to prepare 11.4 g of Compound Sub3-1-2. (Yield: 65%, MS: [M+H]⁺=331)

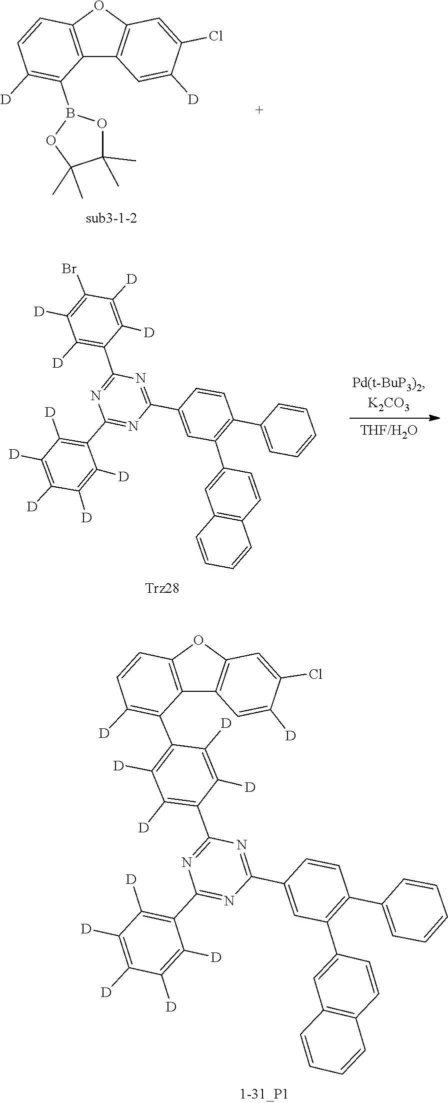 [SMILES: [2H]c1cc2c(cc1Cl)oc1ccc([2H])c(B3OC(C)(C)C(C)(C)O3)c12 ; [2H]c1c([2H])c([2H])c(-c2nc(-c3ccc(-c4ccccc4)c(-c4ccc5ccccc5c4)c3)nc(-c3c([2H])c([2H])c(Br)c([2H])c3[2H])n2)c([2H])c1[2H] ; [2H]c1cc2c(cc1Cl)oc1ccc([2H])c(-c3c([2H])c([2H])c(-c4nc(-c5ccc(-c6ccccc6)c(-c6ccc7ccccc7c6)c5)nc(-c5c([2H])c([2H])c([2H])c([2H])c5[2H])n4)c([2H])c3[2H])c12]

Compound Sub3-1-2 (15 g, 45.4 mmol) and Compound Trz28 (28.6 g, 47.6 mmol) were added to 300 ml of THF, and the mixture was stirred and refluxed. Then, potassium carbonate (18.8 g, 136.1 mmol) was dissolved in 56 ml of water and added thereto, and the mixture was sufficiently stirred and then bis(tri-tert-butylphosphine)palladium(0) (0.2 g, 0.5 mmol) was added. After the reaction for 2 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 20.3 g of Compound 1-31_P1. (Yield: 62%, MS: [M+H]⁺=723).

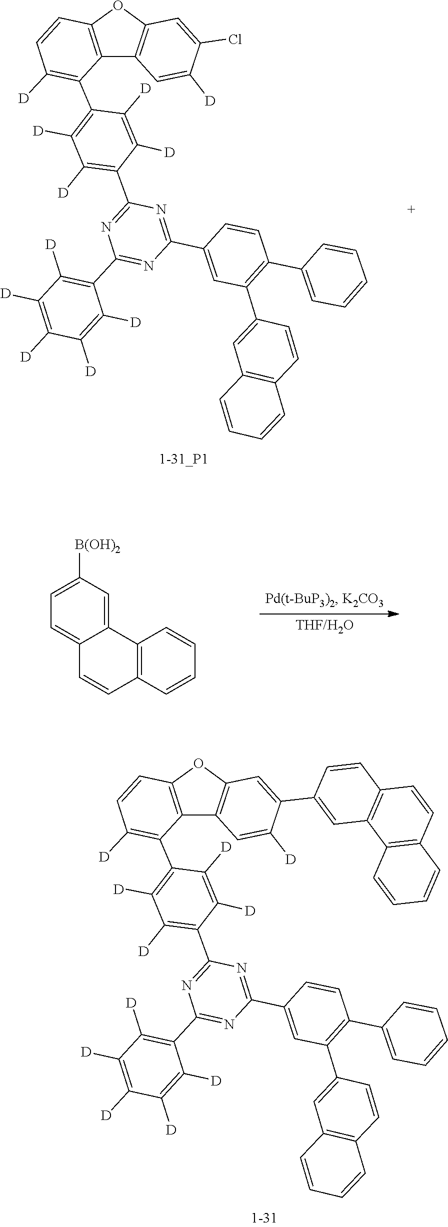 [SMILES: [2H]c1cc2c(cc1Cl)oc1ccc([2H])c(-c3c([2H])c([2H])c(-c4nc(-c5ccc(-c6ccccc6)c(-c6ccc7ccccc7c6)c5)nc(-c5c([2H])c([2H])c([2H])c([2H])c5[2H])n4)c([2H])c3[2H])c12 ; OB(O)c1ccc2ccc3ccccc3c2c1 ; [2H]c1cc2c(cc1-c1ccc3ccc4ccccc4c3c1)oc1ccc([2H])c(-c3c([2H])c([2H])c(-c4nc(-c5ccc(-c6ccccc6)c(-c6ccc7ccccc7c6)c5)nc(-c5c([2H])c([2H])c([2H])c([2H])c5[2H])n4)c([2H])c3[2H])c12]

Compound 1-31_P1 (15 g, 20.7 mmol) and phenanthren-3-ylboronic acid (4.8 g, 21.8 mmol) were added to 300 ml of THF, and the mixture was stirred and refluxed. Then, potassium carbonate (8.6 g, 62.2 mmol) was dissolved in 26 ml of water and added thereto, and the mixture was sufficiently stirred and then bis(tri-tert-butylphosphine)palladium(0) (0.1 g, 0.2 mmol) was added. After the reaction for 2 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 11.5 g of Compound 1-31. (Yield: 64%, MS: [M+H]⁺=866).

### Synthesis Example 1-32

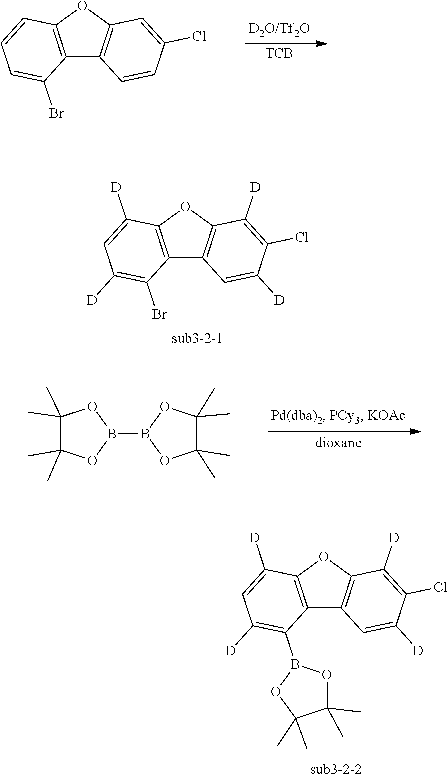

Trifluoromethanesulfonic anhydride (60.1 g, 213.1 mmol) and deuterium oxide (21.4 g, 1065.6 mmol) were added at 0° C. and stirred for hours to prepare a solution. 1-Bromo-7-chlorodibenzo[b,d]furan (15 g, 53.3 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of 1-bromo-7-chlorodibenzo[b,d]furan and 1,2,4-trichlorobenzene, and the mixture was stirred while heating up to 140° C. and then keeping that temperature. After the reaction for 10 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 6.7 g of Compound Sub3-2-1. (Yield: 44%, MS: [M+H]⁺=285)

Compound Sub3-2-1 (15 g, 52.5 mmol) and bis(pinacolato)diboron (14.7 g, 57.8 mmol) were added to 300 ml of 1,4-dioxane, and the mixture was stirred under reflux. Then, potassium acetate (7.7 g, 78.8 mmol) was added thereto, the mixture was sufficiently stirred, and then bis(dibenzylideneacetone) palladium(0) (0.9 g, 1.6 mmol) and tricyclohexylphosphine (0.9 g, 3.2 mmol) were added. After the reaction for 6 hours, the reaction mixture was cooled to room temperature, the organic layer was separated using chloroform and water, and then the organic layer was distilled. This was dissolved again in chloroform, washed twice with water, and the organic layer was then separated. Anhydrous magnesium sulfate was added thereto, stirred, then filtered, and the filtrate was distilled under reduced pressure. The concentrated compound was purified by silica gel column chromatography to prepare 11.7 g of Compound Sub3-2-2. (Yield: 67%, MS: [M+H]⁺=333)

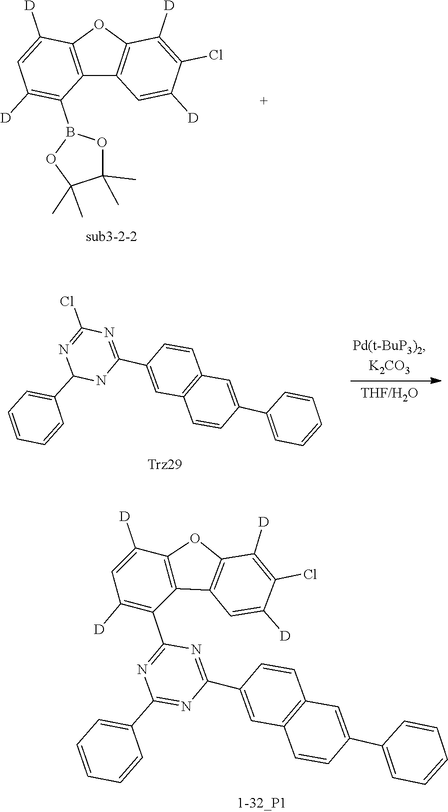

Compound Sub3-2-2 (15 g, 45.1 mmol) and Compound Trz29 (18.7 g, 47.4 mmol) were added to 300 ml of THF, and the mixture was stirred and refluxed. Then, potassium carbonate (18.7 g, 135.3 mmol) was dissolved in 56 ml of water and added thereto, and the mixture was sufficiently stirred and then bis(tri-tert-butylphosphine)palladium(0) (0.2 g, 0.5 mmol) was added. After the reaction for 2 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 18 g of Compound 1-32_P1. (Yield: 71%, MS: [M+H]⁺=564).

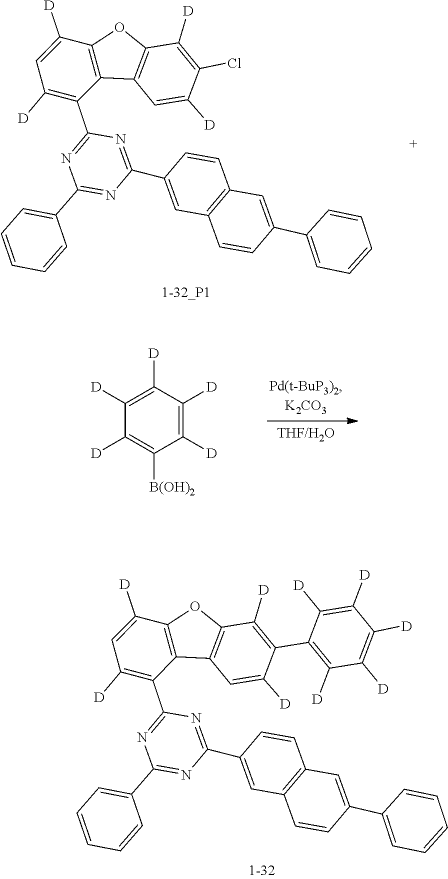 [SMILES: [2H]c1cc2c(oc3c([2H])cc([2H])c(-c4nc(-c5ccccc5)nc(-c5ccc6cc(-c7ccccc7)ccc6c5)n4)c32)c([2H])c1Cl ; [2H]c1c([2H])c([2H])c(B(O)O)c([2H])c1[2H] ; [2H]c1cc2c(oc3c([2H])cc([2H])c(-c4nc(-c5ccccc5)nc(-c5ccc6cc(-c7ccccc7)ccc6c5)n4)c32)c([2H])c1-c1c([2H])c([2H])c([2H])c([2H])c1[2H]]

Compound 1-32_P1 (15 g, 26.6 mmol) and (phenyl-d5)boronic acid (3.5 g, 27.9 mmol) were added to 300 ml of THF, and the mixture was stirred and refluxed. Then, potassium carbonate (11 g, 79.8 mmol) was dissolved in 33 ml of water and added thereto, and the mixture was sufficiently stirred and then bis(tri-tert-butylphosphine)palladium(0) (0.1 g, 0.3 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 11.4 g of Compound 1-32. (Yield: 70%, MS: [M+H]⁺=611).

### Synthesis Example 1-33

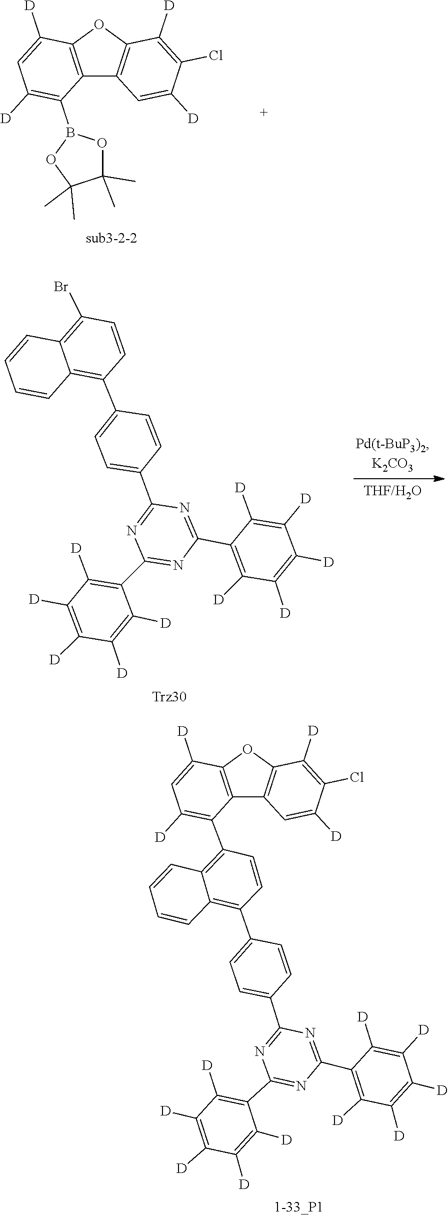 [SMILES: [2H]c1cc2c(oc3c([2H])cc([2H])c(B4OC(C)(C)C(C)(C)O4)c32)c([2H])c1Cl ; [2H]c1c([2H])c([2H])c(-c2nc(-c3ccc(-c4ccc(Br)c5ccccc45)cc3)nc(-c3c([2H])c([2H])c([2H])c([2H])c3[2H])n2)c([2H])c1[2H] ; [2H]c1cc2c(oc3c([2H])cc([2H])c(-c4ccc(-c5ccc(-c6nc(-c7c([2H])c([2H])c([2H])c([2H])c7[2H])nc(-c7c([2H])c([2H])c([2H])c([2H])c7[2H])n6)cc5)c5ccccc45)c32)c([2H])c1Cl]

Compound Sub3-2-2 (15 g, 45.1 mmol) and Compound Trz30 (24.8 g, 47.4 mmol) were added to 300 ml of THF, and the mixture was stirred and refluxed. Then, potassium carbonate (18.7 g, 135.3 mmol) was dissolved in 56 ml of water and added thereto, and the mixture was sufficiently stirred and then bis(tri-tert-butylphosphine)palladium(0) (0.2 g, 0.5 mmol) was added. After the reaction for 2 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 18.4 g of Compound 1-33_P1. (Yield: 63%, MS: [M+H]⁺=650).

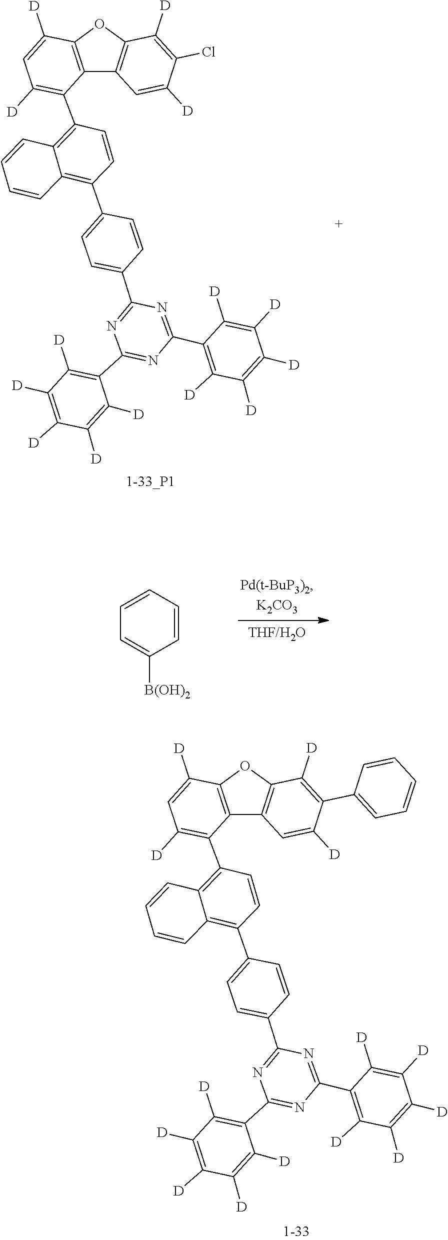 [SMILES: [2H]c1cc2c(oc3c([2H])cc([2H])c(-c4ccc(-c5ccc(-c6nc(-c7c([2H])c([2H])c([2H])c([2H])c7[2H])nc(-c7c([2H])c([2H])c([2H])c([2H])c7[2H])n6)cc5)c5ccccc45)c32)c([2H])c1Cl ; OB(O)c1ccccc1 ; [2H]c1cc2c(oc3c([2H])cc([2H])c(-c4ccc(-c5ccc(-c6nc(-c7c([2H])c([2H])c([2H])c([2H])c7[2H])nc(-c7c([2H])c([2H])c([2H])c([2H])c7[2H])n6)cc5)c5ccccc45)c32)c([2H])c1-c1ccccc1]

Compound 1-33_P1 (15 g, 23.1 mmol) and phenylboronic acid (3 g, 24.2 mmol) were added to 300 ml of THF, and the mixture was stirred and refluxed. Then, potassium carbonate (9.6 g, 69.2 mmol) was dissolved in 29 ml of water and added thereto, and the mixture was sufficiently stirred and then bis(tri-tert-butylphosphine)palladium(0) (0.1 g, 0.2 mmol) was added. After the reaction for 2 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 11.5 g of Compound 1-33. (Yield: 72%, MS: [M+H]⁺=692).

### Synthesis Example 1-34

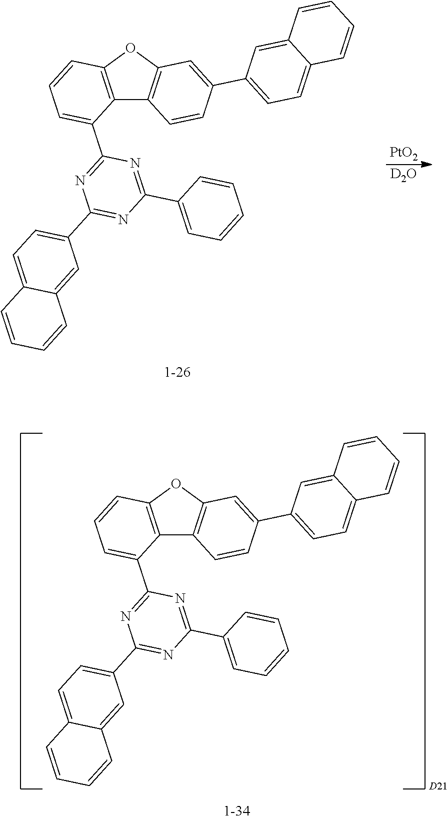

Compound 1-26 (10 g, 17.4 mmol), PtO₂ (1.2 g, 5.2 mmol) and D₂O (87 ml) were added to a shaker tube, and then the tube was sealed and heated at 250° C. and 600 psi for 12 hours. When the reaction was completed, chloroform was added, and the reaction solution was transferred to a separatory funnel, and extracted. The extract was dried over MgSO₄ and concentrated, and then the sample was purified by silica gel column chromatography to prepare 3.9 g of Compound 1-34. (Yield: 38%, MS: [M+H]⁺=598)

### Synthesis Example 1-35

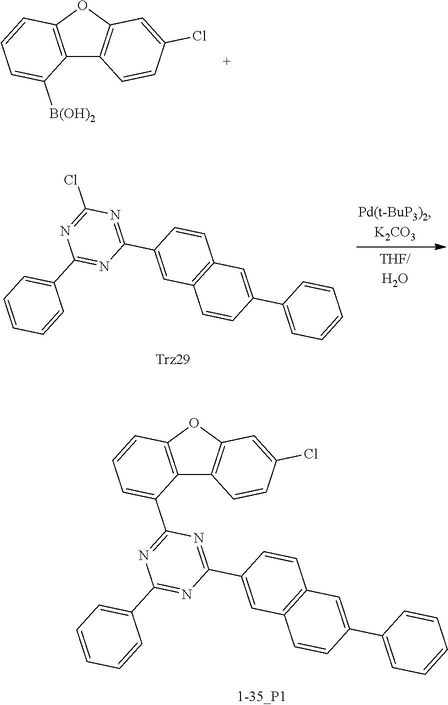 [SMILES: OB(O)c1cccc2oc3cc(Cl)ccc3c12 ; Clc1nc(-c2ccccc2)nc(-c2ccc3cc(-c4ccccc4)ccc3c2)n1 ; Clc1ccc2c(c1)oc1cccc(-c3nc(-c4ccccc4)nc(-c4ccc5cc(-c6ccccc6)ccc5c4)n3)c12]

(7-Chlorodibenzo[b,d]furan-1-yl)boronic acid (15 g, 60.9 mmol) and Compound Trz29 (25.2 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred and then bis(tri-tert-butylphosphine)palladium(0) (0.3 g, 0.6 mmol) was added. After the reaction for 2 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 23.8 g of Compound 1-35_P1. (Yield: 70%, MS: [M+H]⁺=560).

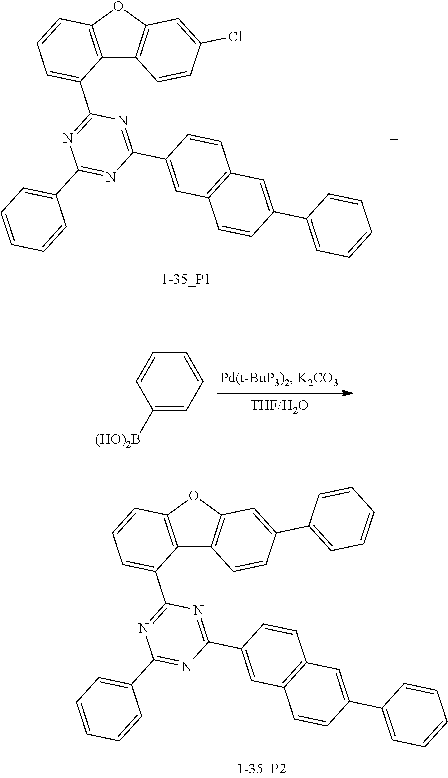 [SMILES: Clc1ccc2c(c1)oc1cccc(-c3nc(-c4ccccc4)nc(-c4ccc5cc(-c6ccccc6)ccc5c4)n3)c12 ; OB(O)c1ccccc1 ; c1ccc(-c2ccc3cc(-c4nc(-c5ccccc5)nc(-c5cccc6oc7cc(-c8ccccc8)ccc7c56)n4)ccc3c2)cc1]

Compound 1-35_P1 (15 g, 26.8 mmol) and phenylboronic acid (3.4 g, 28.1 mmol) were added to 300 ml of THF, and the mixture was stirred and refluxed. Then, potassium carbonate (11.1 g, 80.3 mmol) was dissolved in 33 ml of water and added thereto, and the mixture was sufficiently stirred and then bis(tri-tert-butylphosphine)palladium(0) (0.1 g, 0.3 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 11.3 g of Compound 1-35_P2. (Yield: 70%, MS: [M+H]⁺=602).

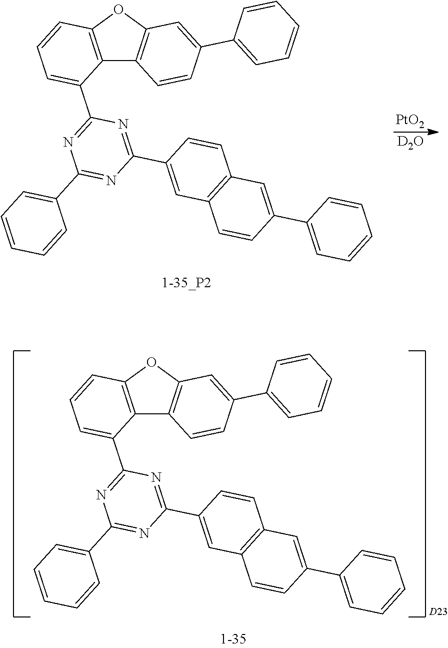

Compound 1-35_P2 (10 g, 16.6 mmol), PtO₂ (1.1 g, 5 mmol) and D₂O (83 ml) were added to a shaker tube, and then the tube was sealed and heated at 250° C. and 600 psi for 12 hours. When the reaction was completed, chloroform was added, and the reaction solution was transferred to a separatory funnel, and extracted. The extract was dried over MgSO₄ and concentrated, and then the sample was purified by silica gel column chromatography to prepare 3.7 g of Compound 1-35. (Yield: 36%, MS: [M+H]⁺=626)

### Synthesis Example 1-36

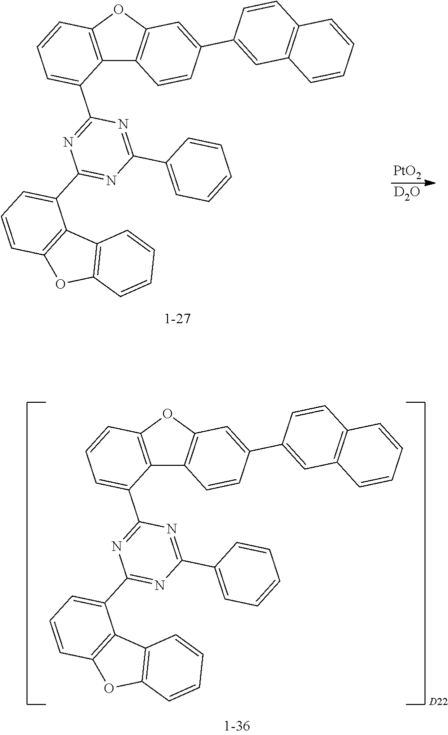

Compound 1-27 (10 g, 16.2 mmol), PtO₂ (1.1 g, 4.9 mmol) and D₂O (81 ml) were added to a shaker tube, and then the tube was sealed and heated at 250° C. and 600 psi for 12 hours. When the reaction was completed, chloroform was added, and the reaction solution was transferred to a separatory funnel, and extracted. The extract was dried over MgSO₄ and concentrated, and then the sample was purified by silica gel column chromatography to prepare 3.9 g of Compound 1-36. (Yield: 38%, MS: [M+H]⁺=639)

### Synthesis Example 1-37

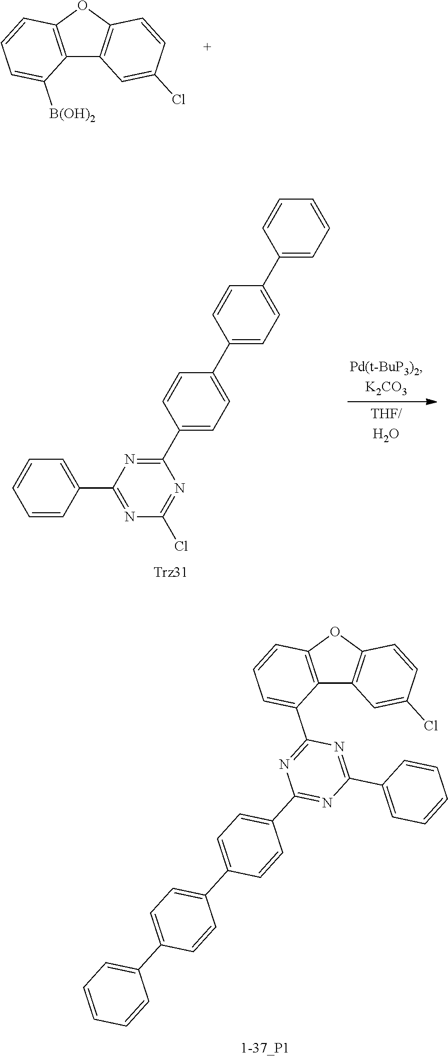 [SMILES: OB(O)c1cccc2oc3ccc(Cl)cc3c12 ; Clc1nc(-c2ccccc2)nc(-c2ccc(-c3ccc(-c4ccccc4)cc3)cc2)n1 ; Clc1ccc2oc3cccc(-c4nc(-c5ccccc5)nc(-c5ccc(-c6ccc(-c7ccccc7)cc6)cc5)n4)c3c2c1]

(8-Chlorodibenzo[b,d]furan-1-yl)boronic acid (15 g, 60.9 mmol) and Compound Trz31 (26.8 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred and then bis(tri-tert-butylphosphine)palladium(0) (0.3 g, 0.6 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 26.7 g of Compound 1-37_P1. (Yield: 75%, MS: [M+H]⁺=586).

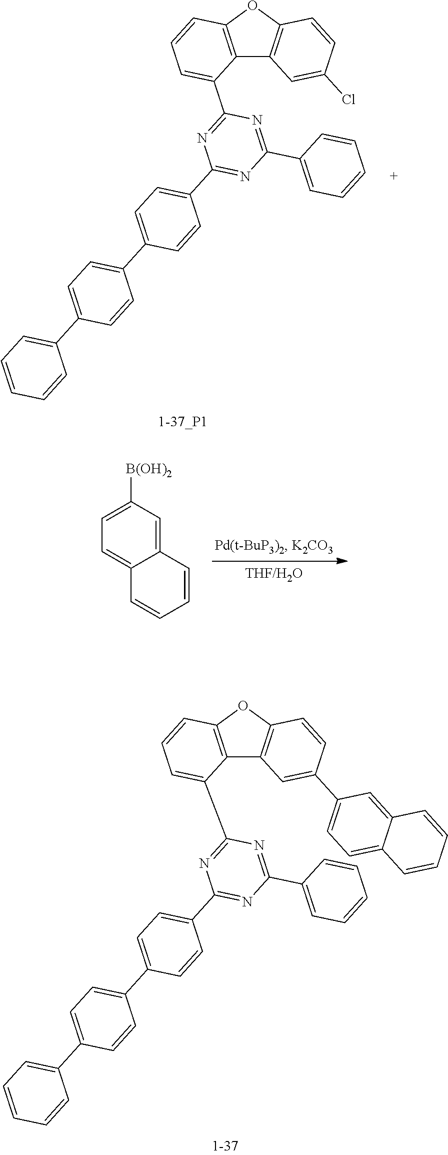 [SMILES: Clc1ccc2oc3cccc(-c4nc(-c5ccccc5)nc(-c5ccc(-c6ccc(-c7ccccc7)cc6)cc5)n4)c3c2c1 ; OB(O)c1ccc2ccccc2c1 ; c1ccc(-c2ccc(-c3ccc(-c4nc(-c5ccccc5)nc(-c5cccc6oc7ccc(-c8ccc9ccccc9c8)cc7c56)n4)cc3)cc2)cc1]

Compound 1-37_P1 (15 g, 25.6 mmol) and naphthalen-2-ylboronic acid (4.6 g, 26.9 mmol) were added to 300 ml of THF, and the mixture was stirred and refluxed. Then, potassium carbonate (10.6 g, 76.8 mmol) was dissolved in 32 ml of water and added thereto, and the mixture was sufficiently stirred and then bis(tri-tert-butylphosphine)palladium(0) (0.1 g, 0.3 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 12.7 g of Compound 1-37. (Yield: 73%, MS: [M+H]⁺=678).

### Synthesis Example 1-38

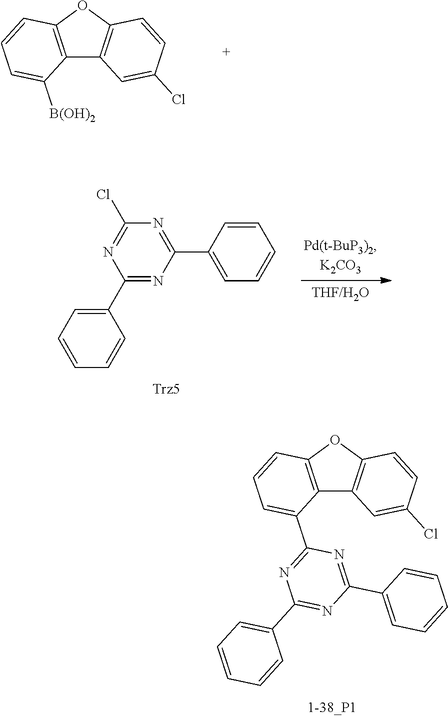 [SMILES: OB(O)c1cccc2oc3ccc(Cl)cc3c12 ; Clc1nc(-c2ccccc2)nc(-c2ccccc2)n1 ; Clc1ccc2oc3cccc(-c4nc(-c5ccccc5)nc(-c5ccccc5)n4)c3c2c1]

(8-Chlorodibenzo[b,d]furan-1-yl)boronic acid (15 g, 60.9 mmol) and Compound Trz5 (17.1 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred and then bis(tri-tert-butylphosphine)palladium(0) (0.3 g, 0.6 mmol) was added. After the reaction for 2 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 16.3 g of Compound 1-38_P1. (Yield: 62%, MS: [M+H]⁺=434).

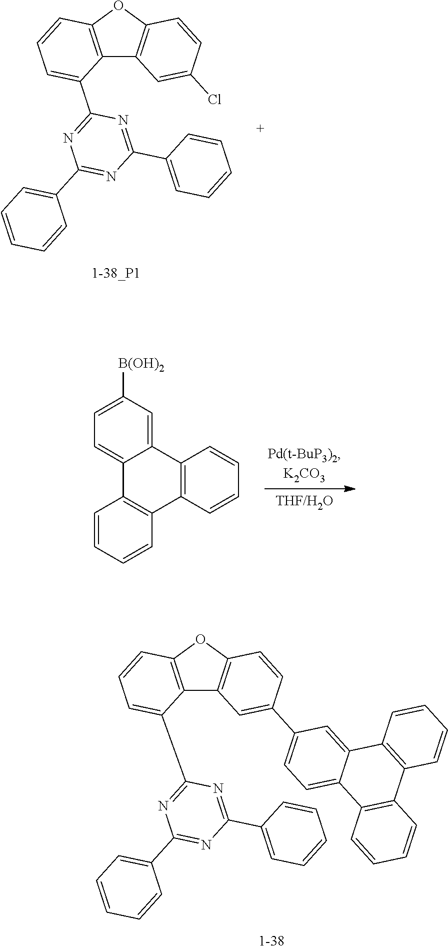 [SMILES: Clc1ccc2oc3cccc(-c4nc(-c5ccccc5)nc(-c5ccccc5)n4)c3c2c1 ; OB(O)c1ccc2c3ccccc3c3ccccc3c2c1 ; c1ccc(-c2nc(-c3ccccc3)nc(-c3cccc4oc5ccc(-c6ccc7c8ccccc8c8ccccc8c7c6)cc5c34)n2)cc1]

Compound 1-38_P1 (15 g, 34.6 mmol) and triphenylen-2-ylboronic acid (9.9 g, 36.3 mmol) were added to 300 ml of THF, and the mixture was stirred and refluxed. Then, potassium carbonate (14.3 g, 103.7 mmol) was dissolved in 43 ml of water and added thereto, and the mixture was sufficiently stirred and then bis(tri-tert-butylphosphine)palladium(0) (0.2 g, 0.3 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 14.7 g of Compound 1-38. (Yield: 68%, MS: [M+H]⁺=626).

### Synthesis Example 1-39

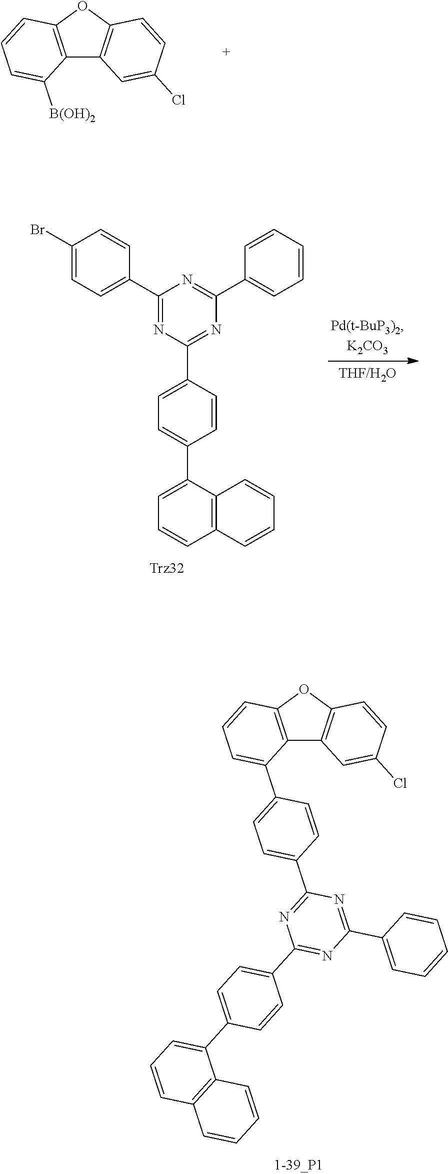 [SMILES: OB(O)c1cccc2oc3ccc(Cl)cc3c12 ; Brc1ccc(-c2nc(-c3ccccc3)nc(-c3ccc(-c4cccc5ccccc45)cc3)n2)cc1 ; Clc1ccc2oc3cccc(-c4ccc(-c5nc(-c6ccccc6)nc(-c6ccc(-c7cccc8ccccc78)cc6)n5)cc4)c3c2c1]

(8-Chlorodibenzo[b,d]furan-1-yl)boronic acid (15 g, 60.9 mmol) and Compound Trz32 (32.9 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred and then bis(tri-tert-butylphosphine)palladium(0) (0.3 g, 0.6 mmol) was added. After the reaction for 2 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 27.8 g of Compound 1-39_P1. (Yield: 72%, MS: [M+H]⁺=636).

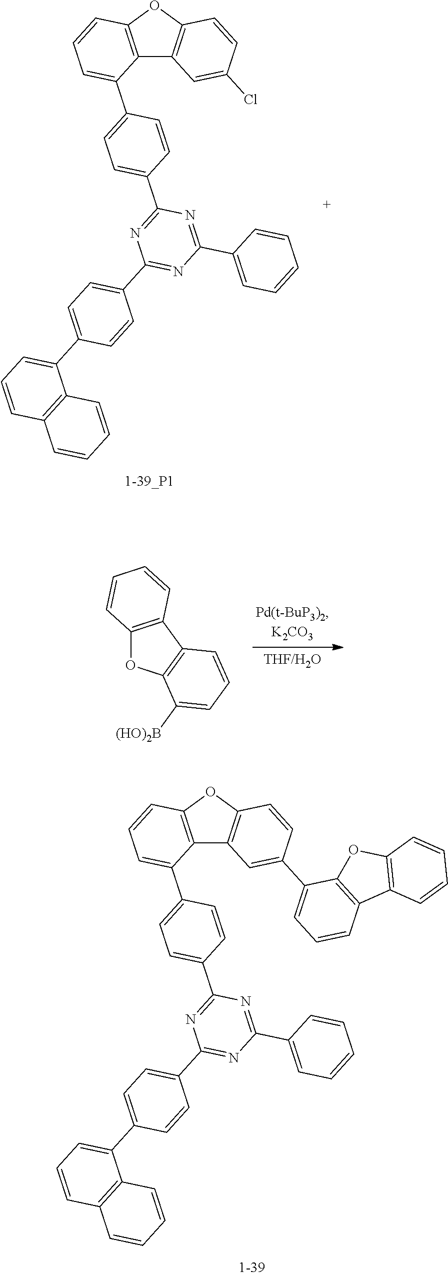 [SMILES: Clc1ccc2oc3cccc(-c4ccc(-c5nc(-c6ccccc6)nc(-c6ccc(-c7cccc8ccccc78)cc6)n5)cc4)c3c2c1 ; OB(O)c1cccc2c1oc1ccccc12 ; c1ccc(-c2nc(-c3ccc(-c4cccc5ccccc45)cc3)nc(-c3ccc(-c4cccc5oc6ccc(-c7cccc8c7oc7ccccc78)cc6c45)cc3)n2)cc1]

Compound 1-39_P1 (15 g, 23.6 mmol) and dibenzo[b,d]furan-4-ylboronic acid (5.2 g, 24.8 mmol) were added to 300 ml of THF, and the mixture was stirred and refluxed. Then, potassium carbonate (9.8 g, 70.7 mmol) was dissolved in 29 ml of water and added thereto, and the mixture was sufficiently stirred and then bis(tri-tert-butylphosphine)palladium(0) (0.1 g, 0.2 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 11.6 g of Compound 1-39. (Yield: 64%, MS: [M+H]⁺=769).

### Synthesis Example 1-40

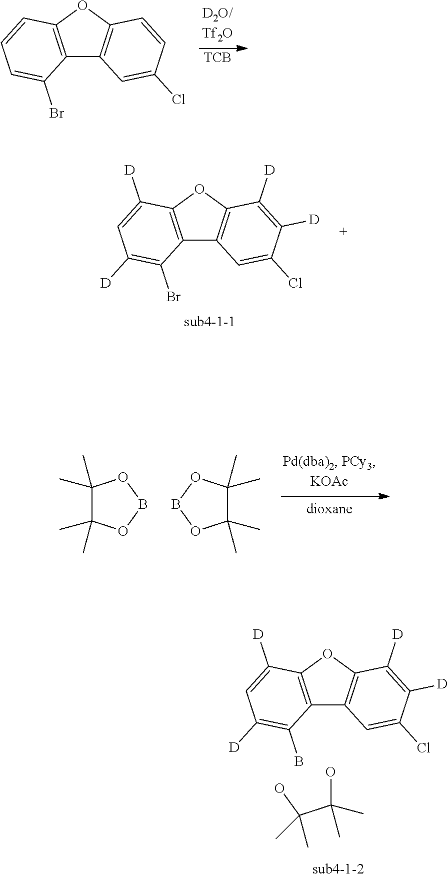

Trifluoromethanesulfonic anhydride (60.1 g, 213.1 mmol) and deuterium oxide (21.4 g, 1065.6 mmol) were added at 0° C. and stirred for hours to prepare a solution. 1-Bromo-8-chlorodibenzo[b,d]furan (15 g, 53.3 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of 1-bromo-8-chlorodibenzo[b,d]furan and 1,2,4-trichlorobenzene, and the mixture was stirred while heating up to 140° C. and then keeping that temperature. After the reaction for 10 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 6.4 g of Compound Sub4-1-1. (Yield: 42%, MS: [M+H]⁺=285)

Compound Sub4-1-1 (15 g, 52.5 mmol) and bis(pinacolato)diboron (14.7 g, 57.8 mmol) were added to 300 ml of 1,4-dioxane, and the mixture was stirred under reflux. Then, potassium acetate (7.7 g, 78.8 mmol) was added thereto, the mixture was sufficiently stirred, and then bis(dibenzylideneacetone) palladium(0) (0.9 g, 1.6 mmol) and tricyclohexylphosphine (0.9 g, 3.2 mmol) were added. After the reaction for 6 hours, the reaction mixture was cooled to room temperature, the organic layer was separated using chloroform and water, and then the organic layer was distilled. This was dissolved again in chloroform, washed twice with water, and the organic layer was then separated.

Anhydrous magnesium sulfate was added thereto, stirred, then filtered, and the filtrate was distilled under reduced pressure. The concentrated compound was purified by silica gel column chromatography to prepare 12 g of Compound Sub4-1-2. (Yield: 69%, MS: [M+H]⁺=333)

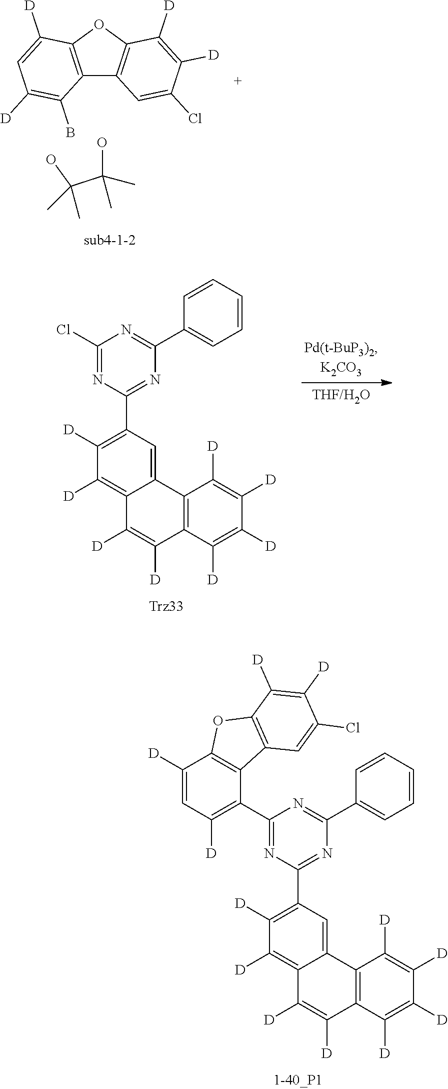

Compound Sub4-1-2 (15 g, 45.1 mmol) and Compound Trz33 (17.8 g, 47.4 mmol) were added to 300 ml of THE, and the mixture was stirred and refluxed. Then, potassium carbonate (18.7 g, 135.3 mmol) was dissolved in 56 ml of water and added thereto, and the mixture was sufficiently stirred and then bis(tri-tert-butylphosphine)palladium(0) (0.2 g, 0.5 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 16 g of Compound 1-40_P1. (Yield: 65%, MS: [M+H]⁺=546).

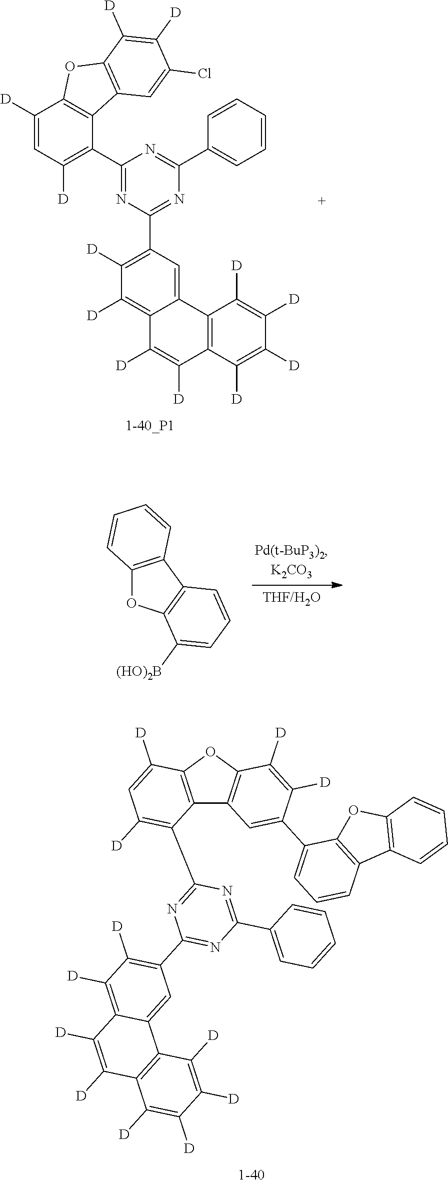 [SMILES: [2H]c1cc([2H])c2oc3c([2H])c([2H])c(Cl)cc3c2c1-c1nc(-c2ccccc2)nc(-c2cc3c(c([2H])c2[2H])c([2H])c([2H])c2c([2H])c([2H])c([2H])c([2H])c23)n1 ; OB(O)c1cccc2c1oc1ccccc12 ; [2H]c1cc([2H])c2oc3c([2H])c([2H])c(-c4cccc5c4oc4ccccc45)cc3c2c1-c1nc(-c2ccccc2)nc(-c2cc3c(c([2H])c2[2H])c([2H])c([2H])c2c([2H])c([2H])c([2H])c([2H])c23)n1]

Compound 1-40_P1 (15 g, 27.5 mmol) and dibenzo[b,d]furan-4-ylboronic acid (6.1 g, 28.8 mmol) were added to 300 ml of THF, and the mixture was stirred and refluxed. Then, potassium carbonate (11.4 g, 82.4 mmol) was dissolved in 34 ml of water and added thereto, and the mixture was sufficiently stirred and then bis(tri-tert-butylphosphine)palladium(0) (0.1 g, 0.3 mmol) was added. After the reaction for 2 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 11.5 g of Compound 1-40. (Yield: 62%, MS: [M+H]⁺=678).

### Synthesis Example 1-41

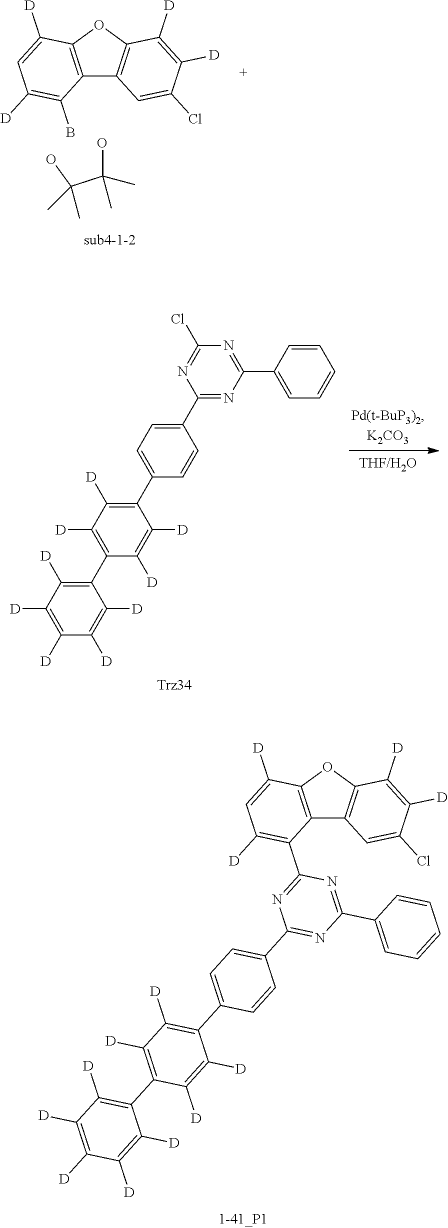

Compound Sub4-1-2 (15 g, 45.1 mmol) and Compound Trz34 (20.3 g, 47.4 mmol) were added to 300 ml of THF, and the mixture was stirred and refluxed. Then, potassium carbonate (18.7 g, 135.3 mmol) was dissolved in 56 ml of water and added thereto, and the mixture was sufficiently stirred and then bis(tri-tert-butylphosphine)palladium(0) (0.2 g, 0.5 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 19.4 g of Compound 1-41_P1. (Yield: 72%, MS: [M+H]⁺=599).

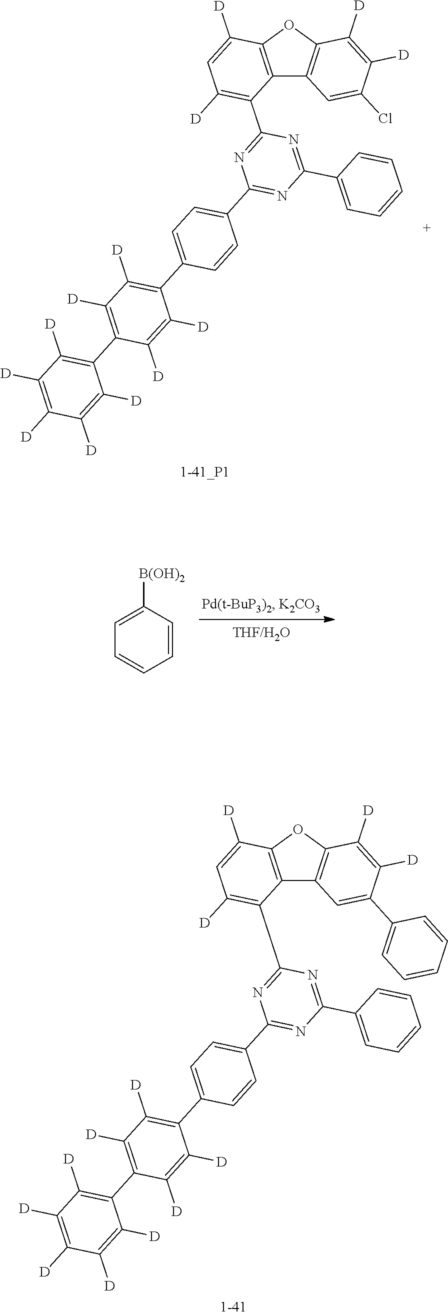 [SMILES: [2H]c1cc([2H])c2oc3c([2H])c([2H])c(Cl)cc3c2c1-c1nc(-c2ccccc2)nc(-c2ccc(-c3c([2H])c([2H])c(-c4c([2H])c([2H])c([2H])c([2H])c4[2H])c([2H])c3[2H])cc2)n1 ; OB(O)c1ccccc1 ; [2H]c1cc([2H])c2oc3c([2H])c([2H])c(-c4ccccc4)cc3c2c1-c1nc(-c2ccccc2)nc(-c2ccc(-c3c([2H])c([2H])c(-c4c([2H])c([2H])c([2H])c([2H])c4[2H])c([2H])c3[2H])cc2)n1]

Compound 1-41_P1 (15 g, 25 mmol) and phenylboronic acid (3.2 g, 26.3 mmol) were added to 300 ml of THF, and the mixture was stirred and refluxed. Then, potassium carbonate (10.4 g, 75.1 mmol) was dissolved in 31 ml of water and added thereto, and the mixture was sufficiently stirred and then bis(tri-tert-butylphosphine)palladium(0) (0.1 g, 0.3 mmol) was added. After the reaction for 2 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 9.8 g of Compound 1-41. (Yield: 61%, MS: [M+H]⁺=641).

### Synthesis Example 1-42

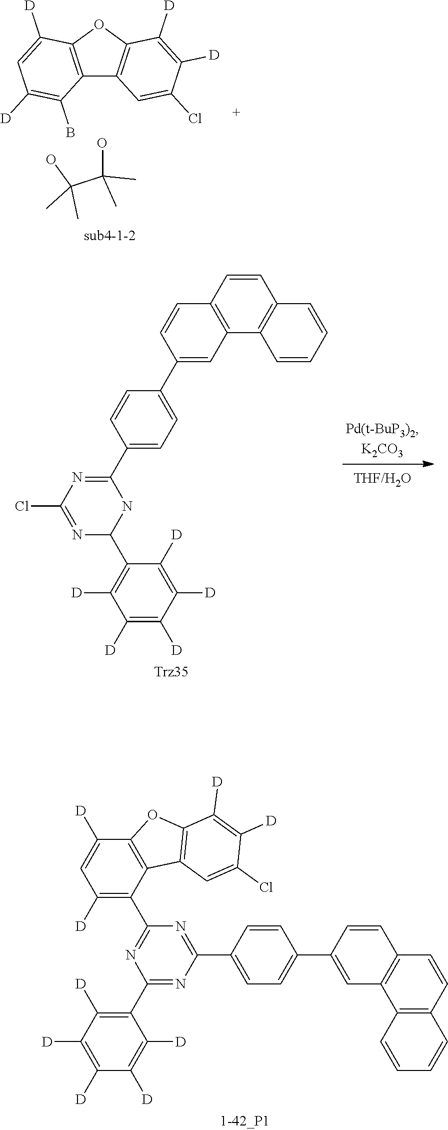

Compound Sub4-1-2 (15 g, 45.1 mmol) and Compound Trz35 (21.3 g, 47.4 mmol) were added to 300 ml of THF, and the mixture was stirred and refluxed. Then, potassium carbonate (18.7 g, 135.3 mmol) was dissolved in 56 ml of water and added thereto, and the mixture was sufficiently stirred and then bis(tri-tert-butylphosphine)palladium(0) (0.2 g, 0.5 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 17 g of Compound 1-42_P1. (Yield: 61%, MS: [M+H]⁺=619).

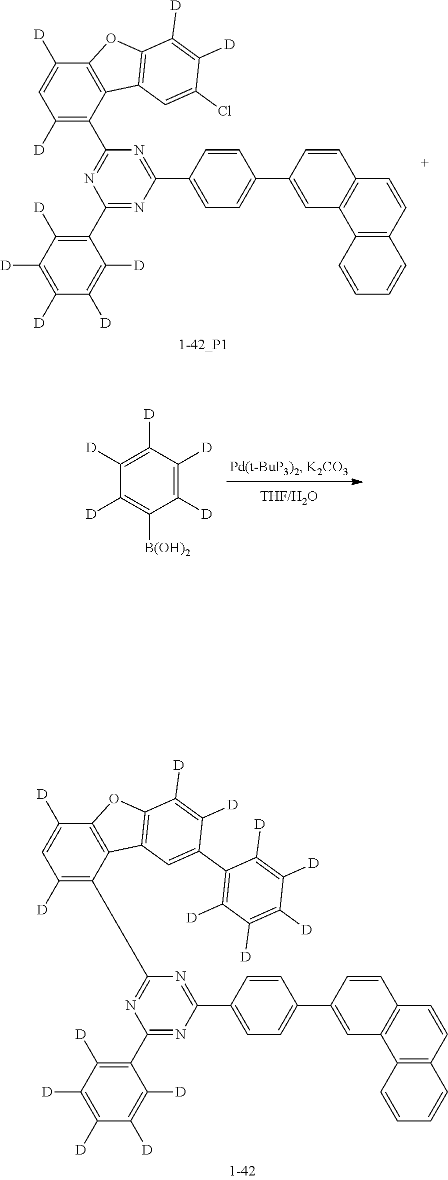

Compound 1-42_P1 (15 g, 24.2 mmol) and (phenyl-d5)boronic acid (3.2 g, 25.4 mmol) were added to 300 ml of THF, and the mixture was stirred and refluxed. Then, potassium carbonate (10 g, 72.7 mmol) was dissolved in 30 ml of water and added thereto, and the mixture was sufficiently stirred and then bis(tri-tert-butylphosphine)palladium(0) (0.1 g, 0.2 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 11.1 g of Compound 1-42. (Yield: 69%, MS: [M+H]⁺=666).

### Synthesis Example 1-43

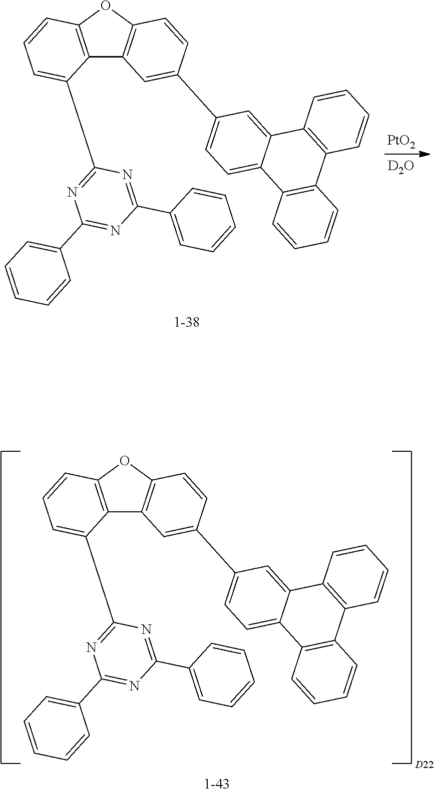

Compound 1-38 (10 g, 16 mmol), PtO₂ (1.1 g, 4.8 mmol) and D₂O (80 ml) were added to a shaker tube, and then the tube was sealed and heated at 250° C. and 600 psi for 12 hours. When the reaction was completed, chloroform was added, and the reaction solution was transferred to a separatory funnel, and extracted. The extract was dried over MgSO₄ and concentrated, and then the sample was purified by silica gel column chromatography to prepare 3.5 g of Compound 1-43. (Yield: 34%, MS: [M+H]⁺=649)

### Synthesis Example 1-44

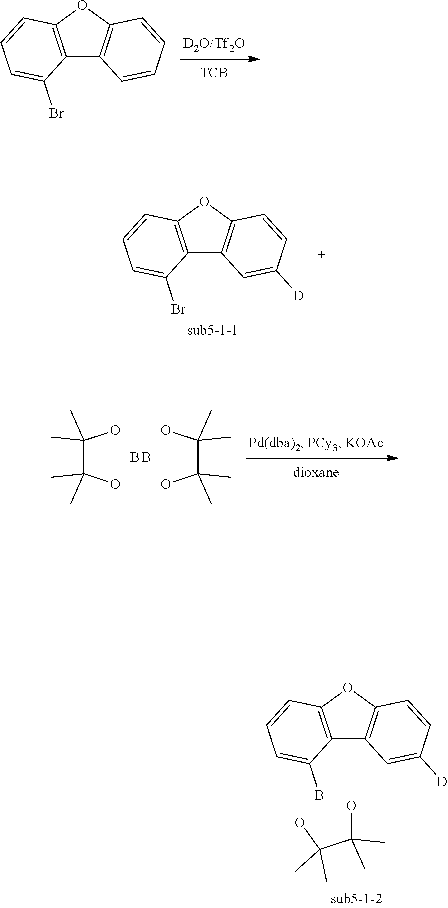

Trifluoromethanesulfonic anhydride (24 g, 85 mmol) and deuterium oxide (8.5 g, 424.9 mmol) were added at 0° C. and stirred for 5 hours to prepare a solution. 1-Bromodibenzo[b,d]furan (15 g, 60.7 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of 1-bromodibenzo[b,d]furan and 1,2,4-trichlorobenzene, and the mixture was stirred while heating up to 140° C. and then keeping that temperature. After the reaction for 5 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 5.7 g of compound Sub5-1-1. (Yield: 38%, MS: [M+H]⁺=248)

Compound Sub5-1-1 (15 g, 60.5 mmol) and bis(pinacolato)diboron (16.9 g, 66.5 mmol) were added to 300 ml of 1,4-dioxane, and the mixture was stirred under reflux. Then, potassium acetate (8.9 g, 90.7 mmol) was added thereto, the mixture was sufficiently stirred, and then bis(dibenzylideneacetone) palladium(0) (1 g, 1.8 mmol) and tricyclohexylphosphine (1 g, 3.6 mmol) were added. After the reaction for 6 hours, the reaction mixture was cooled to room temperature, the organic layer was separated using chloroform and water, and then the organic layer was distilled. This was dissolved again in chloroform, washed twice with water, and the organic layer was then separated. Anhydrous magnesium sulfate was added thereto, stirred, then filtered, and the filtrate was distilled under reduced pressure. The concentrated compound was purified by silica gel column chromatography to prepare 13.4 g of Compound Sub5-1-2. (Yield: 75%, MS: [M+H]⁺=296)

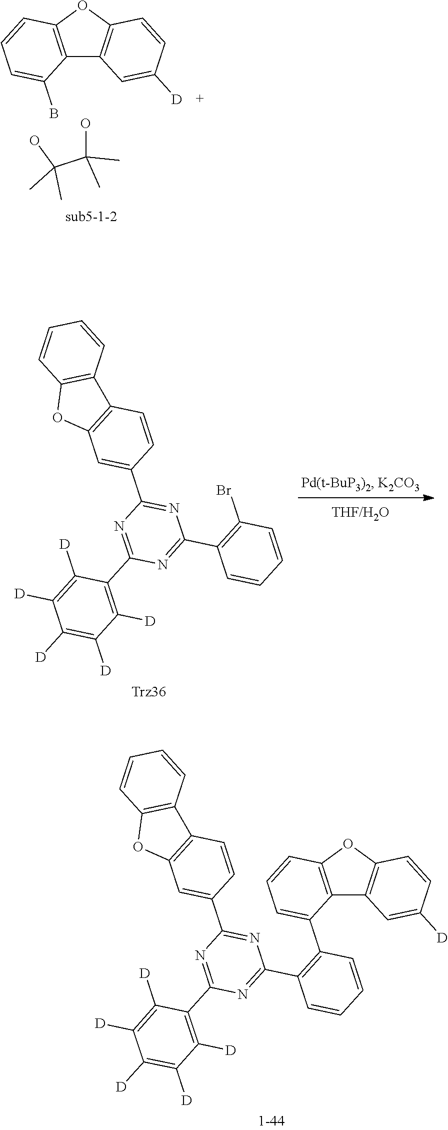

Compound Sub5-1-2 (15 g, 50.8 mmol) and Compound Trz36 (25.8 g, 53.4 mmol) were added to 300 ml of THF, and the mixture was stirred and refluxed. Then, potassium carbonate (21.1 g, 152.5 mmol) was dissolved in 63 ml of water and added thereto, and the mixture was sufficiently stirred and then bis(tri-tert-butylphosphine)palladium(0) (0.3 g, 0.5 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 18.9 g of Compound 1-44. (Yield: 72%, MS: [M+H]⁺=518).

### Synthesis Example 1-45

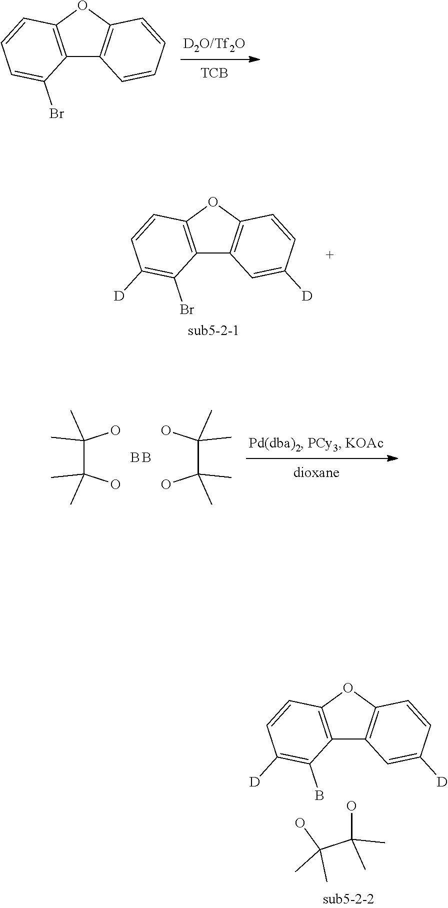

Trifluoromethanesulfonic anhydride (48 g, 170 mmol) and deuterium oxide (17 g, 849.9 mmol) were added at 0° C. and stirred for 5 hours to prepare a solution. 1-Bromodibenzo[b,d]furan (15 g, 60.7 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of 1-bromodibenzo[b,d]furan and 1,2,4-trichlorobenzene, and the mixture was stirred while heating up to 140° C. and then keeping that temperature. After the reaction for 8 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 6 g of compound Sub5-2-1. (Yield: 40%, MS: [M+H]⁺=249)

Compound Sub5-2-1 (15 g, 60.2 mmol) and bis(pinacolato)diboron (16.8 g, 66.2 mmol) were added to 300 ml of 1,4-dioxane, and the mixture was stirred under reflux. Then, potassium acetate (8.9 g, 90.3 mmol) was added thereto, the mixture was sufficiently stirred, and then bis(dibenzylideneacetone) palladium(0) (1 g, 1.8 mmol) and tricyclohexylphosphine (1 g, 3.6 mmol) were added. After the reaction for 4 hours, the reaction mixture was cooled to room temperature, the organic layer was separated using chloroform and water, and then the organic layer was distilled. This was dissolved again in chloroform, washed twice with water, and the organic layer was then separated. Anhydrous magnesium sulfate was added thereto, stirred, then filtered, and the filtrate was distilled under reduced pressure. The concentrated compound was purified by silica gel column chromatography to prepare 12.5 g of Compound Sub5-2-2. (Yield: 70%, MS: [M+H]⁺=297)

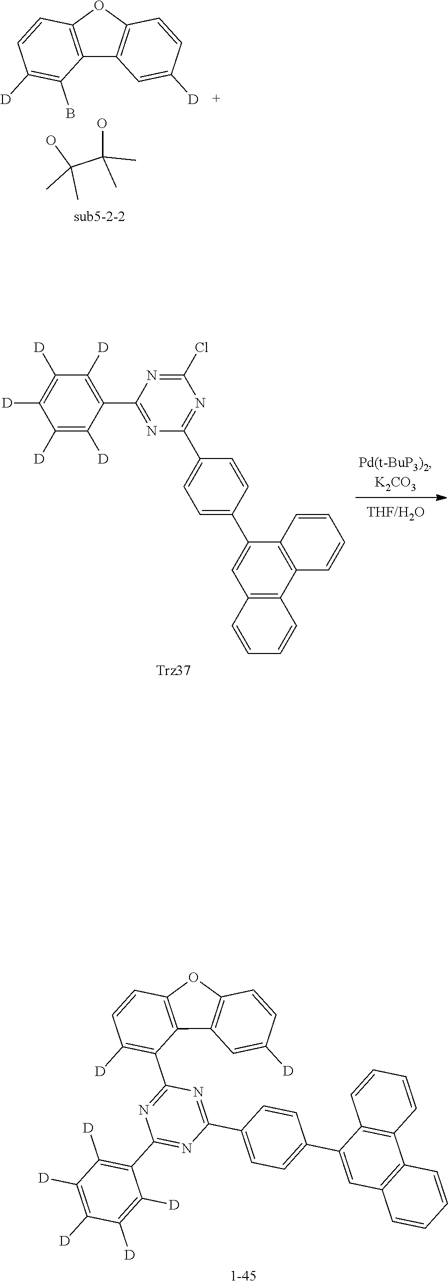

Compound Sub5-2-2 (15 g, 50.6 mmol) and Compound Trz37 (23.9 g, 53.2 mmol) were added to 300 ml of THF, and the mixture was stirred and refluxed. Then, potassium carbonate (21 g, 151.9 mmol) was dissolved in 63 ml of water and added thereto, and the mixture was sufficiently stirred and then bis(tri-tert-butylphosphine)palladium(0) (0.3 g, 0.5 mmol) was added. After the reaction for 2 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 19.5 g of Compound 1-45. (Yield: 66%, MS: [M+H]⁺=583).

### Synthesis Example 1-46

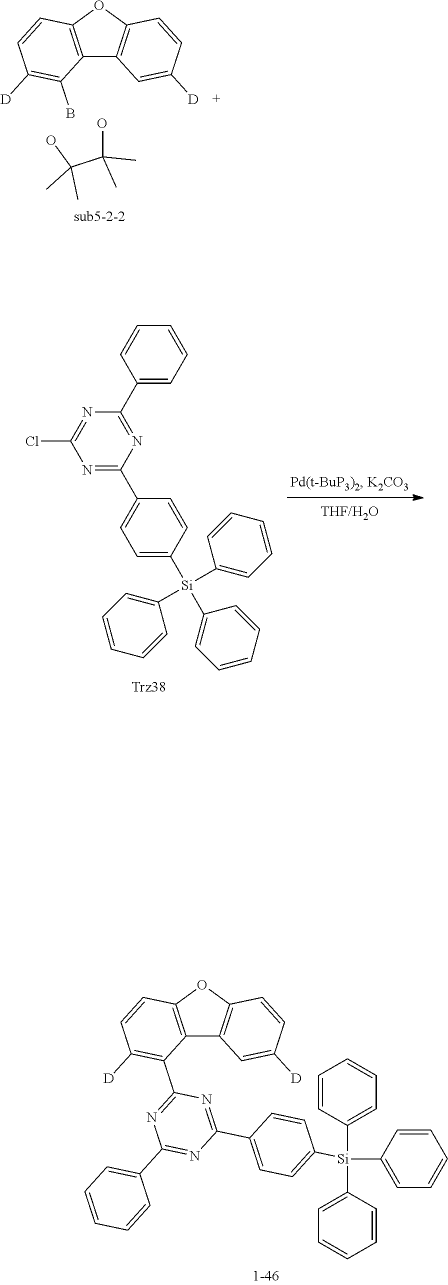

Compound Sub5-2-2 (15 g, 50.6 mmol) and Compound Trz38 (28 g, 53.2 mmol) were added to 300 ml of THF, and the mixture was stirred and refluxed. Then, potassium carbonate (21 g, 151.9 mmol) was dissolved in 63 ml of water and added thereto, and the mixture was sufficiently stirred and then bis(tri-tert-butylphosphine)palladium(0) (0.3 g, 0.5 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 21.4 g of Compound 1-46. (Yield: 64%, MS: [M+H]⁺=660).

### Synthesis Example 1-47

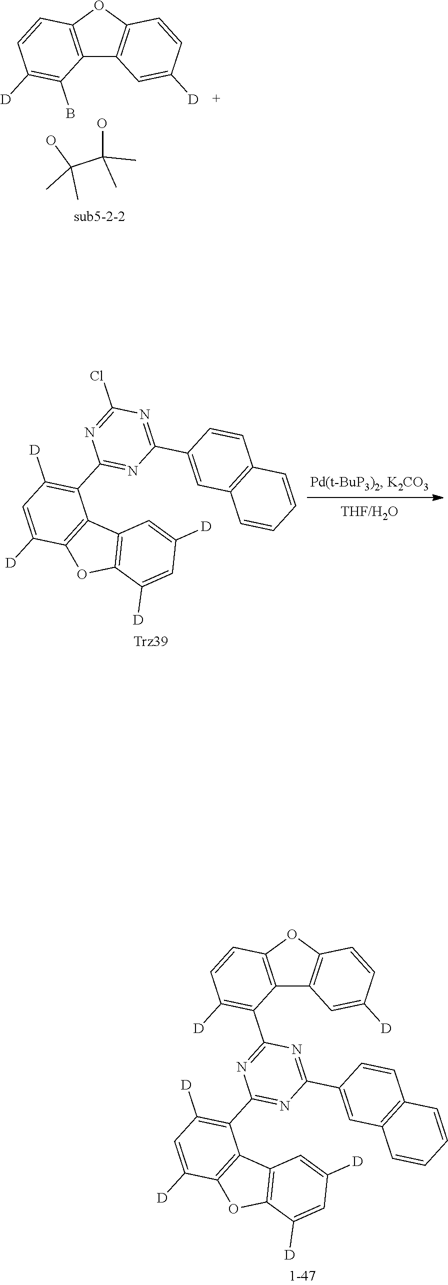

Compound Sub5-2-2 (15 g, 50.6 mmol) and Compound Trz39 (21.9 g, 53.2 mmol) were added to 300 ml of THF, and the mixture was stirred and refluxed. Then, potassium carbonate (21 g, 151.9 mmol) was dissolved in 63 ml of water and added thereto, and the mixture was sufficiently stirred and then bis(tri-tert-butylphosphine)palladium(0) (0.3 g, 0.5 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 19.1 g of Compound 1-47. (Yield: 69%, MS: [M+H]⁺=546).

### Synthesis Example 1-48

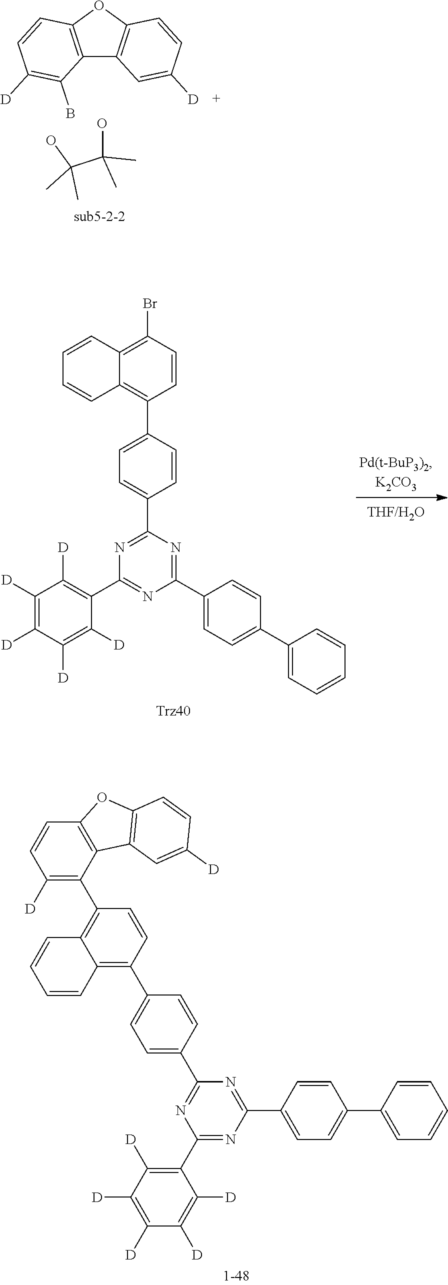

Compound Sub5-2-2 (15 g, 50.6 mmol) and Compound Trz40 (31.7 g, 53.2 mmol) were added to 300 ml of THF, and the mixture was stirred and refluxed. Then, potassium carbonate (21 g, 151.9 mmol) was dissolved in 63 ml of water and added thereto, and the mixture was sufficiently stirred and then bis(tri-tert-butylphosphine)palladium(0) (0.3 g, 0.5 mmol) was added. After the reaction for 2 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 23.9 g of Compound 1-48. (Yield: 69%, MS: [M+H]⁺=685).

### Synthesis Example 1-49

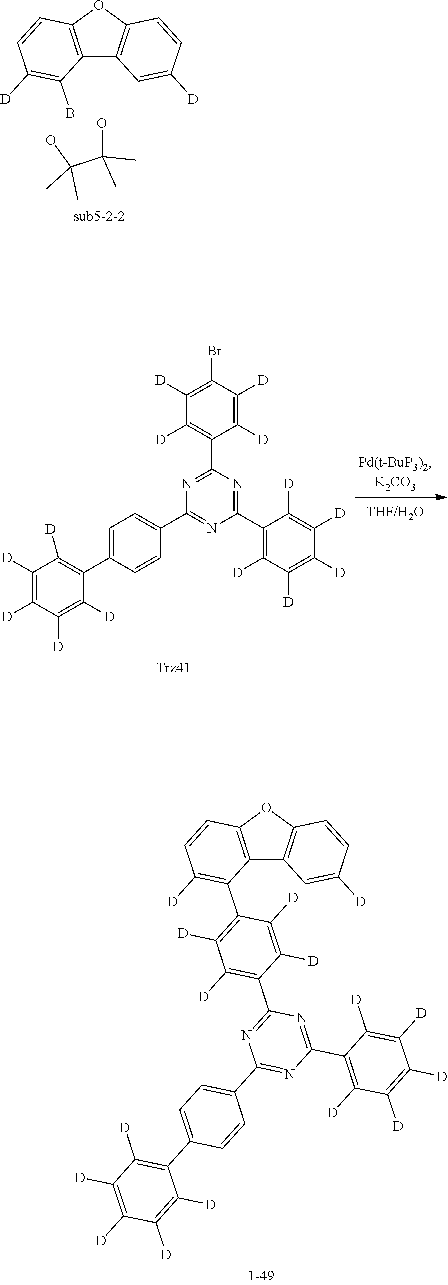

Compound Sub5-2-2 (15 g, 50.6 mmol) and Compound Trz41 (25.4 g, 53.2 mmol) were added to 300 ml of THF, and the mixture was stirred and refluxed. Then, potassium carbonate (21 g, 151.9 mmol) was dissolved in 63 ml of water and added thereto, and the mixture was sufficiently stirred and then bis(tri-tert-butylphosphine)palladium(0) (0.3 g, 0.5 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 21.5 g of Compound 1-49. (Yield: 75%, MS: [M+H]⁺=568).

### Synthesis Example 1-50

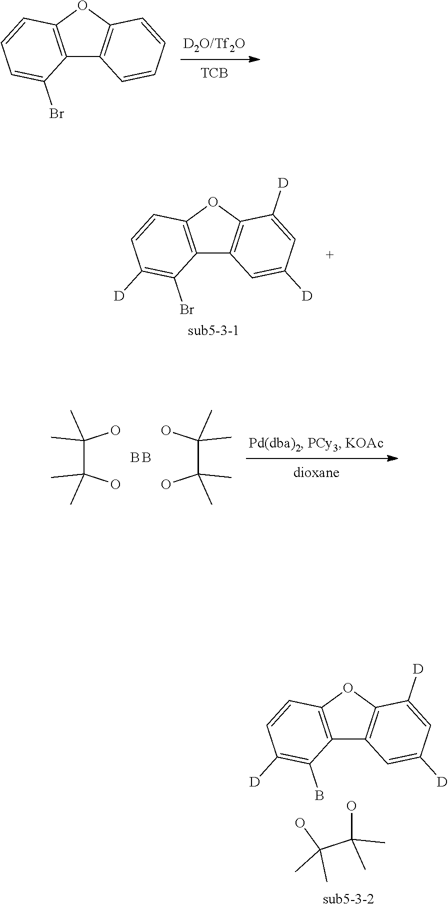

Trifluoromethanesulfonic anhydride (71.9 g, 255 mmol) and deuterium oxide (25.5 g, 1274.8 mmol) were added at 0° C. and stirred for hours to prepare a solution. 1-bromodibenzo[b,d]furan (15 g, 60.7 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of 1-bromodibenzo[b,d]furan and 1,2,4-trichlorobenzene, and the mixture was stirred while heating up to 140° C. and then keeping that temperature. After the reaction for 14 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 6.3 g of compound Sub5-3-1. (Yield: 42%, MS: [M+H]⁺=250)

Compound Sub5-3-1 (15 g, 60 mmol) and bis(pinacolato)diboron (16.8 g, 66 mmol) were added to 300 ml of 1,4-dioxane, and the mixture was stirred under reflux. Then, potassium acetate (8.8 g, 90 mmol) was added thereto, the mixture was sufficiently stirred, and then bis(dibenzylideneacetone) palladium(0) (1 g, 1.8 mmol) and tricyclohexylphosphine (1 g, 3.6 mmol) were added. After the reaction for 6 hours, the reaction mixture was cooled to room temperature, the organic layer was separated using chloroform and water, and then the organic layer was distilled. This was dissolved again in chloroform, washed twice with water, and the organic layer was then separated. Anhydrous magnesium sulfate was added thereto, stirred, then filtered, and the filtrate was distilled under reduced pressure. The concentrated compound was purified by silica gel column chromatography to prepare 11.4 g of Compound Sub5-3-2. (Yield: 64%, MS: [M+H]⁺=298)

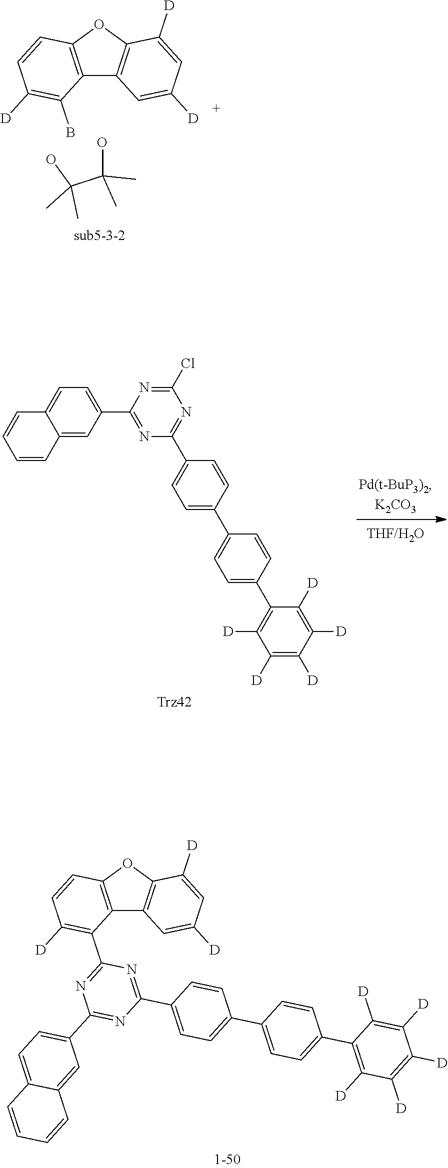

Compound Sub5-3-2 (15 g, 50.5 mmol) and Compound Trz42 (25.2 g, 53 mmol) were added to 300 ml of THF, and the mixture was stirred and refluxed. Then, potassium carbonate (20.9 g, 151.4 mmol) was dissolved in 63 ml of water and added thereto, and the mixture was sufficiently stirred and then bis(tri-tert-butylphosphine)palladium(0) (0.3 g, 0.5 mmol) was added. After the reaction for 2 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 20.3 g of Compound 1-50. (Yield: 66%, MS: [M+H]⁺=610).

### Synthesis Example 1-51

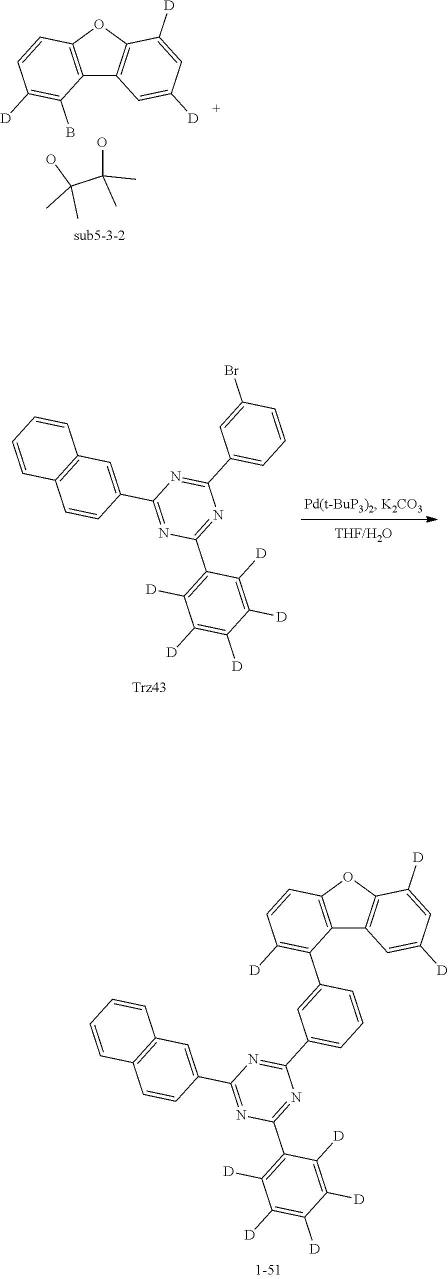

Compound Sub5-3-2 (15 g, 50.5 mmol) and Compound Trz43 (23.5 g, 53 mmol) were added to 300 ml of THF, and the mixture was stirred and refluxed. Then, potassium carbonate (20.9 g, 151.4 mmol) was dissolved in 63 ml of water and added thereto, and the mixture was sufficiently stirred and then bis(tri-tert-butylphosphine)palladium(0) (0.3 g, 0.5 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 18.6 g of Compound 1-51. (Yield: 69%, MS: [M+H]⁺=534).

### Synthesis Example 1-52

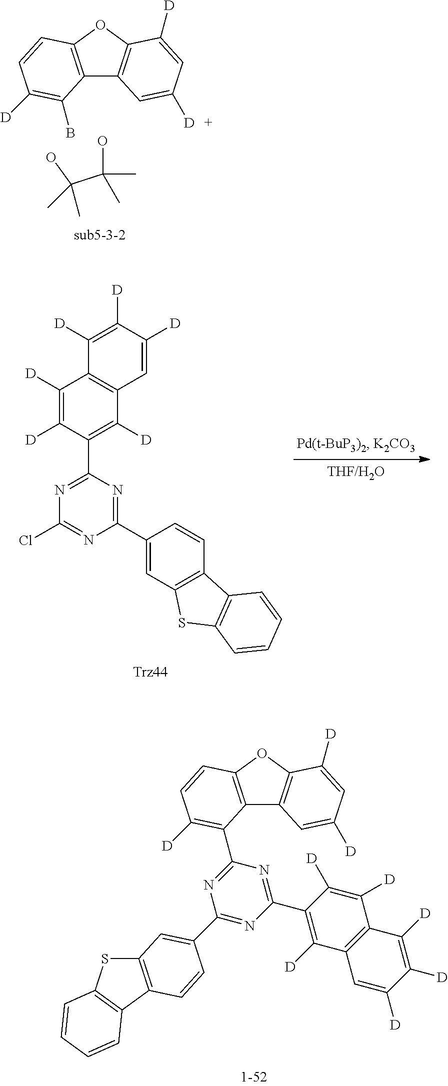

Compound Sub5-3-2 (15 g, 50.5 mmol) and Compound Trz44 (22.8 g, 53 mmol) were added to 300 ml of THF, and the mixture was stirred and refluxed. Then, potassium carbonate (20.9 g, 151.4 mmol) was dissolved in 63 ml of water and added thereto, and the mixture was sufficiently stirred and then bis(tri-tert-butylphosphine)palladium(0) (0.3 g, 0.5 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 21.1 g of Compound 1-52. (Yield: 74%, MS: [M+H]⁺=565).

### Synthesis Example 1-53

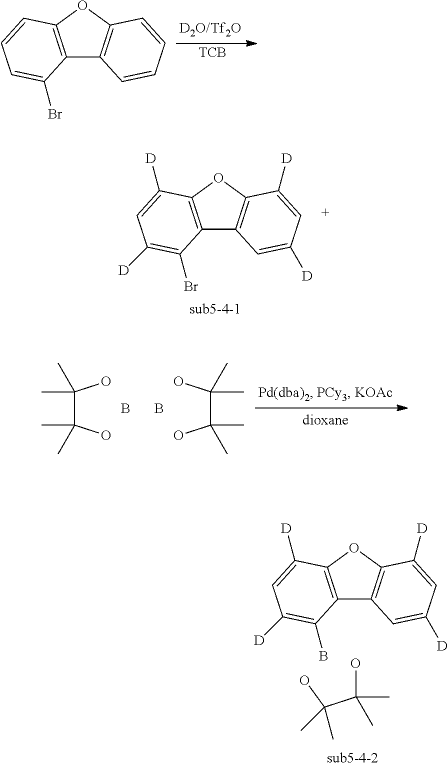

Trifluoromethanesulfonic anhydride (95.9 g, 340 mmol) and deuterium oxide (34 g, 1699.8 mmol) were added at 0° C. and stirred for 5 hours to prepare a solution. 1-bromodibenzo[b,d]furan (15 g, 60.7 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of 1-bromodibenzo[b,d]furan and 1,2,4-trichlorobenzene, and the mixture was stirred while heating up to 140° C. and then keeping that temperature. After the reaction for 20 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 5.6 g of compound Sub5-4-1. (Yield: 37%, MS: [M+H]⁺=251)

Compound Sub5-4-1 (15 g, 59.7 mmol) and bis(pinacolato)diboron (16.7 g, 65.7 mmol) were added to 300 ml of 1,4-dioxane, and the mixture was stirred under reflux. Then, potassium acetate (8.8 g, 89.6 mmol) was added thereto, the mixture was sufficiently stirred, and then bis(dibenzylideneacetone) palladium(0) (1 g, 1.8 mmol) and tricyclohexylphosphine (1 g, 3.6 mmol) were added. After the reaction for 5 hours, the reaction mixture was cooled to room temperature, the organic layer was separated using chloroform and water, and then the organic layer was distilled. This was dissolved again in chloroform, washed twice with water, and the organic layer was then separated. Anhydrous magnesium sulfate was added thereto, stirred, then filtered, and the filtrate was distilled under reduced pressure. The concentrated compound was purified by silica gel column chromatography to prepare 12.5 g of Compound Sub5-4-2. (Yield: 70%, MS: [M+H]⁺=299)

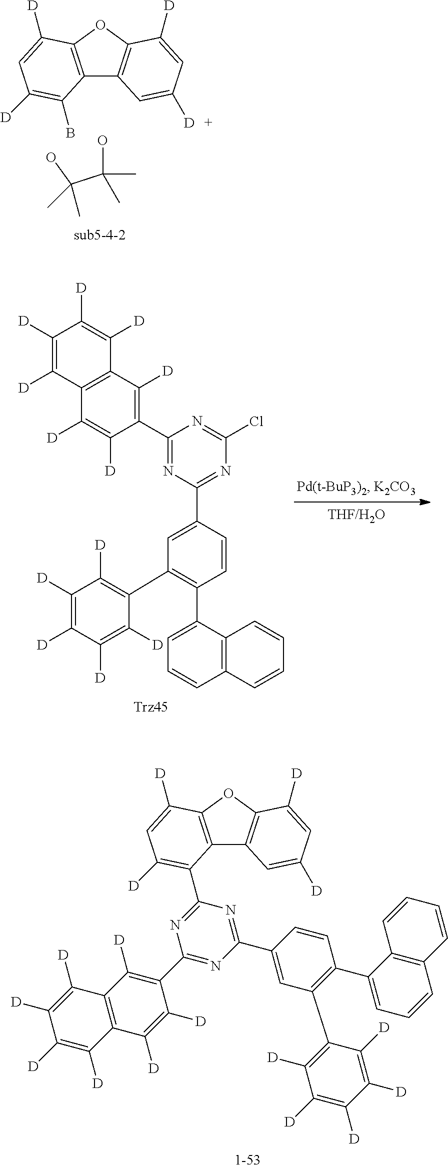

Compound Sub5-4-2 (15 g, 50.3 mmol) and Compound Trz45 (28.1 g, 52.8 mmol) were added to 300 ml of THF, and the mixture was stirred and refluxed. Then, potassium carbonate (20.9 g, 150.9 mmol) was dissolved in 63 ml of water and added thereto, and the mixture was sufficiently stirred and then bis(tri-tert-butylphosphine)palladium(0) (0.3 g, 0.5 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 23.8 g of Compound 1-53. (Yield: 71%, MS: [M+H]⁺=668).

### Synthesis Example 1-54

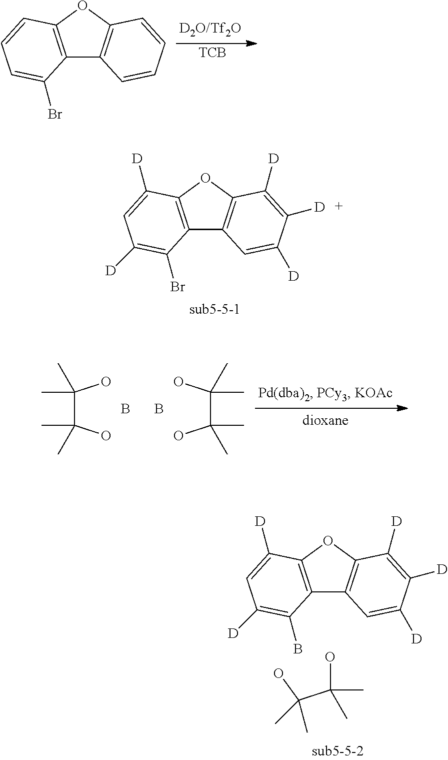

Trifluoromethanesulfonic anhydride (119.9 g, 424.9 mmol) and deuterium oxide (42.6 g, 2124.7 mmol) were added at 0° C. and stirred for hours to prepare a solution. 1-Bromodibenzo[b,d]furan (15 g, 60.7 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of 1-bromodibenzo[b,d]furan and 1,2,4-trichlorobenzene, and the mixture was stirred while heating up to 140° C. and then keeping that temperature. After the reaction for 24 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 5.9 g of compound Sub5-5-1. (Yield: 39%, MS: [M+H]⁺=252)

Compound Sub5-5-1 (15 g, 59.5 mmol) and bis(pinacolato)diboron (16.6 g, 65.4 mmol) were added to 300 ml of 1,4-dioxane, and the mixture was stirred under reflux. Then, potassium acetate (8.8 g, 89.2 mmol) was added thereto, the mixture was sufficiently stirred, and then bis(dibenzylideneacetone) palladium(0) (1 g, 1.8 mmol) and tricyclohexylphosphine (1 g, 3.6 mmol) were added. After the reaction for 4 hours, the reaction mixture was cooled to room temperature, the organic layer was separated using chloroform and water, and then the organic layer was distilled. This was dissolved again in chloroform, washed twice with water, and the organic layer was then separated. Anhydrous magnesium sulfate was added thereto, stirred, then filtered, and the filtrate was distilled under reduced pressure. The concentrated compound was purified by silica gel column chromatography to prepare 11.2 g of Compound Sub5-5-2. (Yield: 63%, MS: [M+H]⁺=300)

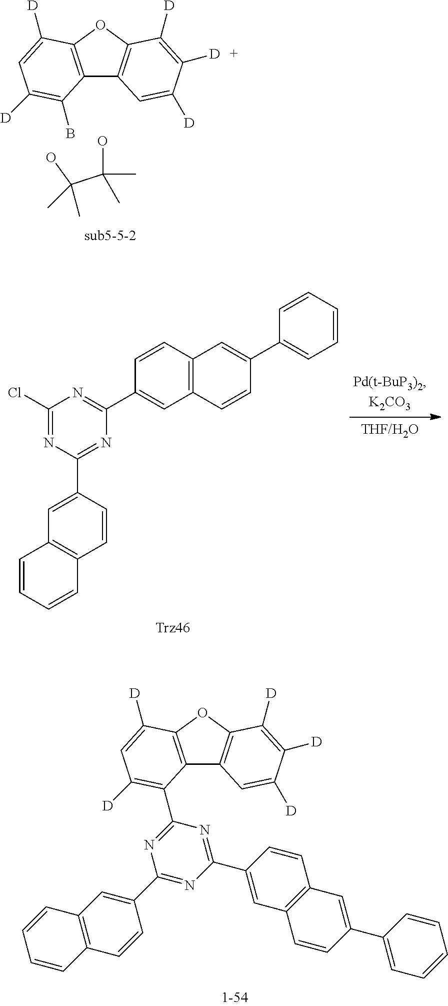

Compound Sub5-5-2 (15 g, 50.1 mmol) and Compound Trz46 (27.6 g, 52.6 mmol) were added to 300 ml of THF, and the mixture was stirred and refluxed. Then, potassium carbonate (20.8 g, 150.4 mmol) was dissolved in 62 ml of water and added thereto, and the mixture was sufficiently stirred and then bis(tri-tert-butylphosphine)palladium(0) (0.3 g, 0.5 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 21.2 g of Compound 1-54. (Yield: 73%, MS: [M+H]⁺=581).

### Synthesis Example 1-55

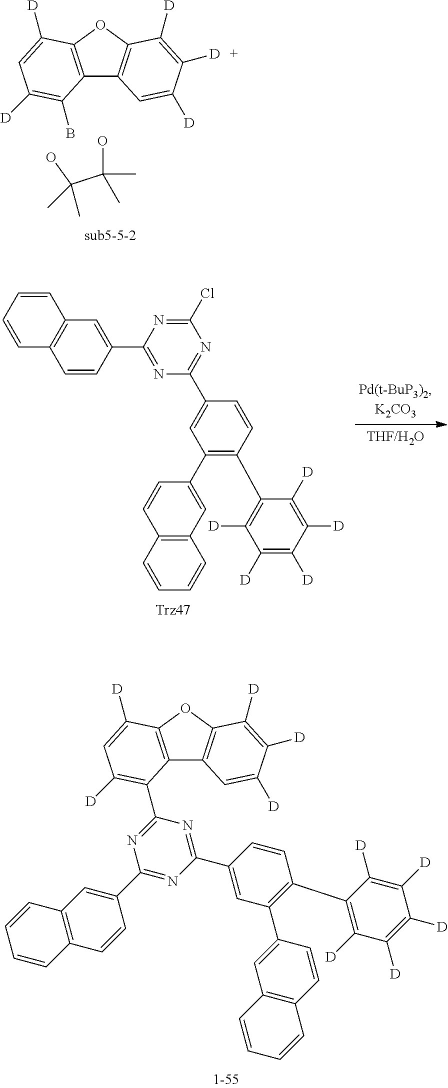

Compound Sub5-5-2 (15 g, 50.1 mmol) and Compound Trz47 (27.6 g, 52.6 mmol) were added to 300 ml of THF, and the mixture was stirred and refluxed. Then, potassium carbonate (20.8 g, 150.4 mmol) was dissolved in 62 ml of water and added thereto, and the mixture was sufficiently stirred and then bis(tri-tert-butylphosphine)palladium(0) (0.3 g, 0.5 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 24.9 g of Compound 1-55. (Yield: 75%, MS: [M+H]⁺=662).

### Synthesis Example 1-56

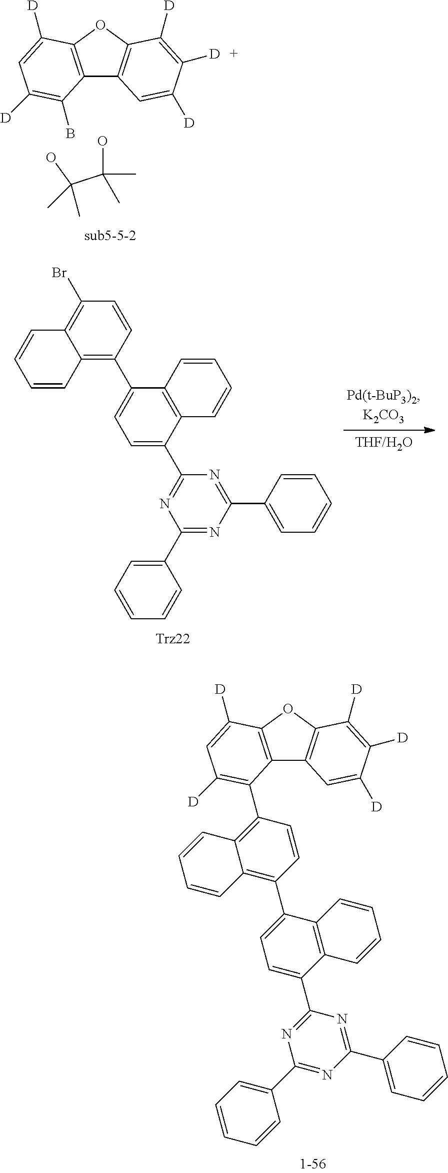

Compound Sub5-5-2 (15 g, 50.1 mmol) and Compound Trz22 (29.7 g, 52.6 mmol) were added to 300 ml of THF, and the mixture was stirred and refluxed. Then, potassium carbonate (20.8 g, 150.4 mmol) was dissolved in 62 ml of water and added thereto, and the mixture was sufficiently stirred and then bis(tri-tert-butylphosphine)palladium(0) (0.3 g, 0.5 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 23.4 g of Compound 1-56. (Yield: 71%, MS: [M+H]⁺=657).

### Synthesis Example 1-57

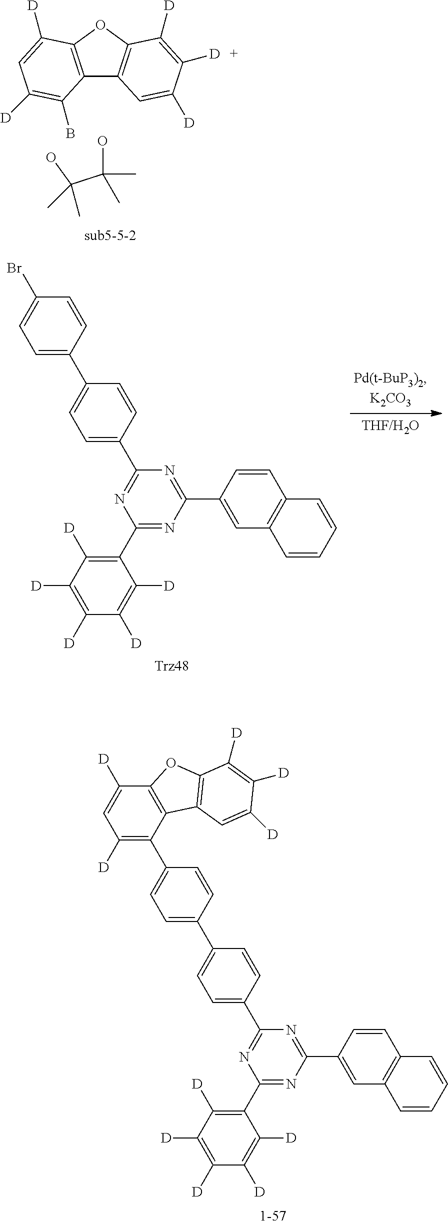

Compound Sub5-5-2 (15 g, 50.1 mmol) and Compound Trz48 (27.3 g, 52.6 mmol) were added to 300 ml of THF, and the mixture was stirred and refluxed. Then, potassium carbonate (20.8 g, 150.4 mmol) was dissolved in 62 ml of water and added thereto, and the mixture was sufficiently stirred and then bis(tri-tert-butylphosphine)palladium(0) (0.3 g, 0.5 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 19 g of Compound 1-57. (Yield: 62%, MS: [M+H]⁺=612).

### Synthesis Example 1-58

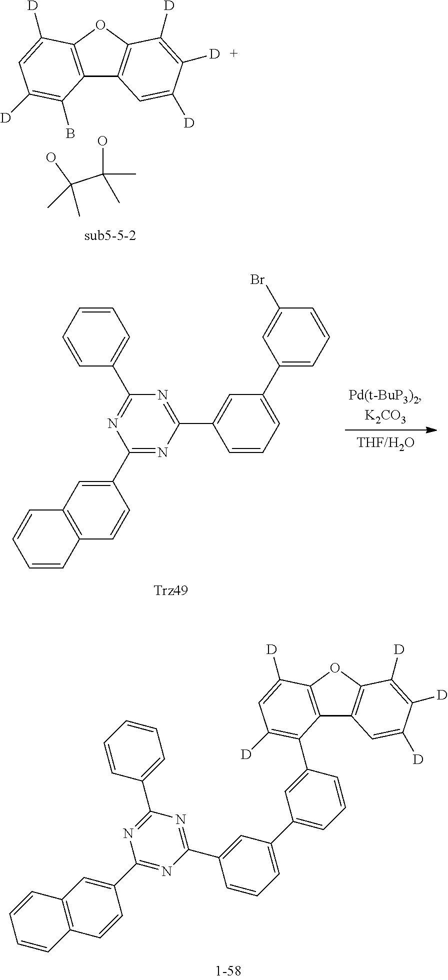

Compound Sub5-5-2 (15 g, 50.1 mmol) and Compound Trz49 (27.1 g, 52.6 mmol) were added to 300 ml of THF, and the mixture was stirred and refluxed. Then, potassium carbonate (20.8 g, 150.4 mmol) was dissolved in 62 ml of water and added thereto, and the mixture was sufficiently stirred and then bis(tri-tert-butylphosphine)palladium(0) (0.3 g, 0.5 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 19.5 g of Compound 1-58. (Yield: 64%, MS: [M+H]⁺=607).

### Synthesis Example 1-59

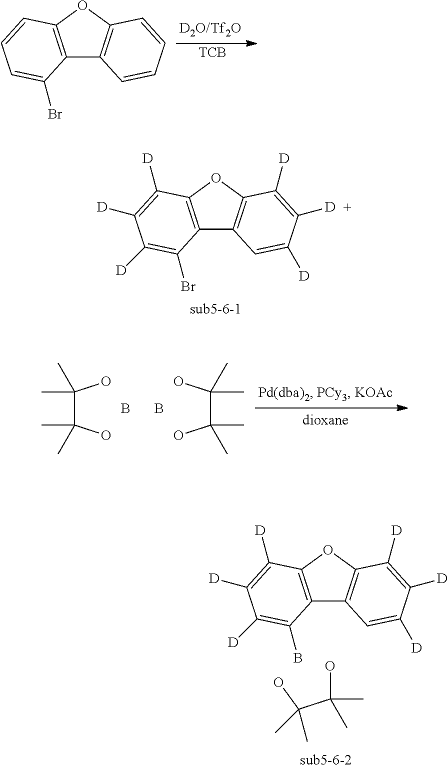

Trifluoromethanesulfonic anhydride (167.8 g, 594.9 mmol) and deuterium oxide (59.6 g, 2974.6 mmol) were added at 0° C. and stirred for hours to prepare a solution. 1-Bromodibenzo[b,d]furan (15 g, 60.7 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of 1-bromodibenzo[b,d]furan and 1,2,4-trichlorobenzene, and the mixture was stirred while heating up to 140° C. and then keeping that temperature. After the reaction for 36 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 6.1 g of compound Sub5-6-1. (Yield: 40%, MS: [M+H]⁺=254)

Compound Sub5-6-1 (15 g, 59 mmol) and bis(pinacolato)diboron (16.5 g, 64.9 mmol) were added to 300 ml of 1,4-dioxane, and the mixture was stirred under reflux. Then, potassium acetate (8.7 g, 88.5 mmol) was added thereto, the mixture was sufficiently stirred, and then bis(dibenzylideneacetone) palladium(0) (1 g, 1.8 mmol) and tricyclohexylphosphine (1 g, 3.5 mmol) were added. After the reaction for 4 hours, the reaction mixture was cooled to room temperature, the organic layer was separated using chloroform and water, and then the organic layer was distilled. This was dissolved again in chloroform, washed twice with water, and the organic layer was then separated. Anhydrous magnesium sulfate was added thereto, stirred, then filtered, and the filtrate was distilled under reduced pressure. The concentrated compound was purified by silica gel column chromatography to prepare 11.6 g of Compound Sub5-6-2. (Yield: 65%, MS: [M+H]⁺=302)

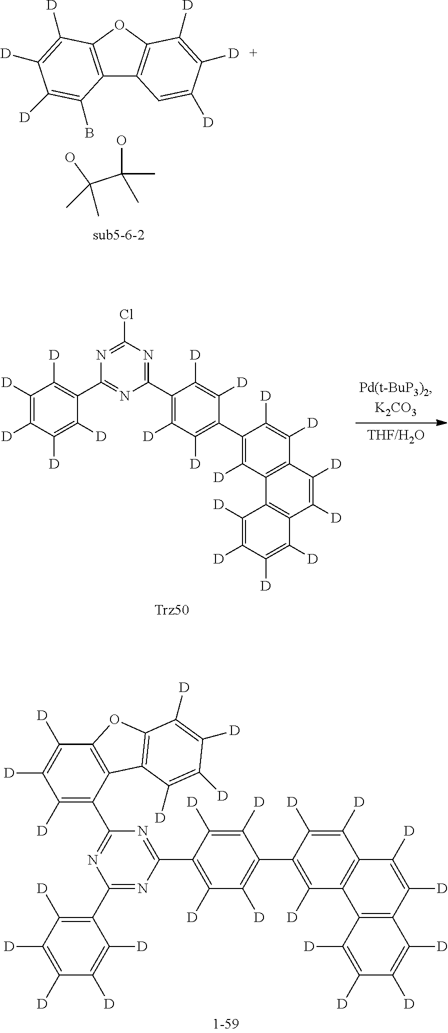

Compound Sub5-6-2 (15 g, 50 mmol) and Compound Trz50 (24.2 g, 52.5 mmol) were added to 300 ml of THF, and the mixture was stirred and refluxed. Then, potassium carbonate (20.7 g, 149.9 mmol) was dissolved in 62 ml of water and added thereto, and the mixture was sufficiently stirred and then bis(tri-tert-butylphosphine)palladium(0) (0.3 g, 0.5 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 22.5 g of Compound 1-59. (Yield: 75%, MS: [M+H]⁺=601).

### Synthesis Example 1-60

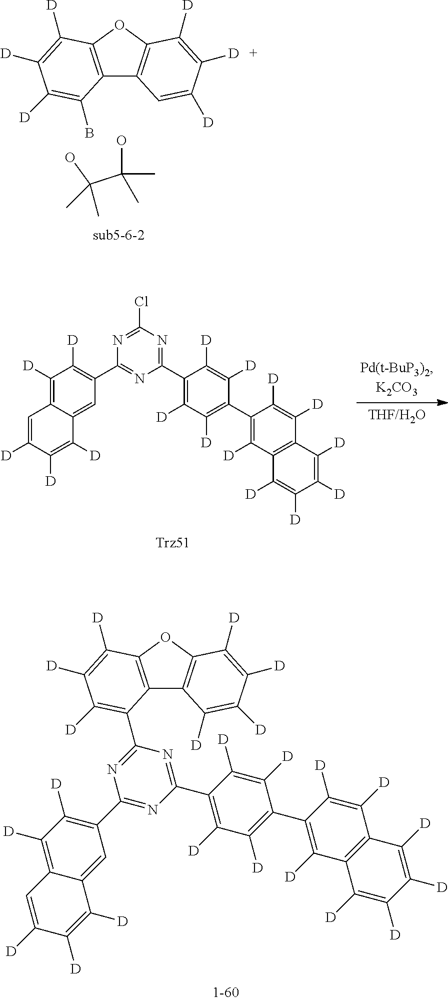

Compound Sub5-6-2 (15 g, 50 mmol) and Compound Trz51 (24.1 g, 52.5 mmol) were added to 300 ml of THF, and the mixture was stirred and refluxed. Then, potassium carbonate (20.7 g, 149.9 mmol) was dissolved in 62 ml of water and added thereto, and the mixture was sufficiently stirred and then bis(tri-tert-butylphosphine)palladium(0) (0.3 g, 0.5 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 22.1 g of Compound 1-60. (Yield: 74%, MS: [M+H]⁺=599).

### Synthesis Example 1-61

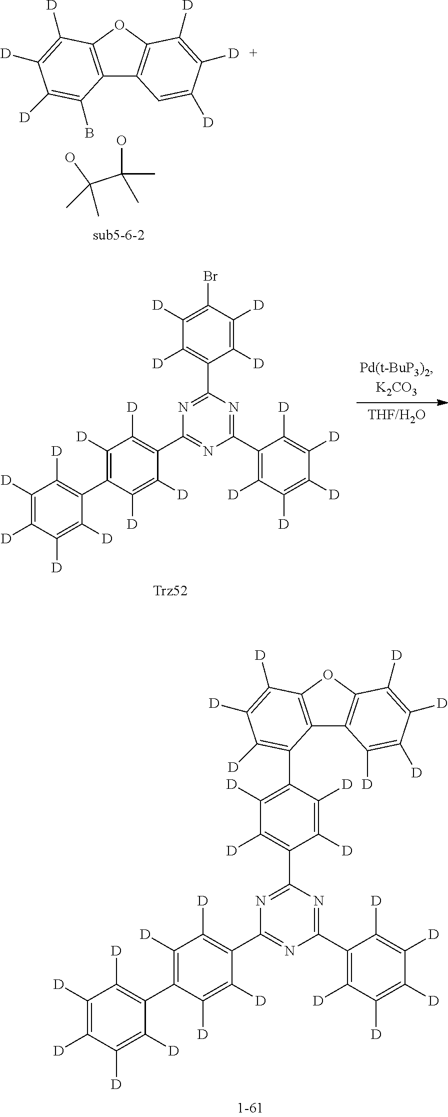

Compound Sub5-6-2 (15 g, 50 mmol) and Compound Trz52 (25.3 g, 52.5 mmol) were added to 300 ml of THF, and the mixture was stirred and refluxed. Then, potassium carbonate (20.7 g, 149.9 mmol) was dissolved in 62 ml of water and added thereto, and the mixture was sufficiently stirred and then bis(tri-tert-butylphosphine)palladium(0) (0.3 g, 0.5 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 21.6 g of Compound 1-61. (Yield: 75%, MS: [M+H]⁺=577).

### Synthesis Example 1-62

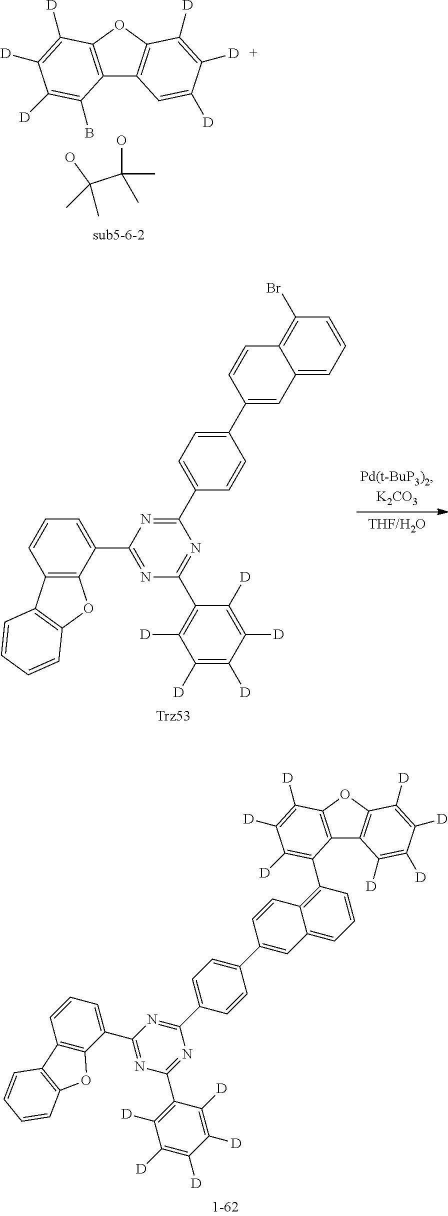

Compound Sub5-6-2 (15 g, 50 mmol) and Compound Trz53 (32 g, 52.5 mmol) were added to 300 ml of THF, and the mixture was stirred and refluxed. Then, potassium carbonate (20.7 g, 149.9 mmol) was dissolved in 62 ml of water and added thereto, and the mixture was sufficiently stirred and then bis(tri-tert-butylphosphine)palladium(0) (0.3 g, 0.5 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 26 g of Compound 1-62. (Yield: 74%, MS: [M+H]⁺=704).

### Synthesis Example 1-63

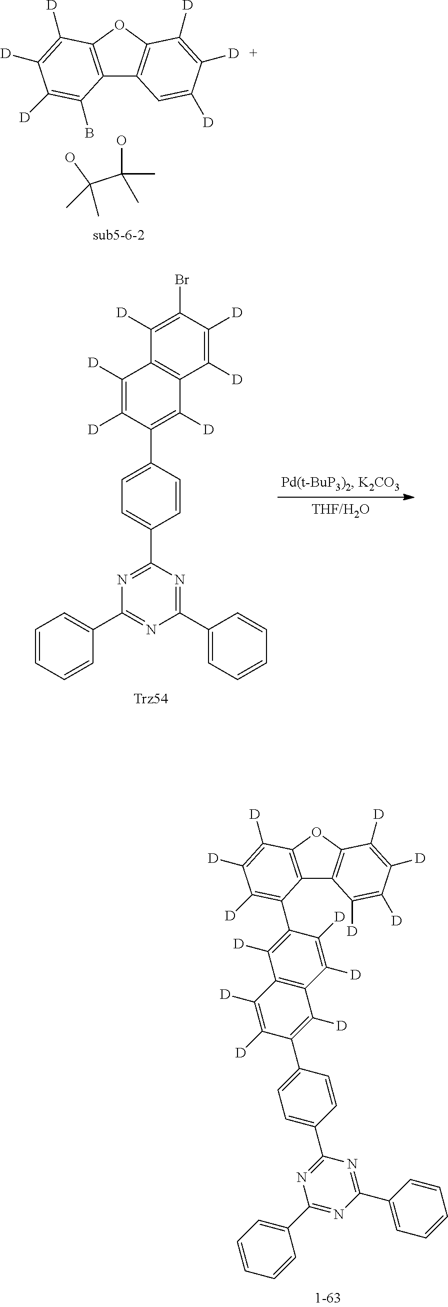

Compound Sub5-6-2 (15 g, 50 mmol) and Compound Trz54 (27.3 g, 52.5 mmol) were added to 300 ml of THF, and the mixture was stirred and refluxed. Then, potassium carbonate (20.7 g, 149.9 mmol) was dissolved in 62 ml of water and added thereto, and the mixture was sufficiently stirred and then bis(tri-tert-butylphosphine)palladium(0) (0.3 g, 0.5 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 18.4 g of Compound 1-63. (Yield: 60%, MS: [M+H]⁺=615).

### Synthesis Example 1-64

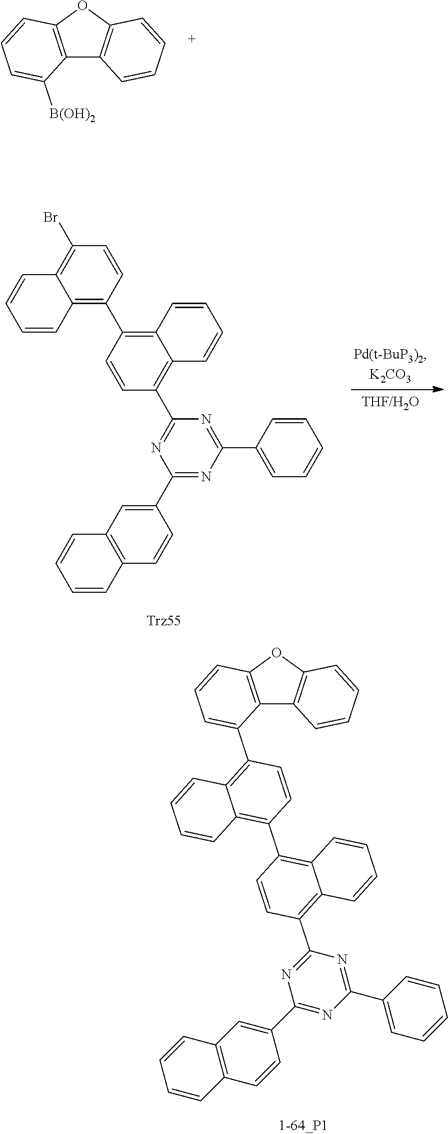

Dibenzo[b,d]furan-1-ylboronic acid (15 g, 70.8 mmol) and Compound Trz55 (45.7 g, 74.3 mmol) were added to 300 ml of THF, and the mixture was stirred and refluxed. Then, potassium carbonate (29.3 g, 212.3 mmol) was dissolved in 88 ml of water and added thereto, and the mixture was sufficiently stirred and then bis(tri-tert-butylphosphine)palladium(0) (0.4 g, 0.7 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 32.2 g of Compound 1-64_P1. (Yield: 65%, MS: [M+H]⁺=702).

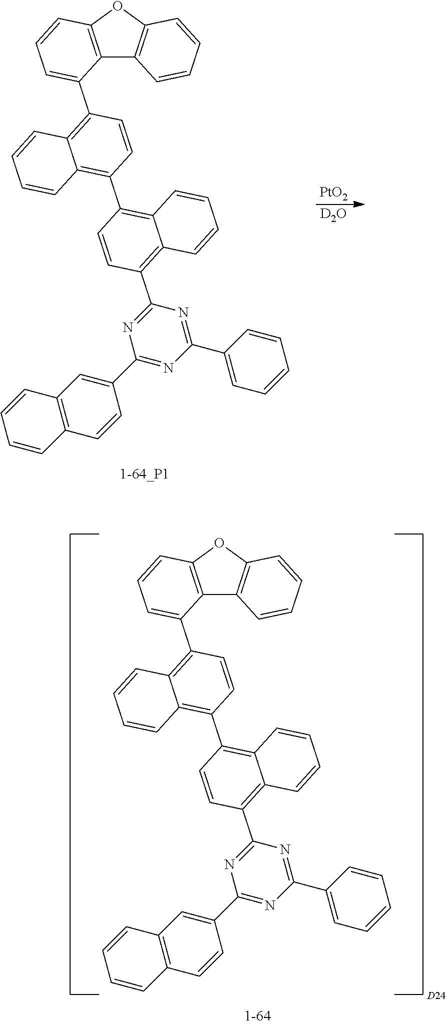

Compound 1-64_P1 (10 g, 14.2 mmol), PtO₂ (1 g, 4.3 mmol) and D₂O (71 ml) were added to a shaker tube, and then the tube was sealed and heated at 250° C. and 600 psi for 12 hours. When the reaction was completed, chloroform was added, and the reaction solution was transferred to a separatory funnel, and extracted. The extract was dried over MgSO₄ and concentrated, and then the sample was purified by silica gel column chromatography to prepare 4 g of Compound 1-64. (Yield: 39%, MS: [M+H]⁺=727)

### Synthesis Example 1-65

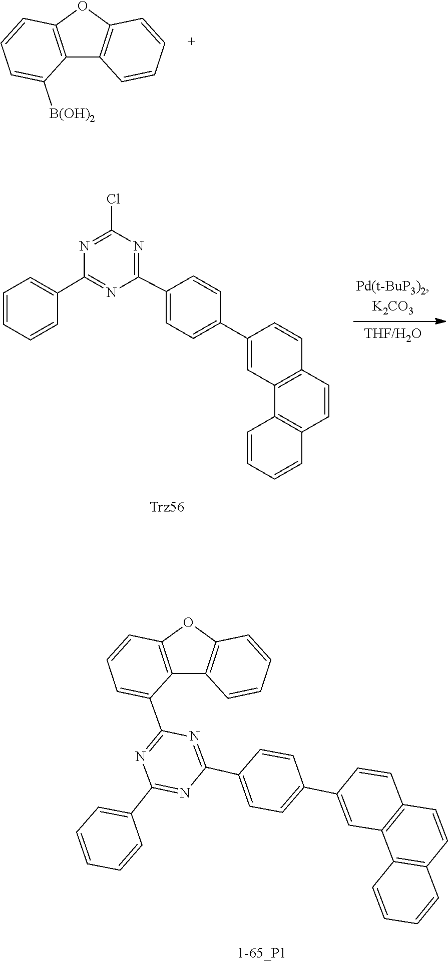 [SMILES: OB(O)c1cccc2oc3ccccc3c12 ; Clc1nc(-c2ccccc2)nc(-c2ccc(-c3ccc4ccc5ccccc5c4c3)cc2)n1 ; c1ccc(-c2nc(-c3ccc(-c4ccc5ccc6ccccc6c5c4)cc3)nc(-c3cccc4oc5ccccc5c34)n2)cc1]

Dibenzo[b,d]furan-1-ylboronic acid (15 g, 70.8 mmol) and Compound Trz56 (33 g, 74.3 mmol) were added to 300 ml of THF, and the mixture was stirred and refluxed. Then, potassium carbonate (29.3 g, 212.3 mmol) was dissolved in 88 ml of water and added thereto, and the mixture was sufficiently stirred and then bis(tri-tert-butylphosphine)palladium(0) (0.4 g, 0.7 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 29.7 g of Compound 1-65_P1. (Yield: 73%, MS: [M+H]⁺=576).

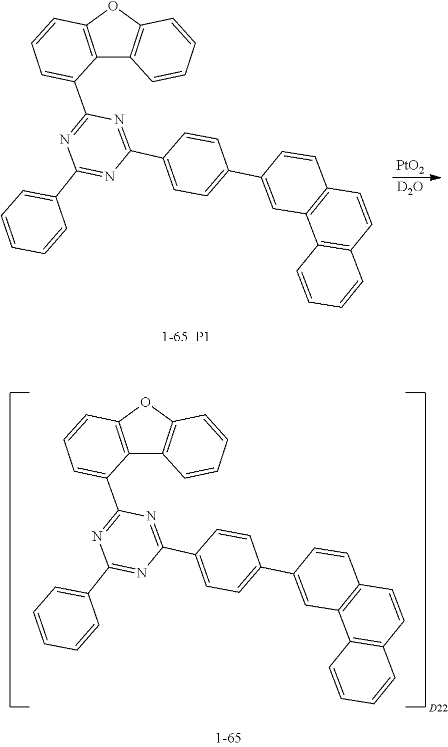

Compound 1-65_P1 (10 g, 17.4 mmol), PtO₂ (1.2 g, 5.2 mmol) and D₂O (87 ml) were added to a shaker tube, and then the tube was sealed and heated at 250° C. and 600 psi for 12 hours. When the reaction was completed, chloroform was added, and the reaction solution was transferred to a separatory funnel, and extracted. The extract was dried over MgSO₄ and concentrated, and then the sample was purified by silica gel column chromatography to prepare 5.1 g of Compound 1-65. (Yield: 49%, MS: [M+H]⁺=599)

### Synthesis Example 1-66

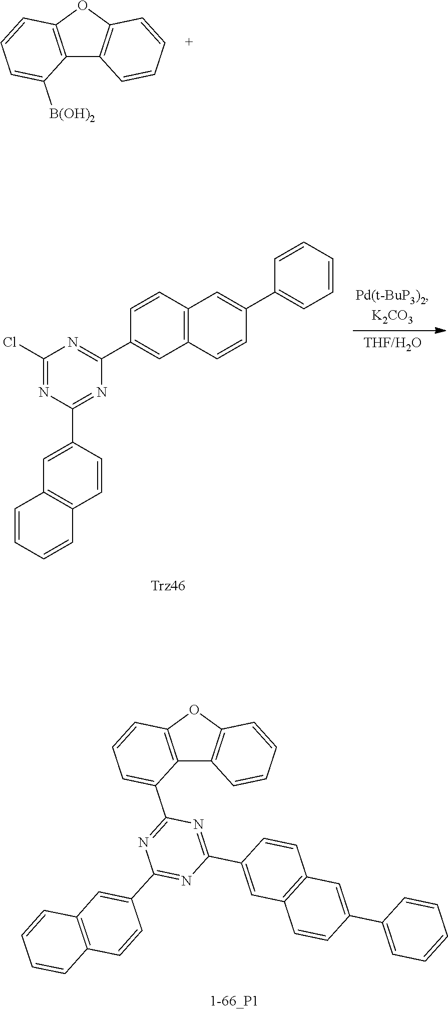 [SMILES: OB(O)c1cccc2oc3ccccc3c12 ; Clc1nc(-c2ccc3ccccc3c2)nc(-c2ccc3cc(-c4ccccc4)ccc3c2)n1 ; c1ccc(-c2ccc3cc(-c4nc(-c5ccc6ccccc6c5)nc(-c5cccc6oc7ccccc7c56)n4)ccc3c2)cc1]

Dibenzo[b,d]furan-1-ylboronic acid (15 g, 70.8 mmol) and Compound Trz46 (33 g, 74.3 mmol) were added to 300 ml of THF, and the mixture was stirred and refluxed. Then, potassium carbonate (29.3 g, 212.3 mmol) was dissolved in 88 ml of water and added thereto, and the mixture was sufficiently stirred and then bis(tri-tert-butylphosphine)palladium(0) (0.4 g, 0.7 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 24.8 g of Compound 1-66_P1. (Yield: 61%, MS: [M+H]⁺=576).

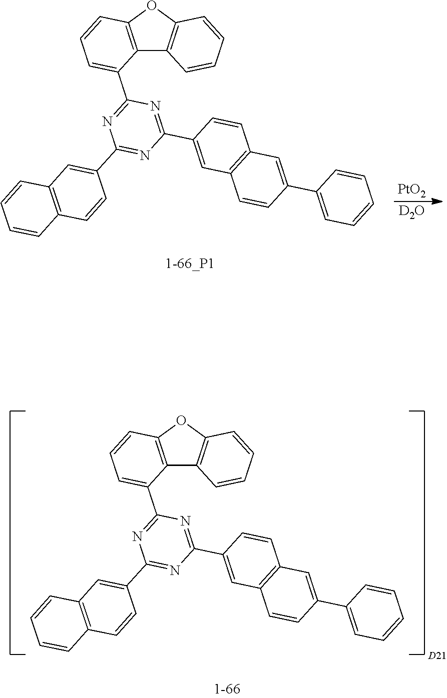

Compound 1-66_P1 (10 g, 17.4 mmol), PtO₂ (1.2 g, 5.2 mmol) and D₂O (87 ml) were added to a shaker tube, and then the tube was sealed and heated at 250° C. and 600 psi for 12 hours. When the reaction was completed, chloroform was added, and the reaction solution was transferred to a separatory funnel, and extracted. The extract was dried over MgSO₄ and concentrated, and then the sample was purified by silica gel column chromatography to prepare 5 g of Compound 1-66. (Yield: 48%, MS: [M+H]⁺=598)

### Synthesis Example 1-67

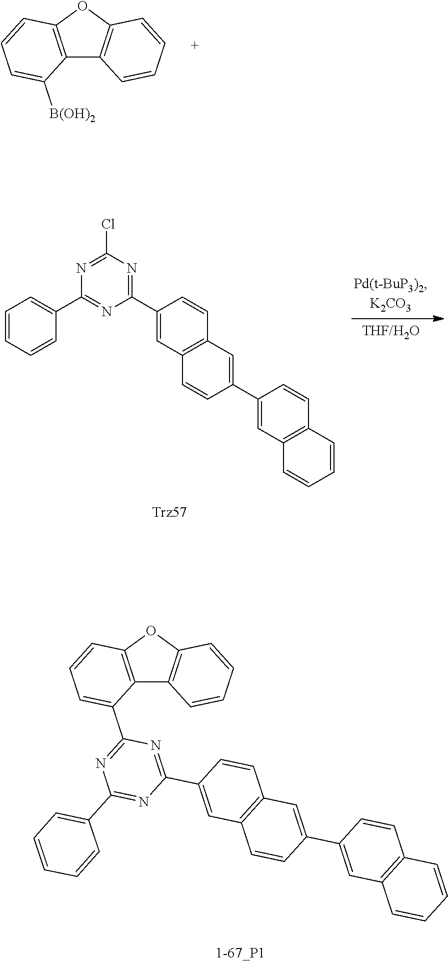 [SMILES: OB(O)c1cccc2oc3ccccc3c12 ; Clc1nc(-c2ccccc2)nc(-c2ccc3cc(-c4ccc5ccccc5c4)ccc3c2)n1 ; c1ccc(-c2nc(-c3ccc4cc(-c5ccc6ccccc6c5)ccc4c3)nc(-c3cccc4oc5ccccc5c34)n2)cc1]

Dibenzo[b,d]furan-1-ylboronic acid (15 g, 70.8 mmol) and Compound Trz57 (33 g, 74.3 mmol) were added to 300 ml of THF, and the mixture was stirred and refluxed. Then, potassium carbonate (29.3 g, 212.3 mmol) was dissolved in 88 ml of water and added thereto, and the mixture was sufficiently stirred and then bis(tri-tert-butylphosphine)palladium(0) (0.4 g, 0.7 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 26.5 g of Compound 1-67_P1. (Yield: 65%, MS: [M+H]⁺=576).

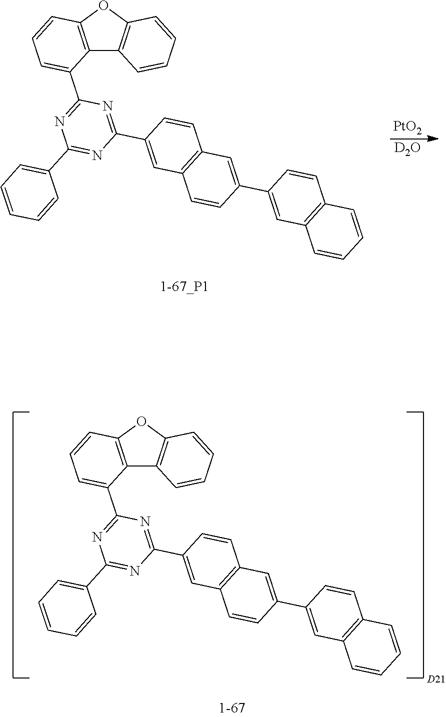

Compound 1-67_P1 (10 g, 17.4 mmol), PtO₂ (1.2 g, 5.2 mmol) and D₂O (87 ml) were added to a shaker tube, and then the tube was sealed and heated at 250° C. and 600 psi for 12 hours. When the reaction was completed, chloroform was added, and the reaction solution was transferred to a separatory funnel, and extracted. The extract was dried over MgSO₄ and concentrated, and then the sample was purified by silica gel column chromatography to prepare 4.4 g of Compound 1-67. (Yield: 42%, MS: [M+H]⁺=598)

### Synthesis Example 1-68

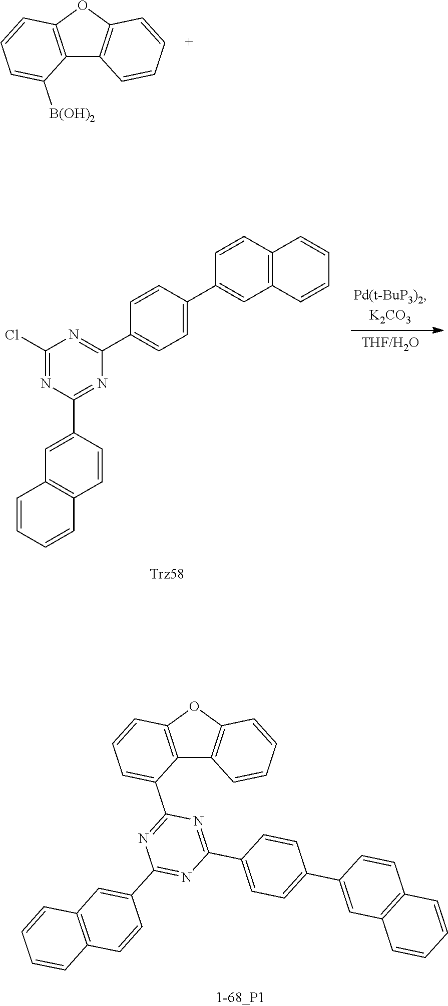 [SMILES: OB(O)c1cccc2oc3ccccc3c12 ; Clc1nc(-c2ccc(-c3ccc4ccccc4c3)cc2)nc(-c2ccc3ccccc3c2)n1 ; c1ccc2cc(-c3ccc(-c4nc(-c5ccc6ccccc6c5)nc(-c5cccc6oc7ccccc7c56)n4)cc3)ccc2c1]

Dibenzo[b,d]furan-1-ylboronic acid (15 g, 70.8 mmol) and Compound Trz58 (33 g, 74.3 mmol) were added to 300 ml of THF, and the mixture was stirred and refluxed. Then, potassium carbonate (29.3 g, 212.3 mmol) was dissolved in 88 ml of water and added thereto, and the mixture was sufficiently stirred and then bis(tri-tert-butylphosphine)palladium(0) (0.4 g, 0.7 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 26.5 g of Compound 1-68_P1. (Yield 65% MS: [M+H]⁺=576).

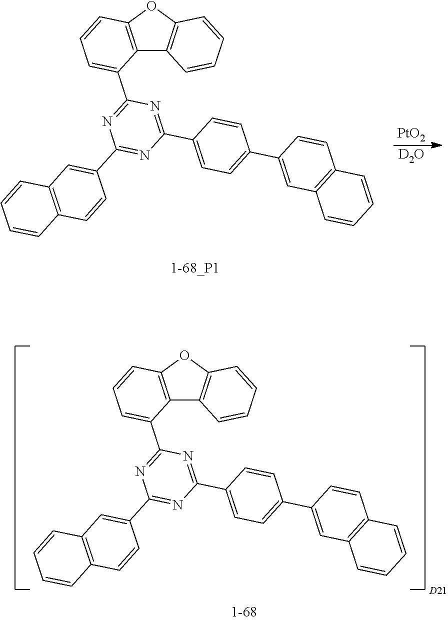

Compound 1-68_P1 (10 g, 17.4 mmol), PtO₂ (1.2 g, 5.2 mmol) and D₂O (87 ml) were added to a shaker tube, and then the tube was sealed and heated at 250° C. and 600 psi for 12 hours. When the reaction was completed, chloroform was added, and the reaction solution was transferred to a separatory funnel, and extracted. The extract was dried over MgSO₄ and concentrated, and then the sample was purified by silica gel column chromatography to prepare 3.6 g of Compound 1-68. (Yield: 35%, MS: [M+H]⁺=598)

### Synthesis Example 2-1

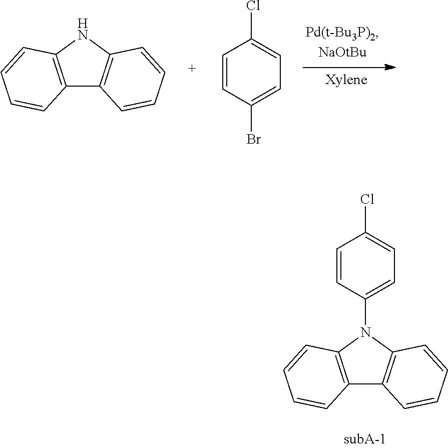 [SMILES: c1ccc2c(c1)[nH]c1ccccc12 ; Clc1ccc(Br)cc1 ; Clc1ccc(-n2c3ccccc3c3ccccc32)cc1]

9H-carbazole (10 g, 59.8 mmol), 1-bromo-4-chlorobenzene (12 g, 62.8 mmol) and sodium tert-butoxide (7.5 g, 77.7 mmol) were added to 200 ml of xylene under a nitrogen atmosphere, and the mixture was stirred and refluxed. Then, bis(tri-tert-butylphosphine)palladium(0)(0.3 g, 0.6 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 10.3 g of Compound subA-1. (Yield: 62%, MS: [M+H]⁺=278)

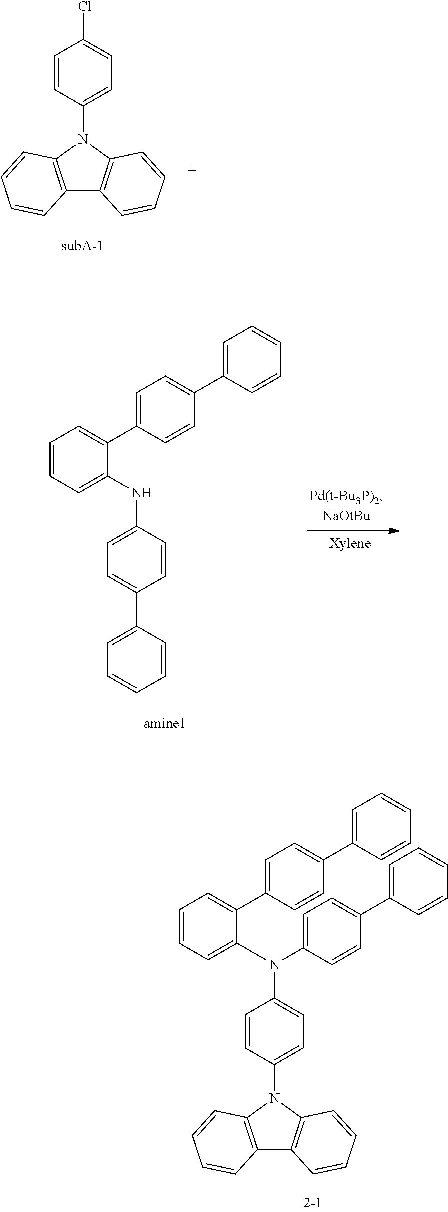 [SMILES: Clc1ccc(-n2c3ccccc3c3ccccc32)cc1 ; c1ccc(-c2ccc(Nc3ccccc3-c3ccc(-c4ccccc4)cc3)cc2)cc1 ; c1ccc(-c2ccc(-c3ccccc3N(c3ccc(-c4ccccc4)cc3)c3ccc(-n4c5ccccc5c5ccccc54)cc3)cc2)cc1]

Compound subA-1 (10 g, 36 mmol), Compound amine1 (15 g, 37.8 mmol) and sodium tert-butoxide (4.5 g, 46.8 mmol) were added to 200 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 14.2 g of Compound 2-1. (Yield: 62%, MS: [M+H]⁺=639)

### Synthesis Example 2-2

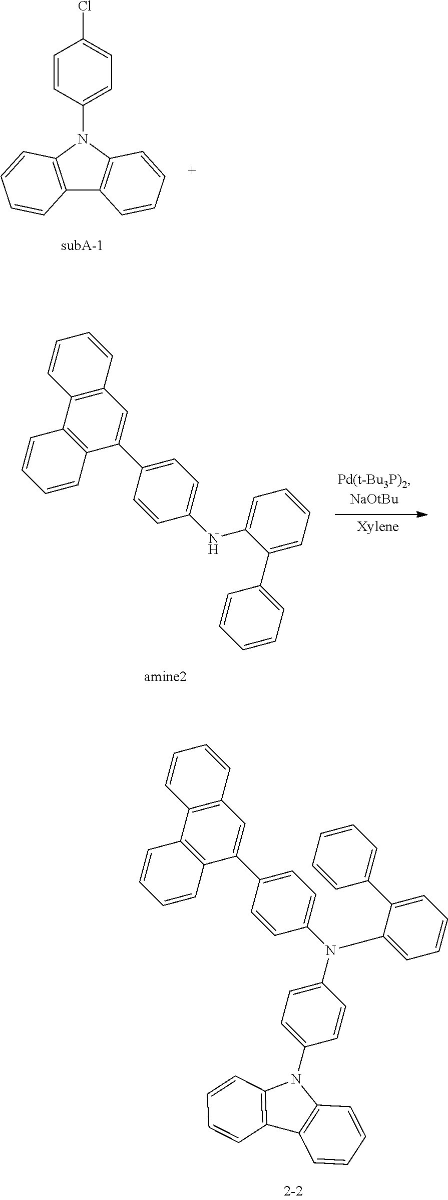 [SMILES: Clc1ccc(-n2c3ccccc3c3ccccc32)cc1 ; c1ccc(-c2ccccc2Nc2ccc(-c3cc4ccccc4c4ccccc34)cc2)cc1 ; c1ccc(-c2ccccc2N(c2ccc(-c3cc4ccccc4c4ccccc34)cc2)c2ccc(-n3c4ccccc4c4ccccc43)cc2)cc1]

Compound subA-1 (10 g, 36 mmol), Compound amine2 (15.9 g, 37.8 mmol) and sodium tert-butoxide (4.5 g, 46.8 mmol) were added to 200 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 17.4 g of Compound 2-2. (Yield: 73%, MS: [M+H]⁺=663) Synthesis Example 2-3

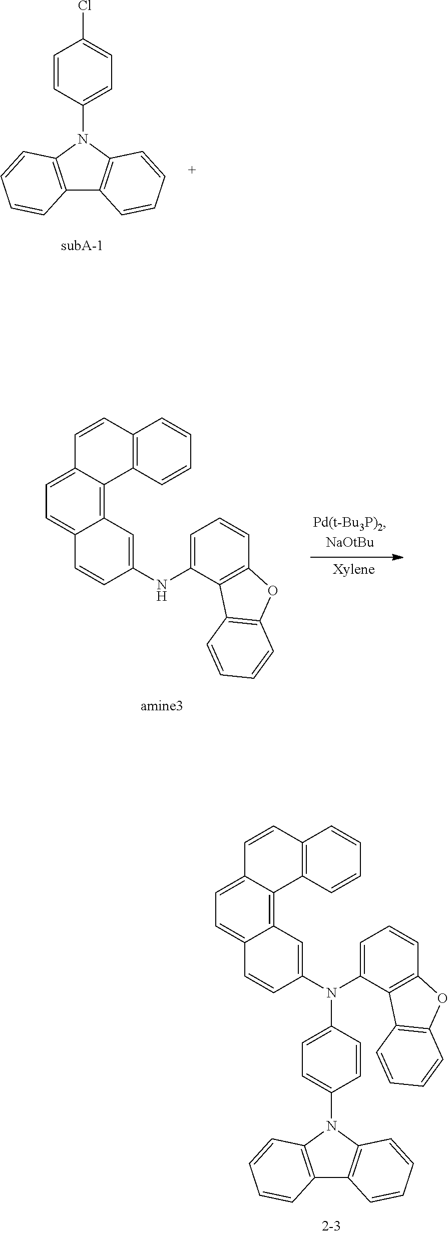 [SMILES: Clc1ccc(-n2c3ccccc3c3ccccc32)cc1 ; c1ccc2c(c1)ccc1ccc3ccc(Nc4cccc5oc6ccccc6c45)cc3c12 ; c1ccc2c(c1)ccc1ccc3ccc(N(c4ccc(-n5c6ccccc6c6ccccc65)cc4)c4cccc5oc6ccccc6c45)cc3c12]

Compound subA-1 (10 g, 36 mmol), Compound amine3 (15.5 g, 37.8 mmol) and sodium tert-butoxide (4.5 g, 46.8 mmol) were added to 200 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 14.3 g of Compound 2-3. (Yield: 61%, MS: [M+H]⁺=651)

### Synthesis Example 2-4

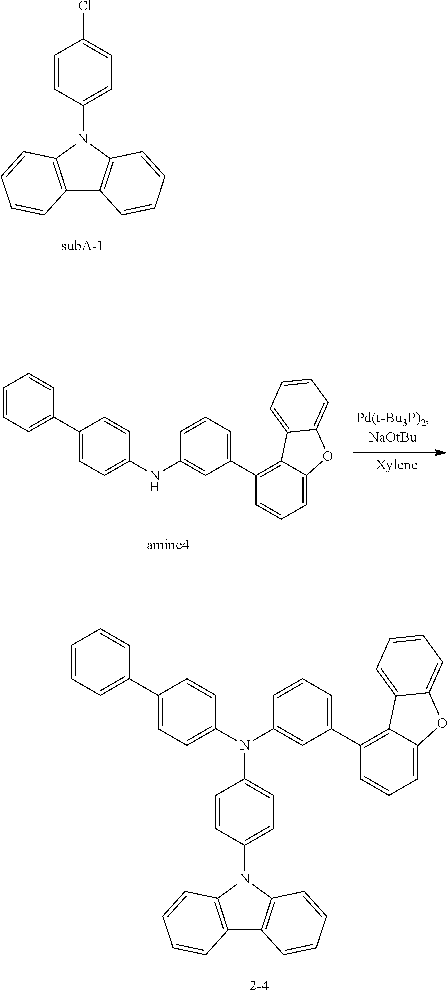 [SMILES: Clc1ccc(-n2c3ccccc3c3ccccc32)cc1 ; c1ccc(-c2ccc(Nc3cccc(-c4cccc5oc6ccccc6c45)c3)cc2)cc1 ; c1ccc(-c2ccc(N(c3ccc(-n4c5ccccc5c5ccccc54)cc3)c3cccc(-c4cccc5oc6ccccc6c45)c3)cc2)cc1]

Compound subA-1 (10 g, 36 mmol), Compound amine4 (15.6 g, 37.8 mmol) and sodium tert-butoxide (4.5 g, 46.8 mmol) were added to 200 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 14.3 g of Compound 2-4. (Yield: 61%, MS: [M+H]⁺=653)

### Synthesis Example 2-5

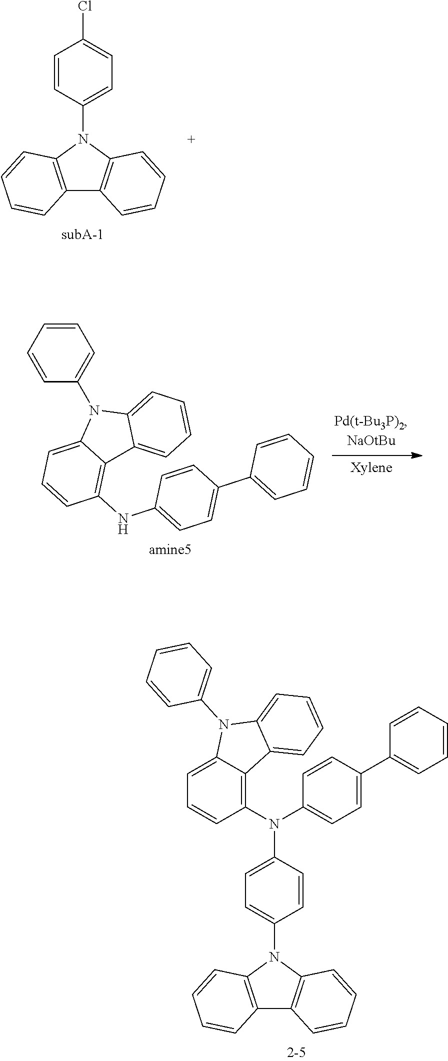 [SMILES: Clc1ccc(-n2c3ccccc3c3ccccc32)cc1 ; c1ccc(-c2ccc(Nc3cccc4c3c3ccccc3n4-c3ccccc3)cc2)cc1 ; c1ccc(-c2ccc(N(c3ccc(-n4c5ccccc5c5ccccc54)cc3)c3cccc4c3c3ccccc3n4-c3ccccc3)cc2)cc1]

Compound subA-1 (10 g, 36 mmol), Compound amine5 (15.5 g, 37.8 mmol) and sodium tert-butoxide (4.5 g, 46.8 mmol) were added to 200 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 15 g of Compound 2-5. (Yield: 64%, MS: [M+H]⁺=652)

### Synthesis Example 2-6

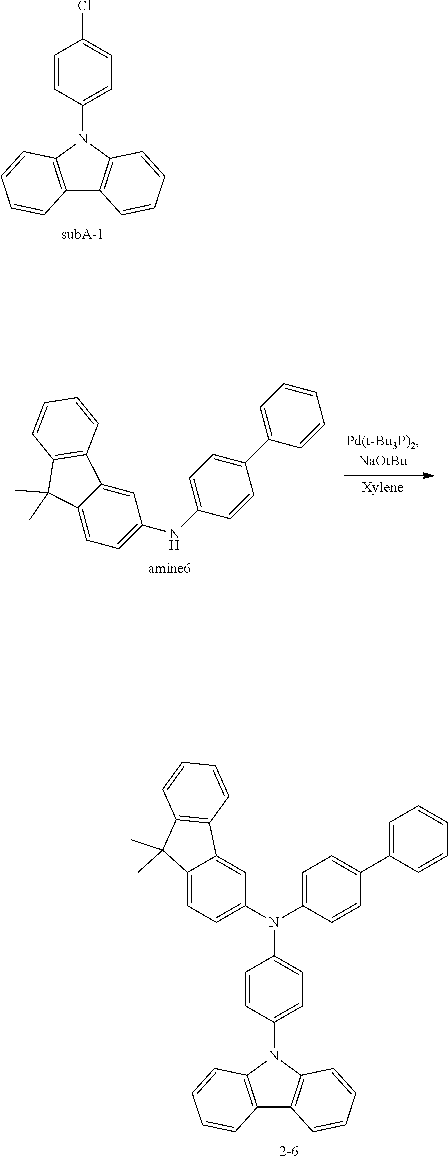 [SMILES: Clc1ccc(-n2c3ccccc3c3ccccc32)cc1 ; CC1(C)c2ccccc2-c2cc(Nc3ccc(-c4ccccc4)cc3)ccc21 ; CC1(C)c2ccccc2-c2cc(N(c3ccc(-c4ccccc4)cc3)c3ccc(-n4c5ccccc5c5ccccc54)cc3)ccc21]

Compound subA-1 (10 g, 36 mmol), Compound amine6 (13.7 g, 37.8 mmol) and sodium tert-butoxide (4.5 g, 46.8 mmol) were added to 200 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 15.8 g of Compound 2-6. (Yield: 73%, MS: [M+H]⁺=603)

### Synthesis Example 2-7

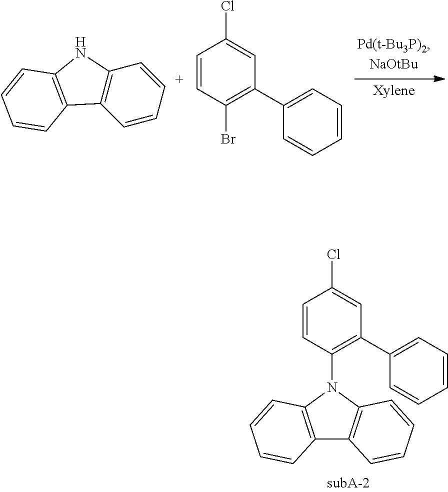 [SMILES: [C+]1=Cc2[nH]c3ccccc3c2C=C1 ; Clc1ccc(Br)c(-c2ccccc2)c1 ; Clc1ccc(-n2c3ccccc3c3ccccc32)c(-c2ccccc2)c1]

9H-carbazole (10 g, 59.8 mmol), 2-bromo-5-chloro-1,1′-bipheny (16.8 g, 62.8 mmol) and sodium tert-butoxide (7.5 g, 77.7 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 14.8 g of Compound subA-2. (Yield: 70%, MS: [M+H]⁺=354)

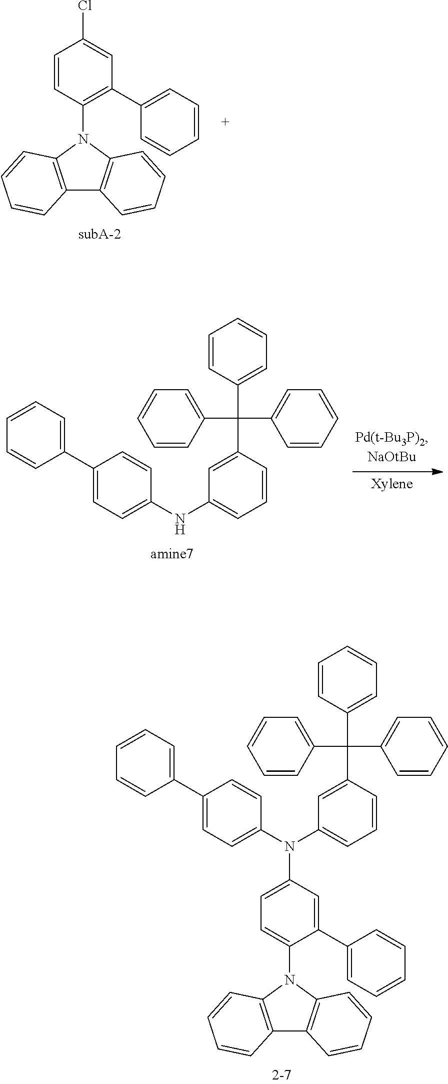 [SMILES: Clc1ccc(-n2c3ccccc3c3ccccc32)c(-c2ccccc2)c1 ; c1ccc(-c2ccc(Nc3cccc(C(c4ccccc4)(c4ccccc4)c4ccccc4)c3)cc2)cc1 ; c1ccc(-c2ccc(N(c3cccc(C(c4ccccc4)(c4ccccc4)c4ccccc4)c3)c3ccc(-n4c5ccccc5c5ccccc54)c(-c4ccccc4)c3)cc2)cc1]

Compound subA-2 (10 g, 28.3 mmol), Compound amine7 (14.5 g, 29.7 mmol) and sodium tert-butoxide (3.5 g, 36.7 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 15.7 g of Compound 2-7. (Yield: 69%, MS: [M+H]⁺=805)

### Synthesis Example 2-8

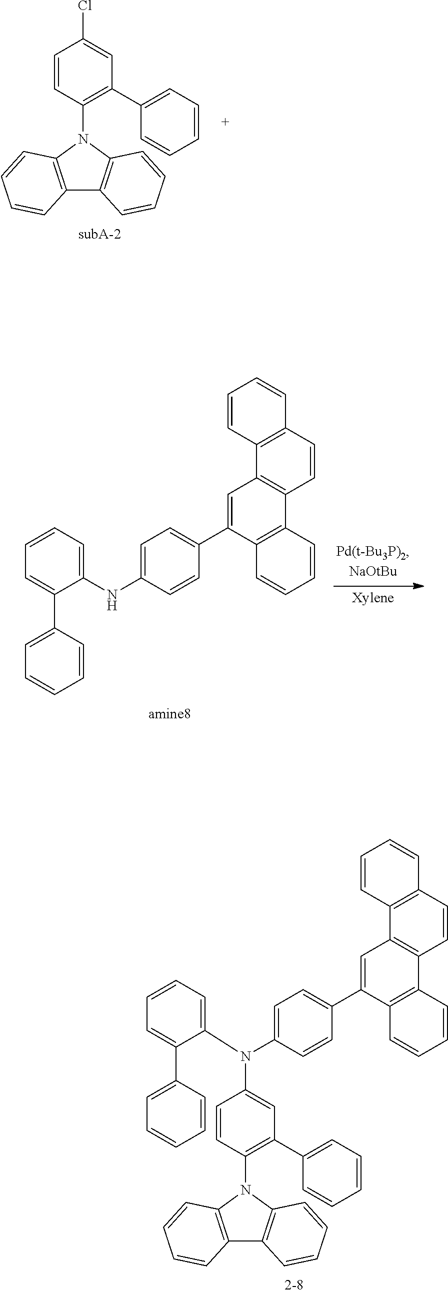 [SMILES: Clc1ccc(-n2c3ccccc3c3ccccc32)c(-c2ccccc2)c1 ; c1ccc(-c2ccccc2Nc2ccc(-c3cc4c5ccccc5ccc4c4ccccc34)cc2)cc1 ; c1ccc(-c2ccccc2N(c2ccc(-c3cc4c5ccccc5ccc4c4ccccc34)cc2)c2ccc(-n3c4ccccc4c4ccccc43)c(-c3ccccc3)c2)cc1]

Compound subA-2 (10 g, 28.3 mmol), Compound amine8 (14 g, 29.7 mmol) and sodium tert-butoxide (3.5 g, 36.7 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.6 g of Compound 2-8. (Yield: 61%, MS: [M+H]⁺=789)

### Synthesis Example 2-9

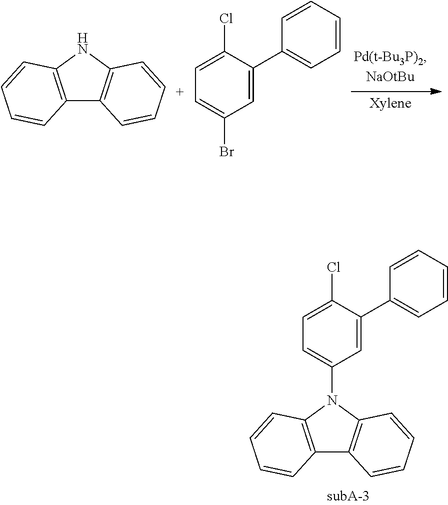 [SMILES: [C+]1=Cc2[nH]c3ccccc3c2C=C1 ; Clc1ccc(Br)cc1-c1ccccc1 ; Clc1ccc(-n2c3ccccc3c3ccccc32)cc1-c1ccccc1]

9H-carbazole (10 g, 59.8 mmol), 5-bromo-2-chloro-1,1′-biphenyl (16.8 g, 62.8 mmol) and sodium tert-butoxide (7.5 g, 77.7 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 15.6 g of Compound subA-3. (Yield: 74%, MS: [M+H]⁺=354)

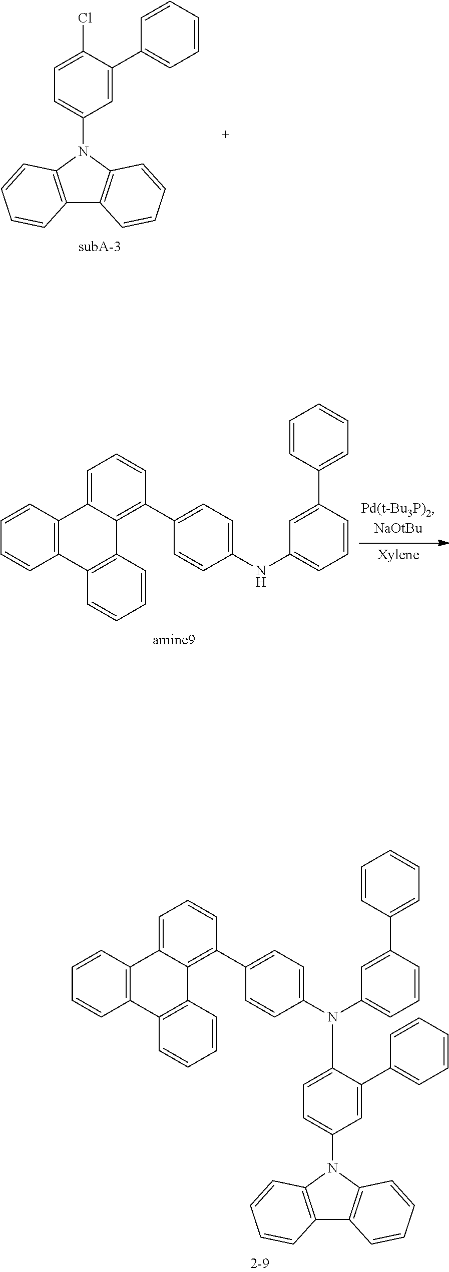 [SMILES: Clc1ccc(-n2c3ccccc3c3ccccc32)cc1-c1ccccc1 ; c1ccc(-c2cccc(Nc3ccc(-c4cccc5c6ccccc6c6ccccc6c45)cc3)c2)cc1 ; c1ccc(-c2cccc(N(c3ccc(-c4cccc5c6ccccc6c6ccccc6c45)cc3)c3ccc(-n4c5ccccc5c5ccccc54)cc3-c3ccccc3)c2)cc1]

Compound subA-3 (10 g, 28.3 mmol), Compound amine9 (14 g, 29.7 mmol) and sodium tert-butoxide (3.5 g, 36.7 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.4 g of Compound 2-9. (Yield: 60%, MS: [M+H]⁺=789)

### Synthesis Example 2-10

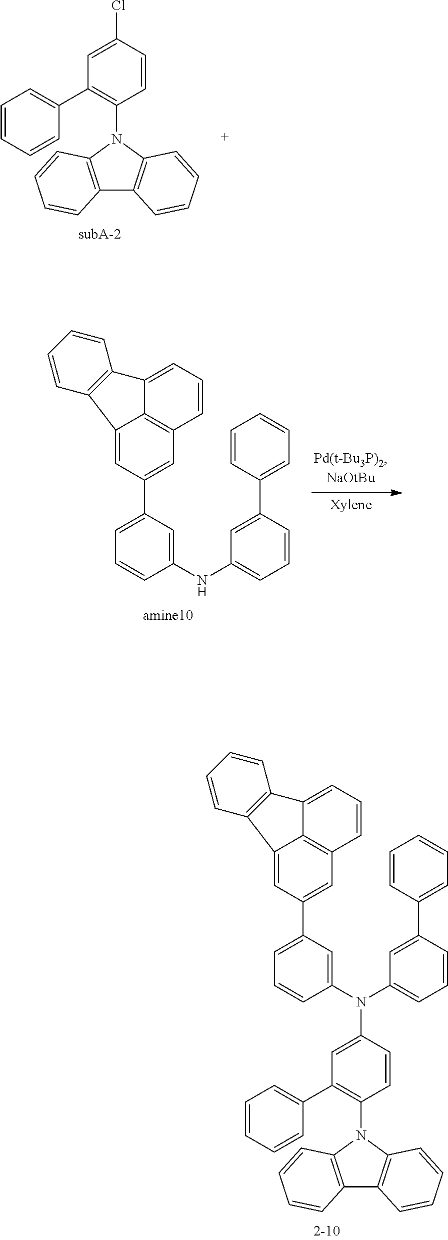 [SMILES: Clc1ccc(-n2c3ccccc3c3ccccc32)c(-c2ccccc2)c1 ; c1ccc(-c2cccc(Nc3cccc(-c4cc5c6c(cccc6c4)-c4ccccc4-5)c3)c2)cc1 ; c1ccc(-c2cccc(N(c3cccc(-c4cc5c6c(cccc6c4)-c4ccccc4-5)c3)c3ccc(-n4c5ccccc5c5ccccc54)c(-c4ccccc4)c3)c2)cc1]

Compound subA-2 (10 g, 28.3 mmol), Compound amine10 (13.2 g, 29.7 mmol) and sodium tert-butoxide (3.5 g, 36.7 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 15.9 g of Compound 2-10. (Yield: 74%, MS: [M+H]⁺=763)

### Synthesis Example 2-11

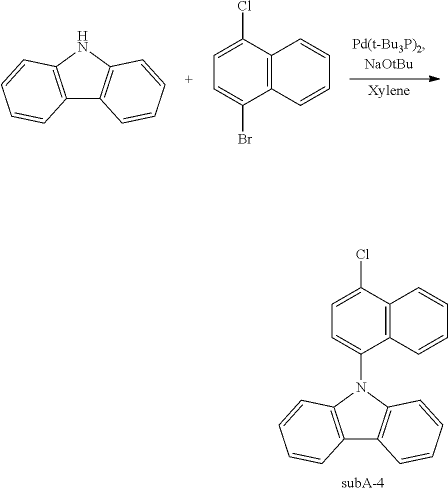 [SMILES: c1ccc2c(c1)[nH]c1ccccc12 ; Clc1ccc(Br)c2ccccc12 ; Clc1ccc(-n2c3ccccc3c3ccccc32)c2ccccc12]

9H-carbazole (10 g, 59.8 mmol), 1-bromo-4-chloronaphthalene (15.2 g, 62.8 mmol) and sodium tert-butoxide (7.5 g, 77.7 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 12.7 g of Compound subA-4. (Yield: 65%, MS: [M+H]⁺=328)

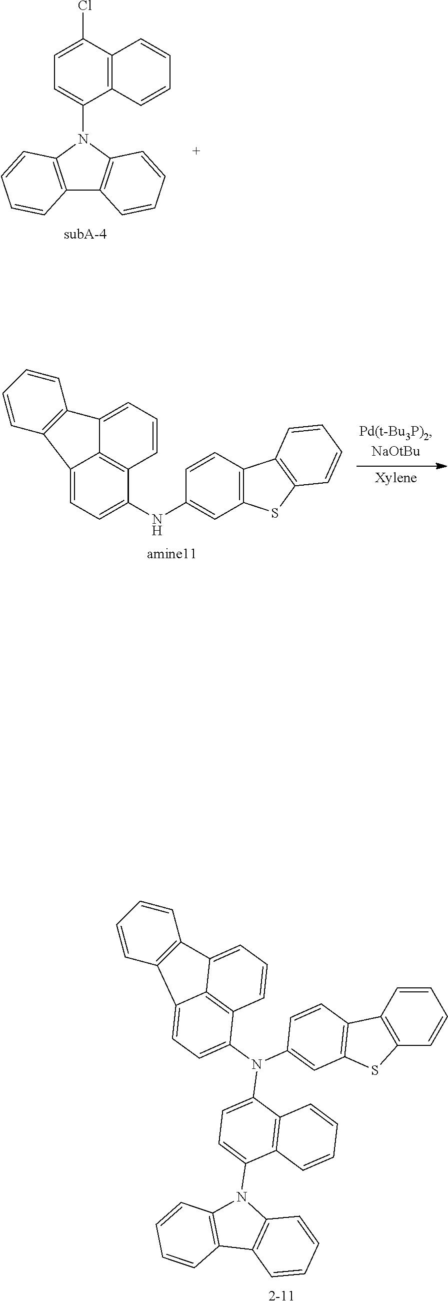 [SMILES: Clc1ccc(-n2c3ccccc3c3ccccc32)c2ccccc12 ; c1ccc2c(c1)-c1cccc3c(Nc4ccc5c(c4)sc4ccccc45)ccc-2c13 ; c1ccc2c(c1)-c1cccc3c(N(c4ccc5c(c4)sc4ccccc45)c4ccc(-n5c6ccccc6c6ccccc65)c5ccccc45)ccc-2c13]

Compound subA-4 (10 g, 30.5 mmol), Compound amine11 (12.8 g, 32 mmol) and sodium tert-butoxide (3.8 g, 39.7 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.9 g of Compound 2-11. (Yield: 66%, MS: [M+H]⁺=691)

### Synthesis Example 2-12

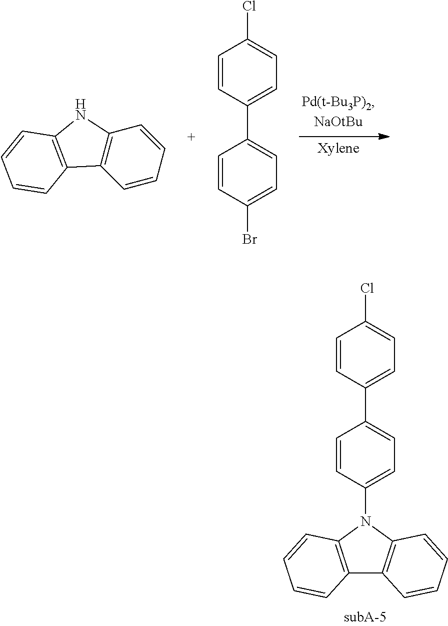 [SMILES: c1ccc2c(c1)[nH]c1ccccc12 ; Clc1ccc(-c2ccc(Br)cc2)cc1 ; Clc1ccc(-c2ccc(-n3c4ccccc4c4ccccc43)cc2)cc1]

9H-carbazole (10 g, 59.8 mmol), 4-bromo-4′-chloro-1,1′-biphenyl (16.8 g, 62.8 mmol) and sodium tert-butoxide (7.5 g, 77.7 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 15.4 g of Compound subA-5. (Yield: 73%, MS: [M+H]⁺=354)

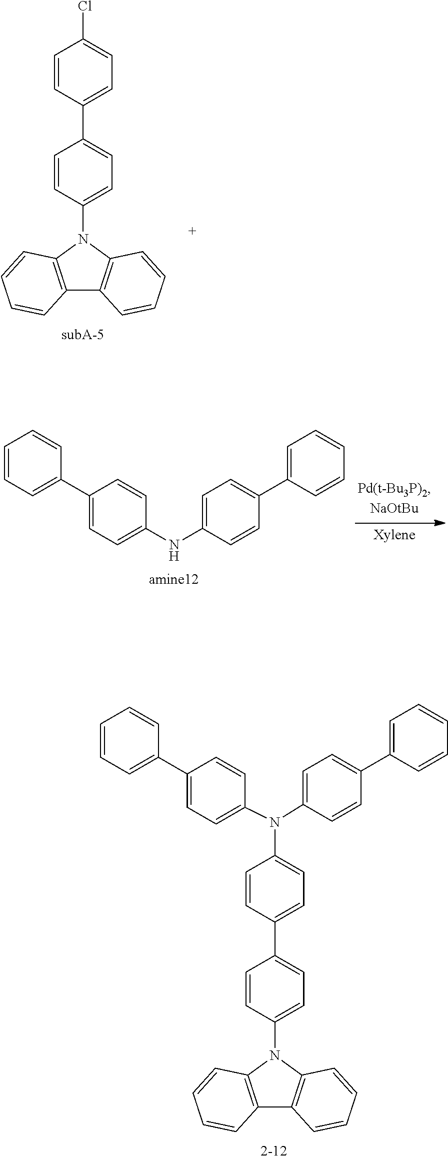 [SMILES: Clc1ccc(-c2ccc(-n3c4ccccc4c4ccccc43)cc2)cc1 ; c1ccc(-c2ccc(Nc3ccc(-c4ccccc4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1]

Compound subA-5 (10 g, 28.3 mmol), Compound amine12 (9.5 g, 29.7 mmol) and sodium tert-butoxide (3.5 g, 36.7 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 11.2 g of Compound 2-12. (Yield: 62%, MS: [M+H]⁺=639)

### Synthesis Example 2-13

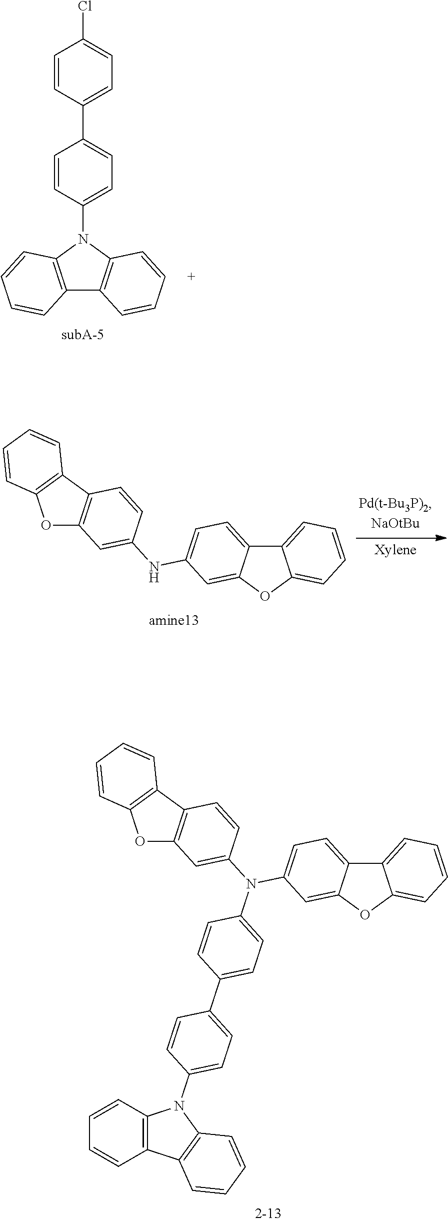 [SMILES: Clc1ccc(-c2ccc(-n3c4ccccc4c4ccccc43)cc2)cc1 ; c1ccc2c(c1)oc1cc(Nc3ccc4c(c3)oc3ccccc34)ccc12 ; c1ccc2c(c1)oc1cc(N(c3ccc(-c4ccc(-n5c6ccccc6c6ccccc65)cc4)cc3)c3ccc4c(c3)oc3ccccc34)ccc12]

Compound subA-5 (10 g, 28.3 mmol), Compound amine13 (10.4 g, 29.7 mmol) and sodium tert-butoxide (3.5 g, 36.7 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.5 g of Compound 2-13. (Yield: 72%, MS: [M+H]⁺=664)

### Synthesis Example 2-14

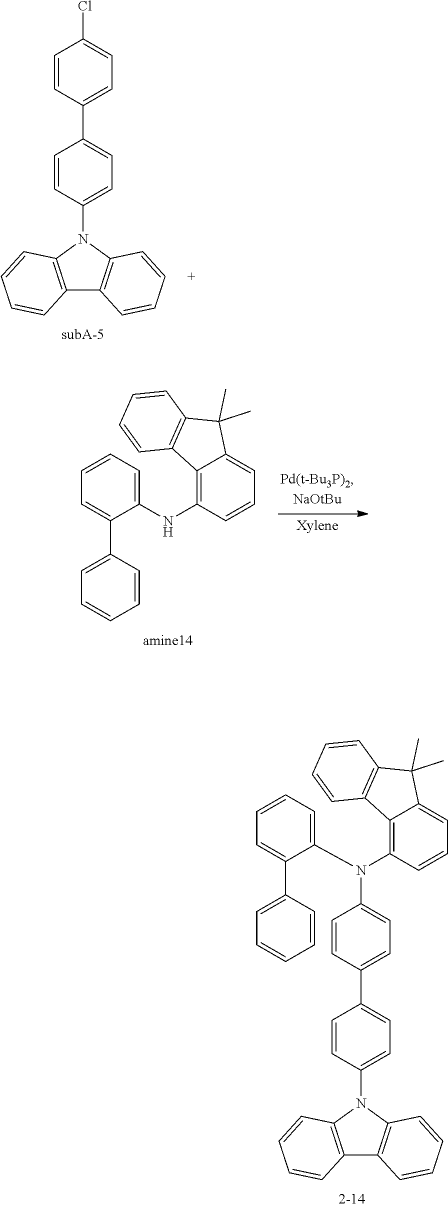 [SMILES: Clc1ccc(-c2ccc(-n3c4ccccc4c4ccccc43)cc2)cc1 ; CC1(C)c2ccccc2-c2c(Nc3ccccc3-c3ccccc3)cccc21 ; CC1(C)c2ccccc2-c2c(N(c3ccc(-c4ccc(-n5c6ccccc6c6ccccc65)cc4)cc3)c3ccccc3-c3ccccc3)cccc21]

Compound subA-5 (10 g, 28.3 mmol), Compound amine14 (10.7 g, 29.7 mmol) and sodium tert-butoxide (3.5 g, 36.7 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 12.5 g of Compound 2-14. (Yield: 65%, MS: [M+H]⁺=679)

### Synthesis Example 2-15

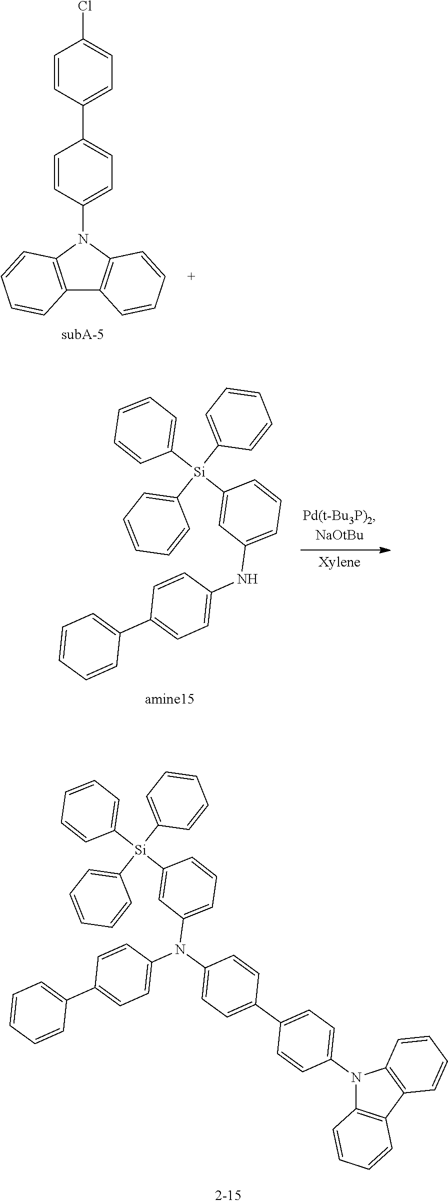 [SMILES: Clc1ccc(-c2ccc(-n3c4ccccc4c4ccccc43)cc2)cc1 ; c1ccc(-c2ccc(Nc3cccc([Si](c4ccccc4)(c4ccccc4)c4ccccc4)c3)cc2)cc1 ; c1ccc(-c2ccc(N(c3ccc(-c4ccc(-n5c6ccccc6c6ccccc65)cc4)cc3)c3cccc([Si](c4ccccc4)(c4ccccc4)c4ccccc4)c3)cc2)cc1]

Compound subA-5 (10 g, 28.3 mmol), Compound amine15 (14.9 g, 29.7 mmol) and sodium tert-butoxide (3.5 g, 36.7 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 14.6 g of Compound 2-15. (Yield: 63%, MS: [M+H]⁺=821)

### Synthesis Example 2-16

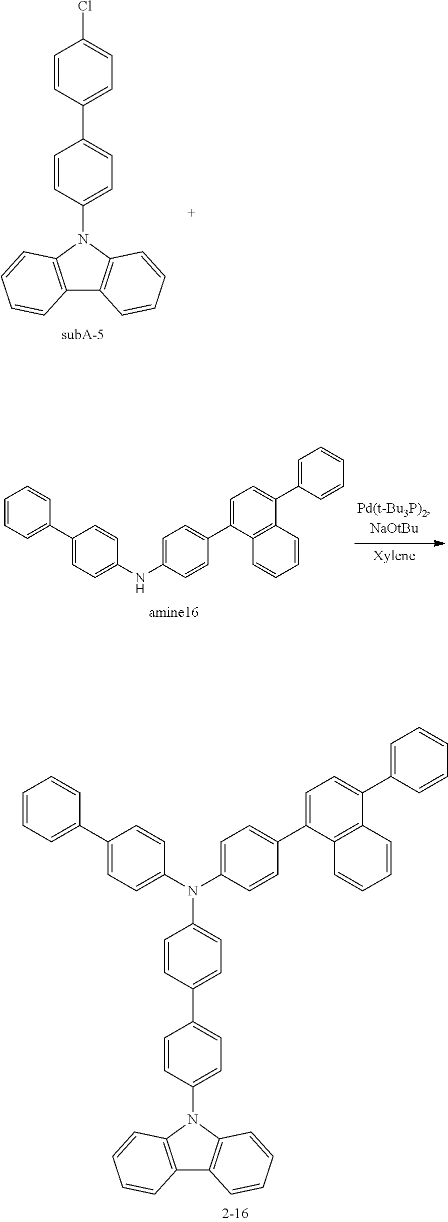 [SMILES: Clc1ccc(-c2ccc(-n3c4ccccc4c4ccccc43)cc2)cc1 ; c1ccc(-c2ccc(Nc3ccc(-c4ccc(-c5ccccc5)c5ccccc45)cc3)cc2)cc1 ; c1ccc(-c2ccc(N(c3ccc(-c4ccc(-n5c6ccccc6c6ccccc65)cc4)cc3)c3ccc(-c4ccc(-c5ccccc5)c5ccccc45)cc3)cc2)cc1]

Compound subA-5 (10 g, 28.3 mmol), Compound amine16 (13.3 g, 29.7 mmol) and sodium tert-butoxide (3.5 g, 36.7 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 15.6 g of Compound 2-16. (Yield: 72%, MS: [M+H]⁺=765)

### Synthesis Example 2-17

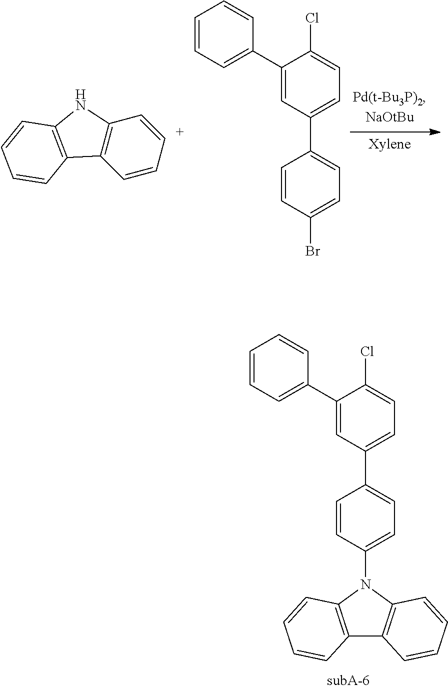 [SMILES: [C+]1=Cc2[nH]c3ccccc3c2C=C1 ; Clc1ccc(-c2ccc(Br)cc2)cc1-c1ccccc1 ; Clc1ccc(-c2ccc(-n3c4ccccc4c4ccccc43)cc2)cc1-c1ccccc1]

9H-carbazole (10 g, 59.8 mmol), 4-bromo-4′-chloro-1,1′:3′,1″-terphenyl (21.6 g, 62.8 mmol) and sodium tert-butoxide (7.5 g, 77.7 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 15.4 g of Compound subA-6. (Yield: 60%, MS: [M+H]⁺=430)

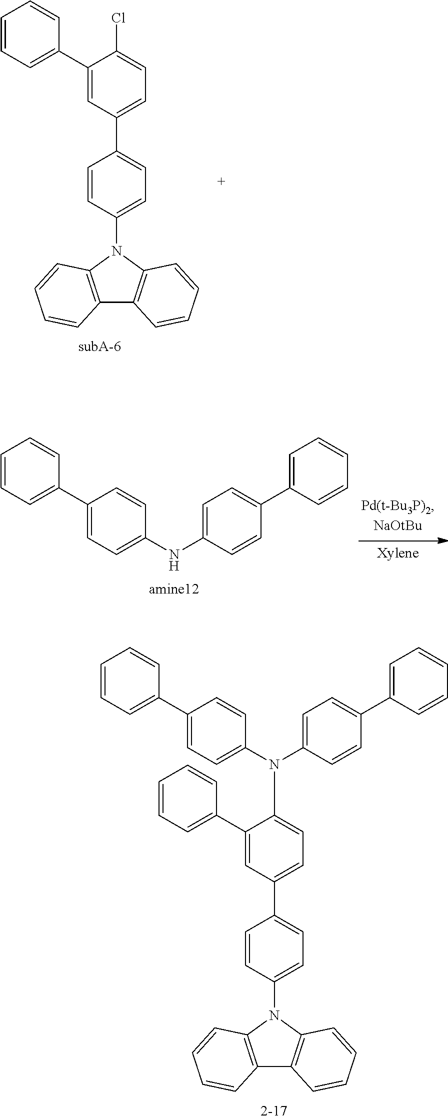 [SMILES: Clc1ccc(-c2ccc(-n3c4ccccc4c4ccccc43)cc2)cc1-c1ccccc1 ; c1ccc(-c2ccc(Nc3ccc(-c4ccccc4)cc3)cc2)cc1 ; c1ccc(-c2ccc(N(c3ccc(-c4ccccc4)cc3)c3ccc(-c4ccc(-n5c6ccccc6c6ccccc65)cc4)cc3-c3ccccc3)cc2)cc1]

Compound subA-6 (10 g, 23.3 mmol), Compound amine12 (7.8 g, 24.4 mmol) and sodium tert-butoxide (2.9 g, 30.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 11.5 g of Compound 2-17. (Yield: 69%, MS: [M+H]⁺=715)

### Synthesis Example 2-18

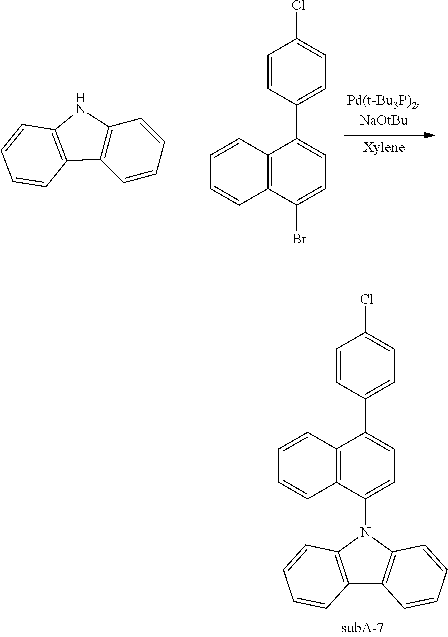 [SMILES: c1ccc2c(c1)[nH]c1ccccc12 ; Clc1ccc(-c2ccc(Br)c3ccccc23)cc1 ; Clc1ccc(-c2ccc(-n3c4ccccc4c4ccccc43)c3ccccc23)cc1]

9H-carbazole (10 g, 59.8 mmol), 1-bromo-4-(4-chlorophenyl) naphthalene (19.9 g, 62.8 mmol) and sodium tert-butoxide (7.5 g, 77.7 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 15.7 g of Compound subA-7. (Yield: 65%, MS: [M+H]⁺=404)

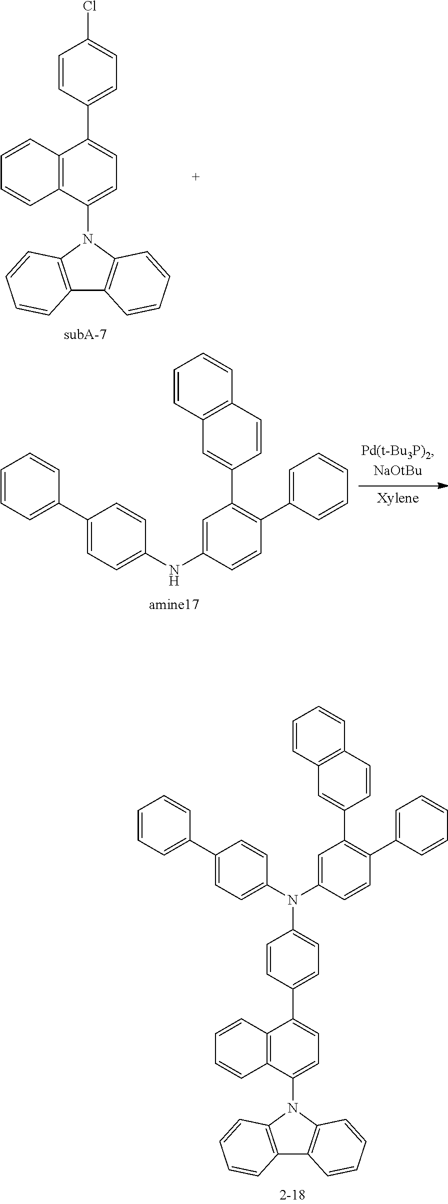 [SMILES: Clc1ccc(-c2ccc(-n3c4ccccc4c4ccccc43)c3ccccc23)cc1 ; c1ccc(-c2ccc(Nc3ccc(-c4ccccc4)c(-c4ccc5ccccc5c4)c3)cc2)cc1 ; c1ccc(-c2ccc(N(c3ccc(-c4ccc(-n5c6ccccc6c6ccccc65)c5ccccc45)cc3)c3ccc(-c4ccccc4)c(-c4ccc5ccccc5c4)c3)cc2)cc1]

Compound subA-7 (10 g, 24.8 mmol), Compound amine17 (11.6 g, 26 mmol) and sodium tert-butoxide (3.1 g, 32.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.1 g of Compound 2-18. (Yield: 65%, MS: [M+H]⁺=815)

### Synthesis Example 2-19

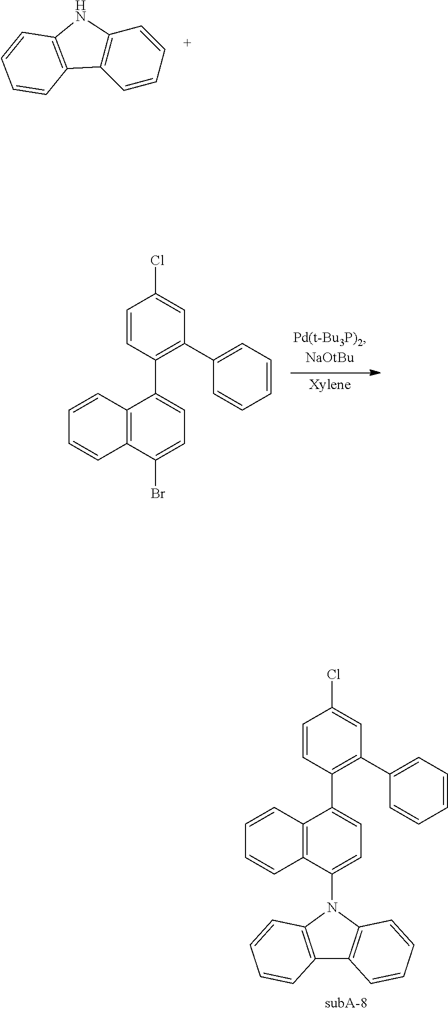 [SMILES: c1ccc2c(c1)[nH]c1ccccc12 ; Clc1ccc(-c2ccc(Br)c3ccccc23)c(-c2ccccc2)c1 ; Clc1ccc(-c2ccc(-n3c4ccccc4c4ccccc43)c3ccccc23)c(-c2ccccc2)c1]

9H-carbazole (10 g, 59.8 mmol), 1-bromo-4-(5-chloro-[1,1′-biphenyl]-2-yl) naphthalene (24.7 g, 62.8 mmol) and sodium tert-butoxide (7.5 g, 77.7 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 18.6 g of Compound subA-8. (Yield: 65%, MS: [M+H]⁺=480)

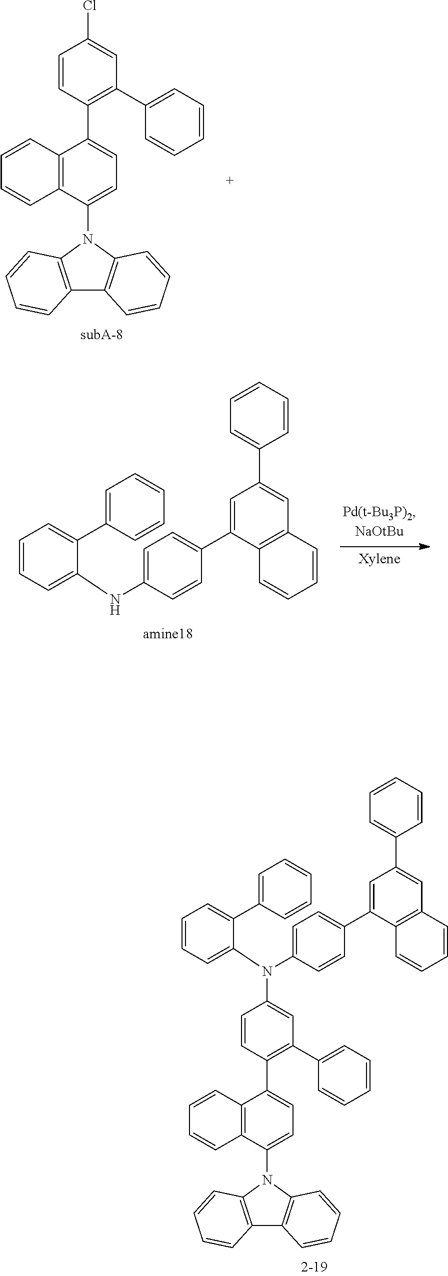 [SMILES: Clc1ccc(-c2ccc(-n3c4ccccc4c4ccccc43)c3ccccc23)c(-c2ccccc2)c1 ; c1ccc(-c2cc(-c3ccc(Nc4ccccc4-c4ccccc4)cc3)c3ccccc3c2)cc1 ; c1ccc(-c2cc(-c3ccc(N(c4ccc(-c5ccc(-n6c7ccccc7c7ccccc76)c6ccccc56)c(-c5ccccc5)c4)c4ccccc4-c4ccccc4)cc3)c3ccccc3c2)cc1]

Compound subA-8 (10 g, 20.8 mmol), Compound amine18 (9.8 g, 21.9 mmol) and sodium tert-butoxide (2.6 g, 27.1 mmol) were added to 200 ml of xylene under a nitrogen atmosphere, and the mixture was stirred and refluxed. Then, bis(tri-tert-butyl phosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.5 g of Compound 2-19. (Yield: 73%, MS: [M+H]⁺=891)

### Synthesis Example 2-20

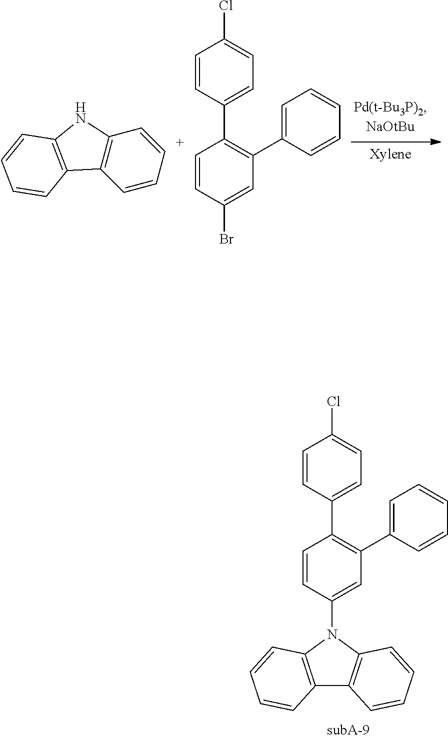

9H-carbazole (10 g, 59.8 mmol), 4′-bromo-4-chloro-1,1′:2′,1″-terphenyl (21.6 g, 62.8 mmol) and sodium tert-butoxide (7.5 g, 77.7 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 15.7 g of Compound subA-9. (Yield: 61%, MS: [M+H]⁺=430)

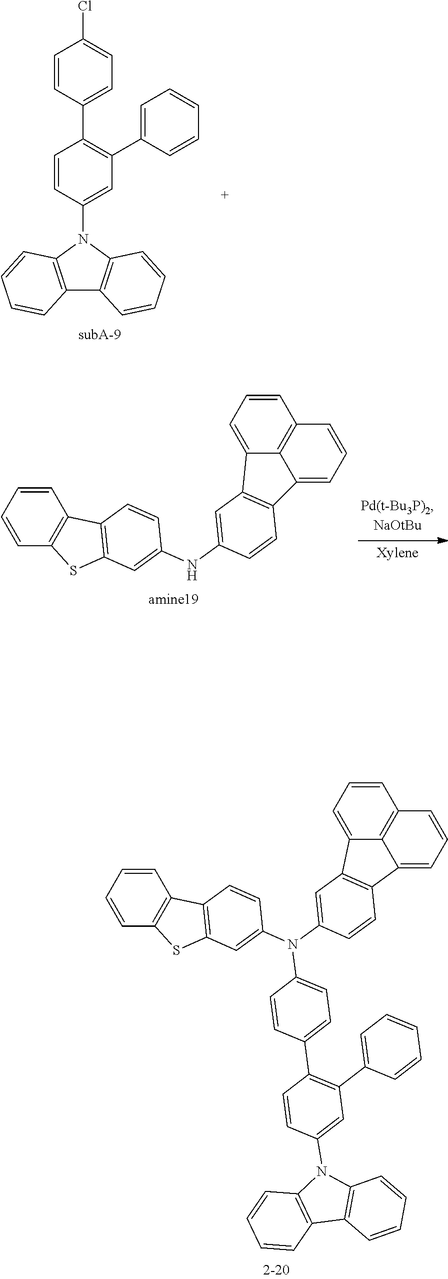 [SMILES: Clc1ccc(-c2ccc(-n3c4ccccc4c4ccccc43)cc2-c2ccccc2)cc1 ; c1cc2c3c(cccc3c1)-c1cc(Nc3ccc4c(c3)sc3ccccc34)ccc1-2 ; c1ccc(-c2cc(-n3c4ccccc4c4ccccc43)ccc2-c2ccc(N(c3ccc4c(c3)-c3cccc5cccc-4c35)c3ccc4c(c3)sc3ccccc34)cc2)cc1]

Compound subA-9 (10 g, 23.3 mmol), Compound amine19 (9.8 g, 24.4 mmol) and sodium tert-butoxide (2.9 g, 30.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 11.6 g of Compound 2-20. (Yield: 63%, MS: [M+H]⁺=793)

### Synthesis Example 2-21

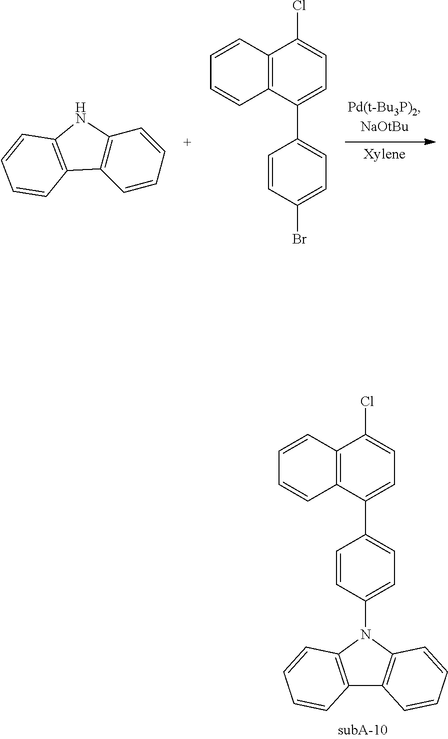 [SMILES: c1ccc2c(c1)[nH]c1ccccc12 ; Clc1ccc(-c2ccc(Br)cc2)c2ccccc12 ; Clc1ccc(-c2ccc(-n3c4ccccc4c4ccccc43)cc2)c2ccccc12]

9H-carbazole (10 g, 59.8 mmol), 1-(4-bromophenyl)-4-chloronaphthalene (19.9 g, 62.8 mmol) and sodium tert-butoxide (7.5 g, 77.7 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 17.4 g of Compound subA-10. (Yield: 72%, MS: [M+H]⁺=404)

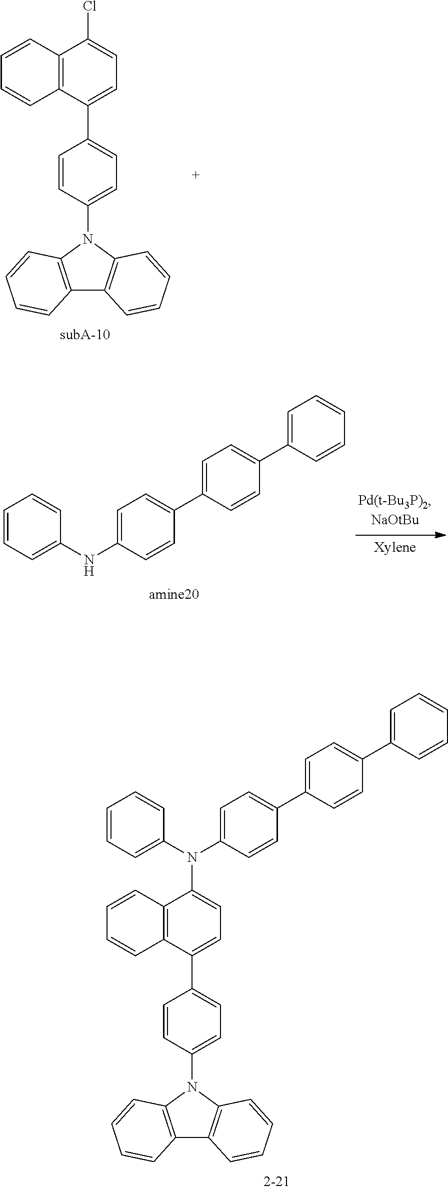 [SMILES: Clc1ccc(-c2ccc(-n3c4ccccc4c4ccccc43)cc2)c2ccccc12 ; c1ccc(Nc2ccc(-c3ccc(-c4ccccc4)cc3)cc2)cc1 ; c1ccc(-c2ccc(-c3ccc(N(c4ccccc4)c4ccc(-c5ccc(-n6c7ccccc7c7ccccc76)cc5)c5ccccc45)cc3)cc2)cc1]

Compound subA-10 (10 g, 24.8 mmol), Compound amine20 (8.4 g, 26 mmol) and sodium tert-butoxide (3.1 g, 32.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 12.6 g of Compound 2-21. (Yield: 74%, MS: [M+H]⁺=689)

### Synthesis Example 2-22

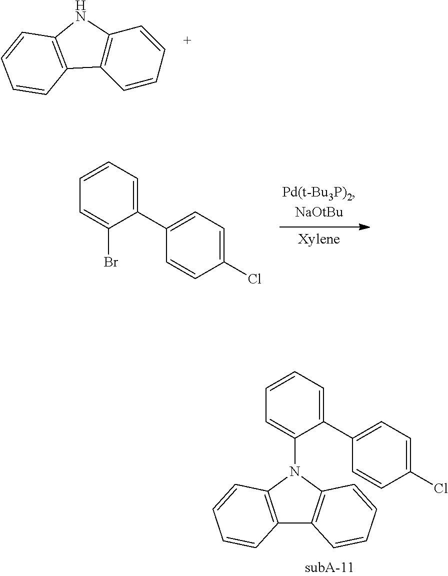 [SMILES: c1ccc2c(c1)[nH]c1ccccc12 ; Clc1ccc(-c2ccccc2Br)cc1 ; Clc1ccc(-c2ccccc2-n2c3ccccc3c3ccccc32)cc1]

9H-carbazole (10 g, 59.8 mmol), 2-bromo-4′-chloro-1,1′-biphenyl (16.8 g, 62.8 mmol) and sodium tert-butoxide (7.5 g, 77.7 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 15.6 g of Compound subA-11. (Yield: 74%, MS: [M+H]⁺=354)

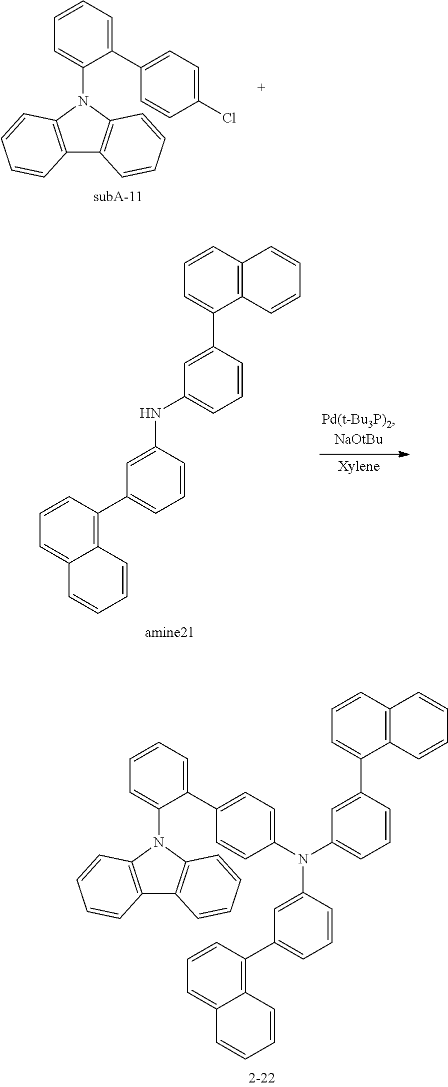 [SMILES: Clc1ccc(-c2ccccc2-n2c3ccccc3c3ccccc32)cc1 ; c1cc(Nc2cccc(-c3cccc4ccccc34)c2)cc(-c2cccc3ccccc23)c1 ; c1cc(-c2cccc3ccccc23)cc(N(c2ccc(-c3ccccc3-n3c4ccccc4c4ccccc43)cc2)c2cccc(-c3cccc4ccccc34)c2)c1]

Compound subA-11 (10 g, 28.3 mmol), Compound amine21 (12.5 g, 29.7 mmol) and sodium tert-butoxide (3.5 g, 36.7 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 12.9 g of Compound 2-22. (Yield: 62%, MS: [M+H]⁺=739)

### Synthesis Example 2-23

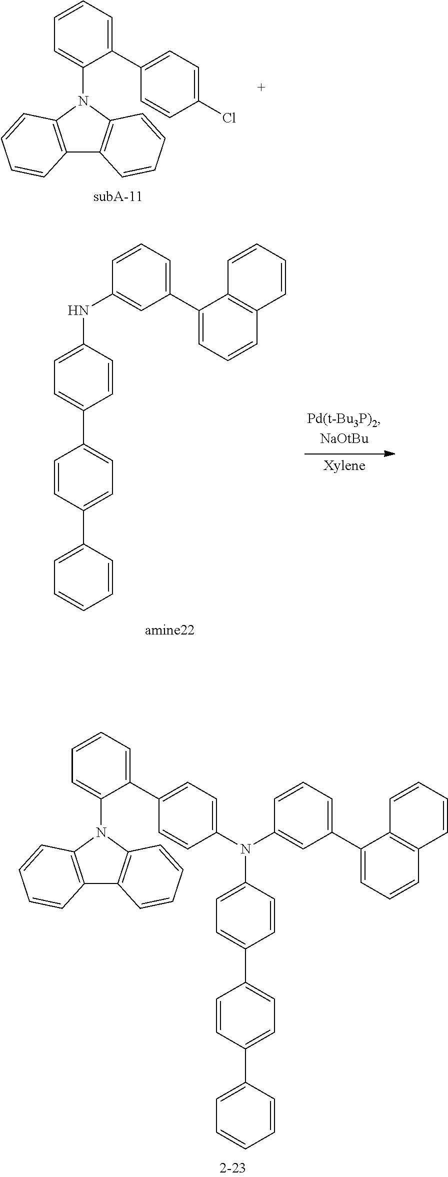 [SMILES: Clc1ccc(-c2ccccc2-n2c3ccccc3c3ccccc32)cc1 ; c1ccc(-c2ccc(-c3ccc(Nc4cccc(-c5cccc6ccccc56)c4)cc3)cc2)cc1 ; c1ccc(-c2ccc(-c3ccc(N(c4ccc(-c5ccccc5-n5c6ccccc6c6ccccc65)cc4)c4cccc(-c5cccc6ccccc56)c4)cc3)cc2)cc1]

Compound subA-11 (10 g, 28.3 mmol), Compound amine22 (13.3 g, 29.7 mmol) and sodium tert-butoxide (3.5 g, 36.7 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.6 g of Compound 2-23. (Yield: 63%, MS: [M+H]⁺=765)

### Synthesis Example 2-24

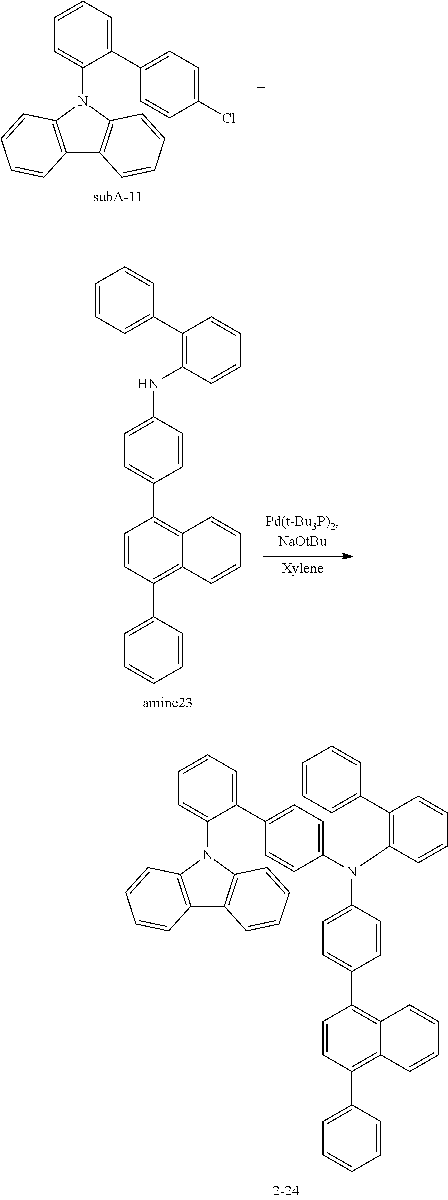 [SMILES: Clc1ccc(-c2ccccc2-n2c3ccccc3c3ccccc32)cc1 ; c1ccc(-c2ccccc2Nc2ccc(-c3ccc(-c4ccccc4)c4ccccc34)cc2)cc1 ; c1ccc(-c2ccccc2N(c2ccc(-c3ccccc3-n3c4ccccc4c4ccccc43)cc2)c2ccc(-c3ccc(-c4ccccc4)c4ccccc34)cc2)cc1]

Compound subA-11 (10 g, 28.3 mmol), Compound amine23 (13.3 g, 29.7 mmol) and sodium tert-butoxide (3.5 g, 36.7 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.4 g of Compound 2-24. (Yield: 62%, MS: [M+H]⁺=765)

### Synthesis Example 2-25

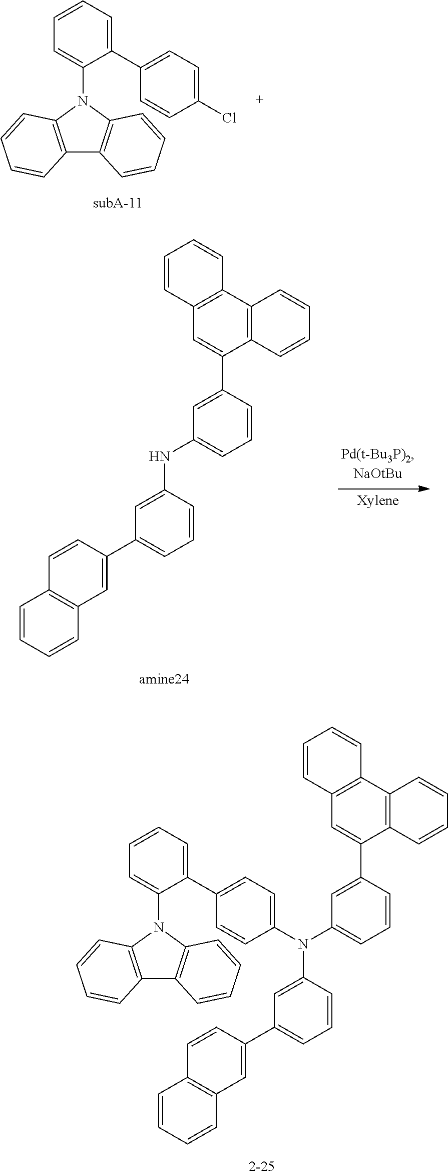 [SMILES: Clc1ccc(-c2ccccc2-n2c3ccccc3c3ccccc32)cc1 ; c1cc(Nc2cccc(-c3cc4ccccc4c4ccccc34)c2)cc(-c2ccc3ccccc3c2)c1 ; c1cc(-c2ccc3ccccc3c2)cc(N(c2ccc(-c3ccccc3-n3c4ccccc4c4ccccc43)cc2)c2cccc(-c3cc4ccccc4c4ccccc34)c2)c1]

Compound subA-11 (10 g, 28.3 mmol), Compound amine24 (14 g, 29.7 mmol) and sodium tert-butoxide (3.5 g, 36.7 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 15.6 g of Compound 2-25. (Yield: 70%, MS: [M+H]⁺=789)

### Synthesis Example 2-26

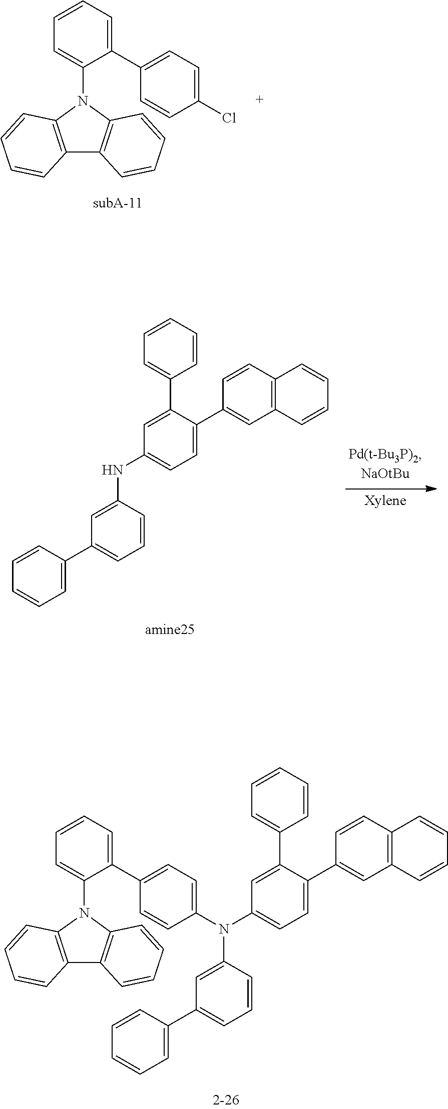 [SMILES: Clc1ccc(-c2ccccc2-n2c3ccccc3c3ccccc32)cc1 ; c1ccc(-c2cccc(Nc3ccc(-c4ccc5ccccc5c4)c(-c4ccccc4)c3)c2)cc1 ; c1ccc(-c2cccc(N(c3ccc(-c4ccccc4-n4c5ccccc5c5ccccc54)cc3)c3ccc(-c4ccc5ccccc5c4)c(-c4ccccc4)c3)c2)cc1]

Compound subA-11 (10 g, 28.3 mmol), Compound amine25 (13.3 g, 29.7 mmol) and sodium tert-butoxide (3.5 g, 36.7 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 14.5 g of Compound 2-26. (Yield: 67%, MS: [M+H]⁺=765)

### Synthesis Example 2-27

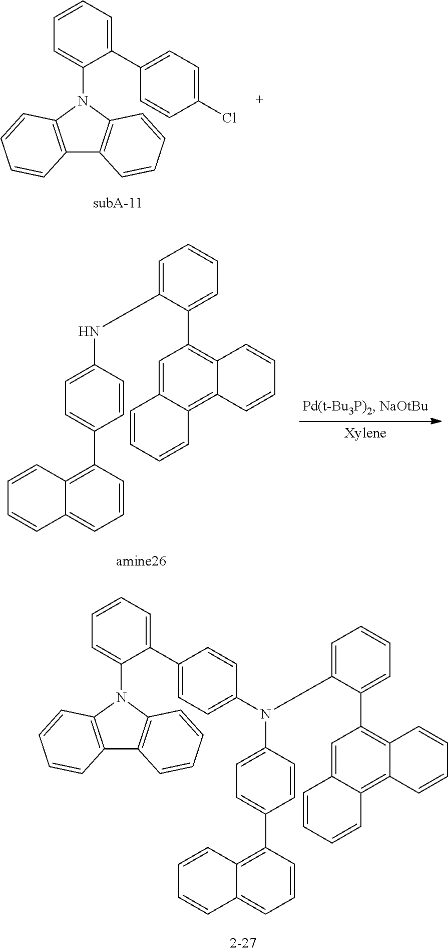 [SMILES: Clc1ccc(-c2ccccc2-n2c3ccccc3c3ccccc32)cc1 ; c1ccc(-c2cc3ccccc3c3ccccc23)c(Nc2ccc(-c3cccc4ccccc34)cc2)c1 ; c1ccc(N(c2ccc(-c3ccccc3-n3c4ccccc4c4ccccc43)cc2)c2ccc(-c3cccc4ccccc34)cc2)c(-c2cc3ccccc3c3ccccc23)c1]

Compound subA-11 (10 g, 28.3 mmol), Compound amine26 (14 g, 29.7 mmol) and sodium tert-butoxide (3.5 g, 36.7 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 16.5 g of Compound 2-27. (Yield: 74%, MS: [M+H]⁺=789)

### Synthesis Example 2-28

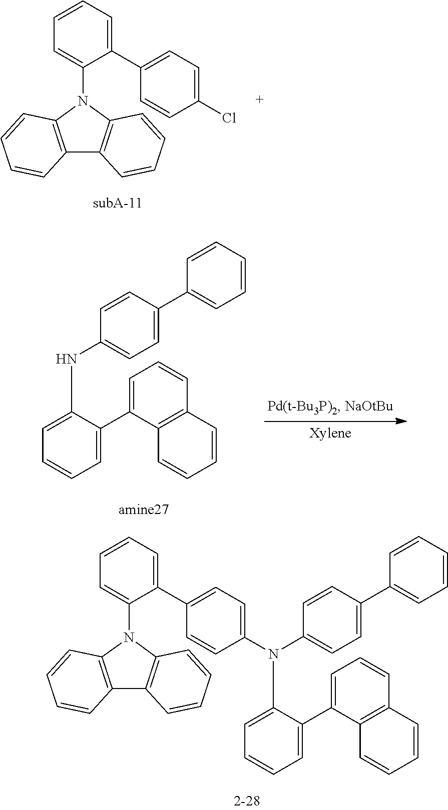 [SMILES: Clc1ccc(-c2ccccc2-n2c3ccccc3c3ccccc32)cc1 ; c1ccc(-c2ccc(Nc3ccccc3-c3cccc4ccccc34)cc2)cc1 ; c1ccc(-c2ccc(N(c3ccc(-c4ccccc4-n4c5ccccc5c5ccccc54)cc3)c3ccccc3-c3cccc4ccccc34)cc2)cc1]

Compound subA-11 (10 g, 28.3 mmol), Compound amine27 (11 g, 29.7 mmol) and sodium tert-butoxide (3.5 g, 36.7 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 11.7 g of Compound 2-28. (Yield: 60%, MS: [M+H]⁺=689)

### Synthesis Example 2-29

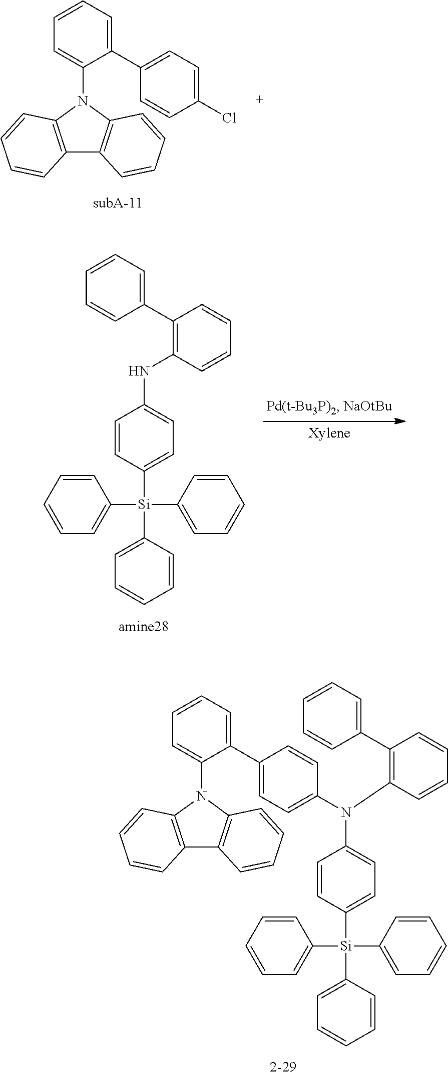 [SMILES: Clc1ccc(-c2ccccc2-n2c3ccccc3c3ccccc32)cc1 ; c1ccc(-c2ccccc2Nc2ccc([Si](c3ccccc3)(c3ccccc3)c3ccccc3)cc2)cc1 ; c1ccc(-c2ccccc2N(c2ccc(-c3ccccc3-n3c4ccccc4c4ccccc43)cc2)c2ccc([Si](c3ccccc3)(c3ccccc3)c3ccccc3)cc2)cc1]

Compound subA-11 (10 g, 28.3 mmol), Compound amine28 (14.9 g, 29.7 mmol) and sodium tert-butoxide (3.5 g, 36.7 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 14.1 g of Compound 2-29. (Yield: 61%, MS: [M+H]⁺=821)

### Synthesis Example 2-30

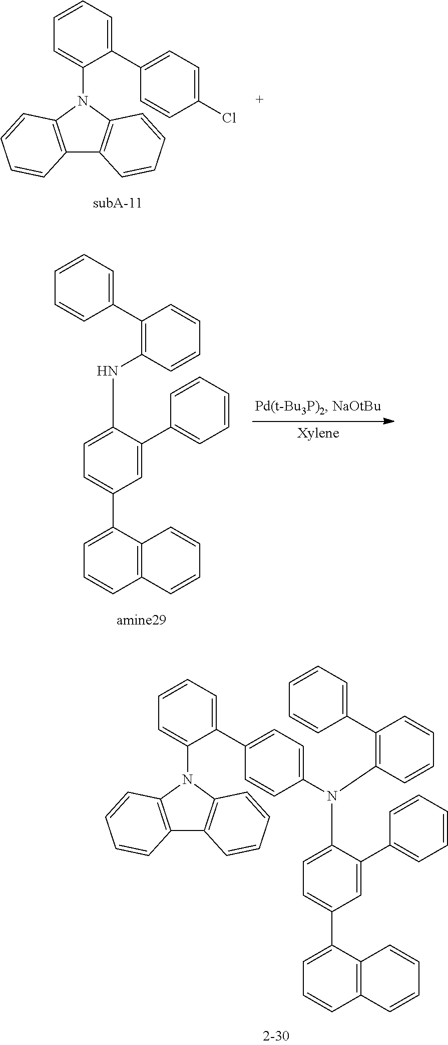 [SMILES: Clc1ccc(-c2ccccc2-n2c3ccccc3c3ccccc32)cc1 ; c1ccc(-c2ccccc2Nc2ccc(-c3cccc4ccccc34)cc2-c2ccccc2)cc1 ; c1ccc(-c2ccccc2N(c2ccc(-c3ccccc3-n3c4ccccc4c4ccccc43)cc2)c2ccc(-c3cccc4ccccc34)cc2-c2ccccc2)cc1]

Compound subA-11 (10 g, 28.3 mmol), Compound amine29 (13.3 g, 29.7 mmol) and sodium tert-butoxide (3.5 g, 36.7 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 15.3 g of Compound 2-30. (Yield: 71%, MS: [M+H]⁺=765)

### Synthesis Example 2-31

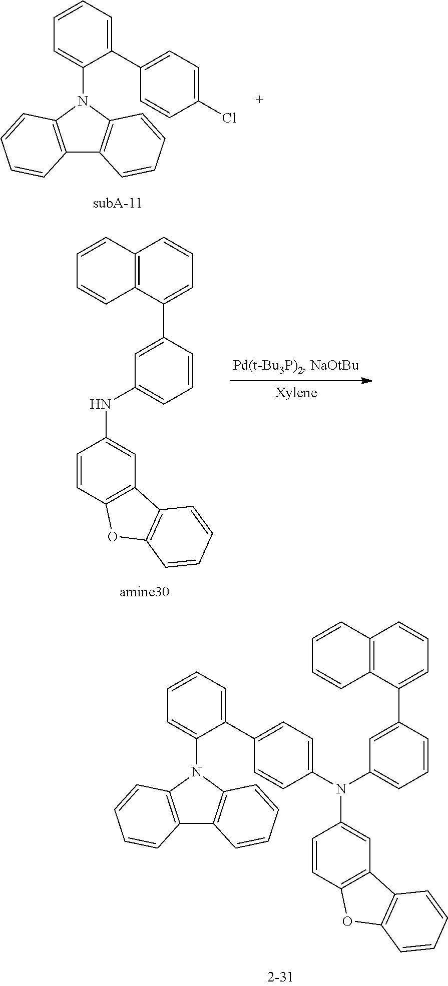 [SMILES: Clc1ccc(-c2ccccc2-n2c3ccccc3c3ccccc32)cc1 ; c1cc(Nc2ccc3oc4ccccc4c3c2)cc(-c2cccc3ccccc23)c1 ; c1cc(-c2cccc3ccccc23)cc(N(c2ccc(-c3ccccc3-n3c4ccccc4c4ccccc43)cc2)c2ccc3oc4ccccc4c3c2)c1]

Compound subA-11 (10 g, 28.3 mmol), Compound amine30 (11.4 g, 29.7 mmol) and sodium tert-butoxide (3.5 g, 36.7 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.1 g of Compound 2-31. (Yield: 66%, MS: [M+H]⁺=703)

### Synthesis Example 2-32

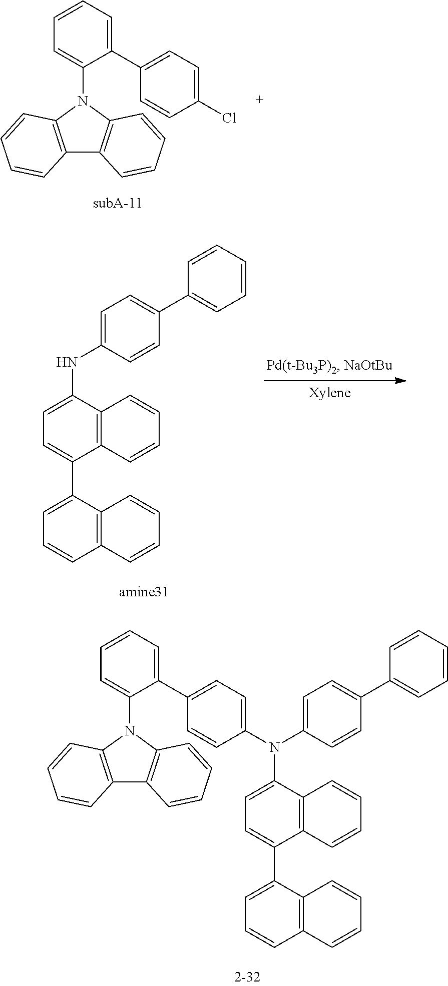 [SMILES: Clc1ccc(-c2ccccc2-n2c3ccccc3c3ccccc32)cc1 ; c1ccc(-c2ccc(Nc3ccc(-c4cccc5ccccc45)c4ccccc34)cc2)cc1 ; c1ccc(-c2ccc(N(c3ccc(-c4ccccc4-n4c5ccccc5c5ccccc54)cc3)c3ccc(-c4cccc5ccccc45)c4ccccc34)cc2)cc1]

Compound subA-11 (10 g, 28.3 mmol), Compound amine31 (12.5 g, 29.7 mmol) and sodium tert-butoxide (3.5 g, 36.7 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 14.2 g of Compound 2-32. (Yield: 68%, MS: [M+H]⁺=739)

### Synthesis Example 2-33

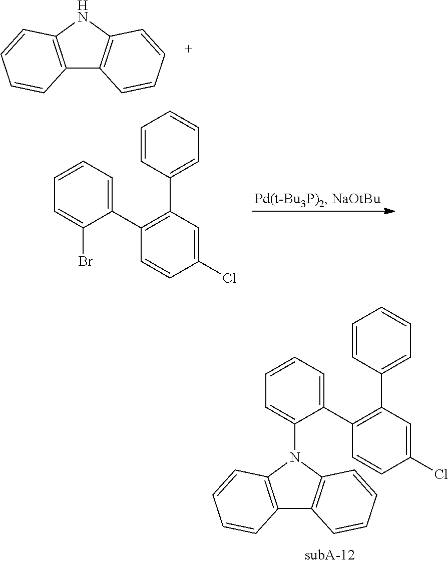

9H-carbazole (10 g, 59.8 mmol), 2-bromo-4′-chloro-1,1′:2′,1″-terphenyl (21.6 g, 62.8 mmol) and sodium tert-butoxide (7.5 g, 77.7 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 16.9 g of Compound subA-12. (Yield: 66%, MS: [M+H]⁺=430)

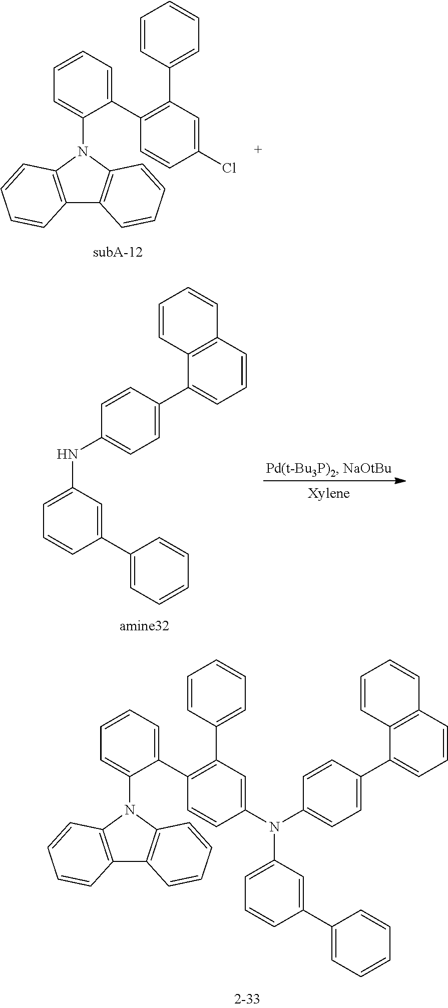 [SMILES: Clc1ccc(-c2ccccc2-n2c3ccccc3c3ccccc32)c(-c2ccccc2)c1 ; c1ccc(-c2cccc(Nc3ccc(-c4cccc5ccccc45)cc3)c2)cc1 ; c1ccc(-c2cccc(N(c3ccc(-c4cccc5ccccc45)cc3)c3ccc(-c4ccccc4-n4c5ccccc5c5ccccc54)c(-c4ccccc4)c3)c2)cc1]

Compound subA-12 (10 g, 23.3 mmol), Compound amine32 (9.1 g, 24.4 mmol) and sodium tert-butoxide (2.9 g, 30.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 12.6 g of Compound 2-33. (Yield: 71%, MS: [M+H]⁺=765)

### Synthesis Example 2-34

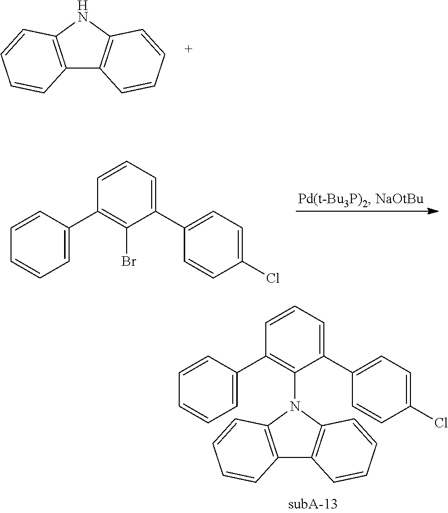

9H-carbazole (10 g, 59.8 mmol), 2′-bromo-4-chloro-1,1′:3′,1″-terphenyl (21.6 g, 62.8 mmol) and sodium tert-butoxide (7.5 g, 77.7 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 16.4 g of Compound subA-13. (Yield: 64%, MS: [M+H]⁺=430)

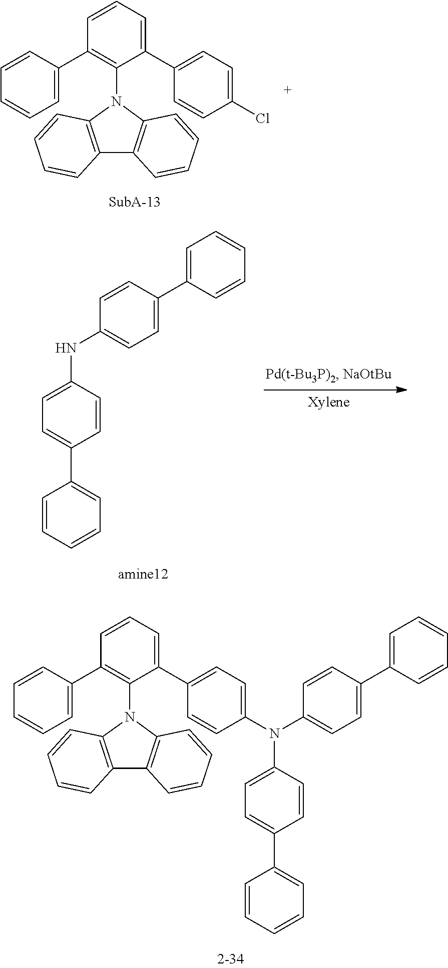 [SMILES: Clc1ccc(-c2cccc(-c3ccccc3)c2-n2c3ccccc3c3ccccc32)cc1 ; c1ccc(-c2ccc(Nc3ccc(-c4ccccc4)cc3)cc2)cc1 ; c1ccc(-c2ccc(N(c3ccc(-c4ccccc4)cc3)c3ccc(-c4cccc(-c5ccccc5)c4-n4c5ccccc5c5ccccc54)cc3)cc2)cc1]

Compound subA-13 (10 g, 23.3 mmol), Compound amine12 (7.8 g, 24.4 mmol) and sodium tert-butoxide (2.9 g, 30.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 11.8 g of Compound 2-34. (Yield: 71%, MS: [M+H]⁺=715)

### Synthesis Example 2-35

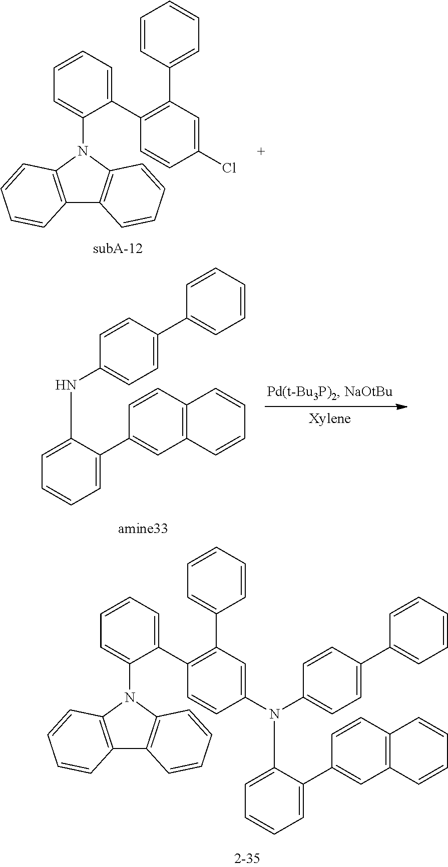 [SMILES: Clc1ccc(-c2ccccc2-n2c3ccccc3c3ccccc32)c(-c2ccccc2)c1 ; c1ccc(-c2ccc(Nc3ccccc3-c3ccc4ccccc4c3)cc2)cc1 ; c1ccc(-c2ccc(N(c3ccc(-c4ccccc4-n4c5ccccc5c5ccccc54)c(-c4ccccc4)c3)c3ccccc3-c3ccc4ccccc4c3)cc2)cc1]

Compound subA-12 (10 g, 23.3 mmol), Compound amine33 (9.1 g, 24.4 mmol) and sodium tert-butoxide (2.9 g, 30.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 12.3 g of Compound 2-35. (Yield: 69%, MS: [M+H]⁺=765)

### Synthesis Example 2-36

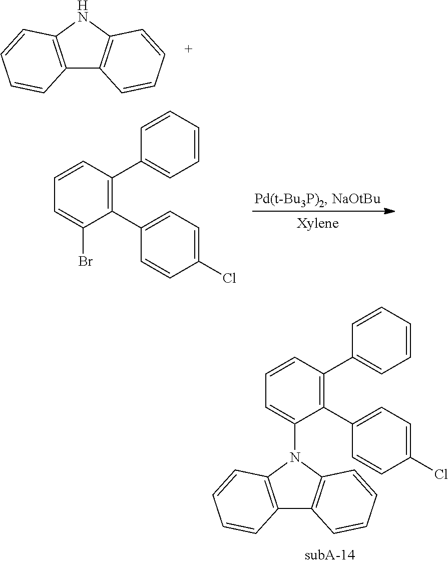

9H-carbazole (10 g, 59.8 mmol), 3′-bromo-4″-chloro-1,1′:2′,1″-terphenyl (21.6 g, 62.8 mmol) and sodium tert-butoxide (7.5 g, 77.7 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 15.7 g of Compound subA-14. (Yield: 61%, MS: [M+H]⁺=430)

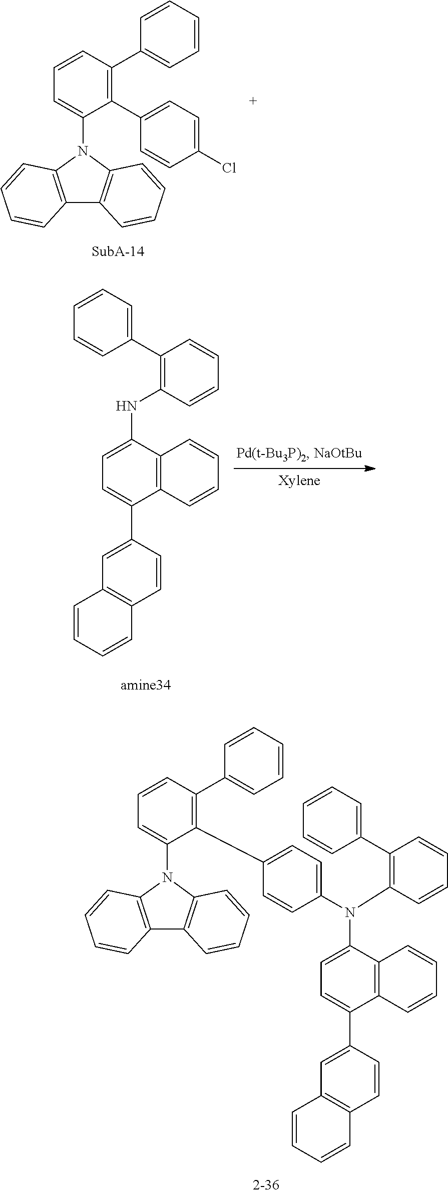 [SMILES: Clc1ccc(-c2c(-c3ccccc3)cccc2-n2c3ccccc3c3ccccc32)cc1 ; c1ccc(-c2ccccc2Nc2ccc(-c3ccc4ccccc4c3)c3ccccc23)cc1 ; c1ccc(-c2ccccc2N(c2ccc(-c3c(-c4ccccc4)cccc3-n3c4ccccc4c4ccccc43)cc2)c2ccc(-c3ccc4ccccc4c3)c3ccccc23)cc1]

Compound subA-14 (10 g, 23.3 mmol), Compound amine34 (10.3 g, 24.4 mmol) and sodium tert-butoxide (2.9 g, 30.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.6 g of Compound 2-36. (Yield: 72%, MS: [M+H]⁺=815)

### Synthesis Example 2-37

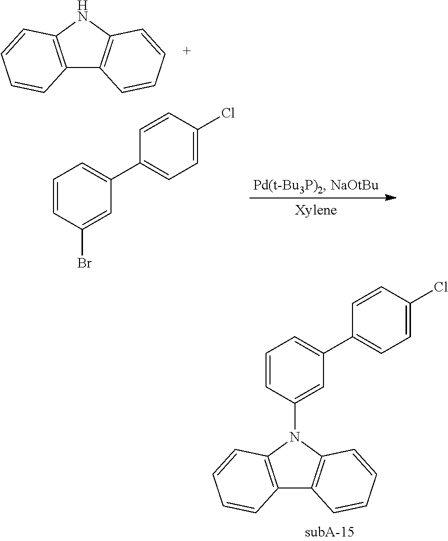

9H-carbazole (10 g, 59.8 mmol), 3-bromo-4′-chloro-1,1′-biphenyl (16.8 g, 62.8 mmol) and sodium tert-butoxide (7.5 g, 77.7 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.3 g of Compound subA-15. (Yield: 63%, MS: [M+H]⁺=354)

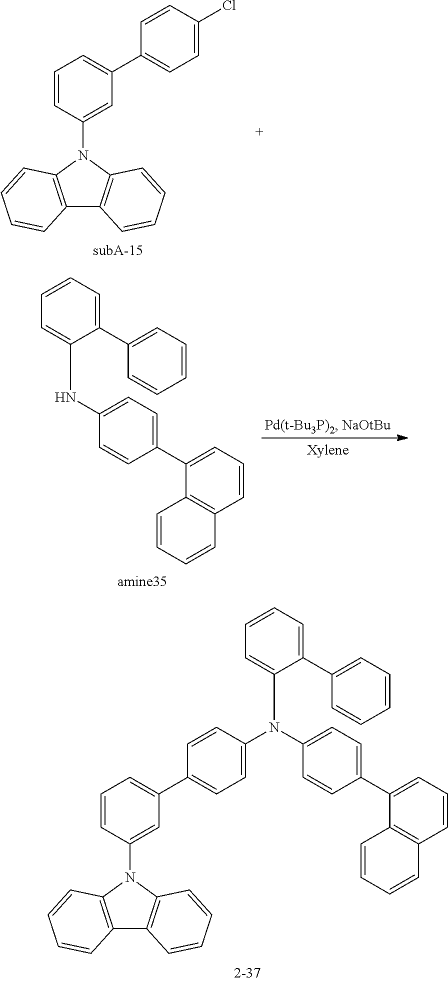 [SMILES: Clc1ccc(-c2cccc(-n3c4ccccc4c4ccccc43)c2)cc1 ; c1ccc(-c2ccccc2Nc2ccc(-c3cccc4ccccc34)cc2)cc1 ; c1ccc(-c2ccccc2N(c2ccc(-c3cccc(-n4c5ccccc5c5ccccc54)c3)cc2)c2ccc(-c3cccc4ccccc34)cc2)cc1]

Compound subA-15 (10 g, 28.3 mmol), Compound amine35 (11.0 g, 29.7 mmol) and sodium tert-butoxide (3.5 g, 36.7 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.2 g of Compound 2-37. (Yield: 68%, MS: [M+H]⁺=689)

### Synthesis Example 2-38

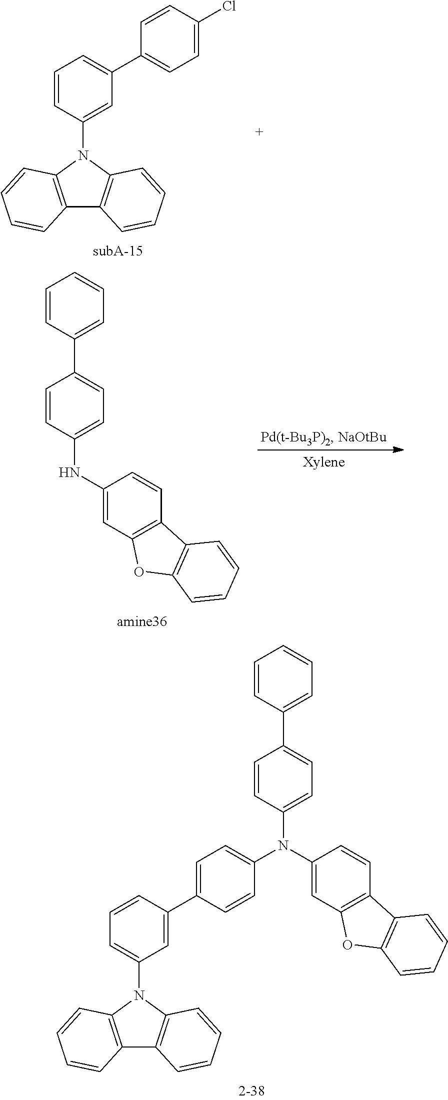 [SMILES: Clc1ccc(-c2cccc(-n3c4ccccc4c4ccccc43)c2)cc1 ; c1ccc(-c2ccc(Nc3ccc4c(c3)oc3ccccc34)cc2)cc1 ; c1ccc(-c2ccc(N(c3ccc(-c4cccc(-n5c6ccccc6c6ccccc65)c4)cc3)c3ccc4c(c3)oc3ccccc34)cc2)cc1]

Compound subA-15 (10 g, 28.3 mmol), Compound amine36 (10.0 g, 29.7 mmol) and sodium tert-butoxide (3.5 g, 36.7 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 12.7 g of Compound 2-38. (Yield: 69%, MS: [M+H]⁺=653)

### Synthesis Example 2-39

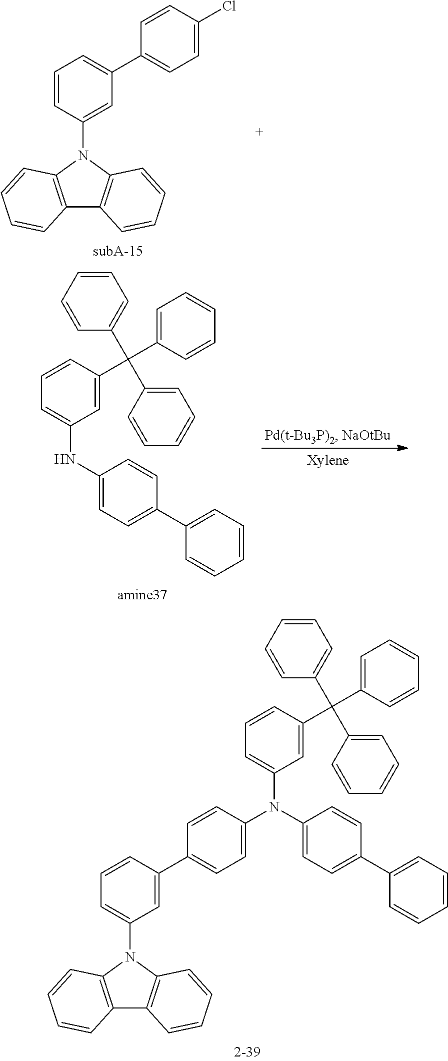

Compound subA-15 (10 g, 28.3 mmol), Compound amine37 (14.5 g, 29.7 mmol) and sodium tert-butoxide (3.5 g, 36.7 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.7 g of Compound 2-39. (Yield: 60%, MS: [M+H]⁺=805)

### Synthesis Example 2-40

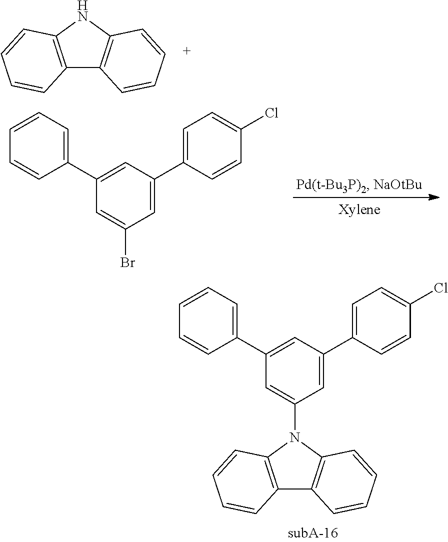

9H-carbazole (10 g, 59.8 mmol), 5′-bromo-4-chloro-1,1′:3′,1″-terphenyl (21.6 g, 62.8 mmol) and sodium tert-butoxide (7.5 g, 77.7 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 18 g of Compound subA-16. (Yield: 70%, MS: [M+H]⁺=430)

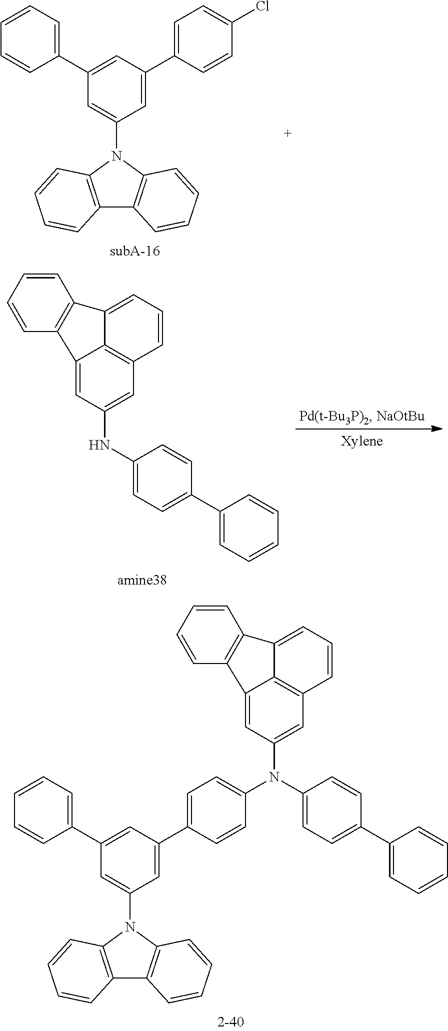

Compound subA-16 (10 g, 23.3 mmol), Compound amine38 (9 g, 24.4 mmol) and sodium tert-butoxide (2.9 g, 30.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 12.6 g of Compound 2-40. (Yield: 71%, MS: [M+H]⁺=763)

### Synthesis Example 2-41

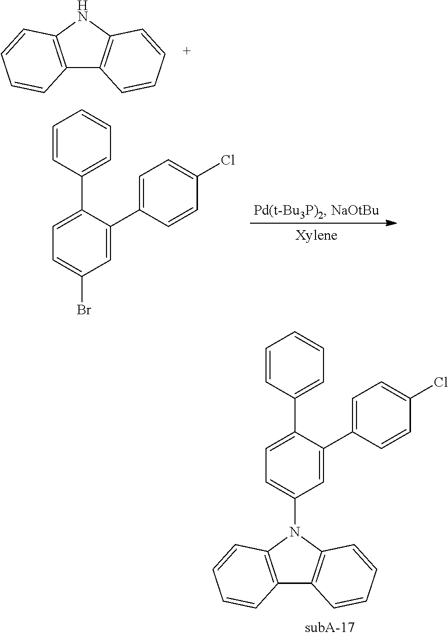

9H-carbazole (10 g, 59.8 mmol), 5′-bromo-4-chloro-1,1′:2′,1″-terphenyl (21.6 g, 62.8 mmol) and sodium tert-butoxide (7.5 g, 77.7 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 15.4 g of Compound subA-17. (Yield: 60%, MS: [M+H]⁺=430)

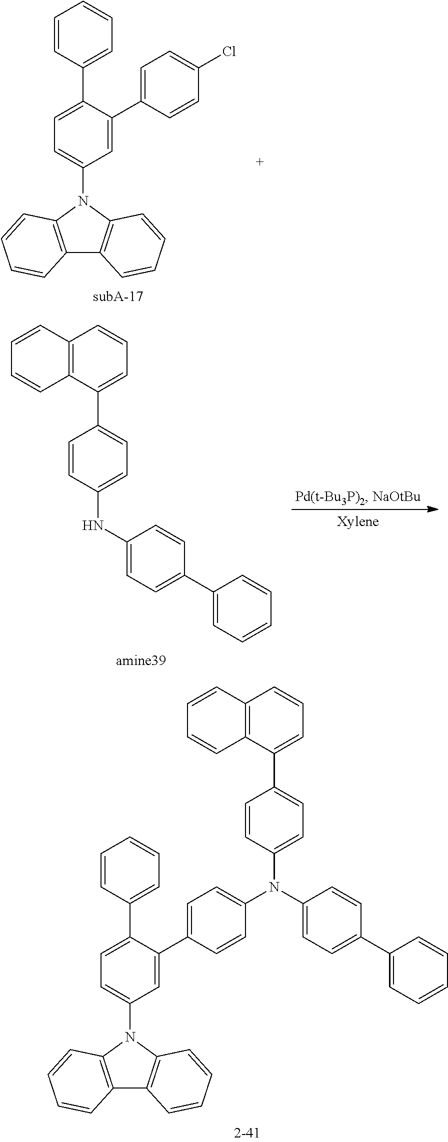 [SMILES: Clc1ccc(-c2cc(-n3c4ccccc4c4ccccc43)ccc2-c2ccccc2)cc1 ; c1ccc(-c2ccc(Nc3ccc(-c4cccc5ccccc45)cc3)cc2)cc1 ; c1ccc(-c2ccc(N(c3ccc(-c4cc(-n5c6ccccc6c6ccccc65)ccc4-c4ccccc4)cc3)c3ccc(-c4cccc5ccccc45)cc3)cc2)cc1]

Compound subA-17 (10 g, 23.3 mmol), Compound amine39 (9.1 g, 24.4 mmol) and sodium tert-butoxide (2.9 g, 30.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 11.7 g of Compound 2-41. (Yield: 66%, MS: [M+H]⁺=766)

### Synthesis Example 2-42

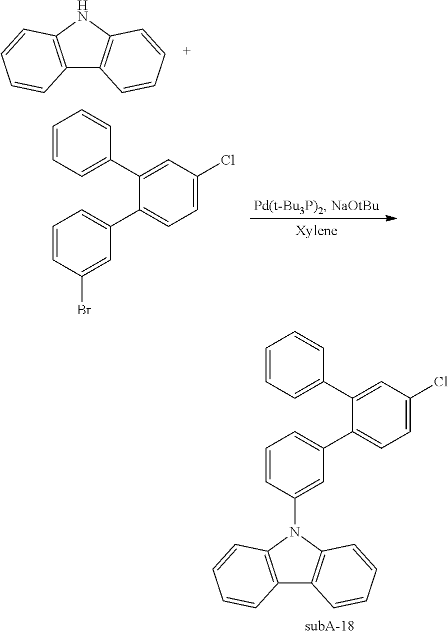

9H-carbazole (10 g, 59.8 mmol), 3-bromo-4′-chloro-1,1′:2′,1″-terphenyl (21.6 g, 62.8 mmol) and sodium tert-butoxide (7.5 g, 77.7 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 19 g of Compound subA-18. (Yield: 74%, MS: [M+H]⁺=430)

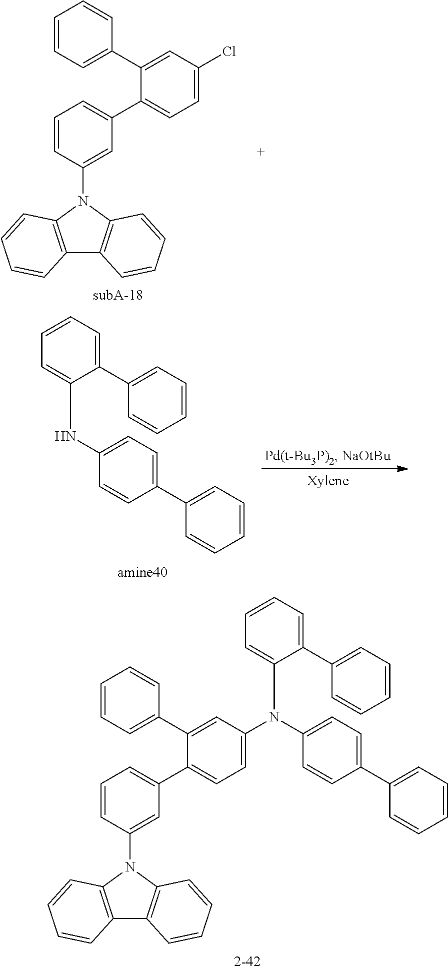 [SMILES: Clc1ccc(-c2cccc(-n3c4ccccc4c4ccccc43)c2)c(-c2ccccc2)c1 ; c1ccc(-c2ccc(Nc3ccccc3-c3ccccc3)cc2)cc1 ; c1ccc(-c2ccc(N(c3ccc(-c4cccc(-n5c6ccccc6c6ccccc65)c4)c(-c4ccccc4)c3)c3ccccc3-c3ccccc3)cc2)cc1]

Compound subA-18 (10 g, 23.3 mmol), Compound amine40 (7.8 g, 24.4 mmol) and sodium tert-butoxide (2.9 g, 30.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 12.1 g of Compound 2-42. (Yield: 73%, MS: [M+H]⁺=715)

### Synthesis Example 2-43

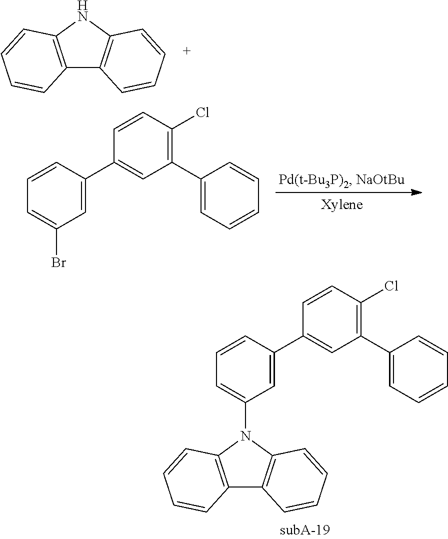

9H-carbazole (10 g, 59.8 mmol), 3-bromo-4′-chloro-1,1′:3′,1″-terphenyl (21.6 g, 62.8 mmol) and sodium tert-butoxide (7.5 g, 77.7 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 16.7 g of Compound subA-19. (Yield: 65%, MS: [M+H]⁺=430).

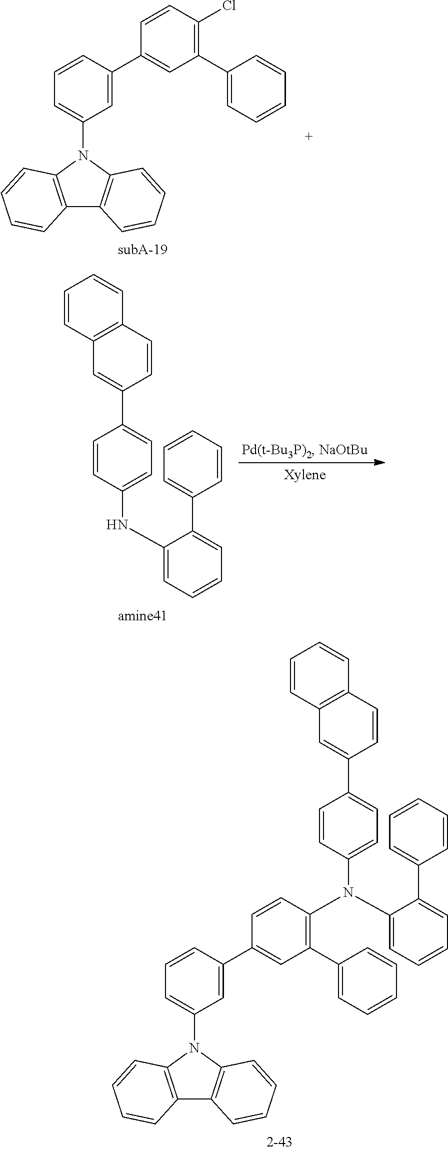 [SMILES: Clc1ccc(-c2cccc(-n3c4ccccc4c4ccccc43)c2)cc1-c1ccccc1 ; c1ccc(-c2ccccc2Nc2ccc(-c3ccc4ccccc4c3)cc2)cc1 ; c1ccc(-c2ccccc2N(c2ccc(-c3ccc4ccccc4c3)cc2)c2ccc(-c3cccc(-n4c5ccccc5c5ccccc54)c3)cc2-c2ccccc2)cc1]

Compound subA-19 (10 g, 23.3 mmol), Compound amine41 (9.1 g, 24.4 mmol) and sodium tert-butoxide (2.9 g, 30.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 12.6 g of Compound 2-43. (Yield: 71%, MS: [M+H]⁺=765)

### Synthesis Example 2-44

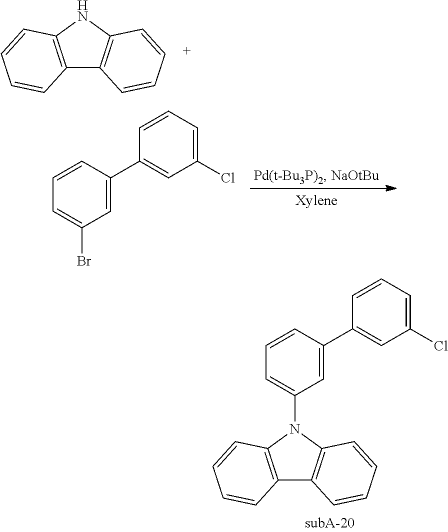

9H-carbazole (10 g, 59.8 mmol), 3-bromo-3′-chloro-1,1′-biphenyl (16.8 g, 62.8 mmol) and sodium tert-butoxide (7.5 g, 77.7 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 14.1 g of Compound subA-20. (Yield: 67%, MS: [M+H]⁺=354)

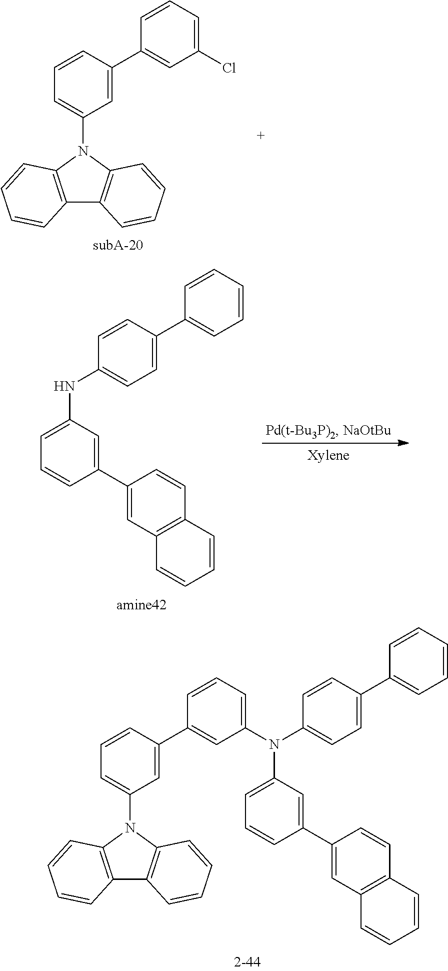 [SMILES: Clc1cccc(-c2cccc(-n3c4ccccc4c4ccccc43)c2)c1 ; c1ccc(-c2ccc(Nc3cccc(-c4ccc5ccccc5c4)c3)cc2)cc1 ; c1ccc(-c2ccc(N(c3cccc(-c4cccc(-n5c6ccccc6c6ccccc65)c4)c3)c3cccc(-c4ccc5ccccc5c4)c3)cc2)cc1]

Compound subA-20 (10 g, 28.3 mmol), Compound amine42 (11.1 g, 29.7 mmol) and sodium tert-butoxide (3.5 g, 36.7 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 12.1 g of Compound 2-44. (Yield: 62%, MS: [M+H]⁺=689)

### Synthesis Example 2-45

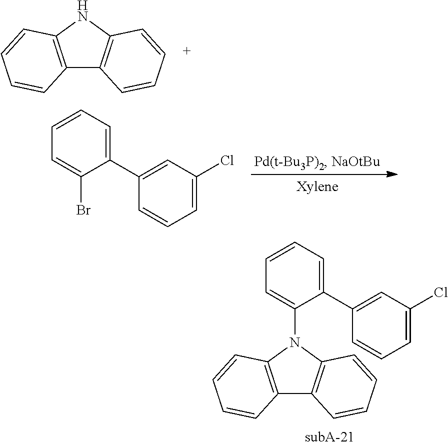

9H-carbazole (10 g, 59.8 mmol), 2-bromo-3′-chloro-1,1′-biphenyl (16.8 g, 62.8 mmol) and sodium tert-butoxide (7.5 g, 77.7 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.3 g of Compound subA-21. (Yield: 63%, MS: [M+H]⁺=354)

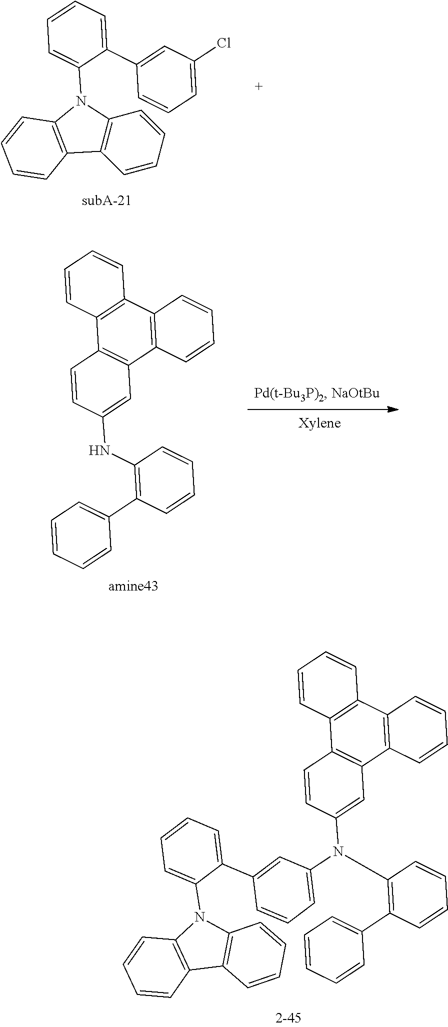 [SMILES: Clc1cccc(-c2ccccc2-n2c3ccccc3c3ccccc32)c1 ; c1ccc(-c2ccccc2Nc2ccc3c4ccccc4c4ccccc4c3c2)cc1 ; c1ccc(-c2ccccc2N(c2cccc(-c3ccccc3-n3c4ccccc4c4ccccc43)c2)c2ccc3c4ccccc4c4ccccc4c3c2)cc1]

Compound subA-21 (10 g, 28.3 mmol), Compound amine43 (11.7 g, 29.7 mmol) and sodium tert-butoxide (3.5 g, 36.7 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 14.7 g of Compound 2-45. (Yield: 73%, MS: [M+H]⁺=713)

### Synthesis Example 2-46

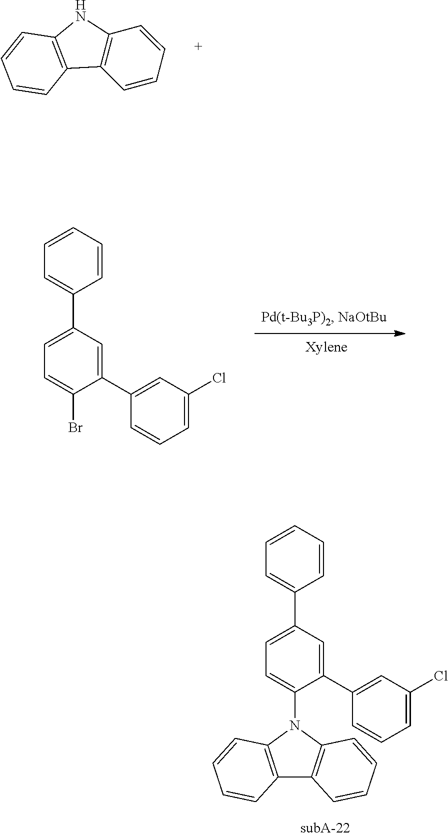 [SMILES: c1ccc2c(c1)[nH]c1ccccc12 ; Clc1cccc(-c2cc(-c3ccccc3)ccc2Br)c1 ; Clc1cccc(-c2cc(-c3ccccc3)ccc2-n2c3ccccc3c3ccccc32)c1]

9H-carbazole (10 g, 59.8 mmol), 6′-bromo-3-chloro-1,1′:3′,1″-terphenyl (21.6 g, 62.8 mmol) and sodium tert-butoxide (7.5 g, 77.7 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 16.4 g of Compound subA-22. (Yield: 64%, MS: [M+H]⁺=430)

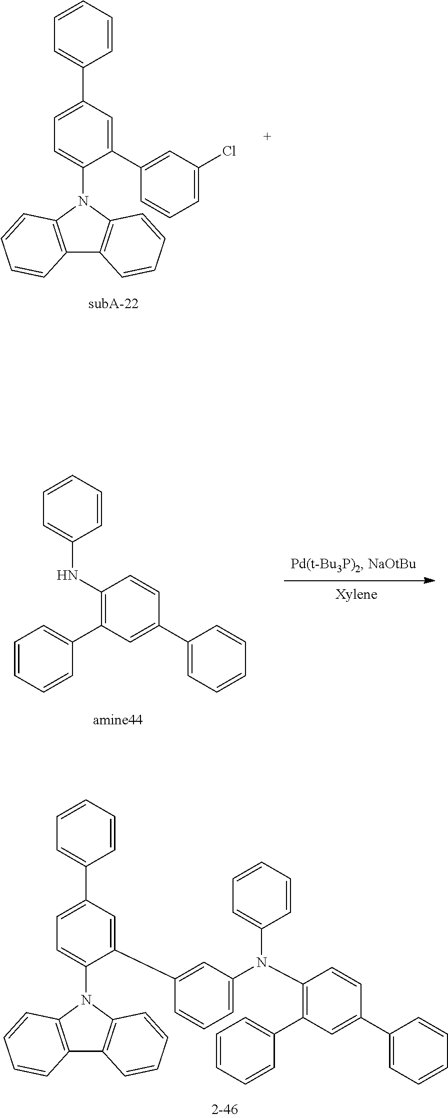 [SMILES: Clc1cccc(-c2cc(-c3ccccc3)ccc2-n2c3ccccc3c3ccccc32)c1 ; c1ccc(Nc2ccc(-c3ccccc3)cc2-c2ccccc2)cc1 ; c1ccc(-c2ccc(N(c3ccccc3)c3cccc(-c4cc(-c5ccccc5)ccc4-n4c5ccccc5c5ccccc54)c3)c(-c3ccccc3)c2)cc1]

Compound subA-22 (10 g, 23.3 mmol), Compound amine44 (7.8 g, 24.4 mmol) and sodium tert-butoxide (2.9 g, 30.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 10.1 g of Compound 2-46. (Yield: 61%, MS: [M+H]⁺=715)

### Synthesis Example 2-47

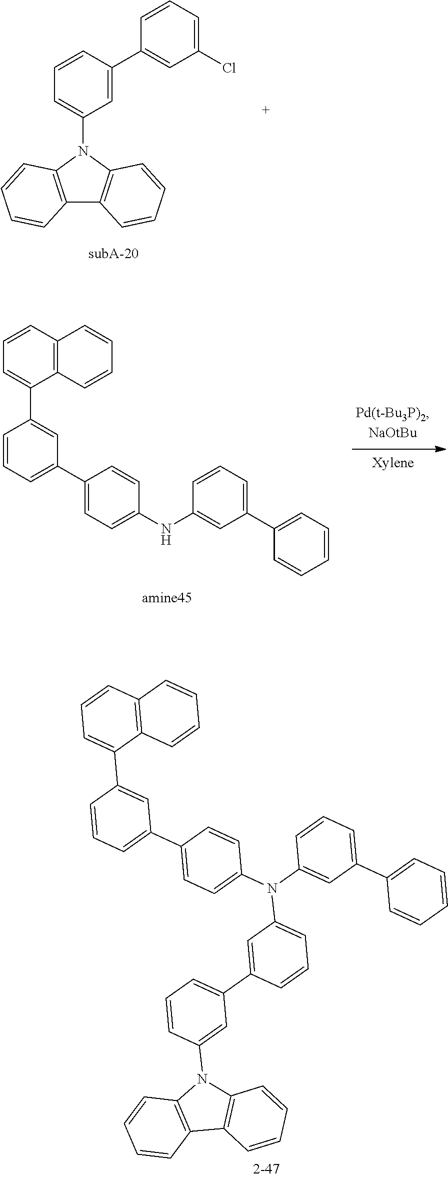 [SMILES: Clc1cccc(-c2cccc(-n3c4ccccc4c4ccccc43)c2)c1 ; c1ccc(-c2cccc(Nc3ccc(-c4cccc(-c5cccc6ccccc56)c4)cc3)c2)cc1 ; c1ccc(-c2cccc(N(c3ccc(-c4cccc(-c5cccc6ccccc56)c4)cc3)c3cccc(-c4cccc(-n5c6ccccc6c6ccccc65)c4)c3)c2)cc1]

Compound subA-20 (10 g, 28.3 mmol), Compound amine45 (13.3 g, 29.7 mmol) and sodium tert-butoxide (3.5 g, 36.7 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.8 g of Compound 2-47. (Yield: 64%, MS: [M+H]⁺=765)

### Synthesis Example 2-48

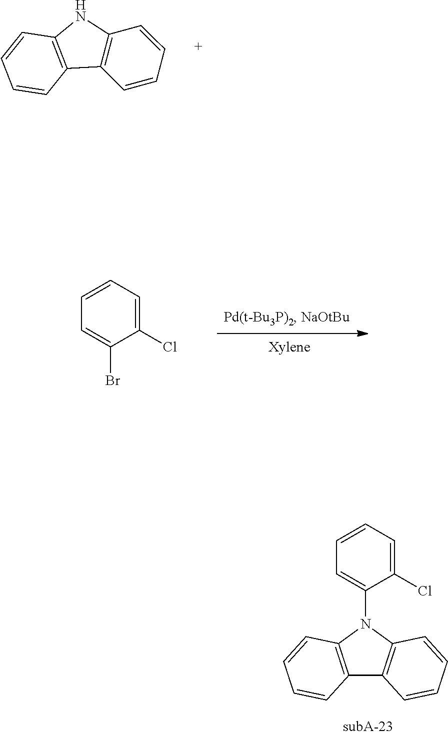 [SMILES: c1ccc2c(c1)[nH]c1ccccc12 ; Clc1ccccc1Br ; Clc1ccccc1-n1c2ccccc2c2ccccc21]

9H-carbazole (10 g, 59.8 mmol), 1-bromo-2-chlorobenzene (12 g, 62.8 mmol) and sodium tert-butoxide (7.5 g, 77.7 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 11.9 g of Compound subA-23. (Yield: 72%, MS: [M+H]⁺=278)

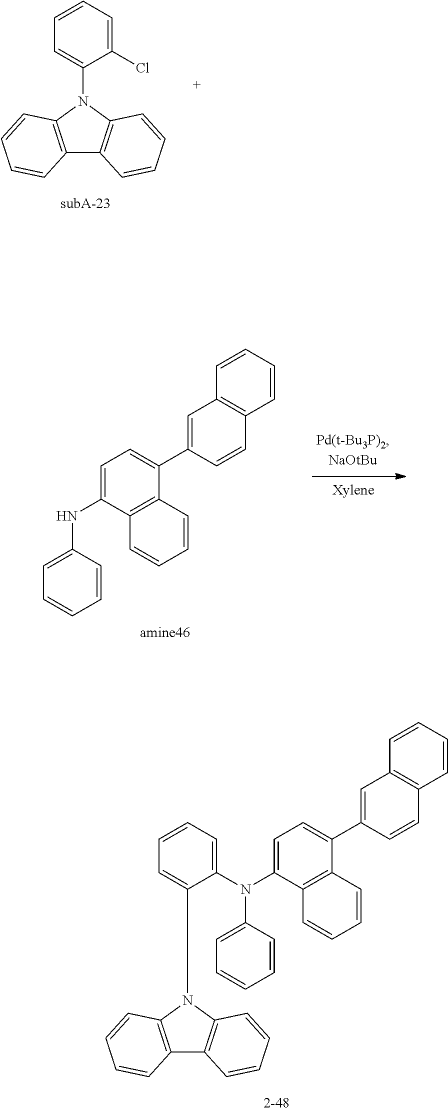 [SMILES: Clc1ccccc1-n1c2ccccc2c2ccccc21 ; c1ccc(Nc2ccc(-c3ccc4ccccc4c3)c3ccccc23)cc1 ; c1ccc(N(c2ccccc2-n2c3ccccc3c3ccccc32)c2ccc(-c3ccc4ccccc4c3)c3ccccc23)cc1]

Compound subA-23 (10 g, 36 mmol), Compound amine46 (13.1 g, 37.8 mmol) and sodium tert-butoxide (4.5 g, 46.8 mmol) were added to 200 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.9 g of Compound 2-48. (Yield: 66%, MS: [M+H]⁺=587)

### Synthesis Example 2-49

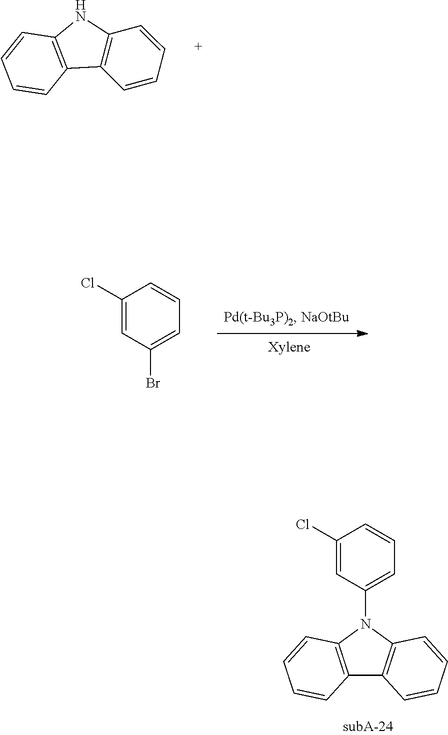 [SMILES: c1ccc2c(c1)[nH]c1ccccc12 ; Clc1cccc(Br)c1 ; Clc1cccc(-n2c3ccccc3c3ccccc32)c1]

9H-carbazole (10 g, 59.8 mmol), 1-bromo-3-chlorobenzene (12 g, 62.8 mmol) and sodium tert-butoxide (7.5 g, 77.7 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 9.9 g of Compound subA-24. (Yield: 60%, MS: [M+H]⁺=278)

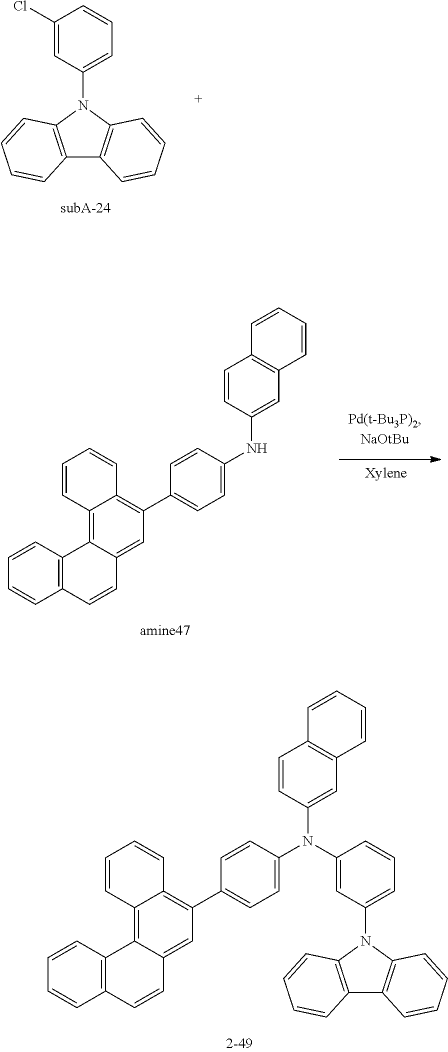 [SMILES: Clc1cccc(-n2c3ccccc3c3ccccc32)c1 ; c1ccc2cc(Nc3ccc(-c4cc5ccc6ccccc6c5c5ccccc45)cc3)ccc2c1 ; c1cc(N(c2ccc(-c3cc4ccc5ccccc5c4c4ccccc34)cc2)c2ccc3ccccc3c2)cc(-n2c3ccccc3c3ccccc32)c1]

Compound subA-24 (10 g, 36 mmol), Compound amine47 (16.8 g, 37.8 mmol) and sodium tert-butoxide (4.5 g, 46.8 mmol) were added to 200 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 18 g of Compound 2-49. (Yield: 73%, MS: [M+H]⁺=687)

### Synthesis Example 2-50

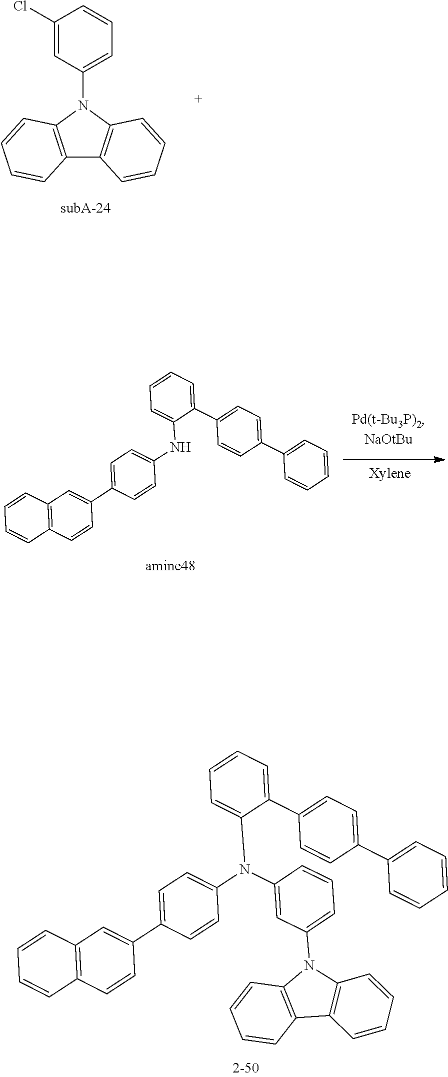 [SMILES: Clc1cccc(-n2c3ccccc3c3ccccc32)c1 ; c1ccc(-c2ccc(-c3ccccc3Nc3ccc(-c4ccc5ccccc5c4)cc3)cc2)cc1 ; c1ccc(-c2ccc(-c3ccccc3N(c3ccc(-c4ccc5ccccc5c4)cc3)c3cccc(-n4c5ccccc5c5ccccc54)c3)cc2)cc1]

Compound subA-24 (10 g, 36 mmol), Compound amine48 (16.9 g, 37.8 mmol) and sodium tert-butoxide (4.5 g, 46.8 mmol) were added to 200 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 17 g of Compound 2-50. (Yield: 69%, MS: [M+H]⁺=687)

### Synthesis Example 2-51

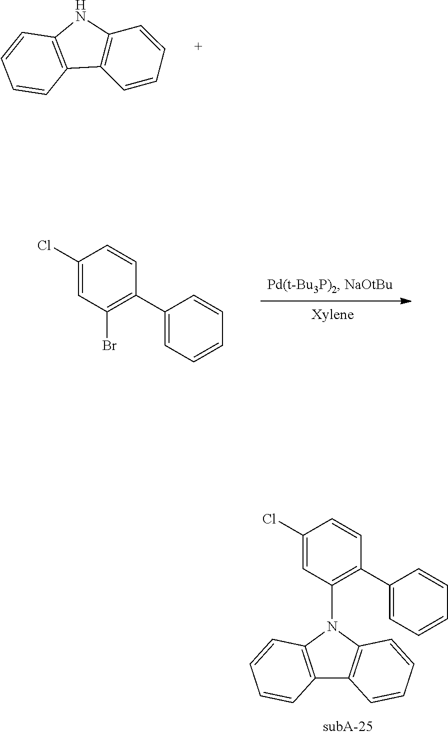 [SMILES: c1ccc2c(c1)[nH]c1ccccc12 ; Clc1ccc(-c2ccccc2)c(Br)c1 ; Clc1ccc(-c2ccccc2)c(-n2c3ccccc3c3ccccc32)c1]

9H-carbazole (10 g, 59.8 mmol), 2-bromo-4-chloro-1,1′-biphenyl (16.8 g, 62.8 mmol) and sodium tert-butoxide (7.5 g, 77.7 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 15.8 g of Compound subA-25. (Yield: 75%, MS: [M+H]⁺=354)

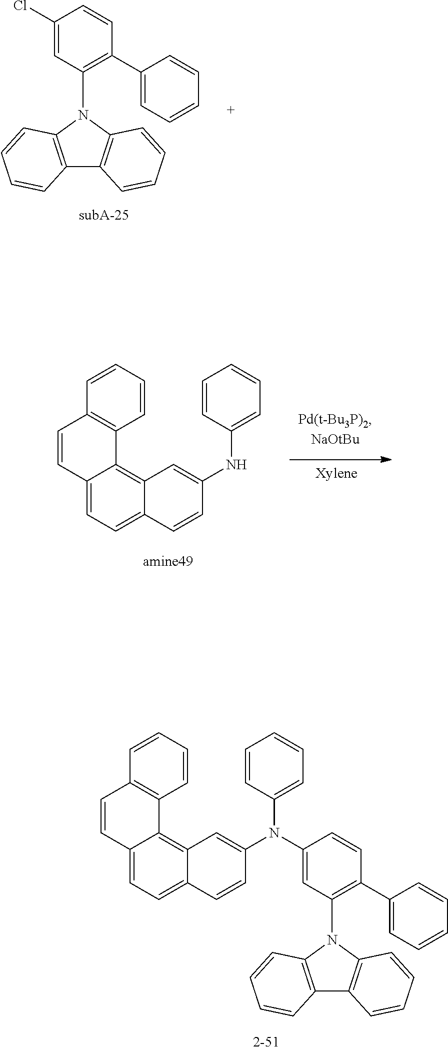 [SMILES: Clc1ccc(-c2ccccc2)c(-n2c3ccccc3c3ccccc32)c1 ; c1ccc(Nc2ccc3ccc4ccc5ccccc5c4c3c2)cc1 ; c1ccc(-c2ccc(N(c3ccccc3)c3ccc4ccc5ccc6ccccc6c5c4c3)cc2-n2c3ccccc3c3ccccc32)cc1]

Compound subA-25 (10 g, 28.3 mmol), Compound amine49 (9.5 g, 29.7 mmol) and sodium tert-butoxide (3.5 g, 36.7 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.5 g of Compound 2-51. (Yield: 75%, MS: [M+H]⁺=637)

### Synthesis Example 2-52

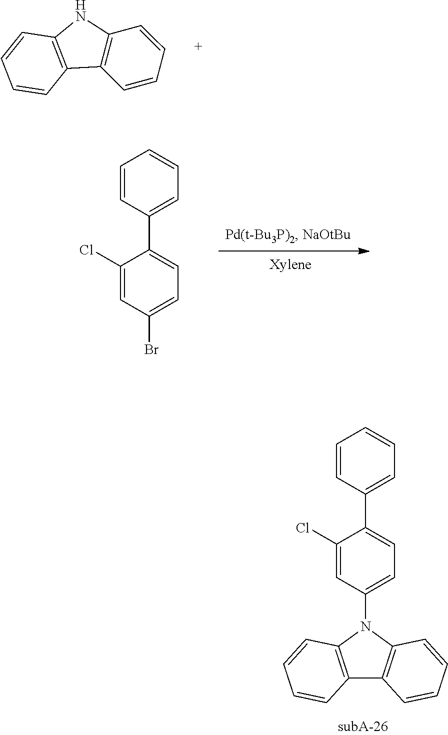 [SMILES: c1ccc2c(c1)[nH]c1ccccc12 ; Clc1cc(Br)ccc1-c1ccccc1 ; Clc1cc(-n2c3ccccc3c3ccccc32)ccc1-c1ccccc1]

9H-carbazole (10 g, 59.8 mmol), 4-bromo-2-chloro-1,1′-biphenyl (16.8 g, 62.8 mmol) and sodium tert-butoxide (7.5 g, 77.7 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 15 g of Compound subA-26. (Yield: 71%, MS: [M+H]⁺=354)

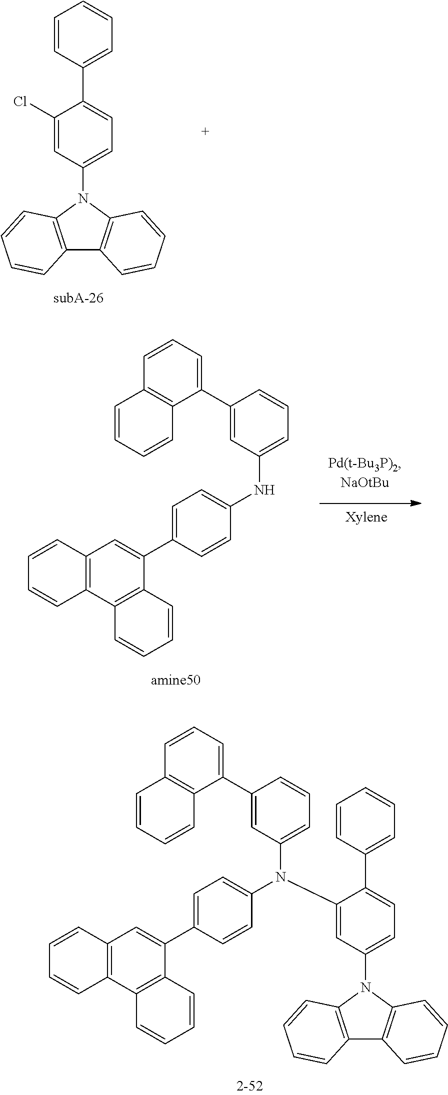 [SMILES: Clc1cc(-n2c3ccccc3c3ccccc32)ccc1-c1ccccc1 ; c1cc(Nc2ccc(-c3cc4ccccc4c4ccccc34)cc2)cc(-c2cccc3ccccc23)c1 ; c1ccc(-c2ccc(-n3c4ccccc4c4ccccc43)cc2N(c2ccc(-c3cc4ccccc4c4ccccc34)cc2)c2cccc(-c3cccc4ccccc34)c2)cc1]

Compound subA-26 (10 g, 28.3 mmol), Compound amine50 (14 g, 29.7 mmol) and sodium tert-butoxide (3.5 g, 36.7 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 14.9 g of Compound 2-52. (Yield: 67%, MS: [M+H]⁺=789)

### Synthesis Example 2-53

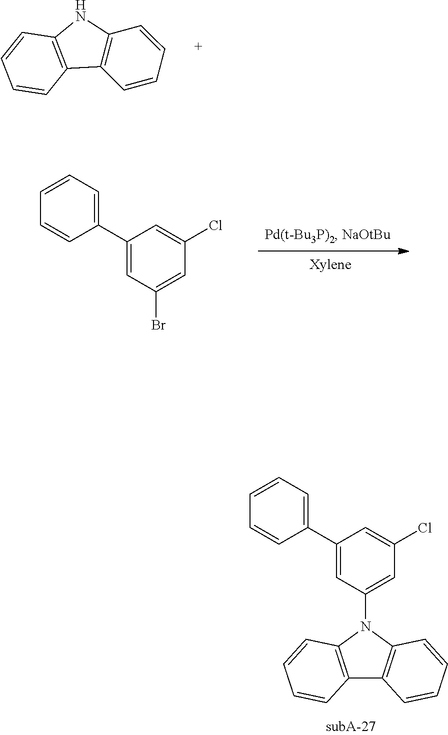 [SMILES: c1ccc2c(c1)[nH]c1ccccc12 ; Clc1cc(Br)cc(-c2ccccc2)c1 ; Clc1cc(-c2ccccc2)cc(-n2c3ccccc3c3ccccc32)c1]

9H-carbazole (10 g, 59.8 mmol), 3-bromo-5-chloro-1,1′-biphenyl (16.8 g, 62.8 mmol) and sodium tert-butoxide (7.5 g, 77.7 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 15.6 g of Compound subA-27. (Yield: 74%, MS: [M+H]⁺=354)

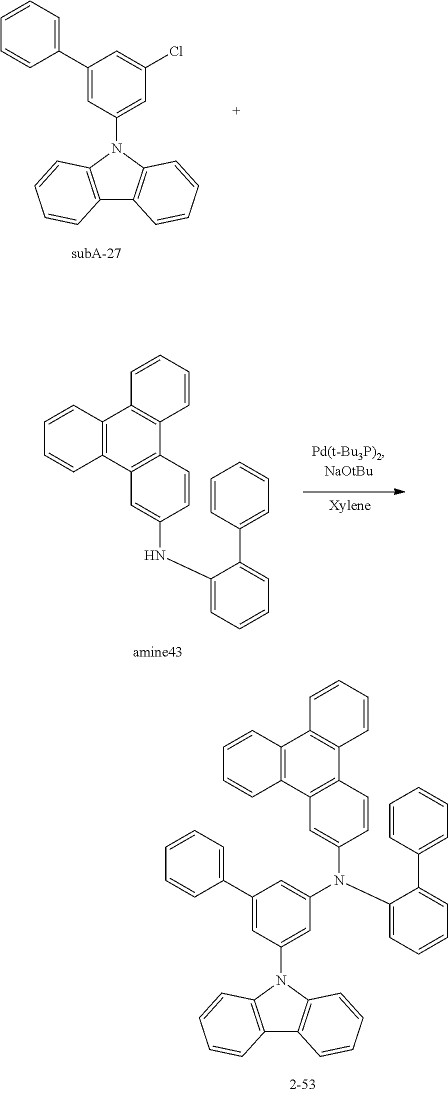 [SMILES: Clc1cc(-c2ccccc2)cc(-n2c3ccccc3c3ccccc32)c1 ; c1ccc(-c2ccccc2Nc2ccc3c4ccccc4c4ccccc4c3c2)cc1 ; c1ccc(-c2cc(N(c3ccc4c5ccccc5c5ccccc5c4c3)c3ccccc3-c3ccccc3)cc(-n3c4ccccc4c4ccccc43)c2)cc1]

Compound subA-27 (10 g, 2.8 mmol), Compound amine43 (1.2 g, 3 mmol) and sodium tert-butoxide (0.4 g, 3.7 mmol) were added to 200 ml of xylene under a nitrogen atmosphere, and the mixture was stirred and refluxed. Then, bis(tri-tert-butylphosphine)palladium(0) (0 g, 0 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 1.2 g of Compound 2-53. (Yield: 60%, MS: [M+H]⁺=713)

### Synthesis Example 2-54

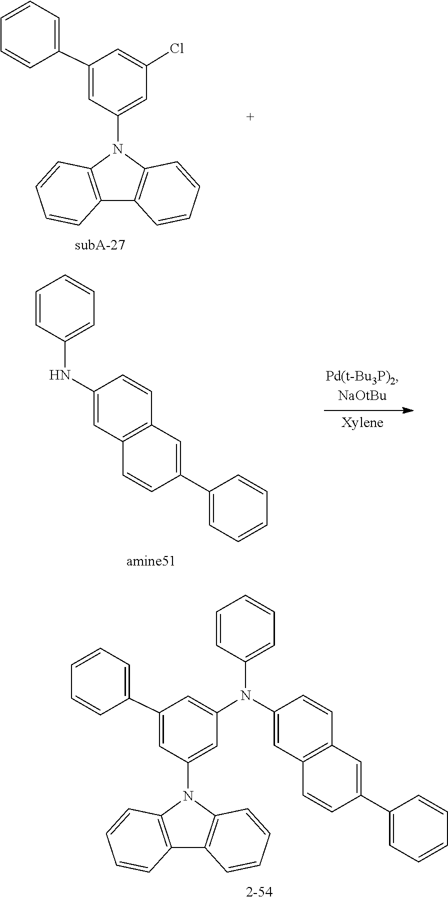 [SMILES: Clc1cc(-c2ccccc2)cc(-n2c3ccccc3c3ccccc32)c1 ; c1ccc(Nc2ccc3cc(-c4ccccc4)ccc3c2)cc1 ; c1ccc(-c2cc(N(c3ccccc3)c3ccc4cc(-c5ccccc5)ccc4c3)cc(-n3c4ccccc4c4ccccc43)c2)cc1]

Compound subA-27 (10 g, 2.8 mmol), Compound amine51 (0.9 g, 3 mmol) and sodium tert-butoxide (0.4 g, 3.7 mmol) were added to 200 ml of xylene under a nitrogen atmosphere, and the mixture was stirred and refluxed. Then, bis(tri-tert-butylphosphine)palladium(0) (0 g, 0 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 1 g of Compound 2-54. (Yield: 60%, MS: [M+H]⁺=613)

### Synthesis Example 2-55

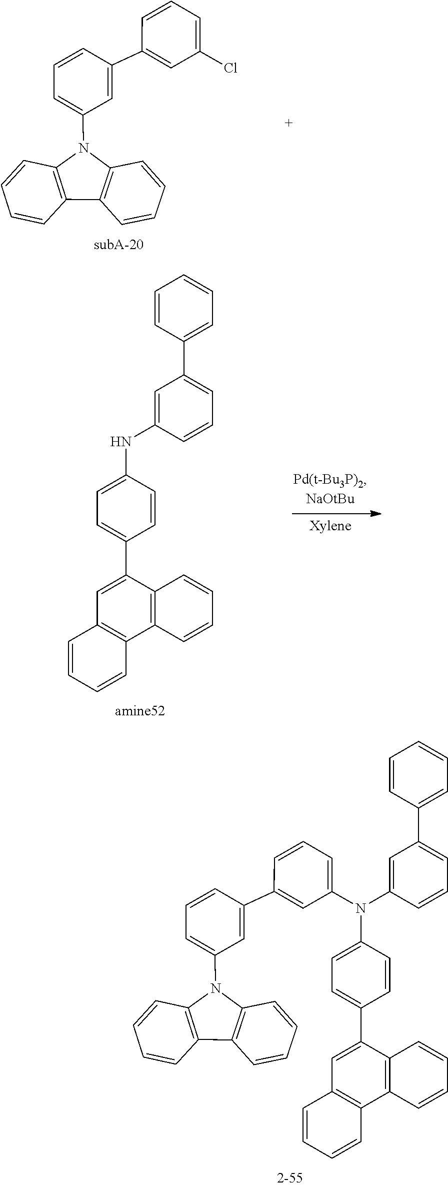 [SMILES: Clc1cccc(-c2cccc(-n3c4ccccc4c4ccccc43)c2)c1 ; c1ccc(-c2cccc(Nc3ccc(-c4cc5ccccc5c5ccccc45)cc3)c2)cc1 ; c1ccc(-c2cccc(N(c3ccc(-c4cc5ccccc5c5ccccc45)cc3)c3cccc(-c4cccc(-n5c6ccccc6c6ccccc65)c4)c3)c2)cc1]

Compound subA-20 (10 g, 28.3 mmol), Compound amine52 (12.5 g, 29.7 mmol) and sodium tert-butoxide (3.5 g, 36.7 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 14.8 g of Compound 2-55. (Yield: 71%, MS: [M+H]⁺=739)

### Synthesis Example 2-56

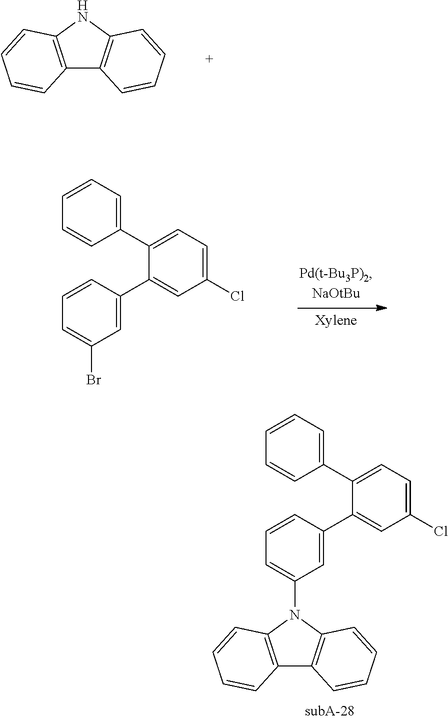 [SMILES: c1ccc2c(c1)[nH]c1ccccc12 ; Clc1ccc(-c2ccccc2)c(-c2cccc(Br)c2)c1 ; Clc1ccc(-c2ccccc2)c(-c2cccc(-n3c4ccccc4c4ccccc43)c2)c1]

9H-carbazole (10 g, 59.8 mmol), 3-bromo-5′-chloro-1,1′:2′,1″-terphenyl (21.6 g, 62.8 mmol) and sodium tert-butoxide (7.5 g, 77.7 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 15.4 g of Compound subA-28. (Yield: 60%, MS: [M+H]⁺=430)

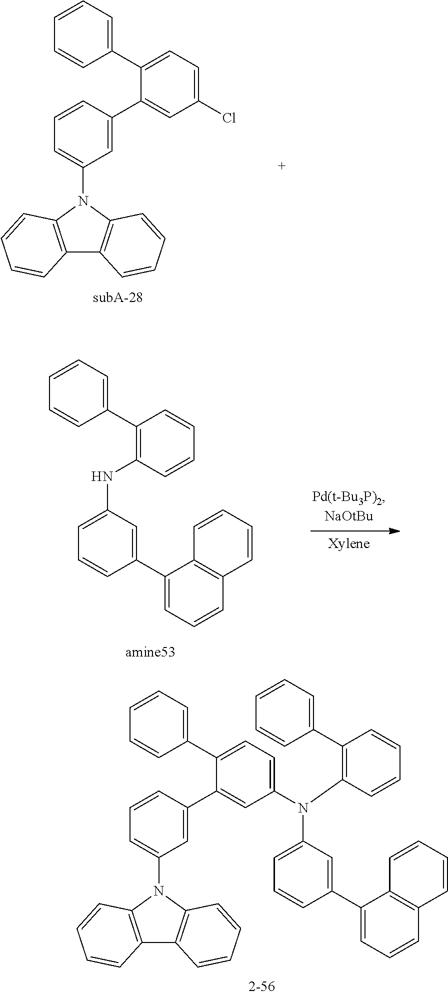 [SMILES: Clc1ccc(-c2ccccc2)c(-c2cccc(-n3c4ccccc4c4ccccc43)c2)c1 ; c1ccc(-c2ccccc2Nc2cccc(-c3cccc4ccccc34)c2)cc1 ; c1ccc(-c2ccc(N(c3cccc(-c4cccc5ccccc45)c3)c3ccccc3-c3ccccc3)cc2-c2cccc(-n3c4ccccc4c4ccccc43)c2)cc1]

Compound subA-28 (10 g, 23.3 mmol), Compound amine53 (9.1 g, 24.4 mmol) and sodium tert-butoxide (2.9 g, 30.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 12.6 g of Compound 2-56. (Yield: 71%, MS: [M+H]⁺=765)

### Synthesis Example 2-57

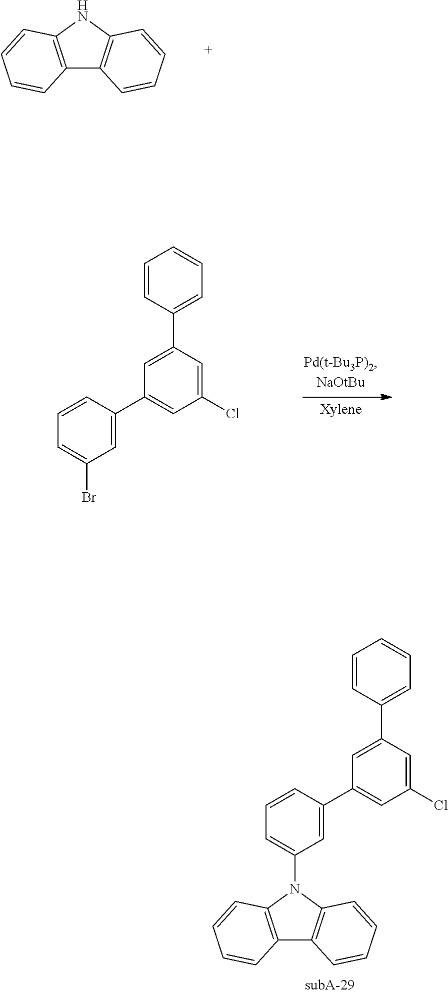 [SMILES: c1ccc2c(c1)[nH]c1ccccc12 ; Clc1cc(-c2ccccc2)cc(-c2cccc(Br)c2)c1 ; Clc1cc(-c2ccccc2)cc(-c2cccc(-n3c4ccccc4c4ccccc43)c2)c1]

9H-carbazole (10 g, 59.8 mmol), 3-bromo-5′-chloro-1,1′:3′,1″-terphenyl (21.6 g, 62.8 mmol) and sodium tert-butoxide (7.5 g, 77.7 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 15.7 g of Compound subA-29. (Yield: 61%, MS: [M+H]⁺=430)

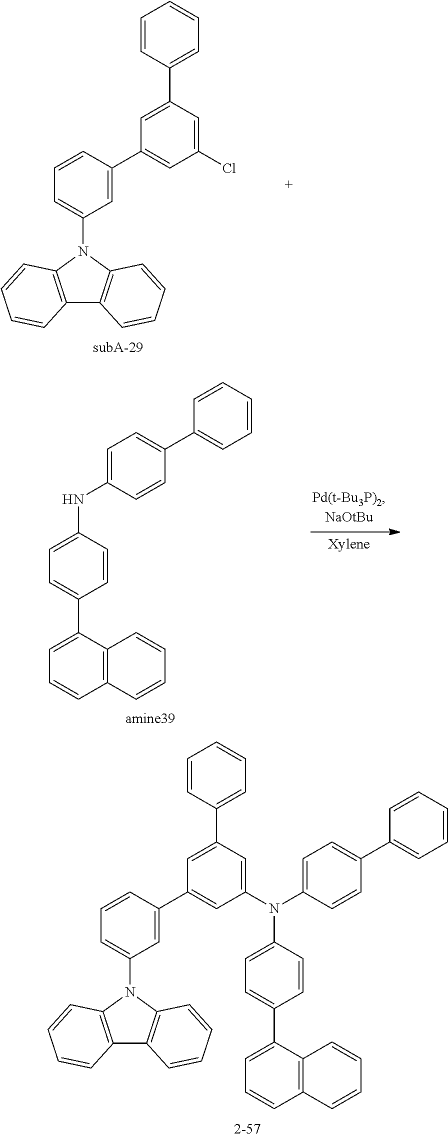 [SMILES: Clc1cc(-c2ccccc2)cc(-c2cccc(-n3c4ccccc4c4ccccc43)c2)c1 ; c1ccc(-c2ccc(Nc3ccc(-c4cccc5ccccc45)cc3)cc2)cc1 ; c1ccc(-c2ccc(N(c3ccc(-c4cccc5ccccc45)cc3)c3cc(-c4ccccc4)cc(-c4cccc(-n5c6ccccc6c6ccccc65)c4)c3)cc2)cc1]

Compound subA-29 (10 g, 23.3 mmol), Compound amine39 (9.1 g, 24.4 mmol) and sodium tert-butoxide (2.9 g, 30.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13 g of Compound 2-57. (Yield: 73%, MS: [M+H]⁺=765)

### Synthesis Example 2-58

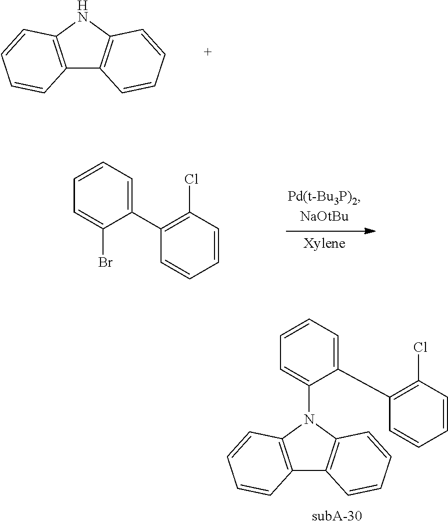 [SMILES: c1ccc2c(c1)[nH]c1ccccc12 ; Clc1ccccc1-c1ccccc1Br ; Clc1ccccc1-c1ccccc1-n1c2ccccc2c2ccccc21]

9H-carbazole (10 g, 59.8 mmol), 2-bromo-2′-chloro-1,1′-biphenyl (16.8 g, 62.8 mmol) and sodium tert-butoxide (7.5 g, 77.7 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 15 g of Compound subA-30. (Yield: 71%, MS: [M+H]⁺=354)

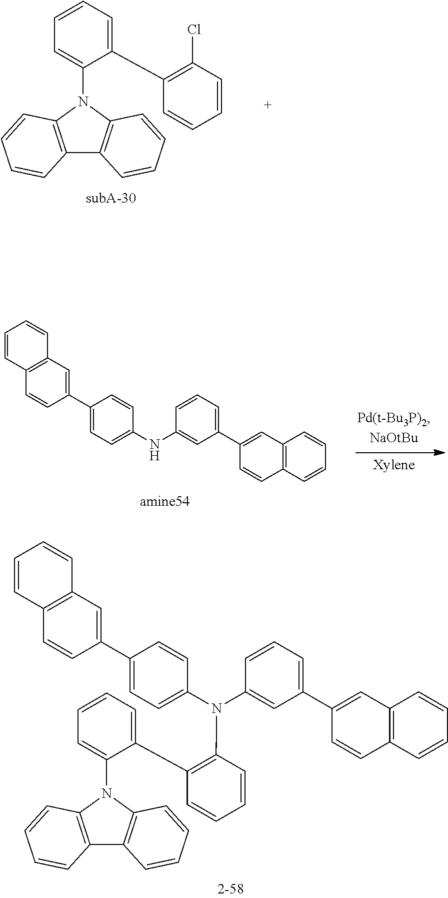 [SMILES: Clc1ccccc1-c1ccccc1-n1c2ccccc2c2ccccc21 ; c1cc(Nc2ccc(-c3ccc4ccccc4c3)cc2)cc(-c2ccc3ccccc3c2)c1 ; c1cc(-c2ccc3ccccc3c2)cc(N(c2ccc(-c3ccc4ccccc4c3)cc2)c2ccccc2-c2ccccc2-n2c3ccccc3c3ccccc32)c1]

Compound subA-30 (10 g, 28.3 mmol), Compound amine54 (12.5 g, 29.7 mmol) and sodium tert-butoxide (3.5 g, 36.7 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 12.7 g of Compound 2-58. (Yield: 61%, MS: [M+H]⁺=739)

### Synthesis Example 2-59

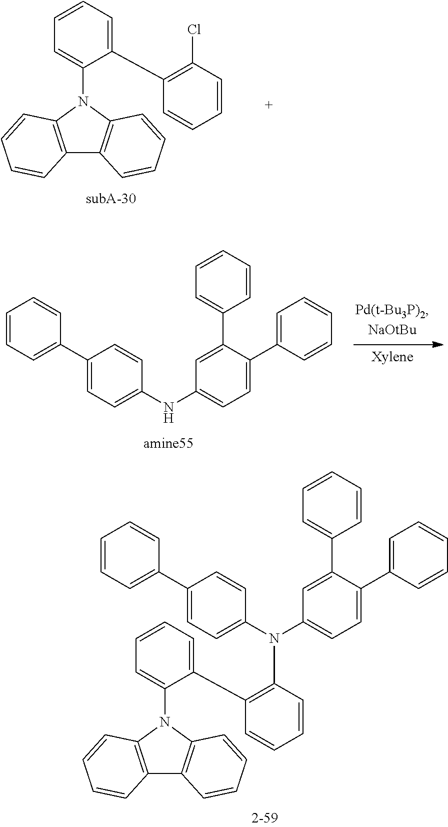 [SMILES: Clc1ccccc1-c1ccccc1-n1c2ccccc2c2ccccc21 ; c1ccc(-c2ccc(Nc3ccc(-c4ccccc4)c(-c4ccccc4)c3)cc2)cc1 ; c1ccc(-c2ccc(N(c3ccc(-c4ccccc4)c(-c4ccccc4)c3)c3ccccc3-c3ccccc3-n3c4ccccc4c4ccccc43)cc2)cc1]

Compound subA-30 (10 g, 28.3 mmol), Compound amine55 (11.8 g, 29.7 mmol) and sodium tert-butoxide (3.5 g, 36.7 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.9 g of Compound 2-59. (Yield: 69%, MS: [M+H]⁺=715)

### Synthesis Example 2-60

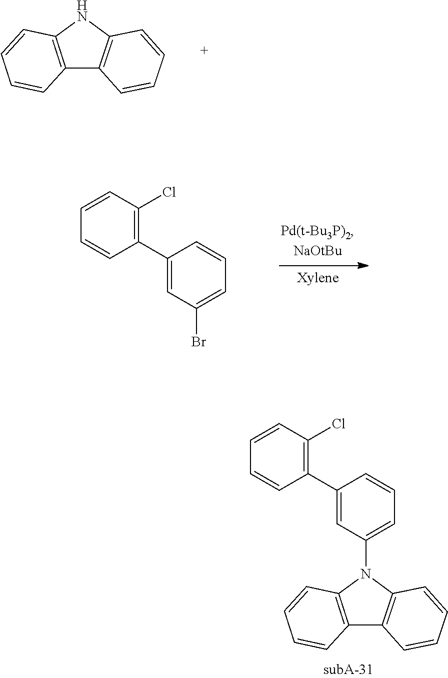 [SMILES: c1ccc2c(c1)[nH]c1ccccc12 ; Clc1ccccc1-c1cccc(Br)c1 ; Clc1ccccc1-c1cccc(-n2c3ccccc3c3ccccc32)c1]

9H-carbazole (10 g, 59.8 mmol), 3′-bromo-2-chloro-1,1′-biphenyl (16.8 g, 62.8 mmol) and sodium tert-butoxide (7.5 g, 77.7 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.1 g of Compound subA-31. (Yield: 62%, MS: [M+H]⁺=354)

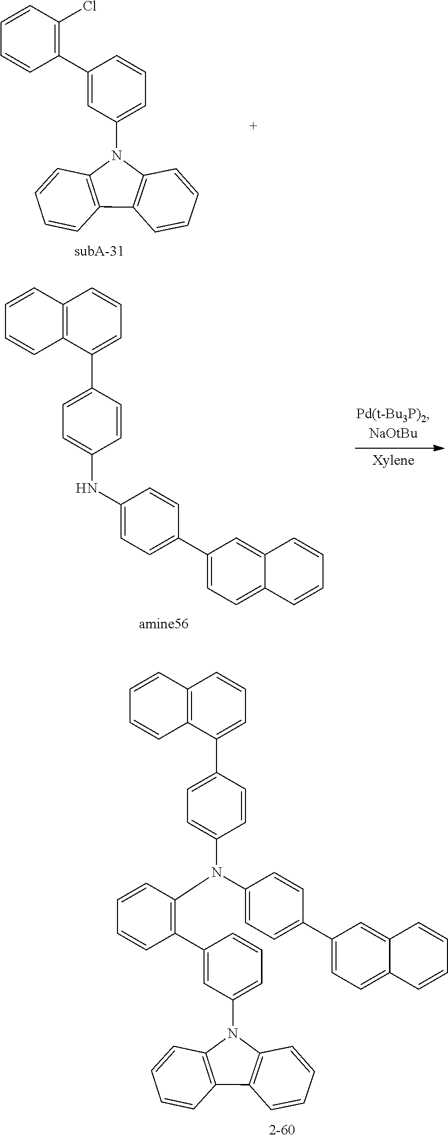 [SMILES: Clc1ccccc1-c1cccc(-n2c3ccccc3c3ccccc32)c1 ; c1ccc2cc(-c3ccc(Nc4ccc(-c5cccc6ccccc56)cc4)cc3)ccc2c1 ; c1cc(-c2ccccc2N(c2ccc(-c3ccc4ccccc4c3)cc2)c2ccc(-c3cccc4ccccc34)cc2)cc(-n2c3ccccc3c3ccccc32)c1]

Compound subA-31 (10 g, 28.3 mmol), Compound amine56 (12.5 g, 29.7 mmol) and sodium tert-butoxide (3.5 g, 36.7 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 14.8 g of Compound 2-60. (Yield: 71%, MS: [M+H]⁺=739)

### Synthesis Example 2-61

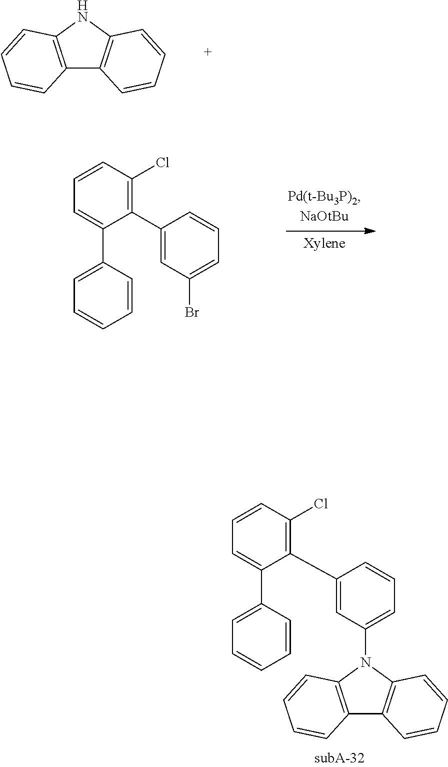 [SMILES: c1ccc2c(c1)[nH]c1ccccc12 ; Clc1cccc(-c2ccccc2)c1-c1cccc(Br)c1 ; Clc1cccc(-c2ccccc2)c1-c1cccc(-n2c3ccccc3c3ccccc32)c1]

9H-carbazole (10 g, 59.8 mmol), 3-bromo-6′-chloro-1,1′:2′,1″-terphenyl (21.6 g, 62.8 mmol) and sodium tert-butoxide (7.5 g, 77.7 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 17.5 g of Compound subA-32. (Yield: 68%, MS: [M+H]⁺=430)

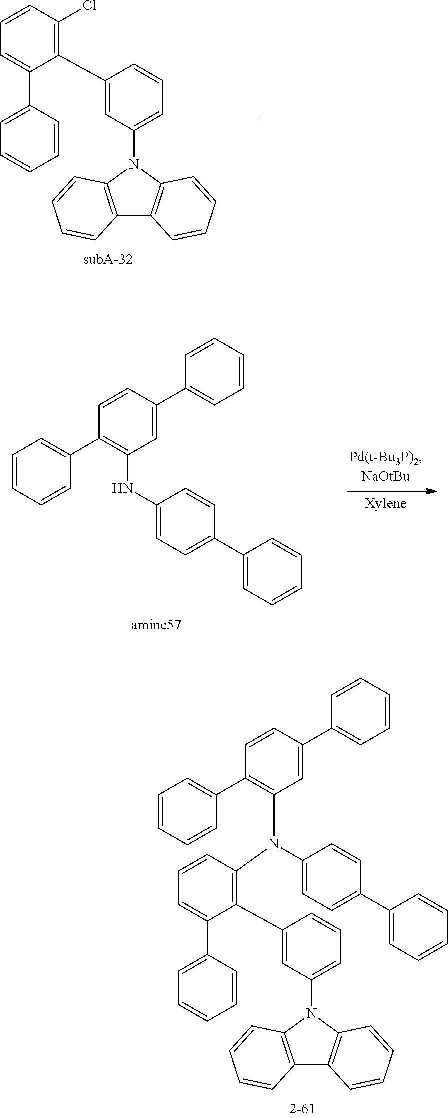 [SMILES: Clc1cccc(-c2ccccc2)c1-c1cccc(-n2c3ccccc3c3ccccc32)c1 ; c1ccc(-c2ccc(Nc3cc(-c4ccccc4)ccc3-c3ccccc3)cc2)cc1 ; c1ccc(-c2ccc(N(c3cc(-c4ccccc4)ccc3-c3ccccc3)c3cccc(-c4ccccc4)c3-c3cccc(-n4c5ccccc5c5ccccc54)c3)cc2)cc1]

Compound subA-32 (10 g, 23.3 mmol), Compound amine57 (9.7 g, 24.4 mmol) and sodium tert-butoxide (2.9 g, 30.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.4 g of Compound 2-61. (Yield: 73%, MS: [M+H]⁺=791)

### Synthesis Example 2-62

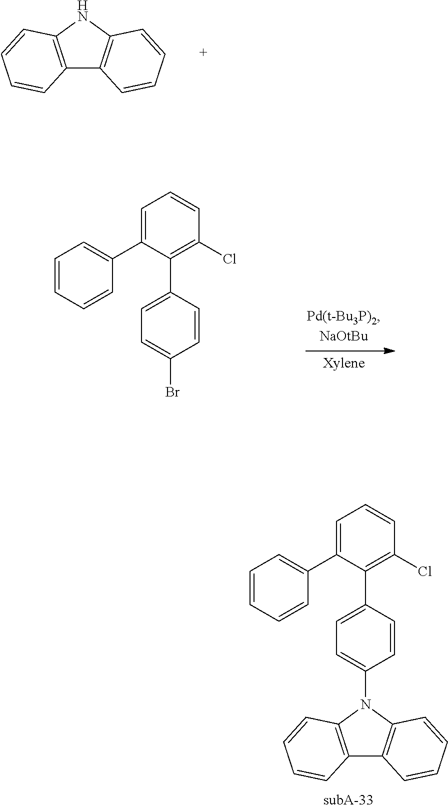 [SMILES: c1ccc2c(c1)[nH]c1ccccc12 ; Clc1cccc(-c2ccccc2)c1-c1ccc(Br)cc1 ; Clc1cccc(-c2ccccc2)c1-c1ccc(-n2c3ccccc3c3ccccc32)cc1]

9H-carbazole (10 g, 59.8 mmol), 4″-bromo-3′-chloro-1,1′:2′,1″-terphenyl (21.6 g, 62.8 mmol) and sodium tert-butoxide (7.5 g, 77.7 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 16.9 g of Compound subA-33. (Yield: 66%, MS: [M+H]⁺=430)

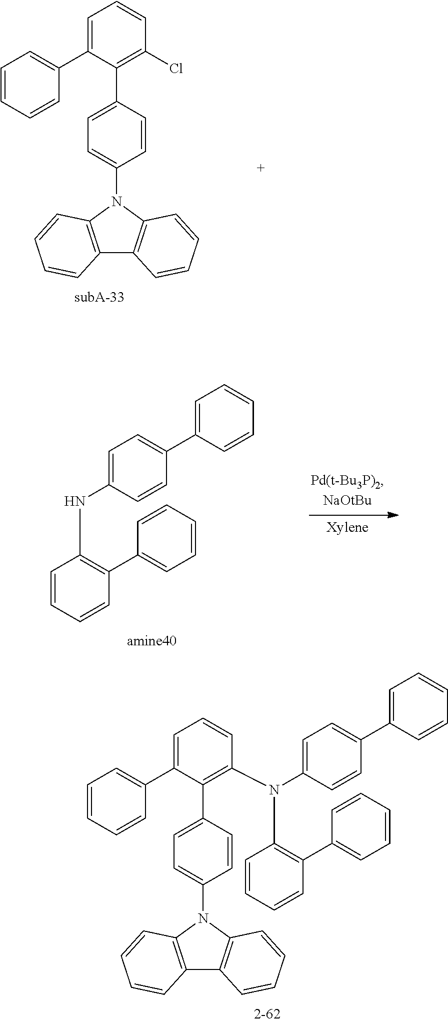 [SMILES: Clc1cccc(-c2ccccc2)c1-c1ccc(-n2c3ccccc3c3ccccc32)cc1 ; c1ccc(-c2ccc(Nc3ccccc3-c3ccccc3)cc2)cc1 ; c1ccc(-c2ccc(N(c3ccccc3-c3ccccc3)c3cccc(-c4ccccc4)c3-c3ccc(-n4c5ccccc5c5ccccc54)cc3)cc2)cc1]

Compound subA-33 (10 g, 23.3 mmol) and Compound amine40 (7.8 g, 24.4 mmol), sodium tert-butoxide (2.9 g, 30.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 10.1 g of Compound 2-62. (Yield: 61%, MS: [M+H]⁺=715)

### Synthesis Example 2-63

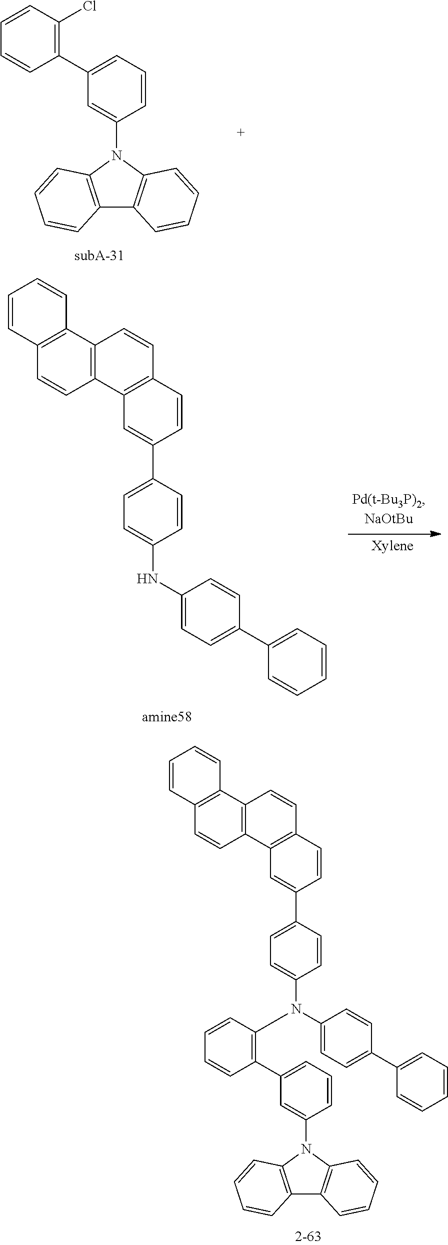 [SMILES: Clc1ccccc1-c1cccc(-n2c3ccccc3c3ccccc32)c1 ; c1ccc(-c2ccc(Nc3ccc(-c4ccc5ccc6c7ccccc7ccc6c5c4)cc3)cc2)cc1 ; c1ccc(-c2ccc(N(c3ccc(-c4ccc5ccc6c7ccccc7ccc6c5c4)cc3)c3ccccc3-c3cccc(-n4c5ccccc5c5ccccc54)c3)cc2)cc1]

Compound subA-31 (10 g, 28.3 mmol), Compound amine58 (14 g, 29.7 mmol) and sodium tert-butoxide (3.5 g, 36.7 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 14.5 g of Compound 2-63. (Yield: 65%, MS: [M+H]⁺=789)

### Synthesis Example 2-64

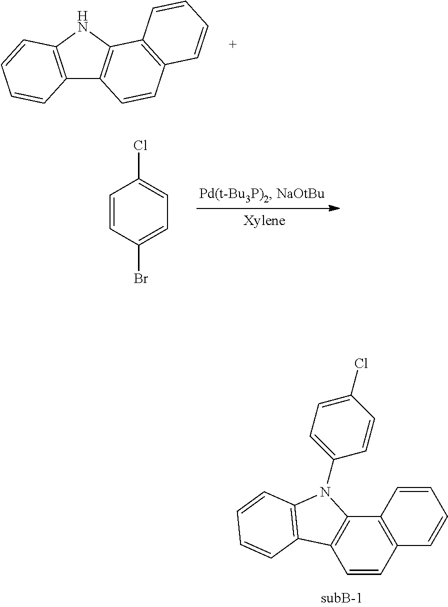

11H-benzo[a]carbazole (10 g, 46 mmol), 1-bromo-4-chlorobenzene (9.3 g, 48.3 mmol) and sodium tert-butoxide (5.7 g, 59.8 mmol) were added 10 to 200 ml of xylene under a nitrogen atmosphere, and the mixture was stirred and refluxed. Then, bis(tri-tert-butylphosphine)palladium(0) (0.2 g, 0.5 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 9.5 g of Compound subB-1. (Yield: 63%, MS: [M+H]⁺=328)

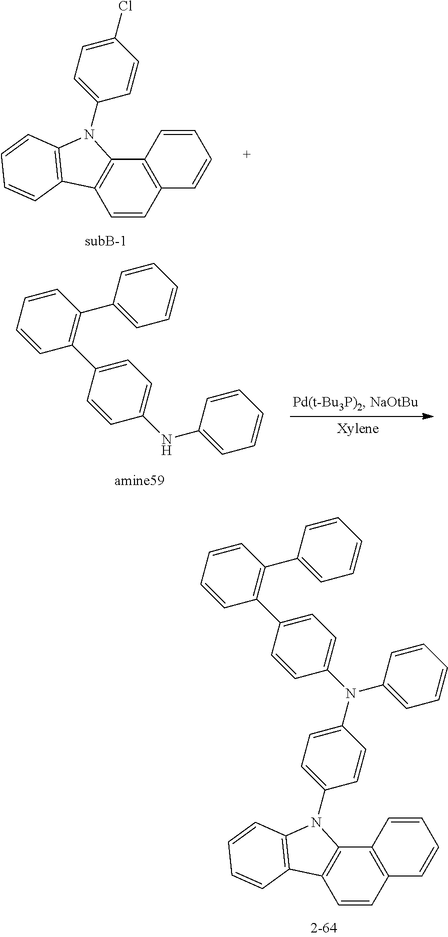 [SMILES: Clc1ccc(-n2c3ccccc3c3ccc4ccccc4c32)cc1 ; c1ccc(Nc2ccc(-c3ccccc3-c3ccccc3)cc2)cc1 ; c1ccc(-c2ccccc2-c2ccc(N(c3ccccc3)c3ccc(-n4c5ccccc5c5ccc6ccccc6c54)cc3)cc2)cc1]

Compound subB-1 (10 g, 30.5 mmol), Compound amine59 (10.3 g, 32 mmol) and sodium tert-butoxide (3.8 g, 39.7 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.1 g of Compound 2-64. (Yield: 70%, MS: [M+H]⁺=613)

### Synthesis Example 2-65

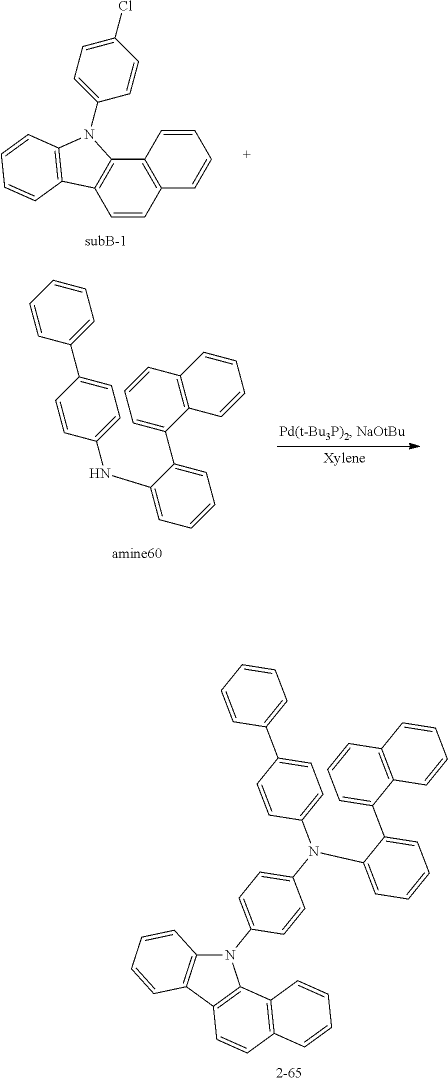 [SMILES: Clc1ccc(-n2c3ccccc3c3ccc4ccccc4c32)cc1 ; c1ccc(-c2ccc(Nc3ccccc3-c3cccc4ccccc34)cc2)cc1 ; c1ccc(-c2ccc(N(c3ccc(-n4c5ccccc5c5ccc6ccccc6c54)cc3)c3ccccc3-c3cccc4ccccc34)cc2)cc1]

Compound subB-1 (10 g, 30.5 mmol), Compound amine60 (11.9 g, 32 mmol) and sodium tert-butoxide (3.8 g, 39.7 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 12.5 g of Compound 2-65. (Yield: 62%, MS: [M+H]⁺=663)

### Synthesis Example 2-66

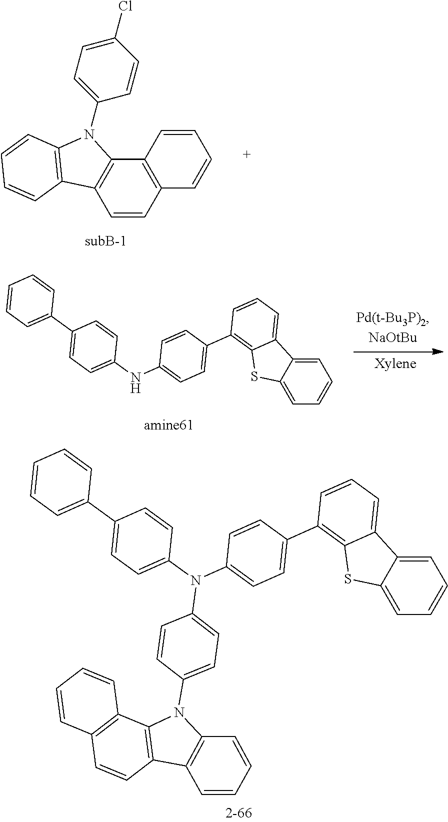 [SMILES: Clc1ccc(-n2c3ccccc3c3ccc4ccccc4c32)cc1 ; c1ccc(-c2ccc(Nc3ccc(-c4cccc5c4sc4ccccc45)cc3)cc2)cc1 ; c1ccc(-c2ccc(N(c3ccc(-c4cccc5c4sc4ccccc45)cc3)c3ccc(-n4c5ccccc5c5ccc6ccccc6c54)cc3)cc2)cc1]

Compound subB-1 (10 g, 30.5 mmol), Compound amine61 (23.3 g, 32 mmol) and sodium tert-butoxide (3.8 g, 39.7 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 14.2 g of Compound 2-66. (Yield: 65%, MS: [M+H]⁺=719)

### Synthesis Example 2-67

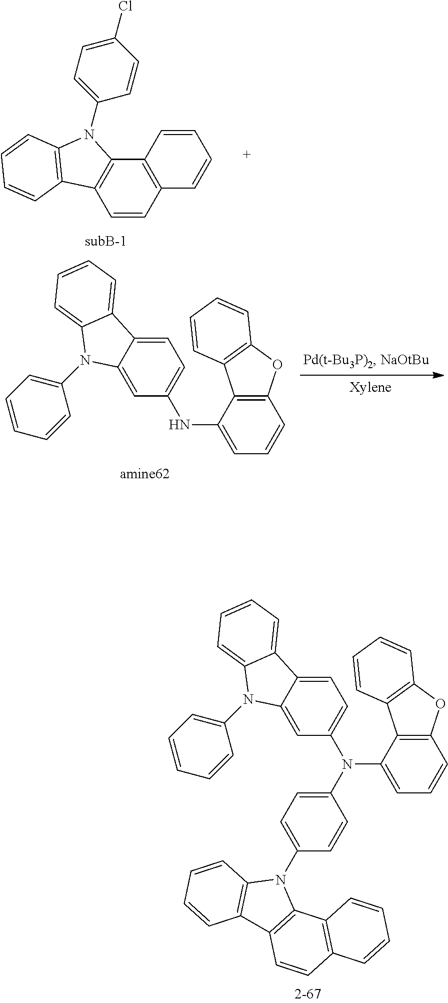 [SMILES: Clc1ccc(-n2c3ccccc3c3ccc4ccccc4c32)cc1 ; c1ccc(-n2c3ccccc3c3ccc(Nc4cccc5oc6ccccc6c45)cc32)cc1 ; c1ccc(-n2c3ccccc3c3ccc(N(c4ccc(-n5c6ccccc6c6ccc7ccccc7c65)cc4)c4cccc5oc6ccccc6c45)cc32)cc1]

Compound subB-1 (10 g, 30.5 mmol), Compound amine62 (13.6 g, 32 mmol) and sodium tert-butoxide (3.8 g, 39.7 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 16.4 g of Compound 2-67. (Yield: 75%, MS: [M+H]⁺=716)

### Synthesis Example 2-68

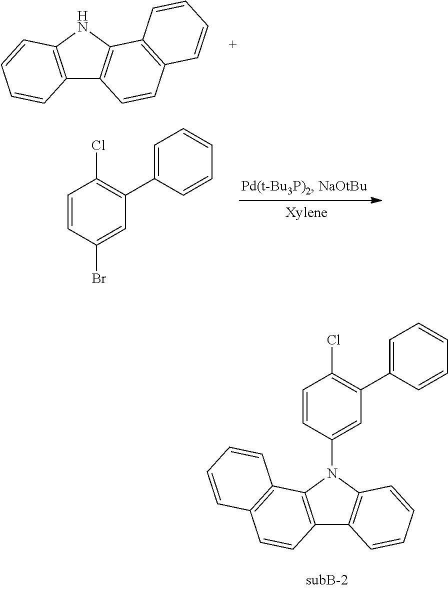

11H-benzo[a]carbazole (10 g, 46 mmol), 5-bromo-2-chloro-1,1′-biphenyl (12.9 g, 48.3 mmol) and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.2 g, 0.5 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.9 g of Compound subB-2. (Yield: 75%, MS: [M+H]⁺=404)

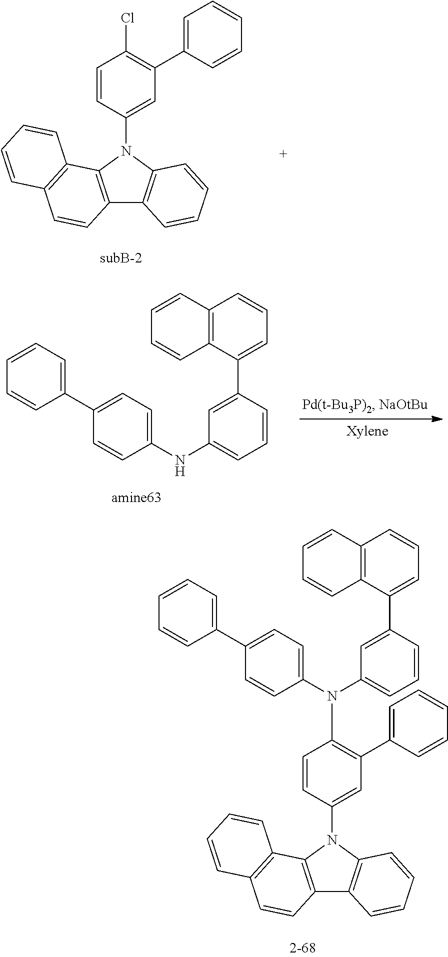 [SMILES: Clc1ccc(-n2c3ccccc3c3ccc4ccccc4c32)cc1-c1ccccc1 ; c1ccc(-c2ccc(Nc3cccc(-c4cccc5ccccc45)c3)cc2)cc1 ; c1ccc(-c2ccc(N(c3cccc(-c4cccc5ccccc45)c3)c3ccc(-n4c5ccccc5c5ccc6ccccc6c54)cc3-c3ccccc3)cc2)cc1]

Compound subB-2 (10 g, 24.8 mmol), Compound amine63 (9.7 g, 26 mmol) and sodium tert-butoxide (3.1 g, 32.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.2 g of Compound 2-68. (Yield: 72%, MS: [M+H]⁺=739)

### Synthesis Example 2-69

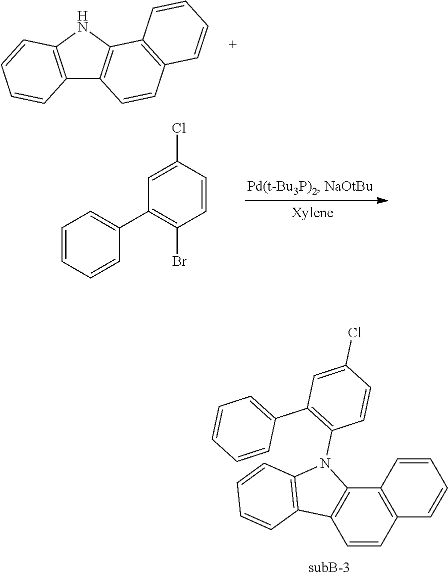

11H-benzo[a]carbazole (10 g, 46 mmol), 2-bromo-5-chloro-1,1′-biphenyl (12.9 g, 48.3 mmol) and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.2 g, 0.5 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 12.4 g of Compound subB-3. (Yield: 67%, MS: [M+H]⁺=404)

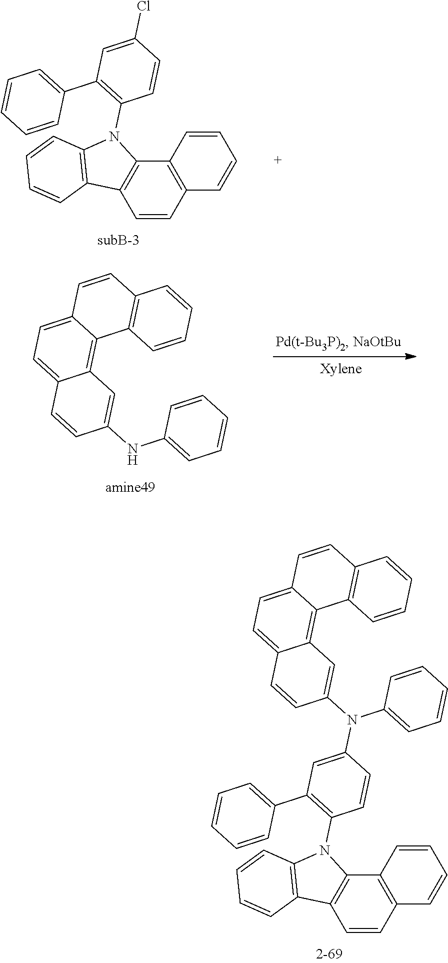 [SMILES: Clc1ccc(-n2c3ccccc3c3ccc4ccccc4c32)c(-c2ccccc2)c1 ; c1ccc(Nc2ccc3ccc4ccc5ccccc5c4c3c2)cc1 ; c1ccc(-c2cc(N(c3ccccc3)c3ccc4ccc5ccc6ccccc6c5c4c3)ccc2-n2c3ccccc3c3ccc4ccccc4c32)cc1]

Compound subB-3 (10 g, 24.8 mmol), Compound amine49 (8.3 g, 26 mmol) and sodium tert-butoxide (3.1 g, 32.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 11.6 g of Compound 2-69. (Yield: 68%, MS: [M+H]⁺=687)

### Synthesis Example 2-70

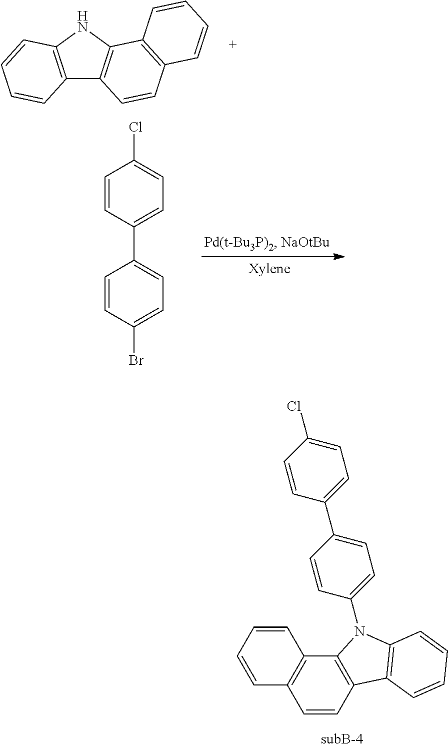

11H-benzo[a]carbazole (10 g, 46 mmol) and 4-bromo-4′-chloro-1,1′-biphenyl (12.9 g, 48.3 mmol), sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.2 g, 0.5 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 12.1 g of Compound subB-4. (Yield: 65%, MS: [M+H]⁺=404)

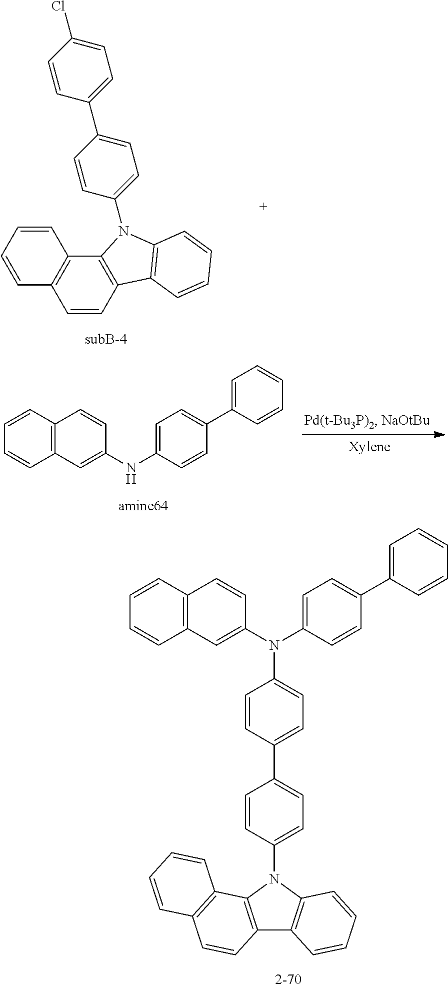 [SMILES: Clc1ccc(-c2ccc(-n3c4ccccc4c4ccc5ccccc5c43)cc2)cc1 ; c1ccc(-c2ccc(Nc3ccc4ccccc4c3)cc2)cc1 ; c1ccc(-c2ccc(N(c3ccc(-c4ccc(-n5c6ccccc6c6ccc7ccccc7c65)cc4)cc3)c3ccc4ccccc4c3)cc2)cc1]

Compound subB-4 (10 g, 24.8 mmol), Compound amine64 (7.7 g, 26 mmol) and sodium tert-butoxide (3.1 g, 32.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 11.6 g of Compound 2-70. (Yield: 71%, MS: [M+H]⁺=663)

### Synthesis Example 2-71

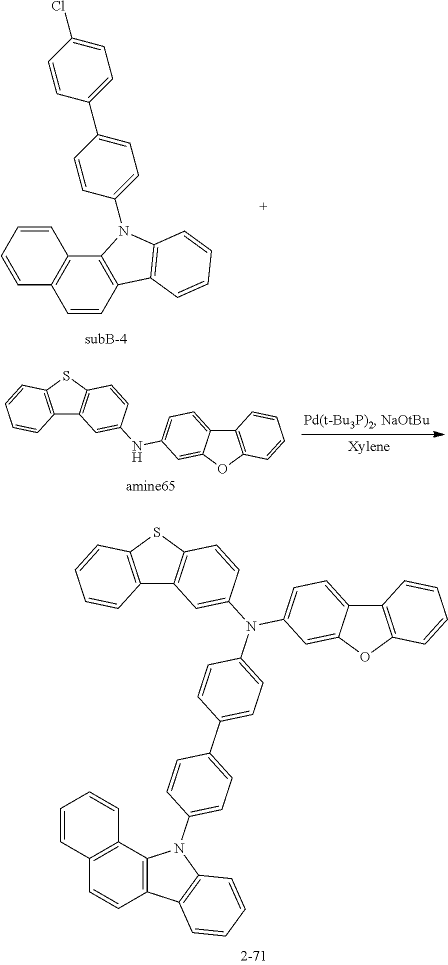 [SMILES: Clc1ccc(-c2ccc(-n3c4ccccc4c4ccc5ccccc5c43)cc2)cc1 ; c1ccc2c(c1)oc1cc(Nc3ccc4sc5ccccc5c4c3)ccc12 ; c1ccc2c(c1)ccc1c3ccccc3n(-c3ccc(-c4ccc(N(c5ccc6c(c5)oc5ccccc56)c5ccc6sc7ccccc7c6c5)cc4)cc3)c21]

Compound subB-4 (10 g, 24.8 mmol), Compound amine65 (7.7 g, 26 mmol) and sodium tert-butoxide (3.1 g, 32.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 11.6 g of Compound 2-71. (Yield: 71%, MS: [M+H]⁺=663)

### Synthesis Example 2-72

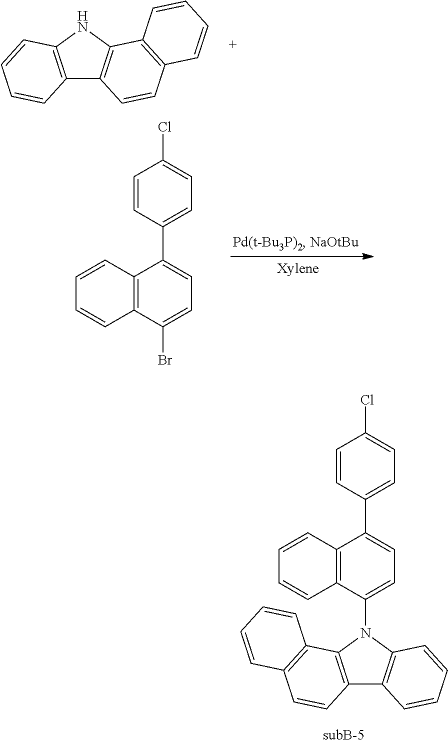

11H-benzo[a]carbazole (10 g, 46 mmol), 1-bromo-4-(4-chlorophenyl) naphthalene (15.3 g, 48.3 mmol) and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.2 g, 0.5 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 15.2 g of Compound subB-5. (Yield: 73%, MS: [M+H]⁺=454)

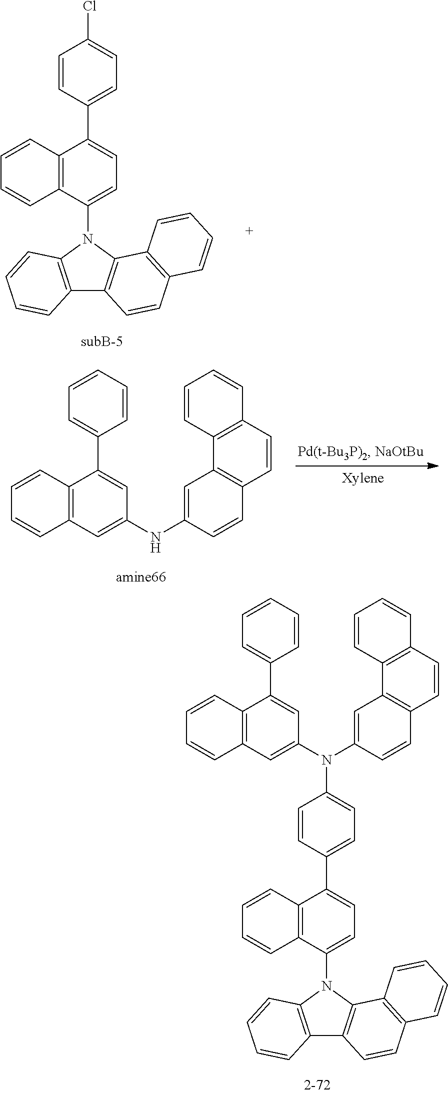 [SMILES: Clc1ccc(-c2ccc(-n3c4ccccc4c4ccc5ccccc5c43)c3ccccc23)cc1 ; c1ccc(-c2cc(Nc3ccc4ccc5ccccc5c4c3)cc3ccccc23)cc1 ; c1ccc(-c2cc(N(c3ccc(-c4ccc(-n5c6ccccc6c6ccc7ccccc7c65)c5ccccc45)cc3)c3ccc4ccc5ccccc5c4c3)cc3ccccc23)cc1]

Compound subB-5 (10 g, 22 mmol), Compound amine66 (9.1 g, 23.1 mmol) and sodium tert-butoxide (2.8 g, 28.6 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 12.2 g of Compound 2-72. (Yield: 68%, MS: [M+H]⁺=813)

### Synthesis Example 2-73

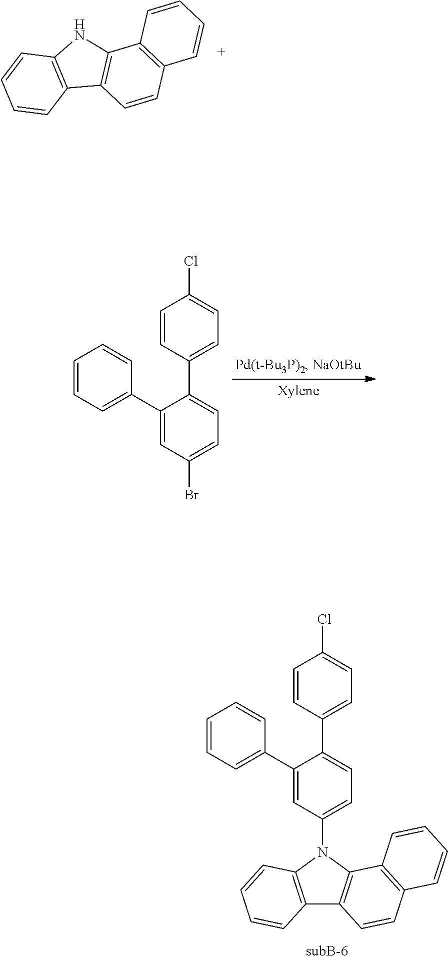 [SMILES: c1ccc2c(c1)ccc1c3ccccc3[nH]c21 ; Clc1ccc(-c2ccc(Br)cc2-c2ccccc2)cc1 ; Clc1ccc(-c2ccc(-n3c4ccccc4c4ccc5ccccc5c43)cc2-c2ccccc2)cc1]

11H-benzo[a]carbazole (10 g, 46 mmol), 4′-bromo-4-chloro-1,1′:2′,1″-terphenyl (16.6 g, 48.3 mmol) and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.2 g, 0.5 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 15 g of Compound subB-6. (Yield: 68%, MS: [M+H]⁺=480)

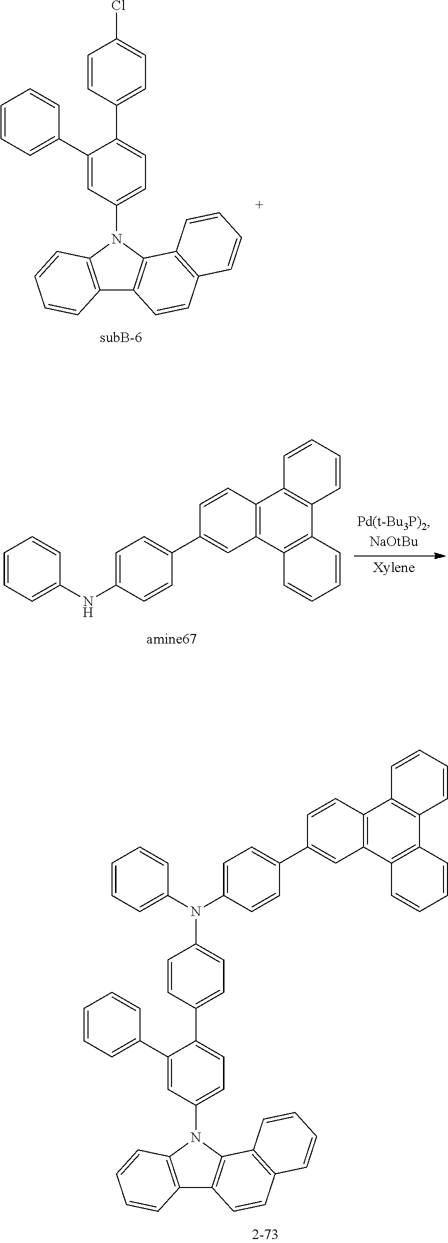 [SMILES: Clc1ccc(-c2ccc(-n3c4ccccc4c4ccc5ccccc5c43)cc2-c2ccccc2)cc1 ; c1ccc(Nc2ccc(-c3ccc4c5ccccc5c5ccccc5c4c3)cc2)cc1 ; c1ccc(-c2cc(-n3c4ccccc4c4ccc5ccccc5c43)ccc2-c2ccc(N(c3ccccc3)c3ccc(-c4ccc5c6ccccc6c6ccccc6c5c4)cc3)cc2)cc1]

Compound subB-6 (10 g, 20.8 mmol), Compound amine67 (8.7 g, 21.9 mmol) and sodium tert-butoxide (2.6 g, 27.1 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 12.4 g of Compound 2-73. (Yield: 71%, MS: [M+H]⁺=839)

### Synthesis Example 2-74

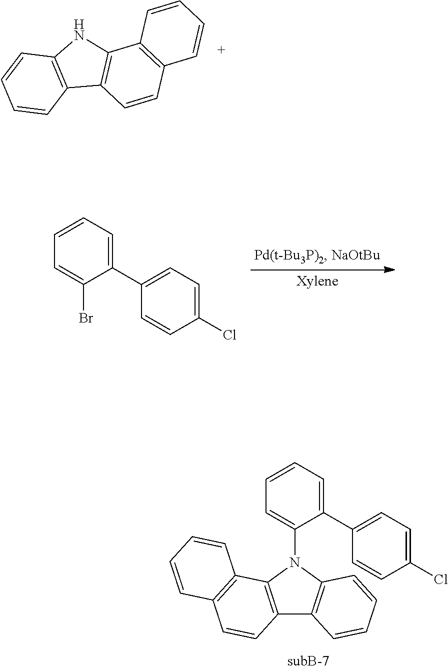 [SMILES: c1ccc2c(c1)ccc1c3ccccc3[nH]c21 ; Clc1ccc(-c2ccccc2Br)cc1 ; Clc1ccc(-c2ccccc2-n2c3ccccc3c3ccc4ccccc4c32)cc1]

11H-benzo[a]carbazole (10 g, 46 mmol), 2-bromo-4′-chloro-1,1′-biphenyl (12.9 g, 48.3 mmol) and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.2 g, 0.5 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.2 g of Compound subB-7. (Yield: 71%, MS: [M+H]⁺=404)

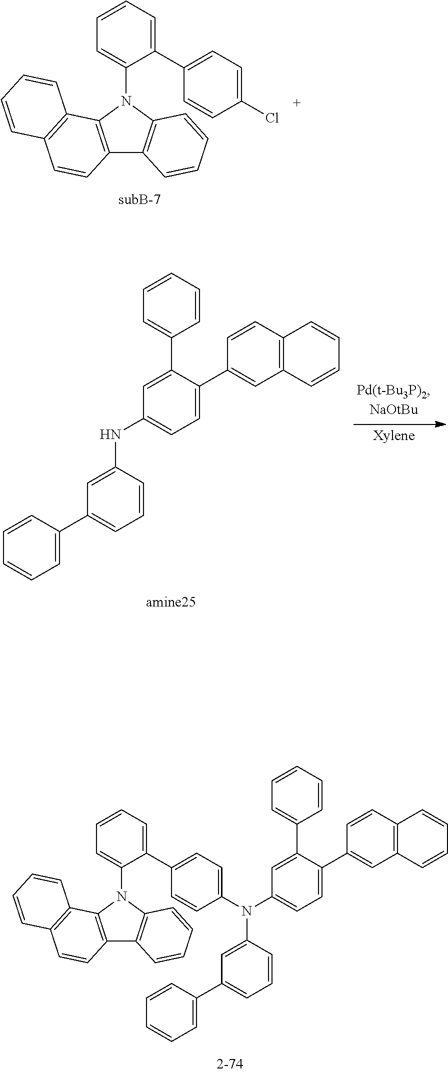 [SMILES: Clc1ccc(-c2ccccc2-n2c3ccccc3c3ccc4ccccc4c32)cc1 ; c1ccc(-c2cccc(Nc3ccc(-c4ccc5ccccc5c4)c(-c4ccccc4)c3)c2)cc1 ; c1ccc(-c2cccc(N(c3ccc(-c4ccccc4-n4c5ccccc5c5ccc6ccccc6c54)cc3)c3ccc(-c4ccc5ccccc5c4)c(-c4ccccc4)c3)c2)cc1]

Compound subB-7 (10 g, 24.8 mmol), Compound amine25 (11.6 g, 26 mmol) and sodium tert-butoxide (3.1 g, 32.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.3 g of Compound 2-74. (Yield: 66%, MS: [M+H]⁺=815)

### Synthesis Example 2-75

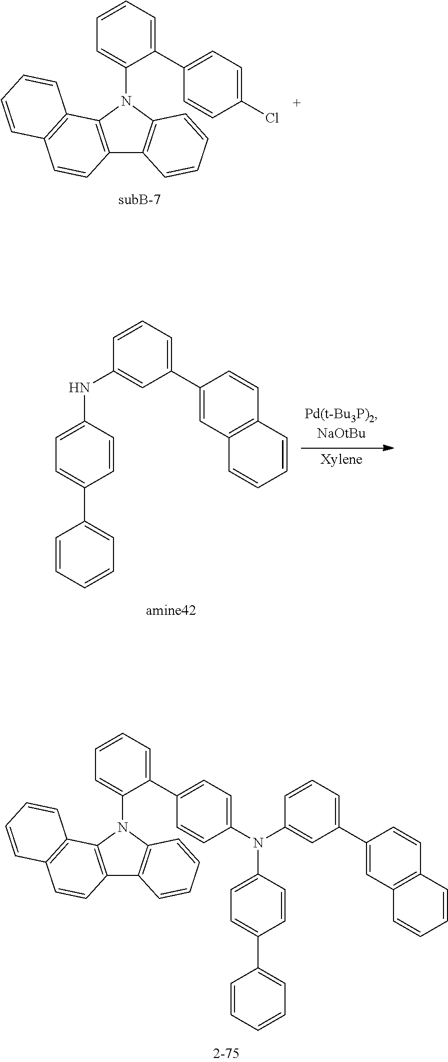 [SMILES: Clc1ccc(-c2ccccc2-n2c3ccccc3c3ccc4ccccc4c32)cc1 ; c1ccc(-c2ccc(Nc3cccc(-c4ccc5ccccc5c4)c3)cc2)cc1 ; c1ccc(-c2ccc(N(c3ccc(-c4ccccc4-n4c5ccccc5c5ccc6ccccc6c54)cc3)c3cccc(-c4ccc5ccccc5c4)c3)cc2)cc1]

Compound subB-7 (10 g, 24.8 mmol), Compound amine42 (9.7 g, 26 mmol) and sodium tert-butoxide (3.1 g, 32.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 12.2 g of Compound 2-75. (Yield: 67%, MS: [M+H]⁺=739)

### Synthesis Example 2-76

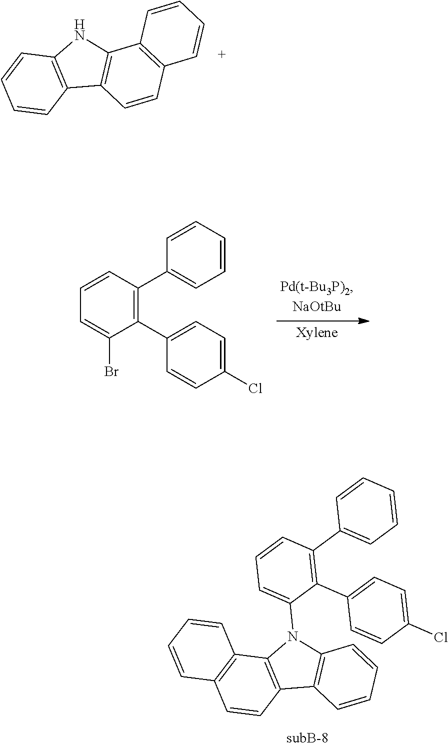 [SMILES: c1ccc2c(c1)ccc1c3ccccc3[nH]c21 ; Clc1ccc(-c2c(Br)cccc2-c2ccccc2)cc1 ; Clc1ccc(-c2c(-c3ccccc3)cccc2-n2c3ccccc3c3ccc4ccccc4c32)cc1]

11H-benzo[a]carbazole (10 g, 46 mmol), 3′-bromo-4″-chloro-1,1′:2′,1″-terphenyl (16.6 g, 48.3 mmol) and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.2 g, 0.5 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 14.3 g of Compound subB-8. (Yield: 65%, MS: [M+H]⁺=480)

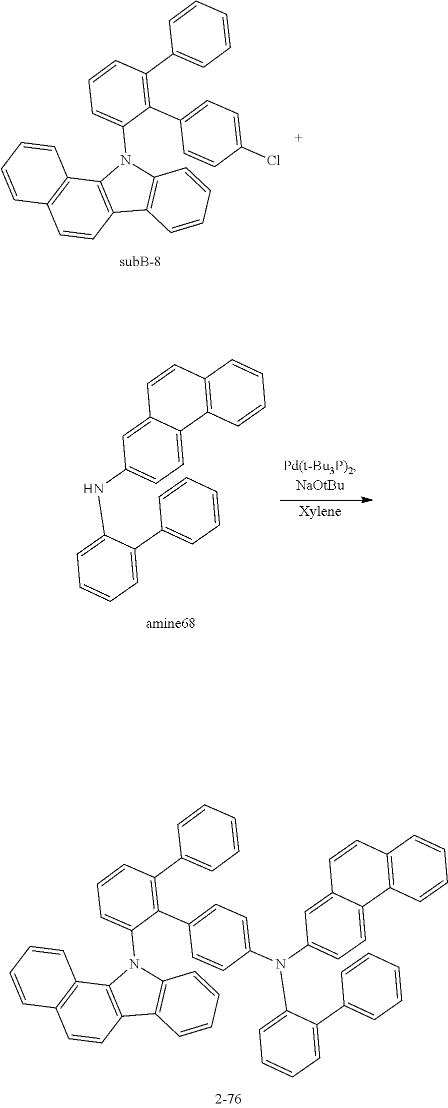 [SMILES: Clc1ccc(-c2c(-c3ccccc3)cccc2-n2c3ccccc3c3ccc4ccccc4c32)cc1 ; c1ccc(-c2ccccc2Nc2ccc3c(ccc4ccccc43)c2)cc1 ; c1ccc(-c2ccccc2N(c2ccc(-c3c(-c4ccccc4)cccc3-n3c4ccccc4c4ccc5ccccc5c43)cc2)c2ccc3c(ccc4ccccc43)c2)cc1]

Compound subB-8 (10 g, 20.8 mmol), Compound amine68 (7.6 g, 21.9 mmol) and sodium tert-butoxide (2.6 g, 27.1 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 12 g of Compound 2-76. (Yield: 73%, MS: [M+H]⁺=789)

### Synthesis Example 2-77

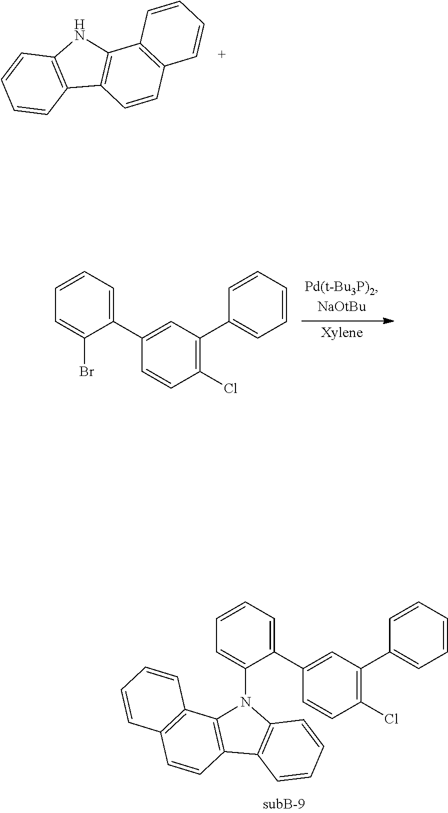 [SMILES: c1ccc2c(c1)ccc1c3ccccc3[nH]c21 ; Clc1ccc(-c2ccccc2Br)cc1-c1ccccc1 ; Clc1ccc(-c2ccccc2-n2c3ccccc3c3ccc4ccccc4c32)cc1-c1ccccc1]

11H-benzo[a]carbazole (10 g, 46 mmol), 2-bromo-4′-chloro-1,1′:3′,1″-terphenyl (16.6 g, 48.3 mmol) and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.2 g, 0.5 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 15.7 g of Compound subB-9. (Yield: 71%, MS: [M+H]⁺=480)

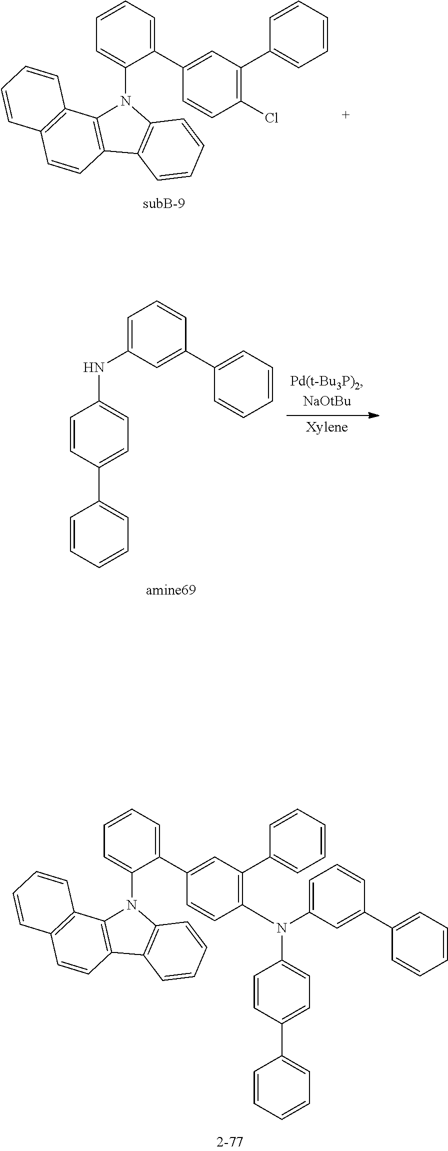 [SMILES: Clc1ccc(-c2ccccc2-n2c3ccccc3c3ccc4ccccc4c32)cc1-c1ccccc1 ; c1ccc(-c2ccc(Nc3cccc(-c4ccccc4)c3)cc2)cc1 ; c1ccc(-c2ccc(N(c3cccc(-c4ccccc4)c3)c3ccc(-c4ccccc4-n4c5ccccc5c5ccc6ccccc6c54)cc3-c3ccccc3)cc2)cc1]

Compound subB-9 (10 g, 20.8 mmol), Compound amine69 (7 g, 21.9 mmol) and sodium tert-butoxide (2.6 g, 27.1 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 9.6 g of Compound 2-77. (Yield: 60%, MS: [M+H]⁺=765)

### Synthesis Example 2-78

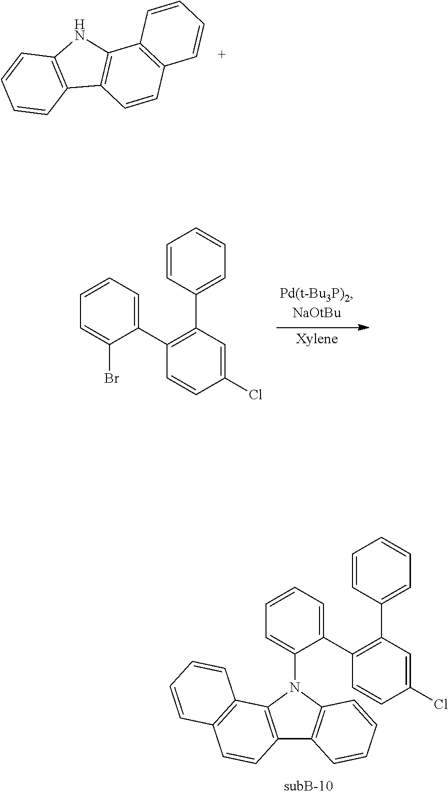 [SMILES: c1ccc2c(c1)ccc1c3ccccc3[nH]c21 ; Clc1ccc(-c2ccccc2Br)c(-c2ccccc2)c1 ; Clc1ccc(-c2ccccc2-n2c3ccccc3c3ccc4ccccc4c32)c(-c2ccccc2)c1]

11H-benzo[a]carbazole (10 g, 46 mmol), 2-bromo-4′-chloro-1,1′:2′,1″-terphenyl (16.6 g, 48.3 mmol) and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.2 g, 0.5 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 14.6 g of Compound subB-10. (Yield: 66%, MS: [M+H]⁺=480)

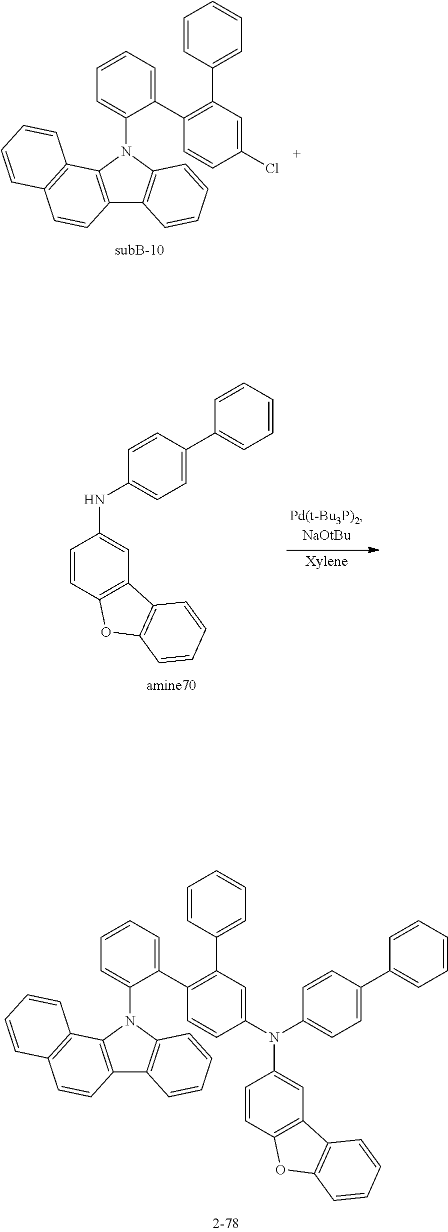 [SMILES: Clc1ccc(-c2ccccc2-n2c3ccccc3c3ccc4ccccc4c32)c(-c2ccccc2)c1 ; c1ccc(-c2ccc(Nc3ccc4oc5ccccc5c4c3)cc2)cc1 ; c1ccc(-c2ccc(N(c3ccc(-c4ccccc4-n4c5ccccc5c5ccc6ccccc6c54)c(-c4ccccc4)c3)c3ccc4oc5ccccc5c4c3)cc2)cc1]

Compound subB-10 (10 g, 20.8 mmol), Compound amine70 (7.3 g, 21.9 mmol) and sodium tert-butoxide (2.6 g, 27.1 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 11.8 g of Compound 2-78. (Yield: 73%, MS: [M+H]⁺=779)

### Synthesis Example 2-79

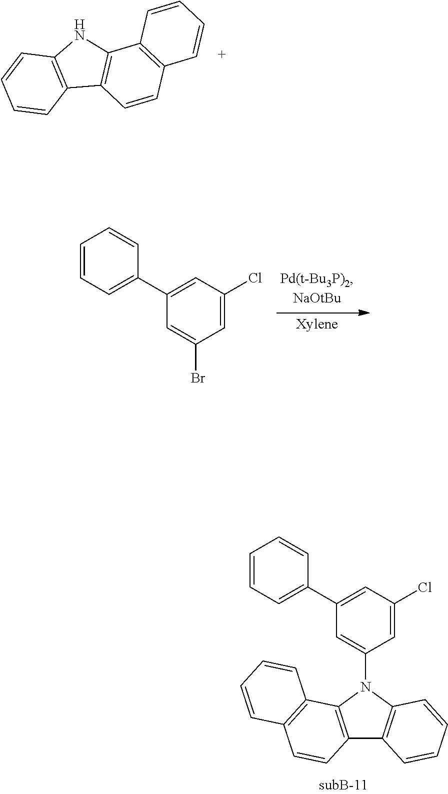 [SMILES: c1ccc2c(c1)ccc1c3ccccc3[nH]c21 ; Clc1cc(Br)cc(-c2ccccc2)c1 ; Clc1cc(-c2ccccc2)cc(-n2c3ccccc3c3ccc4ccccc4c32)c1]

11H-benzo[a]carbazole (10 g, 46 mmol), 3-bromo-5-chloro-1,1′-biphenyl (12.9 g, 48.3 mmol) and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.2 g, 0.5 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.5 g of Compound subB-11. (Yield: 73%, MS: [M+H]⁺=404)

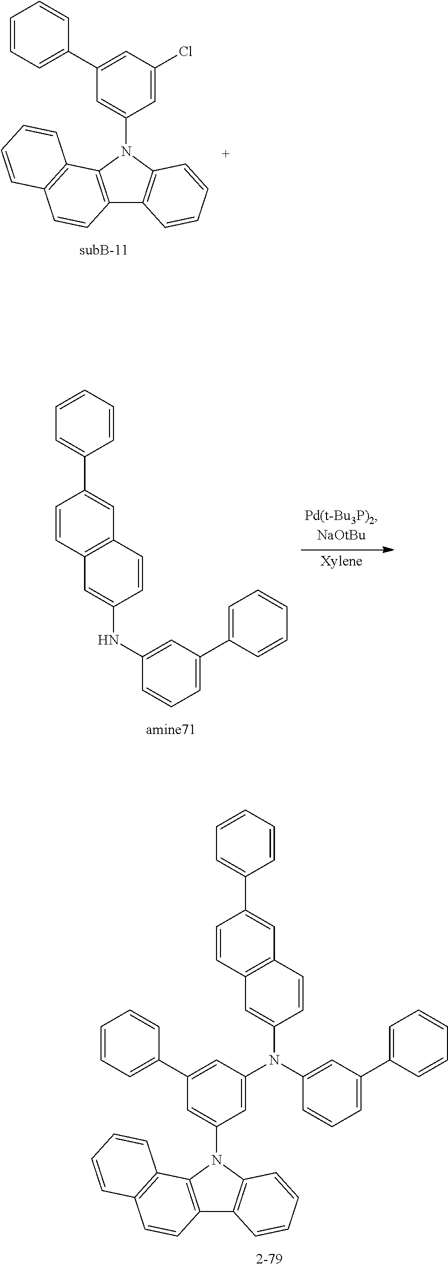 [SMILES: Clc1cc(-c2ccccc2)cc(-n2c3ccccc3c3ccc4ccccc4c32)c1 ; c1ccc(-c2cccc(Nc3ccc4cc(-c5ccccc5)ccc4c3)c2)cc1 ; c1ccc(-c2cccc(N(c3cc(-c4ccccc4)cc(-n4c5ccccc5c5ccc6ccccc6c54)c3)c3ccc4cc(-c5ccccc5)ccc4c3)c2)cc1]

Compound subB-11 (10 g, 24.8 mmol), Compound amine71 (9.7 g, 26 mmol) and sodium tert-butoxide (3.1 g, 32.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.3 g of Compound 2-79. (Yield: 73%, MS: [M+H]⁺=739)

### Synthesis Example 2-80

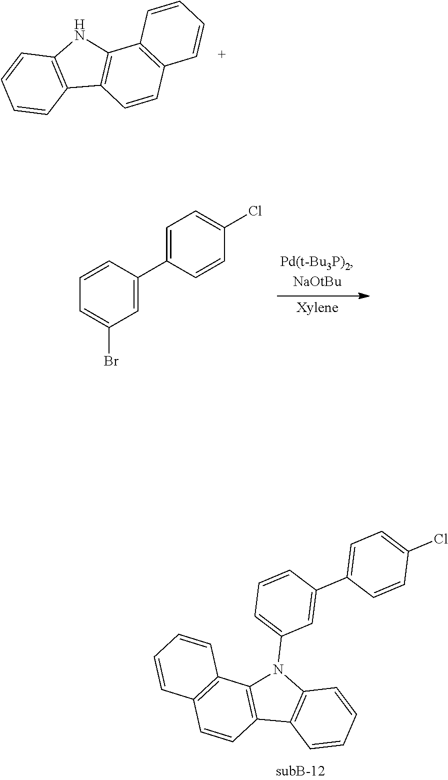 [SMILES: c1ccc2c(c1)ccc1c3ccccc3[nH]c21 ; Clc1ccc(-c2cccc(Br)c2)cc1 ; Clc1ccc(-c2cccc(-n3c4ccccc4c4ccc5ccccc5c43)c2)cc1]

11H-benzo[a]carbazole (10 g, 46 mmol), 3-bromo-4′-chloro-1,1′-biphenyl (12.9 g, 48.3 mmol) and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.2 g, 0.5 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.4 g of Compound subB-12. (Yield: 72%, MS: [M+H]⁺=404)

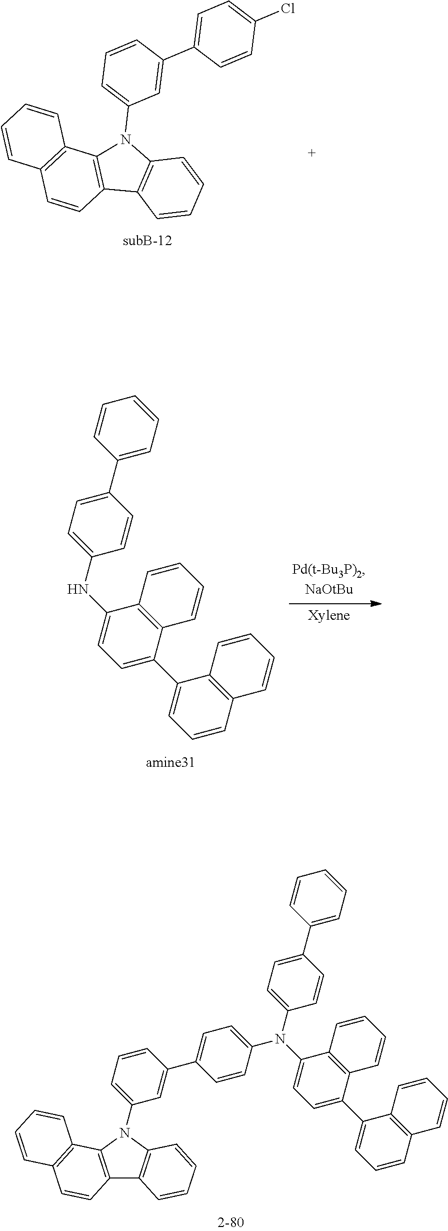 [SMILES: Clc1ccc(-c2cccc(-n3c4ccccc4c4ccc5ccccc5c43)c2)cc1 ; c1ccc(-c2ccc(Nc3ccc(-c4cccc5ccccc45)c4ccccc34)cc2)cc1 ; c1ccc(-c2ccc(N(c3ccc(-c4cccc(-n5c6ccccc6c6ccc7ccccc7c65)c4)cc3)c3ccc(-c4cccc5ccccc45)c4ccccc34)cc2)cc1]

Compound subB-12 (10 g, 24.8 mmol), Compound amine31 (11 g, 26 mmol) and sodium tert-butoxide (3.1 g, 32.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 12.1 g of Compound 2-80. (Yield: 62%, MS: [M+H]⁺=789)

### Synthesis Example 2-81

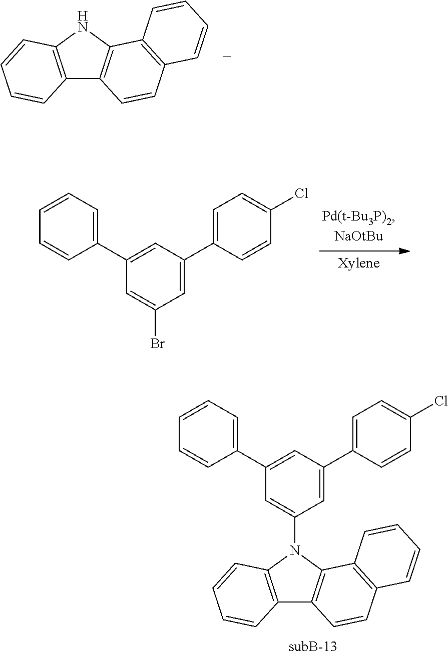 [SMILES: c1ccc2c(c1)ccc1c3ccccc3[nH]c21 ; Clc1ccc(-c2cc(Br)cc(-c3ccccc3)c2)cc1 ; Clc1ccc(-c2cc(-c3ccccc3)cc(-n3c4ccccc4c4ccc5ccccc5c43)c2)cc1]

11H-benzo[a]carbazole (10 g, 46 mmol), 5′-bromo-4-chloro-1,1′:3′,1″-terphenyl (16.6 g, 48.3 mmol) and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.2 g, 0.5 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 15.2 g of Compound subB-13. (Yield: 69%, MS: [M+H]⁺=480)

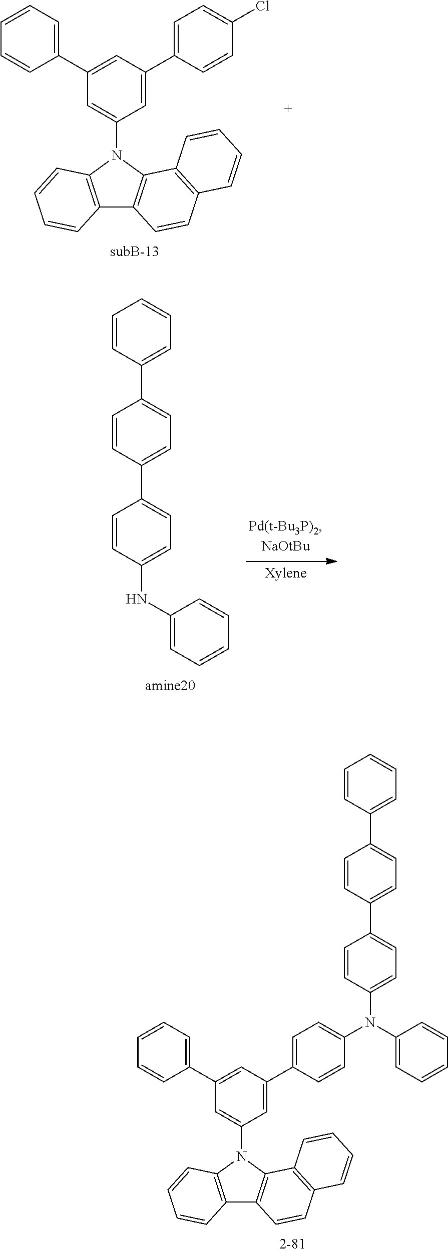 [SMILES: Clc1ccc(-c2cc(-c3ccccc3)cc(-n3c4ccccc4c4ccc5ccccc5c43)c2)cc1 ; c1ccc(Nc2ccc(-c3ccc(-c4ccccc4)cc3)cc2)cc1 ; c1ccc(-c2ccc(-c3ccc(N(c4ccccc4)c4ccc(-c5cc(-c6ccccc6)cc(-n6c7ccccc7c7ccc8ccccc8c76)c5)cc4)cc3)cc2)cc1]

Compound subB-13 (10 g, 20.8 mmol), Compound amine20 (7 g, 21.9 mmol) and sodium tert-butoxide (2.6 g, 27.1 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 11.1 g of Compound 2-81. (Yield: 70%, MS: [M+H]⁺=765)

### Synthesis Example 2-82

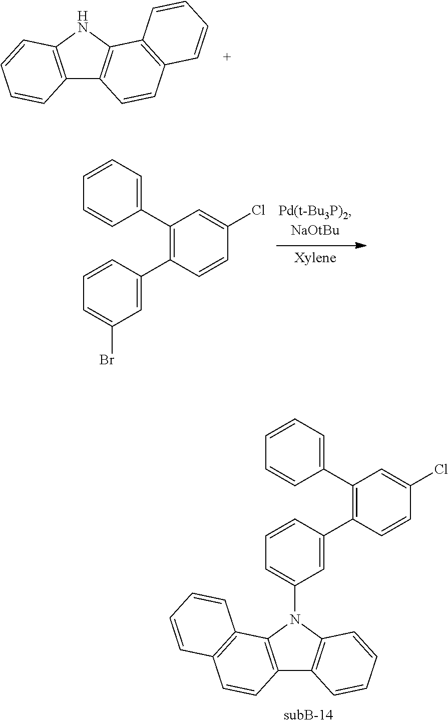 [SMILES: c1ccc2c(c1)ccc1c3ccccc3[nH]c21 ; Clc1ccc(-c2cccc(Br)c2)c(-c2ccccc2)c1 ; Clc1ccc(-c2cccc(-n3c4ccccc4c4ccc5ccccc5c43)c2)c(-c2ccccc2)c1]

11H-benzo[a]carbazole (10 g, 46 mmol), 3-bromo-4′-chloro-1,1′:2′,1″-terphenyl (16.6 g, 48.3 mmol) and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.2 g, 0.5 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.7 g of Compound subB-14. (Yield: 62%, MS: [M+H]⁺=480)

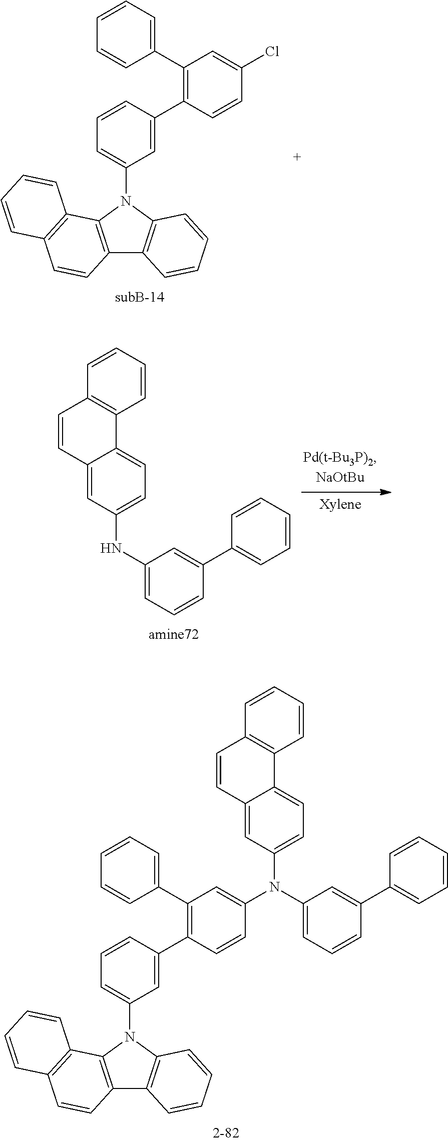 [SMILES: Clc1ccc(-c2cccc(-n3c4ccccc4c4ccc5ccccc5c43)c2)c(-c2ccccc2)c1 ; c1ccc(-c2cccc(Nc3ccc4c(ccc5ccccc54)c3)c2)cc1 ; c1ccc(-c2cccc(N(c3ccc(-c4cccc(-n5c6ccccc6c6ccc7ccccc7c65)c4)c(-c4ccccc4)c3)c3ccc4c(ccc5ccccc54)c3)c2)cc1]

Compound subB-14 (10 g, 20.8 mmol), Compound amine72 (7.6 g, 21.9 mmol) and sodium tert-butoxide (2.6 g, 27.1 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 11.5 g of Compound 2-82. (Yield: 70%, MS: [M+H]⁺=789)

### Synthesis Example 2-83

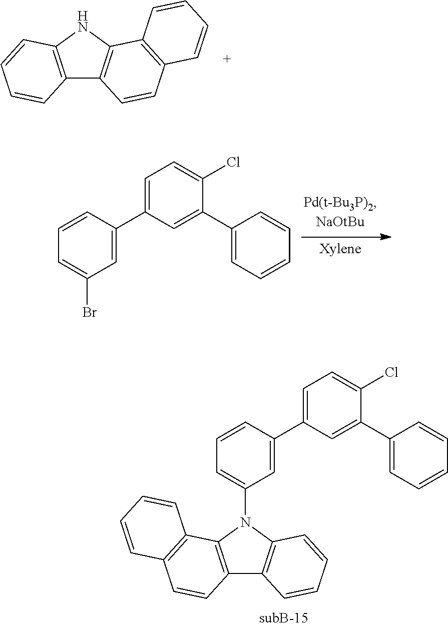 [SMILES: c1ccc2c(c1)ccc1c3ccccc3[nH]c21 ; Clc1ccc(-c2cccc(Br)c2)cc1-c1ccccc1 ; Clc1ccc(-c2cccc(-n3c4ccccc4c4ccc5ccccc5c43)c2)cc1-c1ccccc1]

11H-benzo[a]carbazole (10 g, 46 mmol), 3-bromo-4′-chloro-1,1′:3′,1″-terphenyl (16.6 g, 48.3 mmol) and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.2 g, 0.5 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 16.5 g of Compound subB-15. (Yield: 75%, MS: [M+H]⁺=480)

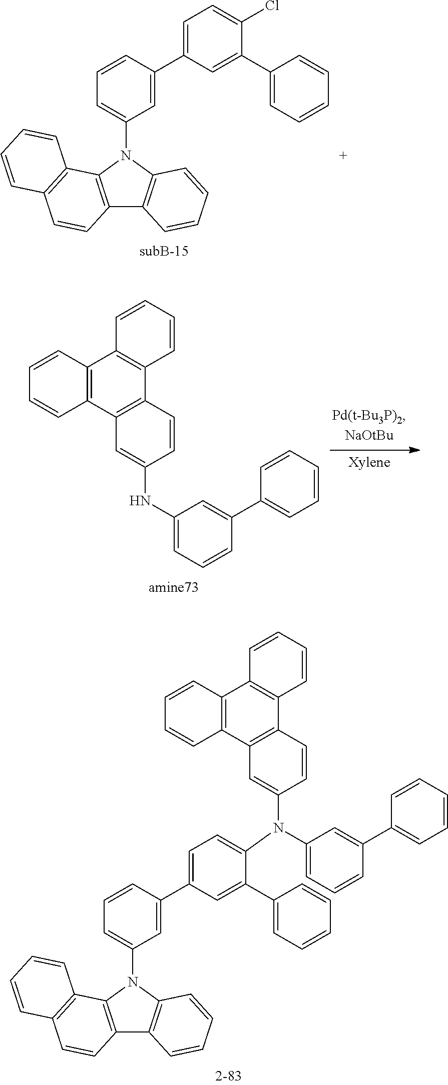 [SMILES: Clc1ccc(-c2cccc(-n3c4ccccc4c4ccc5ccccc5c43)c2)cc1-c1ccccc1 ; c1ccc(-c2cccc(Nc3ccc4c5ccccc5c5ccccc5c4c3)c2)cc1 ; c1ccc(-c2cccc(N(c3ccc4c5ccccc5c5ccccc5c4c3)c3ccc(-c4cccc(-n5c6ccccc6c6ccc7ccccc7c65)c4)cc3-c3ccccc3)c2)cc1]

Compound subB-15 (10 g, 20.8 mmol), Compound amine73 (8.7 g, 21.9 mmol) and sodium tert-butoxide (2.6 g, 27.1 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 12.1 g of Compound 2-83. (Yield: 69%, MS: [M+H]⁺=839)

### Synthesis Example 2-84

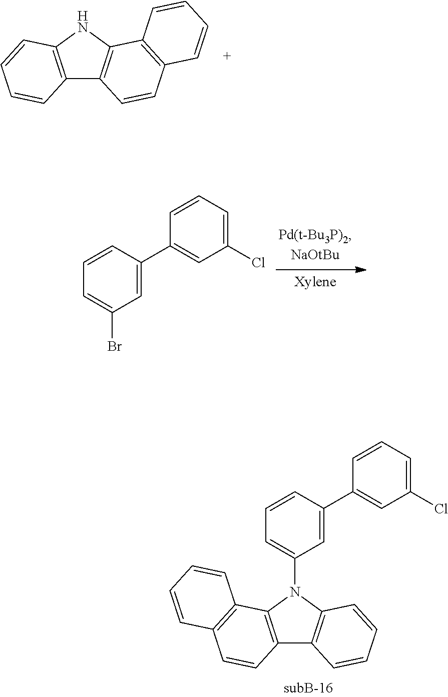 [SMILES: c1ccc2c(c1)ccc1c3ccccc3[nH]c21 ; Clc1cccc(-c2cccc(Br)c2)c1 ; Clc1cccc(-c2cccc(-n3c4ccccc4c4ccc5ccccc5c43)c2)c1]

11H-benzo[a]carbazole (10 g, 46 mmol), 3-bromo-3′-chloro-1,1′-biphenyl (12.9 g, 48.3 mmol) and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.2 g, 0.5 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 12.1 g of Compound subB-16. (Yield: 65%, MS: [M+H]⁺=404)

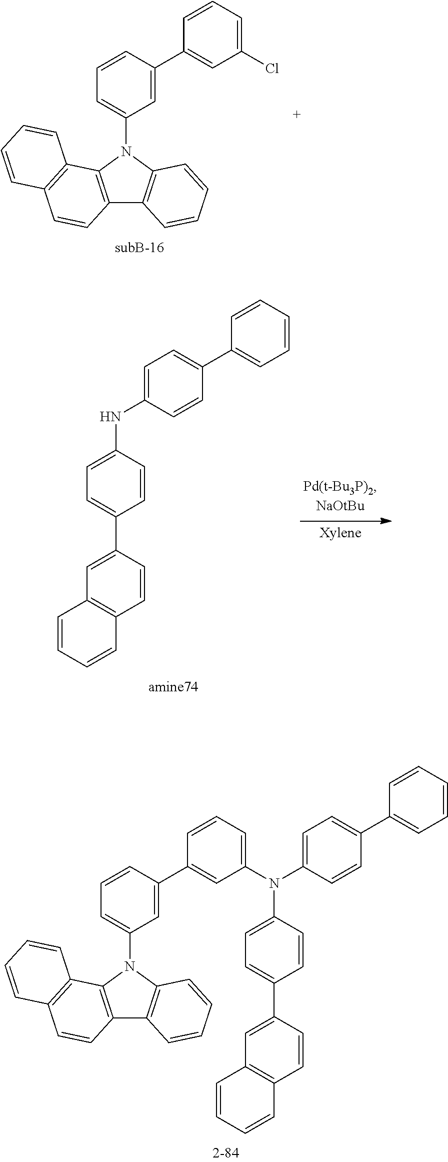 [SMILES: Clc1cccc(-c2cccc(-n3c4ccccc4c4ccc5ccccc5c43)c2)c1 ; c1ccc(-c2ccc(Nc3ccc(-c4ccc5ccccc5c4)cc3)cc2)cc1 ; c1ccc(-c2ccc(N(c3ccc(-c4ccc5ccccc5c4)cc3)c3cccc(-c4cccc(-n5c6ccccc6c6ccc7ccccc7c65)c4)c3)cc2)cc1]

Compound subB-16 (10 g, 24.8 mmol), Compound amine74 (9.7 g, 26 mmol) and sodium tert-butoxide (3.1 g, 32.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 12.2 g of Compound 2-84. (Yield: 67%, MS: [M+H]⁺=739)

### Synthesis Example 2-85

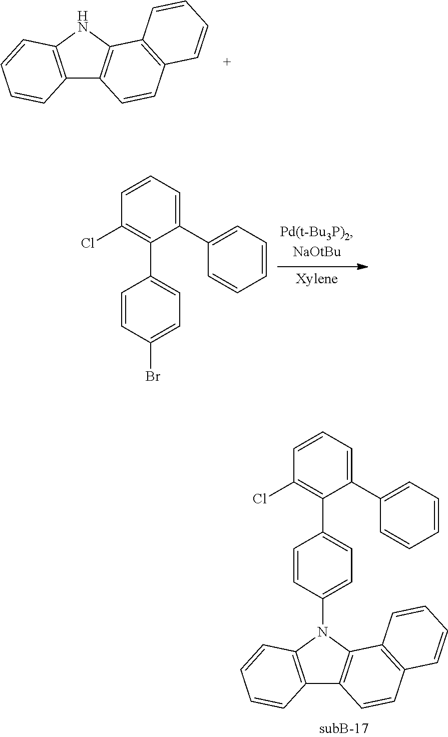 [SMILES: c1ccc2c(c1)ccc1c3ccccc3[nH]c21 ; Clc1cccc(-c2ccccc2)c1-c1ccc(Br)cc1 ; Clc1cccc(-c2ccccc2)c1-c1ccc(-n2c3ccccc3c3ccc4ccccc4c32)cc1]

11H-benzo[a]carbazole (10 g, 46 mmol), 4″-bromo-3′-chloro-1,1′:2′,1″-terphenyl (16.6 g, 48.3 mmol) and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.2 g, 0.5 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 14.6 g of Compound subB-17. (Yield: 66%, MS: [M+H]⁺=480)

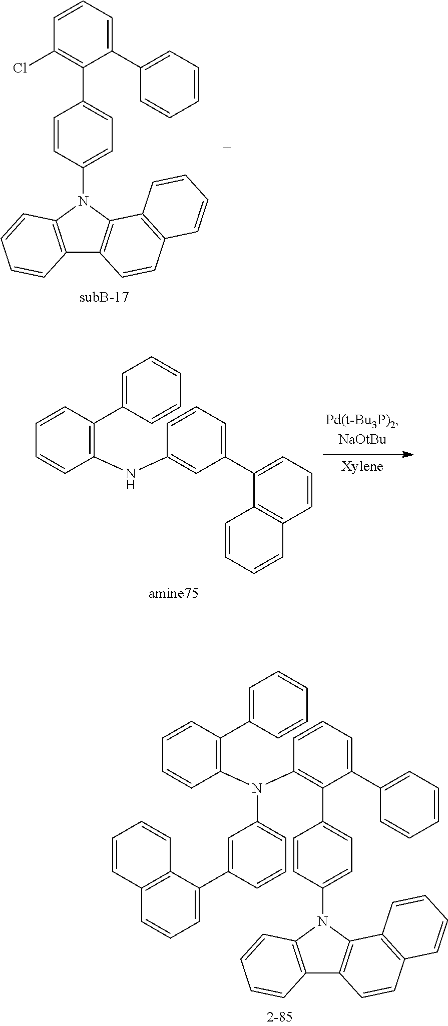 [SMILES: Clc1cccc(-c2ccccc2)c1-c1ccc(-n2c3ccccc3c3ccc4ccccc4c32)cc1 ; c1ccc(-c2ccccc2Nc2cccc(-c3cccc4ccccc34)c2)cc1 ; c1ccc(-c2ccccc2N(c2cccc(-c3cccc4ccccc34)c2)c2cccc(-c3ccccc3)c2-c2ccc(-n3c4ccccc4c4ccc5ccccc5c43)cc2)cc1]

Compound subB-17 (10 g, 20.8 mmol), Compound amine75 (7 g, 21.9 mmol) and sodium tert-butoxide (2.6 g, 27.1 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 11.2 g of Compound 2-85. (Yield: 66%, MS: [M+H]⁺=815)

### Synthesis Example 2-86

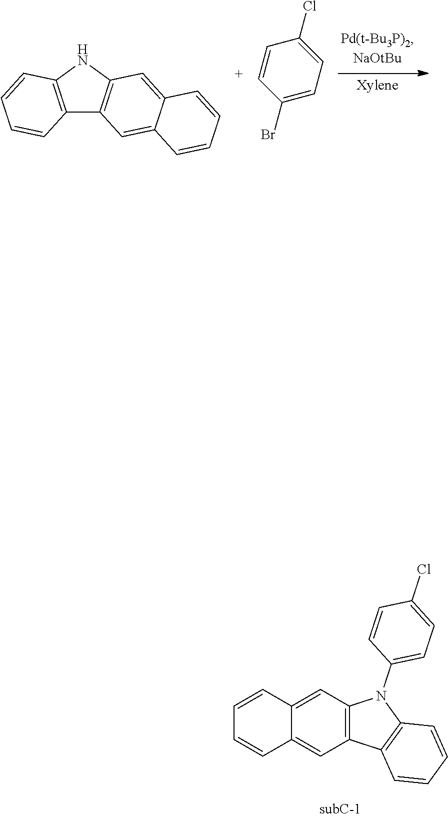 [SMILES: c1ccc2cc3c(cc2c1)[nH]c1ccccc13 ; Clc1ccc(Br)cc1 ; Clc1ccc(-n2c3ccccc3c3cc4ccccc4cc32)cc1]

5H-benzo[b]carbazole (10 g, 46 mmol), 1-bromo-4-chlorobenzene (9.3 g, 48.3 mmol) and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.2 g, 0.5 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 11.1 g of Compound subC-1. (Yield: 74%, MS: [M+H]⁺=328)

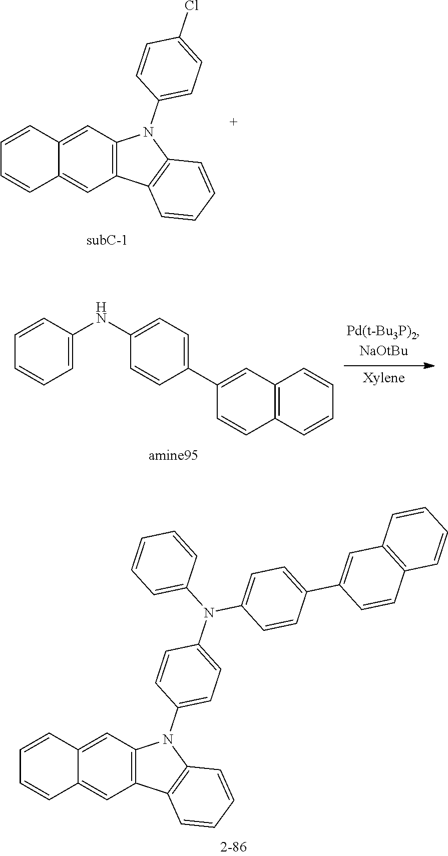 [SMILES: Clc1ccc(-n2c3ccccc3c3cc4ccccc4cc32)cc1 ; c1ccc(Nc2ccc(-c3ccc4ccccc4c3)cc2)cc1 ; c1ccc(N(c2ccc(-c3ccc4ccccc4c3)cc2)c2ccc(-n3c4ccccc4c4cc5ccccc5cc43)cc2)cc1]

Compound subC-1 (10 g, 30.5 mmol), Compound amine95 (9.5 g, 32 mmol) and sodium tert-butoxide (3.8 g, 39.7 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 10.9 g of Compound 2-86. (Yield: 61%, MS: [M+H]⁺=587)

### Synthesis Example 2-87

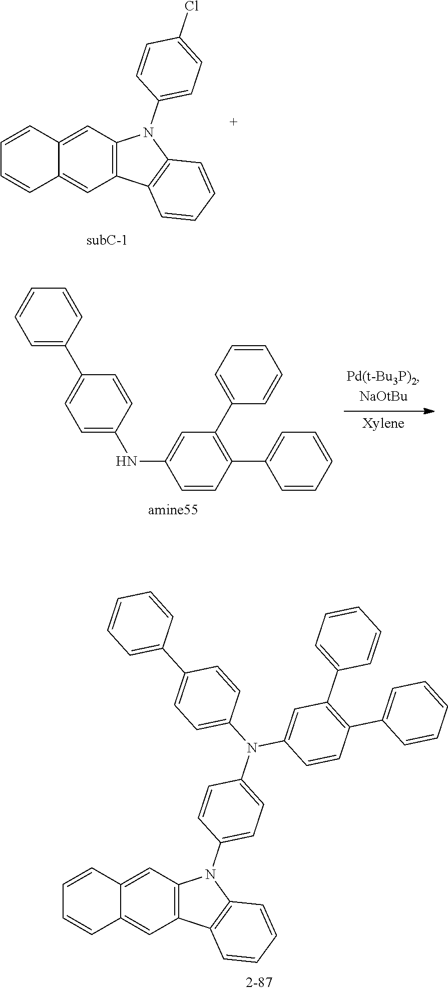 [SMILES: Clc1ccc(-n2c3ccccc3c3cc4ccccc4cc32)cc1 ; c1ccc(-c2ccc(Nc3ccc(-c4ccccc4)c(-c4ccccc4)c3)cc2)cc1 ; c1ccc(-c2ccc(N(c3ccc(-n4c5ccccc5c5cc6ccccc6cc54)cc3)c3ccc(-c4ccccc4)c(-c4ccccc4)c3)cc2)cc1]

Compound subC-1 (10 g, 30.5 mmol), Compound amine55 (12.7 g, 32 mmol) and sodium tert-butoxide (3.8 g, 39.7 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 14.9 g of Compound 2-87. (Yield: 71%, MS: [M+H]⁺=689)

### Synthesis Example 2-88

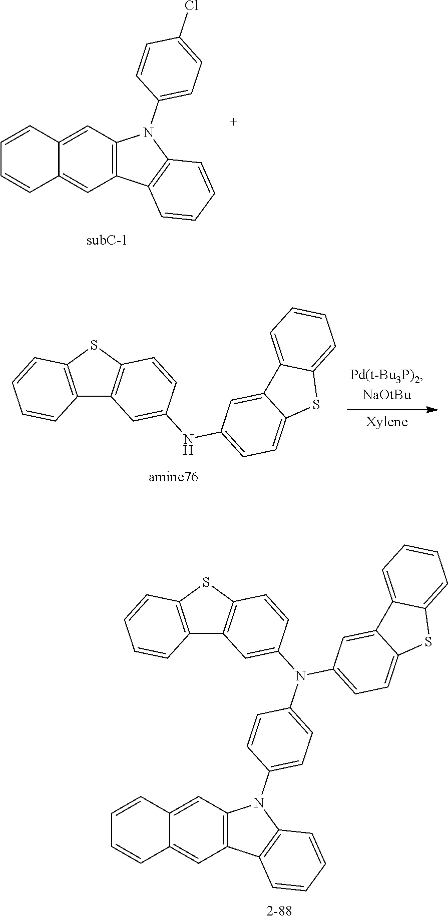 [SMILES: Clc1ccc(-n2c3ccccc3c3cc4ccccc4cc32)cc1 ; c1ccc2c(c1)sc1ccc(Nc3ccc4sc5ccccc5c4c3)cc12 ; c1ccc2cc3c(cc2c1)c1ccccc1n3-c1ccc(N(c2ccc3sc4ccccc4c3c2)c2ccc3sc4ccccc4c3c2)cc1]

Compound subC-1 (10 g, 30.5 mmol), Compound amine76 (12.2 g, 32 mmol) and sodium tert-butoxide (3.8 g, 39.7 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 21.6 g of Compound 2-88. (Yield: 73%, MS: [M+H]⁺=973)

### Synthesis Example 2-89

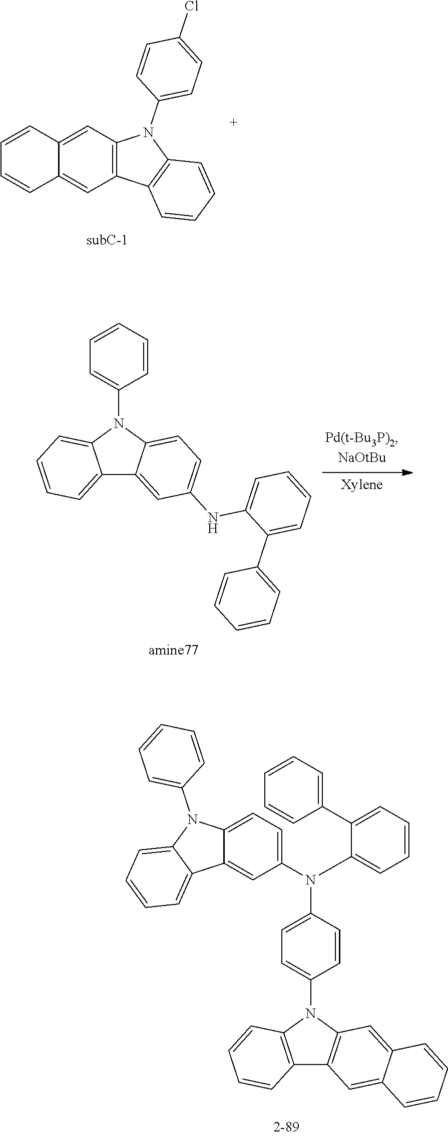 [SMILES: Clc1ccc(-n2c3ccccc3c3cc4ccccc4cc32)cc1 ; c1ccc(-c2ccccc2Nc2ccc3c(c2)c2ccccc2n3-c2ccccc2)cc1 ; c1ccc(-c2ccccc2N(c2ccc(-n3c4ccccc4c4cc5ccccc5cc43)cc2)c2ccc3c(c2)c2ccccc2n3-c2ccccc2)cc1]

Compound subC-1 (10 g, 30.5 mmol), Compound amine77 (13.1 g, 32 mmol) and sodium tert-butoxide (3.8 g, 39.7 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 14.1 g of Compound 2-89. (Yield: 66%, MS: [M+H]⁺=702)

### Synthesis Example 2-90

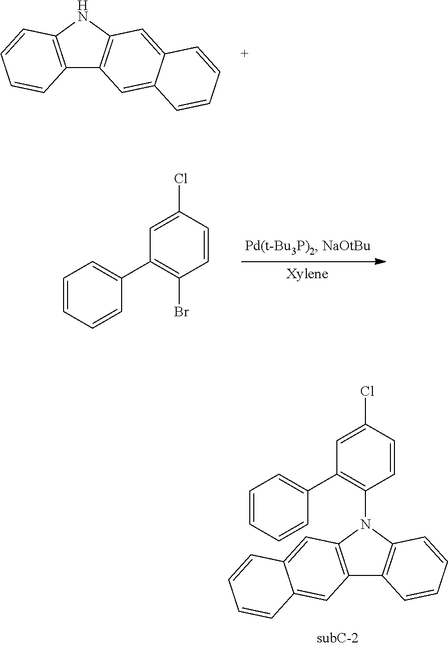 [SMILES: c1ccc2cc3c(cc2c1)[nH]c1ccccc13 ; Clc1ccc(Br)c(-c2ccccc2)c1 ; Clc1ccc(-n2c3ccccc3c3cc4ccccc4cc32)c(-c2ccccc2)c1]

5H-benzo[b]carbazole (10 g, 46 mmol), 2-bromo-5-chloro-1,1′-biphenyl (12.9 g, 48.3 mmol) and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.2 g, 0.5 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 11.3 g of Compound subC-2. (Yield: 61%, MS: [M+H]⁺=404)

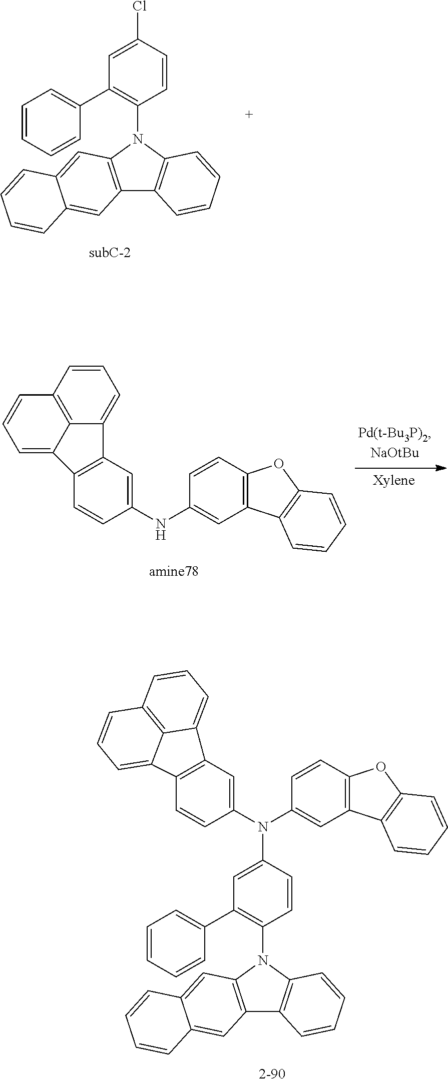 [SMILES: Clc1ccc(-n2c3ccccc3c3cc4ccccc4cc32)c(-c2ccccc2)c1 ; c1cc2c3c(cccc3c1)-c1cc(Nc3ccc4oc5ccccc5c4c3)ccc1-2 ; c1ccc(-c2cc(N(c3ccc4c(c3)-c3cccc5cccc-4c35)c3ccc4oc5ccccc5c4c3)ccc2-n2c3ccccc3c3cc4ccccc4cc32)cc1]

Compound subC-2 (10 g, 24.8 mmol), Compound amine78 (10 g, 26 mmol) and sodium tert-butoxide (3.1 g, 32.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 11.1 g of Compound 2-90. (Yield: 60%, MS: [M+H]⁺=751)

### Synthesis Example 2-91

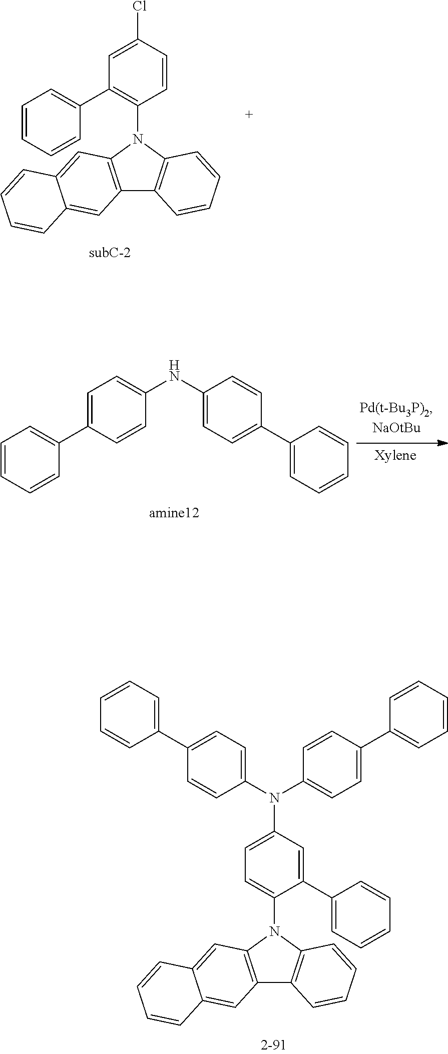 [SMILES: Clc1ccc(-n2c3ccccc3c3cc4ccccc4cc32)c(-c2ccccc2)c1 ; c1ccc(-c2ccc(Nc3ccc(-c4ccccc4)cc3)cc2)cc1 ; c1ccc(-c2ccc(N(c3ccc(-c4ccccc4)cc3)c3ccc(-n4c5ccccc5c5cc6ccccc6cc54)c(-c4ccccc4)c3)cc2)cc1]

Compound subC-2 (10 g, 24.8 mmol), Compound amine12 (8.4 g, 26 mmol amine78 (10 g, 26 mmol) and sodium tert-butoxide (3.1 g, 32.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 12.1 g of Compound 2-91. (Yield: 71%, MS: [M+H]⁺=689)

### Synthesis Example 2-92

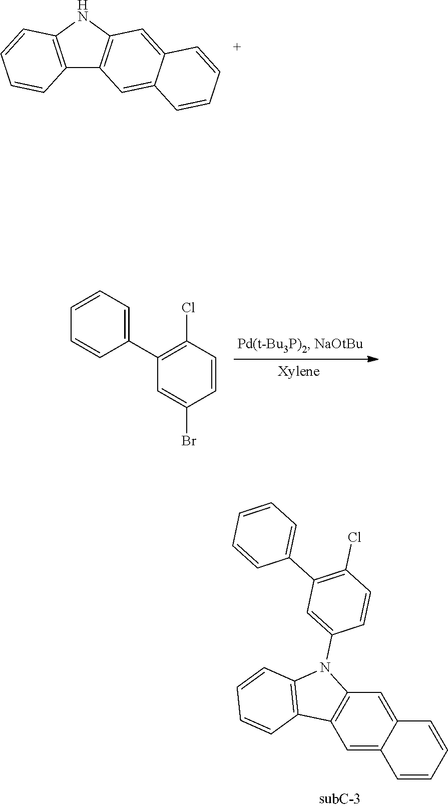 [SMILES: c1ccc2cc3c(cc2c1)[nH]c1ccccc13 ; Clc1ccc(Br)cc1-c1ccccc1 ; Clc1ccc(-n2c3ccccc3c3cc4ccccc4cc32)cc1-c1ccccc1]

5H-benzo[b]carbazole (10 g, 46 mmol), 2-chloro-5-bromo-1,1′-biphenyl (12.9 g, 48.3 mmol) and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.2 g, 0.5 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 11.3 g of Compound subC-3. (Yield: 61%, MS: [M+H]⁺=404)

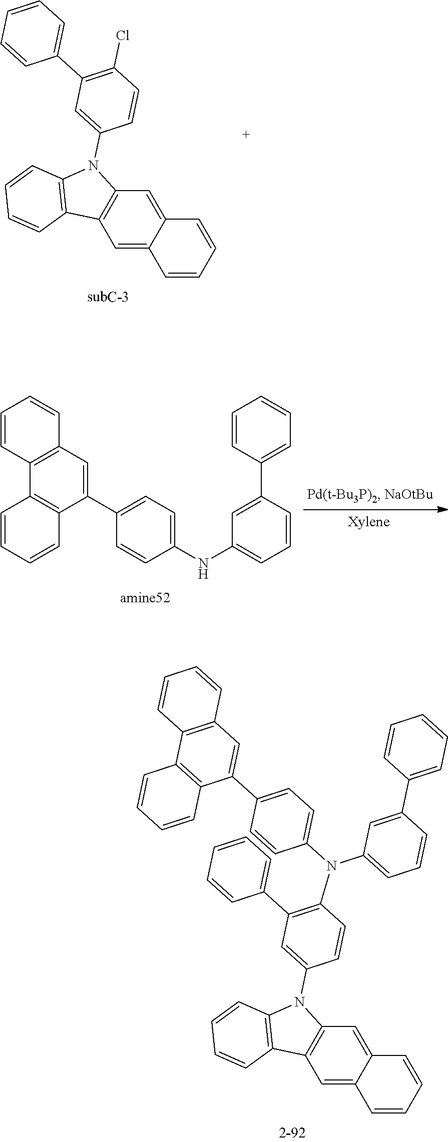 [SMILES: Clc1ccc(-n2c3ccccc3c3cc4ccccc4cc32)cc1-c1ccccc1 ; c1ccc(-c2cccc(Nc3ccc(-c4cc5ccccc5c5ccccc45)cc3)c2)cc1 ; c1ccc(-c2cccc(N(c3ccc(-c4cc5ccccc5c5ccccc45)cc3)c3ccc(-n4c5ccccc5c5cc6ccccc6cc54)cc3-c3ccccc3)c2)cc1]

Compound subC-3 (10 g, 24.8 mmol), Compound amine52 (6.4 g, 26 mmol) and sodium tert-butoxide (3.1 g, 32.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 10.3 g of Compound 2-92. (Yield: 68%, MS: [M+H]⁺=613)

### Synthesis Example 2-93

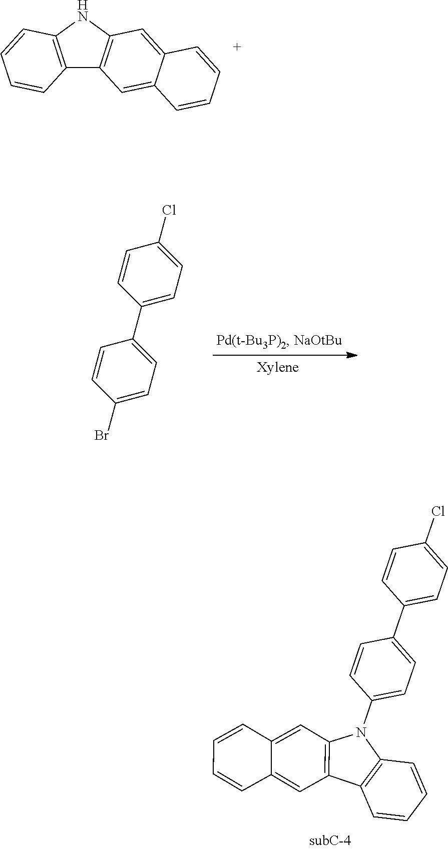 [SMILES: c1ccc2cc3c(cc2c1)[nH]c1ccccc13 ; Clc1ccc(-c2ccc(Br)cc2)cc1 ; Clc1ccc(-c2ccc(-n3c4ccccc4c4cc5ccccc5cc43)cc2)cc1]

5H-benzo[b]carbazole (10 g, 46 mmol), 4-bromo-4′-chloro-1,1′-biphenyl (12.9 g, 48.3 mmol) and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.2 g, 0.5 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 12.2 g of Compound subC-4. (Yield: 66%, MS: [M+H]⁺=404)

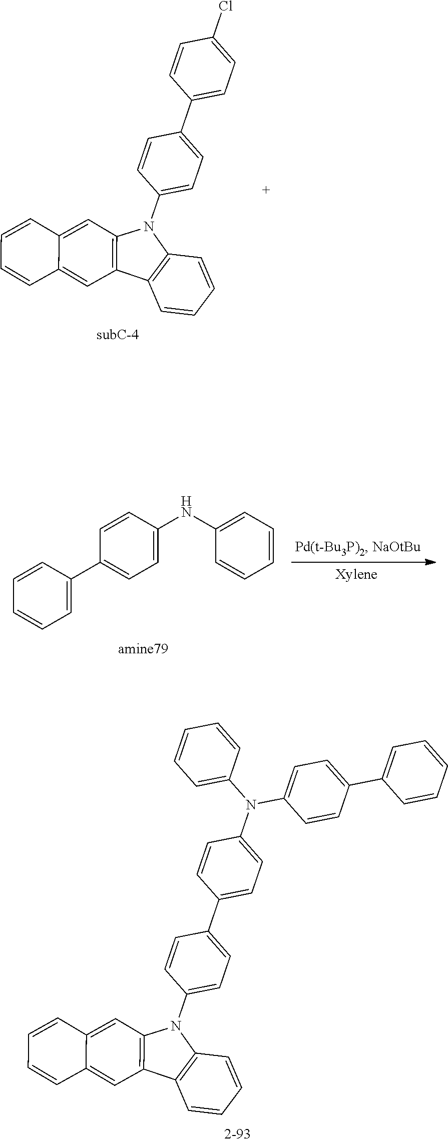 [SMILES: Clc1ccc(-c2ccc(-n3c4ccccc4c4cc5ccccc5cc43)cc2)cc1 ; c1ccc(Nc2ccc(-c3ccccc3)cc2)cc1 ; c1ccc(-c2ccc(N(c3ccccc3)c3ccc(-c4ccc(-n5c6ccccc6c6cc7ccccc7cc65)cc4)cc3)cc2)cc1]

Compound subC-4 (10 g, 24.8 mmol), Compound amine79 (6.4 g, 26 mmol) and sodium tert-butoxide (3.1 g, 32.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 9.5 g of Compound 2-93. (Yield: 63%, MS: [M+H]⁺=613)

### Synthesis Example 2-94

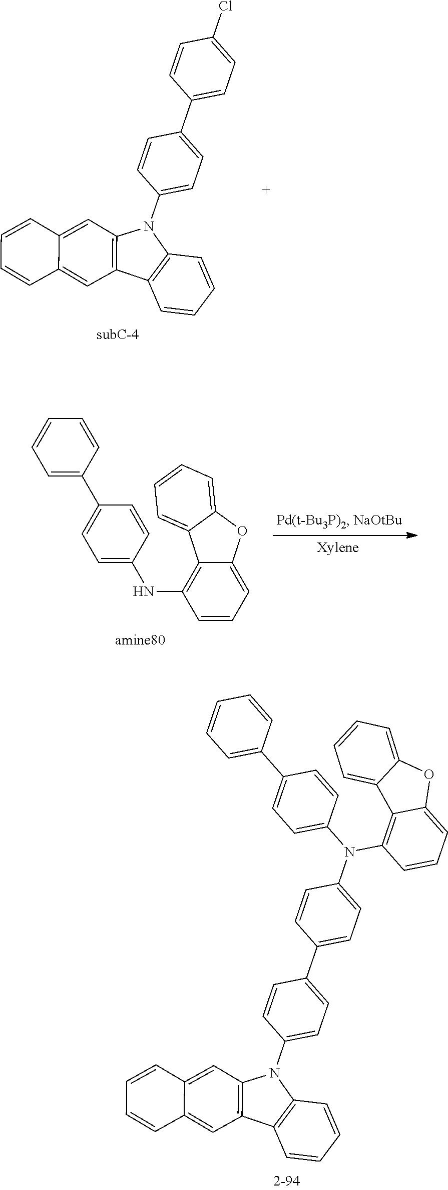 [SMILES: Clc1ccc(-c2ccc(-n3c4ccccc4c4cc5ccccc5cc43)cc2)cc1 ; c1ccc(-c2ccc(Nc3cccc4oc5ccccc5c34)cc2)cc1 ; c1ccc(-c2ccc(N(c3ccc(-c4ccc(-n5c6ccccc6c6cc7ccccc7cc65)cc4)cc3)c3cccc4oc5ccccc5c34)cc2)cc1]

Compound subC-4 (10 g, 24.8 mmol), Compound amine80 (8.7 g, 26 mmol) and sodium tert-butoxide (3.1 g, 32.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 11 g of Compound 2-94. (Yield: 63%, MS: [M+H]⁺=703)

### Synthesis Example 2-95

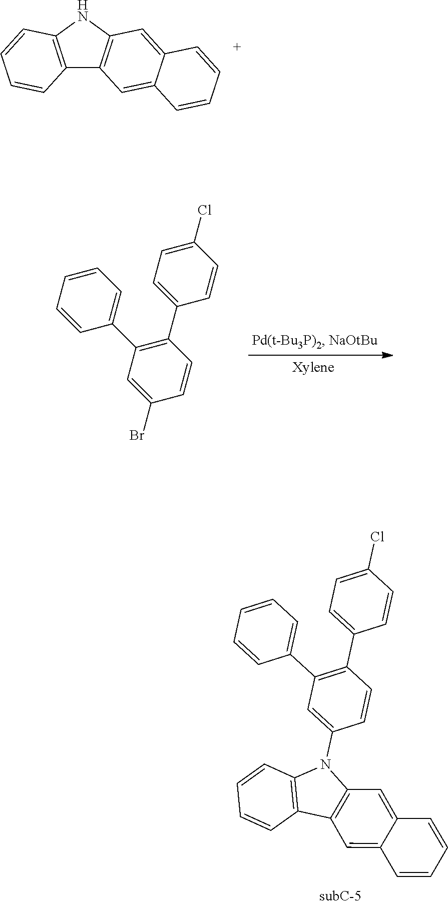 [SMILES: c1ccc2cc3c(cc2c1)[nH]c1ccccc13 ; Clc1ccc(-c2ccc(Br)cc2-c2ccccc2)cc1 ; Clc1ccc(-c2ccc(-n3c4ccccc4c4cc5ccccc5cc43)cc2-c2ccccc2)cc1]

5H-benzo[b]carbazole (10 g, 46 mmol), 4′-bromo-4-chloro-1,1′:2′,1″-terphenyl (16.6 g, 48.3 mmol) and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.2 g, 0.5 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.7 g of Compound subC-5. (Yield: 62%, MS: [M+H]⁺=480)

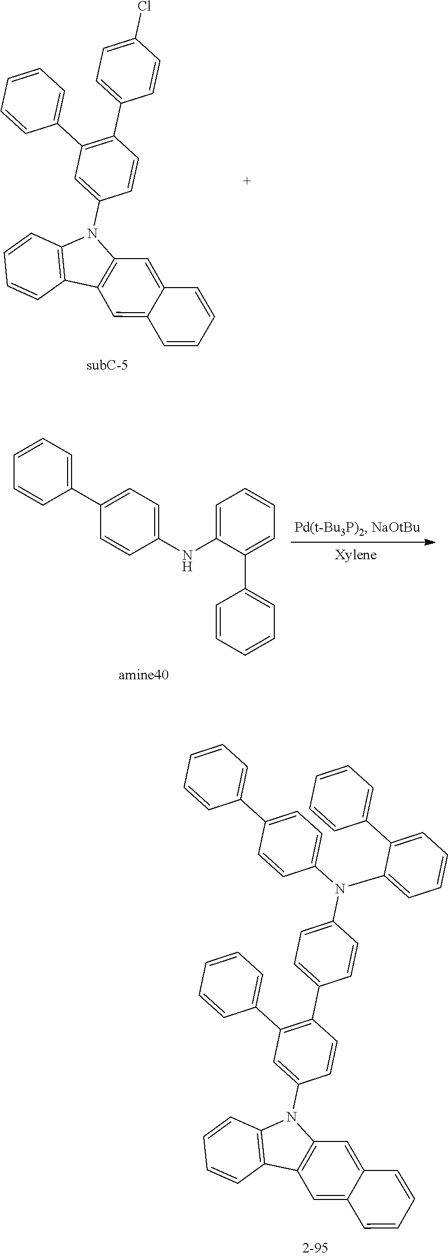 [SMILES: Clc1ccc(-c2ccc(-n3c4ccccc4c4cc5ccccc5cc43)cc2-c2ccccc2)cc1 ; c1ccc(-c2ccc(Nc3ccccc3-c3ccccc3)cc2)cc1 ; c1ccc(-c2ccc(N(c3ccc(-c4ccc(-n5c6ccccc6c6cc7ccccc7cc65)cc4-c4ccccc4)cc3)c3ccccc3-c3ccccc3)cc2)cc1]

Compound subC-5 (10 g, 20.8 mmol), Compound amine40 (7 g, 21.9 mmol) and sodium tert-butoxide (2.6 g, 27.1 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 10 g of Compound 2-95. (Yield: 64%, MS: [M+H]⁺=751)

### Synthesis Example 2-96

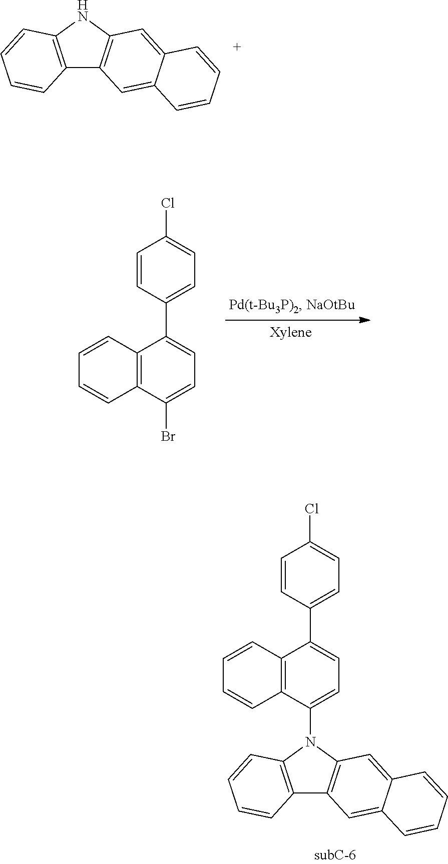 [SMILES: c1ccc2cc3c(cc2c1)[nH]c1ccccc13 ; Clc1ccc(-c2ccc(Br)c3ccccc23)cc1 ; Clc1ccc(-c2ccc(-n3c4ccccc4c4cc5ccccc5cc43)c3ccccc23)cc1]

5H-benzo[b]carbazole (10 g, 46 mmol), 1-bromo-4-(4-chlorophenyl) naphthalene (15.3 g, 48.3 mmol) and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.2 g, 0.5 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 15.4 g of Compound subC-6. (Yield: 74%, MS: [M+H]⁺=454)

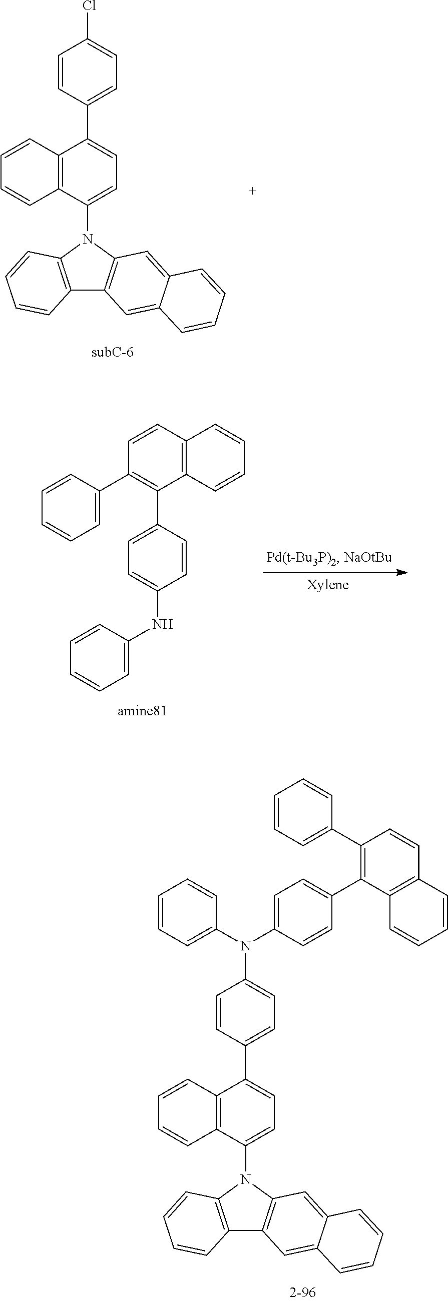 [SMILES: Clc1ccc(-c2ccc(-n3c4ccccc4c4cc5ccccc5cc43)c3ccccc23)cc1 ; c1ccc(Nc2ccc(-c3c(-c4ccccc4)ccc4ccccc34)cc2)cc1 ; c1ccc(-c2ccc3ccccc3c2-c2ccc(N(c3ccccc3)c3ccc(-c4ccc(-n5c6ccccc6c6cc7ccccc7cc65)c5ccccc45)cc3)cc2)cc1]

Compound subC-6 (10 g, 22 mmol), Compound amine81 (8.6 g, 23.1 mmol) and sodium tert-butoxide (2.8 g, 28.6 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 11.8 g of Compound 2-96. (Yield: 68%, MS: [M+H]⁺=789)

### Synthesis Example 2-97

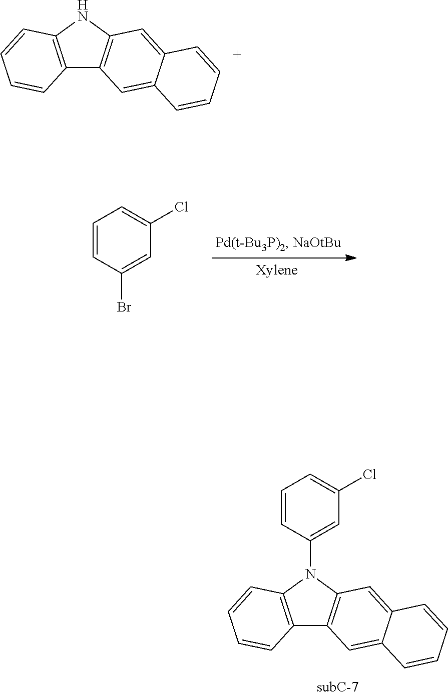 [SMILES: c1ccc2cc3c(cc2c1)[nH]c1ccccc13 ; Clc1cccc(Br)c1 ; Clc1cccc(-n2c3ccccc3c3cc4ccccc4cc32)c1]

5H-benzo[b]carbazole (10 g, 46 mmol), 1-bromo-3-chlorobenzene (9.3 g, 48.3 mmol) and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.2 g, 0.5 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 9 g of Compound subC-7. (Yield: 60%, MS: [M+H]⁺=328)

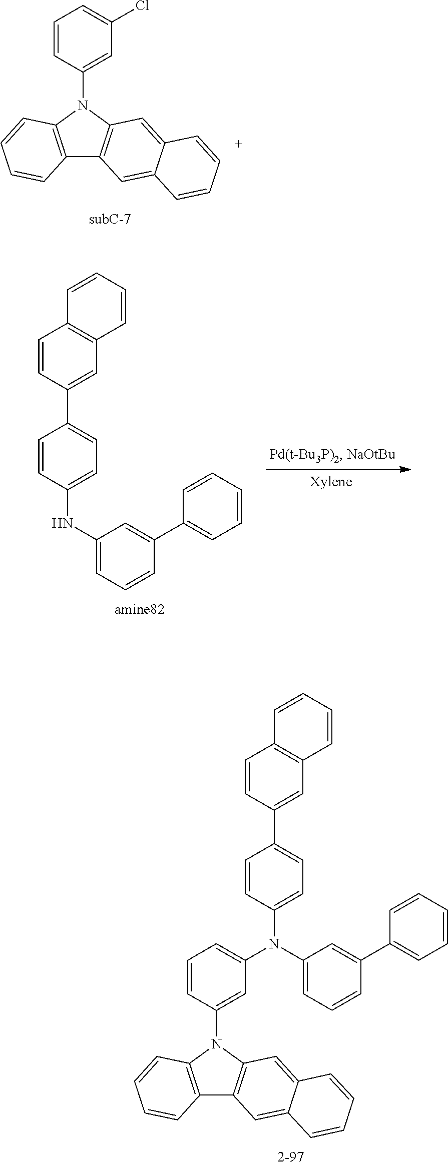 [SMILES: Clc1cccc(-n2c3ccccc3c3cc4ccccc4cc32)c1 ; c1ccc(-c2cccc(Nc3ccc(-c4ccc5ccccc5c4)cc3)c2)cc1 ; c1ccc(-c2cccc(N(c3ccc(-c4ccc5ccccc5c4)cc3)c3cccc(-n4c5ccccc5c5cc6ccccc6cc54)c3)c2)cc1]

Compound subC-7 (10 g, 30.5 mmol), Compound amine82 (11.9 g, 32 mmol) and sodium tert-butoxide (3.8 g, 39.7 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 14.3 g of Compound 2-97. (Yield: 71%, MS: [M+H]⁺=663)

### Synthesis Example 2-98

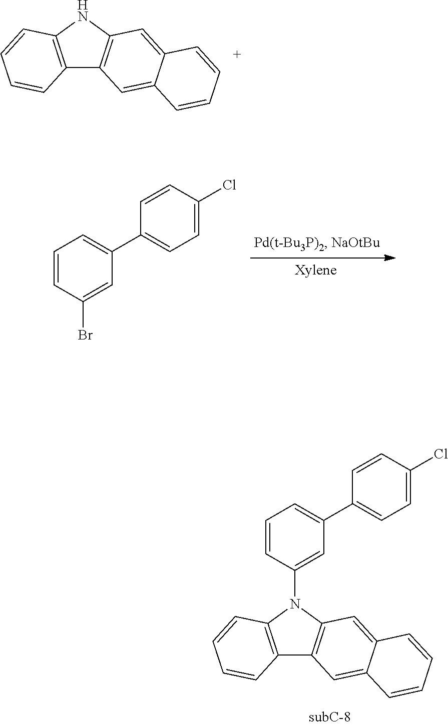 [SMILES: c1ccc2cc3c(cc2c1)[nH]c1ccccc13 ; Clc1ccc(-c2cccc(Br)c2)cc1 ; Clc1ccc(-c2cccc(-n3c4ccccc4c4cc5ccccc5cc43)c2)cc1]

5H-benzo[b]carbazole (10 g, 46 mmol), 3-bromo-4′-chloro-1,1′-biphenyl (12.9 g, 48.3 mmol) and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.2 g, 0.5 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare

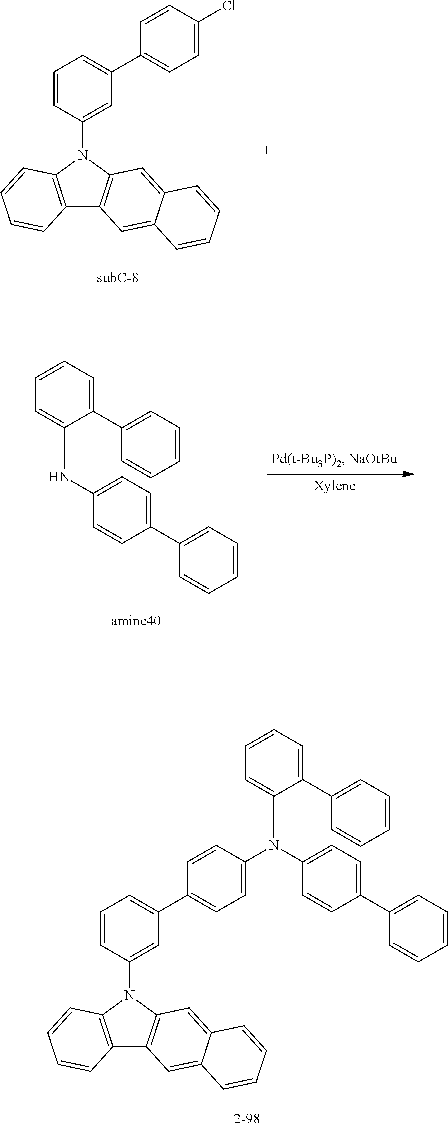 [SMILES: Clc1ccc(-c2cccc(-n3c4ccccc4c4cc5ccccc5cc43)c2)cc1 ; c1ccc(-c2ccc(Nc3ccccc3-c3ccccc3)cc2)cc1 ; c1ccc(-c2ccc(N(c3ccc(-c4cccc(-n5c6ccccc6c6cc7ccccc7cc65)c4)cc3)c3ccccc3-c3ccccc3)cc2)cc1]

11.7 g of Compound subC-8. (Yield: 63%, MS: [M+H]⁺=405) Compound subC-8 (10 g, 24.8 mmol), Compound amine40 (8.4 g, 26 mmol) and sodium tert-butoxide (3.1 g, 32.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 11.2 g of Compound 2-98. (Yield: 66%, MS: [M+H]⁺=689)

### Synthesis Example 2-99

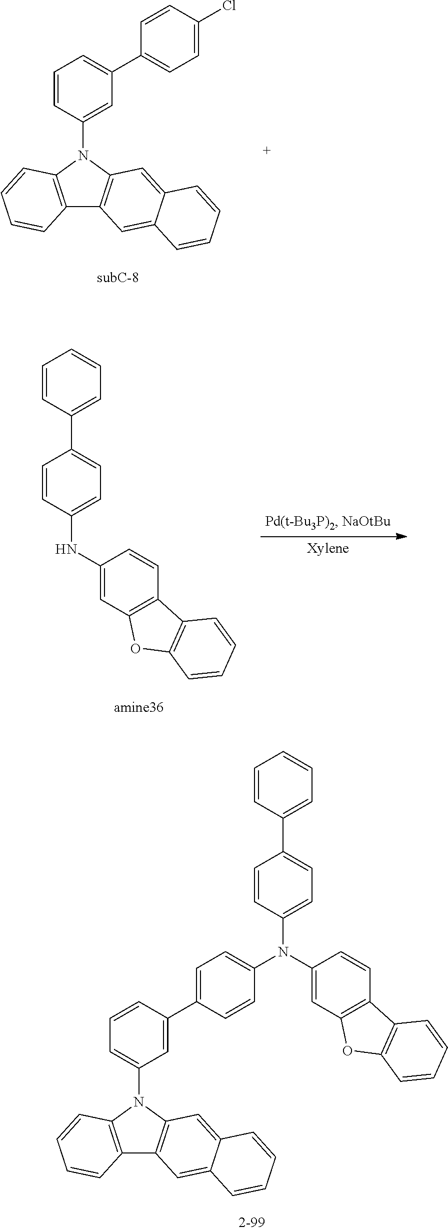 [SMILES: Clc1ccc(-c2cccc(-n3c4ccccc4c4cc5ccccc5cc43)c2)cc1 ; c1ccc(-c2ccc(Nc3ccc4c(c3)oc3ccccc34)cc2)cc1 ; c1ccc(-c2ccc(N(c3ccc(-c4cccc(-n5c6ccccc6c6cc7ccccc7cc65)c4)cc3)c3ccc4c(c3)oc3ccccc34)cc2)cc1]

Compound subC-8 (10 g, 24.8 mmol), Compound amine36 (8.7 g, 26 mmol) and sodium tert-butoxide (3.1 g, 32.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 10.4 g of Compound 2-99. (Yield: 60%, MS: [M+H]⁺=703)

### Synthesis Example 2-100

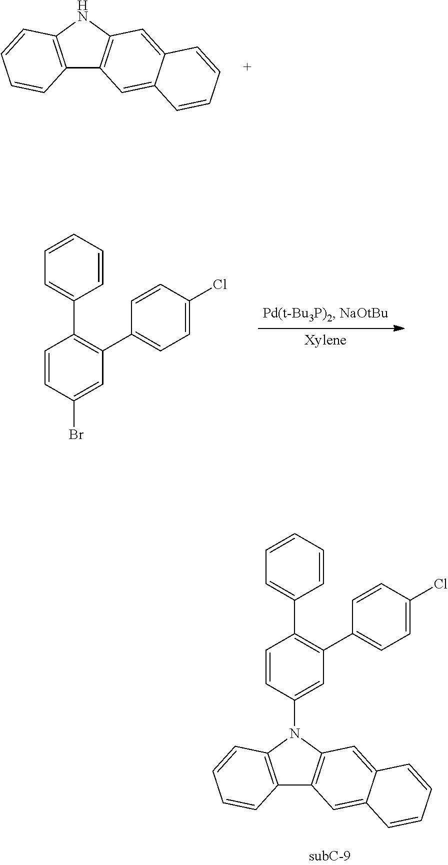 [SMILES: c1ccc2cc3c(cc2c1)[nH]c1ccccc13 ; Clc1ccc(-c2cc(Br)ccc2-c2ccccc2)cc1 ; Clc1ccc(-c2cc(-n3c4ccccc4c4cc5ccccc5cc43)ccc2-c2ccccc2)cc1]

5H-benzo[b]carbazole (10 g, 46 mmol), 5′-bromo-4-chloro-1,1′:2′,1″-terphenyl (16.6 g, 48.3 mmol) and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.2 g, 0.5 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 14.6 g of Compound subC-9. (Yield: 66%, MS: [M+H]⁺=480)

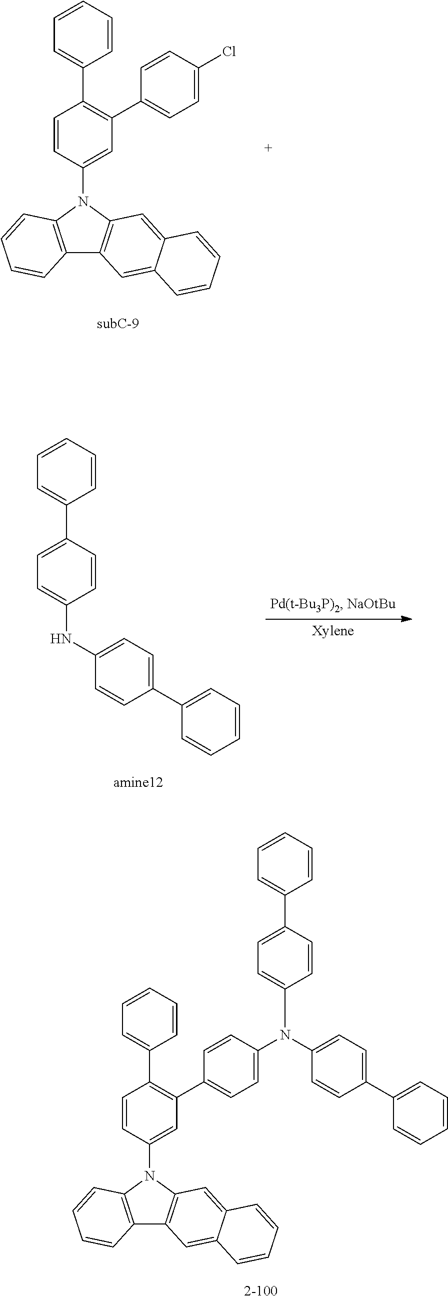 [SMILES: Clc1ccc(-c2cc(-n3c4ccccc4c4cc5ccccc5cc43)ccc2-c2ccccc2)cc1 ; c1ccc(-c2ccc(Nc3ccc(-c4ccccc4)cc3)cc2)cc1 ; c1ccc(-c2ccc(N(c3ccc(-c4ccccc4)cc3)c3ccc(-c4cc(-n5c6ccccc6c6cc7ccccc7cc65)ccc4-c4ccccc4)cc3)cc2)cc1]

Compound subC-9 (10 g, 20.8 mmol), Compound amine12 (7 g, 21.9 mmol) and sodium tert-butoxide (2.6 g, 27.1 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 11.3 g of Compound 2-100. (Yield: 71%, MS: [M+H]⁺=765)

### Synthesis Example 2-101

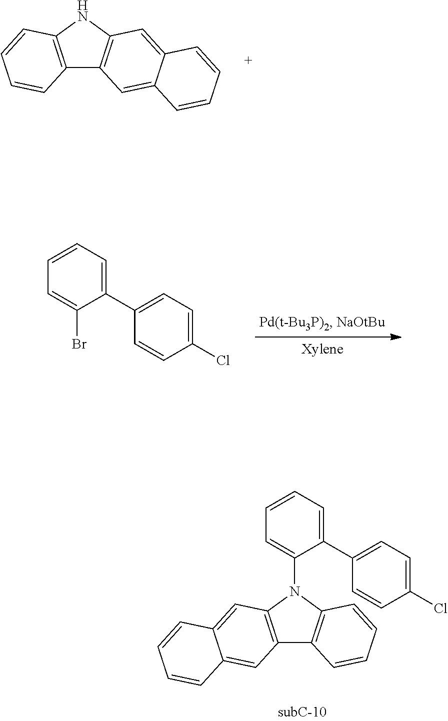 [SMILES: c1ccc2cc3c(cc2c1)[nH]c1ccccc13 ; Clc1ccc(-c2ccccc2Br)cc1 ; Clc1ccc(-c2ccccc2-n2c3ccccc3c3cc4ccccc4cc32)cc1]

5H-benzo[b]carbazole (10 g, 46 mmol), 2-bromo-4′-chloro-1,1′-biphenyl (12.9 g, 48.3 mmol) and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.2 g, 0.5 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 11.9 g of Compound subC-10. (Yield: 64%, MS: [M+H]⁺=404)

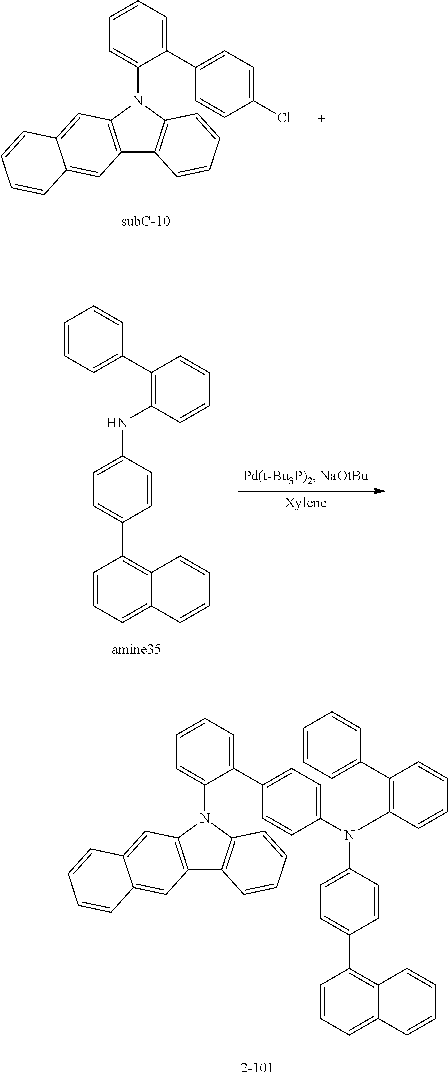 [SMILES: Clc1ccc(-c2ccccc2-n2c3ccccc3c3cc4ccccc4cc32)cc1 ; c1ccc(-c2ccccc2Nc2ccc(-c3cccc4ccccc34)cc2)cc1 ; c1ccc(-c2ccccc2N(c2ccc(-c3ccccc3-n3c4ccccc4c4cc5ccccc5cc43)cc2)c2ccc(-c3cccc4ccccc34)cc2)cc1]

Compound subC-10 (10 g, 24.8 mmol), Compound amine35 (9.7 g, 26 mmol) and sodium tert-butoxide (3.1 g, 32.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 12.1 g of Compound 2-101. (Yield: 66%, MS: [M+H]⁺=739)

### Synthesis Example 2-102

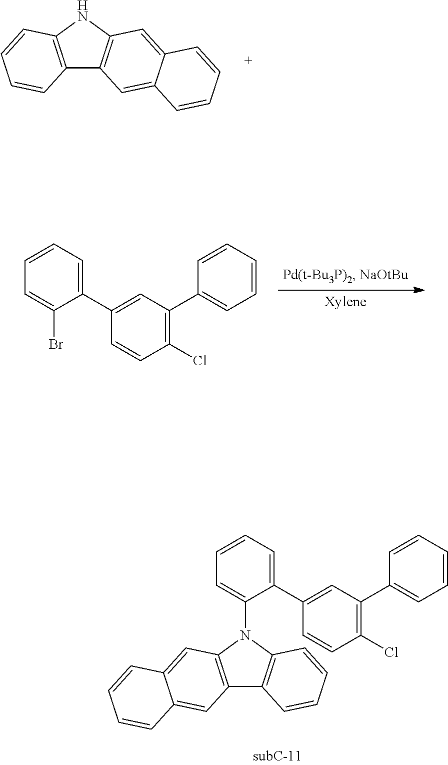 [SMILES: c1ccc2cc3c(cc2c1)[nH]c1ccccc13 ; Clc1ccc(-c2ccccc2Br)cc1-c1ccccc1 ; Clc1ccc(-c2ccccc2-n2c3ccccc3c3cc4ccccc4cc32)cc1-c1ccccc1]

5H-benzo[b]carbazole (10 g, 46 mmol), 2-bromo-4′-chloro-1,1′:3′,1″-terphenyl (16.6 g, 48.3 mmol) and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.2 g, 0.5 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.9 g f Compound subC-11. (Yield: 63%, MS: [M+H]⁺=480)

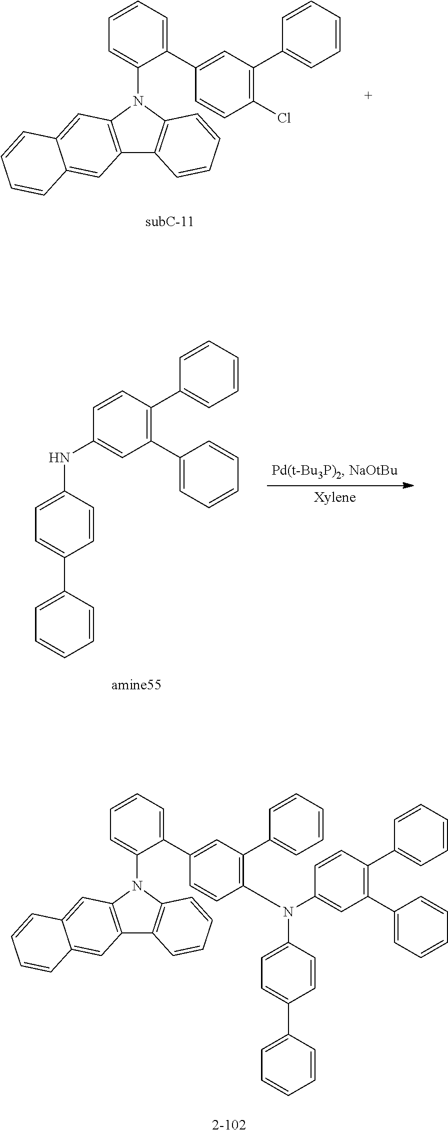 [SMILES: Clc1ccc(-c2ccccc2-n2c3ccccc3c3cc4ccccc4cc32)cc1-c1ccccc1 ; c1ccc(-c2ccc(Nc3ccc(-c4ccccc4)c(-c4ccccc4)c3)cc2)cc1 ; c1ccc(-c2ccc(N(c3ccc(-c4ccccc4)c(-c4ccccc4)c3)c3ccc(-c4ccccc4-n4c5ccccc5c5cc6ccccc6cc54)cc3-c3ccccc3)cc2)cc1]

Compound subC-11 (10 g, 20.8 mmol), Compound amine55 (8.6 g, 21.9 mmol) and sodium tert-butoxide (2.6 g, 27.1 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 11.2 g of Compound 2-102. (Yield: 64%, MS: [M+H]⁺=841)

### Synthesis Example 2-103

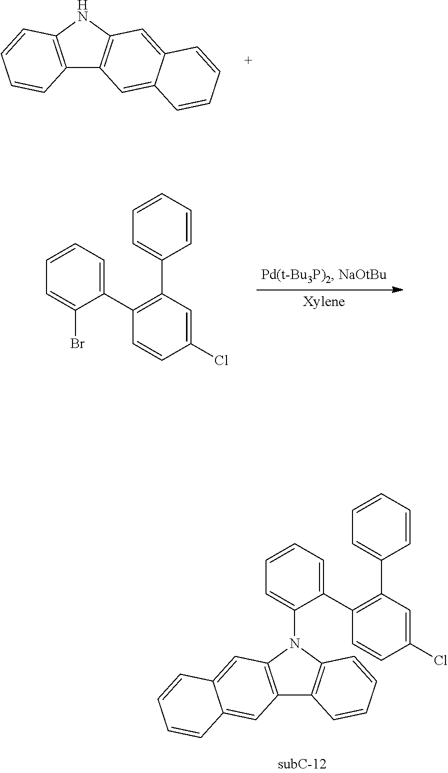 [SMILES: c1ccc2cc3c(cc2c1)[nH]c1ccccc13 ; Clc1ccc(-c2ccccc2Br)c(-c2ccccc2)c1 ; Clc1ccc(-c2ccccc2-n2c3ccccc3c3cc4ccccc4cc32)c(-c2ccccc2)c1]

5H-benzo[b]carbazole (10 g, 46 mmol), 2-bromo-4′-chloro-1,1′:2′,1″-terphenyl (16.6 g, 48.3 mmol) and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.2 g, 0.5 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 15.2 g of Compound subC-12. (Yield: 69%, MS: [M+H]⁺=480)

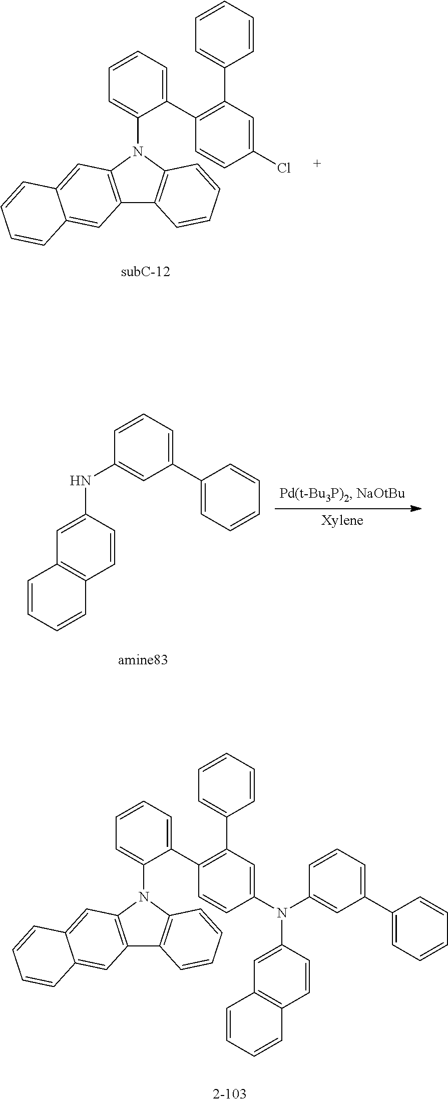 [SMILES: Clc1ccc(-c2ccccc2-n2c3ccccc3c3cc4ccccc4cc32)c(-c2ccccc2)c1 ; c1ccc(-c2cccc(Nc3ccc4ccccc4c3)c2)cc1 ; c1ccc(-c2cccc(N(c3ccc(-c4ccccc4-n4c5ccccc5c5cc6ccccc6cc54)c(-c4ccccc4)c3)c3ccc4ccccc4c3)c2)cc1]

Compound subC-12 (10 g, 20.8 mmol), Compound amine83 (6.5 g, 21.9 mmol) and sodium tert-butoxide (2.6 g, 27.1 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 11.4 g of Compound 2-103. (Yield: 74%, MS: [M+H]⁺=739)

### Synthesis Example 2-104

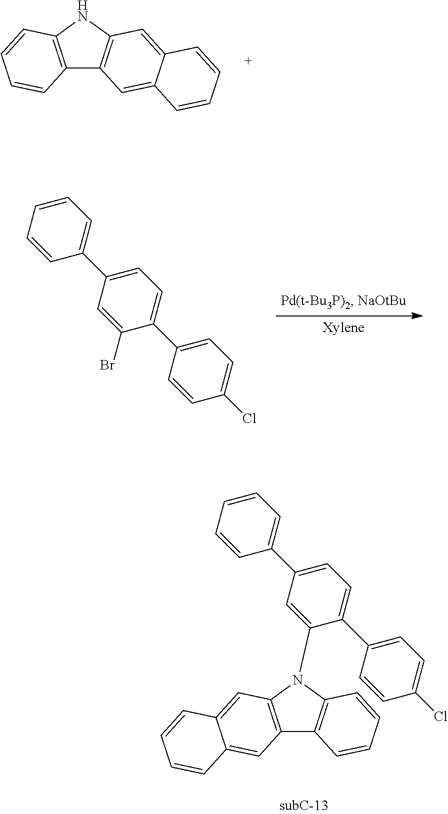 [SMILES: c1ccc2cc3c(cc2c1)[nH]c1ccccc13 ; Clc1ccc(-c2ccc(-c3ccccc3)cc2Br)cc1 ; Clc1ccc(-c2ccc(-c3ccccc3)cc2-n2c3ccccc3c3cc4ccccc4cc32)cc1]

5H-benzo[b]carbazole (10 g, 46 mmol), 2′-bromo-4-chloro-1,1′:4′,1″-terphenyl (16.6 g, 48.3 mmol) and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.2 g, 0.5 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 14.6 g of Compound subC-13. (Yield: 66%, MS: [M+H]⁺=480)

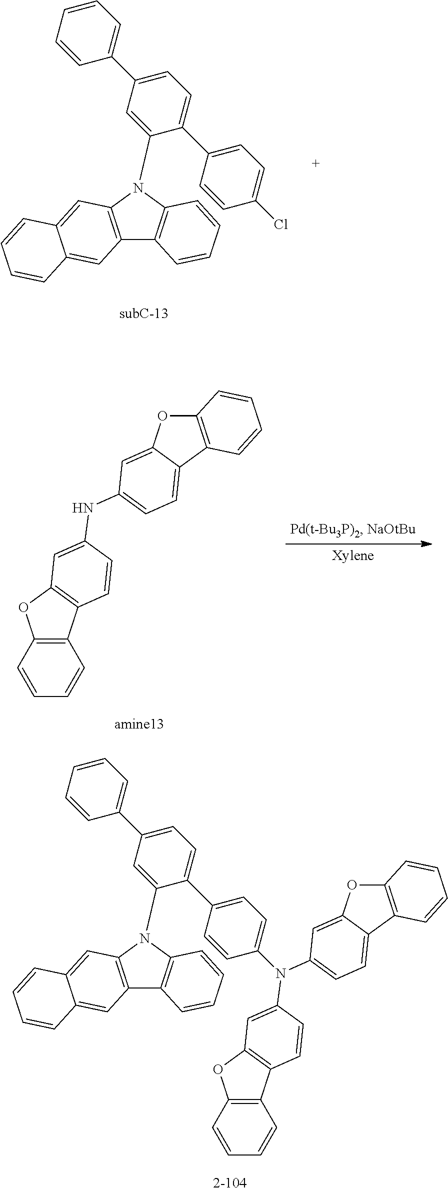 [SMILES: Clc1ccc(-c2ccc(-c3ccccc3)cc2-n2c3ccccc3c3cc4ccccc4cc32)cc1 ; c1ccc2c(c1)oc1cc(Nc3ccc4c(c3)oc3ccccc34)ccc12 ; c1ccc(-c2ccc(-c3ccc(N(c4ccc5c(c4)oc4ccccc45)c4ccc5c(c4)oc4ccccc45)cc3)c(-n3c4ccccc4c4cc5ccccc5cc43)c2)cc1]

Compound subC-13 (10 g, 20.8 mmol), Compound amine13 (7.6 g, 21.9 mmol) and sodium tert-butoxide (2.6 g, 27.1 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 10.9 g of Compound 2-104. (Yield: 66%, MS: [M+H]⁺=793)

### Synthesis Example 2-105

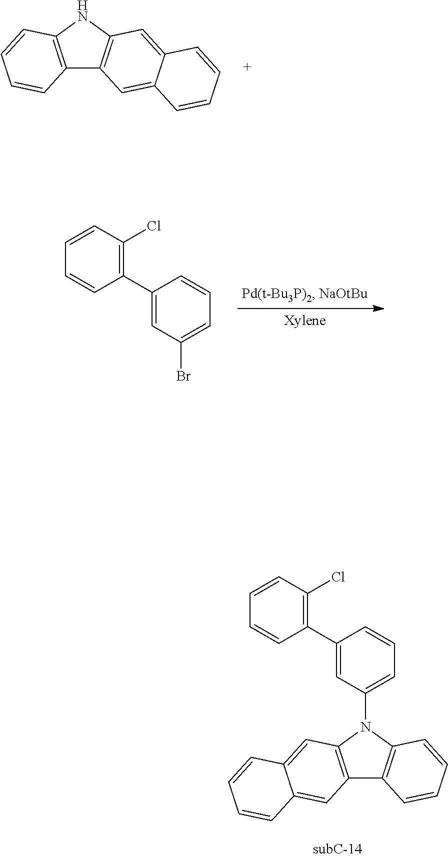 [SMILES: c1ccc2cc3c(cc2c1)[nH]c1ccccc13 ; Clc1ccccc1-c1cccc(Br)c1 ; Clc1ccccc1-c1cccc(-n2c3ccccc3c3cc4ccccc4cc32)c1]

5H-benzo[b]carbazole (10 g, 46 mmol), 3′-bromo-2-chloro-1,1′-biphenyl (12.9 g, 48.3 mmol) and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.2 g, 0.5 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.9 g of Compound subC-14. (Yield: 75%, MS: [M+H]⁺=405)

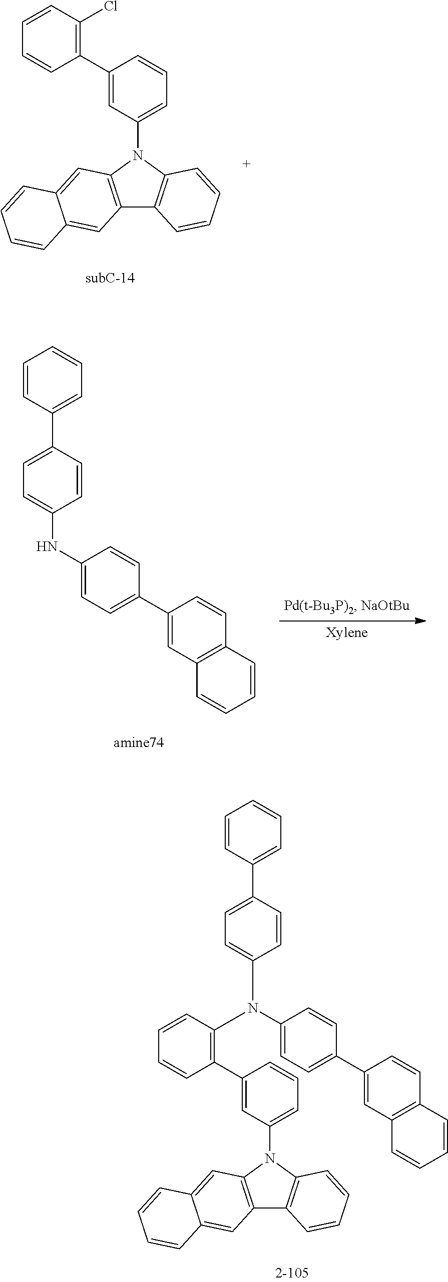 [SMILES: Clc1ccccc1-c1cccc(-n2c3ccccc3c3cc4ccccc4cc32)c1 ; c1ccc(-c2ccc(Nc3ccc(-c4ccc5ccccc5c4)cc3)cc2)cc1 ; c1ccc(-c2ccc(N(c3ccc(-c4ccc5ccccc5c4)cc3)c3ccccc3-c3cccc(-n4c5ccccc5c5cc6ccccc6cc54)c3)cc2)cc1]

Compound subC-14 (10 g, 24.8 mmol), Compound amine74 (9.7 g, 26 mmol) and sodium tert-butoxide (3.1 g, 32.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.2 g of Compound 2-105. (Yield: 72%, MS: [M+H]⁺=739)

### Synthesis Example 2-106

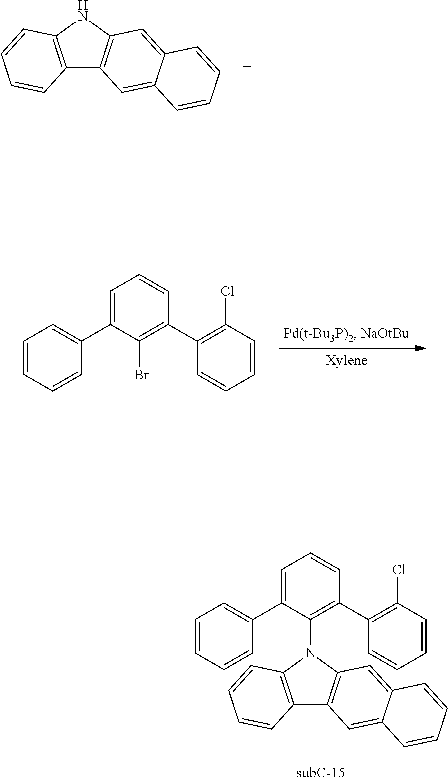 [SMILES: c1ccc2cc3c(cc2c1)[nH]c1ccccc13 ; Clc1ccccc1-c1cccc(-c2ccccc2)c1Br ; Clc1ccccc1-c1cccc(-c2ccccc2)c1-n1c2ccccc2c2cc3ccccc3cc21]

5H-benzo[b]carbazole (10 g, 46 mmol), 2′-bromo-2-chloro-1,1′:3′,1″-terphenyl (16.6 g, 48.3 mmol) and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.2 g, 0.5 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 14.1 g of Compound subC-15. (Yield: 64%, MS: [M+H]⁺=480)

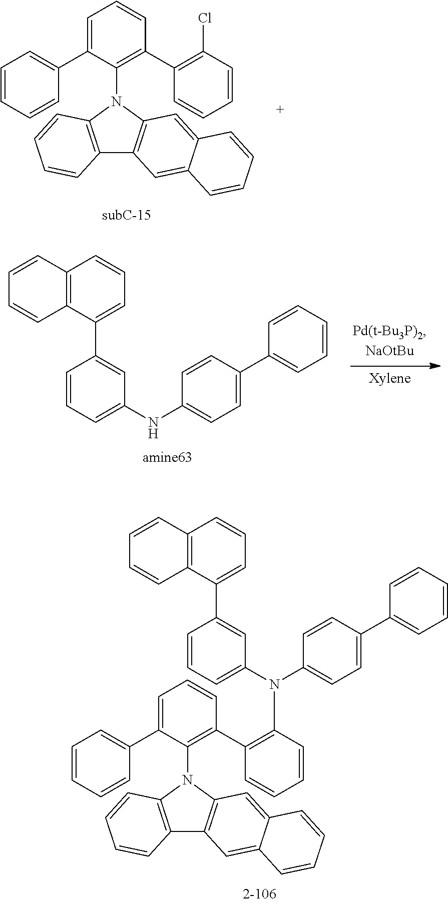 [SMILES: Clc1ccccc1-c1cccc(-c2ccccc2)c1-n1c2ccccc2c2cc3ccccc3cc21 ; c1ccc(-c2ccc(Nc3cccc(-c4cccc5ccccc45)c3)cc2)cc1 ; c1ccc(-c2ccc(N(c3cccc(-c4cccc5ccccc45)c3)c3ccccc3-c3cccc(-c4ccccc4)c3-n3c4ccccc4c4cc5ccccc5cc43)cc2)cc1]

Compound subC-15 (10 g, 20.8 mmol), Compound amine63 (8.1 g, 21.9 mmol) and sodium tert-butoxide (2.6 g, 27.1 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 11 g of Compound 2-106. (Yield: 65%, MS: [M+H]⁺=815)

### Synthesis Example 2-107

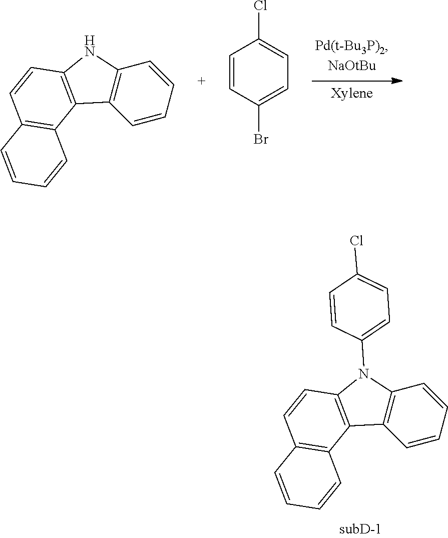 [SMILES: c1ccc2c(c1)ccc1[nH]c3ccccc3c12 ; Clc1ccc(Br)cc1 ; Clc1ccc(-n2c3ccccc3c3c4ccccc4ccc32)cc1]

7H-benzo[c]carbazole (10 g, 46 mmol), 1-bromo-4-chlorobenzene (9.3 g, 48.3 mmol) and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.2 g, 0.5 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 10.8 g of Compound subD-1. (Yield: 72%, MS: [M+H]⁺=328)

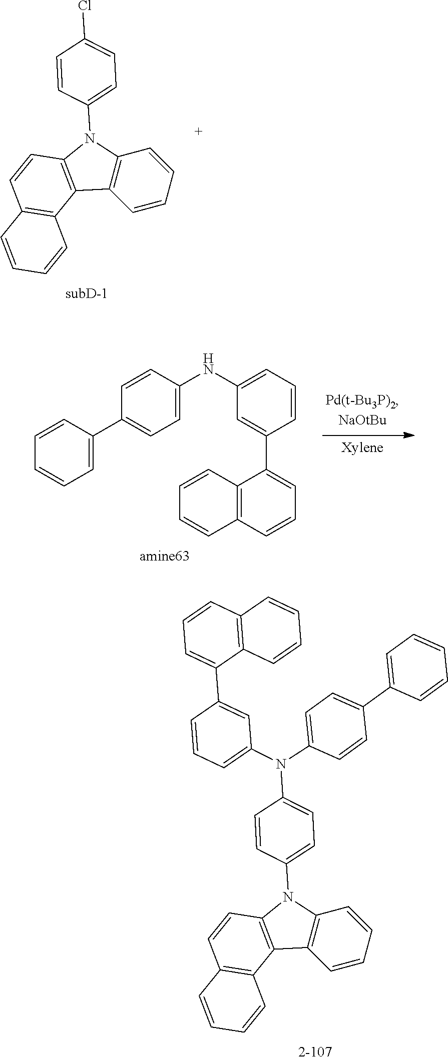 [SMILES: Clc1ccc(-n2c3ccccc3c3c4ccccc4ccc32)cc1 ; c1ccc(-c2ccc(Nc3cccc(-c4cccc5ccccc45)c3)cc2)cc1 ; c1ccc(-c2ccc(N(c3ccc(-n4c5ccccc5c5c6ccccc6ccc54)cc3)c3cccc(-c4cccc5ccccc45)c3)cc2)cc1]

Compound subD-1 (10 g, 30.5 mmol), Compound amine63 (11.9 g, 32 mmol) and sodium tert-butoxide (3.8 g, 39.7 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 14.5 g of Compound 2-107. (Yield: 72%, MS: [M+H]⁺=663)

### Synthesis Example 2-108

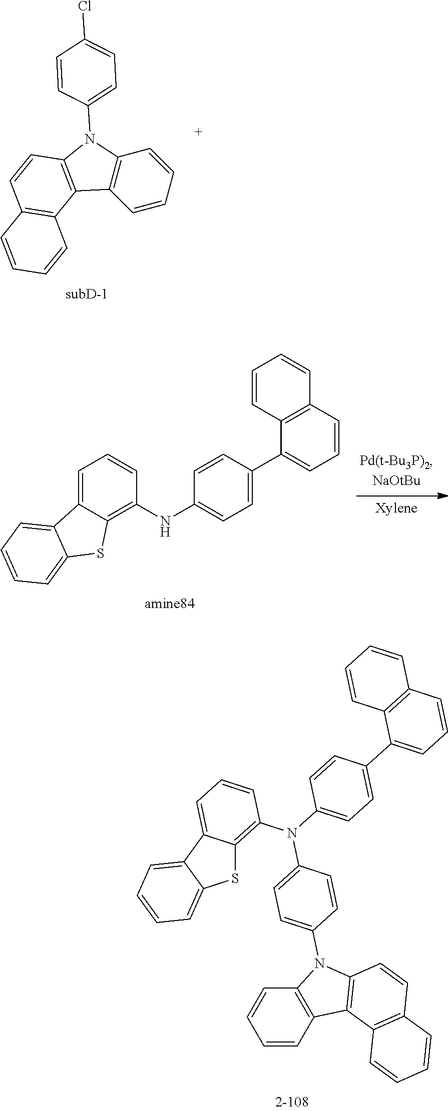 [SMILES: Clc1ccc(-n2c3ccccc3c3c4ccccc4ccc32)cc1 ; c1ccc2c(-c3ccc(Nc4cccc5c4sc4ccccc45)cc3)cccc2c1 ; c1ccc2c(-c3ccc(N(c4ccc(-n5c6ccccc6c6c7ccccc7ccc65)cc4)c4cccc5c4sc4ccccc45)cc3)cccc2c1]

Compound subD-1 (10 g, 30.5 mmol), Compound amine84 (12.9 g, 32 mmol) and sodium tert-butoxide (3.8 g, 39.7 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 15.4 g of Compound 2-108. (Yield: 73%, MS: [M+H]⁺=693)

### Synthesis Example 2-109

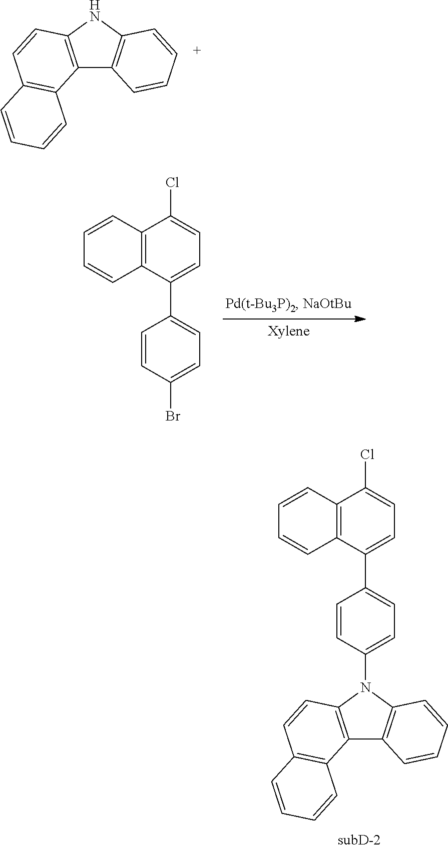 [SMILES: c1ccc2c(c1)ccc1[nH]c3ccccc3c12 ; Clc1ccc(-c2ccc(Br)cc2)c2ccccc12 ; Clc1ccc(-c2ccc(-n3c4ccccc4c4c5ccccc5ccc43)cc2)c2ccccc12]

7H-benzo[c]carbazole (10 g, 46 mmol), 1-(4-bromophenyl)-4-chloronaphthalene (15.3 g, 48.3 mmol) and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.2 g, 0.5 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 14.8 g of Compound subD-2. (Yield: 71%, MS: [M+H]⁺=454)

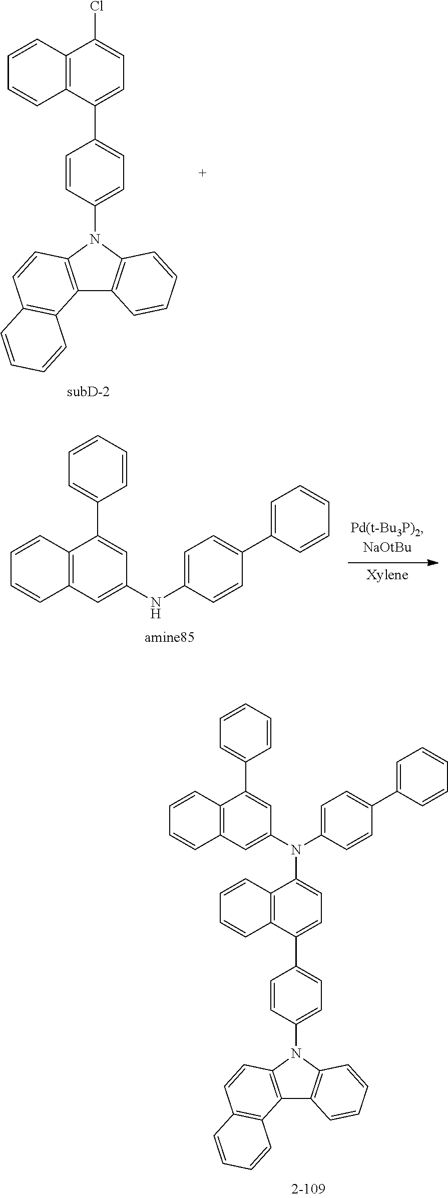 [SMILES: Clc1ccc(-c2ccc(-n3c4ccccc4c4c5ccccc5ccc43)cc2)c2ccccc12 ; c1ccc(-c2ccc(Nc3cc(-c4ccccc4)c4ccccc4c3)cc2)cc1 ; c1ccc(-c2ccc(N(c3cc(-c4ccccc4)c4ccccc4c3)c3ccc(-c4ccc(-n5c6ccccc6c6c7ccccc7ccc65)cc4)c4ccccc34)cc2)cc1]

Compound subD-2 (10 g, 22 mmol), Compound amine85 (8.6 g, 23.1 mmol) and sodium tert-butoxide (2.8 g, 28.6 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 10.8 g of Compound 2-109. (Yield: 62%, MS: [M+H]⁺=789)

### Synthesis Example 2-110

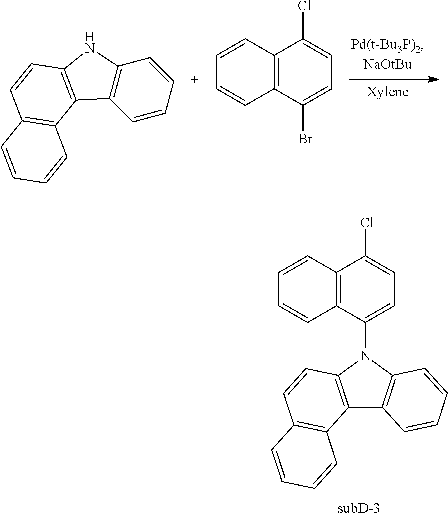

7H-benzo[c]carbazole (10 g, 46 mmol), 1-bromo-4-chloronaphthalene (11.7 g, 48.3 mmol) and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.2 g, 0.5 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 12.3 g of Compound subD-3. (Yield: 71%, MS: [M+H]⁺=378)

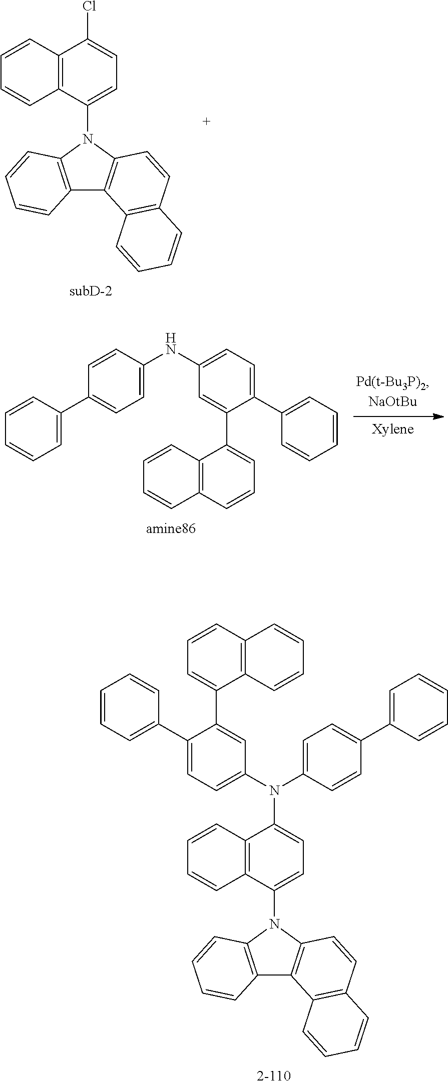 [SMILES: Clc1ccc(-n2c3ccccc3c3c4ccccc4ccc32)c2ccccc12 ; c1ccc(-c2ccc(Nc3ccc(-c4ccccc4)c(-c4cccc5ccccc45)c3)cc2)cc1 ; c1ccc(-c2ccc(N(c3ccc(-c4ccccc4)c(-c4cccc5ccccc45)c3)c3ccc(-n4c5ccccc5c5c6ccccc6ccc54)c4ccccc34)cc2)cc1]

Compound subD-3 (10 g, 26.5 mmol), Compound amine86 (12.4 g, 27.8 mmol) and sodium tert-butoxide (3.3 g, 34.4 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 14.6 g of Compound 2-110. (Yield: 70%, MS: [M+H]⁺=789)

### Synthesis Example 2-111

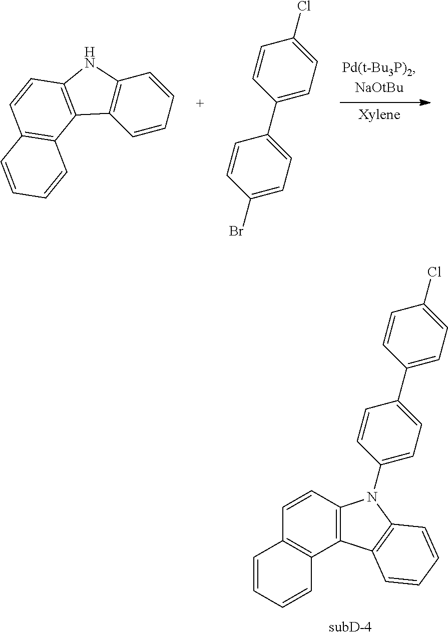 [SMILES: c1ccc2c(c1)ccc1[nH]c3ccccc3c12 ; Clc1ccc(-c2ccc(Br)cc2)cc1 ; Clc1ccc(-c2ccc(-n3c4ccccc4c4c5ccccc5ccc43)cc2)cc1]

7H-benzo[c]carbazole (10 g, 46 mmol), 4-bromo-4′-chloro-1,1′-biphenyl (12.9 g, 48.3 mmol) and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.2 g, 0.5 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.7 g of Compound subD-4. (Yield: 74%, MS: [M+H]⁺=404)

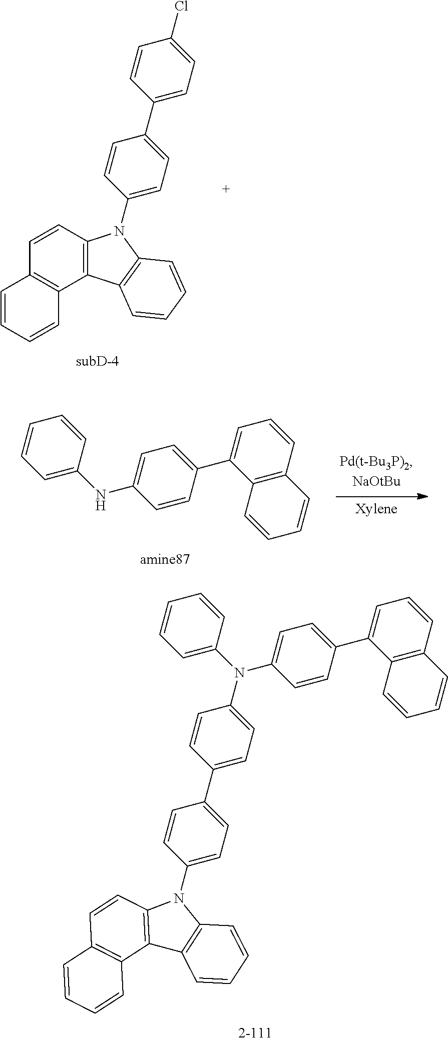 [SMILES: Clc1ccc(-c2ccc(-n3c4ccccc4c4c5ccccc5ccc43)cc2)cc1 ; c1ccc(Nc2ccc(-c3cccc4ccccc34)cc2)cc1 ; c1ccc(N(c2ccc(-c3ccc(-n4c5ccccc5c5c6ccccc6ccc54)cc3)cc2)c2ccc(-c3cccc4ccccc34)cc2)cc1]

Compound subD-4 (10 g, 24.8 mmol), Compound amine87 (7.7 g, 26 mmol) and sodium tert-butoxide (3.1 g, 32.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 11.8 g of Compound 2-111. (Yield: 72%, MS: [M+H]⁺=663)

### Synthesis Example 2-112

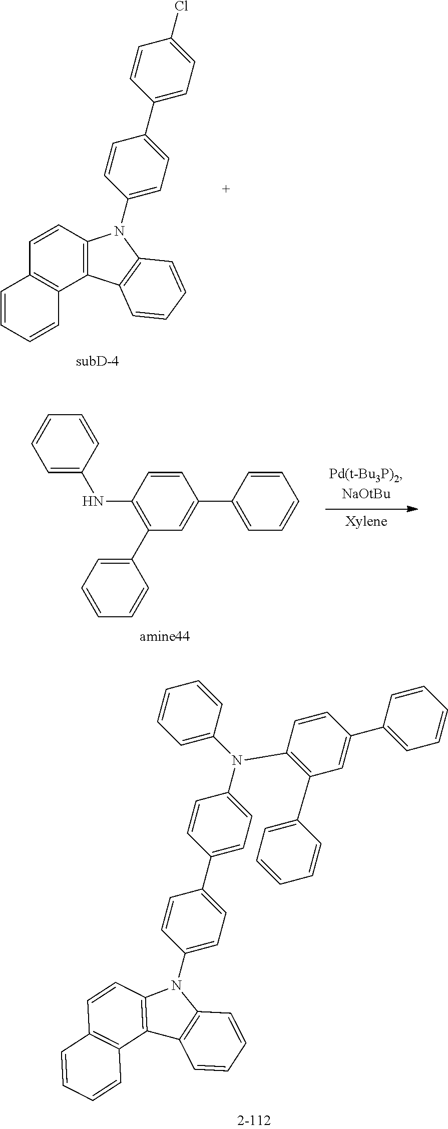 [SMILES: Clc1ccc(-c2ccc(-n3c4ccccc4c4c5ccccc5ccc43)cc2)cc1 ; c1ccc(Nc2ccc(-c3ccccc3)cc2-c2ccccc2)cc1 ; c1ccc(-c2ccc(N(c3ccccc3)c3ccc(-c4ccc(-n5c6ccccc6c6c7ccccc7ccc65)cc4)cc3)c(-c3ccccc3)c2)cc1]

Compound subD-4 (10 g, 24.8 mmol), Compound amine44 (8.4 g, 26 mmol) and sodium tert-butoxide (3.1 g, 32.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 12.8 g of Compound 2-112. (Yield: 75%, MS: [M+H]⁺=689)

### Synthesis Example 2-113

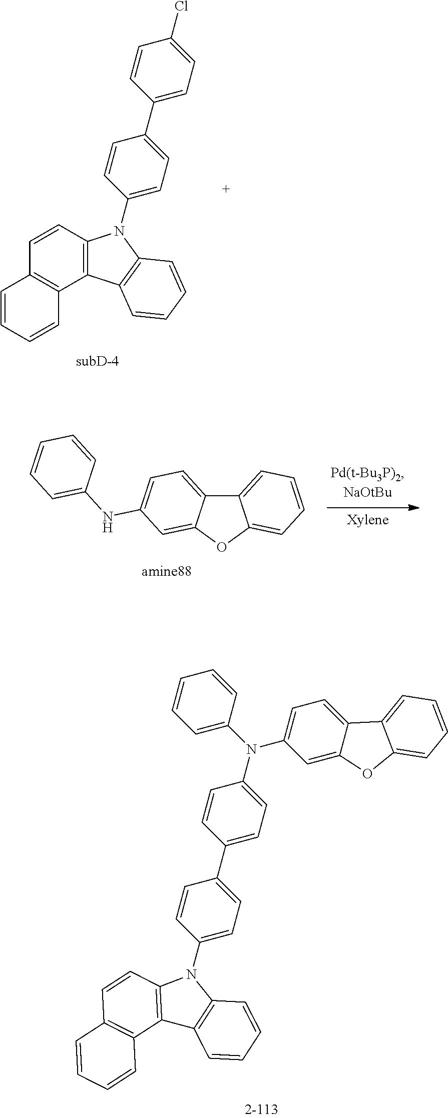 [SMILES: Clc1ccc(-c2ccc(-n3c4ccccc4c4c5ccccc5ccc43)cc2)cc1 ; c1ccc(Nc2ccc3c(c2)oc2ccccc23)cc1 ; c1ccc(N(c2ccc(-c3ccc(-n4c5ccccc5c5c6ccccc6ccc54)cc3)cc2)c2ccc3c(c2)oc2ccccc23)cc1]

Compound subD-4 (10 g, 24.8 mmol), Compound amine88 (6.7 g, 26 mmol) and sodium tert-butoxide (3.1 g, 32.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 10.1 g of Compound 2-113. (Yield: 65%, MS: [M+H]⁺=627)

### Synthesis Example 2-114

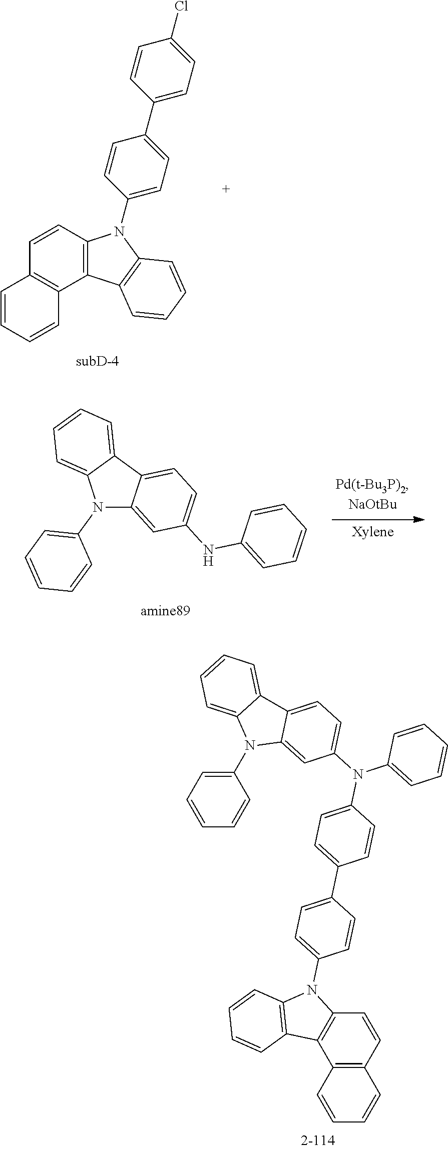 [SMILES: Clc1ccc(-c2ccc(-n3c4ccccc4c4c5ccccc5ccc43)cc2)cc1 ; c1ccc(Nc2ccc3c4ccccc4n(-c4ccccc4)c3c2)cc1 ; c1ccc(N(c2ccc(-c3ccc(-n4c5ccccc5c5c6ccccc6ccc54)cc3)cc2)c2ccc3c4ccccc4n(-c4ccccc4)c3c2)cc1]

Compound subD-4 (10 g, 24.8 mmol), Compound amine89 (8.7 g, 26 mmol) and sodium tert-butoxide (3.1 g, 32.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 11.3 g of Compound 2-114. (Yield: 65%, MS: [M+H]⁺=702)

### Synthesis Example 2-115

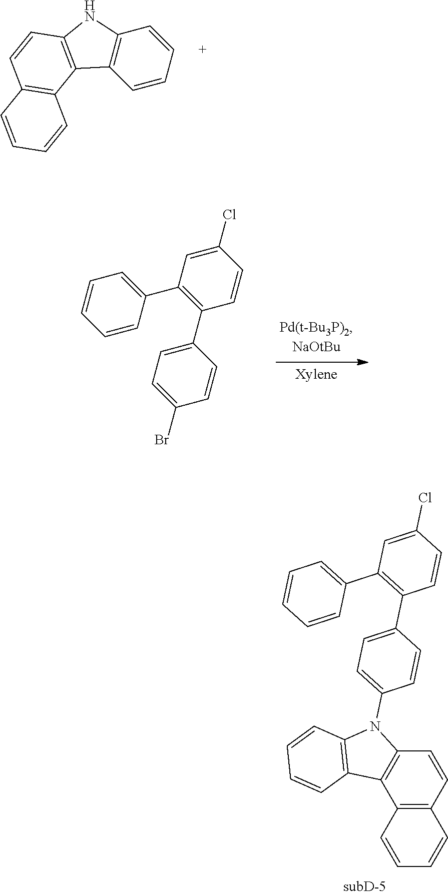 [SMILES: c1ccc2c(c1)ccc1[nH]c3ccccc3c12 ; Clc1ccc(-c2ccc(Br)cc2)c(-c2ccccc2)c1 ; Clc1ccc(-c2ccc(-n3c4ccccc4c4c5ccccc5ccc43)cc2)c(-c2ccccc2)c1]

7H-benzo[c]carbazole (10 g, 46 mmol), 4-bromo-4′-chloro-1,1′:2′,1″-terphenyl (16.6 g, 48.3 mmol) and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.2 g, 0.5 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 15.7 g of Compound subD-5. (Yield: 71%, MS: [M+H]⁺=480)

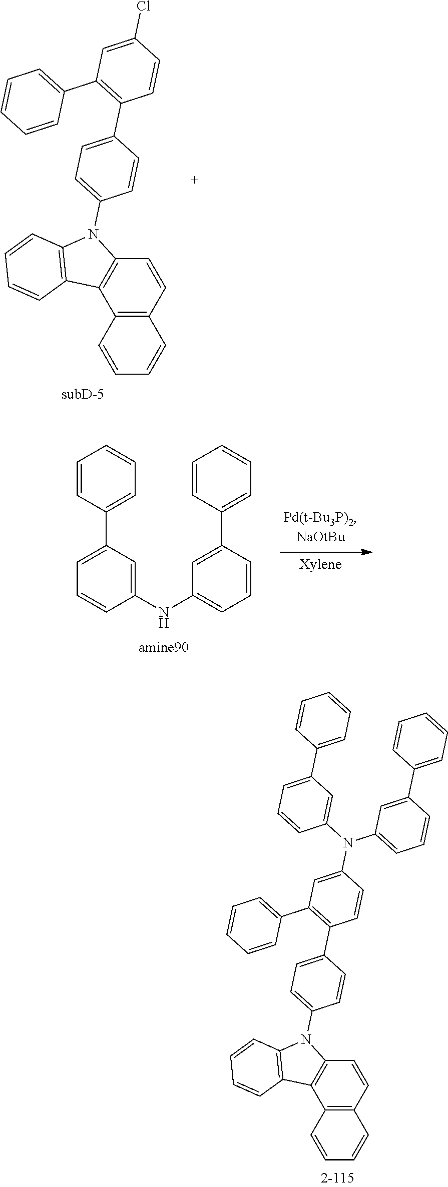 [SMILES: Clc1ccc(-c2ccc(-n3c4ccccc4c4c5ccccc5ccc43)cc2)c(-c2ccccc2)c1 ; c1ccc(-c2cccc(Nc3cccc(-c4ccccc4)c3)c2)cc1 ; c1ccc(-c2cccc(N(c3cccc(-c4ccccc4)c3)c3ccc(-c4ccc(-n5c6ccccc6c6c7ccccc7ccc65)cc4)c(-c4ccccc4)c3)c2)cc1]

Compound subD-5 (10 g, 20.8 mmol), Compound amine90 (7 g, 21.9 mmol) and sodium tert-butoxide (2.6 g, 27.1 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 11.1 g of Compound 2-115. (Yield: 70%, MS: [M+H]⁺=765)

### Synthesis Example 2-116

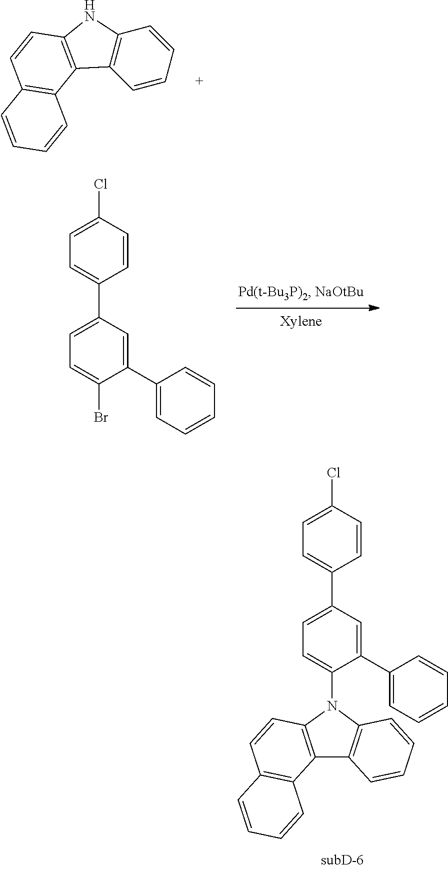 [SMILES: c1ccc2c(c1)ccc1[nH]c3ccccc3c12 ; Clc1ccc(-c2ccc(Br)c(-c3ccccc3)c2)cc1 ; Clc1ccc(-c2ccc(-n3c4ccccc4c4c5ccccc5ccc43)c(-c3ccccc3)c2)cc1]

7H-benzo[c]carbazole (10 g, 46 mmol), 4′-bromo-4-chloro-1,1′:3′,1″-terphenyl (16.6 g, 48.3 mmol) and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.2 g, 0.5 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 16.1 g of Compound subD-6. (Yield: 73%, MS: [M+H]⁺=480)

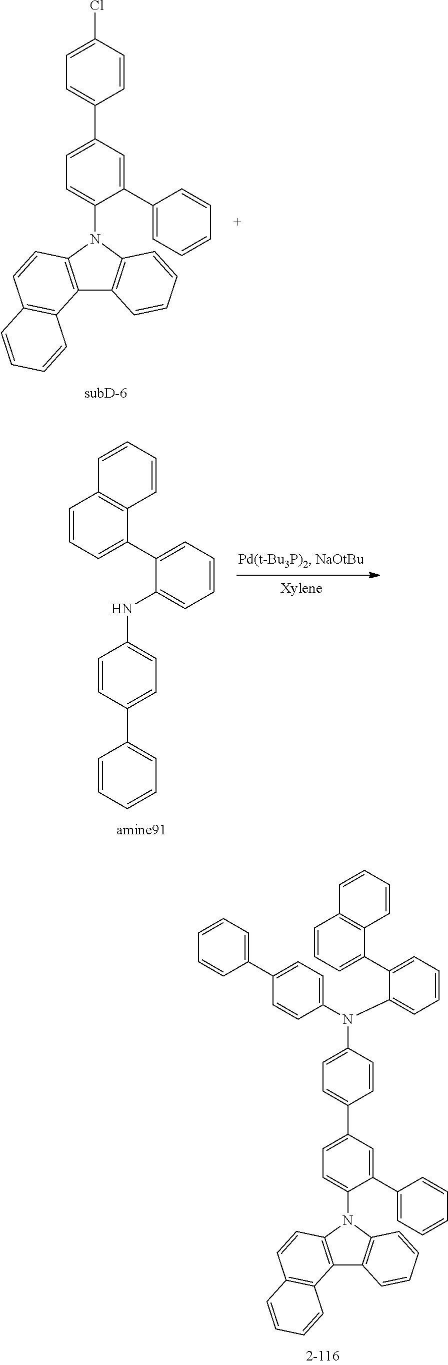 [SMILES: Clc1ccc(-c2ccc(-n3c4ccccc4c4c5ccccc5ccc43)c(-c3ccccc3)c2)cc1 ; c1ccc(-c2ccc(Nc3ccccc3-c3cccc4ccccc34)cc2)cc1 ; c1ccc(-c2ccc(N(c3ccc(-c4ccc(-n5c6ccccc6c6c7ccccc7ccc65)c(-c5ccccc5)c4)cc3)c3ccccc3-c3cccc4ccccc34)cc2)cc1]

Compound subD-6 (10 g, 20.8 mmol), Compound amine91 (8.1 g, 21.9 mmol) and sodium tert-butoxide (2.6 g, 27.1 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 10.9 g of Compound 2-116. (Yield: 64%, MS: [M+H]⁺=815)

### Synthesis Example 2-117

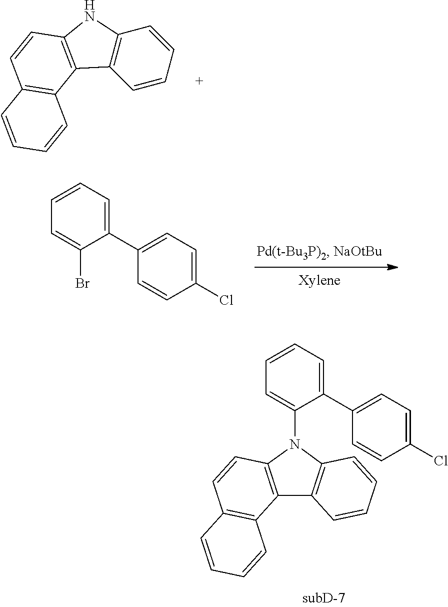

7H-benzo[c]carbazole (10 g, 46 mmol), 2-bromo-4′-chloro-1,1′-biphenyl (12.9 g, 48.3 mmol) and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.2 g, 0.5 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.4 g of Compound subD-7. (Yield: 72%, MS: [M+H]⁺=404)

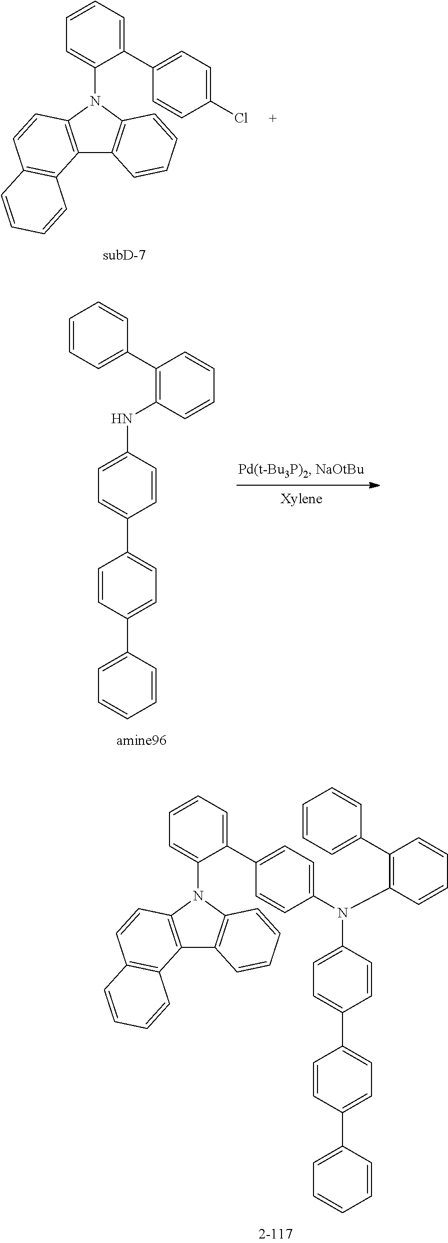 [SMILES: Clc1ccc(-c2ccccc2-n2c3ccccc3c3c4ccccc4ccc32)cc1 ; c1ccc(-c2ccc(-c3ccc(Nc4ccccc4-c4ccccc4)cc3)cc2)cc1 ; c1ccc(-c2ccc(-c3ccc(N(c4ccc(-c5ccccc5-n5c6ccccc6c6c7ccccc7ccc65)cc4)c4ccccc4-c4ccccc4)cc3)cc2)cc1]

Compound subD-7 (10 g, 24.8 mmol), Compound amine96 (10.3 g, 26 mmol) and sodium tert-butoxide (3.1 g, 32.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.6 g of Compound 2-117. (Yield: 72%, MS: [M+H]⁺=765)

### Synthesis Example 2-118

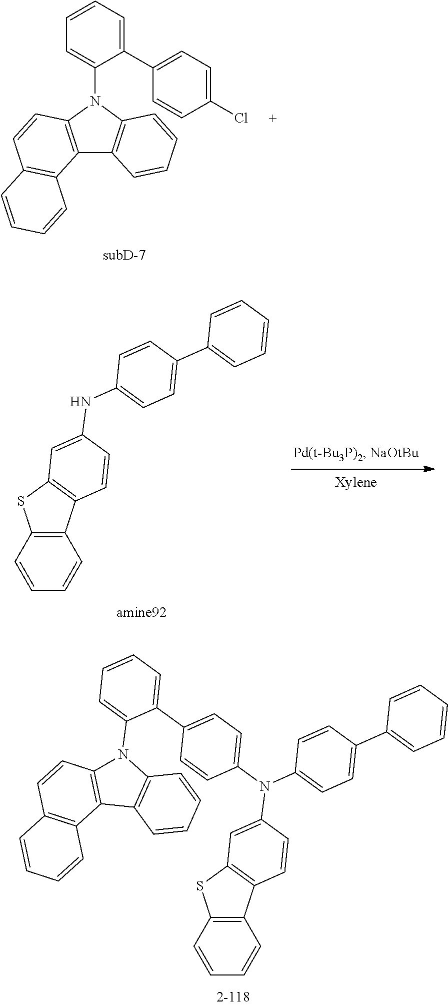 [SMILES: Clc1ccc(-c2ccccc2-n2c3ccccc3c3c4ccccc4ccc32)cc1 ; c1ccc(-c2ccc(Nc3ccc4c(c3)sc3ccccc34)cc2)cc1 ; c1ccc(-c2ccc(N(c3ccc(-c4ccccc4-n4c5ccccc5c5c6ccccc6ccc54)cc3)c3ccc4c(c3)sc3ccccc34)cc2)cc1]

Compound subD-7 (10 g, 24.8 mmol), Compound amine92 (9.1 g, 26 mmol) and sodium tert-butoxide (3.1 g, 32.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 12.1 g of Compound 2-118. (Yield: 68%, MS: [M+H]⁺=719)

### Synthesis Example 2-119

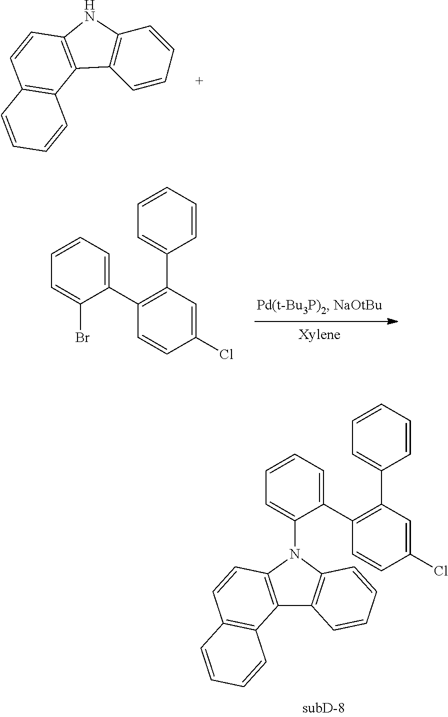 [SMILES: c1ccc2c(c1)ccc1[nH]c3ccccc3c12 ; Clc1ccc(-c2ccccc2Br)c(-c2ccccc2)c1 ; Clc1ccc(-c2ccccc2-n2c3ccccc3c3c4ccccc4ccc32)c(-c2ccccc2)c1]

7H-benzo[c]carbazole (10 g, 46 mmol), 2-bromo-4′-chloro-1,1′:2′,1″-terphenyl (16.6 g, 48.3 mmol) and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.2 g, 0.5 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 16.3 g f Compound subD-8. (Yield: 74%, MS: [M+H]⁺=480)

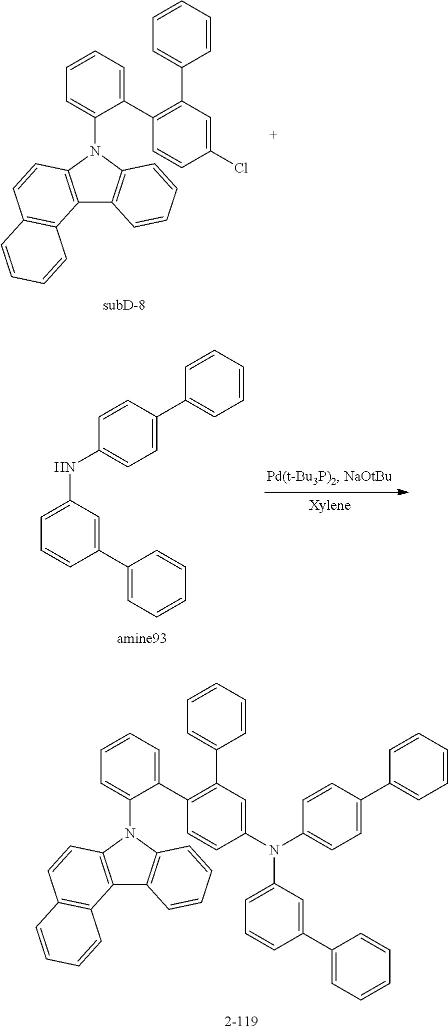 [SMILES: Clc1ccc(-c2ccccc2-n2c3ccccc3c3c4ccccc4ccc32)c(-c2ccccc2)c1 ; c1ccc(-c2ccc(Nc3cccc(-c4ccccc4)c3)cc2)cc1 ; c1ccc(-c2ccc(N(c3cccc(-c4ccccc4)c3)c3ccc(-c4ccccc4-n4c5ccccc5c5c6ccccc6ccc54)c(-c4ccccc4)c3)cc2)cc1]

Compound subD-8 (10 g, 20.8 mmol), Compound amine93 (7 g, 21.9 mmol) and sodium tert-butoxide (2.6 g, 27.1 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 10.5 g of Compound 2-119. (Yield: 66%, MS: [M+H]⁺=765)

### Synthesis Example 2-120

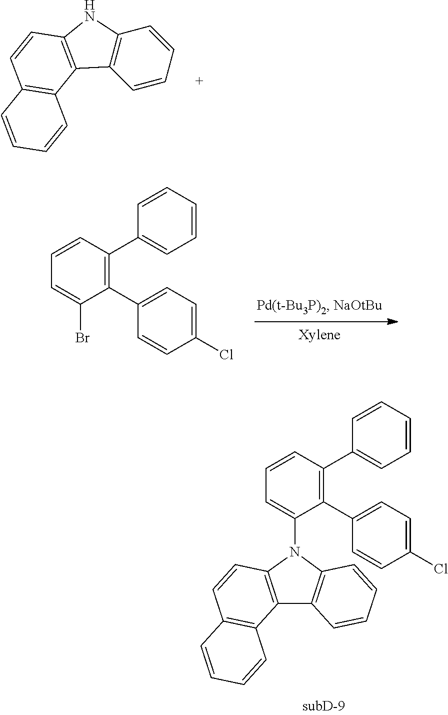 [SMILES: c1ccc2c(c1)ccc1[nH]c3ccccc3c12 ; Clc1ccc(-c2c(Br)cccc2-c2ccccc2)cc1 ; Clc1ccc(-c2c(-c3ccccc3)cccc2-n2c3ccccc3c3c4ccccc4ccc32)cc1]

7H-benzo[c]carbazole (10 g, 46 mmol), 3′-bromo-4″-chloro-1,1′:2′,1″-terphenyl (16.6 g, 48.3 mmol) and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.2 g, 0.5 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 15.4 g of Compound subD-9. (Yield: 70%, MS: [M+H]⁺=480)

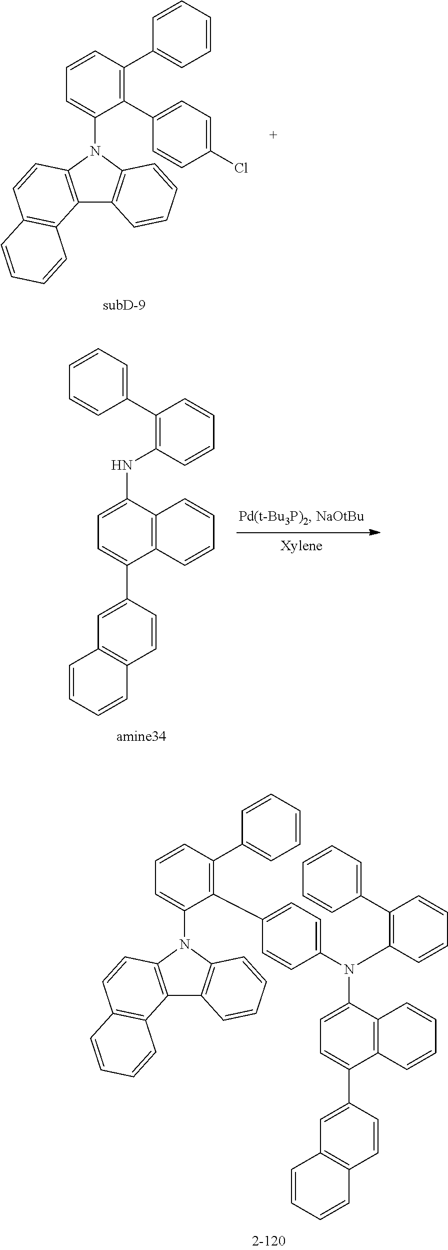 [SMILES: Clc1ccc(-c2c(-c3ccccc3)cccc2-n2c3ccccc3c3c4ccccc4ccc32)cc1 ; c1ccc(-c2ccccc2Nc2ccc(-c3ccc4ccccc4c3)c3ccccc23)cc1 ; c1ccc(-c2ccccc2N(c2ccc(-c3c(-c4ccccc4)cccc3-n3c4ccccc4c4c5ccccc5ccc43)cc2)c2ccc(-c3ccc4ccccc4c3)c3ccccc23)cc1]

Compound subD-9 (10 g, 20.8 mmol), Compound amine34 (9.2 g, 21.9 mmol) and sodium tert-butoxide (2.6 g, 27.1 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 11.9 g of Compound 2-120. (Yield: 66%, MS: [M+H]⁺=865)

### Synthesis Example 2-121

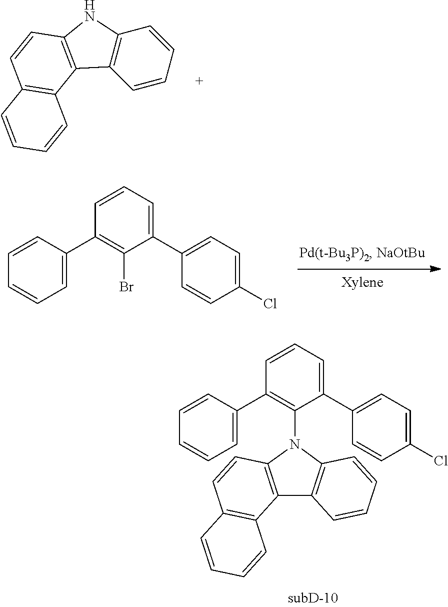 [SMILES: c1ccc2c(c1)ccc1[nH]c3ccccc3c12 ; Clc1ccc(-c2cccc(-c3ccccc3)c2Br)cc1 ; Clc1ccc(-c2cccc(-c3ccccc3)c2-n2c3ccccc3c3c4ccccc4ccc32)cc1]

7H-benzo[c]carbazole (10 g, 46 mmol), 2′-bromo-4-chloro-1,1′:3′,1″-terphenyl (16.6 g, 48.3 mmol) and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.2 g, 0.5 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 15.4 g of Compound subD-10. (Yield: 70%, MS: [M+H]⁺=480)

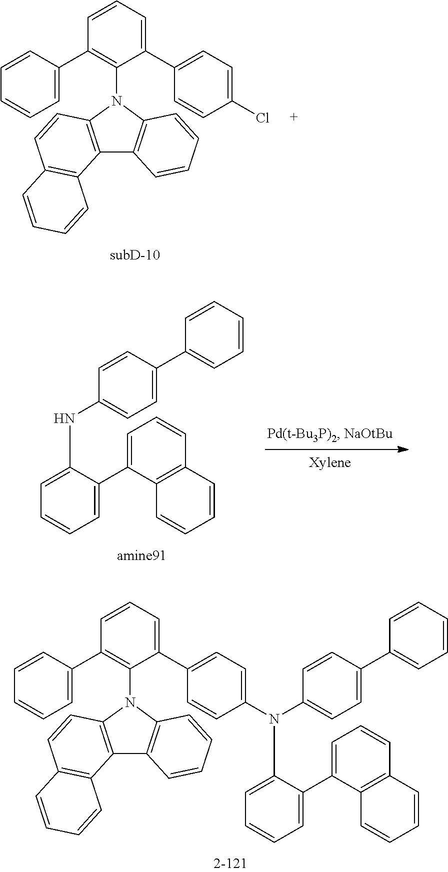 [SMILES: Clc1ccc(-c2cccc(-c3ccccc3)c2-n2c3ccccc3c3c4ccccc4ccc32)cc1 ; c1ccc(-c2ccc(Nc3ccccc3-c3cccc4ccccc34)cc2)cc1 ; c1ccc(-c2ccc(N(c3ccc(-c4cccc(-c5ccccc5)c4-n4c5ccccc5c5c6ccccc6ccc54)cc3)c3ccccc3-c3cccc4ccccc34)cc2)cc1]

Compound subD-10 (10 g, 20.8 mmol), Compound amine91 (8.1 g, 21.9 mmol) and sodium tert-butoxide (2.6 g, 27.1 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 11.9 g of Compound 2-121. (Yield: 70%, MS: [M+H]⁺=815)

### Synthesis Example 2-122

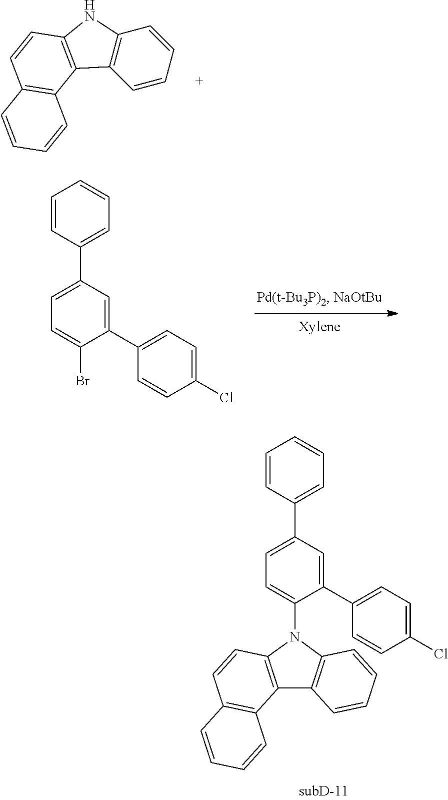

7H-benzo[c]carbazole (10 g, 46 mmol), 6′-bromo-4-chloro-1,1′:3′,1″-terphenyl (16.6 g, 48.3 mmol) and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.2 g, 0.5 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 15.4 g of Compound subD-11. (Yield: 70%, MS: [M+H]⁺=480)

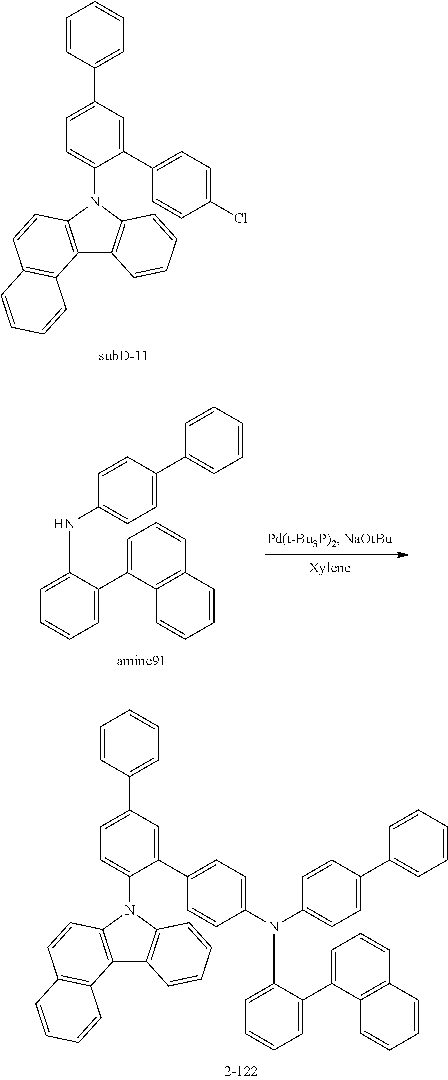 [SMILES: Clc1ccc(-c2cc(-c3ccccc3)ccc2-n2c3ccccc3c3c4ccccc4ccc32)cc1 ; c1ccc(-c2ccc(Nc3ccccc3-c3cccc4ccccc34)cc2)cc1 ; c1ccc(-c2ccc(N(c3ccc(-c4cc(-c5ccccc5)ccc4-n4c5ccccc5c5c6ccccc6ccc54)cc3)c3ccccc3-c3cccc4ccccc34)cc2)cc1]

Compound subD-11 (10 g, 20.8 mmol), Compound amine91 (8.1 g, 21.9 mmol) and sodium tert-butoxide (2.6 g, 27.1 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 12.6 g of Compound 2-122. (Yield: 74%, MS: [M+H]⁺=815)

### Synthesis Example 2-123

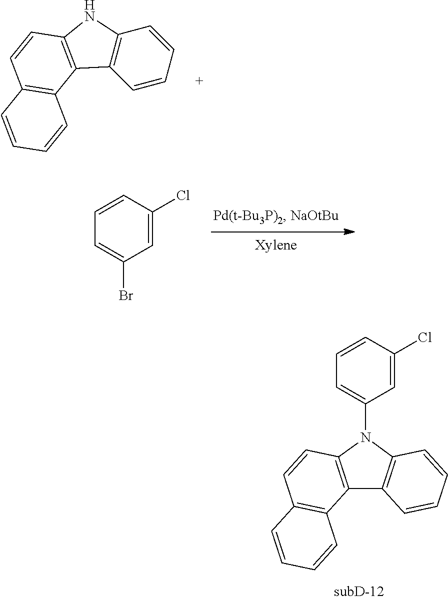 [SMILES: c1ccc2c(c1)ccc1[nH]c3ccccc3c12 ; Clc1cccc(Br)c1 ; Clc1cccc(-n2c3ccccc3c3c4ccccc4ccc32)c1]

7H-benzo[c]carbazole (10 g, 46 mmol), 1-bromo-3-chlorobenzene (9.3 g, 48.3 mmol) and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.2 g, 0.5 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 10.7 g of Compound subD-12. (Yield: 71%, MS: [M+H]⁺=328)

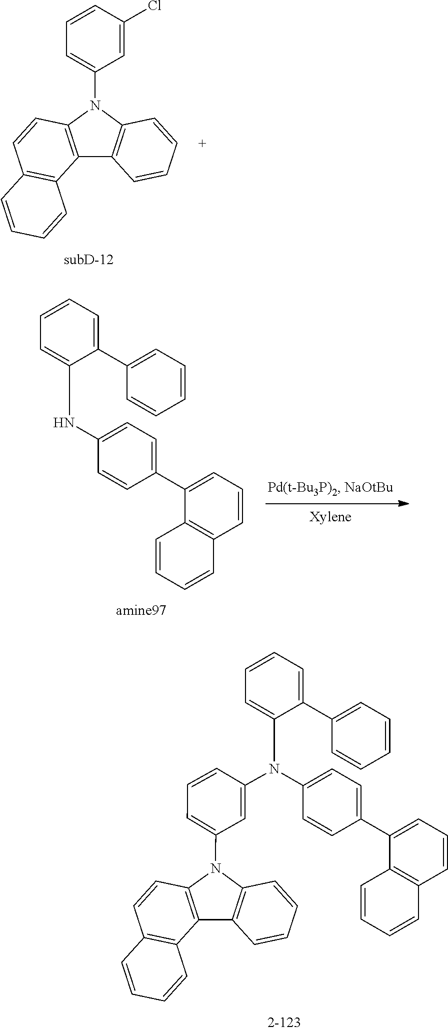 [SMILES: Clc1cccc(-n2c3ccccc3c3c4ccccc4ccc32)c1 ; c1ccc(-c2ccccc2Nc2ccc(-c3cccc4ccccc34)cc2)cc1 ; c1ccc(-c2ccccc2N(c2ccc(-c3cccc4ccccc34)cc2)c2cccc(-n3c4ccccc4c4c5ccccc5ccc43)c2)cc1]

Compound subD-12 (10 g, 30.5 mmol), Compound amine97 (11.9 g, 32 mmol) and sodium tert-butoxide (3.8 g, 39.7 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.5 g of Compound 2-123. (Yield: 67%, MS: [M+H]⁺=663)

### Synthesis Example 2-124

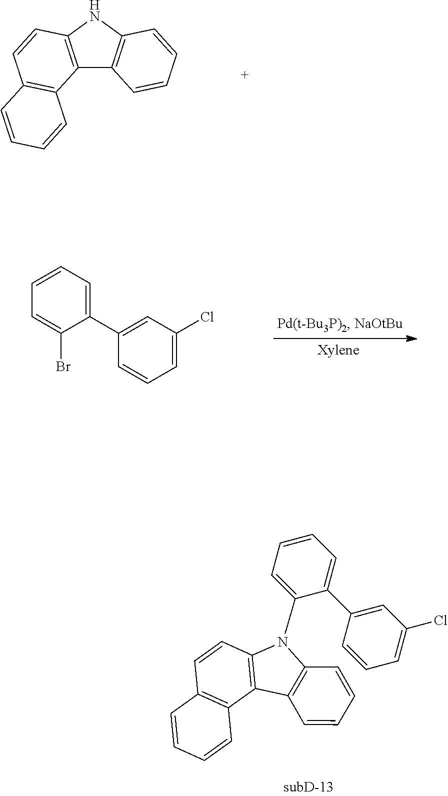 [SMILES: c1ccc2c(c1)ccc1[nH]c3ccccc3c12 ; Clc1cccc(-c2ccccc2Br)c1 ; Clc1cccc(-c2ccccc2-n2c3ccccc3c3c4ccccc4ccc32)c1]

7H-benzo[c]carbazole (10 g, 46 mmol), 2-bromo-3′-chloro-1,1′-biphenyl (12.9 g, 48.3 mmol) and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.2 g, 0.5 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to

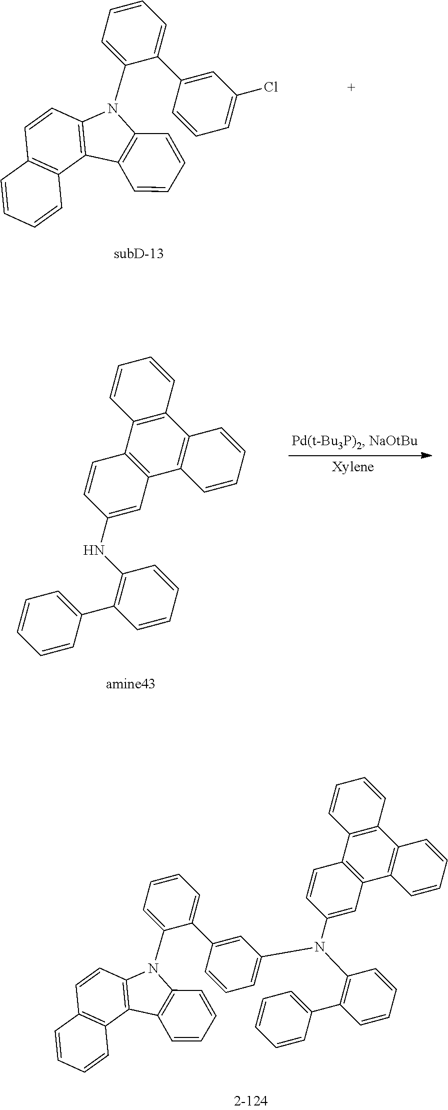 [SMILES: Clc1cccc(-c2ccccc2-n2c3ccccc3c3c4ccccc4ccc32)c1 ; c1ccc(-c2ccccc2Nc2ccc3c4ccccc4c4ccccc4c3c2)cc1 ; c1ccc(-c2ccccc2N(c2cccc(-c3ccccc3-n3c4ccccc4c4c5ccccc5ccc43)c2)c2ccc3c4ccccc4c4ccccc4c3c2)cc1]

prepare 12.6 go f Compound subD-13. (Yield: 68%, MS: [M+H]⁺=404)

Compound subD-13 (10 g, 24.8 mmol), Compound amine43 (10.3 g, 26 mmol) and sodium tert-butoxide (3.1 g, 32.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.2 g of Compound 2-124. (Yield: 70%, MS: [M+H]⁺=763)

### Synthesis Example 2-125

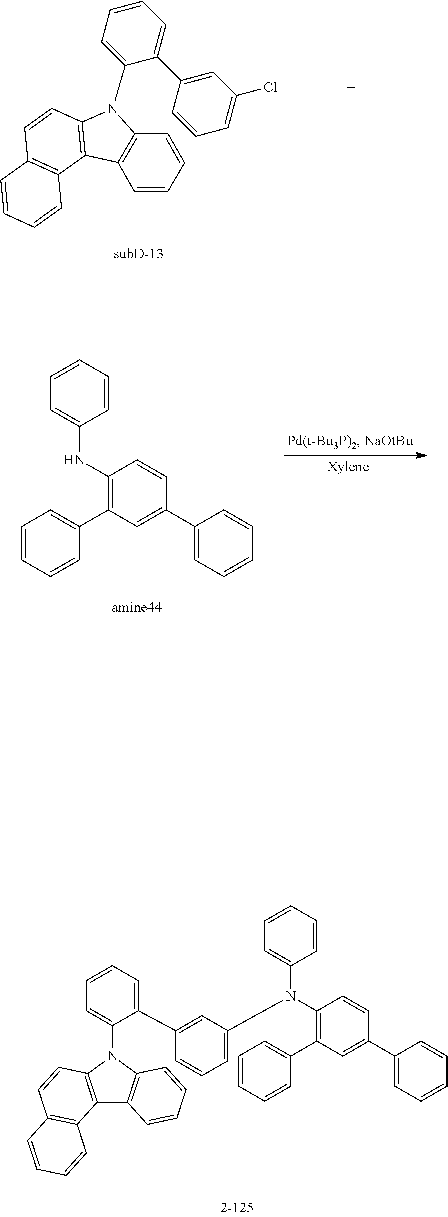 [SMILES: Clc1cccc(-c2ccccc2-n2c3ccccc3c3c4ccccc4ccc32)c1 ; c1ccc(Nc2ccc(-c3ccccc3)cc2-c2ccccc2)cc1 ; c1ccc(-c2ccc(N(c3ccccc3)c3cccc(-c4ccccc4-n4c5ccccc5c5c6ccccc6ccc54)c3)c(-c3ccccc3)c2)cc1]

Compound subD-13 (10 g, 24.8 mmol), Compound amine44 (8.4 g, 26 mmol) and sodium tert-butoxide (3.1 g, 32.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 11.4 g of Compound 2-125. (Yield: 67%, MS: [M+H]⁺=689)

### Synthesis Example 2-126

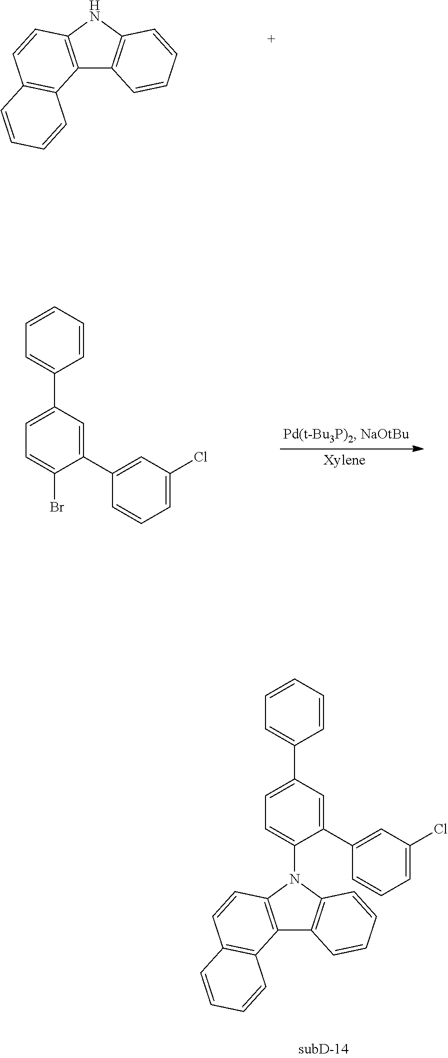 [SMILES: c1ccc2c(c1)ccc1[nH]c3ccccc3c12 ; Clc1cccc(-c2cc(-c3ccccc3)ccc2Br)c1 ; Clc1cccc(-c2cc(-c3ccccc3)ccc2-n2c3ccccc3c3c4ccccc4ccc32)c1]

7H-benzo[c]carbazole (10 g, 46 mmol), 6′-bromo-3-chloro-1,1′:3′,1″-terphenyl (16.6 g, 48.3 mmol) and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.2 g, 0.5 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 14.3 g of Compound subD-14. (Yield: 65%, MS: [M+H]⁺=480)

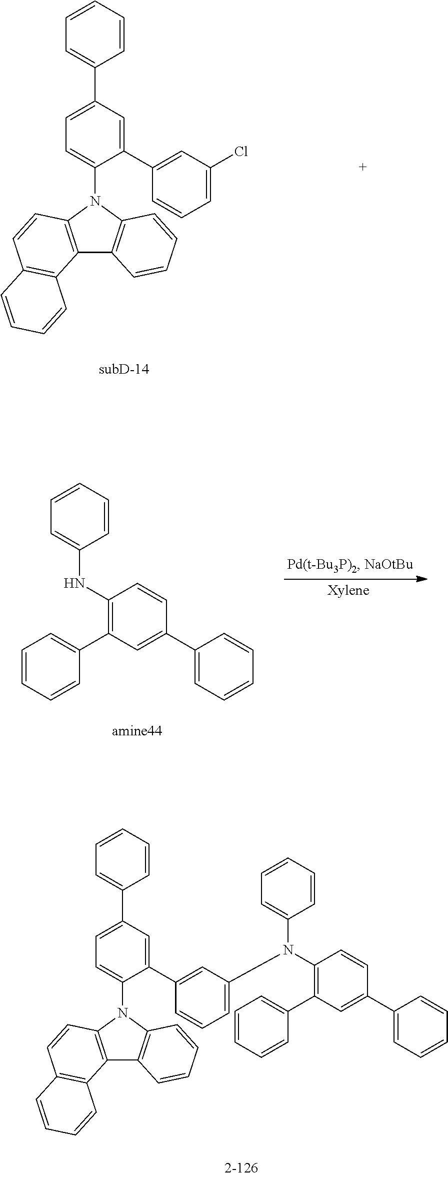 [SMILES: Clc1cccc(-c2cc(-c3ccccc3)ccc2-n2c3ccccc3c3c4ccccc4ccc32)c1 ; c1ccc(Nc2ccc(-c3ccccc3)cc2-c2ccccc2)cc1 ; c1ccc(-c2ccc(N(c3ccccc3)c3cccc(-c4cc(-c5ccccc5)ccc4-n4c5ccccc5c5c6ccccc6ccc54)c3)c(-c3ccccc3)c2)cc1]

Compound subD-14 (10 g, 20.8 mmol), Compound amine44 (7 g, 21.9 mmol) and sodium tert-butoxide (2.6 g, 27.1 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 10.2 g of Compound 2-126. (Yield: 64%, MS: [M+H]⁺=765)

### Synthesis Example 2-127

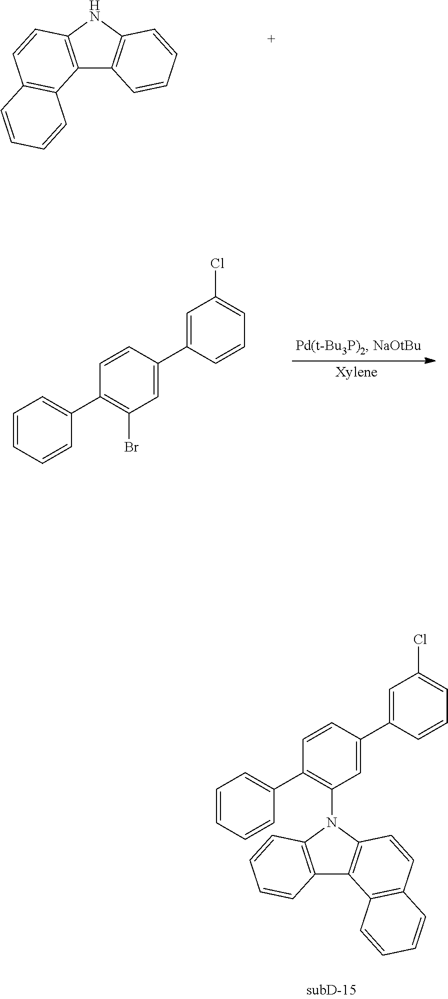 [SMILES: c1ccc2c(c1)ccc1[nH]c3ccccc3c12 ; Clc1cccc(-c2ccc(-c3ccccc3)c(Br)c2)c1 ; Clc1cccc(-c2ccc(-c3ccccc3)c(-n3c4ccccc4c4c5ccccc5ccc43)c2)c1]

7H-benzo[c]carbazole (10 g, 46 mmol), 2′-bromo-3″-chloro-1,1′:4′,1″-terphenyl (16.6 g, 48.3 mmol) and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.2 g, 0.5 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 15.9 g of Compound subD-15. (Yield: 72%, MS: [M+H]⁺=480)

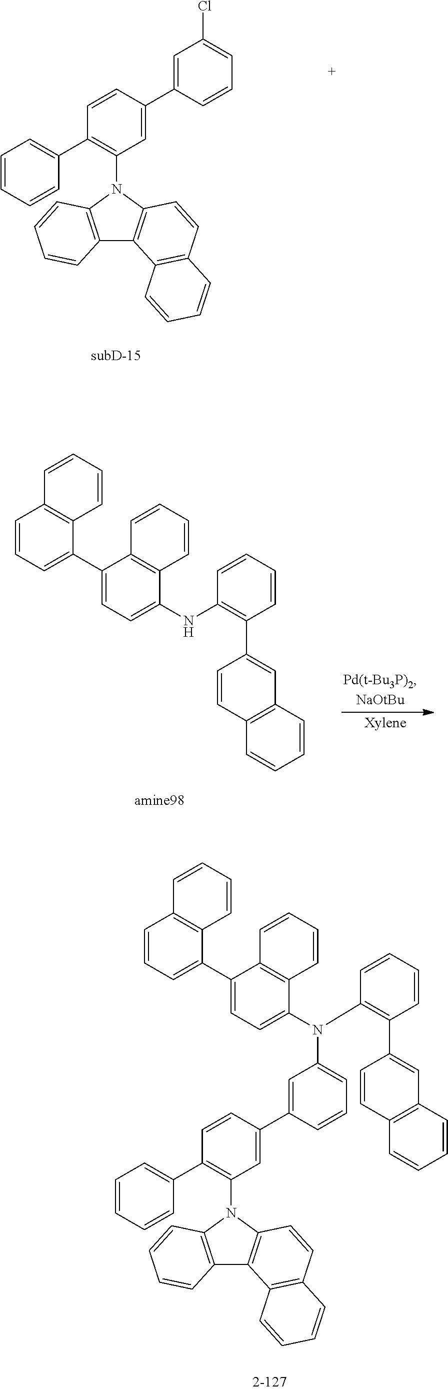 [SMILES: Clc1cccc(-c2ccc(-c3ccccc3)c(-n3c4ccccc4c4c5ccccc5ccc43)c2)c1 ; c1ccc(-c2ccc3ccccc3c2)c(Nc2ccc(-c3cccc4ccccc34)c3ccccc23)c1 ; c1ccc(-c2ccc(-c3cccc(N(c4ccccc4-c4ccc5ccccc5c4)c4ccc(-c5cccc6ccccc56)c5ccccc45)c3)cc2-n2c3ccccc3c3c4ccccc4ccc32)cc1]

Compound subD-15 (10 g, 20.8 mmol), Compound amine98 (10.3 g, 21.9 mmol) and sodium tert-butoxide (2.6 g, 27.1 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.1 g of Compound 2-127. (Yield: 69%, MS: [M+H]⁺=915)

### Synthesis Example 2-128

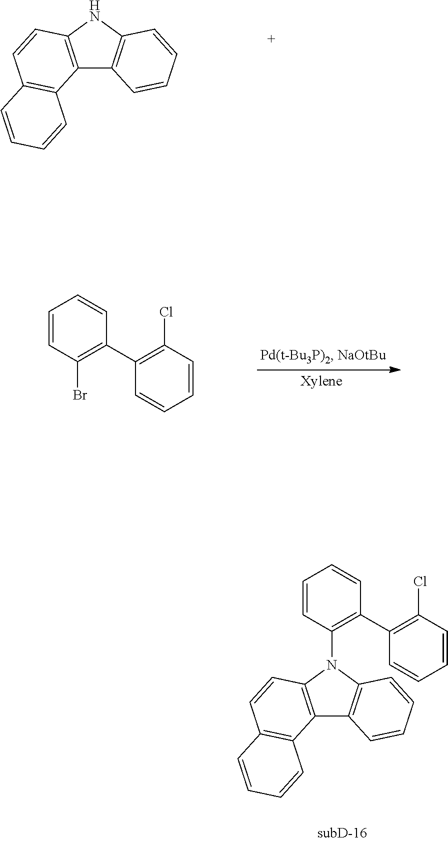 [SMILES: c1ccc2c(c1)ccc1[nH]c3ccccc3c12 ; Clc1ccccc1-c1ccccc1Br ; Clc1ccccc1-c1ccccc1-n1c2ccccc2c2c3ccccc3ccc21]

7H-benzo[c]carbazole (10 g, 46 mmol), 2-bromo-2′-chloro-1,1′-biphenyl (12.9 g, 48.3 mmol) and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.2 g, 0.5 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 11.7 g of Compound subD-16. (Yield: 63%, MS: [M+H]⁺=404)

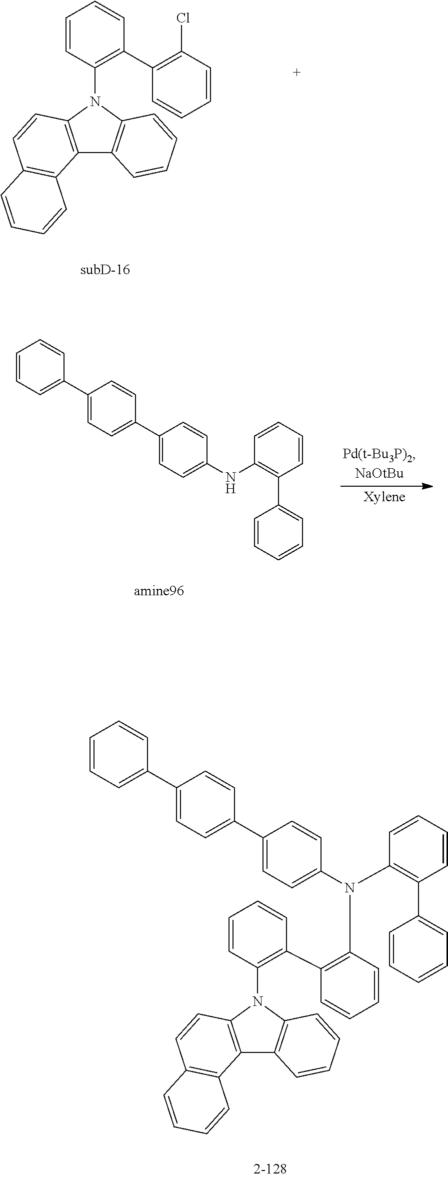 [SMILES: Clc1ccccc1-c1ccccc1-n1c2ccccc2c2c3ccccc3ccc21 ; c1ccc(-c2ccc(-c3ccc(Nc4ccccc4-c4ccccc4)cc3)cc2)cc1 ; c1ccc(-c2ccc(-c3ccc(N(c4ccccc4-c4ccccc4)c4ccccc4-c4ccccc4-n4c5ccccc5c5c6ccccc6ccc54)cc3)cc2)cc1]

Compound subD-16 (10 g, 24.8 mmol), Compound amine96 (10.3 g, 26 mmol) and sodium tert-butoxide (3.1 g, 32.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 14 g of Compound 2-128. (Yield: 74%, MS: [M+H]⁺=765)

### Synthesis Example 2-129

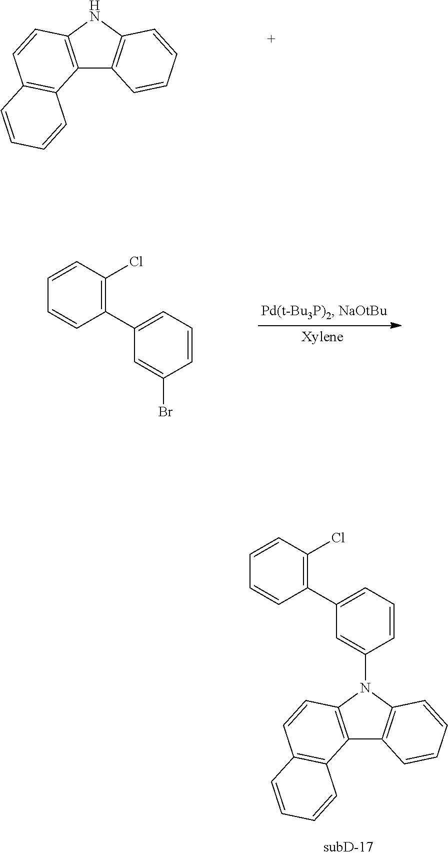 [SMILES: c1ccc2c(c1)ccc1[nH]c3ccccc3c12 ; Clc1ccccc1-c1cccc(Br)c1 ; Clc1ccccc1-c1cccc(-n2c3ccccc3c3c4ccccc4ccc32)c1]

7H-benzo[c]carbazole (10 g, 46 mmol), 3′-bromo-2-chloro-1,1′-biphenyl (12.9 g, 48.3 mmol) and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.2 g, 0.5 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13 g of Compound subD-17. (Yield: 70%, MS: [M+H]⁺=404)

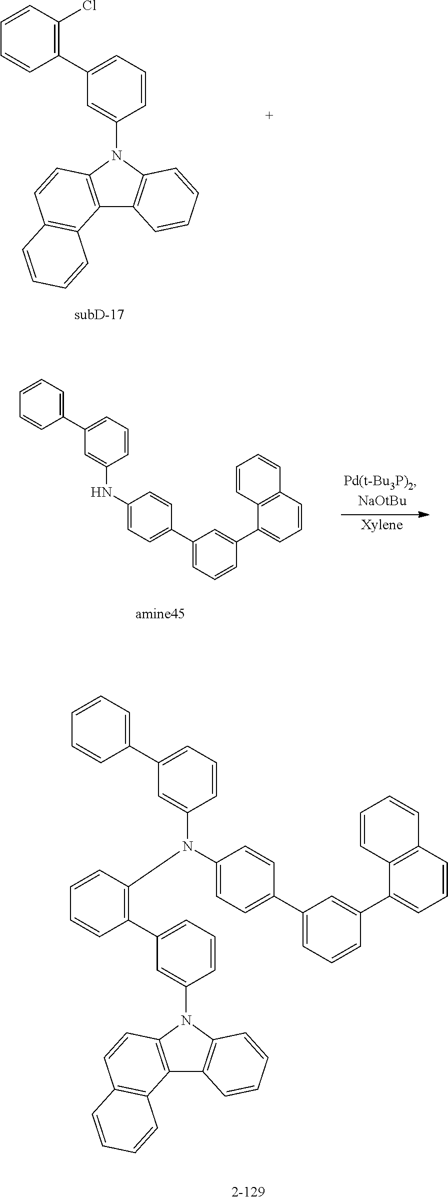 [SMILES: Clc1ccccc1-c1cccc(-n2c3ccccc3c3c4ccccc4ccc32)c1 ; c1ccc(-c2cccc(Nc3ccc(-c4cccc(-c5cccc6ccccc56)c4)cc3)c2)cc1 ; c1ccc(-c2cccc(N(c3ccc(-c4cccc(-c5cccc6ccccc56)c4)cc3)c3ccccc3-c3cccc(-n4c5ccccc5c5c6ccccc6ccc54)c3)c2)cc1]

Compound subD-17 (10 g, 24.8 mmol), Compound amine45 (11.6 g, 26 mmol) and sodium tert-butoxide (3.1 g, 32.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.9 g of Compound 2-129. (Yield: 69%, MS: [M+H]⁺=815)

### Synthesis Example 2-130

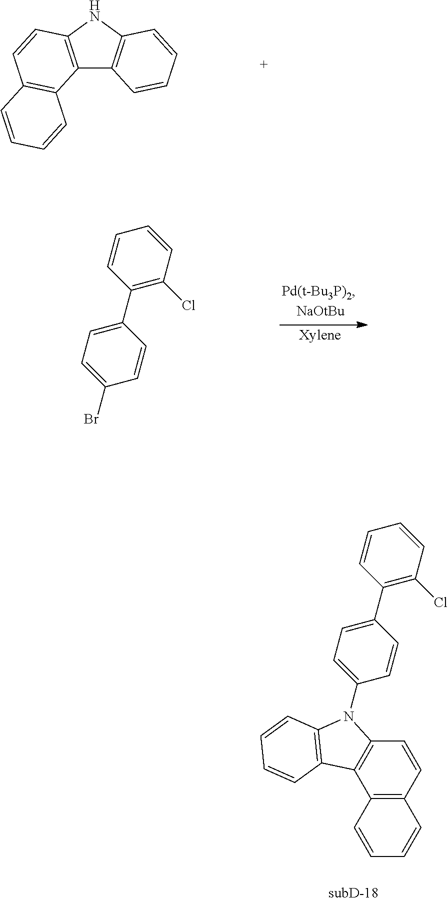 [SMILES: c1ccc2c(c1)ccc1[nH]c3ccccc3c12 ; Clc1ccccc1-c1ccc(Br)cc1 ; Clc1ccccc1-c1ccc(-n2c3ccccc3c3c4ccccc4ccc32)cc1]

7H-benzo[c]carbazole (10 g, 46 mmol), 4′-bromo-2-chloro-1,1′-biphenyl (12.9 g, 48.3 mmol) and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.2 g, 0.5 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 12.2 g of Compound subD-18. (Yield: 66%, MS: [M+H]⁺=404)

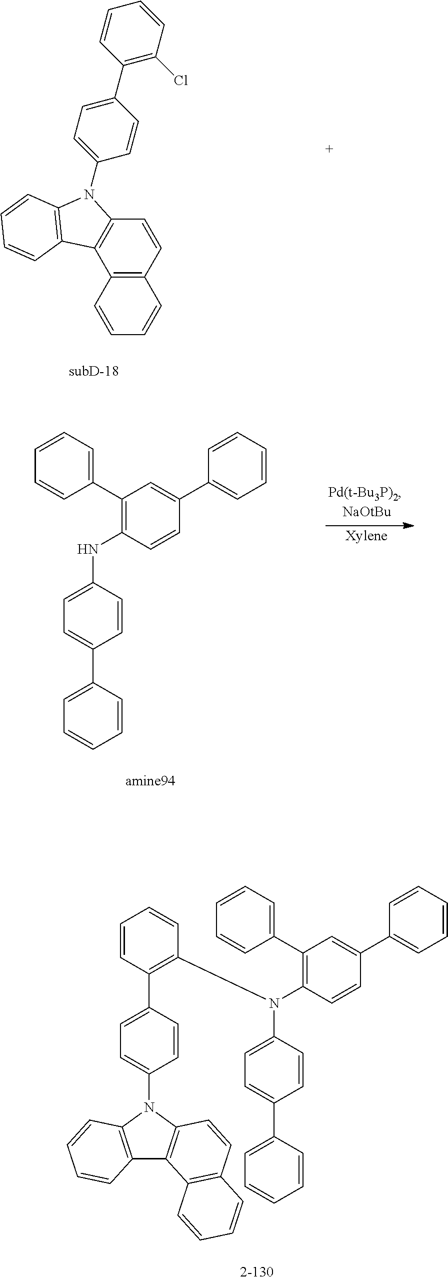 [SMILES: Clc1ccccc1-c1ccc(-n2c3ccccc3c3c4ccccc4ccc32)cc1 ; c1ccc(-c2ccc(Nc3ccc(-c4ccccc4)cc3-c3ccccc3)cc2)cc1 ; c1ccc(-c2ccc(N(c3ccccc3-c3ccc(-n4c5ccccc5c5c6ccccc6ccc54)cc3)c3ccc(-c4ccccc4)cc3-c3ccccc3)cc2)cc1]

Compound subD-18 (10 g, 24.8 mmol), Compound amine94 (10.3 g, 26 mmol) and sodium tert-butoxide (3.1 g, 32.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 11.5 g of Compound 2-130. (Yield: 61%, MS: [M+H]⁺=765)

### Example 1

A glass substrate on which a thin film of ITO (indium tin oxide) was coated in a thickness of 1000 Å was put into distilled water containing the detergent dissolved therein and washed by the ultrasonic wave. In this case, the used detergent was a product commercially available from Fisher Co. and the distilled water was one which had been twice filtered by using a filter commercially available from Millipore Co. The ITO was washed for 30 minutes, and ultrasonic washing was then repeated twice for 10 minutes by using distilled water. After the washing with distilled water was completed, the substrate was ultrasonically washed with isopropyl alcohol, acetone, and methanol solvent, and dried, after which it was transported to a plasma cleaner. Then, the substrate was cleaned with oxygen plasma for 5 minutes, and then transferred to a vacuum evaporator.

On the ITO transparent electrode thus prepared, the following Compound HI-1 was formed in a thickness of 1150 Å as a hole injection layer, but the following Compound A-1 was p-doped at a concentration of 1.5 wt. %. The following Compound HT-1 was vacuum deposited on the hole injection layer to form a hole transport layer with a film thickness of 800 Å. Then, the following Compound EB-1 was vacuum deposited on the hole transport layer to a film thickness of 150 Å to form an electron blocking layer. Then, the previously prepared Compound 1-1, Compound 2-1, and the following Compound Dp-7 were vacuum-deposited in a weight ratio of 49:49:2 on the EB-1 deposited film to form a red light emitting layer with a film thickness of 400 Å. The following Compound HB-1 was vacuum deposited on the light emitting layer to a film thickness of 30 Å to form a hole blocking layer. Then, the following Compound ET-1 and the following Compound LiQ were vacuum deposited in a weight ratio of 2:1 on the hole blocking layer to form an electron injection and transport layer with a film thickness of 300 Å. Lithium fluoride (LiF) and aluminum were sequentially deposited to have a thickness of 12 Å and 1,000 Å, respectively, on the electron injection and transport layer, thereby forming a cathode.

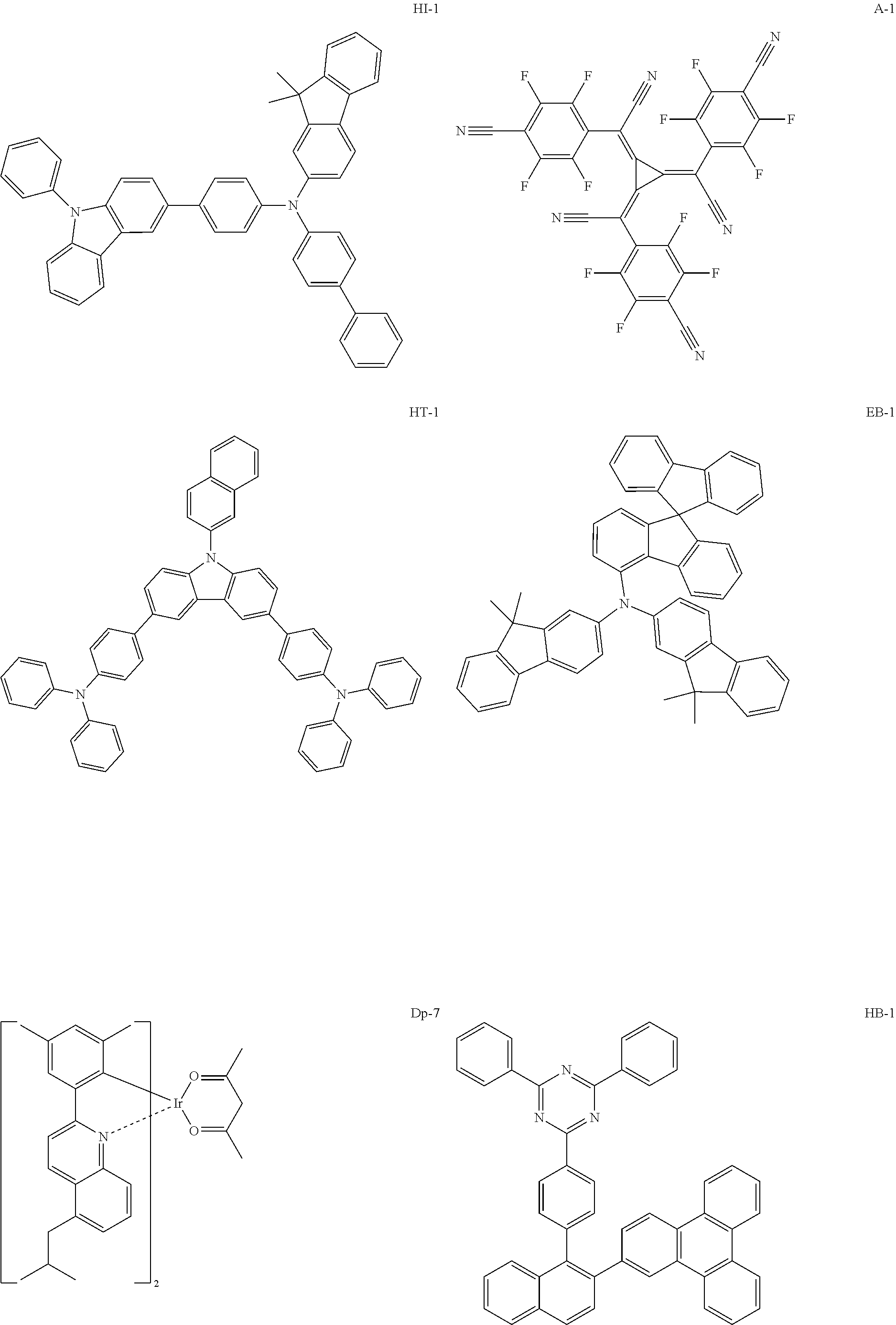

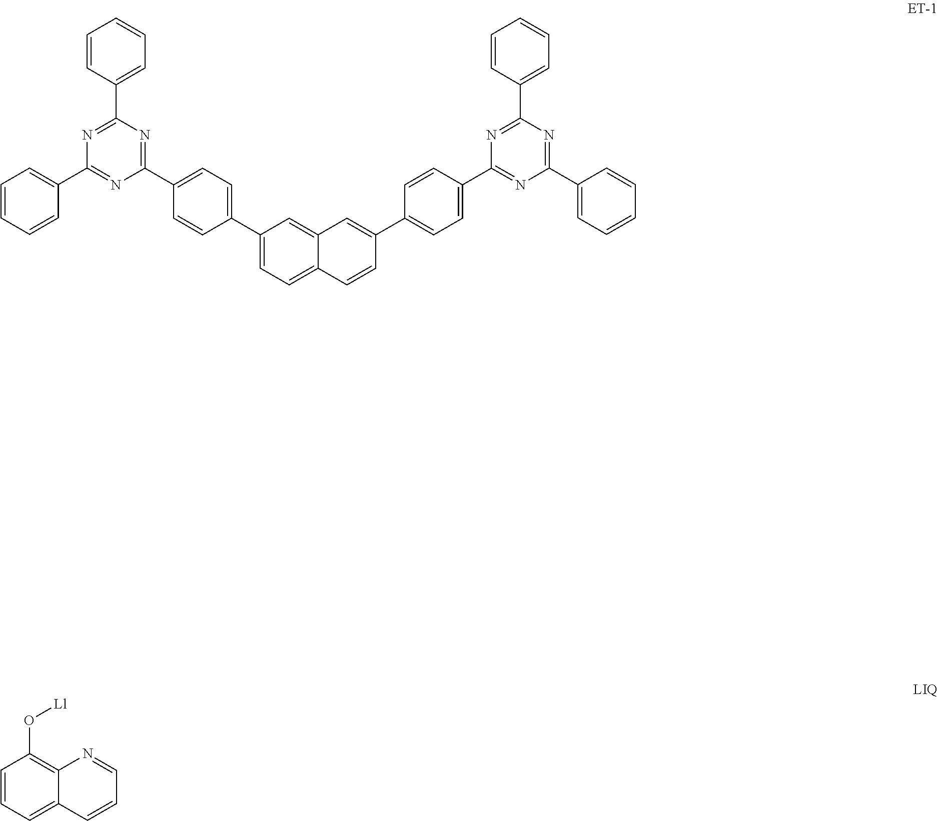

In the above-mentioned processes, the deposition rates of the organic materials were maintained at 0.4˜0.7 Å/sec, the deposition rates of lithium fluoride and the aluminum of the cathode were maintained at 0.3 Å/sec and 2 Å/sec, respectively, and the degree of vacuum during the deposition was maintained at 2×10⁻⁷˜5×10⁻⁶ torr, thereby manufacturing an organic light emitting device.

### Examples 2 to 340

An organic light emitting device was manufactured in the same manner as in Example 1, except that in the organic light emitting device of Example 1, the compound represented by Chemical Formula 1 and the compound represented by Chemical Formula 2 shown in Table 1 below were co-deposited and used in a weight ratio of 1:1 instead of Compound 1-1 and Compound 2-1 as the first host and second host.

### Comparative Examples 1 to 60

An organic light emitting device was manufactured in the same manner as in Example 1, except that in the organic light emitting device of Example 1, the following Comparative Compounds A-1 to A-12 were used instead of Compound 1-1 as the first host and the compound represented by Chemical Formula 2 shown in Table 2 below was used instead of Compound 2-1 as a second host, wherein these two host compounds were co-deposited and used in a weight ratio of 1:1. The specific structures of the Compounds A-1 to A-12 are as follows.

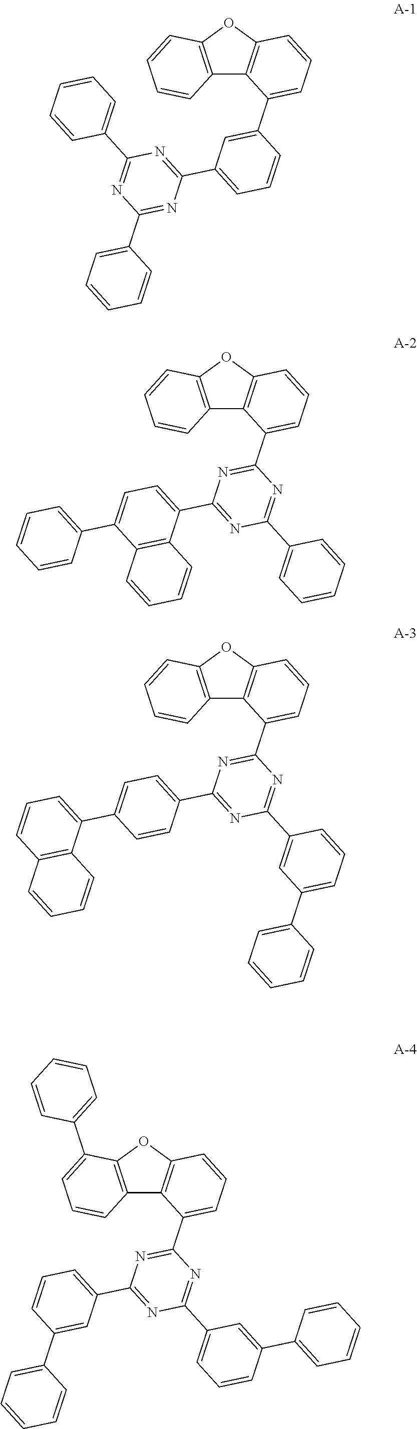 [SMILES: c1ccc(-c2nc(-c3ccccc3)nc(-c3cccc(-c4cccc5oc6ccccc6c45)c3)n2)cc1 ; c1ccc(-c2nc(-c3ccc(-c4ccccc4)c4ccccc34)nc(-c3cccc4oc5ccccc5c34)n2)cc1 ; c1ccc(-c2cccc(-c3nc(-c4ccc(-c5cccc6ccccc56)cc4)nc(-c4cccc5oc6ccccc6c45)n3)c2)cc1 ; c1ccc(-c2cccc(-c3nc(-c4cccc(-c5ccccc5)c4)nc(-c4cccc5oc6c(-c7ccccc7)cccc6c45)n3)c2)cc1]

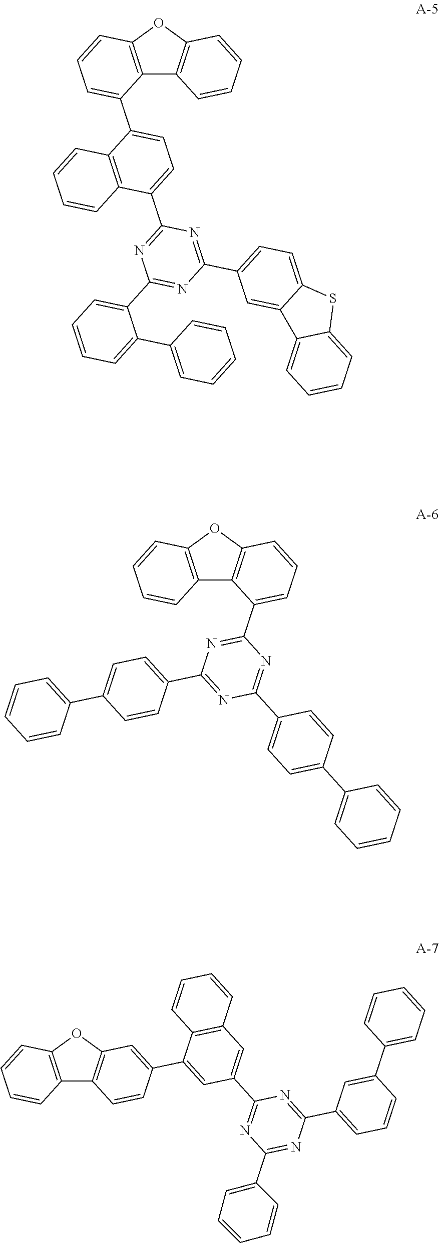 [SMILES: c1ccc(-c2ccccc2-c2nc(-c3ccc4sc5ccccc5c4c3)nc(-c3ccc(-c4cccc5oc6ccccc6c45)c4ccccc34)n2)cc1 ; c1ccc(-c2ccc(-c3nc(-c4ccc(-c5ccccc5)cc4)nc(-c4cccc5oc6ccccc6c45)n3)cc2)cc1 ; c1ccc(-c2cccc(-c3nc(-c4ccccc4)nc(-c4cc(-c5ccc6c(c5)oc5ccccc56)c5ccccc5c4)n3)c2)cc1]

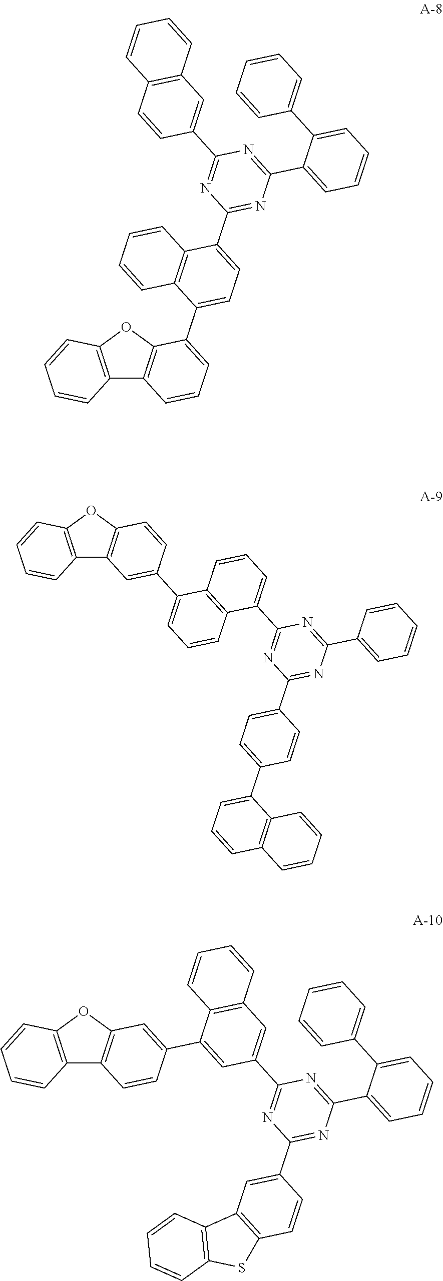 [SMILES: c1ccc(-c2ccccc2-c2nc(-c3ccc4ccccc4c3)nc(-c3ccc(-c4cccc5c4oc4ccccc45)c4ccccc34)n2)cc1 ; c1ccc(-c2nc(-c3ccc(-c4cccc5ccccc45)cc3)nc(-c3cccc4c(-c5ccc6oc7ccccc7c6c5)cccc34)n2)cc1 ; c1ccc(-c2ccccc2-c2nc(-c3cc(-c4ccc5c(c4)oc4ccccc45)c4ccccc4c3)nc(-c3ccc4sc5ccccc5c4c3)n2)cc1]

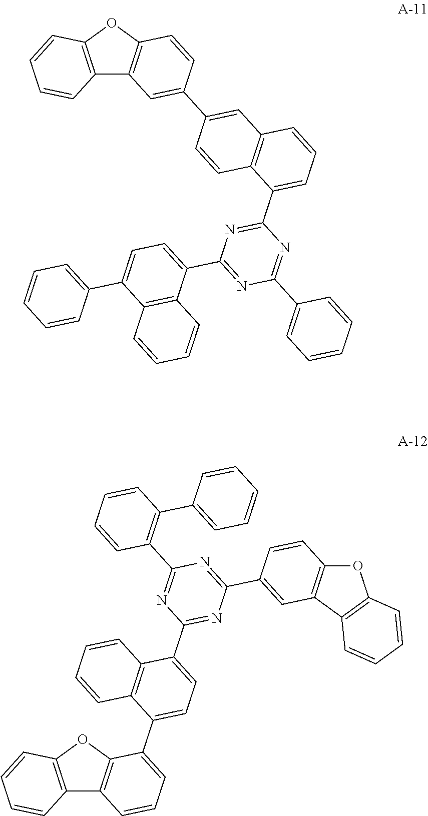 [SMILES: c1ccc(-c2nc(-c3cccc4cc(-c5ccc6oc7ccccc7c6c5)ccc34)nc(-c3ccc(-c4ccccc4)c4ccccc34)n2)cc1 ; c1ccc(-c2ccccc2-c2nc(-c3ccc4oc5ccccc5c4c3)nc(-c3ccc(-c4cccc5c4oc4ccccc45)c4ccccc34)n2)cc1]

### Comparative Examples 61 to 156

An organic light emitting device was manufactured in the same manner as in Example 1, except that in the organic light emitting device of Example 1, the compound represented by Chemical Formula 1 shown in Table 3 below was used instead of Compound 1-1 as the first host, and the following comparative compounds B-1 to B-12 were used instead of Compound 2-1 as the second host, wherein these two host compounds were co-deposited and used in a weight ratio of 1:1. The specific structures of the Compounds B-1 to B-12 are as follows.

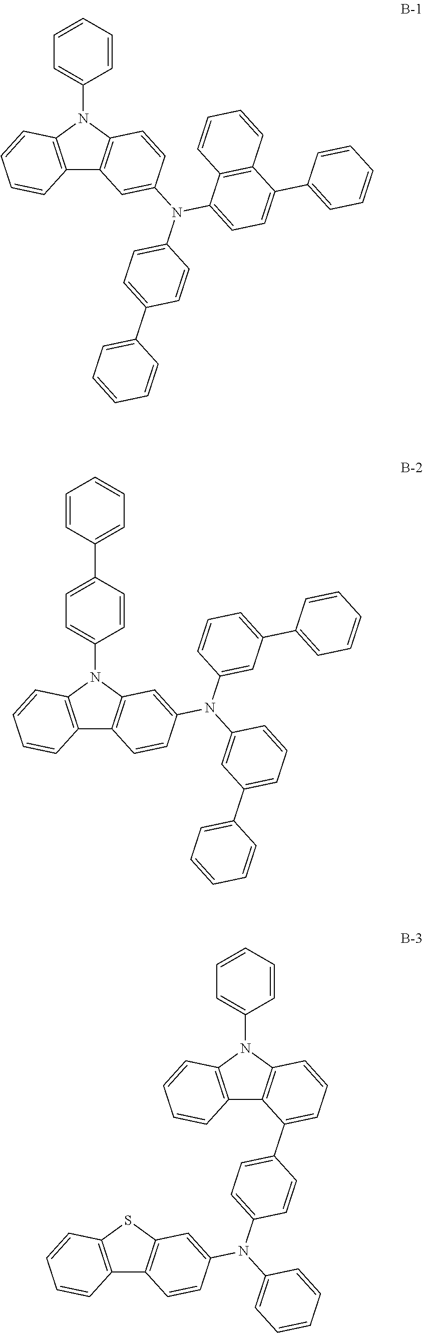 [SMILES: c1ccc(-c2ccc(N(c3ccc4c(c3)c3ccccc3n4-c3ccccc3)c3ccc(-c4ccccc4)c4ccccc34)cc2)cc1 ; c1ccc(-c2ccc(-n3c4ccccc4c4ccc(N(c5cccc(-c6ccccc6)c5)c5cccc(-c6ccccc6)c5)cc43)cc2)cc1 ; c1ccc(N(c2ccc(-c3cccc4c3c3ccccc3n4-c3ccccc3)cc2)c2ccc3c(c2)sc2ccccc23)cc1]

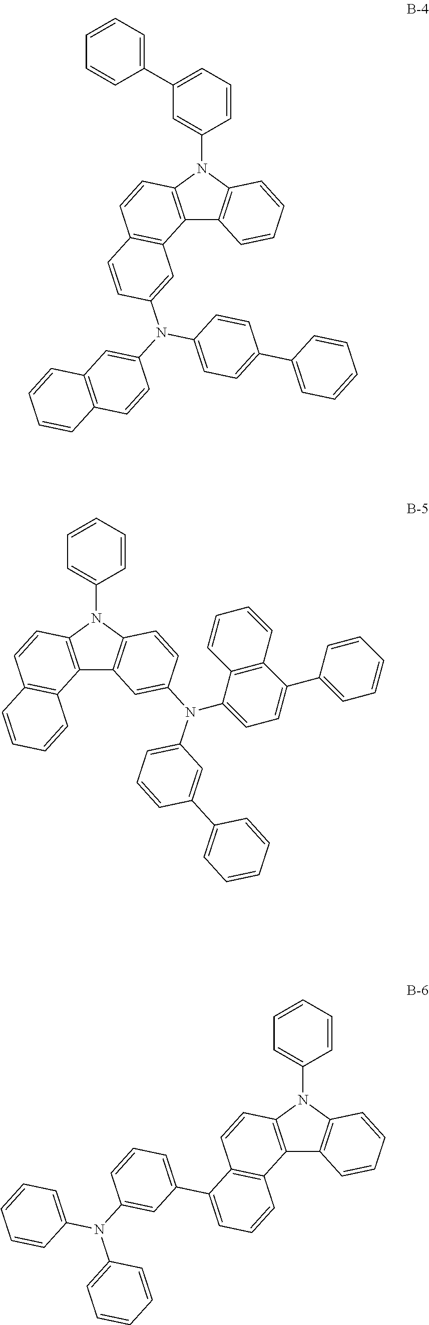 [SMILES: c1ccc(-c2ccc(N(c3ccc4ccccc4c3)c3ccc4ccc5c(c4c3)c3ccccc3n5-c3cccc(-c4ccccc4)c3)cc2)cc1 ; c1ccc(-c2cccc(N(c3ccc4c(c3)c3c5ccccc5ccc3n4-c3ccccc3)c3ccc(-c4ccccc4)c4ccccc34)c2)cc1 ; c1ccc(N(c2ccccc2)c2cccc(-c3cccc4c3ccc3c4c4ccccc4n3-c3ccccc3)c2)cc1]

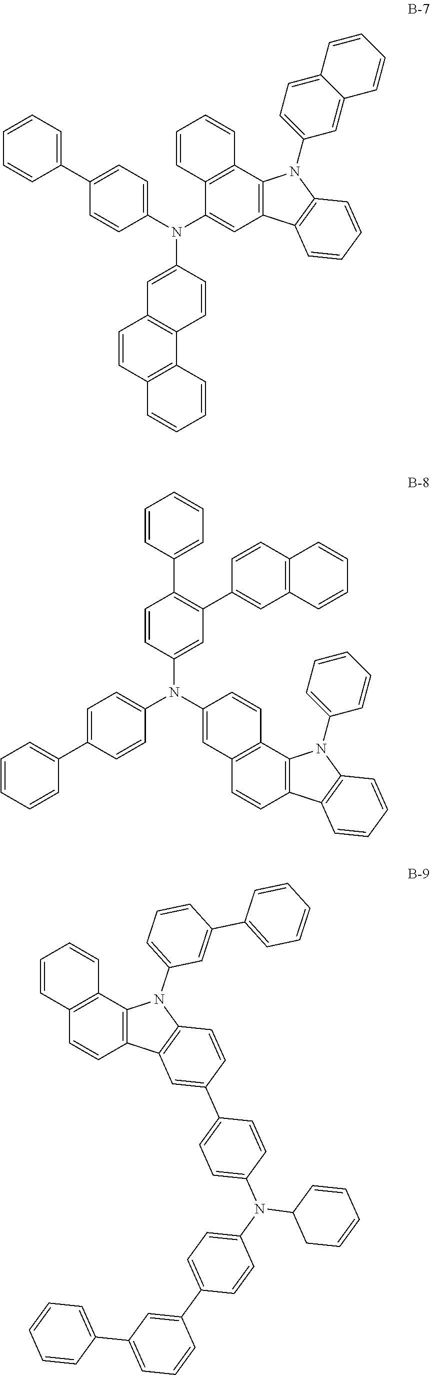 [SMILES: c1ccc(-c2ccc(N(c3ccc4c(ccc5ccccc54)c3)c3cc4c5ccccc5n(-c5ccc6ccccc6c5)c4c4ccccc34)cc2)cc1 ; c1ccc(-c2ccc(N(c3ccc(-c4ccccc4)c(-c4ccc5ccccc5c4)c3)c3ccc4c(ccc5c6ccccc6n(-c6ccccc6)c45)c3)cc2)cc1 ; C1=CCC(N(c2ccc(-c3cccc(-c4ccccc4)c3)cc2)c2ccc(-c3ccc4c(c3)c3ccc5ccccc5c3n4-c3cccc(-c4ccccc4)c3)cc2)C=C1]

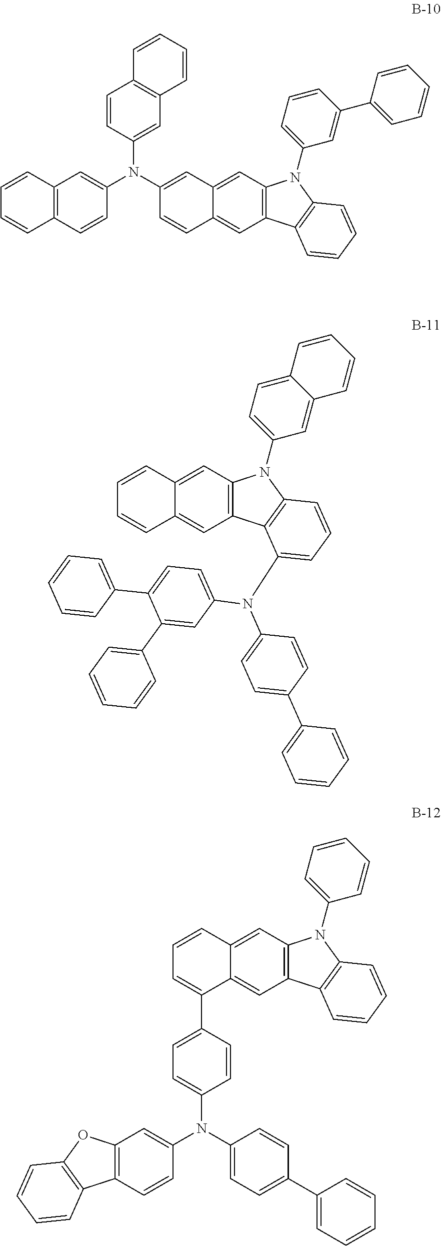 [SMILES: c1ccc(-c2cccc(-n3c4ccccc4c4cc5ccc(N(c6ccc7ccccc7c6)c6ccc7ccccc7c6)cc5cc43)c2)cc1 ; c1ccc(-c2ccc(N(c3ccc(-c4ccccc4)c(-c4ccccc4)c3)c3cccc4c3c3cc5ccccc5cc3n4-c3ccc4ccccc4c3)cc2)cc1 ; c1ccc(-c2ccc(N(c3ccc(-c4cccc5cc6c(cc45)c4ccccc4n6-c4ccccc4)cc3)c3ccc4c(c3)oc3ccccc34)cc2)cc1]

### Experimental Example

The voltage and efficiency were measured (based on 15 mA/cm²) by applying a current to the organic light emitting devices manufactured in Examples 1 to 340 and Comparative Examples 1 to 156, and the results are shown in Tables 1 to 3 below. The lifetime (T95) was measured based on 7000 nit, and T95 means the time required for the luminance to be reduced to 95% of the initial luminance.

TABLE 1

| | The | The | | | Lifetime | Lumi- |
|---|---|---|---|---|---|---|
| | First | Second | Voltage | Efficiency | T95 | nescent |
| Division | Host | Host | (V) | (cd/A) | (hr) | Color |
| Example | Compound | Compound | 3.74 | 20.22 | 207 | Red |
| 1 | 1-1 | 2-1 | | | | |
| Example | | Compound | 3.76 | 20.31 | 207 | Red |
| 2 | | 2-27 | | | | |
| Example | | Compound | 3.69 | 20.34 | 193 | Red |
| 3 | | 2-53 | | | | |
| Example | | Compound | 3.76 | 20.88 | 200 | Red |
| 4 | | 2-79 | | | | |
| Example | | Compound | 3.74 | 20.99 | 207 | Red |
| 5 | | 2-105 | | | | |
| Example | Compound | Compound | 3.71 | 21.08 | 190 | Red |
| 6 | 1-2 | 2-2 | | | | |
| Example | | Compound | 3.71 | 21.03 | 201 | Red |
| 7 | | 2-28 | | | | |
| Example | | Compound | 3.71 | 21.12 | 192 | Red |
| 8 | | 2-54 | | | | |
| Example | | Compound | 3.68 | 20.06 | 205 | Red |
| 9 | | 2-80 | | | | |
| Example | | Compound | 3.72 | 21.32 | 206 | Red |
| 10 | | 2-106 | | | | |
| Example | Compound | Compound | 3.45 | 22.88 | 237 | Red |
| 11 | 1-3 | 2-3 | | | | |
| Example | | Compound | 3.45 | 22.73 | 241 | Red |
| 12 | | 2-29 | | | | |
| Example | | Compound | 3.49 | 22.73 | 225 | Red |
| 13 | | 2-55 | | | | |
| Example | | Compound | 3.48 | 22.83 | 231 | Red |
| 14 | | 2-81 | | | | |
| Example | | Compound | 3.51 | 22.89 | 233 | Red |
| 15 | | 2-107 | | | | |
| Example | Compound | Compound | 3.47 | 22.82 | 223 | Red |
| 16 | 1-4 | 2-4 | | | | |
| Example | | Compound | 3.53 | 22.83 | 251 | Red |
| 17 | | 2-30 | | | | |
| Example | | Compound | 3.53 | 22.56 | 240 | Red |
| 18 | | 2-56 | | | | |
| Example | | Compound | 3.54 | 22.74 | 237 | Red |
| 19 | | 2-82 | | | | |
| Example | | Compound | 3.52 | 22.90 | 250 | Red |
| 20 | | 2-108 | | | | |
| Example | Compound | Compound | 3.60 | 22.88 | 237 | Red |
| 21 | 1-5 | 2-5 | | | | |
| Example | | Compound | 3.53 | 11.92 | 241 | Red |
| 22 | | 2-31 | | | | |
| Example | | Compound | 3.58 | 17.20 | 225 | Red |
| 23 | | 2-57 | | | | |
| Example | | Compound | 3.57 | 12.19 | 231 | Red |
| 24 | | 2-83 | | | | |
| Example | | Compound | 3.56 | 22.69 | 233 | Red |
| 25 | | 2-109 | | | | |
| Example | Compound | Compound | 3.53 | 18.83 | 223 | Red |
| 26 | 1-6 | 2-6 | | | | |
| Example | | Compound | 3.57 | 19.61 | 251 | Red |
| 27 | | 2-32 | | | | |
| Example | | Compound | 3.53 | 14.53 | 240 | Red |
| 28 | | 2-58 | | | | |
| Example | | Compound | 3.59 | 21.64 | 237 | Red |
| 29 | | 2-84 | | | | |
| Example | | Compound | 3.58 | 21.76 | 250 | Red |
| 30 | | 2-110 | | | | |
| Example | Compound | Compound | 3.53 | 22.69 | 270 | Red |
| 31 | 1-7 | 2-7 | | | | |
| Example | | Compound | 3.46 | 22.88 | 256 | Red |
| 32 | | 2-33 | | | | |
| Example | | Compound | 3.50 | 22.70 | 255 | Red |
| 33 | | 2-59 | | | | |
| Example | | Compound | 3.47 | 22.69 | 247 | Red |
| 34 | | 2-85 | | | | |
| Example | | Compound | 3.48 | 22.63 | 261 | Red |
| 35 | | 2-111 | | | | |
| Example | Compound | Compound | 3.54 | 22.78 | 258 | Red |
| 36 | 1-8 | 2-8 | | | | |
| Example | | Compound | 3.49 | 22.64 | 261 | Red |
| 37 | | 2-34 | | | | |
| Example | | Compound | 3.48 | 22.74 | 255 | Red |
| 38 | | 2-60 | | | | |
| Example | | Compound | 3.47 | 22.66 | 251 | Red |
| 39 | | 2-86 | | | | |
| Example | | Compound | 3.49 | 22.52 | 247 | Red |
| 40 | | 2-112 | | | | |
| Example | Compound | Compound | 3.53 | 23.45 | 270 | Red |
| 41 | 1-9 | 2-9 | | | | |
| Example | | Compound | 3.46 | 23.03 | 256 | Red |
| 42 | | 2-35 | | | | |
| Example | | Compound | 3.50 | 23.14 | 255 | Red |
| 43 | | 2-61 | | | | |
| Example | | Compound | 3.47 | 23.36 | 247 | Red |
| 44 | | 2-87 | | | | |
| Example | | Compound | 3.48 | 23.69 | 261 | Red |
| 45 | | 2-113 | | | | |
| Example | Compound | Compound | 3.49 | 23.99 | 308 | Red |
| 46 | 1-10 | 2-10 | | | | |
| Example | | Compound | 3.45 | 23.63 | 300 | Red |
| 47 | | 2-36 | | | | |
| Example | | Compound | 3.46 | 22.80 | 291 | Red |
| 48 | | 2-62 | | | | |
| Example | | Compound | 3.46 | 23.29 | 306 | Red |
| 49 | | 2-88 | | | | |
| Example | | Compound | 3.46 | 23.47 | 300 | Red |
| 50 | | 2-114 | | | | |
| Example | Compound | Compound | 3.45 | 23.43 | 284 | Red |
| 51 | 1-11 | 2-11 | | | | |
| Example | | Compound | 3.47 | 23.51 | 291 | Red |
| 52 | | 2-37 | | | | |
| Example | | Compound | 3.51 | 23.66 | 284 | Red |
| 53 | | 2-63 | | | | |
| Example | | Compound | 3.47 | 23.07 | 292 | Red |
| 54 | | 2-89 | | | | |
| Example | | Compound | 3.53 | 23.15 | 288 | Red |
| 55 | | 2-115 | | | | |
| Example | Compound | Compound | 3.54 | 22.78 | 234 | Red |
| 56 | 1-12 | 2-12 | | | | |
| Example | | Compound | 3.49 | 22.64 | 242 | Red |
| 57 | | 2-38 | | | | |
| Example | | Compound | 3.48 | 22.74 | 248 | Red |
| 58 | | 2-64 | | | | |
| Example | | Compound | 3.47 | 22.66 | 229 | Red |
| 59 | | 2-90 | | | | |
| Example | | Compound | 3.49 | 22.52 | 224 | Red |
| 60 | | 2-116 | | | | |
| Example | Compound | Compound | 3.61 | 22.69 | 225 | Red |
| 61 | 1-13 | 2-13 | | | | |
| Example | | Compound | 3.61 | 17.18 | 230 | Red |
| 62 | | 2-39 | | | | |
| Example | | Compound | 3.54 | 17.66 | 248 | Red |
| 63 | | 2-65 | | | | |
| Example | | Compound | 3.58 | 17.46 | 223 | Red |
| 64 | | 2-91 | | | | |
| Example | | Compound | 3.53 | 20.20 | 248 | Red |
| 65 | | 2-117 | | | | |
| Example | Compound | Compound | 3.53 | 22.06 | 234 | Red |
| 66 | 1-14 | 2-14 | | | | |
| Example | | Compound | 3.61 | 20.95 | 242 | Red |
| 67 | | 2-40 | | | | |
| Example | | Compound | 3.56 | 22.11 | 248 | Red |
| 68 | | 2-66 | | | | |
| Example | | Compound | 3.55 | 14.61 | 229 | Red |
| 69 | | 2-92 | | | | |
| Example | | Compound | 3.52 | 16.10 | 224 | Red |
| 70 | | 2-118 | | | | |
| Example | Compound | Compound | 3.71 | 20.37 | 192 | Red |
| 71 | 1-15 | 2-15 | | | | |
| Example | | Compound | 3.71 | 20.68 | 204 | Red |
| 72 | | 2-41 | | | | |
| Example | | Compound | 3.70 | 20.61 | 197 | Red |
| 73 | | 2-67 | | | | |
| Example | | Compound | 3.73 | 21.08 | 195 | Red |
| 74 | | 2-93 | | | | |
| Example | | Compound | 3.76 | 21.44 | 207 | Red |
| 75 | | 2-119 | | | | |
| Example | Compound | Compound | 3.64 | 14.88 | 227 | Red |
| 76 | 1-16 | 2-16 | | | | |
| Example | | Compound | 3.59 | 18.01 | 218 | Red |
| 77 | | 2-42 | | | | |
| Example | | Compound | 3.60 | 12.80 | 220 | Red |
| 78 | | 2-68 | | | | |
| Example | | Compound | 3.63 | 22.54 | 227 | Red |
| 79 | | 2-94 | | | | |
| Example | | Compound | 3.65 | 18.15 | 227 | Red |
| 80 | | 2-120 | | | | |
| Example | Compound | Compound | 3.62 | 20.02 | 225 | Red |
| 81 | 1-17 | 2-17 | | | | |
| Example | | Compound | 3.67 | 12.71 | 224 | Red |
| 82 | | 2-43 | | | | |
| Example | | Compound | 3.66 | 18.04 | 226 | Red |
| 83 | | 2-69 | | | | |
| Example | | Compound | 3.61 | 22.28 | 221 | Red |
| 84 | | 2-95 | | | | |
| Example | | Compound | 3.65 | 15.15 | 219 | Red |
| 85 | | 2-121 | | | | |
| Example | Compound | Compound | 3.54 | 22.56 | 244 | Red |
| 86 | 1-18 | 2-18 | | | | |
| Example | | Compound | 3.46 | 22.79 | 235 | Red |
| 87 | | 2-44 | | | | |
| Example | | Compound | 3.45 | 22.66 | 241 | Red |
| 88 | | 2-70 | | | | |
| Example | | Compound | 3.54 | 22.52 | 250 | Red |
| 89 | | 2-96 | | | | |
| Example | | Compound | 3.54 | 22.51 | 237 | Red |
| 90 | | 2-122 | | | | |
| Example | Compound | Compound | 3.50 | 22.65 | 235 | Red |
| 91 | 1-19 | 2-19 | | | | |
| Example | | Compound | 3.53 | 22.85 | 234 | Red |
| 92 | | 2-45 | | | | |
| Example | | Compound | 3.53 | 22.89 | 237 | Red |
| 93 | | 2-71 | | | | |
| Example | | Compound | 3.48 | 22.71 | 242 | Red |
| 94 | | 2-97 | | | | |
| Example | | Compound | 3.53 | 22.67 | 247 | Red |
| 95 | | 2-123 | | | | |
| Example | Compound | Compound | 3.58 | 22.56 | 244 | Red |
| 96 | 1-20 | 2-20 | | | | |
| Example | | Compound | 3.56 | 18.01 | 235 | Red |
| 97 | | 2-46 | | | | |
| Example | | Compound | 3.54 | 12.80 | 241 | Red |
| 98 | | 2-72 | | | | |
| Example | | Compound | 3.53 | 22.54 | 250 | Red |
| 99 | | 2-98 | | | | |
| Example | | Compound | 3.52 | 18.15 | 237 | Red |
| 100 | | 2-124 | | | | |
| Example | Compound | Compound | 3.60 | 20.02 | 235 | Red |
| 101 | 1-21 | 2-21 | | | | |
| Example | | Compound | 3.57 | 12.71 | 234 | Red |
| 102 | | 2-47 | | | | |
| Example | | Compound | 3.56 | 18.04 | 237 | Red |
| 103 | | 2-73 | | | | |
| Example | | Compound | 3.54 | 22.28 | 242 | Red |
| 104 | | 2-99 | | | | |
| Example | | Compound | 3.58 | 15.15 | 247 | Red |
| 105 | | 2-125 | | | | |
| Example | Compound | Compound | 3.49 | 22.61 | 237 | Red |
| 106 | 1-22 | 2-22 | | | | |
| Example | | Compound | 3.51 | 22.68 | 244 | Red |
| 107 | | 2-48 | | | | |
| Example | | Compound | 3.51 | 22.81 | 226 | Red |
| 108 | | 2-74 | | | | |
| Example | | Compound | 3.47 | 22.63 | 224 | Red |
| 109 | | 2-100 | | | | |
| Example | | Compound | 3.46 | 22.52 | 223 | Red |
| 110 | | 2-126 | | | | |
| Example | Compound | Compound | 3.51 | 22.55 | 246 | Red |
| 111 | 1-23 | 2-23 | | | | |
| Example | | Compound | 3.46 | 22.78 | 230 | Red |
| 112 | | 2-49 | | | | |
| Example | | Compound | 3.53 | 22.69 | 251 | Red |
| 113 | | 2-75 | | | | |
| Example | | Compound | 3.51 | 22.78 | 248 | Red |
| 114 | | 2-101 | | | | |
| Example | | Compound | 3.46 | 22.68 | 243 | Red |
| 115 | | 2-127 | | | | |
| Example | Compound | Compound | 3.49 | 23.86 | 272 | Red |
| 116 | 1-24 | 2-24 | | | | |
| Example | | Compound | 3.51 | 23.39 | 247 | Red |
| 117 | | 2-50 | | | | |
| Example | | Compound | 3.51 | 22.90 | 267 | Red |
| 118 | | 2-76 | | | | |
| Example | | Compound | 3.47 | 23.02 | 267 | Red |
| 119 | | 2-102 | | | | |
| Example | | Compound | 3.46 | 23.02 | 252 | Red |
| 120 | | 2-128 | | | | |
| Example | Compound | Compound | 3.54 | 22.81 | 312 | Red |
| 121 | 1-25 | 2-25 | | | | |
| Example | | Compound | 3.46 | 23.36 | 290 | Red |
| 122 | | 2-51 | | | | |
| Example | | Compound | 3.45 | 23.36 | 313 | Red |
| 123 | | 2-77 | | | | |
| Example | | Compound | 3.48 | 23.36 | 308 | Red |
| 124 | | 2-103 | | | | |
| Example | | Compound | 3.52 | 23.63 | 318 | Red |
| 125 | | 2-129 | | | | |
| Example | Compound | Compound | 3.50 | 23.38 | 315 | Red |
| 126 | 1-26 | 2-26 | | | | |
| Example | | Compound | 3.54 | 23.19 | 284 | Red |
| 127 | | 2-52 | | | | |
| Example | | Compound | 3.54 | 22.88 | 297 | Red |
| 128 | | 2-78 | | | | |
| Example | | Compound | 3.49 | 23.44 | 320 | Red |
| 129 | | 2-104 | | | | |
| Example | | Compound | 3.52 | 22.85 | 311 | Red |
| 130 | | 2-130 | | | | |
| Example | Compound | Compound | 3.54 | 22.89 | 284 | Red |
| 131 | 1-27 | 2-21 | | | | |
| Example | | Compound | 3.53 | 22.51 | 286 | Red |
| 132 | | 2-49 | | | | |
| Example | | Compound | 3.51 | 22.86 | 303 | Red |
| 133 | | 2-73 | | | | |
| Example | | Compound | 3.48 | 22.86 | 284 | Red |
| 134 | | 2-102 | | | | |
| Example | | Compound | 3.54 | 22.75 | 317 | Red |
| 135 | | 2-130 | | | | |
| Example | Compound | Compound | 3.63 | 20.16 | 216 | Red |
| 136 | 1-28 | 2-1 | | | | |
| Example | | Compound | 3.65 | 15.13 | 213 | Red |
| 137 | | 2-27 | | | | |
| Example | | Compound | 3.59 | 19.04 | 214 | Red |
| 138 | | 2-53 | | | | |
| Example | | Compound | 3.64 | 11.67 | 219 | Red |
| 139 | | 2-79 | | | | |
| Example | | Compound | 3.61 | 20.90 | 214 | Red |
| 140 | | 2-105 | | | | |
| Example | Compound | Compound | 3.50 | 22.65 | 240 | Red |
| 141 | 1-29 | 2-2 | | | | |
| Example | | Compound | 3.54 | 22.60 | 250 | Red |
| 142 | | 2-28 | | | | |
| Example | | Compound | 3.49 | 22.53 | 229 | Red |
| 143 | | 2-54 | | | | |
| Example | | Compound | 3.48 | 22.59 | 238 | Red |
| 144 | | 2-80 | | | | |
| Example | | Compound | 3.50 | 22.88 | 233 | Red |
| 145 | | 2-106 | | | | |
| Example | Compound | Compound | 3.53 | 22.54 | 230 | Red |
| 146 | 1-30 | 2-3 | | | | |
| Example | | Compound | 3.51 | 22.64 | 231 | Red |
| 147 | | 2-29 | | | | |
| Example | | Compound | 3.46 | 22.56 | 228 | Red |
| 148 | | 2-55 | | | | |
| Example | | Compound | 3.49 | 22.89 | 227 | Red |
| 149 | | 2-81 | | | | |
| Example | | Compound | 3.50 | 22.72 | 243 | Red |
| 150 | | 2-107 | | | | |
| Example | Compound | Compound | 3.61 | 22.65 | 240 | Red |
| 151 | 1-31 | 2-4 | | | | |
| Example | | Compound | 3.60 | 22.17 | 250 | Red |
| 152 | | 2-30 | | | | |
| Example | | Compound | 3.54 | 15.76 | 229 | Red |
| 153 | | 2-56 | | | | |
| Example | | Compound | 3.55 | 16.14 | 238 | Red |
| 154 | | 2-82 | | | | |
| Example | | Compound | 3.58 | 14.83 | 233 | Red |
| 155 | | 2-108 | | | | |
| Example | Compound | Compound | 3.52 | 20.16 | 230 | Red |
| 156 | 1-32 | 2-5 | | | | |
| Example | | Compound | 3.56 | 15.13 | 231 | Red |
| 157 | | 2-31 | | | | |
| Example | | Compound | 3.58 | 19.04 | 228 | Red |
| 158 | | 2-57 | | | | |
| Example | | Compound | 3.58 | 11.67 | 227 | Red |
| 159 | | 2-83 | | | | |
| Example | | Compound | 3.56 | 20.90 | 243 | Red |
| 160 | | 2-109 | | | | |
| Example | Compound | Compound | 3.57 | 22.76 | 229 | Red |
| 161 | 1-33 | 2-6 | | | | |
| Example | | Compound | 3.56 | 20.69 | 242 | Red |
| 162 | | 2-32 | | | | |
| Example | | Compound | 3.58 | 14.71 | 232 | Red |
| 163 | | 2-58 | | | | |
| Example | | Compound | 3.52 | 15.56 | 232 | Red |
| 164 | | 2-84 | | | | |
| Example | | Compound | 3.52 | 19.56 | 245 | Red |
| 165 | | 2-110 | | | | |
| Example | Compound | Compound | 3.52 | 22.83 | 318 | Red |
| 166 | 1-34 | 2-7 | | | | |
| Example | | Compound | 3.46 | 23.64 | 287 | Red |
| 167 | | 2-33 | | | | |
| Example | | Compound | 3.52 | 23.12 | 297 | Red |
| 168 | | 2-59 | | | | |
| Example | | Compound | 3.45 | 23.67 | 281 | Red |
| 169 | | 2-85 | | | | |
| Example | | Compound | 3.49 | 23.23 | 298 | Red |
| 170 | | 2-111 | | | | |
| Example | Compound | Compound | 3.49 | 22.94 | 295 | Red |
| 171 | 1-35 | 2-8 | | | | |
| Example | | Compound | 3.45 | 23.02 | 289 | Red |
| 172 | | 2-34 | | | | |
| Example | | Compound | 3.51 | 23.04 | 311 | Red |
| 173 | | 2-60 | | | | |
| Example | | Compound | 3.53 | 22.80 | 304 | Red |
| 174 | | 2-86 | | | | |
| Example | | Compound | 3.53 | 23.24 | 288 | Red |
| 175 | | 2-112 | | | | |
| Example | Compound | Compound | 3.47 | 22.85 | 293 | Red |
| 176 | 1-36 | 2-9 | | | | |
| Example | | Compound | 3.46 | 22.78 | 309 | Red |
| 177 | | 2-35 | | | | |
| Example | | Compound | 3.52 | 22.74 | 315 | Red |
| 178 | | 2-61 | | | | |
| Example | | Compound | 3.49 | 22.78 | 318 | Red |
| 179 | | 2-87 | | | | |
| Example | | Compound | 3.49 | 22.77 | 309 | Red |
| 180 | | 2-113 | | | | |
| Example | Compound | Compound | 3.53 | 22.58 | 233 | Red |
| 181 | 1-37 | 2-10 | | | | |
| Example | | Compound | 3.54 | 22.51 | 251 | Red |
| 182 | | 2-36 | | | | |
| Example | | Compound | 3.51 | 22.60 | 236 | Red |
| 183 | | 2-62 | | | | |
| Example | | Compound | 3.45 | 22.87 | 246 | Red |
| 184 | | 2-88 | | | | |
| Example | | Compound | 3.45 | 22.66 | 228 | Red |
| 185 | | 2-114 | | | | |
| Example | Compound | Compound | 3.49 | 22.64 | 289 | Red |
| 186 | 1-38 | 2-11 | | | | |
| Example | | Compound | 3.49 | 22.63 | 299 | Red |
| 187 | | 2-37 | | | | |
| Example | | Compound | 3.53 | 22.52 | 305 | Red |
| 188 | | 2-63 | | | | |
| Example | | Compound | 3.47 | 22.80 | 291 | Red |
| 189 | | 2-89 | | | | |
| Example | | Compound | 3.50 | 22.74 | 285 | Red |
| 190 | | 2-115 | | | | |
| Example | Compound | Compound | 3.55 | 22.58 | 233 | Red |
| 191 | 1-39 | 2-12 | | | | |
| Example | | Compound | 3.56 | 21.83 | 251 | Red |
| 192 | | 2-38 | | | | |
| Example | | Compound | 3.61 | 21.20 | 236 | Red |
| 193 | | 2-64 | | | | |
| Example | | Compound | 3.58 | 16.40 | 246 | Red |
| 194 | | 2-90 | | | | |
| Example | | Compound | 3.55 | 14.51 | 228 | Red |
| 195 | | 2-116 | | | | |
| Example | Compound | Compound | 3.54 | 15.42 | 240 | Red |
| 196 | 1-40 | 2-13 | | | | |
| Example | | Compound | 3.55 | 19.92 | 244 | Red |
| 197 | | 2-39 | | | | |
| Example | | Compound | 3.57 | 16.08 | 231 | Red |
| 198 | | 2-65 | | | | |
| Example | | Compound | 3.60 | 20.30 | 223 | Red |
| 199 | | 2-91 | | | | |
| Example | | Compound | 3.54 | 21.28 | 248 | Red |
| 200 | | 2-117 | | | | |
| Example | Compound | Compound | 3.66 | 20.31 | 226 | Red |
| 201 | 1-41 | 2-14 | | | | |
| Example | | Compound | 3.64 | 21.83 | 211 | Red |
| 202 | | 2-40 | | | | |
| Example | | Compound | 3.61 | 21.20 | 218 | Red |
| 203 | | 2-66 | | | | |
| Example | | Compound | 3.67 | 16.40 | 221 | Red |
| 204 | | 2-92 | | | | |
| Example | | Compound | 3.67 | 14.51 | 218 | Red |
| 205 | | 2-118 | | | | |
| Example | Compound | Compound | 3.64 | 15.42 | 228 | Red |
| 206 | 1-42 | 2-15 | | | | |
| Example | | Compound | 3.64 | 19.92 | 219 | Red |
| 207 | | 2-41 | | | | |
| Example | | Compound | 3.65 | 16.08 | 221 | Red |
| 208 | | 2-67 | | | | |
| Example | | Compound | 3.59 | 20.30 | 224 | Red |
| 209 | | 2-93 | | | | |
| Example | | Compound | 3.65 | 21.28 | 223 | Red |
| 210 | | 2-119 | | | | |
| Example | Compound | Compound | 3.52 | 22.78 | 286 | Red |
| 211 | 1-43 | 2-16 | | | | |
| Example | | Compound | 3.54 | 22.61 | 312 | Red |
| 212 | | 2-42 | | | | |
| Example | | Compound | 3.51 | 22.54 | 284 | Red |
| 213 | | 2-68 | | | | |
| Example | | Compound | 3.52 | 22.67 | 317 | Red |
| 214 | | 2-94 | | | | |
| Example | | Compound | 3.48 | 22.89 | 319 | Red |
| 215 | | 2-120 | | | | |
| Example | Compound | Compound | 3.66 | 20.31 | 226 | Red |
| 216 | 1-44 | 2-17 | | | | |
| Example | | Compound | 3.64 | 21.83 | 211 | Red |
| 217 | | 2-43 | | | | |
| Example | | Compound | 3.61 | 21.20 | 218 | Red |
| 218 | | 2-69 | | | | |
| Example | | Compound | 3.67 | 16.40 | 221 | Red |
| 219 | | 2-95 | | | | |
| Example | | Compound | 3.67 | 14.51 | 218 | Red |
| 220 | | 2-121 | | | | |
| Example | Compound | Compound | 3.64 | 15.42 | 228 | Red |
| 221 | 1-45 | 2-18 | | | | |
| Example | | Compound | 3.64 | 19.92 | 219 | Red |
| 222 | | 2-44 | | | | |
| Example | | Compound | 3.65 | 16.08 | 221 | Red |
| 223 | | 2-70 | | | | |
| Example | | Compound | 3.59 | 20.30 | 224 | Red |
| 224 | | 2-96 | | | | |
| Example | | Compound | 3.65 | 21.28 | 223 | Red |
| 225 | | 2-122 | | | | |
| Example | Compound | Compound | 3.70 | 20.13 | 197 | Red |
| 226 | 1-46 | 2-19 | | | | |
| Example | | Compound | 3.76 | 21.45 | 198 | Red |
| 227 | | 2-45 | | | | |
| Example | | Compound | 3.73 | 20.08 | 197 | Red |
| 228 | | 2-71 | | | | |
| Example | | Compound | 3.72 | 21.05 | 207 | Red |
| 229 | | 2-97 | | | | |
| Example | | Compound | 3.71 | 20.26 | 192 | Red |
| 230 | | 2-123 | | | | |
| Example | Compound | Compound | 3.72 | 21.19 | 197 | Red |
| 231 | 1-47 | 2-20 | | | | |
| Example | | Compound | 3.66 | 21.04 | 207 | Red |
| 232 | | 2-46 | | | | |
| Example | | Compound | 3.69 | 21.10 | 208 | Red |
| 233 | | 2-72 | | | | |
| Example | | Compound | 3.68 | 21.03 | 197 | Red |
| 234 | | 2-98 | | | | |
| Example | | Compound | 3.74 | 21.21 | 201 | Red |
| 235 | | 2-124 | | | | |
| Example | Compound | Compound | 3.72 | 21.24 | 198 | Red |
| 236 | 1-48 | 2-1 | | | | |
| Example | | Compound | 3.74 | 20.24 | 202 | Red |
| 237 | | 2-17 | | | | |
| Example | | Compound | 3.72 | 21.41 | 192 | Red |
| 238 | | 2-43 | | | | |
| Example | | Compound | 3.74 | 20.29 | 201 | Red |
| 239 | | 2-89 | | | | |
| Example | | Compound | 3.77 | 21.50 | 208 | Red |
| 240 | | 2-105 | | | | |
| Example | Compound | Compound | 3.72 | 20.43 | 192 | Red |
| 241 | 1-49 | 2-2 | | | | |
| Example | | Compound | 3.72 | 20.03 | 204 | Red |
| 242 | | 2-28 | | | | |
| Example | | Compound | 3.66 | 20.48 | 201 | Red |
| 243 | | 2-44 | | | | |
| Example | | Compound | 3.71 | 20.14 | 206 | Red |
| 244 | | 2-70 | | | | |
| Example | | Compound | 3.78 | 20.56 | 198 | Red |
| 245 | | 2-106 | | | | |
| Example | Compound | Compound | 3.60 | 22.65 | 225 | Red |
| 246 | 1-50 | 2-3 | | | | |
| Example | | Compound | 3.56 | 19.31 | 238 | Red |
| 247 | | 2-29 | | | | |
| Example | | Compound | 3.58 | 19.06 | 237 | Red |
| 248 | | 2-65 | | | | |
| Example | | Compound | 3.52 | 19.20 | 230 | Red |
| 249 | | 2-81 | | | | |
| Example | | Compound | 3.57 | 13.34 | 237 | Red |
| 250 | | 2-107 | | | | |
| Example | Compound | Compound | 3.62 | 21.52 | 216 | Red |
| 251 | 1-51 | 2-4 | | | | |
| Example | | Compound | 3.62 | 14.88 | 226 | Red |
| 252 | | 2-30 | | | | |
| Example | | Compound | 3.61 | 21.55 | 219 | Red |
| 253 | | 2-56 | | | | |
| Example | | Compound | 3.67 | 13.81 | 212 | Red |
| 254 | | 2-82 | | | | |
| Example | | Compound | 3.63 | 15.26 | 217 | Red |
| 255 | | 2-108 | | | | |
| Example | Compound | Compound | 3.60 | 18.19 | 226 | Red |
| 256 | 1-52 | 2-5 | | | | |
| Example | | Compound | 3.63 | 21.79 | 219 | Red |
| 257 | | 2-31 | | | | |
| Example | | Compound | 3.63 | 11.73 | 219 | Red |
| 258 | | 2-57 | | | | |
| Example | | Compound | 3.65 | 13.94 | 223 | Red |
| 259 | | 2-83 | | | | |
| Example | | Compound | 3.66 | 14.70 | 220 | Red |
| 260 | | 2-109 | | | | |
| Example | Compound | Compound | 3.51 | 22.71 | 239 | Red |
| 261 | 1-53 | 2-6 | | | | |
| Example | | Compound | 3.53 | 22.87 | 233 | Red |
| 262 | | 2-32 | | | | |
| Example | | Compound | 3.54 | 22.76 | 237 | Red |
| 263 | | 2-58 | | | | |
| Example | | Compound | 3.52 | 22.57 | 250 | Red |
| 264 | | 2-84 | | | | |
| Example | | Compound | 3.52 | 22.55 | 250 | Red |
| 265 | | 2-110 | | | | |
| Example | Compound | Compound | 3.54 | 22.90 | 320 | Red |
| 266 | 1-54 | 2-7 | | | | |
| Example | | Compound | 3.49 | 23.79 | 285 | Red |
| 267 | | 2-33 | | | | |
| Example | | Compound | 3.46 | 23.66 | 312 | Red |
| 268 | | 2-59 | | | | |
| Example | | Compound | 3.54 | 23.28 | 310 | Red |
| 269 | | 2-85 | | | | |
| Example | | Compound | 3.46 | 23.38 | 290 | Red |
| 270 | | 2-111 | | | | |
| Example | Compound | Compound | 3.46 | 22.50 | 290 | Red |
| 271 | 1-55 | 2-8 | | | | |
| Example | | Compound | 3.49 | 22.69 | 316 | Red |
| 272 | | 2-34 | | | | |
| Example | | Compound | 3.51 | 22.64 | 316 | Red |
| 273 | | 2-60 | | | | |
| Example | | Compound | 3.53 | 22.51 | 288 | Red |
| 274 | | 2-86 | | | | |
| Example | | Compound | 3.45 | 22.55 | 281 | Red |
| 275 | | 2-112 | | | | |
| Example | Compound | Compound | 3.73 | 20.14 | 202 | Red |
| 276 | 1-56 | 2-9 | | | | |
| Example | | Compound | 3.68 | 20.74 | 192 | Red |
| 277 | | 2-35 | | | | |
| Example | | Compound | 3.74 | 20.67 | 190 | Red |
| 278 | | 2-61 | | | | |
| Example | | Compound | 3.76 | 20.86 | 193 | Red |
| 279 | | 2-87 | | | | |
| Example | | Compound | 3.70 | 20.89 | 206 | Red |
| 280 | | 2-113 | | | | |
| Example | Compound | Compound | 3.61 | 22.77 | 223 | Red |
| 281 | 1-57 | 2-10 | | | | |
| Example | | Compound | 3.53 | 21.44 | 251 | Red |
| 282 | | 2-36 | | | | |
| Example | | Compound | 3.61 | 22.34 | 249 | Red |
| 283 | | 2-62 | | | | |
| Example | | Compound | 3.53 | 18.11 | 251 | Red |
| 284 | | 2-88 | | | | |
| Example | | Compound | 3.59 | 20.00 | 245 | Red |
| 285 | | 2-114 | | | | |
| Example | Compound | Compound | 3.61 | 19.47 | 230 | Red |
| 286 | 1-58 | 2-11 | | | | |
| Example | | Compound | 3.58 | 13.07 | 229 | Red |
| 287 | | 2-37 | | | | |
| Example | | Compound | 3.58 | 12.18 | 234 | Red |
| 288 | | 2-63 | | | | |
| Example | | Compound | 3.59 | 15.54 | 243 | Red |
| 289 | | 2-89 | | | | |
| Example | | Compound | 3.57 | 12.56 | 246 | Red |
| 290 | | 2-115 | | | | |
| Example | Compound | Compound | 3.47 | 22.97 | 318 | Red |
| 291 | 1-59 | 2-12 | | | | |
| Example | | Compound | 3.50 | 23.06 | 280 | Red |
| 292 | | 2-38 | | | | |
| Example | | Compound | 3.45 | 23.76 | 285 | Red |
| 293 | | 2-64 | | | | |
| Example | | Compound | 3.49 | 23.19 | 292 | Red |
| 294 | | 2-90 | | | | |
| Example | | Compound | 3.54 | 22.94 | 290 | Red |
| 295 | | 2-116 | | | | |
| Example | Compound | Compound | 3.48 | 23.59 | 292 | Red |
| 296 | 1-60 | 2-13 | | | | |
| Example | | Compound | 3.49 | 23.12 | 295 | Red |
| 297 | | 2-39 | | | | |
| Example | | Compound | 3.54 | 23.16 | 304 | Red |
| 298 | | 2-65 | | | | |
| Example | | Compound | 3.51 | 23.63 | 313 | Red |
| 299 | | 2-91 | | | | |
| Example | | Compound | 3.47 | 23.84 | 301 | Red |
| 300 | | 2-117 | | | | |
| Example | Compound | Compound | 3.70 | 20.29 | 198 | Red |
| 301 | 1-61 | 2-14 | | | | |
| Example | | Compound | 3.74 | 20.22 | 198 | Red |
| 302 | | 2-40 | | | | |
| Example | | Compound | 3.76 | 20.36 | 200 | Red |
| 303 | | 2-66 | | | | |
| Example | | Compound | 3.66 | 21.28 | 199 | Red |
| 304 | | 2-92 | | | | |
| Example | | Compound | 3.72 | 21.33 | 199 | Red |
| 305 | | 2-118 | | | | |
| Example | Compound | Compound | 3.70 | 20.30 | 208 | Red |
| 306 | 1-62 | 2-15 | | | | |
| Example | | Compound | 3.76 | 20.96 | 194 | Red |
| 307 | | 2-41 | | | | |
| Example | | Compound | 3.67 | 21.33 | 208 | Red |
| 308 | | 2-77 | | | | |
| Example | | Compound | 3.68 | 21.11 | 202 | Red |
| 309 | | 2-83 | | | | |
| Example | | Compound | 3.65 | 20.31 | 194 | Red |
| 310 | | 2-119 | | | | |
| Example | Compound | Compound | 3.62 | 10.98 | 220 | Red |
| 311 | 1-63 | 2-16 | | | | |
| Example | | Compound | 3.59 | 18.08 | 228 | Red |
| 312 | | 2-42 | | | | |
| Example | | Compound | 3.64 | 14.42 | 218 | Red |
| 313 | | 2-68 | | | | |
| Example | | Compound | 3.60 | 17.27 | 224 | Red |
| 314 | | 2-94 | | | | |
| Example | | Compound | 3.61 | 13.41 | 222 | Red |
| 315 | | 2-120 | | | | |
| Example | Compound | Compound | 3.48 | 22.55 | 313 | Red |
| 316 | 1-64 | 2-17 | | | | |
| Example | | Compound | 3.49 | 22.90 | 289 | Red |
| 317 | | 2-43 | | | | |
| Example | | Compound | 3.46 | 22.73 | 286 | Red |
| 318 | | 2-79 | | | | |
| Example | | Compound | 3.54 | 22.61 | 309 | Red |
| 319 | | 2-95 | | | | |
| Example | | Compound | 3.48 | 22.88 | 288 | Red |
| 320 | | 2-121 | | | | |
| Example | Compound | Compound | 3.52 | 23.24 | 302 | Red |
| 321 | 1-65 | 2-18 | | | | |
| Example | | Compound | 3.54 | 23.39 | 317 | Red |
| 322 | | 2-44 | | | | |
| Example | | Compound | 3.52 | 23.05 | 289 | Red |
| 323 | | 2-70 | | | | |
| Example | | Compound | 3.45 | 23.69 | 299 | Red |
| 324 | | 2-96 | | | | |
| Example | | Compound | 3.48 | 23.76 | 283 | Red |
| 325 | | 2-122 | | | | |
| Example | Compound | Compound | 3.67 | 20.13 | 199 | Red |
| 326 | 1-66 | 2-19 | | | | |
| Example | | Compound | 3.67 | 21.21 | 196 | Red |
| 327 | | 2-45 | | | | |
| Example | | Compound | 3.73 | 20.57 | 203 | Red |
| 328 | | 2-71 | | | | |
| Example | | Compound | 3.70 | 20.98 | 204 | Red |
| 329 | | 2-97 | | | | |
| Example | | Compound | 3.69 | 20.58 | 196 | Red |
| 330 | | 2-123 | | | | |
| Example | Compound | Compound | 3.49 | 22.79 | 295 | Red |
| 331 | 1-67 | 2-20 | | | | |
| Example | | Compound | 3.50 | 22.80 | 305 | Red |
| 332 | | 2-66 | | | | |
| Example | | Compound | 3.48 | 22.77 | 286 | Red |
| 333 | | 2-82 | | | | |
| Example | | Compound | 3.52 | 22.86 | 320 | Red |
| 334 | | 2-103 | | | | |
| Example | | Compound | 3.46 | 22.82 | 280 | Red |
| 335 | | 2-127 | | | | |
| Example | Compound | Compound | 3.46 | 22.85 | 288 | Red |
| 336 | 1-68 | 2-21 | | | | |
| Example | | Compound | 3.48 | 23.02 | 305 | Red |
| 337 | | 2-57 | | | | |
| Example | | Compound | 3.50 | 23.70 | 306 | Red |
| 338 | | 2-83 | | | | |
| Example | | Compound | 3.53 | 23.88 | 307 | Red |
| 339 | | 2-123 | | | | |
| Example | | Compound | 3.48 | 22.97 | 291 | Red |
| 340 | | 2-130 | | | | |

TABLE 2

| | | | | | Lifetime | |
|---|---|---|---|---|---|---|
| | The First | The Second | Voltage | Efficiency | T95 | Luminescent |
| Division | Host | Host | (V) | (cd/A) | (hr) | Color |
| Comparative | Compound | Compound | 4.13 | 16.63 | 129 | Red |
| Example 1 | A-1 | 2-1 | | | | |
| Comparative | | Compound | 4.08 | 16.45 | 129 | Red |
| Example 2 | | 2-27 | | | | |
| Comparative | | Compound | 4.17 | 17.07 | 124 | Red |
| Example 3 | | 2-53 | | | | |
| Comparative | | Compound | 4.10 | 16.96 | 126 | Red |
| Example 4 | | 2-79 | | | | |
| Comparative | | Compound | 4.15 | 16.56 | 146 | Red |
| Example 5 | | 2-105 | | | | |
| Comparative | Compound | Compound | 3.93 | 17.63 | 159 | Red |
| Example 6 | A-2 | 2-2 | | | | |
| Comparative | | Compound | 3.95 | 17.53 | 147 | Red |
| Example 7 | | 2-28 | | | | |
| Comparative | | Compound | 3.90 | 16.99 | 160 | Red |
| Example 8 | | 2-54 | | | | |
| Comparative | | Compound | 3.92 | 17.44 | 147 | Red |
| Example 9 | | 2-80 | | | | |
| Comparative | | Compound | 3.91 | 17.23 | 163 | Red |
| Example 10 | | 2-106 | | | | |
| Comparative | Compound | Compound | 3.95 | 16.63 | 129 | Red |
| Example 11 | A-3 | 2-3 | | | | |
| Comparative | | Compound | 3.90 | 16.45 | 129 | Red |
| Example 12 | | 2-29 | | | | |
| Comparative | | Compound | 3.94 | 17.07 | 124 | Red |
| Example 13 | | 2-55 | | | | |
| Comparative | | Compound | 3.88 | 16.96 | 126 | Red |
| Example 14 | | 2-81 | | | | |
| Comparative | | Compound | 3.88 | 16.56 | 146 | Red |
| Example 15 | | 2-107 | | | | |
| Comparative | Compound | Compound | 3.91 | 17.66 | 165 | Red |
| Example 16 | A-4 | 2-5 | | | | |
| Comparative | | Compound | 3.95 | 17.40 | 146 | Red |
| Example 17 | | 2-31 | | | | |
| Comparative | | Compound | 3.92 | 18.00 | 169 | Red |
| Example 18 | | 2-57 | | | | |
| Comparative | | Compound | 3.92 | 17.83 | 162 | Red |
| Example 19 | | 2-83 | | | | |
| Comparative | | Compound | 3.94 | 17.68 | 147 | Red |
| Example 20 | | 2-109 | | | | |
| Comparative | Compound | Compound | 3.89 | 17.54 | 169 | Red |
| Example 21 | A-5 | 2-8 | | | | |
| Comparative | | Compound | 3.95 | 17.05 | 163 | Red |
| Example 22 | | 2-34 | | | | |
| Comparative | | Compound | 3.88 | 17.31 | 152 | Red |
| Example 23 | | 2-60 | | | | |
| Comparative | | Compound | 3.95 | 17.67 | 153 | Red |
| Example 24 | | 2-86 | | | | |
| Comparative | | Compound | 3.93 | 17.68 | 162 | Red |
| Example 25 | | 2-112 | | | | |
| Comparative | Compound | Compound | 3.99 | 17.66 | 165 | Red |
| Example 26 | A-6 | 2-10 | | | | |
| Comparative | | Compound | 3.95 | 17.40 | 146 | Red |
| Example 27 | | 2-36 | | | | |
| Comparative | | Compound | 3.89 | 18.00 | 169 | Red |
| Example 28 | | 2-62 | | | | |
| Comparative | | Compound | 3.91 | 17.83 | 162 | Red |
| Example 29 | | 2-88 | | | | |
| Comparative | | Compound | 3.93 | 17.68 | 147 | Red |
| Example 30 | | 2-114 | | | | |
| Comparative | Compound | Compound | 3.90 | 17.54 | 169 | Red |
| Example 31 | A-7 | 2-12 | | | | |
| Comparative | | Compound | 3.92 | 17.05 | 163 | Red |
| Example 32 | | 2-38 | | | | |
| Comparative | | Compound | 3.90 | 17.31 | 152 | Red |
| Example 33 | | 2-64 | | | | |
| Comparative | | Compound | 3.95 | 17.67 | 153 | Red |
| Example 34 | | 2-90 | | | | |
| Comparative | | Compound | 3.94 | 17.68 | 162 | Red |
| Example 35 | | 2-116 | | | | |
| Comparative | Compound | Compound | 3.90 | 17.21 | 158 | Red |
| Example 36 | A-8 | 2-14 | | | | |
| Comparative | | Compound | 3.93 | 17.86 | 156 | Red |
| Example 37 | | 2-40 | | | | |
| Comparative | | Compound | 3.91 | 17.88 | 167 | Red |
| Example 38 | | 2-66 | | | | |
| Comparative | | Compound | 3.88 | 17.67 | 146 | Red |
| Example 39 | | 2-92 | | | | |
| Comparative | | Compound | 3.92 | 17.98 | 154 | Red |
| Example 40 | | 2-118 | | | | |
| Comparative | Compound | Compound | 3.91 | 17.73 | 170 | Red |
| Example 41 | A-9 | 2-17 | | | | |
| Comparative | | Compound | 3.93 | 17.02 | 150 | Red |
| Example 42 | | 2-43 | | | | |
| Comparative | | Compound | 3.93 | 17.14 | 146 | Red |
| Example 43 | | 2-69 | | | | |
| Comparative | | Compound | 3.94 | 17.44 | 169 | Red |
| Example 44 | | 2-95 | | | | |
| Comparative | | Compound | 3.93 | 17.05 | 146 | Red |
| Example 45 | | 2-121 | | | | |
| Comparative | Compound | Compound | 4.14 | 15.33 | 120 | Red |
| Example 46 | A-10 | 2-22 | | | | |
| Comparative | | Compound | 4.06 | 14.58 | 98 | Red |
| Example 47 | | 2-48 | | | | |
| Comparative | | Compound | 4.07 | 15.50 | 113 | Red |
| Example 48 | | 2-74 | | | | |
| Comparative | | Compound | 4.11 | 15.35 | 107 | Red |
| Example 49 | | 2-100 | | | | |
| Comparative | | Compound | 4.12 | 16.04 | 108 | Red |
| Example 50 | | 2-126 | | | | |
| Comparative | Compound | Compound | 3.93 | 16.87 | 126 | Red |
| Example 51 | A-11 | 2-26 | | | | |
| Comparative | | Compound | 3.90 | 16.79 | 129 | Red |
| Example 52 | | 2-52 | | | | |
| Comparative | | Compound | 3.94 | 16.65 | 122 | Red |
| Example 53 | | 2-78 | | | | |
| Comparative | | Compound | 3.88 | 17.03 | 132 | Red |
| Example 54 | | 2-104 | | | | |
| Comparative | | Compound | 3.92 | 16.49 | 143 | Red |
| Example 55 | | 2-130 | | | | |
| Comparative | Compound | Compound | 3.89 | 16.47 | 144 | Red |
| Example 56 | A-12 | 2-21 | | | | |
| Comparative | | Compound | 3.95 | 16.68 | 122 | Red |
| Example 57 | | 2-49 | | | | |
| Comparative | | Compound | 3.90 | 16.75 | 134 | Red |
| Example 58 | | 2-73 | | | | |
| Comparative | | Compound | 3.91 | 17.07 | 128 | Red |
| Example 59 | | 2-102 | | | | |
| Comparative | | Compound | 3.90 | 17.19 | 148 | Red |
| Example 60 | | 2-130 | | | | |

TABLE 3

| | | | | | Lifetime | |
|---|---|---|---|---|---|---|
| | The First | TheS econd | Voltage | Efficiency | T95 | Luminescent |
| Division | Host | Host | (V) | (cd/A) | (hr) | Color |
| Comparative | Compound | Compound | 4.05 | 14.69 | 109 | Red |
| Example 61 | 1-1 | B-1 | | | | |
| Comparative | Compound | | 4.13 | 14.62 | 113 | Red |
| Example 62 | 1-7 | | | | | |
| Comparative | Compound | | 4.16 | 14.81 | 113 | Red |
| Example 63 | 1-16 | | | | | |
| Comparative | Compound | | 4.12 | 15.57 | 93 | Red |
| Example 64 | 1-28 | | | | | |
| Comparative | Compound | | 4.07 | 16.11 | 105 | Red |
| Example 65 | 1-35 | | | | | |
| Comparative | Compound | | 4.06 | 15.48 | 111 | Red |
| Example 66 | 1-43 | | | | | |
| Comparative | Compound | | 4.12 | 16.37 | 120 | Red |
| Example 67 | 1-18 | | | | | |
| Comparative | Compound | | 4.14 | 15.22 | 102 | Red |
| Example 68 | 1-40 | | | | | |
| Comparative | Compound | Compound | 4.15 | 16.87 | 121 | Red |
| Example 69 | 1-2 | B-2 | | | | |
| Comparative | Compound | | 4.09 | 16.44 | 131 | Red |
| Example 70 | 1-10 | | | | | |
| Comparative | Compound | | 4.08 | 16.56 | 141 | Red |
| Example 71 | 1-19 | | | | | |
| Comparative | Compound | | 4.07 | 16.82 | 131 | Red |
| Example 72 | 1-26 | | | | | |
| Comparative | Compound | | 4.09 | 16.84 | 122 | Red |
| Example 73 | 1-31 | | | | | |
| Comparative | Compound | | 4.09 | 16.65 | 124 | Red |
| Example 74 | 1-22 | | | | | |
| Comparative | Compound | | 4.15 | 16.77 | 142 | Red |
| Example 75 | 1-34 | | | | | |
| Comparative | Compound | | 4.05 | 17.20 | 145 | Red |
| Example 76 | 1-41 | | | | | |
| Comparative | Compound | Compound | 3.99 | 17.06 | 153 | Red |
| Example 77 | 1-3 | B-3 | | | | |
| Comparative | Compound | | 3.90 | 17.42 | 149 | Red |
| Example 78 | 1-12 | | | | | |
| Comparative | Compound | | 3.88 | 17.12 | 170 | Red |
| Example 79 | 1-24 | | | | | |
| Comparative | Compound | | 3.91 | 17.64 | 151 | Red |
| Example 80 | 1-37 | | | | | |
| Comparative | Compound | | 3.89 | 17.81 | 165 | Red |
| Example 81 | 1-42 | | | | | |
| Comparative | Compound | | 3.92 | 17.98 | 160 | Red |
| Example 82 | 1-9 | | | | | |
| Comparative | Compound | | 3.88 | 17.23 | 162 | Red |
| Example 83 | 1-18 | | | | | |
| Comparative | Compound | | 3.88 | 17.84 | 154 | Red |
| Example 84 | 1-30 | | | | | |
| Comparative | Compound | Compound | 3.90 | 17.48 | 166 | Red |
| Example 85 | 1-4 | B-4 | | | | |
| Comparative | Compound | | 3.92 | 17.70 | 145 | Red |
| Example 86 | 1-11 | | | | | |
| Comparative | Compound | | 3.92 | 17.32 | 160 | Red |
| Example 87 | 1-23 | | | | | |
| Comparative | Compound | | 3.90 | 17.04 | 149 | Red |
| Example 88 | 1-36 | | | | | |
| Comparative | Compound | | 3.92 | 17.96 | 159 | Red |
| Example 89 | 1-44 | | | | | |
| Comparative | Compound | | 3.90 | 16.96 | 151 | Red |
| Example 90 | 1-16 | | | | | |
| Comparative | Compound | | 3.89 | 17.73 | 151 | Red |
| Example 91 | 1-28 | | | | | |
| Comparative | Compound | | 3.93 | 16.94 | 154 | Red |
| Example 92 | 1-37 | | | | | |
| Comparative | Compound | Compound | 4.05 | 16.62 | 144 | Red |
| Example 93 | 1-5 | B-5 | | | | |
| Comparative | Compound | | 4.06 | 17.03 | 147 | Red |
| Example 94 | 1-14 | | | | | |
| Comparative | Compound | | 4.17 | 16.86 | 129 | Red |
| Example 95 | 1-20 | | | | | |
| Comparative | Compound | | 4.08 | 16.62 | 122 | Red |
| Example 96 | 1-33 | | | | | |
| Comparative | Compound | | 4.17 | 17.08 | 125 | Red |
| Example 97 | 1-45 | | | | | |
| Comparative | Compound | | 4.10 | 16.78 | 145 | Red |
| Example 98 | 1-16 | | | | | |
| Comparative | Compound | | 4.11 | 16.86 | 123 | Red |
| Example 99 | 1-28 | | | | | |
| Comparative | Compound | | 4.12 | 16.50 | 144 | Red |
| Example 100 | 1-39 | | | | | |
| Comparative | Compound | Compound | 3.93 | 17.14 | 159 | Red |
| Example 101 | 1-6 | B-6 | | | | |
| Comparative | Compound | | 3.90 | 17.95 | 164 | Red |
| Example 102 | 1-13 | | | | | |
| Comparative | Compound | | 3.93 | 17.23 | 159 | Red |
| Example 103 | 1-21 | | | | | |
| Comparative | Compound | | 3.88 | 17.80 | 159 | Red |
| Example 104 | 1-32 | | | | | |
| Comparative | Compound | | 3.89 | 17.02 | 164 | Red |
| Example 105 | 1-40 | | | | | |
| Comparative | Compound | | 3.92 | 17.03 | 167 | Red |
| Example 106 | 1-27 | | | | | |
| Comparative | Compound | | 3.91 | 17.63 | 149 | Red |
| Example 107 | 1-36 | | | | | |
| Comparative | Compound | | 3.89 | 17.21 | 165 | Red |
| Example 108 | 1-45 | | | | | |
| Comparative | Compound | Compound | 4.13 | 16.34 | 91 | Red |
| Example 109 | 1-7 | B-7 | | | | |
| Comparative | Compound | | 4.09 | 15.04 | 125 | Red |
| Example 110 | 1-16 | | | | | |
| Comparative | Compound | | 4.09 | 16.06 | 103 | Red |
| Example 111 | 1-25 | | | | | |
| Comparative | Compound | | 4.16 | 14.67 | 93 | Red |
| Example 112 | 1-34 | | | | | |
| Comparative | Compound | | 4.05 | 14.92 | 107 | Red |
| Example 113 | 1-46 | | | | | |
| Comparative | Compound | | 4.14 | 14.51 | 121 | Red |
| Example 114 | 1-10 | | | | | |
| Comparative | Compound | | 4.11 | 16.27 | 125 | Red |
| Example 115 | 1-31 | | | | | |
| Comparative | Compound | | 4.05 | 16.52 | 125 | Red |
| Example 116 | 1-42 | | | | | |
| Comparative | Compound | Compound | 3.95 | 16.70 | 128 | Red |
| Example 117 | 1-8 | B-8 | | | | |
| Comparative | Compound | | 3.91 | 16.77 | 122 | Red |
| Example 118 | 1-17 | | | | | |
| Comparative | Compound | | 3.92 | 16.56 | 133 | Red |
| Example 119 | 1-29 | | | | | |
| Comparative | Compound | | 3.95 | 16.88 | 122 | Red |
| Example 120 | 1-38 | | | | | |
| Comparative | Compound | | 3.91 | 17.20 | 138 | Red |
| Example 121 | 1-12 | | | | | |
| Comparative | Compound | | 3.90 | 17.01 | 126 | Red |
| Example 122 | 1-21 | | | | | |
| Comparative | Compound | | 3.90 | 16.53 | 135 | Red |
| Example 123 | 1-33 | | | | | |
| Comparative | Compound | | 3.91 | 16.73 | 141 | Red |
| Example 124 | 1-45 | | | | | |
| Comparative | Compound | Compound | 3.93 | 17.64 | 160 | Red |
| Example 125 | 1-9 | B-9 | | | | |
| Comparative | Compound | | 3.95 | 17.58 | 170 | Red |
| Example 126 | 1-18 | | | | | |
| Comparative | Compound | | 3.88 | 17.84 | 157 | Red |
| Example 127 | 1-22 | | | | | |
| Comparative | Compound | | 3.94 | 17.82 | 163 | Red |
| Example 128 | 1-30 | | | | | |
| Comparative | Compound | | 3.89 | 17.13 | 162 | Red |
| Example 129 | 1-41 | | | | | |
| Comparative | Compound | | 3.94 | 17.18 | 154 | Red |
| Example 130 | 1-12 | | | | | |
| Comparative | Compound | | 3.89 | 17.16 | 160 | Red |
| Example 131 | 1-26 | | | | | |
| Comparative | Compound | | 3.90 | 17.15 | 168 | Red |
| Example 132 | 1-37 | | | | | |
| Comparative | Compound | Compound | 3.88 | 17.24 | 160 | Red |
| Example 133 | 1-1 | B-10 | | | | |
| Comparative | Compound | | 3.91 | 17.49 | 148 | Red |
| Example 134 | 1-15 | | | | | |
| Comparative | Compound | | 3.95 | 17.33 | 169 | Red |
| Example 135 | 1-26 | | | | | |
| Comparative | Compound | | 3.94 | 17.33 | 165 | Red |
| Example 136 | 1-35 | | | | | |
| Comparative | Compound | | 3.88 | 17.74 | 149 | Red |
| Example 137 | 1-21 | | | | | |
| Comparative | Compound | | 3.90 | 16.92 | 159 | Red |
| Example 138 | 1-30 | | | | | |
| Comparative | Compound | | 3.89 | 17.44 | 163 | Red |
| Example 139 | 1-42 | | | | | |
| Comparative | Compound | | 3.95 | 17.18 | 149 | Red |
| Example 140 | 1-47 | | | | | |
| Comparative | Compound | Compound | 4.06 | 16.45 | 139 | Red |
| Example 141 | 1-3 | B-11 | | | | |
| Comparative | Compound | | 4.16 | 16.96 | 140 | Red |
| Example 142 | 1-14 | | | | | |
| Comparative | Compound | | 4.07 | 17.19 | 125 | Red |
| Example 143 | 1-27 | | | | | |
| Comparative | Compound | | 4.12 | 16.58 | 141 | Red |
| Example 144 | 1-39 | | | | | |
| Comparative | Compound | | 4.17 | 16.44 | 135 | Red |
| Example 145 | 1-47 | | | | | |
| Comparative | Compound | | 4.13 | 16.55 | 139 | Red |
| Example 146 | 1-9 | | | | | |
| Comparative | Compound | | 4.05 | 17.01 | 142 | Red |
| Example 147 | 1-18 | | | | | |
| Comparative | Compound | | 4.06 | 16.84 | 148 | Red |
| Example 148 | 1-31 | | | | | |
| Comparative | Compound | Compound | 3.92 | 17.10 | 160 | Red |
| Example 149 | 1-7 | B-12 | | | | |
| Comparative | Compound | | 3.89 | 17.14 | 166 | Red |
| Example 150 | 1-16 | | | | | |
| Comparative | Compound | | 3.91 | 17.35 | 160 | Red |
| Example 151 | 1-25 | | | | | |
| Comparative | Compound | | 3.94 | 17.24 | 160 | Red |
| Example 152 | 1-34 | | | | | |
| Comparative | Compound | | 3.95 | 17.00 | 156 | Red |
| Example 153 | 1-46 | | | | | |
| Comparative | Compound | | 3.93 | 17.93 | 153 | Red |
| Example 154 | 1-10 | | | | | |
| Comparative | Compound | | 3.93 | 17.32 | 151 | Red |
| Example 155 | 1-31 | | | | | |
| Comparative | Compound | | 3.89 | 17.33 | 164 | Red |
| Example 156 | 1-42 | | | | | |

When a current was applied to the organic light emitting devices manufactured in Examples 1 to 340 and Comparative Examples 1 to 156, the results of Tables 1 to 3 above were obtained. The red organic light emitting devices of Examples and Comparative Examples used the materials used widely in the prior art, and are structured to use Compound EB-1 as an electron blocking layer and to use Dp-7 as a dopant of the red light emitting layer. As shown in Table 2, when Examples Compounds A-1 to A-12 of Comparative Examples and the compound represented by Chemical Formula 2 of the present disclosure were co-deposited and used as a red light emitting layer, the results usually showed that the driving voltage increased and the efficiency and lifetime decreased as compared with the combination of the present disclosure. As shown in Table 3, even when Compounds B-1 to B-12 of Comparative Examples and the compound represented by Chemical Formula 1 of the present disclosure were co-deposited and used as a red light emitting layer, the results showed that the driving voltage increased and the efficiency and lifespan decreased.

In light of these result, it can be inferred that the reason why the driving voltage is improved and the efficiency and lifespan are increased is because the combination of the compound of Compound Formula 1 as the first host and the compound of Compound Formula 2 as the second host of the present disclosure facilitates energy transfer to the red dopant in the red light emitting layer.

Therefore, it can be confirmed that since the combination of the compound represented by Chemical Formula 1 and the compound represented by Chemical Formula 2 of the present disclosure has a more stable balance in the light emitting layer than the combination with the compounds of Comparative Examples, electrons and holes combine to form excitons, thereby greatly increasing efficiency and lifetime. From these facts, it was confirmed that when the compound represented by Chemical Formula 1 and the compound represented by Chemical Formula 2 of the present disclosure are co-deposited and used as the host of the red light emitting layer, the driving voltage, luminous efficiency and lifespan characteristics of organic light emitting devices can be improved.

### DESCRIPTION OF SYMBOLS

1: substrate
2: anode
3: light emitting layer
4: cathode
5: hole injection layer
6: hole transport layer
7: electron blocking layer
8: hole blocking layer
9: electron injection and transport layer

1. An organic light emitting device comprising:
an anode;
a cathode; and
a light emitting layer between the anode and the cathode,
wherein the light emitting layer includes a compound represented by the following Chemical Formula 1 and a compound represented by the following Chemical Formula 2: 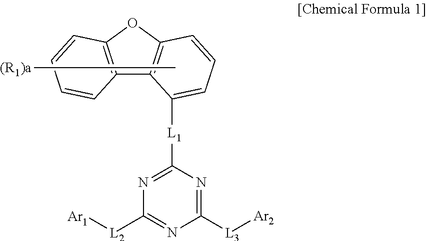
in Chemical Formula 1,
Ar₁ and Ar₂ are each independently a substituted or unsubstituted C₆₋₆₀ aryl; or a substituted or unsubstituted C₂₋₆₀ heteroaryl containing one or more selected from the group consisting of N, O and S,
L₁ is a single bond or one selected from the group consisting of 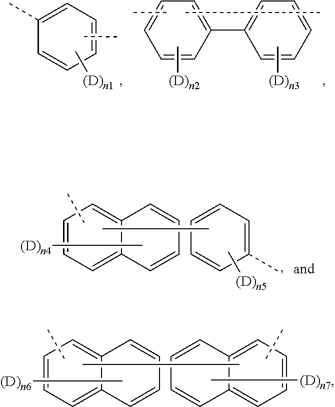
wherein D is deuterium, n1 to n3 and n5 are each independently an integer of 0 to 4, and n4, n6 and n7 are each independently an integer of 0 to 6,
L₂ and L₃ are each independently a single bond; or a substituted or unsubstituted C₆₋₆₀ arylene,
R₁ is each independently hydrogen; deuterium; a substituted or unsubstituted C₆₋₆₀ aryl; or a substituted or unsubstituted C₂₋₆₀ heteroaryl containing one or more selected from the group consisting of N, O and S, with the proviso that at least one R₁ is deuterium, phenyl substituted with deuterium, a substituted or unsubstituted C₁₀₋₆₀ aryl or a substituted or unsubstituted C₂₋₆₀ heteroaryl containing one or more selected from the group consisting of N, O and S, and
a is an integer of 1 to 7,
wherein the compound represented by Chemical Formula 1 optionally contain at least one deuterium, 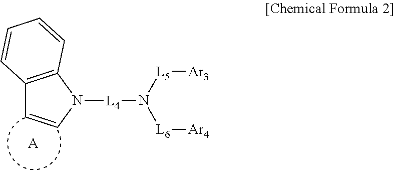
in Chemical Formula 2,
A is a benzene ring or a naphthalene ring,
Ar₃ and Ar₄ are each independently a substituted or unsubstituted C₆₋₆₀ aryl; or a substituted or unsubstituted C₂₋₆₀ heteroaryl containing one or more selected from the group consisting of N, O and S,
L₄ is a substituted or unsubstituted C₆₋₆₀ arylene, and
L₅ and L₆ are each independently a single bond, a substituted or unsubstituted C₆₋₆₀ arylene, or a substituted or unsubstituted C₂₋₆₀ heteroarylene containing one or more selected from the group consisting of N, O and S.

2. The organic light emitting device according to claim 1, wherein
Ar₁ and Ar₂ are each independently phenyl, biphenylyl, terphenylyl, naphthyl, phenanthrenyl, dibenzofuranyl, dibenzothiophenyl, or triphenylsilyl phenyl,
each of which is unsubstituted or substituted with deuterium.

3. The organic light emitting device according to claim 1, wherein
L₂ and L₃ are each independently a single bond, phenylene, biphenyldiyl, or naphthalenediyl,
each of which, except for a single bond, is unsubstituted or substituted with deuterium.

4. The organic light emitting device according to claim 1, wherein
R₁ is each independently hydrogen, deuterium, phenyl, biphenylyl, terphenylyl, naphthyl, phenanthrenyl, triphenylenyl, fluoranthenyl, naphthyl phenyl, phenyl naphthyl, dihydroindenyl, dibenzofuranyl, dibenzothiophenyl, benzonaphthofuranyl, or benzonaphthothiophenyl, with the proviso that at least one R₁ is deuterium, phenyl substituted with deuterium, biphenylyl, terphenylyl, naphthyl, phenanthrenyl, triphenylenyl, fluoranthenyl, naphthyl phenyl, phenyl naphthyl, dibenzofuranyl, dibenzothiophenyl, benzonaphthofuranyl, or benzonaphthothiophenyl, each of which, except for deuterium and phenyl substituted with deuterium, is
substituted or substituted with deuterium.

5. The organic light emitting device according to claim 1, wherein
the compound represented by Chemical Formula 1 is one selected from the following:

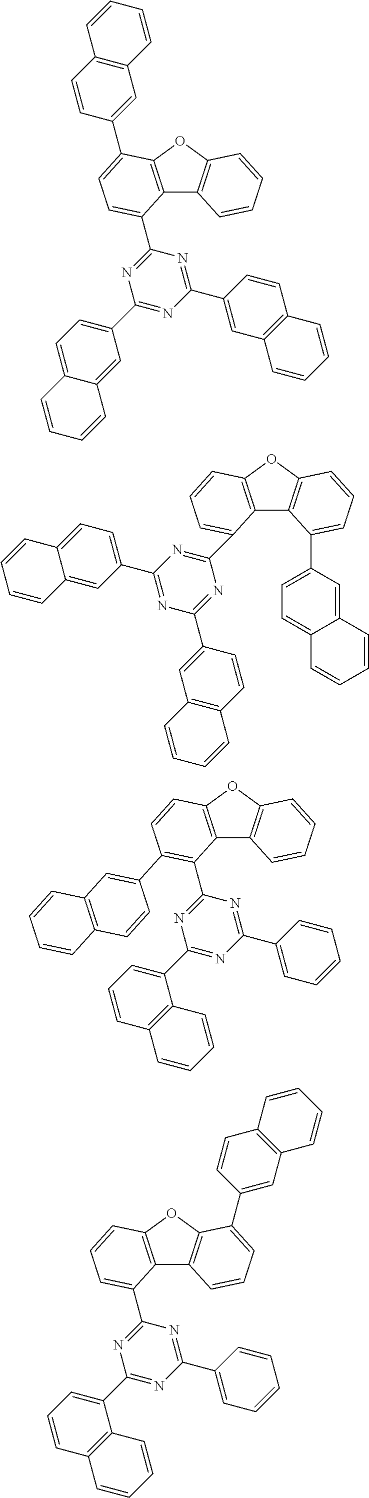

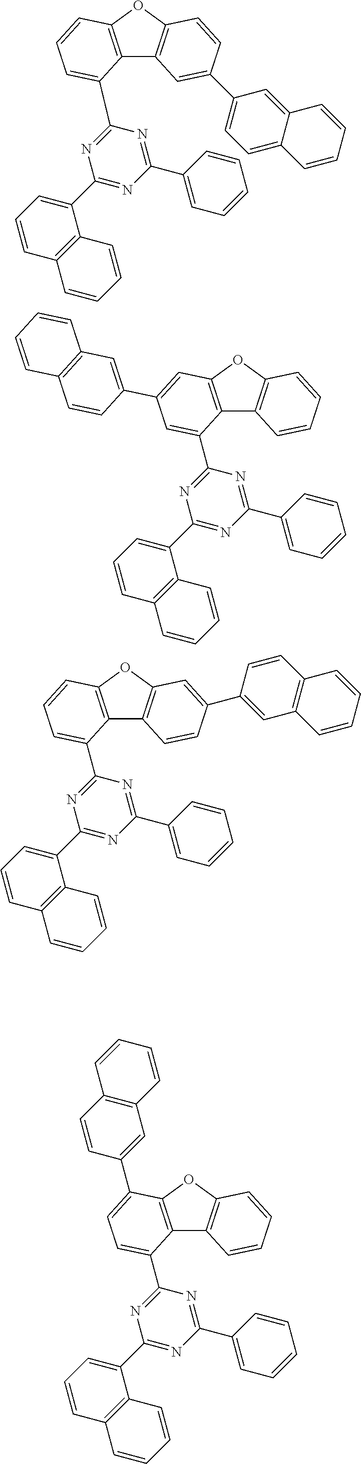

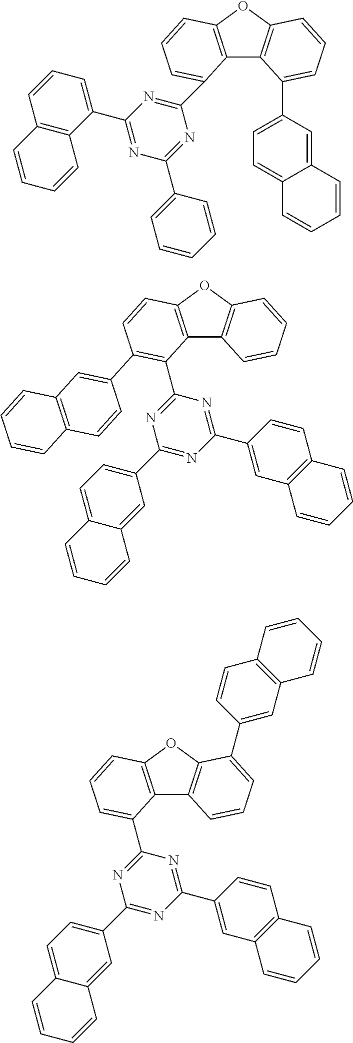

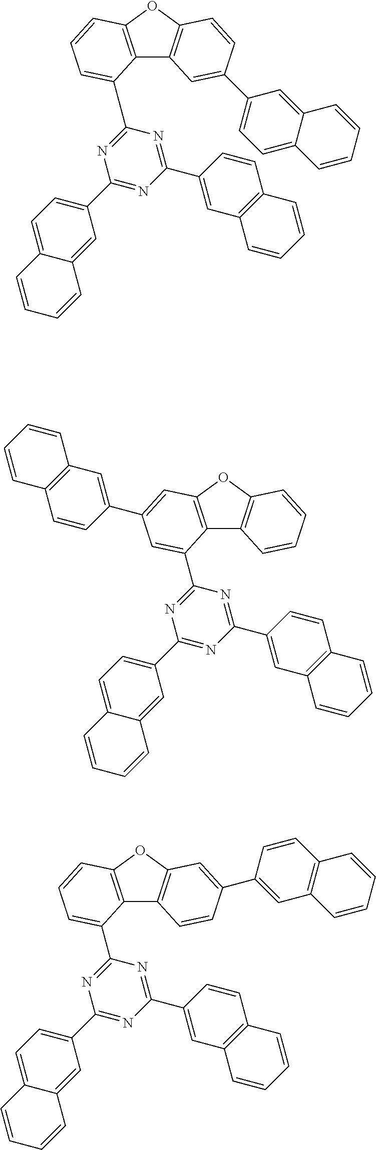 [SMILES: c1ccc2cc(-c3ccc4oc5cccc(-c6nc(-c7ccc8ccccc8c7)nc(-c7ccc8ccccc8c7)n6)c5c4c3)ccc2c1 ; c1ccc2cc(-c3cc(-c4nc(-c5ccc6ccccc6c5)nc(-c5ccc6ccccc6c5)n4)c4c(c3)oc3ccccc34)ccc2c1 ; c1ccc2cc(-c3ccc4c(c3)oc3cccc(-c5nc(-c6ccc7ccccc7c6)nc(-c6ccc7ccccc7c6)n5)c34)ccc2c1]

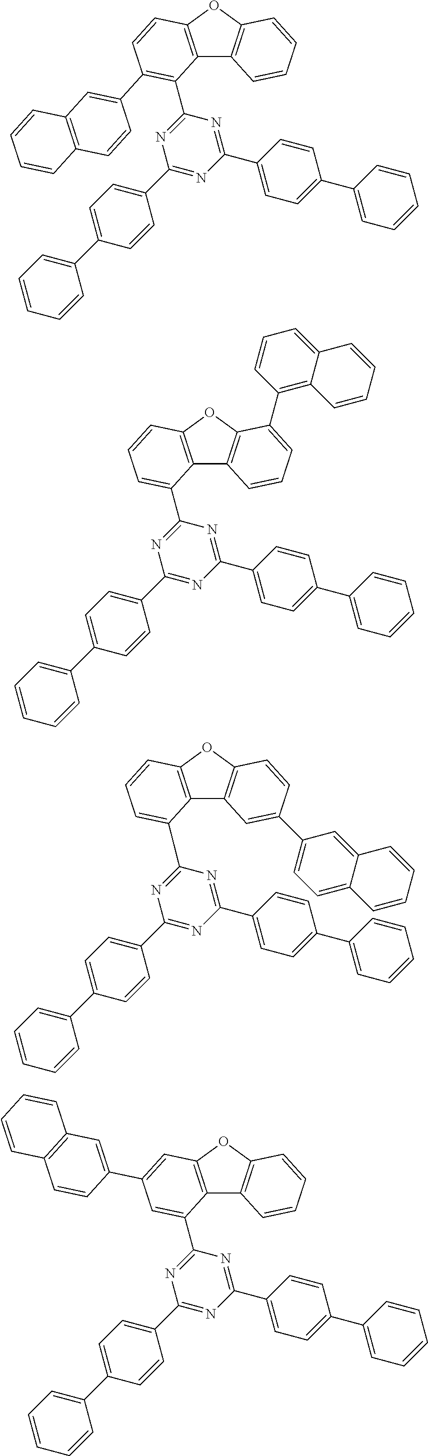

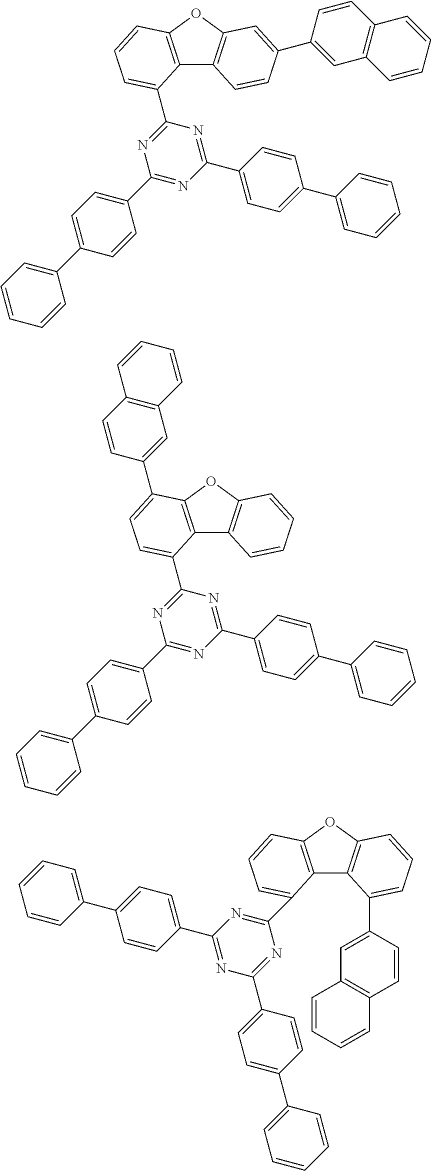 [SMILES: c1ccc(-c2ccc(-c3nc(-c4ccc(-c5ccccc5)cc4)nc(-c4cccc5oc6cc(-c7ccc8ccccc8c7)ccc6c45)n3)cc2)cc1 ; c1ccc(-c2ccc(-c3nc(-c4ccc(-c5ccccc5)cc4)nc(-c4ccc(-c5ccc6ccccc6c5)c5oc6ccccc6c45)n3)cc2)cc1 ; c1ccc(-c2ccc(-c3nc(-c4ccc(-c5ccccc5)cc4)nc(-c4cccc5oc6cccc(-c7ccc8ccccc8c7)c6c45)n3)cc2)cc1]

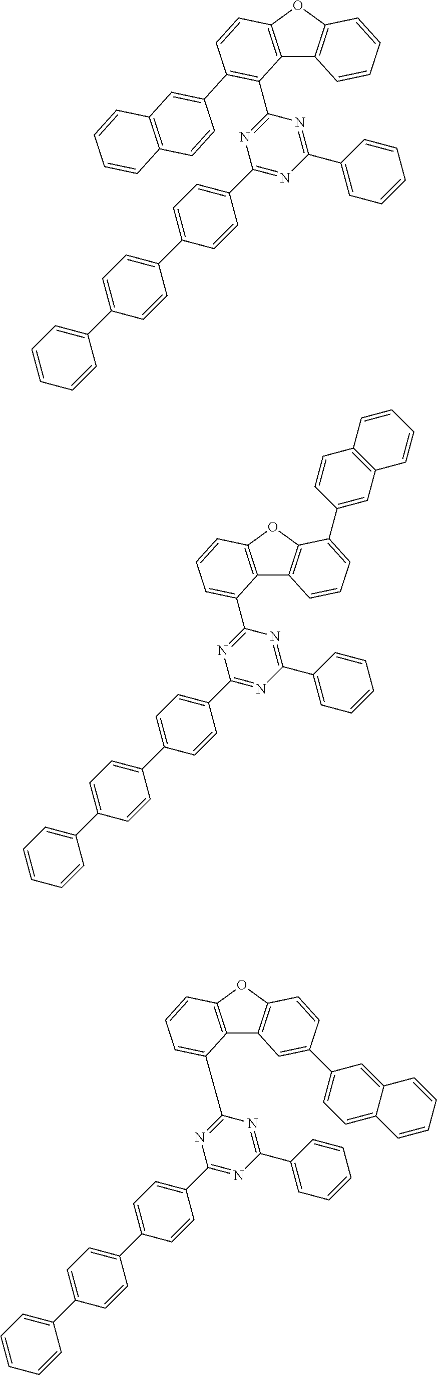 [SMILES: c1ccc(-c2ccc(-c3ccc(-c4nc(-c5ccccc5)nc(-c5c(-c6ccc7ccccc7c6)ccc6oc7ccccc7c56)n4)cc3)cc2)cc1 ; c1ccc(-c2ccc(-c3ccc(-c4nc(-c5ccccc5)nc(-c5cccc6oc7c(-c8ccc9ccccc9c8)cccc7c56)n4)cc3)cc2)cc1 ; c1ccc(-c2ccc(-c3ccc(-c4nc(-c5ccccc5)nc(-c5cccc6oc7ccc(-c8ccc9ccccc9c8)cc7c56)n4)cc3)cc2)cc1]

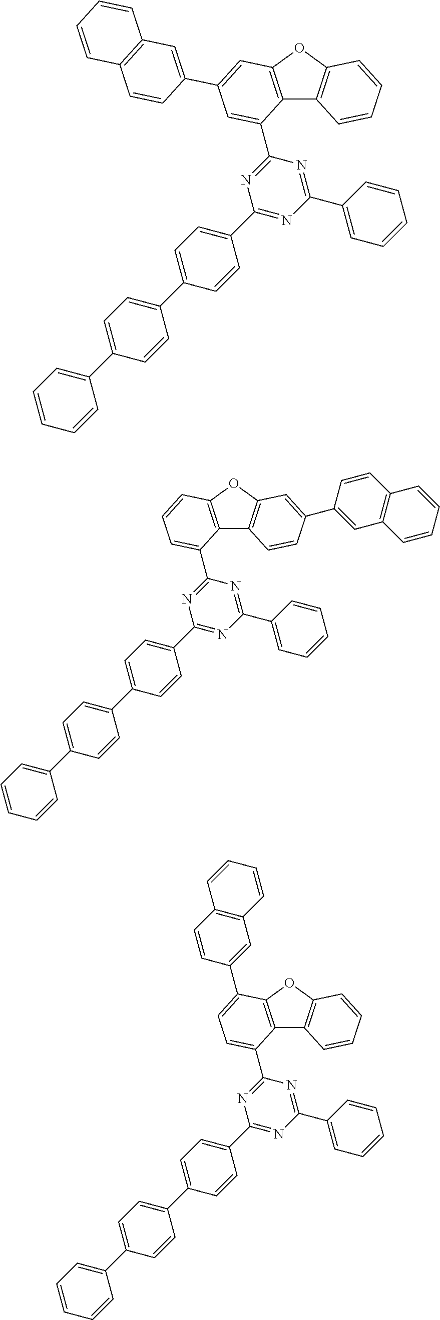 [SMILES: c1ccc(-c2ccc(-c3ccc(-c4nc(-c5ccccc5)nc(-c5cc(-c6ccc7ccccc7c6)cc6oc7ccccc7c56)n4)cc3)cc2)cc1 ; c1ccc(-c2ccc(-c3ccc(-c4nc(-c5ccccc5)nc(-c5cccc6oc7cc(-c8ccc9ccccc9c8)ccc7c56)n4)cc3)cc2)cc1 ; c1ccc(-c2ccc(-c3ccc(-c4nc(-c5ccccc5)nc(-c5ccc(-c6ccc7ccccc7c6)c6oc7ccccc7c56)n4)cc3)cc2)cc1]

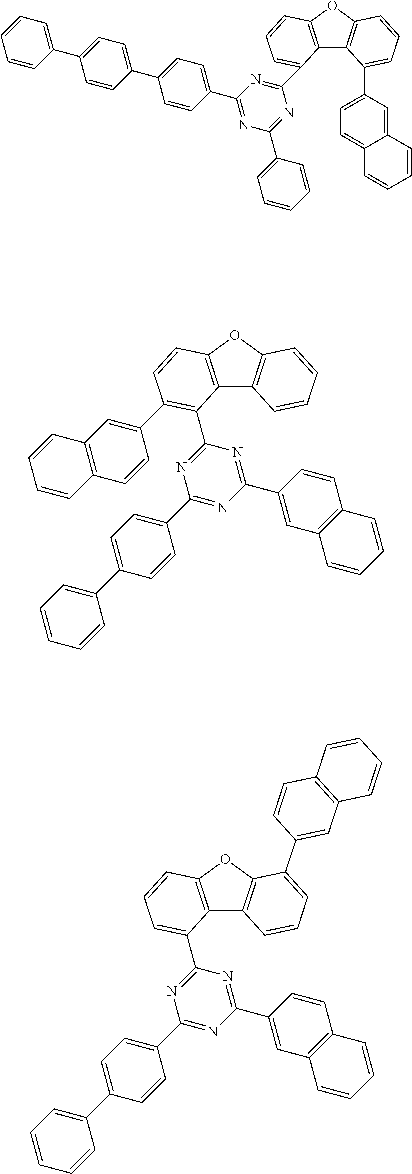 [SMILES: c1ccc(-c2ccc(-c3ccc(-c4nc(-c5ccccc5)nc(-c5cccc6oc7cccc(-c8ccc9ccccc9c8)c7c56)n4)cc3)cc2)cc1 ; c1ccc(-c2ccc(-c3nc(-c4ccc5ccccc5c4)nc(-c4c(-c5ccc6ccccc6c5)ccc5oc6ccccc6c45)n3)cc2)cc1 ; c1ccc(-c2ccc(-c3nc(-c4ccc5ccccc5c4)nc(-c4cccc5oc6c(-c7ccc8ccccc8c7)cccc6c45)n3)cc2)cc1]

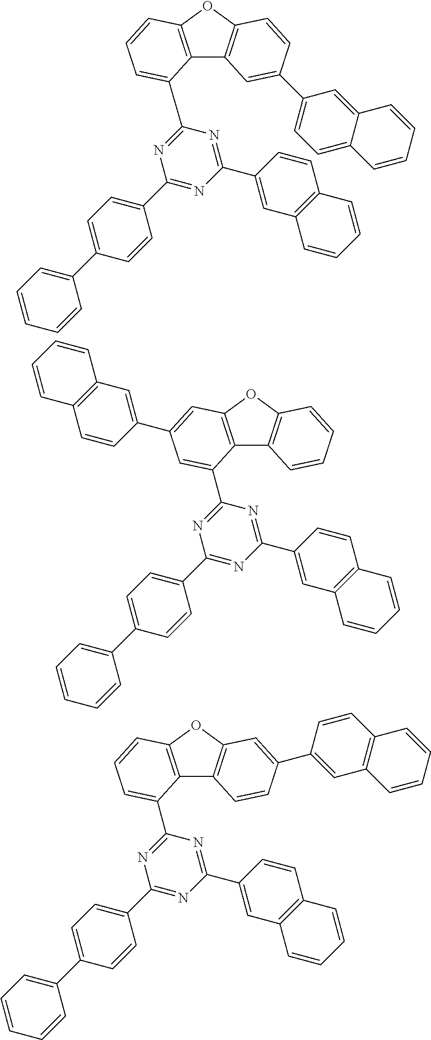

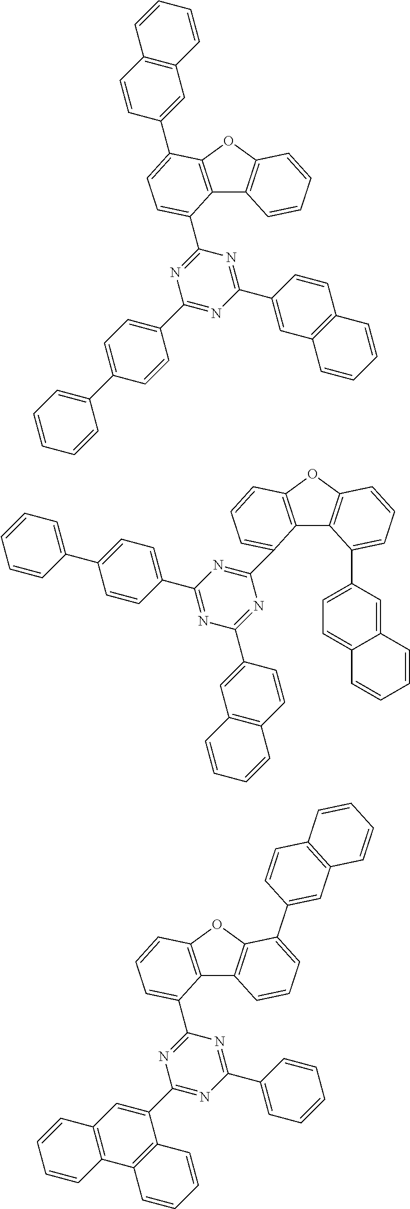 [SMILES: c1ccc(-c2ccc(-c3nc(-c4ccc5ccccc5c4)nc(-c4ccc(-c5ccc6ccccc6c5)c5oc6ccccc6c45)n3)cc2)cc1 ; c1ccc(-c2ccc(-c3nc(-c4ccc5ccccc5c4)nc(-c4cccc5oc6cccc(-c7ccc8ccccc8c7)c6c45)n3)cc2)cc1 ; c1ccc(-c2nc(-c3cc4ccccc4c4ccccc34)nc(-c3cccc4oc5c(-c6ccc7ccccc7c6)cccc5c34)n2)cc1]

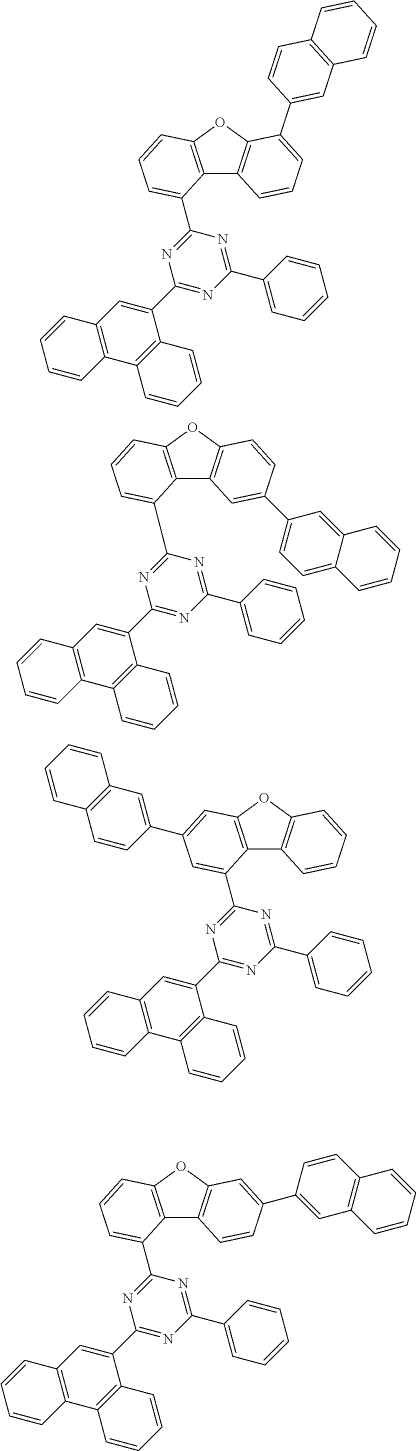

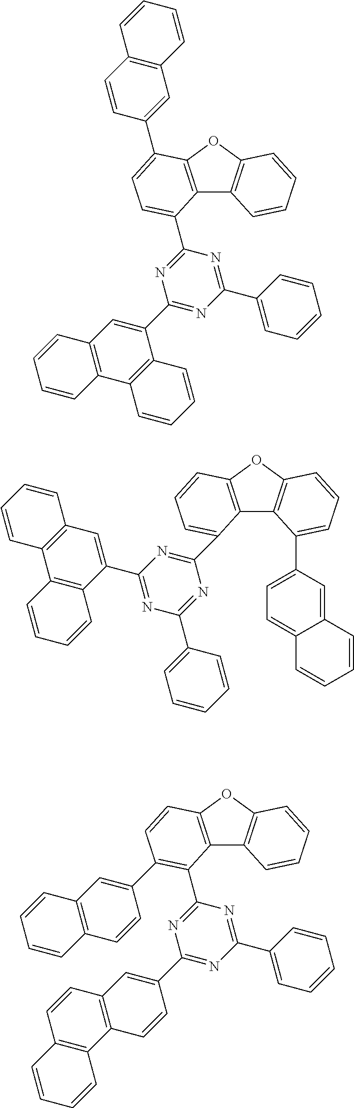 [SMILES: c1ccc(-c2nc(-c3cc4ccccc4c4ccccc34)nc(-c3ccc(-c4ccc5ccccc5c4)c4oc5ccccc5c34)n2)cc1 ; c1ccc(-c2nc(-c3cc4ccccc4c4ccccc34)nc(-c3cccc4oc5cccc(-c6ccc7ccccc7c6)c5c34)n2)cc1 ; c1ccc(-c2nc(-c3ccc4c(ccc5ccccc54)c3)nc(-c3c(-c4ccc5ccccc5c4)ccc4oc5ccccc5c34)n2)cc1]

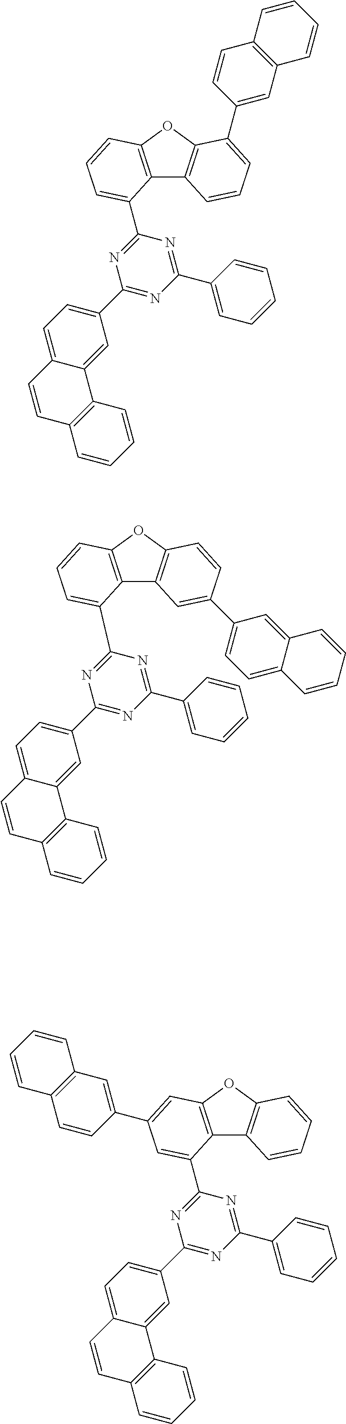 [SMILES: c1ccc(-c2nc(-c3ccc4ccc5ccccc5c4c3)nc(-c3cccc4oc5c(-c6ccc7ccccc7c6)cccc5c34)n2)cc1 ; c1ccc(-c2nc(-c3ccc4ccc5ccccc5c4c3)nc(-c3cccc4oc5ccc(-c6ccc7ccccc7c6)cc5c34)n2)cc1 ; c1ccc(-c2nc(-c3ccc4ccc5ccccc5c4c3)nc(-c3cc(-c4ccc5ccccc5c4)cc4oc5ccccc5c34)n2)cc1]

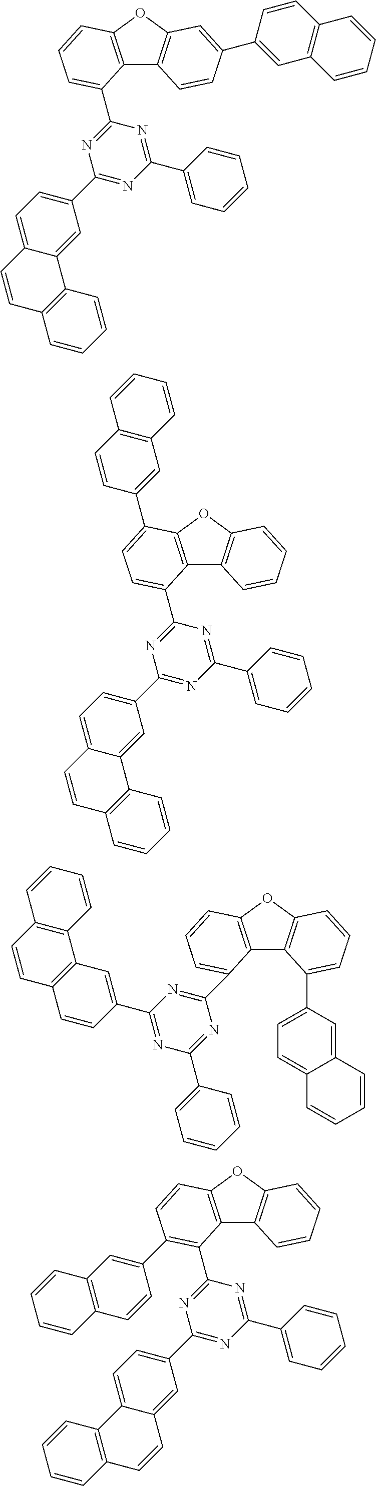

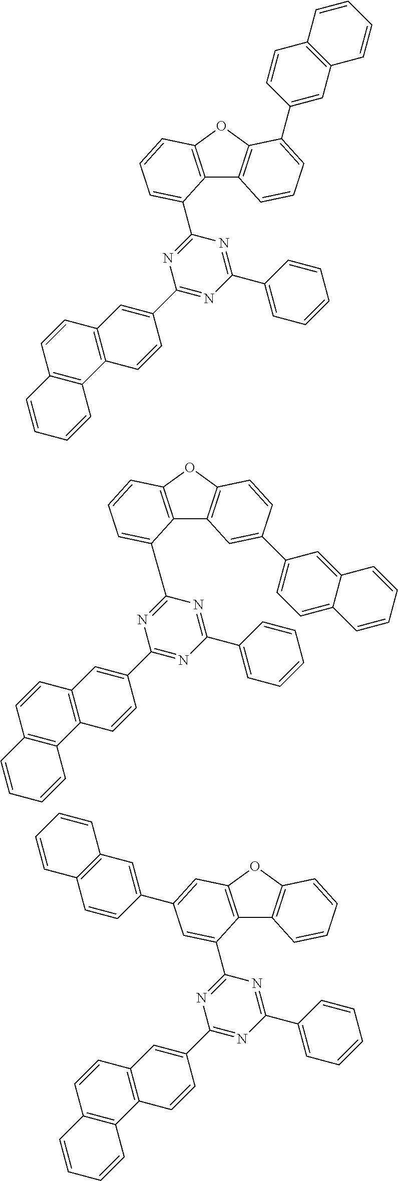

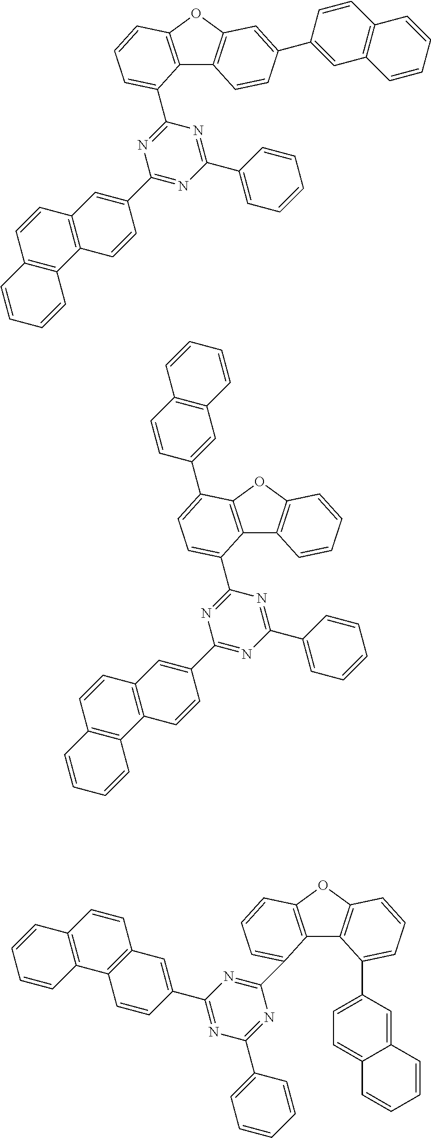 [SMILES: c1ccc(-c2nc(-c3ccc4c(ccc5ccccc54)c3)nc(-c3cccc4oc5cc(-c6ccc7ccccc7c6)ccc5c34)n2)cc1 ; c1ccc(-c2nc(-c3ccc4c(ccc5ccccc54)c3)nc(-c3ccc(-c4ccc5ccccc5c4)c4oc5ccccc5c34)n2)cc1 ; c1ccc(-c2nc(-c3ccc4c(ccc5ccccc54)c3)nc(-c3cccc4oc5cccc(-c6ccc7ccccc7c6)c5c34)n2)cc1]

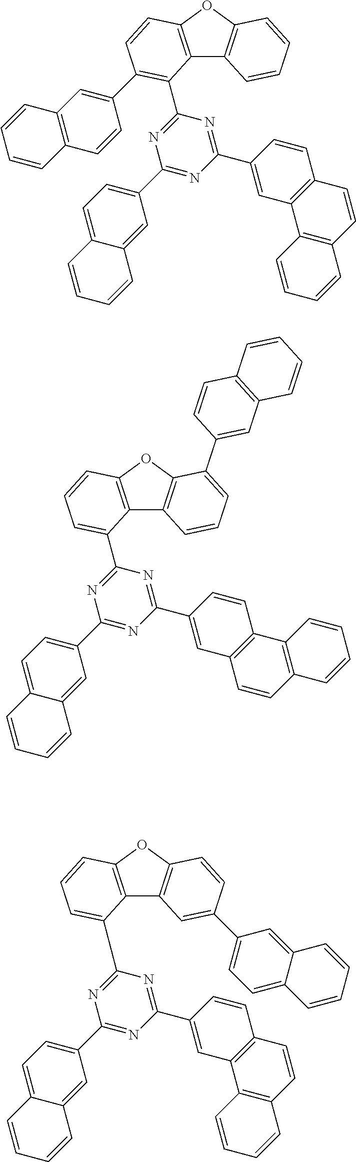

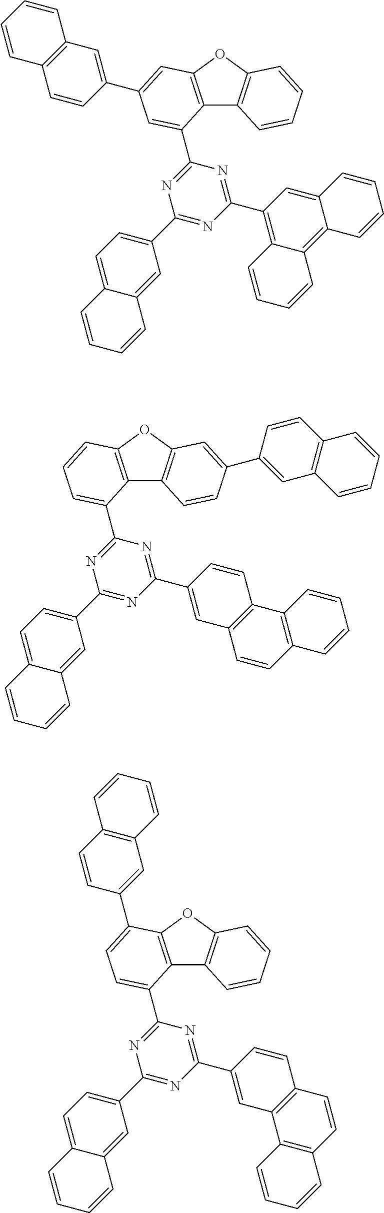 [SMILES: c1ccc2cc(-c3cc(-c4nc(-c5ccc6ccccc6c5)nc(-c5cc6ccccc6c6ccccc56)n4)c4c(c3)oc3ccccc34)ccc2c1 ; c1ccc2cc(-c3ccc4c(c3)oc3cccc(-c5nc(-c6ccc7ccccc7c6)nc(-c6ccc7c(ccc8ccccc87)c6)n5)c34)ccc2c1 ; c1ccc2cc(-c3nc(-c4ccc5ccc6ccccc6c5c4)nc(-c4ccc(-c5ccc6ccccc6c5)c5oc6ccccc6c45)n3)ccc2c1]

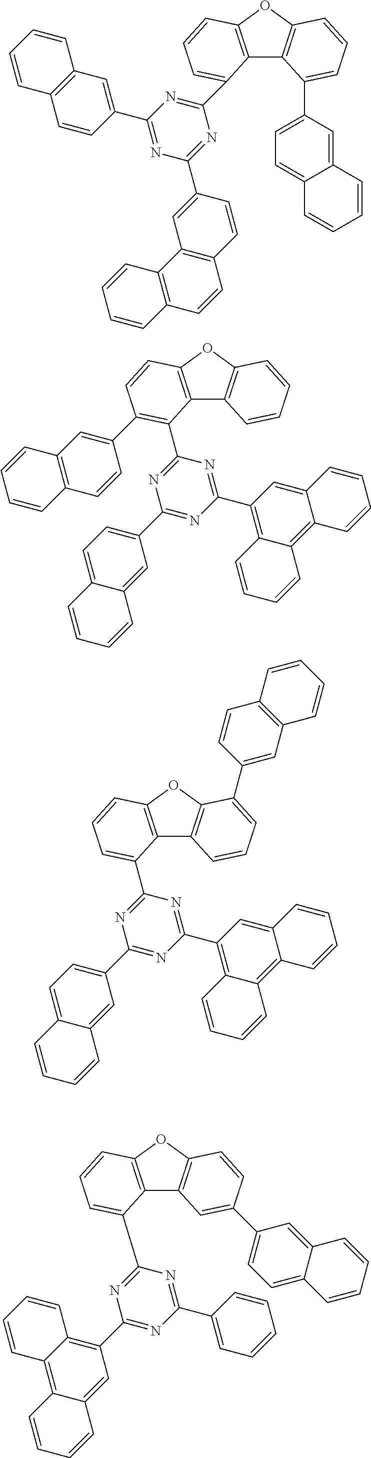

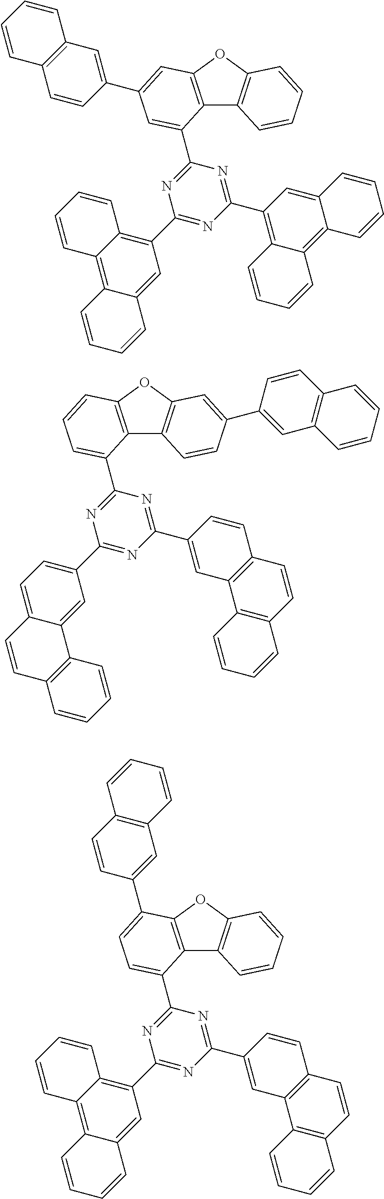

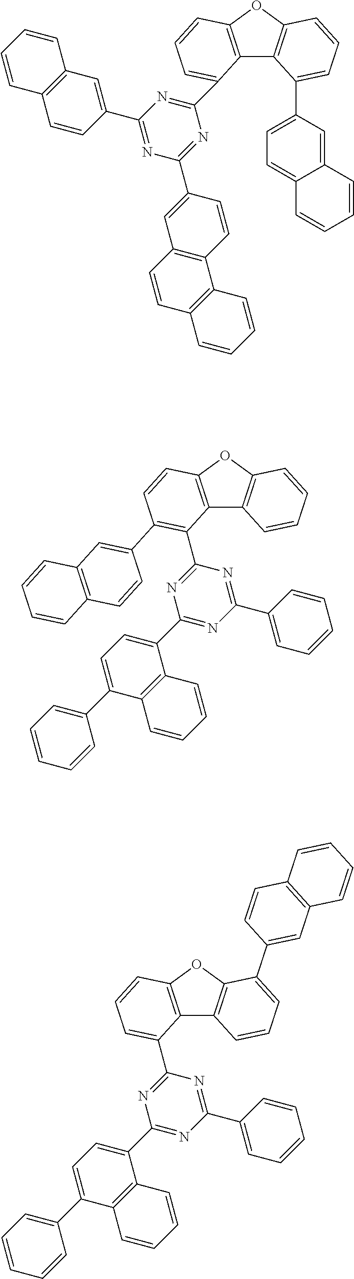 [SMILES: c1ccc2cc(-c3nc(-c4ccc5c(ccc6ccccc65)c4)nc(-c4cccc5oc6cccc(-c7ccc8ccccc8c7)c6c45)n3)ccc2c1 ; c1ccc(-c2nc(-c3ccc(-c4ccccc4)c4ccccc34)nc(-c3c(-c4ccc5ccccc5c4)ccc4oc5ccccc5c34)n2)cc1 ; c1ccc(-c2nc(-c3ccc(-c4ccccc4)c4ccccc34)nc(-c3cccc4oc5c(-c6ccc7ccccc7c6)cccc5c34)n2)cc1]

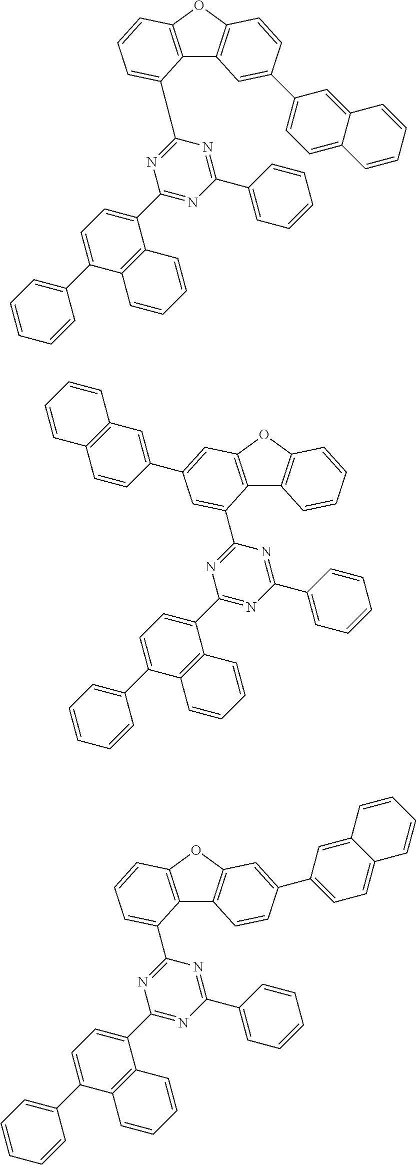

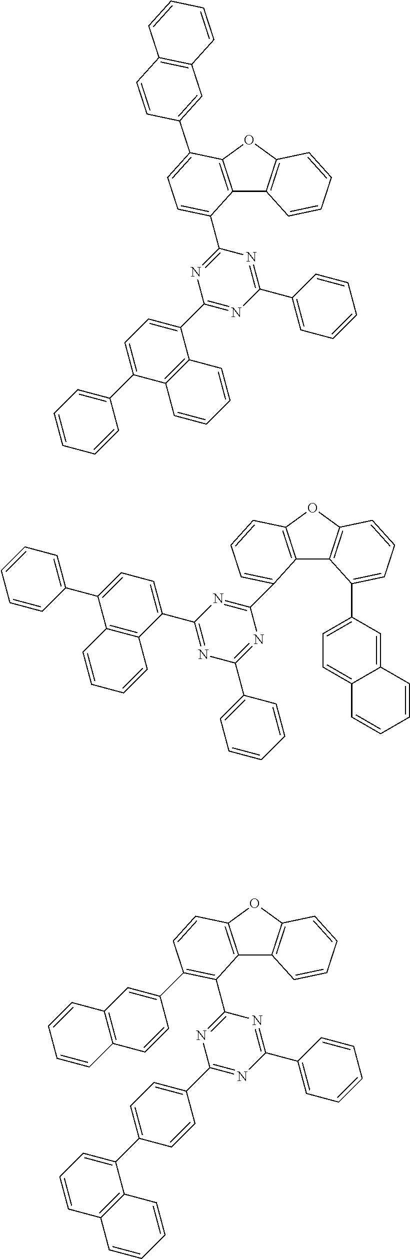 [SMILES: c1ccc(-c2nc(-c3ccc(-c4ccccc4)c4ccccc34)nc(-c3ccc(-c4ccc5ccccc5c4)c4oc5ccccc5c34)n2)cc1 ; c1ccc(-c2nc(-c3ccc(-c4ccccc4)c4ccccc34)nc(-c3cccc4oc5cccc(-c6ccc7ccccc7c6)c5c34)n2)cc1 ; c1ccc(-c2nc(-c3ccc(-c4cccc5ccccc45)cc3)nc(-c3c(-c4ccc5ccccc5c4)ccc4oc5ccccc5c34)n2)cc1]

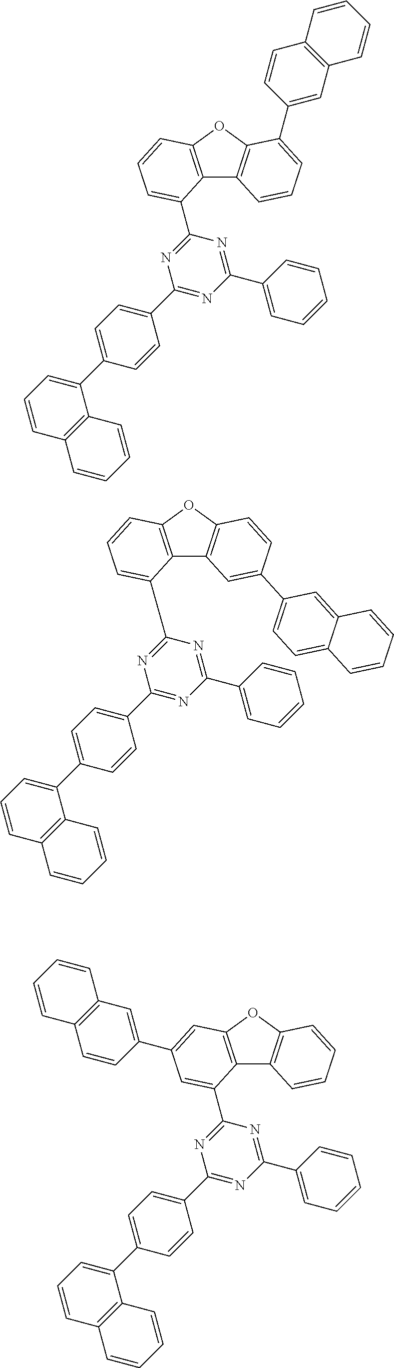

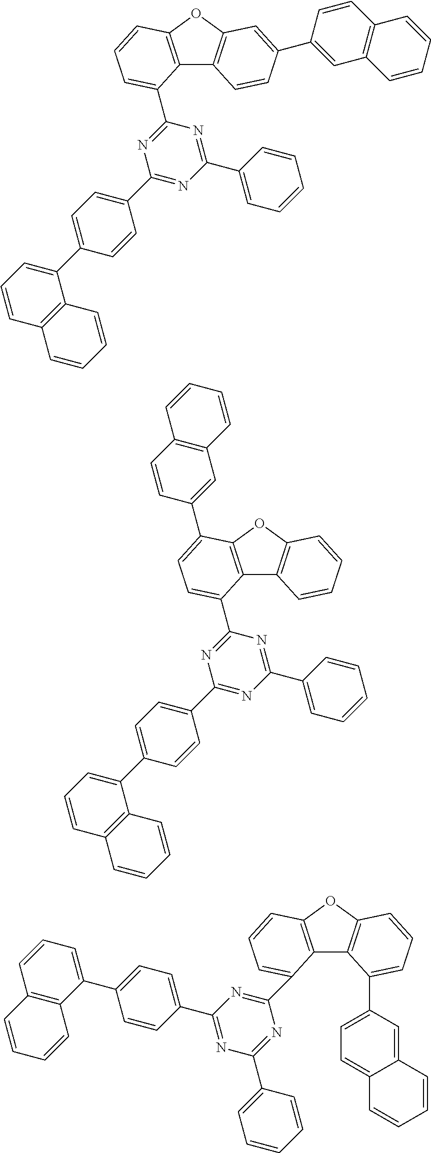 [SMILES: c1ccc(-c2nc(-c3ccc(-c4cccc5ccccc45)cc3)nc(-c3cccc4oc5cc(-c6ccc7ccccc7c6)ccc5c34)n2)cc1 ; c1ccc(-c2nc(-c3ccc(-c4cccc5ccccc45)cc3)nc(-c3ccc(-c4ccc5ccccc5c4)c4oc5ccccc5c34)n2)cc1 ; c1ccc(-c2nc(-c3ccc(-c4cccc5ccccc45)cc3)nc(-c3cccc4oc5cccc(-c6ccc7ccccc7c6)c5c34)n2)cc1]

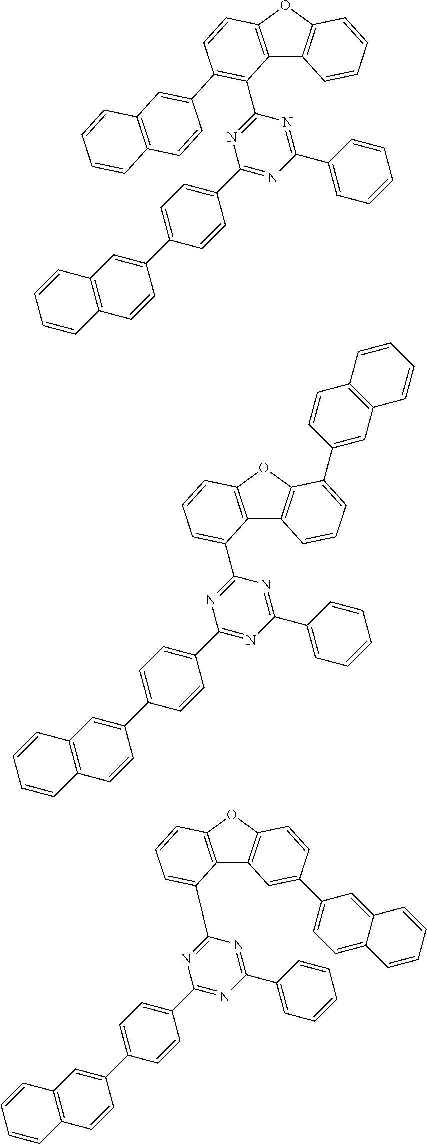 [SMILES: c1ccc(-c2nc(-c3ccc(-c4ccc5ccccc5c4)cc3)nc(-c3c(-c4ccc5ccccc5c4)ccc4oc5ccccc5c34)n2)cc1 ; c1ccc(-c2nc(-c3ccc(-c4ccc5ccccc5c4)cc3)nc(-c3cccc4oc5c(-c6ccc7ccccc7c6)cccc5c34)n2)cc1 ; c1ccc(-c2nc(-c3ccc(-c4ccc5ccccc5c4)cc3)nc(-c3cccc4oc5ccc(-c6ccc7ccccc7c6)cc5c34)n2)cc1]

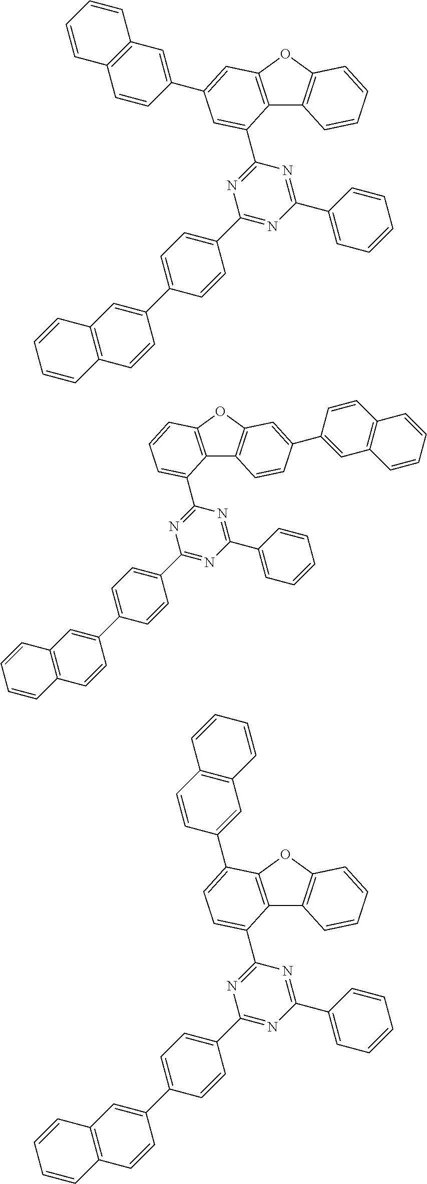 [SMILES: c1ccc(-c2nc(-c3ccc(-c4ccc5ccccc5c4)cc3)nc(-c3cc(-c4ccc5ccccc5c4)cc4oc5ccccc5c34)n2)cc1 ; c1ccc(-c2nc(-c3ccc(-c4ccc5ccccc5c4)cc3)nc(-c3cccc4oc5cc(-c6ccc7ccccc7c6)ccc5c34)n2)cc1 ; c1ccc(-c2nc(-c3ccc(-c4ccc5ccccc5c4)cc3)nc(-c3ccc(-c4ccc5ccccc5c4)c4oc5ccccc5c34)n2)cc1]

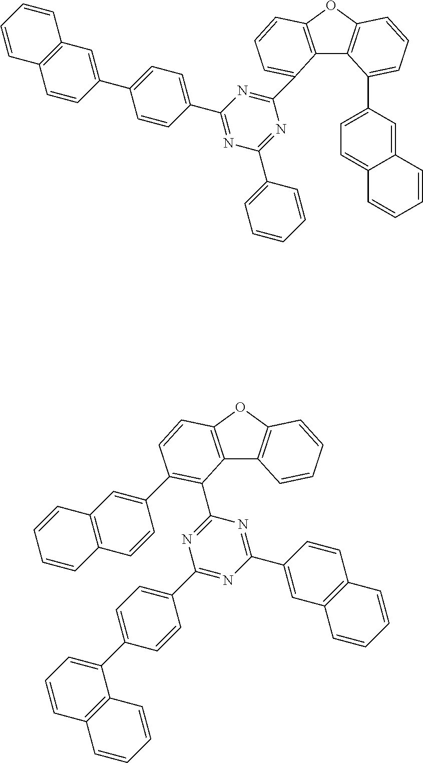 [SMILES: c1ccc(-c2nc(-c3ccc(-c4ccc5ccccc5c4)cc3)nc(-c3cccc4oc5cccc(-c6ccc7ccccc7c6)c5c34)n2)cc1 ; c1ccc2cc(-c3nc(-c4ccc(-c5cccc6ccccc56)cc4)nc(-c4c(-c5ccc6ccccc6c5)ccc5oc6ccccc6c45)n3)ccc2c1]

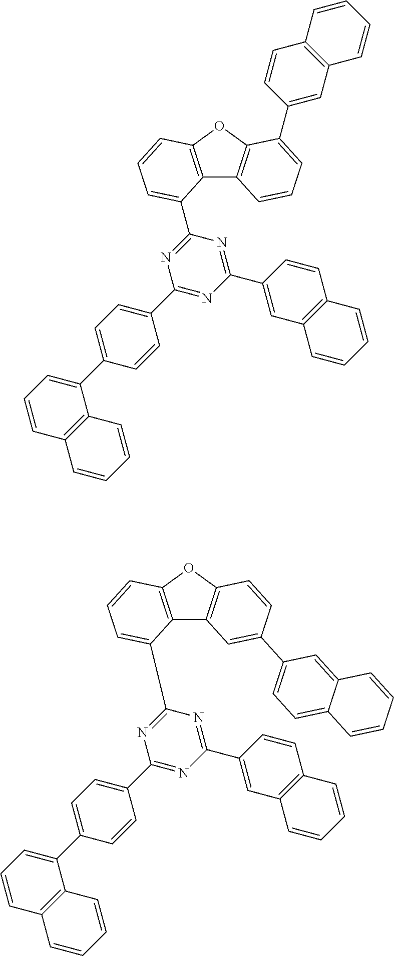 [SMILES: c1ccc2cc(-c3nc(-c4ccc(-c5cccc6ccccc56)cc4)nc(-c4cccc5oc6c(-c7ccc8ccccc8c7)cccc6c45)n3)ccc2c1 ; c1ccc2cc(-c3ccc4oc5cccc(-c6nc(-c7ccc(-c8cccc9ccccc89)cc7)nc(-c7ccc8ccccc8c7)n6)c5c4c3)ccc2c1]

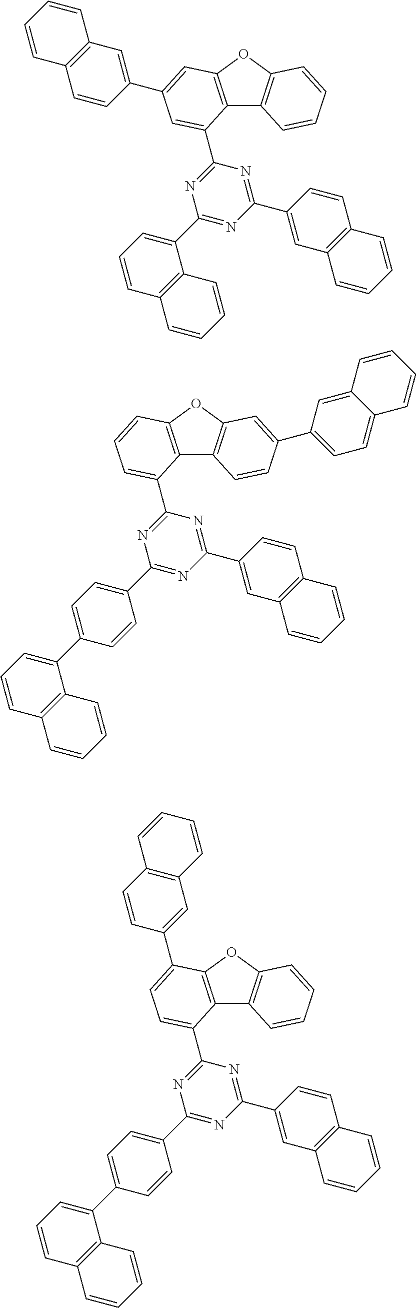 [SMILES: c1ccc2cc(-c3cc(-c4nc(-c5ccc6ccccc6c5)nc(-c5cccc6ccccc56)n4)c4c(c3)oc3ccccc34)ccc2c1 ; c1ccc2cc(-c3ccc4c(c3)oc3cccc(-c5nc(-c6ccc(-c7cccc8ccccc78)cc6)nc(-c6ccc7ccccc7c6)n5)c34)ccc2c1 ; c1ccc2cc(-c3nc(-c4ccc(-c5cccc6ccccc56)cc4)nc(-c4ccc(-c5ccc6ccccc6c5)c5oc6ccccc6c45)n3)ccc2c1]

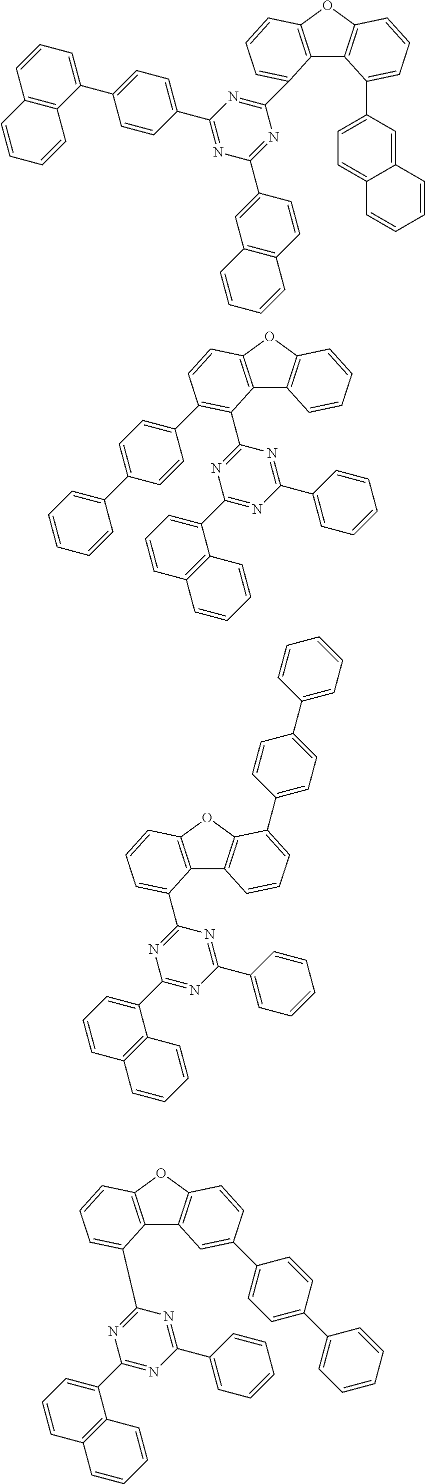

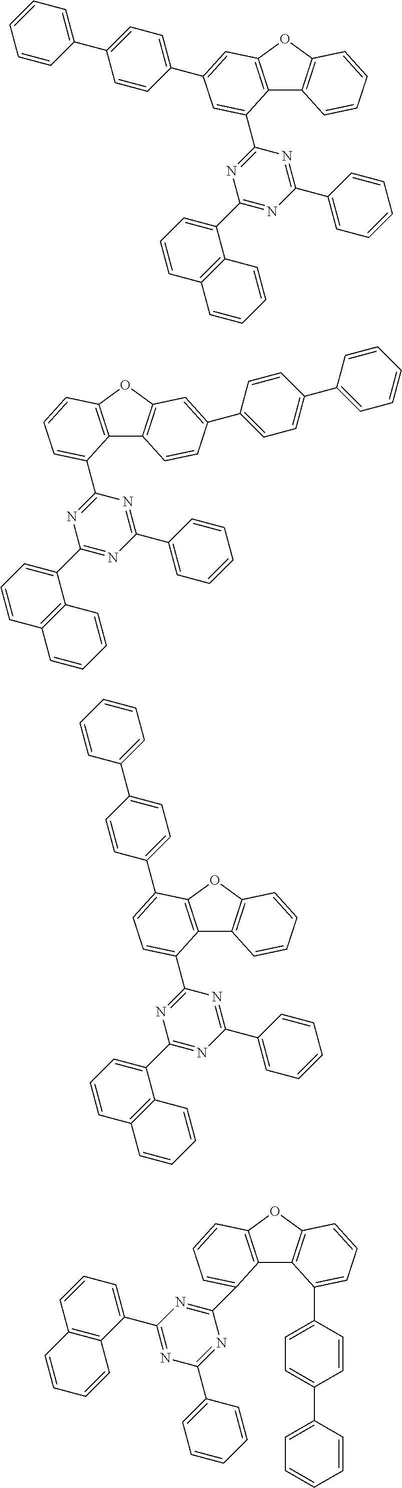

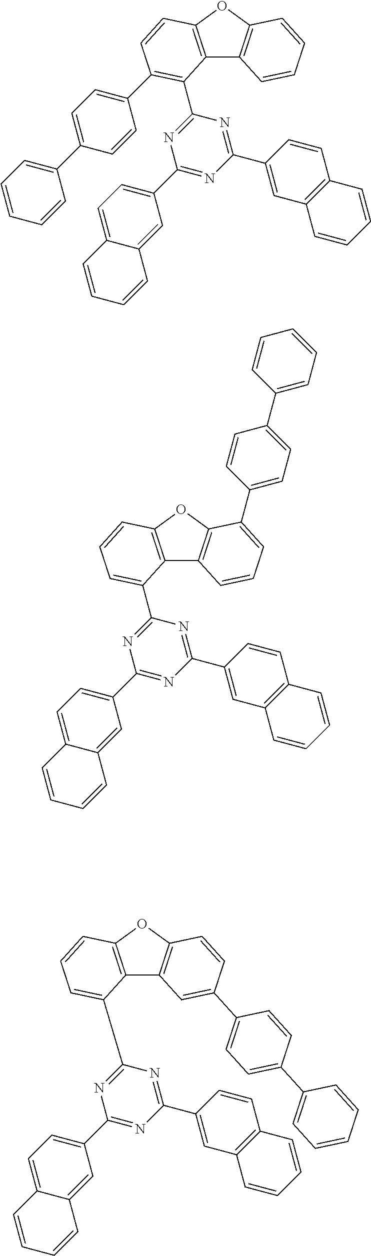 [SMILES: c1ccc(-c2ccc(-c3ccc4oc5ccccc5c4c3-c3nc(-c4ccc5ccccc5c4)nc(-c4ccc5ccccc5c4)n3)cc2)cc1 ; c1ccc(-c2ccc(-c3cccc4c3oc3cccc(-c5nc(-c6ccc7ccccc7c6)nc(-c6ccc7ccccc7c6)n5)c34)cc2)cc1 ; c1ccc(-c2ccc(-c3ccc4oc5cccc(-c6nc(-c7ccc8ccccc8c7)nc(-c7ccc8ccccc8c7)n6)c5c4c3)cc2)cc1]

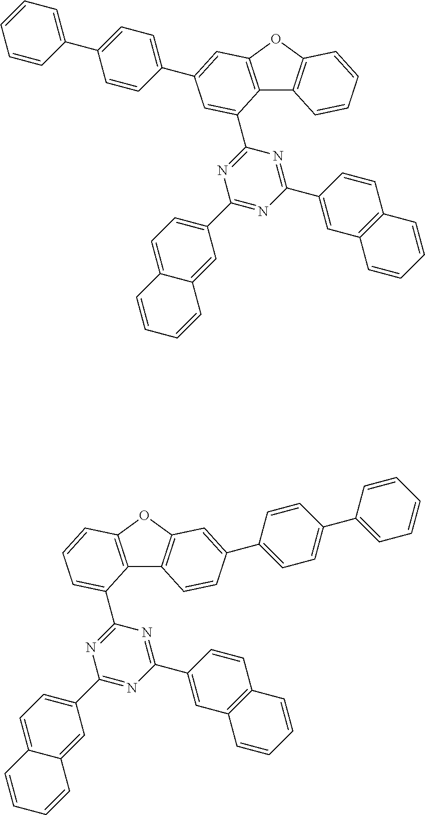 [SMILES: c1ccc(-c2ccc(-c3cc(-c4nc(-c5ccc6ccccc6c5)nc(-c5ccc6ccccc6c5)n4)c4c(c3)oc3ccccc34)cc2)cc1 ; c1ccc(-c2ccc(-c3ccc4c(c3)oc3cccc(-c5nc(-c6ccc7ccccc7c6)nc(-c6ccc7ccccc7c6)n5)c34)cc2)cc1]

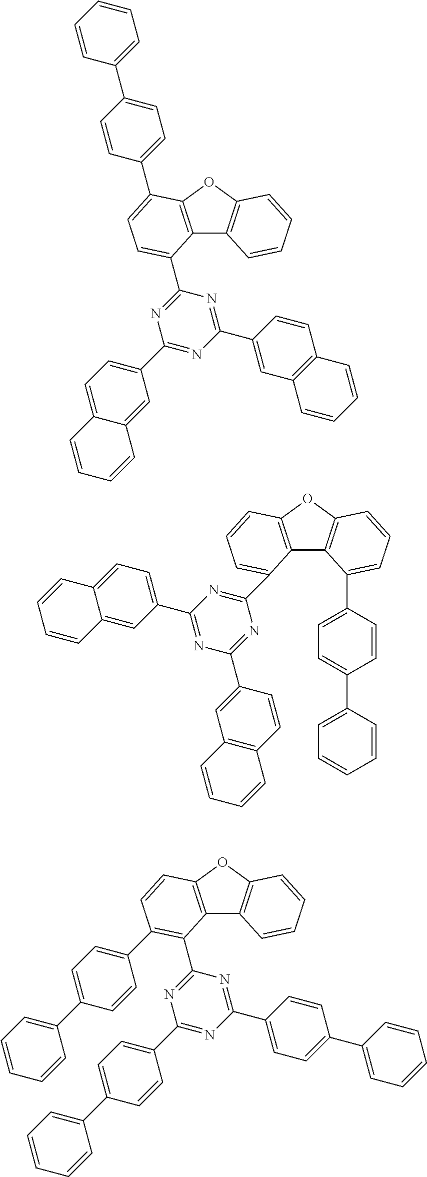 [SMILES: c1ccc(-c2ccc(-c3ccc(-c4nc(-c5ccc6ccccc6c5)nc(-c5ccc6ccccc6c5)n4)c4c3oc3ccccc34)cc2)cc1 ; c1ccc(-c2ccc(-c3cccc4oc5cccc(-c6nc(-c7ccc8ccccc8c7)nc(-c7ccc8ccccc8c7)n6)c5c34)cc2)cc1 ; c1ccc(-c2ccc(-c3nc(-c4ccc(-c5ccccc5)cc4)nc(-c4c(-c5ccc(-c6ccccc6)cc5)ccc5oc6ccccc6c45)n3)cc2)cc1]

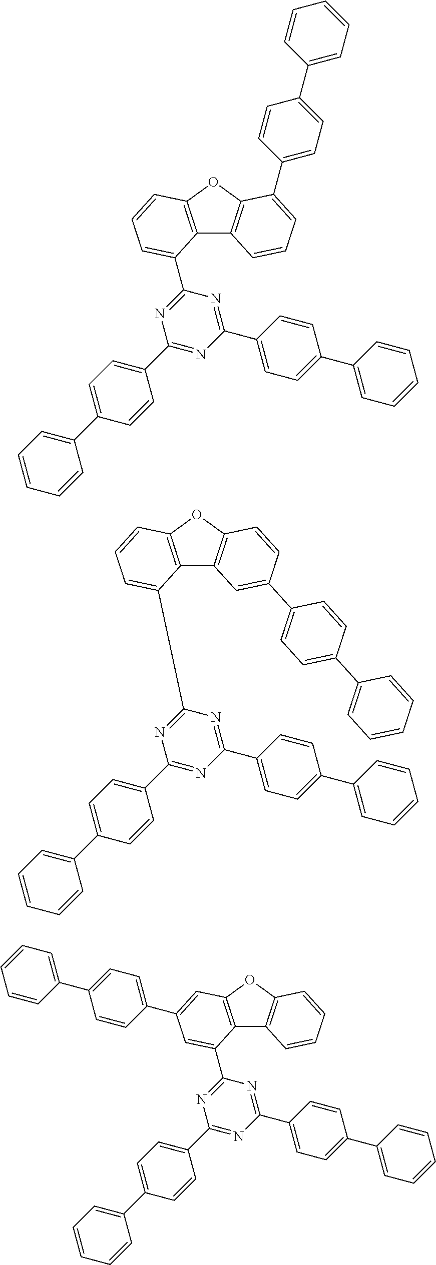

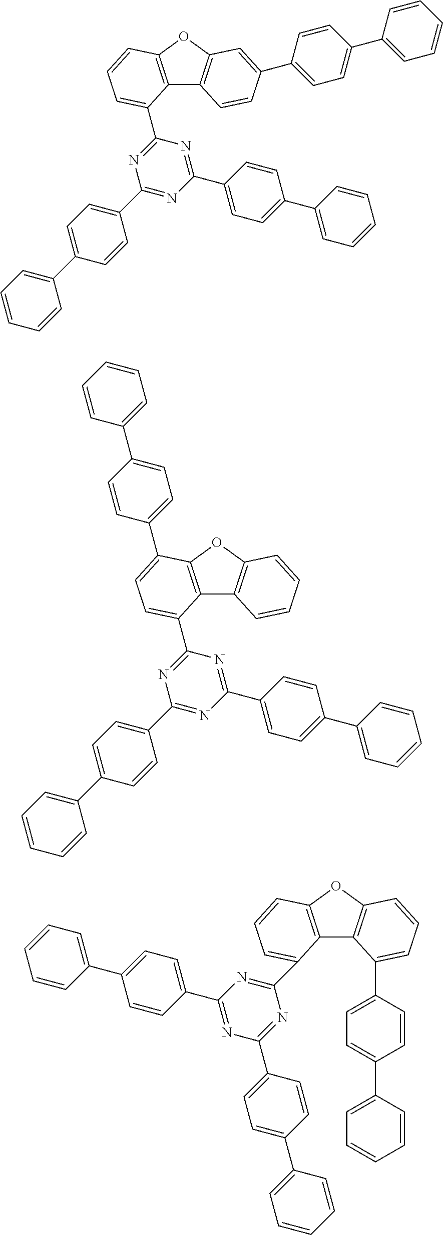 [SMILES: c1ccc(-c2ccc(-c3ccc4c(c3)oc3cccc(-c5nc(-c6ccc(-c7ccccc7)cc6)nc(-c6ccc(-c7ccccc7)cc6)n5)c34)cc2)cc1 ; c1ccc(-c2ccc(-c3nc(-c4ccc(-c5ccccc5)cc4)nc(-c4ccc(-c5ccc(-c6ccccc6)cc5)c5oc6ccccc6c45)n3)cc2)cc1 ; c1ccc(-c2ccc(-c3nc(-c4ccc(-c5ccccc5)cc4)nc(-c4cccc5oc6cccc(-c7ccc(-c8ccccc8)cc7)c6c45)n3)cc2)cc1]

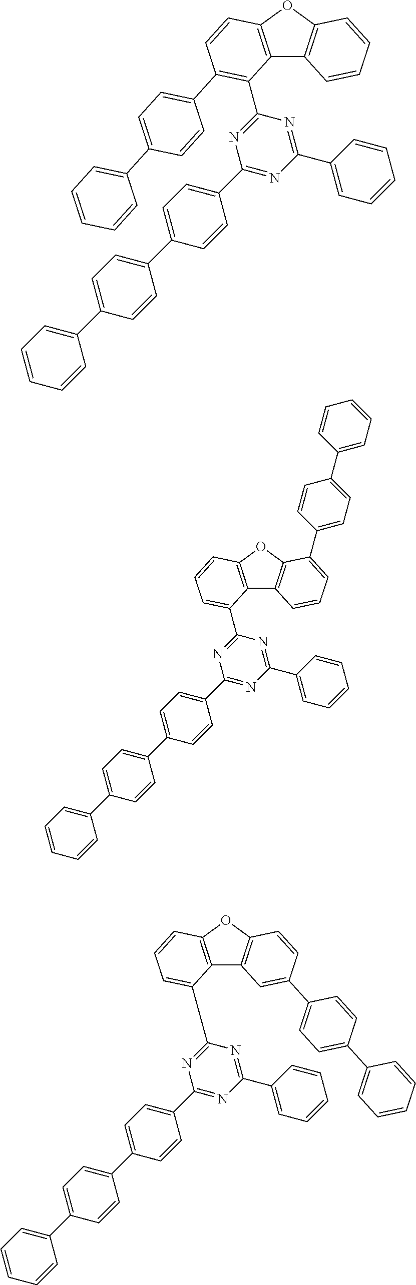 [SMILES: c1ccc(-c2ccc(-c3ccc(-c4nc(-c5ccccc5)nc(-c5c(-c6ccc(-c7ccccc7)cc6)ccc6oc7ccccc7c56)n4)cc3)cc2)cc1 ; c1ccc(-c2ccc(-c3ccc(-c4nc(-c5ccccc5)nc(-c5cccc6oc7c(-c8ccc(-c9ccccc9)cc8)cccc7c56)n4)cc3)cc2)cc1 ; c1ccc(-c2ccc(-c3ccc(-c4nc(-c5ccccc5)nc(-c5cccc6oc7ccc(-c8ccc(-c9ccccc9)cc8)cc7c56)n4)cc3)cc2)cc1]

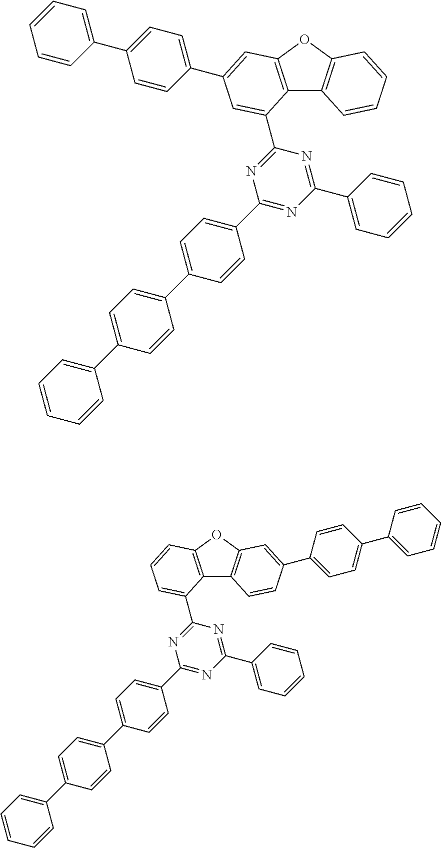 [SMILES: c1ccc(-c2ccc(-c3ccc(-c4nc(-c5ccccc5)nc(-c5cc(-c6ccc(-c7ccccc7)cc6)cc6oc7ccccc7c56)n4)cc3)cc2)cc1 ; c1ccc(-c2ccc(-c3ccc(-c4nc(-c5ccccc5)nc(-c5cccc6oc7cc(-c8ccc(-c9ccccc9)cc8)ccc7c56)n4)cc3)cc2)cc1]

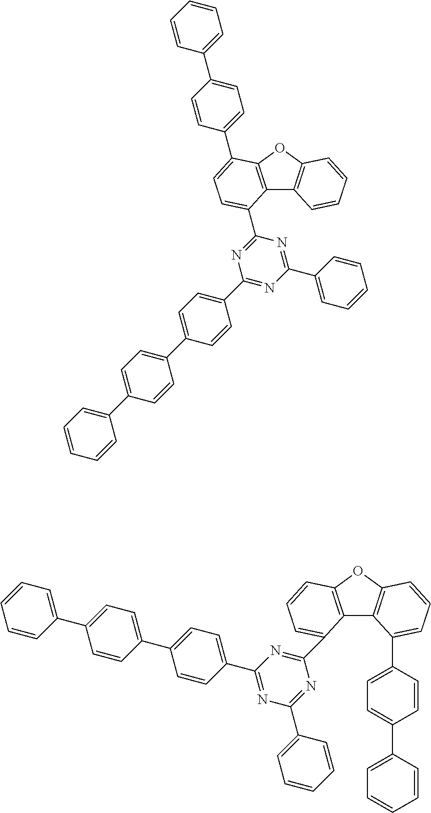 [SMILES: c1ccc(-c2ccc(-c3ccc(-c4nc(-c5ccccc5)nc(-c5ccc(-c6ccc(-c7ccccc7)cc6)c6oc7ccccc7c56)n4)cc3)cc2)cc1 ; c1ccc(-c2ccc(-c3ccc(-c4nc(-c5ccccc5)nc(-c5cccc6oc7cccc(-c8ccc(-c9ccccc9)cc8)c7c56)n4)cc3)cc2)cc1]

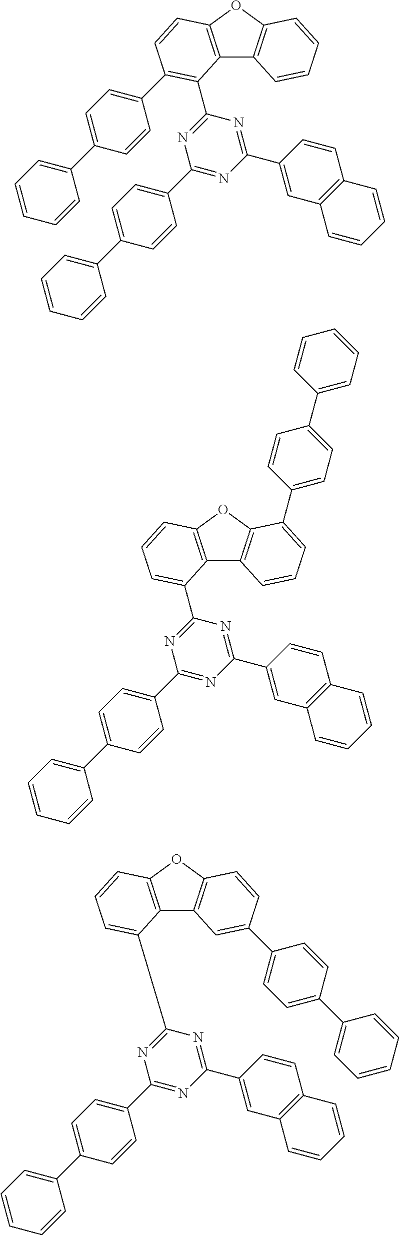 [SMILES: c1ccc(-c2ccc(-c3nc(-c4ccc5ccccc5c4)nc(-c4c(-c5ccc(-c6ccccc6)cc5)ccc5oc6ccccc6c45)n3)cc2)cc1 ; c1ccc(-c2ccc(-c3nc(-c4ccc5ccccc5c4)nc(-c4cccc5oc6c(-c7ccc(-c8ccccc8)cc7)cccc6c45)n3)cc2)cc1 ; c1ccc(-c2ccc(-c3ccc4oc5cccc(-c6nc(-c7ccc(-c8ccccc8)cc7)nc(-c7ccc8ccccc8c7)n6)c5c4c3)cc2)cc1]

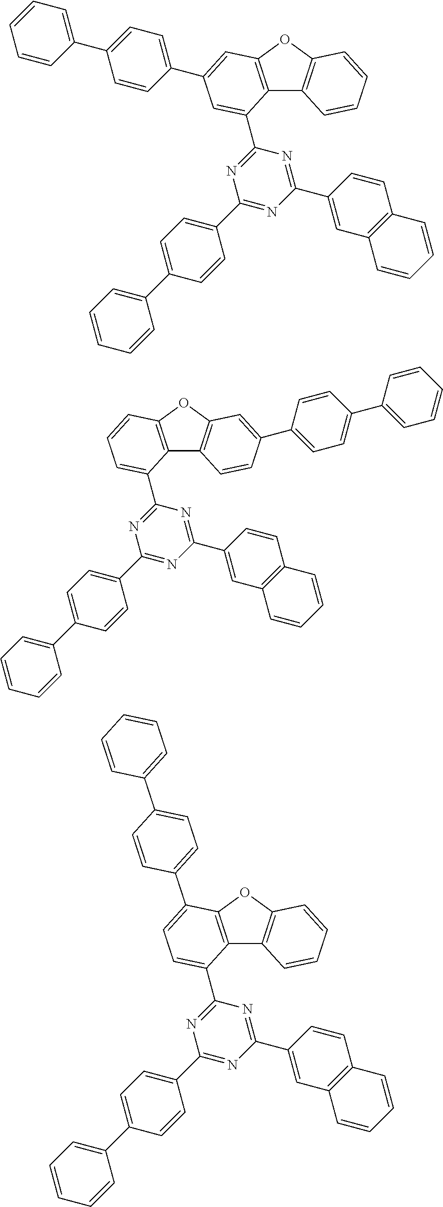 [SMILES: c1ccc(-c2ccc(-c3cc(-c4nc(-c5ccc(-c6ccccc6)cc5)nc(-c5ccc6ccccc6c5)n4)c4c(c3)oc3ccccc34)cc2)cc1 ; c1ccc(-c2ccc(-c3ccc4c(c3)oc3cccc(-c5nc(-c6ccc(-c7ccccc7)cc6)nc(-c6ccc7ccccc7c6)n5)c34)cc2)cc1 ; c1ccc(-c2ccc(-c3nc(-c4ccc5ccccc5c4)nc(-c4ccc(-c5ccc(-c6ccccc6)cc5)c5oc6ccccc6c45)n3)cc2)cc1]

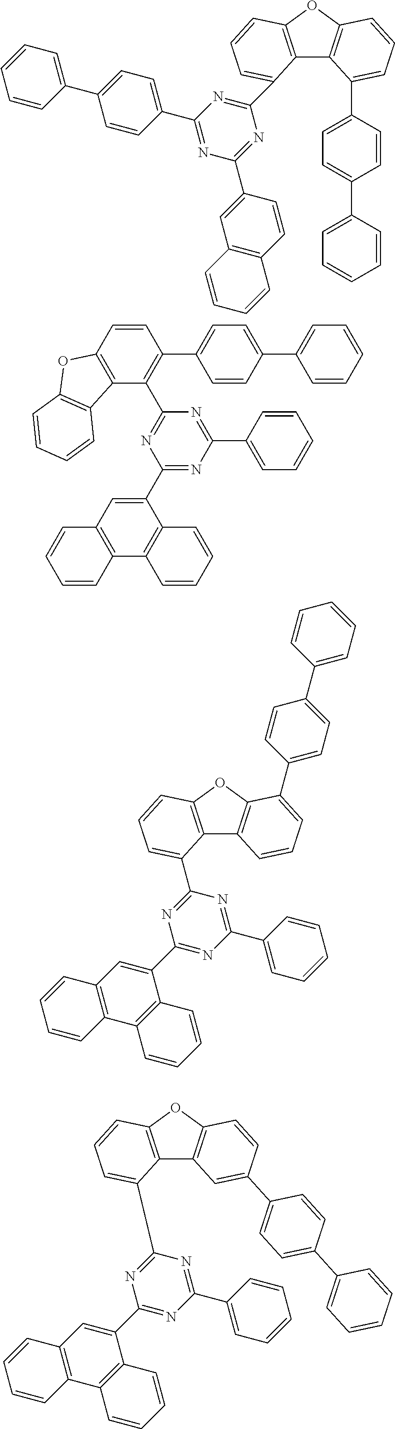

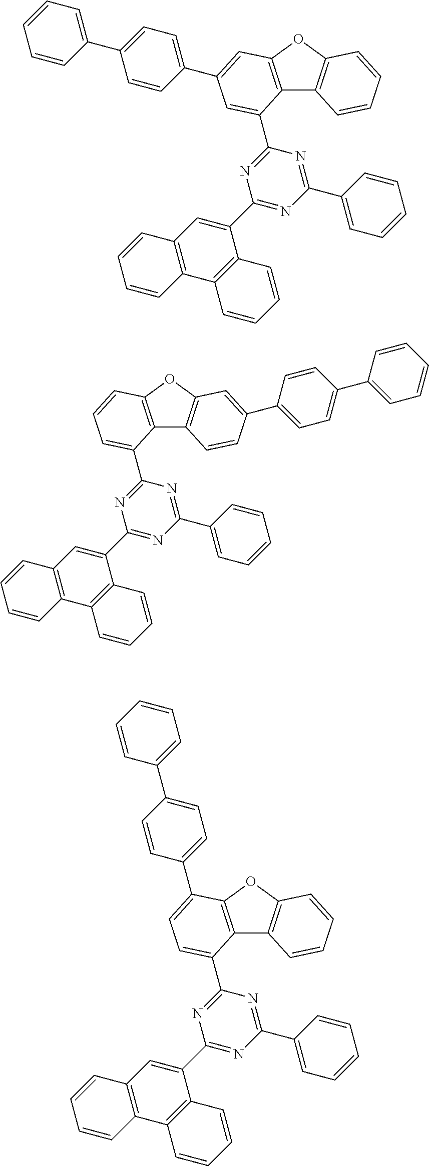 [SMILES: c1ccc(-c2ccc(-c3cc(-c4nc(-c5ccccc5)nc(-c5cc6ccccc6c6ccccc56)n4)c4c(c3)oc3ccccc34)cc2)cc1 ; c1ccc(-c2ccc(-c3ccc4c(c3)oc3cccc(-c5nc(-c6ccccc6)nc(-c6cc7ccccc7c7ccccc67)n5)c34)cc2)cc1 ; c1ccc(-c2ccc(-c3ccc(-c4nc(-c5ccccc5)nc(-c5cc6ccccc6c6ccccc56)n4)c4c3oc3ccccc34)cc2)cc1]

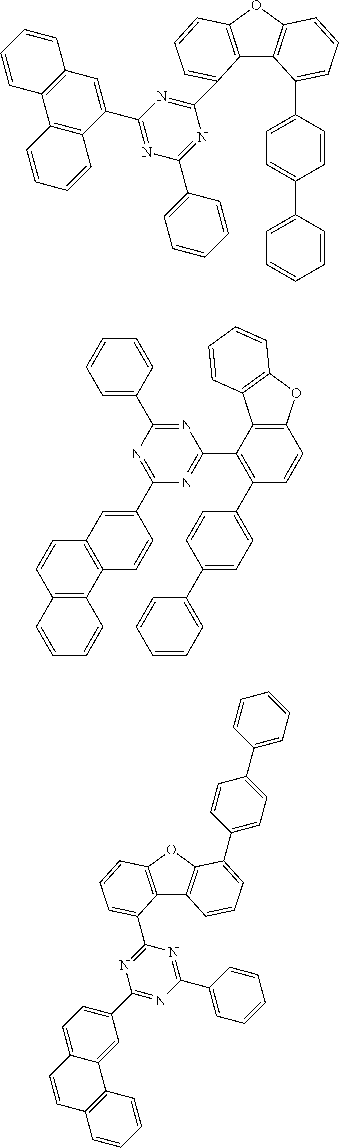 [SMILES: c1ccc(-c2ccc(-c3cccc4oc5cccc(-c6nc(-c7ccccc7)nc(-c7cc8ccccc8c8ccccc78)n6)c5c34)cc2)cc1 ; c1ccc(-c2ccc(-c3ccc4oc5ccccc5c4c3-c3nc(-c4ccccc4)nc(-c4ccc5c(ccc6ccccc65)c4)n3)cc2)cc1 ; c1ccc(-c2ccc(-c3cccc4c3oc3cccc(-c5nc(-c6ccccc6)nc(-c6ccc7ccc8ccccc8c7c6)n5)c34)cc2)cc1]

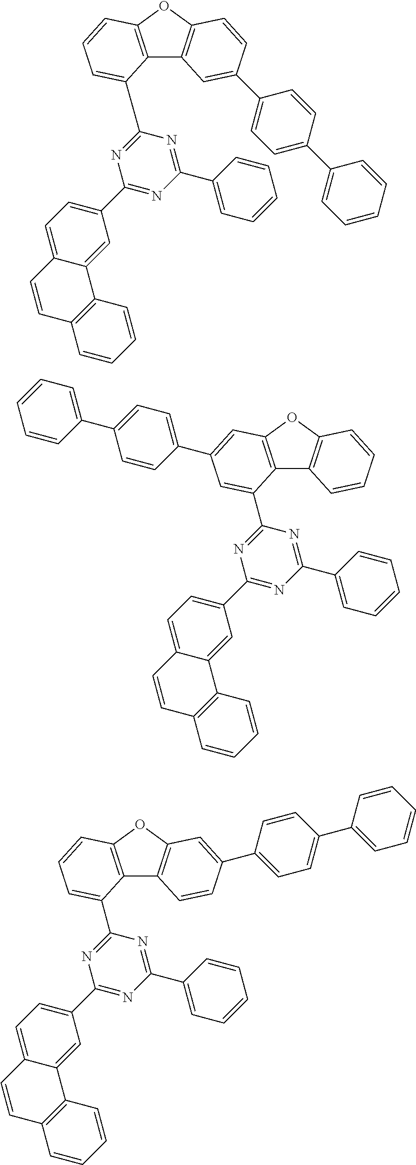

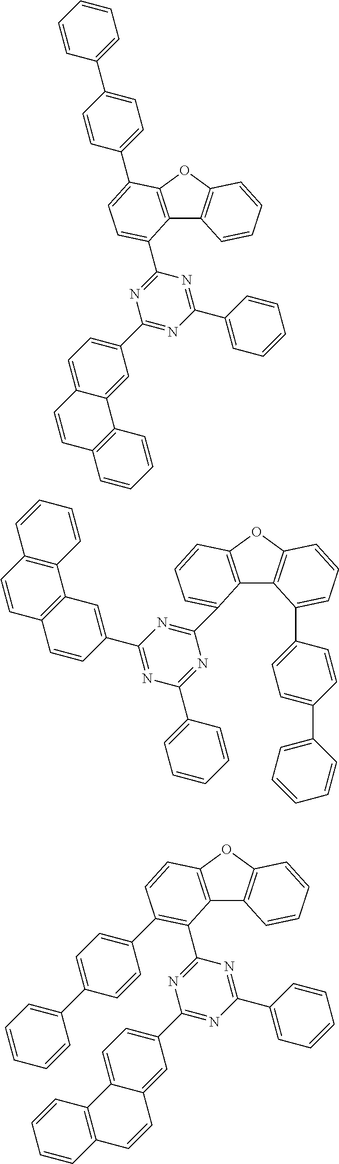

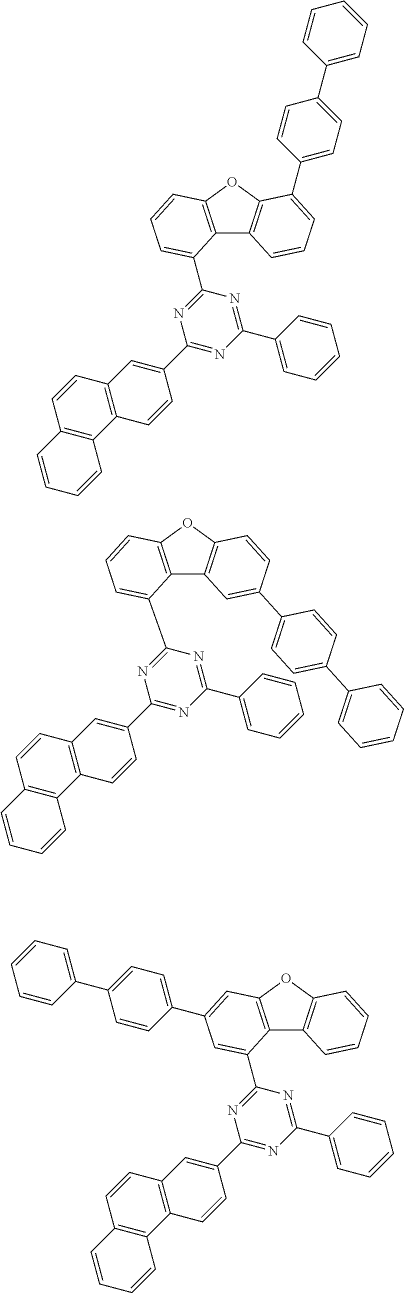 [SMILES: c1ccc(-c2ccc(-c3cccc4c3oc3cccc(-c5nc(-c6ccccc6)nc(-c6ccc7c(ccc8ccccc87)c6)n5)c34)cc2)cc1 ; c1ccc(-c2ccc(-c3ccc4oc5cccc(-c6nc(-c7ccccc7)nc(-c7ccc8c(ccc9ccccc98)c7)n6)c5c4c3)cc2)cc1 ; c1ccc(-c2ccc(-c3cc(-c4nc(-c5ccccc5)nc(-c5ccc6c(ccc7ccccc76)c5)n4)c4c(c3)oc3ccccc34)cc2)cc1]

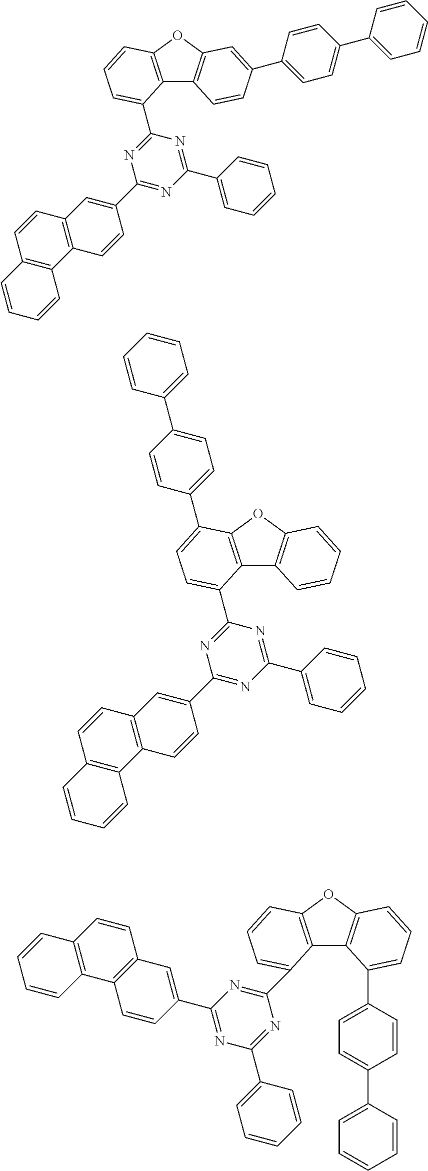 [SMILES: c1ccc(-c2ccc(-c3ccc4c(c3)oc3cccc(-c5nc(-c6ccccc6)nc(-c6ccc7c(ccc8ccccc87)c6)n5)c34)cc2)cc1 ; c1ccc(-c2ccc(-c3ccc(-c4nc(-c5ccccc5)nc(-c5ccc6c(ccc7ccccc76)c5)n4)c4c3oc3ccccc34)cc2)cc1 ; c1ccc(-c2ccc(-c3cccc4oc5cccc(-c6nc(-c7ccccc7)nc(-c7ccc8c(ccc9ccccc98)c7)n6)c5c34)cc2)cc1]

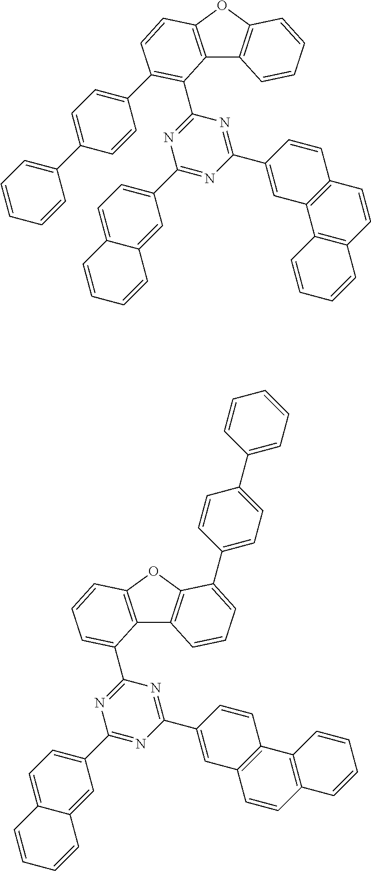 [SMILES: c1ccc(-c2ccc(-c3ccc4oc5ccccc5c4c3-c3nc(-c4ccc5ccccc5c4)nc(-c4ccc5ccc6ccccc6c5c4)n3)cc2)cc1 ; c1ccc(-c2ccc(-c3cccc4c3oc3cccc(-c5nc(-c6ccc7ccccc7c6)nc(-c6ccc7c(ccc8ccccc87)c6)n5)c34)cc2)cc1]

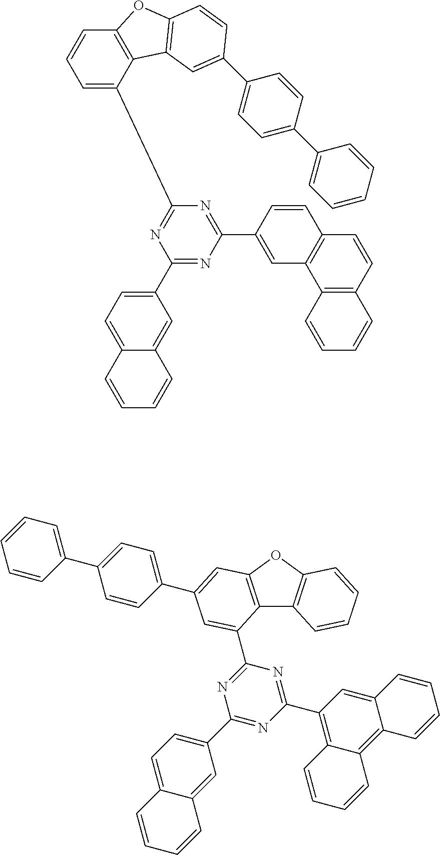 [SMILES: c1ccc(-c2ccc(-c3ccc4oc5cccc(-c6nc(-c7ccc8ccccc8c7)nc(-c7ccc8ccc9ccccc9c8c7)n6)c5c4c3)cc2)cc1 ; c1ccc(-c2ccc(-c3cc(-c4nc(-c5ccc6ccccc6c5)nc(-c5cc6ccccc6c6ccccc56)n4)c4c(c3)oc3ccccc34)cc2)cc1]

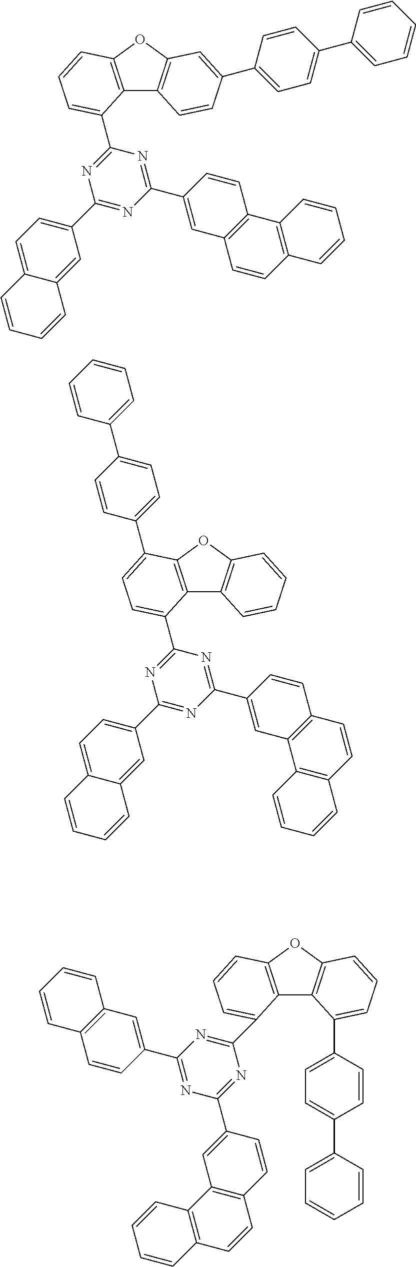 [SMILES: c1ccc(-c2ccc(-c3ccc4c(c3)oc3cccc(-c5nc(-c6ccc7ccccc7c6)nc(-c6ccc7c(ccc8ccccc87)c6)n5)c34)cc2)cc1 ; c1ccc(-c2ccc(-c3ccc(-c4nc(-c5ccc6ccccc6c5)nc(-c5ccc6ccc7ccccc7c6c5)n4)c4c3oc3ccccc34)cc2)cc1 ; c1ccc(-c2ccc(-c3cccc4oc5cccc(-c6nc(-c7ccc8ccccc8c7)nc(-c7ccc8ccc9ccccc9c8c7)n6)c5c34)cc2)cc1]

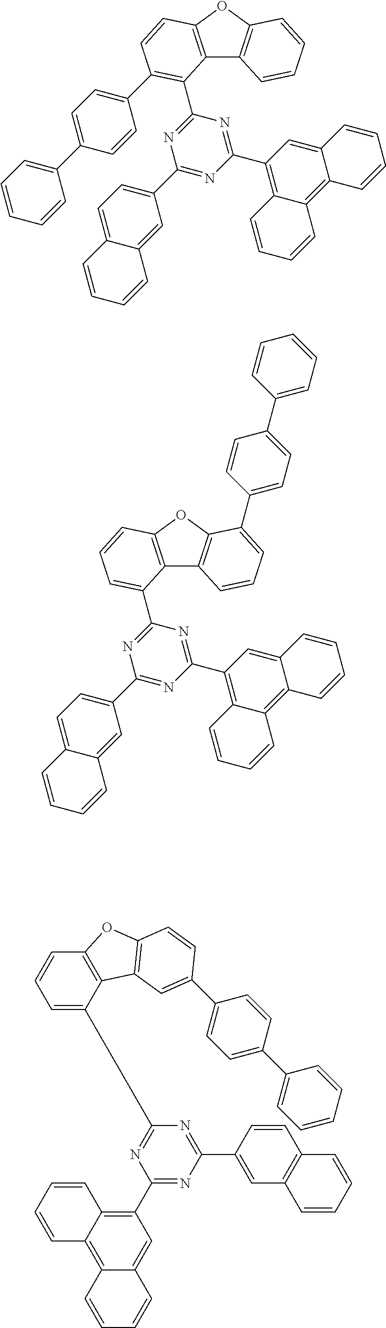 [SMILES: c1ccc(-c2ccc(-c3ccc4oc5ccccc5c4c3-c3nc(-c4ccc5ccccc5c4)nc(-c4cc5ccccc5c5ccccc45)n3)cc2)cc1 ; c1ccc(-c2ccc(-c3cccc4c3oc3cccc(-c5nc(-c6ccc7ccccc7c6)nc(-c6cc7ccccc7c7ccccc67)n5)c34)cc2)cc1 ; c1ccc(-c2ccc(-c3ccc4oc5cccc(-c6nc(-c7ccc8ccccc8c7)nc(-c7cc8ccccc8c8ccccc78)n6)c5c4c3)cc2)cc1]

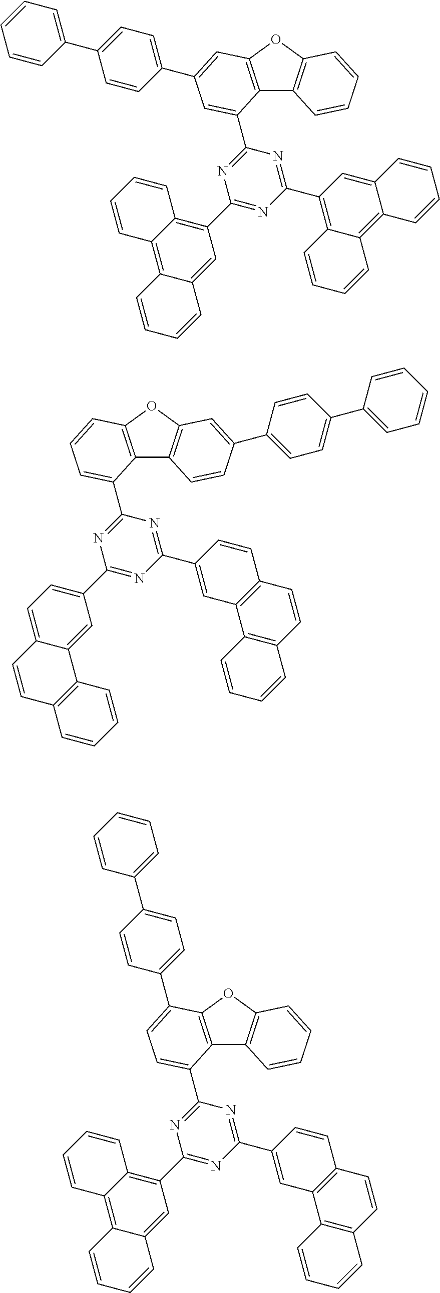 [SMILES: c1ccc(-c2ccc(-c3cc(-c4nc(-c5cc6ccccc6c6ccccc56)nc(-c5cc6ccccc6c6ccccc56)n4)c4c(c3)oc3ccccc34)cc2)cc1 ; c1ccc(-c2ccc(-c3ccc4c(c3)oc3cccc(-c5nc(-c6ccc7ccc8ccccc8c7c6)nc(-c6ccc7ccc8ccccc8c7c6)n5)c34)cc2)cc1 ; c1ccc(-c2ccc(-c3ccc(-c4nc(-c5ccc6ccc7ccccc7c6c5)nc(-c5cc6ccccc6c6ccccc56)n4)c4c3oc3ccccc34)cc2)cc1]

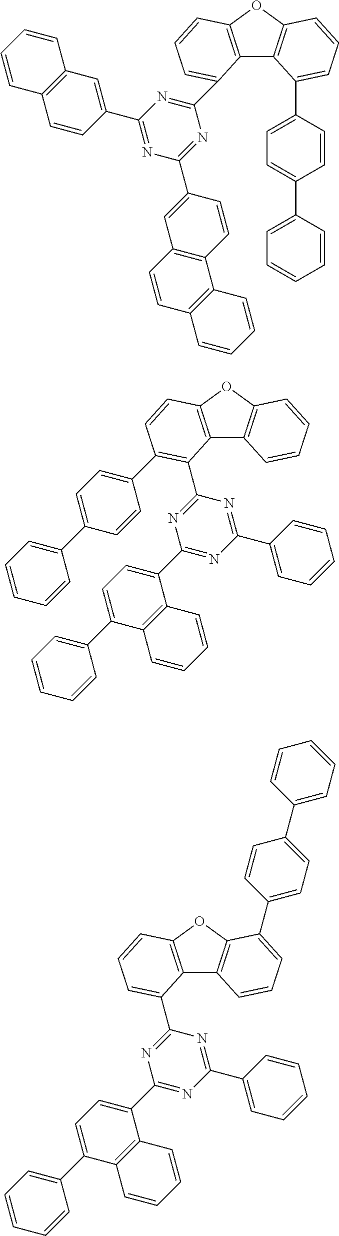

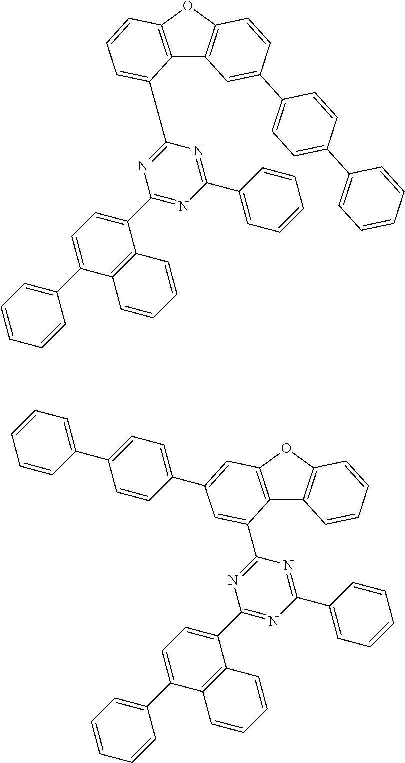 [SMILES: c1ccc(-c2ccc(-c3ccc4oc5cccc(-c6nc(-c7ccccc7)nc(-c7ccc(-c8ccccc8)c8ccccc78)n6)c5c4c3)cc2)cc1 ; c1ccc(-c2ccc(-c3cc(-c4nc(-c5ccccc5)nc(-c5ccc(-c6ccccc6)c6ccccc56)n4)c4c(c3)oc3ccccc34)cc2)cc1]

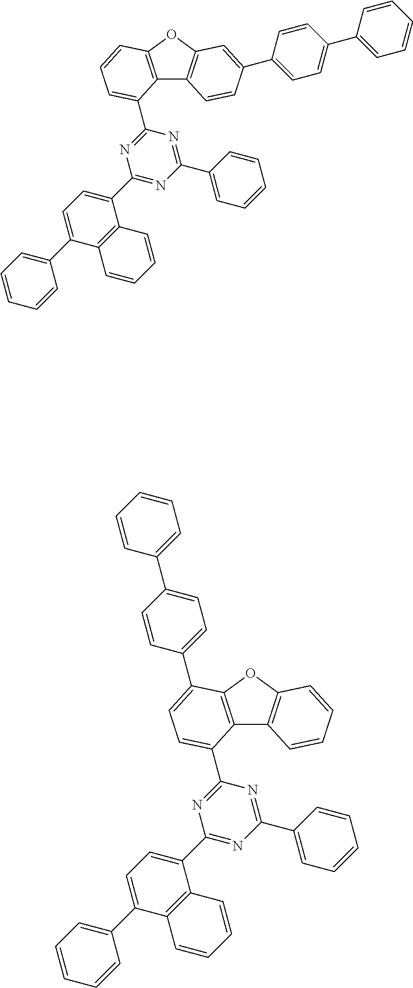 [SMILES: c1ccc(-c2ccc(-c3ccc4c(c3)oc3cccc(-c5nc(-c6ccccc6)nc(-c6ccc(-c7ccccc7)c7ccccc67)n5)c34)cc2)cc1 ; c1ccc(-c2ccc(-c3ccc(-c4nc(-c5ccccc5)nc(-c5ccc(-c6ccccc6)c6ccccc56)n4)c4c3oc3ccccc34)cc2)cc1]

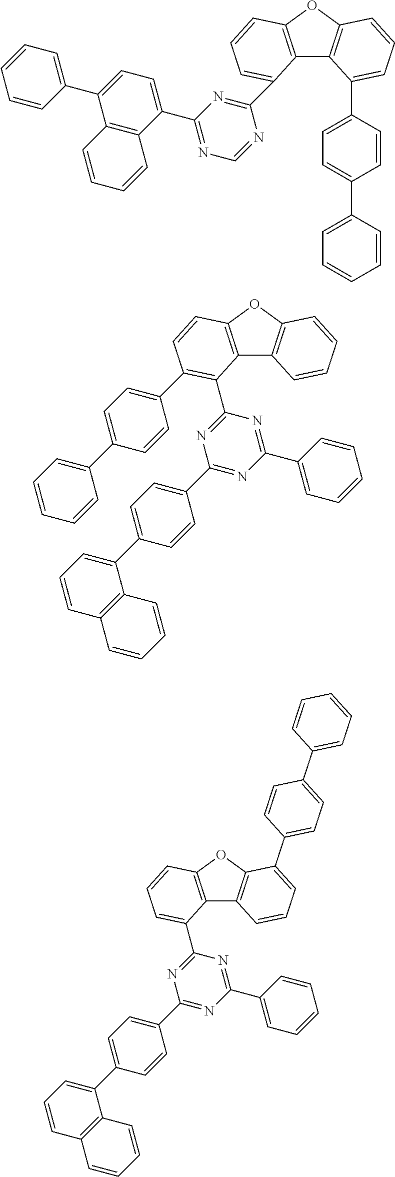 [SMILES: c1ccc(-c2ccc(-c3cccc4oc5cccc(-c6ncnc(-c7ccc(-c8ccccc8)c8ccccc78)n6)c5c34)cc2)cc1 ; c1ccc(-c2ccc(-c3ccc4oc5ccccc5c4c3-c3nc(-c4ccccc4)nc(-c4ccc(-c5cccc6ccccc56)cc4)n3)cc2)cc1 ; c1ccc(-c2ccc(-c3cccc4c3oc3cccc(-c5nc(-c6ccccc6)nc(-c6ccc(-c7cccc8ccccc78)cc6)n5)c34)cc2)cc1]

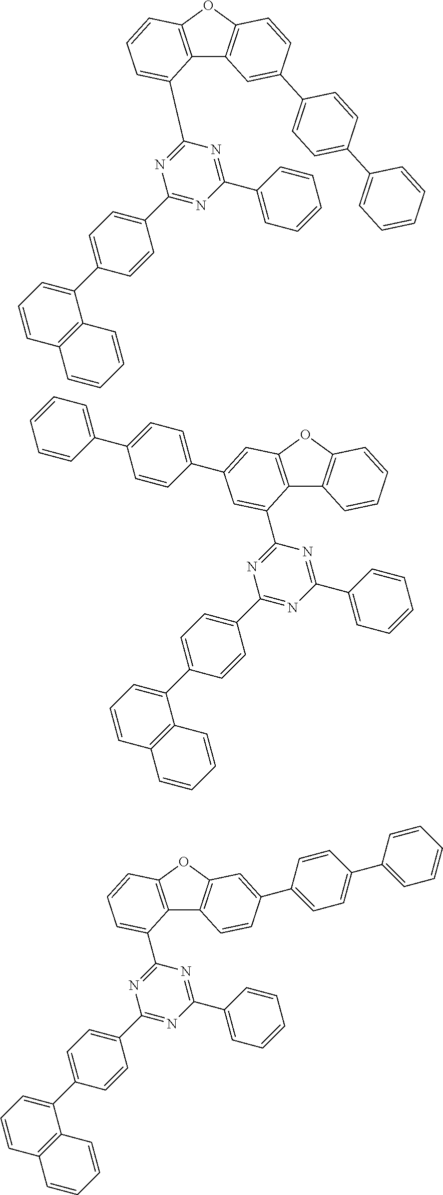

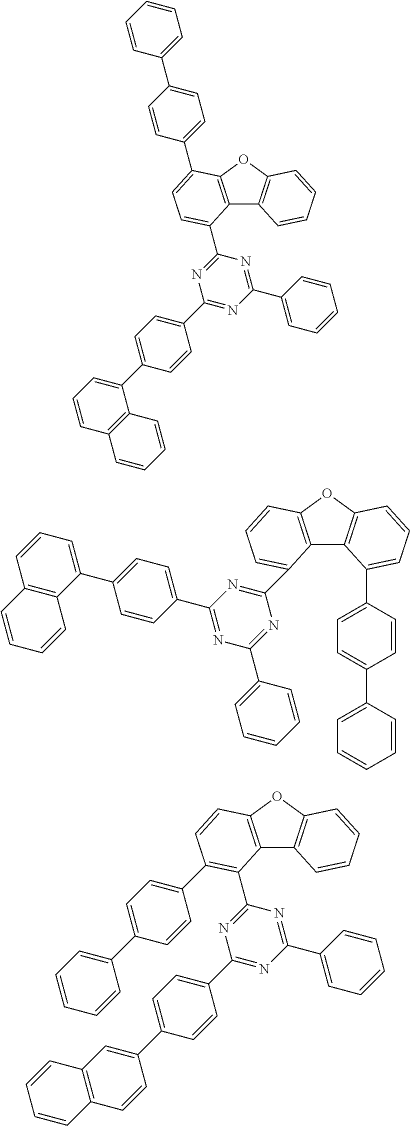 [SMILES: c1ccc(-c2ccc(-c3ccc(-c4nc(-c5ccccc5)nc(-c5ccc(-c6cccc7ccccc67)cc5)n4)c4c3oc3ccccc34)cc2)cc1 ; c1ccc(-c2ccc(-c3cccc4oc5cccc(-c6nc(-c7ccccc7)nc(-c7ccc(-c8cccc9ccccc89)cc7)n6)c5c34)cc2)cc1 ; c1ccc(-c2ccc(-c3ccc4oc5ccccc5c4c3-c3nc(-c4ccccc4)nc(-c4ccc(-c5ccc6ccccc6c5)cc4)n3)cc2)cc1]

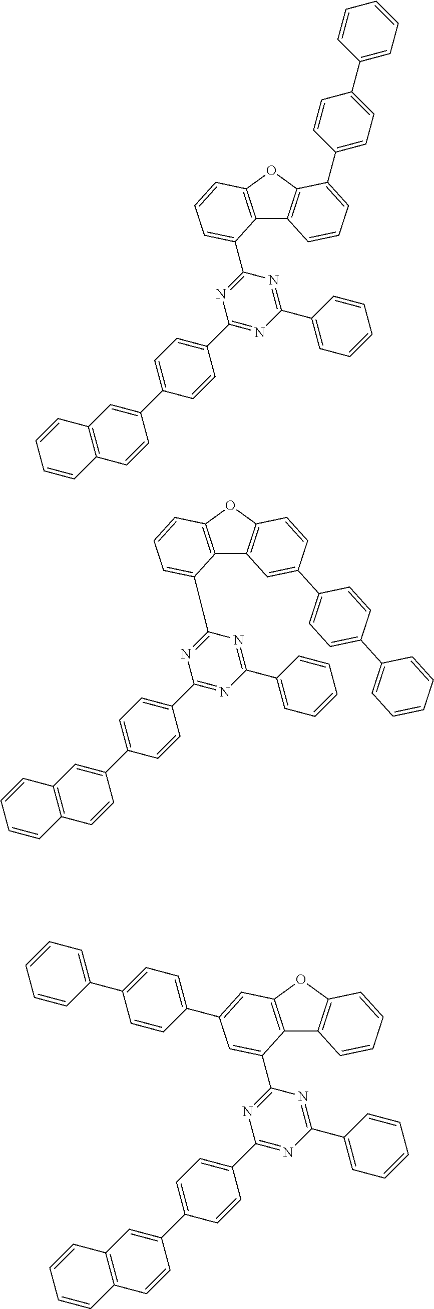 [SMILES: c1ccc(-c2ccc(-c3cccc4c3oc3cccc(-c5nc(-c6ccccc6)nc(-c6ccc(-c7ccc8ccccc8c7)cc6)n5)c34)cc2)cc1 ; c1ccc(-c2ccc(-c3ccc4oc5cccc(-c6nc(-c7ccccc7)nc(-c7ccc(-c8ccc9ccccc9c8)cc7)n6)c5c4c3)cc2)cc1 ; c1ccc(-c2ccc(-c3cc(-c4nc(-c5ccccc5)nc(-c5ccc(-c6ccc7ccccc7c6)cc5)n4)c4c(c3)oc3ccccc34)cc2)cc1]

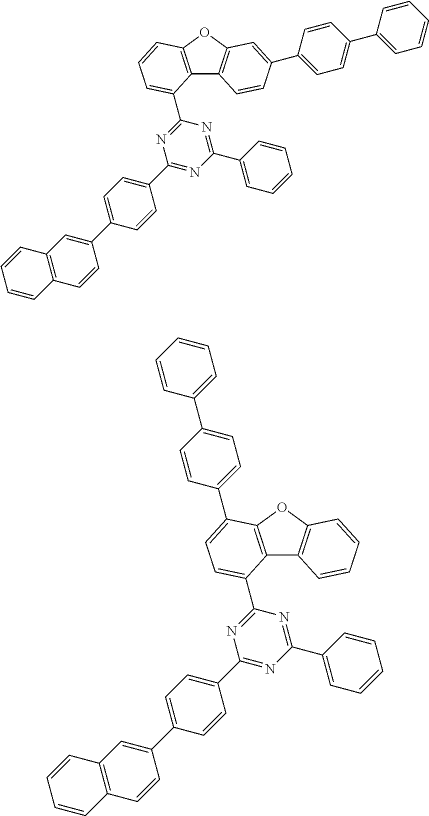 [SMILES: c1ccc(-c2ccc(-c3ccc4c(c3)oc3cccc(-c5nc(-c6ccccc6)nc(-c6ccc(-c7ccc8ccccc8c7)cc6)n5)c34)cc2)cc1 ; c1ccc(-c2ccc(-c3ccc(-c4nc(-c5ccccc5)nc(-c5ccc(-c6ccc7ccccc7c6)cc5)n4)c4c3oc3ccccc34)cc2)cc1]

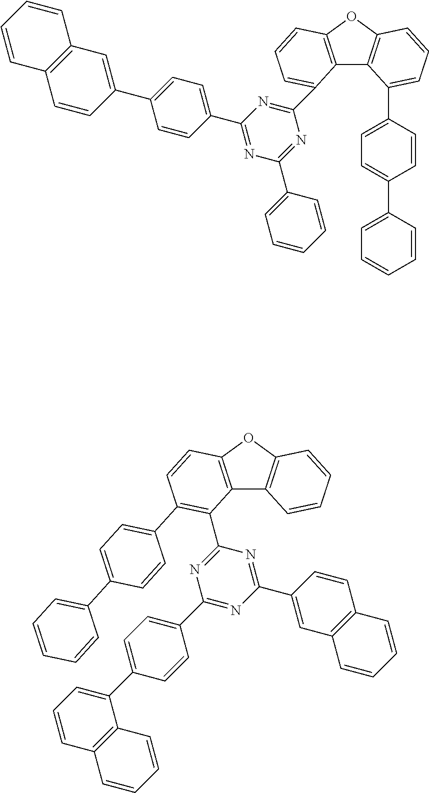 [SMILES: c1ccc(-c2ccc(-c3cccc4oc5cccc(-c6nc(-c7ccccc7)nc(-c7ccc(-c8ccc9ccccc9c8)cc7)n6)c5c34)cc2)cc1 ; c1ccc(-c2ccc(-c3ccc4oc5ccccc5c4c3-c3nc(-c4ccc(-c5cccc6ccccc56)cc4)nc(-c4ccc5ccccc5c4)n3)cc2)cc1]

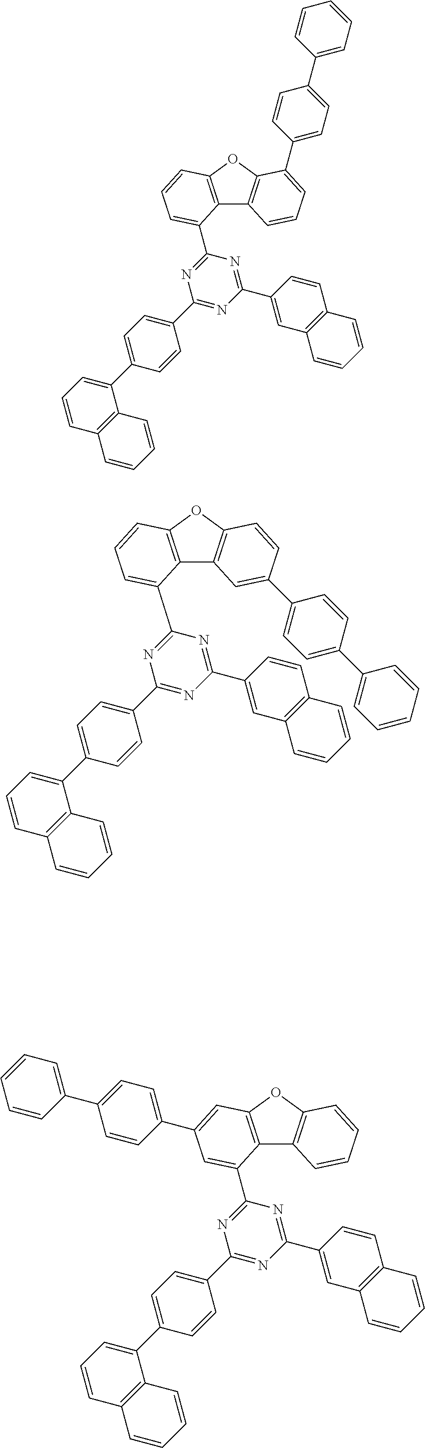 [SMILES: c1ccc(-c2ccc(-c3cccc4c3oc3cccc(-c5nc(-c6ccc(-c7cccc8ccccc78)cc6)nc(-c6ccc7ccccc7c6)n5)c34)cc2)cc1 ; c1ccc(-c2ccc(-c3ccc4oc5cccc(-c6nc(-c7ccc(-c8cccc9ccccc89)cc7)nc(-c7ccc8ccccc8c7)n6)c5c4c3)cc2)cc1 ; c1ccc(-c2ccc(-c3cc(-c4nc(-c5ccc(-c6cccc7ccccc67)cc5)nc(-c5ccc6ccccc6c5)n4)c4c(c3)oc3ccccc34)cc2)cc1]

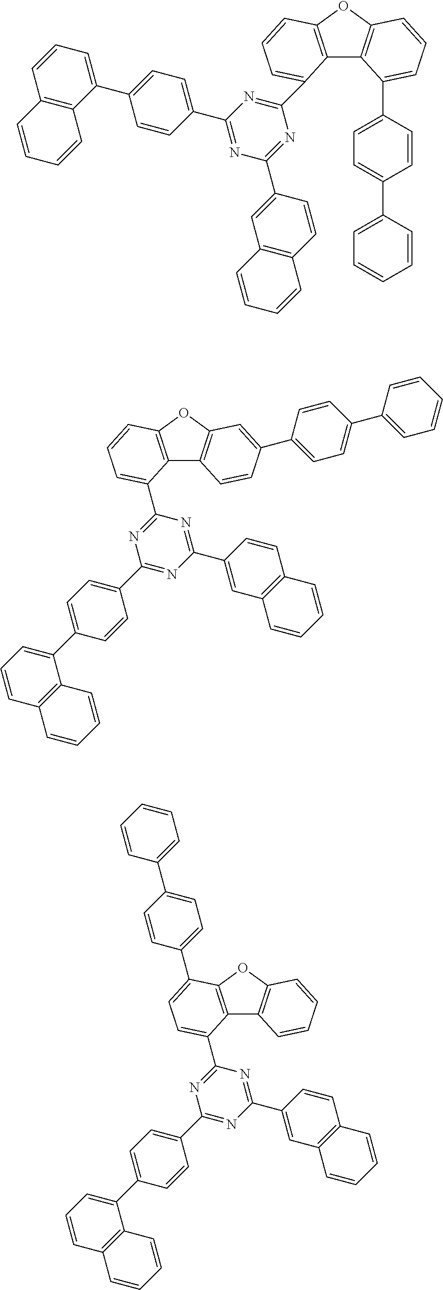 [SMILES: c1ccc(-c2ccc(-c3cccc4oc5cccc(-c6nc(-c7ccc(-c8cccc9ccccc89)cc7)nc(-c7ccc8ccccc8c7)n6)c5c34)cc2)cc1 ; c1ccc(-c2ccc(-c3ccc4c(c3)oc3cccc(-c5nc(-c6ccc(-c7cccc8ccccc78)cc6)nc(-c6ccc7ccccc7c6)n5)c34)cc2)cc1 ; c1ccc(-c2ccc(-c3ccc(-c4nc(-c5ccc(-c6cccc7ccccc67)cc5)nc(-c5ccc6ccccc6c5)n4)c4c3oc3ccccc34)cc2)cc1]

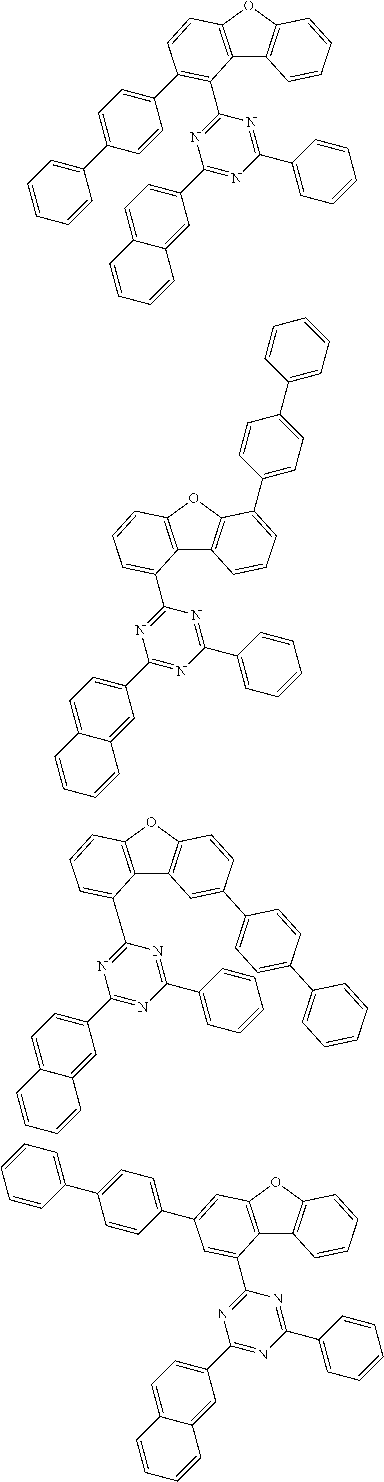

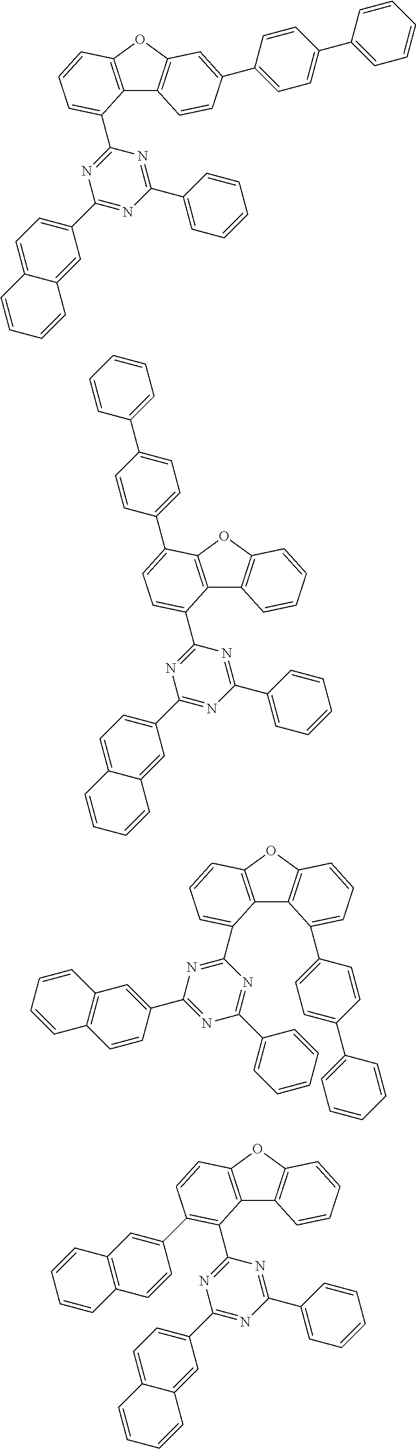 [SMILES: c1ccc(-c2ccc(-c3ccc4c(c3)oc3cccc(-c5nc(-c6ccccc6)nc(-c6ccc7ccccc7c6)n5)c34)cc2)cc1 ; c1ccc(-c2ccc(-c3ccc(-c4nc(-c5ccccc5)nc(-c5ccc6ccccc6c5)n4)c4c3oc3ccccc34)cc2)cc1 ; c1ccc(-c2ccc(-c3cccc4oc5cccc(-c6nc(-c7ccccc7)nc(-c7ccc8ccccc8c7)n6)c5c34)cc2)cc1 ; c1ccc(-c2nc(-c3ccc4ccccc4c3)nc(-c3c(-c4ccc5ccccc5c4)ccc4oc5ccccc5c34)n2)cc1]

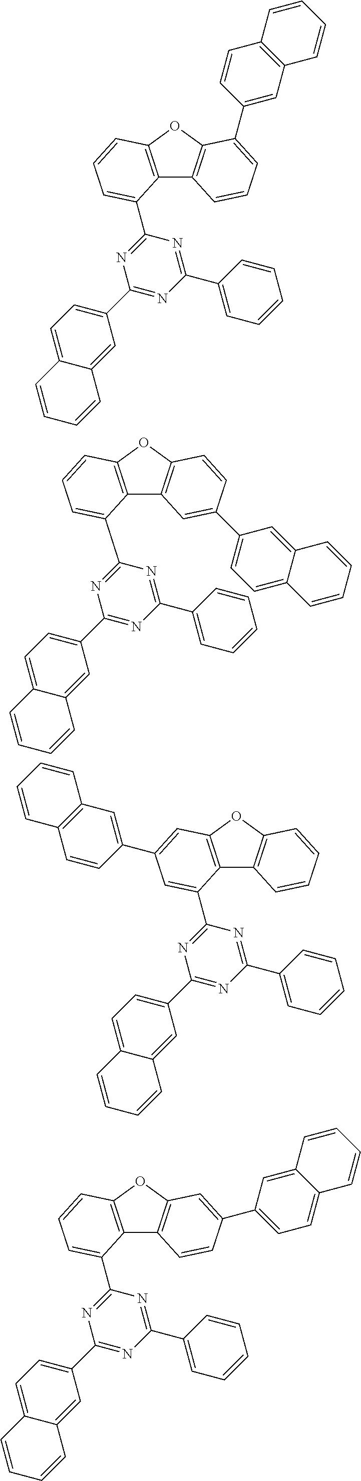

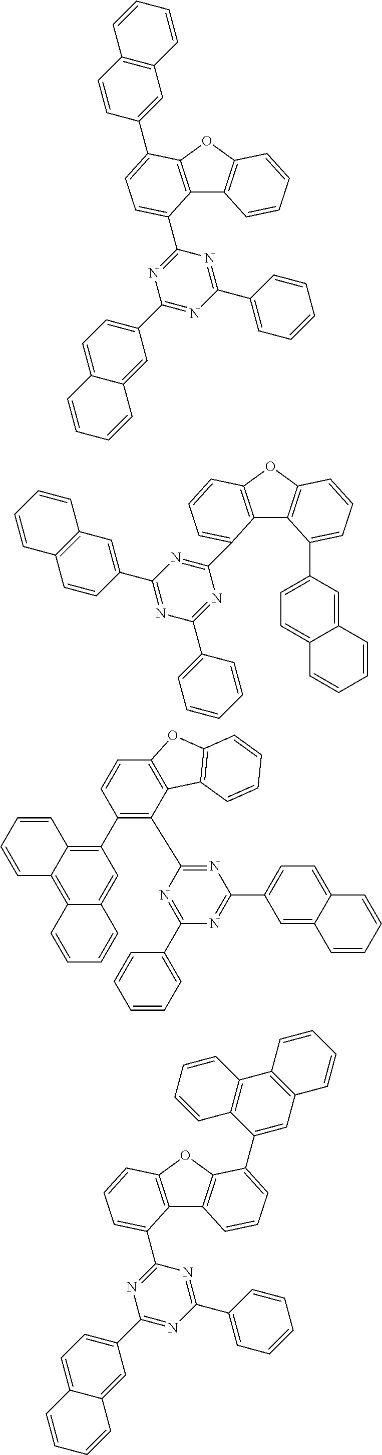

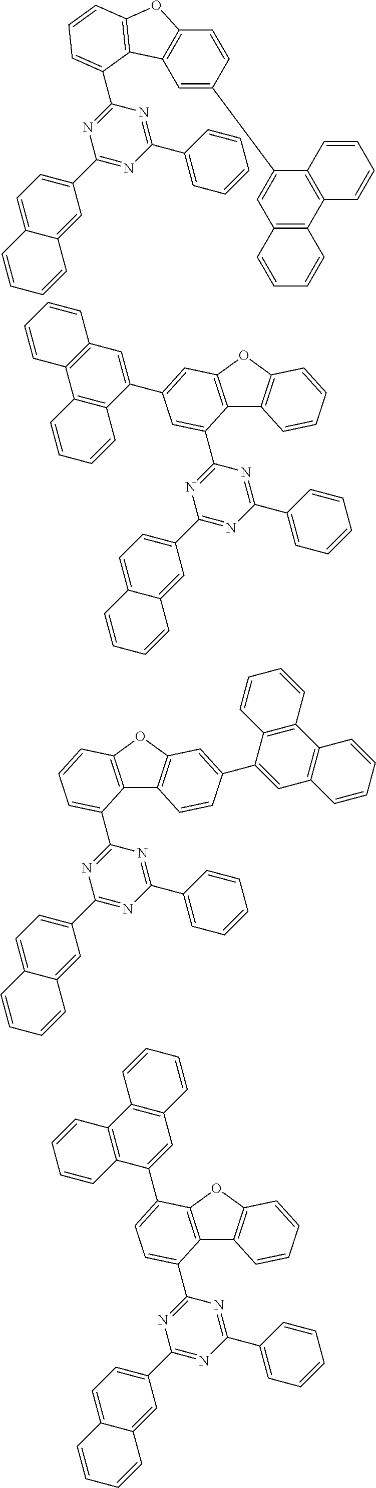

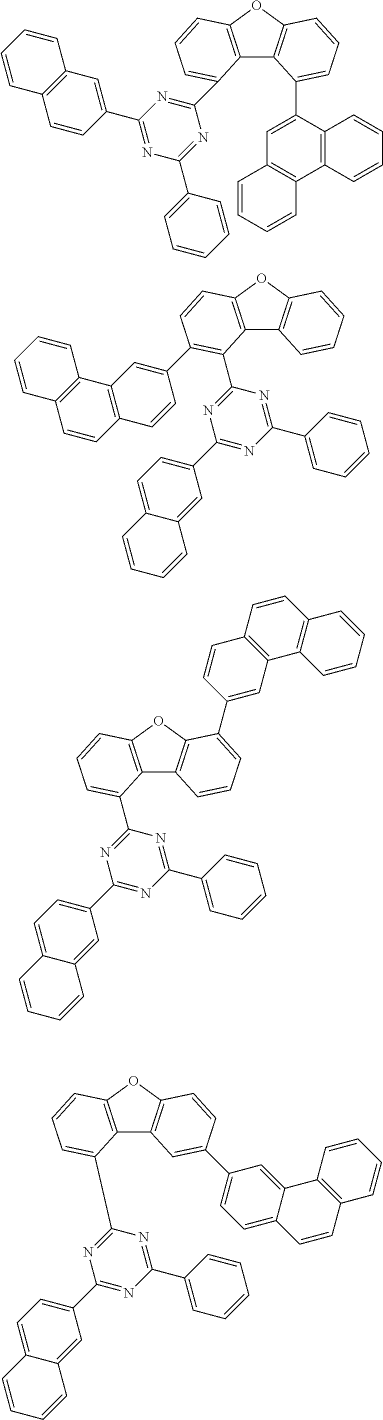

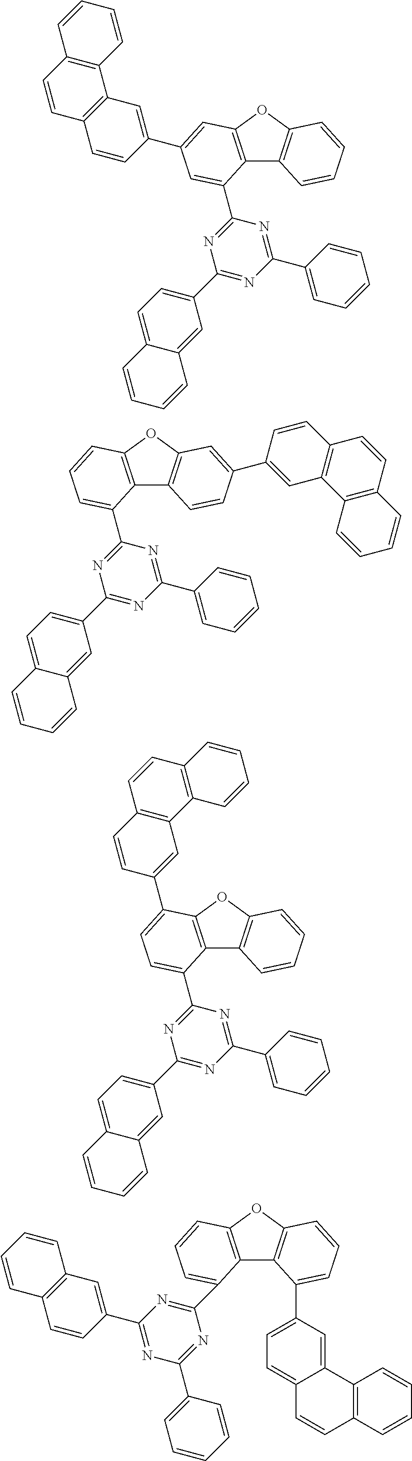

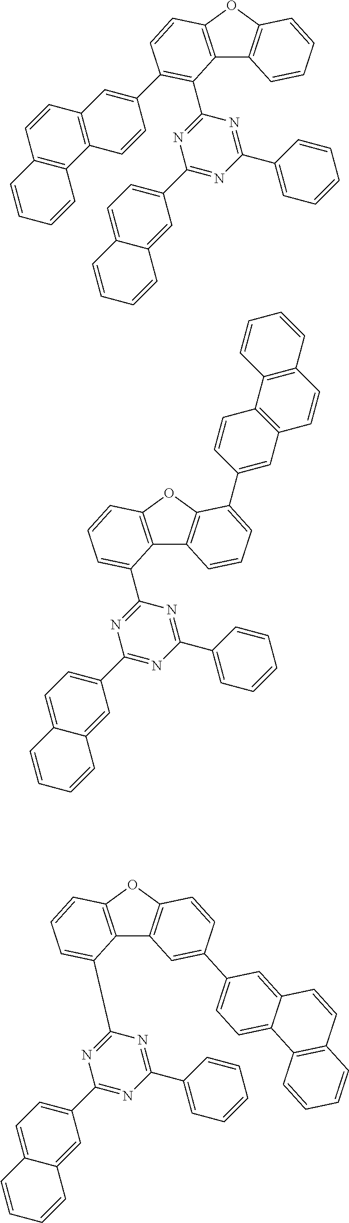 [SMILES: c1ccc(-c2nc(-c3ccc4ccccc4c3)nc(-c3c(-c4ccc5c(ccc6ccccc65)c4)ccc4oc5ccccc5c34)n2)cc1 ; c1ccc(-c2nc(-c3ccc4ccccc4c3)nc(-c3cccc4oc5c(-c6ccc7c(ccc8ccccc87)c6)cccc5c34)n2)cc1 ; c1ccc(-c2nc(-c3ccc4ccccc4c3)nc(-c3cccc4oc5ccc(-c6ccc7c(ccc8ccccc87)c6)cc5c34)n2)cc1]

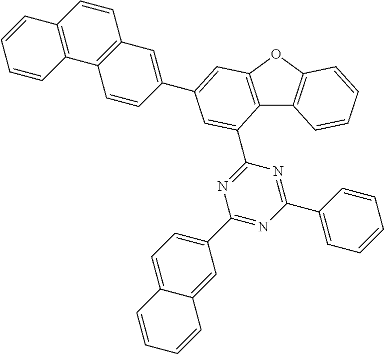 [SMILES: c1ccc(-c2nc(-c3ccc4ccccc4c3)nc(-c3cc(-c4ccc5c(ccc6ccccc65)c4)cc4oc5ccccc5c34)n2)cc1]

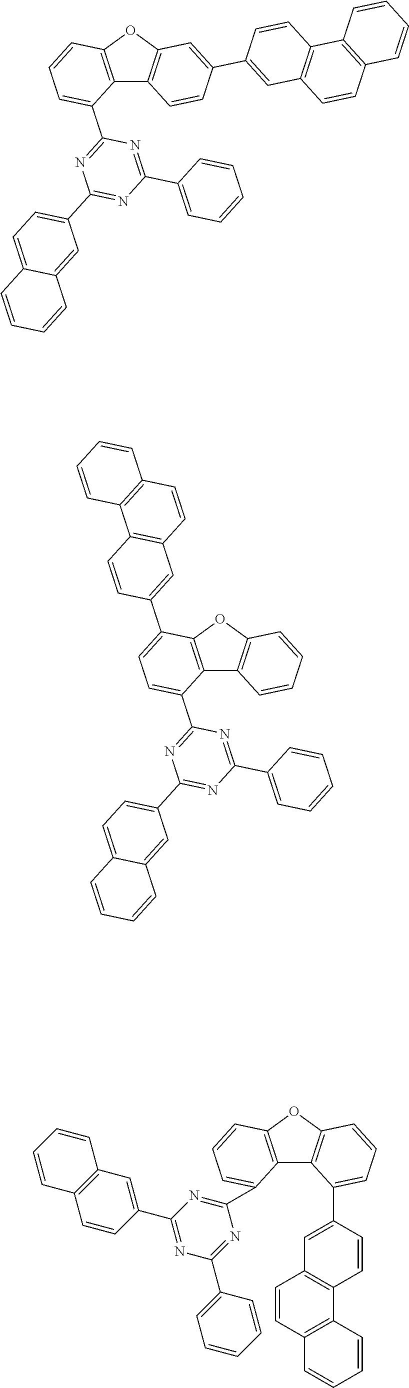 [SMILES: c1ccc(-c2nc(-c3ccc4ccccc4c3)nc(-c3cccc4oc5cc(-c6ccc7c(ccc8ccccc87)c6)ccc5c34)n2)cc1 ; c1ccc(-c2nc(-c3ccc4ccccc4c3)nc(-c3ccc(-c4ccc5c(ccc6ccccc65)c4)c4oc5ccccc5c34)n2)cc1 ; c1ccc(-c2nc(-c3ccc4ccccc4c3)nc(-c3cccc4oc5cccc(-c6ccc7c(ccc8ccccc87)c6)c5c34)n2)cc1]

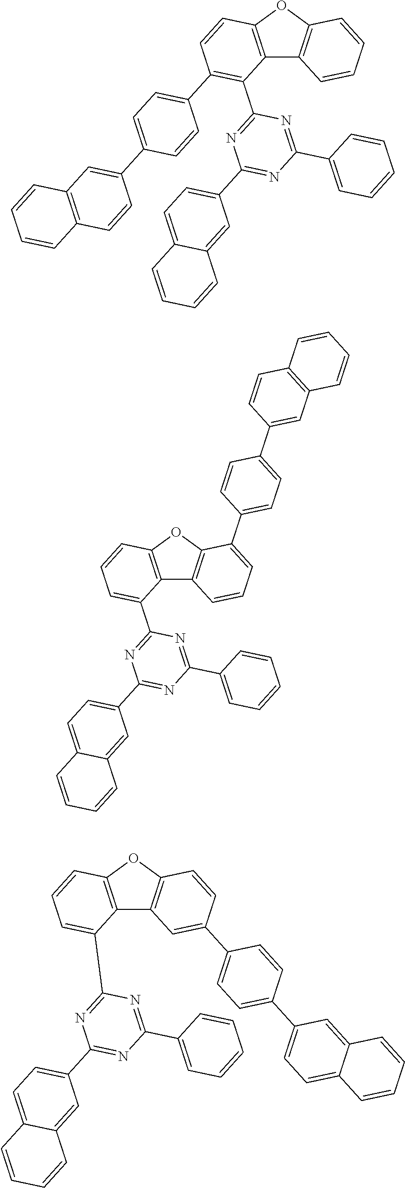 [SMILES: c1ccc(-c2nc(-c3ccc4ccccc4c3)nc(-c3c(-c4ccc(-c5ccc6ccccc6c5)cc4)ccc4oc5ccccc5c34)n2)cc1 ; c1ccc(-c2nc(-c3ccc4ccccc4c3)nc(-c3cccc4oc5c(-c6ccc(-c7ccc8ccccc8c7)cc6)cccc5c34)n2)cc1 ; c1ccc(-c2nc(-c3ccc4ccccc4c3)nc(-c3cccc4oc5ccc(-c6ccc(-c7ccc8ccccc8c7)cc6)cc5c34)n2)cc1]

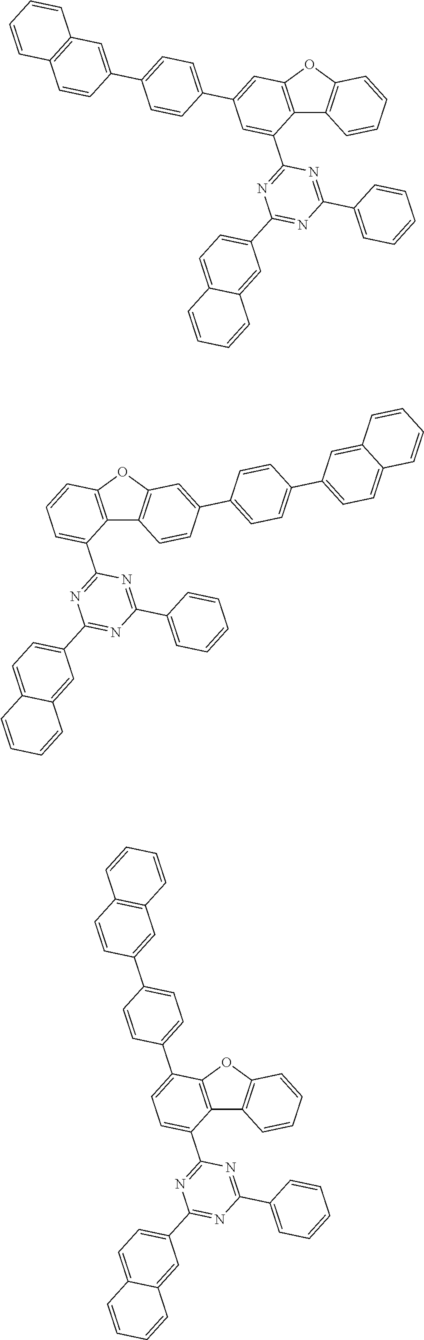 [SMILES: c1ccc(-c2nc(-c3ccc4ccccc4c3)nc(-c3cc(-c4ccc(-c5ccc6ccccc6c5)cc4)cc4oc5ccccc5c34)n2)cc1 ; c1ccc(-c2nc(-c3ccc4ccccc4c3)nc(-c3cccc4oc5cc(-c6ccc(-c7ccc8ccccc8c7)cc6)ccc5c34)n2)cc1 ; c1ccc(-c2nc(-c3ccc4ccccc4c3)nc(-c3ccc(-c4ccc(-c5ccc6ccccc6c5)cc4)c4oc5ccccc5c34)n2)cc1]

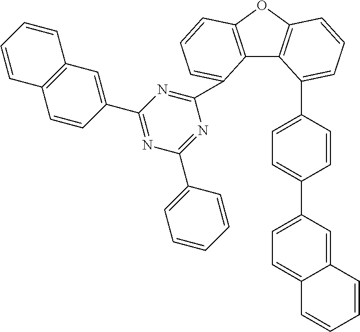 [SMILES: c1ccc(-c2nc(-c3ccc4ccccc4c3)nc(-c3cccc4oc5cccc(-c6ccc(-c7ccc8ccccc8c7)cc6)c5c34)n2)cc1]

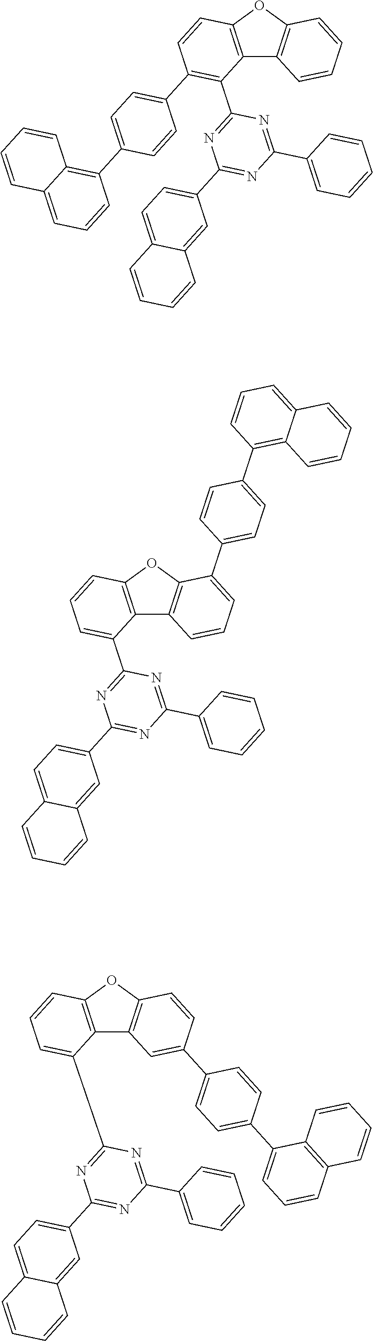 [SMILES: c1ccc(-c2nc(-c3ccc4ccccc4c3)nc(-c3c(-c4ccc(-c5cccc6ccccc56)cc4)ccc4oc5ccccc5c34)n2)cc1 ; c1ccc(-c2nc(-c3ccc4ccccc4c3)nc(-c3cccc4oc5c(-c6ccc(-c7cccc8ccccc78)cc6)cccc5c34)n2)cc1 ; c1ccc(-c2nc(-c3ccc4ccccc4c3)nc(-c3cccc4oc5ccc(-c6ccc(-c7cccc8ccccc78)cc6)cc5c34)n2)cc1]

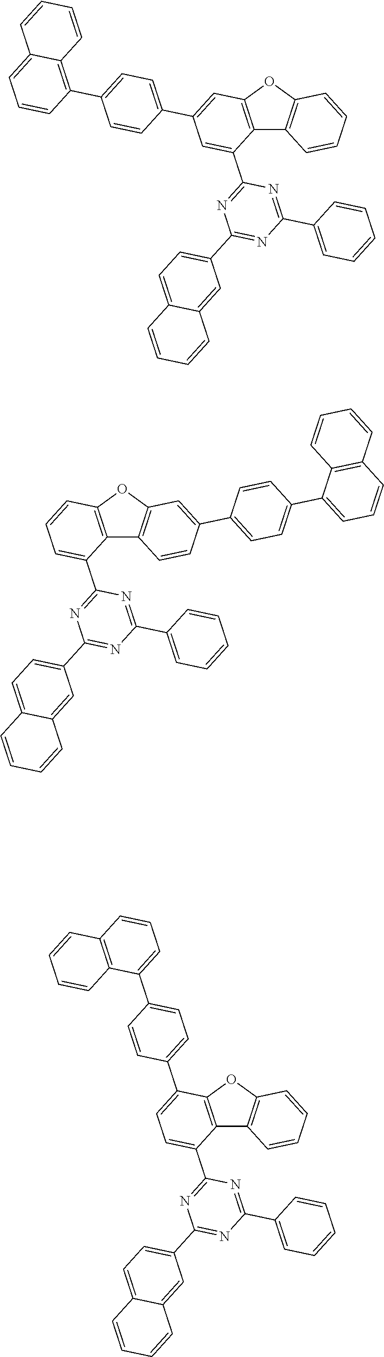 [SMILES: c1ccc(-c2nc(-c3ccc4ccccc4c3)nc(-c3cc(-c4ccc(-c5cccc6ccccc56)cc4)cc4oc5ccccc5c34)n2)cc1 ; c1ccc(-c2nc(-c3ccc4ccccc4c3)nc(-c3cccc4oc5cc(-c6ccc(-c7cccc8ccccc78)cc6)ccc5c34)n2)cc1 ; c1ccc(-c2nc(-c3ccc4ccccc4c3)nc(-c3ccc(-c4ccc(-c5cccc6ccccc56)cc4)c4oc5ccccc5c34)n2)cc1]

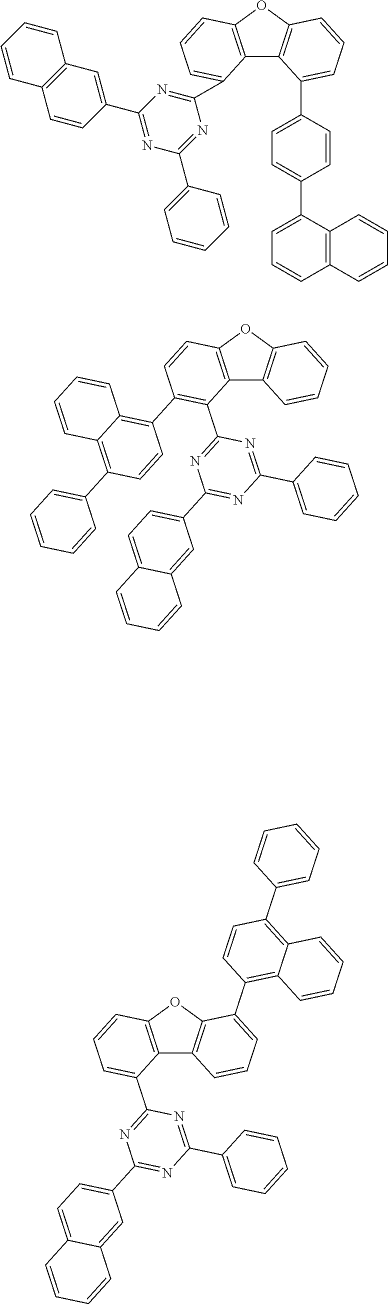 [SMILES: c1ccc(-c2nc(-c3ccc4ccccc4c3)nc(-c3cccc4oc5cccc(-c6ccc(-c7cccc8ccccc78)cc6)c5c34)n2)cc1 ; c1ccc(-c2nc(-c3ccc4ccccc4c3)nc(-c3c(-c4ccc(-c5ccccc5)c5ccccc45)ccc4oc5ccccc5c34)n2)cc1 ; c1ccc(-c2nc(-c3ccc4ccccc4c3)nc(-c3cccc4oc5c(-c6ccc(-c7ccccc7)c7ccccc67)cccc5c34)n2)cc1]

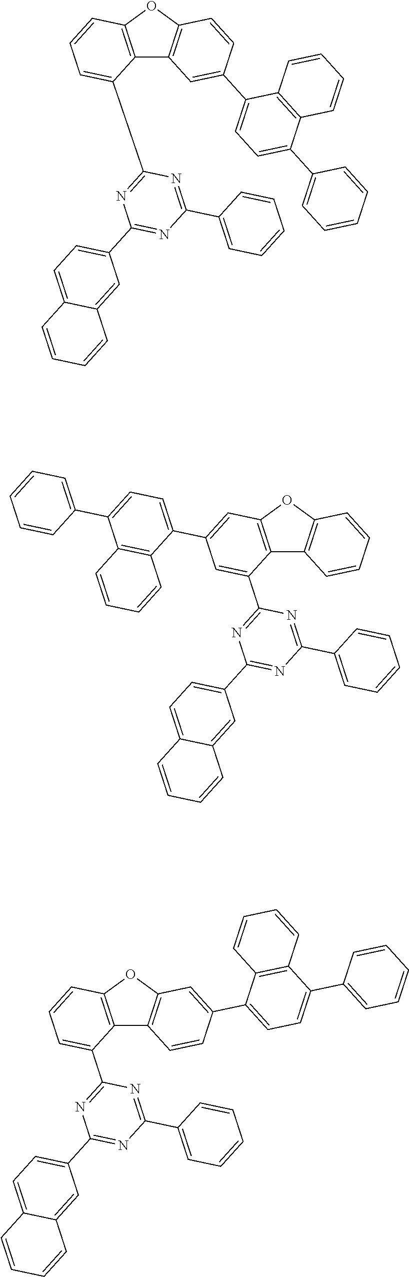 [SMILES: c1ccc(-c2nc(-c3ccc4ccccc4c3)nc(-c3cccc4oc5ccc(-c6ccc(-c7ccccc7)c7ccccc67)cc5c34)n2)cc1 ; c1ccc(-c2nc(-c3ccc4ccccc4c3)nc(-c3cc(-c4ccc(-c5ccccc5)c5ccccc45)cc4oc5ccccc5c34)n2)cc1 ; c1ccc(-c2nc(-c3ccc4ccccc4c3)nc(-c3cccc4oc5cc(-c6ccc(-c7ccccc7)c7ccccc67)ccc5c34)n2)cc1]

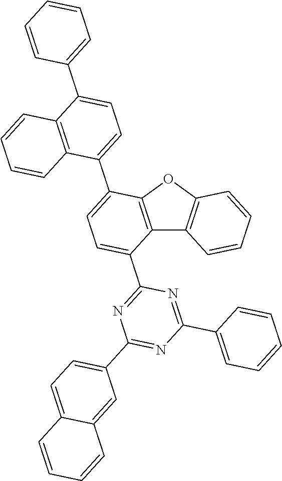 [SMILES: c1ccc(-c2nc(-c3ccc4ccccc4c3)nc(-c3ccc(-c4ccc(-c5ccccc5)c5ccccc45)c4oc5ccccc5c34)n2)cc1]

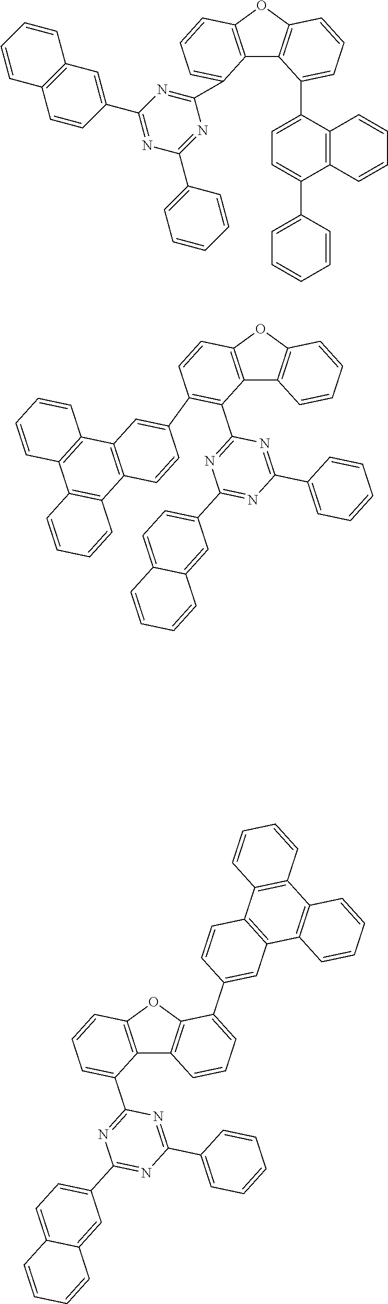 [SMILES: c1ccc(-c2nc(-c3ccc4ccccc4c3)nc(-c3cccc4oc5cccc(-c6ccc(-c7ccccc7)c7ccccc67)c5c34)n2)cc1 ; c1ccc(-c2nc(-c3ccc4ccccc4c3)nc(-c3c(-c4ccc5c6ccccc6c6ccccc6c5c4)ccc4oc5ccccc5c34)n2)cc1 ; c1ccc(-c2nc(-c3ccc4ccccc4c3)nc(-c3cccc4oc5c(-c6ccc7c8ccccc8c8ccccc8c7c6)cccc5c34)n2)cc1]

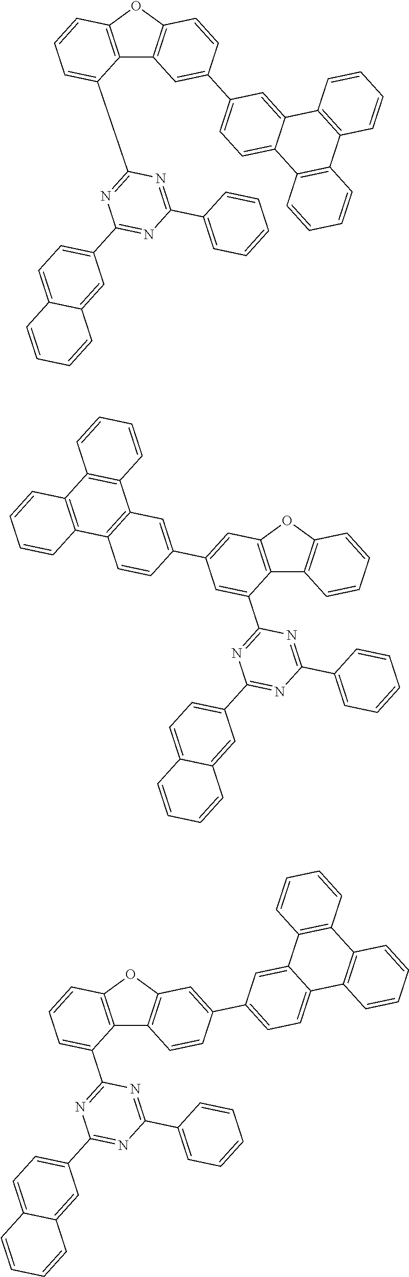 [SMILES: c1ccc(-c2nc(-c3ccc4ccccc4c3)nc(-c3cccc4oc5ccc(-c6ccc7c8ccccc8c8ccccc8c7c6)cc5c34)n2)cc1 ; c1ccc(-c2nc(-c3ccc4ccccc4c3)nc(-c3cc(-c4ccc5c6ccccc6c6ccccc6c5c4)cc4oc5ccccc5c34)n2)cc1 ; c1ccc(-c2nc(-c3ccc4ccccc4c3)nc(-c3cccc4oc5cc(-c6ccc7c8ccccc8c8ccccc8c7c6)ccc5c34)n2)cc1]

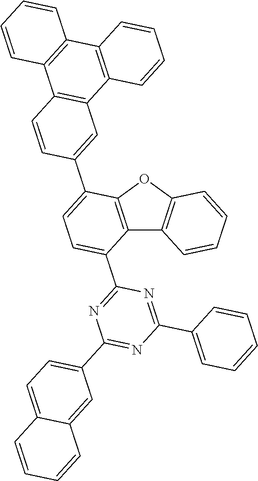 [SMILES: c1ccc(-c2nc(-c3ccc4ccccc4c3)nc(-c3ccc(-c4ccc5c6ccccc6c6ccccc6c5c4)c4oc5ccccc5c34)n2)cc1]

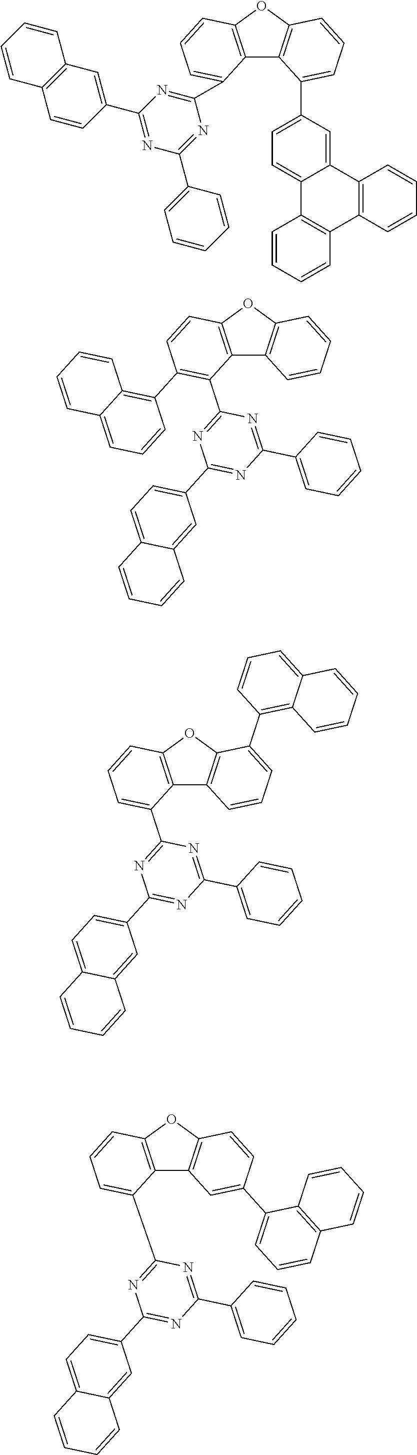

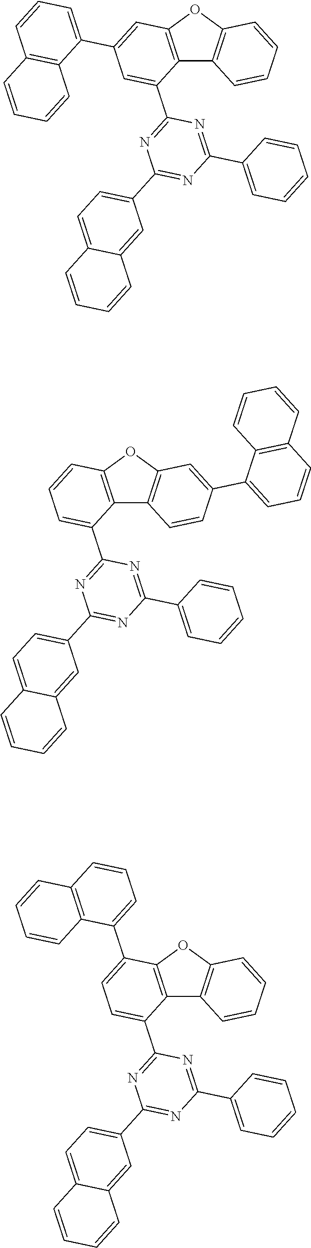 [SMILES: c1ccc(-c2nc(-c3ccc4ccccc4c3)nc(-c3cc(-c4cccc5ccccc45)cc4oc5ccccc5c34)n2)cc1 ; c1ccc(-c2nc(-c3ccc4ccccc4c3)nc(-c3cccc4oc5cc(-c6cccc7ccccc67)ccc5c34)n2)cc1 ; c1ccc(-c2nc(-c3ccc4ccccc4c3)nc(-c3ccc(-c4cccc5ccccc45)c4oc5ccccc5c34)n2)cc1]

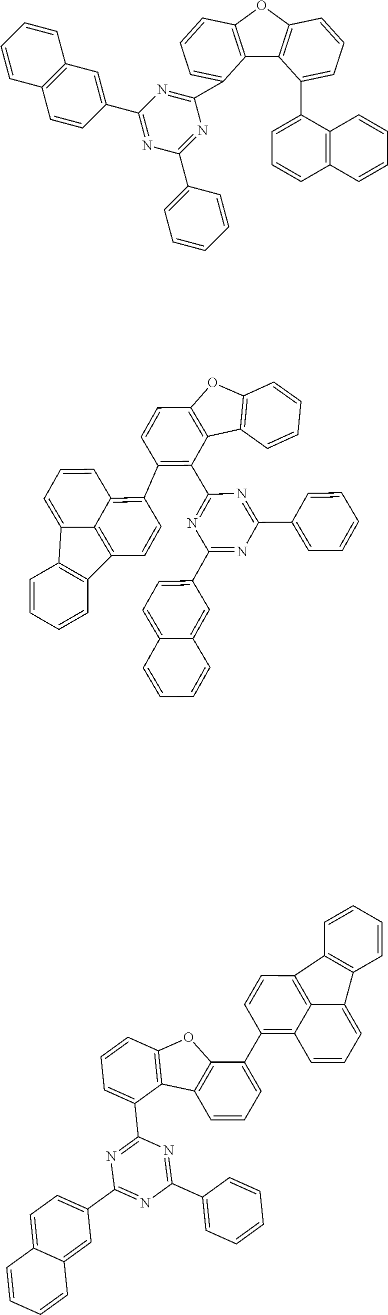 [SMILES: c1ccc(-c2nc(-c3ccc4ccccc4c3)nc(-c3cccc4oc5cccc(-c6cccc7ccccc67)c5c34)n2)cc1 ; c1ccc(-c2nc(-c3ccc4ccccc4c3)nc(-c3c(-c4ccc5c6c(cccc46)-c4ccccc4-5)ccc4oc5ccccc5c34)n2)cc1 ; c1ccc(-c2nc(-c3ccc4ccccc4c3)nc(-c3cccc4oc5c(-c6ccc7c8c(cccc68)-c6ccccc6-7)cccc5c34)n2)cc1]

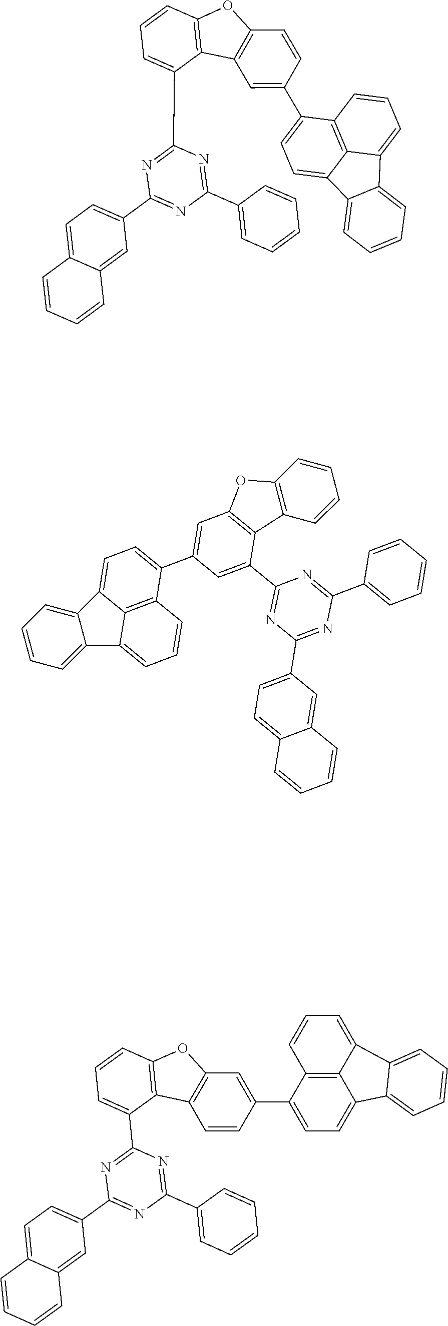 [SMILES: c1ccc(-c2nc(-c3ccc4ccccc4c3)nc(-c3cccc4oc5ccc(-c6ccc7c8c(cccc68)-c6ccccc6-7)cc5c34)n2)cc1 ; c1ccc(-c2nc(-c3ccc4ccccc4c3)nc(-c3cc(-c4ccc5c6c(cccc46)-c4ccccc4-5)cc4oc5ccccc5c34)n2)cc1 ; c1ccc(-c2nc(-c3ccc4ccccc4c3)nc(-c3cccc4oc5cc(-c6ccc7c8c(cccc68)-c6ccccc6-7)ccc5c34)n2)cc1]

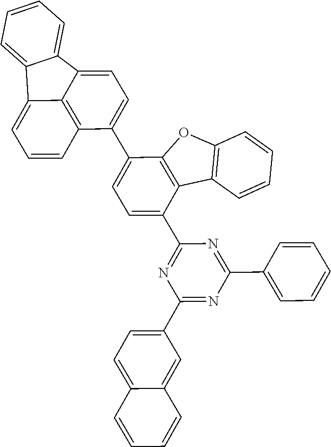 [SMILES: c1ccc(-c2nc(-c3ccc4ccccc4c3)nc(-c3ccc(-c4ccc5c6c(cccc46)-c4ccccc4-5)c4oc5ccccc5c34)n2)cc1]

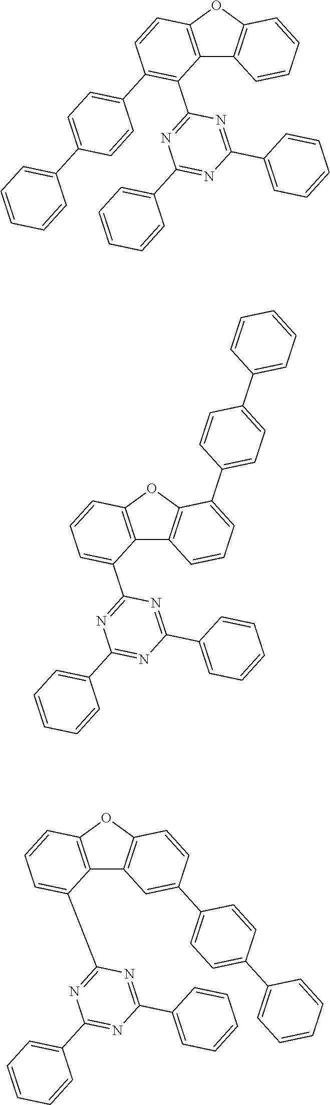 [SMILES: c1ccc(-c2ccc(-c3ccc4oc5ccccc5c4c3-c3nc(-c4ccccc4)nc(-c4ccccc4)n3)cc2)cc1 ; c1ccc(-c2ccc(-c3cccc4c3oc3cccc(-c5nc(-c6ccccc6)nc(-c6ccccc6)n5)c34)cc2)cc1 ; c1ccc(-c2ccc(-c3ccc4oc5cccc(-c6nc(-c7ccccc7)nc(-c7ccccc7)n6)c5c4c3)cc2)cc1]

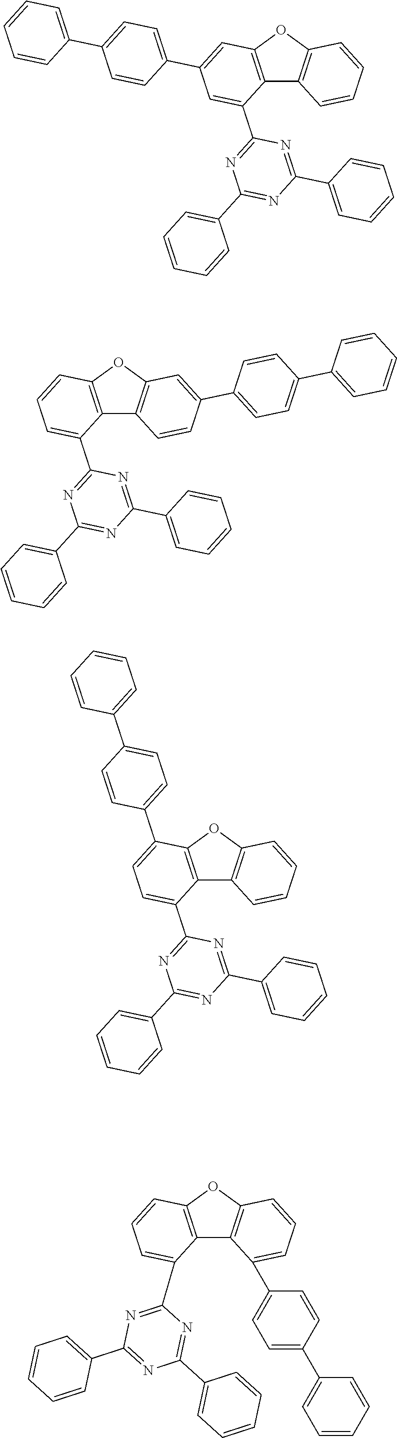 [SMILES: c1ccc(-c2ccc(-c3cc(-c4nc(-c5ccccc5)nc(-c5ccccc5)n4)c4c(c3)oc3ccccc34)cc2)cc1 ; c1ccc(-c2ccc(-c3ccc4c(c3)oc3cccc(-c5nc(-c6ccccc6)nc(-c6ccccc6)n5)c34)cc2)cc1 ; c1ccc(-c2ccc(-c3ccc(-c4nc(-c5ccccc5)nc(-c5ccccc5)n4)c4c3oc3ccccc34)cc2)cc1 ; c1ccc(-c2ccc(-c3cccc4oc5cccc(-c6nc(-c7ccccc7)nc(-c7ccccc7)n6)c5c34)cc2)cc1]

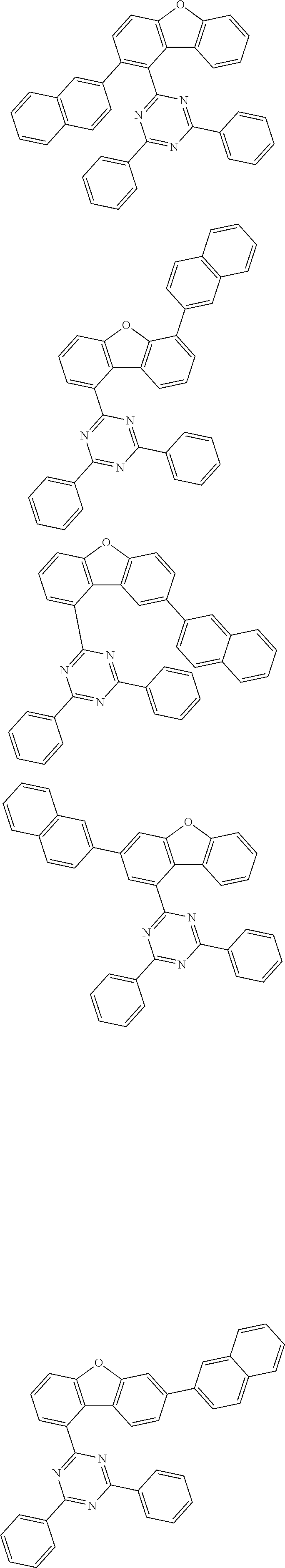

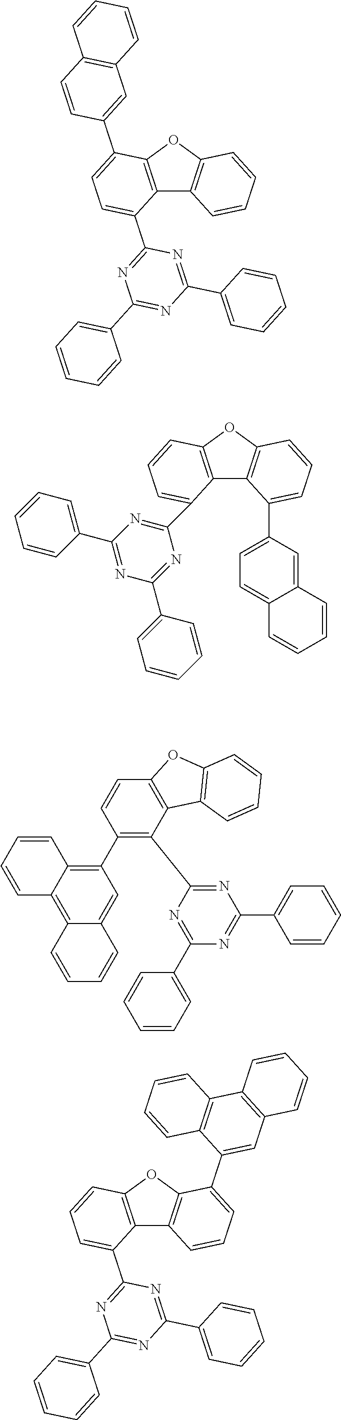

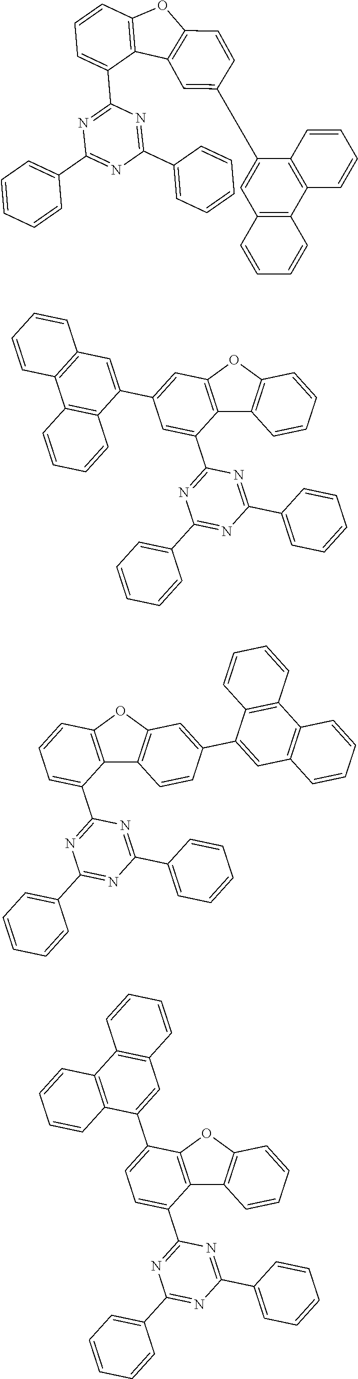 [SMILES: c1ccc(-c2nc(-c3ccccc3)nc(-c3cccc4oc5ccc(-c6cc7ccccc7c7ccccc67)cc5c34)n2)cc1 ; c1ccc(-c2nc(-c3ccccc3)nc(-c3cc(-c4cc5ccccc5c5ccccc45)cc4oc5ccccc5c34)n2)cc1 ; c1ccc(-c2nc(-c3ccccc3)nc(-c3cccc4oc5cc(-c6cc7ccccc7c7ccccc67)ccc5c34)n2)cc1 ; c1ccc(-c2nc(-c3ccccc3)nc(-c3ccc(-c4cc5ccccc5c5ccccc45)c4oc5ccccc5c34)n2)cc1]

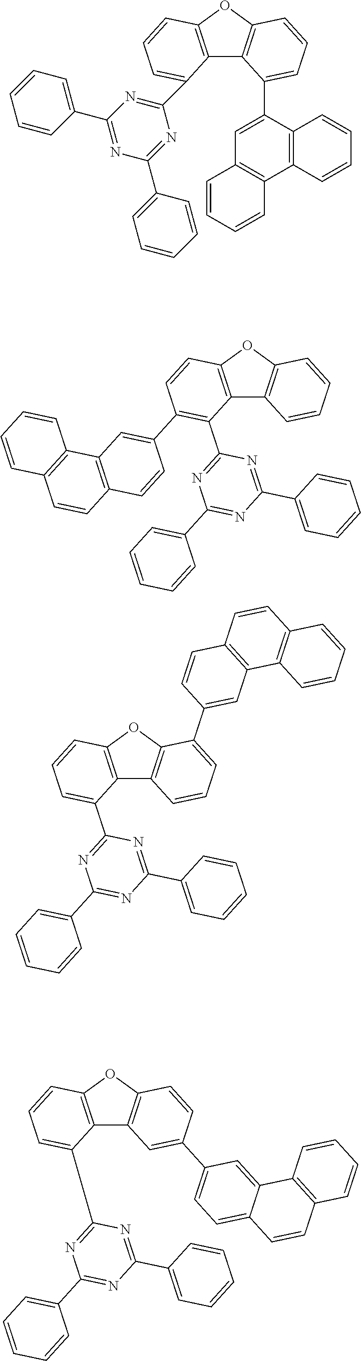 [SMILES: c1ccc(-c2nc(-c3ccccc3)nc(-c3cccc4oc5cccc(-c6cc7ccccc7c7ccccc67)c5c34)n2)cc1 ; c1ccc(-c2nc(-c3ccccc3)nc(-c3c(-c4ccc5ccc6ccccc6c5c4)ccc4oc5ccccc5c34)n2)cc1 ; c1ccc(-c2nc(-c3ccccc3)nc(-c3cccc4oc5c(-c6ccc7ccc8ccccc8c7c6)cccc5c34)n2)cc1 ; c1ccc(-c2nc(-c3ccccc3)nc(-c3cccc4oc5ccc(-c6ccc7ccc8ccccc8c7c6)cc5c34)n2)cc1]

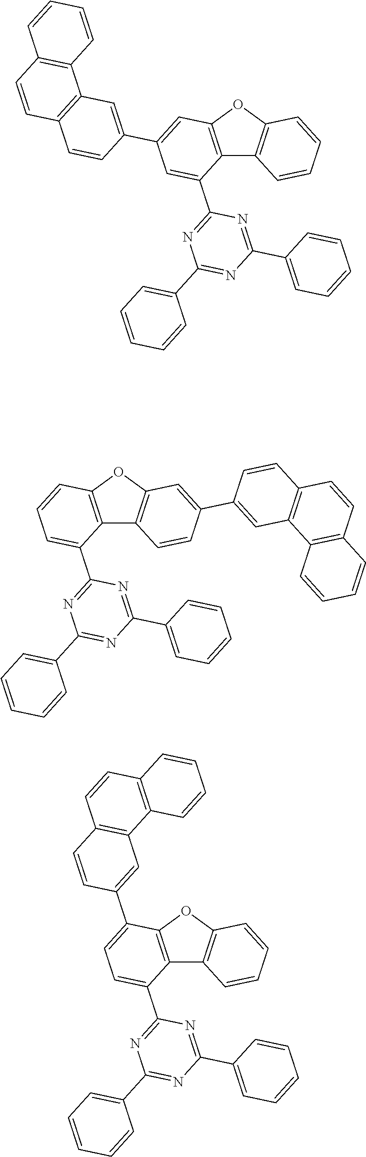 [SMILES: c1ccc(-c2nc(-c3ccccc3)nc(-c3cc(-c4ccc5ccc6ccccc6c5c4)cc4oc5ccccc5c34)n2)cc1 ; c1ccc(-c2nc(-c3ccccc3)nc(-c3cccc4oc5cc(-c6ccc7ccc8ccccc8c7c6)ccc5c34)n2)cc1 ; c1ccc(-c2nc(-c3ccccc3)nc(-c3ccc(-c4ccc5ccc6ccccc6c5c4)c4oc5ccccc5c34)n2)cc1]

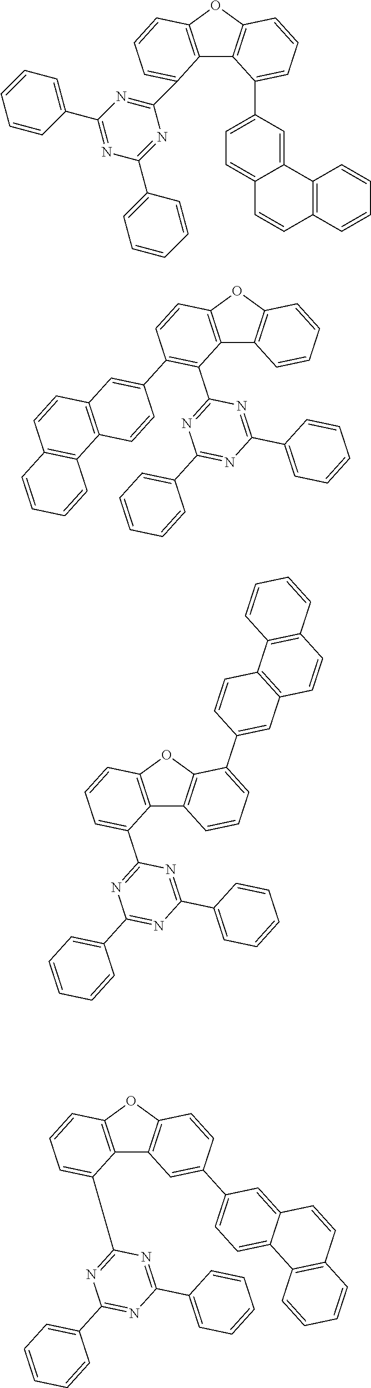 [SMILES: c1ccc(-c2nc(-c3ccccc3)nc(-c3cccc4oc5cccc(-c6ccc7ccc8ccccc8c7c6)c5c34)n2)cc1 ; c1ccc(-c2nc(-c3ccccc3)nc(-c3c(-c4ccc5c(ccc6ccccc65)c4)ccc4oc5ccccc5c34)n2)cc1 ; c1ccc(-c2nc(-c3ccccc3)nc(-c3cccc4oc5c(-c6ccc7c(ccc8ccccc87)c6)cccc5c34)n2)cc1 ; c1ccc(-c2nc(-c3ccccc3)nc(-c3cccc4oc5ccc(-c6ccc7c(ccc8ccccc87)c6)cc5c34)n2)cc1]

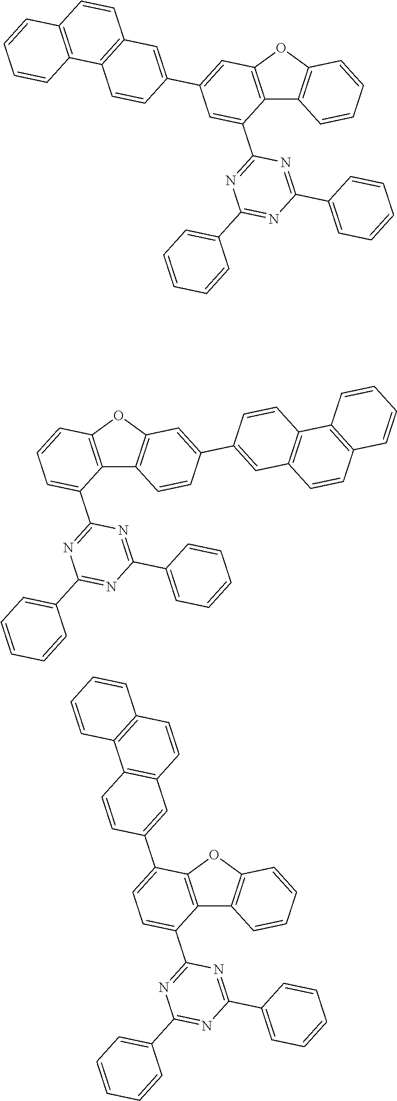 [SMILES: c1ccc(-c2nc(-c3ccccc3)nc(-c3cc(-c4ccc5c(ccc6ccccc65)c4)cc4oc5ccccc5c34)n2)cc1 ; c1ccc(-c2nc(-c3ccccc3)nc(-c3cccc4oc5cc(-c6ccc7c(ccc8ccccc87)c6)ccc5c34)n2)cc1 ; c1ccc(-c2nc(-c3ccccc3)nc(-c3ccc(-c4ccc5c(ccc6ccccc65)c4)c4oc5ccccc5c34)n2)cc1]

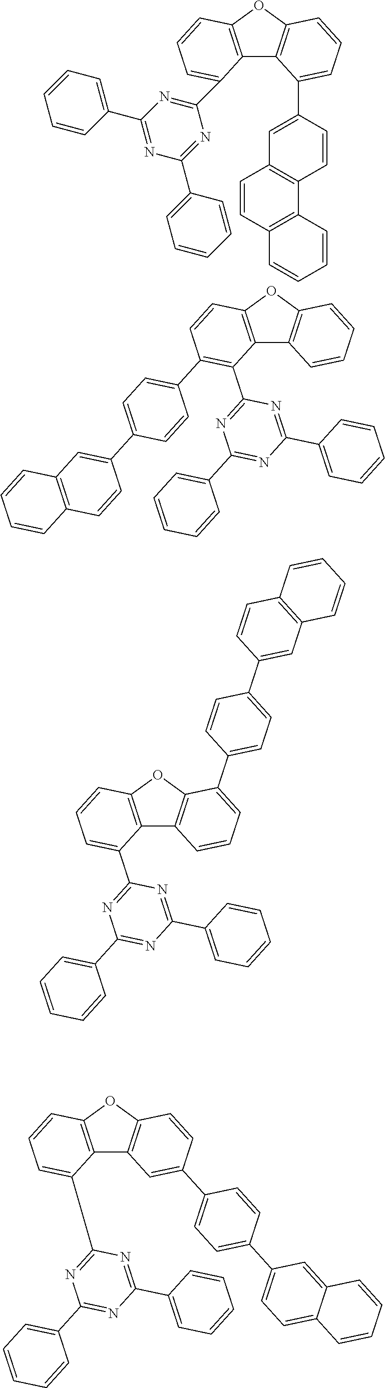

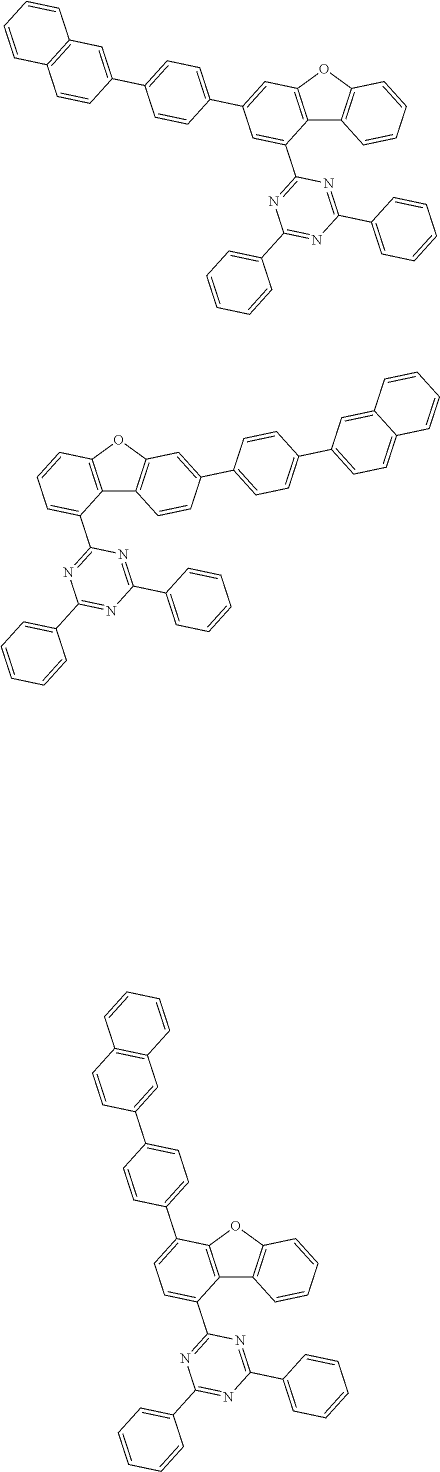 [SMILES: c1ccc(-c2nc(-c3ccccc3)nc(-c3cc(-c4ccc(-c5ccc6ccccc6c5)cc4)cc4oc5ccccc5c34)n2)cc1 ; c1ccc(-c2nc(-c3ccccc3)nc(-c3cccc4oc5cc(-c6ccc(-c7ccc8ccccc8c7)cc6)ccc5c34)n2)cc1 ; c1ccc(-c2nc(-c3ccccc3)nc(-c3ccc(-c4ccc(-c5ccc6ccccc6c5)cc4)c4oc5ccccc5c34)n2)cc1]

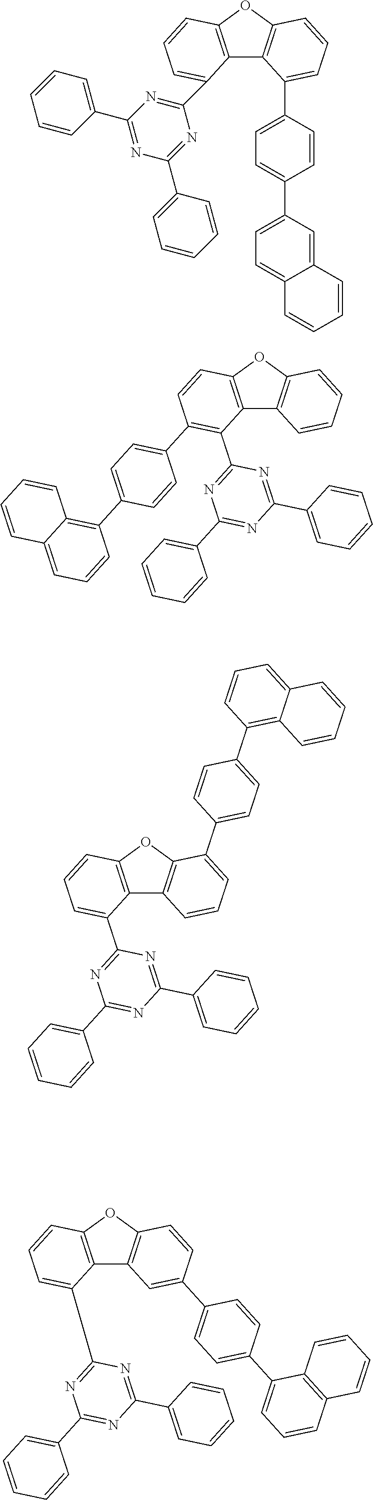

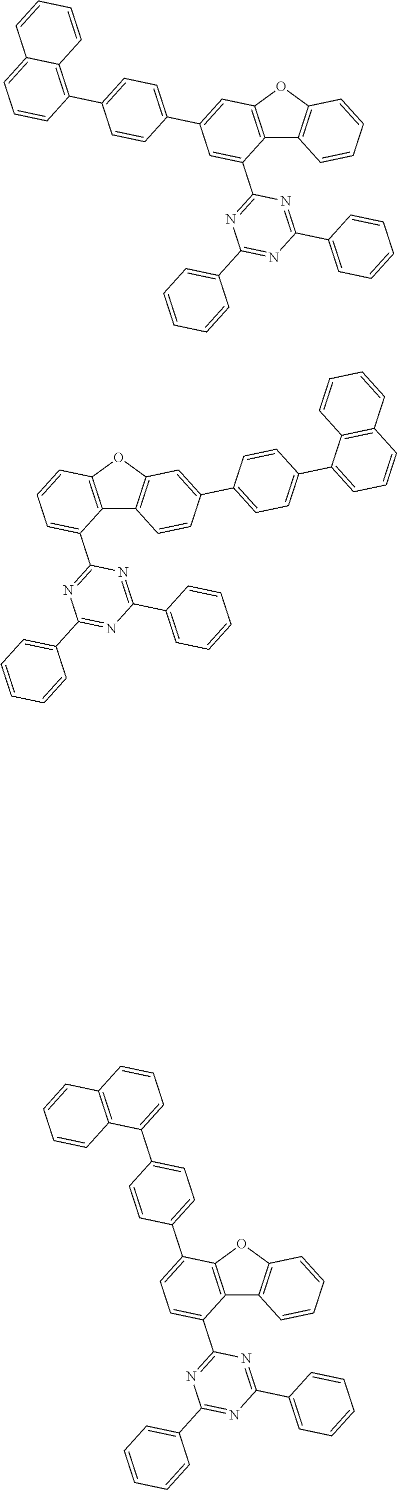 [SMILES: c1ccc(-c2nc(-c3ccccc3)nc(-c3cc(-c4ccc(-c5cccc6ccccc56)cc4)cc4oc5ccccc5c34)n2)cc1 ; c1ccc(-c2nc(-c3ccccc3)nc(-c3cccc4oc5cc(-c6ccc(-c7cccc8ccccc78)cc6)ccc5c34)n2)cc1 ; c1ccc(-c2nc(-c3ccccc3)nc(-c3ccc(-c4ccc(-c5cccc6ccccc56)cc4)c4oc5ccccc5c34)n2)cc1]

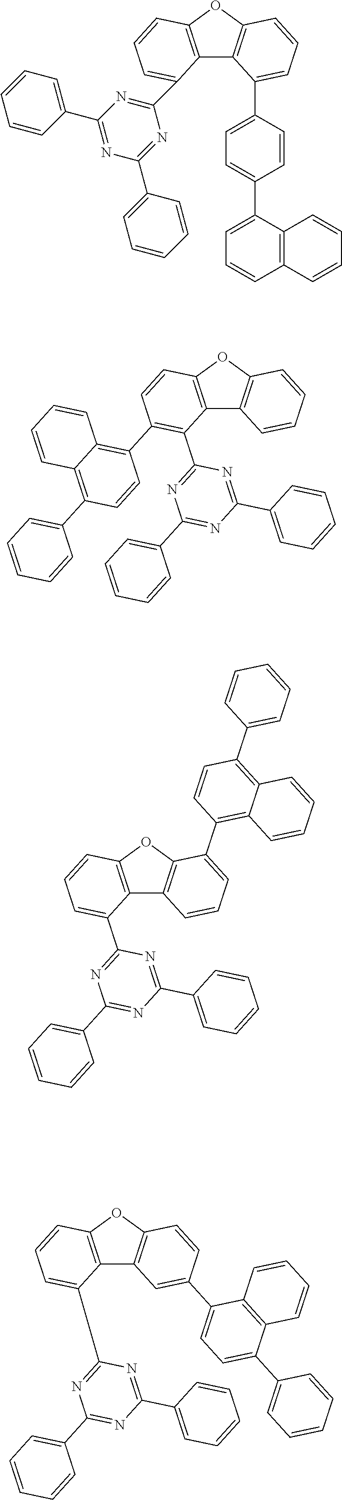 [SMILES: c1ccc(-c2nc(-c3ccccc3)nc(-c3cccc4oc5cccc(-c6ccc(-c7cccc8ccccc78)cc6)c5c34)n2)cc1 ; c1ccc(-c2nc(-c3ccccc3)nc(-c3c(-c4ccc(-c5ccccc5)c5ccccc45)ccc4oc5ccccc5c34)n2)cc1 ; c1ccc(-c2nc(-c3ccccc3)nc(-c3cccc4oc5c(-c6ccc(-c7ccccc7)c7ccccc67)cccc5c34)n2)cc1 ; c1ccc(-c2nc(-c3ccccc3)nc(-c3cccc4oc5ccc(-c6ccc(-c7ccccc7)c7ccccc67)cc5c34)n2)cc1]

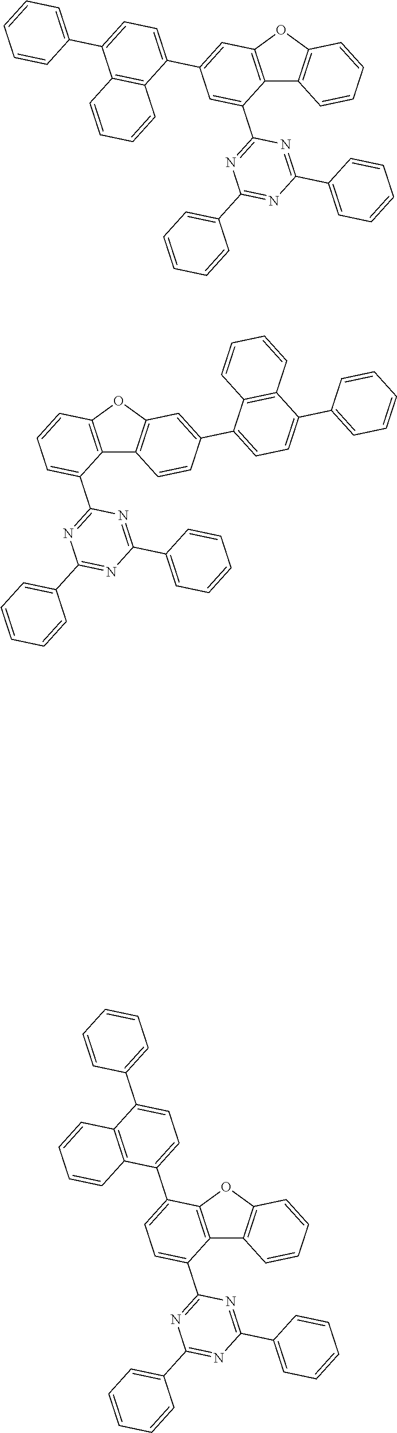 [SMILES: c1ccc(-c2nc(-c3ccccc3)nc(-c3cc(-c4ccc(-c5ccccc5)c5ccccc45)cc4oc5ccccc5c34)n2)cc1 ; c1ccc(-c2nc(-c3ccccc3)nc(-c3cccc4oc5cc(-c6ccc(-c7ccccc7)c7ccccc67)ccc5c34)n2)cc1 ; c1ccc(-c2nc(-c3ccccc3)nc(-c3ccc(-c4ccc(-c5ccccc5)c5ccccc45)c4oc5ccccc5c34)n2)cc1]

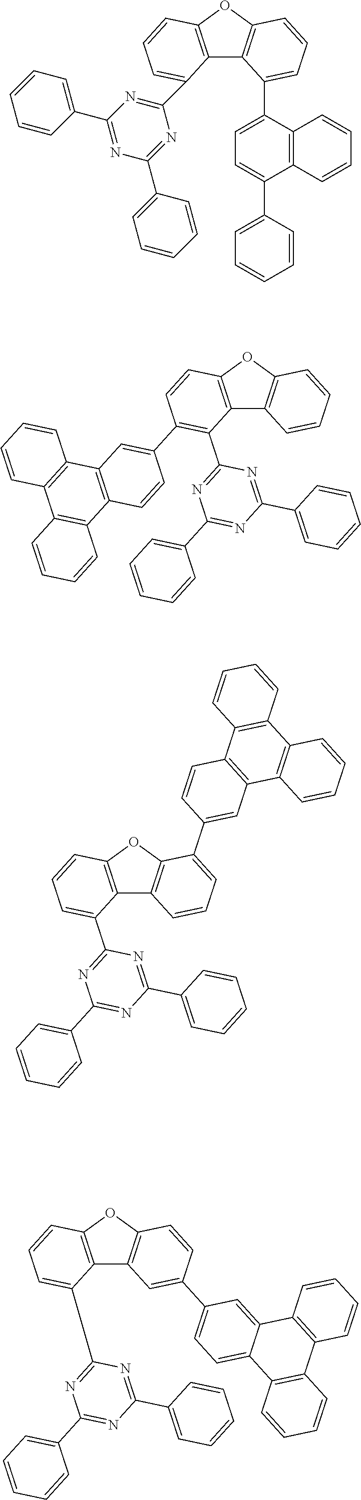 [SMILES: c1ccc(-c2nc(-c3ccccc3)nc(-c3cccc4oc5cccc(-c6ccc(-c7ccccc7)c7ccccc67)c5c34)n2)cc1 ; c1ccc(-c2nc(-c3ccccc3)nc(-c3c(-c4ccc5c6ccccc6c6ccccc6c5c4)ccc4oc5ccccc5c34)n2)cc1 ; c1ccc(-c2nc(-c3ccccc3)nc(-c3cccc4oc5c(-c6ccc7c8ccccc8c8ccccc8c7c6)cccc5c34)n2)cc1 ; c1ccc(-c2nc(-c3ccccc3)nc(-c3cccc4oc5ccc(-c6ccc7c8ccccc8c8ccccc8c7c6)cc5c34)n2)cc1]

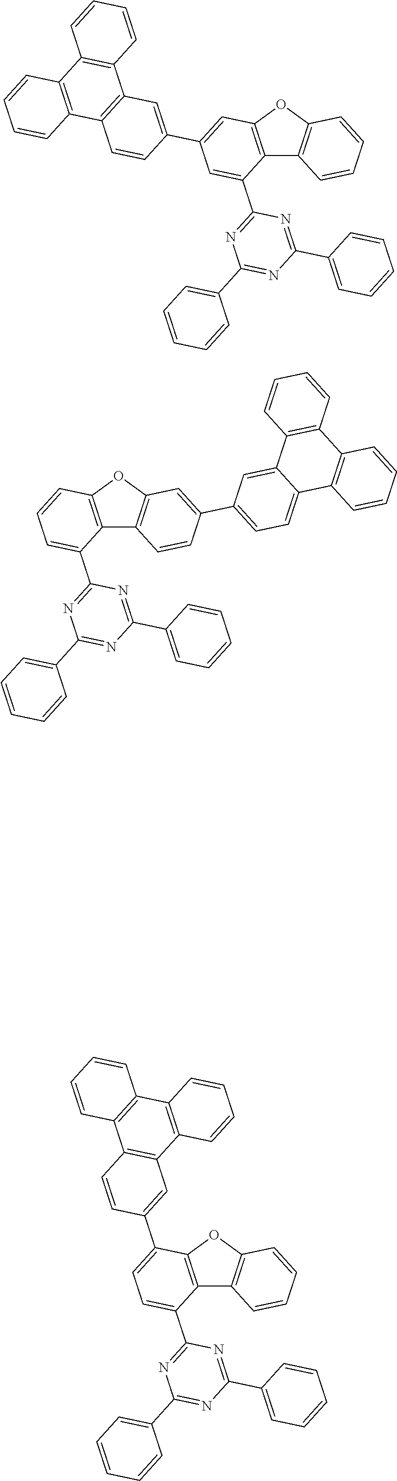 [SMILES: c1ccc(-c2nc(-c3ccccc3)nc(-c3cc(-c4ccc5c6ccccc6c6ccccc6c5c4)cc4oc5ccccc5c34)n2)cc1 ; c1ccc(-c2nc(-c3ccccc3)nc(-c3cccc4oc5cc(-c6ccc7c8ccccc8c8ccccc8c7c6)ccc5c34)n2)cc1 ; c1ccc(-c2nc(-c3ccccc3)nc(-c3ccc(-c4ccc5c6ccccc6c6ccccc6c5c4)c4oc5ccccc5c34)n2)cc1]

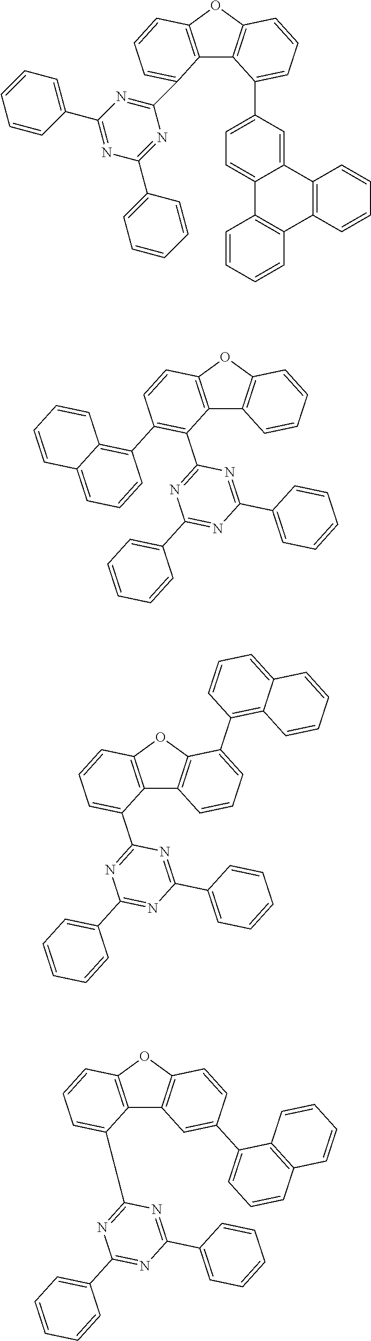 [SMILES: c1ccc(-c2nc(-c3ccccc3)nc(-c3cccc4oc5cccc(-c6ccc7c8ccccc8c8ccccc8c7c6)c5c34)n2)cc1 ; c1ccc(-c2nc(-c3ccccc3)nc(-c3c(-c4cccc5ccccc45)ccc4oc5ccccc5c34)n2)cc1 ; c1ccc(-c2nc(-c3ccccc3)nc(-c3cccc4oc5c(-c6cccc7ccccc67)cccc5c34)n2)cc1 ; c1ccc(-c2nc(-c3ccccc3)nc(-c3cccc4oc5ccc(-c6cccc7ccccc67)cc5c34)n2)cc1]

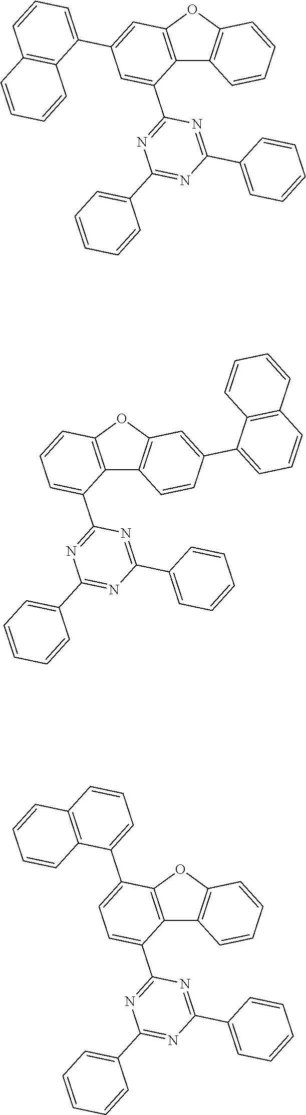 [SMILES: c1ccc(-c2nc(-c3ccccc3)nc(-c3cc(-c4cccc5ccccc45)cc4oc5ccccc5c34)n2)cc1 ; c1ccc(-c2nc(-c3ccccc3)nc(-c3cccc4oc5cc(-c6cccc7ccccc67)ccc5c34)n2)cc1 ; c1ccc(-c2nc(-c3ccccc3)nc(-c3ccc(-c4cccc5ccccc45)c4oc5ccccc5c34)n2)cc1]

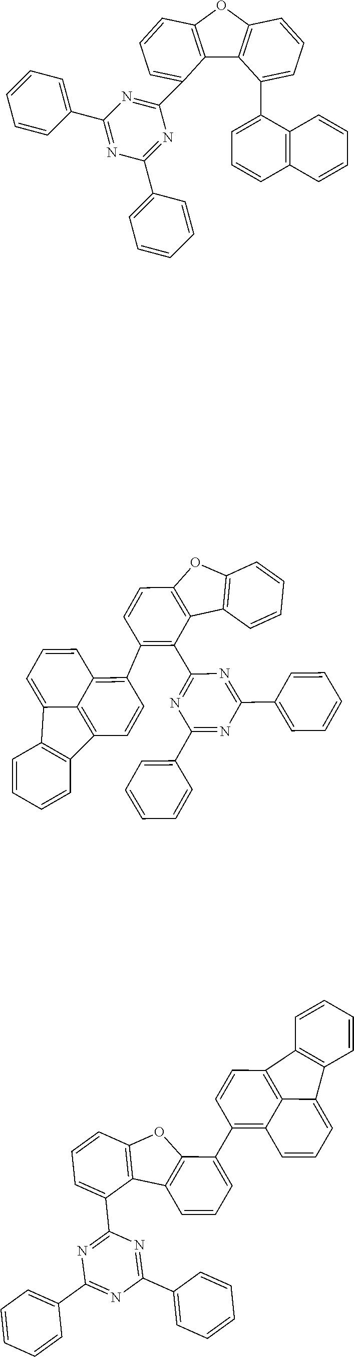 [SMILES: c1ccc(-c2nc(-c3ccccc3)nc(-c3cccc4oc5cccc(-c6cccc7ccccc67)c5c34)n2)cc1 ; c1ccc(-c2nc(-c3ccccc3)nc(-c3c(-c4ccc5c6c(cccc46)-c4ccccc4-5)ccc4oc5ccccc5c34)n2)cc1 ; c1ccc(-c2nc(-c3ccccc3)nc(-c3cccc4oc5c(-c6ccc7c8c(cccc68)-c6ccccc6-7)cccc5c34)n2)cc1]

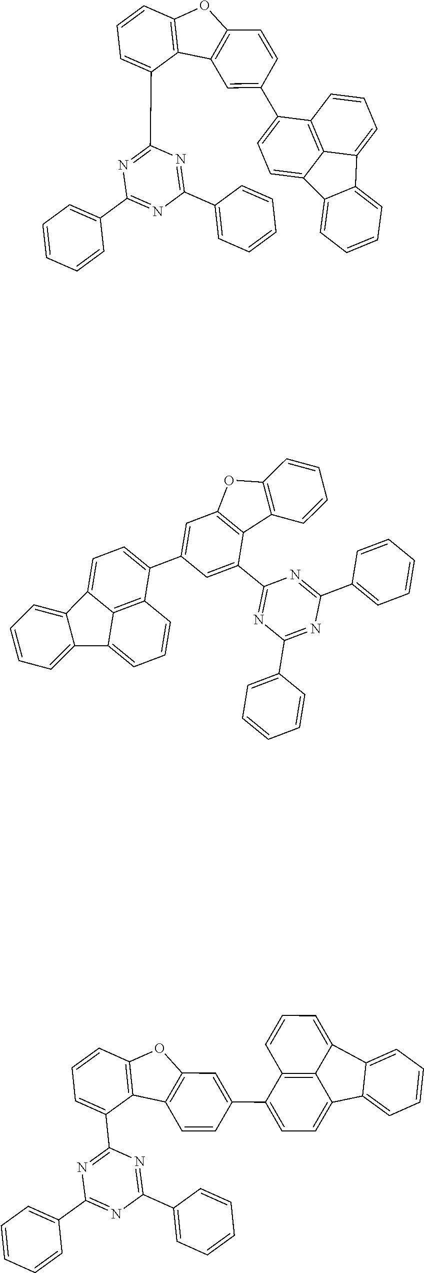 [SMILES: c1ccc(-c2nc(-c3ccccc3)nc(-c3cccc4oc5ccc(-c6ccc7c8c(cccc68)-c6ccccc6-7)cc5c34)n2)cc1 ; c1ccc(-c2nc(-c3ccccc3)nc(-c3cc(-c4ccc5c6c(cccc46)-c4ccccc4-5)cc4oc5ccccc5c34)n2)cc1 ; c1ccc(-c2nc(-c3ccccc3)nc(-c3cccc4oc5cc(-c6ccc7c8c(cccc68)-c6ccccc6-7)ccc5c34)n2)cc1]

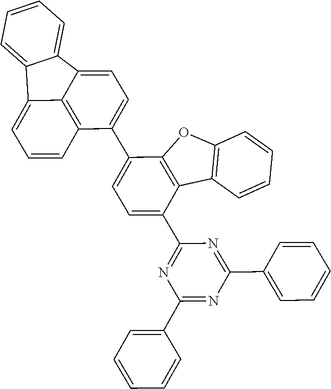 [SMILES: c1ccc(-c2nc(-c3ccccc3)nc(-c3ccc(-c4ccc5c6c(cccc46)-c4ccccc4-5)c4oc5ccccc5c34)n2)cc1]

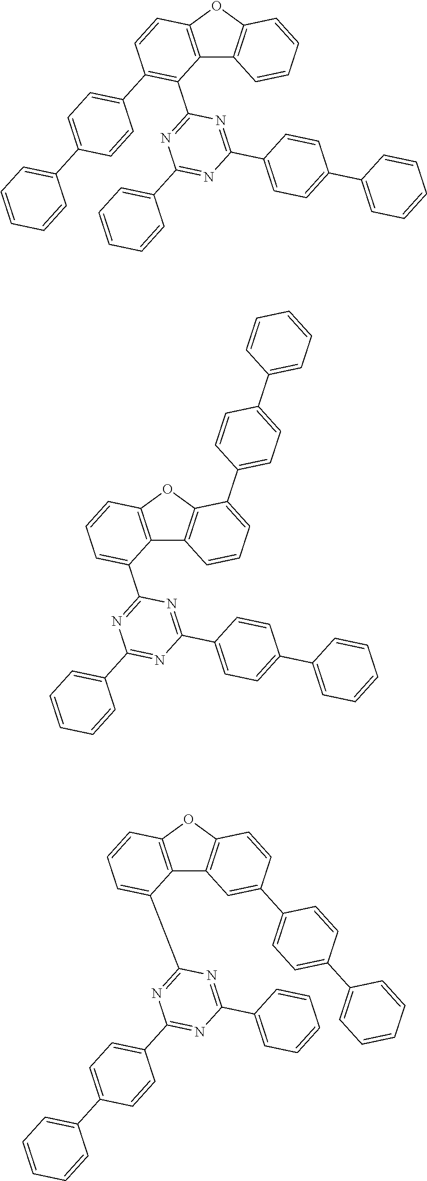 [SMILES: c1ccc(-c2ccc(-c3nc(-c4ccccc4)nc(-c4c(-c5ccc(-c6ccccc6)cc5)ccc5oc6ccccc6c45)n3)cc2)cc1 ; c1ccc(-c2ccc(-c3nc(-c4ccccc4)nc(-c4cccc5oc6c(-c7ccc(-c8ccccc8)cc7)cccc6c45)n3)cc2)cc1 ; c1ccc(-c2ccc(-c3ccc4oc5cccc(-c6nc(-c7ccccc7)nc(-c7ccc(-c8ccccc8)cc7)n6)c5c4c3)cc2)cc1]

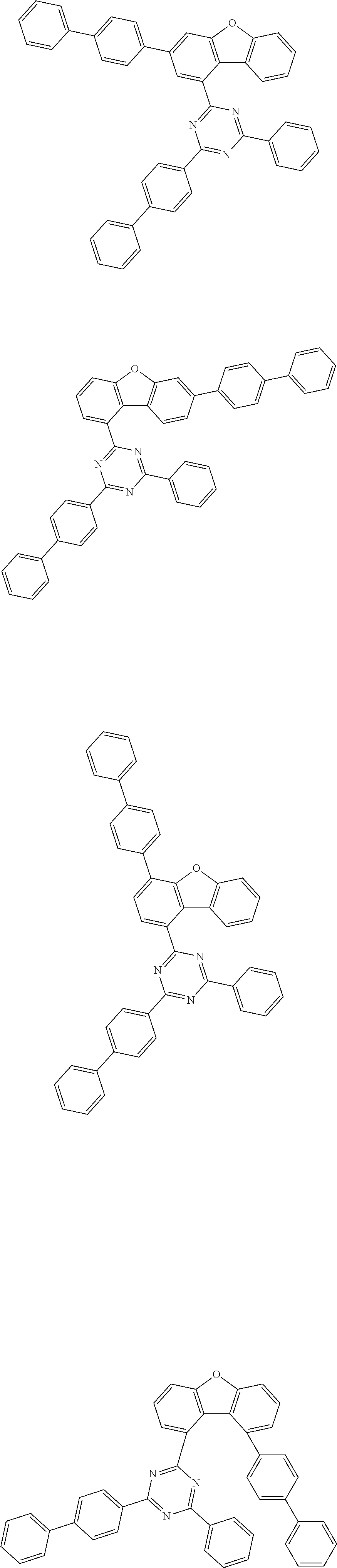 [SMILES: c1ccc(-c2ccc(-c3cc(-c4nc(-c5ccccc5)nc(-c5ccc(-c6ccccc6)cc5)n4)c4c(c3)oc3ccccc34)cc2)cc1 ; c1ccc(-c2ccc(-c3ccc4c(c3)oc3cccc(-c5nc(-c6ccccc6)nc(-c6ccc(-c7ccccc7)cc6)n5)c34)cc2)cc1 ; c1ccc(-c2ccc(-c3nc(-c4ccccc4)nc(-c4ccc(-c5ccc(-c6ccccc6)cc5)c5oc6ccccc6c45)n3)cc2)cc1 ; c1ccc(-c2ccc(-c3nc(-c4ccccc4)nc(-c4cccc5oc6cccc(-c7ccc(-c8ccccc8)cc7)c6c45)n3)cc2)cc1]

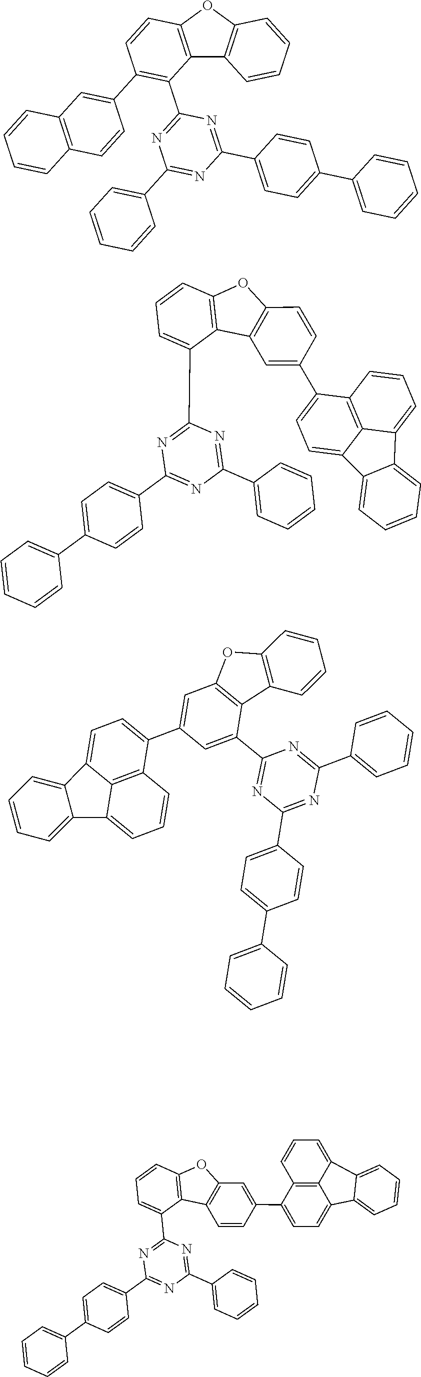 [SMILES: c1ccc(-c2ccc(-c3nc(-c4ccccc4)nc(-c4c(-c5ccc6ccccc6c5)ccc5oc6ccccc6c45)n3)cc2)cc1 ; c1ccc(-c2ccc(-c3nc(-c4ccccc4)nc(-c4cccc5oc6ccc(-c7ccc8c9c(cccc79)-c7ccccc7-8)cc6c45)n3)cc2)cc1 ; c1ccc(-c2ccc(-c3nc(-c4ccccc4)nc(-c4cc(-c5ccc6c7c(cccc57)-c5ccccc5-6)cc5oc6ccccc6c45)n3)cc2)cc1 ; c1ccc(-c2ccc(-c3nc(-c4ccccc4)nc(-c4cccc5oc6cc(-c7ccc8c9c(cccc79)-c7ccccc7-8)ccc6c45)n3)cc2)cc1]

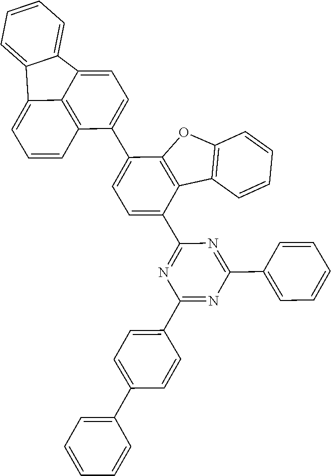 [SMILES: c1ccc(-c2ccc(-c3nc(-c4ccccc4)nc(-c4ccc(-c5ccc6c7c(cccc57)-c5ccccc5-6)c5oc6ccccc6c45)n3)cc2)cc1]

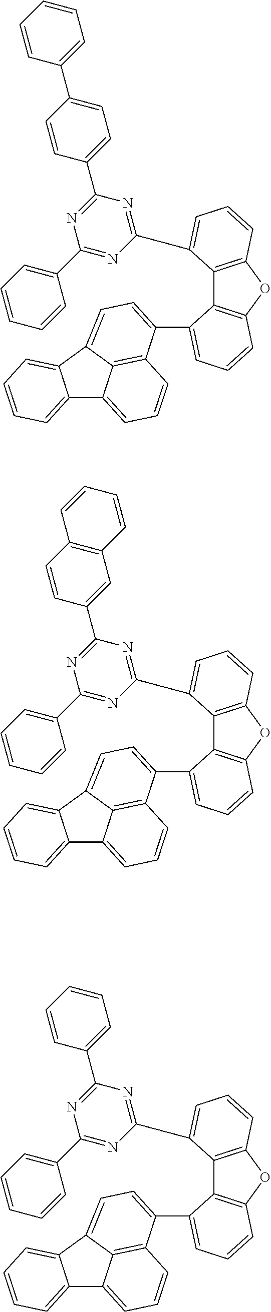 [SMILES: c1ccc(-c2ccc(-c3nc(-c4ccccc4)nc(-c4cccc5oc6cccc(-c7ccc8c9c(cccc79)-c7ccccc7-8)c6c45)n3)cc2)cc1 ; c1ccc(-c2nc(-c3ccc4ccccc4c3)nc(-c3cccc4oc5cccc(-c6ccc7c8c(cccc68)-c6ccccc6-7)c5c34)n2)cc1 ; c1ccc(-c2nc(-c3ccccc3)nc(-c3cccc4oc5cccc(-c6ccc7c8c(cccc68)-c6ccccc6-7)c5c34)n2)cc1]

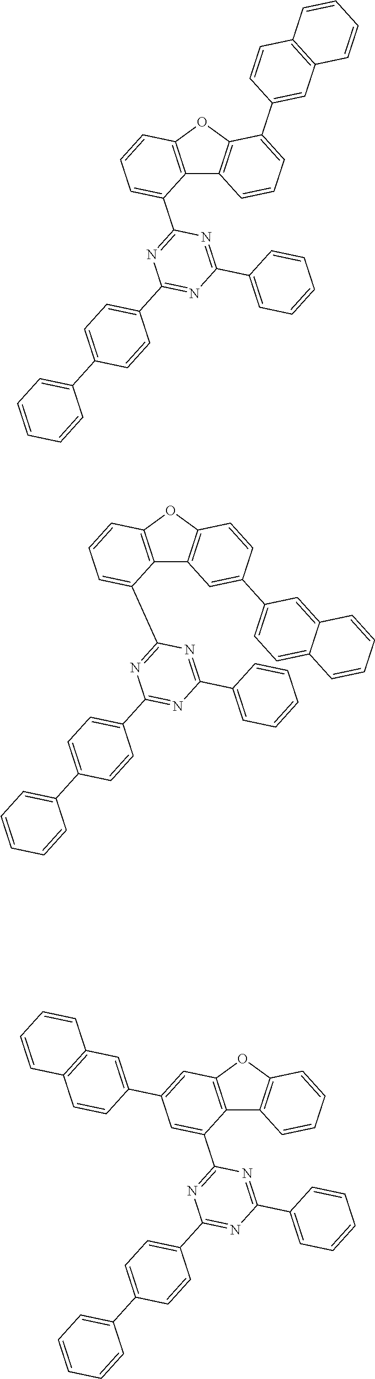 [SMILES: c1ccc(-c2ccc(-c3nc(-c4ccccc4)nc(-c4cccc5oc6c(-c7ccc8ccccc8c7)cccc6c45)n3)cc2)cc1 ; c1ccc(-c2ccc(-c3nc(-c4ccccc4)nc(-c4cccc5oc6ccc(-c7ccc8ccccc8c7)cc6c45)n3)cc2)cc1 ; c1ccc(-c2ccc(-c3nc(-c4ccccc4)nc(-c4cc(-c5ccc6ccccc6c5)cc5oc6ccccc6c45)n3)cc2)cc1]

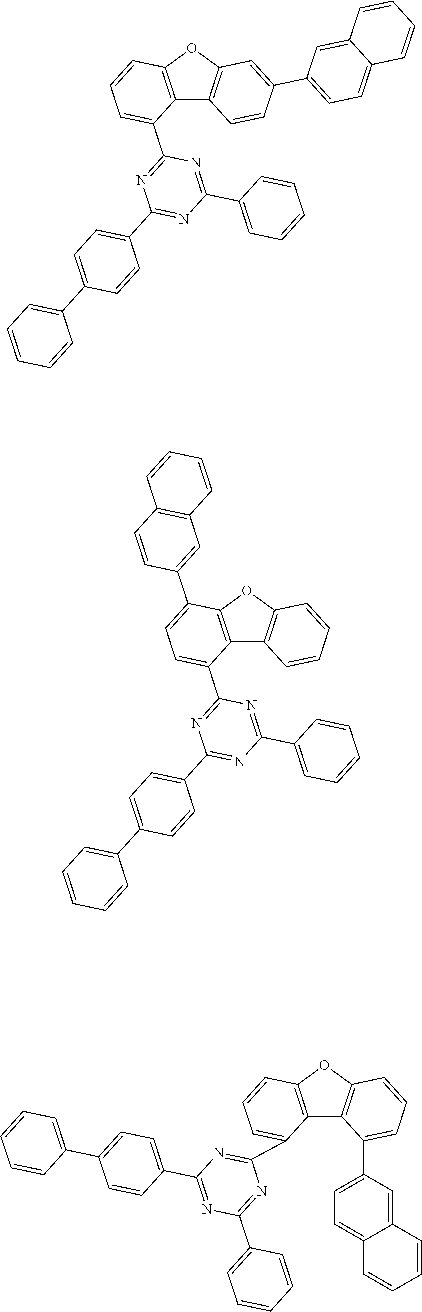 [SMILES: c1ccc(-c2ccc(-c3nc(-c4ccccc4)nc(-c4cccc5oc6cc(-c7ccc8ccccc8c7)ccc6c45)n3)cc2)cc1 ; c1ccc(-c2ccc(-c3nc(-c4ccccc4)nc(-c4ccc(-c5ccc6ccccc6c5)c5oc6ccccc6c45)n3)cc2)cc1 ; c1ccc(-c2ccc(-c3nc(-c4ccccc4)nc(-c4cccc5oc6cccc(-c7ccc8ccccc8c7)c6c45)n3)cc2)cc1]

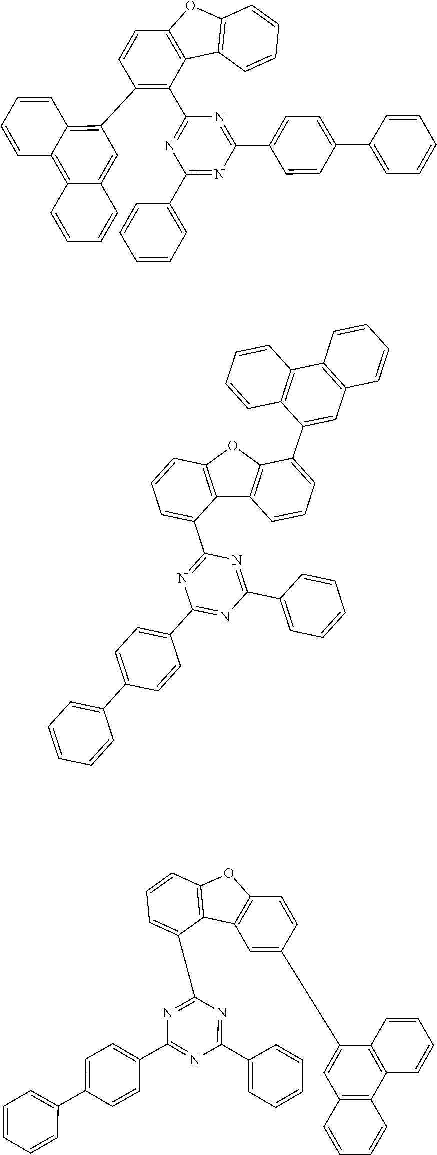 [SMILES: c1ccc(-c2ccc(-c3nc(-c4ccccc4)nc(-c4c(-c5cc6ccccc6c6ccccc56)ccc5oc6ccccc6c45)n3)cc2)cc1 ; c1ccc(-c2ccc(-c3nc(-c4ccccc4)nc(-c4cccc5oc6c(-c7cc8ccccc8c8ccccc78)cccc6c45)n3)cc2)cc1 ; c1ccc(-c2ccc(-c3nc(-c4ccccc4)nc(-c4cccc5oc6ccc(-c7cc8ccccc8c8ccccc78)cc6c45)n3)cc2)cc1]

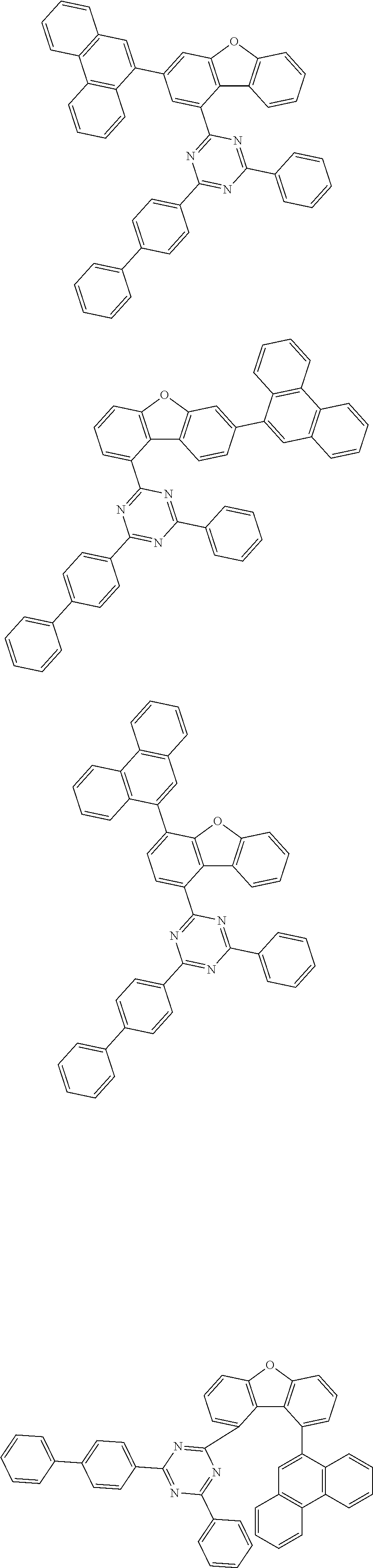 [SMILES: c1ccc(-c2ccc(-c3nc(-c4ccccc4)nc(-c4cc(-c5cc6ccccc6c6ccccc56)cc5oc6ccccc6c45)n3)cc2)cc1 ; c1ccc(-c2ccc(-c3nc(-c4ccccc4)nc(-c4cccc5oc6cc(-c7cc8ccccc8c8ccccc78)ccc6c45)n3)cc2)cc1 ; c1ccc(-c2ccc(-c3nc(-c4ccccc4)nc(-c4ccc(-c5cc6ccccc6c6ccccc56)c5oc6ccccc6c45)n3)cc2)cc1 ; c1ccc(-c2ccc(-c3nc(-c4ccccc4)nc(-c4cccc5oc6cccc(-c7cc8ccccc8c8ccccc78)c6c45)n3)cc2)cc1]

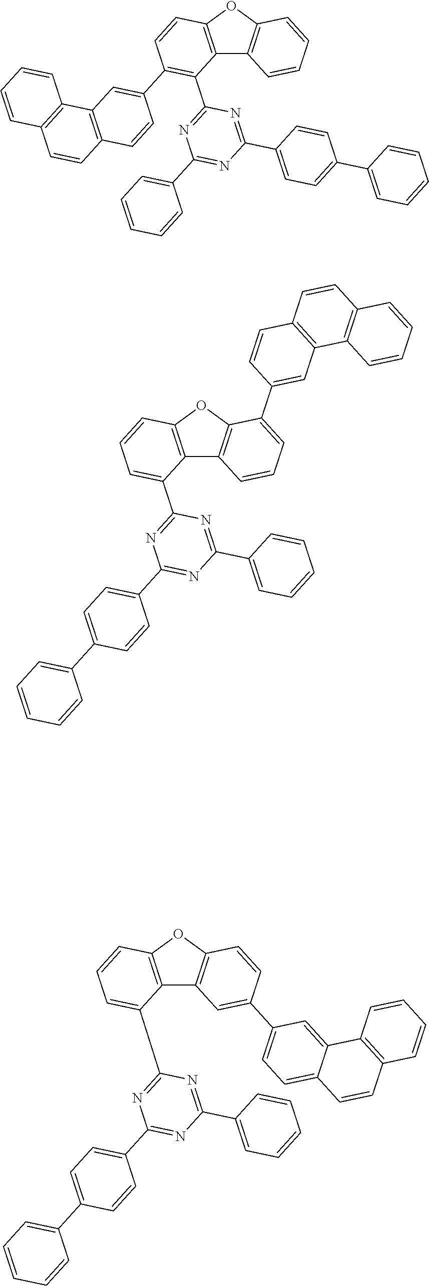 [SMILES: c1ccc(-c2ccc(-c3nc(-c4ccccc4)nc(-c4c(-c5ccc6ccc7ccccc7c6c5)ccc5oc6ccccc6c45)n3)cc2)cc1 ; c1ccc(-c2ccc(-c3nc(-c4ccccc4)nc(-c4cccc5oc6c(-c7ccc8ccc9ccccc9c8c7)cccc6c45)n3)cc2)cc1 ; c1ccc(-c2ccc(-c3nc(-c4ccccc4)nc(-c4cccc5oc6ccc(-c7ccc8ccc9ccccc9c8c7)cc6c45)n3)cc2)cc1]

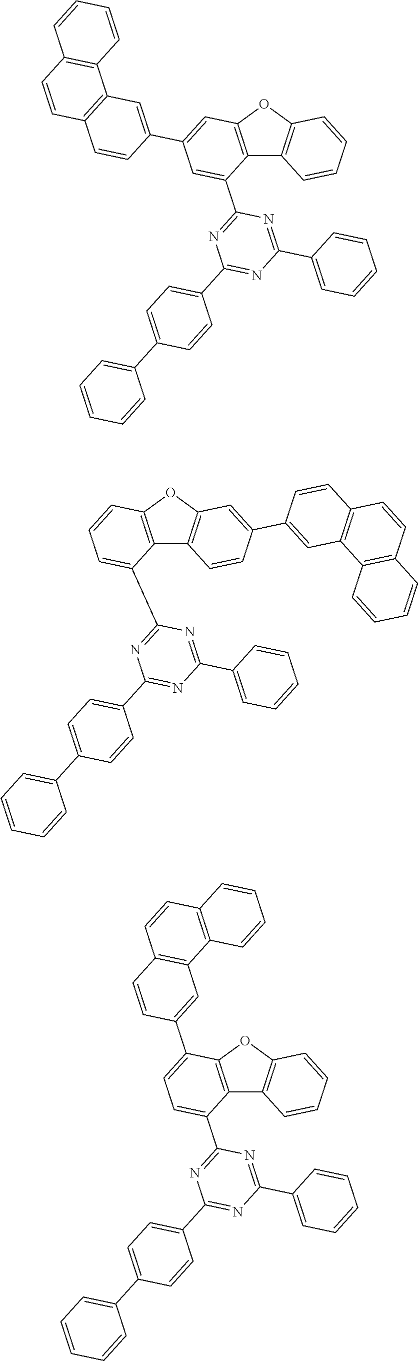 [SMILES: c1ccc(-c2ccc(-c3nc(-c4ccccc4)nc(-c4cc(-c5ccc6ccc7ccccc7c6c5)cc5oc6ccccc6c45)n3)cc2)cc1 ; c1ccc(-c2ccc(-c3nc(-c4ccccc4)nc(-c4cccc5oc6cc(-c7ccc8ccc9ccccc9c8c7)ccc6c45)n3)cc2)cc1 ; c1ccc(-c2ccc(-c3nc(-c4ccccc4)nc(-c4ccc(-c5ccc6ccc7ccccc7c6c5)c5oc6ccccc6c45)n3)cc2)cc1]

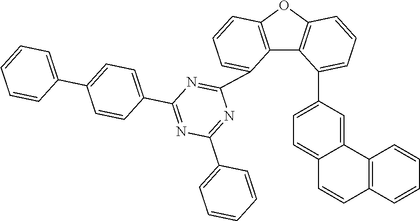 [SMILES: c1ccc(-c2ccc(-c3nc(-c4ccccc4)nc(-c4cccc5oc6cccc(-c7ccc8ccc9ccccc9c8c7)c6c45)n3)cc2)cc1]

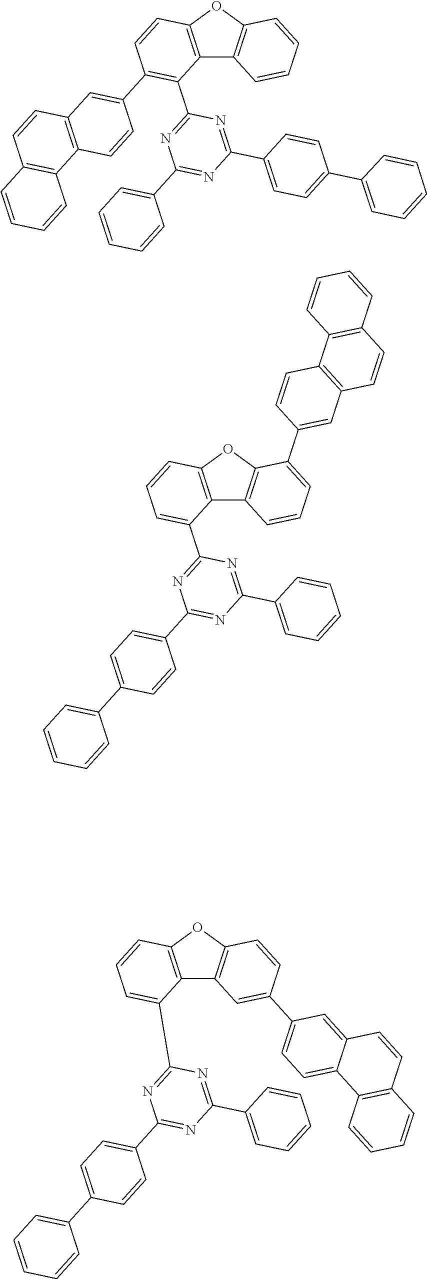 [SMILES: c1ccc(-c2ccc(-c3nc(-c4ccccc4)nc(-c4c(-c5ccc6c(ccc7ccccc76)c5)ccc5oc6ccccc6c45)n3)cc2)cc1 ; c1ccc(-c2ccc(-c3nc(-c4ccccc4)nc(-c4cccc5oc6c(-c7ccc8c(ccc9ccccc98)c7)cccc6c45)n3)cc2)cc1 ; c1ccc(-c2ccc(-c3nc(-c4ccccc4)nc(-c4cccc5oc6ccc(-c7ccc8c(ccc9ccccc98)c7)cc6c45)n3)cc2)cc1]

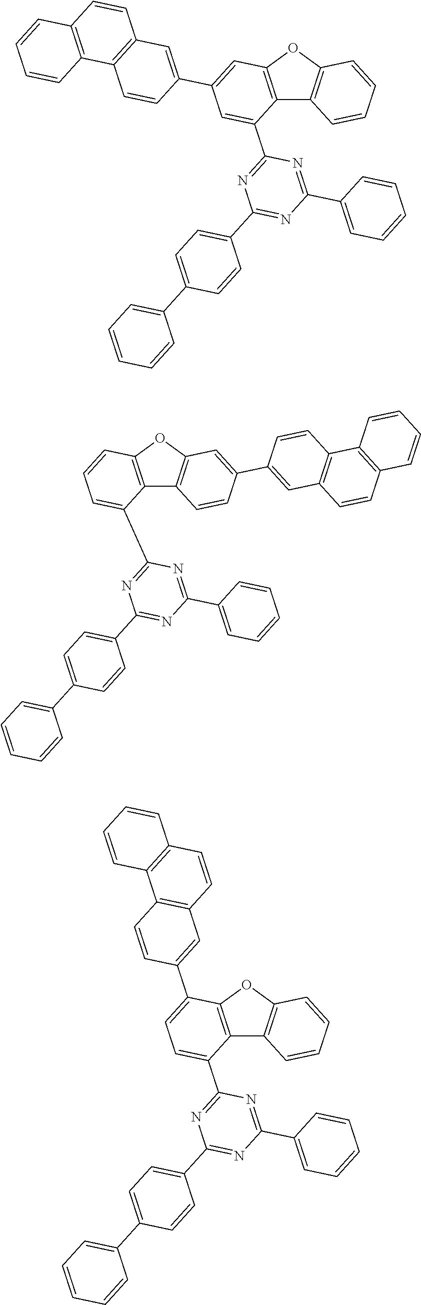 [SMILES: c1ccc(-c2ccc(-c3nc(-c4ccccc4)nc(-c4cc(-c5ccc6c(ccc7ccccc76)c5)cc5oc6ccccc6c45)n3)cc2)cc1 ; c1ccc(-c2ccc(-c3nc(-c4ccccc4)nc(-c4cccc5oc6cc(-c7ccc8c(ccc9ccccc98)c7)ccc6c45)n3)cc2)cc1 ; c1ccc(-c2ccc(-c3nc(-c4ccccc4)nc(-c4ccc(-c5ccc6c(ccc7ccccc76)c5)c5oc6ccccc6c45)n3)cc2)cc1]

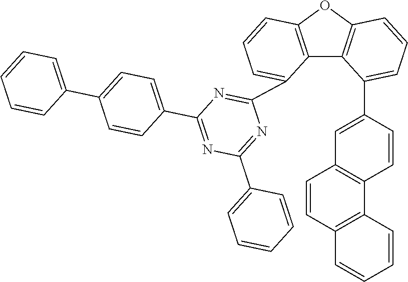 [SMILES: c1ccc(-c2ccc(-c3nc(-c4ccccc4)nc(-c4cccc5oc6cccc(-c7ccc8c(ccc9ccccc98)c7)c6c45)n3)cc2)cc1]

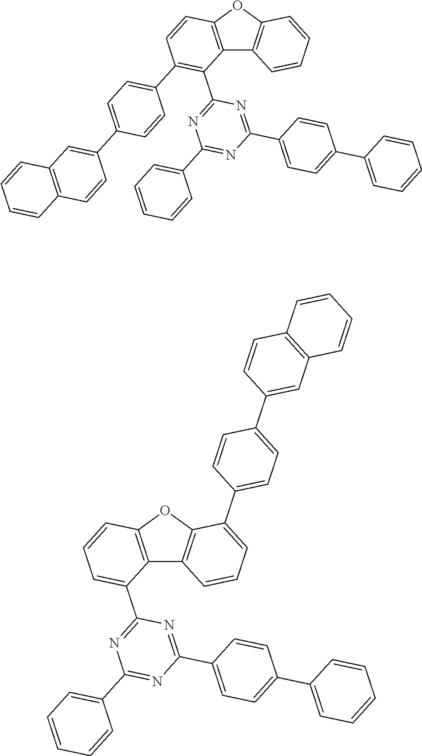 [SMILES: c1ccc(-c2ccc(-c3nc(-c4ccccc4)nc(-c4c(-c5ccc(-c6ccc7ccccc7c6)cc5)ccc5oc6ccccc6c45)n3)cc2)cc1 ; c1ccc(-c2ccc(-c3nc(-c4ccccc4)nc(-c4cccc5oc6c(-c7ccc(-c8ccc9ccccc9c8)cc7)cccc6c45)n3)cc2)cc1]

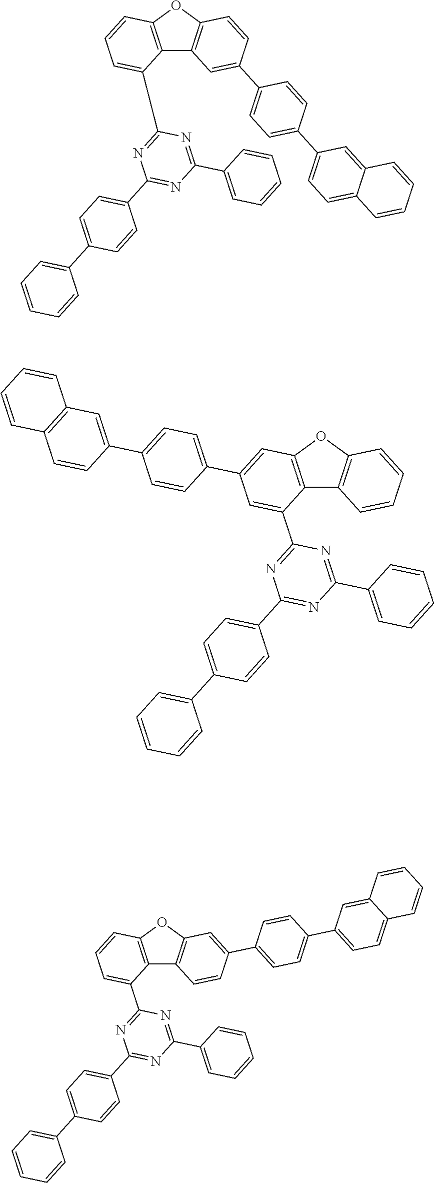 [SMILES: c1ccc(-c2ccc(-c3nc(-c4ccccc4)nc(-c4cccc5oc6ccc(-c7ccc(-c8ccc9ccccc9c8)cc7)cc6c45)n3)cc2)cc1 ; c1ccc(-c2ccc(-c3nc(-c4ccccc4)nc(-c4cc(-c5ccc(-c6ccc7ccccc7c6)cc5)cc5oc6ccccc6c45)n3)cc2)cc1 ; c1ccc(-c2ccc(-c3nc(-c4ccccc4)nc(-c4cccc5oc6cc(-c7ccc(-c8ccc9ccccc9c8)cc7)ccc6c45)n3)cc2)cc1]

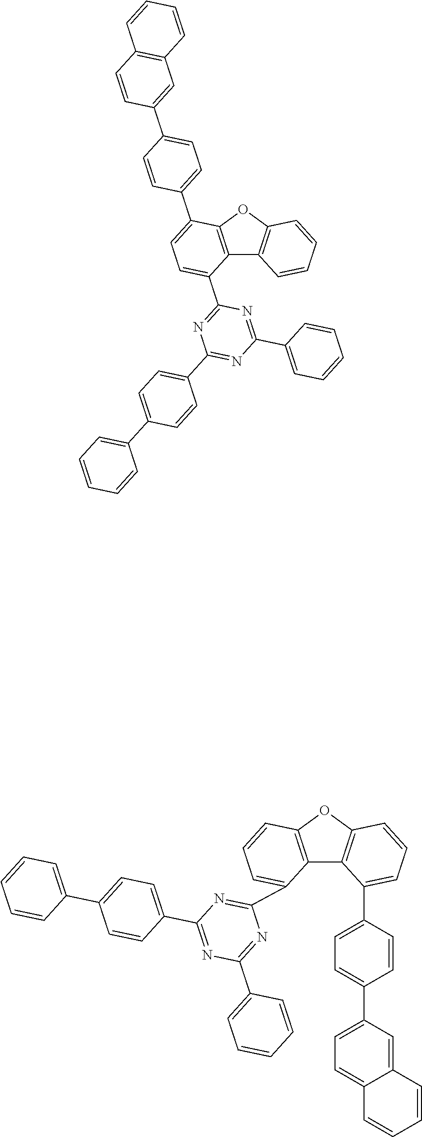 [SMILES: c1ccc(-c2ccc(-c3nc(-c4ccccc4)nc(-c4ccc(-c5ccc(-c6ccc7ccccc7c6)cc5)c5oc6ccccc6c45)n3)cc2)cc1 ; c1ccc(-c2ccc(-c3nc(-c4ccccc4)nc(-c4cccc5oc6cccc(-c7ccc(-c8ccc9ccccc9c8)cc7)c6c45)n3)cc2)cc1]

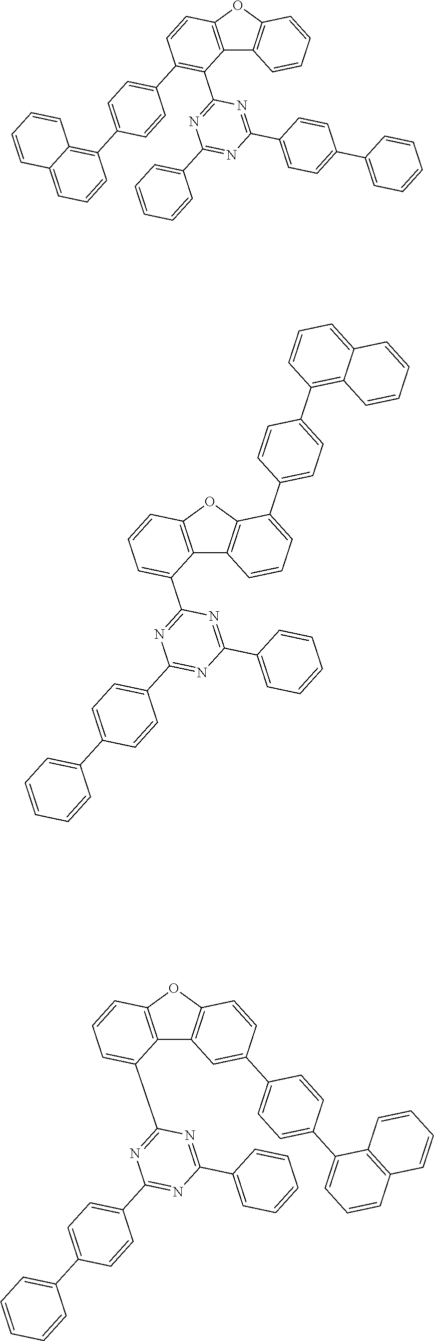 [SMILES: c1ccc(-c2ccc(-c3nc(-c4ccccc4)nc(-c4c(-c5ccc(-c6cccc7ccccc67)cc5)ccc5oc6ccccc6c45)n3)cc2)cc1 ; c1ccc(-c2ccc(-c3nc(-c4ccccc4)nc(-c4cccc5oc6c(-c7ccc(-c8cccc9ccccc89)cc7)cccc6c45)n3)cc2)cc1 ; c1ccc(-c2ccc(-c3nc(-c4ccccc4)nc(-c4cccc5oc6ccc(-c7ccc(-c8cccc9ccccc89)cc7)cc6c45)n3)cc2)cc1]

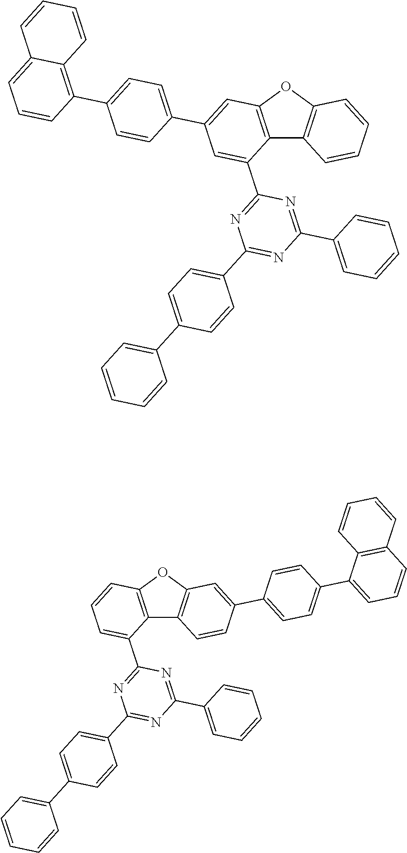 [SMILES: c1ccc(-c2ccc(-c3nc(-c4ccccc4)nc(-c4cc(-c5ccc(-c6cccc7ccccc67)cc5)cc5oc6ccccc6c45)n3)cc2)cc1 ; c1ccc(-c2ccc(-c3nc(-c4ccccc4)nc(-c4cccc5oc6cc(-c7ccc(-c8cccc9ccccc89)cc7)ccc6c45)n3)cc2)cc1]

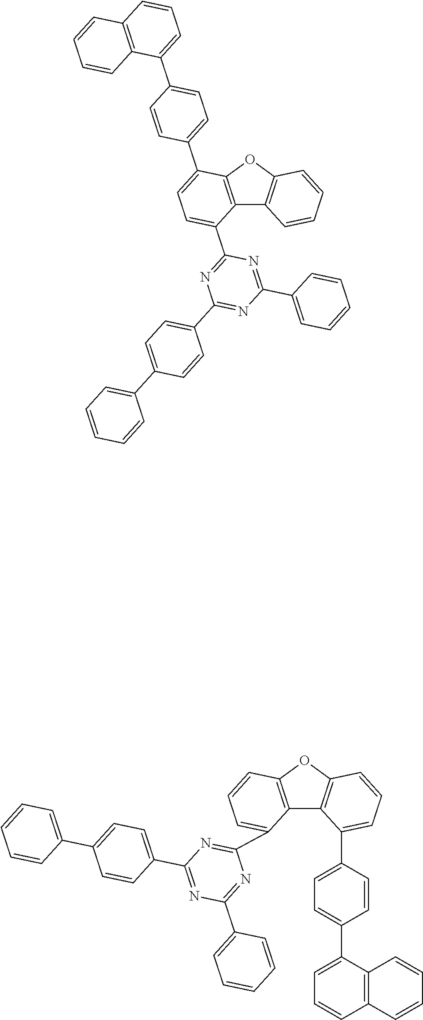 [SMILES: c1ccc(-c2ccc(-c3nc(-c4ccccc4)nc(-c4ccc(-c5ccc(-c6cccc7ccccc67)cc5)c5oc6ccccc6c45)n3)cc2)cc1 ; c1ccc(-c2ccc(-c3nc(-c4ccccc4)nc(-c4cccc5oc6cccc(-c7ccc(-c8cccc9ccccc89)cc7)c6c45)n3)cc2)cc1]

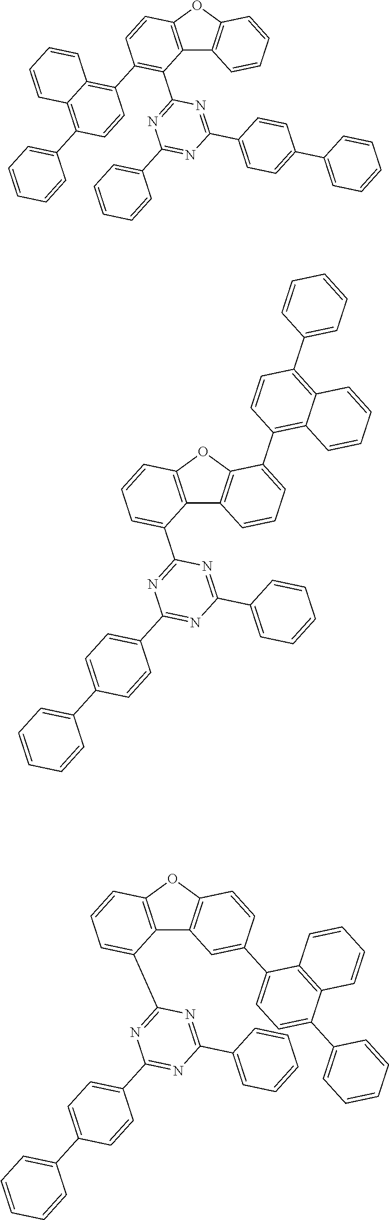 [SMILES: c1ccc(-c2ccc(-c3nc(-c4ccccc4)nc(-c4c(-c5ccc(-c6ccccc6)c6ccccc56)ccc5oc6ccccc6c45)n3)cc2)cc1 ; c1ccc(-c2ccc(-c3nc(-c4ccccc4)nc(-c4cccc5oc6c(-c7ccc(-c8ccccc8)c8ccccc78)cccc6c45)n3)cc2)cc1 ; c1ccc(-c2ccc(-c3nc(-c4ccccc4)nc(-c4cccc5oc6ccc(-c7ccc(-c8ccccc8)c8ccccc78)cc6c45)n3)cc2)cc1]

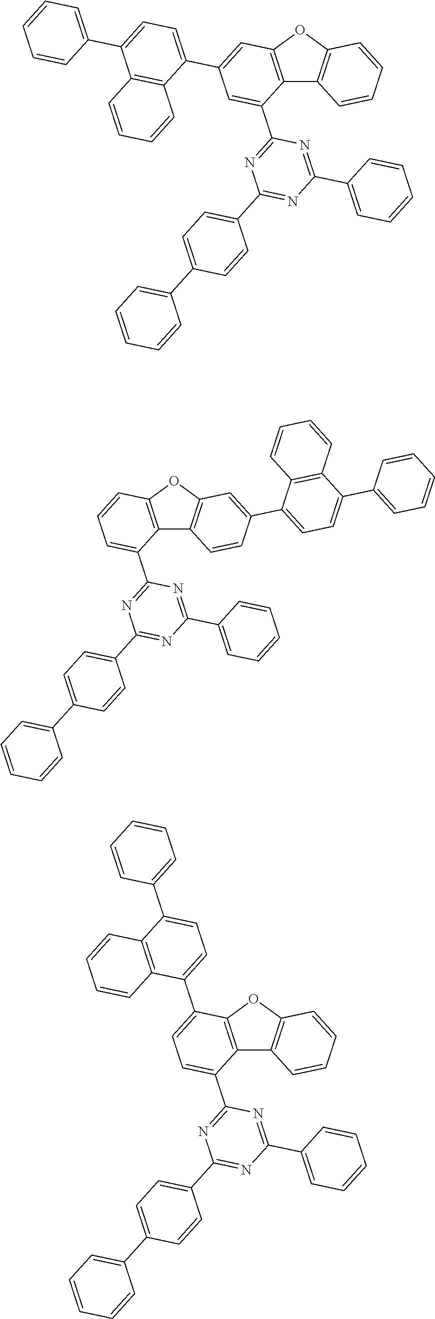 [SMILES: c1ccc(-c2ccc(-c3nc(-c4ccccc4)nc(-c4cc(-c5ccc(-c6ccccc6)c6ccccc56)cc5oc6ccccc6c45)n3)cc2)cc1 ; c1ccc(-c2ccc(-c3nc(-c4ccccc4)nc(-c4cccc5oc6cc(-c7ccc(-c8ccccc8)c8ccccc78)ccc6c45)n3)cc2)cc1 ; c1ccc(-c2ccc(-c3nc(-c4ccccc4)nc(-c4ccc(-c5ccc(-c6ccccc6)c6ccccc56)c5oc6ccccc6c45)n3)cc2)cc1]

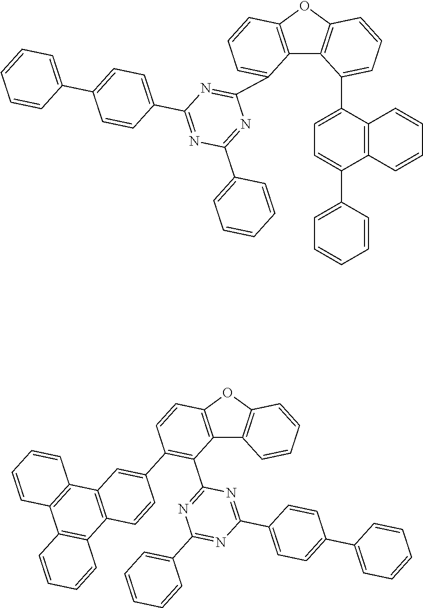 [SMILES: c1ccc(-c2ccc(-c3nc(-c4ccccc4)nc(-c4cccc5oc6cccc(-c7ccc(-c8ccccc8)c8ccccc78)c6c45)n3)cc2)cc1 ; c1ccc(-c2ccc(-c3nc(-c4ccccc4)nc(-c4c(-c5ccc6c7ccccc7c7ccccc7c6c5)ccc5oc6ccccc6c45)n3)cc2)cc1]

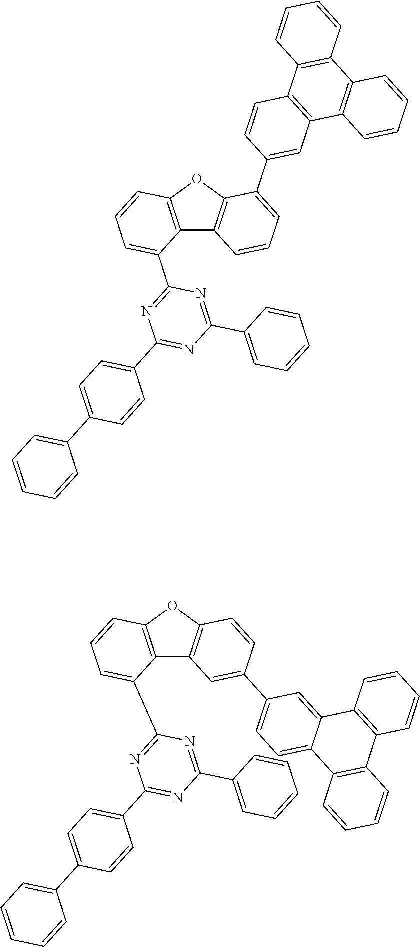 [SMILES: c1ccc(-c2ccc(-c3nc(-c4ccccc4)nc(-c4cccc5oc6c(-c7ccc8c9ccccc9c9ccccc9c8c7)cccc6c45)n3)cc2)cc1 ; c1ccc(-c2ccc(-c3nc(-c4ccccc4)nc(-c4cccc5oc6ccc(-c7ccc8c9ccccc9c9ccccc9c8c7)cc6c45)n3)cc2)cc1]

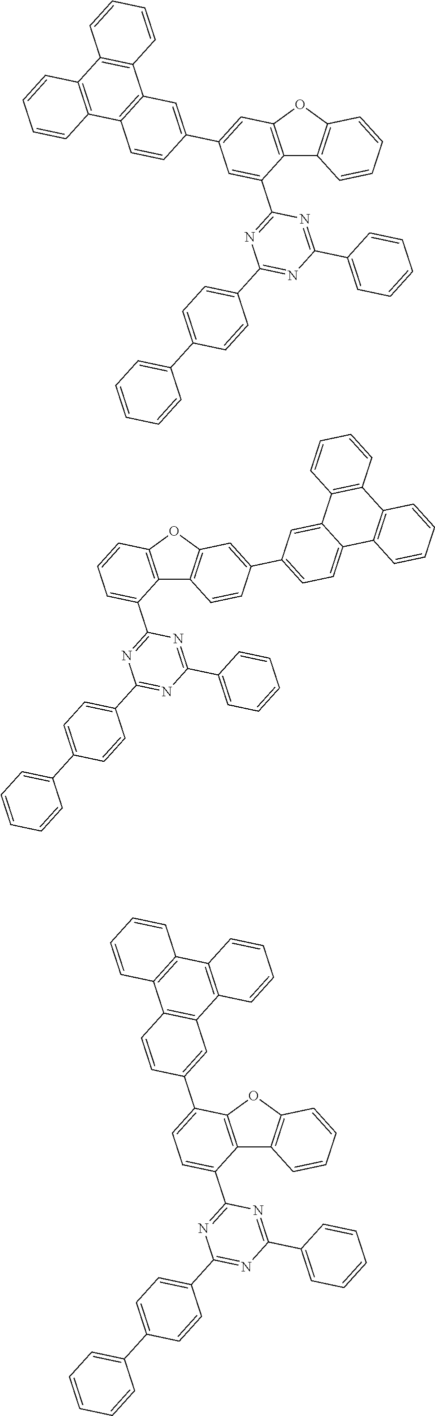 [SMILES: c1ccc(-c2ccc(-c3nc(-c4ccccc4)nc(-c4cc(-c5ccc6c7ccccc7c7ccccc7c6c5)cc5oc6ccccc6c45)n3)cc2)cc1 ; c1ccc(-c2ccc(-c3nc(-c4ccccc4)nc(-c4cccc5oc6cc(-c7ccc8c9ccccc9c9ccccc9c8c7)ccc6c45)n3)cc2)cc1 ; c1ccc(-c2ccc(-c3nc(-c4ccccc4)nc(-c4ccc(-c5ccc6c7ccccc7c7ccccc7c6c5)c5oc6ccccc6c45)n3)cc2)cc1]

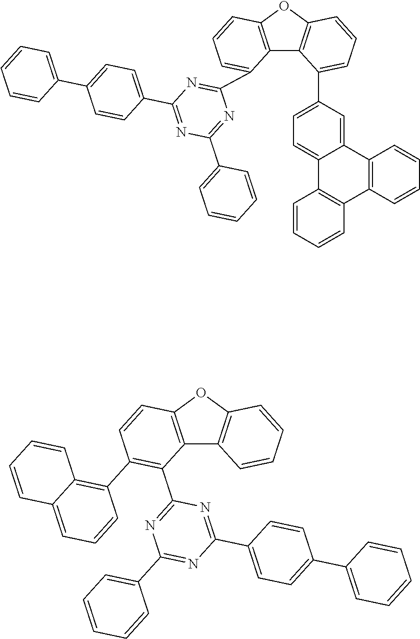 [SMILES: c1ccc(-c2ccc(-c3nc(-c4ccccc4)nc(-c4cccc5oc6cccc(-c7ccc8c9ccccc9c9ccccc9c8c7)c6c45)n3)cc2)cc1 ; c1ccc(-c2ccc(-c3nc(-c4ccccc4)nc(-c4c(-c5cccc6ccccc56)ccc5oc6ccccc6c45)n3)cc2)cc1]

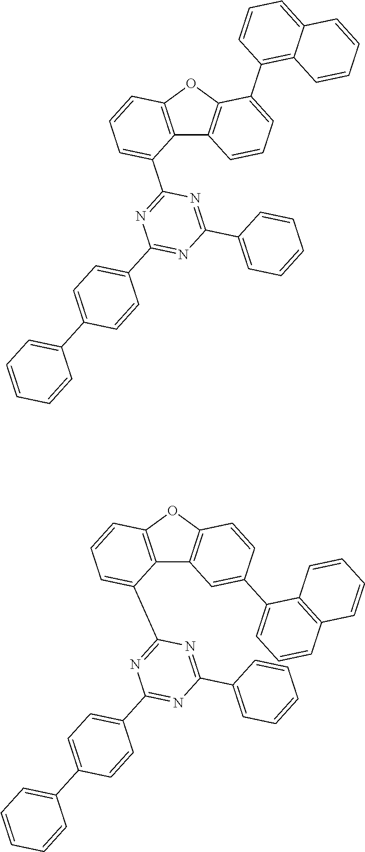 [SMILES: c1ccc(-c2ccc(-c3nc(-c4ccccc4)nc(-c4cccc5oc6c(-c7cccc8ccccc78)cccc6c45)n3)cc2)cc1 ; c1ccc(-c2ccc(-c3nc(-c4ccccc4)nc(-c4cccc5oc6ccc(-c7cccc8ccccc78)cc6c45)n3)cc2)cc1]

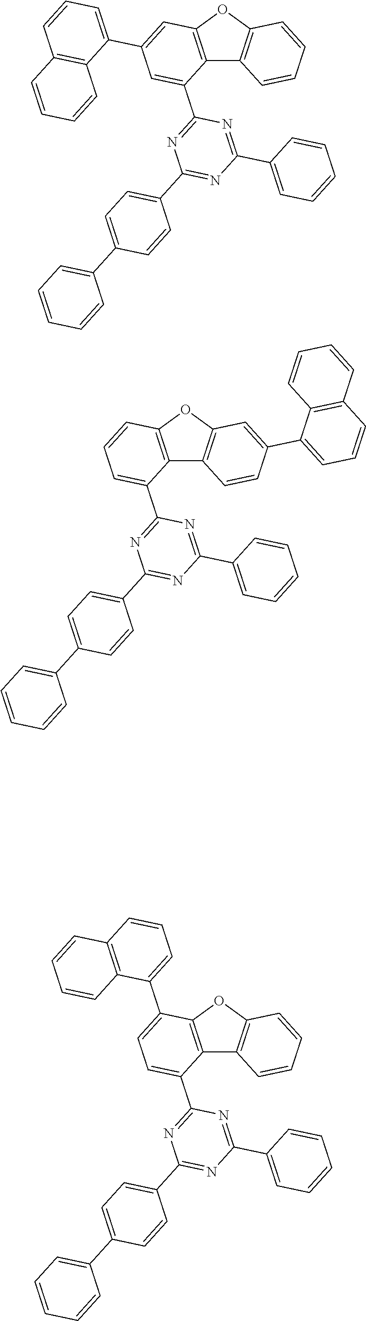 [SMILES: c1ccc(-c2ccc(-c3nc(-c4ccccc4)nc(-c4cc(-c5cccc6ccccc56)cc5oc6ccccc6c45)n3)cc2)cc1 ; c1ccc(-c2ccc(-c3nc(-c4ccccc4)nc(-c4cccc5oc6cc(-c7cccc8ccccc78)ccc6c45)n3)cc2)cc1 ; c1ccc(-c2ccc(-c3nc(-c4ccccc4)nc(-c4ccc(-c5cccc6ccccc56)c5oc6ccccc6c45)n3)cc2)cc1]

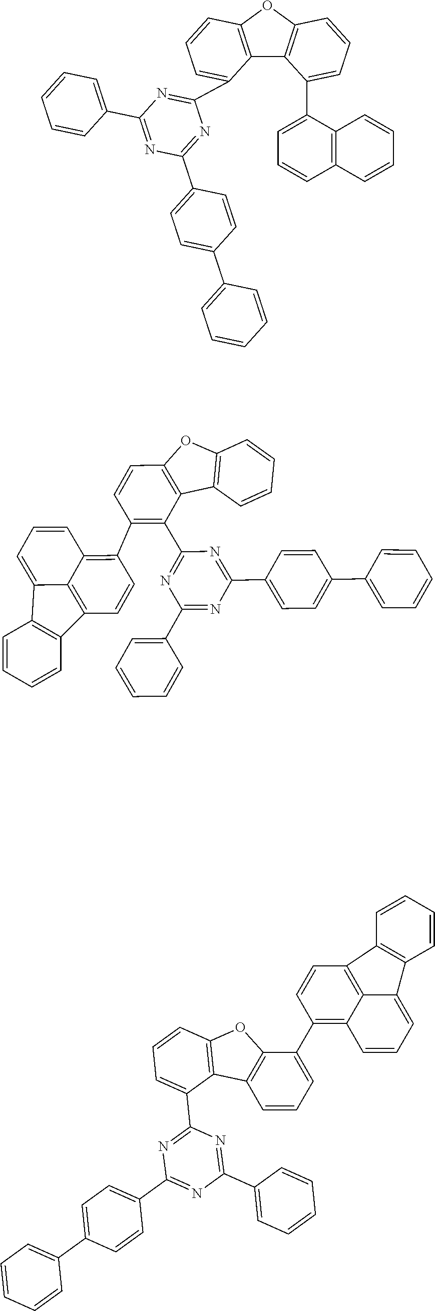 [SMILES: c1ccc(-c2ccc(-c3nc(-c4ccccc4)nc(-c4cccc5oc6cccc(-c7cccc8ccccc78)c6c45)n3)cc2)cc1 ; c1ccc(-c2ccc(-c3nc(-c4ccccc4)nc(-c4c(-c5ccc6c7c(cccc57)-c5ccccc5-6)ccc5oc6ccccc6c45)n3)cc2)cc1 ; c1ccc(-c2ccc(-c3nc(-c4ccccc4)nc(-c4cccc5oc6c(-c7ccc8c9c(cccc79)-c7ccccc7-8)cccc6c45)n3)cc2)cc1]

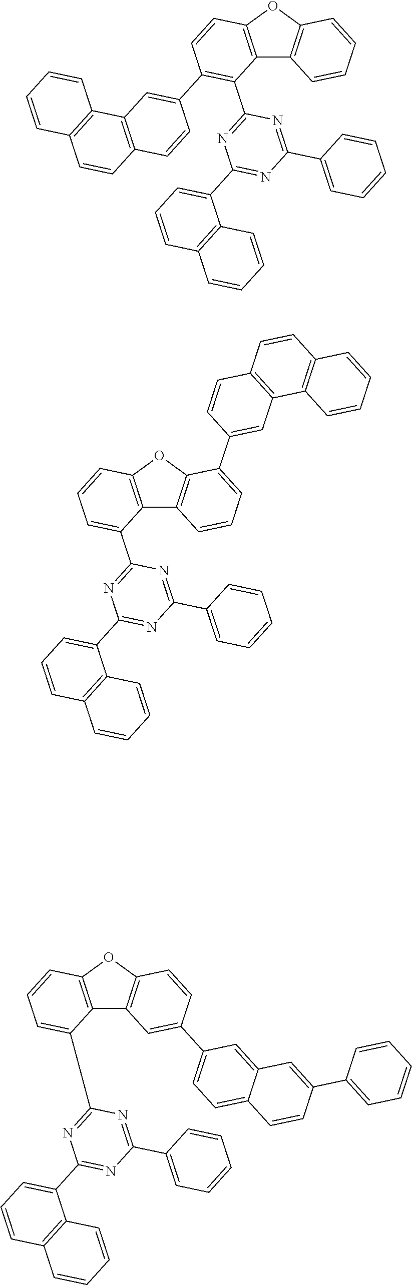 [SMILES: c1ccc(-c2nc(-c3cccc4ccccc34)nc(-c3c(-c4ccc5ccc6ccccc6c5c4)ccc4oc5ccccc5c34)n2)cc1 ; c1ccc(-c2nc(-c3cccc4ccccc34)nc(-c3cccc4oc5c(-c6ccc7ccc8ccccc8c7c6)cccc5c34)n2)cc1 ; c1ccc(-c2ccc3ccc(-c4ccc5oc6cccc(-c7nc(-c8ccccc8)nc(-c8cccc9ccccc89)n7)c6c5c4)cc3c2)cc1]

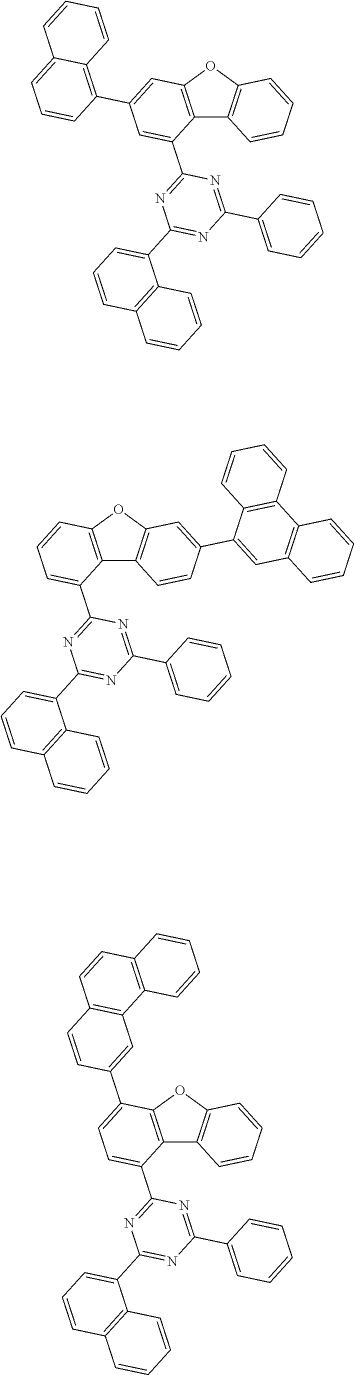 [SMILES: c1ccc(-c2nc(-c3cccc4ccccc34)nc(-c3cc(-c4cccc5ccccc45)cc4oc5ccccc5c34)n2)cc1 ; c1ccc(-c2nc(-c3cccc4ccccc34)nc(-c3cccc4oc5cc(-c6cc7ccccc7c7ccccc67)ccc5c34)n2)cc1 ; c1ccc(-c2nc(-c3cccc4ccccc34)nc(-c3ccc(-c4ccc5ccc6ccccc6c5c4)c4oc5ccccc5c34)n2)cc1]

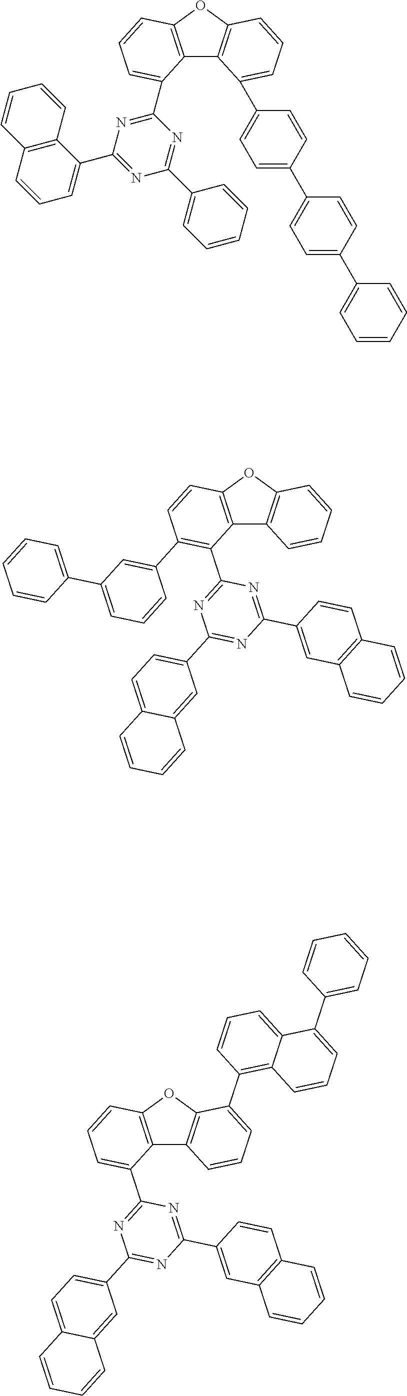 [SMILES: c1ccc(-c2ccc(-c3ccc(-c4cccc5oc6cccc(-c7nc(-c8ccccc8)nc(-c8cccc9ccccc89)n7)c6c45)cc3)cc2)cc1 ; c1ccc(-c2cccc(-c3ccc4oc5ccccc5c4c3-c3nc(-c4ccc5ccccc5c4)nc(-c4ccc5ccccc5c4)n3)c2)cc1 ; c1ccc(-c2cccc3c(-c4cccc5c4oc4cccc(-c6nc(-c7ccc8ccccc8c7)nc(-c7ccc8ccccc8c7)n6)c45)cccc23)cc1]

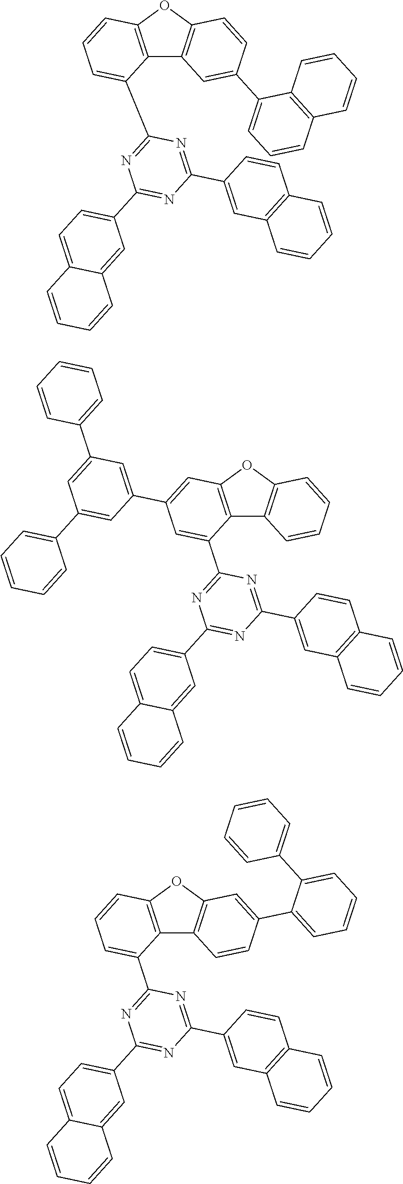

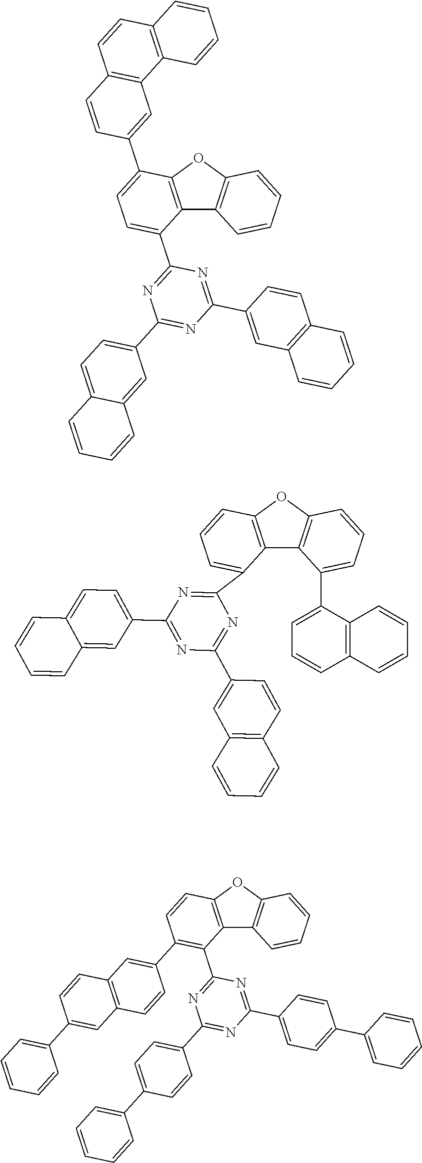 [SMILES: c1ccc2cc(-c3nc(-c4ccc5ccccc5c4)nc(-c4ccc(-c5ccc6ccc7ccccc7c6c5)c5oc6ccccc6c45)n3)ccc2c1 ; c1ccc2cc(-c3nc(-c4ccc5ccccc5c4)nc(-c4cccc5oc6cccc(-c7cccc8ccccc78)c6c45)n3)ccc2c1 ; c1ccc(-c2ccc(-c3nc(-c4ccc(-c5ccccc5)cc4)nc(-c4c(-c5ccc6cc(-c7ccccc7)ccc6c5)ccc5oc6ccccc6c45)n3)cc2)cc1]

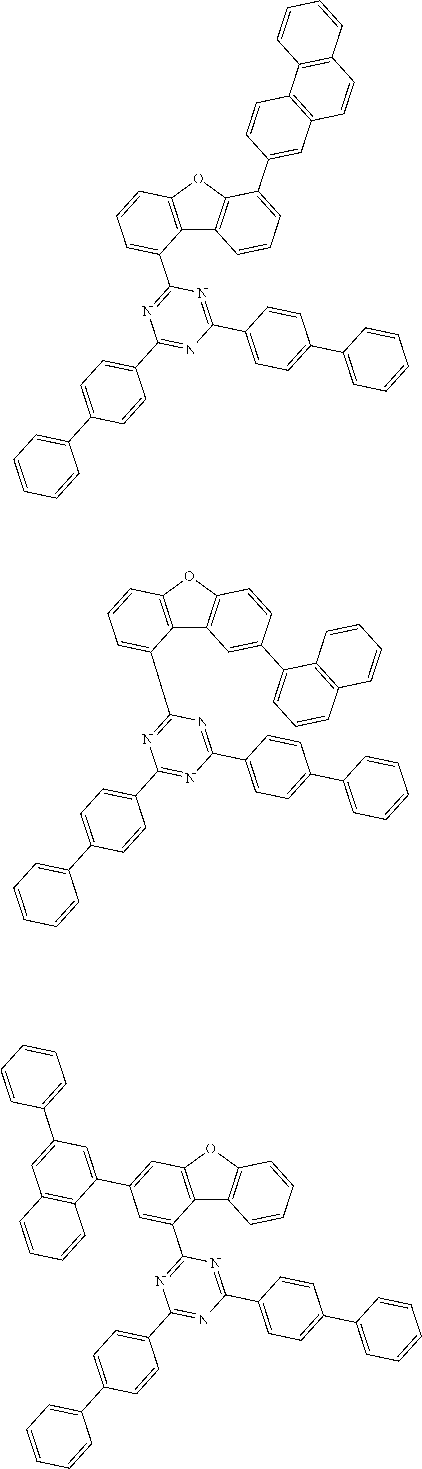 [SMILES: c1ccc(-c2ccc(-c3nc(-c4ccc(-c5ccccc5)cc4)nc(-c4cccc5oc6c(-c7ccc8c(ccc9ccccc98)c7)cccc6c45)n3)cc2)cc1 ; c1ccc(-c2ccc(-c3nc(-c4ccc(-c5ccccc5)cc4)nc(-c4cccc5oc6ccc(-c7cccc8ccccc78)cc6c45)n3)cc2)cc1 ; c1ccc(-c2ccc(-c3nc(-c4ccc(-c5ccccc5)cc4)nc(-c4cc(-c5cc(-c6ccccc6)cc6ccccc56)cc5oc6ccccc6c45)n3)cc2)cc1]

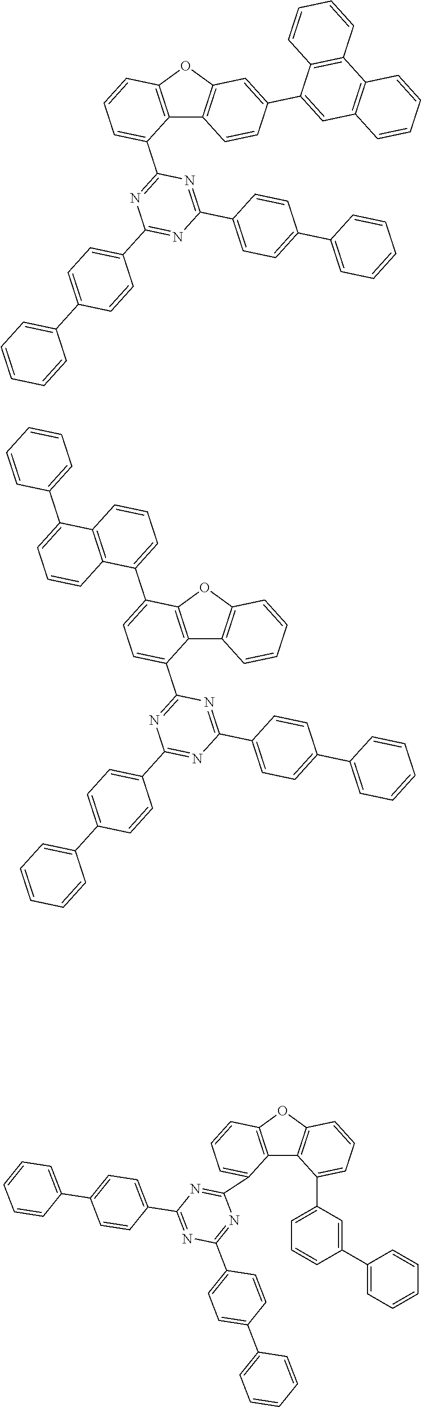

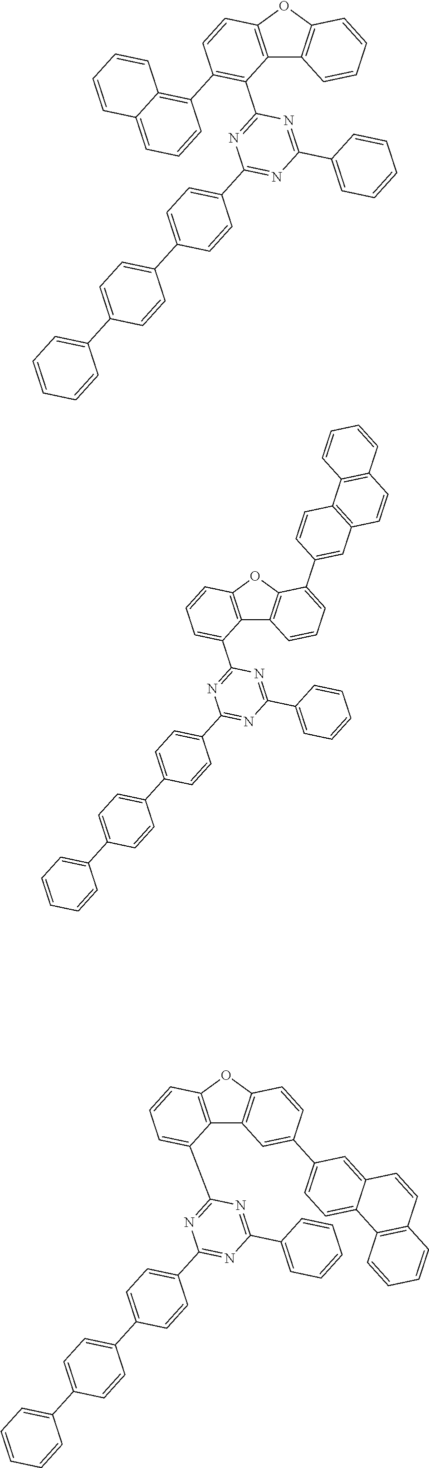 [SMILES: c1ccc(-c2ccc(-c3ccc(-c4nc(-c5ccccc5)nc(-c5c(-c6cccc7ccccc67)ccc6oc7ccccc7c56)n4)cc3)cc2)cc1 ; c1ccc(-c2ccc(-c3ccc(-c4nc(-c5ccccc5)nc(-c5cccc6oc7c(-c8ccc9c(ccc%10ccccc%109)c8)cccc7c56)n4)cc3)cc2)cc1 ; c1ccc(-c2ccc(-c3ccc(-c4nc(-c5ccccc5)nc(-c5cccc6oc7ccc(-c8ccc9c(ccc%10ccccc%109)c8)cc7c56)n4)cc3)cc2)cc1]

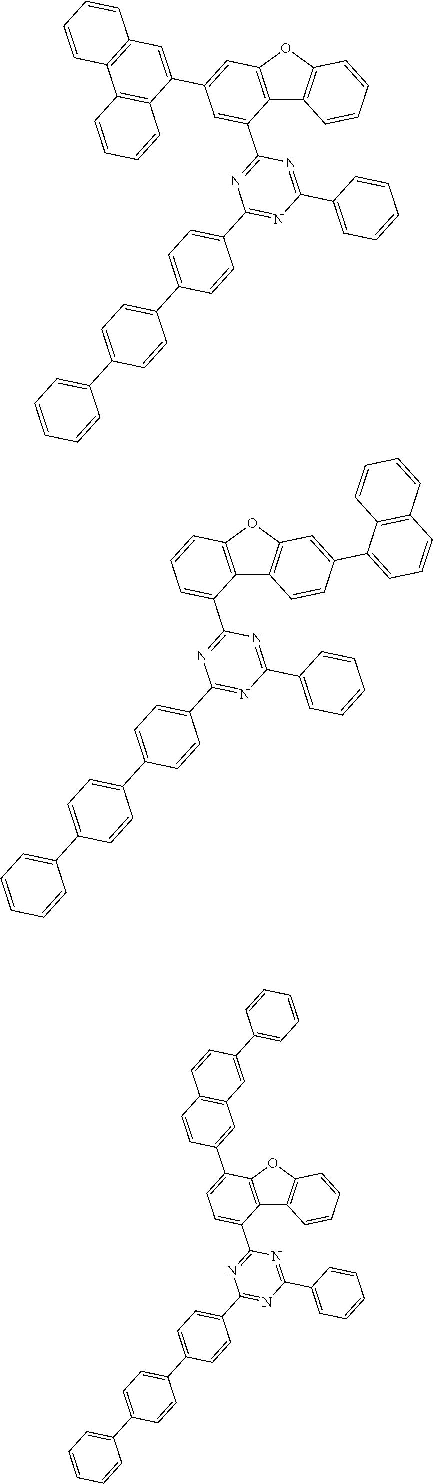 [SMILES: c1ccc(-c2ccc(-c3ccc(-c4nc(-c5ccccc5)nc(-c5cc(-c6cc7ccccc7c7ccccc67)cc6oc7ccccc7c56)n4)cc3)cc2)cc1 ; c1ccc(-c2ccc(-c3ccc(-c4nc(-c5ccccc5)nc(-c5cccc6oc7cc(-c8cccc9ccccc89)ccc7c56)n4)cc3)cc2)cc1 ; c1ccc(-c2ccc(-c3ccc(-c4nc(-c5ccccc5)nc(-c5ccc(-c6ccc7ccc(-c8ccccc8)cc7c6)c6oc7ccccc7c56)n4)cc3)cc2)cc1]

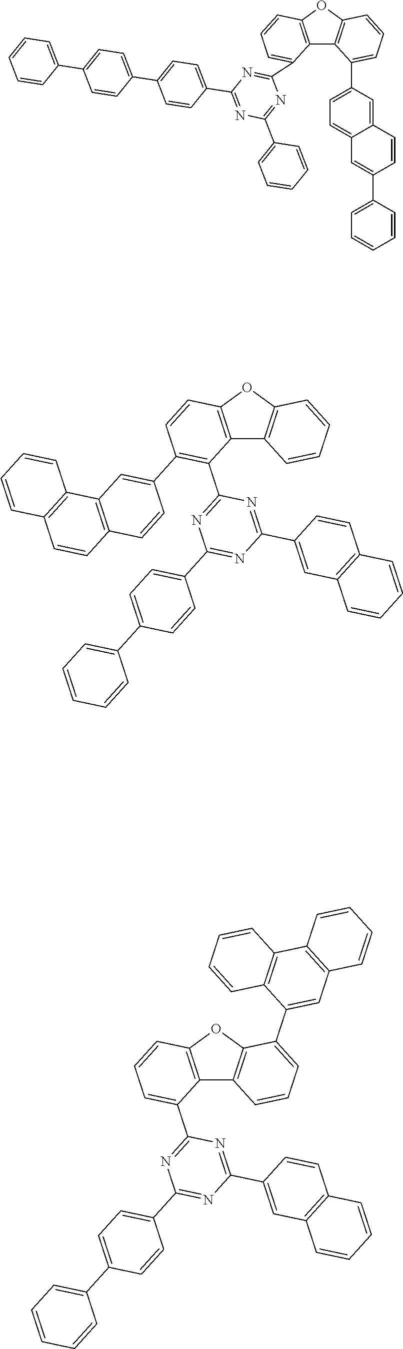 [SMILES: c1ccc(-c2ccc(-c3ccc(-c4nc(-c5ccccc5)nc(-c5cccc6oc7cccc(-c8ccc9cc(-c%10ccccc%10)ccc9c8)c7c56)n4)cc3)cc2)cc1 ; c1ccc(-c2ccc(-c3nc(-c4ccc5ccccc5c4)nc(-c4c(-c5ccc6ccc7ccccc7c6c5)ccc5oc6ccccc6c45)n3)cc2)cc1 ; c1ccc(-c2ccc(-c3nc(-c4ccc5ccccc5c4)nc(-c4cccc5oc6c(-c7cc8ccccc8c8ccccc78)cccc6c45)n3)cc2)cc1]

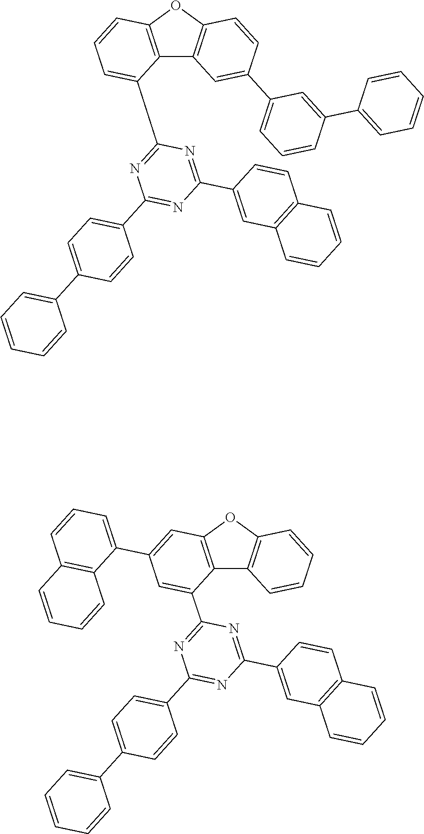 [SMILES: c1ccc(-c2ccc(-c3nc(-c4ccc5ccccc5c4)nc(-c4cccc5oc6ccc(-c7cccc(-c8ccccc8)c7)cc6c45)n3)cc2)cc1 ; c1ccc(-c2ccc(-c3nc(-c4ccc5ccccc5c4)nc(-c4cc(-c5cccc6ccccc56)cc5oc6ccccc6c45)n3)cc2)cc1]

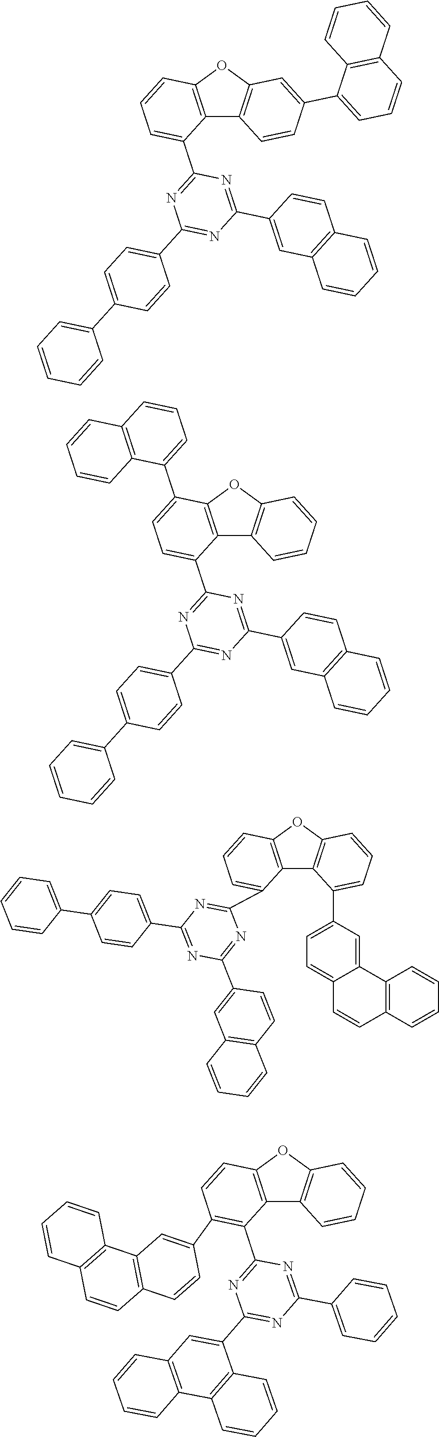

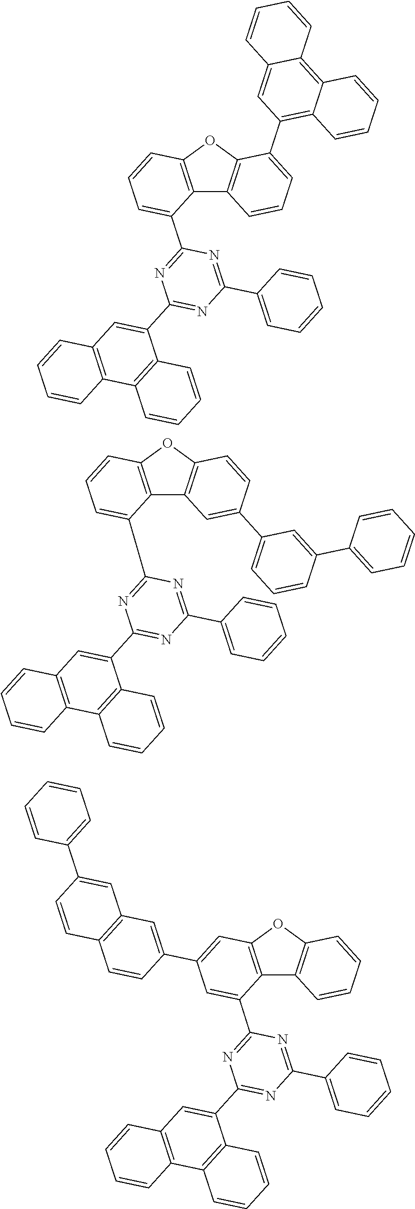

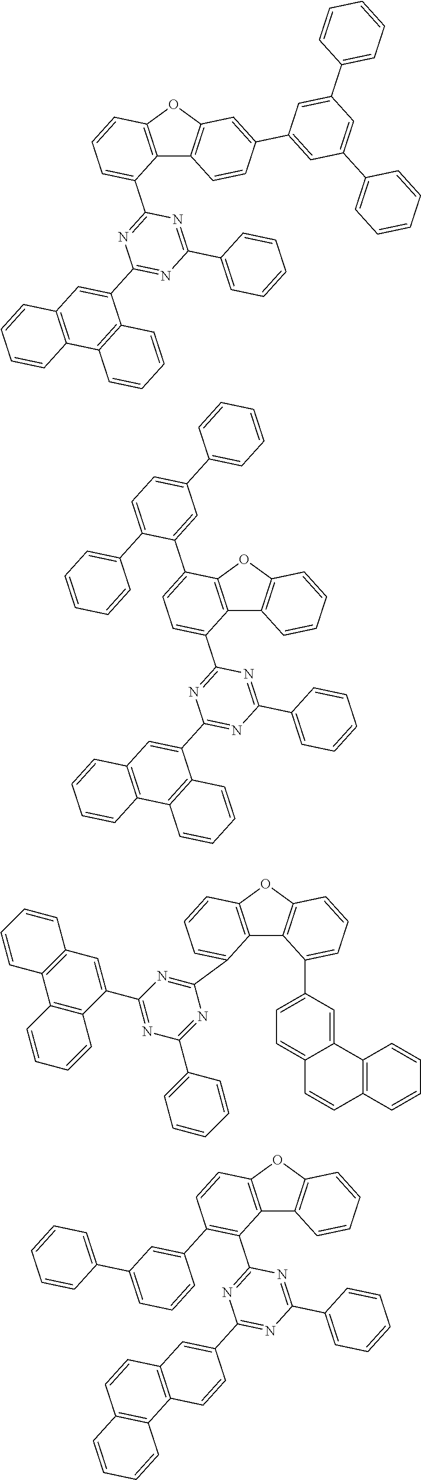

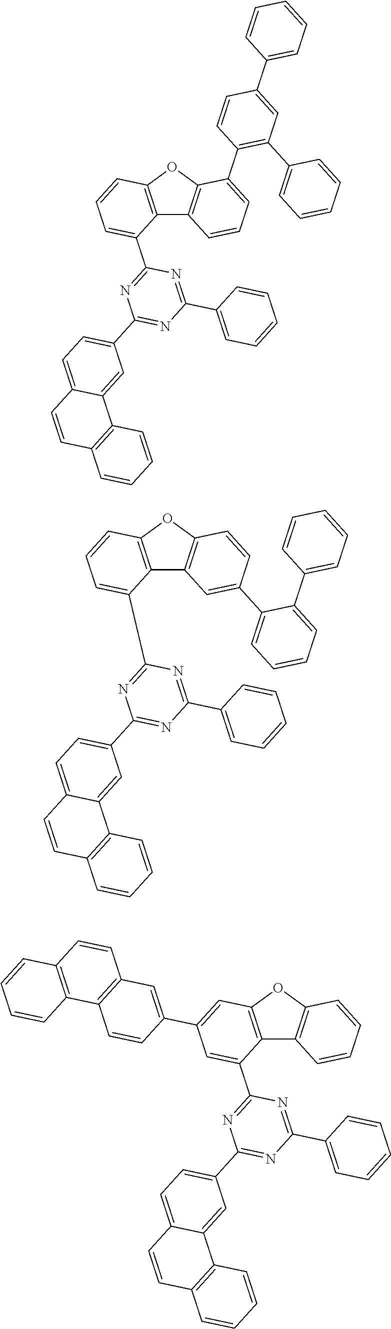

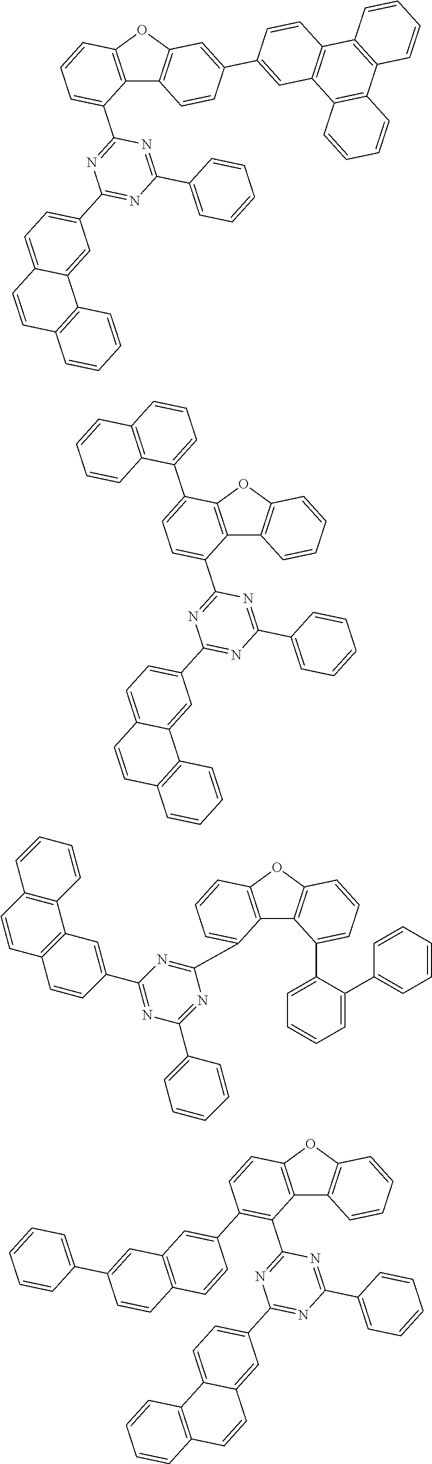

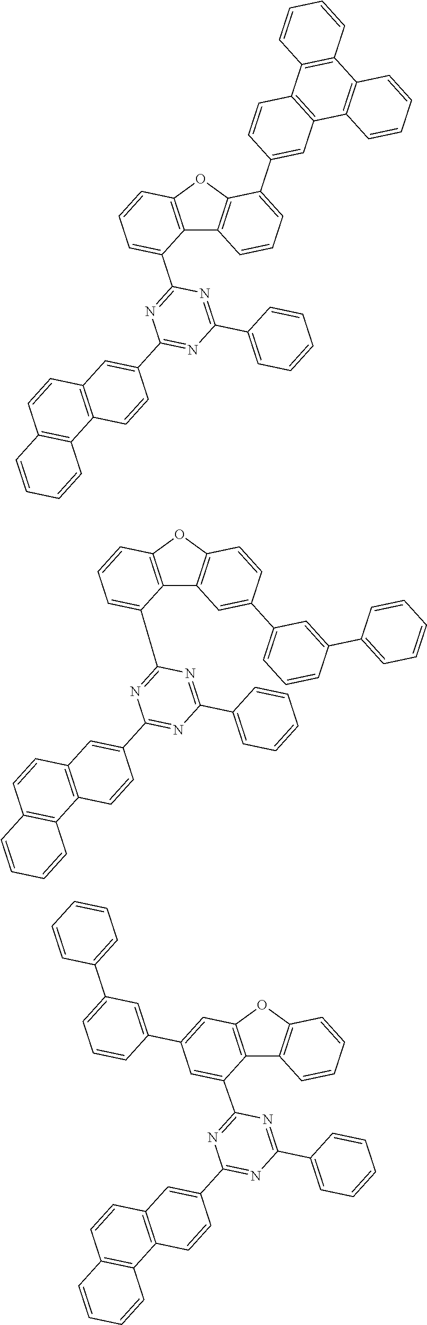

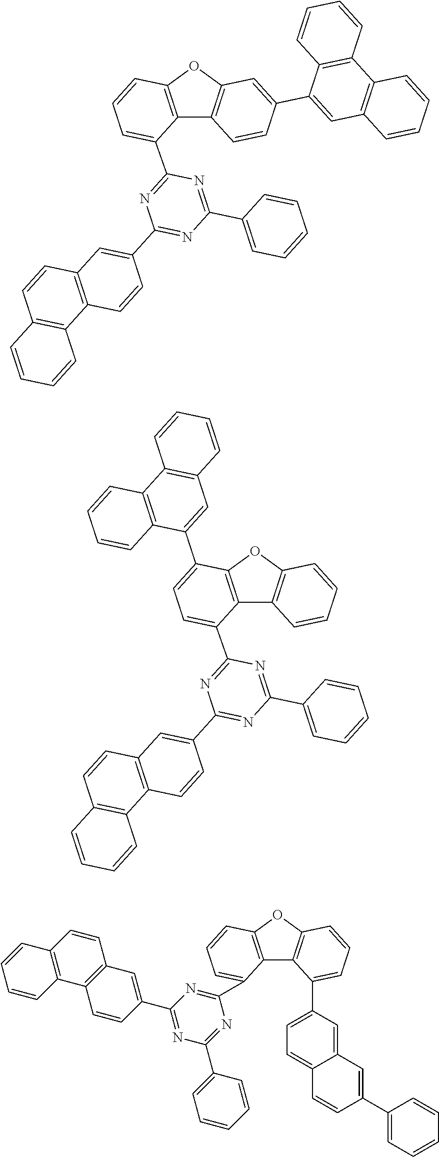

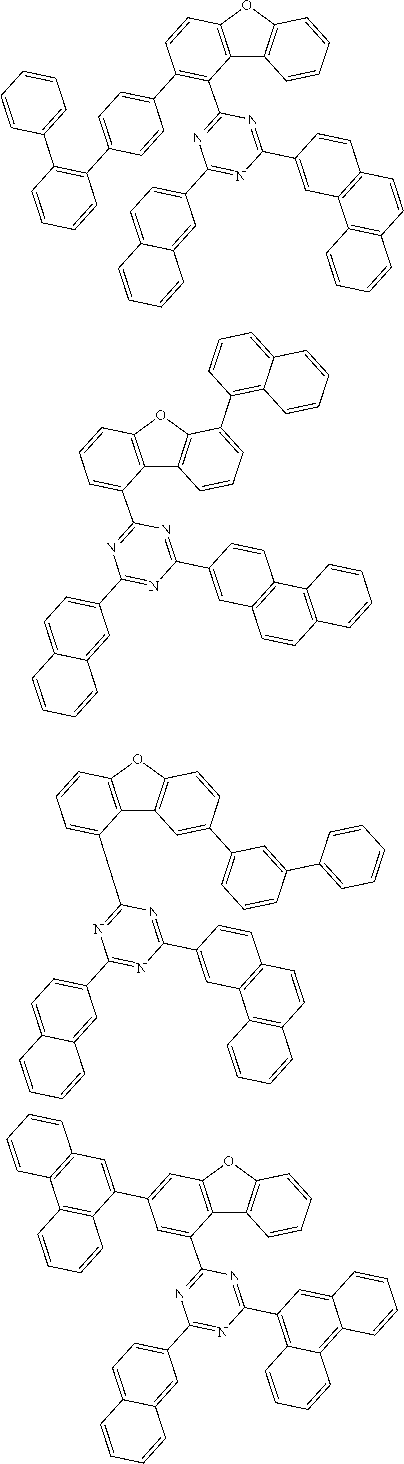

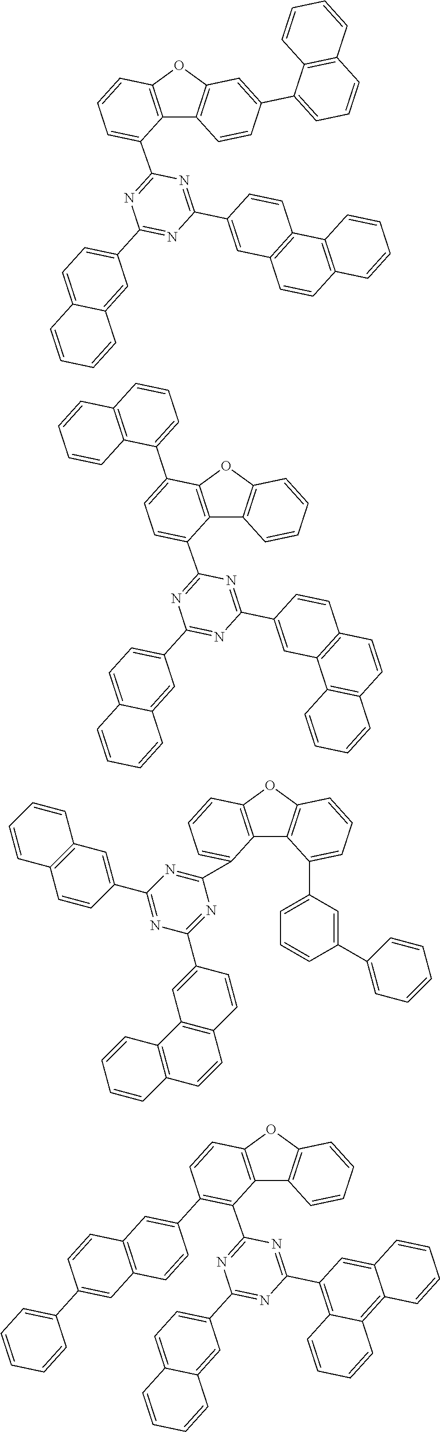

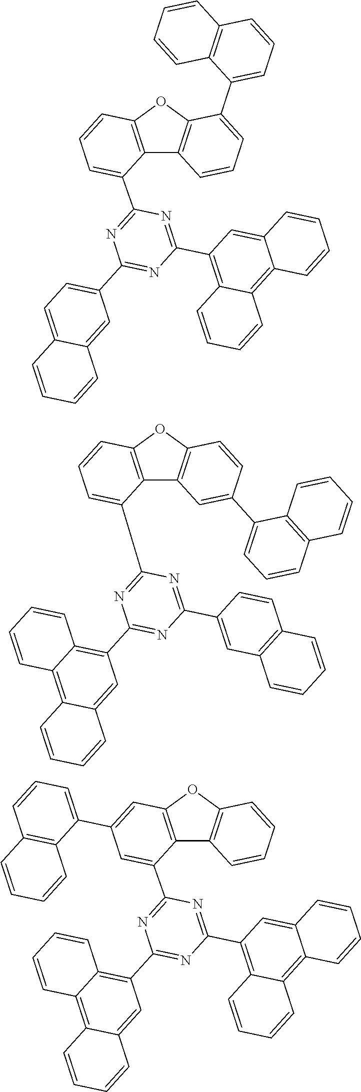

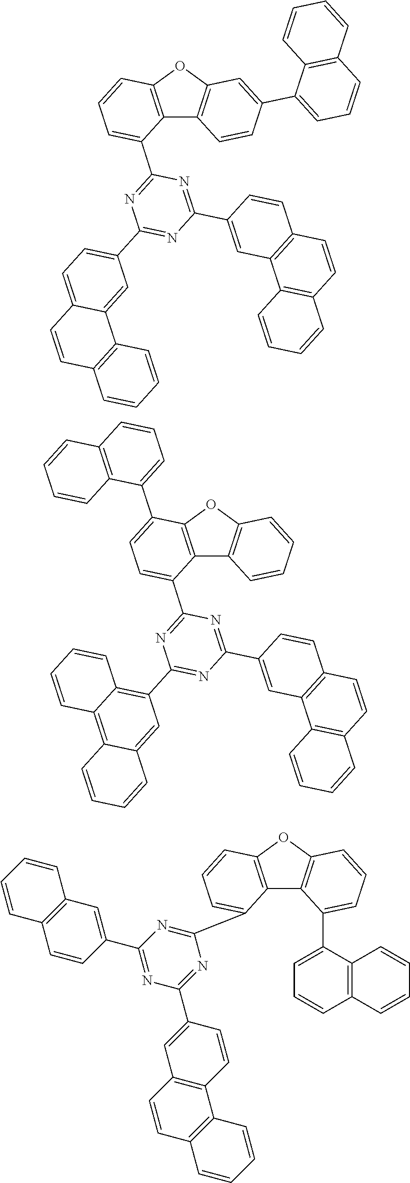

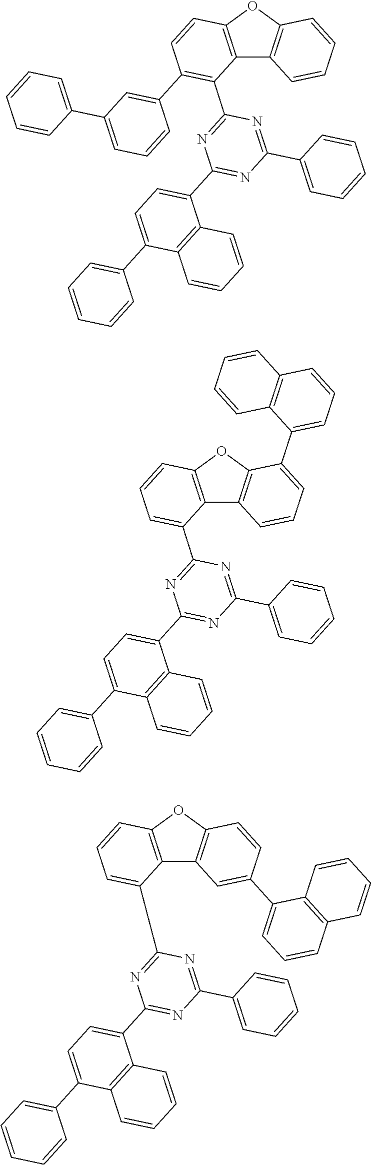

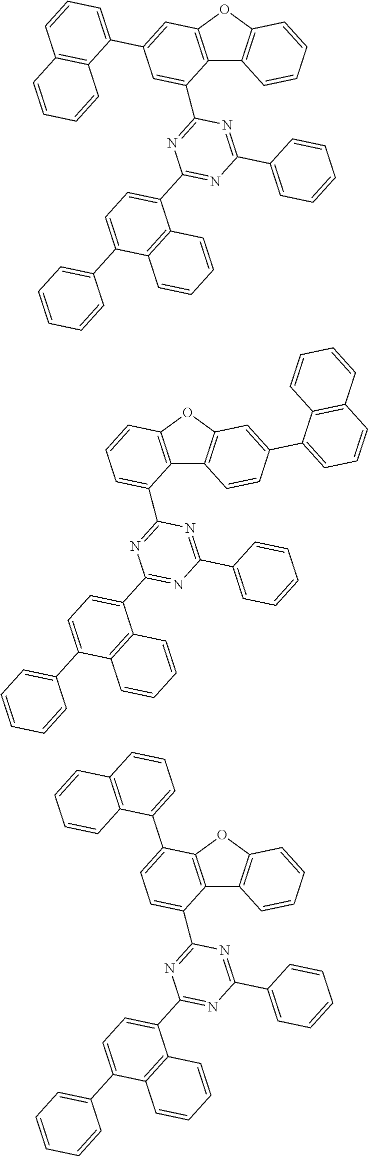

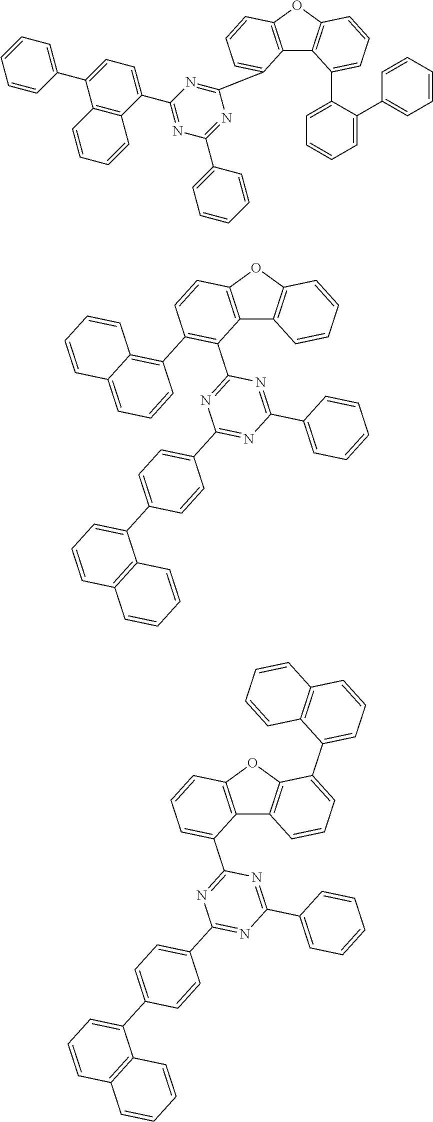

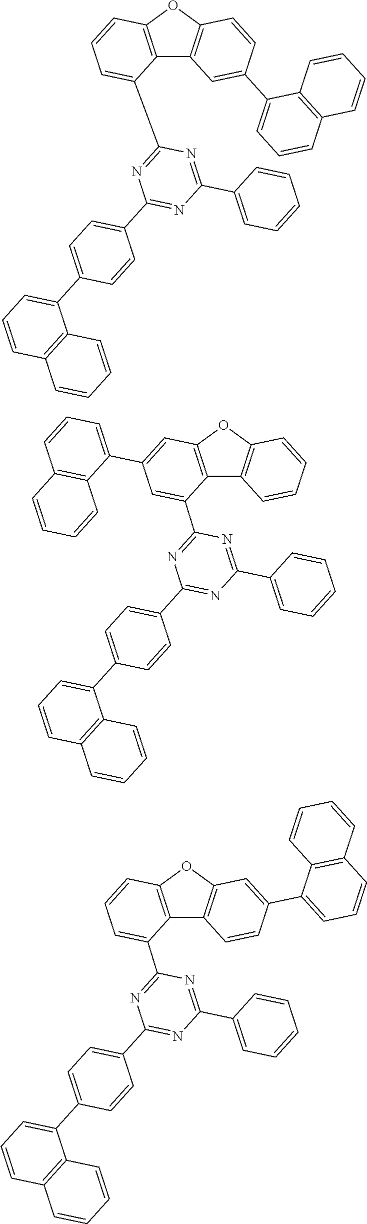

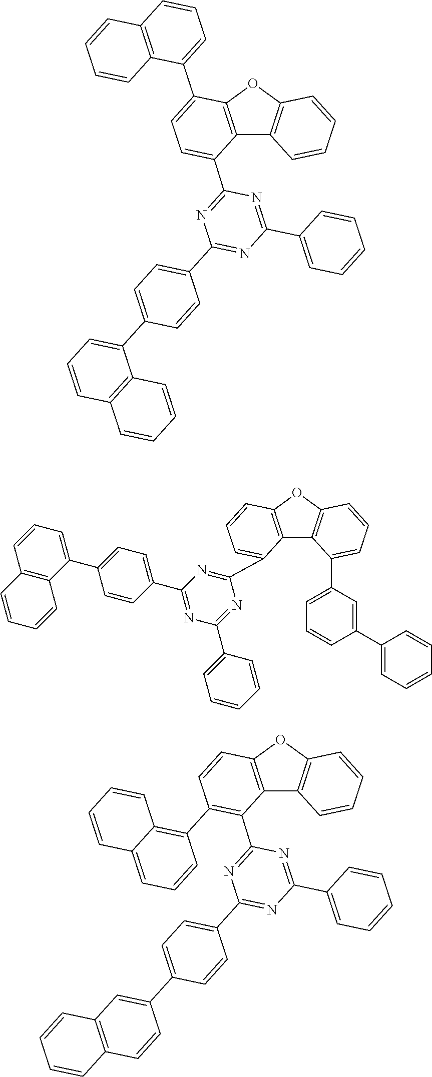

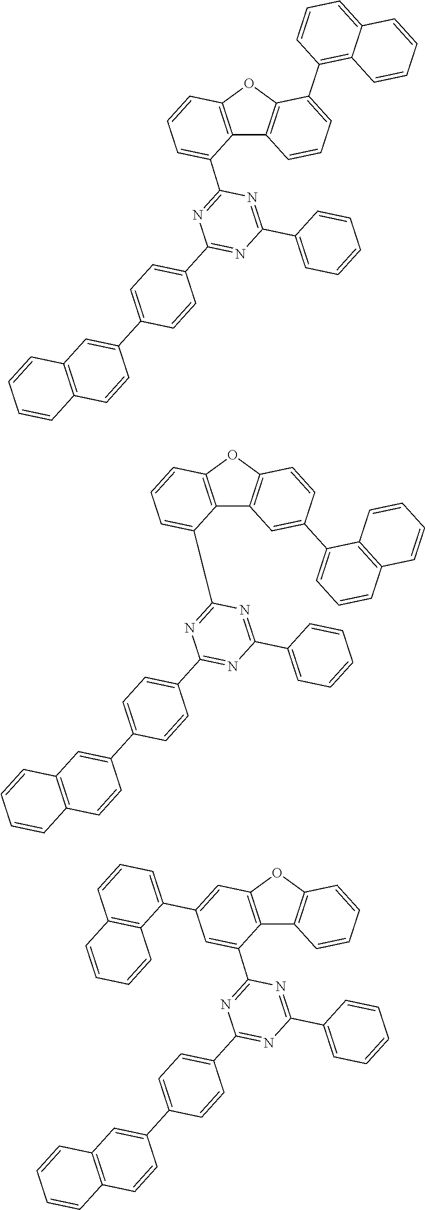

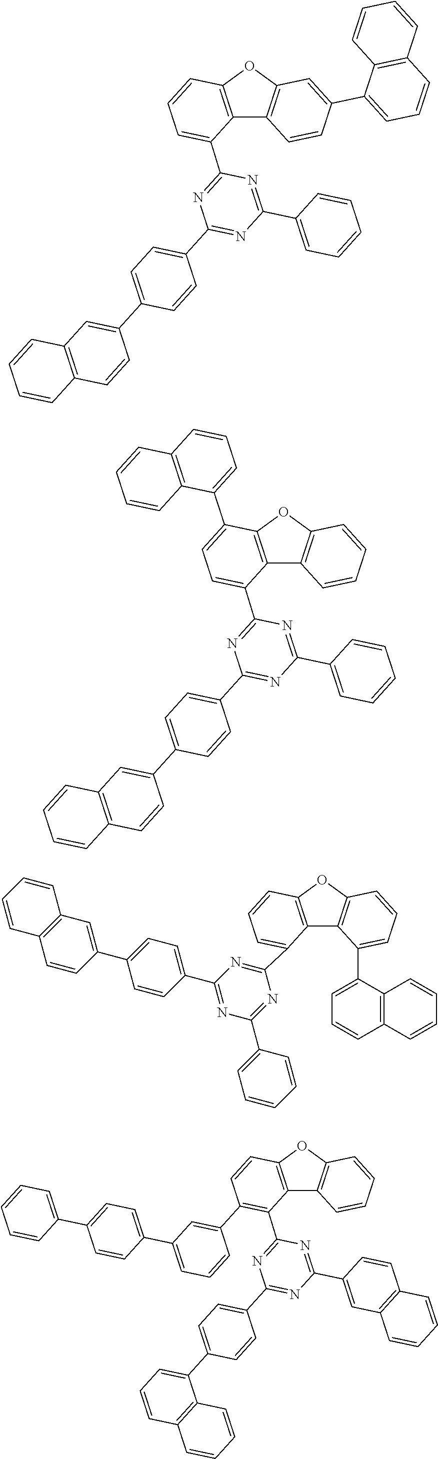

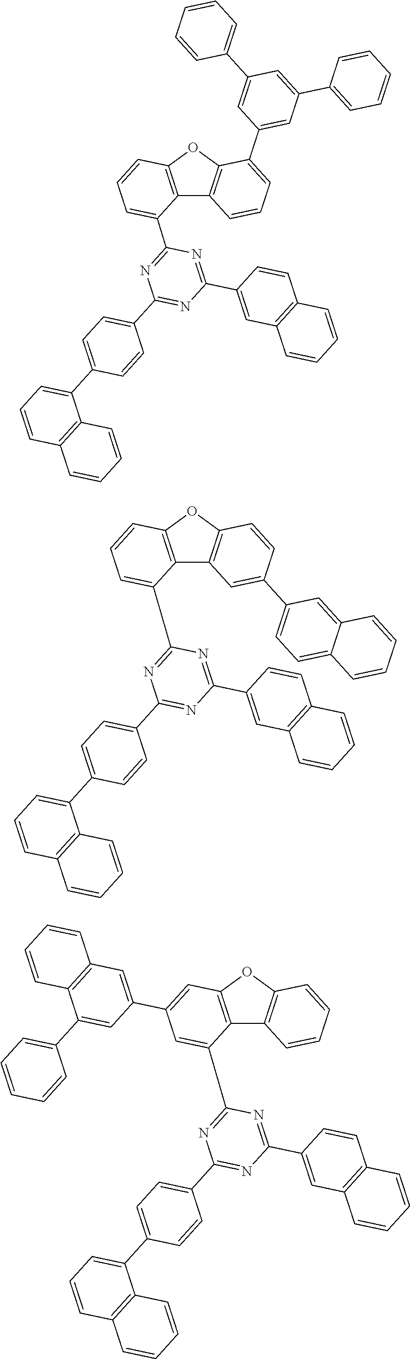

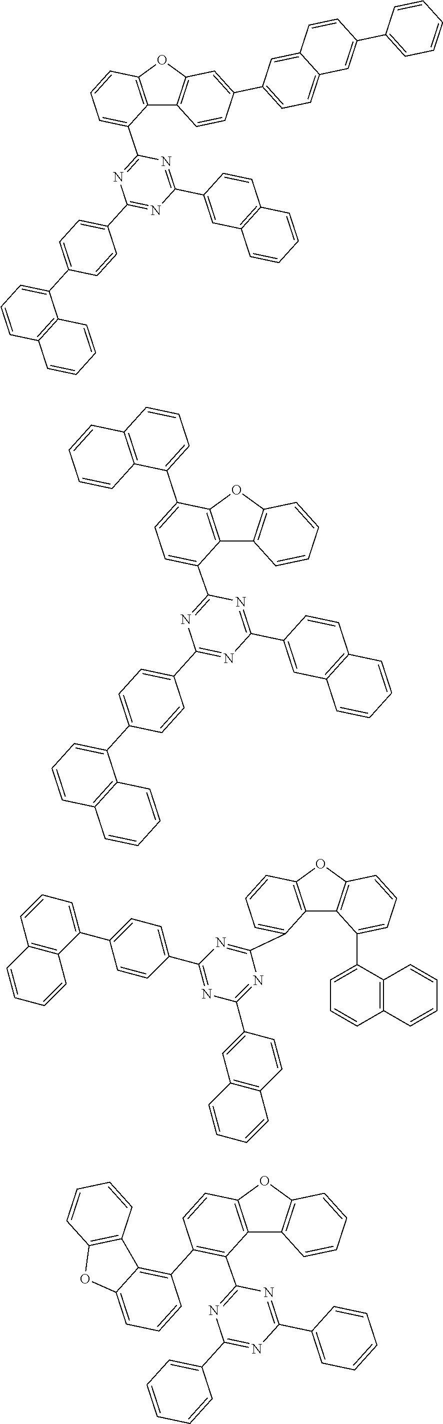

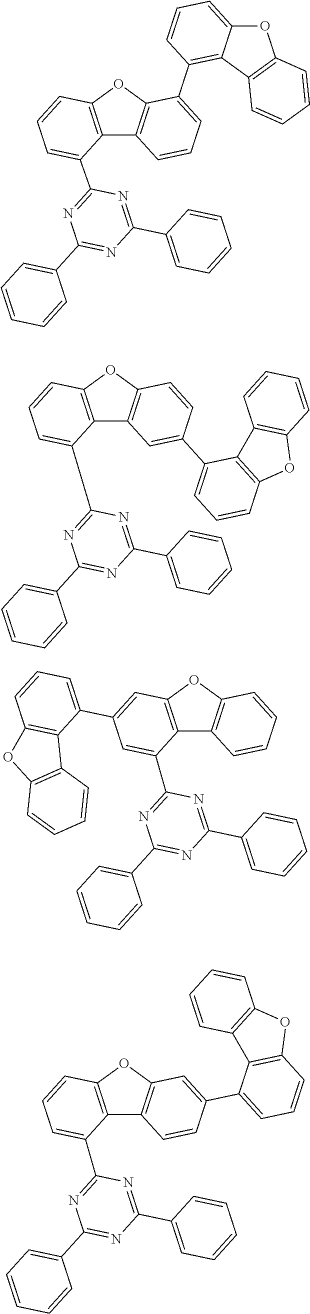

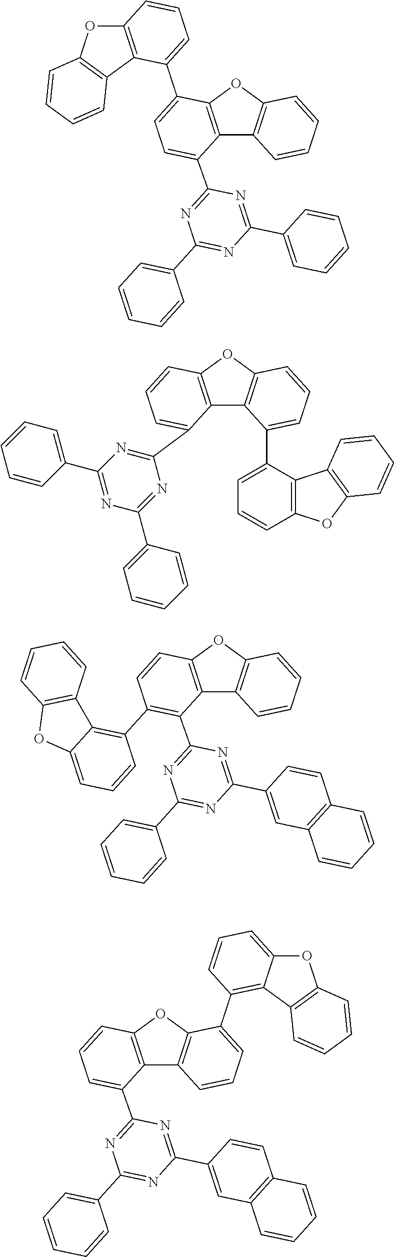

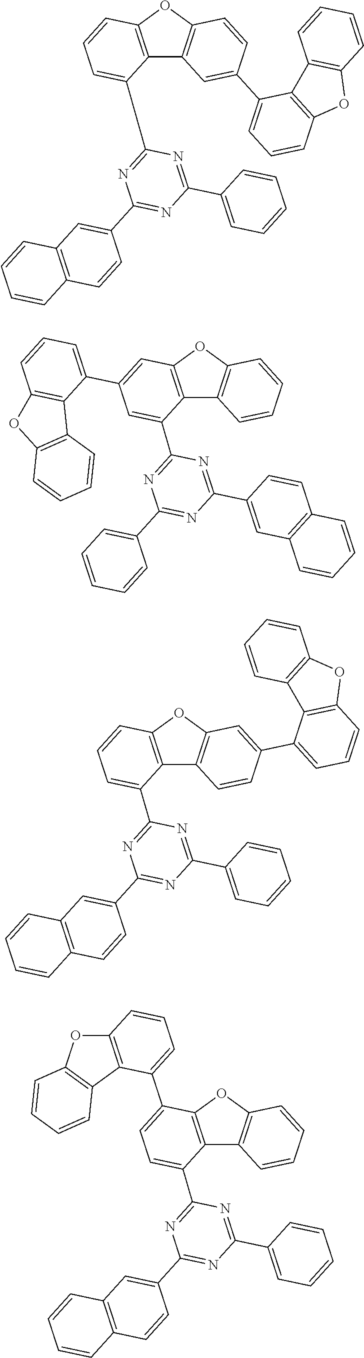

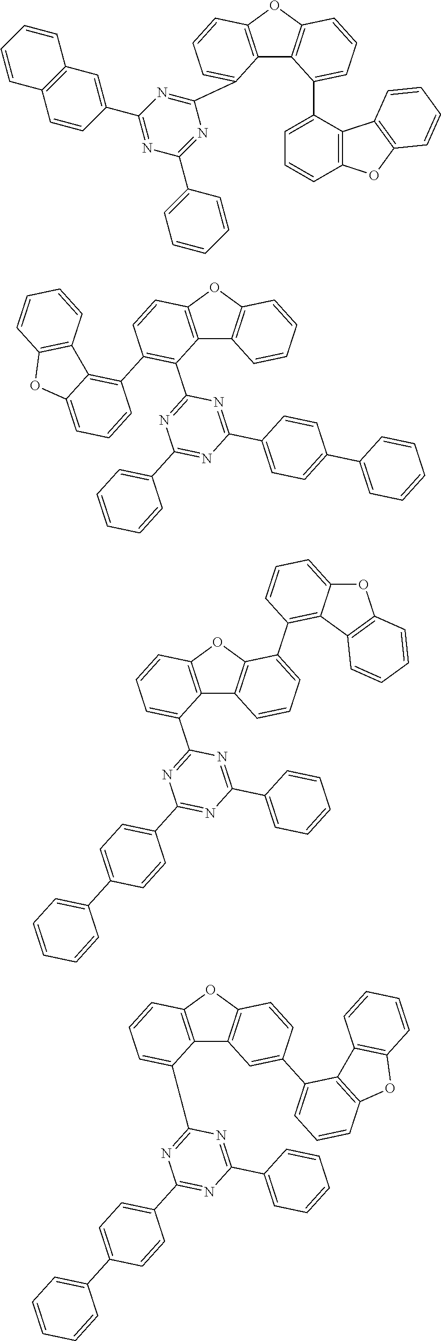

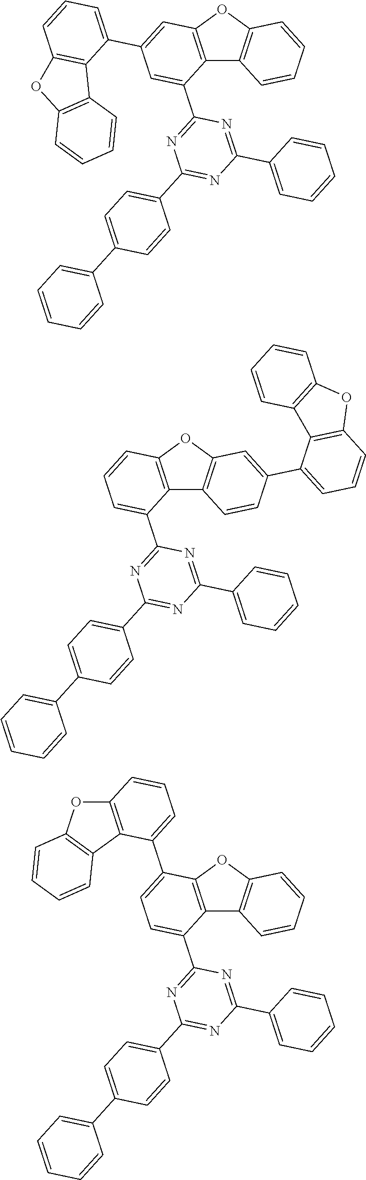

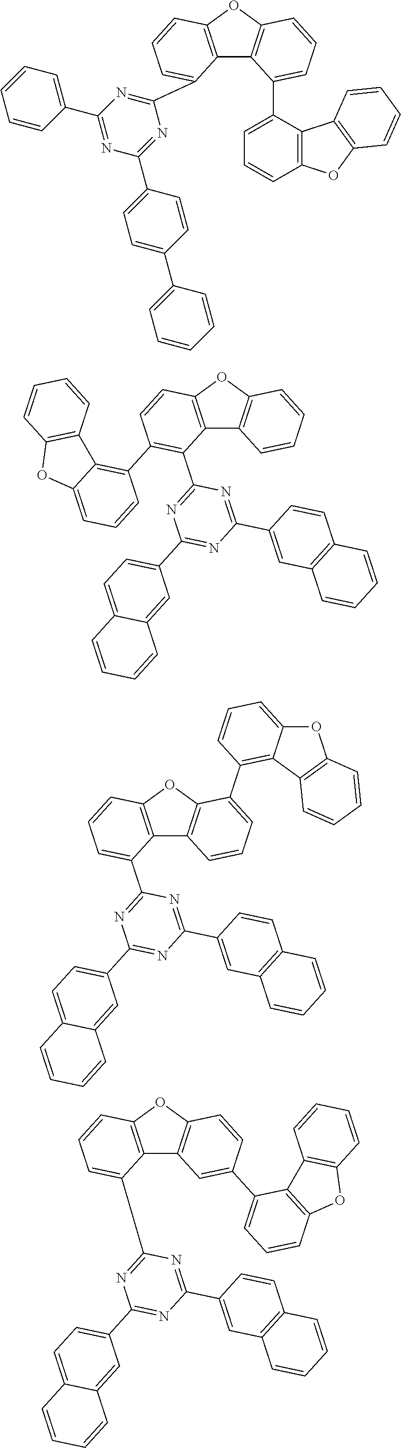

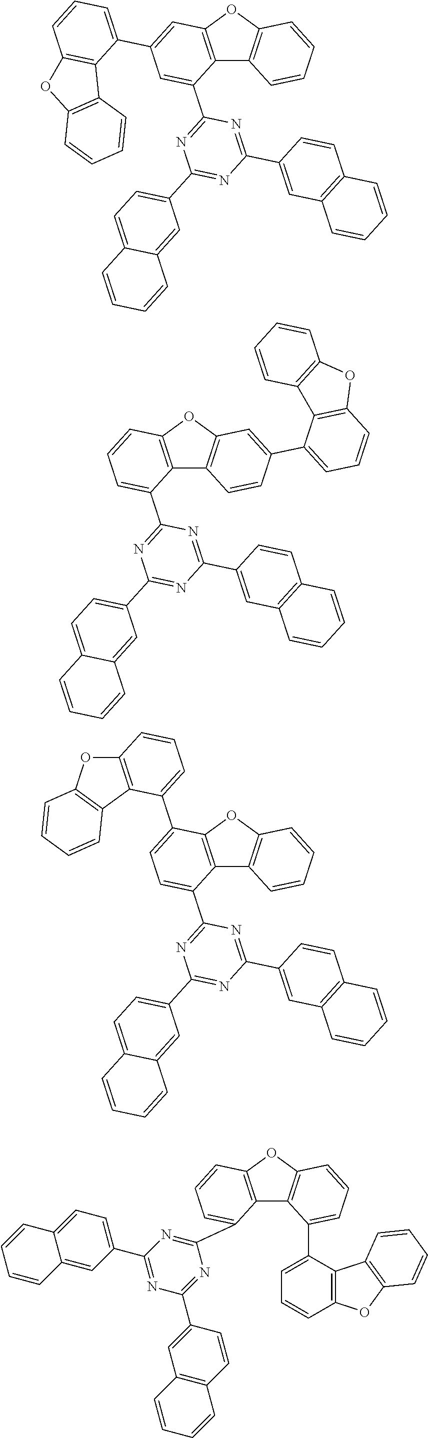

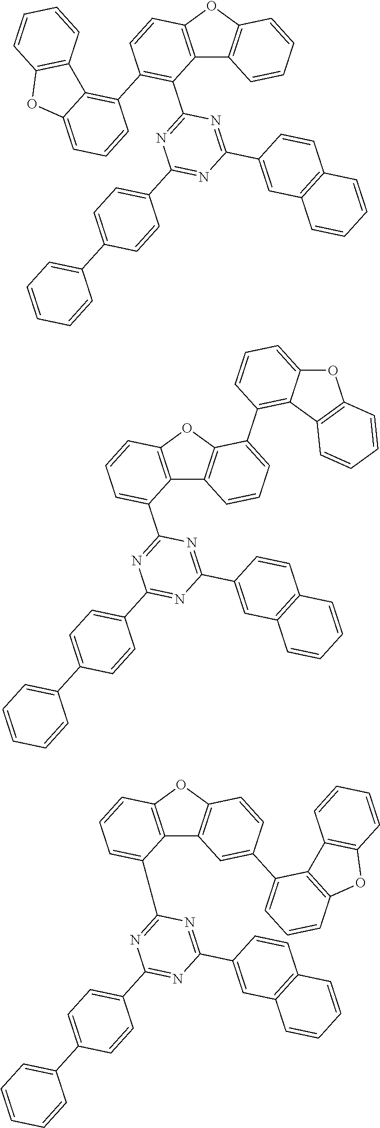

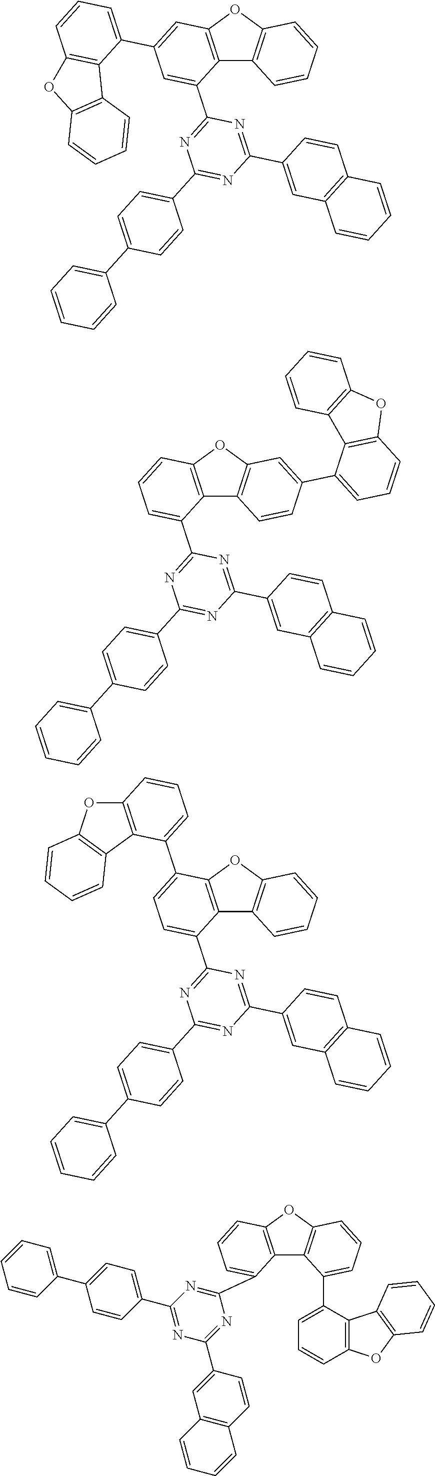

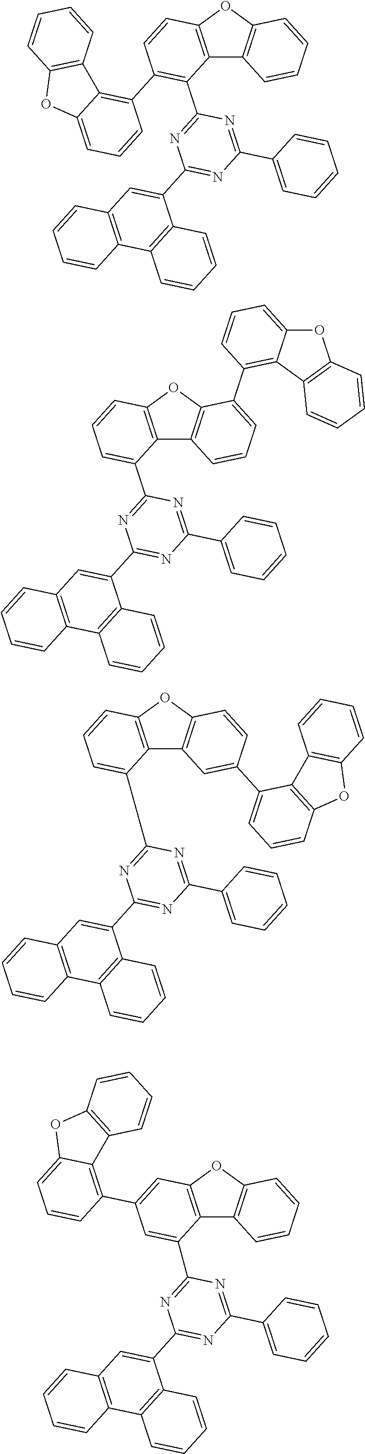

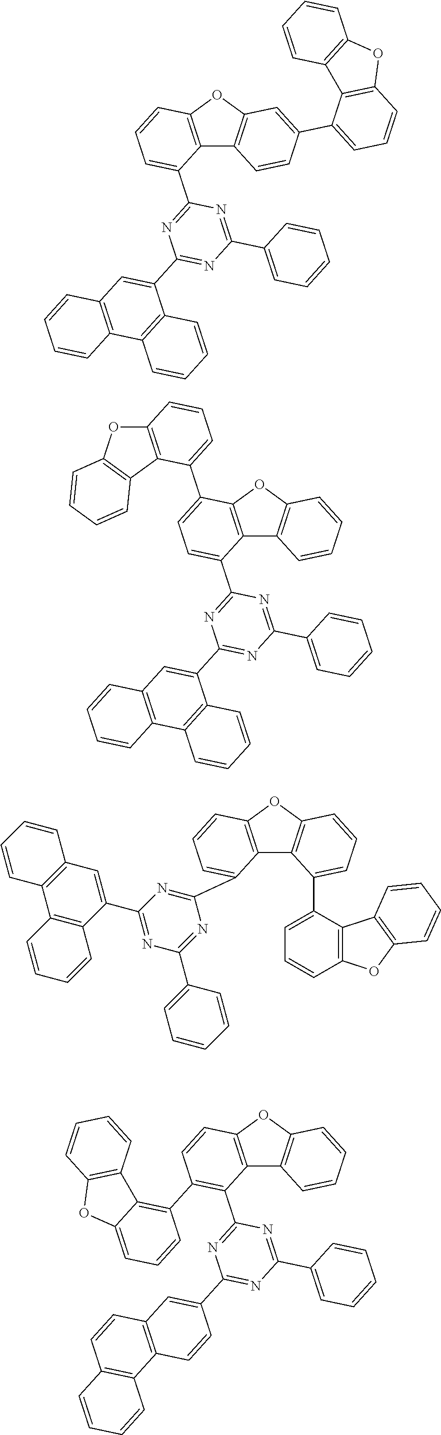

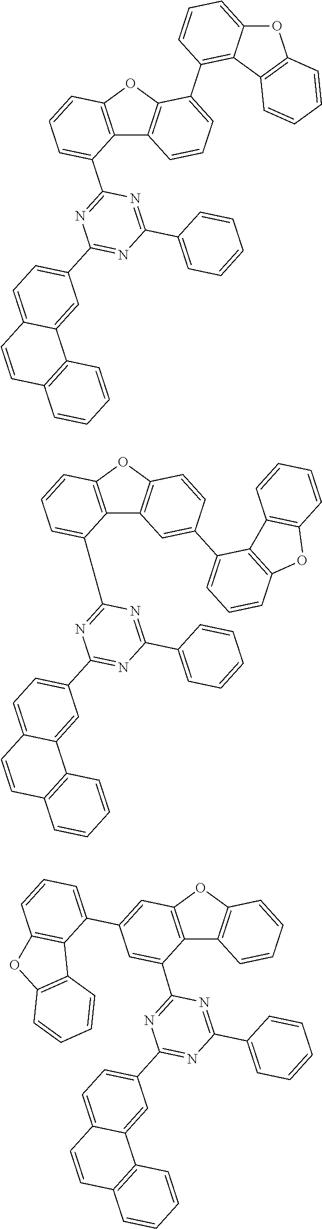

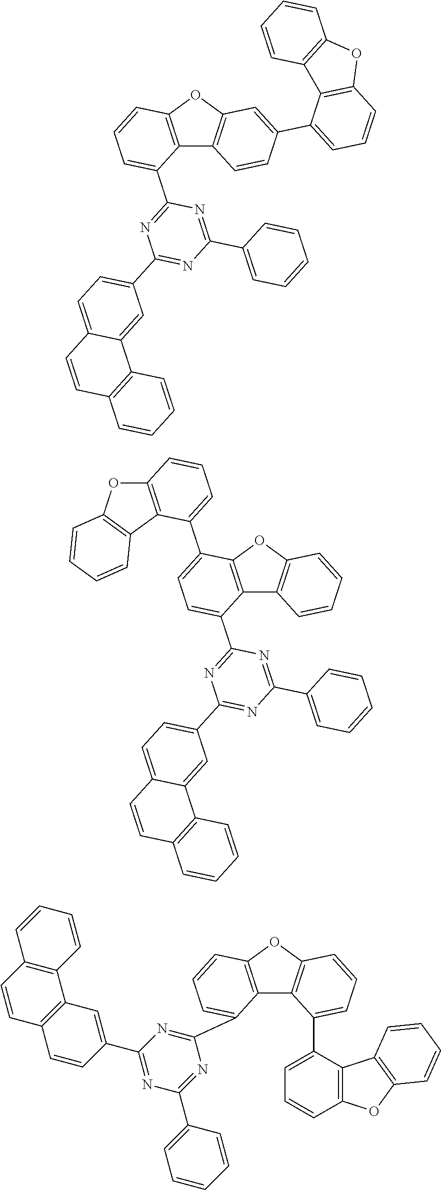

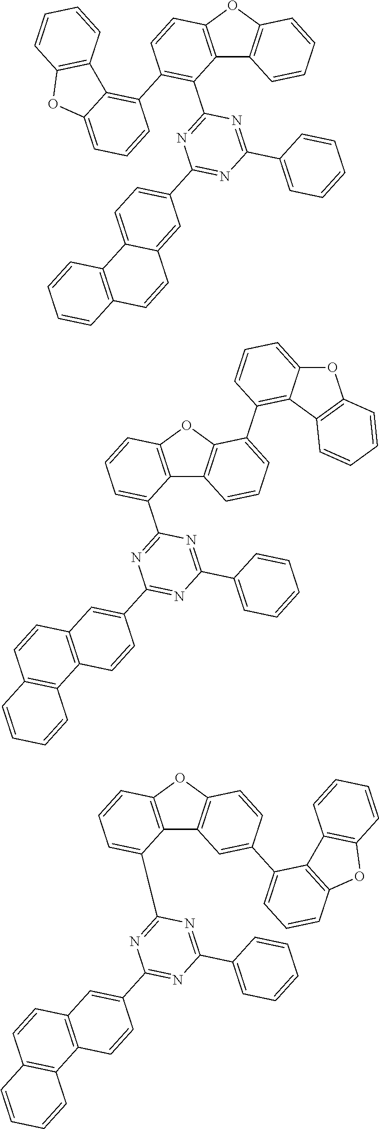

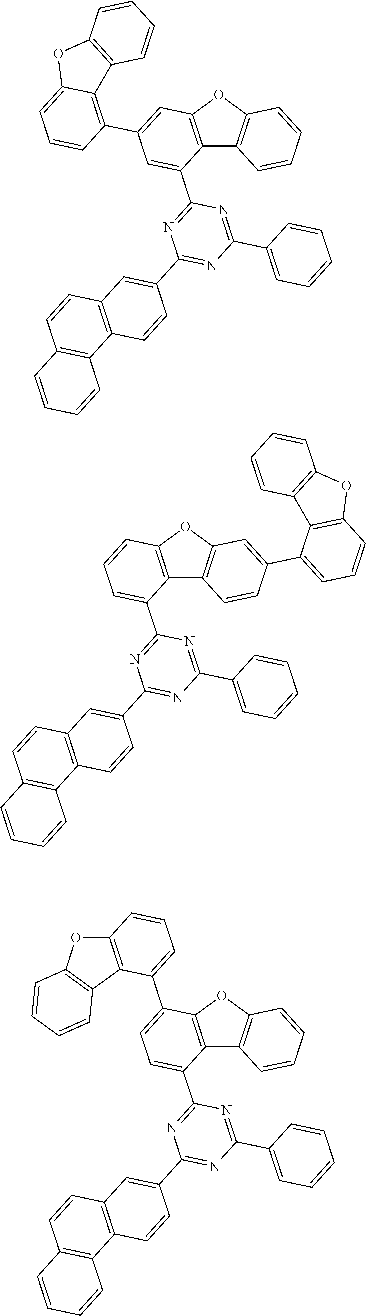

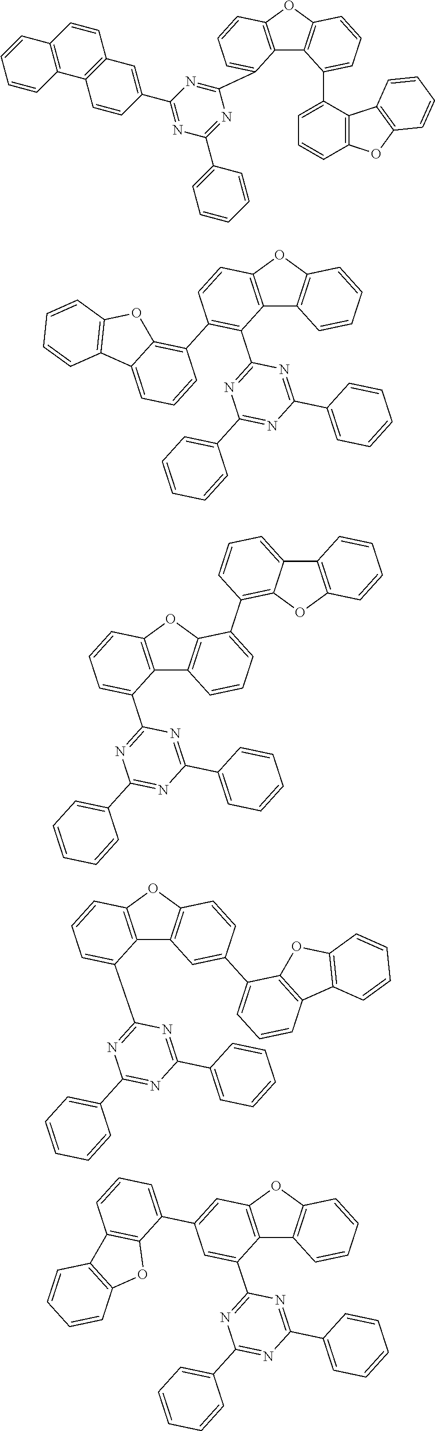

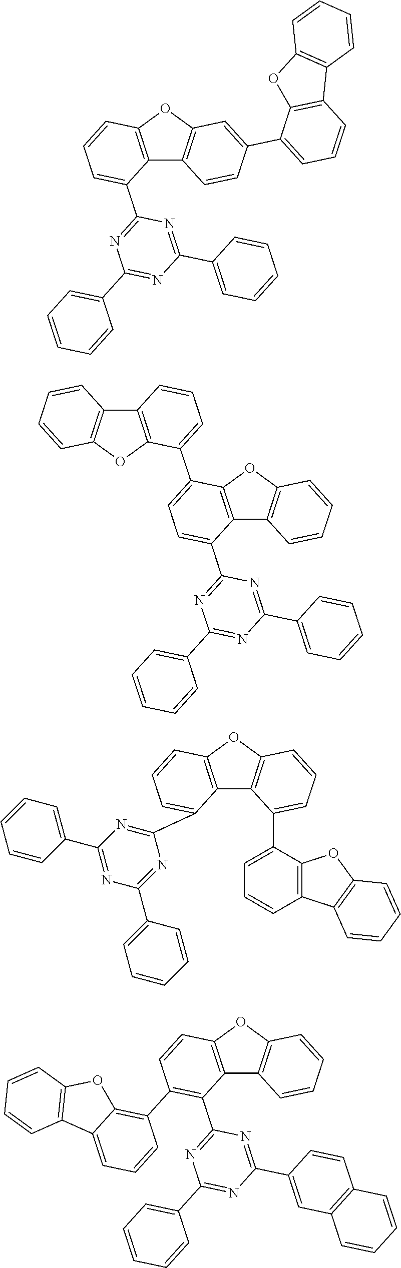

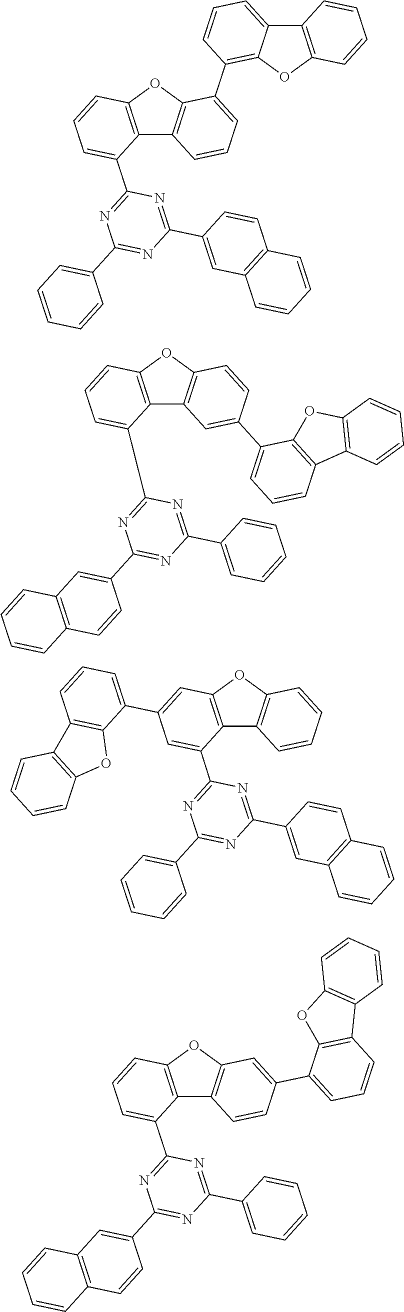

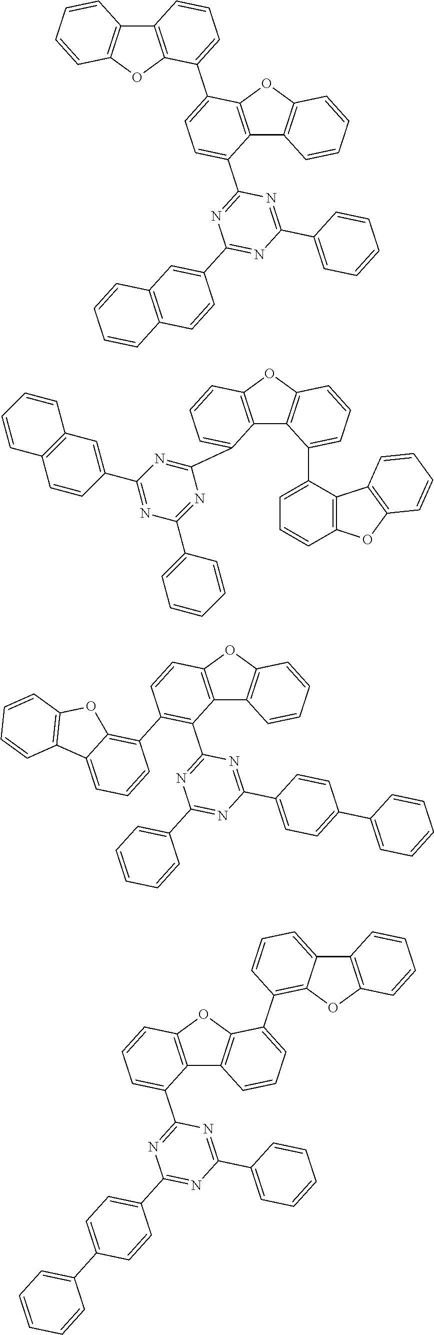

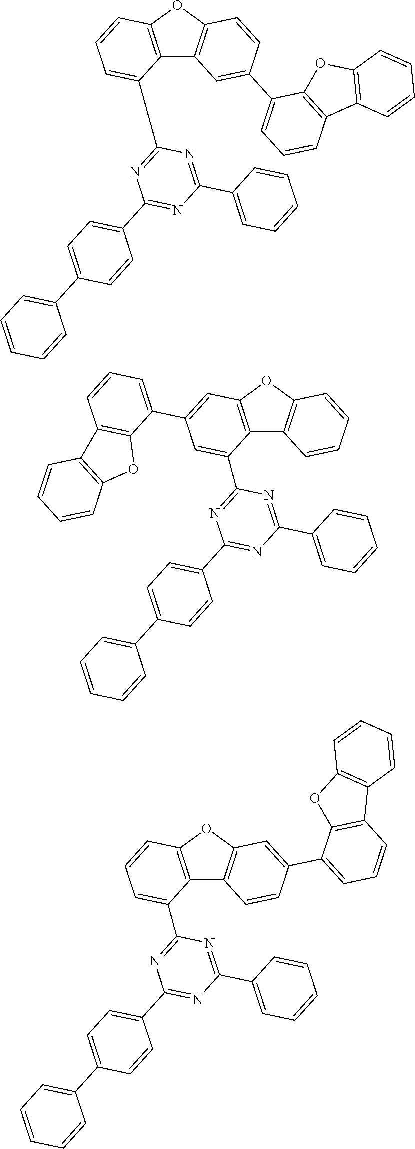

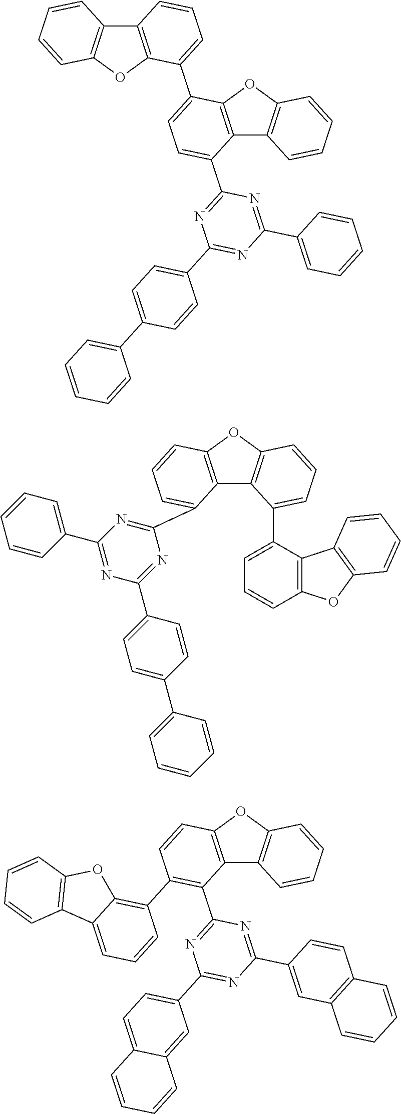

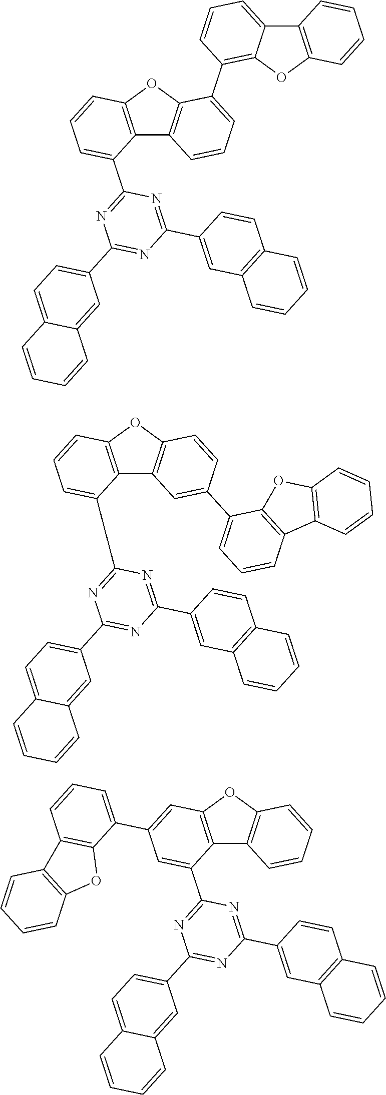

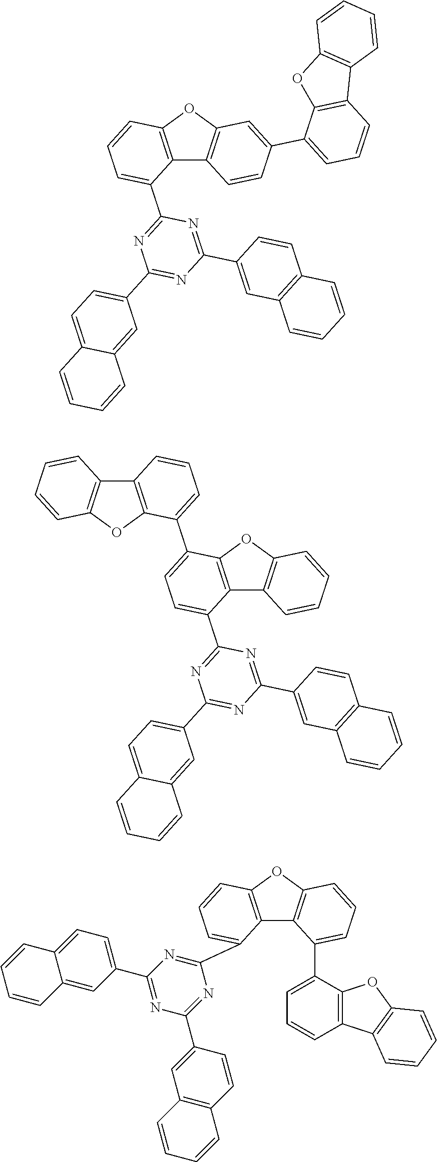

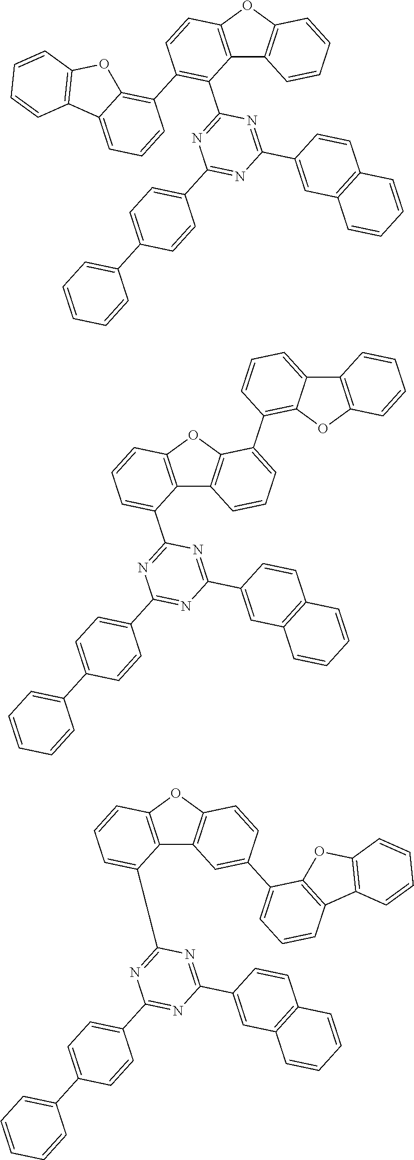

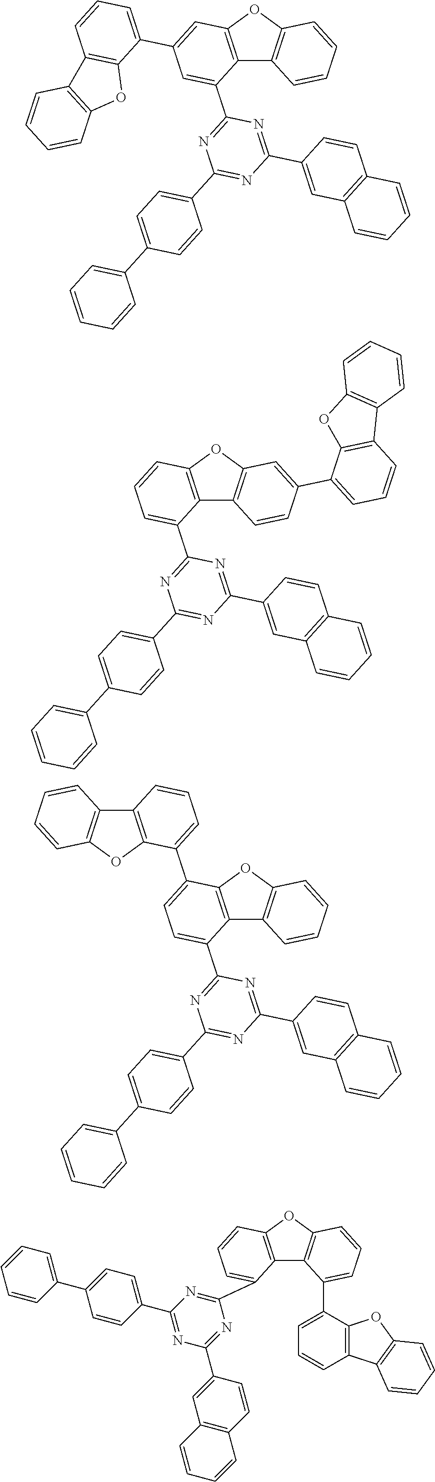

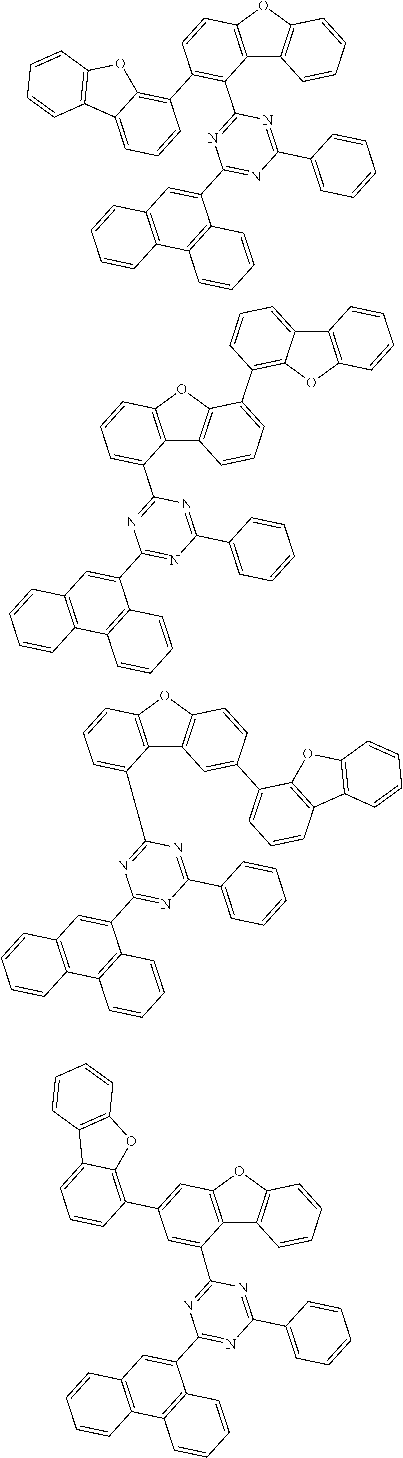

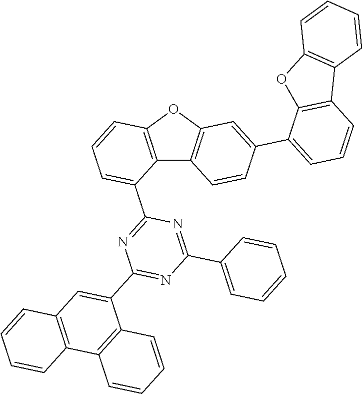 [SMILES: c1ccc(-c2nc(-c3cc4ccccc4c4ccccc34)nc(-c3cccc4oc5cc(-c6cccc7c6oc6ccccc67)ccc5c34)n2)cc1]

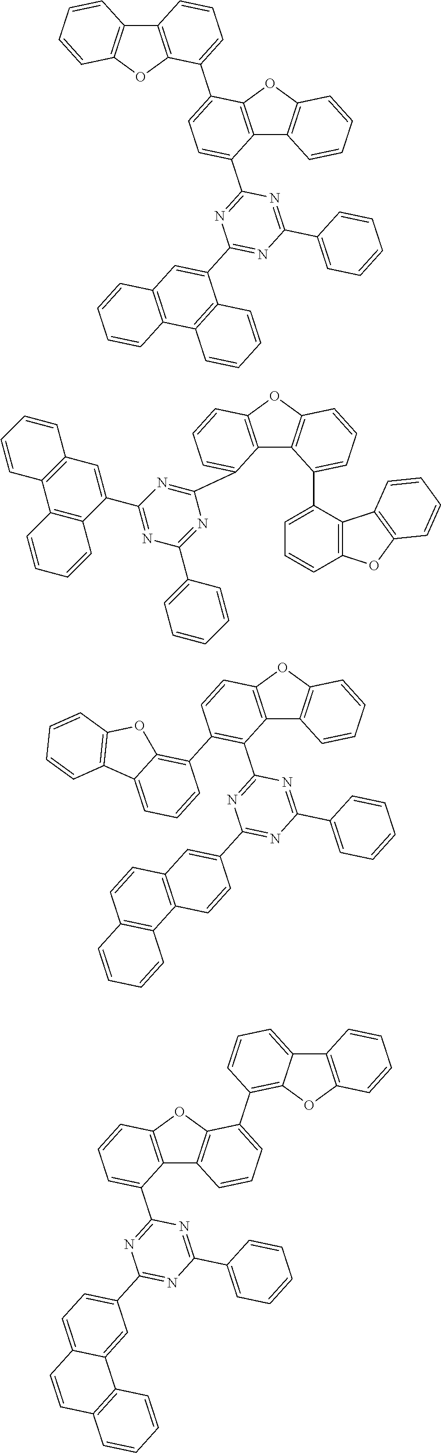

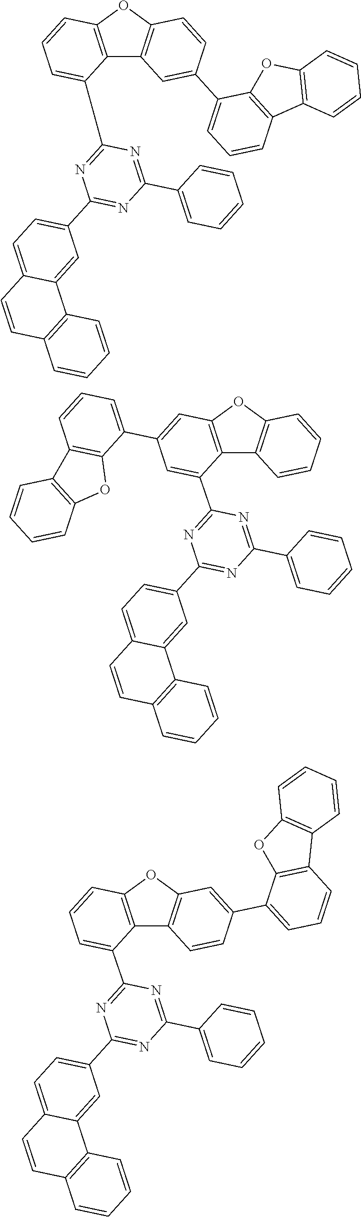

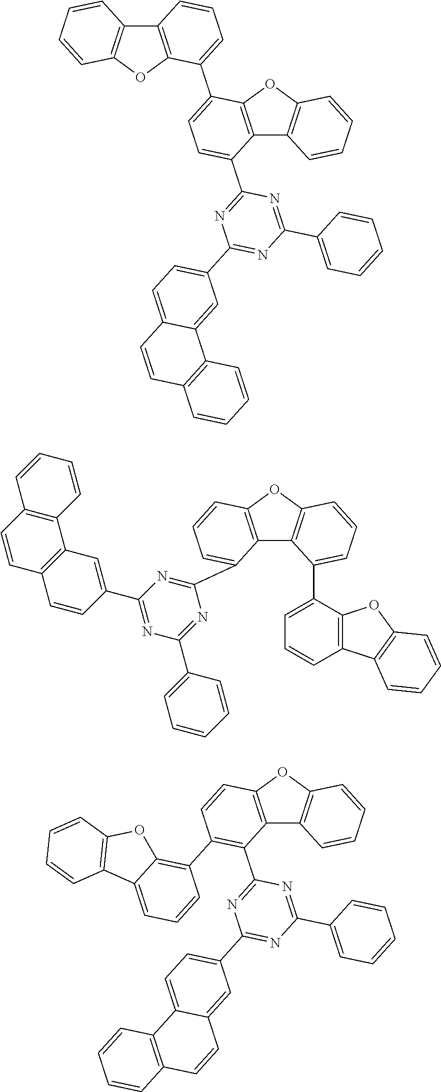

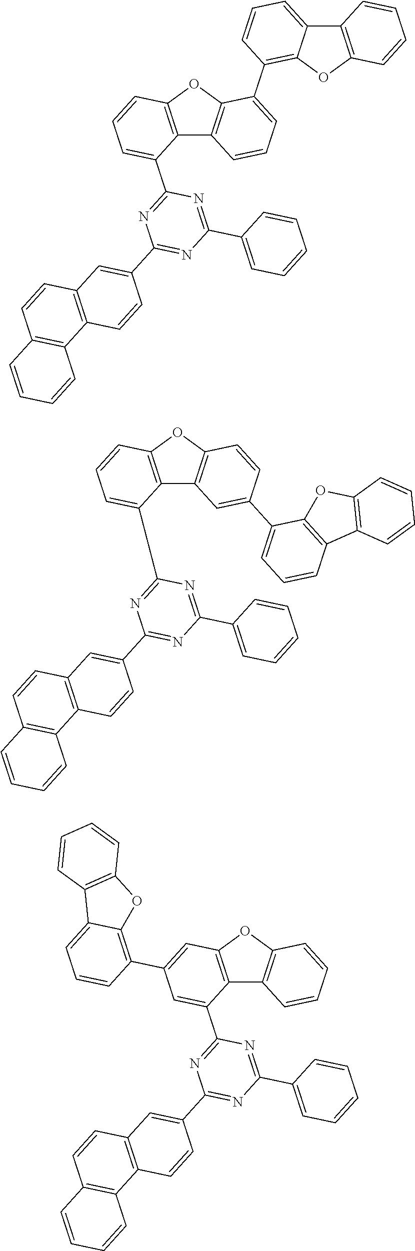

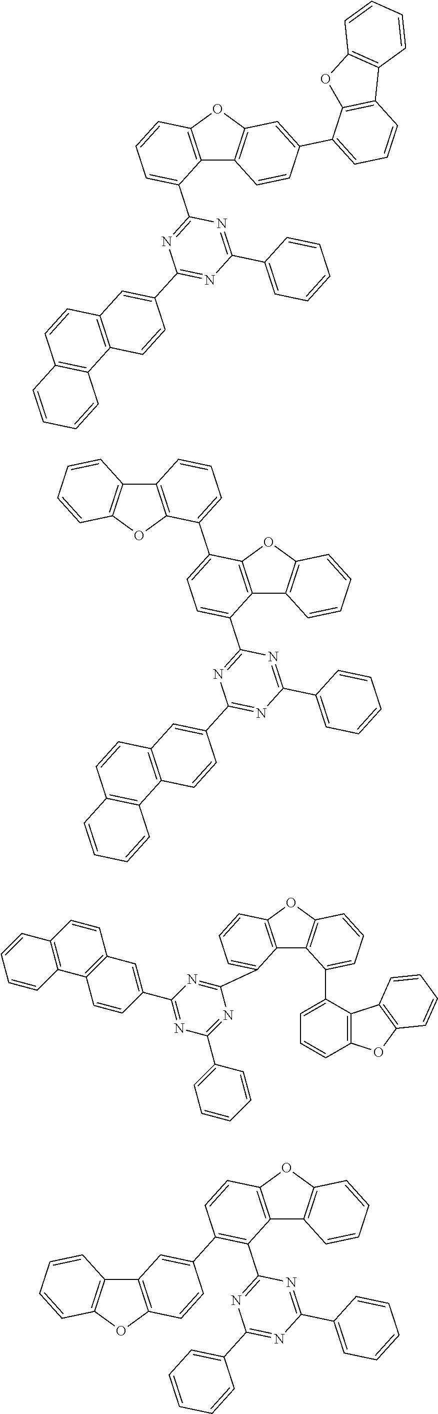

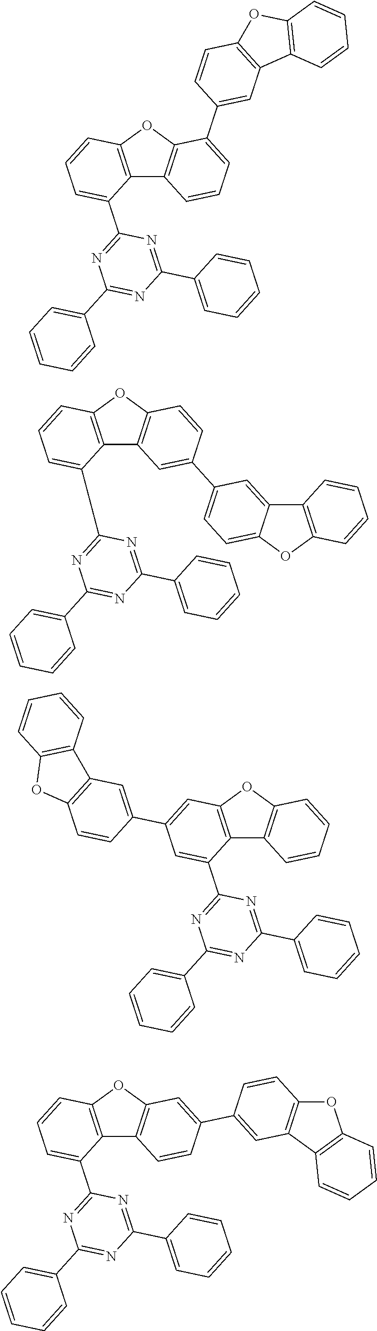

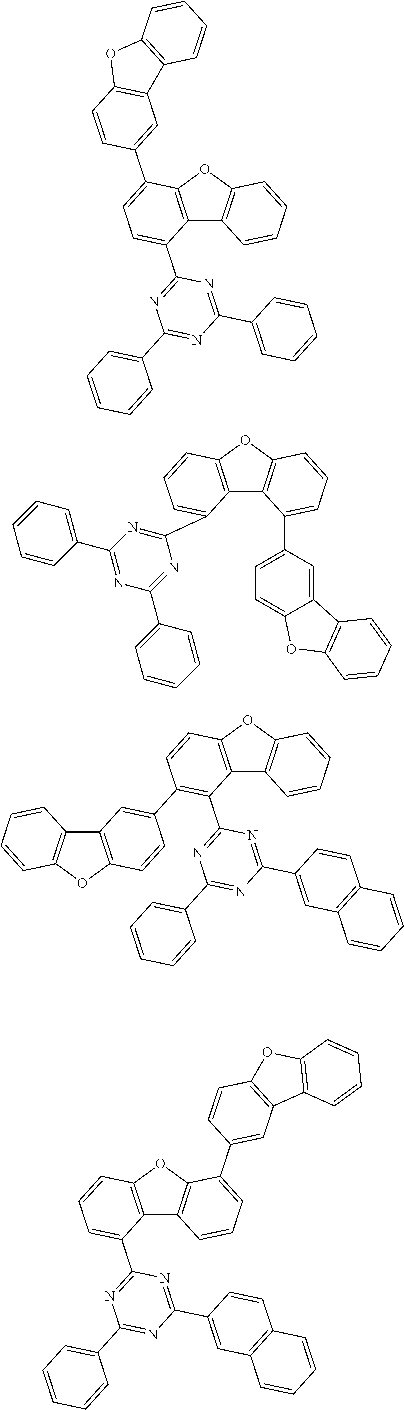

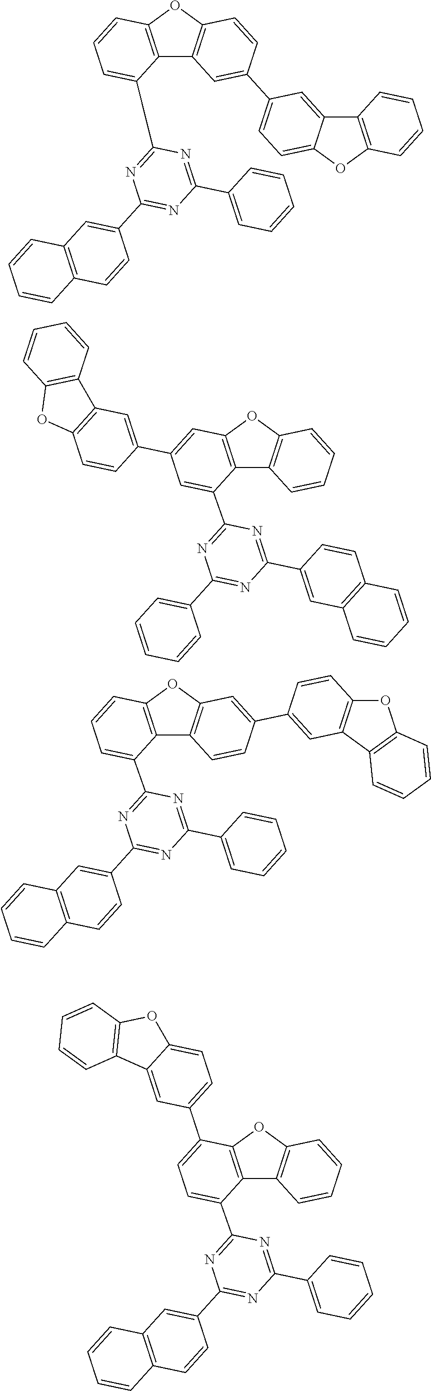

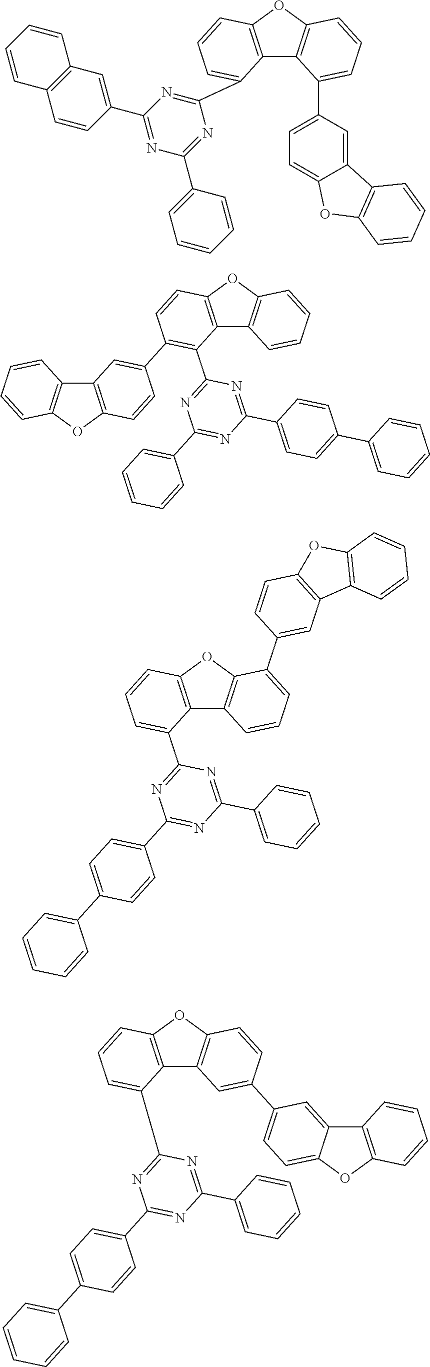

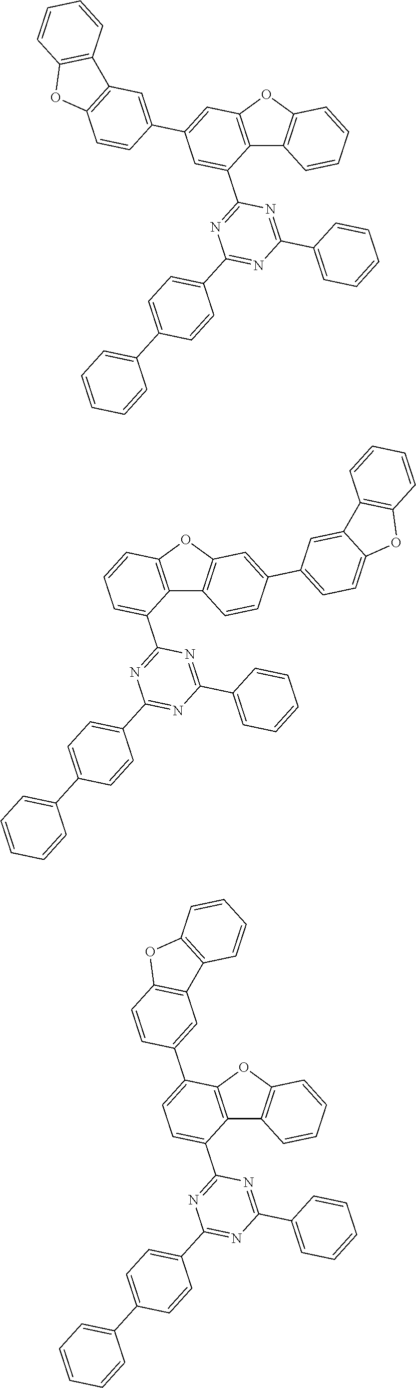

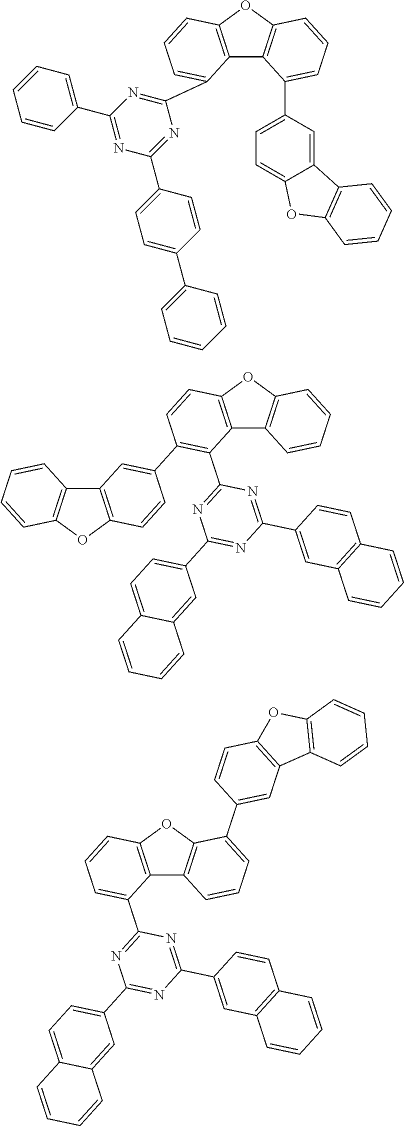

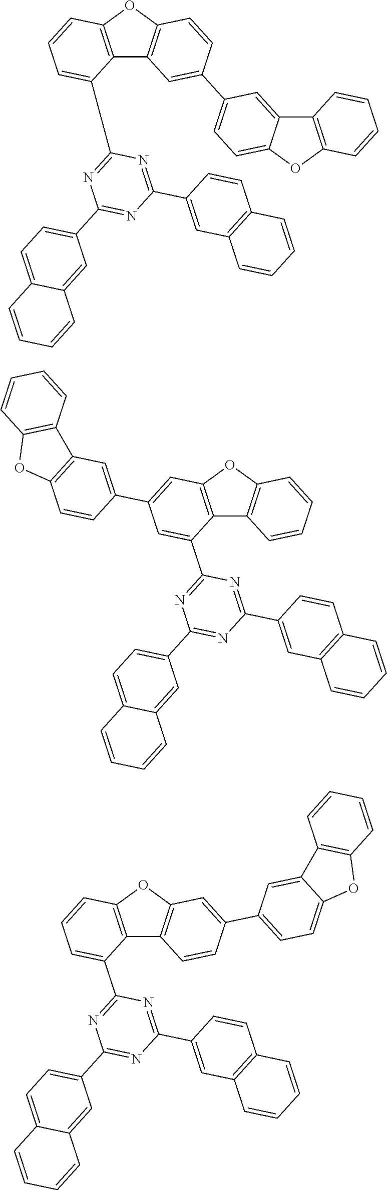

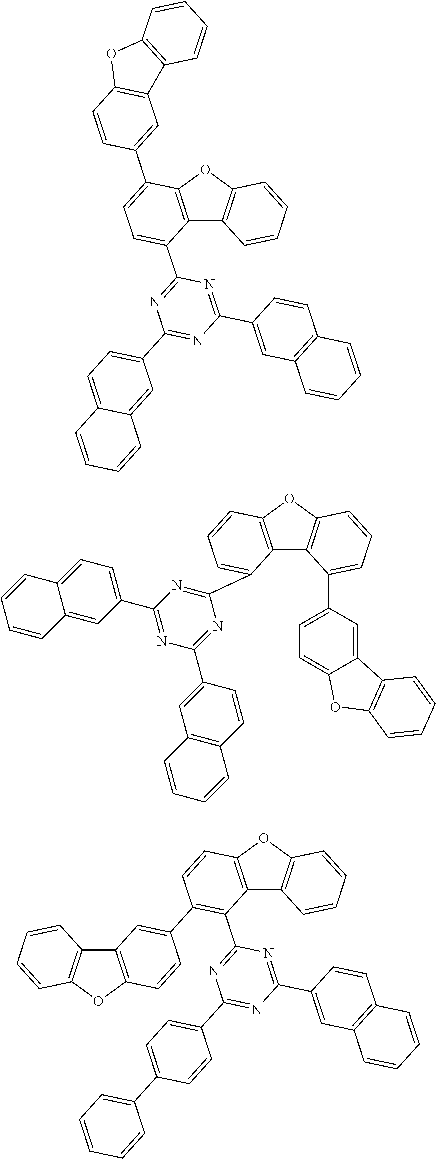

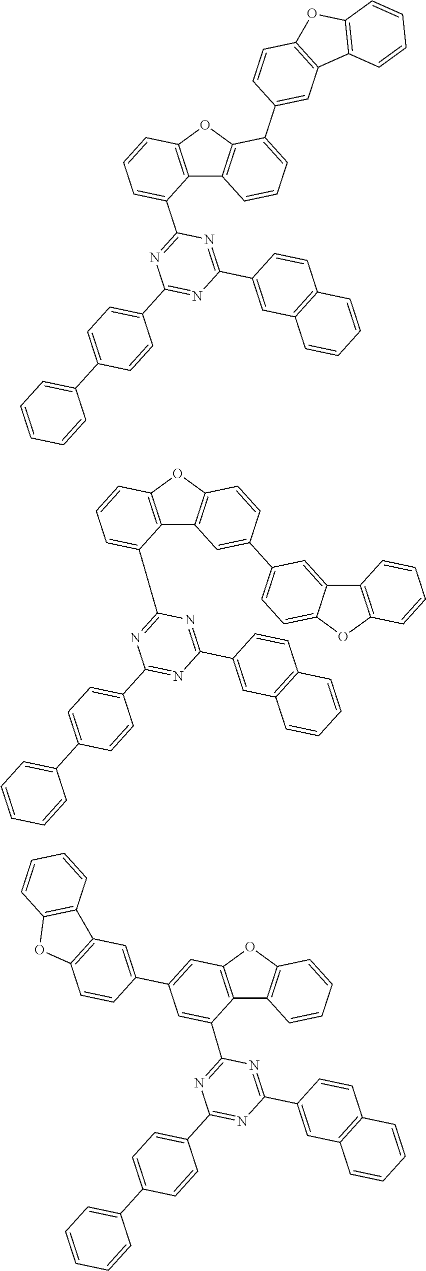

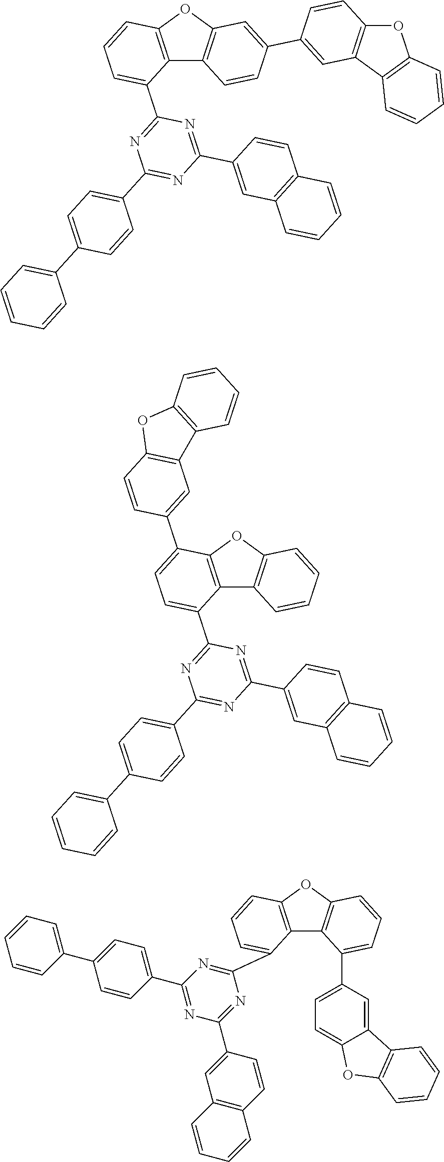

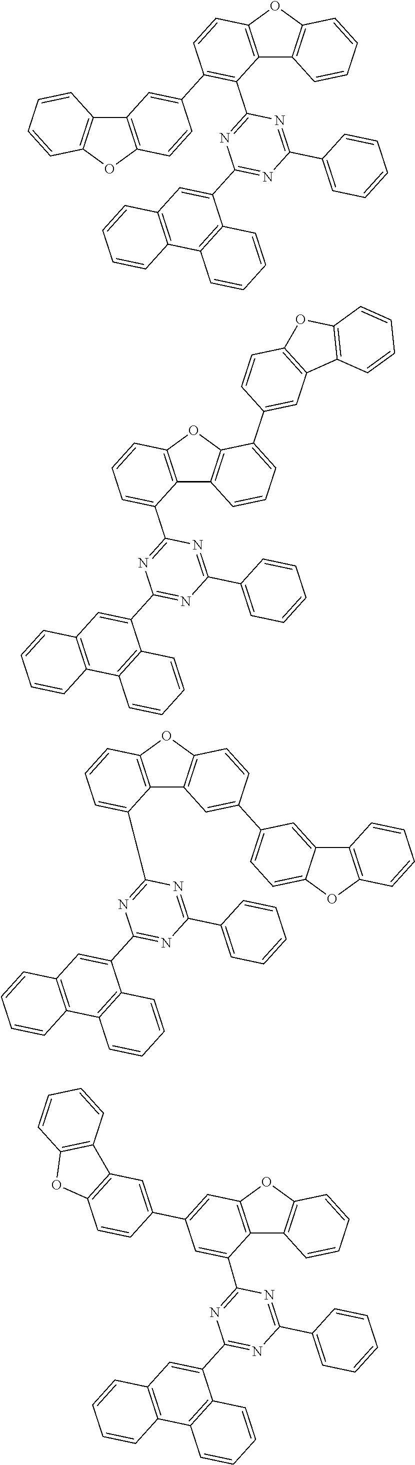

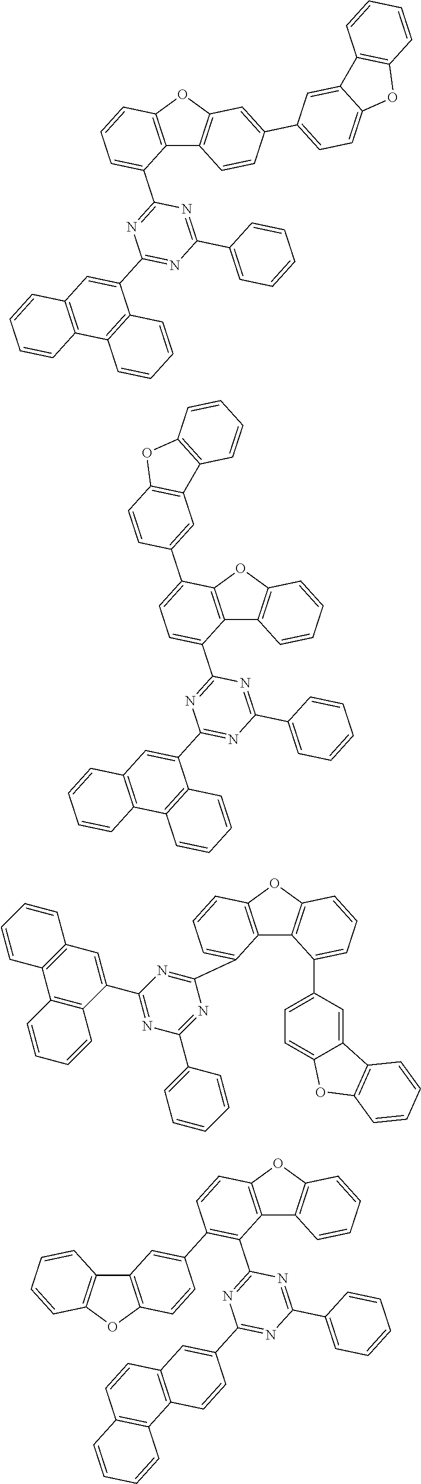

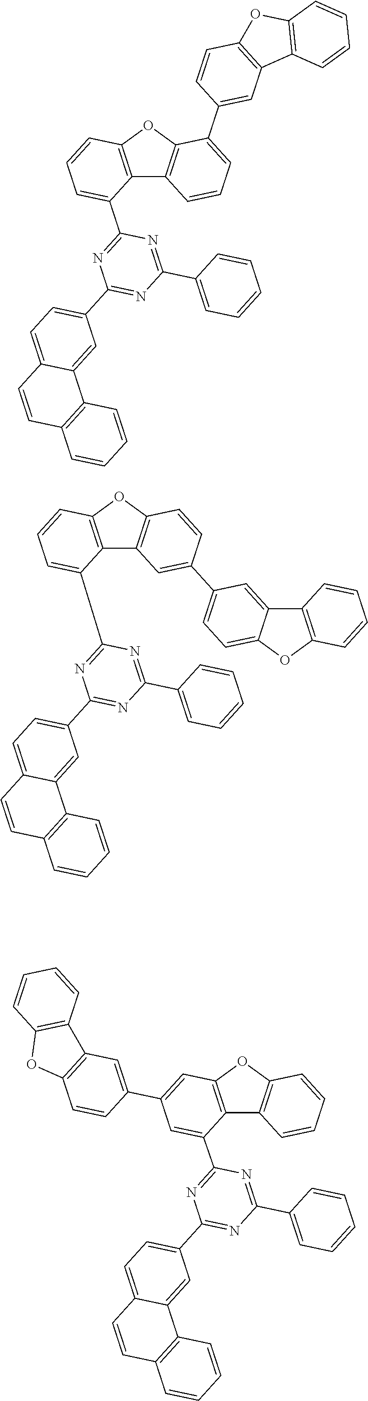

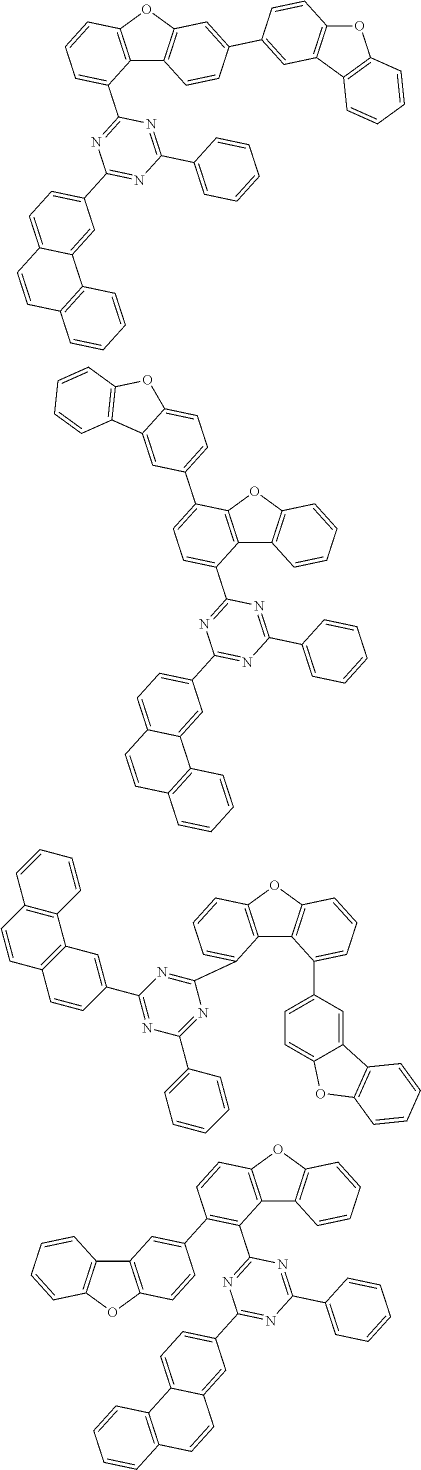

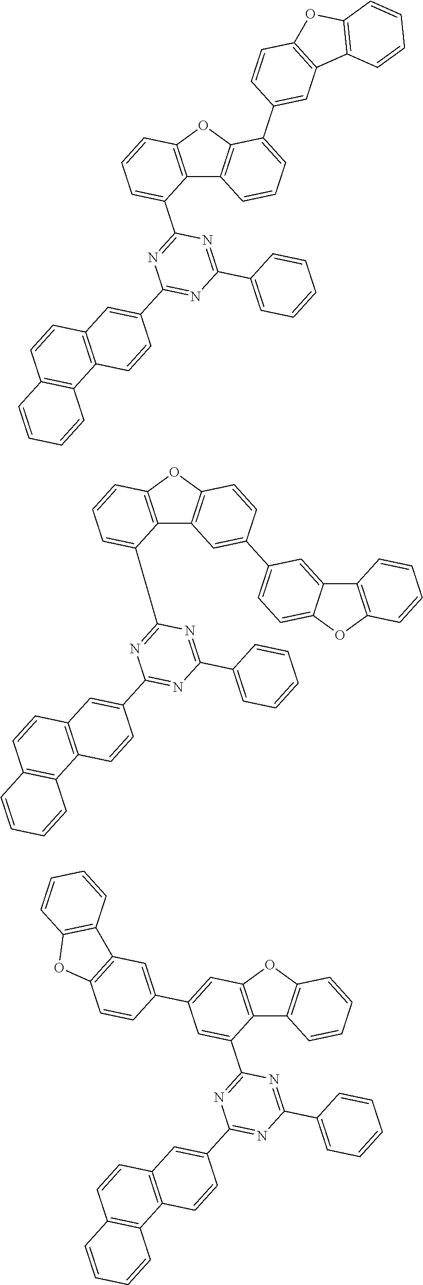

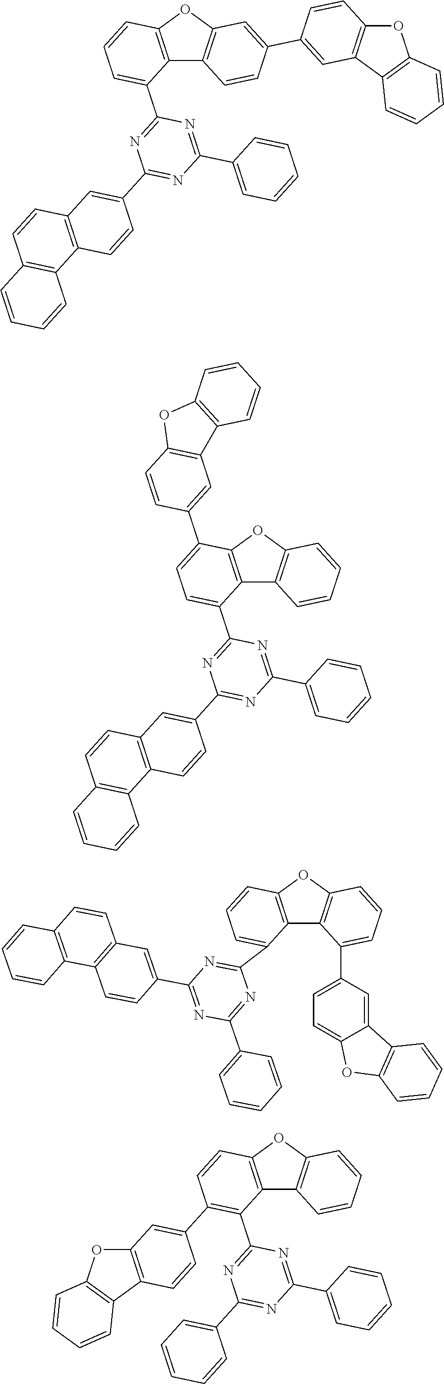

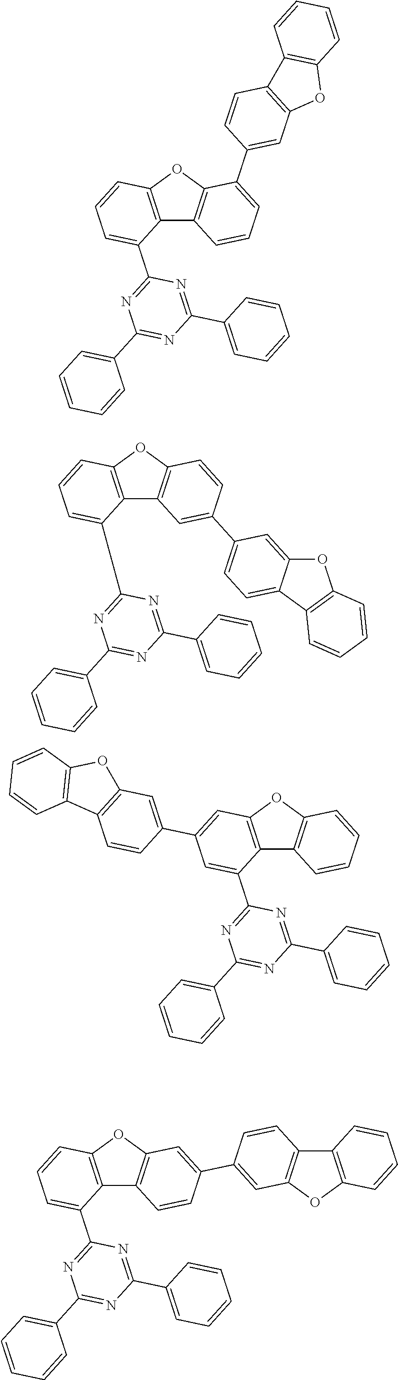

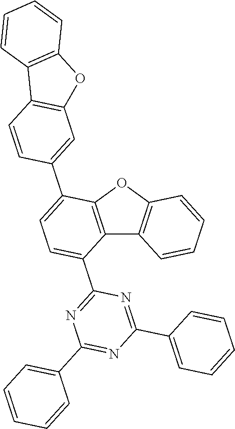

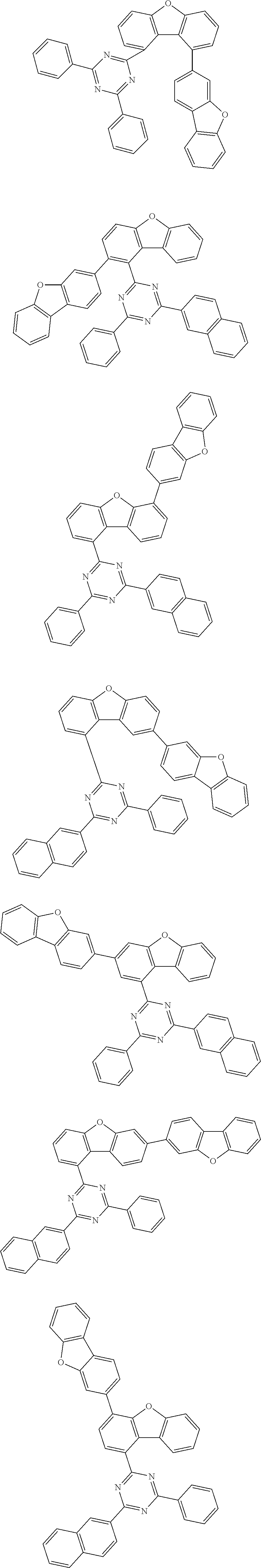

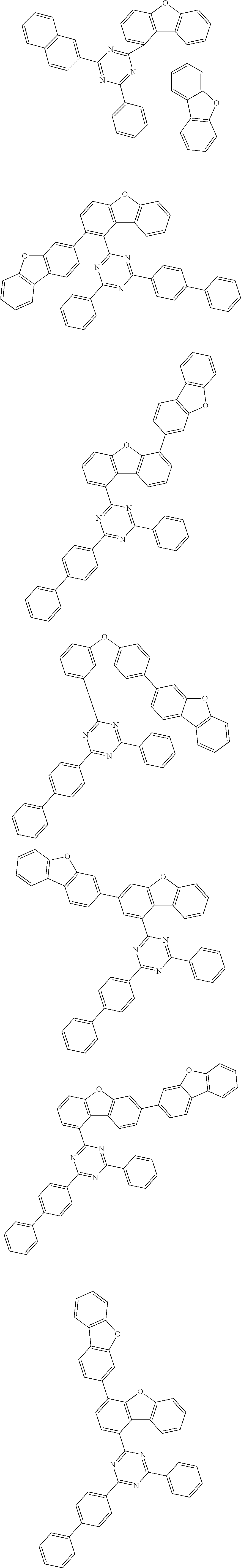

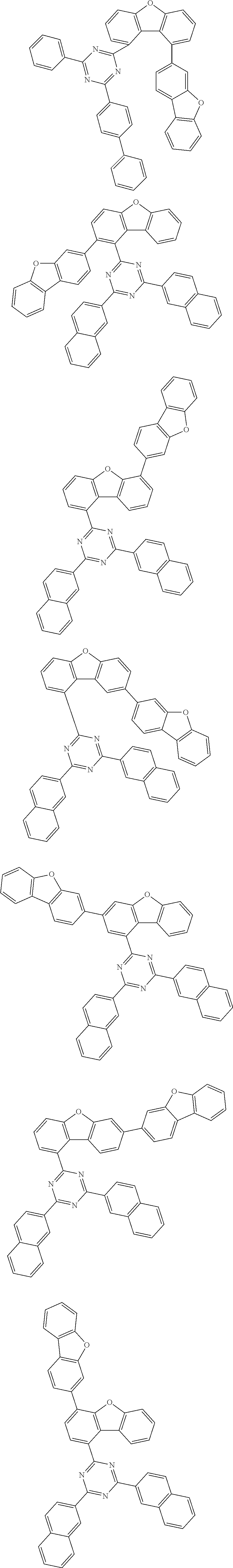

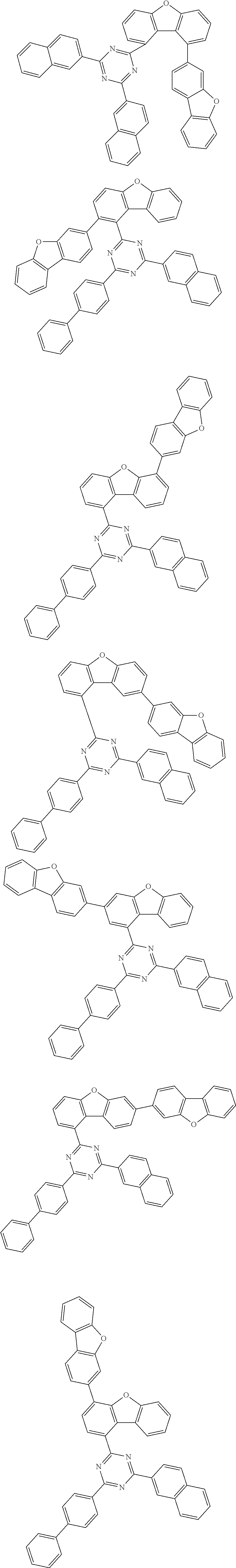

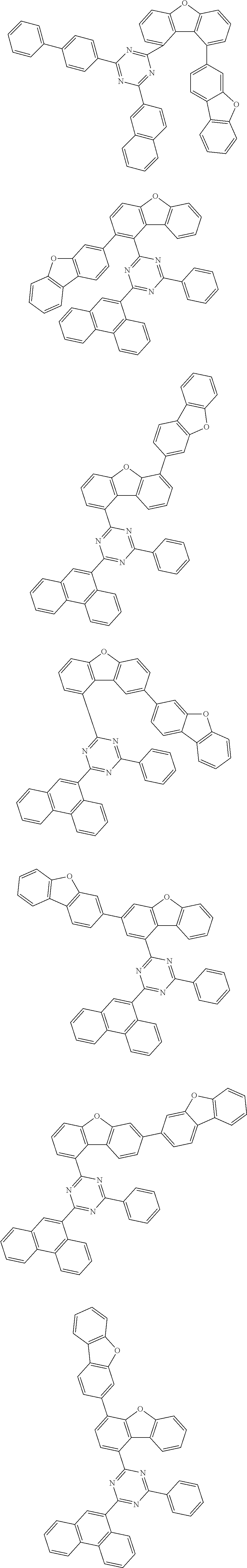

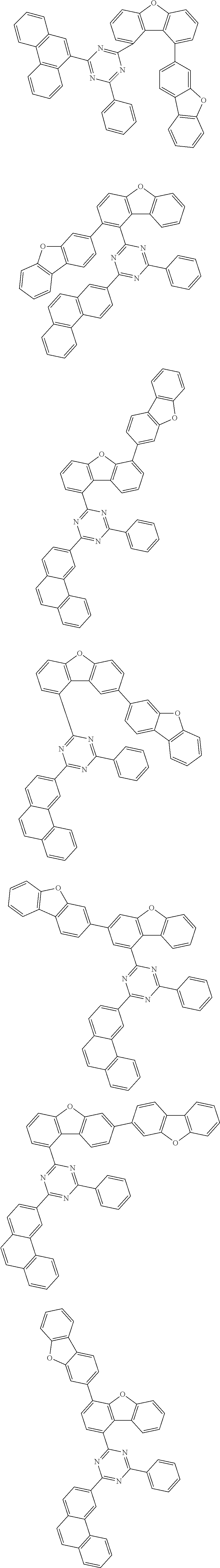

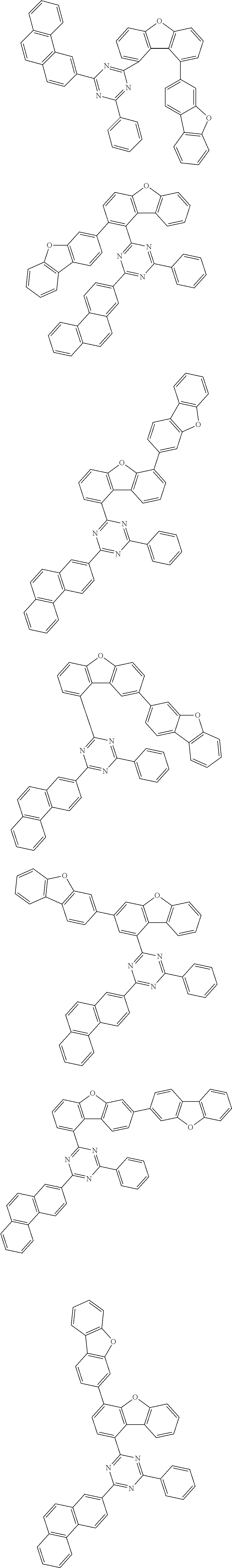

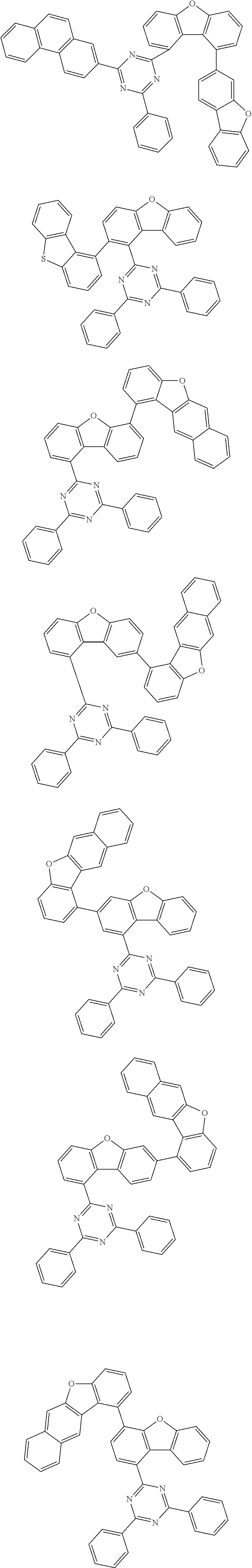

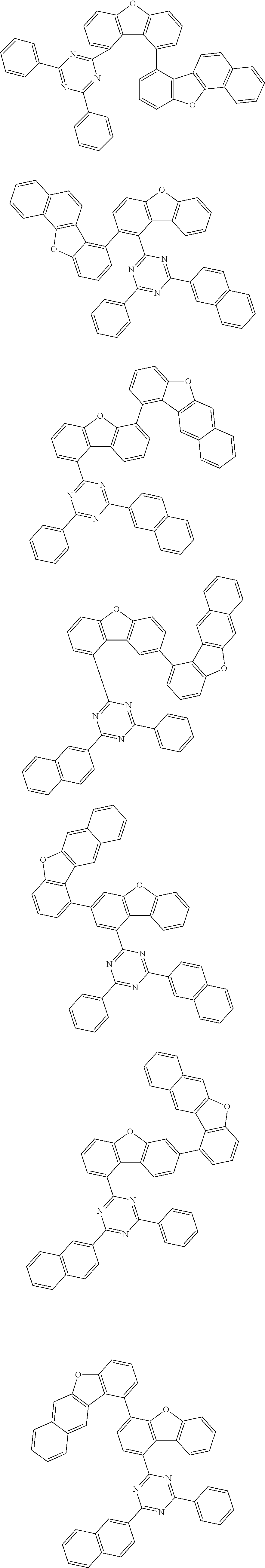

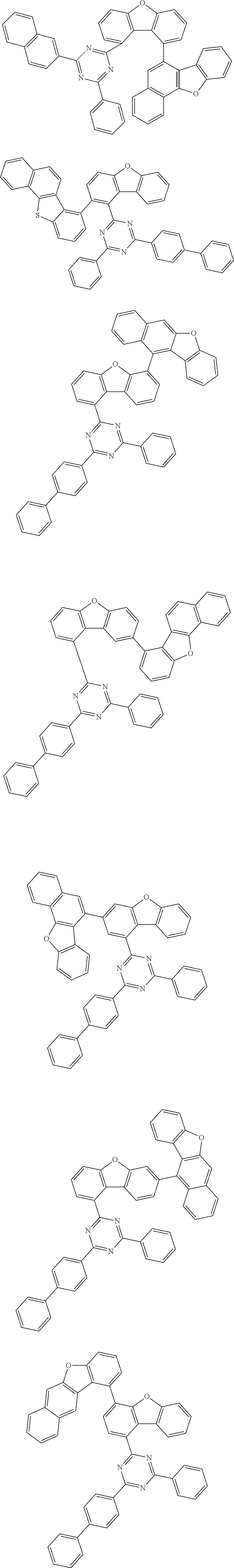

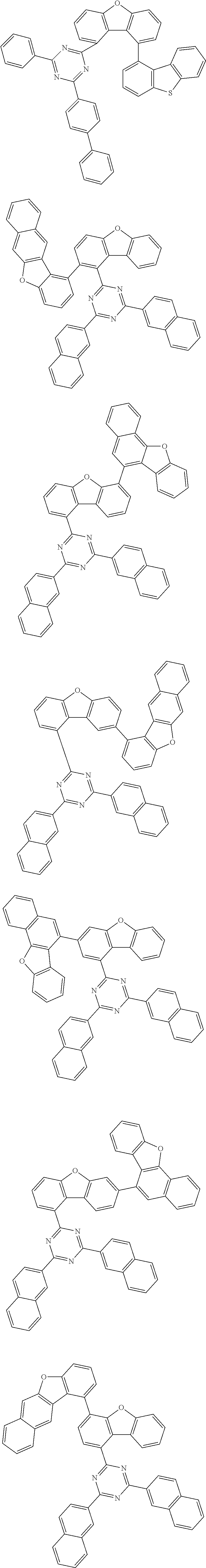

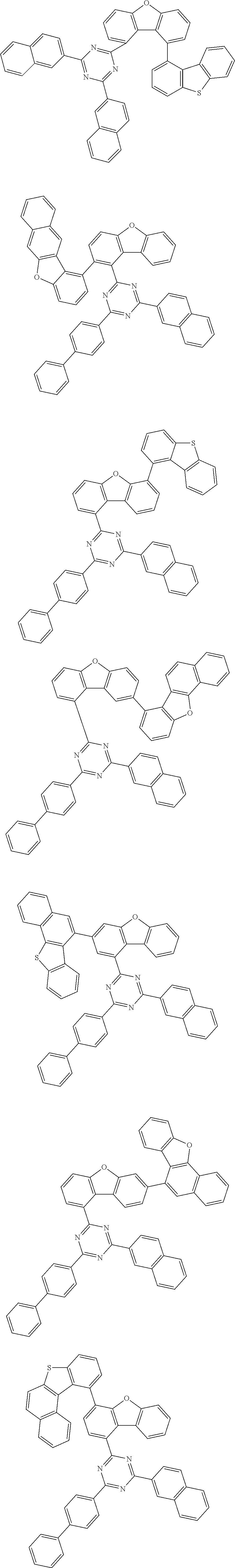

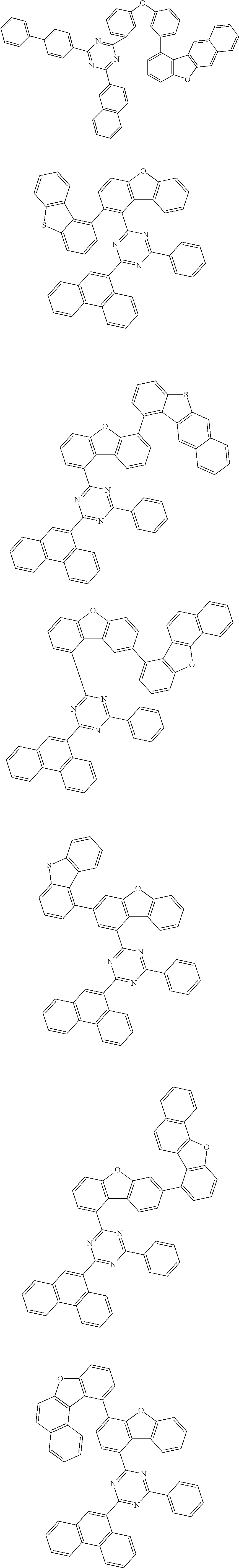

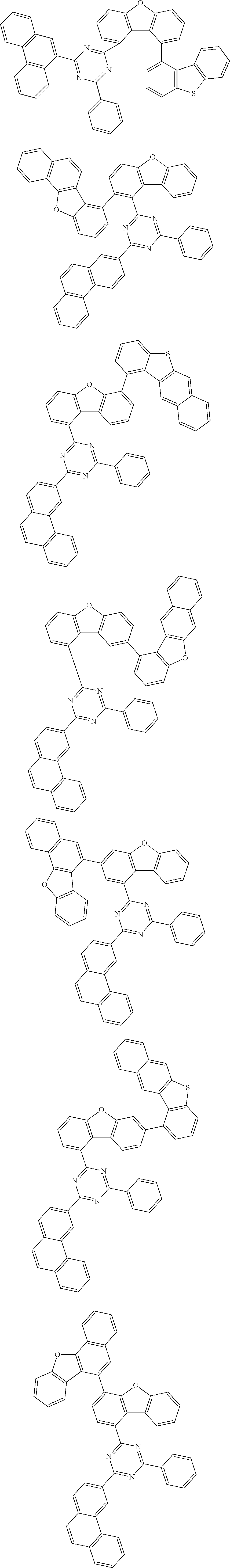

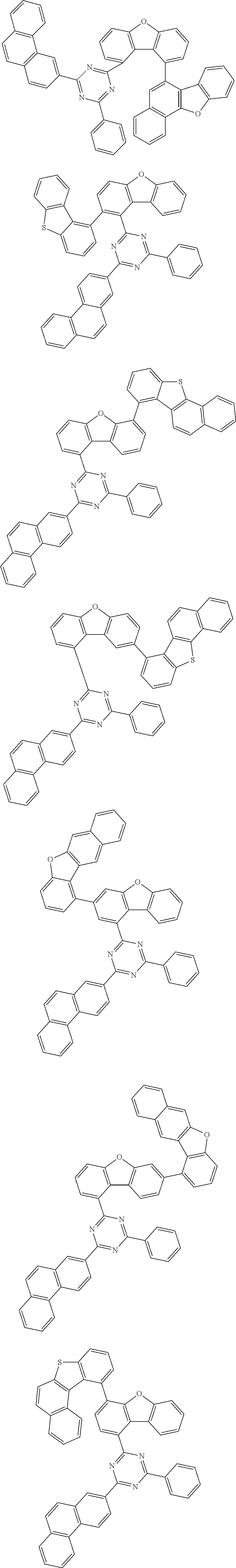

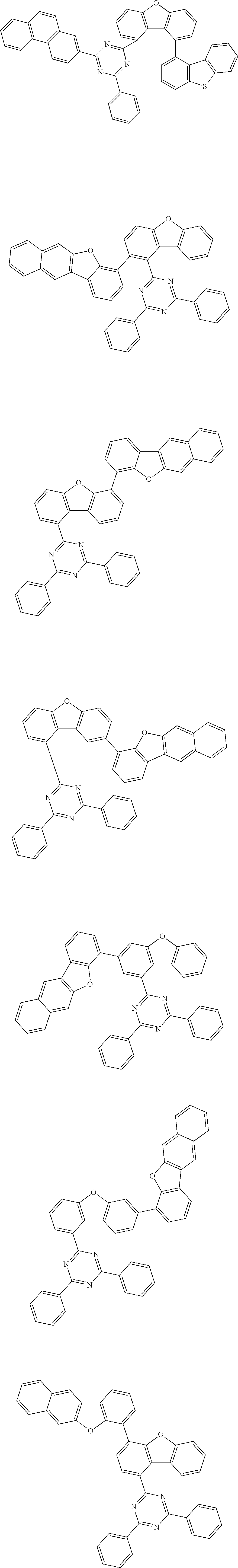

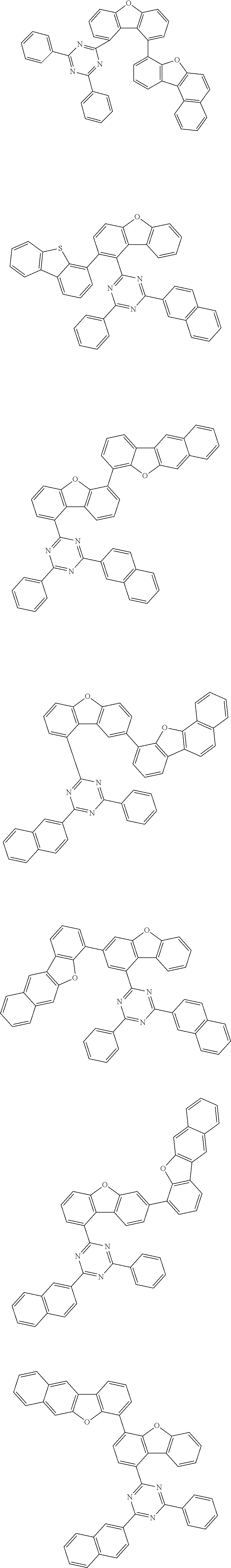

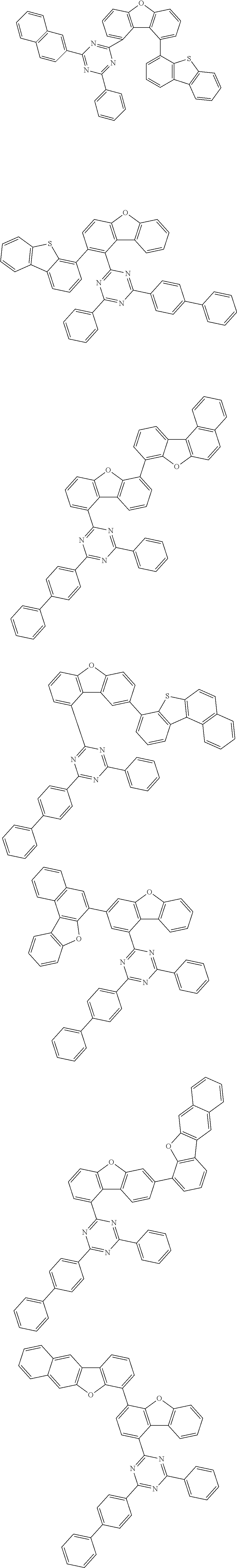

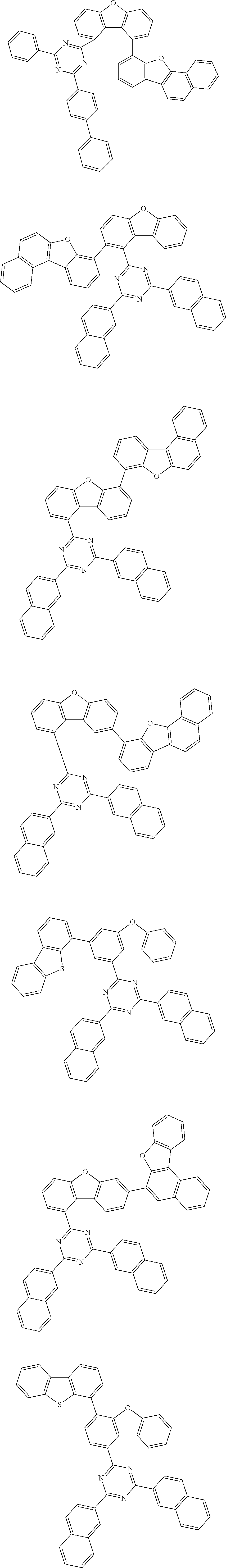

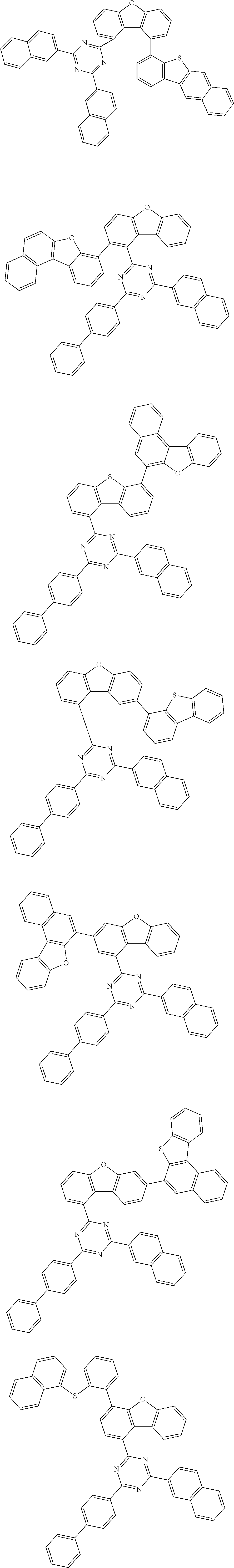

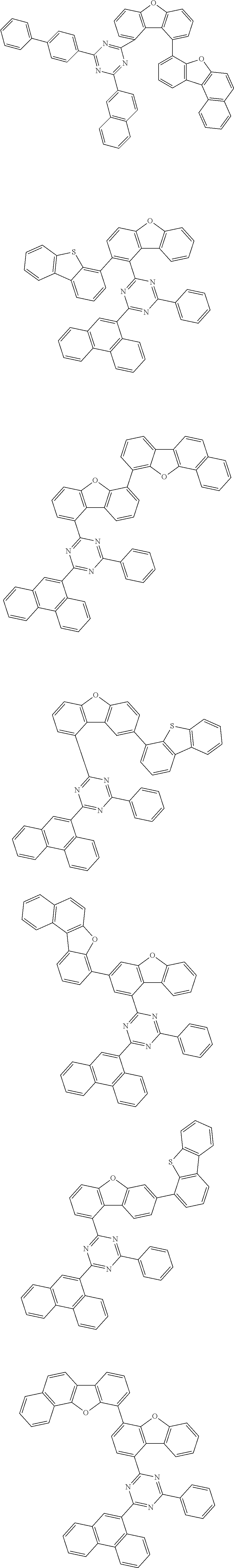

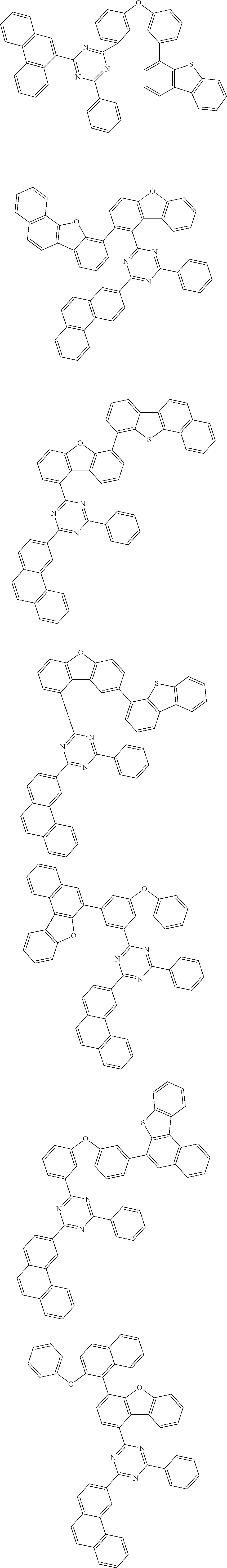

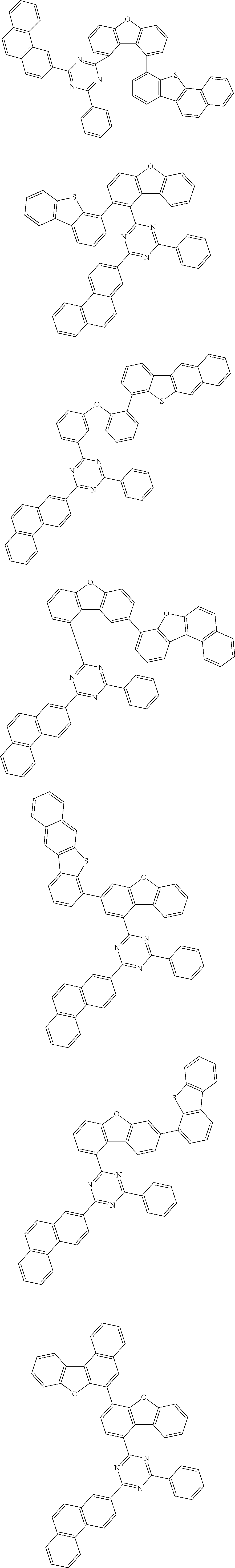

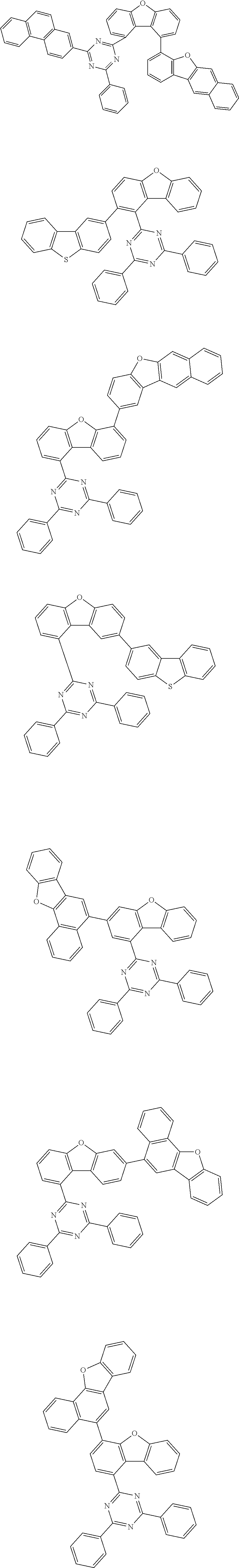

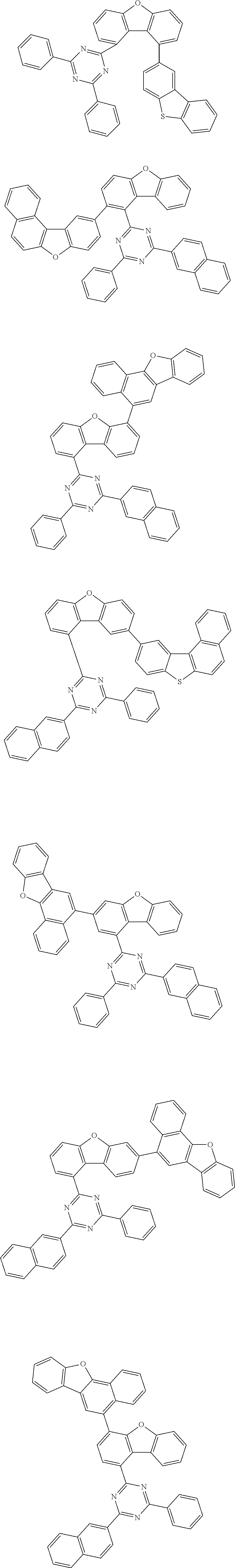

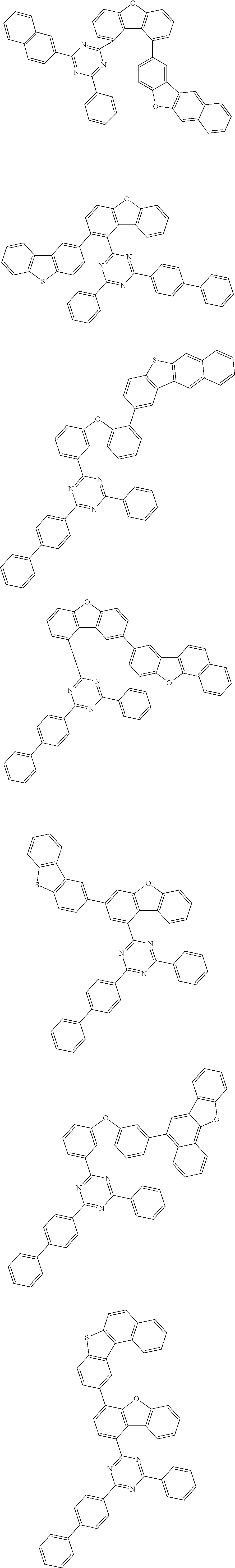

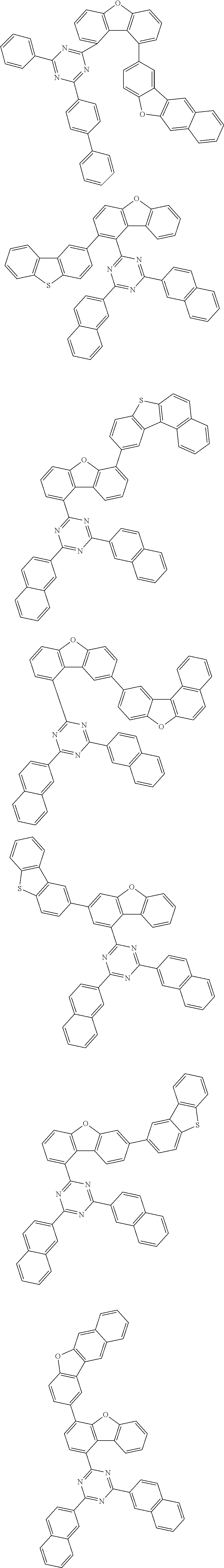

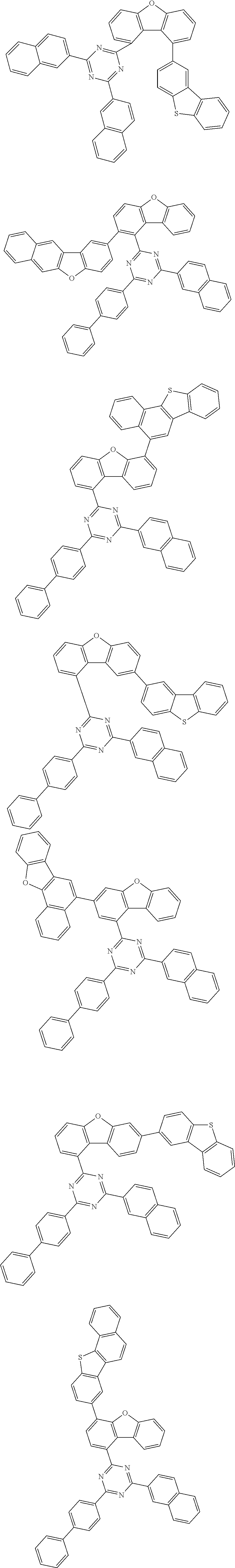

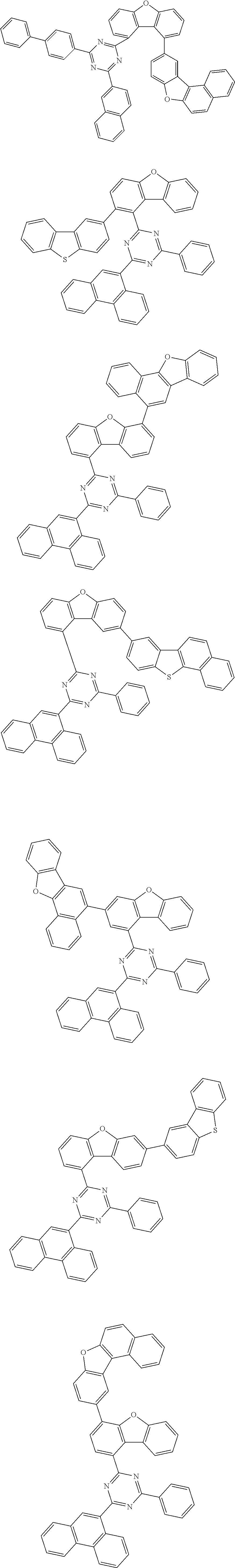

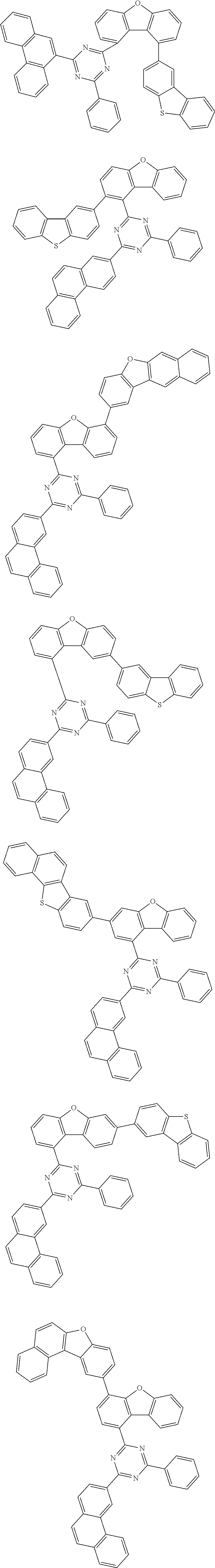

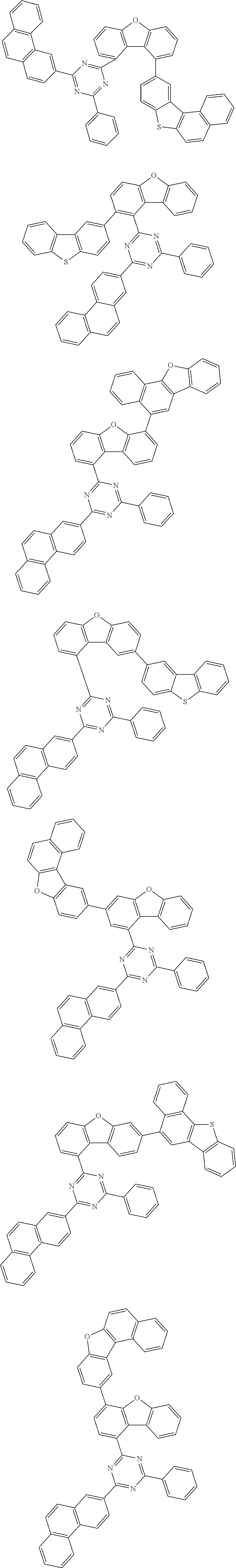

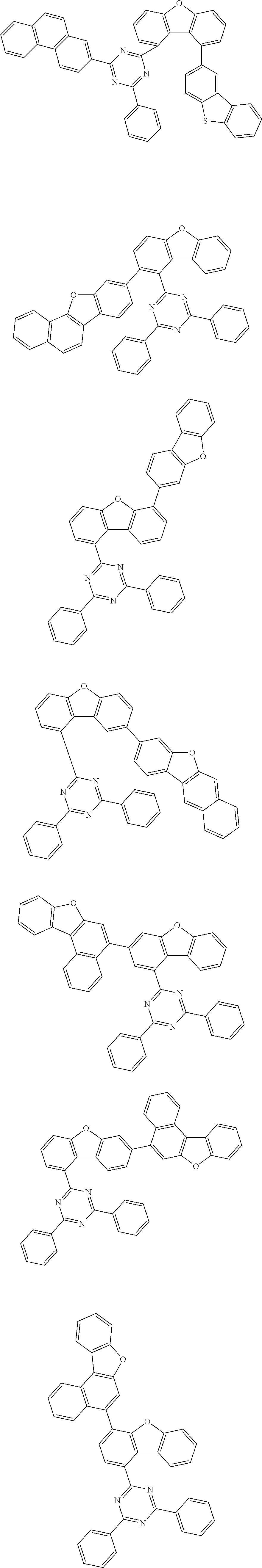

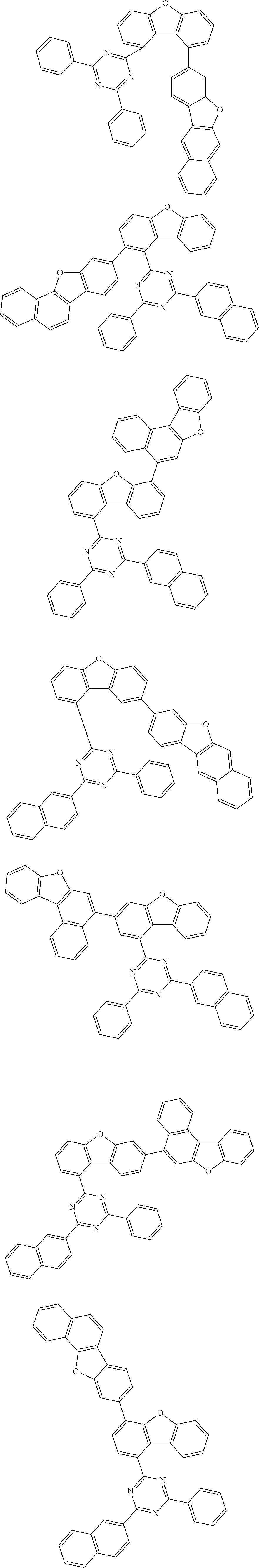

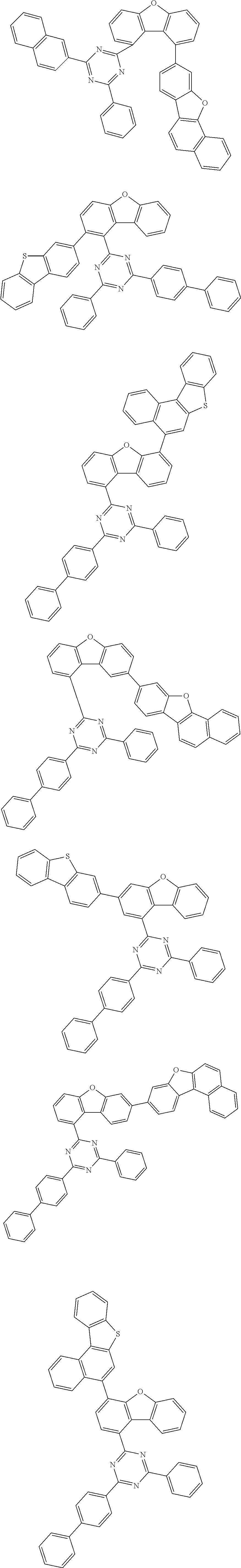

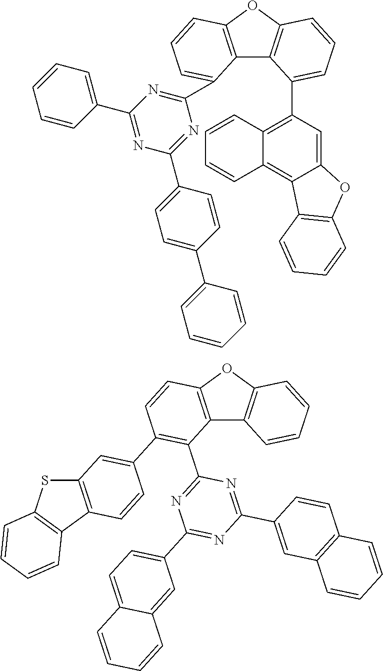

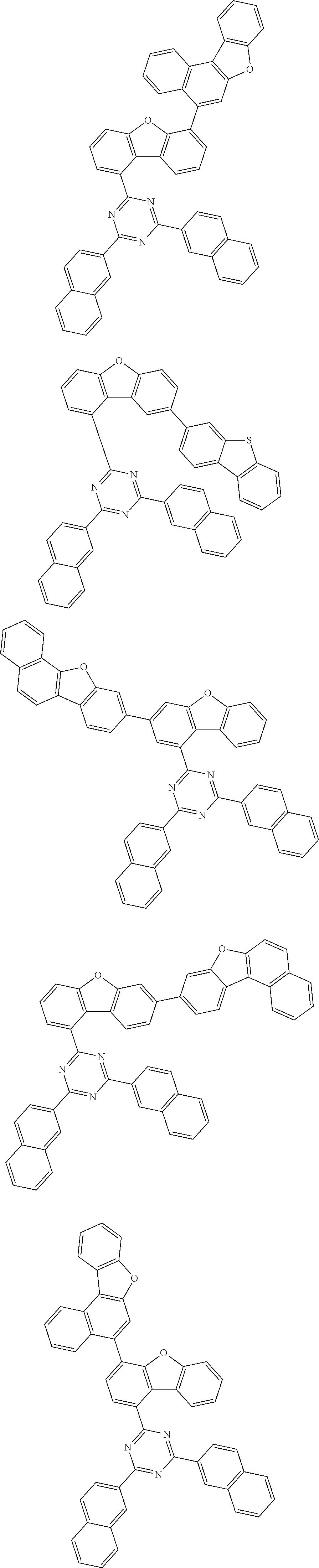

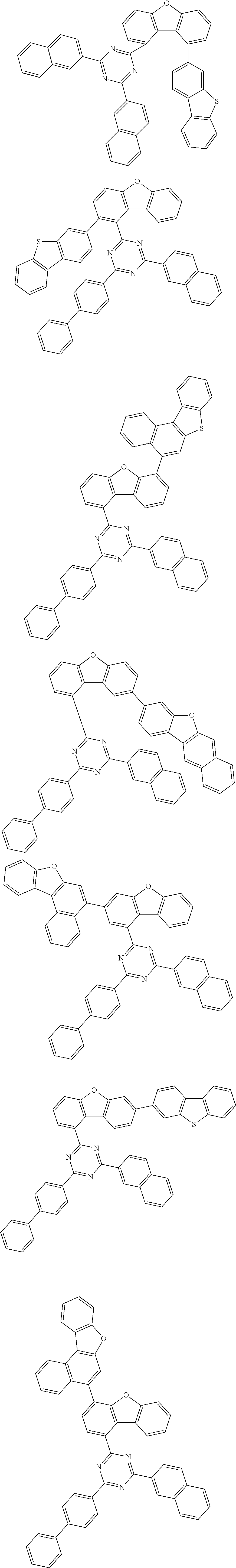

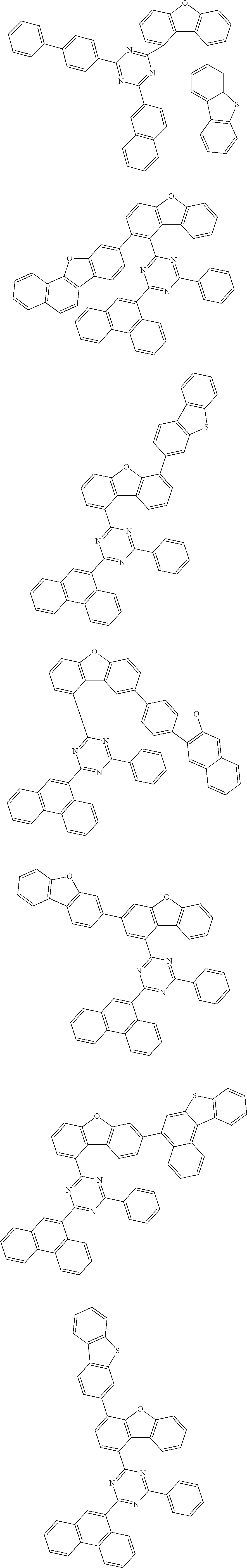

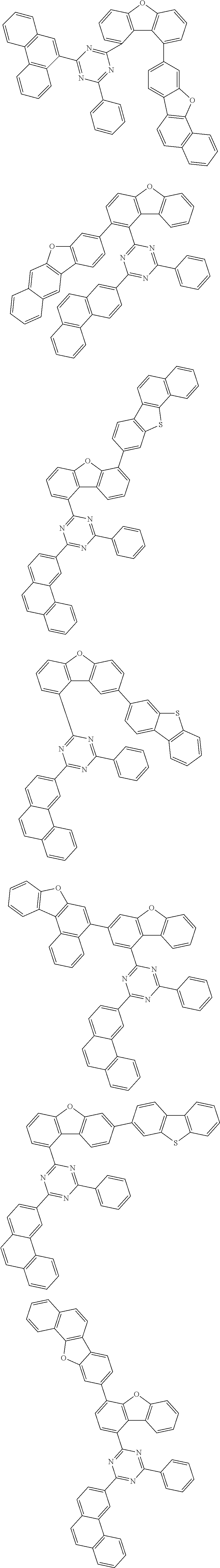

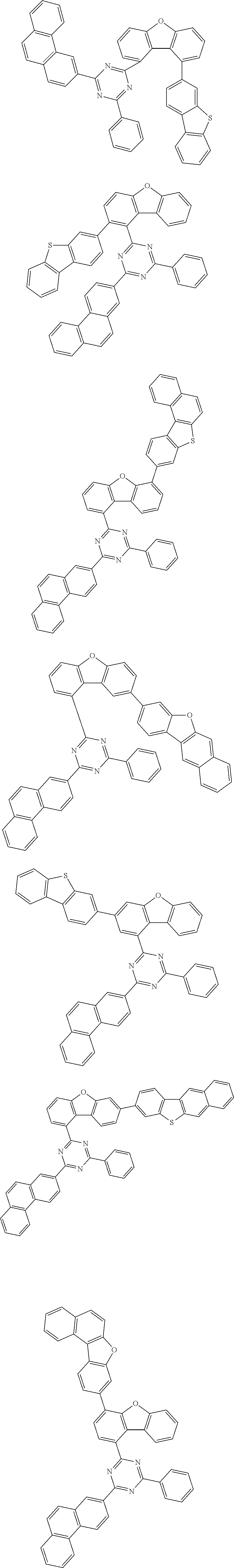

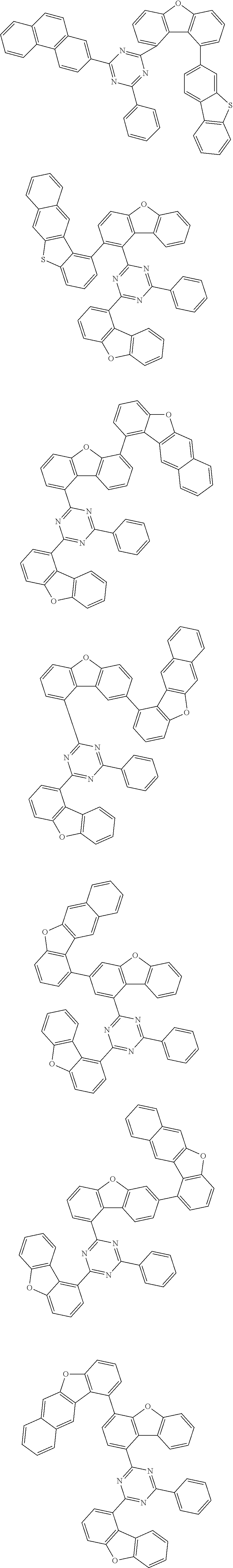

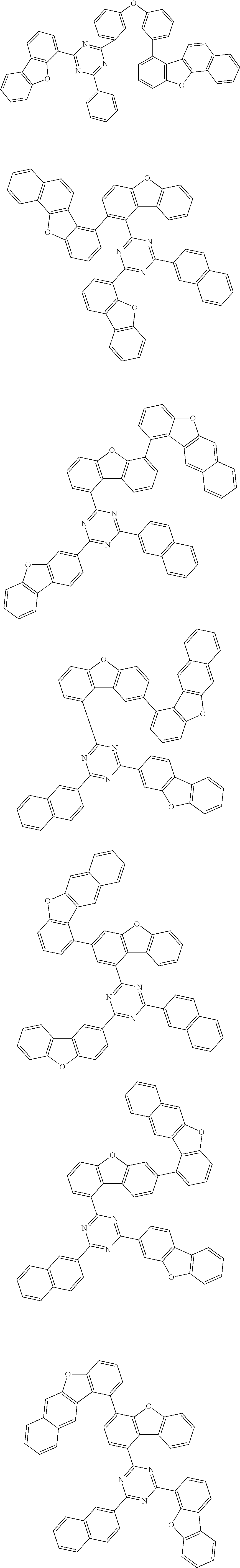

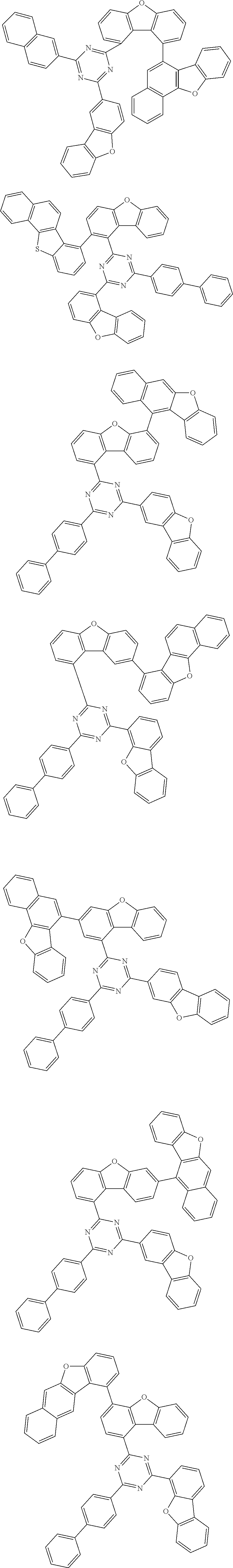

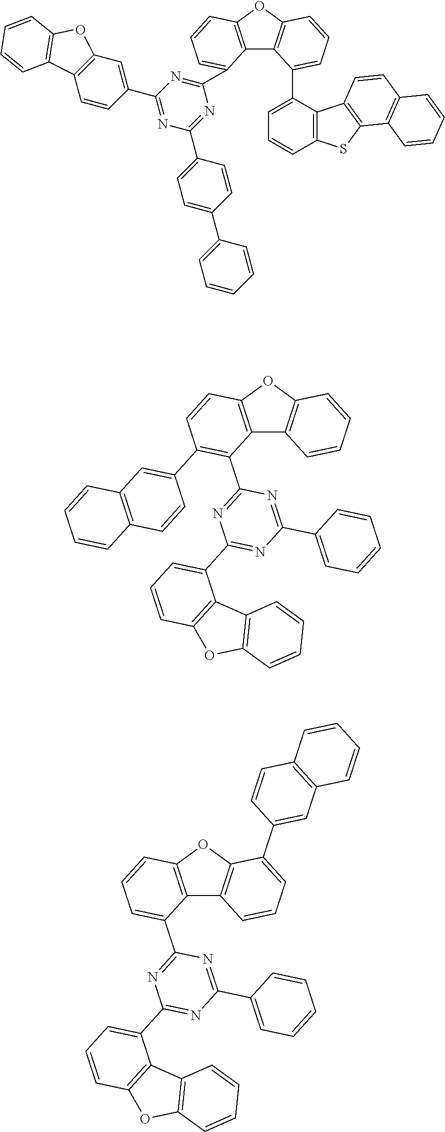 [SMILES: c1ccc(-c2ccc(-c3nc(-c4ccc5c(c4)oc4ccccc45)nc(-c4cccc5oc6cccc(-c7cccc8sc9c%10ccccc%10ccc9c78)c6c45)n3)cc2)cc1 ; c1ccc(-c2nc(-c3cccc4oc5ccccc5c34)nc(-c3c(-c4ccc5ccccc5c4)ccc4oc5ccccc5c34)n2)cc1 ; c1ccc(-c2nc(-c3cccc4oc5ccccc5c34)nc(-c3cccc4oc5c(-c6ccc7ccccc7c6)cccc5c34)n2)cc1]

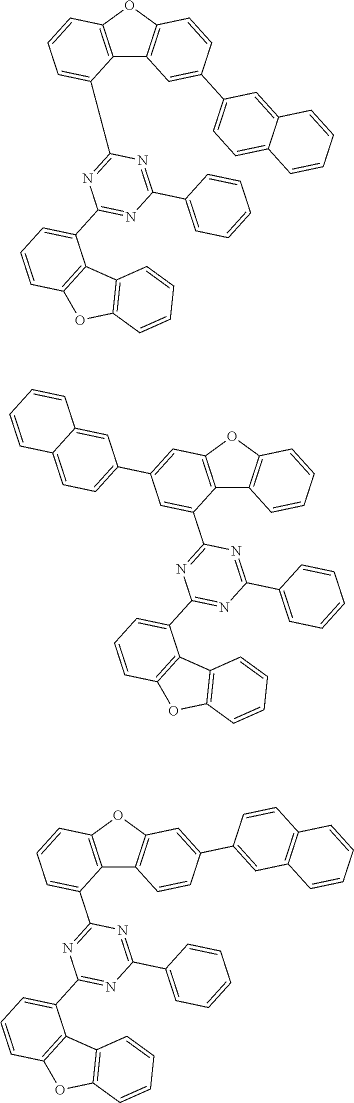 [SMILES: c1ccc(-c2nc(-c3cccc4oc5ccccc5c34)nc(-c3cccc4oc5ccc(-c6ccc7ccccc7c6)cc5c34)n2)cc1 ; c1ccc(-c2nc(-c3cccc4oc5ccccc5c34)nc(-c3cc(-c4ccc5ccccc5c4)cc4oc5ccccc5c34)n2)cc1 ; c1ccc(-c2nc(-c3cccc4oc5ccccc5c34)nc(-c3cccc4oc5cc(-c6ccc7ccccc7c6)ccc5c34)n2)cc1]

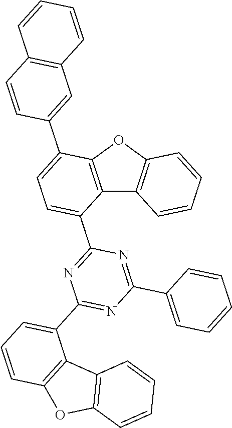 [SMILES: c1ccc(-c2nc(-c3cccc4oc5ccccc5c34)nc(-c3ccc(-c4ccc5ccccc5c4)c4oc5ccccc5c34)n2)cc1]

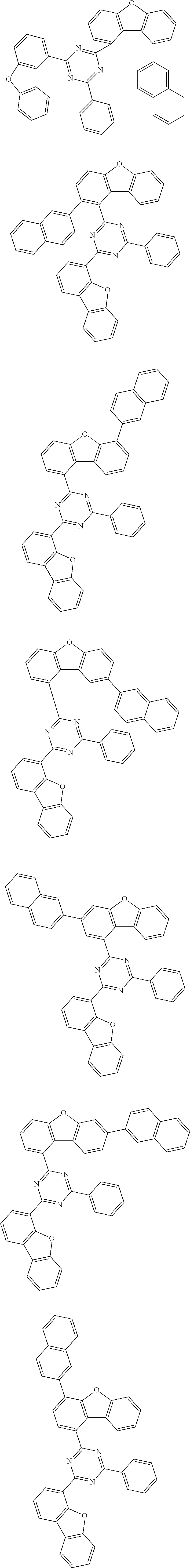

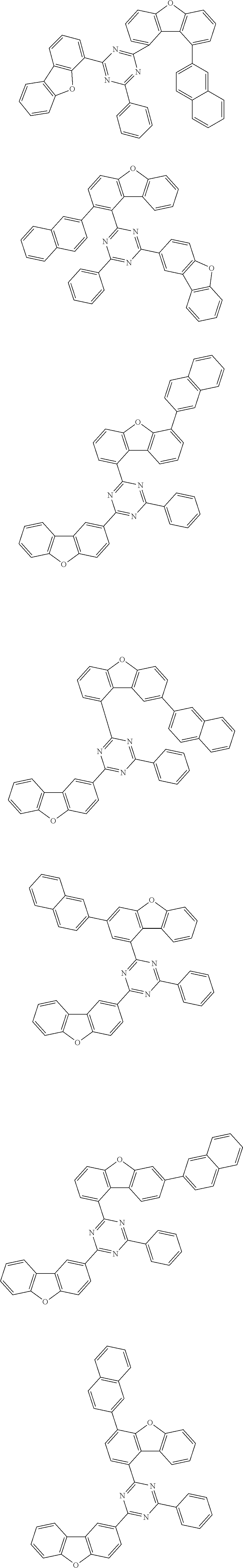

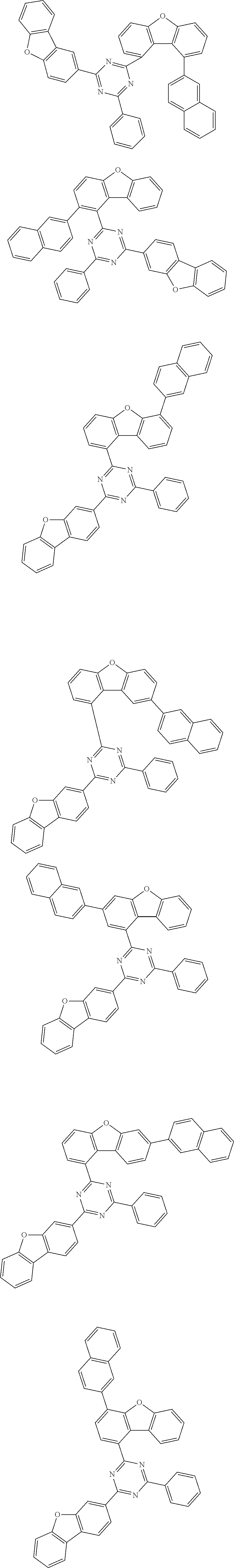

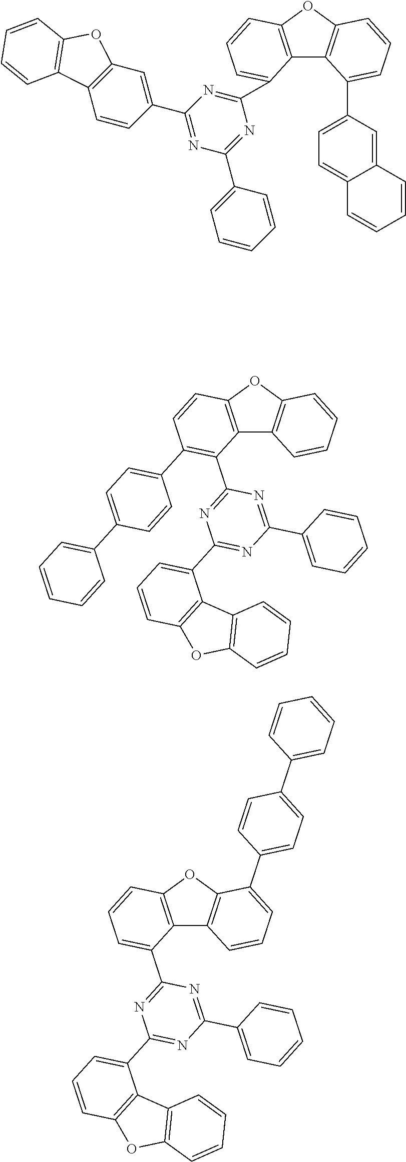

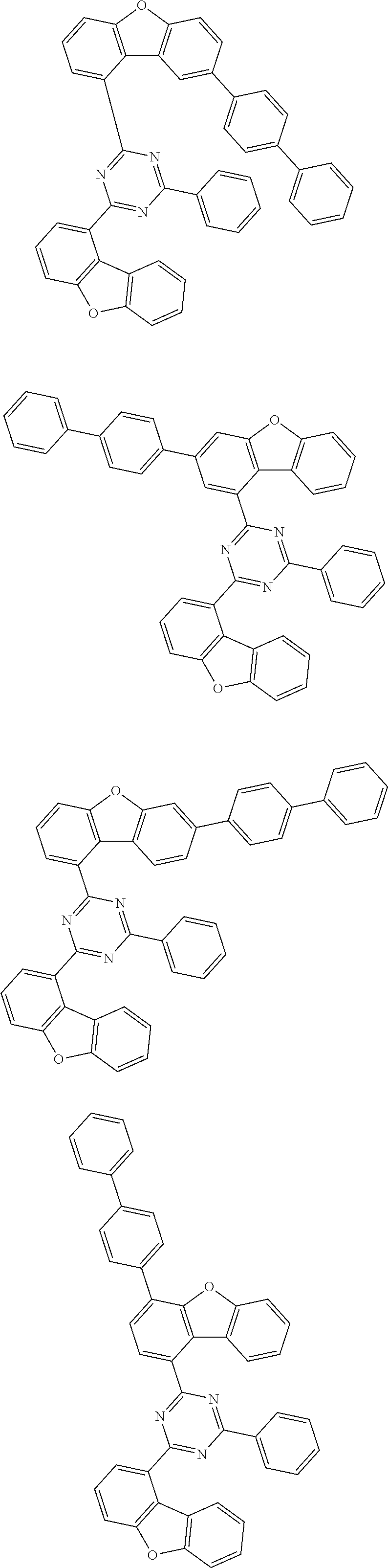 [SMILES: c1ccc(-c2ccc(-c3ccc4oc5cccc(-c6nc(-c7ccccc7)nc(-c7cccc8oc9ccccc9c78)n6)c5c4c3)cc2)cc1 ; c1ccc(-c2ccc(-c3cc(-c4nc(-c5ccccc5)nc(-c5cccc6oc7ccccc7c56)n4)c4c(c3)oc3ccccc34)cc2)cc1 ; c1ccc(-c2ccc(-c3ccc4c(c3)oc3cccc(-c5nc(-c6ccccc6)nc(-c6cccc7oc8ccccc8c67)n5)c34)cc2)cc1 ; c1ccc(-c2ccc(-c3ccc(-c4nc(-c5ccccc5)nc(-c5cccc6oc7ccccc7c56)n4)c4c3oc3ccccc34)cc2)cc1]

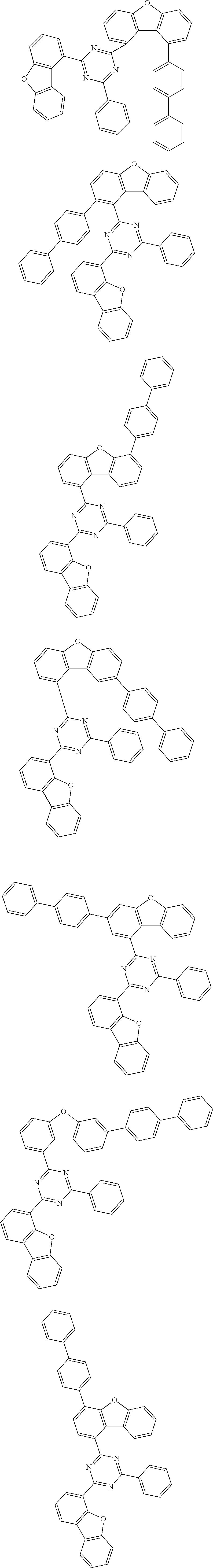

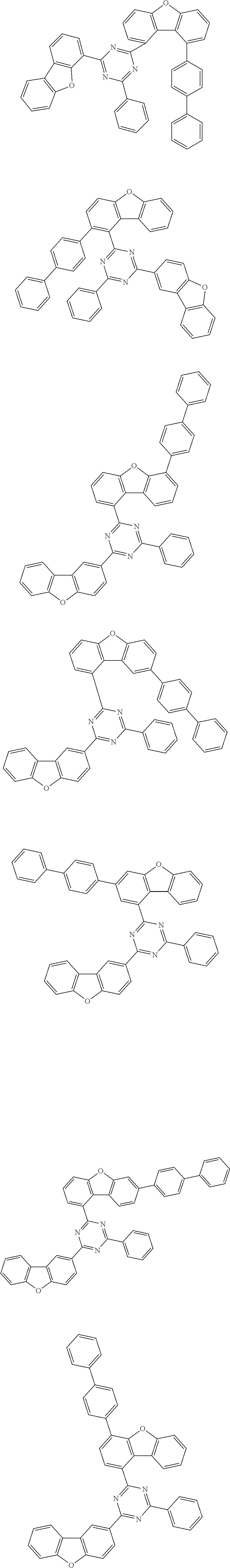

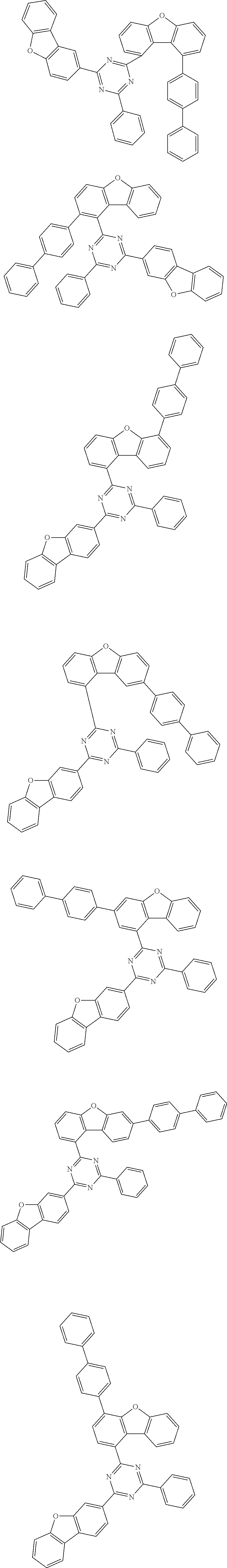

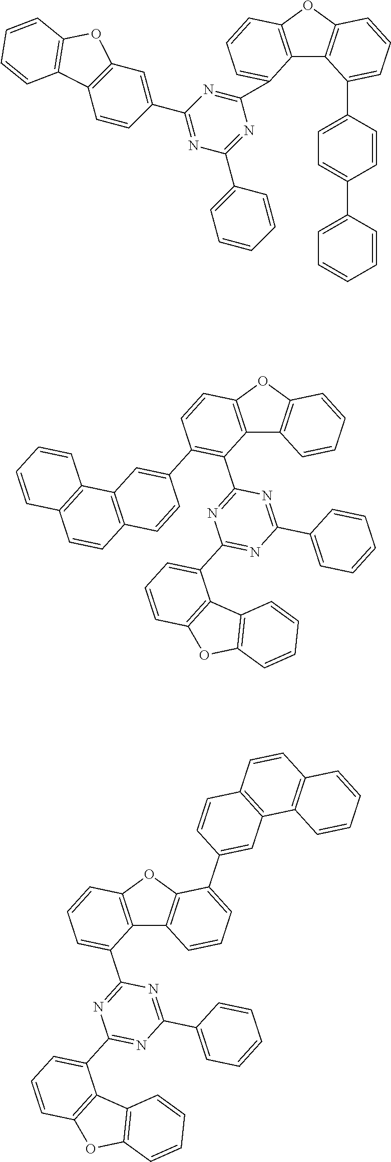 [SMILES: c1ccc(-c2ccc(-c3cccc4oc5cccc(-c6nc(-c7ccccc7)nc(-c7ccc8c(c7)oc7ccccc78)n6)c5c34)cc2)cc1 ; c1ccc(-c2nc(-c3cccc4oc5ccccc5c34)nc(-c3c(-c4ccc5ccc6ccccc6c5c4)ccc4oc5ccccc5c34)n2)cc1 ; c1ccc(-c2nc(-c3cccc4oc5ccccc5c34)nc(-c3cccc4oc5c(-c6ccc7ccc8ccccc8c7c6)cccc5c34)n2)cc1]

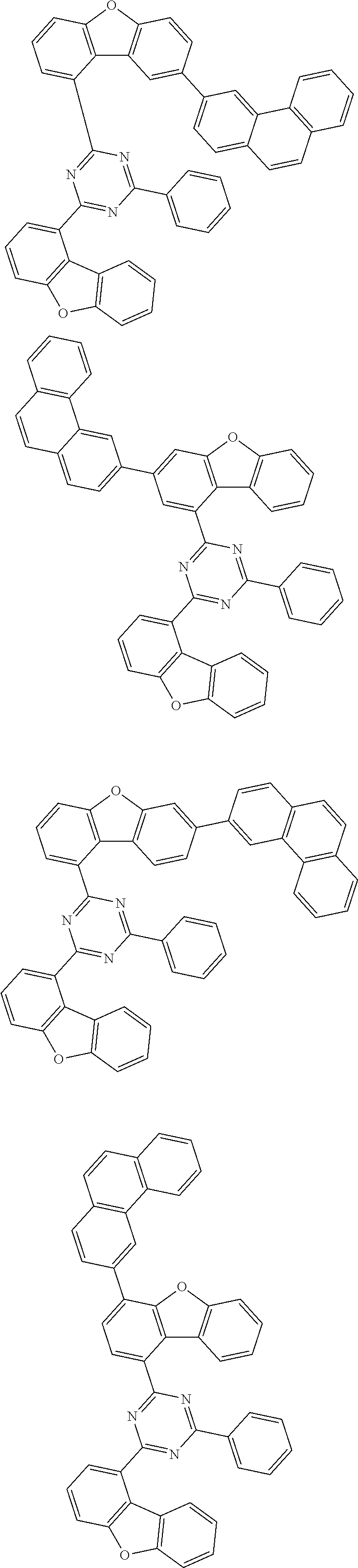

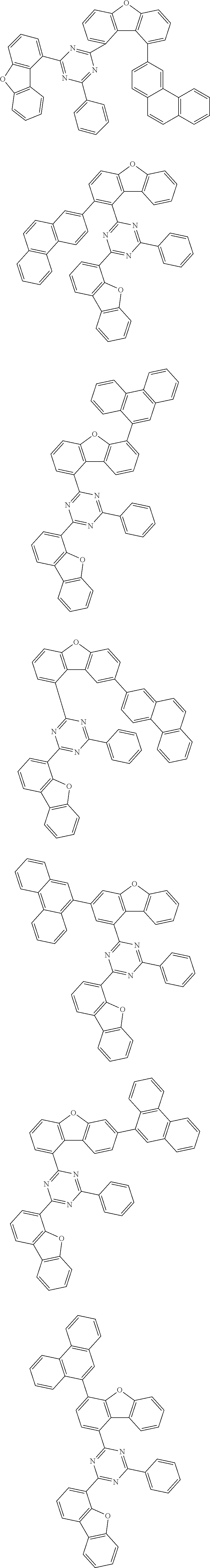

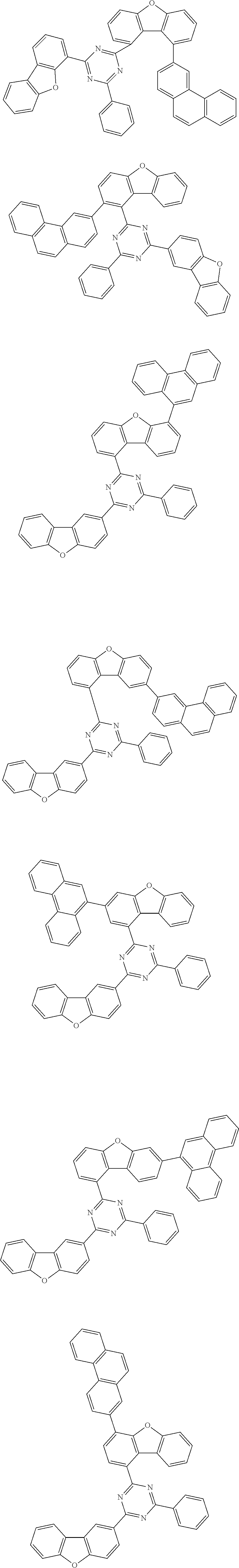

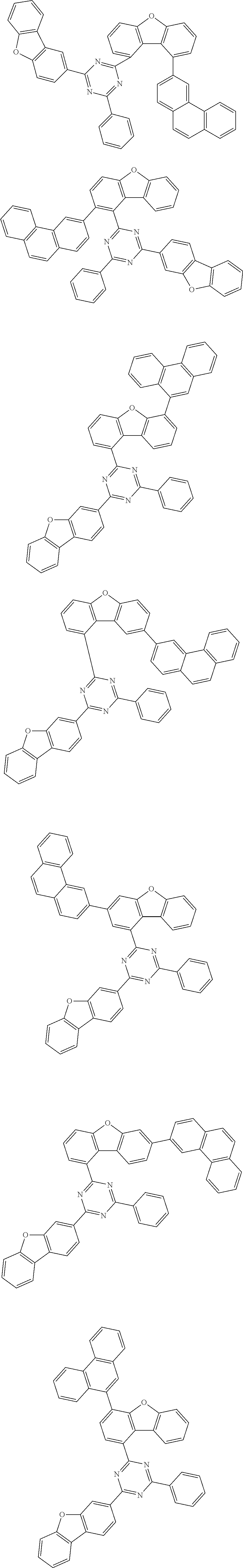

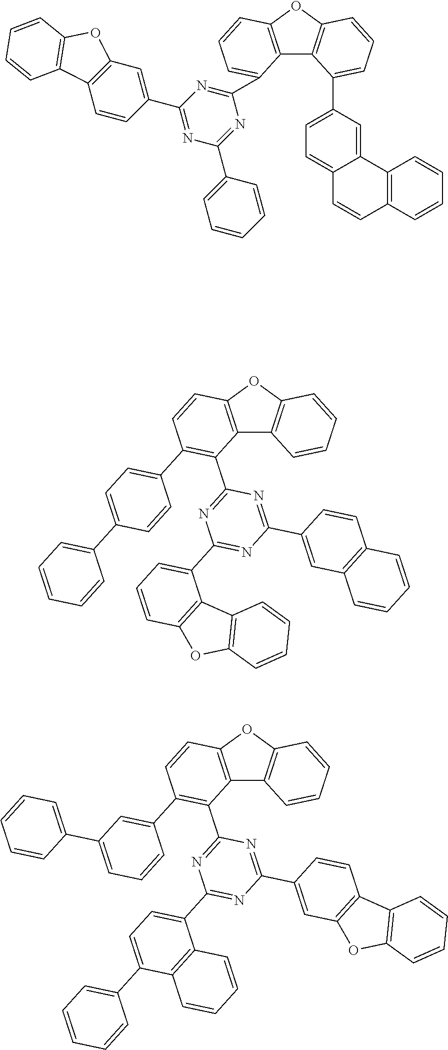 [SMILES: c1ccc(-c2nc(-c3ccc4c(c3)oc3ccccc34)nc(-c3cccc4oc5cccc(-c6ccc7ccc8ccccc8c7c6)c5c34)n2)cc1 ; c1ccc(-c2ccc(-c3ccc4oc5ccccc5c4c3-c3nc(-c4ccc5ccccc5c4)nc(-c4cccc5oc6ccccc6c45)n3)cc2)cc1 ; c1ccc(-c2cccc(-c3ccc4oc5ccccc5c4c3-c3nc(-c4ccc5c(c4)oc4ccccc45)nc(-c4ccc(-c5ccccc5)c5ccccc45)n3)c2)cc1]

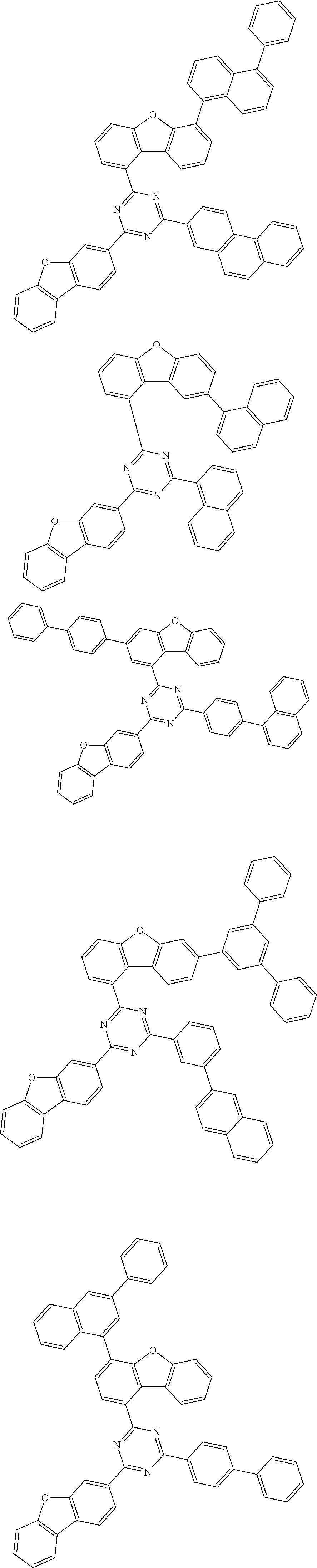

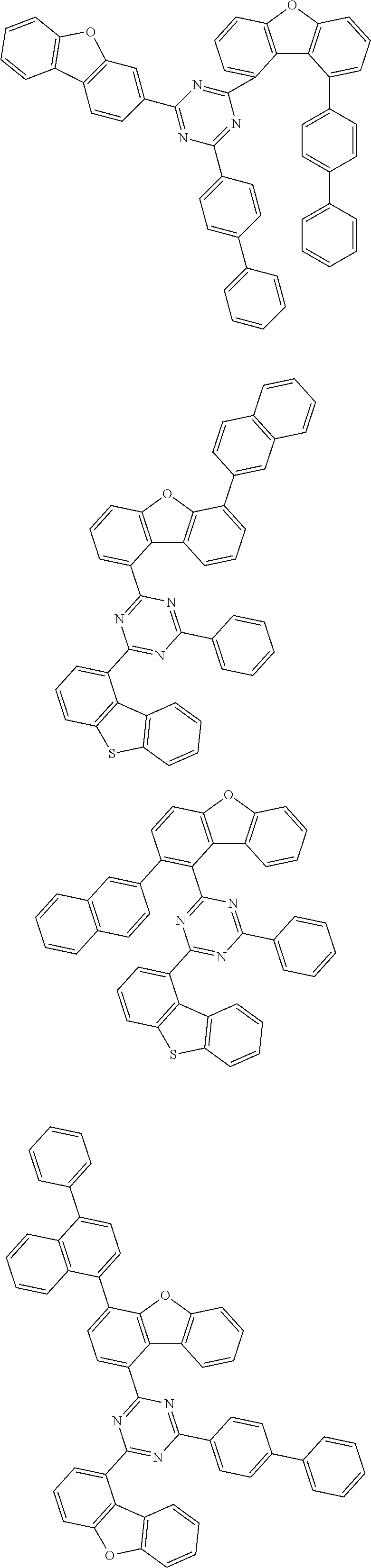

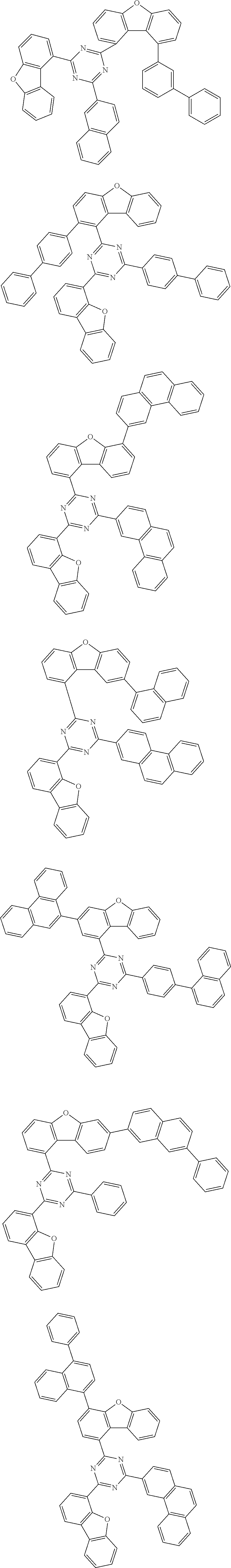

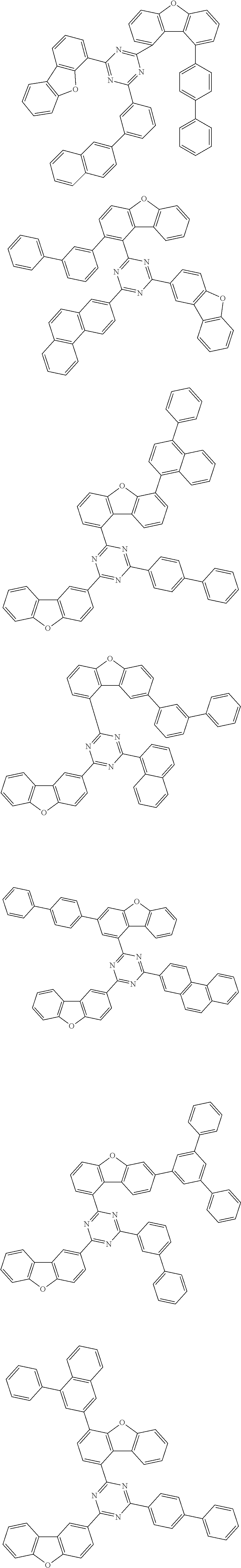

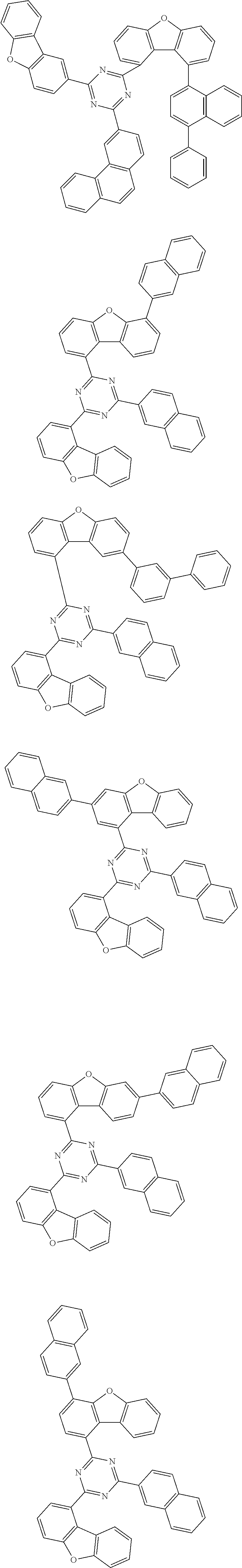

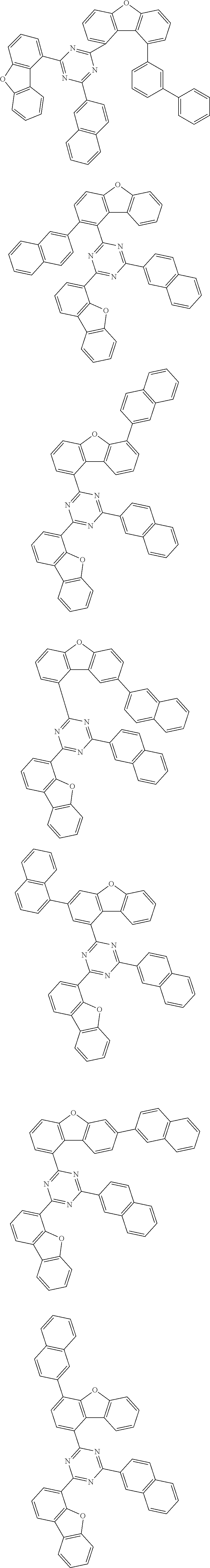

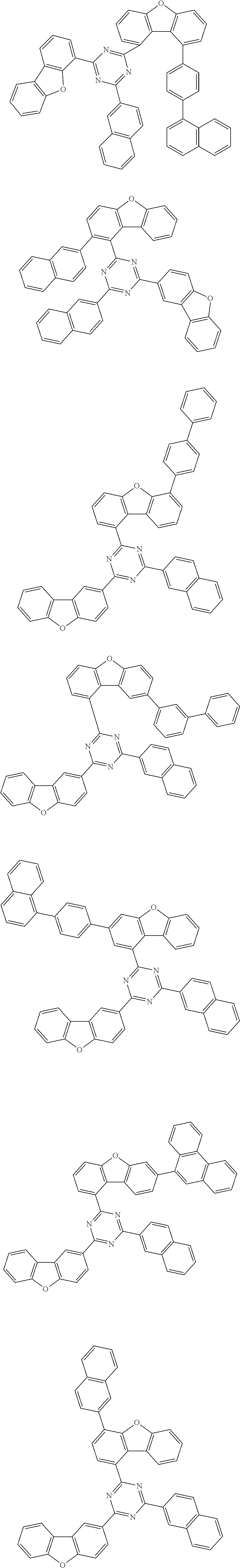

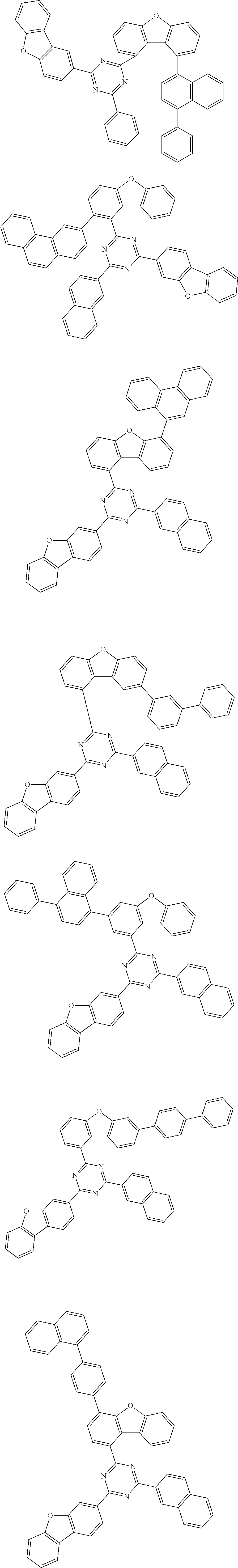

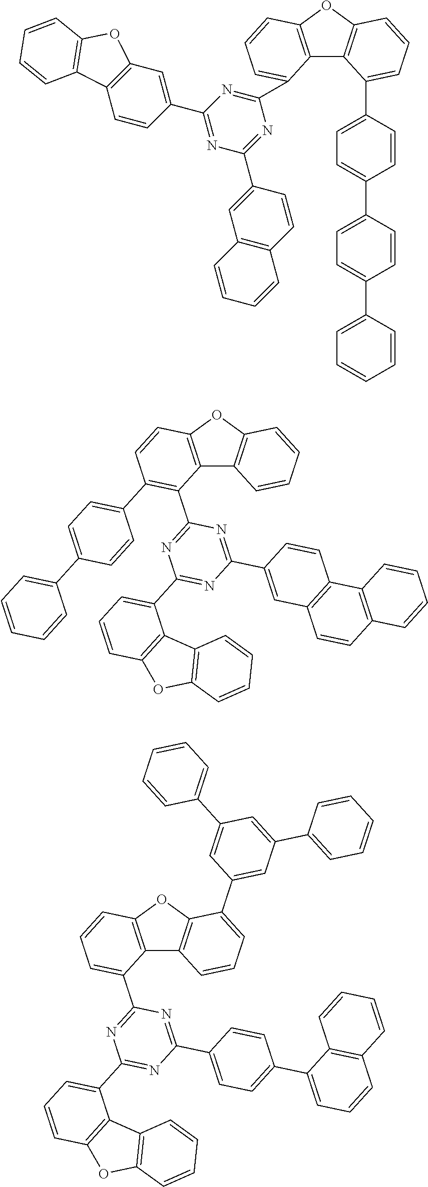 [SMILES: c1ccc(-c2ccc(-c3ccc(-c4cccc5oc6cccc(-c7nc(-c8ccc9ccccc9c8)nc(-c8ccc9c(c8)oc8ccccc89)n7)c6c45)cc3)cc2)cc1 ; c1ccc(-c2ccc(-c3ccc4oc5ccccc5c4c3-c3nc(-c4ccc5c(ccc6ccccc65)c4)nc(-c4cccc5oc6ccccc6c45)n3)cc2)cc1 ; c1ccc(-c2cc(-c3ccccc3)cc(-c3cccc4c3oc3cccc(-c5nc(-c6ccc(-c7cccc8ccccc78)cc6)nc(-c6cccc7oc8ccccc8c67)n5)c34)c2)cc1]

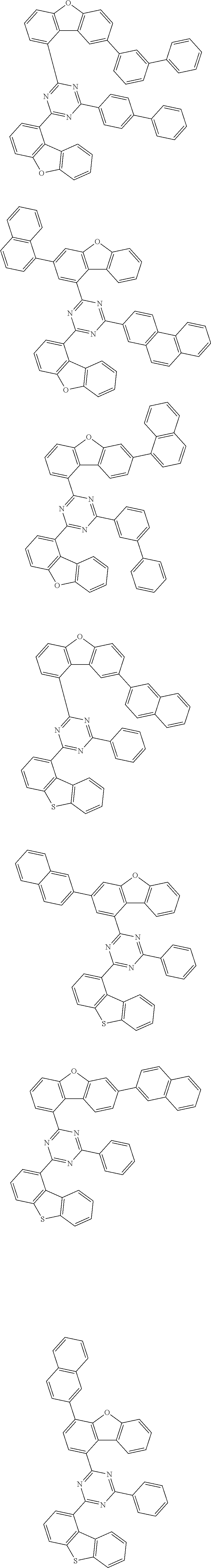

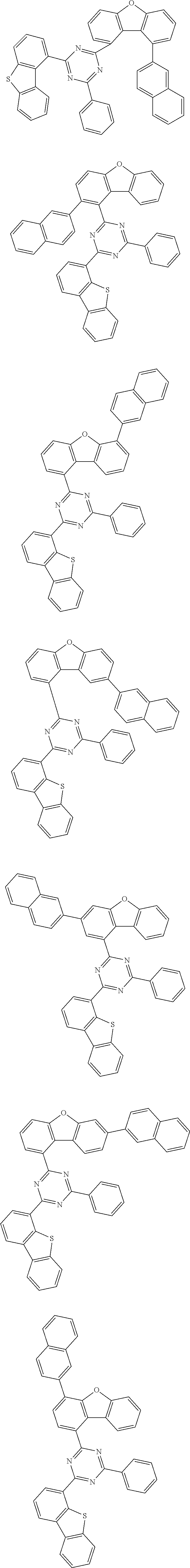

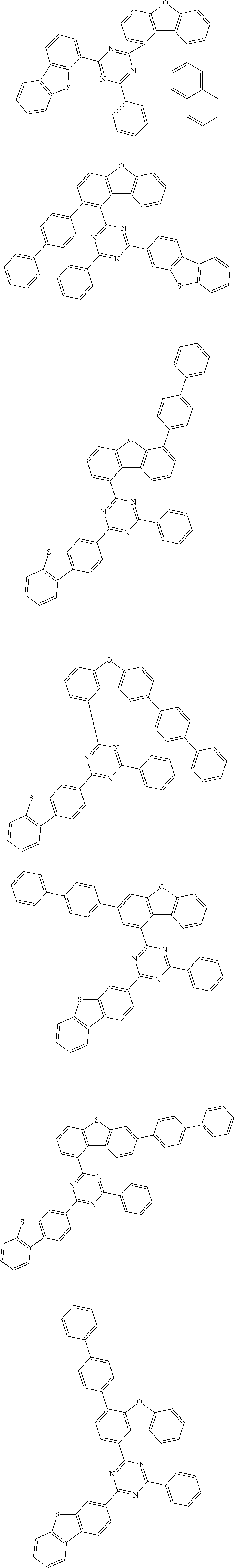

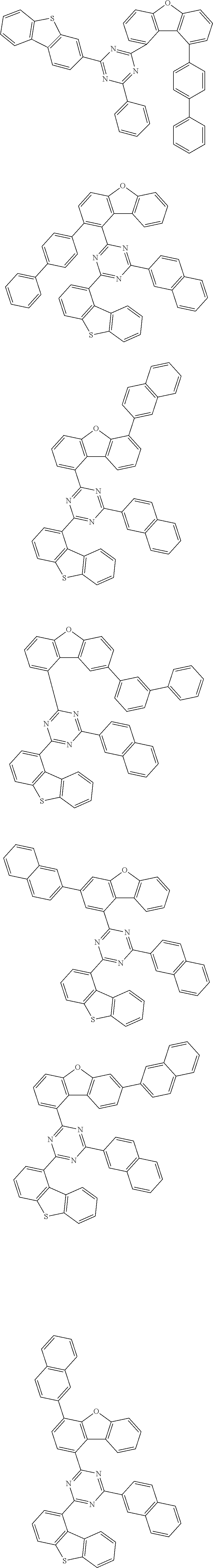

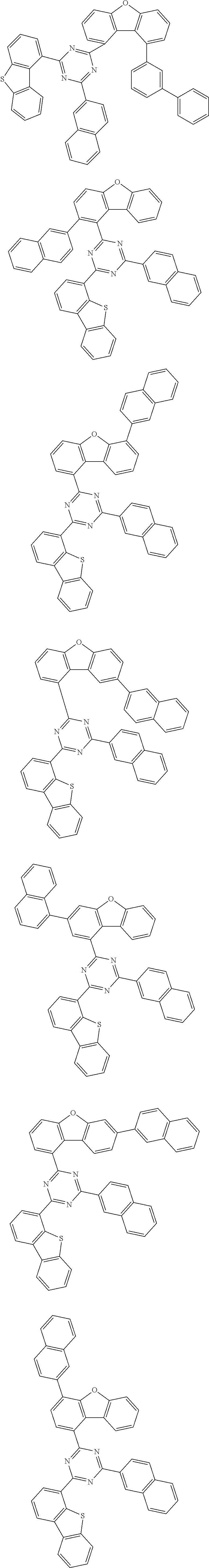

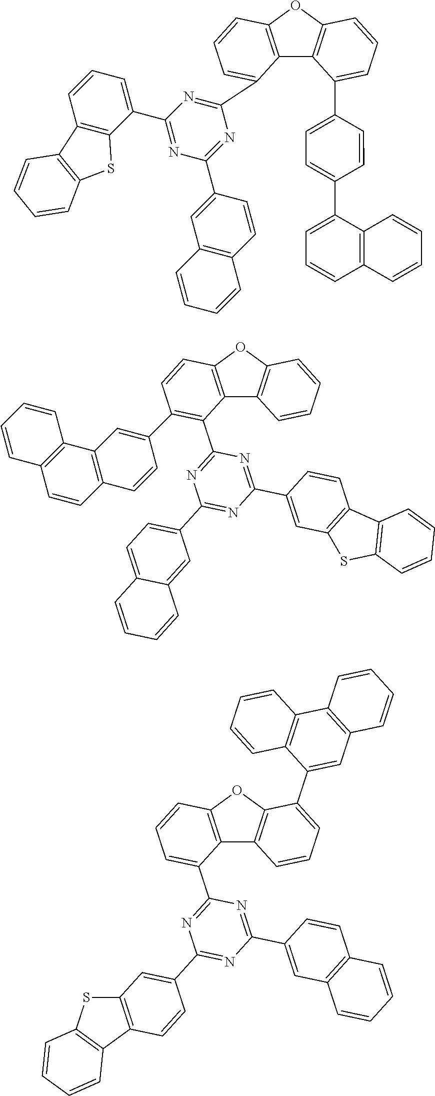

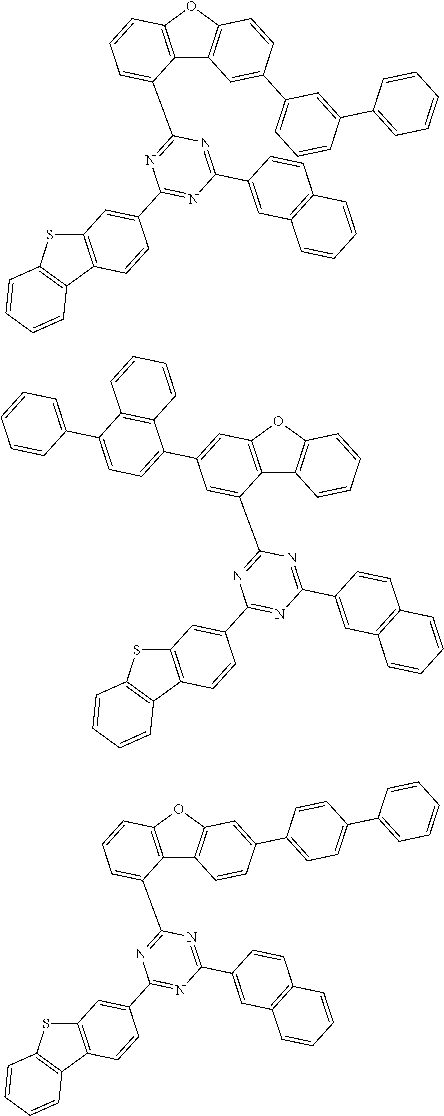 [SMILES: c1ccc(-c2cccc(-c3ccc4oc5cccc(-c6nc(-c7ccc8ccccc8c7)nc(-c7ccc8c(c7)sc7ccccc78)n6)c5c4c3)c2)cc1 ; c1ccc(-c2ccc(-c3cc(-c4nc(-c5ccc6ccccc6c5)nc(-c5ccc6c(c5)sc5ccccc56)n4)c4c(c3)oc3ccccc34)c3ccccc23)cc1 ; c1ccc(-c2ccc(-c3ccc4c(c3)oc3cccc(-c5nc(-c6ccc7ccccc7c6)nc(-c6ccc7c(c6)sc6ccccc67)n5)c34)cc2)cc1]

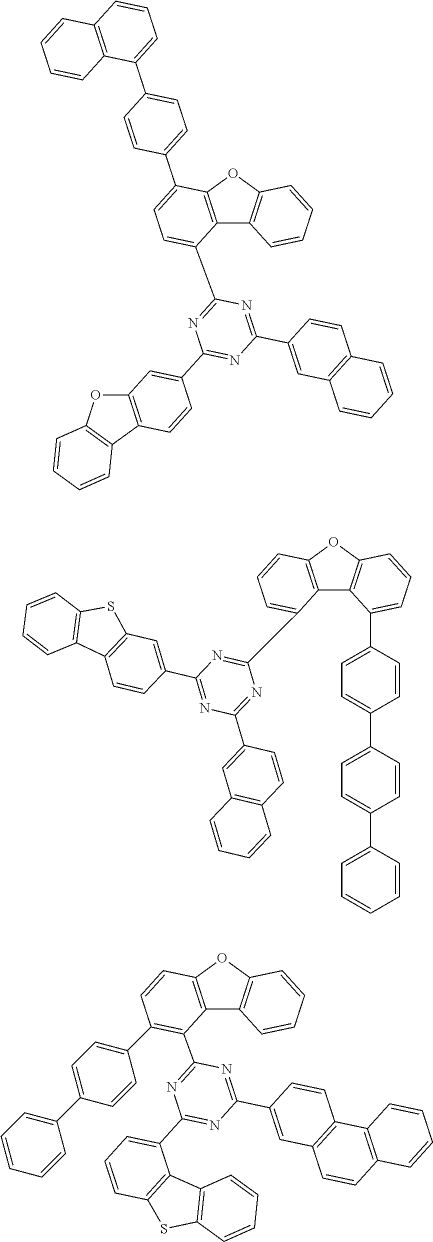 [SMILES: c1ccc2cc(-c3nc(-c4ccc5c(c4)oc4ccccc45)nc(-c4ccc(-c5ccc(-c6cccc7ccccc67)cc5)c5oc6ccccc6c45)n3)ccc2c1 ; c1ccc(-c2ccc(-c3ccc(-c4cccc5oc6cccc(-c7nc(-c8ccc9ccccc9c8)nc(-c8ccc9c(c8)sc8ccccc89)n7)c6c45)cc3)cc2)cc1 ; c1ccc(-c2ccc(-c3ccc4oc5ccccc5c4c3-c3nc(-c4ccc5c(ccc6ccccc65)c4)nc(-c4cccc5sc6ccccc6c45)n3)cc2)cc1]

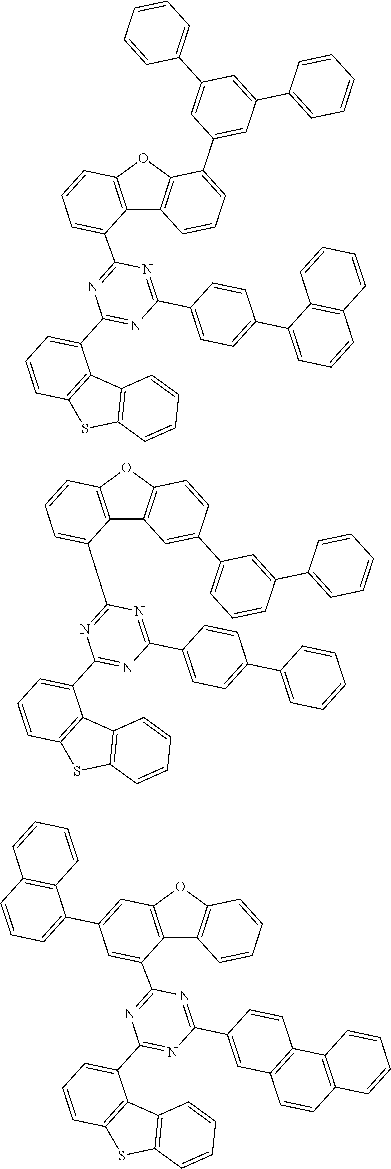

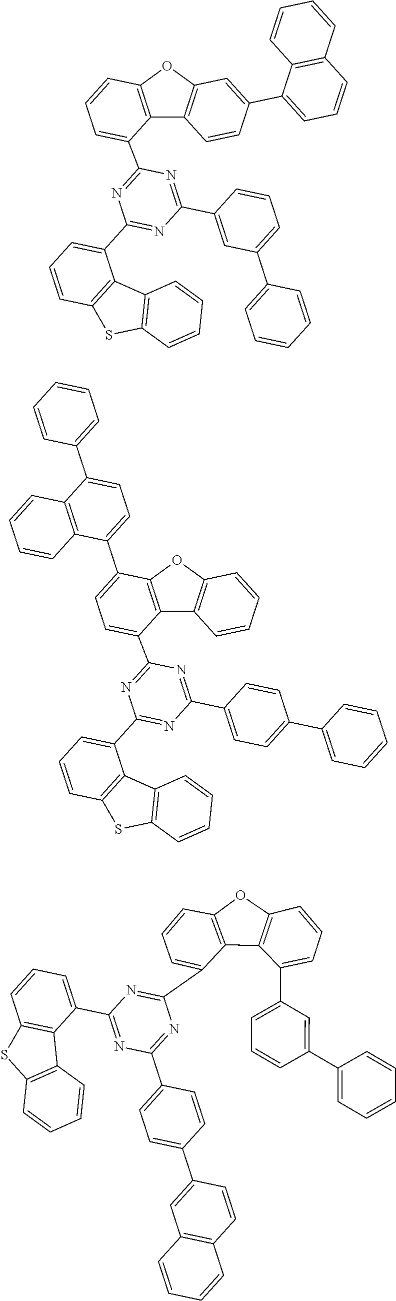 [SMILES: c1ccc(-c2cccc(-c3nc(-c4cccc5oc6cc(-c7cccc8ccccc78)ccc6c45)nc(-c4cccc5sc6ccccc6c45)n3)c2)cc1 ; c1ccc(-c2ccc(-c3nc(-c4cccc5sc6ccccc6c45)nc(-c4ccc(-c5ccc(-c6ccccc6)c6ccccc56)c5oc6ccccc6c45)n3)cc2)cc1 ; c1ccc(-c2cccc(-c3cccc4oc5cccc(-c6nc(-c7ccc(-c8ccc9ccccc9c8)cc7)nc(-c7cccc8sc9ccccc9c78)n6)c5c34)c2)cc1]

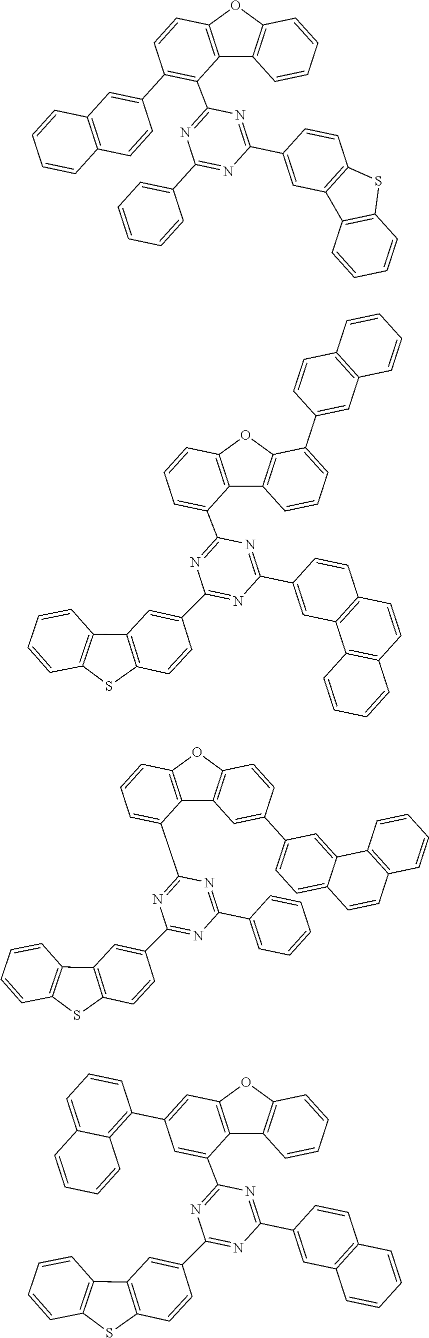

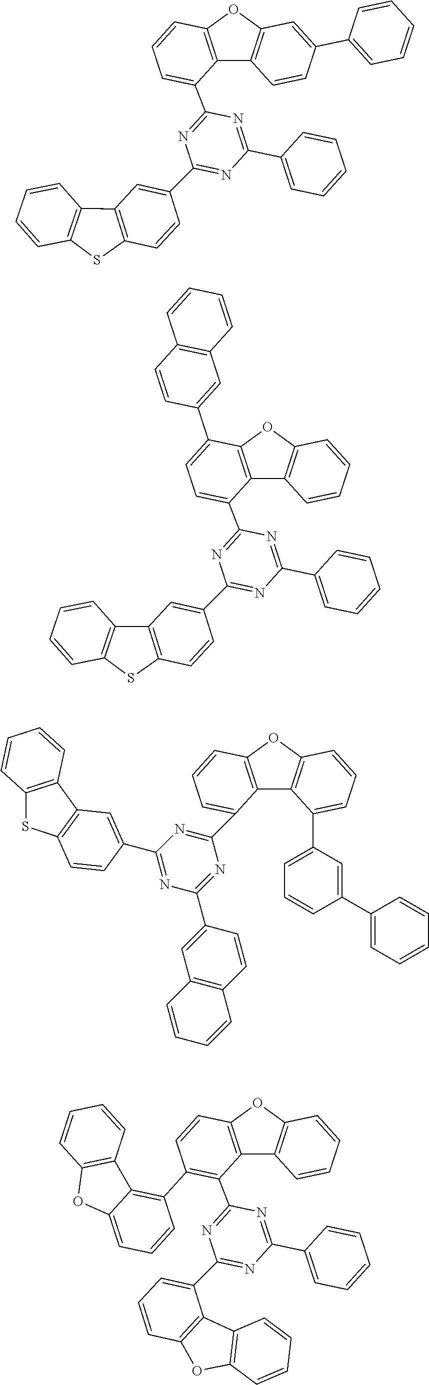 [SMILES: c1ccc(-c2ccc3c(c2)oc2cccc(-c4nc(-c5ccccc5)nc(-c5ccc6sc7ccccc7c6c5)n4)c23)cc1 ; c1ccc(-c2nc(-c3ccc4sc5ccccc5c4c3)nc(-c3ccc(-c4ccc5ccccc5c4)c4oc5ccccc5c34)n2)cc1 ; c1ccc(-c2cccc(-c3cccc4oc5cccc(-c6nc(-c7ccc8ccccc8c7)nc(-c7ccc8sc9ccccc9c8c7)n6)c5c34)c2)cc1 ; c1ccc(-c2nc(-c3cccc4oc5ccccc5c34)nc(-c3c(-c4cccc5oc6ccccc6c45)ccc4oc5ccccc5c34)n2)cc1]

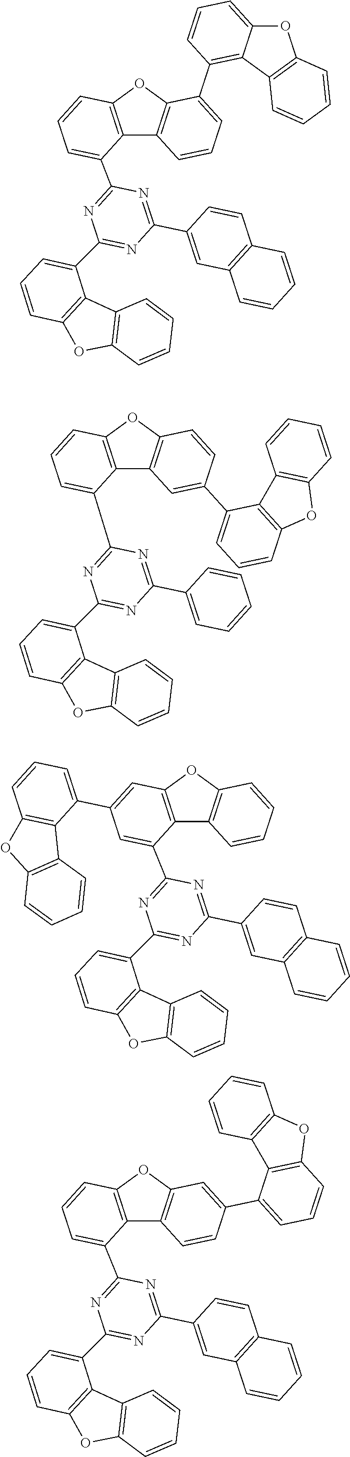

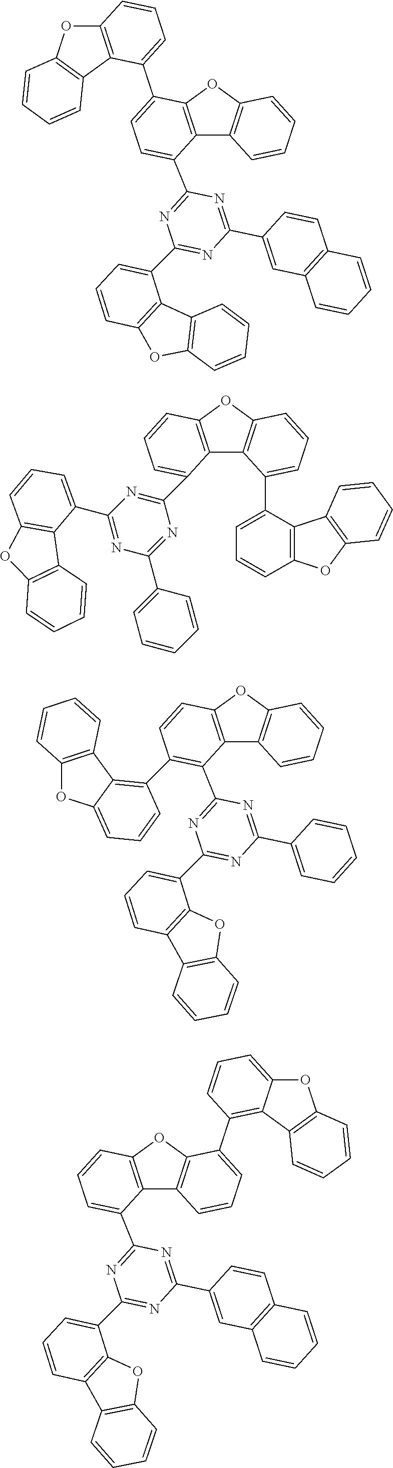

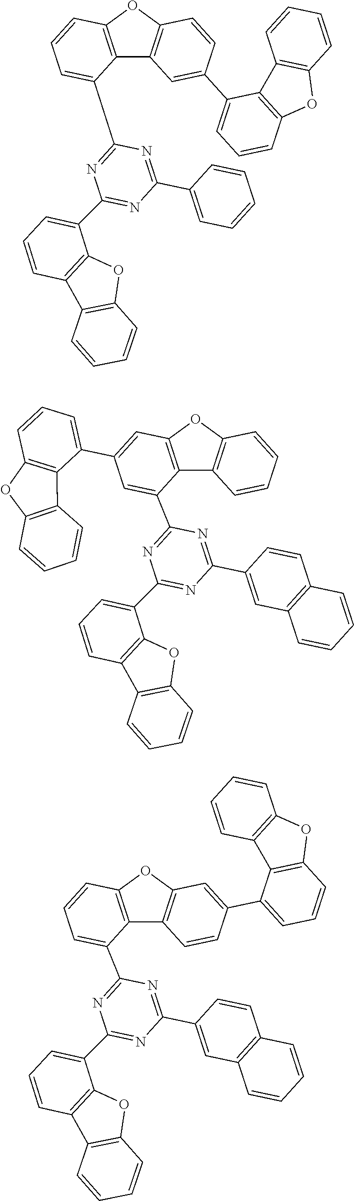 [SMILES: c1ccc(-c2nc(-c3cccc4c3oc3ccccc34)nc(-c3cccc4oc5ccc(-c6cccc7oc8ccccc8c67)cc5c34)n2)cc1 ; c1ccc2cc(-c3nc(-c4cccc5c4oc4ccccc45)nc(-c4cc(-c5cccc6oc7ccccc7c56)cc5oc6ccccc6c45)n3)ccc2c1 ; c1ccc2cc(-c3nc(-c4cccc5c4oc4ccccc45)nc(-c4cccc5oc6cc(-c7cccc8oc9ccccc9c78)ccc6c45)n3)ccc2c1]

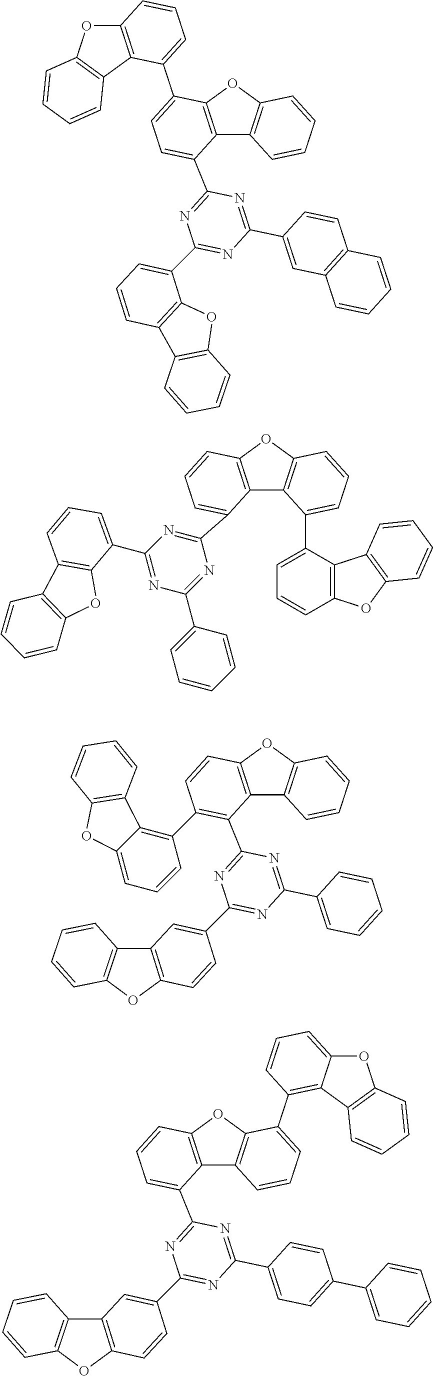

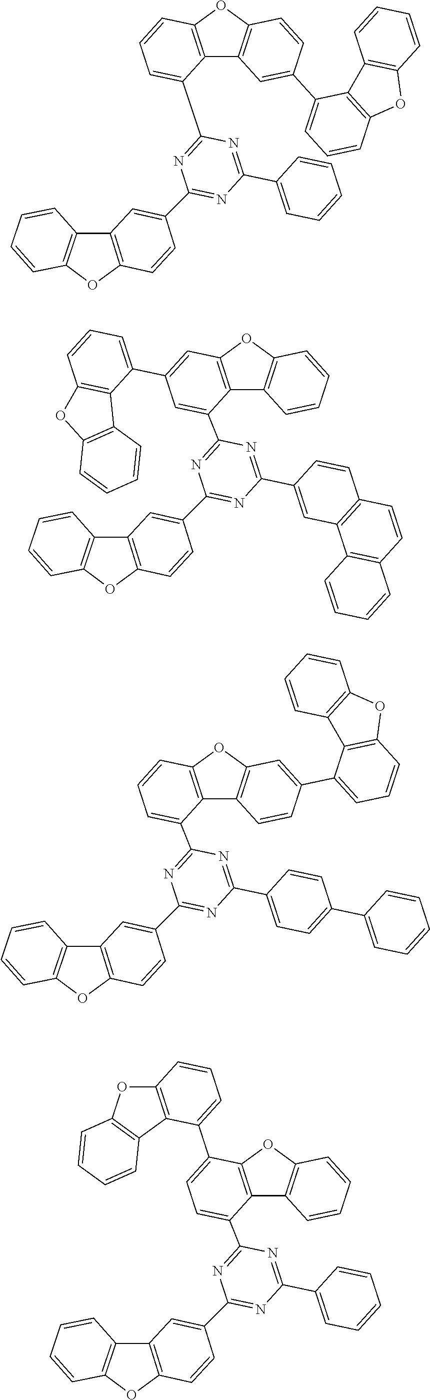

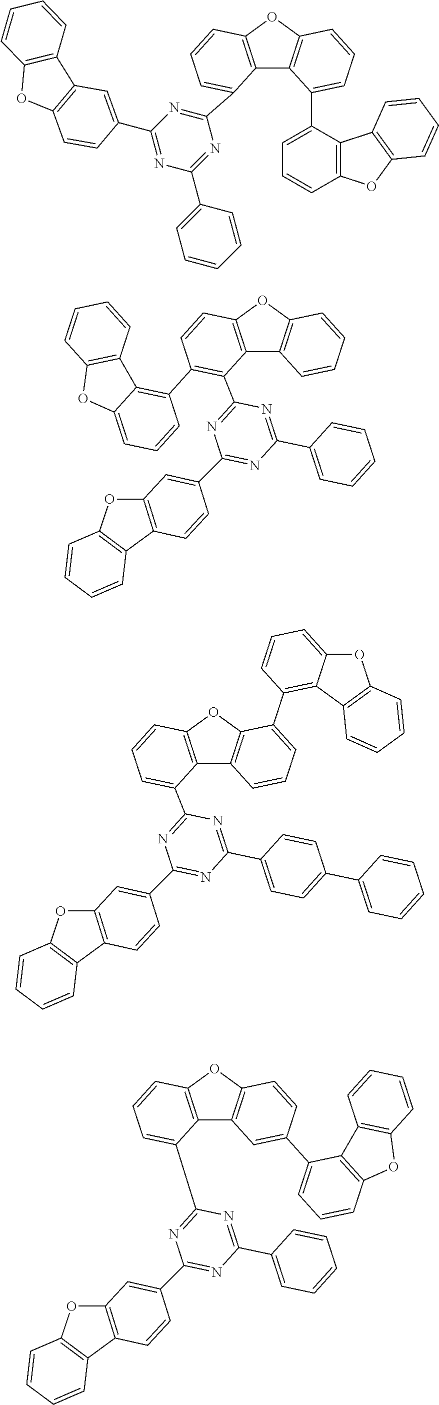

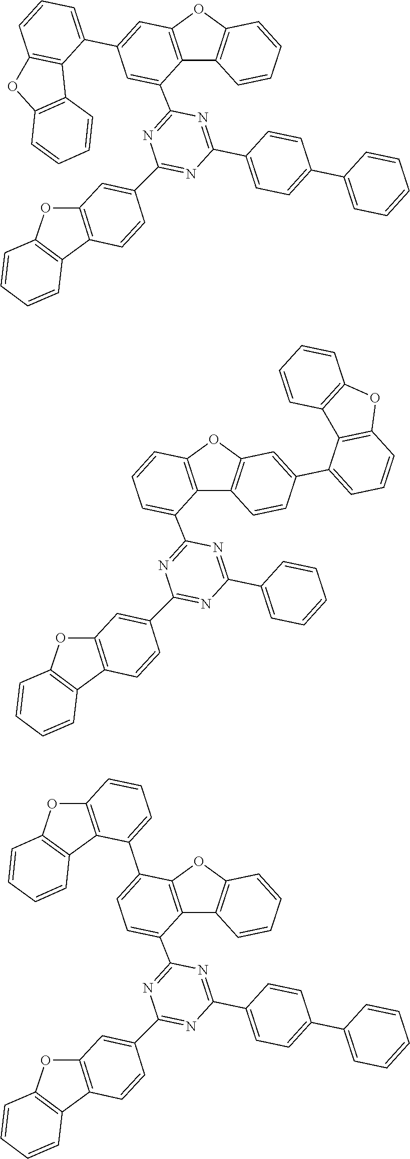 [SMILES: c1ccc(-c2ccc(-c3nc(-c4ccc5c(c4)oc4ccccc45)nc(-c4cc(-c5cccc6oc7ccccc7c56)cc5oc6ccccc6c45)n3)cc2)cc1 ; c1ccc(-c2nc(-c3ccc4c(c3)oc3ccccc34)nc(-c3cccc4oc5cc(-c6cccc7oc8ccccc8c67)ccc5c34)n2)cc1 ; c1ccc(-c2ccc(-c3nc(-c4ccc5c(c4)oc4ccccc45)nc(-c4ccc(-c5cccc6oc7ccccc7c56)c5oc6ccccc6c45)n3)cc2)cc1]

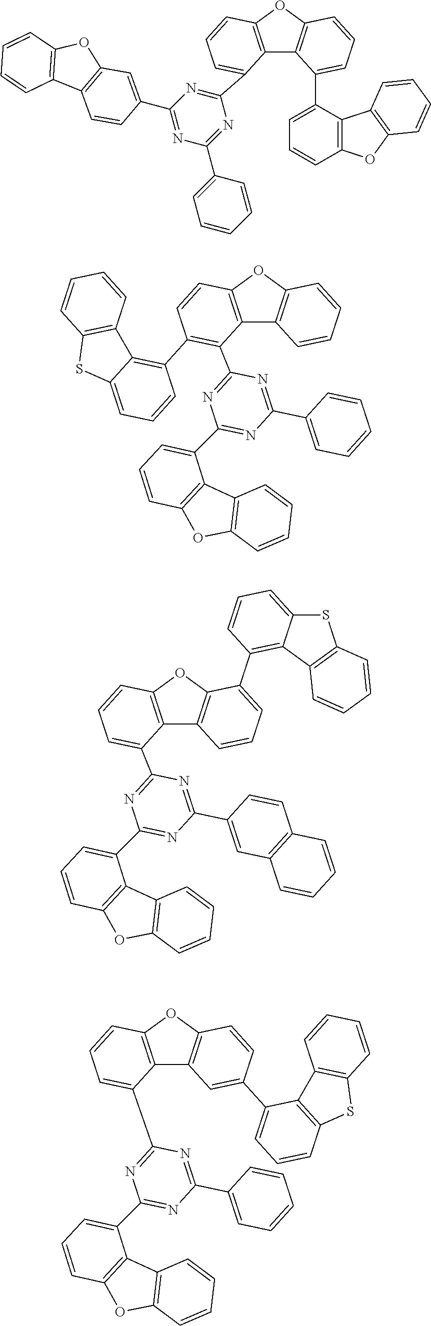

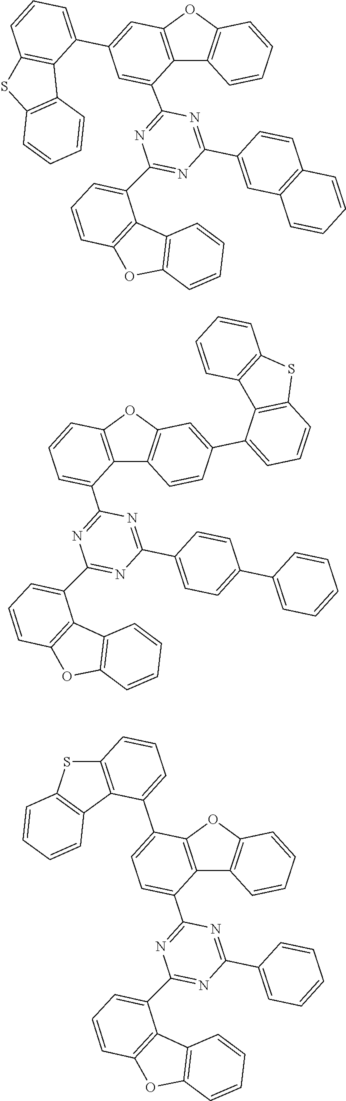

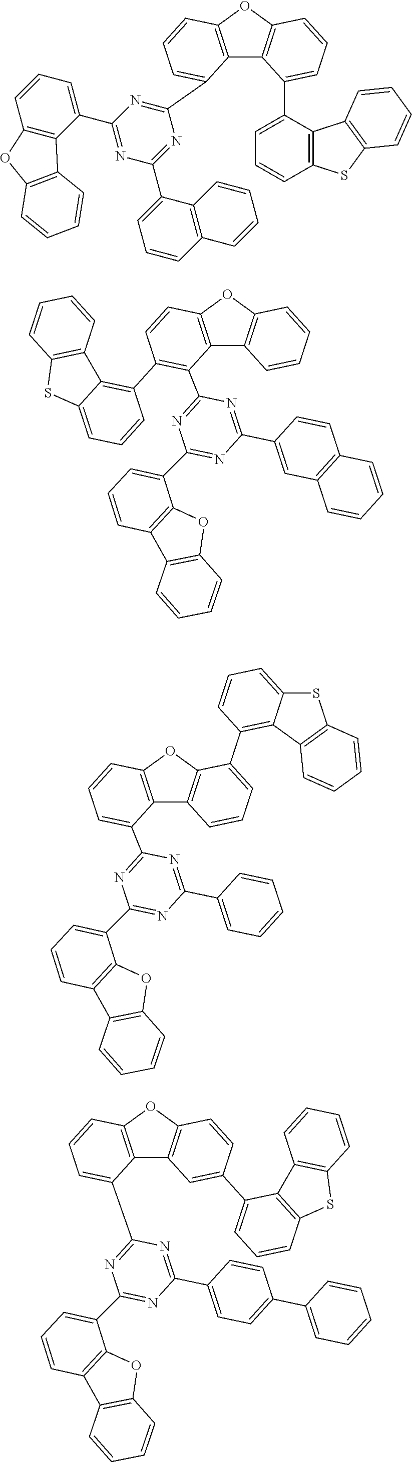

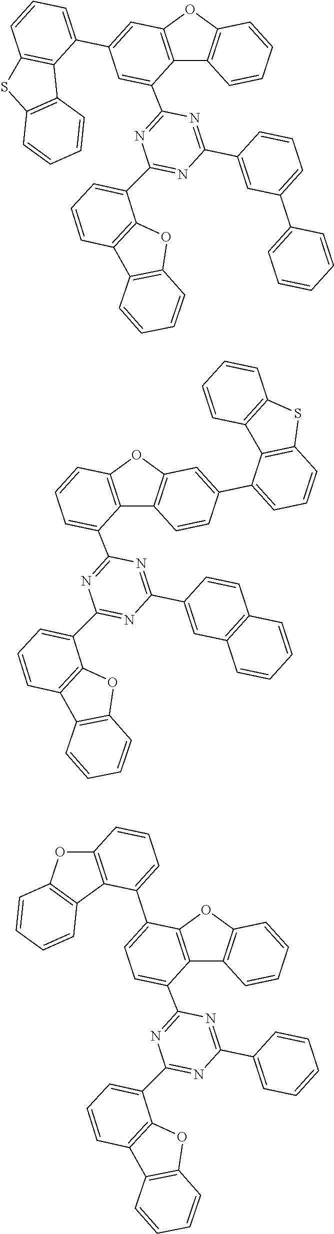 [SMILES: c1ccc(-c2cccc(-c3nc(-c4cccc5c4oc4ccccc45)nc(-c4cc(-c5cccc6sc7ccccc7c56)cc5oc6ccccc6c45)n3)c2)cc1 ; c1ccc2cc(-c3nc(-c4cccc5c4oc4ccccc45)nc(-c4cccc5oc6cc(-c7cccc8sc9ccccc9c78)ccc6c45)n3)ccc2c1 ; c1ccc(-c2nc(-c3cccc4c3oc3ccccc34)nc(-c3ccc(-c4cccc5oc6ccccc6c45)c4oc5ccccc5c34)n2)cc1]

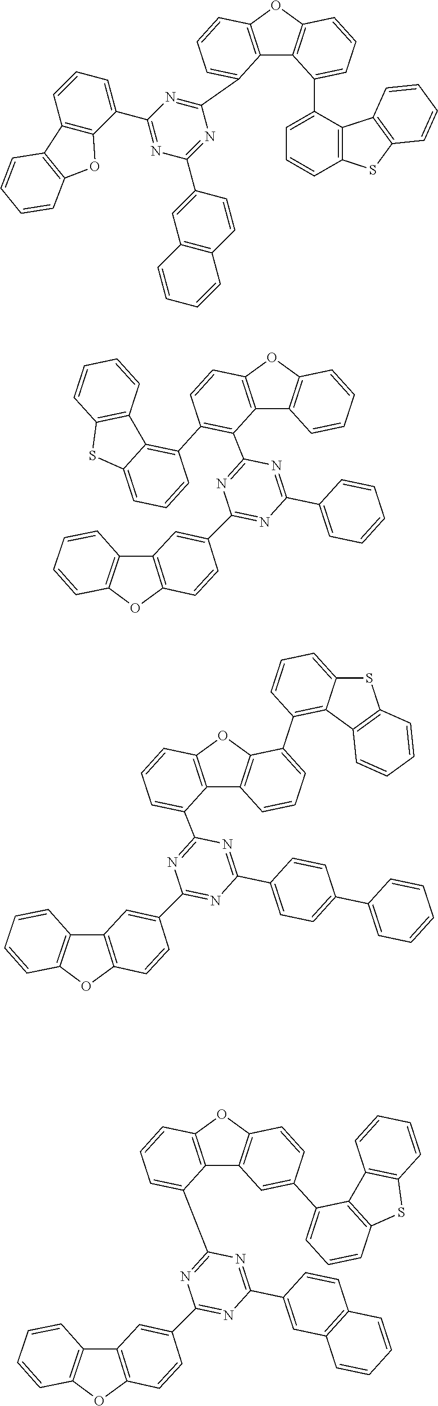 [SMILES: c1ccc2cc(-c3nc(-c4cccc5c4oc4ccccc45)nc(-c4cccc5oc6cccc(-c7cccc8sc9ccccc9c78)c6c45)n3)ccc2c1 ; c1ccc(-c2nc(-c3ccc4oc5ccccc5c4c3)nc(-c3c(-c4cccc5sc6ccccc6c45)ccc4oc5ccccc5c34)n2)cc1 ; c1ccc(-c2ccc(-c3nc(-c4ccc5oc6ccccc6c5c4)nc(-c4cccc5oc6c(-c7cccc8sc9ccccc9c78)cccc6c45)n3)cc2)cc1 ; c1ccc2cc(-c3nc(-c4ccc5oc6ccccc6c5c4)nc(-c4cccc5oc6ccc(-c7cccc8sc9ccccc9c78)cc6c45)n3)ccc2c1]

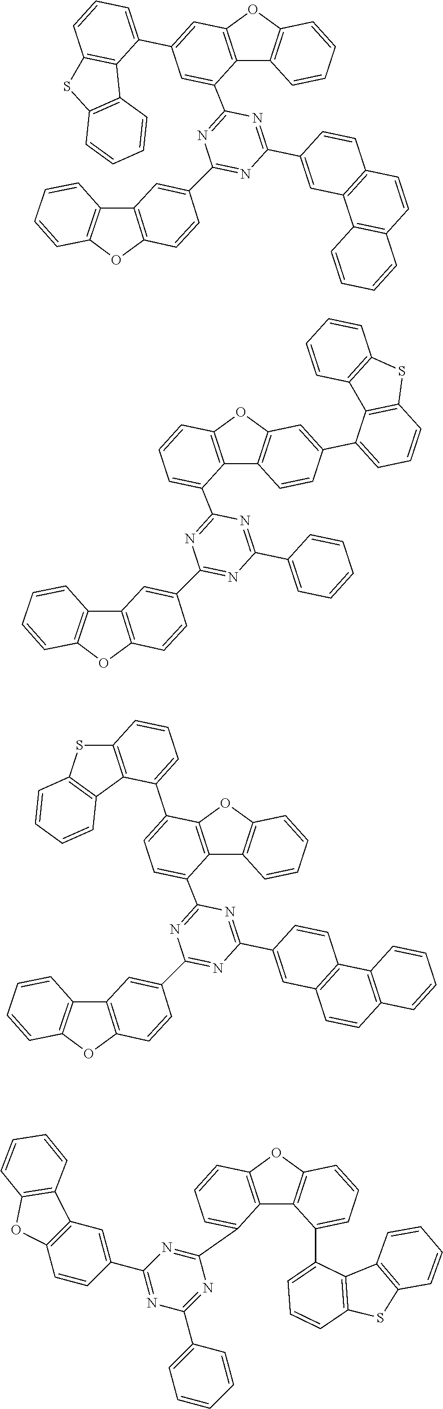

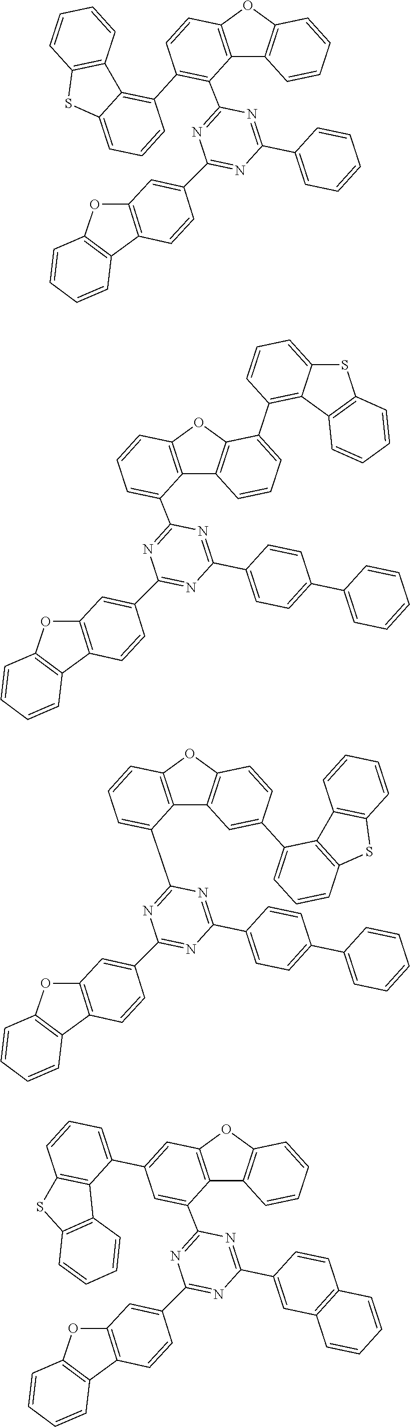 [SMILES: c1ccc(-c2nc(-c3ccc4c(c3)oc3ccccc34)nc(-c3c(-c4cccc5sc6ccccc6c45)ccc4oc5ccccc5c34)n2)cc1 ; c1ccc(-c2ccc(-c3nc(-c4ccc5c(c4)oc4ccccc45)nc(-c4cccc5oc6c(-c7cccc8sc9ccccc9c78)cccc6c45)n3)cc2)cc1 ; c1ccc(-c2ccc(-c3nc(-c4ccc5c(c4)oc4ccccc45)nc(-c4cccc5oc6ccc(-c7cccc8sc9ccccc9c78)cc6c45)n3)cc2)cc1 ; c1ccc2cc(-c3nc(-c4ccc5c(c4)oc4ccccc45)nc(-c4cc(-c5cccc6sc7ccccc7c56)cc5oc6ccccc6c45)n3)ccc2c1]

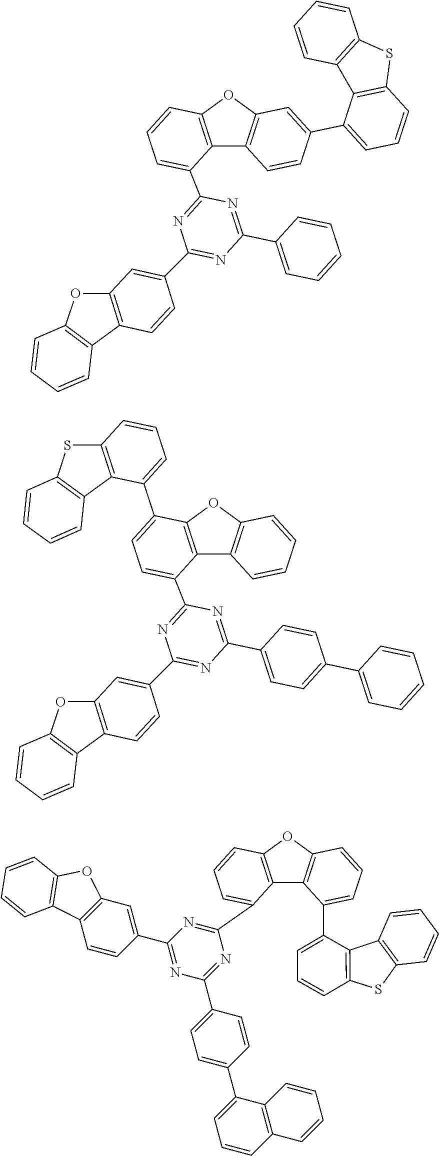 [SMILES: c1ccc(-c2nc(-c3ccc4c(c3)oc3ccccc34)nc(-c3cccc4oc5cc(-c6cccc7sc8ccccc8c67)ccc5c34)n2)cc1 ; c1ccc(-c2ccc(-c3nc(-c4ccc5c(c4)oc4ccccc45)nc(-c4ccc(-c5cccc6sc7ccccc7c56)c5oc6ccccc6c45)n3)cc2)cc1 ; c1ccc2c(-c3ccc(-c4nc(-c5ccc6c(c5)oc5ccccc56)nc(-c5cccc6oc7cccc(-c8cccc9sc%10ccccc%10c89)c7c56)n4)cc3)cccc2c1]

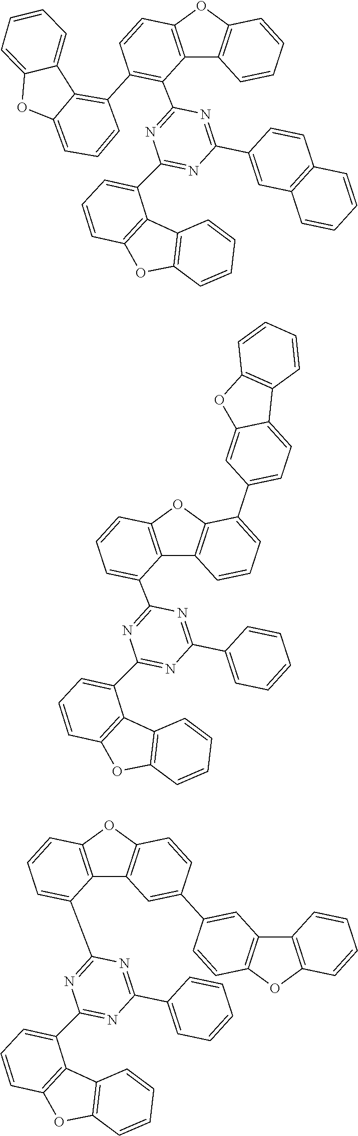 [SMILES: c1ccc2cc(-c3nc(-c4cccc5oc6ccccc6c45)nc(-c4c(-c5cccc6oc7ccccc7c56)ccc5oc6ccccc6c45)n3)ccc2c1 ; c1ccc(-c2nc(-c3cccc4oc5ccccc5c34)nc(-c3cccc4oc5c(-c6ccc7c(c6)oc6ccccc67)cccc5c34)n2)cc1 ; c1ccc(-c2nc(-c3cccc4oc5ccccc5c34)nc(-c3cccc4oc5ccc(-c6ccc7oc8ccccc8c7c6)cc5c34)n2)cc1]

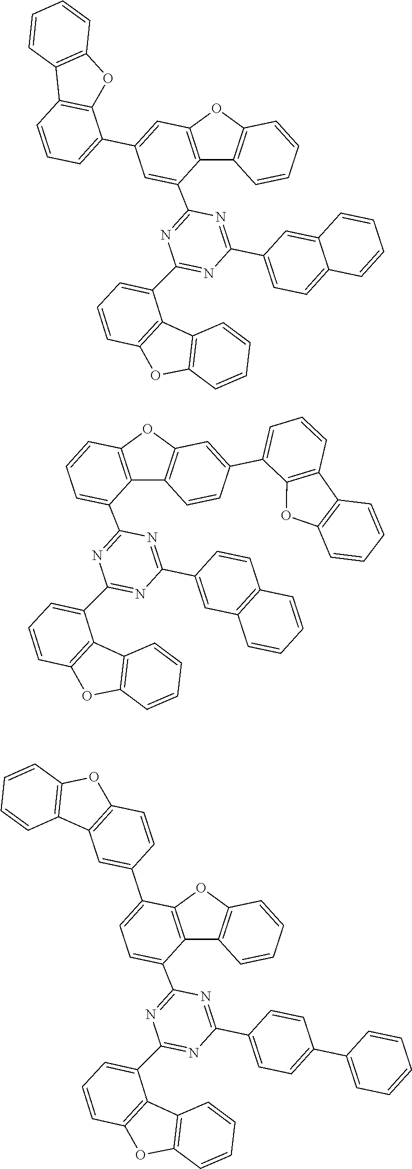 [SMILES: c1ccc2cc(-c3nc(-c4cccc5oc6ccccc6c45)nc(-c4cc(-c5cccc6c5oc5ccccc56)cc5oc6ccccc6c45)n3)ccc2c1 ; c1ccc2cc(-c3nc(-c4cccc5oc6ccccc6c45)nc(-c4cccc5oc6cc(-c7cccc8c7oc7ccccc78)ccc6c45)n3)ccc2c1 ; c1ccc(-c2ccc(-c3nc(-c4cccc5oc6ccccc6c45)nc(-c4ccc(-c5ccc6oc7ccccc7c6c5)c5oc6ccccc6c45)n3)cc2)cc1]

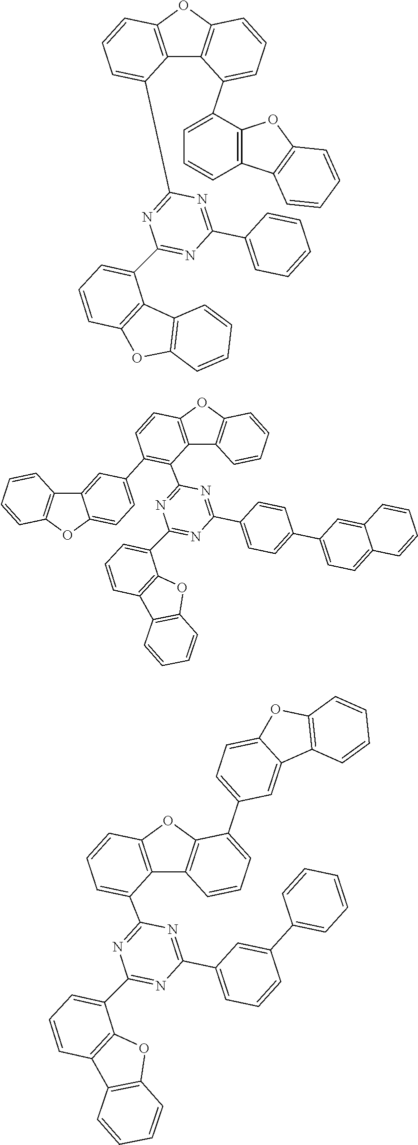

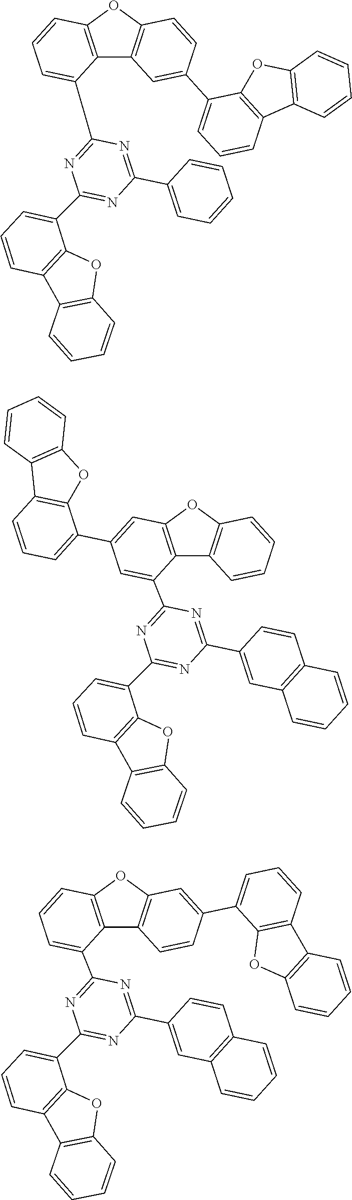 [SMILES: c1ccc(-c2nc(-c3cccc4c3oc3ccccc34)nc(-c3cccc4oc5ccc(-c6cccc7c6oc6ccccc67)cc5c34)n2)cc1 ; c1ccc2cc(-c3nc(-c4cccc5c4oc4ccccc45)nc(-c4cc(-c5cccc6c5oc5ccccc56)cc5oc6ccccc6c45)n3)ccc2c1 ; c1ccc2cc(-c3nc(-c4cccc5c4oc4ccccc45)nc(-c4cccc5oc6cc(-c7cccc8c7oc7ccccc78)ccc6c45)n3)ccc2c1]

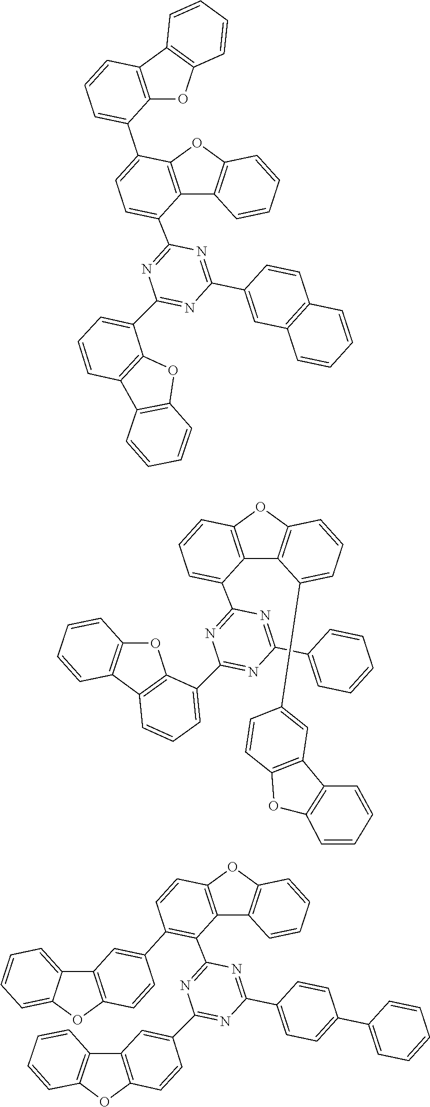 [SMILES: c1ccc2cc(-c3nc(-c4cccc5c4oc4ccccc45)nc(-c4ccc(-c5cccc6c5oc5ccccc56)c5oc6ccccc6c45)n3)ccc2c1 ; c1ccc(-c2nc(-c3cccc4c3oc3ccccc34)nc(-c3cccc4oc5cccc(-c6ccc7oc8ccccc8c7c6)c5c34)n2)cc1 ; c1ccc(-c2ccc(-c3nc(-c4ccc5oc6ccccc6c5c4)nc(-c4c(-c5ccc6oc7ccccc7c6c5)ccc5oc6ccccc6c45)n3)cc2)cc1]

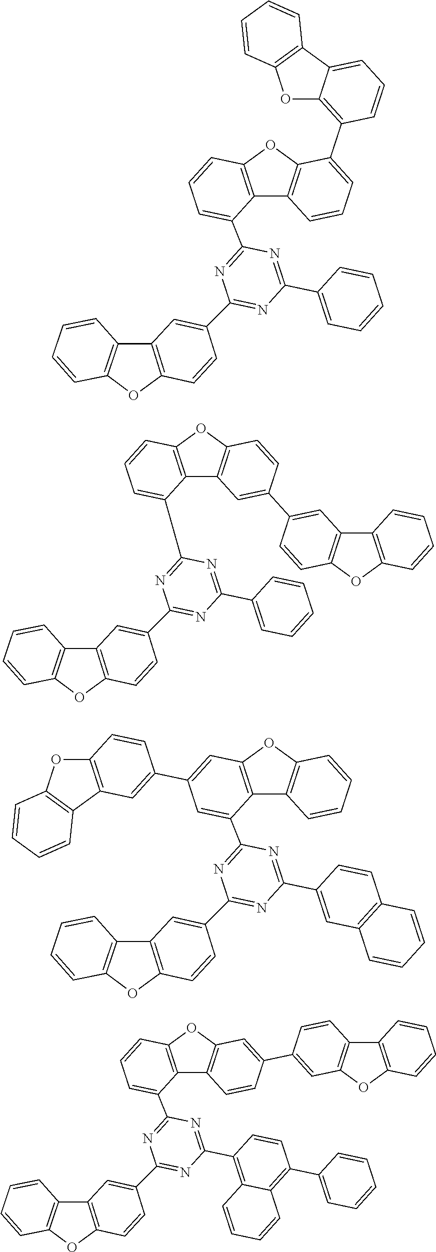 [SMILES: c1ccc(-c2nc(-c3ccc4oc5ccccc5c4c3)nc(-c3cccc4oc5c(-c6cccc7c6oc6ccccc67)cccc5c34)n2)cc1 ; c1ccc(-c2nc(-c3ccc4oc5ccccc5c4c3)nc(-c3cccc4oc5ccc(-c6ccc7oc8ccccc8c7c6)cc5c34)n2)cc1 ; c1ccc2cc(-c3nc(-c4ccc5oc6ccccc6c5c4)nc(-c4cc(-c5ccc6oc7ccccc7c6c5)cc5oc6ccccc6c45)n3)ccc2c1 ; c1ccc(-c2ccc(-c3nc(-c4ccc5oc6ccccc6c5c4)nc(-c4cccc5oc6cc(-c7ccc8c(c7)oc7ccccc78)ccc6c45)n3)c3ccccc23)cc1]

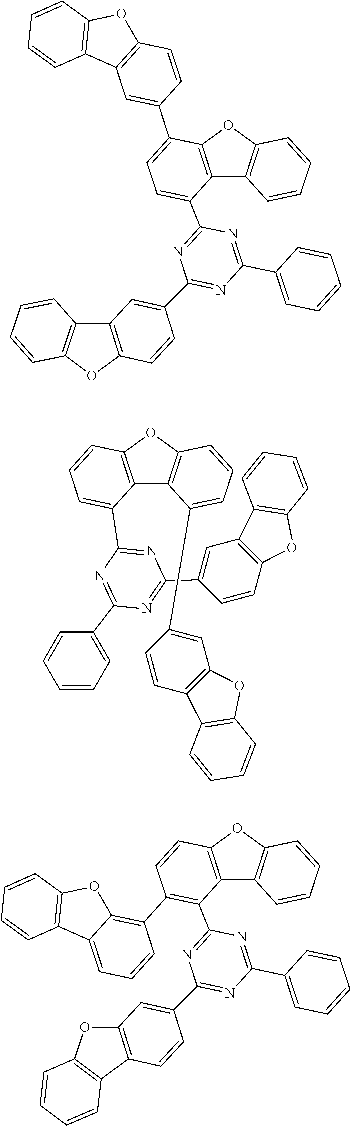 [SMILES: c1ccc(-c2nc(-c3ccc4oc5ccccc5c4c3)nc(-c3ccc(-c4ccc5oc6ccccc6c5c4)c4oc5ccccc5c34)n2)cc1 ; c1ccc(-c2nc(-c3ccc4oc5ccccc5c4c3)nc(-c3cccc4oc5cccc(-c6ccc7c(c6)oc6ccccc67)c5c34)n2)cc1 ; c1ccc(-c2nc(-c3ccc4c(c3)oc3ccccc34)nc(-c3c(-c4cccc5c4oc4ccccc45)ccc4oc5ccccc5c34)n2)cc1]

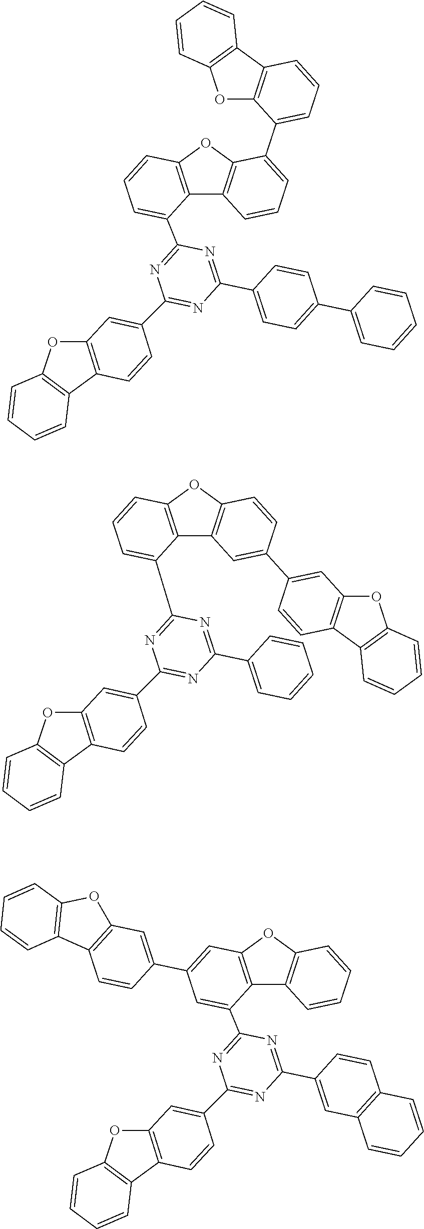 [SMILES: c1ccc(-c2ccc(-c3nc(-c4ccc5c(c4)oc4ccccc45)nc(-c4cccc5oc6c(-c7cccc8c7oc7ccccc78)cccc6c45)n3)cc2)cc1 ; c1ccc(-c2nc(-c3ccc4c(c3)oc3ccccc34)nc(-c3cccc4oc5ccc(-c6ccc7c(c6)oc6ccccc67)cc5c34)n2)cc1 ; c1ccc2cc(-c3nc(-c4ccc5c(c4)oc4ccccc45)nc(-c4cc(-c5ccc6c(c5)oc5ccccc56)cc5oc6ccccc6c45)n3)ccc2c1]

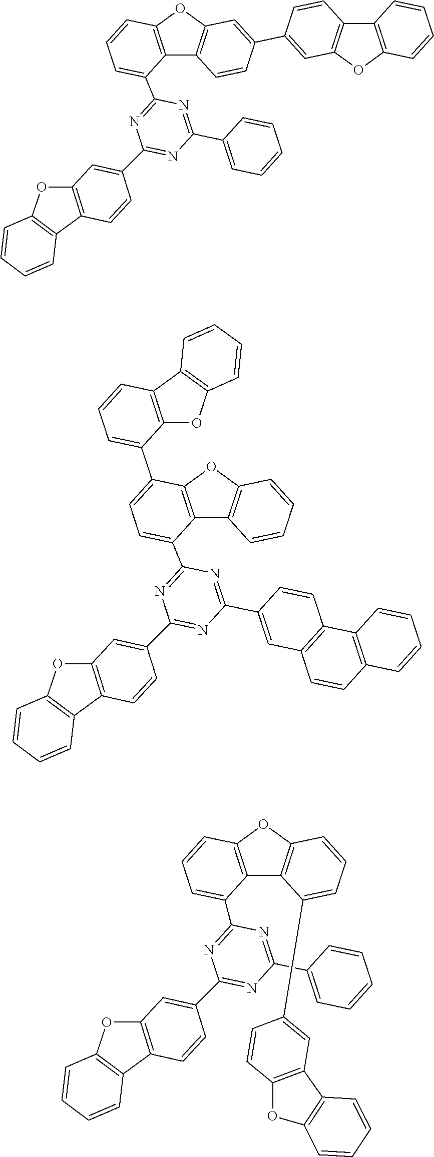 [SMILES: c1ccc(-c2nc(-c3ccc4c(c3)oc3ccccc34)nc(-c3cccc4oc5cc(-c6ccc7c(c6)oc6ccccc67)ccc5c34)n2)cc1 ; c1ccc2c(c1)ccc1cc(-c3nc(-c4ccc5c(c4)oc4ccccc45)nc(-c4ccc(-c5cccc6c5oc5ccccc56)c5oc6ccccc6c45)n3)ccc12 ; c1ccc(-c2nc(-c3ccc4c(c3)oc3ccccc34)nc(-c3cccc4oc5cccc(-c6ccc7oc8ccccc8c7c6)c5c34)n2)cc1]

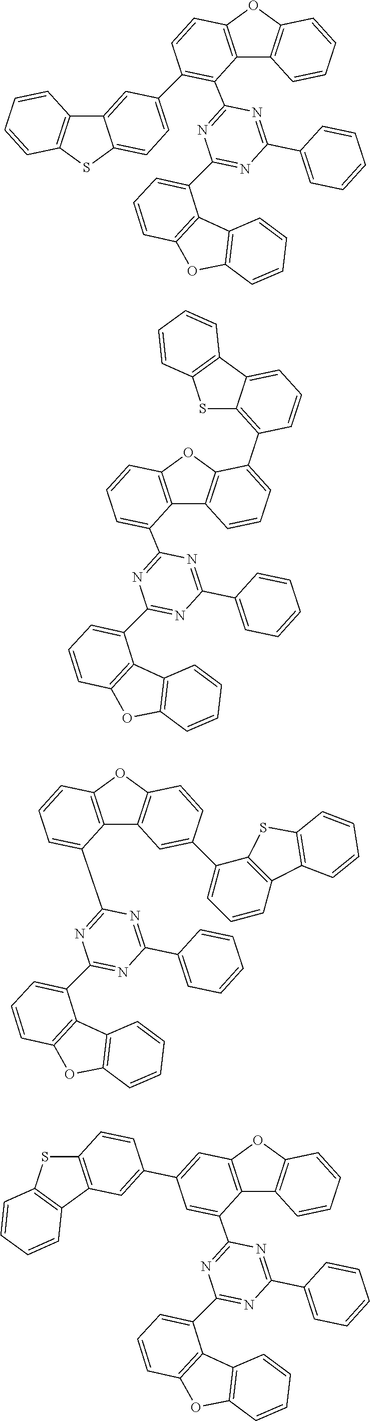

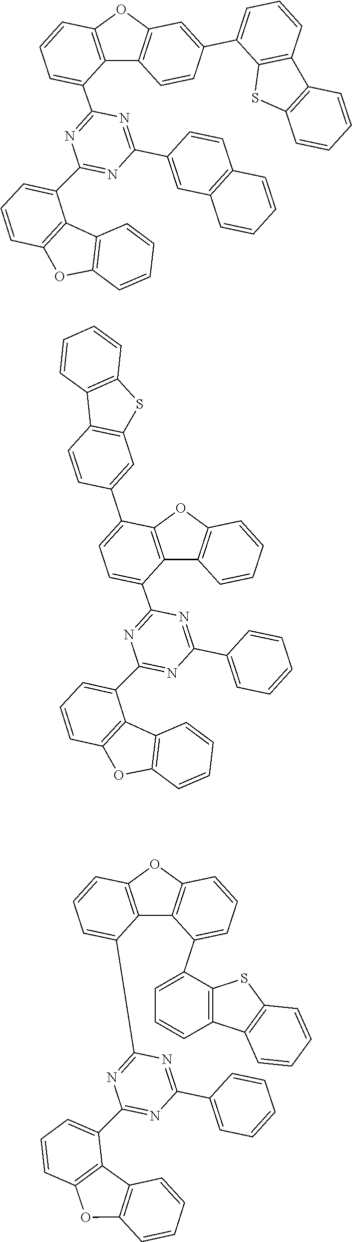

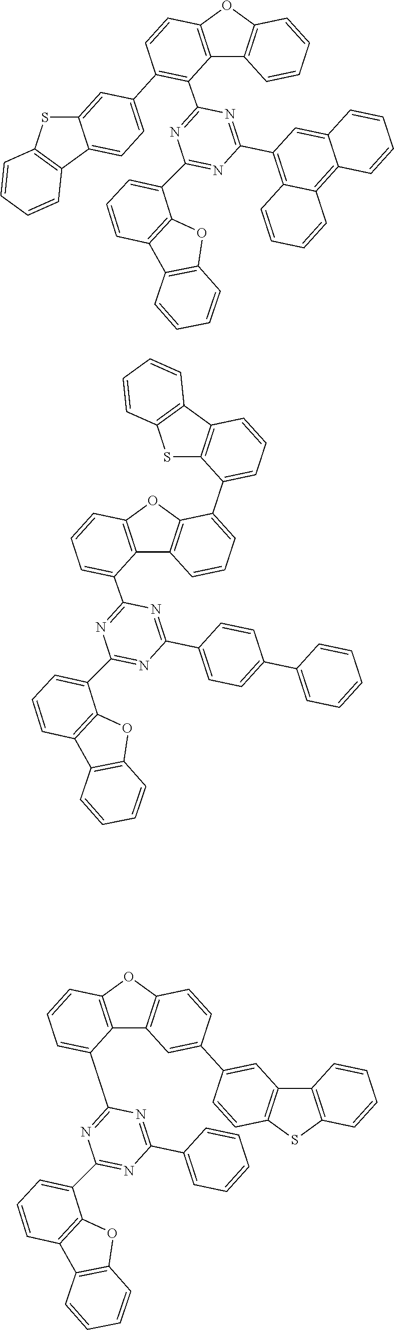

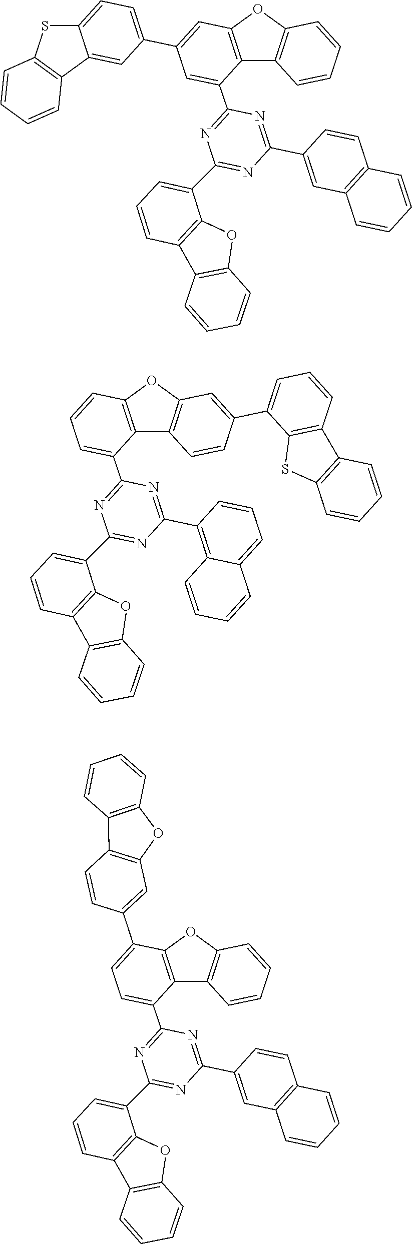 [SMILES: c1ccc2cc(-c3nc(-c4cccc5c4oc4ccccc45)nc(-c4cc(-c5ccc6sc7ccccc7c6c5)cc5oc6ccccc6c45)n3)ccc2c1 ; c1ccc2c(-c3nc(-c4cccc5c4oc4ccccc45)nc(-c4cccc5oc6cc(-c7cccc8c7sc7ccccc78)ccc6c45)n3)cccc2c1 ; c1ccc2cc(-c3nc(-c4cccc5c4oc4ccccc45)nc(-c4ccc(-c5ccc6c(c5)oc5ccccc56)c5oc6ccccc6c45)n3)ccc2c1]

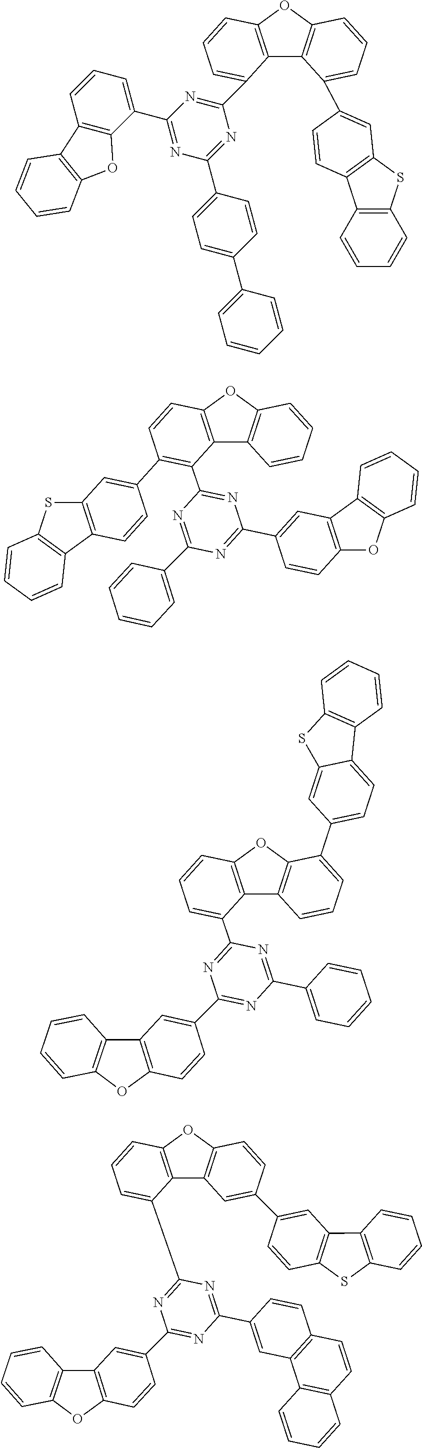

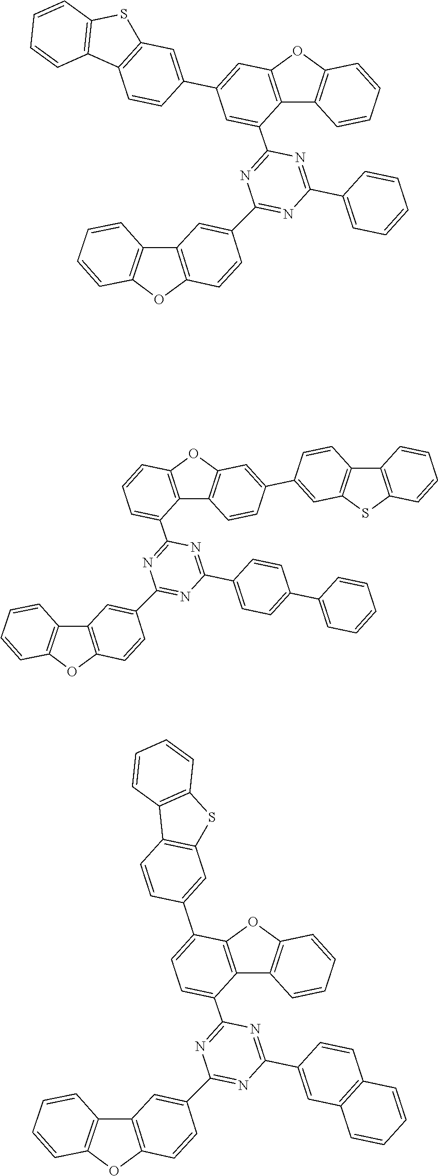 [SMILES: c1ccc(-c2nc(-c3ccc4oc5ccccc5c4c3)nc(-c3cc(-c4ccc5c(c4)sc4ccccc45)cc4oc5ccccc5c34)n2)cc1 ; c1ccc(-c2ccc(-c3nc(-c4ccc5oc6ccccc6c5c4)nc(-c4cccc5oc6cc(-c7ccc8c(c7)sc7ccccc78)ccc6c45)n3)cc2)cc1 ; c1ccc2cc(-c3nc(-c4ccc5oc6ccccc6c5c4)nc(-c4ccc(-c5ccc6c(c5)sc5ccccc56)c5oc6ccccc6c45)n3)ccc2c1]

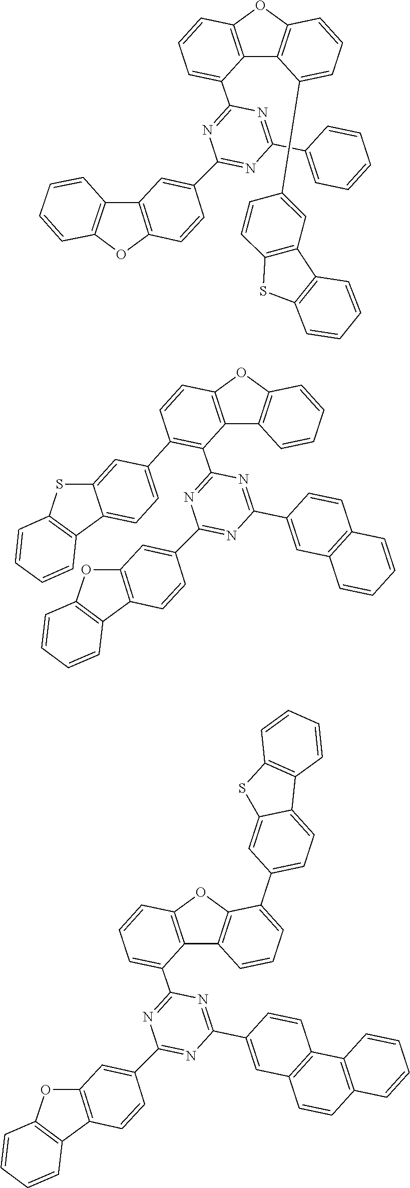 [SMILES: c1ccc(-c2nc(-c3ccc4oc5ccccc5c4c3)nc(-c3cccc4oc5cccc(-c6ccc7sc8ccccc8c7c6)c5c34)n2)cc1 ; c1ccc2cc(-c3nc(-c4ccc5c(c4)oc4ccccc45)nc(-c4c(-c5ccc6c(c5)sc5ccccc56)ccc5oc6ccccc6c45)n3)ccc2c1 ; c1ccc2c(c1)ccc1cc(-c3nc(-c4ccc5c(c4)oc4ccccc45)nc(-c4cccc5oc6c(-c7ccc8c(c7)sc7ccccc78)cccc6c45)n3)ccc12]

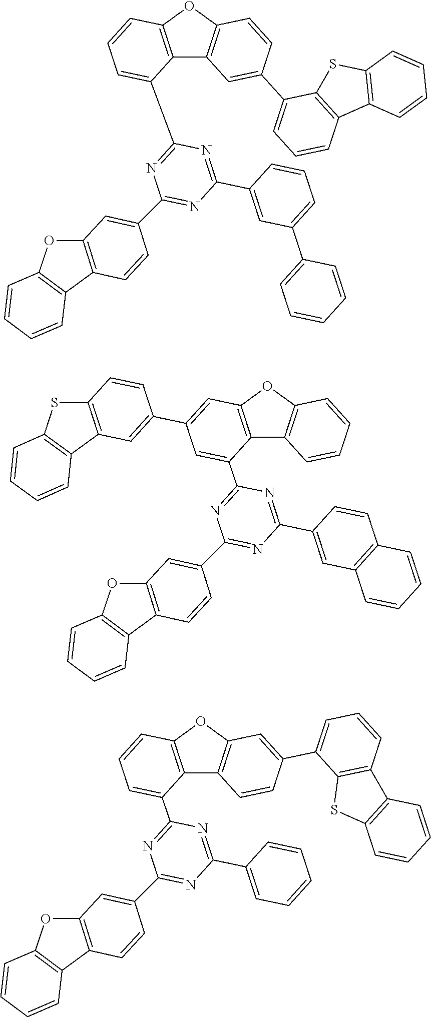 [SMILES: c1ccc(-c2cccc(-c3nc(-c4ccc5c(c4)oc4ccccc45)nc(-c4cccc5oc6ccc(-c7cccc8c7sc7ccccc78)cc6c45)n3)c2)cc1 ; c1ccc2cc(-c3nc(-c4ccc5c(c4)oc4ccccc45)nc(-c4cc(-c5ccc6sc7ccccc7c6c5)cc5oc6ccccc6c45)n3)ccc2c1 ; c1ccc(-c2nc(-c3ccc4c(c3)oc3ccccc34)nc(-c3cccc4oc5cc(-c6cccc7c6sc6ccccc67)ccc5c34)n2)cc1]

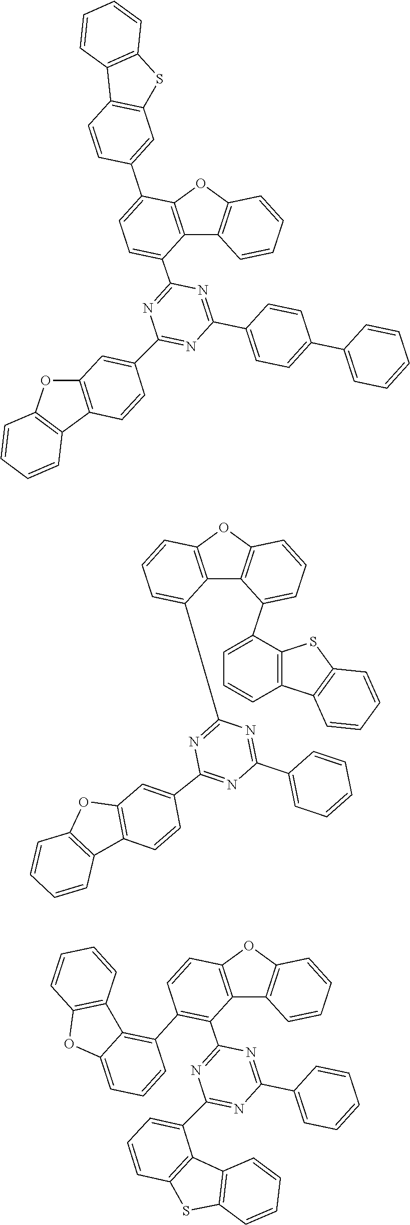 [SMILES: c1ccc(-c2ccc(-c3nc(-c4ccc5c(c4)oc4ccccc45)nc(-c4ccc(-c5ccc6c(c5)sc5ccccc56)c5oc6ccccc6c45)n3)cc2)cc1 ; c1ccc(-c2nc(-c3ccc4c(c3)oc3ccccc34)nc(-c3cccc4oc5cccc(-c6cccc7c6sc6ccccc67)c5c34)n2)cc1 ; c1ccc(-c2nc(-c3c(-c4cccc5oc6ccccc6c45)ccc4oc5ccccc5c34)nc(-c3cccc4sc5ccccc5c34)n2)cc1]

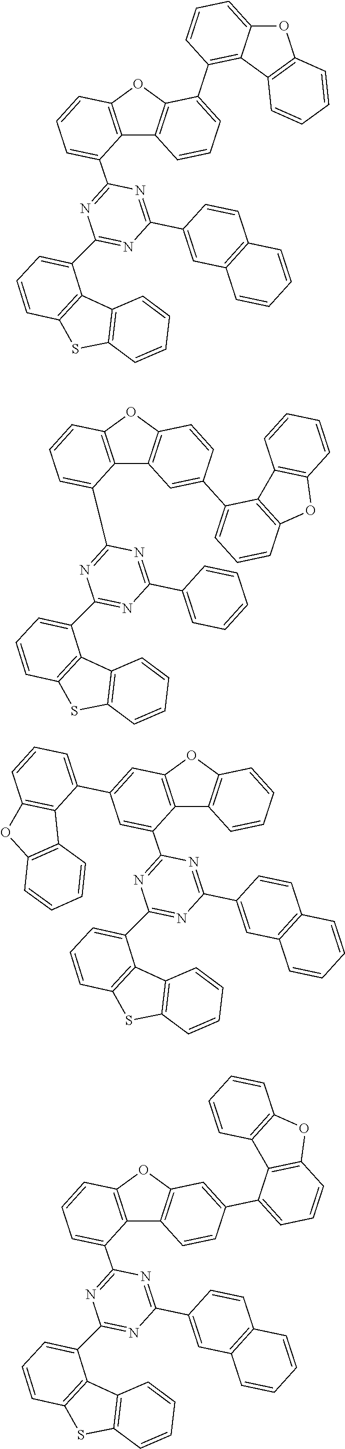

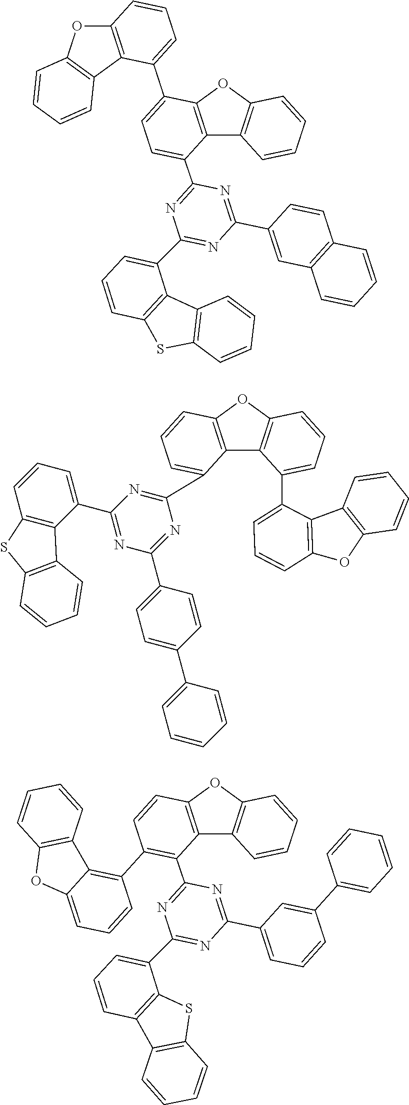

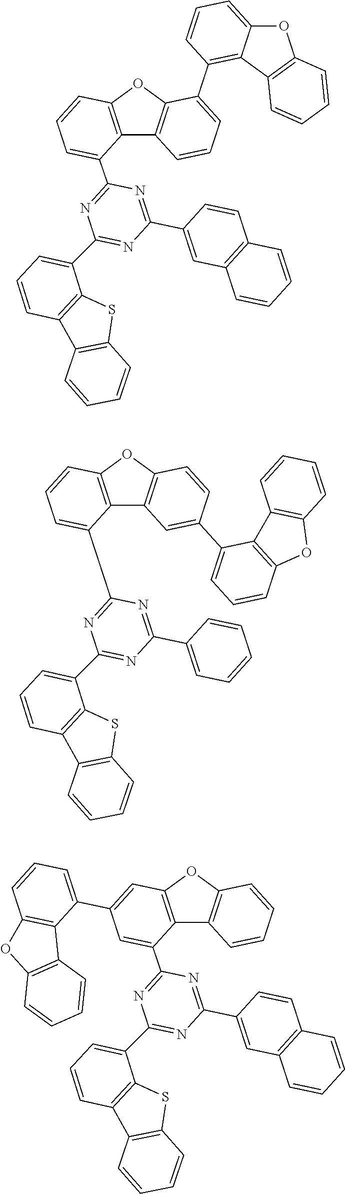 [SMILES: c1ccc2cc(-c3nc(-c4cccc5c4sc4ccccc45)nc(-c4cccc5oc6c(-c7cccc8oc9ccccc9c78)cccc6c45)n3)ccc2c1 ; c1ccc(-c2nc(-c3cccc4c3sc3ccccc34)nc(-c3cccc4oc5ccc(-c6cccc7oc8ccccc8c67)cc5c34)n2)cc1 ; c1ccc2cc(-c3nc(-c4cccc5c4sc4ccccc45)nc(-c4cc(-c5cccc6oc7ccccc7c56)cc5oc6ccccc6c45)n3)ccc2c1]

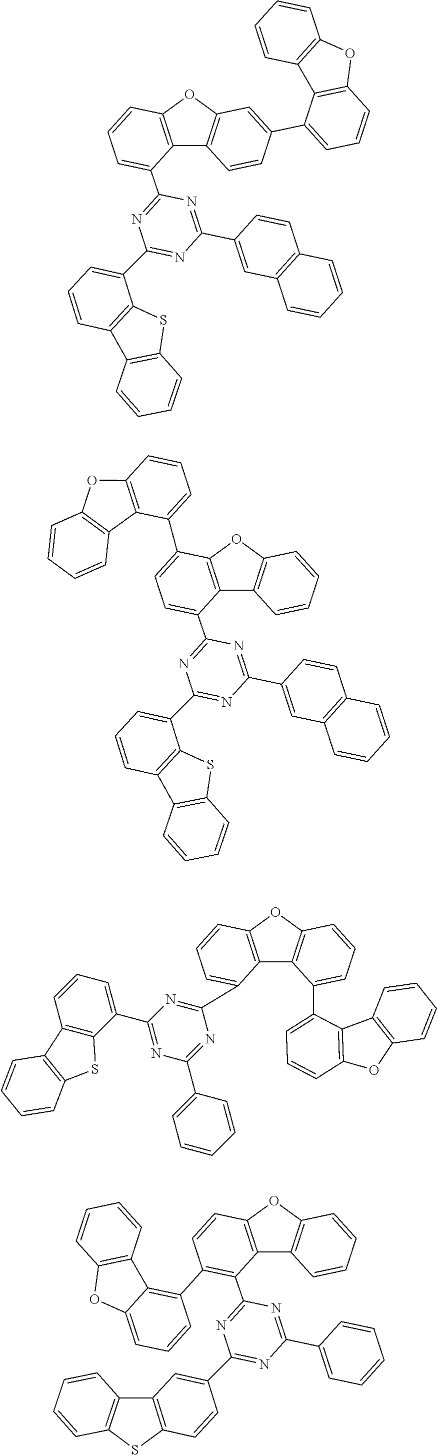

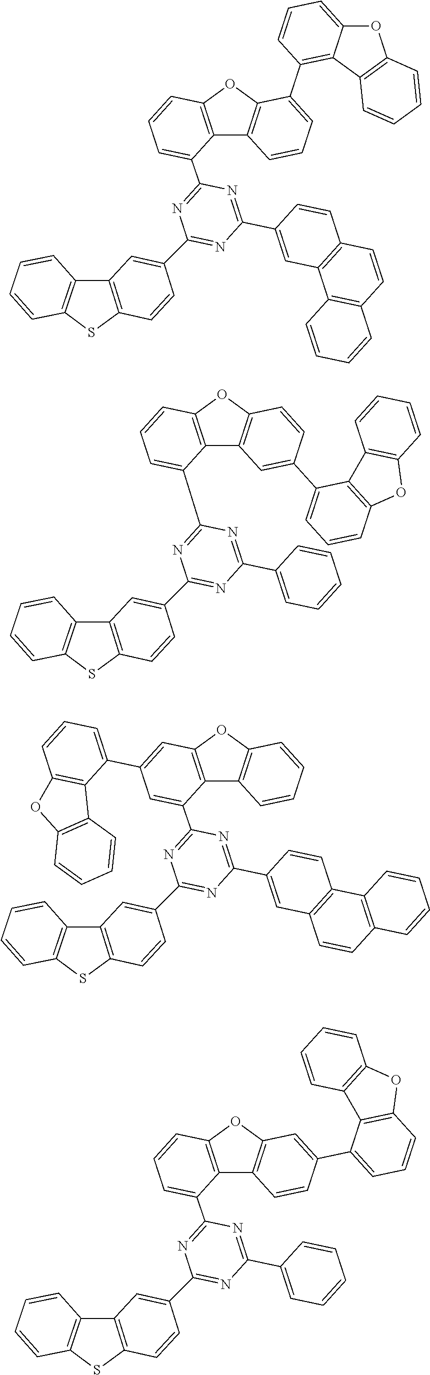

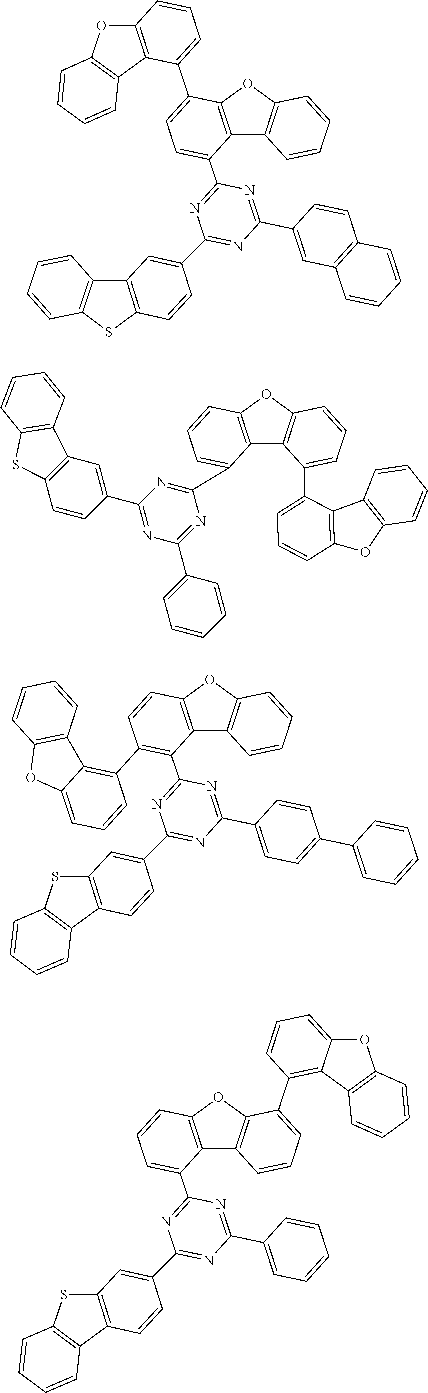

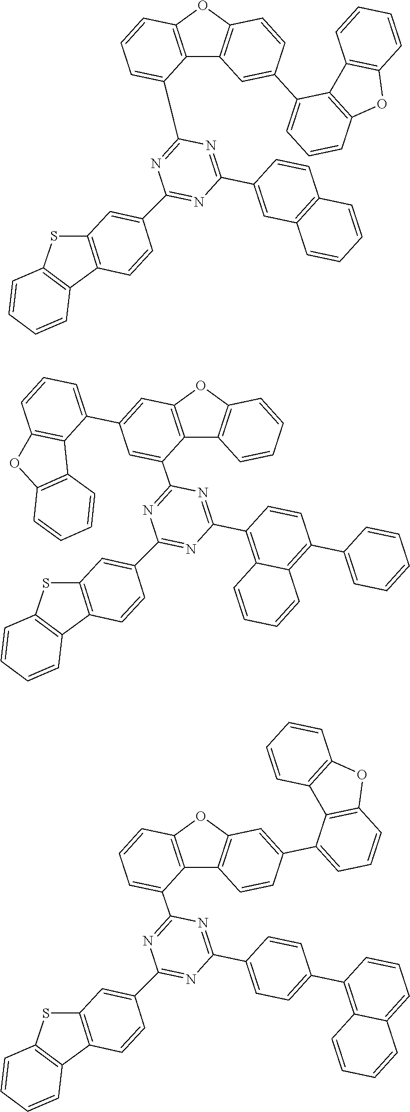

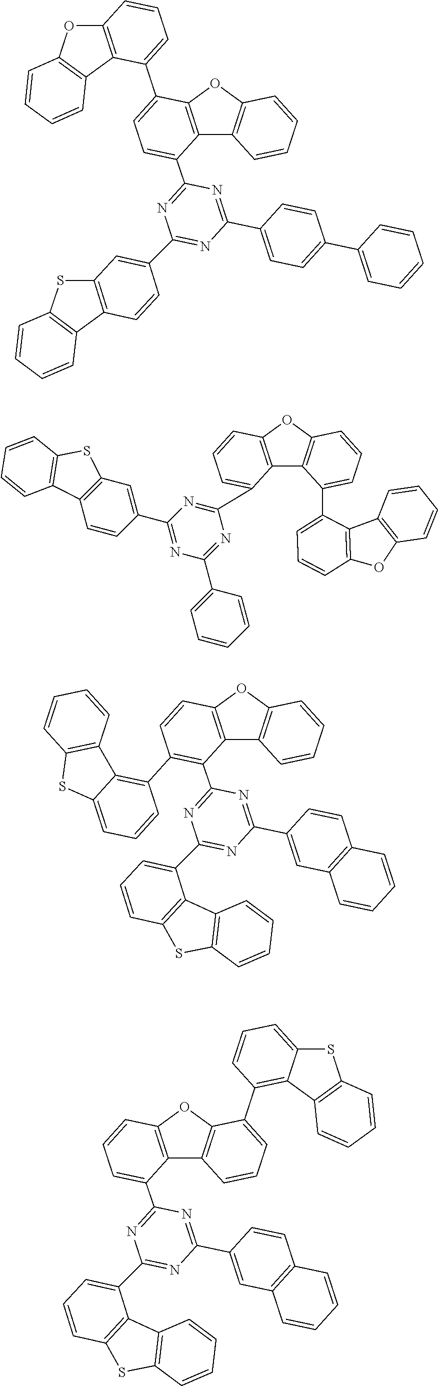

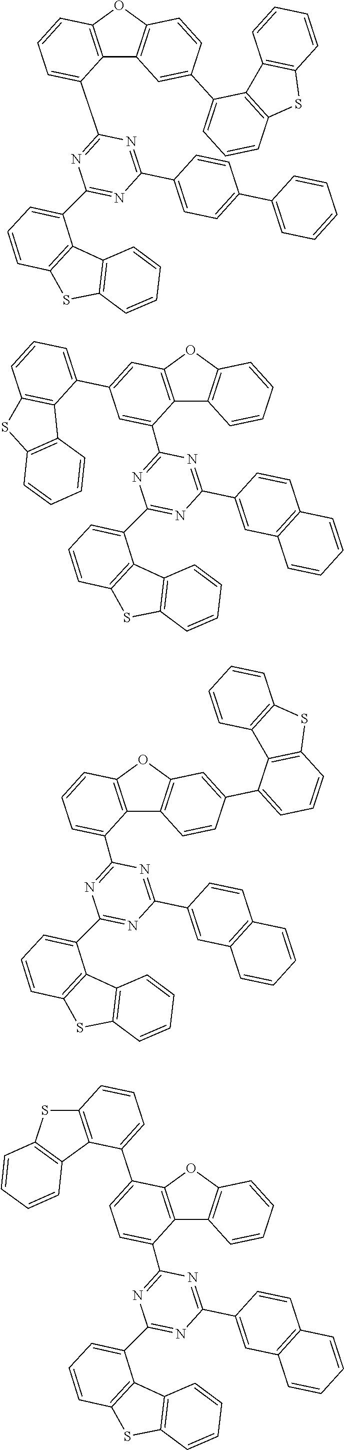 [SMILES: c1ccc(-c2ccc(-c3nc(-c4cccc5oc6ccc(-c7cccc8sc9ccccc9c78)cc6c45)nc(-c4cccc5sc6ccccc6c45)n3)cc2)cc1 ; c1ccc2cc(-c3nc(-c4cc(-c5cccc6sc7ccccc7c56)cc5oc6ccccc6c45)nc(-c4cccc5sc6ccccc6c45)n3)ccc2c1 ; c1ccc2cc(-c3nc(-c4cccc5oc6cc(-c7cccc8sc9ccccc9c78)ccc6c45)nc(-c4cccc5sc6ccccc6c45)n3)ccc2c1 ; c1ccc2cc(-c3nc(-c4cccc5sc6ccccc6c45)nc(-c4ccc(-c5cccc6sc7ccccc7c56)c5oc6ccccc6c45)n3)ccc2c1]

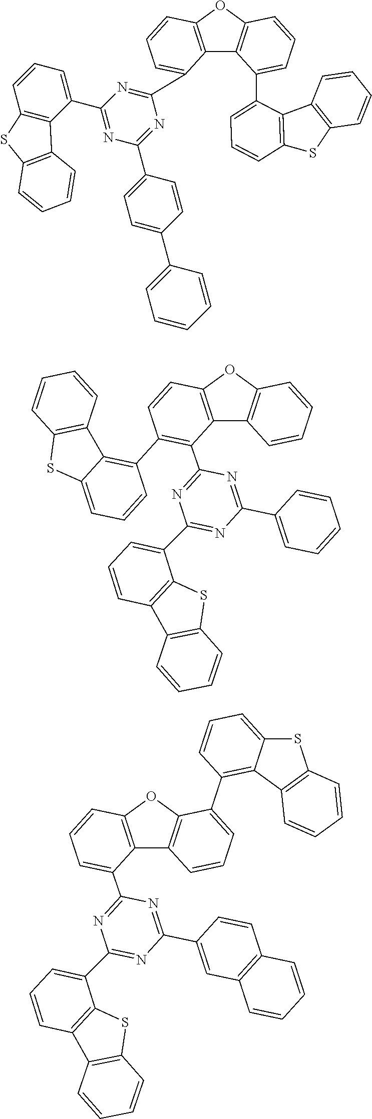

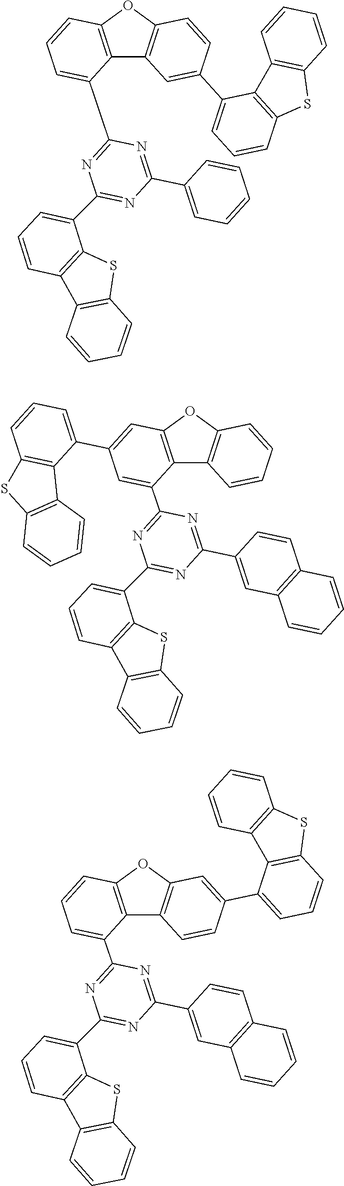 [SMILES: c1ccc(-c2nc(-c3cccc4c3sc3ccccc34)nc(-c3cccc4oc5ccc(-c6cccc7sc8ccccc8c67)cc5c34)n2)cc1 ; c1ccc2cc(-c3nc(-c4cccc5c4sc4ccccc45)nc(-c4cc(-c5cccc6sc7ccccc7c56)cc5oc6ccccc6c45)n3)ccc2c1 ; c1ccc2cc(-c3nc(-c4cccc5c4sc4ccccc45)nc(-c4cccc5oc6cc(-c7cccc8sc9ccccc9c78)ccc6c45)n3)ccc2c1]

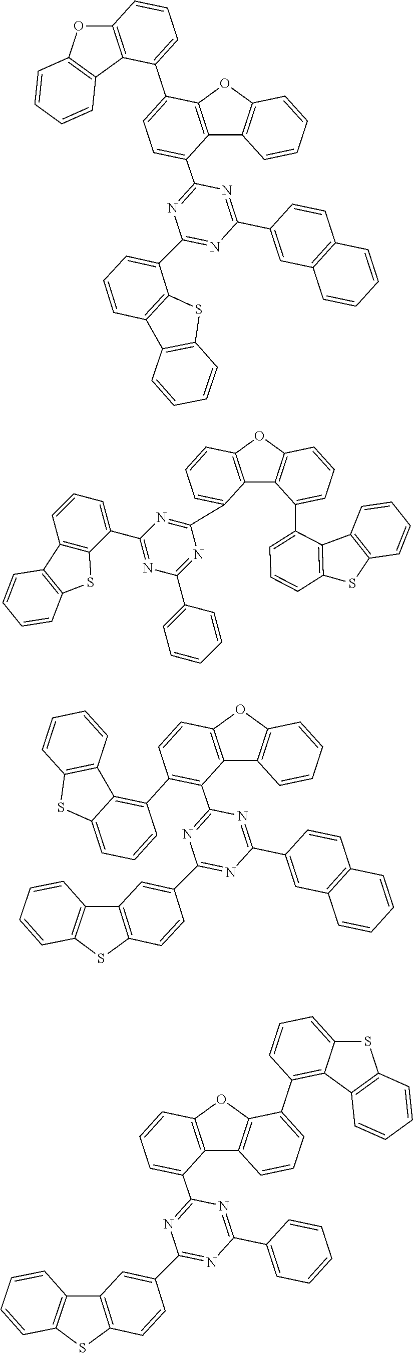 [SMILES: c1ccc2cc(-c3nc(-c4cccc5c4sc4ccccc45)nc(-c4ccc(-c5cccc6oc7ccccc7c56)c5oc6ccccc6c45)n3)ccc2c1 ; c1ccc(-c2nc(-c3cccc4c3sc3ccccc34)nc(-c3cccc4oc5cccc(-c6cccc7sc8ccccc8c67)c5c34)n2)cc1 ; c1ccc2cc(-c3nc(-c4ccc5sc6ccccc6c5c4)nc(-c4c(-c5cccc6sc7ccccc7c56)ccc5oc6ccccc6c45)n3)ccc2c1 ; c1ccc(-c2nc(-c3ccc4sc5ccccc5c4c3)nc(-c3cccc4oc5c(-c6cccc7sc8ccccc8c67)cccc5c34)n2)cc1]

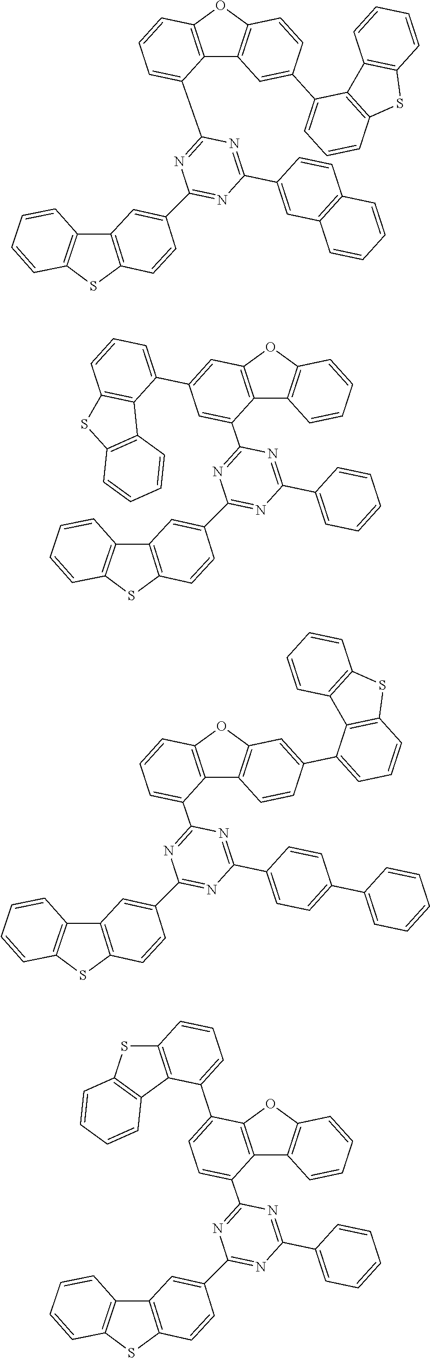 [SMILES: c1ccc2cc(-c3nc(-c4ccc5sc6ccccc6c5c4)nc(-c4cccc5oc6ccc(-c7cccc8sc9ccccc9c78)cc6c45)n3)ccc2c1 ; c1ccc(-c2nc(-c3ccc4sc5ccccc5c4c3)nc(-c3cc(-c4cccc5sc6ccccc6c45)cc4oc5ccccc5c34)n2)cc1 ; c1ccc(-c2ccc(-c3nc(-c4ccc5sc6ccccc6c5c4)nc(-c4cccc5oc6cc(-c7cccc8sc9ccccc9c78)ccc6c45)n3)cc2)cc1 ; c1ccc(-c2nc(-c3ccc4sc5ccccc5c4c3)nc(-c3ccc(-c4cccc5sc6ccccc6c45)c4oc5ccccc5c34)n2)cc1]

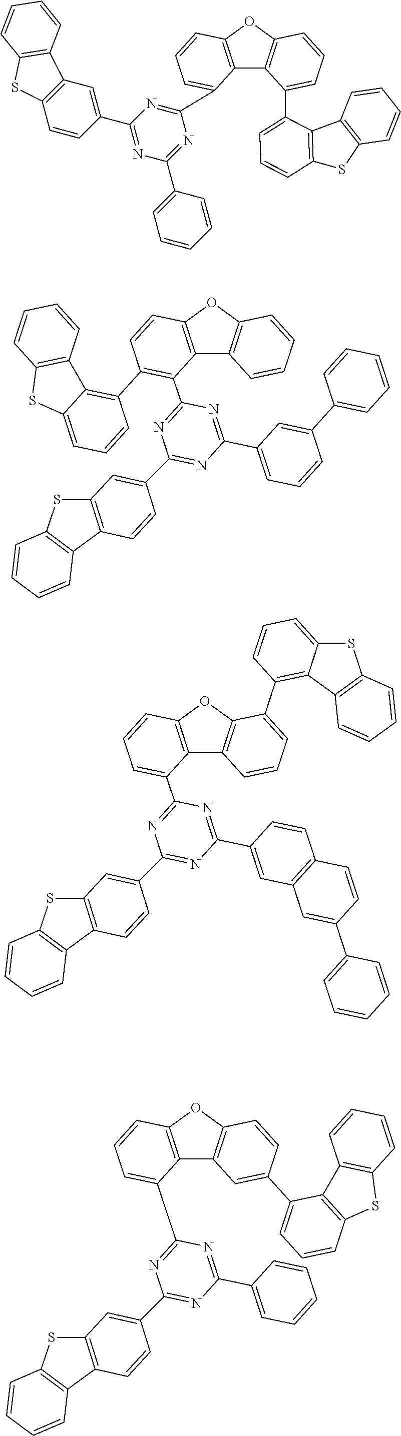 [SMILES: c1ccc(-c2nc(-c3ccc4sc5ccccc5c4c3)nc(-c3cccc4oc5cccc(-c6cccc7sc8ccccc8c67)c5c34)n2)cc1 ; c1ccc(-c2cccc(-c3nc(-c4ccc5c(c4)sc4ccccc45)nc(-c4c(-c5cccc6sc7ccccc7c56)ccc5oc6ccccc6c45)n3)c2)cc1 ; c1ccc(-c2ccc3ccc(-c4nc(-c5ccc6c(c5)sc5ccccc56)nc(-c5cccc6oc7c(-c8cccc9sc%10ccccc%10c89)cccc7c56)n4)cc3c2)cc1 ; c1ccc(-c2nc(-c3ccc4c(c3)sc3ccccc34)nc(-c3cccc4oc5ccc(-c6cccc7sc8ccccc8c67)cc5c34)n2)cc1]

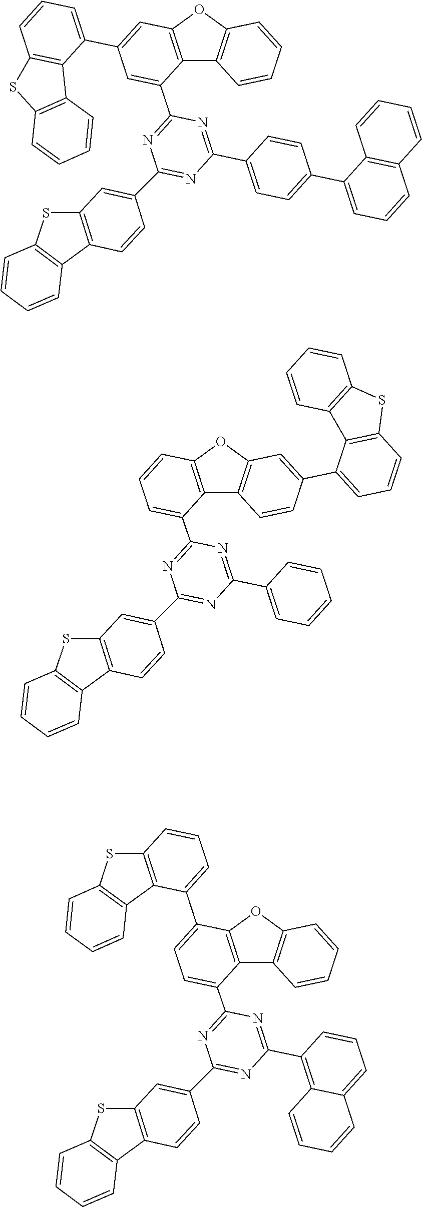 [SMILES: c1ccc2c(-c3ccc(-c4nc(-c5ccc6c(c5)sc5ccccc56)nc(-c5cc(-c6cccc7sc8ccccc8c67)cc6oc7ccccc7c56)n4)cc3)cccc2c1 ; c1ccc(-c2nc(-c3ccc4c(c3)sc3ccccc34)nc(-c3cccc4oc5cc(-c6cccc7sc8ccccc8c67)ccc5c34)n2)cc1 ; c1ccc2c(-c3nc(-c4ccc5c(c4)sc4ccccc45)nc(-c4ccc(-c5cccc6sc7ccccc7c56)c5oc6ccccc6c45)n3)cccc2c1]

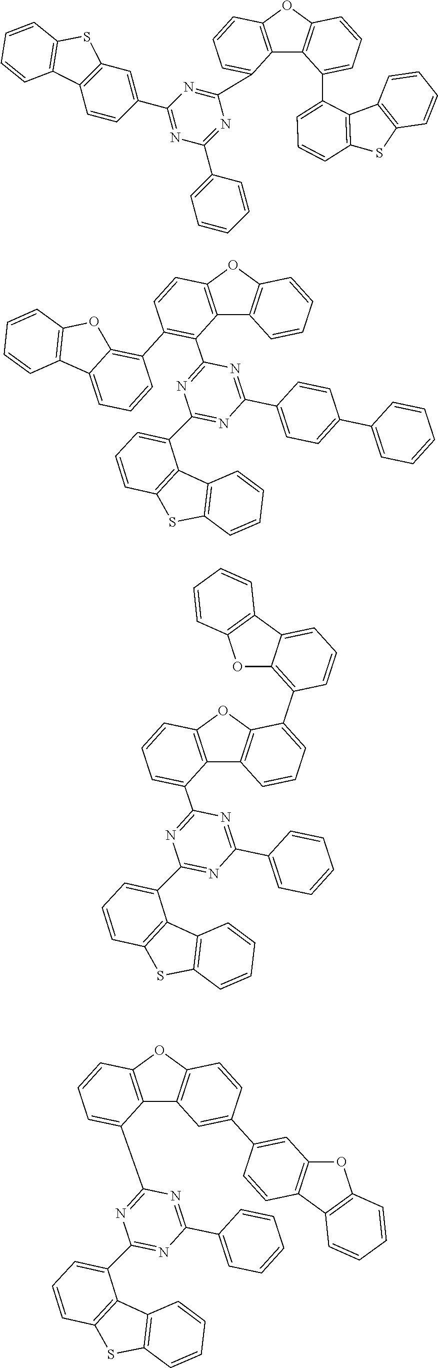

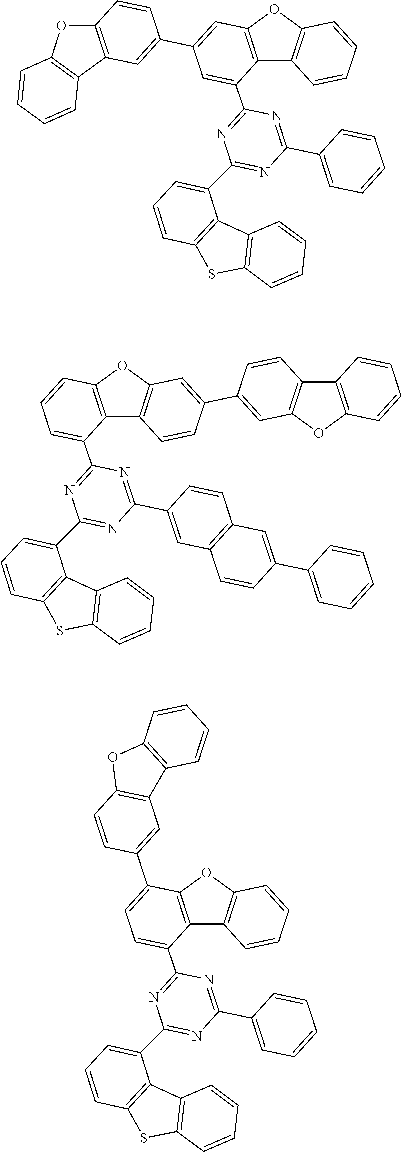 [SMILES: c1ccc(-c2nc(-c3cc(-c4ccc5oc6ccccc6c5c4)cc4oc5ccccc5c34)nc(-c3cccc4sc5ccccc5c34)n2)cc1 ; c1ccc(-c2ccc3cc(-c4nc(-c5cccc6oc7cc(-c8ccc9c(c8)oc8ccccc89)ccc7c56)nc(-c5cccc6sc7ccccc7c56)n4)ccc3c2)cc1 ; c1ccc(-c2nc(-c3cccc4sc5ccccc5c34)nc(-c3ccc(-c4ccc5oc6ccccc6c5c4)c4oc5ccccc5c34)n2)cc1]

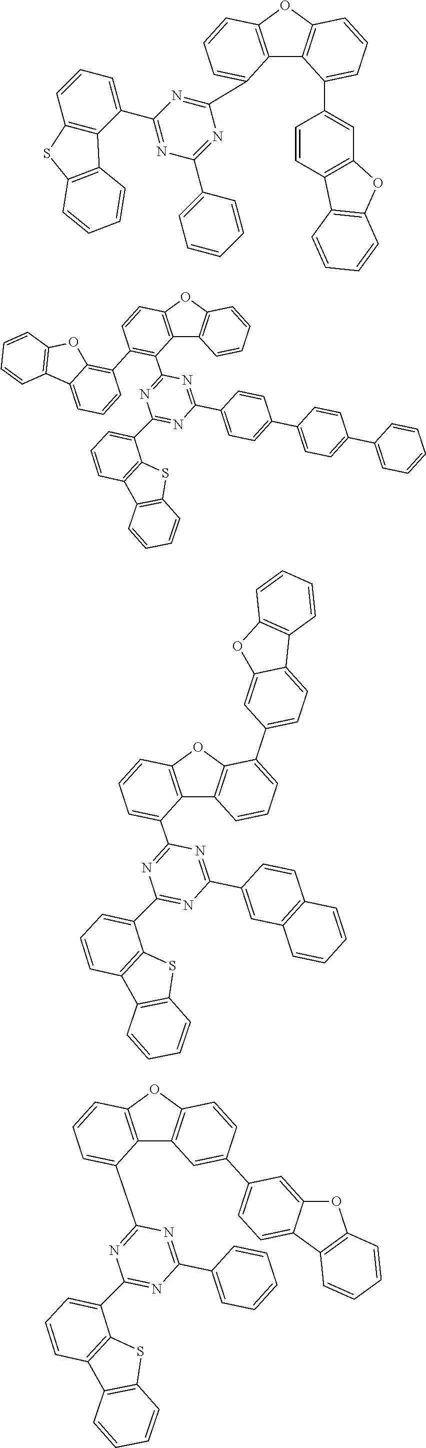

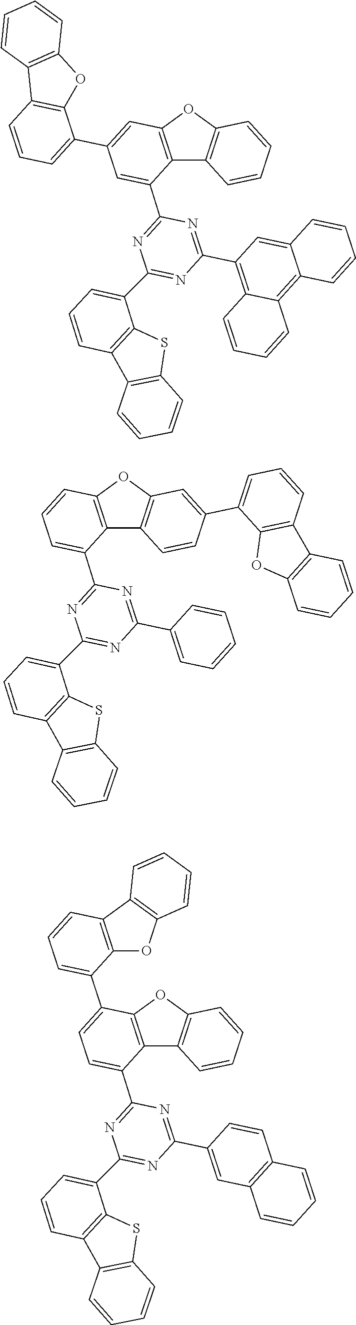

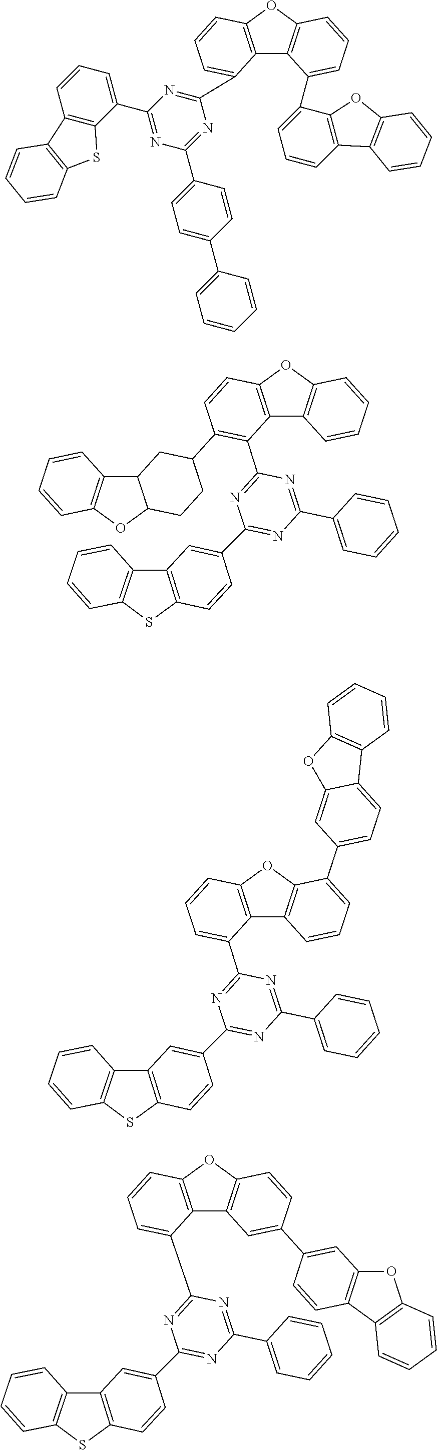 [SMILES: c1ccc(-c2ccc(-c3nc(-c4cccc5c4sc4ccccc45)nc(-c4cccc5oc6cccc(-c7cccc8c7oc7ccccc78)c6c45)n3)cc2)cc1 ; c1ccc(-c2nc(-c3ccc4sc5ccccc5c4c3)nc(-c3c(C4CCC5Oc6ccccc6C5C4)ccc4oc5ccccc5c34)n2)cc1 ; c1ccc(-c2nc(-c3ccc4sc5ccccc5c4c3)nc(-c3cccc4oc5c(-c6ccc7c(c6)oc6ccccc67)cccc5c34)n2)cc1 ; c1ccc(-c2nc(-c3ccc4sc5ccccc5c4c3)nc(-c3cccc4oc5ccc(-c6ccc7c(c6)oc6ccccc67)cc5c34)n2)cc1]

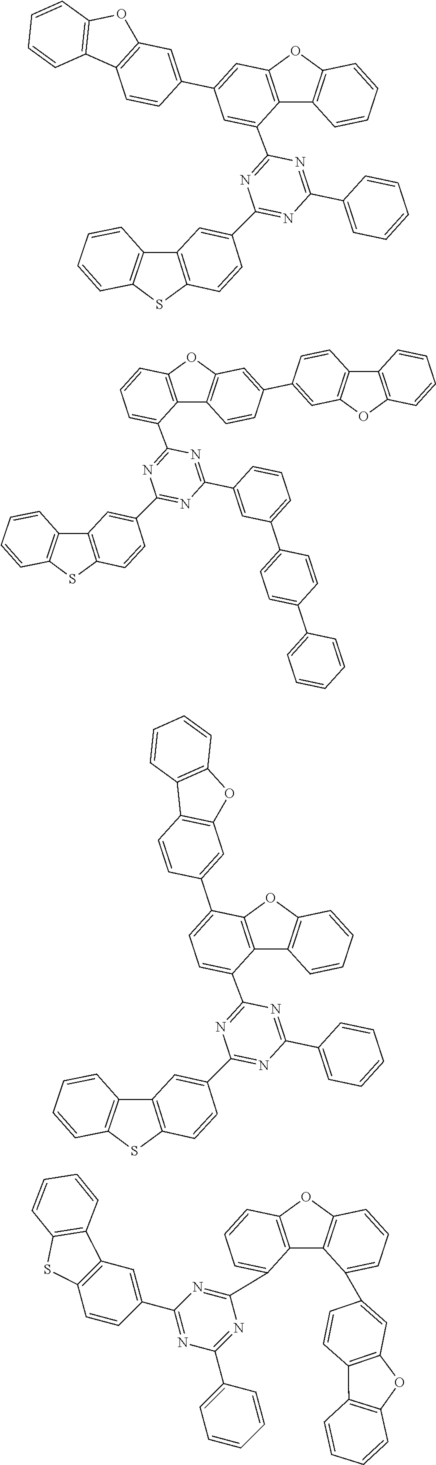

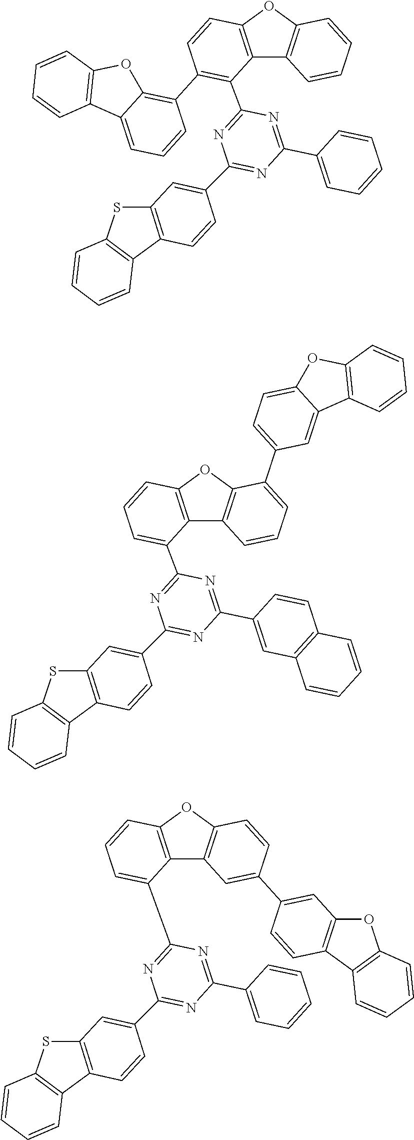 [SMILES: c1ccc(-c2nc(-c3ccc4c(c3)sc3ccccc34)nc(-c3c(-c4cccc5c4oc4ccccc45)ccc4oc5ccccc5c34)n2)cc1 ; c1ccc2cc(-c3nc(-c4ccc5c(c4)sc4ccccc45)nc(-c4cccc5oc6c(-c7ccc8oc9ccccc9c8c7)cccc6c45)n3)ccc2c1 ; c1ccc(-c2nc(-c3ccc4c(c3)sc3ccccc34)nc(-c3cccc4oc5ccc(-c6ccc7c(c6)oc6ccccc67)cc5c34)n2)cc1]

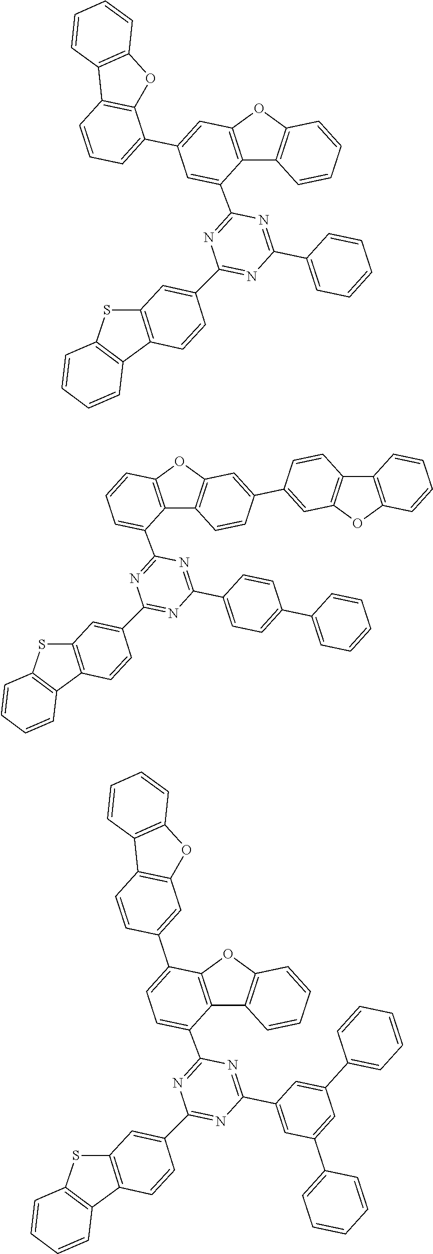 [SMILES: c1ccc(-c2nc(-c3ccc4c(c3)sc3ccccc34)nc(-c3cc(-c4cccc5c4oc4ccccc45)cc4oc5ccccc5c34)n2)cc1 ; c1ccc(-c2ccc(-c3nc(-c4ccc5c(c4)sc4ccccc45)nc(-c4cccc5oc6cc(-c7ccc8c(c7)oc7ccccc78)ccc6c45)n3)cc2)cc1 ; c1ccc(-c2cc(-c3ccccc3)cc(-c3nc(-c4ccc5c(c4)sc4ccccc45)nc(-c4ccc(-c5ccc6c(c5)oc5ccccc56)c5oc6ccccc6c45)n3)c2)cc1]

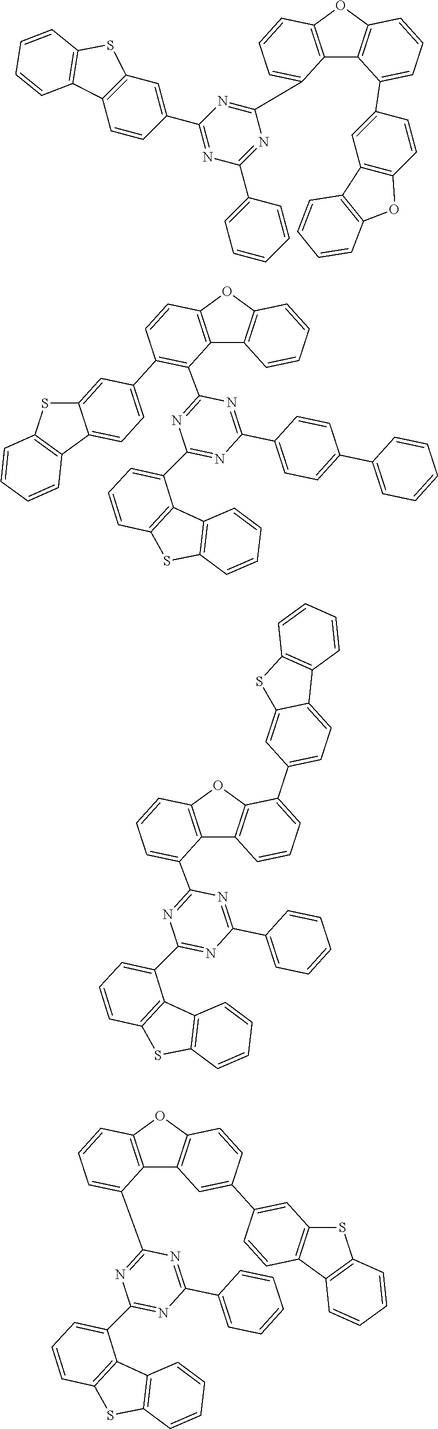

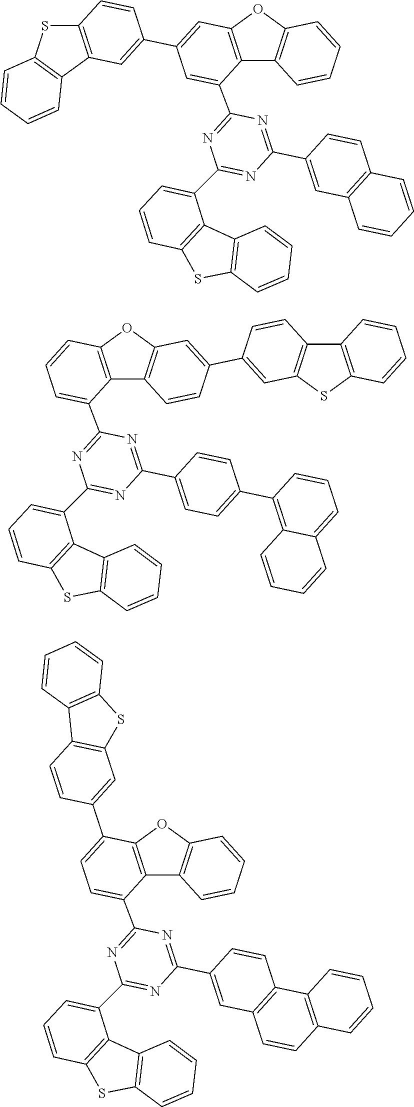

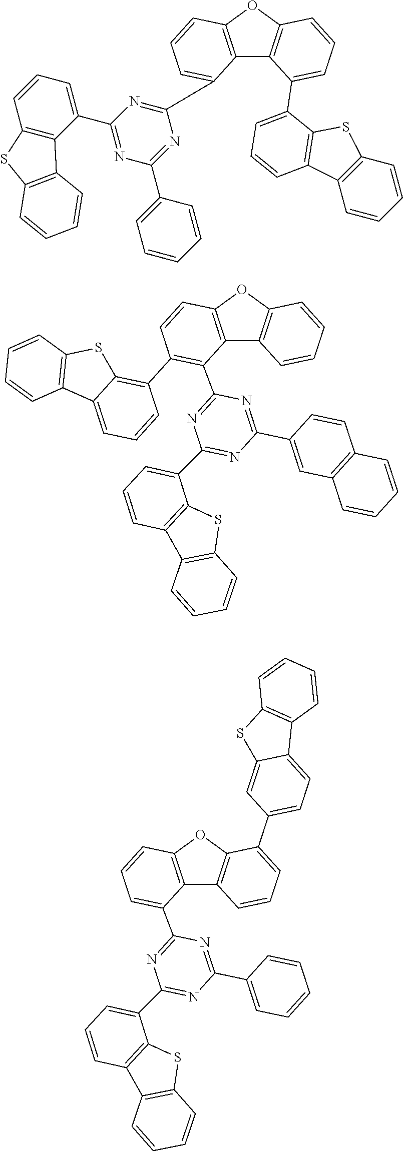

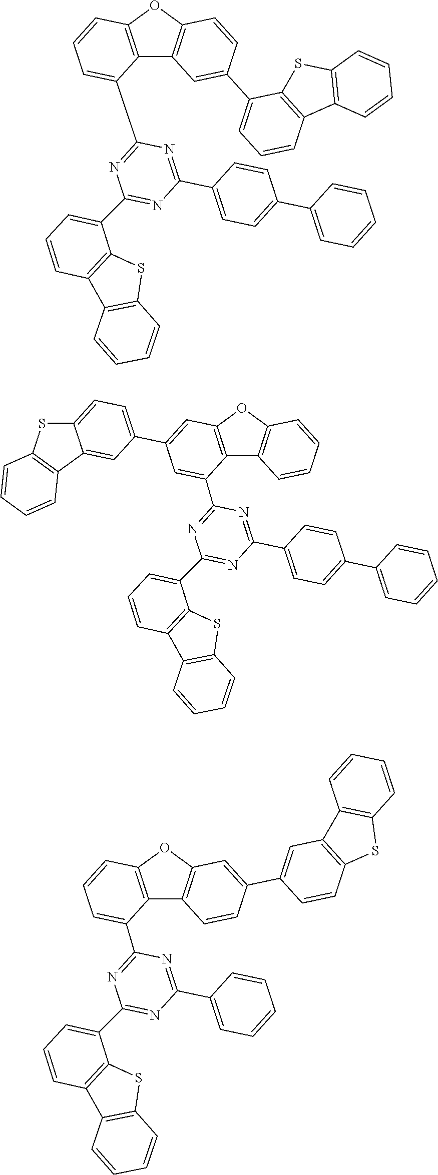

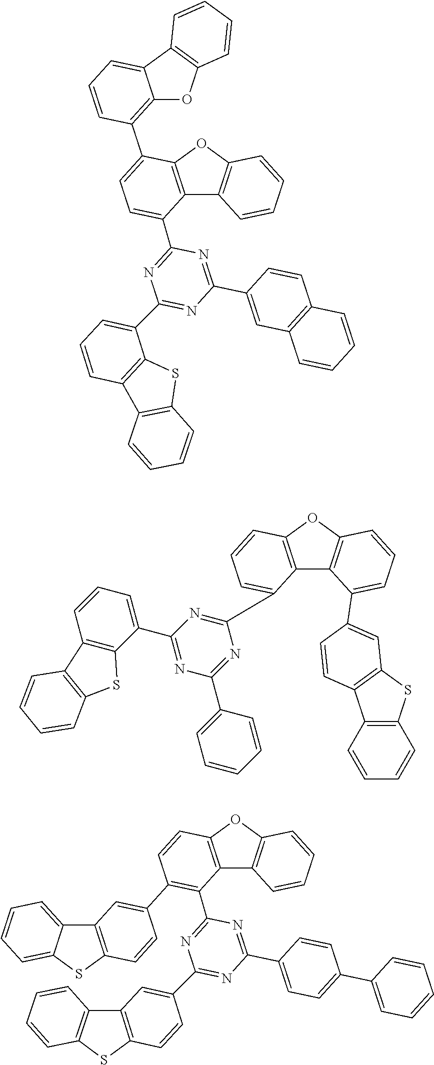

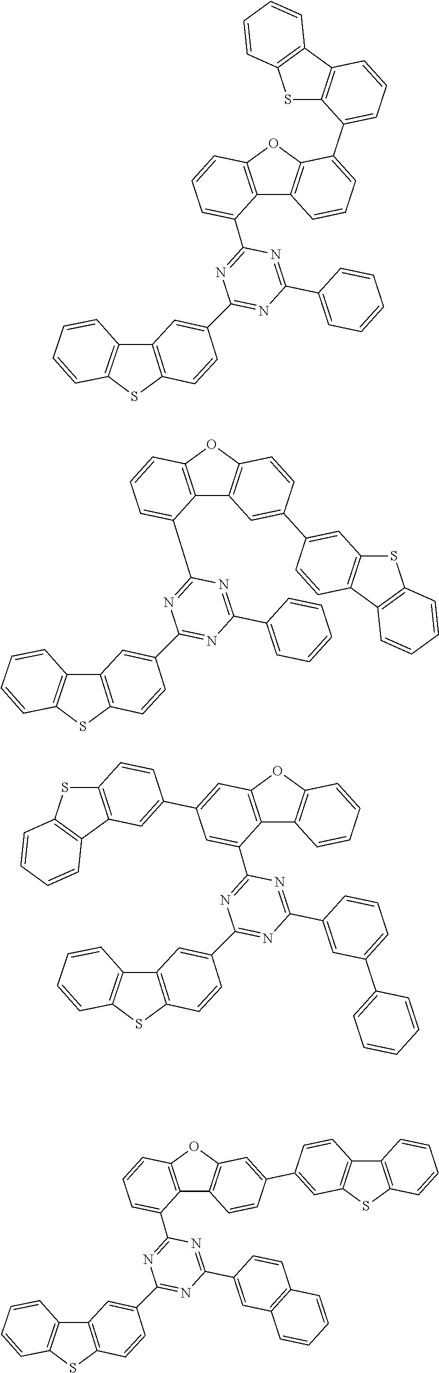 [SMILES: c1ccc(-c2nc(-c3ccc4sc5ccccc5c4c3)nc(-c3cccc4oc5c(-c6cccc7c6sc6ccccc67)cccc5c34)n2)cc1 ; c1ccc(-c2nc(-c3ccc4sc5ccccc5c4c3)nc(-c3cccc4oc5ccc(-c6ccc7c(c6)sc6ccccc67)cc5c34)n2)cc1 ; c1ccc(-c2cccc(-c3nc(-c4ccc5sc6ccccc6c5c4)nc(-c4cc(-c5ccc6sc7ccccc7c6c5)cc5oc6ccccc6c45)n3)c2)cc1 ; c1ccc2cc(-c3nc(-c4ccc5sc6ccccc6c5c4)nc(-c4cccc5oc6cc(-c7ccc8c(c7)sc7ccccc78)ccc6c45)n3)ccc2c1]

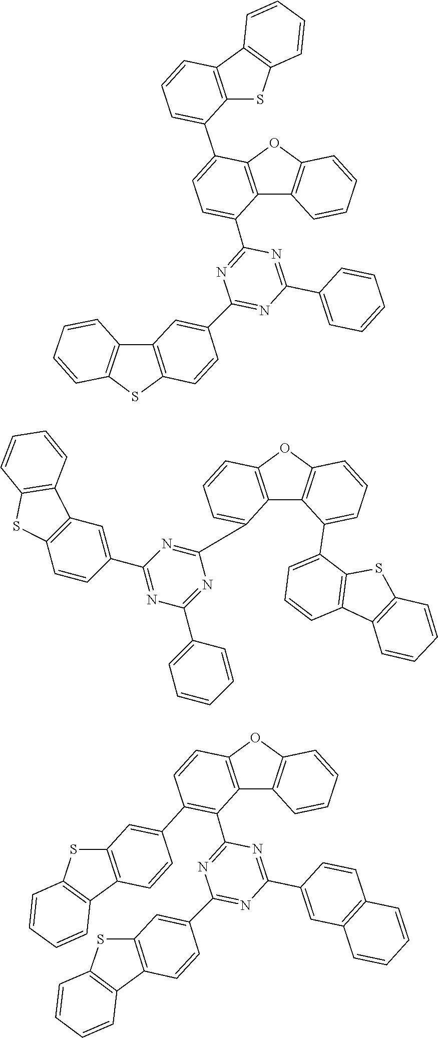 [SMILES: c1ccc(-c2nc(-c3ccc4sc5ccccc5c4c3)nc(-c3ccc(-c4cccc5c4sc4ccccc45)c4oc5ccccc5c34)n2)cc1 ; c1ccc(-c2nc(-c3ccc4sc5ccccc5c4c3)nc(-c3cccc4oc5cccc(-c6cccc7c6sc6ccccc67)c5c34)n2)cc1 ; c1ccc2cc(-c3nc(-c4ccc5c(c4)sc4ccccc45)nc(-c4c(-c5ccc6c(c5)sc5ccccc56)ccc5oc6ccccc6c45)n3)ccc2c1]

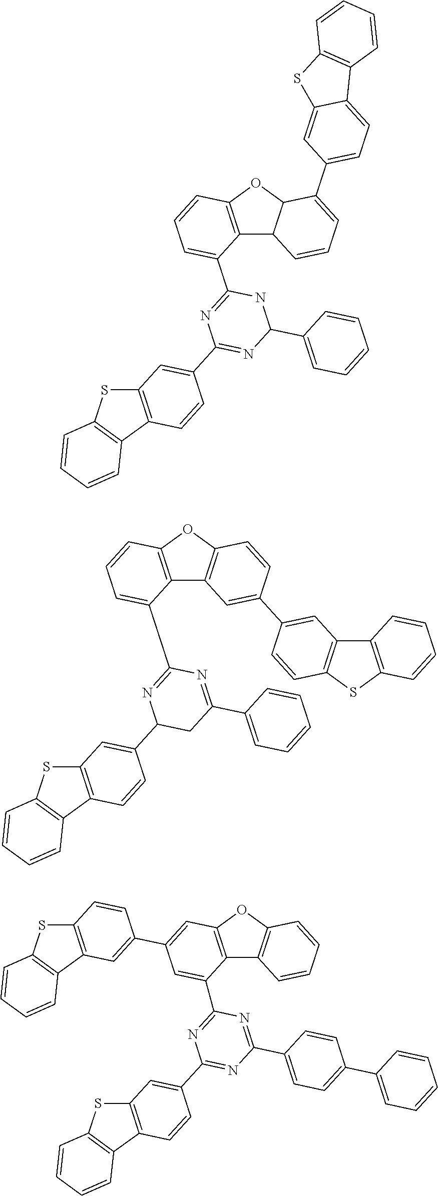

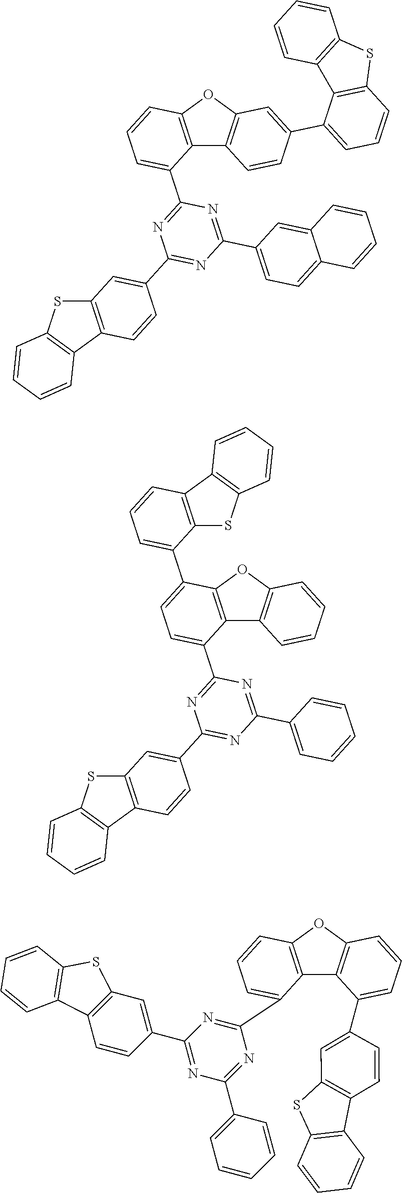 [SMILES: c1ccc2cc(-c3nc(-c4ccc5c(c4)sc4ccccc45)nc(-c4cccc5oc6cc(-c7cccc8sc9ccccc9c78)ccc6c45)n3)ccc2c1 ; c1ccc(-c2nc(-c3ccc4c(c3)sc3ccccc34)nc(-c3ccc(-c4cccc5c4sc4ccccc45)c4oc5ccccc5c34)n2)cc1 ; c1ccc(-c2nc(-c3ccc4c(c3)sc3ccccc34)nc(-c3cccc4oc5cccc(-c6ccc7c(c6)sc6ccccc67)c5c34)n2)cc1]

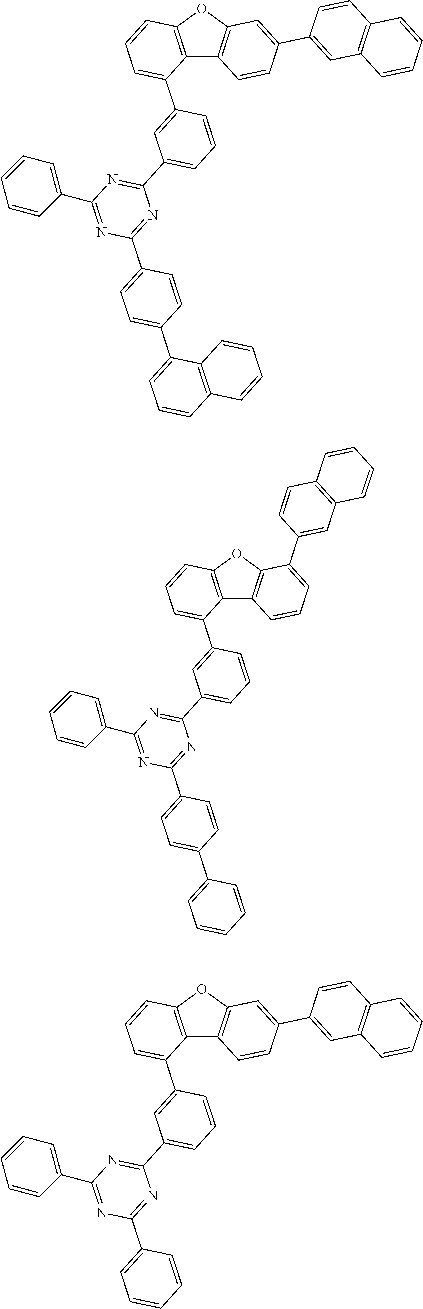 [SMILES: c1ccc(-c2nc(-c3ccc(-c4cccc5ccccc45)cc3)nc(-c3cccc(-c4cccc5oc6cc(-c7ccc8ccccc8c7)ccc6c45)c3)n2)cc1 ; c1ccc(-c2ccc(-c3nc(-c4ccccc4)nc(-c4cccc(-c5cccc6oc7c(-c8ccc9ccccc9c8)cccc7c56)c4)n3)cc2)cc1 ; c1ccc(-c2nc(-c3ccccc3)nc(-c3cccc(-c4cccc5oc6cc(-c7ccc8ccccc8c7)ccc6c45)c3)n2)cc1]

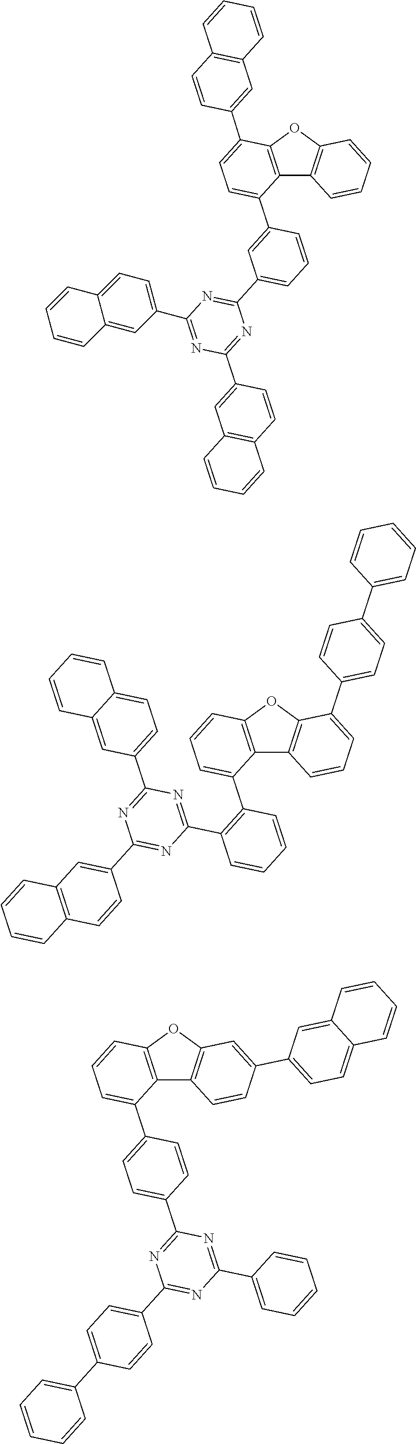 [SMILES: c1cc(-c2nc(-c3ccc4ccccc4c3)nc(-c3ccc4ccccc4c3)n2)cc(-c2ccc(-c3ccc4ccccc4c3)c3oc4ccccc4c23)c1 ; c1ccc(-c2ccc(-c3cccc4c3oc3cccc(-c5ccccc5-c5nc(-c6ccc7ccccc7c6)nc(-c6ccc7ccccc7c6)n5)c34)cc2)cc1 ; c1ccc(-c2ccc(-c3nc(-c4ccccc4)nc(-c4ccc(-c5cccc6oc7cc(-c8ccc9ccccc9c8)ccc7c56)cc4)n3)cc2)cc1]

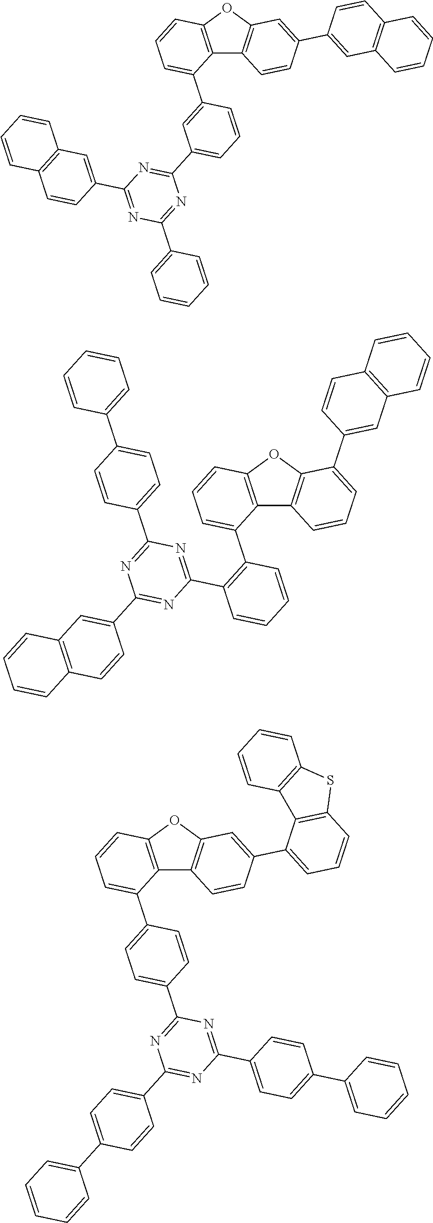

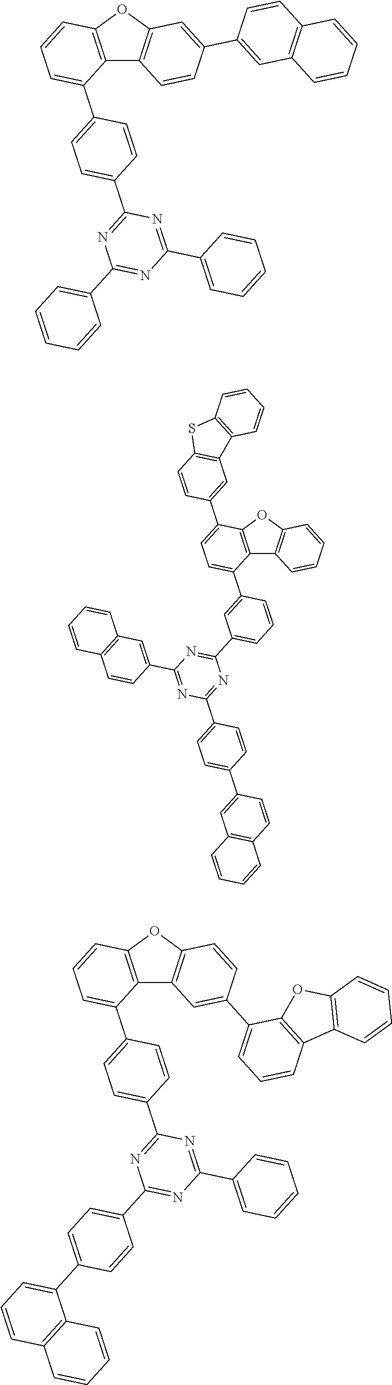

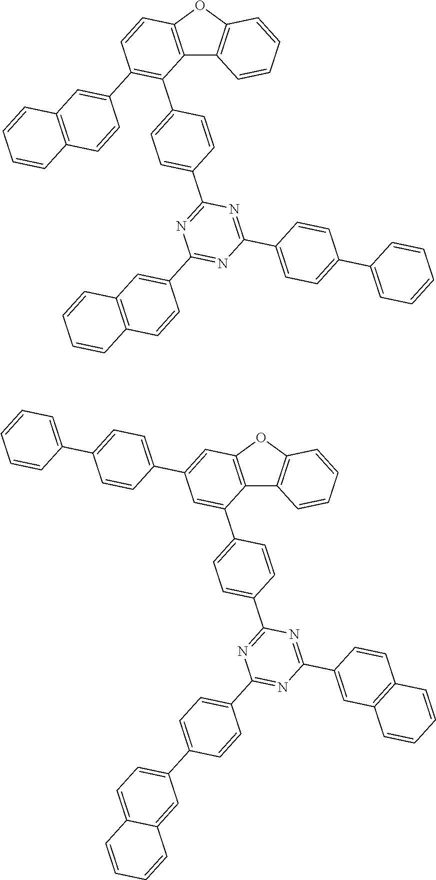

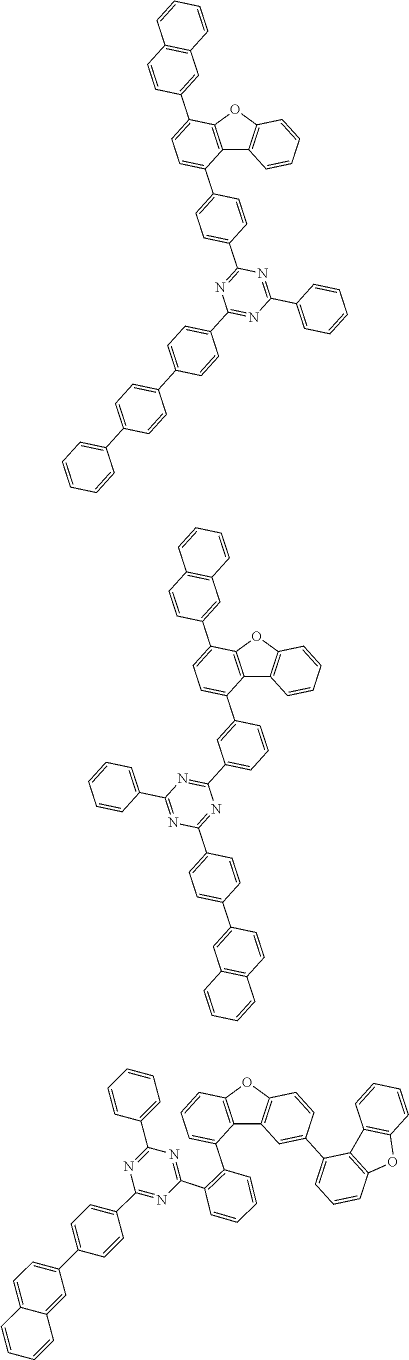

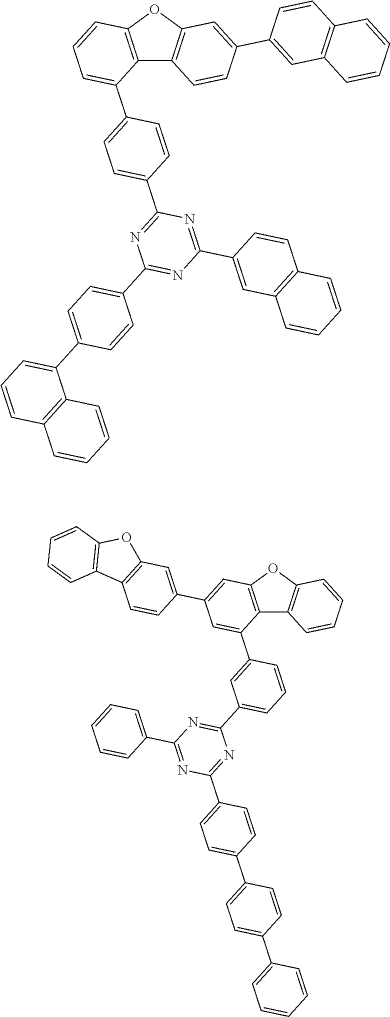 [SMILES: c1ccc2cc(-c3ccc4c(c3)oc3cccc(-c5ccc(-c6nc(-c7ccc(-c8cccc9ccccc89)cc7)nc(-c7ccc8ccccc8c7)n6)cc5)c34)ccc2c1 ; c1ccc(-c2ccc(-c3ccc(-c4nc(-c5ccccc5)nc(-c5cccc(-c6cc(-c7ccc8c(c7)oc7ccccc78)cc7oc8ccccc8c67)c5)n4)cc3)cc2)cc1]

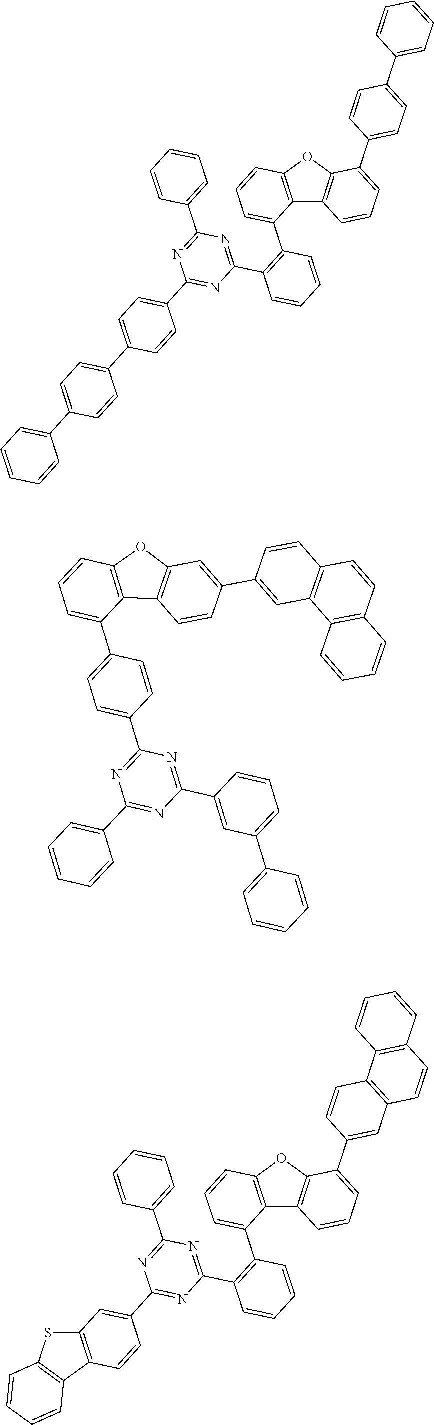

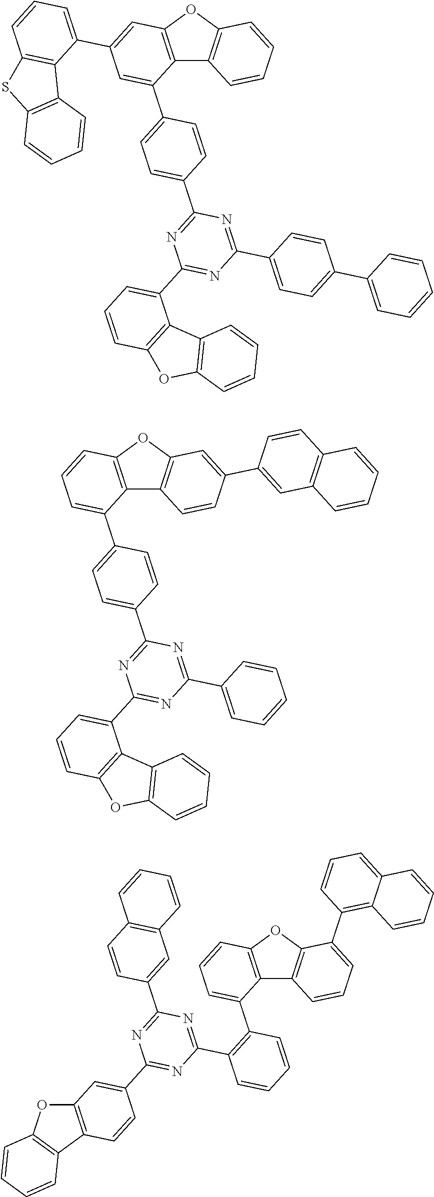 [SMILES: c1ccc(-c2ccc(-c3nc(-c4ccc(-c5cc(-c6cccc7sc8ccccc8c67)cc6oc7ccccc7c56)cc4)nc(-c4cccc5oc6ccccc6c45)n3)cc2)cc1 ; c1ccc(-c2nc(-c3ccc(-c4cccc5oc6cc(-c7ccc8ccccc8c7)ccc6c45)cc3)nc(-c3cccc4oc5ccccc5c34)n2)cc1 ; c1ccc(-c2cccc3oc4c(-c5cccc6ccccc56)cccc4c23)c(-c2nc(-c3ccc4ccccc4c3)nc(-c3ccc4c(c3)oc3ccccc34)n2)c1]

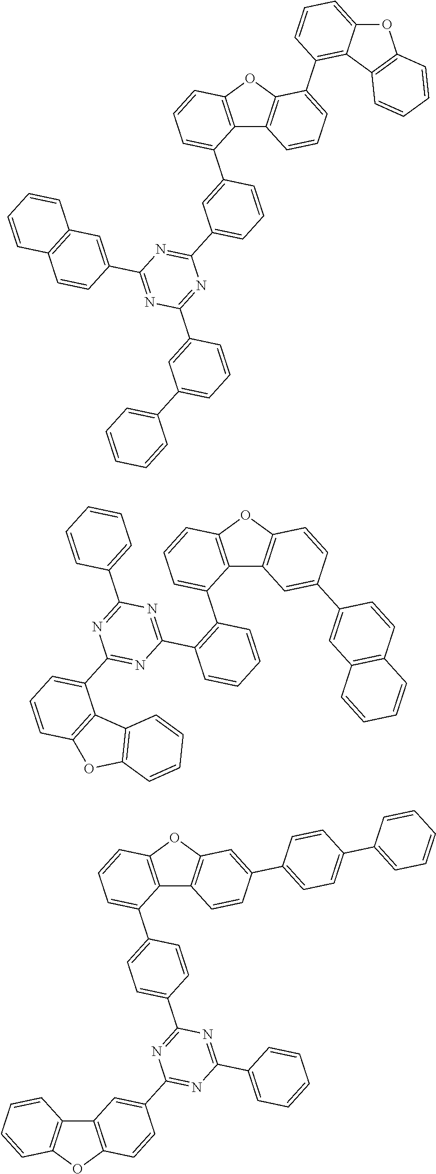 [SMILES: c1ccc(-c2cccc(-c3nc(-c4cccc(-c5cccc6oc7c(-c8cccc9oc%10ccccc%10c89)cccc7c56)c4)nc(-c4ccc5ccccc5c4)n3)c2)cc1 ; c1ccc(-c2nc(-c3ccccc3-c3cccc4oc5ccc(-c6ccc7ccccc7c6)cc5c34)nc(-c3cccc4oc5ccccc5c34)n2)cc1 ; c1ccc(-c2ccc(-c3ccc4c(c3)oc3cccc(-c5ccc(-c6nc(-c7ccccc7)nc(-c7ccc8oc9ccccc9c8c7)n6)cc5)c34)cc2)cc1]

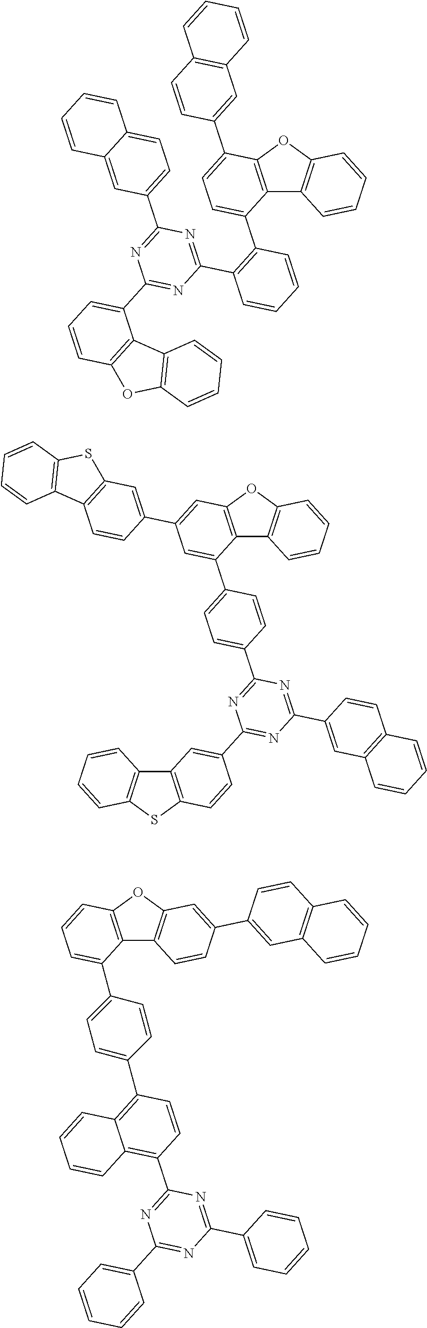 [SMILES: c1ccc(-c2ccc(-c3ccc4ccccc4c3)c3oc4ccccc4c23)c(-c2nc(-c3ccc4ccccc4c3)nc(-c3cccc4oc5ccccc5c34)n2)c1 ; c1ccc2cc(-c3nc(-c4ccc(-c5cc(-c6ccc7c(c6)sc6ccccc67)cc6oc7ccccc7c56)cc4)nc(-c4ccc5sc6ccccc6c5c4)n3)ccc2c1 ; c1ccc(-c2nc(-c3ccccc3)nc(-c3ccc(-c4ccc(-c5cccc6oc7cc(-c8ccc9ccccc9c8)ccc7c56)cc4)c4ccccc34)n2)cc1]

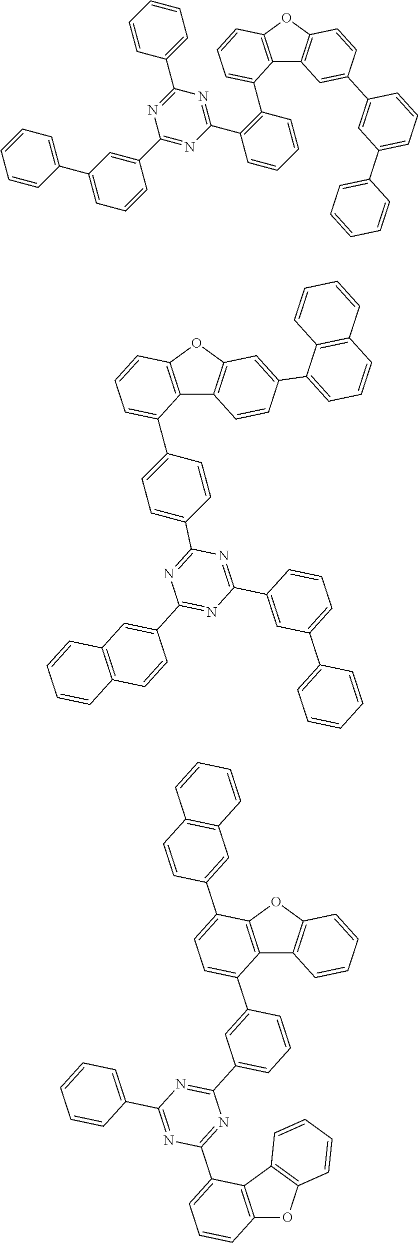

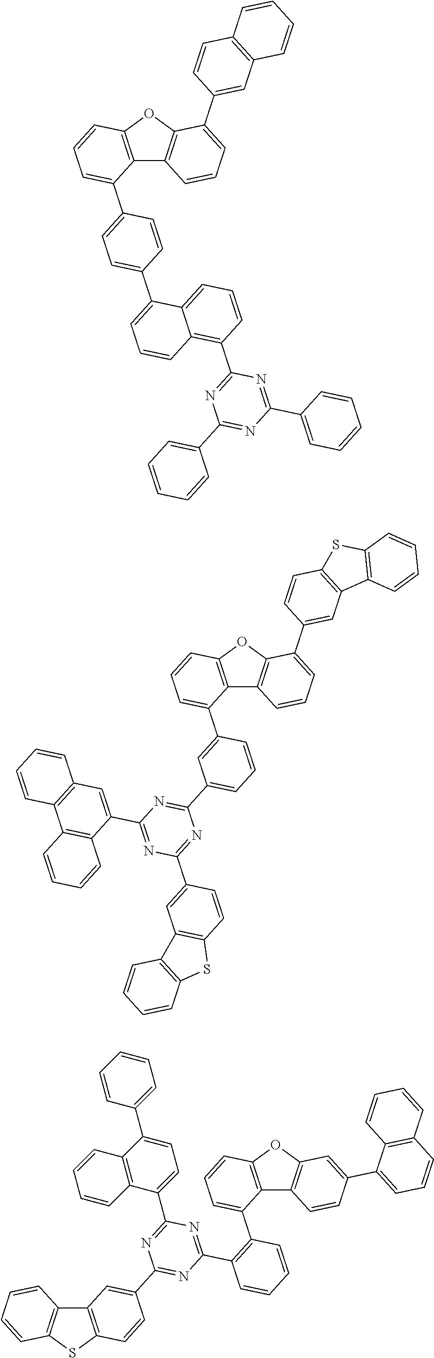 [SMILES: c1ccc(-c2nc(-c3ccccc3)nc(-c3cccc4c(-c5ccc(-c6cccc7oc8c(-c9ccc%10ccccc%10c9)cccc8c67)cc5)cccc34)n2)cc1 ; c1cc(-c2nc(-c3ccc4sc5ccccc5c4c3)nc(-c3cc4ccccc4c4ccccc34)n2)cc(-c2cccc3oc4c(-c5ccc6sc7ccccc7c6c5)cccc4c23)c1 ; c1ccc(-c2ccc(-c3nc(-c4ccc5sc6ccccc6c5c4)nc(-c4ccccc4-c4cccc5oc6cc(-c7cccc8ccccc78)ccc6c45)n3)c3ccccc23)cc1]

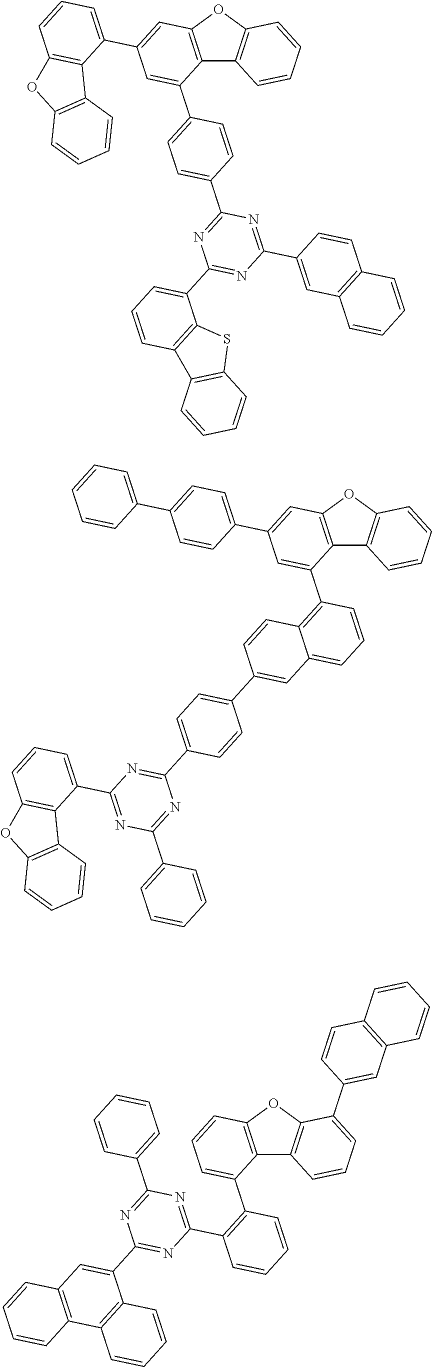 [SMILES: c1ccc2cc(-c3nc(-c4ccc(-c5cc(-c6cccc7oc8ccccc8c67)cc6oc7ccccc7c56)cc4)nc(-c4cccc5c4sc4ccccc45)n3)ccc2c1 ; c1ccc(-c2ccc(-c3cc(-c4cccc5cc(-c6ccc(-c7nc(-c8ccccc8)nc(-c8cccc9oc%10ccccc%10c89)n7)cc6)ccc45)c4c(c3)oc3ccccc34)cc2)cc1 ; c1ccc(-c2nc(-c3ccccc3-c3cccc4oc5c(-c6ccc7ccccc7c6)cccc5c34)nc(-c3cc4ccccc4c4ccccc34)n2)cc1]

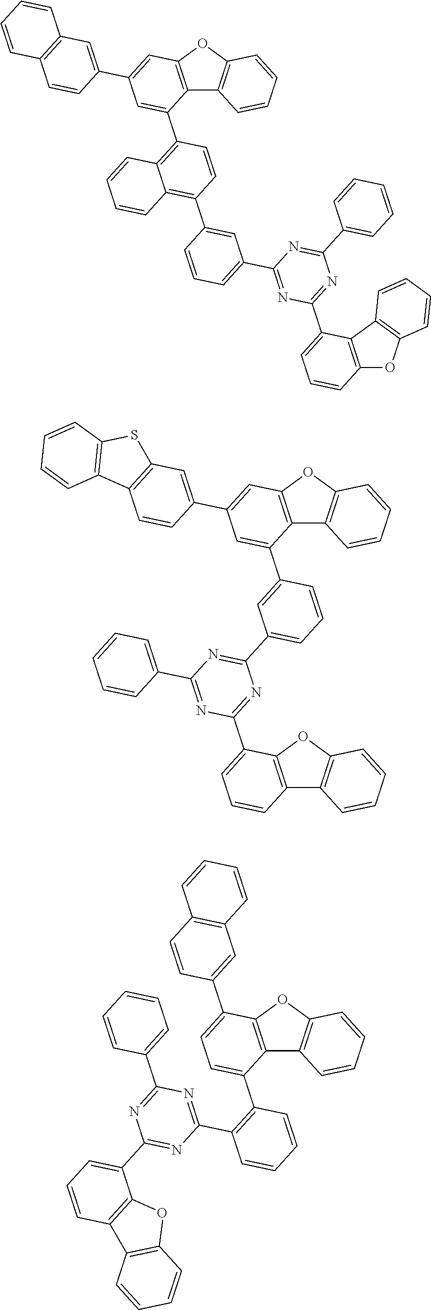 [SMILES: c1ccc(-c2nc(-c3cccc(-c4ccc(-c5cc(-c6ccc7ccccc7c6)cc6oc7ccccc7c56)c5ccccc45)c3)nc(-c3cccc4oc5ccccc5c34)n2)cc1 ; c1ccc(-c2nc(-c3cccc(-c4cc(-c5ccc6c(c5)sc5ccccc56)cc5oc6ccccc6c45)c3)nc(-c3cccc4c3oc3ccccc34)n2)cc1 ; c1ccc(-c2nc(-c3ccccc3-c3ccc(-c4ccc5ccccc5c4)c4oc5ccccc5c34)nc(-c3cccc4c3oc3ccccc34)n2)cc1]

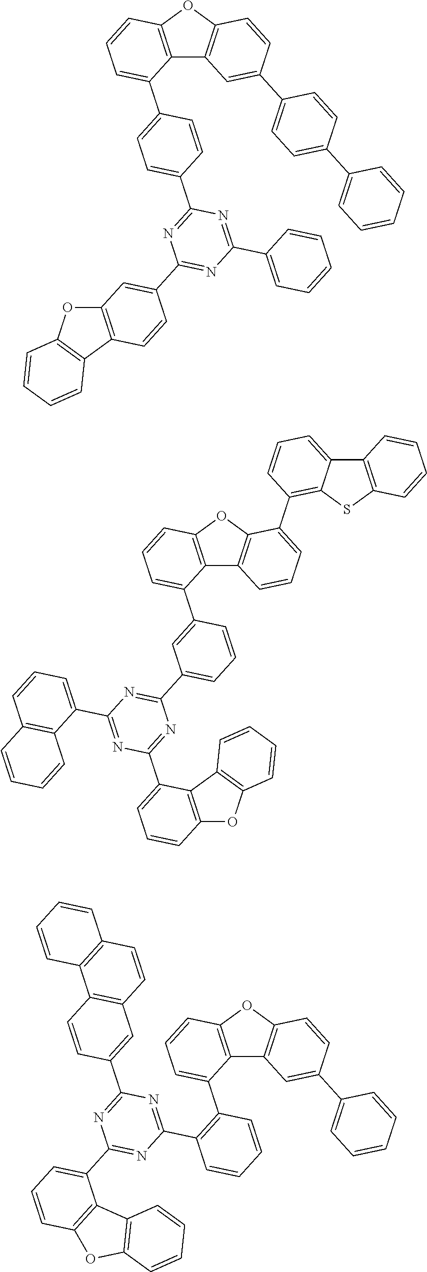 [SMILES: c1ccc(-c2ccc(-c3ccc4oc5cccc(-c6ccc(-c7nc(-c8ccccc8)nc(-c8ccc9c(c8)oc8ccccc89)n7)cc6)c5c4c3)cc2)cc1 ; c1cc(-c2nc(-c3cccc4ccccc34)nc(-c3cccc4oc5ccccc5c34)n2)cc(-c2cccc3oc4c(-c5cccc6c5sc5ccccc56)cccc4c23)c1 ; c1ccc(-c2ccc3oc4cccc(-c5ccccc5-c5nc(-c6ccc7c(ccc8ccccc87)c6)nc(-c6cccc7oc8ccccc8c67)n5)c4c3c2)cc1]

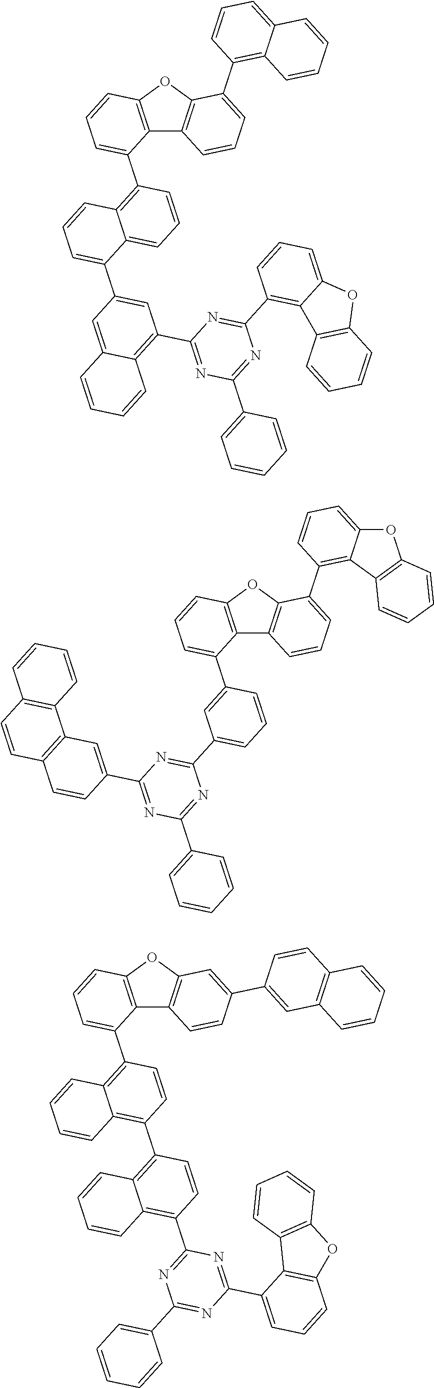 [SMILES: c1ccc(-c2nc(-c3cc(-c4cccc5c(-c6cccc7oc8c(-c9cccc%10ccccc9%10)cccc8c67)cccc45)cc4ccccc34)nc(-c3cccc4oc5ccccc5c34)n2)cc1 ; c1ccc(-c2nc(-c3cccc(-c4cccc5oc6c(-c7cccc8oc9ccccc9c78)cccc6c45)c3)nc(-c3ccc4ccc5ccccc5c4c3)n2)cc1 ; c1ccc(-c2nc(-c3ccc(-c4ccc(-c5cccc6oc7cc(-c8ccc9ccccc9c8)ccc7c56)c5ccccc45)c4ccccc34)nc(-c3cccc4oc5ccccc5c34)n2)cc1]

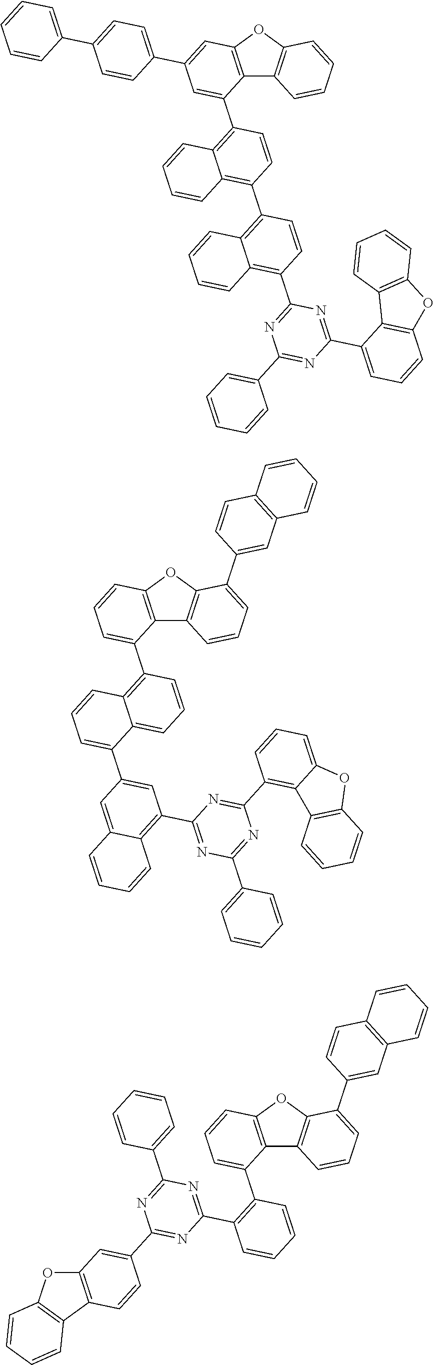

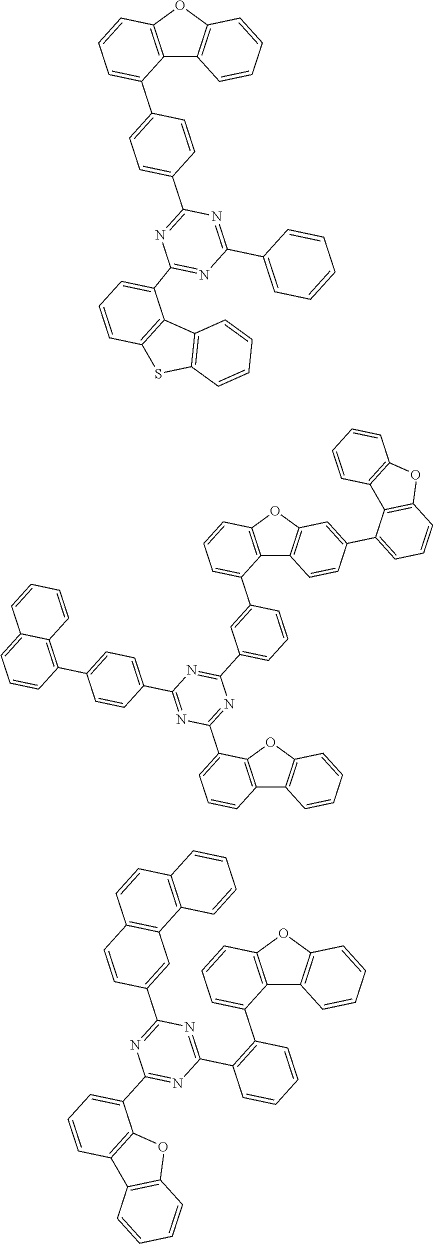 [SMILES: c1ccc(-c2nc(-c3ccc(-c4cccc5oc6ccccc6c45)cc3)nc(-c3cccc4sc5ccccc5c34)n2)cc1 ; c1cc(-c2nc(-c3ccc(-c4cccc5ccccc45)cc3)nc(-c3cccc4c3oc3ccccc34)n2)cc(-c2cccc3oc4cc(-c5cccc6oc7ccccc7c56)ccc4c23)c1 ; c1ccc(-c2cccc3oc4ccccc4c23)c(-c2nc(-c3ccc4ccc5ccccc5c4c3)nc(-c3cccc4c3oc3ccccc34)n2)c1]

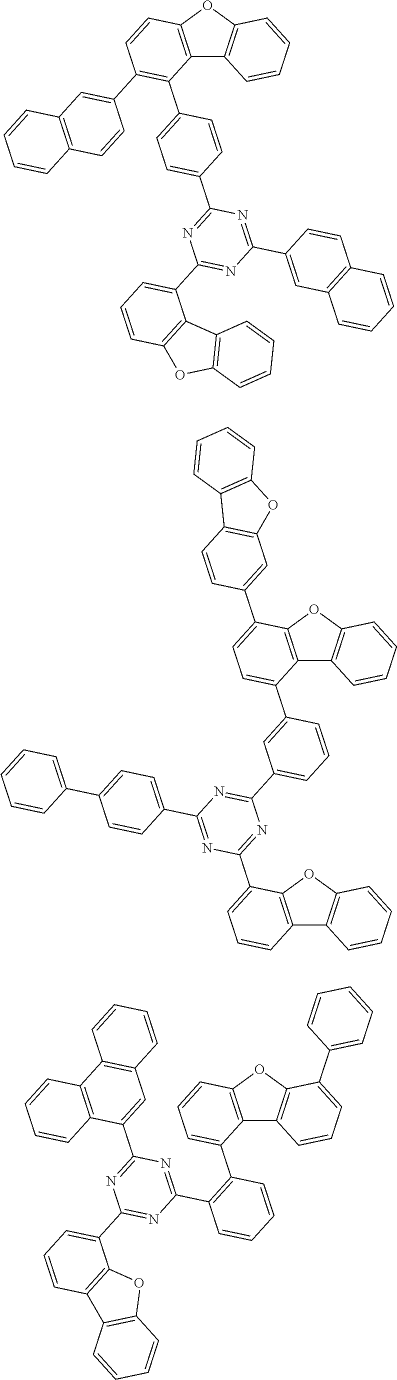

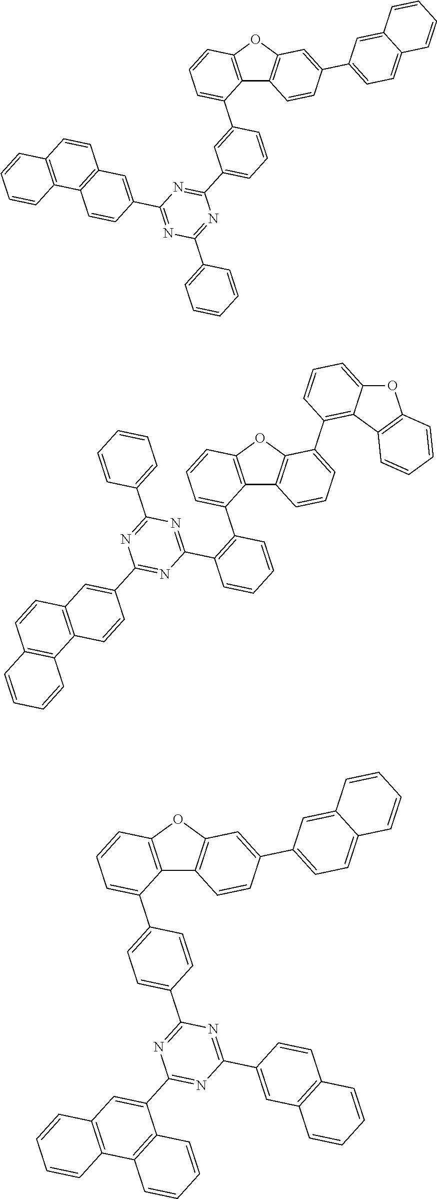 [SMILES: c1ccc(-c2nc(-c3cccc(-c4cccc5oc6cc(-c7ccc8ccccc8c7)ccc6c45)c3)nc(-c3ccc4c(ccc5ccccc54)c3)n2)cc1 ; c1ccc(-c2nc(-c3ccc4c(ccc5ccccc54)c3)nc(-c3ccccc3-c3cccc4oc5c(-c6cccc7oc8ccccc8c67)cccc5c34)n2)cc1 ; c1ccc2cc(-c3ccc4c(c3)oc3cccc(-c5ccc(-c6nc(-c7ccc8ccccc8c7)nc(-c7cc8ccccc8c8ccccc78)n6)cc5)c34)ccc2c1]

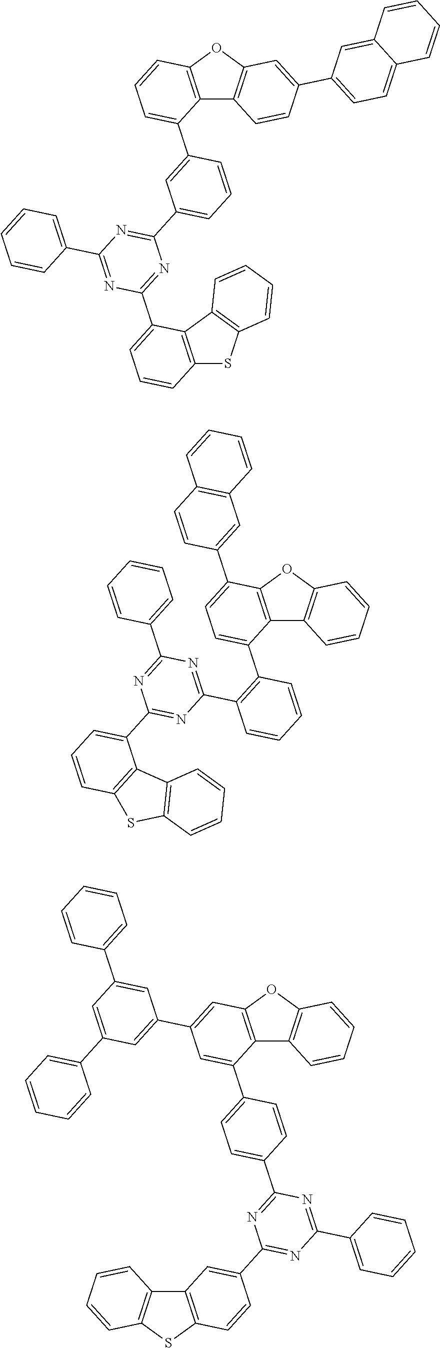 [SMILES: c1ccc(-c2nc(-c3cccc(-c4cccc5oc6cc(-c7ccc8ccccc8c7)ccc6c45)c3)nc(-c3cccc4sc5ccccc5c34)n2)cc1 ; c1ccc(-c2nc(-c3ccccc3-c3ccc(-c4ccc5ccccc5c4)c4oc5ccccc5c34)nc(-c3cccc4sc5ccccc5c34)n2)cc1 ; c1ccc(-c2cc(-c3ccccc3)cc(-c3cc(-c4ccc(-c5nc(-c6ccccc6)nc(-c6ccc7sc8ccccc8c7c6)n5)cc4)c4c(c3)oc3ccccc34)c2)cc1]

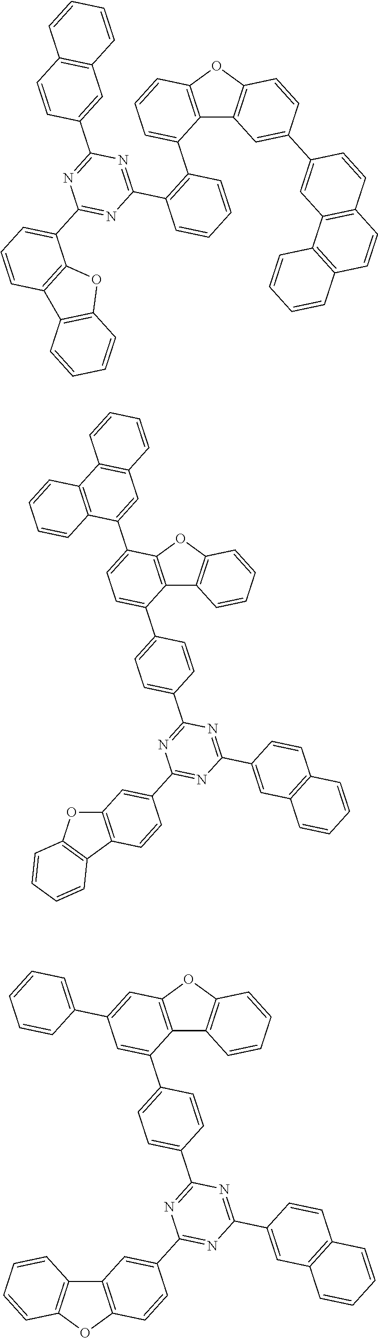

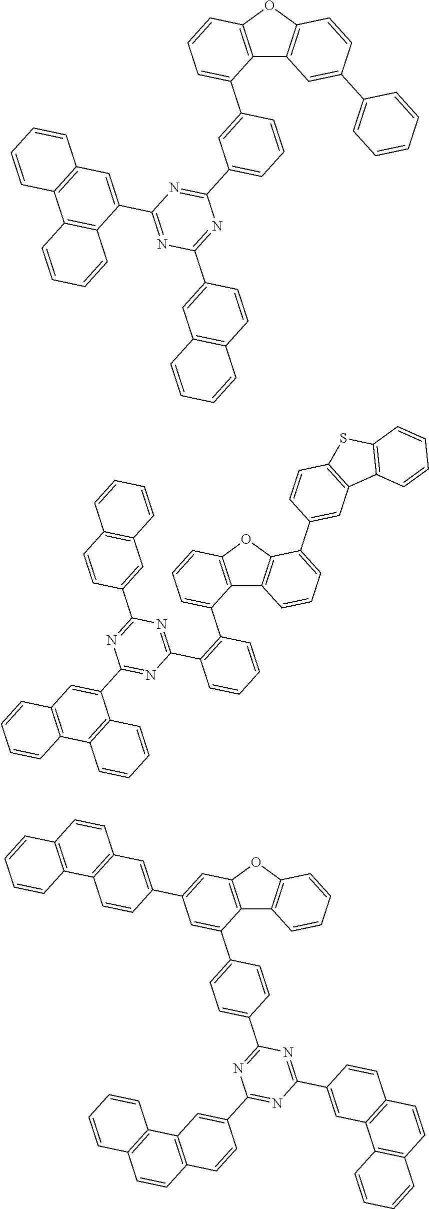 [SMILES: c1ccc(-c2ccc3oc4cccc(-c5cccc(-c6nc(-c7ccc8ccccc8c7)nc(-c7cc8ccccc8c8ccccc78)n6)c5)c4c3c2)cc1 ; c1ccc(-c2cccc3oc4c(-c5ccc6sc7ccccc7c6c5)cccc4c23)c(-c2nc(-c3ccc4ccccc4c3)nc(-c3cc4ccccc4c4ccccc34)n2)c1 ; c1ccc2c(c1)ccc1cc(-c3cc(-c4ccc(-c5nc(-c6ccc7ccc8ccccc8c7c6)nc(-c6ccc7ccc8ccccc8c7c6)n5)cc4)c4c(c3)oc3ccccc34)ccc12]

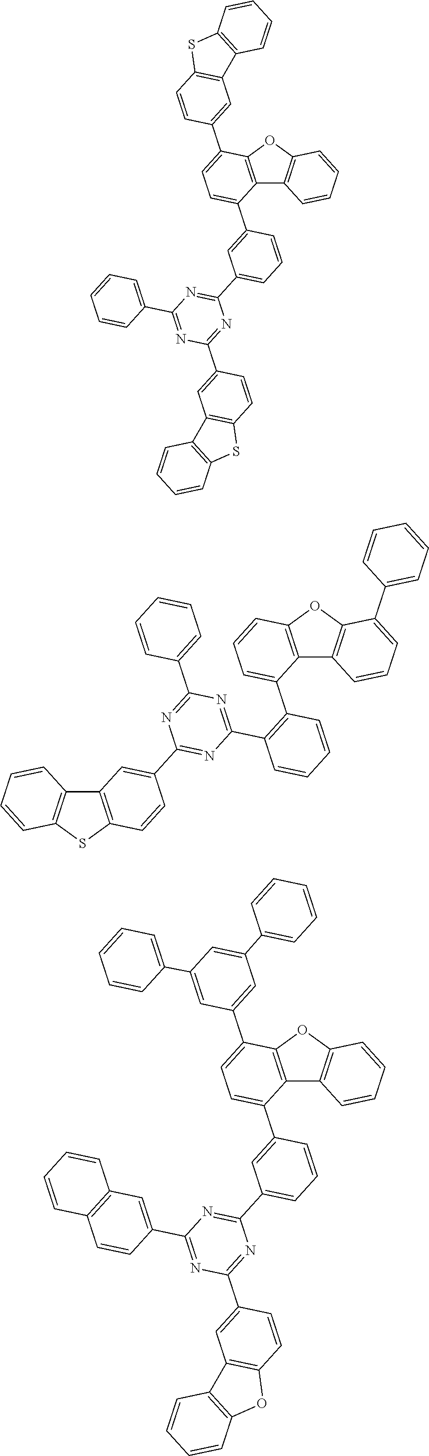 [SMILES: c1ccc(-c2nc(-c3cccc(-c4ccc(-c5ccc6sc7ccccc7c6c5)c5oc6ccccc6c45)c3)nc(-c3ccc4sc5ccccc5c4c3)n2)cc1 ; c1ccc(-c2nc(-c3ccc4sc5ccccc5c4c3)nc(-c3ccccc3-c3cccc4oc5c(-c6ccccc6)cccc5c34)n2)cc1 ; c1ccc(-c2cc(-c3ccccc3)cc(-c3ccc(-c4cccc(-c5nc(-c6ccc7ccccc7c6)nc(-c6ccc7oc8ccccc8c7c6)n5)c4)c4c3oc3ccccc34)c2)cc1]

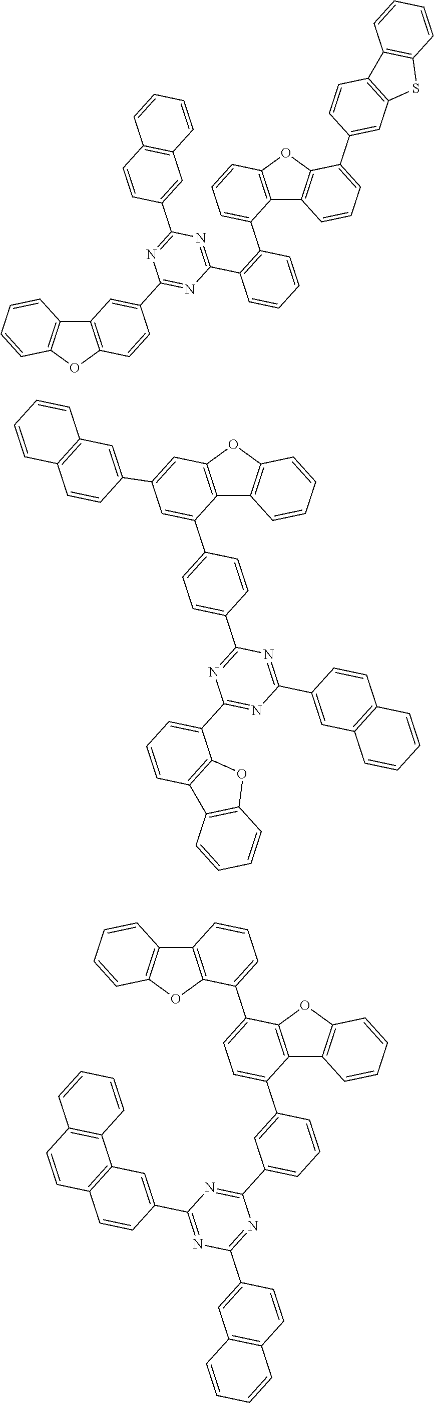

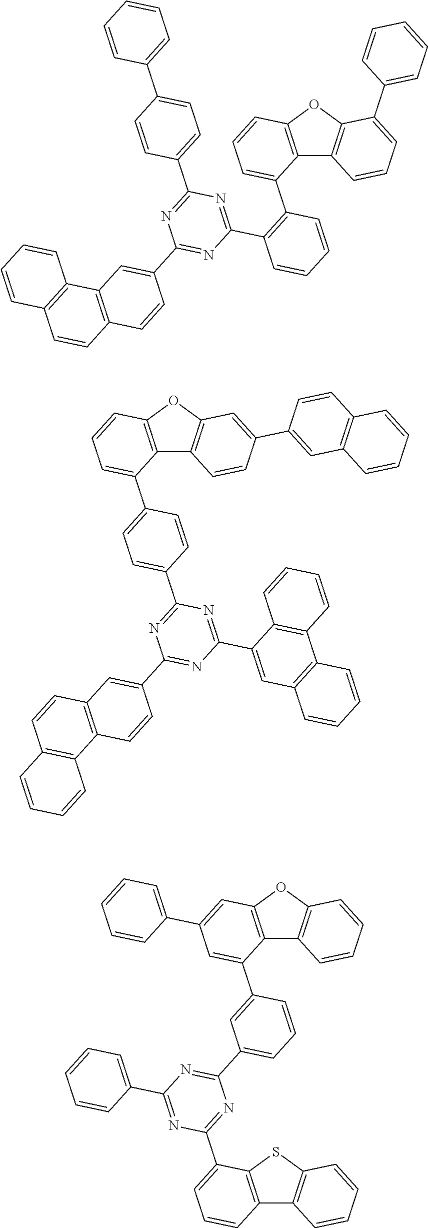 [SMILES: c1ccc(-c2ccc(-c3nc(-c4ccc5ccc6ccccc6c5c4)nc(-c4ccccc4-c4cccc5oc6c(-c7ccccc7)cccc6c45)n3)cc2)cc1 ; c1ccc2cc(-c3ccc4c(c3)oc3cccc(-c5ccc(-c6nc(-c7ccc8c(ccc9ccccc98)c7)nc(-c7cc8ccccc8c8ccccc78)n6)cc5)c34)ccc2c1 ; c1ccc(-c2cc(-c3cccc(-c4nc(-c5ccccc5)nc(-c5cccc6c5sc5ccccc56)n4)c3)c3c(c2)oc2ccccc23)cc1]

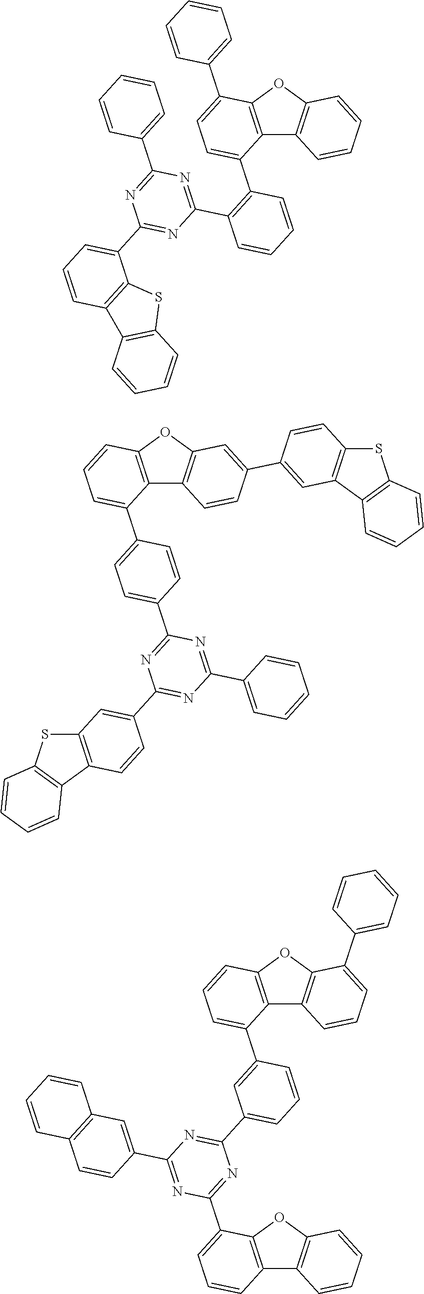

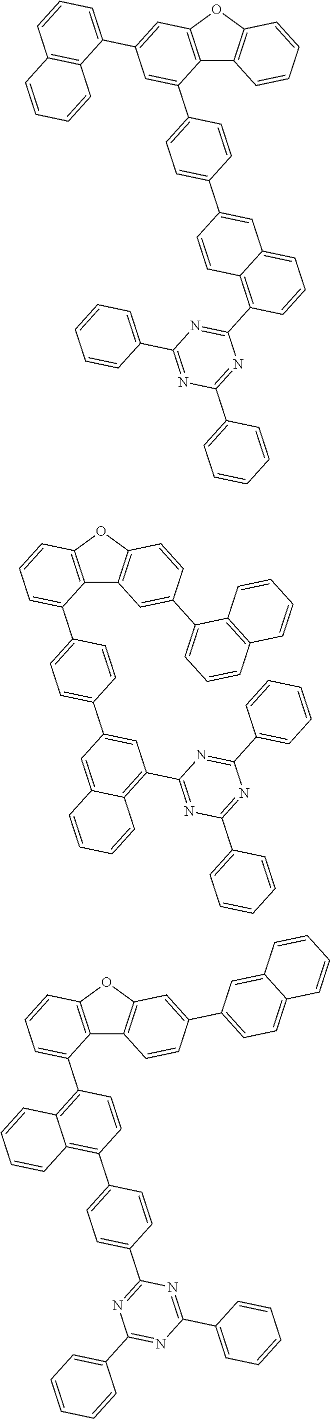

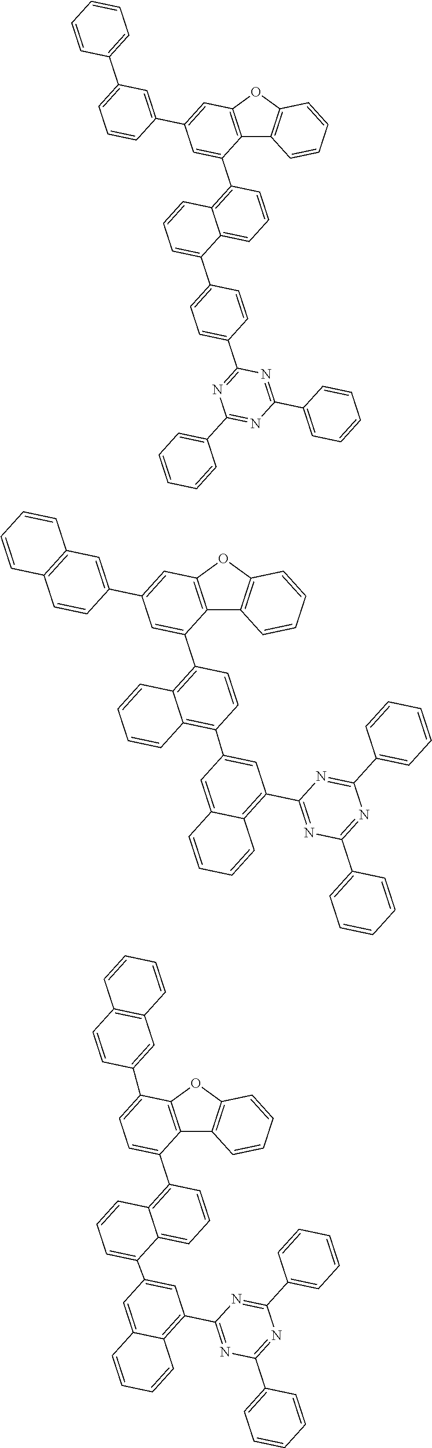 [SMILES: c1ccc(-c2cccc(-c3cc(-c4cccc5c(-c6ccc(-c7nc(-c8ccccc8)nc(-c8ccccc8)n7)cc6)cccc45)c4c(c3)oc3ccccc34)c2)cc1 ; c1ccc(-c2nc(-c3ccccc3)nc(-c3cc(-c4ccc(-c5cc(-c6ccc7ccccc7c6)cc6oc7ccccc7c56)c5ccccc45)cc4ccccc34)n2)cc1 ; c1ccc(-c2nc(-c3ccccc3)nc(-c3cc(-c4cccc5c(-c6ccc(-c7ccc8ccccc8c7)c7oc8ccccc8c67)cccc45)cc4ccccc34)n2)cc1]

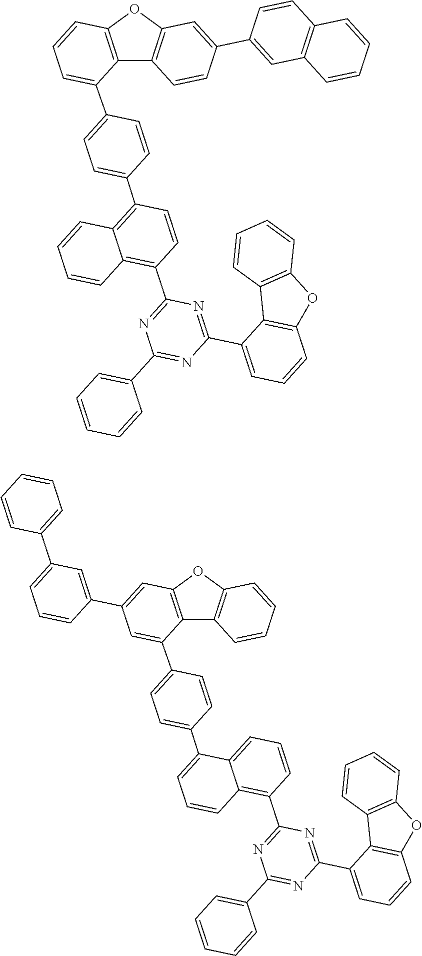 [SMILES: c1ccc(-c2nc(-c3ccc(-c4ccc(-c5cccc6oc7cc(-c8ccc9ccccc9c8)ccc7c56)cc4)c4ccccc34)nc(-c3cccc4oc5ccccc5c34)n2)cc1 ; c1ccc(-c2cccc(-c3cc(-c4ccc(-c5cccc6c(-c7nc(-c8ccccc8)nc(-c8cccc9oc%10ccccc%10c89)n7)cccc56)cc4)c4c(c3)oc3ccccc34)c2)cc1]

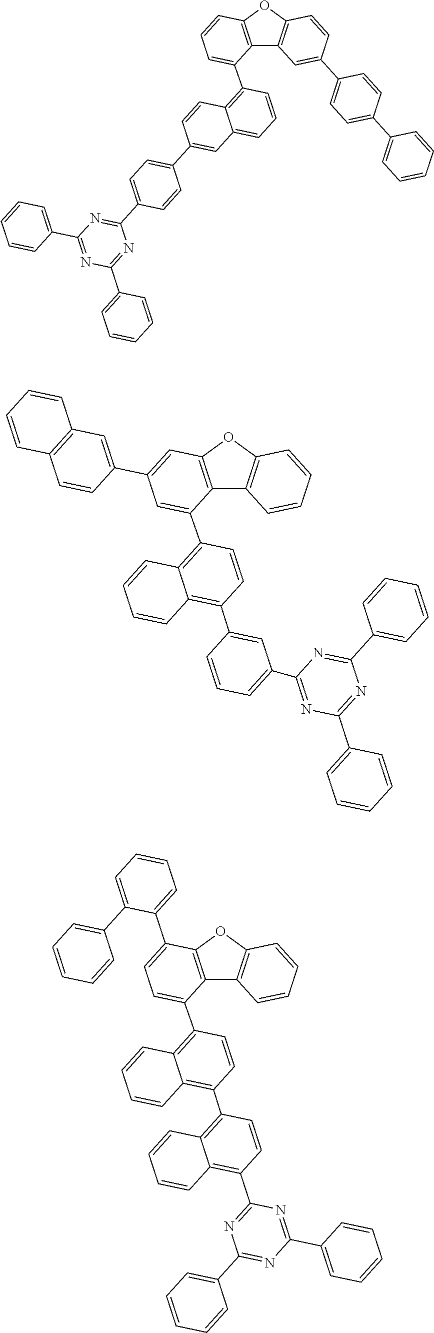 [SMILES: c1ccc(-c2ccc(-c3ccc4oc5cccc(-c6cccc7cc(-c8ccc(-c9nc(-c%10ccccc%10)nc(-c%10ccccc%10)n9)cc8)ccc67)c5c4c3)cc2)cc1 ; c1ccc(-c2nc(-c3ccccc3)nc(-c3cccc(-c4ccc(-c5cc(-c6ccc7ccccc7c6)cc6oc7ccccc7c56)c5ccccc45)c3)n2)cc1 ; c1ccc(-c2nc(-c3ccccc3)nc(-c3ccc(-c4ccc(-c5ccc(-c6ccccc6-c6ccccc6)c6oc7ccccc7c56)c5ccccc45)c4ccccc34)n2)cc1]

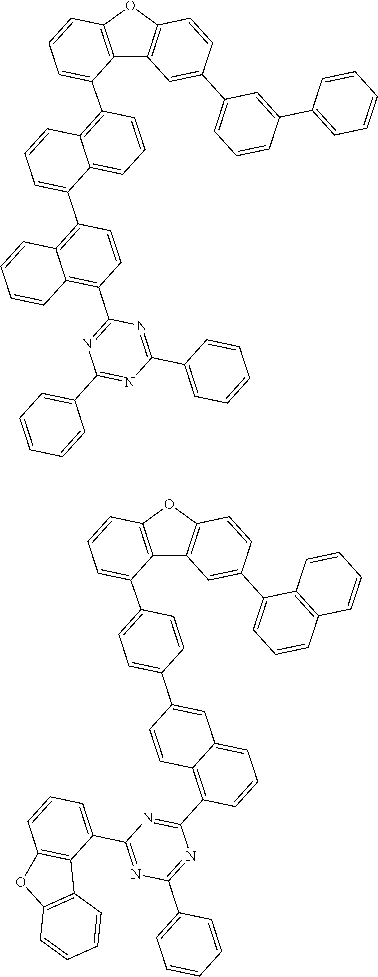 [SMILES: c1ccc(-c2cccc(-c3ccc4oc5cccc(-c6cccc7c(-c8ccc(-c9nc(-c%10ccccc%10)nc(-c%10ccccc%10)n9)c9ccccc89)cccc67)c5c4c3)c2)cc1 ; c1ccc(-c2nc(-c3cccc4cc(-c5ccc(-c6cccc7oc8ccc(-c9cccc%10ccccc9%10)cc8c67)cc5)ccc34)nc(-c3cccc4oc5ccccc5c34)n2)cc1]

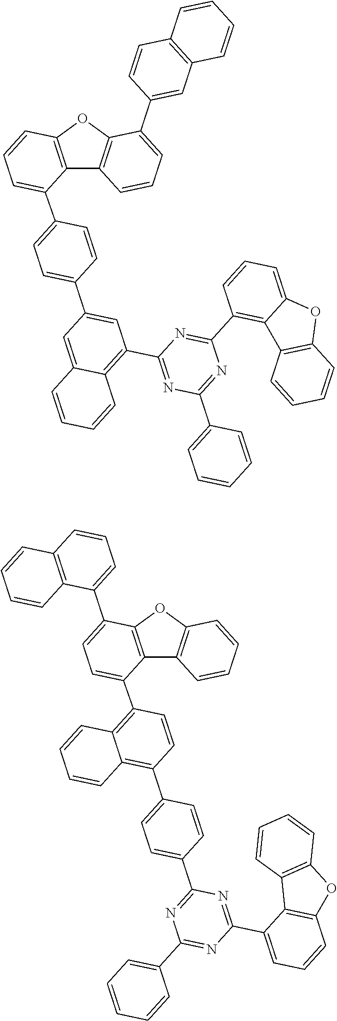 [SMILES: c1ccc(-c2nc(-c3cc(-c4ccc(-c5cccc6oc7c(-c8ccc9ccccc9c8)cccc7c56)cc4)cc4ccccc34)nc(-c3cccc4oc5ccccc5c34)n2)cc1 ; c1ccc(-c2nc(-c3ccc(-c4ccc(-c5ccc(-c6cccc7ccccc67)c6oc7ccccc7c56)c5ccccc45)cc3)nc(-c3cccc4oc5ccccc5c34)n2)cc1]

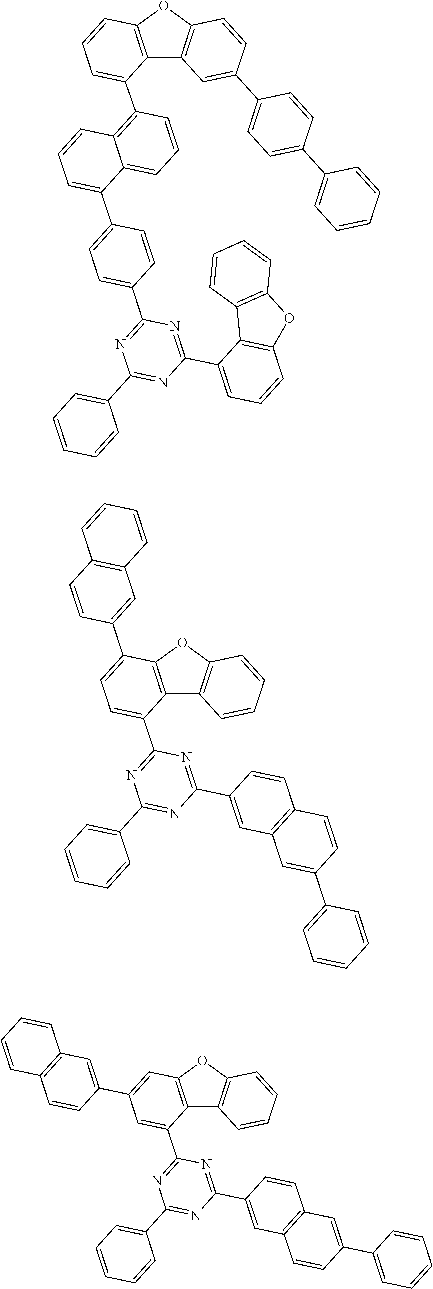 [SMILES: c1ccc(-c2ccc(-c3ccc4oc5cccc(-c6cccc7c(-c8ccc(-c9nc(-c%10ccccc%10)nc(-c%10cccc%11oc%12ccccc%12c%10%11)n9)cc8)cccc67)c5c4c3)cc2)cc1 ; c1ccc(-c2ccc3ccc(-c4nc(-c5ccccc5)nc(-c5ccc(-c6ccc7ccccc7c6)c6oc7ccccc7c56)n4)cc3c2)cc1 ; c1ccc(-c2ccc3cc(-c4nc(-c5ccccc5)nc(-c5cc(-c6ccc7ccccc7c6)cc6oc7ccccc7c56)n4)ccc3c2)cc1]

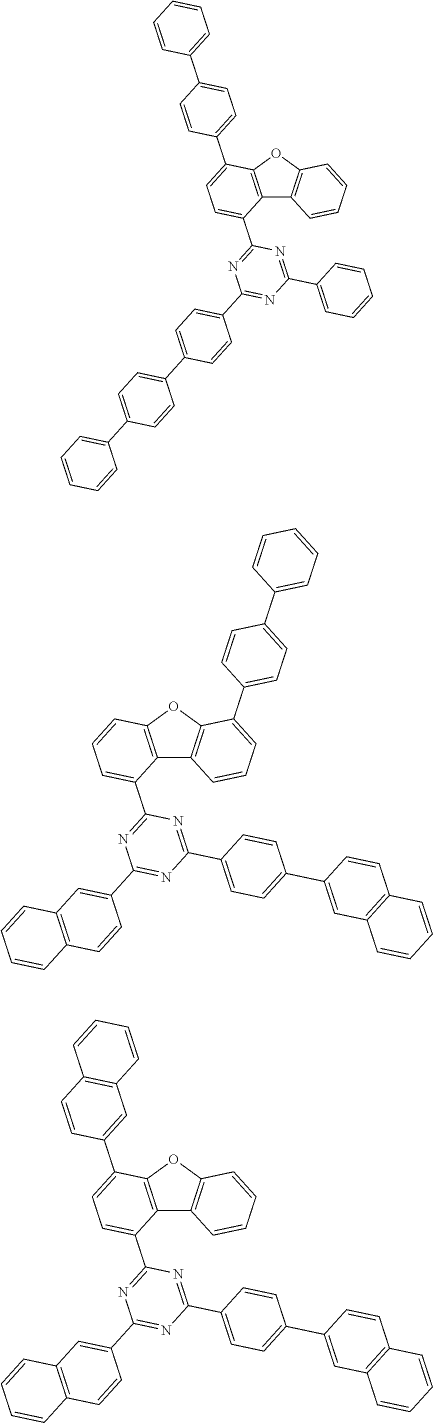 [SMILES: c1ccc(-c2ccc(-c3ccc(-c4nc(-c5ccccc5)nc(-c5ccc(-c6ccc(-c7ccccc7)cc6)c6oc7ccccc7c56)n4)cc3)cc2)cc1 ; c1ccc(-c2ccc(-c3cccc4c3oc3cccc(-c5nc(-c6ccc(-c7ccc8ccccc8c7)cc6)nc(-c6ccc7ccccc7c6)n5)c34)cc2)cc1 ; c1ccc2cc(-c3ccc(-c4nc(-c5ccc6ccccc6c5)nc(-c5ccc(-c6ccc7ccccc7c6)c6oc7ccccc7c56)n4)cc3)ccc2c1]

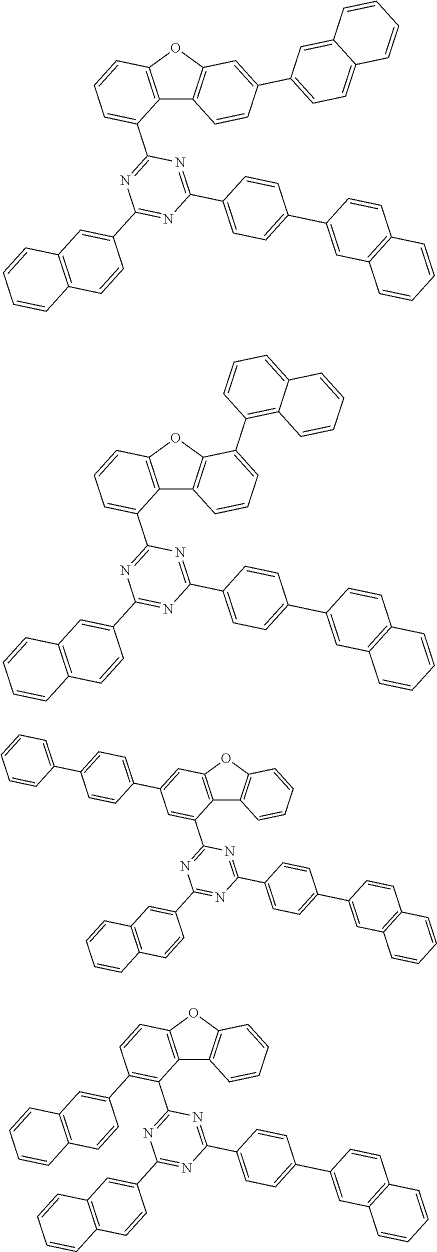

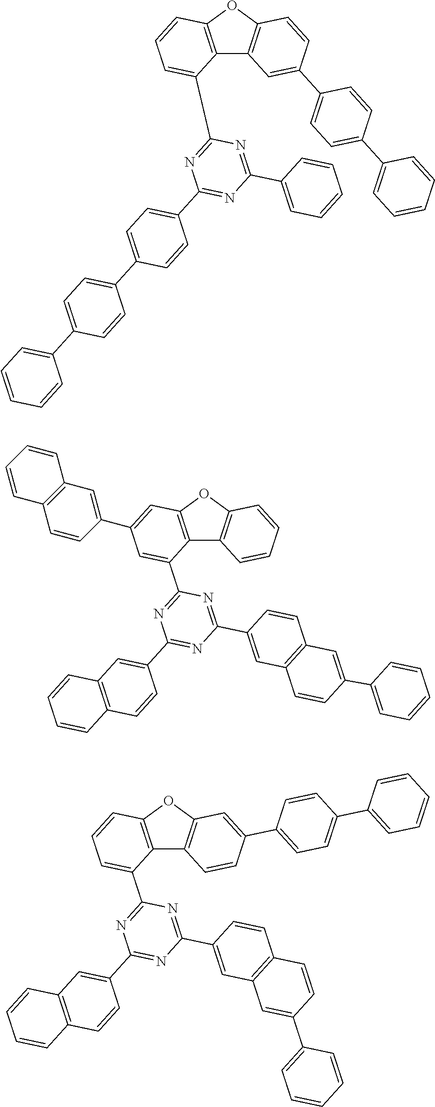

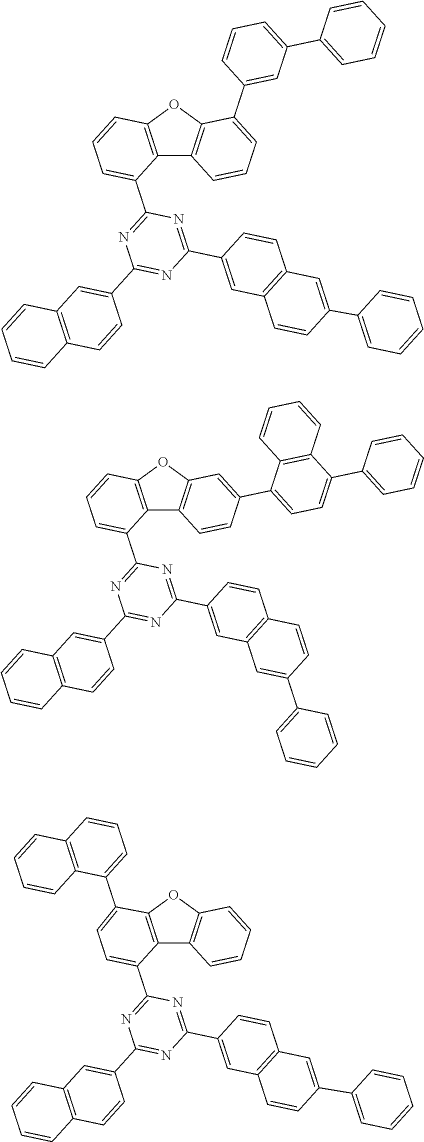 [SMILES: c1ccc(-c2cccc(-c3cccc4c3oc3cccc(-c5nc(-c6ccc7ccccc7c6)nc(-c6ccc7cc(-c8ccccc8)ccc7c6)n5)c34)c2)cc1 ; c1ccc(-c2ccc3ccc(-c4nc(-c5ccc6ccccc6c5)nc(-c5cccc6oc7cc(-c8ccc(-c9ccccc9)c9ccccc89)ccc7c56)n4)cc3c2)cc1 ; c1ccc(-c2ccc3cc(-c4nc(-c5ccc6ccccc6c5)nc(-c5ccc(-c6cccc7ccccc67)c6oc7ccccc7c56)n4)ccc3c2)cc1]

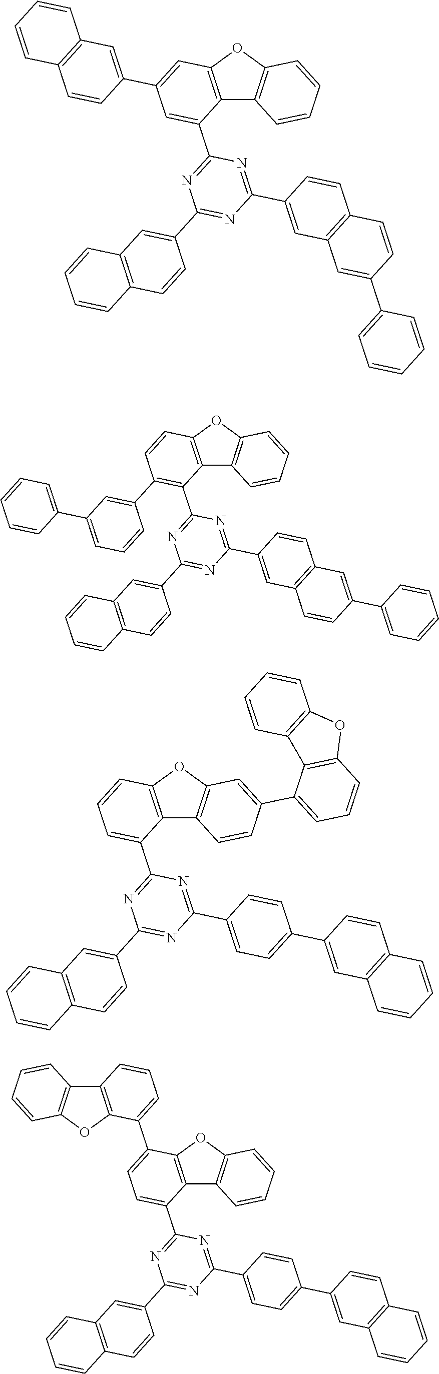

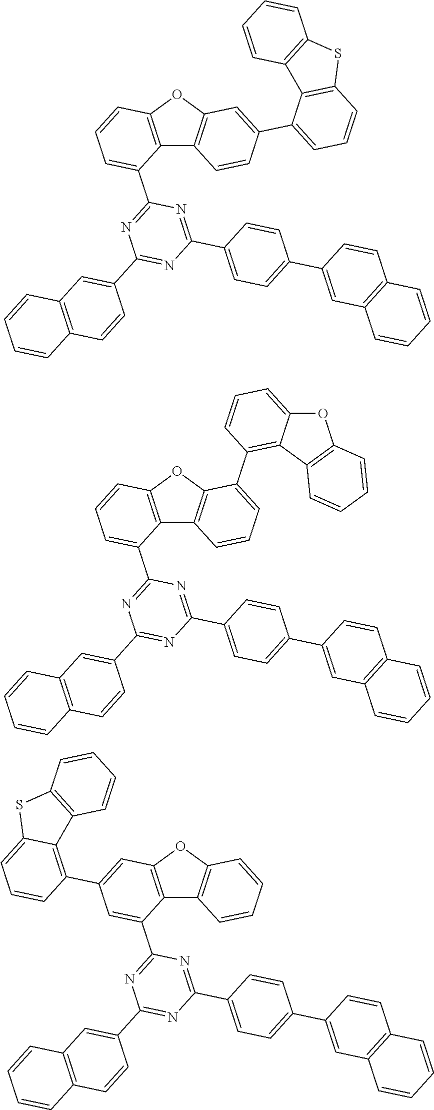

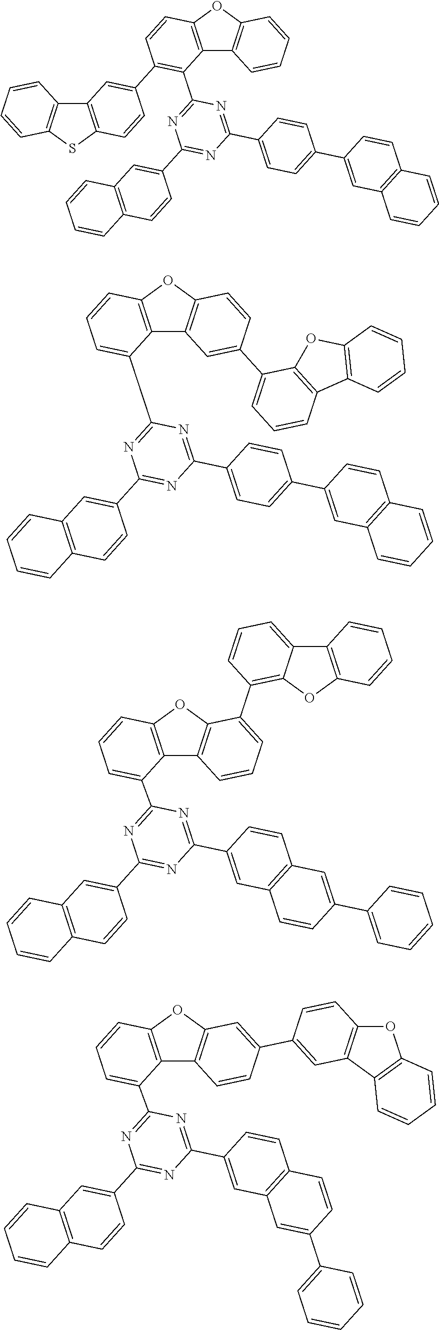 [SMILES: c1ccc2cc(-c3ccc(-c4nc(-c5ccc6ccccc6c5)nc(-c5c(-c6ccc7sc8ccccc8c7c6)ccc6oc7ccccc7c56)n4)cc3)ccc2c1 ; c1ccc2cc(-c3ccc(-c4nc(-c5ccc6ccccc6c5)nc(-c5cccc6oc7ccc(-c8cccc9c8oc8ccccc89)cc7c56)n4)cc3)ccc2c1 ; c1ccc(-c2ccc3cc(-c4nc(-c5ccc6ccccc6c5)nc(-c5cccc6oc7c(-c8cccc9c8oc8ccccc89)cccc7c56)n4)ccc3c2)cc1 ; c1ccc(-c2ccc3ccc(-c4nc(-c5ccc6ccccc6c5)nc(-c5cccc6oc7cc(-c8ccc9oc%10ccccc%10c9c8)ccc7c56)n4)cc3c2)cc1]

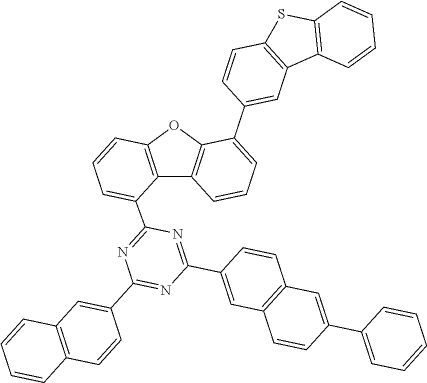 [SMILES: c1ccc(-c2ccc3cc(-c4nc(-c5ccc6ccccc6c5)nc(-c5cccc6oc7c(-c8ccc9sc%10ccccc%10c9c8)cccc7c56)n4)ccc3c2)cc1]

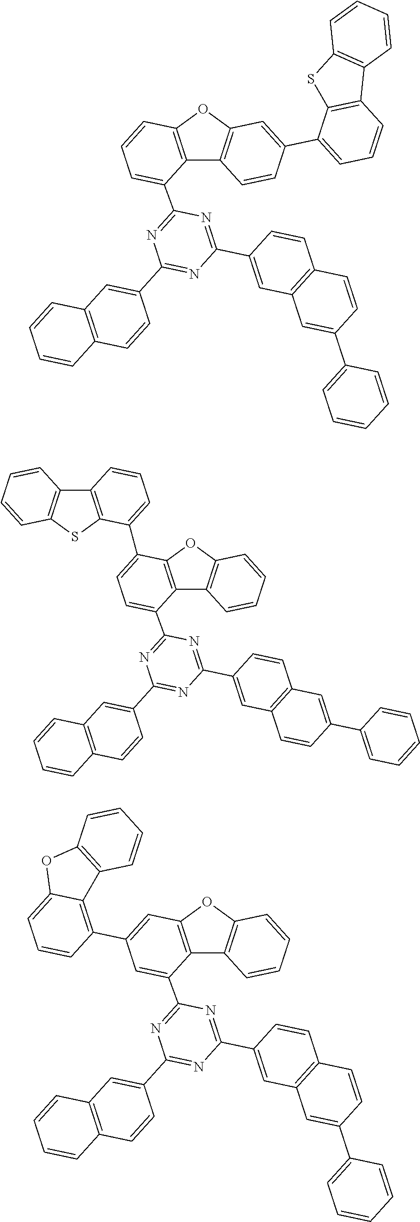

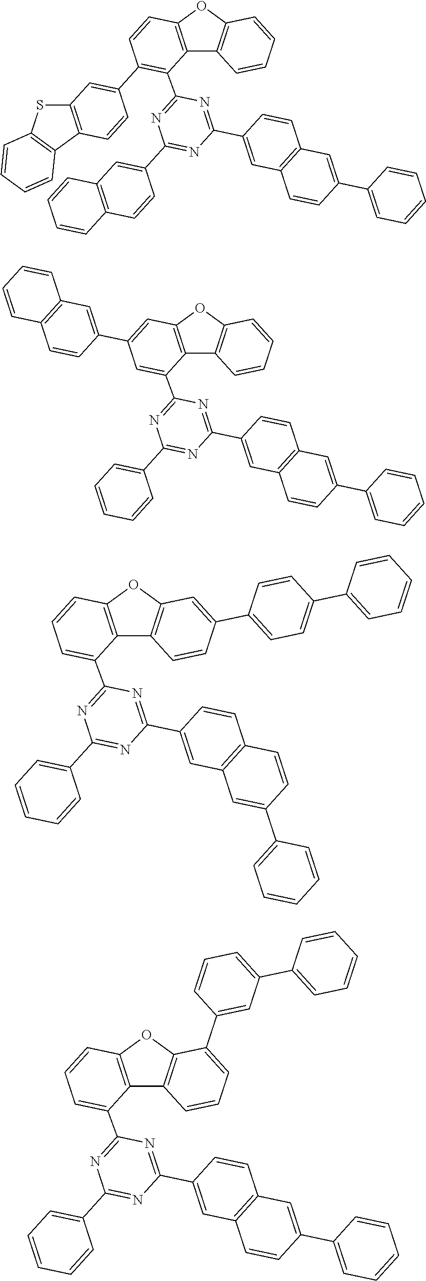

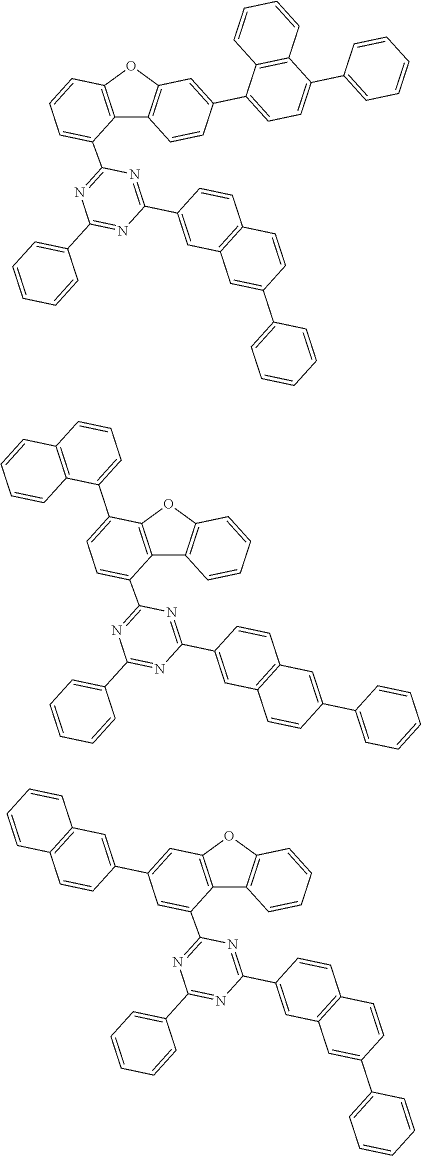 [SMILES: c1ccc(-c2ccc3ccc(-c4nc(-c5ccccc5)nc(-c5cccc6oc7cc(-c8ccc(-c9ccccc9)c9ccccc89)ccc7c56)n4)cc3c2)cc1 ; c1ccc(-c2ccc3cc(-c4nc(-c5ccccc5)nc(-c5ccc(-c6cccc7ccccc67)c6oc7ccccc7c56)n4)ccc3c2)cc1 ; c1ccc(-c2ccc3ccc(-c4nc(-c5ccccc5)nc(-c5cc(-c6ccc7ccccc7c6)cc6oc7ccccc7c56)n4)cc3c2)cc1]

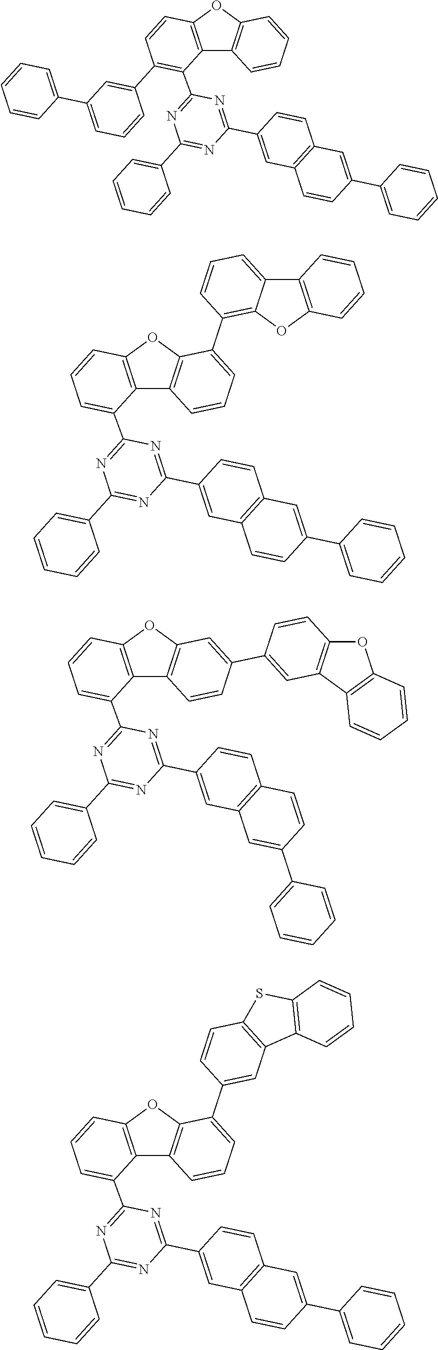

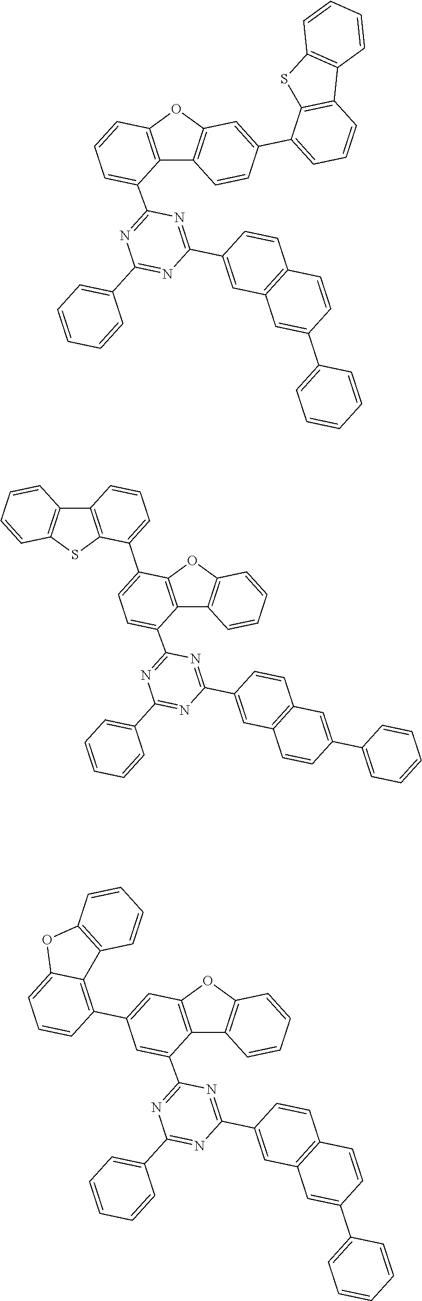 [SMILES: c1ccc(-c2ccc3ccc(-c4nc(-c5ccccc5)nc(-c5cccc6oc7cc(-c8cccc9c8sc8ccccc89)ccc7c56)n4)cc3c2)cc1 ; c1ccc(-c2ccc3cc(-c4nc(-c5ccccc5)nc(-c5ccc(-c6cccc7c6sc6ccccc67)c6oc7ccccc7c56)n4)ccc3c2)cc1 ; c1ccc(-c2ccc3ccc(-c4nc(-c5ccccc5)nc(-c5cc(-c6cccc7oc8ccccc8c67)cc6oc7ccccc7c56)n4)cc3c2)cc1]

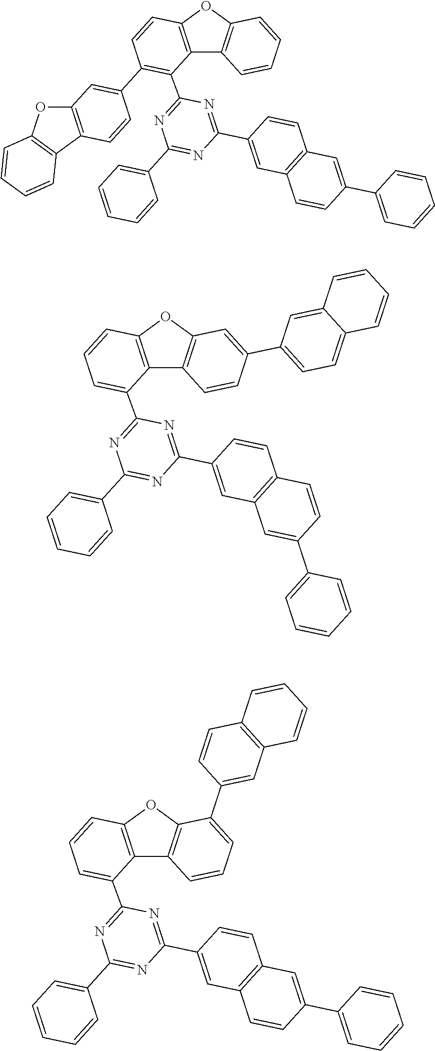 [SMILES: c1ccc(-c2ccc3cc(-c4nc(-c5ccccc5)nc(-c5c(-c6ccc7c(c6)oc6ccccc67)ccc6oc7ccccc7c56)n4)ccc3c2)cc1 ; c1ccc(-c2ccc3ccc(-c4nc(-c5ccccc5)nc(-c5cccc6oc7cc(-c8ccc9ccccc9c8)ccc7c56)n4)cc3c2)cc1 ; c1ccc(-c2ccc3cc(-c4nc(-c5ccccc5)nc(-c5cccc6oc7c(-c8ccc9ccccc9c8)cccc7c56)n4)ccc3c2)cc1]

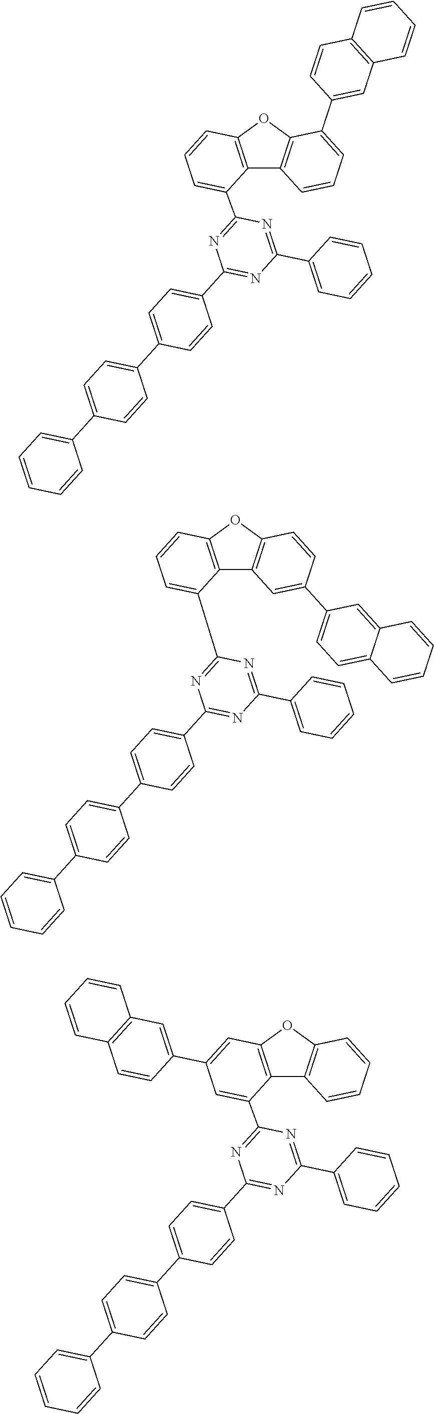 [SMILES: c1ccc(-c2ccc(-c3ccc(-c4nc(-c5ccccc5)nc(-c5cccc6oc7c(-c8ccc9ccccc9c8)cccc7c56)n4)cc3)cc2)cc1 ; c1ccc(-c2ccc(-c3ccc(-c4nc(-c5ccccc5)nc(-c5cccc6oc7ccc(-c8ccc9ccccc9c8)cc7c56)n4)cc3)cc2)cc1 ; c1ccc(-c2ccc(-c3ccc(-c4nc(-c5ccccc5)nc(-c5cc(-c6ccc7ccccc7c6)cc6oc7ccccc7c56)n4)cc3)cc2)cc1]

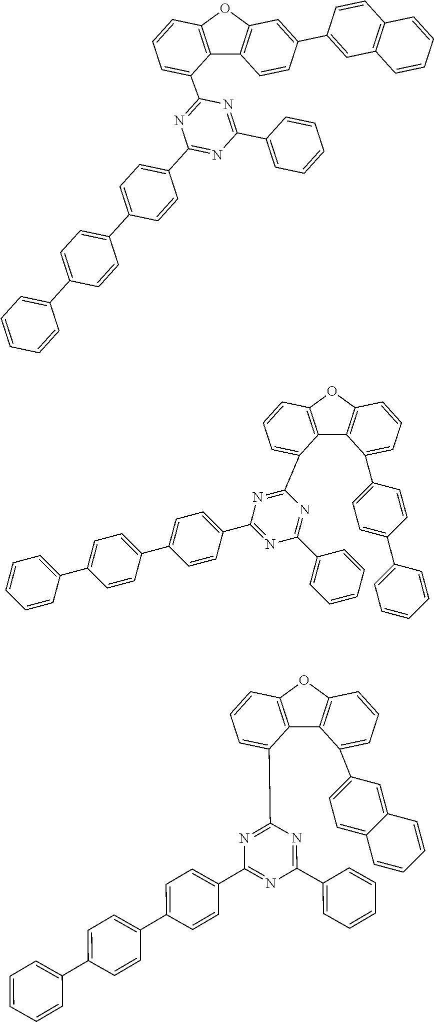 [SMILES: c1ccc(-c2ccc(-c3ccc(-c4nc(-c5ccccc5)nc(-c5cccc6oc7cc(-c8ccc9ccccc9c8)ccc7c56)n4)cc3)cc2)cc1 ; c1ccc(-c2ccc(-c3ccc(-c4nc(-c5ccccc5)nc(-c5cccc6oc7cccc(-c8ccc(-c9ccccc9)cc8)c7c56)n4)cc3)cc2)cc1 ; c1ccc(-c2ccc(-c3ccc(-c4nc(-c5ccccc5)nc(-c5cccc6oc7cccc(-c8ccc9ccccc9c8)c7c56)n4)cc3)cc2)cc1]

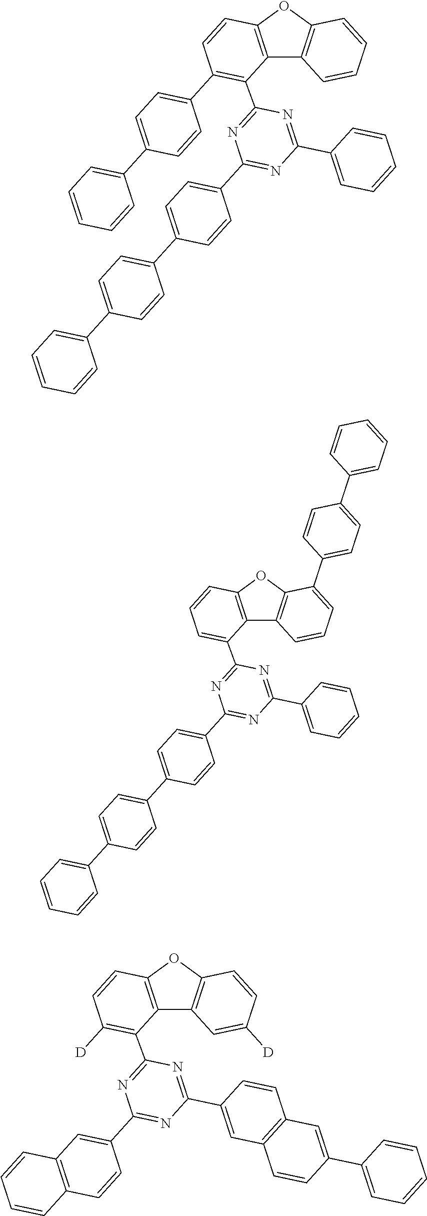 [SMILES: c1ccc(-c2ccc(-c3ccc(-c4nc(-c5ccccc5)nc(-c5c(-c6ccc(-c7ccccc7)cc6)ccc6oc7ccccc7c56)n4)cc3)cc2)cc1 ; c1ccc(-c2ccc(-c3ccc(-c4nc(-c5ccccc5)nc(-c5cccc6oc7c(-c8ccc(-c9ccccc9)cc8)cccc7c56)n4)cc3)cc2)cc1 ; [2H]c1ccc2oc3ccc([2H])c(-c4nc(-c5ccc6ccccc6c5)nc(-c5ccc6cc(-c7ccccc7)ccc6c5)n4)c3c2c1]

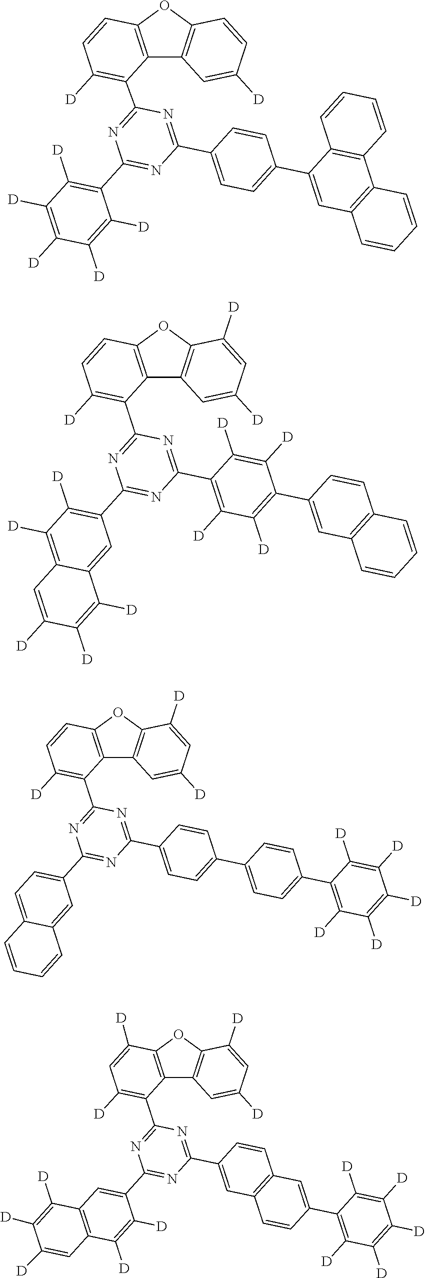

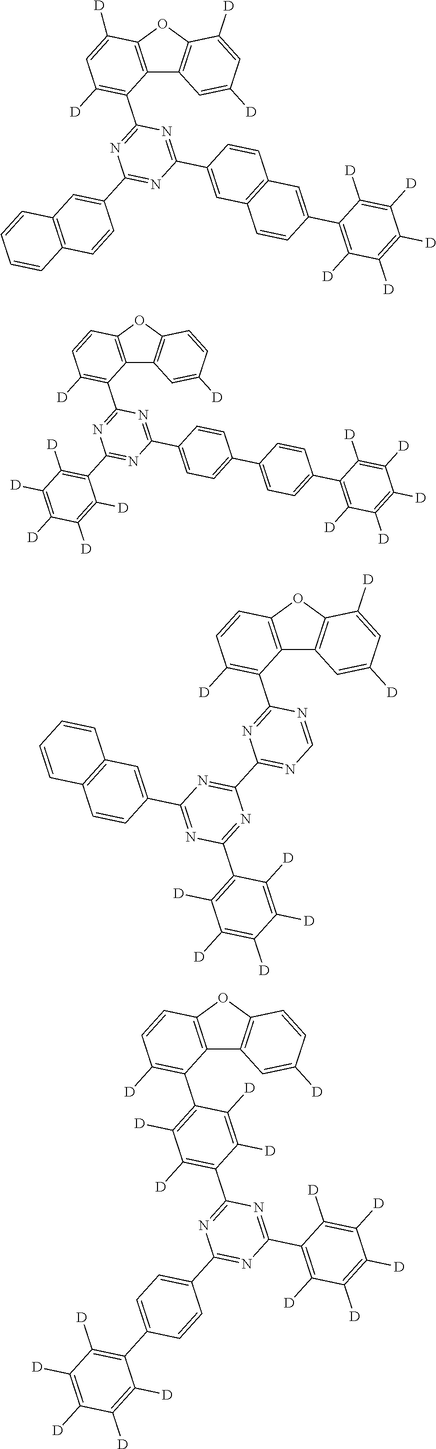

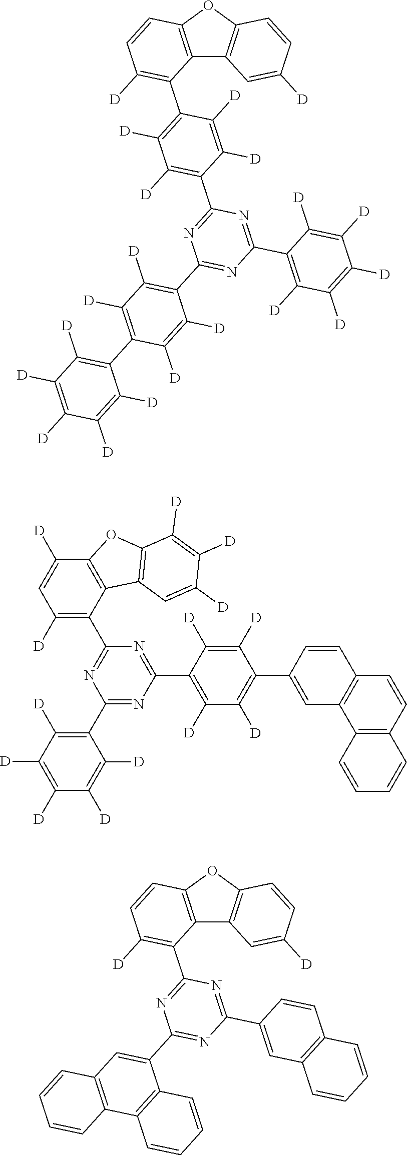 [SMILES: [2H]c1ccc2oc3ccc([2H])c(-c4c([2H])c([2H])c(-c5nc(-c6c([2H])c([2H])c([2H])c([2H])c6[2H])nc(-c6c([2H])c([2H])c(-c7c([2H])c([2H])c([2H])c([2H])c7[2H])c([2H])c6[2H])n5)c([2H])c4[2H])c3c2c1 ; [2H]c1cc2c(oc3c([2H])cc([2H])c(-c4nc(-c5c([2H])c([2H])c([2H])c([2H])c5[2H])nc(-c5c([2H])c([2H])c(-c6ccc7ccc8ccccc8c7c6)c([2H])c5[2H])n4)c32)c([2H])c1[2H] ; [2H]c1ccc2oc3ccc([2H])c(-c4nc(-c5ccc6ccccc6c5)nc(-c5cc6ccccc6c6ccccc56)n4)c3c2c1]

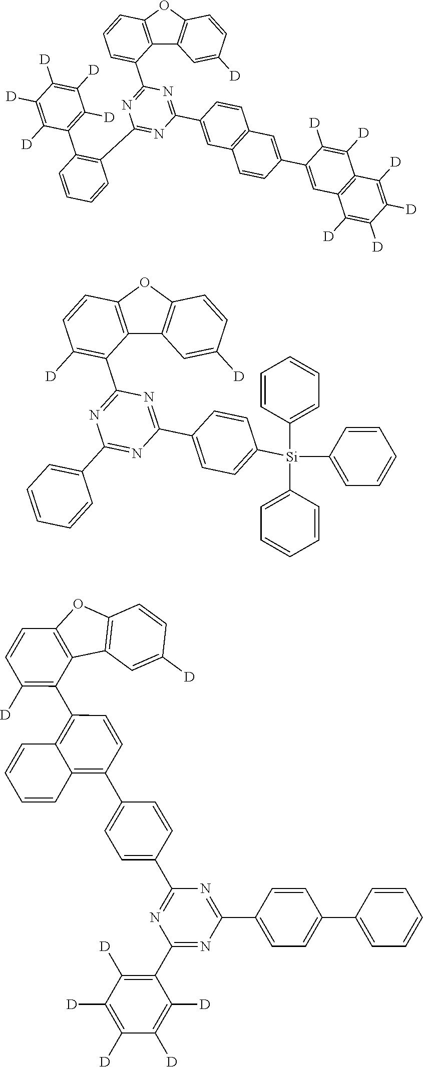 [SMILES: [2H]c1ccc2oc3cccc(-c4nc(-c5ccc6cc(-c7cc8c([2H])c([2H])c([2H])c([2H])c8c([2H])c7[2H])ccc6c5)nc(-c5ccccc5-c5c([2H])c([2H])c([2H])c([2H])c5[2H])n4)c3c2c1 ; [2H]c1ccc2oc3ccc([2H])c(-c4nc(-c5ccccc5)nc(-c5ccc([Si](c6ccccc6)(c6ccccc6)c6ccccc6)cc5)n4)c3c2c1 ; [2H]c1ccc2oc3ccc([2H])c(-c4ccc(-c5ccc(-c6nc(-c7ccc(-c8ccccc8)cc7)nc(-c7c([2H])c([2H])c([2H])c([2H])c7[2H])n6)cc5)c5ccccc45)c3c2c1]

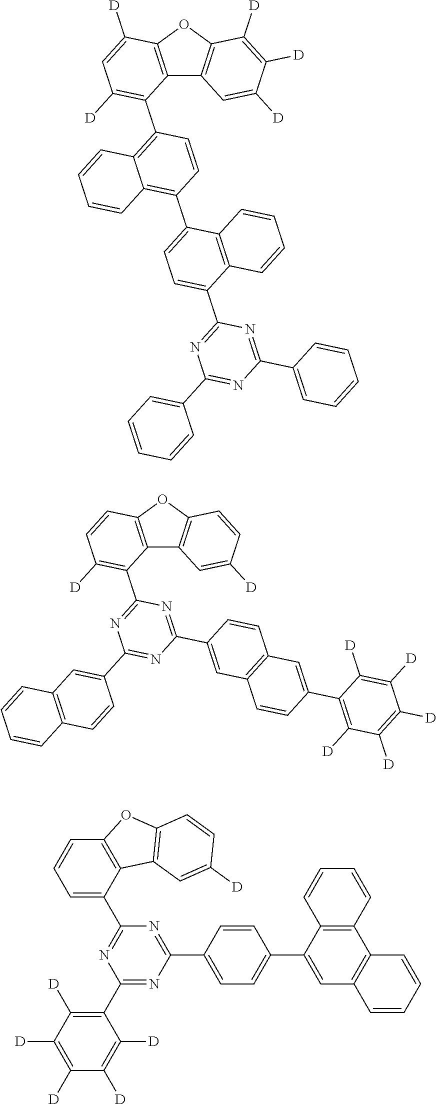

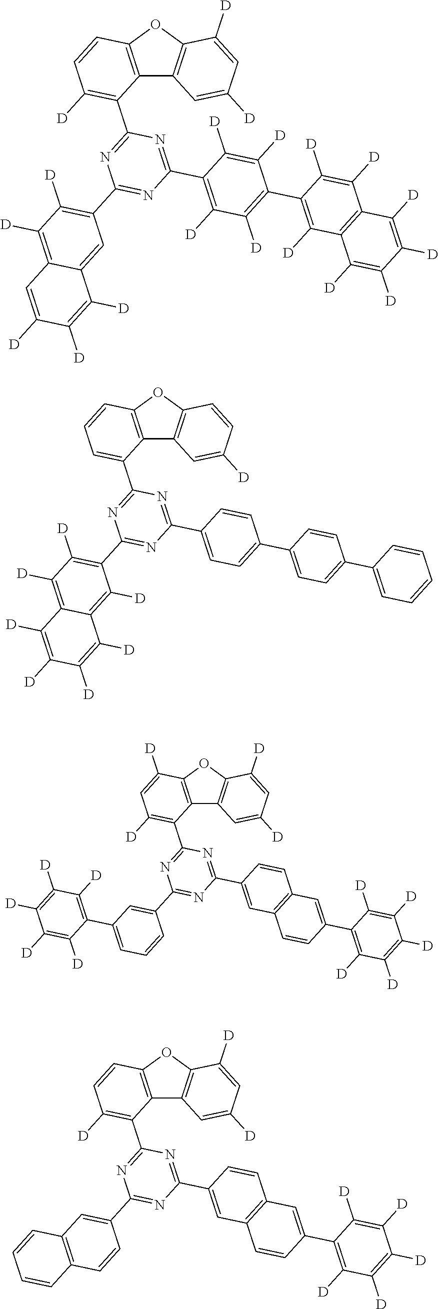

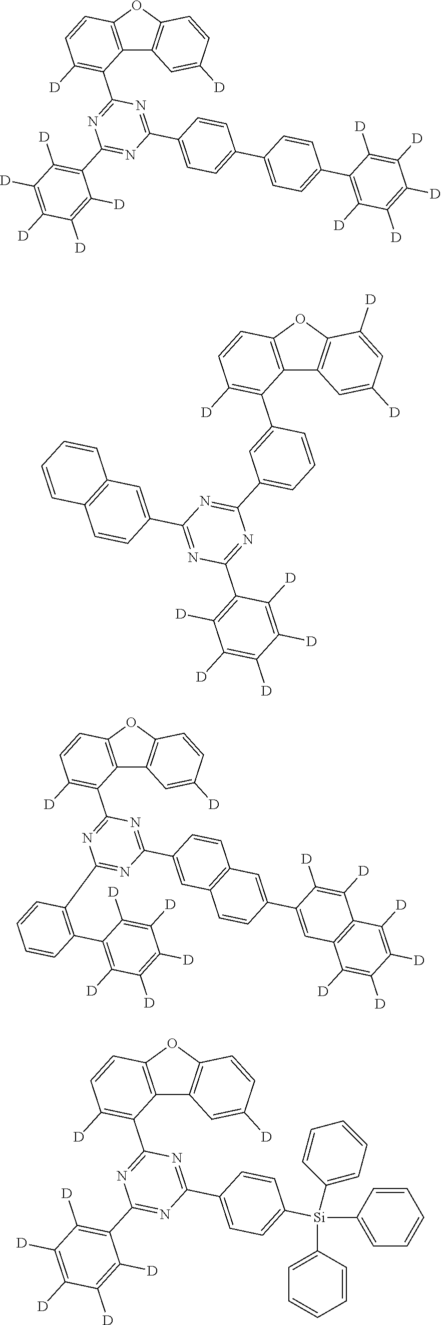

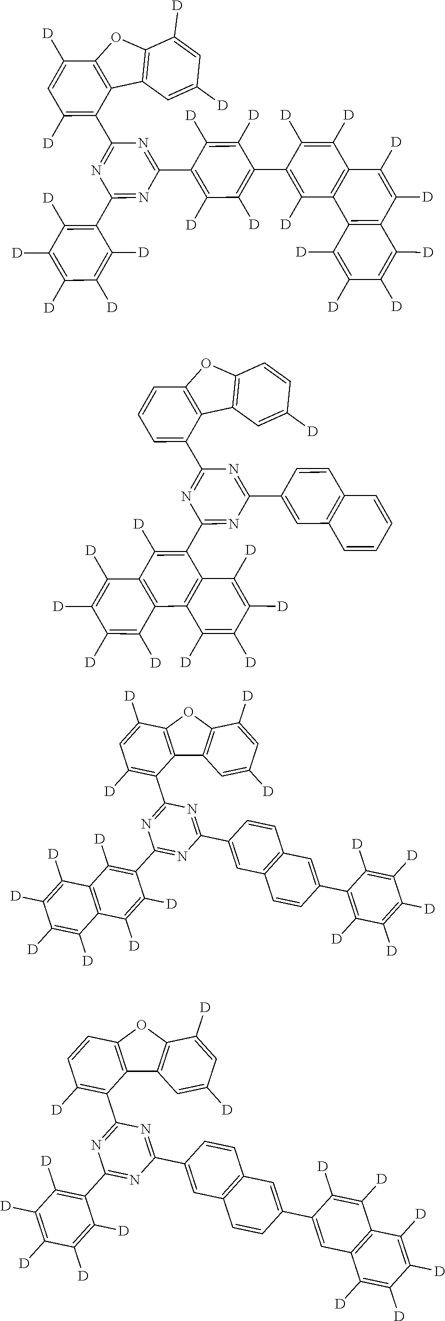

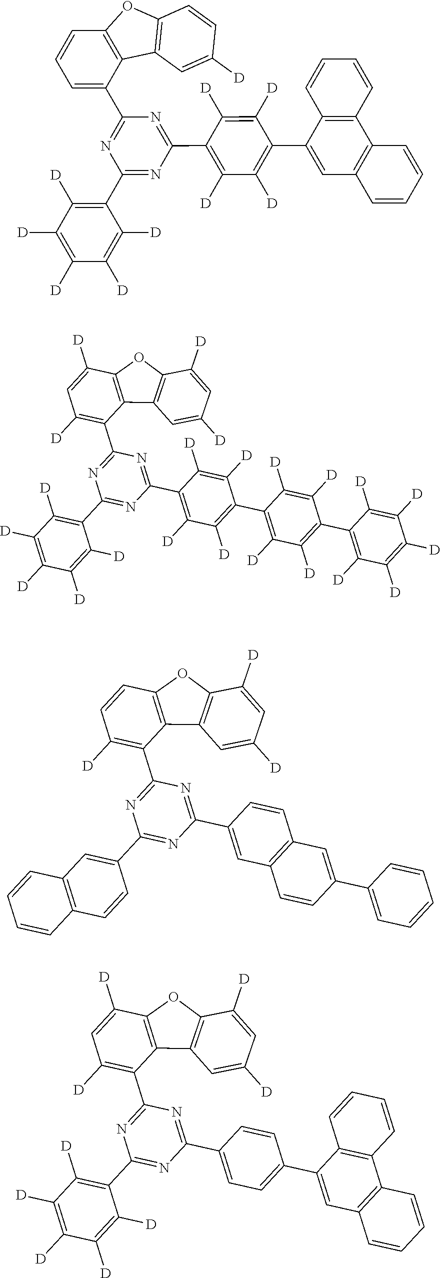

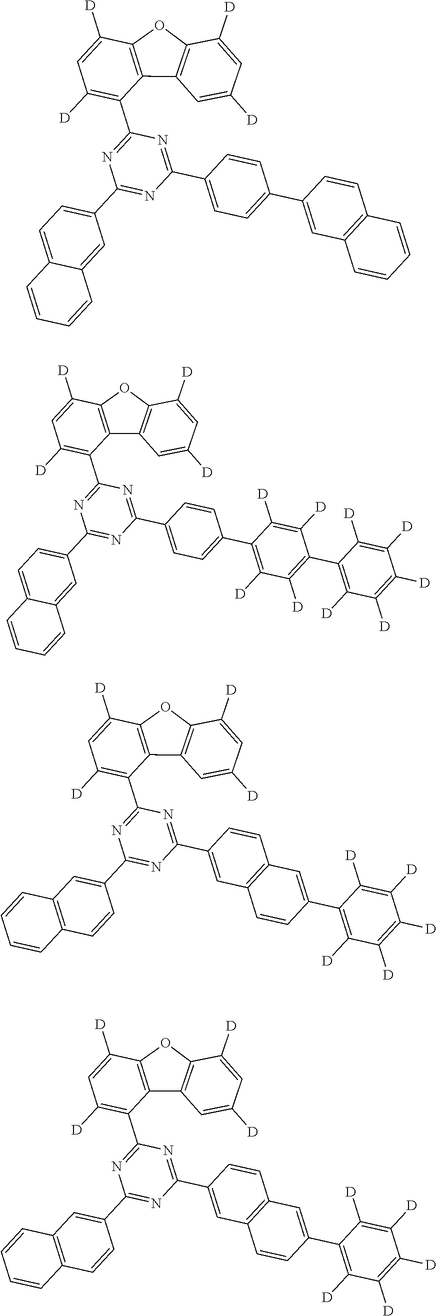

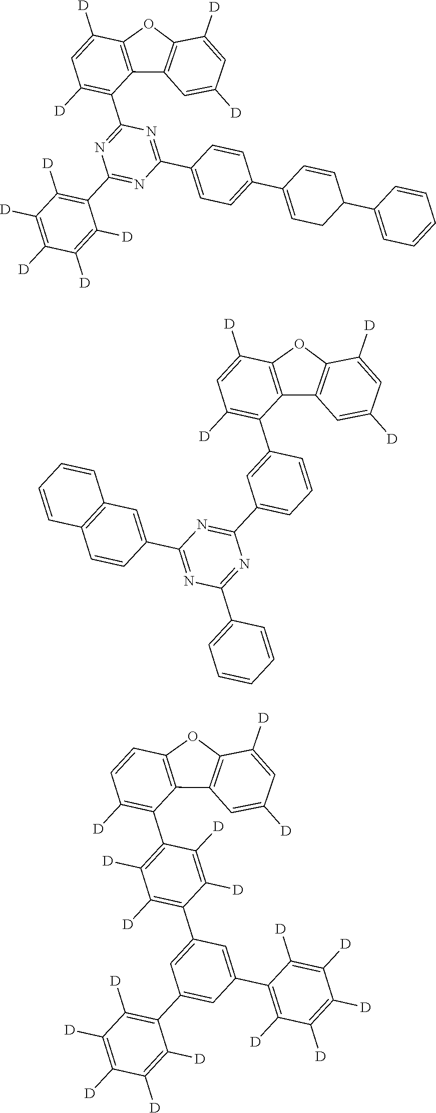

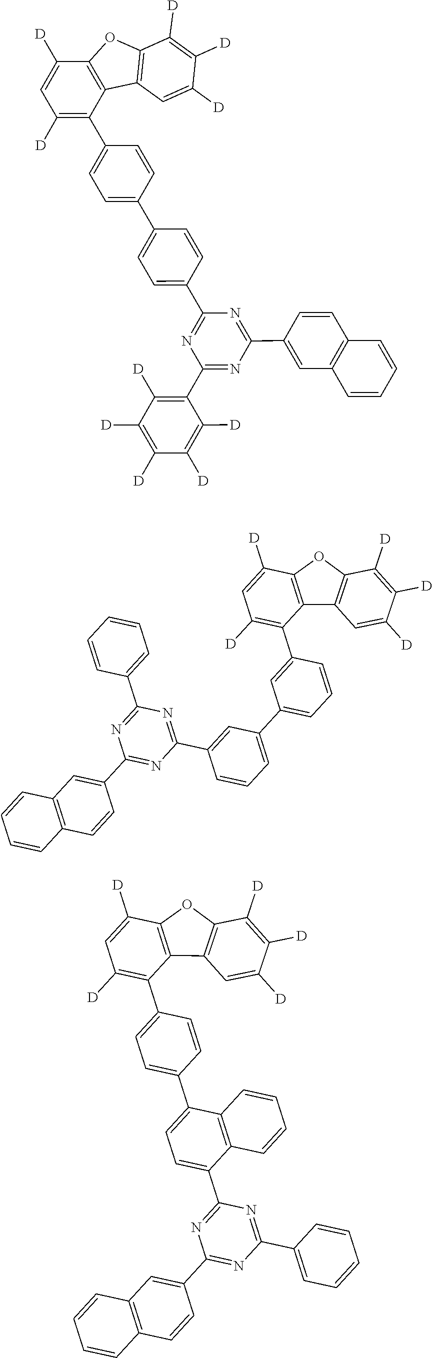 [SMILES: [2H]c1cc2c(oc3c([2H])cc([2H])c(-c4ccc(-c5ccc(-c6nc(-c7ccc8ccccc8c7)nc(-c7c([2H])c([2H])c([2H])c([2H])c7[2H])n6)cc5)cc4)c32)c([2H])c1[2H] ; [2H]c1cc2c(oc3c([2H])cc([2H])c(-c4cccc(-c5cccc(-c6nc(-c7ccccc7)nc(-c7ccc8ccccc8c7)n6)c5)c4)c32)c([2H])c1[2H] ; [2H]c1cc2c(oc3c([2H])cc([2H])c(-c4ccc(-c5ccc(-c6nc(-c7ccccc7)nc(-c7ccc8ccccc8c7)n6)c6ccccc56)cc4)c32)c([2H])c1[2H]]

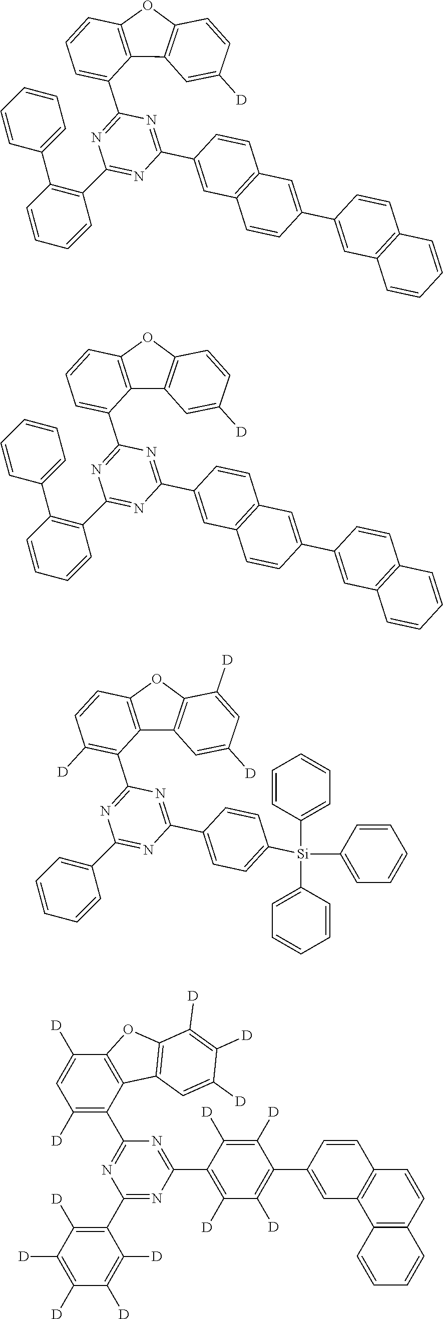 [SMILES: [2H]c1ccc2oc3cccc(-c4nc(-c5ccc6cc(-c7ccc8ccccc8c7)ccc6c5)nc(-c5ccccc5-c5ccccc5)n4)c3c2c1 ; [2H]c1ccc2oc3cccc(-c4nc(-c5ccc6cc(-c7ccc8ccccc8c7)ccc6c5)nc(-c5ccccc5-c5ccccc5)n4)c3c2c1 ; [2H]c1cc([2H])c2oc3ccc([2H])c(-c4nc(-c5ccccc5)nc(-c5ccc([Si](c6ccccc6)(c6ccccc6)c6ccccc6)cc5)n4)c3c2c1 ; [2H]c1cc2c(oc3c([2H])cc([2H])c(-c4nc(-c5c([2H])c([2H])c([2H])c([2H])c5[2H])nc(-c5c([2H])c([2H])c(-c6ccc7ccc8ccccc8c7c6)c([2H])c5[2H])n4)c32)c([2H])c1[2H]]

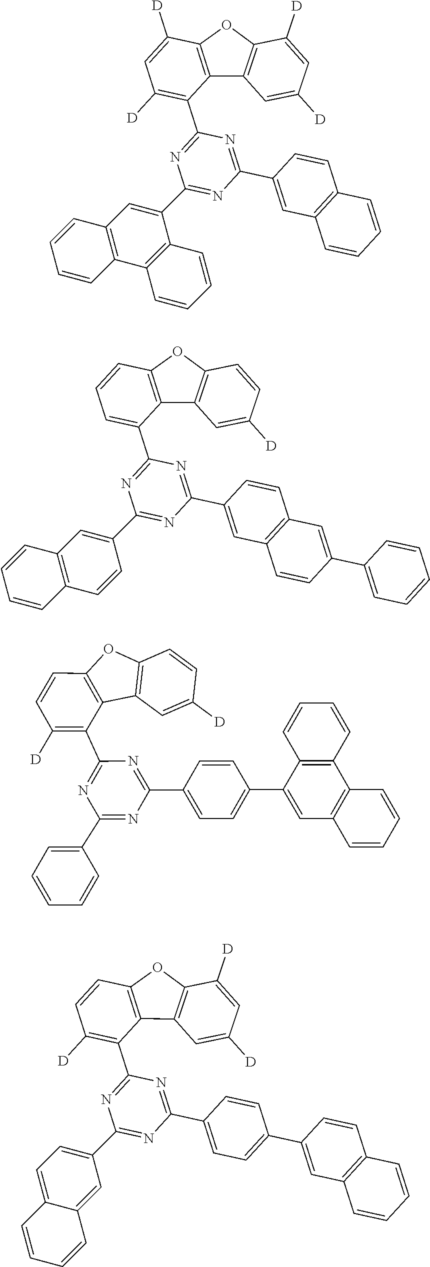

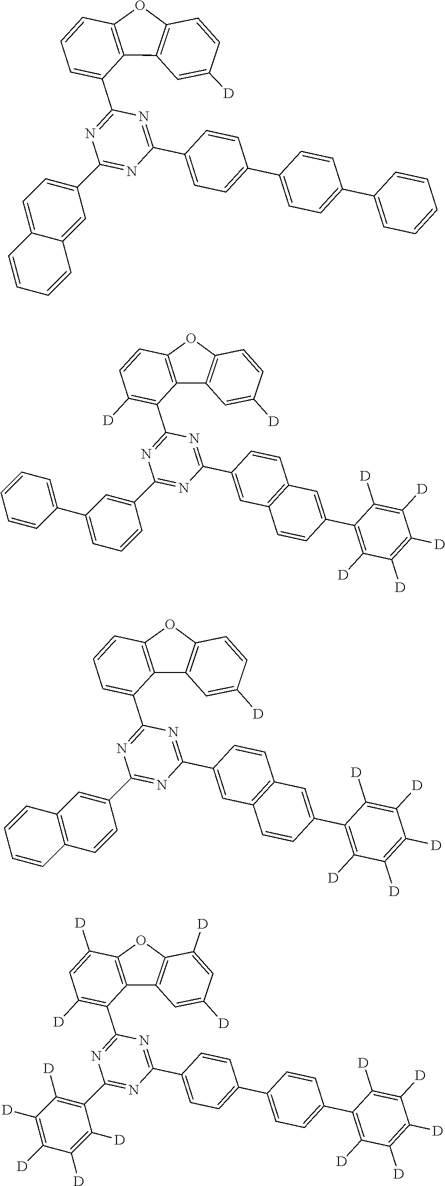 [SMILES: [2H]c1ccc2oc3cccc(-c4nc(-c5ccc(-c6ccc(-c7ccccc7)cc6)cc5)nc(-c5ccc6ccccc6c5)n4)c3c2c1 ; [2H]c1ccc2oc3ccc([2H])c(-c4nc(-c5cccc(-c6ccccc6)c5)nc(-c5ccc6cc(-c7c([2H])c([2H])c([2H])c([2H])c7[2H])ccc6c5)n4)c3c2c1 ; [2H]c1ccc2oc3cccc(-c4nc(-c5ccc6ccccc6c5)nc(-c5ccc6cc(-c7c([2H])c([2H])c([2H])c([2H])c7[2H])ccc6c5)n4)c3c2c1 ; [2H]c1cc([2H])c2oc3c([2H])cc([2H])c(-c4nc(-c5ccc(-c6ccc(-c7c([2H])c([2H])c([2H])c([2H])c7[2H])cc6)cc5)nc(-c5c([2H])c([2H])c([2H])c([2H])c5[2H])n4)c3c2c1]

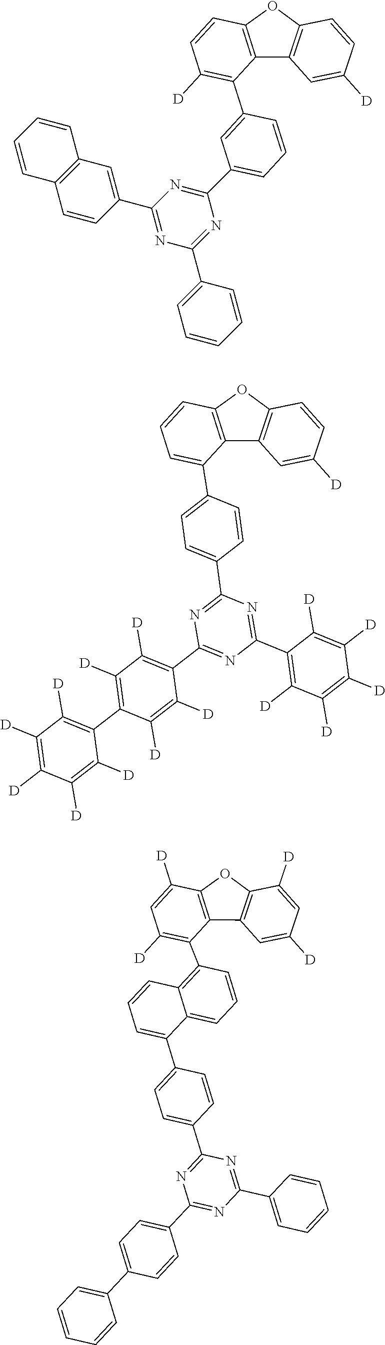 [SMILES: [2H]c1ccc2oc3ccc([2H])c(-c4cccc(-c5nc(-c6ccccc6)nc(-c6ccc7ccccc7c6)n5)c4)c3c2c1 ; [2H]c1ccc2oc3cccc(-c4ccc(-c5nc(-c6c([2H])c([2H])c([2H])c([2H])c6[2H])nc(-c6c([2H])c([2H])c(-c7c([2H])c([2H])c([2H])c([2H])c7[2H])c([2H])c6[2H])n5)cc4)c3c2c1 ; [2H]c1cc([2H])c2oc3c([2H])cc([2H])c(-c4cccc5c(-c6ccc(-c7nc(-c8ccccc8)nc(-c8ccc(-c9ccccc9)cc8)n7)cc6)cccc45)c3c2c1]

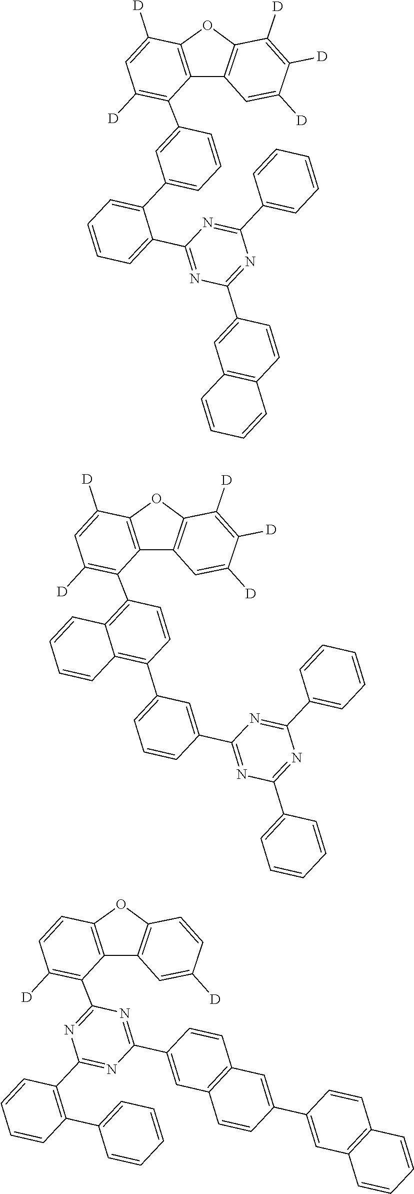

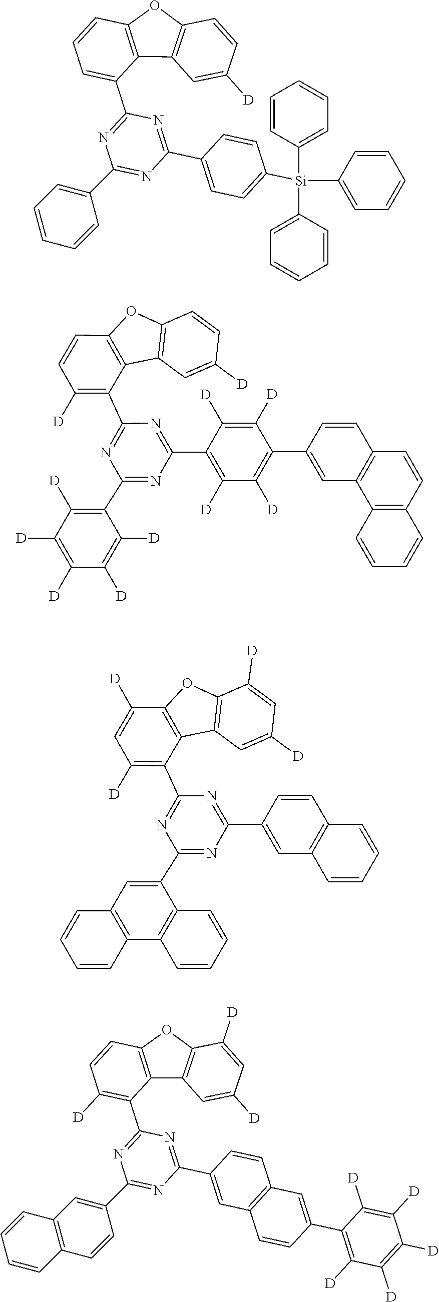 [SMILES: [2H]c1ccc2oc3cccc(-c4nc(-c5ccccc5)nc(-c5ccc([Si](c6ccccc6)(c6ccccc6)c6ccccc6)cc5)n4)c3c2c1 ; [2H]c1ccc2oc3ccc([2H])c(-c4nc(-c5c([2H])c([2H])c([2H])c([2H])c5[2H])nc(-c5c([2H])c([2H])c(-c6ccc7ccc8ccccc8c7c6)c([2H])c5[2H])n4)c3c2c1 ; [2H]c1cc([2H])c2oc3c([2H])cc([2H])c(-c4nc(-c5ccc6ccccc6c5)nc(-c5cc6ccccc6c6ccccc56)n4)c3c2c1 ; [2H]c1cc([2H])c2oc3ccc([2H])c(-c4nc(-c5ccc6ccccc6c5)nc(-c5ccc6cc(-c7c([2H])c([2H])c([2H])c([2H])c7[2H])ccc6c5)n4)c3c2c1]

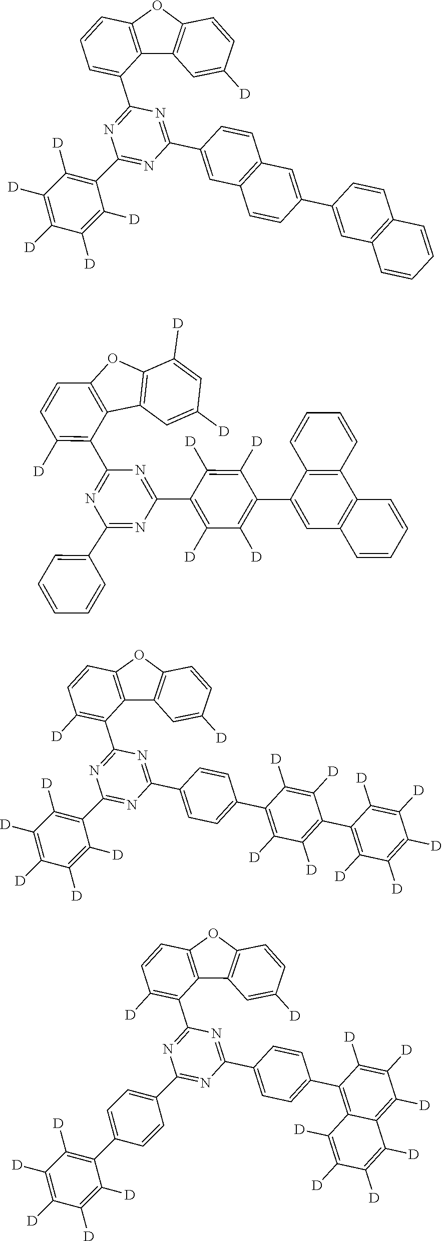

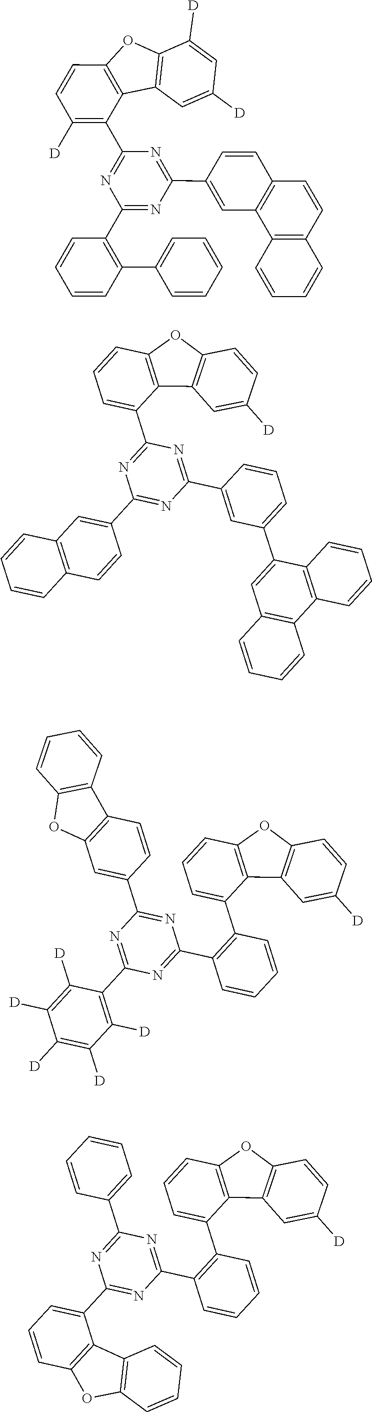

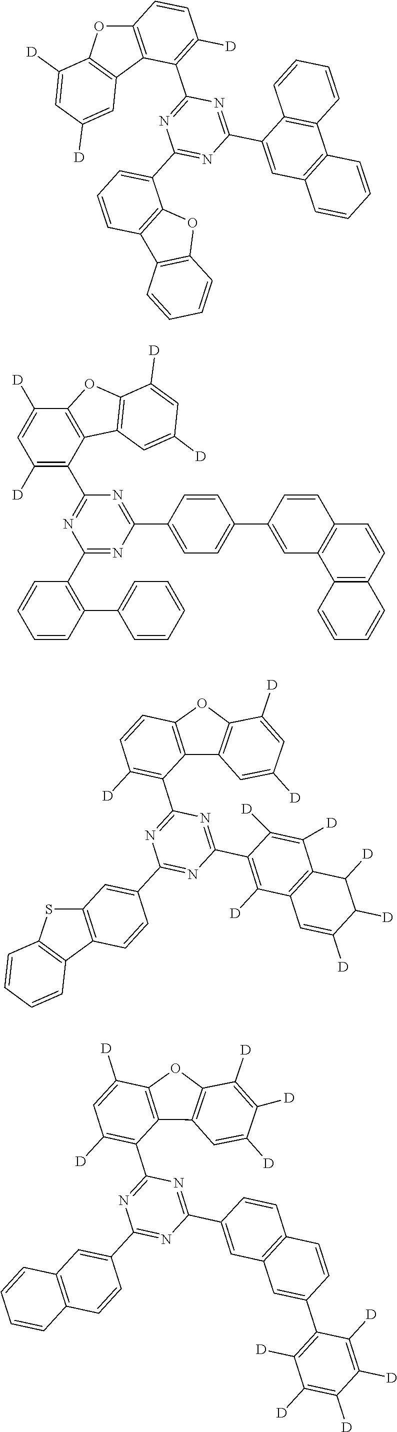

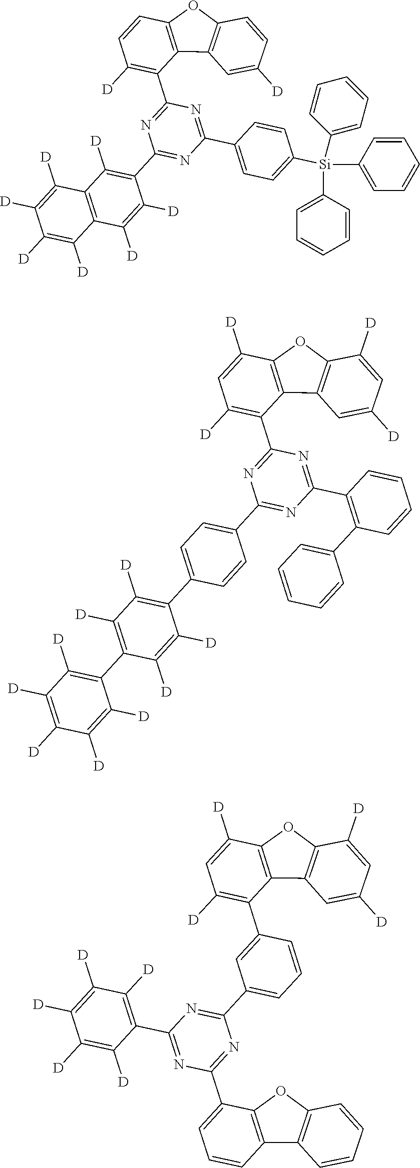 [SMILES: [2H]c1ccc2oc3ccc([2H])c(-c4nc(-c5ccc([Si](c6ccccc6)(c6ccccc6)c6ccccc6)cc5)nc(-c5c([2H])c([2H])c6c([2H])c([2H])c([2H])c([2H])c6c5[2H])n4)c3c2c1 ; [2H]c1cc([2H])c2oc3c([2H])cc([2H])c(-c4nc(-c5ccc(-c6c([2H])c([2H])c(-c7c([2H])c([2H])c([2H])c([2H])c7[2H])c([2H])c6[2H])cc5)nc(-c5ccccc5-c5ccccc5)n4)c3c2c1 ; [2H]c1cc([2H])c2oc3c([2H])cc([2H])c(-c4cccc(-c5nc(-c6c([2H])c([2H])c([2H])c([2H])c6[2H])nc(-c6cccc7c6oc6ccccc67)n5)c4)c3c2c1]

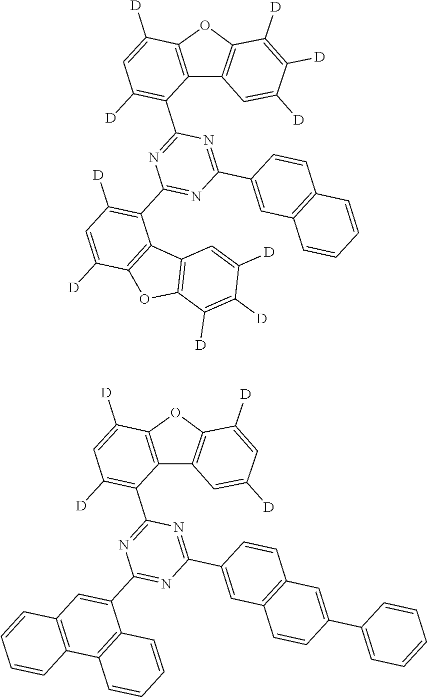 [SMILES: [2H]c1cc2c(oc3c([2H])cc([2H])c(-c4nc(-c5ccc6ccccc6c5)nc(-c5c([2H])cc([2H])c6oc7c([2H])c([2H])c([2H])cc7c56)n4)c32)c([2H])c1[2H] ; [2H]c1cc([2H])c2oc3c([2H])cc([2H])c(-c4nc(-c5ccc6cc(-c7ccccc7)ccc6c5)nc(-c5cc6ccccc6c6ccccc56)n4)c3c2c1]

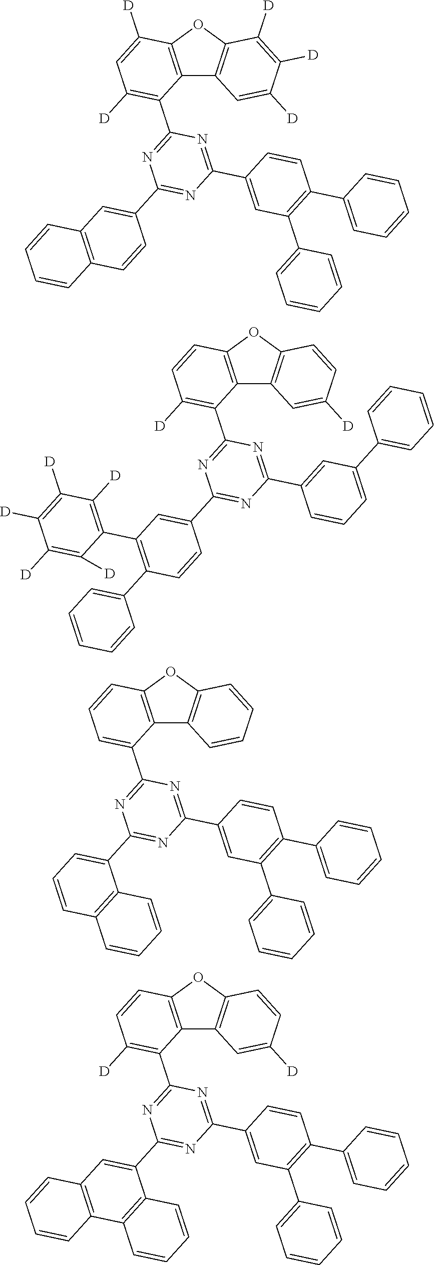

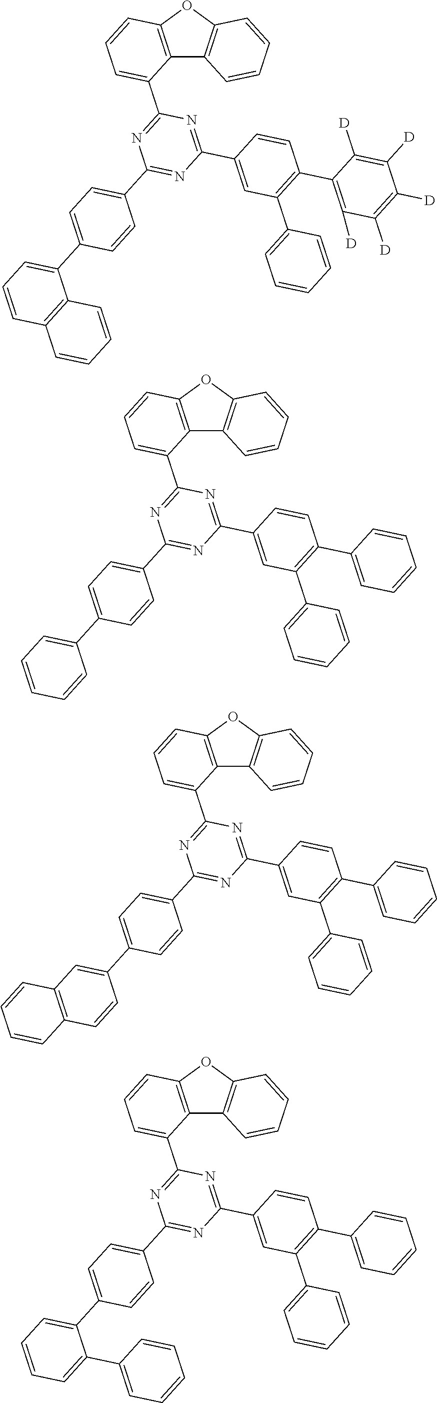 [SMILES: [2H]c1c([2H])c([2H])c(-c2ccc(-c3nc(-c4ccc(-c5cccc6ccccc56)cc4)nc(-c4cccc5oc6ccccc6c45)n3)cc2-c2ccccc2)c([2H])c1[2H] ; c1ccc(-c2ccc(-c3nc(-c4ccc(-c5ccccc5)c(-c5ccccc5)c4)nc(-c4cccc5oc6ccccc6c45)n3)cc2)cc1 ; c1ccc(-c2ccc(-c3nc(-c4ccc(-c5ccc6ccccc6c5)cc4)nc(-c4cccc5oc6ccccc6c45)n3)cc2-c2ccccc2)cc1 ; c1ccc(-c2ccccc2-c2ccc(-c3nc(-c4ccc(-c5ccccc5)c(-c5ccccc5)c4)nc(-c4cccc5oc6ccccc6c45)n3)cc2)cc1]

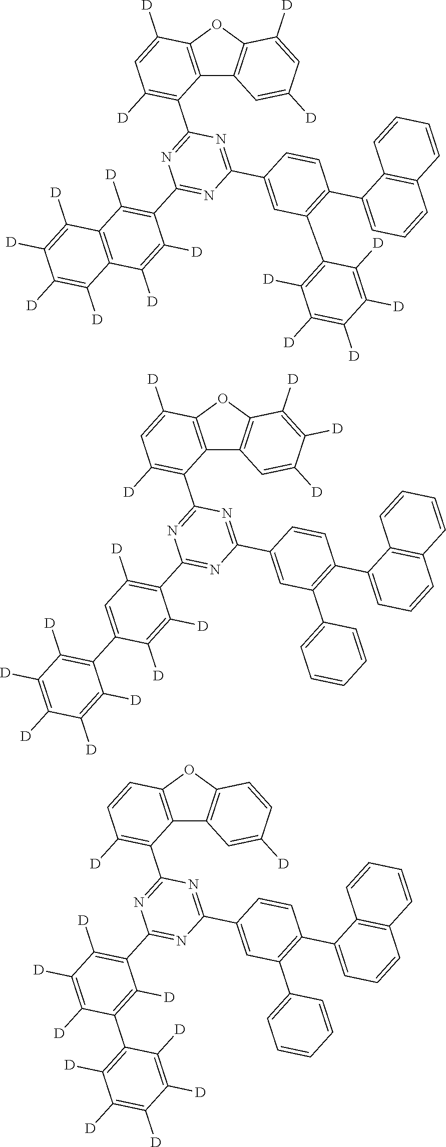

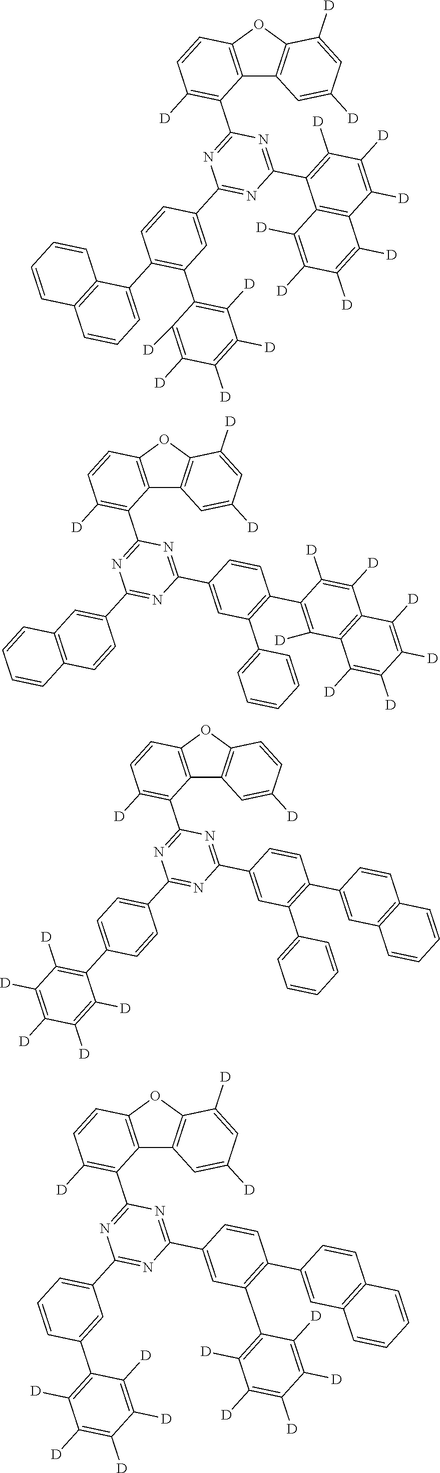

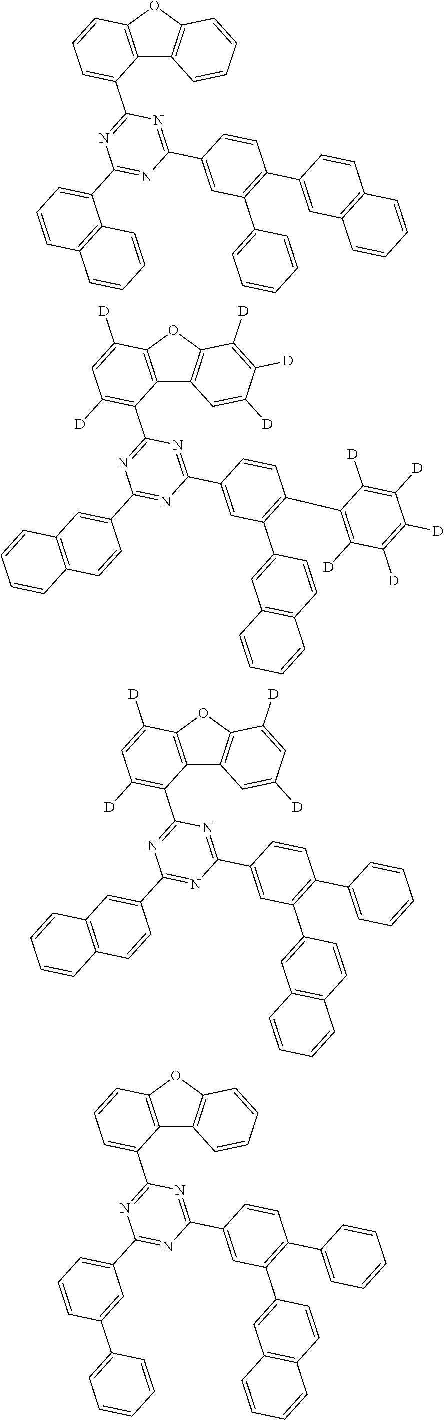

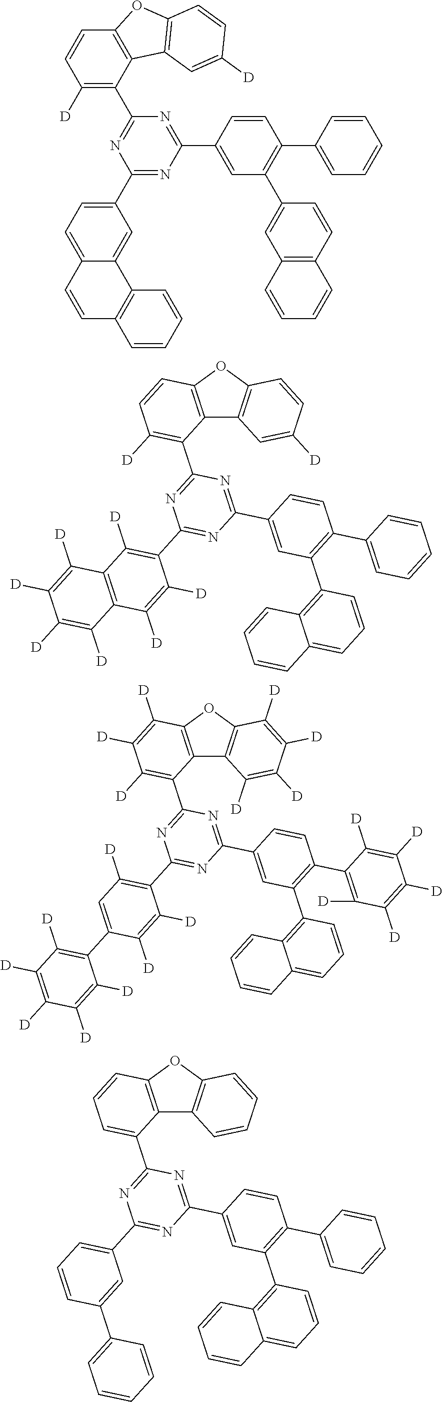

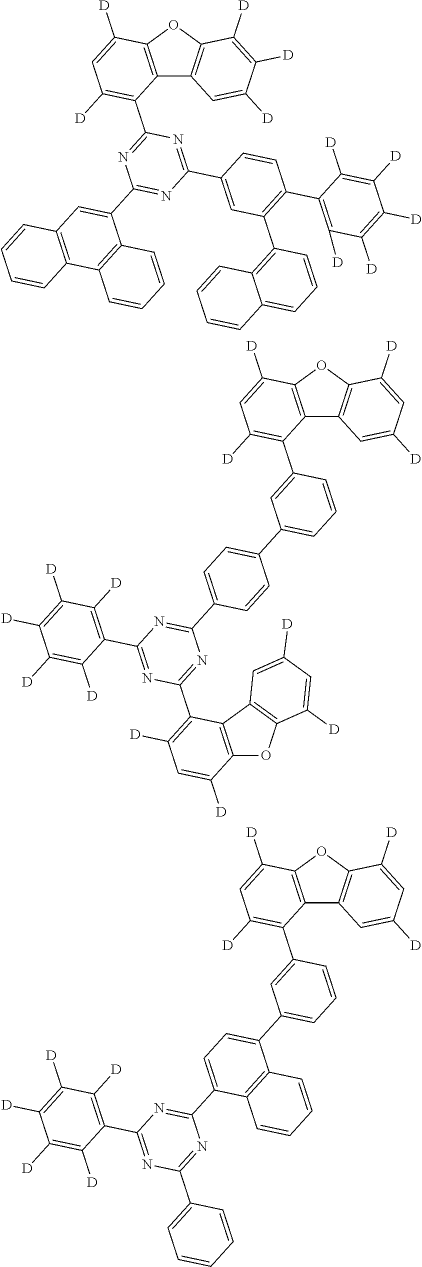

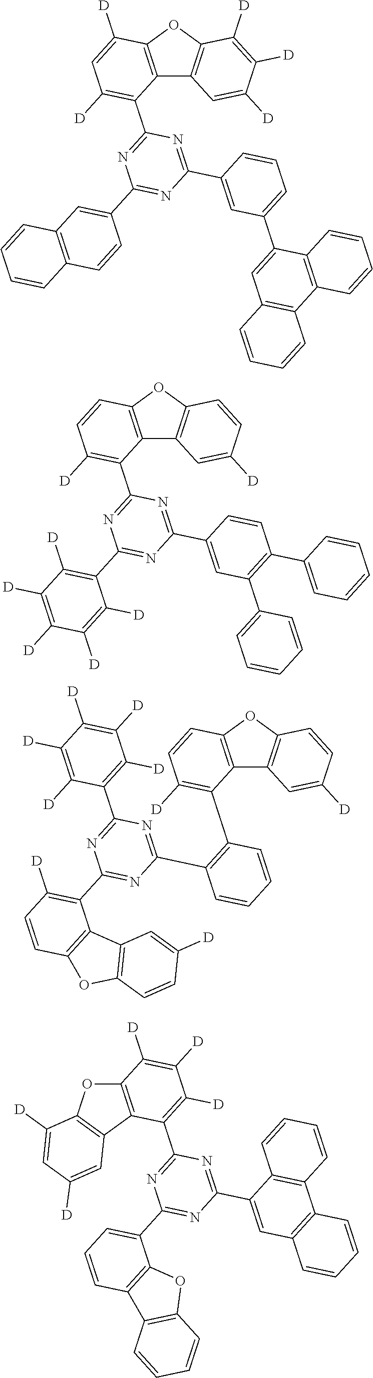

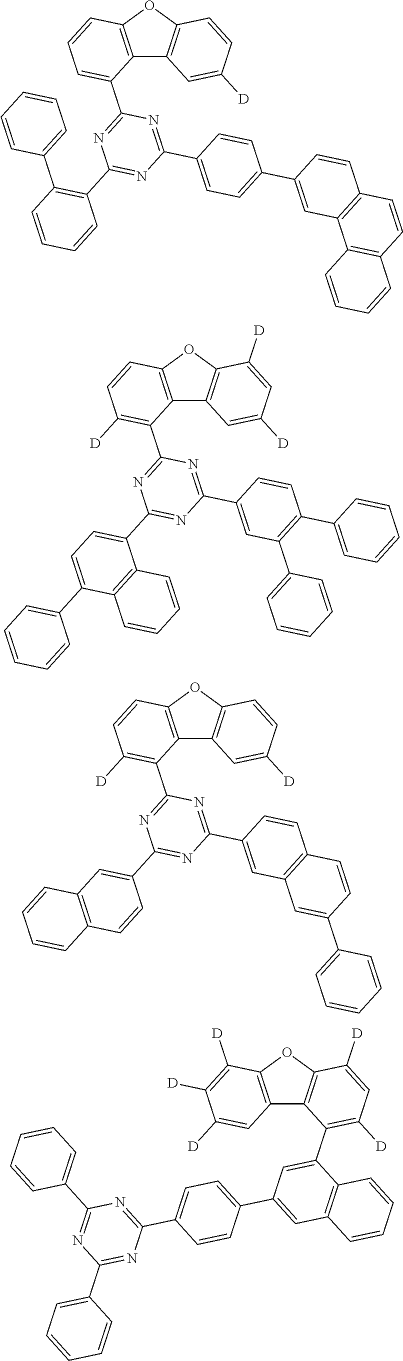

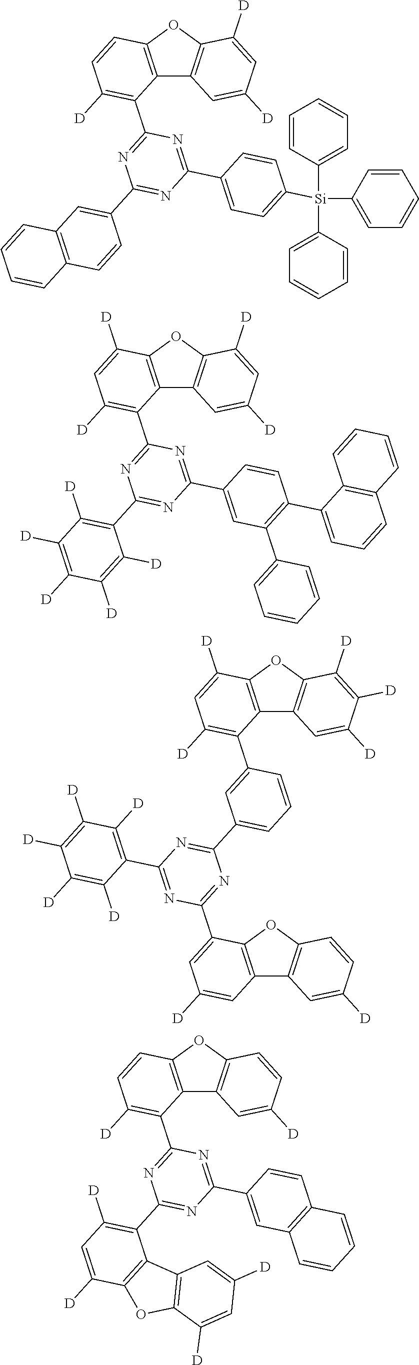

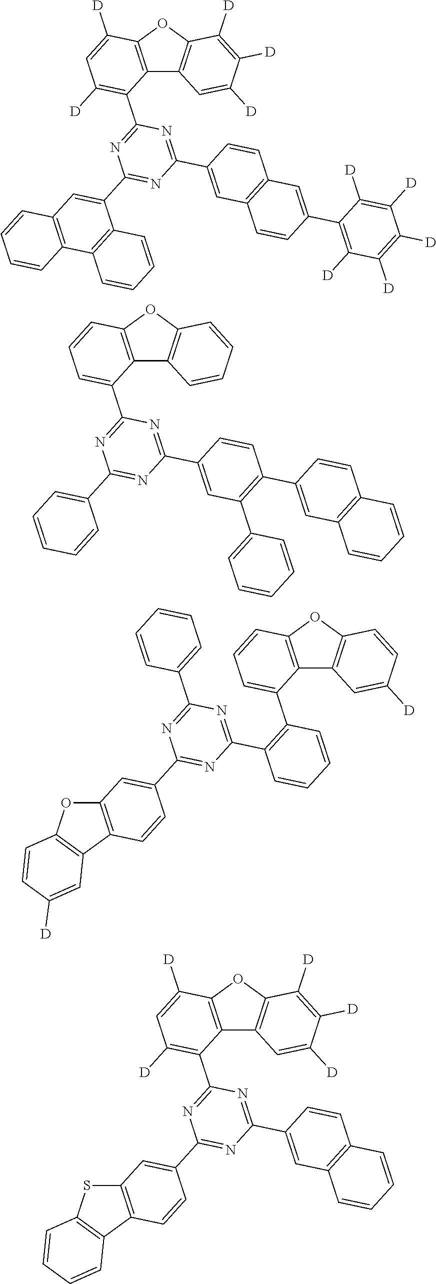

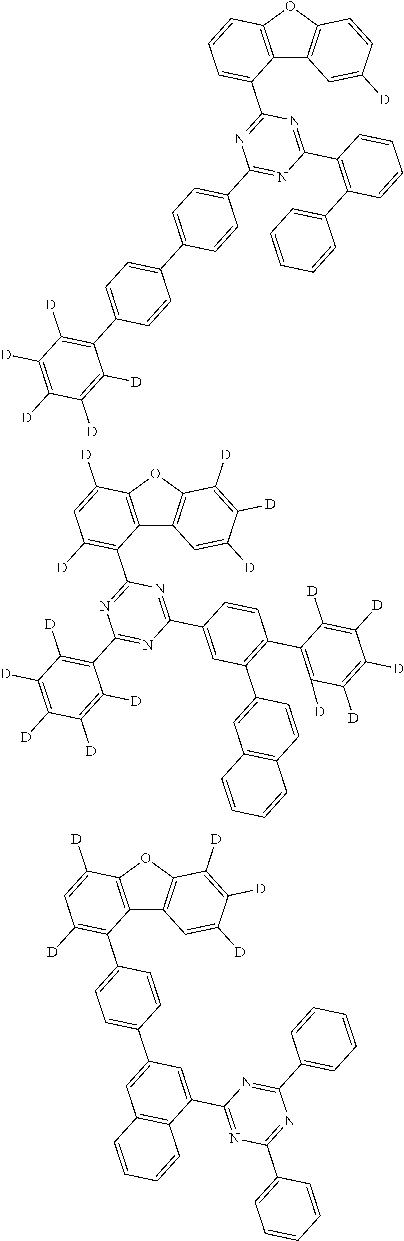

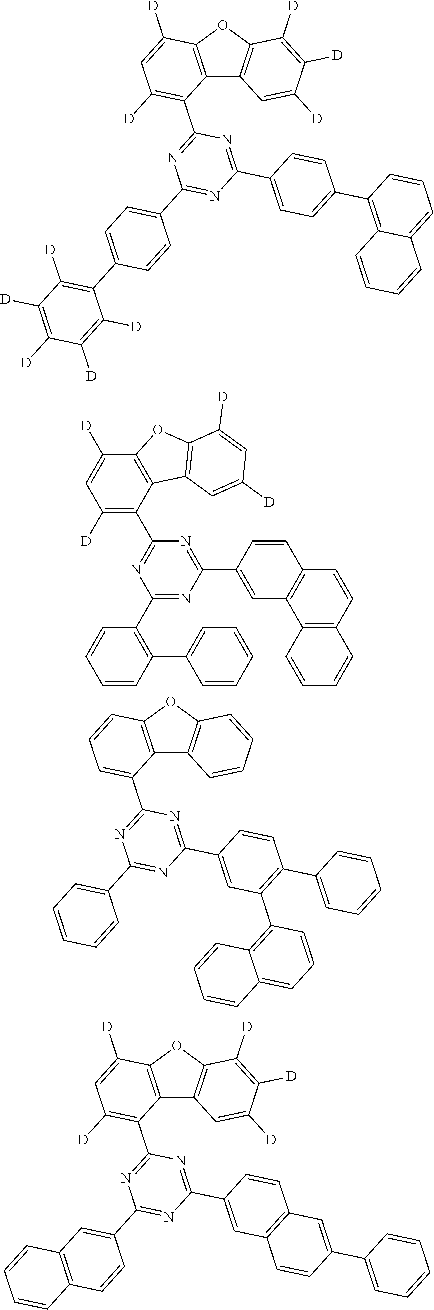

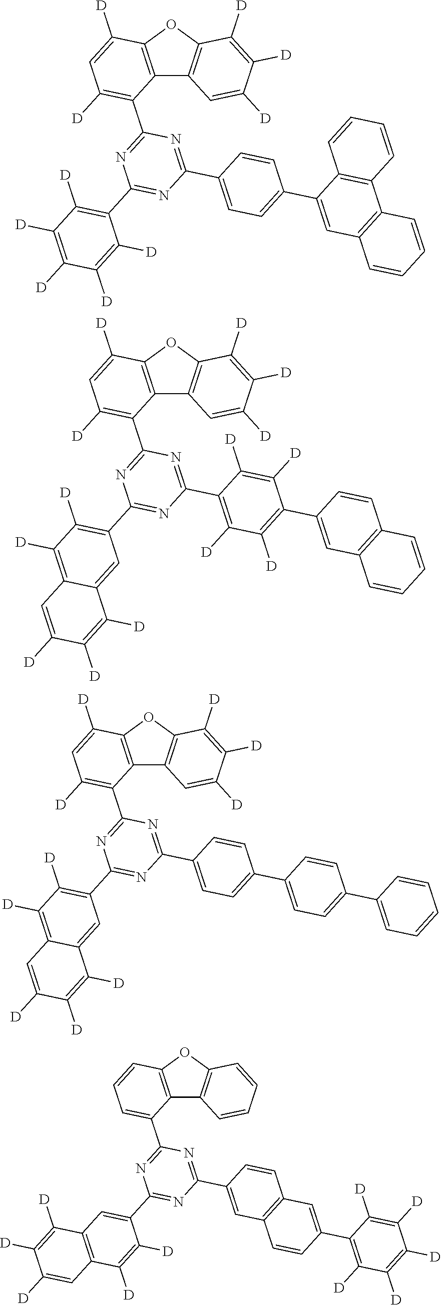

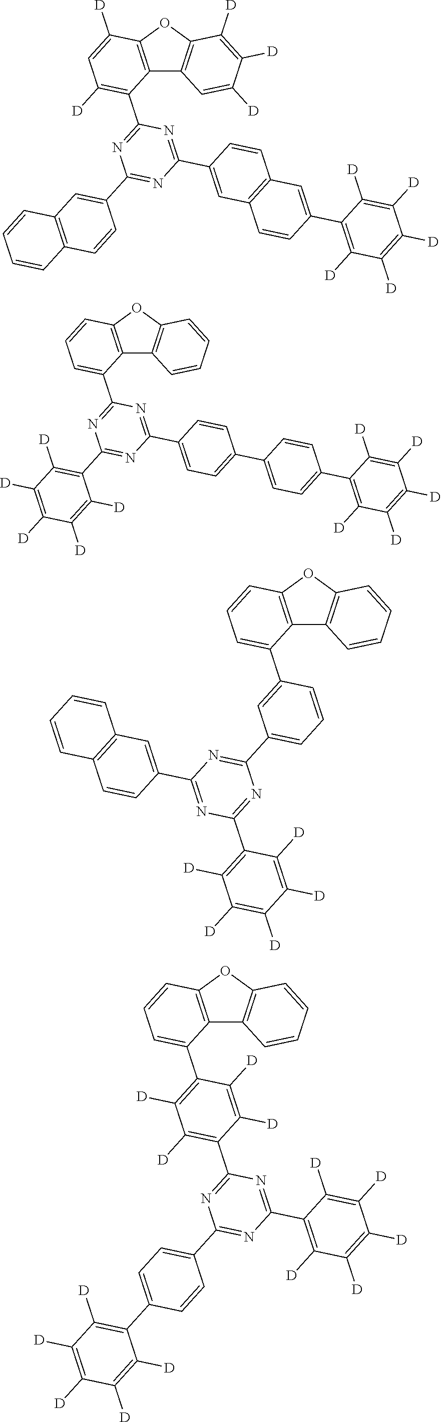

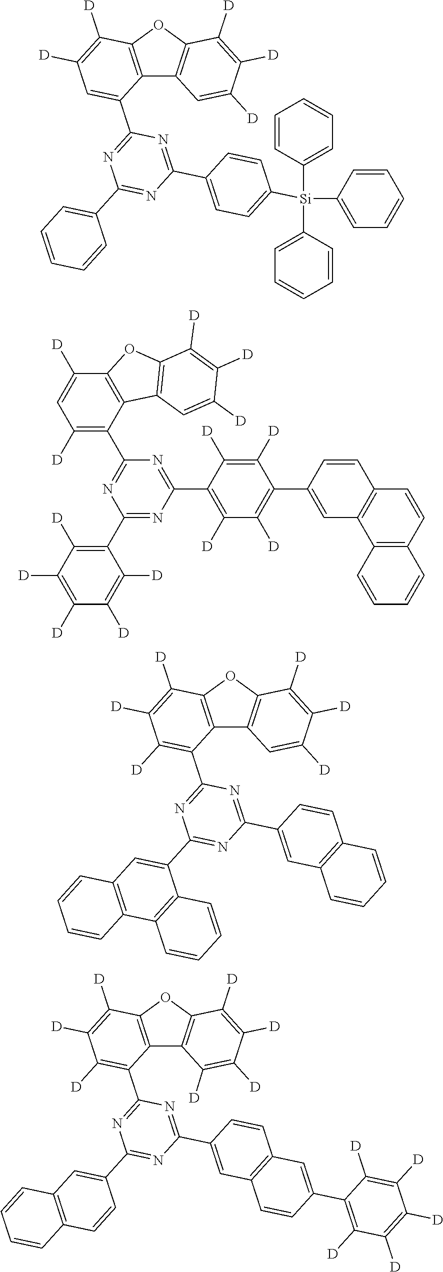

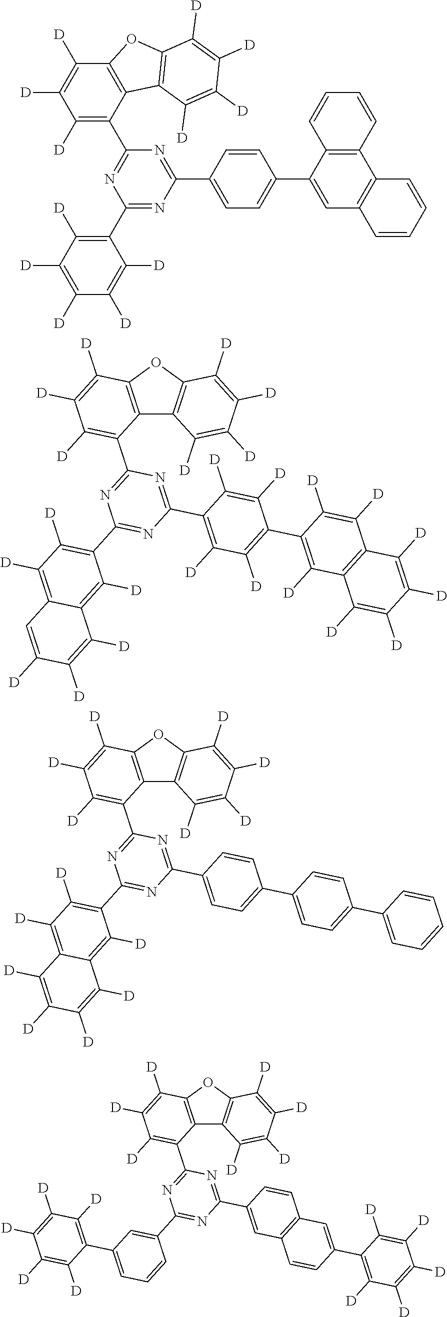

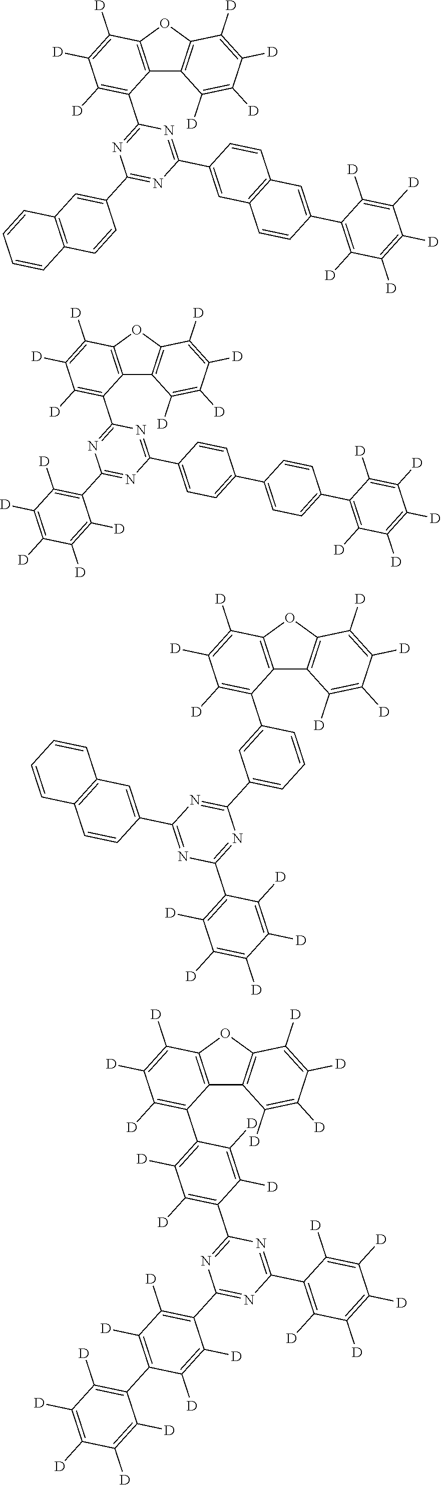

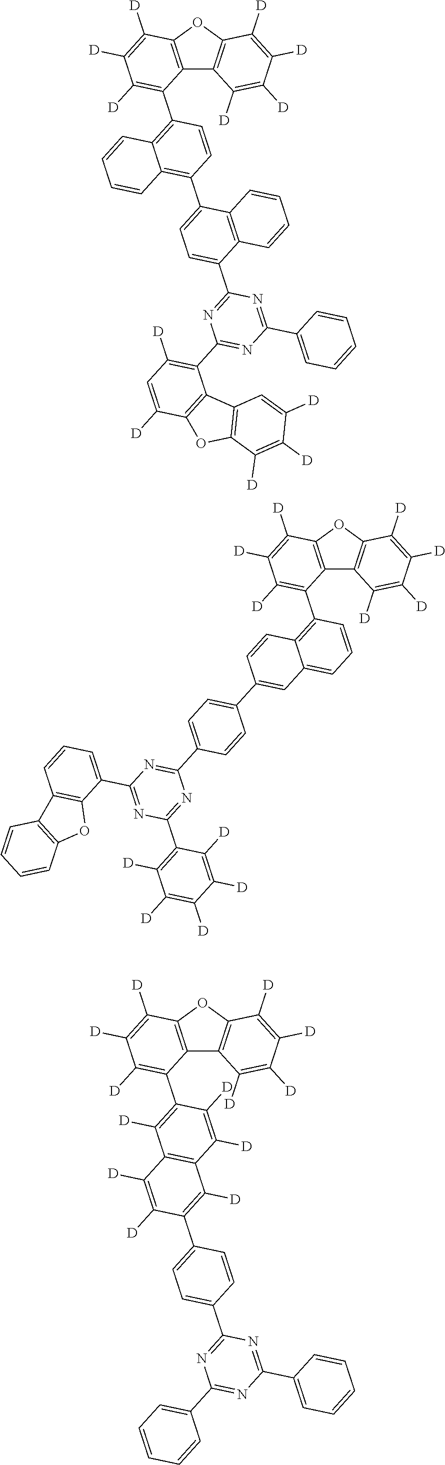

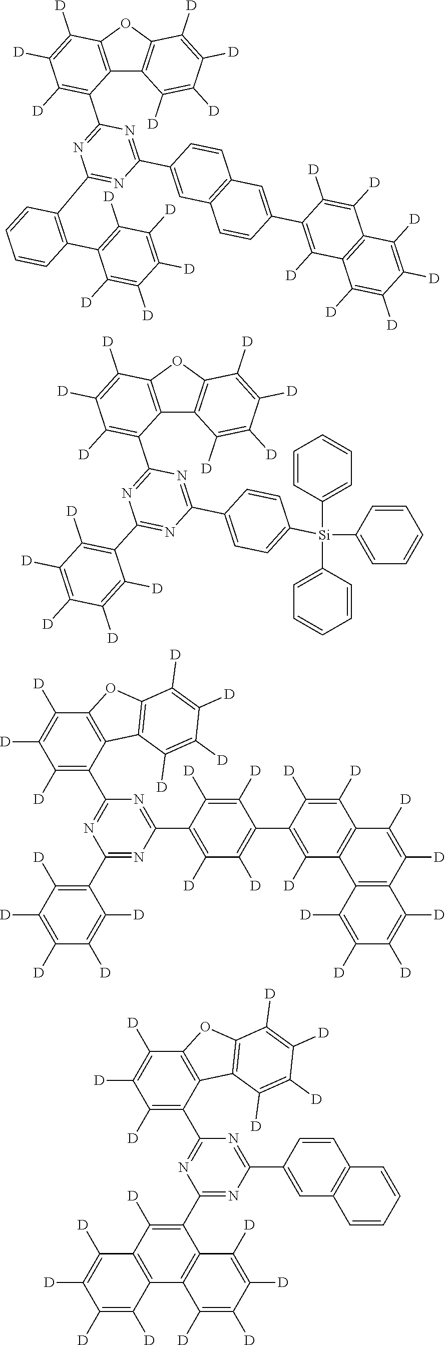

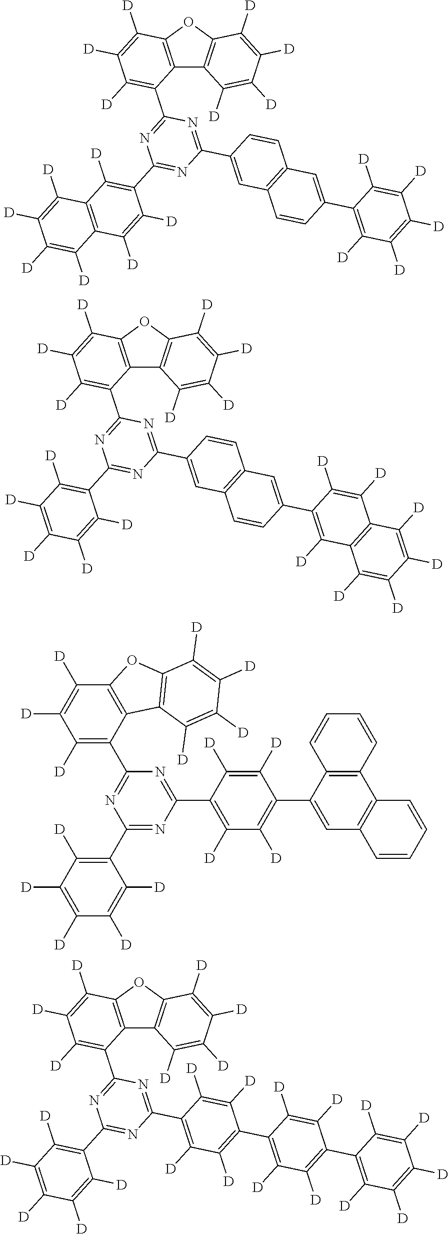

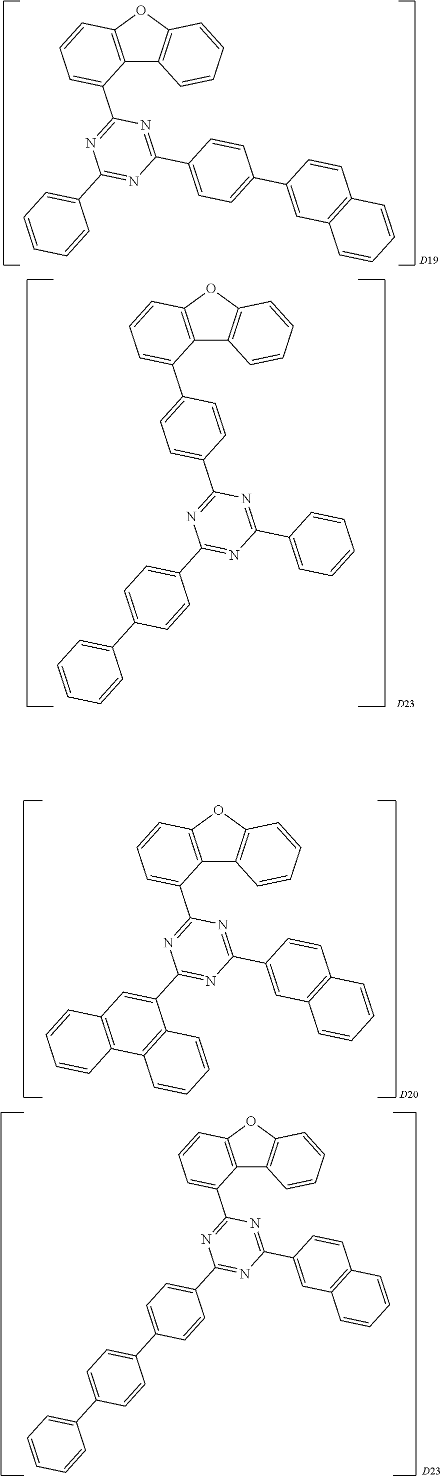

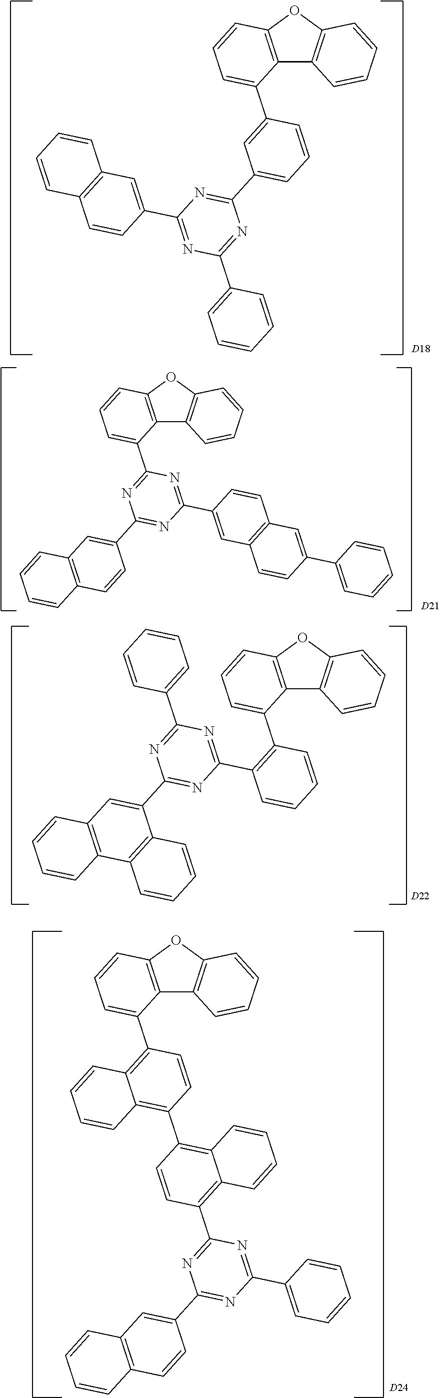

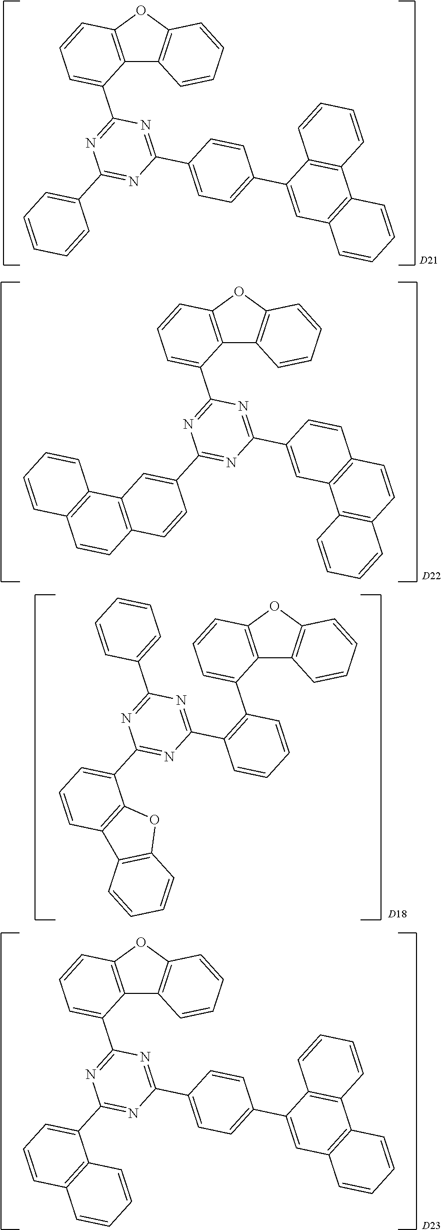

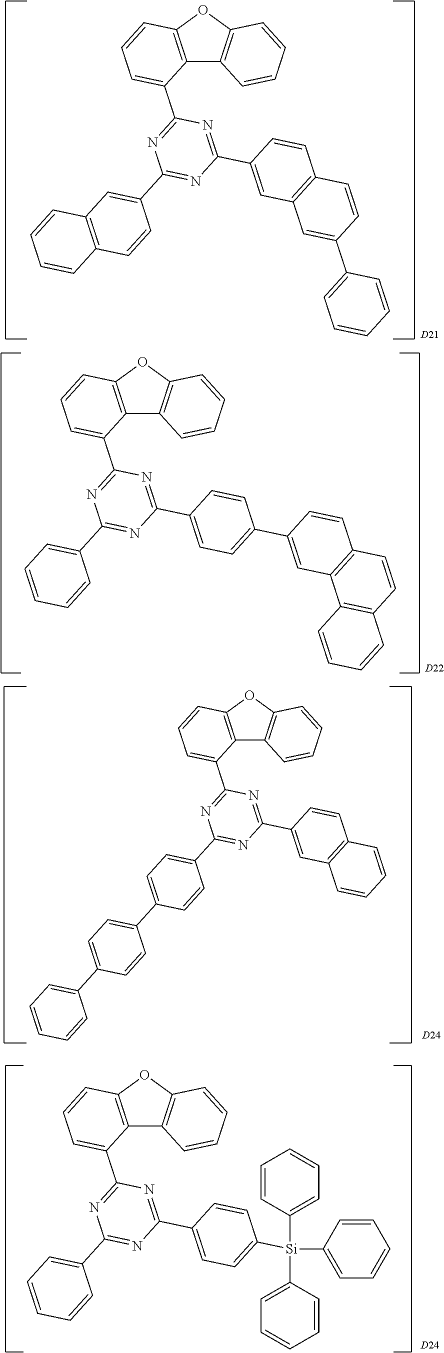

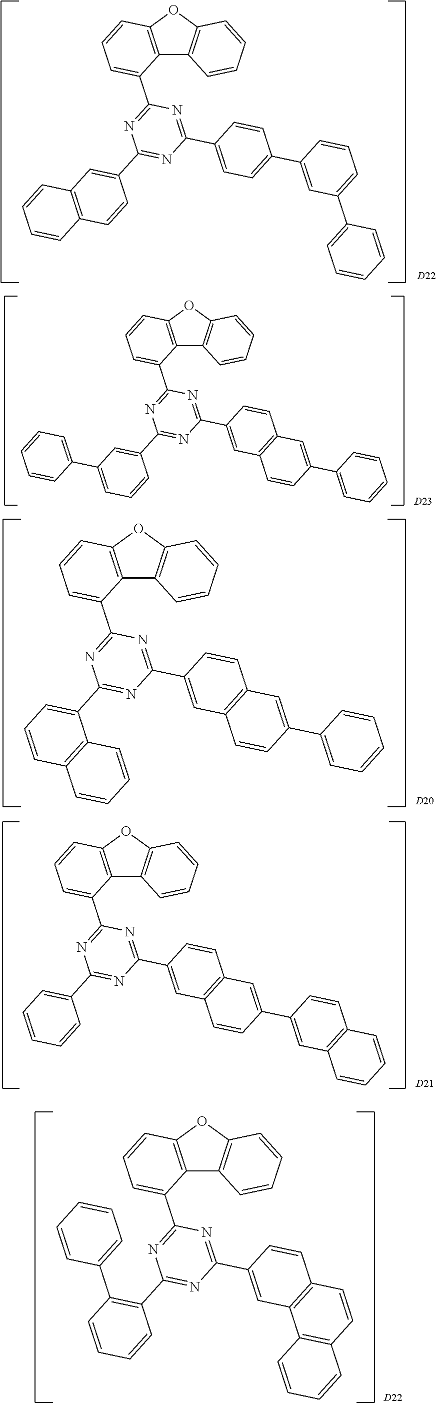

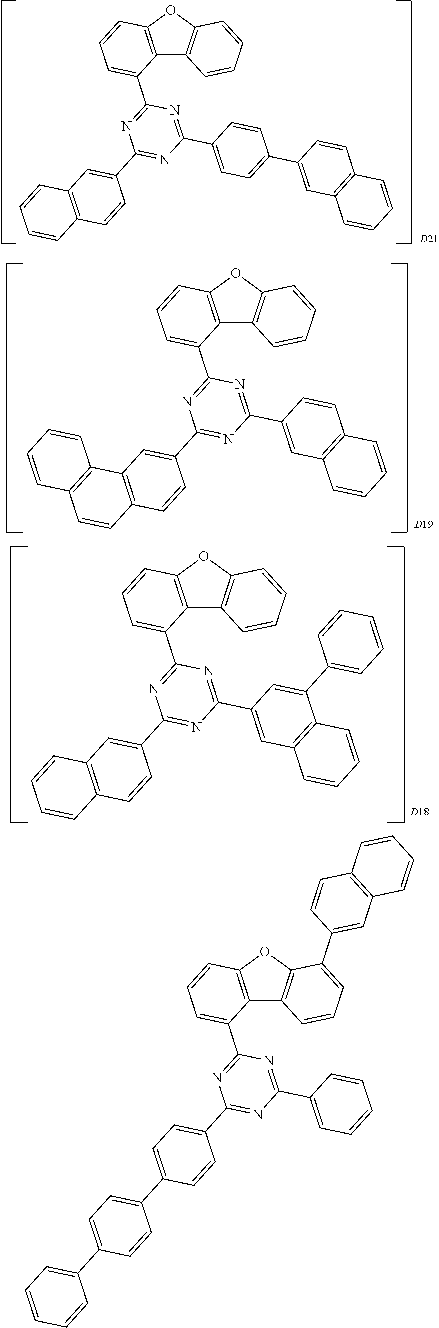

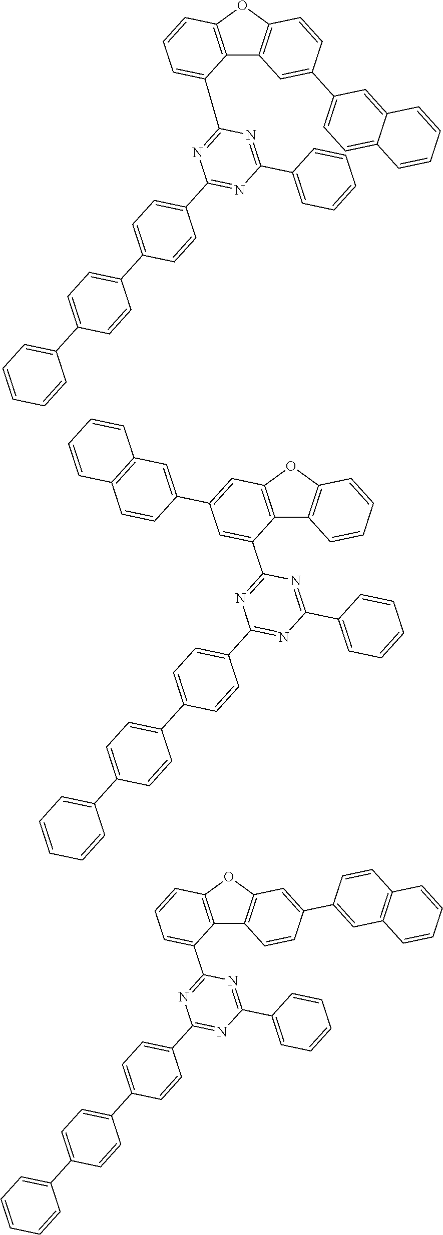

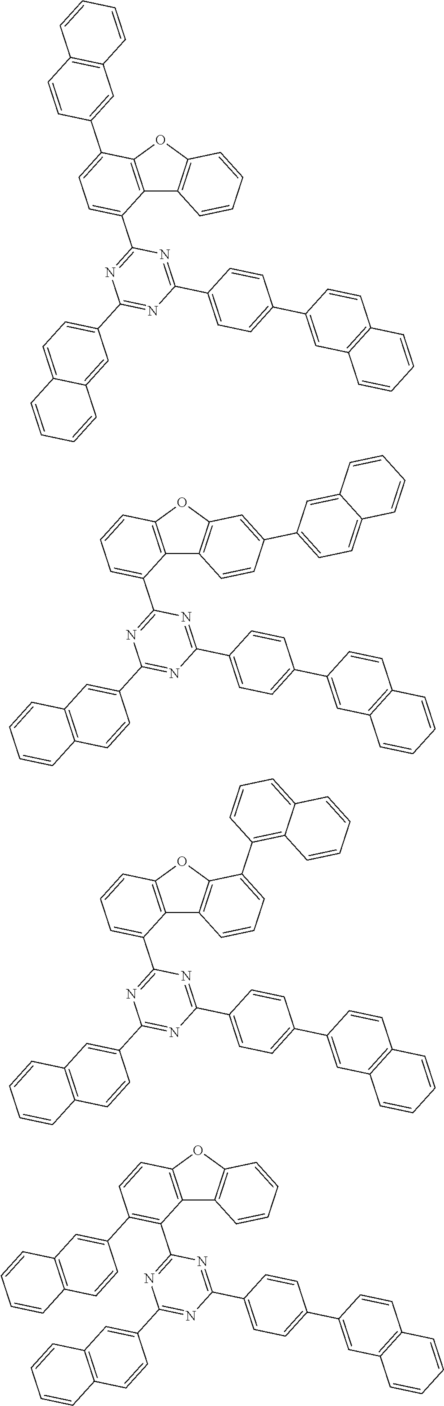 [SMILES: c1ccc2cc(-c3ccc(-c4nc(-c5ccc6ccccc6c5)nc(-c5ccc(-c6ccc7ccccc7c6)c6oc7ccccc7c56)n4)cc3)ccc2c1 ; c1ccc2cc(-c3ccc(-c4nc(-c5ccc6ccccc6c5)nc(-c5cccc6oc7cc(-c8ccc9ccccc9c8)ccc7c56)n4)cc3)ccc2c1 ; c1ccc2cc(-c3ccc(-c4nc(-c5ccc6ccccc6c5)nc(-c5cccc6oc7c(-c8cccc9ccccc89)cccc7c56)n4)cc3)ccc2c1 ; c1ccc2cc(-c3ccc(-c4nc(-c5ccc6ccccc6c5)nc(-c5c(-c6ccc7ccccc7c6)ccc6oc7ccccc7c56)n4)cc3)ccc2c1]

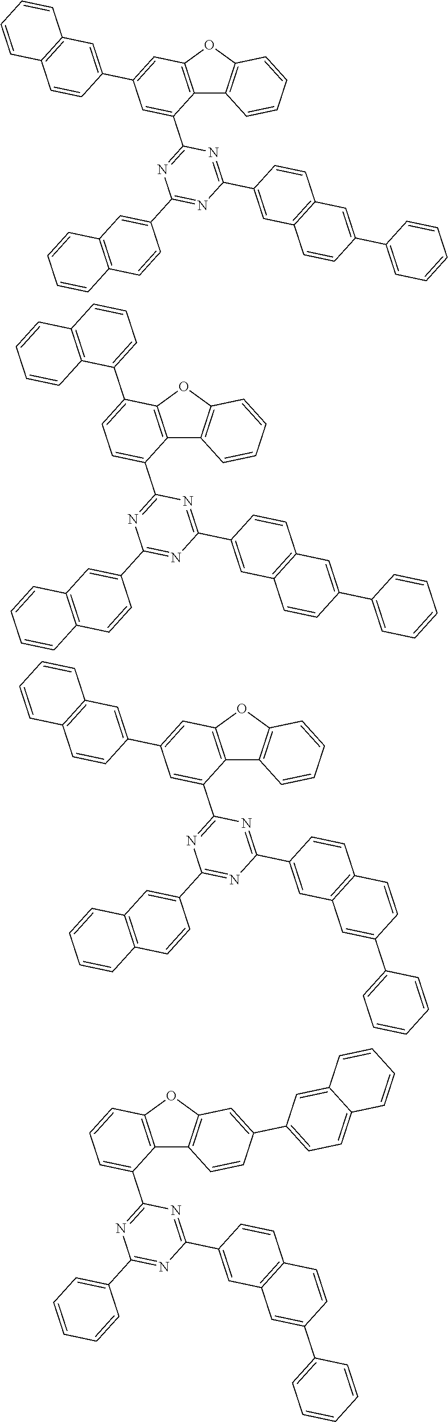

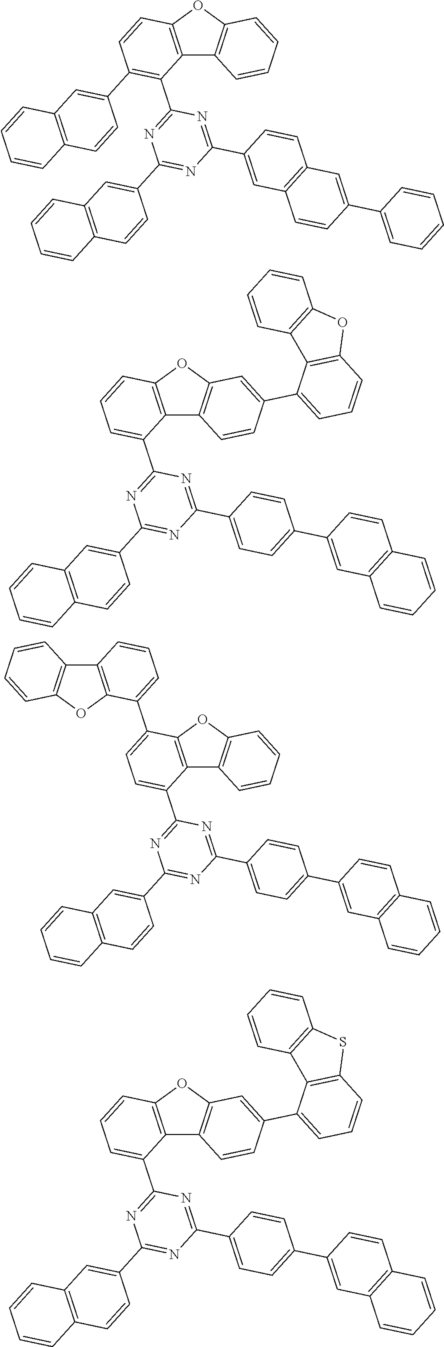

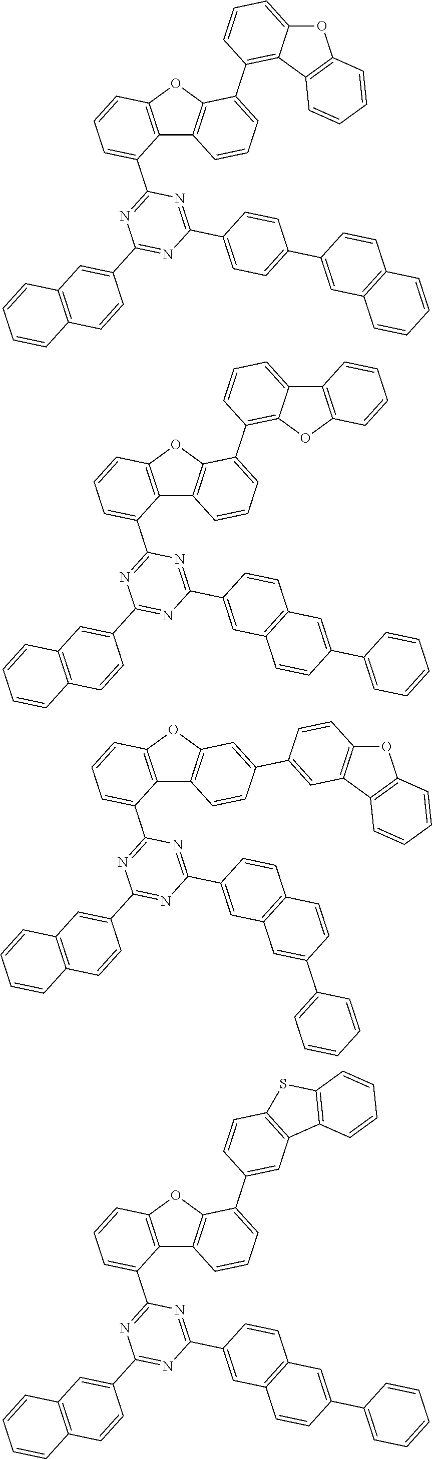

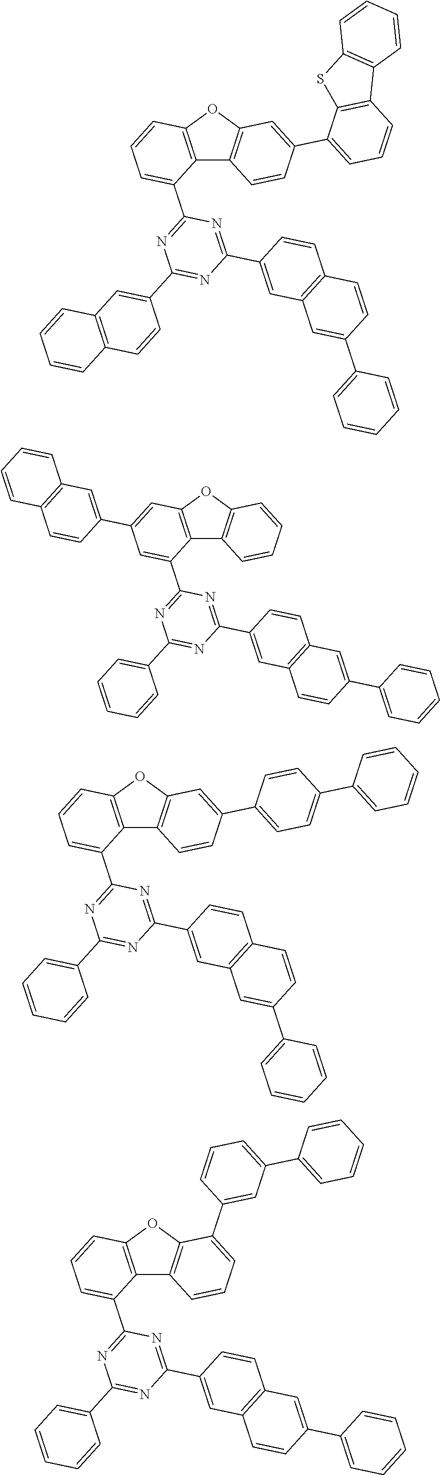

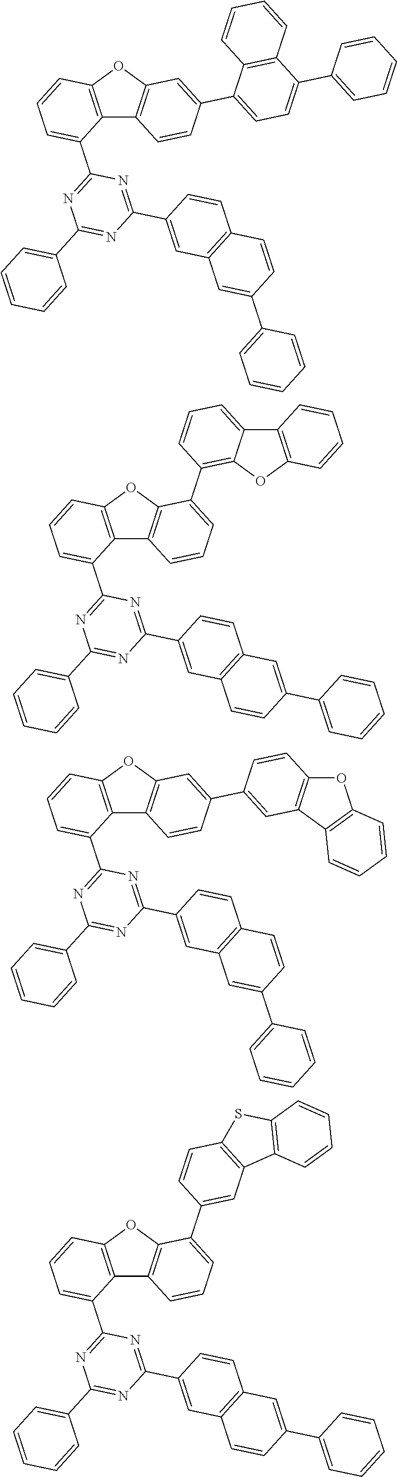

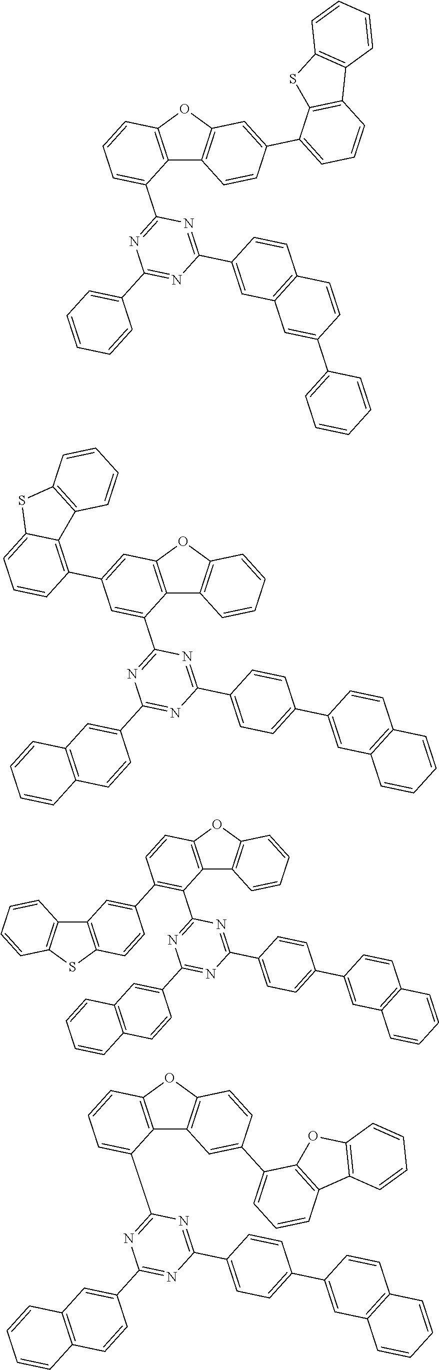

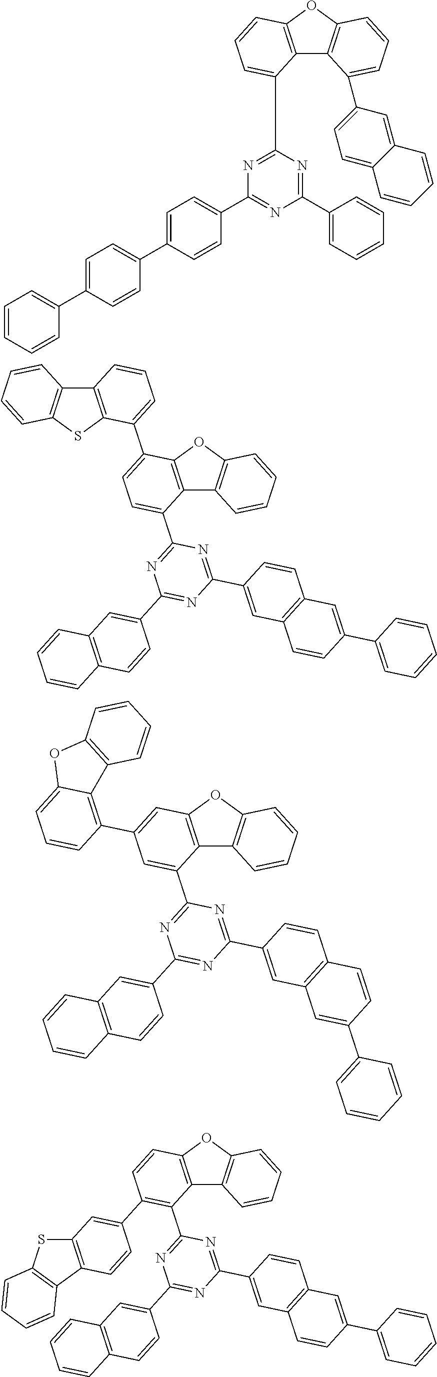

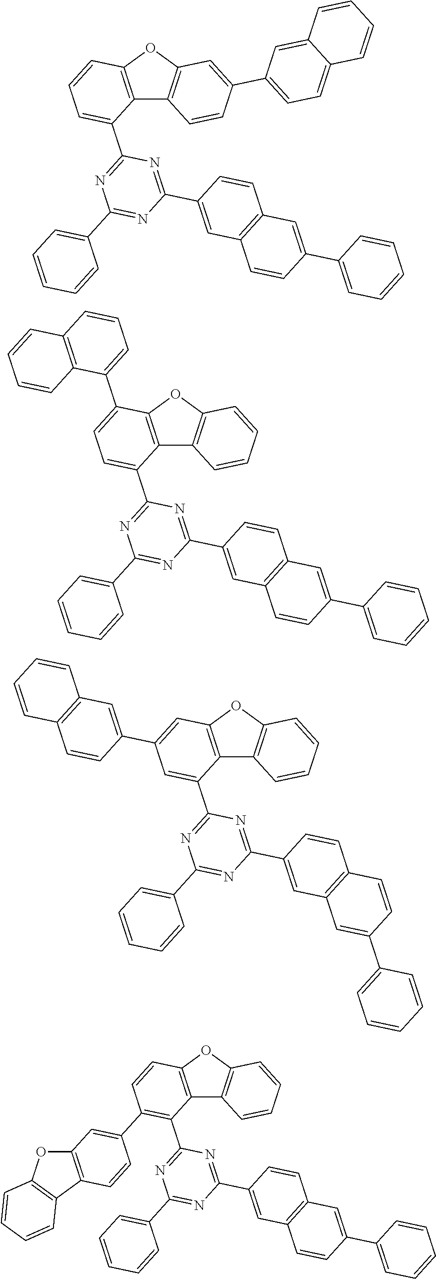

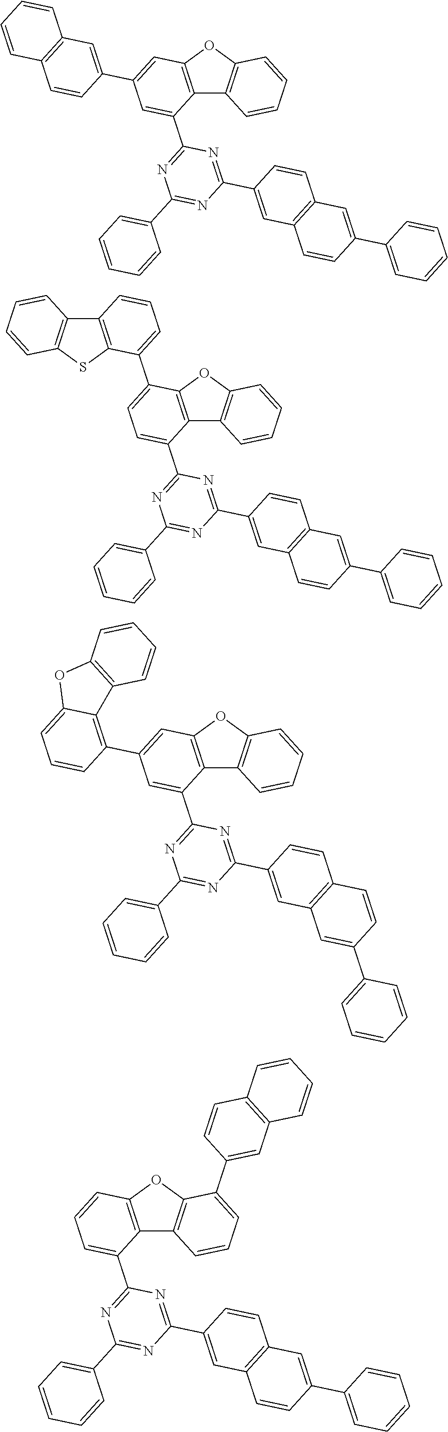

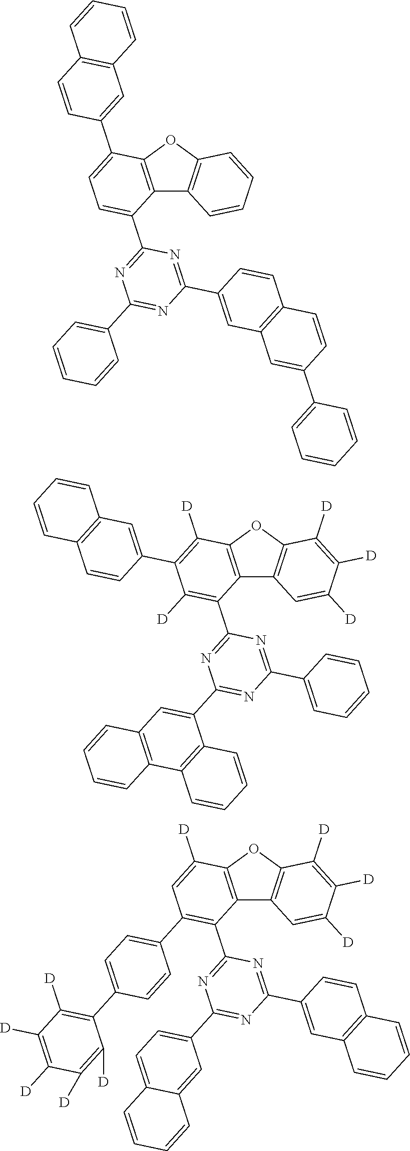

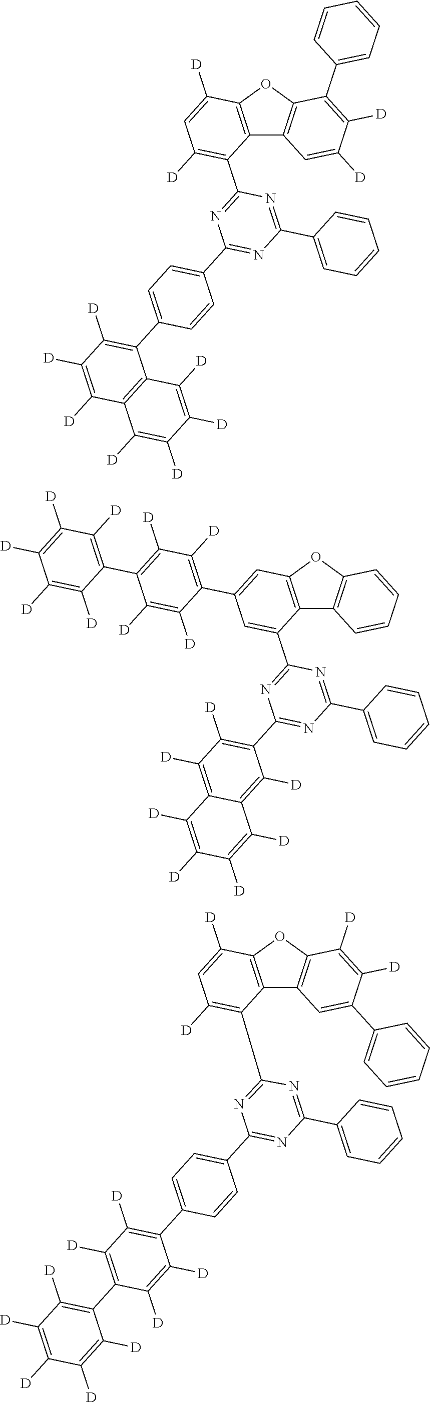

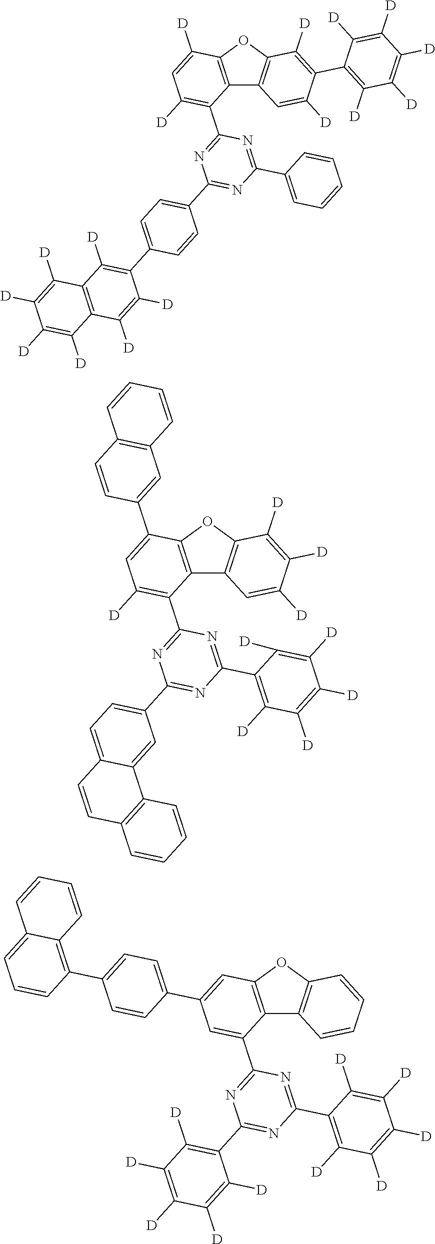

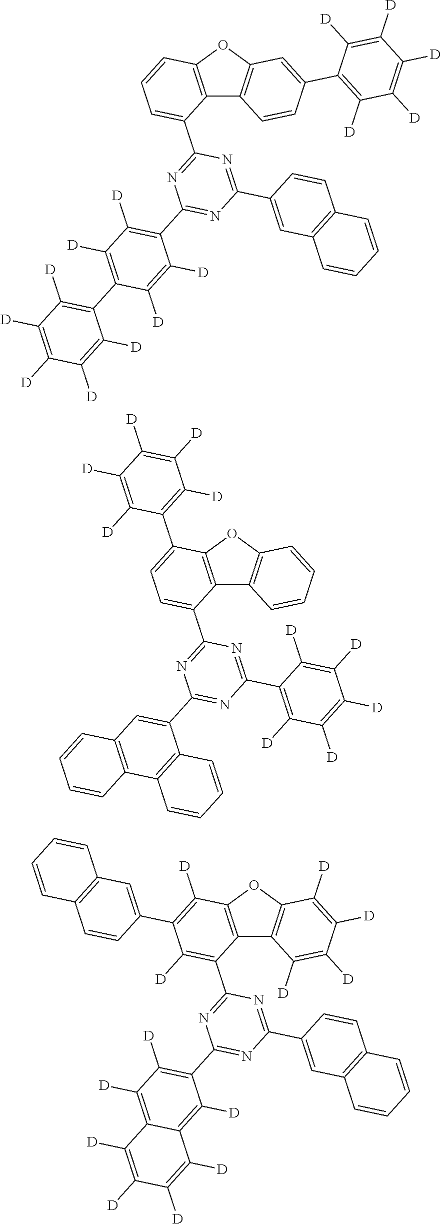

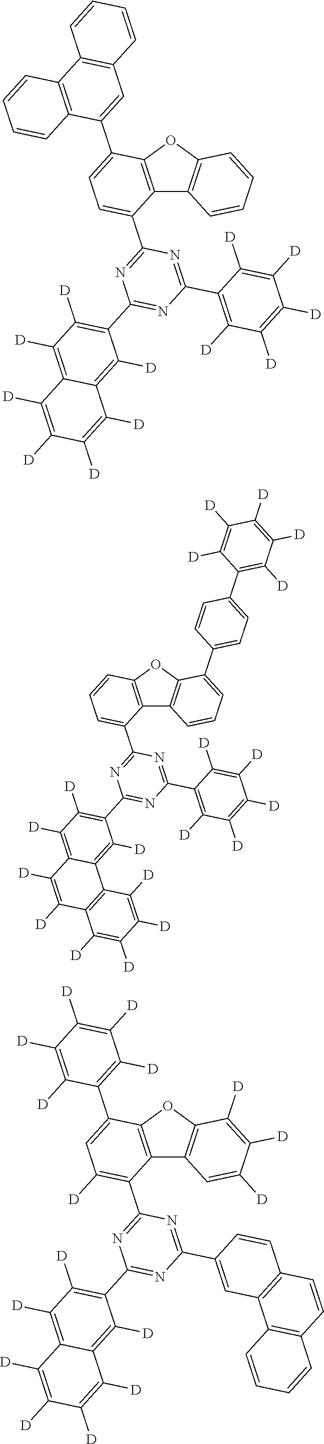

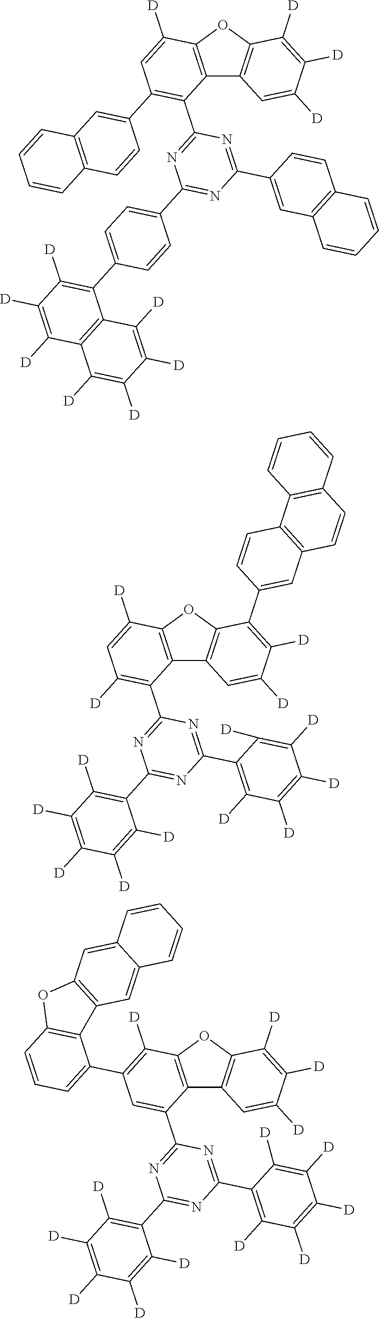

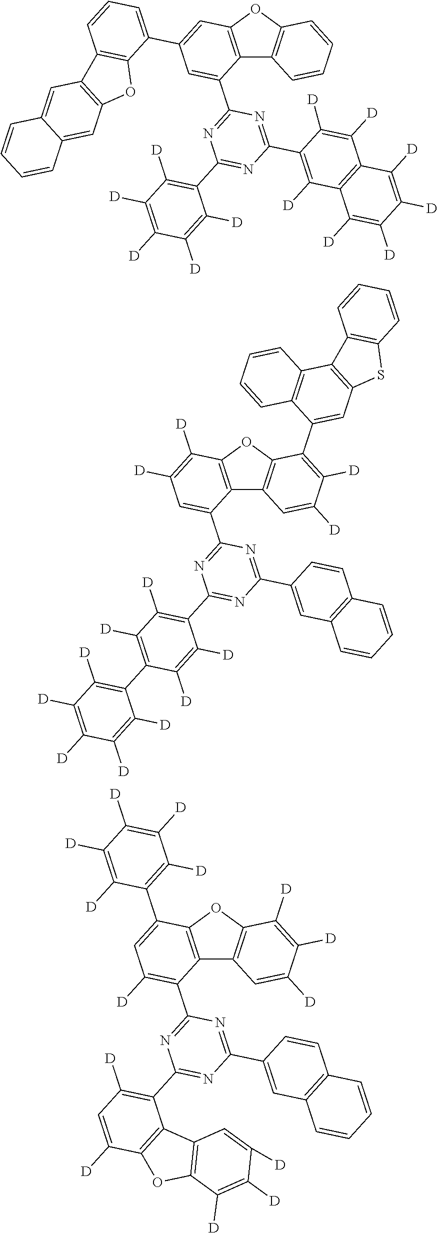

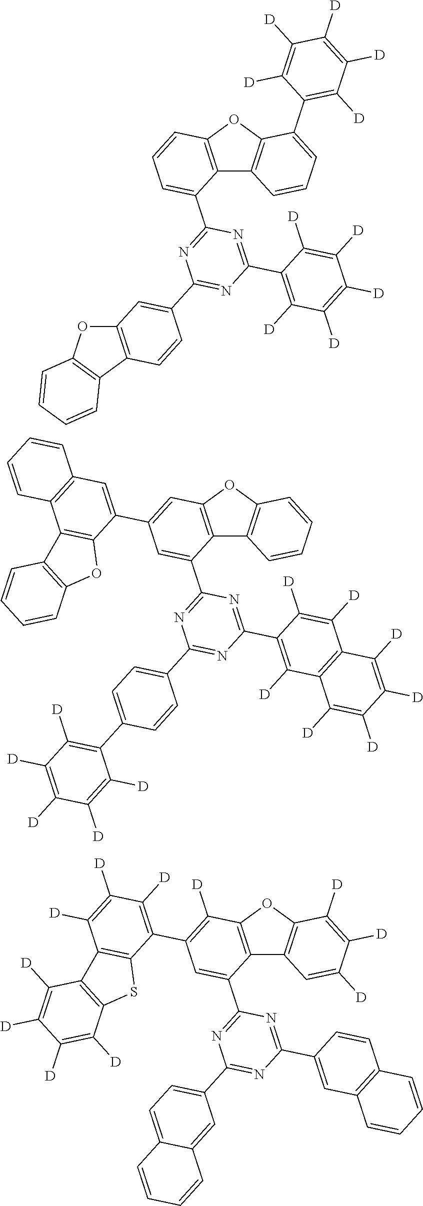

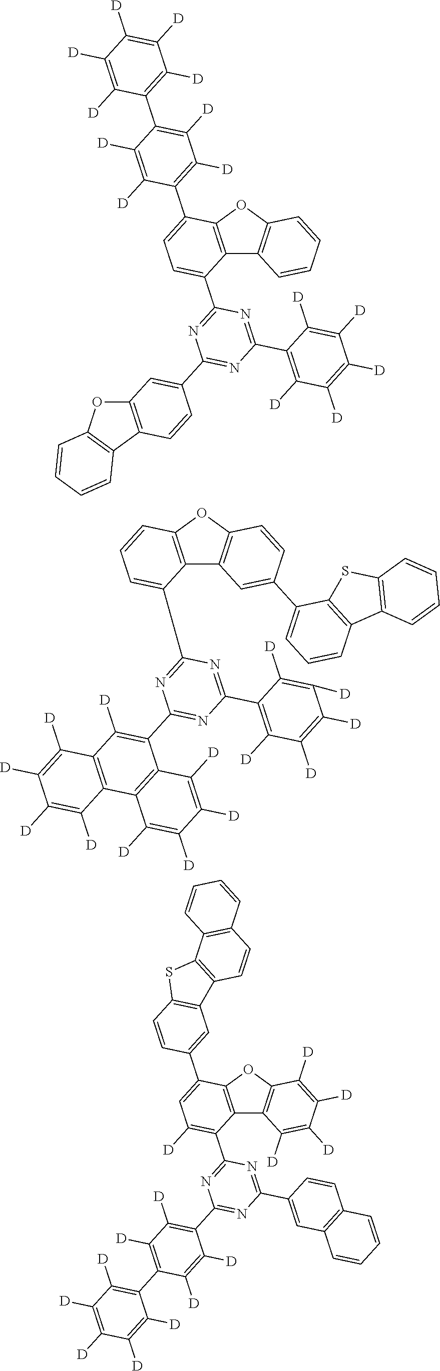

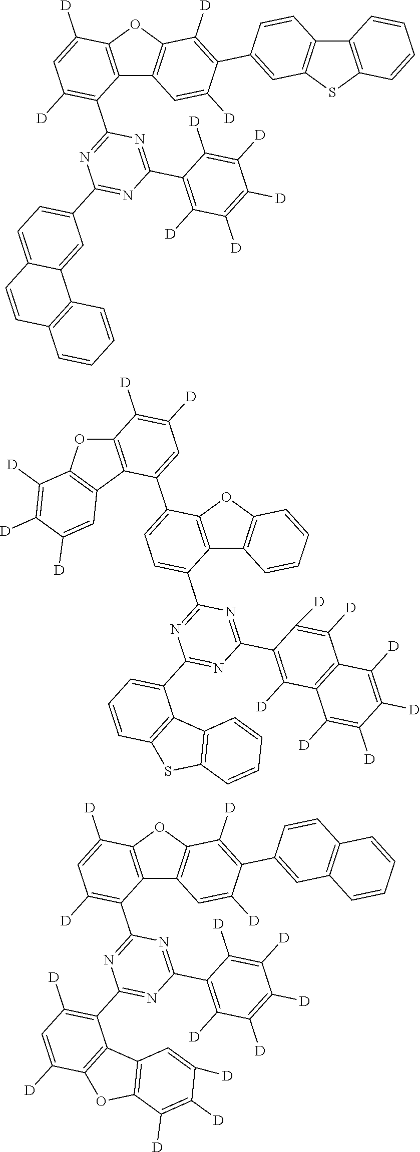

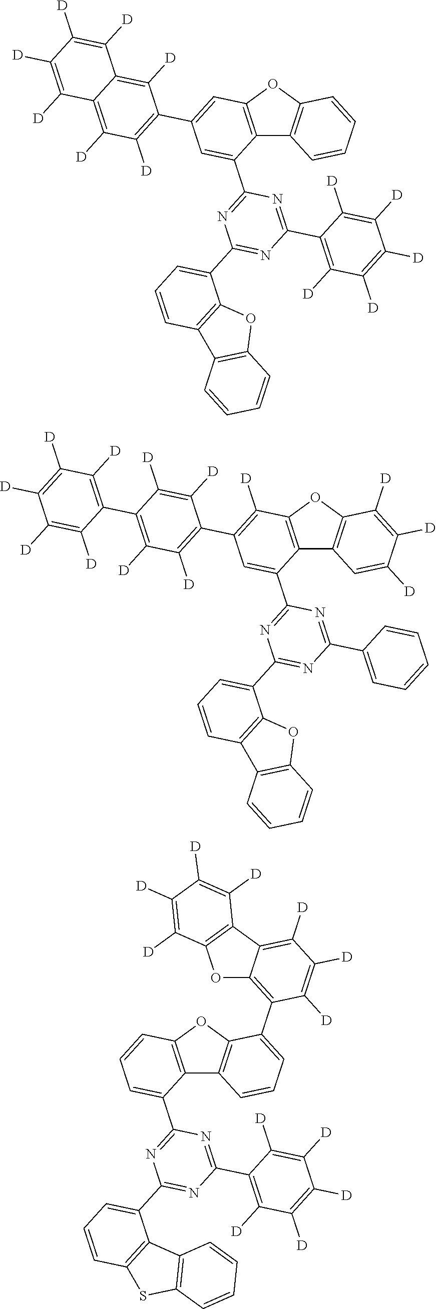 [SMILES: [2H]c1c([2H])c([2H])c(-c2nc(-c3cccc4c3oc3ccccc34)nc(-c3cc(-c4c([2H])c([2H])c5c([2H])c([2H])c([2H])c([2H])c5c4[2H])cc4oc5ccccc5c34)n2)c([2H])c1[2H] ; [2H]c1cc2c(oc3c([2H])c(-c4c([2H])c([2H])c(-c5c([2H])c([2H])c([2H])c([2H])c5[2H])c([2H])c4[2H])cc(-c4nc(-c5ccccc5)nc(-c5cccc6c5oc5ccccc56)n4)c32)c([2H])c1[2H] ; [2H]c1c([2H])c([2H])c(-c2nc(-c3cccc4oc5c(-c6c([2H])c([2H])c([2H])c7c6oc6c([2H])c([2H])c([2H])c([2H])c67)cccc5c34)nc(-c3cccc4sc5ccccc5c34)n2)c([2H])c1[2H]]

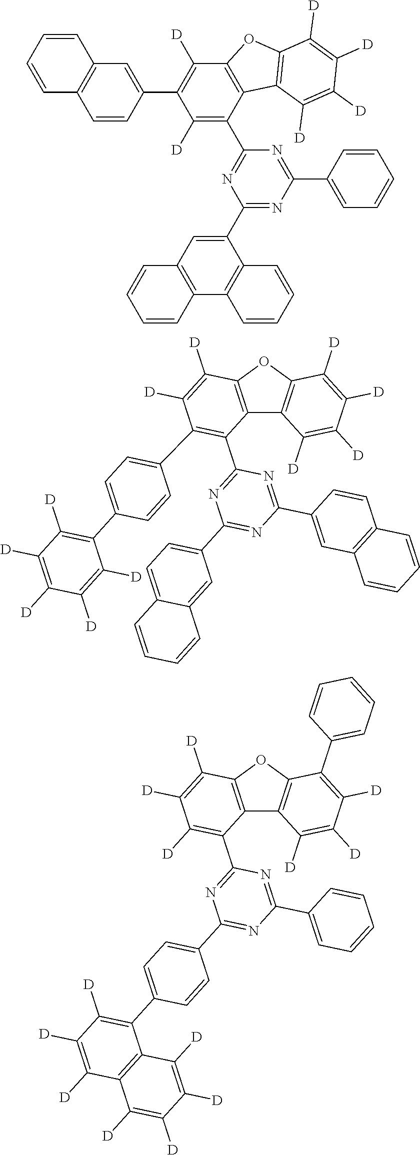

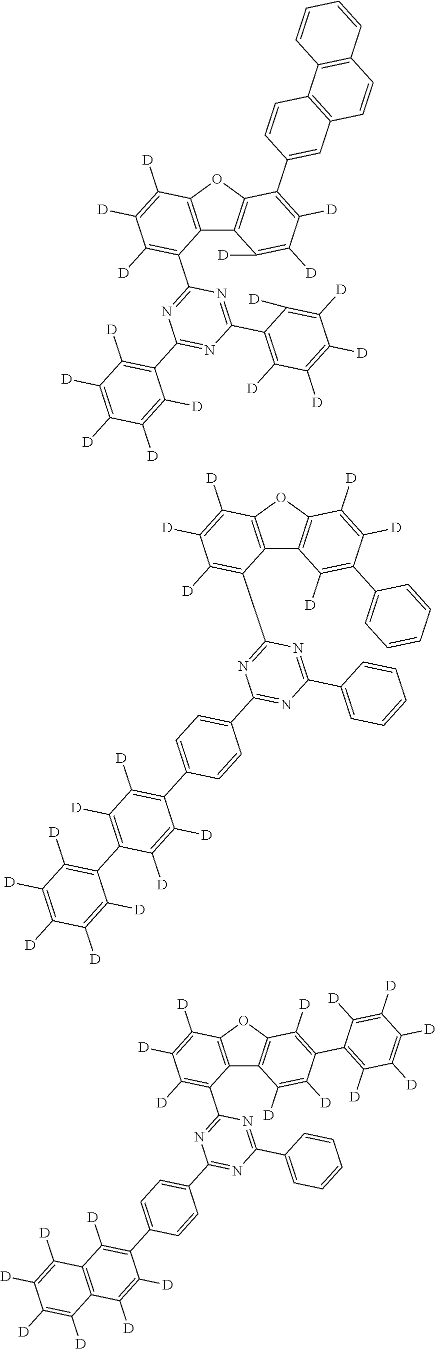

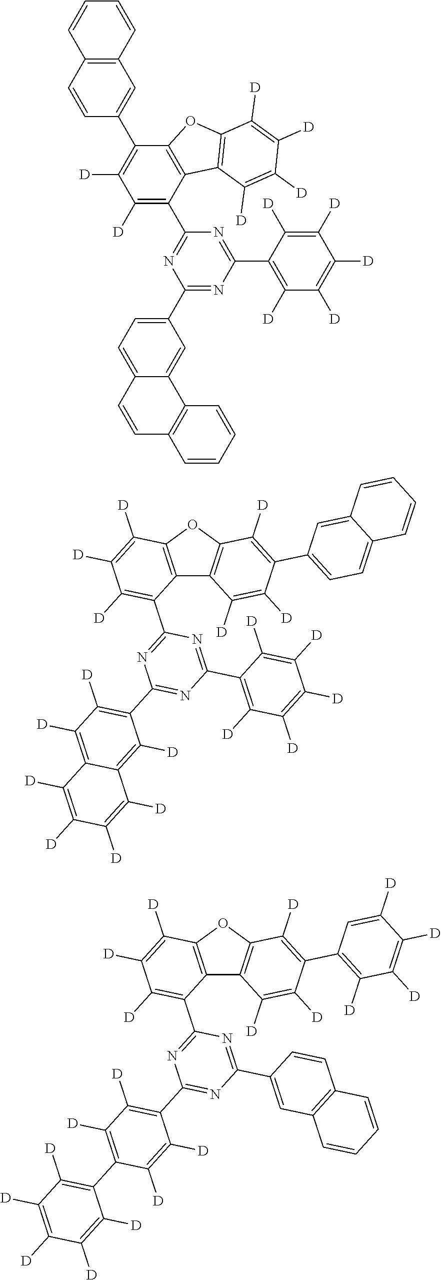

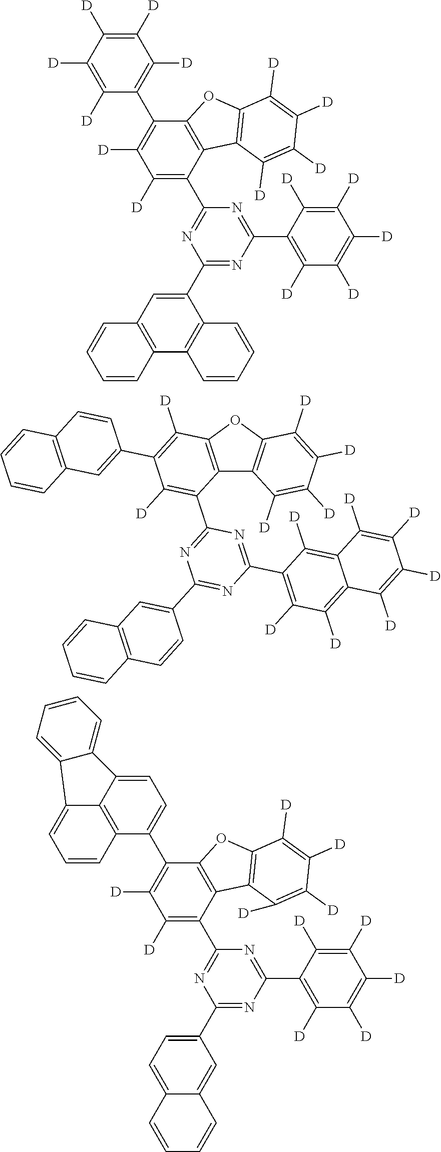

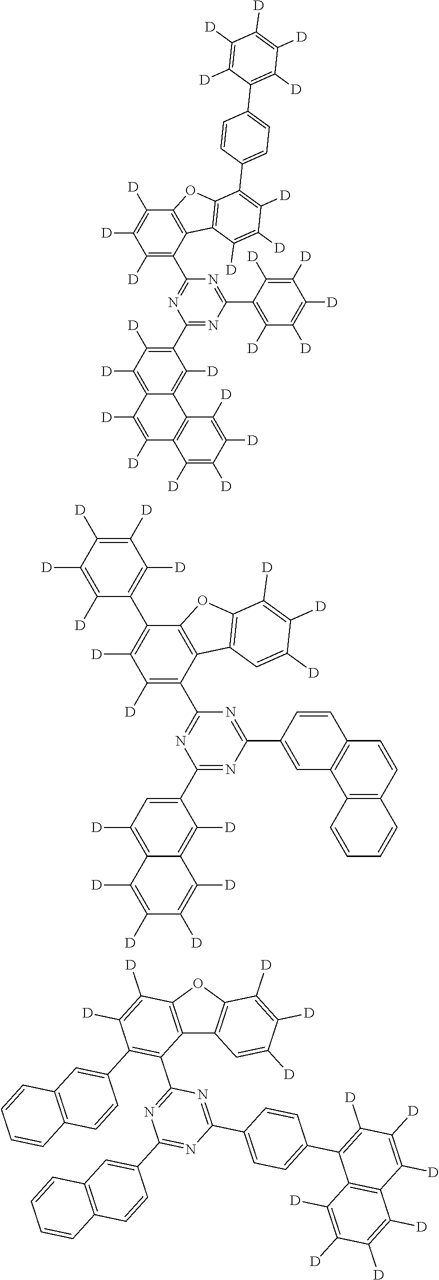

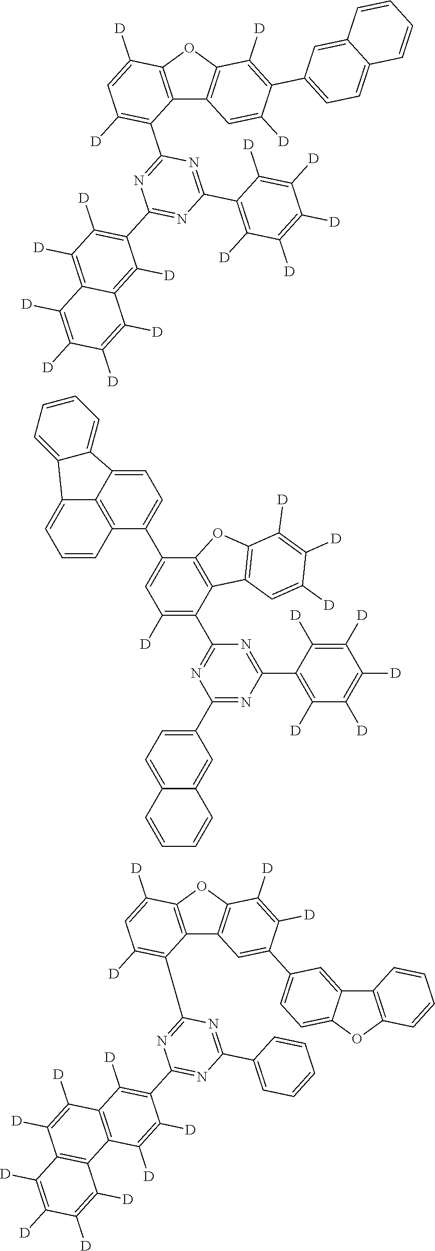

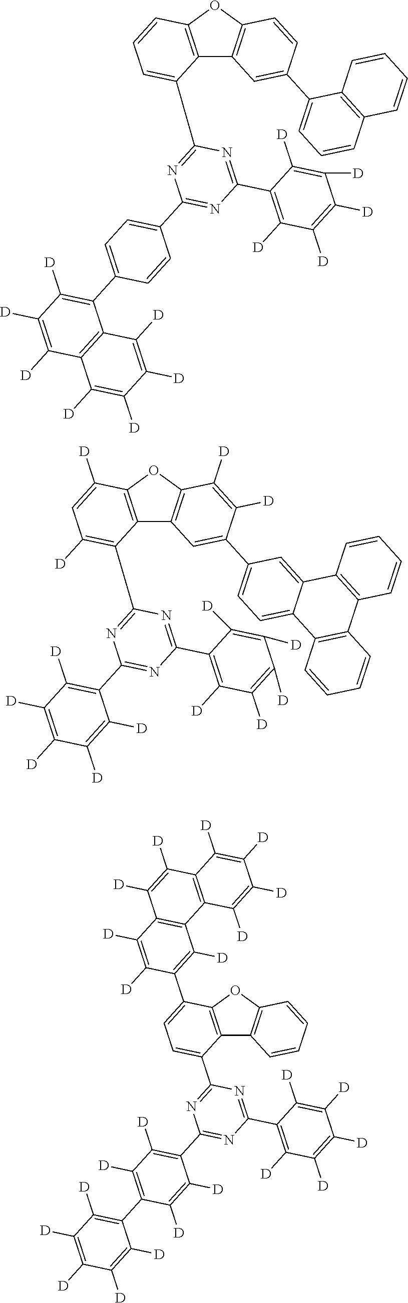

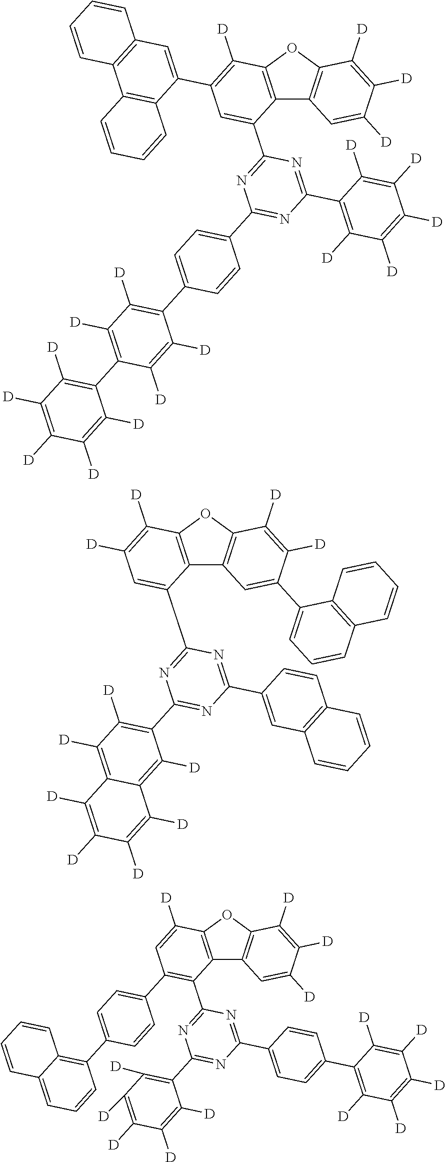

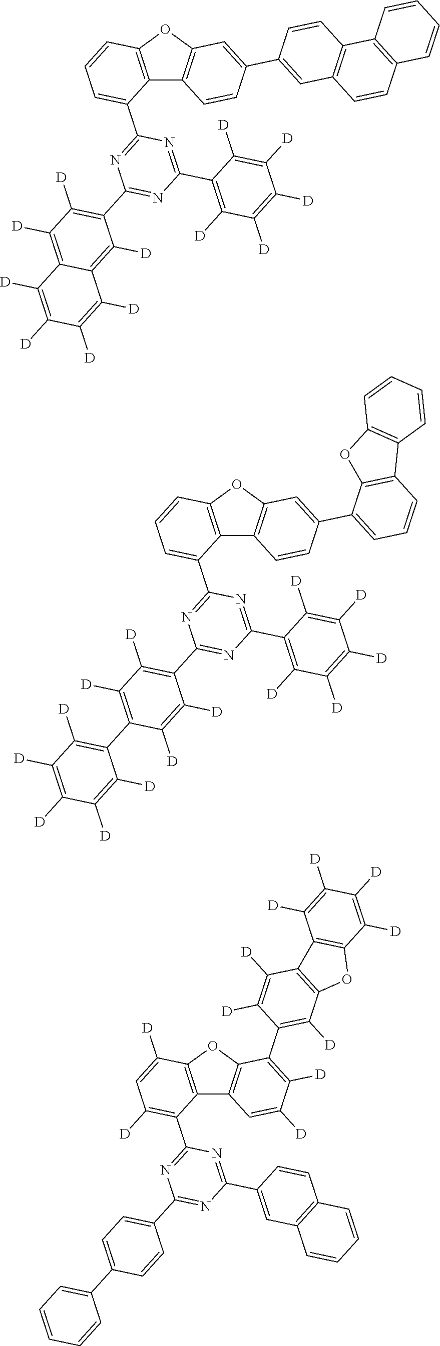 [SMILES: [2H]c1c([2H])c([2H])c(-c2nc(-c3c([2H])c([2H])c4c([2H])c([2H])c([2H])c([2H])c4c3[2H])nc(-c3cccc4oc5cc(-c6ccc7c(ccc8ccccc87)c6)ccc5c34)n2)c([2H])c1[2H] ; [2H]c1c([2H])c([2H])c(-c2nc(-c3c([2H])c([2H])c(-c4c([2H])c([2H])c([2H])c([2H])c4[2H])c([2H])c3[2H])nc(-c3cccc4oc5cc(-c6cccc7c6oc6ccccc67)ccc5c34)n2)c([2H])c1[2H] ; [2H]c1cc2c(oc3c([2H])cc([2H])c(-c4nc(-c5ccc(-c6ccccc6)cc5)nc(-c5ccc6ccccc6c5)n4)c32)c(-c2c([2H])c([2H])c3c(oc4c([2H])c([2H])c([2H])c([2H])c43)c2[2H])c1[2H]]

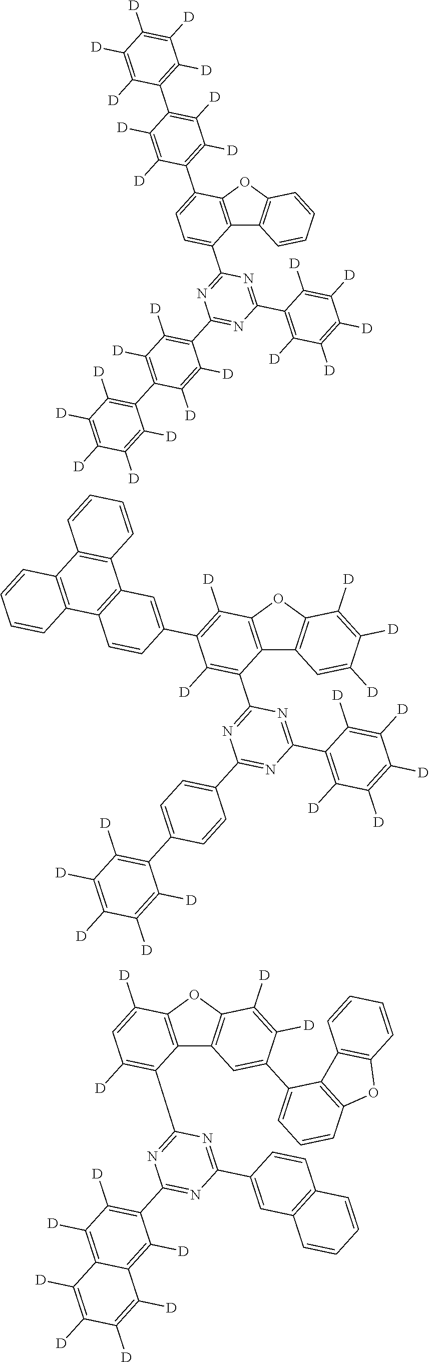

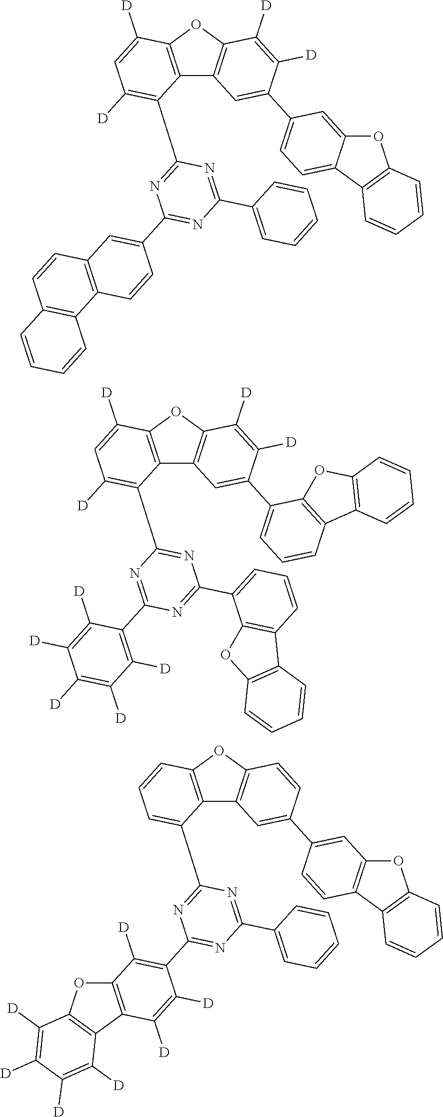

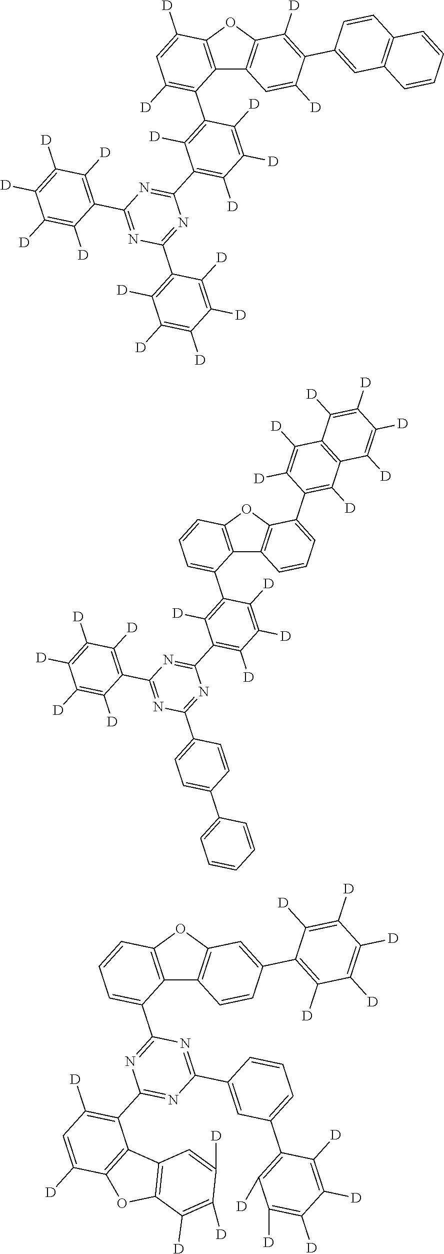

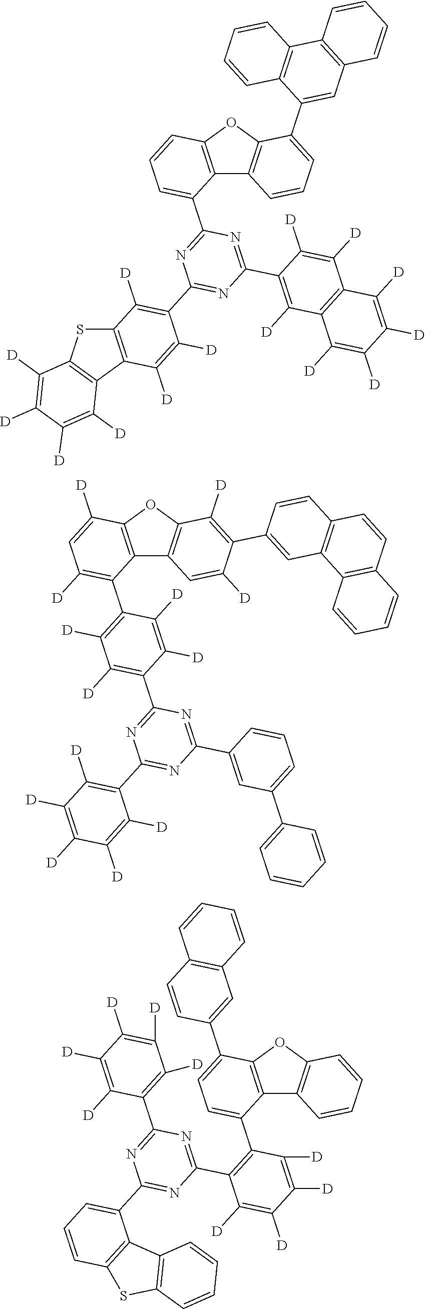

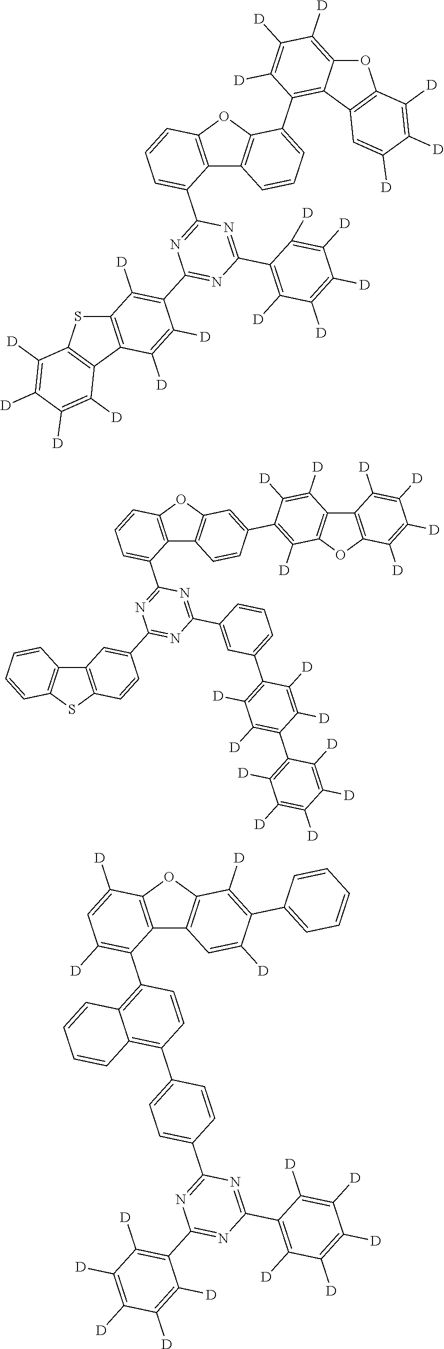

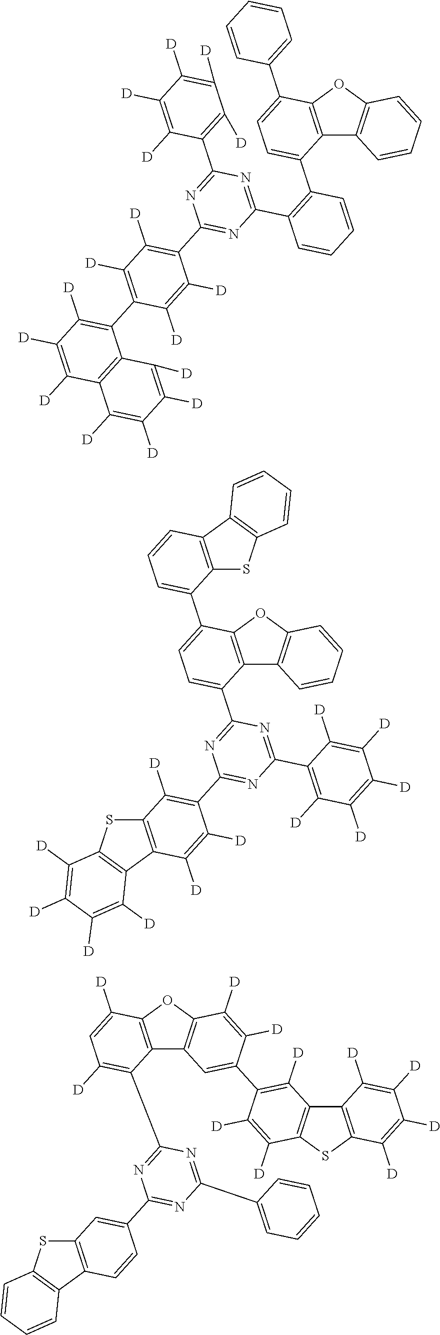

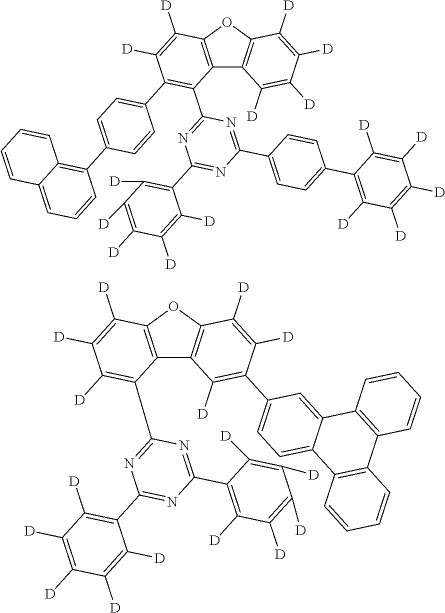

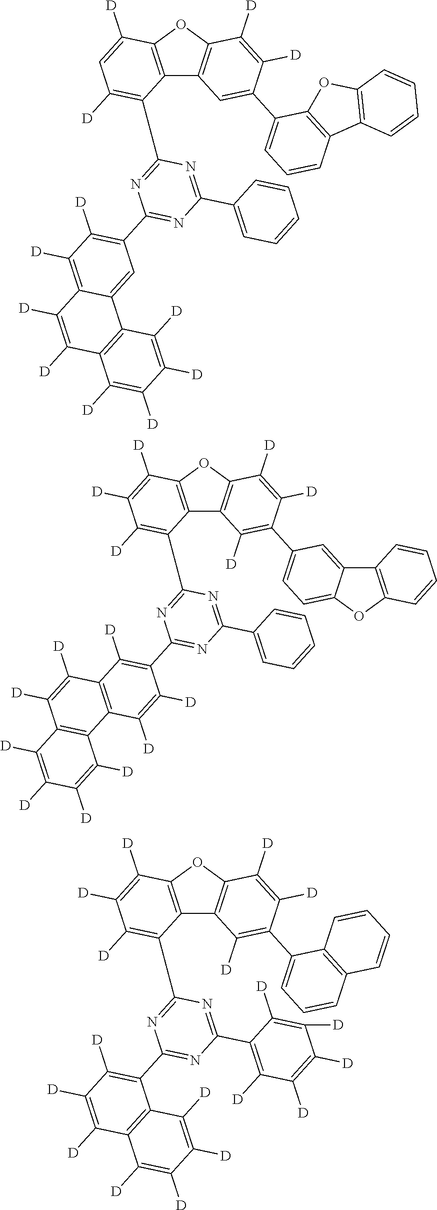

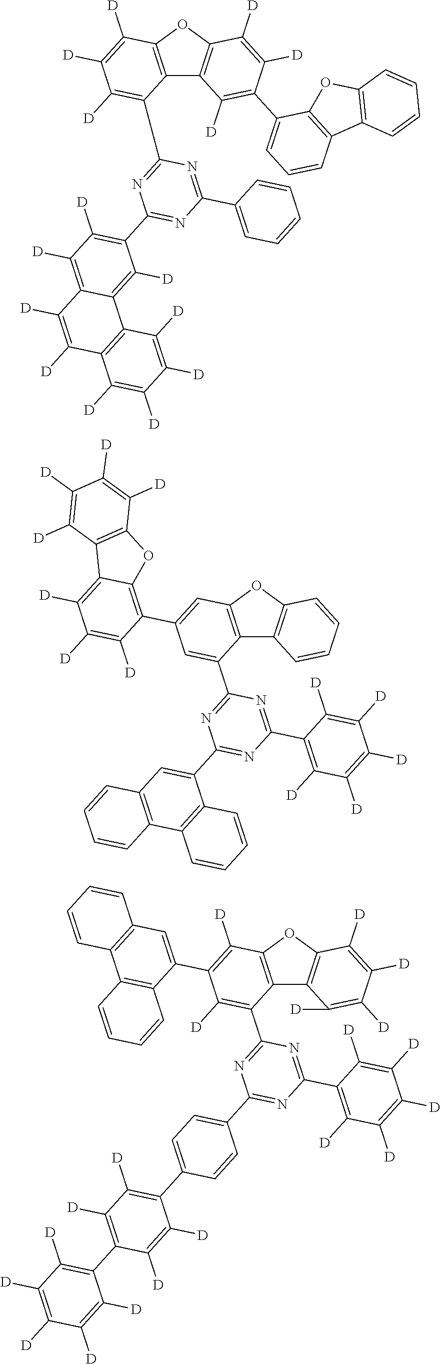

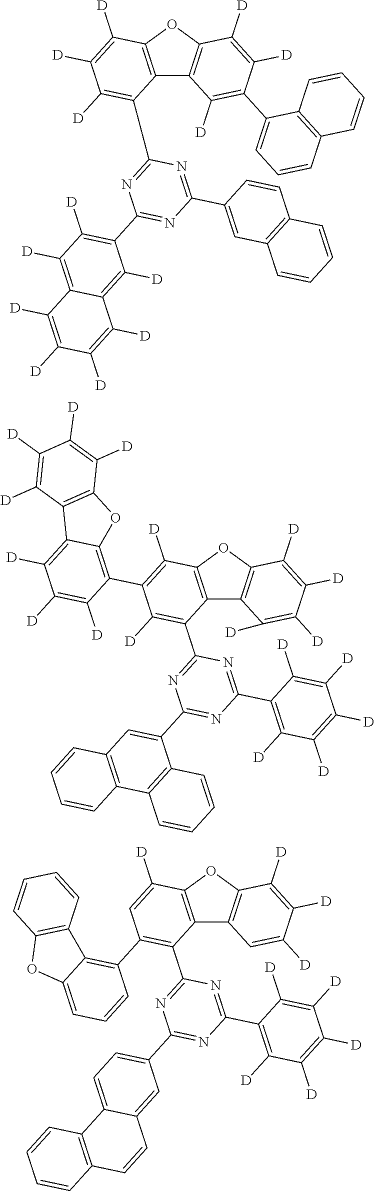

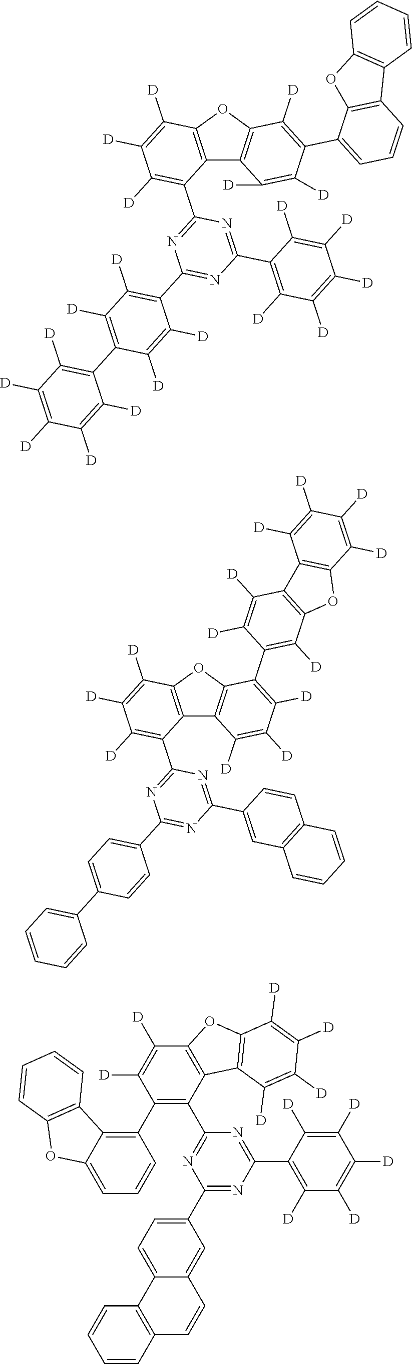

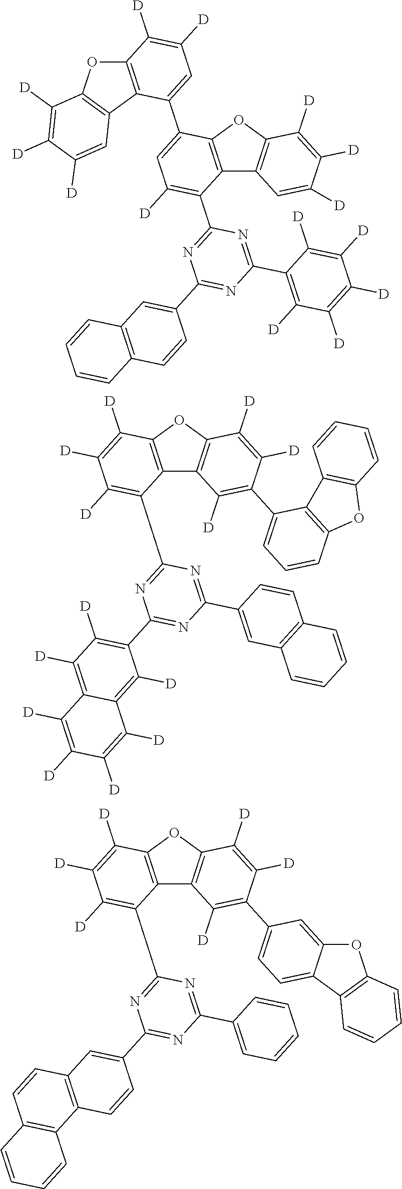

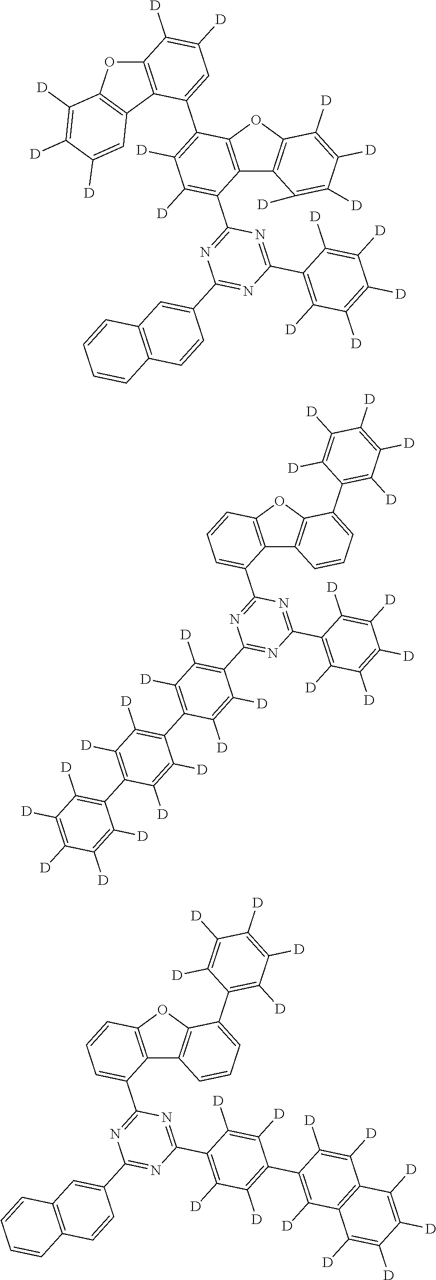

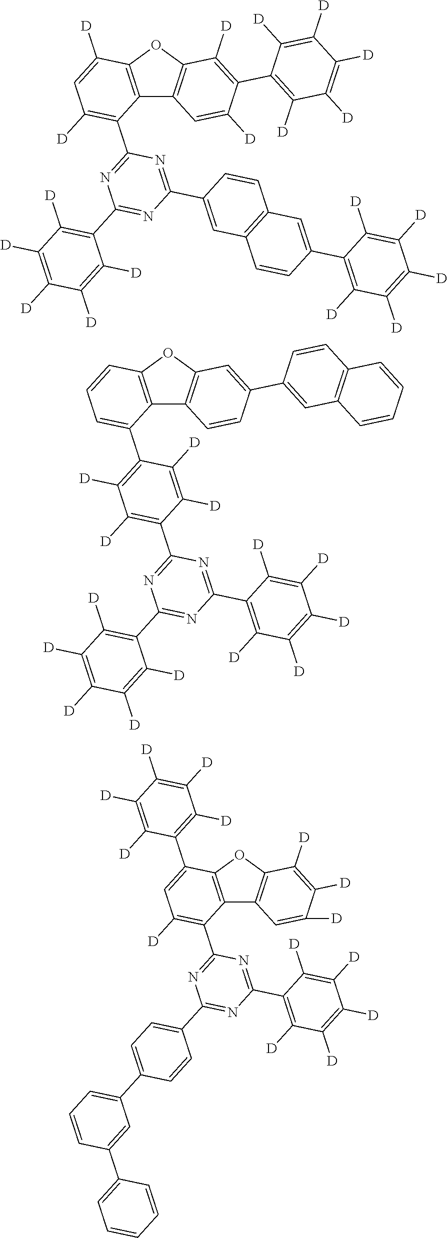

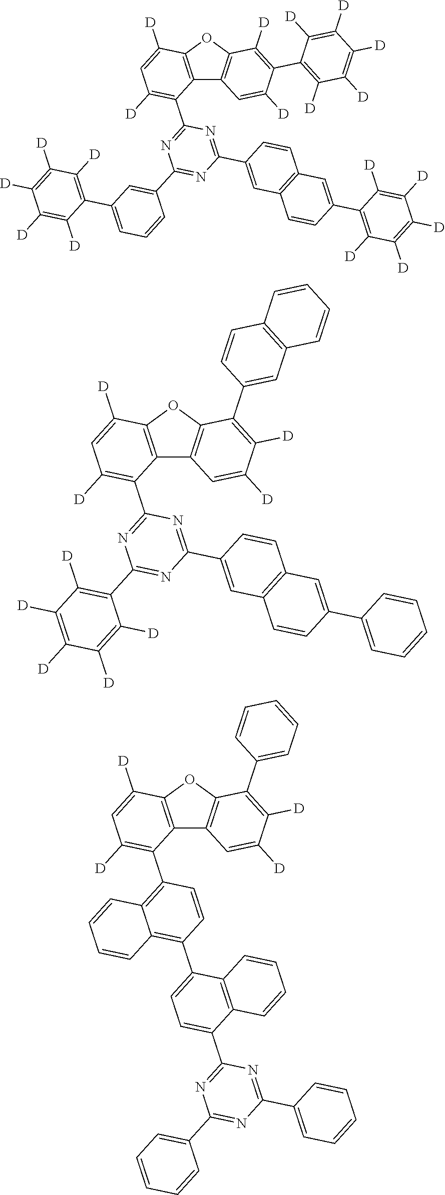 [SMILES: [2H]c1cc2c(oc3c([2H])cc([2H])c(-c4nc(-c5cccc(-c6c([2H])c([2H])c([2H])c([2H])c6[2H])c5)nc(-c5ccc6cc(-c7c([2H])c([2H])c([2H])c([2H])c7[2H])ccc6c5)n4)c32)c([2H])c1-c1c([2H])c([2H])c([2H])c([2H])c1[2H] ; [2H]c1cc2c(oc3c([2H])cc([2H])c(-c4nc(-c5ccc6cc(-c7ccccc7)ccc6c5)nc(-c5c([2H])c([2H])c([2H])c([2H])c5[2H])n4)c32)c(-c2ccc3ccccc3c2)c1[2H] ; [2H]c1cc2c(oc3c([2H])cc([2H])c(-c4ccc(-c5ccc(-c6nc(-c7ccccc7)nc(-c7ccccc7)n6)c6ccccc56)c5ccccc45)c32)c(-c2ccccc2)c1[2H]]

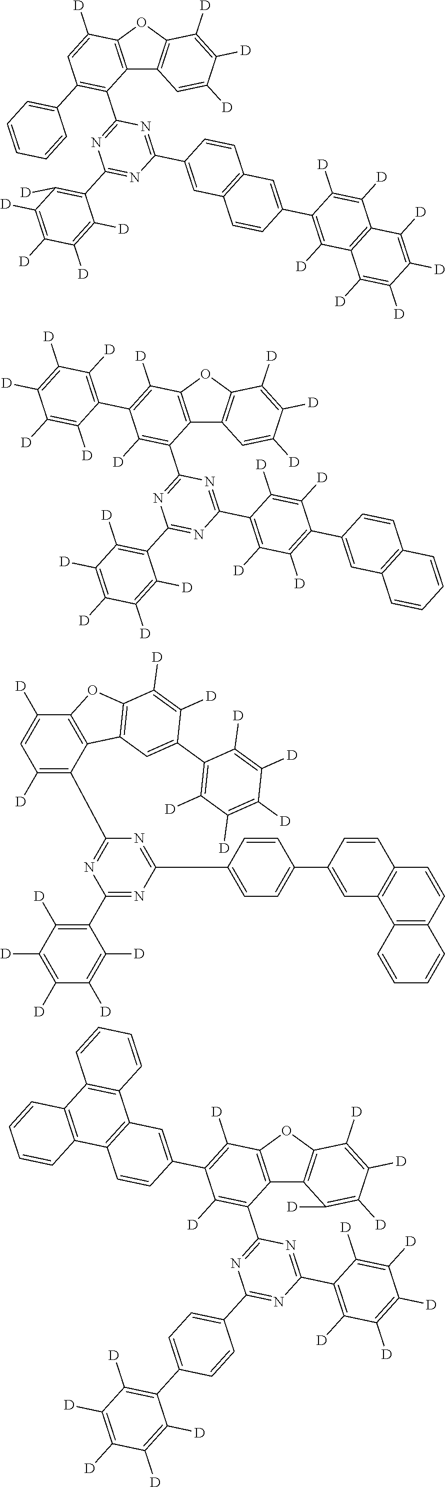

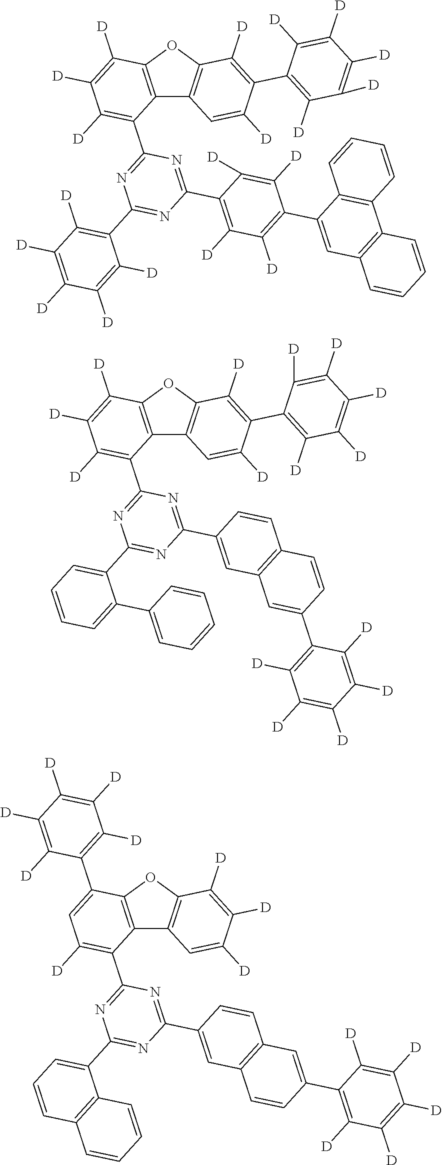

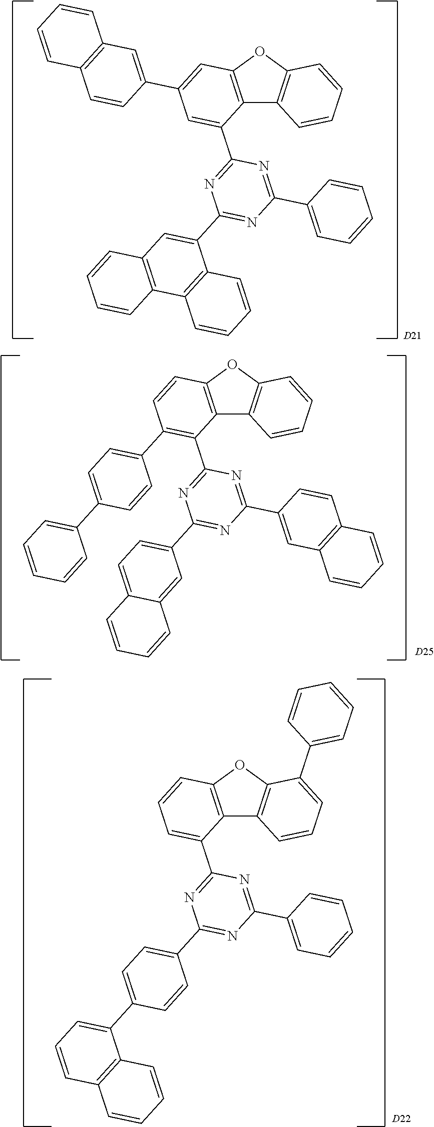

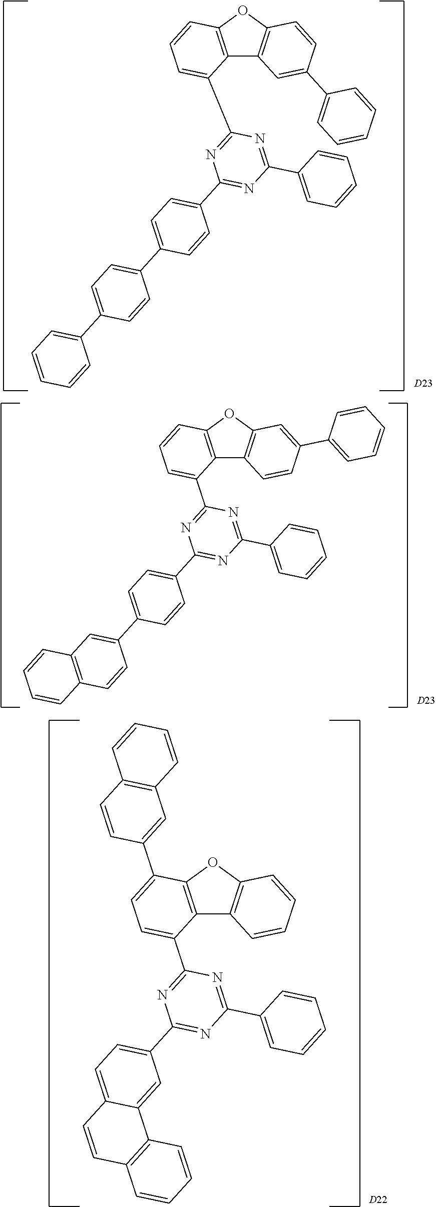

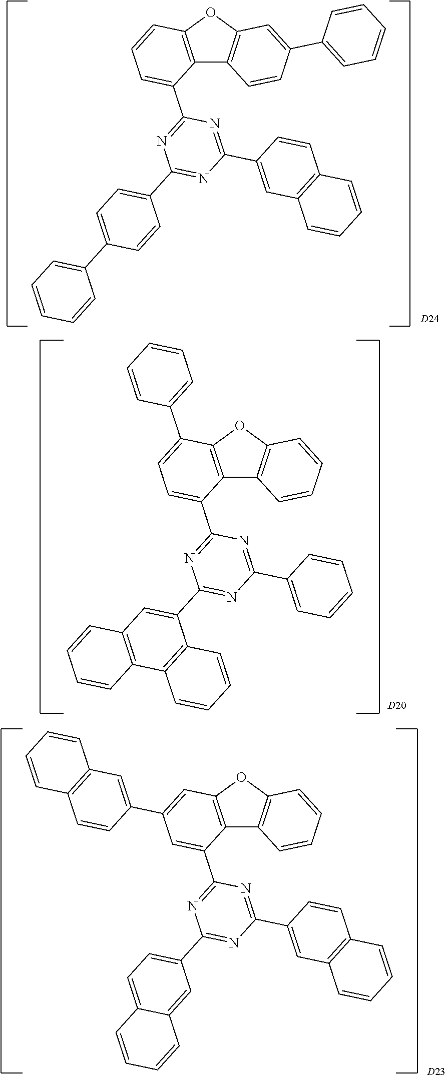

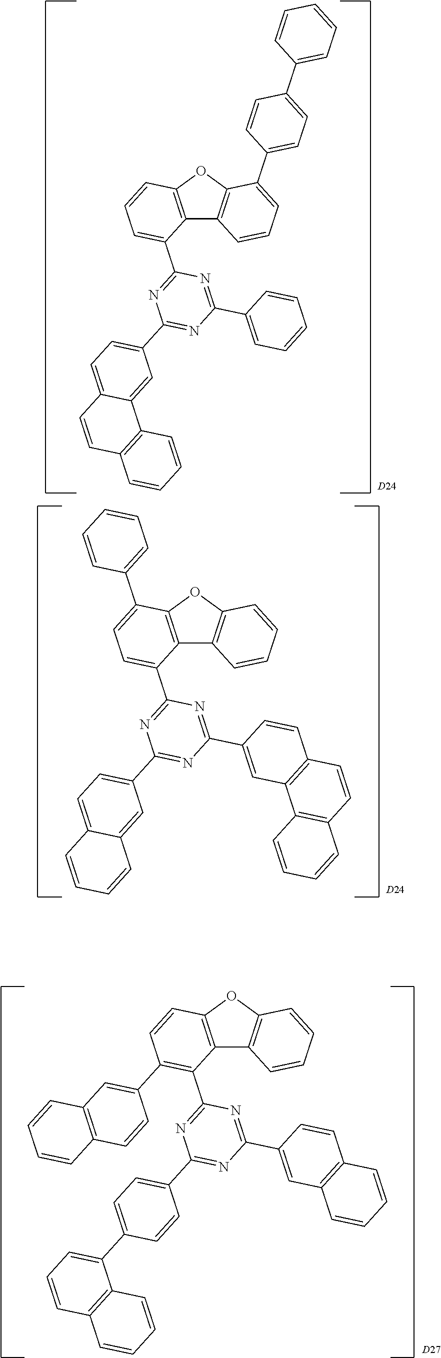

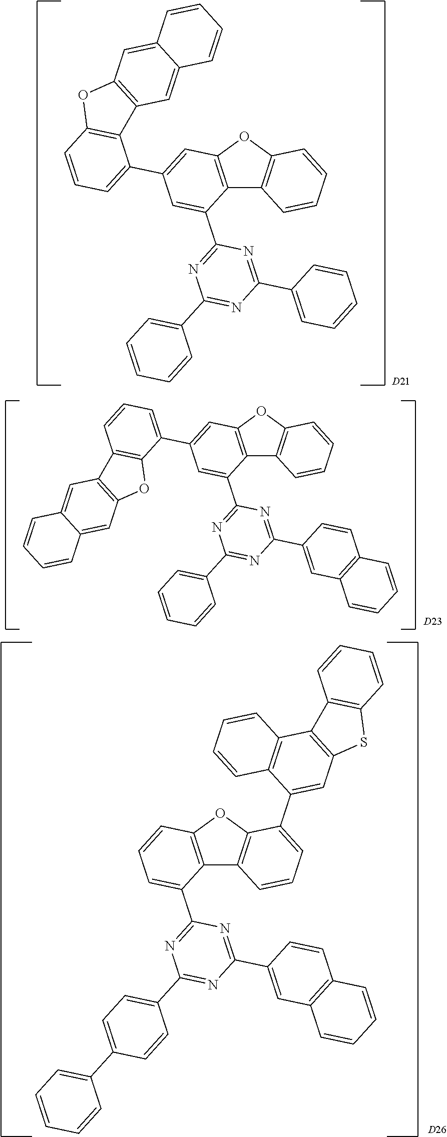

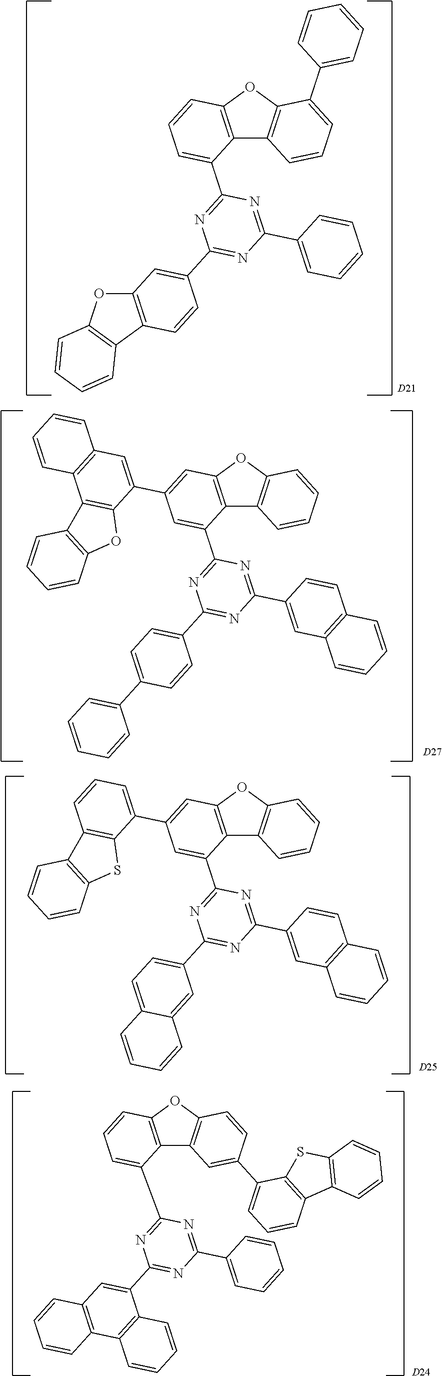

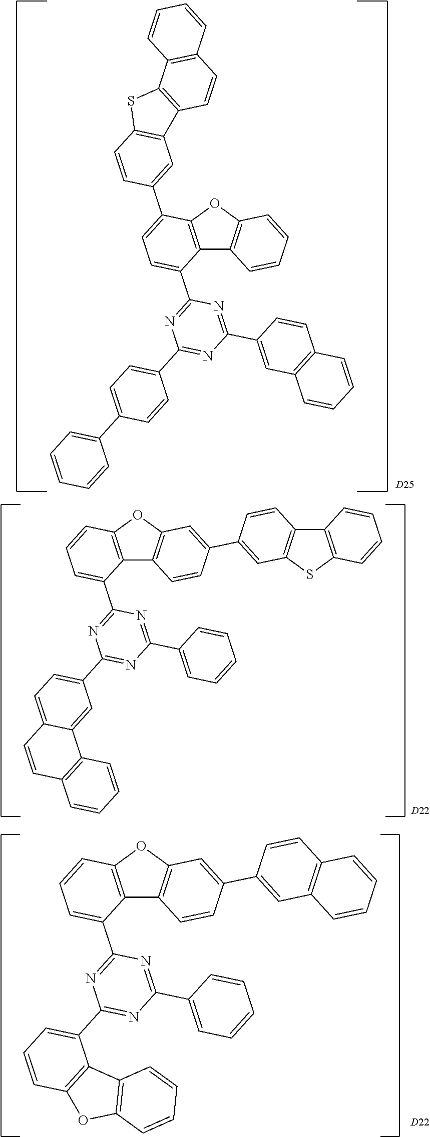

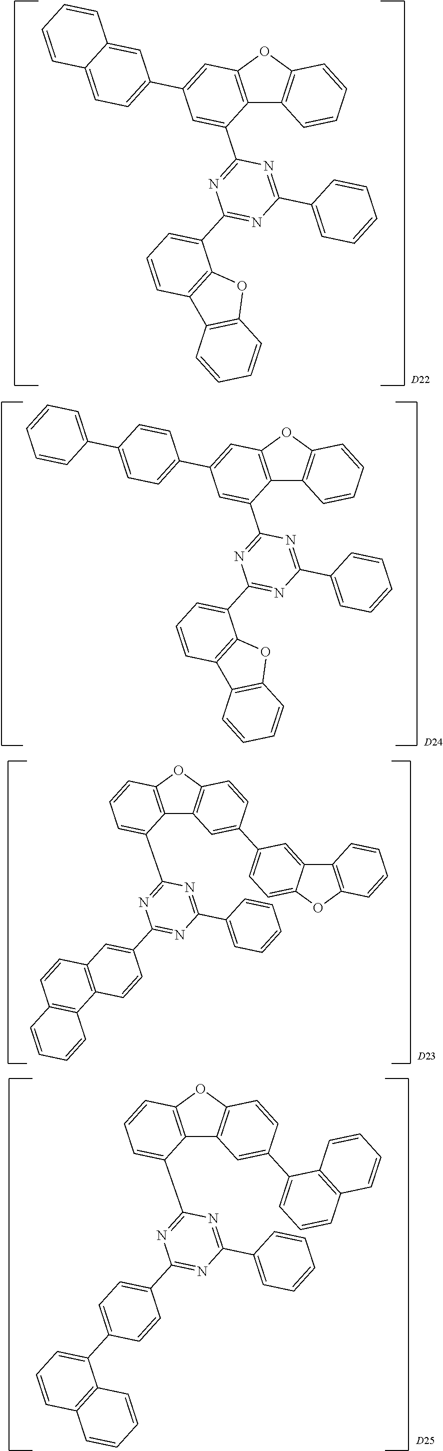

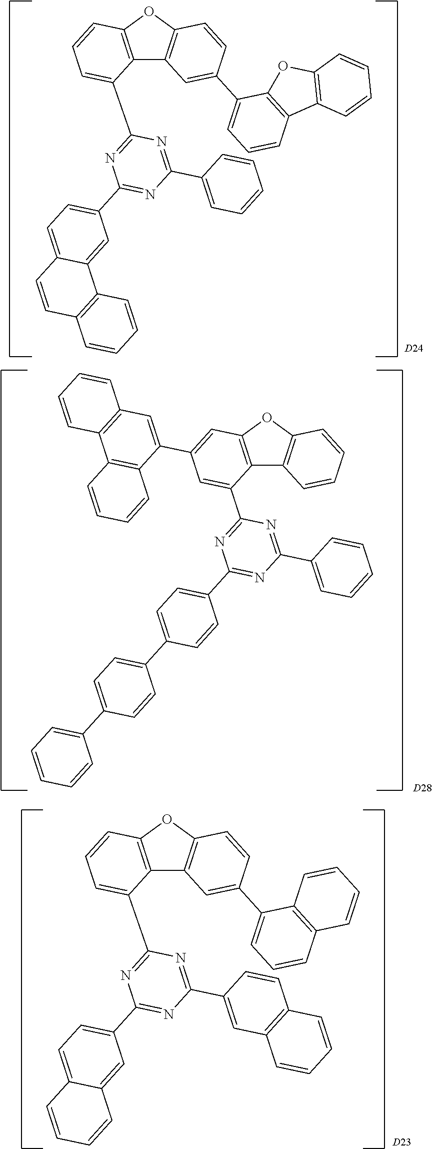

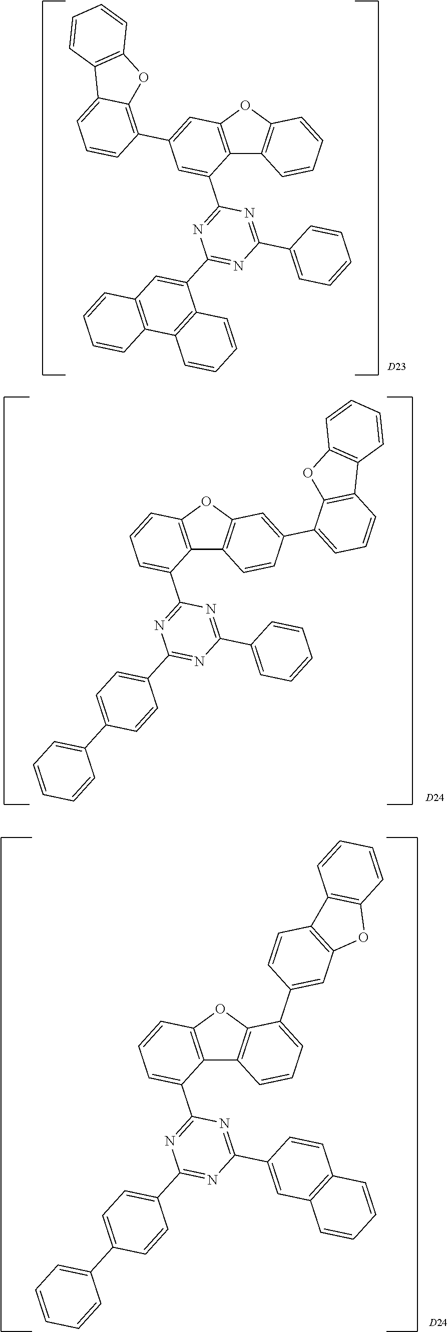

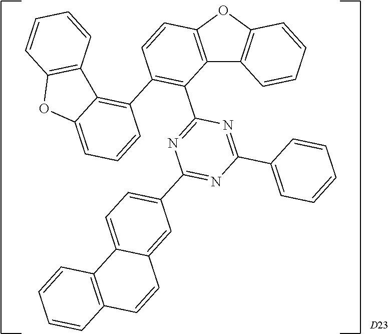

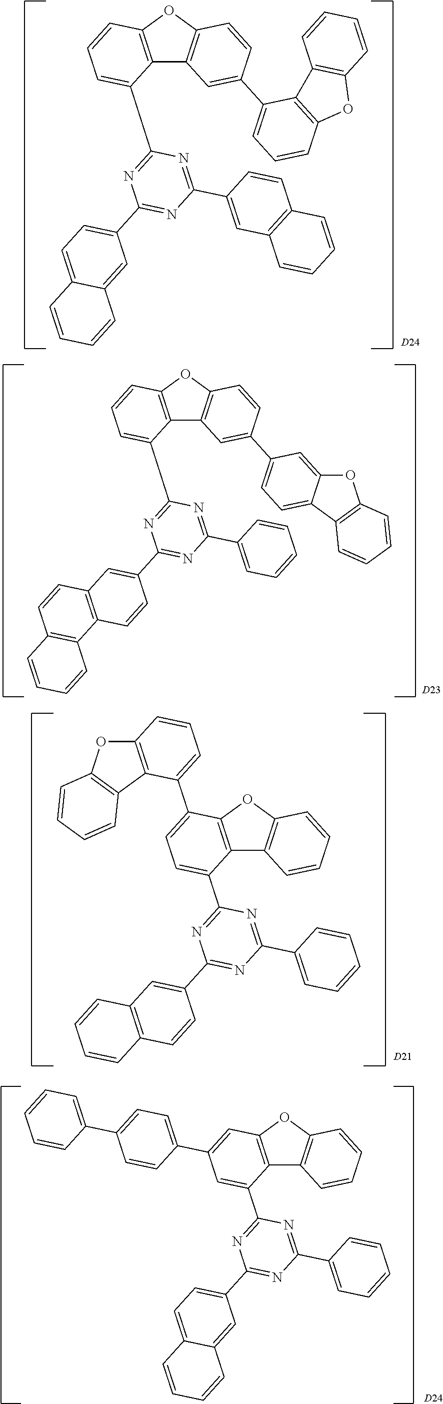

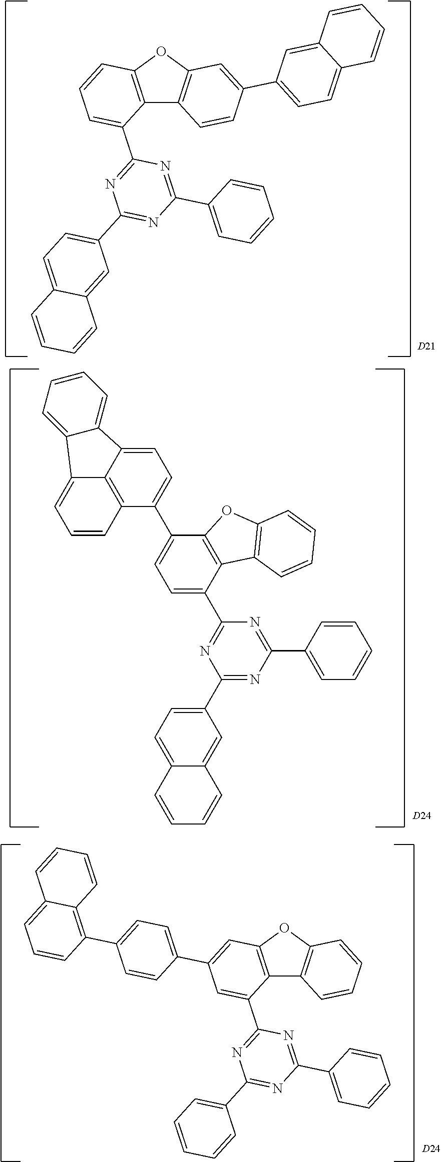

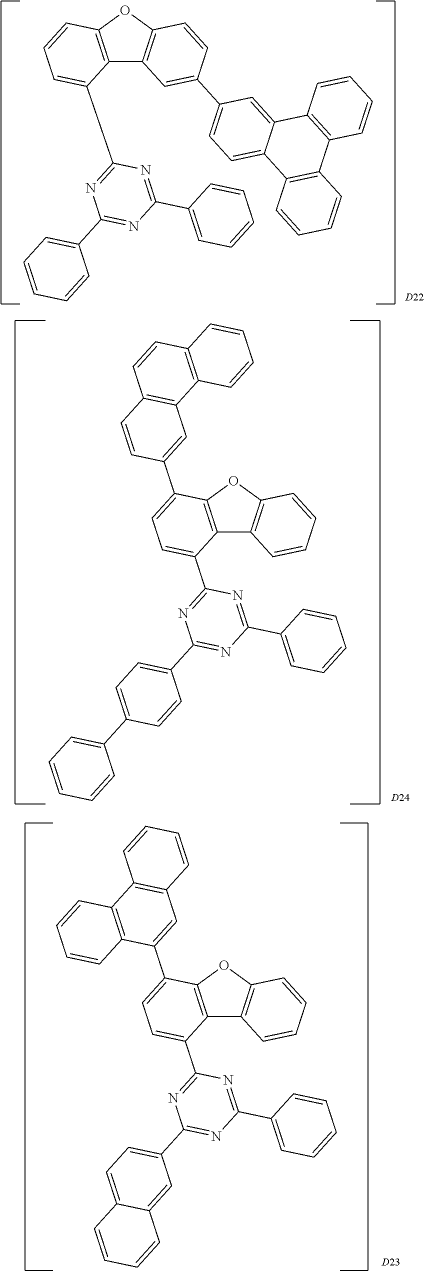

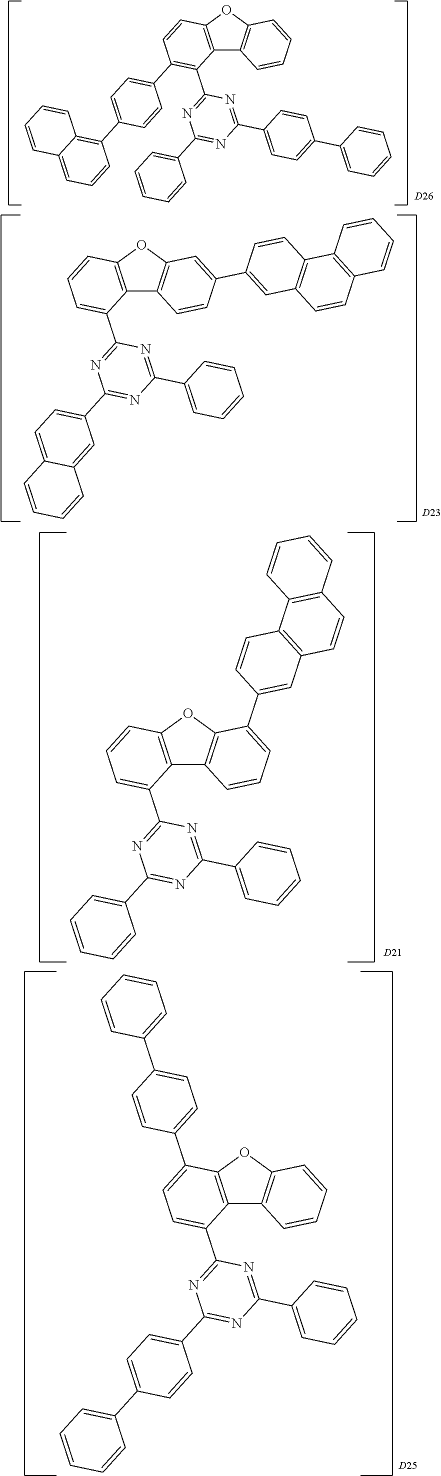

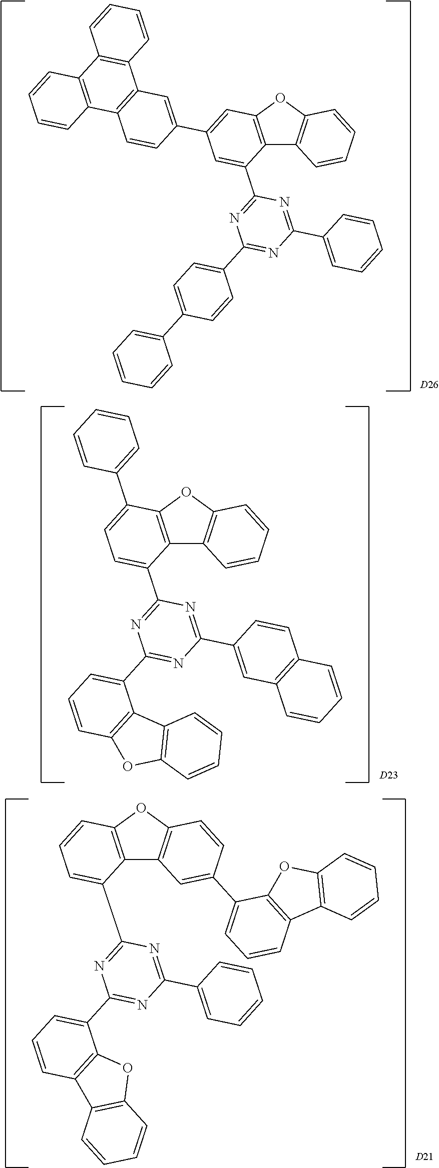

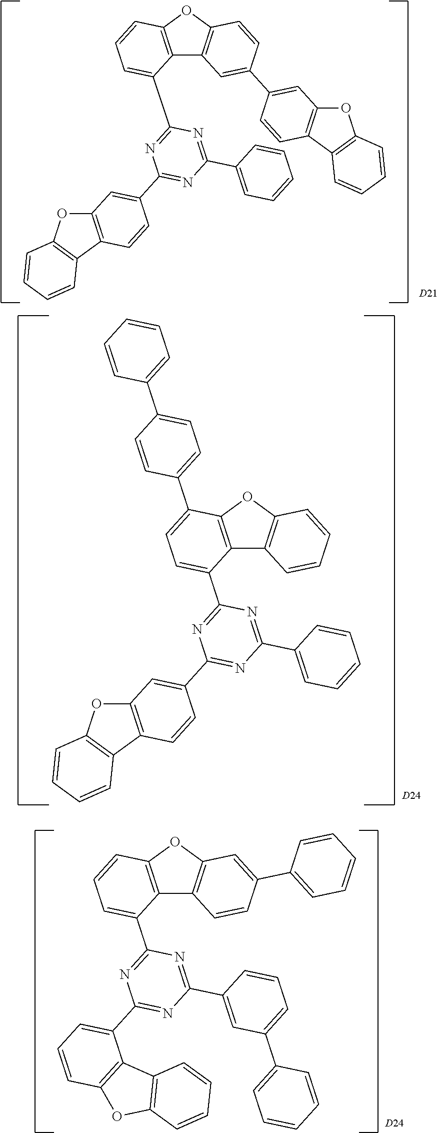

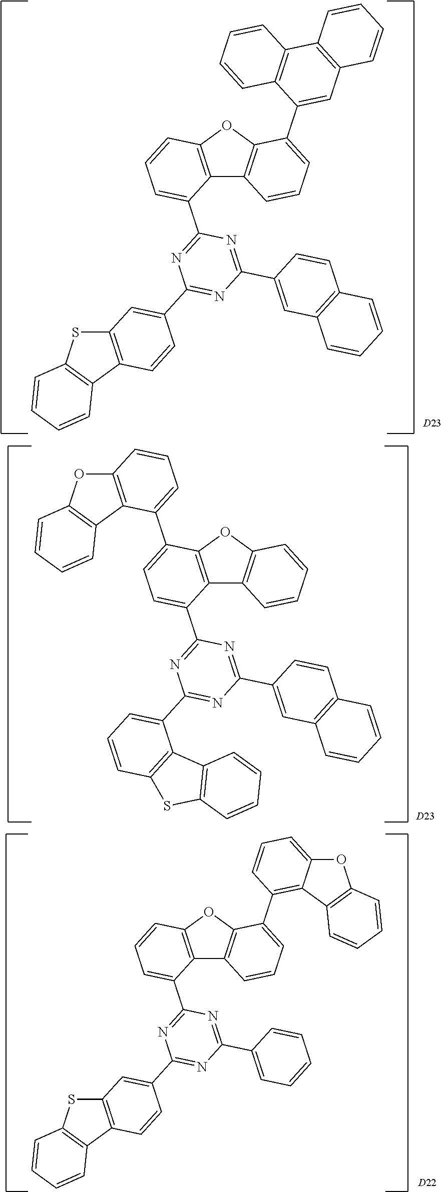

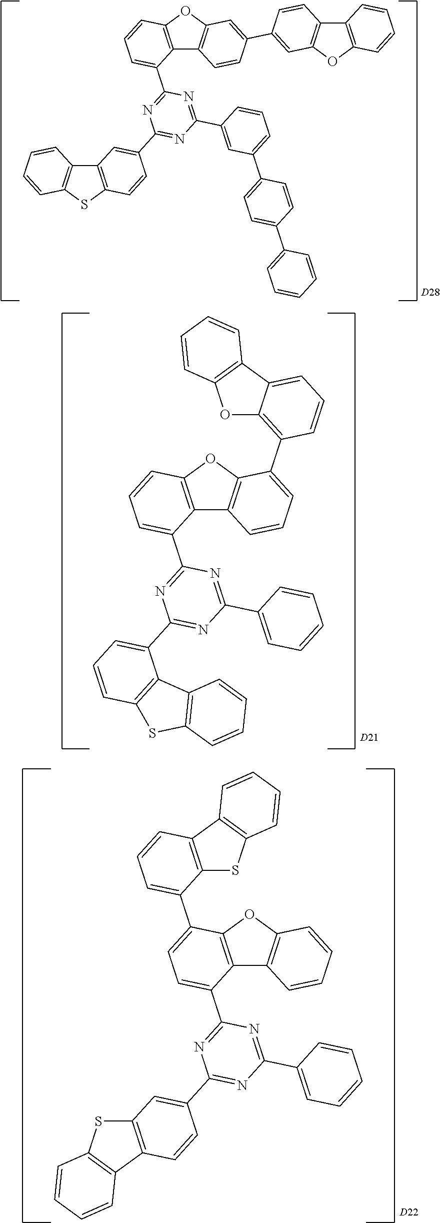

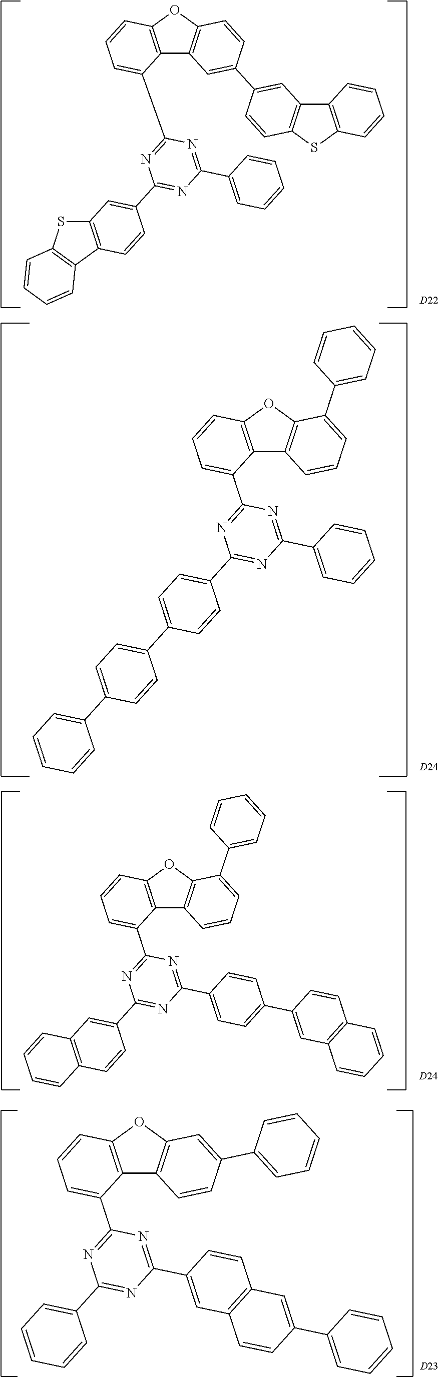

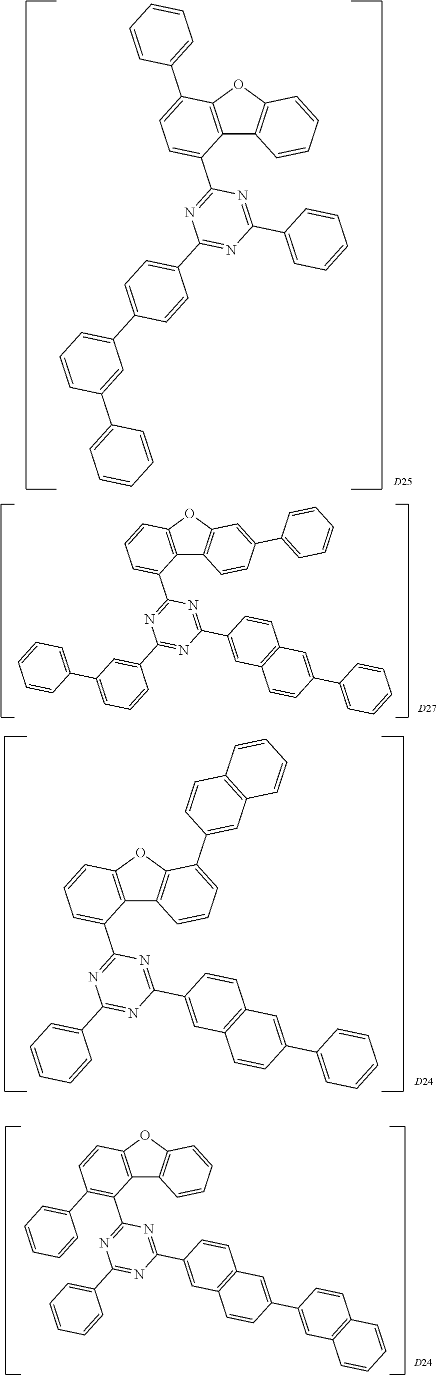

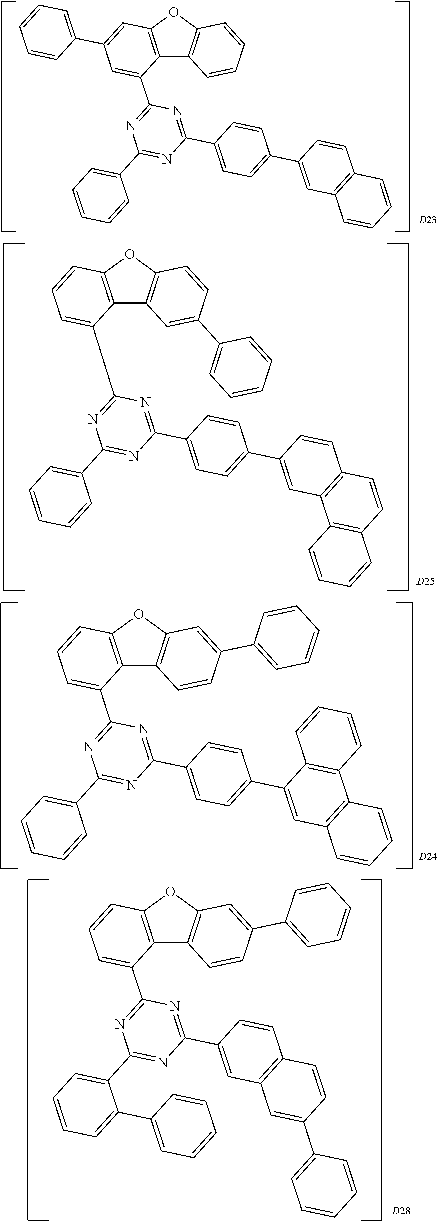

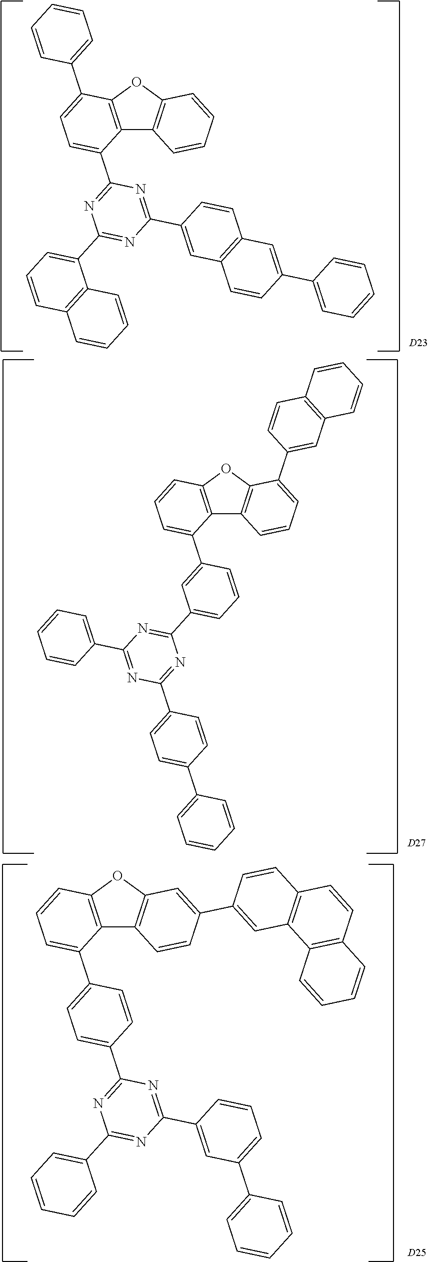

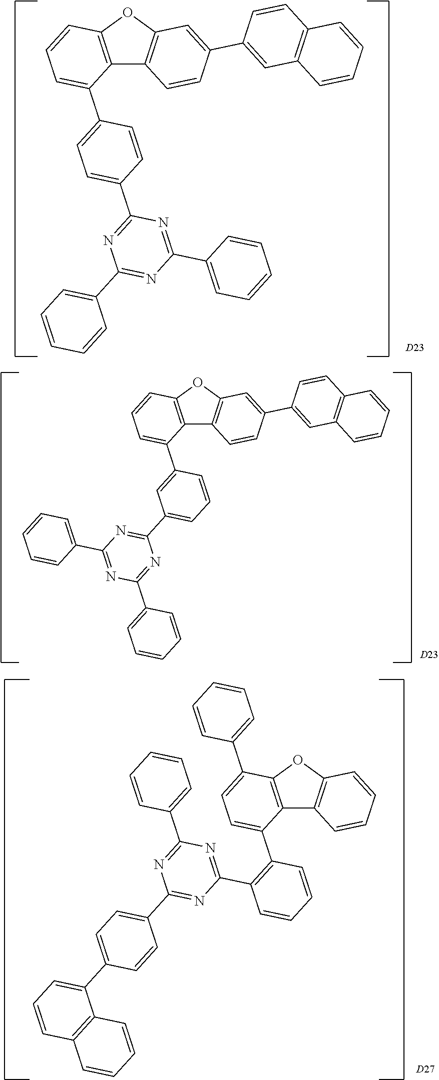

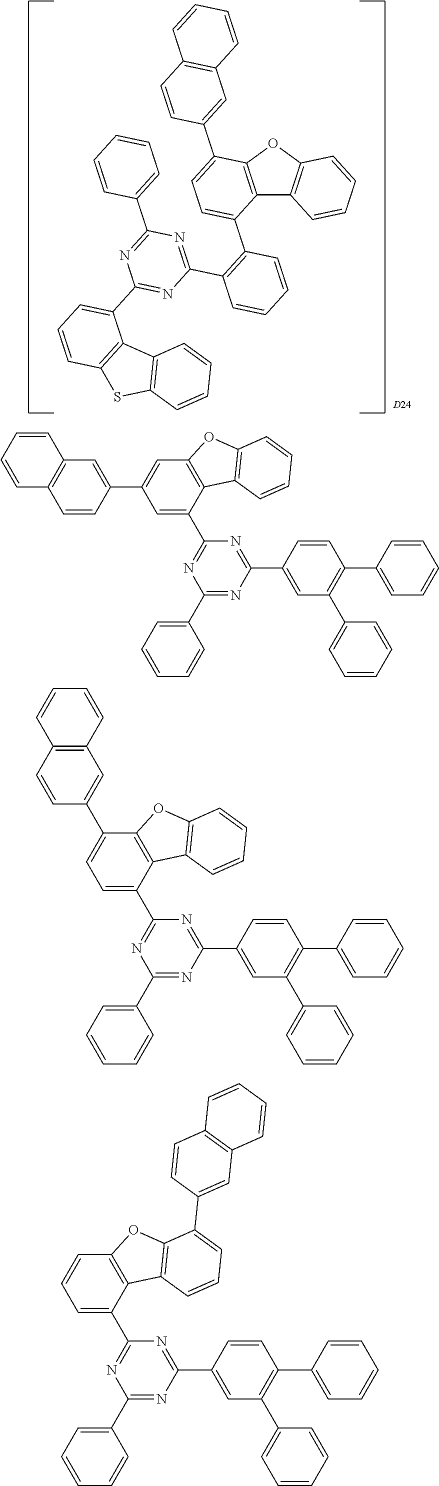

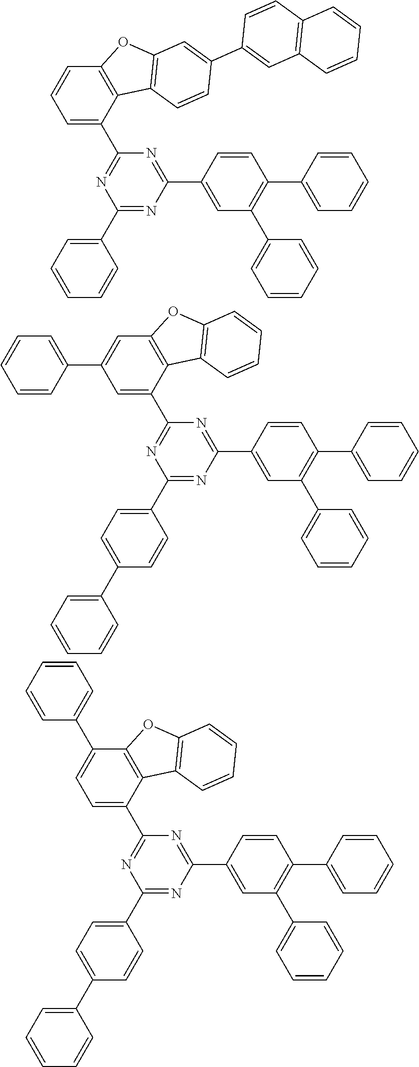

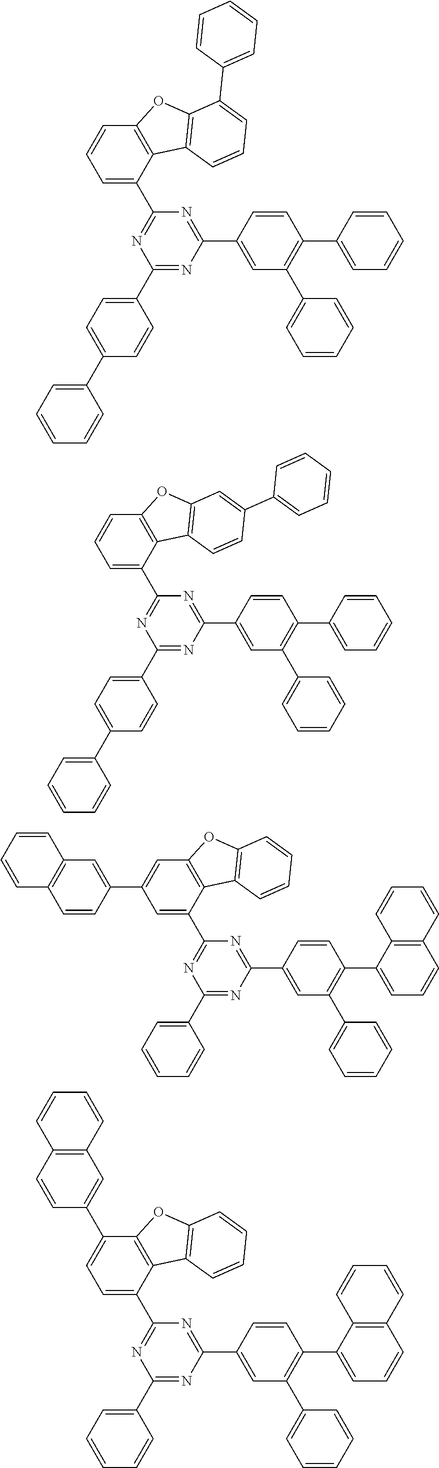

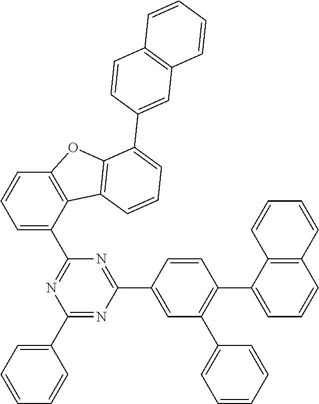 [SMILES: c1ccc(-c2nc(-c3ccc(-c4cccc5ccccc45)c(-c4ccccc4)c3)nc(-c3cccc4oc5c(-c6ccc7ccccc7c6)cccc5c34)n2)cc1]

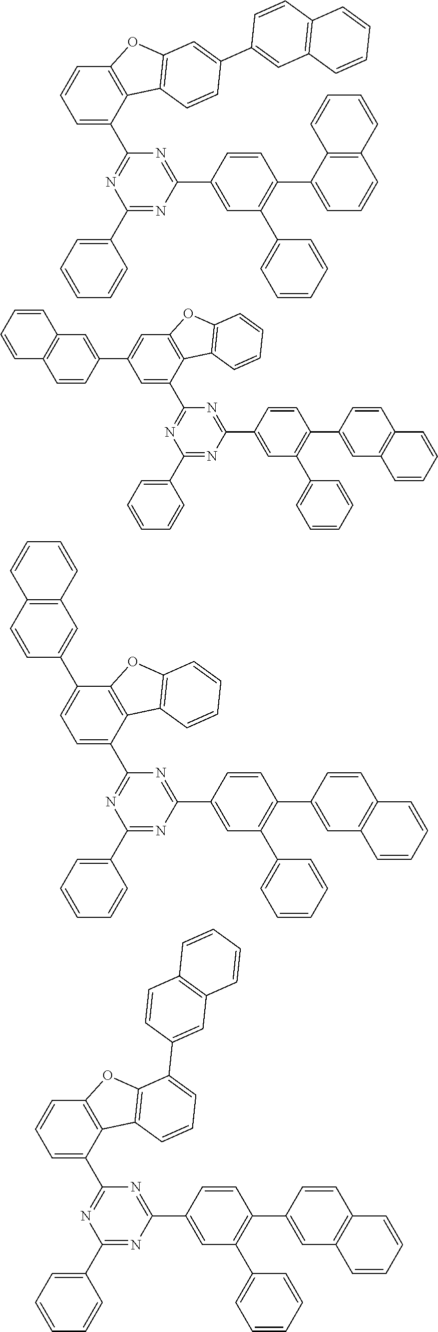

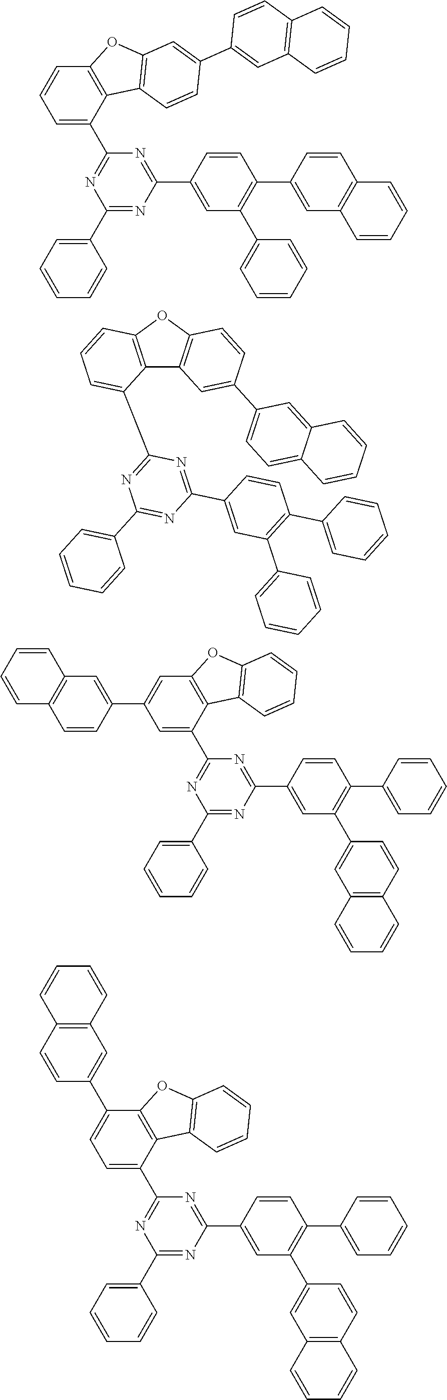

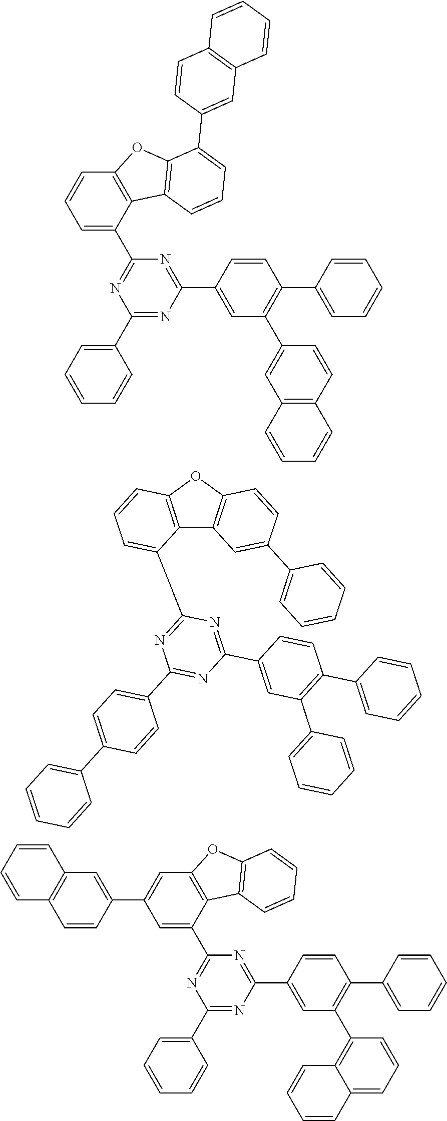

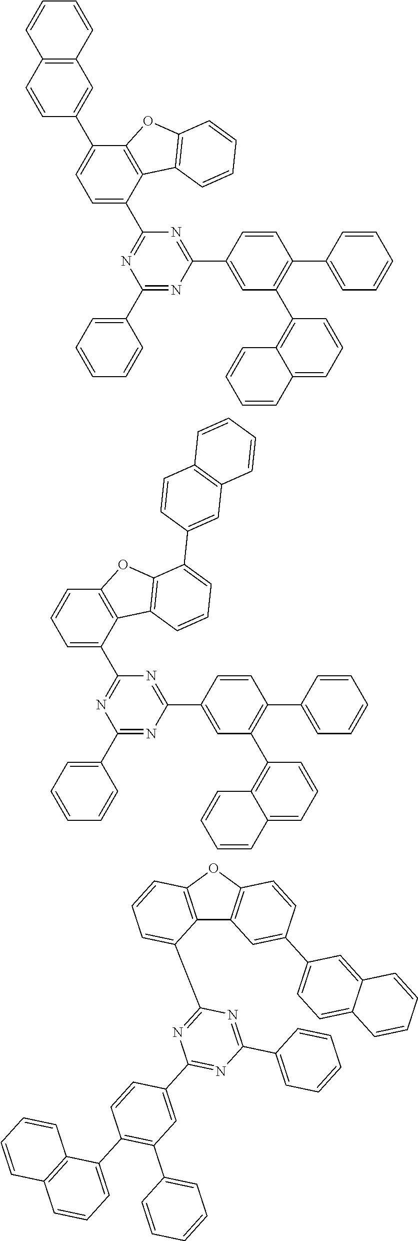

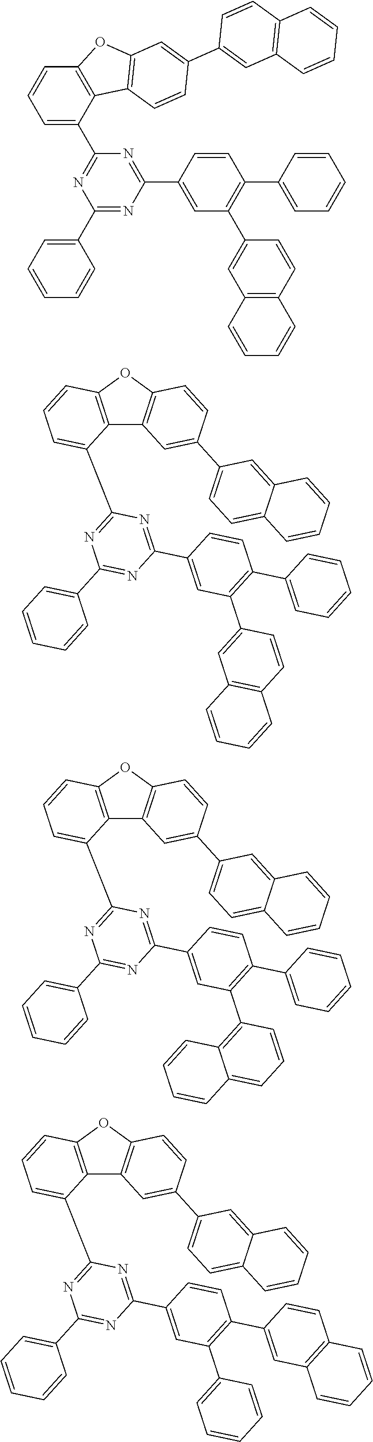

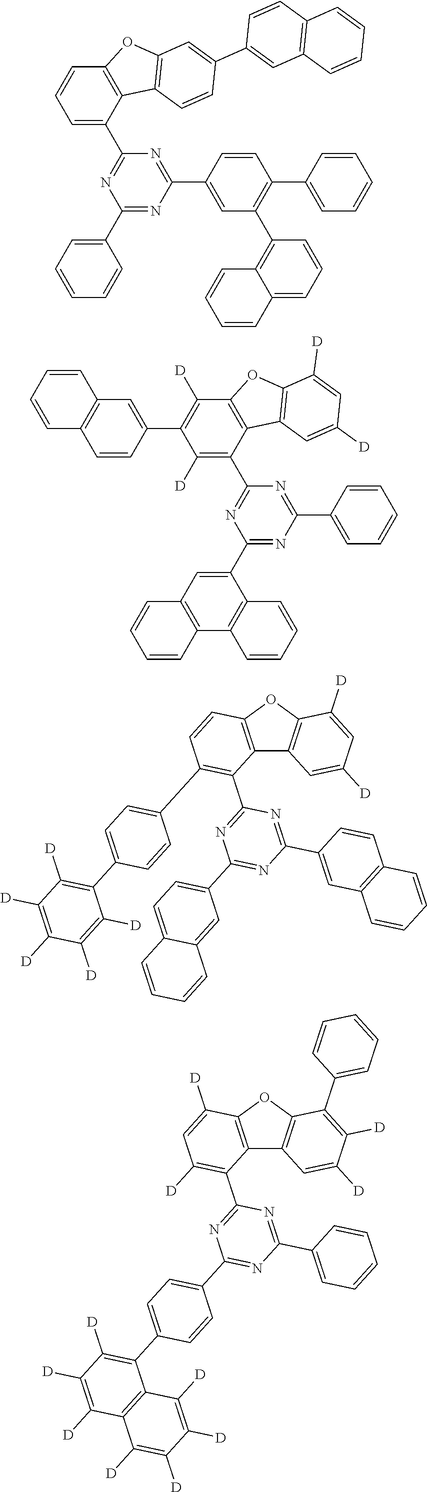

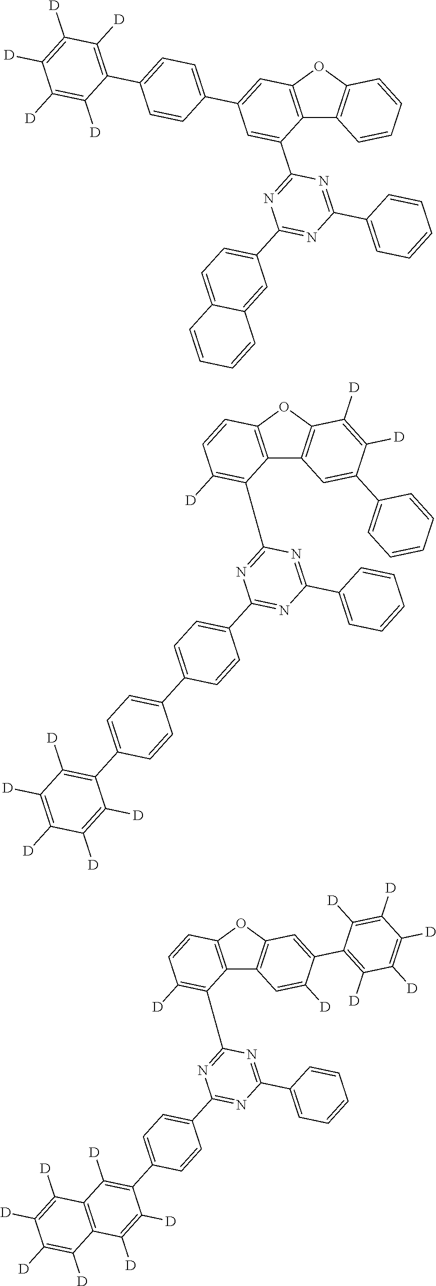 [SMILES: [2H]c1c([2H])c([2H])c(-c2ccc(-c3cc(-c4nc(-c5ccccc5)nc(-c5ccc6ccccc6c5)n4)c4c(c3)oc3ccccc34)cc2)c([2H])c1[2H] ; [2H]c1ccc2oc3c([2H])c([2H])c(-c4ccccc4)cc3c2c1-c1nc(-c2ccccc2)nc(-c2ccc(-c3ccc(-c4c([2H])c([2H])c([2H])c([2H])c4[2H])cc3)cc2)n1 ; [2H]c1cc2c(cc1-c1c([2H])c([2H])c([2H])c([2H])c1[2H])oc1ccc([2H])c(-c3nc(-c4ccccc4)nc(-c4ccc(-c5c([2H])c([2H])c6c([2H])c([2H])c([2H])c([2H])c6c5[2H])cc4)n3)c12]

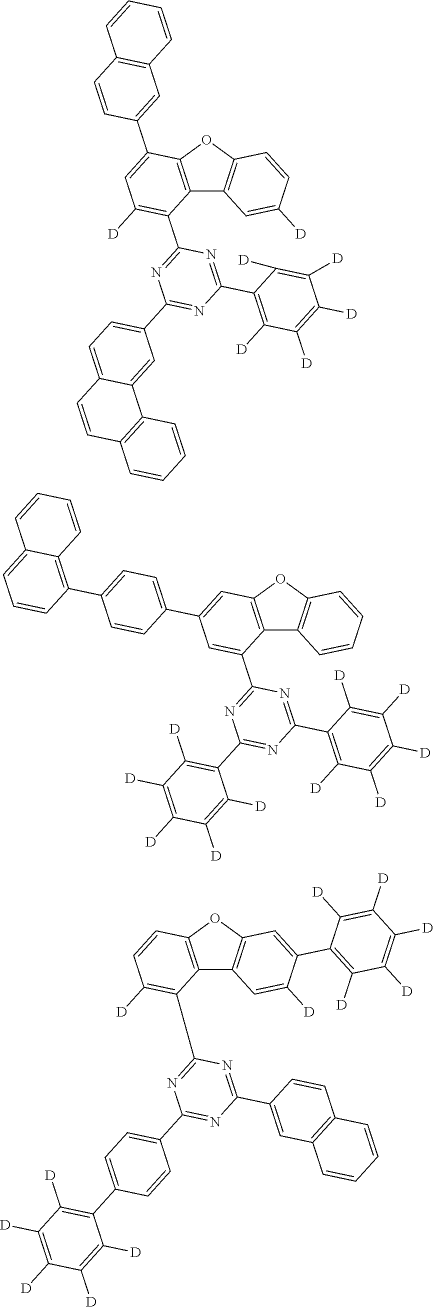 [SMILES: [2H]c1ccc2oc3c(-c4ccc5ccccc5c4)cc([2H])c(-c4nc(-c5ccc6ccc7ccccc7c6c5)nc(-c5c([2H])c([2H])c([2H])c([2H])c5[2H])n4)c3c2c1 ; [2H]c1c([2H])c([2H])c(-c2nc(-c3c([2H])c([2H])c([2H])c([2H])c3[2H])nc(-c3cc(-c4ccc(-c5cccc6ccccc56)cc4)cc4oc5ccccc5c34)n2)c([2H])c1[2H] ; [2H]c1cc2c(cc1-c1c([2H])c([2H])c([2H])c([2H])c1[2H])oc1ccc([2H])c(-c3nc(-c4ccc(-c5c([2H])c([2H])c([2H])c([2H])c5[2H])cc4)nc(-c4ccc5ccccc5c4)n3)c12]

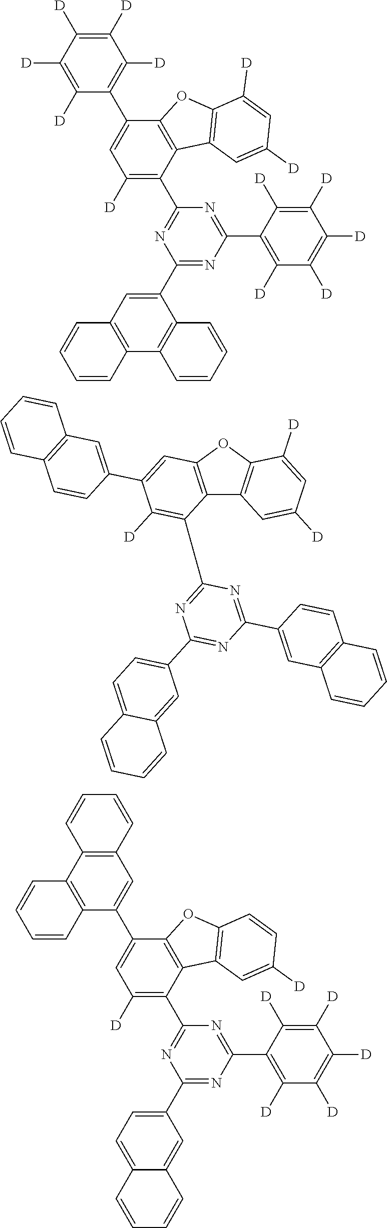

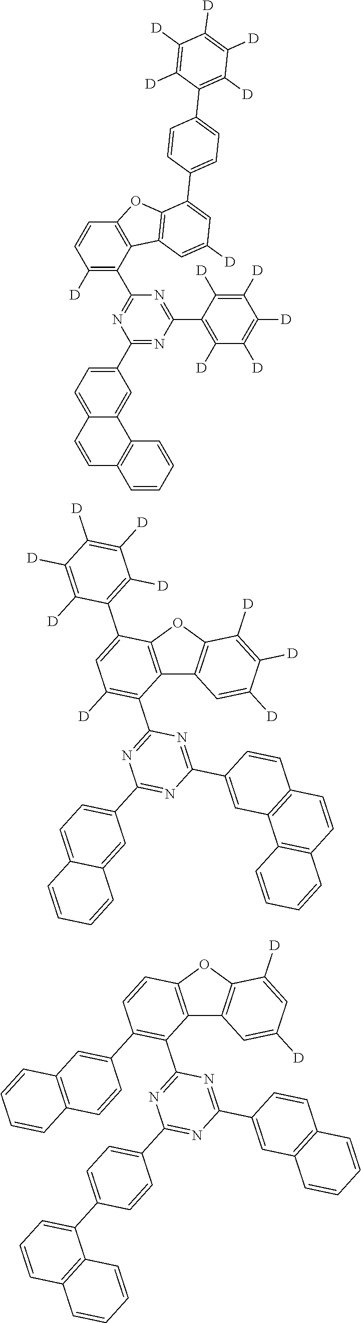

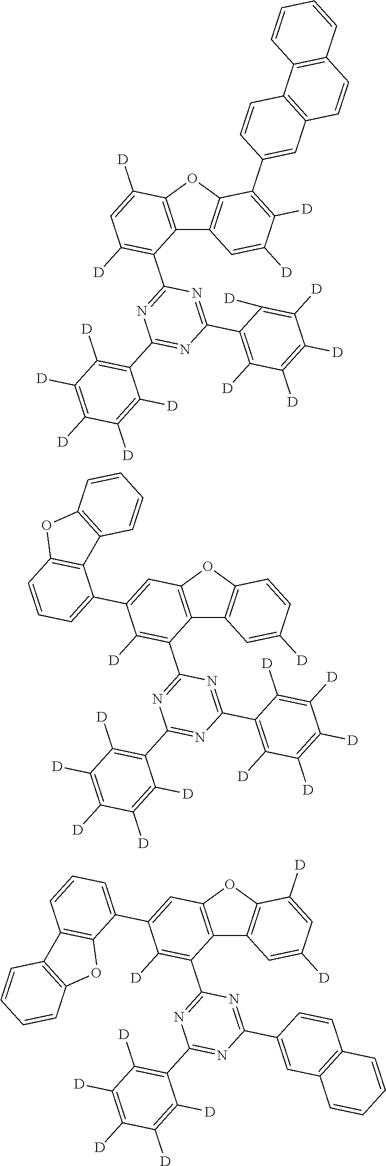

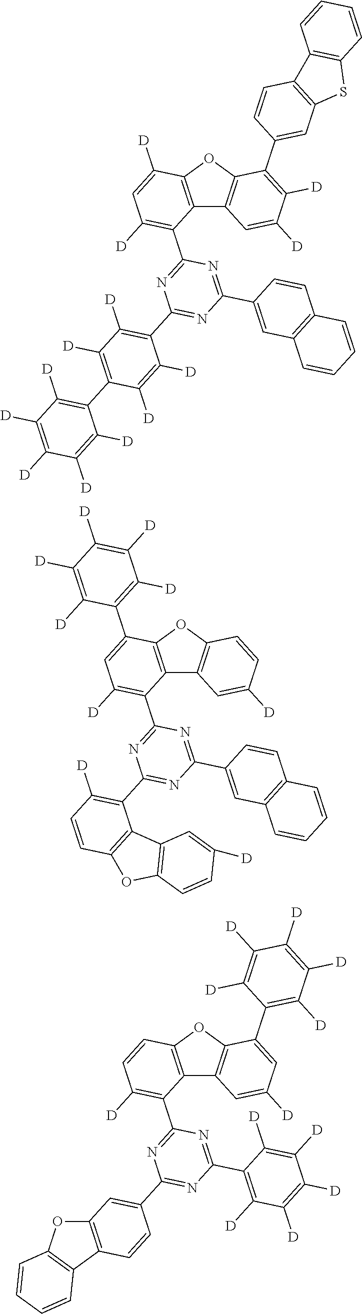

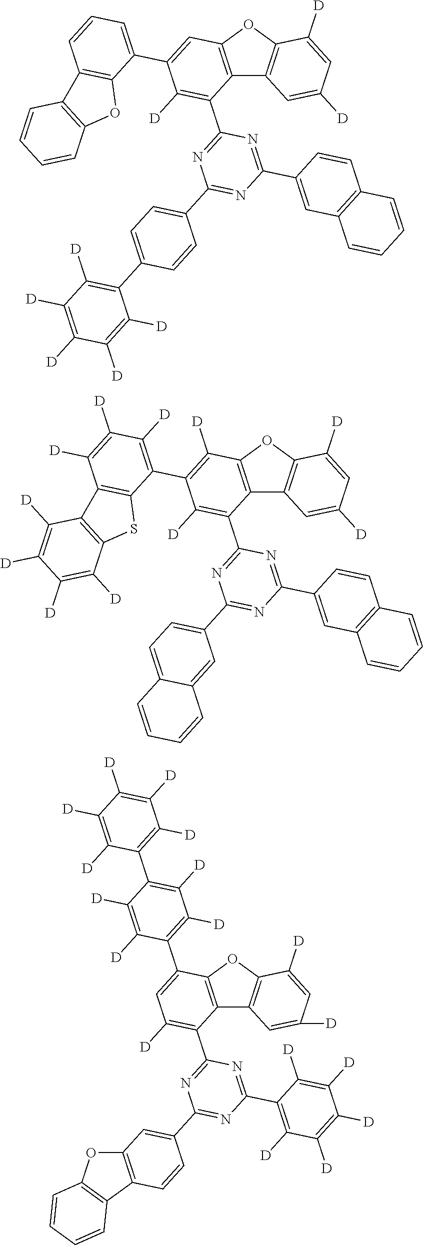

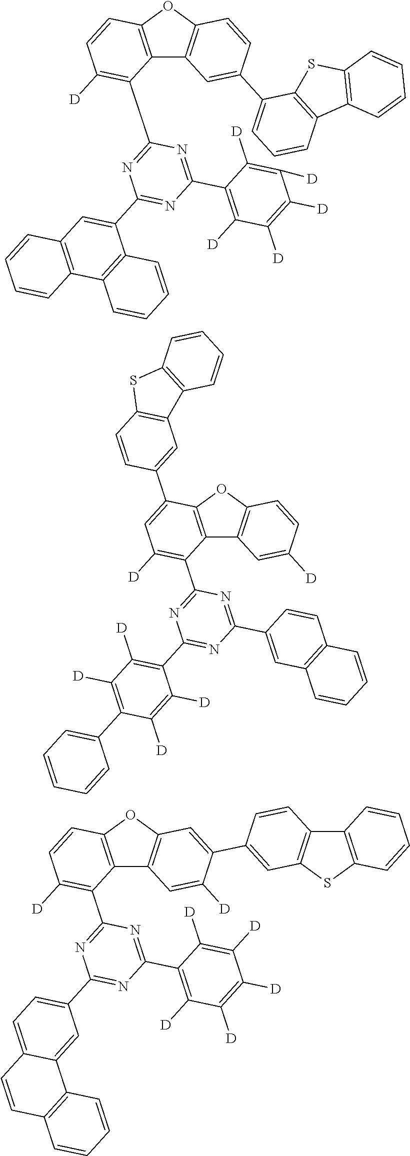

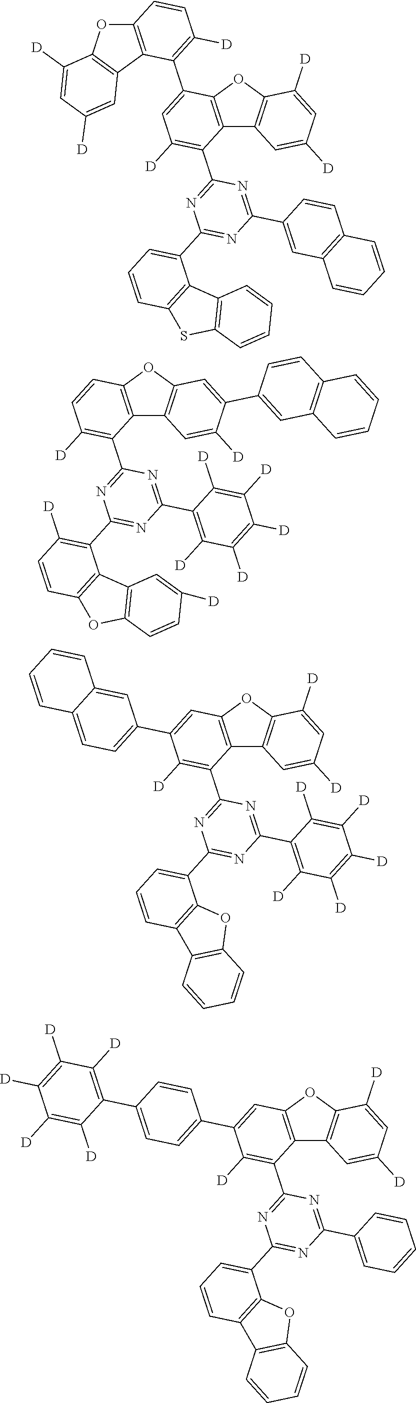

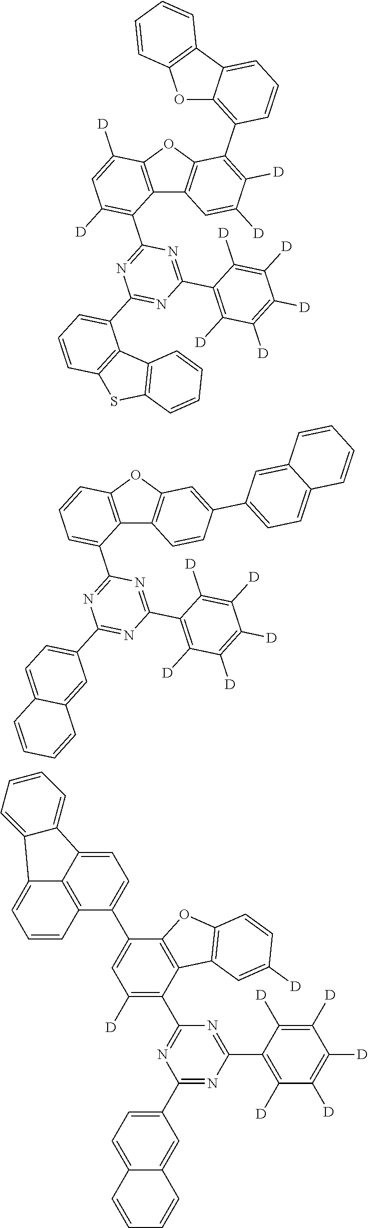

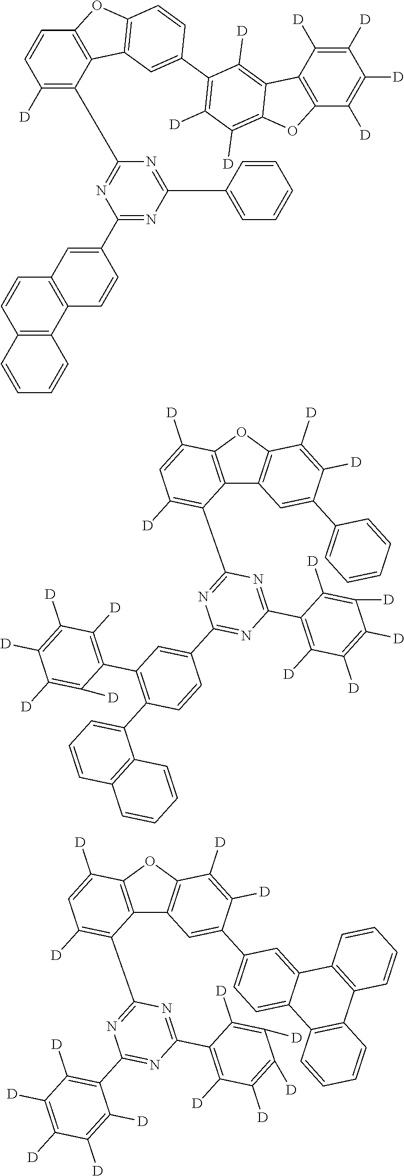

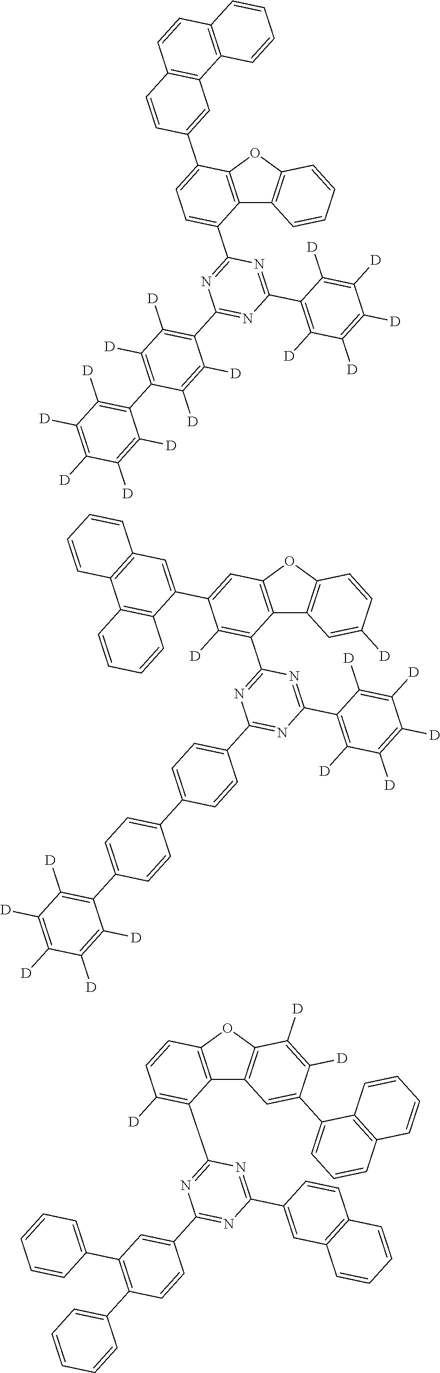

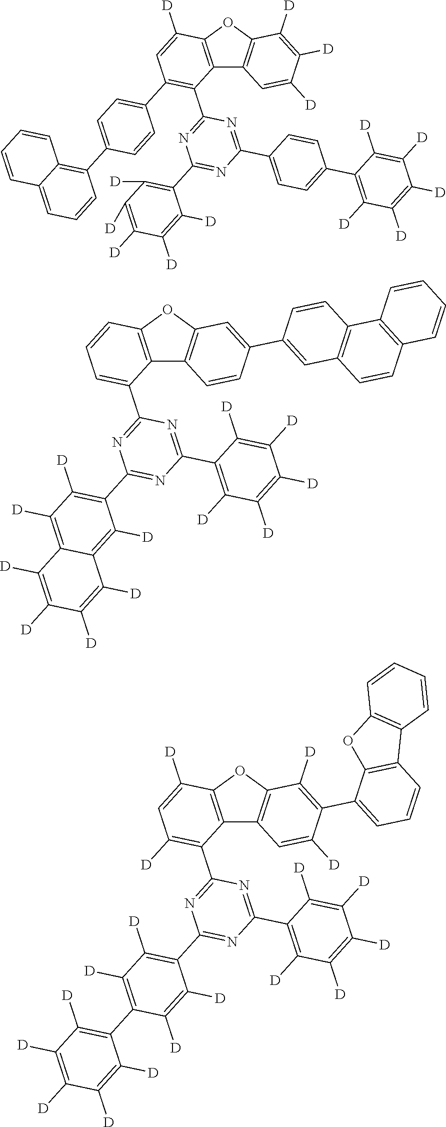

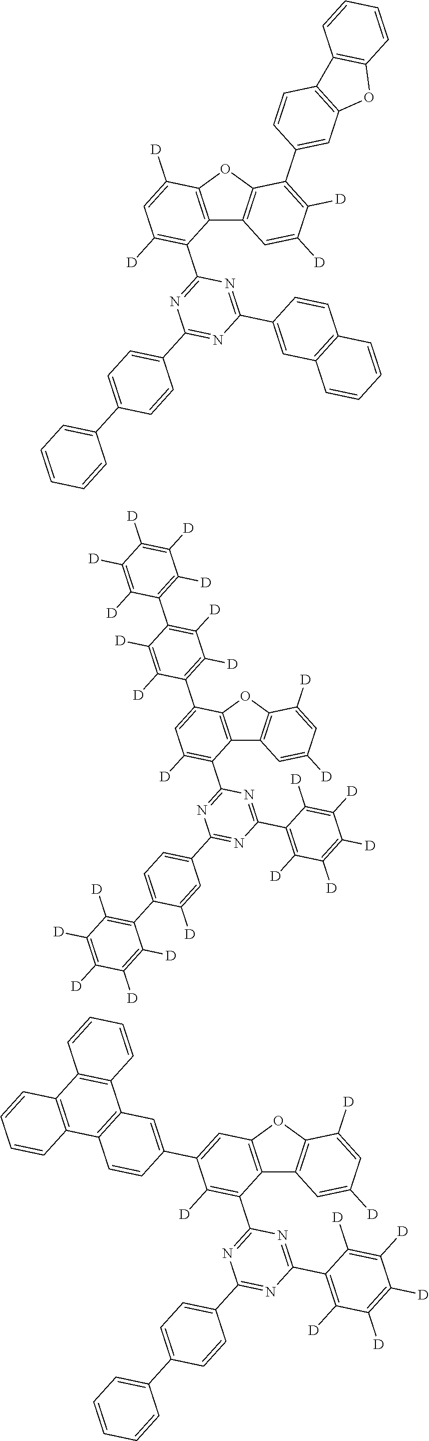

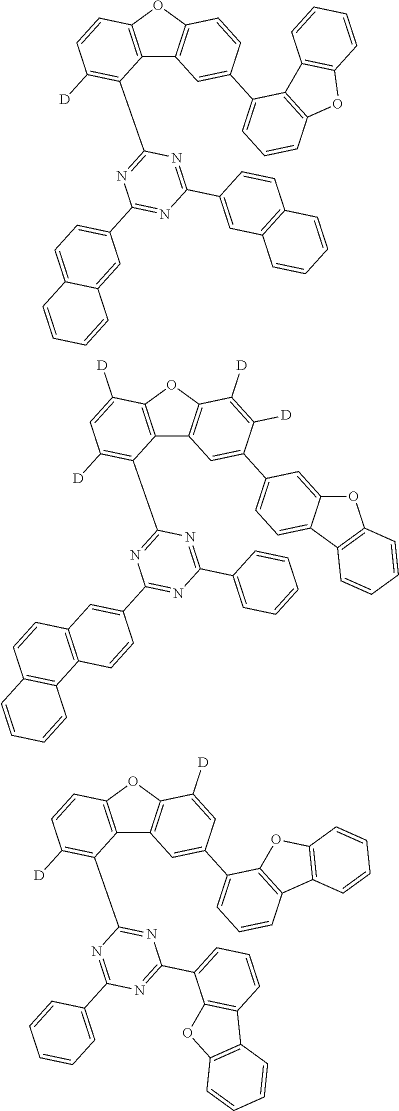

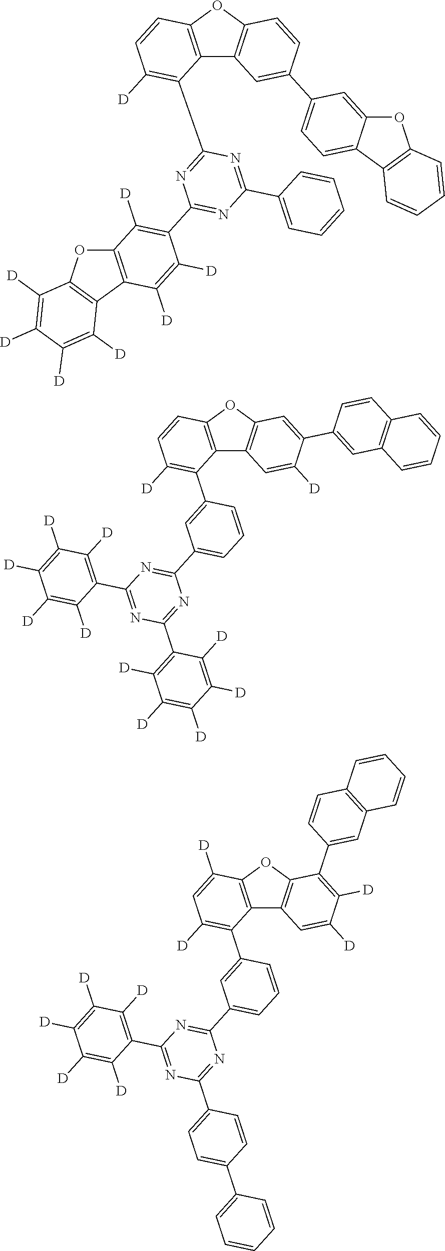 [SMILES: [2H]c1ccc2oc3ccc(-c4ccc5c(c4)oc4ccccc45)cc3c2c1-c1nc(-c2ccccc2)nc(-c2c([2H])c([2H])c3c(oc4c([2H])c([2H])c([2H])c([2H])c43)c2[2H])n1 ; [2H]c1cc2c(cc1-c1ccc3ccccc3c1)oc1ccc([2H])c(-c3cccc(-c4nc(-c5c([2H])c([2H])c([2H])c([2H])c5[2H])nc(-c5c([2H])c([2H])c([2H])c([2H])c5[2H])n4)c3)c12 ; [2H]c1cc2c(oc3c([2H])cc([2H])c(-c4cccc(-c5nc(-c6ccc(-c7ccccc7)cc6)nc(-c6c([2H])c([2H])c([2H])c([2H])c6[2H])n5)c4)c32)c(-c2ccc3ccccc3c2)c1[2H]]

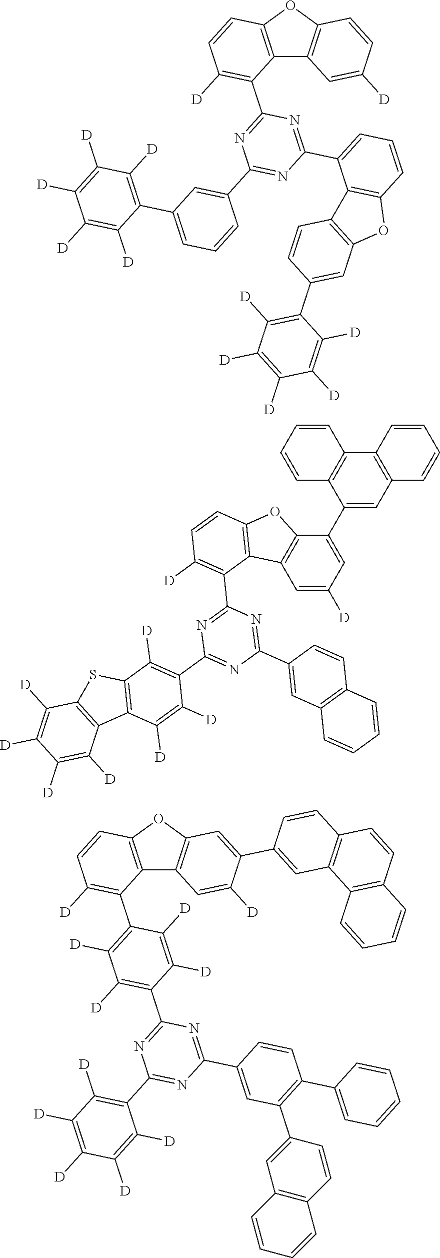

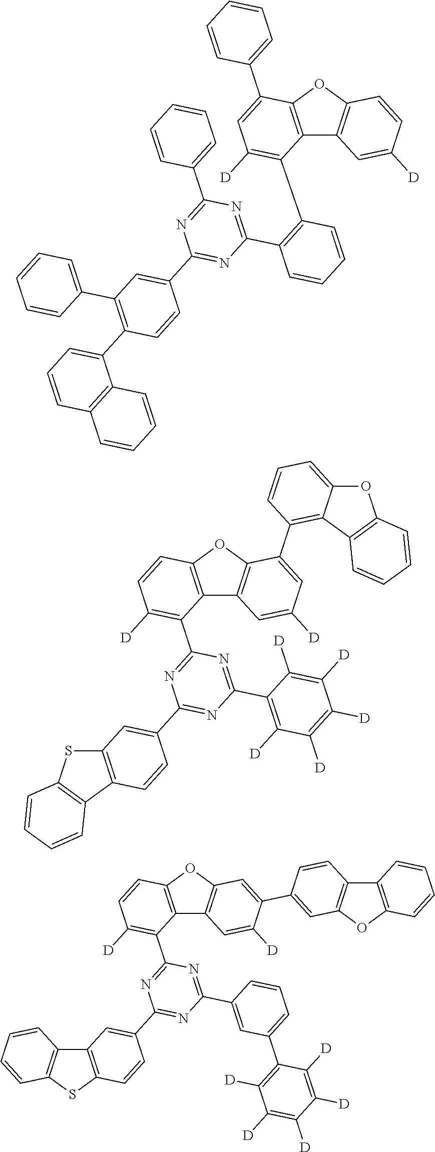 [SMILES: [2H]c1ccc2oc3c(-c4ccccc4)cc([2H])c(-c4ccccc4-c4nc(-c5ccccc5)nc(-c5ccc(-c6cccc7ccccc67)c(-c6ccccc6)c5)n4)c3c2c1 ; [2H]c1cc(-c2cccc3oc4ccccc4c23)c2oc3ccc([2H])c(-c4nc(-c5ccc6c(c5)sc5ccccc56)nc(-c5c([2H])c([2H])c([2H])c([2H])c5[2H])n4)c3c2c1 ; [2H]c1cc2c(cc1-c1ccc3c(c1)oc1ccccc13)oc1ccc([2H])c(-c3nc(-c4cccc(-c5c([2H])c([2H])c([2H])c([2H])c5[2H])c4)nc(-c4ccc5sc6ccccc6c5c4)n3)c12]

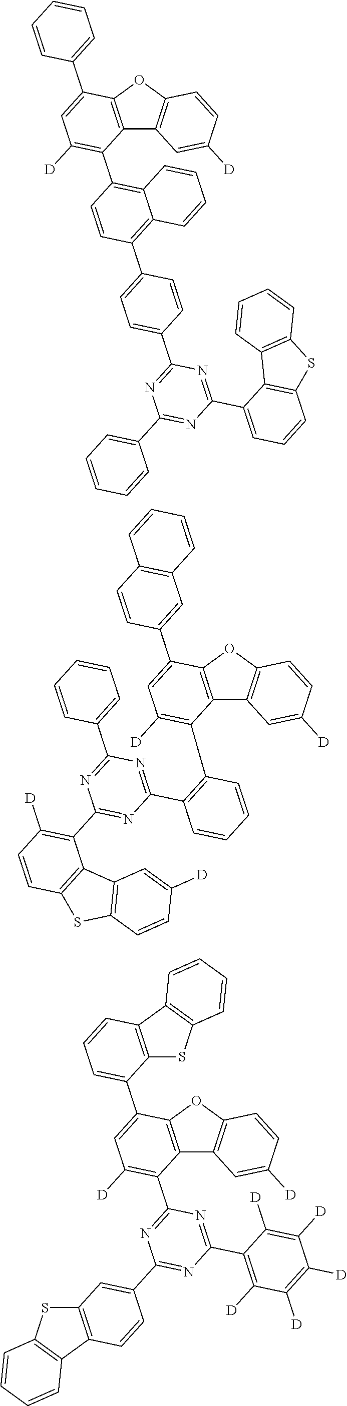

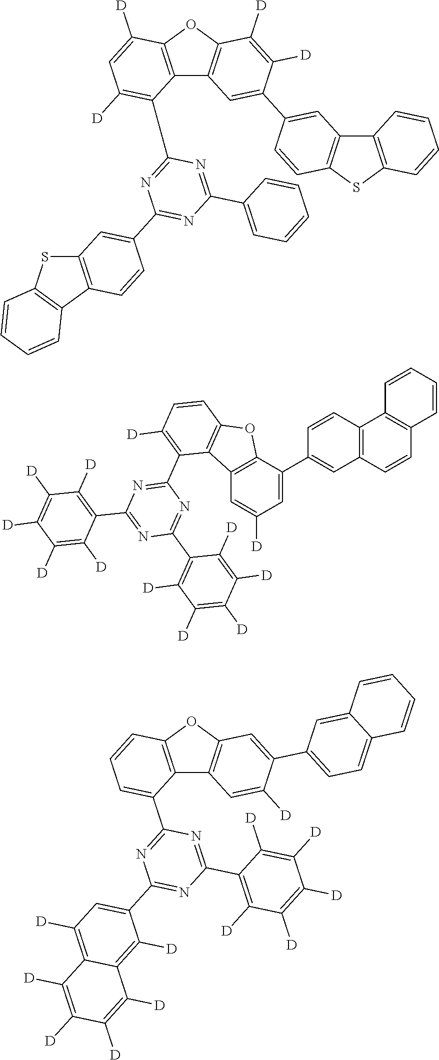 [SMILES: [2H]c1cc([2H])c2oc3c([2H])c([2H])c(-c4ccc5sc6ccccc6c5c4)cc3c2c1-c1nc(-c2ccccc2)nc(-c2ccc3c(c2)sc2ccccc23)n1 ; [2H]c1cc(-c2ccc3c(ccc4ccccc43)c2)c2oc3ccc([2H])c(-c4nc(-c5c([2H])c([2H])c([2H])c([2H])c5[2H])nc(-c5c([2H])c([2H])c([2H])c([2H])c5[2H])n4)c3c2c1 ; [2H]c1cc2c(cc1-c1ccc3ccccc3c1)oc1cccc(-c3nc(-c4cc([2H])c5c([2H])c([2H])c([2H])c([2H])c5c4[2H])nc(-c4c([2H])c([2H])c([2H])c([2H])c4[2H])n3)c12]

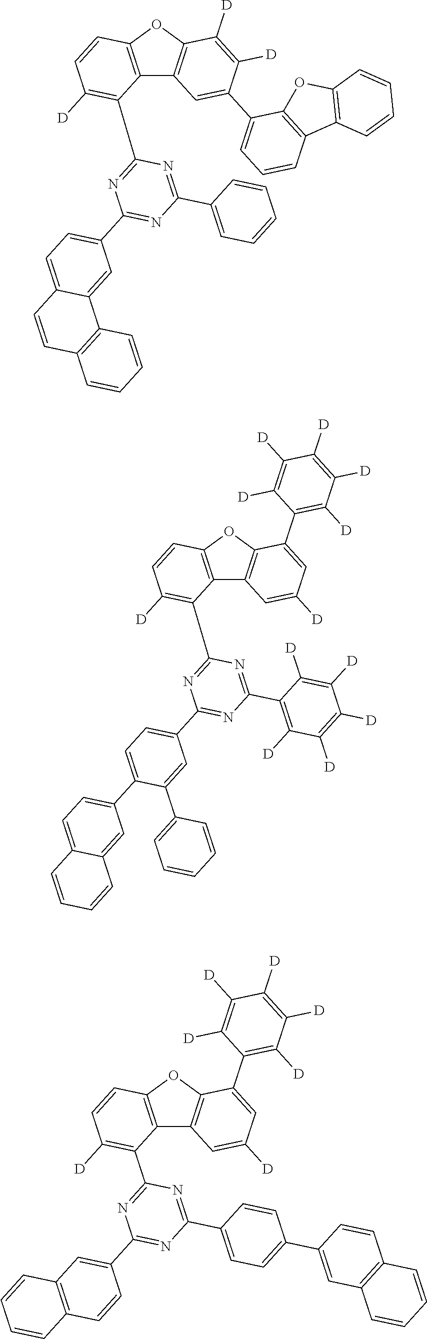 [SMILES: [2H]c1ccc2oc3c([2H])c([2H])c(-c4cccc5c4oc4ccccc45)cc3c2c1-c1nc(-c2ccccc2)nc(-c2ccc3ccc4ccccc4c3c2)n1 ; [2H]c1cc(-c2c([2H])c([2H])c([2H])c([2H])c2[2H])c2oc3ccc([2H])c(-c4nc(-c5ccc(-c6ccc7ccccc7c6)c(-c6ccccc6)c5)nc(-c5c([2H])c([2H])c([2H])c([2H])c5[2H])n4)c3c2c1 ; [2H]c1cc(-c2c([2H])c([2H])c([2H])c([2H])c2[2H])c2oc3ccc([2H])c(-c4nc(-c5ccc(-c6ccc7ccccc7c6)cc5)nc(-c5ccc6ccccc6c5)n4)c3c2c1]

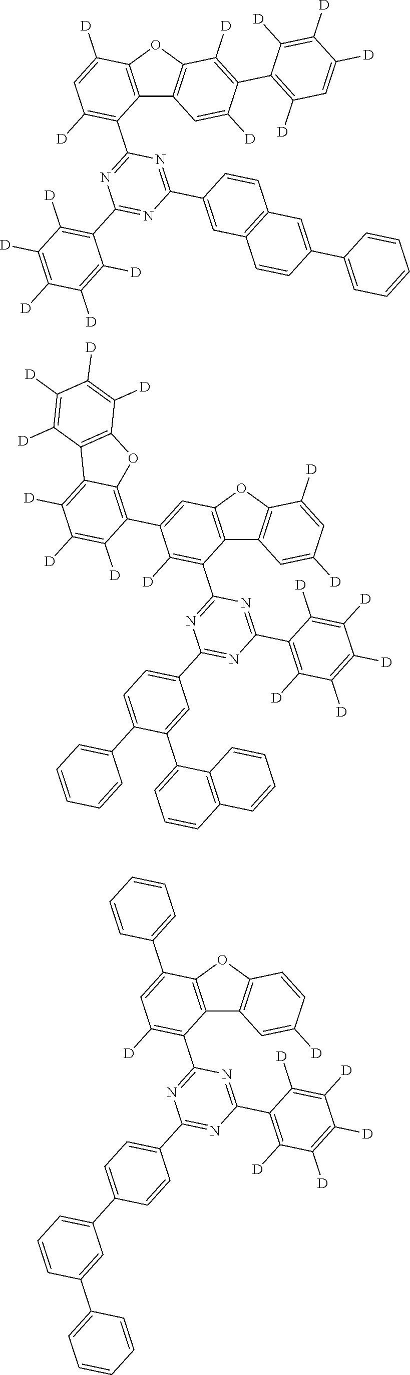

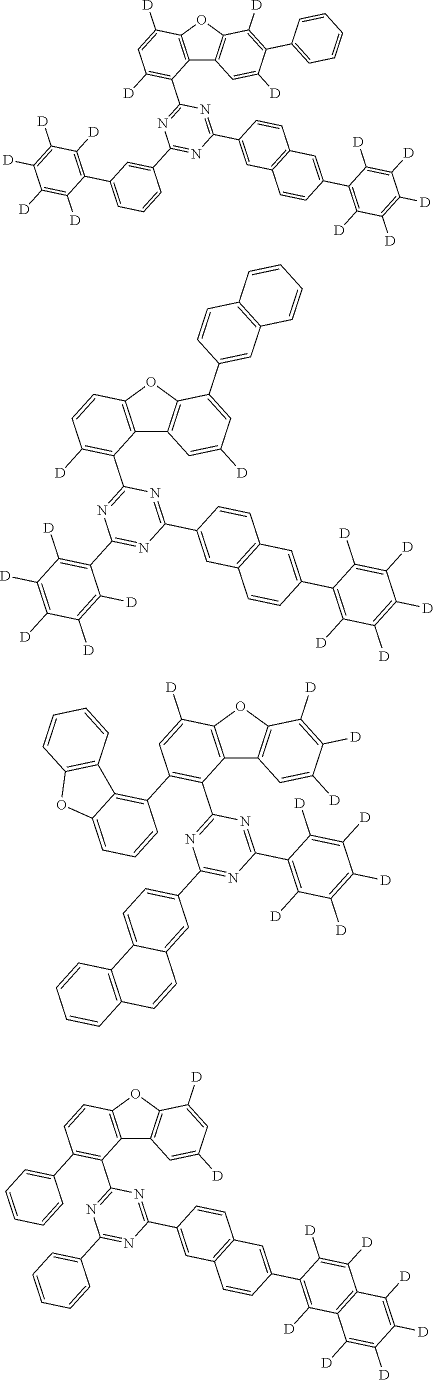

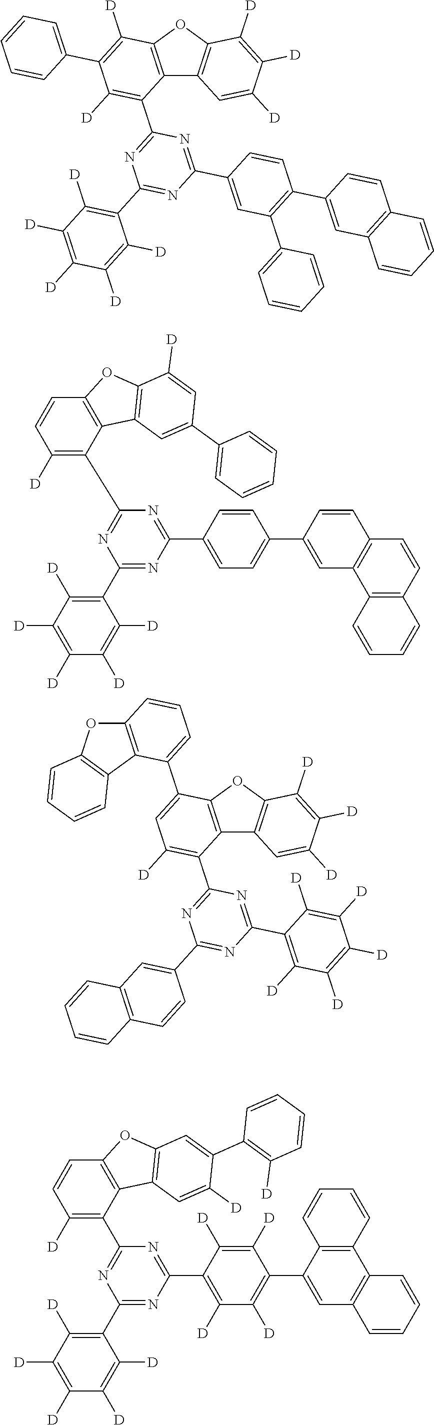

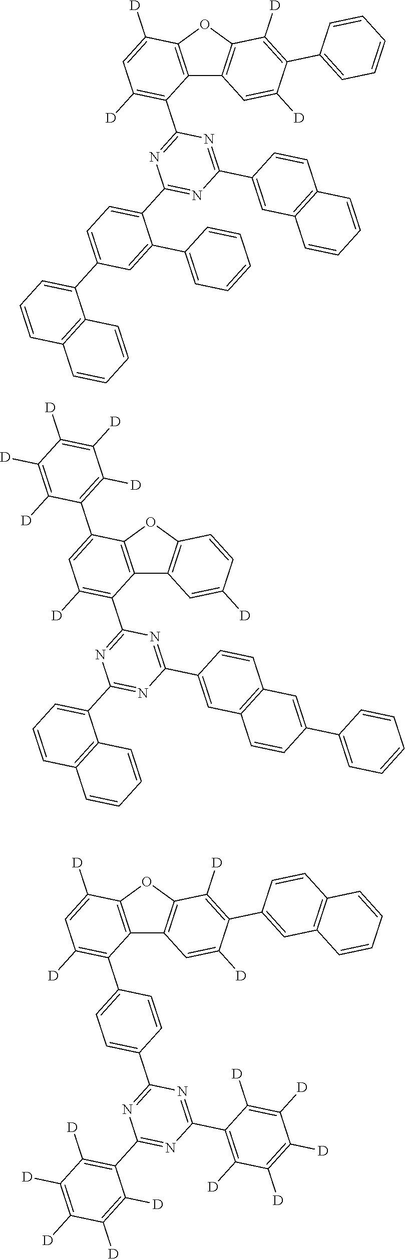

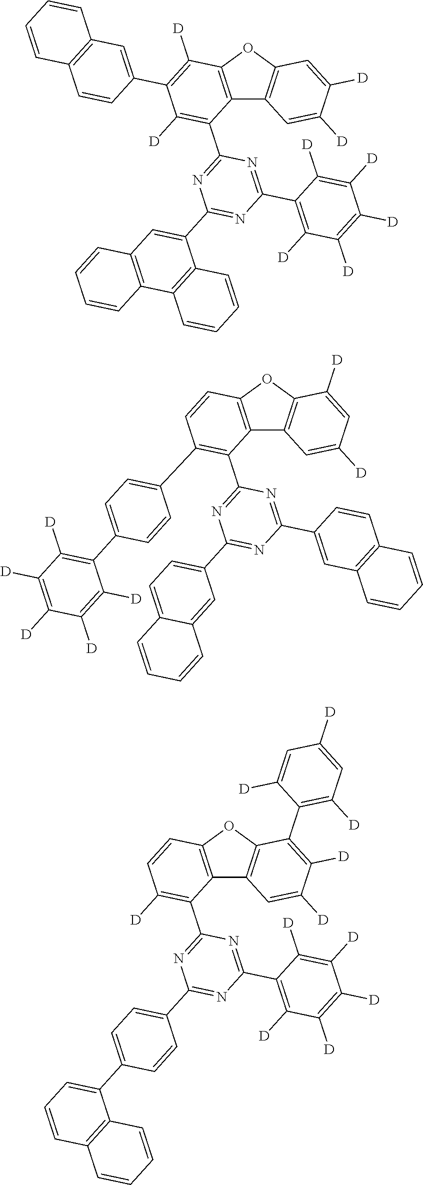 [SMILES: [2H]c1cc2oc3c([2H])c(-c4ccc5ccccc5c4)c([2H])c(-c4nc(-c5c([2H])c([2H])c([2H])c([2H])c5[2H])nc(-c5cc6ccccc6c6ccccc56)n4)c3c2cc1[2H] ; [2H]c1cc([2H])c2oc3ccc(-c4ccc(-c5c([2H])c([2H])c([2H])c([2H])c5[2H])cc4)c(-c4nc(-c5ccc6ccccc6c5)nc(-c5ccc6ccccc6c5)n4)c3c2c1 ; [2H]c1cc([2H])c(-c2c([2H])c([2H])cc3c2oc2ccc([2H])c(-c4nc(-c5ccc(-c6cccc7ccccc67)cc5)nc(-c5c([2H])c([2H])c([2H])c([2H])c5[2H])n4)c23)c([2H])c1]

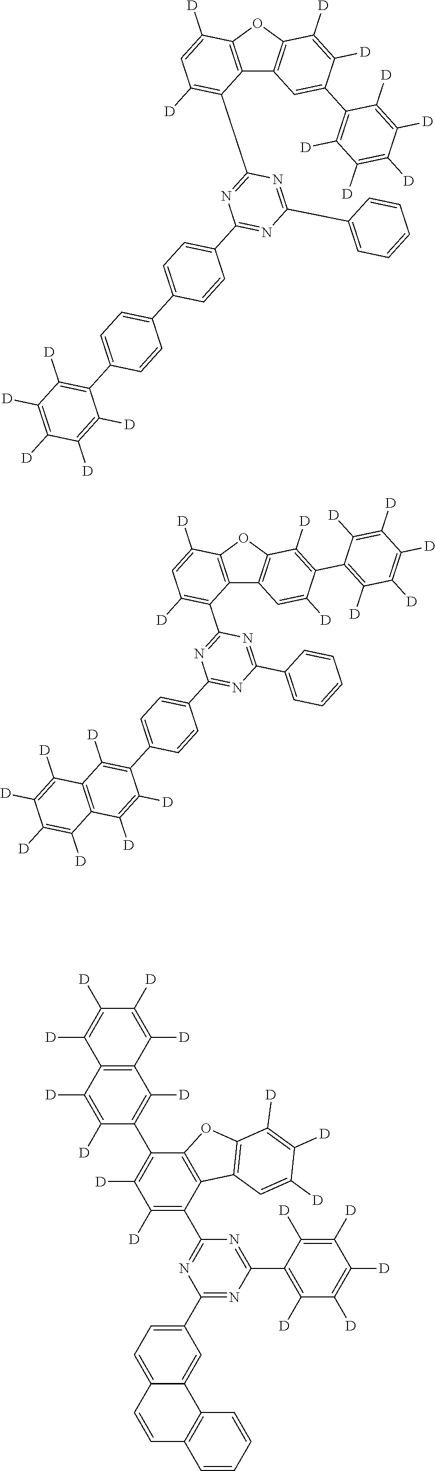

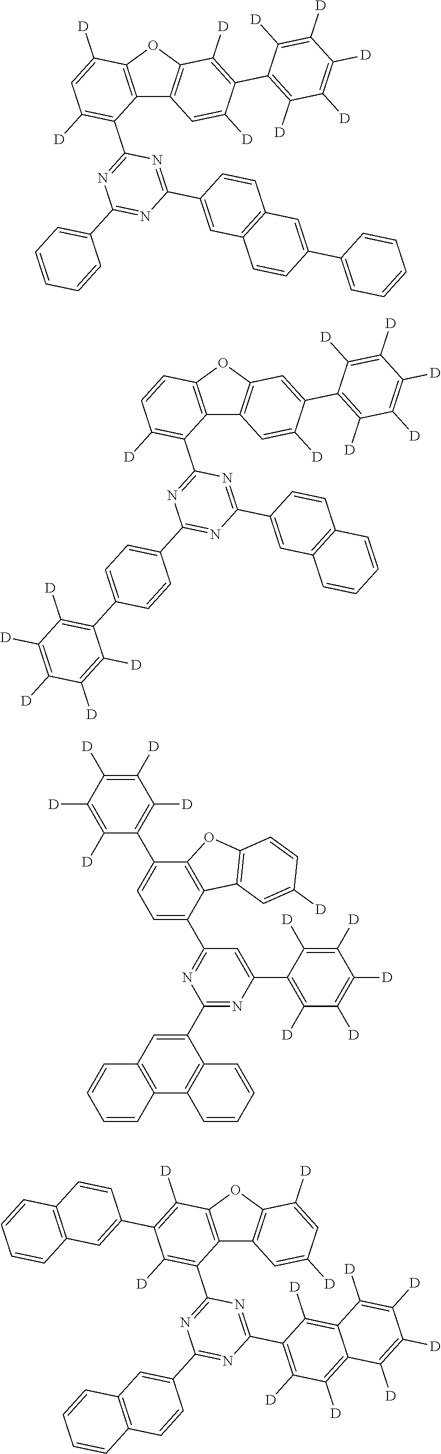

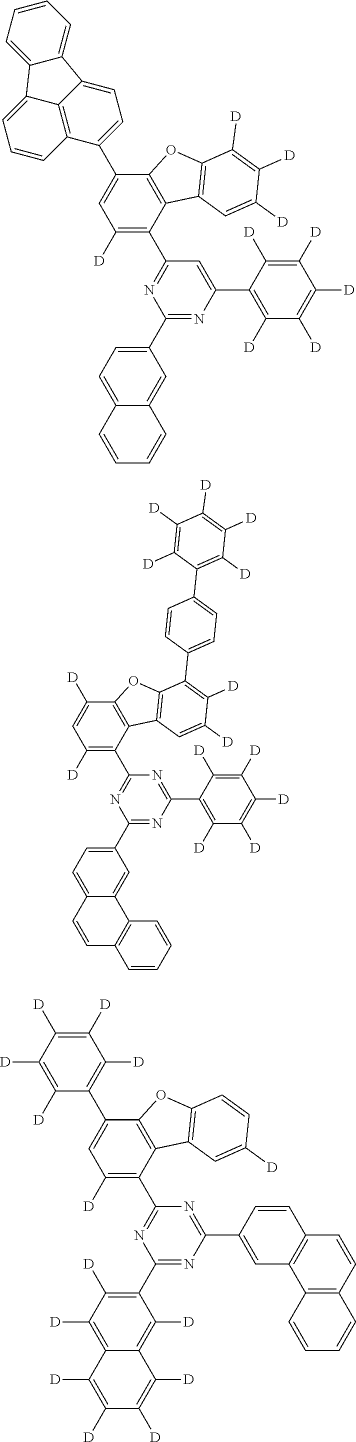

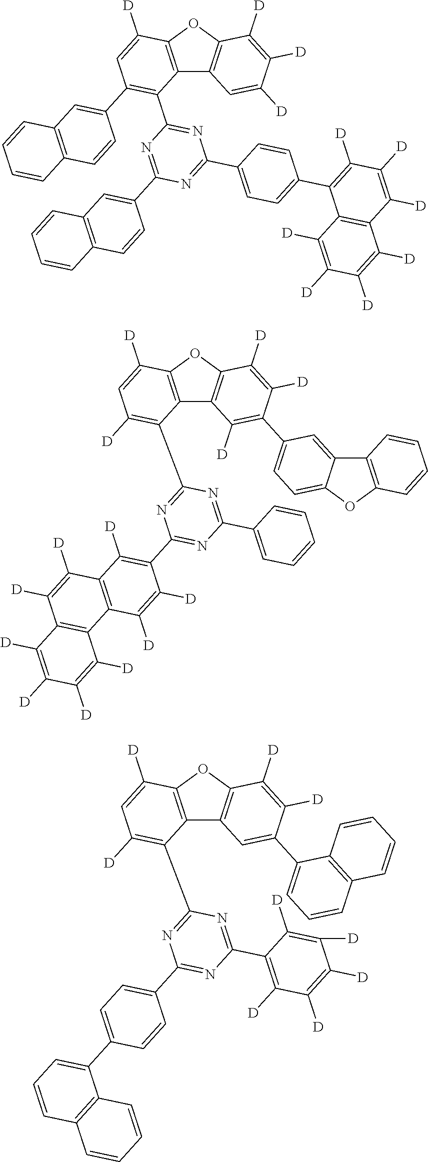 [SMILES: [2H]c1cc2c(oc3c([2H])cc(-c4ccc5ccccc5c4)c(-c4nc(-c5ccc(-c6c([2H])c([2H])c([2H])c7c([2H])c([2H])c([2H])c([2H])c67)cc5)nc(-c5ccc6ccccc6c5)n4)c32)c([2H])c1[2H] ; [2H]c1cc([2H])c2oc3c([2H])c([2H])c(-c4ccc5oc6ccccc6c5c4)c([2H])c3c2c1-c1nc(-c2ccccc2)nc(-c2c([2H])c([2H])c3c(c2[2H])c([2H])c([2H])c2c([2H])c([2H])c([2H])c([2H])c23)n1 ; [2H]c1cc([2H])c2oc3c([2H])c([2H])c(-c4cccc5ccccc45)cc3c2c1-c1nc(-c2ccc(-c3cccc4ccccc34)cc2)nc(-c2c([2H])c([2H])c([2H])c([2H])c2[2H])n1]

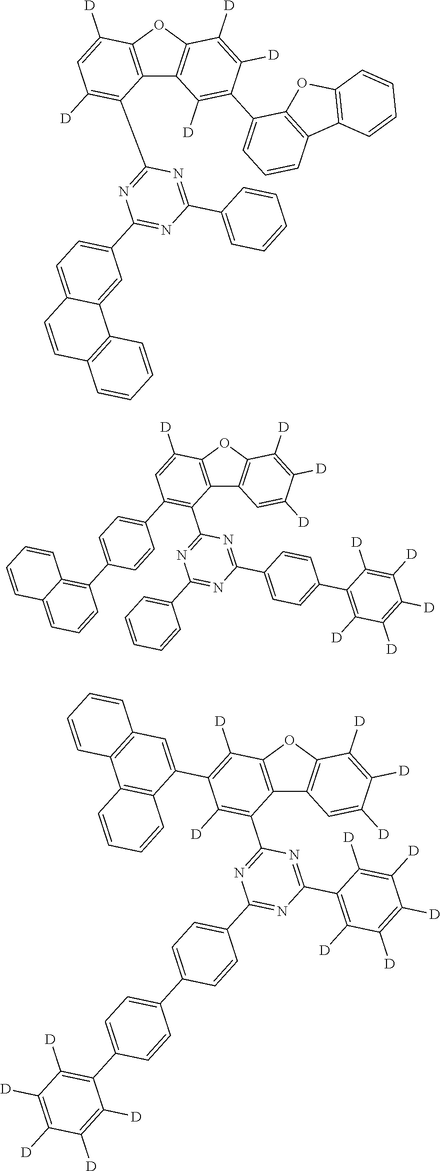

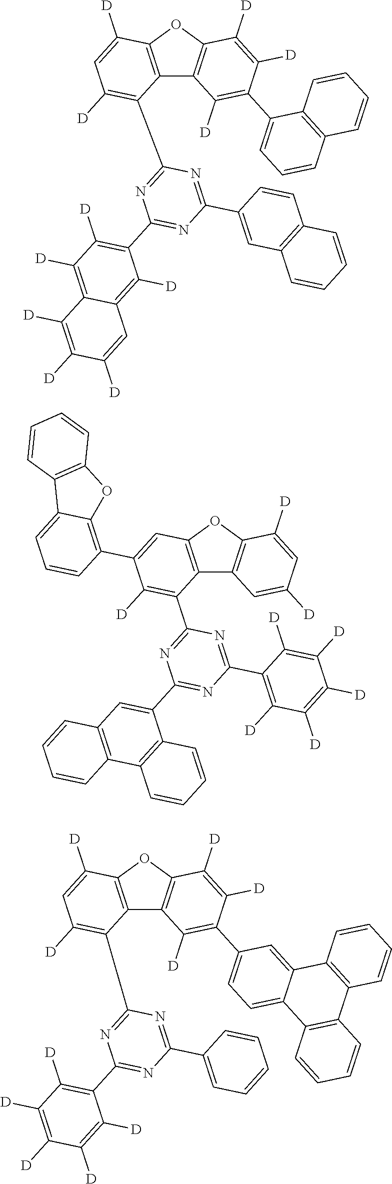

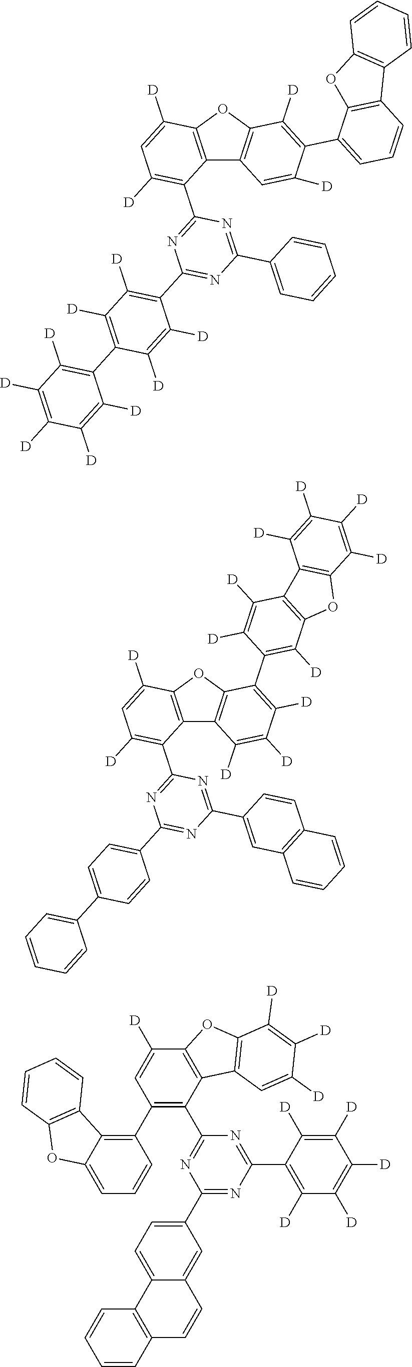 [SMILES: [2H]c1cc2c(oc3c([2H])cc([2H])c(-c4nc(-c5ccccc5)nc(-c5c([2H])c([2H])c(-c6c([2H])c([2H])c([2H])c([2H])c6[2H])c([2H])c5[2H])n4)c32)c([2H])c1-c1cccc2c1oc1ccccc12 ; [2H]c1cc([2H])c2oc3c(-c4c([2H])c([2H])c5c(oc6c([2H])c([2H])c([2H])c([2H])c65)c4[2H])c([2H])c([2H])c([2H])c3c2c1-c1nc(-c2ccc(-c3ccccc3)cc2)nc(-c2ccc3ccccc3c2)n1 ; [2H]c1cc2c(oc3c([2H])cc(-c4cccc5oc6ccccc6c45)c(-c4nc(-c5ccc6c(ccc7ccccc76)c5)nc(-c5c([2H])c([2H])c([2H])c([2H])c5[2H])n4)c32)c([2H])c1[2H]]

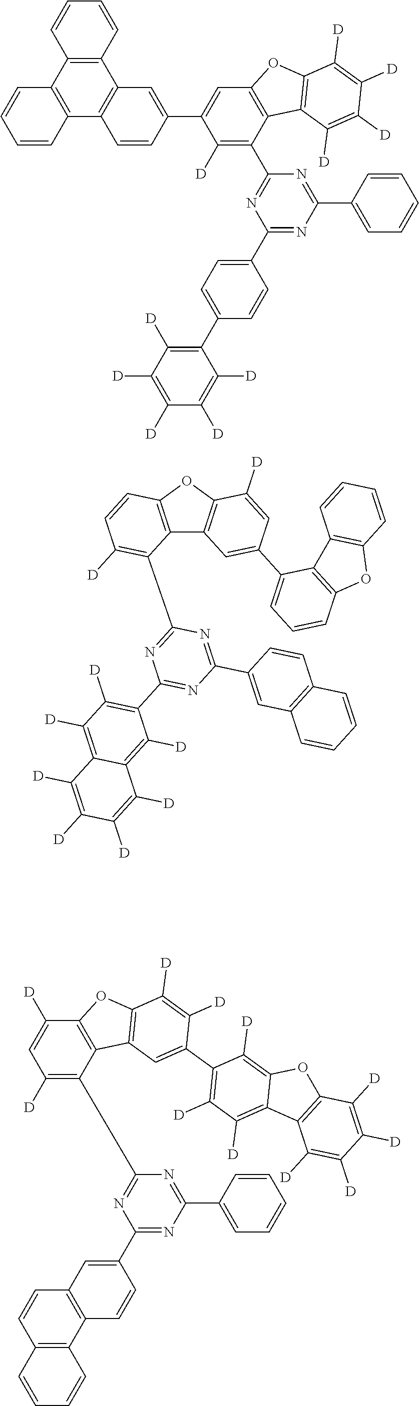 [SMILES: [2H]c1c([2H])c([2H])c(-c2ccc(-c3nc(-c4ccccc4)nc(-c4c([2H])c(-c5ccc6c7ccccc7c7ccccc7c6c5)cc5oc6c([2H])c([2H])c([2H])c([2H])c6c45)n3)cc2)c([2H])c1[2H] ; [2H]c1ccc2oc3c([2H])cc(-c4cccc5oc6ccccc6c45)cc3c2c1-c1nc(-c2ccc3ccccc3c2)nc(-c2c([2H])c([2H])c3c([2H])c([2H])c([2H])c([2H])c3c2[2H])n1 ; [2H]c1cc([2H])c2oc3c([2H])c([2H])c(-c4c([2H])c([2H])c5c(oc6c([2H])c([2H])c([2H])c([2H])c65)c4[2H])cc3c2c1-c1nc(-c2ccccc2)nc(-c2ccc3c(ccc4ccccc43)c2)n1]

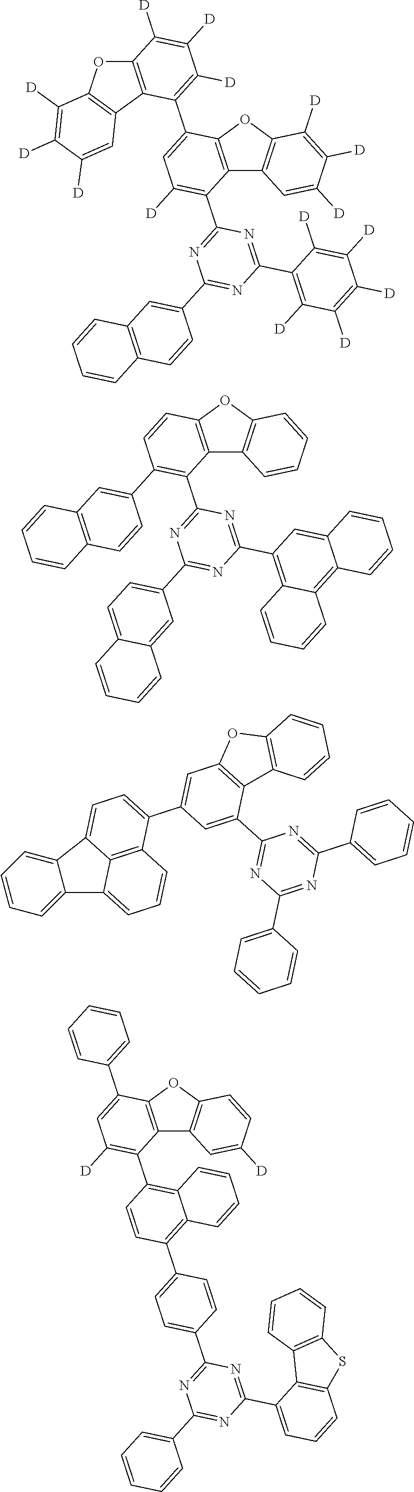 [SMILES: [2H]c1cc2c(oc3c(-c4c([2H])c([2H])c([2H])c5oc6c([2H])c([2H])c([2H])cc6c45)cc([2H])c(-c4nc(-c5ccc6ccccc6c5)nc(-c5c([2H])c([2H])c([2H])c([2H])c5[2H])n4)c32)c([2H])c1[2H] ; c1ccc2cc(-c3nc(-c4cc5ccccc5c5ccccc45)nc(-c4c(-c5ccc6ccccc6c5)ccc5oc6ccccc6c45)n3)ccc2c1 ; c1ccc(-c2nc(-c3ccccc3)nc(-c3cc(-c4ccc5c6c(cccc46)-c4ccccc4-5)cc4oc5ccccc5c34)n2)cc1 ; [2H]c1ccc2oc3c(-c4ccccc4)cc([2H])c(-c4ccc(-c5ccc(-c6nc(-c7ccccc7)nc(-c7cccc8sc9ccccc9c78)n6)cc5)c5ccccc45)c3c2c1]

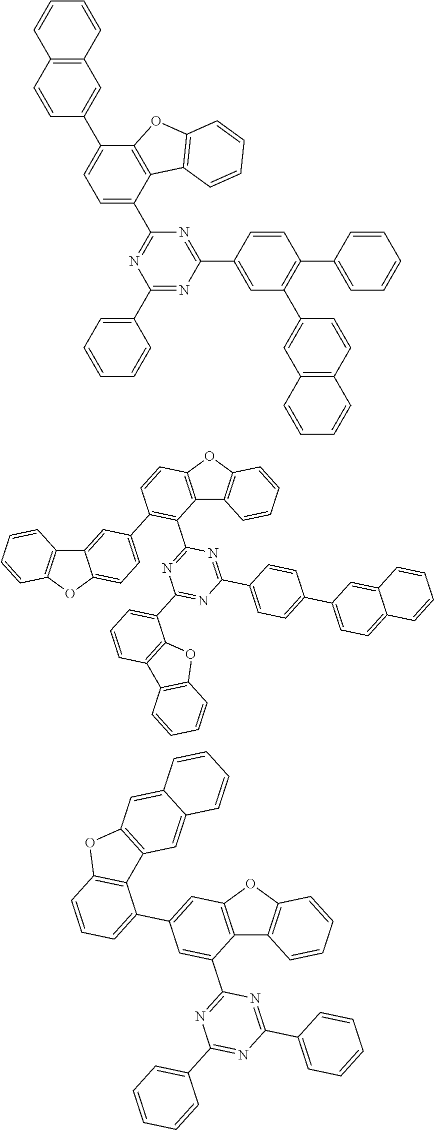

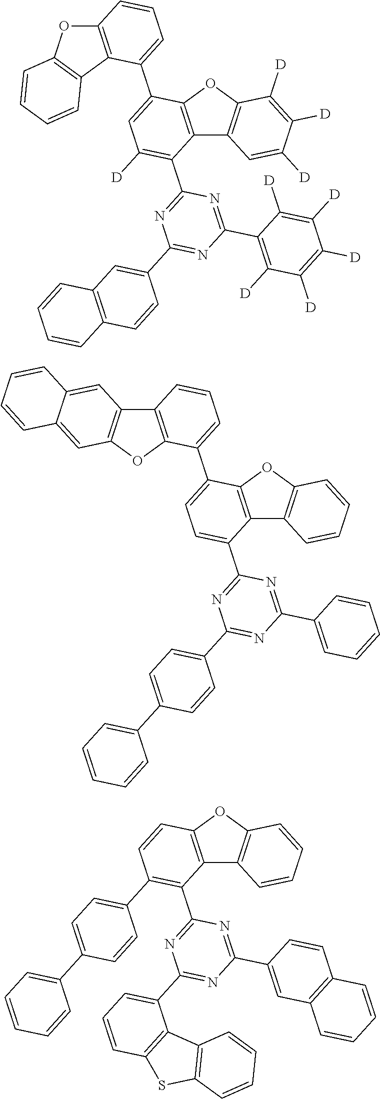

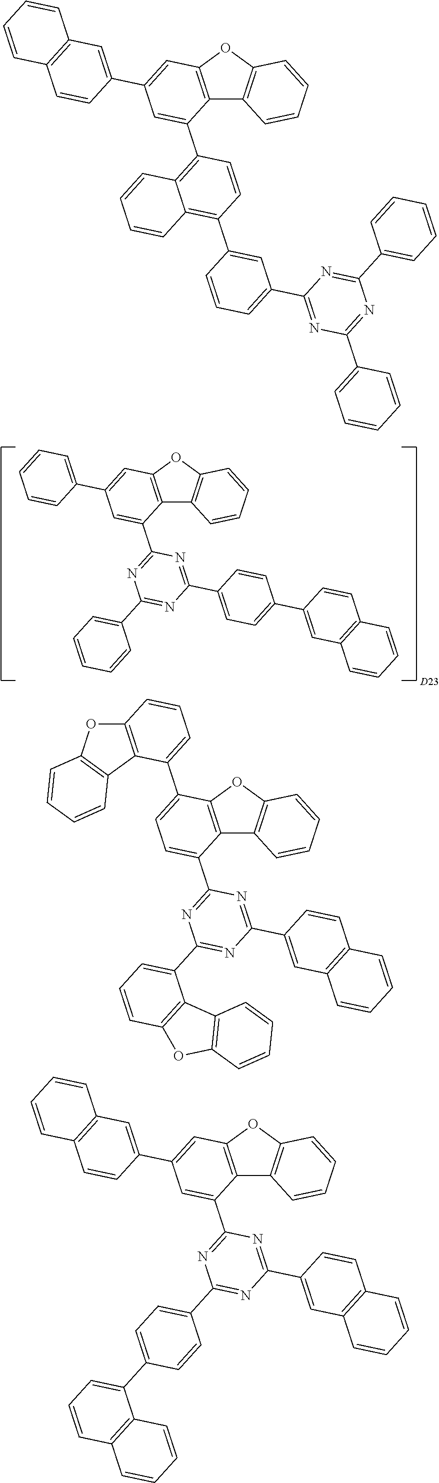

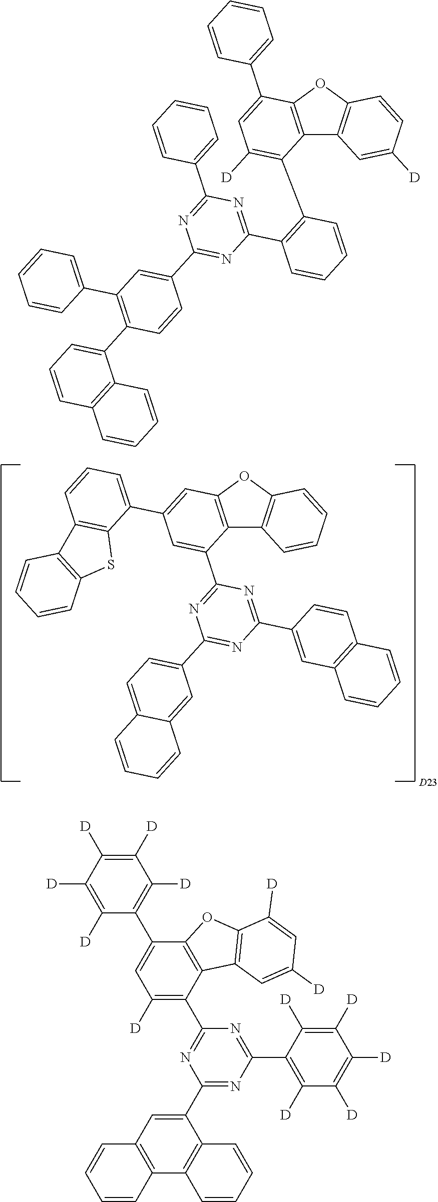

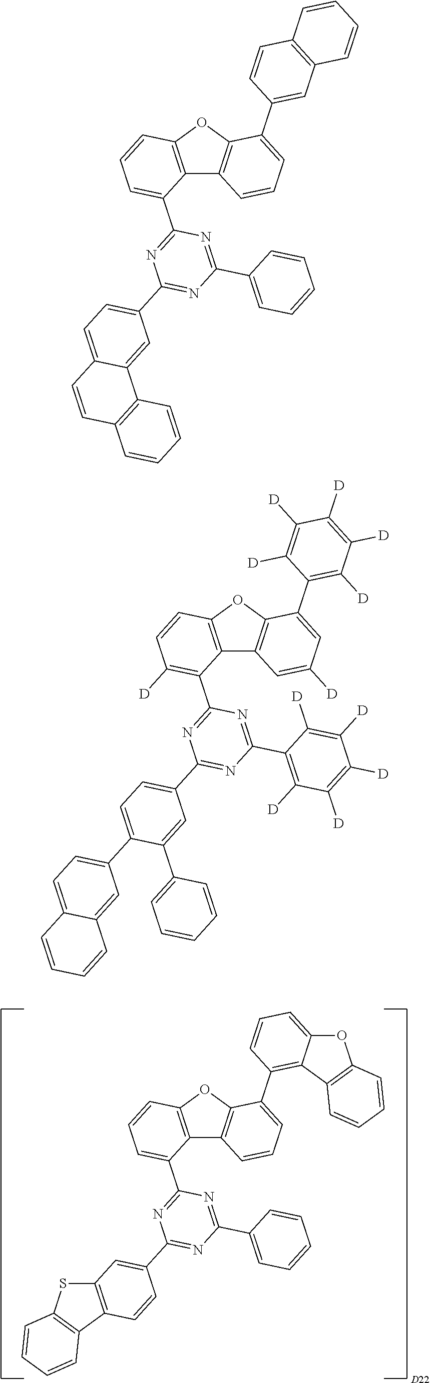

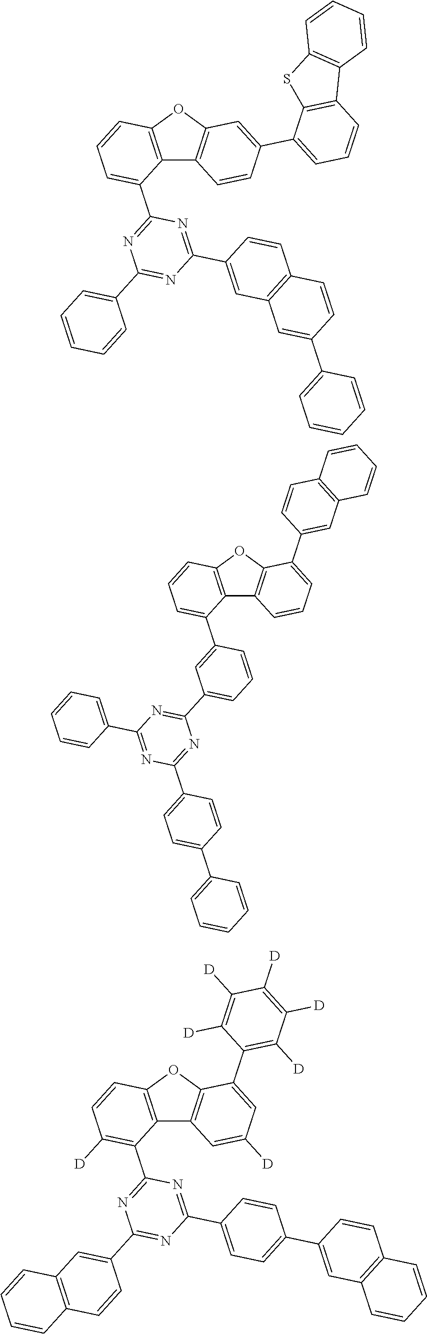

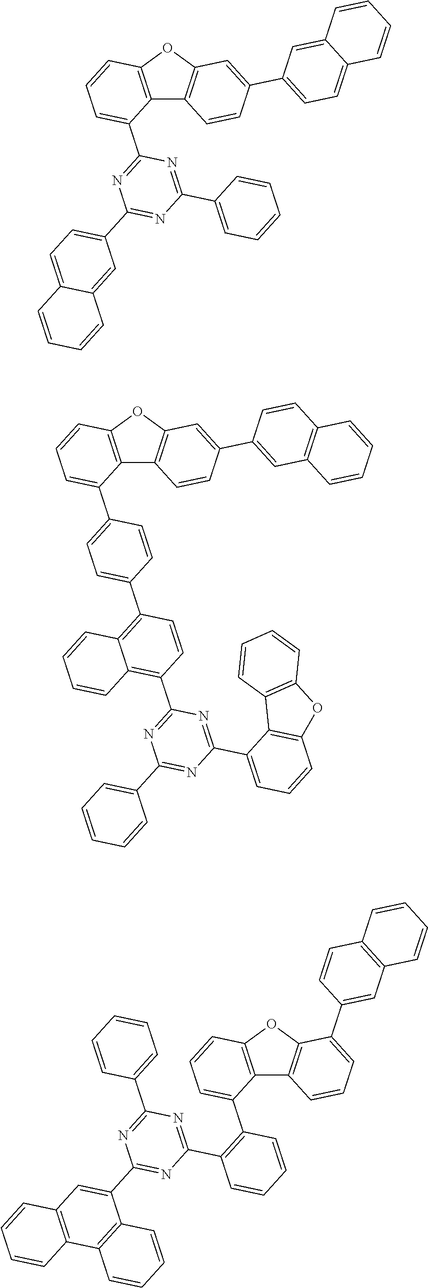 [SMILES: c1ccc(-c2nc(-c3ccc4ccccc4c3)nc(-c3cccc4oc5cc(-c6ccc7ccccc7c6)ccc5c34)n2)cc1 ; c1ccc(-c2nc(-c3ccc(-c4ccc(-c5cccc6oc7cc(-c8ccc9ccccc9c8)ccc7c56)cc4)c4ccccc34)nc(-c3cccc4oc5ccccc5c34)n2)cc1 ; c1ccc(-c2nc(-c3ccccc3-c3cccc4oc5c(-c6ccc7ccccc7c6)cccc5c34)nc(-c3cc4ccccc4c4ccccc34)n2)cc1]

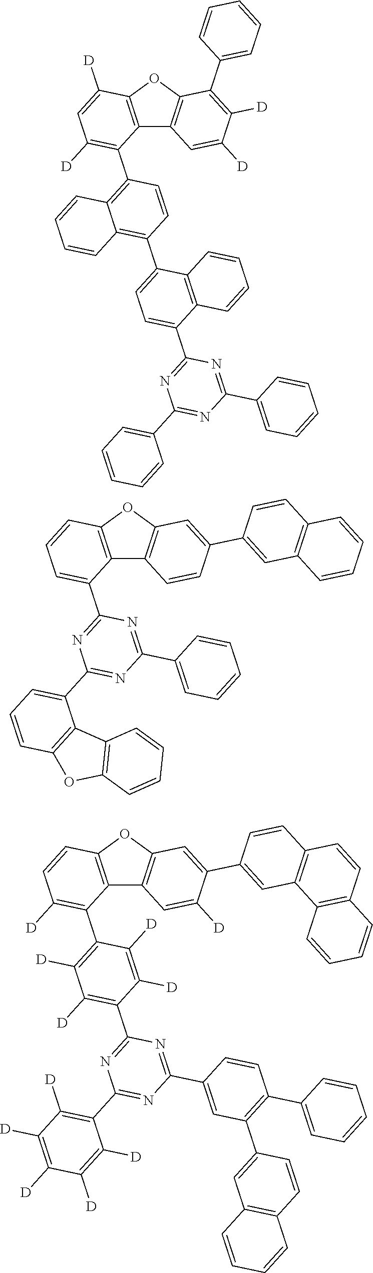

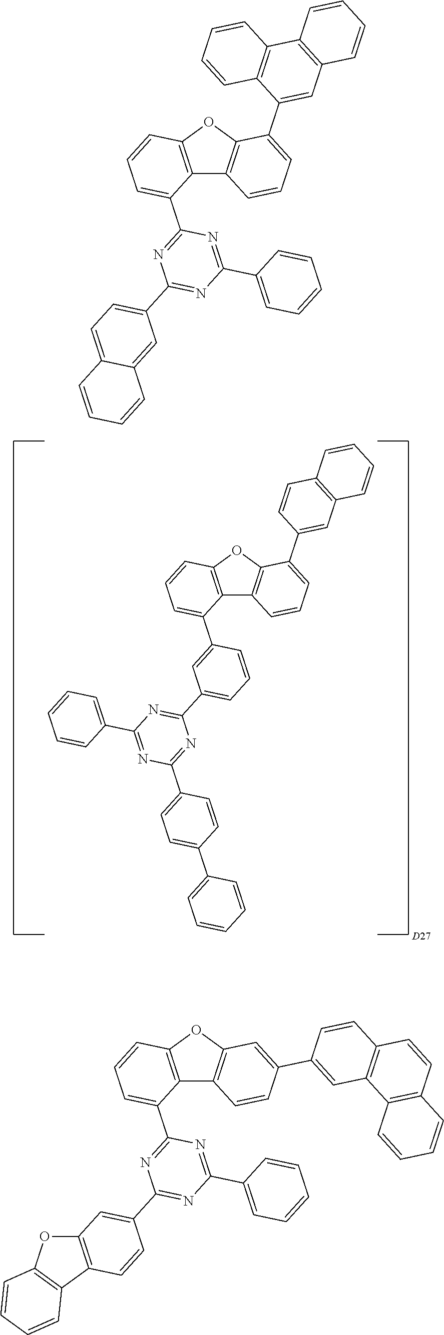

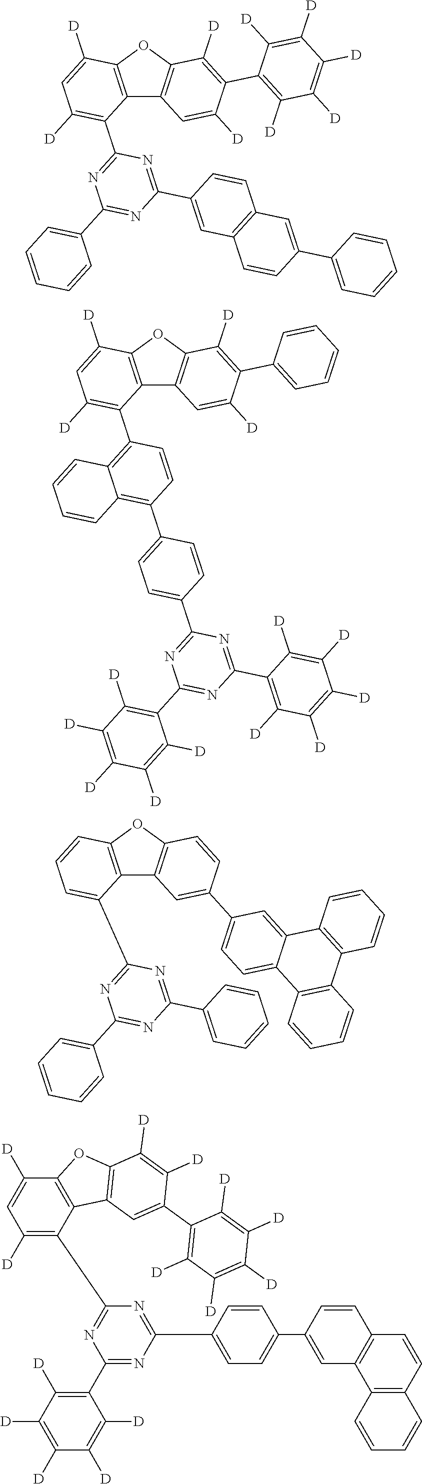

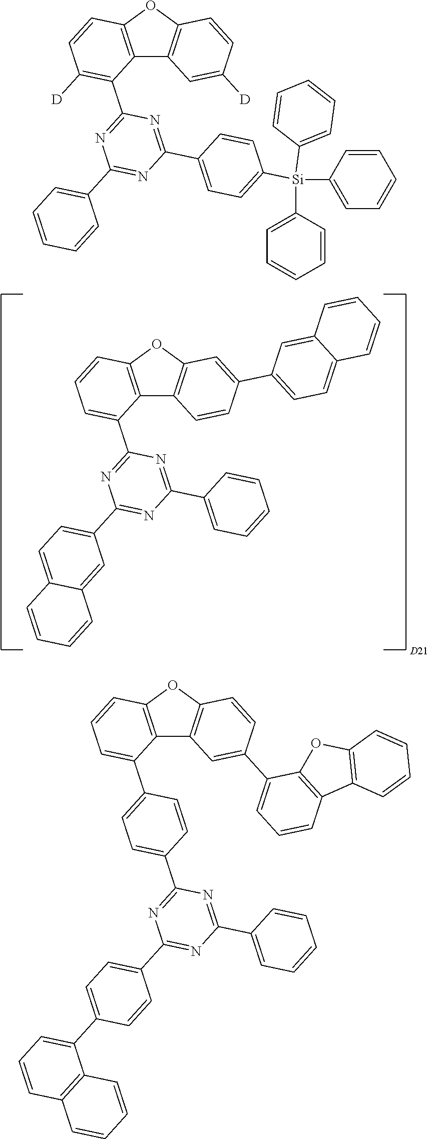

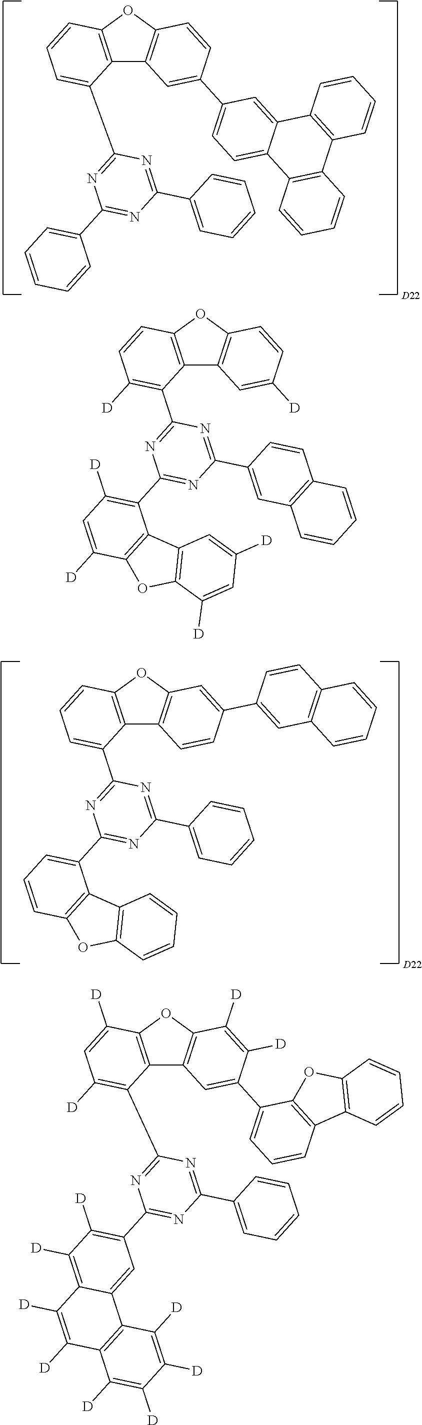

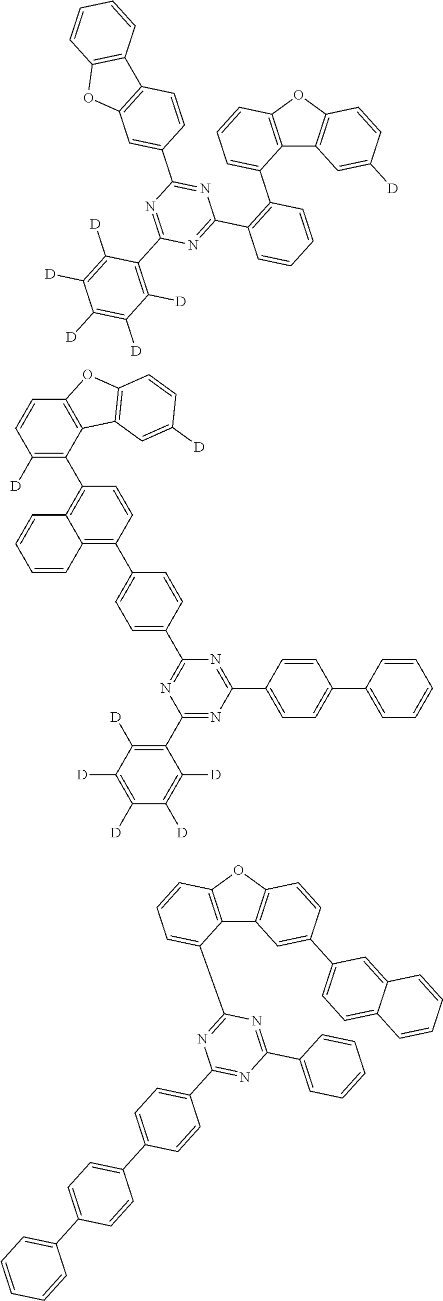

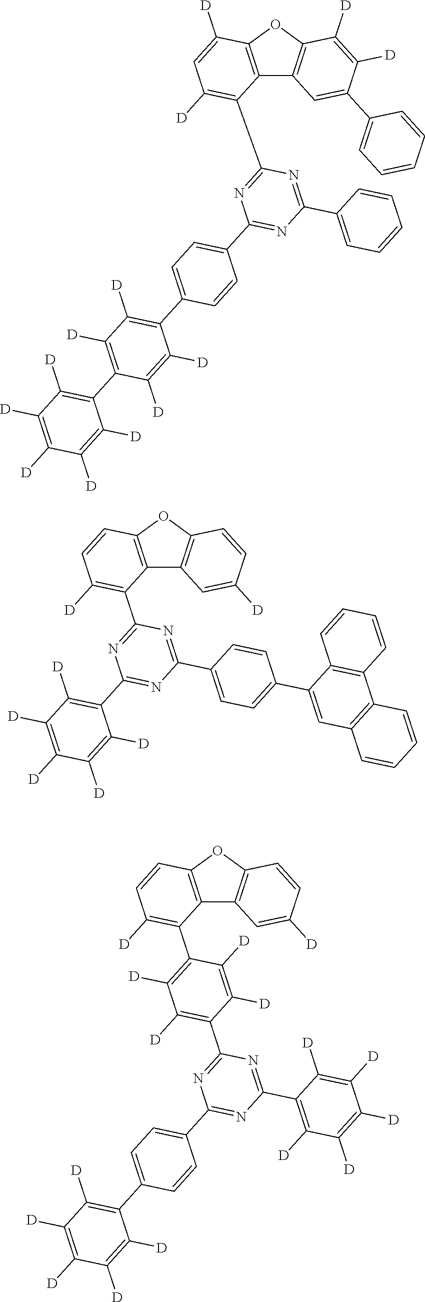

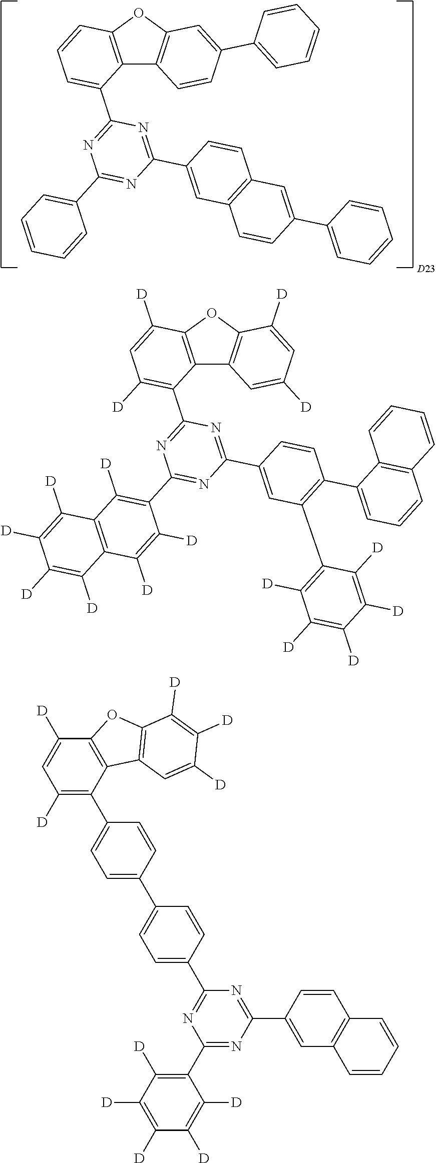

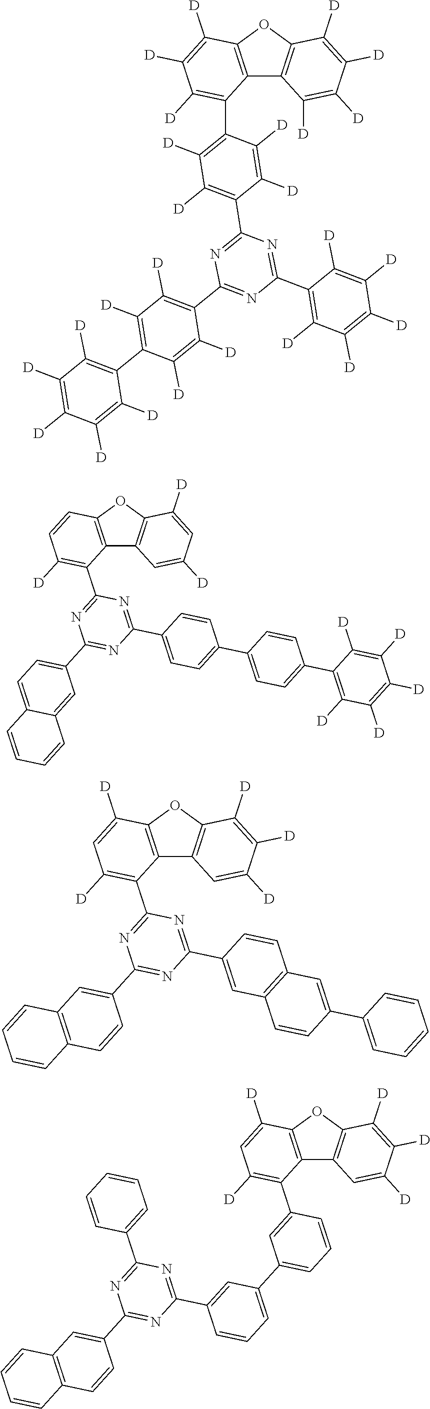

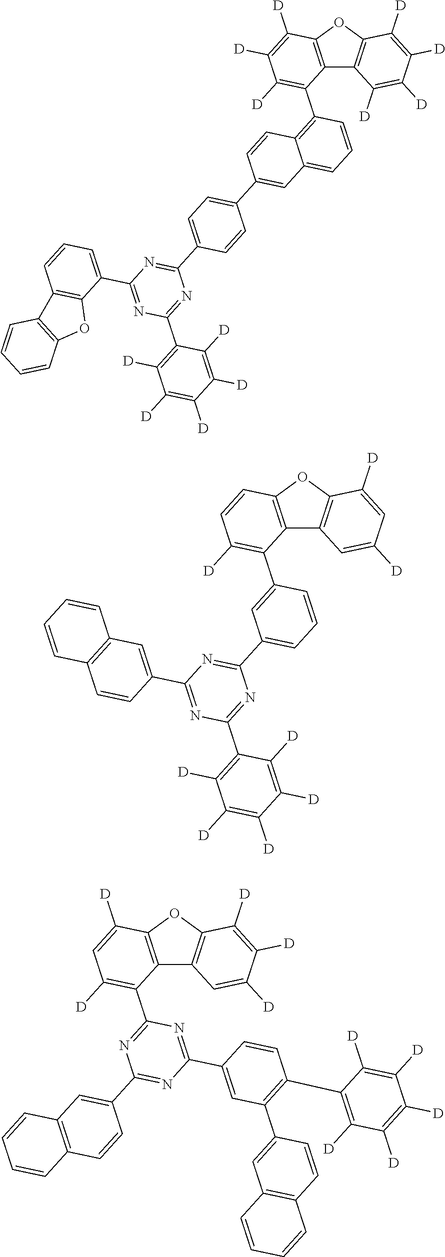 [SMILES: [2H]c1c([2H])c([2H])c(-c2nc(-c3ccc(-c4ccc5c(-c6c([2H])c([2H])c([2H])c7oc8c([2H])c([2H])c([2H])c([2H])c8c67)cccc5c4)cc3)nc(-c3cccc4c3oc3ccccc34)n2)c([2H])c1[2H] ; [2H]c1cc([2H])c2oc3ccc([2H])c(-c4cccc(-c5nc(-c6ccc7ccccc7c6)nc(-c6c([2H])c([2H])c([2H])c([2H])c6[2H])n5)c4)c3c2c1 ; [2H]c1cc2c(oc3c([2H])cc([2H])c(-c4nc(-c5ccc(-c6c([2H])c([2H])c([2H])c([2H])c6[2H])c(-c6ccc7ccccc7c6)c5)nc(-c5ccc6ccccc6c5)n4)c32)c([2H])c1[2H]]

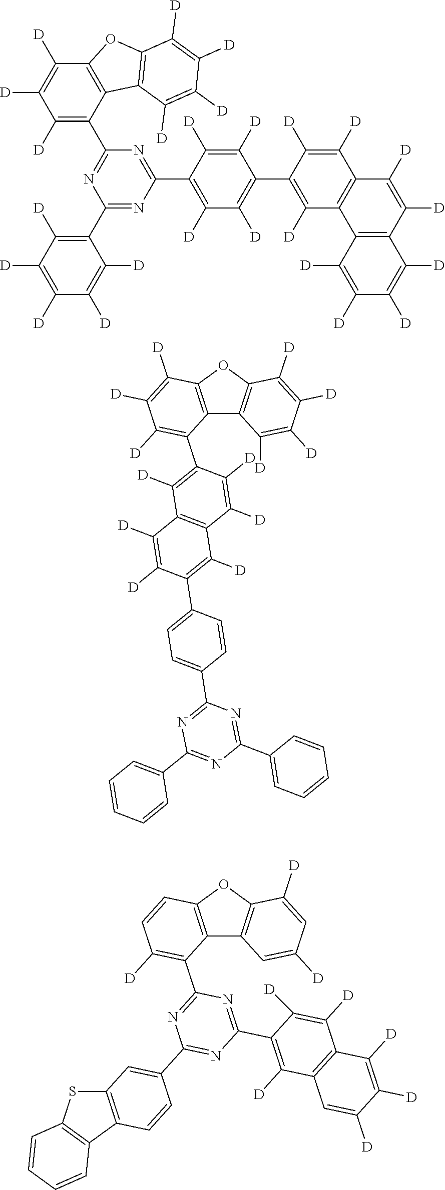 [SMILES: [2H]c1c([2H])c([2H])c(-c2nc(-c3c([2H])c([2H])c(-c4c([2H])c([2H])c5c([2H])c([2H])c6c([2H])c([2H])c([2H])c([2H])c6c5c4[2H])c([2H])c3[2H])nc(-c3c([2H])c([2H])c([2H])c4oc5c([2H])c([2H])c([2H])c([2H])c5c34)n2)c([2H])c1[2H] ; [2H]c1c([2H])c([2H])c2c(oc3c([2H])c([2H])c([2H])c(-c4c([2H])c([2H])c5c([2H])c(-c6ccc(-c7nc(-c8ccccc8)nc(-c8ccccc8)n7)cc6)c([2H])c([2H])c5c4[2H])c32)c1[2H] ; [2H]c1cc([2H])c2oc3ccc([2H])c(-c4nc(-c5ccc6c(c5)sc5ccccc56)nc(-c5c([2H])c([2H])c6c([2H])c([2H])c([2H])cc6c5[2H])n4)c3c2c1]

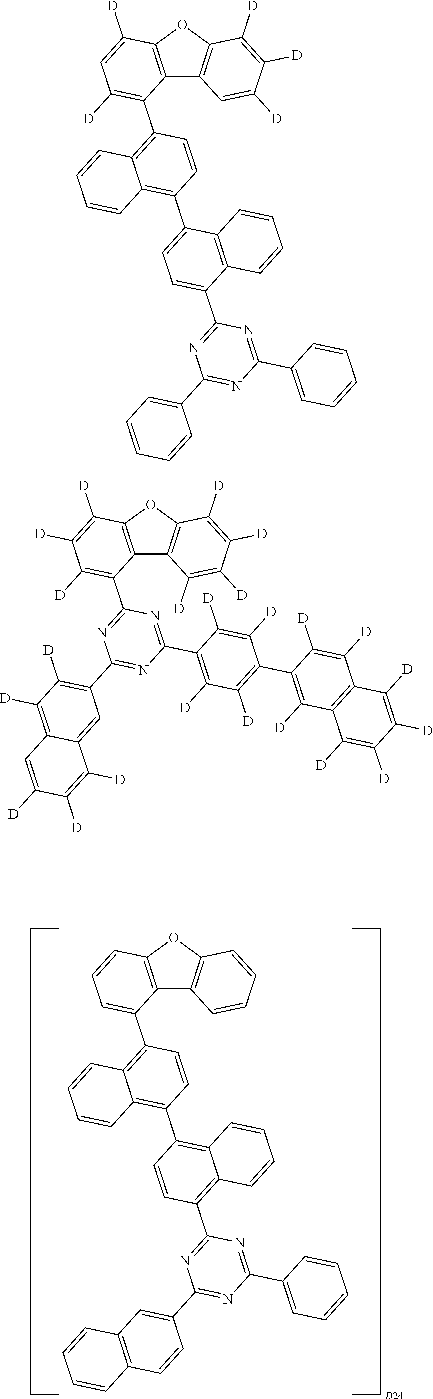

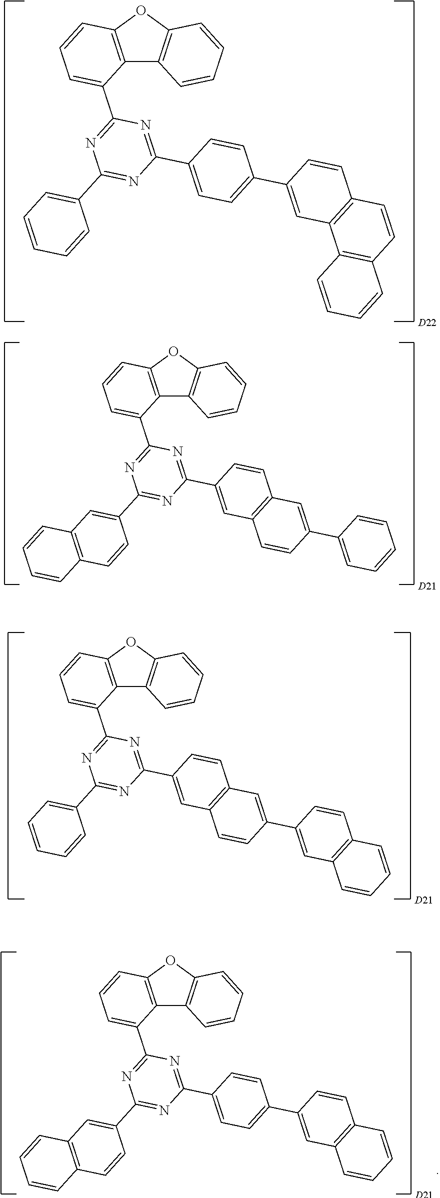

6. The organic light emitting device according to claim 1, wherein
Ar₃ and Ar₄ are each independently phenyl, biphenylyl, terphenylyl, quaterphenylyl, triphenylmethyl phenyl, triphenylsilyl phenyl, naphthyl, phenanthrenyl, triphenylenyl, fluoranthenyl, chrysenyl, benzo[c]phenanthrenyl, carbazolyl, phenyl carbazolyl, dimethylfluorenyl, dibenzofuranyl, or dibenzothiophenyl.

7. The organic light emitting device according to claim 1, wherein
L₄ is phenylene, biphenyldiyl, terphenyldiyl, quarterphenyldiyl, naphthalenediyl, phenylnaphthalenediyl, or phenylnaphthalenediyl substituted with one phenyl.

8. The organic light emitting device according to claim 1, wherein
L₅ and L₆ are each independently a single bond, phenylene, biphenyldiyl, naphthalenediyl, phenylnaphthalenediyl, or carbazolediyl.

9. The organic light emitting device according to claim 1, wherein
the compound represented by Chemical Formula 2 is one selected from the following: 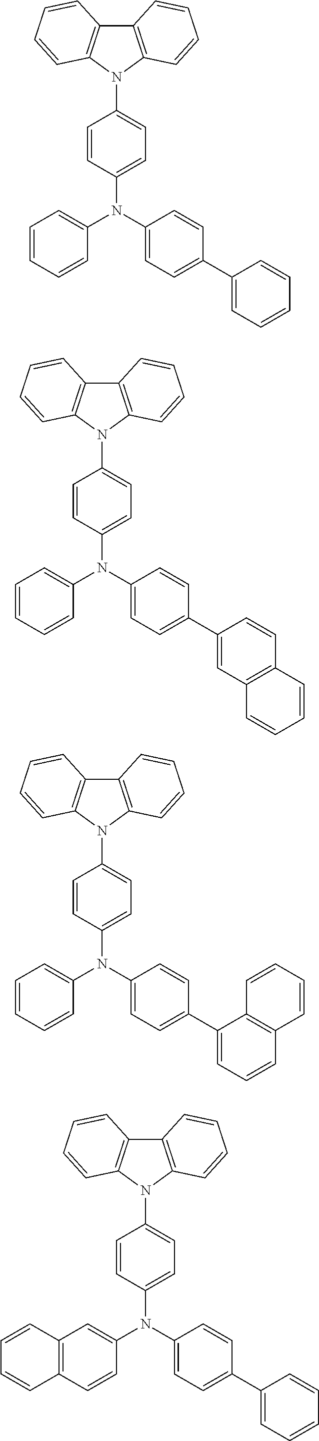 [SMILES: c1ccc(-c2ccc(N(c3ccccc3)c3ccc(-n4c5ccccc5c5ccccc54)cc3)cc2)cc1 ; c1ccc(N(c2ccc(-c3ccc4ccccc4c3)cc2)c2ccc(-n3c4ccccc4c4ccccc43)cc2)cc1 ; c1ccc(N(c2ccc(-c3cccc4ccccc34)cc2)c2ccc(-n3c4ccccc4c4ccccc43)cc2)cc1 ; c1ccc(-c2ccc(N(c3ccc(-n4c5ccccc5c5ccccc54)cc3)c3ccc4ccccc4c3)cc2)cc1] 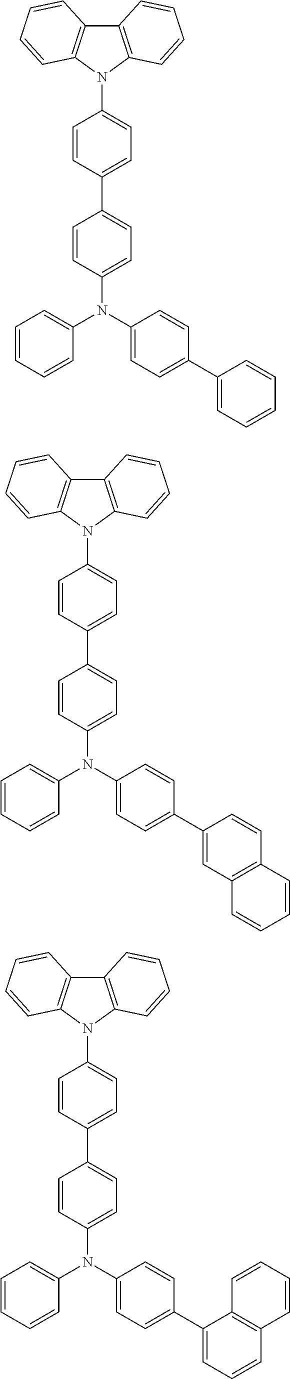 [SMILES: c1ccc(-c2ccc(N(c3ccccc3)c3ccc(-c4ccc(-n5c6ccccc6c6ccccc65)cc4)cc3)cc2)cc1 ; c1ccc(N(c2ccc(-c3ccc(-n4c5ccccc5c5ccccc54)cc3)cc2)c2ccc(-c3ccc4ccccc4c3)cc2)cc1 ; c1ccc(N(c2ccc(-c3ccc(-n4c5ccccc5c5ccccc54)cc3)cc2)c2ccc(-c3cccc4ccccc34)cc2)cc1] 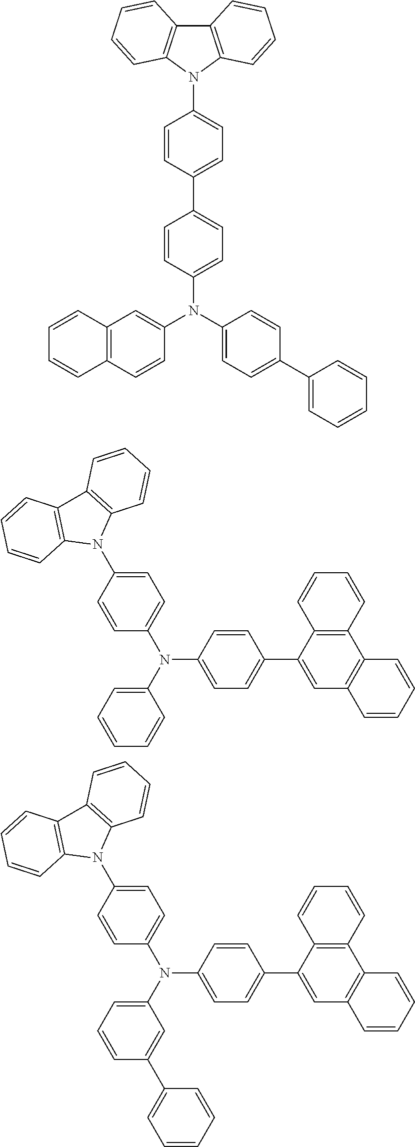 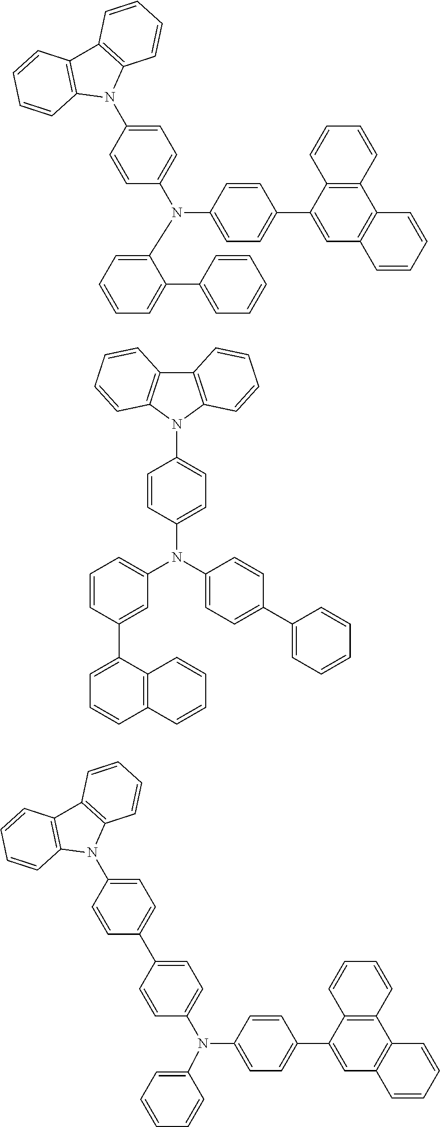 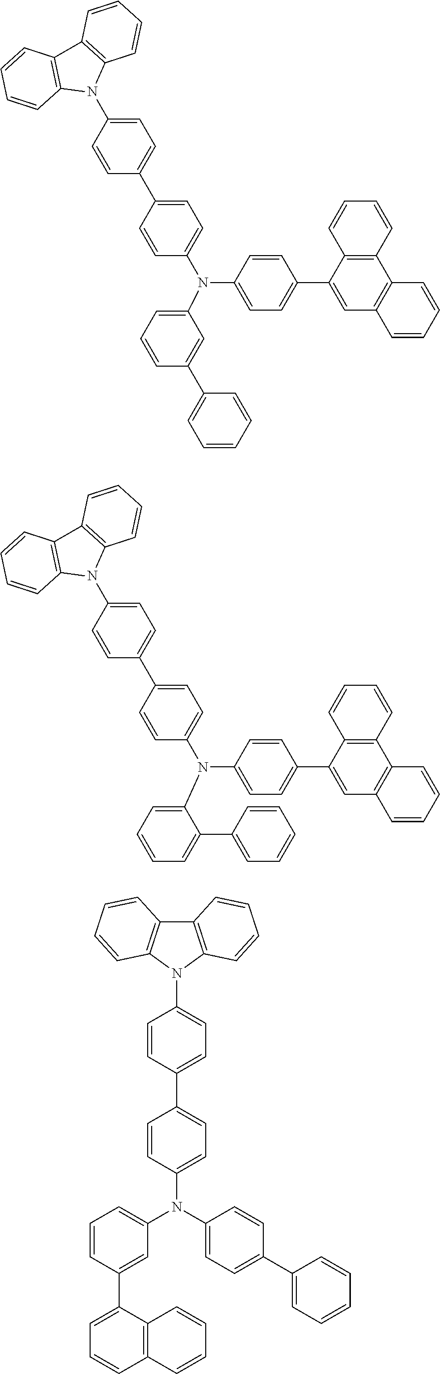 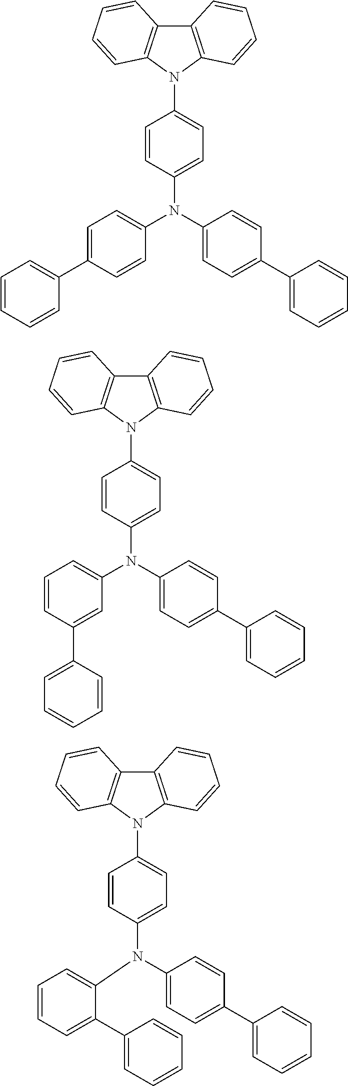 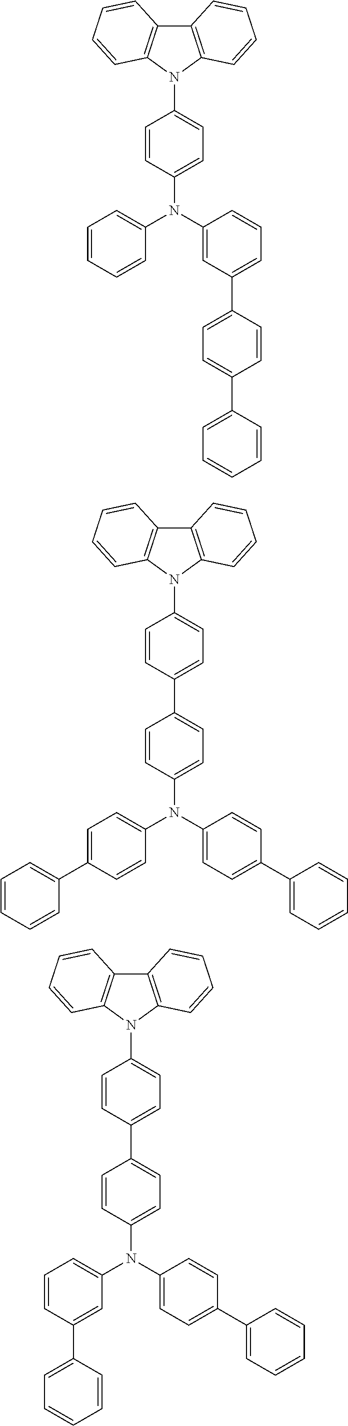 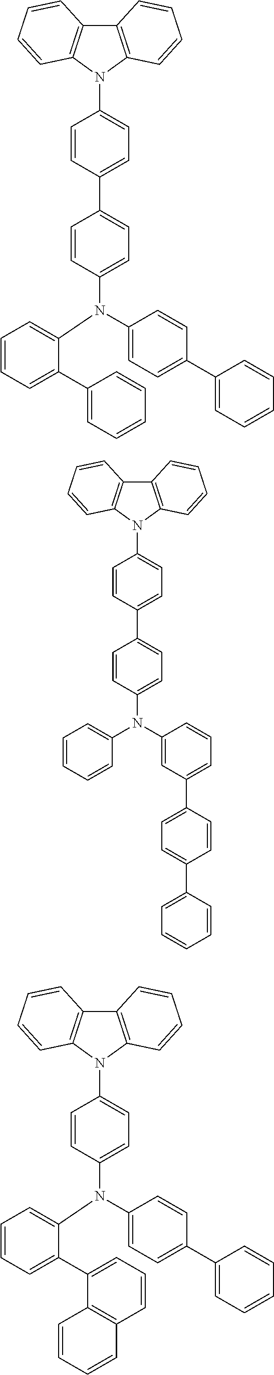 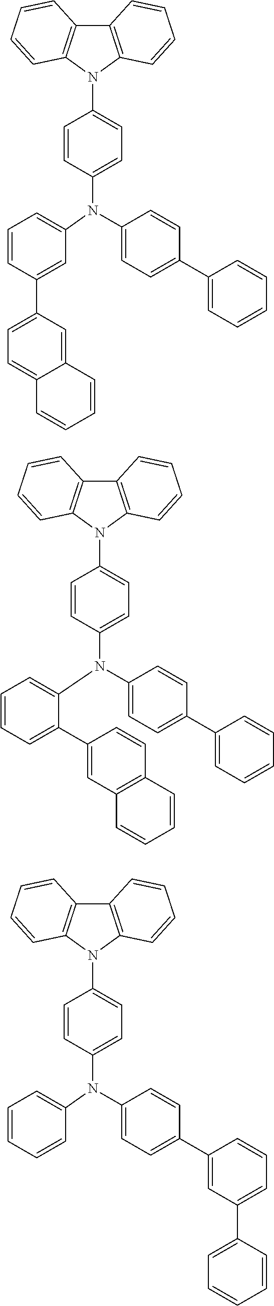 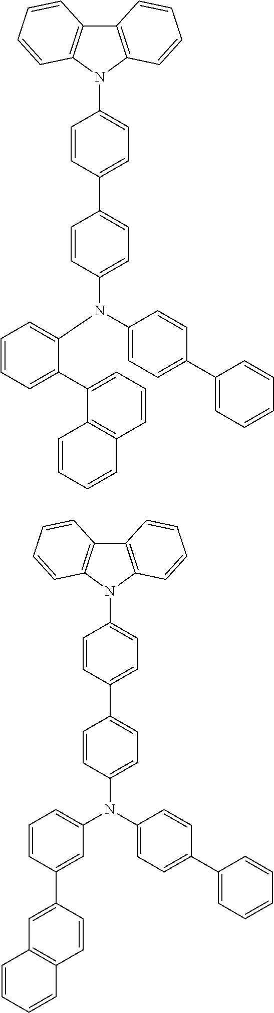 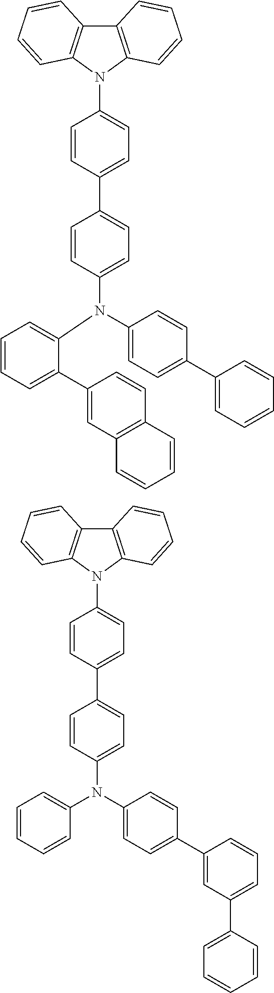 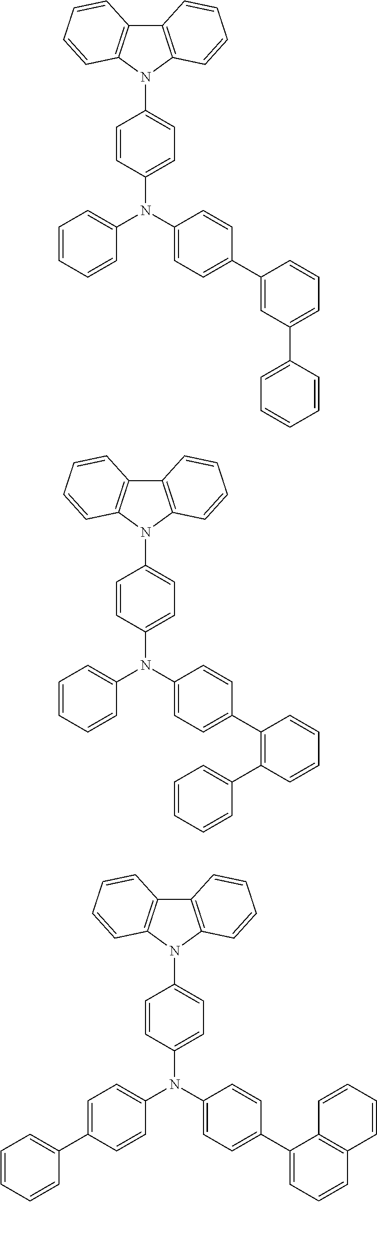 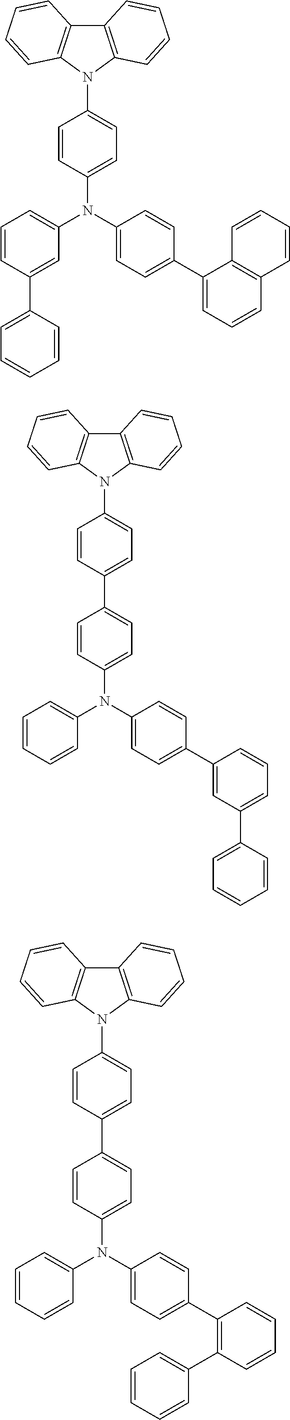 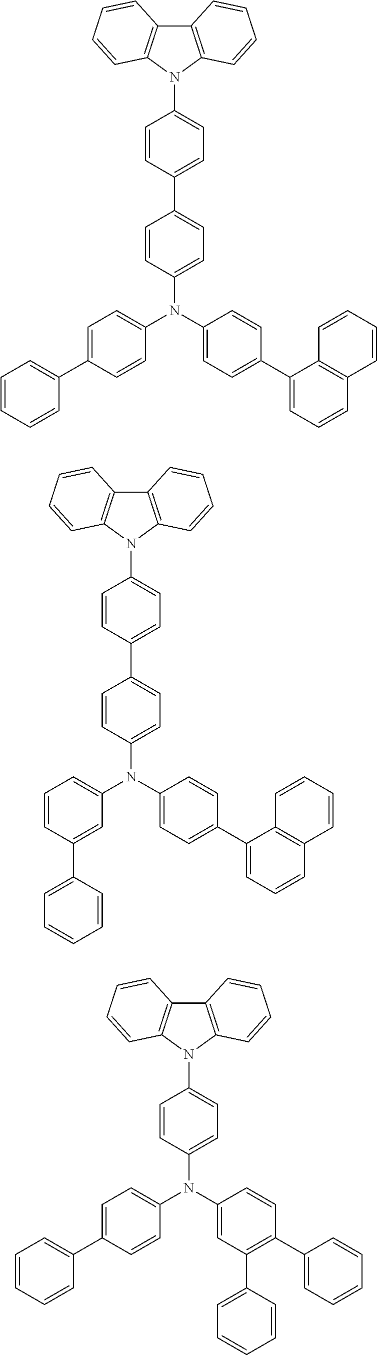 [SMILES: c1ccc(-c2ccc(N(c3ccc(-c4ccc(-n5c6ccccc6c6ccccc65)cc4)cc3)c3ccc(-c4cccc5ccccc45)cc3)cc2)cc1 ; c1ccc(-c2cccc(N(c3ccc(-c4ccc(-n5c6ccccc6c6ccccc65)cc4)cc3)c3ccc(-c4cccc5ccccc45)cc3)c2)cc1 ; c1ccc(-c2ccc(N(c3ccc(-n4c5ccccc5c5ccccc54)cc3)c3ccc(-c4ccccc4)c(-c4ccccc4)c3)cc2)cc1] 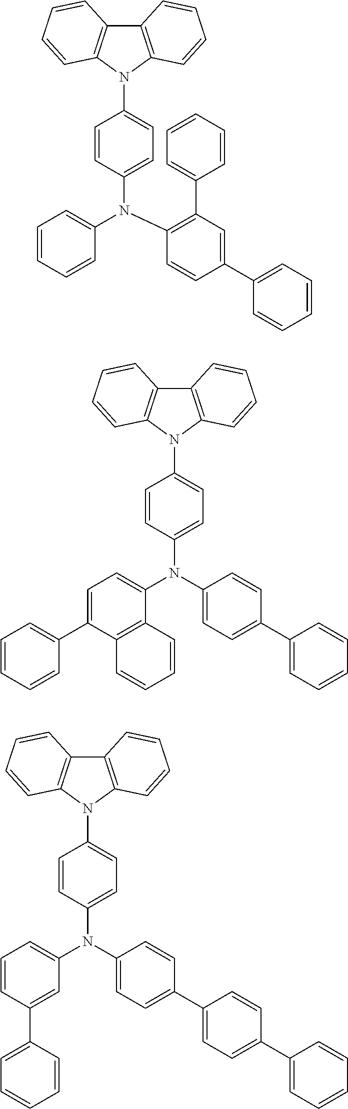 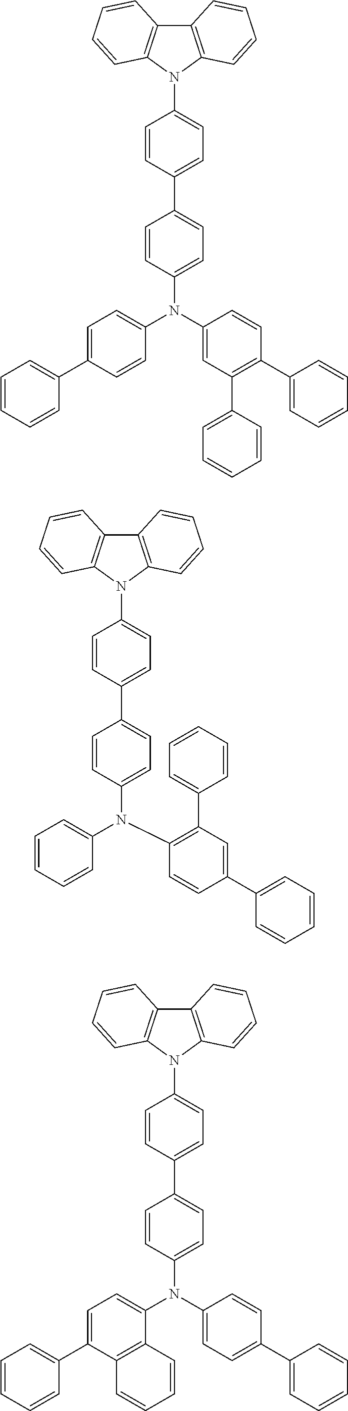 [SMILES: c1ccc(-c2ccc(N(c3ccc(-c4ccc(-n5c6ccccc6c6ccccc65)cc4)cc3)c3ccc(-c4ccccc4)c(-c4ccccc4)c3)cc2)cc1 ; c1ccc(-c2ccc(N(c3ccccc3)c3ccc(-c4ccc(-n5c6ccccc6c6ccccc65)cc4)cc3)c(-c3ccccc3)c2)cc1 ; c1ccc(-c2ccc(N(c3ccc(-c4ccc(-n5c6ccccc6c6ccccc65)cc4)cc3)c3ccc(-c4ccccc4)c4ccccc34)cc2)cc1] 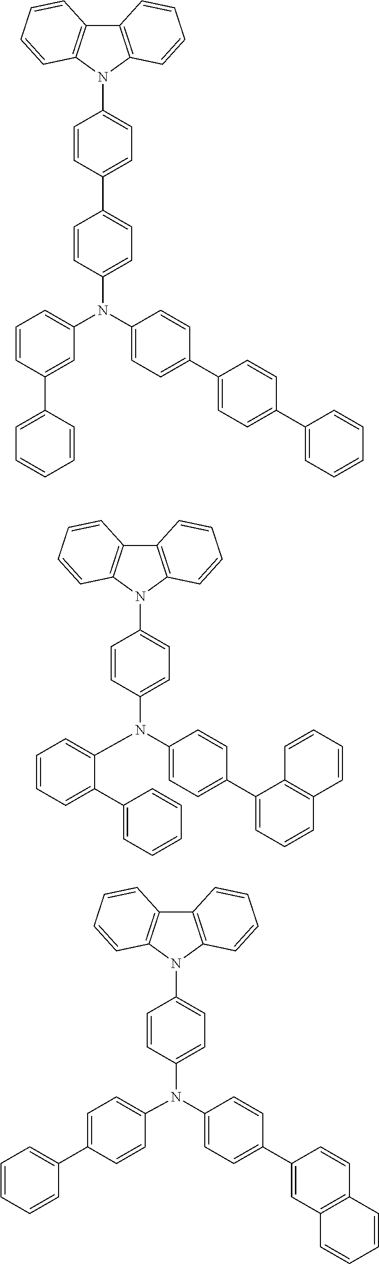 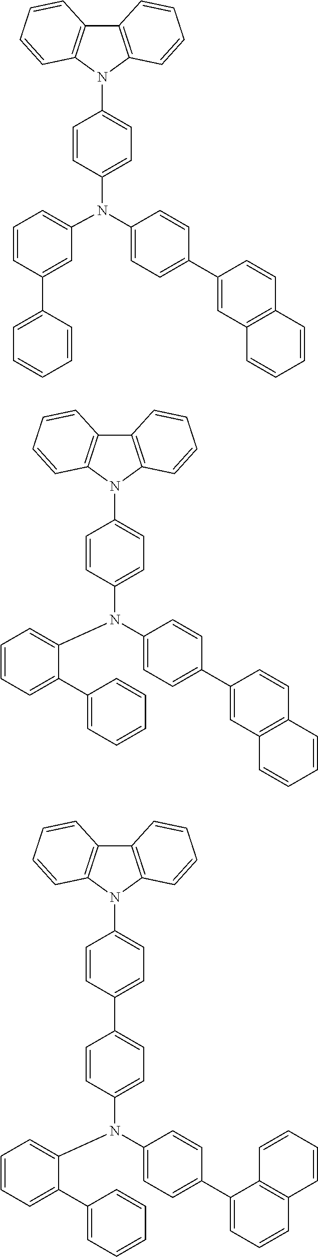 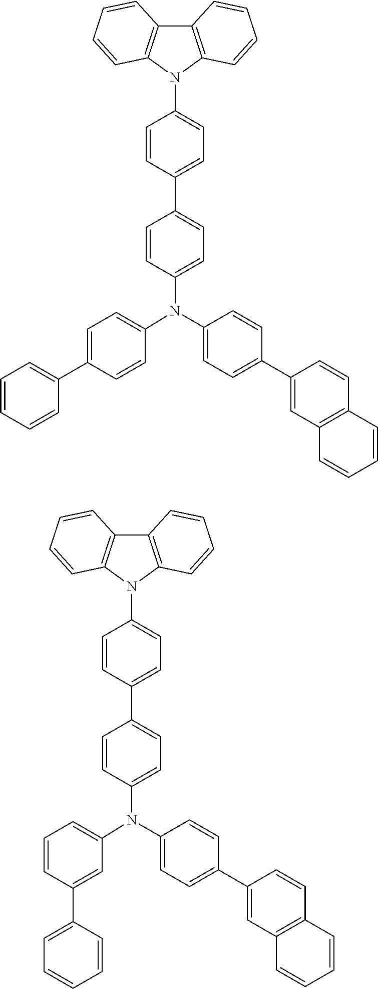 [SMILES: c1ccc(-c2ccc(N(c3ccc(-c4ccc(-n5c6ccccc6c6ccccc65)cc4)cc3)c3ccc(-c4ccc5ccccc5c4)cc3)cc2)cc1 ; c1ccc(-c2cccc(N(c3ccc(-c4ccc(-n5c6ccccc6c6ccccc65)cc4)cc3)c3ccc(-c4ccc5ccccc5c4)cc3)c2)cc1] 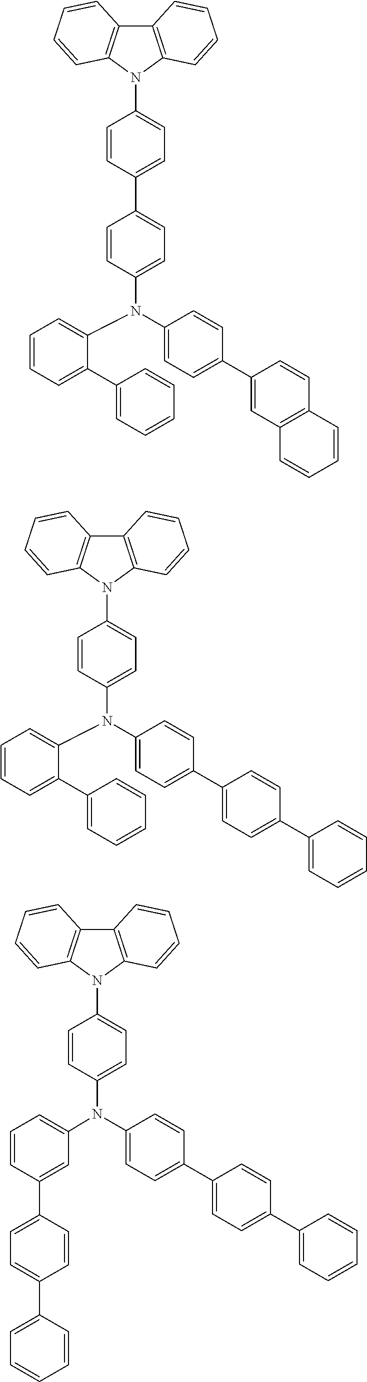 [SMILES: c1ccc(-c2ccccc2N(c2ccc(-c3ccc(-n4c5ccccc5c5ccccc54)cc3)cc2)c2ccc(-c3ccc4ccccc4c3)cc2)cc1 ; c1ccc(-c2ccc(-c3ccc(N(c4ccc(-n5c6ccccc6c6ccccc65)cc4)c4ccccc4-c4ccccc4)cc3)cc2)cc1 ; c1ccc(-c2ccc(-c3ccc(N(c4ccc(-n5c6ccccc6c6ccccc65)cc4)c4cccc(-c5ccc(-c6ccccc6)cc5)c4)cc3)cc2)cc1] 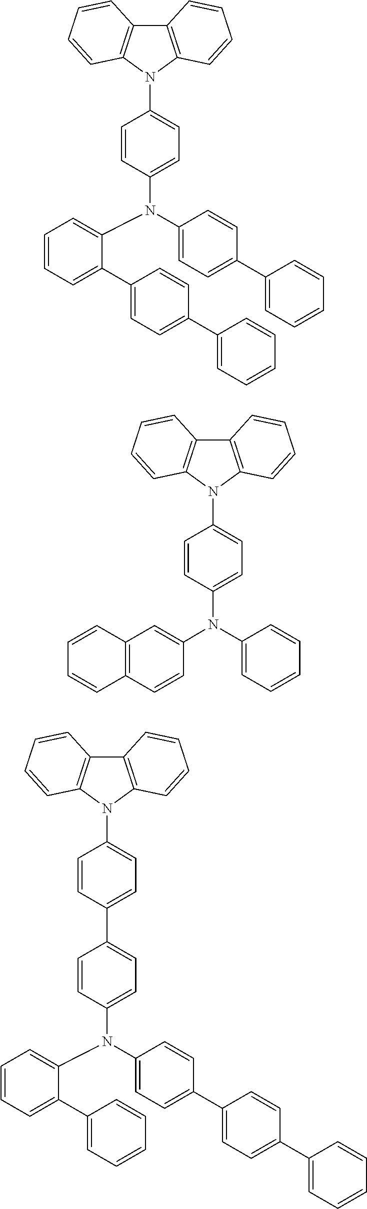 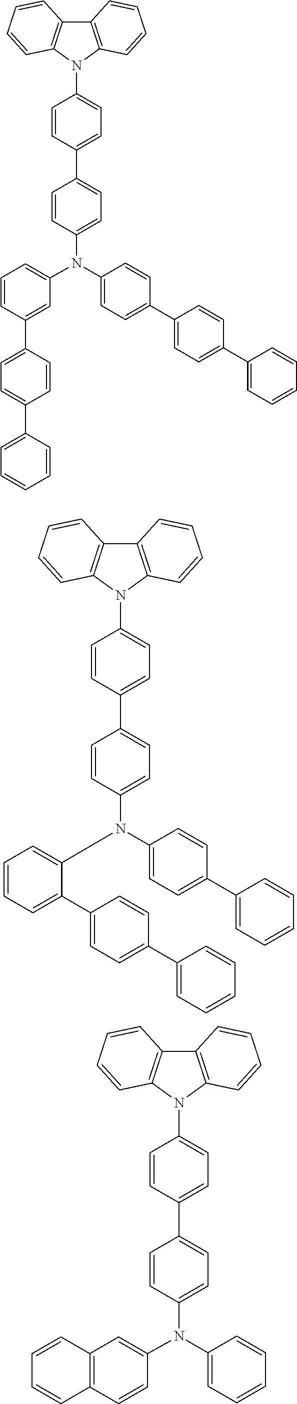 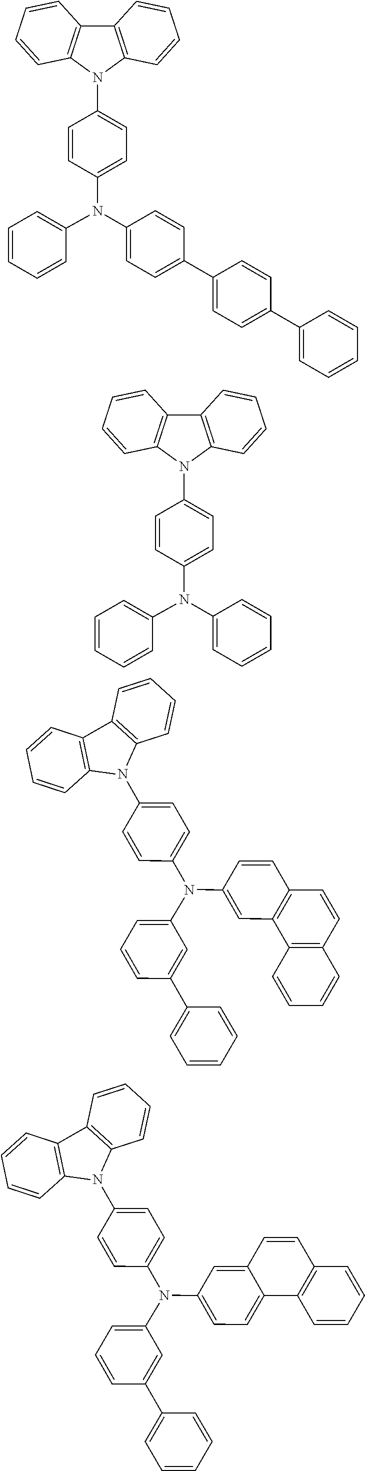 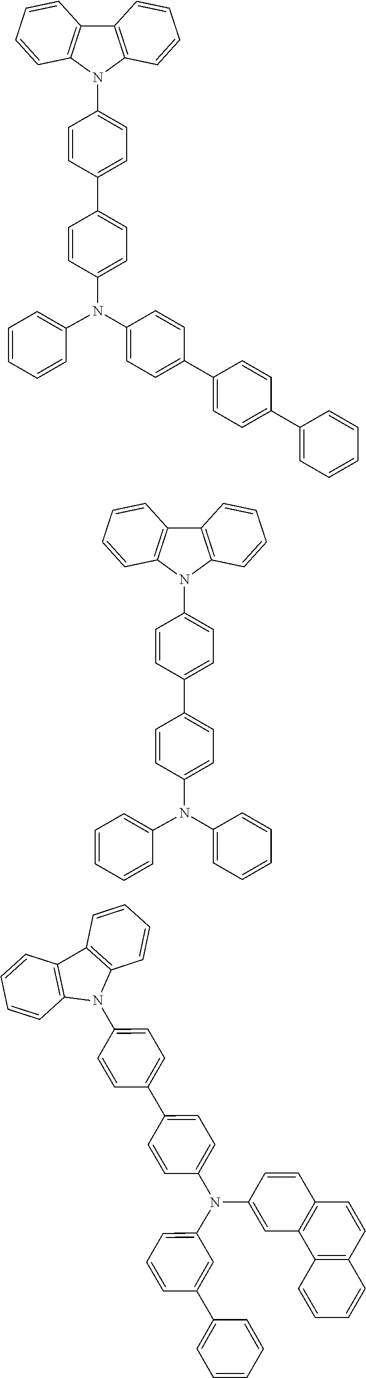 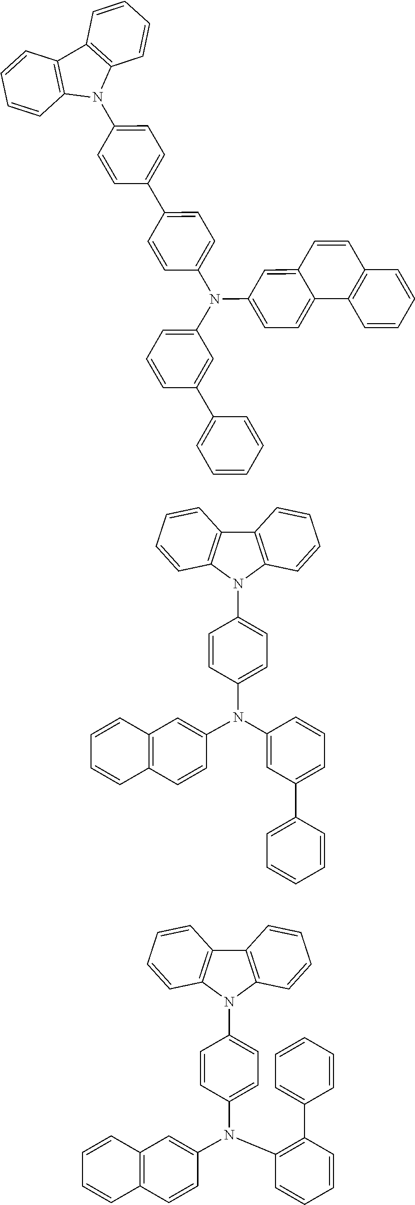 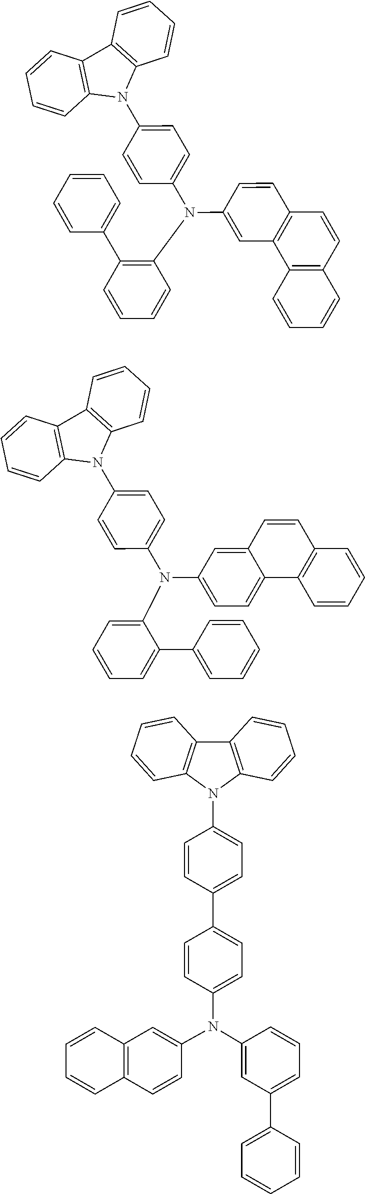 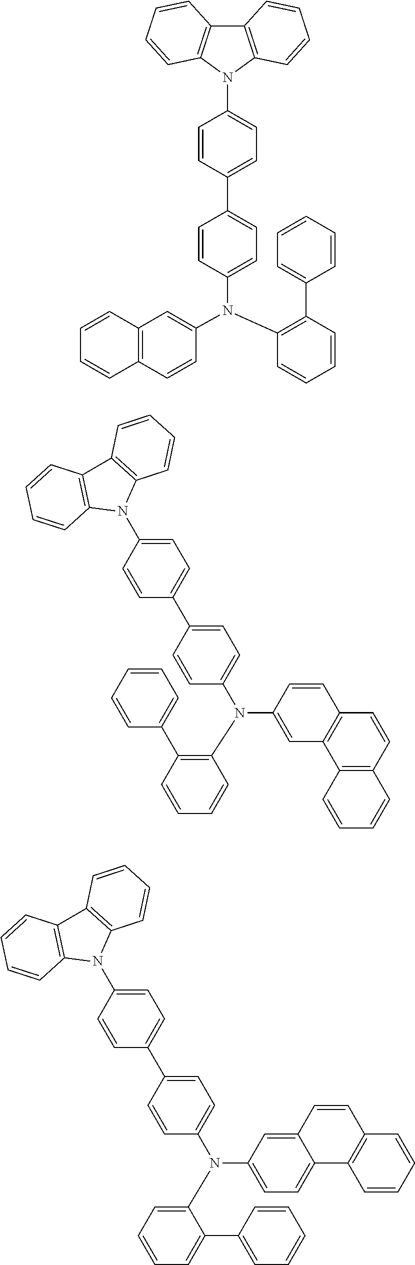 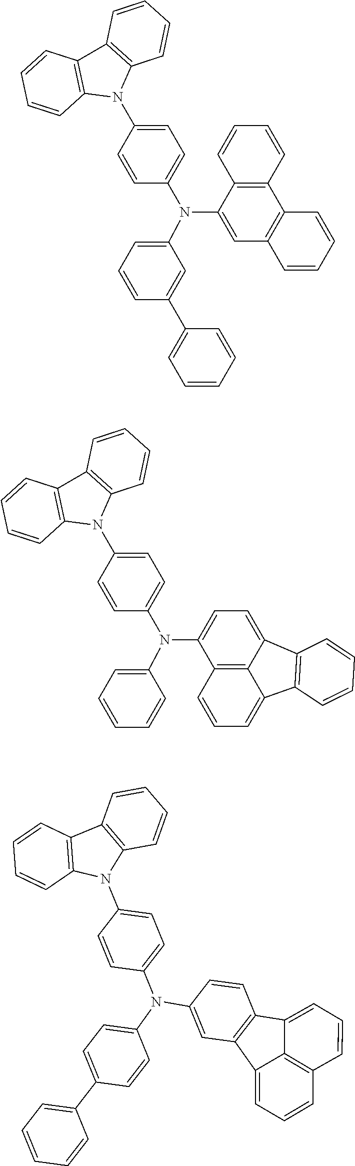 [SMILES: c1ccc(-c2cccc(N(c3ccc(-n4c5ccccc5c5ccccc54)cc3)c3cc4ccccc4c4ccccc34)c2)cc1 ; c1ccc(N(c2ccc(-n3c4ccccc4c4ccccc43)cc2)c2ccc3c4c(cccc24)-c2ccccc2-3)cc1 ; c1ccc(-c2ccc(N(c3ccc(-n4c5ccccc5c5ccccc54)cc3)c3ccc4c(c3)-c3cccc5cccc-4c35)cc2)cc1] 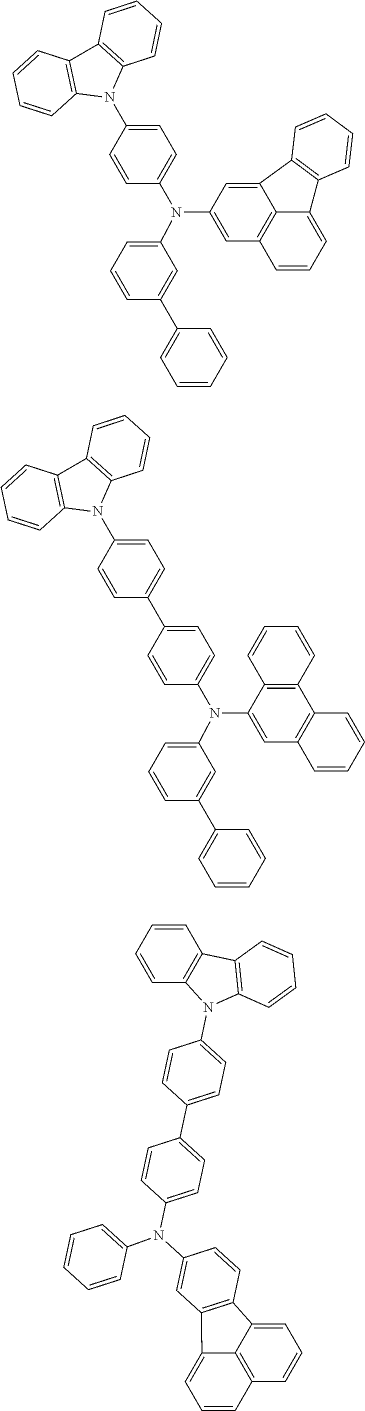 [SMILES: c1ccc(-c2cccc(N(c3ccc(-n4c5ccccc5c5ccccc54)cc3)c3cc4c5c(cccc5c3)-c3ccccc3-4)c2)cc1 ; c1ccc(-c2cccc(N(c3ccc(-c4ccc(-n5c6ccccc6c6ccccc65)cc4)cc3)c3cc4ccccc4c4ccccc34)c2)cc1 ; c1ccc(N(c2ccc(-c3ccc(-n4c5ccccc5c5ccccc54)cc3)cc2)c2ccc3c(c2)-c2cccc4cccc-3c24)cc1] 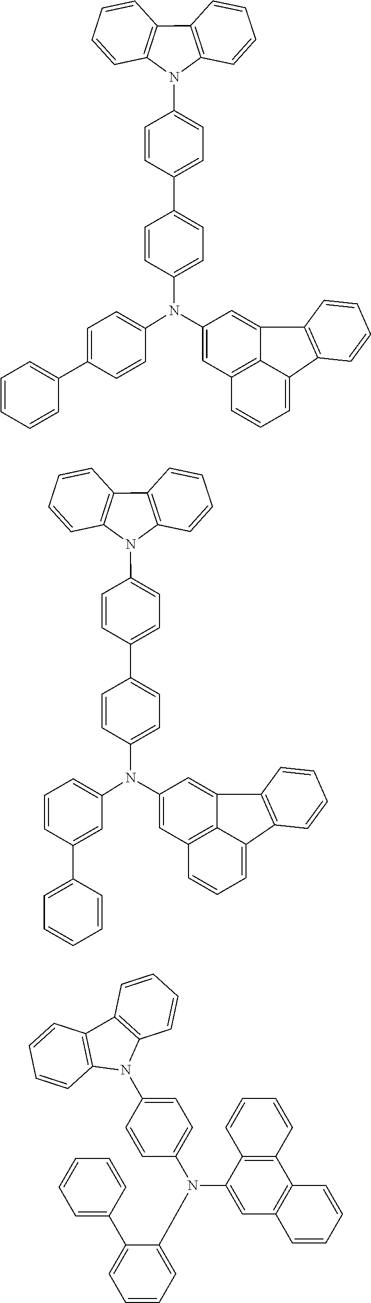 [SMILES: c1ccc(-c2ccc(N(c3ccc(-c4ccc(-n5c6ccccc6c6ccccc65)cc4)cc3)c3cc4c5c(cccc5c3)-c3ccccc3-4)cc2)cc1 ; c1ccc(-c2cccc(N(c3ccc(-c4ccc(-n5c6ccccc6c6ccccc65)cc4)cc3)c3cc4c5c(cccc5c3)-c3ccccc3-4)c2)cc1 ; c1ccc(-c2ccccc2N(c2ccc(-n3c4ccccc4c4ccccc43)cc2)c2cc3ccccc3c3ccccc23)cc1] 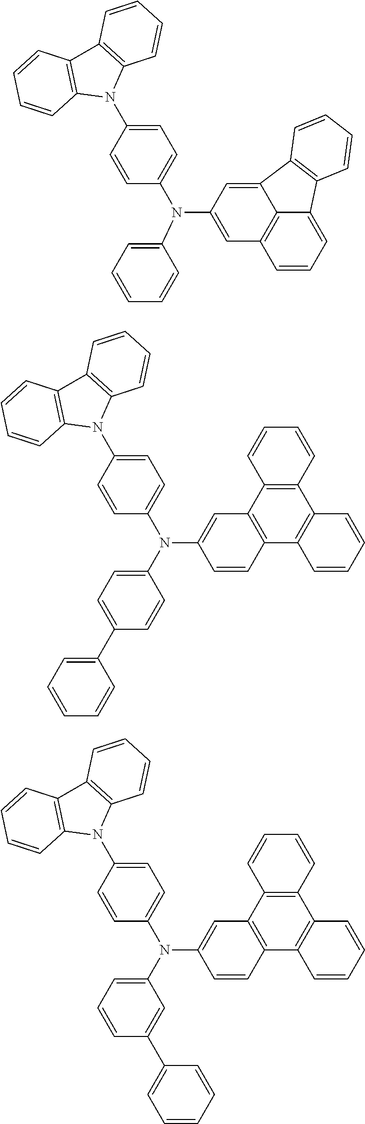 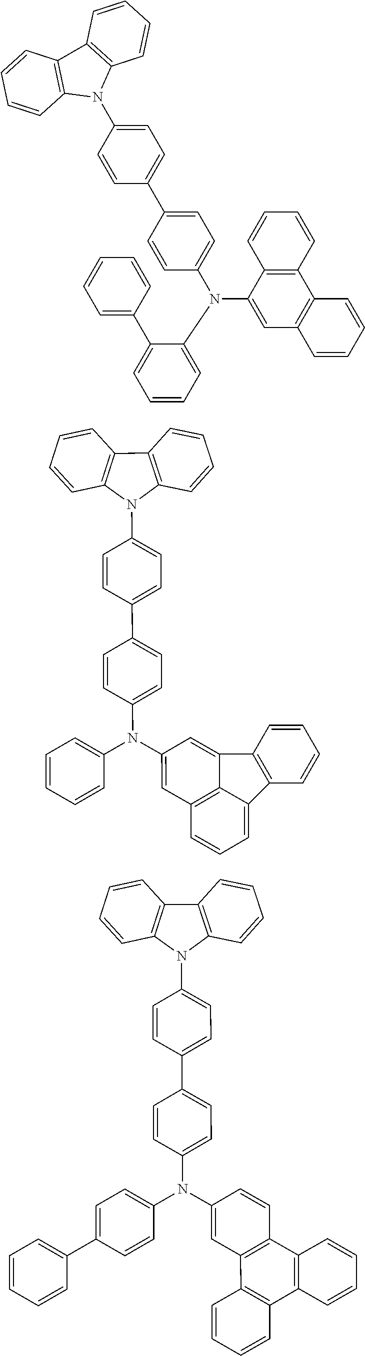 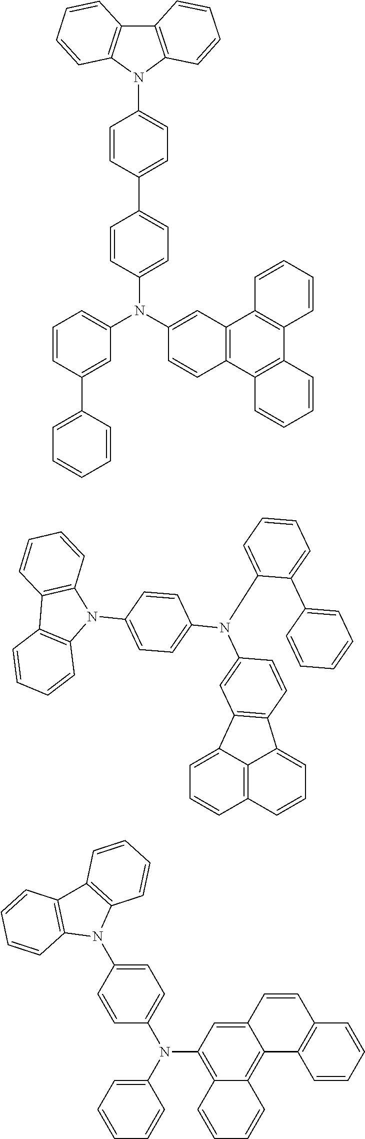 [SMILES: c1ccc(-c2cccc(N(c3ccc(-c4ccc(-n5c6ccccc6c6ccccc65)cc4)cc3)c3ccc4c5ccccc5c5ccccc5c4c3)c2)cc1 ; c1ccc(-c2ccccc2N(c2ccc(-n3c4ccccc4c4ccccc43)cc2)c2ccc3c(c2)-c2cccc4cccc-3c24)cc1 ; c1ccc(N(c2ccc(-n3c4ccccc4c4ccccc43)cc2)c2cc3ccc4ccccc4c3c3ccccc23)cc1] 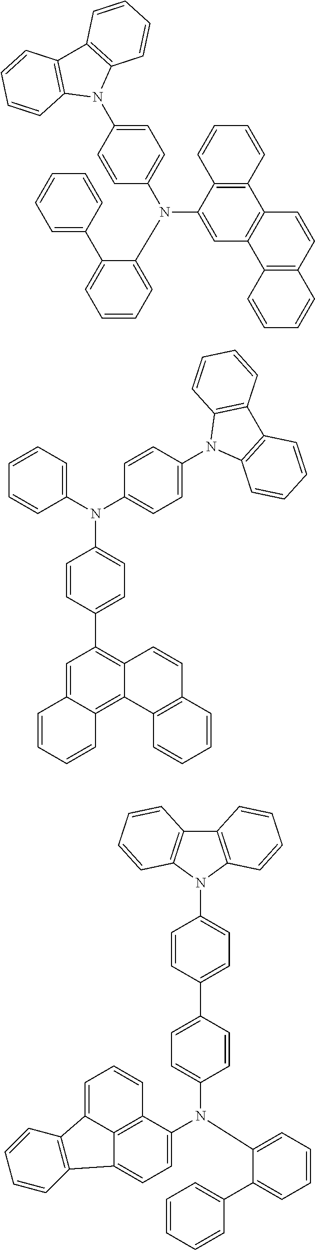 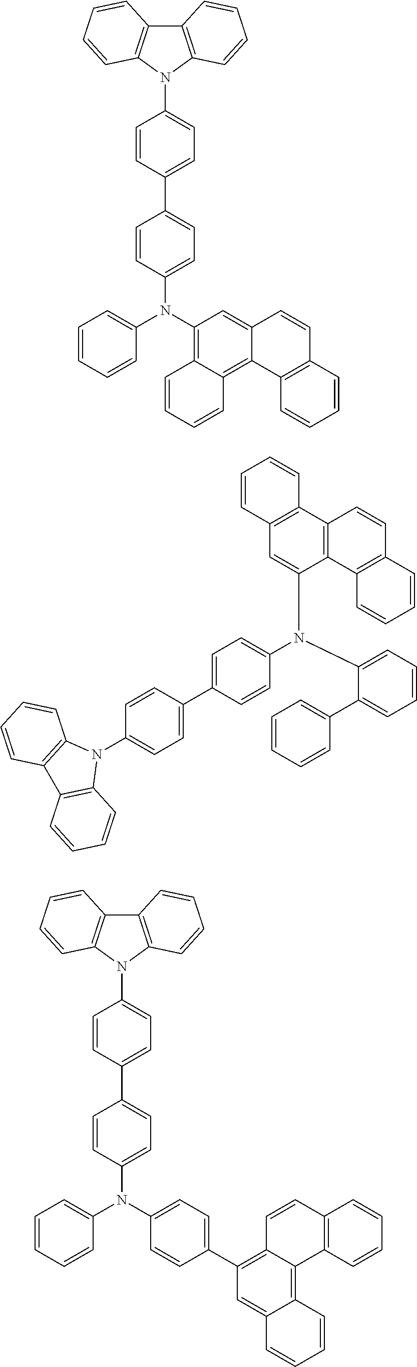 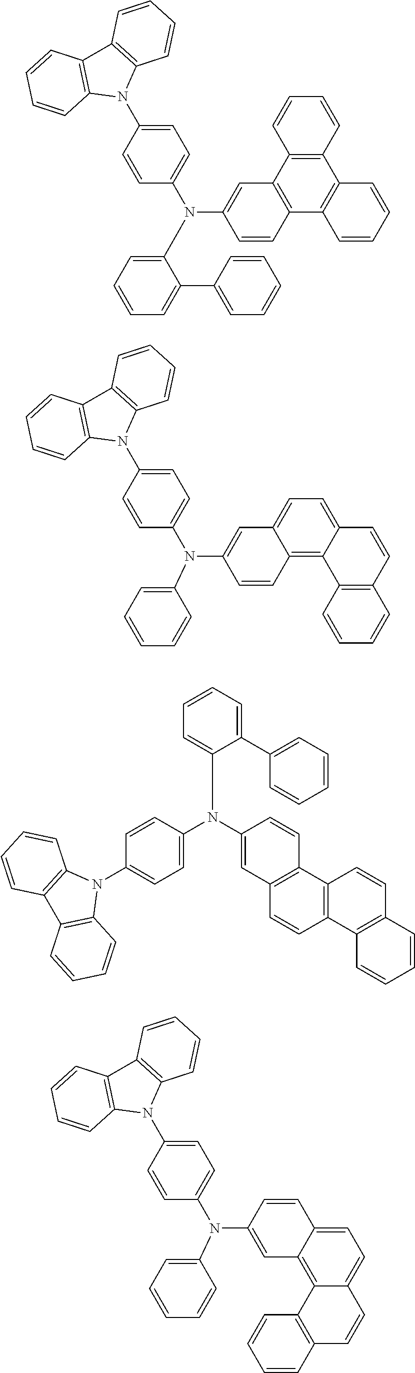 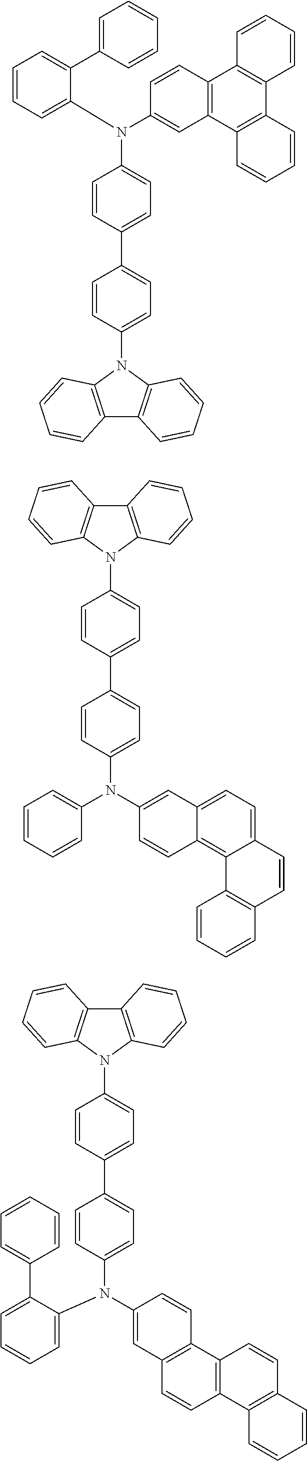 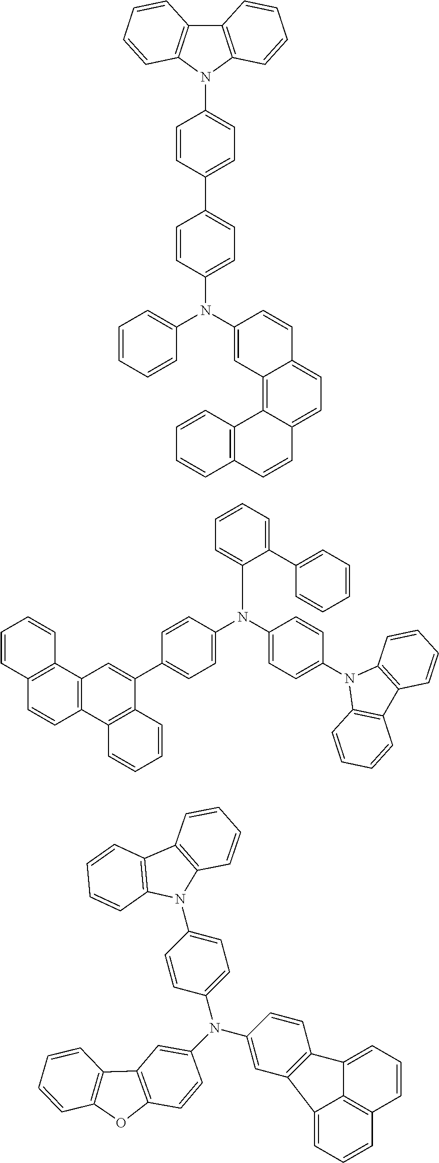 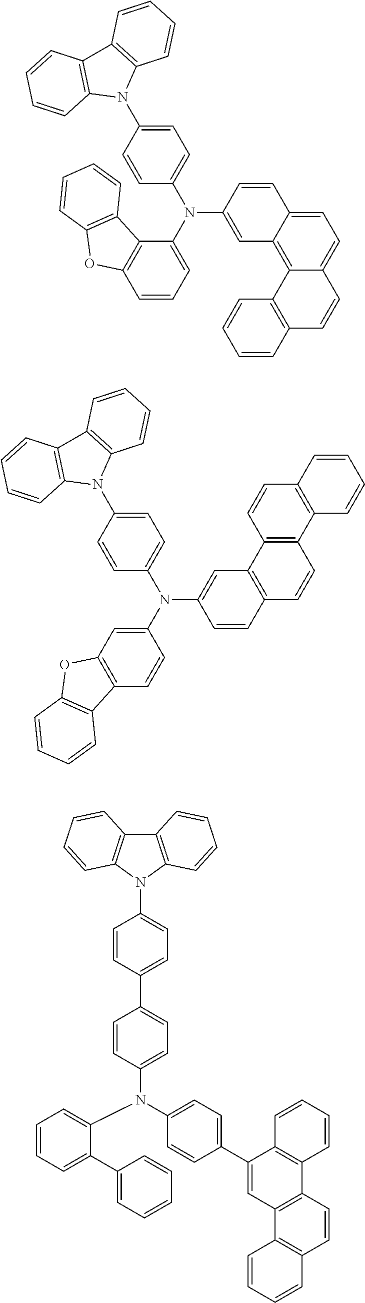 [SMILES: c1ccc2c(c1)ccc1ccc3ccc(N(c4ccc(-n5c6ccccc6c6ccccc65)cc4)c4cccc5oc6ccccc6c45)cc3c12 ; c1ccc2c(c1)ccc1c3cc(N(c4ccc(-n5c6ccccc6c6ccccc65)cc4)c4ccc5c(c4)oc4ccccc45)ccc3ccc21 ; c1ccc(-c2ccccc2N(c2ccc(-c3ccc(-n4c5ccccc5c5ccccc54)cc3)cc2)c2ccc(-c3cc4c5ccccc5ccc4c4ccccc34)cc2)cc1] 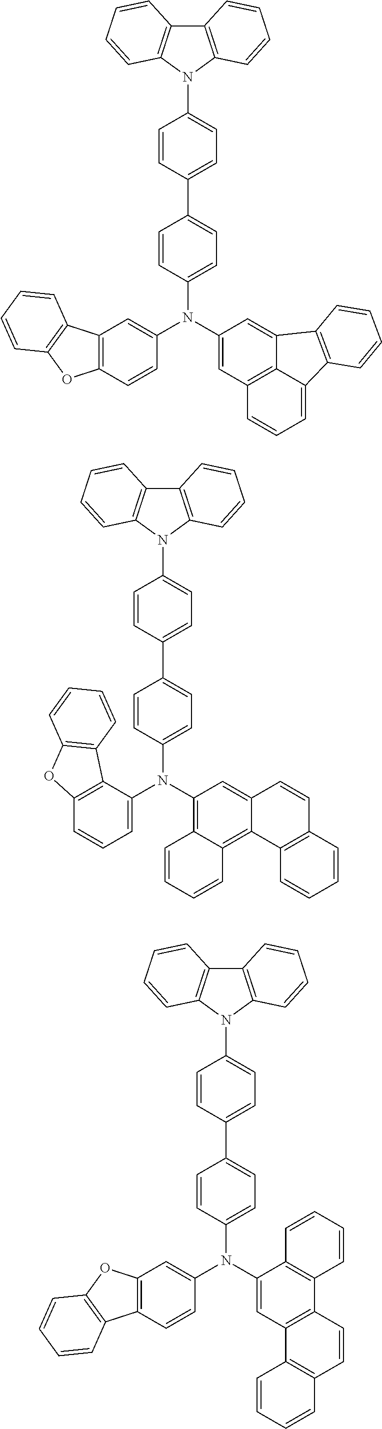 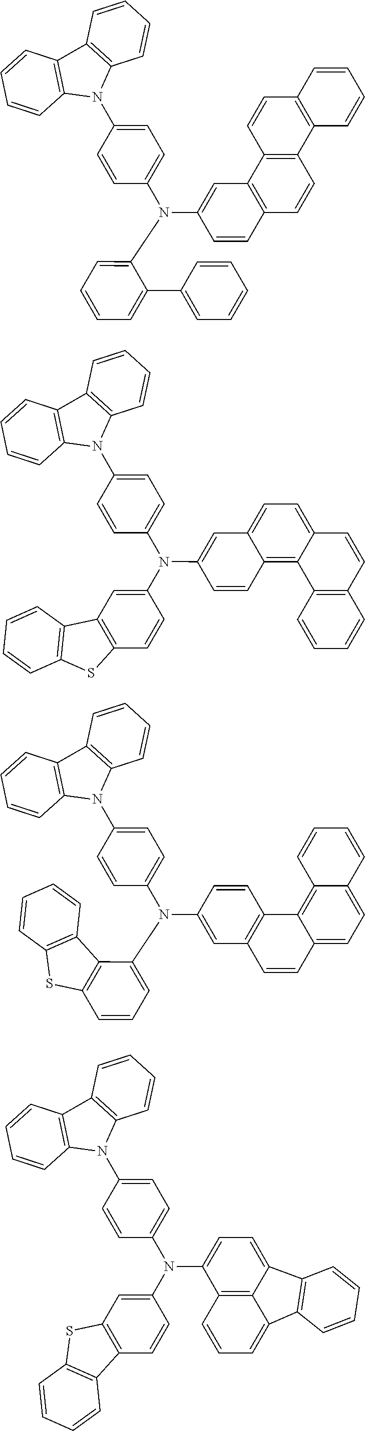 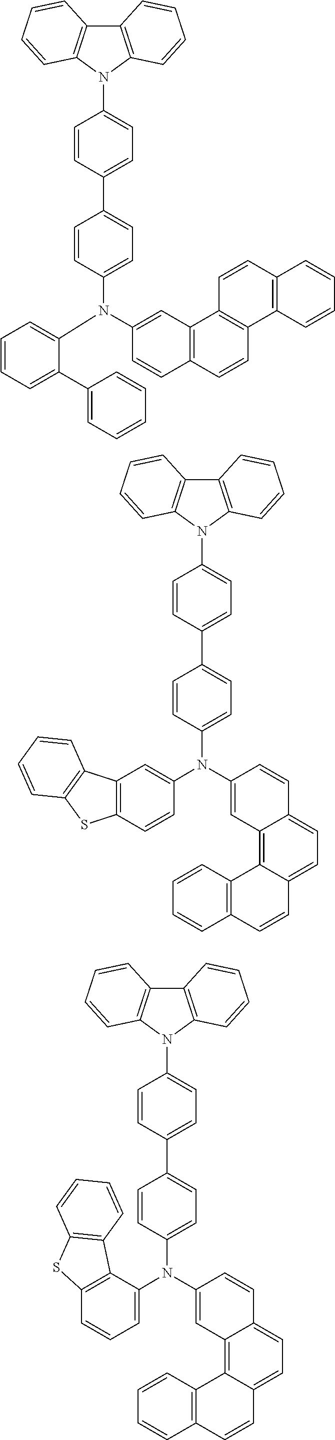 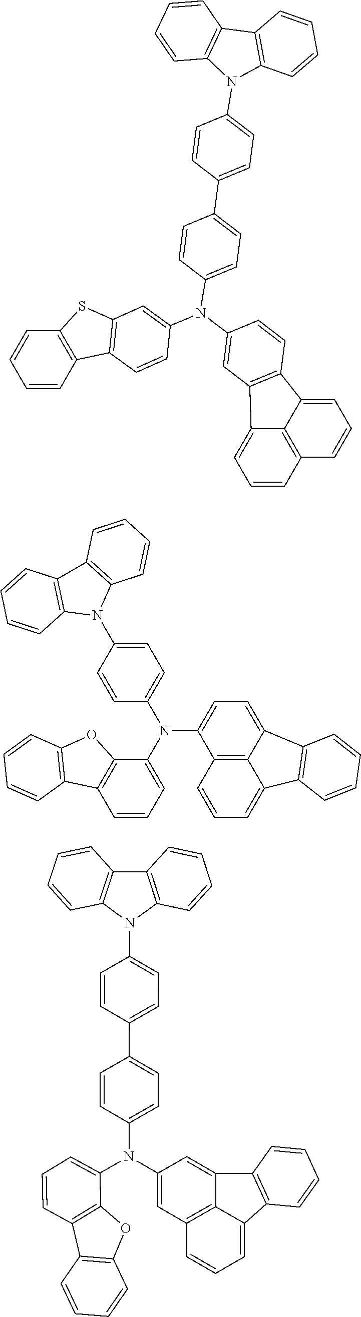 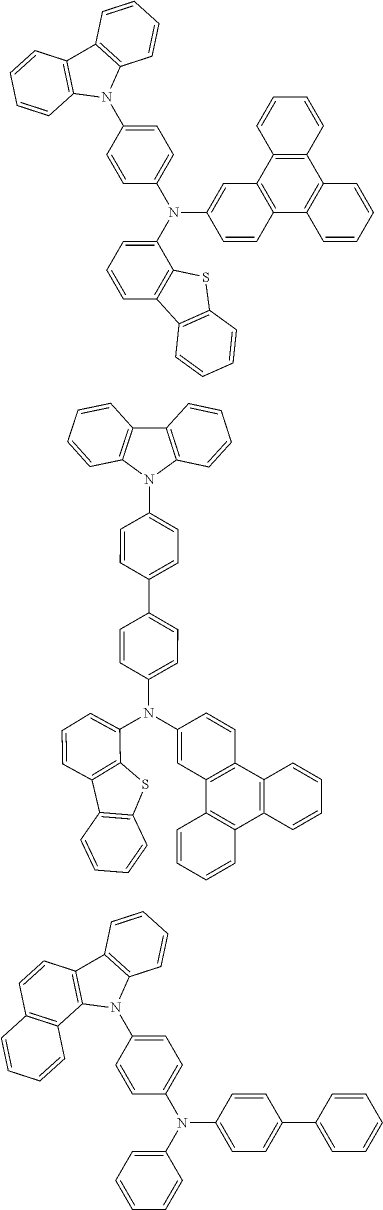 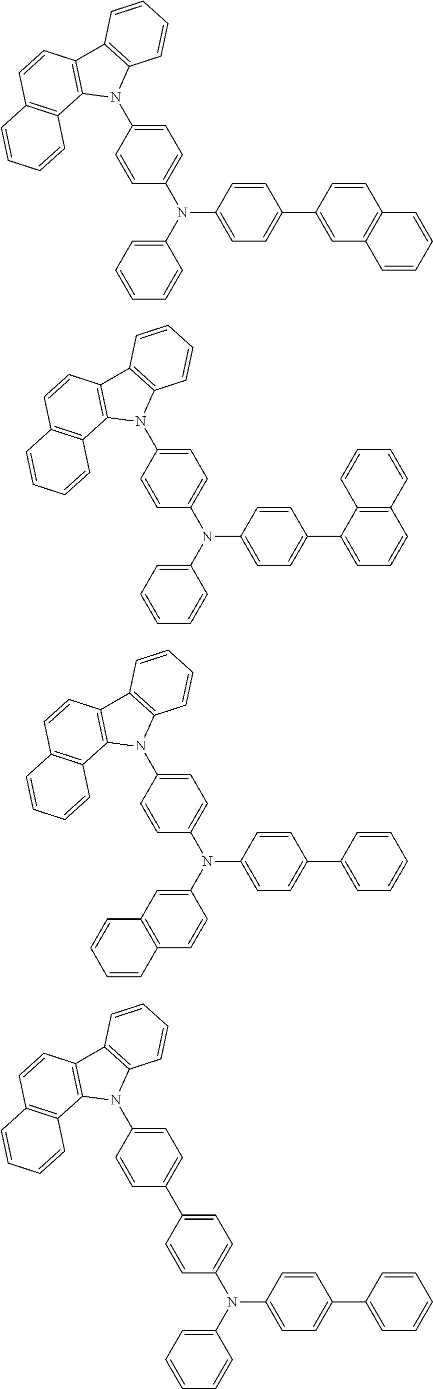 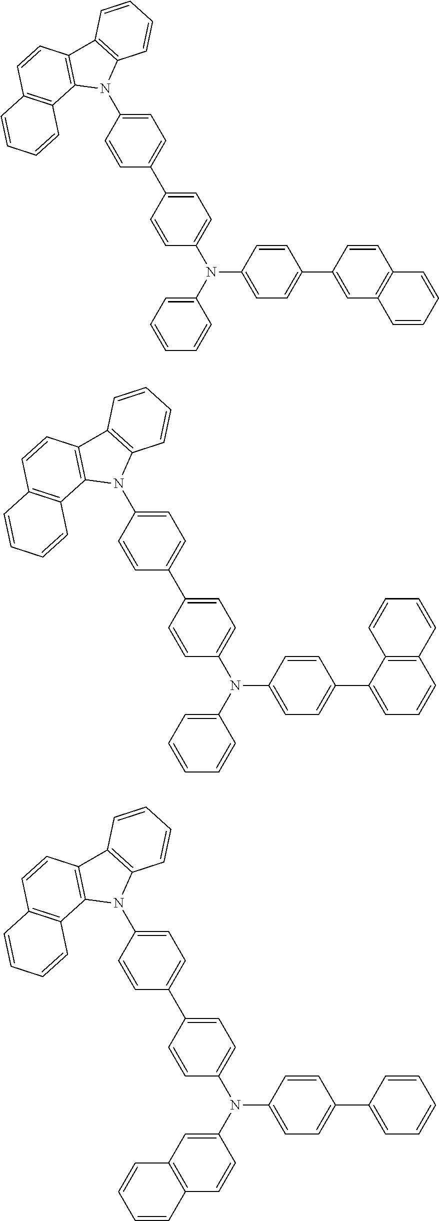 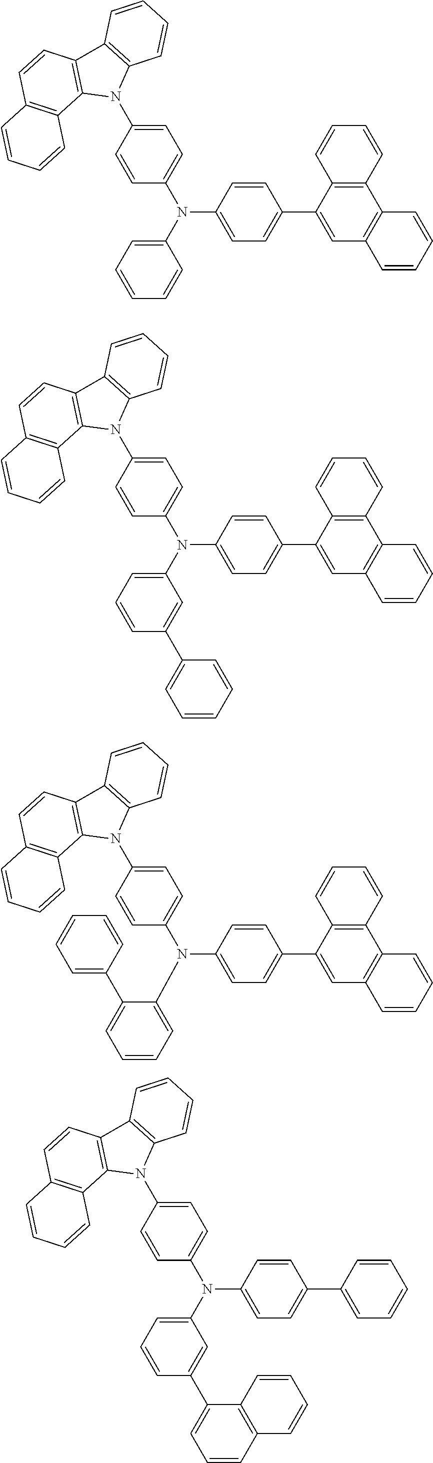 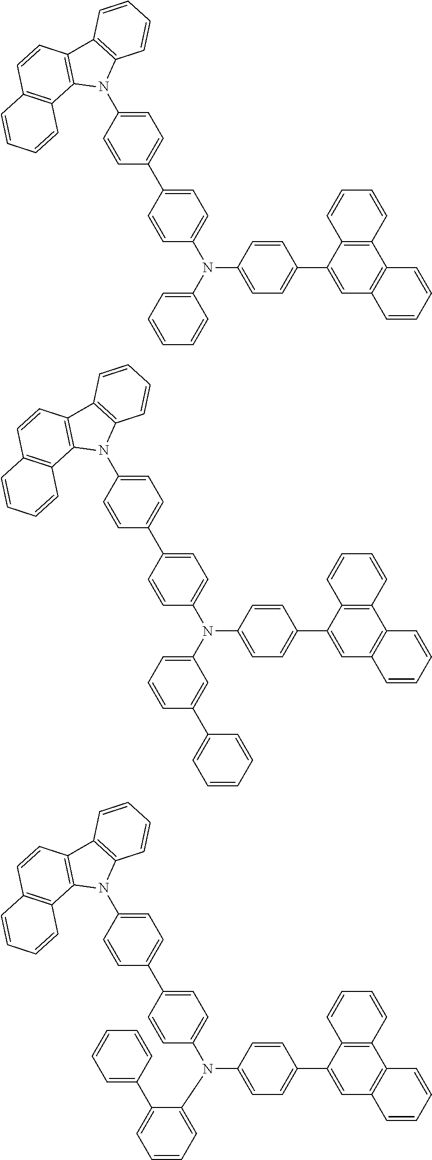 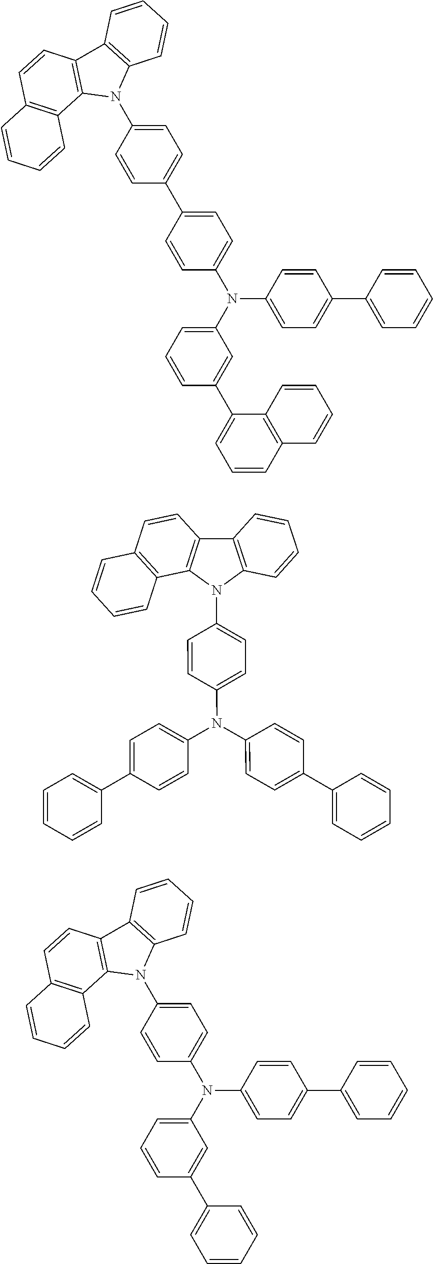 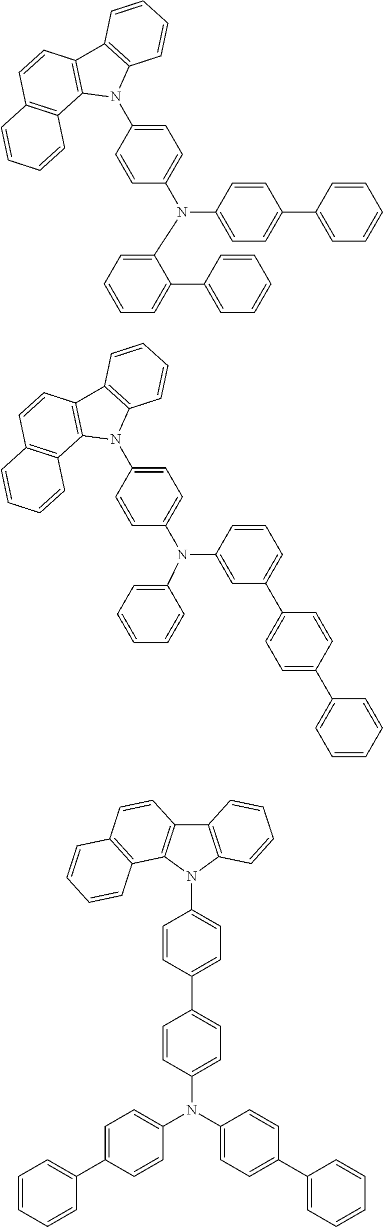 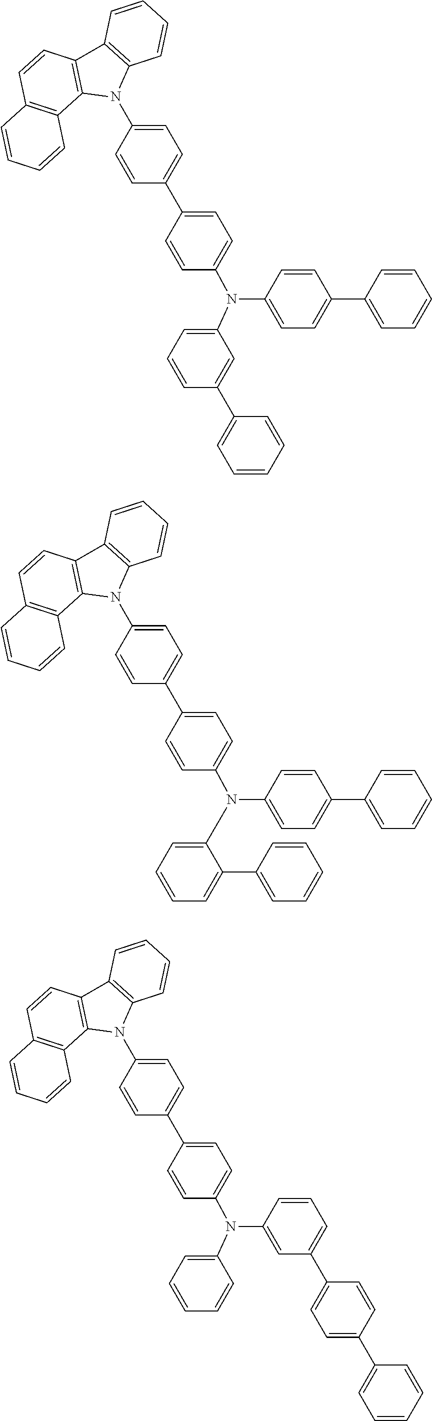 [SMILES: c1ccc(-c2ccc(N(c3ccc(-c4ccc(-n5c6ccccc6c6ccc7ccccc7c65)cc4)cc3)c3cccc(-c4ccccc4)c3)cc2)cc1 ; c1ccc(-c2ccc(N(c3ccc(-c4ccc(-n5c6ccccc6c6ccc7ccccc7c65)cc4)cc3)c3ccccc3-c3ccccc3)cc2)cc1 ; c1ccc(-c2ccc(-c3cccc(N(c4ccccc4)c4ccc(-c5ccc(-n6c7ccccc7c7ccc8ccccc8c76)cc5)cc4)c3)cc2)cc1] 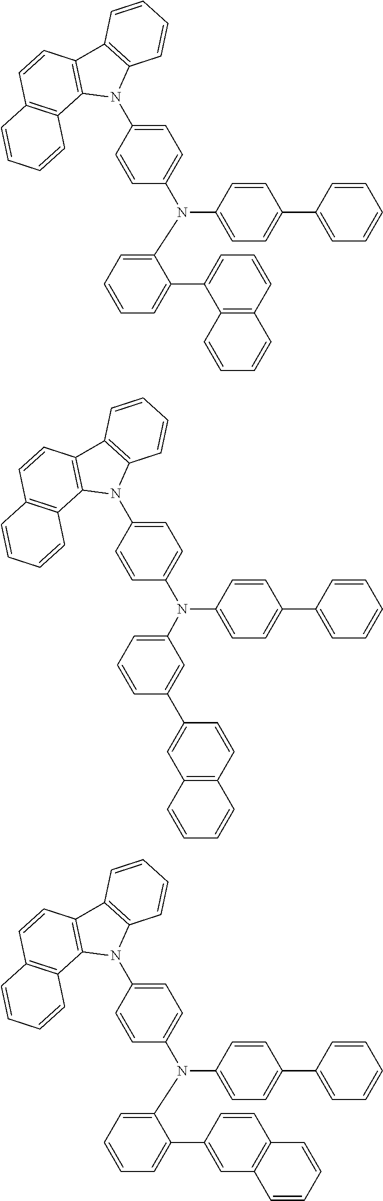 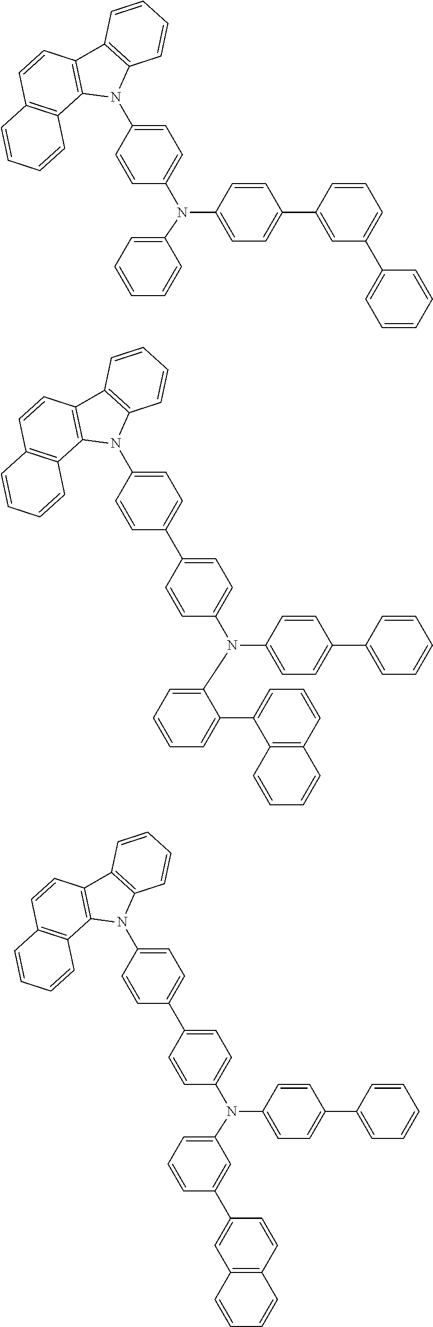 [SMILES: c1ccc(-c2cccc(-c3ccc(N(c4ccccc4)c4ccc(-n5c6ccccc6c6ccc7ccccc7c65)cc4)cc3)c2)cc1 ; c1ccc(-c2ccc(N(c3ccc(-c4ccc(-n5c6ccccc6c6ccc7ccccc7c65)cc4)cc3)c3ccccc3-c3cccc4ccccc34)cc2)cc1 ; c1ccc(-c2ccc(N(c3ccc(-c4ccc(-n5c6ccccc6c6ccc7ccccc7c65)cc4)cc3)c3cccc(-c4ccc5ccccc5c4)c3)cc2)cc1] 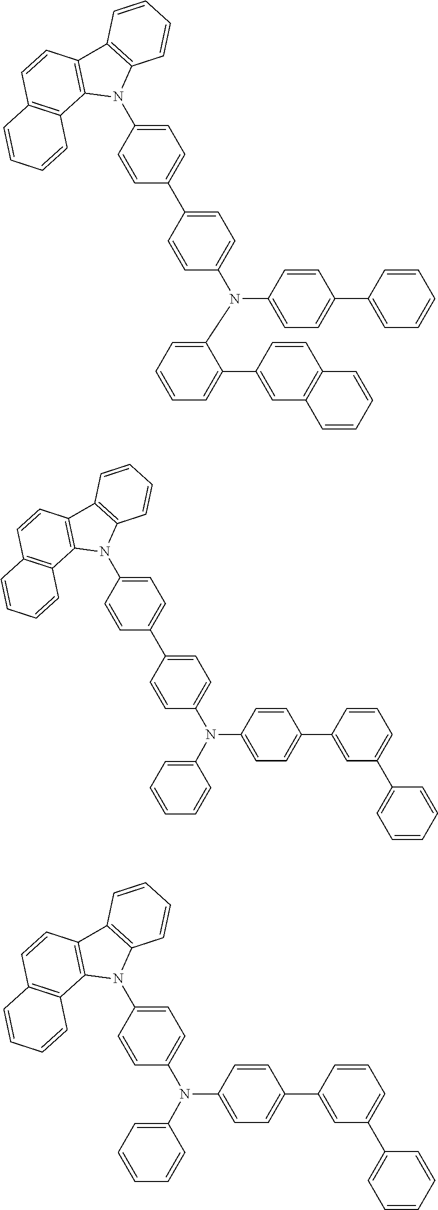 [SMILES: c1ccc(-c2ccc(N(c3ccc(-c4ccc(-n5c6ccccc6c6ccc7ccccc7c65)cc4)cc3)c3ccccc3-c3ccc4ccccc4c3)cc2)cc1 ; c1ccc(-c2cccc(-c3ccc(N(c4ccccc4)c4ccc(-c5ccc(-n6c7ccccc7c7ccc8ccccc8c76)cc5)cc4)cc3)c2)cc1 ; c1ccc(-c2cccc(-c3ccc(N(c4ccccc4)c4ccc(-n5c6ccccc6c6ccc7ccccc7c65)cc4)cc3)c2)cc1] 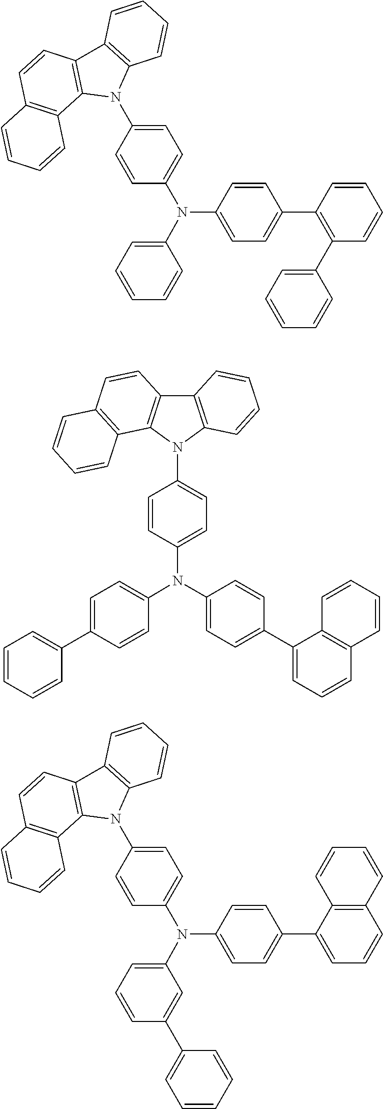 [SMILES: c1ccc(-c2ccccc2-c2ccc(N(c3ccccc3)c3ccc(-n4c5ccccc5c5ccc6ccccc6c54)cc3)cc2)cc1 ; c1ccc(-c2ccc(N(c3ccc(-c4cccc5ccccc45)cc3)c3ccc(-n4c5ccccc5c5ccc6ccccc6c54)cc3)cc2)cc1 ; c1ccc(-c2cccc(N(c3ccc(-c4cccc5ccccc45)cc3)c3ccc(-n4c5ccccc5c5ccc6ccccc6c54)cc3)c2)cc1] 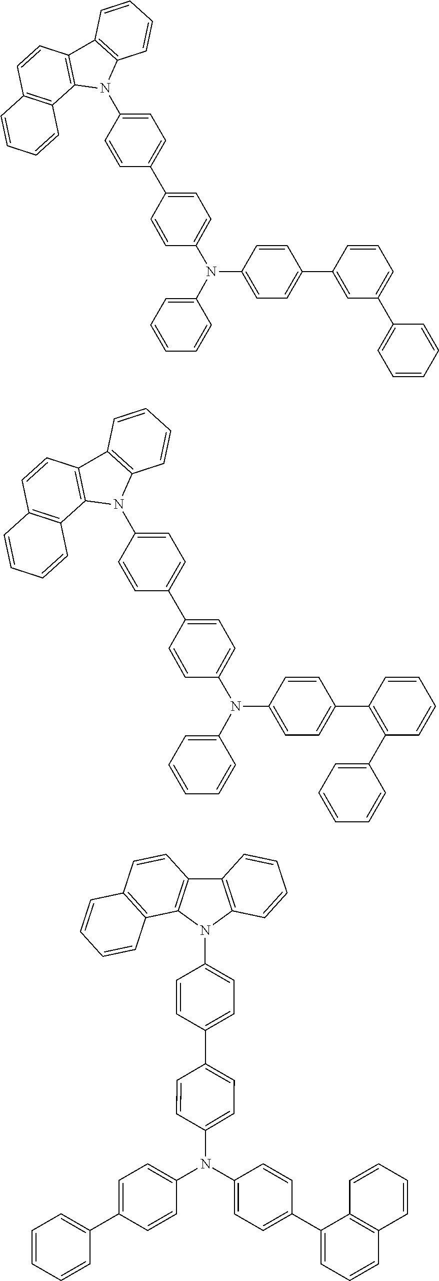 [SMILES: c1ccc(-c2cccc(-c3ccc(N(c4ccccc4)c4ccc(-c5ccc(-n6c7ccccc7c7ccc8ccccc8c76)cc5)cc4)cc3)c2)cc1 ; c1ccc(-c2ccccc2-c2ccc(N(c3ccccc3)c3ccc(-c4ccc(-n5c6ccccc6c6ccc7ccccc7c65)cc4)cc3)cc2)cc1 ; c1ccc(-c2ccc(N(c3ccc(-c4ccc(-n5c6ccccc6c6ccc7ccccc7c65)cc4)cc3)c3ccc(-c4cccc5ccccc45)cc3)cc2)cc1] 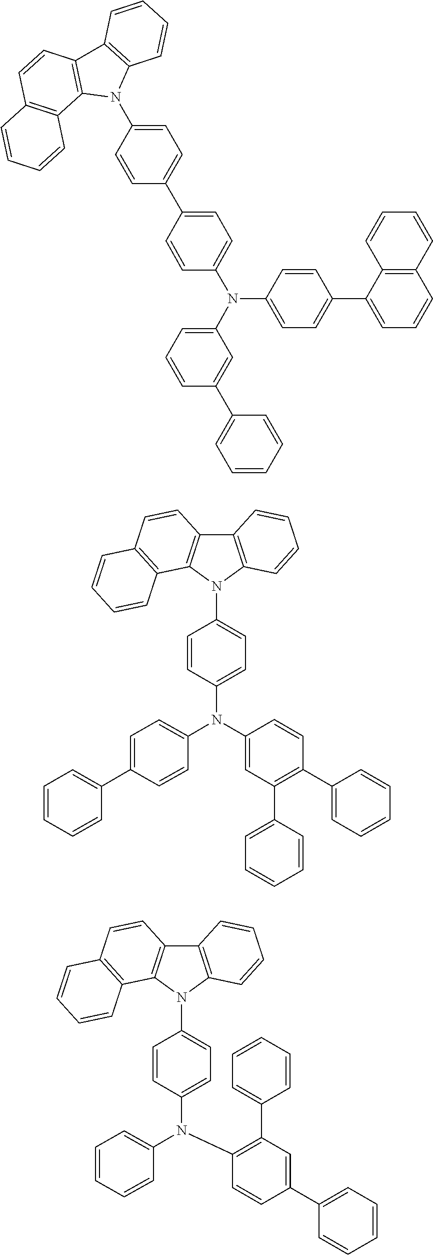 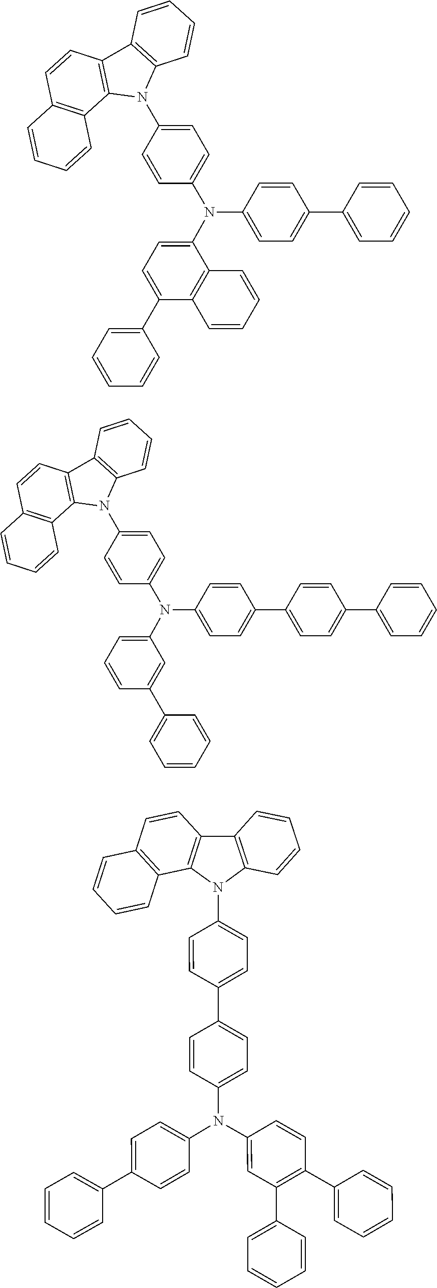 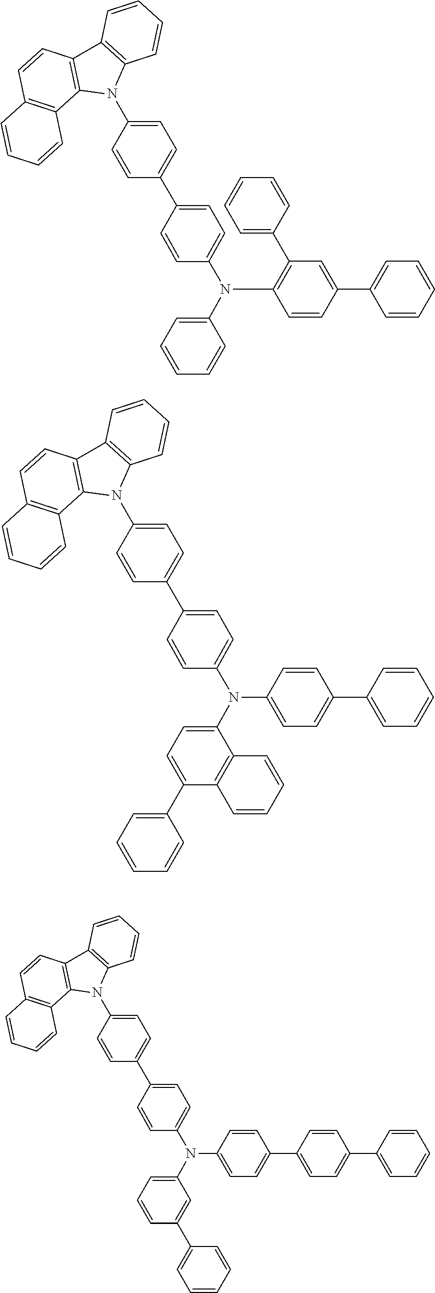 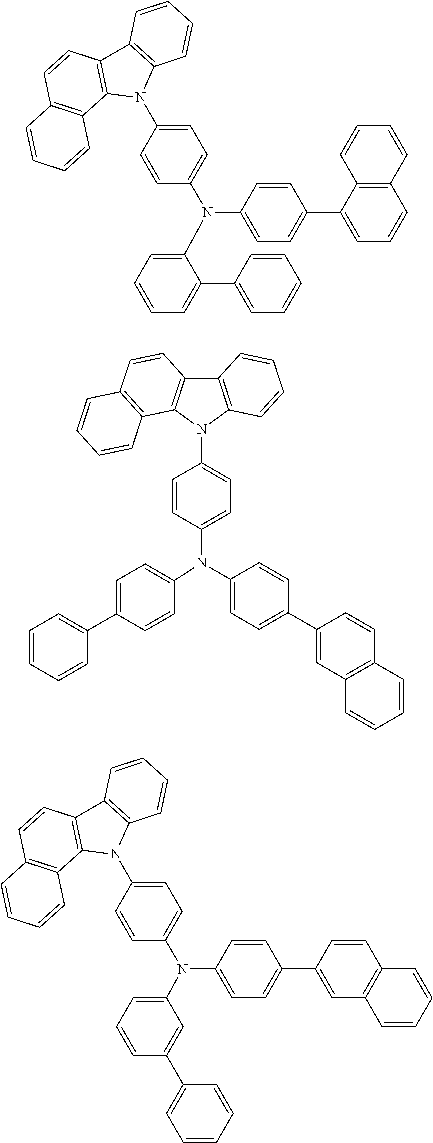 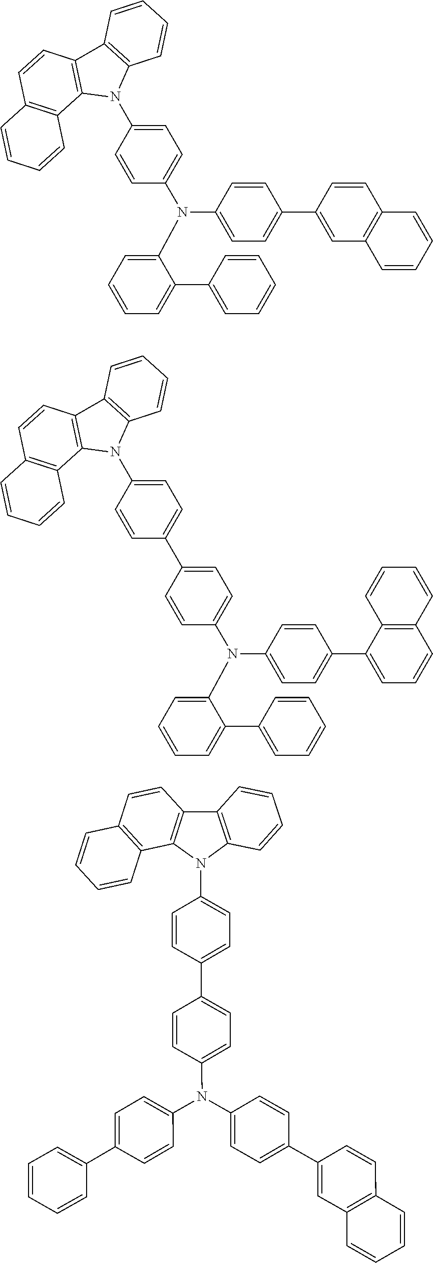 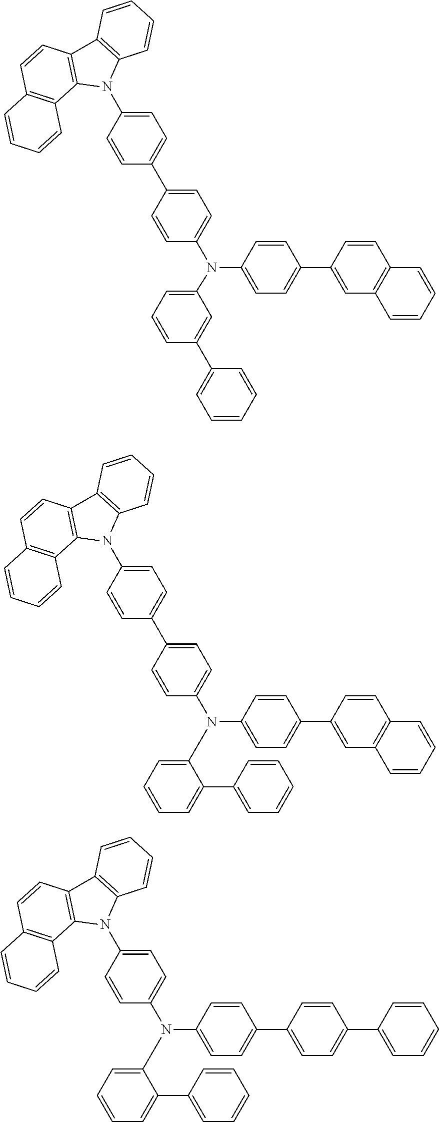 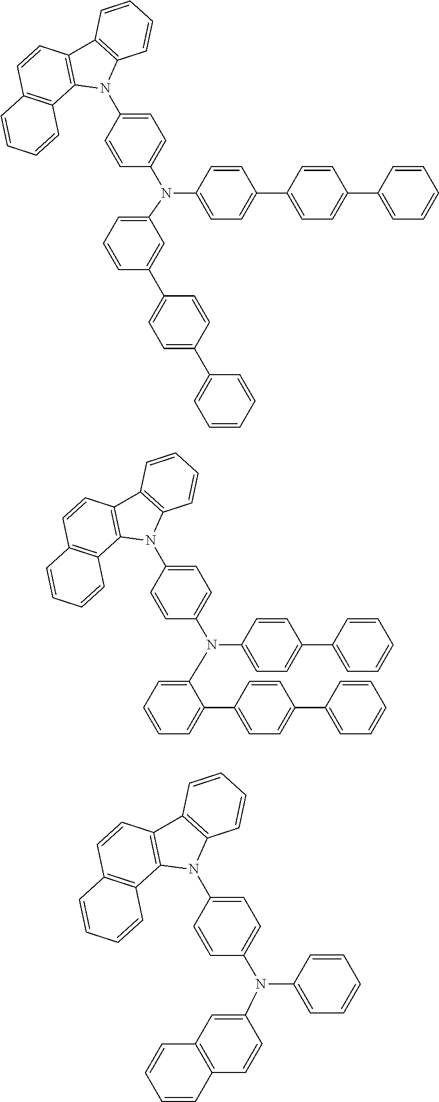 [SMILES: c1ccc(-c2ccc(-c3ccc(N(c4ccc(-n5c6ccccc6c6ccc7ccccc7c65)cc4)c4cccc(-c5ccc(-c6ccccc6)cc5)c4)cc3)cc2)cc1 ; c1ccc(-c2ccc(-c3ccccc3N(c3ccc(-c4ccccc4)cc3)c3ccc(-n4c5ccccc5c5ccc6ccccc6c54)cc3)cc2)cc1 ; c1ccc(N(c2ccc(-n3c4ccccc4c4ccc5ccccc5c43)cc2)c2ccc3ccccc3c2)cc1] 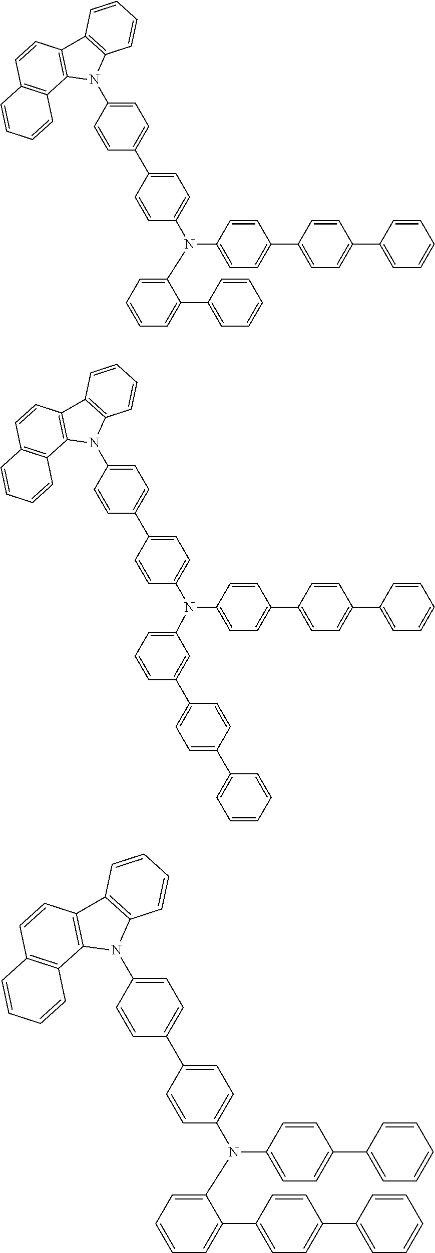 [SMILES: c1ccc(-c2ccc(-c3ccc(N(c4ccc(-c5ccc(-n6c7ccccc7c7ccc8ccccc8c76)cc5)cc4)c4ccccc4-c4ccccc4)cc3)cc2)cc1 ; c1ccc(-c2ccc(-c3ccc(N(c4ccc(-c5ccc(-n6c7ccccc7c7ccc8ccccc8c76)cc5)cc4)c4cccc(-c5ccc(-c6ccccc6)cc5)c4)cc3)cc2)cc1 ; c1ccc(-c2ccc(-c3ccccc3N(c3ccc(-c4ccccc4)cc3)c3ccc(-c4ccc(-n5c6ccccc6c6ccc7ccccc7c65)cc4)cc3)cc2)cc1] 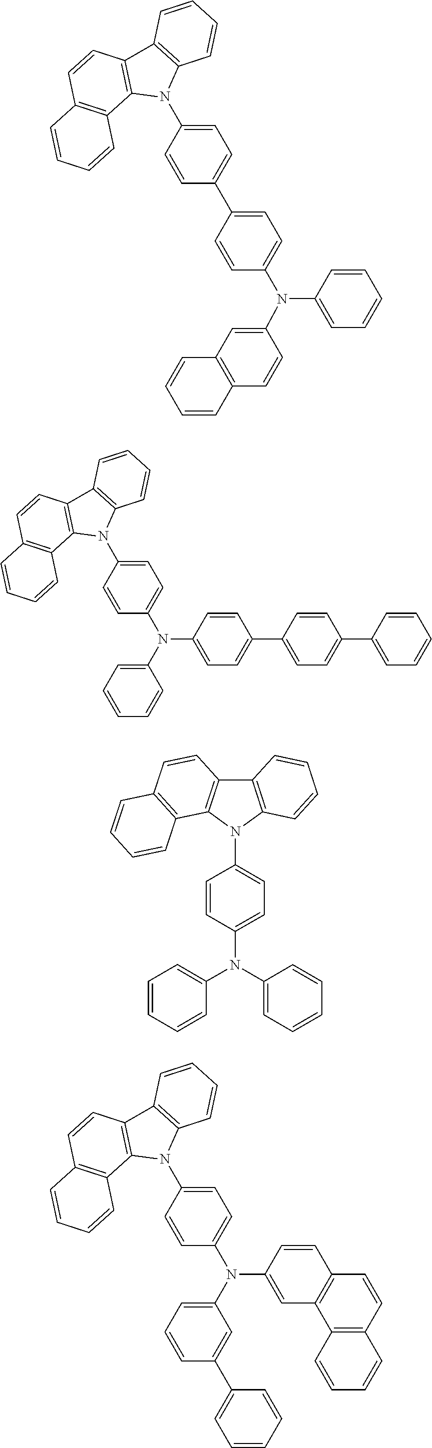 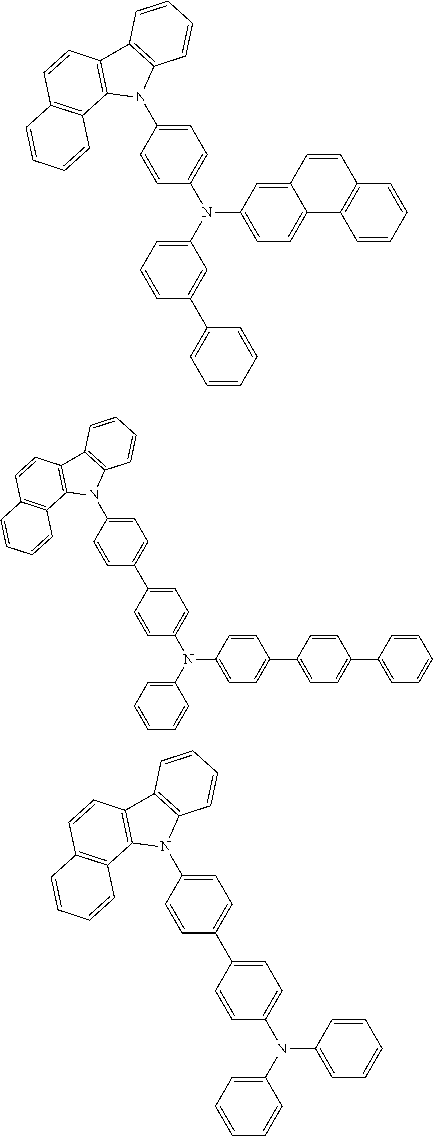 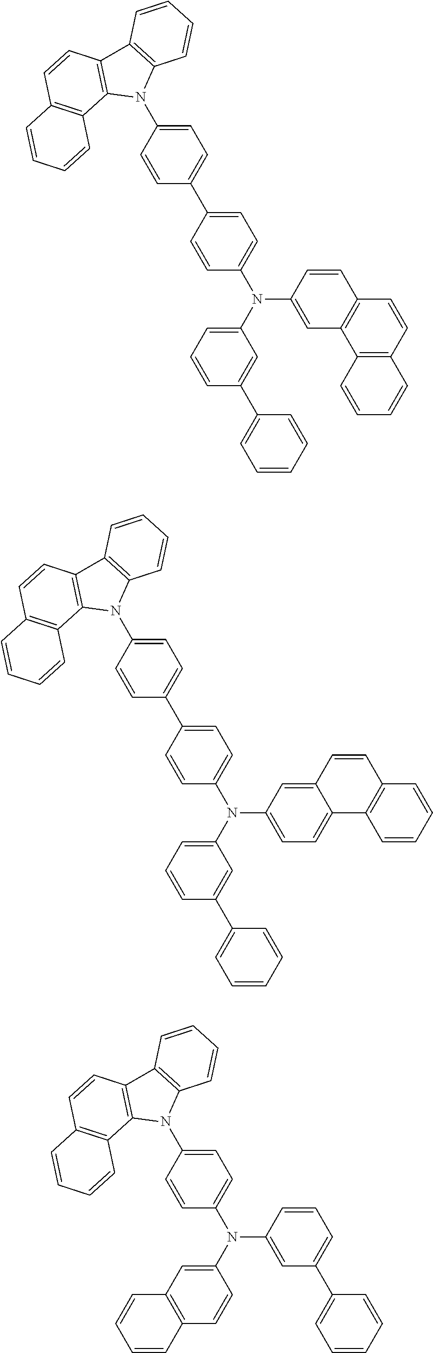 [SMILES: c1ccc(-c2cccc(N(c3ccc(-c4ccc(-n5c6ccccc6c6ccc7ccccc7c65)cc4)cc3)c3ccc4ccc5ccccc5c4c3)c2)cc1 ; c1ccc(-c2cccc(N(c3ccc(-c4ccc(-n5c6ccccc6c6ccc7ccccc7c65)cc4)cc3)c3ccc4c(ccc5ccccc54)c3)c2)cc1 ; c1ccc(-c2cccc(N(c3ccc(-n4c5ccccc5c5ccc6ccccc6c54)cc3)c3ccc4ccccc4c3)c2)cc1] 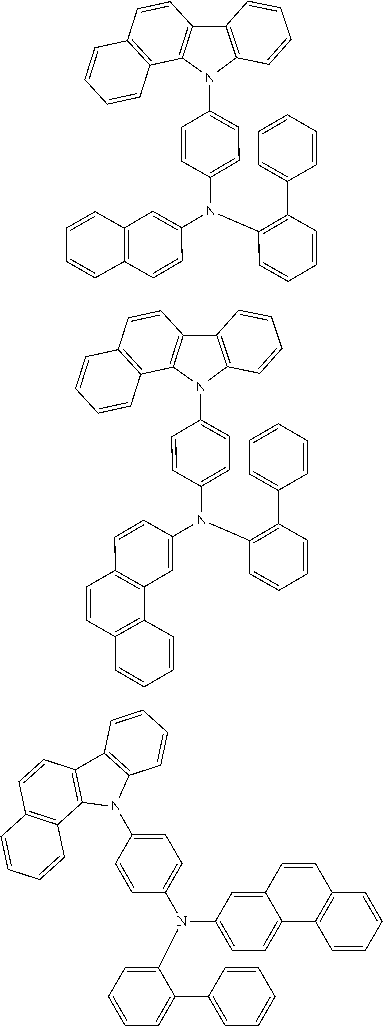 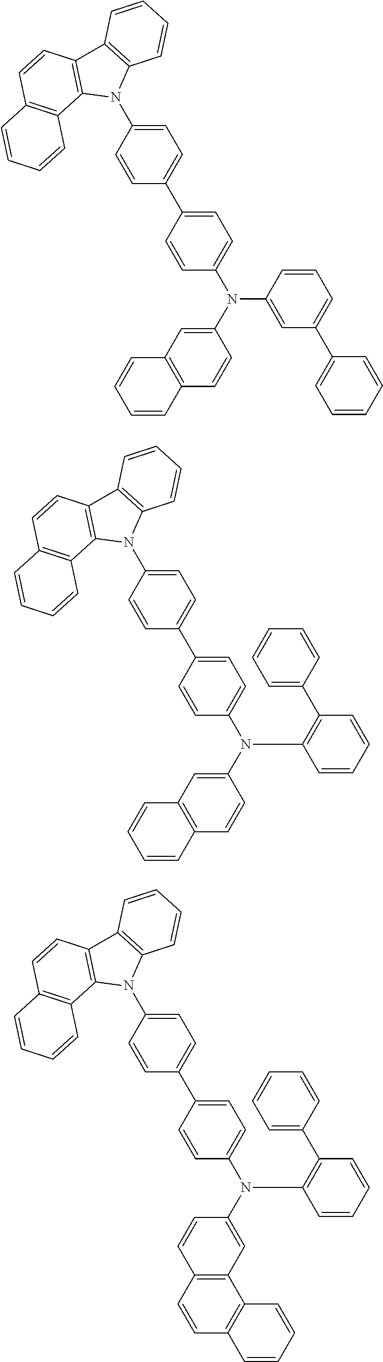 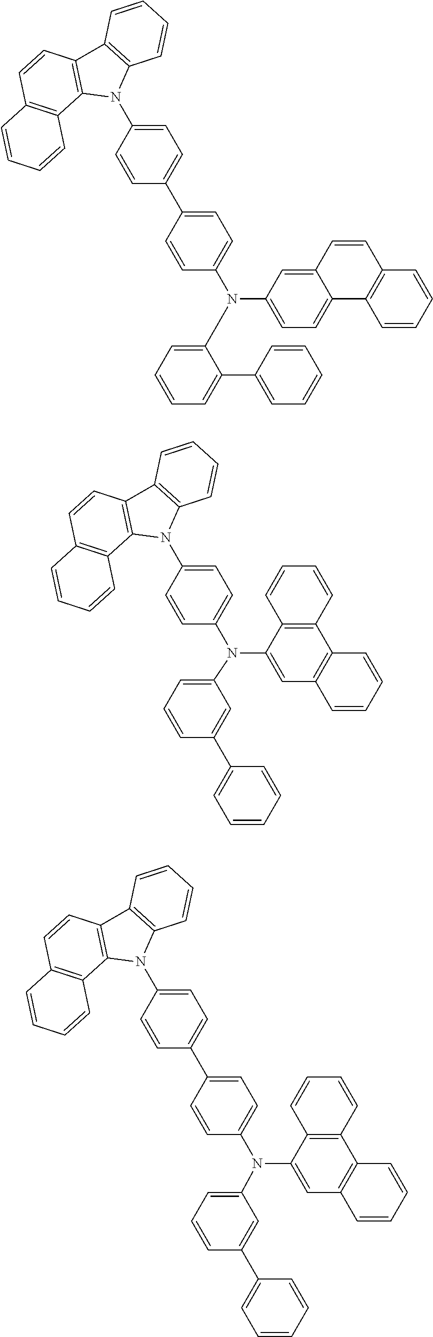 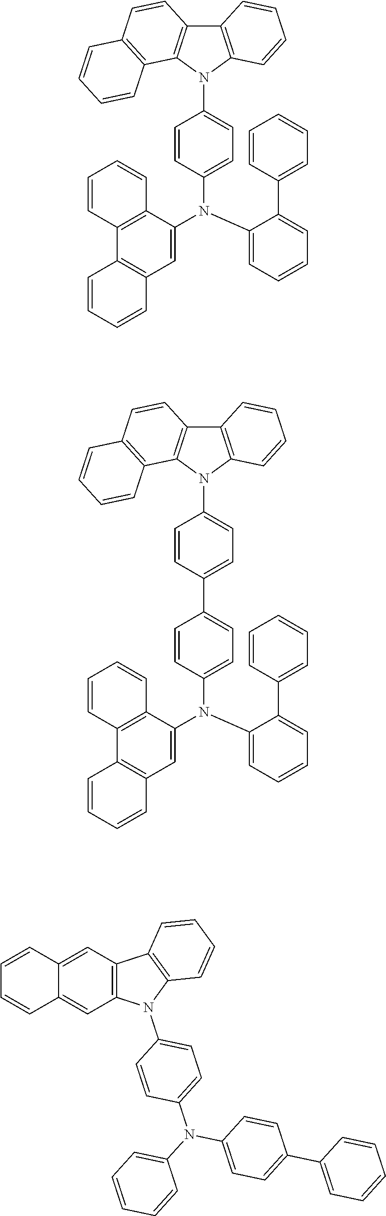 [SMILES: c1ccc(-c2ccccc2N(c2ccc(-n3c4ccccc4c4ccc5ccccc5c43)cc2)c2cc3ccccc3c3ccccc23)cc1 ; c1ccc(-c2ccccc2N(c2ccc(-c3ccc(-n4c5ccccc5c5ccc6ccccc6c54)cc3)cc2)c2cc3ccccc3c3ccccc23)cc1 ; c1ccc(-c2ccc(N(c3ccccc3)c3ccc(-n4c5ccccc5c5cc6ccccc6cc54)cc3)cc2)cc1] 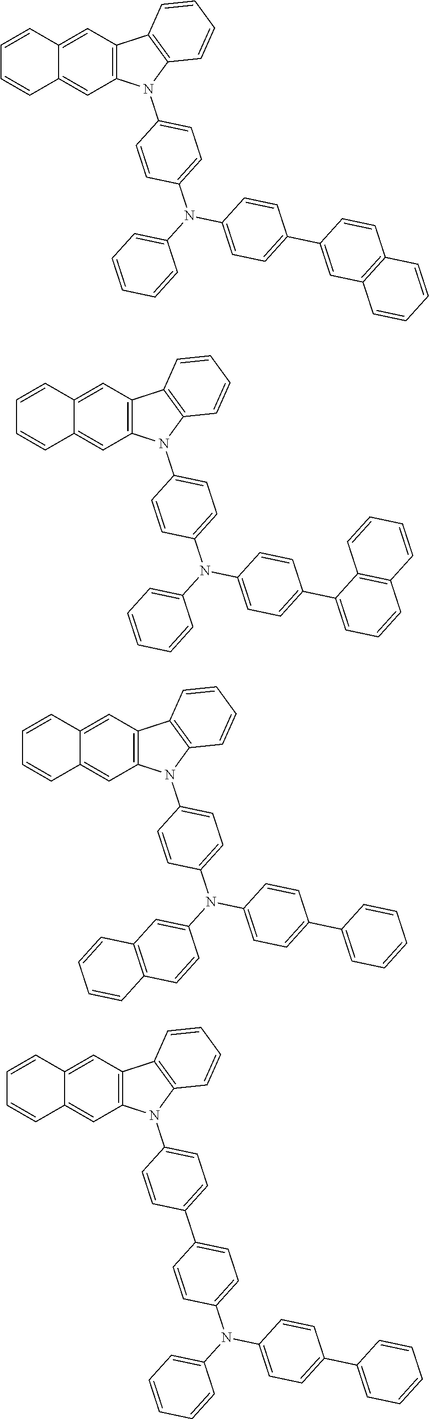 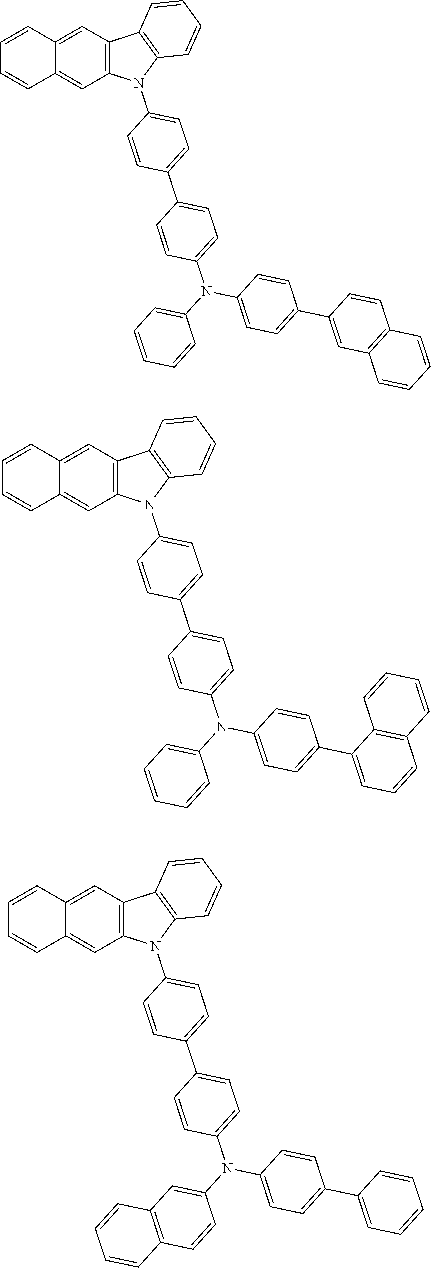 [SMILES: c1ccc(N(c2ccc(-c3ccc(-n4c5ccccc5c5cc6ccccc6cc54)cc3)cc2)c2ccc(-c3ccc4ccccc4c3)cc2)cc1 ; c1ccc(N(c2ccc(-c3ccc(-n4c5ccccc5c5cc6ccccc6cc54)cc3)cc2)c2ccc(-c3cccc4ccccc34)cc2)cc1 ; c1ccc(-c2ccc(N(c3ccc(-c4ccc(-n5c6ccccc6c6cc7ccccc7cc65)cc4)cc3)c3ccc4ccccc4c3)cc2)cc1] 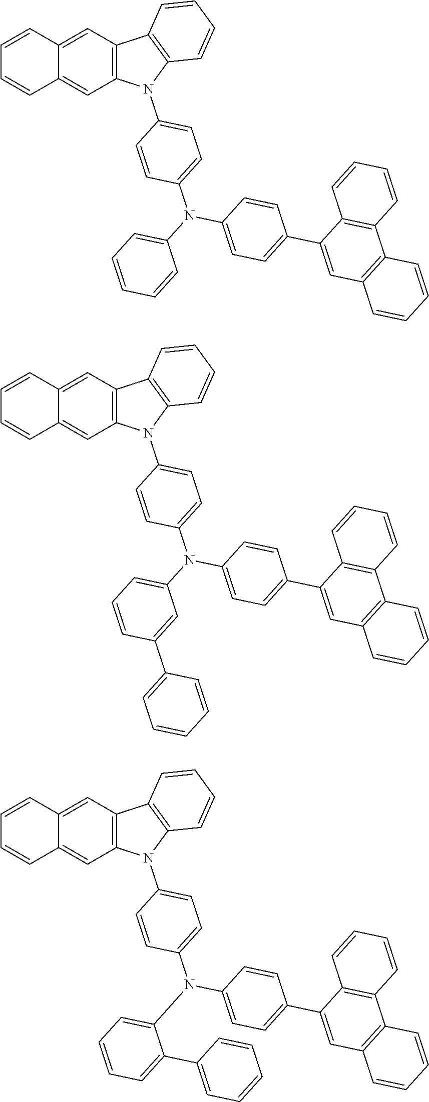 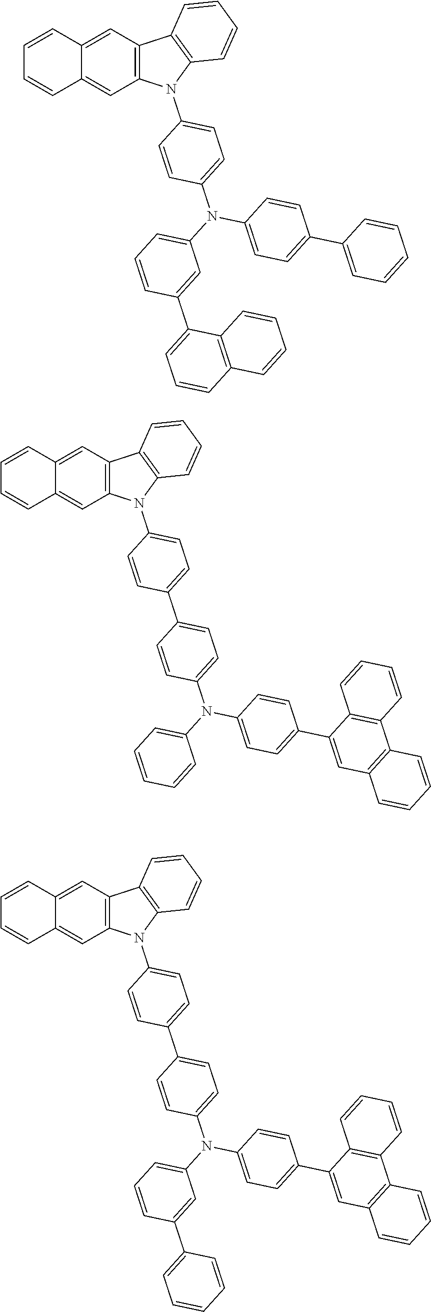 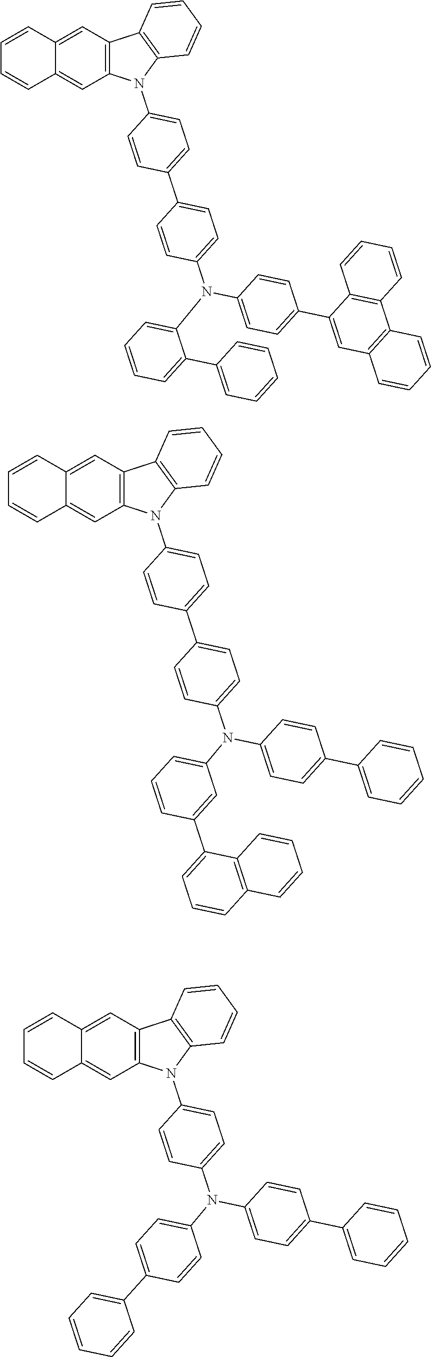 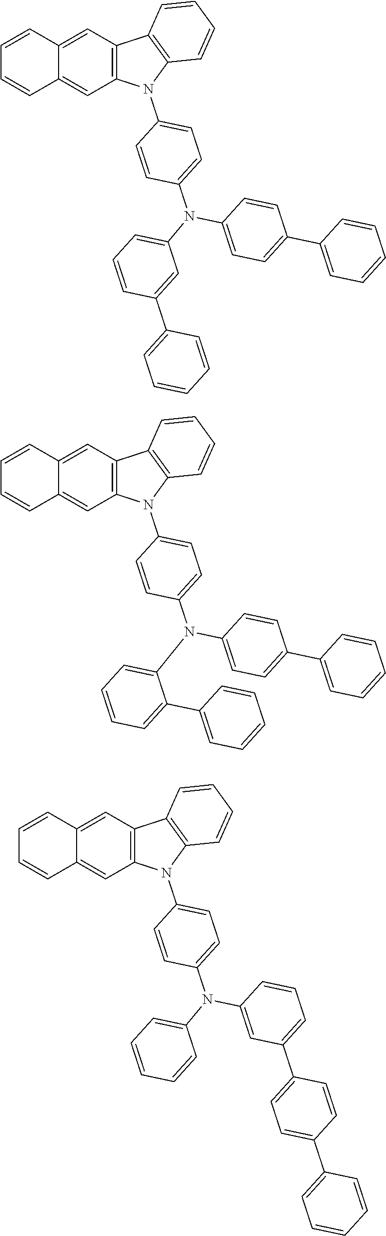 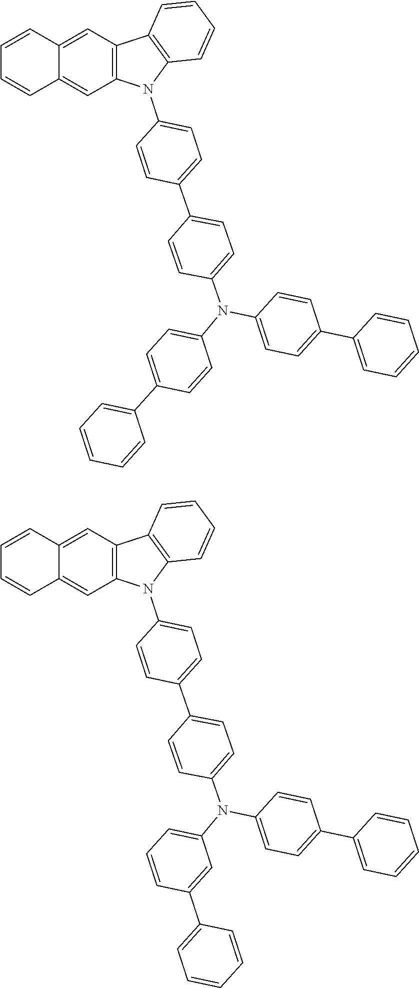 [SMILES: c1ccc(-c2ccc(N(c3ccc(-c4ccccc4)cc3)c3ccc(-c4ccc(-n5c6ccccc6c6cc7ccccc7cc65)cc4)cc3)cc2)cc1 ; c1ccc(-c2ccc(N(c3ccc(-c4ccc(-n5c6ccccc6c6cc7ccccc7cc65)cc4)cc3)c3cccc(-c4ccccc4)c3)cc2)cc1] 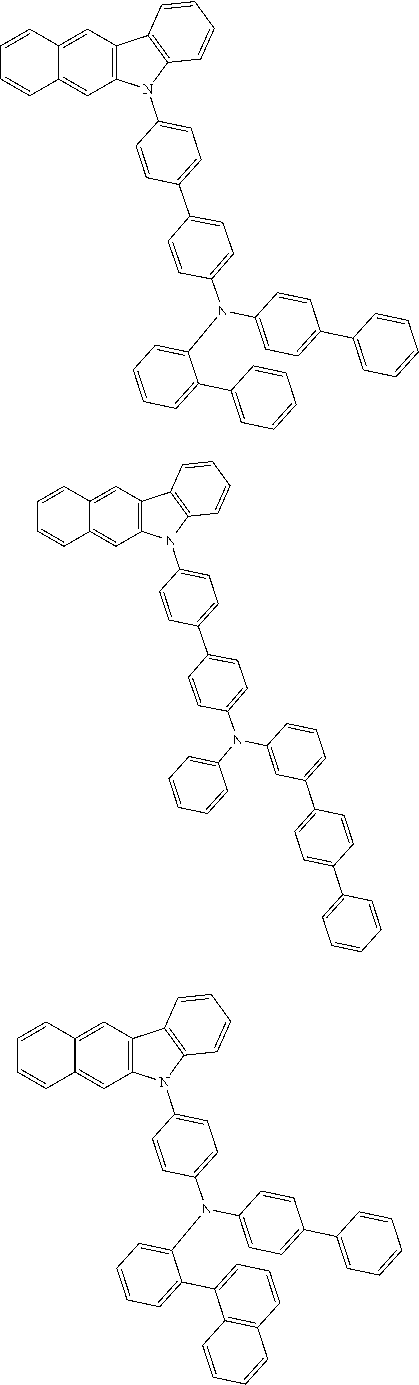 [SMILES: c1ccc(-c2ccc(N(c3ccc(-c4ccc(-n5c6ccccc6c6cc7ccccc7cc65)cc4)cc3)c3ccccc3-c3ccccc3)cc2)cc1 ; c1ccc(-c2ccc(-c3cccc(N(c4ccccc4)c4ccc(-c5ccc(-n6c7ccccc7c7cc8ccccc8cc76)cc5)cc4)c3)cc2)cc1 ; c1ccc(-c2ccc(N(c3ccc(-n4c5ccccc5c5cc6ccccc6cc54)cc3)c3ccccc3-c3cccc4ccccc34)cc2)cc1] 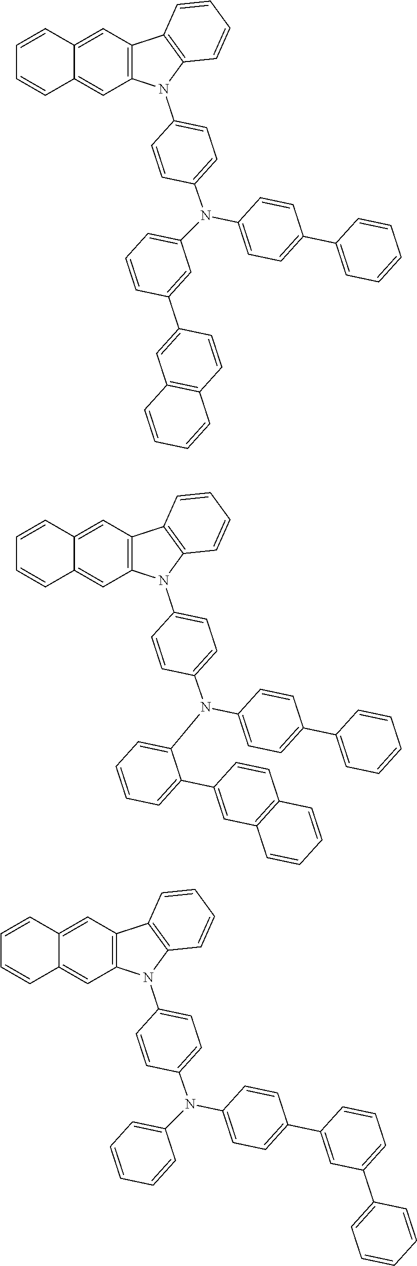 [SMILES: c1ccc(-c2ccc(N(c3ccc(-n4c5ccccc5c5cc6ccccc6cc54)cc3)c3cccc(-c4ccc5ccccc5c4)c3)cc2)cc1 ; c1ccc(-c2ccc(N(c3ccc(-n4c5ccccc5c5cc6ccccc6cc54)cc3)c3ccccc3-c3ccc4ccccc4c3)cc2)cc1 ; c1ccc(-c2cccc(-c3ccc(N(c4ccccc4)c4ccc(-n5c6ccccc6c6cc7ccccc7cc65)cc4)cc3)c2)cc1] 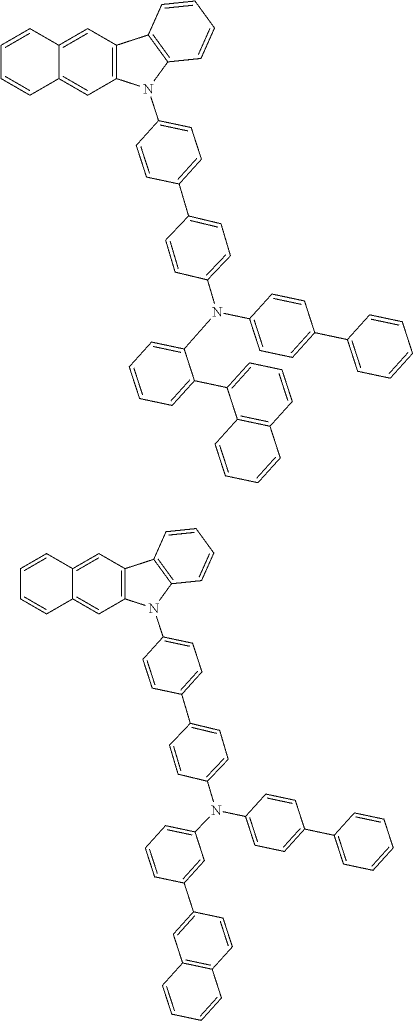 [SMILES: c1ccc(-c2ccc(N(c3ccc(-c4ccc(-n5c6ccccc6c6cc7ccccc7cc65)cc4)cc3)c3ccccc3-c3cccc4ccccc34)cc2)cc1 ; c1ccc(-c2ccc(N(c3ccc(-c4ccc(-n5c6ccccc6c6cc7ccccc7cc65)cc4)cc3)c3cccc(-c4ccc5ccccc5c4)c3)cc2)cc1] 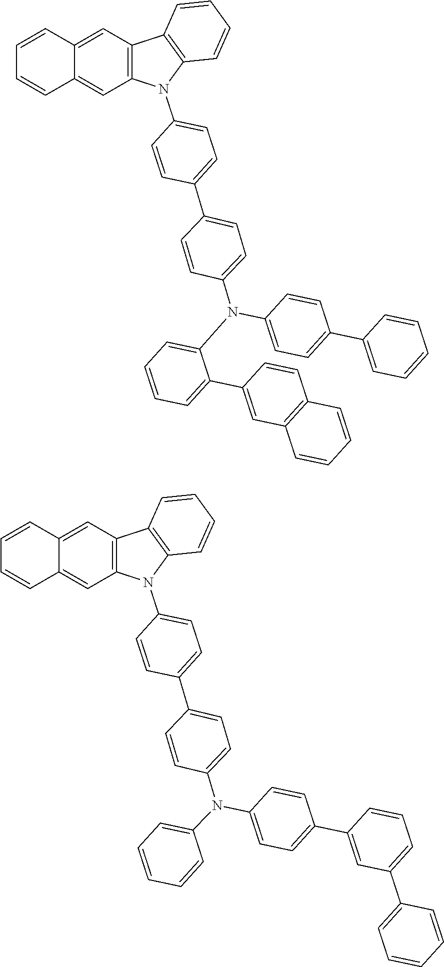 [SMILES: c1ccc(-c2ccc(N(c3ccc(-c4ccc(-n5c6ccccc6c6cc7ccccc7cc65)cc4)cc3)c3ccccc3-c3ccc4ccccc4c3)cc2)cc1 ; c1ccc(-c2cccc(-c3ccc(N(c4ccccc4)c4ccc(-c5ccc(-n6c7ccccc7c7cc8ccccc8cc76)cc5)cc4)cc3)c2)cc1] 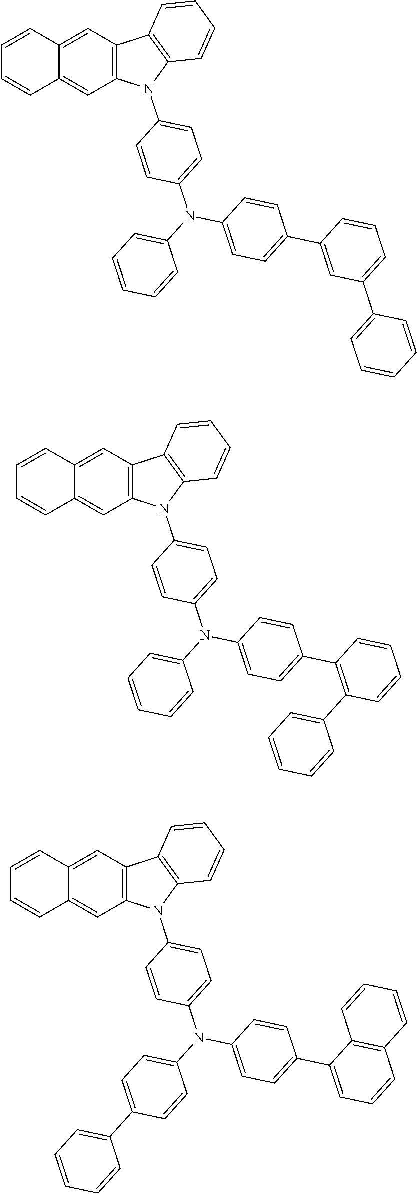 [SMILES: c1ccc(-c2cccc(-c3ccc(N(c4ccccc4)c4ccc(-n5c6ccccc6c6cc7ccccc7cc65)cc4)cc3)c2)cc1 ; c1ccc(-c2ccccc2-c2ccc(N(c3ccccc3)c3ccc(-n4c5ccccc5c5cc6ccccc6cc54)cc3)cc2)cc1 ; c1ccc(-c2ccc(N(c3ccc(-c4cccc5ccccc45)cc3)c3ccc(-n4c5ccccc5c5cc6ccccc6cc54)cc3)cc2)cc1] 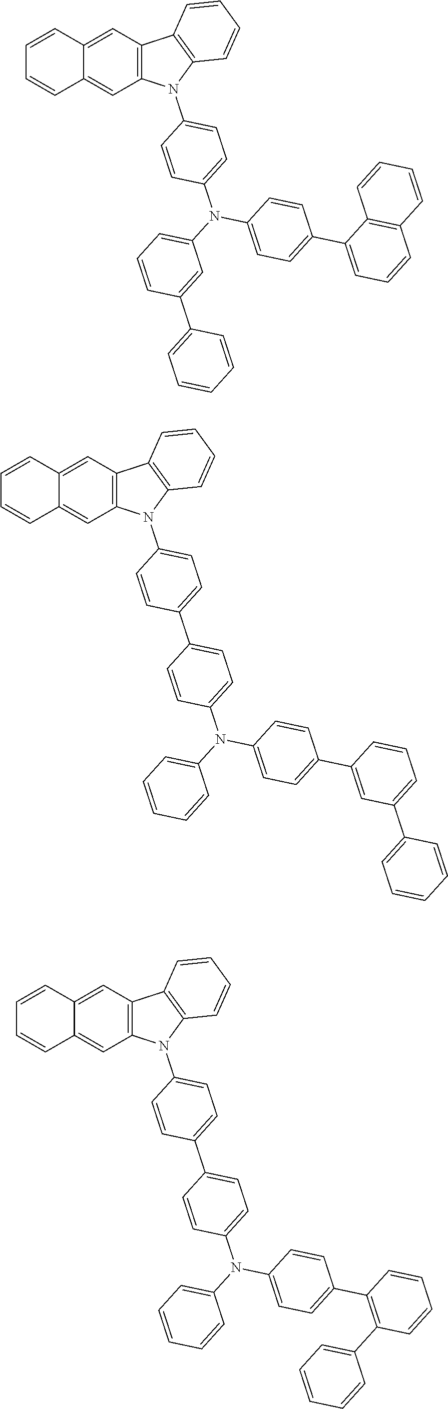 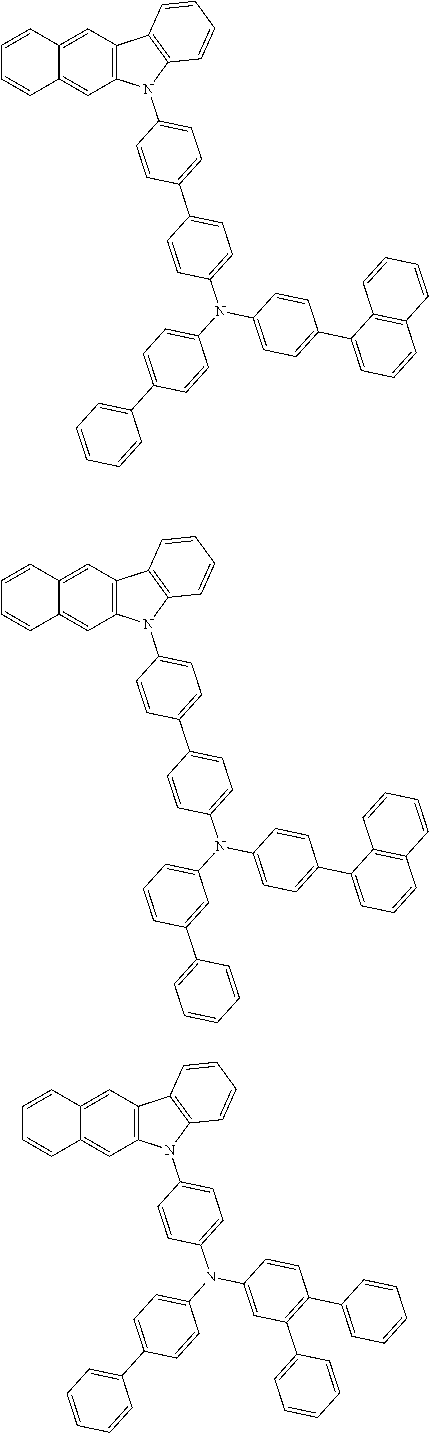 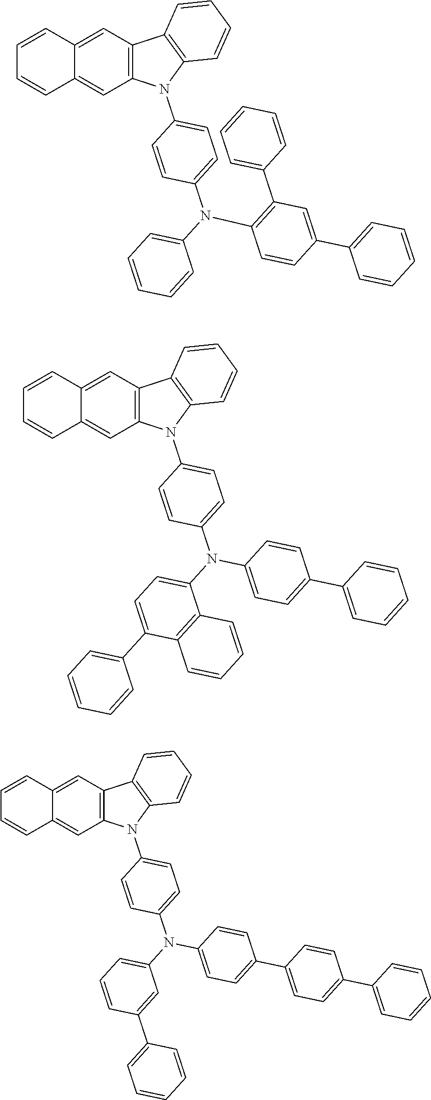 [SMILES: c1ccc(-c2ccc(N(c3ccccc3)c3ccc(-n4c5ccccc5c5cc6ccccc6cc54)cc3)c(-c3ccccc3)c2)cc1 ; c1ccc(-c2ccc(N(c3ccc(-n4c5ccccc5c5cc6ccccc6cc54)cc3)c3ccc(-c4ccccc4)c4ccccc34)cc2)cc1 ; c1ccc(-c2ccc(-c3ccc(N(c4ccc(-n5c6ccccc6c6cc7ccccc7cc65)cc4)c4cccc(-c5ccccc5)c4)cc3)cc2)cc1] 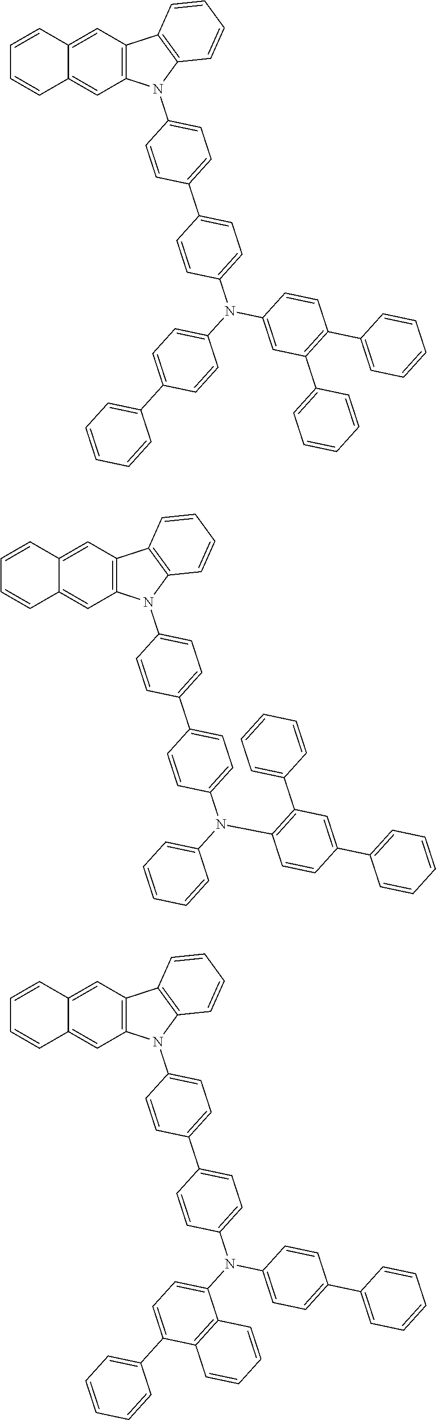 [SMILES: c1ccc(-c2ccc(N(c3ccc(-c4ccc(-n5c6ccccc6c6cc7ccccc7cc65)cc4)cc3)c3ccc(-c4ccccc4)c(-c4ccccc4)c3)cc2)cc1 ; c1ccc(-c2ccc(N(c3ccccc3)c3ccc(-c4ccc(-n5c6ccccc6c6cc7ccccc7cc65)cc4)cc3)c(-c3ccccc3)c2)cc1 ; c1ccc(-c2ccc(N(c3ccc(-c4ccc(-n5c6ccccc6c6cc7ccccc7cc65)cc4)cc3)c3ccc(-c4ccccc4)c4ccccc34)cc2)cc1] 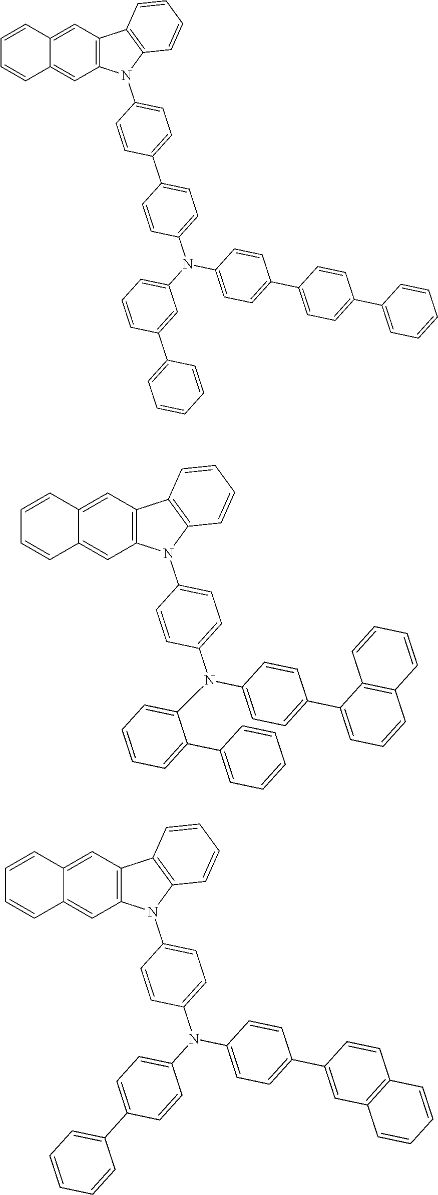 [SMILES: c1ccc(-c2ccc(-c3ccc(N(c4ccc(-c5ccc(-n6c7ccccc7c7cc8ccccc8cc76)cc5)cc4)c4cccc(-c5ccccc5)c4)cc3)cc2)cc1 ; c1ccc(-c2ccccc2N(c2ccc(-c3cccc4ccccc34)cc2)c2ccc(-n3c4ccccc4c4cc5ccccc5cc43)cc2)cc1 ; c1ccc(-c2ccc(N(c3ccc(-c4ccc5ccccc5c4)cc3)c3ccc(-n4c5ccccc5c5cc6ccccc6cc54)cc3)cc2)cc1] 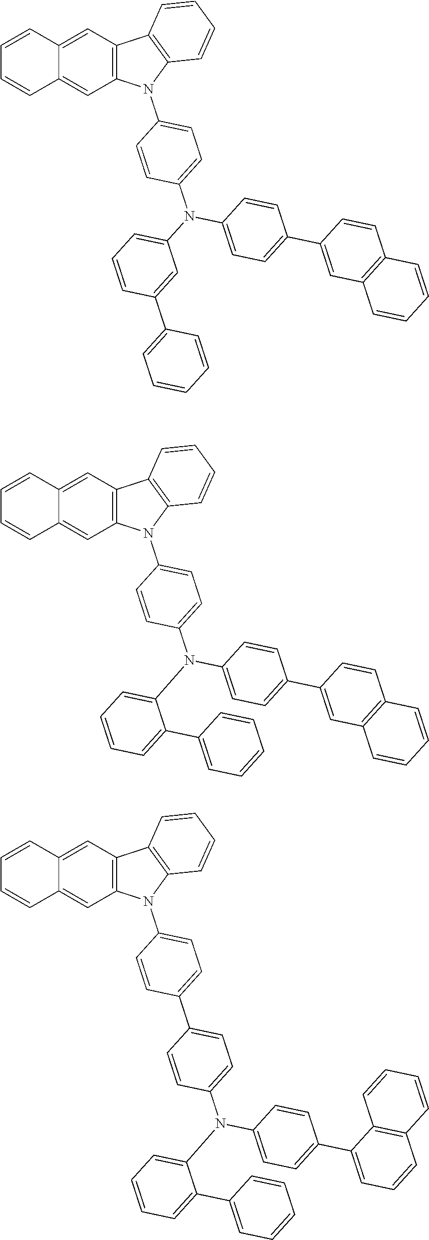 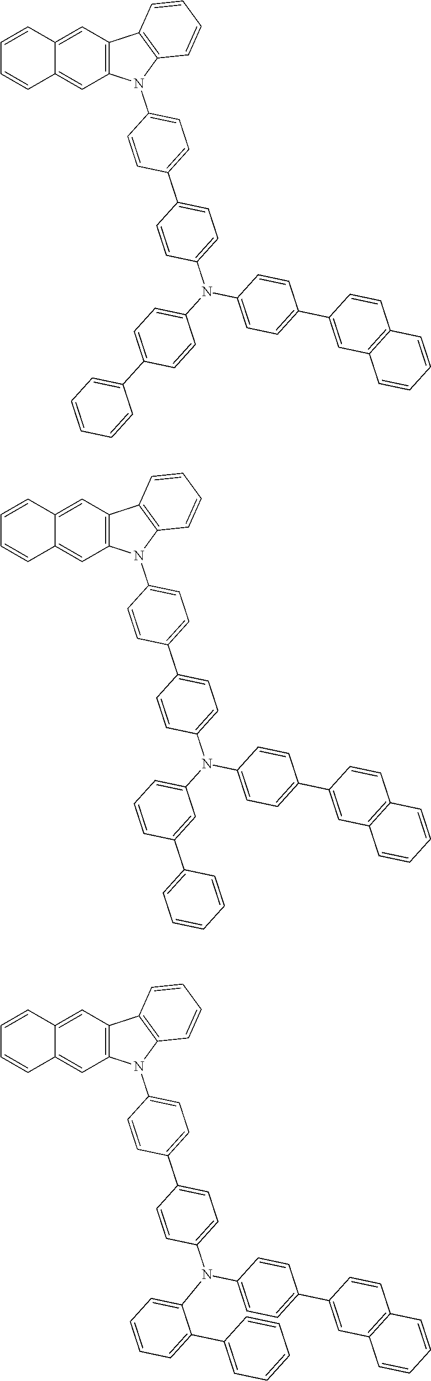 [SMILES: c1ccc(-c2ccc(N(c3ccc(-c4ccc(-n5c6ccccc6c6cc7ccccc7cc65)cc4)cc3)c3ccc(-c4ccc5ccccc5c4)cc3)cc2)cc1 ; c1ccc(-c2cccc(N(c3ccc(-c4ccc(-n5c6ccccc6c6cc7ccccc7cc65)cc4)cc3)c3ccc(-c4ccc5ccccc5c4)cc3)c2)cc1 ; c1ccc(-c2ccccc2N(c2ccc(-c3ccc(-n4c5ccccc5c5cc6ccccc6cc54)cc3)cc2)c2ccc(-c3ccc4ccccc4c3)cc2)cc1] 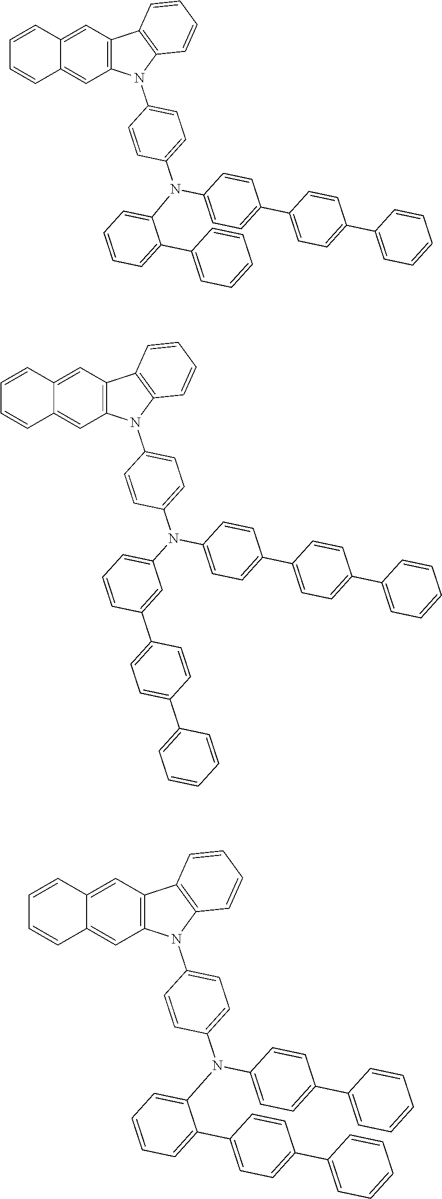 [SMILES: c1ccc(-c2ccc(-c3ccc(N(c4ccc(-n5c6ccccc6c6cc7ccccc7cc65)cc4)c4ccccc4-c4ccccc4)cc3)cc2)cc1 ; c1ccc(-c2ccc(-c3ccc(N(c4ccc(-n5c6ccccc6c6cc7ccccc7cc65)cc4)c4cccc(-c5ccc(-c6ccccc6)cc5)c4)cc3)cc2)cc1 ; c1ccc(-c2ccc(-c3ccccc3N(c3ccc(-c4ccccc4)cc3)c3ccc(-n4c5ccccc5c5cc6ccccc6cc54)cc3)cc2)cc1] 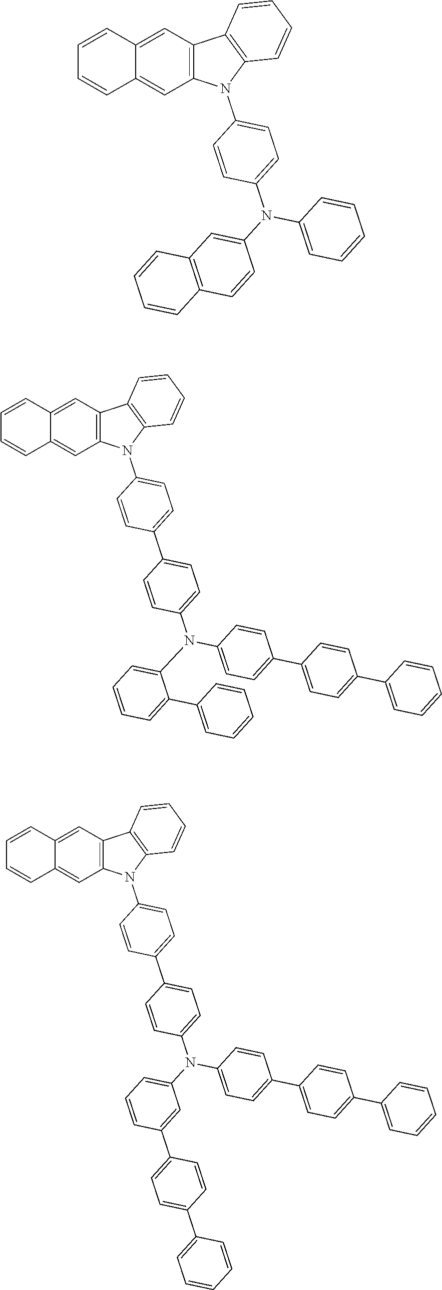 [SMILES: c1ccc(N(c2ccc(-n3c4ccccc4c4cc5ccccc5cc43)cc2)c2ccc3ccccc3c2)cc1 ; c1ccc(-c2ccc(-c3ccc(N(c4ccc(-c5ccc(-n6c7ccccc7c7cc8ccccc8cc76)cc5)cc4)c4ccccc4-c4ccccc4)cc3)cc2)cc1 ; c1ccc(-c2ccc(-c3ccc(N(c4ccc(-c5ccc(-n6c7ccccc7c7cc8ccccc8cc76)cc5)cc4)c4cccc(-c5ccc(-c6ccccc6)cc5)c4)cc3)cc2)cc1] 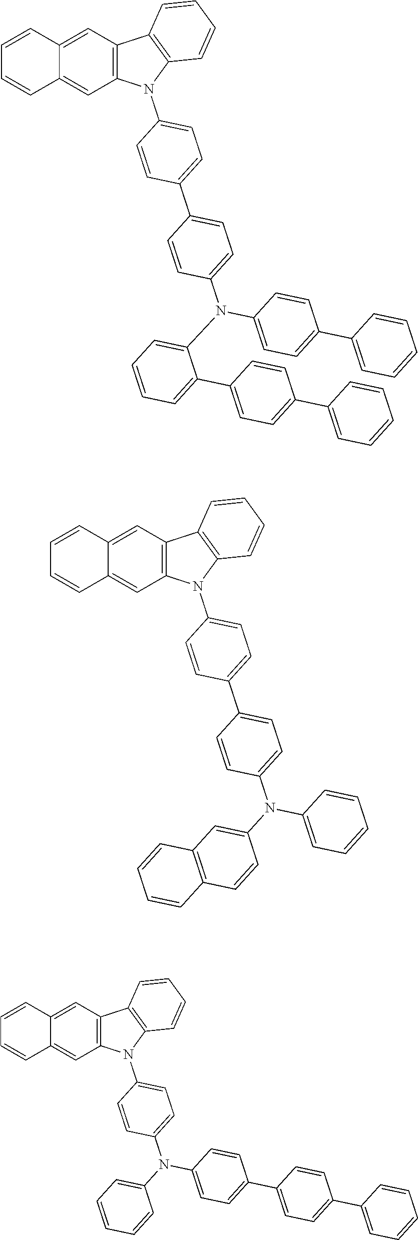 [SMILES: c1ccc(-c2ccc(-c3ccccc3N(c3ccc(-c4ccccc4)cc3)c3ccc(-c4ccc(-n5c6ccccc6c6cc7ccccc7cc65)cc4)cc3)cc2)cc1 ; c1ccc(N(c2ccc(-c3ccc(-n4c5ccccc5c5cc6ccccc6cc54)cc3)cc2)c2ccc3ccccc3c2)cc1 ; c1ccc(-c2ccc(-c3ccc(N(c4ccccc4)c4ccc(-n5c6ccccc6c6cc7ccccc7cc65)cc4)cc3)cc2)cc1] 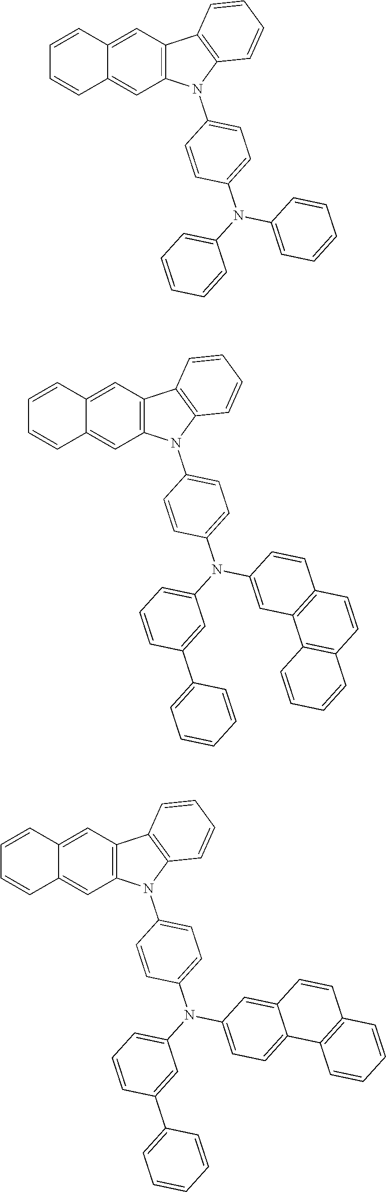 [SMILES: c1ccc(N(c2ccccc2)c2ccc(-n3c4ccccc4c4cc5ccccc5cc43)cc2)cc1 ; c1ccc(-c2cccc(N(c3ccc(-n4c5ccccc5c5cc6ccccc6cc54)cc3)c3ccc4ccc5ccccc5c4c3)c2)cc1 ; c1ccc(-c2cccc(N(c3ccc(-n4c5ccccc5c5cc6ccccc6cc54)cc3)c3ccc4c(ccc5ccccc54)c3)c2)cc1] 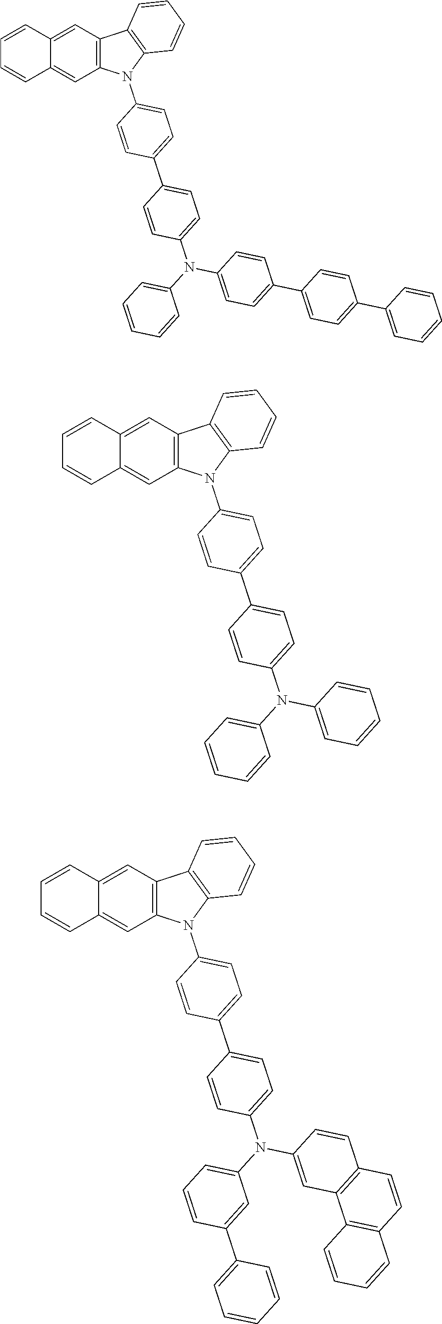 [SMILES: c1ccc(-c2ccc(-c3ccc(N(c4ccccc4)c4ccc(-c5ccc(-n6c7ccccc7c7cc8ccccc8cc76)cc5)cc4)cc3)cc2)cc1 ; c1ccc(N(c2ccccc2)c2ccc(-c3ccc(-n4c5ccccc5c5cc6ccccc6cc54)cc3)cc2)cc1 ; c1ccc(-c2cccc(N(c3ccc(-c4ccc(-n5c6ccccc6c6cc7ccccc7cc65)cc4)cc3)c3ccc4ccc5ccccc5c4c3)c2)cc1] 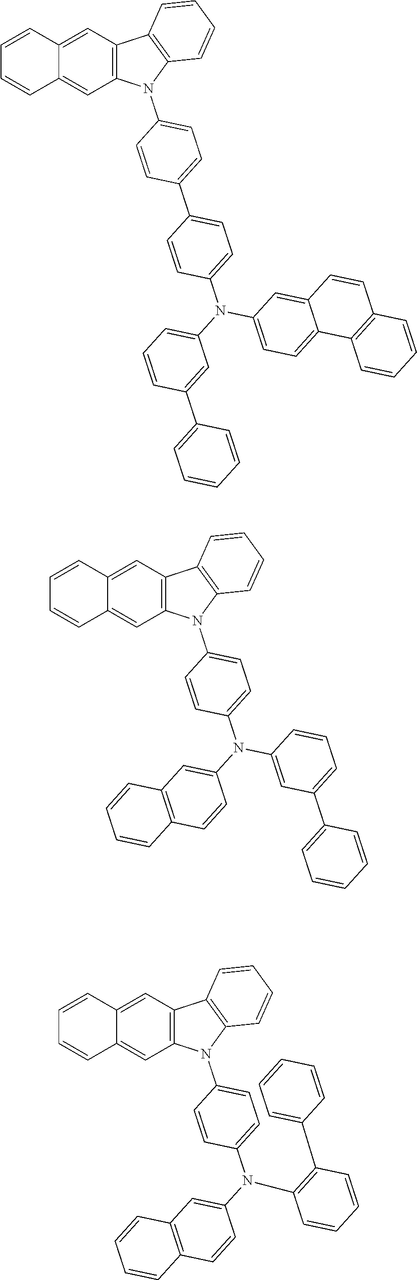 [SMILES: c1ccc(-c2cccc(N(c3ccc(-c4ccc(-n5c6ccccc6c6cc7ccccc7cc65)cc4)cc3)c3ccc4c(ccc5ccccc54)c3)c2)cc1 ; c1ccc(-c2cccc(N(c3ccc(-n4c5ccccc5c5cc6ccccc6cc54)cc3)c3ccc4ccccc4c3)c2)cc1 ; c1ccc(-c2ccccc2N(c2ccc(-n3c4ccccc4c4cc5ccccc5cc43)cc2)c2ccc3ccccc3c2)cc1] 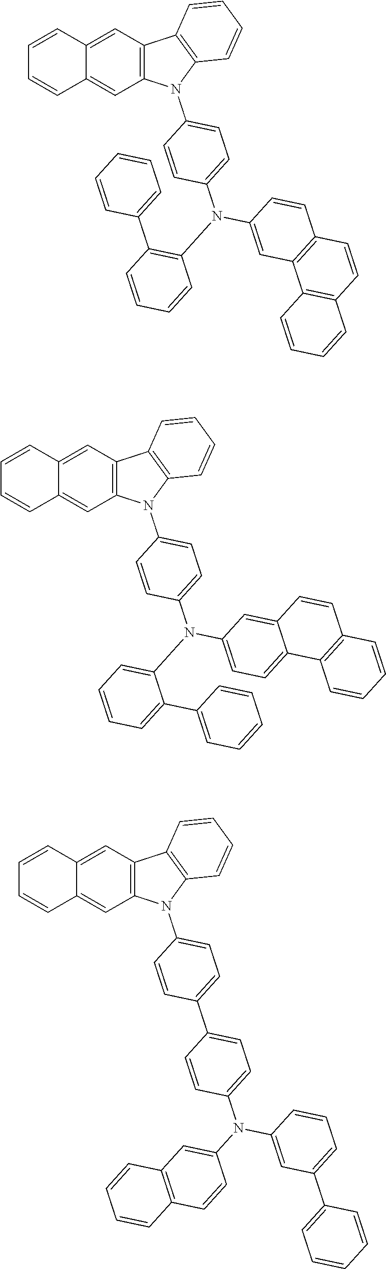 [SMILES: c1ccc(-c2ccccc2N(c2ccc(-n3c4ccccc4c4cc5ccccc5cc43)cc2)c2ccc3ccc4ccccc4c3c2)cc1 ; c1ccc(-c2ccccc2N(c2ccc(-n3c4ccccc4c4cc5ccccc5cc43)cc2)c2ccc3c(ccc4ccccc43)c2)cc1 ; c1ccc(-c2cccc(N(c3ccc(-c4ccc(-n5c6ccccc6c6cc7ccccc7cc65)cc4)cc3)c3ccc4ccccc4c3)c2)cc1] 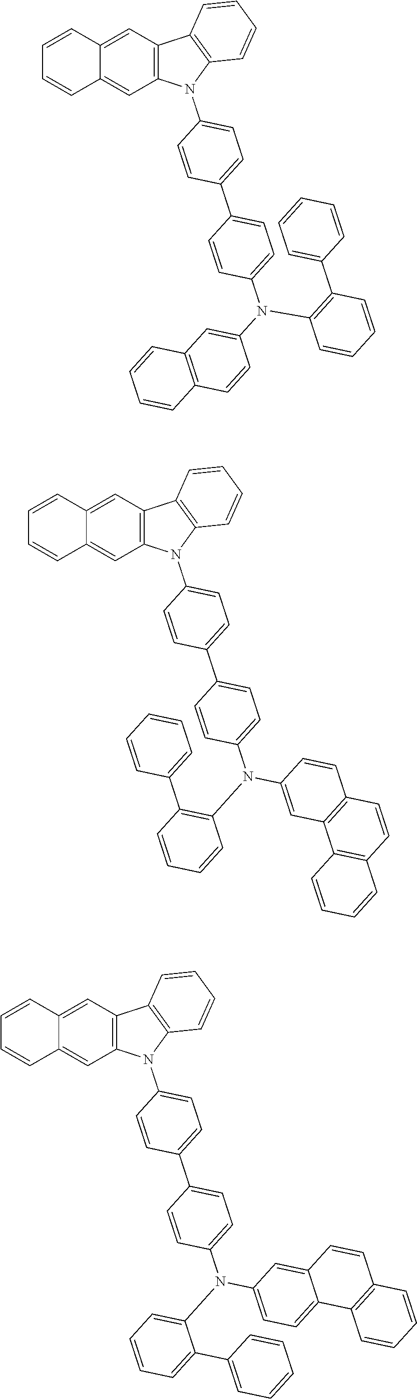 [SMILES: c1ccc(-c2ccccc2N(c2ccc(-c3ccc(-n4c5ccccc5c5cc6ccccc6cc54)cc3)cc2)c2ccc3ccccc3c2)cc1 ; c1ccc(-c2ccccc2N(c2ccc(-c3ccc(-n4c5ccccc5c5cc6ccccc6cc54)cc3)cc2)c2ccc3ccc4ccccc4c3c2)cc1 ; c1ccc(-c2ccccc2N(c2ccc(-c3ccc(-n4c5ccccc5c5cc6ccccc6cc54)cc3)cc2)c2ccc3c(ccc4ccccc43)c2)cc1] 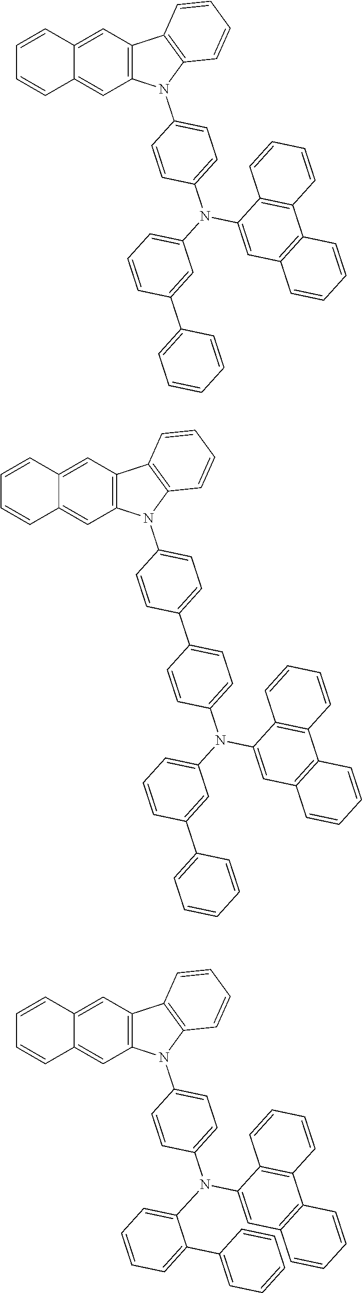 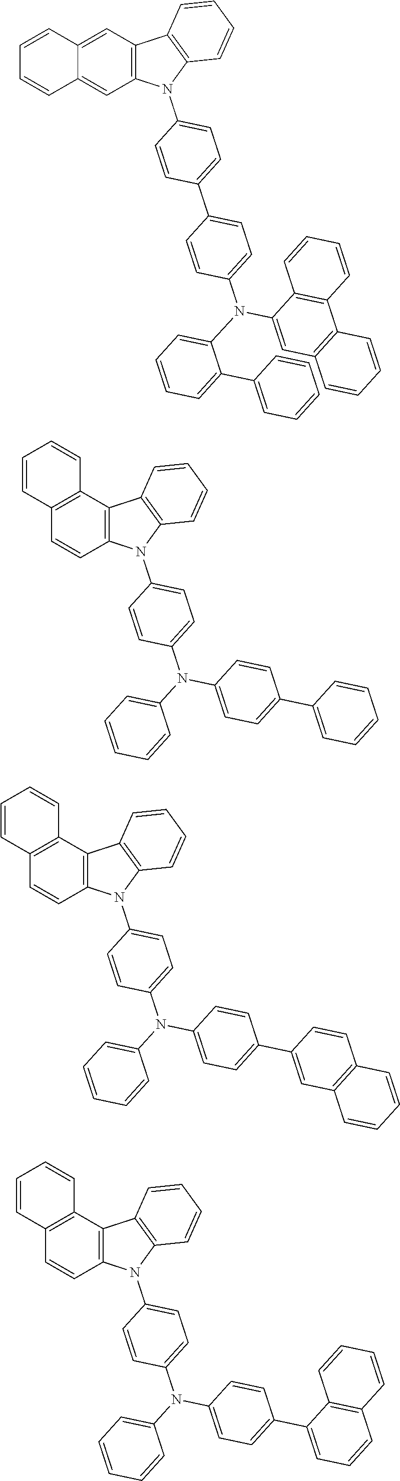 [SMILES: c1ccc(-c2ccccc2N(c2ccc(-c3ccc(-n4c5ccccc5c5cc6ccccc6cc54)cc3)cc2)c2cc3ccccc3c3ccccc23)cc1 ; c1ccc(-c2ccc(N(c3ccccc3)c3ccc(-n4c5ccccc5c5c6ccccc6ccc54)cc3)cc2)cc1 ; c1ccc(N(c2ccc(-c3ccc4ccccc4c3)cc2)c2ccc(-n3c4ccccc4c4c5ccccc5ccc43)cc2)cc1 ; c1ccc(N(c2ccc(-c3cccc4ccccc34)cc2)c2ccc(-n3c4ccccc4c4c5ccccc5ccc43)cc2)cc1] 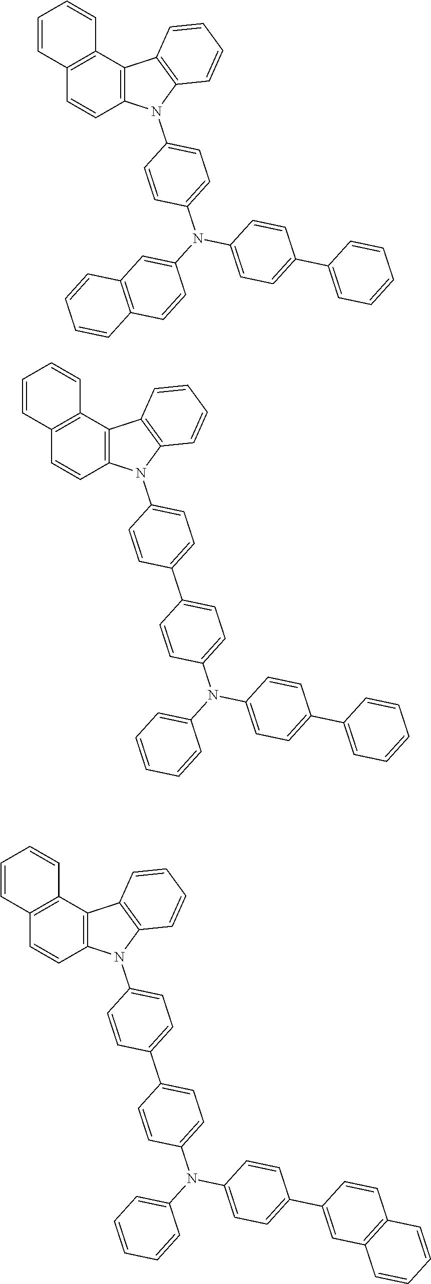 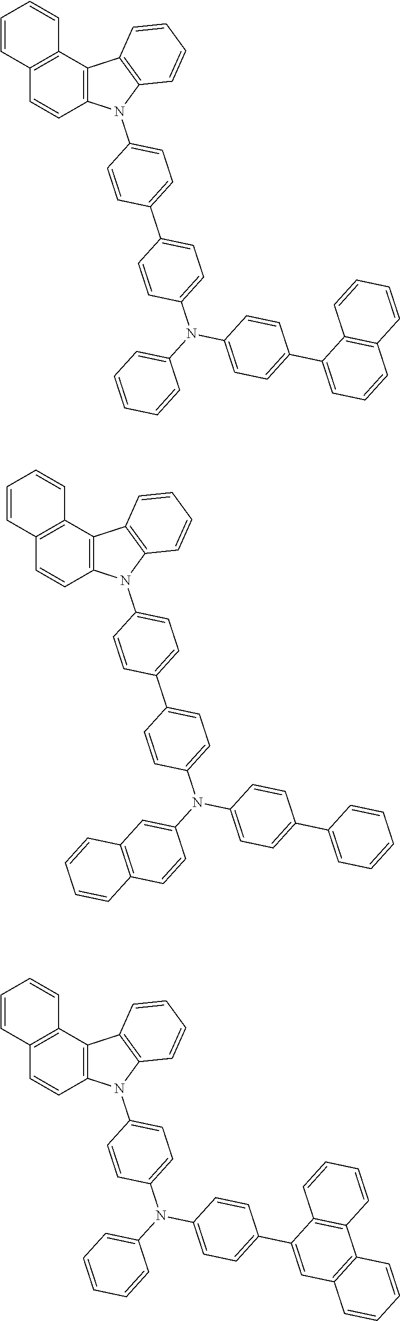 [SMILES: c1ccc(N(c2ccc(-c3ccc(-n4c5ccccc5c5c6ccccc6ccc54)cc3)cc2)c2ccc(-c3cccc4ccccc34)cc2)cc1 ; c1ccc(-c2ccc(N(c3ccc(-c4ccc(-n5c6ccccc6c6c7ccccc7ccc65)cc4)cc3)c3ccc4ccccc4c3)cc2)cc1 ; c1ccc(N(c2ccc(-c3cc4ccccc4c4ccccc34)cc2)c2ccc(-n3c4ccccc4c4c5ccccc5ccc43)cc2)cc1] 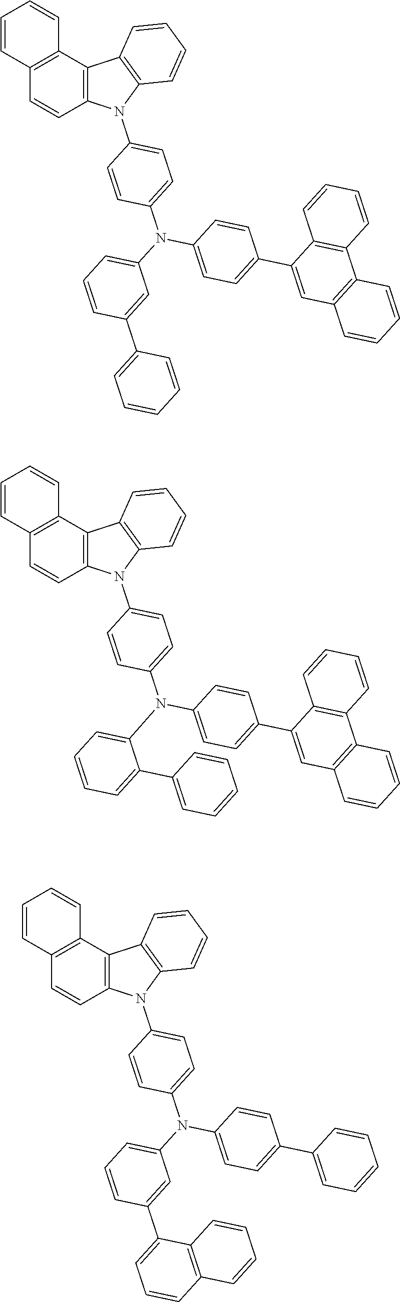 [SMILES: c1ccc(-c2cccc(N(c3ccc(-c4cc5ccccc5c5ccccc45)cc3)c3ccc(-n4c5ccccc5c5c6ccccc6ccc54)cc3)c2)cc1 ; c1ccc(-c2ccccc2N(c2ccc(-c3cc4ccccc4c4ccccc34)cc2)c2ccc(-n3c4ccccc4c4c5ccccc5ccc43)cc2)cc1 ; c1ccc(-c2ccc(N(c3ccc(-n4c5ccccc5c5c6ccccc6ccc54)cc3)c3cccc(-c4cccc5ccccc45)c3)cc2)cc1] 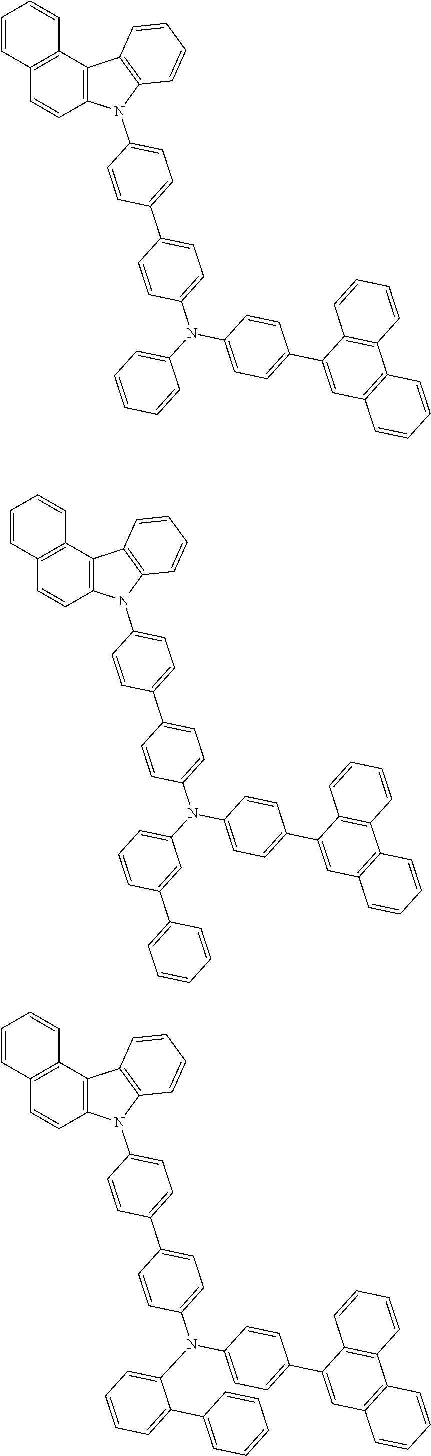 [SMILES: c1ccc(N(c2ccc(-c3ccc(-n4c5ccccc5c5c6ccccc6ccc54)cc3)cc2)c2ccc(-c3cc4ccccc4c4ccccc34)cc2)cc1 ; c1ccc(-c2cccc(N(c3ccc(-c4ccc(-n5c6ccccc6c6c7ccccc7ccc65)cc4)cc3)c3ccc(-c4cc5ccccc5c5ccccc45)cc3)c2)cc1 ; c1ccc(-c2ccccc2N(c2ccc(-c3ccc(-n4c5ccccc5c5c6ccccc6ccc54)cc3)cc2)c2ccc(-c3cc4ccccc4c4ccccc34)cc2)cc1] 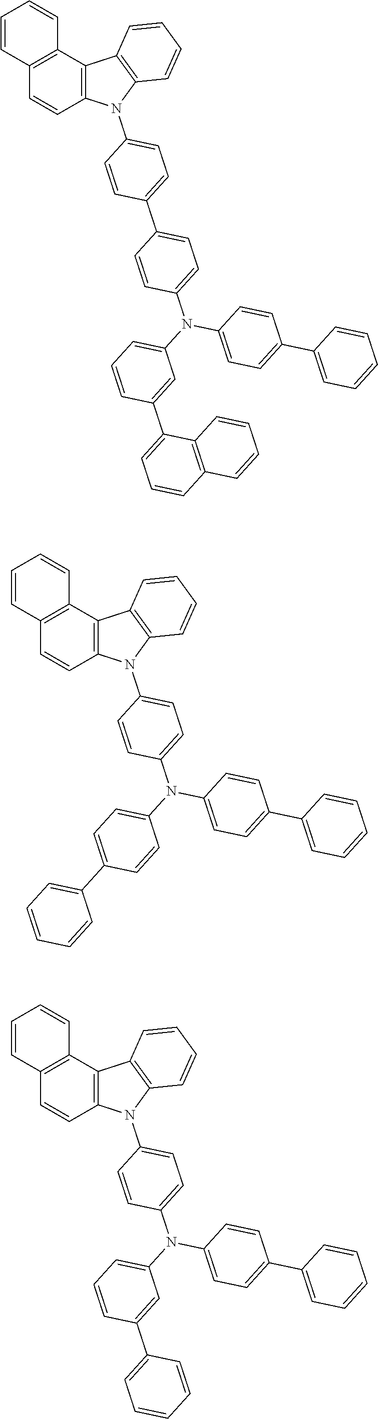 [SMILES: c1ccc(-c2ccc(N(c3ccc(-c4ccc(-n5c6ccccc6c6c7ccccc7ccc65)cc4)cc3)c3cccc(-c4cccc5ccccc45)c3)cc2)cc1 ; c1ccc(-c2ccc(N(c3ccc(-c4ccccc4)cc3)c3ccc(-n4c5ccccc5c5c6ccccc6ccc54)cc3)cc2)cc1 ; c1ccc(-c2ccc(N(c3ccc(-n4c5ccccc5c5c6ccccc6ccc54)cc3)c3cccc(-c4ccccc4)c3)cc2)cc1] 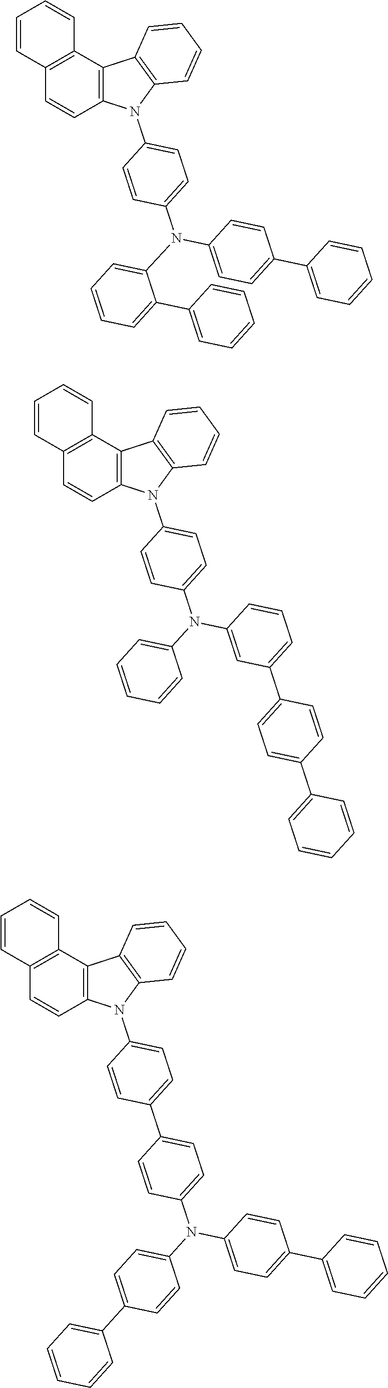 [SMILES: c1ccc(-c2ccc(N(c3ccc(-n4c5ccccc5c5c6ccccc6ccc54)cc3)c3ccccc3-c3ccccc3)cc2)cc1 ; c1ccc(-c2ccc(-c3cccc(N(c4ccccc4)c4ccc(-n5c6ccccc6c6c7ccccc7ccc65)cc4)c3)cc2)cc1 ; c1ccc(-c2ccc(N(c3ccc(-c4ccccc4)cc3)c3ccc(-c4ccc(-n5c6ccccc6c6c7ccccc7ccc65)cc4)cc3)cc2)cc1] 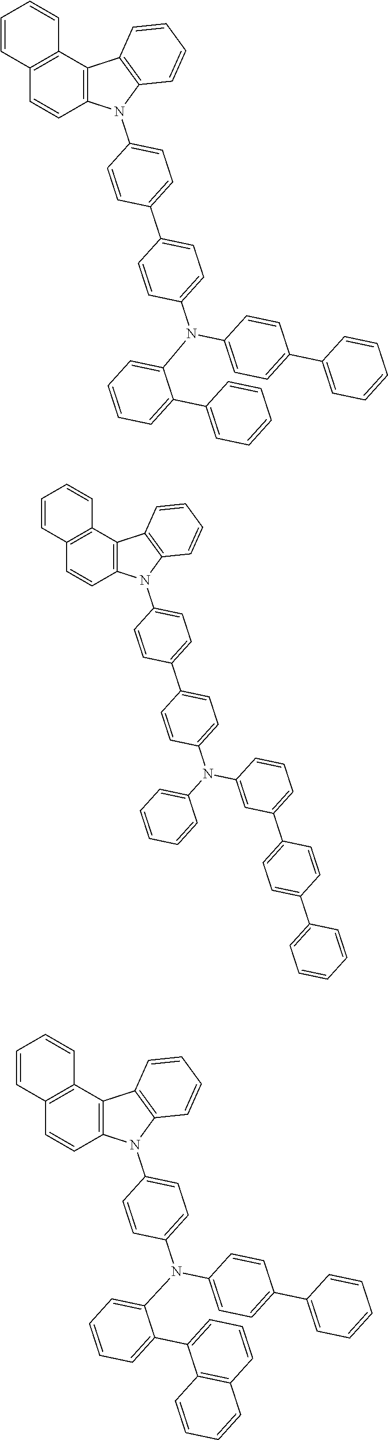 [SMILES: c1ccc(-c2ccc(N(c3ccc(-c4ccc(-n5c6ccccc6c6c7ccccc7ccc65)cc4)cc3)c3ccccc3-c3ccccc3)cc2)cc1 ; c1ccc(-c2ccc(-c3cccc(N(c4ccccc4)c4ccc(-c5ccc(-n6c7ccccc7c7c8ccccc8ccc76)cc5)cc4)c3)cc2)cc1 ; c1ccc(-c2ccc(N(c3ccc(-n4c5ccccc5c5c6ccccc6ccc54)cc3)c3ccccc3-c3cccc4ccccc34)cc2)cc1] 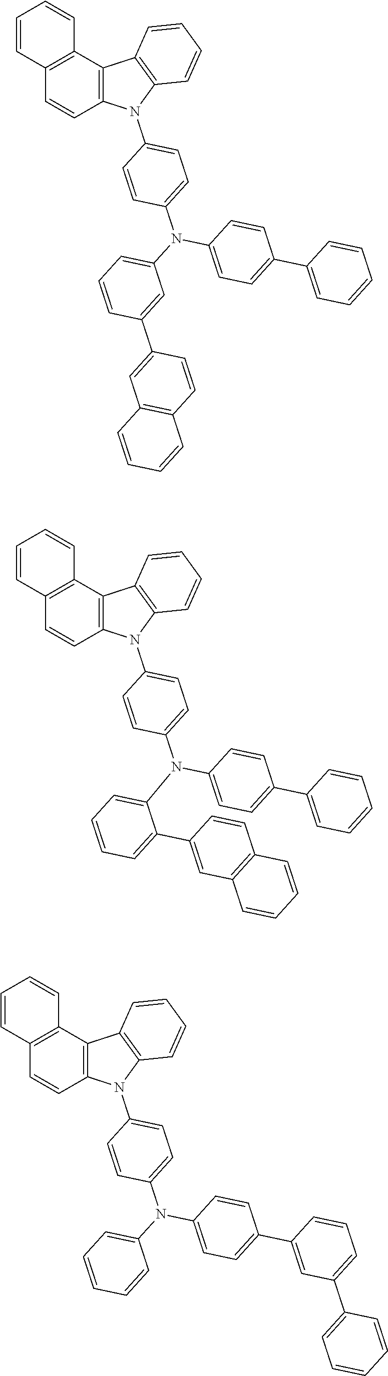 [SMILES: c1ccc(-c2ccc(N(c3ccc(-n4c5ccccc5c5c6ccccc6ccc54)cc3)c3cccc(-c4ccc5ccccc5c4)c3)cc2)cc1 ; c1ccc(-c2ccc(N(c3ccc(-n4c5ccccc5c5c6ccccc6ccc54)cc3)c3ccccc3-c3ccc4ccccc4c3)cc2)cc1 ; c1ccc(-c2cccc(-c3ccc(N(c4ccccc4)c4ccc(-n5c6ccccc6c6c7ccccc7ccc65)cc4)cc3)c2)cc1] 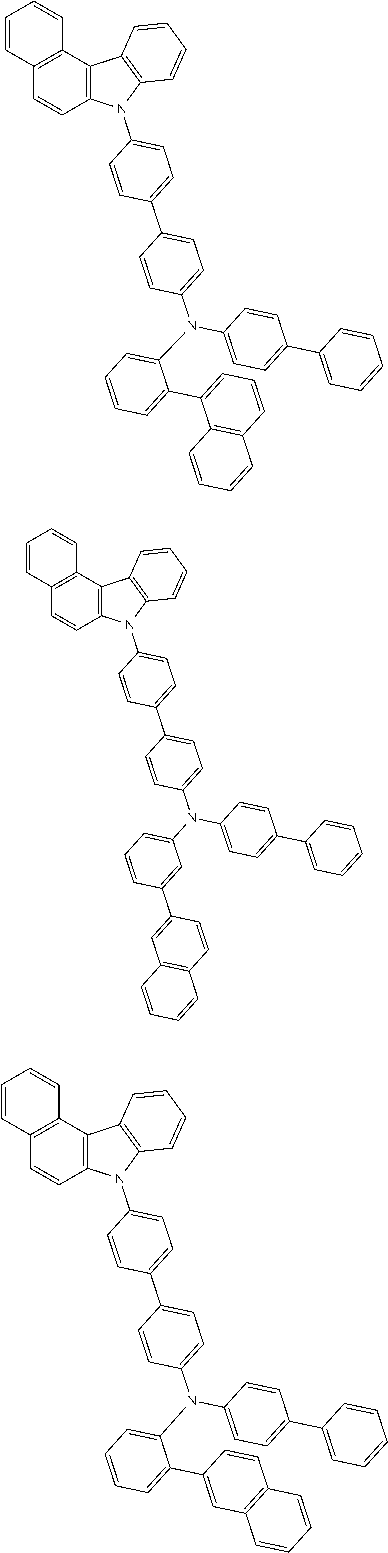 [SMILES: c1ccc(-c2ccc(N(c3ccc(-c4ccc(-n5c6ccccc6c6c7ccccc7ccc65)cc4)cc3)c3ccccc3-c3cccc4ccccc34)cc2)cc1 ; c1ccc(-c2ccc(N(c3ccc(-c4ccc(-n5c6ccccc6c6c7ccccc7ccc65)cc4)cc3)c3cccc(-c4ccc5ccccc5c4)c3)cc2)cc1 ; c1ccc(-c2ccc(N(c3ccc(-c4ccc(-n5c6ccccc6c6c7ccccc7ccc65)cc4)cc3)c3ccccc3-c3ccc4ccccc4c3)cc2)cc1] 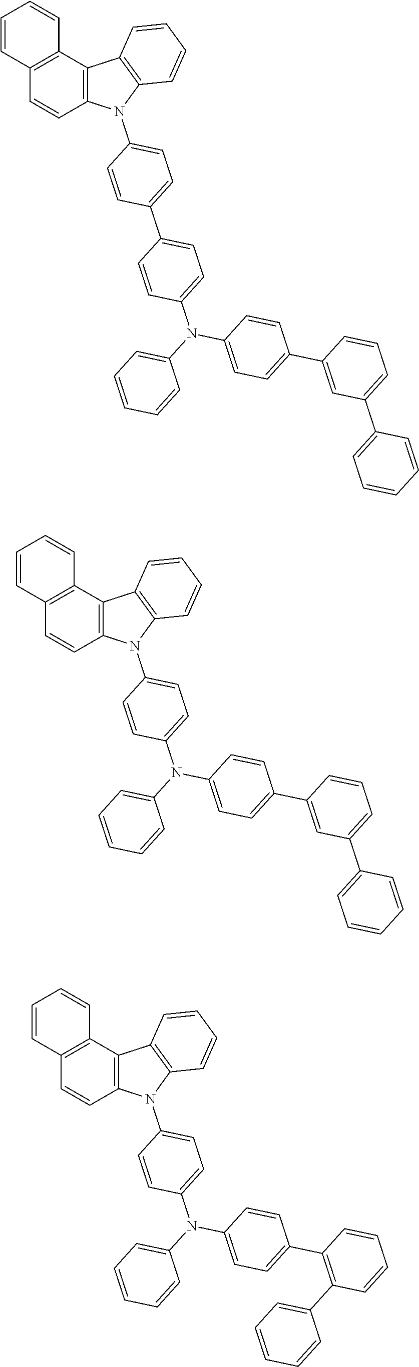 [SMILES: c1ccc(-c2cccc(-c3ccc(N(c4ccccc4)c4ccc(-c5ccc(-n6c7ccccc7c7c8ccccc8ccc76)cc5)cc4)cc3)c2)cc1 ; c1ccc(-c2cccc(-c3ccc(N(c4ccccc4)c4ccc(-n5c6ccccc6c6c7ccccc7ccc65)cc4)cc3)c2)cc1 ; c1ccc(-c2ccccc2-c2ccc(N(c3ccccc3)c3ccc(-n4c5ccccc5c5c6ccccc6ccc54)cc3)cc2)cc1] 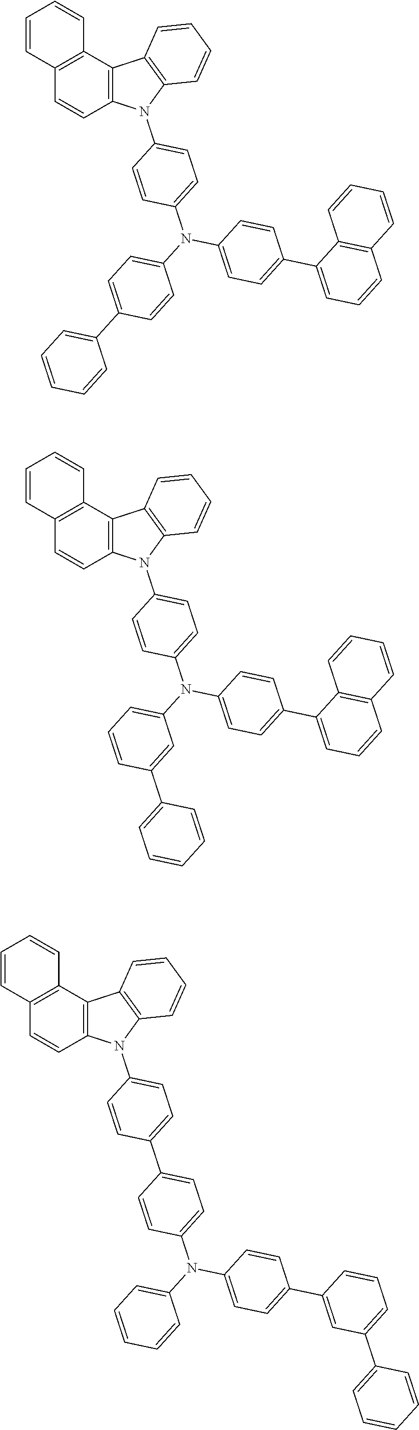 [SMILES: c1ccc(-c2ccc(N(c3ccc(-c4cccc5ccccc45)cc3)c3ccc(-n4c5ccccc5c5c6ccccc6ccc54)cc3)cc2)cc1 ; c1ccc(-c2cccc(N(c3ccc(-c4cccc5ccccc45)cc3)c3ccc(-n4c5ccccc5c5c6ccccc6ccc54)cc3)c2)cc1 ; c1ccc(-c2cccc(-c3ccc(N(c4ccccc4)c4ccc(-c5ccc(-n6c7ccccc7c7c8ccccc8ccc76)cc5)cc4)cc3)c2)cc1] 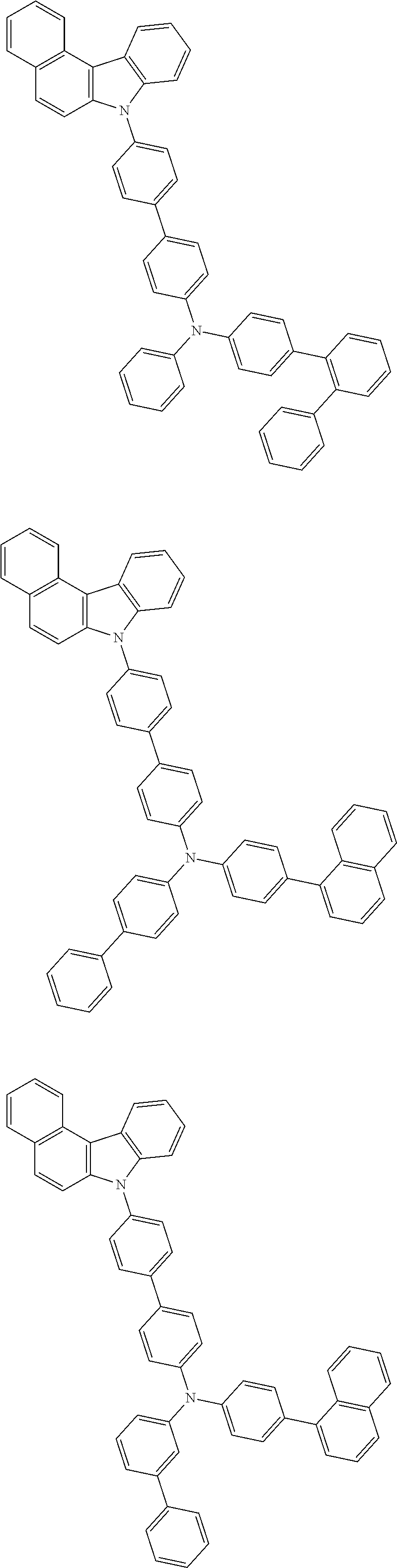 [SMILES: c1ccc(-c2ccccc2-c2ccc(N(c3ccccc3)c3ccc(-c4ccc(-n5c6ccccc6c6c7ccccc7ccc65)cc4)cc3)cc2)cc1 ; c1ccc(-c2ccc(N(c3ccc(-c4ccc(-n5c6ccccc6c6c7ccccc7ccc65)cc4)cc3)c3ccc(-c4cccc5ccccc45)cc3)cc2)cc1 ; c1ccc(-c2cccc(N(c3ccc(-c4ccc(-n5c6ccccc6c6c7ccccc7ccc65)cc4)cc3)c3ccc(-c4cccc5ccccc45)cc3)c2)cc1] 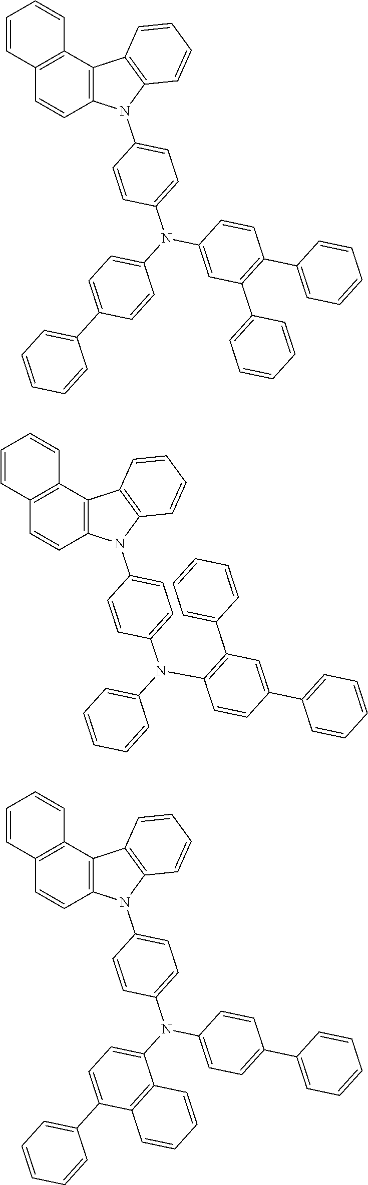 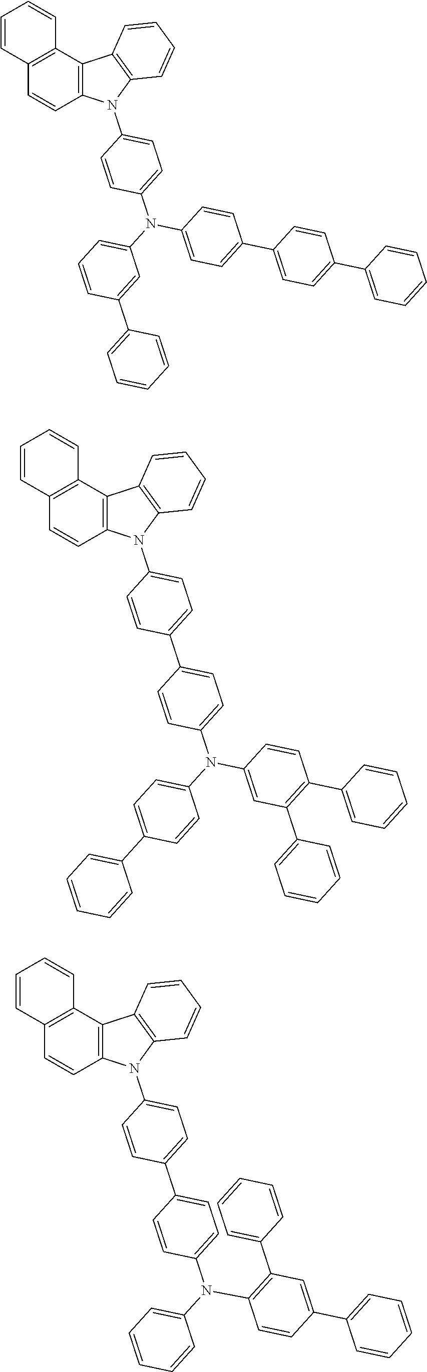 [SMILES: c1ccc(-c2ccc(-c3ccc(N(c4ccc(-n5c6ccccc6c6c7ccccc7ccc65)cc4)c4cccc(-c5ccccc5)c4)cc3)cc2)cc1 ; c1ccc(-c2ccc(N(c3ccc(-c4ccc(-n5c6ccccc6c6c7ccccc7ccc65)cc4)cc3)c3ccc(-c4ccccc4)c(-c4ccccc4)c3)cc2)cc1 ; c1ccc(-c2ccc(N(c3ccccc3)c3ccc(-c4ccc(-n5c6ccccc6c6c7ccccc7ccc65)cc4)cc3)c(-c3ccccc3)c2)cc1] 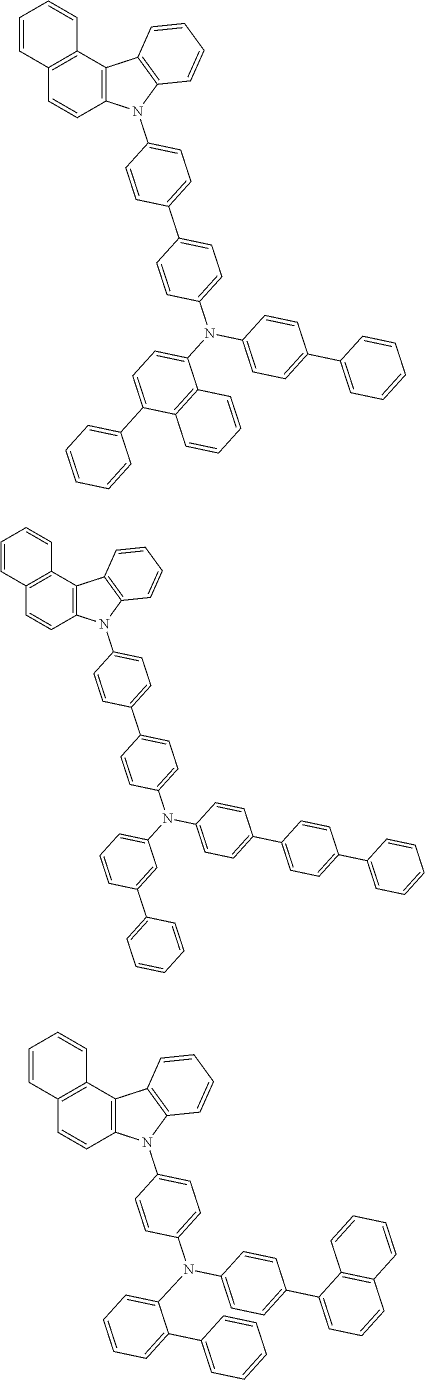 [SMILES: c1ccc(-c2ccc(N(c3ccc(-c4ccc(-n5c6ccccc6c6c7ccccc7ccc65)cc4)cc3)c3ccc(-c4ccccc4)c4ccccc34)cc2)cc1 ; c1ccc(-c2ccc(-c3ccc(N(c4ccc(-c5ccc(-n6c7ccccc7c7c8ccccc8ccc76)cc5)cc4)c4cccc(-c5ccccc5)c4)cc3)cc2)cc1 ; c1ccc(-c2ccccc2N(c2ccc(-c3cccc4ccccc34)cc2)c2ccc(-n3c4ccccc4c4c5ccccc5ccc43)cc2)cc1] 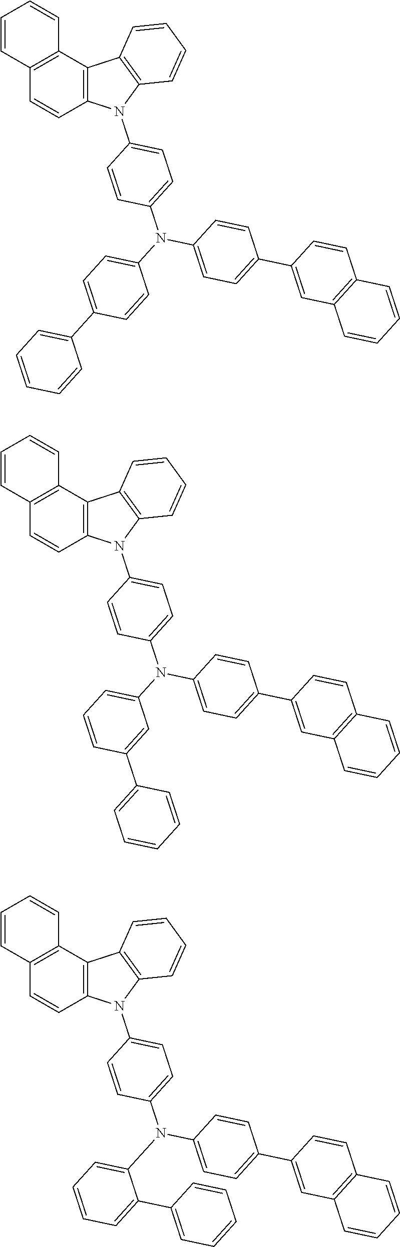 [SMILES: c1ccc(-c2ccc(N(c3ccc(-c4ccc5ccccc5c4)cc3)c3ccc(-n4c5ccccc5c5c6ccccc6ccc54)cc3)cc2)cc1 ; c1ccc(-c2cccc(N(c3ccc(-c4ccc5ccccc5c4)cc3)c3ccc(-n4c5ccccc5c5c6ccccc6ccc54)cc3)c2)cc1 ; c1ccc(-c2ccccc2N(c2ccc(-c3ccc4ccccc4c3)cc2)c2ccc(-n3c4ccccc4c4c5ccccc5ccc43)cc2)cc1] 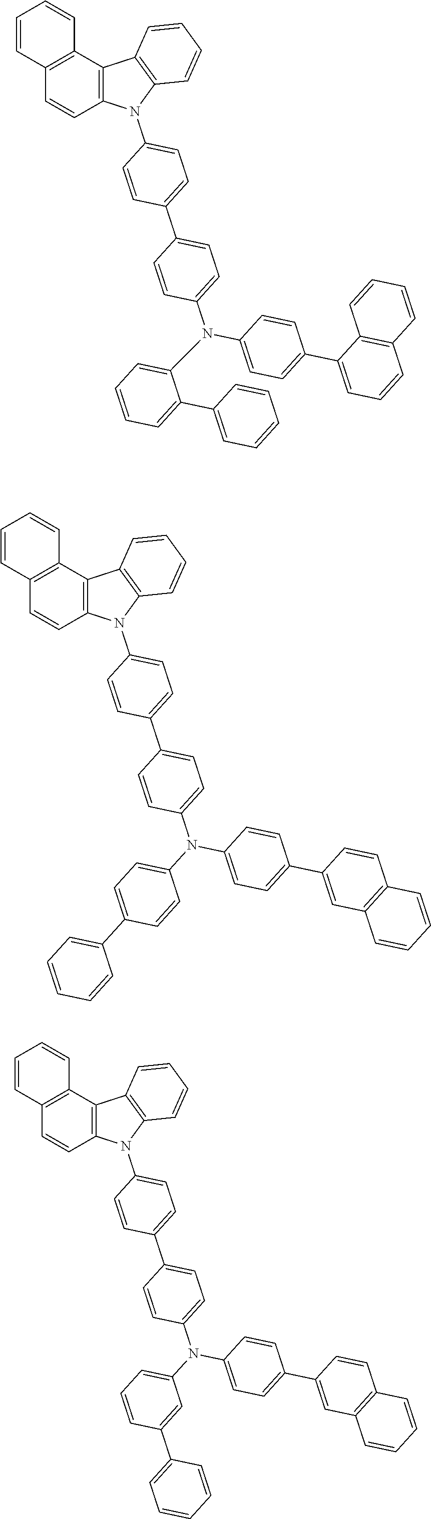 [SMILES: c1ccc(-c2ccccc2N(c2ccc(-c3ccc(-n4c5ccccc5c5c6ccccc6ccc54)cc3)cc2)c2ccc(-c3cccc4ccccc34)cc2)cc1 ; c1ccc(-c2ccc(N(c3ccc(-c4ccc(-n5c6ccccc6c6c7ccccc7ccc65)cc4)cc3)c3ccc(-c4ccc5ccccc5c4)cc3)cc2)cc1 ; c1ccc(-c2cccc(N(c3ccc(-c4ccc(-n5c6ccccc6c6c7ccccc7ccc65)cc4)cc3)c3ccc(-c4ccc5ccccc5c4)cc3)c2)cc1] 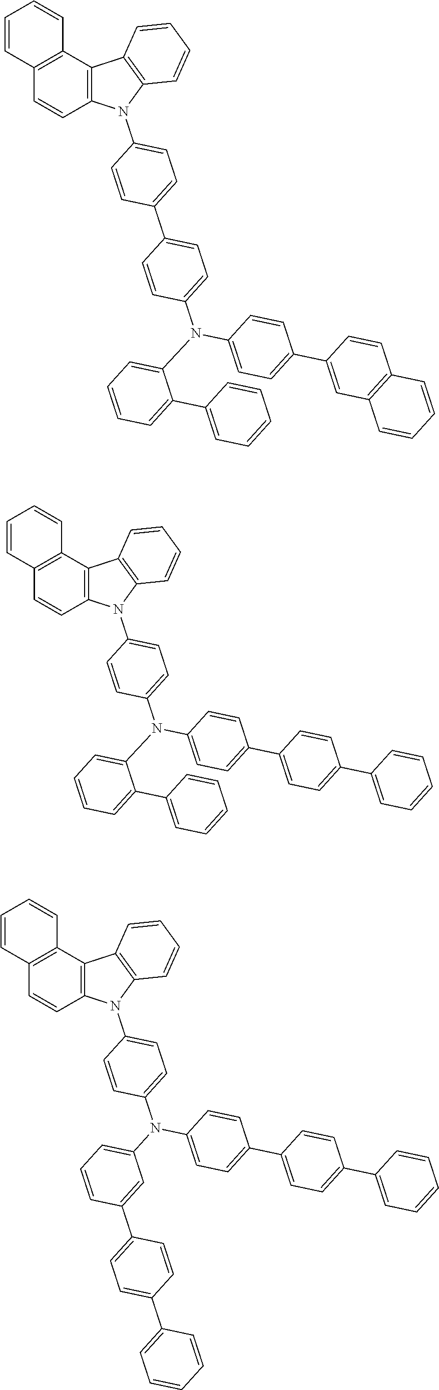 [SMILES: c1ccc(-c2ccccc2N(c2ccc(-c3ccc(-n4c5ccccc5c5c6ccccc6ccc54)cc3)cc2)c2ccc(-c3ccc4ccccc4c3)cc2)cc1 ; c1ccc(-c2ccc(-c3ccc(N(c4ccc(-n5c6ccccc6c6c7ccccc7ccc65)cc4)c4ccccc4-c4ccccc4)cc3)cc2)cc1 ; c1ccc(-c2ccc(-c3ccc(N(c4ccc(-n5c6ccccc6c6c7ccccc7ccc65)cc4)c4cccc(-c5ccc(-c6ccccc6)cc5)c4)cc3)cc2)cc1] 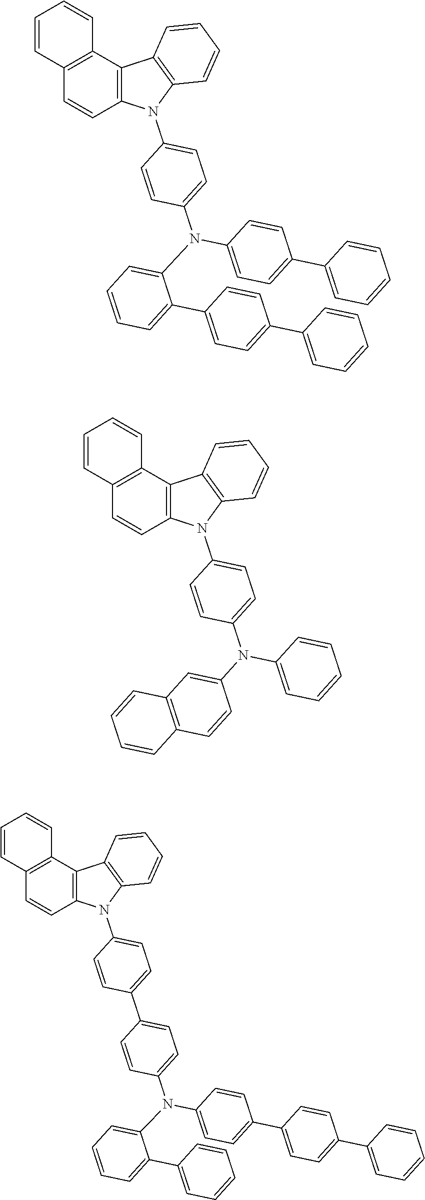 [SMILES: c1ccc(-c2ccc(-c3ccccc3N(c3ccc(-c4ccccc4)cc3)c3ccc(-n4c5ccccc5c5c6ccccc6ccc54)cc3)cc2)cc1 ; c1ccc(N(c2ccc(-n3c4ccccc4c4c5ccccc5ccc43)cc2)c2ccc3ccccc3c2)cc1 ; c1ccc(-c2ccc(-c3ccc(N(c4ccc(-c5ccc(-n6c7ccccc7c7c8ccccc8ccc76)cc5)cc4)c4ccccc4-c4ccccc4)cc3)cc2)cc1] 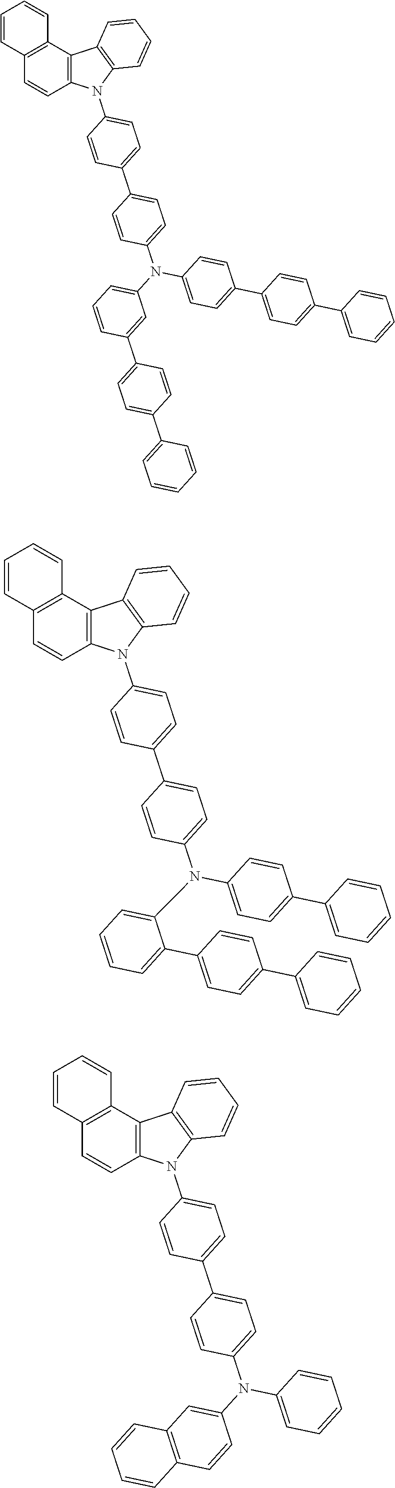 [SMILES: c1ccc(-c2ccc(-c3ccc(N(c4ccc(-c5ccc(-n6c7ccccc7c7c8ccccc8ccc76)cc5)cc4)c4cccc(-c5ccc(-c6ccccc6)cc5)c4)cc3)cc2)cc1 ; c1ccc(-c2ccc(-c3ccccc3N(c3ccc(-c4ccccc4)cc3)c3ccc(-c4ccc(-n5c6ccccc6c6c7ccccc7ccc65)cc4)cc3)cc2)cc1 ; c1ccc(N(c2ccc(-c3ccc(-n4c5ccccc5c5c6ccccc6ccc54)cc3)cc2)c2ccc3ccccc3c2)cc1] 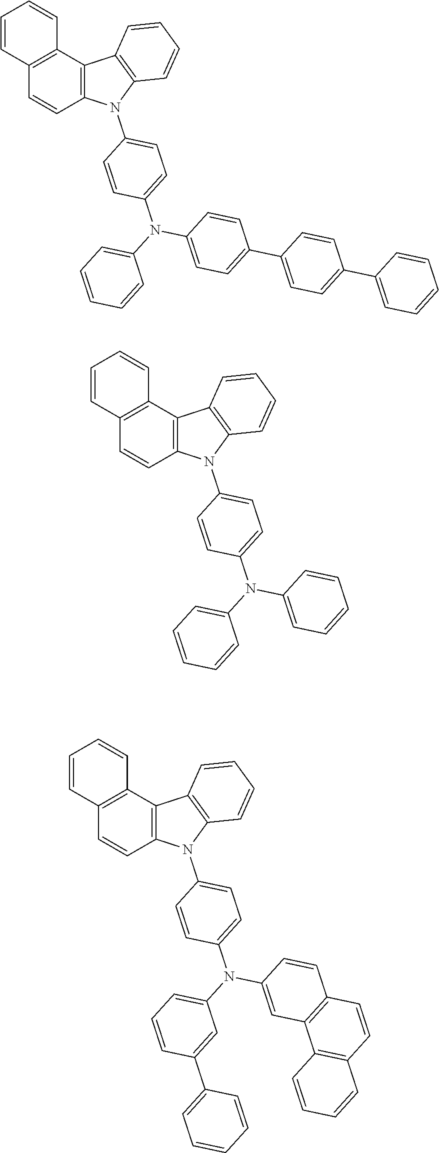 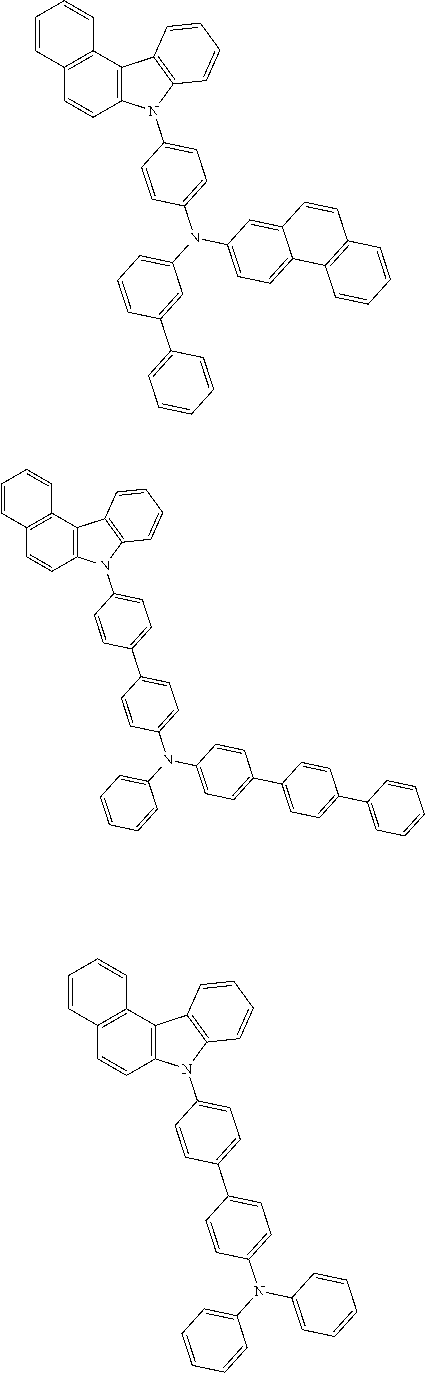 [SMILES: c1ccc(-c2cccc(N(c3ccc(-n4c5ccccc5c5c6ccccc6ccc54)cc3)c3ccc4c(ccc5ccccc54)c3)c2)cc1 ; c1ccc(-c2ccc(-c3ccc(N(c4ccccc4)c4ccc(-c5ccc(-n6c7ccccc7c7c8ccccc8ccc76)cc5)cc4)cc3)cc2)cc1 ; c1ccc(N(c2ccccc2)c2ccc(-c3ccc(-n4c5ccccc5c5c6ccccc6ccc54)cc3)cc2)cc1] 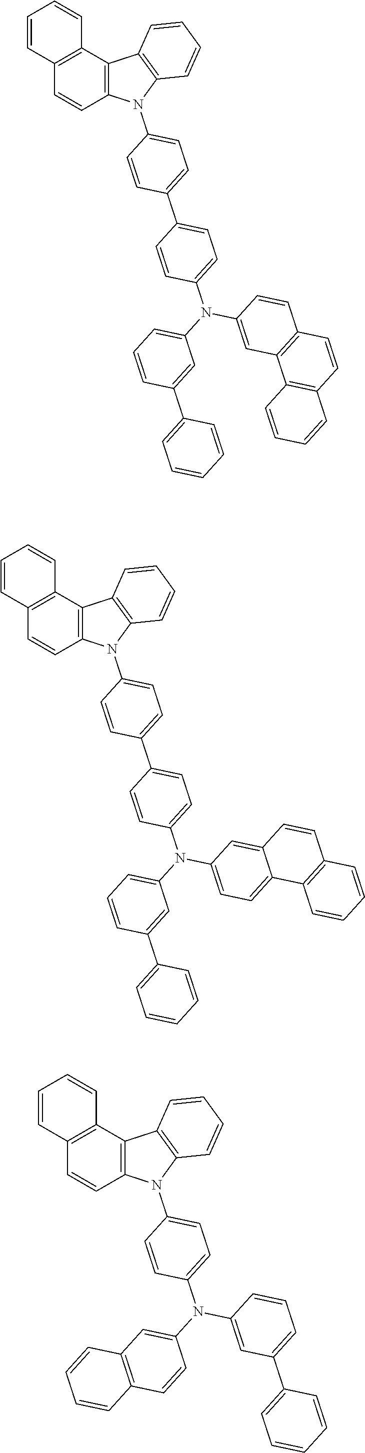 [SMILES: c1ccc(-c2cccc(N(c3ccc(-c4ccc(-n5c6ccccc6c6c7ccccc7ccc65)cc4)cc3)c3ccc4ccc5ccccc5c4c3)c2)cc1 ; c1ccc(-c2cccc(N(c3ccc(-c4ccc(-n5c6ccccc6c6c7ccccc7ccc65)cc4)cc3)c3ccc4c(ccc5ccccc54)c3)c2)cc1 ; c1ccc(-c2cccc(N(c3ccc(-n4c5ccccc5c5c6ccccc6ccc54)cc3)c3ccc4ccccc4c3)c2)cc1] 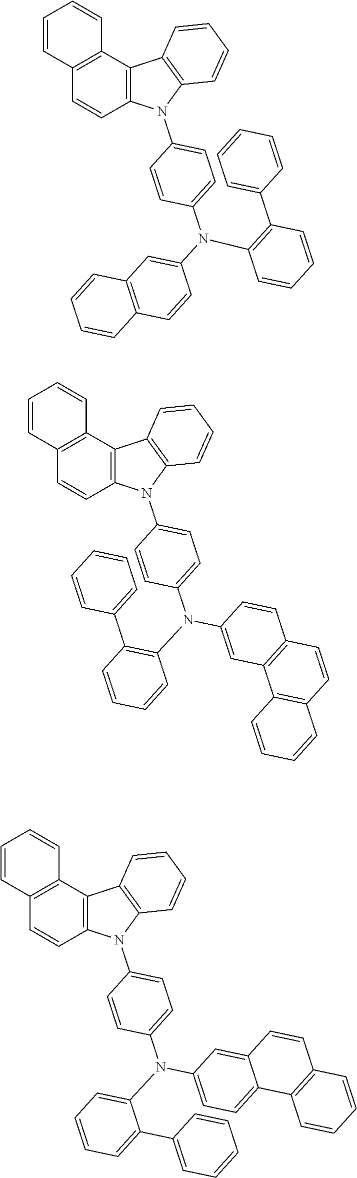 [SMILES: c1ccc(-c2ccccc2N(c2ccc(-n3c4ccccc4c4c5ccccc5ccc43)cc2)c2ccc3ccccc3c2)cc1 ; c1ccc(-c2ccccc2N(c2ccc(-n3c4ccccc4c4c5ccccc5ccc43)cc2)c2ccc3ccc4ccccc4c3c2)cc1 ; c1ccc(-c2ccccc2N(c2ccc(-n3c4ccccc4c4c5ccccc5ccc43)cc2)c2ccc3c(ccc4ccccc43)c2)cc1] 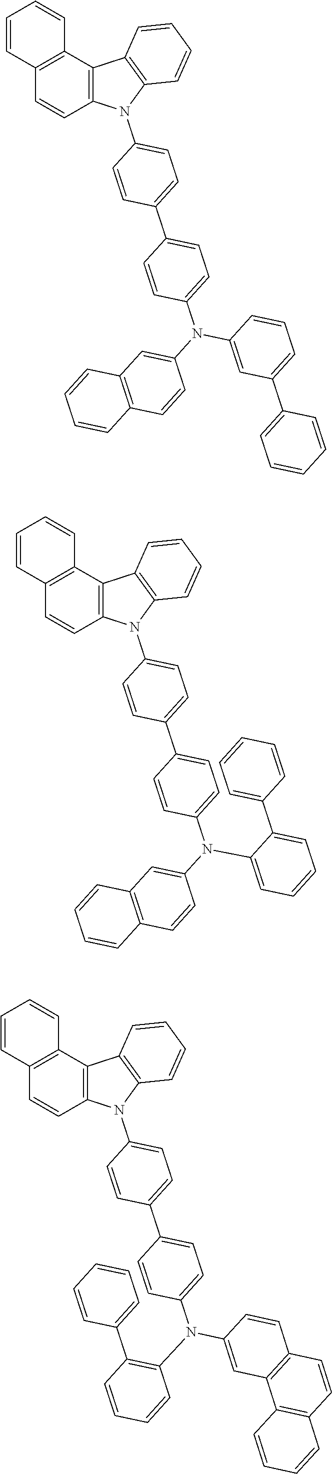 [SMILES: c1ccc(-c2cccc(N(c3ccc(-c4ccc(-n5c6ccccc6c6c7ccccc7ccc65)cc4)cc3)c3ccc4ccccc4c3)c2)cc1 ; c1ccc(-c2ccccc2N(c2ccc(-c3ccc(-n4c5ccccc5c5c6ccccc6ccc54)cc3)cc2)c2ccc3ccccc3c2)cc1 ; c1ccc(-c2ccccc2N(c2ccc(-c3ccc(-n4c5ccccc5c5c6ccccc6ccc54)cc3)cc2)c2ccc3ccc4ccccc4c3c2)cc1] 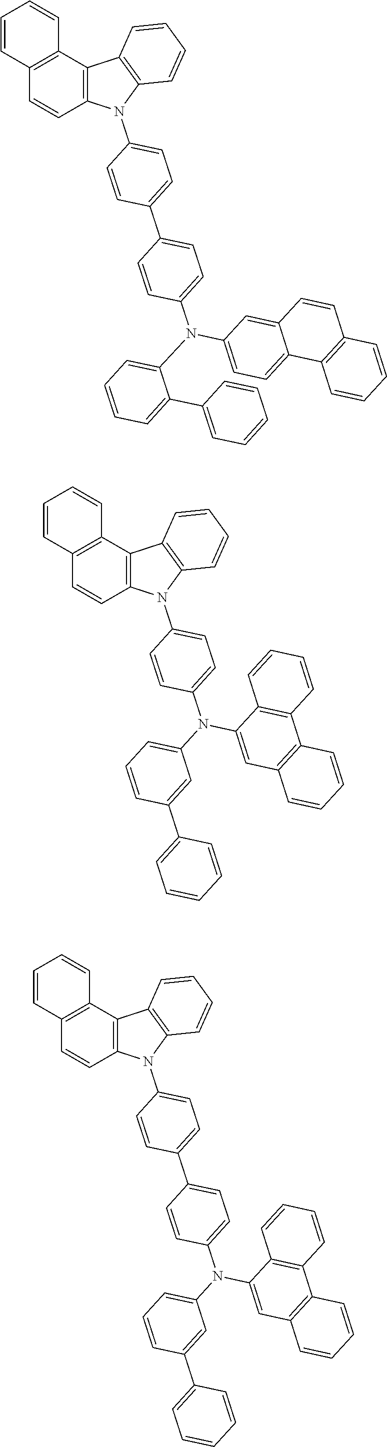 [SMILES: c1ccc(-c2ccccc2N(c2ccc(-c3ccc(-n4c5ccccc5c5c6ccccc6ccc54)cc3)cc2)c2ccc3c(ccc4ccccc43)c2)cc1 ; c1ccc(-c2cccc(N(c3ccc(-n4c5ccccc5c5c6ccccc6ccc54)cc3)c3cc4ccccc4c4ccccc34)c2)cc1 ; c1ccc(-c2cccc(N(c3ccc(-c4ccc(-n5c6ccccc6c6c7ccccc7ccc65)cc4)cc3)c3cc4ccccc4c4ccccc34)c2)cc1] 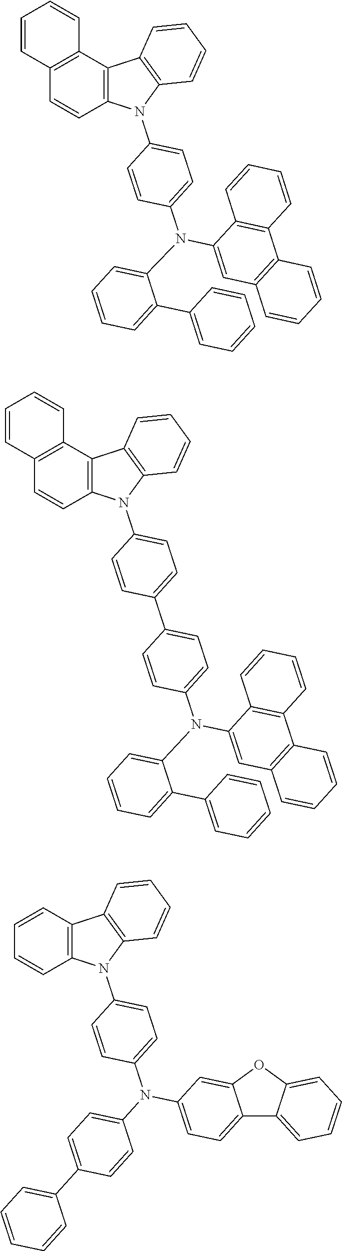 [SMILES: c1ccc(-c2ccccc2N(c2ccc(-n3c4ccccc4c4c5ccccc5ccc43)cc2)c2cc3ccccc3c3ccccc23)cc1 ; c1ccc(-c2ccccc2N(c2ccc(-c3ccc(-n4c5ccccc5c5c6ccccc6ccc54)cc3)cc2)c2cc3ccccc3c3ccccc23)cc1 ; c1ccc(-c2ccc(N(c3ccc(-n4c5ccccc5c5ccccc54)cc3)c3ccc4c(c3)oc3ccccc34)cc2)cc1] 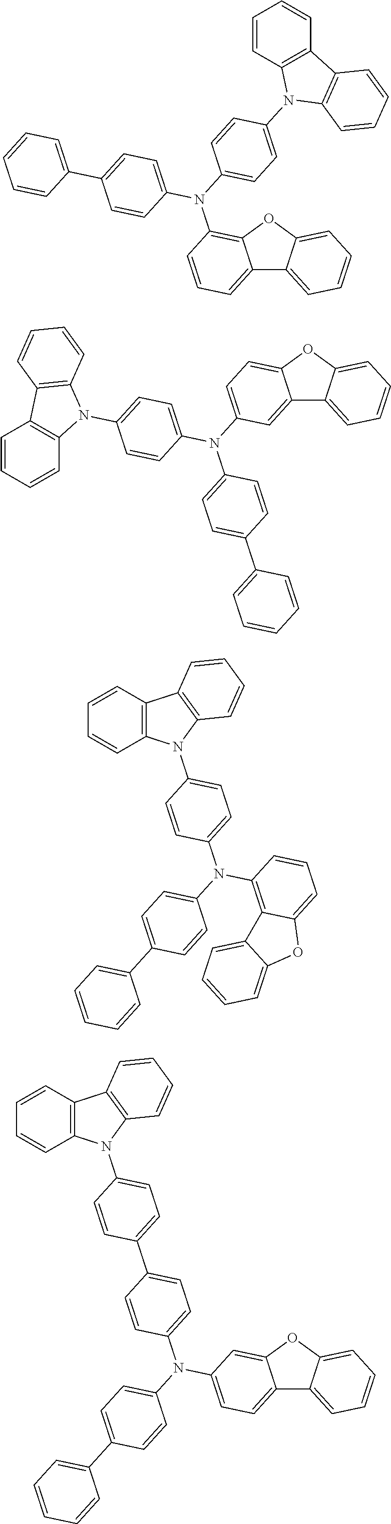 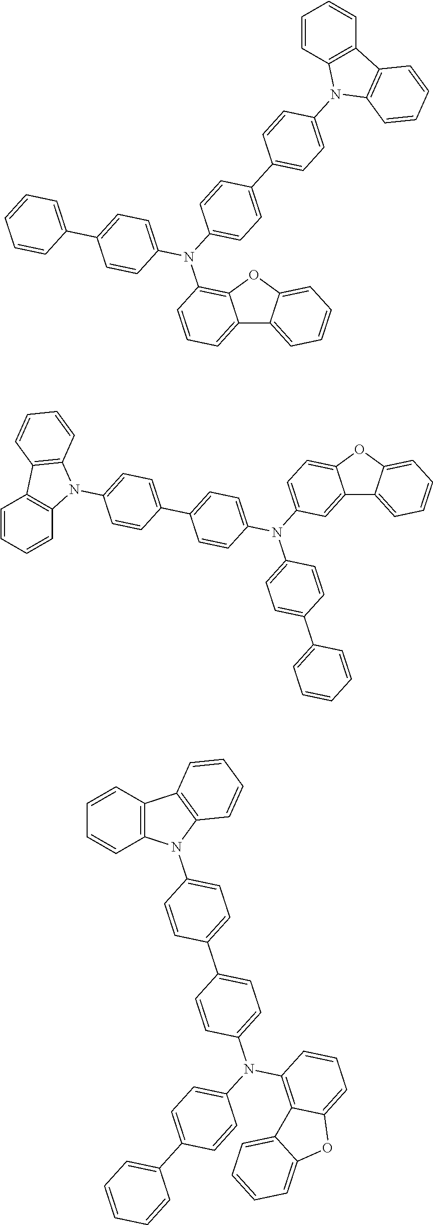 [SMILES: c1ccc(-c2ccc(N(c3ccc(-c4ccc(-n5c6ccccc6c6ccccc65)cc4)cc3)c3cccc4c3oc3ccccc34)cc2)cc1 ; c1ccc(-c2ccc(N(c3ccc(-c4ccc(-n5c6ccccc6c6ccccc65)cc4)cc3)c3ccc4oc5ccccc5c4c3)cc2)cc1 ; c1ccc(-c2ccc(N(c3ccc(-c4ccc(-n5c6ccccc6c6ccccc65)cc4)cc3)c3cccc4oc5ccccc5c34)cc2)cc1] 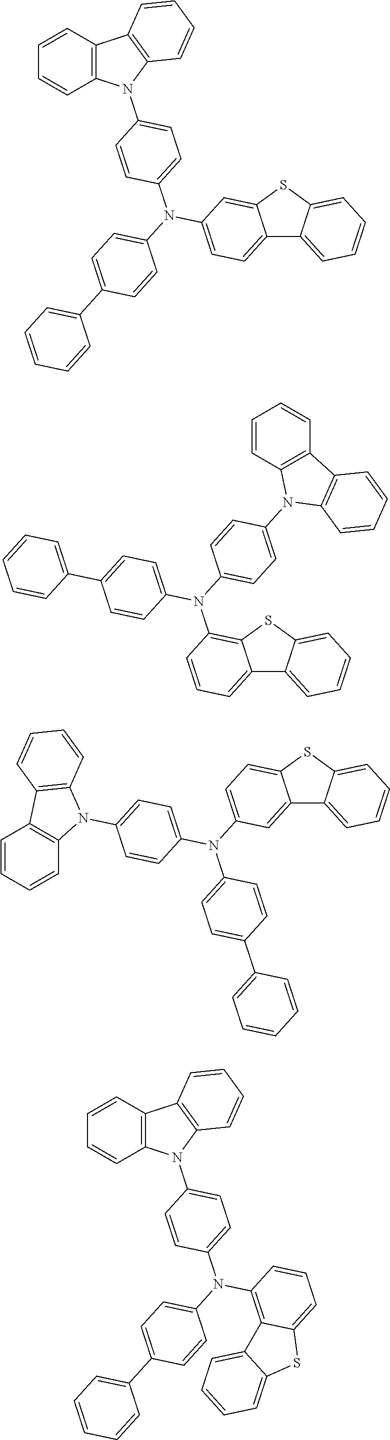 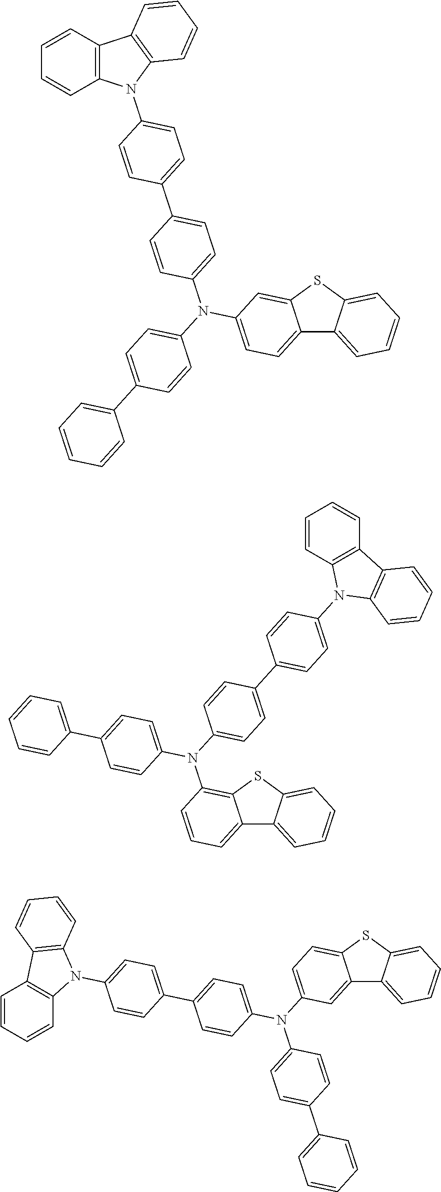 [SMILES: c1ccc(-c2ccc(N(c3ccc(-c4ccc(-n5c6ccccc6c6ccccc65)cc4)cc3)c3ccc4c(c3)sc3ccccc34)cc2)cc1 ; c1ccc(-c2ccc(N(c3ccc(-c4ccc(-n5c6ccccc6c6ccccc65)cc4)cc3)c3cccc4c3sc3ccccc34)cc2)cc1 ; c1ccc(-c2ccc(N(c3ccc(-c4ccc(-n5c6ccccc6c6ccccc65)cc4)cc3)c3ccc4sc5ccccc5c4c3)cc2)cc1] 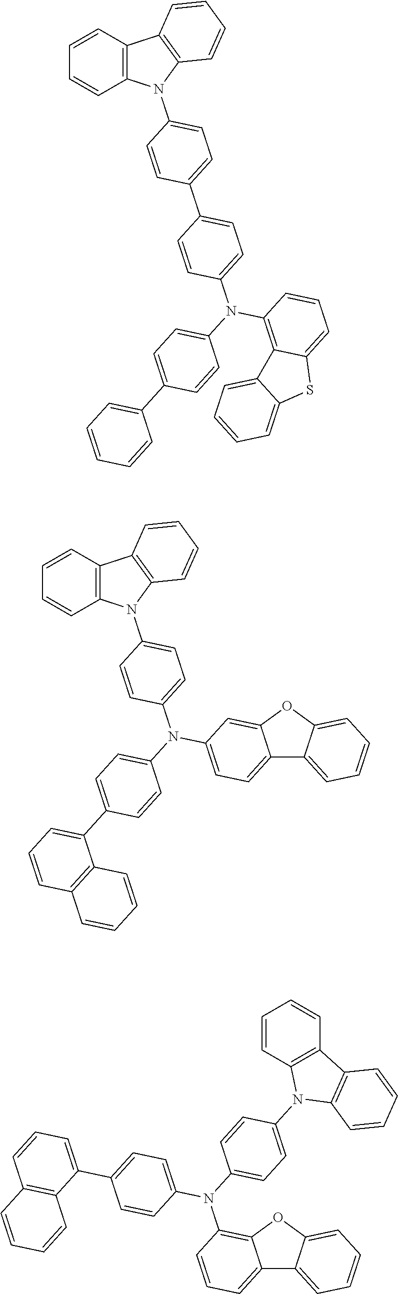 [SMILES: c1ccc(-c2ccc(N(c3ccc(-c4ccc(-n5c6ccccc6c6ccccc65)cc4)cc3)c3cccc4sc5ccccc5c34)cc2)cc1 ; c1ccc2c(-c3ccc(N(c4ccc(-n5c6ccccc6c6ccccc65)cc4)c4ccc5c(c4)oc4ccccc45)cc3)cccc2c1 ; c1ccc2c(-c3ccc(N(c4ccc(-n5c6ccccc6c6ccccc65)cc4)c4cccc5c4oc4ccccc45)cc3)cccc2c1] 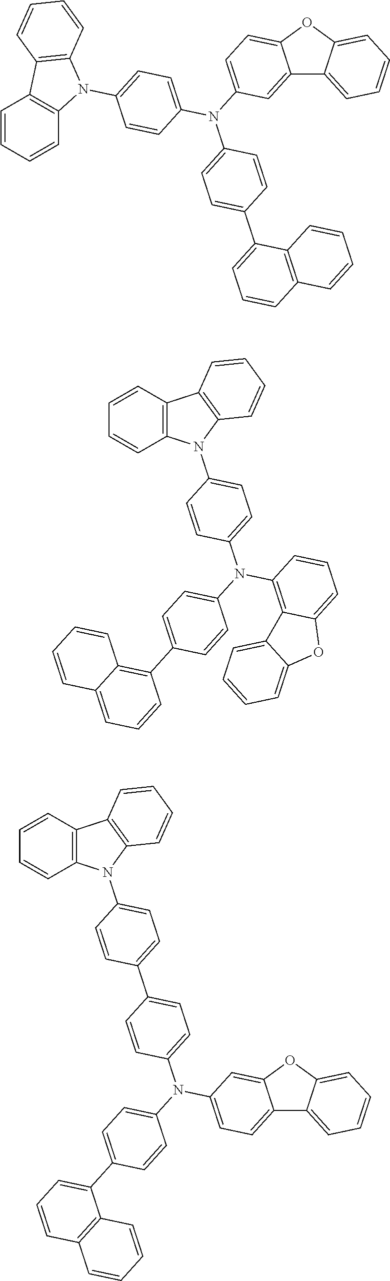 [SMILES: c1ccc2c(-c3ccc(N(c4ccc(-n5c6ccccc6c6ccccc65)cc4)c4ccc5oc6ccccc6c5c4)cc3)cccc2c1 ; c1ccc2c(-c3ccc(N(c4ccc(-n5c6ccccc6c6ccccc65)cc4)c4cccc5oc6ccccc6c45)cc3)cccc2c1 ; c1ccc2c(-c3ccc(N(c4ccc(-c5ccc(-n6c7ccccc7c7ccccc76)cc5)cc4)c4ccc5c(c4)oc4ccccc45)cc3)cccc2c1] 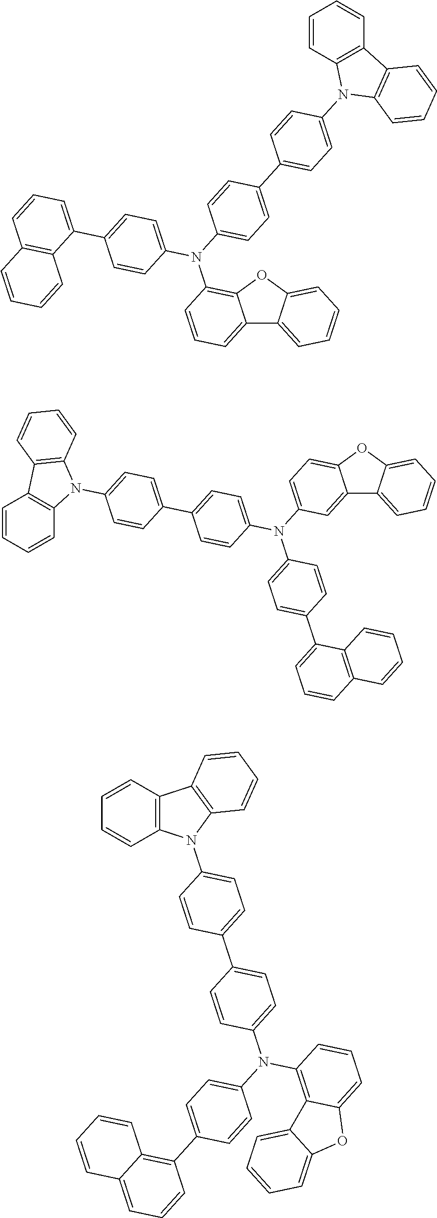 [SMILES: c1ccc2c(-c3ccc(N(c4ccc(-c5ccc(-n6c7ccccc7c7ccccc76)cc5)cc4)c4cccc5c4oc4ccccc45)cc3)cccc2c1 ; c1ccc2c(-c3ccc(N(c4ccc(-c5ccc(-n6c7ccccc7c7ccccc76)cc5)cc4)c4ccc5oc6ccccc6c5c4)cc3)cccc2c1 ; c1ccc2c(-c3ccc(N(c4ccc(-c5ccc(-n6c7ccccc7c7ccccc76)cc5)cc4)c4cccc5oc6ccccc6c45)cc3)cccc2c1] 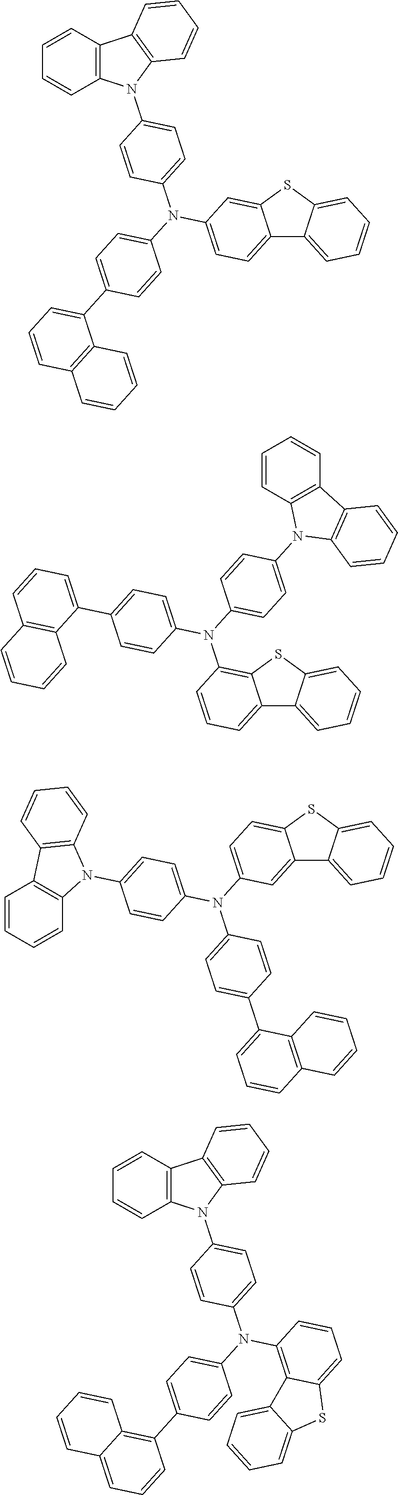 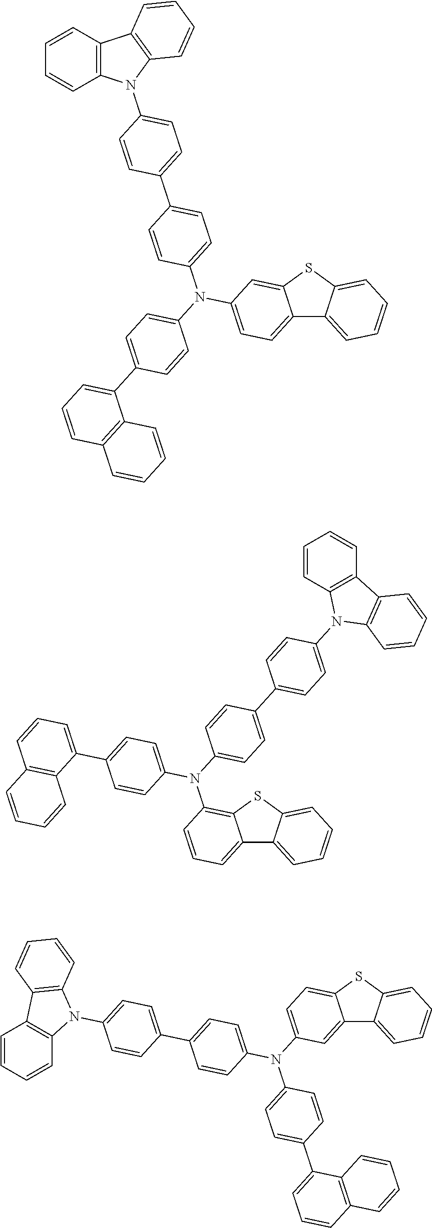 [SMILES: c1ccc2c(-c3ccc(N(c4ccc(-c5ccc(-n6c7ccccc7c7ccccc76)cc5)cc4)c4ccc5c(c4)sc4ccccc45)cc3)cccc2c1 ; c1ccc2c(-c3ccc(N(c4ccc(-c5ccc(-n6c7ccccc7c7ccccc76)cc5)cc4)c4cccc5c4sc4ccccc45)cc3)cccc2c1 ; c1ccc2c(-c3ccc(N(c4ccc(-c5ccc(-n6c7ccccc7c7ccccc76)cc5)cc4)c4ccc5sc6ccccc6c5c4)cc3)cccc2c1] 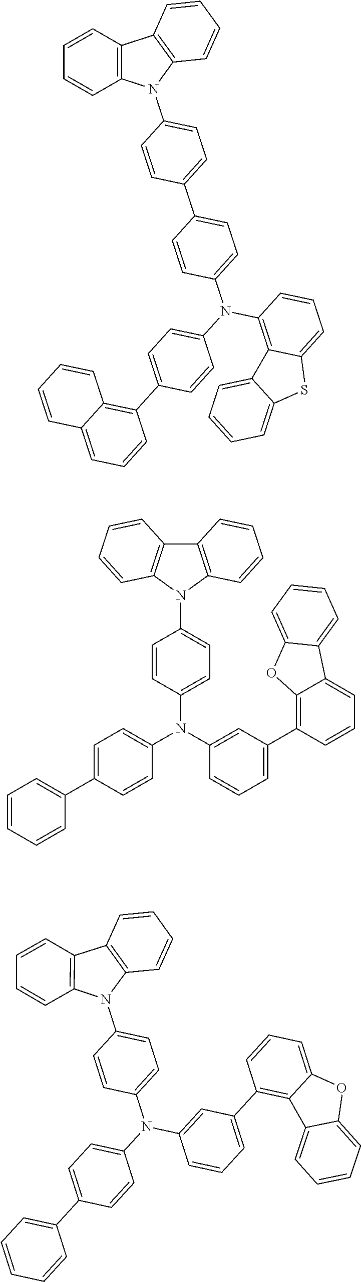 [SMILES: c1ccc2c(-c3ccc(N(c4ccc(-c5ccc(-n6c7ccccc7c7ccccc76)cc5)cc4)c4cccc5sc6ccccc6c45)cc3)cccc2c1 ; c1ccc(-c2ccc(N(c3ccc(-n4c5ccccc5c5ccccc54)cc3)c3cccc(-c4cccc5c4oc4ccccc45)c3)cc2)cc1 ; c1ccc(-c2ccc(N(c3ccc(-n4c5ccccc5c5ccccc54)cc3)c3cccc(-c4cccc5oc6ccccc6c45)c3)cc2)cc1] 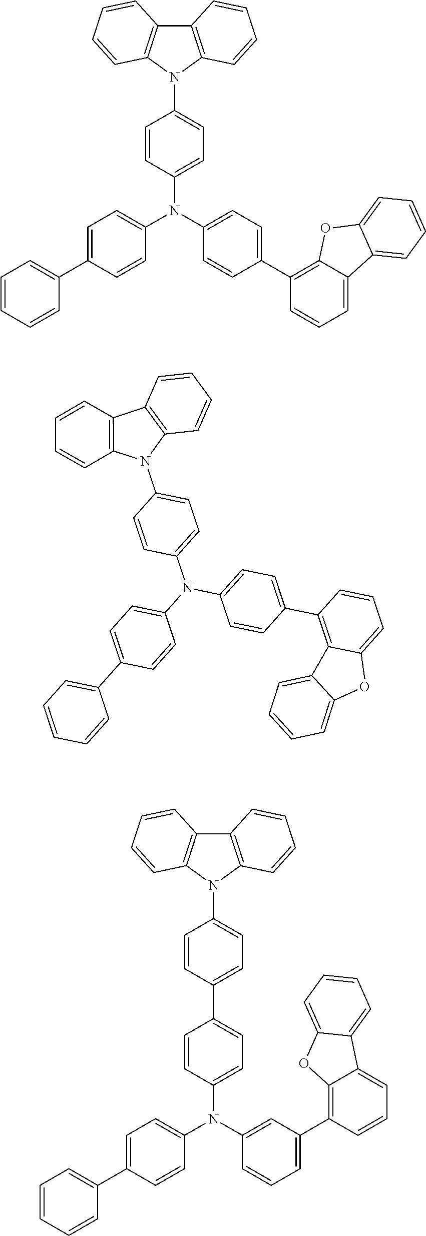 [SMILES: c1ccc(-c2ccc(N(c3ccc(-c4cccc5c4oc4ccccc45)cc3)c3ccc(-n4c5ccccc5c5ccccc54)cc3)cc2)cc1 ; c1ccc(-c2ccc(N(c3ccc(-c4cccc5oc6ccccc6c45)cc3)c3ccc(-n4c5ccccc5c5ccccc54)cc3)cc2)cc1 ; c1ccc(-c2ccc(N(c3ccc(-c4ccc(-n5c6ccccc6c6ccccc65)cc4)cc3)c3cccc(-c4cccc5c4oc4ccccc45)c3)cc2)cc1] 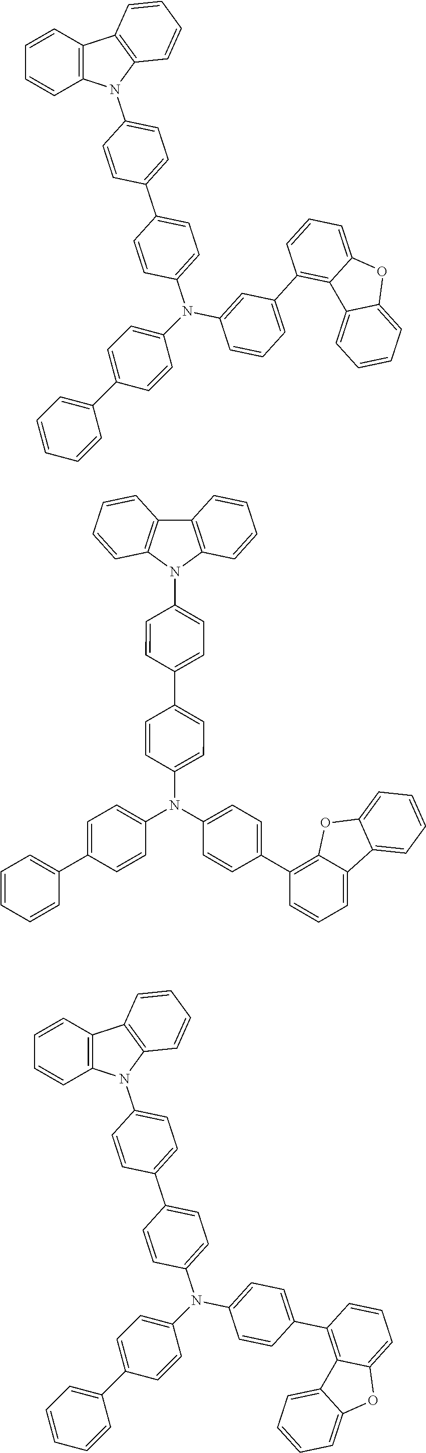 [SMILES: c1ccc(-c2ccc(N(c3ccc(-c4ccc(-n5c6ccccc6c6ccccc65)cc4)cc3)c3cccc(-c4cccc5oc6ccccc6c45)c3)cc2)cc1 ; c1ccc(-c2ccc(N(c3ccc(-c4ccc(-n5c6ccccc6c6ccccc65)cc4)cc3)c3ccc(-c4cccc5c4oc4ccccc45)cc3)cc2)cc1 ; c1ccc(-c2ccc(N(c3ccc(-c4ccc(-n5c6ccccc6c6ccccc65)cc4)cc3)c3ccc(-c4cccc5oc6ccccc6c45)cc3)cc2)cc1] 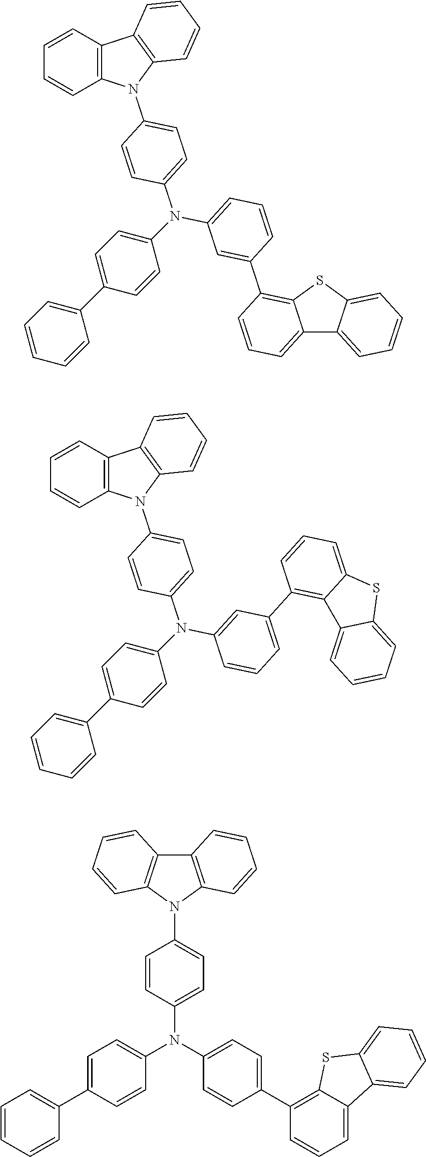 [SMILES: c1ccc(-c2ccc(N(c3ccc(-n4c5ccccc5c5ccccc54)cc3)c3cccc(-c4cccc5c4sc4ccccc45)c3)cc2)cc1 ; c1ccc(-c2ccc(N(c3ccc(-n4c5ccccc5c5ccccc54)cc3)c3cccc(-c4cccc5sc6ccccc6c45)c3)cc2)cc1 ; c1ccc(-c2ccc(N(c3ccc(-c4cccc5c4sc4ccccc45)cc3)c3ccc(-n4c5ccccc5c5ccccc54)cc3)cc2)cc1] 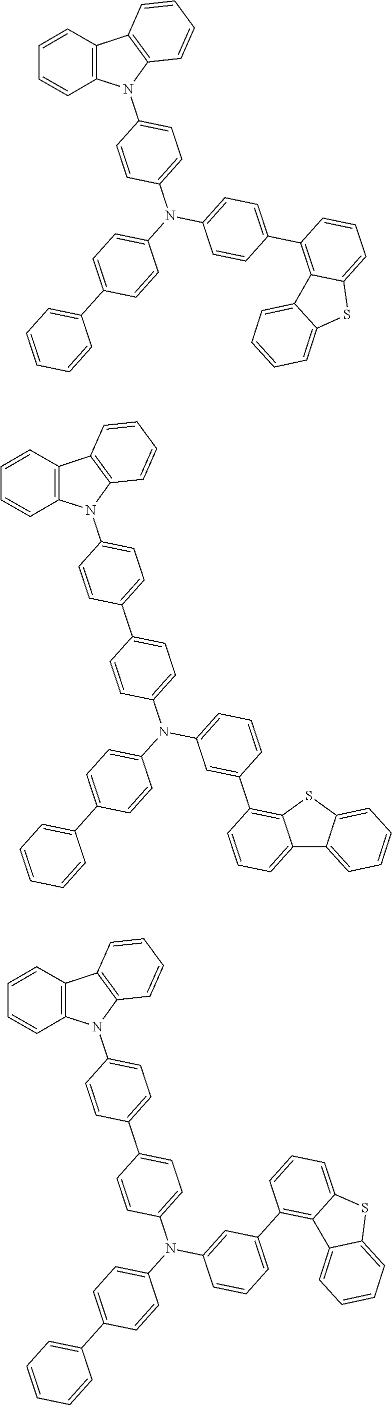 [SMILES: c1ccc(-c2ccc(N(c3ccc(-c4cccc5sc6ccccc6c45)cc3)c3ccc(-n4c5ccccc5c5ccccc54)cc3)cc2)cc1 ; c1ccc(-c2ccc(N(c3ccc(-c4ccc(-n5c6ccccc6c6ccccc65)cc4)cc3)c3cccc(-c4cccc5c4sc4ccccc45)c3)cc2)cc1 ; c1ccc(-c2ccc(N(c3ccc(-c4ccc(-n5c6ccccc6c6ccccc65)cc4)cc3)c3cccc(-c4cccc5sc6ccccc6c45)c3)cc2)cc1] 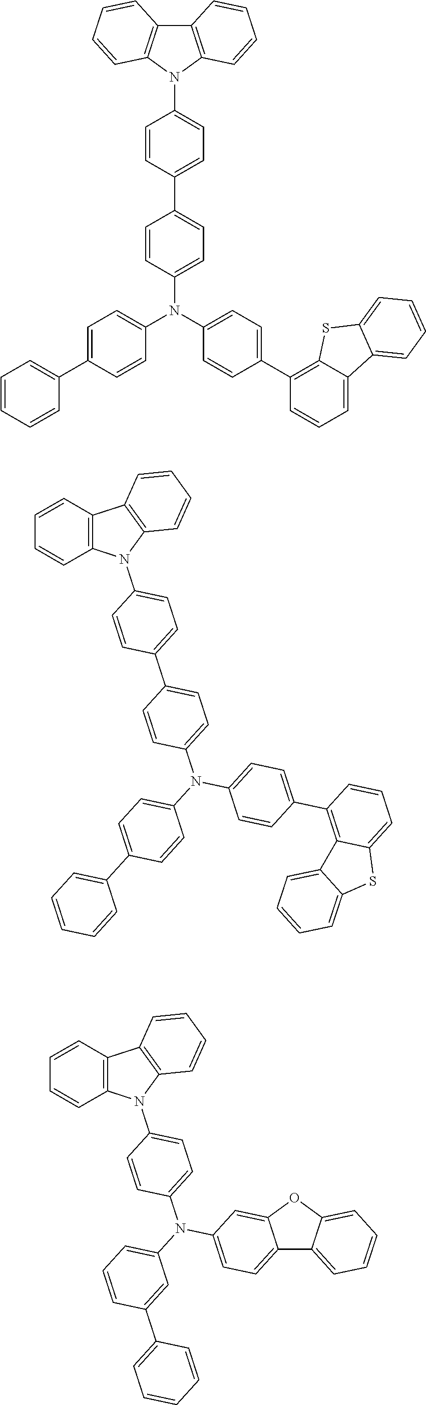 [SMILES: c1ccc(-c2ccc(N(c3ccc(-c4ccc(-n5c6ccccc6c6ccccc65)cc4)cc3)c3ccc(-c4cccc5c4sc4ccccc45)cc3)cc2)cc1 ; c1ccc(-c2ccc(N(c3ccc(-c4ccc(-n5c6ccccc6c6ccccc65)cc4)cc3)c3ccc(-c4cccc5sc6ccccc6c45)cc3)cc2)cc1 ; c1ccc(-c2cccc(N(c3ccc(-n4c5ccccc5c5ccccc54)cc3)c3ccc4c(c3)oc3ccccc34)c2)cc1] 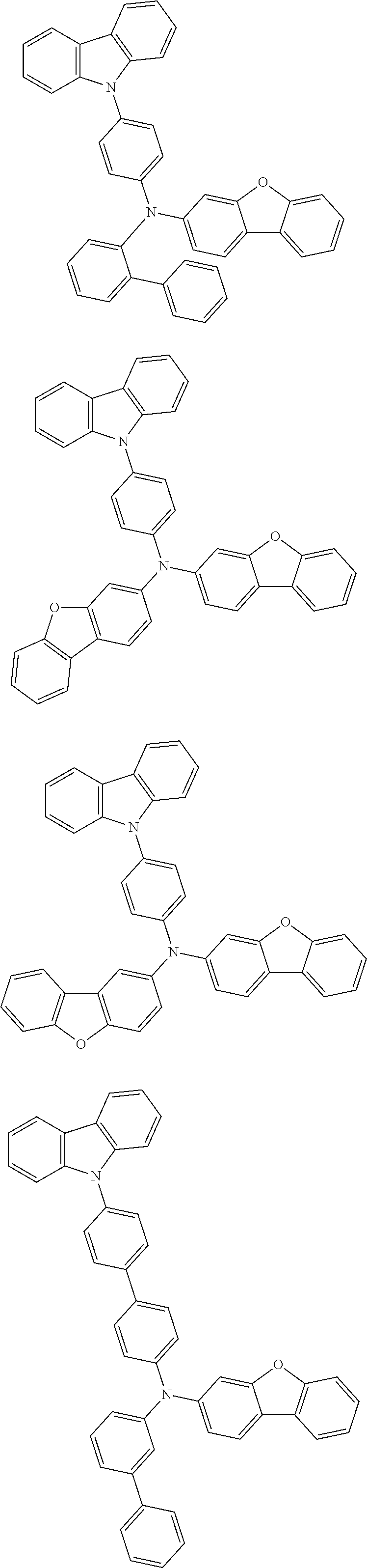 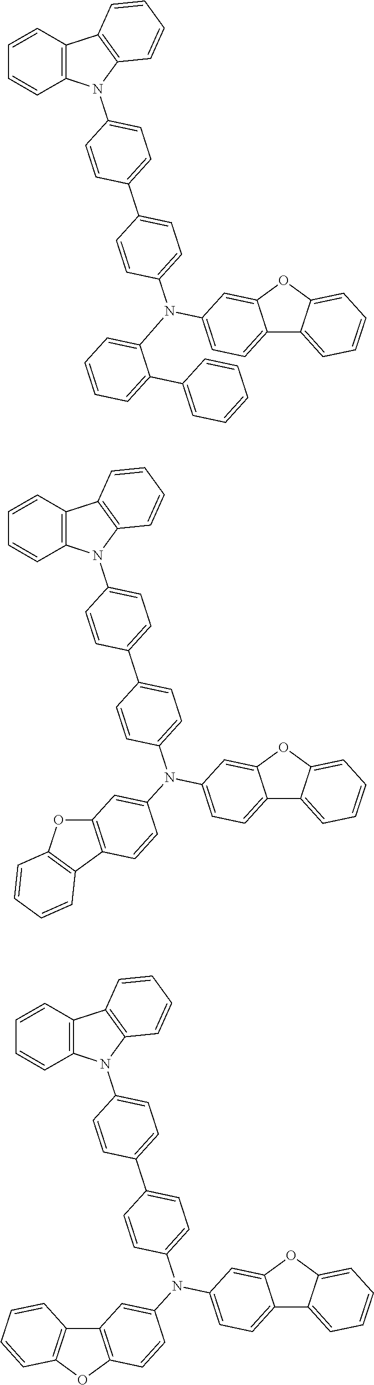 [SMILES: c1ccc(-c2ccccc2N(c2ccc(-c3ccc(-n4c5ccccc5c5ccccc54)cc3)cc2)c2ccc3c(c2)oc2ccccc23)cc1 ; c1ccc2c(c1)oc1cc(N(c3ccc(-c4ccc(-n5c6ccccc6c6ccccc65)cc4)cc3)c3ccc4c(c3)oc3ccccc34)ccc12 ; c1ccc2c(c1)oc1cc(N(c3ccc(-c4ccc(-n5c6ccccc6c6ccccc65)cc4)cc3)c3ccc4oc5ccccc5c4c3)ccc12] 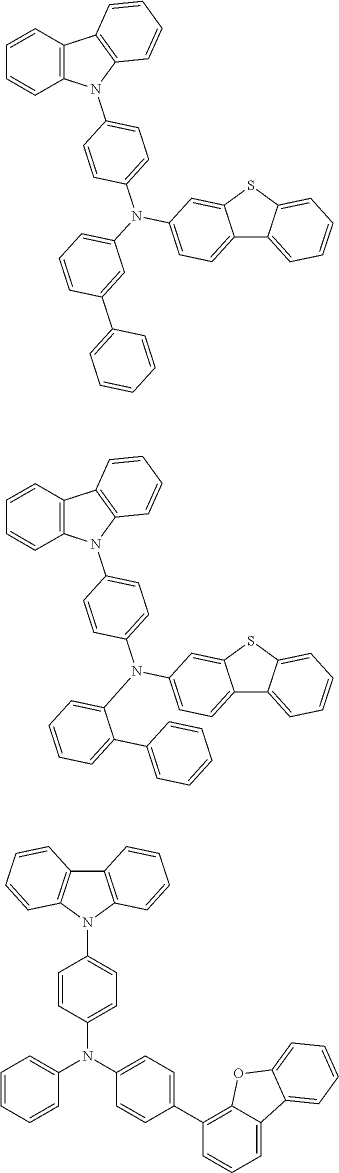 [SMILES: c1ccc(-c2cccc(N(c3ccc(-n4c5ccccc5c5ccccc54)cc3)c3ccc4c(c3)sc3ccccc34)c2)cc1 ; c1ccc(-c2ccccc2N(c2ccc(-n3c4ccccc4c4ccccc43)cc2)c2ccc3c(c2)sc2ccccc23)cc1 ; c1ccc(N(c2ccc(-c3cccc4c3oc3ccccc34)cc2)c2ccc(-n3c4ccccc4c4ccccc43)cc2)cc1] 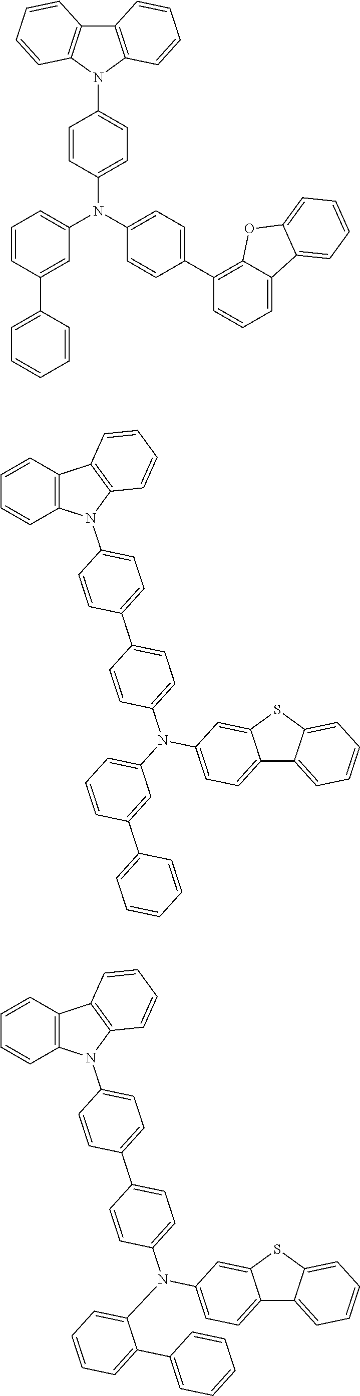 [SMILES: c1ccc(-c2cccc(N(c3ccc(-c4cccc5c4oc4ccccc45)cc3)c3ccc(-n4c5ccccc5c5ccccc54)cc3)c2)cc1 ; c1ccc(-c2cccc(N(c3ccc(-c4ccc(-n5c6ccccc6c6ccccc65)cc4)cc3)c3ccc4c(c3)sc3ccccc34)c2)cc1 ; c1ccc(-c2ccccc2N(c2ccc(-c3ccc(-n4c5ccccc5c5ccccc54)cc3)cc2)c2ccc3c(c2)sc2ccccc23)cc1] 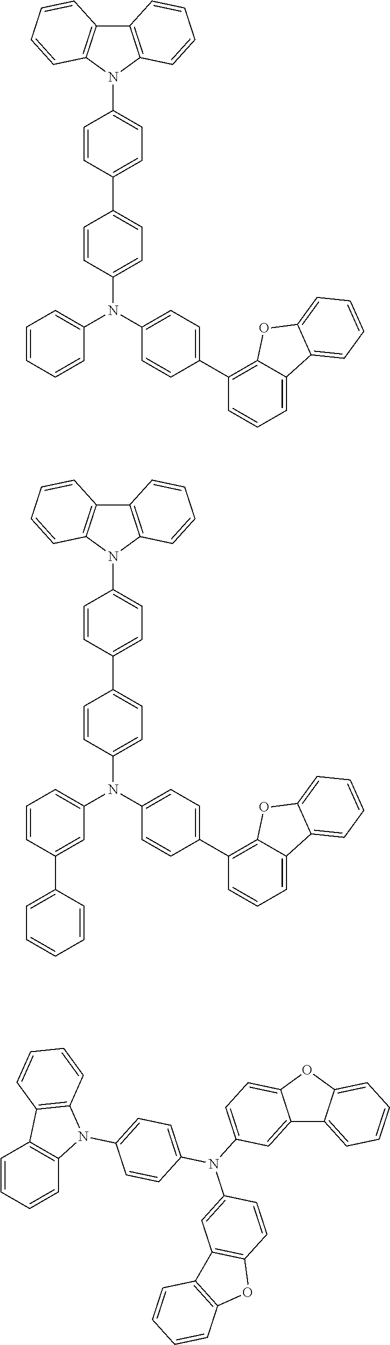 [SMILES: c1ccc(N(c2ccc(-c3ccc(-n4c5ccccc5c5ccccc54)cc3)cc2)c2ccc(-c3cccc4c3oc3ccccc34)cc2)cc1 ; c1ccc(-c2cccc(N(c3ccc(-c4ccc(-n5c6ccccc6c6ccccc65)cc4)cc3)c3ccc(-c4cccc5c4oc4ccccc45)cc3)c2)cc1 ; c1ccc2c(c1)oc1ccc(N(c3ccc(-n4c5ccccc5c5ccccc54)cc3)c3ccc4oc5ccccc5c4c3)cc12] 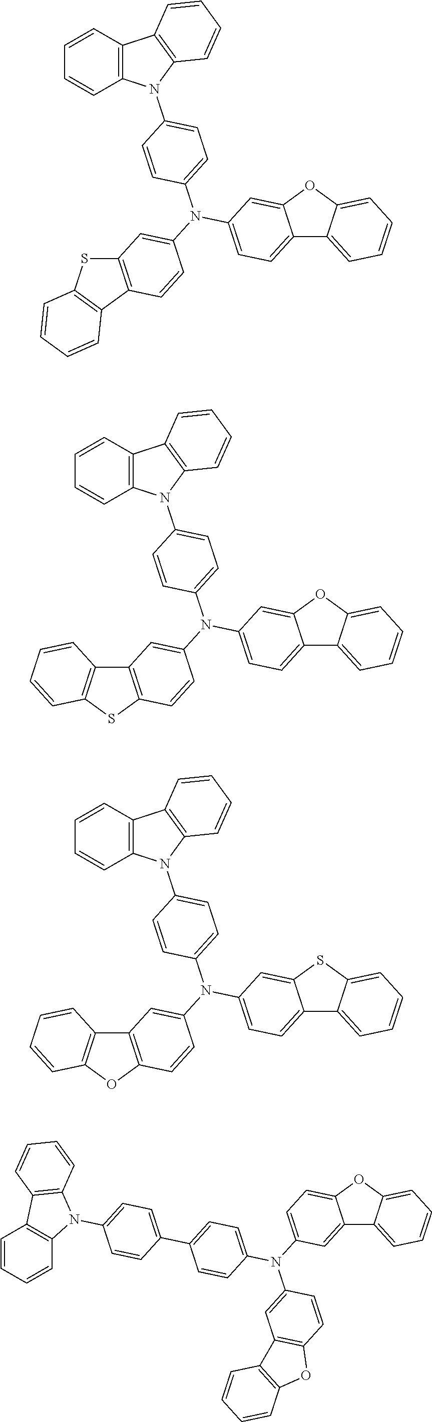 [SMILES: c1ccc2c(c1)oc1cc(N(c3ccc(-n4c5ccccc5c5ccccc54)cc3)c3ccc4c(c3)sc3ccccc34)ccc12 ; c1ccc2c(c1)oc1cc(N(c3ccc(-n4c5ccccc5c5ccccc54)cc3)c3ccc4sc5ccccc5c4c3)ccc12 ; c1ccc2c(c1)oc1ccc(N(c3ccc(-n4c5ccccc5c5ccccc54)cc3)c3ccc4c(c3)sc3ccccc34)cc12 ; c1ccc2c(c1)oc1ccc(N(c3ccc(-c4ccc(-n5c6ccccc6c6ccccc65)cc4)cc3)c3ccc4oc5ccccc5c4c3)cc12] 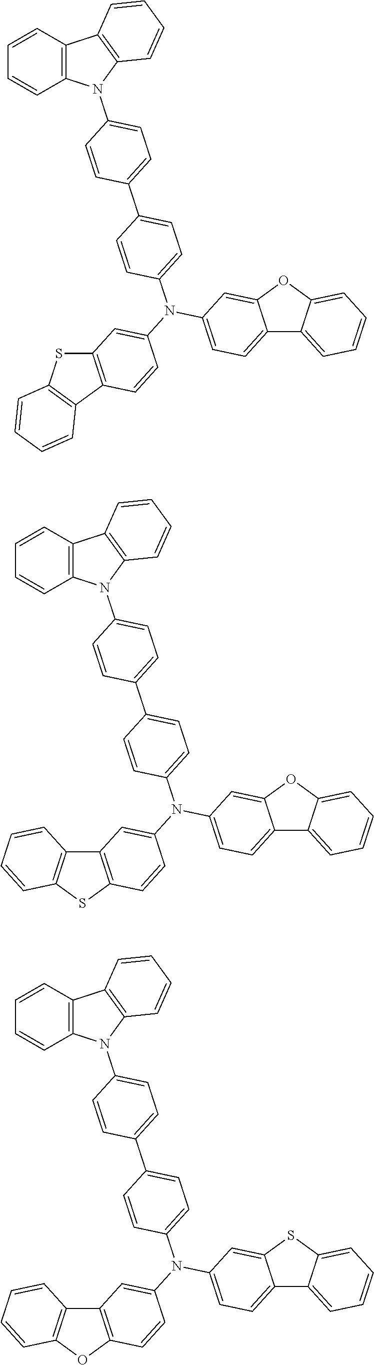 [SMILES: c1ccc2c(c1)oc1cc(N(c3ccc(-c4ccc(-n5c6ccccc6c6ccccc65)cc4)cc3)c3ccc4c(c3)sc3ccccc34)ccc12 ; c1ccc2c(c1)oc1cc(N(c3ccc(-c4ccc(-n5c6ccccc6c6ccccc65)cc4)cc3)c3ccc4sc5ccccc5c4c3)ccc12 ; c1ccc2c(c1)oc1ccc(N(c3ccc(-c4ccc(-n5c6ccccc6c6ccccc65)cc4)cc3)c3ccc4c(c3)sc3ccccc34)cc12] 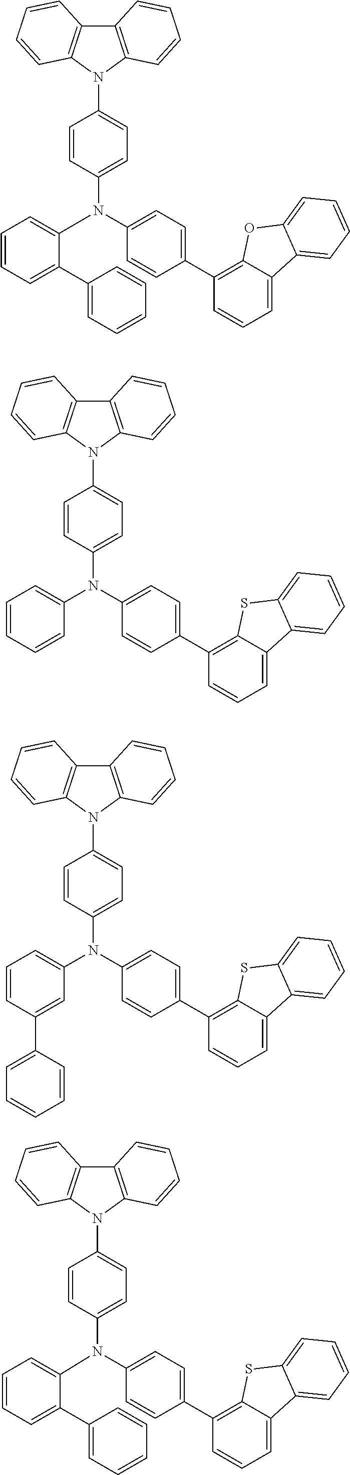 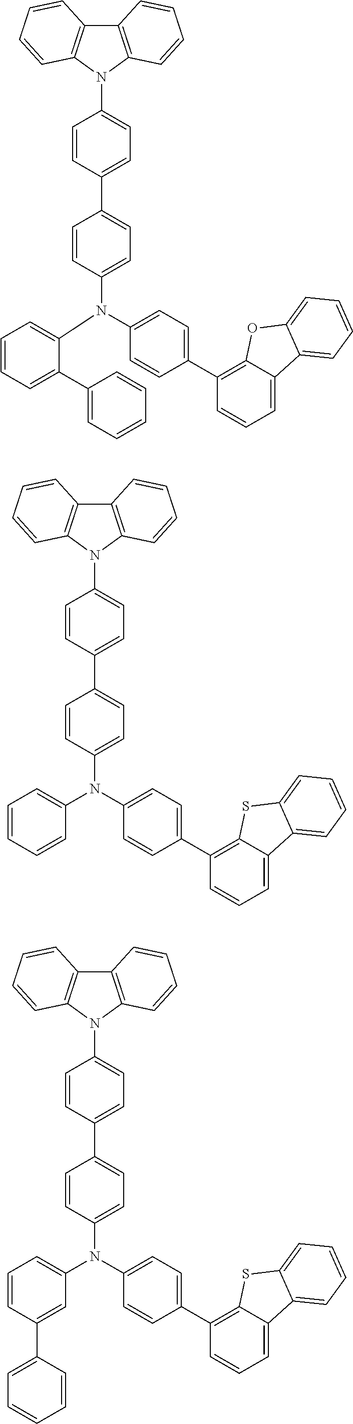 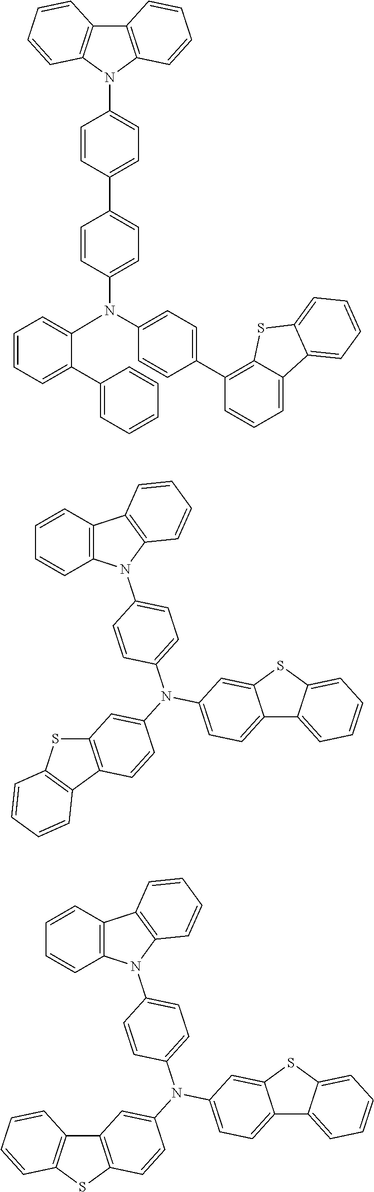 [SMILES: c1ccc(-c2ccccc2N(c2ccc(-c3ccc(-n4c5ccccc5c5ccccc54)cc3)cc2)c2ccc(-c3cccc4c3sc3ccccc34)cc2)cc1 ; c1ccc2c(c1)sc1cc(N(c3ccc(-n4c5ccccc5c5ccccc54)cc3)c3ccc4c(c3)sc3ccccc34)ccc12 ; c1ccc2c(c1)sc1cc(N(c3ccc(-n4c5ccccc5c5ccccc54)cc3)c3ccc4sc5ccccc5c4c3)ccc12] 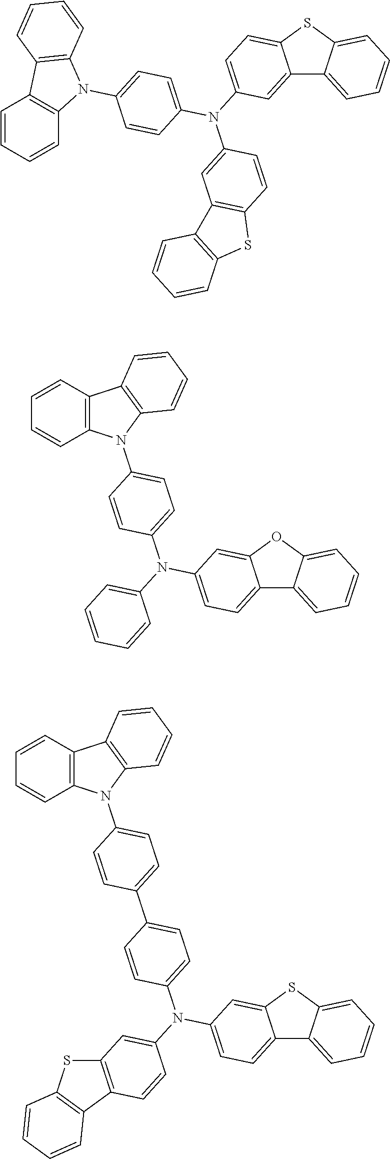 [SMILES: c1ccc2c(c1)sc1ccc(N(c3ccc(-n4c5ccccc5c5ccccc54)cc3)c3ccc4sc5ccccc5c4c3)cc12 ; c1ccc(N(c2ccc(-n3c4ccccc4c4ccccc43)cc2)c2ccc3c(c2)oc2ccccc23)cc1 ; c1ccc2c(c1)sc1cc(N(c3ccc(-c4ccc(-n5c6ccccc6c6ccccc65)cc4)cc3)c3ccc4c(c3)sc3ccccc34)ccc12] 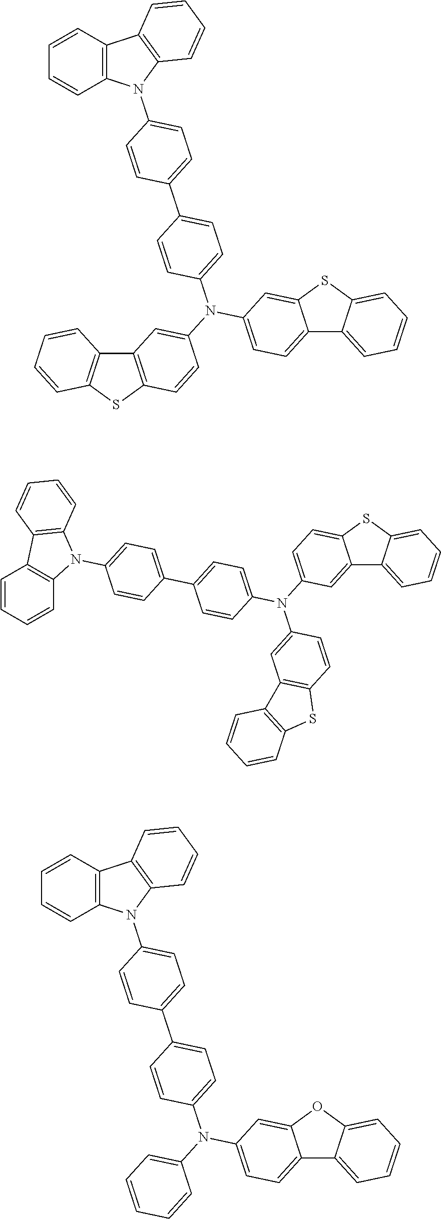 [SMILES: c1ccc2c(c1)sc1cc(N(c3ccc(-c4ccc(-n5c6ccccc6c6ccccc65)cc4)cc3)c3ccc4sc5ccccc5c4c3)ccc12 ; c1ccc2c(c1)sc1ccc(N(c3ccc(-c4ccc(-n5c6ccccc6c6ccccc65)cc4)cc3)c3ccc4sc5ccccc5c4c3)cc12 ; c1ccc(N(c2ccc(-c3ccc(-n4c5ccccc5c5ccccc54)cc3)cc2)c2ccc3c(c2)oc2ccccc23)cc1] 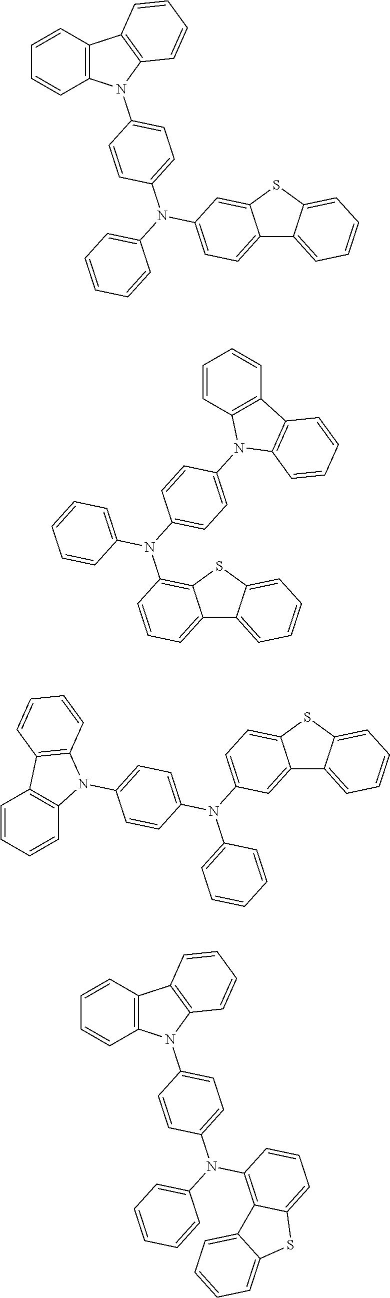 [SMILES: c1ccc(N(c2ccc(-n3c4ccccc4c4ccccc43)cc2)c2ccc3c(c2)sc2ccccc23)cc1 ; c1ccc(N(c2ccc(-n3c4ccccc4c4ccccc43)cc2)c2cccc3c2sc2ccccc23)cc1 ; c1ccc(N(c2ccc(-n3c4ccccc4c4ccccc43)cc2)c2ccc3sc4ccccc4c3c2)cc1 ; c1ccc(N(c2ccc(-n3c4ccccc4c4ccccc43)cc2)c2cccc3sc4ccccc4c23)cc1] 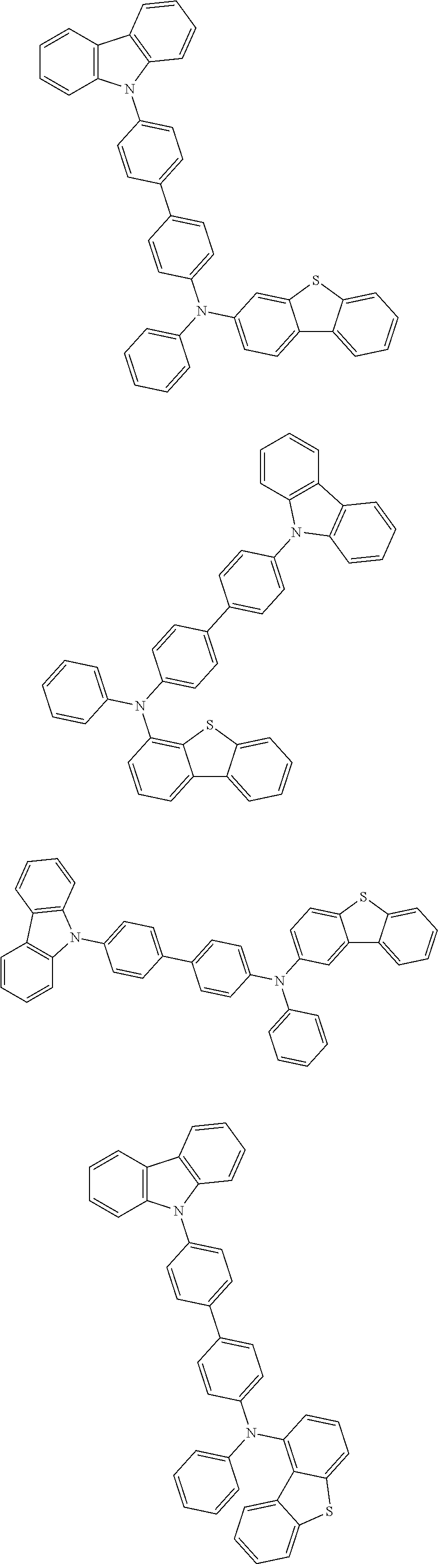 [SMILES: c1ccc(N(c2ccc(-c3ccc(-n4c5ccccc5c5ccccc54)cc3)cc2)c2ccc3c(c2)sc2ccccc23)cc1 ; c1ccc(N(c2ccc(-c3ccc(-n4c5ccccc5c5ccccc54)cc3)cc2)c2cccc3c2sc2ccccc23)cc1 ; c1ccc(N(c2ccc(-c3ccc(-n4c5ccccc5c5ccccc54)cc3)cc2)c2ccc3sc4ccccc4c3c2)cc1 ; c1ccc(N(c2ccc(-c3ccc(-n4c5ccccc5c5ccccc54)cc3)cc2)c2cccc3sc4ccccc4c23)cc1] 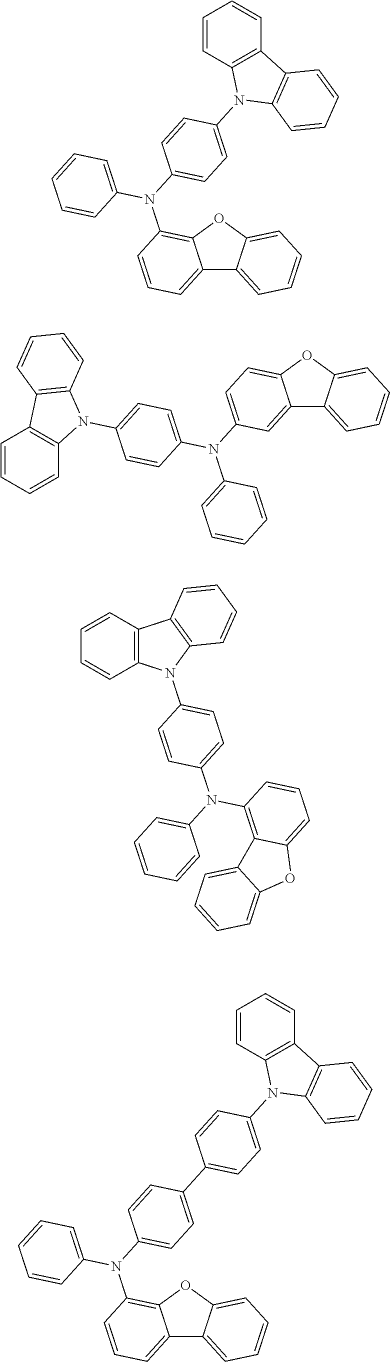 [SMILES: c1ccc(N(c2ccc(-n3c4ccccc4c4ccccc43)cc2)c2cccc3c2oc2ccccc23)cc1 ; c1ccc(N(c2ccc(-n3c4ccccc4c4ccccc43)cc2)c2ccc3oc4ccccc4c3c2)cc1 ; c1ccc(N(c2ccc(-n3c4ccccc4c4ccccc43)cc2)c2cccc3oc4ccccc4c23)cc1 ; c1ccc(N(c2ccc(-c3ccc(-n4c5ccccc5c5ccccc54)cc3)cc2)c2cccc3c2oc2ccccc23)cc1] 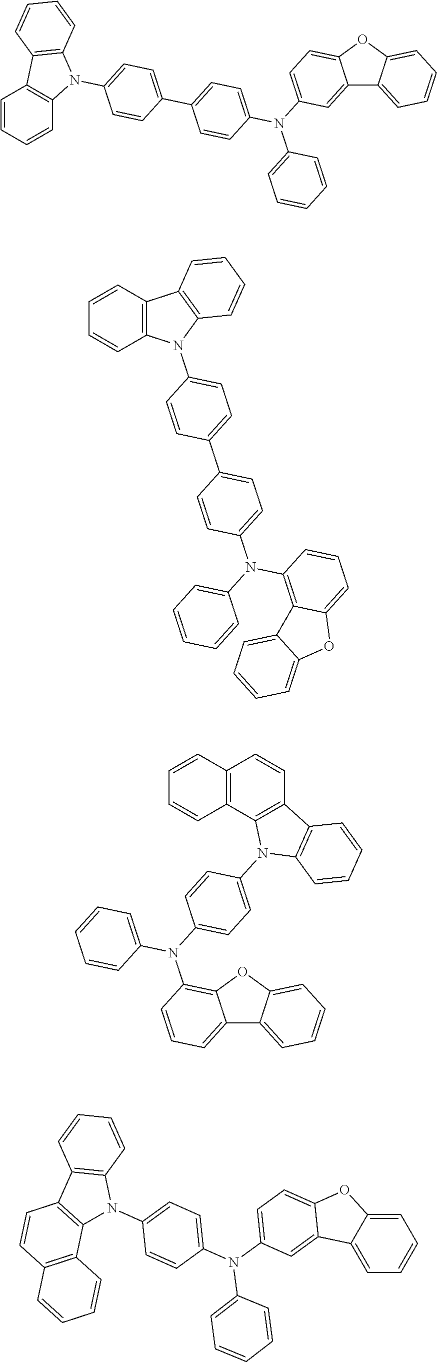 [SMILES: c1ccc(N(c2ccc(-c3ccc(-n4c5ccccc5c5ccccc54)cc3)cc2)c2ccc3oc4ccccc4c3c2)cc1 ; c1ccc(N(c2ccc(-c3ccc(-n4c5ccccc5c5ccccc54)cc3)cc2)c2cccc3oc4ccccc4c23)cc1 ; c1ccc(N(c2ccc(-n3c4ccccc4c4ccc5ccccc5c43)cc2)c2cccc3c2oc2ccccc23)cc1 ; c1ccc(N(c2ccc(-n3c4ccccc4c4ccc5ccccc5c43)cc2)c2ccc3oc4ccccc4c3c2)cc1] 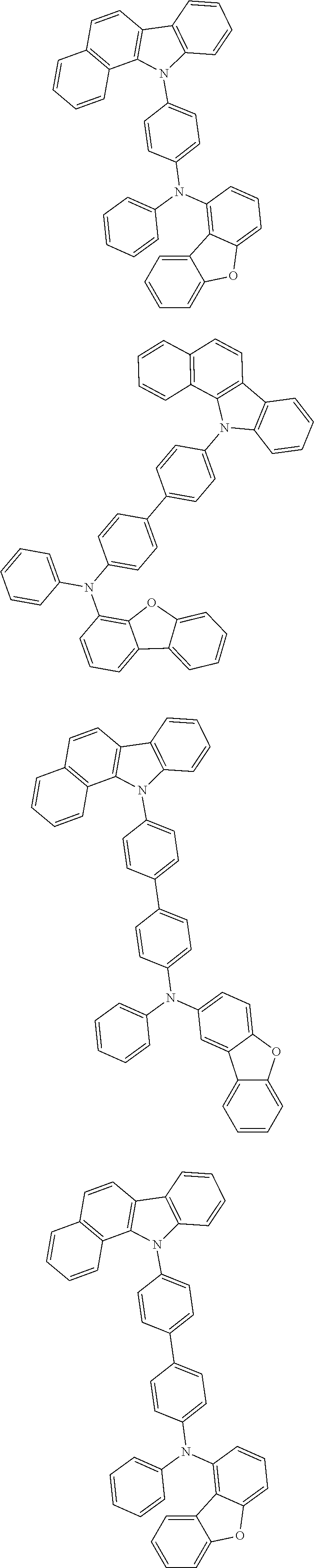 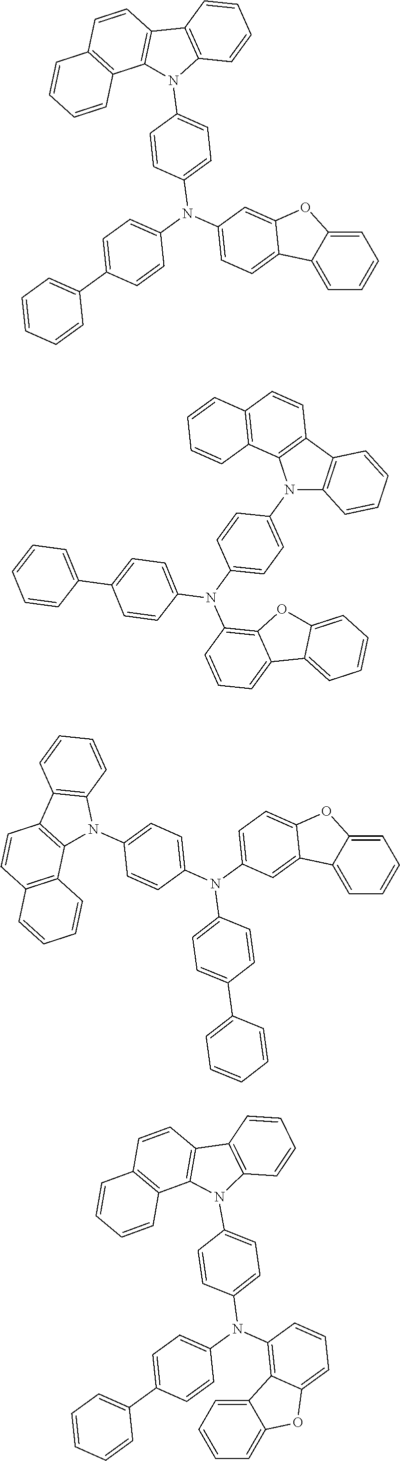 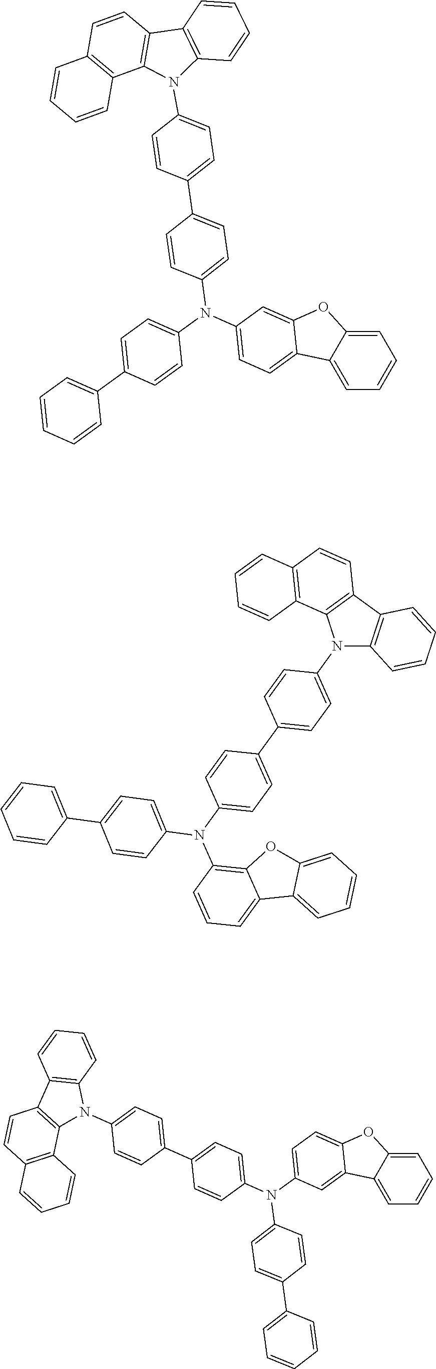 [SMILES: c1ccc(-c2ccc(N(c3ccc(-c4ccc(-n5c6ccccc6c6ccc7ccccc7c65)cc4)cc3)c3ccc4c(c3)oc3ccccc34)cc2)cc1 ; c1ccc(-c2ccc(N(c3ccc(-c4ccc(-n5c6ccccc6c6ccc7ccccc7c65)cc4)cc3)c3cccc4c3oc3ccccc34)cc2)cc1 ; c1ccc(-c2ccc(N(c3ccc(-c4ccc(-n5c6ccccc6c6ccc7ccccc7c65)cc4)cc3)c3ccc4oc5ccccc5c4c3)cc2)cc1] 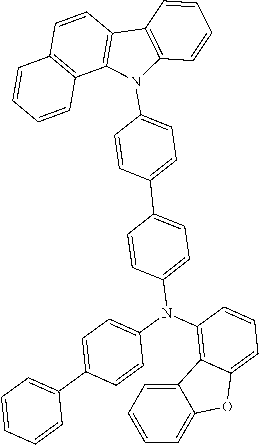 [SMILES: c1ccc(-c2ccc(N(c3ccc(-c4ccc(-n5c6ccccc6c6ccc7ccccc7c65)cc4)cc3)c3cccc4oc5ccccc5c34)cc2)cc1] 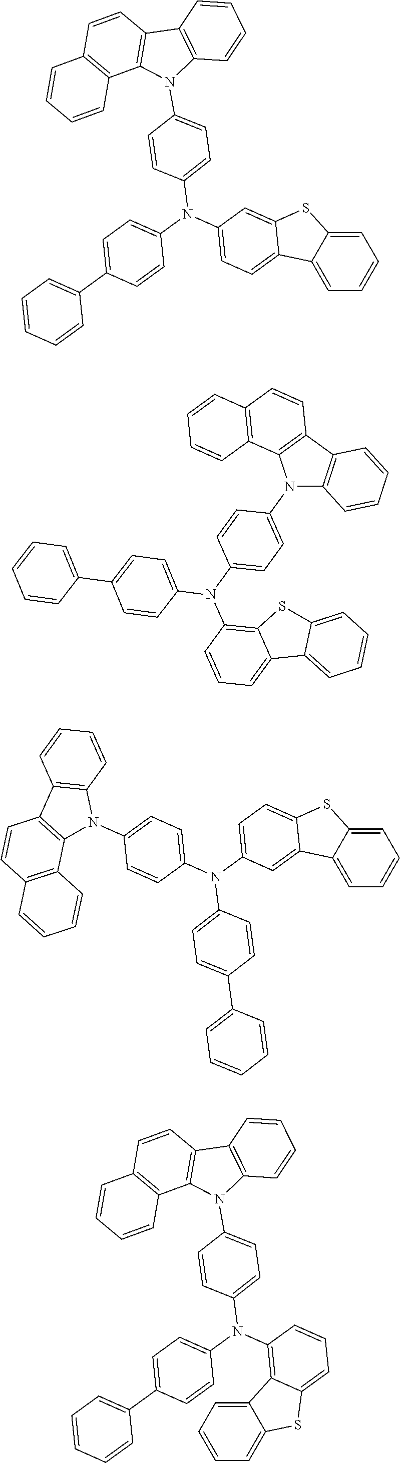 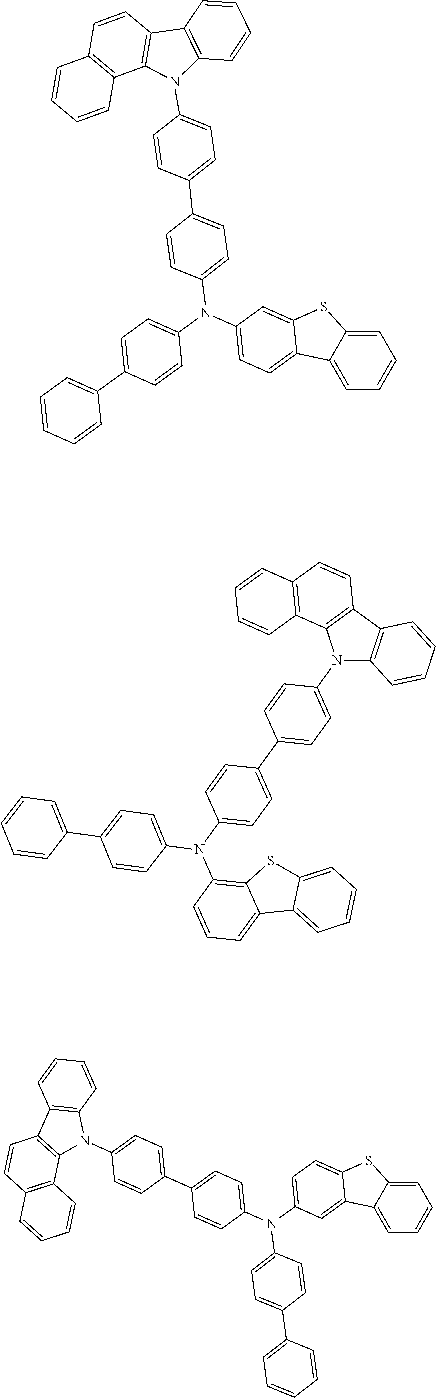 [SMILES: c1ccc(-c2ccc(N(c3ccc(-c4ccc(-n5c6ccccc6c6ccc7ccccc7c65)cc4)cc3)c3ccc4c(c3)sc3ccccc34)cc2)cc1 ; c1ccc(-c2ccc(N(c3ccc(-c4ccc(-n5c6ccccc6c6ccc7ccccc7c65)cc4)cc3)c3cccc4c3sc3ccccc34)cc2)cc1 ; c1ccc(-c2ccc(N(c3ccc(-c4ccc(-n5c6ccccc6c6ccc7ccccc7c65)cc4)cc3)c3ccc4sc5ccccc5c4c3)cc2)cc1] 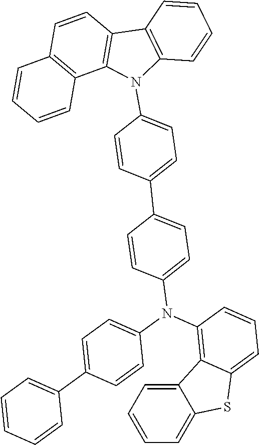 [SMILES: c1ccc(-c2ccc(N(c3ccc(-c4ccc(-n5c6ccccc6c6ccc7ccccc7c65)cc4)cc3)c3cccc4sc5ccccc5c34)cc2)cc1] 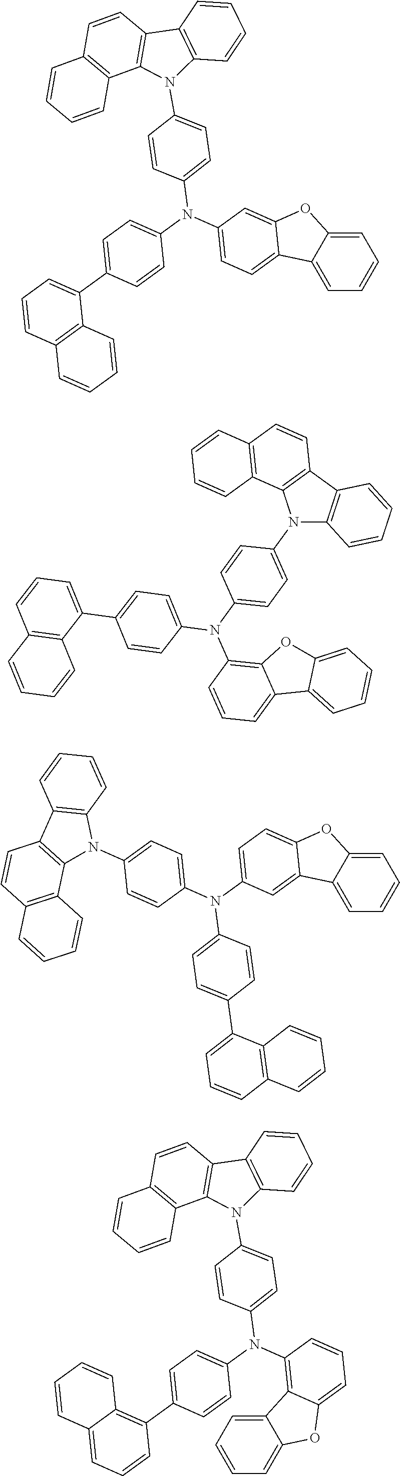 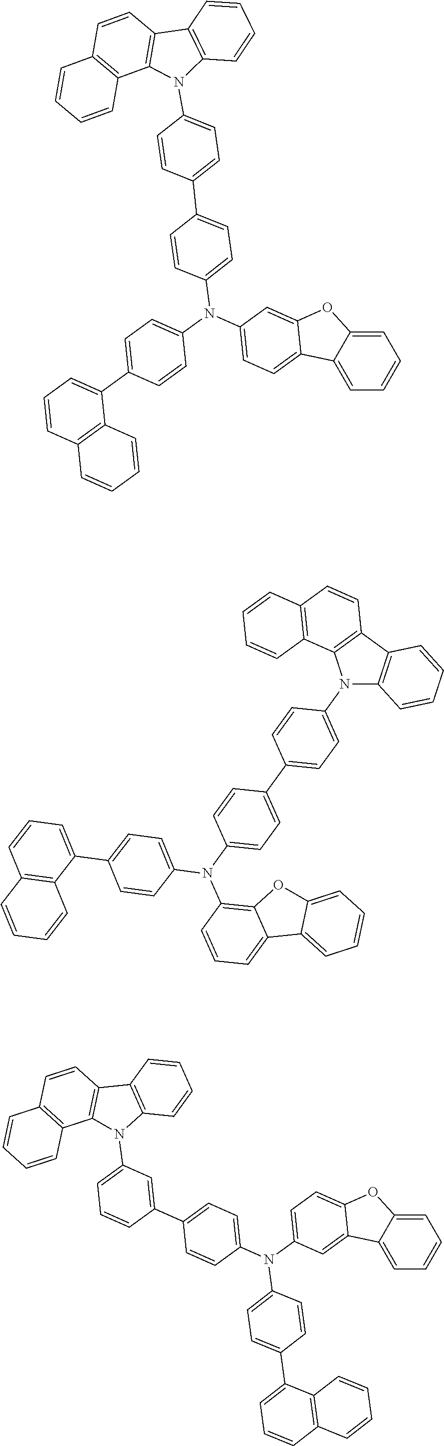 [SMILES: c1ccc2c(-c3ccc(N(c4ccc(-c5ccc(-n6c7ccccc7c7ccc8ccccc8c76)cc5)cc4)c4ccc5c(c4)oc4ccccc45)cc3)cccc2c1 ; c1ccc2c(-c3ccc(N(c4ccc(-c5ccc(-n6c7ccccc7c7ccc8ccccc8c76)cc5)cc4)c4cccc5c4oc4ccccc45)cc3)cccc2c1 ; c1cc(-c2ccc(N(c3ccc(-c4cccc5ccccc45)cc3)c3ccc4oc5ccccc5c4c3)cc2)cc(-n2c3ccccc3c3ccc4ccccc4c32)c1] 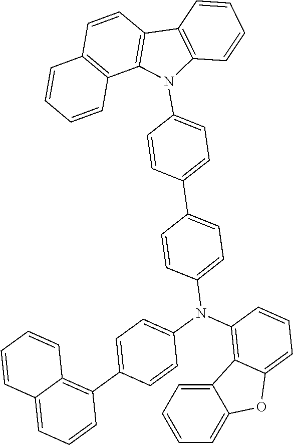 [SMILES: c1ccc2c(-c3ccc(N(c4ccc(-c5ccc(-n6c7ccccc7c7ccc8ccccc8c76)cc5)cc4)c4cccc5oc6ccccc6c45)cc3)cccc2c1] 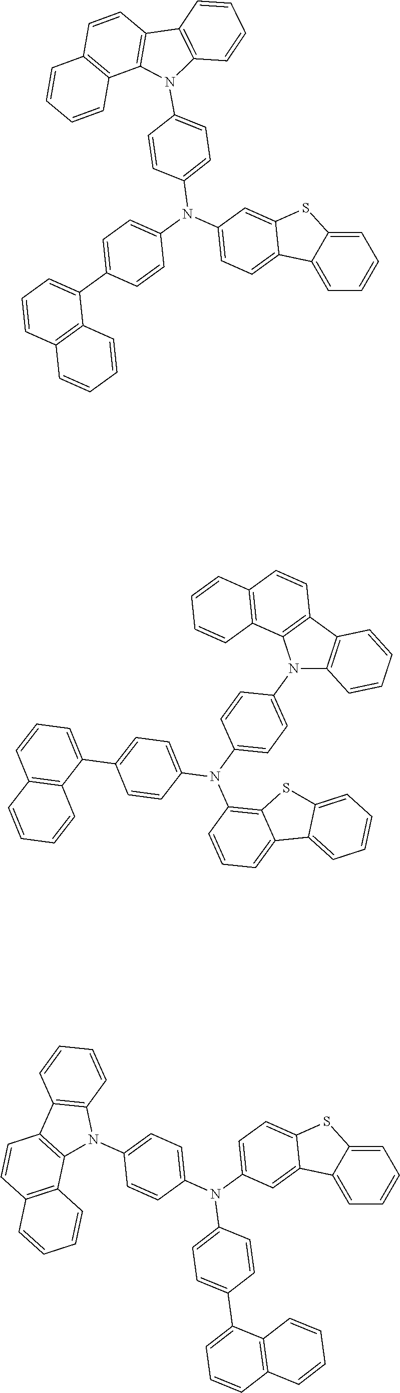 [SMILES: c1ccc2c(-c3ccc(N(c4ccc(-n5c6ccccc6c6ccc7ccccc7c65)cc4)c4ccc5c(c4)sc4ccccc45)cc3)cccc2c1 ; c1ccc2c(-c3ccc(N(c4ccc(-n5c6ccccc6c6ccc7ccccc7c65)cc4)c4cccc5c4sc4ccccc45)cc3)cccc2c1 ; c1ccc2c(-c3ccc(N(c4ccc(-n5c6ccccc6c6ccc7ccccc7c65)cc4)c4ccc5sc6ccccc6c5c4)cc3)cccc2c1] 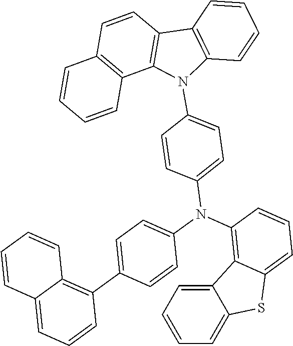 [SMILES: c1ccc2c(-c3ccc(N(c4ccc(-n5c6ccccc6c6ccc7ccccc7c65)cc4)c4cccc5sc6ccccc6c45)cc3)cccc2c1] 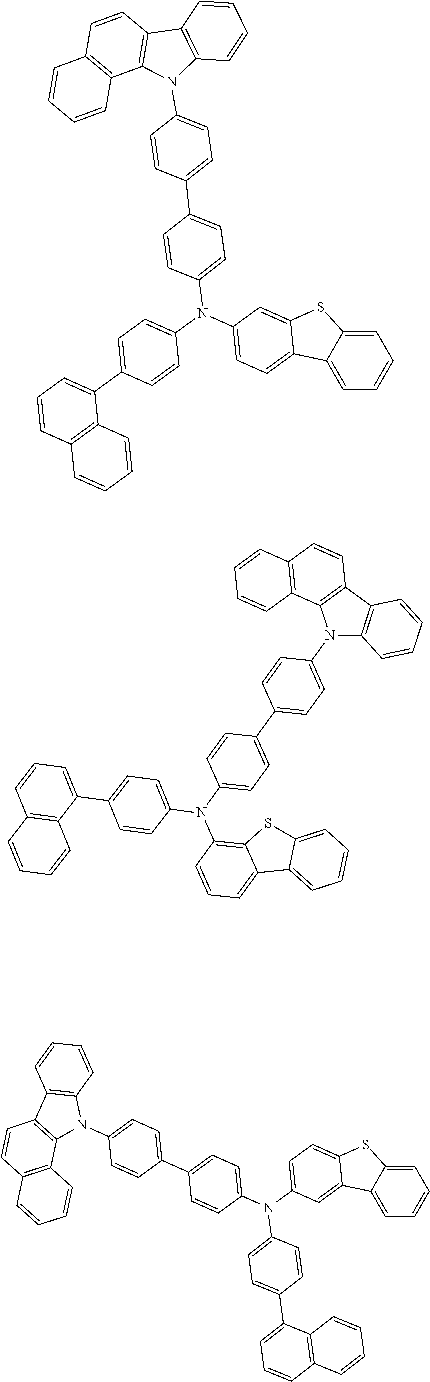 [SMILES: c1ccc2c(-c3ccc(N(c4ccc(-c5ccc(-n6c7ccccc7c7ccc8ccccc8c76)cc5)cc4)c4ccc5c(c4)sc4ccccc45)cc3)cccc2c1 ; c1ccc2c(-c3ccc(N(c4ccc(-c5ccc(-n6c7ccccc7c7ccc8ccccc8c76)cc5)cc4)c4cccc5c4sc4ccccc45)cc3)cccc2c1 ; c1ccc2c(-c3ccc(N(c4ccc(-c5ccc(-n6c7ccccc7c7ccc8ccccc8c76)cc5)cc4)c4ccc5sc6ccccc6c5c4)cc3)cccc2c1] 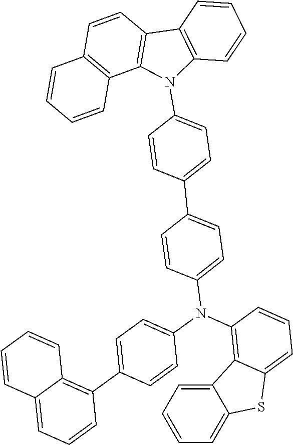 [SMILES: c1ccc2c(-c3ccc(N(c4ccc(-c5ccc(-n6c7ccccc7c7ccc8ccccc8c76)cc5)cc4)c4cccc5sc6ccccc6c45)cc3)cccc2c1] 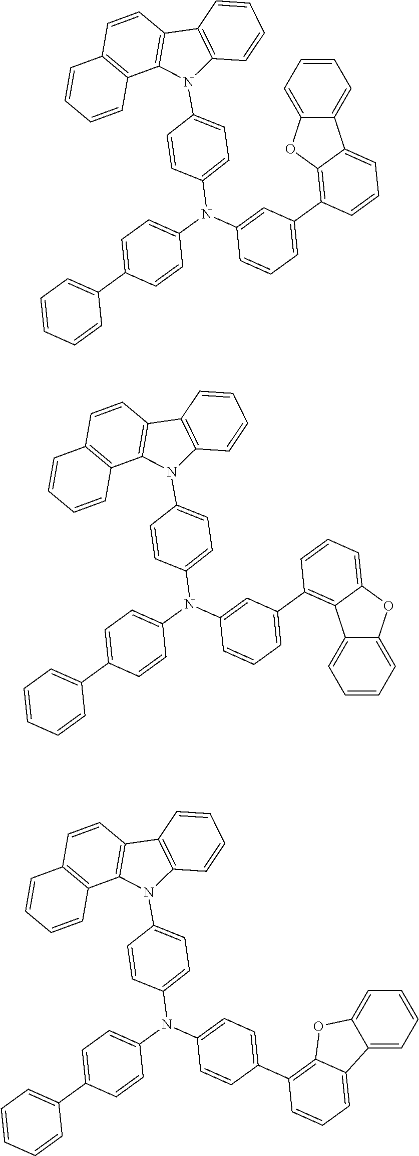 [SMILES: c1ccc(-c2ccc(N(c3ccc(-n4c5ccccc5c5ccc6ccccc6c54)cc3)c3cccc(-c4cccc5c4oc4ccccc45)c3)cc2)cc1 ; c1ccc(-c2ccc(N(c3ccc(-n4c5ccccc5c5ccc6ccccc6c54)cc3)c3cccc(-c4cccc5oc6ccccc6c45)c3)cc2)cc1 ; c1ccc(-c2ccc(N(c3ccc(-c4cccc5c4oc4ccccc45)cc3)c3ccc(-n4c5ccccc5c5ccc6ccccc6c54)cc3)cc2)cc1] 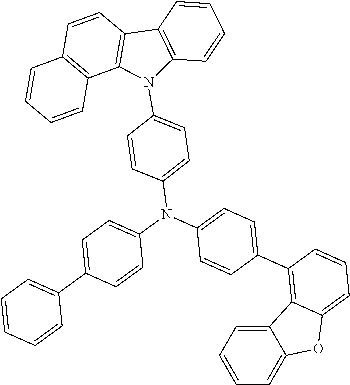 [SMILES: c1ccc(-c2ccc(N(c3ccc(-c4cccc5oc6ccccc6c45)cc3)c3ccc(-n4c5ccccc5c5ccc6ccccc6c54)cc3)cc2)cc1] 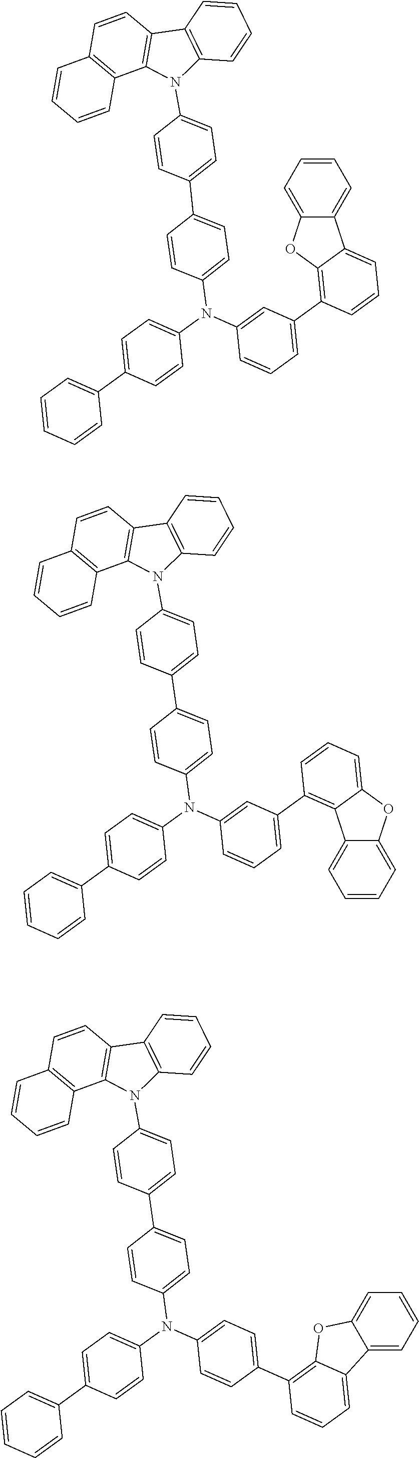 [SMILES: c1ccc(-c2ccc(N(c3ccc(-c4ccc(-n5c6ccccc6c6ccc7ccccc7c65)cc4)cc3)c3cccc(-c4cccc5c4oc4ccccc45)c3)cc2)cc1 ; c1ccc(-c2ccc(N(c3ccc(-c4ccc(-n5c6ccccc6c6ccc7ccccc7c65)cc4)cc3)c3cccc(-c4cccc5oc6ccccc6c45)c3)cc2)cc1 ; c1ccc(-c2ccc(N(c3ccc(-c4ccc(-n5c6ccccc6c6ccc7ccccc7c65)cc4)cc3)c3ccc(-c4cccc5c4oc4ccccc45)cc3)cc2)cc1] 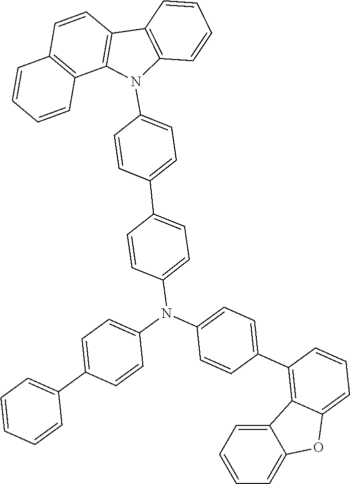 [SMILES: c1ccc(-c2ccc(N(c3ccc(-c4ccc(-n5c6ccccc6c6ccc7ccccc7c65)cc4)cc3)c3ccc(-c4cccc5oc6ccccc6c45)cc3)cc2)cc1] 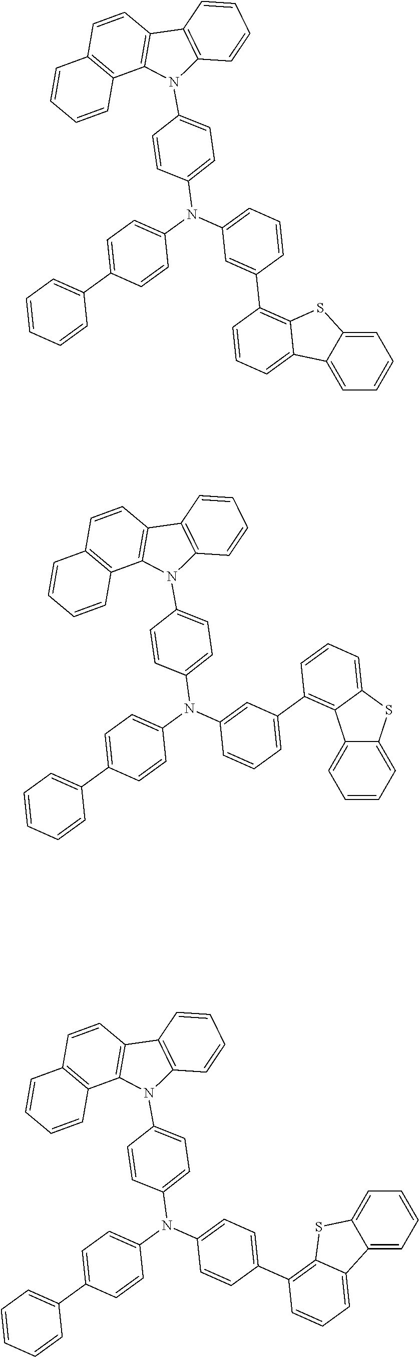 [SMILES: c1ccc(-c2ccc(N(c3ccc(-n4c5ccccc5c5ccc6ccccc6c54)cc3)c3cccc(-c4cccc5c4sc4ccccc45)c3)cc2)cc1 ; c1ccc(-c2ccc(N(c3ccc(-n4c5ccccc5c5ccc6ccccc6c54)cc3)c3cccc(-c4cccc5sc6ccccc6c45)c3)cc2)cc1 ; c1ccc(-c2ccc(N(c3ccc(-c4cccc5c4sc4ccccc45)cc3)c3ccc(-n4c5ccccc5c5ccc6ccccc6c54)cc3)cc2)cc1] 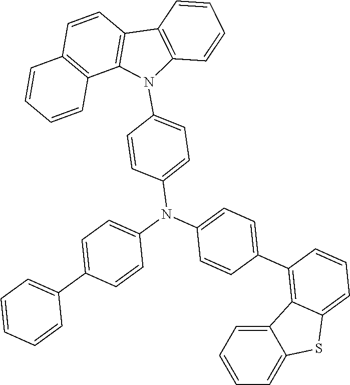 [SMILES: c1ccc(-c2ccc(N(c3ccc(-c4cccc5sc6ccccc6c45)cc3)c3ccc(-n4c5ccccc5c5ccc6ccccc6c54)cc3)cc2)cc1] 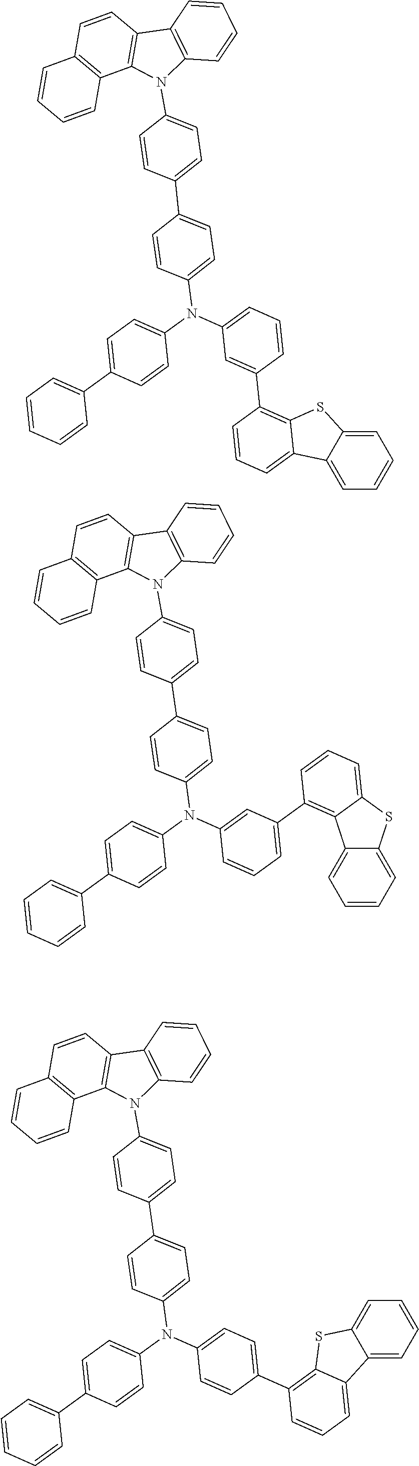 [SMILES: c1ccc(-c2ccc(N(c3ccc(-c4ccc(-n5c6ccccc6c6ccc7ccccc7c65)cc4)cc3)c3cccc(-c4cccc5c4sc4ccccc45)c3)cc2)cc1 ; c1ccc(-c2ccc(N(c3ccc(-c4ccc(-n5c6ccccc6c6ccc7ccccc7c65)cc4)cc3)c3cccc(-c4cccc5sc6ccccc6c45)c3)cc2)cc1 ; c1ccc(-c2ccc(N(c3ccc(-c4ccc(-n5c6ccccc6c6ccc7ccccc7c65)cc4)cc3)c3ccc(-c4cccc5c4sc4ccccc45)cc3)cc2)cc1] 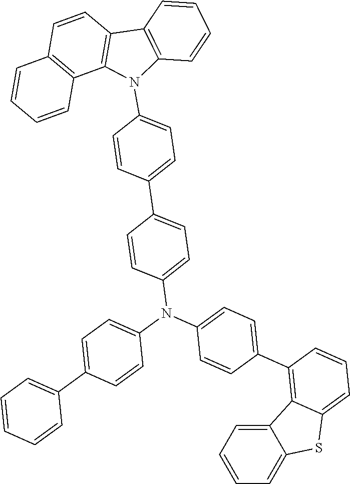 [SMILES: c1ccc(-c2ccc(N(c3ccc(-c4ccc(-n5c6ccccc6c6ccc7ccccc7c65)cc4)cc3)c3ccc(-c4cccc5sc6ccccc6c45)cc3)cc2)cc1] 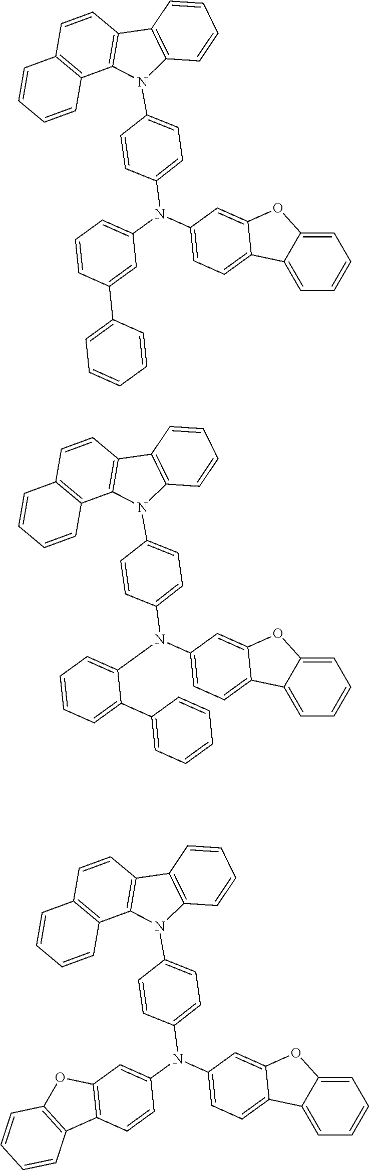 [SMILES: c1ccc(-c2cccc(N(c3ccc(-n4c5ccccc5c5ccc6ccccc6c54)cc3)c3ccc4c(c3)oc3ccccc34)c2)cc1 ; c1ccc(-c2ccccc2N(c2ccc(-n3c4ccccc4c4ccc5ccccc5c43)cc2)c2ccc3c(c2)oc2ccccc23)cc1 ; c1ccc2c(c1)ccc1c3ccccc3n(-c3ccc(N(c4ccc5c(c4)oc4ccccc45)c4ccc5c(c4)oc4ccccc45)cc3)c21] 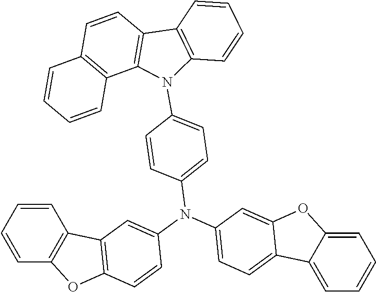 [SMILES: c1ccc2c(c1)ccc1c3ccccc3n(-c3ccc(N(c4ccc5c(c4)oc4ccccc45)c4ccc5oc6ccccc6c5c4)cc3)c21] 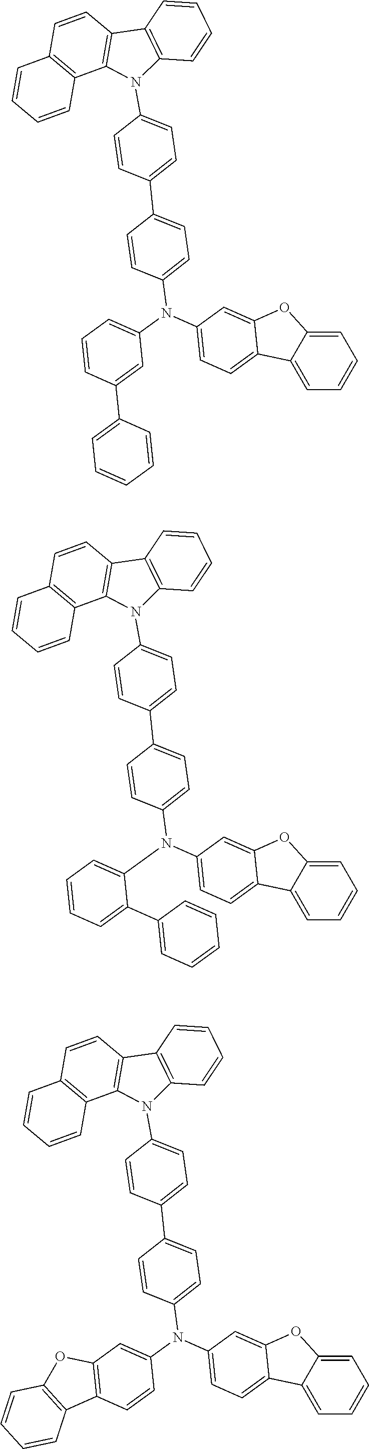 [SMILES: c1ccc(-c2cccc(N(c3ccc(-c4ccc(-n5c6ccccc6c6ccc7ccccc7c65)cc4)cc3)c3ccc4c(c3)oc3ccccc34)c2)cc1 ; c1ccc(-c2ccccc2N(c2ccc(-c3ccc(-n4c5ccccc5c5ccc6ccccc6c54)cc3)cc2)c2ccc3c(c2)oc2ccccc23)cc1 ; c1ccc2c(c1)ccc1c3ccccc3n(-c3ccc(-c4ccc(N(c5ccc6c(c5)oc5ccccc56)c5ccc6c(c5)oc5ccccc56)cc4)cc3)c21] 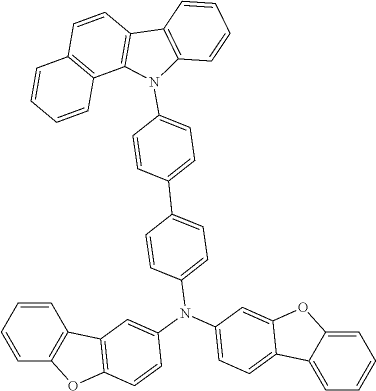 [SMILES: c1ccc2c(c1)ccc1c3ccccc3n(-c3ccc(-c4ccc(N(c5ccc6c(c5)oc5ccccc56)c5ccc6oc7ccccc7c6c5)cc4)cc3)c21] 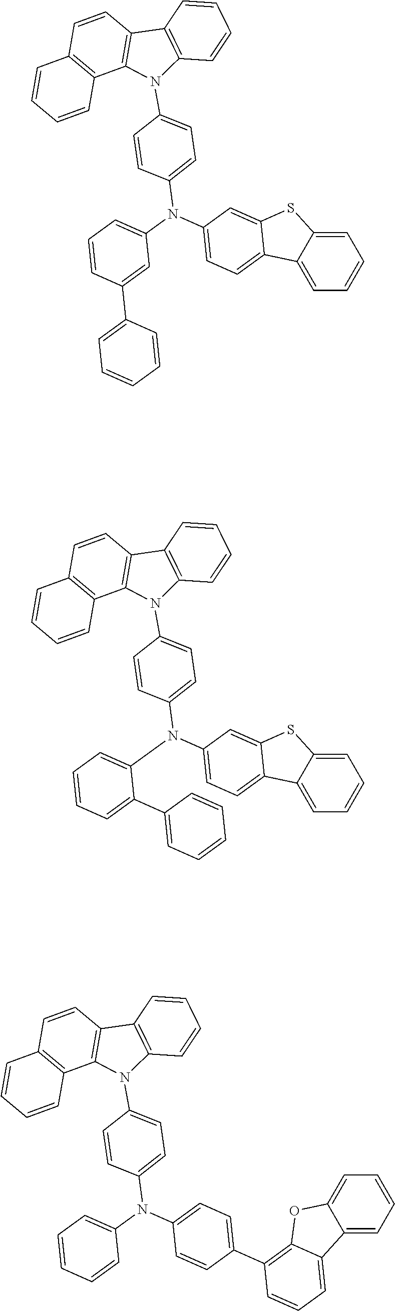 [SMILES: c1ccc(-c2cccc(N(c3ccc(-n4c5ccccc5c5ccc6ccccc6c54)cc3)c3ccc4c(c3)sc3ccccc34)c2)cc1 ; c1ccc(-c2ccccc2N(c2ccc(-n3c4ccccc4c4ccc5ccccc5c43)cc2)c2ccc3c(c2)sc2ccccc23)cc1 ; c1ccc(N(c2ccc(-c3cccc4c3oc3ccccc34)cc2)c2ccc(-n3c4ccccc4c4ccc5ccccc5c43)cc2)cc1] 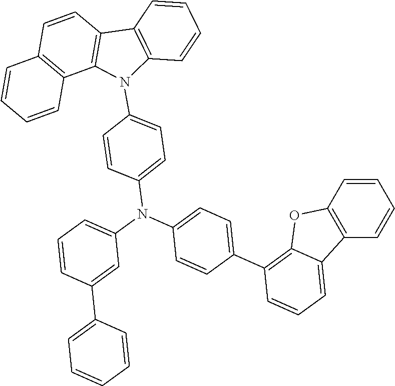 [SMILES: c1ccc(-c2cccc(N(c3ccc(-c4cccc5c4oc4ccccc45)cc3)c3ccc(-n4c5ccccc5c5ccc6ccccc6c54)cc3)c2)cc1] 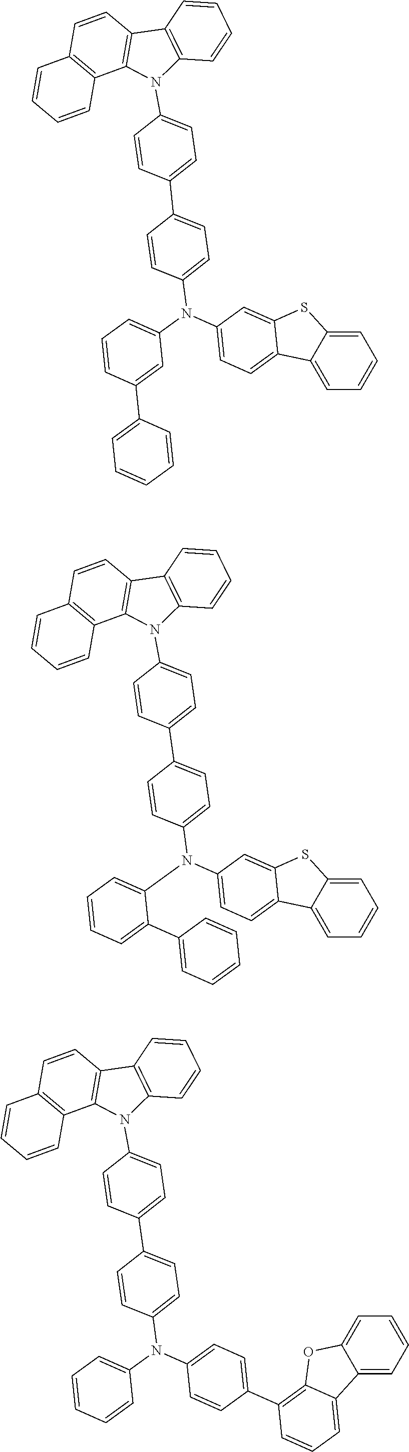 [SMILES: c1ccc(-c2cccc(N(c3ccc(-c4ccc(-n5c6ccccc6c6ccc7ccccc7c65)cc4)cc3)c3ccc4c(c3)sc3ccccc34)c2)cc1 ; c1ccc(-c2ccccc2N(c2ccc(-c3ccc(-n4c5ccccc5c5ccc6ccccc6c54)cc3)cc2)c2ccc3c(c2)sc2ccccc23)cc1 ; c1ccc(N(c2ccc(-c3ccc(-n4c5ccccc5c5ccc6ccccc6c54)cc3)cc2)c2ccc(-c3cccc4c3oc3ccccc34)cc2)cc1] 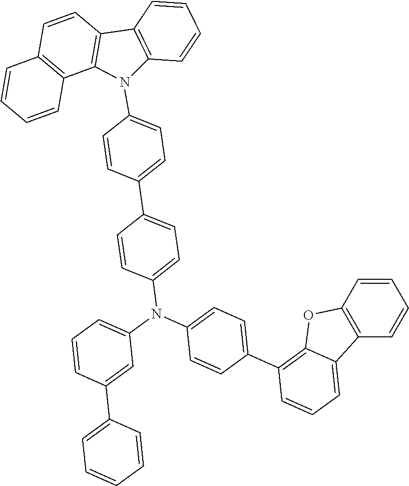 [SMILES: c1ccc(-c2cccc(N(c3ccc(-c4ccc(-n5c6ccccc6c6ccc7ccccc7c65)cc4)cc3)c3ccc(-c4cccc5c4oc4ccccc45)cc3)c2)cc1] 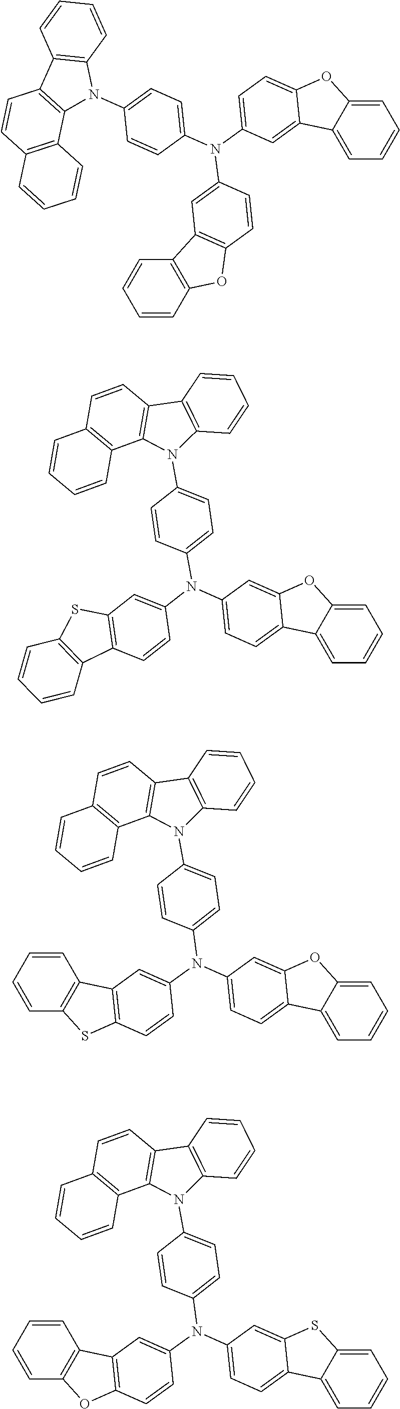 [SMILES: c1ccc2c(c1)ccc1c3ccccc3n(-c3ccc(N(c4ccc5oc6ccccc6c5c4)c4ccc5oc6ccccc6c5c4)cc3)c21 ; c1ccc2c(c1)ccc1c3ccccc3n(-c3ccc(N(c4ccc5c(c4)oc4ccccc45)c4ccc5c(c4)sc4ccccc45)cc3)c21 ; c1ccc2c(c1)ccc1c3ccccc3n(-c3ccc(N(c4ccc5c(c4)oc4ccccc45)c4ccc5sc6ccccc6c5c4)cc3)c21 ; c1ccc2c(c1)ccc1c3ccccc3n(-c3ccc(N(c4ccc5c(c4)sc4ccccc45)c4ccc5oc6ccccc6c5c4)cc3)c21] 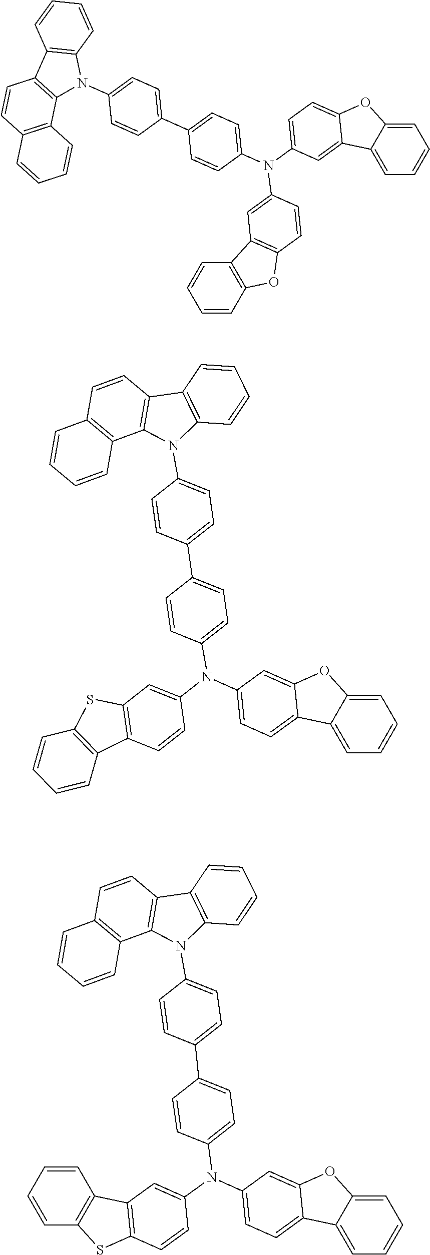 [SMILES: c1ccc2c(c1)ccc1c3ccccc3n(-c3ccc(-c4ccc(N(c5ccc6oc7ccccc7c6c5)c5ccc6oc7ccccc7c6c5)cc4)cc3)c21 ; c1ccc2c(c1)ccc1c3ccccc3n(-c3ccc(-c4ccc(N(c5ccc6c(c5)oc5ccccc56)c5ccc6c(c5)sc5ccccc56)cc4)cc3)c21 ; c1ccc2c(c1)ccc1c3ccccc3n(-c3ccc(-c4ccc(N(c5ccc6c(c5)oc5ccccc56)c5ccc6sc7ccccc7c6c5)cc4)cc3)c21] 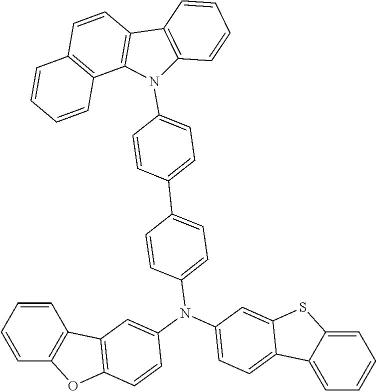 [SMILES: c1ccc2c(c1)ccc1c3ccccc3n(-c3ccc(-c4ccc(N(c5ccc6c(c5)sc5ccccc56)c5ccc6oc7ccccc7c6c5)cc4)cc3)c21] 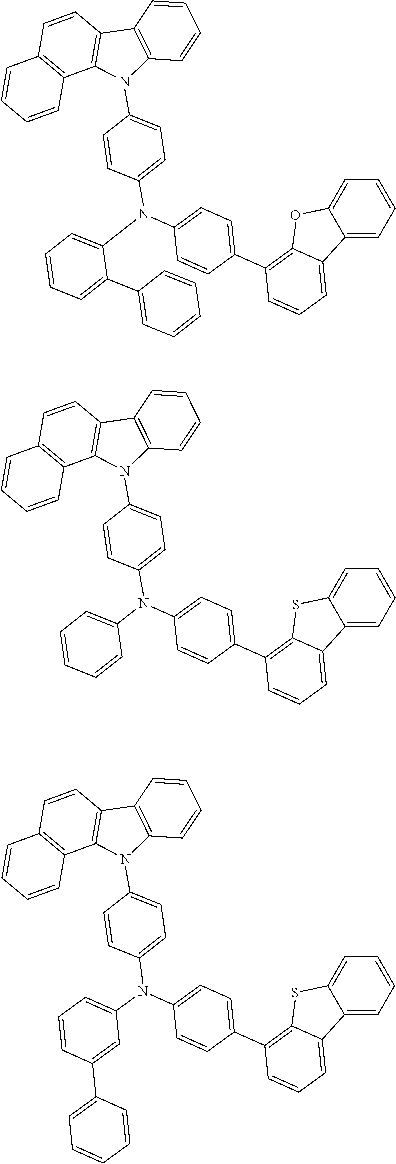 [SMILES: c1ccc(-c2ccccc2N(c2ccc(-c3cccc4c3oc3ccccc34)cc2)c2ccc(-n3c4ccccc4c4ccc5ccccc5c43)cc2)cc1 ; c1ccc(N(c2ccc(-c3cccc4c3sc3ccccc34)cc2)c2ccc(-n3c4ccccc4c4ccc5ccccc5c43)cc2)cc1 ; c1ccc(-c2cccc(N(c3ccc(-c4cccc5c4sc4ccccc45)cc3)c3ccc(-n4c5ccccc5c5ccc6ccccc6c54)cc3)c2)cc1] 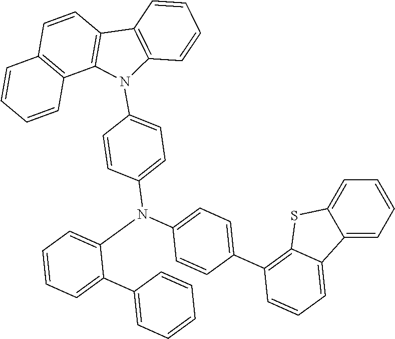 [SMILES: c1ccc(-c2ccccc2N(c2ccc(-c3cccc4c3sc3ccccc34)cc2)c2ccc(-n3c4ccccc4c4ccc5ccccc5c43)cc2)cc1] 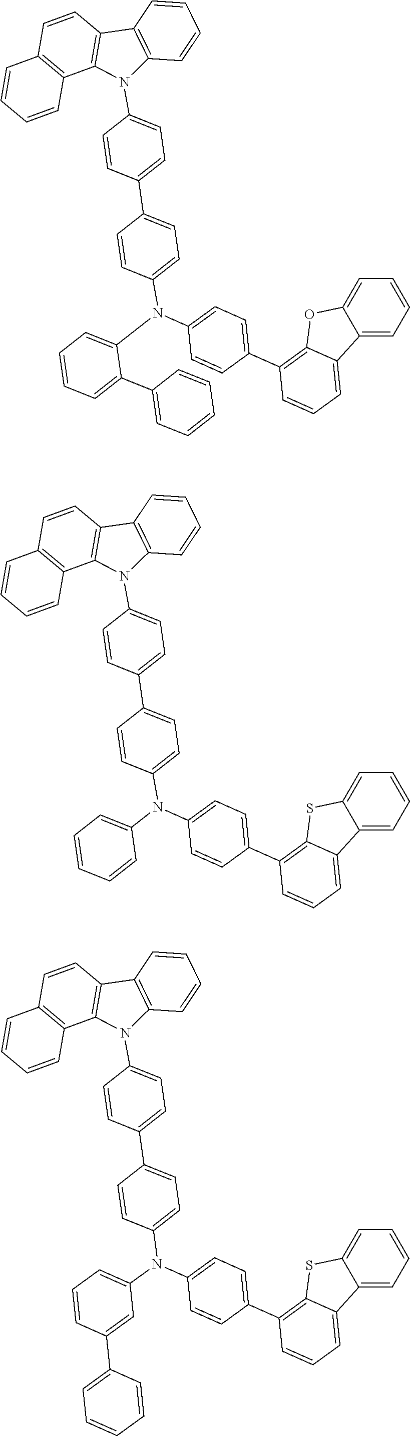 [SMILES: c1ccc(-c2ccccc2N(c2ccc(-c3ccc(-n4c5ccccc5c5ccc6ccccc6c54)cc3)cc2)c2ccc(-c3cccc4c3oc3ccccc34)cc2)cc1 ; c1ccc(N(c2ccc(-c3ccc(-n4c5ccccc5c5ccc6ccccc6c54)cc3)cc2)c2ccc(-c3cccc4c3sc3ccccc34)cc2)cc1 ; c1ccc(-c2cccc(N(c3ccc(-c4ccc(-n5c6ccccc6c6ccc7ccccc7c65)cc4)cc3)c3ccc(-c4cccc5c4sc4ccccc45)cc3)c2)cc1] 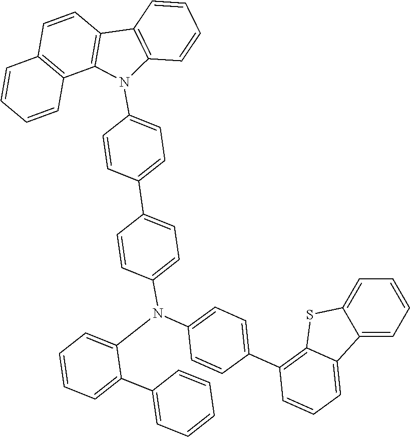 [SMILES: c1ccc(-c2ccccc2N(c2ccc(-c3ccc(-n4c5ccccc5c5ccc6ccccc6c54)cc3)cc2)c2ccc(-c3cccc4c3sc3ccccc34)cc2)cc1] 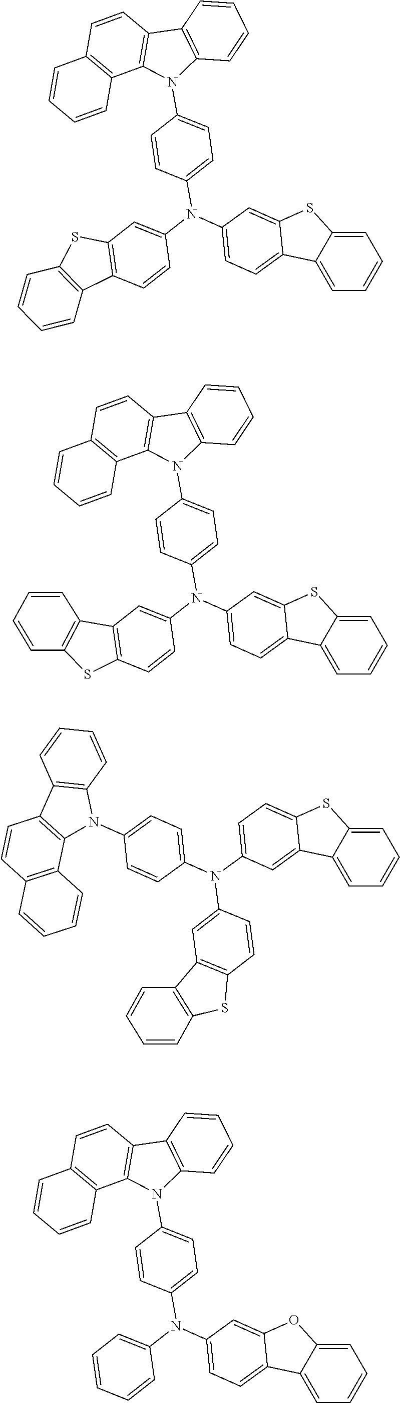 [SMILES: c1ccc2c(c1)ccc1c3ccccc3n(-c3ccc(N(c4ccc5c(c4)sc4ccccc45)c4ccc5c(c4)sc4ccccc45)cc3)c21 ; c1ccc2c(c1)ccc1c3ccccc3n(-c3ccc(N(c4ccc5c(c4)sc4ccccc45)c4ccc5sc6ccccc6c5c4)cc3)c21 ; c1ccc2c(c1)ccc1c3ccccc3n(-c3ccc(N(c4ccc5sc6ccccc6c5c4)c4ccc5sc6ccccc6c5c4)cc3)c21 ; c1ccc(N(c2ccc(-n3c4ccccc4c4ccc5ccccc5c43)cc2)c2ccc3c(c2)oc2ccccc23)cc1] 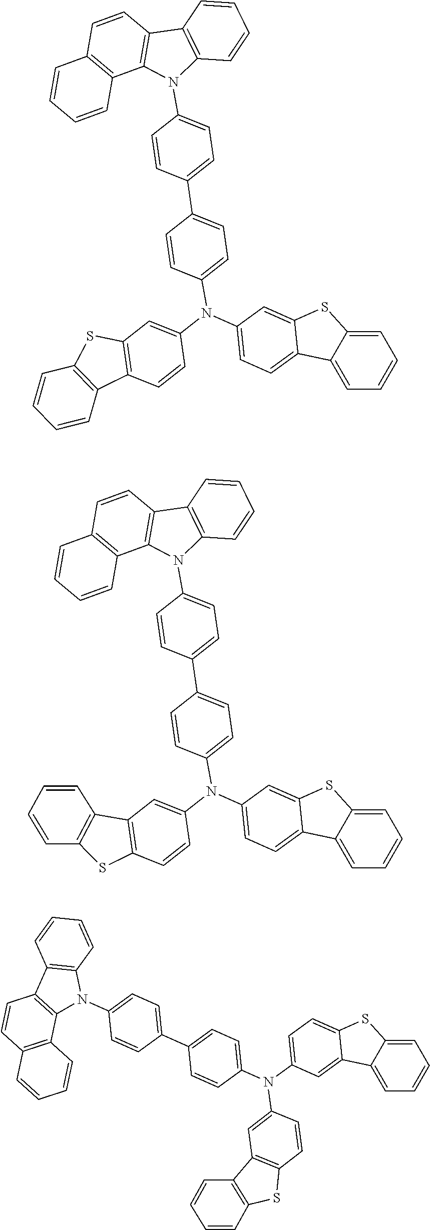 [SMILES: c1ccc2c(c1)ccc1c3ccccc3n(-c3ccc(-c4ccc(N(c5ccc6c(c5)sc5ccccc56)c5ccc6c(c5)sc5ccccc56)cc4)cc3)c21 ; c1ccc2c(c1)ccc1c3ccccc3n(-c3ccc(-c4ccc(N(c5ccc6c(c5)sc5ccccc56)c5ccc6sc7ccccc7c6c5)cc4)cc3)c21 ; c1ccc2c(c1)ccc1c3ccccc3n(-c3ccc(-c4ccc(N(c5ccc6sc7ccccc7c6c5)c5ccc6sc7ccccc7c6c5)cc4)cc3)c21] 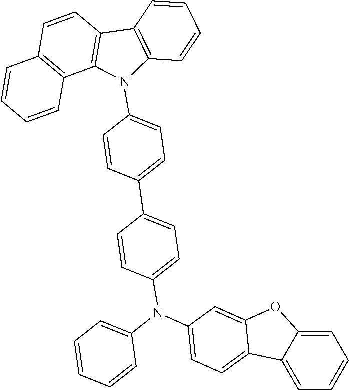 [SMILES: c1ccc(N(c2ccc(-c3ccc(-n4c5ccccc5c5ccc6ccccc6c54)cc3)cc2)c2ccc3c(c2)oc2ccccc23)cc1] 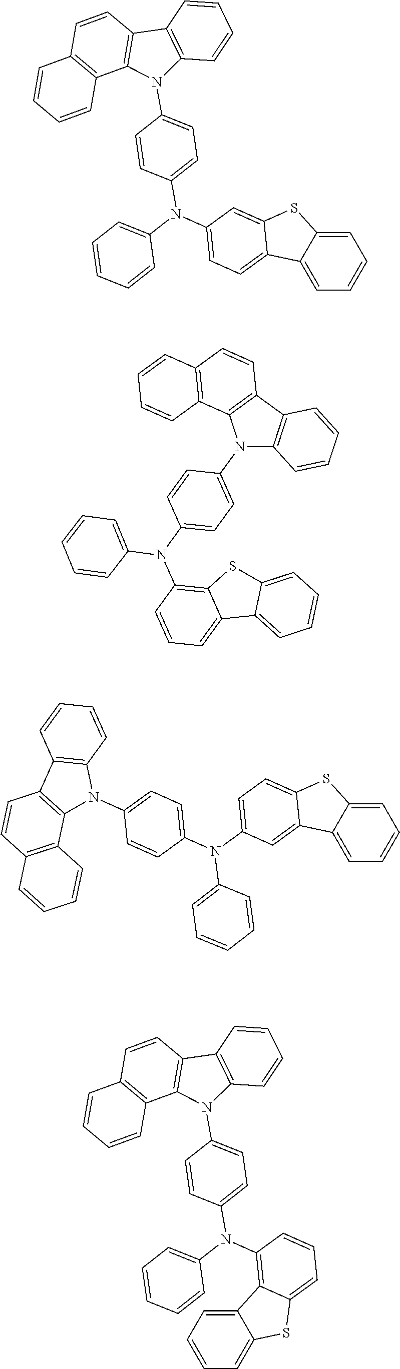 [SMILES: c1ccc(N(c2ccc(-n3c4ccccc4c4ccc5ccccc5c43)cc2)c2ccc3c(c2)sc2ccccc23)cc1 ; c1ccc(N(c2ccc(-n3c4ccccc4c4ccc5ccccc5c43)cc2)c2cccc3c2sc2ccccc23)cc1 ; c1ccc(N(c2ccc(-n3c4ccccc4c4ccc5ccccc5c43)cc2)c2ccc3sc4ccccc4c3c2)cc1 ; c1ccc(N(c2ccc(-n3c4ccccc4c4ccc5ccccc5c43)cc2)c2cccc3sc4ccccc4c23)cc1] 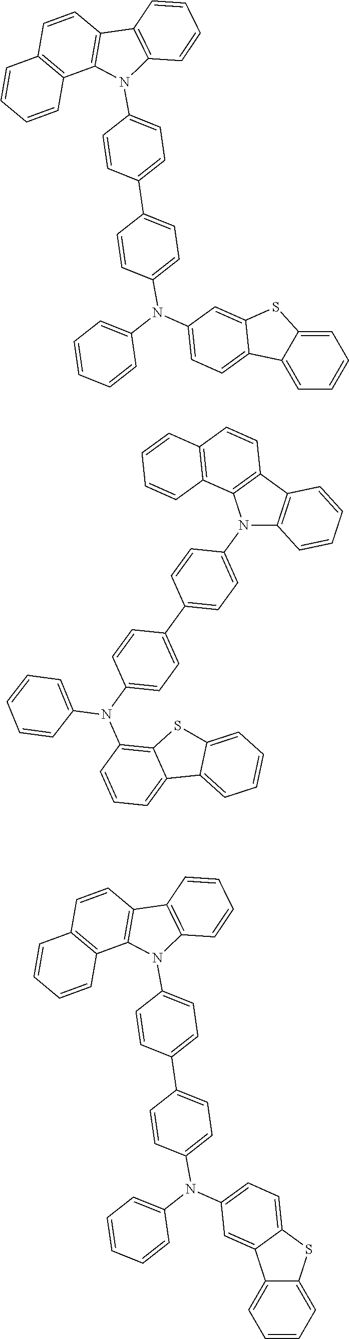 [SMILES: c1ccc(N(c2ccc(-c3ccc(-n4c5ccccc5c5ccc6ccccc6c54)cc3)cc2)c2ccc3c(c2)sc2ccccc23)cc1 ; c1ccc(N(c2ccc(-c3ccc(-n4c5ccccc5c5ccc6ccccc6c54)cc3)cc2)c2cccc3c2sc2ccccc23)cc1 ; c1ccc(N(c2ccc(-c3ccc(-n4c5ccccc5c5ccc6ccccc6c54)cc3)cc2)c2ccc3sc4ccccc4c3c2)cc1] 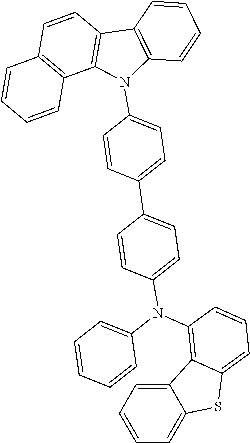 [SMILES: c1ccc(N(c2ccc(-c3ccc(-n4c5ccccc5c5ccc6ccccc6c54)cc3)cc2)c2cccc3sc4ccccc4c23)cc1] 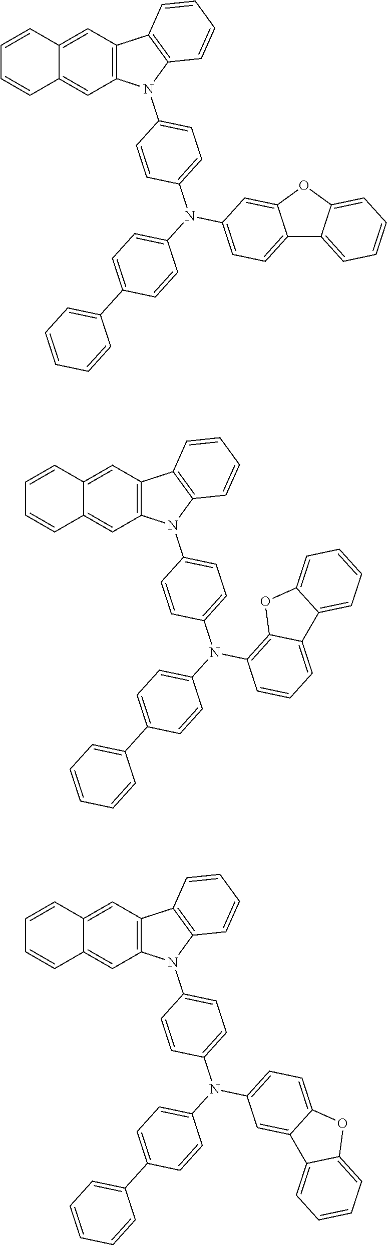 [SMILES: c1ccc(-c2ccc(N(c3ccc(-n4c5ccccc5c5cc6ccccc6cc54)cc3)c3ccc4c(c3)oc3ccccc34)cc2)cc1 ; c1ccc(-c2ccc(N(c3ccc(-n4c5ccccc5c5cc6ccccc6cc54)cc3)c3cccc4c3oc3ccccc34)cc2)cc1 ; c1ccc(-c2ccc(N(c3ccc(-n4c5ccccc5c5cc6ccccc6cc54)cc3)c3ccc4oc5ccccc5c4c3)cc2)cc1] 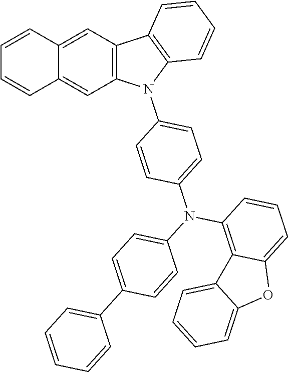 [SMILES: c1ccc(-c2ccc(N(c3ccc(-n4c5ccccc5c5cc6ccccc6cc54)cc3)c3cccc4oc5ccccc5c34)cc2)cc1] 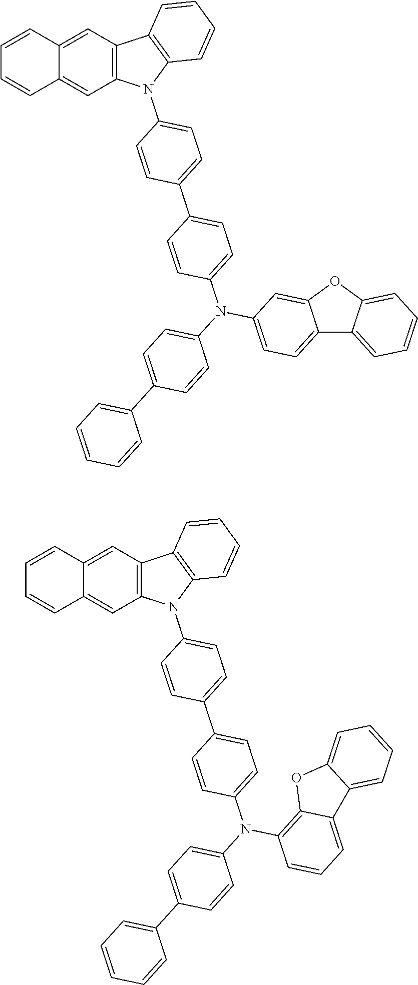 [SMILES: c1ccc(-c2ccc(N(c3ccc(-c4ccc(-n5c6ccccc6c6cc7ccccc7cc65)cc4)cc3)c3ccc4c(c3)oc3ccccc34)cc2)cc1 ; c1ccc(-c2ccc(N(c3ccc(-c4ccc(-n5c6ccccc6c6cc7ccccc7cc65)cc4)cc3)c3cccc4c3oc3ccccc34)cc2)cc1] 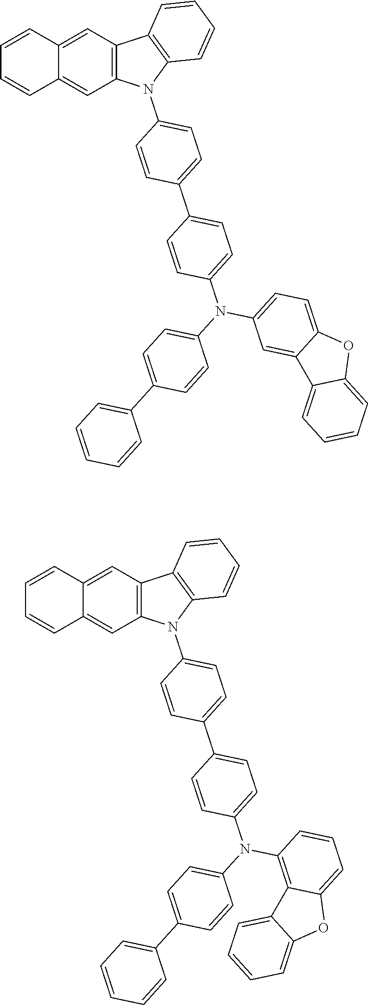 [SMILES: c1ccc(-c2ccc(N(c3ccc(-c4ccc(-n5c6ccccc6c6cc7ccccc7cc65)cc4)cc3)c3ccc4oc5ccccc5c4c3)cc2)cc1 ; c1ccc(-c2ccc(N(c3ccc(-c4ccc(-n5c6ccccc6c6cc7ccccc7cc65)cc4)cc3)c3cccc4oc5ccccc5c34)cc2)cc1] 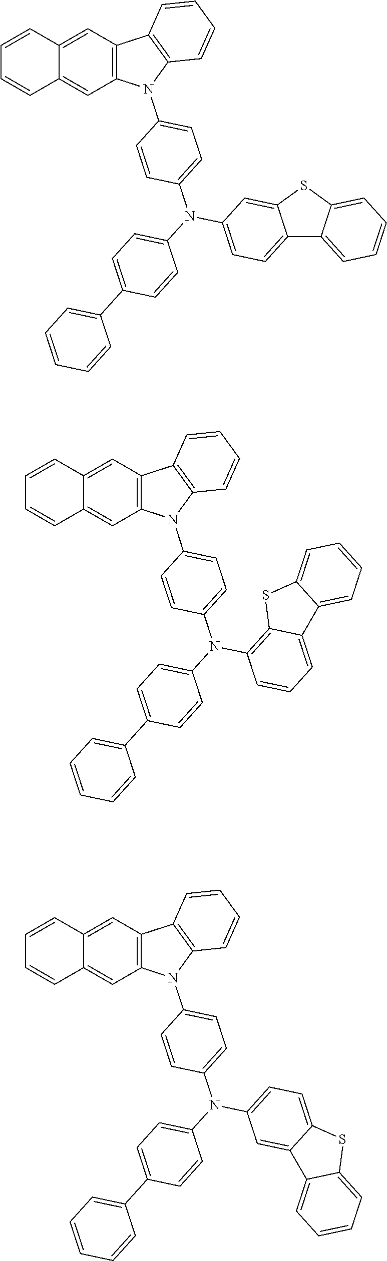 [SMILES: c1ccc(-c2ccc(N(c3ccc(-n4c5ccccc5c5cc6ccccc6cc54)cc3)c3ccc4c(c3)sc3ccccc34)cc2)cc1 ; c1ccc(-c2ccc(N(c3ccc(-n4c5ccccc5c5cc6ccccc6cc54)cc3)c3cccc4c3sc3ccccc34)cc2)cc1 ; c1ccc(-c2ccc(N(c3ccc(-n4c5ccccc5c5cc6ccccc6cc54)cc3)c3ccc4sc5ccccc5c4c3)cc2)cc1] 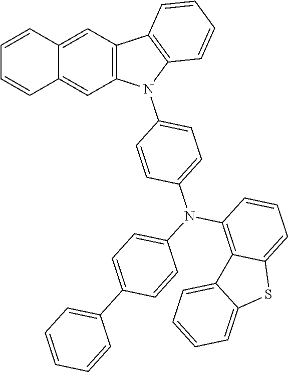 [SMILES: c1ccc(-c2ccc(N(c3ccc(-n4c5ccccc5c5cc6ccccc6cc54)cc3)c3cccc4sc5ccccc5c34)cc2)cc1] 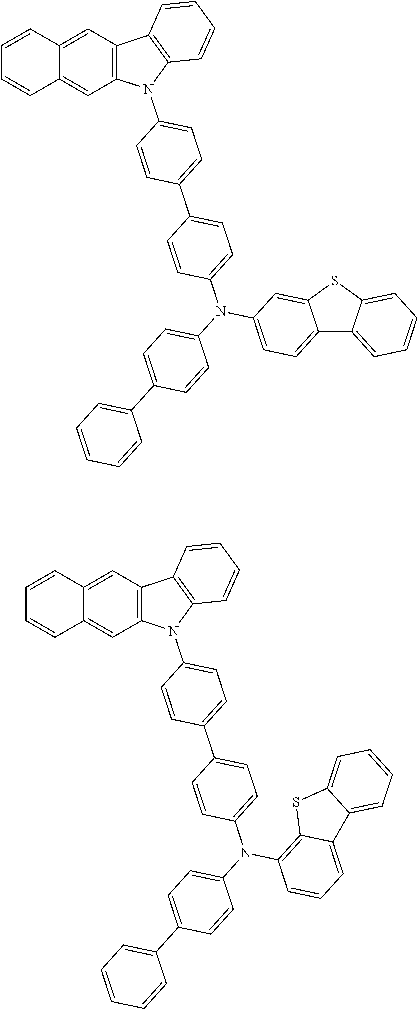 [SMILES: c1ccc(-c2ccc(N(c3ccc(-c4ccc(-n5c6ccccc6c6cc7ccccc7cc65)cc4)cc3)c3ccc4c(c3)sc3ccccc34)cc2)cc1 ; c1ccc(-c2ccc(N(c3ccc(-c4ccc(-n5c6ccccc6c6cc7ccccc7cc65)cc4)cc3)c3cccc4c3sc3ccccc34)cc2)cc1] 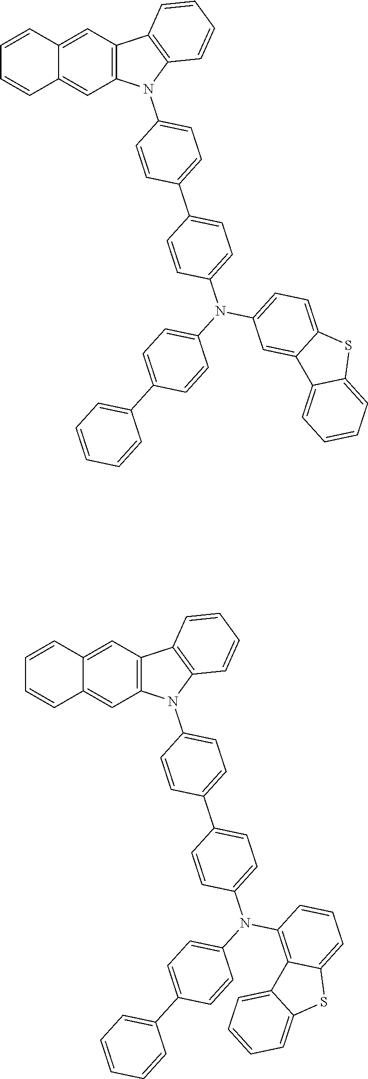 [SMILES: c1ccc(-c2ccc(N(c3ccc(-c4ccc(-n5c6ccccc6c6cc7ccccc7cc65)cc4)cc3)c3ccc4sc5ccccc5c4c3)cc2)cc1 ; c1ccc(-c2ccc(N(c3ccc(-c4ccc(-n5c6ccccc6c6cc7ccccc7cc65)cc4)cc3)c3cccc4sc5ccccc5c34)cc2)cc1] 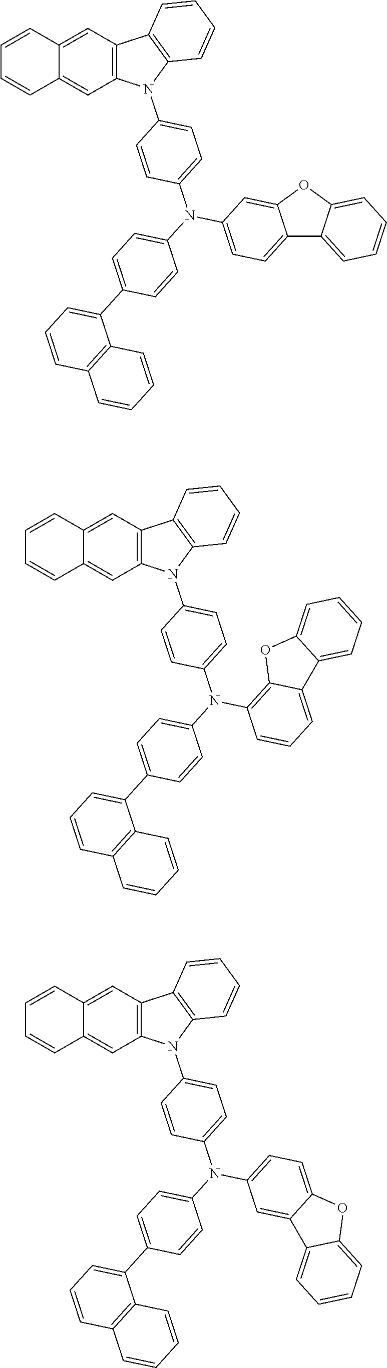 [SMILES: c1ccc2cc3c(cc2c1)c1ccccc1n3-c1ccc(N(c2ccc(-c3cccc4ccccc34)cc2)c2ccc3c(c2)oc2ccccc23)cc1 ; c1ccc2cc3c(cc2c1)c1ccccc1n3-c1ccc(N(c2ccc(-c3cccc4ccccc34)cc2)c2cccc3c2oc2ccccc23)cc1 ; c1ccc2cc3c(cc2c1)c1ccccc1n3-c1ccc(N(c2ccc(-c3cccc4ccccc34)cc2)c2ccc3oc4ccccc4c3c2)cc1] 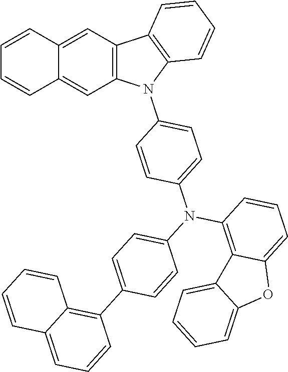 [SMILES: c1ccc2cc3c(cc2c1)c1ccccc1n3-c1ccc(N(c2ccc(-c3cccc4ccccc34)cc2)c2cccc3oc4ccccc4c23)cc1] 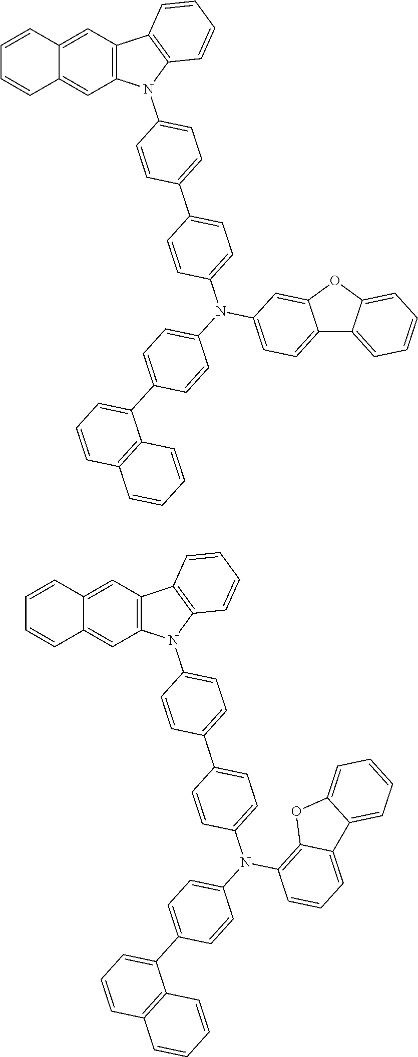 [SMILES: c1ccc2cc3c(cc2c1)c1ccccc1n3-c1ccc(-c2ccc(N(c3ccc(-c4cccc5ccccc45)cc3)c3ccc4c(c3)oc3ccccc34)cc2)cc1 ; c1ccc2cc3c(cc2c1)c1ccccc1n3-c1ccc(-c2ccc(N(c3ccc(-c4cccc5ccccc45)cc3)c3cccc4c3oc3ccccc34)cc2)cc1] 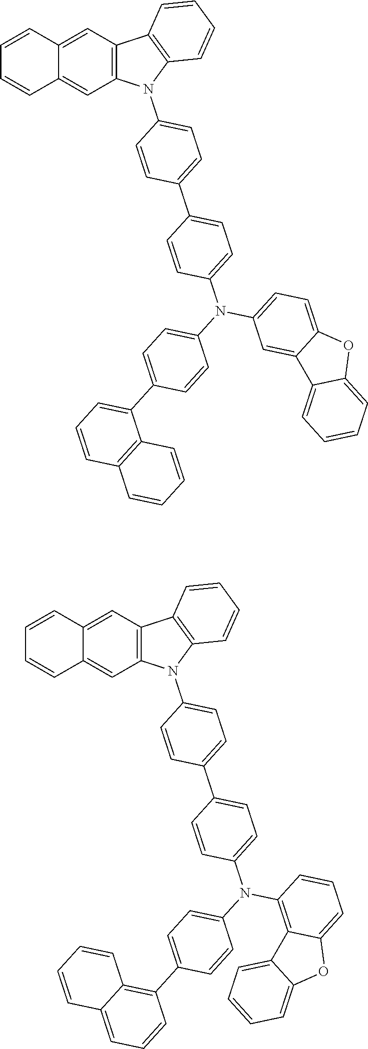 [SMILES: c1ccc2cc3c(cc2c1)c1ccccc1n3-c1ccc(-c2ccc(N(c3ccc(-c4cccc5ccccc45)cc3)c3ccc4oc5ccccc5c4c3)cc2)cc1 ; c1ccc2cc3c(cc2c1)c1ccccc1n3-c1ccc(-c2ccc(N(c3ccc(-c4cccc5ccccc45)cc3)c3cccc4oc5ccccc5c34)cc2)cc1] 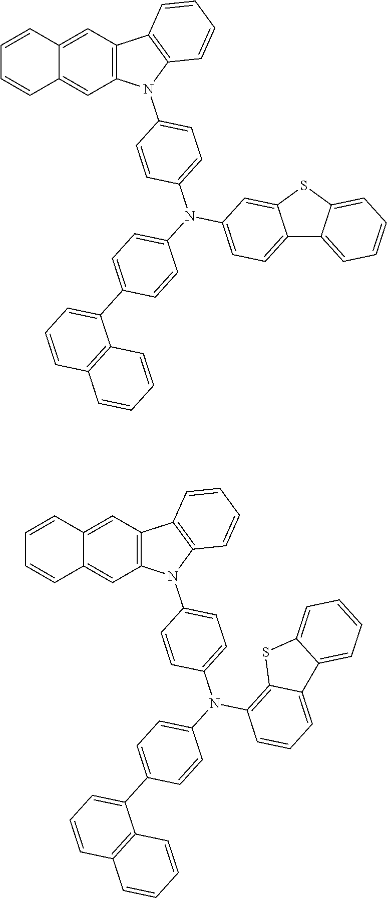 [SMILES: c1ccc2cc3c(cc2c1)c1ccccc1n3-c1ccc(N(c2ccc(-c3cccc4ccccc34)cc2)c2ccc3c(c2)sc2ccccc23)cc1 ; c1ccc2cc3c(cc2c1)c1ccccc1n3-c1ccc(N(c2ccc(-c3cccc4ccccc34)cc2)c2cccc3c2sc2ccccc23)cc1] 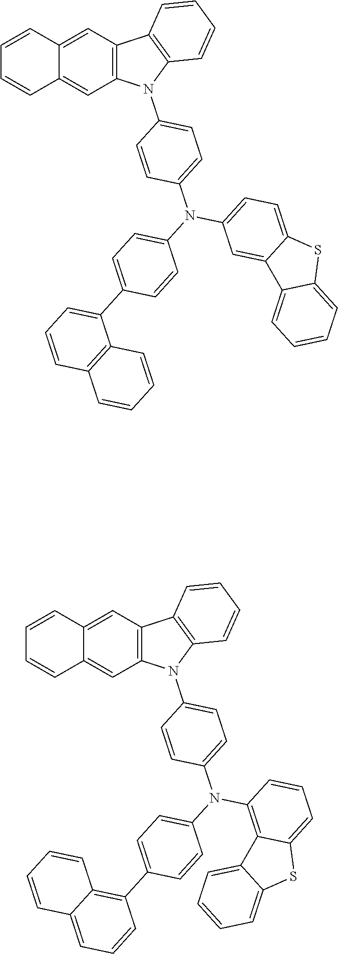 [SMILES: c1ccc2cc3c(cc2c1)c1ccccc1n3-c1ccc(N(c2ccc(-c3cccc4ccccc34)cc2)c2ccc3sc4ccccc4c3c2)cc1 ; c1ccc2cc3c(cc2c1)c1ccccc1n3-c1ccc(N(c2ccc(-c3cccc4ccccc34)cc2)c2cccc3sc4ccccc4c23)cc1] 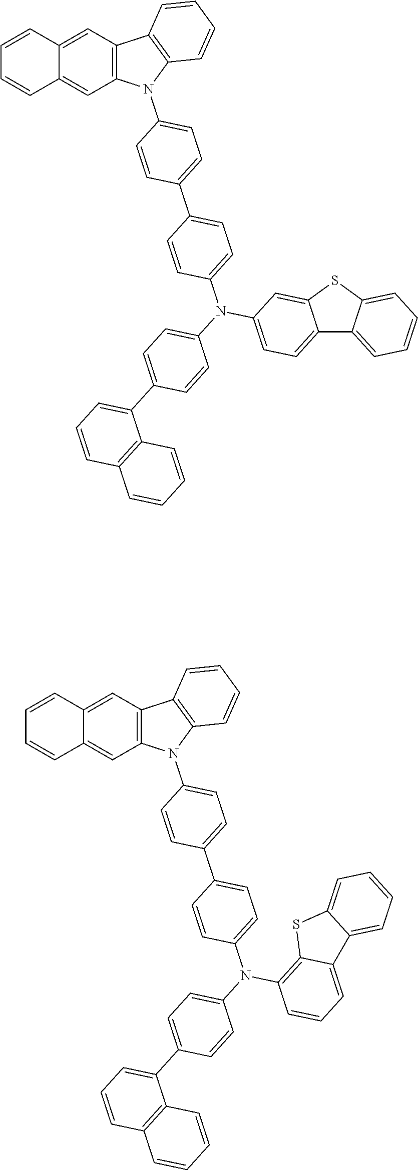 [SMILES: c1ccc2cc3c(cc2c1)c1ccccc1n3-c1ccc(-c2ccc(N(c3ccc(-c4cccc5ccccc45)cc3)c3ccc4c(c3)sc3ccccc34)cc2)cc1 ; c1ccc2cc3c(cc2c1)c1ccccc1n3-c1ccc(-c2ccc(N(c3ccc(-c4cccc5ccccc45)cc3)c3cccc4c3sc3ccccc34)cc2)cc1] 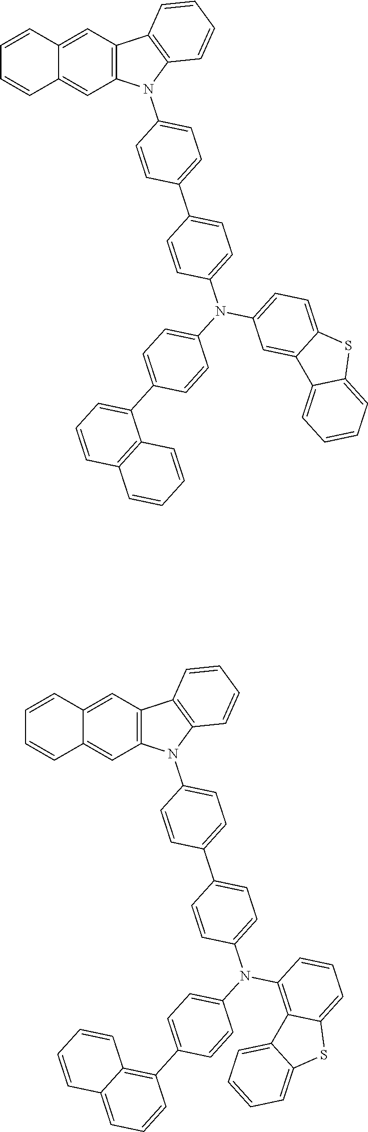 [SMILES: c1ccc2cc3c(cc2c1)c1ccccc1n3-c1ccc(-c2ccc(N(c3ccc(-c4cccc5ccccc45)cc3)c3ccc4sc5ccccc5c4c3)cc2)cc1 ; c1ccc2cc3c(cc2c1)c1ccccc1n3-c1ccc(-c2ccc(N(c3ccc(-c4cccc5ccccc45)cc3)c3cccc4sc5ccccc5c34)cc2)cc1] 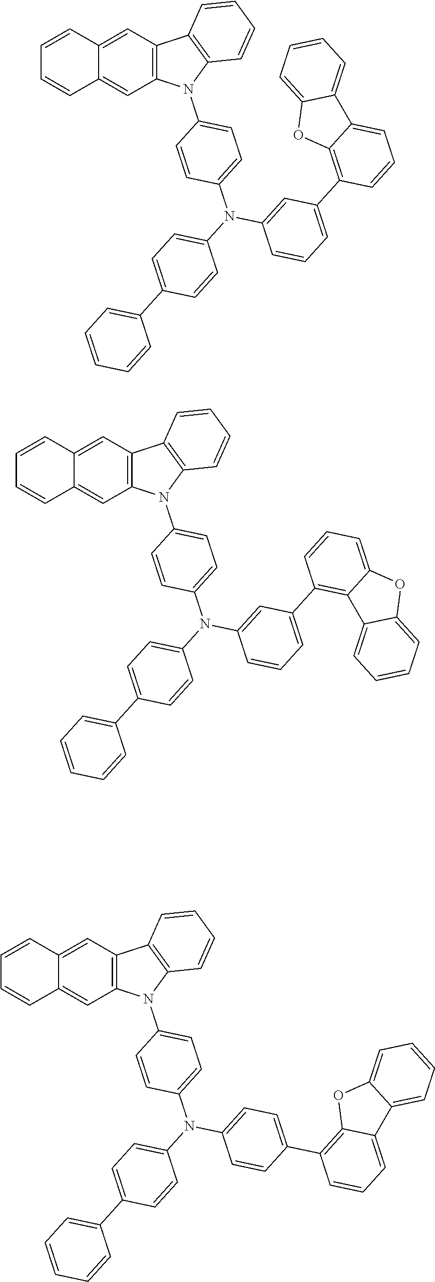 [SMILES: c1ccc(-c2ccc(N(c3ccc(-n4c5ccccc5c5cc6ccccc6cc54)cc3)c3cccc(-c4cccc5c4oc4ccccc45)c3)cc2)cc1 ; c1ccc(-c2ccc(N(c3ccc(-n4c5ccccc5c5cc6ccccc6cc54)cc3)c3cccc(-c4cccc5oc6ccccc6c45)c3)cc2)cc1 ; c1ccc(-c2ccc(N(c3ccc(-c4cccc5c4oc4ccccc45)cc3)c3ccc(-n4c5ccccc5c5cc6ccccc6cc54)cc3)cc2)cc1] 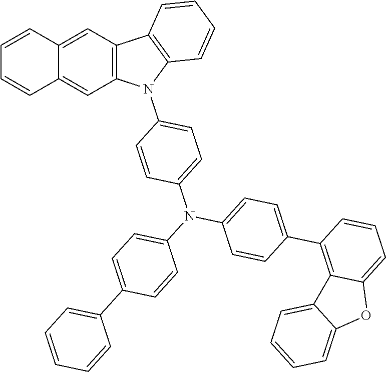 [SMILES: c1ccc(-c2ccc(N(c3ccc(-c4cccc5oc6ccccc6c45)cc3)c3ccc(-n4c5ccccc5c5cc6ccccc6cc54)cc3)cc2)cc1] 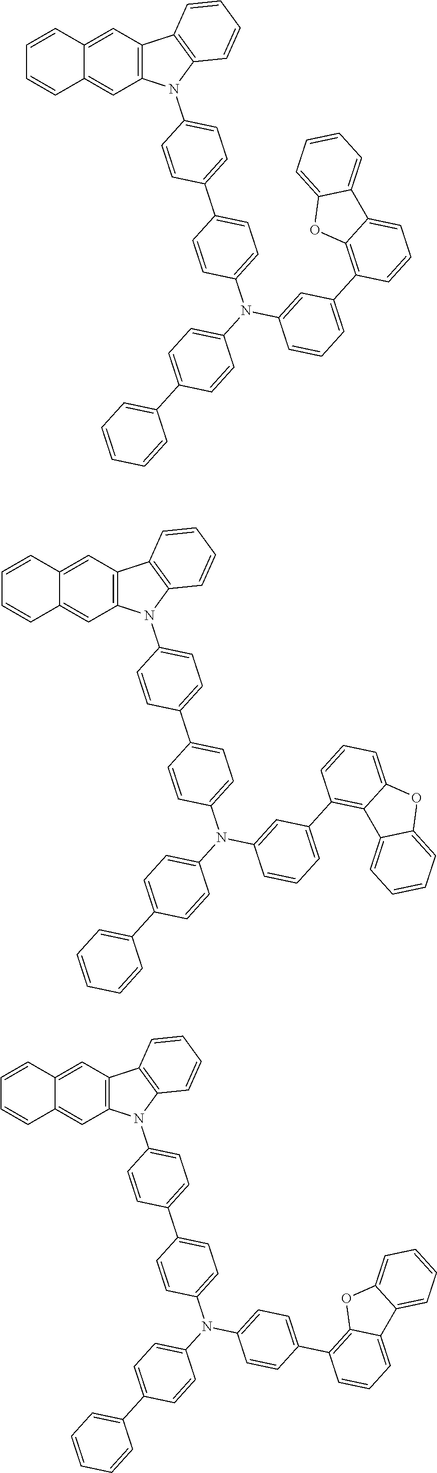 [SMILES: c1ccc(-c2ccc(N(c3ccc(-c4ccc(-n5c6ccccc6c6cc7ccccc7cc65)cc4)cc3)c3cccc(-c4cccc5c4oc4ccccc45)c3)cc2)cc1 ; c1ccc(-c2ccc(N(c3ccc(-c4ccc(-n5c6ccccc6c6cc7ccccc7cc65)cc4)cc3)c3cccc(-c4cccc5oc6ccccc6c45)c3)cc2)cc1 ; c1ccc(-c2ccc(N(c3ccc(-c4ccc(-n5c6ccccc6c6cc7ccccc7cc65)cc4)cc3)c3ccc(-c4cccc5c4oc4ccccc45)cc3)cc2)cc1] 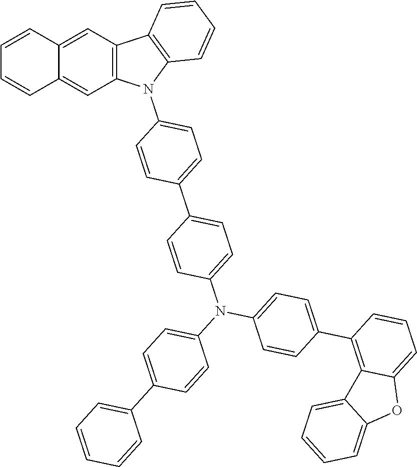 [SMILES: c1ccc(-c2ccc(N(c3ccc(-c4ccc(-n5c6ccccc6c6cc7ccccc7cc65)cc4)cc3)c3ccc(-c4cccc5oc6ccccc6c45)cc3)cc2)cc1] 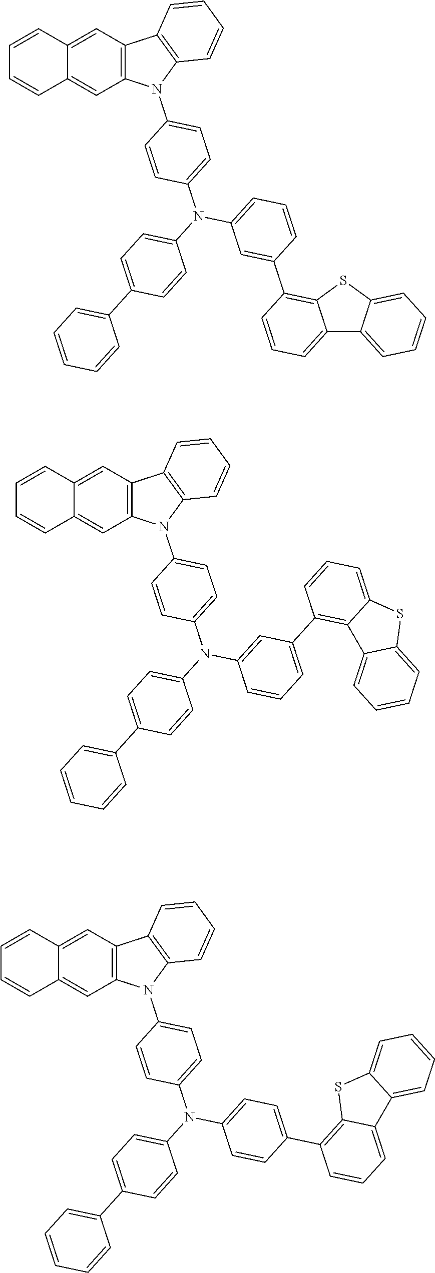 [SMILES: c1ccc(-c2ccc(N(c3ccc(-n4c5ccccc5c5cc6ccccc6cc54)cc3)c3cccc(-c4cccc5c4sc4ccccc45)c3)cc2)cc1 ; c1ccc(-c2ccc(N(c3ccc(-n4c5ccccc5c5cc6ccccc6cc54)cc3)c3cccc(-c4cccc5sc6ccccc6c45)c3)cc2)cc1 ; c1ccc(-c2ccc(N(c3ccc(-c4cccc5c4sc4ccccc45)cc3)c3ccc(-n4c5ccccc5c5cc6ccccc6cc54)cc3)cc2)cc1] 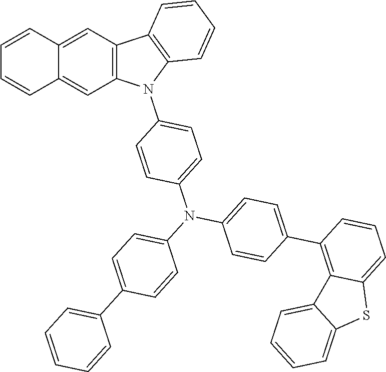 [SMILES: c1ccc(-c2ccc(N(c3ccc(-c4cccc5sc6ccccc6c45)cc3)c3ccc(-n4c5ccccc5c5cc6ccccc6cc54)cc3)cc2)cc1] 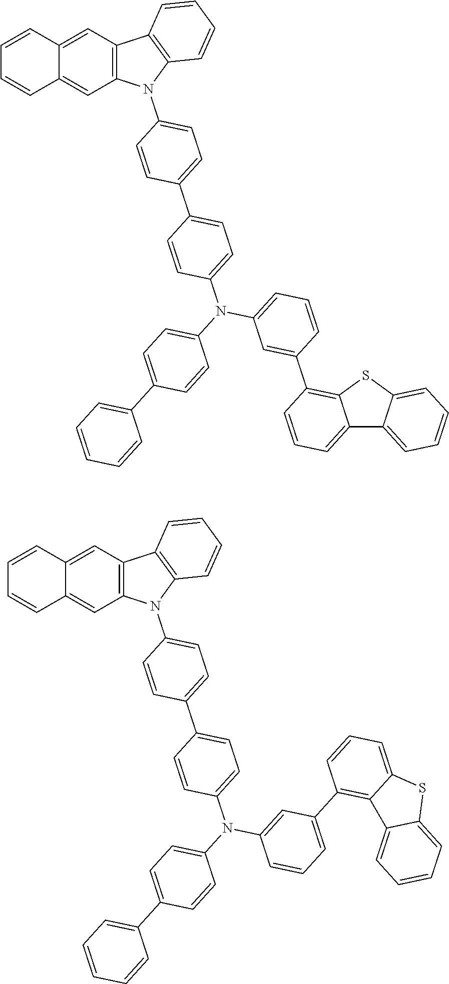 [SMILES: c1ccc(-c2ccc(N(c3ccc(-c4ccc(-n5c6ccccc6c6cc7ccccc7cc65)cc4)cc3)c3cccc(-c4cccc5c4sc4ccccc45)c3)cc2)cc1 ; c1ccc(-c2ccc(N(c3ccc(-c4ccc(-n5c6ccccc6c6cc7ccccc7cc65)cc4)cc3)c3cccc(-c4cccc5sc6ccccc6c45)c3)cc2)cc1] 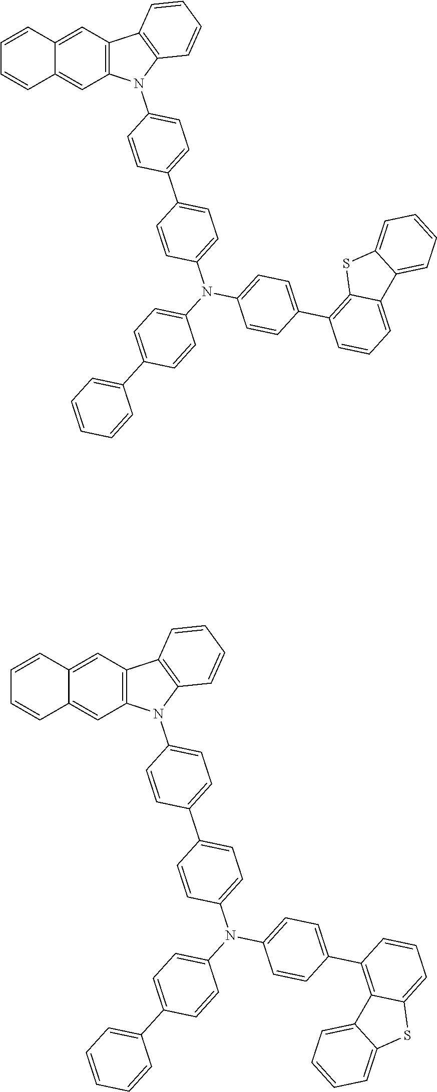 [SMILES: c1ccc(-c2ccc(N(c3ccc(-c4ccc(-n5c6ccccc6c6cc7ccccc7cc65)cc4)cc3)c3ccc(-c4cccc5c4sc4ccccc45)cc3)cc2)cc1 ; c1ccc(-c2ccc(N(c3ccc(-c4ccc(-n5c6ccccc6c6cc7ccccc7cc65)cc4)cc3)c3ccc(-c4cccc5sc6ccccc6c45)cc3)cc2)cc1] 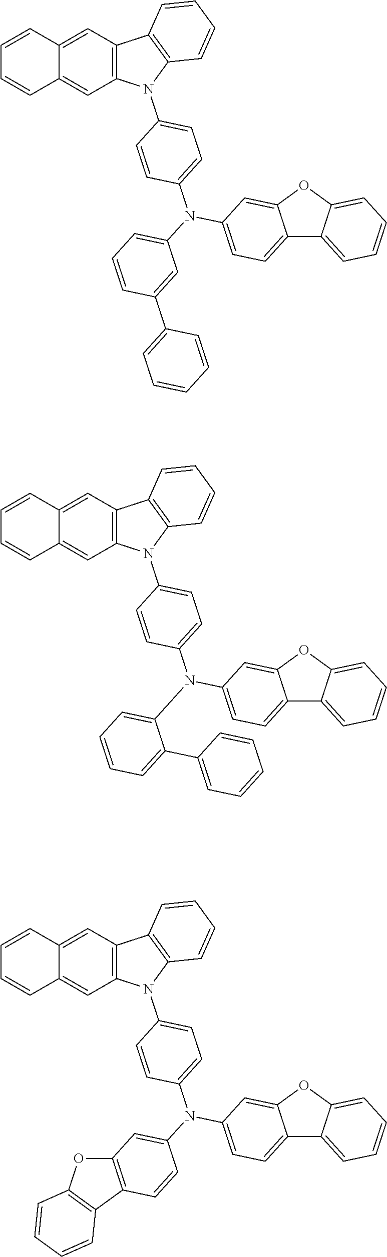 [SMILES: c1ccc(-c2cccc(N(c3ccc(-n4c5ccccc5c5cc6ccccc6cc54)cc3)c3ccc4c(c3)oc3ccccc34)c2)cc1 ; c1ccc(-c2ccccc2N(c2ccc(-n3c4ccccc4c4cc5ccccc5cc43)cc2)c2ccc3c(c2)oc2ccccc23)cc1 ; c1ccc2cc3c(cc2c1)c1ccccc1n3-c1ccc(N(c2ccc3c(c2)oc2ccccc23)c2ccc3c(c2)oc2ccccc23)cc1] 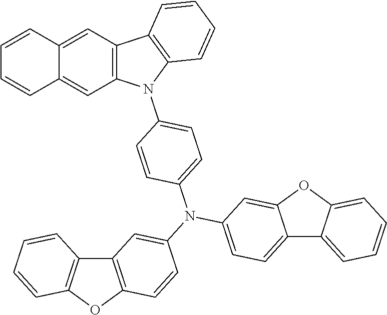 [SMILES: c1ccc2cc3c(cc2c1)c1ccccc1n3-c1ccc(N(c2ccc3c(c2)oc2ccccc23)c2ccc3oc4ccccc4c3c2)cc1] 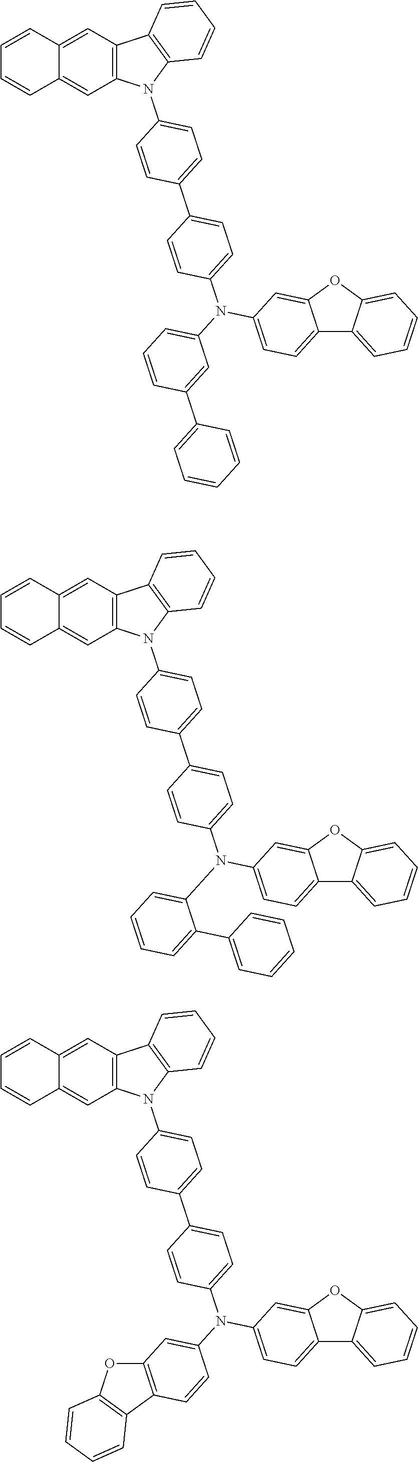 [SMILES: c1ccc(-c2cccc(N(c3ccc(-c4ccc(-n5c6ccccc6c6cc7ccccc7cc65)cc4)cc3)c3ccc4c(c3)oc3ccccc34)c2)cc1 ; c1ccc(-c2ccccc2N(c2ccc(-c3ccc(-n4c5ccccc5c5cc6ccccc6cc54)cc3)cc2)c2ccc3c(c2)oc2ccccc23)cc1 ; c1ccc2cc3c(cc2c1)c1ccccc1n3-c1ccc(-c2ccc(N(c3ccc4c(c3)oc3ccccc34)c3ccc4c(c3)oc3ccccc34)cc2)cc1] 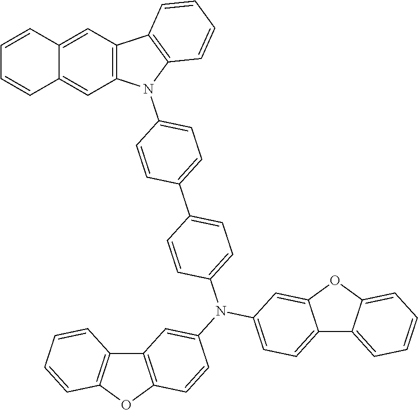 [SMILES: c1ccc2cc3c(cc2c1)c1ccccc1n3-c1ccc(-c2ccc(N(c3ccc4c(c3)oc3ccccc34)c3ccc4oc5ccccc5c4c3)cc2)cc1] 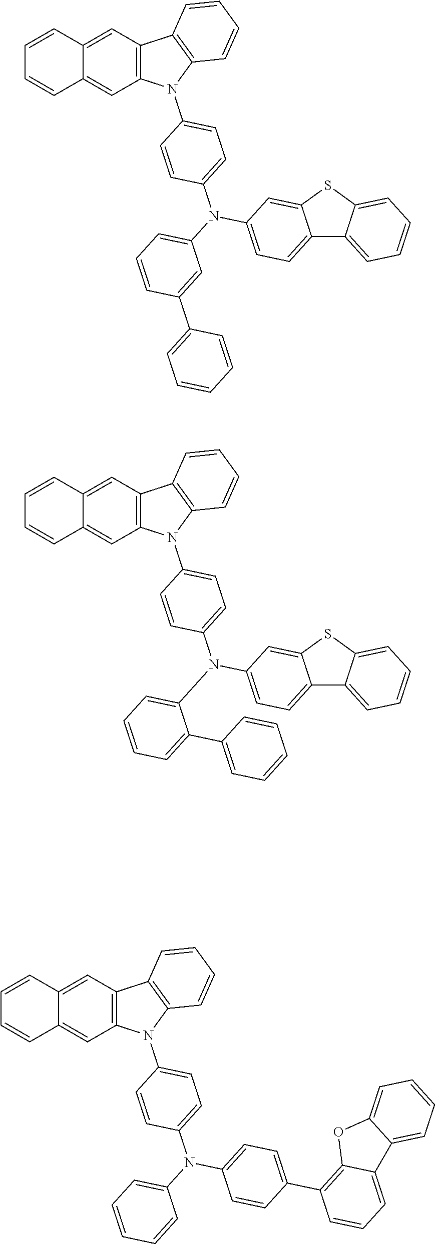 [SMILES: c1ccc(-c2cccc(N(c3ccc(-n4c5ccccc5c5cc6ccccc6cc54)cc3)c3ccc4c(c3)sc3ccccc34)c2)cc1 ; c1ccc(-c2ccccc2N(c2ccc(-n3c4ccccc4c4cc5ccccc5cc43)cc2)c2ccc3c(c2)sc2ccccc23)cc1 ; c1ccc(N(c2ccc(-c3cccc4c3oc3ccccc34)cc2)c2ccc(-n3c4ccccc4c4cc5ccccc5cc43)cc2)cc1] 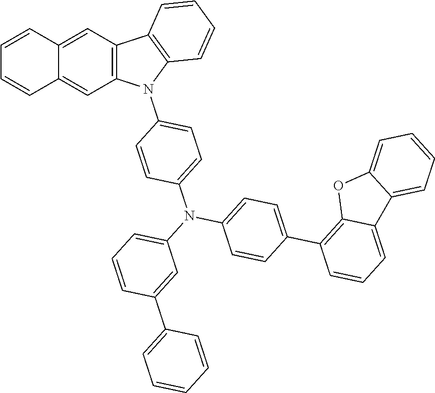 [SMILES: c1ccc(-c2cccc(N(c3ccc(-c4cccc5c4oc4ccccc45)cc3)c3ccc(-n4c5ccccc5c5cc6ccccc6cc54)cc3)c2)cc1] 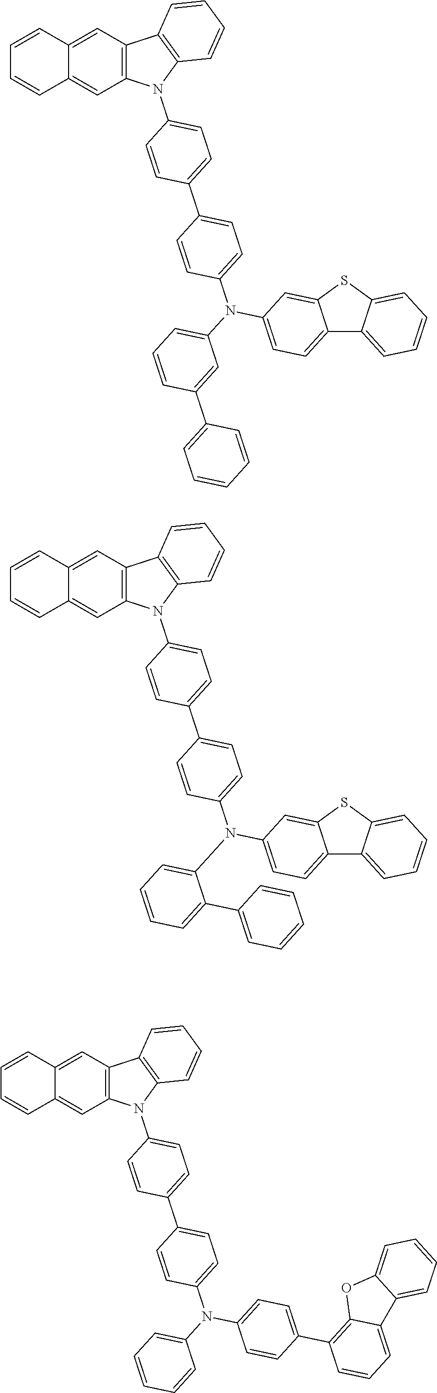 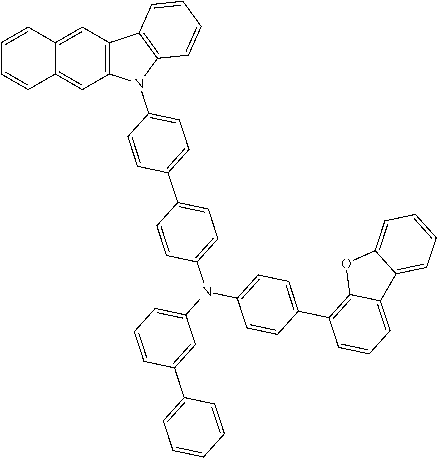 [SMILES: c1ccc(-c2cccc(N(c3ccc(-c4ccc(-n5c6ccccc6c6cc7ccccc7cc65)cc4)cc3)c3ccc(-c4cccc5c4oc4ccccc45)cc3)c2)cc1] 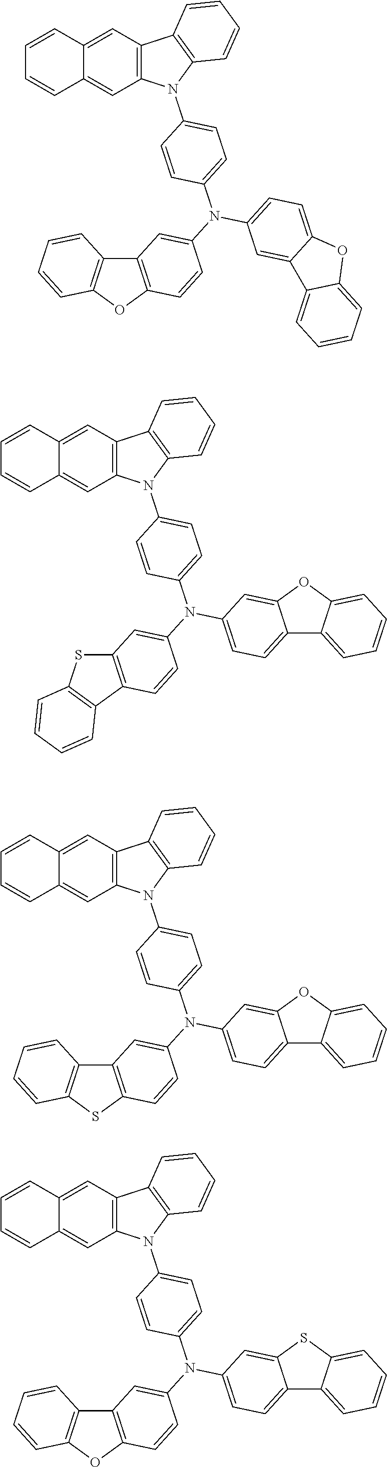 [SMILES: c1ccc2cc3c(cc2c1)c1ccccc1n3-c1ccc(N(c2ccc3oc4ccccc4c3c2)c2ccc3oc4ccccc4c3c2)cc1 ; c1ccc2cc3c(cc2c1)c1ccccc1n3-c1ccc(N(c2ccc3c(c2)oc2ccccc23)c2ccc3c(c2)sc2ccccc23)cc1 ; c1ccc2cc3c(cc2c1)c1ccccc1n3-c1ccc(N(c2ccc3c(c2)oc2ccccc23)c2ccc3sc4ccccc4c3c2)cc1 ; c1ccc2cc3c(cc2c1)c1ccccc1n3-c1ccc(N(c2ccc3c(c2)sc2ccccc23)c2ccc3oc4ccccc4c3c2)cc1] 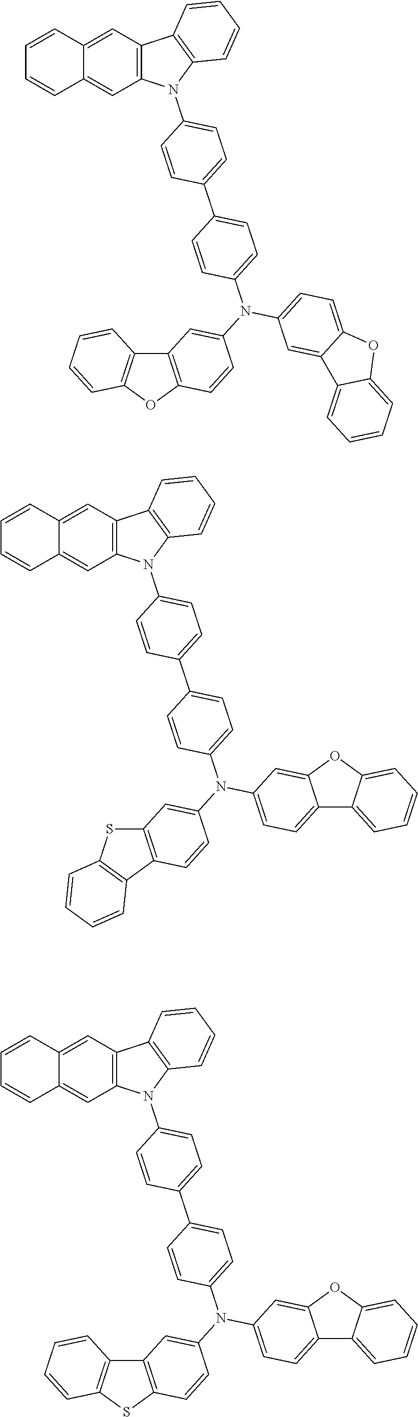 [SMILES: c1ccc2cc3c(cc2c1)c1ccccc1n3-c1ccc(-c2ccc(N(c3ccc4oc5ccccc5c4c3)c3ccc4oc5ccccc5c4c3)cc2)cc1 ; c1ccc2cc3c(cc2c1)c1ccccc1n3-c1ccc(-c2ccc(N(c3ccc4c(c3)oc3ccccc34)c3ccc4c(c3)sc3ccccc34)cc2)cc1 ; c1ccc2cc3c(cc2c1)c1ccccc1n3-c1ccc(-c2ccc(N(c3ccc4c(c3)oc3ccccc34)c3ccc4sc5ccccc5c4c3)cc2)cc1] 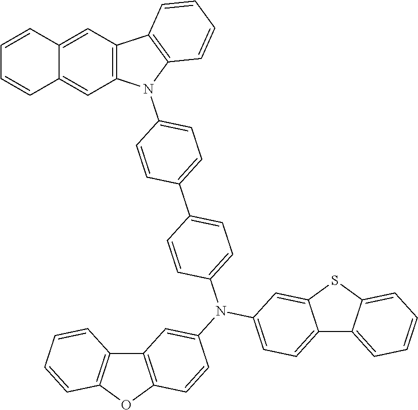 [SMILES: c1ccc2cc3c(cc2c1)c1ccccc1n3-c1ccc(-c2ccc(N(c3ccc4c(c3)sc3ccccc34)c3ccc4oc5ccccc5c4c3)cc2)cc1] 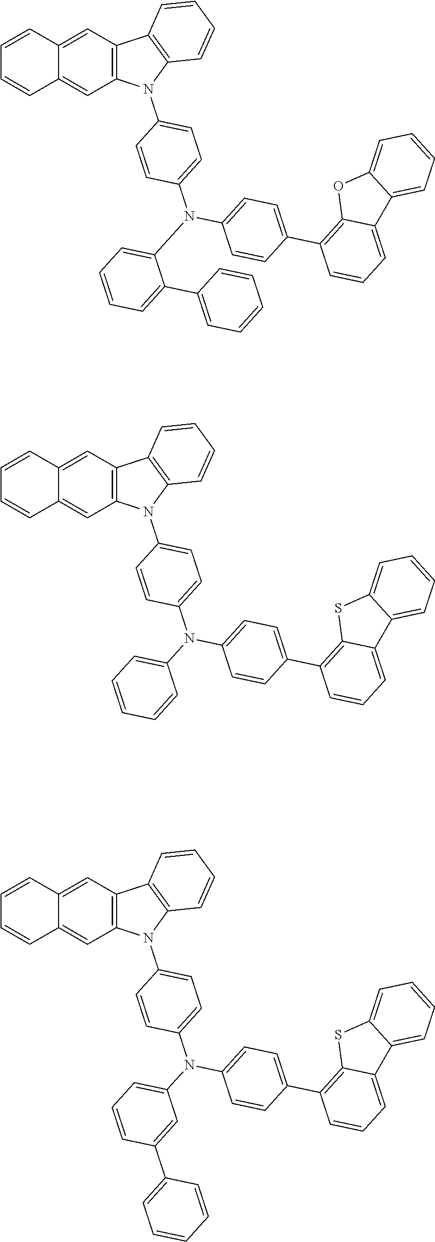 [SMILES: c1ccc(-c2ccccc2N(c2ccc(-c3cccc4c3oc3ccccc34)cc2)c2ccc(-n3c4ccccc4c4cc5ccccc5cc43)cc2)cc1 ; c1ccc(N(c2ccc(-c3cccc4c3sc3ccccc34)cc2)c2ccc(-n3c4ccccc4c4cc5ccccc5cc43)cc2)cc1 ; c1ccc(-c2cccc(N(c3ccc(-c4cccc5c4sc4ccccc45)cc3)c3ccc(-n4c5ccccc5c5cc6ccccc6cc54)cc3)c2)cc1] 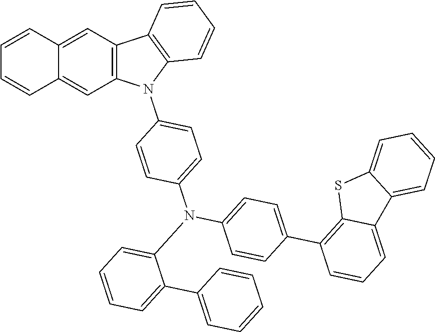 [SMILES: c1ccc(-c2ccccc2N(c2ccc(-c3cccc4c3sc3ccccc34)cc2)c2ccc(-n3c4ccccc4c4cc5ccccc5cc43)cc2)cc1] 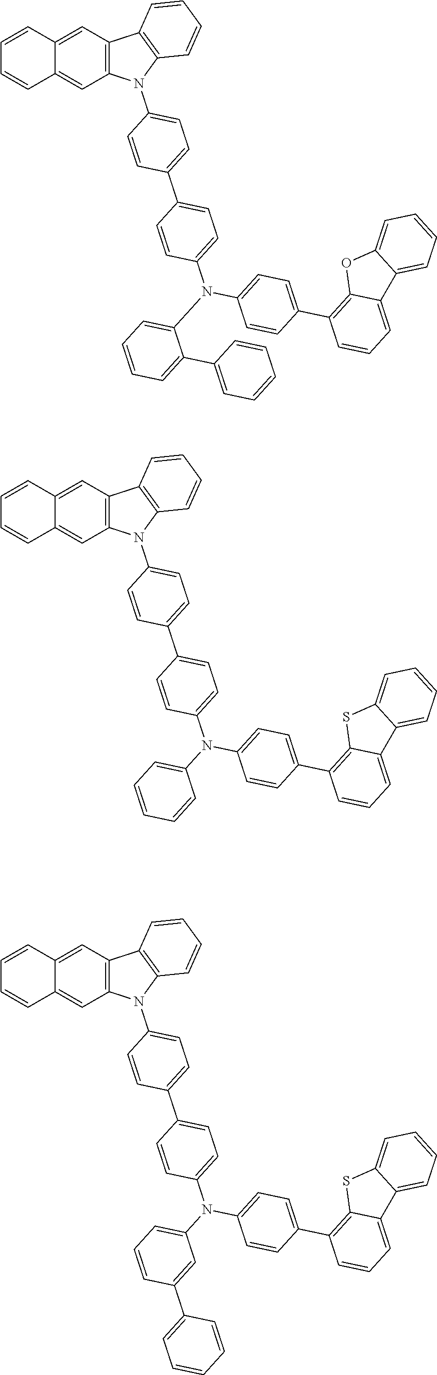 [SMILES: c1ccc(-c2ccccc2N(c2ccc(-c3ccc(-n4c5ccccc5c5cc6ccccc6cc54)cc3)cc2)c2ccc(-c3cccc4c3oc3ccccc34)cc2)cc1 ; c1ccc(N(c2ccc(-c3ccc(-n4c5ccccc5c5cc6ccccc6cc54)cc3)cc2)c2ccc(-c3cccc4c3sc3ccccc34)cc2)cc1 ; c1ccc(-c2cccc(N(c3ccc(-c4ccc(-n5c6ccccc6c6cc7ccccc7cc65)cc4)cc3)c3ccc(-c4cccc5c4sc4ccccc45)cc3)c2)cc1] 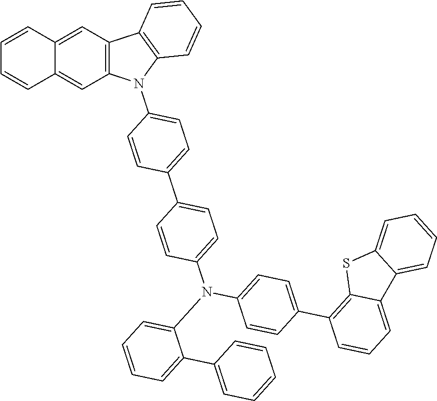 [SMILES: c1ccc(-c2ccccc2N(c2ccc(-c3ccc(-n4c5ccccc5c5cc6ccccc6cc54)cc3)cc2)c2ccc(-c3cccc4c3sc3ccccc34)cc2)cc1] 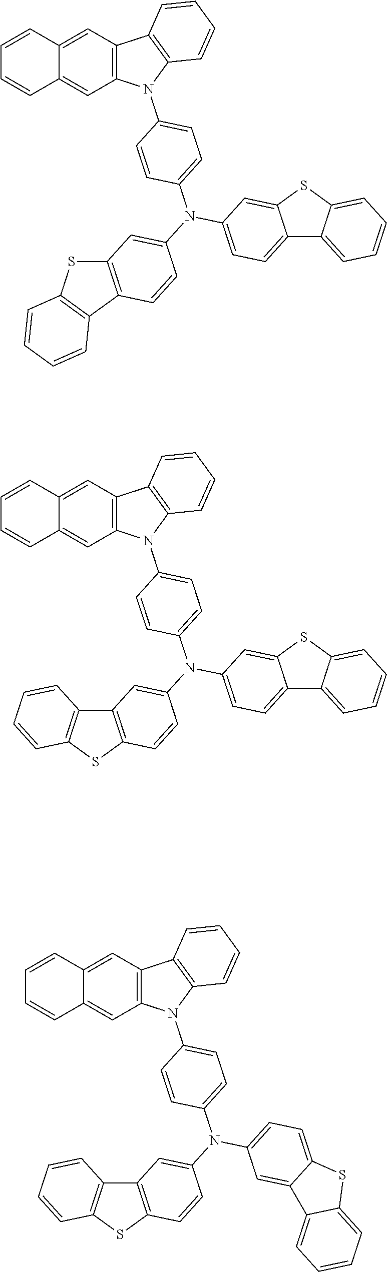 [SMILES: c1ccc2cc3c(cc2c1)c1ccccc1n3-c1ccc(N(c2ccc3c(c2)sc2ccccc23)c2ccc3c(c2)sc2ccccc23)cc1 ; c1ccc2cc3c(cc2c1)c1ccccc1n3-c1ccc(N(c2ccc3c(c2)sc2ccccc23)c2ccc3sc4ccccc4c3c2)cc1 ; c1ccc2cc3c(cc2c1)c1ccccc1n3-c1ccc(N(c2ccc3sc4ccccc4c3c2)c2ccc3sc4ccccc4c3c2)cc1] 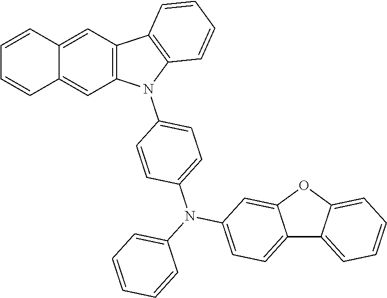 [SMILES: c1ccc(N(c2ccc(-n3c4ccccc4c4cc5ccccc5cc43)cc2)c2ccc3c(c2)oc2ccccc23)cc1] 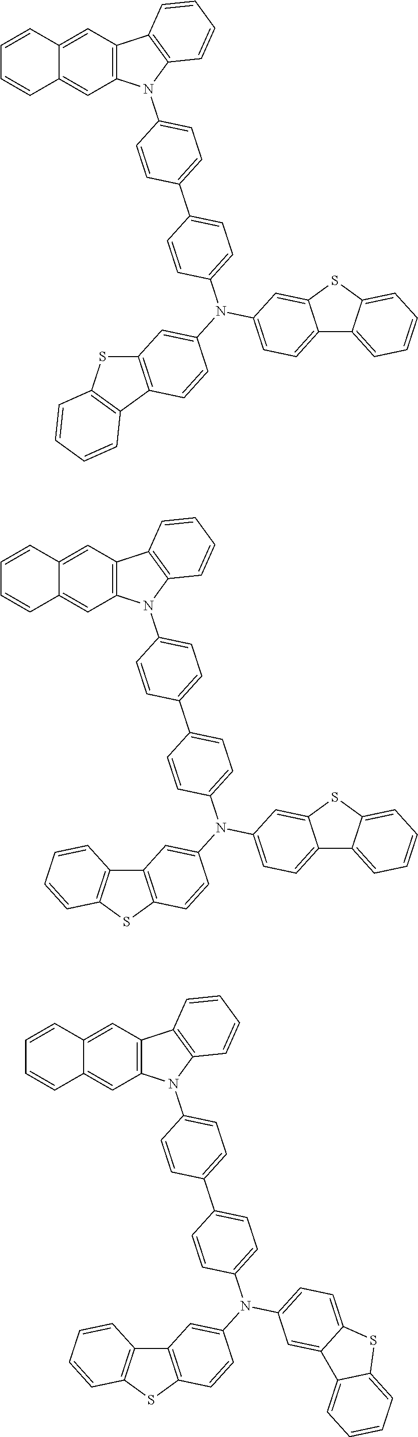 [SMILES: c1ccc2cc3c(cc2c1)c1ccccc1n3-c1ccc(-c2ccc(N(c3ccc4c(c3)sc3ccccc34)c3ccc4c(c3)sc3ccccc34)cc2)cc1 ; c1ccc2cc3c(cc2c1)c1ccccc1n3-c1ccc(-c2ccc(N(c3ccc4c(c3)sc3ccccc34)c3ccc4sc5ccccc5c4c3)cc2)cc1 ; c1ccc2cc3c(cc2c1)c1ccccc1n3-c1ccc(-c2ccc(N(c3ccc4sc5ccccc5c4c3)c3ccc4sc5ccccc5c4c3)cc2)cc1] 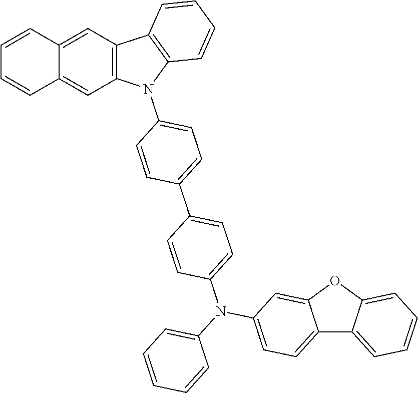 [SMILES: c1ccc(N(c2ccc(-c3ccc(-n4c5ccccc5c5cc6ccccc6cc54)cc3)cc2)c2ccc3c(c2)oc2ccccc23)cc1] 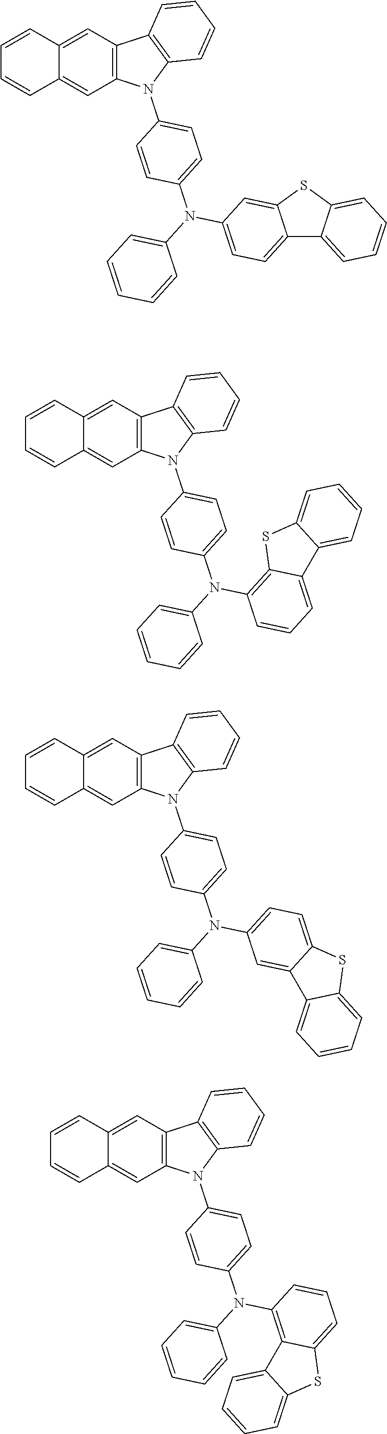 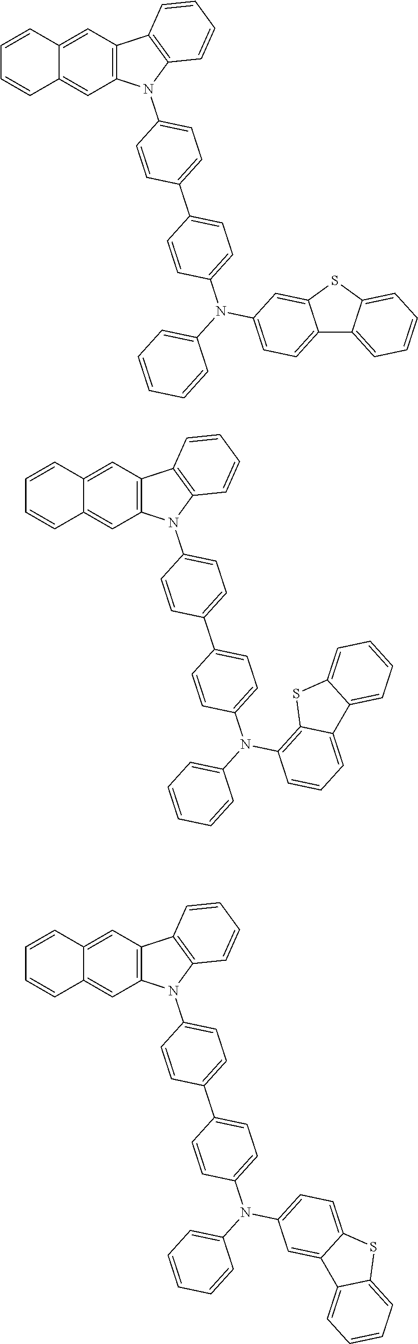 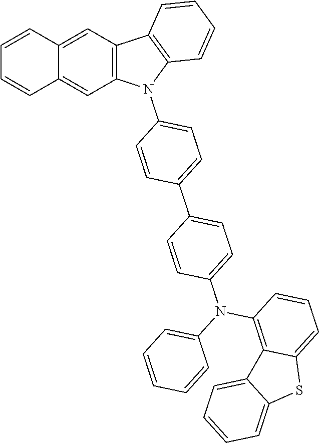 [SMILES: c1ccc(N(c2ccc(-c3ccc(-n4c5ccccc5c5cc6ccccc6cc54)cc3)cc2)c2cccc3sc4ccccc4c23)cc1] 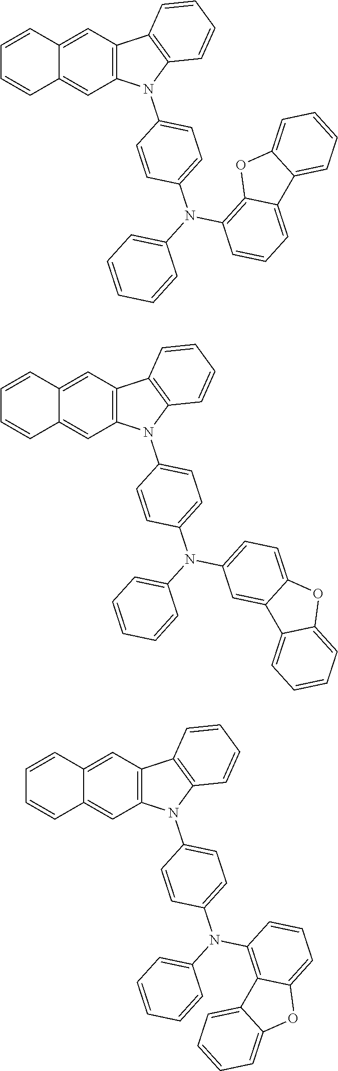 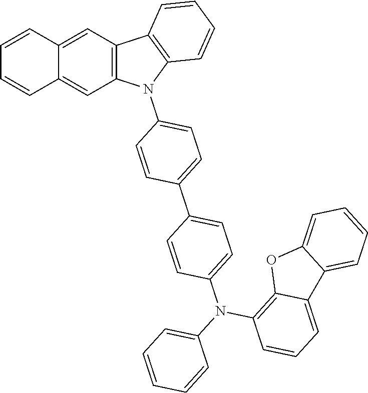 [SMILES: c1ccc(N(c2ccc(-c3ccc(-n4c5ccccc5c5cc6ccccc6cc54)cc3)cc2)c2cccc3c2oc2ccccc23)cc1] 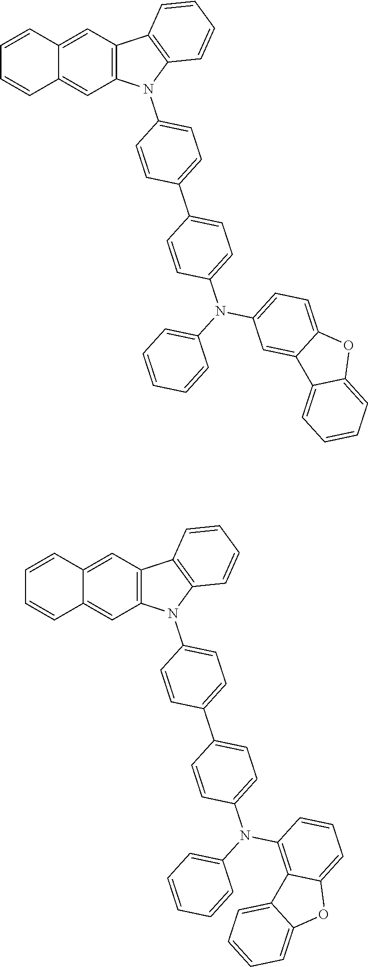 [SMILES: c1ccc(N(c2ccc(-c3ccc(-n4c5ccccc5c5cc6ccccc6cc54)cc3)cc2)c2ccc3oc4ccccc4c3c2)cc1 ; c1ccc(N(c2ccc(-c3ccc(-n4c5ccccc5c5cc6ccccc6cc54)cc3)cc2)c2cccc3oc4ccccc4c23)cc1] 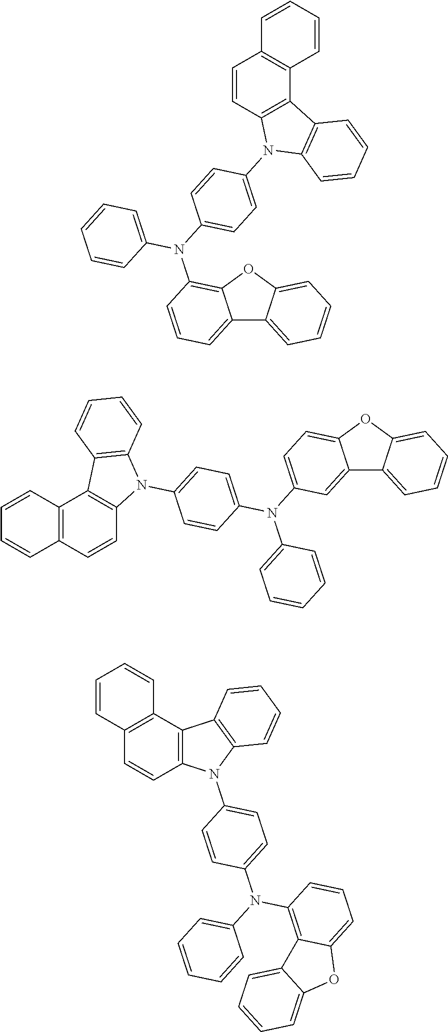 [SMILES: c1ccc(N(c2ccc(-n3c4ccccc4c4c5ccccc5ccc43)cc2)c2cccc3c2oc2ccccc23)cc1 ; c1ccc(N(c2ccc(-n3c4ccccc4c4c5ccccc5ccc43)cc2)c2ccc3oc4ccccc4c3c2)cc1 ; c1ccc(N(c2ccc(-n3c4ccccc4c4c5ccccc5ccc43)cc2)c2cccc3oc4ccccc4c23)cc1] 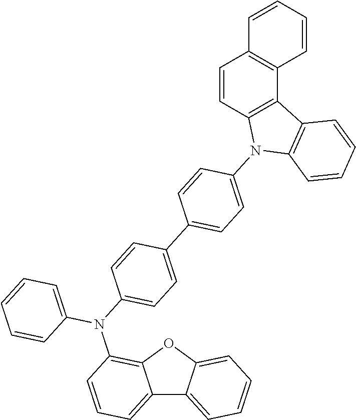 [SMILES: c1ccc(N(c2ccc(-c3ccc(-n4c5ccccc5c5c6ccccc6ccc54)cc3)cc2)c2cccc3c2oc2ccccc23)cc1] 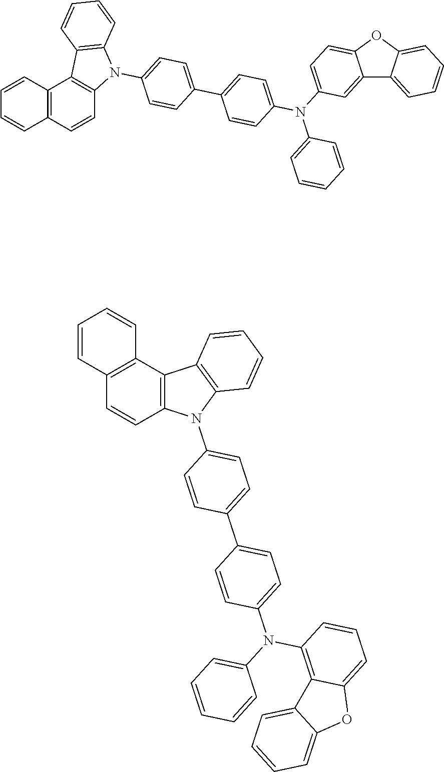 [SMILES: c1ccc(N(c2ccc(-c3ccc(-n4c5ccccc5c5c6ccccc6ccc54)cc3)cc2)c2ccc3oc4ccccc4c3c2)cc1 ; c1ccc(N(c2ccc(-c3ccc(-n4c5ccccc5c5c6ccccc6ccc54)cc3)cc2)c2cccc3oc4ccccc4c23)cc1] 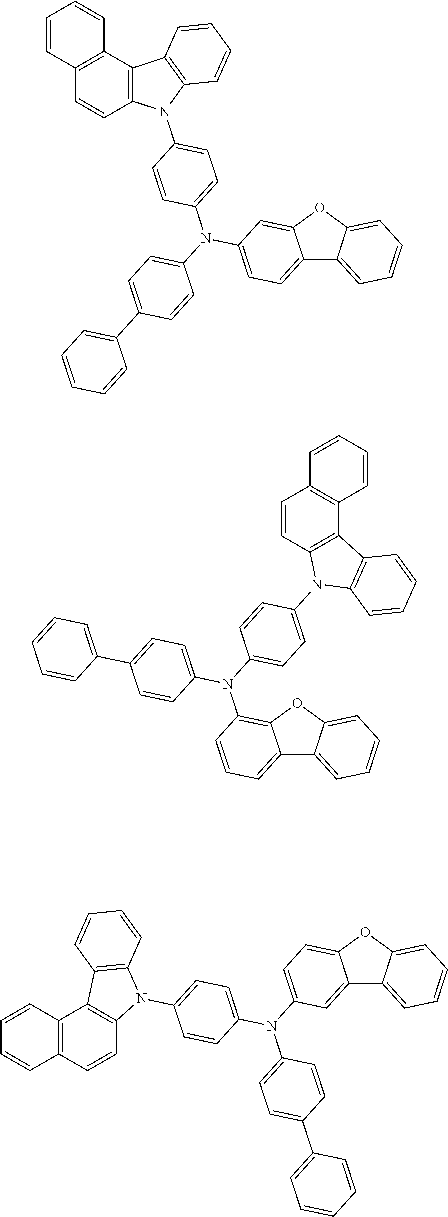 [SMILES: c1ccc(-c2ccc(N(c3ccc(-n4c5ccccc5c5c6ccccc6ccc54)cc3)c3ccc4c(c3)oc3ccccc34)cc2)cc1 ; c1ccc(-c2ccc(N(c3ccc(-n4c5ccccc5c5c6ccccc6ccc54)cc3)c3cccc4c3oc3ccccc34)cc2)cc1 ; c1ccc(-c2ccc(N(c3ccc(-n4c5ccccc5c5c6ccccc6ccc54)cc3)c3ccc4oc5ccccc5c4c3)cc2)cc1] 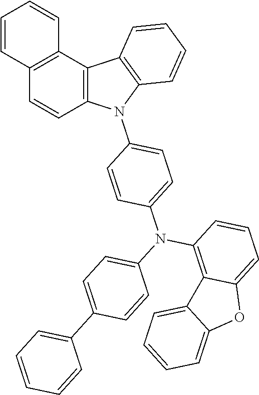 [SMILES: c1ccc(-c2ccc(N(c3ccc(-n4c5ccccc5c5c6ccccc6ccc54)cc3)c3cccc4oc5ccccc5c34)cc2)cc1] 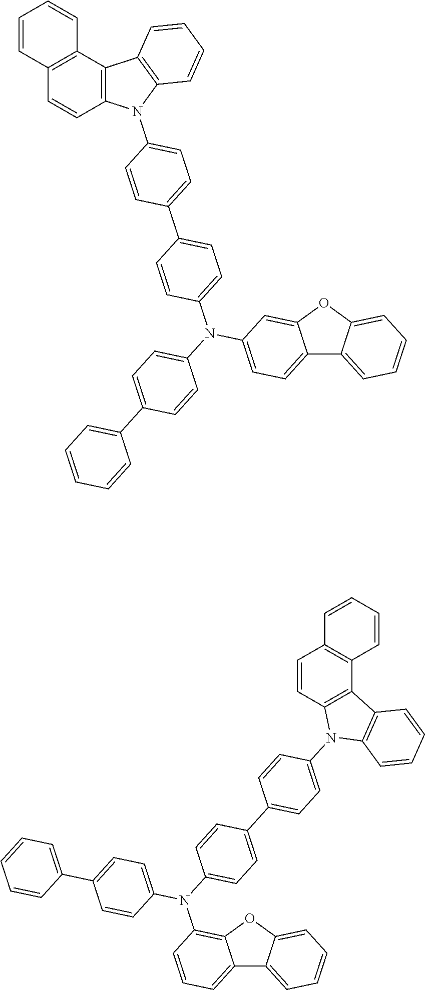 [SMILES: c1ccc(-c2ccc(N(c3ccc(-c4ccc(-n5c6ccccc6c6c7ccccc7ccc65)cc4)cc3)c3ccc4c(c3)oc3ccccc34)cc2)cc1 ; c1ccc(-c2ccc(N(c3ccc(-c4ccc(-n5c6ccccc6c6c7ccccc7ccc65)cc4)cc3)c3cccc4c3oc3ccccc34)cc2)cc1] 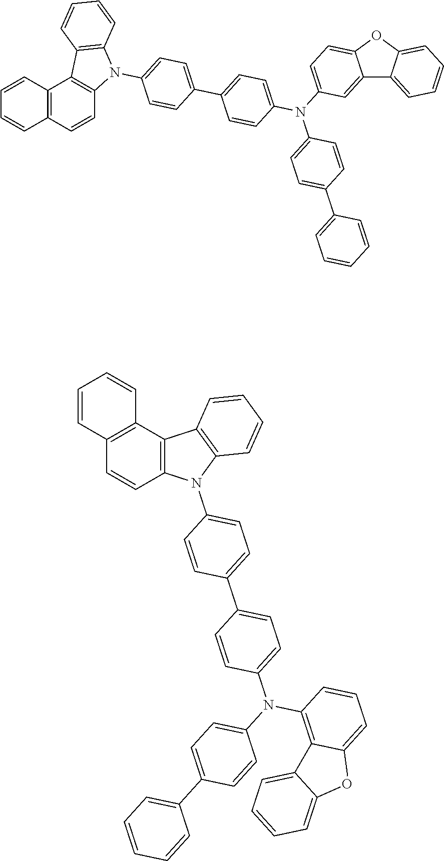 [SMILES: c1ccc(-c2ccc(N(c3ccc(-c4ccc(-n5c6ccccc6c6c7ccccc7ccc65)cc4)cc3)c3ccc4oc5ccccc5c4c3)cc2)cc1 ; c1ccc(-c2ccc(N(c3ccc(-c4ccc(-n5c6ccccc6c6c7ccccc7ccc65)cc4)cc3)c3cccc4oc5ccccc5c34)cc2)cc1] 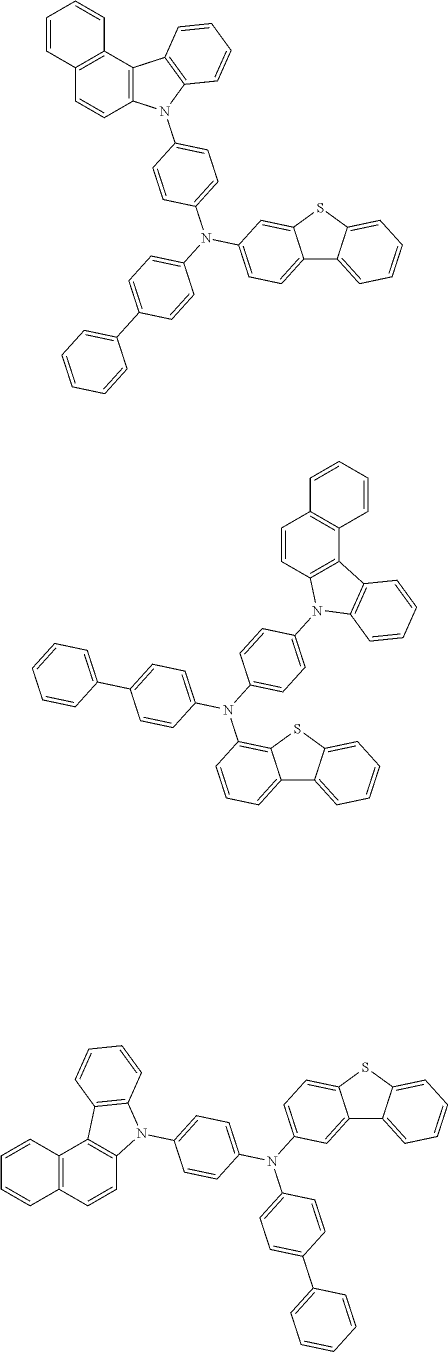 [SMILES: c1ccc(-c2ccc(N(c3ccc(-n4c5ccccc5c5c6ccccc6ccc54)cc3)c3ccc4c(c3)sc3ccccc34)cc2)cc1 ; c1ccc(-c2ccc(N(c3ccc(-n4c5ccccc5c5c6ccccc6ccc54)cc3)c3cccc4c3sc3ccccc34)cc2)cc1 ; c1ccc(-c2ccc(N(c3ccc(-n4c5ccccc5c5c6ccccc6ccc54)cc3)c3ccc4sc5ccccc5c4c3)cc2)cc1] 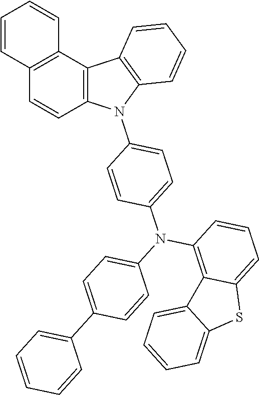 [SMILES: c1ccc(-c2ccc(N(c3ccc(-n4c5ccccc5c5c6ccccc6ccc54)cc3)c3cccc4sc5ccccc5c34)cc2)cc1] 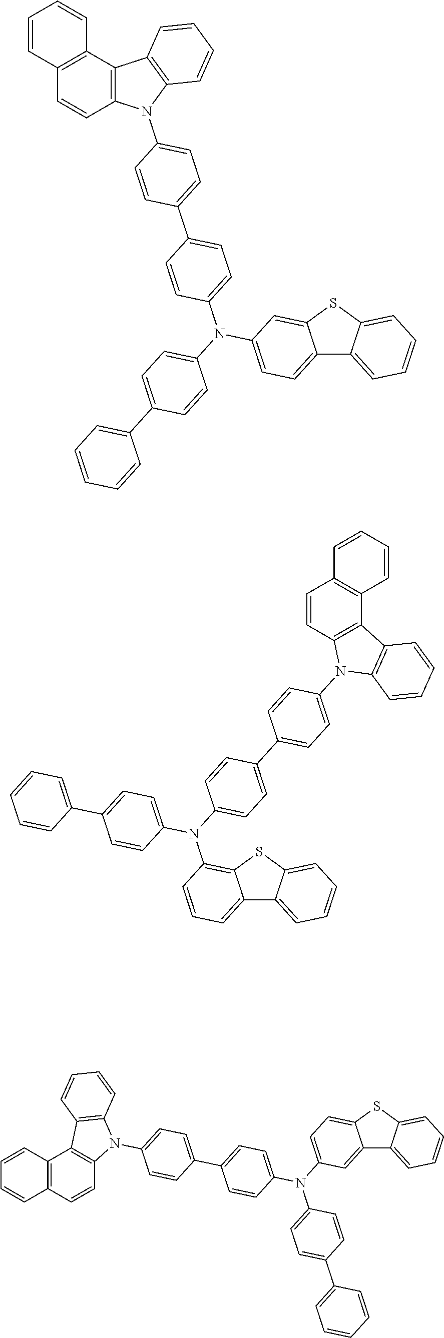 [SMILES: c1ccc(-c2ccc(N(c3ccc(-c4ccc(-n5c6ccccc6c6c7ccccc7ccc65)cc4)cc3)c3ccc4c(c3)sc3ccccc34)cc2)cc1 ; c1ccc(-c2ccc(N(c3ccc(-c4ccc(-n5c6ccccc6c6c7ccccc7ccc65)cc4)cc3)c3cccc4c3sc3ccccc34)cc2)cc1 ; c1ccc(-c2ccc(N(c3ccc(-c4ccc(-n5c6ccccc6c6c7ccccc7ccc65)cc4)cc3)c3ccc4sc5ccccc5c4c3)cc2)cc1] 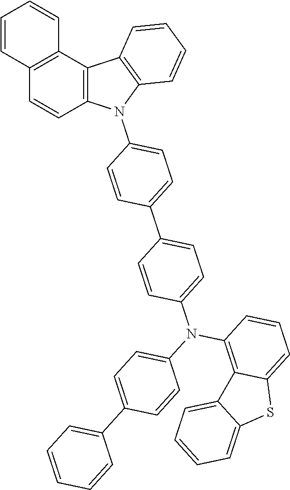 [SMILES: c1ccc(-c2ccc(N(c3ccc(-c4ccc(-n5c6ccccc6c6c7ccccc7ccc65)cc4)cc3)c3cccc4sc5ccccc5c34)cc2)cc1] 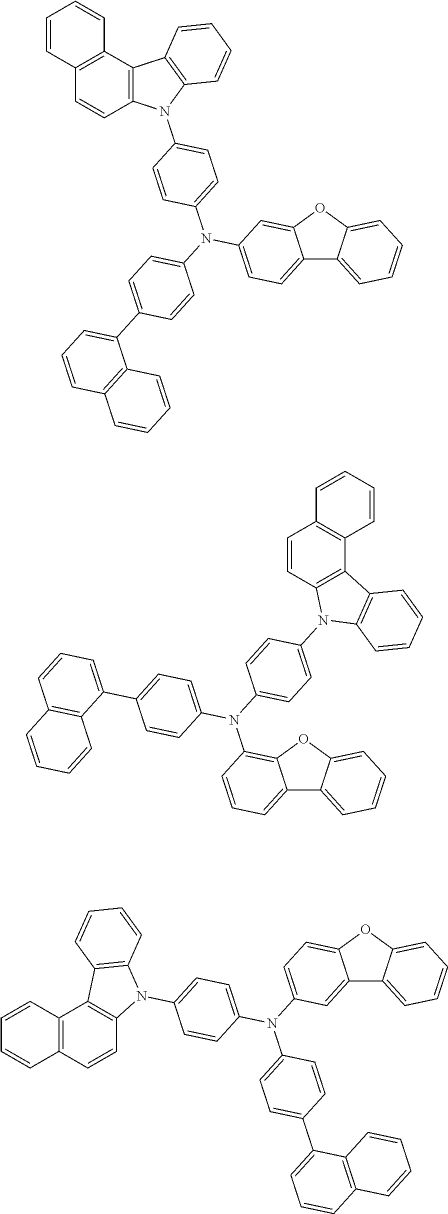 [SMILES: c1ccc2c(-c3ccc(N(c4ccc(-n5c6ccccc6c6c7ccccc7ccc65)cc4)c4ccc5c(c4)oc4ccccc45)cc3)cccc2c1 ; c1ccc2c(-c3ccc(N(c4ccc(-n5c6ccccc6c6c7ccccc7ccc65)cc4)c4cccc5c4oc4ccccc45)cc3)cccc2c1 ; c1ccc2c(-c3ccc(N(c4ccc(-n5c6ccccc6c6c7ccccc7ccc65)cc4)c4ccc5oc6ccccc6c5c4)cc3)cccc2c1] 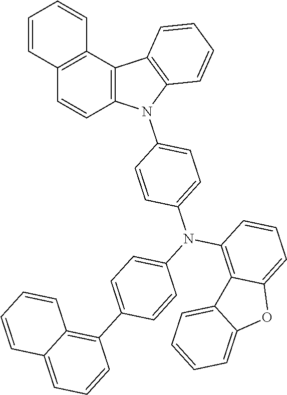 [SMILES: c1ccc2c(-c3ccc(N(c4ccc(-n5c6ccccc6c6c7ccccc7ccc65)cc4)c4cccc5oc6ccccc6c45)cc3)cccc2c1] 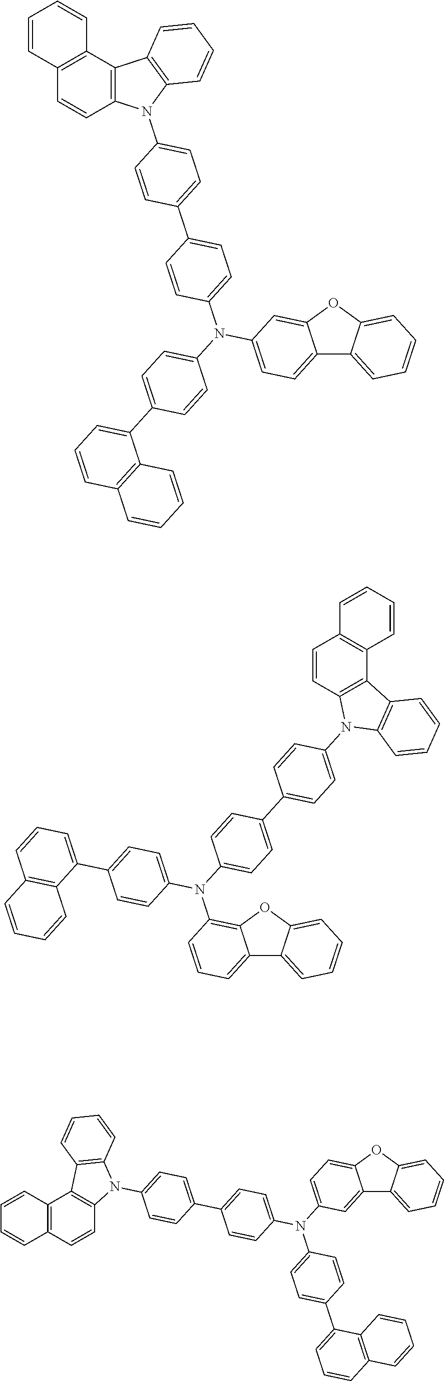 [SMILES: c1ccc2c(-c3ccc(N(c4ccc(-c5ccc(-n6c7ccccc7c7c8ccccc8ccc76)cc5)cc4)c4ccc5c(c4)oc4ccccc45)cc3)cccc2c1 ; c1ccc2c(-c3ccc(N(c4ccc(-c5ccc(-n6c7ccccc7c7c8ccccc8ccc76)cc5)cc4)c4cccc5c4oc4ccccc45)cc3)cccc2c1 ; c1ccc2c(-c3ccc(N(c4ccc(-c5ccc(-n6c7ccccc7c7c8ccccc8ccc76)cc5)cc4)c4ccc5oc6ccccc6c5c4)cc3)cccc2c1] 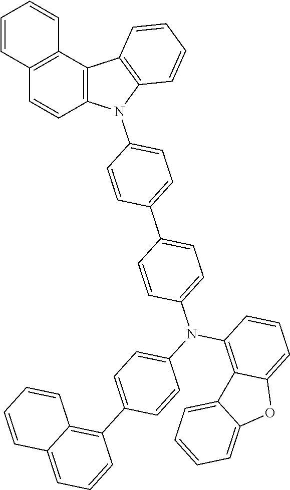 [SMILES: c1ccc2c(-c3ccc(N(c4ccc(-c5ccc(-n6c7ccccc7c7c8ccccc8ccc76)cc5)cc4)c4cccc5oc6ccccc6c45)cc3)cccc2c1] 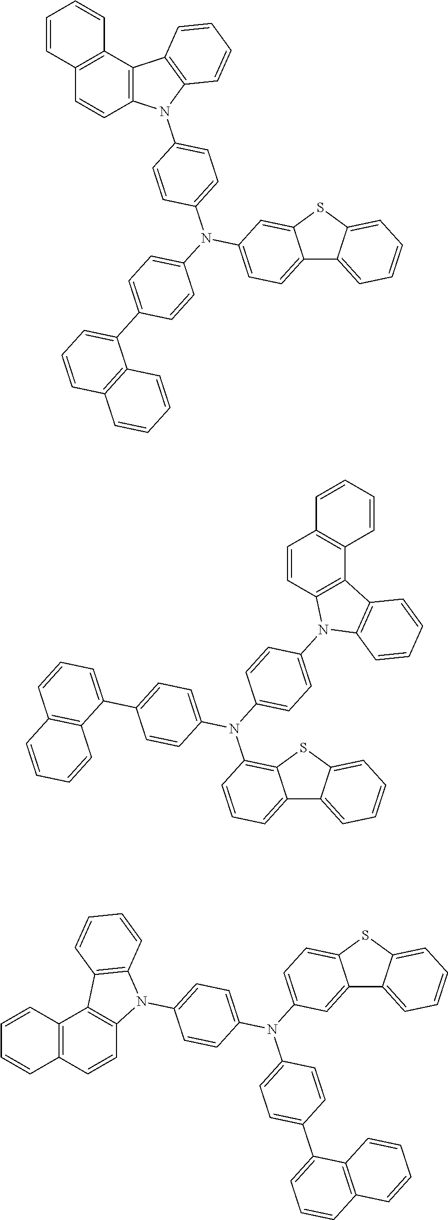 [SMILES: c1ccc2c(-c3ccc(N(c4ccc(-n5c6ccccc6c6c7ccccc7ccc65)cc4)c4ccc5c(c4)sc4ccccc45)cc3)cccc2c1 ; c1ccc2c(-c3ccc(N(c4ccc(-n5c6ccccc6c6c7ccccc7ccc65)cc4)c4cccc5c4sc4ccccc45)cc3)cccc2c1 ; c1ccc2c(-c3ccc(N(c4ccc(-n5c6ccccc6c6c7ccccc7ccc65)cc4)c4ccc5sc6ccccc6c5c4)cc3)cccc2c1] 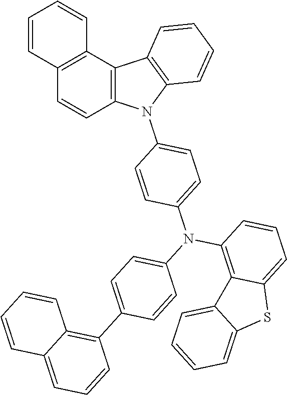 [SMILES: c1ccc2c(-c3ccc(N(c4ccc(-n5c6ccccc6c6c7ccccc7ccc65)cc4)c4cccc5sc6ccccc6c45)cc3)cccc2c1] 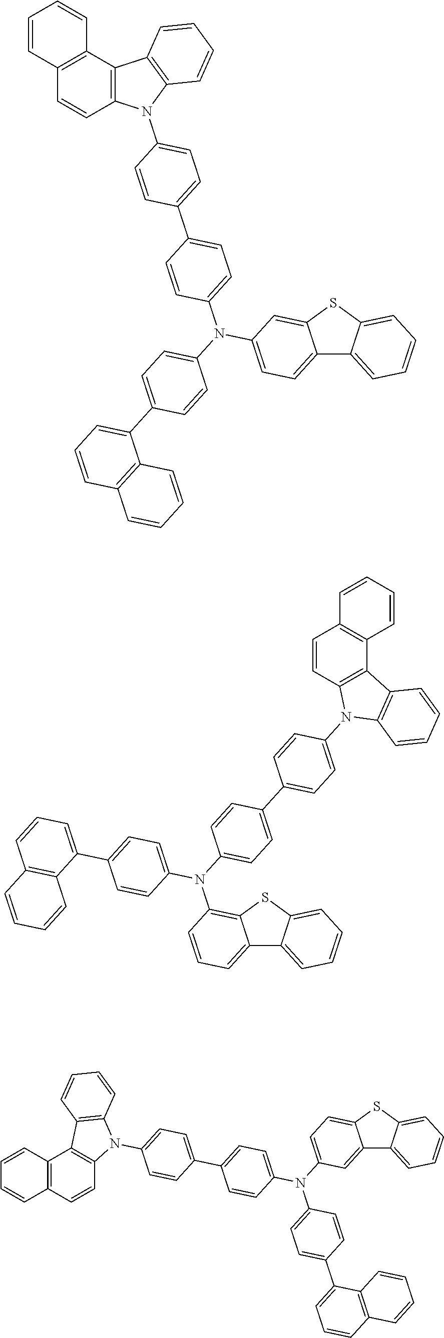 [SMILES: c1ccc2c(-c3ccc(N(c4ccc(-c5ccc(-n6c7ccccc7c7c8ccccc8ccc76)cc5)cc4)c4ccc5c(c4)sc4ccccc45)cc3)cccc2c1 ; c1ccc2c(-c3ccc(N(c4ccc(-c5ccc(-n6c7ccccc7c7c8ccccc8ccc76)cc5)cc4)c4cccc5c4sc4ccccc45)cc3)cccc2c1 ; c1ccc2c(-c3ccc(N(c4ccc(-c5ccc(-n6c7ccccc7c7c8ccccc8ccc76)cc5)cc4)c4ccc5sc6ccccc6c5c4)cc3)cccc2c1] 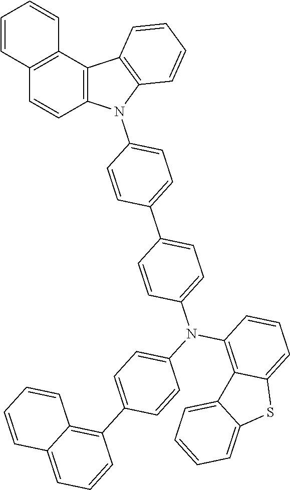 [SMILES: c1ccc2c(-c3ccc(N(c4ccc(-c5ccc(-n6c7ccccc7c7c8ccccc8ccc76)cc5)cc4)c4cccc5sc6ccccc6c45)cc3)cccc2c1] 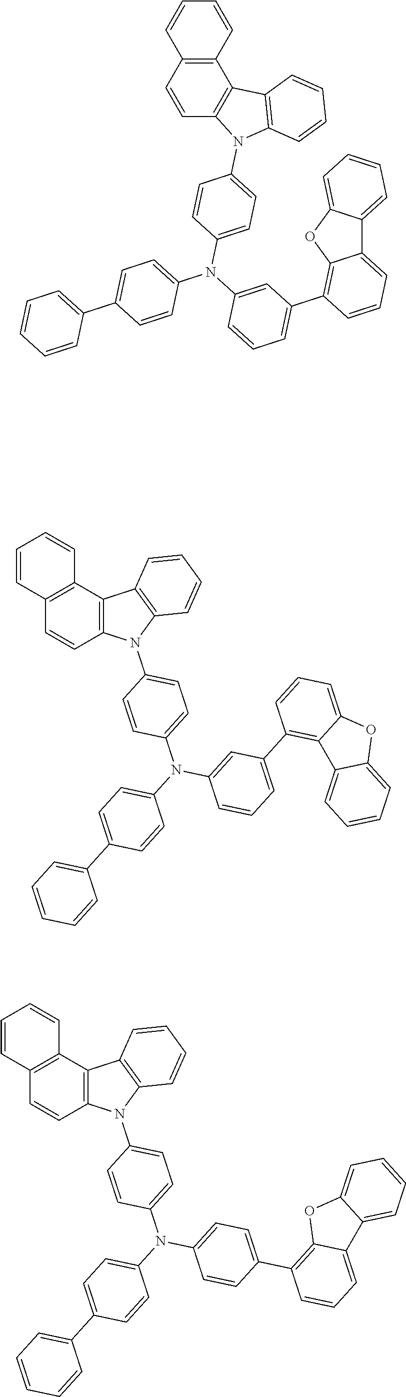 [SMILES: c1ccc(-c2ccc(N(c3ccc(-n4c5ccccc5c5c6ccccc6ccc54)cc3)c3cccc(-c4cccc5c4oc4ccccc45)c3)cc2)cc1 ; c1ccc(-c2ccc(N(c3ccc(-n4c5ccccc5c5c6ccccc6ccc54)cc3)c3cccc(-c4cccc5oc6ccccc6c45)c3)cc2)cc1 ; c1ccc(-c2ccc(N(c3ccc(-c4cccc5c4oc4ccccc45)cc3)c3ccc(-n4c5ccccc5c5c6ccccc6ccc54)cc3)cc2)cc1] 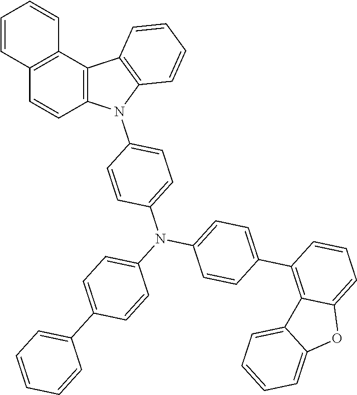 [SMILES: c1ccc(-c2ccc(N(c3ccc(-c4cccc5oc6ccccc6c45)cc3)c3ccc(-n4c5ccccc5c5c6ccccc6ccc54)cc3)cc2)cc1] 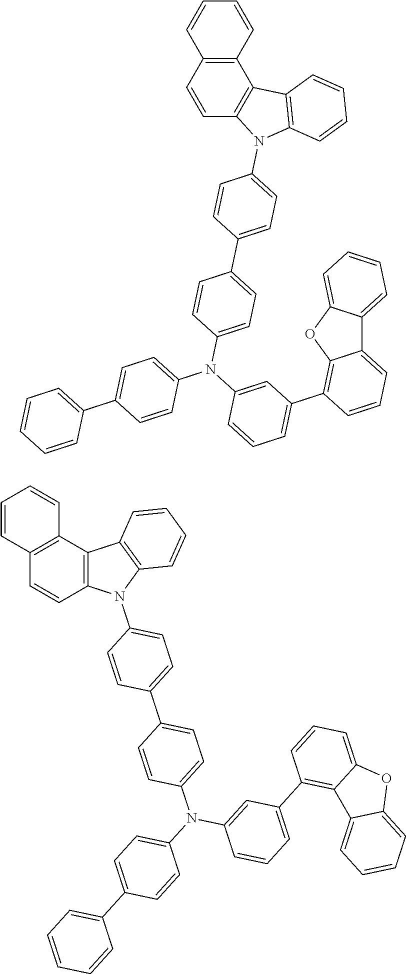 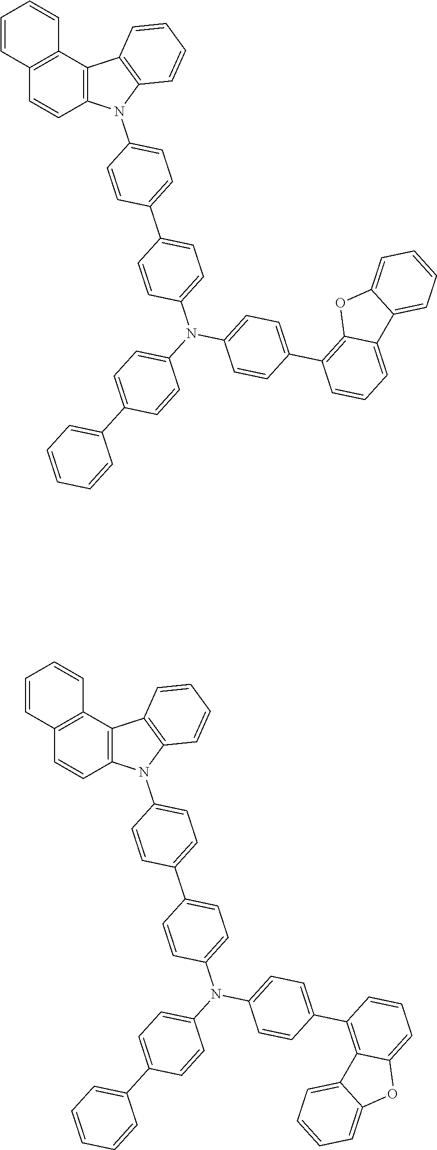 [SMILES: c1ccc(-c2ccc(N(c3ccc(-c4ccc(-n5c6ccccc6c6c7ccccc7ccc65)cc4)cc3)c3ccc(-c4cccc5c4oc4ccccc45)cc3)cc2)cc1 ; c1ccc(-c2ccc(N(c3ccc(-c4ccc(-n5c6ccccc6c6c7ccccc7ccc65)cc4)cc3)c3ccc(-c4cccc5oc6ccccc6c45)cc3)cc2)cc1] 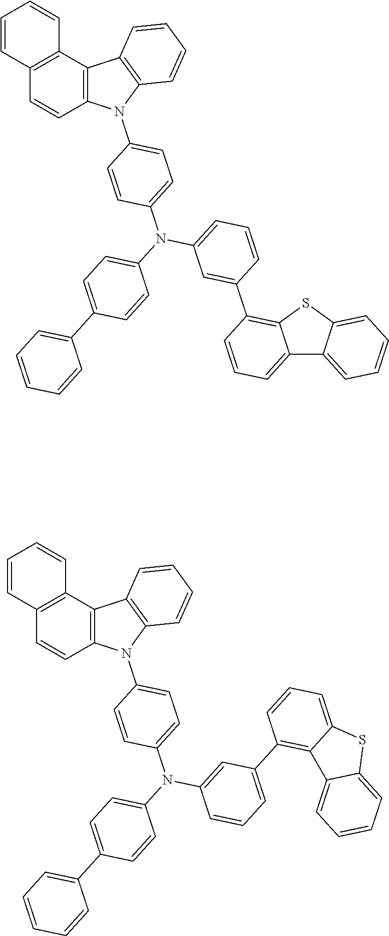 [SMILES: c1ccc(-c2ccc(N(c3ccc(-n4c5ccccc5c5c6ccccc6ccc54)cc3)c3cccc(-c4cccc5c4sc4ccccc45)c3)cc2)cc1 ; c1ccc(-c2ccc(N(c3ccc(-n4c5ccccc5c5c6ccccc6ccc54)cc3)c3cccc(-c4cccc5sc6ccccc6c45)c3)cc2)cc1] 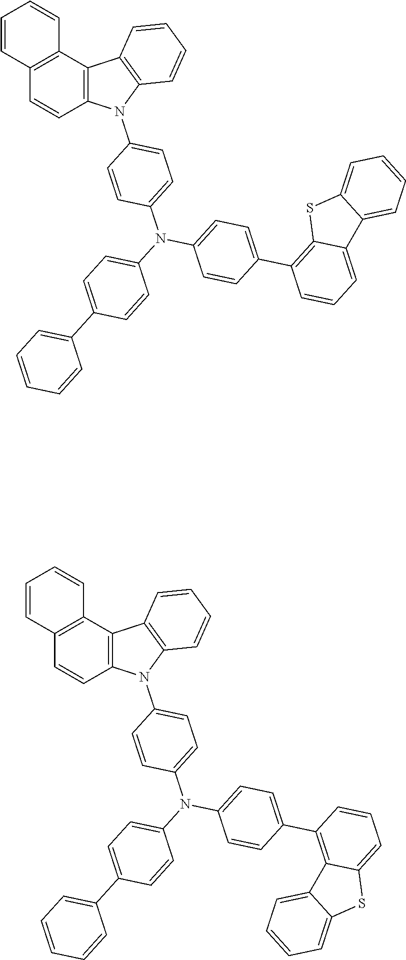 [SMILES: c1ccc(-c2ccc(N(c3ccc(-c4cccc5c4sc4ccccc45)cc3)c3ccc(-n4c5ccccc5c5c6ccccc6ccc54)cc3)cc2)cc1 ; c1ccc(-c2ccc(N(c3ccc(-c4cccc5sc6ccccc6c45)cc3)c3ccc(-n4c5ccccc5c5c6ccccc6ccc54)cc3)cc2)cc1] 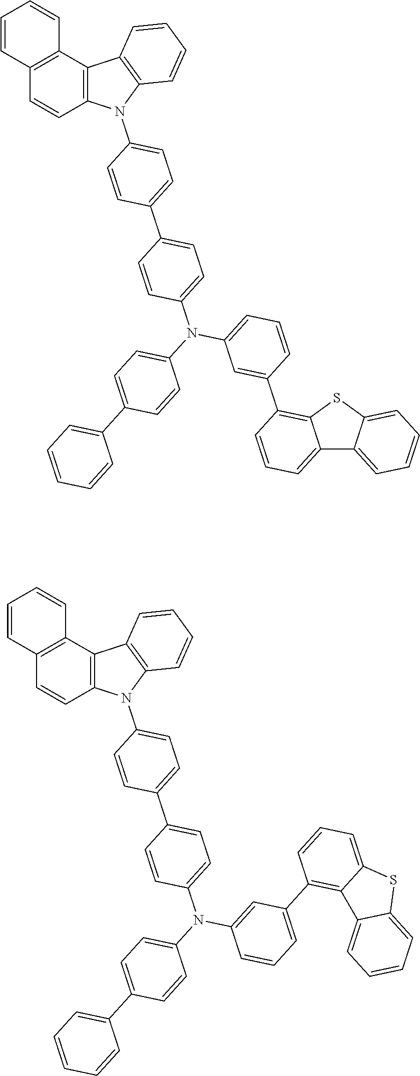 [SMILES: c1ccc(-c2ccc(N(c3ccc(-c4ccc(-n5c6ccccc6c6c7ccccc7ccc65)cc4)cc3)c3cccc(-c4cccc5c4sc4ccccc45)c3)cc2)cc1 ; c1ccc(-c2ccc(N(c3ccc(-c4ccc(-n5c6ccccc6c6c7ccccc7ccc65)cc4)cc3)c3cccc(-c4cccc5sc6ccccc6c45)c3)cc2)cc1] 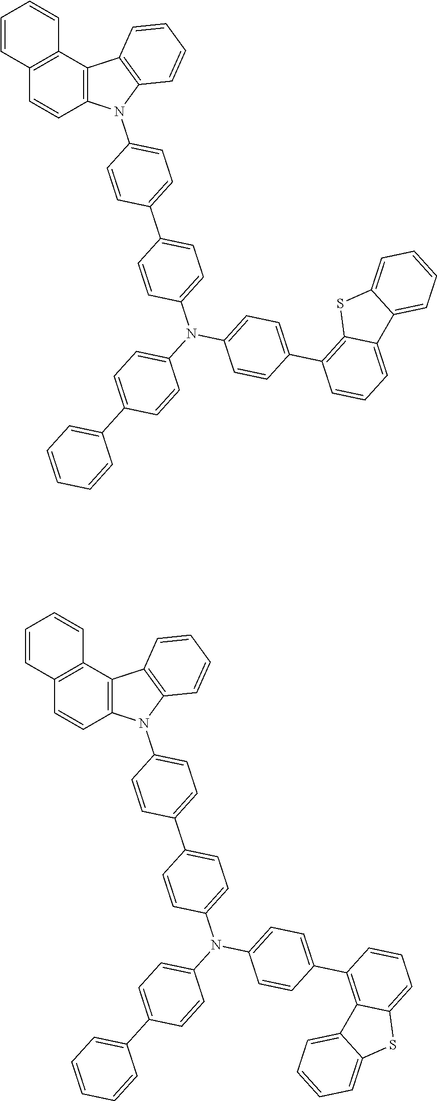 [SMILES: c1ccc(-c2ccc(N(c3ccc(-c4ccc(-n5c6ccccc6c6c7ccccc7ccc65)cc4)cc3)c3ccc(-c4cccc5c4sc4ccccc45)cc3)cc2)cc1 ; c1ccc(-c2ccc(N(c3ccc(-c4ccc(-n5c6ccccc6c6c7ccccc7ccc65)cc4)cc3)c3ccc(-c4cccc5sc6ccccc6c45)cc3)cc2)cc1] 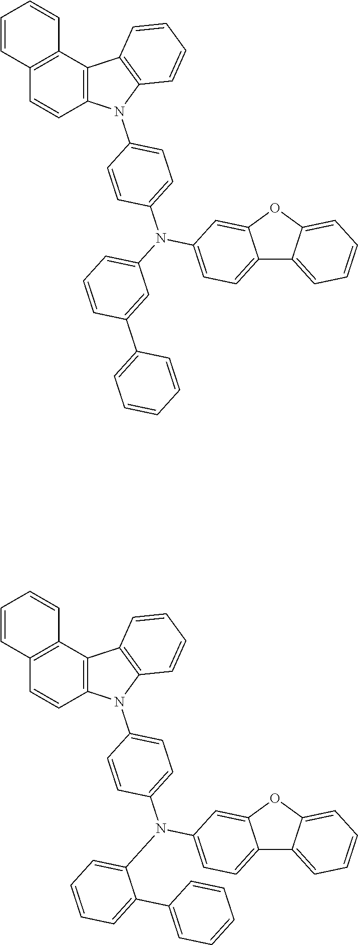 [SMILES: c1ccc(-c2cccc(N(c3ccc(-n4c5ccccc5c5c6ccccc6ccc54)cc3)c3ccc4c(c3)oc3ccccc34)c2)cc1 ; c1ccc(-c2ccccc2N(c2ccc(-n3c4ccccc4c4c5ccccc5ccc43)cc2)c2ccc3c(c2)oc2ccccc23)cc1] 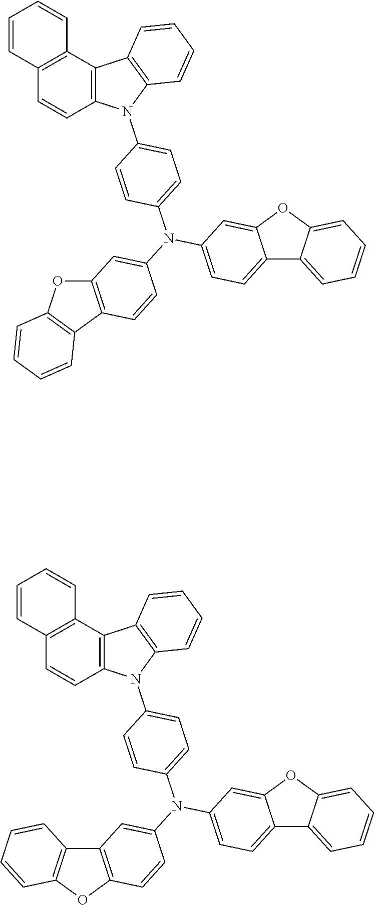 [SMILES: c1ccc2c(c1)ccc1c2c2ccccc2n1-c1ccc(N(c2ccc3c(c2)oc2ccccc23)c2ccc3c(c2)oc2ccccc23)cc1 ; c1ccc2c(c1)ccc1c2c2ccccc2n1-c1ccc(N(c2ccc3c(c2)oc2ccccc23)c2ccc3oc4ccccc4c3c2)cc1] 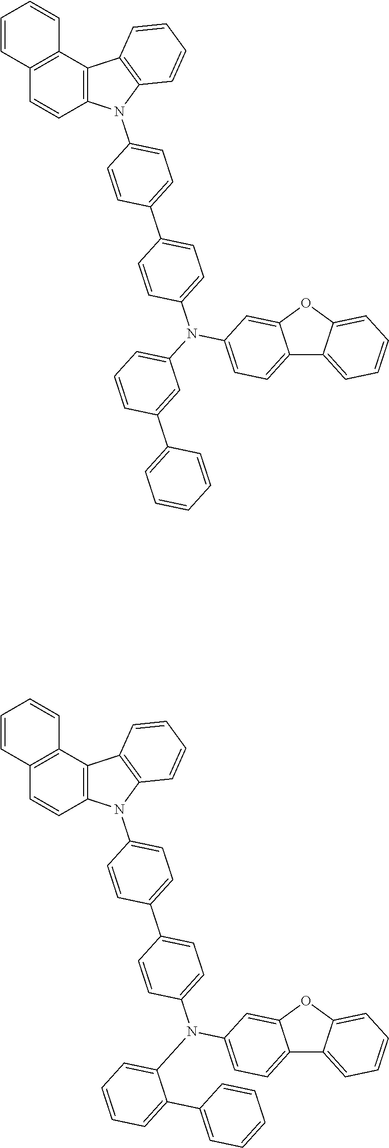 [SMILES: c1ccc(-c2cccc(N(c3ccc(-c4ccc(-n5c6ccccc6c6c7ccccc7ccc65)cc4)cc3)c3ccc4c(c3)oc3ccccc34)c2)cc1 ; c1ccc(-c2ccccc2N(c2ccc(-c3ccc(-n4c5ccccc5c5c6ccccc6ccc54)cc3)cc2)c2ccc3c(c2)oc2ccccc23)cc1] 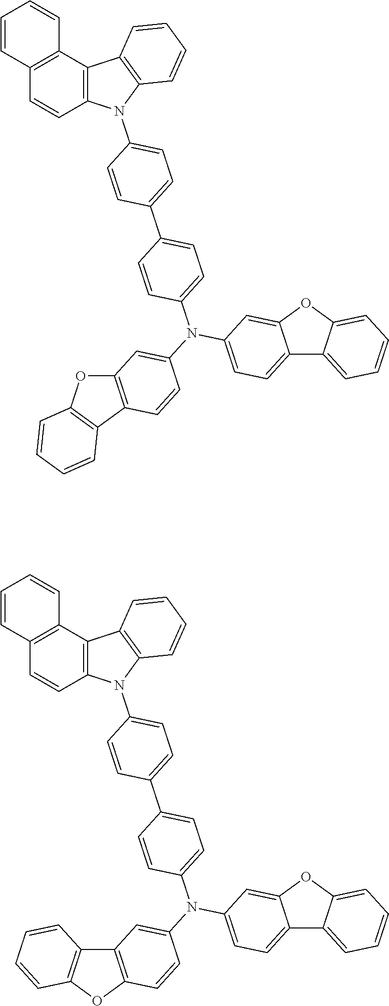 [SMILES: c1ccc2c(c1)ccc1c2c2ccccc2n1-c1ccc(-c2ccc(N(c3ccc4c(c3)oc3ccccc34)c3ccc4c(c3)oc3ccccc34)cc2)cc1 ; c1ccc2c(c1)ccc1c2c2ccccc2n1-c1ccc(-c2ccc(N(c3ccc4c(c3)oc3ccccc34)c3ccc4oc5ccccc5c4c3)cc2)cc1] 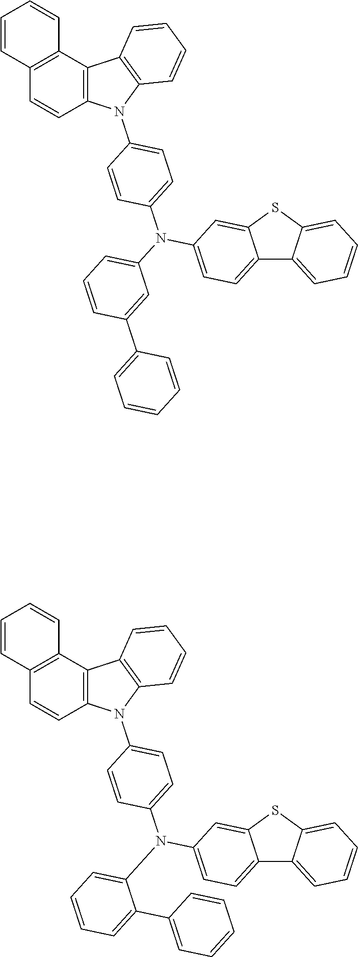 [SMILES: c1ccc(-c2cccc(N(c3ccc(-n4c5ccccc5c5c6ccccc6ccc54)cc3)c3ccc4c(c3)sc3ccccc34)c2)cc1 ; c1ccc(-c2ccccc2N(c2ccc(-n3c4ccccc4c4c5ccccc5ccc43)cc2)c2ccc3c(c2)sc2ccccc23)cc1] 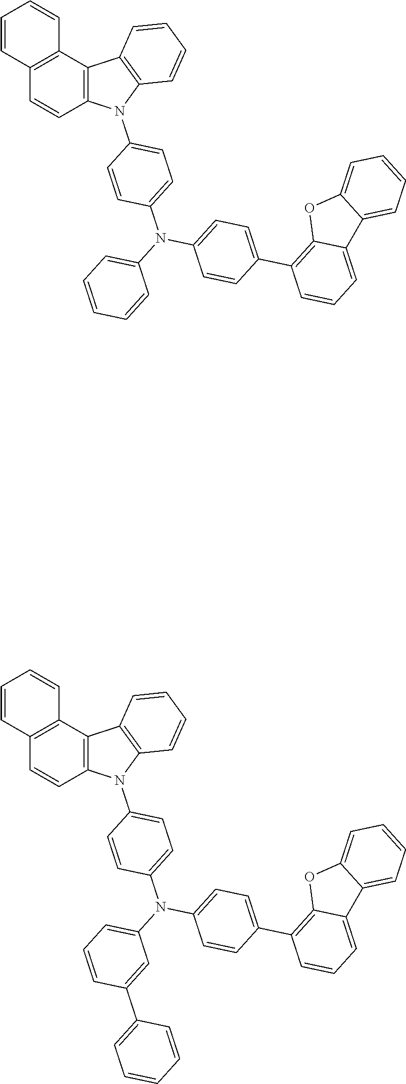 [SMILES: c1ccc(N(c2ccc(-c3cccc4c3oc3ccccc34)cc2)c2ccc(-n3c4ccccc4c4c5ccccc5ccc43)cc2)cc1 ; c1ccc(-c2cccc(N(c3ccc(-c4cccc5c4oc4ccccc45)cc3)c3ccc(-n4c5ccccc5c5c6ccccc6ccc54)cc3)c2)cc1] 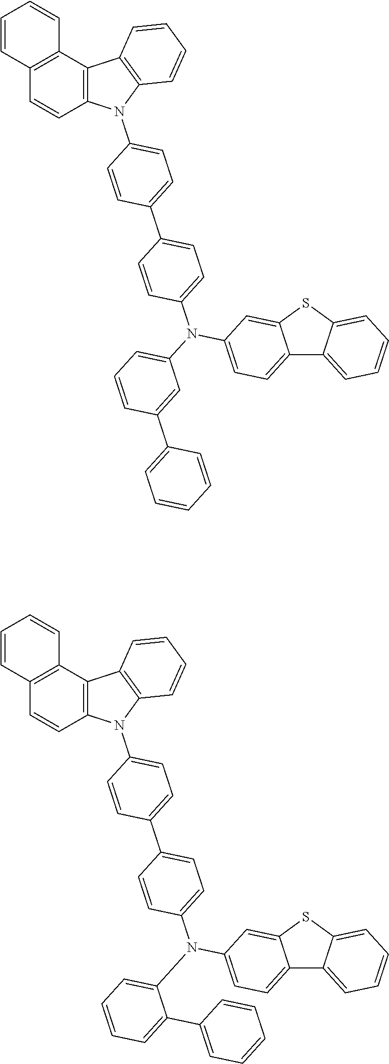 [SMILES: c1ccc(-c2cccc(N(c3ccc(-c4ccc(-n5c6ccccc6c6c7ccccc7ccc65)cc4)cc3)c3ccc4c(c3)sc3ccccc34)c2)cc1 ; c1ccc(-c2ccccc2N(c2ccc(-c3ccc(-n4c5ccccc5c5c6ccccc6ccc54)cc3)cc2)c2ccc3c(c2)sc2ccccc23)cc1] 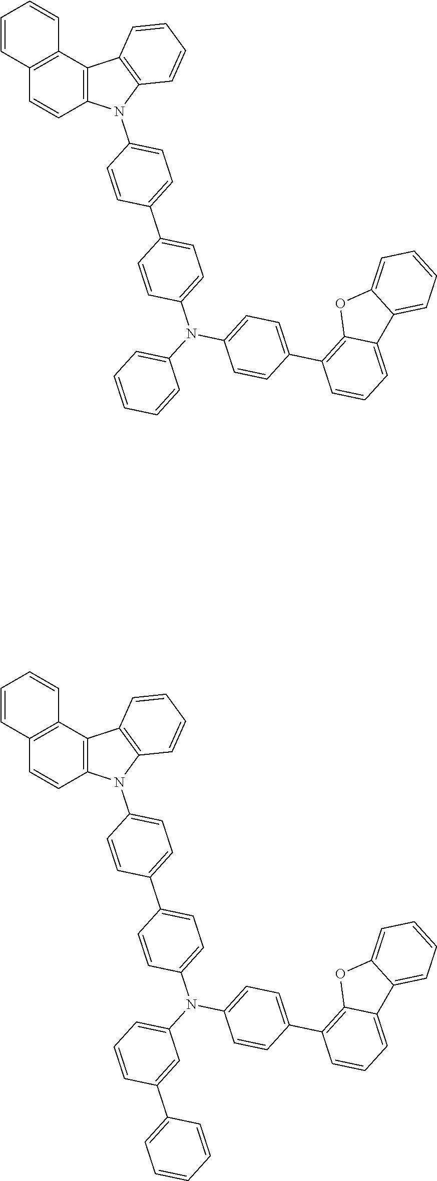 [SMILES: c1ccc(N(c2ccc(-c3ccc(-n4c5ccccc5c5c6ccccc6ccc54)cc3)cc2)c2ccc(-c3cccc4c3oc3ccccc34)cc2)cc1 ; c1ccc(-c2cccc(N(c3ccc(-c4ccc(-n5c6ccccc6c6c7ccccc7ccc65)cc4)cc3)c3ccc(-c4cccc5c4oc4ccccc45)cc3)c2)cc1] 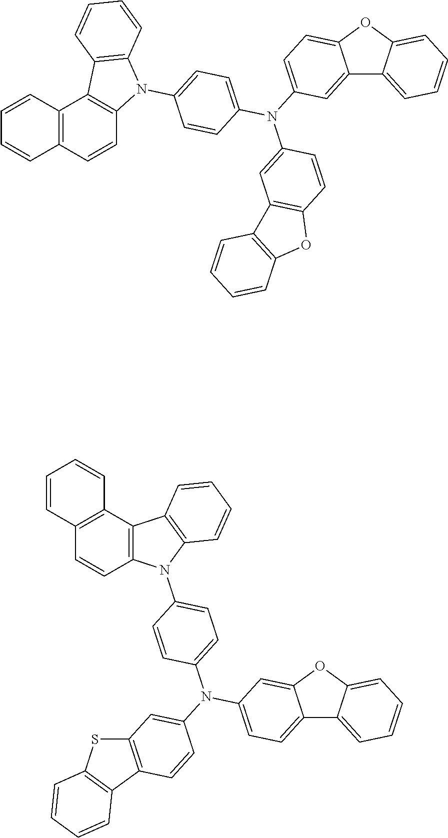 [SMILES: c1ccc2c(c1)ccc1c2c2ccccc2n1-c1ccc(N(c2ccc3oc4ccccc4c3c2)c2ccc3oc4ccccc4c3c2)cc1 ; c1ccc2c(c1)ccc1c2c2ccccc2n1-c1ccc(N(c2ccc3c(c2)oc2ccccc23)c2ccc3c(c2)sc2ccccc23)cc1] 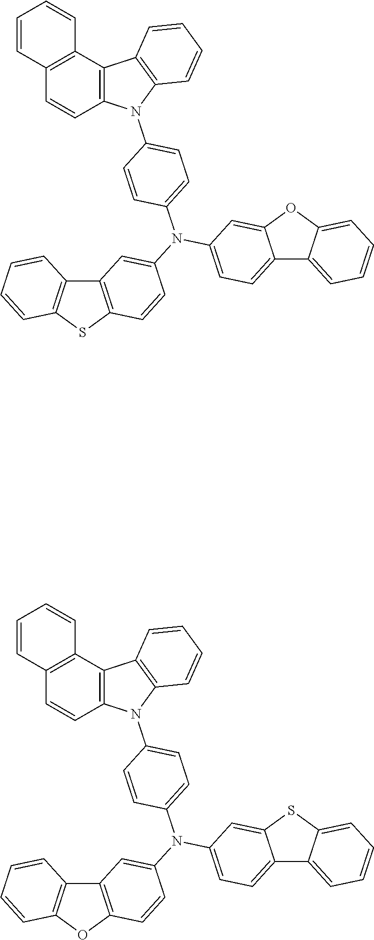 [SMILES: c1ccc2c(c1)ccc1c2c2ccccc2n1-c1ccc(N(c2ccc3c(c2)oc2ccccc23)c2ccc3sc4ccccc4c3c2)cc1 ; c1ccc2c(c1)ccc1c2c2ccccc2n1-c1ccc(N(c2ccc3c(c2)sc2ccccc23)c2ccc3oc4ccccc4c3c2)cc1] 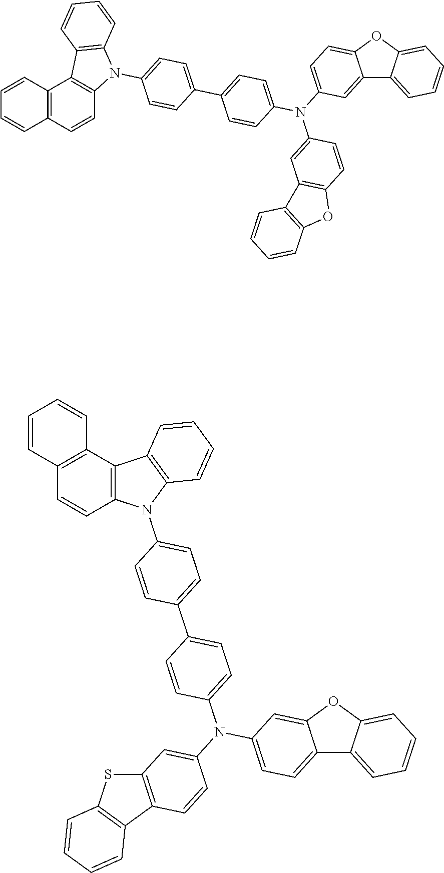 [SMILES: c1ccc2c(c1)ccc1c2c2ccccc2n1-c1ccc(-c2ccc(N(c3ccc4oc5ccccc5c4c3)c3ccc4oc5ccccc5c4c3)cc2)cc1 ; c1ccc2c(c1)ccc1c2c2ccccc2n1-c1ccc(-c2ccc(N(c3ccc4c(c3)oc3ccccc34)c3ccc4c(c3)sc3ccccc34)cc2)cc1] 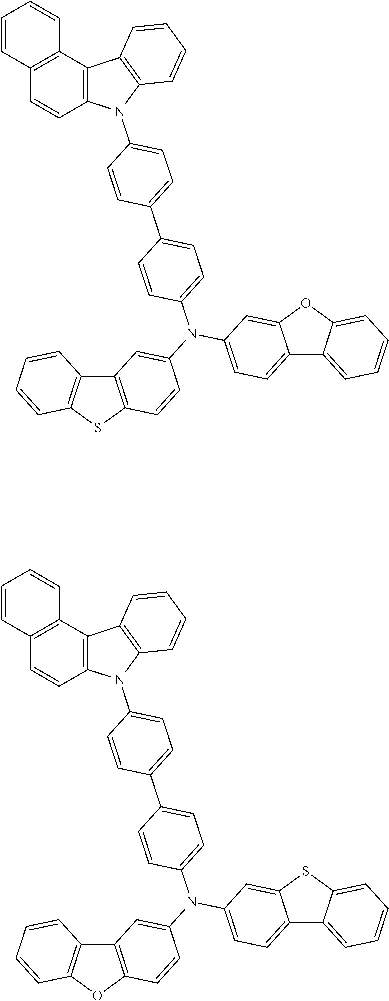 [SMILES: c1ccc2c(c1)ccc1c2c2ccccc2n1-c1ccc(-c2ccc(N(c3ccc4c(c3)oc3ccccc34)c3ccc4sc5ccccc5c4c3)cc2)cc1 ; c1ccc2c(c1)ccc1c2c2ccccc2n1-c1ccc(-c2ccc(N(c3ccc4c(c3)sc3ccccc34)c3ccc4oc5ccccc5c4c3)cc2)cc1] 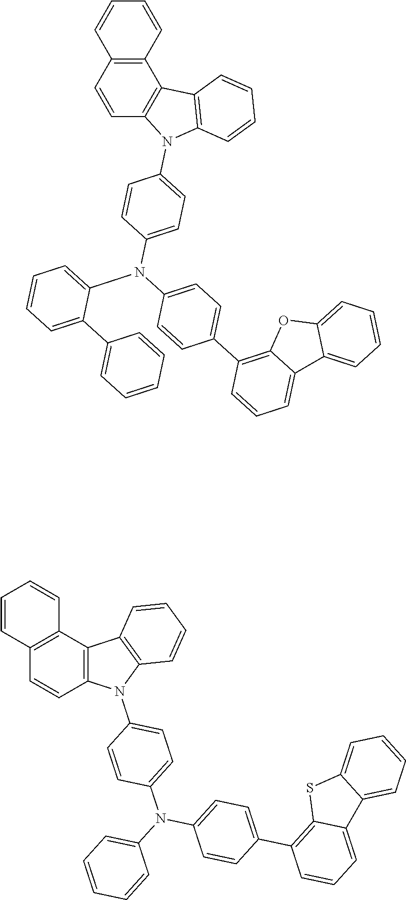 [SMILES: c1ccc(-c2ccccc2N(c2ccc(-c3cccc4c3oc3ccccc34)cc2)c2ccc(-n3c4ccccc4c4c5ccccc5ccc43)cc2)cc1 ; c1ccc(N(c2ccc(-c3cccc4c3sc3ccccc34)cc2)c2ccc(-n3c4ccccc4c4c5ccccc5ccc43)cc2)cc1] 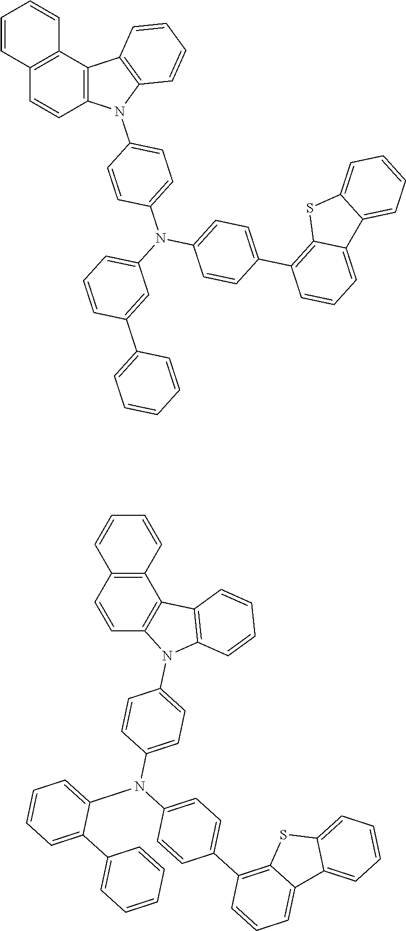 [SMILES: c1ccc(-c2cccc(N(c3ccc(-c4cccc5c4sc4ccccc45)cc3)c3ccc(-n4c5ccccc5c5c6ccccc6ccc54)cc3)c2)cc1 ; c1ccc(-c2ccccc2N(c2ccc(-c3cccc4c3sc3ccccc34)cc2)c2ccc(-n3c4ccccc4c4c5ccccc5ccc43)cc2)cc1] 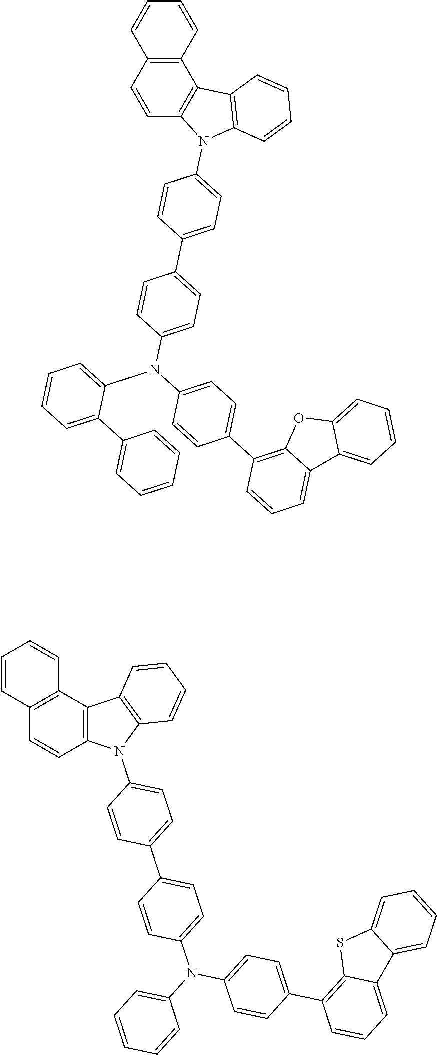 [SMILES: c1ccc(-c2ccccc2N(c2ccc(-c3ccc(-n4c5ccccc5c5c6ccccc6ccc54)cc3)cc2)c2ccc(-c3cccc4c3oc3ccccc34)cc2)cc1 ; c1ccc(N(c2ccc(-c3ccc(-n4c5ccccc5c5c6ccccc6ccc54)cc3)cc2)c2ccc(-c3cccc4c3sc3ccccc34)cc2)cc1] 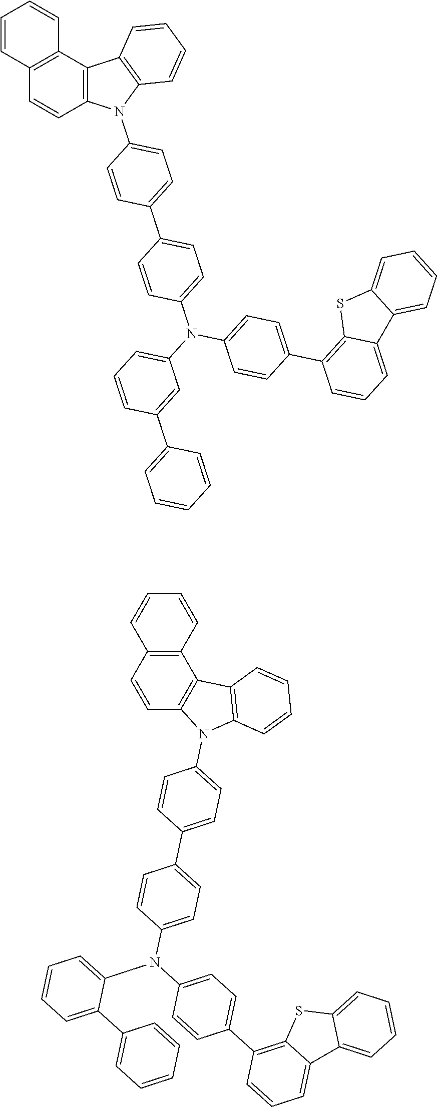 [SMILES: c1ccc(-c2cccc(N(c3ccc(-c4ccc(-n5c6ccccc6c6c7ccccc7ccc65)cc4)cc3)c3ccc(-c4cccc5c4sc4ccccc45)cc3)c2)cc1 ; c1ccc(-c2ccccc2N(c2ccc(-c3ccc(-n4c5ccccc5c5c6ccccc6ccc54)cc3)cc2)c2ccc(-c3cccc4c3sc3ccccc34)cc2)cc1] 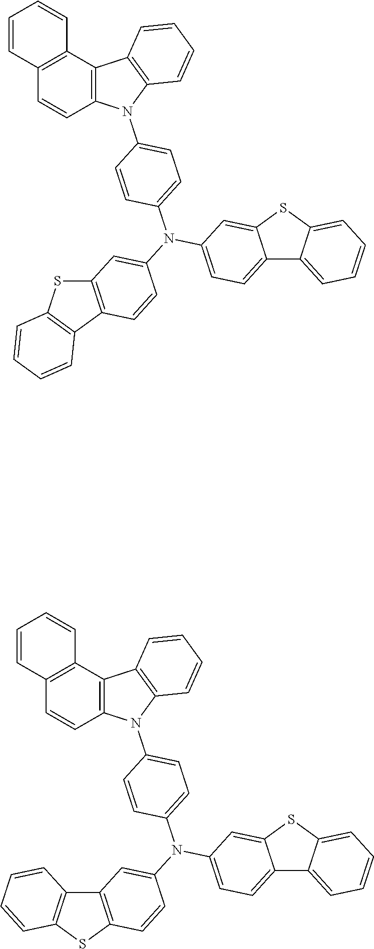 [SMILES: c1ccc2c(c1)ccc1c2c2ccccc2n1-c1ccc(N(c2ccc3c(c2)sc2ccccc23)c2ccc3c(c2)sc2ccccc23)cc1 ; c1ccc2c(c1)ccc1c2c2ccccc2n1-c1ccc(N(c2ccc3c(c2)sc2ccccc23)c2ccc3sc4ccccc4c3c2)cc1] 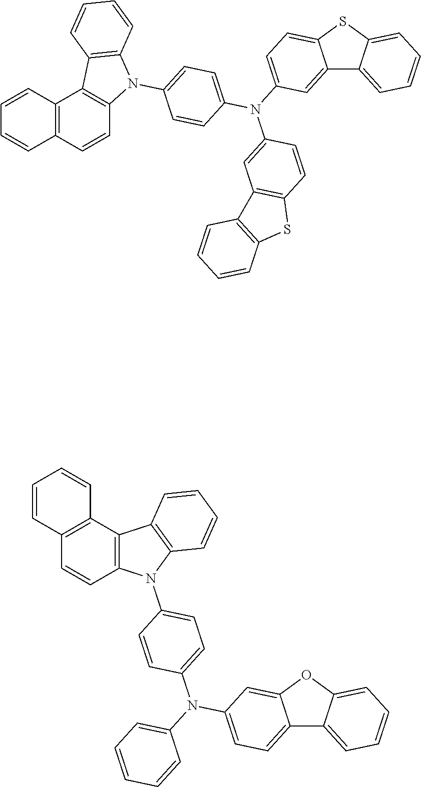 [SMILES: c1ccc2c(c1)ccc1c2c2ccccc2n1-c1ccc(N(c2ccc3sc4ccccc4c3c2)c2ccc3sc4ccccc4c3c2)cc1 ; c1ccc(N(c2ccc(-n3c4ccccc4c4c5ccccc5ccc43)cc2)c2ccc3c(c2)oc2ccccc23)cc1] 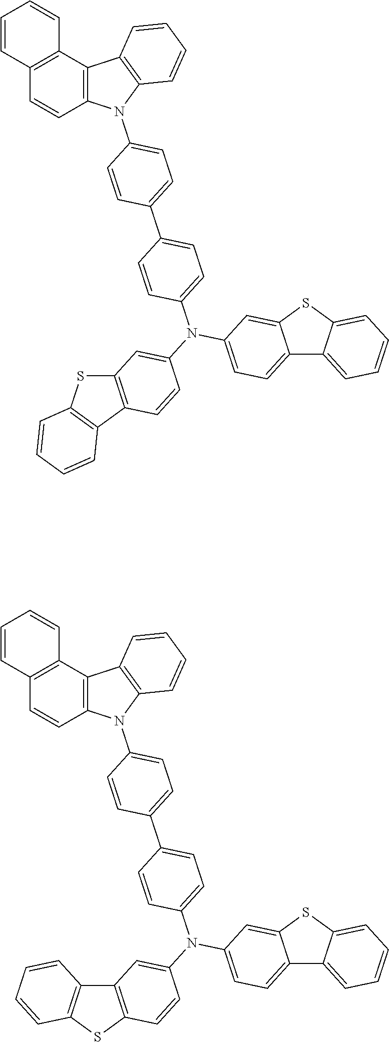 [SMILES: c1ccc2c(c1)ccc1c2c2ccccc2n1-c1ccc(-c2ccc(N(c3ccc4c(c3)sc3ccccc34)c3ccc4c(c3)sc3ccccc34)cc2)cc1 ; c1ccc2c(c1)ccc1c2c2ccccc2n1-c1ccc(-c2ccc(N(c3ccc4c(c3)sc3ccccc34)c3ccc4sc5ccccc5c4c3)cc2)cc1] 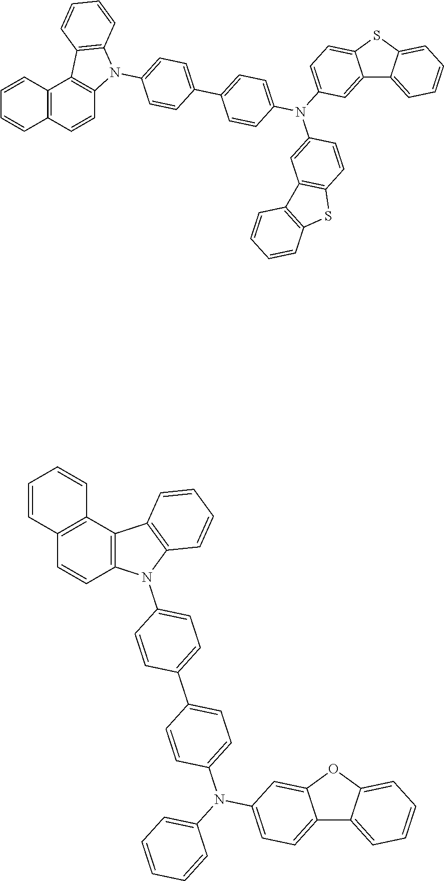 [SMILES: c1ccc2c(c1)ccc1c2c2ccccc2n1-c1ccc(-c2ccc(N(c3ccc4sc5ccccc5c4c3)c3ccc4sc5ccccc5c4c3)cc2)cc1 ; c1ccc(N(c2ccc(-c3ccc(-n4c5ccccc5c5c6ccccc6ccc54)cc3)cc2)c2ccc3c(c2)oc2ccccc23)cc1] 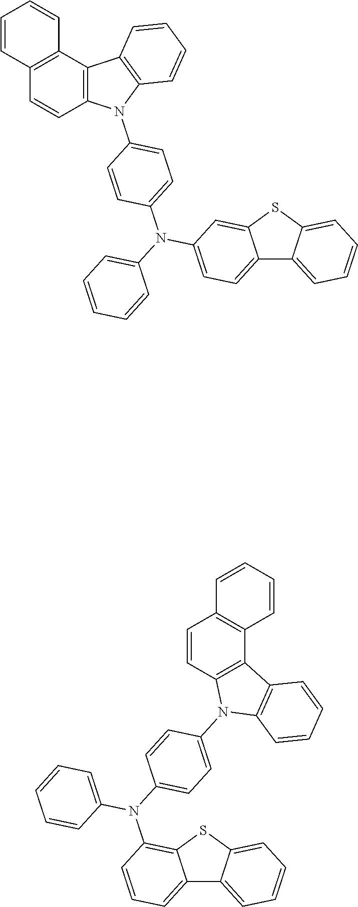 [SMILES: c1ccc(N(c2ccc(-n3c4ccccc4c4c5ccccc5ccc43)cc2)c2ccc3c(c2)sc2ccccc23)cc1 ; c1ccc(N(c2ccc(-n3c4ccccc4c4c5ccccc5ccc43)cc2)c2cccc3c2sc2ccccc23)cc1] 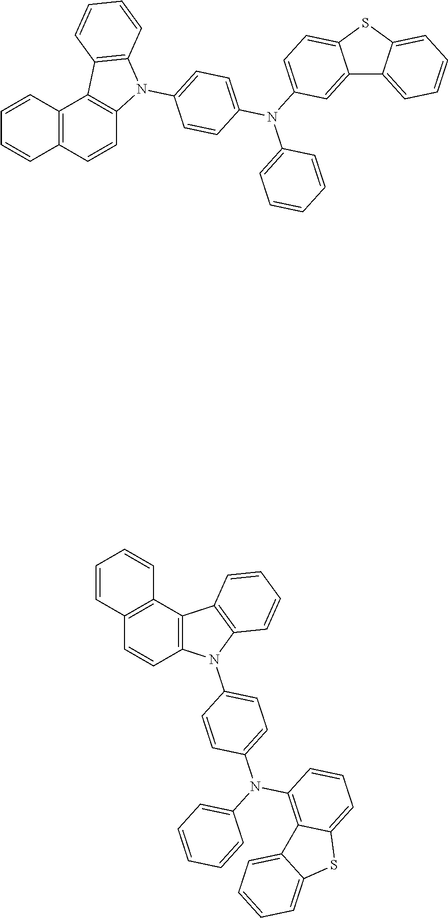 [SMILES: c1ccc(N(c2ccc(-n3c4ccccc4c4c5ccccc5ccc43)cc2)c2ccc3sc4ccccc4c3c2)cc1 ; c1ccc(N(c2ccc(-n3c4ccccc4c4c5ccccc5ccc43)cc2)c2cccc3sc4ccccc4c23)cc1] 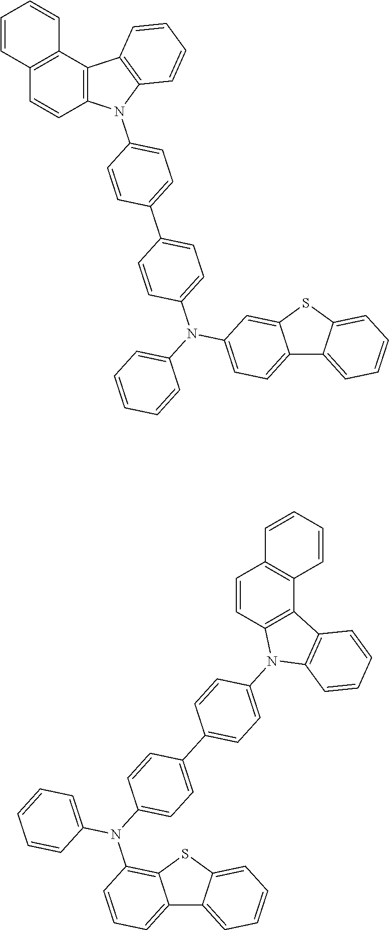 [SMILES: c1ccc(N(c2ccc(-c3ccc(-n4c5ccccc5c5c6ccccc6ccc54)cc3)cc2)c2ccc3c(c2)sc2ccccc23)cc1 ; c1ccc(N(c2ccc(-c3ccc(-n4c5ccccc5c5c6ccccc6ccc54)cc3)cc2)c2cccc3c2sc2ccccc23)cc1] 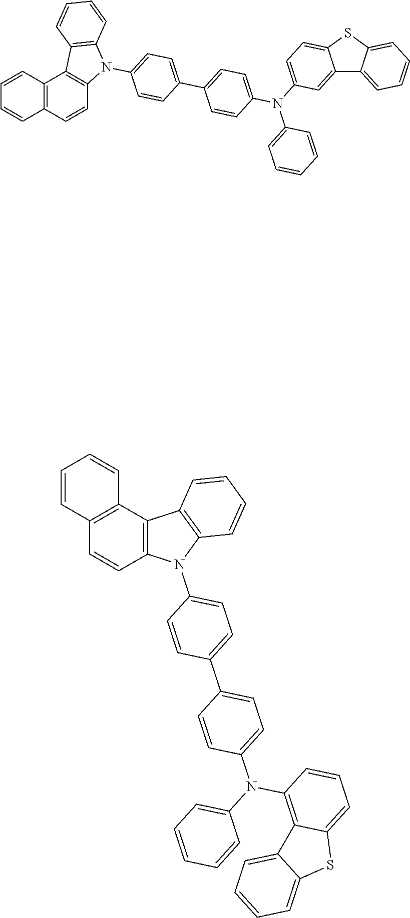 [SMILES: c1ccc(N(c2ccc(-c3ccc(-n4c5ccccc5c5c6ccccc6ccc54)cc3)cc2)c2ccc3sc4ccccc4c3c2)cc1 ; c1ccc(N(c2ccc(-c3ccc(-n4c5ccccc5c5c6ccccc6ccc54)cc3)cc2)c2cccc3sc4ccccc4c23)cc1] 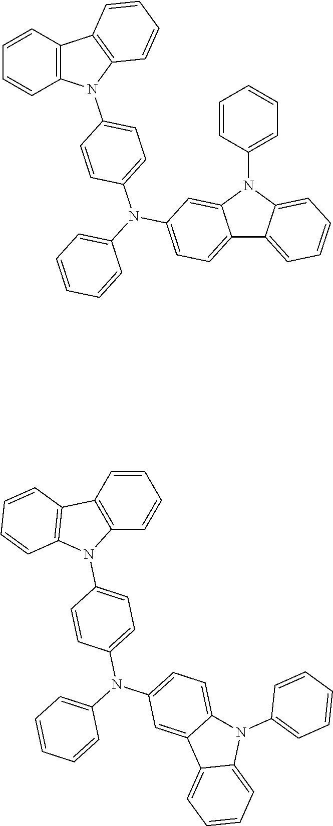 [SMILES: c1ccc(N(c2ccc(-n3c4ccccc4c4ccccc43)cc2)c2ccc3c4ccccc4n(-c4ccccc4)c3c2)cc1 ; c1ccc(N(c2ccc(-n3c4ccccc4c4ccccc43)cc2)c2ccc3c(c2)c2ccccc2n3-c2ccccc2)cc1] 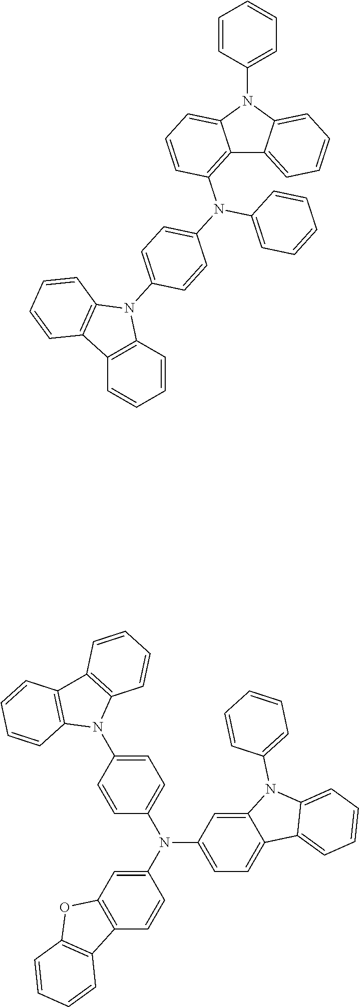 [SMILES: c1ccc(N(c2ccc(-n3c4ccccc4c4ccccc43)cc2)c2cccc3c2c2ccccc2n3-c2ccccc2)cc1 ; c1ccc(-n2c3ccccc3c3ccc(N(c4ccc(-n5c6ccccc6c6ccccc65)cc4)c4ccc5c(c4)oc4ccccc45)cc32)cc1] 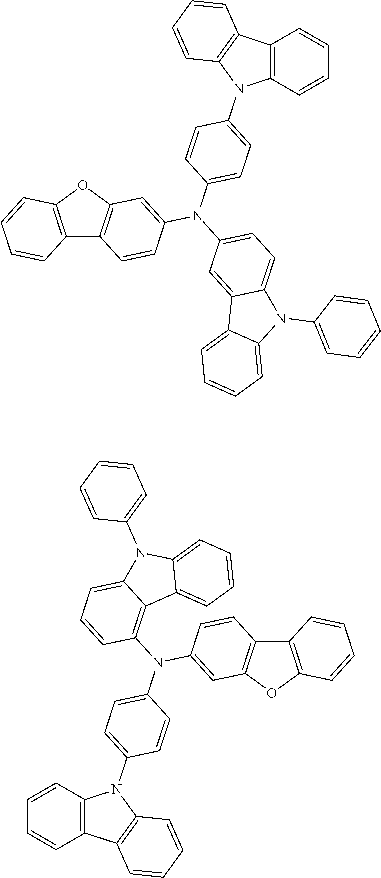 [SMILES: c1ccc(-n2c3ccccc3c3cc(N(c4ccc(-n5c6ccccc6c6ccccc65)cc4)c4ccc5c(c4)oc4ccccc45)ccc32)cc1 ; c1ccc(-n2c3ccccc3c3c(N(c4ccc(-n5c6ccccc6c6ccccc65)cc4)c4ccc5c(c4)oc4ccccc45)cccc32)cc1] 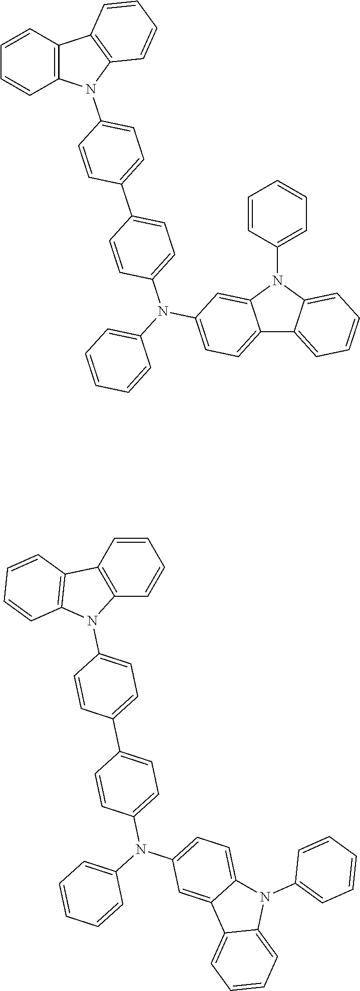 [SMILES: c1ccc(N(c2ccc(-c3ccc(-n4c5ccccc5c5ccccc54)cc3)cc2)c2ccc3c4ccccc4n(-c4ccccc4)c3c2)cc1 ; c1ccc(N(c2ccc(-c3ccc(-n4c5ccccc5c5ccccc54)cc3)cc2)c2ccc3c(c2)c2ccccc2n3-c2ccccc2)cc1] 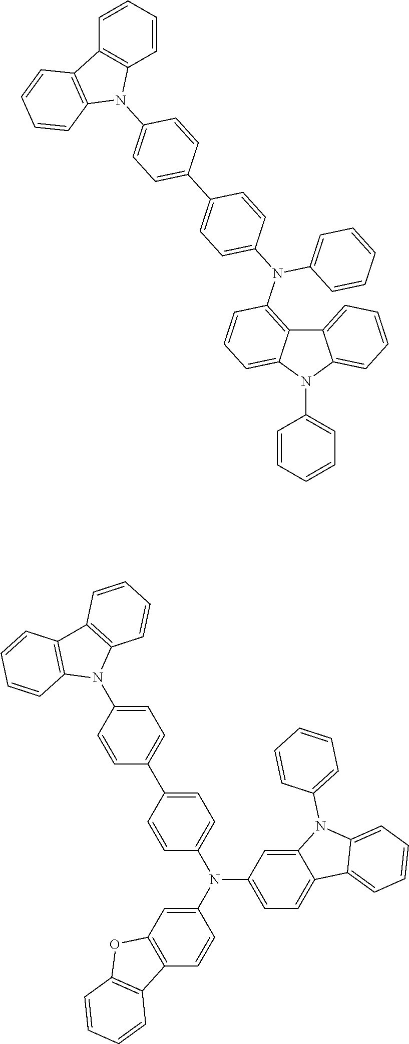 [SMILES: c1ccc(N(c2ccc(-c3ccc(-n4c5ccccc5c5ccccc54)cc3)cc2)c2cccc3c2c2ccccc2n3-c2ccccc2)cc1 ; c1ccc(-n2c3ccccc3c3ccc(N(c4ccc(-c5ccc(-n6c7ccccc7c7ccccc76)cc5)cc4)c4ccc5c(c4)oc4ccccc45)cc32)cc1] 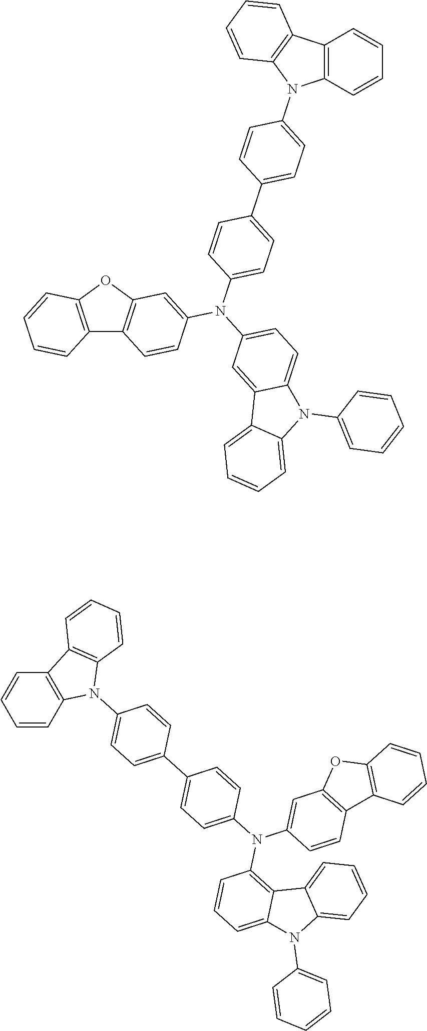 [SMILES: c1ccc(-n2c3ccccc3c3cc(N(c4ccc(-c5ccc(-n6c7ccccc7c7ccccc76)cc5)cc4)c4ccc5c(c4)oc4ccccc45)ccc32)cc1 ; c1ccc(-n2c3ccccc3c3c(N(c4ccc(-c5ccc(-n6c7ccccc7c7ccccc76)cc5)cc4)c4ccc5c(c4)oc4ccccc45)cccc32)cc1] 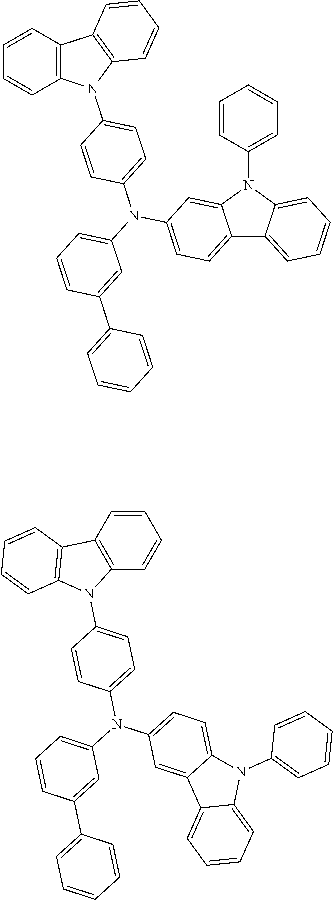 [SMILES: c1ccc(-c2cccc(N(c3ccc(-n4c5ccccc5c5ccccc54)cc3)c3ccc4c5ccccc5n(-c5ccccc5)c4c3)c2)cc1 ; c1ccc(-c2cccc(N(c3ccc(-n4c5ccccc5c5ccccc54)cc3)c3ccc4c(c3)c3ccccc3n4-c3ccccc3)c2)cc1] 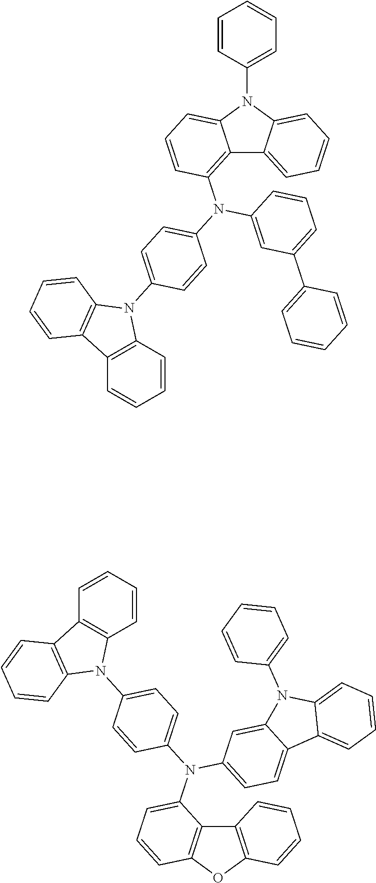 [SMILES: c1ccc(-c2cccc(N(c3ccc(-n4c5ccccc5c5ccccc54)cc3)c3cccc4c3c3ccccc3n4-c3ccccc3)c2)cc1 ; c1ccc(-n2c3ccccc3c3ccc(N(c4ccc(-n5c6ccccc6c6ccccc65)cc4)c4cccc5oc6ccccc6c45)cc32)cc1] 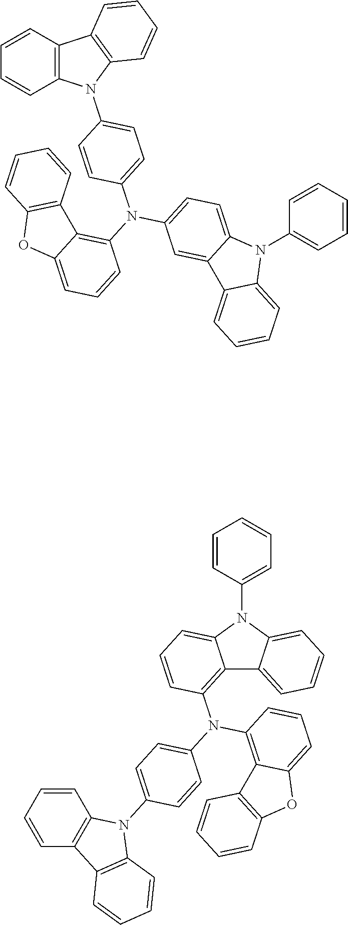 [SMILES: c1ccc(-n2c3ccccc3c3cc(N(c4ccc(-n5c6ccccc6c6ccccc65)cc4)c4cccc5oc6ccccc6c45)ccc32)cc1 ; c1ccc(-n2c3ccccc3c3c(N(c4ccc(-n5c6ccccc6c6ccccc65)cc4)c4cccc5oc6ccccc6c45)cccc32)cc1] 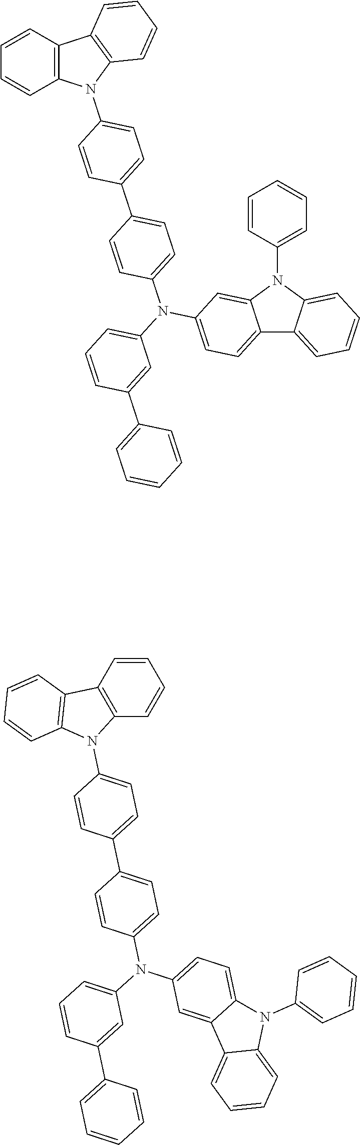 [SMILES: c1ccc(-c2cccc(N(c3ccc(-c4ccc(-n5c6ccccc6c6ccccc65)cc4)cc3)c3ccc4c5ccccc5n(-c5ccccc5)c4c3)c2)cc1 ; c1ccc(-c2cccc(N(c3ccc(-c4ccc(-n5c6ccccc6c6ccccc65)cc4)cc3)c3ccc4c(c3)c3ccccc3n4-c3ccccc3)c2)cc1] 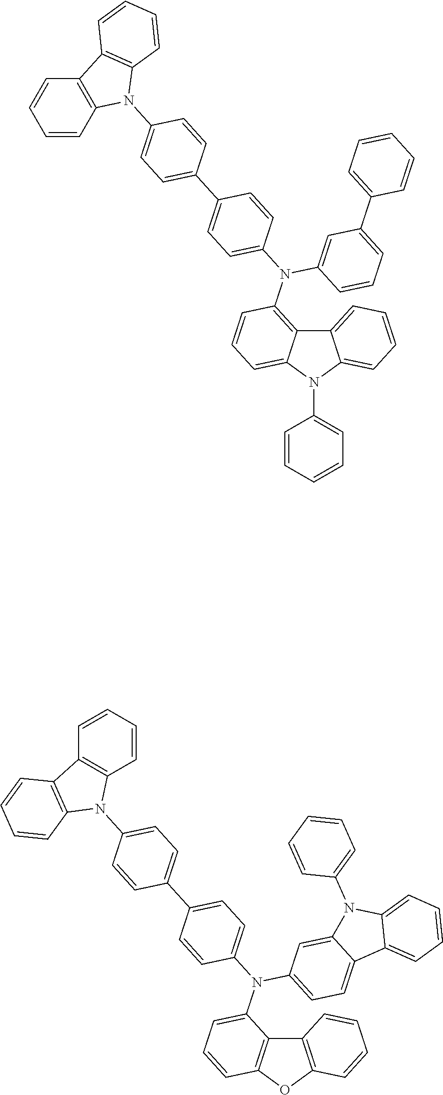 [SMILES: c1ccc(-c2cccc(N(c3ccc(-c4ccc(-n5c6ccccc6c6ccccc65)cc4)cc3)c3cccc4c3c3ccccc3n4-c3ccccc3)c2)cc1 ; c1ccc(-n2c3ccccc3c3ccc(N(c4ccc(-c5ccc(-n6c7ccccc7c7ccccc76)cc5)cc4)c4cccc5oc6ccccc6c45)cc32)cc1] 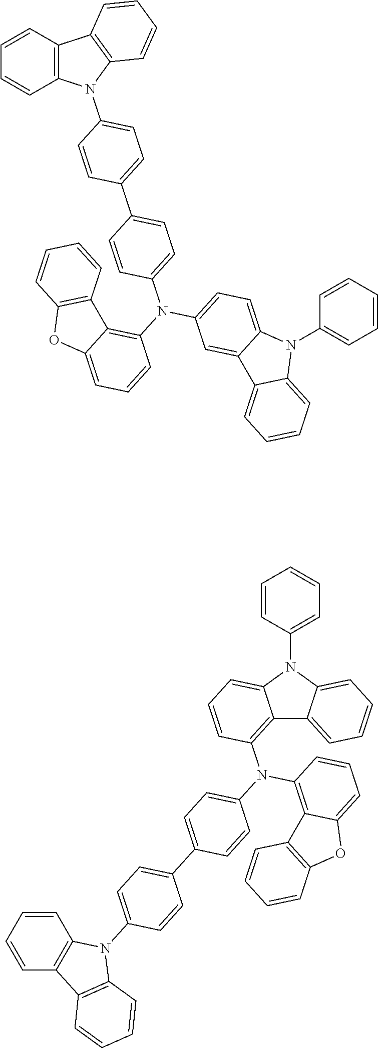 [SMILES: c1ccc(-n2c3ccccc3c3cc(N(c4ccc(-c5ccc(-n6c7ccccc7c7ccccc76)cc5)cc4)c4cccc5oc6ccccc6c45)ccc32)cc1 ; c1ccc(-n2c3ccccc3c3c(N(c4ccc(-c5ccc(-n6c7ccccc7c7ccccc76)cc5)cc4)c4cccc5oc6ccccc6c45)cccc32)cc1] 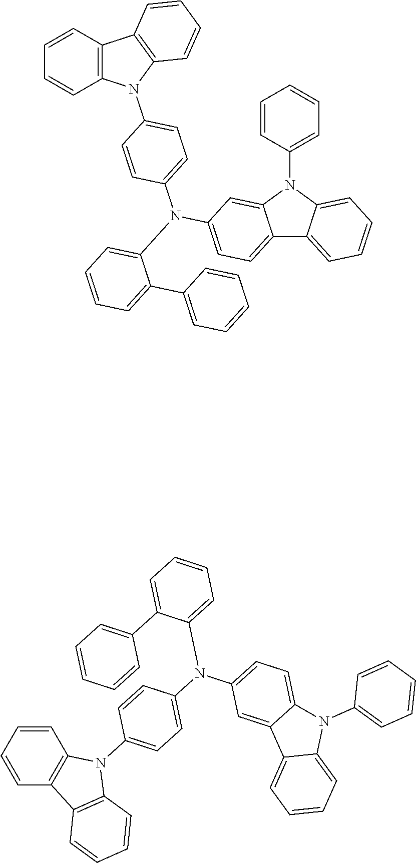 [SMILES: c1ccc(-c2ccccc2N(c2ccc(-n3c4ccccc4c4ccccc43)cc2)c2ccc3c4ccccc4n(-c4ccccc4)c3c2)cc1 ; c1ccc(-c2ccccc2N(c2ccc(-n3c4ccccc4c4ccccc43)cc2)c2ccc3c(c2)c2ccccc2n3-c2ccccc2)cc1] 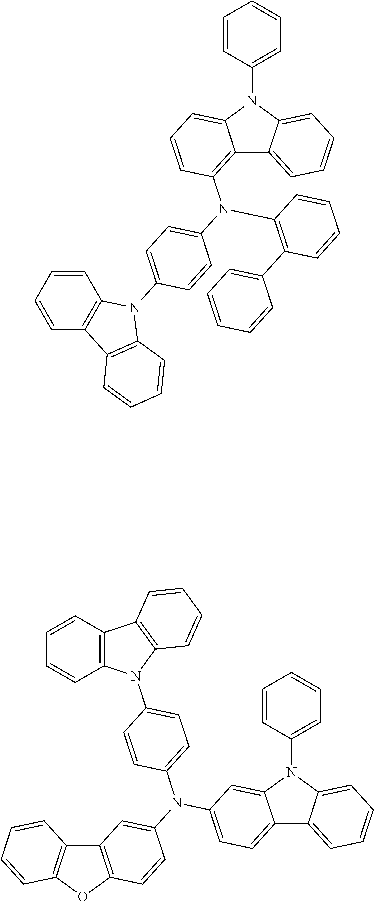 [SMILES: c1ccc(-c2ccccc2N(c2ccc(-n3c4ccccc4c4ccccc43)cc2)c2cccc3c2c2ccccc2n3-c2ccccc2)cc1 ; c1ccc(-n2c3ccccc3c3ccc(N(c4ccc(-n5c6ccccc6c6ccccc65)cc4)c4ccc5oc6ccccc6c5c4)cc32)cc1] 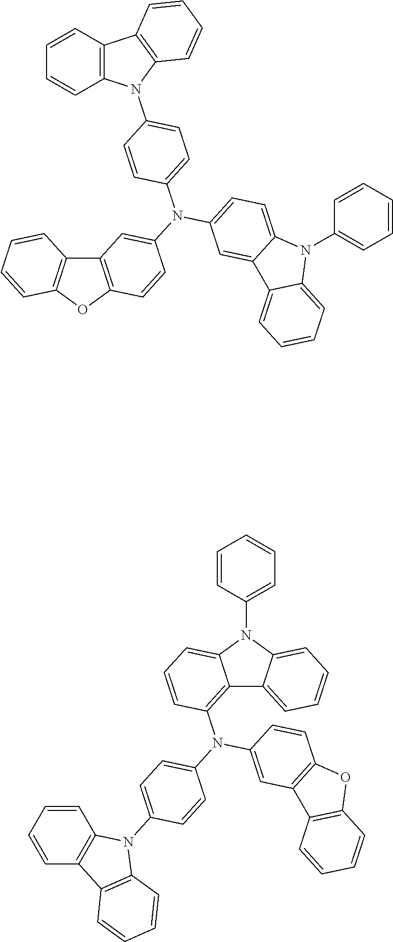 [SMILES: c1ccc(-n2c3ccccc3c3cc(N(c4ccc(-n5c6ccccc6c6ccccc65)cc4)c4ccc5oc6ccccc6c5c4)ccc32)cc1 ; c1ccc(-n2c3ccccc3c3c(N(c4ccc(-n5c6ccccc6c6ccccc65)cc4)c4ccc5oc6ccccc6c5c4)cccc32)cc1] 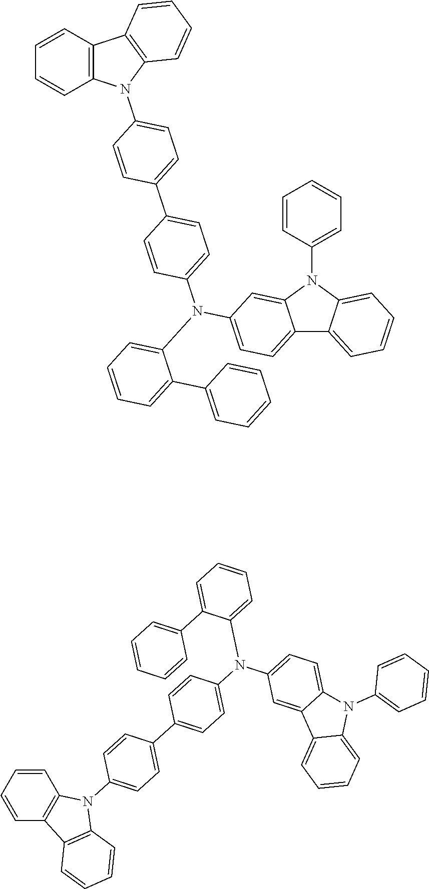 [SMILES: c1ccc(-c2ccccc2N(c2ccc(-c3ccc(-n4c5ccccc5c5ccccc54)cc3)cc2)c2ccc3c4ccccc4n(-c4ccccc4)c3c2)cc1 ; c1ccc(-c2ccccc2N(c2ccc(-c3ccc(-n4c5ccccc5c5ccccc54)cc3)cc2)c2ccc3c(c2)c2ccccc2n3-c2ccccc2)cc1] 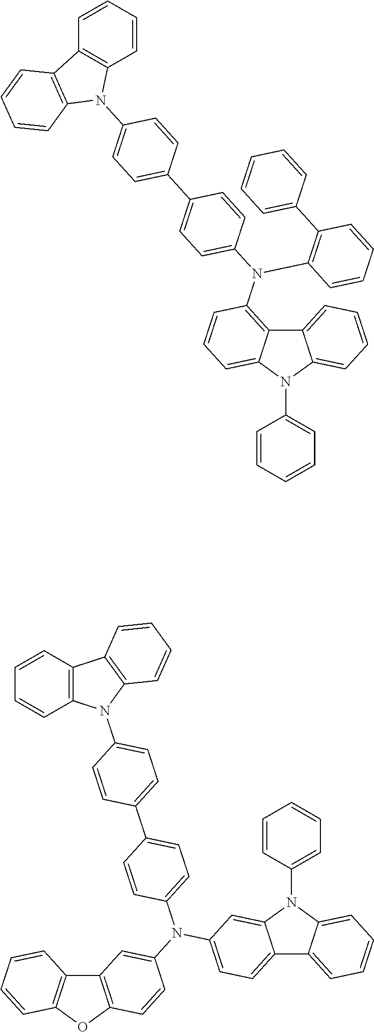 [SMILES: c1ccc(-c2ccccc2N(c2ccc(-c3ccc(-n4c5ccccc5c5ccccc54)cc3)cc2)c2cccc3c2c2ccccc2n3-c2ccccc2)cc1 ; c1ccc(-n2c3ccccc3c3ccc(N(c4ccc(-c5ccc(-n6c7ccccc7c7ccccc76)cc5)cc4)c4ccc5oc6ccccc6c5c4)cc32)cc1] 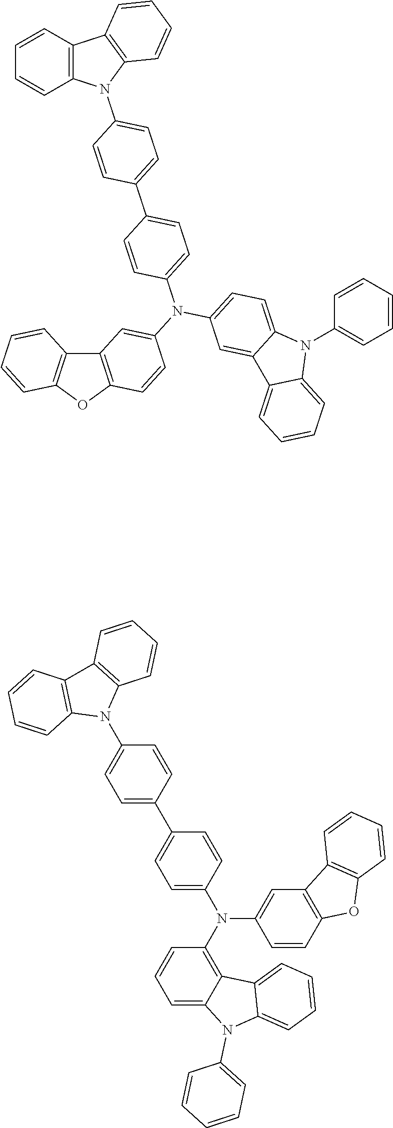 [SMILES: c1ccc(-n2c3ccccc3c3cc(N(c4ccc(-c5ccc(-n6c7ccccc7c7ccccc76)cc5)cc4)c4ccc5oc6ccccc6c5c4)ccc32)cc1 ; c1ccc(-n2c3ccccc3c3c(N(c4ccc(-c5ccc(-n6c7ccccc7c7ccccc76)cc5)cc4)c4ccc5oc6ccccc6c5c4)cccc32)cc1] 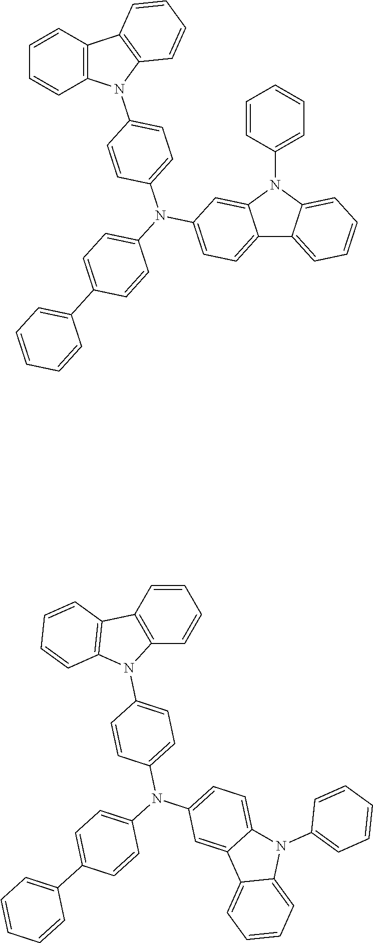 [SMILES: c1ccc(-c2ccc(N(c3ccc(-n4c5ccccc5c5ccccc54)cc3)c3ccc4c5ccccc5n(-c5ccccc5)c4c3)cc2)cc1 ; c1ccc(-c2ccc(N(c3ccc(-n4c5ccccc5c5ccccc54)cc3)c3ccc4c(c3)c3ccccc3n4-c3ccccc3)cc2)cc1] 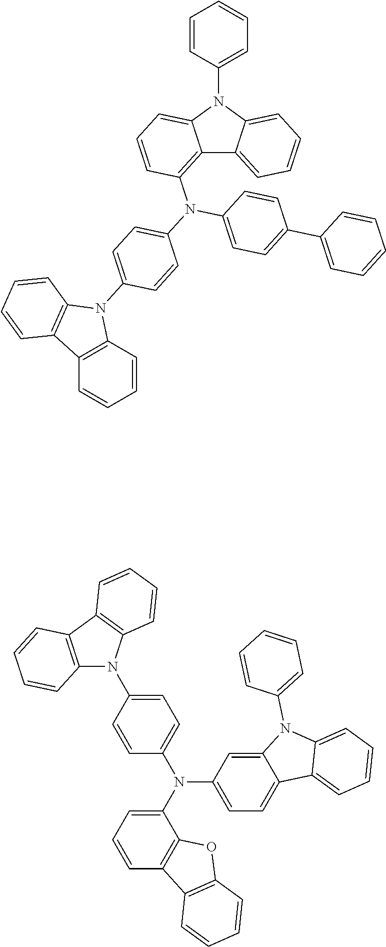 [SMILES: c1ccc(-c2ccc(N(c3ccc(-n4c5ccccc5c5ccccc54)cc3)c3cccc4c3c3ccccc3n4-c3ccccc3)cc2)cc1 ; c1ccc(-n2c3ccccc3c3ccc(N(c4ccc(-n5c6ccccc6c6ccccc65)cc4)c4cccc5c4oc4ccccc45)cc32)cc1] 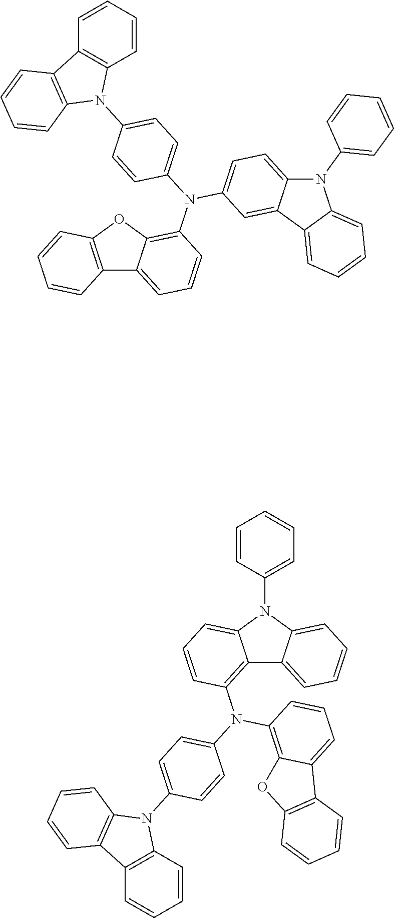 [SMILES: c1ccc(-n2c3ccccc3c3cc(N(c4ccc(-n5c6ccccc6c6ccccc65)cc4)c4cccc5c4oc4ccccc45)ccc32)cc1 ; c1ccc(-n2c3ccccc3c3c(N(c4ccc(-n5c6ccccc6c6ccccc65)cc4)c4cccc5c4oc4ccccc45)cccc32)cc1] 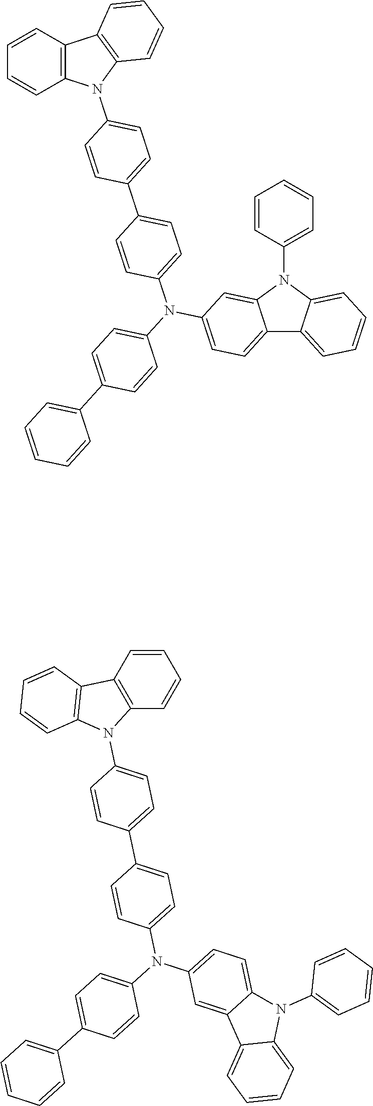 [SMILES: c1ccc(-c2ccc(N(c3ccc(-c4ccc(-n5c6ccccc6c6ccccc65)cc4)cc3)c3ccc4c5ccccc5n(-c5ccccc5)c4c3)cc2)cc1 ; c1ccc(-c2ccc(N(c3ccc(-c4ccc(-n5c6ccccc6c6ccccc65)cc4)cc3)c3ccc4c(c3)c3ccccc3n4-c3ccccc3)cc2)cc1] 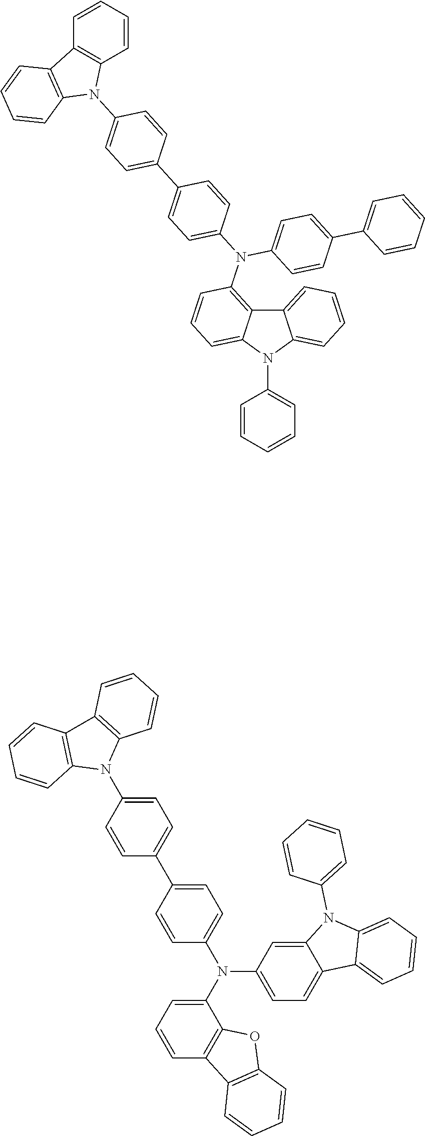 [SMILES: c1ccc(-c2ccc(N(c3ccc(-c4ccc(-n5c6ccccc6c6ccccc65)cc4)cc3)c3cccc4c3c3ccccc3n4-c3ccccc3)cc2)cc1 ; c1ccc(-n2c3ccccc3c3ccc(N(c4ccc(-c5ccc(-n6c7ccccc7c7ccccc76)cc5)cc4)c4cccc5c4oc4ccccc45)cc32)cc1] 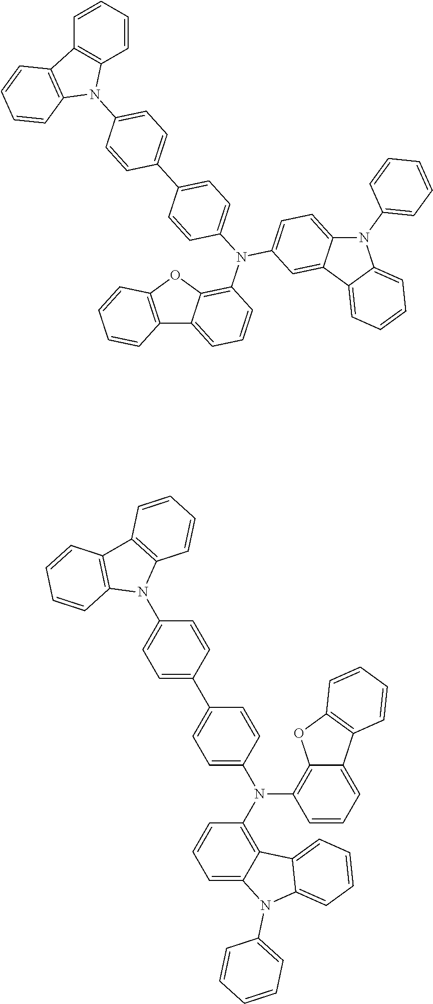 [SMILES: c1ccc(-n2c3ccccc3c3cc(N(c4ccc(-c5ccc(-n6c7ccccc7c7ccccc76)cc5)cc4)c4cccc5c4oc4ccccc45)ccc32)cc1 ; c1ccc(-n2c3ccccc3c3c(N(c4ccc(-c5ccc(-n6c7ccccc7c7ccccc76)cc5)cc4)c4cccc5c4oc4ccccc45)cccc32)cc1] 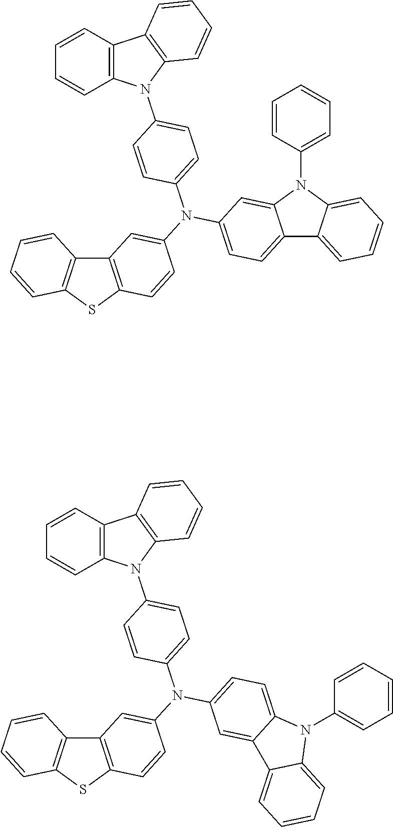 [SMILES: c1ccc(-n2c3ccccc3c3ccc(N(c4ccc(-n5c6ccccc6c6ccccc65)cc4)c4ccc5sc6ccccc6c5c4)cc32)cc1 ; c1ccc(-n2c3ccccc3c3cc(N(c4ccc(-n5c6ccccc6c6ccccc65)cc4)c4ccc5sc6ccccc6c5c4)ccc32)cc1] 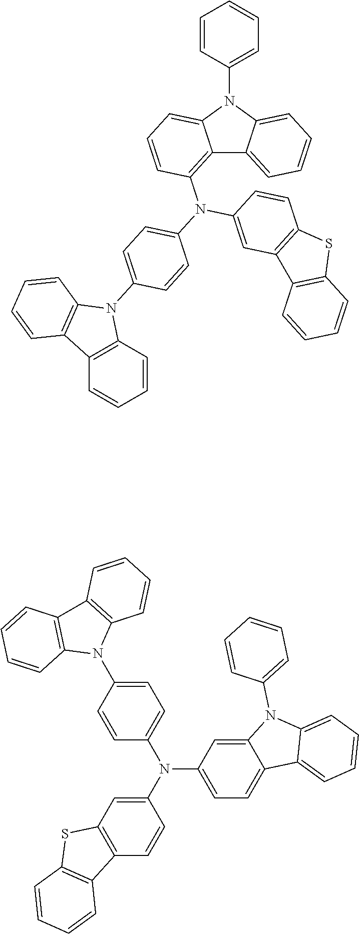 [SMILES: c1ccc(-n2c3ccccc3c3c(N(c4ccc(-n5c6ccccc6c6ccccc65)cc4)c4ccc5sc6ccccc6c5c4)cccc32)cc1 ; c1ccc(-n2c3ccccc3c3ccc(N(c4ccc(-n5c6ccccc6c6ccccc65)cc4)c4ccc5c(c4)sc4ccccc45)cc32)cc1] 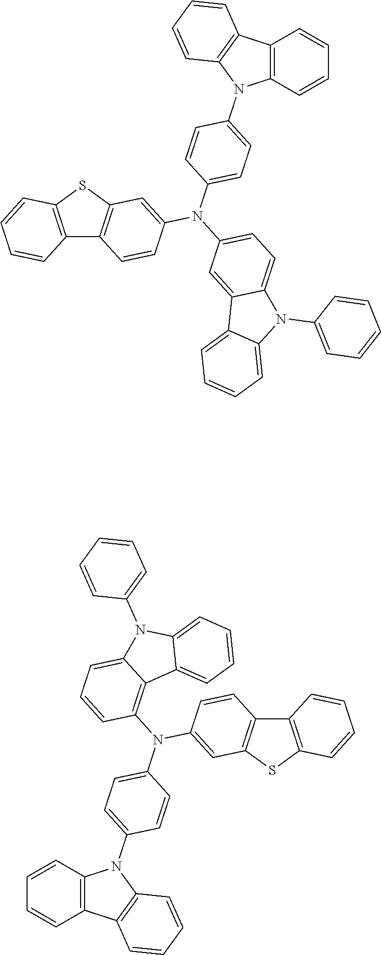 [SMILES: c1ccc(-n2c3ccccc3c3cc(N(c4ccc(-n5c6ccccc6c6ccccc65)cc4)c4ccc5c(c4)sc4ccccc45)ccc32)cc1 ; c1ccc(-n2c3ccccc3c3c(N(c4ccc(-n5c6ccccc6c6ccccc65)cc4)c4ccc5c(c4)sc4ccccc45)cccc32)cc1] 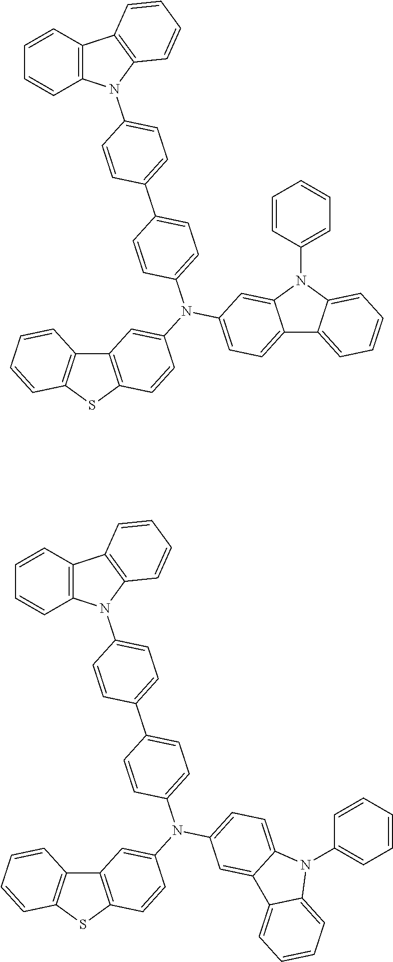 [SMILES: c1ccc(-n2c3ccccc3c3ccc(N(c4ccc(-c5ccc(-n6c7ccccc7c7ccccc76)cc5)cc4)c4ccc5sc6ccccc6c5c4)cc32)cc1 ; c1ccc(-n2c3ccccc3c3cc(N(c4ccc(-c5ccc(-n6c7ccccc7c7ccccc76)cc5)cc4)c4ccc5sc6ccccc6c5c4)ccc32)cc1] 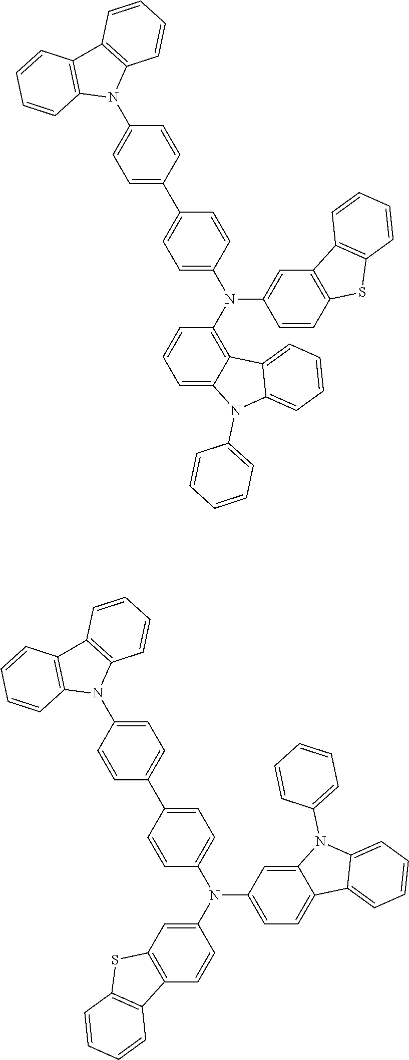 [SMILES: c1ccc(-n2c3ccccc3c3c(N(c4ccc(-c5ccc(-n6c7ccccc7c7ccccc76)cc5)cc4)c4ccc5sc6ccccc6c5c4)cccc32)cc1 ; c1ccc(-n2c3ccccc3c3ccc(N(c4ccc(-c5ccc(-n6c7ccccc7c7ccccc76)cc5)cc4)c4ccc5c(c4)sc4ccccc45)cc32)cc1] 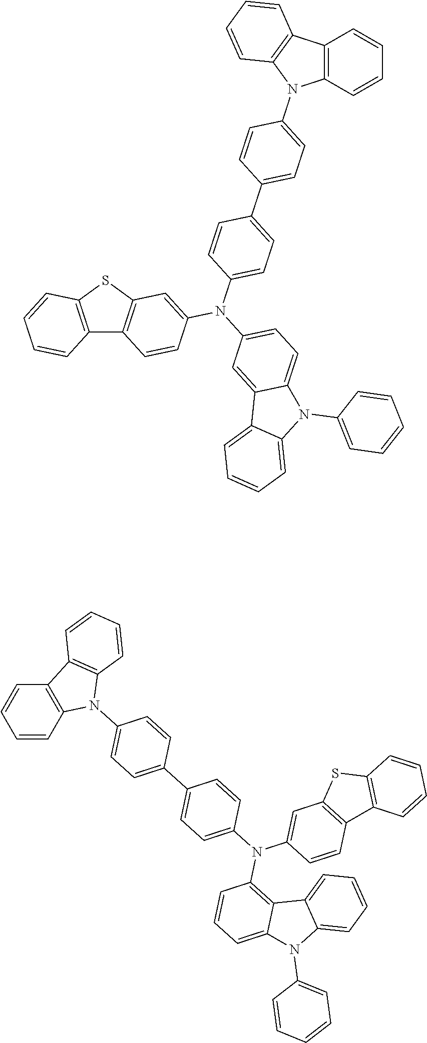 [SMILES: c1ccc(-n2c3ccccc3c3cc(N(c4ccc(-c5ccc(-n6c7ccccc7c7ccccc76)cc5)cc4)c4ccc5c(c4)sc4ccccc45)ccc32)cc1 ; c1ccc(-n2c3ccccc3c3c(N(c4ccc(-c5ccc(-n6c7ccccc7c7ccccc76)cc5)cc4)c4ccc5c(c4)sc4ccccc45)cccc32)cc1] 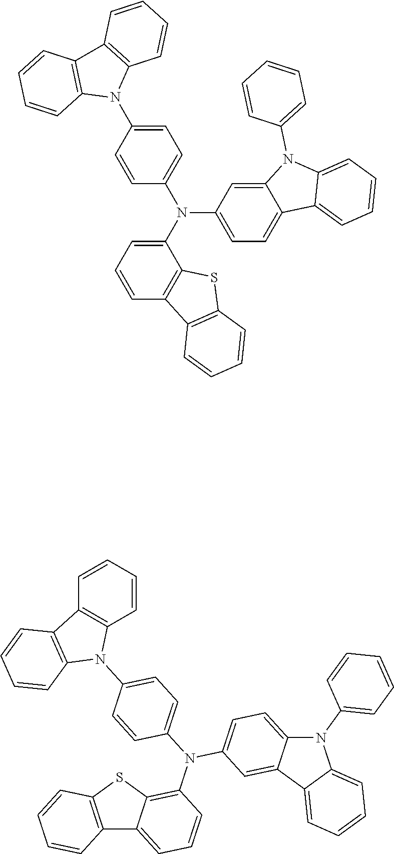 [SMILES: c1ccc(-n2c3ccccc3c3ccc(N(c4ccc(-n5c6ccccc6c6ccccc65)cc4)c4cccc5c4sc4ccccc45)cc32)cc1 ; c1ccc(-n2c3ccccc3c3cc(N(c4ccc(-n5c6ccccc6c6ccccc65)cc4)c4cccc5c4sc4ccccc45)ccc32)cc1] 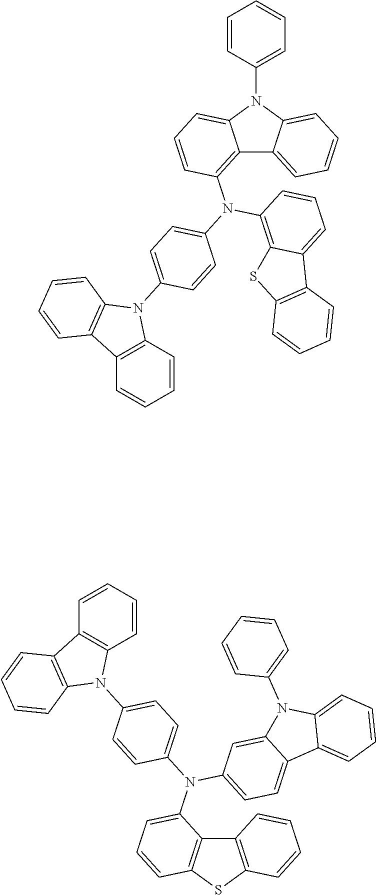 [SMILES: c1ccc(-n2c3ccccc3c3c(N(c4ccc(-n5c6ccccc6c6ccccc65)cc4)c4cccc5c4sc4ccccc45)cccc32)cc1 ; c1ccc(-n2c3ccccc3c3ccc(N(c4ccc(-n5c6ccccc6c6ccccc65)cc4)c4cccc5sc6ccccc6c45)cc32)cc1] 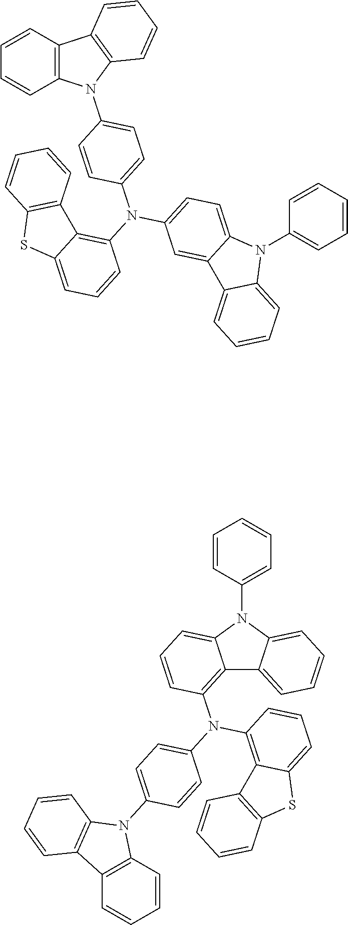 [SMILES: c1ccc(-n2c3ccccc3c3cc(N(c4ccc(-n5c6ccccc6c6ccccc65)cc4)c4cccc5sc6ccccc6c45)ccc32)cc1 ; c1ccc(-n2c3ccccc3c3c(N(c4ccc(-n5c6ccccc6c6ccccc65)cc4)c4cccc5sc6ccccc6c45)cccc32)cc1] 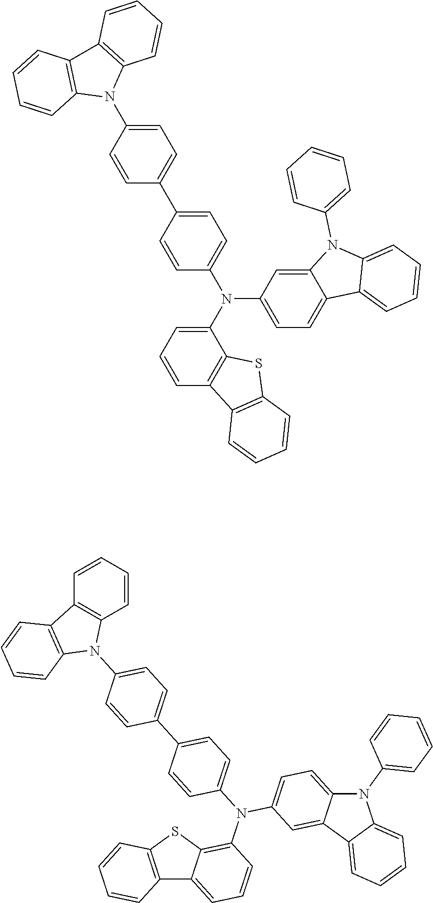 [SMILES: c1ccc(-n2c3ccccc3c3ccc(N(c4ccc(-c5ccc(-n6c7ccccc7c7ccccc76)cc5)cc4)c4cccc5c4sc4ccccc45)cc32)cc1 ; c1ccc(-n2c3ccccc3c3cc(N(c4ccc(-c5ccc(-n6c7ccccc7c7ccccc76)cc5)cc4)c4cccc5c4sc4ccccc45)ccc32)cc1] 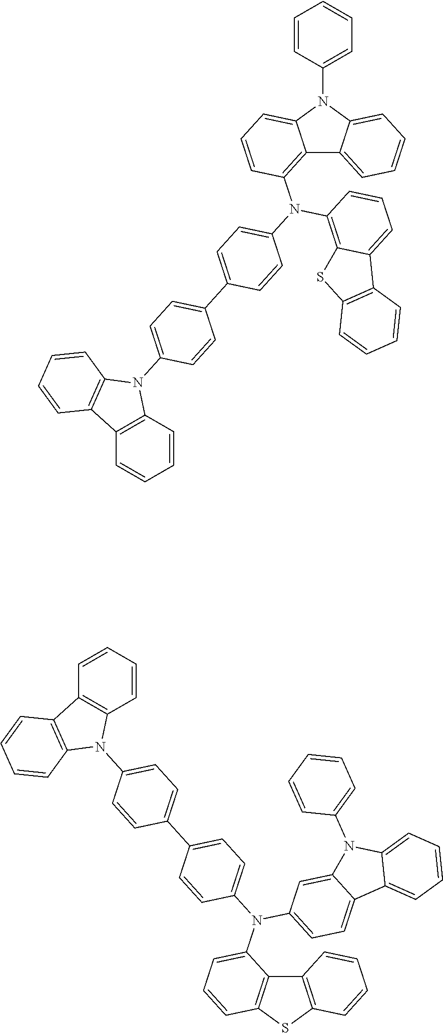 [SMILES: c1ccc(-n2c3ccccc3c3c(N(c4ccc(-c5ccc(-n6c7ccccc7c7ccccc76)cc5)cc4)c4cccc5c4sc4ccccc45)cccc32)cc1 ; c1ccc(-n2c3ccccc3c3ccc(N(c4ccc(-c5ccc(-n6c7ccccc7c7ccccc76)cc5)cc4)c4cccc5sc6ccccc6c45)cc32)cc1] 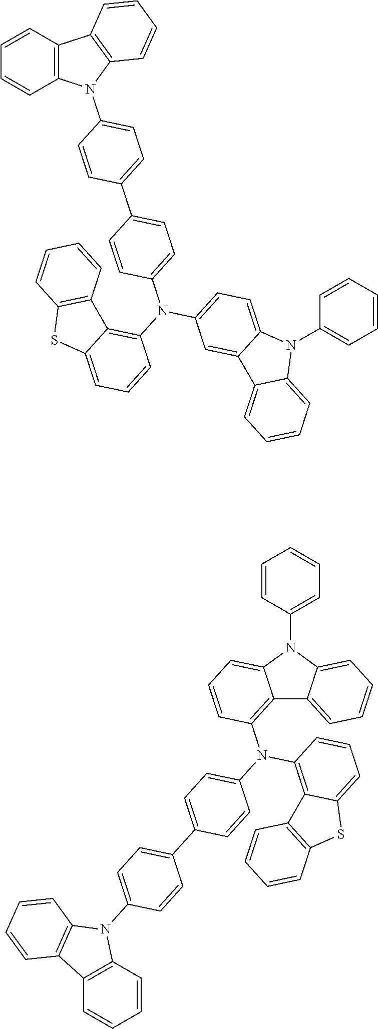 [SMILES: c1ccc(-n2c3ccccc3c3cc(N(c4ccc(-c5ccc(-n6c7ccccc7c7ccccc76)cc5)cc4)c4cccc5sc6ccccc6c45)ccc32)cc1 ; c1ccc(-n2c3ccccc3c3c(N(c4ccc(-c5ccc(-n6c7ccccc7c7ccccc76)cc5)cc4)c4cccc5sc6ccccc6c45)cccc32)cc1] 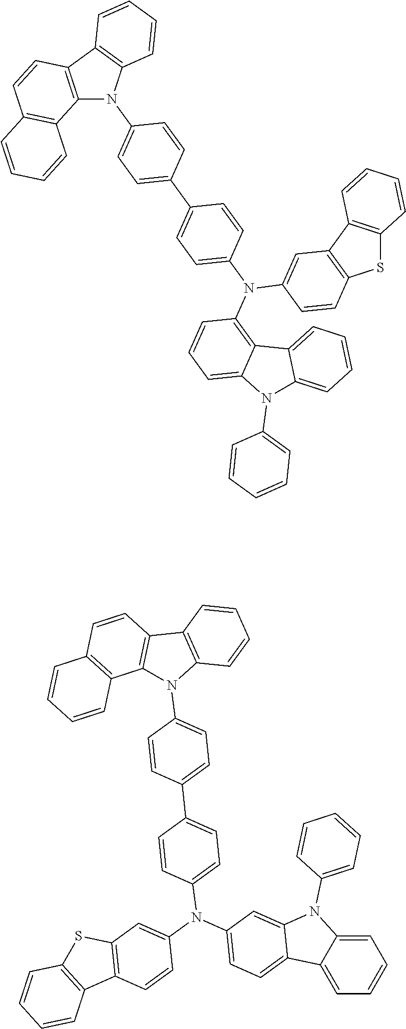 [SMILES: c1ccc(-n2c3ccccc3c3c(N(c4ccc(-c5ccc(-n6c7ccccc7c7ccc8ccccc8c76)cc5)cc4)c4ccc5sc6ccccc6c5c4)cccc32)cc1 ; c1ccc(-n2c3ccccc3c3ccc(N(c4ccc(-c5ccc(-n6c7ccccc7c7ccc8ccccc8c76)cc5)cc4)c4ccc5c(c4)sc4ccccc45)cc32)cc1] 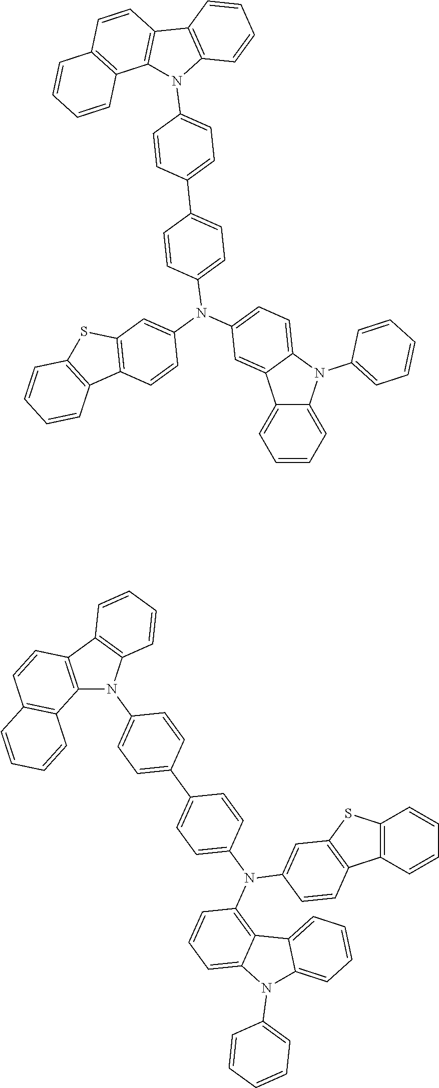 [SMILES: c1ccc(-n2c3ccccc3c3cc(N(c4ccc(-c5ccc(-n6c7ccccc7c7ccc8ccccc8c76)cc5)cc4)c4ccc5c(c4)sc4ccccc45)ccc32)cc1 ; c1ccc(-n2c3ccccc3c3c(N(c4ccc(-c5ccc(-n6c7ccccc7c7ccc8ccccc8c76)cc5)cc4)c4ccc5c(c4)sc4ccccc45)cccc32)cc1] 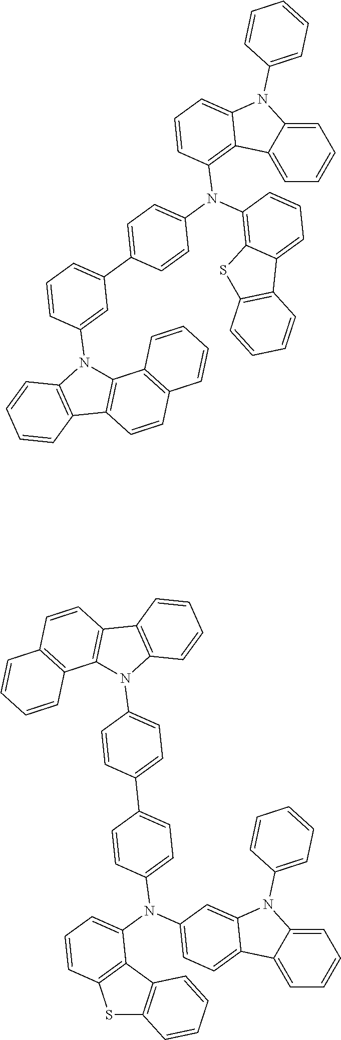 [SMILES: c1ccc(-n2c3ccccc3c3c(N(c4ccc(-c5cccc(-n6c7ccccc7c7ccc8ccccc8c76)c5)cc4)c4cccc5c4sc4ccccc45)cccc32)cc1 ; c1ccc(-n2c3ccccc3c3ccc(N(c4ccc(-c5ccc(-n6c7ccccc7c7ccc8ccccc8c76)cc5)cc4)c4cccc5sc6ccccc6c45)cc32)cc1] 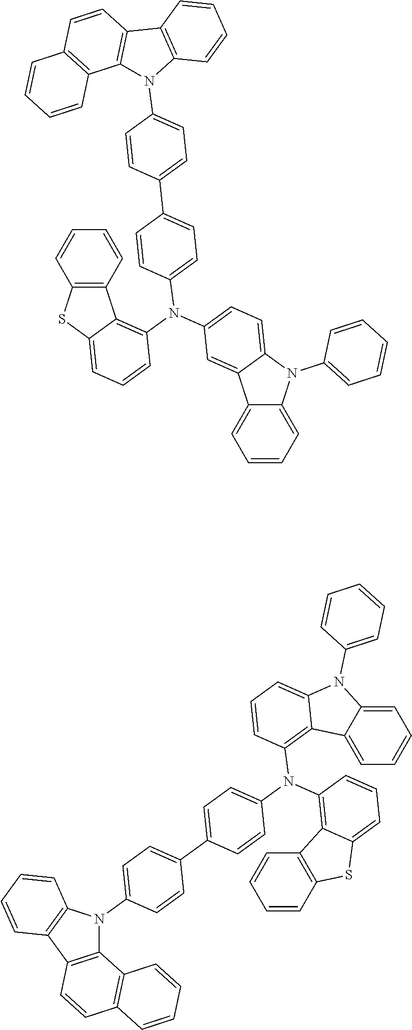 [SMILES: c1ccc(-n2c3ccccc3c3cc(N(c4ccc(-c5ccc(-n6c7ccccc7c7ccc8ccccc8c76)cc5)cc4)c4cccc5sc6ccccc6c45)ccc32)cc1 ; c1ccc(-n2c3ccccc3c3c(N(c4ccc(-c5ccc(-n6c7ccccc7c7ccc8ccccc8c76)cc5)cc4)c4cccc5sc6ccccc6c45)cccc32)cc1] 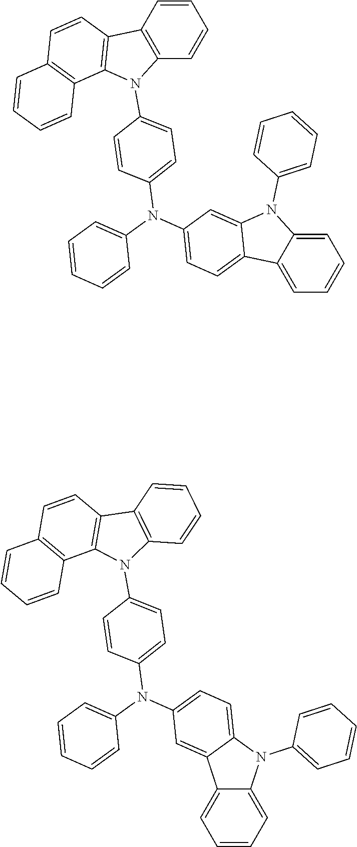 [SMILES: c1ccc(N(c2ccc(-n3c4ccccc4c4ccc5ccccc5c43)cc2)c2ccc3c4ccccc4n(-c4ccccc4)c3c2)cc1 ; c1ccc(N(c2ccc(-n3c4ccccc4c4ccc5ccccc5c43)cc2)c2ccc3c(c2)c2ccccc2n3-c2ccccc2)cc1] 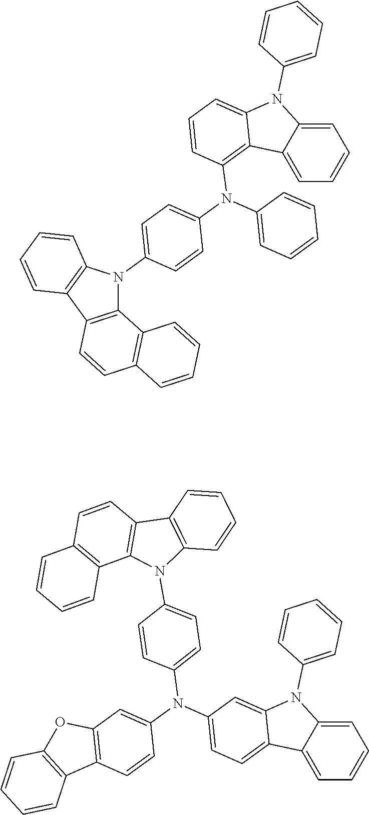 [SMILES: c1ccc(N(c2ccc(-n3c4ccccc4c4ccc5ccccc5c43)cc2)c2cccc3c2c2ccccc2n3-c2ccccc2)cc1 ; c1ccc(-n2c3ccccc3c3ccc(N(c4ccc(-n5c6ccccc6c6ccc7ccccc7c65)cc4)c4ccc5c(c4)oc4ccccc45)cc32)cc1] 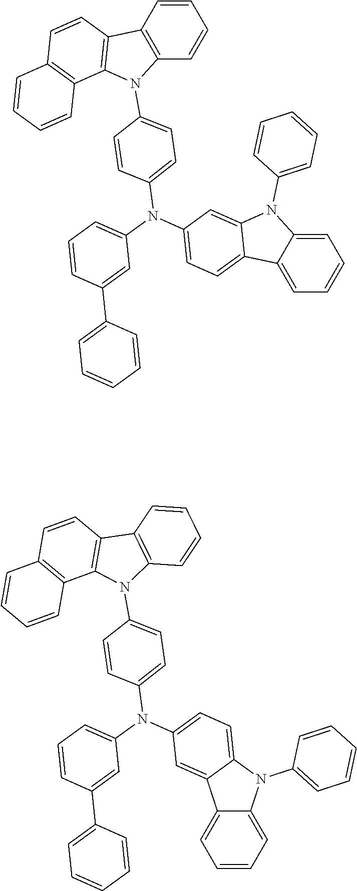 [SMILES: c1ccc(-c2cccc(N(c3ccc(-n4c5ccccc5c5ccc6ccccc6c54)cc3)c3ccc4c5ccccc5n(-c5ccccc5)c4c3)c2)cc1 ; c1ccc(-c2cccc(N(c3ccc(-n4c5ccccc5c5ccc6ccccc6c54)cc3)c3ccc4c(c3)c3ccccc3n4-c3ccccc3)c2)cc1] 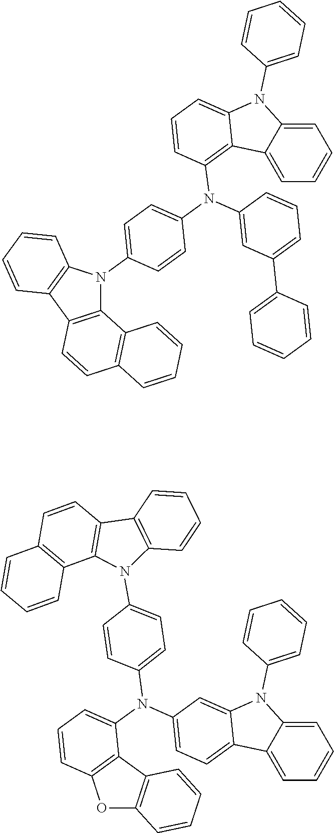 [SMILES: c1ccc(-c2cccc(N(c3ccc(-n4c5ccccc5c5ccc6ccccc6c54)cc3)c3cccc4c3c3ccccc3n4-c3ccccc3)c2)cc1 ; c1ccc(-n2c3ccccc3c3ccc(N(c4ccc(-n5c6ccccc6c6ccc7ccccc7c65)cc4)c4cccc5oc6ccccc6c45)cc32)cc1] 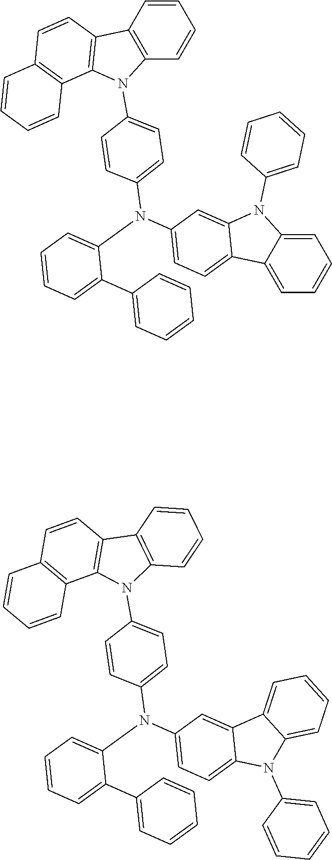 [SMILES: c1ccc(-c2ccccc2N(c2ccc(-n3c4ccccc4c4ccc5ccccc5c43)cc2)c2ccc3c4ccccc4n(-c4ccccc4)c3c2)cc1 ; c1ccc(-c2ccccc2N(c2ccc(-n3c4ccccc4c4ccc5ccccc5c43)cc2)c2ccc3c(c2)c2ccccc2n3-c2ccccc2)cc1] 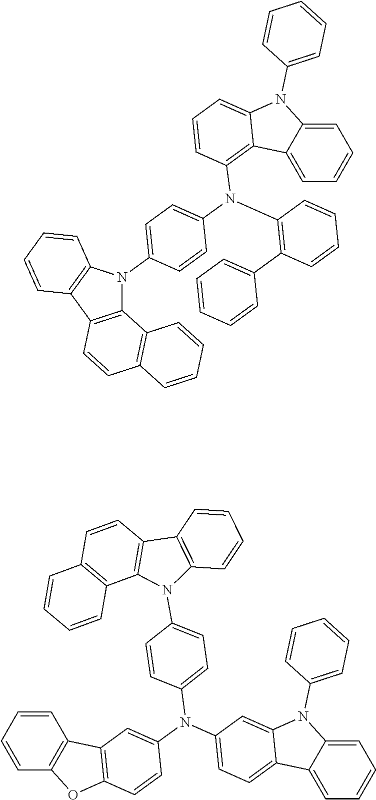 [SMILES: c1ccc(-c2ccccc2N(c2ccc(-n3c4ccccc4c4ccc5ccccc5c43)cc2)c2cccc3c2c2ccccc2n3-c2ccccc2)cc1 ; c1ccc(-n2c3ccccc3c3ccc(N(c4ccc(-n5c6ccccc6c6ccc7ccccc7c65)cc4)c4ccc5oc6ccccc6c5c4)cc32)cc1] 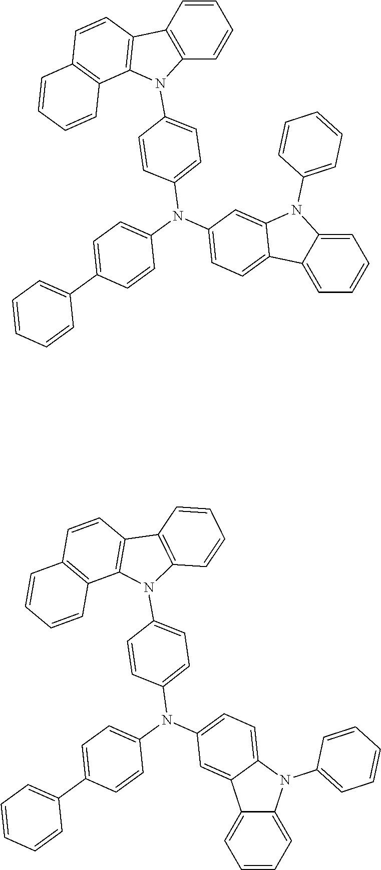 [SMILES: c1ccc(-c2ccc(N(c3ccc(-n4c5ccccc5c5ccc6ccccc6c54)cc3)c3ccc4c5ccccc5n(-c5ccccc5)c4c3)cc2)cc1 ; c1ccc(-c2ccc(N(c3ccc(-n4c5ccccc5c5ccc6ccccc6c54)cc3)c3ccc4c(c3)c3ccccc3n4-c3ccccc3)cc2)cc1] 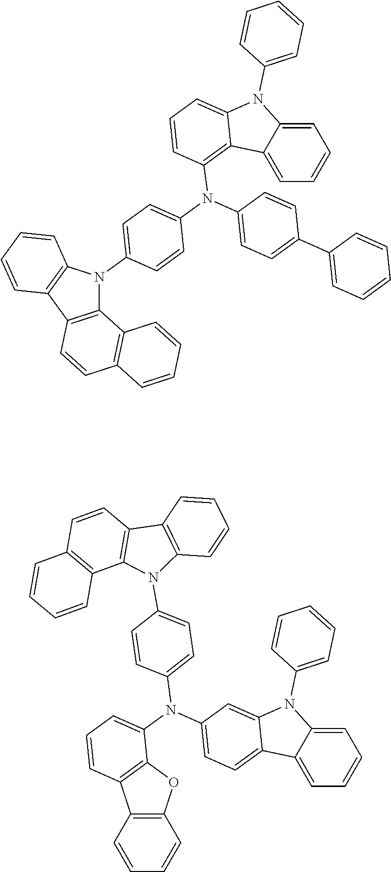 [SMILES: c1ccc(-c2ccc(N(c3ccc(-n4c5ccccc5c5ccc6ccccc6c54)cc3)c3cccc4c3c3ccccc3n4-c3ccccc3)cc2)cc1 ; c1ccc(-n2c3ccccc3c3ccc(N(c4ccc(-n5c6ccccc6c6ccc7ccccc7c65)cc4)c4cccc5c4oc4ccccc45)cc32)cc1] 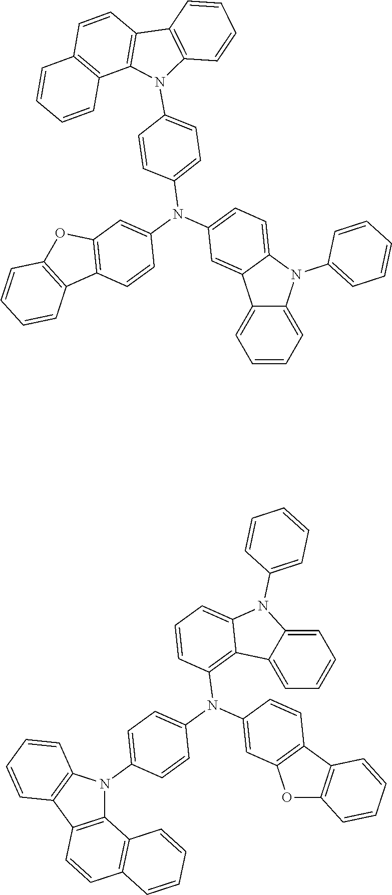 [SMILES: c1ccc(-n2c3ccccc3c3cc(N(c4ccc(-n5c6ccccc6c6ccc7ccccc7c65)cc4)c4ccc5c(c4)oc4ccccc45)ccc32)cc1 ; c1ccc(-n2c3ccccc3c3c(N(c4ccc(-n5c6ccccc6c6ccc7ccccc7c65)cc4)c4ccc5c(c4)oc4ccccc45)cccc32)cc1] 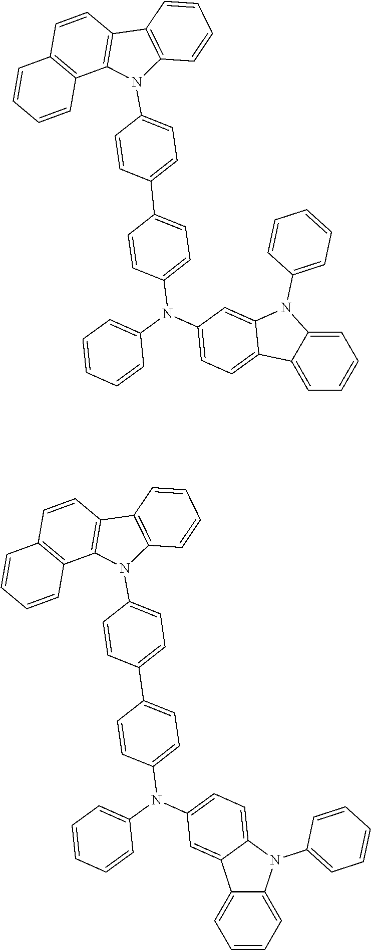 [SMILES: c1ccc(N(c2ccc(-c3ccc(-n4c5ccccc5c5ccc6ccccc6c54)cc3)cc2)c2ccc3c4ccccc4n(-c4ccccc4)c3c2)cc1 ; c1ccc(N(c2ccc(-c3ccc(-n4c5ccccc5c5ccc6ccccc6c54)cc3)cc2)c2ccc3c(c2)c2ccccc2n3-c2ccccc2)cc1] 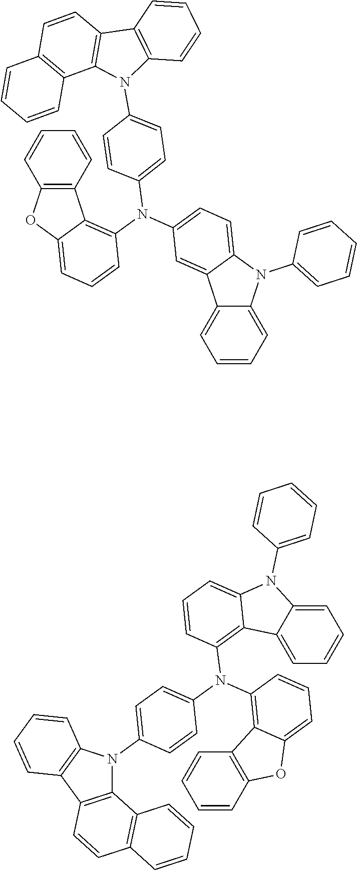 [SMILES: c1ccc(-n2c3ccccc3c3cc(N(c4ccc(-n5c6ccccc6c6ccc7ccccc7c65)cc4)c4cccc5oc6ccccc6c45)ccc32)cc1 ; c1ccc(-n2c3ccccc3c3c(N(c4ccc(-n5c6ccccc6c6ccc7ccccc7c65)cc4)c4cccc5oc6ccccc6c45)cccc32)cc1] 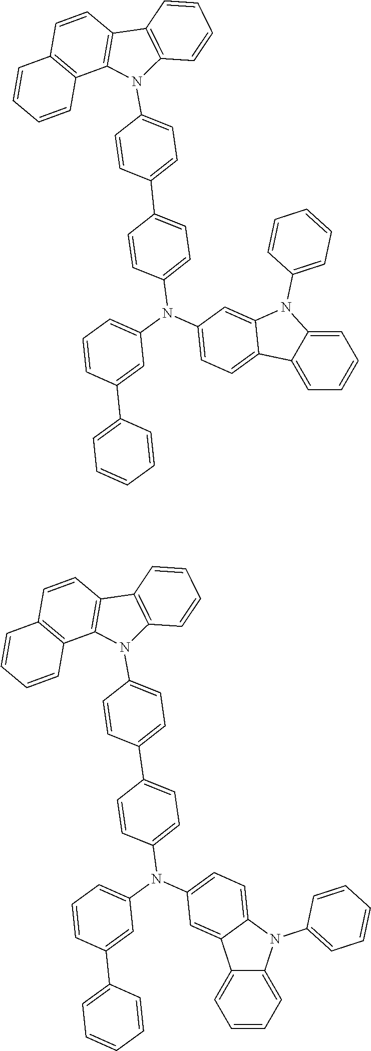 [SMILES: c1ccc(-c2cccc(N(c3ccc(-c4ccc(-n5c6ccccc6c6ccc7ccccc7c65)cc4)cc3)c3ccc4c5ccccc5n(-c5ccccc5)c4c3)c2)cc1 ; c1ccc(-c2cccc(N(c3ccc(-c4ccc(-n5c6ccccc6c6ccc7ccccc7c65)cc4)cc3)c3ccc4c(c3)c3ccccc3n4-c3ccccc3)c2)cc1] 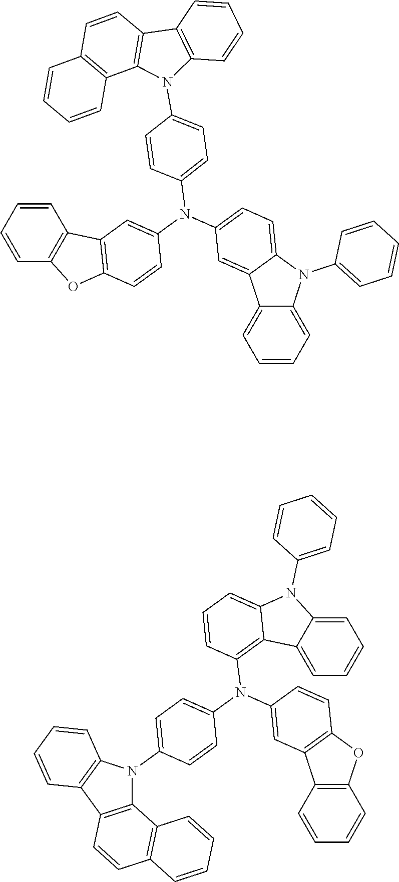 [SMILES: c1ccc(-n2c3ccccc3c3cc(N(c4ccc(-n5c6ccccc6c6ccc7ccccc7c65)cc4)c4ccc5oc6ccccc6c5c4)ccc32)cc1 ; c1ccc(-n2c3ccccc3c3c(N(c4ccc(-n5c6ccccc6c6ccc7ccccc7c65)cc4)c4ccc5oc6ccccc6c5c4)cccc32)cc1] 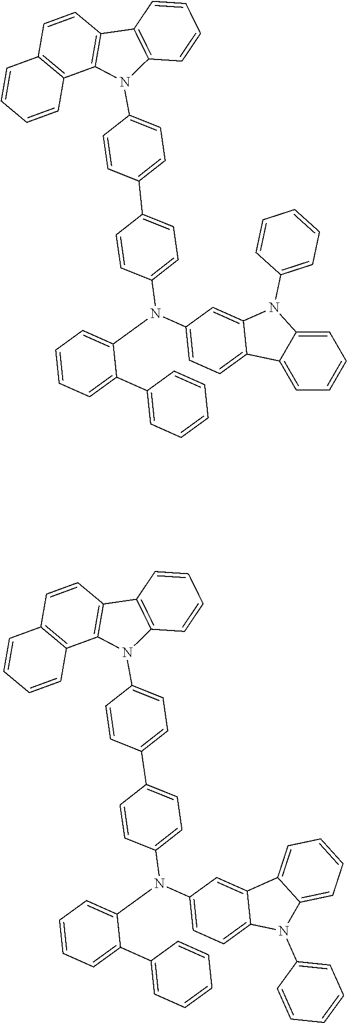 [SMILES: c1ccc(-c2ccccc2N(c2ccc(-c3ccc(-n4c5ccccc5c5ccc6ccccc6c54)cc3)cc2)c2ccc3c4ccccc4n(-c4ccccc4)c3c2)cc1 ; c1ccc(-c2ccccc2N(c2ccc(-c3ccc(-n4c5ccccc5c5ccc6ccccc6c54)cc3)cc2)c2ccc3c(c2)c2ccccc2n3-c2ccccc2)cc1] 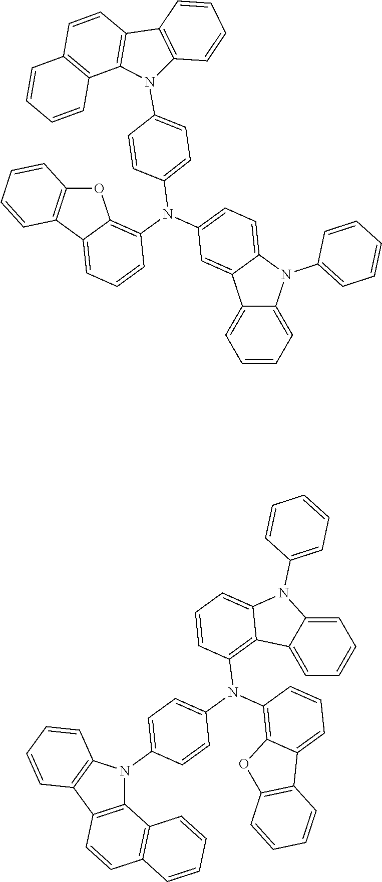 [SMILES: c1ccc(-n2c3ccccc3c3cc(N(c4ccc(-n5c6ccccc6c6ccc7ccccc7c65)cc4)c4cccc5c4oc4ccccc45)ccc32)cc1 ; c1ccc(-n2c3ccccc3c3c(N(c4ccc(-n5c6ccccc6c6ccc7ccccc7c65)cc4)c4cccc5c4oc4ccccc45)cccc32)cc1] 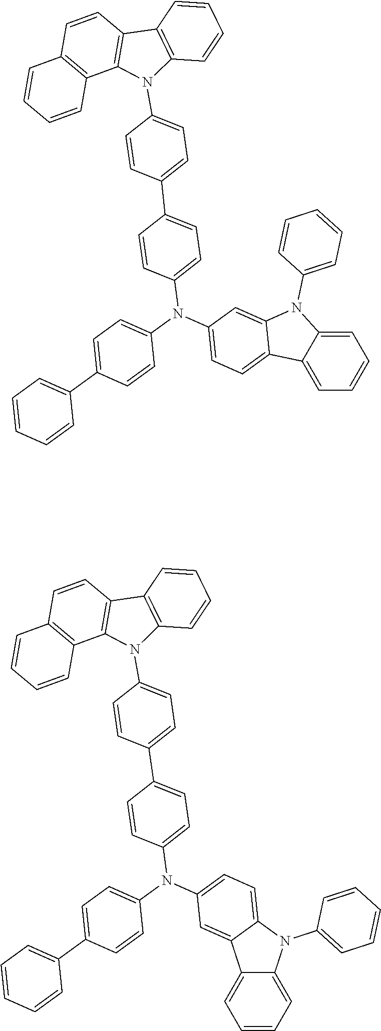 [SMILES: c1ccc(-c2ccc(N(c3ccc(-c4ccc(-n5c6ccccc6c6ccc7ccccc7c65)cc4)cc3)c3ccc4c5ccccc5n(-c5ccccc5)c4c3)cc2)cc1 ; c1ccc(-c2ccc(N(c3ccc(-c4ccc(-n5c6ccccc6c6ccc7ccccc7c65)cc4)cc3)c3ccc4c(c3)c3ccccc3n4-c3ccccc3)cc2)cc1] 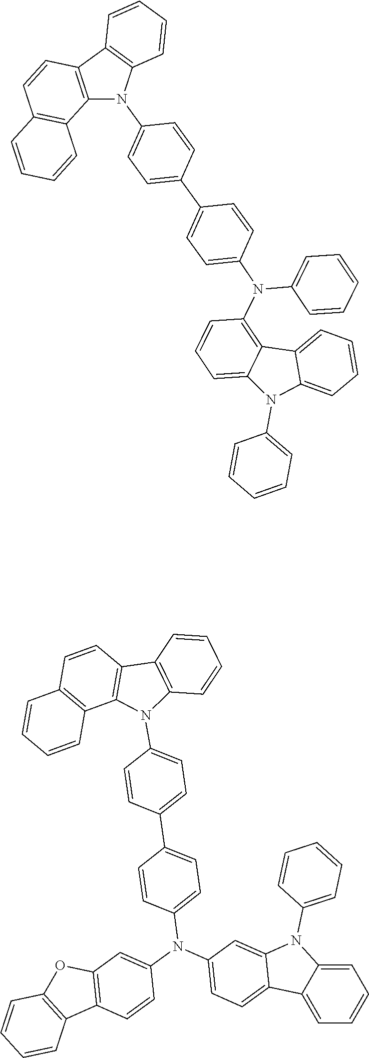 [SMILES: c1ccc(N(c2ccc(-c3ccc(-n4c5ccccc5c5ccc6ccccc6c54)cc3)cc2)c2cccc3c2c2ccccc2n3-c2ccccc2)cc1 ; c1ccc(-n2c3ccccc3c3ccc(N(c4ccc(-c5ccc(-n6c7ccccc7c7ccc8ccccc8c76)cc5)cc4)c4ccc5c(c4)oc4ccccc45)cc32)cc1] 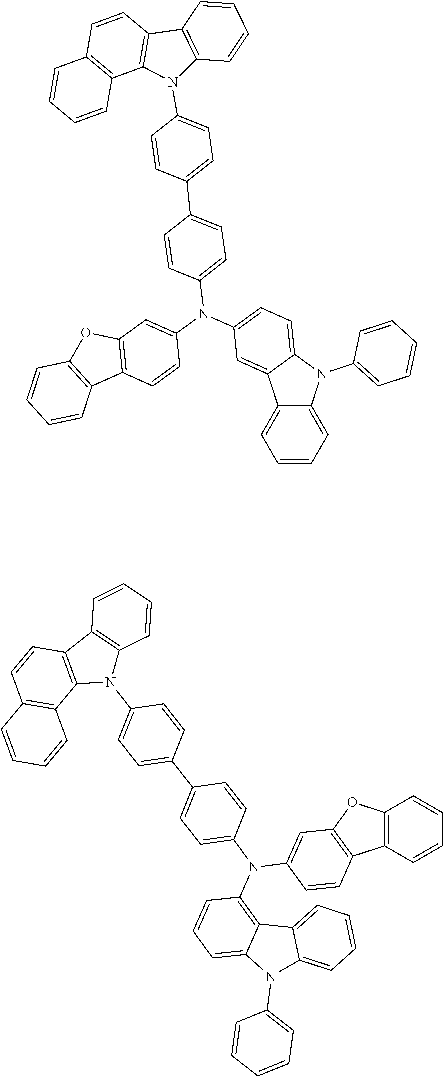 [SMILES: c1ccc(-n2c3ccccc3c3cc(N(c4ccc(-c5ccc(-n6c7ccccc7c7ccc8ccccc8c76)cc5)cc4)c4ccc5c(c4)oc4ccccc45)ccc32)cc1 ; c1ccc(-n2c3ccccc3c3c(N(c4ccc(-c5ccc(-n6c7ccccc7c7ccc8ccccc8c76)cc5)cc4)c4ccc5c(c4)oc4ccccc45)cccc32)cc1] 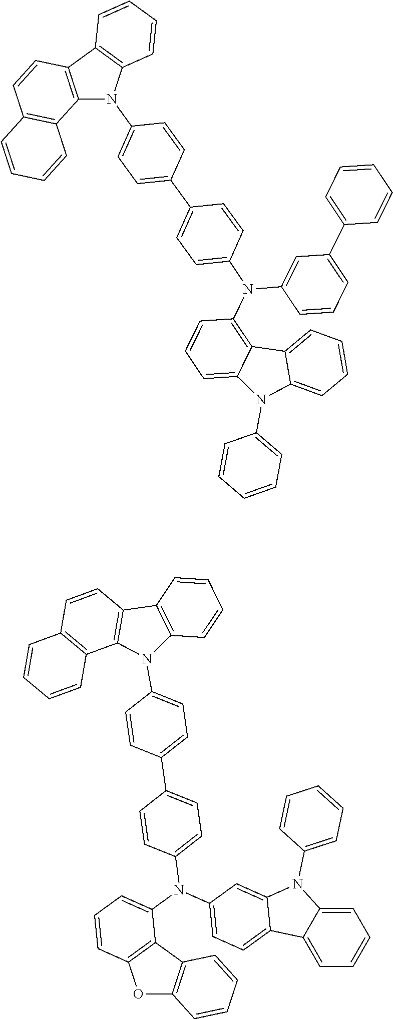 [SMILES: c1ccc(-c2cccc(N(c3ccc(-c4ccc(-n5c6ccccc6c6ccc7ccccc7c65)cc4)cc3)c3cccc4c3c3ccccc3n4-c3ccccc3)c2)cc1 ; c1ccc(-n2c3ccccc3c3ccc(N(c4ccc(-c5ccc(-n6c7ccccc7c7ccc8ccccc8c76)cc5)cc4)c4cccc5oc6ccccc6c45)cc32)cc1] 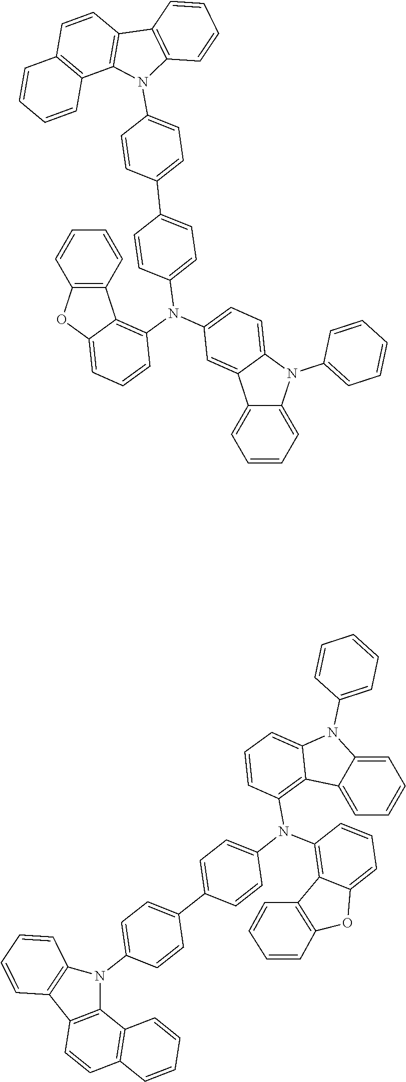 [SMILES: c1ccc(-n2c3ccccc3c3cc(N(c4ccc(-c5ccc(-n6c7ccccc7c7ccc8ccccc8c76)cc5)cc4)c4cccc5oc6ccccc6c45)ccc32)cc1 ; c1ccc(-n2c3ccccc3c3c(N(c4ccc(-c5ccc(-n6c7ccccc7c7ccc8ccccc8c76)cc5)cc4)c4cccc5oc6ccccc6c45)cccc32)cc1] 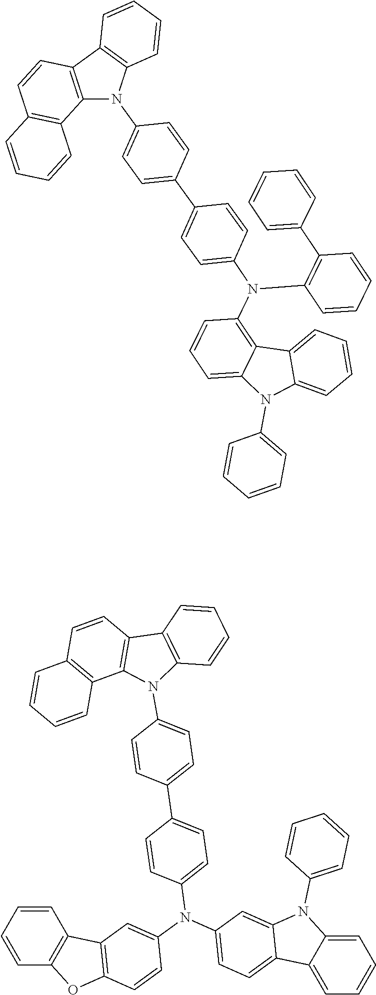 [SMILES: c1ccc(-c2ccccc2N(c2ccc(-c3ccc(-n4c5ccccc5c5ccc6ccccc6c54)cc3)cc2)c2cccc3c2c2ccccc2n3-c2ccccc2)cc1 ; c1ccc(-n2c3ccccc3c3ccc(N(c4ccc(-c5ccc(-n6c7ccccc7c7ccc8ccccc8c76)cc5)cc4)c4ccc5oc6ccccc6c5c4)cc32)cc1] 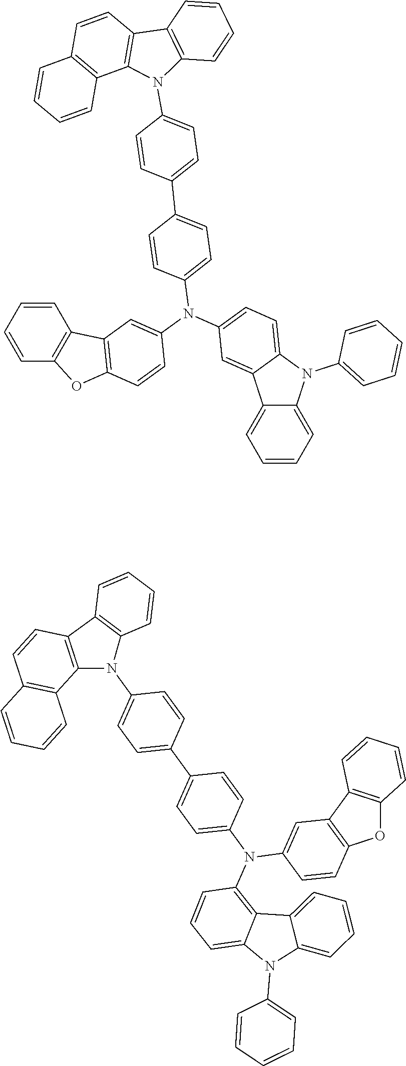 [SMILES: c1ccc(-n2c3ccccc3c3cc(N(c4ccc(-c5ccc(-n6c7ccccc7c7ccc8ccccc8c76)cc5)cc4)c4ccc5oc6ccccc6c5c4)ccc32)cc1 ; c1ccc(-n2c3ccccc3c3c(N(c4ccc(-c5ccc(-n6c7ccccc7c7ccc8ccccc8c76)cc5)cc4)c4ccc5oc6ccccc6c5c4)cccc32)cc1] 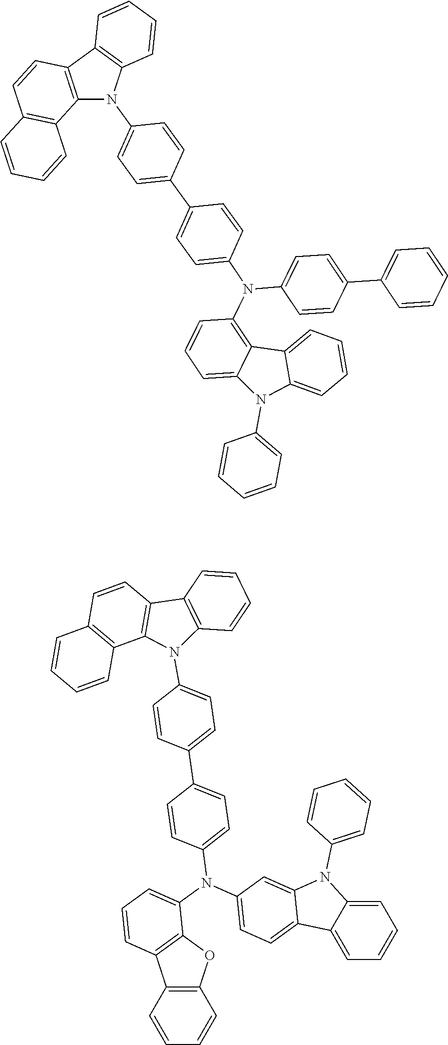 [SMILES: c1ccc(-c2ccc(N(c3ccc(-c4ccc(-n5c6ccccc6c6ccc7ccccc7c65)cc4)cc3)c3cccc4c3c3ccccc3n4-c3ccccc3)cc2)cc1 ; c1ccc(-n2c3ccccc3c3ccc(N(c4ccc(-c5ccc(-n6c7ccccc7c7ccc8ccccc8c76)cc5)cc4)c4cccc5c4oc4ccccc45)cc32)cc1] 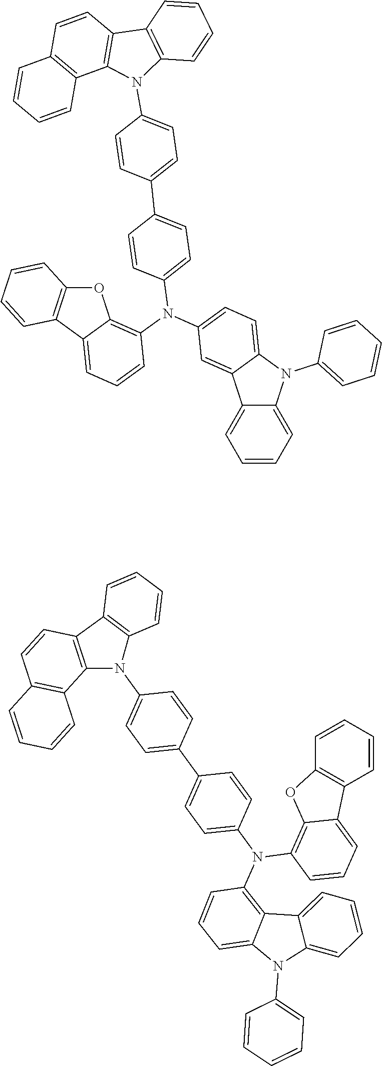 [SMILES: c1ccc(-n2c3ccccc3c3cc(N(c4ccc(-c5ccc(-n6c7ccccc7c7ccc8ccccc8c76)cc5)cc4)c4cccc5c4oc4ccccc45)ccc32)cc1 ; c1ccc(-n2c3ccccc3c3c(N(c4ccc(-c5ccc(-n6c7ccccc7c7ccc8ccccc8c76)cc5)cc4)c4cccc5c4oc4ccccc45)cccc32)cc1] 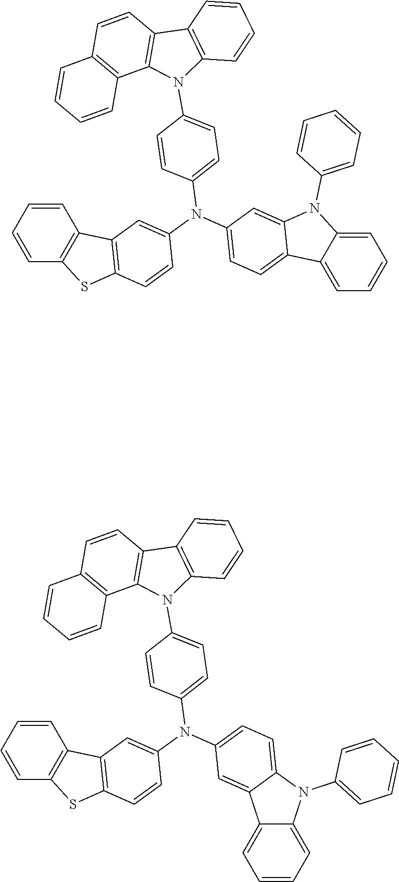 [SMILES: c1ccc(-n2c3ccccc3c3ccc(N(c4ccc(-n5c6ccccc6c6ccc7ccccc7c65)cc4)c4ccc5sc6ccccc6c5c4)cc32)cc1 ; c1ccc(-n2c3ccccc3c3cc(N(c4ccc(-n5c6ccccc6c6ccc7ccccc7c65)cc4)c4ccc5sc6ccccc6c5c4)ccc32)cc1] 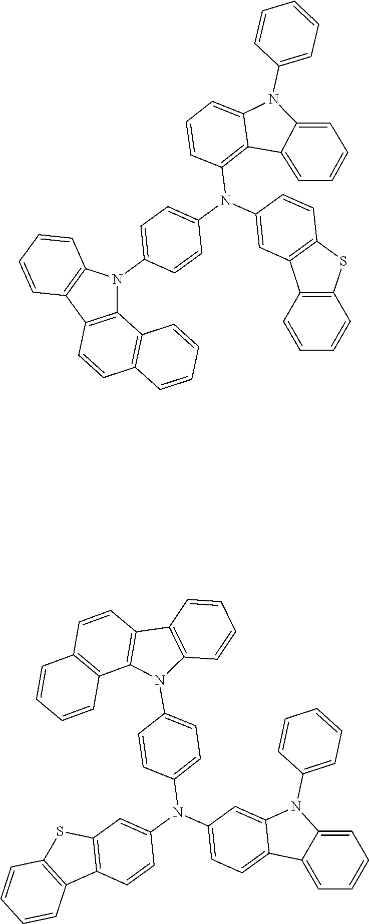 [SMILES: c1ccc(-n2c3ccccc3c3c(N(c4ccc(-n5c6ccccc6c6ccc7ccccc7c65)cc4)c4ccc5sc6ccccc6c5c4)cccc32)cc1 ; c1ccc(-n2c3ccccc3c3ccc(N(c4ccc(-n5c6ccccc6c6ccc7ccccc7c65)cc4)c4ccc5c(c4)sc4ccccc45)cc32)cc1] 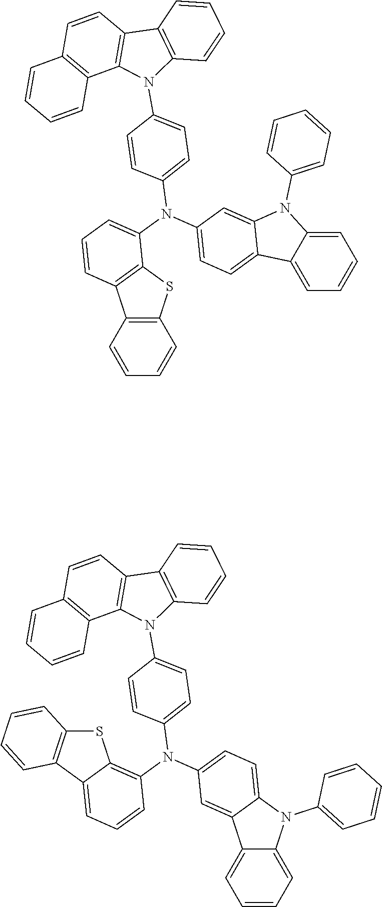 [SMILES: c1ccc(-n2c3ccccc3c3ccc(N(c4ccc(-n5c6ccccc6c6ccc7ccccc7c65)cc4)c4cccc5c4sc4ccccc45)cc32)cc1 ; c1ccc(-n2c3ccccc3c3cc(N(c4ccc(-n5c6ccccc6c6ccc7ccccc7c65)cc4)c4cccc5c4sc4ccccc45)ccc32)cc1] 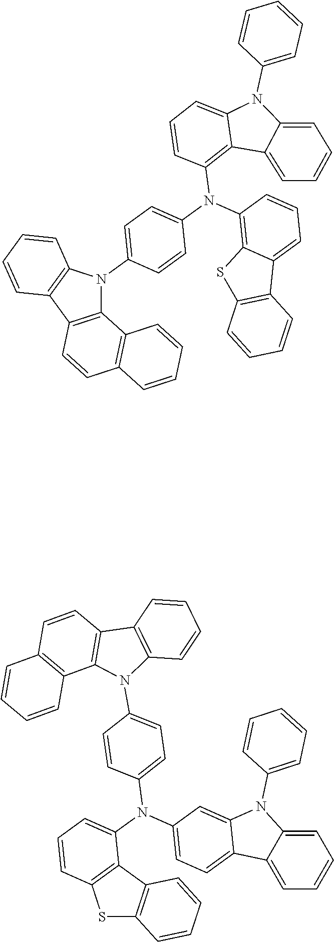 [SMILES: c1ccc(-n2c3ccccc3c3c(N(c4ccc(-n5c6ccccc6c6ccc7ccccc7c65)cc4)c4cccc5c4sc4ccccc45)cccc32)cc1 ; c1ccc(-n2c3ccccc3c3ccc(N(c4ccc(-n5c6ccccc6c6ccc7ccccc7c65)cc4)c4cccc5sc6ccccc6c45)cc32)cc1] 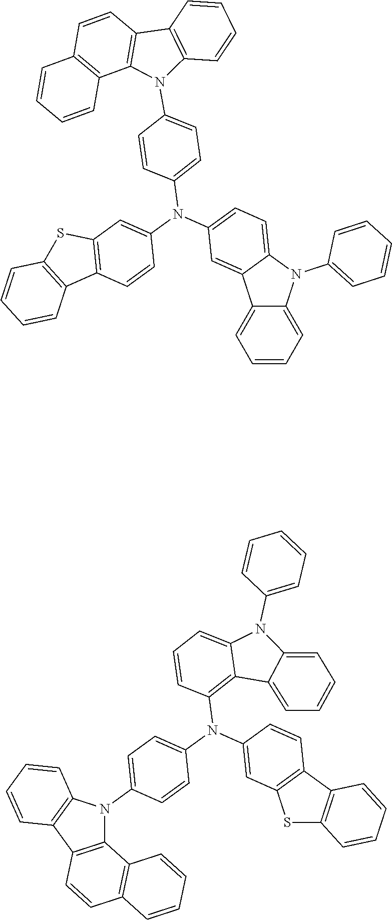 [SMILES: c1ccc(-n2c3ccccc3c3cc(N(c4ccc(-n5c6ccccc6c6ccc7ccccc7c65)cc4)c4ccc5c(c4)sc4ccccc45)ccc32)cc1 ; c1ccc(-n2c3ccccc3c3c(N(c4ccc(-n5c6ccccc6c6ccc7ccccc7c65)cc4)c4ccc5c(c4)sc4ccccc45)cccc32)cc1] 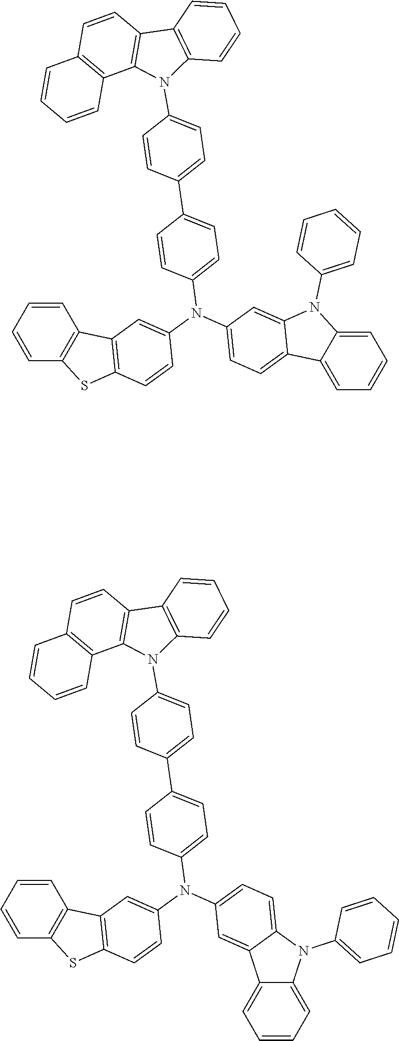 [SMILES: c1ccc(-n2c3ccccc3c3ccc(N(c4ccc(-c5ccc(-n6c7ccccc7c7ccc8ccccc8c76)cc5)cc4)c4ccc5sc6ccccc6c5c4)cc32)cc1 ; c1ccc(-n2c3ccccc3c3cc(N(c4ccc(-c5ccc(-n6c7ccccc7c7ccc8ccccc8c76)cc5)cc4)c4ccc5sc6ccccc6c5c4)ccc32)cc1] 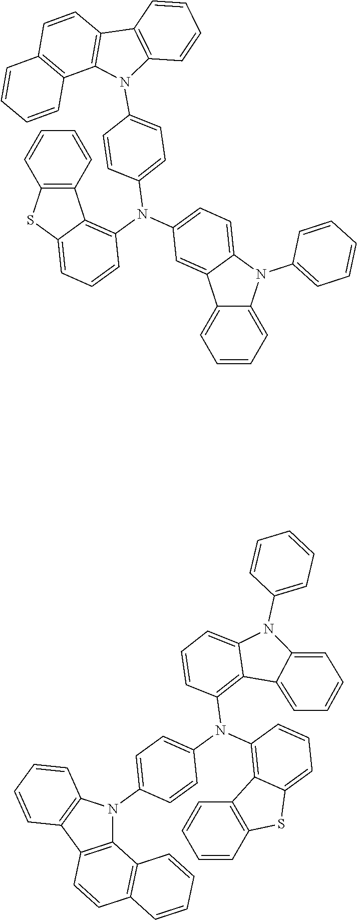 [SMILES: c1ccc(-n2c3ccccc3c3cc(N(c4ccc(-n5c6ccccc6c6ccc7ccccc7c65)cc4)c4cccc5sc6ccccc6c45)ccc32)cc1 ; c1ccc(-n2c3ccccc3c3c(N(c4ccc(-n5c6ccccc6c6ccc7ccccc7c65)cc4)c4cccc5sc6ccccc6c45)cccc32)cc1] 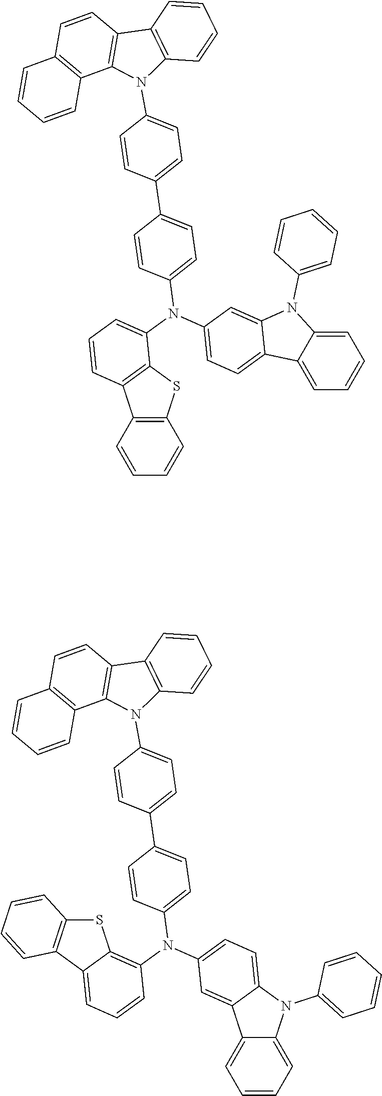 [SMILES: c1ccc(-n2c3ccccc3c3ccc(N(c4ccc(-c5ccc(-n6c7ccccc7c7ccc8ccccc8c76)cc5)cc4)c4cccc5c4sc4ccccc45)cc32)cc1 ; c1ccc(-n2c3ccccc3c3cc(N(c4ccc(-c5ccc(-n6c7ccccc7c7ccc8ccccc8c76)cc5)cc4)c4cccc5c4sc4ccccc45)ccc32)cc1] 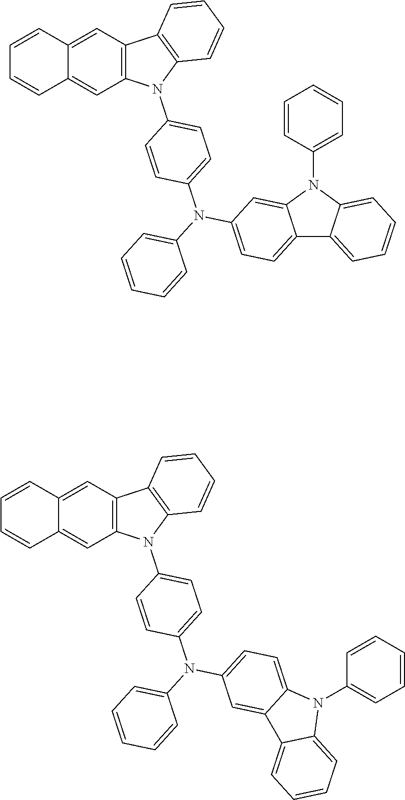 [SMILES: c1ccc(N(c2ccc(-n3c4ccccc4c4cc5ccccc5cc43)cc2)c2ccc3c4ccccc4n(-c4ccccc4)c3c2)cc1 ; c1ccc(N(c2ccc(-n3c4ccccc4c4cc5ccccc5cc43)cc2)c2ccc3c(c2)c2ccccc2n3-c2ccccc2)cc1] 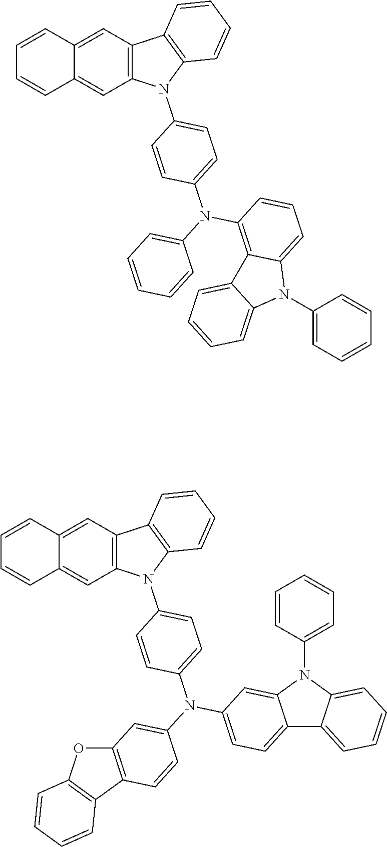 [SMILES: c1ccc(N(c2ccc(-n3c4ccccc4c4cc5ccccc5cc43)cc2)c2cccc3c2c2ccccc2n3-c2ccccc2)cc1 ; c1ccc(-n2c3ccccc3c3ccc(N(c4ccc(-n5c6ccccc6c6cc7ccccc7cc65)cc4)c4ccc5c(c4)oc4ccccc45)cc32)cc1] 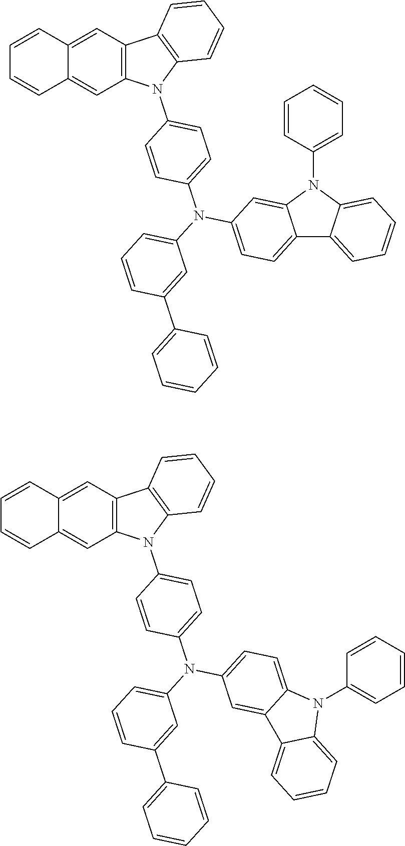 [SMILES: c1ccc(-c2cccc(N(c3ccc(-n4c5ccccc5c5cc6ccccc6cc54)cc3)c3ccc4c5ccccc5n(-c5ccccc5)c4c3)c2)cc1 ; c1ccc(-c2cccc(N(c3ccc(-n4c5ccccc5c5cc6ccccc6cc54)cc3)c3ccc4c(c3)c3ccccc3n4-c3ccccc3)c2)cc1] 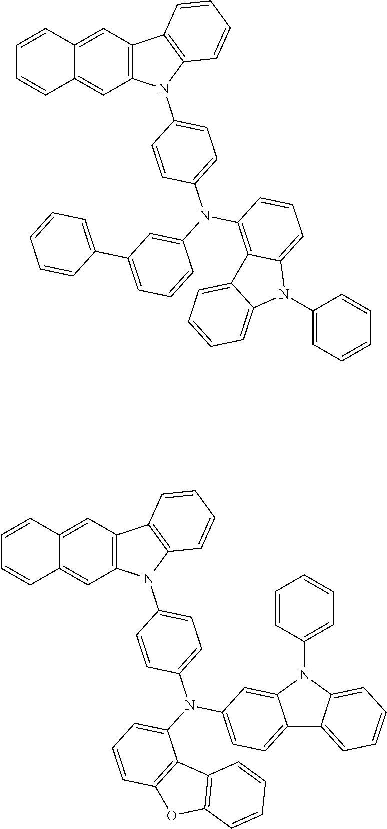 [SMILES: c1ccc(-c2cccc(N(c3ccc(-n4c5ccccc5c5cc6ccccc6cc54)cc3)c3cccc4c3c3ccccc3n4-c3ccccc3)c2)cc1 ; c1ccc(-n2c3ccccc3c3ccc(N(c4ccc(-n5c6ccccc6c6cc7ccccc7cc65)cc4)c4cccc5oc6ccccc6c45)cc32)cc1] 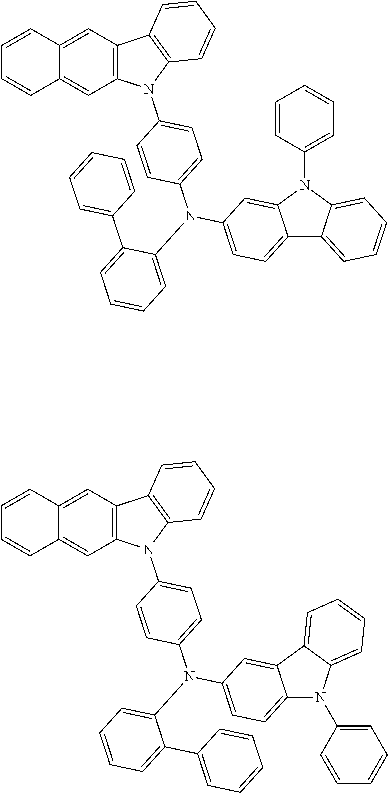 [SMILES: c1ccc(-c2ccccc2N(c2ccc(-n3c4ccccc4c4cc5ccccc5cc43)cc2)c2ccc3c4ccccc4n(-c4ccccc4)c3c2)cc1 ; c1ccc(-c2ccccc2N(c2ccc(-n3c4ccccc4c4cc5ccccc5cc43)cc2)c2ccc3c(c2)c2ccccc2n3-c2ccccc2)cc1] 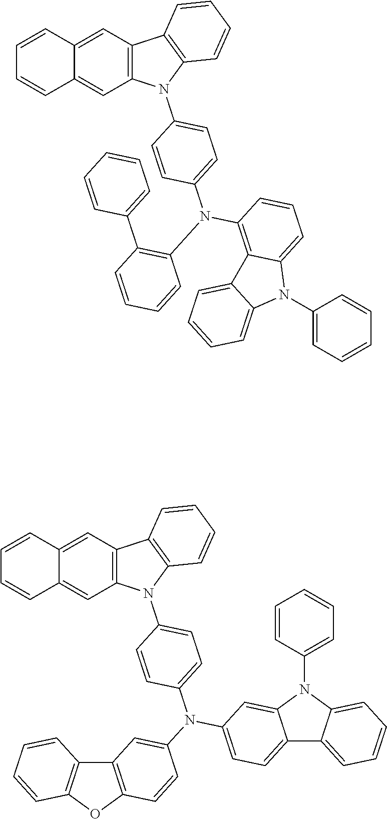 [SMILES: c1ccc(-c2ccccc2N(c2ccc(-n3c4ccccc4c4cc5ccccc5cc43)cc2)c2cccc3c2c2ccccc2n3-c2ccccc2)cc1 ; c1ccc(-n2c3ccccc3c3ccc(N(c4ccc(-n5c6ccccc6c6cc7ccccc7cc65)cc4)c4ccc5oc6ccccc6c5c4)cc32)cc1] 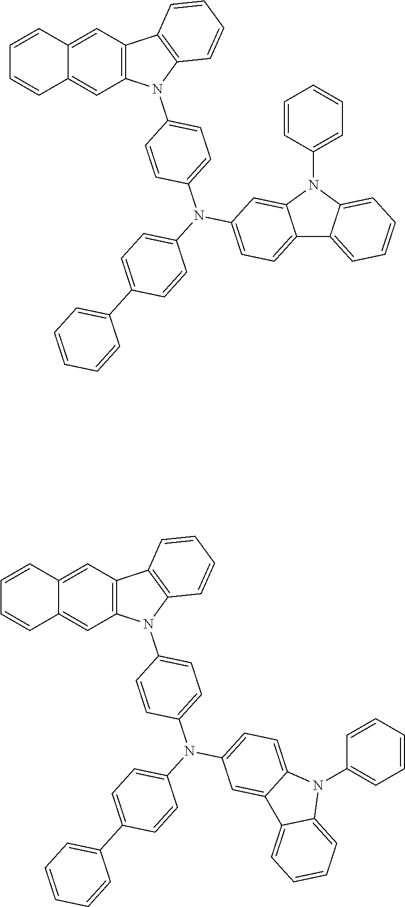 [SMILES: c1ccc(-c2ccc(N(c3ccc(-n4c5ccccc5c5cc6ccccc6cc54)cc3)c3ccc4c5ccccc5n(-c5ccccc5)c4c3)cc2)cc1 ; c1ccc(-c2ccc(N(c3ccc(-n4c5ccccc5c5cc6ccccc6cc54)cc3)c3ccc4c(c3)c3ccccc3n4-c3ccccc3)cc2)cc1] 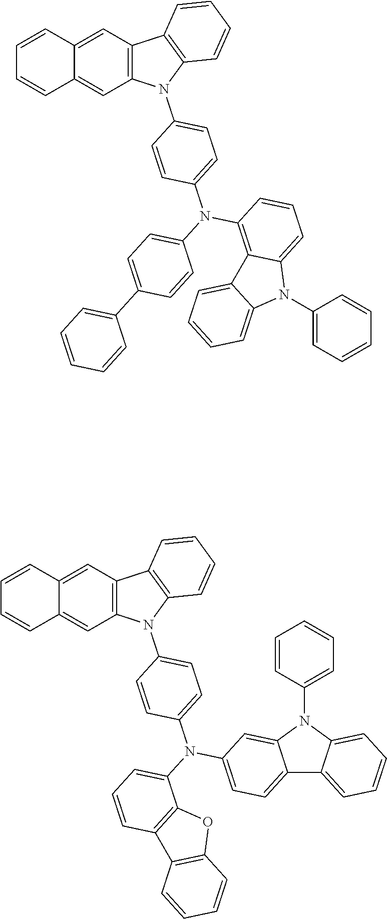 [SMILES: c1ccc(-c2ccc(N(c3ccc(-n4c5ccccc5c5cc6ccccc6cc54)cc3)c3cccc4c3c3ccccc3n4-c3ccccc3)cc2)cc1 ; c1ccc(-n2c3ccccc3c3ccc(N(c4ccc(-n5c6ccccc6c6cc7ccccc7cc65)cc4)c4cccc5c4oc4ccccc45)cc32)cc1] 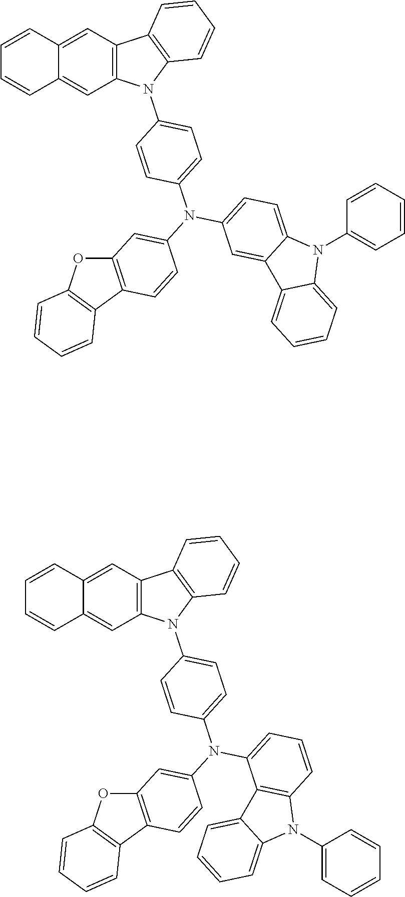 [SMILES: c1ccc(-n2c3ccccc3c3cc(N(c4ccc(-n5c6ccccc6c6cc7ccccc7cc65)cc4)c4ccc5c(c4)oc4ccccc45)ccc32)cc1 ; c1ccc(-n2c3ccccc3c3c(N(c4ccc(-n5c6ccccc6c6cc7ccccc7cc65)cc4)c4ccc5c(c4)oc4ccccc45)cccc32)cc1] 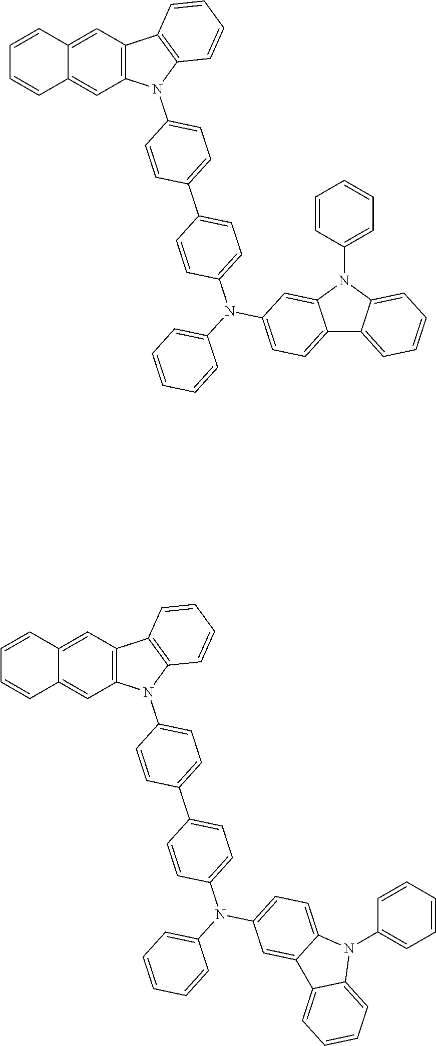 [SMILES: c1ccc(N(c2ccc(-c3ccc(-n4c5ccccc5c5cc6ccccc6cc54)cc3)cc2)c2ccc3c4ccccc4n(-c4ccccc4)c3c2)cc1 ; c1ccc(N(c2ccc(-c3ccc(-n4c5ccccc5c5cc6ccccc6cc54)cc3)cc2)c2ccc3c(c2)c2ccccc2n3-c2ccccc2)cc1] 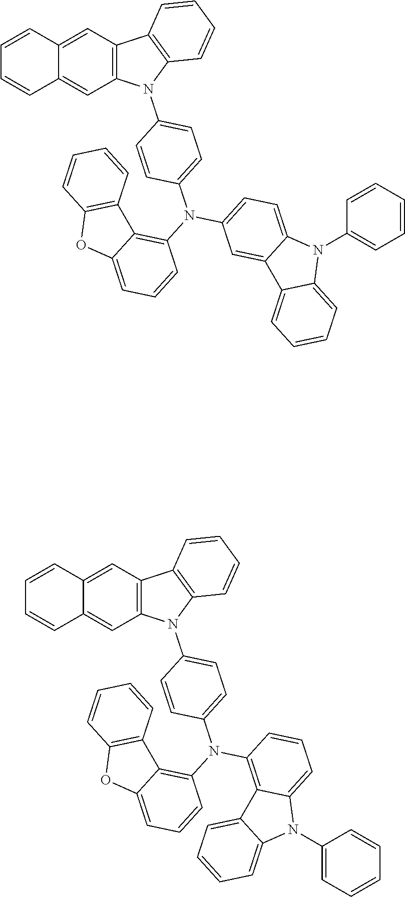 [SMILES: c1ccc(-n2c3ccccc3c3cc(N(c4ccc(-n5c6ccccc6c6cc7ccccc7cc65)cc4)c4cccc5oc6ccccc6c45)ccc32)cc1 ; c1ccc(-n2c3ccccc3c3c(N(c4ccc(-n5c6ccccc6c6cc7ccccc7cc65)cc4)c4cccc5oc6ccccc6c45)cccc32)cc1] 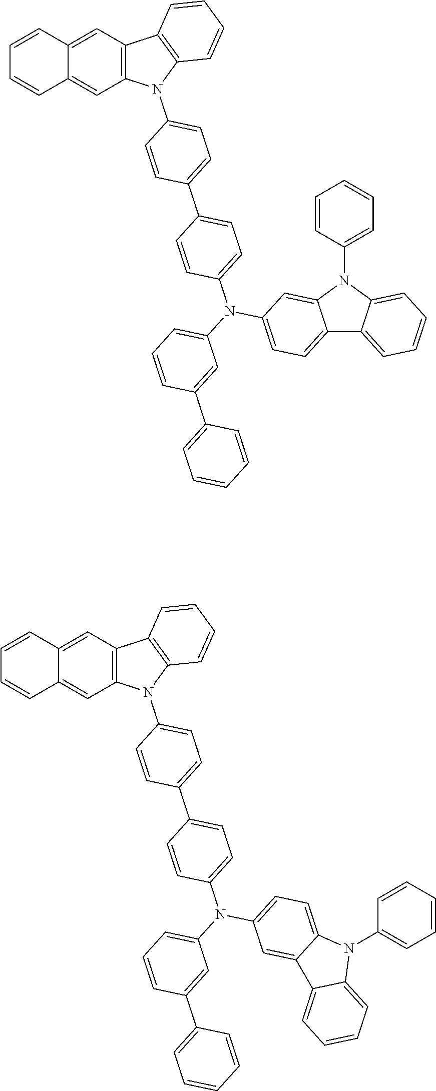 [SMILES: c1ccc(-c2cccc(N(c3ccc(-c4ccc(-n5c6ccccc6c6cc7ccccc7cc65)cc4)cc3)c3ccc4c5ccccc5n(-c5ccccc5)c4c3)c2)cc1 ; c1ccc(-c2cccc(N(c3ccc(-c4ccc(-n5c6ccccc6c6cc7ccccc7cc65)cc4)cc3)c3ccc4c(c3)c3ccccc3n4-c3ccccc3)c2)cc1] 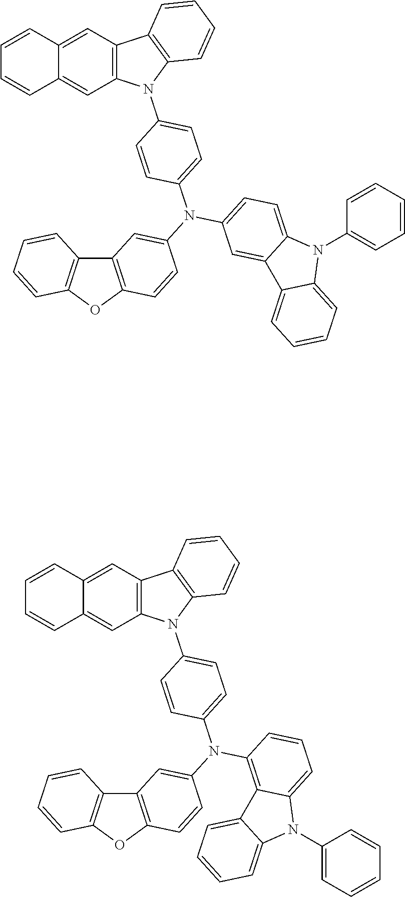 [SMILES: c1ccc(-n2c3ccccc3c3cc(N(c4ccc(-n5c6ccccc6c6cc7ccccc7cc65)cc4)c4ccc5oc6ccccc6c5c4)ccc32)cc1 ; c1ccc(-n2c3ccccc3c3c(N(c4ccc(-n5c6ccccc6c6cc7ccccc7cc65)cc4)c4ccc5oc6ccccc6c5c4)cccc32)cc1] 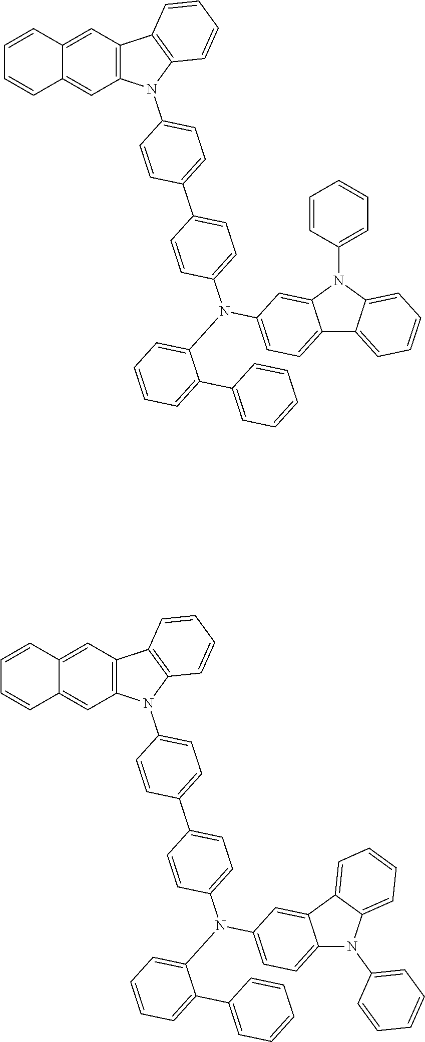 [SMILES: c1ccc(-c2ccccc2N(c2ccc(-c3ccc(-n4c5ccccc5c5cc6ccccc6cc54)cc3)cc2)c2ccc3c4ccccc4n(-c4ccccc4)c3c2)cc1 ; c1ccc(-c2ccccc2N(c2ccc(-c3ccc(-n4c5ccccc5c5cc6ccccc6cc54)cc3)cc2)c2ccc3c(c2)c2ccccc2n3-c2ccccc2)cc1] 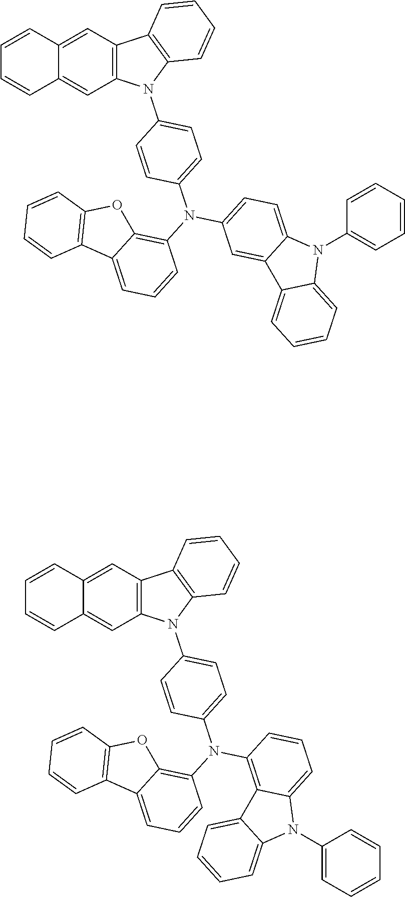 [SMILES: c1ccc(-n2c3ccccc3c3cc(N(c4ccc(-n5c6ccccc6c6cc7ccccc7cc65)cc4)c4cccc5c4oc4ccccc45)ccc32)cc1 ; c1ccc(-n2c3ccccc3c3c(N(c4ccc(-n5c6ccccc6c6cc7ccccc7cc65)cc4)c4cccc5c4oc4ccccc45)cccc32)cc1] 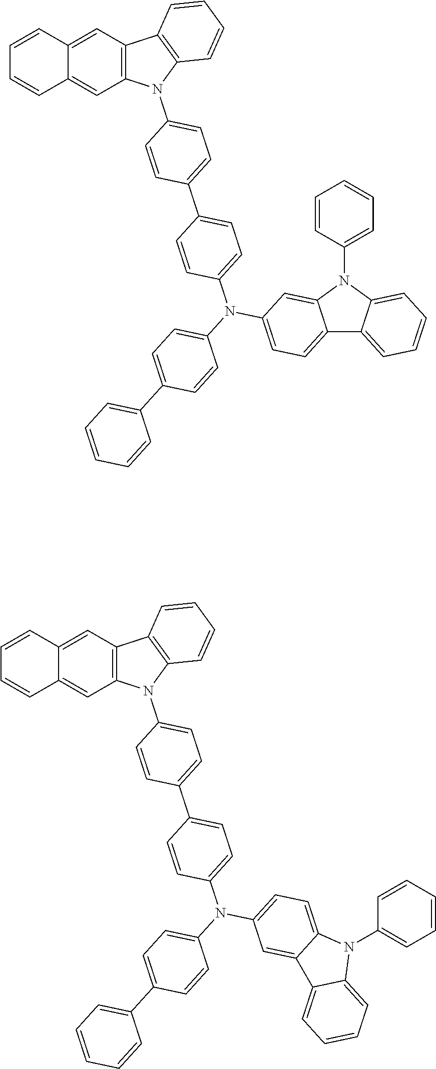 [SMILES: c1ccc(-c2ccc(N(c3ccc(-c4ccc(-n5c6ccccc6c6cc7ccccc7cc65)cc4)cc3)c3ccc4c5ccccc5n(-c5ccccc5)c4c3)cc2)cc1 ; c1ccc(-c2ccc(N(c3ccc(-c4ccc(-n5c6ccccc6c6cc7ccccc7cc65)cc4)cc3)c3ccc4c(c3)c3ccccc3n4-c3ccccc3)cc2)cc1] 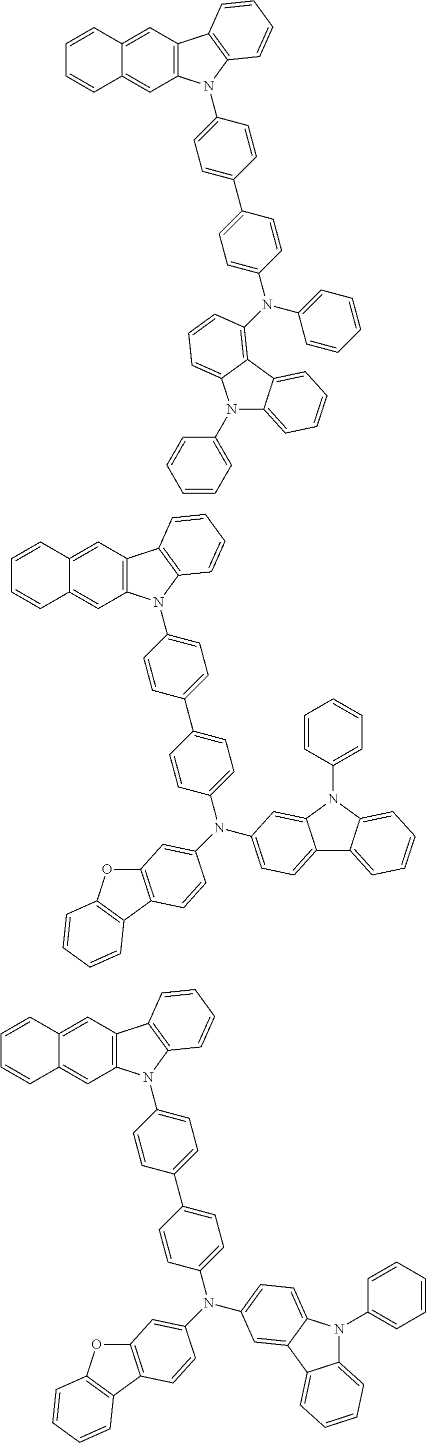 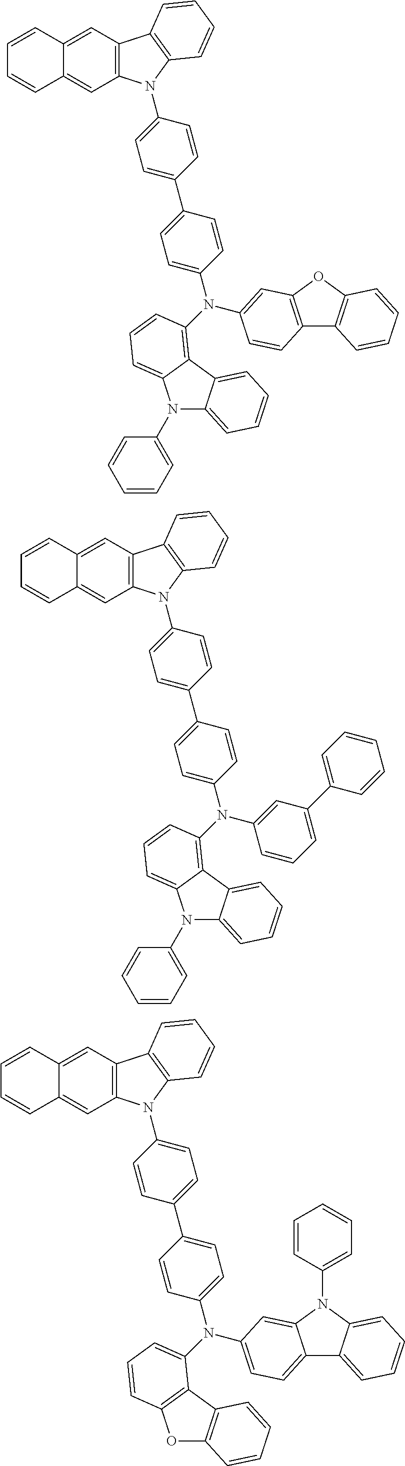 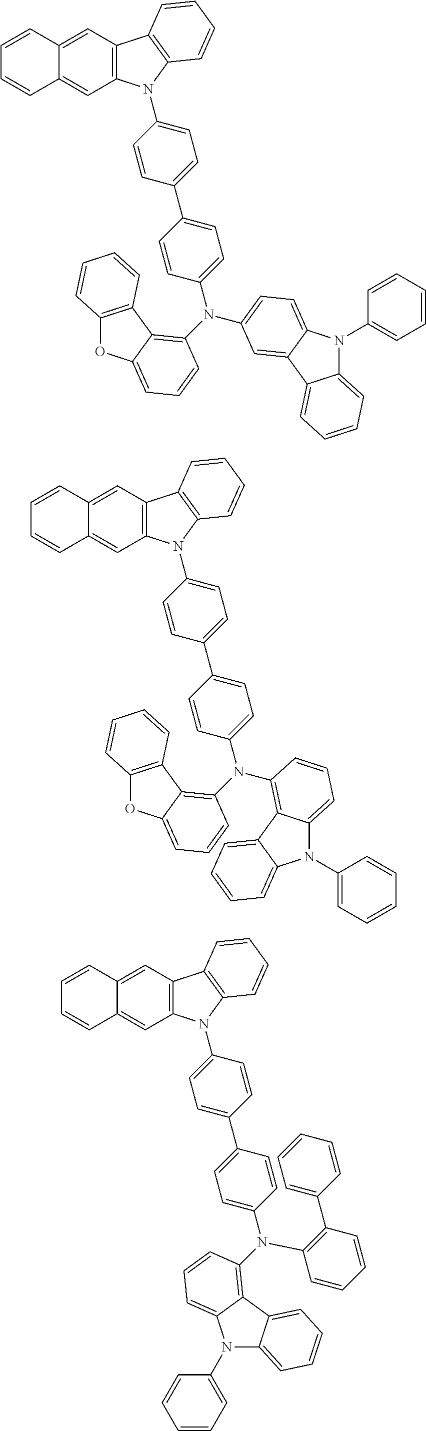 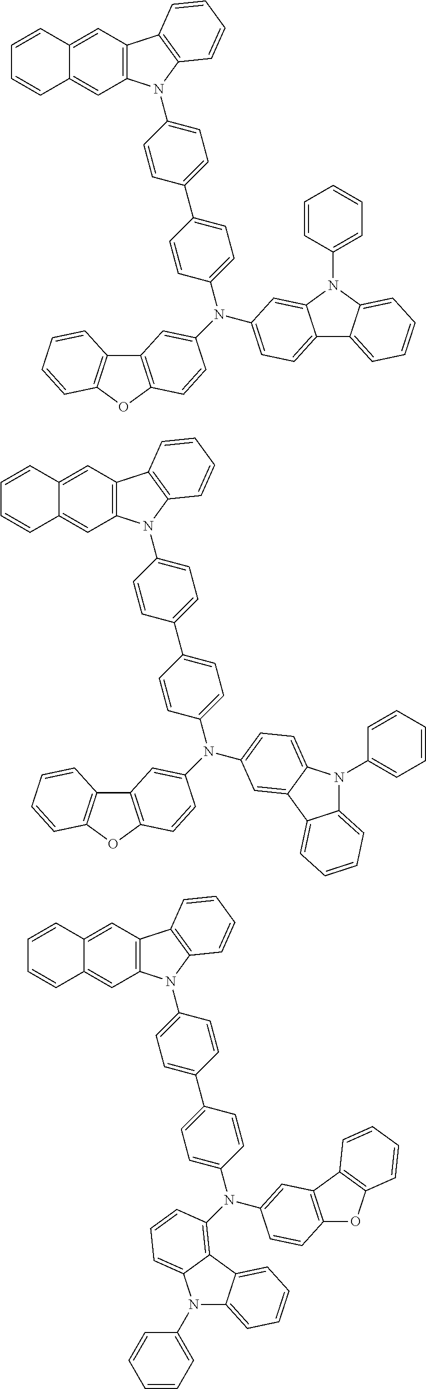 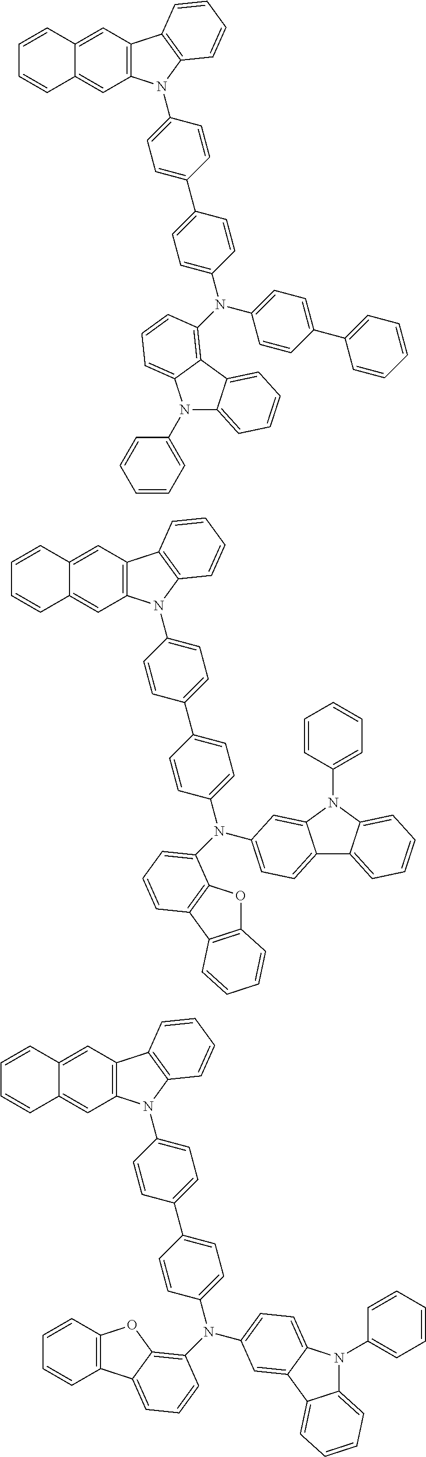 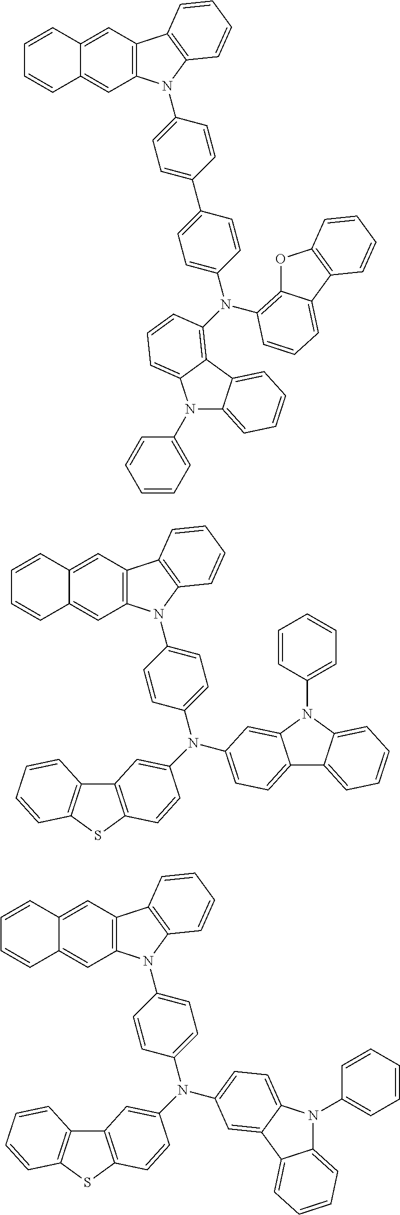 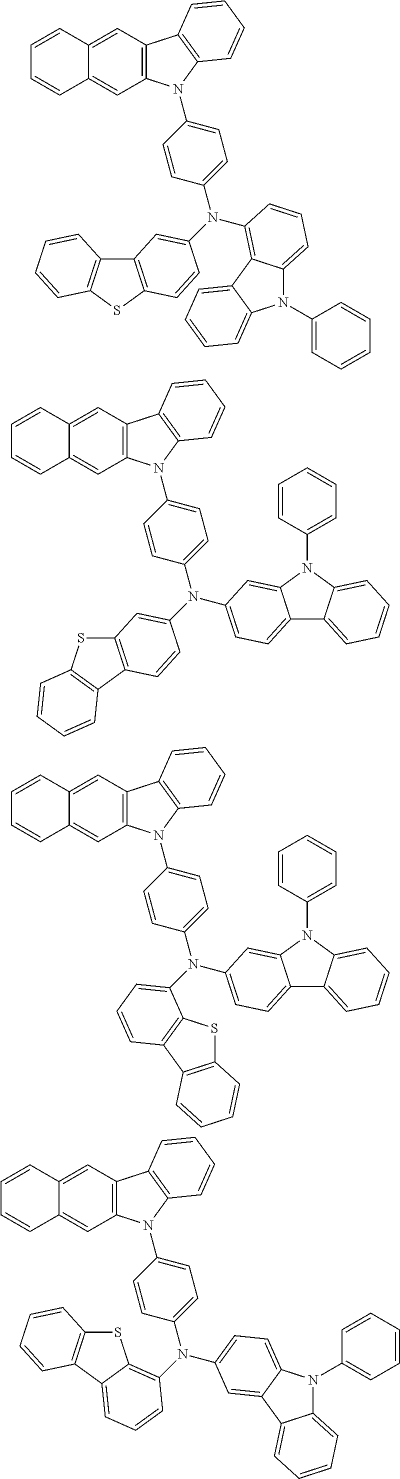 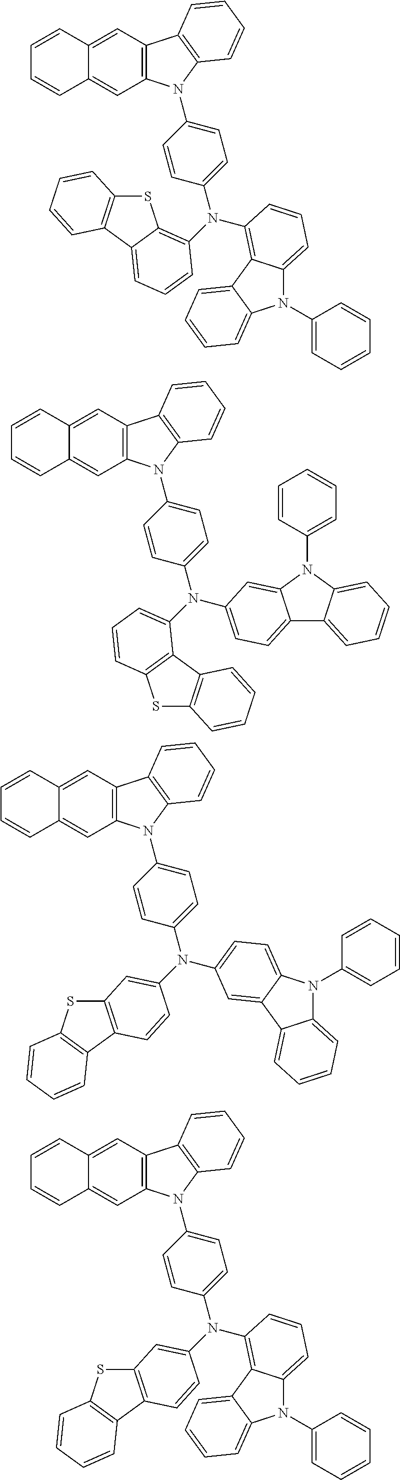 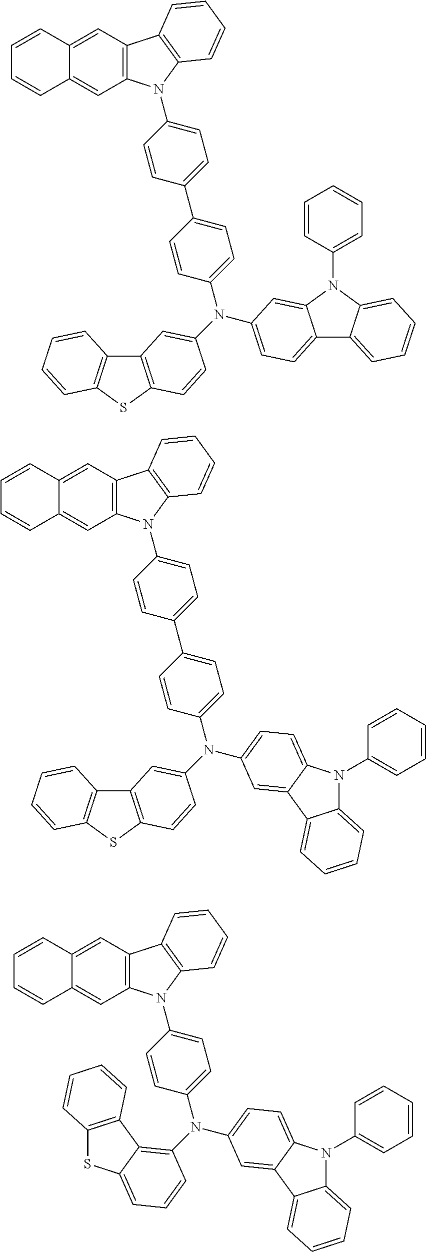 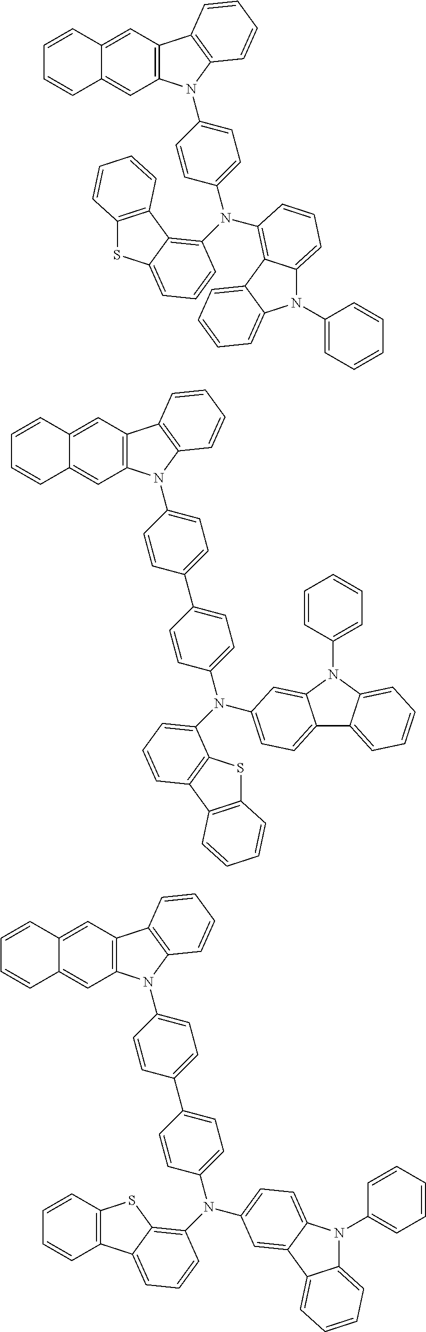 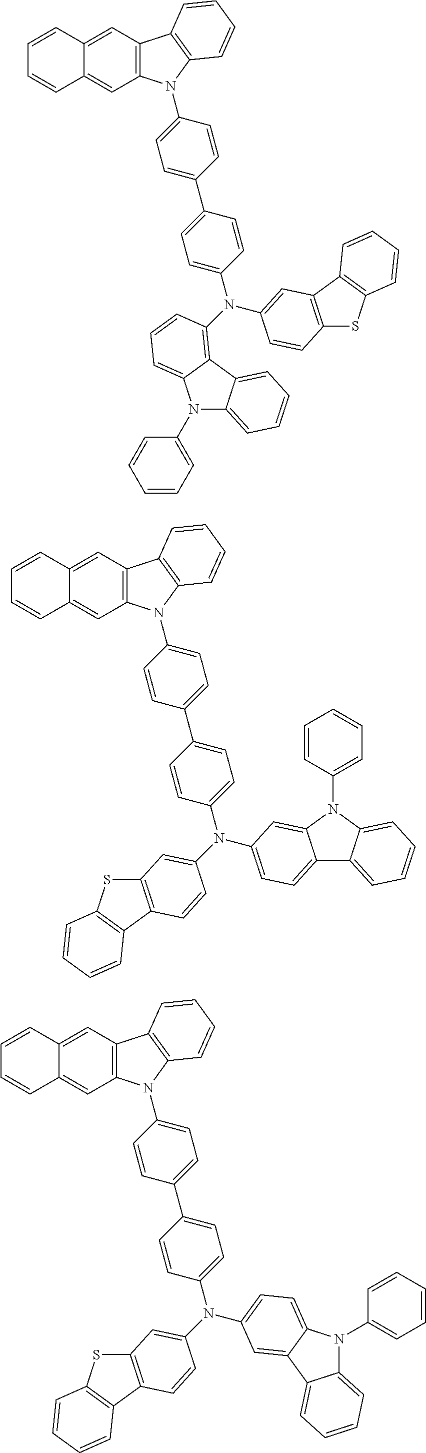 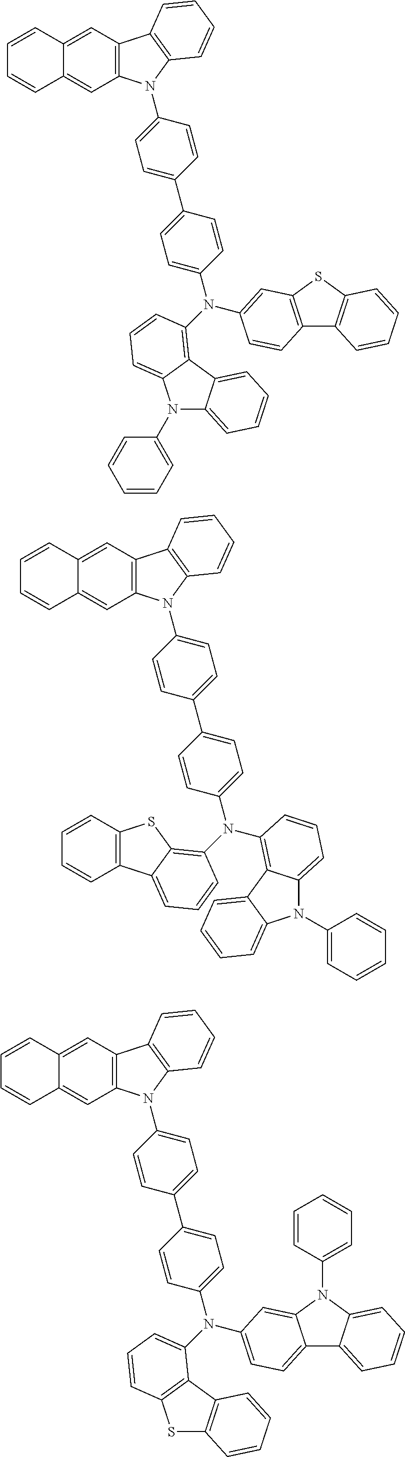 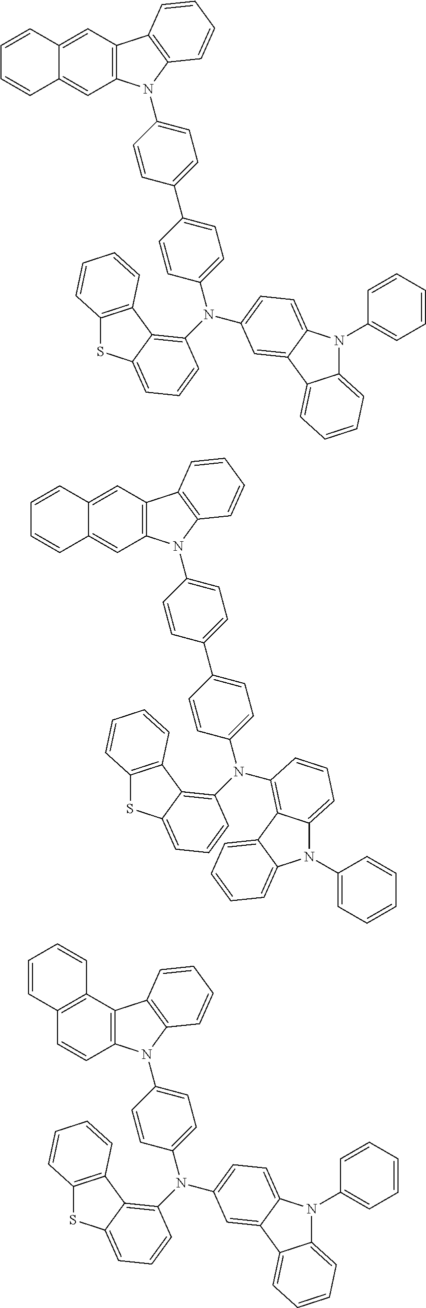 [SMILES: c1ccc(-n2c3ccccc3c3cc(N(c4ccc(-c5ccc(-n6c7ccccc7c7cc8ccccc8cc76)cc5)cc4)c4cccc5sc6ccccc6c45)ccc32)cc1 ; c1ccc(-n2c3ccccc3c3c(N(c4ccc(-c5ccc(-n6c7ccccc7c7cc8ccccc8cc76)cc5)cc4)c4cccc5sc6ccccc6c45)cccc32)cc1 ; c1ccc(-n2c3ccccc3c3cc(N(c4ccc(-n5c6ccccc6c6c7ccccc7ccc65)cc4)c4cccc5sc6ccccc6c45)ccc32)cc1] 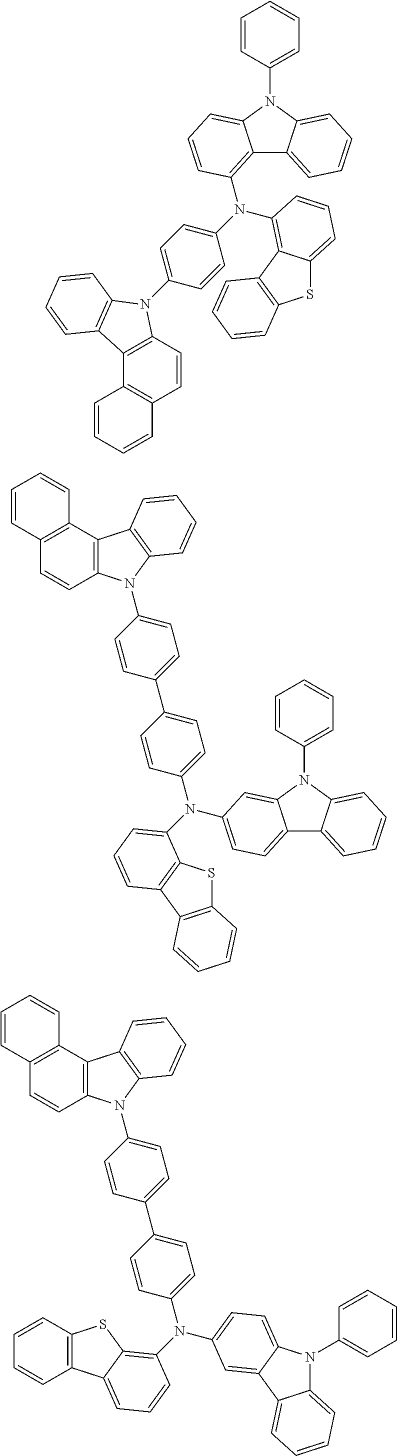 [SMILES: c1ccc(-n2c3ccccc3c3c(N(c4ccc(-n5c6ccccc6c6c7ccccc7ccc65)cc4)c4cccc5sc6ccccc6c45)cccc32)cc1 ; c1ccc(-n2c3ccccc3c3ccc(N(c4ccc(-c5ccc(-n6c7ccccc7c7c8ccccc8ccc76)cc5)cc4)c4cccc5c4sc4ccccc45)cc32)cc1 ; c1ccc(-n2c3ccccc3c3cc(N(c4ccc(-c5ccc(-n6c7ccccc7c7c8ccccc8ccc76)cc5)cc4)c4cccc5c4sc4ccccc45)ccc32)cc1] 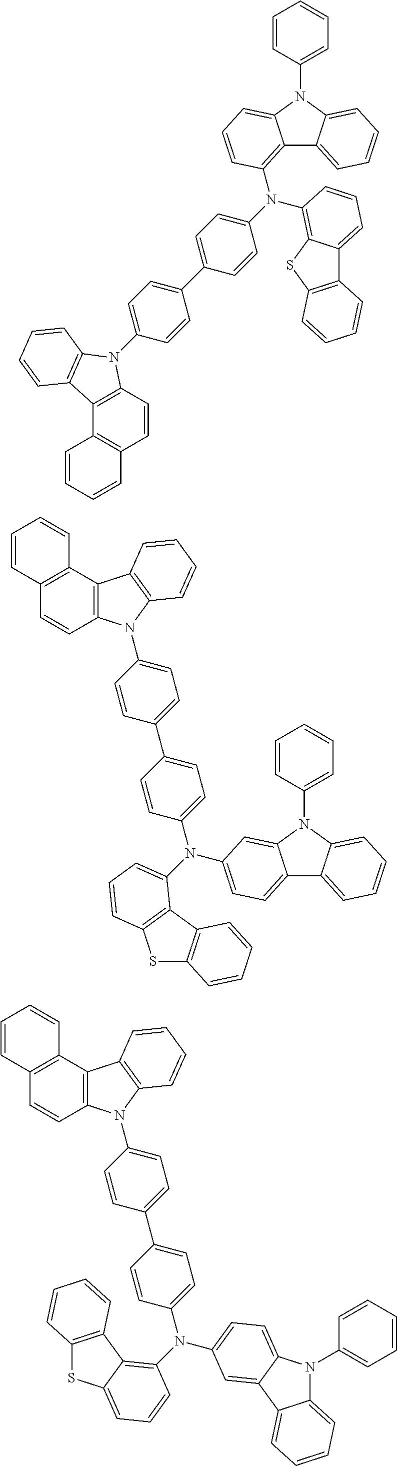 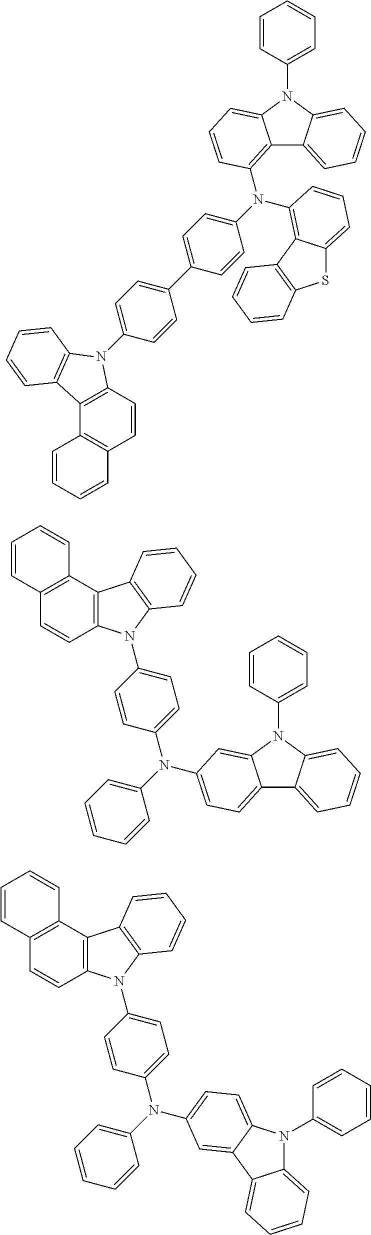 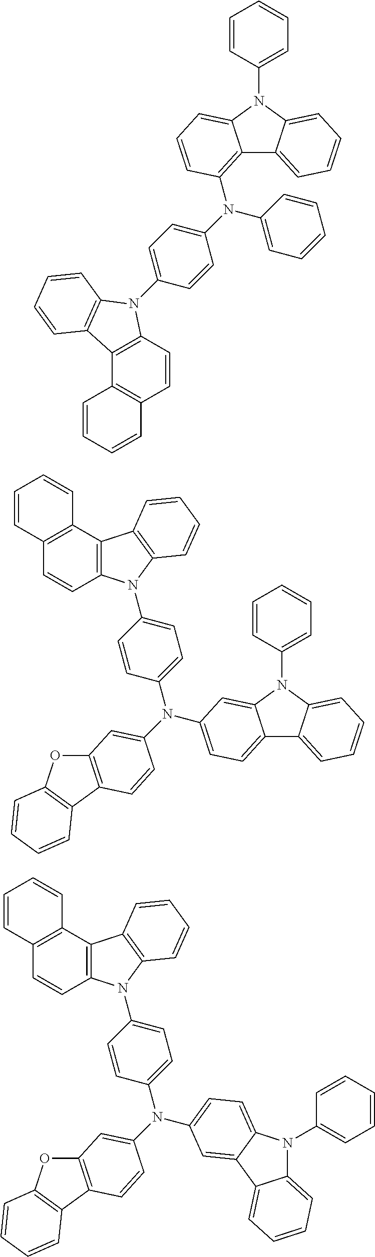 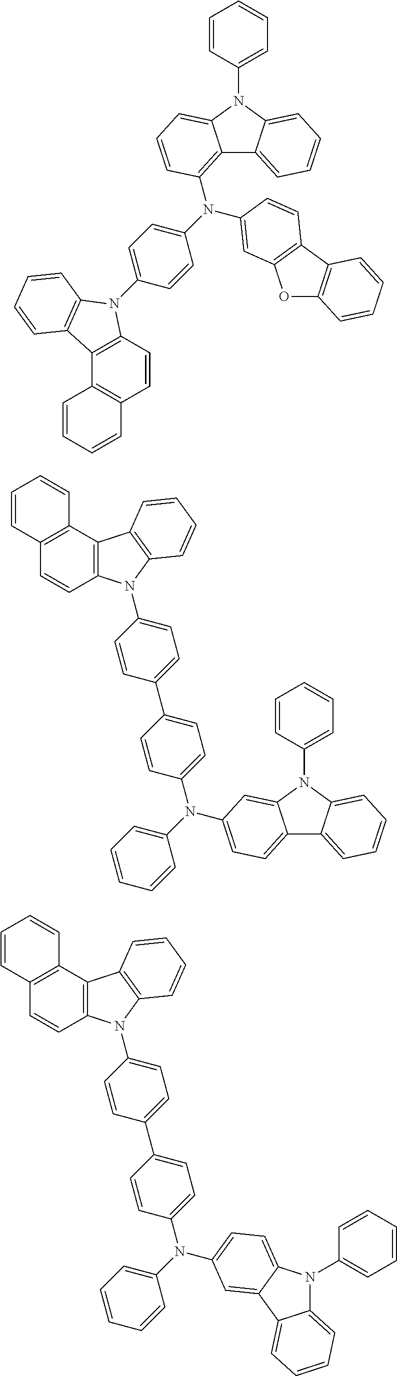 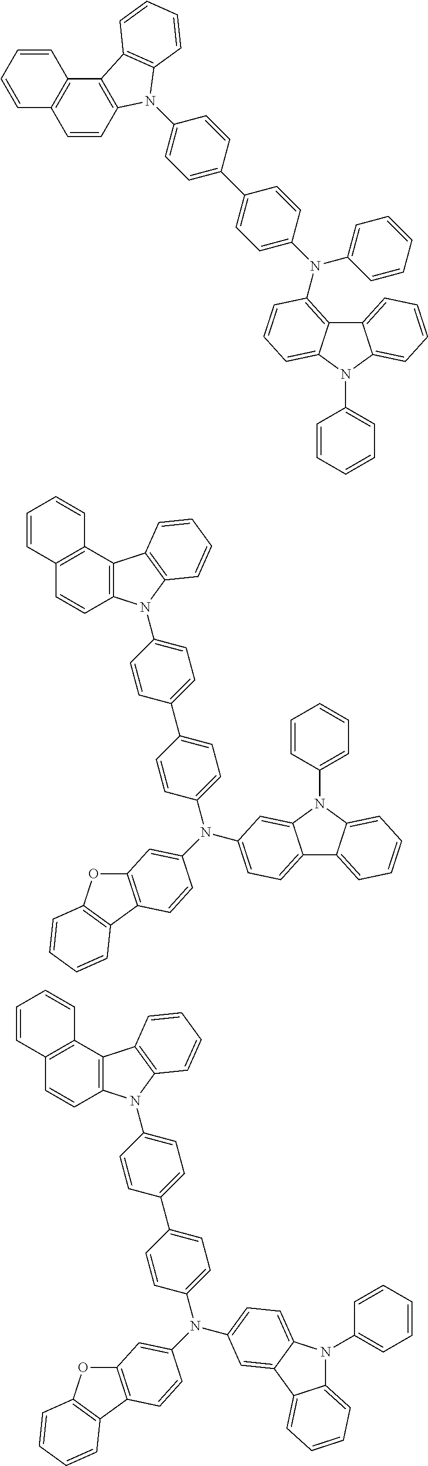 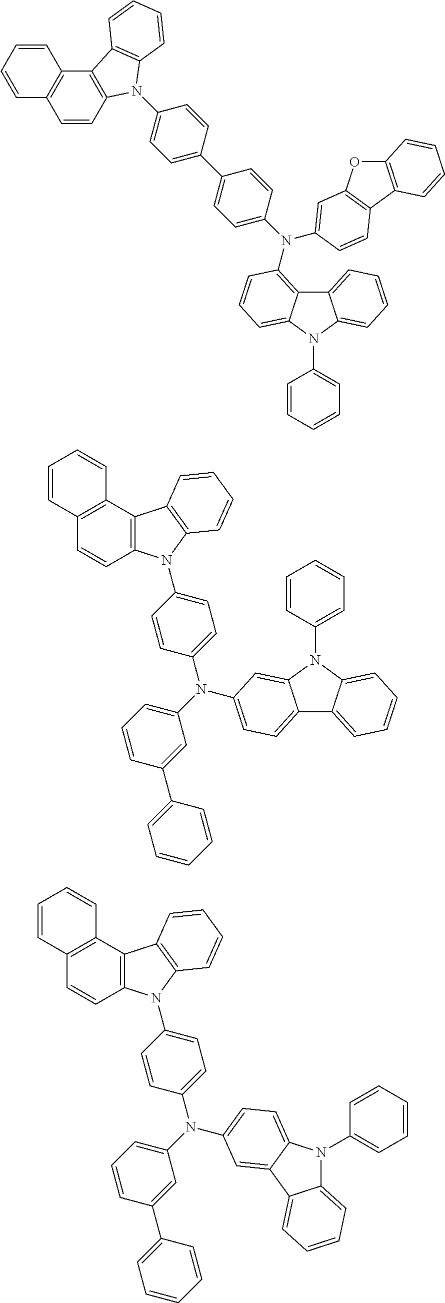 [SMILES: c1ccc(-n2c3ccccc3c3c(N(c4ccc(-c5ccc(-n6c7ccccc7c7c8ccccc8ccc76)cc5)cc4)c4ccc5c(c4)oc4ccccc45)cccc32)cc1 ; c1ccc(-c2cccc(N(c3ccc(-n4c5ccccc5c5c6ccccc6ccc54)cc3)c3ccc4c5ccccc5n(-c5ccccc5)c4c3)c2)cc1 ; c1ccc(-c2cccc(N(c3ccc(-n4c5ccccc5c5c6ccccc6ccc54)cc3)c3ccc4c(c3)c3ccccc3n4-c3ccccc3)c2)cc1] 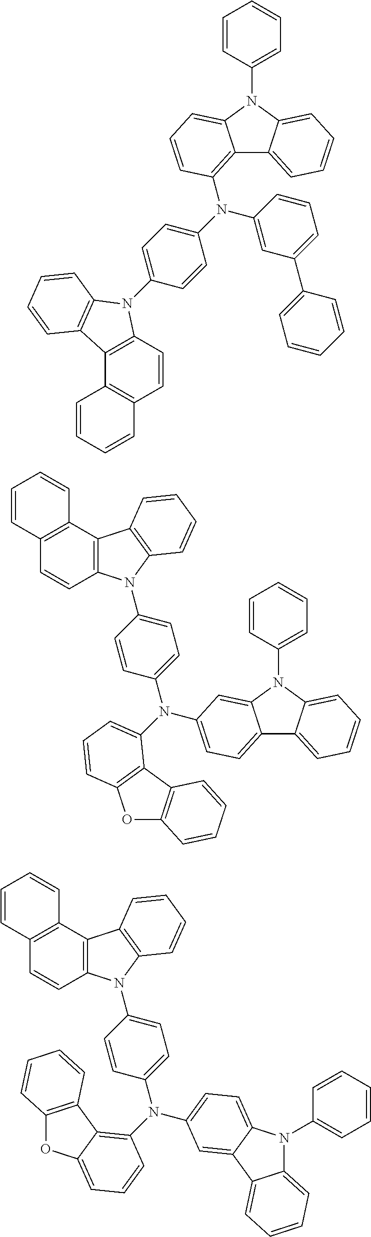 [SMILES: c1ccc(-c2cccc(N(c3ccc(-n4c5ccccc5c5c6ccccc6ccc54)cc3)c3cccc4c3c3ccccc3n4-c3ccccc3)c2)cc1 ; c1ccc(-n2c3ccccc3c3ccc(N(c4ccc(-n5c6ccccc6c6c7ccccc7ccc65)cc4)c4cccc5oc6ccccc6c45)cc32)cc1 ; c1ccc(-n2c3ccccc3c3cc(N(c4ccc(-n5c6ccccc6c6c7ccccc7ccc65)cc4)c4cccc5oc6ccccc6c45)ccc32)cc1] 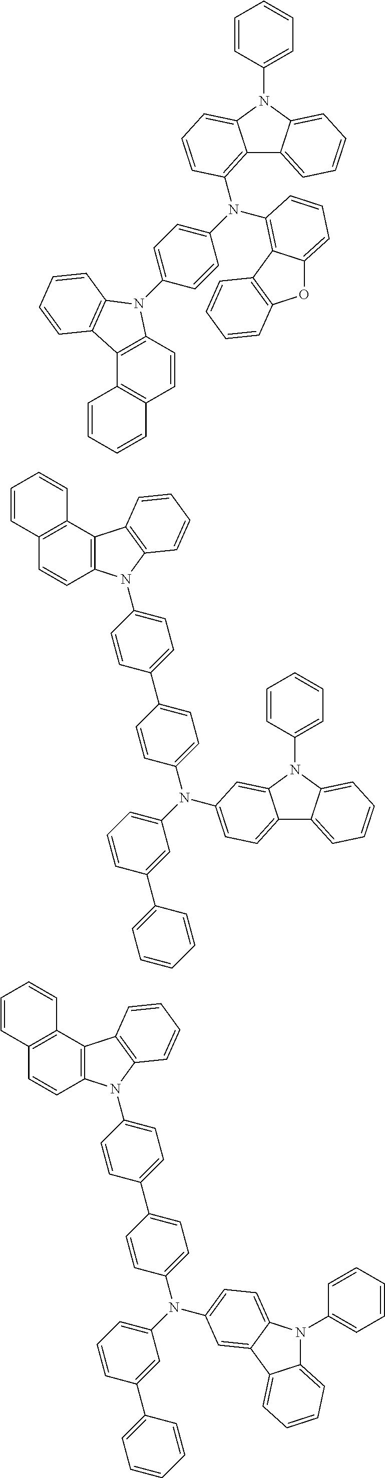 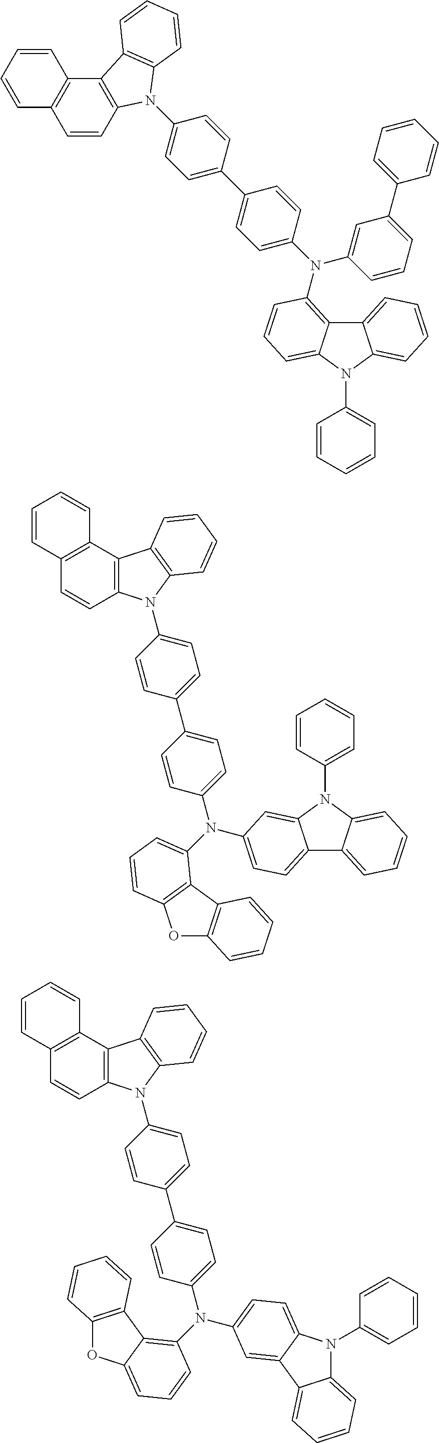 [SMILES: c1ccc(-c2cccc(N(c3ccc(-c4ccc(-n5c6ccccc6c6c7ccccc7ccc65)cc4)cc3)c3cccc4c3c3ccccc3n4-c3ccccc3)c2)cc1 ; c1ccc(-n2c3ccccc3c3ccc(N(c4ccc(-c5ccc(-n6c7ccccc7c7c8ccccc8ccc76)cc5)cc4)c4cccc5oc6ccccc6c45)cc32)cc1 ; c1ccc(-n2c3ccccc3c3cc(N(c4ccc(-c5ccc(-n6c7ccccc7c7c8ccccc8ccc76)cc5)cc4)c4cccc5oc6ccccc6c45)ccc32)cc1] 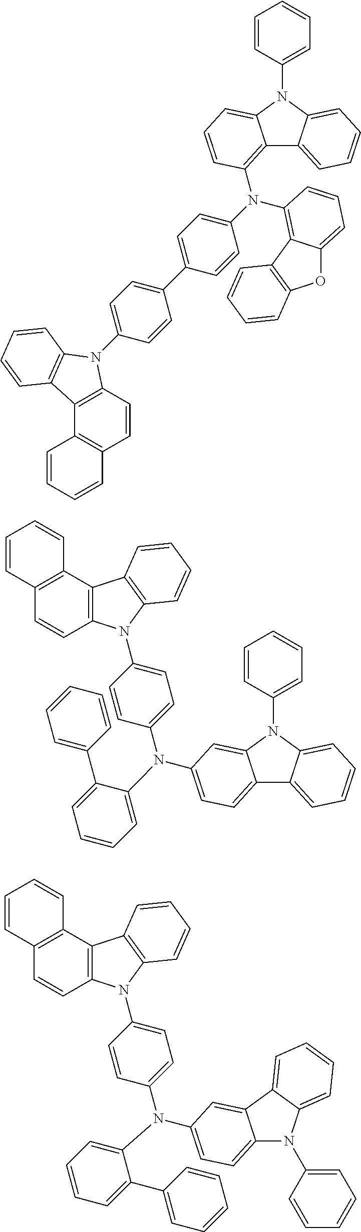 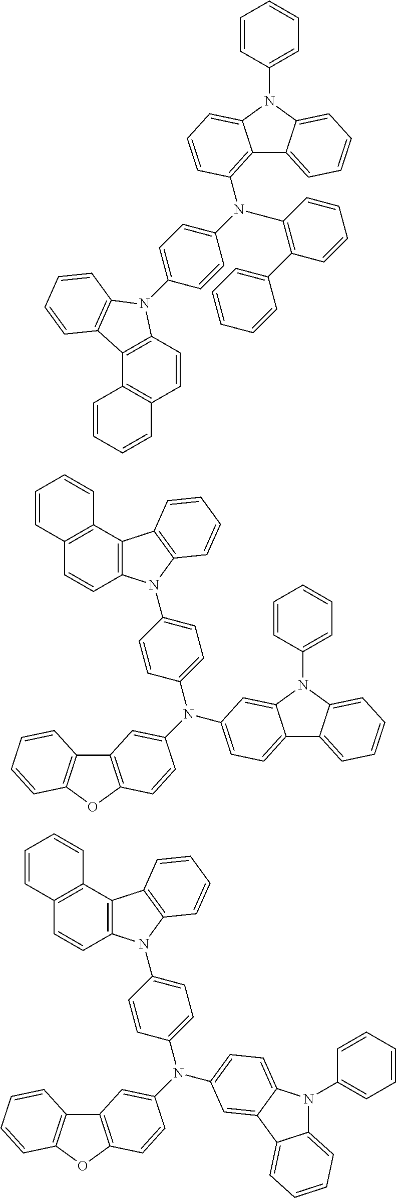 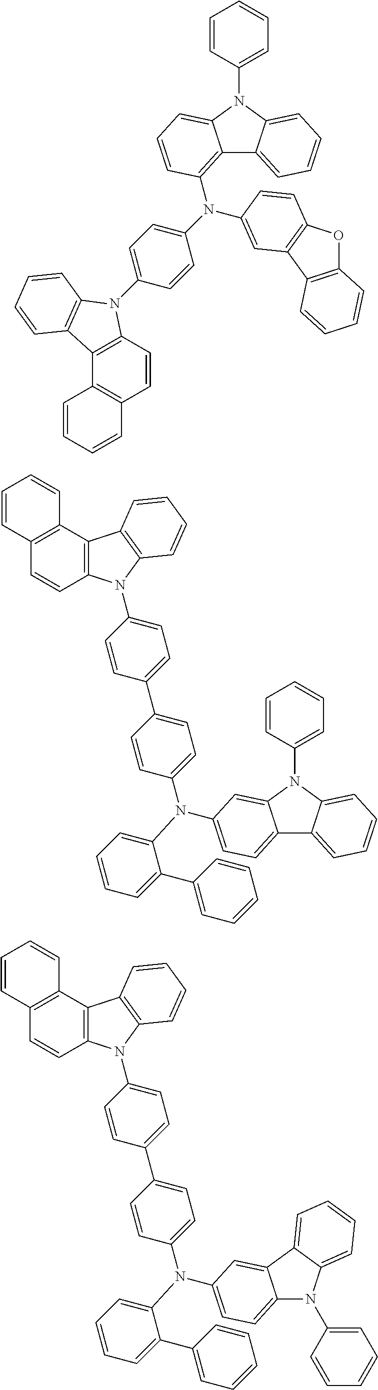 [SMILES: c1ccc(-n2c3ccccc3c3c(N(c4ccc(-n5c6ccccc6c6c7ccccc7ccc65)cc4)c4ccc5oc6ccccc6c5c4)cccc32)cc1 ; c1ccc(-c2ccccc2N(c2ccc(-c3ccc(-n4c5ccccc5c5c6ccccc6ccc54)cc3)cc2)c2ccc3c4ccccc4n(-c4ccccc4)c3c2)cc1 ; c1ccc(-c2ccccc2N(c2ccc(-c3ccc(-n4c5ccccc5c5c6ccccc6ccc54)cc3)cc2)c2ccc3c(c2)c2ccccc2n3-c2ccccc2)cc1] 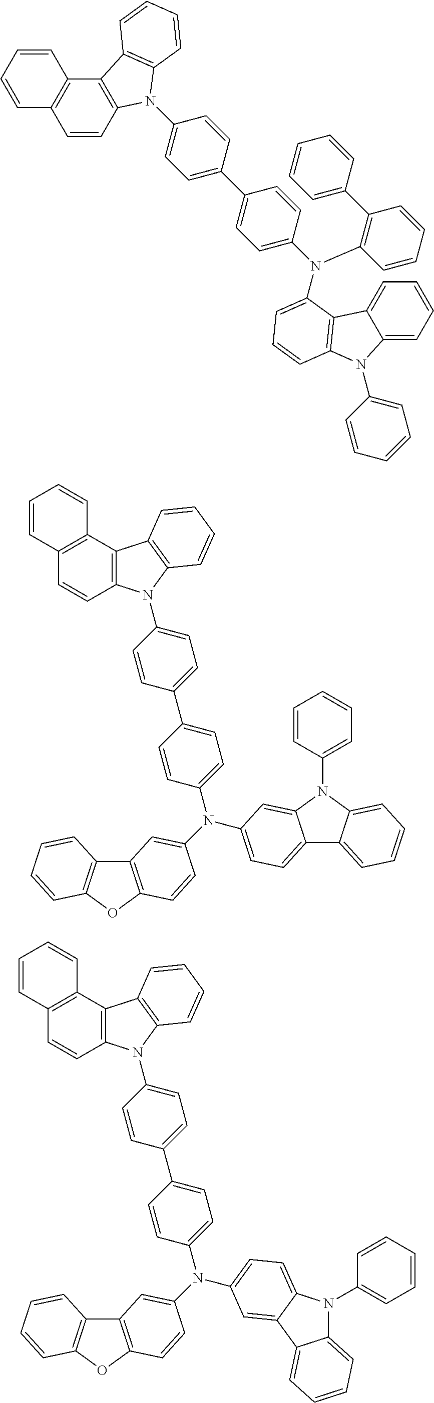 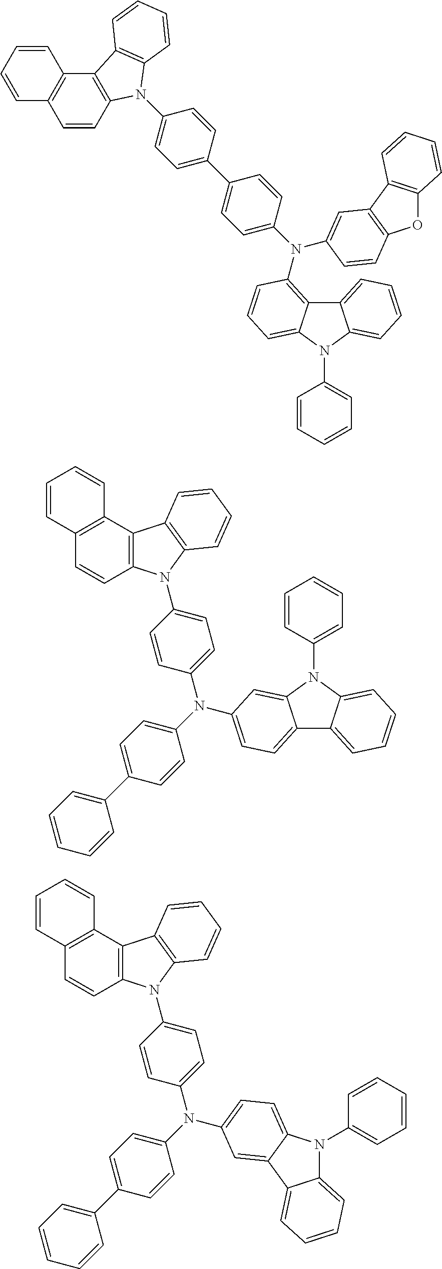 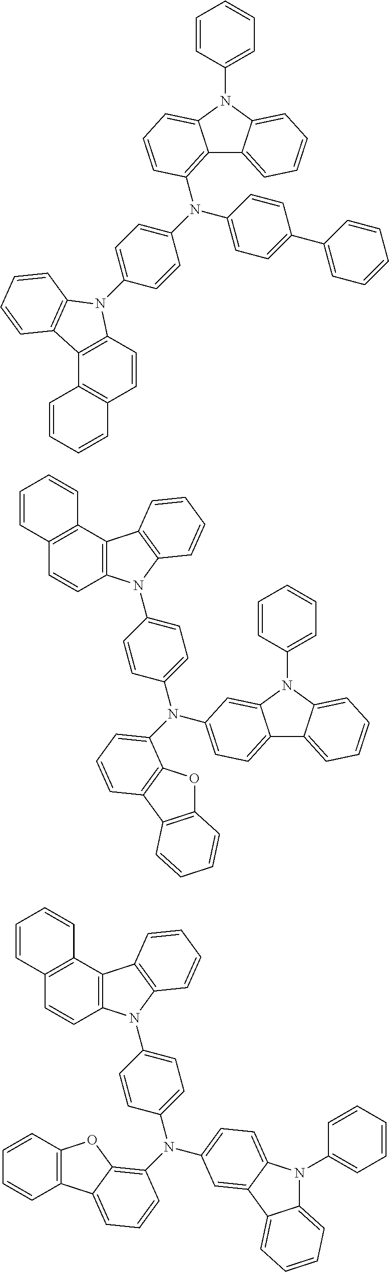 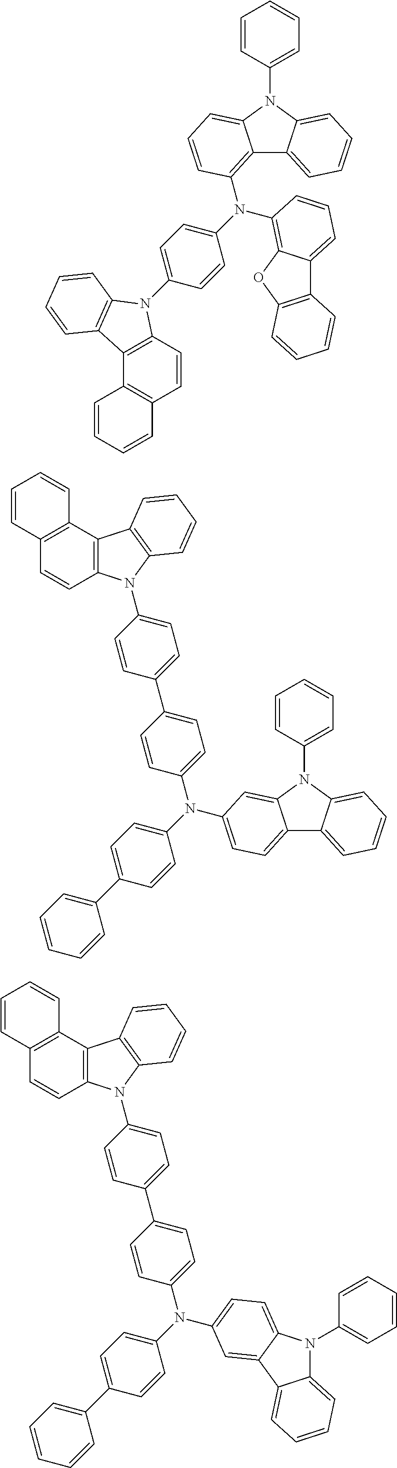 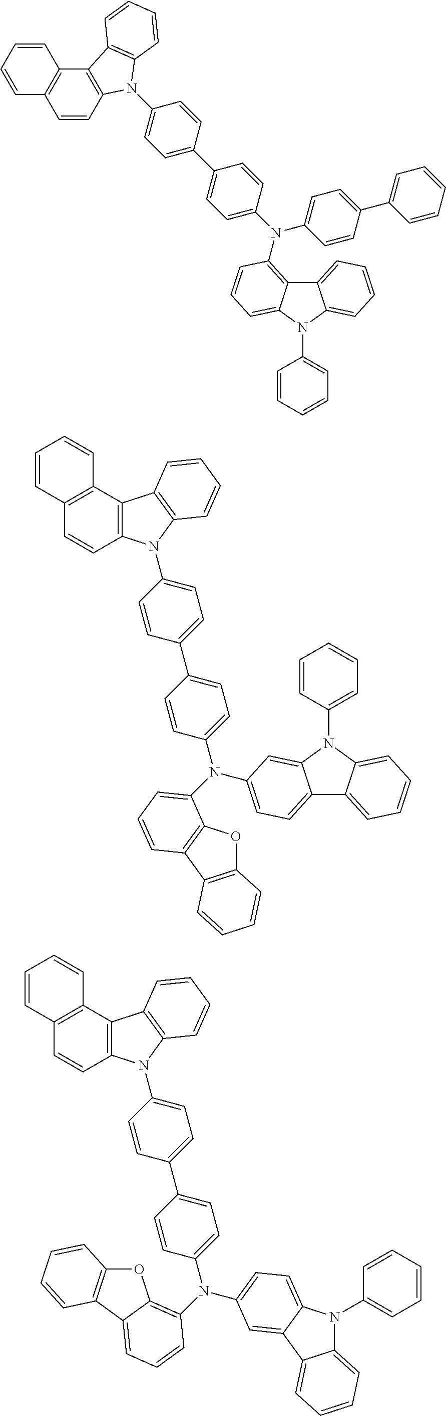 [SMILES: c1ccc(-c2ccc(N(c3ccc(-c4ccc(-n5c6ccccc6c6c7ccccc7ccc65)cc4)cc3)c3cccc4c3c3ccccc3n4-c3ccccc3)cc2)cc1 ; c1ccc(-n2c3ccccc3c3ccc(N(c4ccc(-c5ccc(-n6c7ccccc7c7c8ccccc8ccc76)cc5)cc4)c4cccc5c4oc4ccccc45)cc32)cc1 ; c1ccc(-n2c3ccccc3c3cc(N(c4ccc(-c5ccc(-n6c7ccccc7c7c8ccccc8ccc76)cc5)cc4)c4cccc5c4oc4ccccc45)ccc32)cc1] 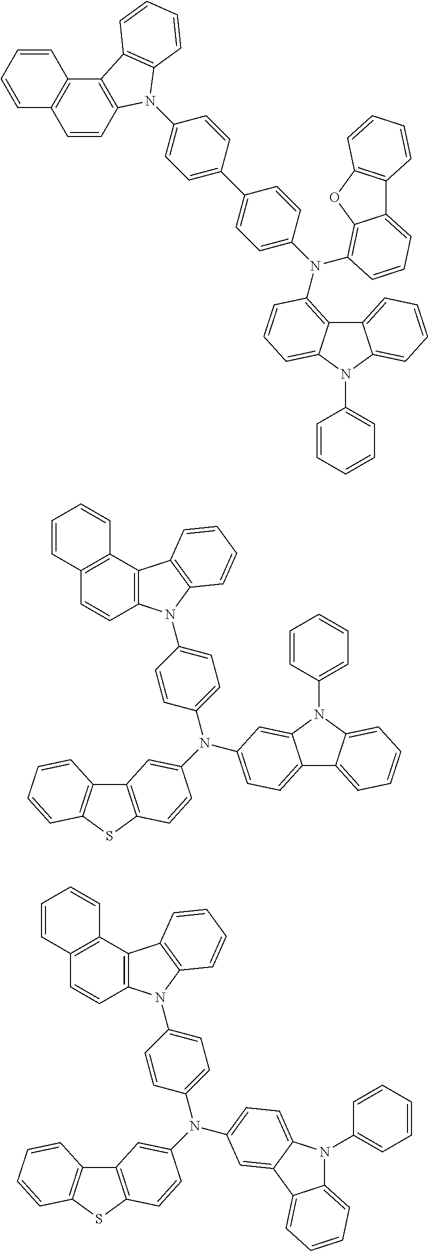 [SMILES: c1ccc(-n2c3ccccc3c3c(N(c4ccc(-c5ccc(-n6c7ccccc7c7c8ccccc8ccc76)cc5)cc4)c4cccc5c4oc4ccccc45)cccc32)cc1 ; c1ccc(-n2c3ccccc3c3ccc(N(c4ccc(-n5c6ccccc6c6c7ccccc7ccc65)cc4)c4ccc5sc6ccccc6c5c4)cc32)cc1 ; c1ccc(-n2c3ccccc3c3cc(N(c4ccc(-n5c6ccccc6c6c7ccccc7ccc65)cc4)c4ccc5sc6ccccc6c5c4)ccc32)cc1] 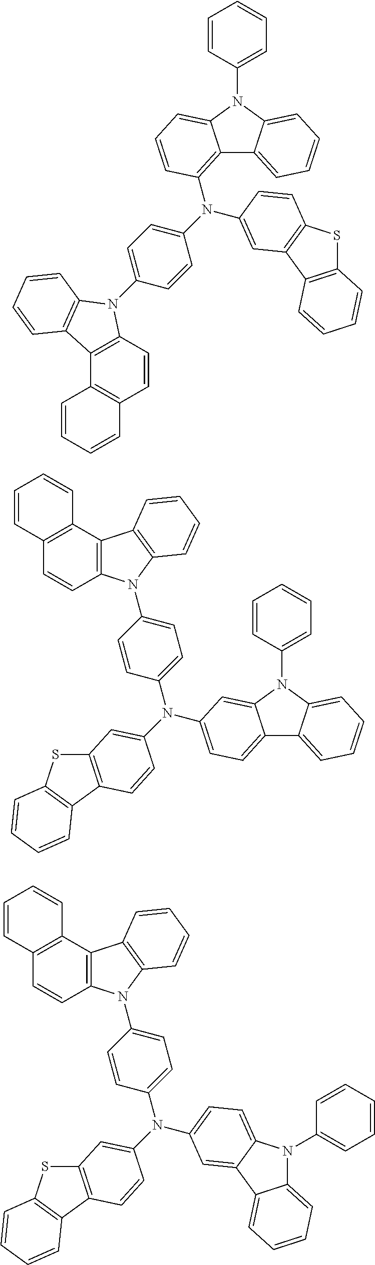 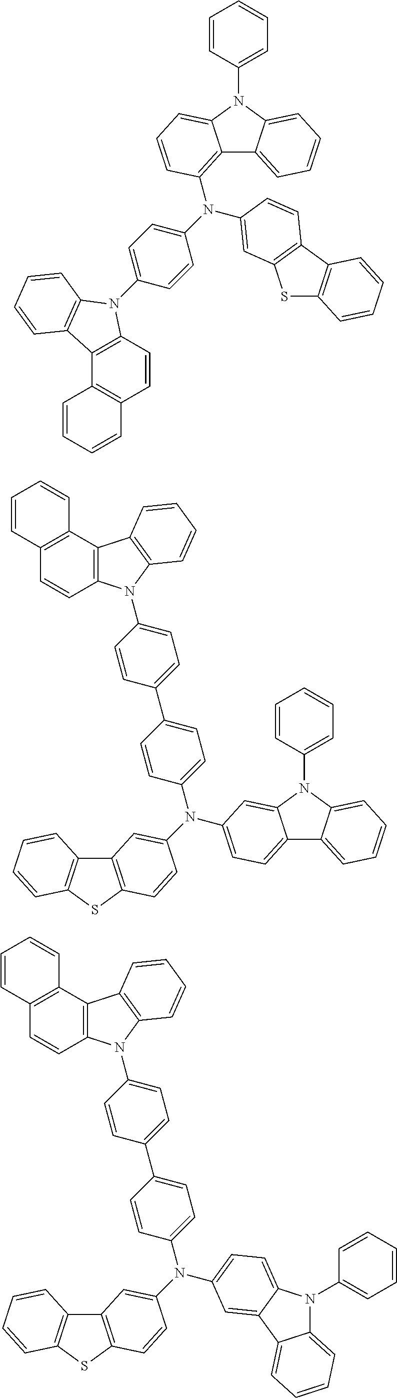 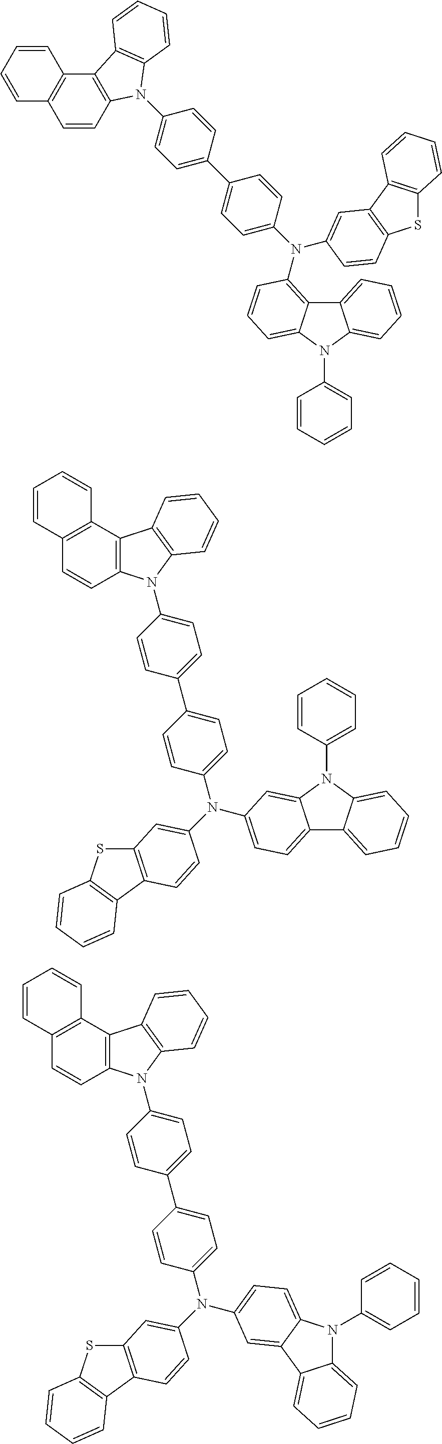 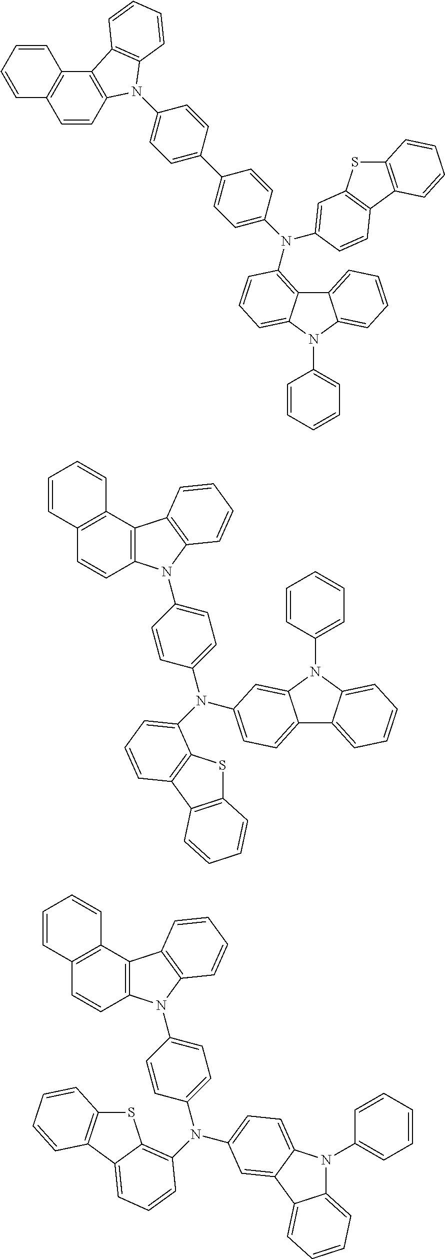 [SMILES: c1ccc(-n2c3ccccc3c3c(N(c4ccc(-c5ccc(-n6c7ccccc7c7c8ccccc8ccc76)cc5)cc4)c4ccc5c(c4)sc4ccccc45)cccc32)cc1 ; c1ccc(-n2c3ccccc3c3ccc(N(c4ccc(-n5c6ccccc6c6c7ccccc7ccc65)cc4)c4cccc5c4sc4ccccc45)cc32)cc1 ; c1ccc(-n2c3ccccc3c3cc(N(c4ccc(-n5c6ccccc6c6c7ccccc7ccc65)cc4)c4cccc5c4sc4ccccc45)ccc32)cc1] 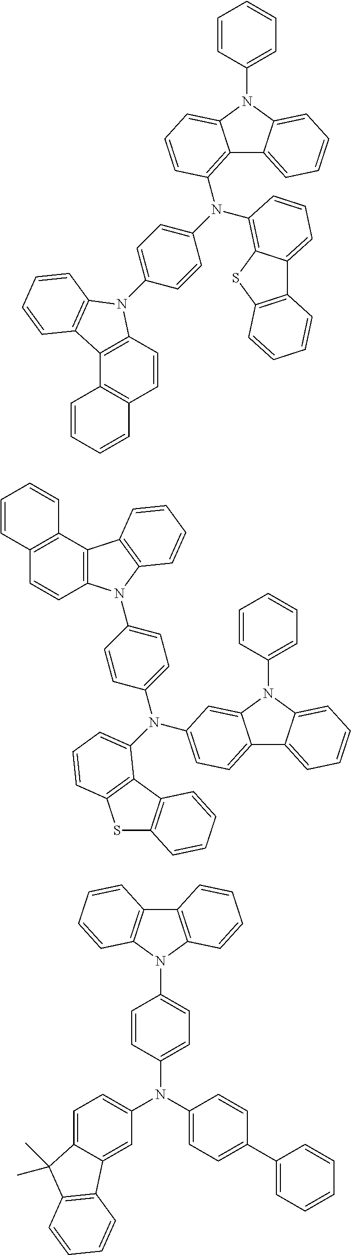 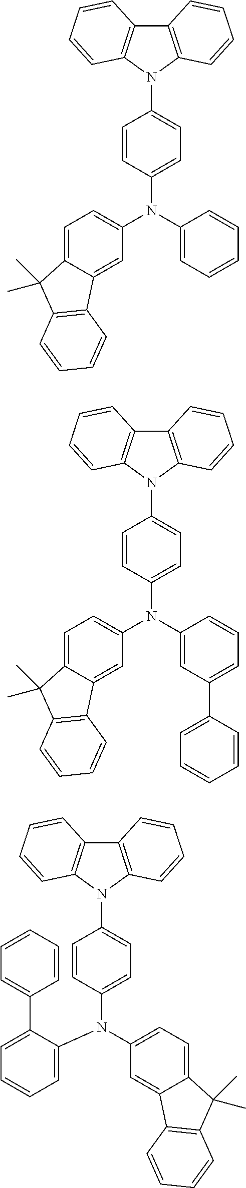 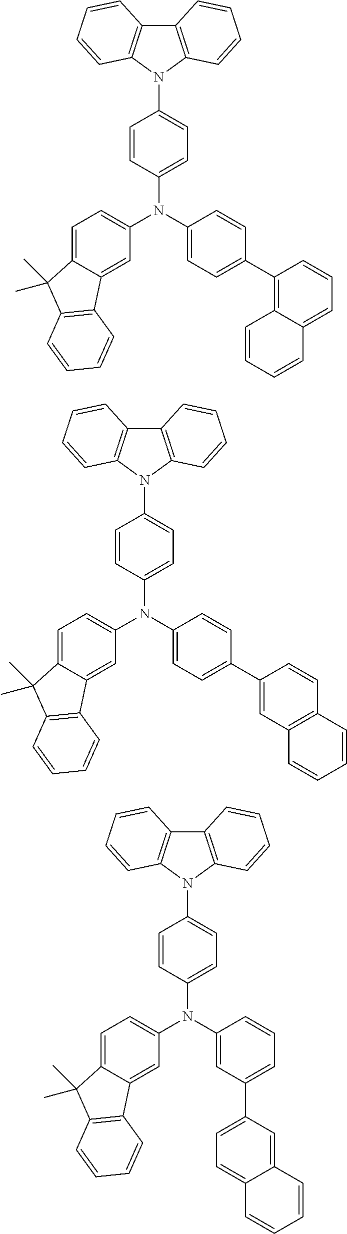 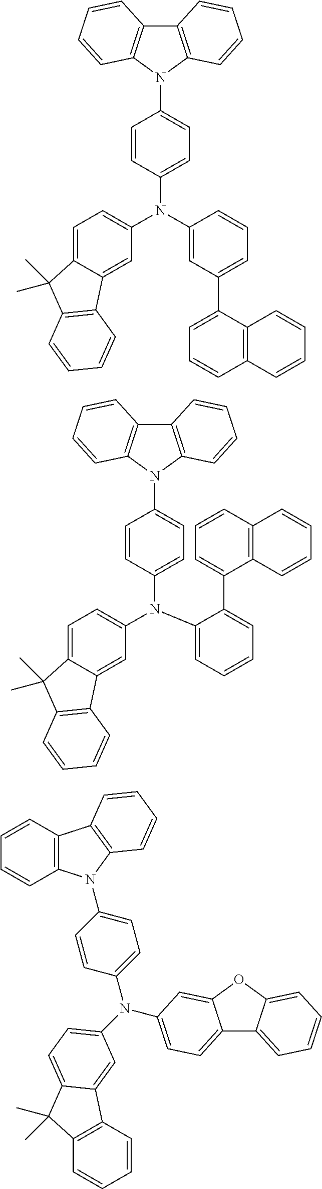 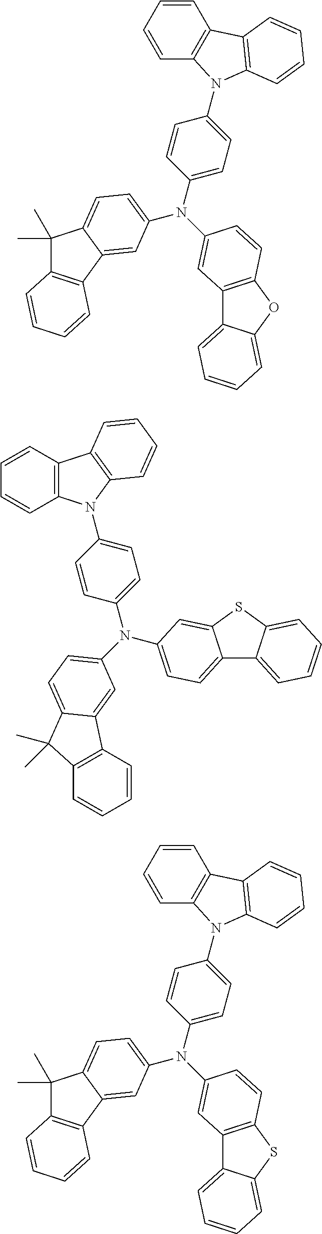 [SMILES: CC1(C)c2ccccc2-c2cc(N(c3ccc(-n4c5ccccc5c5ccccc54)cc3)c3ccc4oc5ccccc5c4c3)ccc21 ; CC1(C)c2ccccc2-c2cc(N(c3ccc(-n4c5ccccc5c5ccccc54)cc3)c3ccc4c(c3)sc3ccccc34)ccc21 ; CC1(C)c2ccccc2-c2cc(N(c3ccc(-n4c5ccccc5c5ccccc54)cc3)c3ccc4sc5ccccc5c4c3)ccc21] 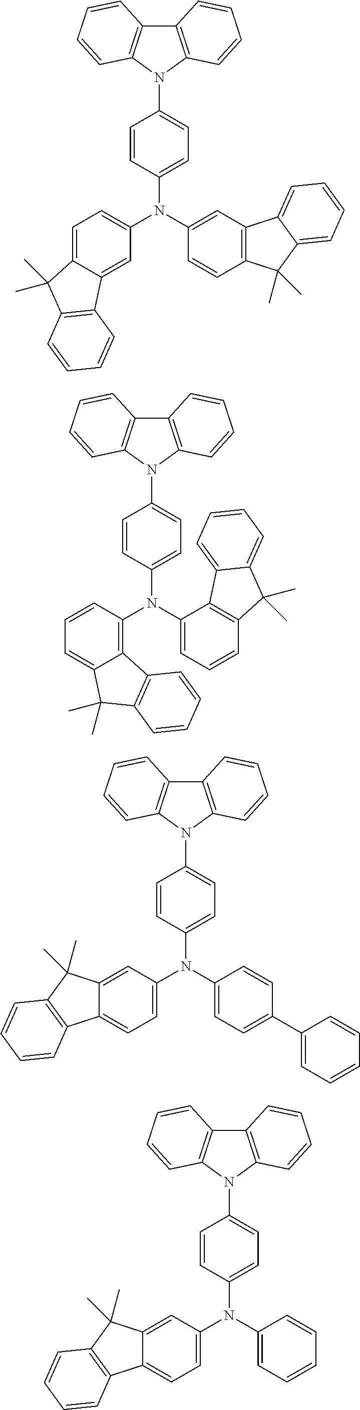 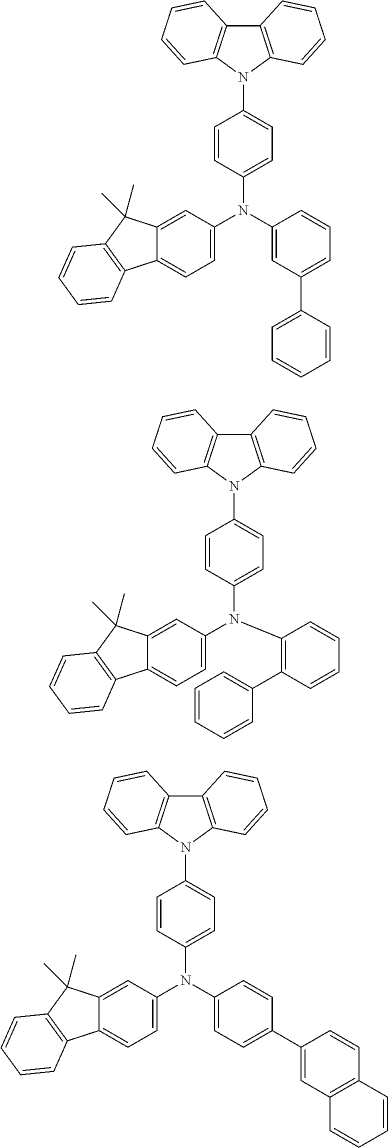 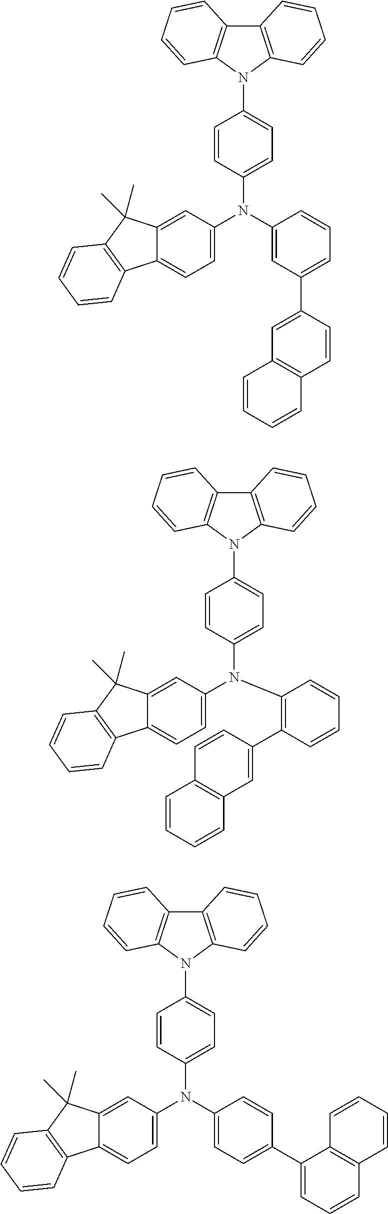 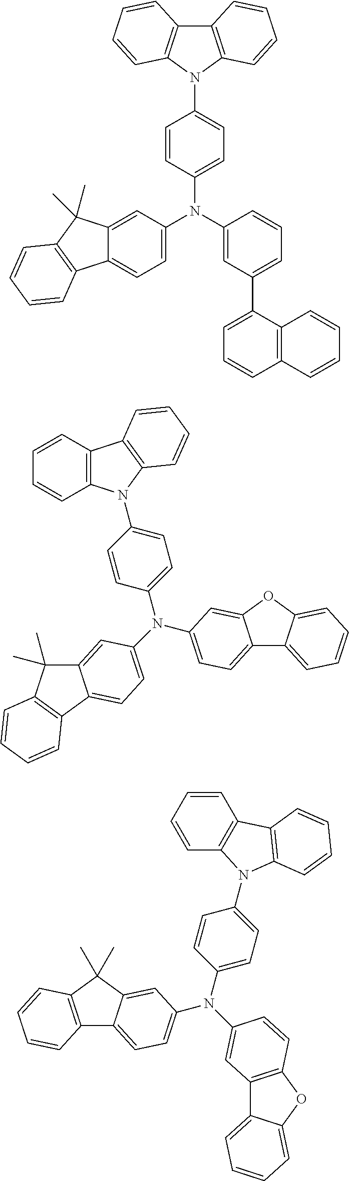 [SMILES: CC1(C)c2ccccc2-c2ccc(N(c3ccc(-n4c5ccccc5c5ccccc54)cc3)c3cccc(-c4cccc5ccccc45)c3)cc21 ; CC1(C)c2ccccc2-c2ccc(N(c3ccc(-n4c5ccccc5c5ccccc54)cc3)c3ccc4c(c3)oc3ccccc34)cc21 ; CC1(C)c2ccccc2-c2ccc(N(c3ccc(-n4c5ccccc5c5ccccc54)cc3)c3ccc4oc5ccccc5c4c3)cc21] 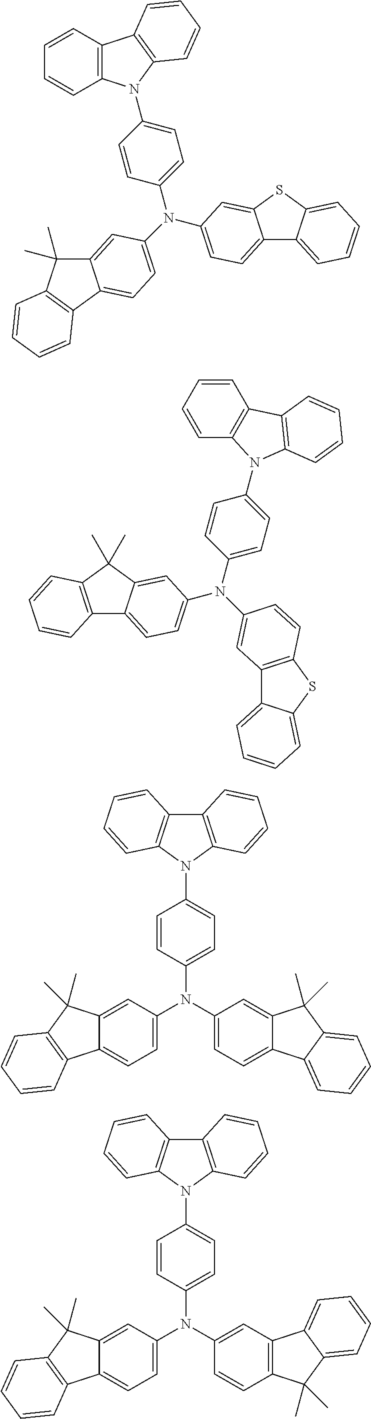 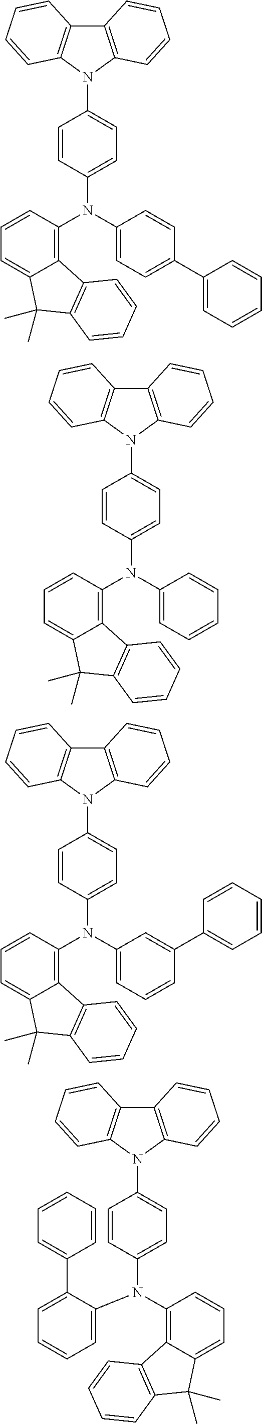 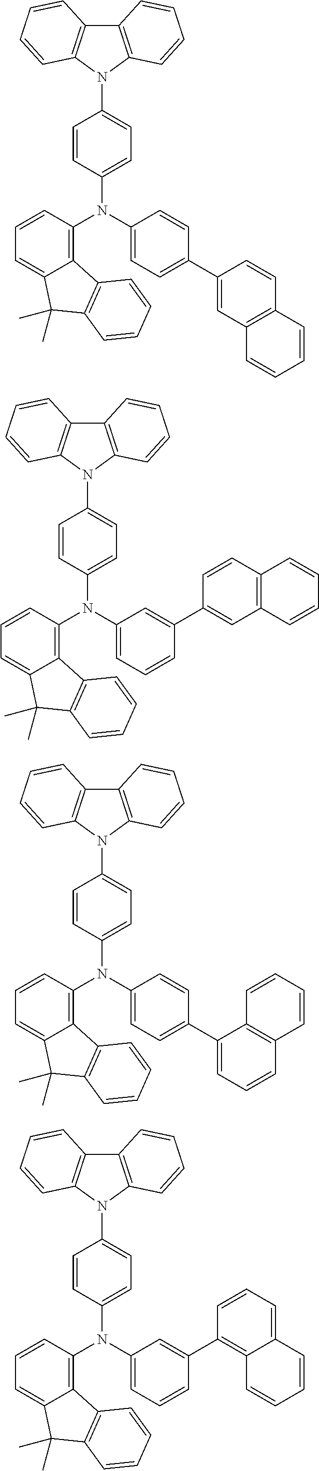 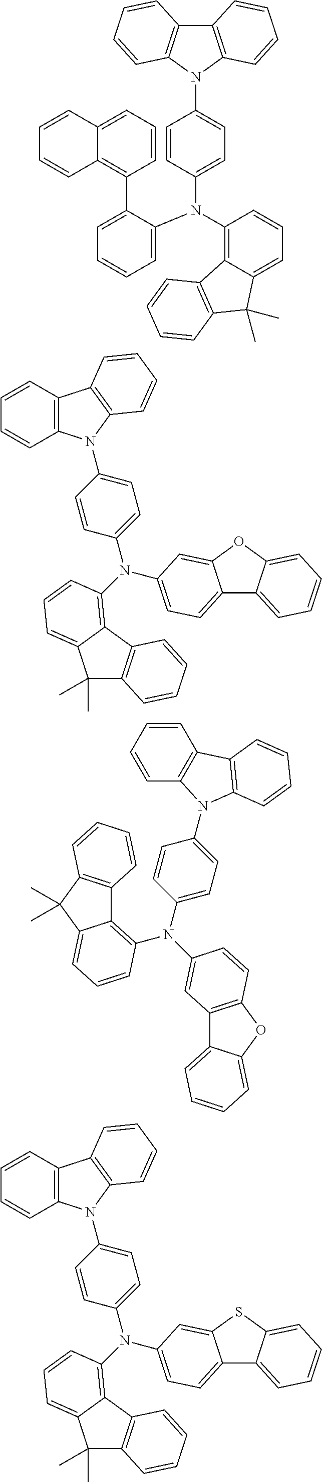 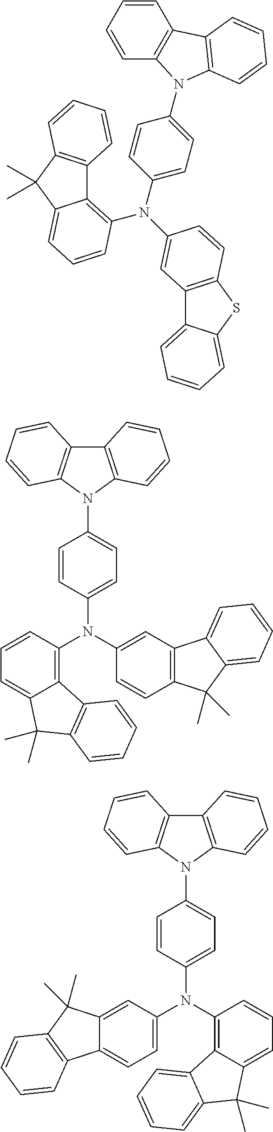 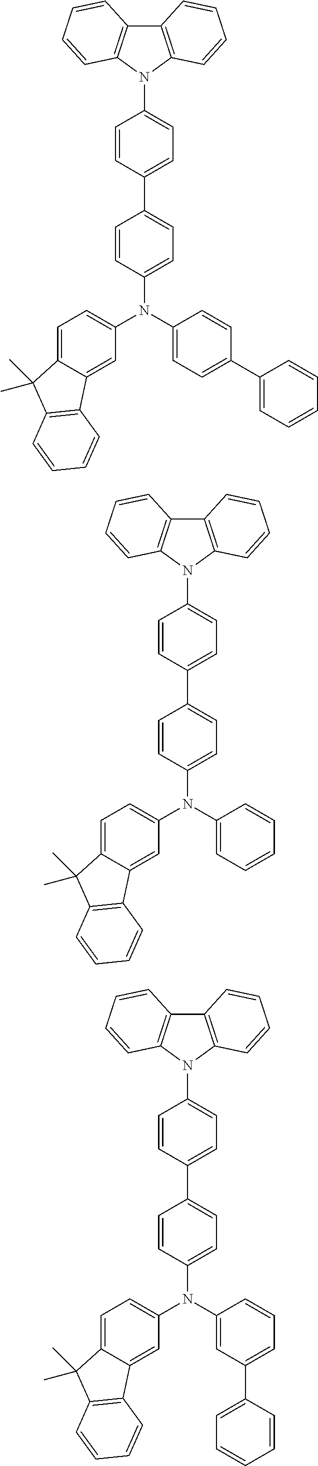 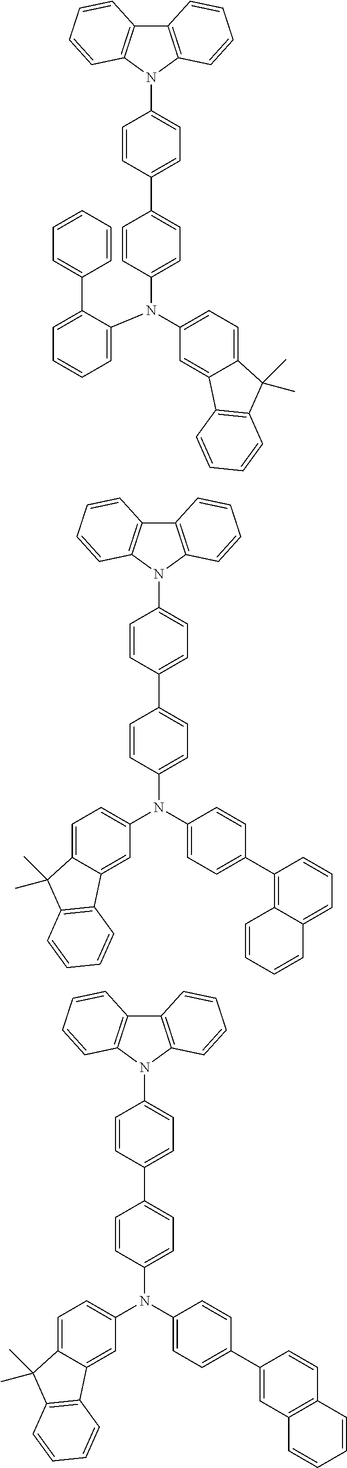 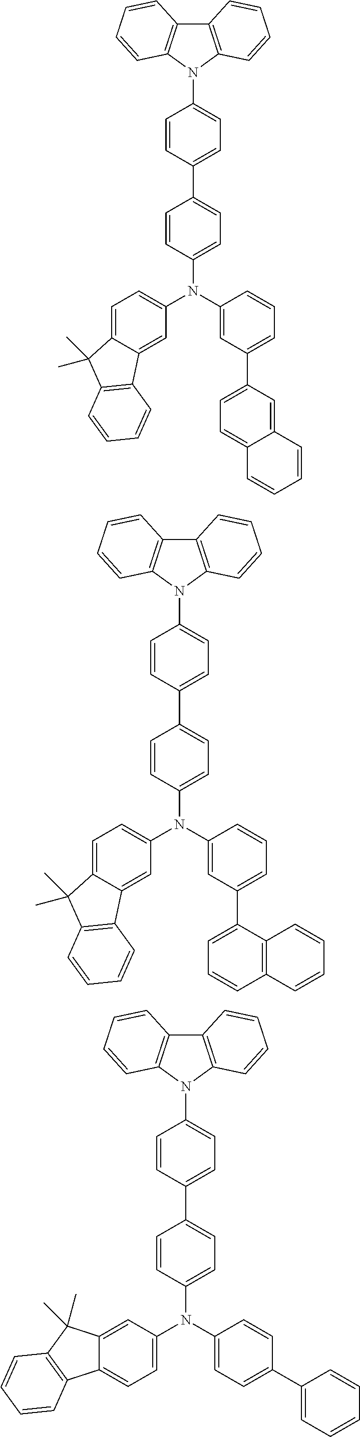 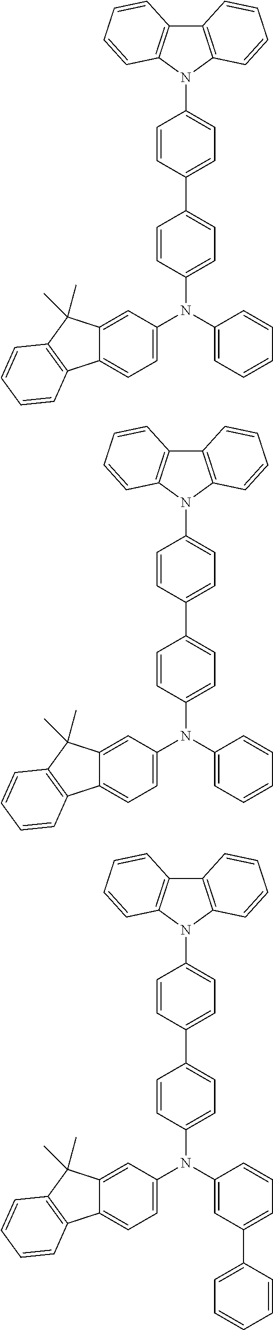 [SMILES: CC1(C)c2ccccc2-c2ccc(N(c3ccccc3)c3ccc(-c4ccc(-n5c6ccccc6c6ccccc65)cc4)cc3)cc21 ; CC1(C)c2ccccc2-c2ccc(N(c3ccccc3)c3ccc(-c4ccc(-n5c6ccccc6c6ccccc65)cc4)cc3)cc21 ; CC1(C)c2ccccc2-c2ccc(N(c3ccc(-c4ccc(-n5c6ccccc6c6ccccc65)cc4)cc3)c3cccc(-c4ccccc4)c3)cc21] 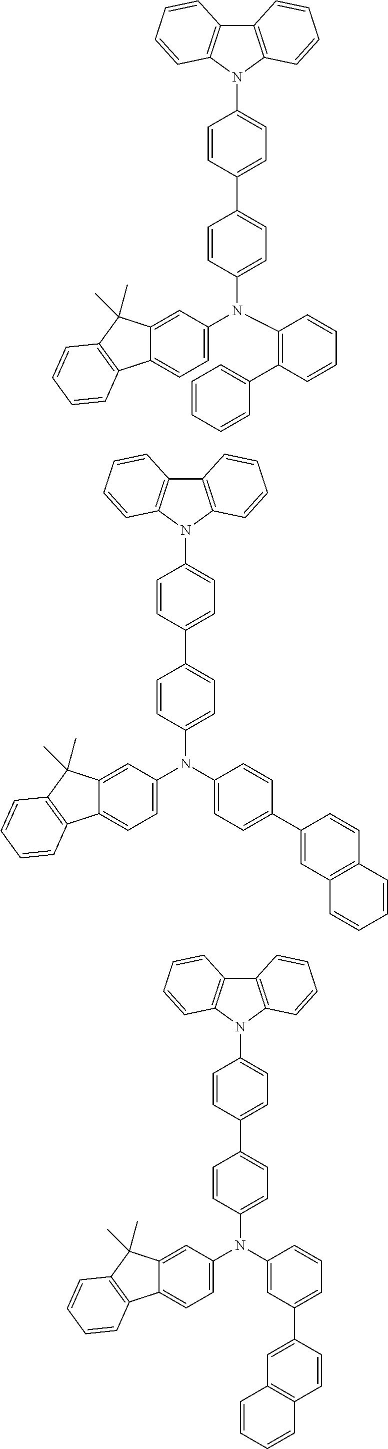 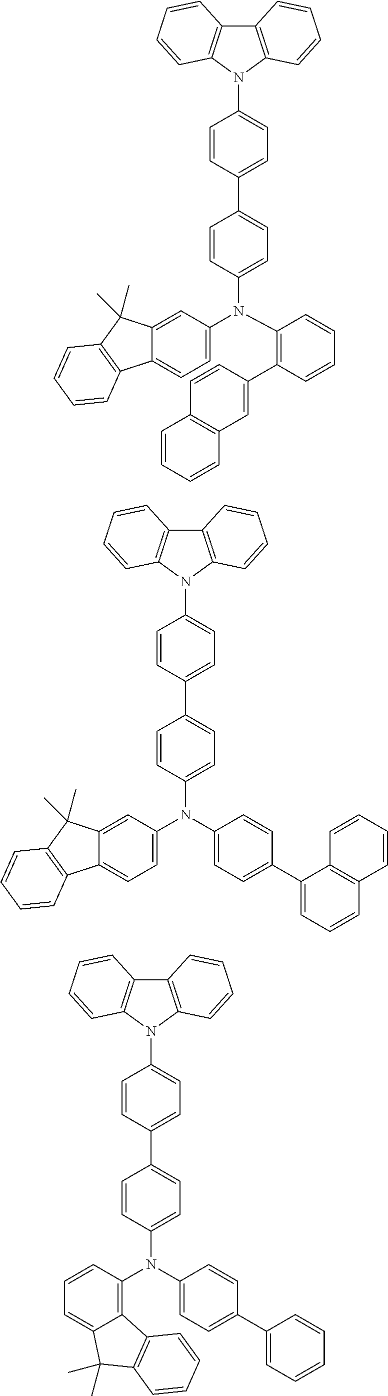 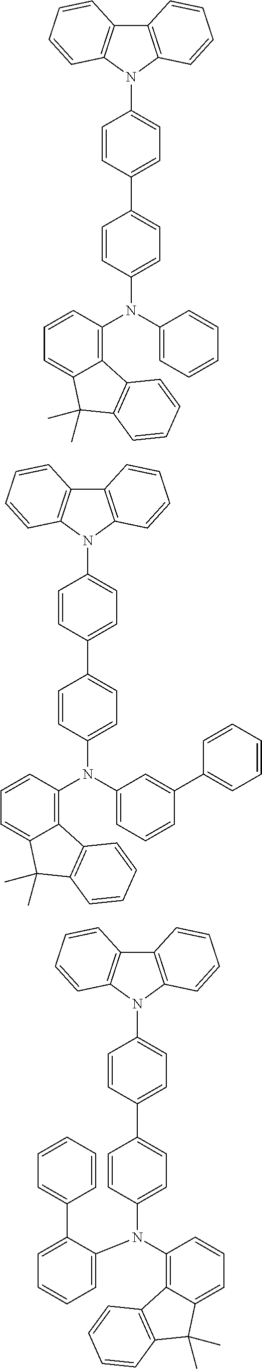 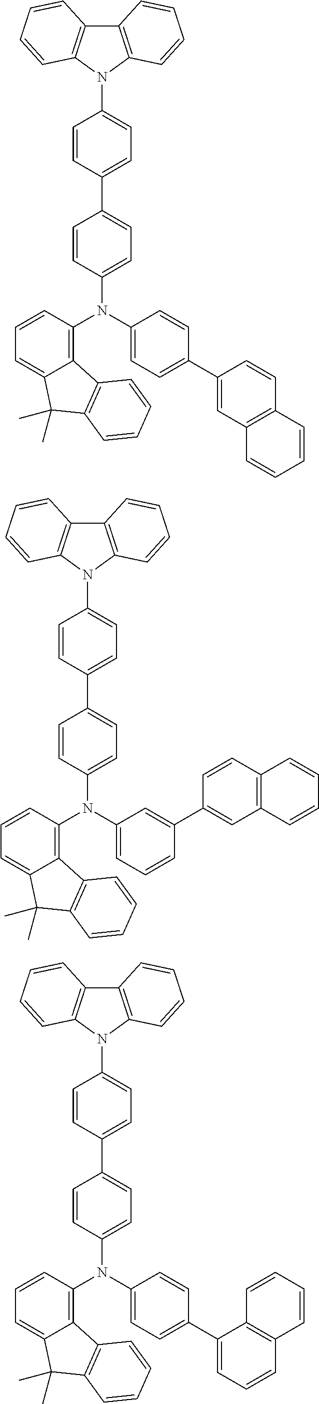 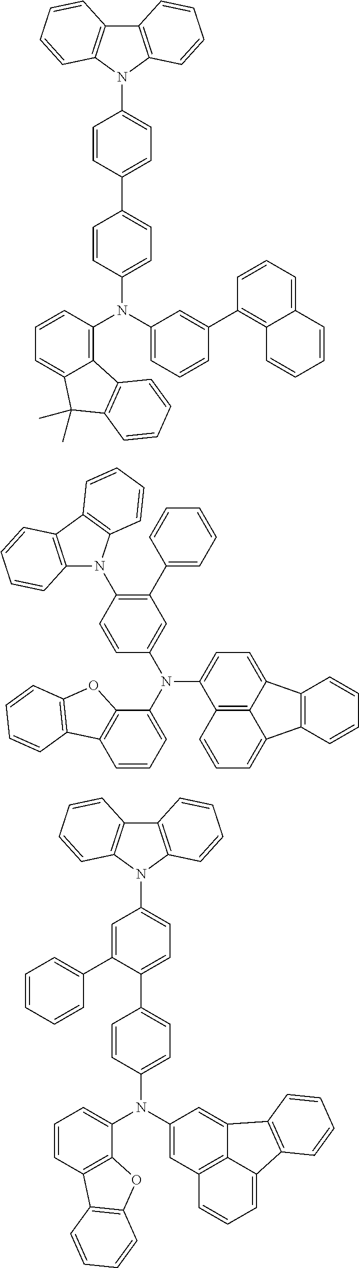 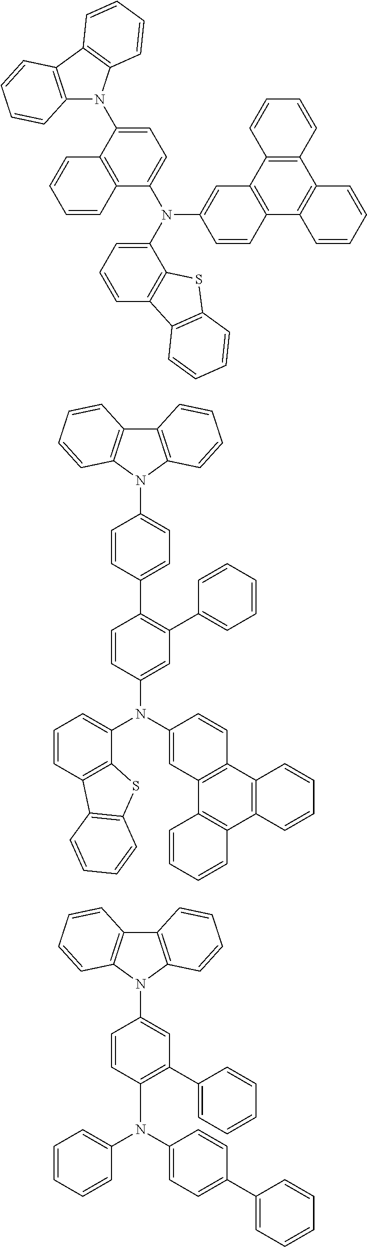 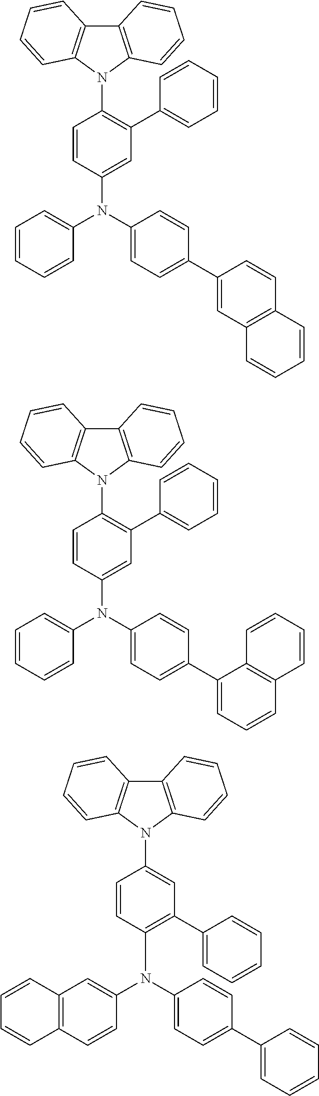 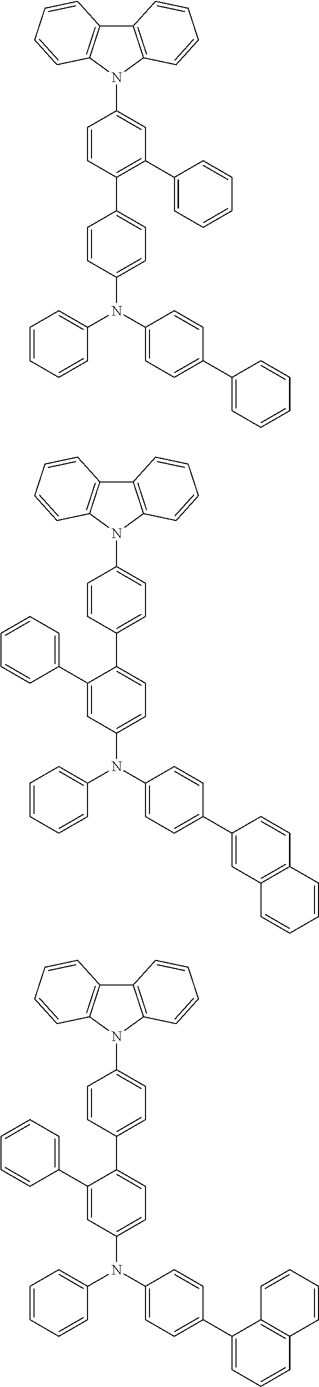 [SMILES: c1ccc(-c2ccc(N(c3ccccc3)c3ccc(-c4ccc(-n5c6ccccc6c6ccccc65)cc4-c4ccccc4)cc3)cc2)cc1 ; c1ccc(-c2cc(N(c3ccccc3)c3ccc(-c4ccc5ccccc5c4)cc3)ccc2-c2ccc(-n3c4ccccc4c4ccccc43)cc2)cc1 ; c1ccc(-c2cc(N(c3ccccc3)c3ccc(-c4cccc5ccccc45)cc3)ccc2-c2ccc(-n3c4ccccc4c4ccccc43)cc2)cc1] 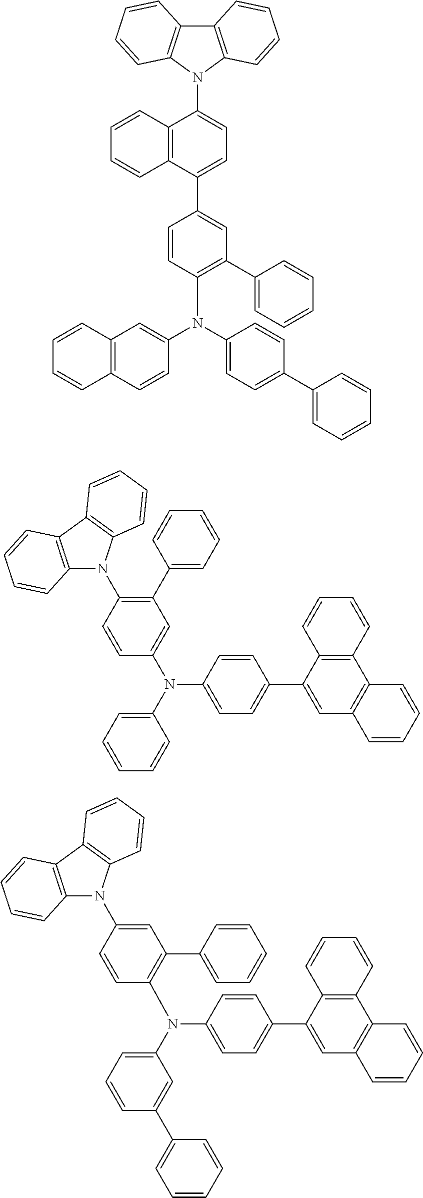 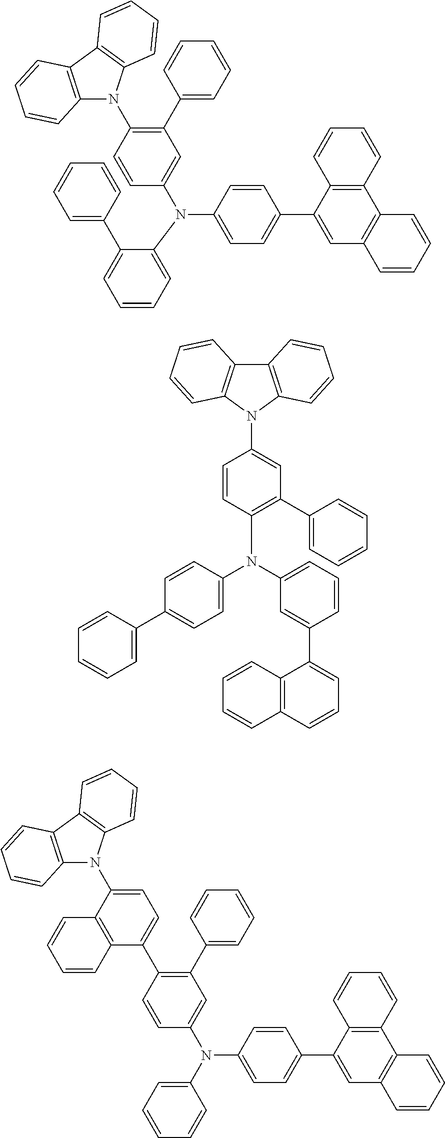 [SMILES: c1ccc(-c2ccccc2N(c2ccc(-c3cc4ccccc4c4ccccc34)cc2)c2ccc(-n3c4ccccc4c4ccccc43)c(-c3ccccc3)c2)cc1 ; c1ccc(-c2ccc(N(c3cccc(-c4cccc5ccccc45)c3)c3ccc(-n4c5ccccc5c5ccccc54)cc3-c3ccccc3)cc2)cc1 ; c1ccc(-c2cc(N(c3ccccc3)c3ccc(-c4cc5ccccc5c5ccccc45)cc3)ccc2-c2ccc(-n3c4ccccc4c4ccccc43)c3ccccc23)cc1] 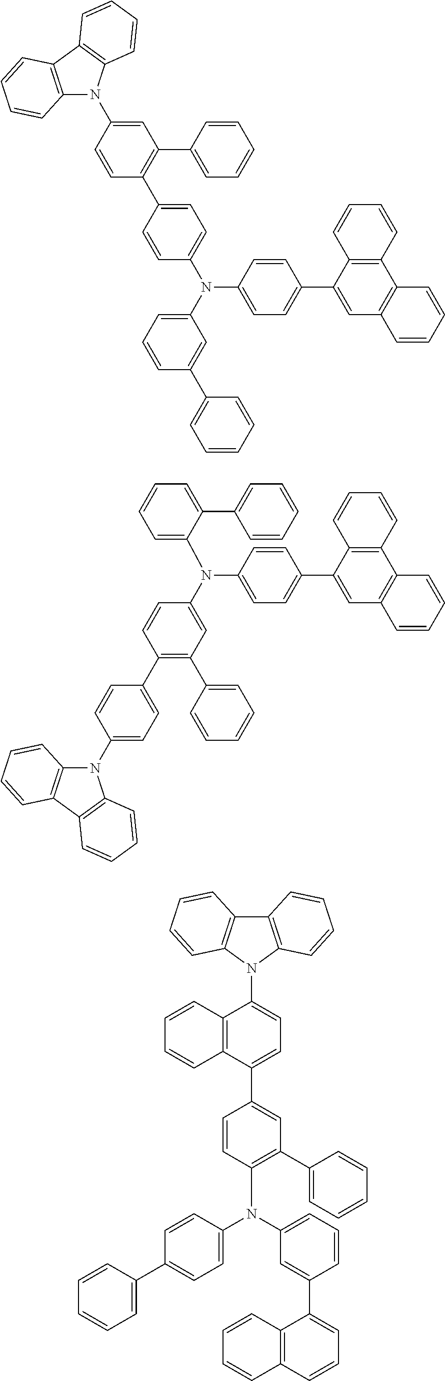 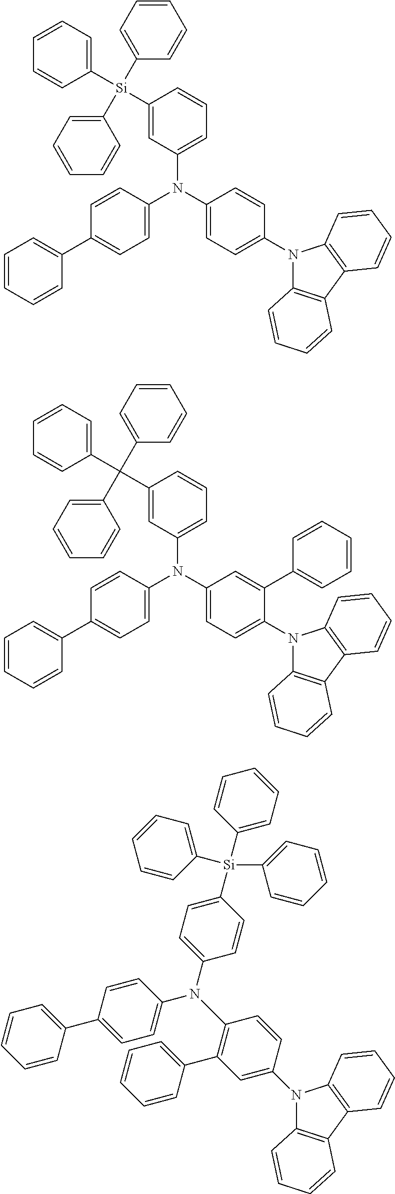 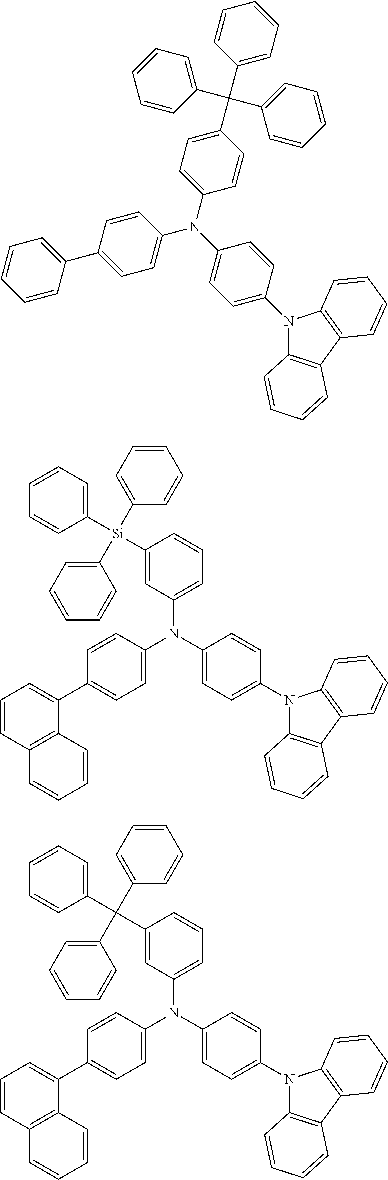 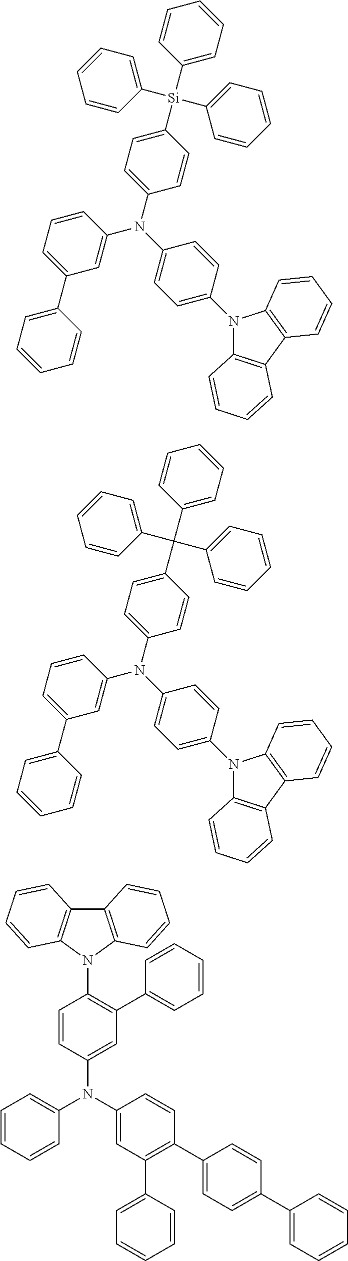 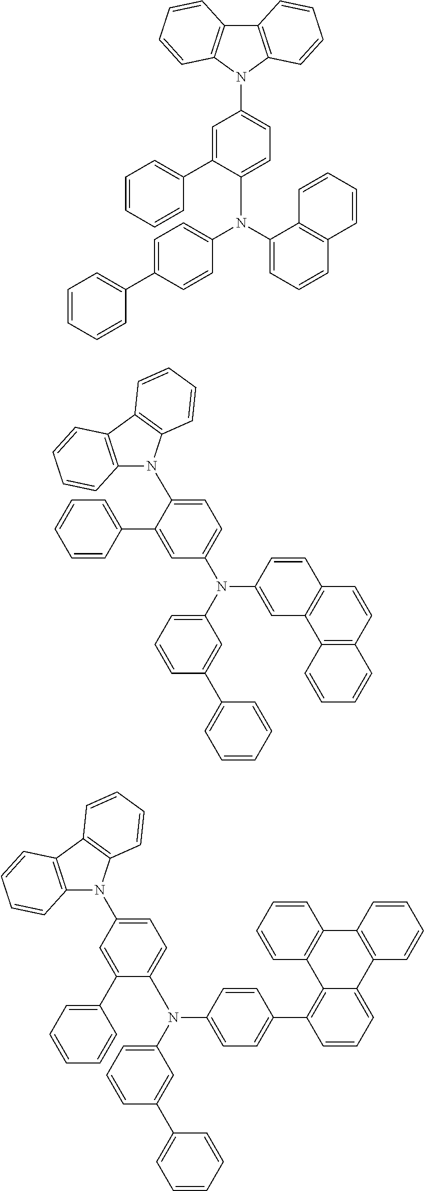 [SMILES: c1ccc(-c2ccc(N(c3ccc(-n4c5ccccc5c5ccccc54)cc3-c3ccccc3)c3cccc4ccccc34)cc2)cc1 ; c1ccc(-c2cccc(N(c3ccc(-n4c5ccccc5c5ccccc54)c(-c4ccccc4)c3)c3ccc4ccc5ccccc5c4c3)c2)cc1 ; c1ccc(-c2cccc(N(c3ccc(-c4cccc5c6ccccc6c6ccccc6c45)cc3)c3ccc(-n4c5ccccc5c5ccccc54)cc3-c3ccccc3)c2)cc1] 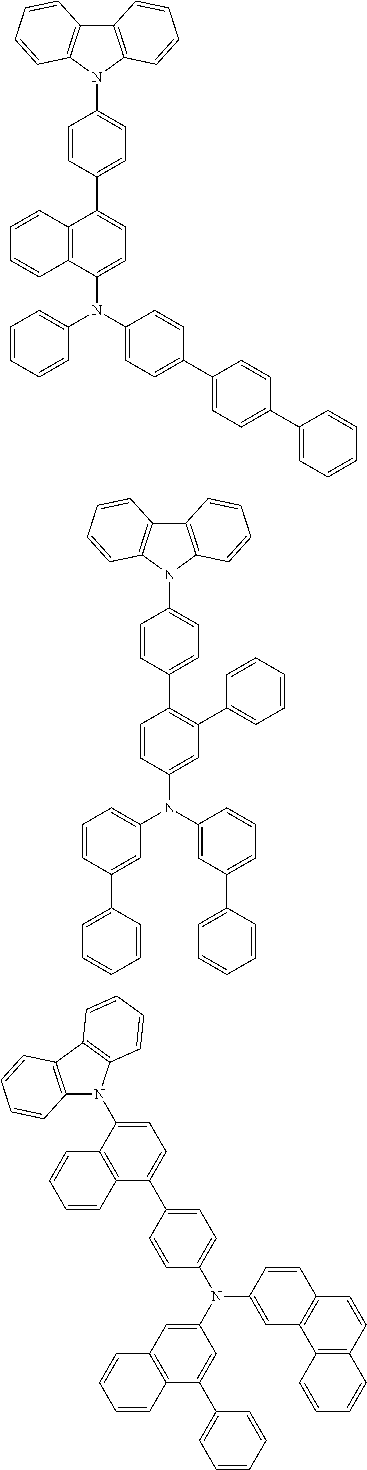 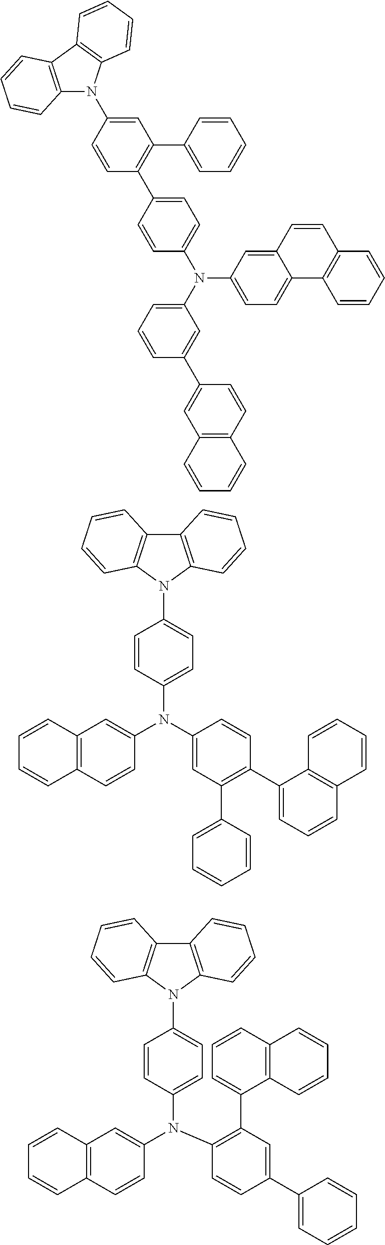 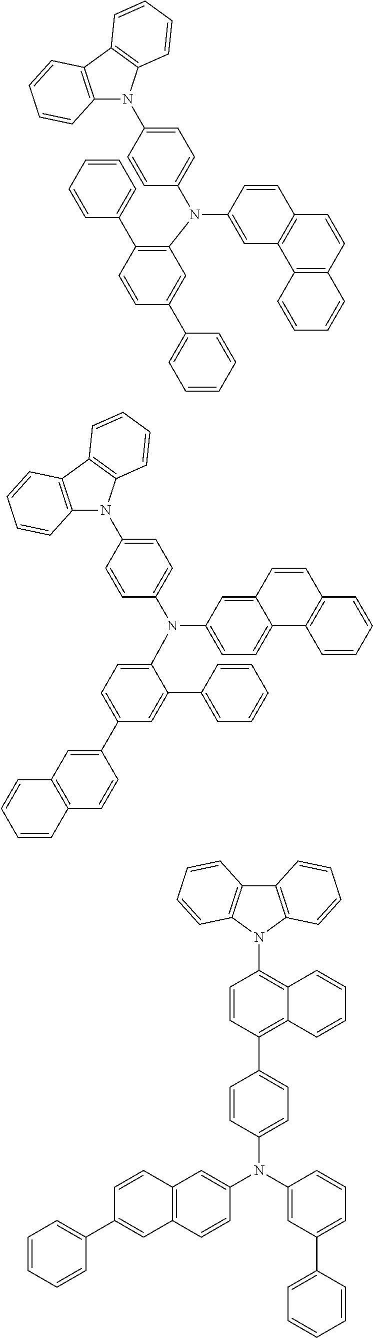 [SMILES: c1ccc(-c2ccc(-c3ccccc3)c(N(c3ccc(-n4c5ccccc5c5ccccc54)cc3)c3ccc4ccc5ccccc5c4c3)c2)cc1 ; c1ccc(-c2cc(-c3ccc4ccccc4c3)ccc2N(c2ccc(-n3c4ccccc4c4ccccc43)cc2)c2ccc3c(ccc4ccccc43)c2)cc1 ; c1ccc(-c2cccc(N(c3ccc(-c4ccc(-n5c6ccccc6c6ccccc65)c5ccccc45)cc3)c3ccc4cc(-c5ccccc5)ccc4c3)c2)cc1] 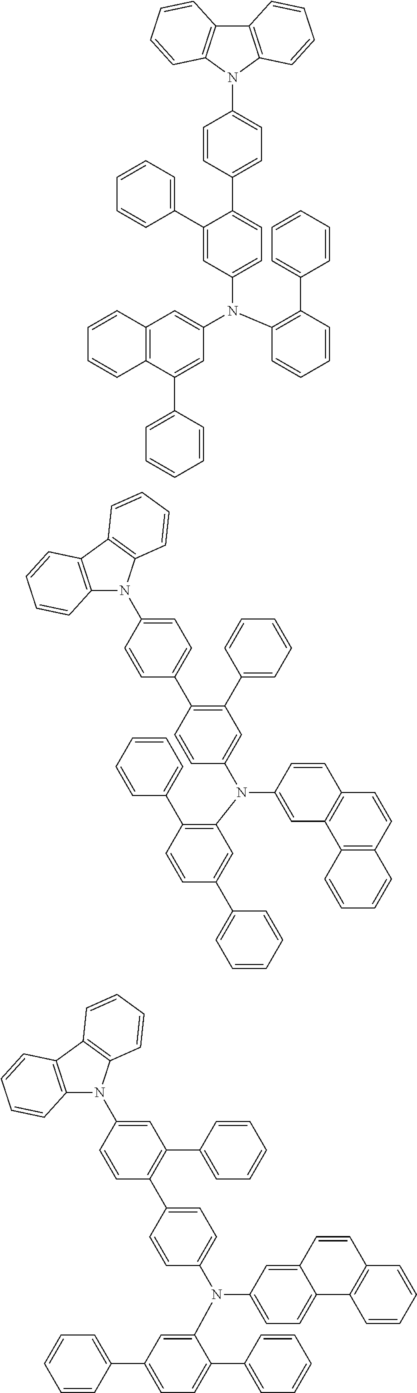 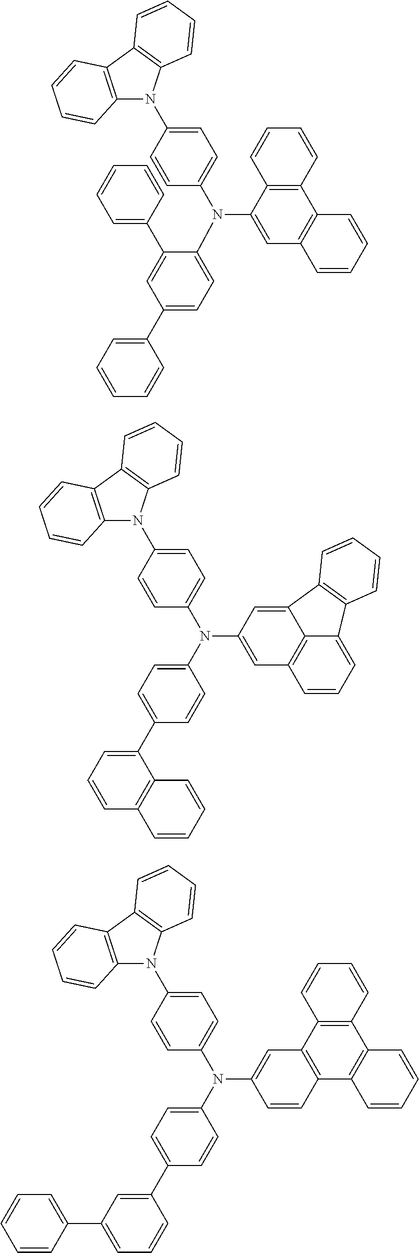 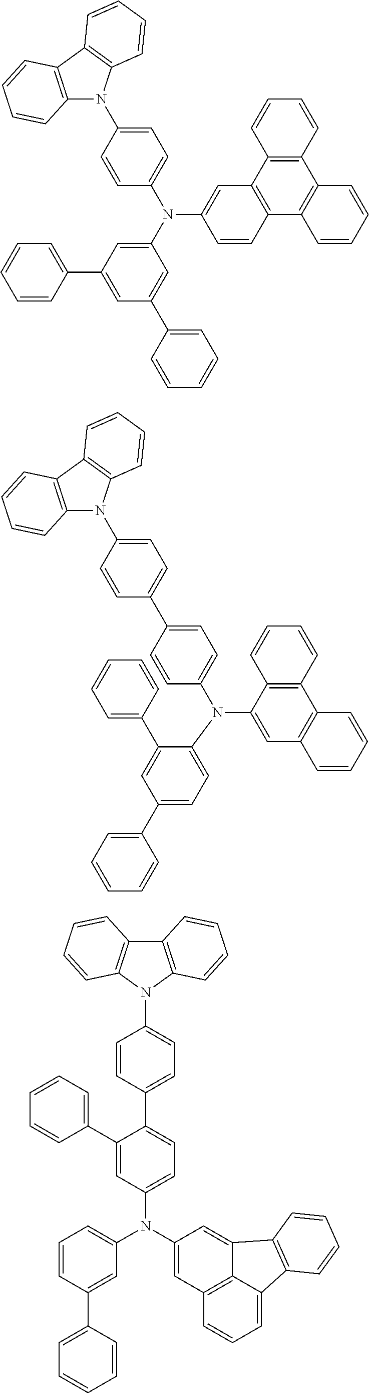 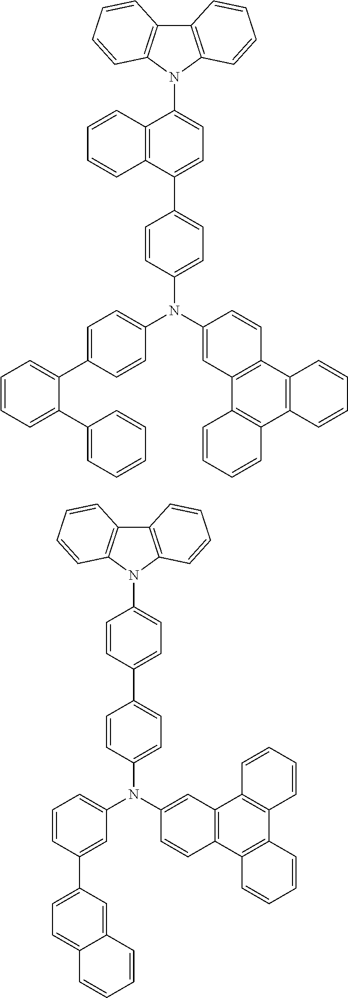 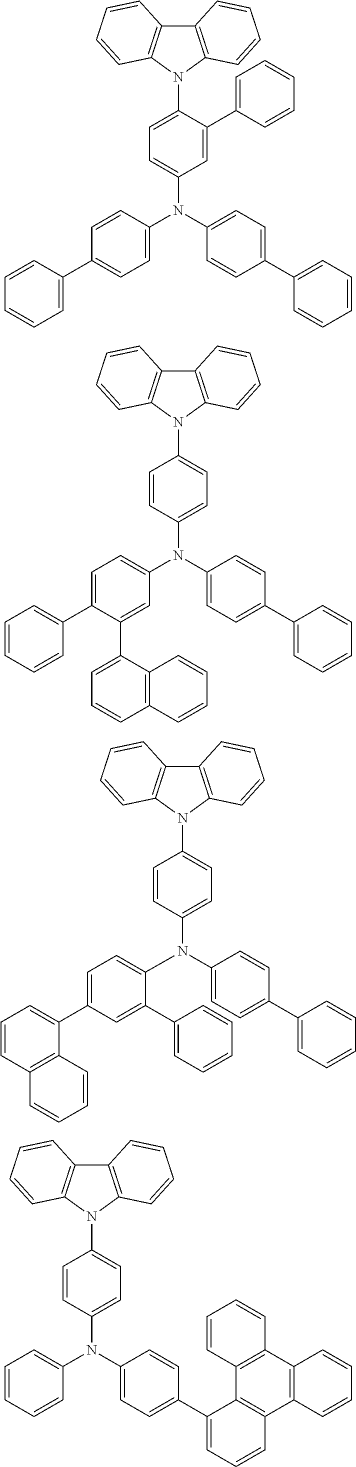 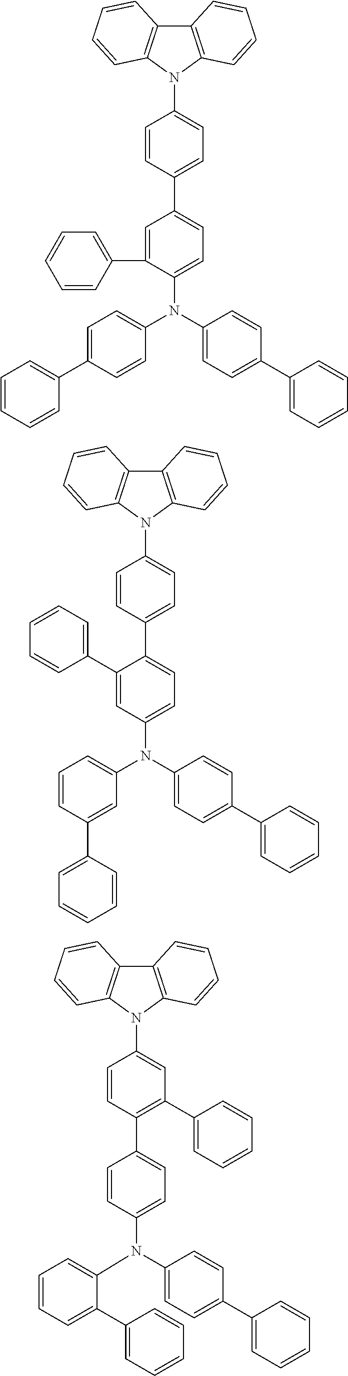 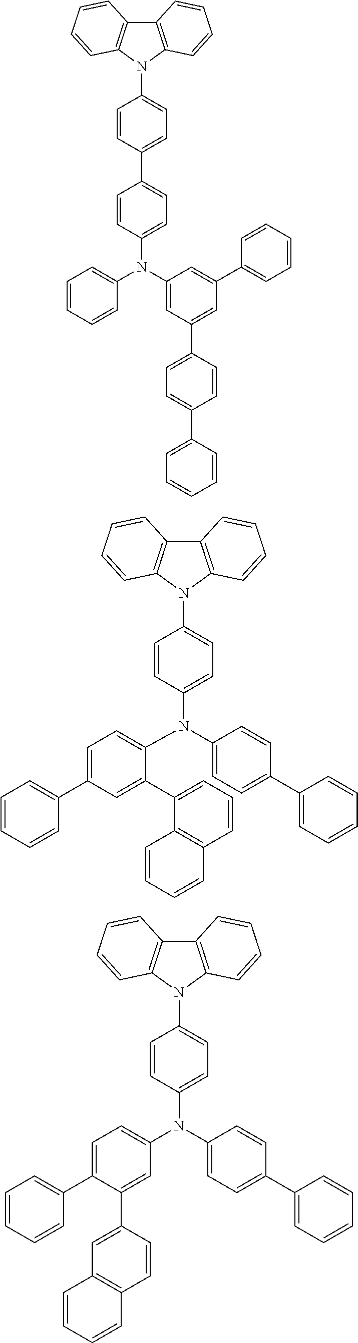 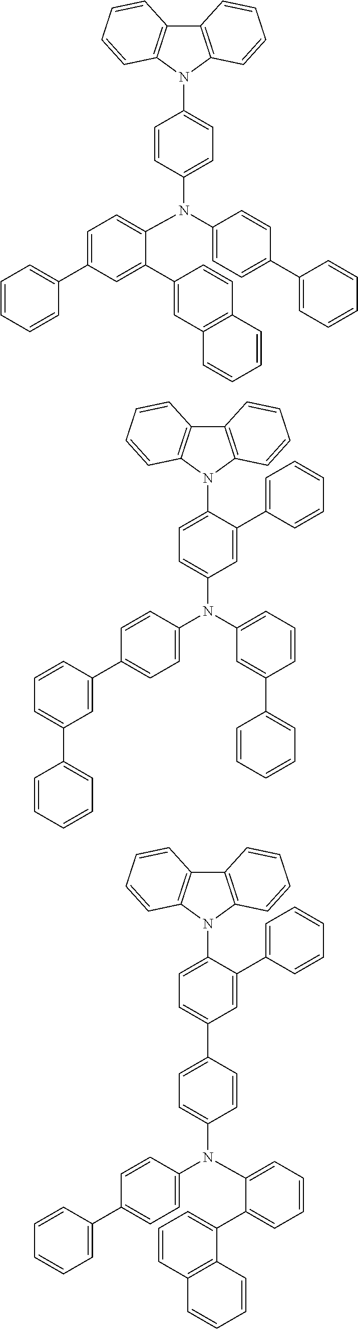 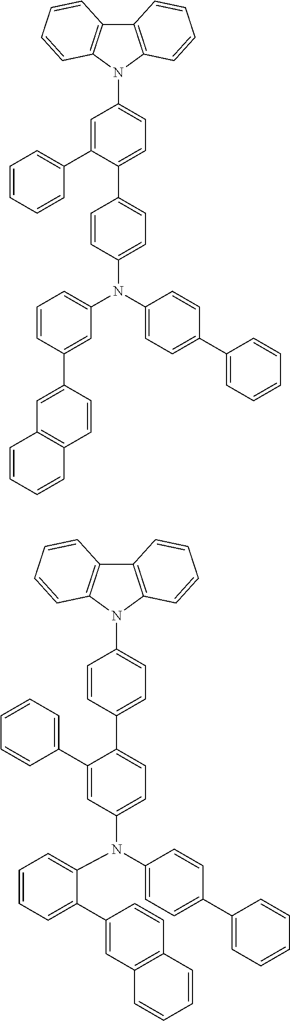 [SMILES: c1ccc(-c2ccc(N(c3ccc(-c4ccc(-n5c6ccccc6c6ccccc65)cc4-c4ccccc4)cc3)c3cccc(-c4ccc5ccccc5c4)c3)cc2)cc1 ; c1ccc(-c2ccc(N(c3ccc(-c4ccc(-n5c6ccccc6c6ccccc65)cc4)c(-c4ccccc4)c3)c3ccccc3-c3ccc4ccccc4c3)cc2)cc1] 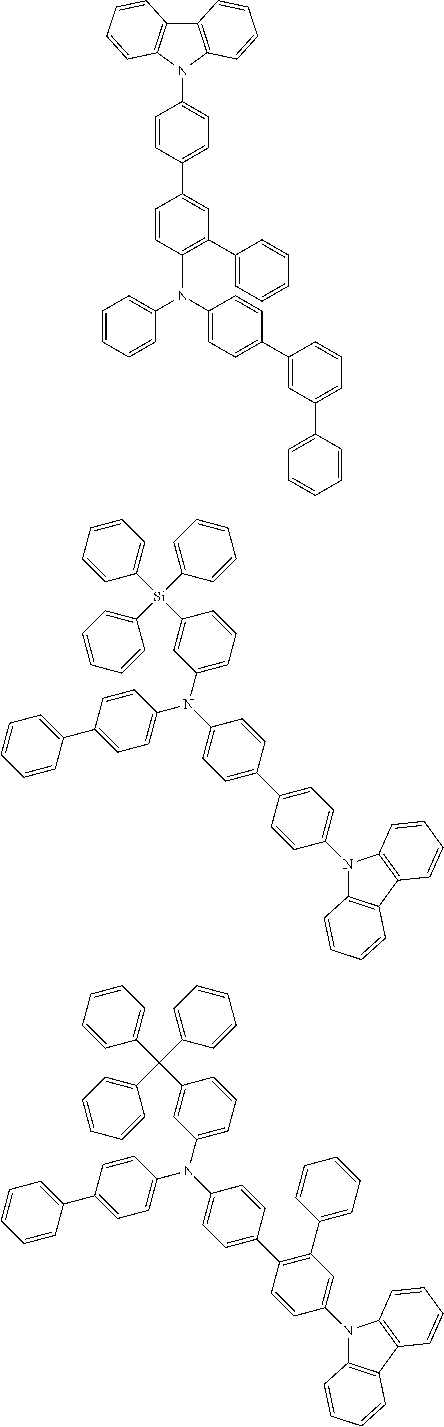 [SMILES: c1ccc(-c2cccc(-c3ccc(N(c4ccccc4)c4ccc(-c5ccc(-n6c7ccccc7c7ccccc76)cc5)cc4-c4ccccc4)cc3)c2)cc1 ; c1ccc(-c2ccc(N(c3ccc(-c4ccc(-n5c6ccccc6c6ccccc65)cc4)cc3)c3cccc([Si](c4ccccc4)(c4ccccc4)c4ccccc4)c3)cc2)cc1 ; c1ccc(-c2ccc(N(c3ccc(-c4ccc(-n5c6ccccc6c6ccccc65)cc4-c4ccccc4)cc3)c3cccc(C(c4ccccc4)(c4ccccc4)c4ccccc4)c3)cc2)cc1] 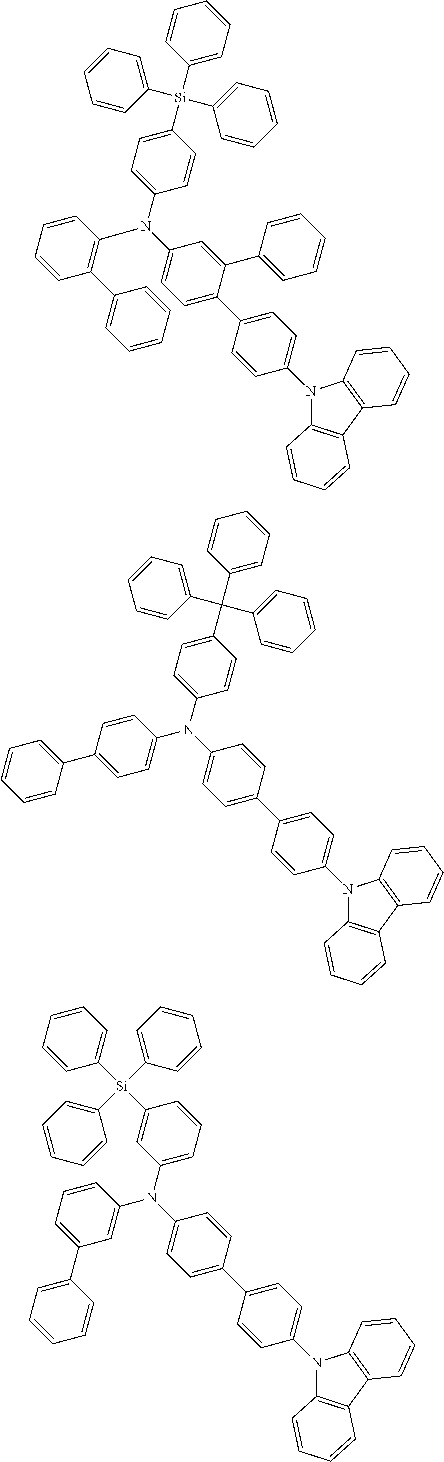 [SMILES: c1ccc(-c2cc(N(c3ccc([Si](c4ccccc4)(c4ccccc4)c4ccccc4)cc3)c3ccccc3-c3ccccc3)ccc2-c2ccc(-n3c4ccccc4c4ccccc43)cc2)cc1 ; c1ccc(-c2ccc(N(c3ccc(-c4ccc(-n5c6ccccc6c6ccccc65)cc4)cc3)c3ccc(C(c4ccccc4)(c4ccccc4)c4ccccc4)cc3)cc2)cc1 ; c1ccc(-c2cccc(N(c3ccc(-c4ccc(-n5c6ccccc6c6ccccc65)cc4)cc3)c3cccc([Si](c4ccccc4)(c4ccccc4)c4ccccc4)c3)c2)cc1] 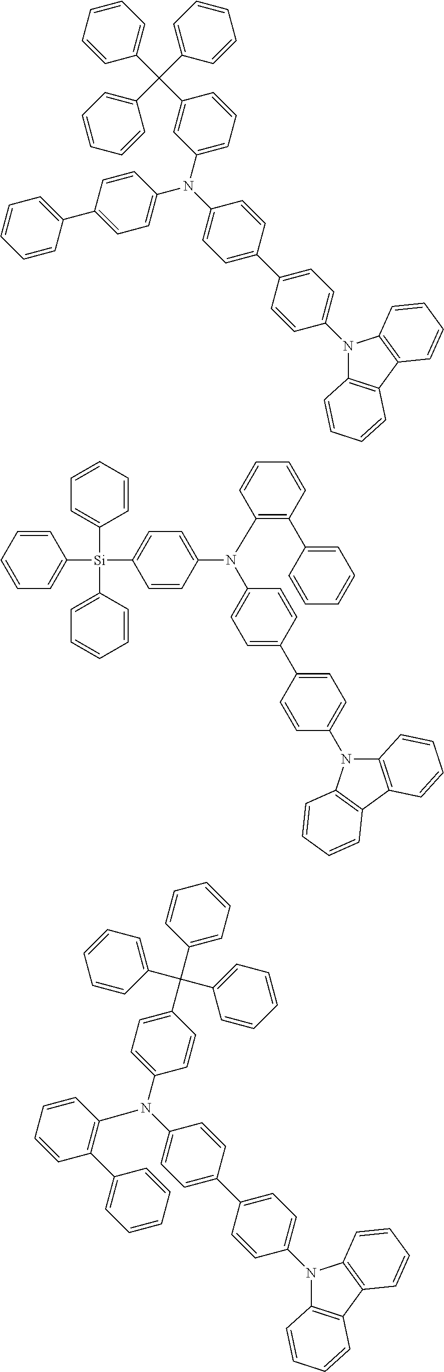 [SMILES: c1ccc(-c2ccc(N(c3ccc(-c4ccc(-n5c6ccccc6c6ccccc65)cc4)cc3)c3cccc(C(c4ccccc4)(c4ccccc4)c4ccccc4)c3)cc2)cc1 ; c1ccc(-c2ccccc2N(c2ccc(-c3ccc(-n4c5ccccc5c5ccccc54)cc3)cc2)c2ccc([Si](c3ccccc3)(c3ccccc3)c3ccccc3)cc2)cc1 ; c1ccc(-c2ccccc2N(c2ccc(-c3ccc(-n4c5ccccc5c5ccccc54)cc3)cc2)c2ccc(C(c3ccccc3)(c3ccccc3)c3ccccc3)cc2)cc1] 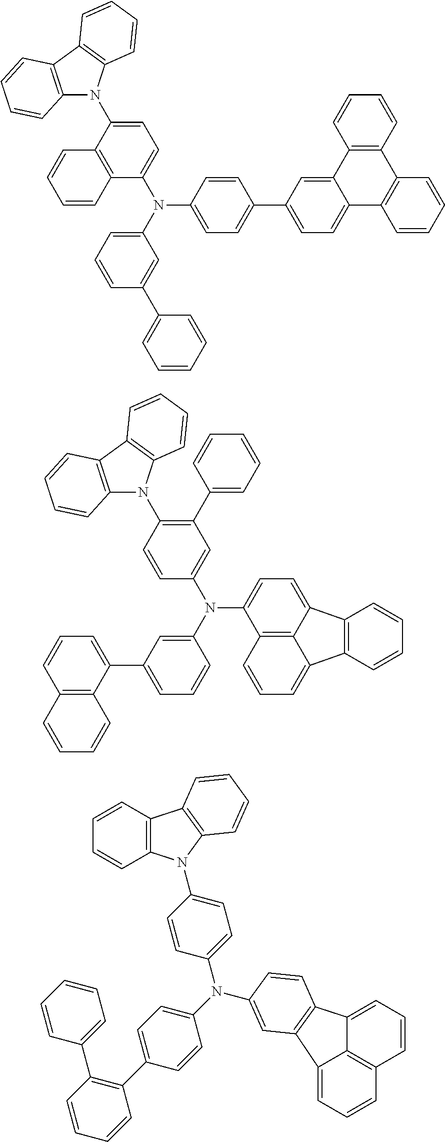 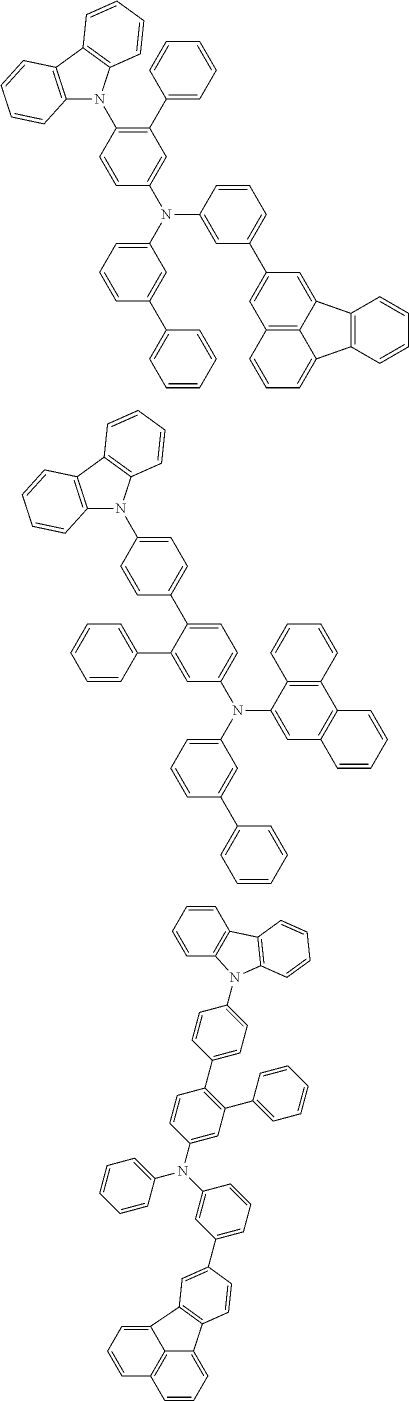 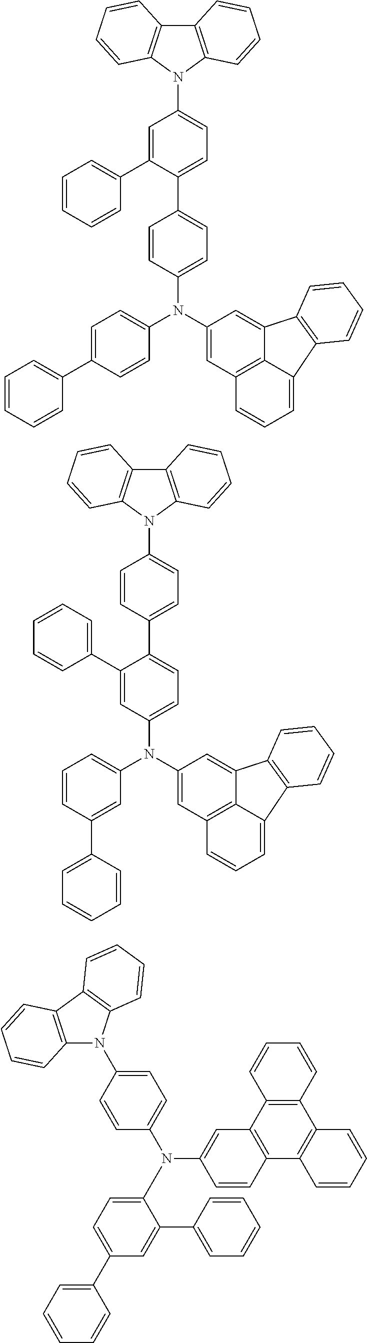 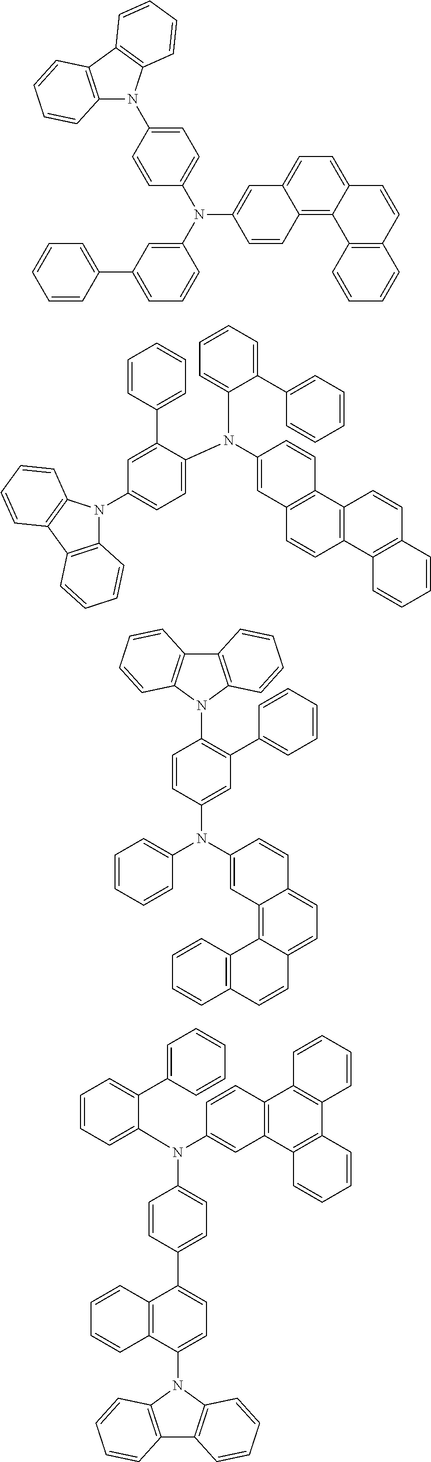 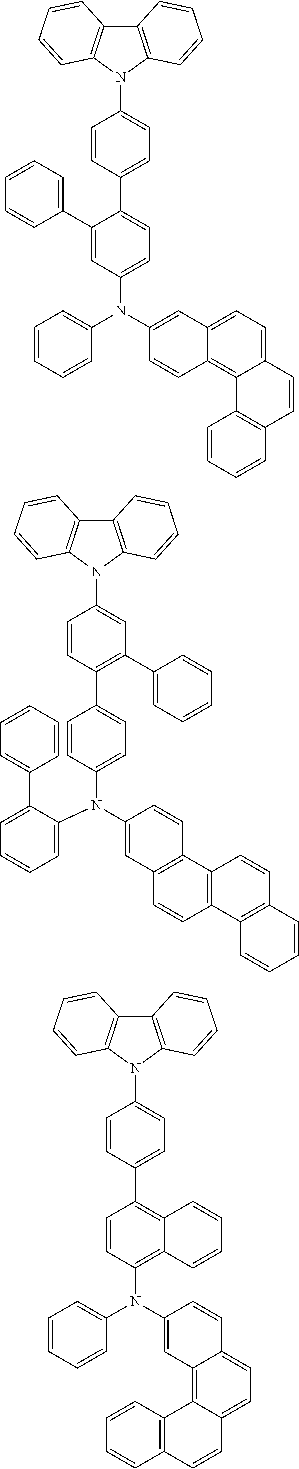 [SMILES: c1ccc(-c2cc(N(c3ccccc3)c3ccc4c(ccc5ccc6ccccc6c54)c3)ccc2-c2ccc(-n3c4ccccc4c4ccccc43)cc2)cc1 ; c1ccc(-c2cc(-n3c4ccccc4c4ccccc43)ccc2-c2ccc(N(c3ccc4c(ccc5c6ccccc6ccc45)c3)c3ccccc3-c3ccccc3)cc2)cc1 ; c1ccc(N(c2ccc3ccc4ccc5ccccc5c4c3c2)c2ccc(-c3ccc(-n4c5ccccc5c5ccccc54)cc3)c3ccccc23)cc1] 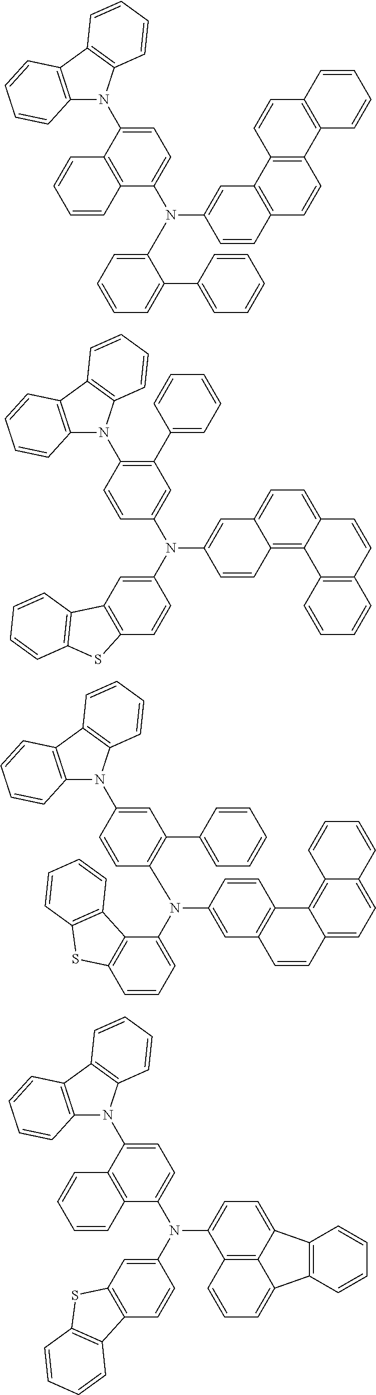 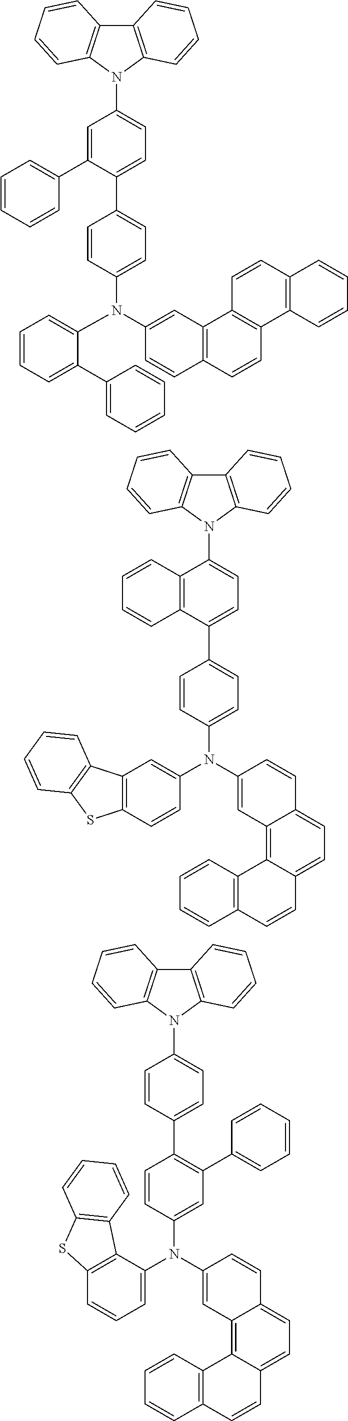 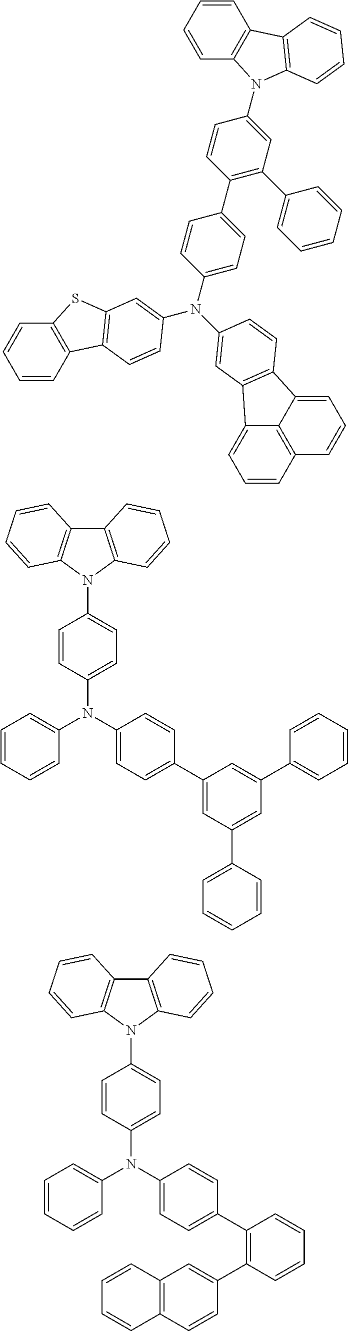 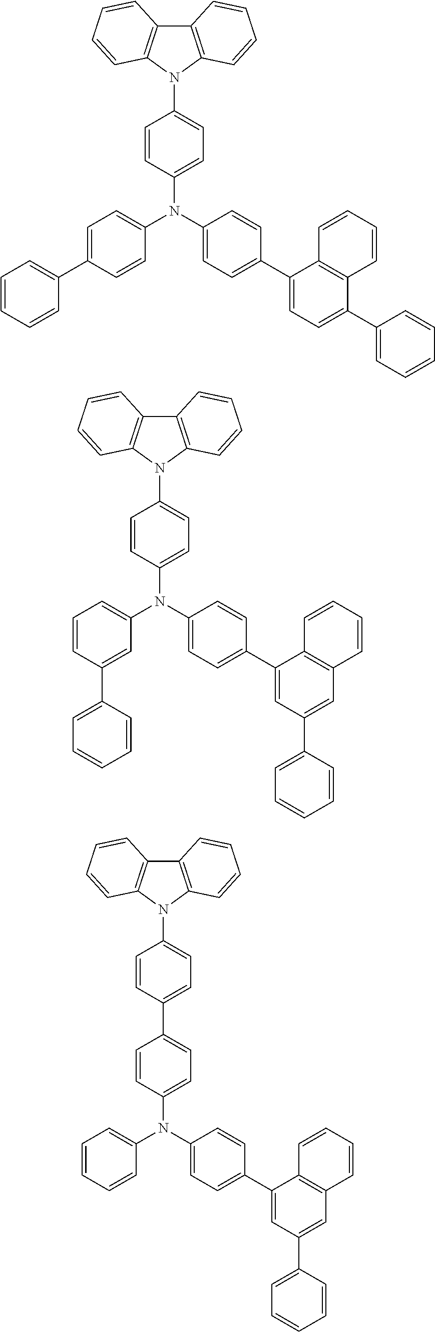 [SMILES: c1ccc(-c2ccc(N(c3ccc(-c4ccc(-c5ccccc5)c5ccccc45)cc3)c3ccc(-n4c5ccccc5c5ccccc54)cc3)cc2)cc1 ; c1ccc(-c2cccc(N(c3ccc(-c4cc(-c5ccccc5)cc5ccccc45)cc3)c3ccc(-n4c5ccccc5c5ccccc54)cc3)c2)cc1 ; c1ccc(-c2cc(-c3ccc(N(c4ccccc4)c4ccc(-c5ccc(-n6c7ccccc7c7ccccc76)cc5)cc4)cc3)c3ccccc3c2)cc1] 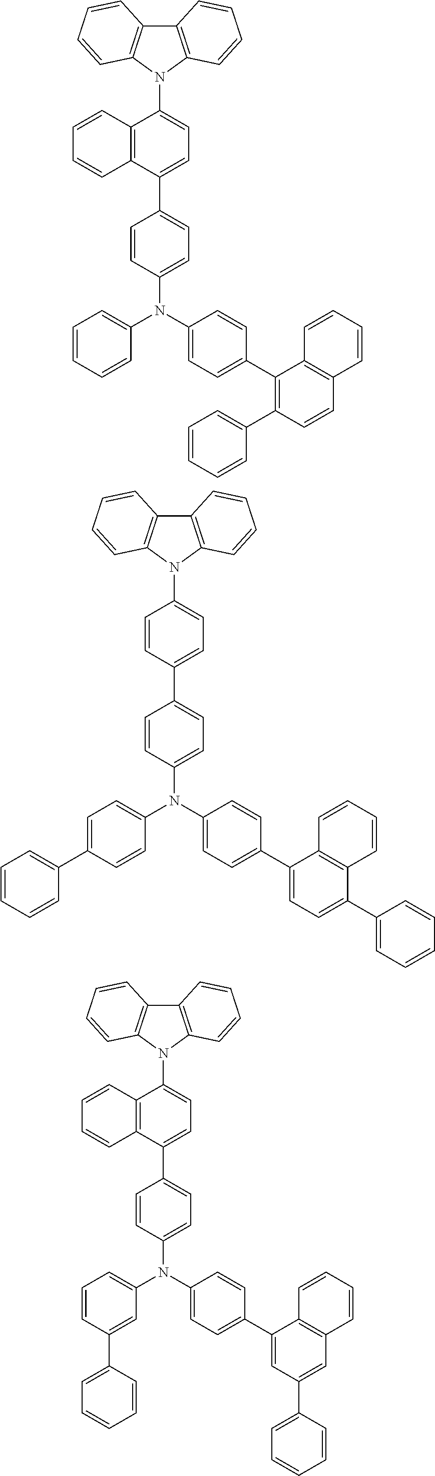 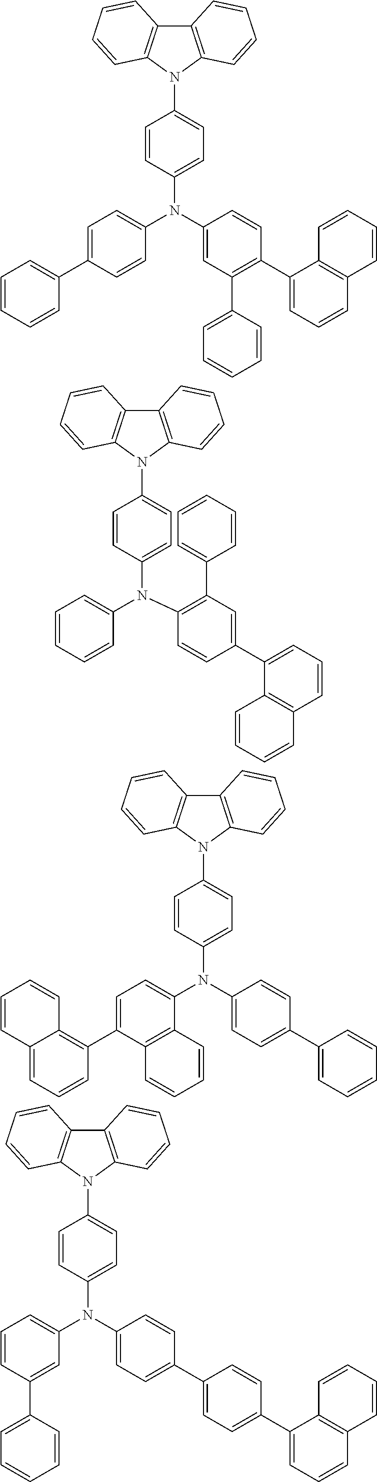 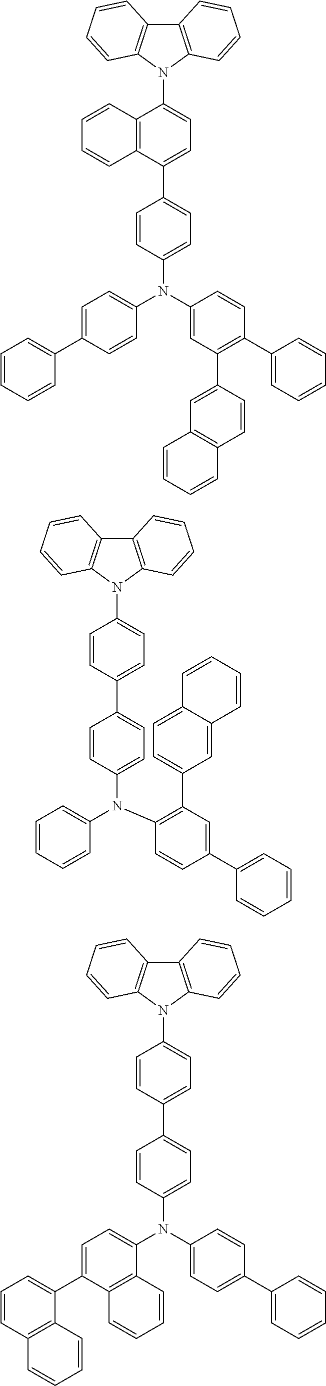 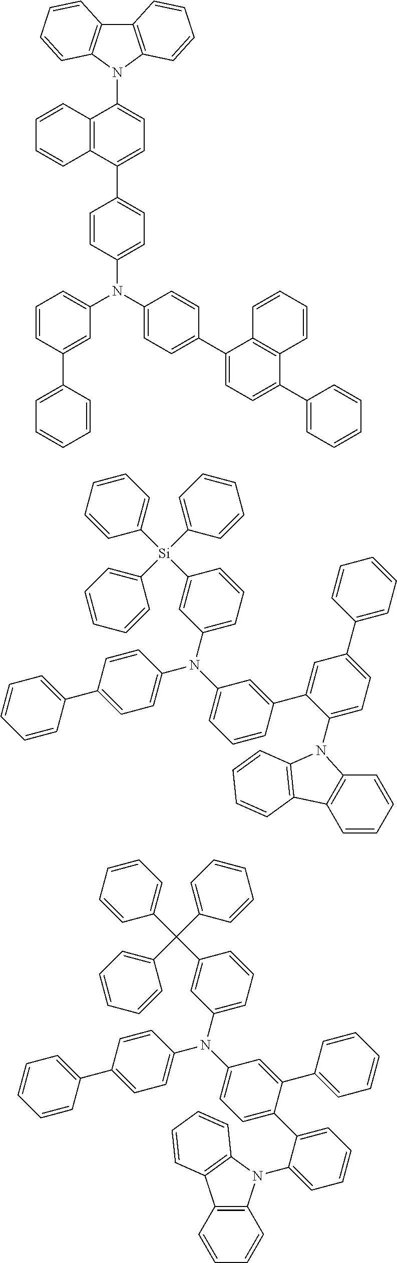 [SMILES: c1ccc(-c2cccc(N(c3ccc(-c4ccc(-c5ccccc5)c5ccccc45)cc3)c3ccc(-c4ccc(-n5c6ccccc6c6ccccc65)c5ccccc45)cc3)c2)cc1 ; c1ccc(-c2ccc(N(c3cccc(-c4cc(-c5ccccc5)ccc4-n4c5ccccc5c5ccccc54)c3)c3cccc([Si](c4ccccc4)(c4ccccc4)c4ccccc4)c3)cc2)cc1 ; c1ccc(-c2ccc(N(c3cccc(C(c4ccccc4)(c4ccccc4)c4ccccc4)c3)c3ccc(-c4ccccc4-n4c5ccccc5c5ccccc54)c(-c4ccccc4)c3)cc2)cc1] 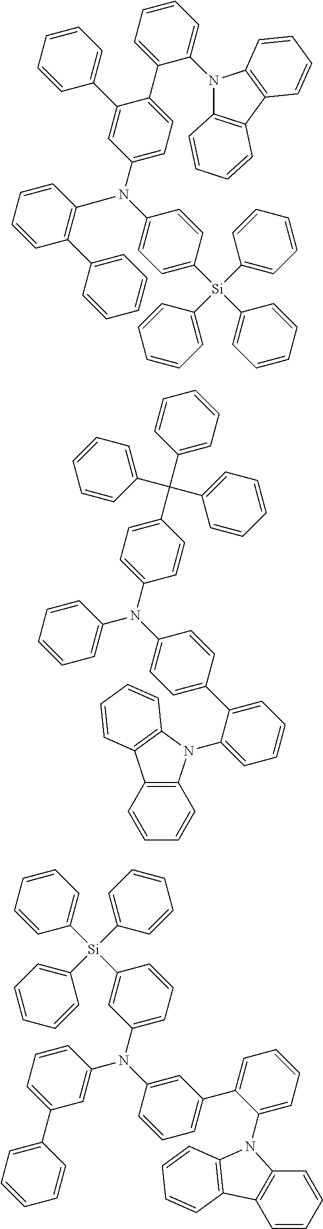 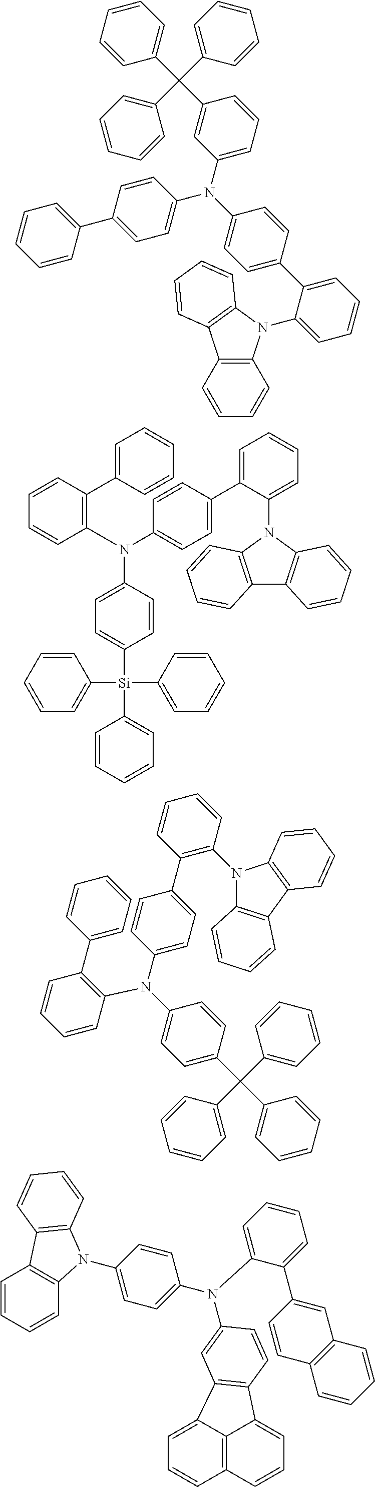 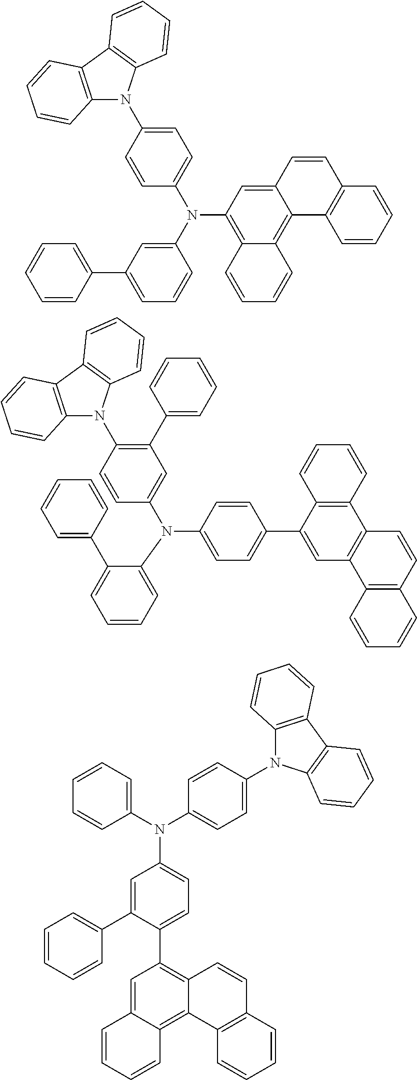 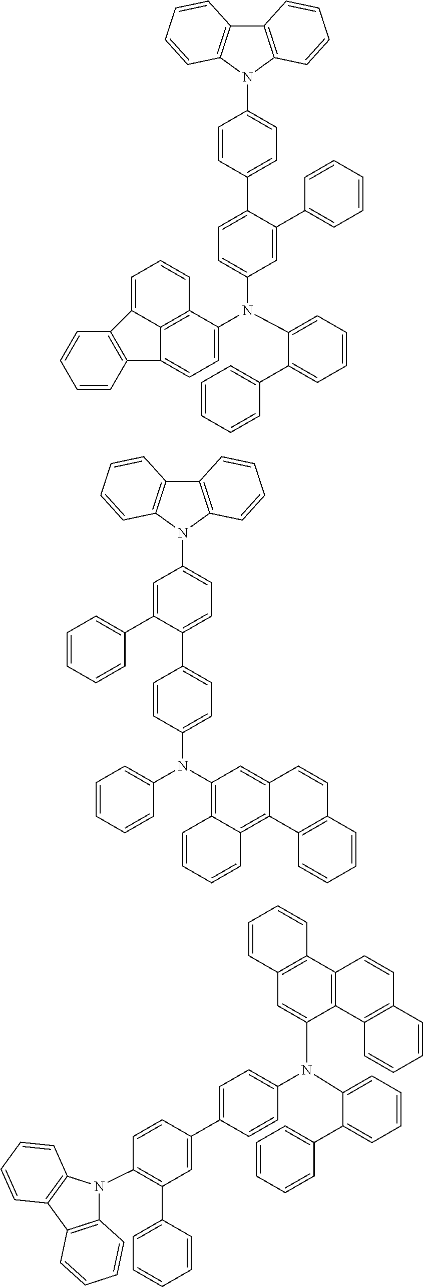 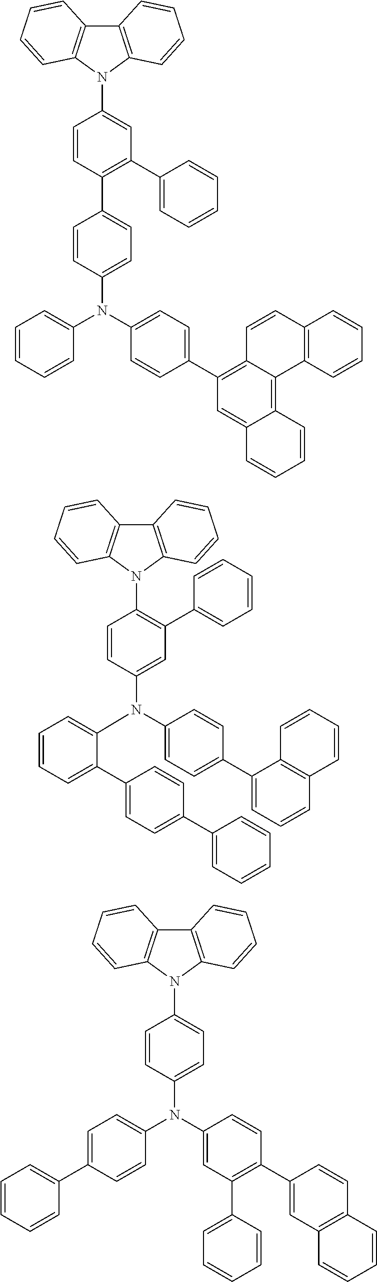 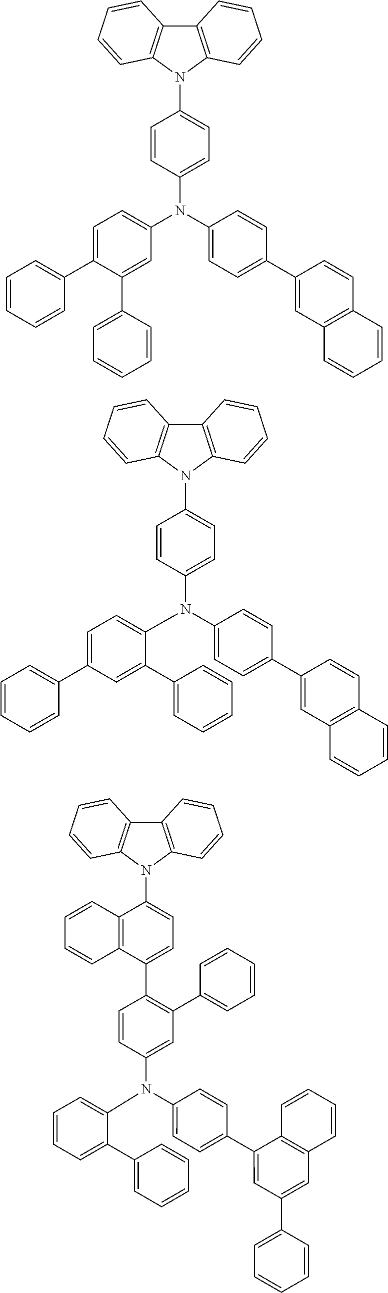 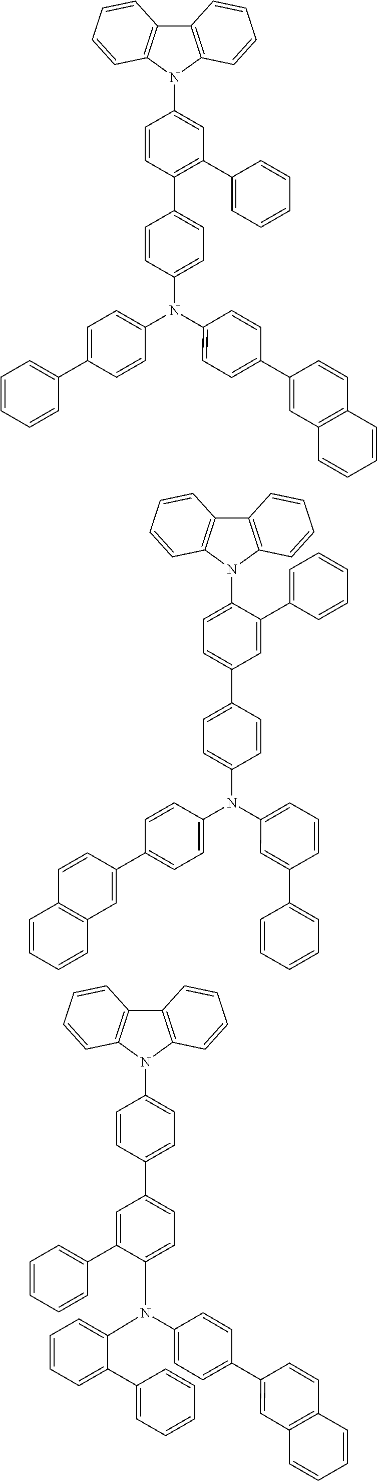 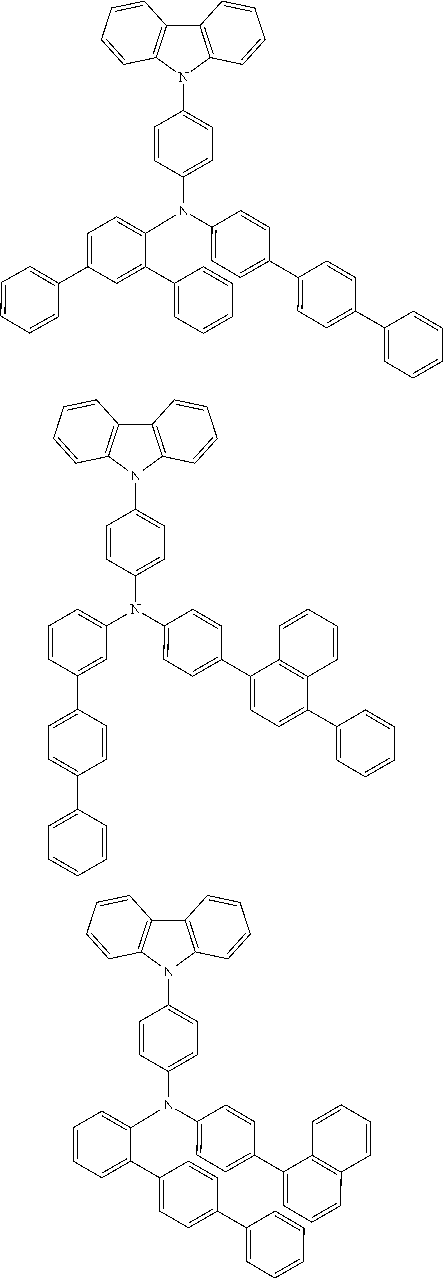 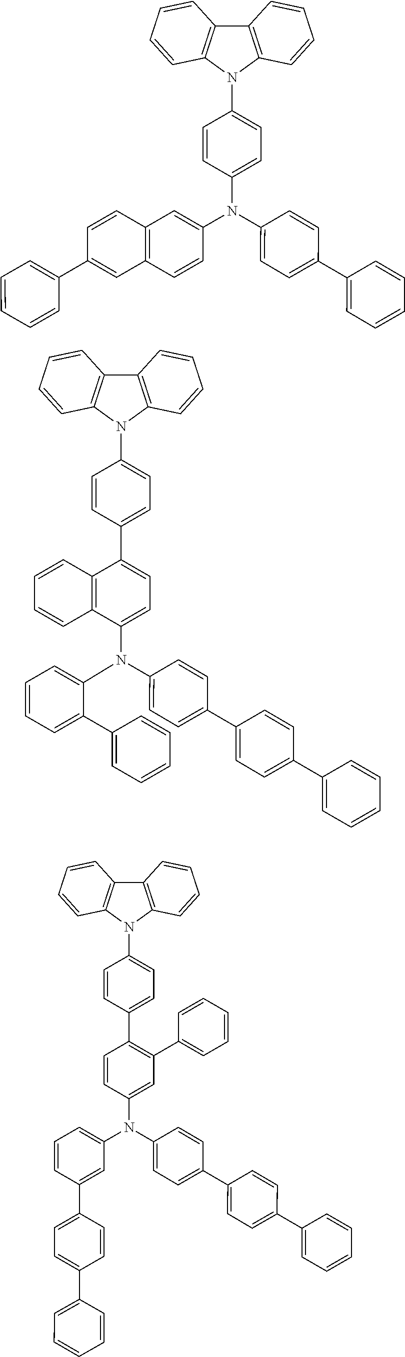 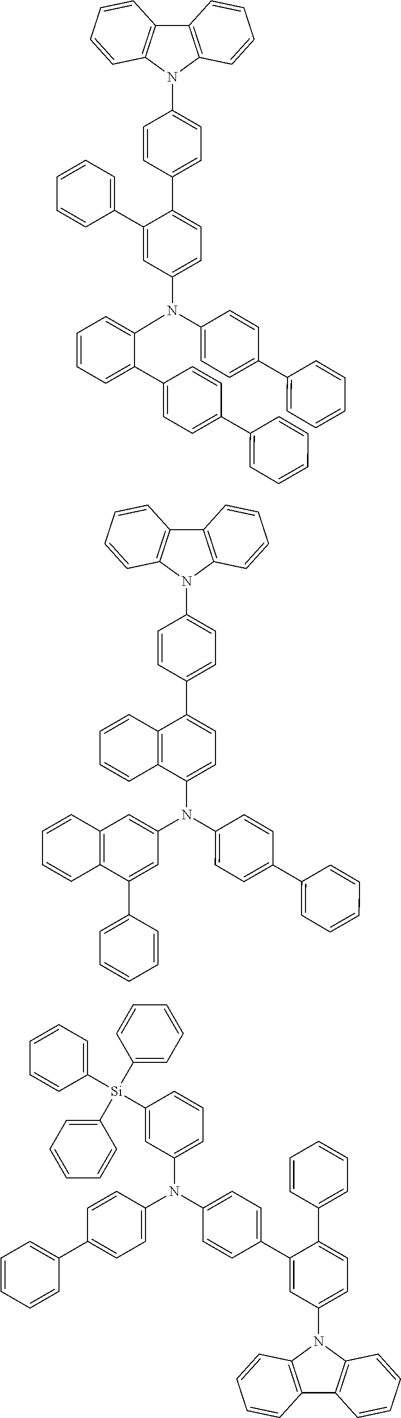 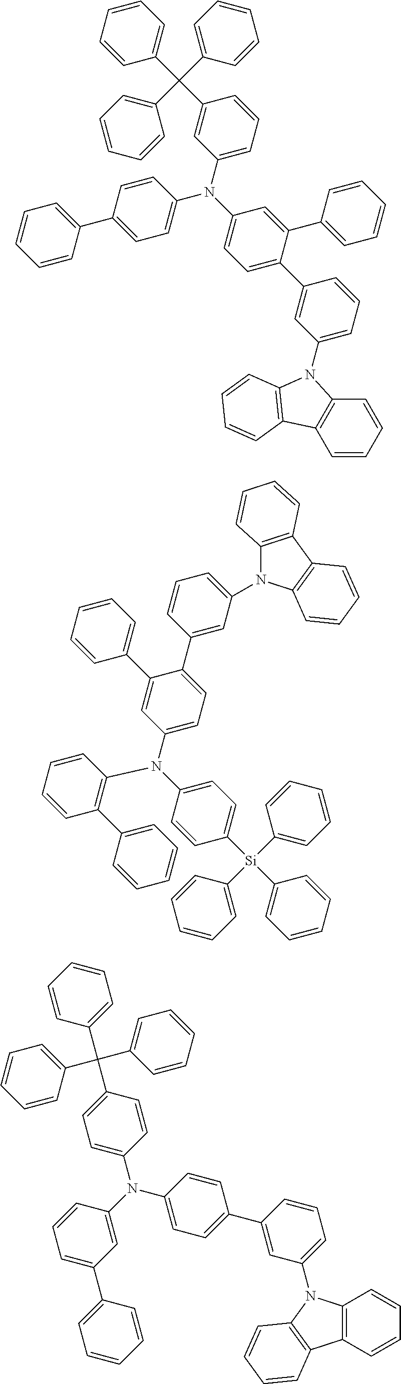 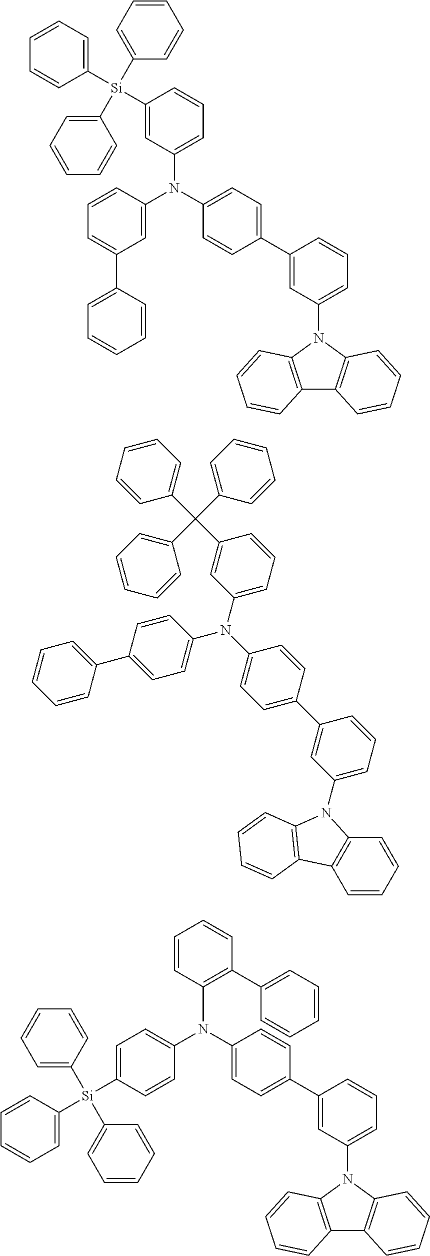 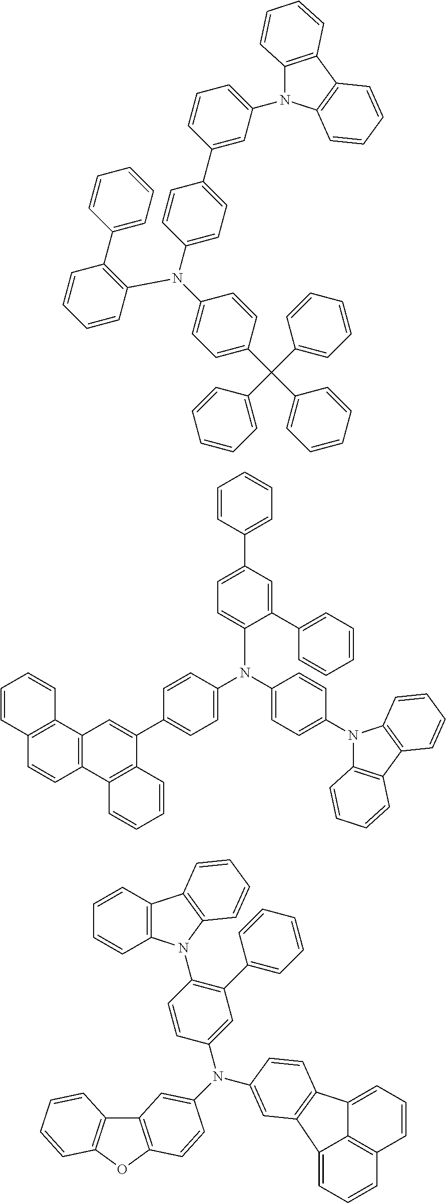 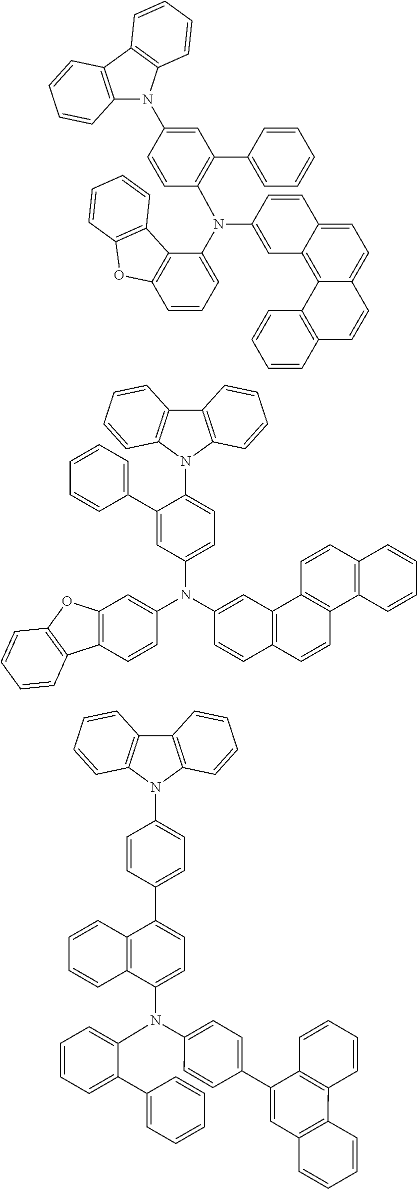 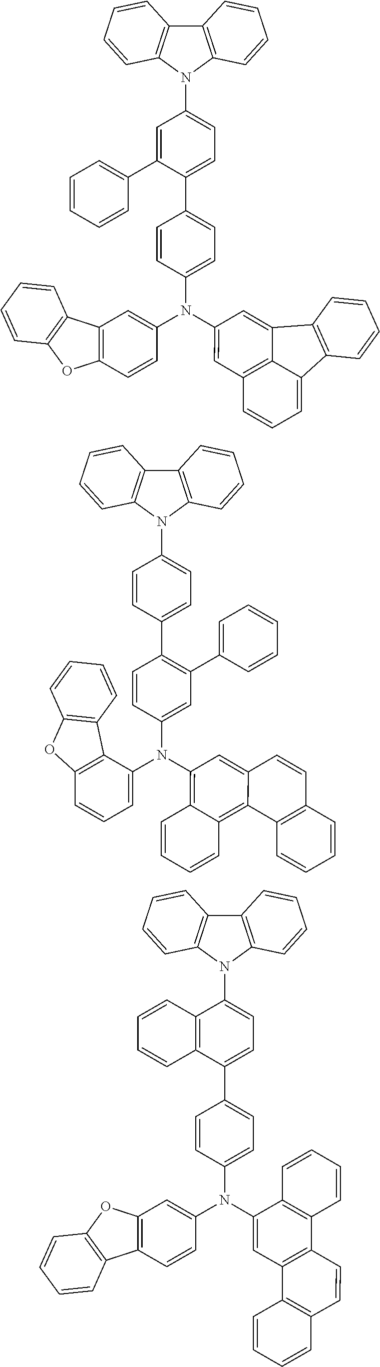 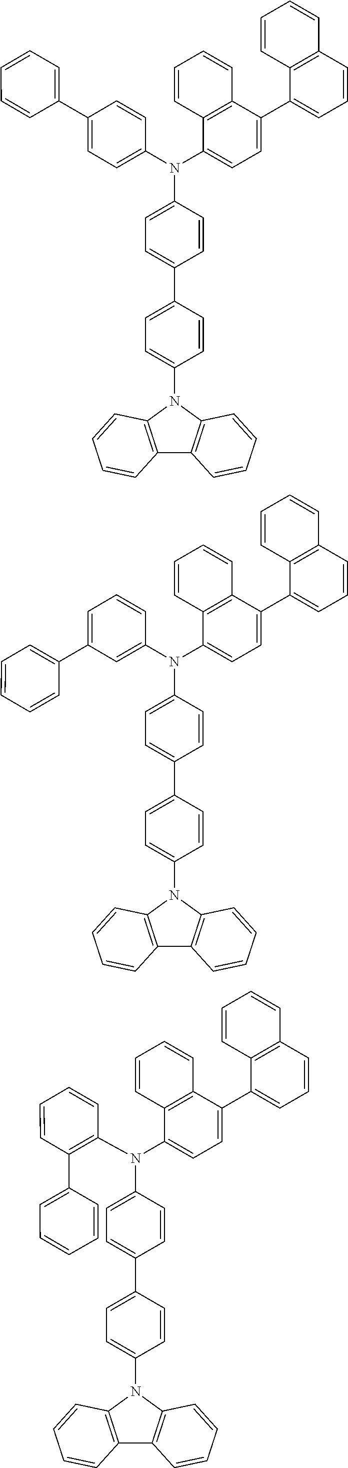 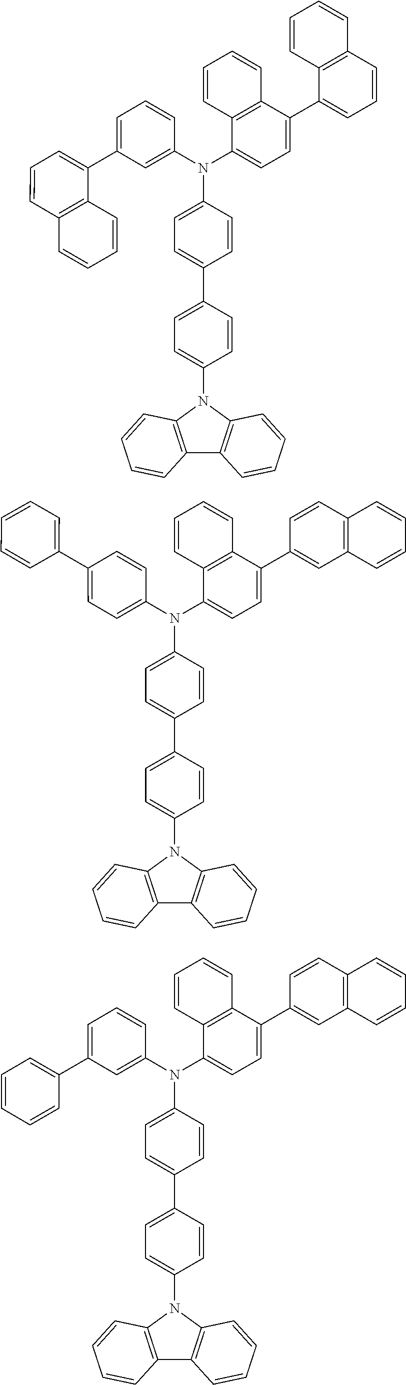 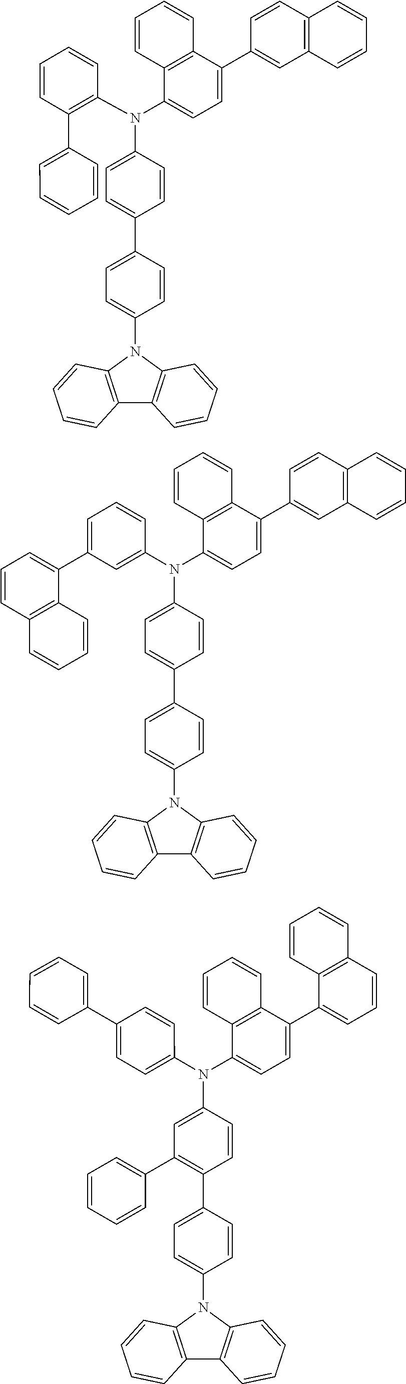 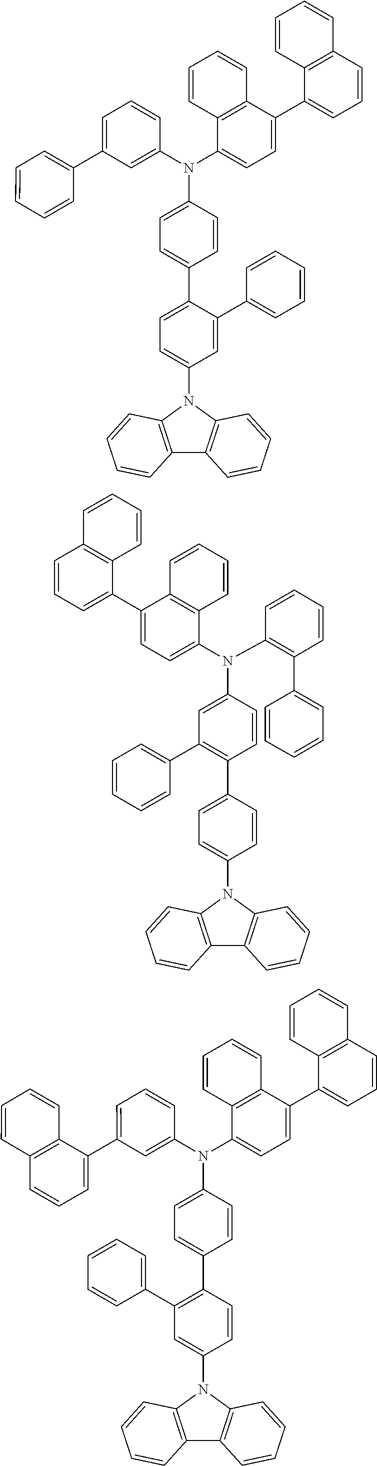 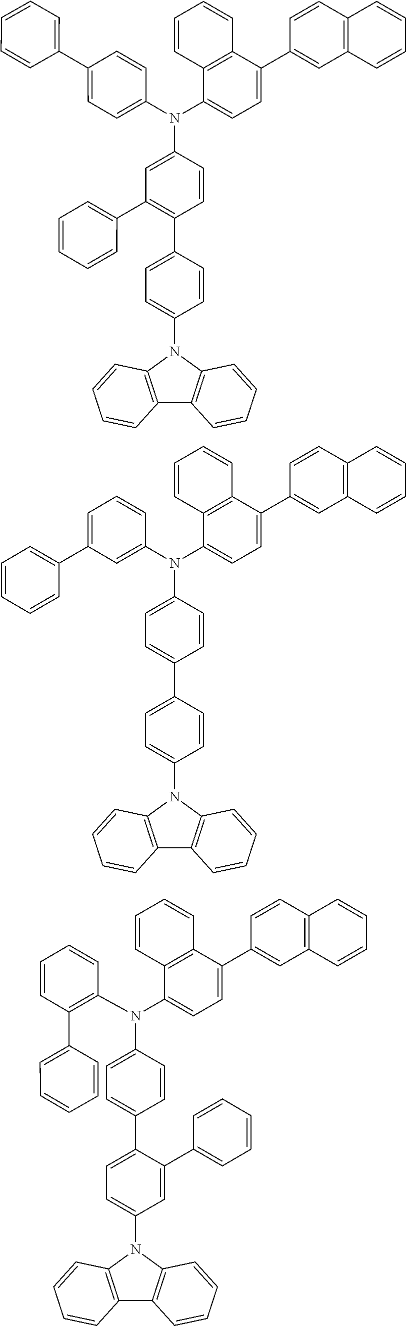 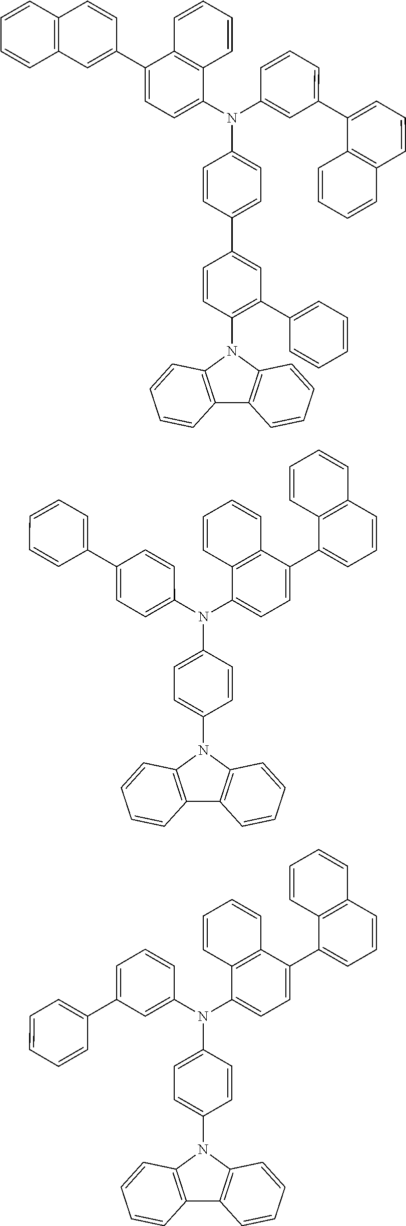 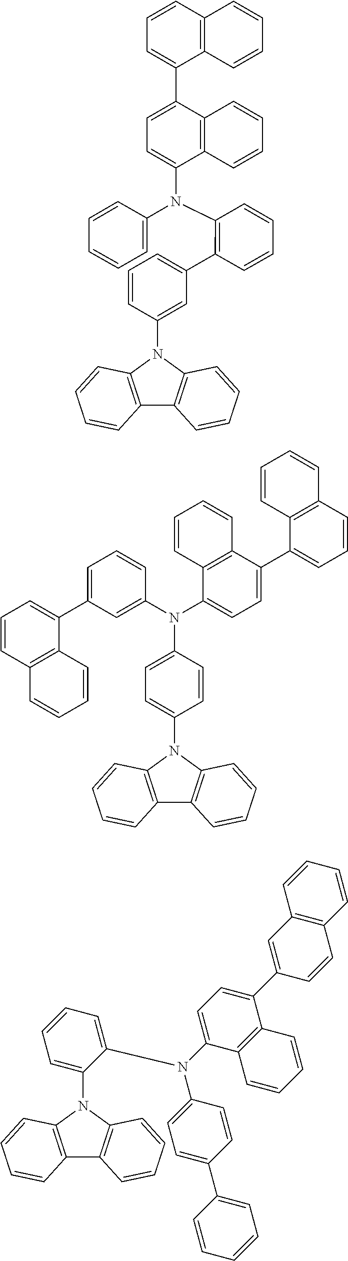 [SMILES: c1ccc(N(c2ccccc2-c2cccc(-n3c4ccccc4c4ccccc43)c2)c2ccc(-c3cccc4ccccc34)c3ccccc23)cc1 ; c1cc(-c2cccc3ccccc23)cc(N(c2ccc(-n3c4ccccc4c4ccccc43)cc2)c2ccc(-c3cccc4ccccc34)c3ccccc23)c1 ; c1ccc(-c2ccc(N(c3ccccc3-n3c4ccccc4c4ccccc43)c3ccc(-c4ccc5ccccc5c4)c4ccccc34)cc2)cc1] 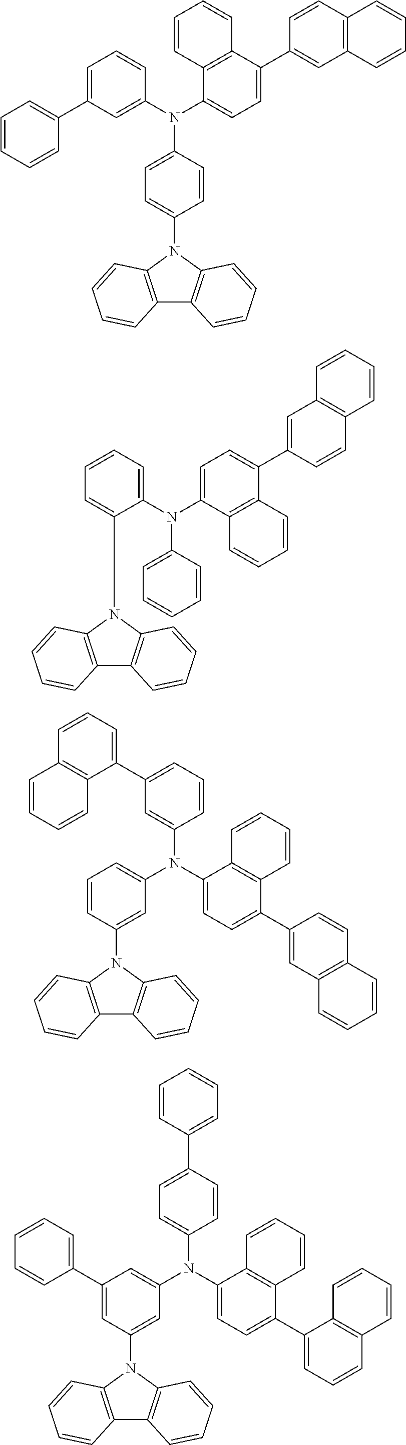 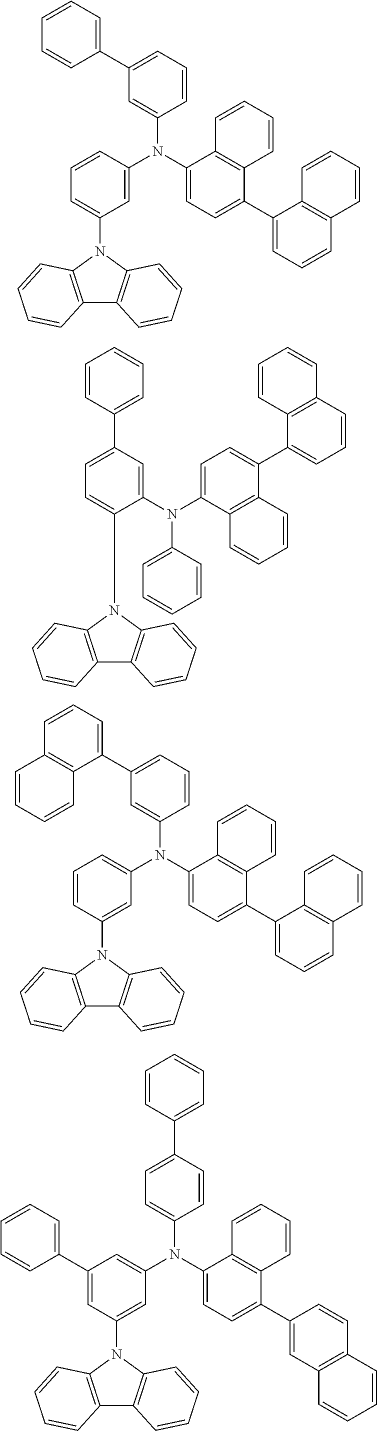 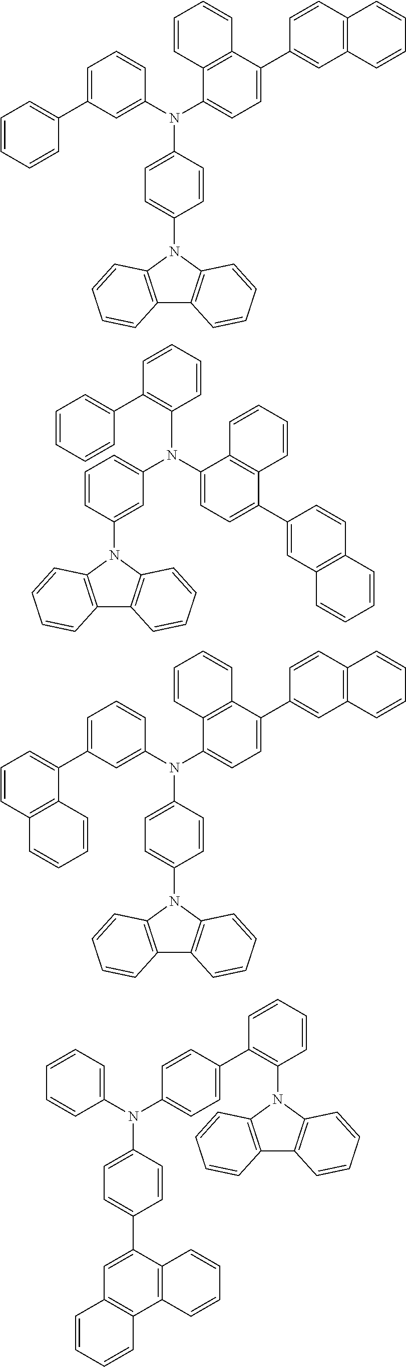 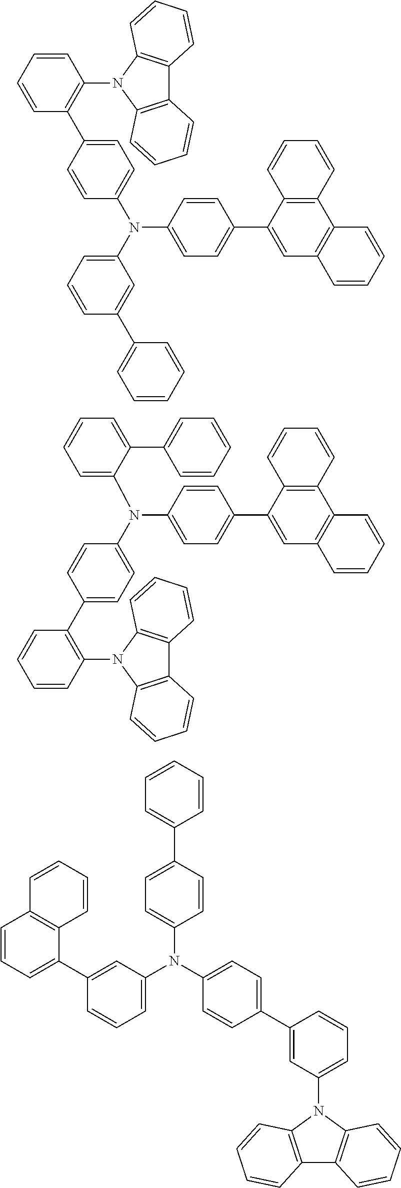 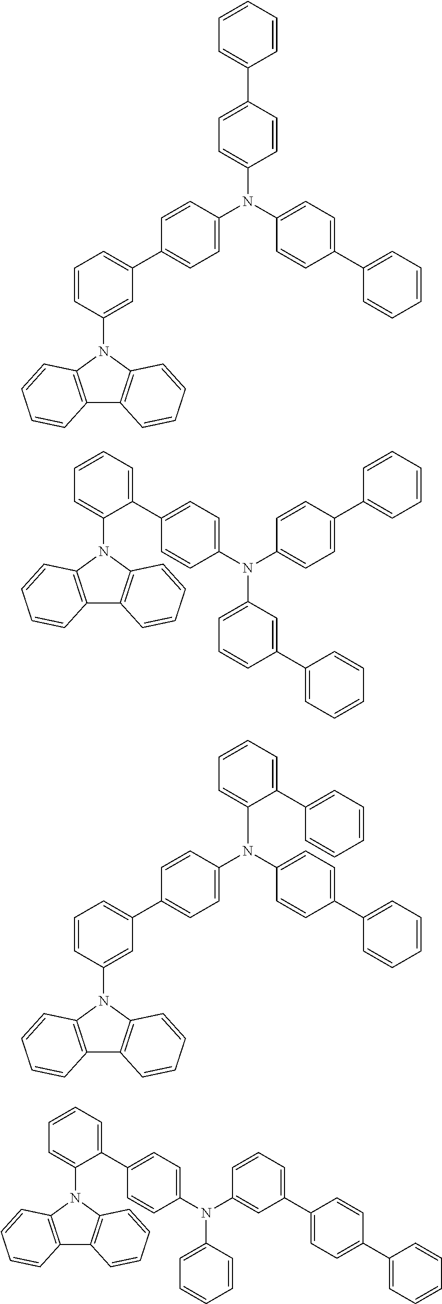 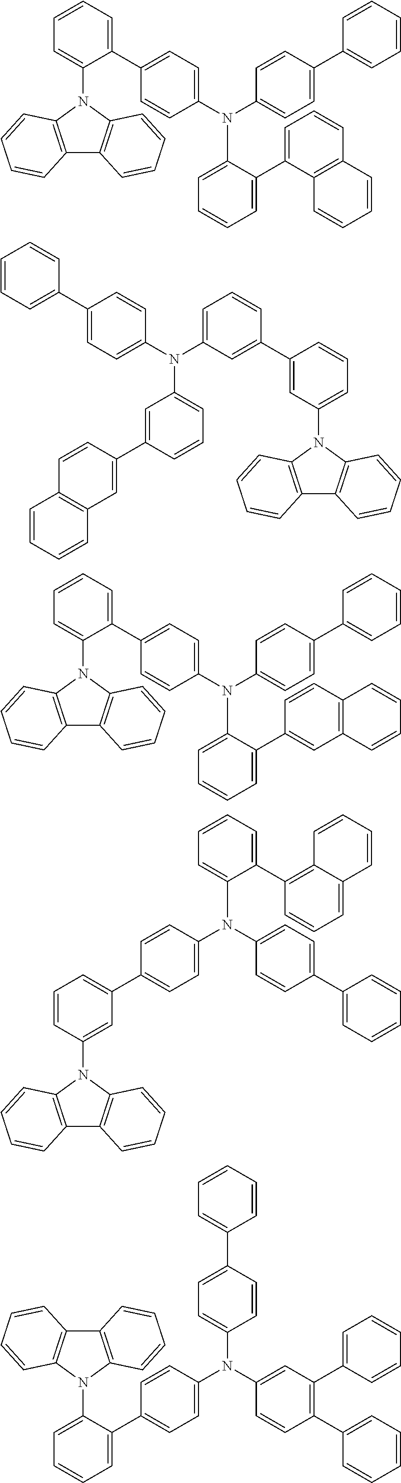 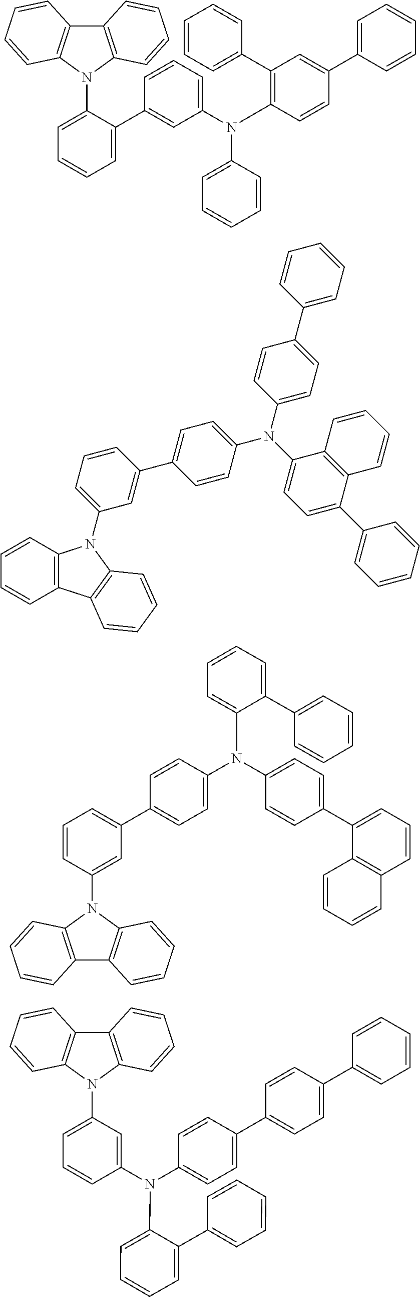 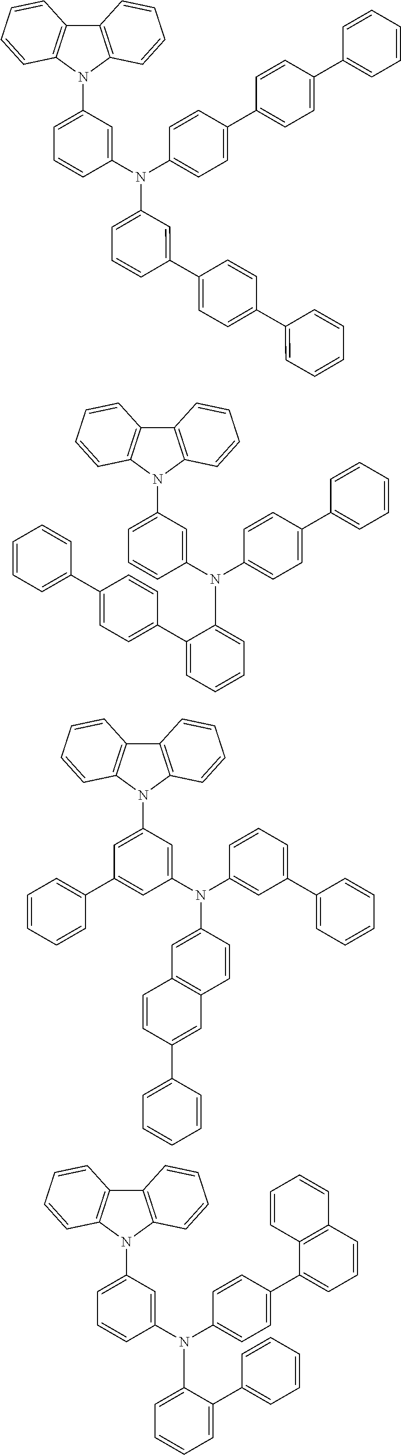 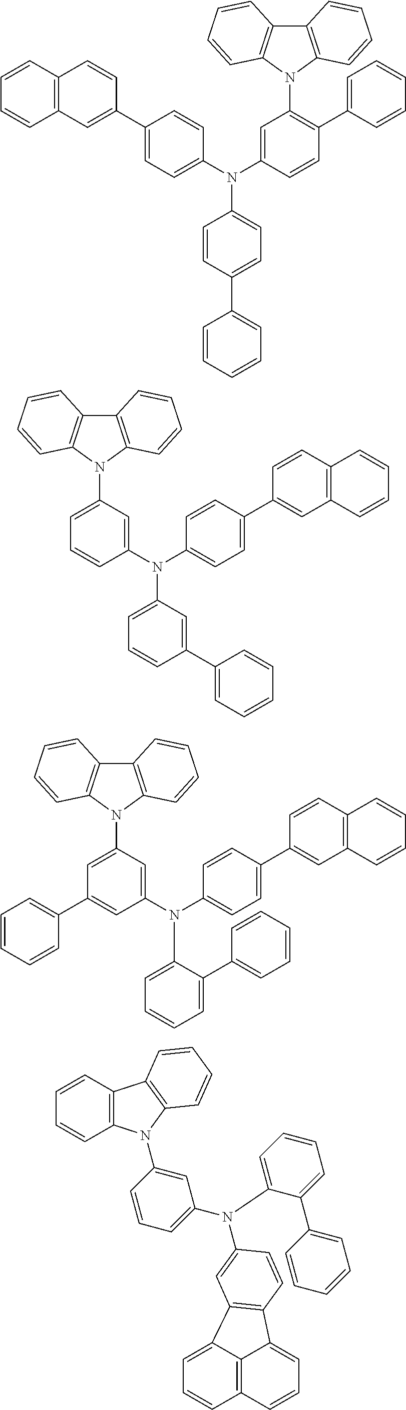 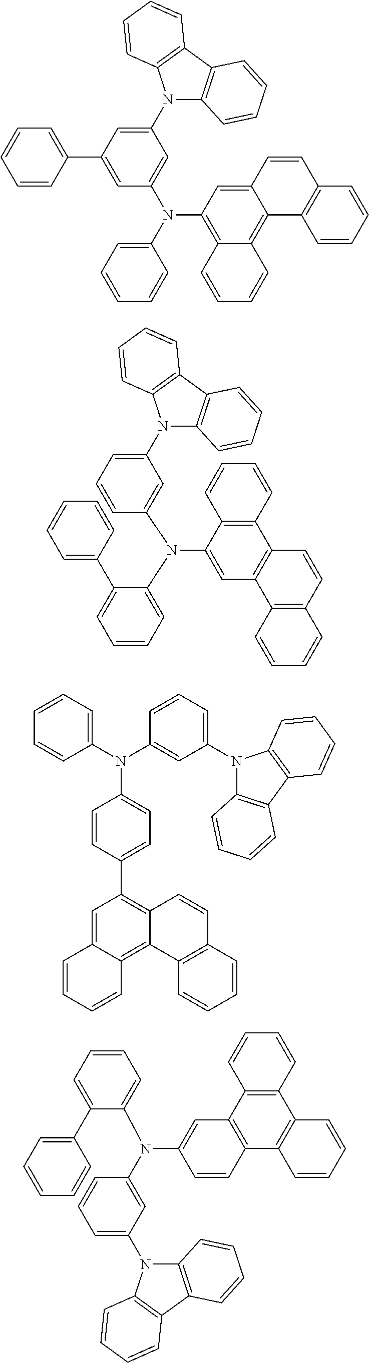 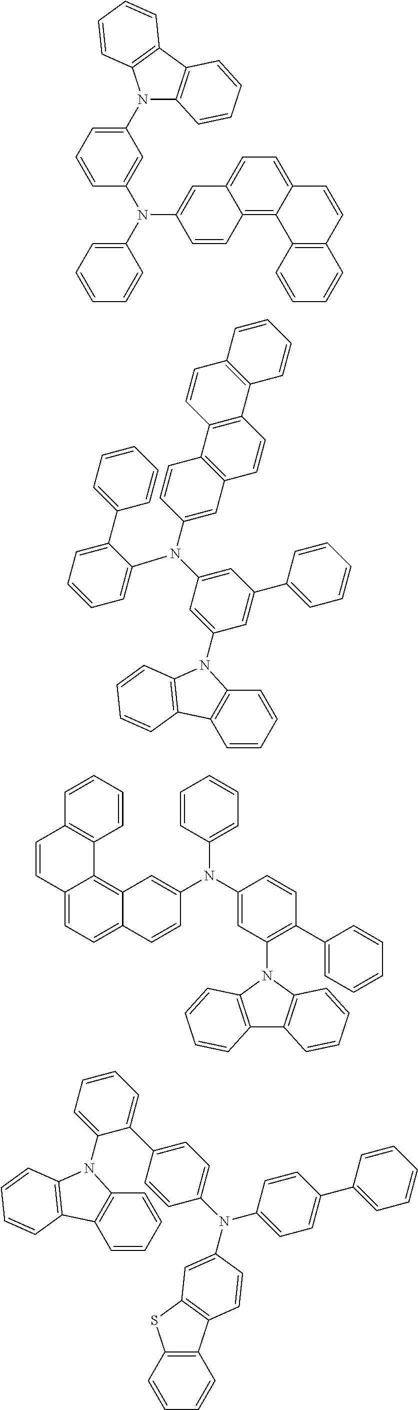 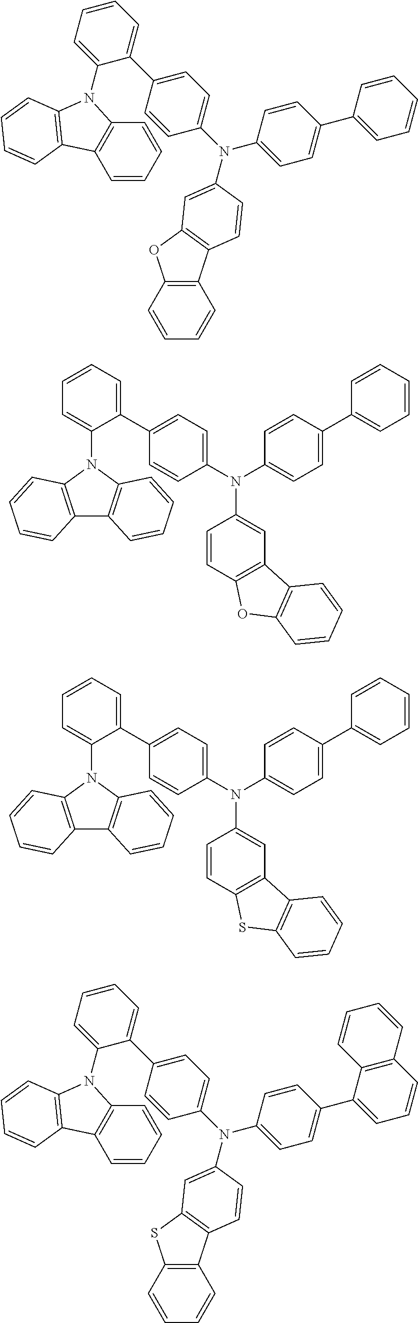 [SMILES: c1ccc(-c2ccc(N(c3ccc(-c4ccccc4-n4c5ccccc5c5ccccc54)cc3)c3ccc4c(c3)oc3ccccc34)cc2)cc1 ; c1ccc(-c2ccc(N(c3ccc(-c4ccccc4-n4c5ccccc5c5ccccc54)cc3)c3ccc4oc5ccccc5c4c3)cc2)cc1 ; c1ccc(-c2ccc(N(c3ccc(-c4ccccc4-n4c5ccccc5c5ccccc54)cc3)c3ccc4sc5ccccc5c4c3)cc2)cc1 ; c1ccc(-n2c3ccccc3c3ccccc32)c(-c2ccc(N(c3ccc(-c4cccc5ccccc45)cc3)c3ccc4c(c3)sc3ccccc34)cc2)c1] 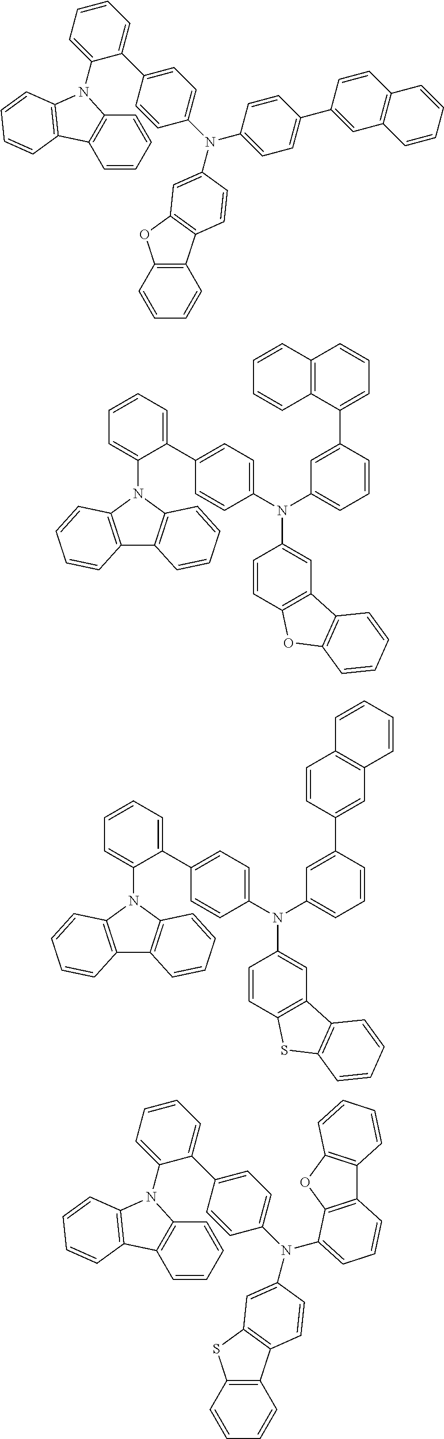 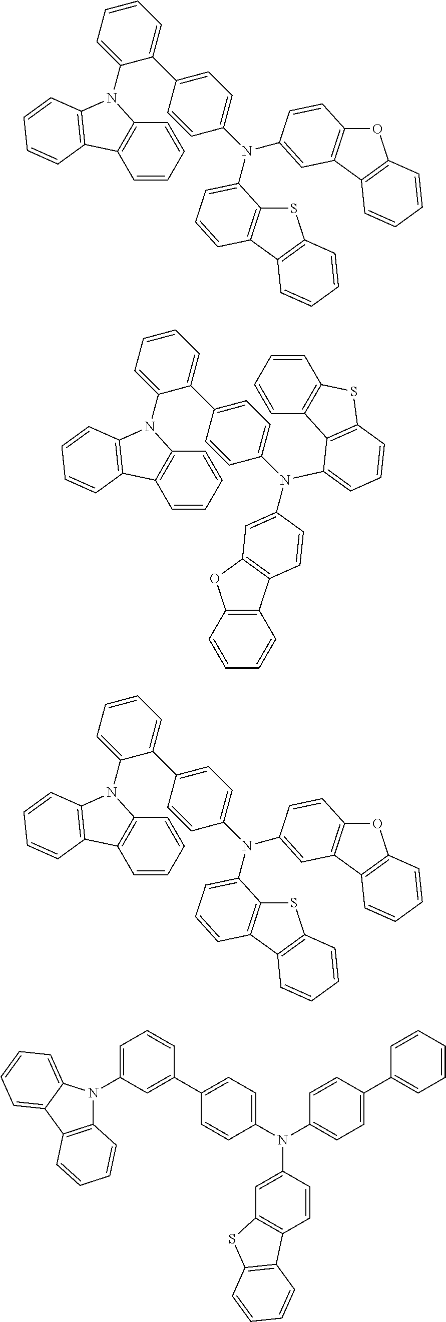 [SMILES: c1ccc(-n2c3ccccc3c3ccccc32)c(-c2ccc(N(c3ccc4oc5ccccc5c4c3)c3cccc4c3sc3ccccc34)cc2)c1 ; c1ccc(-n2c3ccccc3c3ccccc32)c(-c2ccc(N(c3ccc4c(c3)oc3ccccc34)c3cccc4sc5ccccc5c34)cc2)c1 ; c1ccc(-n2c3ccccc3c3ccccc32)c(-c2ccc(N(c3ccc4oc5ccccc5c4c3)c3cccc4c3sc3ccccc34)cc2)c1 ; c1ccc(-c2ccc(N(c3ccc(-c4cccc(-n5c6ccccc6c6ccccc65)c4)cc3)c3ccc4c(c3)sc3ccccc34)cc2)cc1] 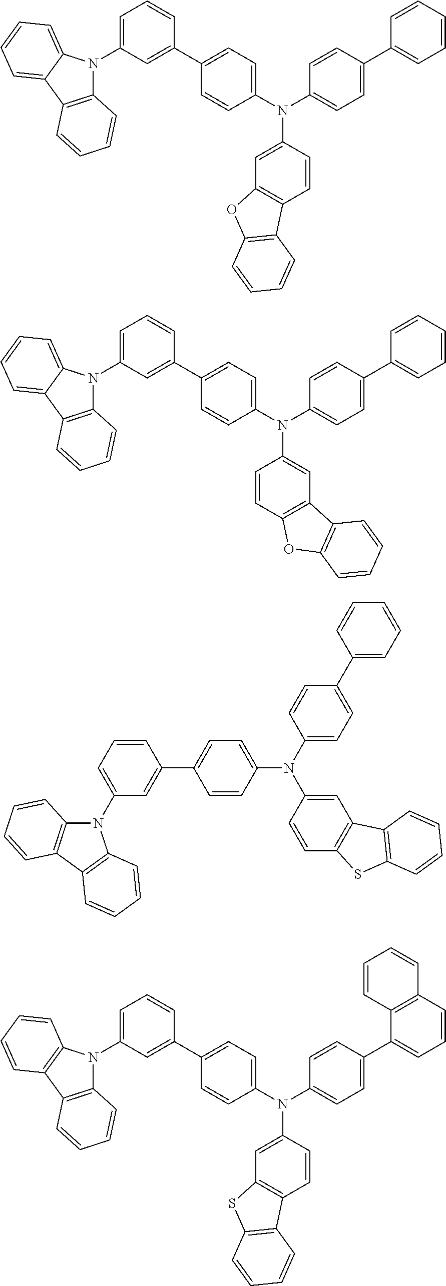 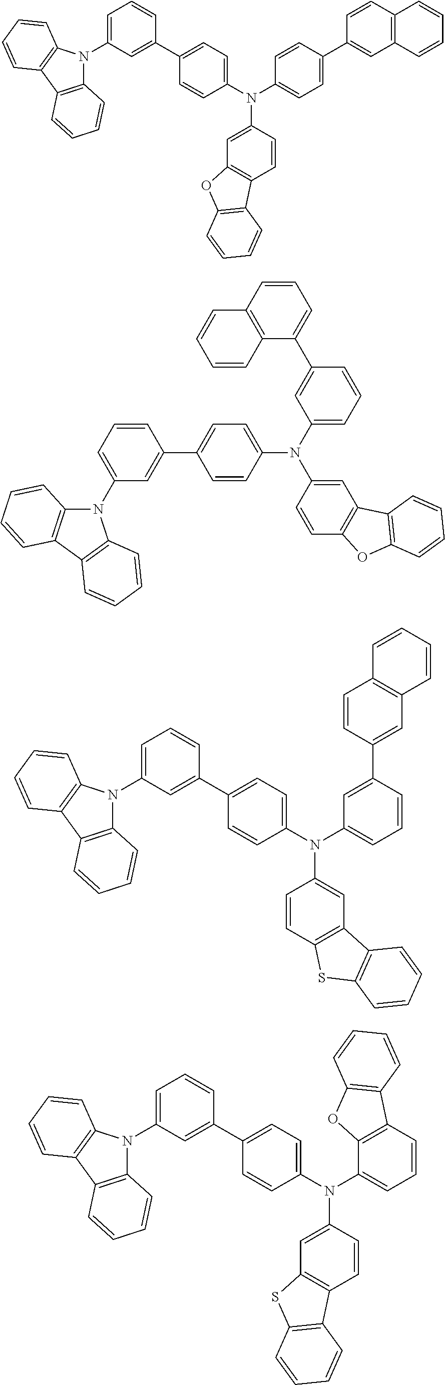 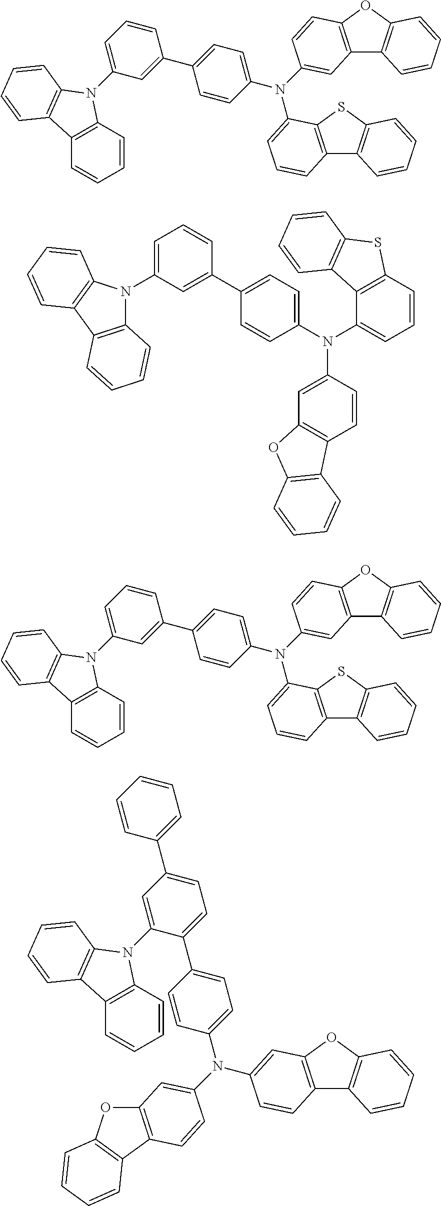 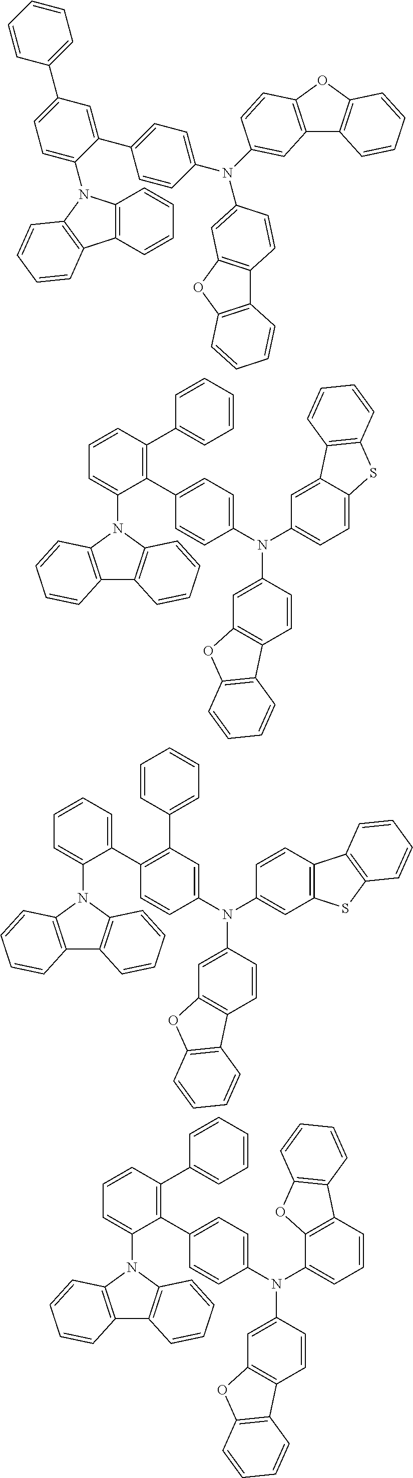 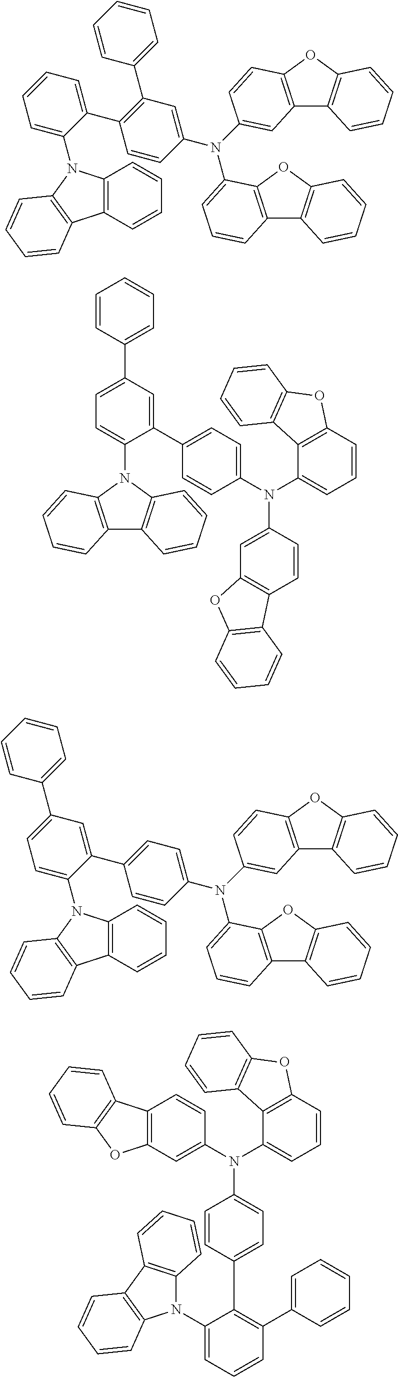 [SMILES: c1ccc(-c2cc(N(c3ccc4oc5ccccc5c4c3)c3cccc4c3oc3ccccc34)ccc2-c2ccccc2-n2c3ccccc3c3ccccc32)cc1 ; c1ccc(-c2ccc(-n3c4ccccc4c4ccccc43)c(-c3ccc(N(c4ccc5c(c4)oc4ccccc45)c4cccc5oc6ccccc6c45)cc3)c2)cc1 ; c1ccc(-c2ccc(-n3c4ccccc4c4ccccc43)c(-c3ccc(N(c4ccc5oc6ccccc6c5c4)c4cccc5c4oc4ccccc45)cc3)c2)cc1 ; c1ccc(-c2cccc(-n3c4ccccc4c4ccccc43)c2-c2ccc(N(c3ccc4c(c3)oc3ccccc34)c3cccc4oc5ccccc5c34)cc2)cc1] 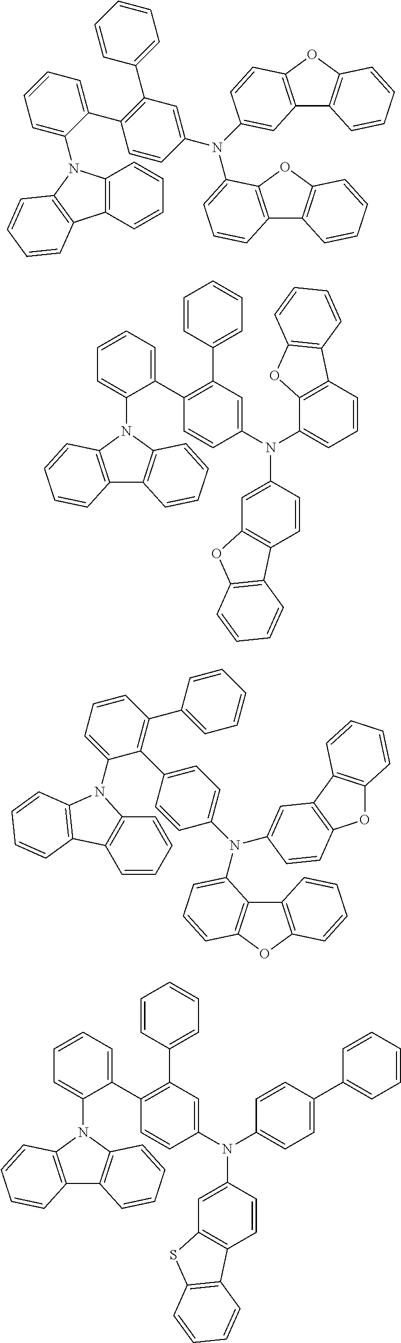 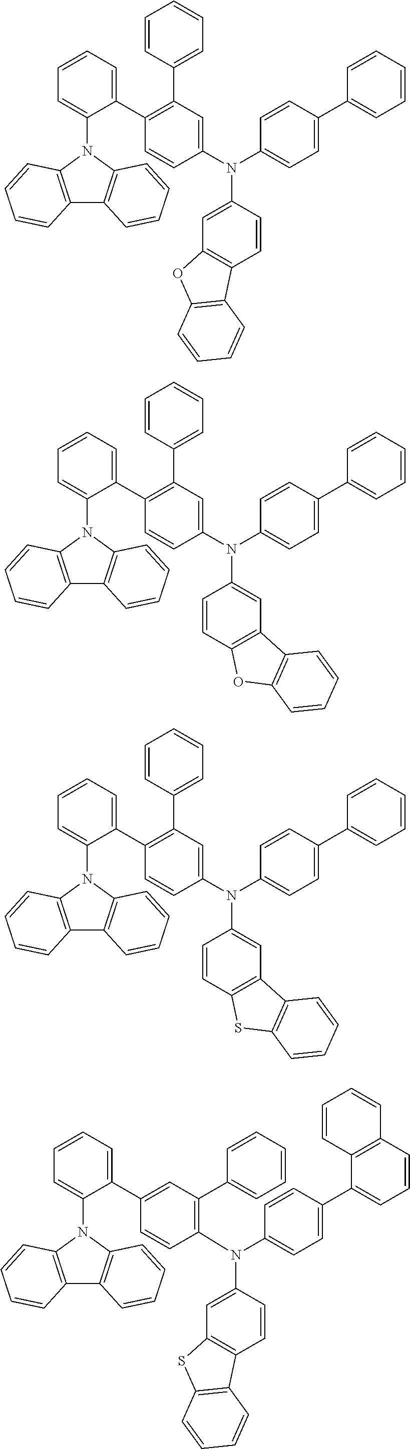 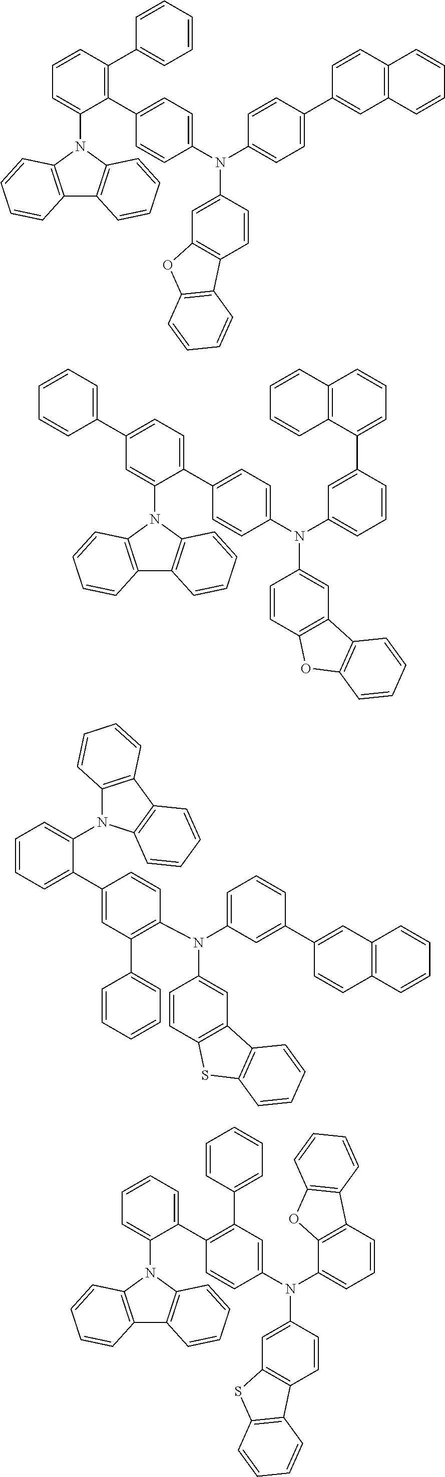 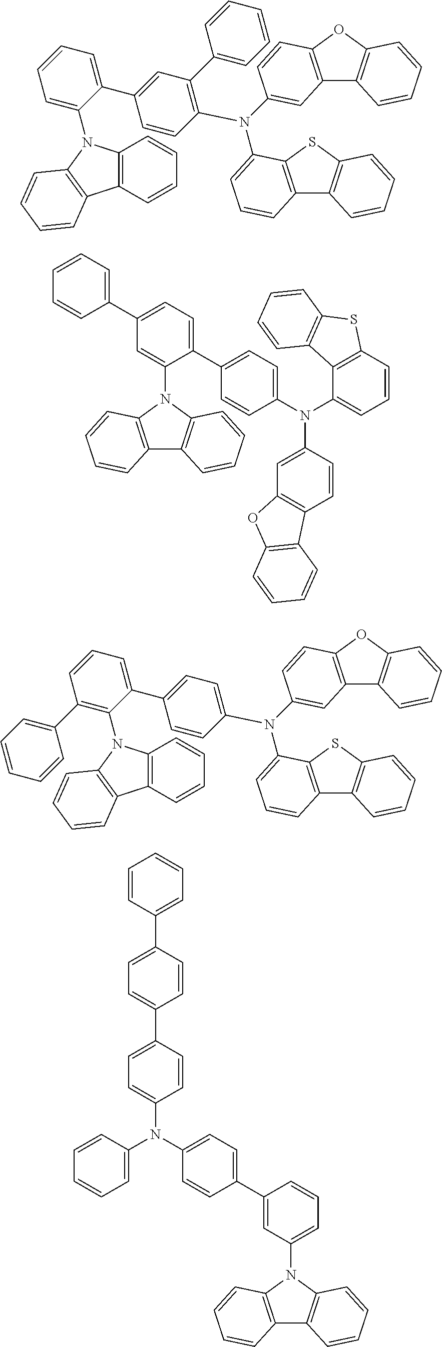 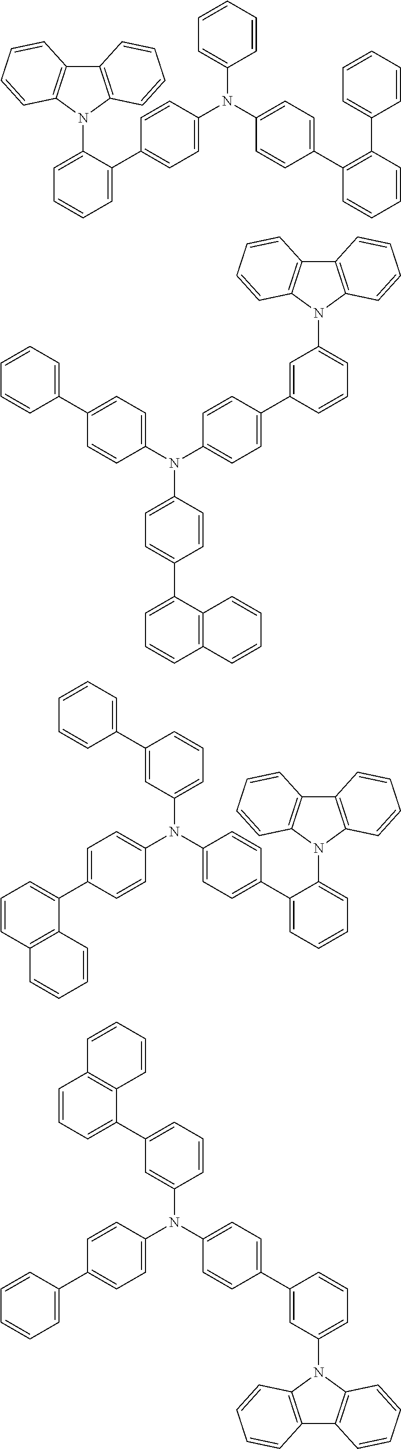 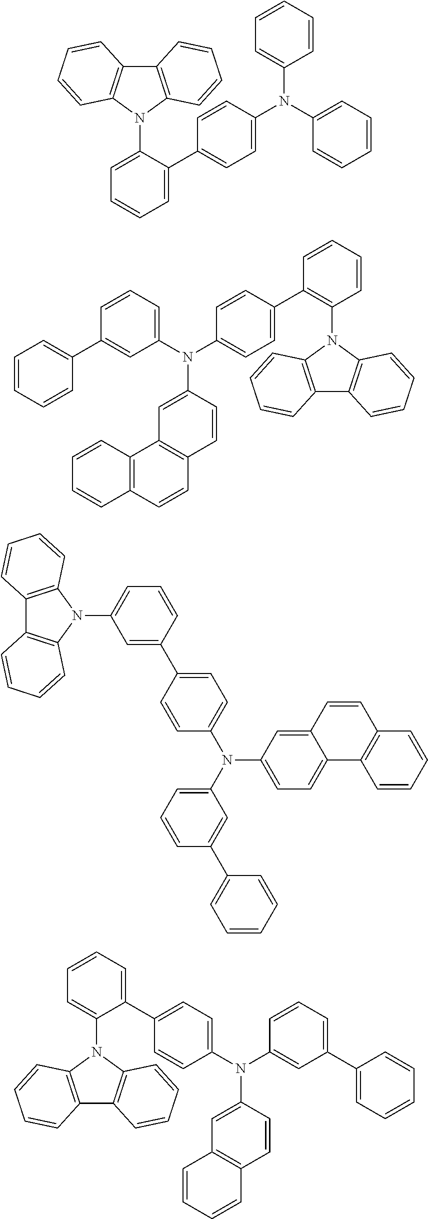 [SMILES: c1ccc(N(c2ccccc2)c2ccc(-c3ccccc3-n3c4ccccc4c4ccccc43)cc2)cc1 ; c1ccc(-c2cccc(N(c3ccc(-c4ccccc4-n4c5ccccc5c5ccccc54)cc3)c3ccc4ccc5ccccc5c4c3)c2)cc1 ; c1ccc(-c2cccc(N(c3ccc(-c4cccc(-n5c6ccccc6c6ccccc65)c4)cc3)c3ccc4c(ccc5ccccc54)c3)c2)cc1 ; c1ccc(-c2cccc(N(c3ccc(-c4ccccc4-n4c5ccccc5c5ccccc54)cc3)c3ccc4ccccc4c3)c2)cc1] 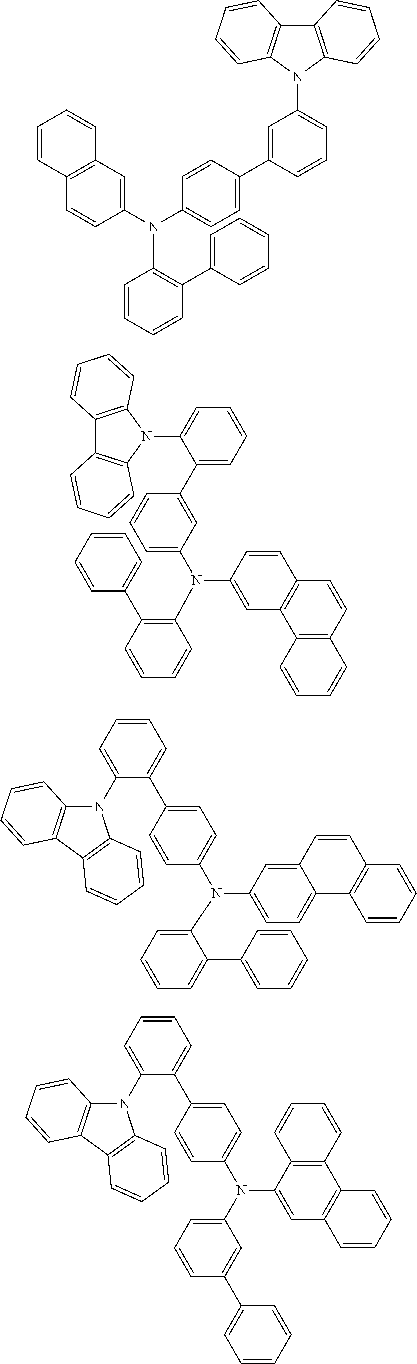 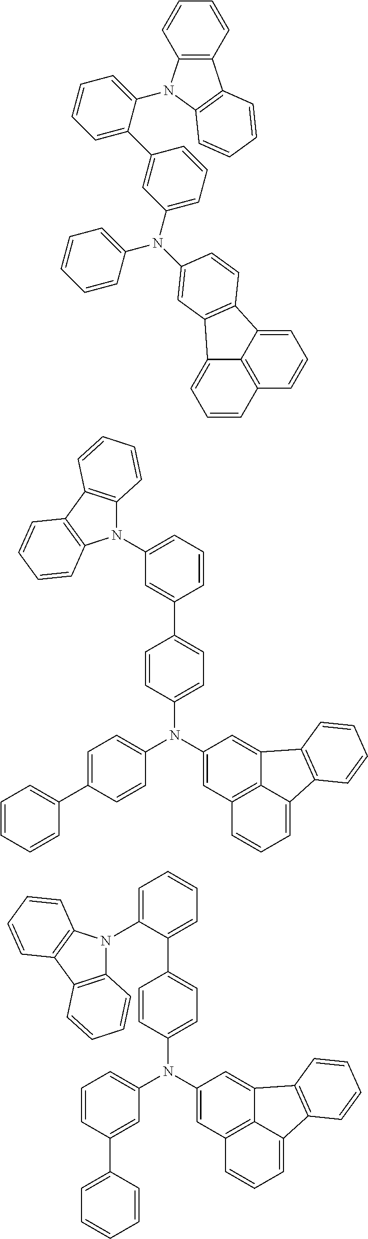 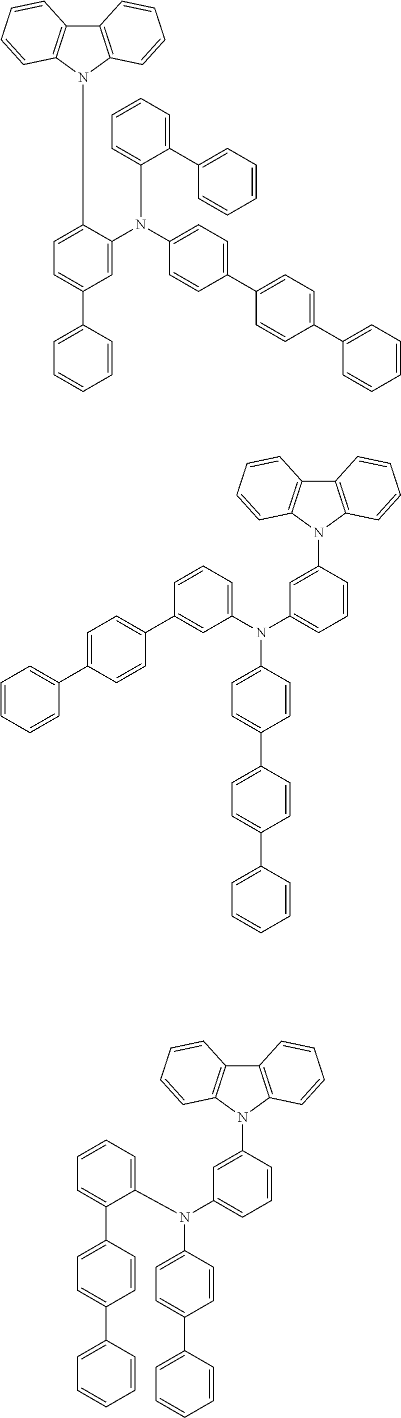 [SMILES: c1ccc(-c2ccc(-c3ccc(N(c4ccccc4-c4ccccc4)c4cc(-c5ccccc5)ccc4-n4c5ccccc5c5ccccc54)cc3)cc2)cc1 ; c1ccc(-c2ccc(-c3ccc(N(c4cccc(-c5ccc(-c6ccccc6)cc5)c4)c4cccc(-n5c6ccccc6c6ccccc65)c4)cc3)cc2)cc1 ; c1ccc(-c2ccc(-c3ccccc3N(c3ccc(-c4ccccc4)cc3)c3cccc(-n4c5ccccc5c5ccccc54)c3)cc2)cc1] 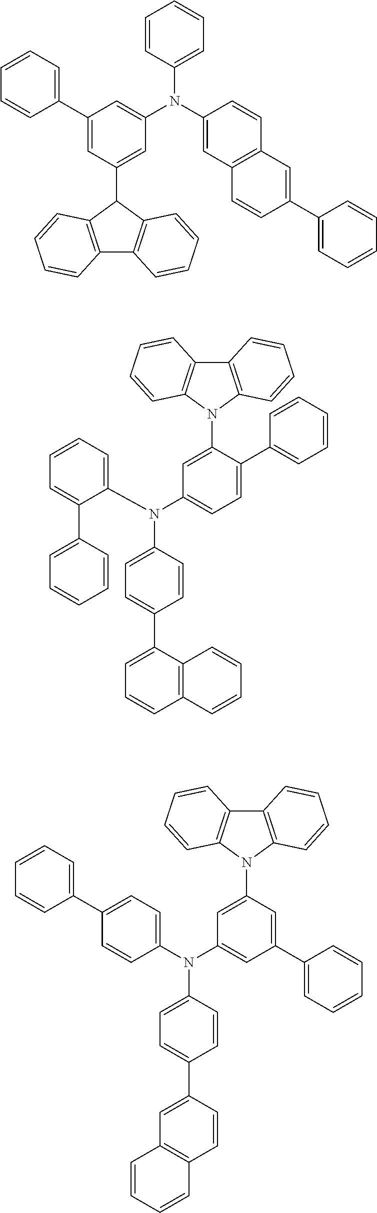 [SMILES: c1ccc(-c2cc(C3c4ccccc4-c4ccccc43)cc(N(c3ccccc3)c3ccc4cc(-c5ccccc5)ccc4c3)c2)cc1 ; c1ccc(-c2ccccc2N(c2ccc(-c3cccc4ccccc34)cc2)c2ccc(-c3ccccc3)c(-n3c4ccccc4c4ccccc43)c2)cc1 ; c1ccc(-c2ccc(N(c3ccc(-c4ccc5ccccc5c4)cc3)c3cc(-c4ccccc4)cc(-n4c5ccccc5c5ccccc54)c3)cc2)cc1] 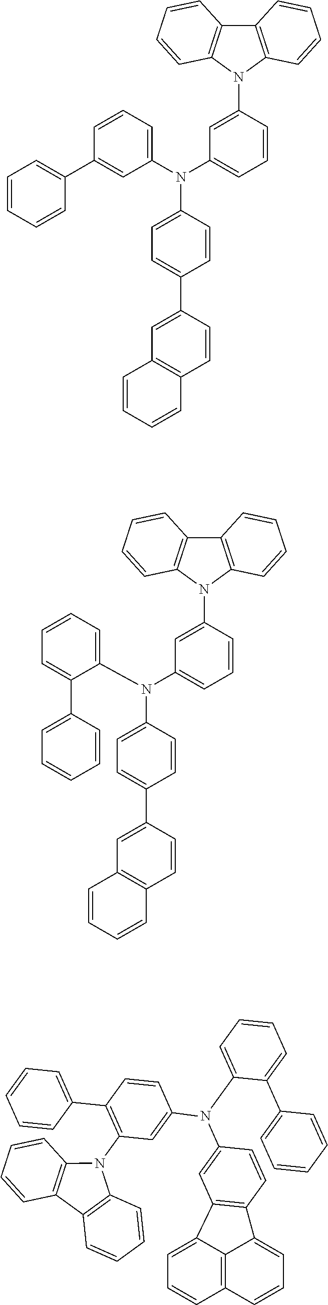 [SMILES: c1ccc(-c2cccc(N(c3ccc(-c4ccc5ccccc5c4)cc3)c3cccc(-n4c5ccccc5c5ccccc54)c3)c2)cc1 ; c1ccc(-c2ccccc2N(c2ccc(-c3ccc4ccccc4c3)cc2)c2cccc(-n3c4ccccc4c4ccccc43)c2)cc1 ; c1ccc(-c2ccccc2N(c2ccc3c(c2)-c2cccc4cccc-3c24)c2ccc(-c3ccccc3)c(-n3c4ccccc4c4ccccc43)c2)cc1] 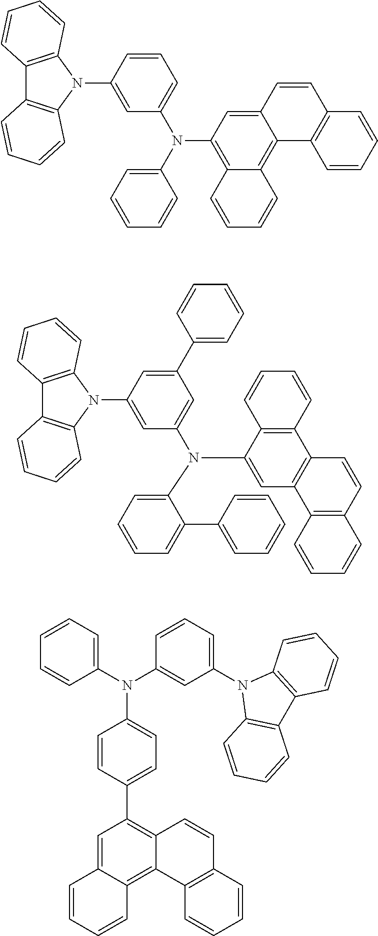 [SMILES: c1ccc(N(c2cccc(-n3c4ccccc4c4ccccc43)c2)c2cc3ccc4ccccc4c3c3ccccc23)cc1 ; c1ccc(-c2cc(N(c3ccccc3-c3ccccc3)c3cc4c5ccccc5ccc4c4ccccc34)cc(-n3c4ccccc4c4ccccc43)c2)cc1 ; c1ccc(N(c2ccc(-c3cc4ccccc4c4c3ccc3ccccc34)cc2)c2cccc(-n3c4ccccc4c4ccccc43)c2)cc1] 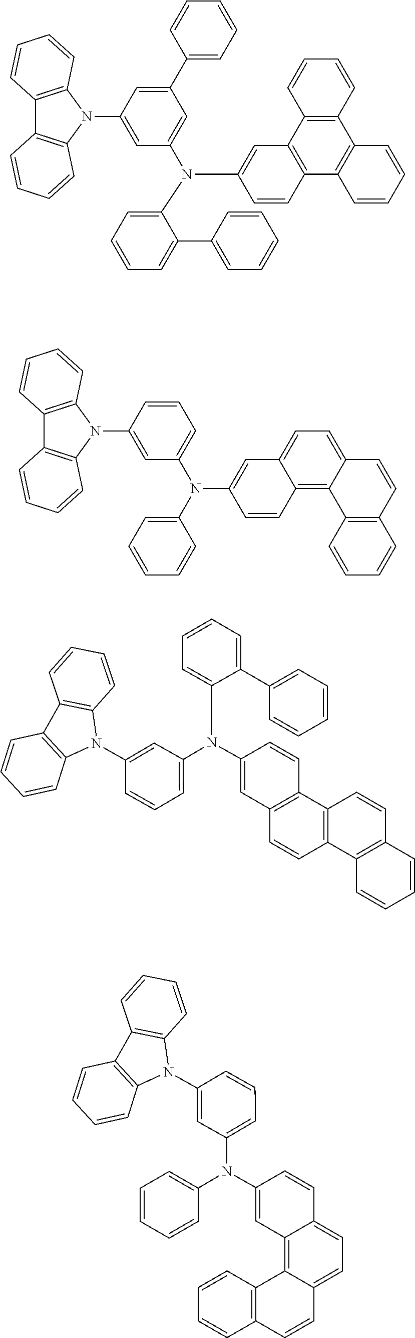 [SMILES: c1ccc(-c2cc(N(c3ccc4c5ccccc5c5ccccc5c4c3)c3ccccc3-c3ccccc3)cc(-n3c4ccccc4c4ccccc43)c2)cc1 ; c1ccc(N(c2cccc(-n3c4ccccc4c4ccccc43)c2)c2ccc3c(ccc4ccc5ccccc5c43)c2)cc1 ; c1ccc(-c2ccccc2N(c2cccc(-n3c4ccccc4c4ccccc43)c2)c2ccc3c(ccc4c5ccccc5ccc34)c2)cc1 ; c1ccc(N(c2cccc(-n3c4ccccc4c4ccccc43)c2)c2ccc3ccc4ccc5ccccc5c4c3c2)cc1] 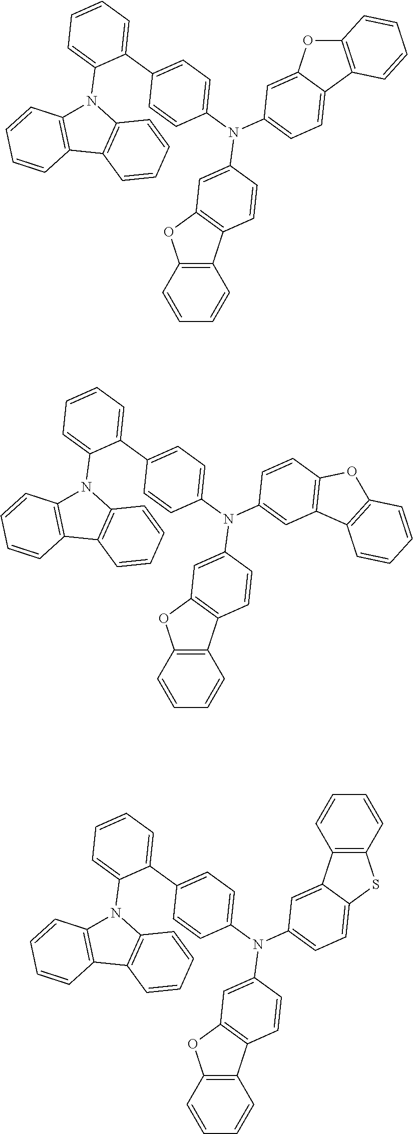 [SMILES: c1ccc(-n2c3ccccc3c3ccccc32)c(-c2ccc(N(c3ccc4c(c3)oc3ccccc34)c3ccc4c(c3)oc3ccccc34)cc2)c1 ; c1ccc(-n2c3ccccc3c3ccccc32)c(-c2ccc(N(c3ccc4c(c3)oc3ccccc34)c3ccc4oc5ccccc5c4c3)cc2)c1 ; c1ccc(-n2c3ccccc3c3ccccc32)c(-c2ccc(N(c3ccc4c(c3)oc3ccccc34)c3ccc4sc5ccccc5c4c3)cc2)c1] 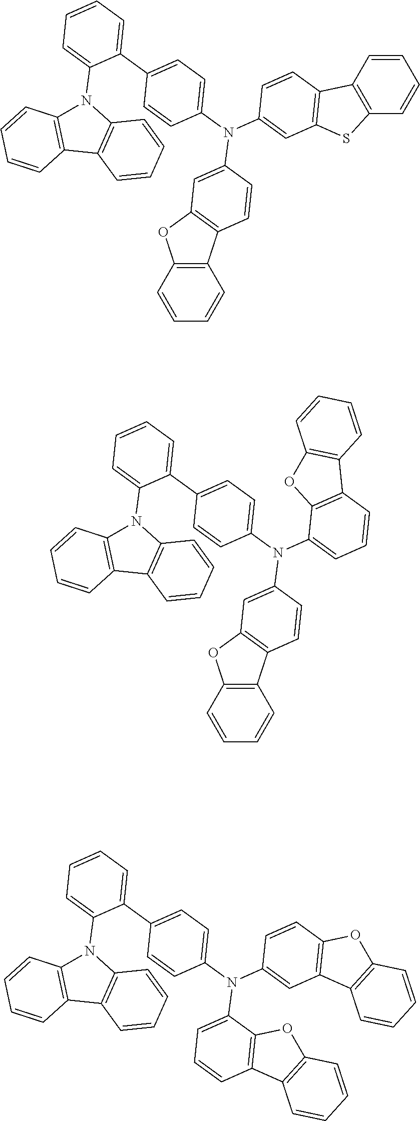 [SMILES: c1ccc(-n2c3ccccc3c3ccccc32)c(-c2ccc(N(c3ccc4c(c3)oc3ccccc34)c3ccc4c(c3)sc3ccccc34)cc2)c1 ; c1ccc(-n2c3ccccc3c3ccccc32)c(-c2ccc(N(c3ccc4c(c3)oc3ccccc34)c3cccc4c3oc3ccccc34)cc2)c1 ; c1ccc(-n2c3ccccc3c3ccccc32)c(-c2ccc(N(c3ccc4oc5ccccc5c4c3)c3cccc4c3oc3ccccc34)cc2)c1] 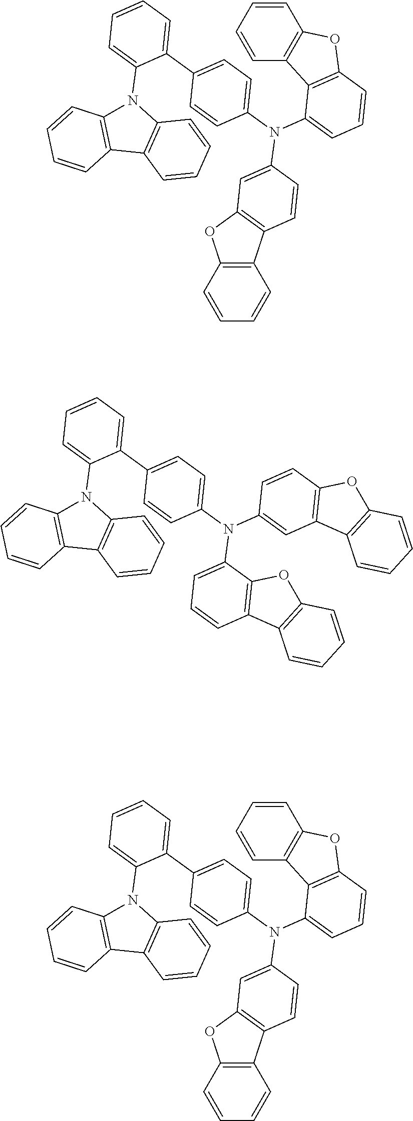 [SMILES: c1ccc(-n2c3ccccc3c3ccccc32)c(-c2ccc(N(c3ccc4c(c3)oc3ccccc34)c3cccc4oc5ccccc5c34)cc2)c1 ; c1ccc(-n2c3ccccc3c3ccccc32)c(-c2ccc(N(c3ccc4oc5ccccc5c4c3)c3cccc4c3oc3ccccc34)cc2)c1 ; c1ccc(-n2c3ccccc3c3ccccc32)c(-c2ccc(N(c3ccc4c(c3)oc3ccccc34)c3cccc4oc5ccccc5c34)cc2)c1] 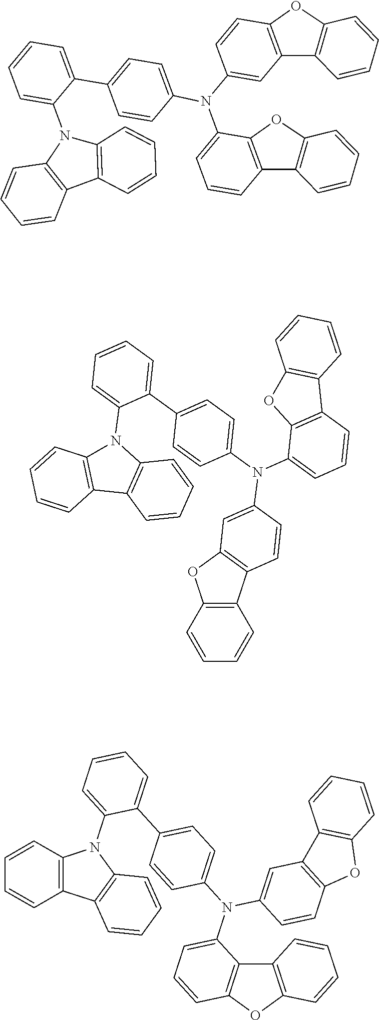 [SMILES: c1ccc(-n2c3ccccc3c3ccccc32)c(-c2ccc(N(c3ccc4oc5ccccc5c4c3)c3cccc4c3oc3ccccc34)cc2)c1 ; c1ccc(-n2c3ccccc3c3ccccc32)c(-c2ccc(N(c3ccc4c(c3)oc3ccccc34)c3cccc4c3oc3ccccc34)cc2)c1 ; c1ccc(-n2c3ccccc3c3ccccc32)c(-c2ccc(N(c3ccc4oc5ccccc5c4c3)c3cccc4oc5ccccc5c34)cc2)c1] 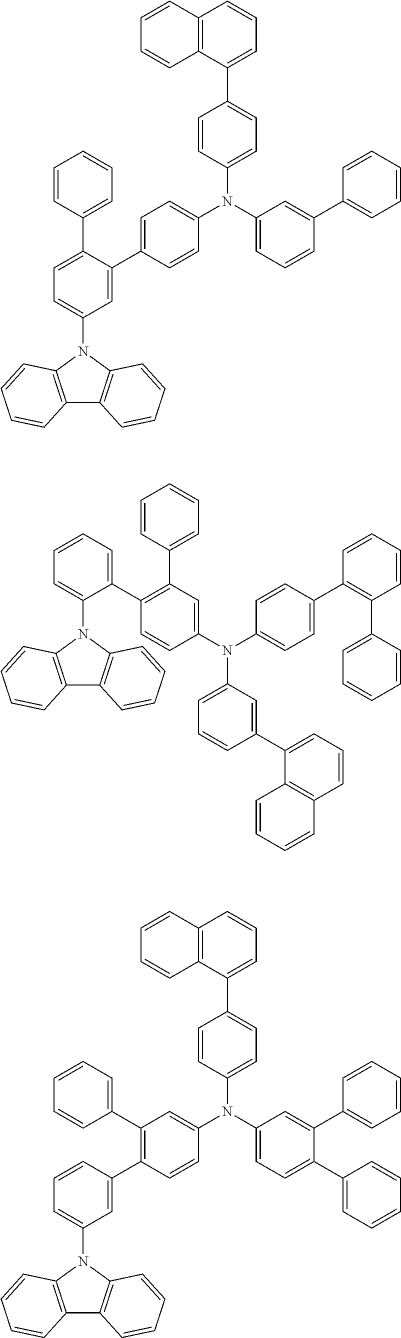 [SMILES: c1ccc(-c2cccc(N(c3ccc(-c4cc(-n5c6ccccc6c6ccccc65)ccc4-c4ccccc4)cc3)c3ccc(-c4cccc5ccccc45)cc3)c2)cc1 ; c1ccc(-c2ccccc2-c2ccc(N(c3cccc(-c4cccc5ccccc45)c3)c3ccc(-c4ccccc4-n4c5ccccc5c5ccccc54)c(-c4ccccc4)c3)cc2)cc1 ; c1ccc(-c2ccc(N(c3ccc(-c4cccc5ccccc45)cc3)c3ccc(-c4cccc(-n5c6ccccc6c6ccccc65)c4)c(-c4ccccc4)c3)cc2-c2ccccc2)cc1] 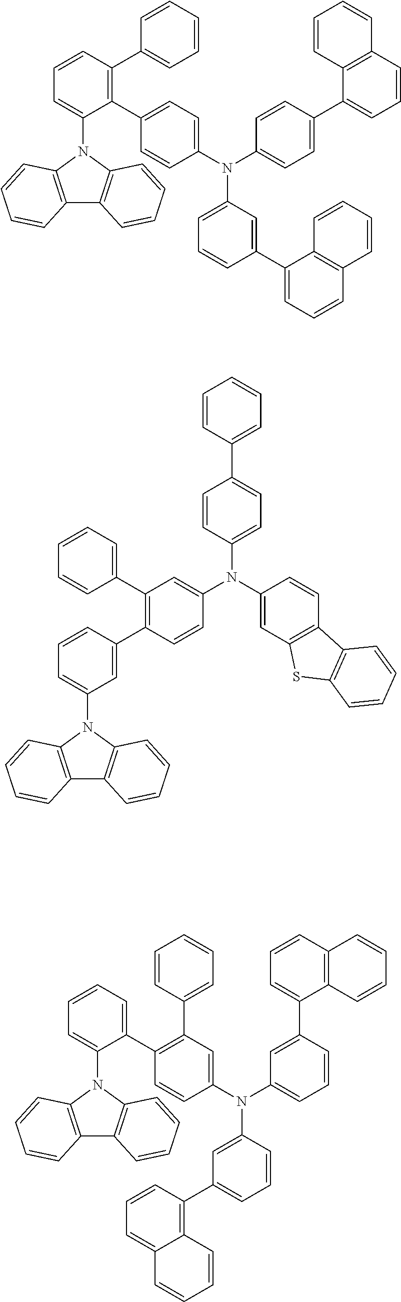 [SMILES: c1ccc(-c2cccc(-n3c4ccccc4c4ccccc43)c2-c2ccc(N(c3ccc(-c4cccc5ccccc45)cc3)c3cccc(-c4cccc5ccccc45)c3)cc2)cc1 ; c1ccc(-c2ccc(N(c3ccc(-c4cccc(-n5c6ccccc6c6ccccc65)c4)c(-c4ccccc4)c3)c3ccc4c(c3)sc3ccccc34)cc2)cc1 ; c1ccc(-c2cc(N(c3cccc(-c4cccc5ccccc45)c3)c3cccc(-c4cccc5ccccc45)c3)ccc2-c2ccccc2-n2c3ccccc3c3ccccc32)cc1] 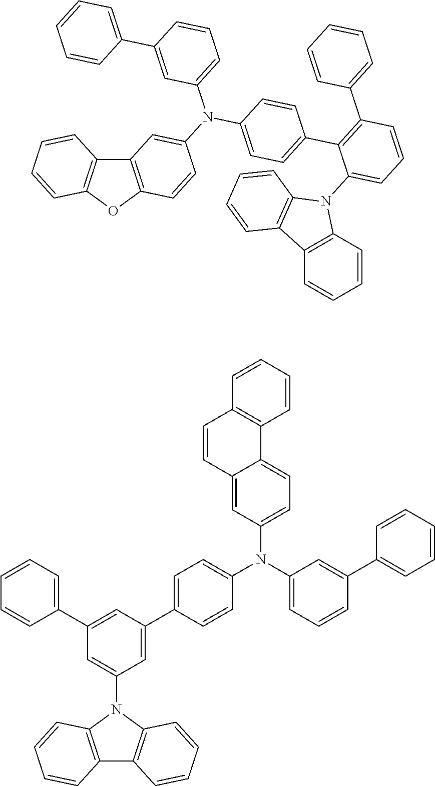 [SMILES: c1ccc(-c2cccc(N(c3ccc(-c4c(-c5ccccc5)cccc4-n4c5ccccc5c5ccccc54)cc3)c3ccc4oc5ccccc5c4c3)c2)cc1 ; c1ccc(-c2cccc(N(c3ccc(-c4cc(-c5ccccc5)cc(-n5c6ccccc6c6ccccc65)c4)cc3)c3ccc4c(ccc5ccccc54)c3)c2)cc1] 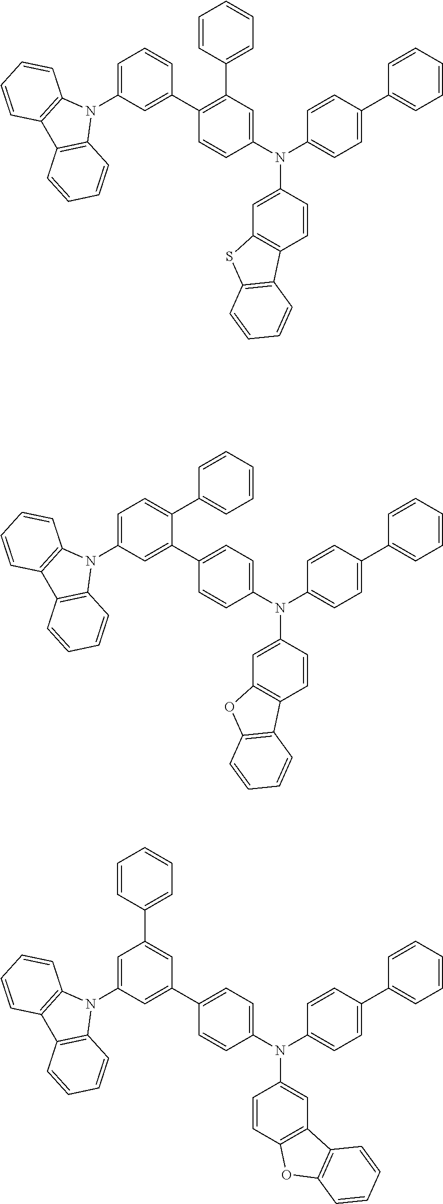 [SMILES: c1ccc(-c2ccc(N(c3ccc(-c4cccc(-n5c6ccccc6c6ccccc65)c4)c(-c4ccccc4)c3)c3ccc4c(c3)sc3ccccc34)cc2)cc1 ; c1ccc(-c2ccc(N(c3ccc(-c4cc(-n5c6ccccc6c6ccccc65)ccc4-c4ccccc4)cc3)c3ccc4c(c3)oc3ccccc34)cc2)cc1 ; c1ccc(-c2ccc(N(c3ccc(-c4cc(-c5ccccc5)cc(-n5c6ccccc6c6ccccc65)c4)cc3)c3ccc4oc5ccccc5c4c3)cc2)cc1] 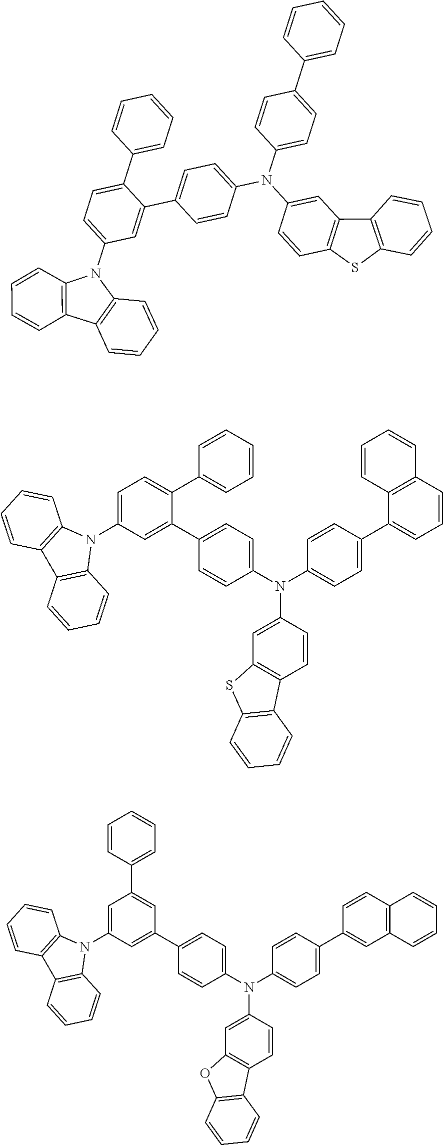 [SMILES: c1ccc(-c2ccc(N(c3ccc(-c4cc(-n5c6ccccc6c6ccccc65)ccc4-c4ccccc4)cc3)c3ccc4sc5ccccc5c4c3)cc2)cc1 ; c1ccc(-c2ccc(-n3c4ccccc4c4ccccc43)cc2-c2ccc(N(c3ccc(-c4cccc5ccccc45)cc3)c3ccc4c(c3)sc3ccccc34)cc2)cc1 ; c1ccc(-c2cc(-c3ccc(N(c4ccc(-c5ccc6ccccc6c5)cc4)c4ccc5c(c4)oc4ccccc45)cc3)cc(-n3c4ccccc4c4ccccc43)c2)cc1] 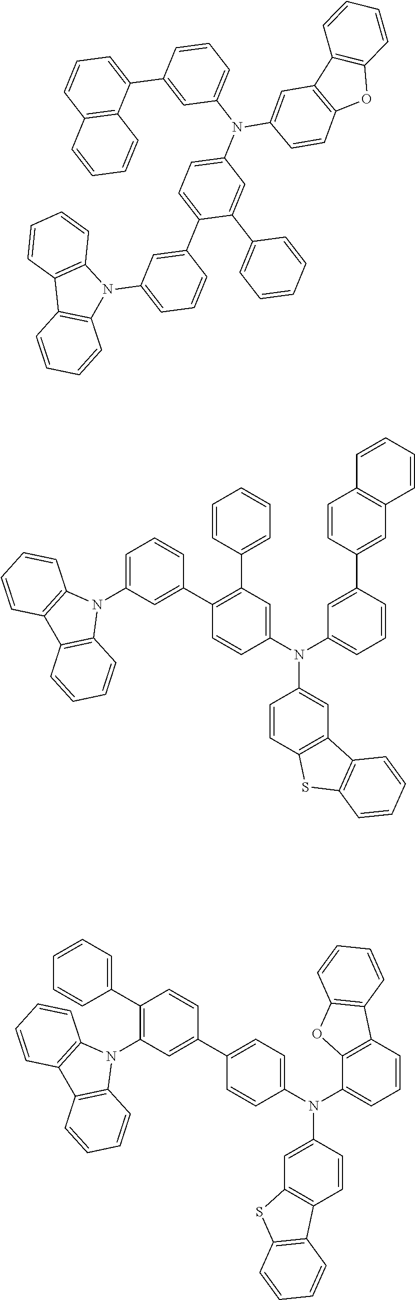 [SMILES: c1ccc(-c2cc(N(c3cccc(-c4cccc5ccccc45)c3)c3ccc4oc5ccccc5c4c3)ccc2-c2cccc(-n3c4ccccc4c4ccccc43)c2)cc1 ; c1ccc(-c2cc(N(c3cccc(-c4ccc5ccccc5c4)c3)c3ccc4sc5ccccc5c4c3)ccc2-c2cccc(-n3c4ccccc4c4ccccc43)c2)cc1 ; c1ccc(-c2ccc(-c3ccc(N(c4ccc5c(c4)sc4ccccc45)c4cccc5c4oc4ccccc45)cc3)cc2-n2c3ccccc3c3ccccc32)cc1] 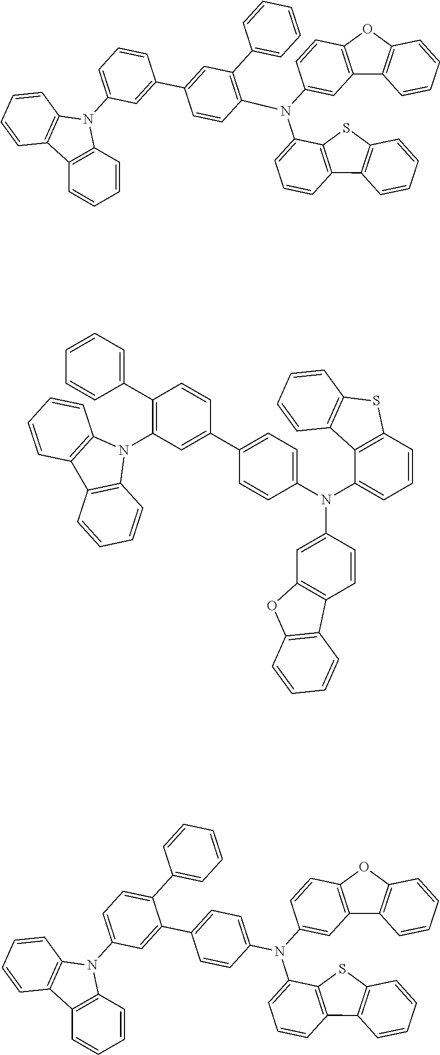 [SMILES: c1ccc(-c2cc(-c3cccc(-n4c5ccccc5c5ccccc54)c3)ccc2N(c2ccc3oc4ccccc4c3c2)c2cccc3c2sc2ccccc23)cc1 ; c1ccc(-c2ccc(-c3ccc(N(c4ccc5c(c4)oc4ccccc45)c4cccc5sc6ccccc6c45)cc3)cc2-n2c3ccccc3c3ccccc32)cc1 ; c1ccc(-c2ccc(-n3c4ccccc4c4ccccc43)cc2-c2ccc(N(c3ccc4oc5ccccc5c4c3)c3cccc4c3sc3ccccc34)cc2)cc1] 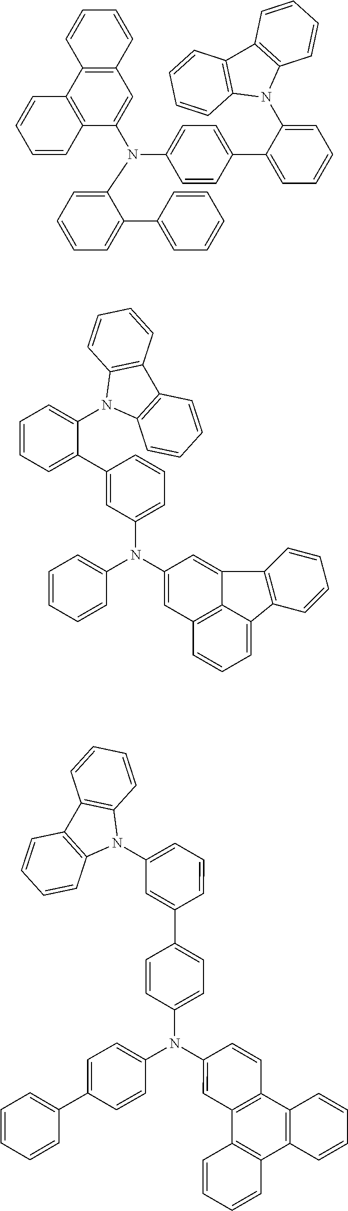 [SMILES: c1ccc(-c2ccccc2N(c2ccc(-c3ccccc3-n3c4ccccc4c4ccccc43)cc2)c2cc3ccccc3c3ccccc23)cc1 ; c1ccc(N(c2cccc(-c3ccccc3-n3c4ccccc4c4ccccc43)c2)c2cc3c4c(cccc4c2)-c2ccccc2-3)cc1 ; c1ccc(-c2ccc(N(c3ccc(-c4cccc(-n5c6ccccc6c6ccccc65)c4)cc3)c3ccc4c5ccccc5c5ccccc5c4c3)cc2)cc1] 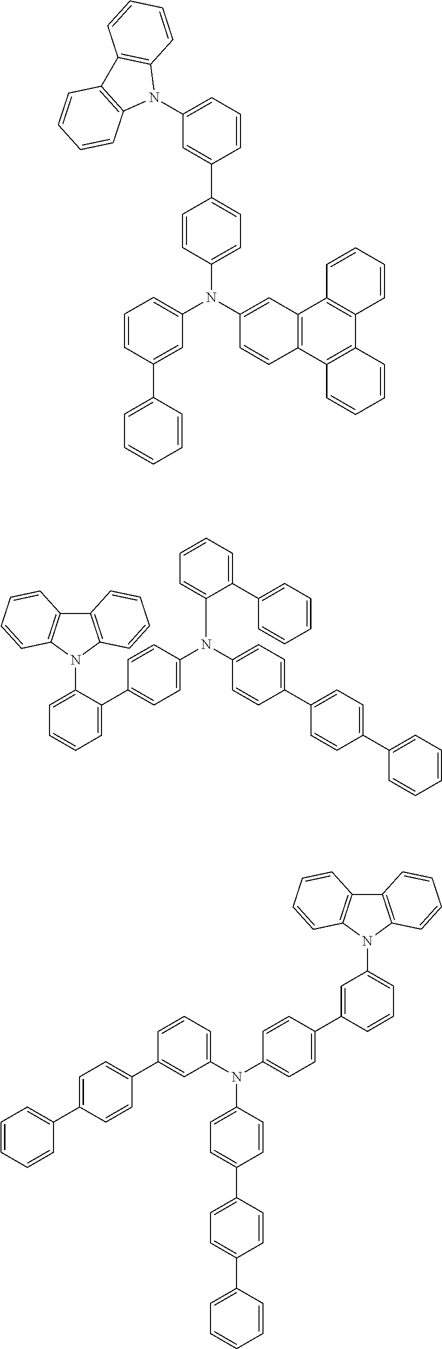 [SMILES: c1ccc(-c2cccc(N(c3ccc(-c4cccc(-n5c6ccccc6c6ccccc65)c4)cc3)c3ccc4c5ccccc5c5ccccc5c4c3)c2)cc1 ; c1ccc(-c2ccc(-c3ccc(N(c4ccc(-c5ccccc5-n5c6ccccc6c6ccccc65)cc4)c4ccccc4-c4ccccc4)cc3)cc2)cc1 ; c1ccc(-c2ccc(-c3ccc(N(c4ccc(-c5cccc(-n6c7ccccc7c7ccccc76)c5)cc4)c4cccc(-c5ccc(-c6ccccc6)cc5)c4)cc3)cc2)cc1] 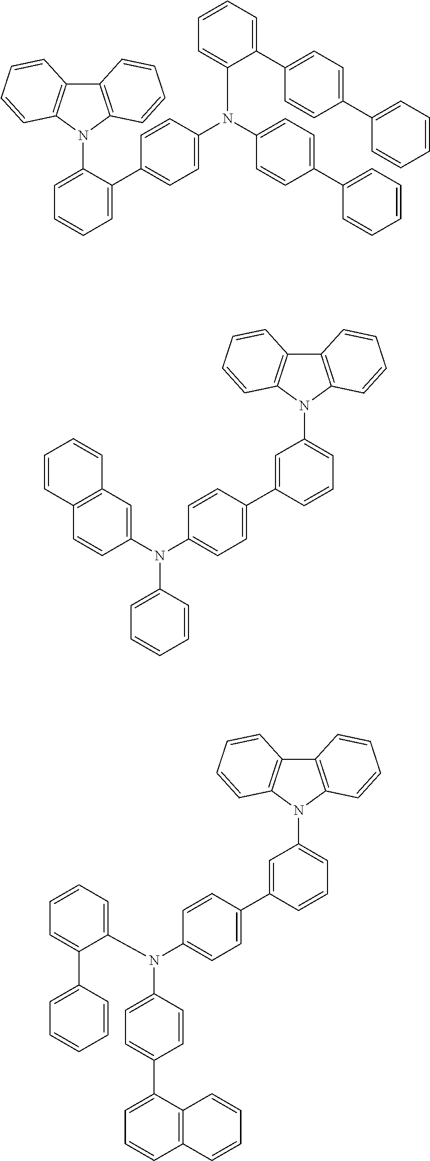 [SMILES: c1ccc(-c2ccc(-c3ccccc3N(c3ccc(-c4ccccc4)cc3)c3ccc(-c4ccccc4-n4c5ccccc5c5ccccc54)cc3)cc2)cc1 ; c1ccc(N(c2ccc(-c3cccc(-n4c5ccccc5c5ccccc54)c3)cc2)c2ccc3ccccc3c2)cc1 ; c1ccc(-c2ccccc2N(c2ccc(-c3cccc(-n4c5ccccc5c5ccccc54)c3)cc2)c2ccc(-c3cccc4ccccc34)cc2)cc1] 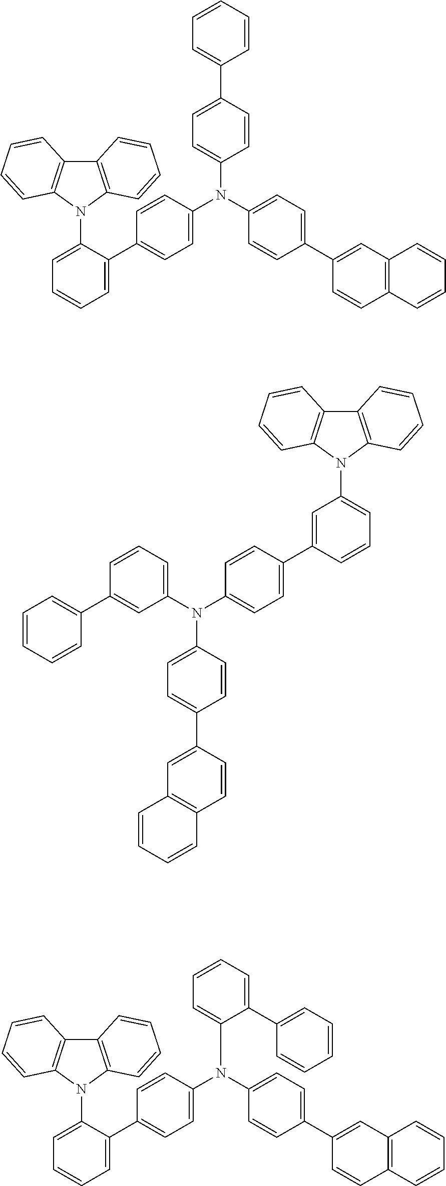 [SMILES: c1ccc(-c2ccc(N(c3ccc(-c4ccc5ccccc5c4)cc3)c3ccc(-c4ccccc4-n4c5ccccc5c5ccccc54)cc3)cc2)cc1 ; c1ccc(-c2cccc(N(c3ccc(-c4cccc(-n5c6ccccc6c6ccccc65)c4)cc3)c3ccc(-c4ccc5ccccc5c4)cc3)c2)cc1 ; c1ccc(-c2ccccc2N(c2ccc(-c3ccc4ccccc4c3)cc2)c2ccc(-c3ccccc3-n3c4ccccc4c4ccccc43)cc2)cc1] 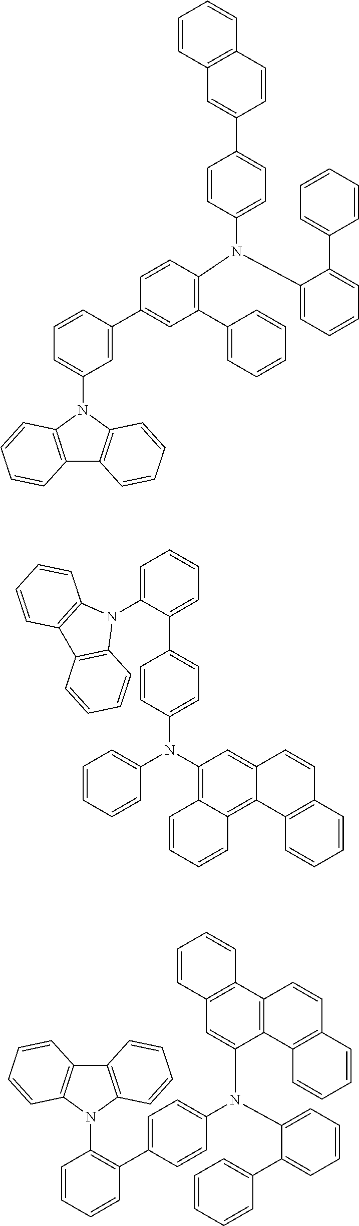 [SMILES: c1ccc(-c2ccccc2N(c2ccc(-c3ccc4ccccc4c3)cc2)c2ccc(-c3cccc(-n4c5ccccc5c5ccccc54)c3)cc2-c2ccccc2)cc1 ; c1ccc(N(c2ccc(-c3ccccc3-n3c4ccccc4c4ccccc43)cc2)c2cc3ccc4ccccc4c3c3ccccc23)cc1 ; c1ccc(-c2ccccc2N(c2ccc(-c3ccccc3-n3c4ccccc4c4ccccc43)cc2)c2cc3ccccc3c3ccc4ccccc4c23)cc1] 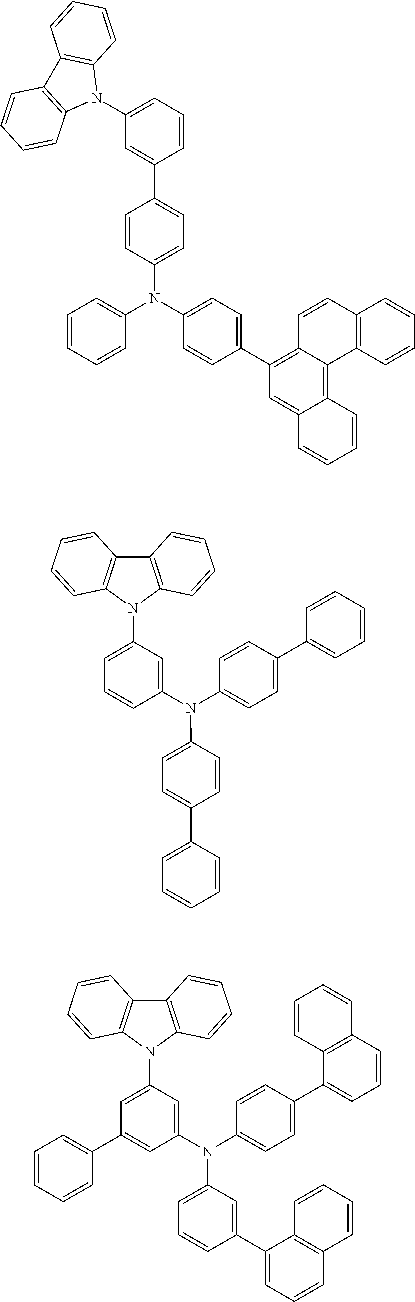 [SMILES: c1ccc(N(c2ccc(-c3cccc(-n4c5ccccc5c5ccccc54)c3)cc2)c2ccc(-c3cc4ccccc4c4c3ccc3ccccc34)cc2)cc1 ; c1ccc(-c2ccc(N(c3ccc(-c4ccccc4)cc3)c3cccc(-n4c5ccccc5c5ccccc54)c3)cc2)cc1 ; c1ccc(-c2cc(N(c3ccc(-c4cccc5ccccc45)cc3)c3cccc(-c4cccc5ccccc45)c3)cc(-n3c4ccccc4c4ccccc43)c2)cc1] 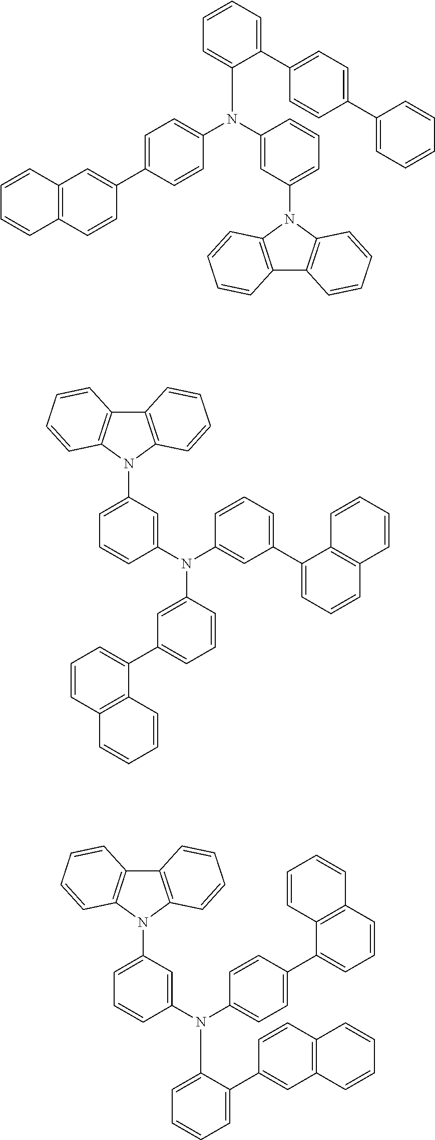 [SMILES: c1ccc(-c2ccc(-c3ccccc3N(c3ccc(-c4ccc5ccccc5c4)cc3)c3cccc(-n4c5ccccc5c5ccccc54)c3)cc2)cc1 ; c1cc(-c2cccc3ccccc23)cc(N(c2cccc(-c3cccc4ccccc34)c2)c2cccc(-n3c4ccccc4c4ccccc43)c2)c1 ; c1cc(N(c2ccc(-c3cccc4ccccc34)cc2)c2ccccc2-c2ccc3ccccc3c2)cc(-n2c3ccccc3c3ccccc32)c1] 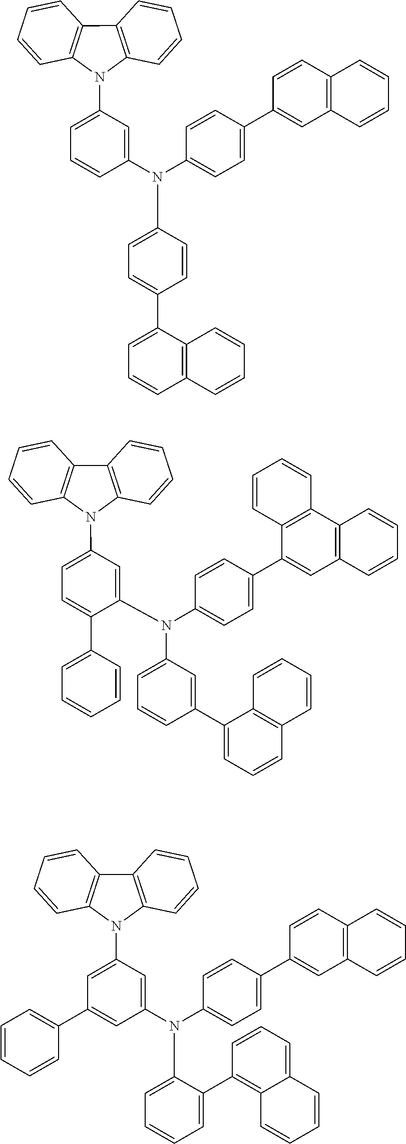 [SMILES: c1cc(N(c2ccc(-c3ccc4ccccc4c3)cc2)c2ccc(-c3cccc4ccccc34)cc2)cc(-n2c3ccccc3c3ccccc32)c1 ; c1ccc(-c2ccc(-n3c4ccccc4c4ccccc43)cc2N(c2ccc(-c3cc4ccccc4c4ccccc34)cc2)c2cccc(-c3cccc4ccccc34)c2)cc1 ; c1ccc(-c2cc(N(c3ccc(-c4ccc5ccccc5c4)cc3)c3ccccc3-c3cccc4ccccc34)cc(-n3c4ccccc4c4ccccc43)c2)cc1] 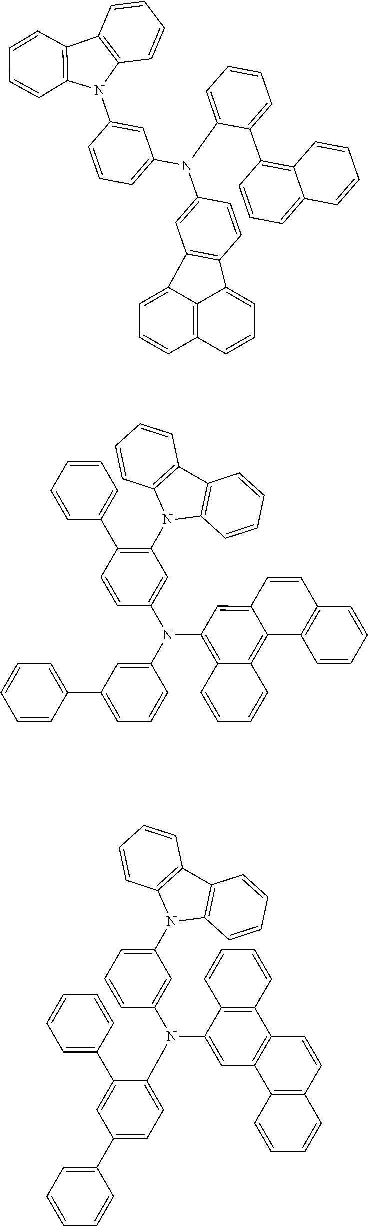 [SMILES: c1cc(N(c2ccc3c(c2)-c2cccc4cccc-3c24)c2ccccc2-c2cccc3ccccc23)cc(-n2c3ccccc3c3ccccc32)c1 ; c1ccc(-c2cccc(N(c3ccc(-c4ccccc4)c(-n4c5ccccc5c5ccccc54)c3)c3cc4ccc5ccccc5c4c4ccccc34)c2)cc1 ; c1ccc(-c2ccc(N(c3cccc(-n4c5ccccc5c5ccccc54)c3)c3cc4c5ccccc5ccc4c4ccccc34)c(-c3ccccc3)c2)cc1] 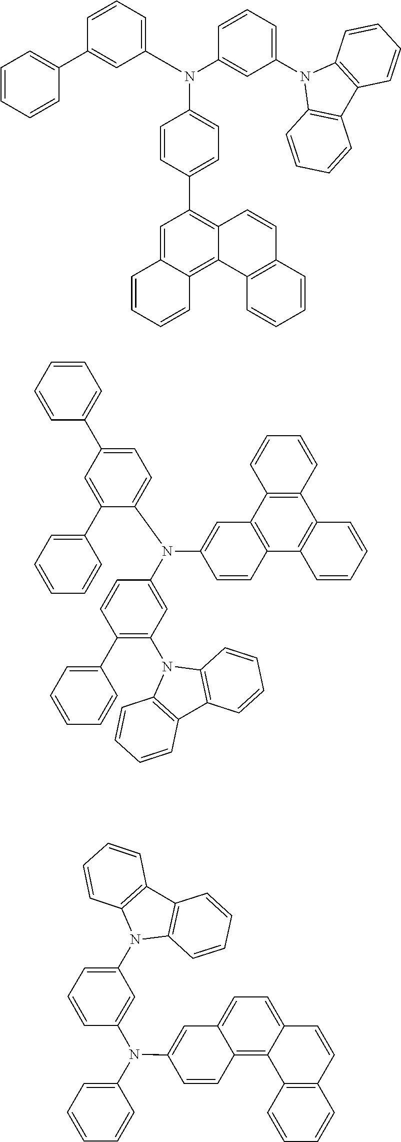 [SMILES: c1ccc(-c2cccc(N(c3ccc(-c4cc5ccccc5c5c4ccc4ccccc45)cc3)c3cccc(-n4c5ccccc5c5ccccc54)c3)c2)cc1 ; c1ccc(-c2ccc(N(c3ccc(-c4ccccc4)c(-n4c5ccccc5c5ccccc54)c3)c3ccc4c5ccccc5c5ccccc5c4c3)c(-c3ccccc3)c2)cc1 ; c1ccc(N(c2cccc(-n3c4ccccc4c4ccccc43)c2)c2ccc3c(ccc4ccc5ccccc5c43)c2)cc1] 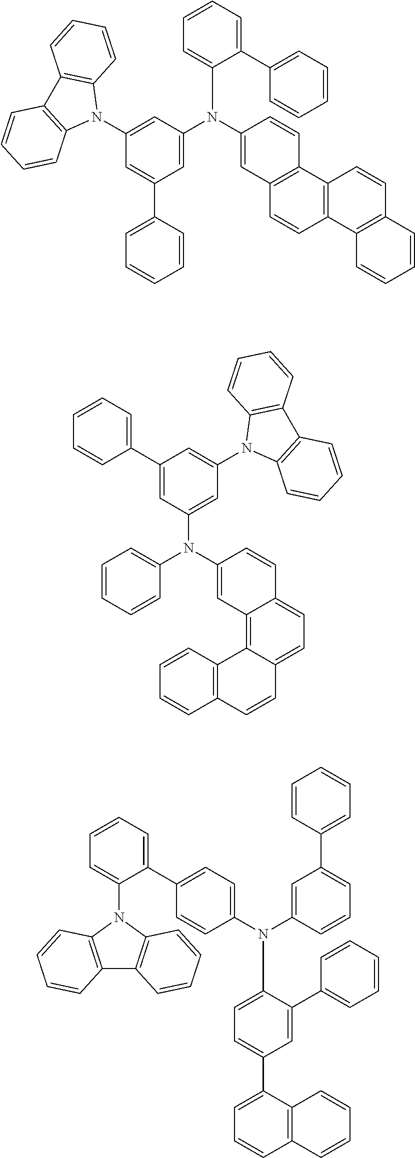 [SMILES: c1ccc(-c2cc(N(c3ccc4c(ccc5c6ccccc6ccc45)c3)c3ccccc3-c3ccccc3)cc(-n3c4ccccc4c4ccccc43)c2)cc1 ; c1ccc(-c2cc(N(c3ccccc3)c3ccc4ccc5ccc6ccccc6c5c4c3)cc(-n3c4ccccc4c4ccccc43)c2)cc1 ; c1ccc(-c2cccc(N(c3ccc(-c4ccccc4-n4c5ccccc5c5ccccc54)cc3)c3ccc(-c4cccc5ccccc45)cc3-c3ccccc3)c2)cc1] 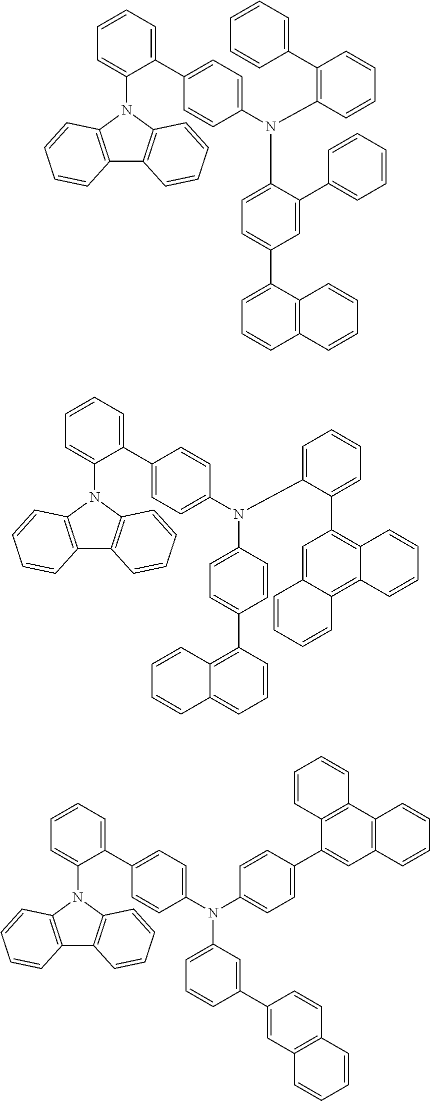 [SMILES: c1ccc(-c2ccccc2N(c2ccc(-c3ccccc3-n3c4ccccc4c4ccccc43)cc2)c2ccc(-c3cccc4ccccc34)cc2-c2ccccc2)cc1 ; c1ccc(N(c2ccc(-c3ccccc3-n3c4ccccc4c4ccccc43)cc2)c2ccc(-c3cccc4ccccc34)cc2)c(-c2cc3ccccc3c3ccccc23)c1 ; c1cc(-c2ccc3ccccc3c2)cc(N(c2ccc(-c3ccccc3-n3c4ccccc4c4ccccc43)cc2)c2ccc(-c3cc4ccccc4c4ccccc34)cc2)c1] 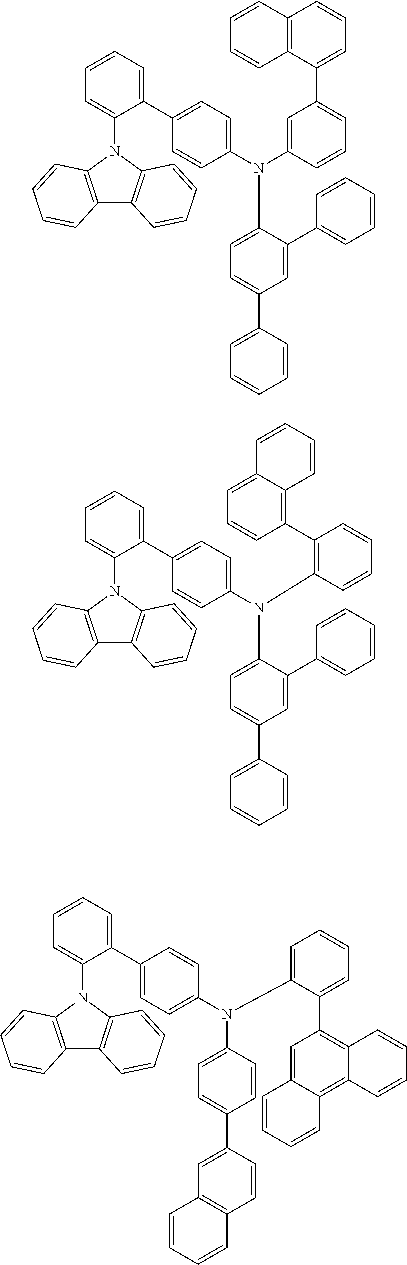 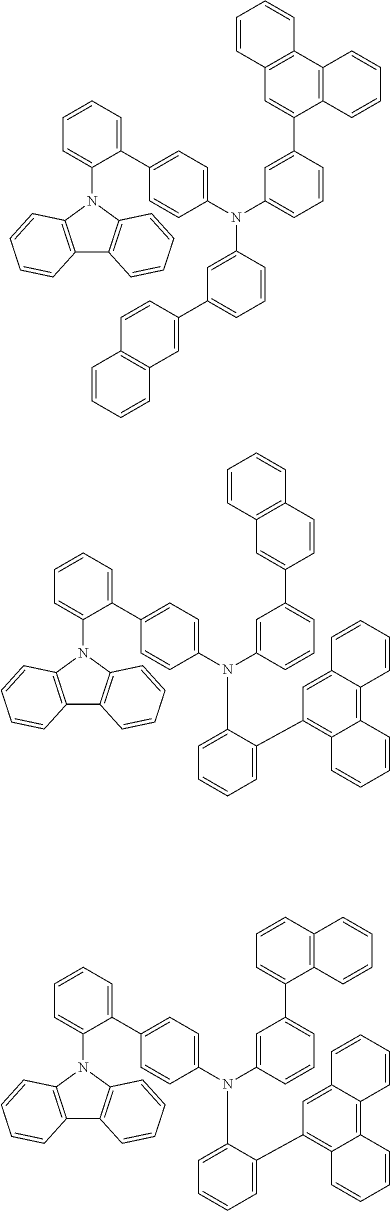 [SMILES: c1cc(-c2ccc3ccccc3c2)cc(N(c2ccc(-c3ccccc3-n3c4ccccc4c4ccccc43)cc2)c2cccc(-c3cc4ccccc4c4ccccc34)c2)c1 ; c1cc(-c2ccc3ccccc3c2)cc(N(c2ccc(-c3ccccc3-n3c4ccccc4c4ccccc43)cc2)c2ccccc2-c2cc3ccccc3c3ccccc23)c1 ; c1cc(-c2cccc3ccccc23)cc(N(c2ccc(-c3ccccc3-n3c4ccccc4c4ccccc43)cc2)c2ccccc2-c2cc3ccccc3c3ccccc23)c1] 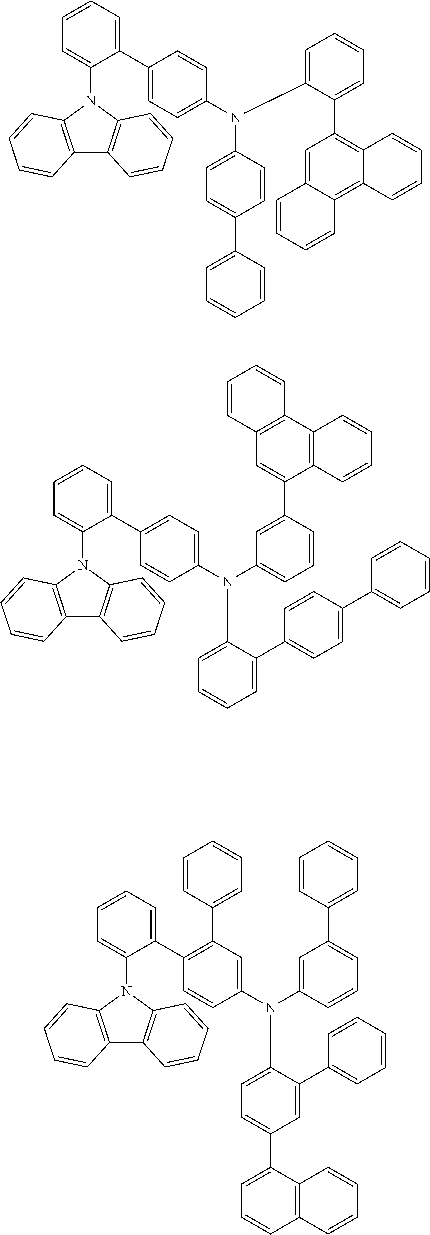 [SMILES: c1ccc(-c2ccc(N(c3ccc(-c4ccccc4-n4c5ccccc5c5ccccc54)cc3)c3ccccc3-c3cc4ccccc4c4ccccc34)cc2)cc1 ; c1ccc(-c2ccc(-c3ccccc3N(c3ccc(-c4ccccc4-n4c5ccccc5c5ccccc54)cc3)c3cccc(-c4cc5ccccc5c5ccccc45)c3)cc2)cc1 ; c1ccc(-c2cccc(N(c3ccc(-c4ccccc4-n4c5ccccc5c5ccccc54)c(-c4ccccc4)c3)c3ccc(-c4cccc5ccccc45)cc3-c3ccccc3)c2)cc1] 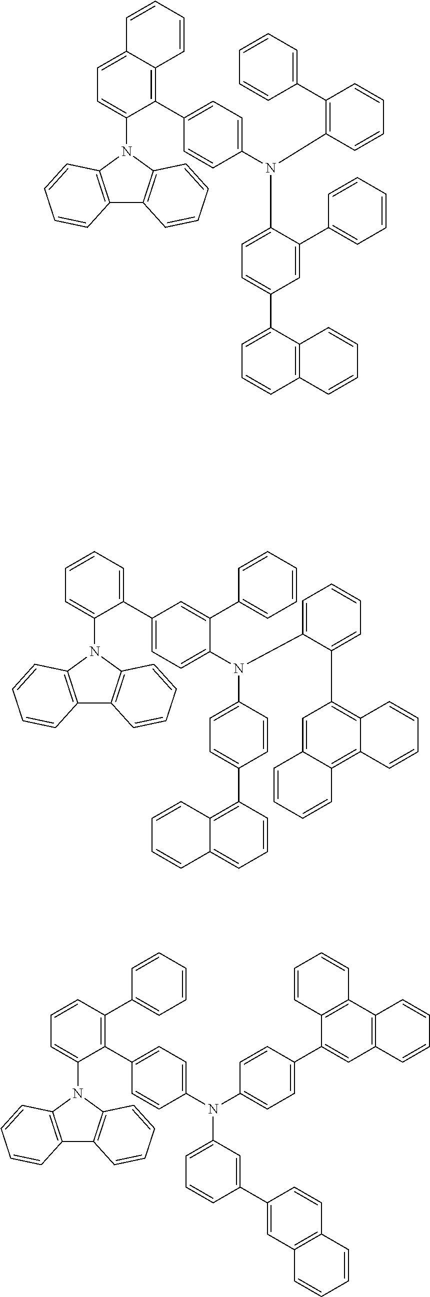 [SMILES: c1ccc(-c2ccccc2N(c2ccc(-c3c(-n4c5ccccc5c5ccccc54)ccc4ccccc34)cc2)c2ccc(-c3cccc4ccccc34)cc2-c2ccccc2)cc1 ; c1ccc(-c2cc(-c3ccccc3-n3c4ccccc4c4ccccc43)ccc2N(c2ccc(-c3cccc4ccccc34)cc2)c2ccccc2-c2cc3ccccc3c3ccccc23)cc1 ; c1ccc(-c2cccc(-n3c4ccccc4c4ccccc43)c2-c2ccc(N(c3ccc(-c4cc5ccccc5c5ccccc45)cc3)c3cccc(-c4ccc5ccccc5c4)c3)cc2)cc1] 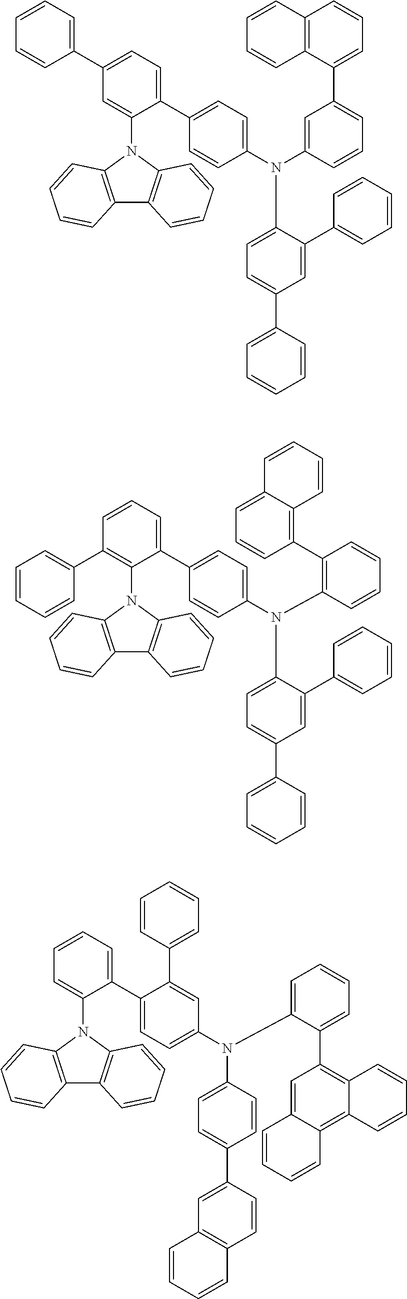 [SMILES: c1ccc(-c2ccc(N(c3ccc(-c4ccc(-c5ccccc5)cc4-n4c5ccccc5c5ccccc54)cc3)c3cccc(-c4cccc5ccccc45)c3)c(-c3ccccc3)c2)cc1 ; c1ccc(-c2ccc(N(c3ccc(-c4cccc(-c5ccccc5)c4-n4c5ccccc5c5ccccc54)cc3)c3ccccc3-c3cccc4ccccc34)c(-c3ccccc3)c2)cc1 ; c1ccc(-c2cc(N(c3ccc(-c4ccc5ccccc5c4)cc3)c3ccccc3-c3cc4ccccc4c4ccccc34)ccc2-c2ccccc2-n2c3ccccc3c3ccccc32)cc1] 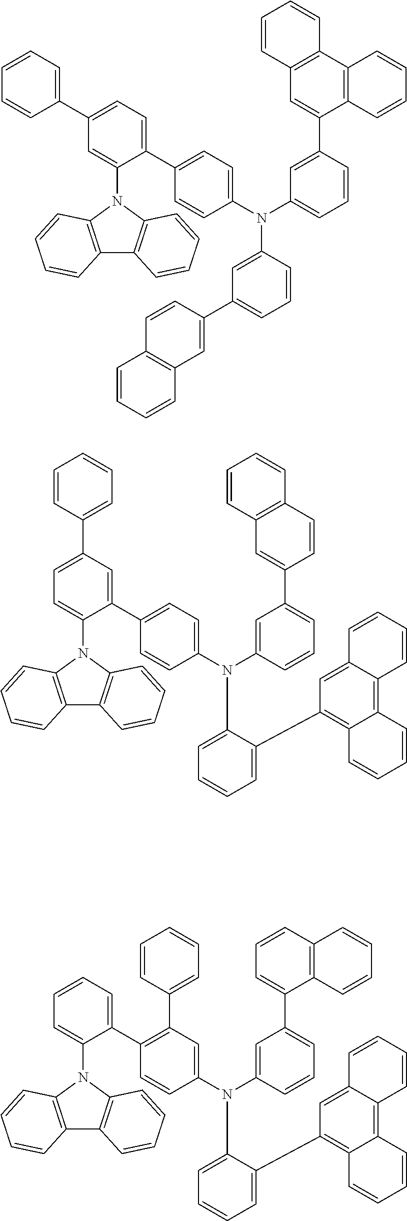 [SMILES: c1ccc(-c2ccc(-c3ccc(N(c4cccc(-c5ccc6ccccc6c5)c4)c4cccc(-c5cc6ccccc6c6ccccc56)c4)cc3)c(-n3c4ccccc4c4ccccc43)c2)cc1 ; c1ccc(-c2ccc(-n3c4ccccc4c4ccccc43)c(-c3ccc(N(c4cccc(-c5ccc6ccccc6c5)c4)c4ccccc4-c4cc5ccccc5c5ccccc45)cc3)c2)cc1 ; c1ccc(-c2cc(N(c3cccc(-c4cccc5ccccc45)c3)c3ccccc3-c3cc4ccccc4c4ccccc34)ccc2-c2ccccc2-n2c3ccccc3c3ccccc32)cc1] 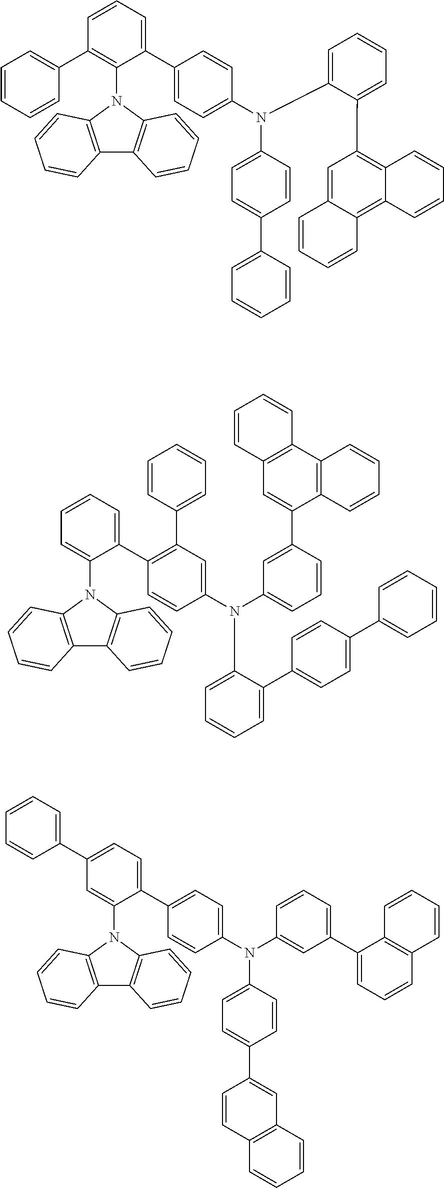 [SMILES: c1ccc(-c2ccc(N(c3ccc(-c4cccc(-c5ccccc5)c4-n4c5ccccc5c5ccccc54)cc3)c3ccccc3-c3cc4ccccc4c4ccccc34)cc2)cc1 ; c1ccc(-c2ccc(-c3ccccc3N(c3cccc(-c4cc5ccccc5c5ccccc45)c3)c3ccc(-c4ccccc4-n4c5ccccc5c5ccccc54)c(-c4ccccc4)c3)cc2)cc1 ; c1ccc(-c2ccc(-c3ccc(N(c4ccc(-c5ccc6ccccc6c5)cc4)c4cccc(-c5cccc6ccccc56)c4)cc3)c(-n3c4ccccc4c4ccccc43)c2)cc1] 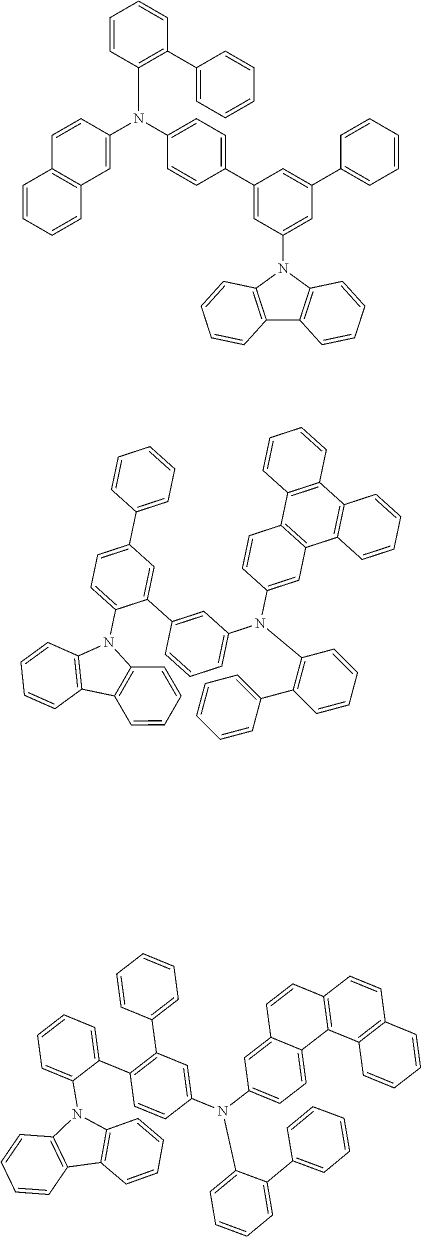 [SMILES: c1ccc(-c2cc(-c3ccc(N(c4ccc5ccccc5c4)c4ccccc4-c4ccccc4)cc3)cc(-n3c4ccccc4c4ccccc43)c2)cc1 ; c1ccc(-c2ccc(-n3c4ccccc4c4ccccc43)c(-c3cccc(N(c4ccc5c6ccccc6c6ccccc6c5c4)c4ccccc4-c4ccccc4)c3)c2)cc1 ; c1ccc(-c2cc(N(c3ccc4c(ccc5ccc6ccccc6c54)c3)c3ccccc3-c3ccccc3)ccc2-c2ccccc2-n2c3ccccc3c3ccccc32)cc1] 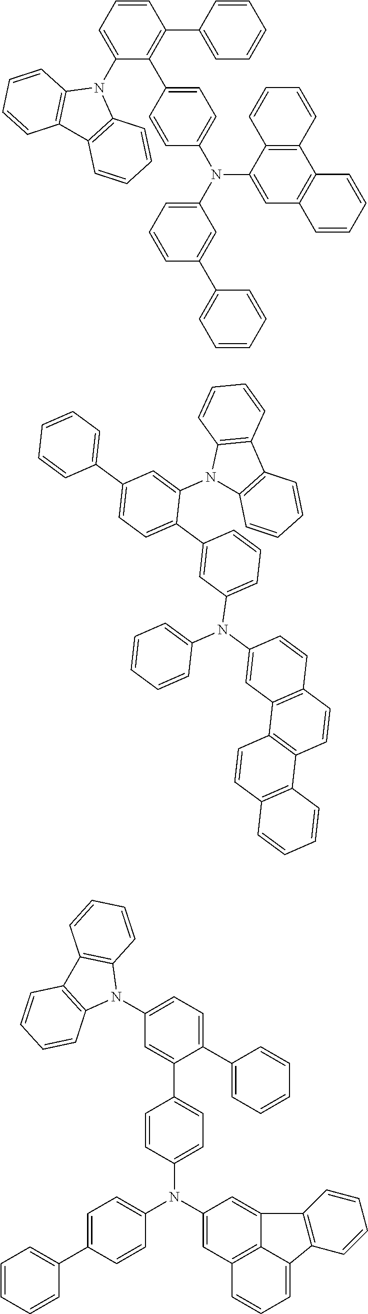 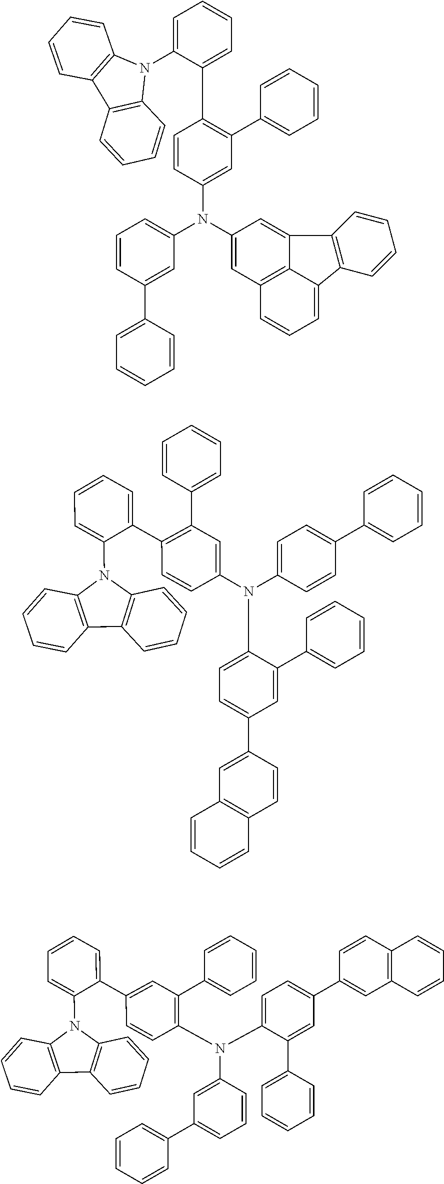 [SMILES: c1ccc(-c2cccc(N(c3ccc(-c4ccccc4-n4c5ccccc5c5ccccc54)c(-c4ccccc4)c3)c3cc4c5c(cccc5c3)-c3ccccc3-4)c2)cc1 ; c1ccc(-c2ccc(N(c3ccc(-c4ccccc4-n4c5ccccc5c5ccccc54)c(-c4ccccc4)c3)c3ccc(-c4ccc5ccccc5c4)cc3-c3ccccc3)cc2)cc1 ; c1ccc(-c2cccc(N(c3ccc(-c4ccc5ccccc5c4)cc3-c3ccccc3)c3ccc(-c4ccccc4-n4c5ccccc5c5ccccc54)cc3-c3ccccc3)c2)cc1] 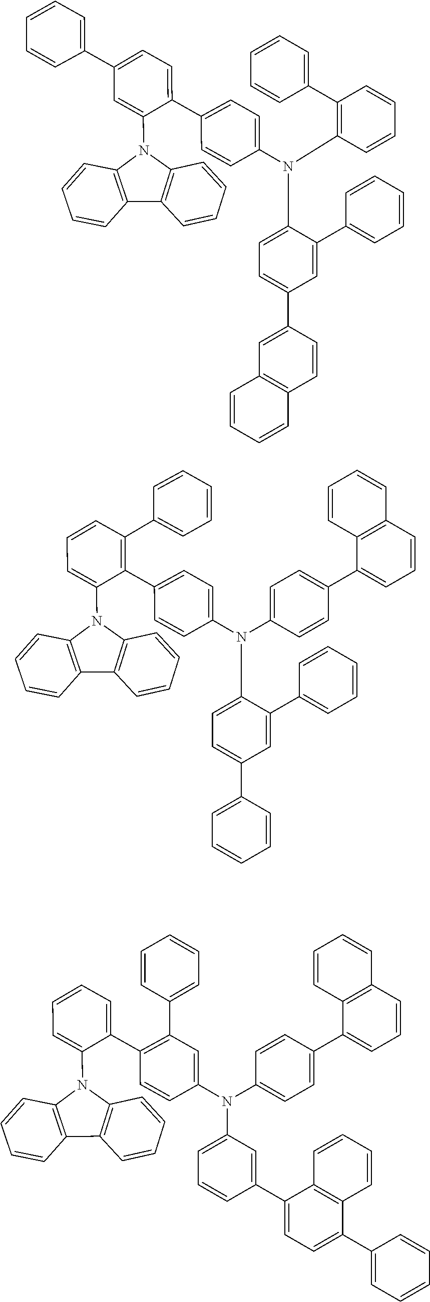 [SMILES: c1ccc(-c2ccc(-c3ccc(N(c4ccccc4-c4ccccc4)c4ccc(-c5ccc6ccccc6c5)cc4-c4ccccc4)cc3)c(-n3c4ccccc4c4ccccc43)c2)cc1 ; c1ccc(-c2ccc(N(c3ccc(-c4c(-c5ccccc5)cccc4-n4c5ccccc5c5ccccc54)cc3)c3ccc(-c4cccc5ccccc45)cc3)c(-c3ccccc3)c2)cc1 ; c1ccc(-c2cc(N(c3ccc(-c4cccc5ccccc45)cc3)c3cccc(-c4ccc(-c5ccccc5)c5ccccc45)c3)ccc2-c2ccccc2-n2c3ccccc3c3ccccc32)cc1] 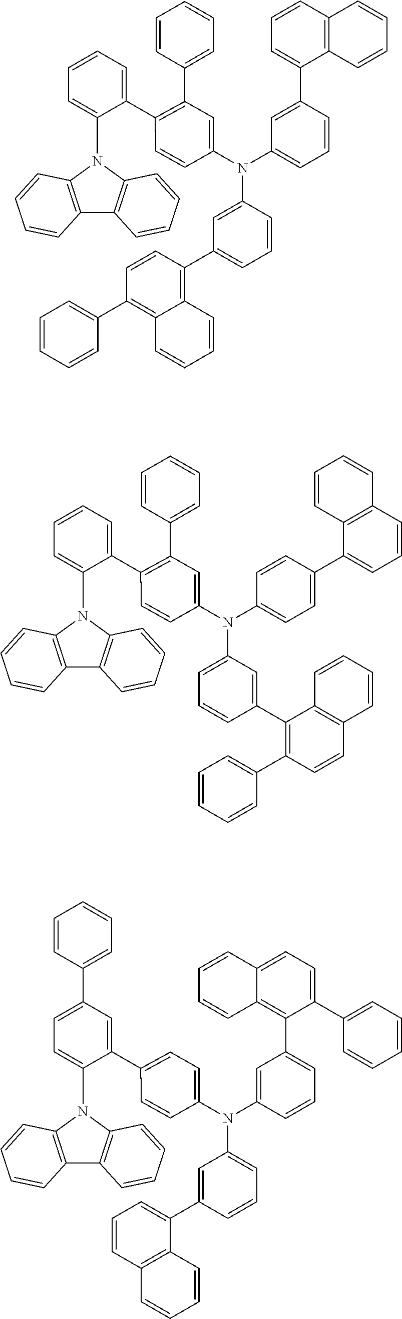 [SMILES: c1ccc(-c2cc(N(c3cccc(-c4cccc5ccccc45)c3)c3cccc(-c4ccc(-c5ccccc5)c5ccccc45)c3)ccc2-c2ccccc2-n2c3ccccc3c3ccccc32)cc1 ; c1ccc(-c2cc(N(c3ccc(-c4cccc5ccccc45)cc3)c3cccc(-c4c(-c5ccccc5)ccc5ccccc45)c3)ccc2-c2ccccc2-n2c3ccccc3c3ccccc32)cc1 ; c1ccc(-c2ccc(-n3c4ccccc4c4ccccc43)c(-c3ccc(N(c4cccc(-c5cccc6ccccc56)c4)c4cccc(-c5c(-c6ccccc6)ccc6ccccc56)c4)cc3)c2)cc1] 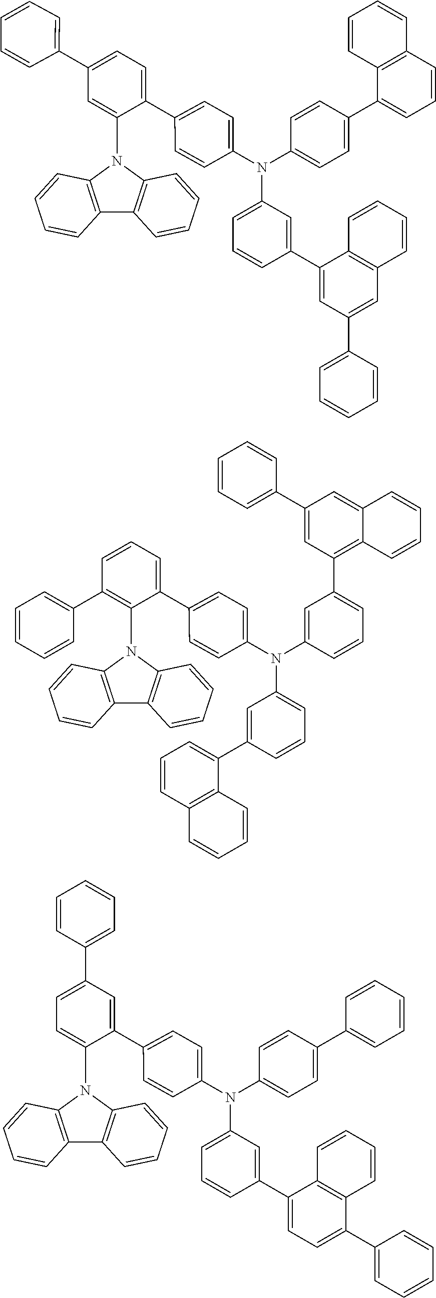 [SMILES: c1ccc(-c2ccc(-c3ccc(N(c4ccc(-c5cccc6ccccc56)cc4)c4cccc(-c5cc(-c6ccccc6)cc6ccccc56)c4)cc3)c(-n3c4ccccc4c4ccccc43)c2)cc1 ; c1ccc(-c2cc(-c3cccc(N(c4ccc(-c5cccc(-c6ccccc6)c5-n5c6ccccc6c6ccccc65)cc4)c4cccc(-c5cccc6ccccc56)c4)c3)c3ccccc3c2)cc1 ; c1ccc(-c2ccc(N(c3ccc(-c4cc(-c5ccccc5)ccc4-n4c5ccccc5c5ccccc54)cc3)c3cccc(-c4ccc(-c5ccccc5)c5ccccc45)c3)cc2)cc1] 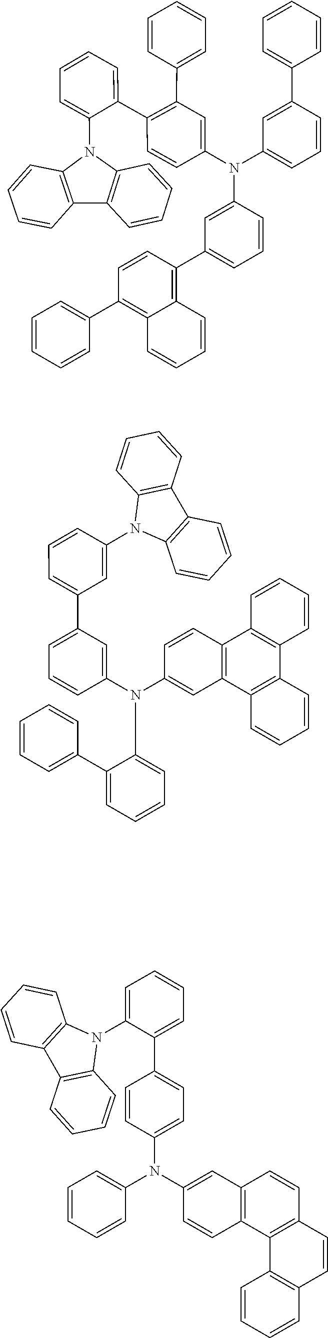 [SMILES: c1ccc(-c2cccc(N(c3cccc(-c4ccc(-c5ccccc5)c5ccccc45)c3)c3ccc(-c4ccccc4-n4c5ccccc5c5ccccc54)c(-c4ccccc4)c3)c2)cc1 ; c1ccc(-c2ccccc2N(c2cccc(-c3cccc(-n4c5ccccc5c5ccccc54)c3)c2)c2ccc3c4ccccc4c4ccccc4c3c2)cc1 ; c1ccc(N(c2ccc(-c3ccccc3-n3c4ccccc4c4ccccc43)cc2)c2ccc3c(ccc4ccc5ccccc5c43)c2)cc1] 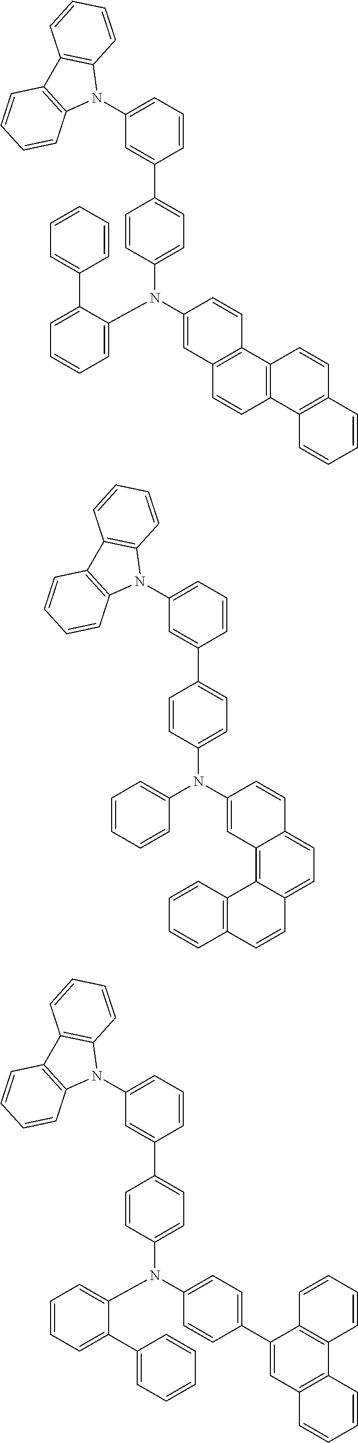 [SMILES: c1ccc(-c2ccccc2N(c2ccc(-c3cccc(-n4c5ccccc5c5ccccc54)c3)cc2)c2ccc3c(ccc4c5ccccc5ccc34)c2)cc1 ; c1ccc(N(c2ccc(-c3cccc(-n4c5ccccc5c5ccccc54)c3)cc2)c2ccc3ccc4ccc5ccccc5c4c3c2)cc1 ; c1ccc(-c2ccccc2N(c2ccc(-c3cccc(-n4c5ccccc5c5ccccc54)c3)cc2)c2ccc(-c3cc4ccccc4c4ccccc34)cc2)cc1] 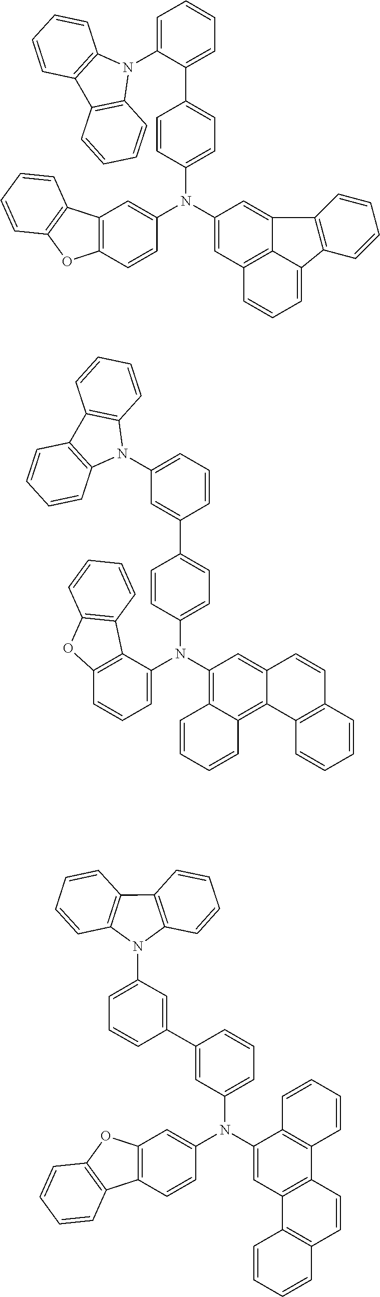 [SMILES: c1ccc2c(c1)-c1cccc3cc(N(c4ccc(-c5ccccc5-n5c6ccccc6c6ccccc65)cc4)c4ccc5oc6ccccc6c5c4)cc-2c13 ; c1cc(-c2ccc(N(c3cc4ccc5ccccc5c4c4ccccc34)c3cccc4oc5ccccc5c34)cc2)cc(-n2c3ccccc3c3ccccc32)c1 ; c1cc(-c2cccc(-n3c4ccccc4c4ccccc43)c2)cc(N(c2ccc3c(c2)oc2ccccc23)c2cc3c4ccccc4ccc3c3ccccc23)c1] 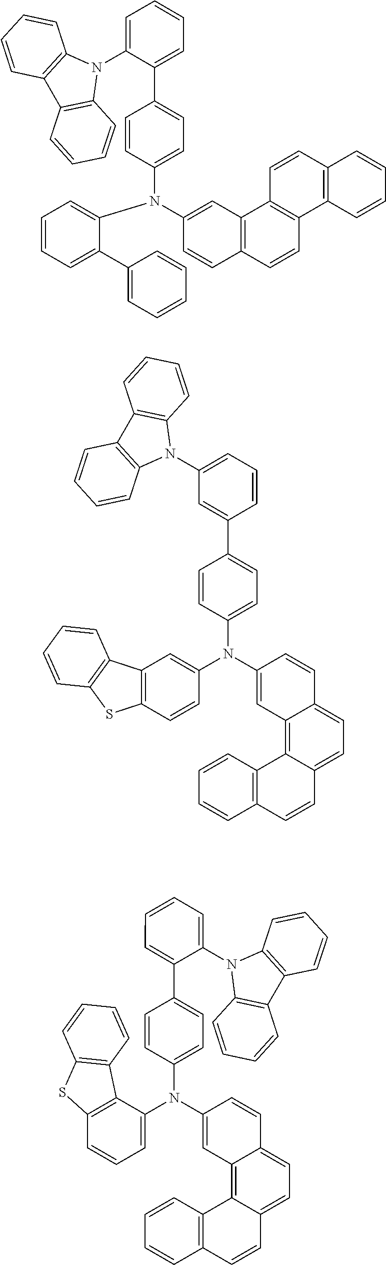 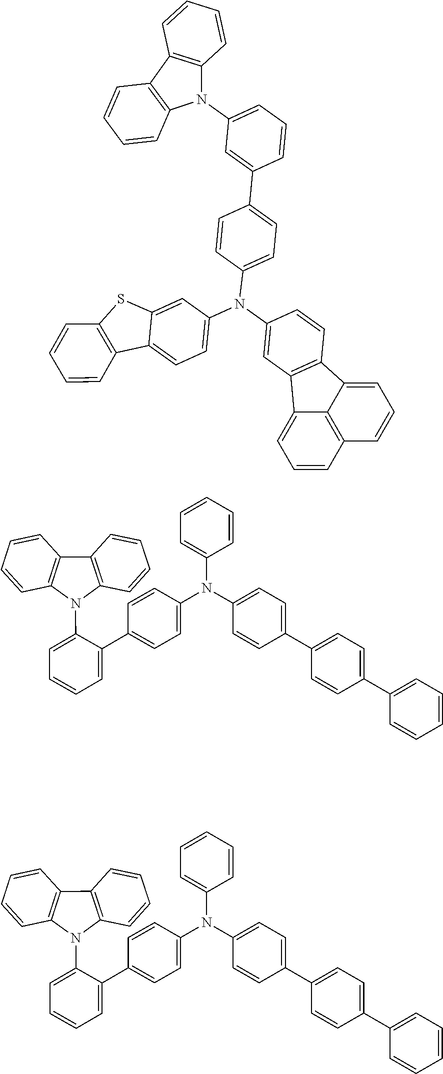 [SMILES: c1cc(-c2ccc(N(c3ccc4c(c3)-c3cccc5cccc-4c35)c3ccc4c(c3)sc3ccccc34)cc2)cc(-n2c3ccccc3c3ccccc32)c1 ; c1ccc(-c2ccc(-c3ccc(N(c4ccccc4)c4ccc(-c5ccccc5-n5c6ccccc6c6ccccc65)cc4)cc3)cc2)cc1 ; c1ccc(-c2ccc(-c3ccc(N(c4ccccc4)c4ccc(-c5ccccc5-n5c6ccccc6c6ccccc65)cc4)cc3)cc2)cc1] 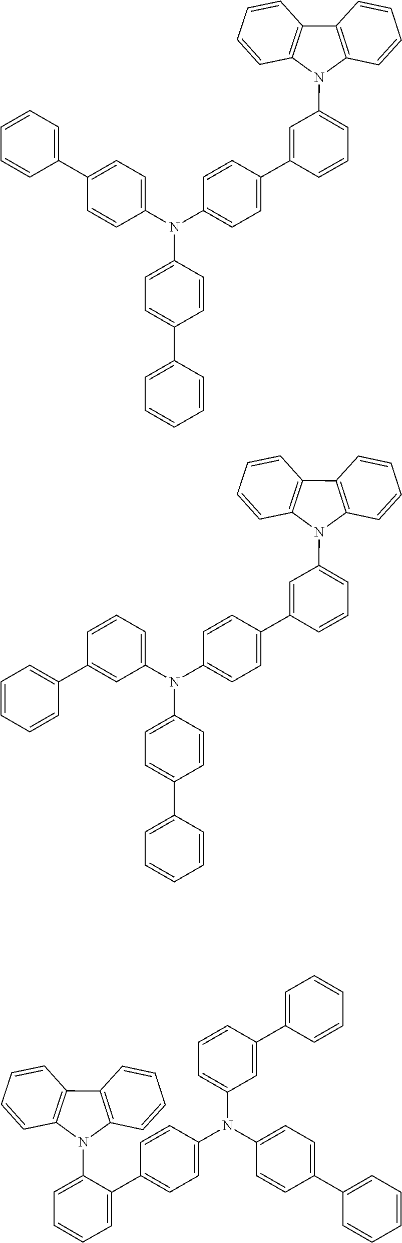 [SMILES: c1ccc(-c2ccc(N(c3ccc(-c4ccccc4)cc3)c3ccc(-c4cccc(-n5c6ccccc6c6ccccc65)c4)cc3)cc2)cc1 ; c1ccc(-c2ccc(N(c3ccc(-c4cccc(-n5c6ccccc6c6ccccc65)c4)cc3)c3cccc(-c4ccccc4)c3)cc2)cc1 ; c1ccc(-c2ccc(N(c3ccc(-c4ccccc4-n4c5ccccc5c5ccccc54)cc3)c3cccc(-c4ccccc4)c3)cc2)cc1] 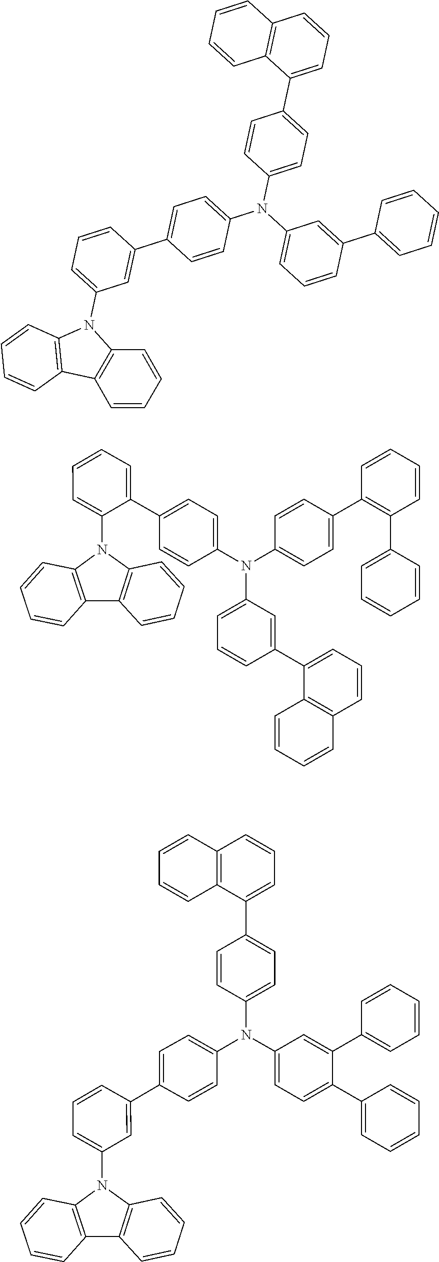 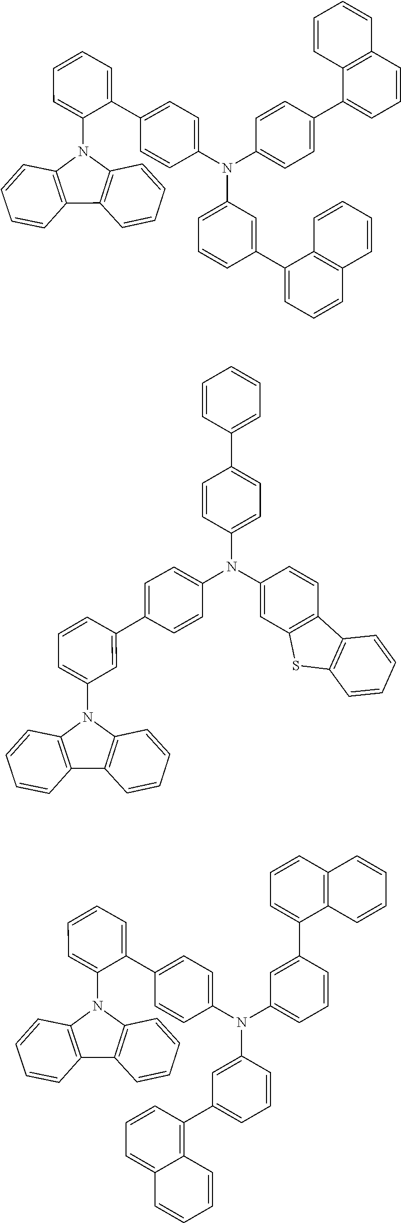 [SMILES: c1cc(-c2cccc3ccccc23)cc(N(c2ccc(-c3ccccc3-n3c4ccccc4c4ccccc43)cc2)c2ccc(-c3cccc4ccccc34)cc2)c1 ; c1ccc(-c2ccc(N(c3ccc(-c4cccc(-n5c6ccccc6c6ccccc65)c4)cc3)c3ccc4c(c3)sc3ccccc34)cc2)cc1 ; c1cc(-c2cccc3ccccc23)cc(N(c2ccc(-c3ccccc3-n3c4ccccc4c4ccccc43)cc2)c2cccc(-c3cccc4ccccc34)c2)c1] 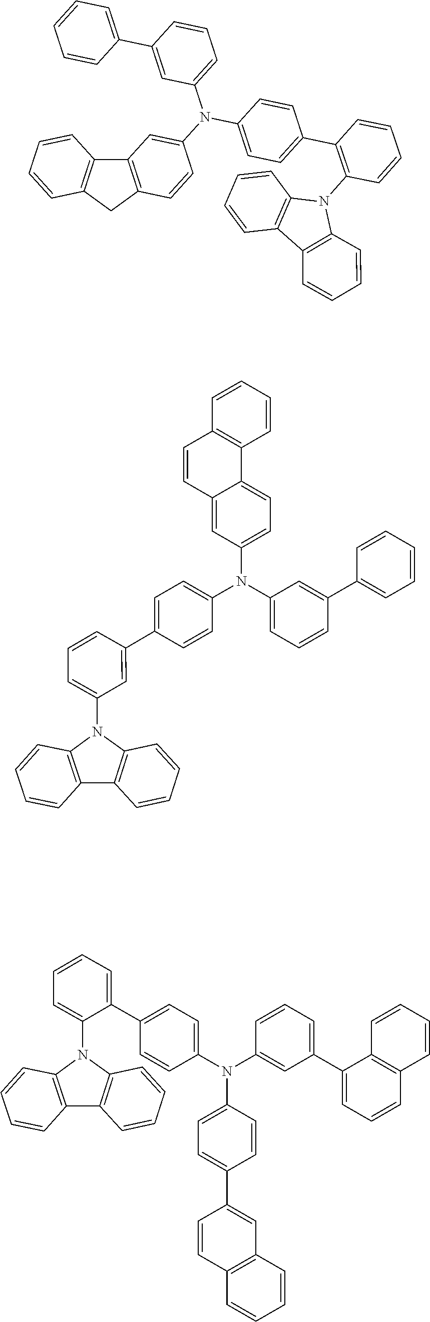 [SMILES: c1ccc(-c2cccc(N(c3ccc(-c4ccccc4-n4c5ccccc5c5ccccc54)cc3)c3ccc4c(c3)-c3ccccc3C4)c2)cc1 ; c1ccc(-c2cccc(N(c3ccc(-c4cccc(-n5c6ccccc6c6ccccc65)c4)cc3)c3ccc4c(ccc5ccccc54)c3)c2)cc1 ; c1cc(-c2cccc3ccccc23)cc(N(c2ccc(-c3ccc4ccccc4c3)cc2)c2ccc(-c3ccccc3-n3c4ccccc4c4ccccc43)cc2)c1] 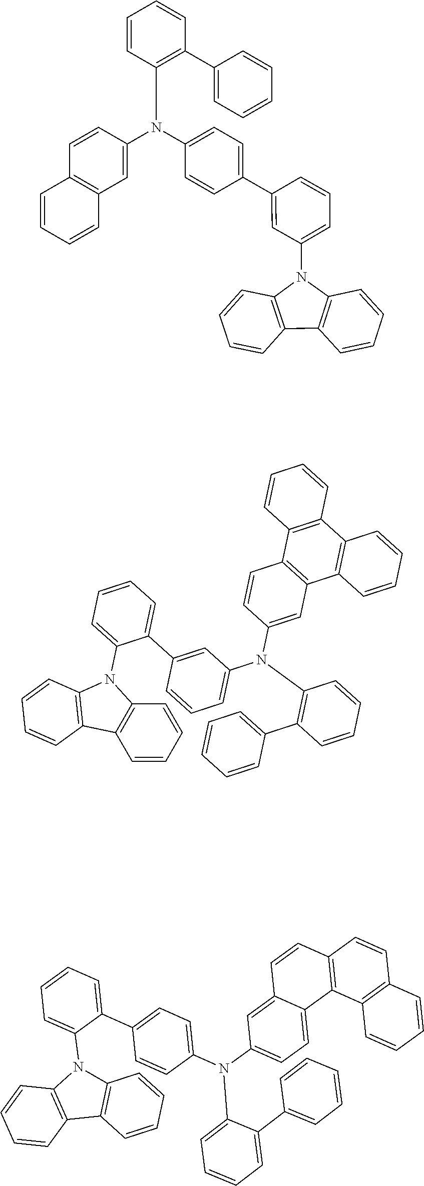 [SMILES: c1ccc(-c2ccccc2N(c2ccc(-c3cccc(-n4c5ccccc5c5ccccc54)c3)cc2)c2ccc3ccccc3c2)cc1 ; c1ccc(-c2ccccc2N(c2cccc(-c3ccccc3-n3c4ccccc4c4ccccc43)c2)c2ccc3c4ccccc4c4ccccc4c3c2)cc1 ; c1ccc(-c2ccccc2N(c2ccc(-c3ccccc3-n3c4ccccc4c4ccccc43)cc2)c2ccc3c(ccc4ccc5ccccc5c43)c2)cc1] 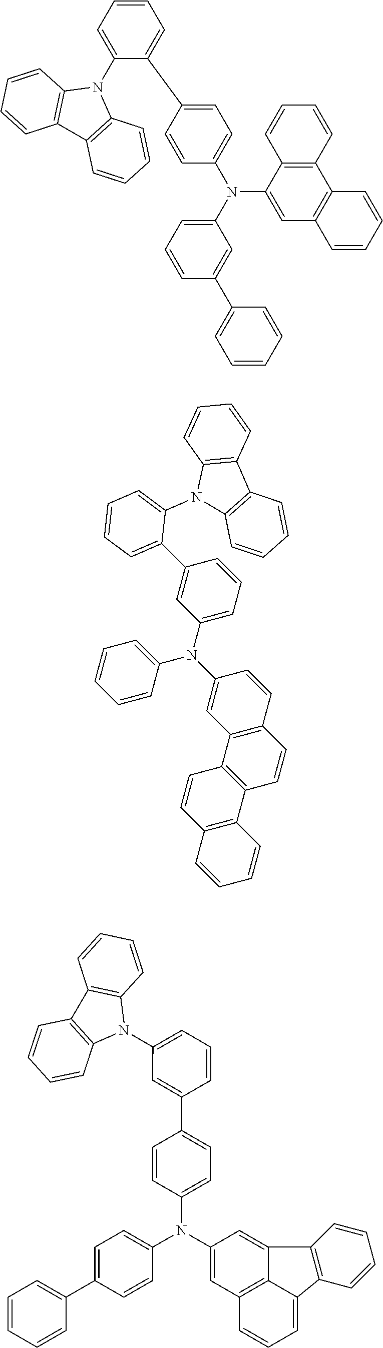 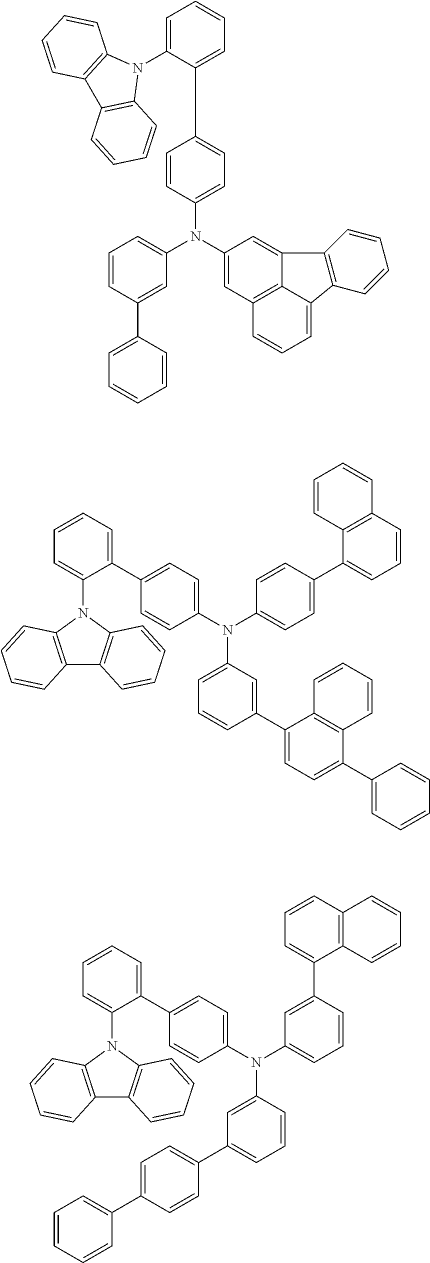 [SMILES: c1ccc(-c2cccc(N(c3ccc(-c4ccccc4-n4c5ccccc5c5ccccc54)cc3)c3cc4c5c(cccc5c3)-c3ccccc3-4)c2)cc1 ; c1ccc(-c2ccc(-c3cccc(N(c4ccc(-c5ccccc5-n5c6ccccc6c6ccccc65)cc4)c4ccc(-c5cccc6ccccc56)cc4)c3)c3ccccc23)cc1 ; c1ccc(-c2ccc(-c3cccc(N(c4ccc(-c5ccccc5-n5c6ccccc6c6ccccc65)cc4)c4cccc(-c5cccc6ccccc56)c4)c3)cc2)cc1] 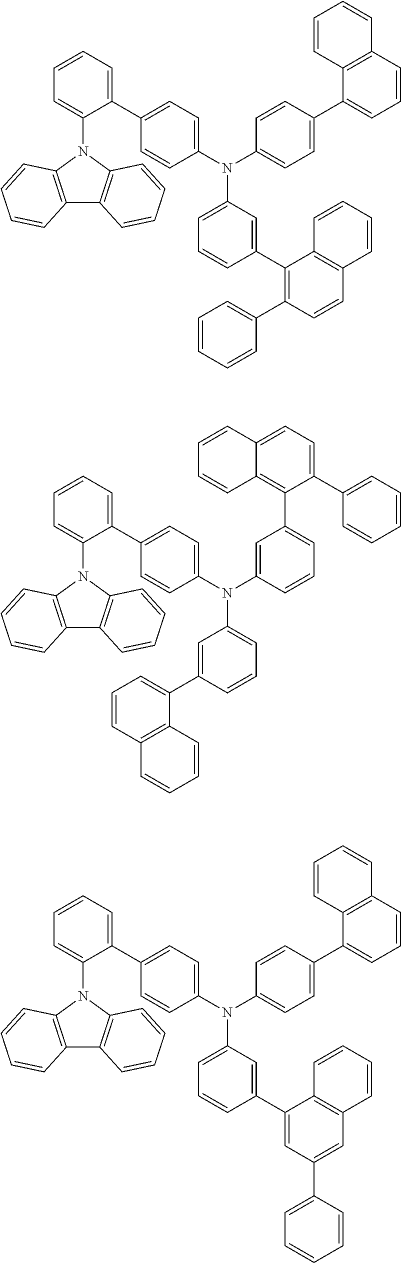 [SMILES: c1ccc(-c2ccc3ccccc3c2-c2cccc(N(c3ccc(-c4ccccc4-n4c5ccccc5c5ccccc54)cc3)c3ccc(-c4cccc5ccccc45)cc3)c2)cc1 ; c1ccc(-c2ccc3ccccc3c2-c2cccc(N(c3ccc(-c4ccccc4-n4c5ccccc5c5ccccc54)cc3)c3cccc(-c4cccc5ccccc45)c3)c2)cc1 ; c1ccc(-c2cc(-c3cccc(N(c4ccc(-c5ccccc5-n5c6ccccc6c6ccccc65)cc4)c4ccc(-c5cccc6ccccc56)cc4)c3)c3ccccc3c2)cc1] 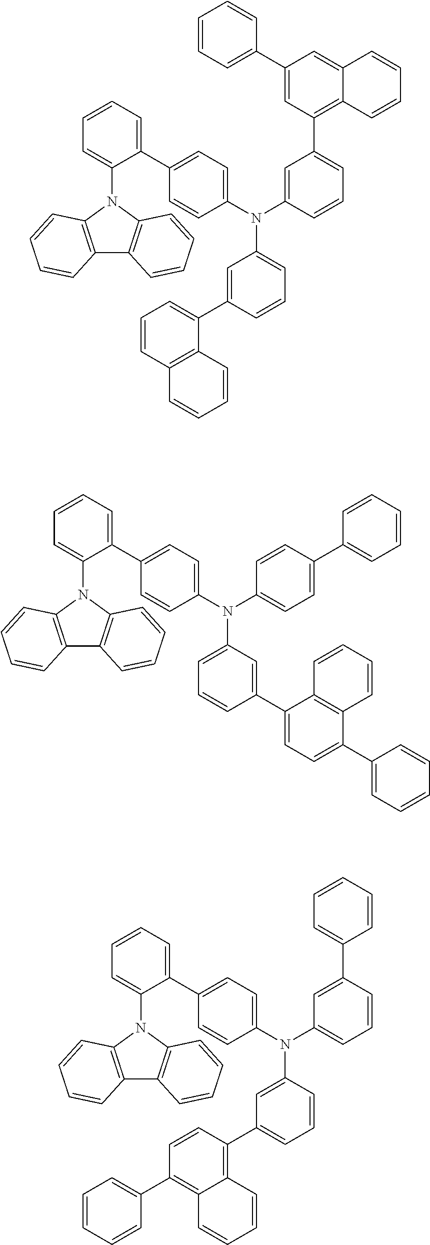 [SMILES: c1ccc(-c2cc(-c3cccc(N(c4ccc(-c5ccccc5-n5c6ccccc6c6ccccc65)cc4)c4cccc(-c5cccc6ccccc56)c4)c3)c3ccccc3c2)cc1 ; c1ccc(-c2ccc(N(c3ccc(-c4ccccc4-n4c5ccccc5c5ccccc54)cc3)c3cccc(-c4ccc(-c5ccccc5)c5ccccc45)c3)cc2)cc1 ; c1ccc(-c2cccc(N(c3ccc(-c4ccccc4-n4c5ccccc5c5ccccc54)cc3)c3cccc(-c4ccc(-c5ccccc5)c5ccccc45)c3)c2)cc1] 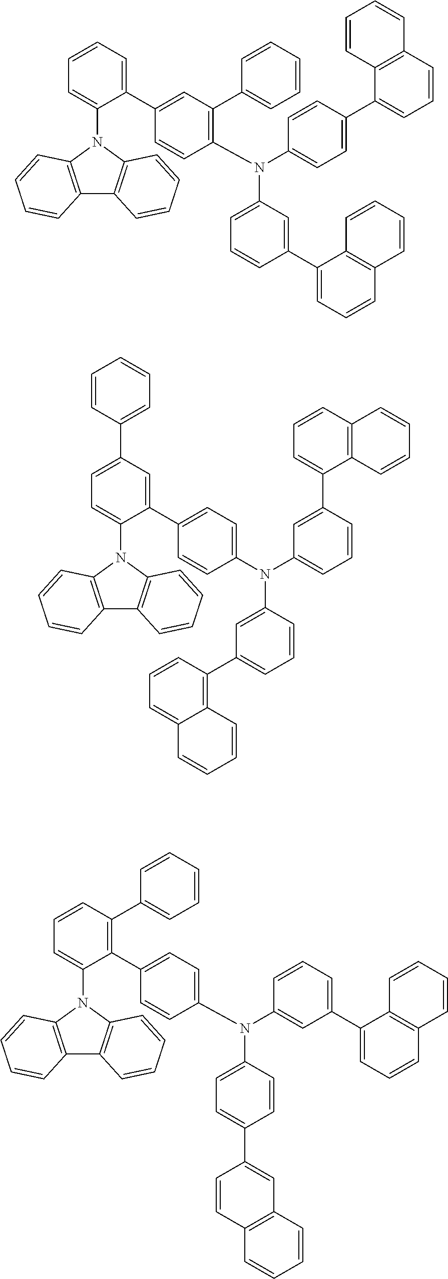 [SMILES: c1ccc(-c2cc(-c3ccccc3-n3c4ccccc4c4ccccc43)ccc2N(c2ccc(-c3cccc4ccccc34)cc2)c2cccc(-c3cccc4ccccc34)c2)cc1 ; c1ccc(-c2ccc(-n3c4ccccc4c4ccccc43)c(-c3ccc(N(c4cccc(-c5cccc6ccccc56)c4)c4cccc(-c5cccc6ccccc56)c4)cc3)c2)cc1 ; c1ccc(-c2cccc(-n3c4ccccc4c4ccccc43)c2-c2ccc(N(c3ccc(-c4ccc5ccccc5c4)cc3)c3cccc(-c4cccc5ccccc45)c3)cc2)cc1] 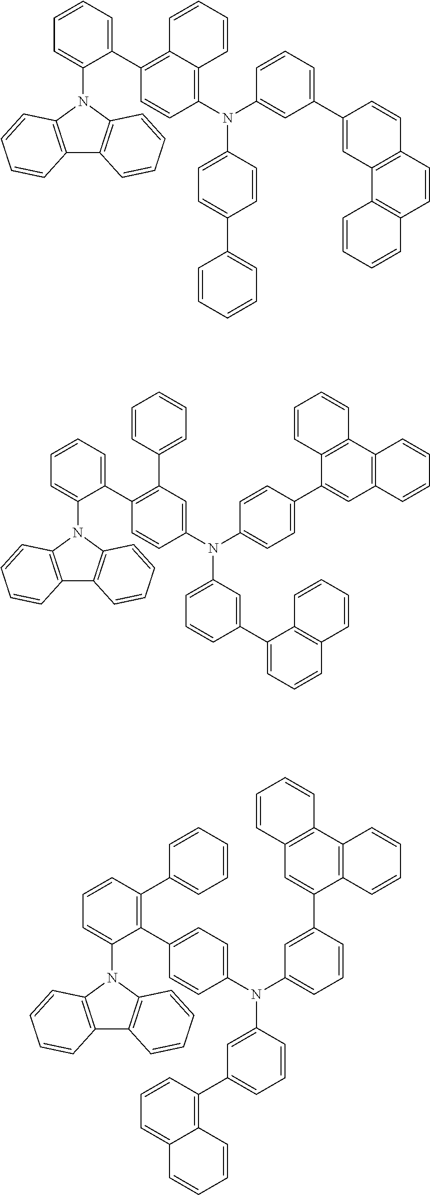 [SMILES: c1ccc(-c2ccc(N(c3cccc(-c4ccc5ccc6ccccc6c5c4)c3)c3ccc(-c4ccccc4-n4c5ccccc5c5ccccc54)c4ccccc34)cc2)cc1 ; c1ccc(-c2cc(N(c3ccc(-c4cc5ccccc5c5ccccc45)cc3)c3cccc(-c4cccc5ccccc45)c3)ccc2-c2ccccc2-n2c3ccccc3c3ccccc32)cc1 ; c1ccc(-c2cccc(-n3c4ccccc4c4ccccc43)c2-c2ccc(N(c3cccc(-c4cccc5ccccc45)c3)c3cccc(-c4cc5ccccc5c5ccccc45)c3)cc2)cc1] 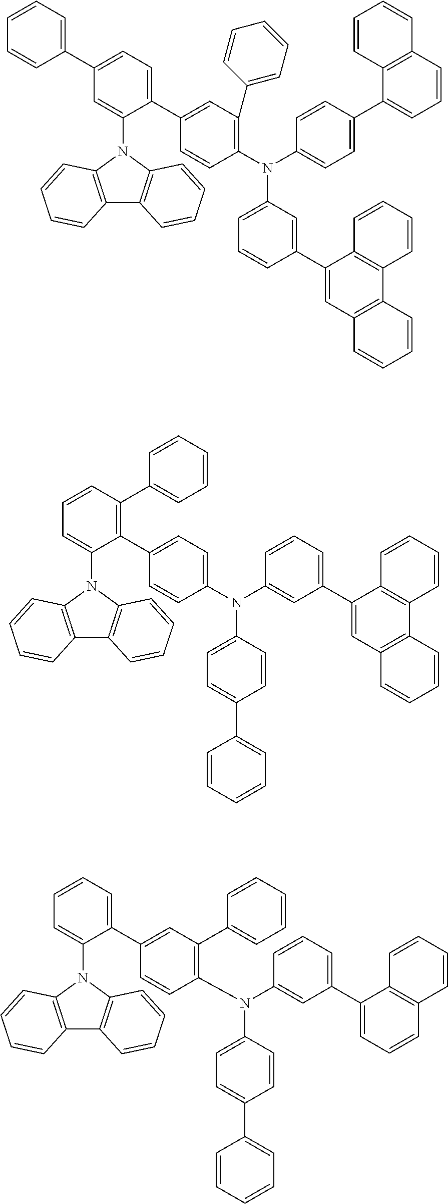 [SMILES: c1ccc(-c2ccc(-c3ccc(N(c4ccc(-c5cccc6ccccc56)cc4)c4cccc(-c5cc6ccccc6c6ccccc56)c4)c(-c4ccccc4)c3)c(-n3c4ccccc4c4ccccc43)c2)cc1 ; c1ccc(-c2ccc(N(c3ccc(-c4c(-c5ccccc5)cccc4-n4c5ccccc5c5ccccc54)cc3)c3cccc(-c4cc5ccccc5c5ccccc45)c3)cc2)cc1 ; c1ccc(-c2ccc(N(c3cccc(-c4cccc5ccccc45)c3)c3ccc(-c4ccccc4-n4c5ccccc5c5ccccc54)cc3-c3ccccc3)cc2)cc1] 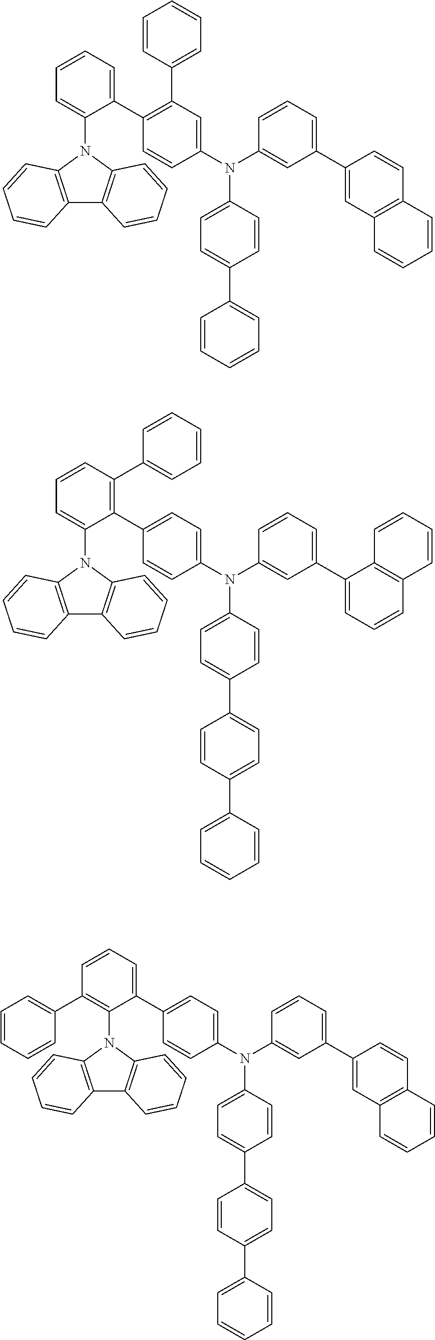 [SMILES: c1ccc(-c2ccc(N(c3cccc(-c4ccc5ccccc5c4)c3)c3ccc(-c4ccccc4-n4c5ccccc5c5ccccc54)c(-c4ccccc4)c3)cc2)cc1 ; c1ccc(-c2ccc(-c3ccc(N(c4ccc(-c5c(-c6ccccc6)cccc5-n5c6ccccc6c6ccccc65)cc4)c4cccc(-c5cccc6ccccc56)c4)cc3)cc2)cc1 ; c1ccc(-c2ccc(-c3ccc(N(c4ccc(-c5cccc(-c6ccccc6)c5-n5c6ccccc6c6ccccc65)cc4)c4cccc(-c5ccc6ccccc6c5)c4)cc3)cc2)cc1] 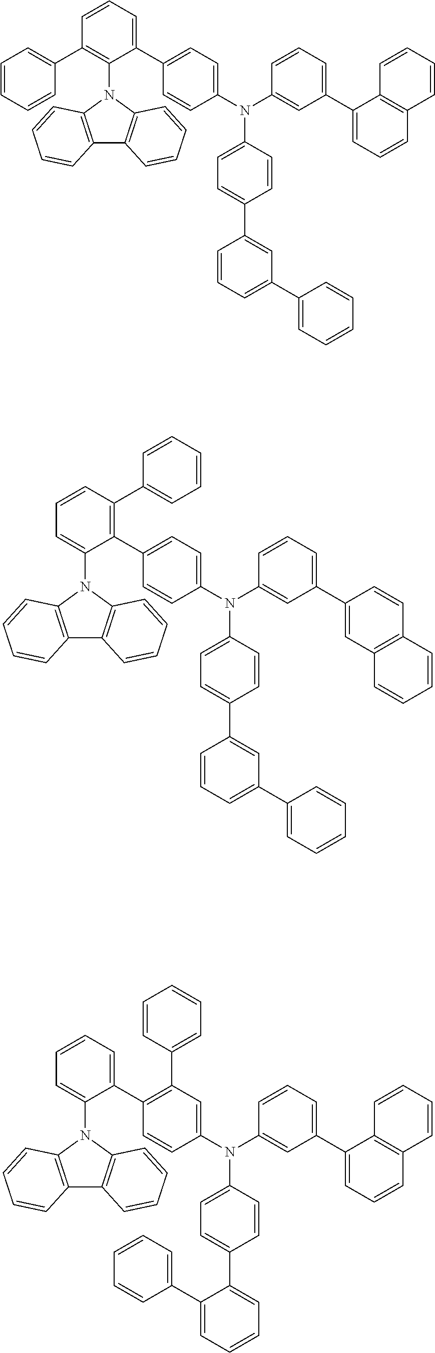 [SMILES: c1ccc(-c2cccc(-c3ccc(N(c4ccc(-c5cccc(-c6ccccc6)c5-n5c6ccccc6c6ccccc65)cc4)c4cccc(-c5cccc6ccccc56)c4)cc3)c2)cc1 ; c1ccc(-c2cccc(-c3ccc(N(c4ccc(-c5c(-c6ccccc6)cccc5-n5c6ccccc6c6ccccc65)cc4)c4cccc(-c5ccc6ccccc6c5)c4)cc3)c2)cc1 ; c1ccc(-c2ccccc2-c2ccc(N(c3cccc(-c4cccc5ccccc45)c3)c3ccc(-c4ccccc4-n4c5ccccc5c5ccccc54)c(-c4ccccc4)c3)cc2)cc1] 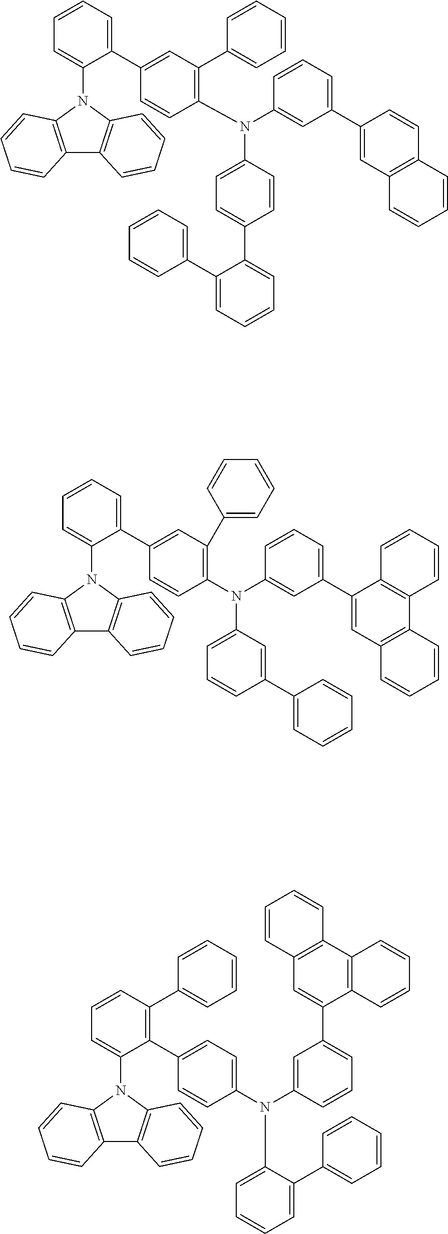 [SMILES: c1ccc(-c2ccccc2-c2ccc(N(c3cccc(-c4ccc5ccccc5c4)c3)c3ccc(-c4ccccc4-n4c5ccccc5c5ccccc54)cc3-c3ccccc3)cc2)cc1 ; c1ccc(-c2cccc(N(c3cccc(-c4cc5ccccc5c5ccccc45)c3)c3ccc(-c4ccccc4-n4c5ccccc5c5ccccc54)cc3-c3ccccc3)c2)cc1 ; c1ccc(-c2ccccc2N(c2ccc(-c3c(-c4ccccc4)cccc3-n3c4ccccc4c4ccccc43)cc2)c2cccc(-c3cc4ccccc4c4ccccc34)c2)cc1] 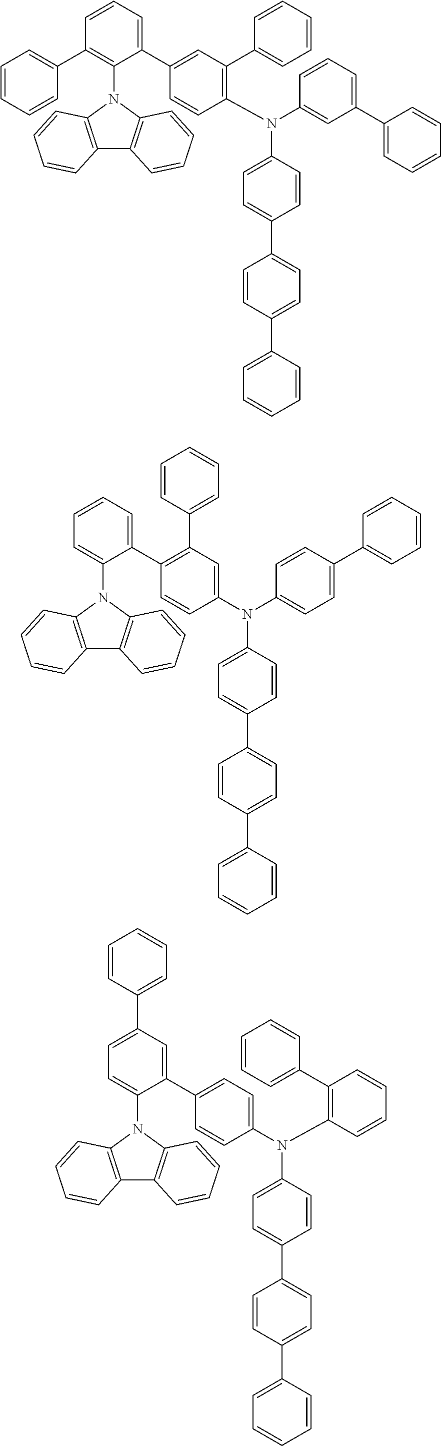 [SMILES: c1ccc(-c2ccc(-c3ccc(N(c4cccc(-c5ccccc5)c4)c4ccc(-c5cccc(-c6ccccc6)c5-n5c6ccccc6c6ccccc65)cc4-c4ccccc4)cc3)cc2)cc1 ; c1ccc(-c2ccc(-c3ccc(N(c4ccc(-c5ccccc5)cc4)c4ccc(-c5ccccc5-n5c6ccccc6c6ccccc65)c(-c5ccccc5)c4)cc3)cc2)cc1 ; c1ccc(-c2ccc(-c3ccc(N(c4ccc(-c5cc(-c6ccccc6)ccc5-n5c6ccccc6c6ccccc65)cc4)c4ccccc4-c4ccccc4)cc3)cc2)cc1] 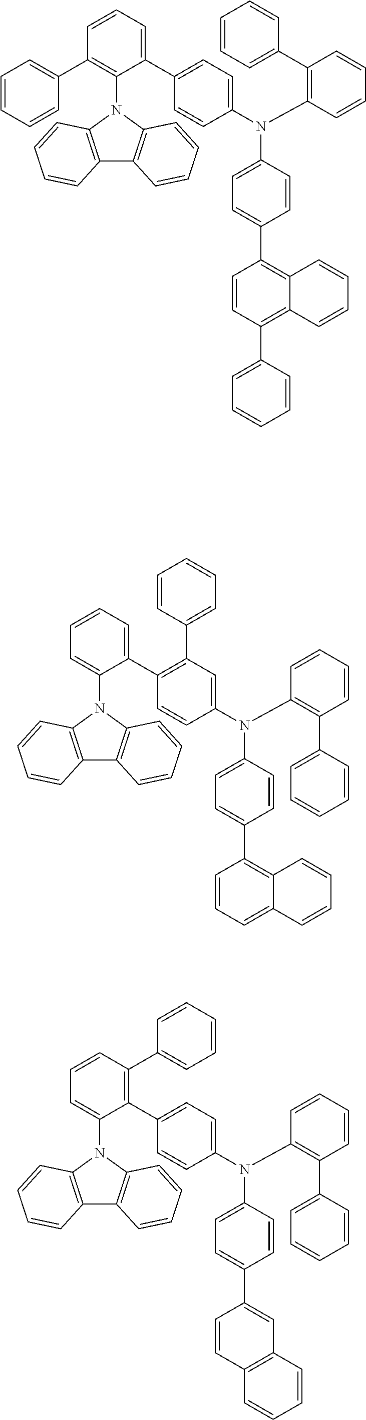 [SMILES: c1ccc(-c2ccccc2N(c2ccc(-c3cccc(-c4ccccc4)c3-n3c4ccccc4c4ccccc43)cc2)c2ccc(-c3ccc(-c4ccccc4)c4ccccc34)cc2)cc1 ; c1ccc(-c2cc(N(c3ccc(-c4cccc5ccccc45)cc3)c3ccccc3-c3ccccc3)ccc2-c2ccccc2-n2c3ccccc3c3ccccc32)cc1 ; c1ccc(-c2ccccc2N(c2ccc(-c3ccc4ccccc4c3)cc2)c2ccc(-c3c(-c4ccccc4)cccc3-n3c4ccccc4c4ccccc43)cc2)cc1] 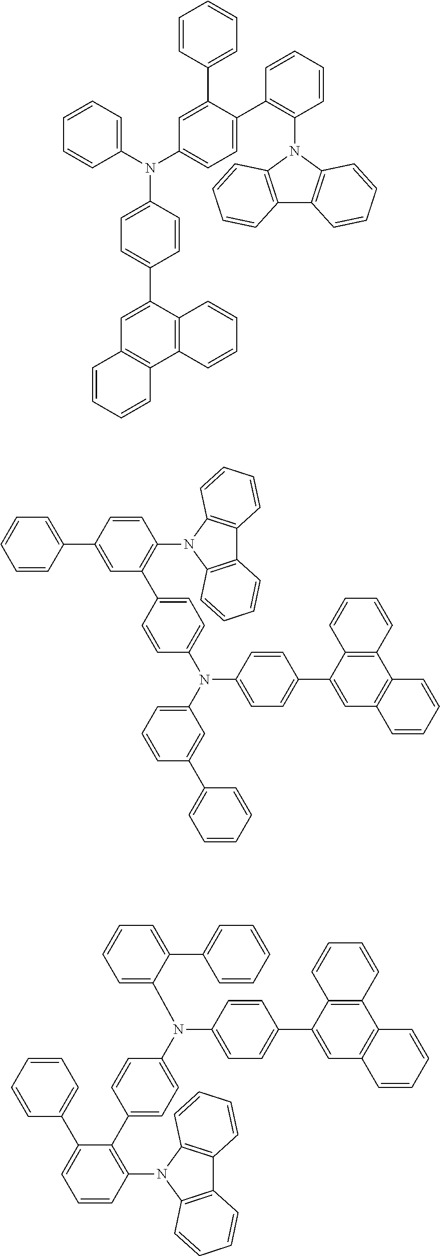 [SMILES: c1ccc(-c2cc(N(c3ccccc3)c3ccc(-c4cc5ccccc5c5ccccc45)cc3)ccc2-c2ccccc2-n2c3ccccc3c3ccccc32)cc1 ; c1ccc(-c2cccc(N(c3ccc(-c4cc(-c5ccccc5)ccc4-n4c5ccccc5c5ccccc54)cc3)c3ccc(-c4cc5ccccc5c5ccccc45)cc3)c2)cc1 ; c1ccc(-c2ccccc2N(c2ccc(-c3c(-c4ccccc4)cccc3-n3c4ccccc4c4ccccc43)cc2)c2ccc(-c3cc4ccccc4c4ccccc34)cc2)cc1] 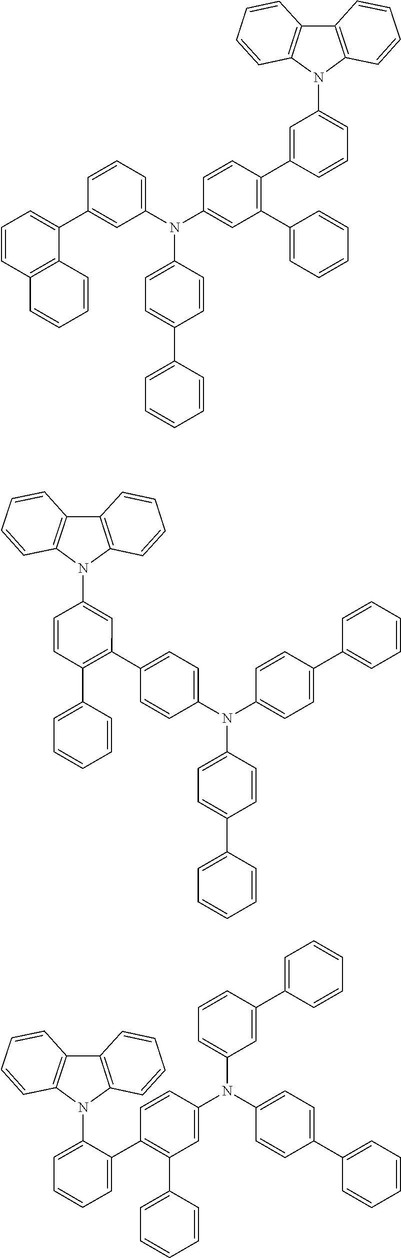 [SMILES: c1ccc(-c2ccc(N(c3cccc(-c4cccc5ccccc45)c3)c3ccc(-c4cccc(-n5c6ccccc6c6ccccc65)c4)c(-c4ccccc4)c3)cc2)cc1 ; c1ccc(-c2ccc(N(c3ccc(-c4ccccc4)cc3)c3ccc(-c4cc(-n5c6ccccc6c6ccccc65)ccc4-c4ccccc4)cc3)cc2)cc1 ; c1ccc(-c2ccc(N(c3cccc(-c4ccccc4)c3)c3ccc(-c4ccccc4-n4c5ccccc5c5ccccc54)c(-c4ccccc4)c3)cc2)cc1] 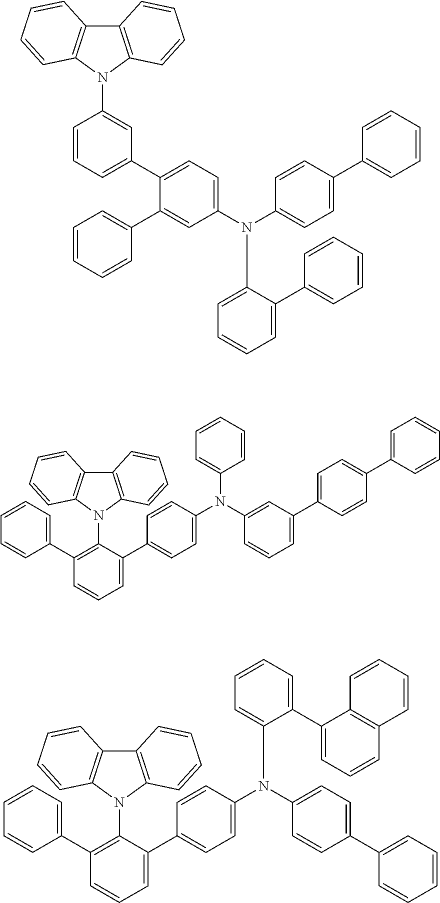 [SMILES: c1ccc(-c2ccc(N(c3ccc(-c4cccc(-n5c6ccccc6c6ccccc65)c4)c(-c4ccccc4)c3)c3ccccc3-c3ccccc3)cc2)cc1 ; c1ccc(-c2ccc(-c3cccc(N(c4ccccc4)c4ccc(-c5cccc(-c6ccccc6)c5-n5c6ccccc6c6ccccc65)cc4)c3)cc2)cc1 ; c1ccc(-c2ccc(N(c3ccc(-c4cccc(-c5ccccc5)c4-n4c5ccccc5c5ccccc54)cc3)c3ccccc3-c3cccc4ccccc34)cc2)cc1] 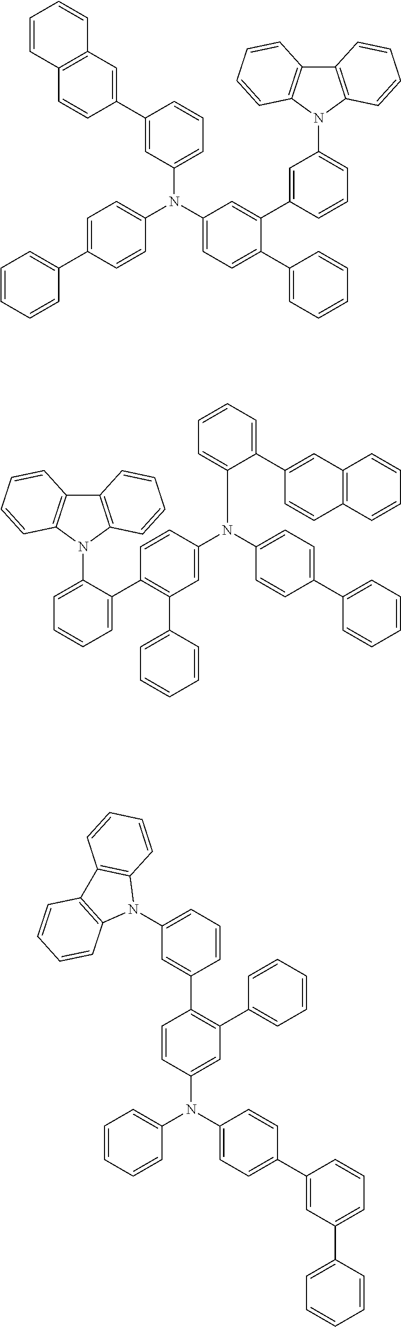 [SMILES: c1ccc(-c2ccc(N(c3cccc(-c4ccc5ccccc5c4)c3)c3ccc(-c4ccccc4)c(-c4cccc(-n5c6ccccc6c6ccccc65)c4)c3)cc2)cc1 ; c1ccc(-c2ccc(N(c3ccc(-c4ccccc4-n4c5ccccc5c5ccccc54)c(-c4ccccc4)c3)c3ccccc3-c3ccc4ccccc4c3)cc2)cc1 ; c1ccc(-c2cccc(-c3ccc(N(c4ccccc4)c4ccc(-c5cccc(-n6c7ccccc7c7ccccc76)c5)c(-c5ccccc5)c4)cc3)c2)cc1] 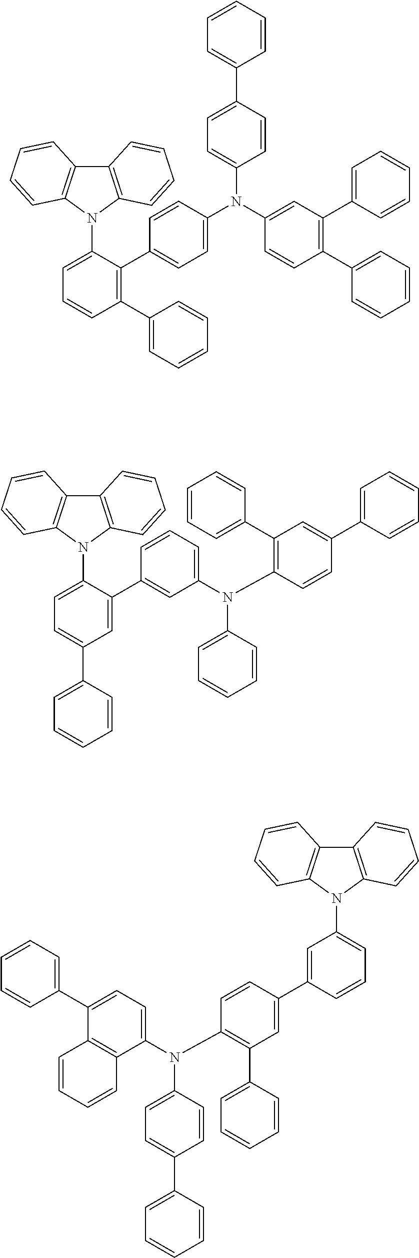 [SMILES: c1ccc(-c2ccc(N(c3ccc(-c4c(-c5ccccc5)cccc4-n4c5ccccc5c5ccccc54)cc3)c3ccc(-c4ccccc4)c(-c4ccccc4)c3)cc2)cc1 ; c1ccc(-c2ccc(N(c3ccccc3)c3cccc(-c4cc(-c5ccccc5)ccc4-n4c5ccccc5c5ccccc54)c3)c(-c3ccccc3)c2)cc1 ; c1ccc(-c2ccc(N(c3ccc(-c4cccc(-n5c6ccccc6c6ccccc65)c4)cc3-c3ccccc3)c3ccc(-c4ccccc4)c4ccccc34)cc2)cc1] 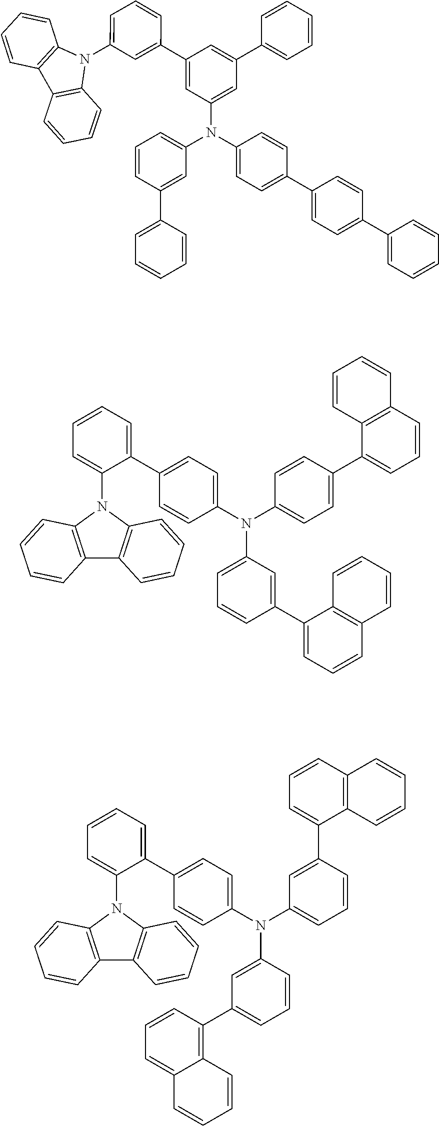 [SMILES: c1ccc(-c2ccc(-c3ccc(N(c4cccc(-c5ccccc5)c4)c4cc(-c5ccccc5)cc(-c5cccc(-n6c7ccccc7c7ccccc76)c5)c4)cc3)cc2)cc1 ; c1cc(-c2cccc3ccccc23)cc(N(c2ccc(-c3ccccc3-n3c4ccccc4c4ccccc43)cc2)c2ccc(-c3cccc4ccccc34)cc2)c1 ; c1cc(-c2cccc3ccccc23)cc(N(c2ccc(-c3ccccc3-n3c4ccccc4c4ccccc43)cc2)c2cccc(-c3cccc4ccccc34)c2)c1] 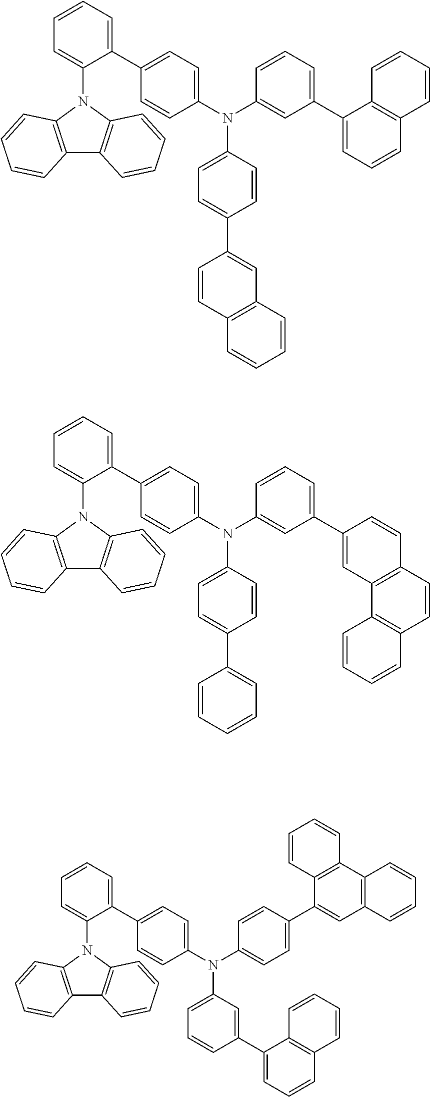 [SMILES: c1cc(-c2cccc3ccccc23)cc(N(c2ccc(-c3ccc4ccccc4c3)cc2)c2ccc(-c3ccccc3-n3c4ccccc4c4ccccc43)cc2)c1 ; c1ccc(-c2ccc(N(c3ccc(-c4ccccc4-n4c5ccccc5c5ccccc54)cc3)c3cccc(-c4ccc5ccc6ccccc6c5c4)c3)cc2)cc1 ; c1cc(-c2cccc3ccccc23)cc(N(c2ccc(-c3ccccc3-n3c4ccccc4c4ccccc43)cc2)c2ccc(-c3cc4ccccc4c4ccccc34)cc2)c1] 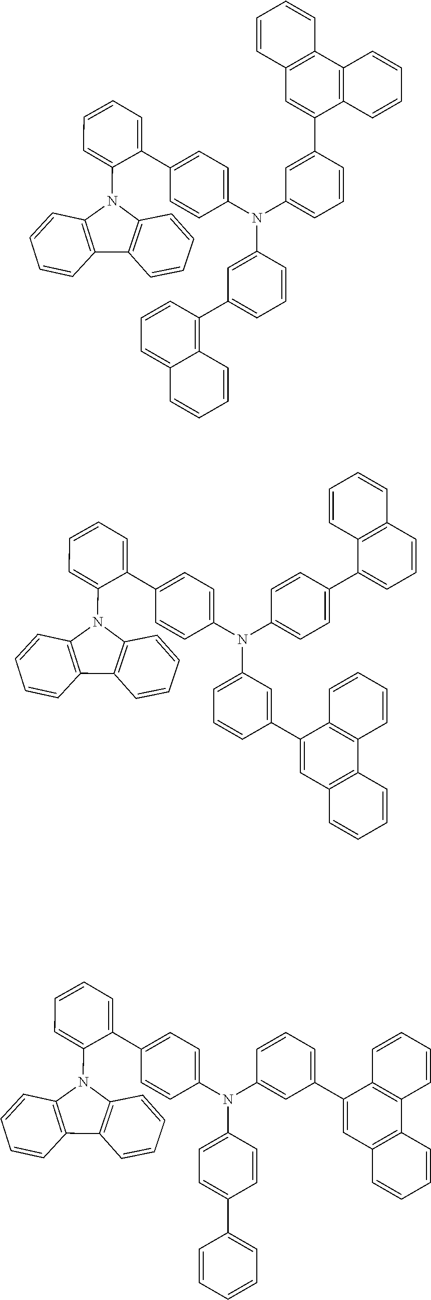 [SMILES: c1cc(-c2cccc3ccccc23)cc(N(c2ccc(-c3ccccc3-n3c4ccccc4c4ccccc43)cc2)c2cccc(-c3cc4ccccc4c4ccccc34)c2)c1 ; c1cc(-c2cc3ccccc3c3ccccc23)cc(N(c2ccc(-c3ccccc3-n3c4ccccc4c4ccccc43)cc2)c2ccc(-c3cccc4ccccc34)cc2)c1 ; c1ccc(-c2ccc(N(c3ccc(-c4ccccc4-n4c5ccccc5c5ccccc54)cc3)c3cccc(-c4cc5ccccc5c5ccccc45)c3)cc2)cc1] 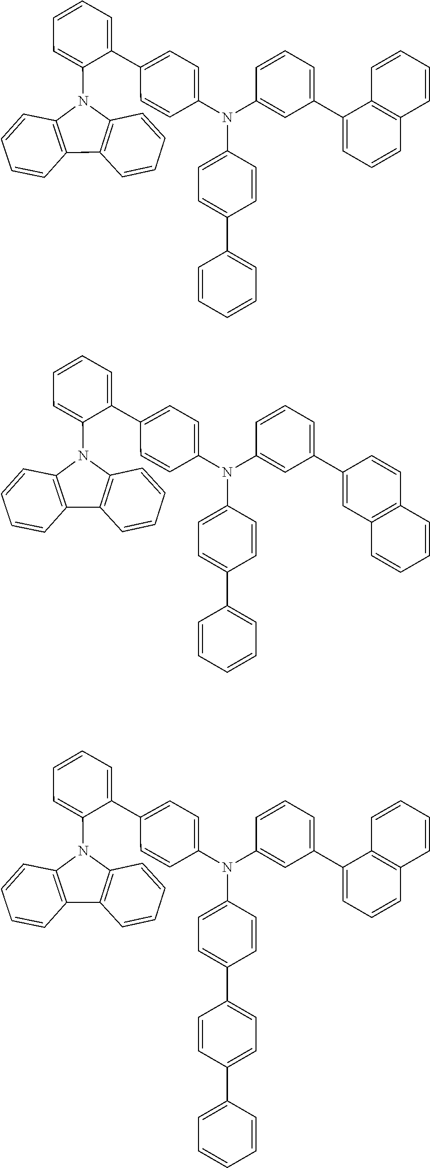 [SMILES: c1ccc(-c2ccc(N(c3ccc(-c4ccccc4-n4c5ccccc5c5ccccc54)cc3)c3cccc(-c4cccc5ccccc45)c3)cc2)cc1 ; c1ccc(-c2ccc(N(c3ccc(-c4ccccc4-n4c5ccccc5c5ccccc54)cc3)c3cccc(-c4ccc5ccccc5c4)c3)cc2)cc1 ; c1ccc(-c2ccc(-c3ccc(N(c4ccc(-c5ccccc5-n5c6ccccc6c6ccccc65)cc4)c4cccc(-c5cccc6ccccc56)c4)cc3)cc2)cc1] 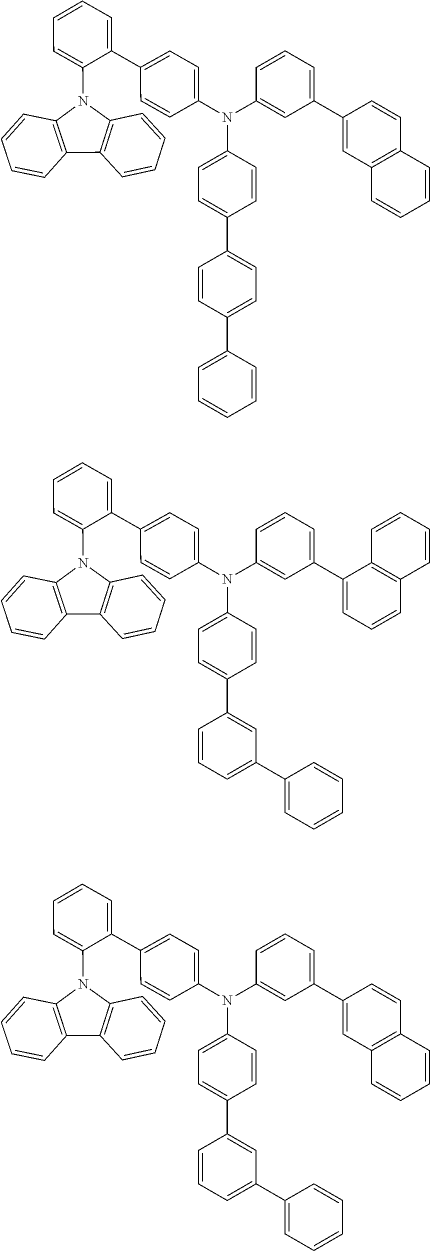 [SMILES: c1ccc(-c2ccc(-c3ccc(N(c4ccc(-c5ccccc5-n5c6ccccc6c6ccccc65)cc4)c4cccc(-c5ccc6ccccc6c5)c4)cc3)cc2)cc1 ; c1ccc(-c2cccc(-c3ccc(N(c4ccc(-c5ccccc5-n5c6ccccc6c6ccccc65)cc4)c4cccc(-c5cccc6ccccc56)c4)cc3)c2)cc1 ; c1ccc(-c2cccc(-c3ccc(N(c4ccc(-c5ccccc5-n5c6ccccc6c6ccccc65)cc4)c4cccc(-c5ccc6ccccc6c5)c4)cc3)c2)cc1] 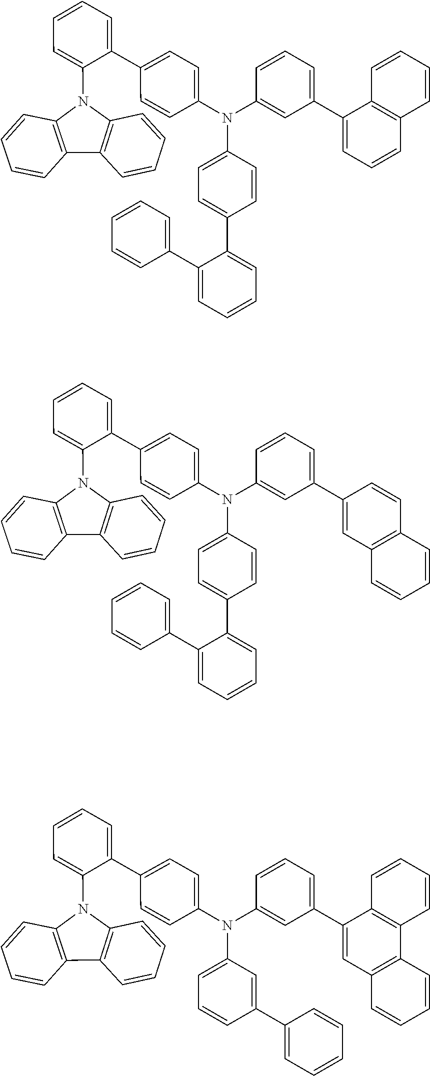 [SMILES: c1ccc(-c2ccccc2-c2ccc(N(c3ccc(-c4ccccc4-n4c5ccccc5c5ccccc54)cc3)c3cccc(-c4cccc5ccccc45)c3)cc2)cc1 ; c1ccc(-c2ccccc2-c2ccc(N(c3ccc(-c4ccccc4-n4c5ccccc5c5ccccc54)cc3)c3cccc(-c4ccc5ccccc5c4)c3)cc2)cc1 ; c1ccc(-c2cccc(N(c3ccc(-c4ccccc4-n4c5ccccc5c5ccccc54)cc3)c3cccc(-c4cc5ccccc5c5ccccc45)c3)c2)cc1] 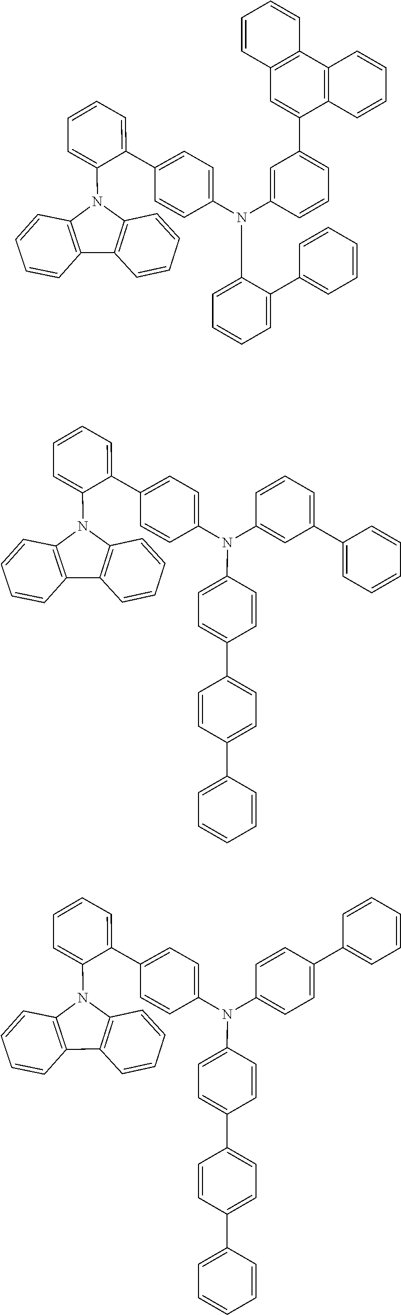 [SMILES: c1ccc(-c2ccccc2N(c2ccc(-c3ccccc3-n3c4ccccc4c4ccccc43)cc2)c2cccc(-c3cc4ccccc4c4ccccc34)c2)cc1 ; c1ccc(-c2ccc(-c3ccc(N(c4ccc(-c5ccccc5-n5c6ccccc6c6ccccc65)cc4)c4cccc(-c5ccccc5)c4)cc3)cc2)cc1 ; c1ccc(-c2ccc(-c3ccc(N(c4ccc(-c5ccccc5)cc4)c4ccc(-c5ccccc5-n5c6ccccc6c6ccccc65)cc4)cc3)cc2)cc1] 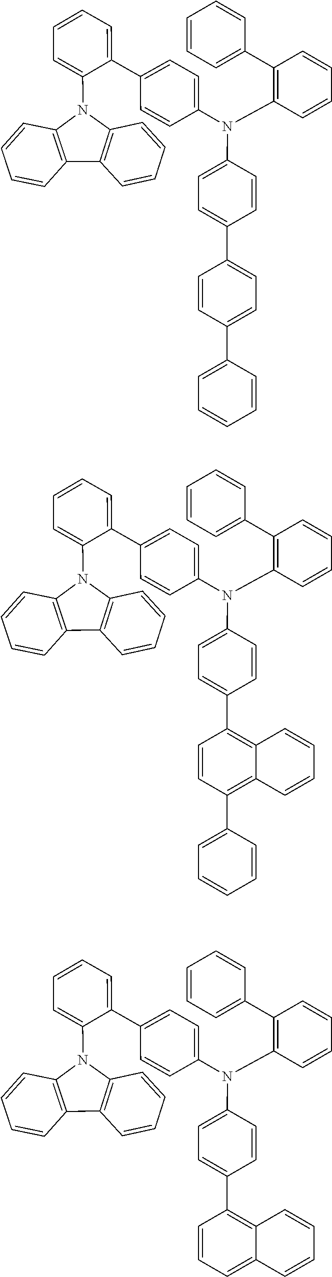 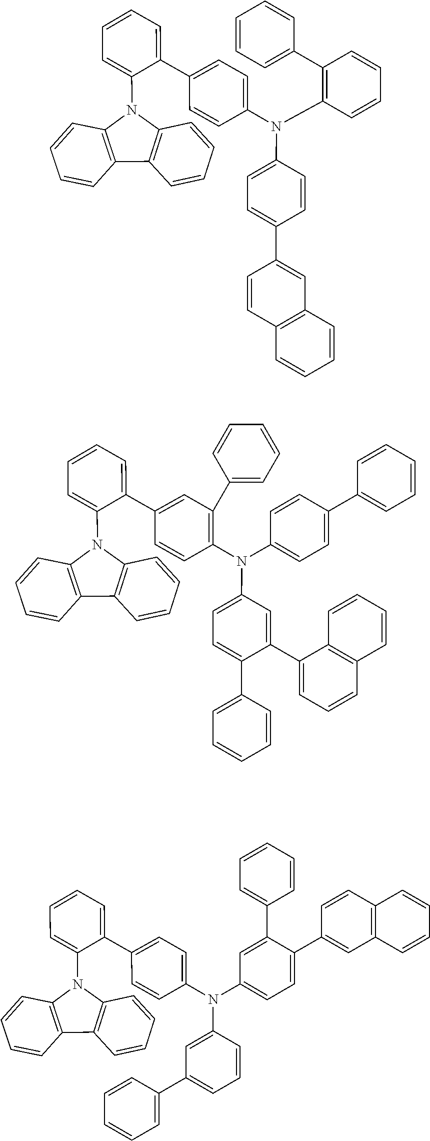 [SMILES: c1ccc(-c2ccccc2N(c2ccc(-c3ccc4ccccc4c3)cc2)c2ccc(-c3ccccc3-n3c4ccccc4c4ccccc43)cc2)cc1 ; c1ccc(-c2ccc(N(c3ccc(-c4ccccc4)c(-c4cccc5ccccc45)c3)c3ccc(-c4ccccc4-n4c5ccccc5c5ccccc54)cc3-c3ccccc3)cc2)cc1 ; c1ccc(-c2cccc(N(c3ccc(-c4ccccc4-n4c5ccccc5c5ccccc54)cc3)c3ccc(-c4ccc5ccccc5c4)c(-c4ccccc4)c3)c2)cc1] 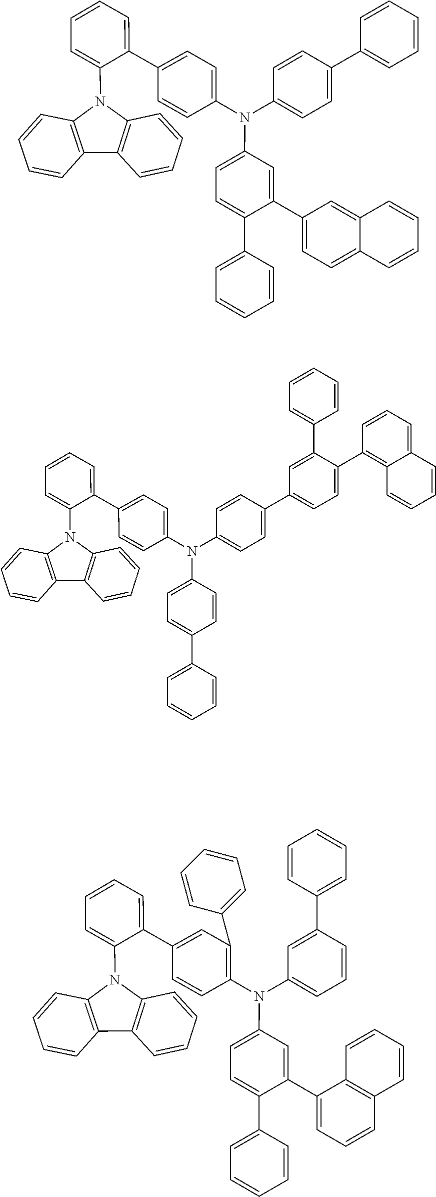 [SMILES: c1ccc(-c2ccc(N(c3ccc(-c4ccccc4-n4c5ccccc5c5ccccc54)cc3)c3ccc(-c4ccccc4)c(-c4ccc5ccccc5c4)c3)cc2)cc1 ; c1ccc(-c2ccc(N(c3ccc(-c4ccc(-c5cccc6ccccc56)c(-c5ccccc5)c4)cc3)c3ccc(-c4ccccc4-n4c5ccccc5c5ccccc54)cc3)cc2)cc1 ; c1ccc(-c2cccc(N(c3ccc(-c4ccccc4)c(-c4cccc5ccccc45)c3)c3ccc(-c4ccccc4-n4c5ccccc5c5ccccc54)cc3-c3ccccc3)c2)cc1] 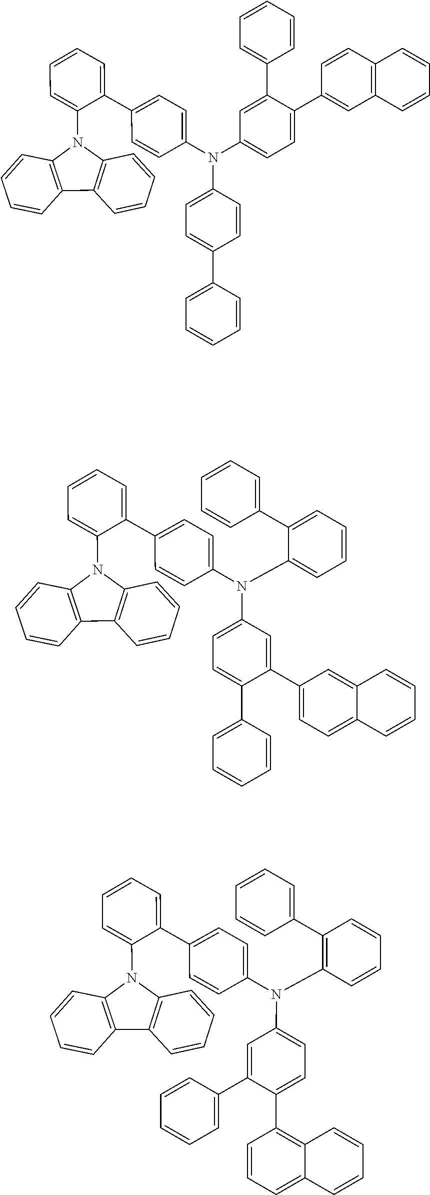 [SMILES: c1ccc(-c2ccc(N(c3ccc(-c4ccccc4-n4c5ccccc5c5ccccc54)cc3)c3ccc(-c4ccc5ccccc5c4)c(-c4ccccc4)c3)cc2)cc1 ; c1ccc(-c2ccc(N(c3ccc(-c4ccccc4-n4c5ccccc5c5ccccc54)cc3)c3ccccc3-c3ccccc3)cc2-c2ccc3ccccc3c2)cc1 ; c1ccc(-c2cc(N(c3ccc(-c4ccccc4-n4c5ccccc5c5ccccc54)cc3)c3ccccc3-c3ccccc3)ccc2-c2cccc3ccccc23)cc1] 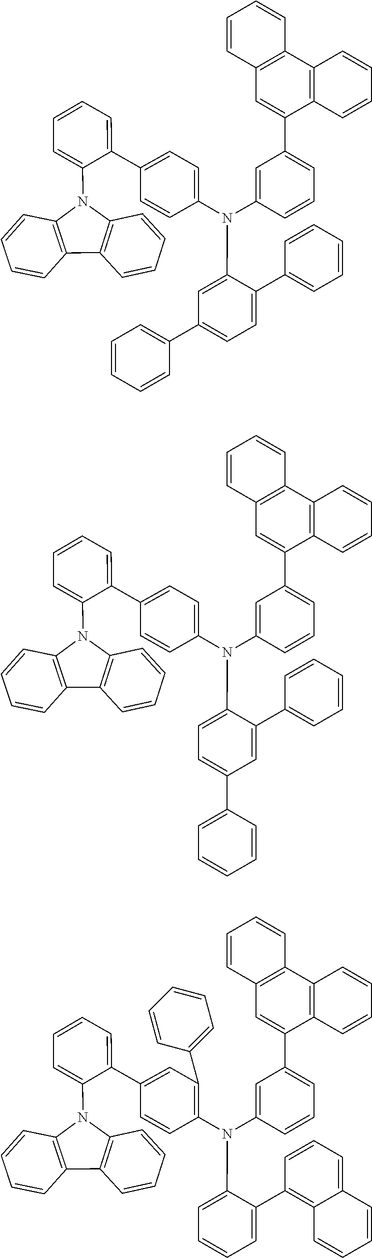 [SMILES: c1ccc(-c2ccc(-c3ccccc3)c(N(c3ccc(-c4ccccc4-n4c5ccccc5c5ccccc54)cc3)c3cccc(-c4cc5ccccc5c5ccccc45)c3)c2)cc1 ; c1ccc(-c2ccc(N(c3ccc(-c4ccccc4-n4c5ccccc5c5ccccc54)cc3)c3cccc(-c4cc5ccccc5c5ccccc45)c3)c(-c3ccccc3)c2)cc1 ; c1ccc(-c2cc(-c3ccccc3-n3c4ccccc4c4ccccc43)ccc2N(c2cccc(-c3cc4ccccc4c4ccccc34)c2)c2ccccc2-c2cccc3ccccc23)cc1] 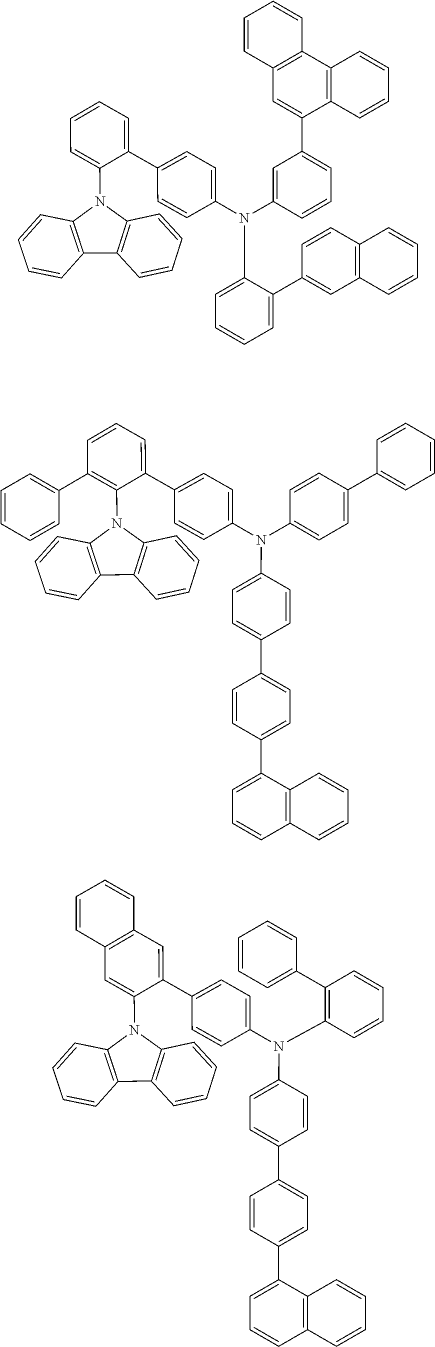 [SMILES: c1cc(-c2cc3ccccc3c3ccccc23)cc(N(c2ccc(-c3ccccc3-n3c4ccccc4c4ccccc43)cc2)c2ccccc2-c2ccc3ccccc3c2)c1 ; c1ccc(-c2ccc(N(c3ccc(-c4ccc(-c5cccc6ccccc56)cc4)cc3)c3ccc(-c4cccc(-c5ccccc5)c4-n4c5ccccc5c5ccccc54)cc3)cc2)cc1 ; c1ccc(-c2ccccc2N(c2ccc(-c3ccc(-c4cccc5ccccc45)cc3)cc2)c2ccc(-c3cc4ccccc4cc3-n3c4ccccc4c4ccccc43)cc2)cc1] 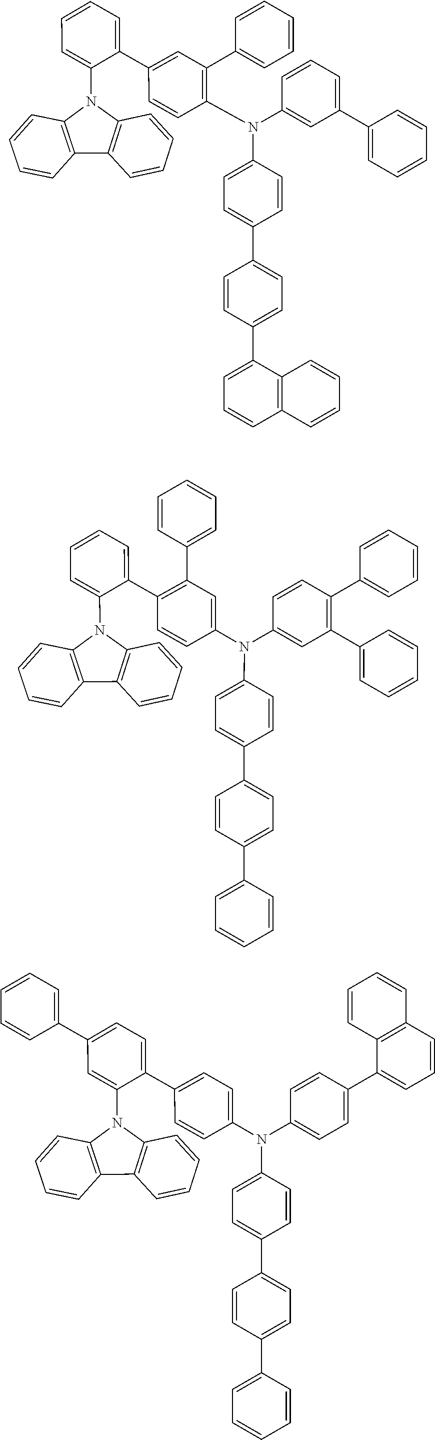 [SMILES: c1ccc(-c2cccc(N(c3ccc(-c4ccc(-c5cccc6ccccc56)cc4)cc3)c3ccc(-c4ccccc4-n4c5ccccc5c5ccccc54)cc3-c3ccccc3)c2)cc1 ; c1ccc(-c2ccc(-c3ccc(N(c4ccc(-c5ccccc5)c(-c5ccccc5)c4)c4ccc(-c5ccccc5-n5c6ccccc6c6ccccc65)c(-c5ccccc5)c4)cc3)cc2)cc1 ; c1ccc(-c2ccc(-c3ccc(N(c4ccc(-c5ccc(-c6ccccc6)cc5-n5c6ccccc6c6ccccc65)cc4)c4ccc(-c5cccc6ccccc56)cc4)cc3)cc2)cc1] 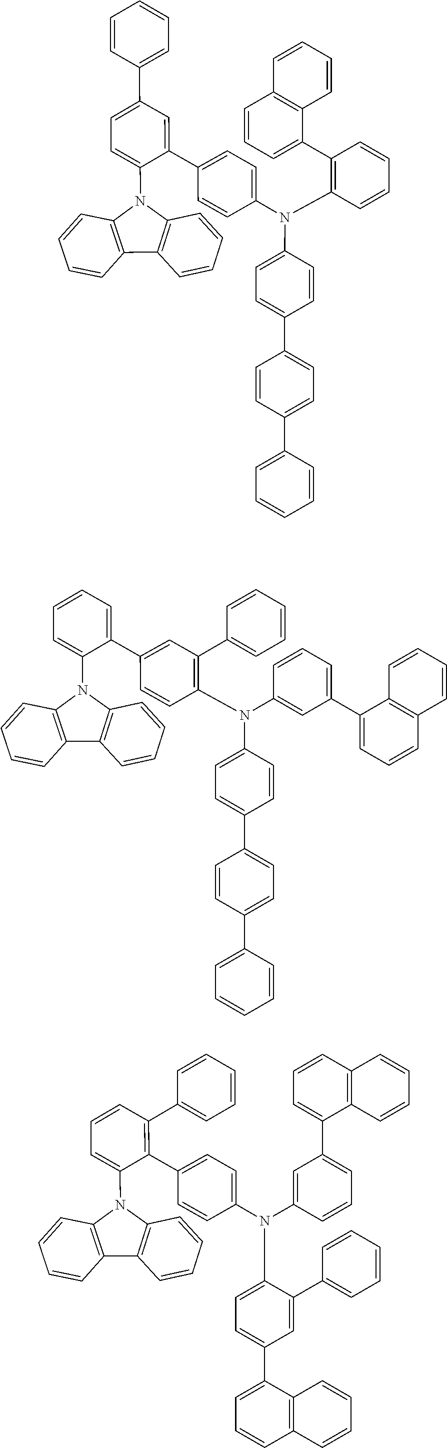 [SMILES: c1ccc(-c2ccc(-c3ccc(N(c4ccc(-c5cc(-c6ccccc6)ccc5-n5c6ccccc6c6ccccc65)cc4)c4ccccc4-c4cccc5ccccc45)cc3)cc2)cc1 ; c1ccc(-c2ccc(-c3ccc(N(c4cccc(-c5cccc6ccccc56)c4)c4ccc(-c5ccccc5-n5c6ccccc6c6ccccc65)cc4-c4ccccc4)cc3)cc2)cc1 ; c1ccc(-c2cc(-c3cccc4ccccc34)ccc2N(c2ccc(-c3c(-c4ccccc4)cccc3-n3c4ccccc4c4ccccc43)cc2)c2cccc(-c3cccc4ccccc34)c2)cc1] 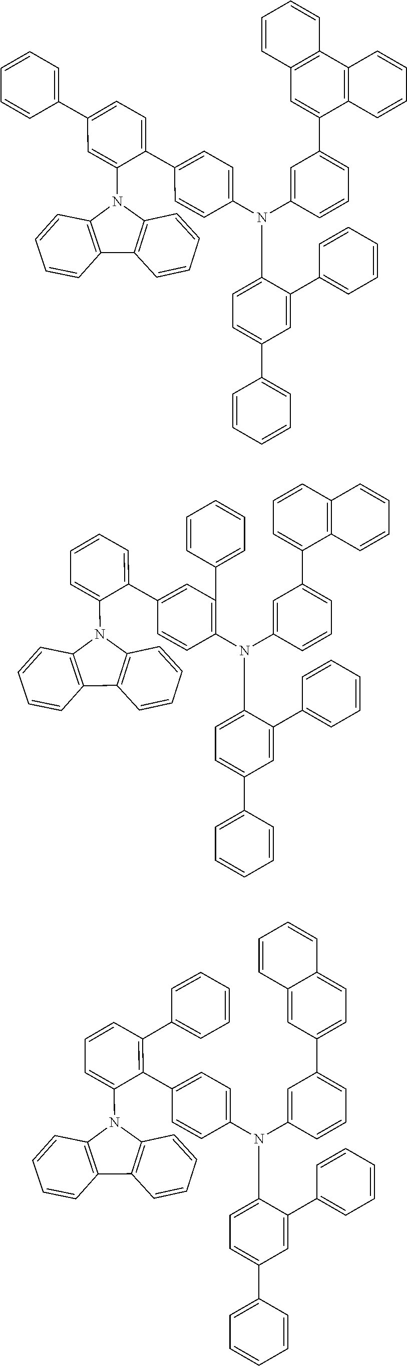 [SMILES: c1ccc(-c2ccc(N(c3ccc(-c4ccc(-c5ccccc5)cc4-n4c5ccccc5c5ccccc54)cc3)c3cccc(-c4cc5ccccc5c5ccccc45)c3)c(-c3ccccc3)c2)cc1 ; c1ccc(-c2ccc(N(c3cccc(-c4cccc5ccccc45)c3)c3ccc(-c4ccccc4-n4c5ccccc5c5ccccc54)cc3-c3ccccc3)c(-c3ccccc3)c2)cc1 ; c1ccc(-c2ccc(N(c3ccc(-c4c(-c5ccccc5)cccc4-n4c5ccccc5c5ccccc54)cc3)c3cccc(-c4ccc5ccccc5c4)c3)c(-c3ccccc3)c2)cc1] 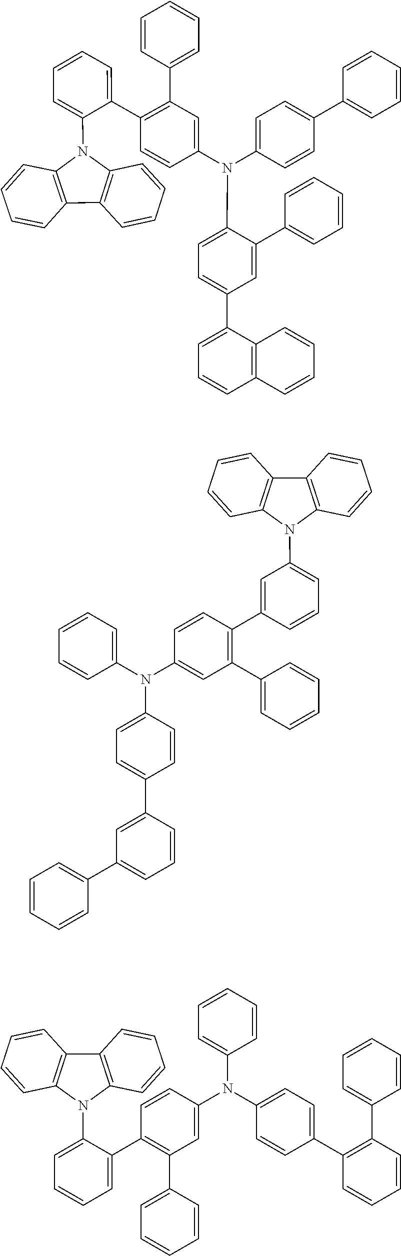 [SMILES: c1ccc(-c2ccc(N(c3ccc(-c4ccccc4-n4c5ccccc5c5ccccc54)c(-c4ccccc4)c3)c3ccc(-c4cccc5ccccc45)cc3-c3ccccc3)cc2)cc1 ; c1ccc(-c2cccc(-c3ccc(N(c4ccccc4)c4ccc(-c5cccc(-n6c7ccccc7c7ccccc76)c5)c(-c5ccccc5)c4)cc3)c2)cc1 ; c1ccc(-c2ccccc2-c2ccc(N(c3ccccc3)c3ccc(-c4ccccc4-n4c5ccccc5c5ccccc54)c(-c4ccccc4)c3)cc2)cc1] 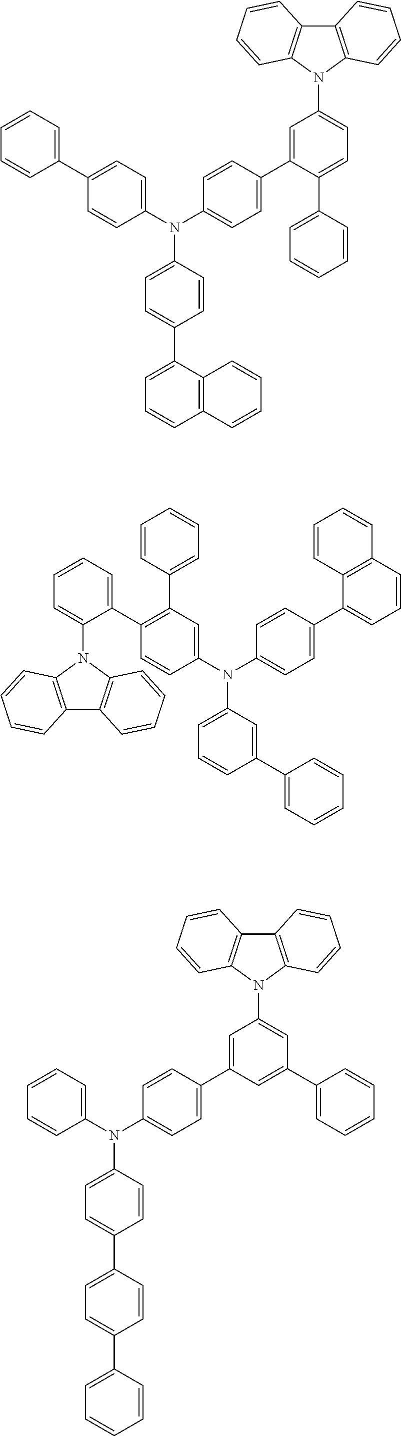 [SMILES: c1ccc(-c2ccc(N(c3ccc(-c4cc(-n5c6ccccc6c6ccccc65)ccc4-c4ccccc4)cc3)c3ccc(-c4cccc5ccccc45)cc3)cc2)cc1 ; c1ccc(-c2cccc(N(c3ccc(-c4cccc5ccccc45)cc3)c3ccc(-c4ccccc4-n4c5ccccc5c5ccccc54)c(-c4ccccc4)c3)c2)cc1 ; c1ccc(-c2ccc(-c3ccc(N(c4ccccc4)c4ccc(-c5cc(-c6ccccc6)cc(-n6c7ccccc7c7ccccc76)c5)cc4)cc3)cc2)cc1] 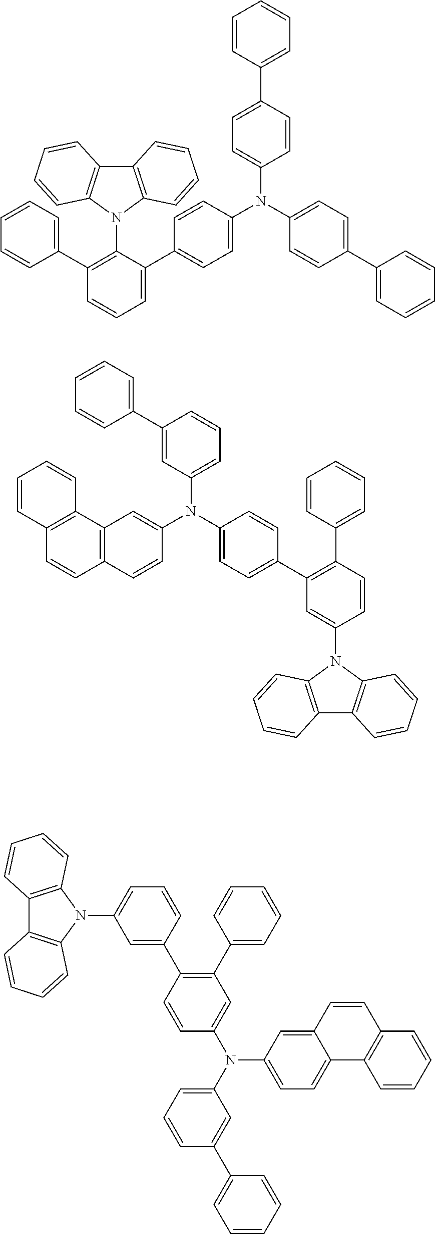 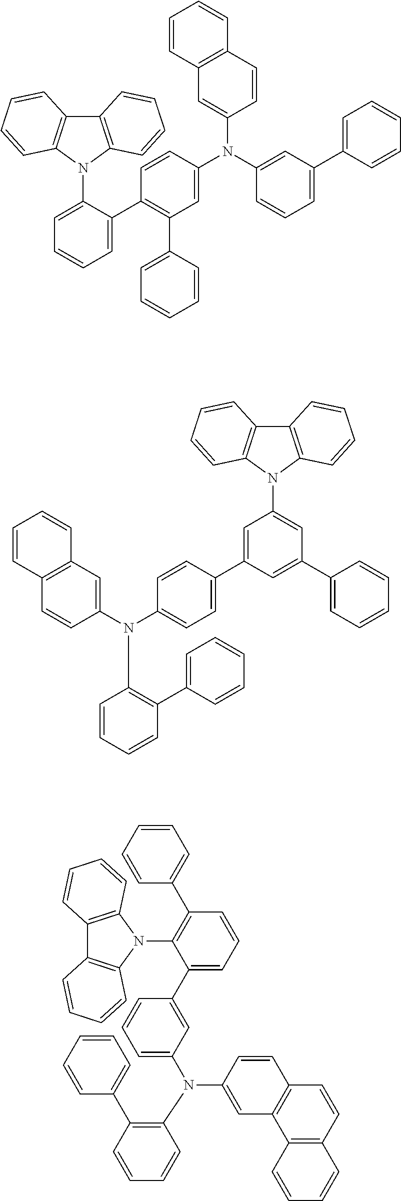 [SMILES: c1ccc(-c2cccc(N(c3ccc(-c4ccccc4-n4c5ccccc5c5ccccc54)c(-c4ccccc4)c3)c3ccc4ccccc4c3)c2)cc1 ; c1ccc(-c2cc(-c3ccc(N(c4ccc5ccccc5c4)c4ccccc4-c4ccccc4)cc3)cc(-n3c4ccccc4c4ccccc43)c2)cc1 ; c1ccc(-c2ccccc2N(c2cccc(-c3cccc(-c4ccccc4)c3-n3c4ccccc4c4ccccc43)c2)c2ccc3ccc4ccccc4c3c2)cc1] 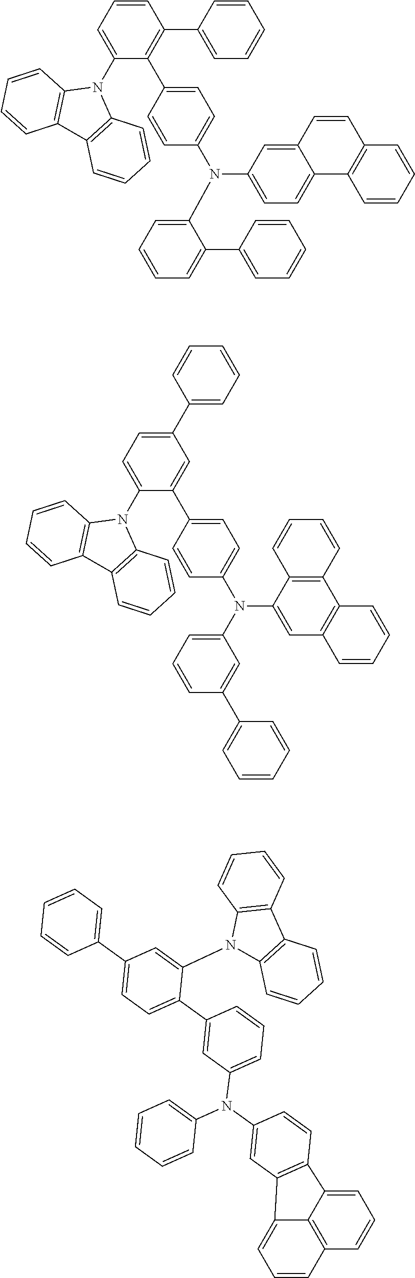 [SMILES: c1ccc(-c2ccccc2N(c2ccc(-c3c(-c4ccccc4)cccc3-n3c4ccccc4c4ccccc43)cc2)c2ccc3c(ccc4ccccc43)c2)cc1 ; c1ccc(-c2cccc(N(c3ccc(-c4cc(-c5ccccc5)ccc4-n4c5ccccc5c5ccccc54)cc3)c3cc4ccccc4c4ccccc34)c2)cc1 ; c1ccc(-c2ccc(-c3cccc(N(c4ccccc4)c4ccc5c(c4)-c4cccc6cccc-5c46)c3)c(-n3c4ccccc4c4ccccc43)c2)cc1] 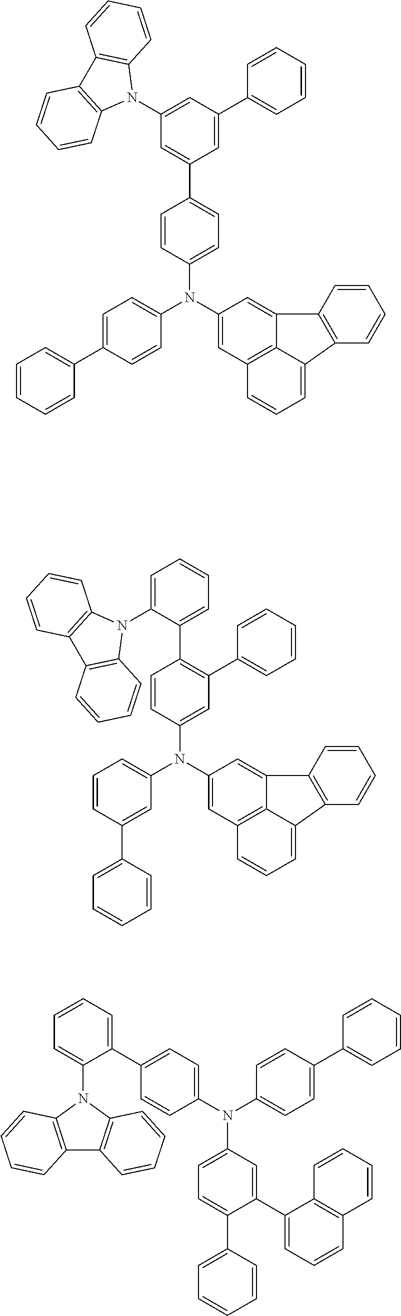 [SMILES: c1ccc(-c2ccc(N(c3ccc(-c4cc(-c5ccccc5)cc(-n5c6ccccc6c6ccccc65)c4)cc3)c3cc4c5c(cccc5c3)-c3ccccc3-4)cc2)cc1 ; c1ccc(-c2cccc(N(c3ccc(-c4ccccc4-n4c5ccccc5c5ccccc54)c(-c4ccccc4)c3)c3cc4c5c(cccc5c3)-c3ccccc3-4)c2)cc1 ; c1ccc(-c2ccc(N(c3ccc(-c4ccccc4-n4c5ccccc5c5ccccc54)cc3)c3ccc(-c4ccccc4)c(-c4cccc5ccccc45)c3)cc2)cc1] 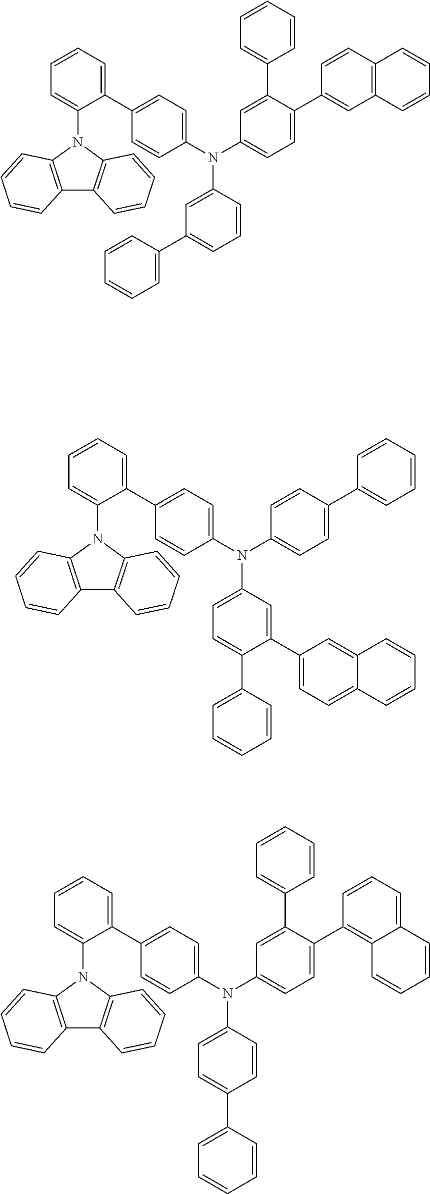 [SMILES: c1ccc(-c2cccc(N(c3ccc(-c4ccccc4-n4c5ccccc5c5ccccc54)cc3)c3ccc(-c4ccc5ccccc5c4)c(-c4ccccc4)c3)c2)cc1 ; c1ccc(-c2ccc(N(c3ccc(-c4ccccc4-n4c5ccccc5c5ccccc54)cc3)c3ccc(-c4ccccc4)c(-c4ccc5ccccc5c4)c3)cc2)cc1 ; c1ccc(-c2ccc(N(c3ccc(-c4ccccc4-n4c5ccccc5c5ccccc54)cc3)c3ccc(-c4cccc5ccccc45)c(-c4ccccc4)c3)cc2)cc1] 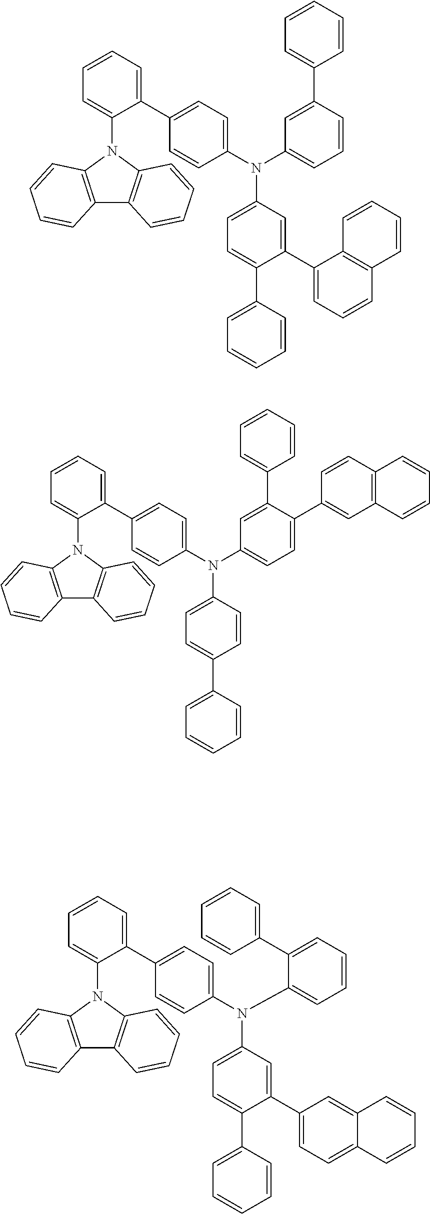 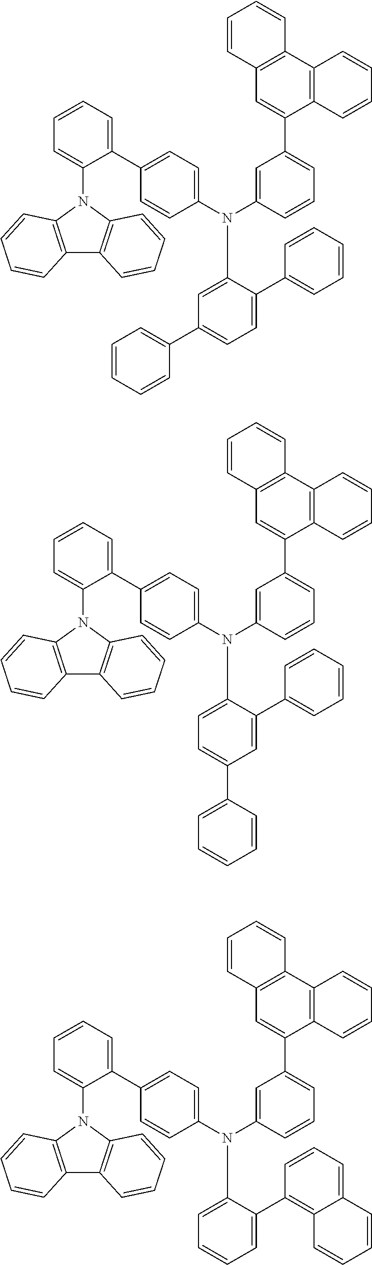 [SMILES: c1ccc(-c2ccc(-c3ccccc3)c(N(c3ccc(-c4ccccc4-n4c5ccccc5c5ccccc54)cc3)c3cccc(-c4cc5ccccc5c5ccccc45)c3)c2)cc1 ; c1ccc(-c2ccc(N(c3ccc(-c4ccccc4-n4c5ccccc5c5ccccc54)cc3)c3cccc(-c4cc5ccccc5c5ccccc45)c3)c(-c3ccccc3)c2)cc1 ; c1cc(-c2cc3ccccc3c3ccccc23)cc(N(c2ccc(-c3ccccc3-n3c4ccccc4c4ccccc43)cc2)c2ccccc2-c2cccc3ccccc23)c1] 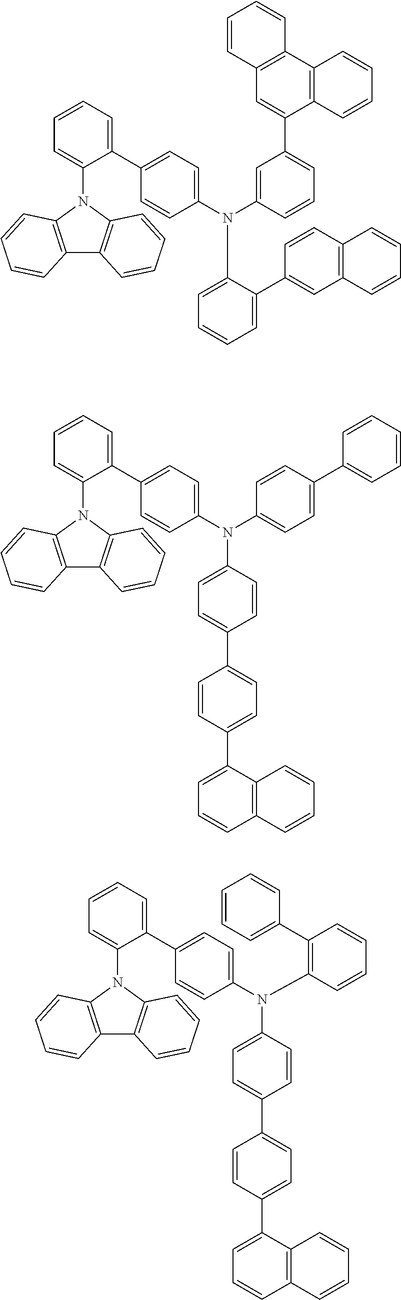 [SMILES: c1cc(-c2cc3ccccc3c3ccccc23)cc(N(c2ccc(-c3ccccc3-n3c4ccccc4c4ccccc43)cc2)c2ccccc2-c2ccc3ccccc3c2)c1 ; c1ccc(-c2ccc(N(c3ccc(-c4ccc(-c5cccc6ccccc56)cc4)cc3)c3ccc(-c4ccccc4-n4c5ccccc5c5ccccc54)cc3)cc2)cc1 ; c1ccc(-c2ccccc2N(c2ccc(-c3ccc(-c4cccc5ccccc45)cc3)cc2)c2ccc(-c3ccccc3-n3c4ccccc4c4ccccc43)cc2)cc1] 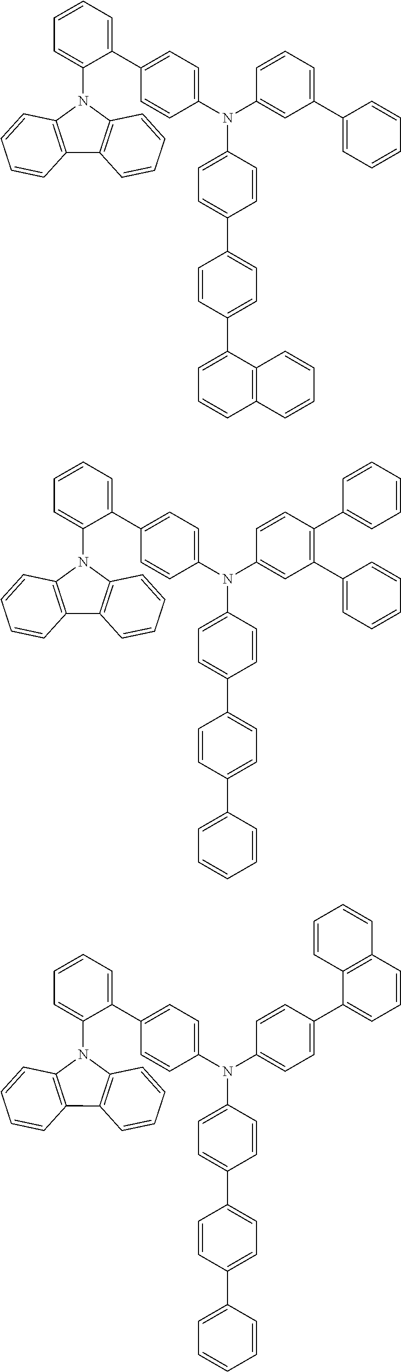 [SMILES: c1ccc(-c2cccc(N(c3ccc(-c4ccc(-c5cccc6ccccc56)cc4)cc3)c3ccc(-c4ccccc4-n4c5ccccc5c5ccccc54)cc3)c2)cc1 ; c1ccc(-c2ccc(-c3ccc(N(c4ccc(-c5ccccc5-n5c6ccccc6c6ccccc65)cc4)c4ccc(-c5ccccc5)c(-c5ccccc5)c4)cc3)cc2)cc1 ; c1ccc(-c2ccc(-c3ccc(N(c4ccc(-c5ccccc5-n5c6ccccc6c6ccccc65)cc4)c4ccc(-c5cccc6ccccc56)cc4)cc3)cc2)cc1] 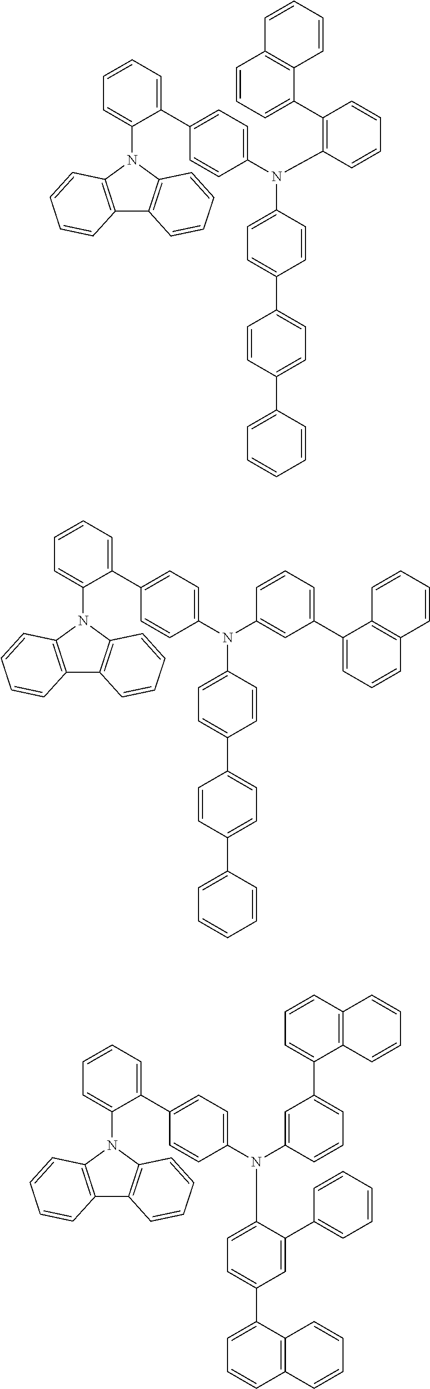 [SMILES: c1ccc(-c2ccc(-c3ccc(N(c4ccc(-c5ccccc5-n5c6ccccc6c6ccccc65)cc4)c4ccccc4-c4cccc5ccccc45)cc3)cc2)cc1 ; c1ccc(-c2ccc(-c3ccc(N(c4ccc(-c5ccccc5-n5c6ccccc6c6ccccc65)cc4)c4cccc(-c5cccc6ccccc56)c4)cc3)cc2)cc1 ; c1ccc(-c2cc(-c3cccc4ccccc34)ccc2N(c2ccc(-c3ccccc3-n3c4ccccc4c4ccccc43)cc2)c2cccc(-c3cccc4ccccc34)c2)cc1] 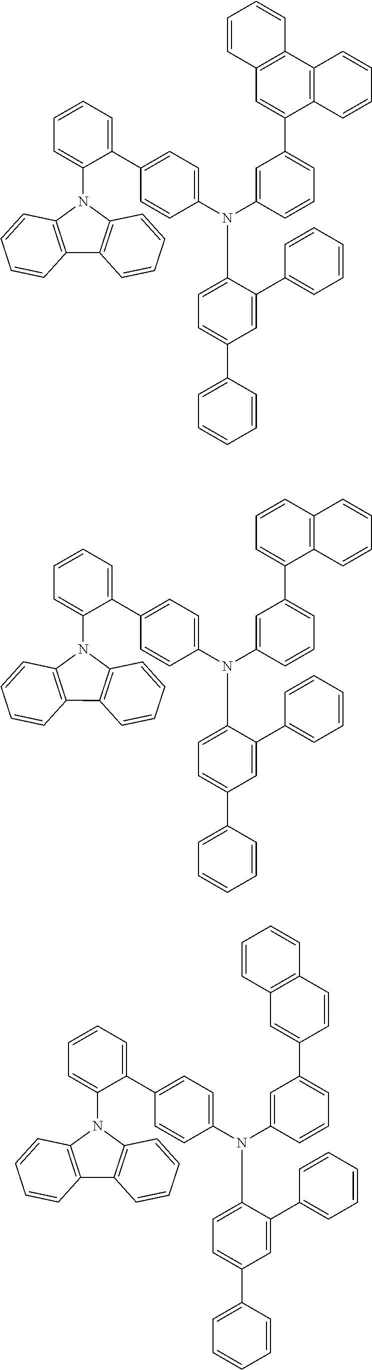 [SMILES: c1ccc(-c2ccc(N(c3ccc(-c4ccccc4-n4c5ccccc5c5ccccc54)cc3)c3cccc(-c4cc5ccccc5c5ccccc45)c3)c(-c3ccccc3)c2)cc1 ; c1ccc(-c2ccc(N(c3ccc(-c4ccccc4-n4c5ccccc5c5ccccc54)cc3)c3cccc(-c4cccc5ccccc45)c3)c(-c3ccccc3)c2)cc1 ; c1ccc(-c2ccc(N(c3ccc(-c4ccccc4-n4c5ccccc5c5ccccc54)cc3)c3cccc(-c4ccc5ccccc5c4)c3)c(-c3ccccc3)c2)cc1] 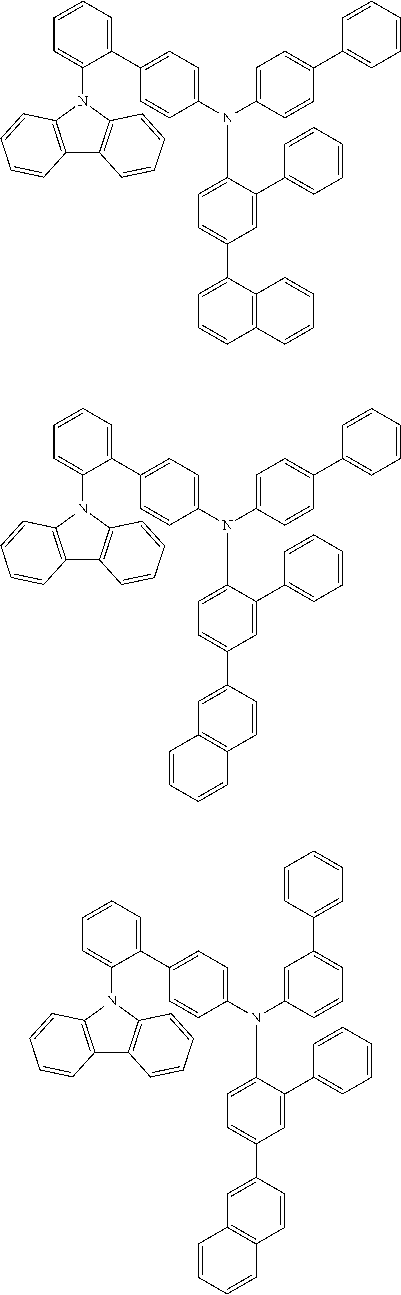 [SMILES: c1ccc(-c2ccc(N(c3ccc(-c4ccccc4-n4c5ccccc5c5ccccc54)cc3)c3ccc(-c4cccc5ccccc45)cc3-c3ccccc3)cc2)cc1 ; c1ccc(-c2ccc(N(c3ccc(-c4ccccc4-n4c5ccccc5c5ccccc54)cc3)c3ccc(-c4ccc5ccccc5c4)cc3-c3ccccc3)cc2)cc1 ; c1ccc(-c2cccc(N(c3ccc(-c4ccccc4-n4c5ccccc5c5ccccc54)cc3)c3ccc(-c4ccc5ccccc5c4)cc3-c3ccccc3)c2)cc1] 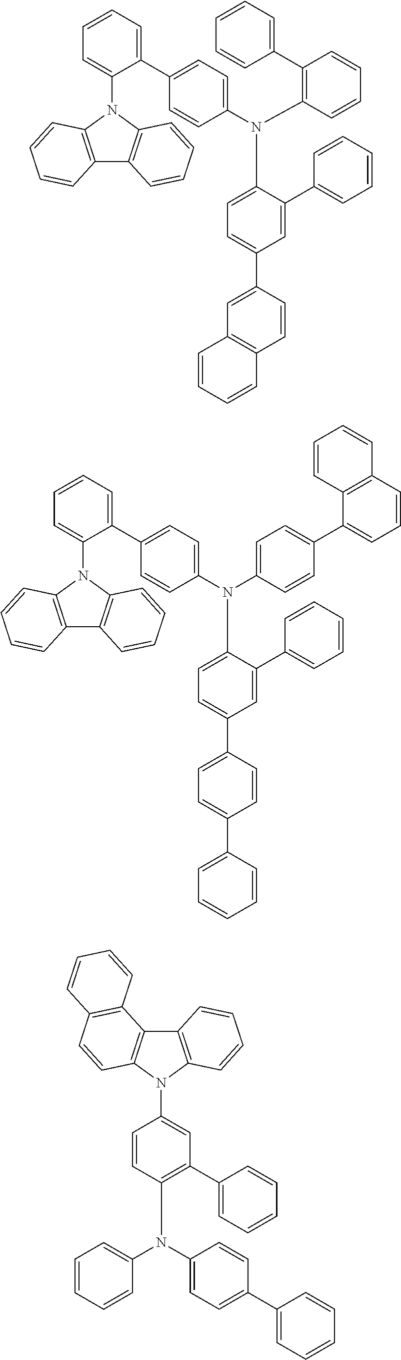 [SMILES: c1ccc(-c2ccccc2N(c2ccc(-c3ccccc3-n3c4ccccc4c4ccccc43)cc2)c2ccc(-c3ccc4ccccc4c3)cc2-c2ccccc2)cc1 ; c1ccc(-c2ccc(-c3ccc(N(c4ccc(-c5ccccc5-n5c6ccccc6c6ccccc65)cc4)c4ccc(-c5cccc6ccccc56)cc4)c(-c4ccccc4)c3)cc2)cc1 ; c1ccc(-c2ccc(N(c3ccccc3)c3ccc(-n4c5ccccc5c5c6ccccc6ccc54)cc3-c3ccccc3)cc2)cc1] 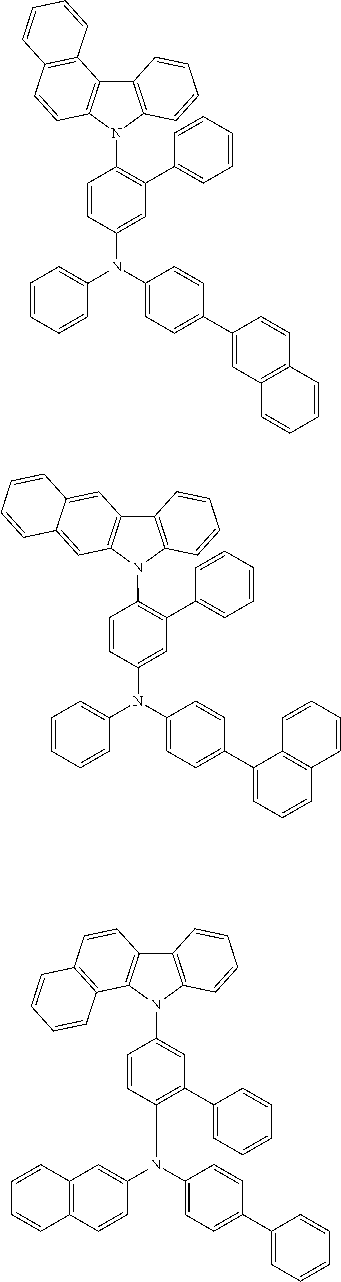 [SMILES: c1ccc(-c2cc(N(c3ccccc3)c3ccc(-c4ccc5ccccc5c4)cc3)ccc2-n2c3ccccc3c3c4ccccc4ccc32)cc1 ; c1ccc(-c2cc(N(c3ccccc3)c3ccc(-c4cccc5ccccc45)cc3)ccc2-n2c3ccccc3c3cc4ccccc4cc32)cc1 ; c1ccc(-c2ccc(N(c3ccc4ccccc4c3)c3ccc(-n4c5ccccc5c5ccc6ccccc6c54)cc3-c3ccccc3)cc2)cc1] 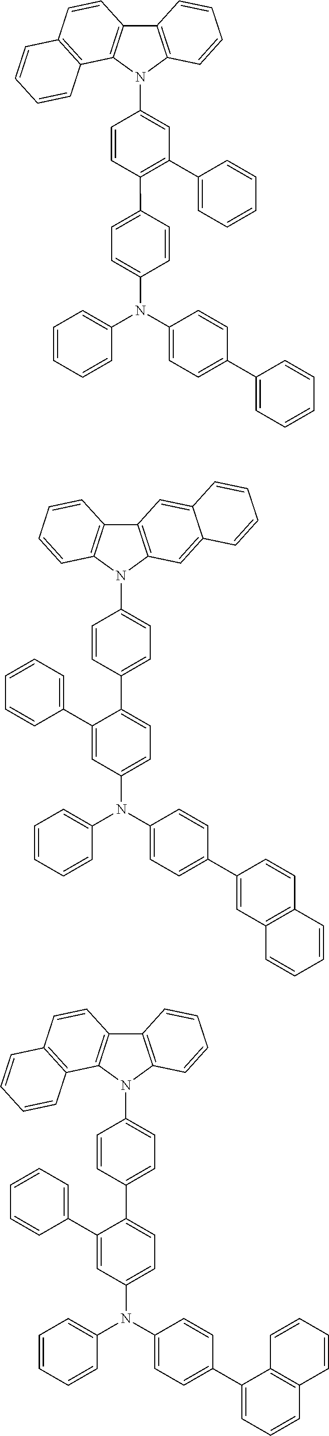 [SMILES: c1ccc(-c2ccc(N(c3ccccc3)c3ccc(-c4ccc(-n5c6ccccc6c6ccc7ccccc7c65)cc4-c4ccccc4)cc3)cc2)cc1 ; c1ccc(-c2cc(N(c3ccccc3)c3ccc(-c4ccc5ccccc5c4)cc3)ccc2-c2ccc(-n3c4ccccc4c4cc5ccccc5cc43)cc2)cc1 ; c1ccc(-c2cc(N(c3ccccc3)c3ccc(-c4cccc5ccccc45)cc3)ccc2-c2ccc(-n3c4ccccc4c4ccc5ccccc5c43)cc2)cc1] 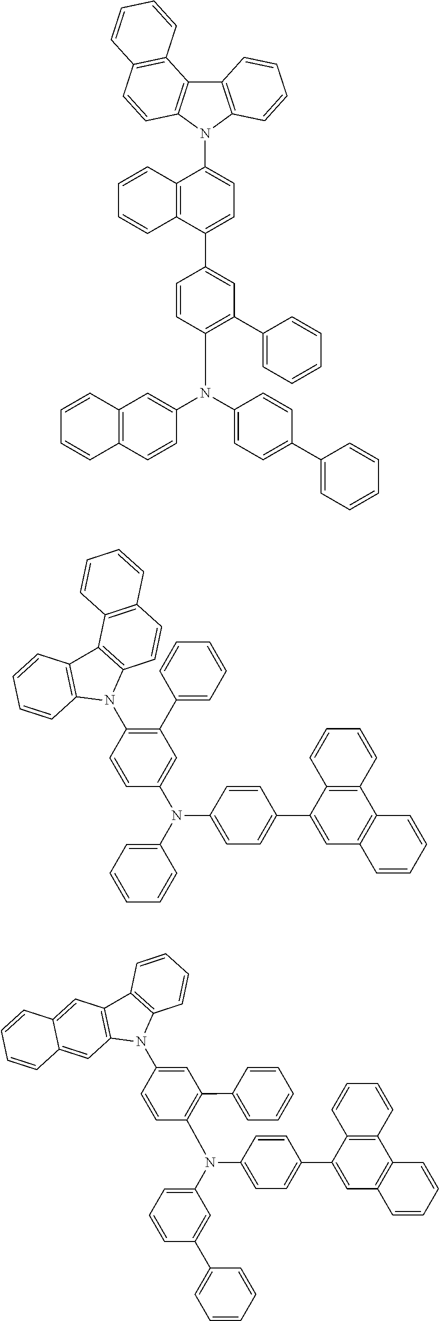 [SMILES: c1ccc(-c2ccc(N(c3ccc4ccccc4c3)c3ccc(-c4ccc(-n5c6ccccc6c6c7ccccc7ccc65)c5ccccc45)cc3-c3ccccc3)cc2)cc1 ; c1ccc(-c2cc(N(c3ccccc3)c3ccc(-c4cc5ccccc5c5ccccc45)cc3)ccc2-n2c3ccccc3c3c4ccccc4ccc32)cc1 ; c1ccc(-c2cccc(N(c3ccc(-c4cc5ccccc5c5ccccc45)cc3)c3ccc(-n4c5ccccc5c5cc6ccccc6cc54)cc3-c3ccccc3)c2)cc1] 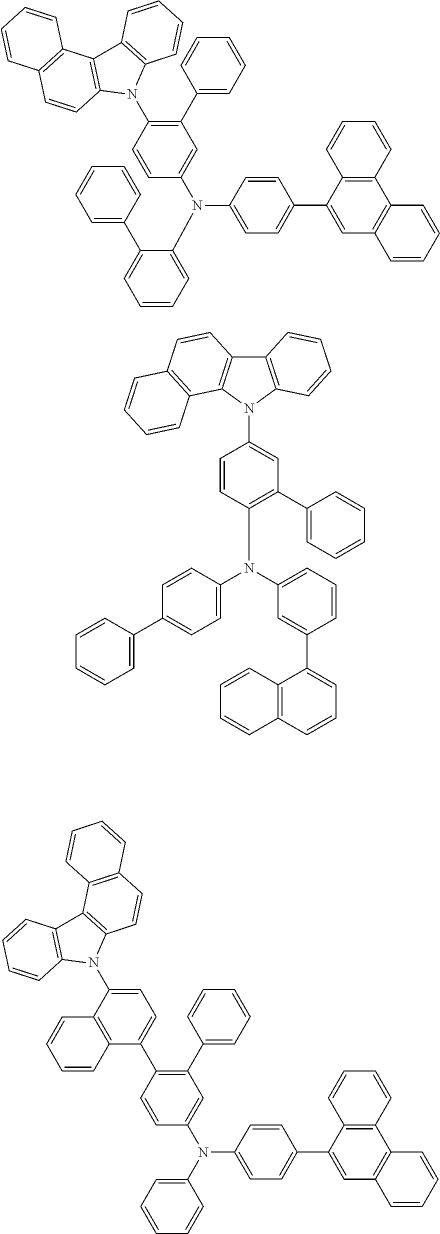 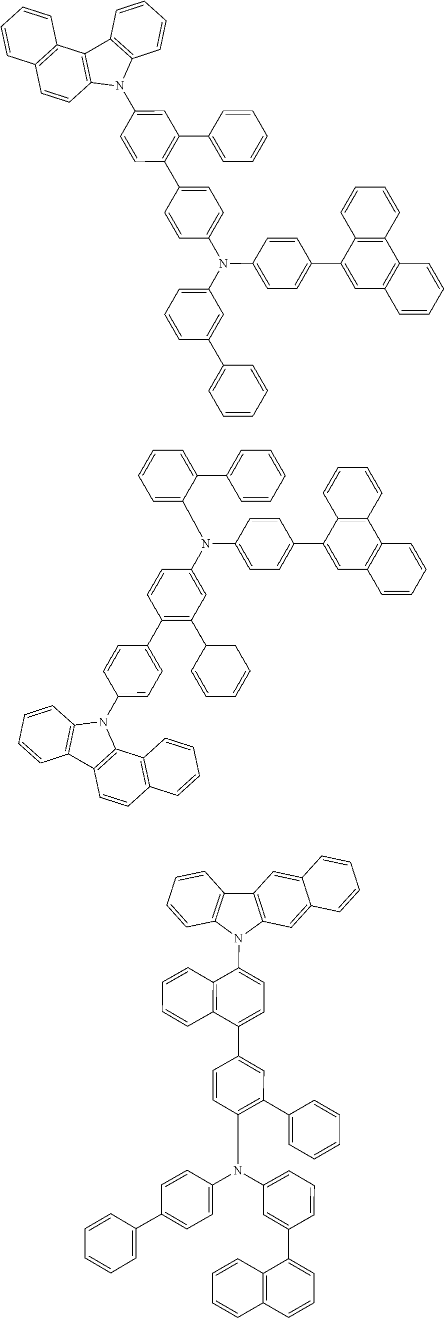 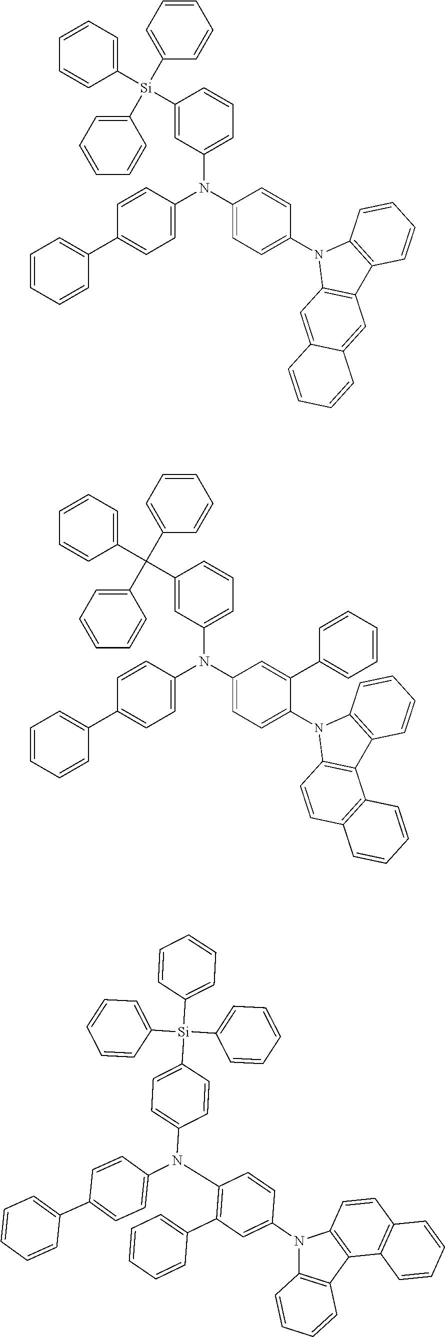 [SMILES: c1ccc(-c2ccc(N(c3ccc(-n4c5ccccc5c5cc6ccccc6cc54)cc3)c3cccc([Si](c4ccccc4)(c4ccccc4)c4ccccc4)c3)cc2)cc1 ; c1ccc(-c2ccc(N(c3cccc(C(c4ccccc4)(c4ccccc4)c4ccccc4)c3)c3ccc(-n4c5ccccc5c5c6ccccc6ccc54)c(-c4ccccc4)c3)cc2)cc1 ; c1ccc(-c2ccc(N(c3ccc([Si](c4ccccc4)(c4ccccc4)c4ccccc4)cc3)c3ccc(-n4c5ccccc5c5c6ccccc6ccc54)cc3-c3ccccc3)cc2)cc1] 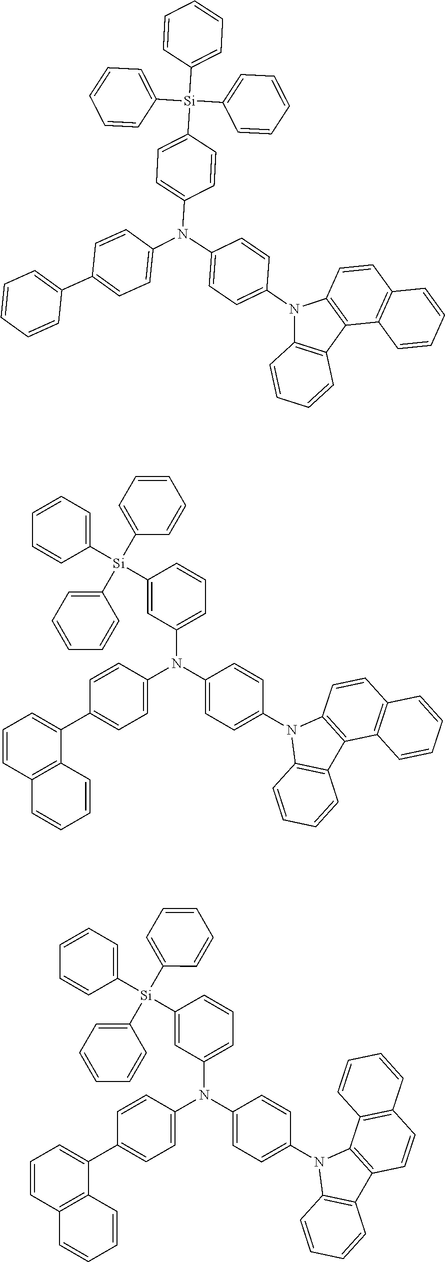 [SMILES: c1ccc(-c2ccc(N(c3ccc(-n4c5ccccc5c5c6ccccc6ccc54)cc3)c3ccc([Si](c4ccccc4)(c4ccccc4)c4ccccc4)cc3)cc2)cc1 ; c1ccc([Si](c2ccccc2)(c2ccccc2)c2cccc(N(c3ccc(-c4cccc5ccccc45)cc3)c3ccc(-n4c5ccccc5c5c6ccccc6ccc54)cc3)c2)cc1 ; c1ccc([Si](c2ccccc2)(c2ccccc2)c2cccc(N(c3ccc(-c4cccc5ccccc45)cc3)c3ccc(-n4c5ccccc5c5ccc6ccccc6c54)cc3)c2)cc1] 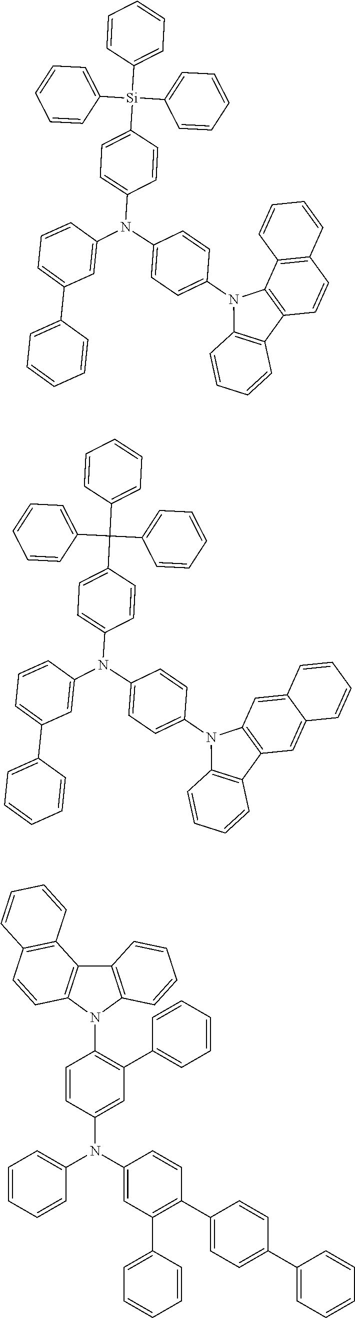 [SMILES: c1ccc(-c2cccc(N(c3ccc(-n4c5ccccc5c5ccc6ccccc6c54)cc3)c3ccc([Si](c4ccccc4)(c4ccccc4)c4ccccc4)cc3)c2)cc1 ; c1ccc(-c2cccc(N(c3ccc(-n4c5ccccc5c5cc6ccccc6cc54)cc3)c3ccc(C(c4ccccc4)(c4ccccc4)c4ccccc4)cc3)c2)cc1 ; c1ccc(-c2ccc(-c3ccc(N(c4ccccc4)c4ccc(-n5c6ccccc6c6c7ccccc7ccc65)c(-c5ccccc5)c4)cc3-c3ccccc3)cc2)cc1] 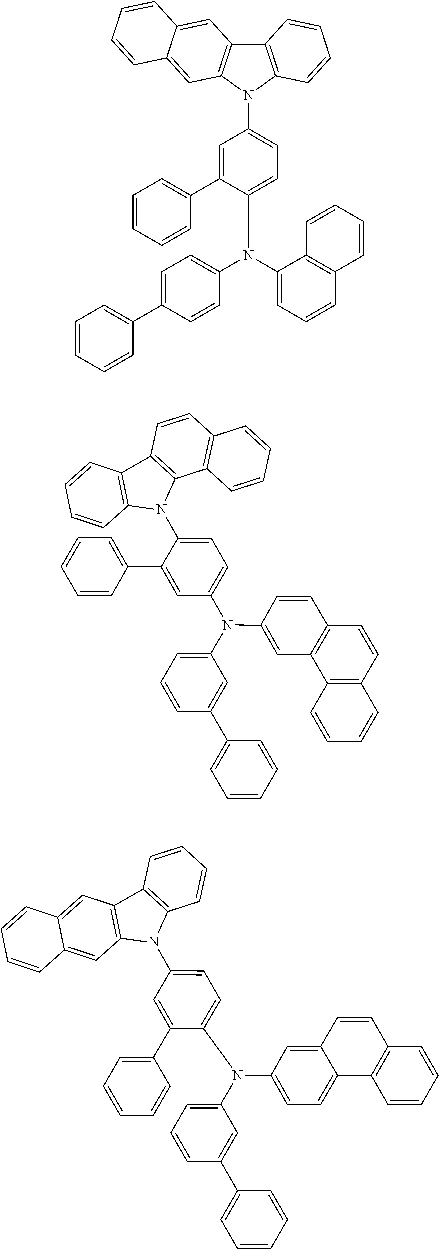 [SMILES: c1ccc(-c2ccc(N(c3ccc(-n4c5ccccc5c5cc6ccccc6cc54)cc3-c3ccccc3)c3cccc4ccccc34)cc2)cc1 ; c1ccc(-c2cccc(N(c3ccc(-n4c5ccccc5c5ccc6ccccc6c54)c(-c4ccccc4)c3)c3ccc4ccc5ccccc5c4c3)c2)cc1 ; c1ccc(-c2cccc(N(c3ccc4c(ccc5ccccc54)c3)c3ccc(-n4c5ccccc5c5cc6ccccc6cc54)cc3-c3ccccc3)c2)cc1] 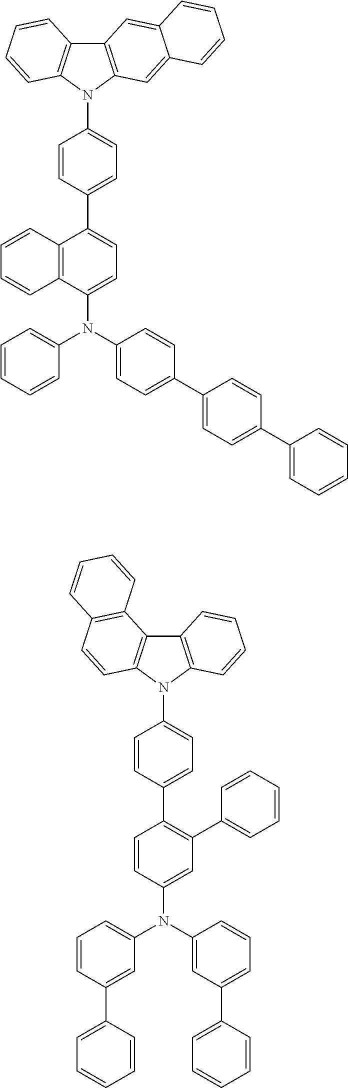 [SMILES: c1ccc(-c2ccc(-c3ccc(N(c4ccccc4)c4ccc(-c5ccc(-n6c7ccccc7c7cc8ccccc8cc76)cc5)c5ccccc45)cc3)cc2)cc1 ; c1ccc(-c2cccc(N(c3cccc(-c4ccccc4)c3)c3ccc(-c4ccc(-n5c6ccccc6c6c7ccccc7ccc65)cc4)c(-c4ccccc4)c3)c2)cc1] 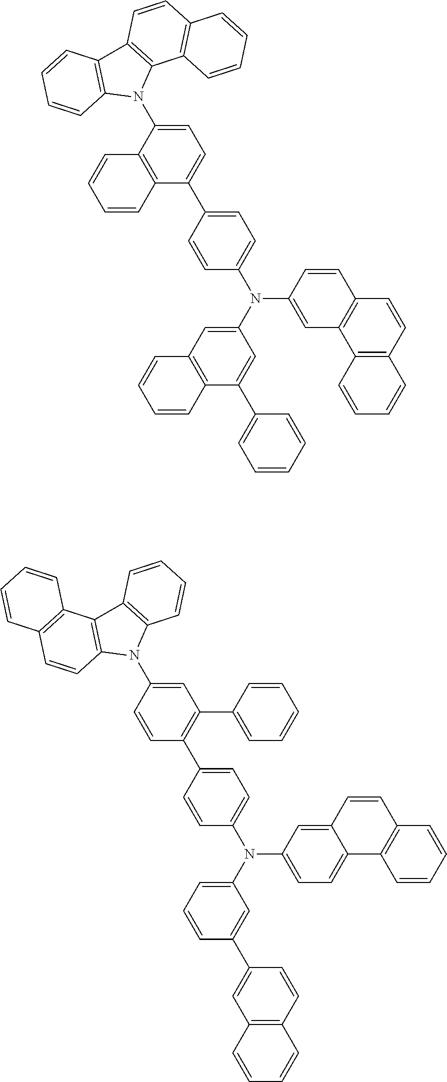 [SMILES: c1ccc(-c2cc(N(c3ccc(-c4ccc(-n5c6ccccc6c6ccc7ccccc7c65)c5ccccc45)cc3)c3ccc4ccc5ccccc5c4c3)cc3ccccc23)cc1 ; c1ccc(-c2cc(-n3c4ccccc4c4c5ccccc5ccc43)ccc2-c2ccc(N(c3cccc(-c4ccc5ccccc5c4)c3)c3ccc4c(ccc5ccccc54)c3)cc2)cc1] 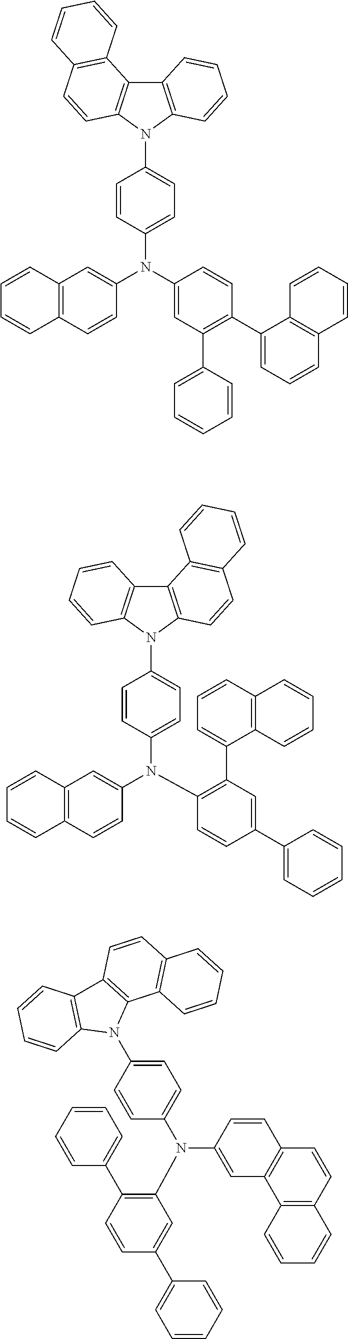 [SMILES: c1ccc(-c2cc(N(c3ccc(-n4c5ccccc5c5c6ccccc6ccc54)cc3)c3ccc4ccccc4c3)ccc2-c2cccc3ccccc23)cc1 ; c1ccc(-c2ccc(N(c3ccc(-n4c5ccccc5c5c6ccccc6ccc54)cc3)c3ccc4ccccc4c3)c(-c3cccc4ccccc34)c2)cc1 ; c1ccc(-c2ccc(-c3ccccc3)c(N(c3ccc(-n4c5ccccc5c5ccc6ccccc6c54)cc3)c3ccc4ccc5ccccc5c4c3)c2)cc1] 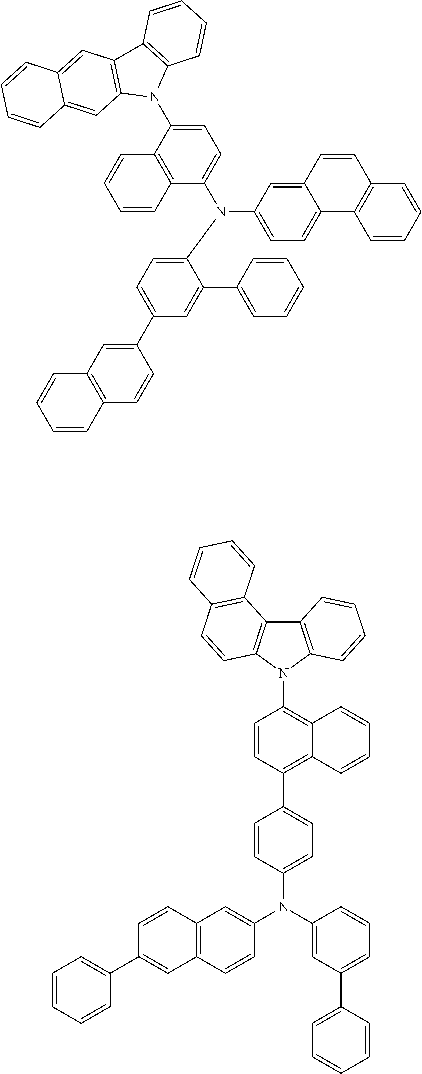 [SMILES: c1ccc(-c2cc(-c3ccc4ccccc4c3)ccc2N(c2ccc3c(ccc4ccccc43)c2)c2ccc(-n3c4ccccc4c4cc5ccccc5cc43)c3ccccc23)cc1 ; c1ccc(-c2cccc(N(c3ccc(-c4ccc(-n5c6ccccc6c6c7ccccc7ccc65)c5ccccc45)cc3)c3ccc4cc(-c5ccccc5)ccc4c3)c2)cc1] 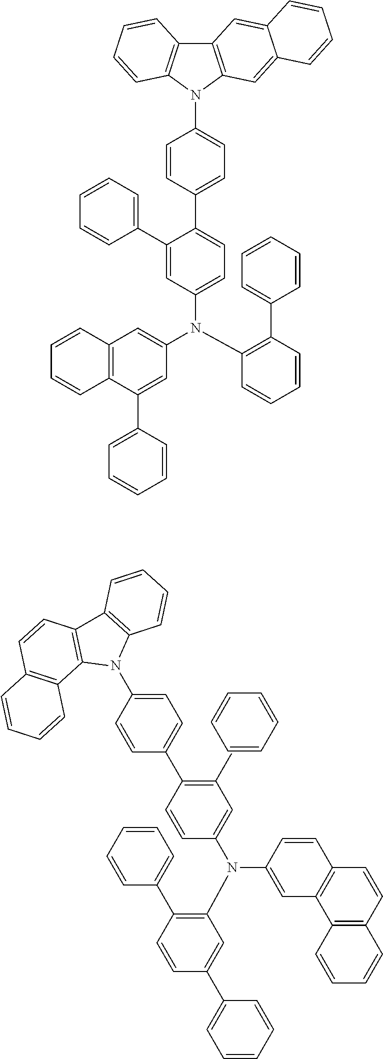 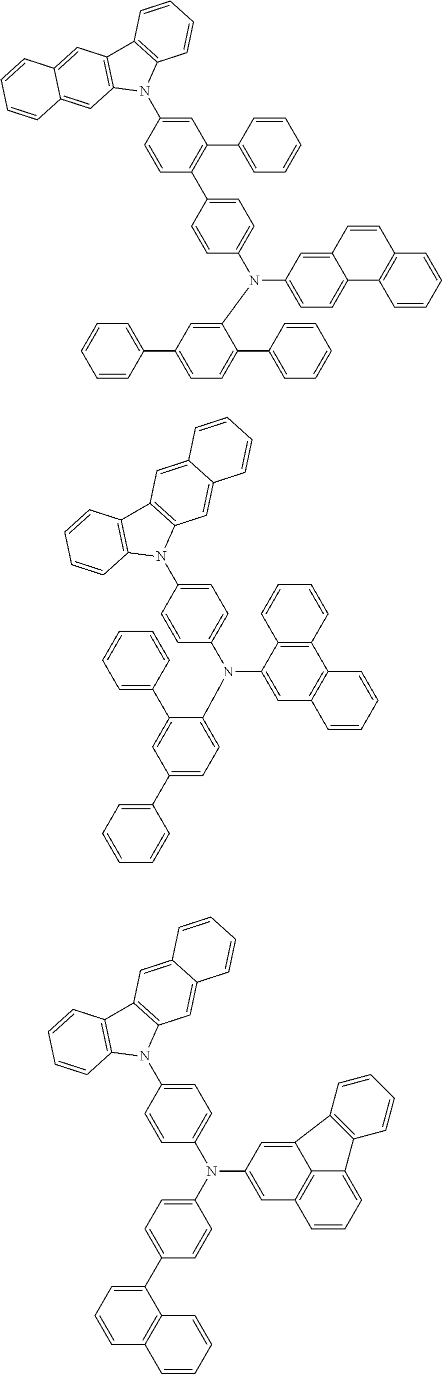 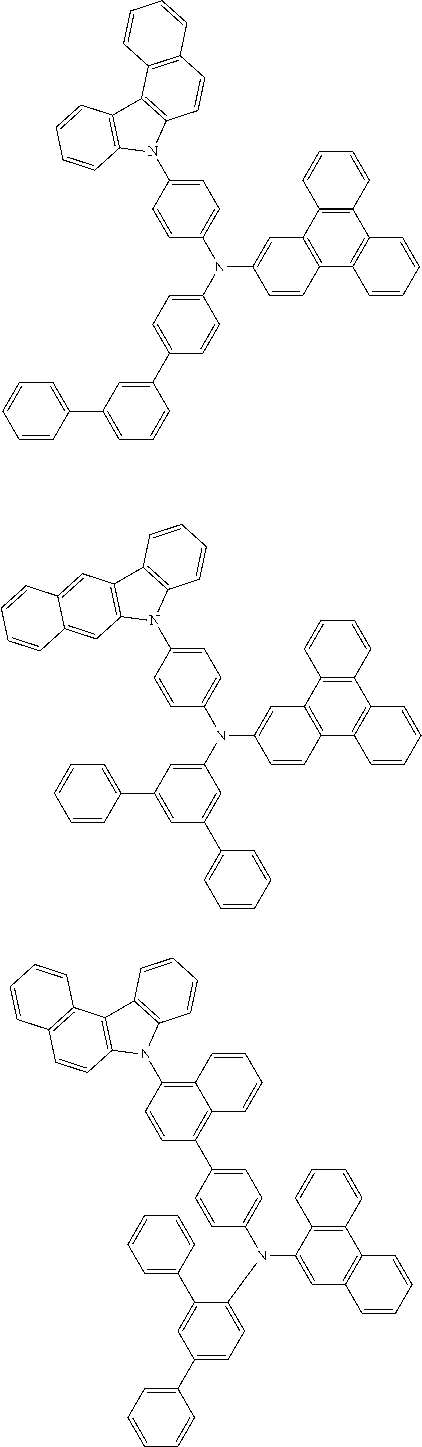 [SMILES: c1ccc(-c2cccc(-c3ccc(N(c4ccc(-n5c6ccccc6c6c7ccccc7ccc65)cc4)c4ccc5c6ccccc6c6ccccc6c5c4)cc3)c2)cc1 ; c1ccc(-c2cc(-c3ccccc3)cc(N(c3ccc(-n4c5ccccc5c5cc6ccccc6cc54)cc3)c3ccc4c5ccccc5c5ccccc5c4c3)c2)cc1 ; c1ccc(-c2ccc(N(c3ccc(-c4ccc(-n5c6ccccc6c6c7ccccc7ccc65)c5ccccc45)cc3)c3cc4ccccc4c4ccccc34)c(-c3ccccc3)c2)cc1] 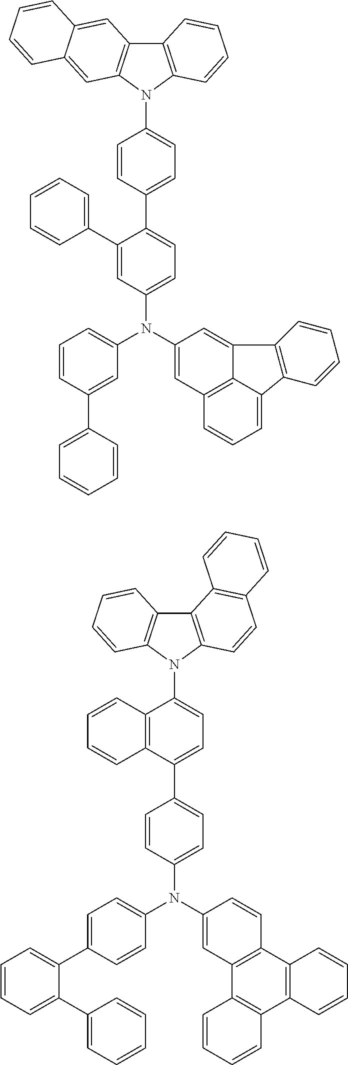 [SMILES: c1ccc(-c2cccc(N(c3ccc(-c4ccc(-n5c6ccccc6c6cc7ccccc7cc65)cc4)c(-c4ccccc4)c3)c3cc4c5c(cccc5c3)-c3ccccc3-4)c2)cc1 ; c1ccc(-c2ccccc2-c2ccc(N(c3ccc(-c4ccc(-n5c6ccccc6c6c7ccccc7ccc65)c5ccccc45)cc3)c3ccc4c5ccccc5c5ccccc5c4c3)cc2)cc1] 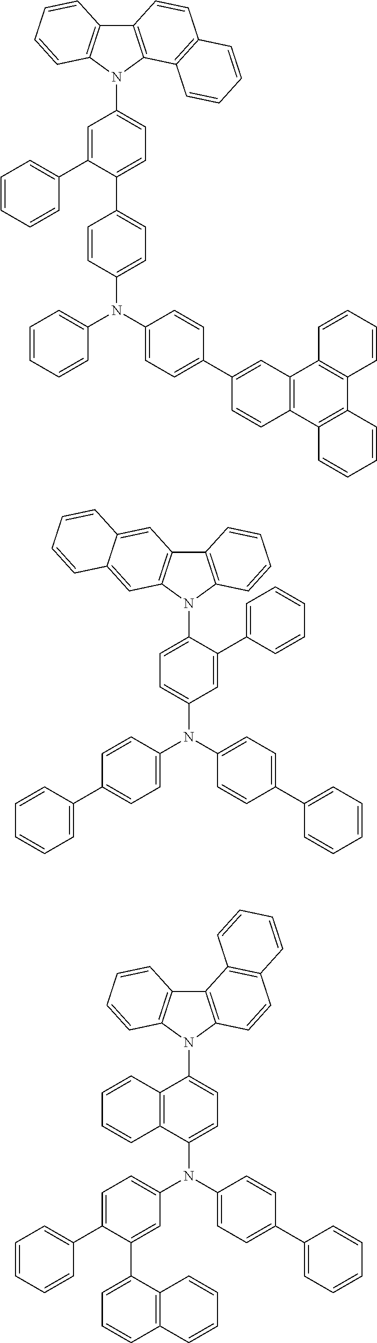 [SMILES: c1ccc(-c2cc(-n3c4ccccc4c4ccc5ccccc5c43)ccc2-c2ccc(N(c3ccccc3)c3ccc(-c4ccc5c6ccccc6c6ccccc6c5c4)cc3)cc2)cc1 ; c1ccc(-c2ccc(N(c3ccc(-c4ccccc4)cc3)c3ccc(-n4c5ccccc5c5cc6ccccc6cc54)c(-c4ccccc4)c3)cc2)cc1 ; c1ccc(-c2ccc(N(c3ccc(-c4ccccc4)c(-c4cccc5ccccc45)c3)c3ccc(-n4c5ccccc5c5c6ccccc6ccc54)c4ccccc34)cc2)cc1] 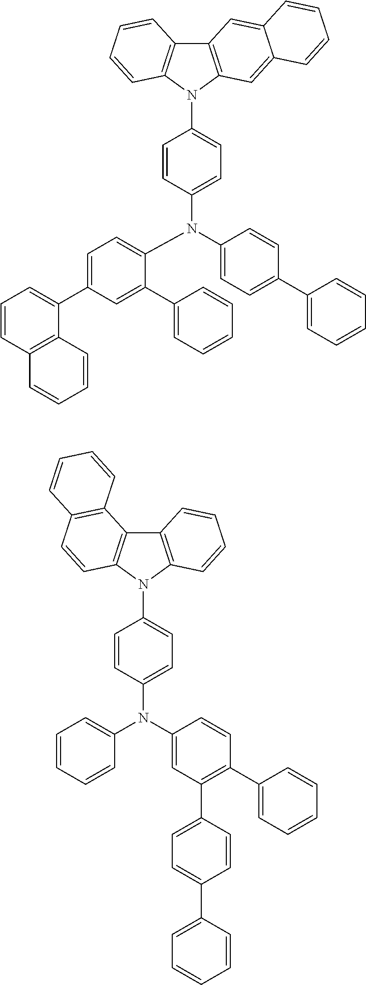 [SMILES: c1ccc(-c2ccc(N(c3ccc(-n4c5ccccc5c5cc6ccccc6cc54)cc3)c3ccc(-c4cccc5ccccc45)cc3-c3ccccc3)cc2)cc1 ; c1ccc(-c2ccc(-c3cc(N(c4ccccc4)c4ccc(-n5c6ccccc6c6c7ccccc7ccc65)cc4)ccc3-c3ccccc3)cc2)cc1] 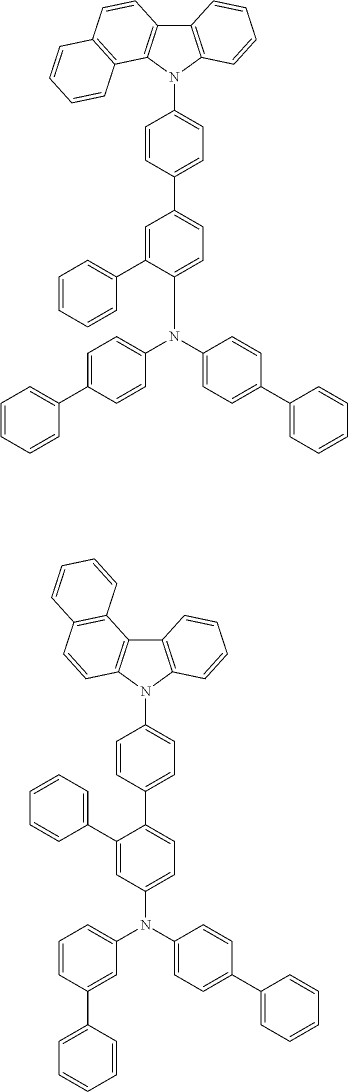 [SMILES: c1ccc(-c2ccc(N(c3ccc(-c4ccccc4)cc3)c3ccc(-c4ccc(-n5c6ccccc6c6ccc7ccccc7c65)cc4)cc3-c3ccccc3)cc2)cc1 ; c1ccc(-c2ccc(N(c3cccc(-c4ccccc4)c3)c3ccc(-c4ccc(-n5c6ccccc6c6c7ccccc7ccc65)cc4)c(-c4ccccc4)c3)cc2)cc1] 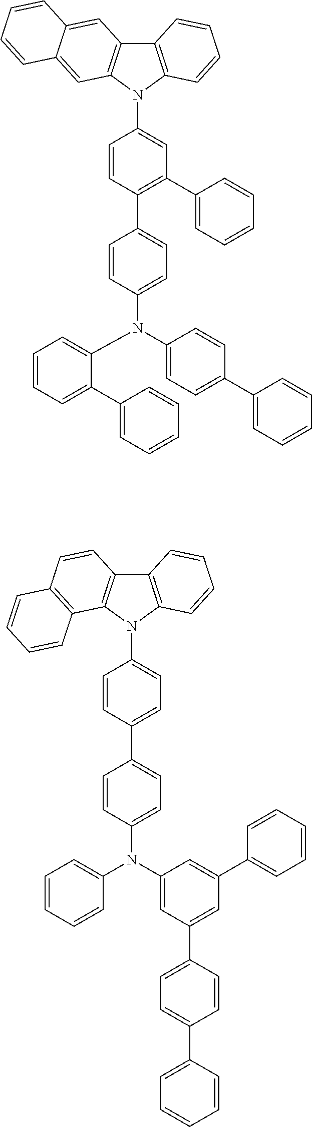 [SMILES: c1ccc(-c2ccc(N(c3ccc(-c4ccc(-n5c6ccccc6c6cc7ccccc7cc65)cc4-c4ccccc4)cc3)c3ccccc3-c3ccccc3)cc2)cc1 ; c1ccc(-c2ccc(-c3cc(-c4ccccc4)cc(N(c4ccccc4)c4ccc(-c5ccc(-n6c7ccccc7c7ccc8ccccc8c76)cc5)cc4)c3)cc2)cc1] 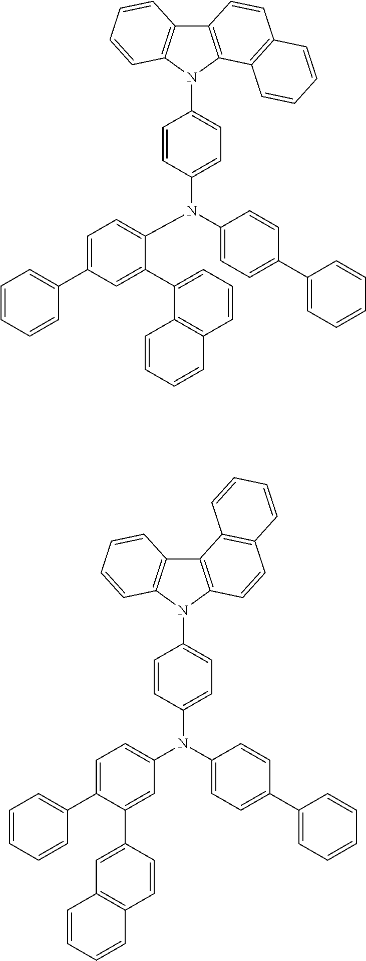 [SMILES: c1ccc(-c2ccc(N(c3ccc(-n4c5ccccc5c5ccc6ccccc6c54)cc3)c3ccc(-c4ccccc4)cc3-c3cccc4ccccc34)cc2)cc1 ; c1ccc(-c2ccc(N(c3ccc(-n4c5ccccc5c5c6ccccc6ccc54)cc3)c3ccc(-c4ccccc4)c(-c4ccc5ccccc5c4)c3)cc2)cc1] 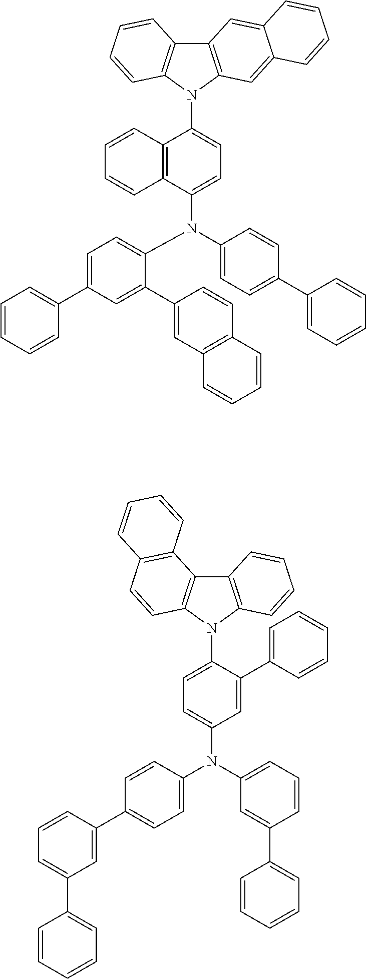 [SMILES: c1ccc(-c2ccc(N(c3ccc(-c4ccccc4)cc3-c3ccc4ccccc4c3)c3ccc(-n4c5ccccc5c5cc6ccccc6cc54)c4ccccc34)cc2)cc1 ; c1ccc(-c2cccc(-c3ccc(N(c4cccc(-c5ccccc5)c4)c4ccc(-n5c6ccccc6c6c7ccccc7ccc65)c(-c5ccccc5)c4)cc3)c2)cc1] 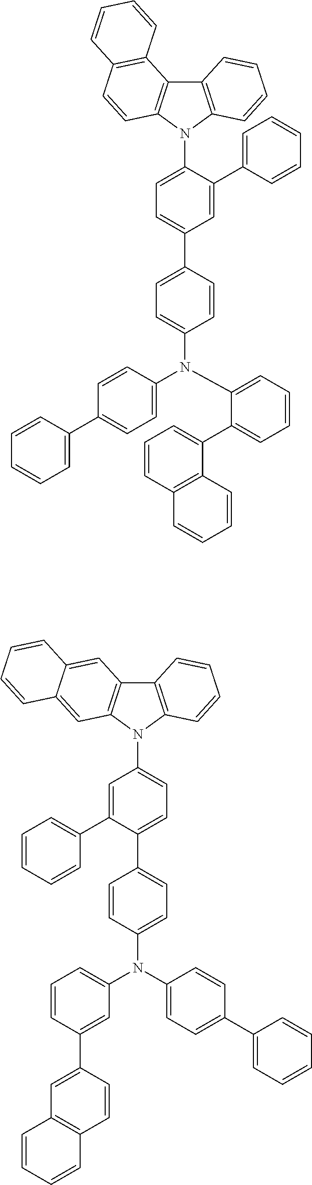 [SMILES: c1ccc(-c2ccc(N(c3ccc(-c4ccc(-n5c6ccccc6c6c7ccccc7ccc65)c(-c5ccccc5)c4)cc3)c3ccccc3-c3cccc4ccccc34)cc2)cc1 ; c1ccc(-c2ccc(N(c3ccc(-c4ccc(-n5c6ccccc6c6cc7ccccc7cc65)cc4-c4ccccc4)cc3)c3cccc(-c4ccc5ccccc5c4)c3)cc2)cc1] 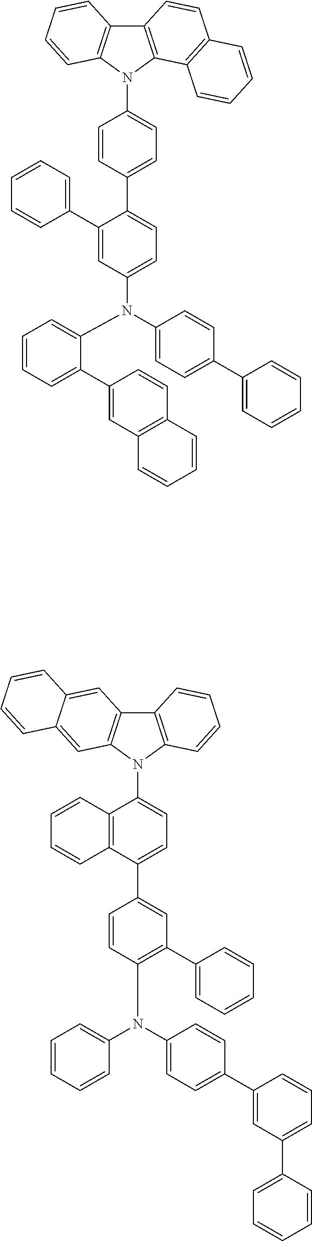 [SMILES: c1ccc(-c2ccc(N(c3ccc(-c4ccc(-n5c6ccccc6c6ccc7ccccc7c65)cc4)c(-c4ccccc4)c3)c3ccccc3-c3ccc4ccccc4c3)cc2)cc1 ; c1ccc(-c2cccc(-c3ccc(N(c4ccccc4)c4ccc(-c5ccc(-n6c7ccccc7c7cc8ccccc8cc76)c6ccccc56)cc4-c4ccccc4)cc3)c2)cc1] 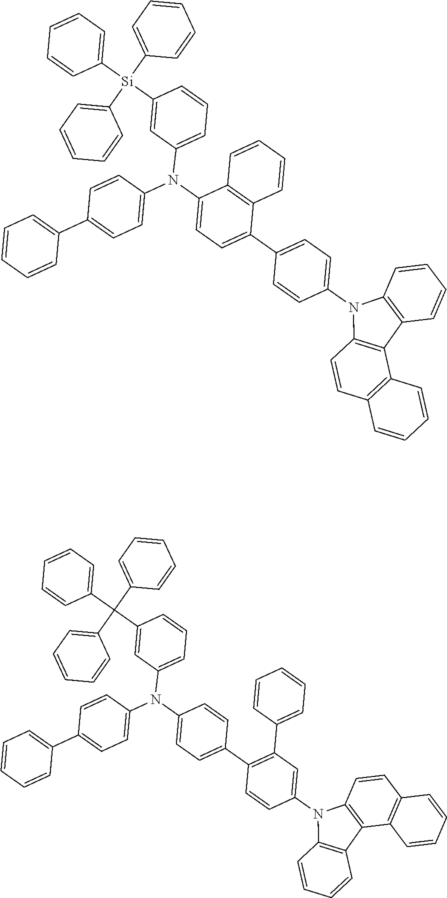 [SMILES: c1ccc(-c2ccc(N(c3cccc([Si](c4ccccc4)(c4ccccc4)c4ccccc4)c3)c3ccc(-c4ccc(-n5c6ccccc6c6c7ccccc7ccc65)cc4)c4ccccc34)cc2)cc1 ; c1ccc(-c2ccc(N(c3ccc(-c4ccc(-n5c6ccccc6c6c7ccccc7ccc65)cc4-c4ccccc4)cc3)c3cccc(C(c4ccccc4)(c4ccccc4)c4ccccc4)c3)cc2)cc1] 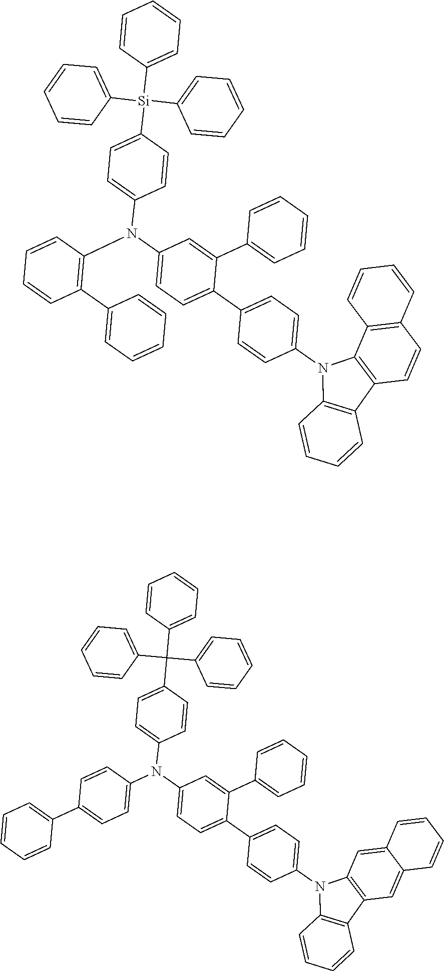 [SMILES: c1ccc(-c2cc(N(c3ccc([Si](c4ccccc4)(c4ccccc4)c4ccccc4)cc3)c3ccccc3-c3ccccc3)ccc2-c2ccc(-n3c4ccccc4c4ccc5ccccc5c43)cc2)cc1 ; c1ccc(-c2ccc(N(c3ccc(C(c4ccccc4)(c4ccccc4)c4ccccc4)cc3)c3ccc(-c4ccc(-n5c6ccccc6c6cc7ccccc7cc65)cc4)c(-c4ccccc4)c3)cc2)cc1] 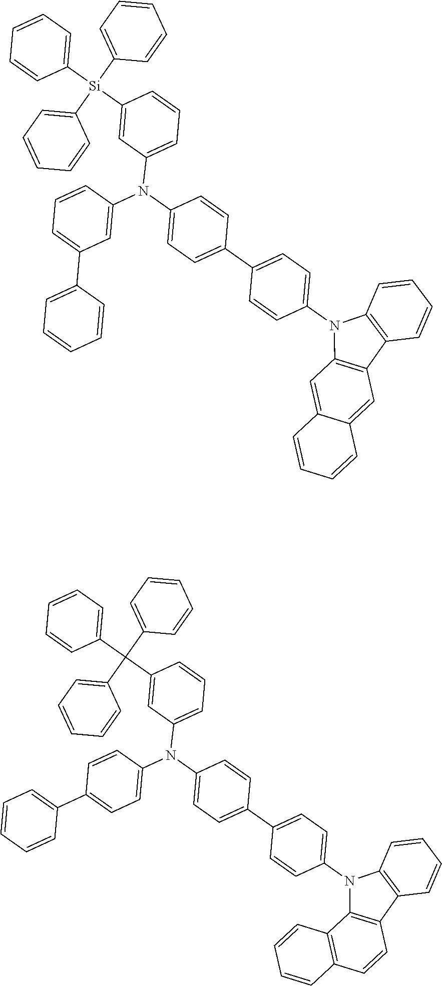 [SMILES: c1ccc(-c2cccc(N(c3ccc(-c4ccc(-n5c6ccccc6c6cc7ccccc7cc65)cc4)cc3)c3cccc([Si](c4ccccc4)(c4ccccc4)c4ccccc4)c3)c2)cc1 ; c1ccc(-c2ccc(N(c3ccc(-c4ccc(-n5c6ccccc6c6ccc7ccccc7c65)cc4)cc3)c3cccc(C(c4ccccc4)(c4ccccc4)c4ccccc4)c3)cc2)cc1] 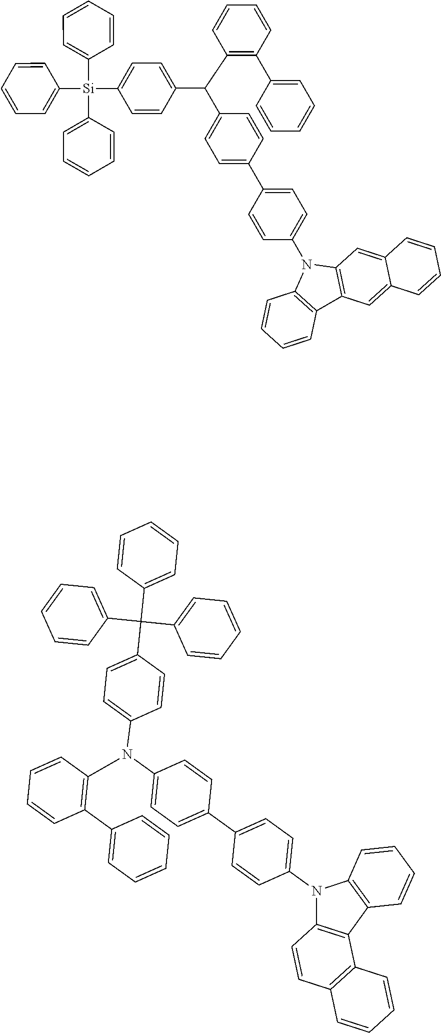 [SMILES: c1ccc(-c2ccccc2C(c2ccc(-c3ccc(-n4c5ccccc5c5cc6ccccc6cc54)cc3)cc2)c2ccc([Si](c3ccccc3)(c3ccccc3)c3ccccc3)cc2)cc1 ; c1ccc(-c2ccccc2N(c2ccc(-c3ccc(-n4c5ccccc5c5c6ccccc6ccc54)cc3)cc2)c2ccc(C(c3ccccc3)(c3ccccc3)c3ccccc3)cc2)cc1] 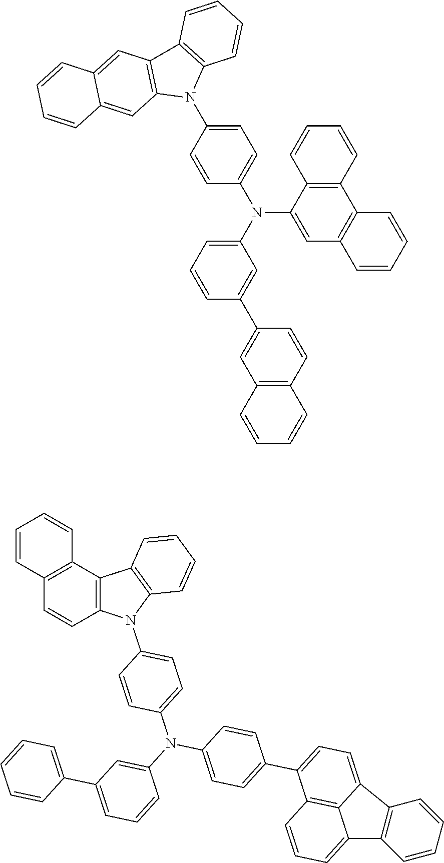 [SMILES: c1cc(-c2ccc3ccccc3c2)cc(N(c2ccc(-n3c4ccccc4c4cc5ccccc5cc43)cc2)c2cc3ccccc3c3ccccc23)c1 ; c1ccc(-c2cccc(N(c3ccc(-c4ccc5c6c(cccc46)-c4ccccc4-5)cc3)c3ccc(-n4c5ccccc5c5c6ccccc6ccc54)cc3)c2)cc1] 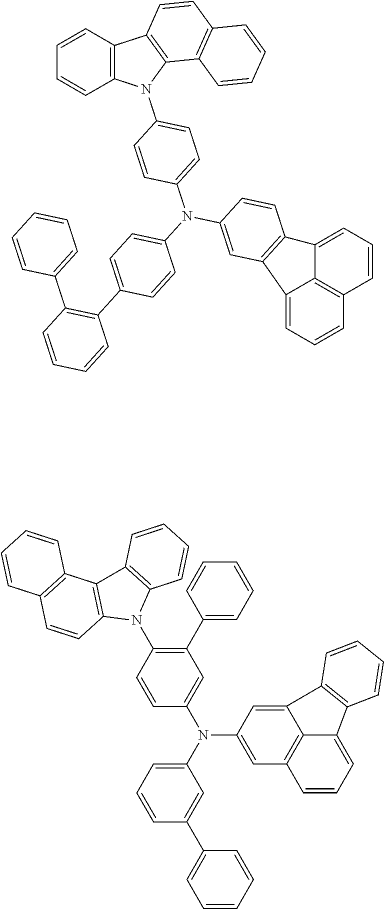 [SMILES: c1ccc(-c2ccccc2-c2ccc(N(c3ccc(-n4c5ccccc5c5ccc6ccccc6c54)cc3)c3ccc4c(c3)-c3cccc5cccc-4c35)cc2)cc1 ; c1ccc(-c2cccc(N(c3ccc(-n4c5ccccc5c5c6ccccc6ccc54)c(-c4ccccc4)c3)c3cc4c5c(cccc5c3)-c3ccccc3-4)c2)cc1] 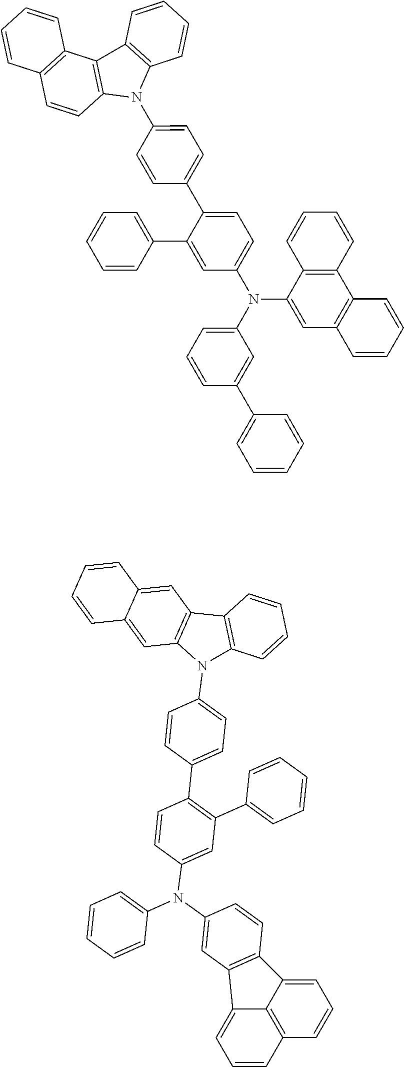 [SMILES: c1ccc(-c2cccc(N(c3ccc(-c4ccc(-n5c6ccccc6c6c7ccccc7ccc65)cc4)c(-c4ccccc4)c3)c3cc4ccccc4c4ccccc34)c2)cc1 ; c1ccc(-c2cc(N(c3ccccc3)c3ccc4c(c3)-c3cccc5cccc-4c35)ccc2-c2ccc(-n3c4ccccc4c4cc5ccccc5cc43)cc2)cc1] 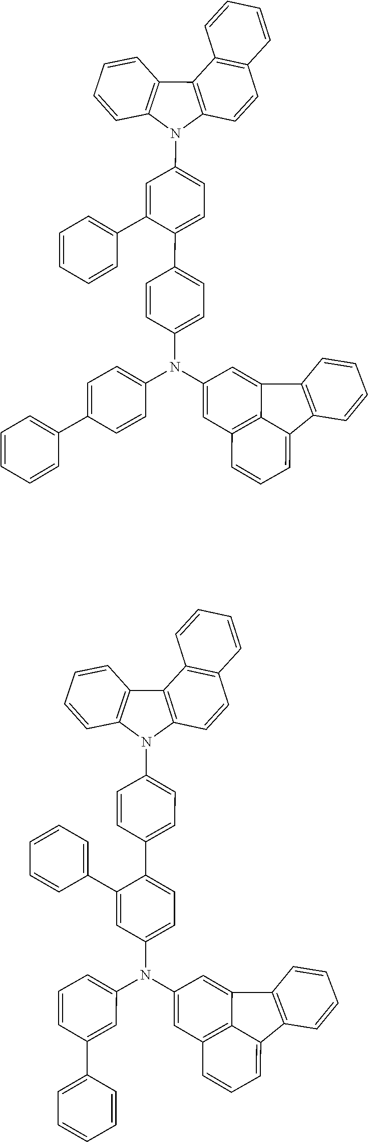 [SMILES: c1ccc(-c2ccc(N(c3ccc(-c4ccc(-n5c6ccccc6c6c7ccccc7ccc65)cc4-c4ccccc4)cc3)c3cc4c5c(cccc5c3)-c3ccccc3-4)cc2)cc1 ; c1ccc(-c2cccc(N(c3ccc(-c4ccc(-n5c6ccccc6c6c7ccccc7ccc65)cc4)c(-c4ccccc4)c3)c3cc4c5c(cccc5c3)-c3ccccc3-4)c2)cc1] 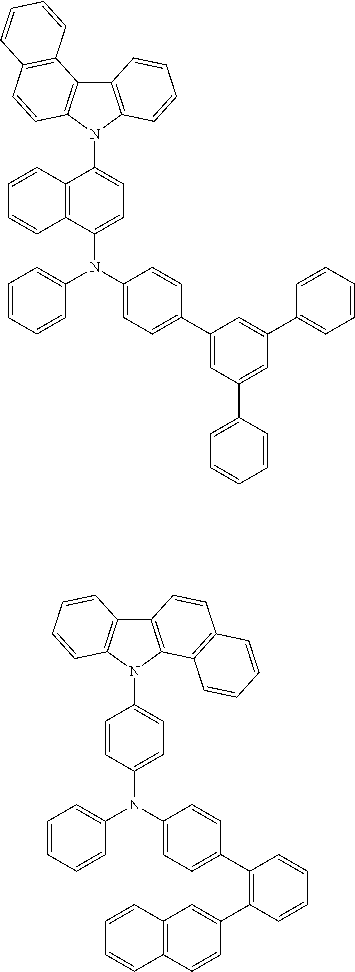 [SMILES: c1ccc(-c2cc(-c3ccccc3)cc(-c3ccc(N(c4ccccc4)c4ccc(-n5c6ccccc6c6c7ccccc7ccc65)c5ccccc45)cc3)c2)cc1 ; c1ccc(N(c2ccc(-c3ccccc3-c3ccc4ccccc4c3)cc2)c2ccc(-n3c4ccccc4c4ccc5ccccc5c43)cc2)cc1] 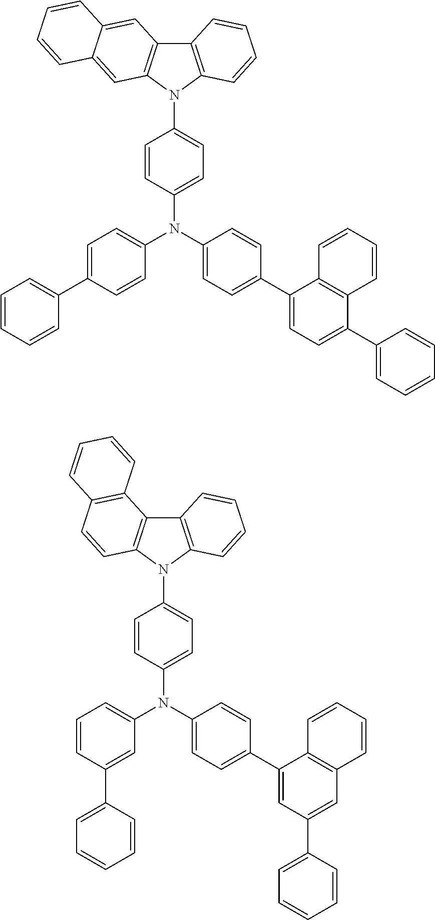 [SMILES: c1ccc(-c2ccc(N(c3ccc(-c4ccc(-c5ccccc5)c5ccccc45)cc3)c3ccc(-n4c5ccccc5c5cc6ccccc6cc54)cc3)cc2)cc1 ; c1ccc(-c2cccc(N(c3ccc(-c4cc(-c5ccccc5)cc5ccccc45)cc3)c3ccc(-n4c5ccccc5c5c6ccccc6ccc54)cc3)c2)cc1] 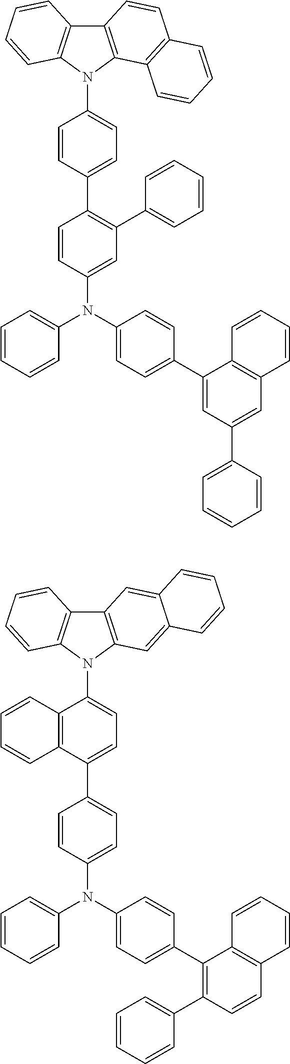 [SMILES: c1ccc(-c2cc(-c3ccc(N(c4ccccc4)c4ccc(-c5ccc(-n6c7ccccc7c7ccc8ccccc8c76)cc5)c(-c5ccccc5)c4)cc3)c3ccccc3c2)cc1 ; c1ccc(-c2ccc3ccccc3c2-c2ccc(N(c3ccccc3)c3ccc(-c4ccc(-n5c6ccccc6c6cc7ccccc7cc65)c5ccccc45)cc3)cc2)cc1] 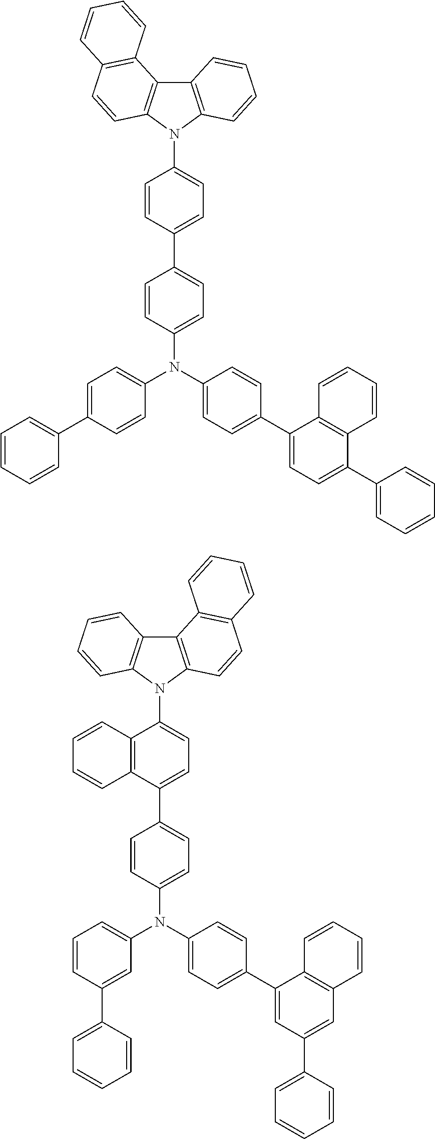 [SMILES: c1ccc(-c2ccc(N(c3ccc(-c4ccc(-n5c6ccccc6c6c7ccccc7ccc65)cc4)cc3)c3ccc(-c4ccc(-c5ccccc5)c5ccccc45)cc3)cc2)cc1 ; c1ccc(-c2cccc(N(c3ccc(-c4cc(-c5ccccc5)cc5ccccc45)cc3)c3ccc(-c4ccc(-n5c6ccccc6c6c7ccccc7ccc65)c5ccccc45)cc3)c2)cc1] 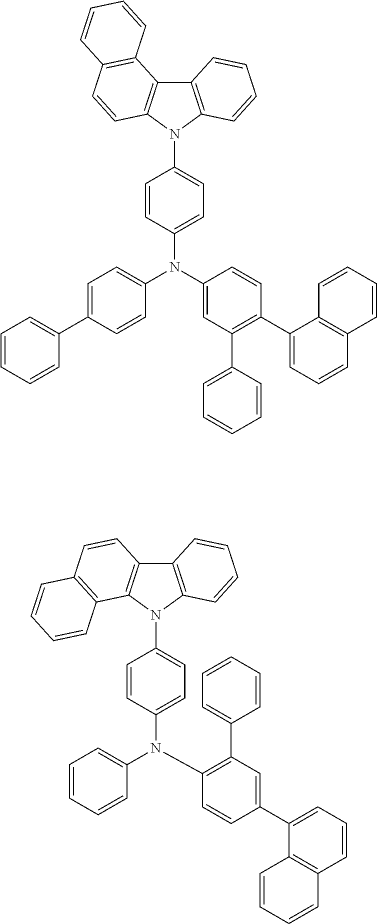 [SMILES: c1ccc(-c2ccc(N(c3ccc(-n4c5ccccc5c5c6ccccc6ccc54)cc3)c3ccc(-c4cccc5ccccc45)c(-c4ccccc4)c3)cc2)cc1 ; c1ccc(-c2cc(-c3cccc4ccccc34)ccc2N(c2ccccc2)c2ccc(-n3c4ccccc4c4ccc5ccccc5c43)cc2)cc1] 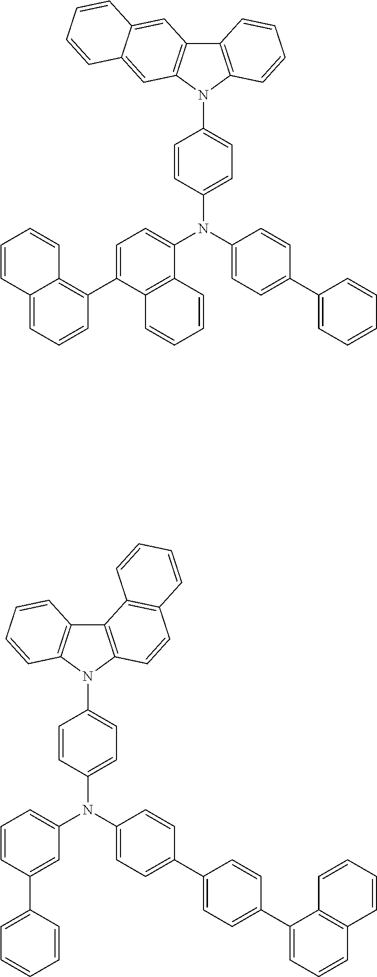 [SMILES: c1ccc(-c2ccc(N(c3ccc(-n4c5ccccc5c5cc6ccccc6cc54)cc3)c3ccc(-c4cccc5ccccc45)c4ccccc34)cc2)cc1 ; c1ccc(-c2cccc(N(c3ccc(-c4ccc(-c5cccc6ccccc56)cc4)cc3)c3ccc(-n4c5ccccc5c5c6ccccc6ccc54)cc3)c2)cc1] 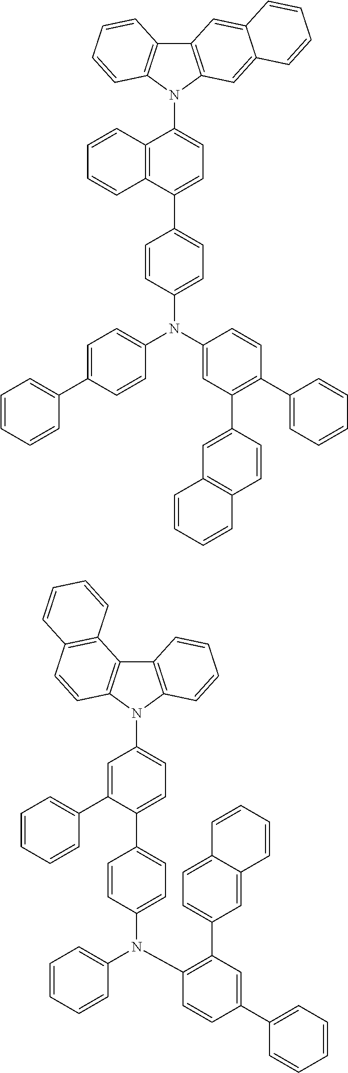 [SMILES: c1ccc(-c2ccc(N(c3ccc(-c4ccc(-n5c6ccccc6c6cc7ccccc7cc65)c5ccccc45)cc3)c3ccc(-c4ccccc4)c(-c4ccc5ccccc5c4)c3)cc2)cc1 ; c1ccc(-c2ccc(N(c3ccccc3)c3ccc(-c4ccc(-n5c6ccccc6c6c7ccccc7ccc65)cc4-c4ccccc4)cc3)c(-c3ccc4ccccc4c3)c2)cc1] 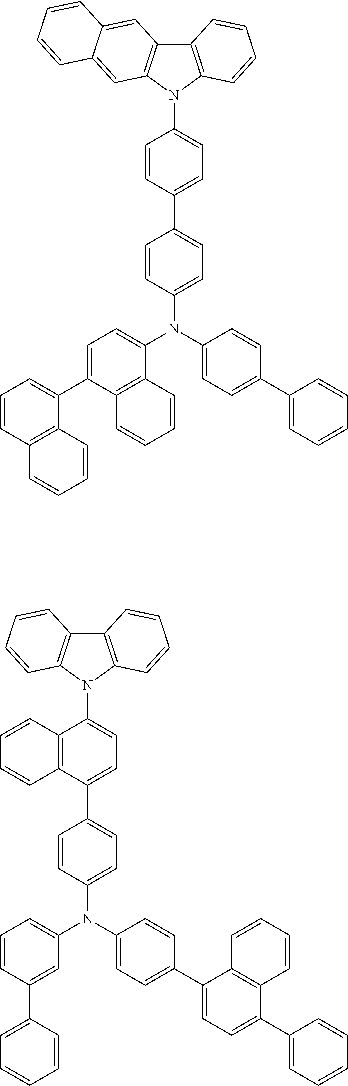 [SMILES: c1ccc(-c2ccc(N(c3ccc(-c4ccc(-n5c6ccccc6c6cc7ccccc7cc65)cc4)cc3)c3ccc(-c4cccc5ccccc45)c4ccccc34)cc2)cc1 ; c1ccc(-c2cccc(N(c3ccc(-c4ccc(-c5ccccc5)c5ccccc45)cc3)c3ccc(-c4ccc(-n5c6ccccc6c6ccccc65)c5ccccc45)cc3)c2)cc1] 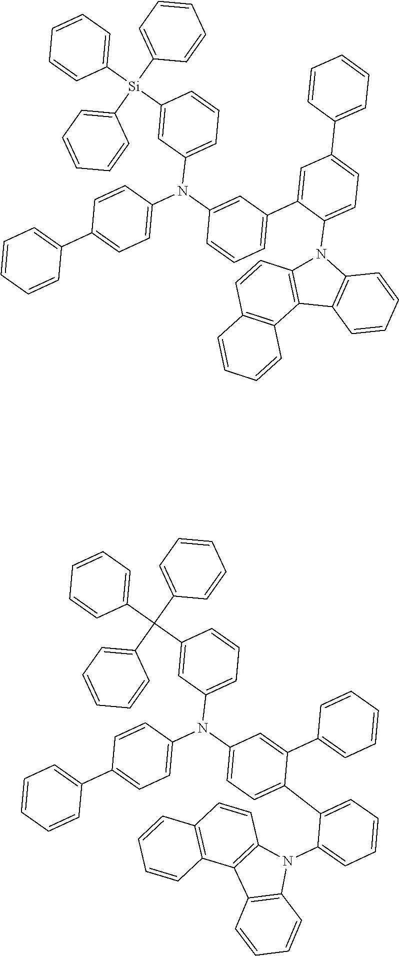 [SMILES: c1ccc(-c2ccc(N(c3cccc(-c4cc(-c5ccccc5)ccc4-n4c5ccccc5c5c6ccccc6ccc54)c3)c3cccc([Si](c4ccccc4)(c4ccccc4)c4ccccc4)c3)cc2)cc1 ; c1ccc(-c2ccc(N(c3cccc(C(c4ccccc4)(c4ccccc4)c4ccccc4)c3)c3ccc(-c4ccccc4-n4c5ccccc5c5c6ccccc6ccc54)c(-c4ccccc4)c3)cc2)cc1] 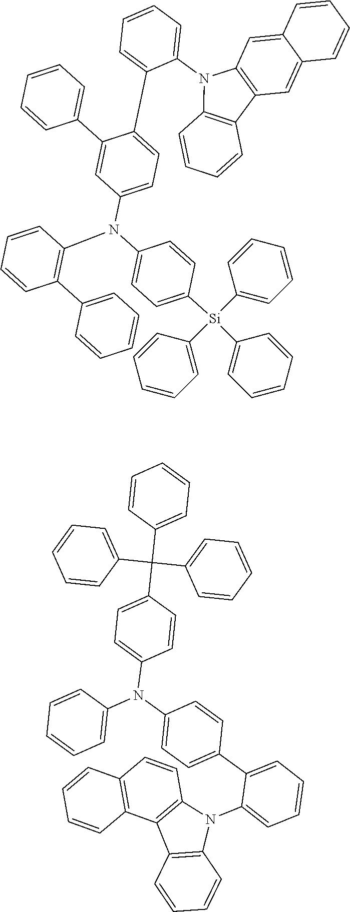 [SMILES: c1ccc(-c2cc(N(c3ccc([Si](c4ccccc4)(c4ccccc4)c4ccccc4)cc3)c3ccccc3-c3ccccc3)ccc2-c2ccccc2-n2c3ccccc3c3cc4ccccc4cc32)cc1 ; c1ccc(N(c2ccc(-c3ccccc3-n3c4ccccc4c4c5ccccc5ccc43)cc2)c2ccc(C(c3ccccc3)(c3ccccc3)c3ccccc3)cc2)cc1] 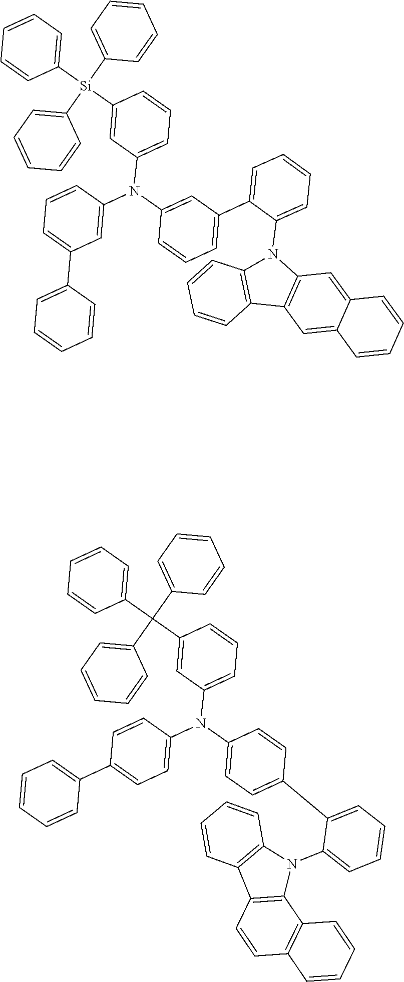 [SMILES: c1ccc(-c2cccc(N(c3cccc(-c4ccccc4-n4c5ccccc5c5cc6ccccc6cc54)c3)c3cccc([Si](c4ccccc4)(c4ccccc4)c4ccccc4)c3)c2)cc1 ; c1ccc(-c2ccc(N(c3ccc(-c4ccccc4-n4c5ccccc5c5ccc6ccccc6c54)cc3)c3cccc(C(c4ccccc4)(c4ccccc4)c4ccccc4)c3)cc2)cc1] 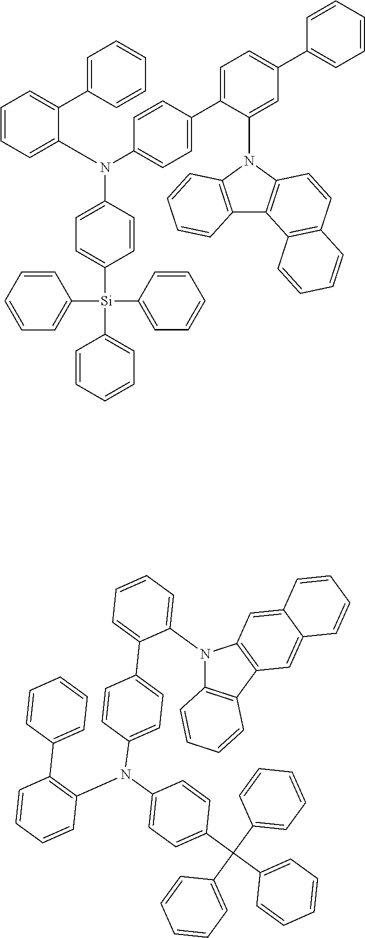 [SMILES: c1ccc(-c2ccc(-c3ccc(N(c4ccc([Si](c5ccccc5)(c5ccccc5)c5ccccc5)cc4)c4ccccc4-c4ccccc4)cc3)c(-n3c4ccccc4c4c5ccccc5ccc43)c2)cc1 ; c1ccc(-c2ccccc2N(c2ccc(-c3ccccc3-n3c4ccccc4c4cc5ccccc5cc43)cc2)c2ccc(C(c3ccccc3)(c3ccccc3)c3ccccc3)cc2)cc1] 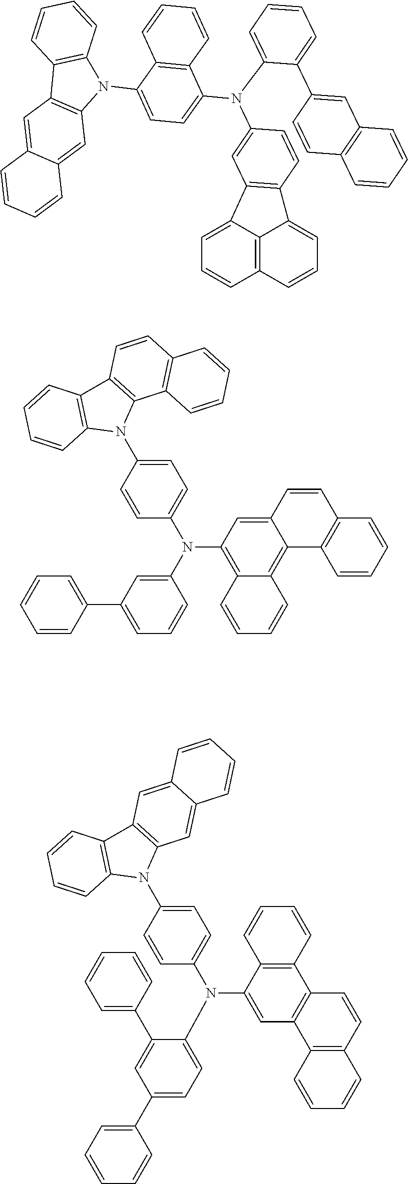 [SMILES: c1ccc(N(c2ccc3c(c2)-c2cccc4cccc-3c24)c2ccc(-n3c4ccccc4c4cc5ccccc5cc43)c3ccccc23)c(-c2ccc3ccccc3c2)c1 ; c1ccc(-c2cccc(N(c3ccc(-n4c5ccccc5c5ccc6ccccc6c54)cc3)c3cc4ccc5ccccc5c4c4ccccc34)c2)cc1 ; c1ccc(-c2ccc(N(c3ccc(-n4c5ccccc5c5cc6ccccc6cc54)cc3)c3cc4c5ccccc5ccc4c4ccccc34)c(-c3ccccc3)c2)cc1] 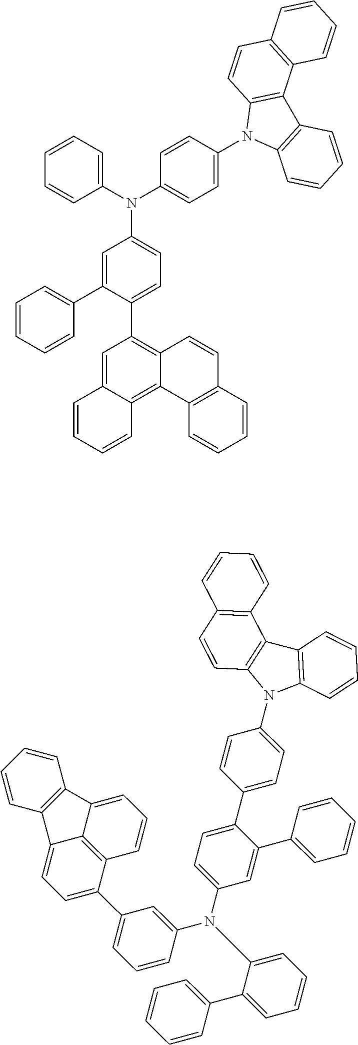 [SMILES: c1ccc(-c2cc(N(c3ccccc3)c3ccc(-n4c5ccccc5c5c6ccccc6ccc54)cc3)ccc2-c2cc3ccccc3c3c2ccc2ccccc23)cc1 ; c1ccc(-c2cc(N(c3cccc(-c4ccc5c6c(cccc46)-c4ccccc4-5)c3)c3ccccc3-c3ccccc3)ccc2-c2ccc(-n3c4ccccc4c4c5ccccc5ccc43)cc2)cc1] 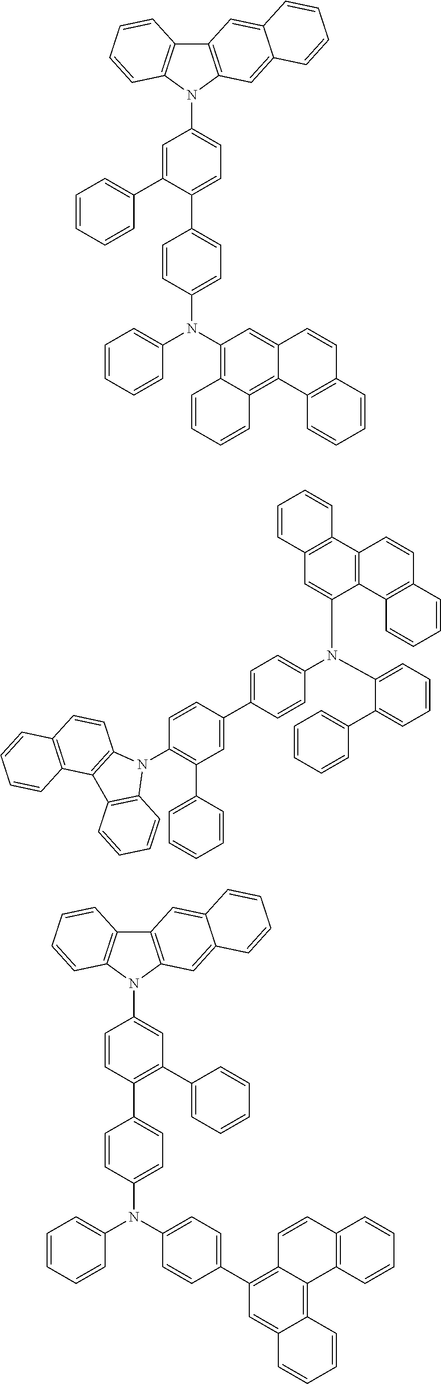 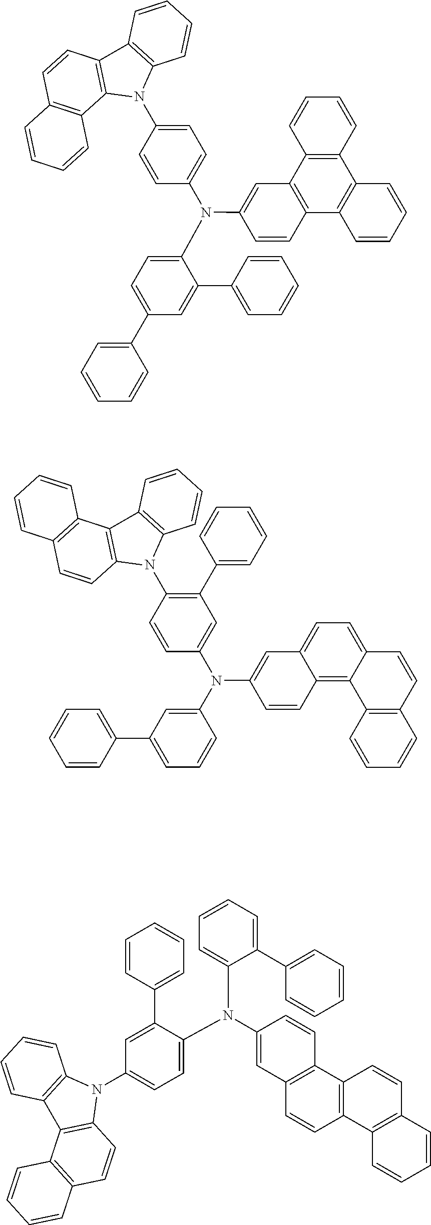 [SMILES: c1ccc(-c2ccc(N(c3ccc(-n4c5ccccc5c5ccc6ccccc6c54)cc3)c3ccc4c5ccccc5c5ccccc5c4c3)c(-c3ccccc3)c2)cc1 ; c1ccc(-c2cccc(N(c3ccc(-n4c5ccccc5c5c6ccccc6ccc54)c(-c4ccccc4)c3)c3ccc4c(ccc5ccc6ccccc6c54)c3)c2)cc1 ; c1ccc(-c2ccccc2N(c2ccc3c(ccc4c5ccccc5ccc34)c2)c2ccc(-n3c4ccccc4c4c5ccccc5ccc43)cc2-c2ccccc2)cc1] 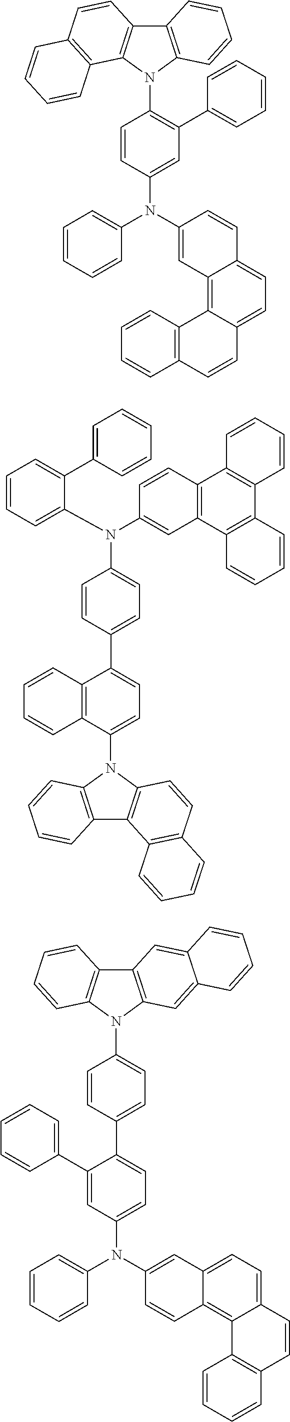 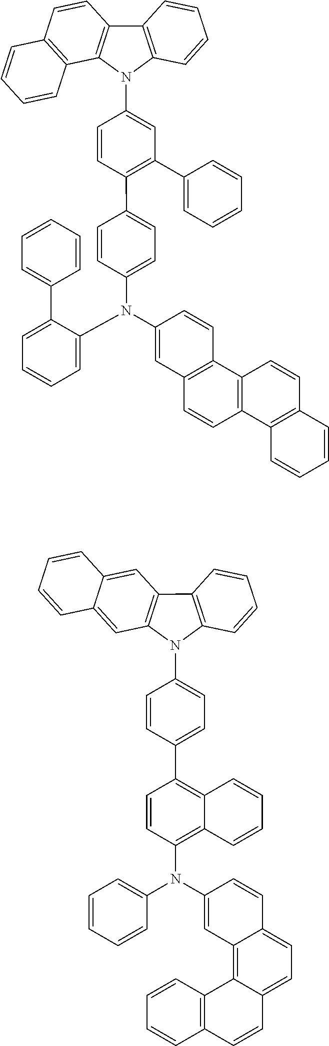 [SMILES: c1ccc(-c2cc(-n3c4ccccc4c4ccc5ccccc5c43)ccc2-c2ccc(N(c3ccc4c(ccc5c6ccccc6ccc45)c3)c3ccccc3-c3ccccc3)cc2)cc1 ; c1ccc(N(c2ccc3ccc4ccc5ccccc5c4c3c2)c2ccc(-c3ccc(-n4c5ccccc5c5cc6ccccc6cc54)cc3)c3ccccc23)cc1] 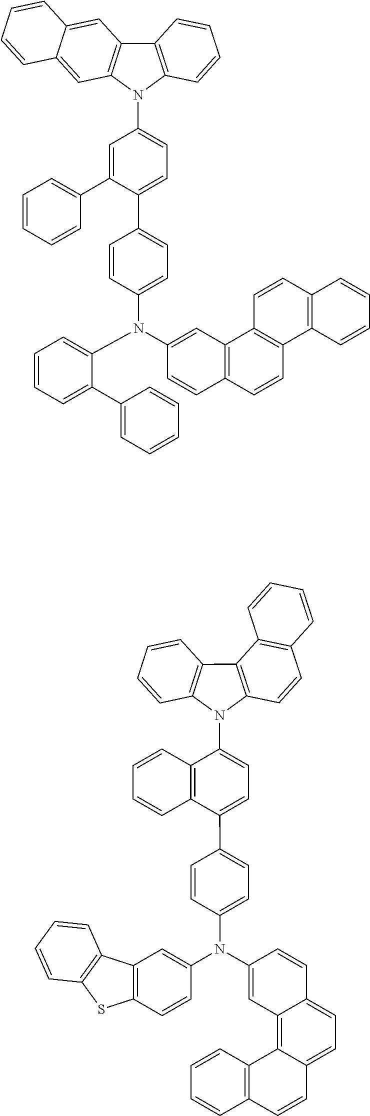 [SMILES: c1ccc(-c2cc(-n3c4ccccc4c4cc5ccccc5cc43)ccc2-c2ccc(N(c3ccc4ccc5c6ccccc6ccc5c4c3)c3ccccc3-c3ccccc3)cc2)cc1 ; c1ccc2c(c1)ccc1ccc3ccc(N(c4ccc(-c5ccc(-n6c7ccccc7c7c8ccccc8ccc76)c6ccccc56)cc4)c4ccc5sc6ccccc6c5c4)cc3c12] 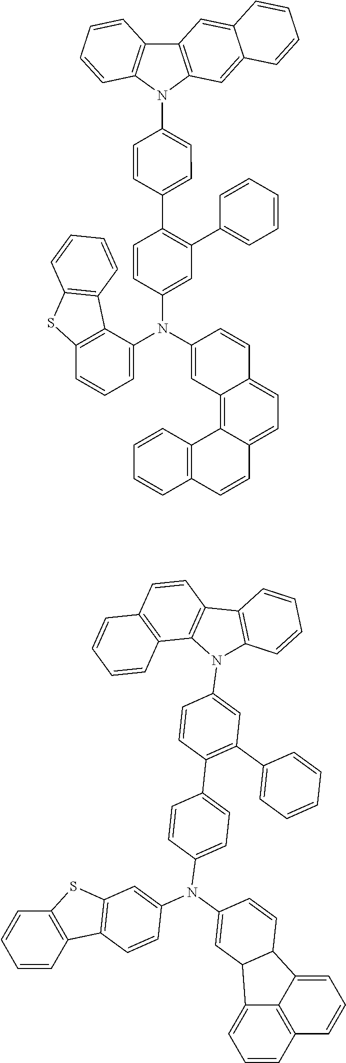 [SMILES: c1ccc(-c2cc(N(c3ccc4ccc5ccc6ccccc6c5c4c3)c3cccc4sc5ccccc5c34)ccc2-c2ccc(-n3c4ccccc4c4cc5ccccc5cc43)cc2)cc1 ; C1=CC2c3cccc4cccc(c34)C2C=C1N(c1ccc(-c2ccc(-n3c4ccccc4c4ccc5ccccc5c43)cc2-c2ccccc2)cc1)c1ccc2c(c1)sc1ccccc12] 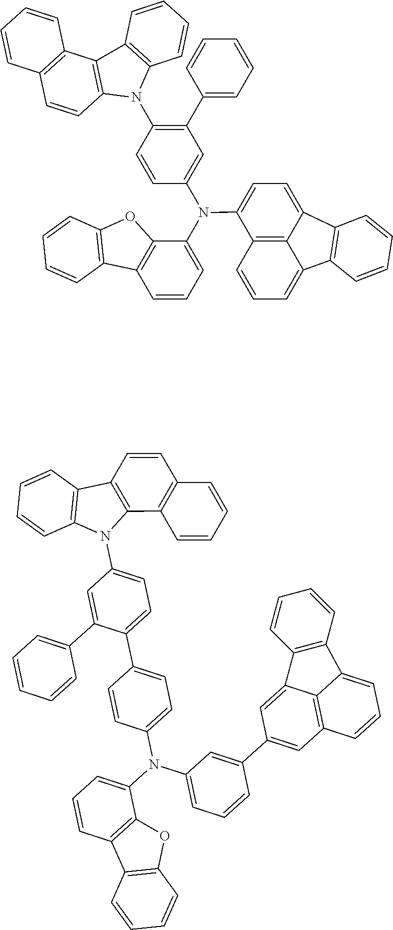 [SMILES: c1ccc(-c2cc(N(c3ccc4c5c(cccc35)-c3ccccc3-4)c3cccc4c3oc3ccccc34)ccc2-n2c3ccccc3c3c4ccccc4ccc32)cc1 ; c1ccc(-c2cc(-n3c4ccccc4c4ccc5ccccc5c43)ccc2-c2ccc(N(c3cccc(-c4cc5c6c(cccc6c4)-c4ccccc4-5)c3)c3cccc4c3oc3ccccc34)cc2)cc1] 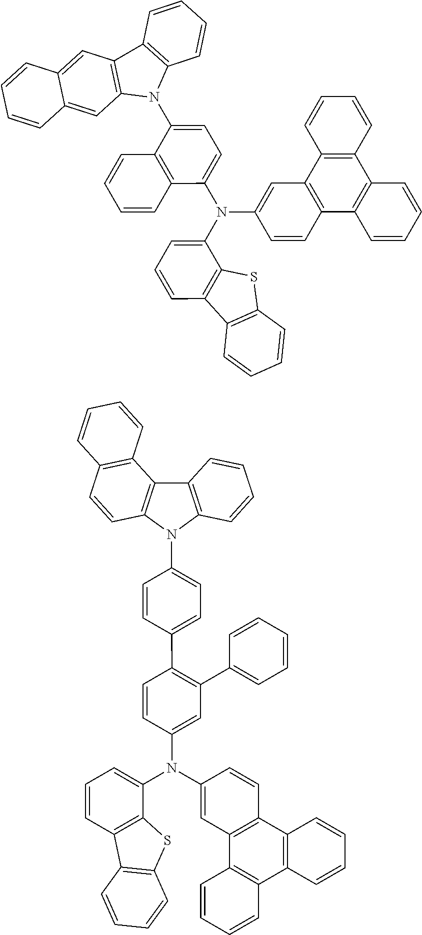 [SMILES: c1ccc2cc3c(cc2c1)c1ccccc1n3-c1ccc(N(c2ccc3c4ccccc4c4ccccc4c3c2)c2cccc3c2sc2ccccc23)c2ccccc12 ; c1ccc(-c2cc(N(c3ccc4c5ccccc5c5ccccc5c4c3)c3cccc4c3sc3ccccc34)ccc2-c2ccc(-n3c4ccccc4c4c5ccccc5ccc43)cc2)cc1] 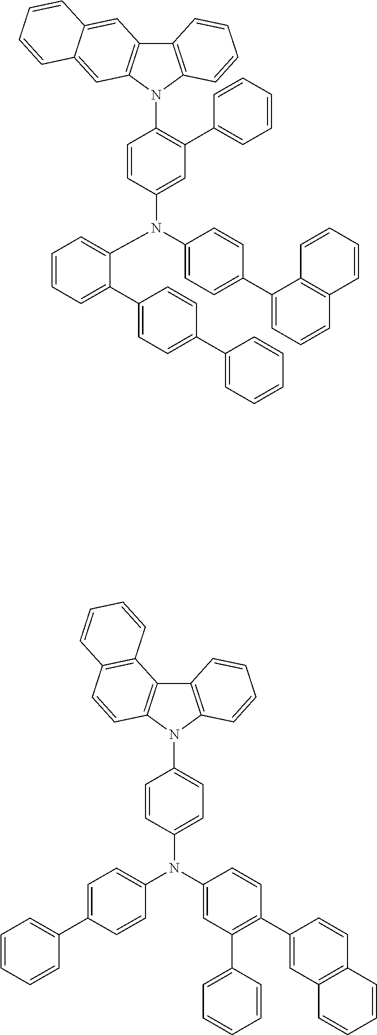 [SMILES: c1ccc(-c2ccc(-c3ccccc3N(c3ccc(-c4cccc5ccccc45)cc3)c3ccc(-n4c5ccccc5c5cc6ccccc6cc54)c(-c4ccccc4)c3)cc2)cc1 ; c1ccc(-c2ccc(N(c3ccc(-n4c5ccccc5c5c6ccccc6ccc54)cc3)c3ccc(-c4ccc5ccccc5c4)c(-c4ccccc4)c3)cc2)cc1] 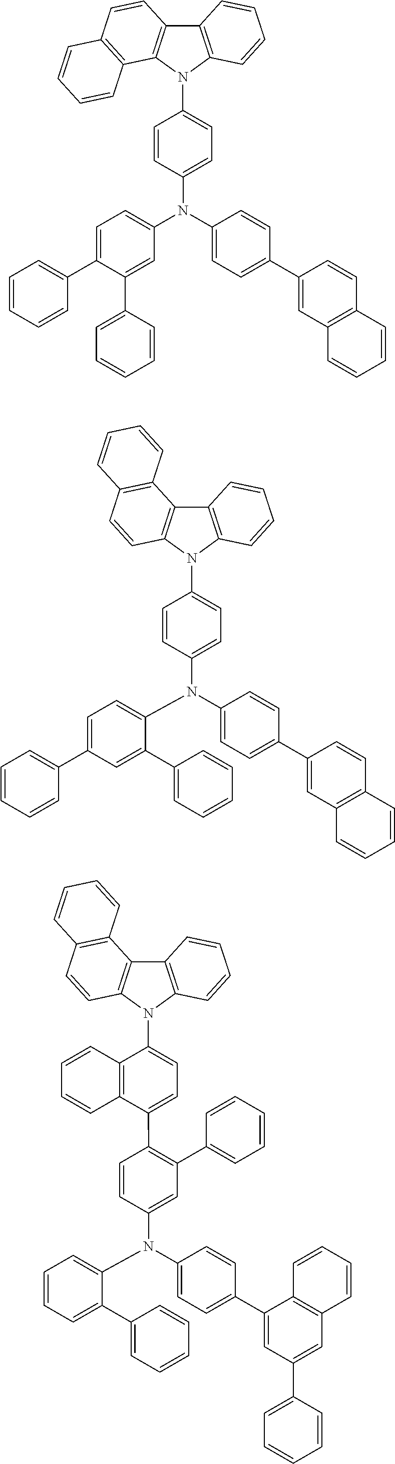 [SMILES: c1ccc(-c2ccc(N(c3ccc(-c4ccc5ccccc5c4)cc3)c3ccc(-n4c5ccccc5c5ccc6ccccc6c54)cc3)cc2-c2ccccc2)cc1 ; c1ccc(-c2ccc(N(c3ccc(-c4ccc5ccccc5c4)cc3)c3ccc(-n4c5ccccc5c5c6ccccc6ccc54)cc3)c(-c3ccccc3)c2)cc1 ; c1ccc(-c2cc(-c3ccc(N(c4ccc(-c5ccc(-n6c7ccccc7c7c8ccccc8ccc76)c6ccccc56)c(-c5ccccc5)c4)c4ccccc4-c4ccccc4)cc3)c3ccccc3c2)cc1] 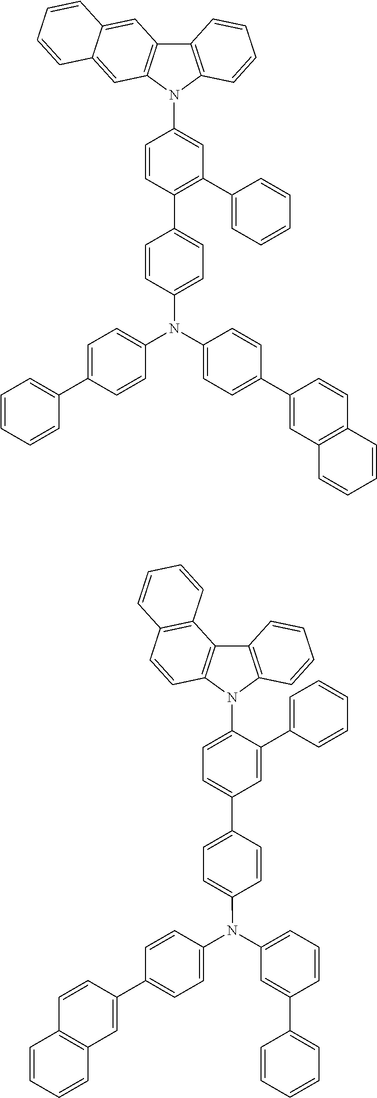 [SMILES: c1ccc(-c2ccc(N(c3ccc(-c4ccc5ccccc5c4)cc3)c3ccc(-c4ccc(-n5c6ccccc6c6cc7ccccc7cc65)cc4-c4ccccc4)cc3)cc2)cc1 ; c1ccc(-c2cccc(N(c3ccc(-c4ccc(-n5c6ccccc6c6c7ccccc7ccc65)c(-c5ccccc5)c4)cc3)c3ccc(-c4ccc5ccccc5c4)cc3)c2)cc1] 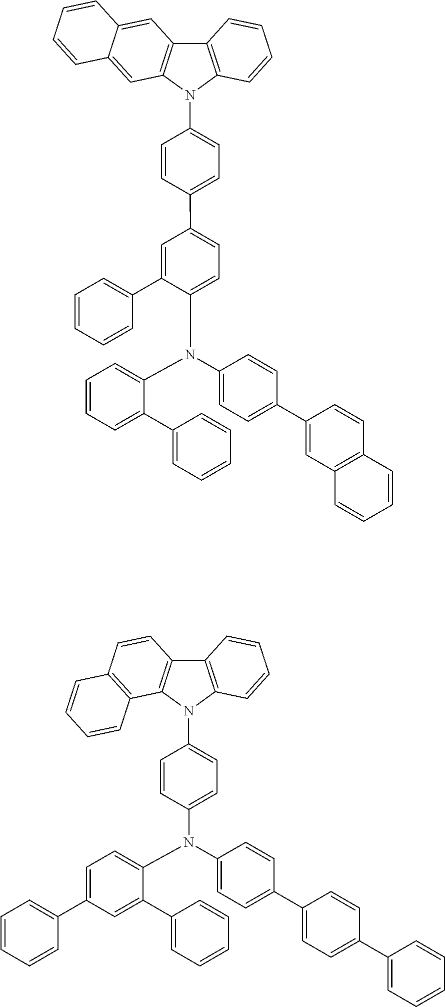 [SMILES: c1ccc(-c2ccccc2N(c2ccc(-c3ccc4ccccc4c3)cc2)c2ccc(-c3ccc(-n4c5ccccc5c5cc6ccccc6cc54)cc3)cc2-c2ccccc2)cc1 ; c1ccc(-c2ccc(-c3ccc(N(c4ccc(-n5c6ccccc6c6ccc7ccccc7c65)cc4)c4ccc(-c5ccccc5)cc4-c4ccccc4)cc3)cc2)cc1] 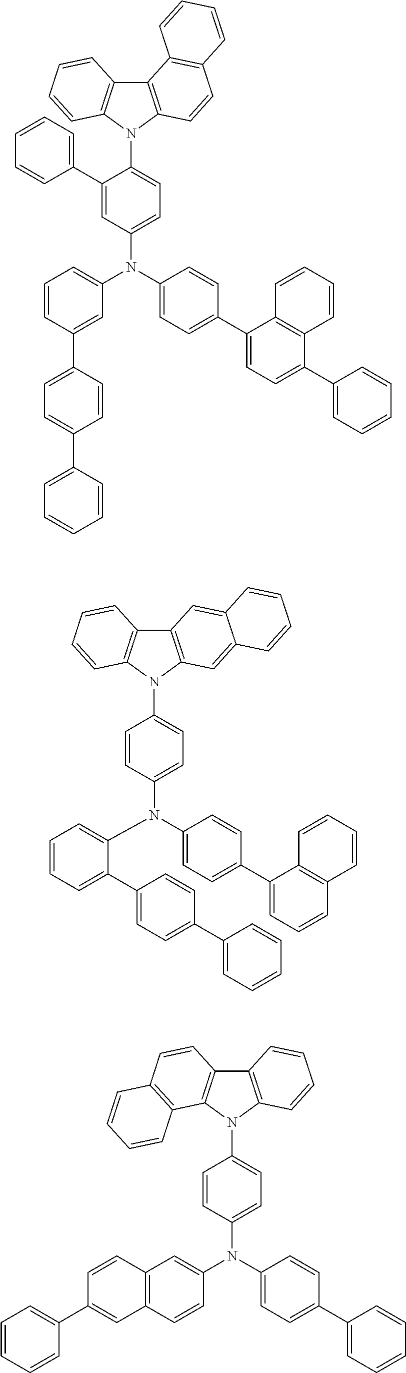 [SMILES: c1ccc(-c2ccc(-c3cccc(N(c4ccc(-c5ccc(-c6ccccc6)c6ccccc56)cc4)c4ccc(-n5c6ccccc6c6c7ccccc7ccc65)c(-c5ccccc5)c4)c3)cc2)cc1 ; c1ccc(-c2ccc(-c3ccccc3N(c3ccc(-c4cccc5ccccc45)cc3)c3ccc(-n4c5ccccc5c5cc6ccccc6cc54)cc3)cc2)cc1 ; c1ccc(-c2ccc(N(c3ccc(-n4c5ccccc5c5ccc6ccccc6c54)cc3)c3ccc4cc(-c5ccccc5)ccc4c3)cc2)cc1] 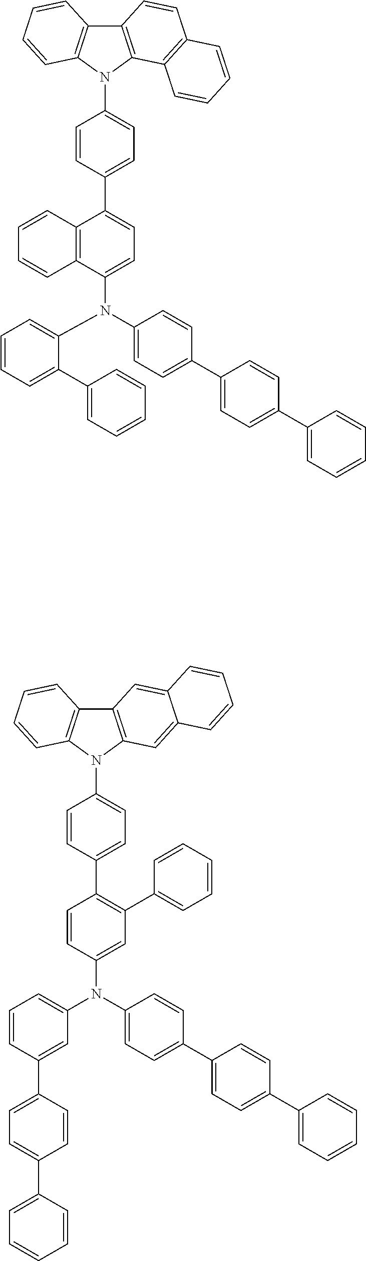 [SMILES: c1ccc(-c2ccc(-c3ccc(N(c4ccccc4-c4ccccc4)c4ccc(-c5ccc(-n6c7ccccc7c7ccc8ccccc8c76)cc5)c5ccccc45)cc3)cc2)cc1 ; c1ccc(-c2ccc(-c3ccc(N(c4cccc(-c5ccc(-c6ccccc6)cc5)c4)c4ccc(-c5ccc(-n6c7ccccc7c7cc8ccccc8cc76)cc5)c(-c5ccccc5)c4)cc3)cc2)cc1] 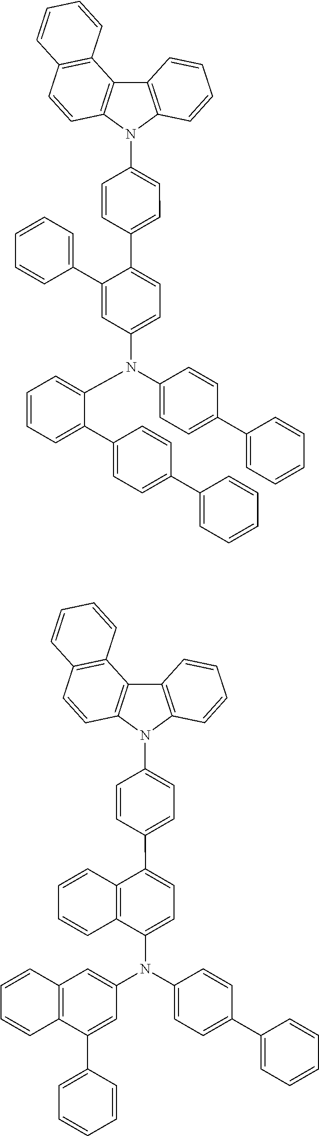 [SMILES: c1ccc(-c2ccc(-c3ccccc3N(c3ccc(-c4ccccc4)cc3)c3ccc(-c4ccc(-n5c6ccccc6c6c7ccccc7ccc65)cc4)c(-c4ccccc4)c3)cc2)cc1 ; c1ccc(-c2ccc(N(c3cc(-c4ccccc4)c4ccccc4c3)c3ccc(-c4ccc(-n5c6ccccc6c6c7ccccc7ccc65)cc4)c4ccccc34)cc2)cc1] 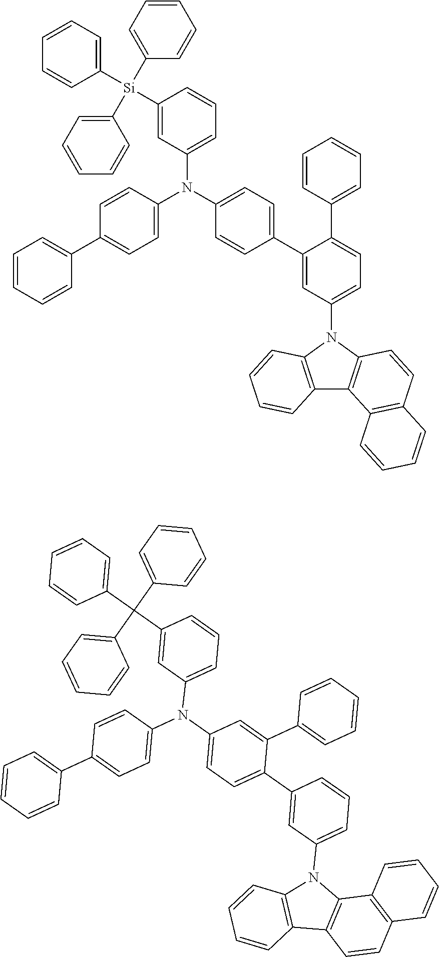 [SMILES: c1ccc(-c2ccc(N(c3ccc(-c4cc(-n5c6ccccc6c6c7ccccc7ccc65)ccc4-c4ccccc4)cc3)c3cccc([Si](c4ccccc4)(c4ccccc4)c4ccccc4)c3)cc2)cc1 ; c1ccc(-c2ccc(N(c3cccc(C(c4ccccc4)(c4ccccc4)c4ccccc4)c3)c3ccc(-c4cccc(-n5c6ccccc6c6ccc7ccccc7c65)c4)c(-c4ccccc4)c3)cc2)cc1] 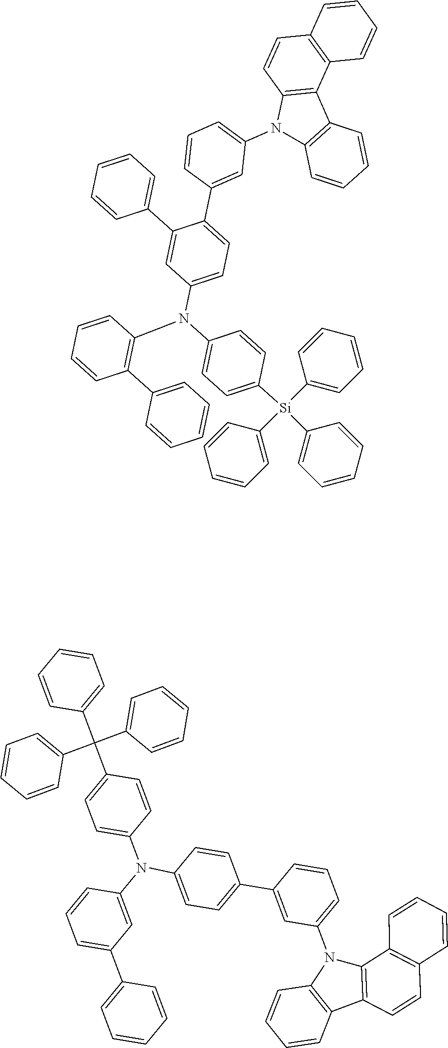 [SMILES: c1ccc(-c2cc(N(c3ccc([Si](c4ccccc4)(c4ccccc4)c4ccccc4)cc3)c3ccccc3-c3ccccc3)ccc2-c2cccc(-n3c4ccccc4c4c5ccccc5ccc43)c2)cc1 ; c1ccc(-c2cccc(N(c3ccc(-c4cccc(-n5c6ccccc6c6ccc7ccccc7c65)c4)cc3)c3ccc(C(c4ccccc4)(c4ccccc4)c4ccccc4)cc3)c2)cc1] 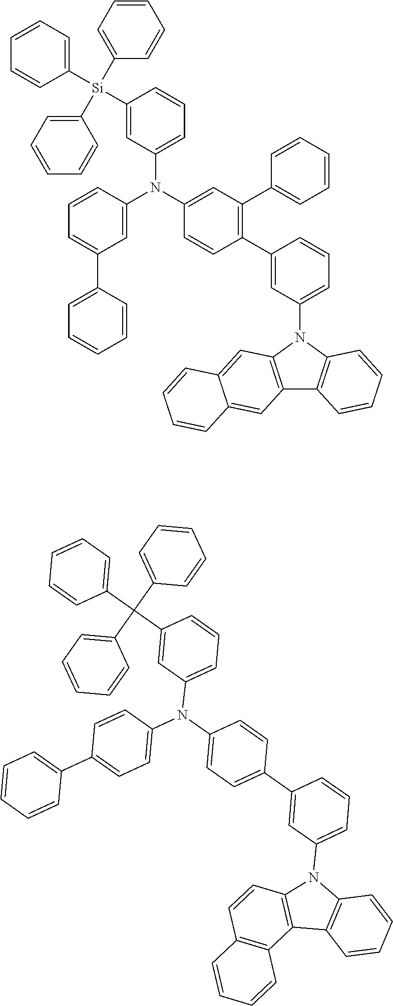 [SMILES: c1ccc(-c2cccc(N(c3cccc([Si](c4ccccc4)(c4ccccc4)c4ccccc4)c3)c3ccc(-c4cccc(-n5c6ccccc6c6cc7ccccc7cc65)c4)c(-c4ccccc4)c3)c2)cc1 ; c1ccc(-c2ccc(N(c3ccc(-c4cccc(-n5c6ccccc6c6c7ccccc7ccc65)c4)cc3)c3cccc(C(c4ccccc4)(c4ccccc4)c4ccccc4)c3)cc2)cc1] 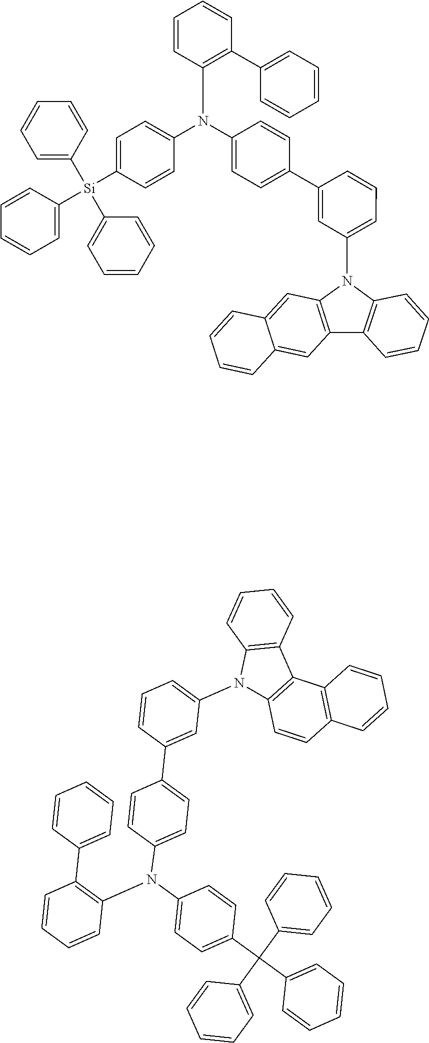 [SMILES: c1ccc(-c2ccccc2N(c2ccc(-c3cccc(-n4c5ccccc5c5cc6ccccc6cc54)c3)cc2)c2ccc([Si](c3ccccc3)(c3ccccc3)c3ccccc3)cc2)cc1 ; c1ccc(-c2ccccc2N(c2ccc(-c3cccc(-n4c5ccccc5c5c6ccccc6ccc54)c3)cc2)c2ccc(C(c3ccccc3)(c3ccccc3)c3ccccc3)cc2)cc1] 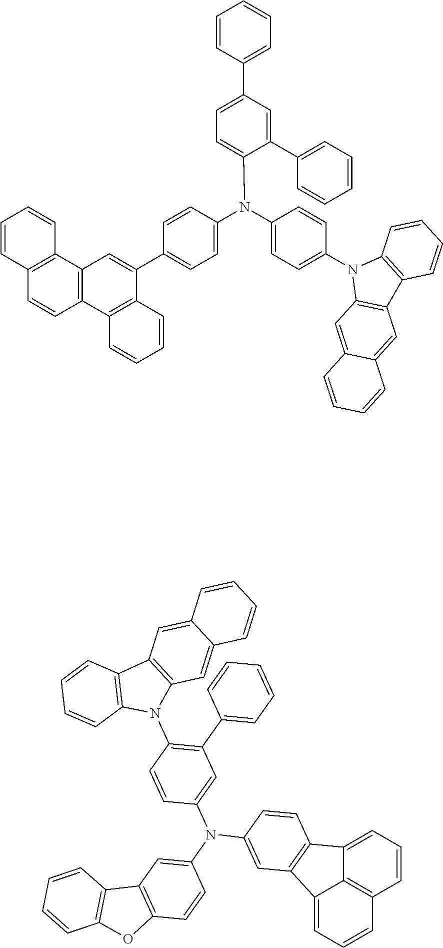 [SMILES: c1ccc(-c2ccc(N(c3ccc(-c4cc5c6ccccc6ccc5c5ccccc45)cc3)c3ccc(-n4c5ccccc5c5cc6ccccc6cc54)cc3)c(-c3ccccc3)c2)cc1 ; c1ccc(-c2cc(N(c3ccc4c(c3)-c3cccc5cccc-4c35)c3ccc4oc5ccccc5c4c3)ccc2-n2c3ccccc3c3cc4ccccc4cc32)cc1] 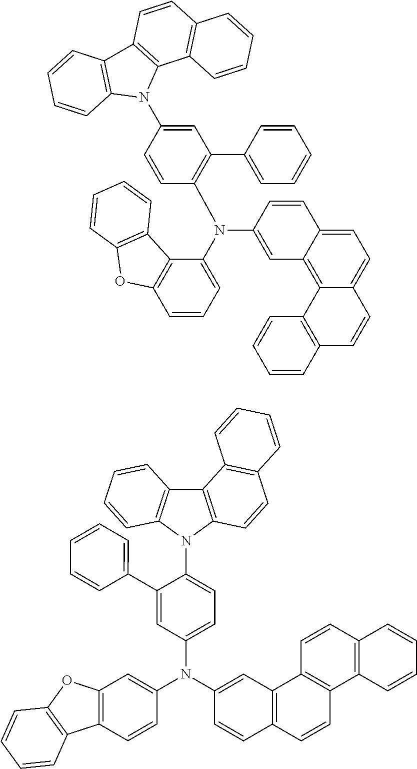 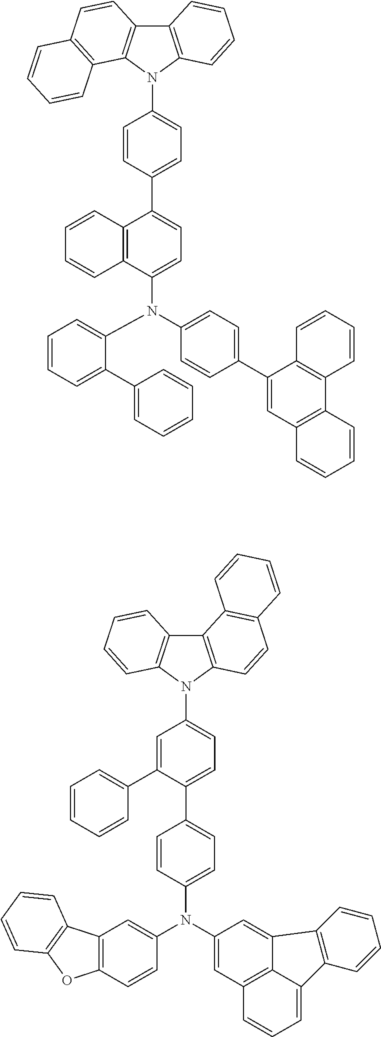 [SMILES: c1ccc(-c2ccccc2N(c2ccc(-c3cc4ccccc4c4ccccc34)cc2)c2ccc(-c3ccc(-n4c5ccccc5c5ccc6ccccc6c54)cc3)c3ccccc23)cc1 ; c1ccc(-c2cc(-n3c4ccccc4c4c5ccccc5ccc43)ccc2-c2ccc(N(c3cc4c5c(cccc5c3)-c3ccccc3-4)c3ccc4oc5ccccc5c4c3)cc2)cc1] 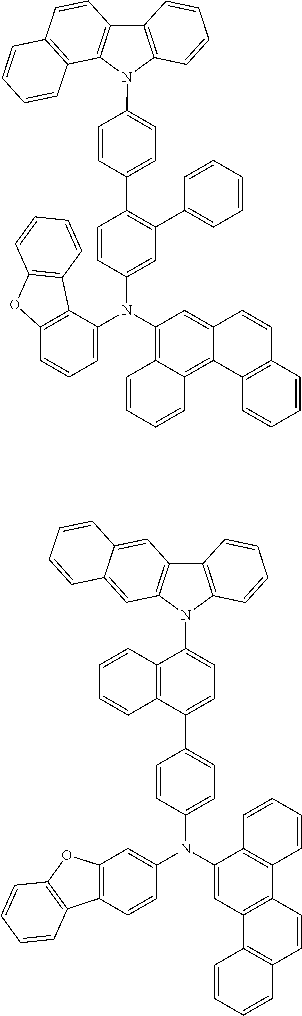 [SMILES: c1ccc(-c2cc(N(c3cc4ccc5ccccc5c4c4ccccc34)c3cccc4oc5ccccc5c34)ccc2-c2ccc(-n3c4ccccc4c4ccc5ccccc5c43)cc2)cc1 ; c1ccc2cc3c(cc2c1)c1ccccc1n3-c1ccc(-c2ccc(N(c3ccc4c(c3)oc3ccccc34)c3cc4c5ccccc5ccc4c4ccccc34)cc2)c2ccccc12] 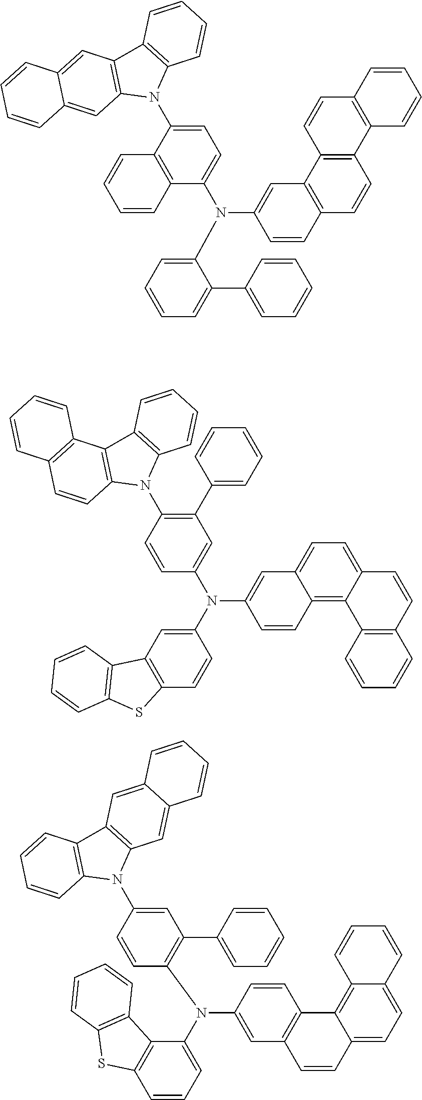 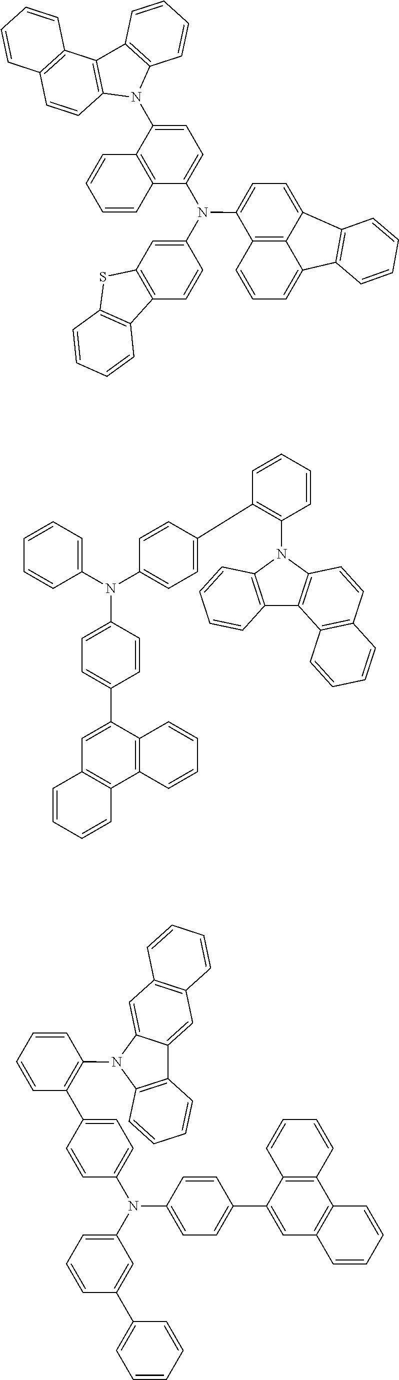 [SMILES: c1ccc2c(c1)-c1cccc3c(N(c4ccc5c(c4)sc4ccccc45)c4ccc(-n5c6ccccc6c6c7ccccc7ccc65)c5ccccc45)ccc-2c13 ; c1ccc(N(c2ccc(-c3ccccc3-n3c4ccccc4c4c5ccccc5ccc43)cc2)c2ccc(-c3cc4ccccc4c4ccccc34)cc2)cc1 ; c1ccc(-c2cccc(N(c3ccc(-c4ccccc4-n4c5ccccc5c5cc6ccccc6cc54)cc3)c3ccc(-c4cc5ccccc5c5ccccc45)cc3)c2)cc1] 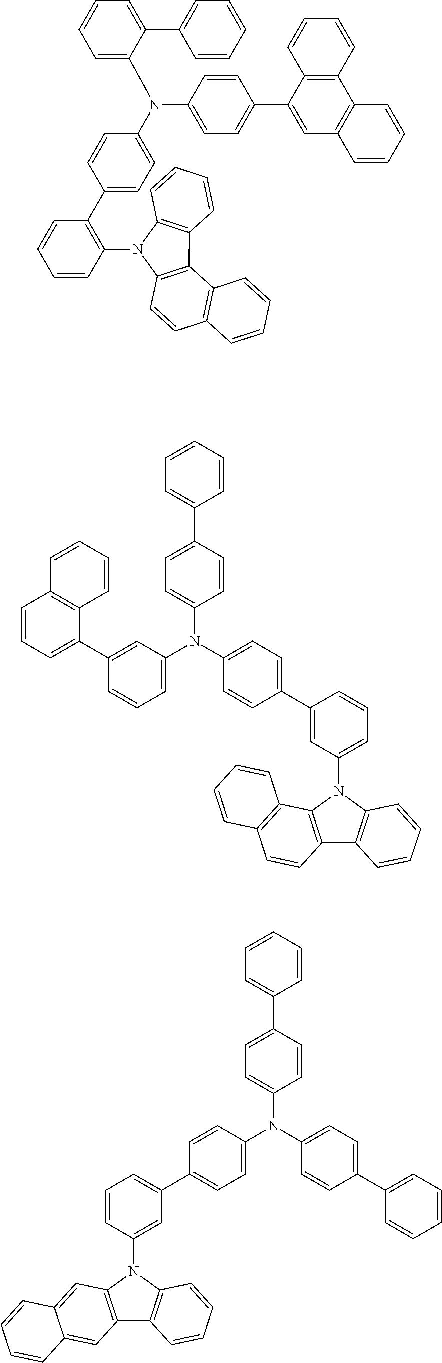 [SMILES: c1ccc(-c2ccccc2N(c2ccc(-c3ccccc3-n3c4ccccc4c4c5ccccc5ccc43)cc2)c2ccc(-c3cc4ccccc4c4ccccc34)cc2)cc1 ; c1ccc(-c2ccc(N(c3ccc(-c4cccc(-n5c6ccccc6c6ccc7ccccc7c65)c4)cc3)c3cccc(-c4cccc5ccccc45)c3)cc2)cc1 ; c1ccc(-c2ccc(N(c3ccc(-c4ccccc4)cc3)c3ccc(-c4cccc(-n5c6ccccc6c6cc7ccccc7cc65)c4)cc3)cc2)cc1] 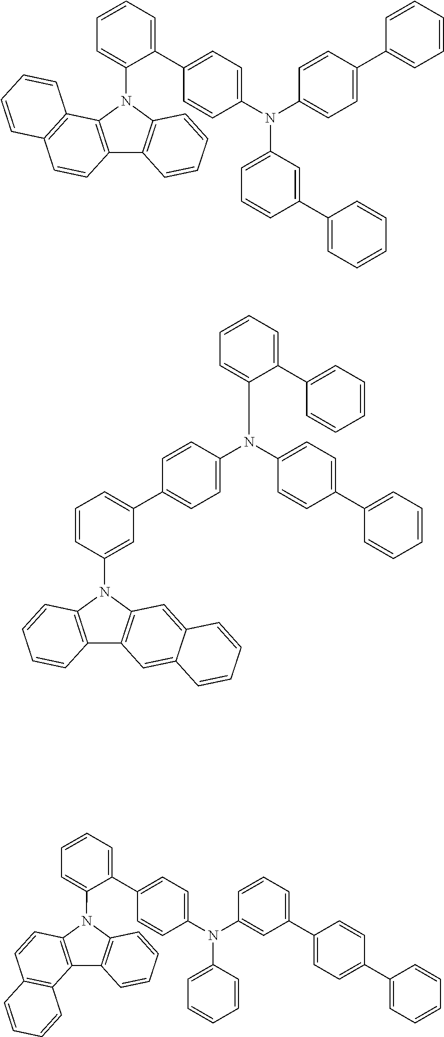 [SMILES: c1ccc(-c2ccc(N(c3ccc(-c4ccccc4-n4c5ccccc5c5ccc6ccccc6c54)cc3)c3cccc(-c4ccccc4)c3)cc2)cc1 ; c1ccc(-c2ccc(N(c3ccc(-c4cccc(-n5c6ccccc6c6cc7ccccc7cc65)c4)cc3)c3ccccc3-c3ccccc3)cc2)cc1 ; c1ccc(-c2ccc(-c3cccc(N(c4ccccc4)c4ccc(-c5ccccc5-n5c6ccccc6c6c7ccccc7ccc65)cc4)c3)cc2)cc1] 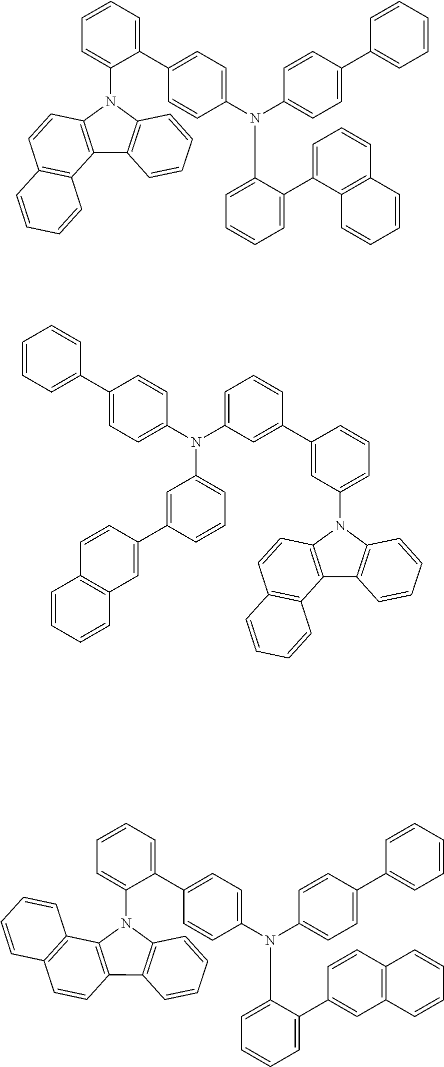 [SMILES: c1ccc(-c2ccc(N(c3ccc(-c4ccccc4-n4c5ccccc5c5c6ccccc6ccc54)cc3)c3ccccc3-c3cccc4ccccc34)cc2)cc1 ; c1ccc(-c2ccc(N(c3cccc(-c4cccc(-n5c6ccccc6c6c7ccccc7ccc65)c4)c3)c3cccc(-c4ccc5ccccc5c4)c3)cc2)cc1 ; c1ccc(-c2ccc(N(c3ccc(-c4ccccc4-n4c5ccccc5c5ccc6ccccc6c54)cc3)c3ccccc3-c3ccc4ccccc4c3)cc2)cc1] 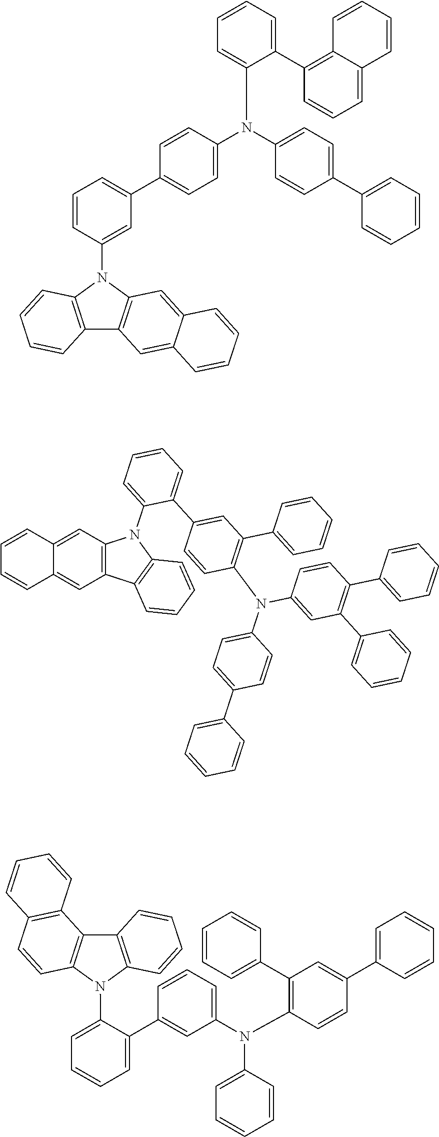 [SMILES: c1ccc(-c2ccc(N(c3ccc(-c4cccc(-n5c6ccccc6c6cc7ccccc7cc65)c4)cc3)c3ccccc3-c3cccc4ccccc34)cc2)cc1 ; c1ccc(-c2ccc(N(c3ccc(-c4ccccc4)c(-c4ccccc4)c3)c3ccc(-c4ccccc4-n4c5ccccc5c5cc6ccccc6cc54)cc3-c3ccccc3)cc2)cc1 ; c1ccc(-c2ccc(N(c3ccccc3)c3cccc(-c4ccccc4-n4c5ccccc5c5c6ccccc6ccc54)c3)c(-c3ccccc3)c2)cc1] 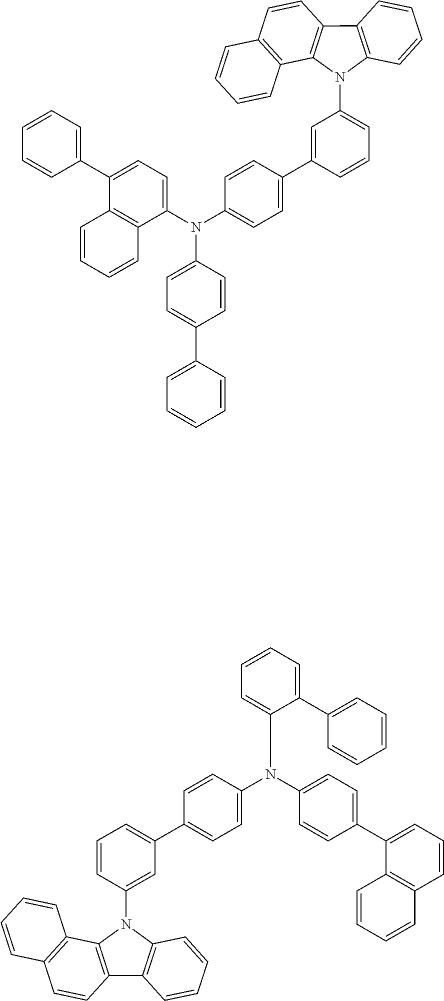 [SMILES: c1ccc(-c2ccc(N(c3ccc(-c4cccc(-n5c6ccccc6c6ccc7ccccc7c65)c4)cc3)c3ccc(-c4ccccc4)c4ccccc34)cc2)cc1 ; c1ccc(-c2ccccc2N(c2ccc(-c3cccc(-n4c5ccccc5c5ccc6ccccc6c54)c3)cc2)c2ccc(-c3cccc4ccccc34)cc2)cc1] 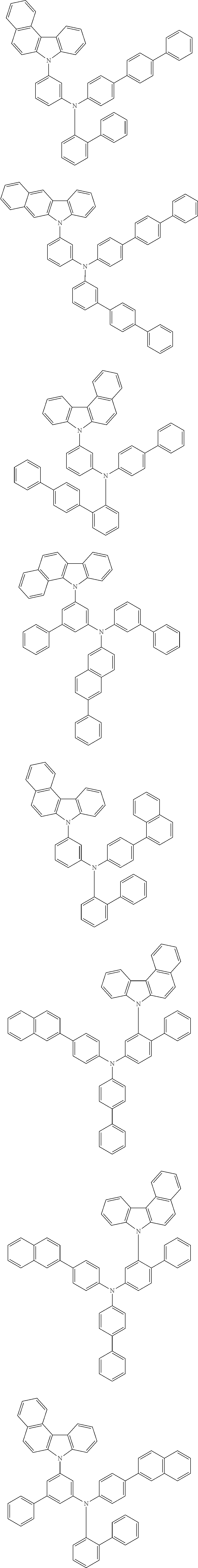 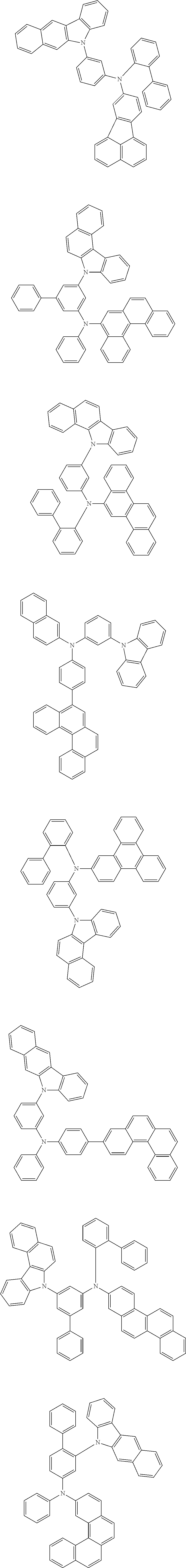 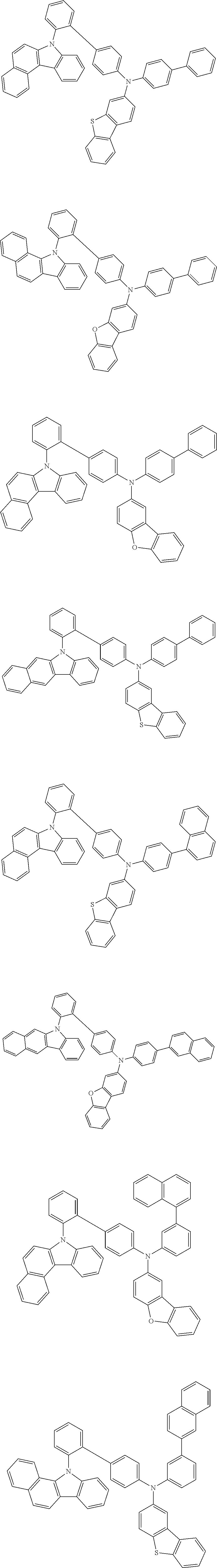 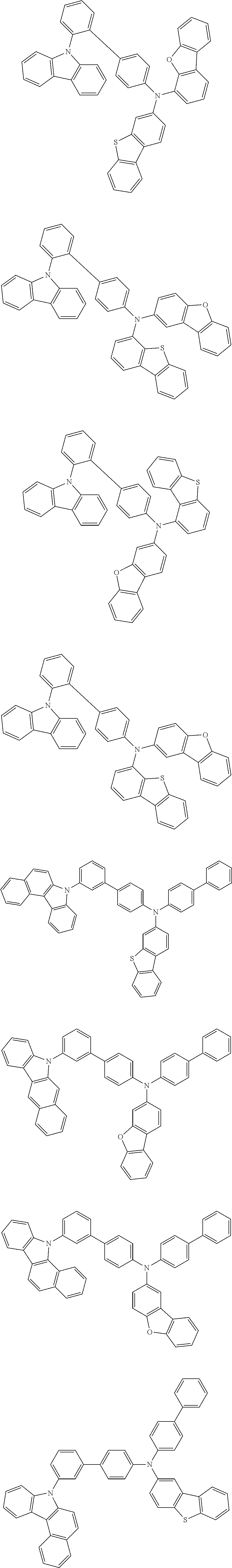 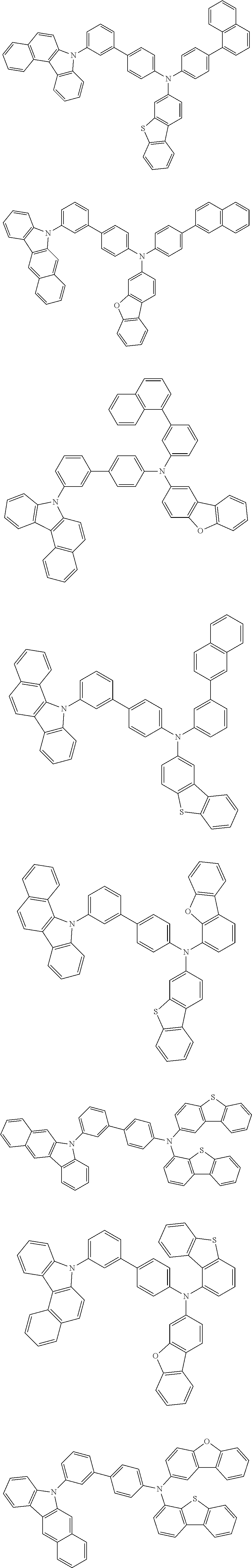 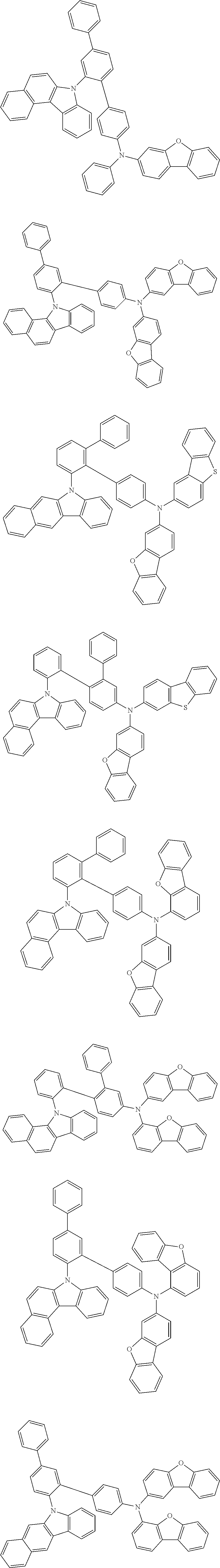 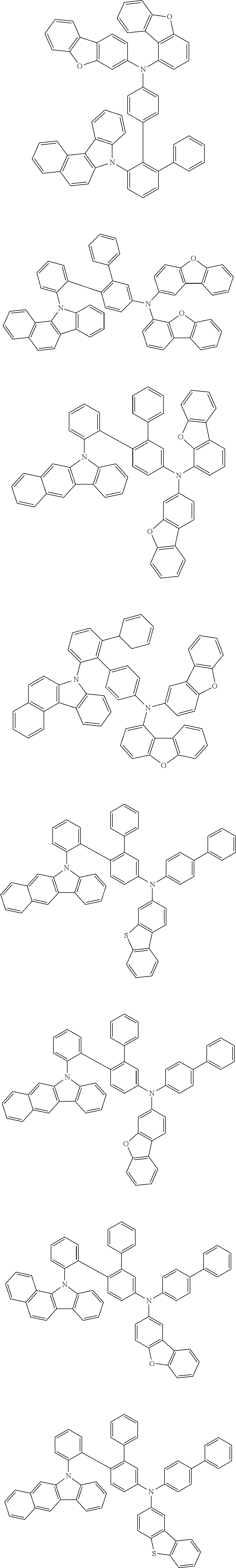 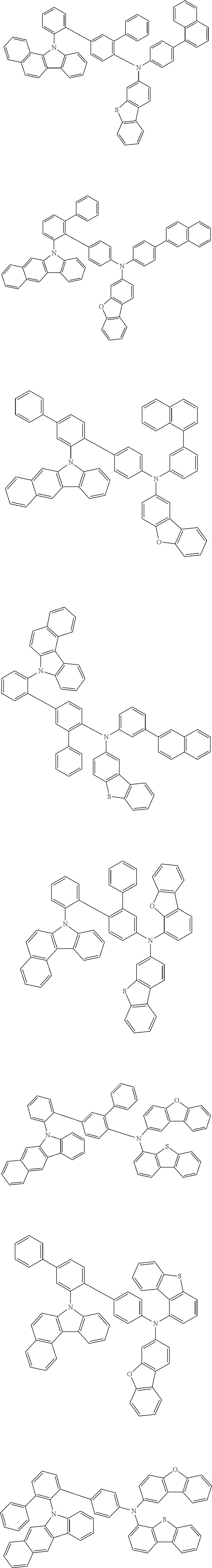 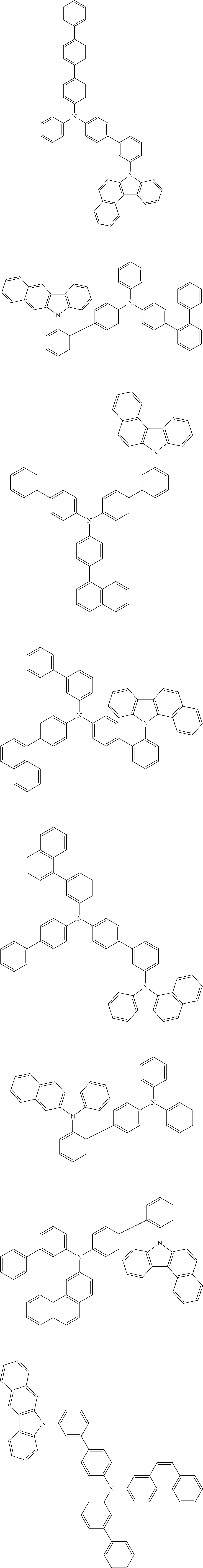 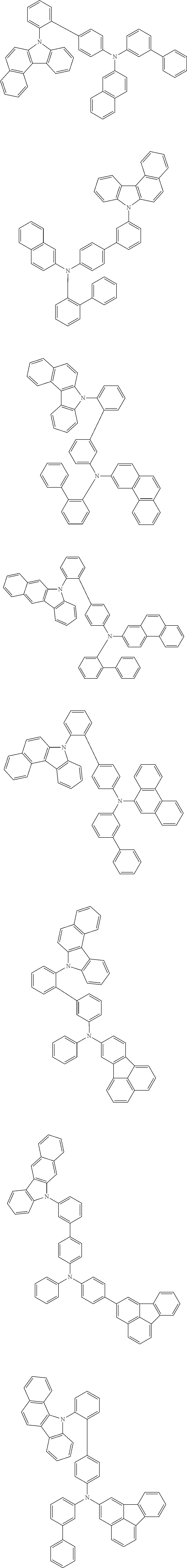 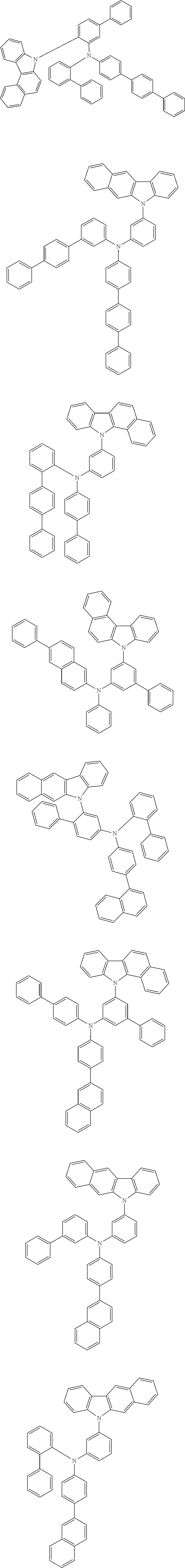 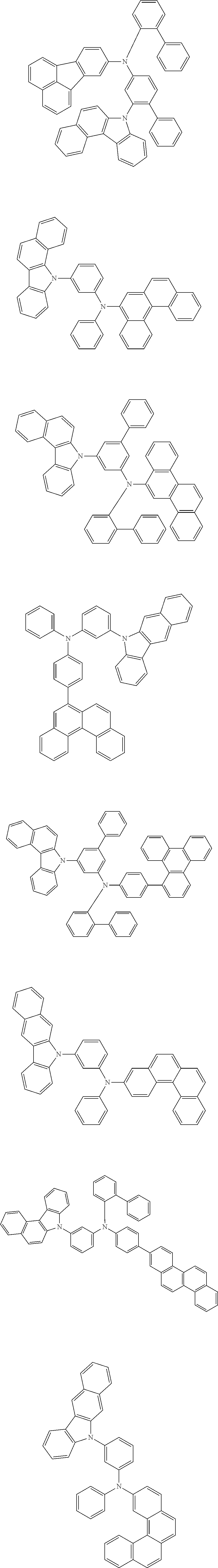 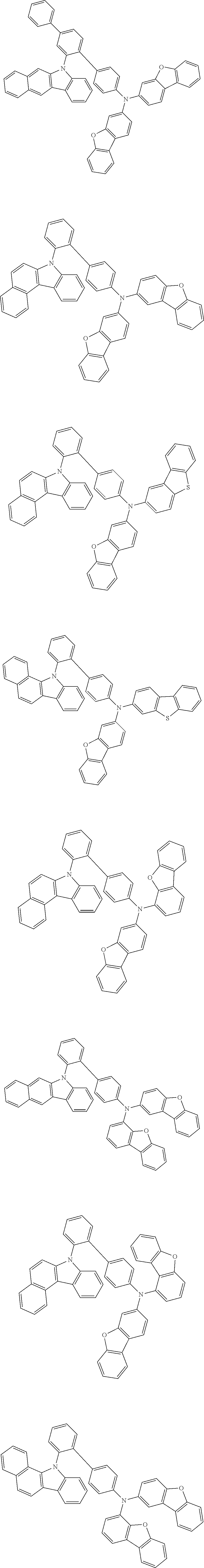 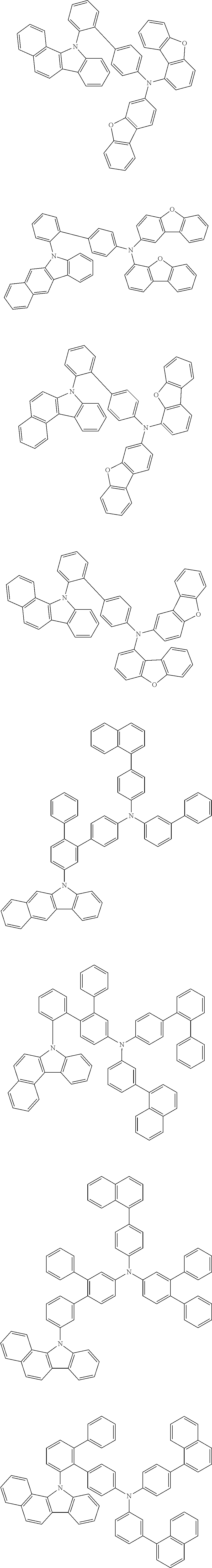 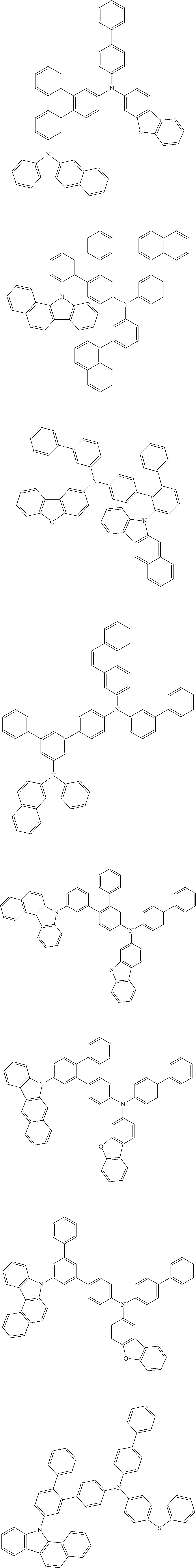 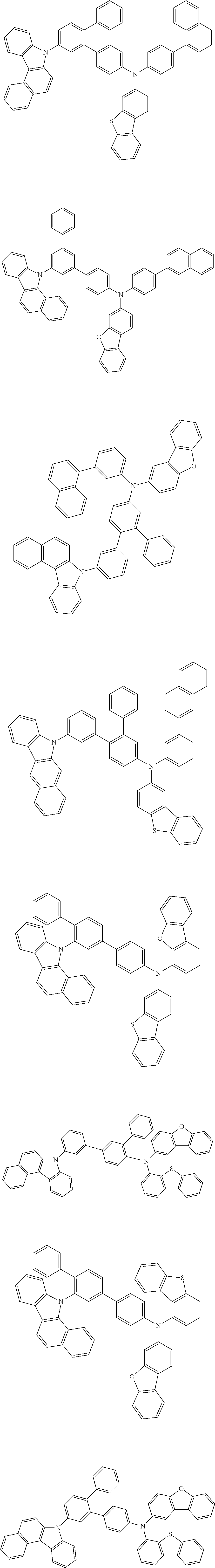 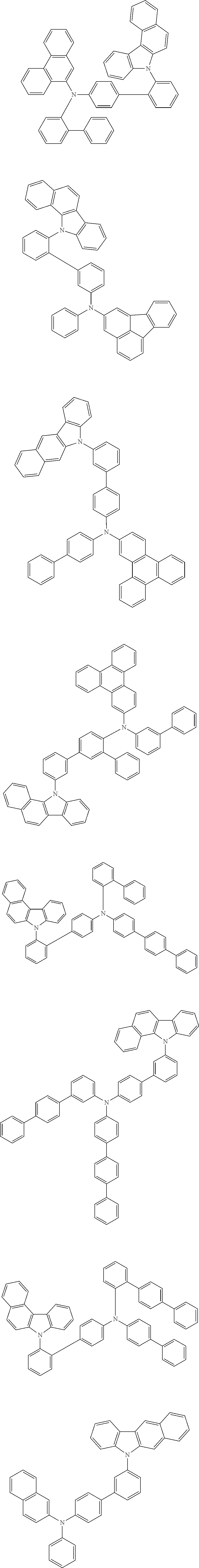 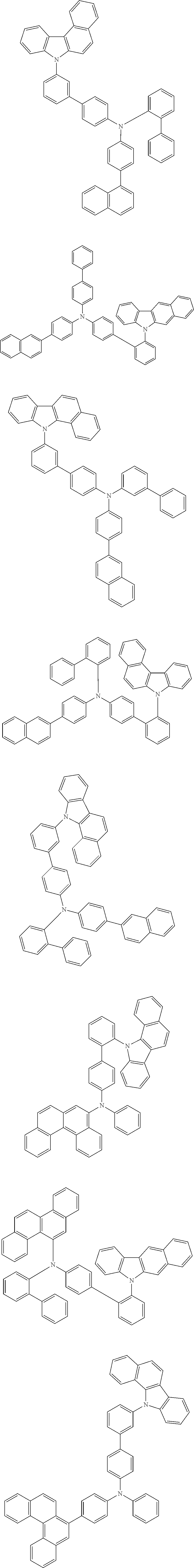 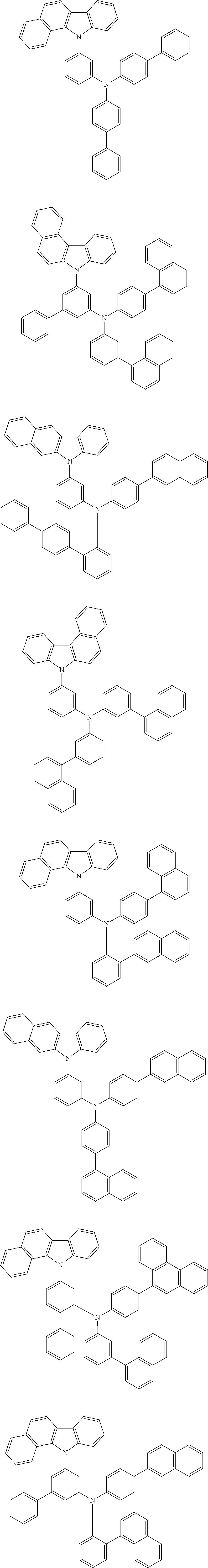 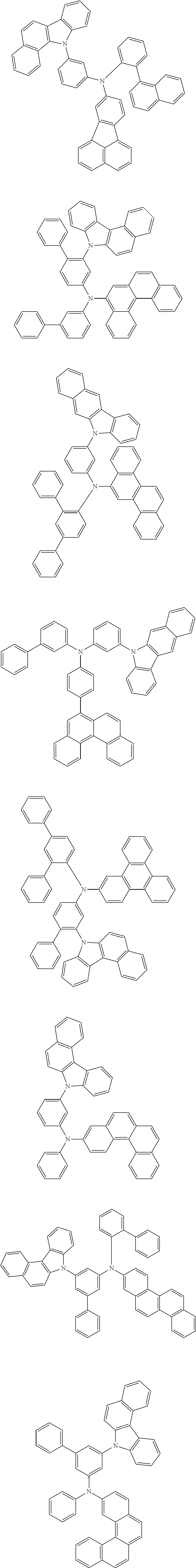 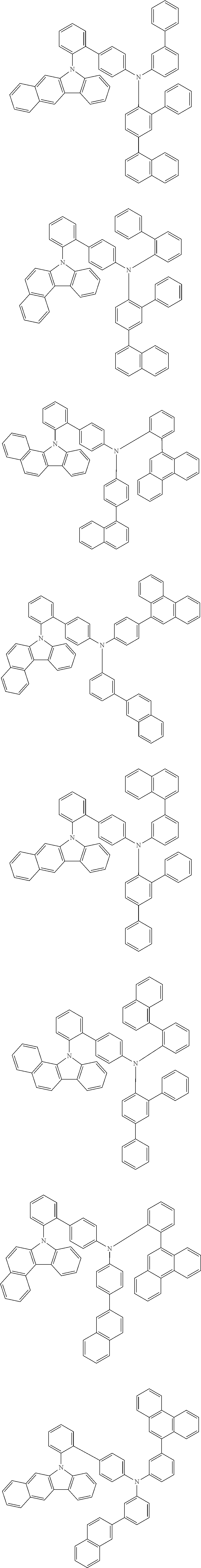 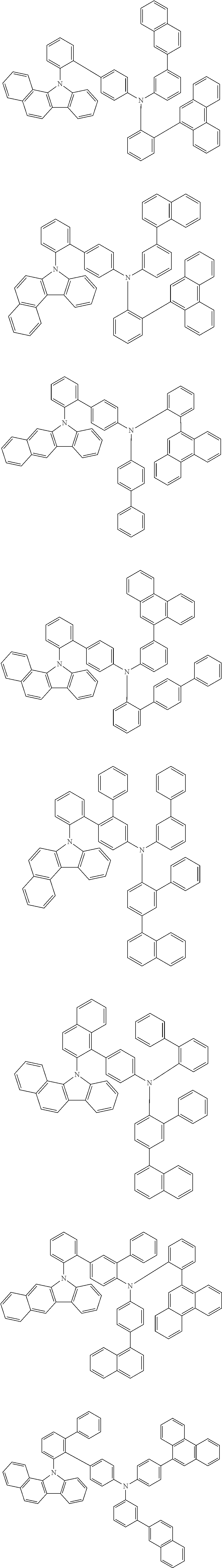 [chemical structure] [chemical structure] 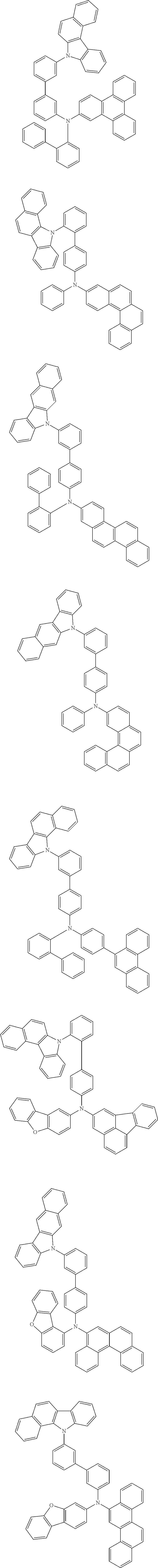 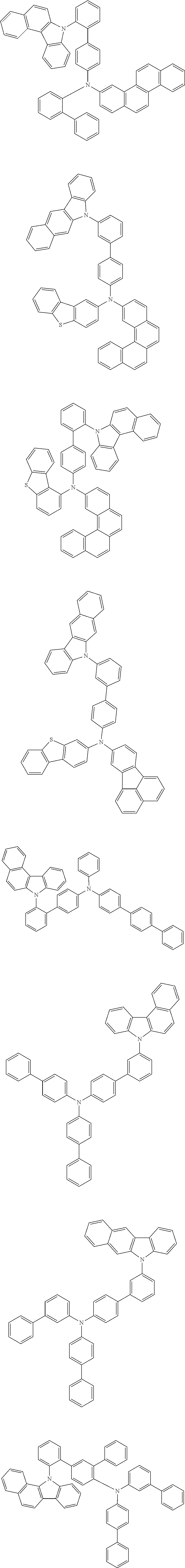 [chemical structure] 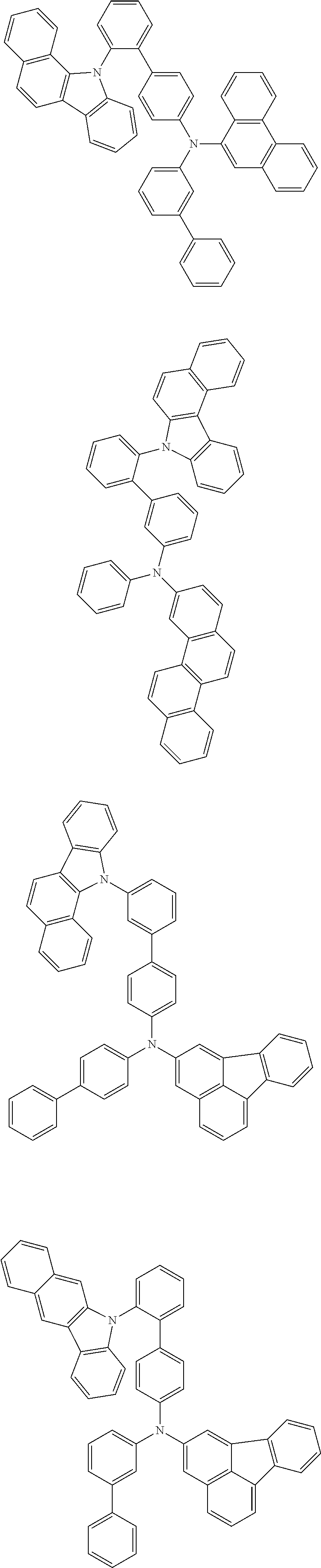 [SMILES: c1ccc(-c2cccc(N(c3ccc(-c4ccccc4-n4c5ccccc5c5ccc6ccccc6c54)cc3)c3cc4ccccc4c4ccccc34)c2)cc1 ; c1ccc(N(c2cccc(-c3ccccc3-n3c4ccccc4c4c5ccccc5ccc43)c2)c2ccc3ccc4c5ccccc5ccc4c3c2)cc1 ; c1ccc(-c2ccc(N(c3ccc(-c4cccc(-n5c6ccccc6c6ccc7ccccc7c65)c4)cc3)c3cc4c5c(cccc5c3)-c3ccccc3-4)cc2)cc1 ; c1ccc(-c2cccc(N(c3ccc(-c4ccccc4-n4c5ccccc5c5cc6ccccc6cc54)cc3)c3cc4c5c(cccc5c3)-c3ccccc3-4)c2)cc1] 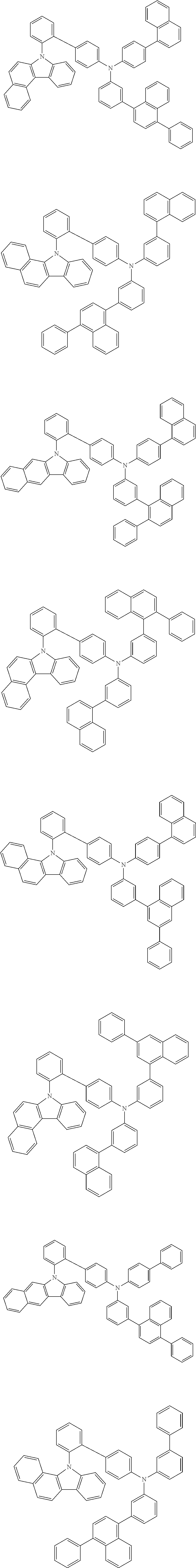 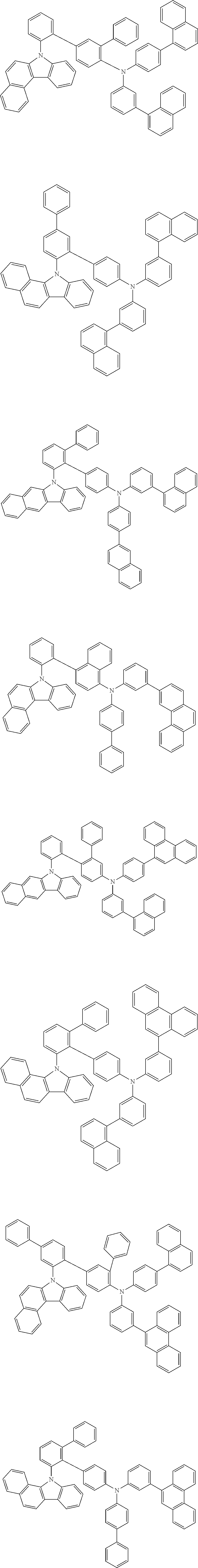 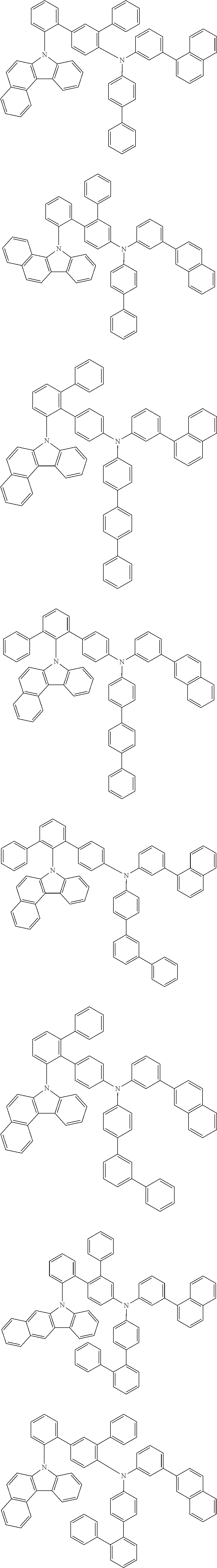 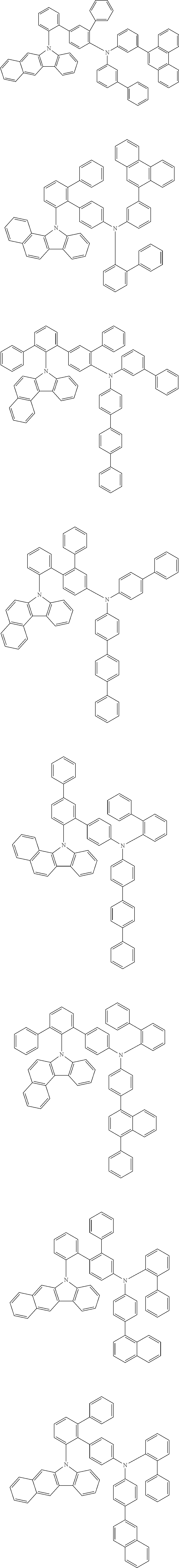 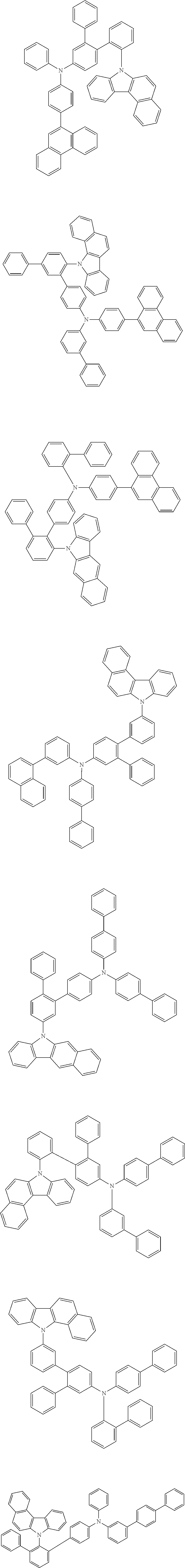 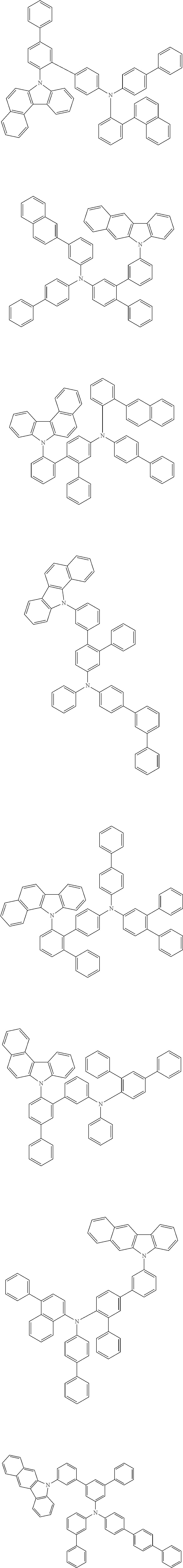 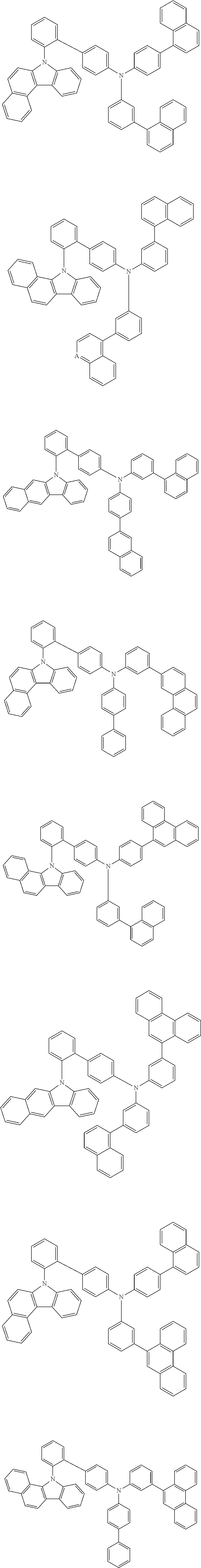 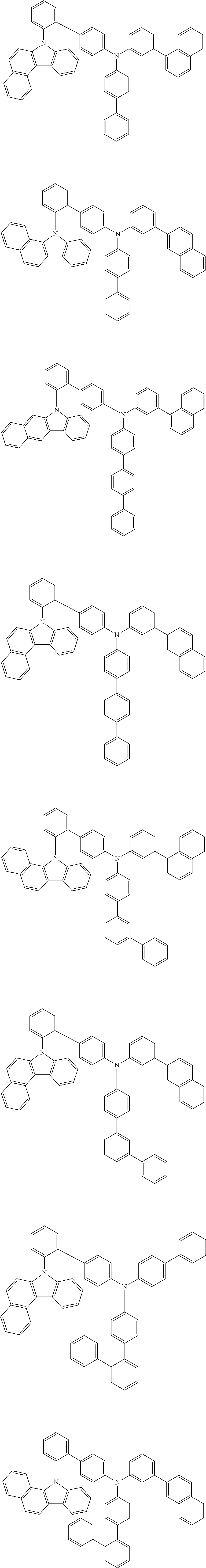 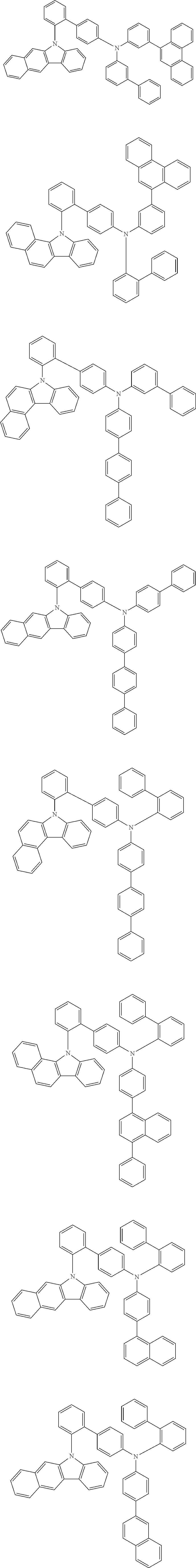 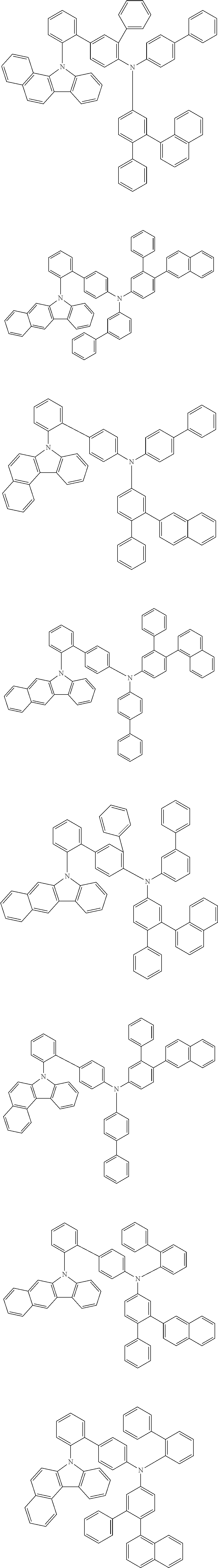 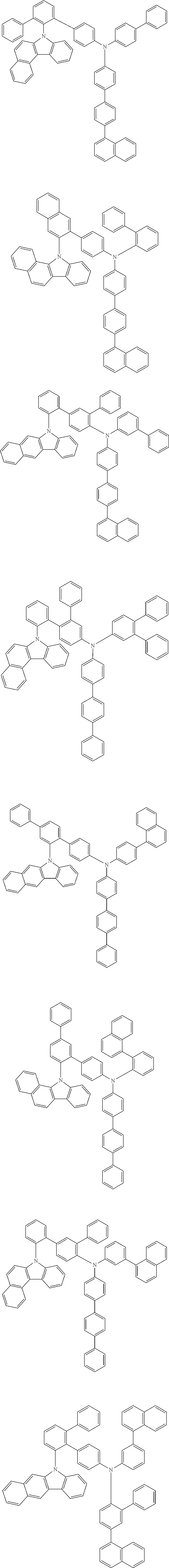 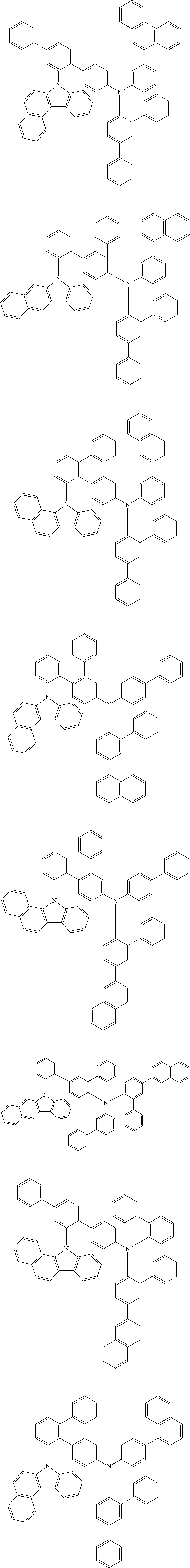 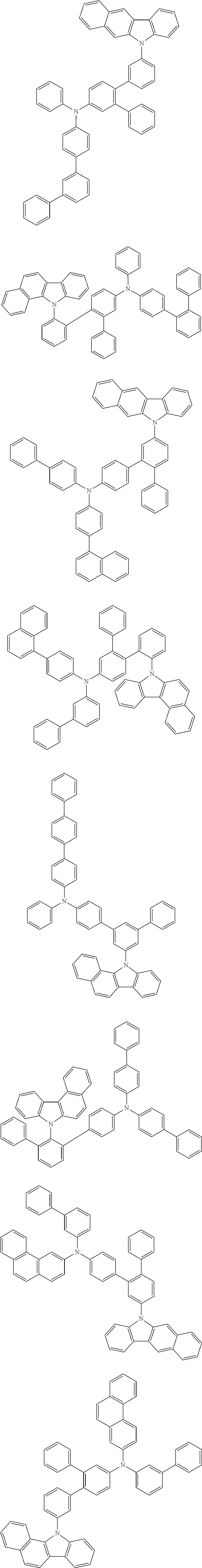 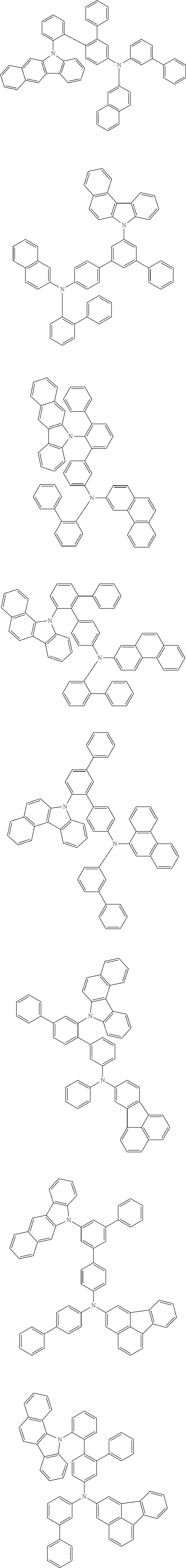 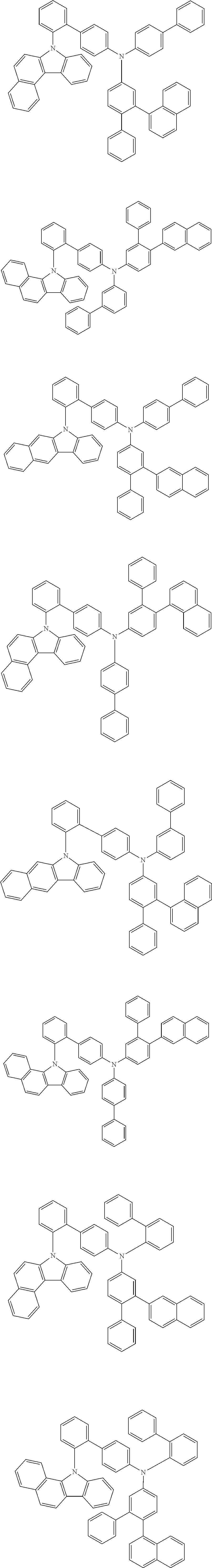 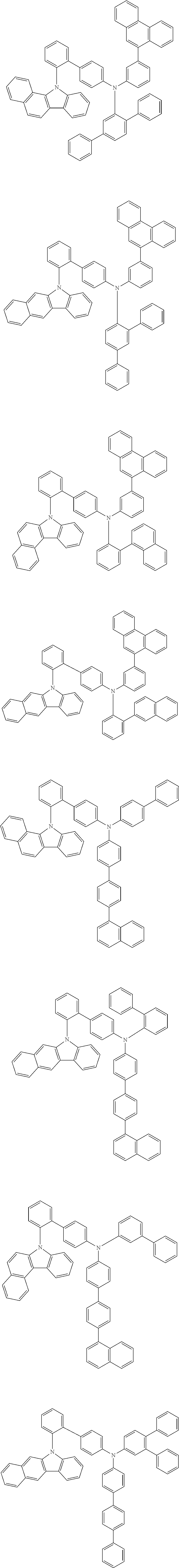 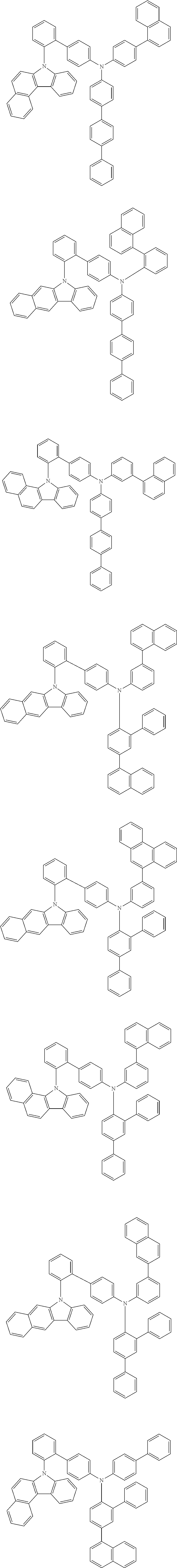 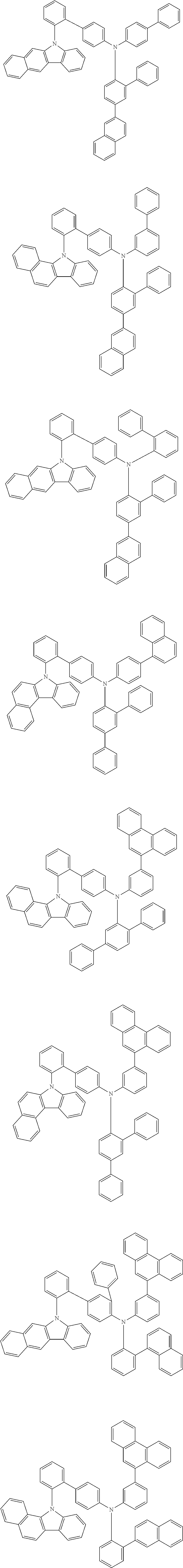 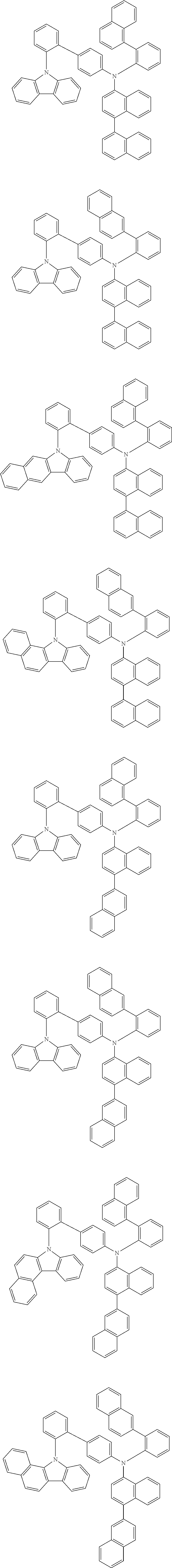 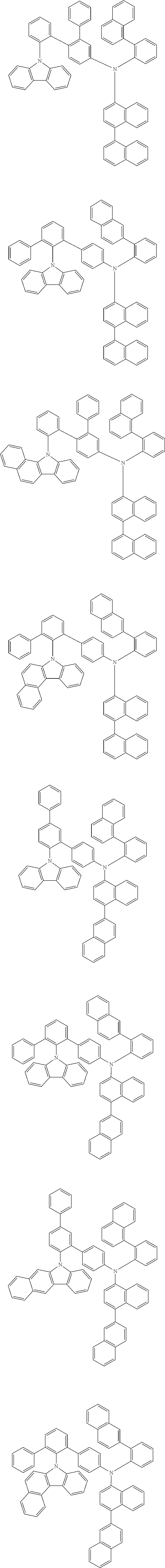 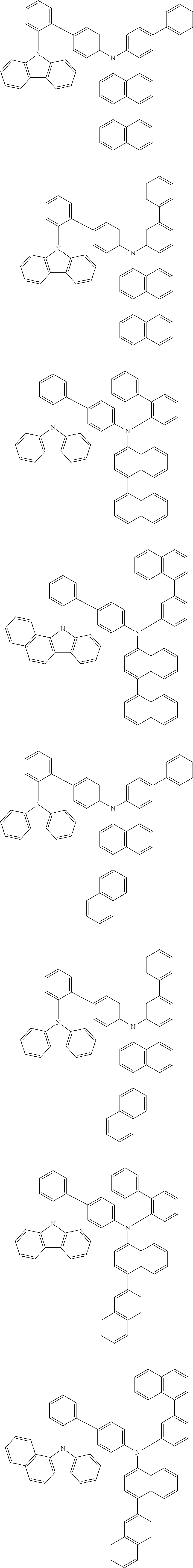 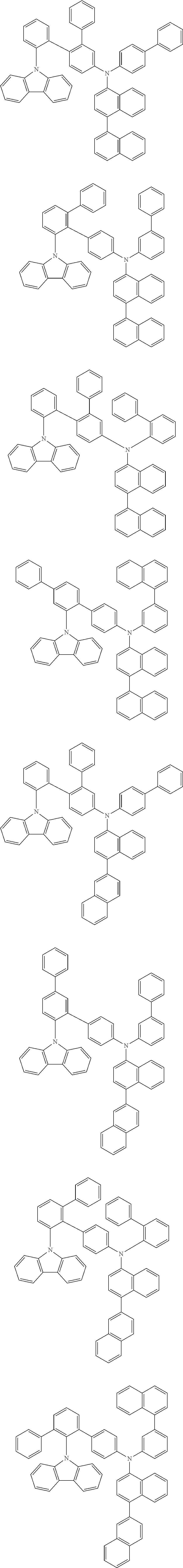 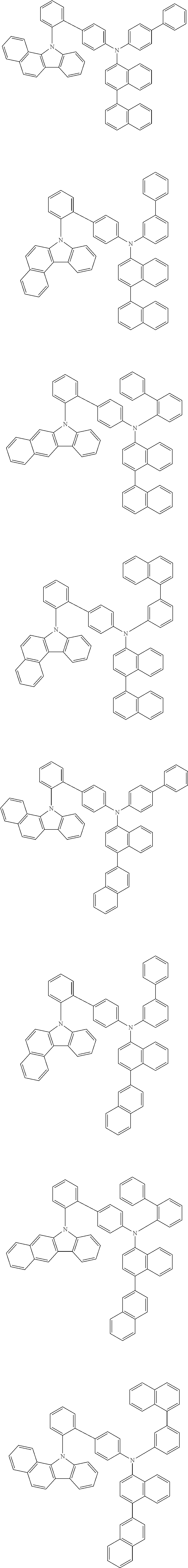 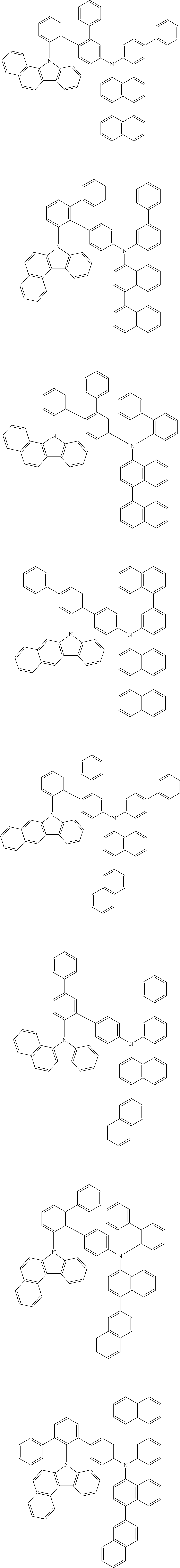 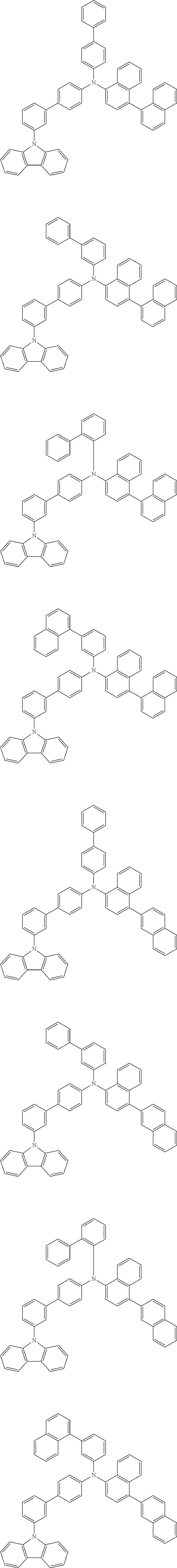 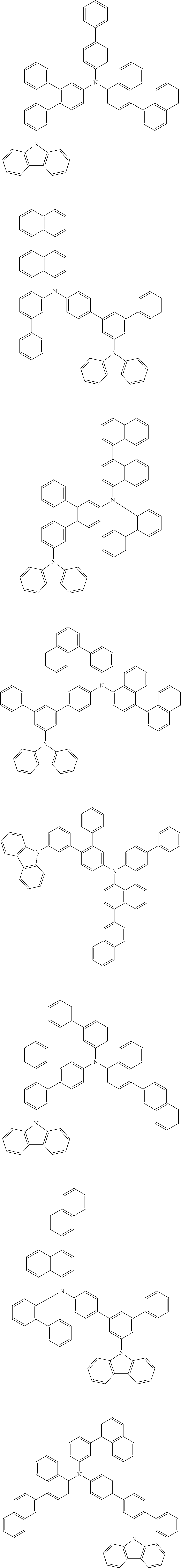 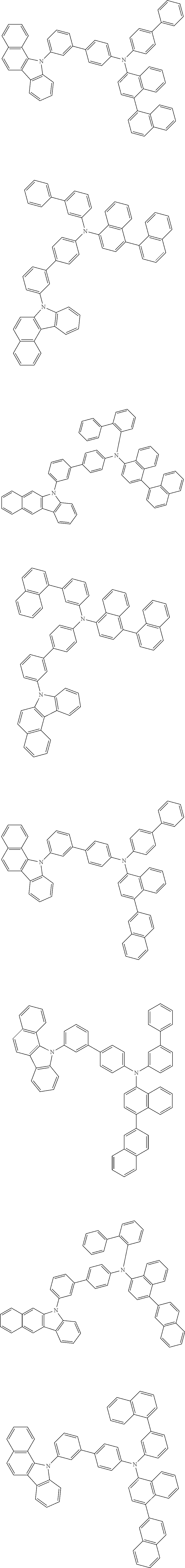 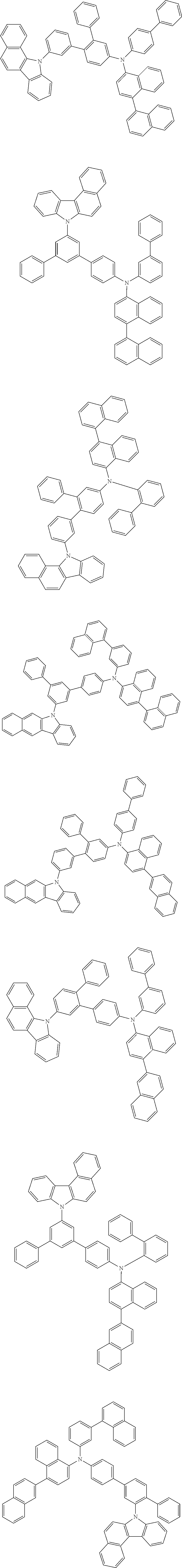 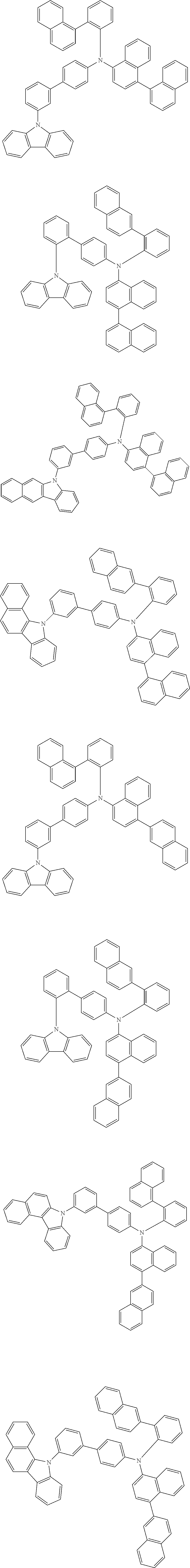 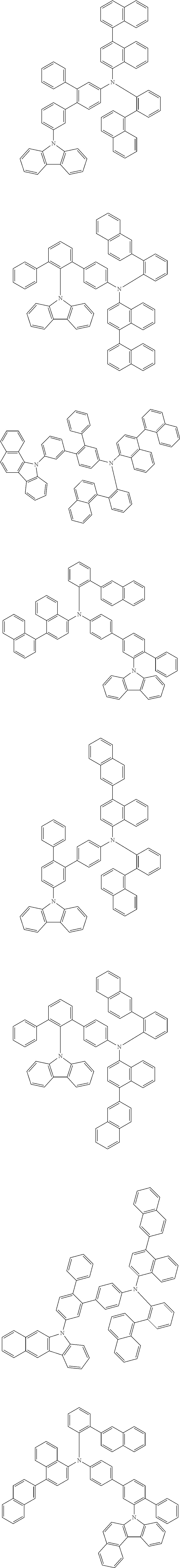 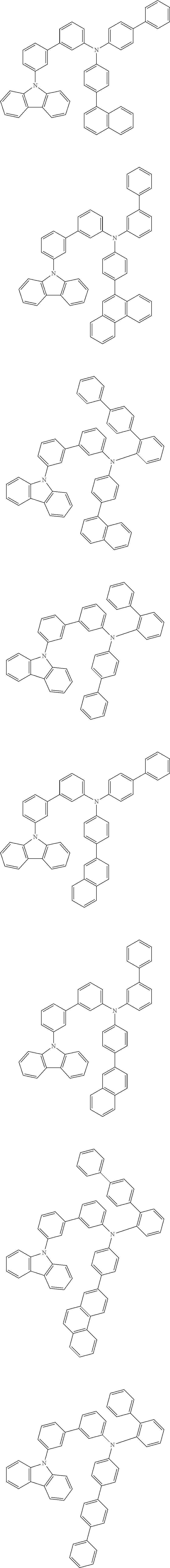 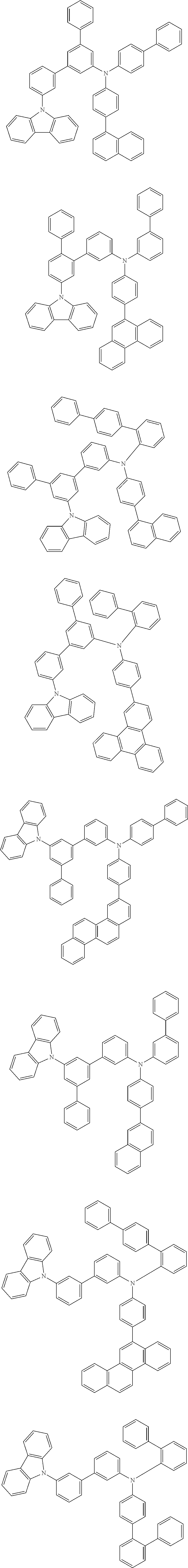 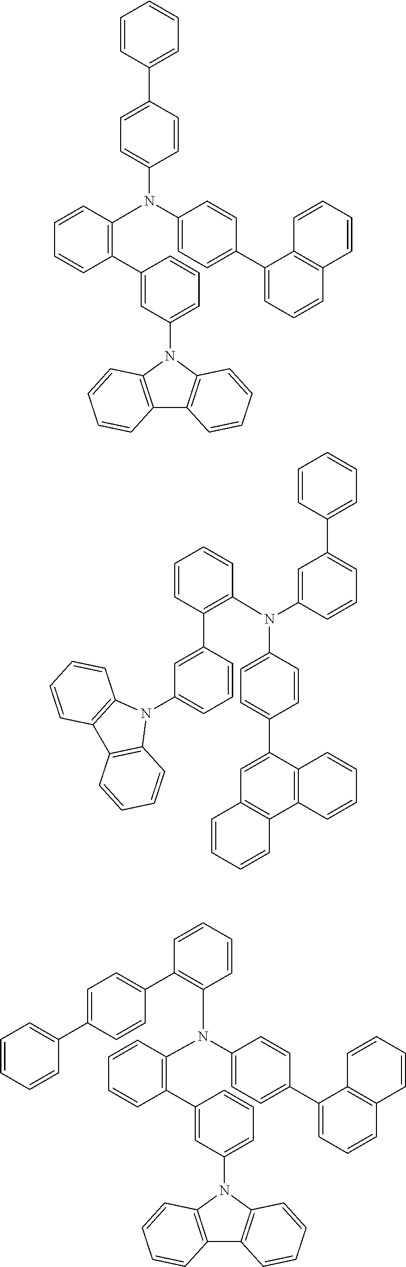 [SMILES: c1ccc(-c2ccc(N(c3ccc(-c4cccc5ccccc45)cc3)c3ccccc3-c3cccc(-n4c5ccccc5c5ccccc54)c3)cc2)cc1 ; c1ccc(-c2cccc(N(c3ccc(-c4cc5ccccc5c5ccccc45)cc3)c3ccccc3-c3cccc(-n4c5ccccc5c5ccccc54)c3)c2)cc1 ; c1ccc(-c2ccc(-c3ccccc3N(c3ccc(-c4cccc5ccccc45)cc3)c3ccccc3-c3cccc(-n4c5ccccc5c5ccccc54)c3)cc2)cc1] 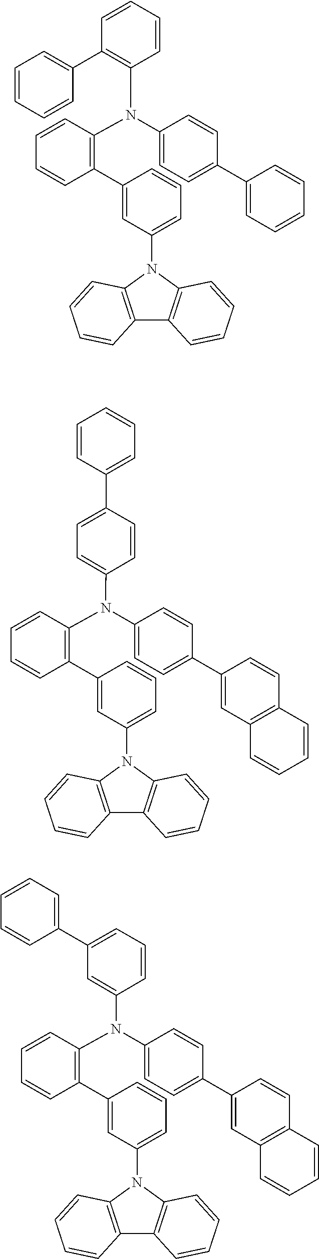 [SMILES: c1ccc(-c2ccc(N(c3ccccc3-c3ccccc3)c3ccccc3-c3cccc(-n4c5ccccc5c5ccccc54)c3)cc2)cc1 ; c1ccc(-c2ccc(N(c3ccc(-c4ccc5ccccc5c4)cc3)c3ccccc3-c3cccc(-n4c5ccccc5c5ccccc54)c3)cc2)cc1 ; c1ccc(-c2cccc(N(c3ccc(-c4ccc5ccccc5c4)cc3)c3ccccc3-c3cccc(-n4c5ccccc5c5ccccc54)c3)c2)cc1] 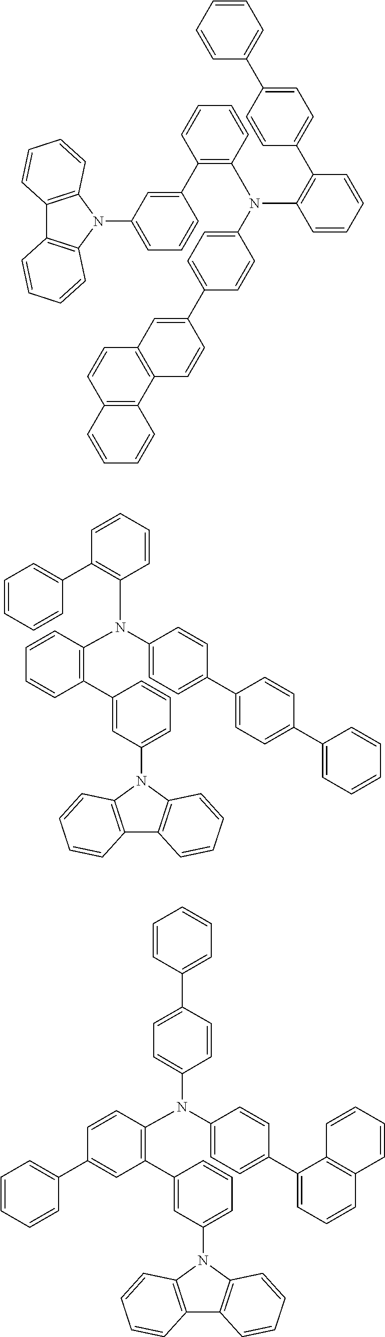 [SMILES: c1ccc(-c2ccc(-c3ccccc3N(c3ccc(-c4ccc5c(ccc6ccccc65)c4)cc3)c3ccccc3-c3cccc(-n4c5ccccc5c5ccccc54)c3)cc2)cc1 ; c1ccc(-c2ccc(-c3ccc(N(c4ccccc4-c4ccccc4)c4ccccc4-c4cccc(-n5c6ccccc6c6ccccc65)c4)cc3)cc2)cc1 ; c1ccc(-c2ccc(N(c3ccc(-c4cccc5ccccc45)cc3)c3ccc(-c4ccccc4)cc3-c3cccc(-n4c5ccccc5c5ccccc54)c3)cc2)cc1] 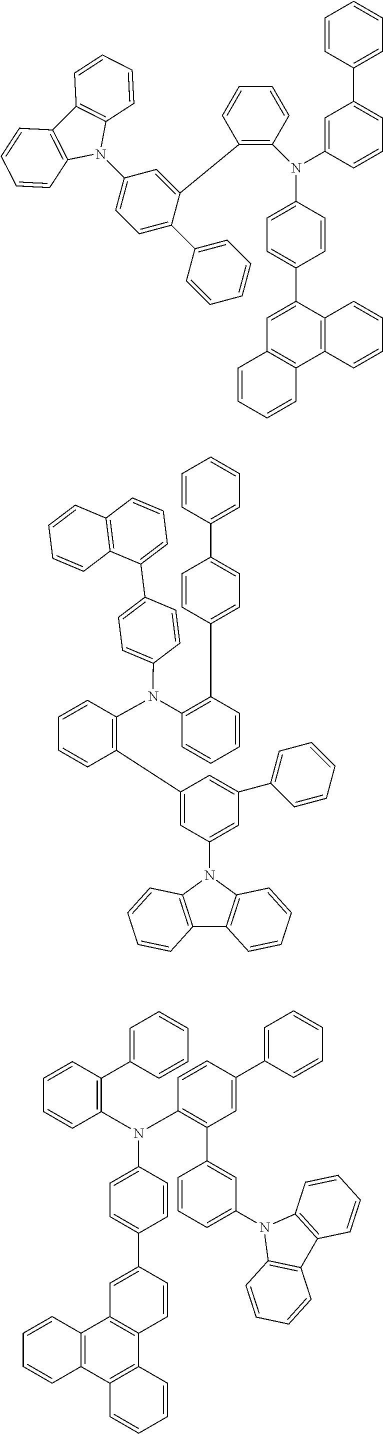 [SMILES: c1ccc(-c2cccc(N(c3ccc(-c4cc5ccccc5c5ccccc45)cc3)c3ccccc3-c3cc(-n4c5ccccc5c5ccccc54)ccc3-c3ccccc3)c2)cc1 ; c1ccc(-c2ccc(-c3ccccc3N(c3ccc(-c4cccc5ccccc45)cc3)c3ccccc3-c3cc(-c4ccccc4)cc(-n4c5ccccc5c5ccccc54)c3)cc2)cc1 ; c1ccc(-c2ccc(N(c3ccc(-c4ccc5c6ccccc6c6ccccc6c5c4)cc3)c3ccccc3-c3ccccc3)c(-c3cccc(-n4c5ccccc5c5ccccc54)c3)c2)cc1] 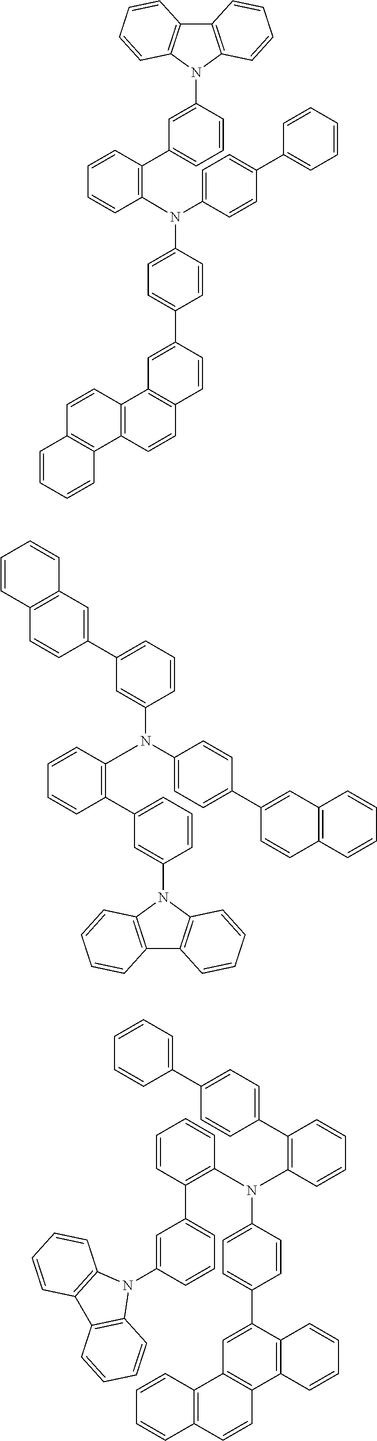 [SMILES: c1ccc(-c2ccc(N(c3ccc(-c4ccc5ccc6c7ccccc7ccc6c5c4)cc3)c3ccccc3-c3cccc(-n4c5ccccc5c5ccccc54)c3)cc2)cc1 ; c1cc(-c2ccc3ccccc3c2)cc(N(c2ccc(-c3ccc4ccccc4c3)cc2)c2ccccc2-c2cccc(-n3c4ccccc4c4ccccc43)c2)c1 ; c1ccc(-c2ccc(-c3ccccc3N(c3ccc(-c4cc5c6ccccc6ccc5c5ccccc45)cc3)c3ccccc3-c3cccc(-n4c5ccccc5c5ccccc54)c3)cc2)cc1] 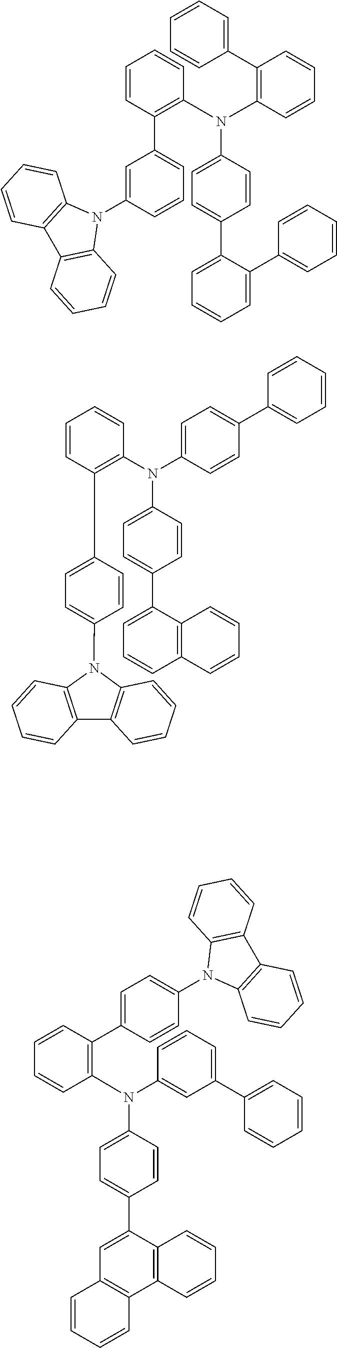 [SMILES: c1ccc(-c2ccccc2-c2ccc(N(c3ccccc3-c3ccccc3)c3ccccc3-c3cccc(-n4c5ccccc5c5ccccc54)c3)cc2)cc1 ; c1ccc(-c2ccc(N(c3ccc(-c4cccc5ccccc45)cc3)c3ccccc3-c3ccc(-n4c5ccccc5c5ccccc54)cc3)cc2)cc1 ; c1ccc(-c2cccc(N(c3ccc(-c4cc5ccccc5c5ccccc45)cc3)c3ccccc3-c3ccc(-n4c5ccccc5c5ccccc54)cc3)c2)cc1] 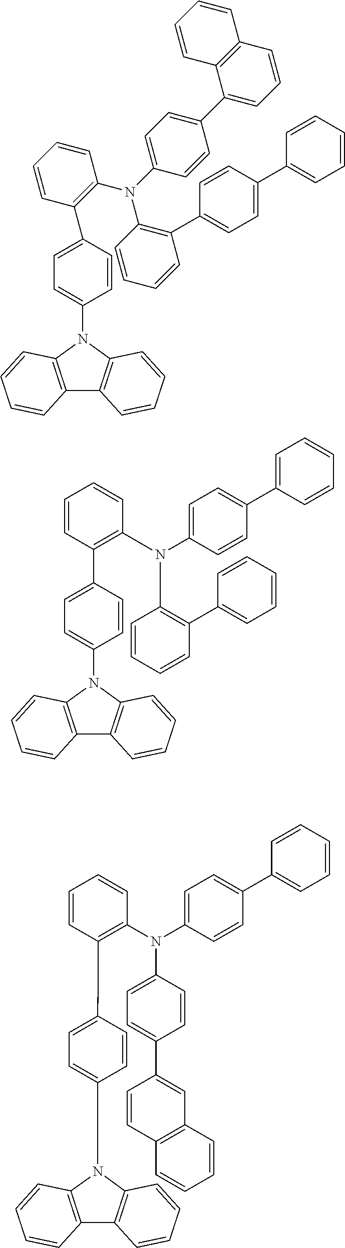 [SMILES: c1ccc(-c2ccc(-c3ccccc3N(c3ccc(-c4cccc5ccccc45)cc3)c3ccccc3-c3ccc(-n4c5ccccc5c5ccccc54)cc3)cc2)cc1 ; c1ccc(-c2ccc(N(c3ccccc3-c3ccccc3)c3ccccc3-c3ccc(-n4c5ccccc5c5ccccc54)cc3)cc2)cc1 ; c1ccc(-c2ccc(N(c3ccc(-c4ccc5ccccc5c4)cc3)c3ccccc3-c3ccc(-n4c5ccccc5c5ccccc54)cc3)cc2)cc1] 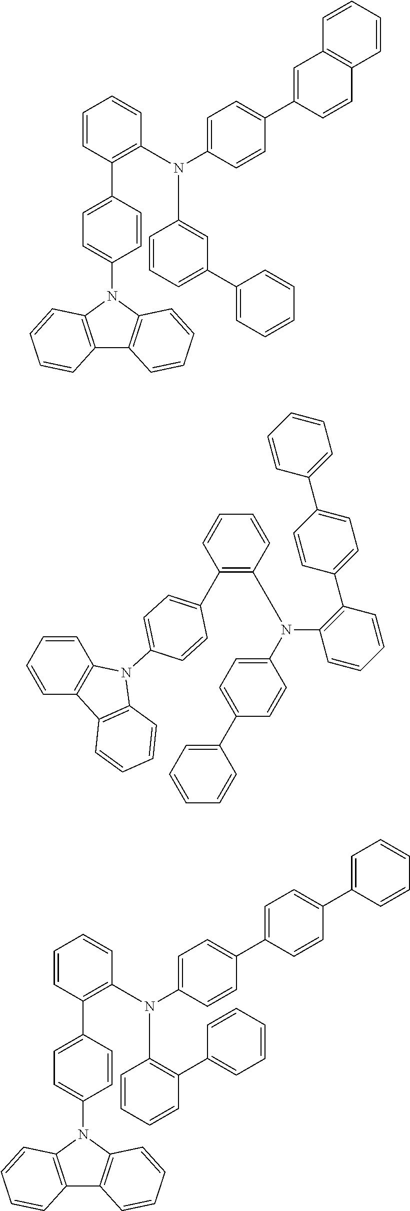 [SMILES: c1ccc(-c2cccc(N(c3ccc(-c4ccc5ccccc5c4)cc3)c3ccccc3-c3ccc(-n4c5ccccc5c5ccccc54)cc3)c2)cc1 ; c1ccc(-c2ccc(-c3ccccc3N(Cc3ccccc3-c3ccc(-n4c5ccccc5c5ccccc54)cc3)c3ccc(-c4ccccc4)cc3)cc2)cc1 ; c1ccc(-c2ccc(-c3ccc(N(c4ccccc4-c4ccccc4)c4ccccc4-c4ccc(-n5c6ccccc6c6ccccc65)cc4)cc3)cc2)cc1] 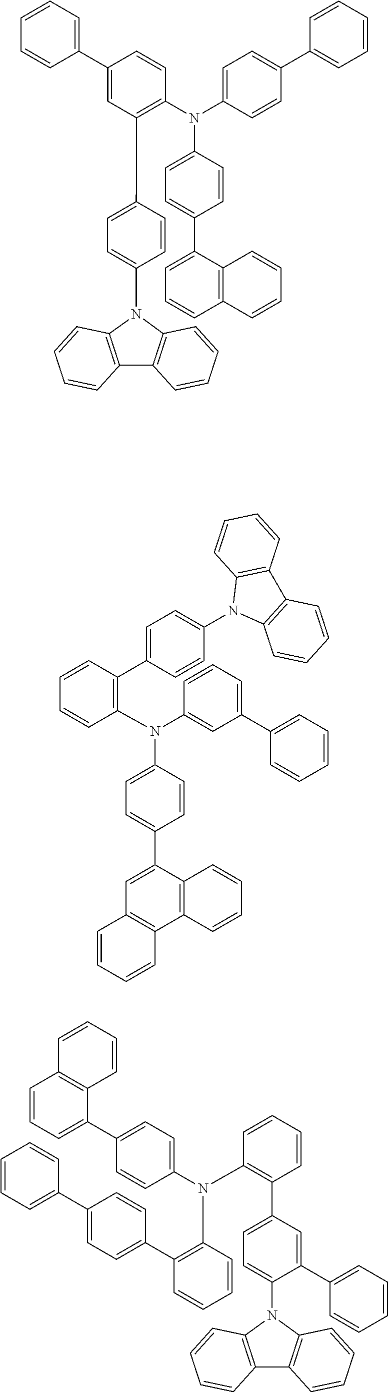 [SMILES: c1ccc(-c2ccc(N(c3ccc(-c4cccc5ccccc45)cc3)c3ccc(-c4ccccc4)cc3-c3ccc(-n4c5ccccc5c5ccccc54)cc3)cc2)cc1 ; c1ccc(-c2cccc(N(c3ccc(-c4cc5ccccc5c5ccccc45)cc3)c3ccccc3-c3ccc(-n4c5ccccc5c5ccccc54)cc3)c2)cc1 ; c1ccc(-c2ccc(-c3ccccc3N(c3ccc(-c4cccc5ccccc45)cc3)c3ccccc3-c3ccc(-n4c5ccccc5c5ccccc54)c(-c4ccccc4)c3)cc2)cc1] 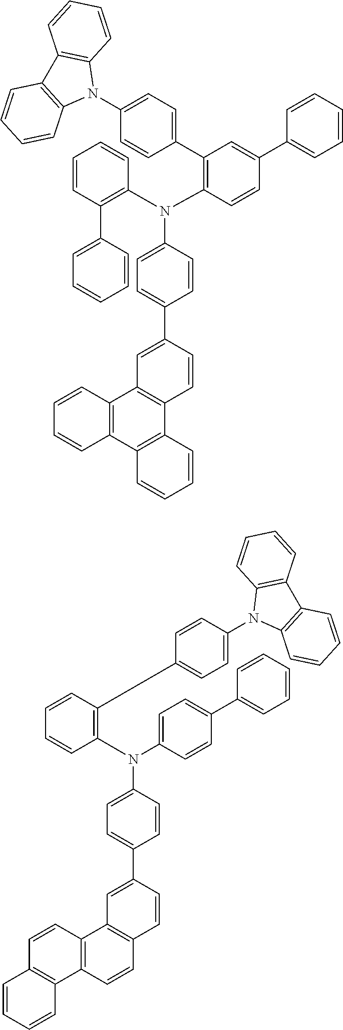 [SMILES: c1ccc(-c2ccc(N(c3ccc(-c4ccc5c6ccccc6c6ccccc6c5c4)cc3)c3ccccc3-c3ccccc3)c(-c3ccc(-n4c5ccccc5c5ccccc54)cc3)c2)cc1 ; c1ccc(-c2ccc(N(c3ccc(-c4ccc5ccc6c7ccccc7ccc6c5c4)cc3)c3ccccc3-c3ccc(-n4c5ccccc5c5ccccc54)cc3)cc2)cc1] 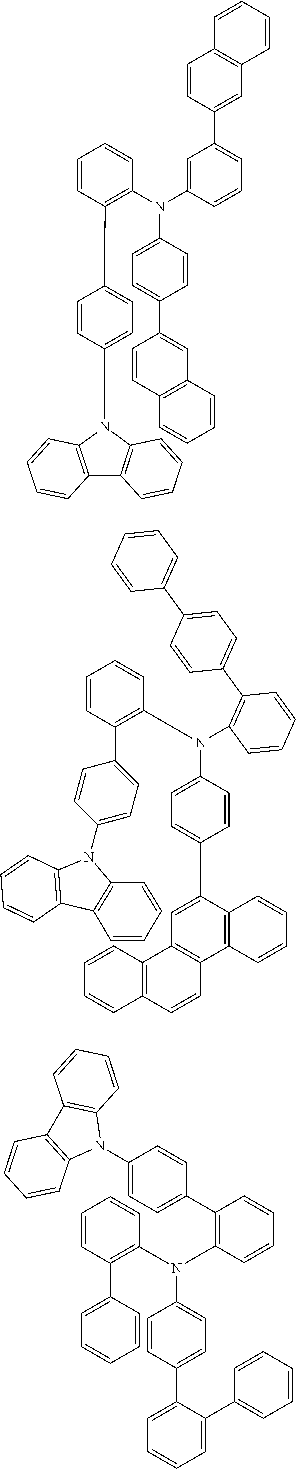 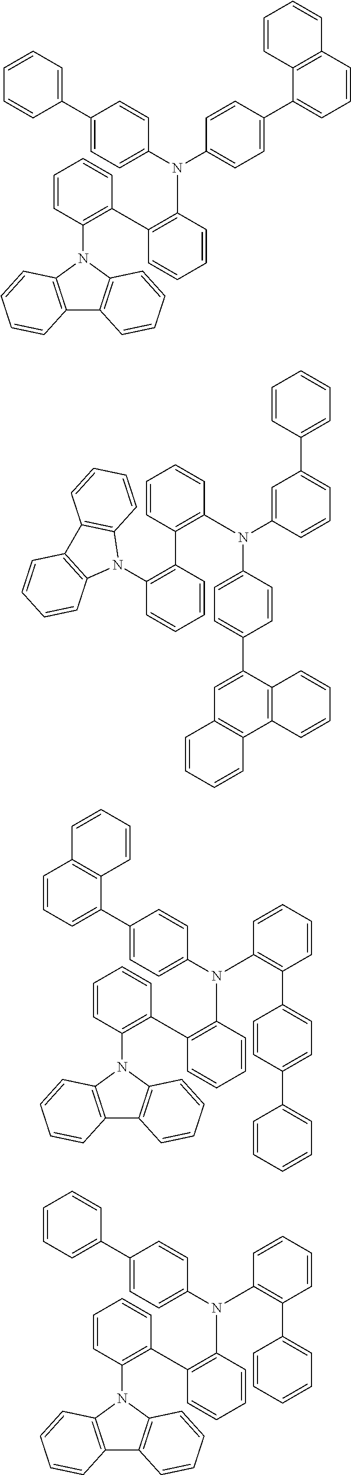 [SMILES: c1ccc(-c2ccc(N(c3ccc(-c4cccc5ccccc45)cc3)c3ccccc3-c3ccccc3-n3c4ccccc4c4ccccc43)cc2)cc1 ; c1ccc(-c2cccc(N(c3ccc(-c4cc5ccccc5c5ccccc45)cc3)c3ccccc3-c3ccccc3-n3c4ccccc4c4ccccc43)c2)cc1 ; c1ccc(-c2ccc(-c3ccccc3N(c3ccc(-c4cccc5ccccc45)cc3)c3ccccc3-c3ccccc3-n3c4ccccc4c4ccccc43)cc2)cc1 ; c1ccc(-c2ccc(N(c3ccccc3-c3ccccc3)c3ccccc3-c3ccccc3-n3c4ccccc4c4ccccc43)cc2)cc1] 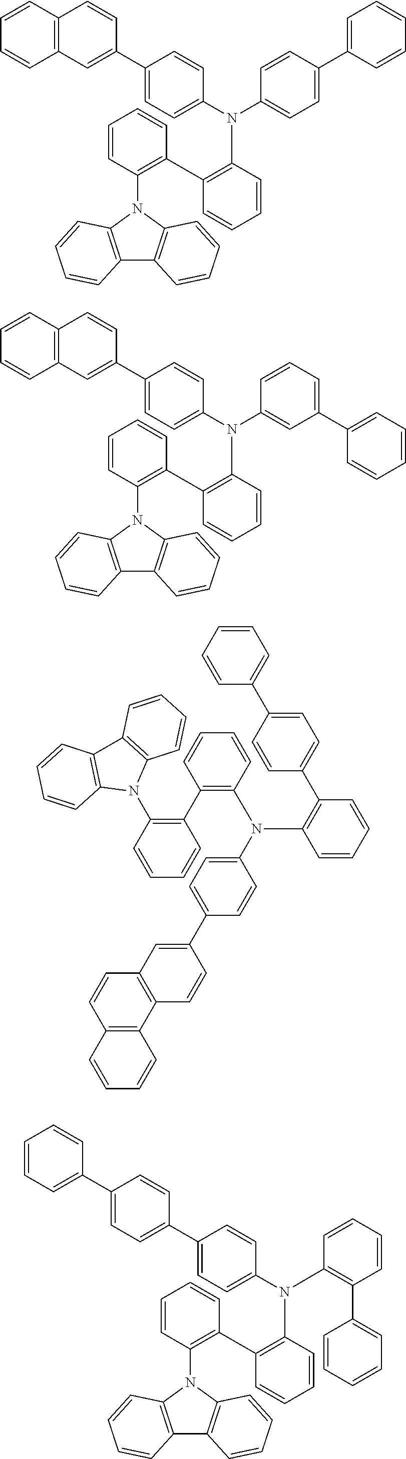 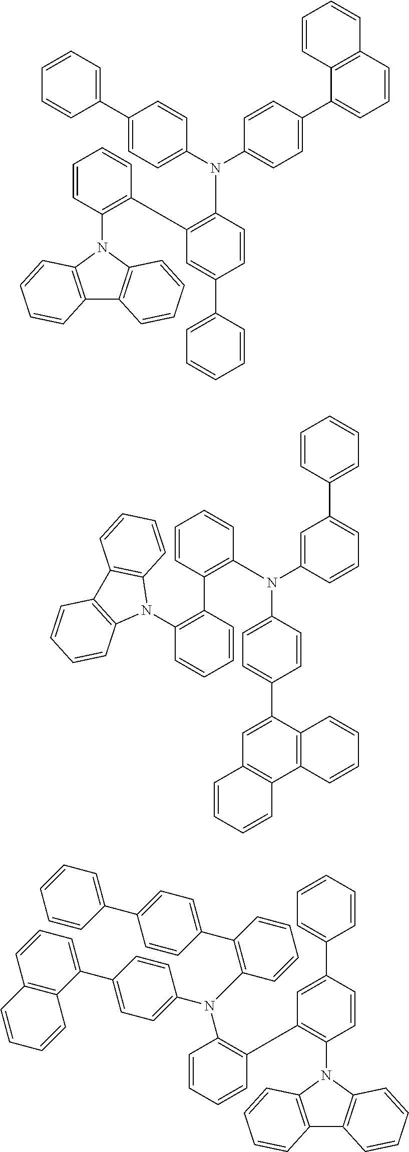 [SMILES: c1ccc(-c2ccc(N(c3ccc(-c4cccc5ccccc45)cc3)c3ccc(-c4ccccc4)cc3-c3ccccc3-n3c4ccccc4c4ccccc43)cc2)cc1 ; c1ccc(-c2cccc(N(c3ccc(-c4cc5ccccc5c5ccccc45)cc3)c3ccccc3-c3ccccc3-n3c4ccccc4c4ccccc43)c2)cc1 ; c1ccc(-c2ccc(-c3ccccc3N(c3ccc(-c4cccc5ccccc45)cc3)c3ccccc3-c3cc(-c4ccccc4)ccc3-n3c4ccccc4c4ccccc43)cc2)cc1] 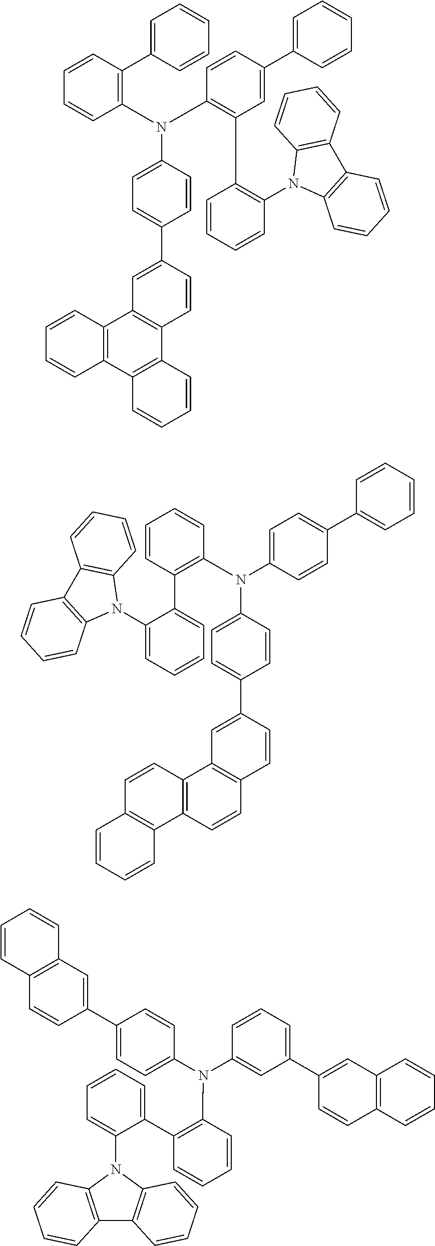 [SMILES: c1ccc(-c2ccc(N(c3ccc(-c4ccc5c6ccccc6c6ccccc6c5c4)cc3)c3ccccc3-c3ccccc3)c(-c3ccccc3-n3c4ccccc4c4ccccc43)c2)cc1 ; c1ccc(-c2ccc(N(c3ccc(-c4ccc5ccc6c7ccccc7ccc6c5c4)cc3)c3ccccc3-c3ccccc3-n3c4ccccc4c4ccccc43)cc2)cc1 ; c1cc(-c2ccc3ccccc3c2)cc(N(c2ccc(-c3ccc4ccccc4c3)cc2)c2ccccc2-c2ccccc2-n2c3ccccc3c3ccccc32)c1] 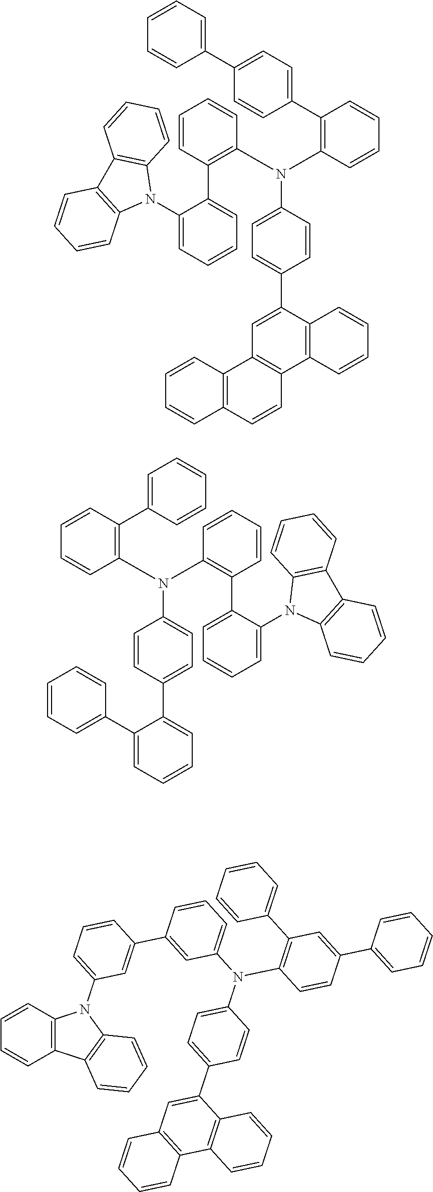 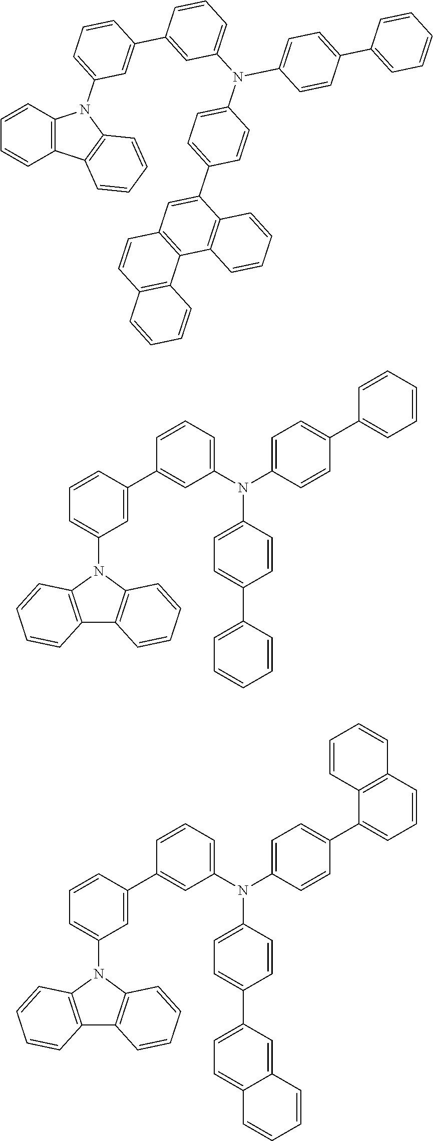 [SMILES: c1ccc(-c2ccc(N(c3ccc(-c4cc5ccc6ccccc6c5c5ccccc45)cc3)c3cccc(-c4cccc(-n5c6ccccc6c6ccccc65)c4)c3)cc2)cc1 ; c1ccc(-c2ccc(N(c3ccc(-c4ccccc4)cc3)c3cccc(-c4cccc(-n5c6ccccc6c6ccccc65)c4)c3)cc2)cc1 ; c1cc(-c2cccc(-n3c4ccccc4c4ccccc43)c2)cc(N(c2ccc(-c3ccc4ccccc4c3)cc2)c2ccc(-c3cccc4ccccc34)cc2)c1] 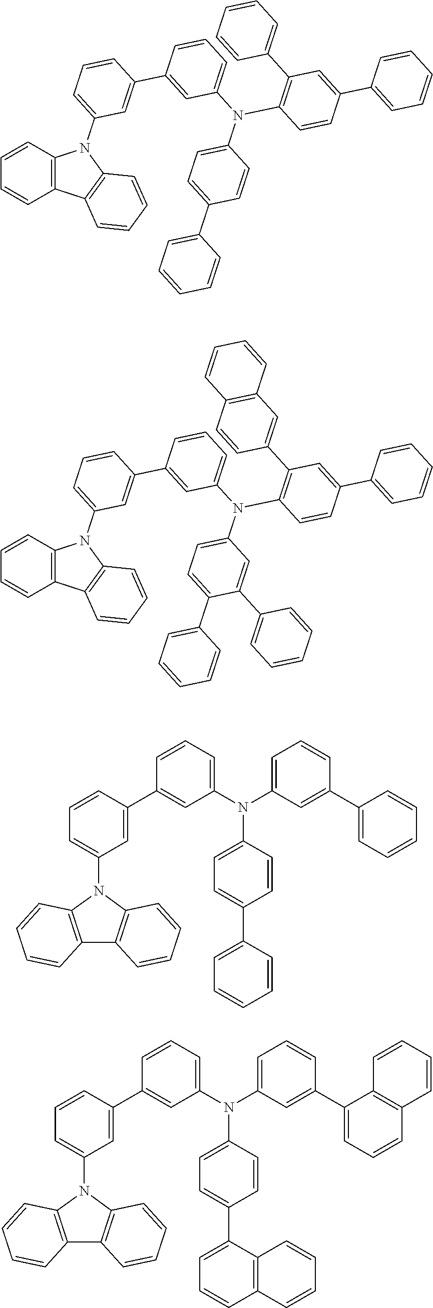 [SMILES: c1ccc(-c2ccc(N(c3cccc(-c4cccc(-n5c6ccccc6c6ccccc65)c4)c3)c3ccc(-c4ccccc4)cc3-c3ccccc3)cc2)cc1 ; c1ccc(-c2ccc(N(c3cccc(-c4cccc(-n5c6ccccc6c6ccccc65)c4)c3)c3ccc(-c4ccccc4)c(-c4ccccc4)c3)c(-c3ccc4ccccc4c3)c2)cc1 ; c1ccc(-c2ccc(N(c3cccc(-c4ccccc4)c3)c3cccc(-c4cccc(-n5c6ccccc6c6ccccc65)c4)c3)cc2)cc1 ; c1cc(-c2cccc(-n3c4ccccc4c4ccccc43)c2)cc(N(c2ccc(-c3cccc4ccccc34)cc2)c2cccc(-c3cccc4ccccc34)c2)c1] 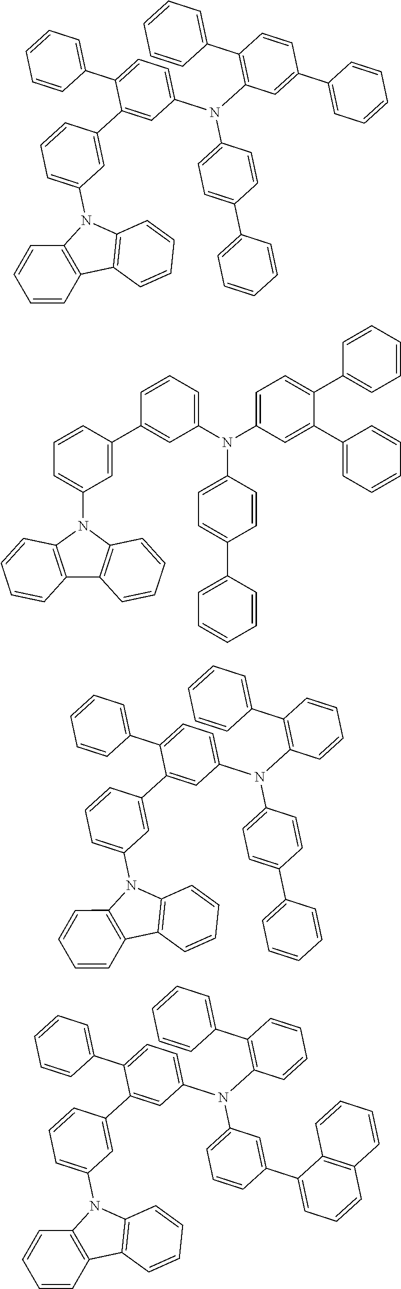 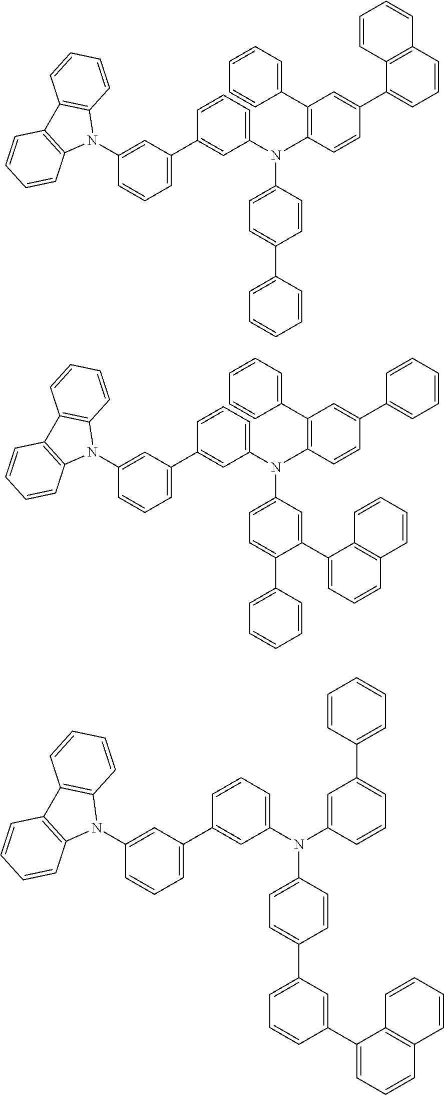 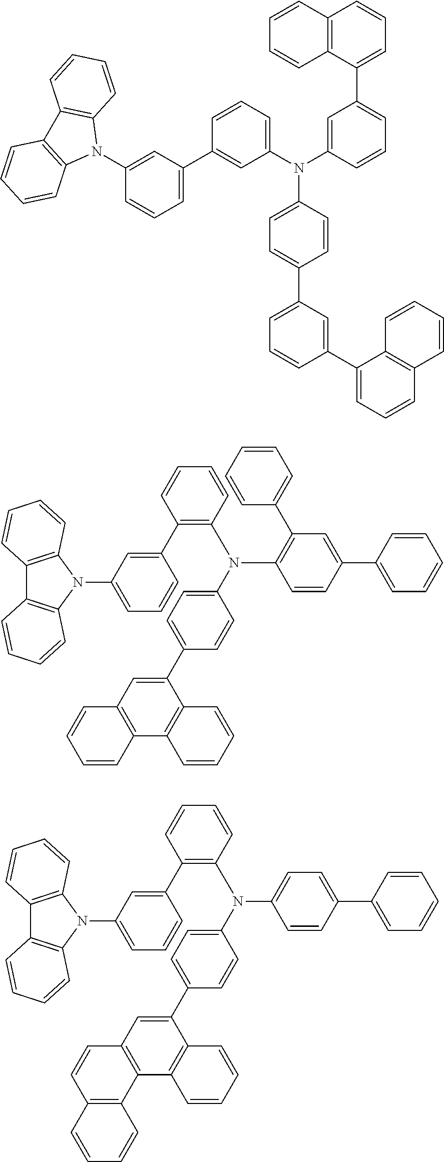 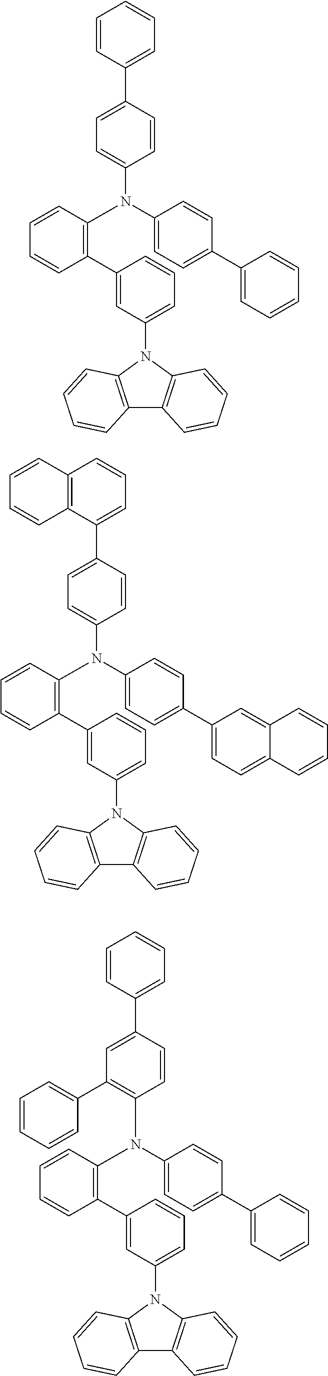 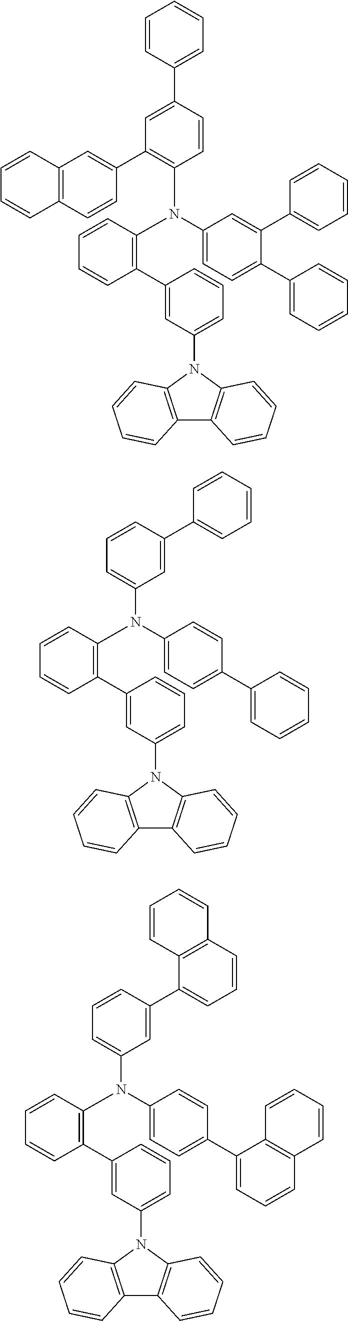 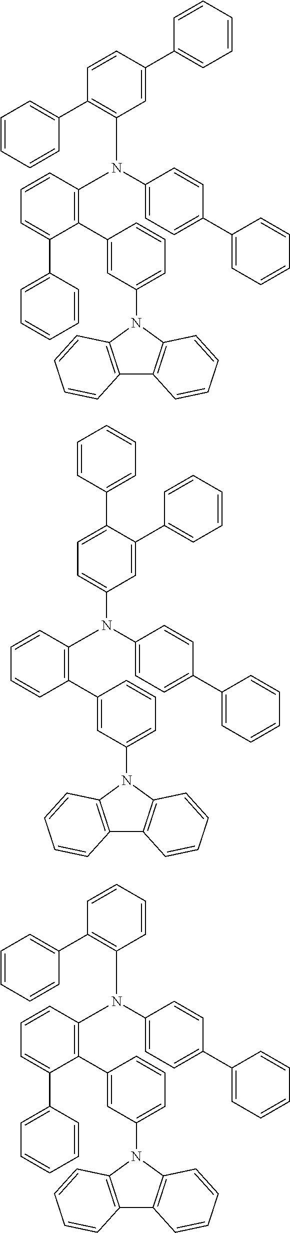 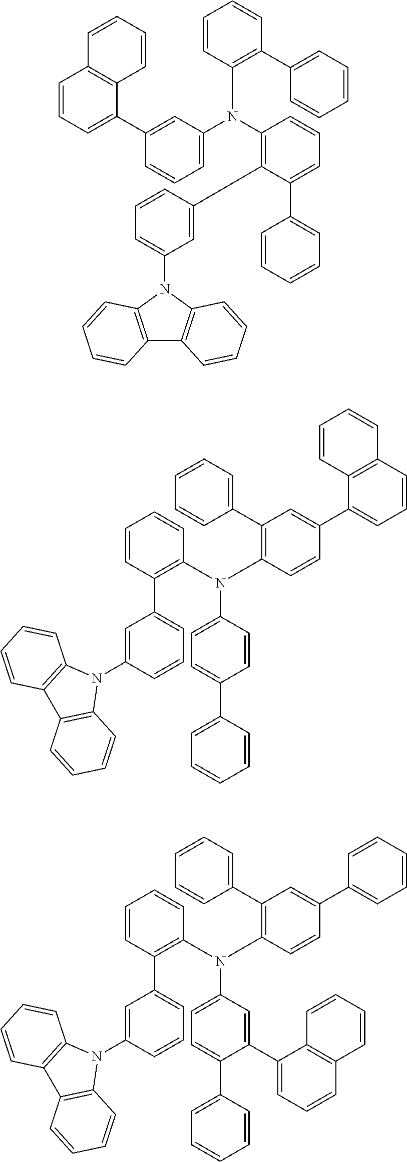 [SMILES: c1ccc(-c2ccccc2N(c2cccc(-c3cccc4ccccc34)c2)c2cccc(-c3ccccc3)c2-c2cccc(-n3c4ccccc4c4ccccc43)c2)cc1 ; c1ccc(-c2ccc(N(c3ccccc3-c3cccc(-n4c5ccccc5c5ccccc54)c3)c3ccc(-c4cccc5ccccc45)cc3-c3ccccc3)cc2)cc1 ; c1ccc(-c2ccc(N(c3ccc(-c4ccccc4)c(-c4cccc5ccccc45)c3)c3ccccc3-c3cccc(-n4c5ccccc5c5ccccc54)c3)c(-c3ccccc3)c2)cc1] 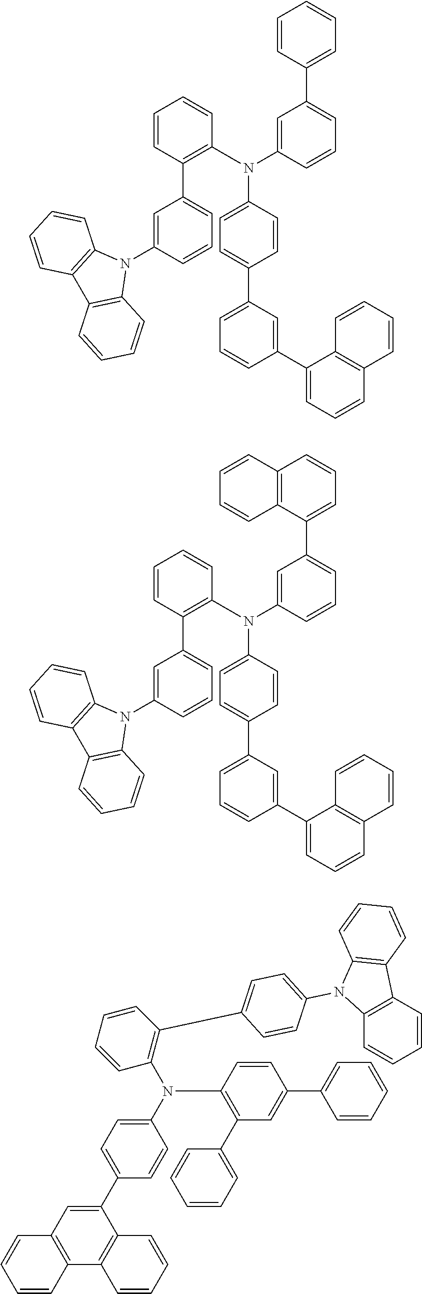 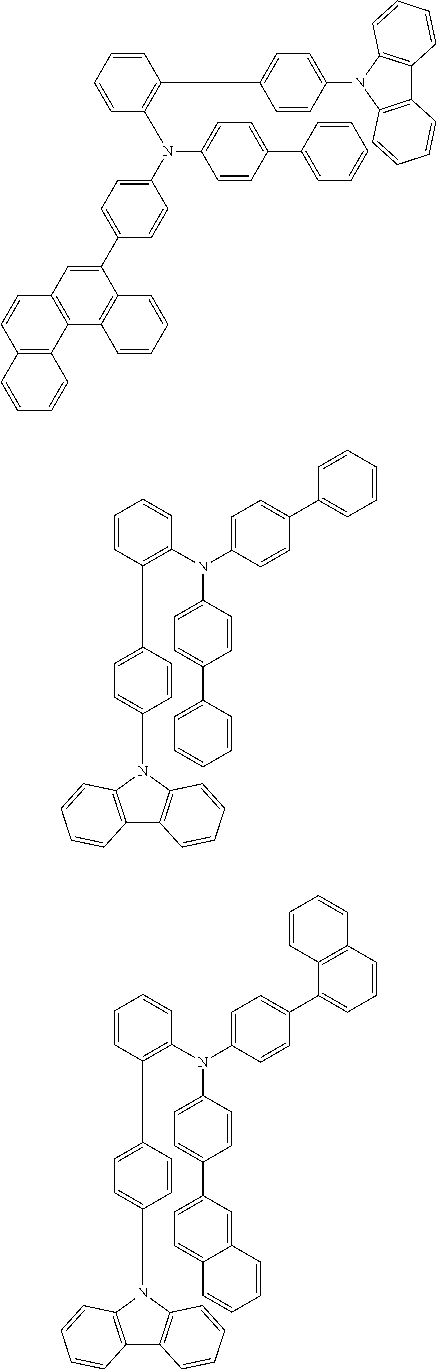 [SMILES: c1ccc(-c2ccc(N(c3ccc(-c4cc5ccc6ccccc6c5c5ccccc45)cc3)c3ccccc3-c3ccc(-n4c5ccccc5c5ccccc54)cc3)cc2)cc1 ; c1ccc(-c2ccc(N(c3ccc(-c4ccccc4)cc3)c3ccccc3-c3ccc(-n4c5ccccc5c5ccccc54)cc3)cc2)cc1 ; c1ccc(N(c2ccc(-c3ccc4ccccc4c3)cc2)c2ccc(-c3cccc4ccccc34)cc2)c(-c2ccc(-n3c4ccccc4c4ccccc43)cc2)c1] 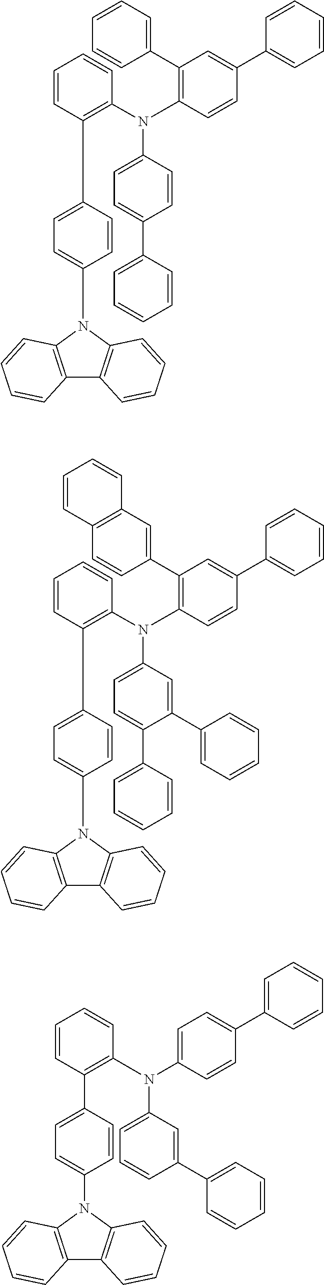 [SMILES: c1ccc(-c2ccc(N(c3ccccc3-c3ccc(-n4c5ccccc5c5ccccc54)cc3)c3ccc(-c4ccccc4)cc3-c3ccccc3)cc2)cc1 ; c1ccc(-c2ccc(N(c3ccc(-c4ccccc4)c(-c4ccccc4)c3)c3ccccc3-c3ccc(-n4c5ccccc5c5ccccc54)cc3)c(-c3ccc4ccccc4c3)c2)cc1 ; c1ccc(-c2ccc(N(c3cccc(-c4ccccc4)c3)c3ccccc3-c3ccc(-n4c5ccccc5c5ccccc54)cc3)cc2)cc1] 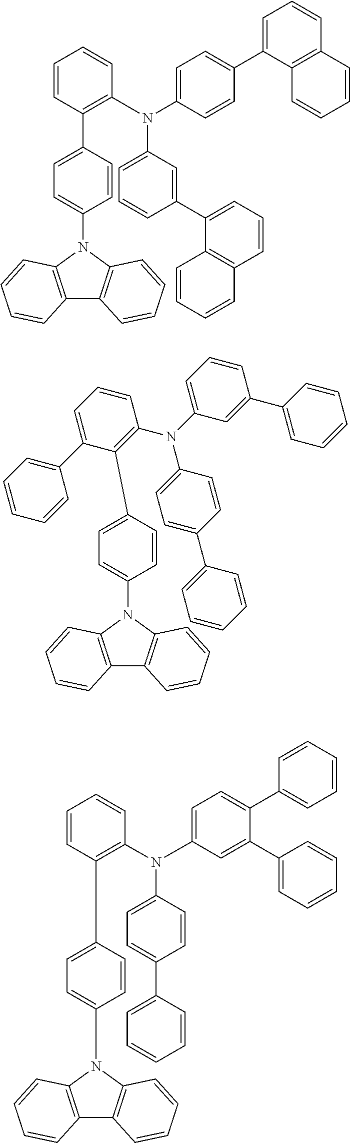 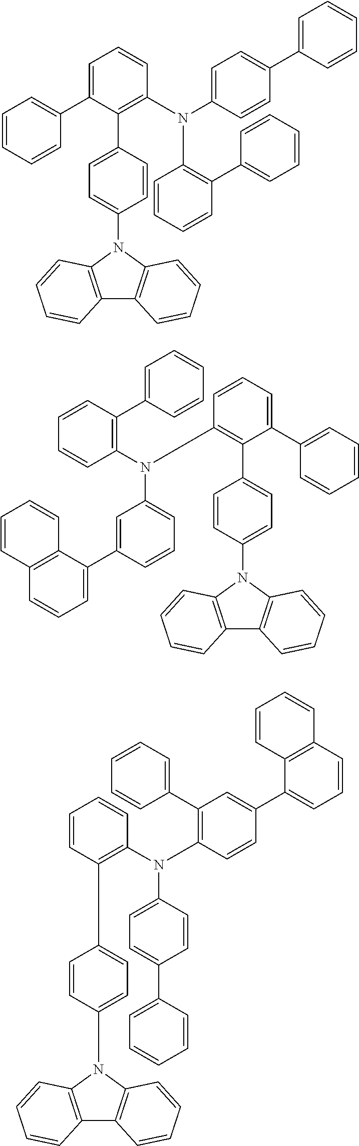 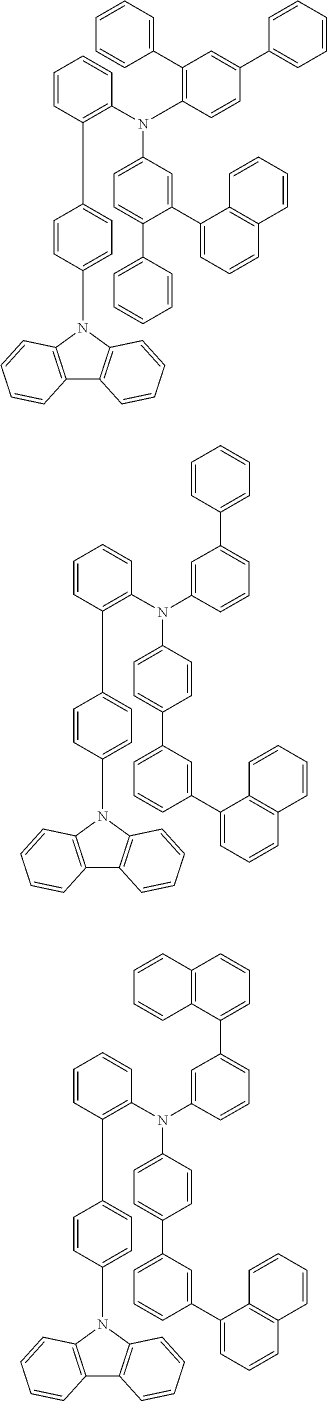 [SMILES: c1ccc(-c2ccc(N(c3ccc(-c4ccccc4)c(-c4cccc5ccccc45)c3)c3ccccc3-c3ccc(-n4c5ccccc5c5ccccc54)cc3)c(-c3ccccc3)c2)cc1 ; c1ccc(-c2cccc(N(c3ccc(-c4cccc(-c5cccc6ccccc56)c4)cc3)c3ccccc3-c3ccc(-n4c5ccccc5c5ccccc54)cc3)c2)cc1 ; c1cc(-c2ccc(N(c3cccc(-c4cccc5ccccc45)c3)c3ccccc3-c3ccc(-n4c5ccccc5c5ccccc54)cc3)cc2)cc(-c2cccc3ccccc23)c1] 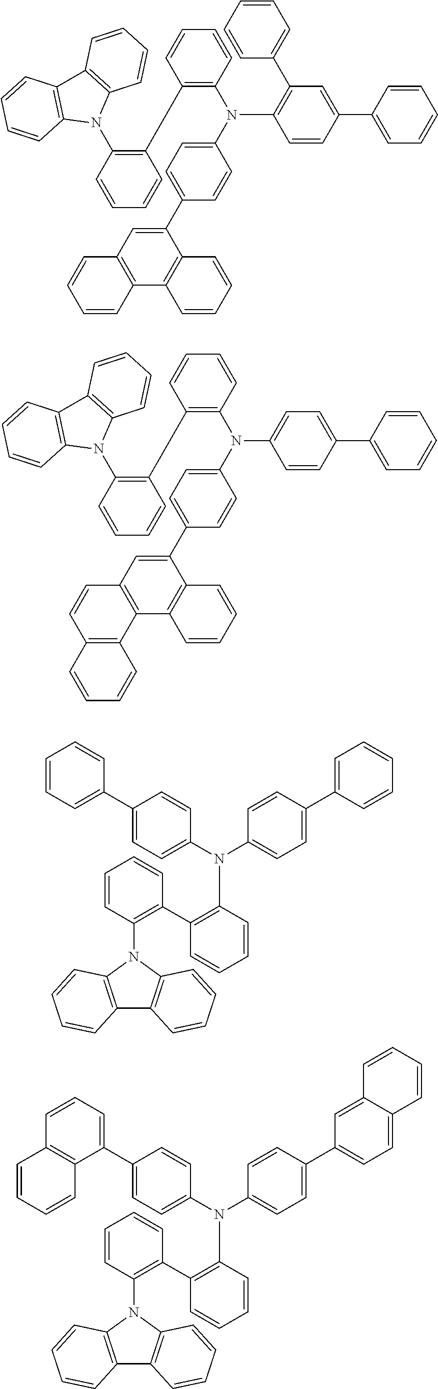 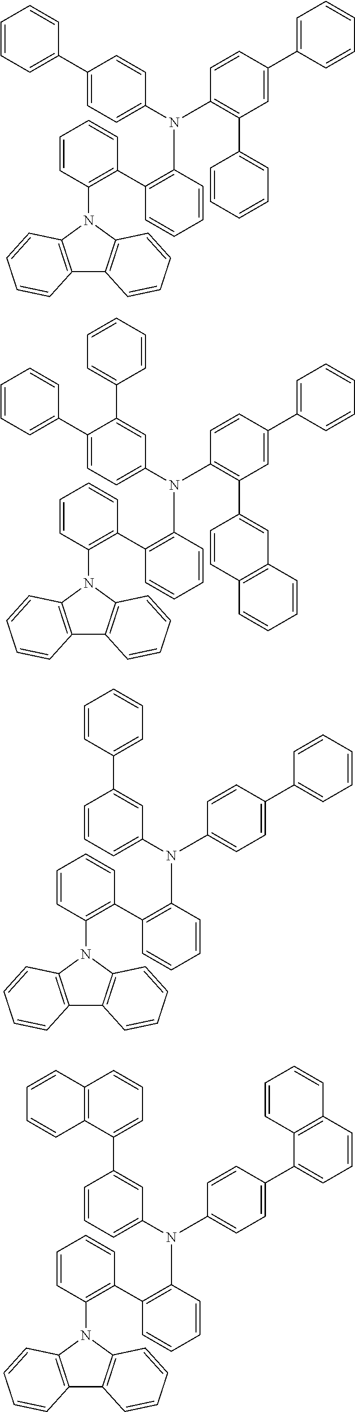 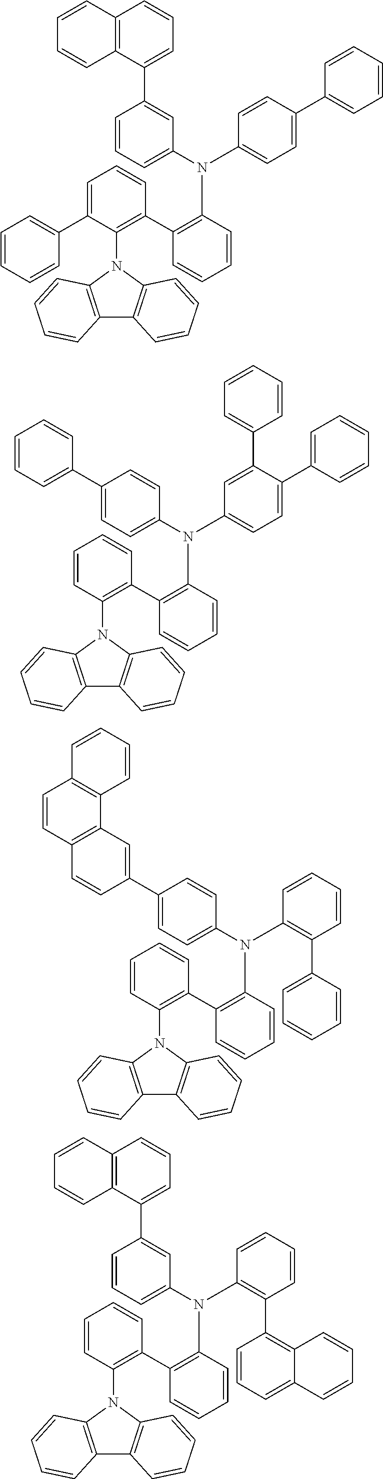 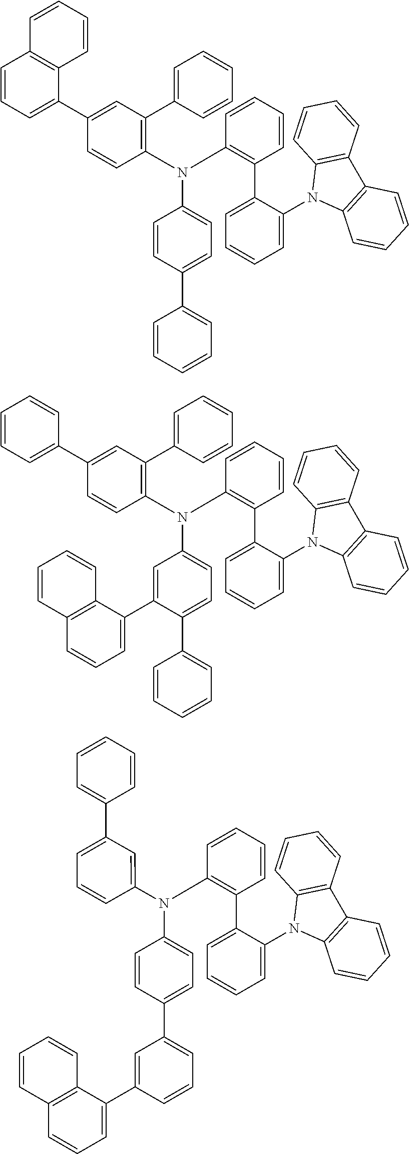 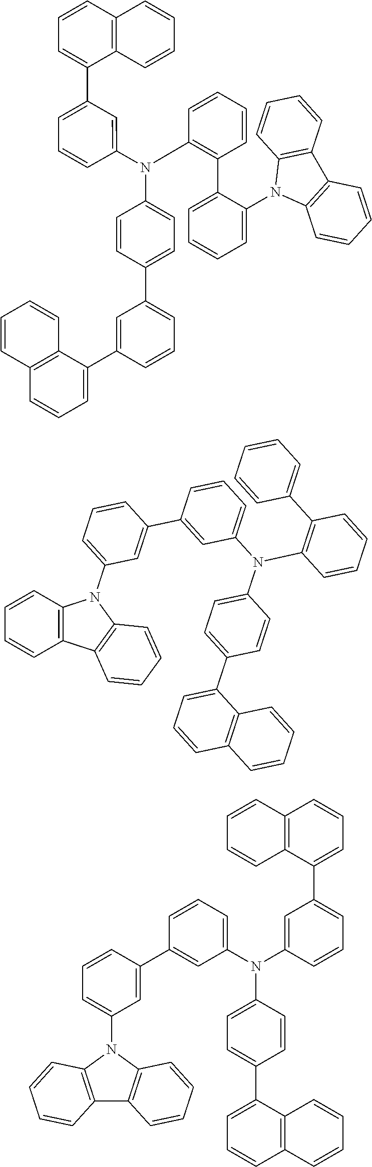 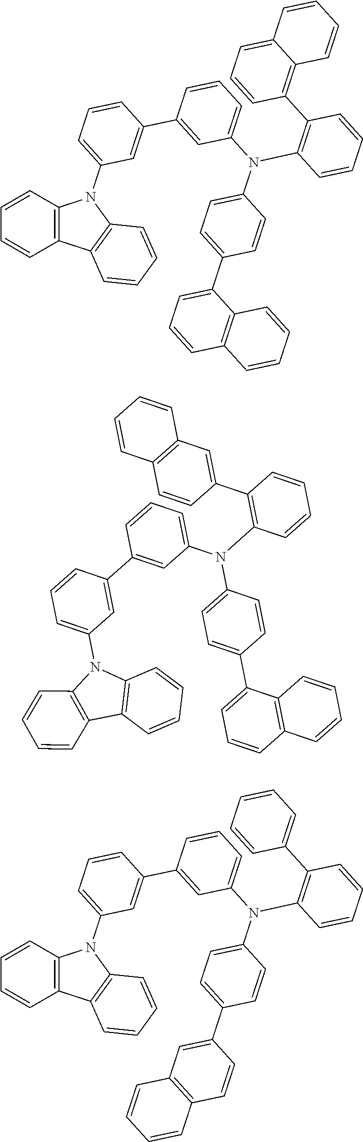 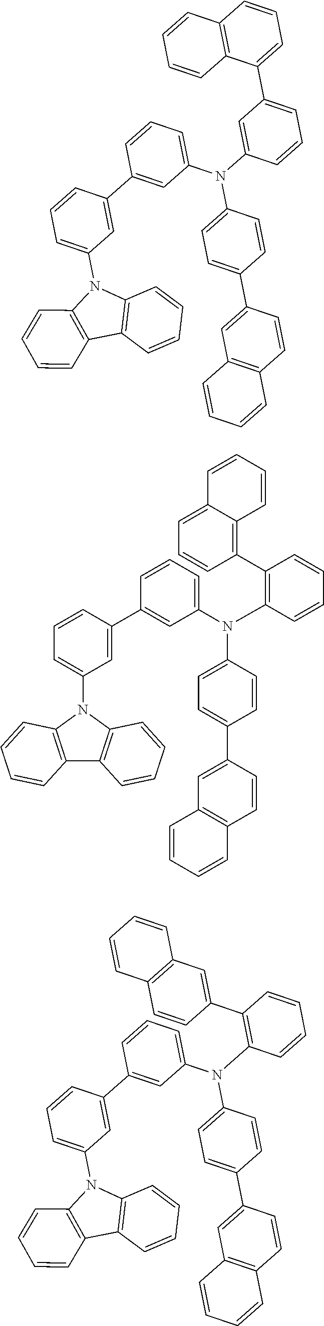 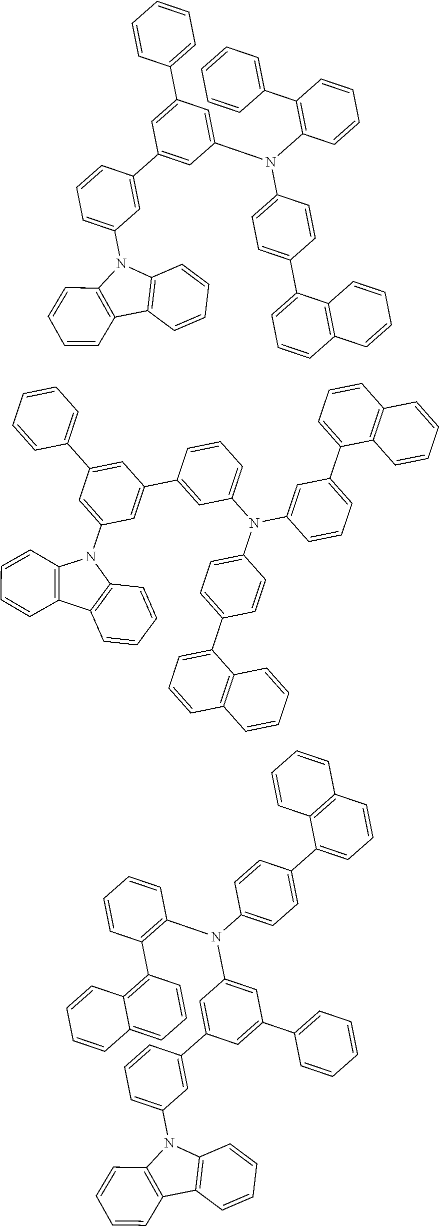 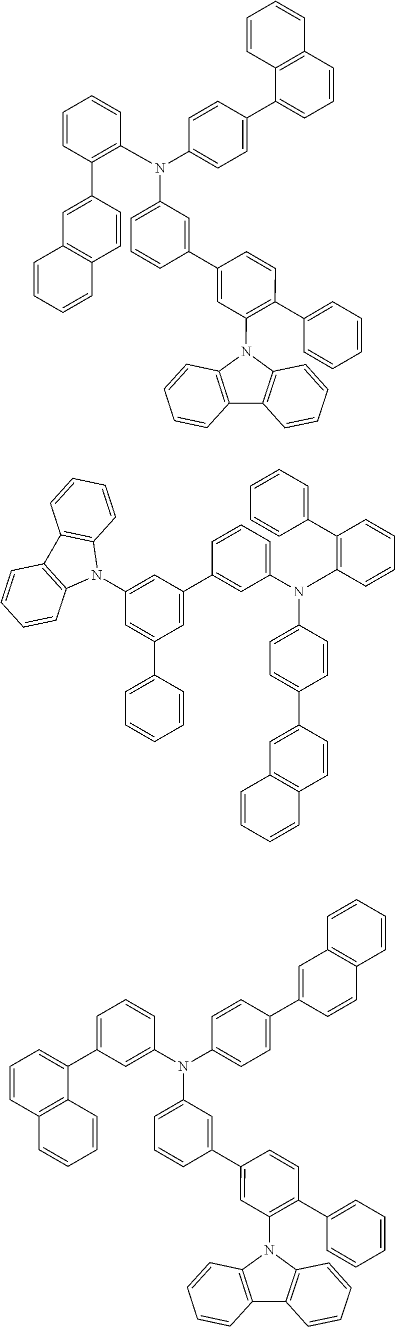 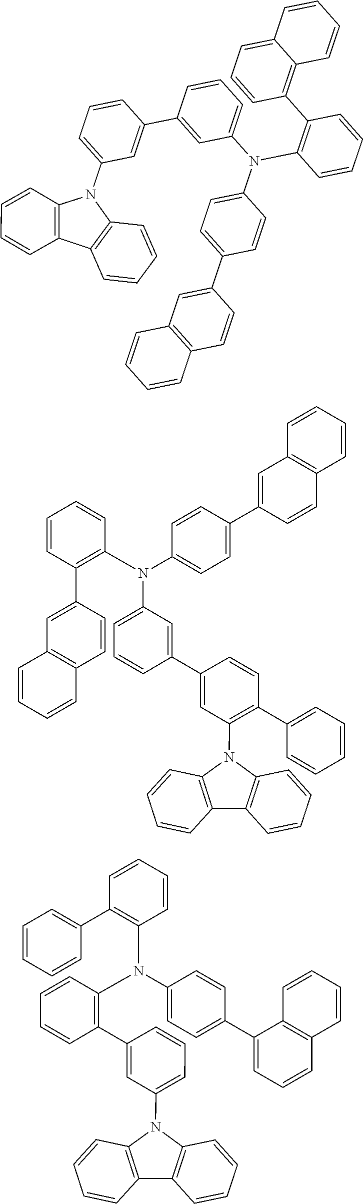 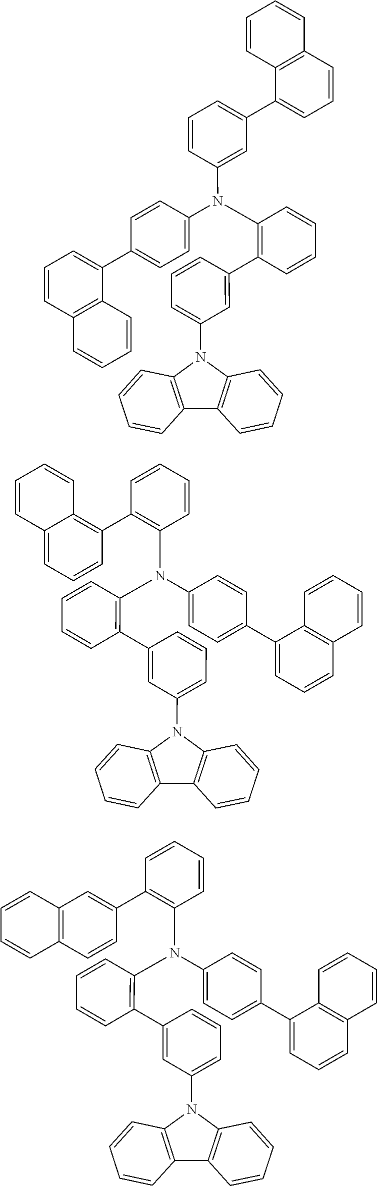 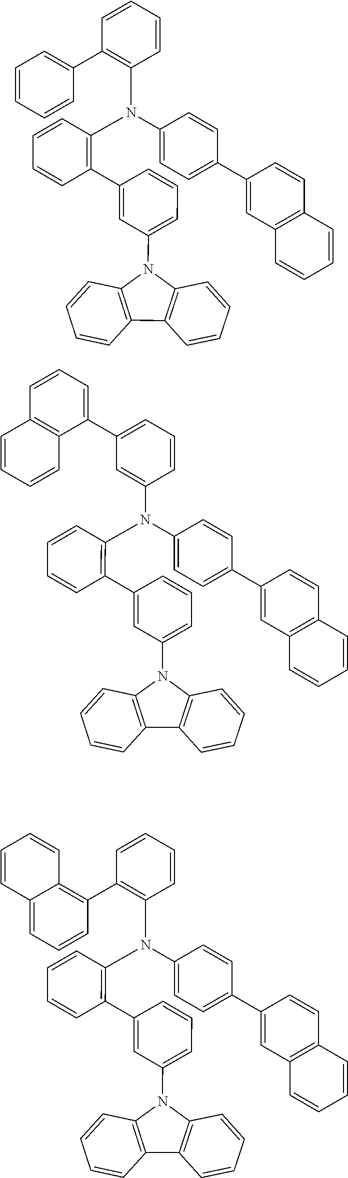 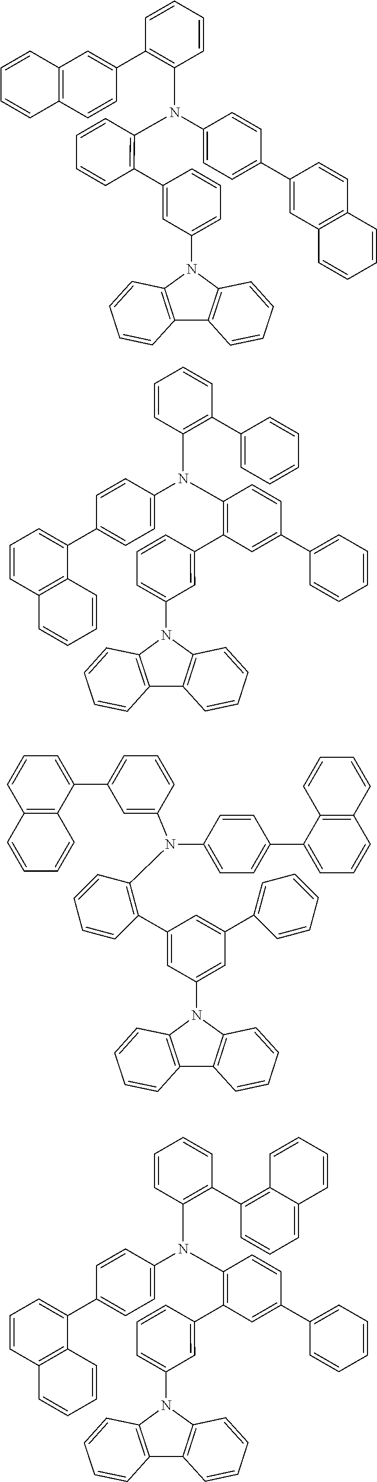 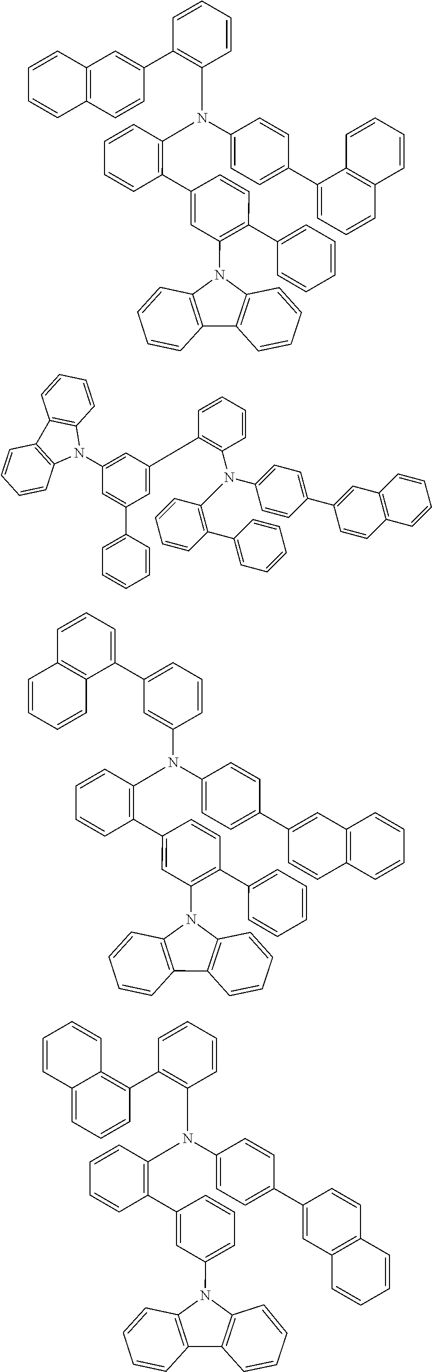 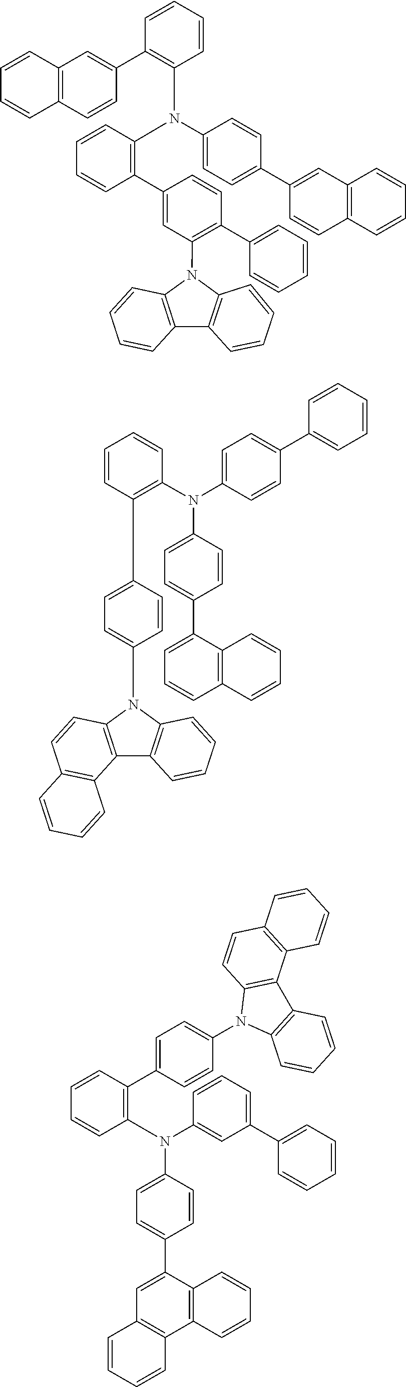 [SMILES: c1ccc(-c2ccc(-c3ccccc3N(c3ccc(-c4ccc5ccccc5c4)cc3)c3ccccc3-c3ccc4ccccc4c3)cc2-n2c3ccccc3c3ccccc32)cc1 ; c1ccc(-c2ccc(N(c3ccc(-c4cccc5ccccc45)cc3)c3ccccc3-c3ccc(-n4c5ccccc5c5c6ccccc6ccc54)cc3)cc2)cc1 ; c1ccc(-c2cccc(N(c3ccc(-c4cc5ccccc5c5ccccc45)cc3)c3ccccc3-c3ccc(-n4c5ccccc5c5c6ccccc6ccc54)cc3)c2)cc1] 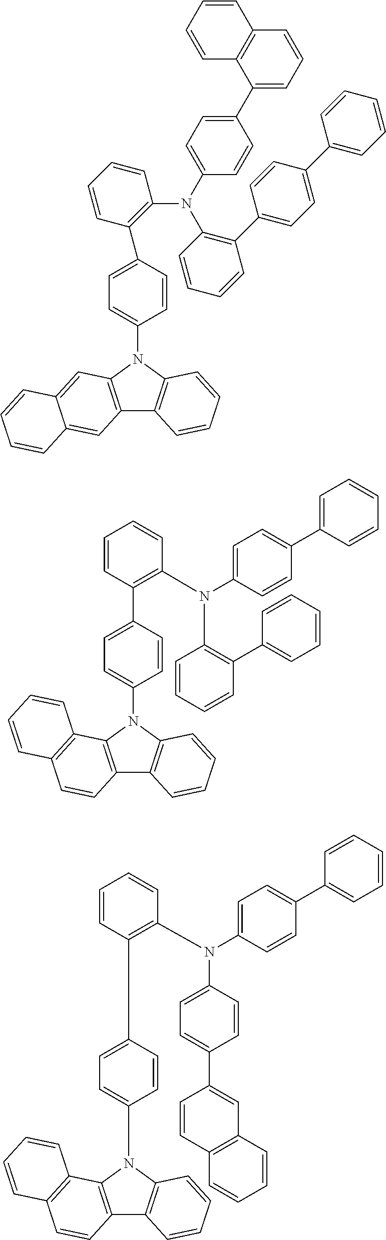 [SMILES: c1ccc(-c2ccc(-c3ccccc3N(c3ccc(-c4cccc5ccccc45)cc3)c3ccccc3-c3ccc(-n4c5ccccc5c5cc6ccccc6cc54)cc3)cc2)cc1 ; c1ccc(-c2ccc(N(c3ccccc3-c3ccccc3)c3ccccc3-c3ccc(-n4c5ccccc5c5ccc6ccccc6c54)cc3)cc2)cc1 ; c1ccc(-c2ccc(N(c3ccc(-c4ccc5ccccc5c4)cc3)c3ccccc3-c3ccc(-n4c5ccccc5c5ccc6ccccc6c54)cc3)cc2)cc1] 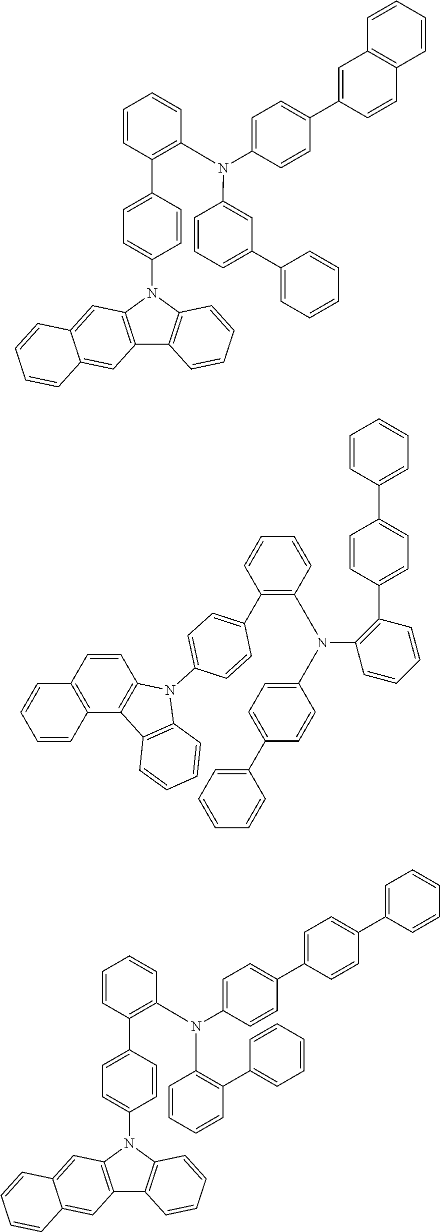 [SMILES: c1ccc(-c2cccc(N(c3ccc(-c4ccc5ccccc5c4)cc3)c3ccccc3-c3ccc(-n4c5ccccc5c5cc6ccccc6cc54)cc3)c2)cc1 ; c1ccc(-c2ccc(-c3ccccc3N(c3ccc(-c4ccccc4)cc3)c3ccccc3-c3ccc(-n4c5ccccc5c5c6ccccc6ccc54)cc3)cc2)cc1 ; c1ccc(-c2ccc(-c3ccc(N(c4ccccc4-c4ccccc4)c4ccccc4-c4ccc(-n5c6ccccc6c6cc7ccccc7cc65)cc4)cc3)cc2)cc1] 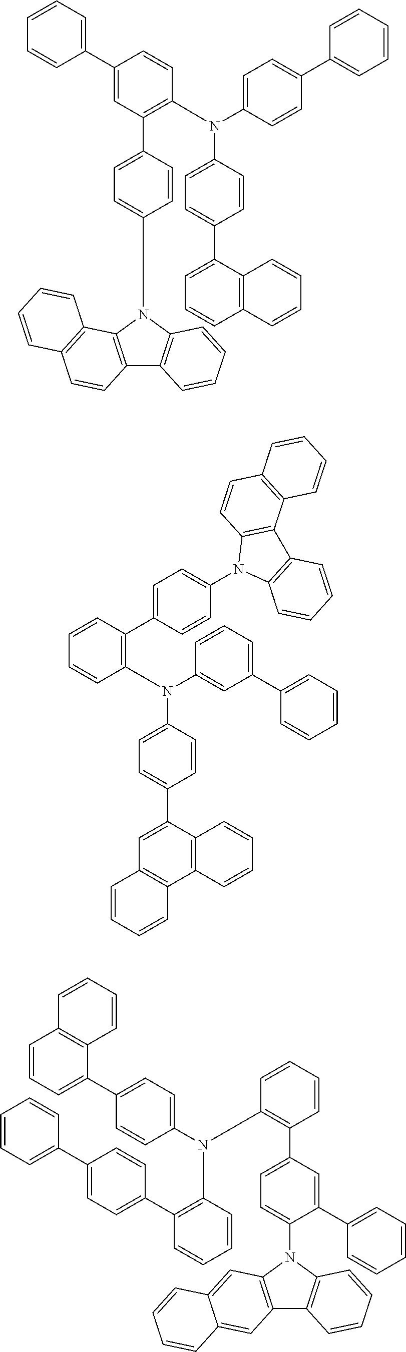 [SMILES: c1ccc(-c2ccc(N(c3ccc(-c4cccc5ccccc45)cc3)c3ccc(-c4ccccc4)cc3-c3ccc(-n4c5ccccc5c5ccc6ccccc6c54)cc3)cc2)cc1 ; c1ccc(-c2cccc(N(c3ccc(-c4cc5ccccc5c5ccccc45)cc3)c3ccccc3-c3ccc(-n4c5ccccc5c5c6ccccc6ccc54)cc3)c2)cc1 ; c1ccc(-c2ccc(-c3ccccc3N(Cc3ccccc3-c3ccc(-n4c5ccccc5c5cc6ccccc6cc54)c(-c4ccccc4)c3)c3ccc(-c4cccc5ccccc45)cc3)cc2)cc1] 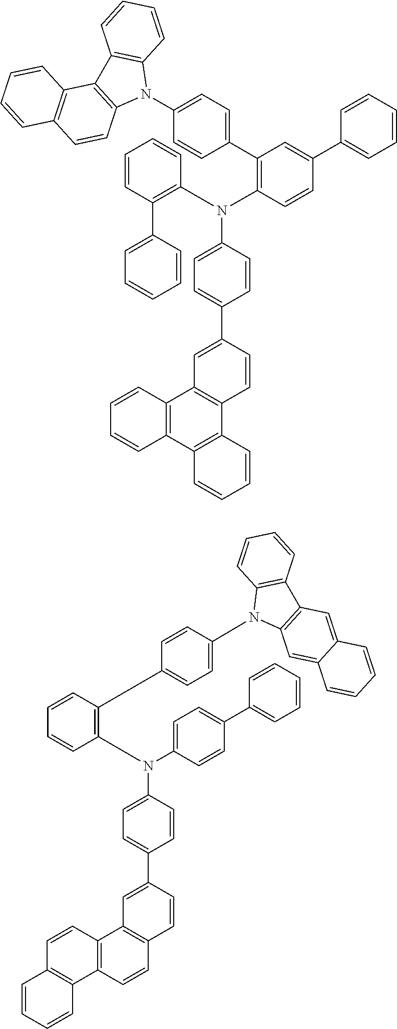 [SMILES: c1ccc(-c2ccc(N(c3ccc(-c4ccc5c6ccccc6c6ccccc6c5c4)cc3)c3ccccc3-c3ccccc3)c(-c3ccc(-n4c5ccccc5c5c6ccccc6ccc54)cc3)c2)cc1 ; c1ccc(-c2ccc(N(c3ccc(-c4ccc5ccc6c7ccccc7ccc6c5c4)cc3)c3ccccc3-c3ccc(-n4c5ccccc5c5cc6ccccc6cc54)cc3)cc2)cc1] 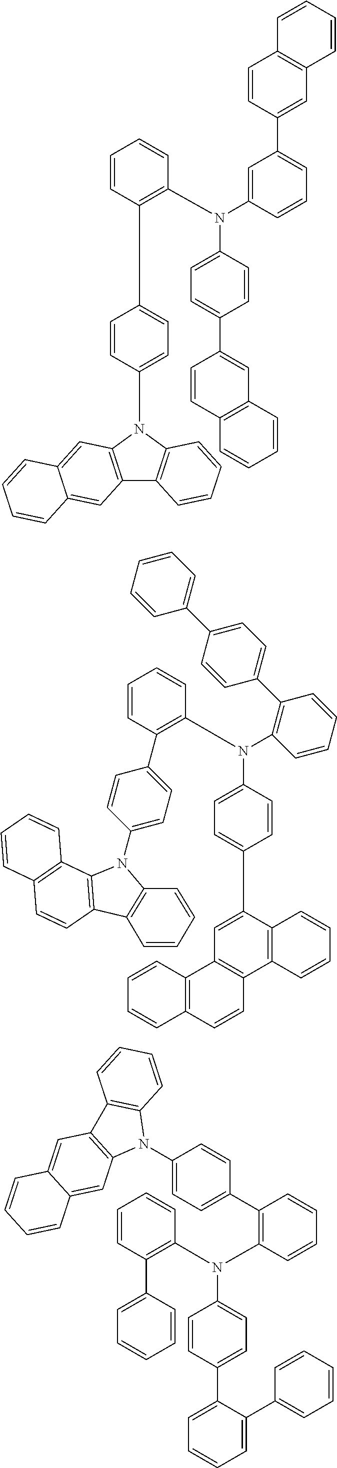 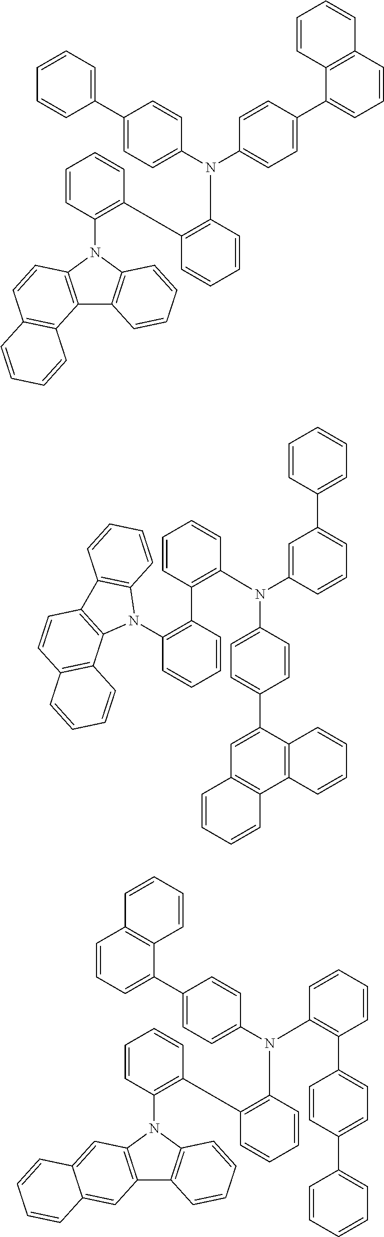 [SMILES: c1ccc(-c2ccc(N(c3ccc(-c4cccc5ccccc45)cc3)c3ccccc3Cc3ccccc3-n3c4ccccc4c4c5ccccc5ccc43)cc2)cc1 ; c1ccc(-c2cccc(N(c3ccc(-c4cc5ccccc5c5ccccc45)cc3)c3ccccc3-c3ccccc3-n3c4ccccc4c4ccc5ccccc5c43)c2)cc1 ; c1ccc(-c2ccc(-c3ccccc3N(c3ccc(-c4cccc5ccccc45)cc3)c3ccccc3Cc3ccccc3-n3c4ccccc4c4cc5ccccc5cc43)cc2)cc1] 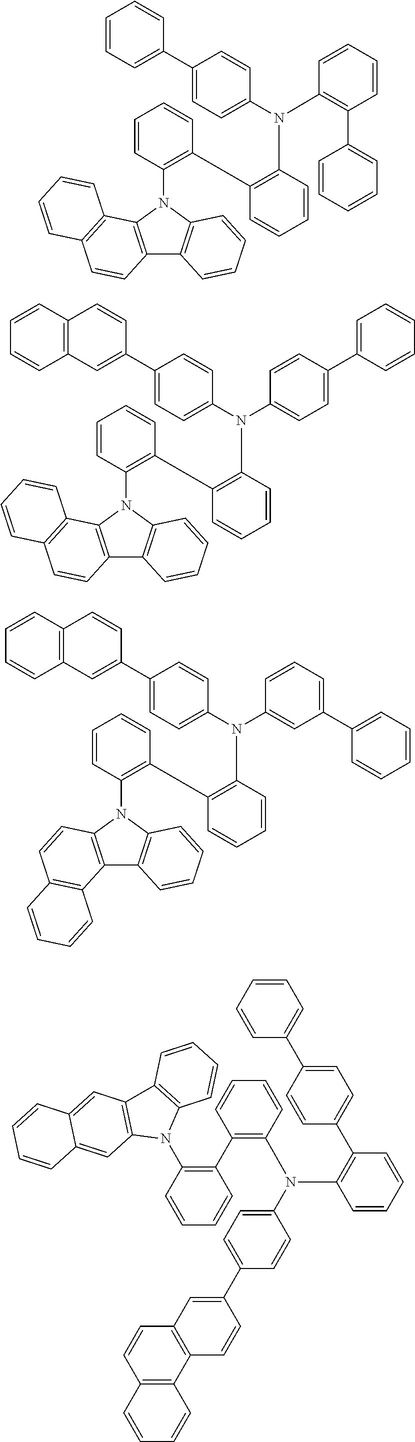 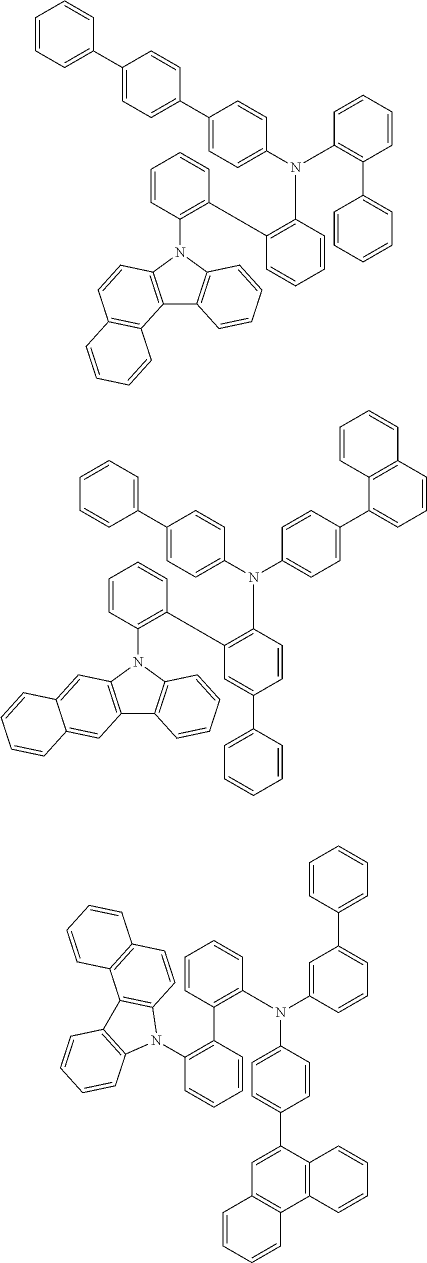 [SMILES: c1ccc(-c2ccc(-c3ccc(N(c4ccccc4Cc4ccccc4-n4c5ccccc5c5c6ccccc6ccc54)c4ccccc4-c4ccccc4)cc3)cc2)cc1 ; c1ccc(-c2ccc(N(c3ccc(-c4cccc5ccccc45)cc3)c3ccc(-c4ccccc4)cc3Cc3ccccc3-n3c4ccccc4c4cc5ccccc5cc43)cc2)cc1 ; c1ccc(-c2cccc(N(c3ccc(-c4cc5ccccc5c5ccccc45)cc3)c3ccccc3-c3ccccc3-n3c4ccccc4c4c5ccccc5ccc43)c2)cc1] 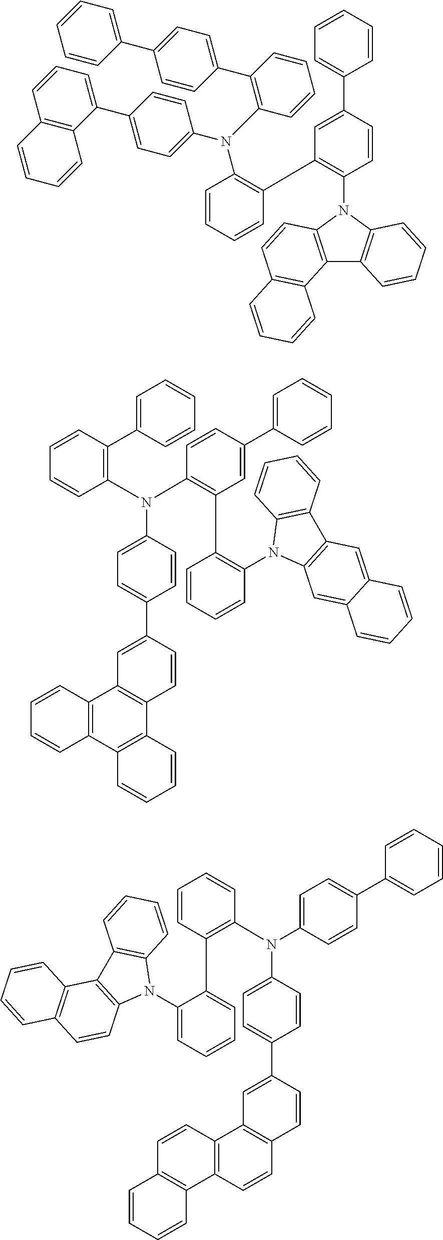 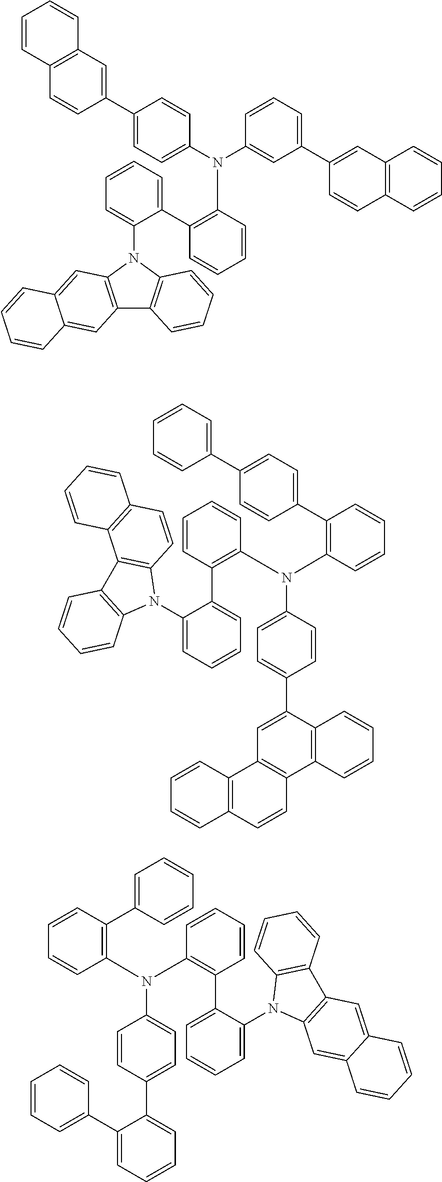 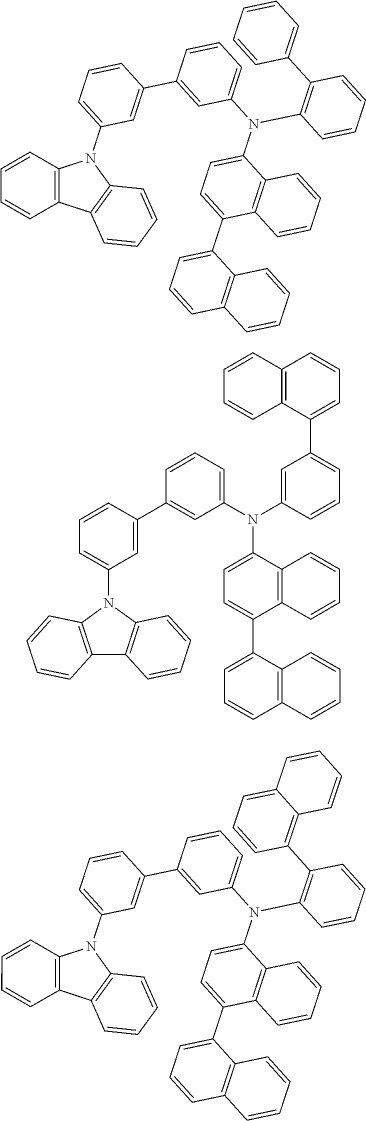 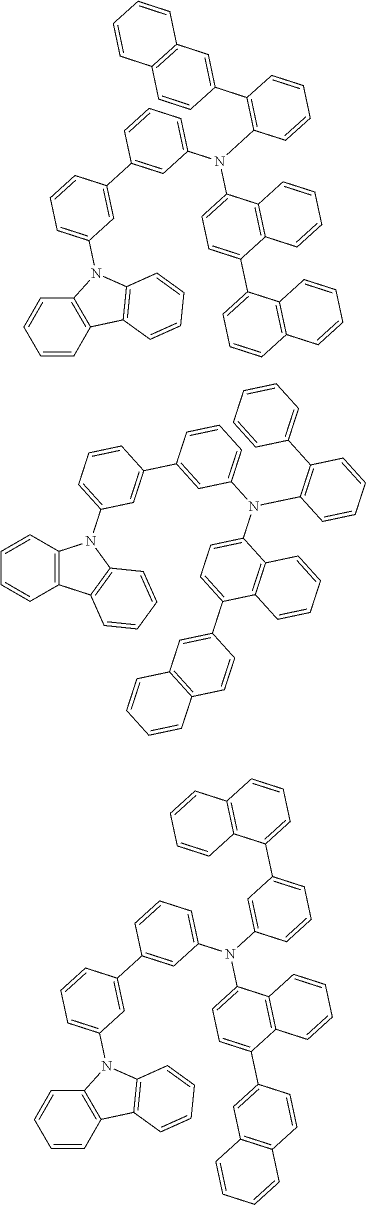 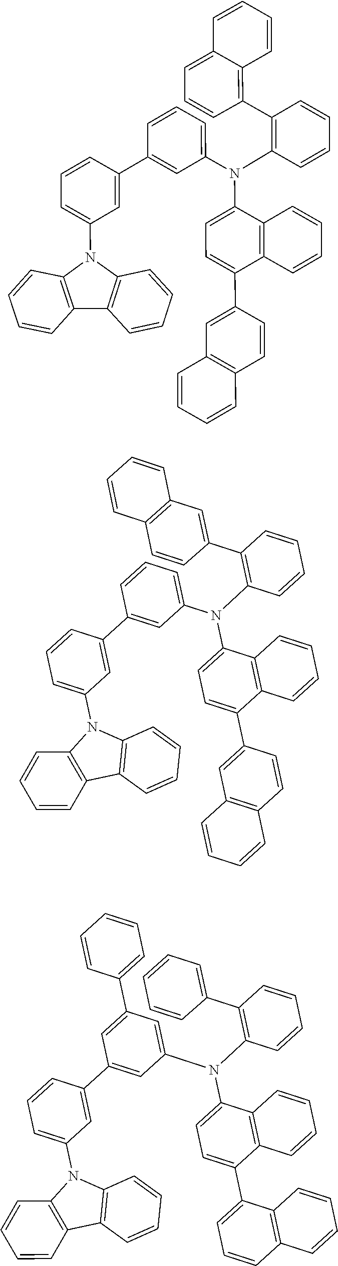 [SMILES: c1cc(-c2cccc(-n3c4ccccc4c4ccccc43)c2)cc(N(c2ccccc2-c2cccc3ccccc23)c2ccc(-c3ccc4ccccc4c3)c3ccccc23)c1 ; c1cc(-c2cccc(-n3c4ccccc4c4ccccc43)c2)cc(N(c2ccccc2-c2ccc3ccccc3c2)c2ccc(-c3ccc4ccccc4c3)c3ccccc23)c1 ; c1ccc(-c2cc(-c3cccc(-n4c5ccccc5c5ccccc54)c3)cc(N(c3ccccc3-c3ccccc3)c3ccc(-c4cccc5ccccc45)c4ccccc34)c2)cc1] 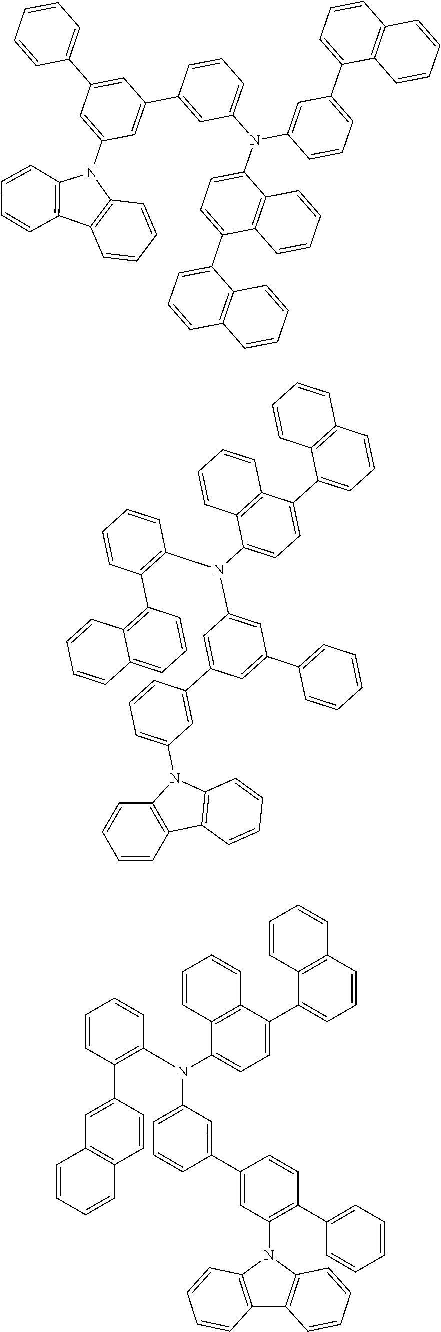 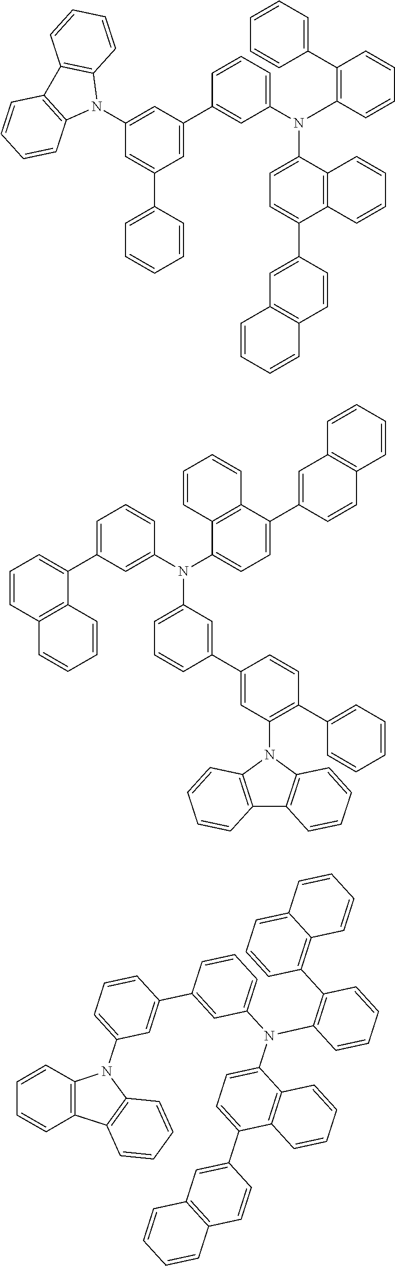 [SMILES: c1ccc(-c2cc(-c3cccc(N(c4ccccc4-c4ccccc4)c4ccc(-c5ccc6ccccc6c5)c5ccccc45)c3)cc(-n3c4ccccc4c4ccccc43)c2)cc1 ; c1ccc(-c2ccc(-c3cccc(N(c4cccc(-c5cccc6ccccc56)c4)c4ccc(-c5ccc6ccccc6c5)c5ccccc45)c3)cc2-n2c3ccccc3c3ccccc32)cc1 ; c1cc(-c2cccc(-n3c4ccccc4c4ccccc43)c2)cc(N(c2ccccc2-c2cccc3ccccc23)c2ccc(-c3ccc4ccccc4c3)c3ccccc23)c1] 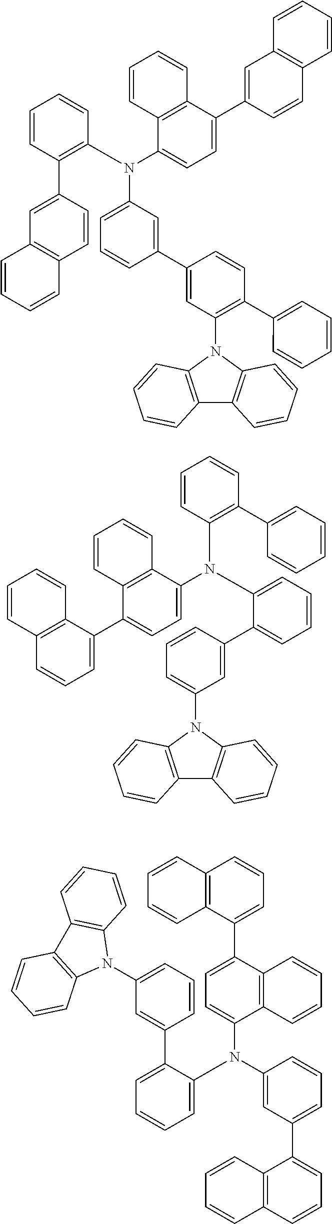 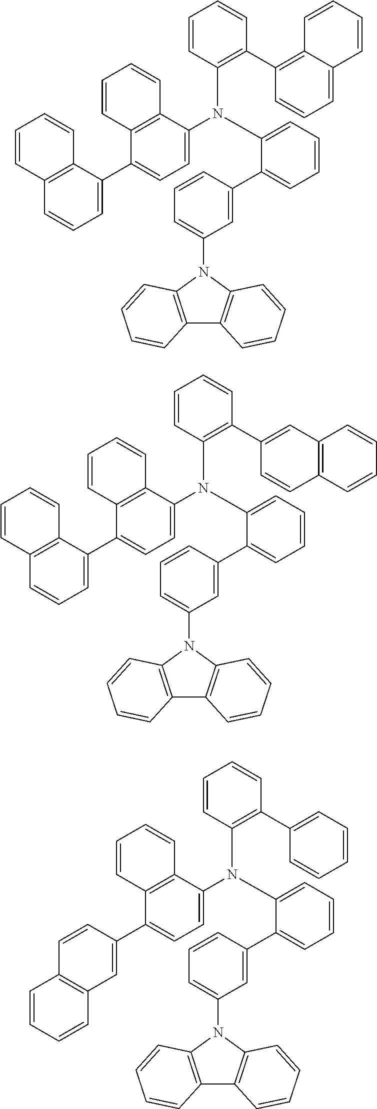 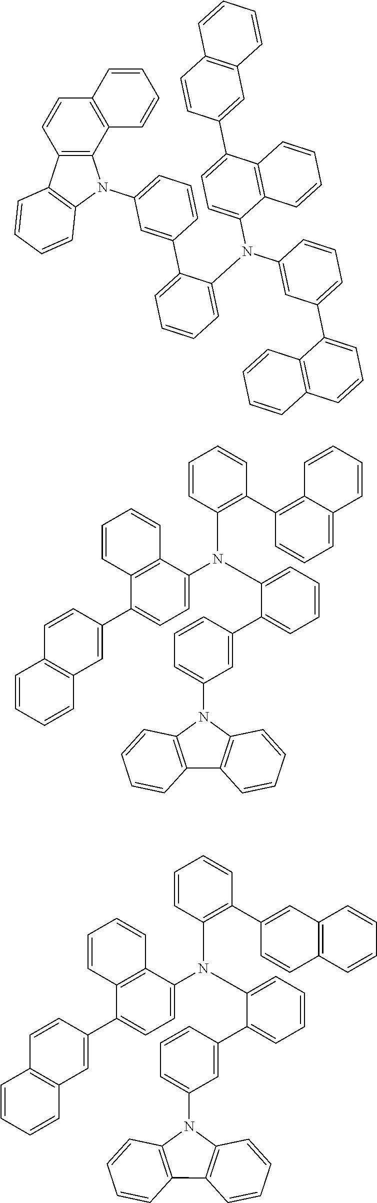 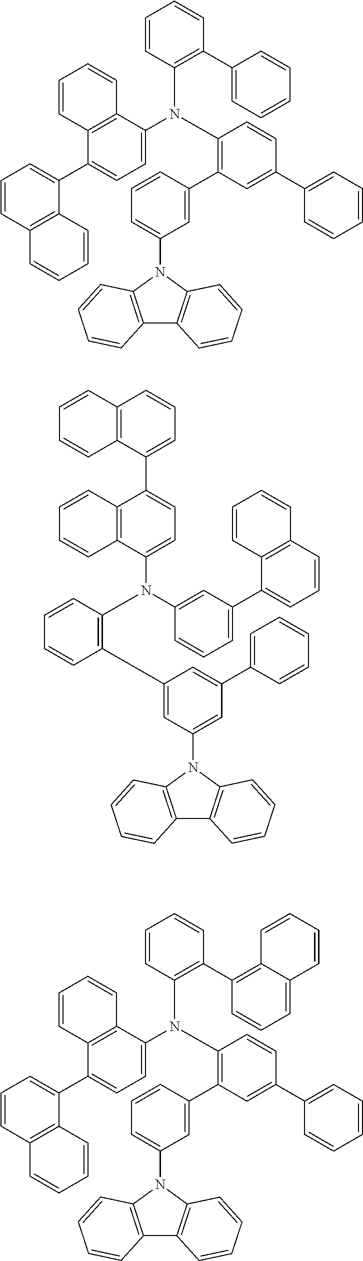 [SMILES: c1ccc(-c2ccc(N(c3ccccc3-c3ccccc3)c3ccc(-c4cccc5ccccc45)c4ccccc34)c(-c3cccc(-n4c5ccccc5c5ccccc54)c3)c2)cc1 ; c1ccc(-c2cc(-c3ccccc3N(c3cccc(-c4cccc5ccccc45)c3)c3ccc(-c4cccc5ccccc45)c4ccccc34)cc(-n3c4ccccc4c4ccccc43)c2)cc1 ; c1ccc(-c2ccc(N(c3ccccc3-c3cccc4ccccc34)c3ccc(-c4cccc5ccccc45)c4ccccc34)c(-c3cccc(-n4c5ccccc5c5ccccc54)c3)c2)cc1] 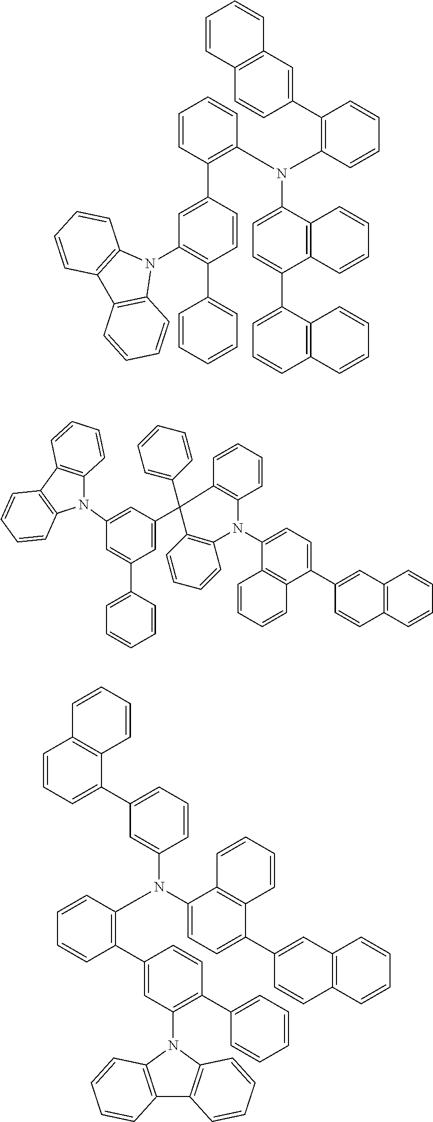 [SMILES: c1ccc(-c2ccc(-c3ccccc3N(c3ccccc3-c3ccc4ccccc4c3)c3ccc(-c4cccc5ccccc45)c4ccccc34)cc2-n2c3ccccc3c3ccccc32)cc1 ; c1ccc(-c2cc(-n3c4ccccc4c4ccccc43)cc(C3(c4ccccc4)c4ccccc4N(c4ccc(-c5ccc6ccccc6c5)c5ccccc45)c4ccccc43)c2)cc1 ; c1ccc(-c2ccc(-c3ccccc3N(c3cccc(-c4cccc5ccccc45)c3)c3ccc(-c4ccc5ccccc5c4)c4ccccc34)cc2-n2c3ccccc3c3ccccc32)cc1] 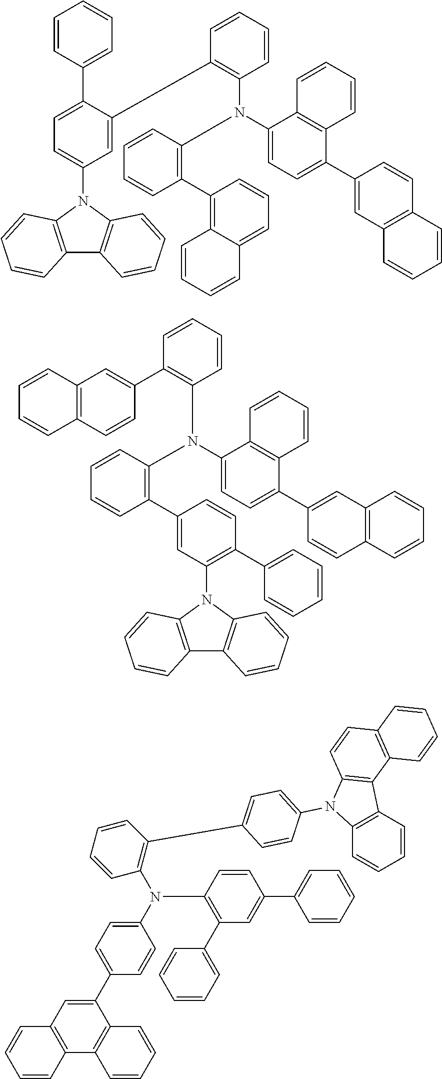 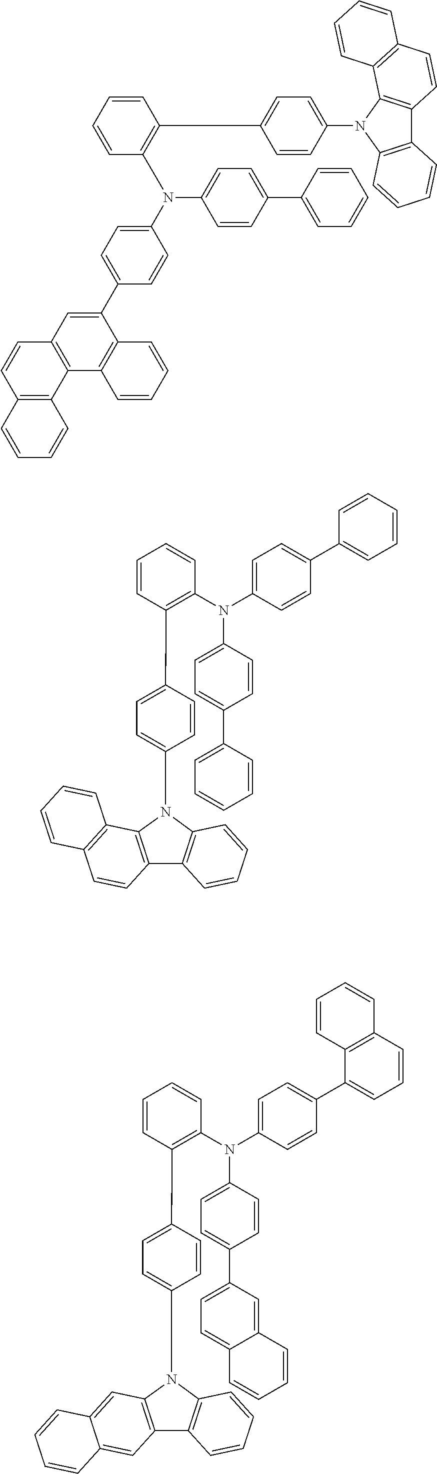 [SMILES: c1ccc(-c2ccc(N(c3ccc(-c4cc5ccc6ccccc6c5c5ccccc45)cc3)c3ccccc3-c3ccc(-n4c5ccccc5c5ccc6ccccc6c54)cc3)cc2)cc1 ; c1ccc(-c2ccc(N(c3ccc(-c4ccccc4)cc3)c3ccccc3-c3ccc(-n4c5ccccc5c5ccc6ccccc6c54)cc3)cc2)cc1 ; c1ccc(N(c2ccc(-c3ccc4ccccc4c3)cc2)c2ccc(-c3cccc4ccccc34)cc2)c(-c2ccc(-n3c4ccccc4c4cc5ccccc5cc43)cc2)c1] 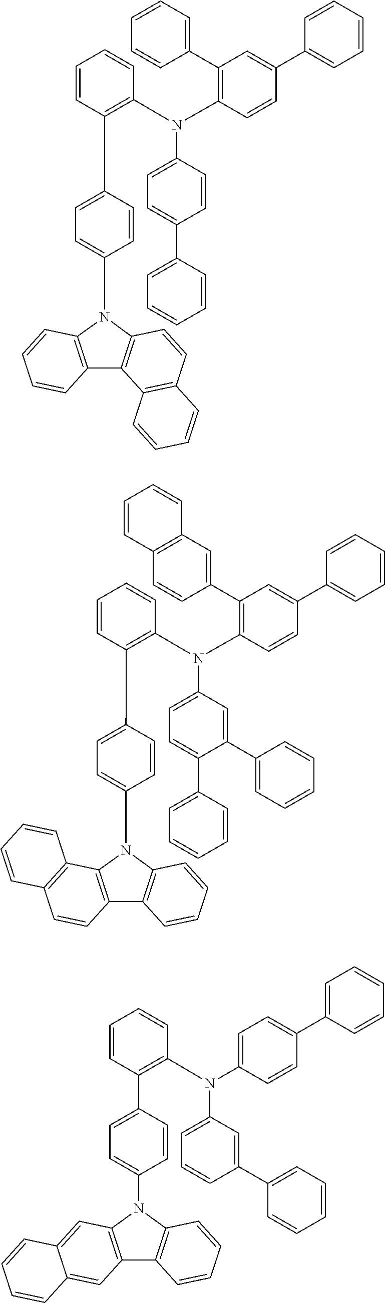 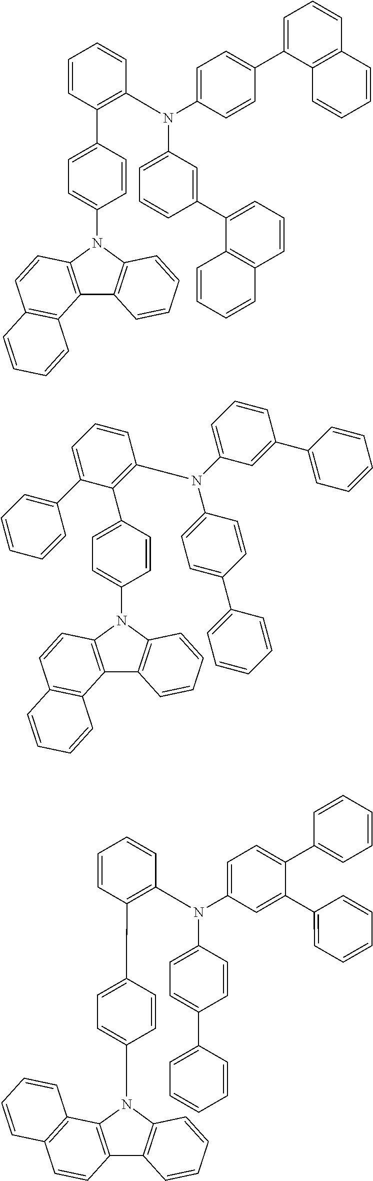 [SMILES: c1cc(-c2cccc3ccccc23)cc(N(c2ccc(-c3cccc4ccccc34)cc2)c2ccccc2-c2ccc(-n3c4ccccc4c4c5ccccc5ccc43)cc2)c1 ; c1ccc(-c2ccc(N(c3cccc(-c4ccccc4)c3)c3cccc(-c4ccccc4)c3-c3ccc(-n4c5ccccc5c5c6ccccc6ccc54)cc3)cc2)cc1 ; c1ccc(-c2ccc(N(c3ccc(-c4ccccc4)c(-c4ccccc4)c3)c3ccccc3-c3ccc(-n4c5ccccc5c5ccc6ccccc6c54)cc3)cc2)cc1] 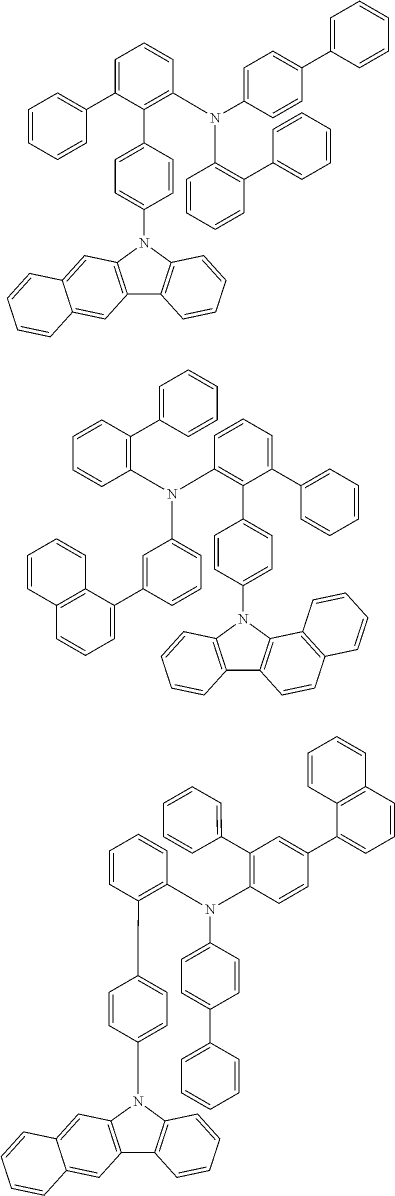 [SMILES: c1ccc(-c2ccc(N(c3ccccc3-c3ccccc3)c3cccc(-c4ccccc4)c3-c3ccc(-n4c5ccccc5c5cc6ccccc6cc54)cc3)cc2)cc1 ; c1ccc(-c2ccccc2N(c2cccc(-c3cccc4ccccc34)c2)c2cccc(-c3ccccc3)c2-c2ccc(-n3c4ccccc4c4ccc5ccccc5c43)cc2)cc1 ; c1ccc(-c2ccc(N(c3ccccc3-c3ccc(-n4c5ccccc5c5cc6ccccc6cc54)cc3)c3ccc(-c4cccc5ccccc45)cc3-c3ccccc3)cc2)cc1] 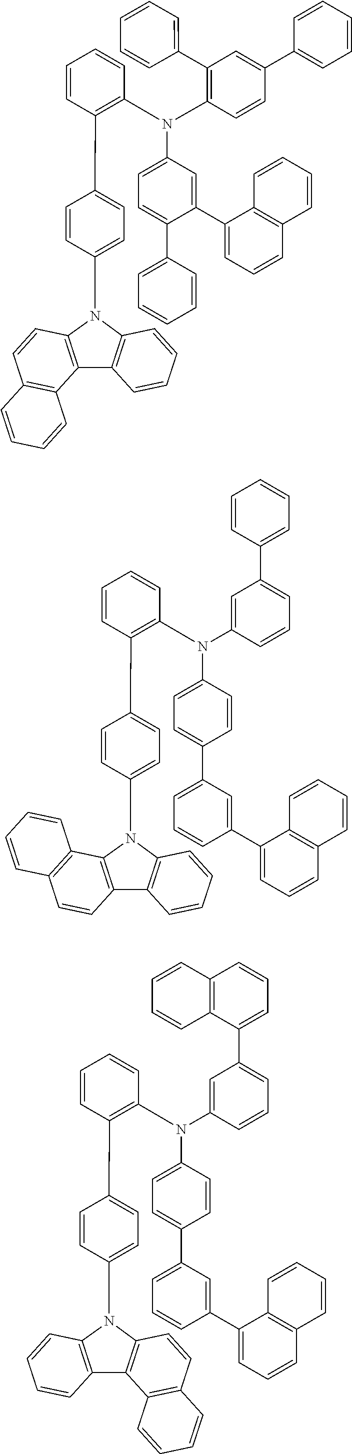 [SMILES: c1ccc(-c2ccc(N(c3ccc(-c4ccccc4)c(-c4cccc5ccccc45)c3)c3ccccc3-c3ccc(-n4c5ccccc5c5c6ccccc6ccc54)cc3)c(-c3ccccc3)c2)cc1 ; c1ccc(-c2cccc(N(c3ccc(-c4cccc(-c5cccc6ccccc56)c4)cc3)c3ccccc3-c3ccc(-n4c5ccccc5c5ccc6ccccc6c54)cc3)c2)cc1 ; c1cc(-c2ccc(N(c3cccc(-c4cccc5ccccc45)c3)c3ccccc3-c3ccc(-n4c5ccccc5c5c6ccccc6ccc54)cc3)cc2)cc(-c2cccc3ccccc23)c1] 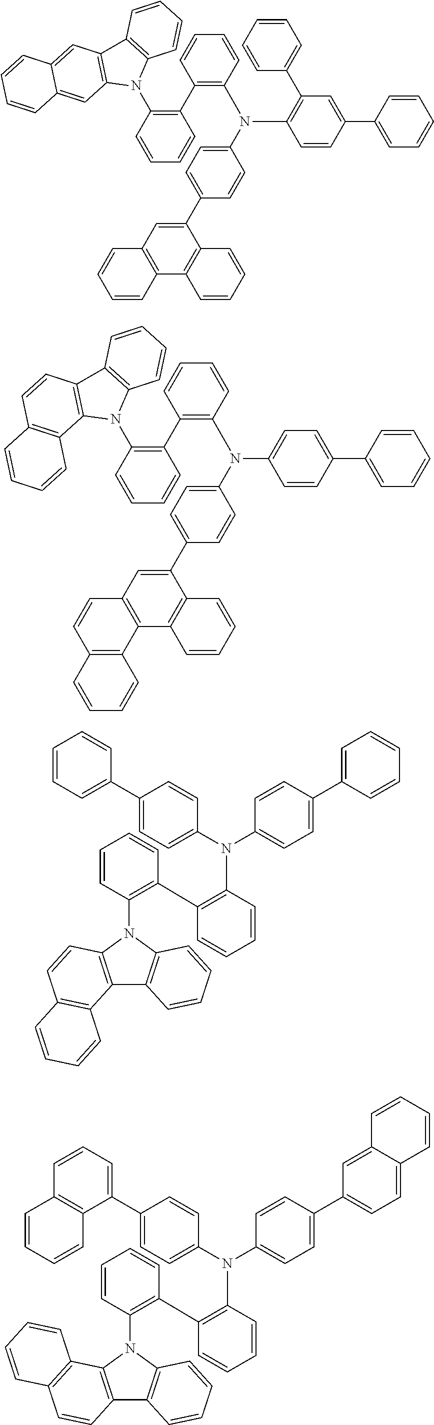 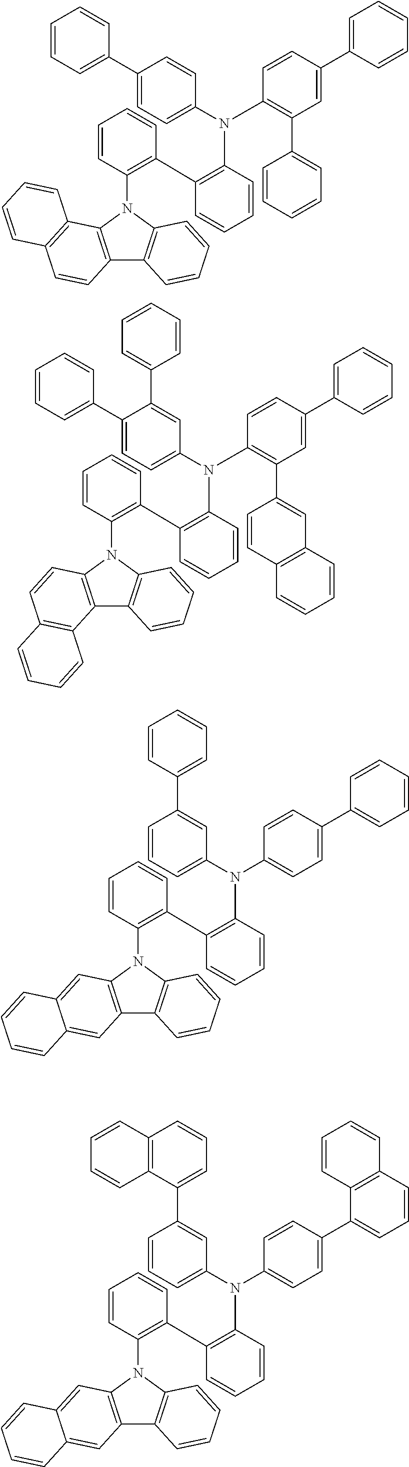 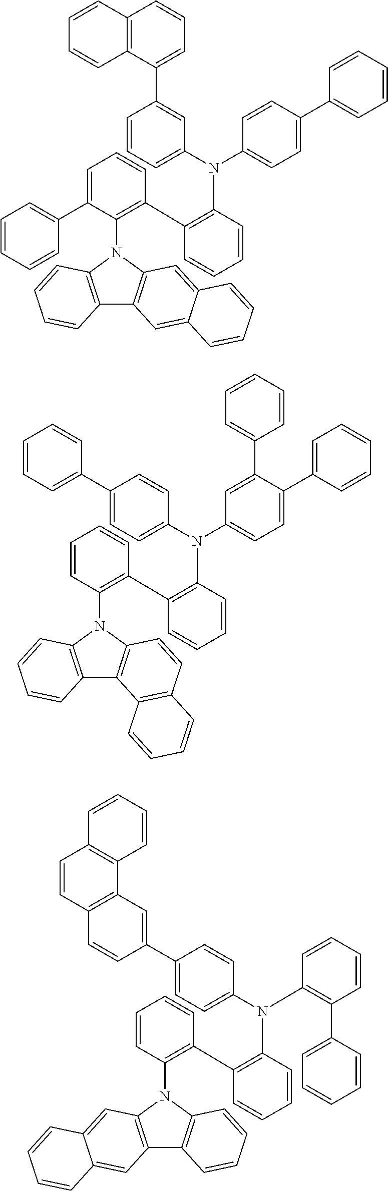 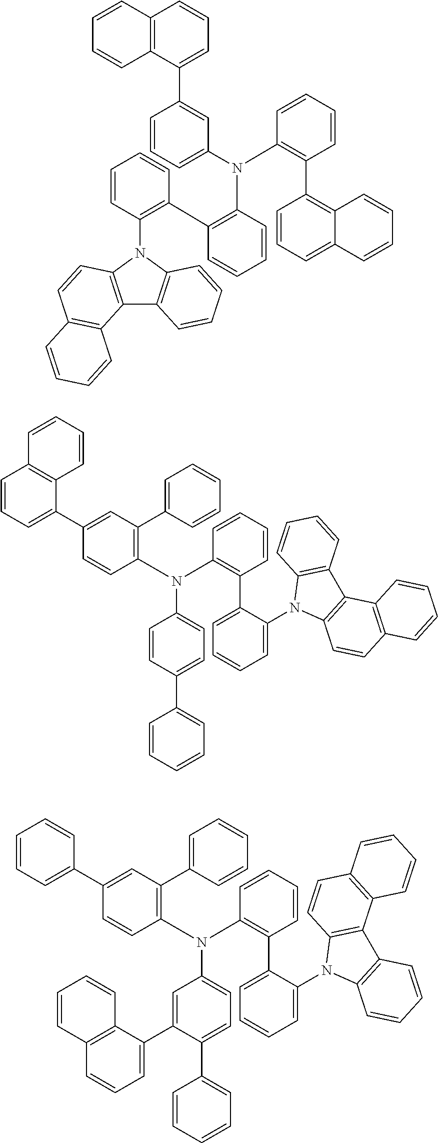 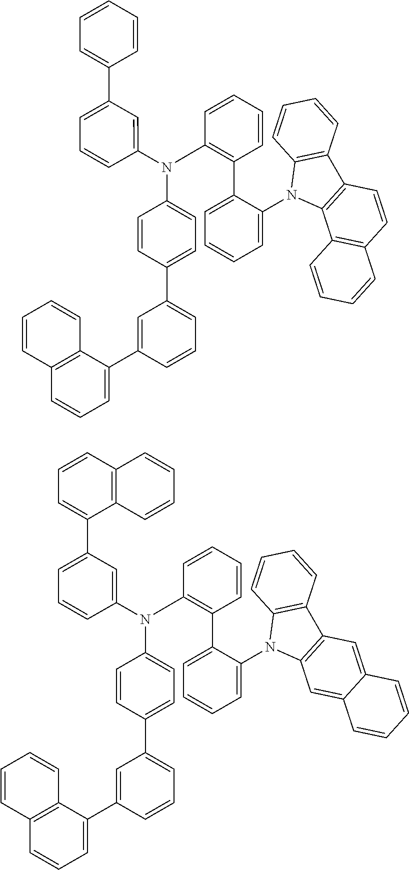 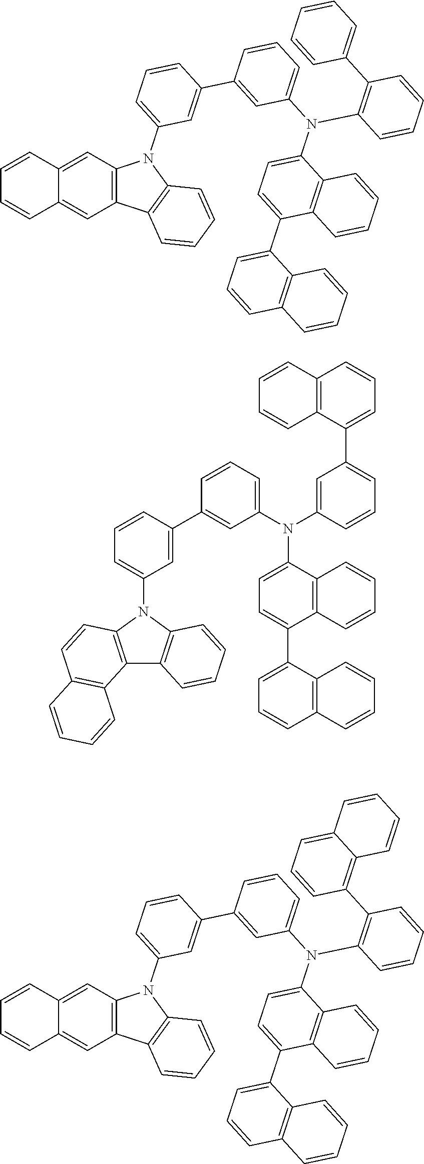 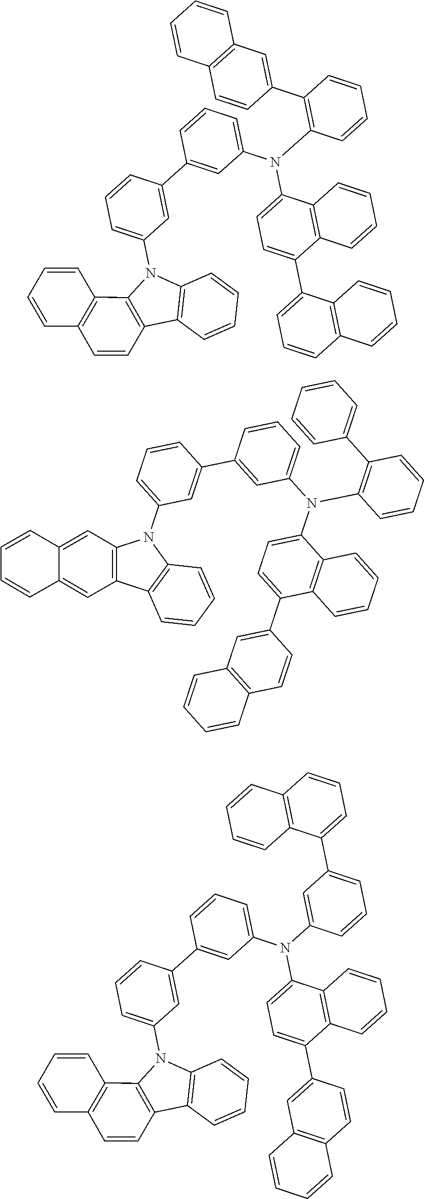 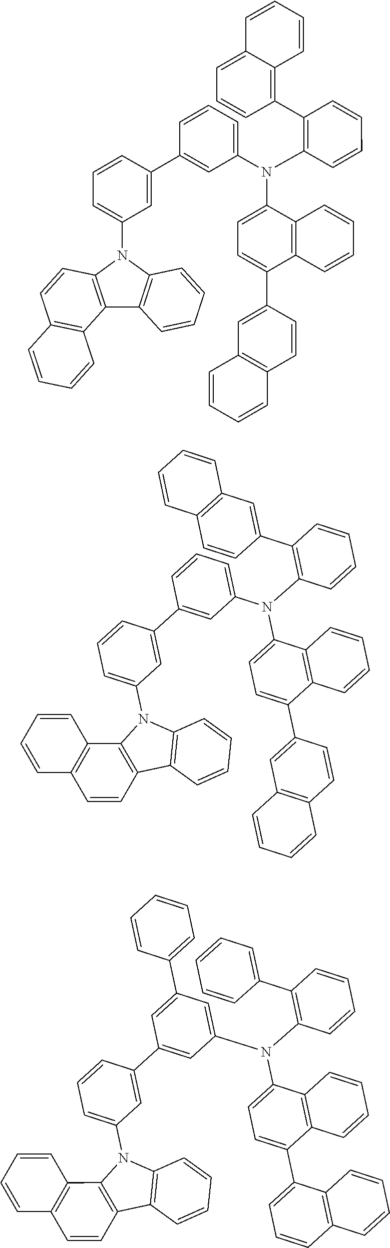 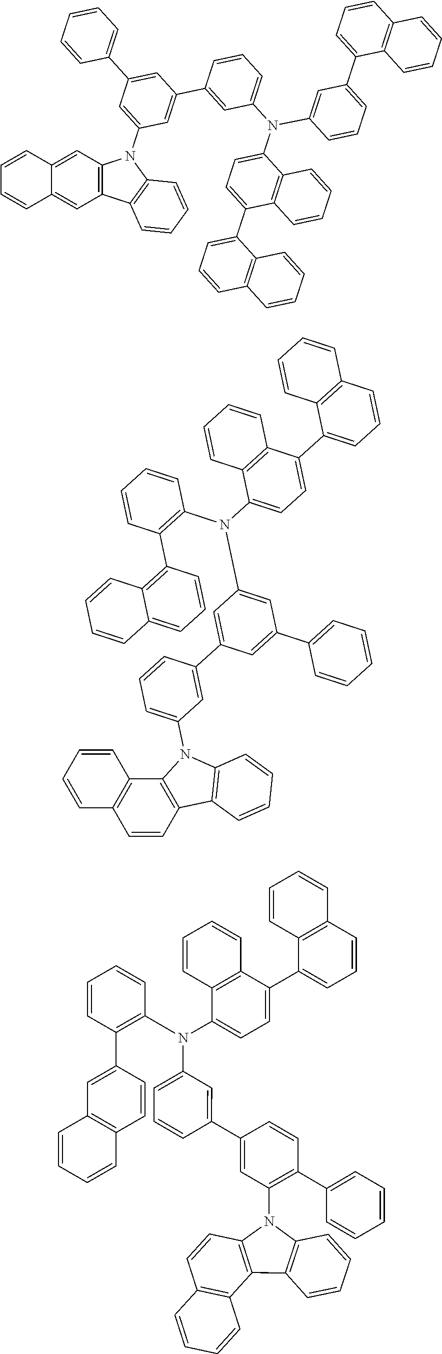 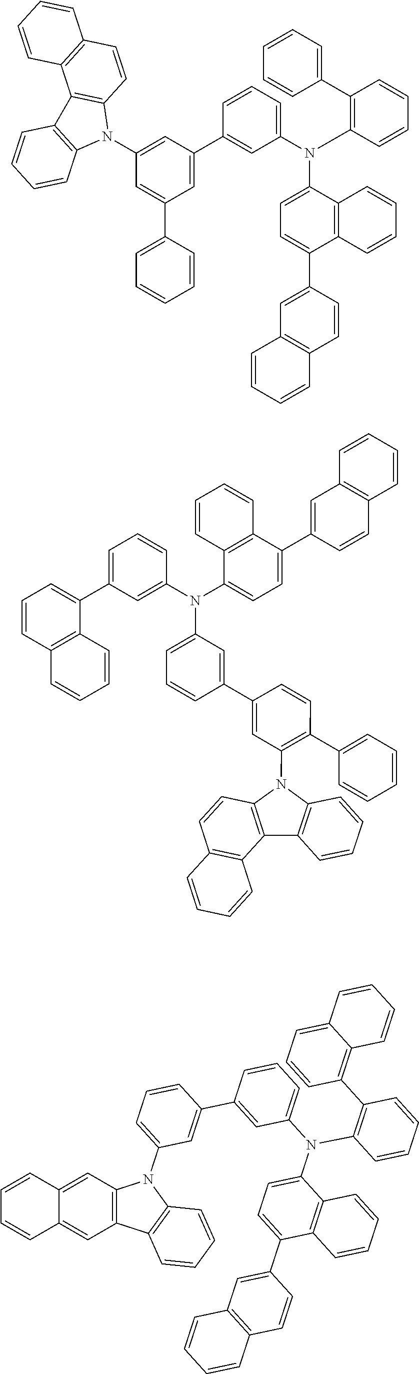 [SMILES: c1ccc(-c2cc(-c3cccc(N(c4ccccc4-c4ccccc4)c4ccc(-c5ccc6ccccc6c5)c5ccccc45)c3)cc(-n3c4ccccc4c4c5ccccc5ccc43)c2)cc1 ; c1ccc(-c2ccc(-c3cccc(N(c4cccc(-c5cccc6ccccc56)c4)c4ccc(-c5ccc6ccccc6c5)c5ccccc45)c3)cc2-n2c3ccccc3c3c4ccccc4ccc32)cc1 ; c1cc(-c2cccc(-n3c4ccccc4c4cc5ccccc5cc43)c2)cc(N(c2ccccc2-c2cccc3ccccc23)c2ccc(-c3ccc4ccccc4c3)c3ccccc23)c1] 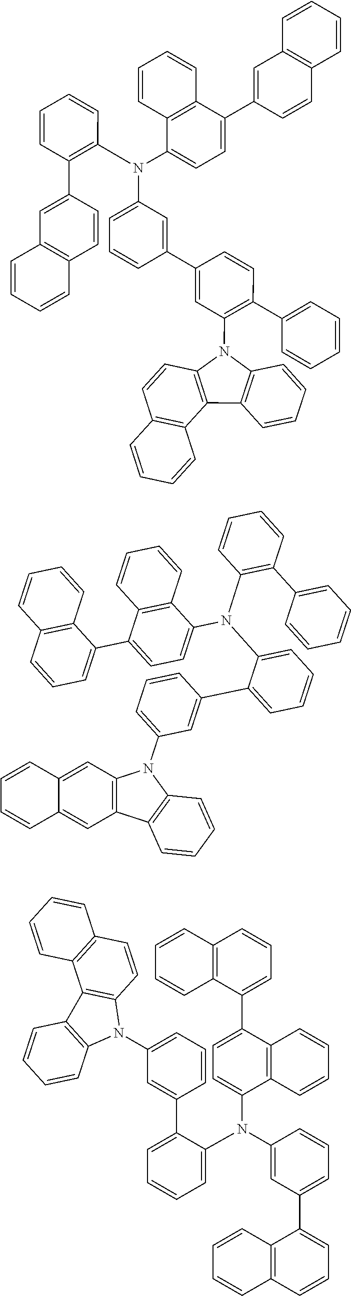 [SMILES: c1ccc(-c2ccc(-c3cccc(N(c4ccccc4-c4ccc5ccccc5c4)c4ccc(-c5ccc6ccccc6c5)c5ccccc45)c3)cc2-n2c3ccccc3c3c4ccccc4ccc32)cc1 ; c1ccc(-c2ccccc2N(c2ccccc2-c2cccc(-n3c4ccccc4c4cc5ccccc5cc43)c2)c2ccc(-c3cccc4ccccc34)c3ccccc23)cc1 ; c1cc(-c2cccc3ccccc23)cc(N(c2ccccc2-c2cccc(-n3c4ccccc4c4c5ccccc5ccc43)c2)c2ccc(-c3cccc4ccccc34)c3ccccc23)c1] 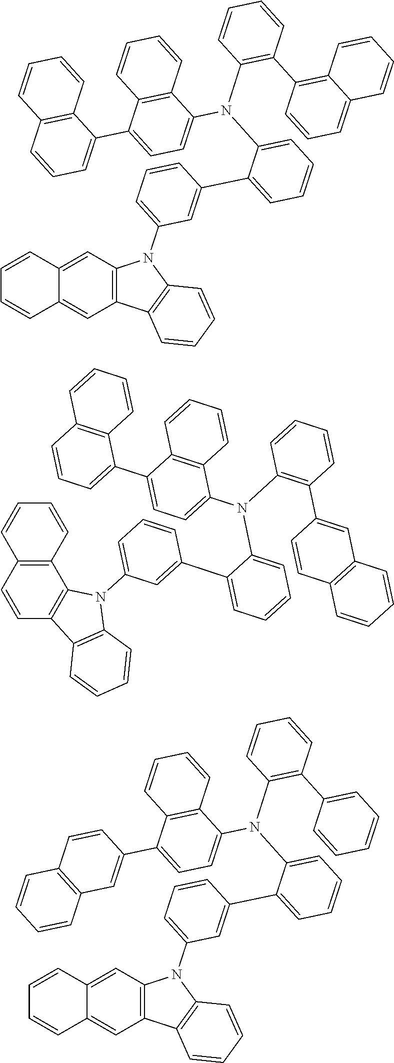 [SMILES: c1cc(-c2ccccc2N(c2ccccc2-c2cccc3ccccc23)c2ccc(-c3cccc4ccccc34)c3ccccc23)cc(-n2c3ccccc3c3cc4ccccc4cc32)c1 ; c1cc(-c2ccccc2N(c2ccccc2-c2ccc3ccccc3c2)c2ccc(-c3cccc4ccccc34)c3ccccc23)cc(-n2c3ccccc3c3ccc4ccccc4c32)c1 ; c1ccc(-c2ccccc2N(c2ccccc2-c2cccc(-n3c4ccccc4c4cc5ccccc5cc43)c2)c2ccc(-c3ccc4ccccc4c3)c3ccccc23)cc1] 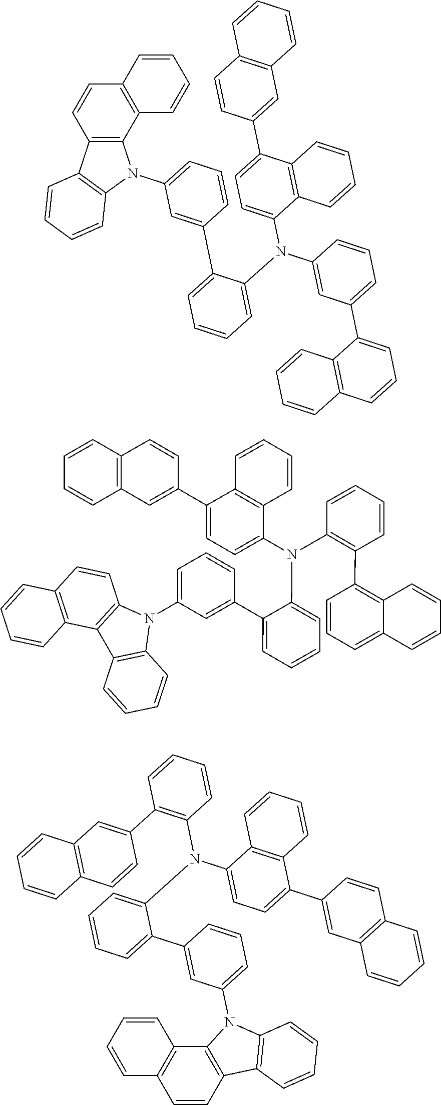 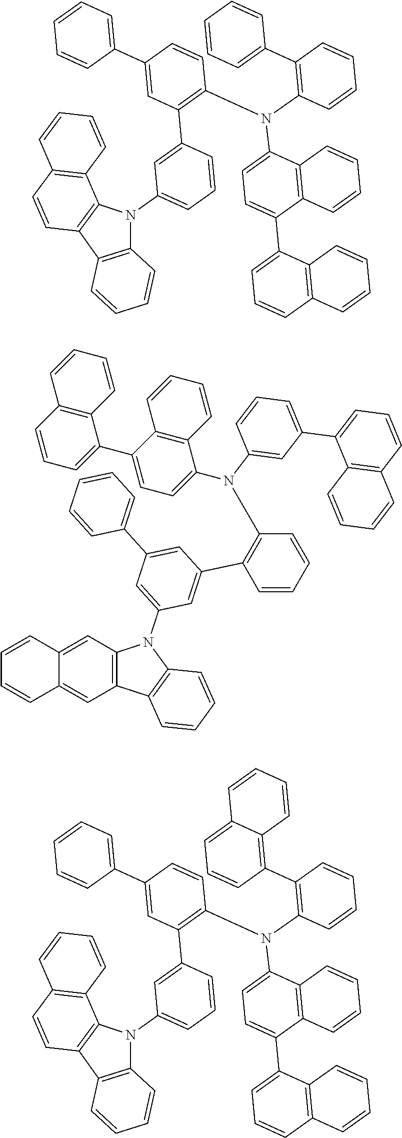 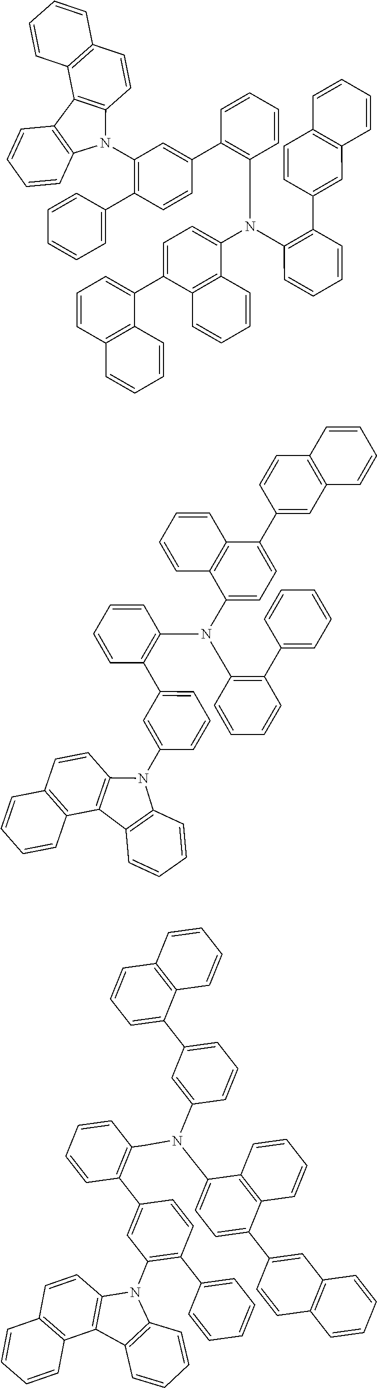 [SMILES: c1ccc(-c2ccc(-c3ccccc3CN(c3ccccc3-c3ccc4ccccc4c3)c3ccc(-c4cccc5ccccc45)c4ccccc34)cc2-n2c3ccccc3c3c4ccccc4ccc32)cc1 ; c1ccc(-c2ccccc2N(Cc2ccccc2-c2cccc(-n3c4ccccc4c4c5ccccc5ccc43)c2)c2ccc(-c3ccc4ccccc4c3)c3ccccc23)cc1 ; c1ccc(-c2ccc(-c3ccccc3CN(c3cccc(-c4cccc5ccccc45)c3)c3ccc(-c4ccc5ccccc5c4)c4ccccc34)cc2-n2c3ccccc3c3c4ccccc4ccc32)cc1] 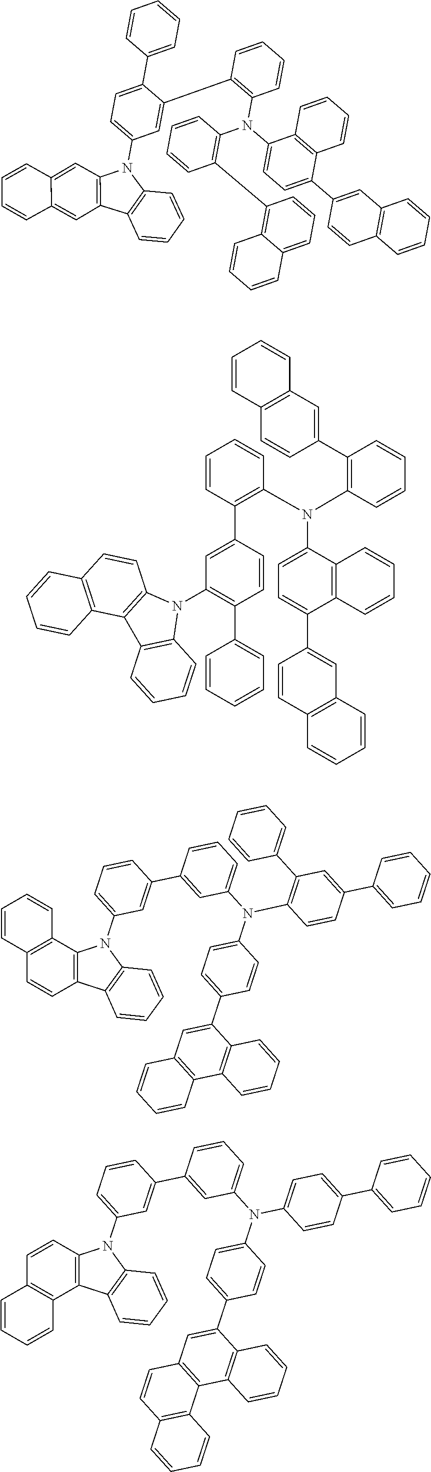 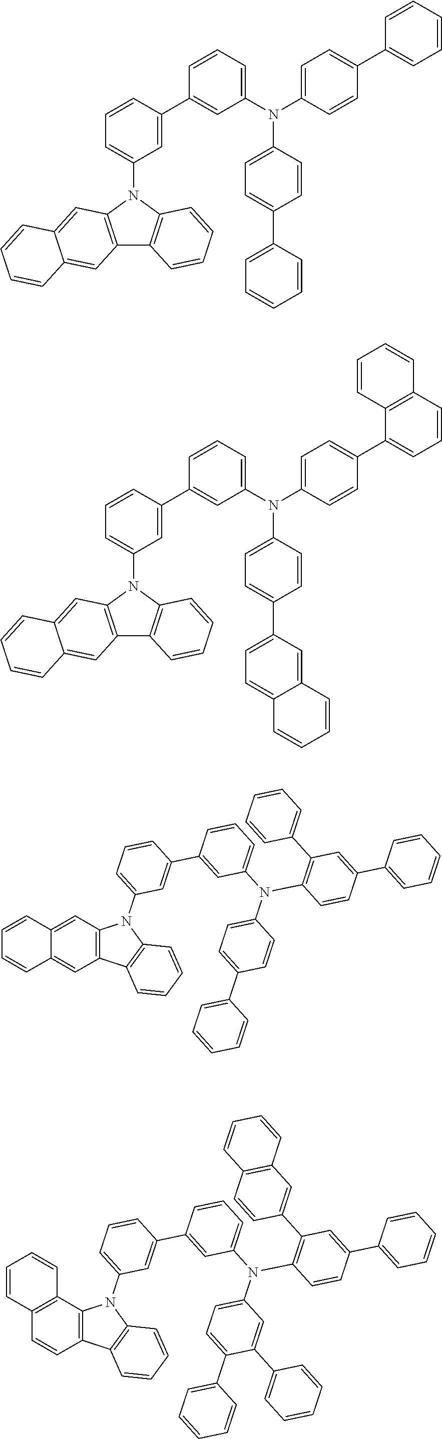 [SMILES: c1ccc(-c2ccc(N(c3ccc(-c4ccccc4)cc3)c3cccc(-c4cccc(-n5c6ccccc6c6cc7ccccc7cc65)c4)c3)cc2)cc1 ; c1cc(-c2cccc(-n3c4ccccc4c4cc5ccccc5cc43)c2)cc(N(c2ccc(-c3ccc4ccccc4c3)cc2)c2ccc(-c3cccc4ccccc34)cc2)c1 ; c1ccc(-c2ccc(N(c3cccc(-c4cccc(-n5c6ccccc6c6cc7ccccc7cc65)c4)c3)c3ccc(-c4ccccc4)cc3-c3ccccc3)cc2)cc1 ; c1ccc(-c2ccc(N(c3cccc(-c4cccc(-n5c6ccccc6c6ccc7ccccc7c65)c4)c3)c3ccc(-c4ccccc4)c(-c4ccccc4)c3)c(-c3ccc4ccccc4c3)c2)cc1] 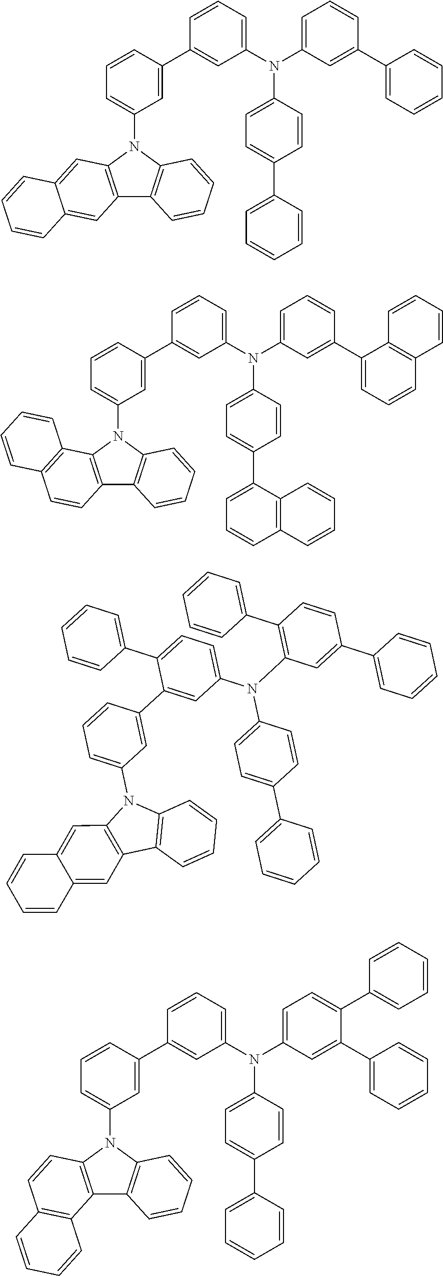 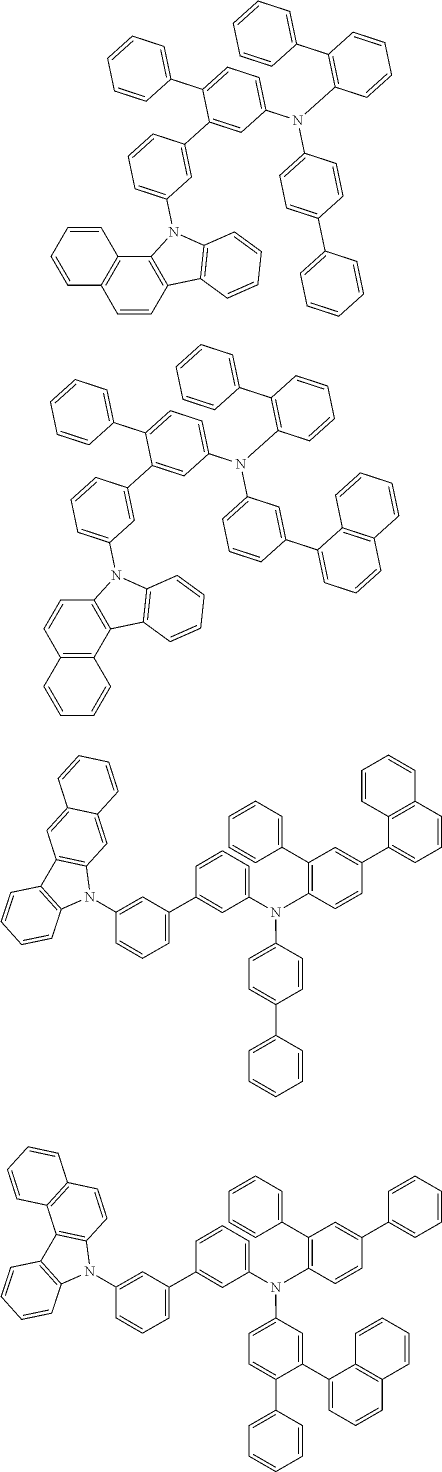 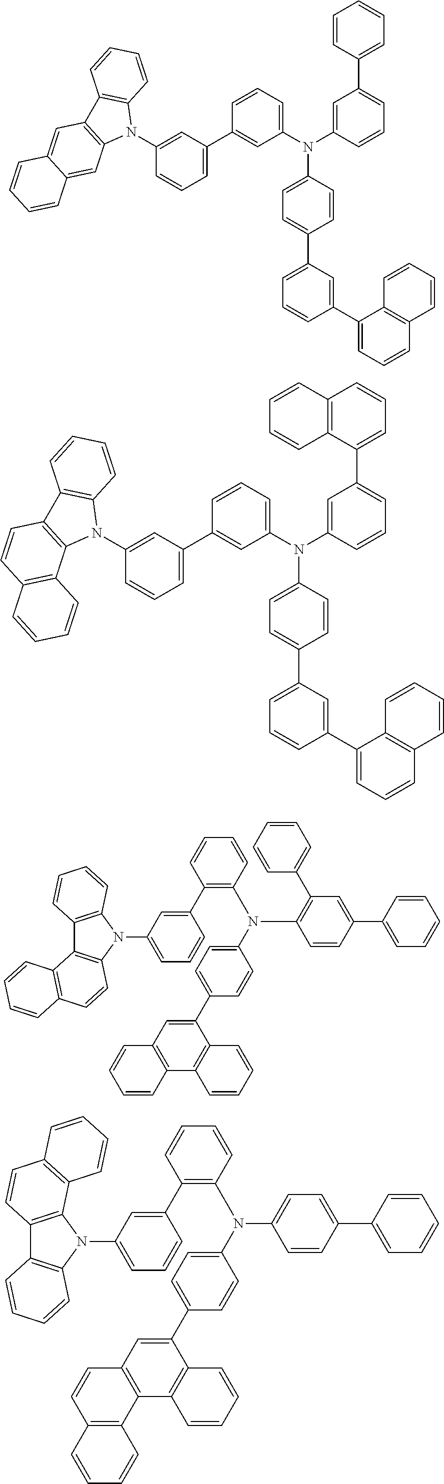 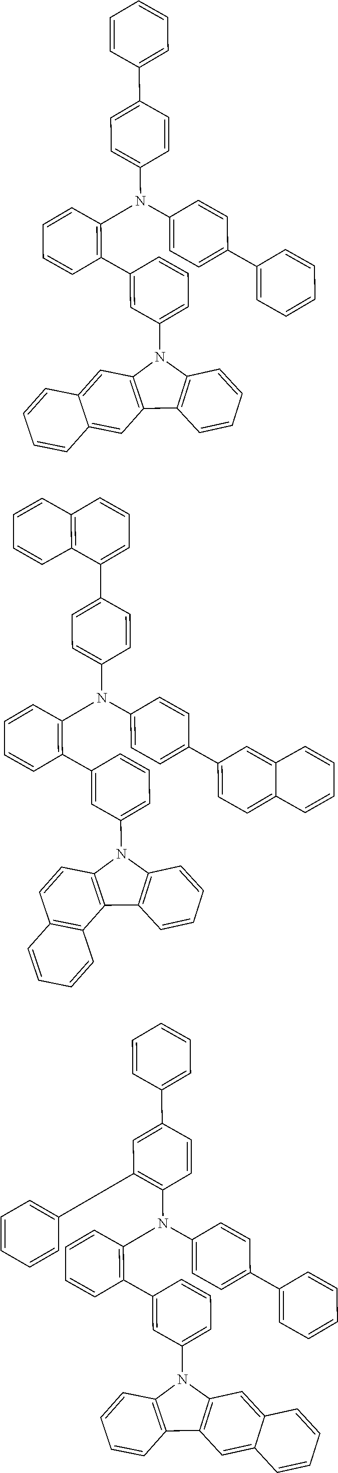 [SMILES: c1ccc(-c2ccc(N(c3ccc(-c4ccccc4)cc3)c3ccccc3-c3cccc(-n4c5ccccc5c5cc6ccccc6cc54)c3)cc2)cc1 ; c1cc(-c2ccccc2N(c2ccc(-c3ccc4ccccc4c3)cc2)c2ccc(-c3cccc4ccccc34)cc2)cc(-n2c3ccccc3c3c4ccccc4ccc32)c1 ; c1ccc(-c2ccc(N(c3ccccc3-c3cccc(-n4c5ccccc5c5cc6ccccc6cc54)c3)c3ccc(-c4ccccc4)cc3-c3ccccc3)cc2)cc1] 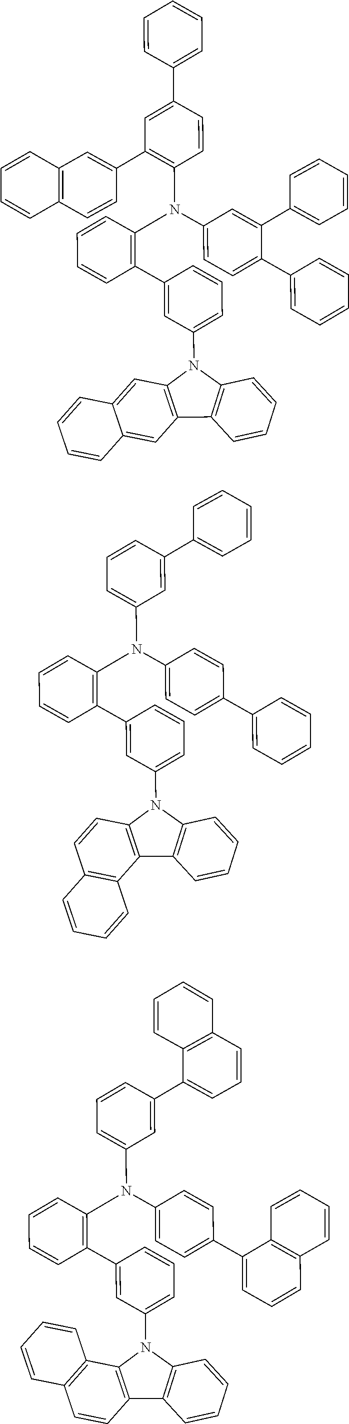 [SMILES: c1ccc(-c2ccc(N(c3ccc(-c4ccccc4)c(-c4ccccc4)c3)c3ccccc3-c3cccc(-n4c5ccccc5c5cc6ccccc6cc54)c3)c(-c3ccc4ccccc4c3)c2)cc1 ; c1ccc(-c2ccc(N(c3cccc(-c4ccccc4)c3)c3ccccc3-c3cccc(-n4c5ccccc5c5c6ccccc6ccc54)c3)cc2)cc1 ; c1cc(-c2cccc3ccccc23)cc(N(c2ccc(-c3cccc4ccccc34)cc2)c2ccccc2-c2cccc(-n3c4ccccc4c4ccc5ccccc5c43)c2)c1] 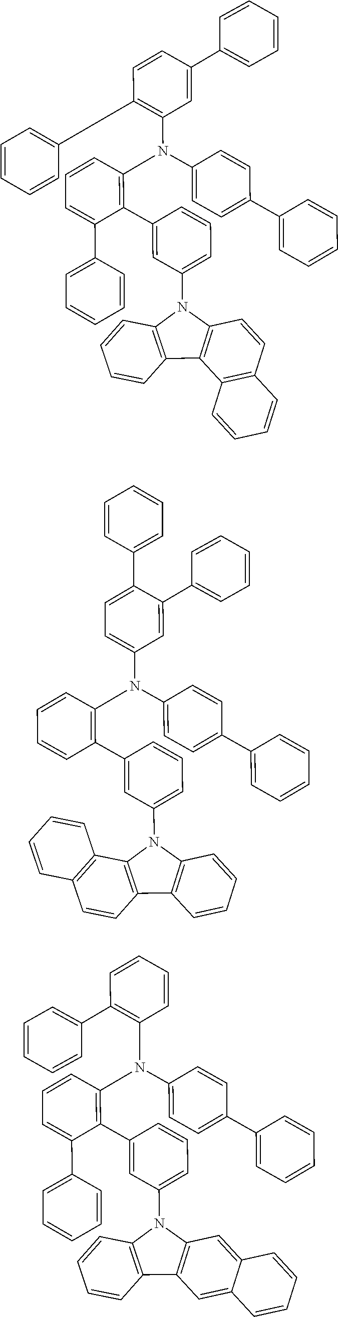 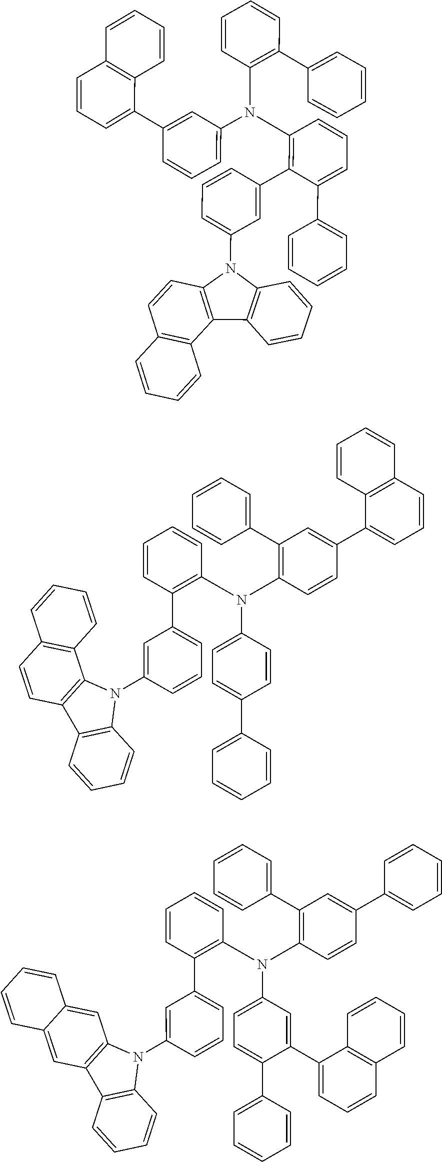 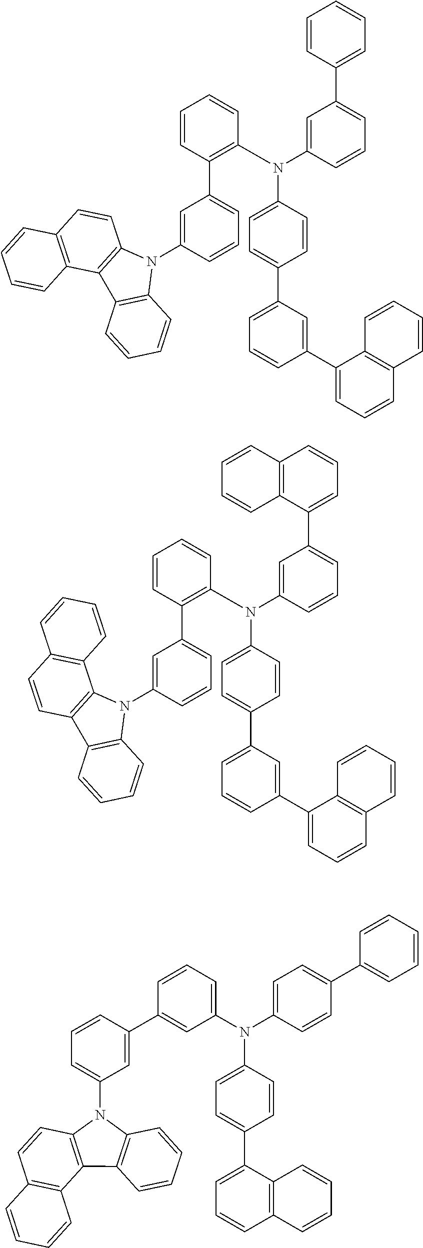 [SMILES: c1ccc(-c2cccc(N(c3ccc(-c4cccc(-c5cccc6ccccc56)c4)cc3)c3ccccc3-c3cccc(-n4c5ccccc5c5c6ccccc6ccc54)c3)c2)cc1 ; c1cc(-c2ccc(N(c3cccc(-c4cccc5ccccc45)c3)c3ccccc3-c3cccc(-n4c5ccccc5c5ccc6ccccc6c54)c3)cc2)cc(-c2cccc3ccccc23)c1 ; c1ccc(-c2ccc(N(c3ccc(-c4cccc5ccccc45)cc3)c3cccc(-c4cccc(-n5c6ccccc6c6c7ccccc7ccc65)c4)c3)cc2)cc1] 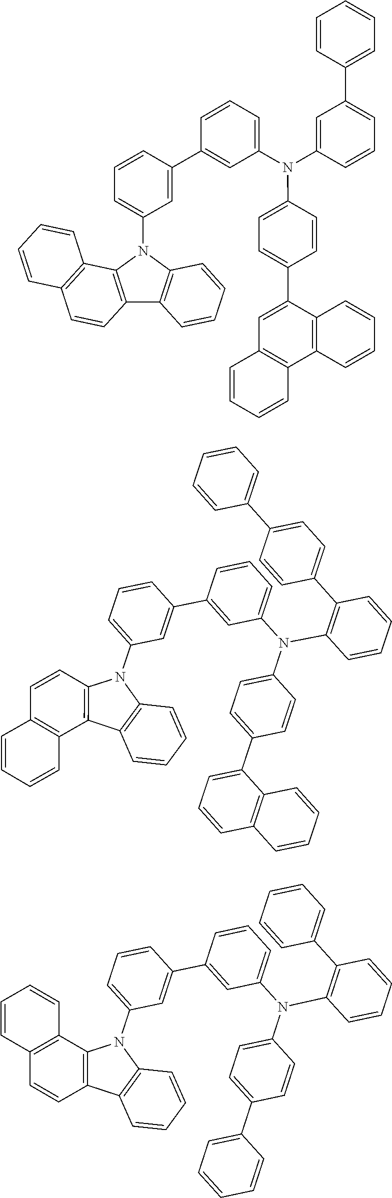 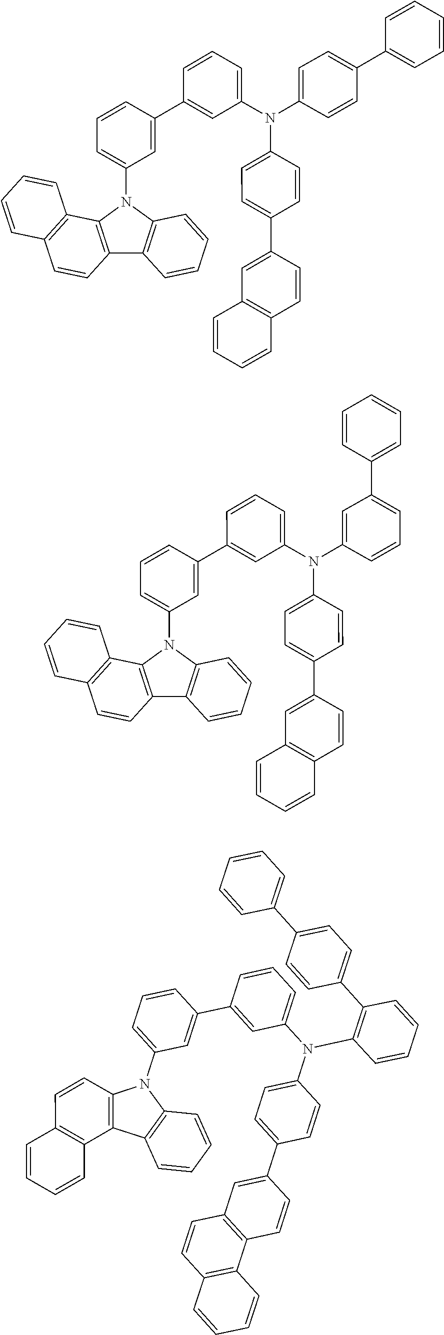 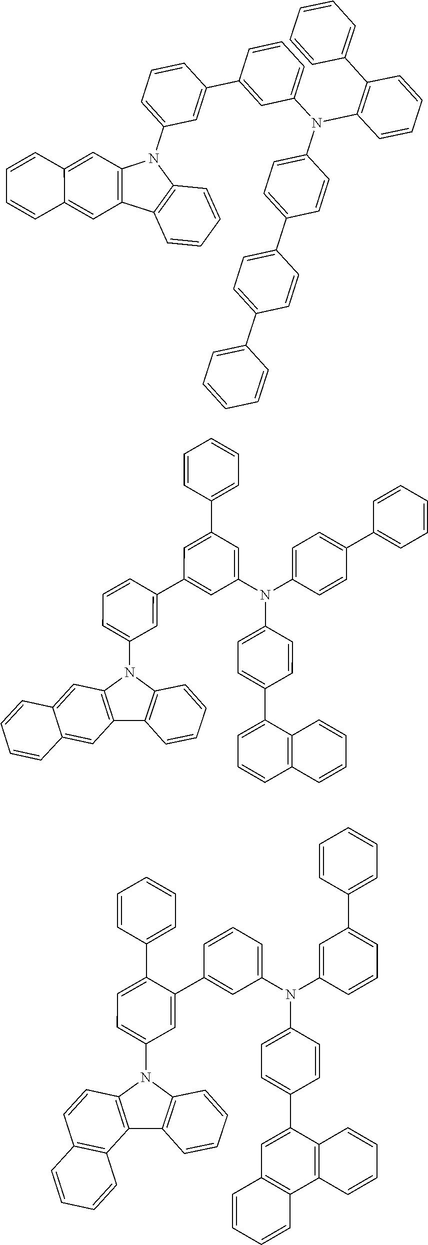 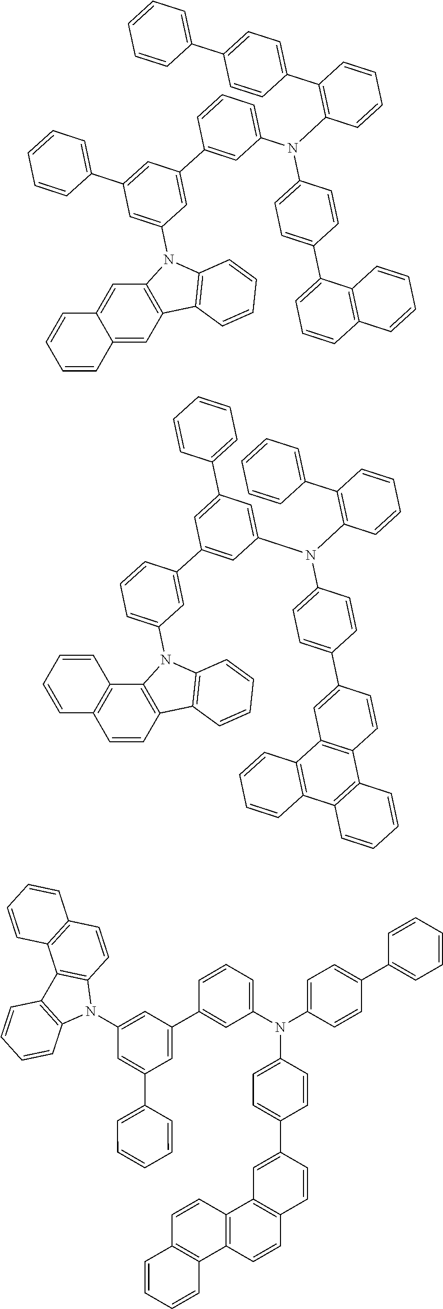 [SMILES: c1ccc(-c2ccc(-c3ccccc3N(c3ccc(-c4cccc5ccccc45)cc3)c3cccc(-c4cc(-c5ccccc5)cc(-n5c6ccccc6c6cc7ccccc7cc65)c4)c3)cc2)cc1 ; c1ccc(-c2cc(-c3cccc(-n4c5ccccc5c5ccc6ccccc6c54)c3)cc(N(c3ccc(-c4ccc5c6ccccc6c6ccccc6c5c4)cc3)c3ccccc3-c3ccccc3)c2)cc1 ; c1ccc(-c2ccc(N(c3ccc(-c4ccc5ccc6c7ccccc7ccc6c5c4)cc3)c3cccc(-c4cc(-c5ccccc5)cc(-n5c6ccccc6c6c7ccccc7ccc65)c4)c3)cc2)cc1] 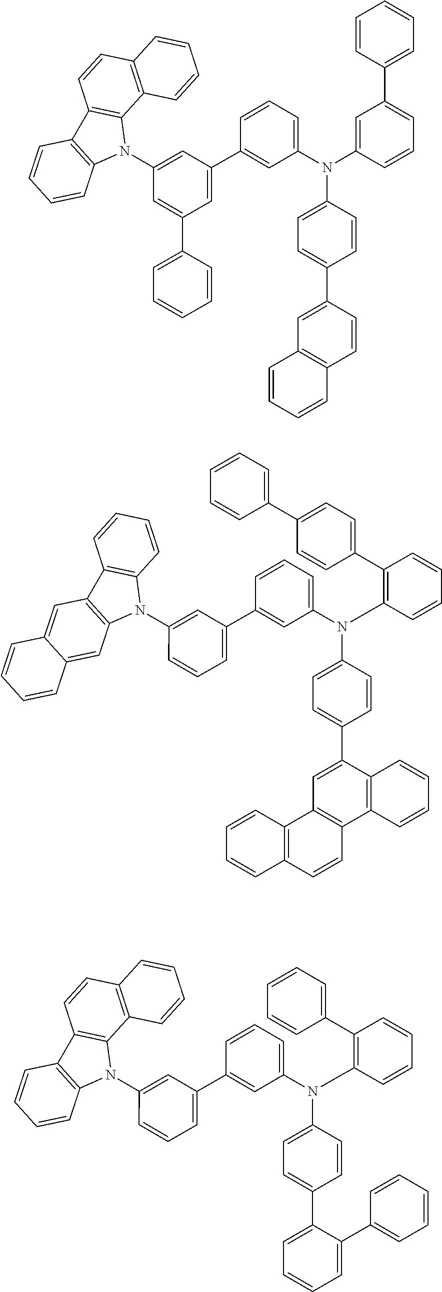 [SMILES: c1ccc(-c2cccc(N(c3ccc(-c4ccc5ccccc5c4)cc3)c3cccc(-c4cc(-c5ccccc5)cc(-n5c6ccccc6c6ccc7ccccc7c65)c4)c3)c2)cc1 ; c1ccc(-c2ccc(-c3ccccc3N(c3ccc(-c4cc5c6ccccc6ccc5c5ccccc45)cc3)c3cccc(-c4cccc(-n5c6ccccc6c6cc7ccccc7cc65)c4)c3)cc2)cc1 ; c1ccc(-c2ccccc2-c2ccc(N(c3cccc(-c4cccc(-n5c6ccccc6c6ccc7ccccc7c65)c4)c3)c3ccccc3-c3ccccc3)cc2)cc1] 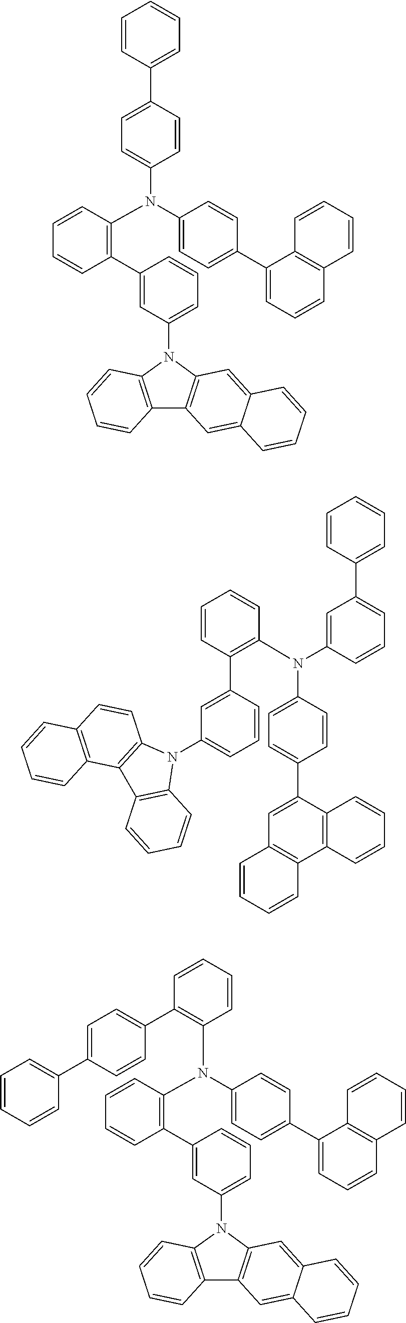 [SMILES: c1ccc(-c2ccc(N(c3ccc(-c4cccc5ccccc45)cc3)c3ccccc3-c3cccc(-n4c5ccccc5c5cc6ccccc6cc54)c3)cc2)cc1 ; c1ccc(-c2cccc(N(c3ccc(-c4cc5ccccc5c5ccccc45)cc3)c3ccccc3-c3cccc(-n4c5ccccc5c5c6ccccc6ccc54)c3)c2)cc1 ; c1ccc(-c2ccc(-c3ccccc3N(c3ccc(-c4cccc5ccccc45)cc3)c3ccccc3-c3cccc(-n4c5ccccc5c5cc6ccccc6cc54)c3)cc2)cc1] 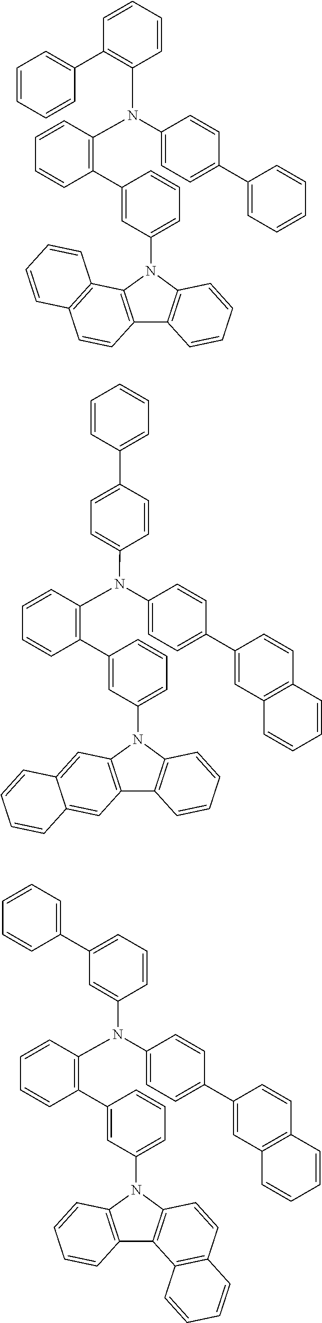 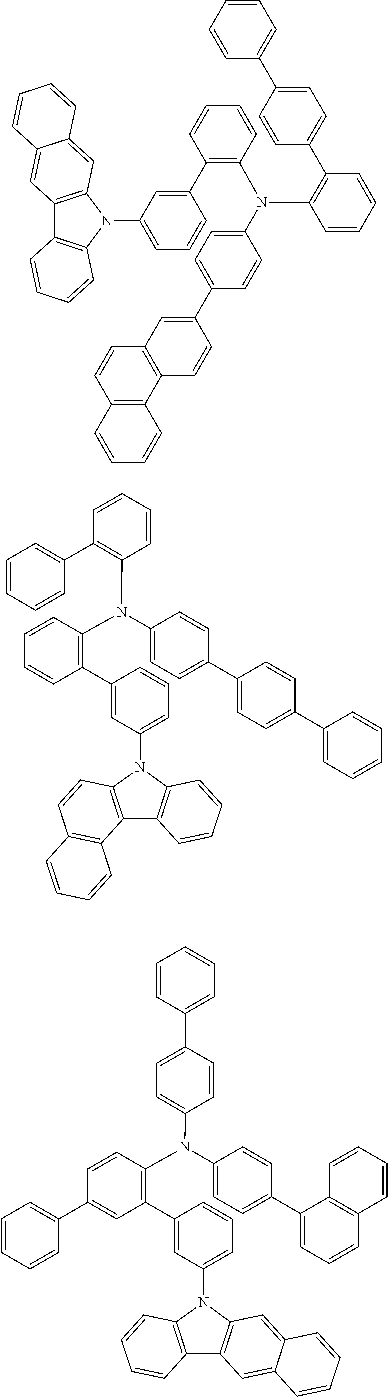 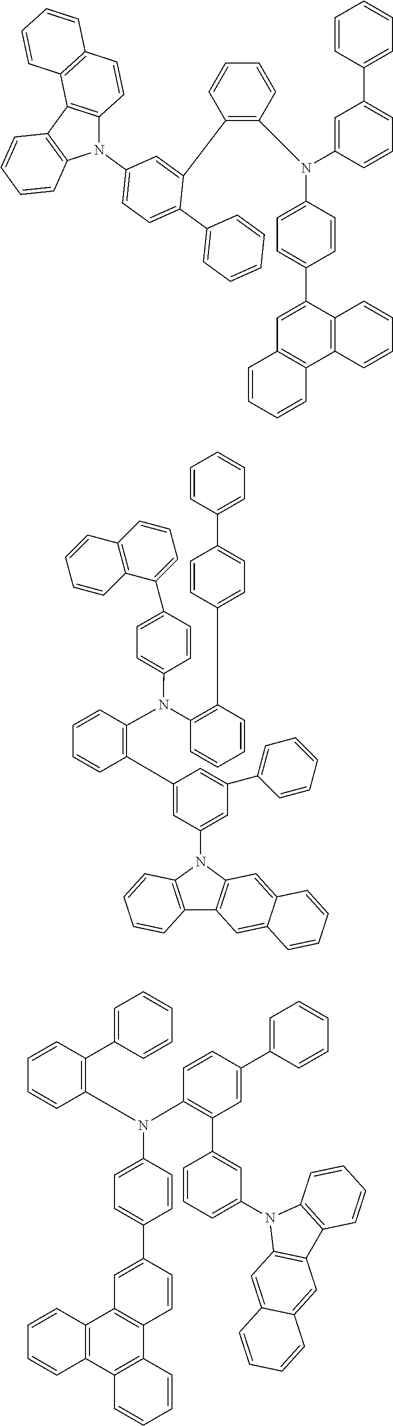 [SMILES: c1ccc(-c2cccc(N(Cc3ccccc3-c3cc(-n4c5ccccc5c5c6ccccc6ccc54)ccc3-c3ccccc3)c3ccc(-c4cc5ccccc5c5ccccc45)cc3)c2)cc1 ; c1ccc(-c2ccc(-c3ccccc3N(c3ccc(-c4cccc5ccccc45)cc3)c3ccccc3-c3cc(-c4ccccc4)cc(-n4c5ccccc5c5cc6ccccc6cc54)c3)cc2)cc1 ; c1ccc(-c2ccc(N(Cc3ccccc3-c3ccccc3)c3ccc(-c4ccc5c6ccccc6c6ccccc6c5c4)cc3)c(-c3cccc(-n4c5ccccc5c5cc6ccccc6cc54)c3)c2)cc1] 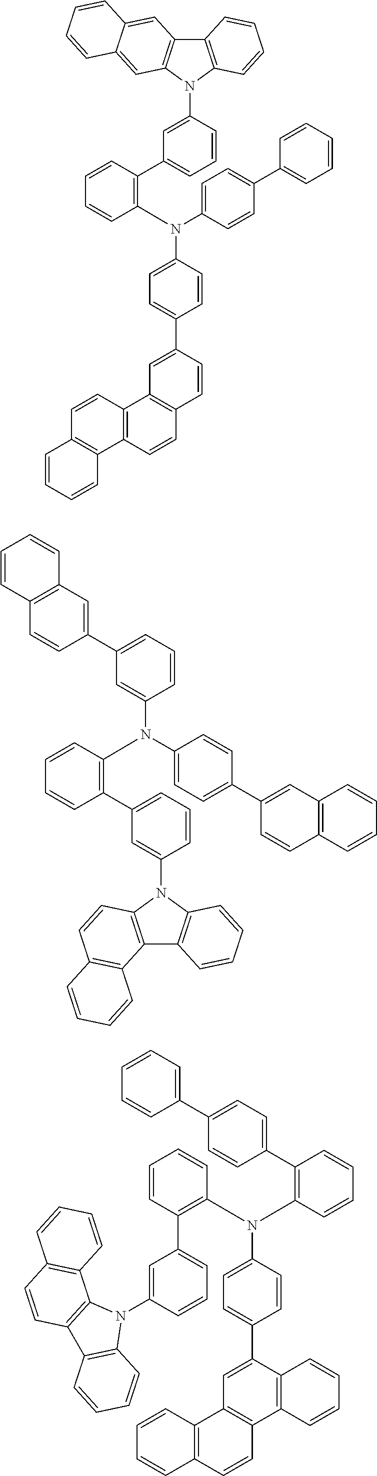 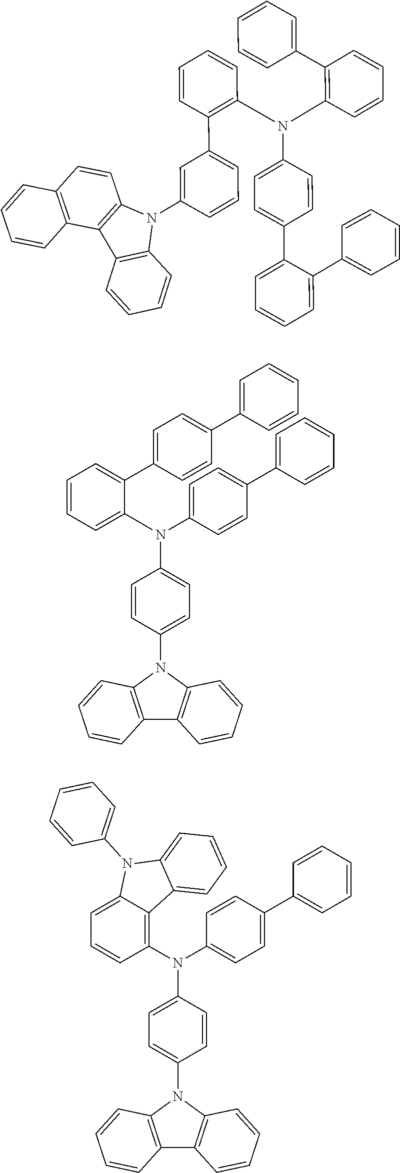 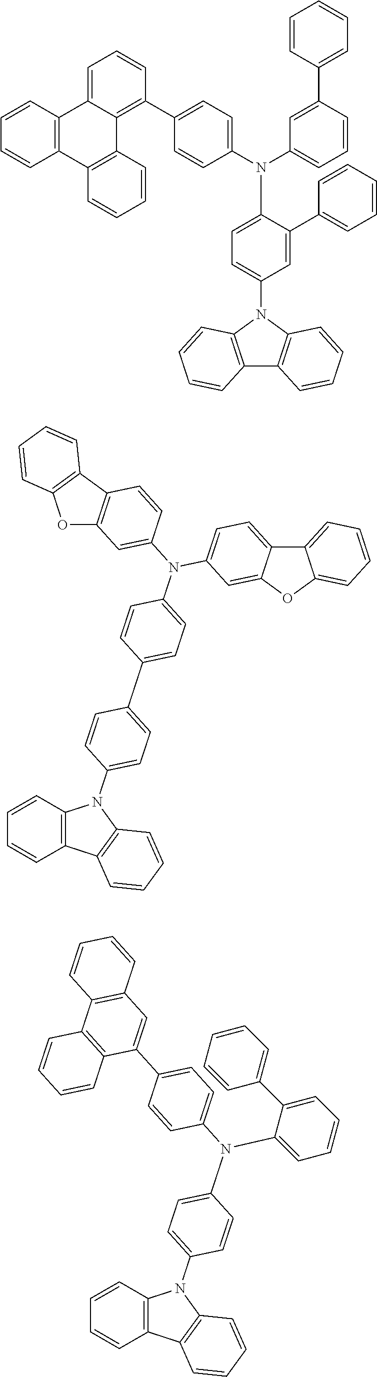 [SMILES: c1ccc(-c2cccc(N(c3ccc(-c4cccc5c6ccccc6c6ccccc6c45)cc3)c3ccc(-n4c5ccccc5c5ccccc54)cc3-c3ccccc3)c2)cc1 ; c1ccc2c(c1)oc1cc(N(c3ccc(-c4ccc(-n5c6ccccc6c6ccccc65)cc4)cc3)c3ccc4c(c3)oc3ccccc34)ccc12 ; c1ccc(-c2ccccc2N(c2ccc(-c3cc4ccccc4c4ccccc34)cc2)c2ccc(-n3c4ccccc4c4ccccc43)cc2)cc1] 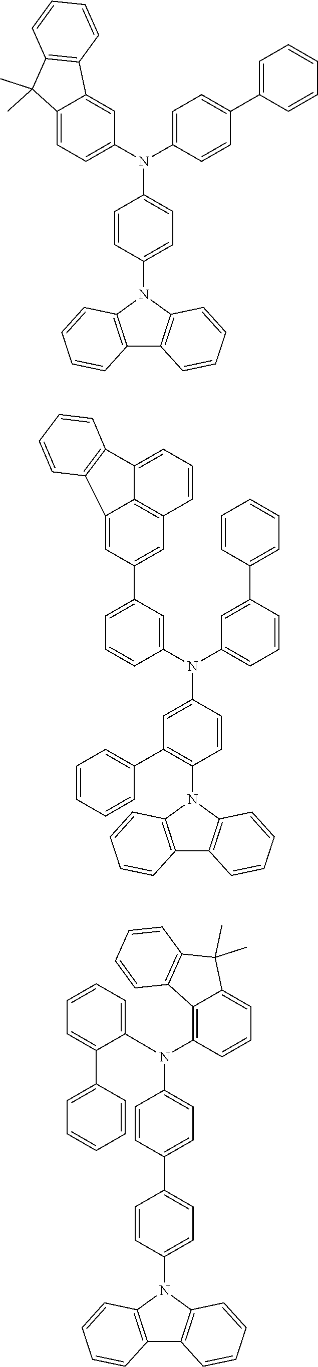 [SMILES: CC1(C)c2ccccc2-c2cc(N(c3ccc(-c4ccccc4)cc3)c3ccc(-n4c5ccccc5c5ccccc54)cc3)ccc21 ; c1ccc(-c2cccc(N(c3cccc(-c4cc5c6c(cccc6c4)-c4ccccc4-5)c3)c3ccc(-n4c5ccccc5c5ccccc54)c(-c4ccccc4)c3)c2)cc1 ; CC1(C)c2ccccc2-c2c(N(c3ccc(-c4ccc(-n5c6ccccc6c6ccccc65)cc4)cc3)c3ccccc3-c3ccccc3)cccc21] 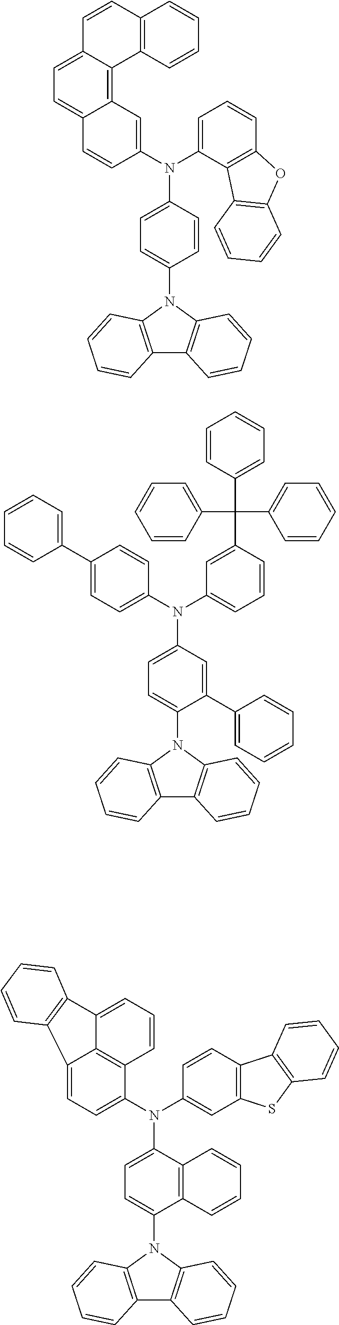 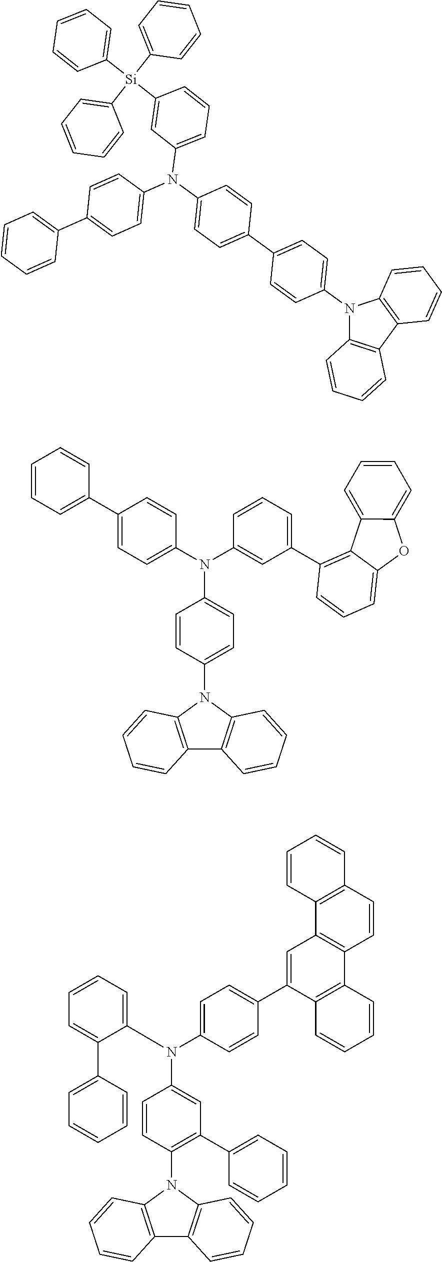 [SMILES: c1ccc(-c2ccc(N(c3ccc(-c4ccc(-n5c6ccccc6c6ccccc65)cc4)cc3)c3cccc([Si](c4ccccc4)(c4ccccc4)c4ccccc4)c3)cc2)cc1 ; c1ccc(-c2ccc(N(c3ccc(-n4c5ccccc5c5ccccc54)cc3)c3cccc(-c4cccc5oc6ccccc6c45)c3)cc2)cc1 ; c1ccc(-c2ccccc2N(c2ccc(-c3cc4c5ccccc5ccc4c4ccccc34)cc2)c2ccc(-n3c4ccccc4c4ccccc43)c(-c3ccccc3)c2)cc1] 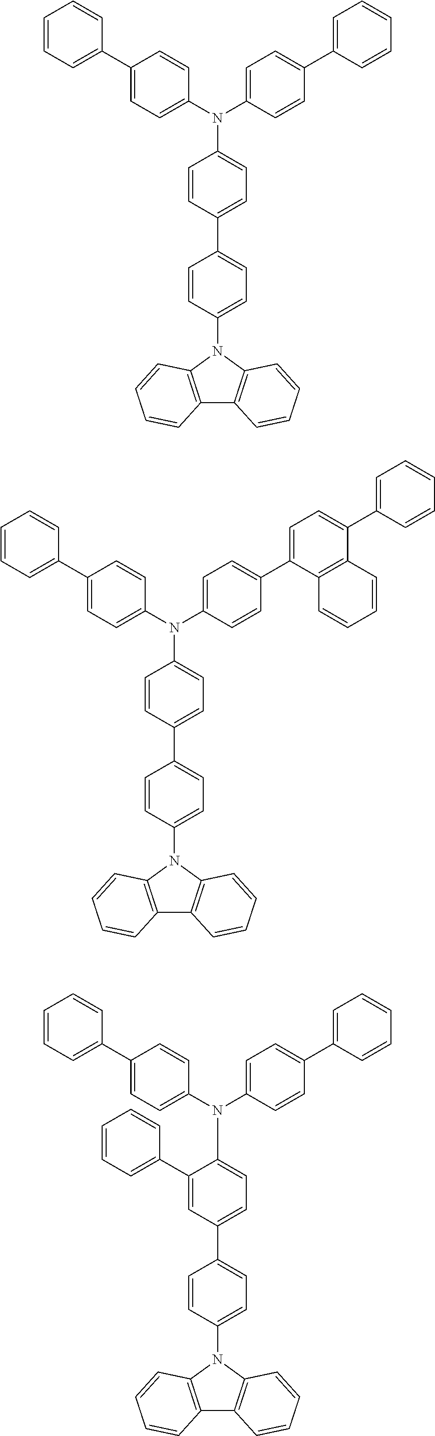 [SMILES: c1ccc(-c2ccc(N(c3ccc(-c4ccccc4)cc3)c3ccc(-c4ccc(-n5c6ccccc6c6ccccc65)cc4)cc3)cc2)cc1 ; c1ccc(-c2ccc(N(c3ccc(-c4ccc(-n5c6ccccc6c6ccccc65)cc4)cc3)c3ccc(-c4ccc(-c5ccccc5)c5ccccc45)cc3)cc2)cc1 ; c1ccc(-c2ccc(N(c3ccc(-c4ccccc4)cc3)c3ccc(-c4ccc(-n5c6ccccc6c6ccccc65)cc4)cc3-c3ccccc3)cc2)cc1] 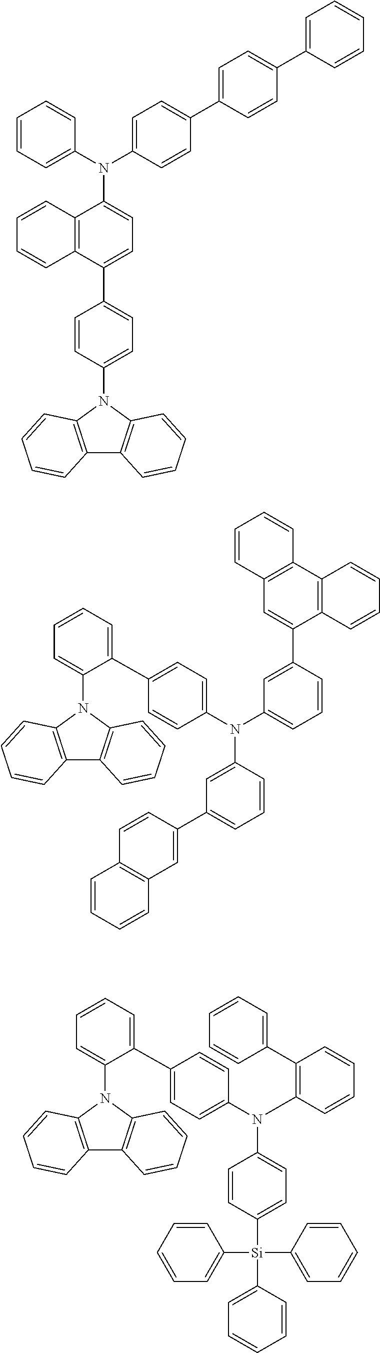 [SMILES: c1ccc(-c2ccc(-c3ccc(N(c4ccccc4)c4ccc(-c5ccc(-n6c7ccccc7c7ccccc76)cc5)c5ccccc45)cc3)cc2)cc1 ; c1cc(-c2ccc3ccccc3c2)cc(N(c2ccc(-c3ccccc3-n3c4ccccc4c4ccccc43)cc2)c2cccc(-c3cc4ccccc4c4ccccc34)c2)c1 ; c1ccc(-c2ccccc2N(c2ccc(-c3ccccc3-n3c4ccccc4c4ccccc43)cc2)c2ccc([Si](c3ccccc3)(c3ccccc3)c3ccccc3)cc2)cc1] 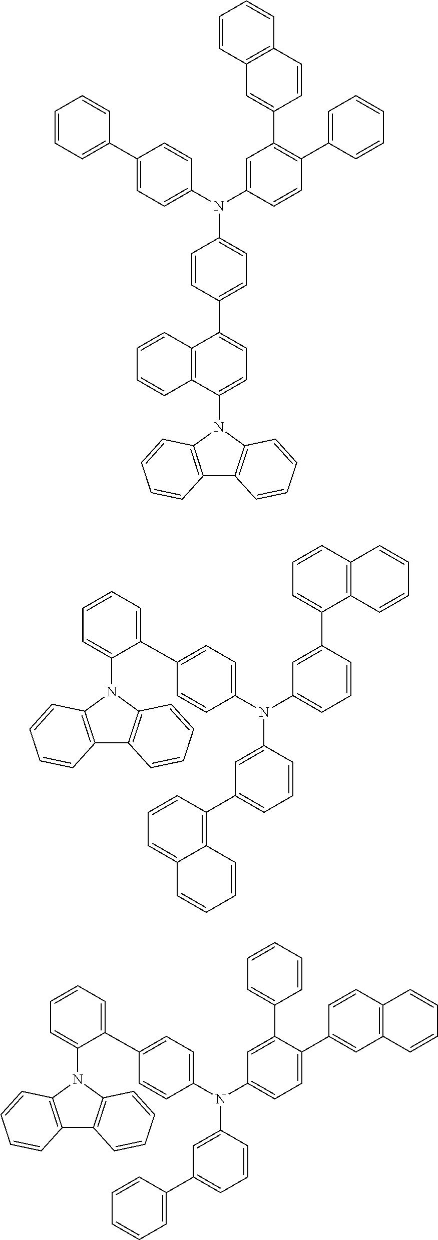 [SMILES: c1ccc(-c2ccc(N(c3ccc(-c4ccc(-n5c6ccccc6c6ccccc65)c5ccccc45)cc3)c3ccc(-c4ccccc4)c(-c4ccc5ccccc5c4)c3)cc2)cc1 ; c1cc(-c2cccc3ccccc23)cc(N(c2ccc(-c3ccccc3-n3c4ccccc4c4ccccc43)cc2)c2cccc(-c3cccc4ccccc34)c2)c1 ; c1ccc(-c2cccc(N(c3ccc(-c4ccccc4-n4c5ccccc5c5ccccc54)cc3)c3ccc(-c4ccc5ccccc5c4)c(-c4ccccc4)c3)c2)cc1] 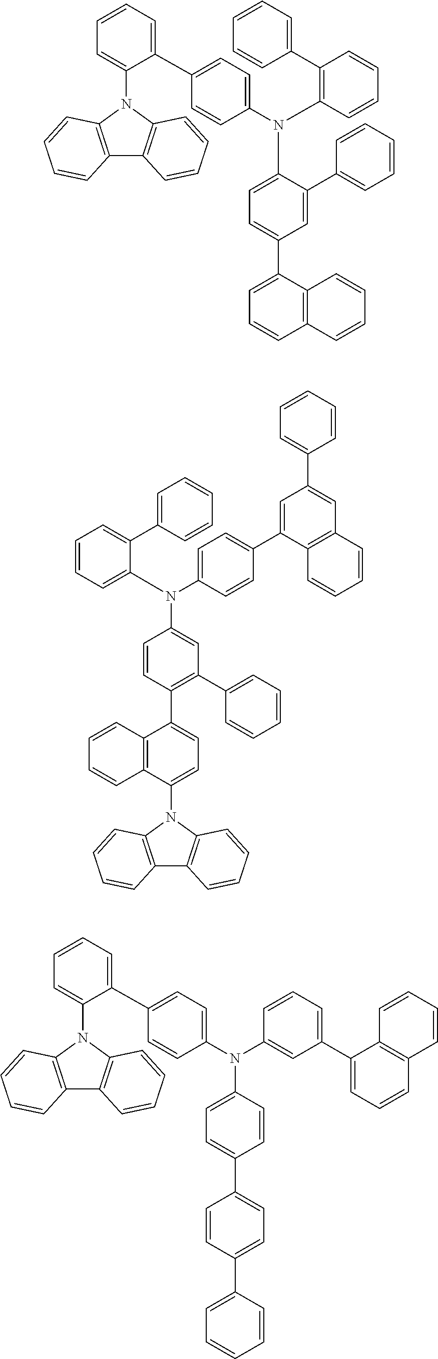 [SMILES: c1ccc(-c2ccccc2N(c2ccc(-c3ccccc3-n3c4ccccc4c4ccccc43)cc2)c2ccc(-c3cccc4ccccc34)cc2-c2ccccc2)cc1 ; c1ccc(-c2cc(-c3ccc(N(c4ccc(-c5ccc(-n6c7ccccc7c7ccccc76)c6ccccc56)c(-c5ccccc5)c4)c4ccccc4-c4ccccc4)cc3)c3ccccc3c2)cc1 ; c1ccc(-c2ccc(-c3ccc(N(c4ccc(-c5ccccc5-n5c6ccccc6c6ccccc65)cc4)c4cccc(-c5cccc6ccccc56)c4)cc3)cc2)cc1] 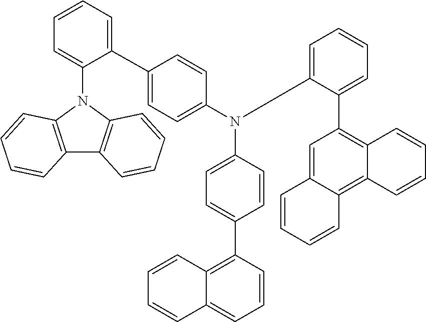 [SMILES: c1ccc(N(c2ccc(-c3ccccc3-n3c4ccccc4c4ccccc43)cc2)c2ccc(-c3cccc4ccccc34)cc2)c(-c2cc3ccccc3c3ccccc23)c1] 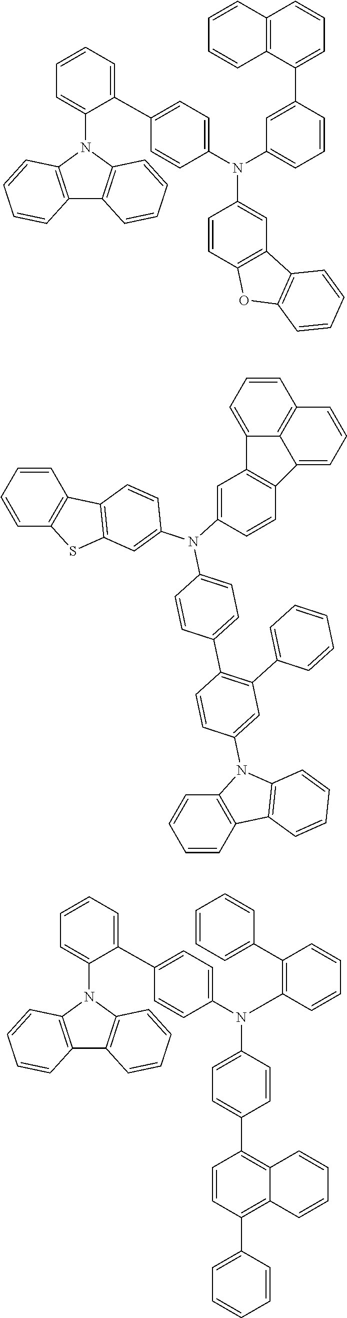 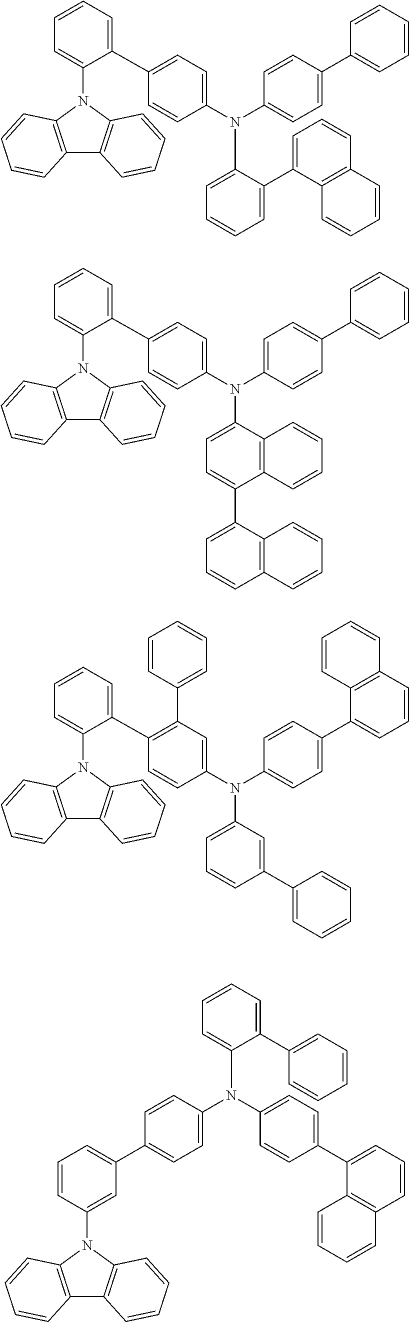 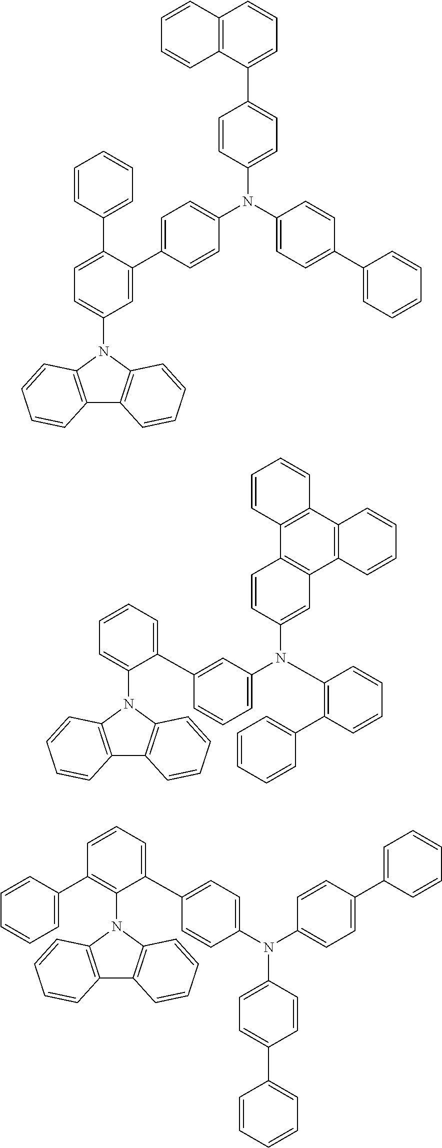 [SMILES: c1ccc(-c2ccc(N(c3ccc(-c4cc(-n5c6ccccc6c6ccccc65)ccc4-c4ccccc4)cc3)c3ccc(-c4cccc5ccccc45)cc3)cc2)cc1 ; c1ccc(-c2ccccc2N(c2cccc(-c3ccccc3-n3c4ccccc4c4ccccc43)c2)c2ccc3c4ccccc4c4ccccc4c3c2)cc1 ; c1ccc(-c2ccc(N(c3ccc(-c4ccccc4)cc3)c3ccc(-c4cccc(-c5ccccc5)c4-n4c5ccccc5c5ccccc54)cc3)cc2)cc1] 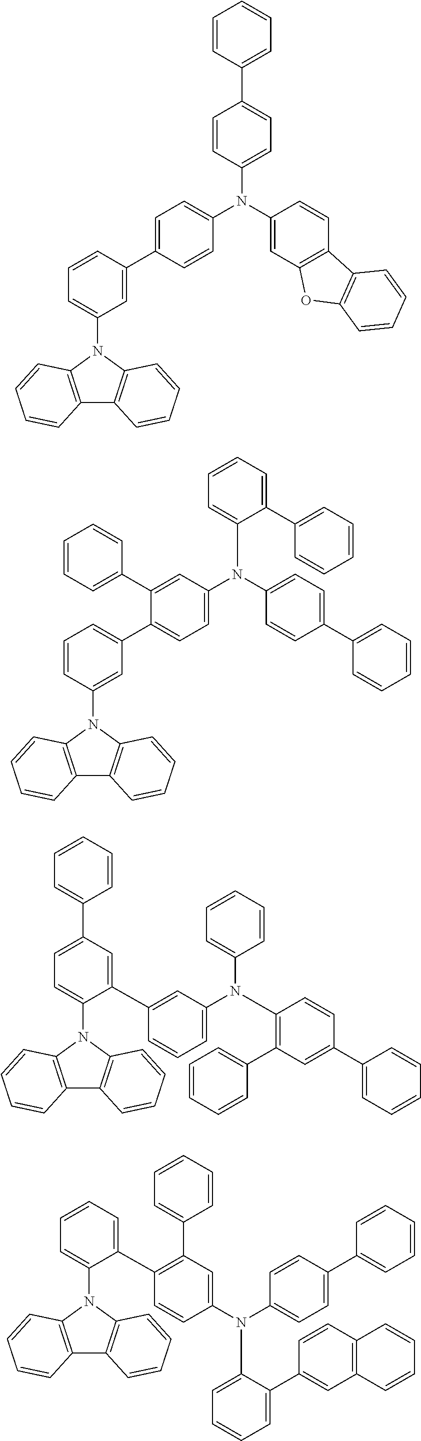 [SMILES: c1ccc(-c2ccc(N(c3ccc(-c4cccc(-n5c6ccccc6c6ccccc65)c4)cc3)c3ccc4c(c3)oc3ccccc34)cc2)cc1 ; c1ccc(-c2ccc(N(c3ccc(-c4cccc(-n5c6ccccc6c6ccccc65)c4)c(-c4ccccc4)c3)c3ccccc3-c3ccccc3)cc2)cc1 ; c1ccc(-c2ccc(N(c3ccccc3)c3cccc(-c4cc(-c5ccccc5)ccc4-n4c5ccccc5c5ccccc54)c3)c(-c3ccccc3)c2)cc1 ; c1ccc(-c2ccc(N(c3ccc(-c4ccccc4-n4c5ccccc5c5ccccc54)c(-c4ccccc4)c3)c3ccccc3-c3ccc4ccccc4c3)cc2)cc1] 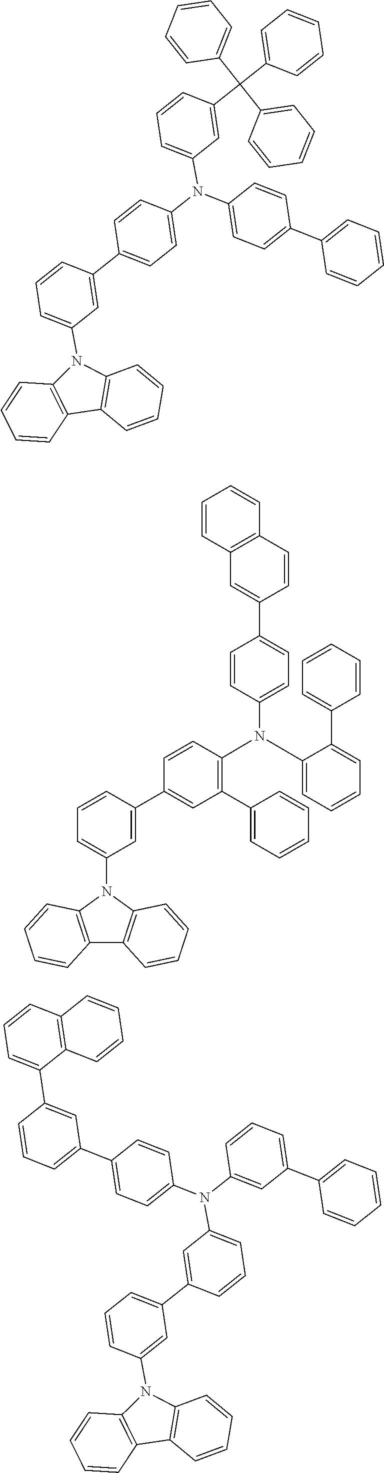 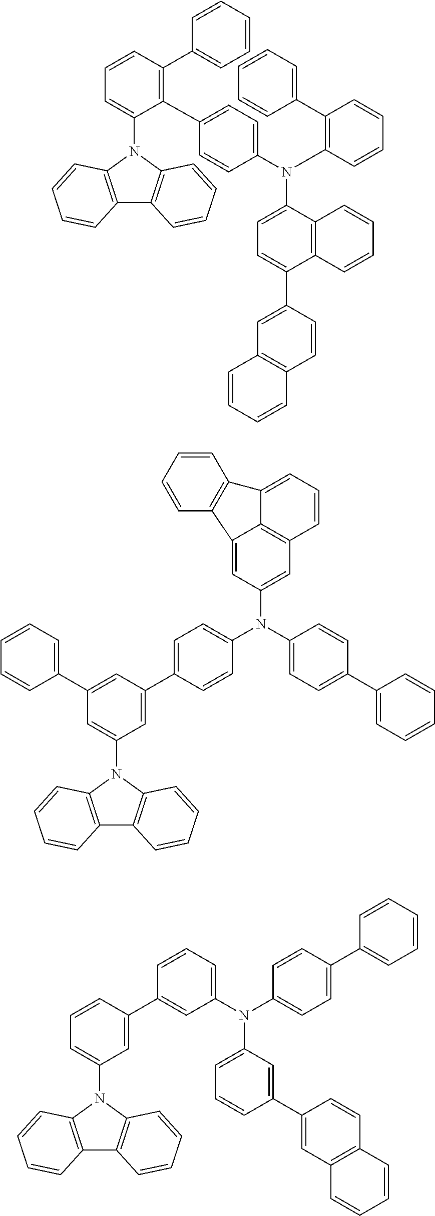 [SMILES: c1ccc(-c2ccccc2N(c2ccc(-c3c(-c4ccccc4)cccc3-n3c4ccccc4c4ccccc43)cc2)c2ccc(-c3ccc4ccccc4c3)c3ccccc23)cc1 ; c1ccc(-c2ccc(N(c3ccc(-c4cc(-c5ccccc5)cc(-n5c6ccccc6c6ccccc65)c4)cc3)c3cc4c5c(cccc5c3)-c3ccccc3-4)cc2)cc1 ; c1ccc(-c2ccc(N(c3cccc(-c4cccc(-n5c6ccccc6c6ccccc65)c4)c3)c3cccc(-c4ccc5ccccc5c4)c3)cc2)cc1] 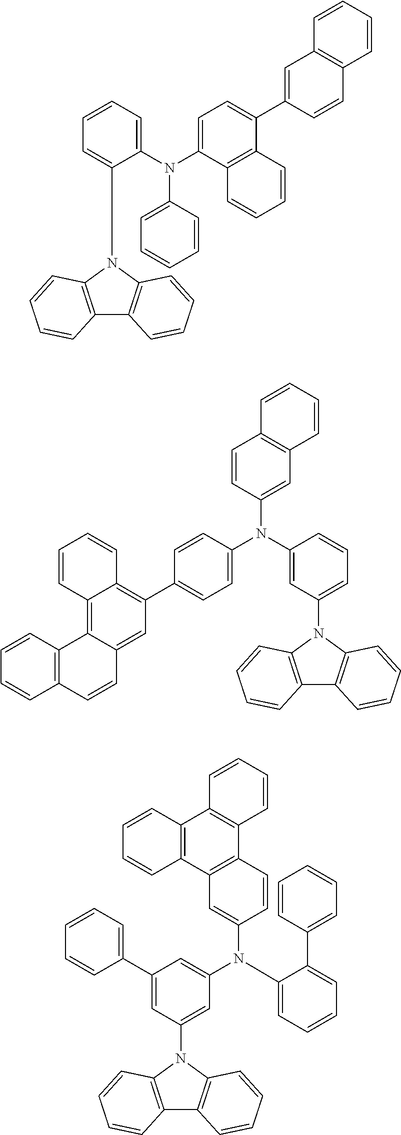 [SMILES: c1ccc(N(c2ccccc2-n2c3ccccc3c3ccccc32)c2ccc(-c3ccc4ccccc4c3)c3ccccc23)cc1 ; c1cc(N(c2ccc(-c3cc4ccc5ccccc5c4c4ccccc34)cc2)c2ccc3ccccc3c2)cc(-n2c3ccccc3c3ccccc32)c1 ; c1ccc(-c2cc(N(c3ccc4c5ccccc5c5ccccc5c4c3)c3ccccc3-c3ccccc3)cc(-n3c4ccccc4c4ccccc43)c2)cc1] 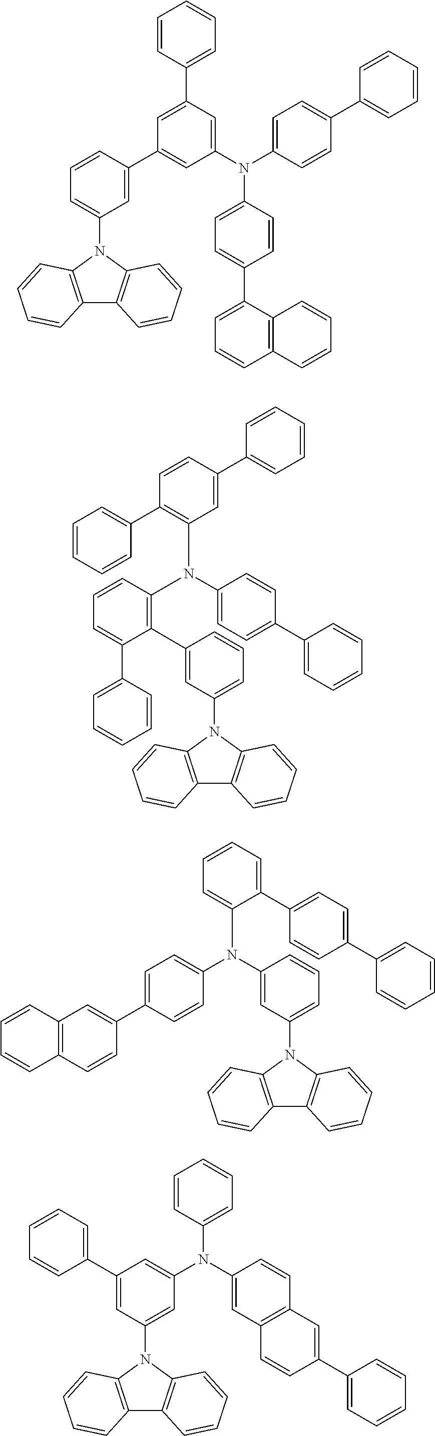 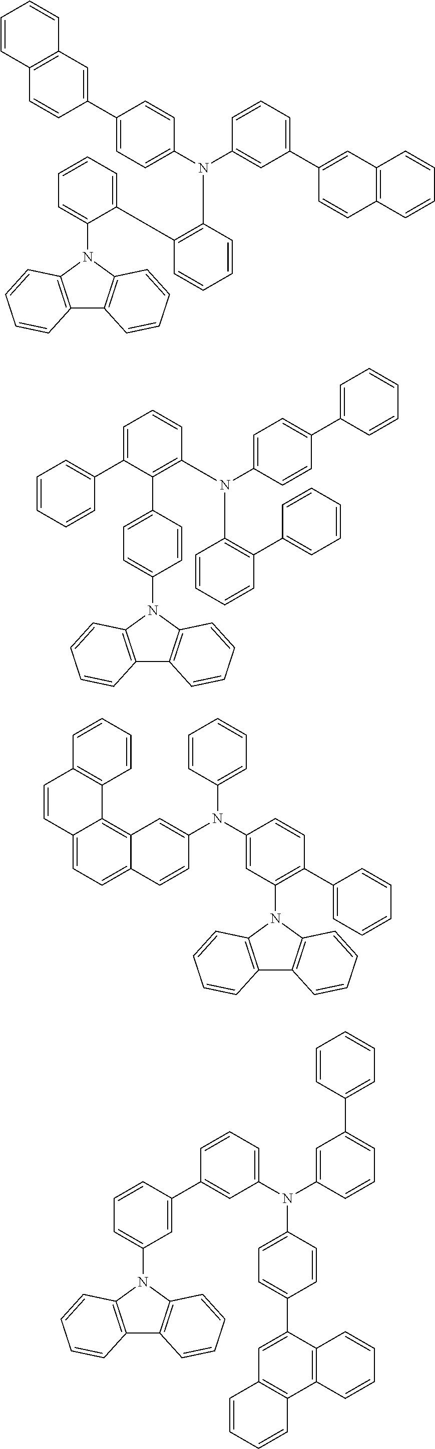 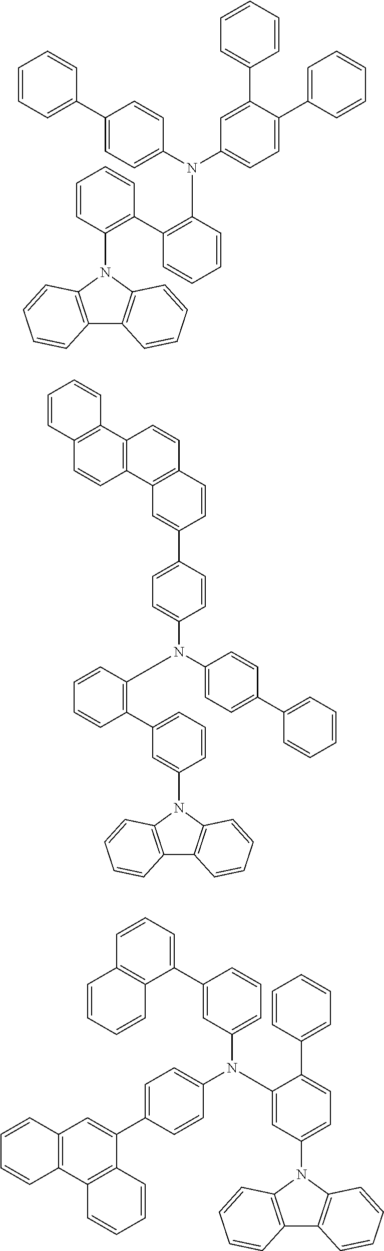 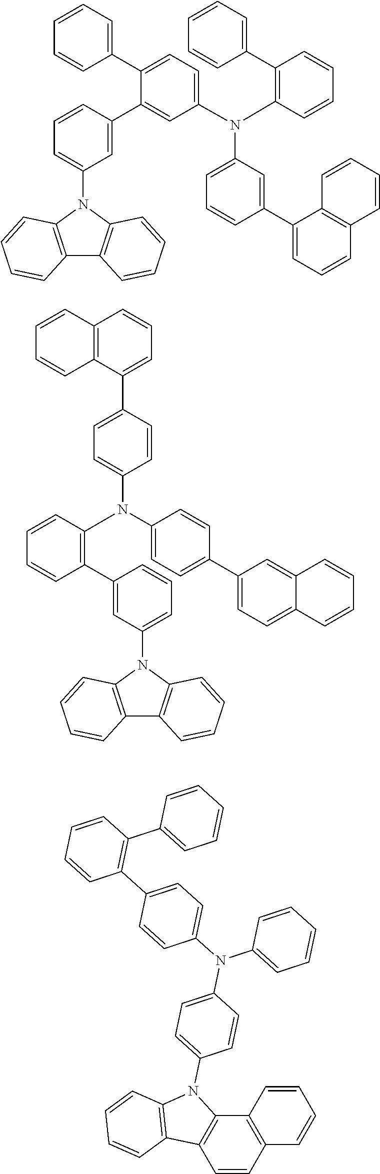 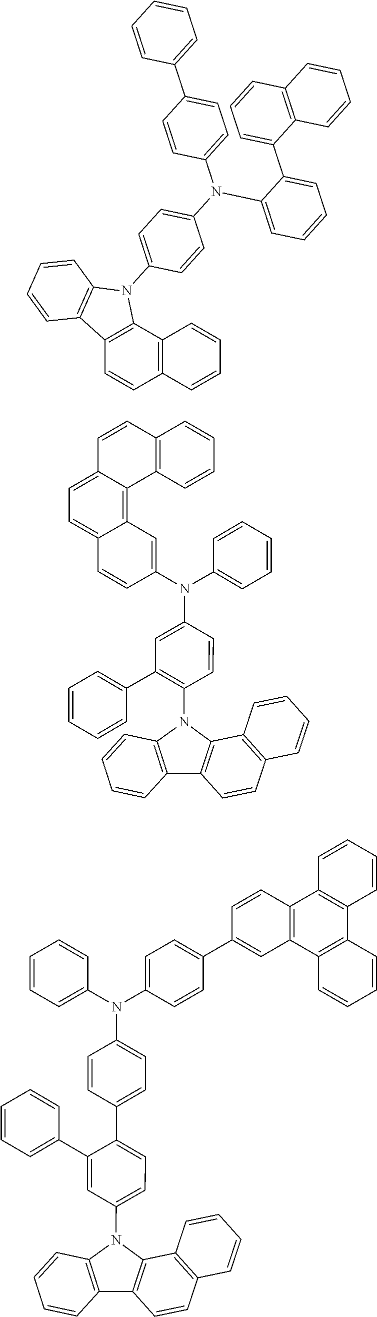 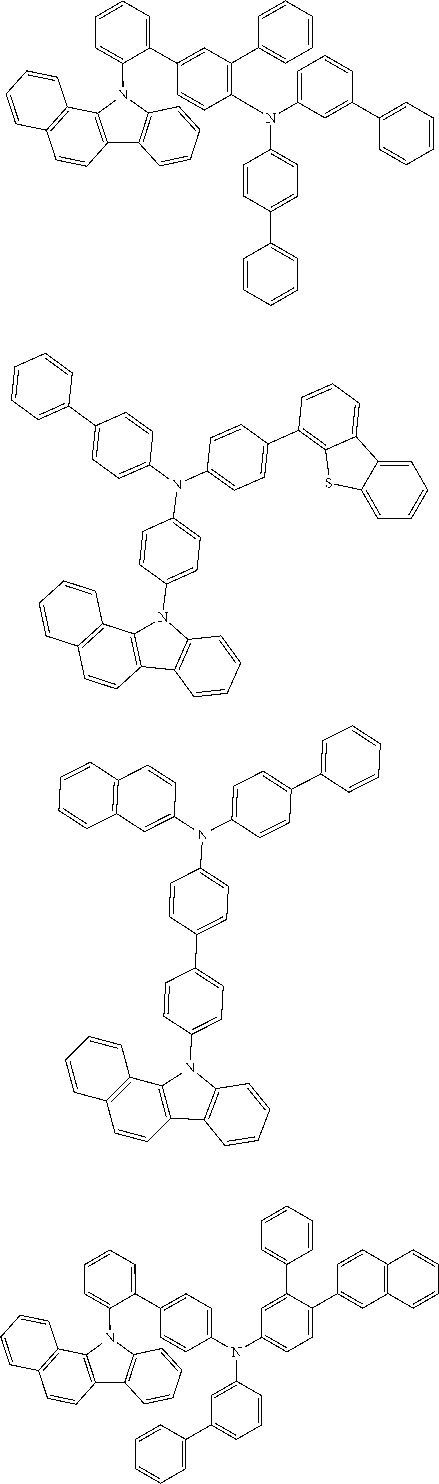 [SMILES: c1ccc(-c2ccc(N(c3cccc(-c4ccccc4)c3)c3ccc(-c4ccccc4-n4c5ccccc5c5ccc6ccccc6c54)cc3-c3ccccc3)cc2)cc1 ; c1ccc(-c2ccc(N(c3ccc(-c4cccc5c4sc4ccccc45)cc3)c3ccc(-n4c5ccccc5c5ccc6ccccc6c54)cc3)cc2)cc1 ; c1ccc(-c2ccc(N(c3ccc(-c4ccc(-n5c6ccccc6c6ccc7ccccc7c65)cc4)cc3)c3ccc4ccccc4c3)cc2)cc1 ; c1ccc(-c2cccc(N(c3ccc(-c4ccccc4-n4c5ccccc5c5ccc6ccccc6c54)cc3)c3ccc(-c4ccc5ccccc5c4)c(-c4ccccc4)c3)c2)cc1] 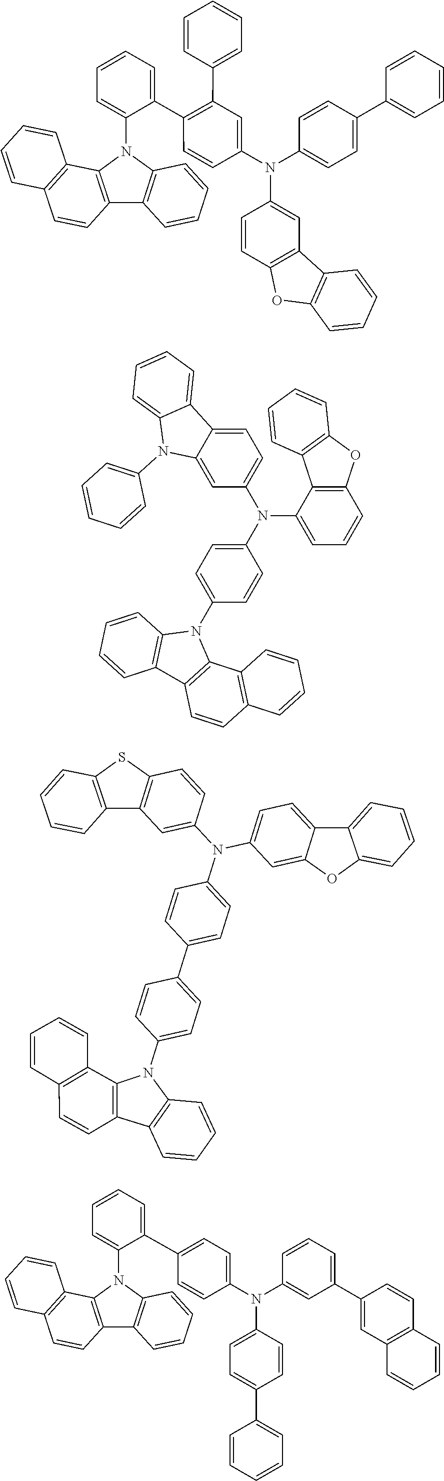 [SMILES: c1ccc(-c2ccc(N(c3ccc(-c4ccccc4-n4c5ccccc5c5ccc6ccccc6c54)c(-c4ccccc4)c3)c3ccc4oc5ccccc5c4c3)cc2)cc1 ; c1ccc(-n2c3ccccc3c3ccc(N(c4ccc(-n5c6ccccc6c6ccc7ccccc7c65)cc4)c4cccc5oc6ccccc6c45)cc32)cc1 ; c1ccc2c(c1)ccc1c3ccccc3n(-c3ccc(-c4ccc(N(c5ccc6c(c5)oc5ccccc56)c5ccc6sc7ccccc7c6c5)cc4)cc3)c21 ; c1ccc(-c2ccc(N(c3ccc(-c4ccccc4-n4c5ccccc5c5ccc6ccccc6c54)cc3)c3cccc(-c4ccc5ccccc5c4)c3)cc2)cc1] 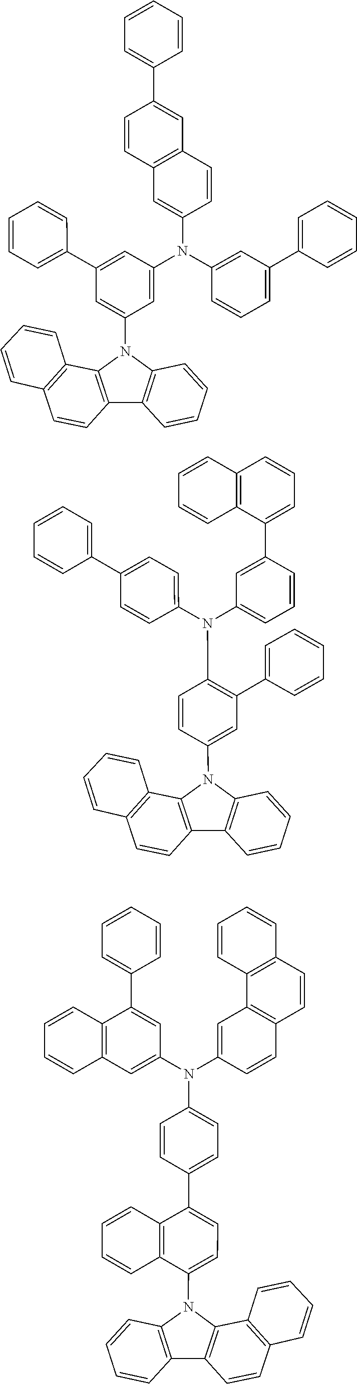 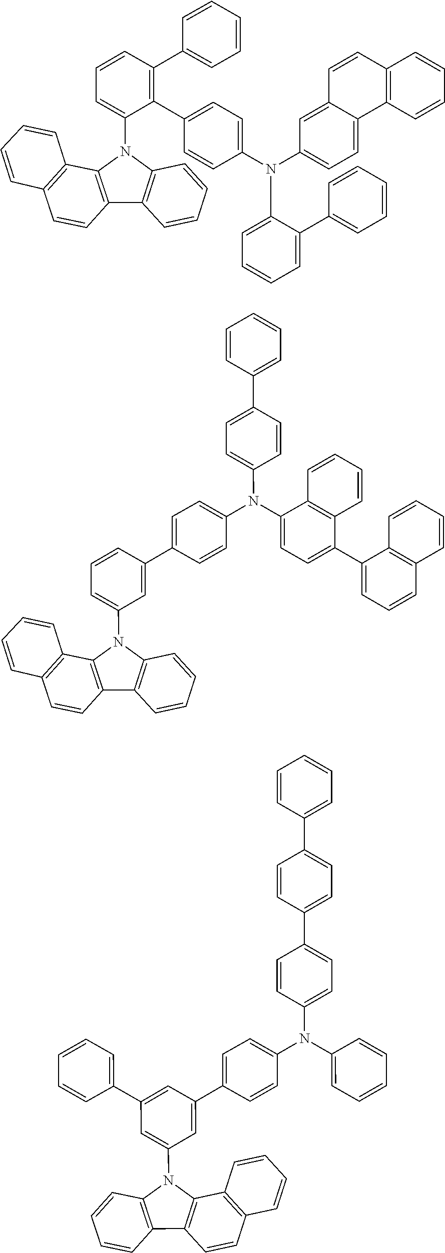 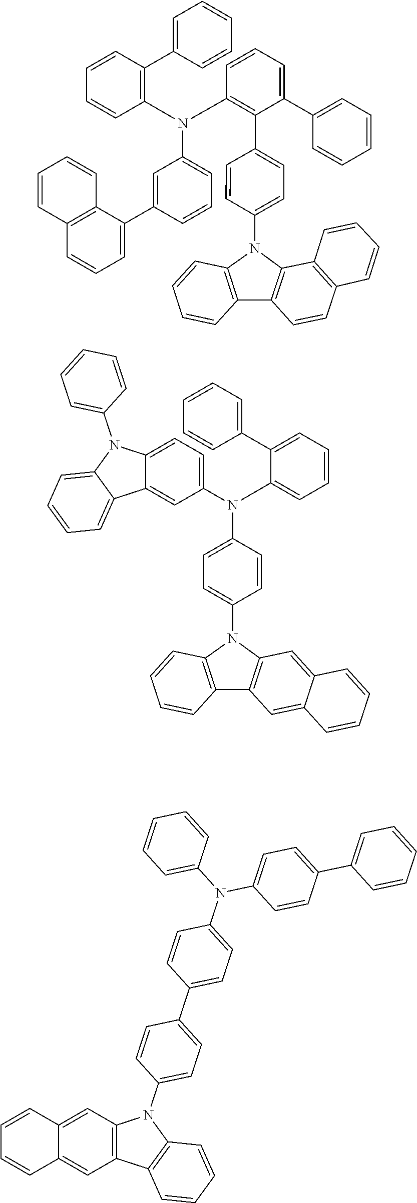 [SMILES: c1ccc(-c2ccccc2N(c2cccc(-c3cccc4ccccc34)c2)c2cccc(-c3ccccc3)c2-c2ccc(-n3c4ccccc4c4ccc5ccccc5c43)cc2)cc1 ; c1ccc(-c2ccccc2N(c2ccc(-n3c4ccccc4c4cc5ccccc5cc43)cc2)c2ccc3c(c2)c2ccccc2n3-c2ccccc2)cc1 ; c1ccc(-c2ccc(N(c3ccccc3)c3ccc(-c4ccc(-n5c6ccccc6c6cc7ccccc7cc65)cc4)cc3)cc2)cc1] 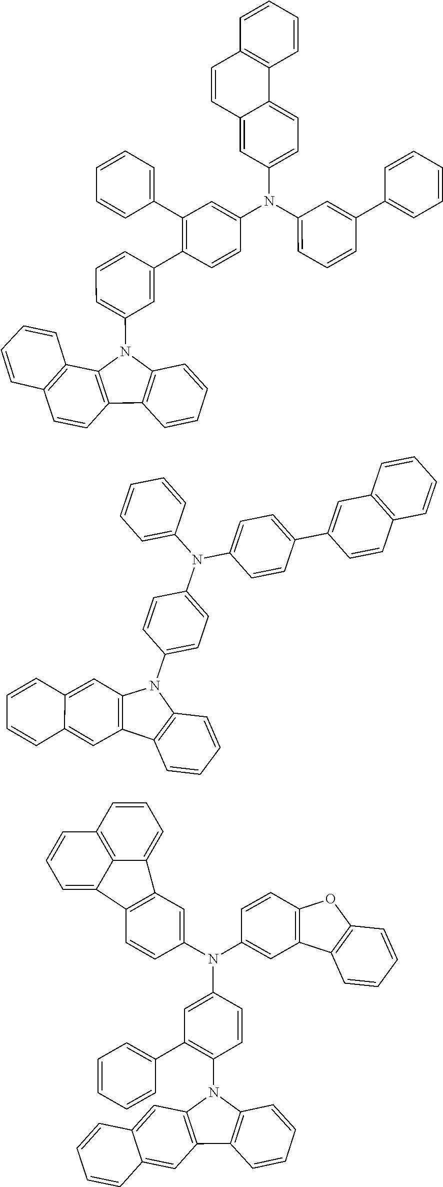 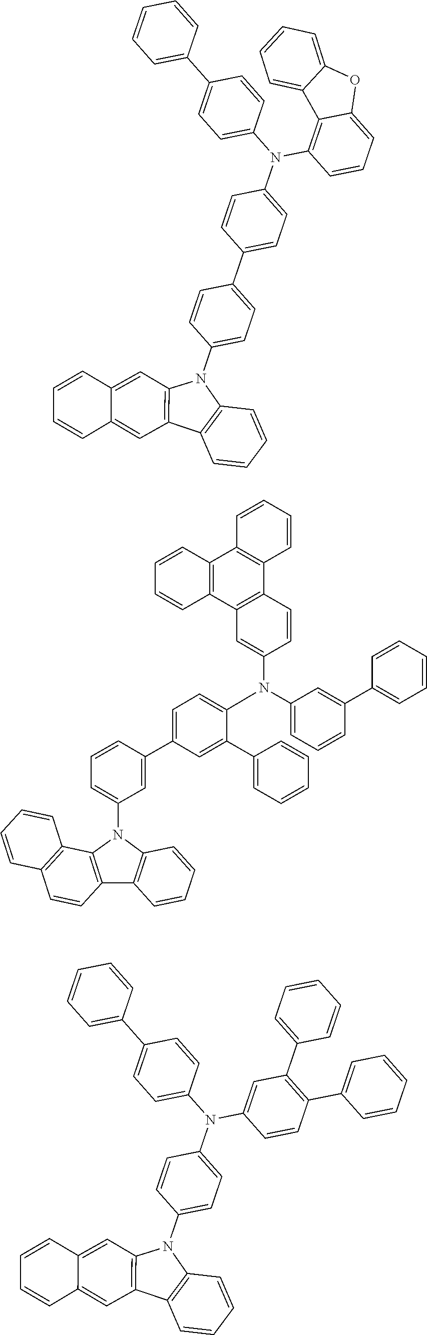 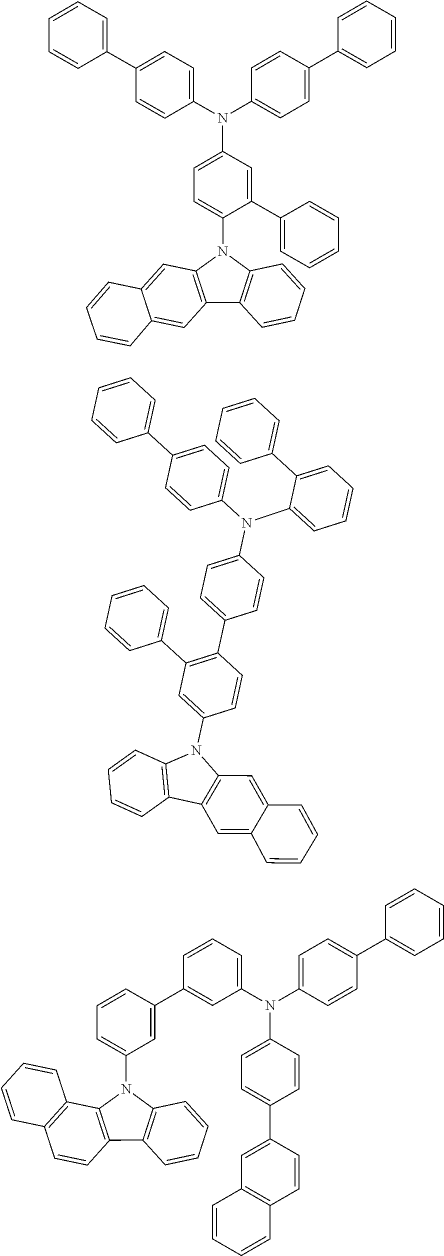 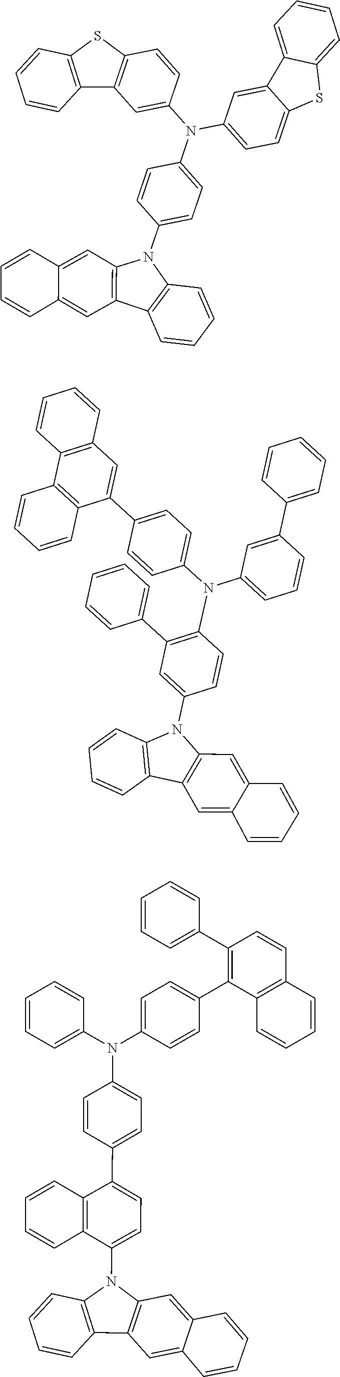 [SMILES: c1ccc2cc3c(cc2c1)c1ccccc1n3-c1ccc(N(c2ccc3sc4ccccc4c3c2)c2ccc3sc4ccccc4c3c2)cc1 ; c1ccc(-c2cccc(N(c3ccc(-c4cc5ccccc5c5ccccc45)cc3)c3ccc(-n4c5ccccc5c5cc6ccccc6cc54)cc3-c3ccccc3)c2)cc1 ; c1ccc(-c2ccc3ccccc3c2-c2ccc(N(c3ccccc3)c3ccc(-c4ccc(-n5c6ccccc6c6cc7ccccc7cc65)c5ccccc45)cc3)cc2)cc1] 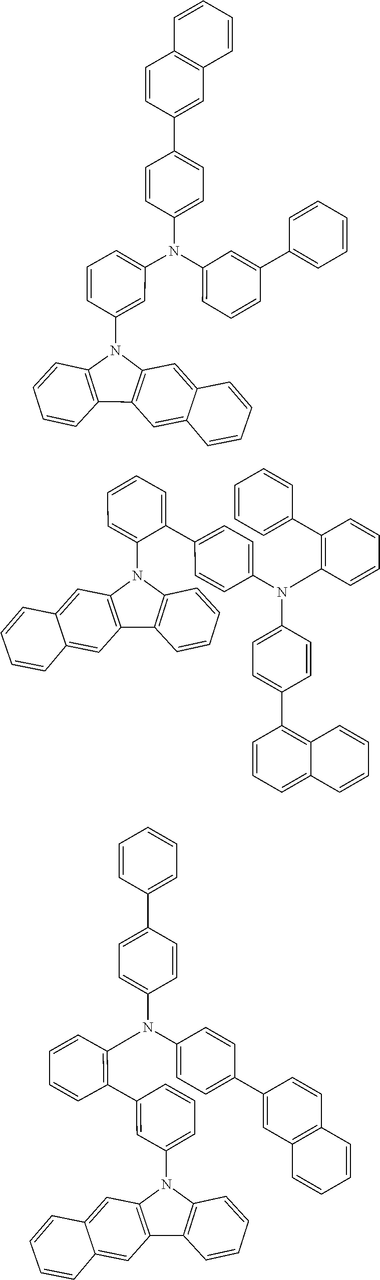 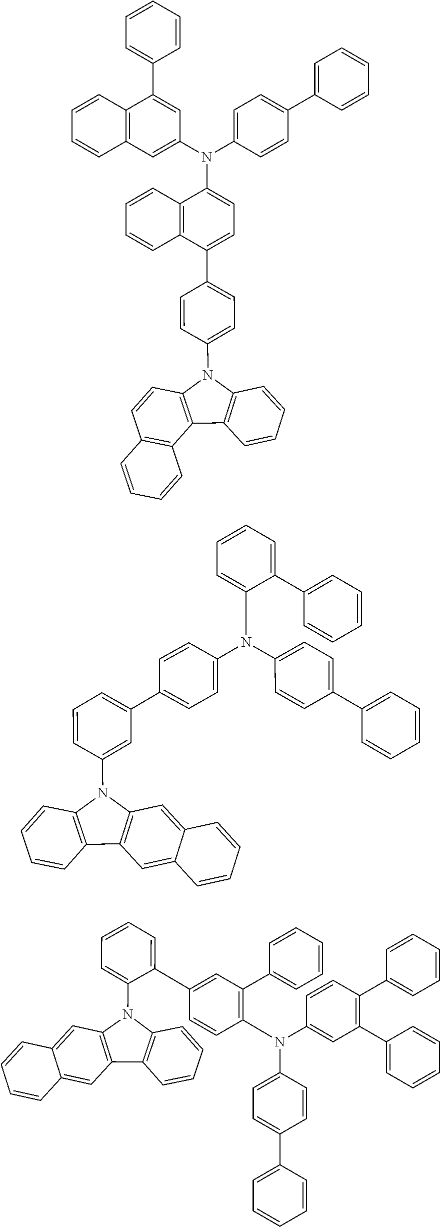 [SMILES: c1ccc(-c2ccc(N(c3cc(-c4ccccc4)c4ccccc4c3)c3ccc(-c4ccc(-n5c6ccccc6c6c7ccccc7ccc65)cc4)c4ccccc34)cc2)cc1 ; c1ccc(-c2ccc(N(c3ccc(-c4cccc(-n5c6ccccc6c6cc7ccccc7cc65)c4)cc3)c3ccccc3-c3ccccc3)cc2)cc1 ; c1ccc(-c2ccc(N(c3ccc(-c4ccccc4)c(-c4ccccc4)c3)c3ccc(-c4ccccc4-n4c5ccccc5c5cc6ccccc6cc54)cc3-c3ccccc3)cc2)cc1] 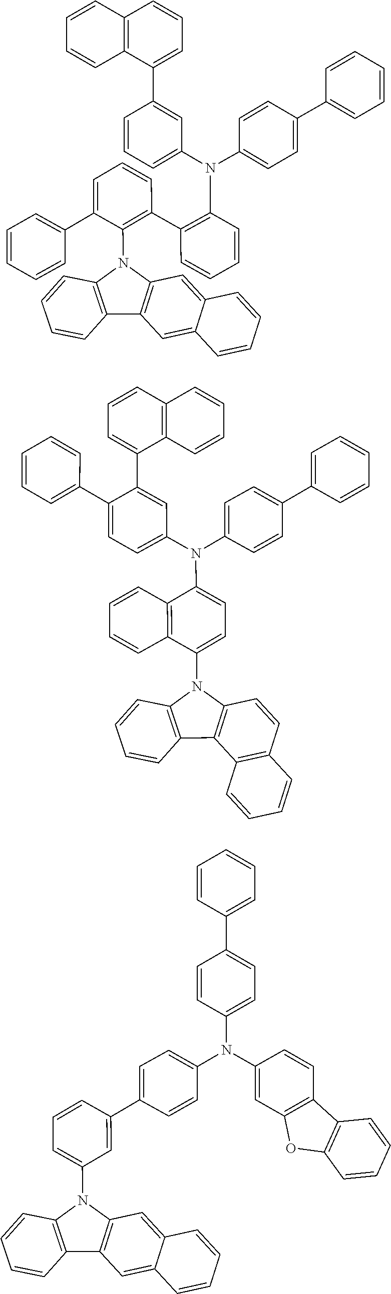 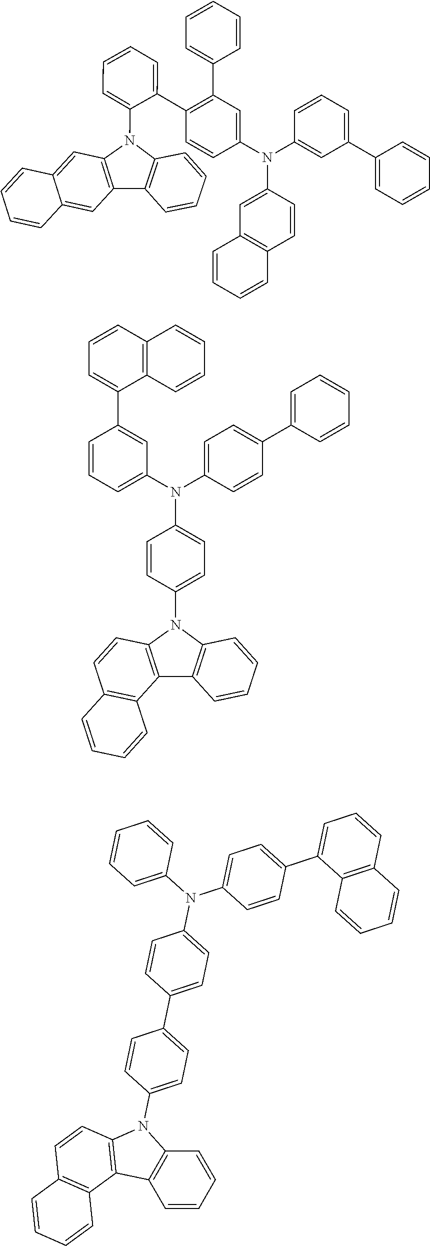 [SMILES: c1ccc(-c2cccc(N(c3ccc(-c4ccccc4-n4c5ccccc5c5cc6ccccc6cc54)c(-c4ccccc4)c3)c3ccc4ccccc4c3)c2)cc1 ; c1ccc(-c2ccc(N(c3ccc(-n4c5ccccc5c5c6ccccc6ccc54)cc3)c3cccc(-c4cccc5ccccc45)c3)cc2)cc1 ; c1ccc(N(c2ccc(-c3ccc(-n4c5ccccc5c5c6ccccc6ccc54)cc3)cc2)c2ccc(-c3cccc4ccccc34)cc2)cc1] 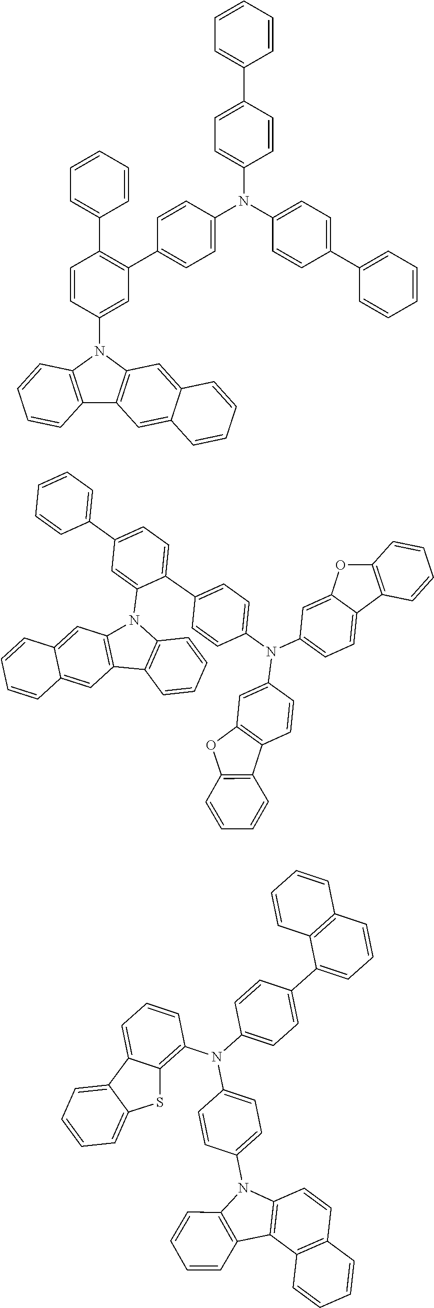 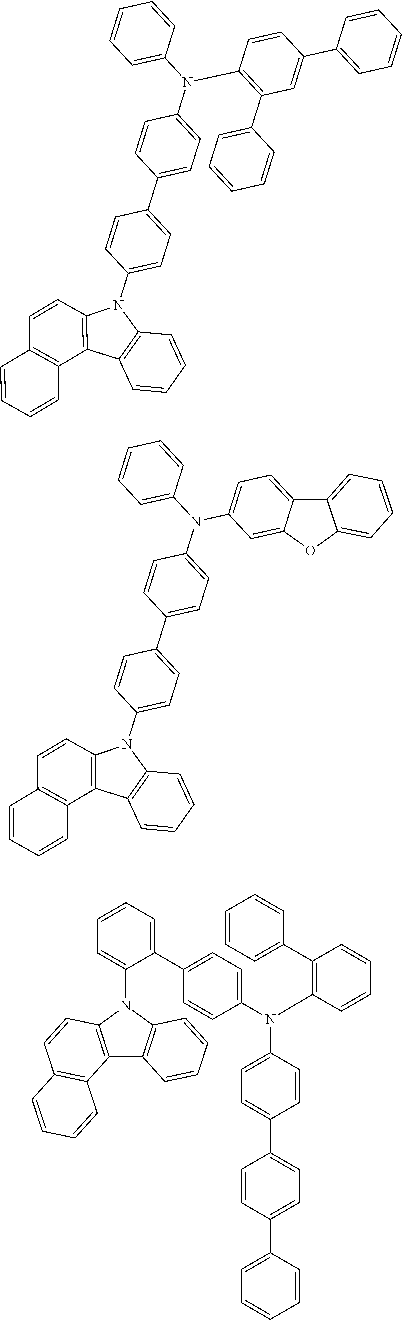 [SMILES: c1ccc(-c2ccc(N(c3ccccc3)c3ccc(-c4ccc(-n5c6ccccc6c6c7ccccc7ccc65)cc4)cc3)c(-c3ccccc3)c2)cc1 ; c1ccc(N(c2ccc(-c3ccc(-n4c5ccccc5c5c6ccccc6ccc54)cc3)cc2)c2ccc3c(c2)oc2ccccc23)cc1 ; c1ccc(-c2ccc(-c3ccc(N(c4ccc(-c5ccccc5-n5c6ccccc6c6c7ccccc7ccc65)cc4)c4ccccc4-c4ccccc4)cc3)cc2)cc1] 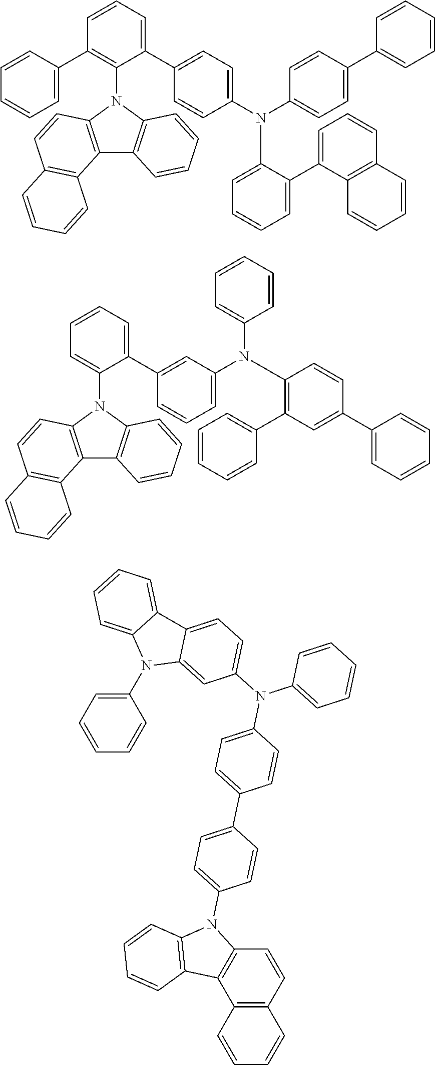 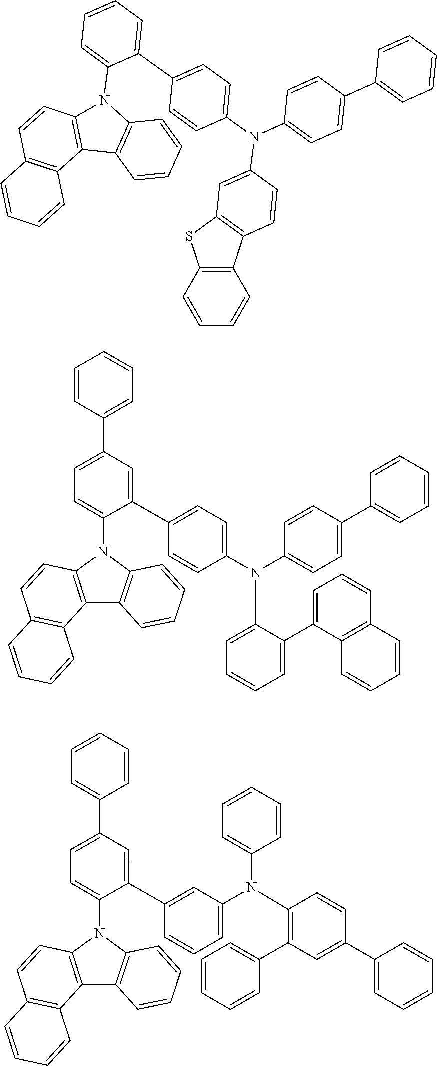 [SMILES: c1ccc(-c2ccc(N(c3ccc(-c4ccccc4-n4c5ccccc5c5c6ccccc6ccc54)cc3)c3ccc4c(c3)sc3ccccc34)cc2)cc1 ; c1ccc(-c2ccc(N(c3ccc(-c4cc(-c5ccccc5)ccc4-n4c5ccccc5c5c6ccccc6ccc54)cc3)c3ccccc3-c3cccc4ccccc34)cc2)cc1 ; c1ccc(-c2ccc(N(c3ccccc3)c3cccc(-c4cc(-c5ccccc5)ccc4-n4c5ccccc5c5c6ccccc6ccc54)c3)c(-c3ccccc3)c2)cc1] 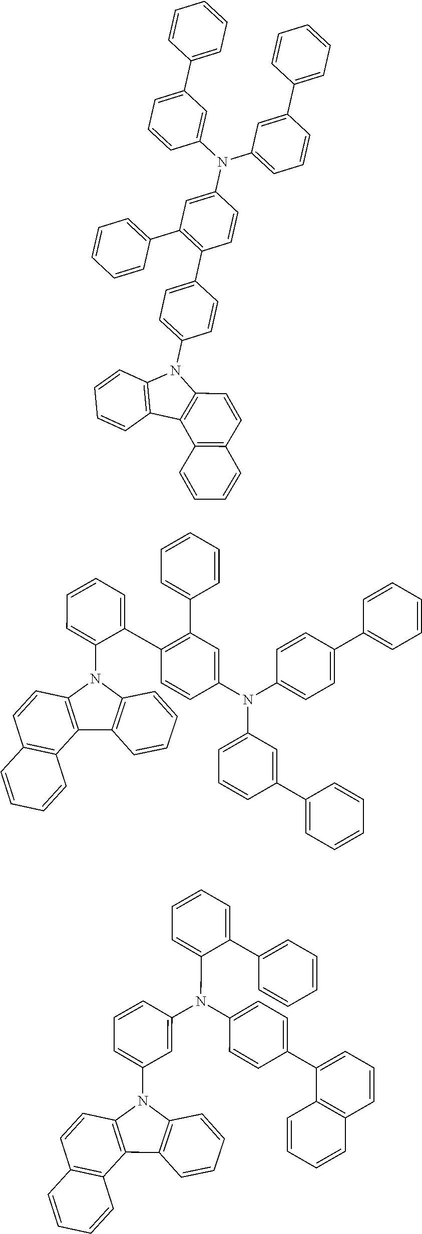 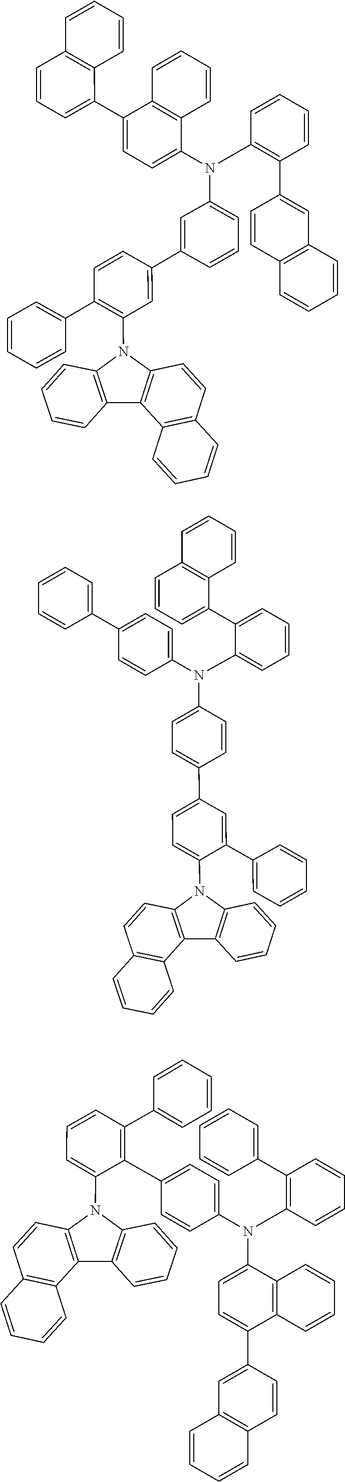 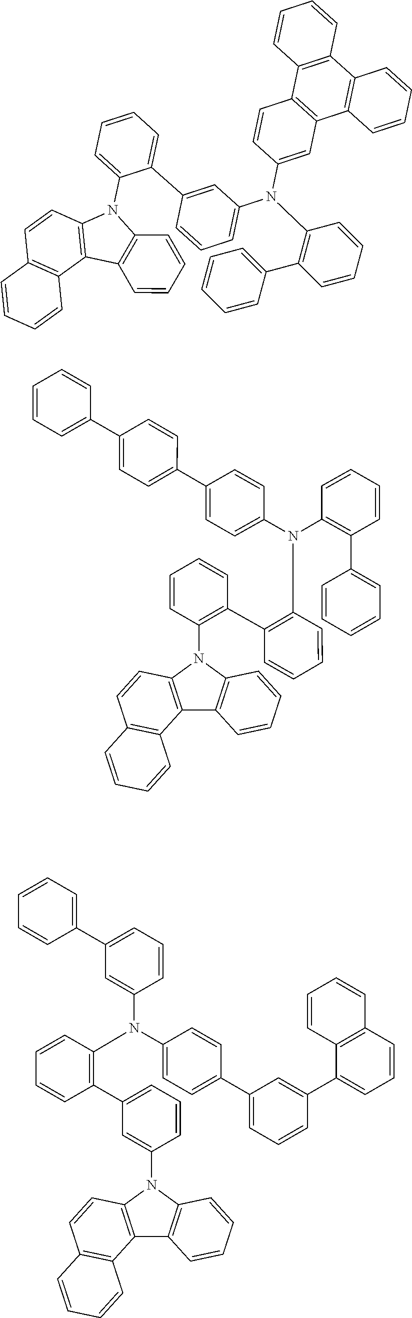 [SMILES: c1ccc(-c2ccccc2N(c2cccc(-c3ccccc3-n3c4ccccc4c4c5ccccc5ccc43)c2)c2ccc3c4ccccc4c4ccccc4c3c2)cc1 ; c1ccc(-c2ccc(-c3ccc(N(c4ccccc4-c4ccccc4)c4ccccc4-c4ccccc4-n4c5ccccc5c5c6ccccc6ccc54)cc3)cc2)cc1 ; c1ccc(-c2cccc(N(c3ccc(-c4cccc(-c5cccc6ccccc56)c4)cc3)c3ccccc3-c3cccc(-n4c5ccccc5c5c6ccccc6ccc54)c3)c2)cc1] 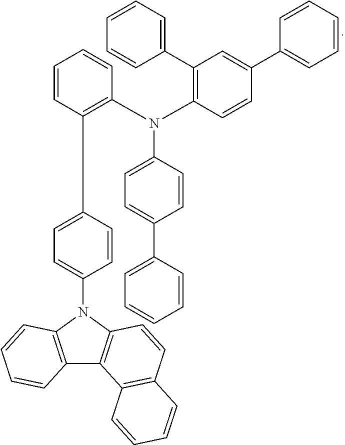 [SMILES: c1ccc(-c2ccc(N(c3ccccc3-c3ccc(-n4c5ccccc5c5c6ccccc6ccc54)cc3)c3ccc(-c4ccccc4)cc3-c3ccccc3)cc2)cc1]
